(19) United States
(12) Patent Application Publication
(10) Pub. No.: US 2024/0050428 A1
(43) Pub. Date: Feb. 15, 2024

(54) COMPOUNDS AND METHODS OF TREATING CANCERS

(71) Applicant: Cullgen (Shanghai), Inc., Shanghai (CN)

(72) Inventors: Jing LIU, Oradell, NJ (US); Michael Bruno PLEWE, San Diego, CA (US); Jialiang WANG, Nashville, TN (US); Xiaoran HAN, Shanghai (CN); Liqun CHEN, Shanghai (CN); Ting YANG, Shanghai (CN); Chengwei ZHANG, Shanghai (CN)

(21) Appl. No.: 18/248,035

(22) PCT Filed: Oct. 7, 2021
(86) PCT No.: PCT/CN2021/122530

§ 371 Date: Apr. 5, 2023

(30) Foreign Application Priority Data
Oct. 7, 2020 (WO) PCT/CN2020/119834

(51) Int. Cl. A61K 31/498; A61K 31/4545; A61K 31/454; A61K 31/5025; A61K 31/497; A61K 45/06; A61P 35/00

(52) U.S. Cl. CPC A61K 31/498; A61K 31/4545; A61K 31/454; A61K 31/5025; A61K 31/497; A61K 45/06; A61P 35/00

(57) ABSTRACT

This disclosure relates to GSPT1 degrader compounds (e.g. small molecule compounds), compositions comprising one or more of the compounds, and to methods of use the compounds for the treatment of certain disease in a subject in need thereof. The disclosure also relates to methods for identifying such compounds.

### CROSS-REFERENCE

This application claims the benefit of PCT Application No. PCT/CN2020/119834, filed Oct. 7, 2020, which is incorporated herein by reference in its entirety.

### BACKGROUND

There is a need in the art for compounds, compositions, and methods of use of the compounds for the treatment of diseases in a subject in need thereof.

### SUMMARY

This disclosure relates to compounds (e.g. small molecule compounds), compositions comprising one or more of the compounds, and to methods of use of the compounds for the treatment of certain diseases in a subject in need thereof. The disclosure also relates to methods for identifying or making such compounds.

In some embodiments, are methods of treatment, comprising: administering to a subject in need thereof, a first compound comprising a GSPT1 degrader and a second compound comprising an FLT3 pathway inhibitor, a RAS-RAF-MEK-ERK pathway inhibitor, or a PI3K-AKT-mTOR pathway inhibitor or activator.

In some embodiments, provided herein are compounds having the structure of FORMULA 1, or a pharmaceutically acceptable salt or solvate thereof:

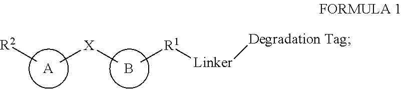

wherein R¹ is a divalent group that is connected to a linker moiety, and is absent, or selected from R′—R″, R′OR″, R′SR″, R′N(R³)R″, R′OC(O)R″, R′OC(O)OR″, R′OCON(R³)R″, R′C(O)R″, R′C(O)OR″, R′CON(R³)R″, R′S(O)R″, R′S(O)₂R″, R′SO₂N(R³)R″, R′NR⁴C(O)OR″, R′NR⁴C(O)R″, R′NR⁴C(O)N(R³)R″, R′NR⁴S(O)R″, R′NR⁴S(O)₂R″, and R′NR⁴S(O)₂NR³R″, wherein R′ and R″ are each divalent groups that are independently absent or selected from an optionally substituted C₁-C₈ alkylene, optionally substituted C₂-C₈ alkenylene, optionally substituted C₂-C₈ alkynylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl; R³ and R⁴ are independently selected from an optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted 3-10 membered carbocyclyl, optionally substituted 3-10 membered heterocyclyl, optionally substituted aryl, and optionally substituted heteroaryl; or R′ and R″, R³ and R⁴, R′ and R³, R′ and R⁴, R″ and R³, or R″ and R⁴, together with the atom(s) to which they are attached, form a 3-20 membered carbocyclyl or 3-20 membered heterocyclyl ring; R² is selected from hydrogen, halogen, oxo, CN, NO₂, OR⁵, SR⁵, N(R⁵)R⁶, OCOR⁵, OCO₂R⁵, OCON(R⁵)R⁶, COR⁵, CO₂R⁵, CON(R⁵)R⁶, SOR⁵, SO₂R⁵, SO₂N(R⁵)R⁶, NR⁷CO₂R⁵, NR⁷COR⁵, NR⁷C(O)N(R⁵)R⁶, NR⁷SOR⁵, NR⁷SO₂R⁵, NR⁷SO₂N(R⁵)R⁶, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered heterocyclylC₁-C₈alkyl, optionally substituted 3-10 membered carbocyclylC₁-C₈alkyl, optionally substituted 3-10 membered carbocyclyl, optionally substituted 3-10 membered heterocyclyl, optionally substituted aryl, and optionally substituted heteroaryl, wherein each R⁵, R⁶, and R⁷ is independently selected from hydrogen, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, and optionally substituted heteroaryl; or R and R⁶ or R and R together with the atom to which they are attached form a 3-20 membered heterocyclyl, or 5-6 membered heteroaryl; X is absent or is selected from a divalent group comprising CR⁸R⁹, CO, CO₂, CONR⁸, NR⁸, NR⁸CO, NR⁸CO₂, NR⁸C(O)NR⁹, NR⁸SO, NR⁸SO₂, NR⁸SO₂NR⁹, O, OC(O), OCO₂, OCONR⁸, S, SO, SO₂, and SO₂NR⁸, optionally substituted C₁-C₈ alkylene, optionally substituted C₂-C₈ alkenylene, optionally substituted C₂-C₈ alkynylene, optionally substituted 3-10 membered carbocyclyl, optionally substituted 3-10 membered heterocyclyl, optionally substituted aryl, and optionally substituted heteroaryl, wherein each R⁸ and R⁹ is independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈ alkoxy, optionally substituted C₁-C₈ alkylamino, optionally substituted 3-10 membered carbocyclyl, optionally substituted 3-10 membered heterocyclyl, optionally substituted aryl, and optionally substituted heteroaryl; or R⁸ and R⁹ together with the atom(s) to which they are attached form a 3-20 membered carbocyclyl or 3-20 membered heterocyclyl ring; and ring A

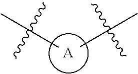

and ring B

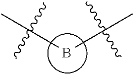

are each independently absent or selected from an optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, optionally substituted bicyclic fused aryl, optionally substituted tricyclic fused aryl, and optionally substituted heteroaryl, optionally substituted bicyclic fused heteroaryl, and optionally substituted tricyclic fused heteroaryl.

In some embodiments, provided herein are compounds having the structure of FORMULA 1, or a pharmaceutically acceptable salt or solvate thereof:

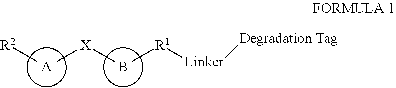

wherein, ring A is absent, or an optionally substituted 3-10 membered carbocyclyl, optionally substituted 3-10 membered heterocyclyl, optionally substituted aryl, optionally substituted bicyclic aryl, optionally substituted heteroaryl, or optionally substituted bicyclic heteroaryl; ring B is absent, or an optionally substituted 3-10 membered carbocyclyl, optionally substituted 3-10 membered heterocyclyl, optionally substituted aryl, or optionally substituted heteroaryl; R¹ is absent; R² is hydrogen, halogen, CN, NO₂, OR⁵, SR⁵, N(R⁵)R⁶, C(O)R⁵, C(O)OR⁵, C(O)N(R⁵)R⁶, SO₂R⁵, SO₂N(R⁵)R⁶, NR⁷C(O)OR⁵, NR⁷C(O)R⁵, NR⁷C(O)N(R⁵)R⁶, NR⁷SOR⁵, NR⁷SO₂R⁵, NR⁷SO₂N(R⁵)R⁶ optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ alkoxy, or optionally substituted C₁-C₈ alkylamino, optionally substituted 3-10 membered carbocyclyl, or optionally substituted 3-10 membered heterocyclyl; X is absent, —O—, or NR⁸; each R⁵, R⁶ and R⁷ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or R⁵ and R⁶ together with the atom(s) to which they are attached optionally form a 3-20 membered heterocyclyl or 5-6 membered heteroaryl ring; R⁸ is hydrogen or C₁-C₈ alkyl; and Linker is a linking moiety connecting the degradation tag to ring B.

In some embodiments, provided herein are pharmaceutical compositions comprising a compound described herein, and a pharmaceutically acceptable excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A includes an image of a western blot for GSPT1 after MV4;11 cells were treated with GS-001 or GS-002.

FIG. 1B includes an image of a western blot for GSPT1 after MV4;11 cells were treated with GS-004, GS-005 or GS-006.

FIG. 1C includes an image of a western blot for GSPT1 after MV4;11 cells were treated with GS-007, GS-008, GS-009, GS-010, GS-011, GS-012 or GS-013.

FIG. 1D includes an image of a western blot for GSPT1 after MV4;11 cells were treated with GS-017, GS-018 or GS-019.

FIG. 1E includes an image of a western blot for GSPT1 after MV4;11 cells were treated with GS-020, GS-021, GS-022, GS-060, GS-062, GS-063 or GS-064.

FIG. 2A is a graph of cell viability in MOML-13 cells treated with varying doses of GS-002, with or without gilteritinib at 20 nM.

FIG. 2B is a graph of cell viability in MOML-13 cells treated with varying doses of GS-005, with or without gilteritinib at 20 nM.

FIG. 2C is a graph of cell viability in MOML-13 cells treated with varying doses of GS-006, with or without gilteritinib at 20 nM.

### DETAILED DESCRIPTION

Termination of translation is a GTP-dependent process that is regulated by two key proteins eRF1 and eRF3. The translation termination factor eRF3a (also known as eukaryotic peptide chain release factor GTP-binding subunit ERF3A, or “GSPT1”) is a GTPase that interacts with eRF1 to promote stop codon recognition and release of nascent peptide from ribosome (Chauvin, Salhi et al. 2005). GSPT1 activates eRF1 in a GTP-dependent manner and its GTPase activity requires complexing with eRF1 and ribosomes (Frolova, Le Goff et al. 1996). The GTP-bound GSPT1 and eRF1 together with ribosomes form the functional translation termination complexes (Zhouravleva, Frolova et al. 1995). Through regulation of translation, GSPT1 has diverse and important roles in cell physiology. Increased expression of GSPT1 has been reported in human malignancies, including lung cancer and gastric cancer (Malta-Vacas, Aires et al. 2005, Tian, Tian et al. 2018, Sun, Zhang et al. 2019, Zhang, Zou et al. 2019). Hence, GSPT1 is thought to be a novel cancer target through which one may compromise active translation that contributes to malignant phenotypes of cancer cells. Recently, Matyskiela and colleagues have reported that a phthalimide-derived molecule CC-885 led to cereblon-dependent degradation of GSPT1 and other targets, such as IKZF1 and IKZF3 (Matyskiela, Lu et al. 2016). Ishoey et al. also reported that GSPT1 was degraded by a subset of heterobiofunctional compounds derived from phthalimide (Ishoey, Chorn et al. 2018). CC-885 induced significant toxicity in the vast majority of tested cell lines, presumably due to degrading GSTP1 and many other proteins (Matyskiela, Lu et al. 2016). Therefore, despite the broad and potent anti-cancer activity, CC-885 exhibits unacceptable toxicity that prevents further development (Hansen, Correa et al. 2020).

Disclosed herein, in some embodiments, are compounds. In some embodiments, the compound comprises a chemical structure or formula disclosed herein. The compound may be or include a GSPT1 degrader. GSPT1 degraders may be characterized by the ability to degrade or reduce cellular protein levels of GSPT1. Some embodiments relate to a composition that includes the compound. Some embodiments relate to methods of making the compound. Some embodiments relate to methods of using the compound or a pharmaceutical composition of the compound. For example, the compound may be used to treat a disorder or a disease. In some cases, the compound is used to treat cancer.

This disclosure includes all stereoisomers, geometric isomers, tautomers and isotopes of the structures depicted and compounds named herein. This disclosure also includes compounds described herein, regardless of how they are prepared, e.g., synthetically, through biological process (e.g., metabolism or enzyme conversion), or a combination thereof.

This disclosure includes pharmaceutically acceptable salts of the structures depicted and compounds named herein.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms. In some embodiments, the compound includes at least one fluorine atom. In some embodiments, the compound includes two or more fluorine atoms. In some embodiments, the compound includes 1-2, 1-3, 1-4, 1-5, or 1-6 fluorine atoms. In some embodiments, all of the hydrogen atoms in a compound can be replaced or substituted by fluorine atoms.

### I. Compounds

Disclosed herein, in some embodiments, are compounds. In some embodiments, the compound comprises a Degradation Tag disclosed herein. In some embodiments, the compound comprises a cereblon-binding moiety. In some embodiments, the Degradation Tag comprises the cereblon-binding moiety. In some embodiments, the compound comprises a GSPT1 degrader. For example, the compound may result in GSPT1 degradation. The compound may degrade GSPT1 as a result of cereblon modulation by the Degradation Tag. The compound may bind to or modulate GSPT1 or cereblon. In some embodiments, the compound comprises a heterobifunctional compound. In some embodiments, the compound comprises a molecular glue. In some embodiments, the compound may be used as a molecular glue. In some embodiments, the compound comprises a linker. In some embodiments, the compound comprises a truncated Janus kinase (JAK)-binding moiety.

In some embodiments, a compound disclosed herein comprises FORMULA 1, or a pharmaceutically acceptable salt or solvate thereof:

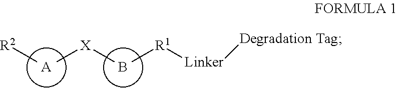

wherein R¹ is a divalent group that is connected to a linker moiety, and is absent, or selected from R′—R″, R′OR″, R′SR″, R′N(R³)R″, R′OC(O)R″, R′OC(O)OR″, R′OCON(R³)R″, R′C(O)R″, R′C(O)OR″, R′CON(R³)R″, R′S(O)R″, R′S(O)₂R″, R′SO₂N(R³)R″, R′NR⁴C(O)OR″, R′NR⁴C(O)R″, R′NR⁴C(O)N(R³)R″, R′NR⁴S(O)R″, R′NR⁴S(O)₂R″, and R′NR⁴S(O)₂NR³R″, wherein R′ and R″ are each divalent groups that are independently absent or selected from an optionally substituted C₁-C₈ alkylene, optionally substituted C₂-C₈ alkenylene, optionally substituted C₂-C₈ alkynylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl; R³ and R⁴ are independently selected from an optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted 3-10 membered carbocyclyl, optionally substituted 3-10 membered heterocyclyl, optionally substituted aryl, and optionally substituted heteroaryl; or R′ and R″, R³ and R⁴, R′ and R³, R¹ and R⁴, R″ and R³, or R″ and R⁴, together with the atom(s) to which they are attached, form a 3-20 membered carbocyclyl or 3-20 membered heterocyclyl ring; R² is selected from hydrogen, halogen, oxo, CN, NO₂, OR⁵, SR⁵, N(R⁵)R⁶, OCOR⁵, OCO₂R⁵, OCON(R⁵)R⁶, COR⁵, CO₂R⁵, CON(R⁵)R⁶, SOR⁵, SO₂R⁵, SO₂N(R⁵)R⁶, NR⁷CO₂R⁵, NR⁷COR⁵, NR⁷C(O)N(R⁵)R⁶, NR⁷SOR⁵, NR⁷SO₂R⁵, NR⁷SO₂N(R⁵)R⁶, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered heterocyclylC₁-C₈alkyl, optionally substituted 3-10 membered carbocyclylC₁-C₈alkyl, optionally substituted 3-10 membered carbocyclyl, optionally substituted 3-10 membered heterocyclyl, optionally substituted aryl, and optionally substituted heteroaryl, wherein each R⁵, R⁶, and R⁷ is independently selected from hydrogen, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, and optionally substituted heteroaryl; or R⁵ and R⁶ or R⁵ and R⁷ together with the atom to which they are attached form a 3-20 membered heterocyclyl, or 5-6 membered heteroaryl; X is absent or is selected from a divalent group comprising CR⁸R⁹, CO, CO₂, CONR⁸, NR⁸, NR⁸CO, NR⁸CO₂, NR⁸C(O)NR⁹, NR⁸SO, NR⁸SO₂, NR⁸SO₂NR⁹, O, OC(O), OCO₂, OCONR⁸, S, SO, SO₂, and SO₂NR⁸, optionally substituted C₁-C₈ alkylene, optionally substituted C₂-C₈ alkenylene, optionally substituted C₂-C₈ alkynylene, optionally substituted 3-10 membered carbocyclyl, optionally substituted 3-10 membered heterocyclyl, optionally substituted aryl, and optionally substituted heteroaryl, wherein each R⁸ and R⁹ is independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈ alkoxy, optionally substituted C₁-C₈ alkylamino, optionally substituted 3-10 membered carbocyclyl, optionally substituted 3-10 membered heterocyclyl, optionally substituted aryl, and optionally substituted heteroaryl; or R⁸ and R⁹ together with the atom(s) to which they are attached form a 3-20 membered carbocyclyl or 3-20 membered heterocyclyl ring; and ring A

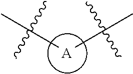

and ring B

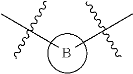

are each independently absent or selected from an optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, optionally substituted bicyclic fused aryl, optionally substituted tricyclic fused aryl, and optionally substituted heteroaryl, optionally substituted bicyclic fused heteroaryl, and optionally substituted tricyclic fused heteroaryl.

Some embodiments include a Degradation Tag. In some embodiments, the Degradation Tag includes a cereblon-binding moiety. The Degradation Tag may bind to cereblon. Without wishing to be bound by any particular theory, it is contemplated herein that, in some embodiments, attaching pomalidomide to either portion of the molecule can recruit the cereblon E3 ligase. The Degradation Tag may include pomalidomide or a functional fragment thereof. In some embodiments, the Degradation Tag is optional.

In some embodiments, the degradation tag is a moiety comprising structural FORMULA 5, and wherein the degradation tag is connected to the linker moiety through Z_{E};

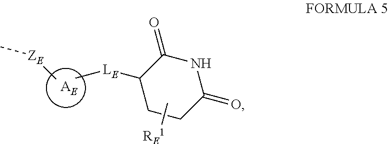

Wherein Z_{E} is a divalent group —(R_{E}^{Z})_{nE}—; wherein n_{E} is 0, 1, 2, 3, 4, 5 or 6; R_{E}^{Z}, at each occurrence, is independently R_{E}^{r}, or R_{E}^{W}; wherein R_{E}^{W}, at each occurrence, is a bond or selected from the group consisting of —C(O)—, —CR_{E}⁵R_{E}⁶—, —NR_{E}⁵—, —O—, optionally substituted C₁-C₁₀ alkylene, optionally substituted C₁-C₁₀ alkenylene, optionally substituted C₁-C₁₀ alkynylene; and R_{E}^{r}, at each occurrence, is a bond, or selected from the group consisting of optionally substituted 3-10 membered carbocyclyl (such as 3-8 membered carbocyclyl), optionally substituted 3-10 membered heterocyclyl, optionally substituted C₃-C₁₃ fused carbocyclyl, optionally substituted C₃-C₁₃ fused heterocyclyl, optionally substituted C₃-C₁₃ bridged carbocyclyl, optionally substituted C₃-C₁₃ bridged heterocyclyl, optionally substituted C₃-C₁₃ spiro carbocyclyl, optionally substituted C₃-C₁₃ spiro heterocyclyl, optionally substituted aryl, and optionally substituted heteroaryl; with the proviso that —R_{E}^{z}—R_{E}^{z}— is not —O—O—; R_{E}⁵ and R_{E}⁶ at each occurrence are independently selected from the group comprising hydrogen, halogen, oxo, hydroxy, amino, cyano, nitro, optionally substituted C₁-C₆ alkyl, optionally substituted C₁-C₈ heteroalkyl, optionally substituted C₁-C₈ haloalkyl, optionally substituted 3 to 8 membered carbocyclyl, and optionally substituted 3 to 8 membered heterocyclyl; or R_{E}⁵ and R_{E}⁶ together with the atom(s) to which they are attached form an optionally substituted 3-8 membered carbocyclyl or heterocyclyl ring; R_{E}¹ is selected from the group consisting of hydrogen, halogen, cyano, nitro, optionally substituted C₁-C₆ alkyl, optionally substituted C₁-C₈ heteroalkyl, optionally substituted C₁-C₈ haloalkyl, optionally substituted 3-8 membered carbocyclyl, and optionally substituted 3-8 membered heterocyclyl; L_{E} is a divalent group selected from the group consisting of absent, -L_{E}¹-, and -L_{E}¹-L_{E}²-; wherein L_{E}¹ and L_{E}² are independently selected from the group consisting of —CO—, —O—, —CR_{E}¹⁰R_{E}¹¹— and —NR_{E}¹⁰—, with the proviso that -L_{E}¹-L_{E}² is not —O—O—; wherein R_{E}¹⁰ and R_{E}¹¹ are independently selected from the group consisting of hydrogen, halogen, cyano, nitro, hydroxy, amino, optionally substituted C₁-C₆ alkyl, optionally substituted C₁-C₈ heteroalkyl, optionally substituted C₁-C₈ haloalkyl, optionally substituted C₁-C₆ alkoxy, and optionally substituted C₁-C₆ alkylamino; and Ring A_{E}

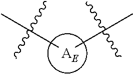

is a divalent group selected from the group consisting of FORMULA A_{E}1, A_{E}2, A_{E}3, A_{E}4 and A_{E}5:

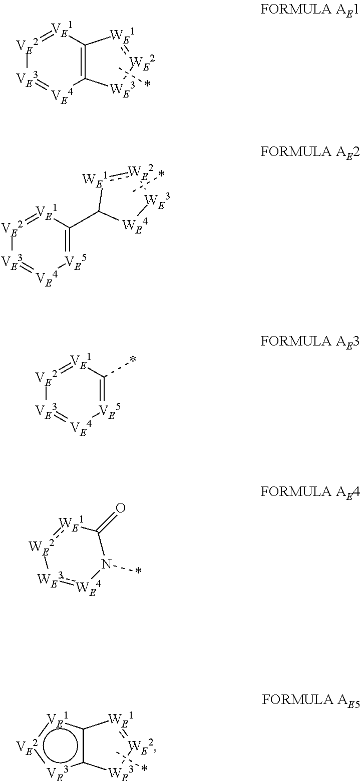

wherein * denotes the attachment to L_{E} or to Ring A_{E} when L_{E} is absent, and Z_{E} is attached to any possible position on the Ring A_{E}, [symbol] denotes a single bond or a double bond; V_{E}¹, V_{E}², V_{E}³, V_{E}4 and V_{E}⁵, at each occurrence, are independently selected from the group consisting of a bond, C, CR_{E}², S, N, and NR_{E}²; or V_{E}¹ and V_{E}², V_{E}² and V_{E}³, V_{E}³ and V_{E}⁴, or V_{E}⁴ and V_{E}⁵ are combined together with atom(s) to which they are attached to optionally form a 6 membered aryl or 5, 6 or 7 membered heteroaryl ring; R_{E}², at each occurrence, is independently selected from the group consisting of absent, hydrogen, halogen, cyano, nitro, hydroxy, amino, optionally substituted C₁-C₆ alkyl, optionally substituted C₁-C₆ alkenyl, optionally substituted C₁-C₆ alkynyl, optionally substituted C₁-C₈ heteroalkyl, optionally substituted C₁-C₈ haloalkyl, optionally substituted C₁-C₆ alkoxy, optionally substituted C₁-C₆ alkylamino, optionally substituted 3-8 membered carbocyclyl, and optionally substituted 3-8 membered heterocyclyl; or R_{E}² and another R_{E}² together with the atom(s) to which they are connected form optionally substituted 3-8 membered carbocyclyl, optionally substituted 3-8 membered heterocyclyl, optionally substituted aryl, and optionally substituted heteroaryl; W_{E}¹, W_{E}², W_{E}³ and W_{E}⁴ are each independently selected from the group consisting of —N═, —C≡, —CR_{E}³═, —CO—, —O—, —CR_{E}³R_{E}⁴—, —NR_{E}³—, —CR_{E}³═CR_{E}⁴—, —N═CR_{E}³—, and —N═N—; or W_{E}¹ and W_{E}², W_{E}² and W_{E}³, or W_{E}³ and W_{E}⁴ are combined together with the atom(s) to which they are attached to optionally form a 6 membered aryl or 5, 6 or 7 membered heteroaryl ring; and R_{E}³ and R_{E}⁴, at each occurrence, are independently selected from the group consisting of absent, hydrogen, halogen, cyano, nitro, hydroxy, amino, optionally substituted C₁-C₆ alkyl, optionally substituted C₁-C₈ heteroalkyl, optionally substituted C₁-C₈ haloalkyl, optionally substituted 3 to 8 membered carbocyclyl, and optionally substituted 3 to 8 membered heterocyclyl; or R_{E}³ and R_{E}⁴, on the same atom or on the adjacent atoms, together with the atom(s) to which they are attached form an optionally substituted 3-8 membered carbocyclyl or heterocyclyl ring.

In some embodiments, the compound is a compound of Formula I wherein, ring A is absent, or an optionally substituted 3-10 membered carbocyclyl, optionally substituted 3-10 membered heterocyclyl, optionally substituted aryl, optionally substituted bicyclic aryl, optionally substituted heteroaryl, or optionally substituted bicyclic heteroaryl; ring B is absent, or an optionally substituted 3-10 membered carbocyclyl, optionally substituted 3-10 membered heterocyclyl, optionally substituted aryl, or optionally substituted heteroaryl; R¹ is absent; R² is hydrogen, halogen, CN, NO₂, OR⁵, SR⁵, N(R⁵)R⁶, C(O)R⁵, C(O)OR⁵, C(O)N(R⁵)R⁶, SO₂R⁵, SO₂N(R⁵)R⁶, NR′C(O)OR⁵, NR⁷C(O)R⁵, NR⁷C(O)N(R⁵)R⁶, NR⁷SOR⁵, NR⁷SO₂R⁵, NR⁷SO₂N(R⁵)R⁶ optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ alkoxy, or optionally substituted C₁-C₈ alkylamino, optionally substituted 3-10 membered carbocyclyl, or optionally substituted 3-10 membered heterocyclyl; X is absent, —O—, or NR⁸; each R⁵, R⁶ and R⁷ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or R⁵ and R⁶ together with the atom(s) to which they are attached optionally form a 3-20 membered heterocyclyl or 5-6 membered heteroaryl ring; R⁸ is hydrogen or C₁-C₈ alkyl; and Linker is a linking moiety connecting the degradation tag to ring B.

In some embodiments, the Degradation Tag is a moiety of FORMULA 5, and the Degradation Tag is connected to the linker moiety of the divalent compound via Z_{E};

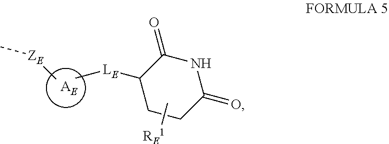

wherein: Z_{E} is a divalent group of —(R_{E}^{z})_{nE}—; wherein subscript n_{E}=0, 1, 2, 3, 4, 5 or 6; wherein R_{E}^{Z}, at each occurrence, is independently R_{E}^{r}, or R_{E}^{w}; wherein R_{E}^{w}, at each occurrence, is absent or selected from the group consisting of —CO—, —CR_{E}⁵R_{E}⁶—, —NR_{E}⁵—, —O—, optionally substituted C₁-C₁₀ alkylene, optionally substituted C₁-C₁₀ alkenylene, optionally substituted C₁-C₁₀ alkynylene; and R_{E}^{r}, at each occurrence, is absent, or selected from the group consisting of optionally substituted 3-10 membered carbocyclyl such as 3-8 membered carbocyclyl, optionally substituted 4-10 membered heterocyclyl such as 3-8 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl; with the proviso that —R_{E}^{z}—R_{E}^{z}— is not —O—O—; R_{E}⁵ and R_{E}⁶ at each occurrence are independently selected from the group consisting of hydrogen, halogen, oxo, hydroxy, amino, cyano, nitro, optionally substituted C₁-C₆ alkyl, optionally substituted C₁-C₈ heteroalkyl, optionally substituted C₁-C₈ haloalkyl, optionally substituted 3 to 8 membered carbocyclyl, and optionally substituted 3 to 8 membered heterocyclyl; or R_{E}⁵ and R_{E}⁶ together with the atom(s) to which they are connected form an optionally substituted 3-8 membered carbocyclyl or heterocyclyl ring; R_{E}¹ is selected from the group consisting of hydrogen, halogen, cyano, nitro, optionally substituted C₁-C₆ alkyl, optionally substituted 3-8 membered carbocyclyl, and optionally substituted 3-8 membered heterocyclyl; L_{E} is a divalent group selected from the group consisting of null, -L_{E}¹-, and -L_{E}¹-L_{E}²-; wherein L_{E}¹ and L_{E}² are independently selected from the group consisting of —CO—, —O—, —CR_{E}¹⁰R_{E}¹¹— and —NR_{E}¹⁰—, with the proviso that -L_{E}¹-L_{E}²- is not —O—O—; wherein R_{E}¹⁰ and R_{E}¹ are independently selected from the group consisting of hydrogen, halogen, cyano, nitro, hydroxy, amino, optionally substituted C₁-C₆ alkyl, optionally substituted C₁-C₆ alkoxy, and optionally substituted C₁-C₆ alkylamino; and Ring A_{E}

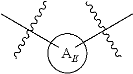

is a divalent group selected from the group consisting of FORMULA A_{E}1, A_{E}2, A_{E}3, A_{E}4 and A_{E}5:

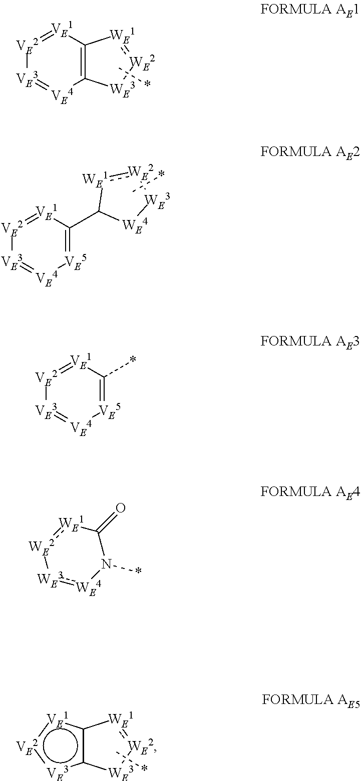

wherein * indicates the attachment to L_{E}, and Z_{E} is attached to any possible position on the Ring A_{E}, [symbol] indicates a single bond or a double bond; V_{E}¹, V_{E}², V_{E}³, V_{E}⁴ and V_{E}⁵, at each occurrence, are each independently selected from the group consisting of absent, C, CR_{E}², S, N, and NR_{E}²; or V_{E}¹ and V_{E}², V_{E}² and V_{E}³, V_{E}³ and V_{E}⁴, or V_{E}⁴ and V_{E}⁵ are combined together to optionally form 6 membered aryl ring or a 5, 6 or 7 membered heteroaryl ring; R_{E}², at each occurrence, is independently selected from the group consisting of absent, hydrogen, halogen, cyano, nitro, hydroxy, amino, optionally substituted C₁-C₆ alkyl, optionally substituted C₁-C₆ alkenyl, optionally substituted C₁-C₆ alkynyl, optionally substituted C₁-C₆ alkoxy, optionally substituted C₁-C₆ alkylamino, optionally substituted 3-8 membered carbocyclyl, and optionally substituted 3-8 membered heterocyclyl; or R_{E}² and another R_{E}² together with the atom(s) to which they are connected form optionally substituted 3-8 membered carbocyclyl, optionally substituted 3-8 membered heterocyclyl ring, optionally substituted aryl, and optionally substituted heteroaryl; W_{E}¹, W_{E}², W_{E}³ and W_{E}⁴ are each independently selected from the group consisting of —N═, —C≡, —CR_{E}³═, —CO—, —O—, —CR_{E}³R_{E}⁴—, —NR_{E}³—, —CR_{E}³═CR_{E}⁴—, —N═CR_{E}³—, and —N═N—; or W_{E}¹ and W_{E}², W_{E}² and W_{E}³, or W_{E}³ and W_{E}⁴ are combined together to optionally form 6 membered aryl ring or a 5, 6 or 7 membered heteroaryl ring; and R_{E}³ and R_{E}⁴, at each occurrence, are independently selected from the group consisting of absent, hydrogen, halogen, cyano, nitro, hydroxy, amino, optionally substituted C₁-C₆ alkyl, optionally substituted C₁-C₈ heteroalkyl, optionally substituted C₁-C₈ haloalkyl, optionally substituted 3 to 8 membered carbocyclyl, and optionally substituted 3 to 8 membered heterocyclyl; or R_{E}³ and R_{E}⁴, on the same atom or on the adjacent atoms, together with the atom(s) to which they are connected form an optionally substituted 3-8 membered carbocyclyl or heterocyclyl ring.

In some embodiments, wherein the moiety of Formula 5 has the structure of Formula 5-1:

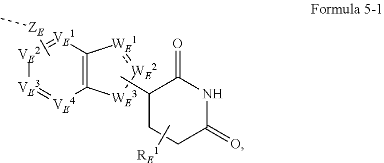

wherein, [symbol] denotes a single bond or a double bond; R_{E}¹ is hydrogen; Z_{E} is absent, —CH₂—, —NH—, or —O—; V_{E}¹, V_{E}², V_{E}³, and V_{E}⁴ are each independently C, CR_{E}², or N; W_{E}¹, W_{E}², and W_{E}³ are each independently selected from the group consisting of —C(O)—, —N—, —NR_{E}³—, or —CR_{E}³R_{E}⁴—; each R_{E}² is independently hydrogen; and R_{E}³ and R_{E}⁴ are each independently selected from the group consisting of hydrogen, halogen, cyano, nitro, hydroxy, amino, or optionally substituted C₁-C₆ alkyl; or R_{E}³ and R_{E}⁴ together with the atom(s) to which they are attached form an optionally substituted 3-6 membered carbocyclyl or 3-6 membered heterocyclyl ring.

In some embodiments the degradation tag comprises structural Formula 5-1 wherein, [symbol] denotes a single bond or a double bond; R_{E}¹ is hydrogen; Z_{E} is absent, —CH₂—, —NH—, or —O—; V_{E}¹, V_{E}², V_{E}³, and V_{E}⁴ are each independently C, CR_{E}², or N; W_{E}¹, W_{E}², and W_{E}³ are each independently selected from the group consisting of —C(O)—, —N—, —NR_{E}³—, or —CR_{E}³R_{E}⁴—; each CR_{E}² is independently hydrogen; and R_{E}³ and R_{E}⁴ are each independently selected from the group consisting of hydrogen, halogen, cyano, nitro, hydroxy, amino, or optionally substituted C₁-C₆ alkyl. In some embodiments, W_{E}¹ and W_{E}³ are each independently C(O) or CH₂; and W_{E}² is N. In some embodiments, the degradation tag has the structure of

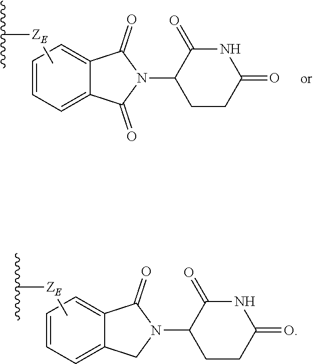

Some embodiments include a linker. In some embodiments, the linker is optional. In some embodiments, the linker moiety comprises structural FORMULA 9:

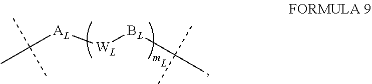

wherein A_{L}, W_{L} and B_{L}, at each occurrence, are independently absent or a divalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ heteroalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl, wherein R_{L}^{d} and R_{L}C are each independently absent or a divalent group selected from an optionally substituted (C₁-C₈ alkylene)-R_{L}^{r}, optionally substituted R_{L}^{r}-(C₁-C₈ alkylene), optionally substituted (C₁-C₈ alkylene)-R_{L}^{r}-(C₁-C₈ alkylene), an optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylamino C₁-C₈alkylene, optionally substituted C₁-C₈ heteroalkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl; R_{L} is selected from optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl; R_{L}¹ and R_{L}² are each independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 3-10 membered heterocyclyl, optionally substituted aryl, and optionally substituted heteroaryl; or R_{L}^{d} and R_{L}^{e}, R_{L}¹ and R_{L}², R_{L}^{d} and R_{L}¹, R_{L}^{d} and R_{L}², R_{L}^{e} and R_{L}¹, or R_{L}^{e} and R_{L}² together with the atom(s) to which they are attached optionally form a 3-20 membered carbocyclyl or 4-20 membered heterocyclyl ring; and m_{L} is 1, 2, 3, 4, 5, 6, 7, 8, 9, 10, 11, 12, 13, 14, or 15.

In some embodiments, the linker comprises structural Formula 9 wherein, A_{L} is an optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, or optionally substituted 3-10 membered heterocyclyl; W_{L} and B_{L}, at each occurrence, are independently absent or a divalent moiety selected from R_{L}^{d}—R_{L}^{e}, R_{L}^{d}C(O)R_{L}^{e}, R_{L}^{d}CO₂R_{L}^{e}, R_{L}^{d}C(O)N(R_{L}¹)R_{L}^{e}, R_{L}^{d}OR_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ heteroalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl; R_{L}^{d} and R_{L}^{e} are each independently absent or divalent group selected from an optionally substituted (C₁-C₈ alkylene)-R_{L}^{r}, optionally substituted R_{L}^{r}-(C₁-C₈ alkylene), optionally substituted (C₁-C₈ alkylene)-R_{L}^{r}-(C₁-C₈ alkylene), optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ heteroalkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl; R_{L} is selected from an optionally substituted aryl or optionally substituted heteroaryl; and m_{L} is 1, 2, or 3.

In some embodiments, A_{L} is an optionally substituted 3-10 membered carbocyclyl or optionally substituted 3-10 membered heterocyclyl; W_{L} and B_{L}, at each occurrence, are independently absent or a divalent moiety selected from R_{L}^{d}—R_{L}^{e}, R_{L}^{d}C(O)R_{L}^{e}, R_{L}^{d}CO₂R_{L}^{e}, R_{L}^{d}C(O)N(R_{L}¹)R_{L}^{e}, R_{L}^{d}OR_{L}^{e}, R_{L}^{d}N(R_{L}¹)R_{L}^{e}, and R_{L}^{d}N(R_{L}¹)COR_{L}^{e}; R_{L}^{d} and R_{L}^{e} are each independently absent or selected from an optionally substituted (C₁-C₈ alkylene)-R_{L}^{r}, optionally substituted R_{L}^{r}-(C₁-C₈ alkylene), optionally substituted (C₁-C₈ alkylene)-R_{L}^{r}-(C₁-C₈ alkylene), optionally substituted C₁-C₈ alkyl, optionally substituted aryl and optionally substituted heteroaryl; and R_{L}^{r} is selected from an optionally substituted aryl, and optionally substituted heteroaryl.

In some embodiments, A_{L} is an optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, or optionally substituted 3-10 membered heterocyclyl; W_{L} and B_{L}, at each occurrence, are independently absent or a divalent moiety selected from R_{L}^{d}—R_{L}^{e}, R_{L}^{d}C(O)R_{L}^{e}, R_{L}^{d}CO₂R_{L}^{e}, R_{L}^{d}C(O)N(R_{L}¹)R_{L}^{e}, R_{L}^{d}OR_{L}^{e}, R_{L}^{d}N(R_{L}J)R_{L}^{e}, R_{L}^{d}N(R_{L}¹)COR_{L}^{e}, R_{L}^{d}N(R_{L}¹)CON(R_{L}²)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ heteroalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl; R_{L}^{d} and R_{L}^{e} are each independently absent or a divalent group selected from an optionally substituted (C₁-C₈ alkylene)-R_{L}^{r}, optionally substituted R_{L}^{r}-(C₁-C₈ alkylene), optionally substituted (C₁-C₈ alkylene)-R_{L}^{r}-(C₁-C₈ alkylene), optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ heteroalkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl; R_{L}^{r} is selected from an optionally substituted aryl, and optionally substituted heteroaryl; and m_{L} is 1, 2, or 3.

In some embodiments, A_{L} is an optionally substituted 5-13 membered spiro carbocyclyl, optionally substituted 5-13 membered spiro heterocyclyl, optionally substituted 3-10 membered carbocyclyl, or optionally substituted 3-10 membered heterocyclyl.

In some embodiments, ring A is absent, or an optionally substituted 3-10 membered carbocyclyl, optionally substituted 3-10 membered heterocyclyl, optionally substituted aryl, optionally substituted bicyclic aryl, optionally substituted heteroaryl, or optionally substituted bicyclic heteroaryl; and ring B is absent, or an optionally substituted 3-10 membered carbocyclyl, optionally substituted 3-10 membered heterocyclyl, optionally substituted aryl, or optionally substituted heteroaryl. In some embodiments, ring A is an optionally substituted 3-10 membered carbocyclyl, optionally substituted 3-10 membered heterocyclyl, optionally substituted aryl, optionally substituted bicyclic aryl, optionally substituted heteroaryl, or optionally substituted bicyclic heteroaryl; and ring B is absent or an optionally substituted heteroaryl.

In some embodiments, ring B is absent or a is a moiety comprising structural Formula B-1:

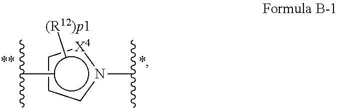

wherein, X⁴ is CR¹² or N; each R¹² is independently hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl; or two R¹² on adjacent carbon atoms join together to form an optionally substituted 3-7 membered carbocyclyl, optionally substituted 4-7 membered heterocyclyl, optionally substituted 6 membered aryl, or optionally substituted 5-6 membered heteroaryl ring; each R⁵ and R is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or R⁵ and R⁶ together with the atom(s) to which they are attached optionally form an optionally substituted 4-7 membered heterocyclyl, or optionally substituted 5-6 membered heteroaryl ring; p₁ is 0, 1, or 2; * denotes the connection to the linker moiety; and ** denotes the connection to X and/or ring A; and ring A is a moiety having the structure of Formula A-1:

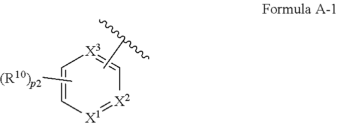

wherein, X¹, X², and X³ are each independently CR¹⁰ or N; each R¹⁰ is independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino; or two R¹⁰ on adjacent carbon atoms join together to form an optionally substituted 3-7 membered carbocyclyl, optionally substituted 4-7 membered heterocyclyl, optionally substituted 6 membered aryl, or optionally substituted 5-6 membered heteroaryl ring; each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or R and R⁶ together with the atom(s) to which they are attached optionally form an optionally substituted 4-7 membered heterocyclyl, or optionally substituted 5-6 membered heteroaryl ring; and p₂ is 0, 1, 2, 3, or 4.

In some embodiments, ring B comprises structural Formula B-2:

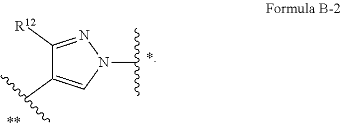

In some embodiments, R¹² is hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted 3-10 membered carbocyclyl, or optionally substituted 3-10 membered heterocyclyl. In some embodiments, R¹² is hydrogen, halogen, CN, OH, CH₃, —OCH₃, CF₃, CF₂, cyclopropyl, cyclobutyl, or cyclopentyl.

In some embodiments, ring A is a moiety comprising structural Formula A-2, Formula A-3, or Formula A-4:

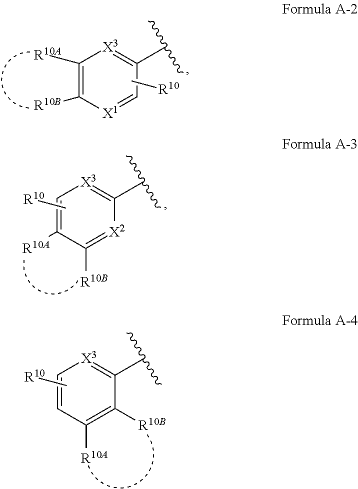

wherein, X¹ is CR¹⁰ or N; X² is CR¹⁰ or N; X³ is CR¹⁰ or N; R^{10A} and R^{10B} are each independently R¹⁰; or R^{10A} and R^{10B} with the atoms to which they are attached join together to form an optionally substituted 6 membered aryl or 5-6 membered heteroaryl ring; each R¹⁰ is independently H, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino; and each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or R⁵ and R⁶ together with the atom(s) to which they are attached optionally form an optionally substituted 4-7 membered heterocyclyl, or optionally substituted 5-6 membered heteroaryl ring.

In some embodiments, ring A comprises structural Formula A-5:

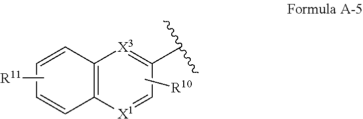

wherein, R¹¹ is hydrogen, halogen, CN, NO₂, OR⁵, SR⁵, N(R⁵)R⁶, C(O)R⁵, C(O)OR⁵, C(O)N(R⁵)R⁶, SO₂R⁵, SO₂N(R⁵)R⁶, NR⁷C(O)OR⁵, NR⁷C(O)R⁵, NR⁷C(O)N(R⁵)R⁶, NR⁷SOR⁵, NR⁷SO₂R⁵, NR⁷SO₂N(R⁵)R⁶ optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈ alkoxy, or optionally substituted C₁-C₈ alkylamino, optionally substituted 3-10 membered carbocyclyl, or optionally substituted 3-10 membered heterocyclyl.

In some embodiment, ring A comprises structural Formula A-6:

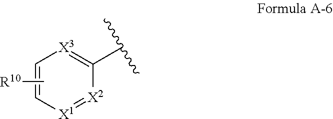

wherein, at least one of X¹, X², or X³ is N. In some embodiments, X³ is N.

In some embodiment, ring A comprises structural Formula A-7:

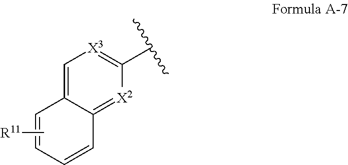

wherein, R¹¹ is hydrogen, halogen, CN, NO₂, OR⁵, SR⁵, N(R⁵)R⁶, C(O)R⁵, C(O)OR⁵, C(O)N(R⁵)R⁶, SO₂R⁵, SO₂N(R⁵)R⁶, NR⁷C(O)OR⁵, NR⁷C(O)R⁵, NR⁷C(O)N(R⁵)R⁶, NR⁷SOR⁵, NR⁷SO₂R⁵, NR⁷SO₂N(R⁵)R⁶ optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl.

In some embodiments, each R¹⁰ is independently hydrogen, halogen, CN, NO₂, OR⁵, N(R⁵)R⁶, C(O)R⁵, C(O)OR⁵, optionally substituted C₁-C₈ alkyl, optionally substituted C₁-C₈ alkoxy, optionally substituted 3-10 membered carbocyclyl, or optionally substituted 3-10 membered heterocyclyl.

In some embodiments, R¹⁰ is hydrogen, halogen, CN, NO₂, OR⁵, N(R⁵)R⁶, C(O)R⁵, C(O)OR⁵, C(O)N(R⁵)R⁶, optionally substituted C₁-C₈ alkyl, optionally substituted C₂-C₈ alkynyl, optionally substituted C₁-C₈ alkoxy, optionally substituted 3-10 membered carbocyclyl, or optionally substituted 3-10 membered heterocyclyl

In one embodiment, R¹ is null. In another embodiment, R¹ is a divalent group comprising a group selected from R′—R″, R′OR″, R′SR″, R′N(R³)R″, R′OC(O)R″, R′OC(O)OR″, R′OCON(R³)R″, R′C(O)R″, R′C(O)OR″, R′CON(R³)R″, R′S(O)R″, R′S(O)₂R″, R′SO₂N(R³)R″, R′NR⁴C(O)OR″, R′NR⁴C(O)R″, R′NR⁴C(O)N(R³)R″, R′NR⁴S(O)R″, R′NR⁴S(O)₂R″, and R′NR⁴S(O)₂NR³R″, wherein R′ and R″ are each divalent groups that are independently selected from null, optionally substituted C₁-C₈ alkylene, optionally substituted C₂-C₈ alkenylene, optionally substituted C₂-C₈ alkynylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl. In a further embodiment, R¹ is R′C(O)R′. In yet another embodiment, R¹ is R′C(O)OR″. In yet a further embodiment, R¹ is C(O).

In one embodiment, R² is selected from hydrogen, halogen, oxo, CN, NO₂, OR⁵, SR⁵, N(R⁵)R⁶, OCOR⁵, OCO₂R⁵, OCON(R⁵)R⁶, COR⁵, CO₂R⁵, CON(R⁵)R⁶, SOR⁵, SO₂R⁵, SO₂N(R⁵)R⁶, NR⁷CO₂R⁵, NR⁷COR⁵, NR⁷C(O)N(R⁵)R⁶, NR⁷SOR⁵, NR⁷SO₂R⁵, NR⁷SO₂N(R⁵)R⁶, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered heterocyclylC₁-C₈alkyl, optionally substituted 3-10 membered carbocyclylC₁-C₈alkyl, optionally substituted 3-10 membered carbocyclyl, optionally substituted 3-10 membered heterocyclyl, optionally substituted aryl, and optionally substituted heteroaryl, wherein R⁵, R⁶, and R⁷ are independently selected from hydrogen, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, and optionally substituted heteroaryl, or R and R⁶, R⁵ and R⁷ together with the atom to which they are connected form a 3-20 membered heterocyclyl ring, or a 5-6 membered heteroaryl.

In another embodiment, R² is selected from hydrogen, halogen, oxo, CN, NO₂, OR⁵, SR⁵, and N(R⁵)R⁶. In yet another embodiment, R² is halogen. In a further embodiment, R² is selected from chloro, bromo, fluoro, and iodo. In one embodiment, R² is optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered heterocyclylC₁-C₈alkyl, optionally substituted 3-10 membered carbocyclylC₁-C₈alkyl, optionally substituted 3-10 membered carbocyclyl, optionally substituted 3-10 membered heterocyclyl, optionally substituted aryl, and optionally substituted heteroaryl. In another embodiment, R² is optionally substituted C₁-C₈ alkyl. In a further embodiment, R² is optionally substituted C₁-C₈ heteroalkyl. In yet a further embodiment, R is methyl, ethyl, n-propyl, iso-propyl, butyl, pentyl, or hexyl. In yet a further embodiment, R² is selected from OH, OCH₃, NH₂, SH, carbocyclyl, heterocyclyl, aryl, or heteroaryl. In a further embodiment, R² is heterocyclyl. In one embodiment, R² is piperidine. In another embodiment, R² is piperidine substituted with C₁-C₈ alkyl. In another embodiment, R² is furan. In yet another embodiment, R² is pyran. In yet a further embodiment, R² is selected from optionally substituted aziridine, pyrrolidine, piperidine, piperazine, morpholine, thiomorpholine, tetrahydrofuran, tetrahydrothiofuran, and tetrahydropyran.

In yet another embodiment, X is selected from null, or a divalent moiety selected from null, CR⁸R⁹, CO, CO₂, CONR⁸, NR⁸, NR⁸CO, NR⁸CO₂, NR⁸C(O)NR⁹, NR⁸SO, NR⁸SO₂, NR⁸SO₂NR⁹, O, OC(O), OCO₂, OCONR⁸, S, SO, SO₂, and SO₂NR⁸, optionally substituted C₁-C₈ alkylene, optionally substituted C₂-C₈ alkenylene, optionally substituted C₂-C₈ alkynylene, optionally substituted 3-10 membered carbocyclyl, optionally substituted 3-10 membered heterocyclyl, optionally substituted aryl, and optionally substituted heteroaryl, wherein R⁸ and R⁹ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈ alkoxy, optionally substituted C₁-C₈ alkylamino, optionally substituted 3-10 membered carbocyclyl, optionally substituted 3-10 membered heterocyclyl, optionally substituted aryl, and optionally substituted heteroaryl, or R⁸ and R⁹ together with the atom or atoms to which they are connected form a 3-20 membered carbocyclyl ring or 3-20 membered heterocyclyl ring. In one embodiment, X is null. In another embodiment, X is selected from CR⁸R⁹, CO, CO₂, CONR⁸, NR⁸, NR⁸CO, NR⁸CO₂, NR⁸C(O)NR⁹, NR⁸SO, NR⁸SO₂, NR⁸SO₂NR⁹, O, OC(O), OCO₂, OCONR⁸, S, SO, SO₂, and SO₂NR⁸. In a further embodiment, X is 0. In some embodiments, X is absent. In yet another embodiment, X is NR⁸ where R⁸ is CH₃. In yet another embodiment, X is an optionally substituted heteroaryl. In yet a further embodiment, the heteroaryl group is selected from pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzotbiazolyl, benzisothiazolyl, benzisoxazolyl, benzoxadiazolyl, benzoxazolyl, cinnolinyl, 1H-indazolyl, 2H-indazolyl, indolizinyl, isobenzofuryl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, and thienoimidazolyl group.

In yet another embodiment, ring A

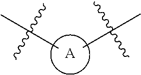

ring B

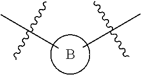

are independently selected from, null, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, optionally substituted bicyclic fused aryl, optionally substituted tricyclic fused aryl, and optionally substituted heteroaryl, optionally substituted bicyclic fused heteroaryl, and optionally substituted tricyclic fused heteroaryl. In one embodiment, ring A is a heterocyclyl. In one embodiment, ring A is piperidine. In another embodiment, ring A is piperidine substituted with C₁-C₈ alkyl. In another embodiment, ring A is furan. In yet another embodiment, ring A is pyran. In yet a further embodiment, ring A is selected from optionally substituted aziridine, pyrrolidine, piperidine, piperazine, morpholine, thiomorpholine, tetrahydrofuran, tetrahydrothiofuran, and tetrahydropyran. In one embodiment, ring B is a heterocyclyl. In one embodiment, ring B is piperidine. In another embodiment, ring B is piperidine substituted with C₁-C₈ alkyl. In another embodiment, ring B is furan. In yet another embodiment, ring B is pyran. In yet a further embodiment, ring B is selected from optionally substituted aziridine, pyrrolidine, pyrrolidine, piperidine, piperazine, morpholine, thiomorpholine, tetrahydrofuran, tetrahydrothiofuran, and tetrahydropyran.

In another embodiment, the Degradation Tag is a moiety of FORMULA 5, and wherein R_{E}² at each occurrence, is independently selected from the group consisting of absent, hydrogen, halogen, cyano, nitro, hydroxy, amino, optionally substituted C₁-C₆ alkyl, optionally substituted C₁-C₆ alkenyl, optionally substituted C₁-C₆ alkynyl, optionally substituted C₁-C₈ heteroalkyl, optionally substituted C₁-C₈ haloalkyl, optionally substituted C₁-C₆ alkoxy, optionally substituted C₁-C₆ alkylamino, optionally substituted 3-8 membered carbocyclyl, optionally substituted 3-8 membered heterocyclyl, optionally substituted aryl, and optionally substituted heteroaryl.

In another embodiment, the Degradation Tag is a moiety of FORMULA 5, and wherein V_{E}¹, V_{E}², V_{E}³, V_{E}⁴ and V_{E}⁵, at each occurrence, are each independently selected from the group consisting of absent, C, CR_{E}², S, N, and NR_{E}²; or V_{E}¹ and V_{E}², V_{E}² and V_{E}³, V_{E}³ and V_{E}⁴, or V_{E}⁴ and V_{E}⁵ are combined to optionally form 6 membered aryl ring or a 5, 6 or 7 membered heteroaryl ring.

In another embodiment, the Degradation Tag is a moiety of FORMULA 5, and wherein Ring A_{E} is a group consisting of FORMULA A_{E}1, and wherein V_{E}¹, V_{E}², V_{E}³, and V_{E}⁴ are each independently selected from the group consisting of C, CR_{E}², S, N, and NR_{E}².

In another embodiment, the Degradation Tag is a moiety of FORMULA 5, and wherein Ring A_{E} is a group consisting of FORMULA A_{E}2, and wherein V_{E}¹, V_{E}², V_{E}³, V_{E}⁴ and V_{E}⁵, at each occurrence, are each independently selected from the group consisting of C, CR_{E}², S, N, and NR_{E}².

In another embodiment, the Degradation Tag is a moiety of FORMULA 5, and wherein Ring A_{E} is a group consisting of FORMULA A_{E}3, and wherein V_{E}¹, V_{E}², V_{E}³, V_{E}⁴ and V_{E}S are each independently selected from the group consisting of C, CR_{E}², S, N, and NR_{E}²; or V_{E}¹ and V_{E}², V_{E}² and V_{E}³, V_{E}³ and V_{E}⁴, or V_{E} and V_{E}⁵ are combined together to optionally form 6 membered aryl ring or a 5, 6 or 7 membered heteroaryl ring.

In another embodiment, the Degradation Tag is a moiety of FORMULA 5, and wherein Ring A_{E} is a group consisting of FORMULA A_{E}4, and wherein [symbol] is a single bond and W_{E}¹, W_{E}², W_{E}³ and W_{E}⁴ are each independently selected from the group consisting of —N═, —CR_{E}³═, —CO—, —O—, —CR_{E}³R_{E}⁴—, and —NR_{E}³.

In another embodiment, the Degradation Tag is a moiety of FORMULA 5, and wherein Ring A_{E} is a group consisting of FORMULA A_{E}5, and wherein V_{E}¹, V_{E}², and V_{E}³ are each independently selected from the group consisting of CR_{E}², S, N, with the proviso that at least one of V_{E}¹, V_{E}², and V_{E}³ is S, N or NR_{E}²; or V_{E}¹ and V_{E}², V_{E}² and V_{E}³ are combined together to optionally form 5 membered heteroaryl ring.

In another embodiment, the Degradation Tag is a moiety of FORMULA 5, and wherein R_{E}¹ is selected from hydrogen, halogen, cyano, nitro, optionally substituted C₁-C₆ alkyl, optionally substituted 3-8 membered carbocyclyl, and optionally substituted 3-8 membered heterocyclyl; preferably, R_{E}¹ is selected from hydrogen, halogen, cyano, nitro, and C₁-C₈ alkyl; more preferably, R_{E}¹ is selected from H, CH₃, or F.

In another embodiment, the Degradation Tag is a moiety of FORMULA 5, and wherein R_{E}² is selected from hydrogen, halogen, cyano, nitro, hydroxy, amino, optionally substituted C₁-C₆ alkyl, optionally substituted C₁-C₆ alkyl, optionally substituted C₁-C₆ alkoxyl, optionally substituted C₁-C₆ alkylamino, optionally substituted 3 to 8 membered carbocyclyl, and optionally substituted 3 to 8 membered heterocyclyl; preferably, R_{E}² is selected from hydrogen, halogen, cyano, nitro, and C₁-C₈ alkyl, optionally substituted C₁-C₆ alkoxyl, optionally substituted 3 to 8 membered carbocyclyl, and optionally substituted 3 to 8 membered heterocyclyl; more preferably, R_{E}² is selected from H, F, OMe, O-iPr, or O-cPr.

In another embodiment, the Degradation Tag is a moiety of FORMULA 5, and wherein R_{E}³ and R_{E}⁴ are independently selected from hydrogen, halogen, cyano, nitro, optionally substituted C₁-C₆ alkyl, optionally substituted 3 to 8 membered carbocyclyl, and optionally substituted 3 to 8 membered heterocyclyl; or R_{E}³ and R_{E}⁴ together with the atom(s) to which they are connected form a 3-8 membered carbocyclyl, or 3-8 membered heterocyclyl.

In another embodiment, the Degradation Tag is a moiety of FORMULA 5, and wherein R_{E}^{r} is selected from Group R_{E}, and Group R_{E} consists of

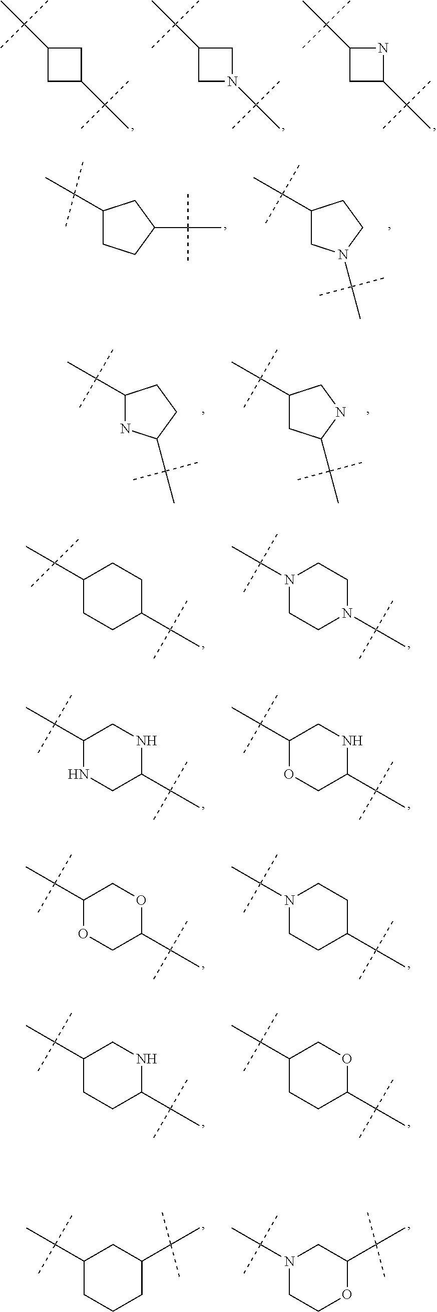

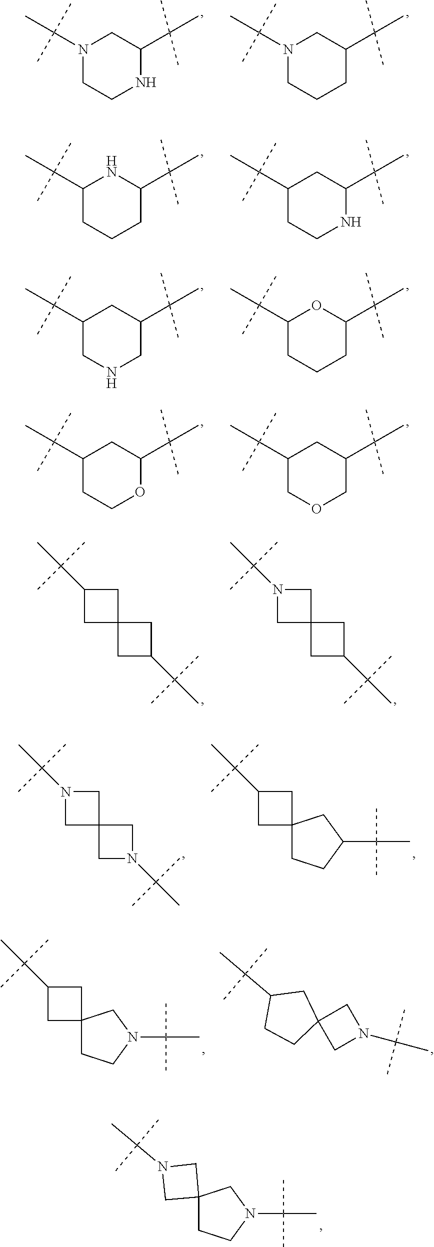

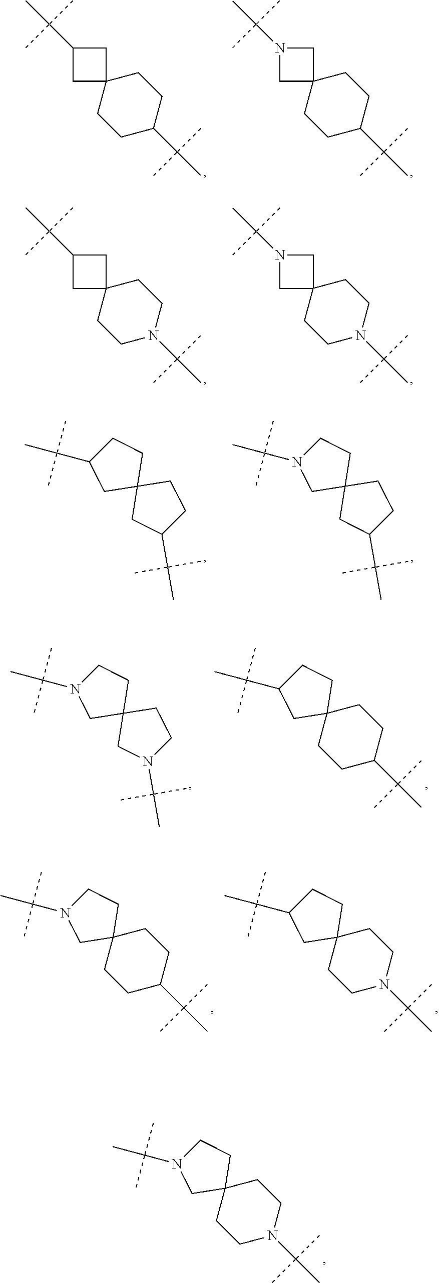

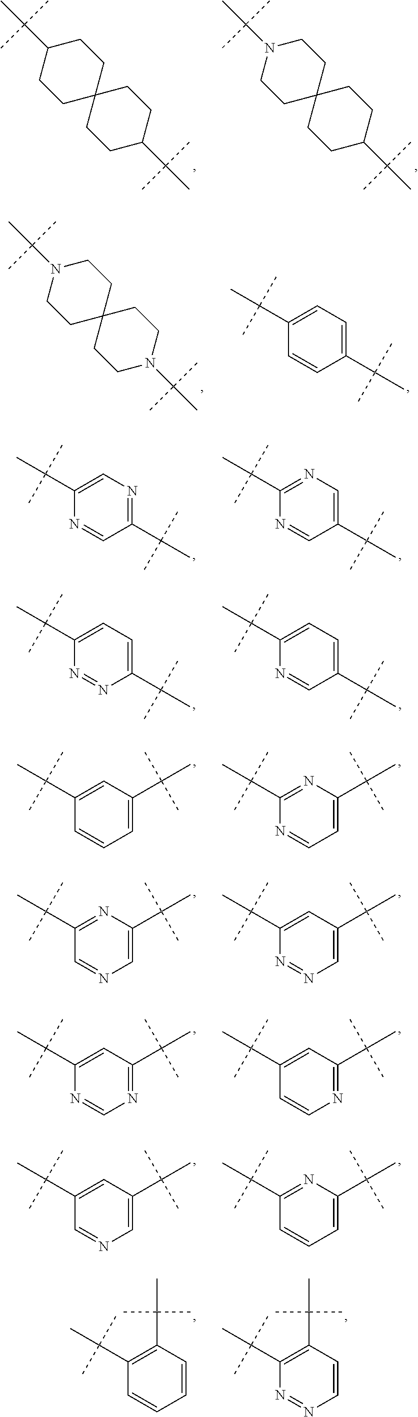

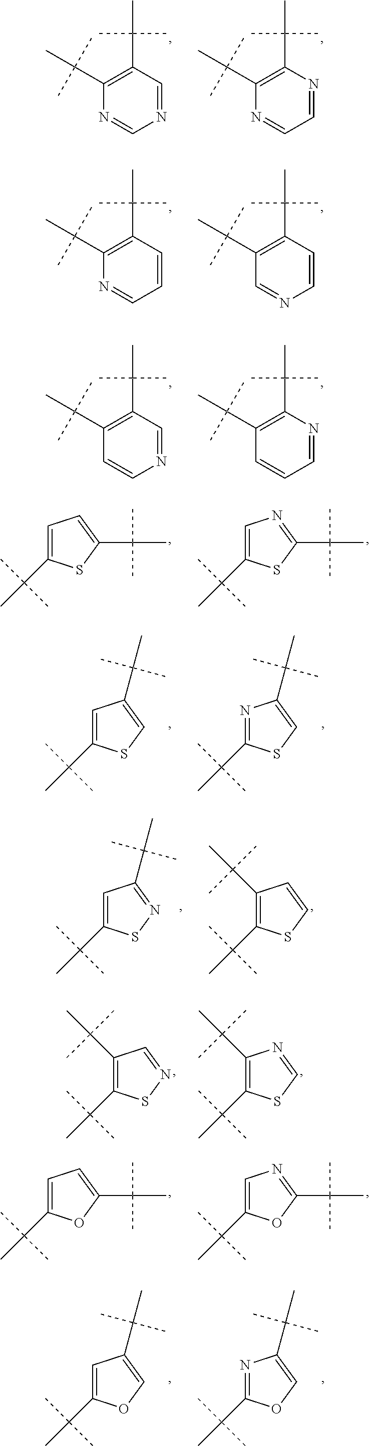

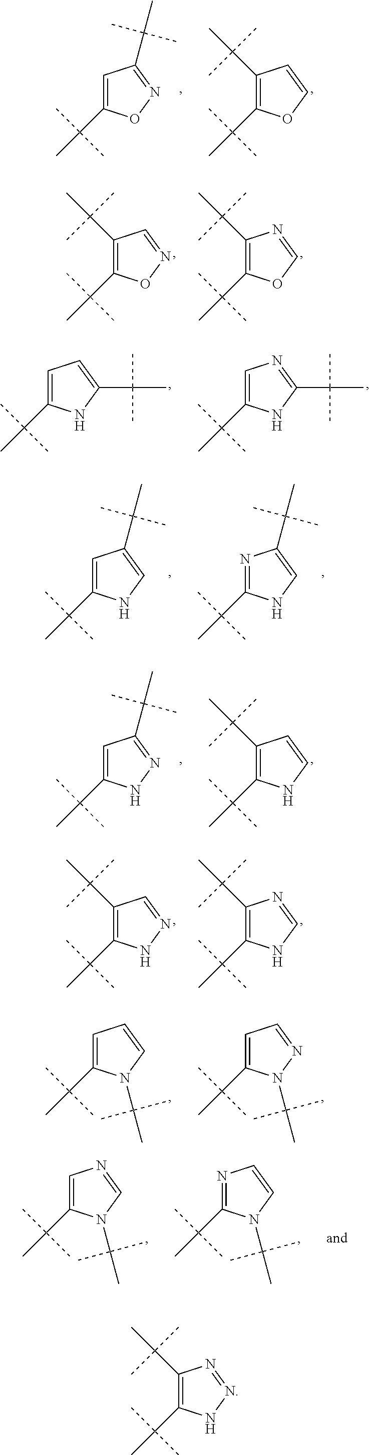

In another embodiment, the Degradation Tag is a moiety of FORMULA 5, and wherein in the group of Z_{E}, at most three R_{E}^{Z} is R_{E}^{r}.

In another embodiment, the Degradation Tag is a moiety of FORMULA 5, and wherein n_{E}=0, 1, 2 or 3.

In another embodiment, the Degradation Tag is a moiety of FORMULA 5, and wherein Z_{E} is a divalent group selected from the group consisting of —R_{E}^{w}—, —(R_{E}^{w})₂—, —(R_{E}^{w})₃—, —R_{E}^{r}—, —R_{E}^{w}—R_{E}^{r}—R_{E}^{w}—, —R_{E}^{r}—R_{E}^{w}— and —R_{E}^{r}—(R_{E}^{w})₂—.

In another embodiment, the Degradation Tag is a moiety of FORMULA 5, and wherein R_{E}⁵ and R_{E}⁶ at each occurrence are independently selected from absent, hydrogen, halogen, oxo, hydroxyl, amino, cyano, nitro, optionally substituted C₁-C₆ alkyl, optionally substituted C₁-C₈ heteroalkyl, optionally substituted C₁-C₈ haloalkyl, optionally substituted 3 to 8 membered carbocyclyl, and optionally substituted 3 to 8 membered heterocyclyl; or R_{E}⁵ and R_{E}⁶ together with the atom(s) to which they are connected form a 3-8 membered carbocyclyl or heterocyclyl ring.

In another embodiment, the Degradation Tag is a moiety of FORMULA 5, and wherein R_{E}^{z} is selected from —CO—, —CR_{E}⁵R_{E}⁶—, —NR_{E}⁵—, —O—, optionally substituted C₁-C₁₀ alkylene, optionally substituted C₁-C₁₀ alkenylene, optionally substituted C₁-C₁₀ alkynylene, optionally substituted 3-8 membered carbocyclyl, optionally substituted 3-8 membered heterocyclyl.

In another embodiment, the Degradation Tag is a moiety of FORMULA 5, and wherein Z_{E} is selected from absent, CH₂, CH═CH, C≡C, NH, and O.

In another embodiment, the Degradation Tag is a moiety of FORMULA 5, and wherein Ring A_{E} is of FORMULA A_{E}4 and L_{E} is not null.

In another embodiment, the Degradation Tag is a moiety of FORMULA 5, and wherein Ring A_{E} is of FORMULA A_{E}4 and L_{E} is selected from the group consisting of —NH—, —N(C₁-C₄ alkyl)-, —CO—, —NH—CO—, —N(C₁-C₄ alkyl)-CO—, —CO—NH—, and —CO—N(C₁-C₄ alkyl)-.

In another embodiment, the Degradation Tag is a moiety selected from the group consisting of FORMULA 5-1, 5-2, 5-3, 5-4, 5-5 and 5-6, and the Degradation Tag is connected to the linker moiety of the divalent compound via a divalent group of Z_{E};

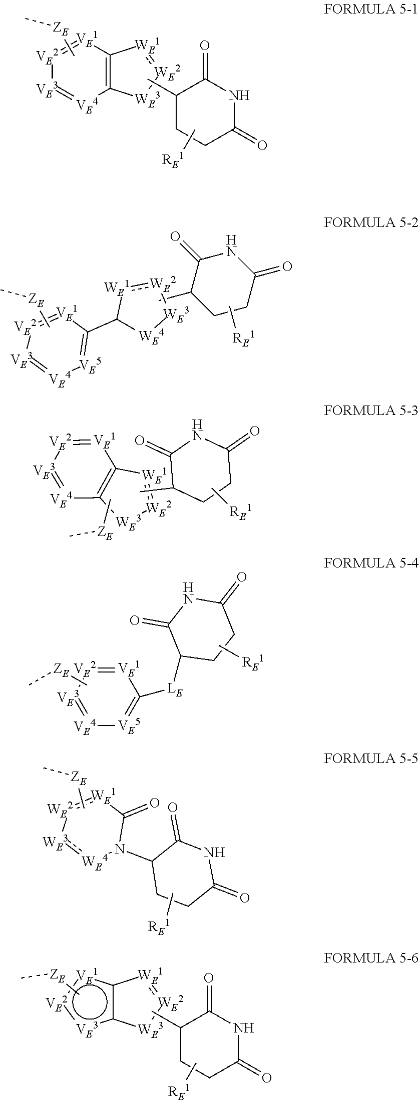

wherein
Z_{E}, R_{E}¹. L_{E}, [symbol] , V_{E}¹, V_{E}², V_{E}³, V_{E}⁴, V_{E}⁵, W_{E}¹, W_{E}², W_{E}³ and W_{E}⁴ are defined as in FORMULA 5.

In another embodiment, the Degradation Tag is a moiety selected from the group consisting of FORMULAE 5A, 5B, 5C, 5D, 5E, 5F, 5G, 5H, 5I, 5J, 5K, 5L and 5M:

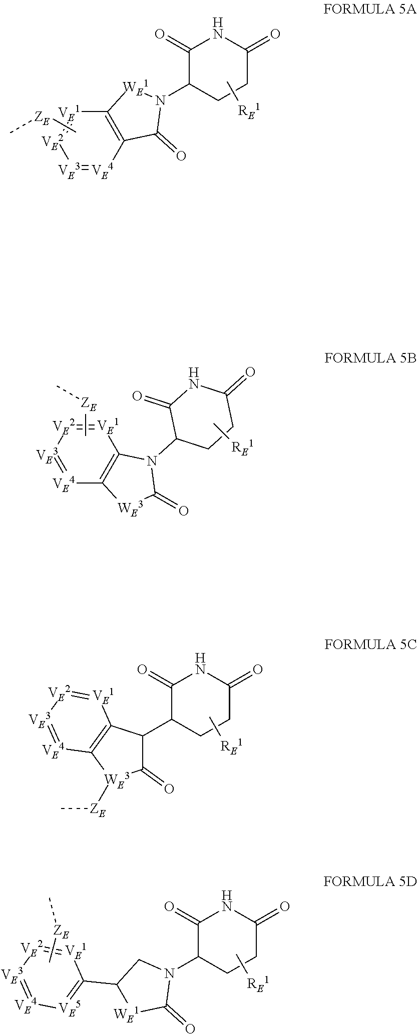

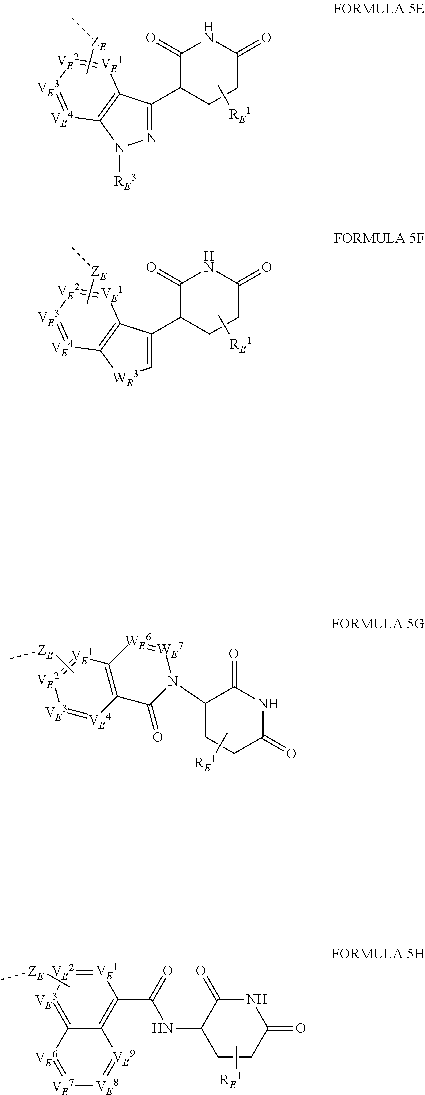

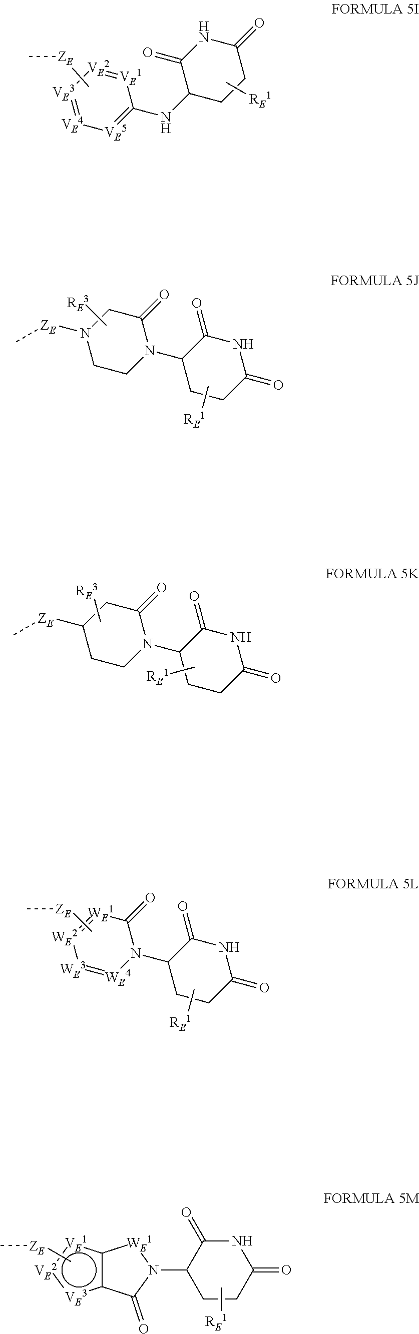

wherein,
V_{E}⁶, V_{E}⁷, V_{E}⁸, and V_{E}⁹ are each independently selected from absent, C, CR_{E}¹² and N; or V_{E}¹ and V_{E}², V_{E}² and V_{E}³, V_{E}³ and V_{E}⁴, or V_{E}⁴ and V_{E}⁵ are combined together to optionally form 6 membered aryl ring or a 5, 6 or 7 membered heteroaryl ring;
R_{E}¹², at each occurrence, is independently selected from the group consisting of hydrogen, halogen, cyano, nitro, hydroxy, amino, optionally substituted C₁-C₆ alkyl, optionally substituted C₁-C₆ alkenyl, optionally substituted C₁-C₆ alkynyl, optionally substituted C₁-C₆ alkoxy, optionally substituted C₁-C₆ alkylamino, optionally substituted 3-8 membered carbocyclyl, and optionally substituted 3-8 membered heterocyclyl;
W_{E}⁶ and W_{E}⁷ are each independently selected from —CR_{E}²═ and —N═;
W_{E}¹, W_{E}², W_{E}³, W_{E}⁴, V_{E}¹, V_{E}², V_{E}³, V_{E}⁴, V_{E}⁵, R_{E}¹, R_{E}³, and Z_{E} are defined as in FORMULA 5.

In another embodiment, W_{E}¹ is selected from —CO—, —O—, —CR_{E}³R_{E}⁴—, —NR_{E}³—, —CR_{E}³═CR_{E}⁴—, —N═CR_{E}³—, and —N═N—.

In another embodiment, the Degradation Tag is a moiety of FORMULA 5-1, or FORMULA 5-3,

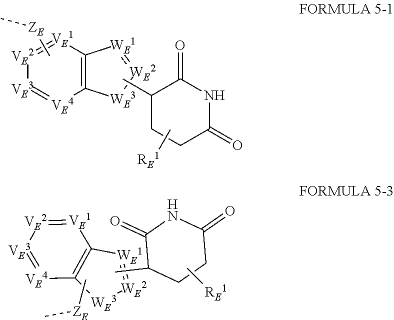

wherein
V_{E}¹, V_{E}², V_{E}³, and V_{E}⁴ are each independently selected from absent, C, CR_{E}², and N; or V_{E}¹ and V_{E}², V_{E}² and V_{E}³, or V_{E}³ and V_{E}⁴ are combined together to optionally form 6 membered aryl ring, or 5, 6 or 7 membered heteroaryl ring;
[symbol] indicates a single bond or a double bond; wherein (i) when there is a single bond between W_{E}¹ and W_{E}² (i.e. the [symbol] between W_{E}¹ and W_{E}² indicates single bond), W_{E}¹, W_{E}² and W_{E}³ are each independently selected from the group consisting of —N═, —CR_{E}³=, —CO—, —O—, —CR_{E}³R_{E}⁴—, —NR_{E}³—, —CR_{E}³═CR_{E}⁴—, —N═CR_{E}³—, and —N═N—; or (ii) when there is a double bond between W_{E}¹ and W_{E}² (i.e. the [symbol] between W_{E}¹ and W_{E}² indicates a double bond), W_{E}¹ and W_{E}² are each independently selected from the group consisting of —N═, —C— and —CR_{E}³—; W_{E}³ is selected from the group consisting of —O—, —N—, —NR_{E}³—, —C(O)NR_{E}³—, —CR_{E}³═CR_{E}⁴—, and —CR_{E}³═N—; Z_{E}, R_{E}², R_{E}³, R_{E}⁴ and R_{E}¹ are defined as in FORMULA 5.

In another embodiment, the Degradation Tag is a moiety of FORMULA 5-1 or 5-3, and wherein V_{E}¹, V_{E}², V_{E}³, and V_{E}⁴ are each independently selected from C, N, and CR_{E}².

In another embodiment, the Degradation Tag FORMULA 5-1 is moiety of FORMULA 5A, 5B, 5E, 5F or 5G

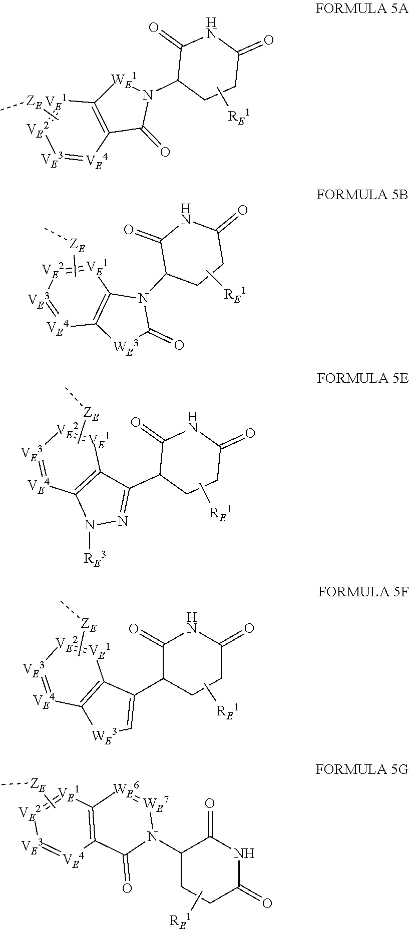

wherein W_{E}⁶ and W_{E}⁷ are each independently selected from —CR_{E}²— and —N═; and V_{E}¹, V_{E}², V_{E}³, V_{E}⁴, W_{E}¹, W_{E}³, Z_{E}, R_{E}³ and R_{E}¹ are defined as in FORMULA 5-1.

In another embodiment, the Degradation Tag is a moiety of FORMULA 5A, 5B, 5E, 5F or 5G, and wherein V_{E}¹, V_{E}², V_{E}³, and V_{E}⁴ are each independently selected from absent, C, CR_{E}² and N (preferably, C, CR_{E}² and N).

In another embodiment, the Degradation Tag is a moiety of FORMULA 5A, 5B, 5E, 5F or 5G, and wherein W_{E}¹ and W_{E}³ are each independently selected from —CO—, —O—, —CR_{E}³R_{E}⁴—, —NR_{E}³—, —CR_{E}³═CR_{E}⁴—, —N═CR_{E}³—, and —N═N—; preferably, W_{E} and W_{E}³ are each independently selected from —CO—, —O—, —CR_{E}³R_{E}⁴—, and —NR_{E}³—.

In another embodiment, the Degradation Tag FORMULA 5-3 is moiety of FORMULA 5C

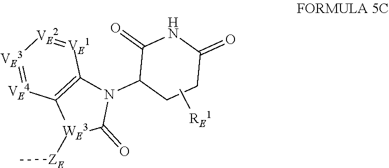

wherein W_{E}³ is N or CR_{E}³; and V_{E}¹, V_{E}², V_{E}³, V_{E}⁴, Z_{E}, and R_{E}¹ are defined as in FORMULA 5-3. In another embodiment, the Degradation Tag is a moiety of FORMULA 5C, wherein V_{E}¹, V_{E}², V_{E}³, and V_{E}⁴ are each independently selected from absent, CR_{E}² and N.

In another embodiment, the Degradation Tag is a moiety of FORMULA 5-2,

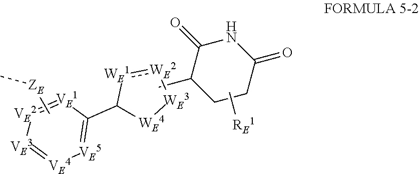

V_{E}¹, V_{E}², V_{E}³, V_{E}⁴ and V_{E}⁵ are each independently selected from absent, C, CR_{E}², and N; or V_{E}¹ and V_{E}², V_{E}² and V_{E}³, V_{E}³ and V_{E}⁴, or V_{E}⁴ and V_{E}⁵ are combined together to optionally form 6 membered aryl ring or 5, 6, or 7 heteroaryl ring;

[symbol] indicates a single bond or a double bond; (i) when there is a single bond between W_{E}¹ and W_{E}² (i.e. the between W_{E}¹ and W_{E}² indicates single bond), W_{E}¹ and W_{E}⁴ are each independently selected from —N═, —CR_{E}³=, —CO—, —O—, —CR_{E}³R_{E}⁴—, —NR_{E}³—, —CR_{E}³═CR_{E}⁴—, —N═CR_{E}³—, and —N═N—, and W_{E}² and W_{E}³ are each independently selected from —N═, —CR_{E}³=, —CO—, —O—, —CR_{E}³R_{E}⁴—, and —NR_{E}³—; or (ii) when there is a double bond between W_{E}¹ and W_{E}² (i.e. the [symbol] between W_{E}¹ and W_{E}² indicates a double bond), W_{E}¹ and W_{E}² are each independently selected from —N═, C and —CR_{E}²=; W_{E}³ is selected from —N═, —CR_{E}³=, —CO—, —O—, —CR_{E}³R_{E}⁴—, and —NR_{E}³—; and W_{E}⁴ is selected from —N═, —CR_{E}³=, —CO—, —O—, —CR_{E}³R_{E}⁴—, —NR_{E}³—, —CR_{E}³═CR_{E}⁴—, —N═CR_{E}³—, and —N═N—; Z_{E}, R_{E}², R_{E}³, R_{E}⁴ and R_{E}¹ are defined as in FORMULA 5.

In another embodiment, the Degradation Tag is a moiety of FORMULA 5-2, wherein V_{E}¹, V_{E}², V_{E}³, V_{E}⁴ and V_{E}⁵ are each independently selected from absent, C, CR_{E}², and N.

In another embodiment, the Degradation Tag is a moiety of FORMULA 5-2, wherein [symbol] indicates a single bond.

In another embodiment, the Degradation Tag is a moiety of FORMULA 5-2, wherein [symbol] indicates a single bond, W_{E}¹ and W_{E}⁴ are each independently selected from —CO—, —O—, —CR_{E}³R_{E}⁴—, and —NR_{E}³—, and W_{E}² and W_{E}³ are each independently selected from —N═, —CR_{E}³=, —CO—, —O—, —CR_{E}³R_{E}⁴—, and —NR_{E}³—.

In another embodiment, the Degradation Tag FORMULA 5-2 is a moiety of FORMULA 5D.

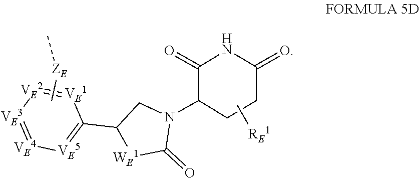

wherein V_{E}¹, V_{E}², V_{E}³, V_{E}⁴, V_{E}⁵, W_{E}¹, Z_{E}, and R_{E}¹ are defined as in FORMULA 5-2.

In another embodiment, the Degradation Tag is a moiety of FORMULA 5D, wherein W_{E}¹ is selected from —CO—, —O—, —CR_{E}³R_{E}¹—, —NR_{E}³—, —CR_{E}³═CR_{E}⁴—, —N═CR_{E}³—, and —N═N—; preferably, W_{E}¹ is seleted from —CO—, —O—, —CR_{E}³R_{E}⁴—, and —NR_{E}³—.

In another embodiment, the Degradation Tag is a moiety of FORMULA 5D, wherein V_{E}¹, V_{E}², V_{E}³, V_{E}⁴, and V_{E}S are each independently selected from absent, C, CR_{E}² and N; or V_{E}¹ and V_{E}², V_{E}² and V_{E}³, V_{E}³ and V_{E}⁴, or V_{E}⁴ and V_{E}⁵ are combined together to optionally form a 6 membered aryl ring or 5, 6 or 7 heteroaryl ring; preferably, V_{E}¹, V_{E}², V_{E}³, V_{E}⁴, and V_{E}⁵ are each independently selected from absent, C, CR_{E}² and N.

In another embodiment, the Degradation Tag is a moiety of FORMULA 5-4,

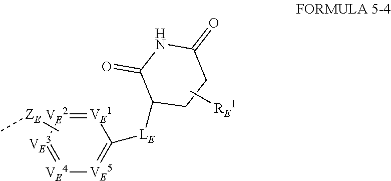

wherein V_{E}¹, V_{E}², V_{E}³, V_{E}⁴, V_{E}⁵. L_{E}, Z_{E}, and R_{E}¹ are defined as in FORMULA 5.

In another embodiment, the Degradation Tag is a moiety of FORMULA 5-4, and wherein L_{E} is not null.

In another embodiment, the Degradation Tag is a moiety of FORMULA 5-4, and wherein L_{E} is selected from the group consisting of —NH—, —N(C₁-C₄ alkyl)-, —CO—, —NH—CO—, —N(C₁-C₄ alkyl)-CO—, —CO—NH—, and —CO—N(C₁-C₄ alkyl)-.

In another embodiment, the Degradation Tag is a moiety of FORMULA 5-4, and wherein
V_{E}¹, V_{E}², V_{E}³, V_{E}⁴ and V_{E}⁵, at each occurrence, are each independently selected from the group consisting of C, CR_{E}² and N; or
V_{E}¹ and V_{E}², V_{E}³ and V_{E}³, V_{E}³ and V_{E}⁴; or V_{E}⁴ and V_{E}⁵ are combined together to optionally form a ring of

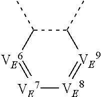

wherein V_{E}⁶, V_{E}⁷, V_{E}⁸, and V_{E}⁹ are each independently selected from the group consisting of C, CR_{E}¹² and N;
R_{E}12, at each occurrence, is independently selected from the group consisting of hydrogen, halogen, cyano, nitro, hydroxy, amino, optionally substituted C₁-C₆ alkyl, optionally substituted C₁-C₆ alkenyl, optionally substituted C₁-C₆ alkynyl, optionally substituted C₁-C₆ alkoxy, optionally substituted C₁-C₆ alkylamino, optionally substituted 3-8 membered carbocyclyl, and optionally substituted 3-8 membered heterocyclyl.

In another embodiment, the Degradation Tag is a moiety of FORMULA 5-4, and wherein V_{E}⁶, V_{E}⁷, V_{E}⁸, and V_{E}⁹ are each independently selected from the group consisting of CR_{E}¹² and N.

In another embodiment, the Degradation Tag is a moiety of FORMULA 5-4, and wherein R_{E}12 at each occurrence, is independently selected from the group consisting of hydrogen, halogen, cyano, nitro, hydroxy, amino, optionally substituted C₁-C₆ alkyl.

In another embodiment, the Degradation Tag is a moiety of FORMULA 5-4, and wherein

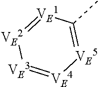

is selected from the group consisting of

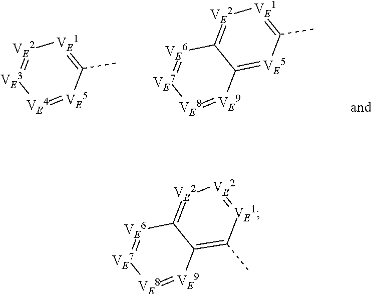

wherein
V_{E}¹, V_{E}², V_{E}³, V_{E}⁴ and V_{E}⁵ are each independently selected from the group consisting of C, CR_{E}² and N; and V_{E}⁶, V_{E}⁷, V_{E}⁸, and V_{E}⁹ are each independently selected from the group consisting of CR_{E}¹² and N.

In another embodiment, the Degradation Tag is a moiety of FORMULA 5-4, and wherein Z_{E} is null, —CH—, —O—, or —NH—.

In another embodiment, the Degradation Tag FORMULA 5-4 is moiety of FORMULA 5H, or 5I;

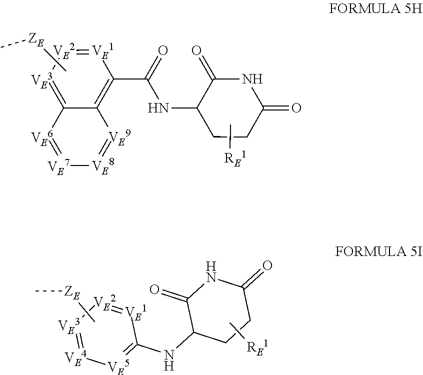

wherein V_{E}¹, V_{E}², V_{E}³, V_{E}⁴, V_{E}⁵, V_{E}⁶, V_{E}¹, V_{E}⁸, and V_{E}⁹ are each independently selected from absent, C, CR_{E}² and N; and Z_{E} and R_{E}¹ are defined as in FORMULA 5-4.

In another embodiment, the Degradation Tag is a moiety of FORMULA 5-5,

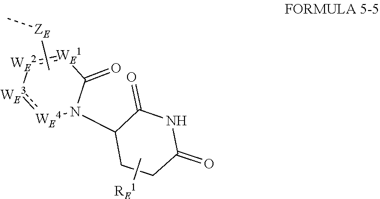

wherein [symbol] W_{E}¹, W_{E}², W_{E}³, W_{E}⁴, Z_{E}¹ and R_{E}¹ are defined as in FORMULA 5.

In another embodiment, the Degradation Tag is a moiety of FORMULA 5-5, and wherein W_{E}¹, W_{E}², W_{E}³ and W_{E}⁴ are each independently selected from the group consisting of —N═, —C≡, —CR_{E}³=, —CO—, —O—, —CR_{E}³R_{E}⁴—, and —NR_{E}³—.

In another embodiment, the Degradation Tag is a moiety of FORMULA 5-5, and wherein W_{E}¹, W_{E}², W_{E}³ and W_{E} are each independently selected from the group consisting of —N═, —C≡, —CH≡, —CO—, —O—, —N—, —CH—, and —NH—.

In another embodiment, the Degradation Tag FORMULA 5-5 is moiety of FORMULA 5J, 5K or 5L;

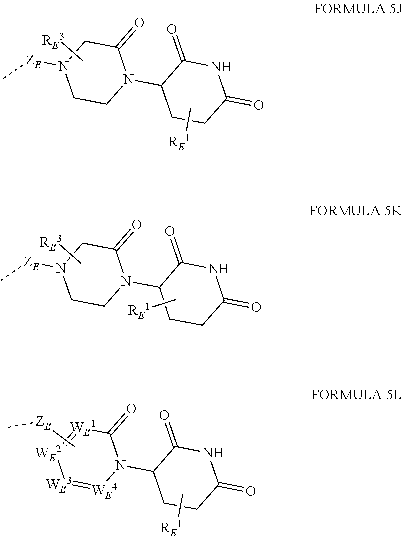

wherein W_{E}¹, W_{E}², W_{E}³, W_{E}⁴, Z_{E}, R_{E}³ and R_{E}¹ are defined as in FORMULA 5-5.

In another embodiment, the Degradation Tag is a moiety of FORMULA 5-6,

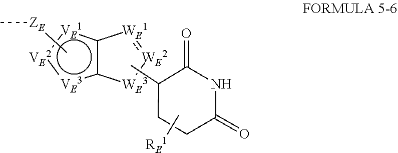

wherein
V_{E}¹, V_{E}², and V_{E}³ are each independently selected from C, CR_{E}², S, N, and NR_{E}²; or V_{E}¹ and V_{E}², or V_{E}² and V_{E}³ are c n d to ether to optionally form 5 membered heteroaryl ring;

[symbol] indicates a single bond or a double bond; wherein (i) when there is a single bond between W_{E}¹ and W_{E}² (i.e. the [symbol] between W_{E}¹ and W_{E}² indicates single bond), W_{E}¹, W_{E}² and W_{E}³ are each independently selected from the group consisting of —N═, —CR_{E}³=, —CO—, —O—, —CR_{E}³R_{E}⁴—, —NR_{E}³—, —CR_{E}³═CR_{E}⁴—, —N═CR_{E}³—, and —N═N—; or (ii) when there is a double bond between W_{E}¹ and W_{E}² (i.e. the [symbol] between W_{E}¹ and W_{E}² indicates a double bond), W_{E}¹ and W_{E}² are each independently selected from the group consisting of —N═, —C≡ and —CR_{E}³═; W_{E}³ is selected from the group consisting of —O—, —N═, —NR_{E}³—, —C(O)NR_{E}³—, —CR_{E}³═CR_{E}⁴—, and —CR_{E}³═N—; and
Z_{E}, R_{E}², R_{E}³, R_{E}⁴ and R_{E}¹ are defined as in FORMULA 5.

In another embodiment, the Degradation Tag is a moiety of FORMULA 5-6, and wherein V_{E}¹, V_{E}², V_{E}³, and V_{E}⁴ are each independently selected from C, CR_{E}², S, N, and NR_{E}².

In another embodiment, the Degradation Tag FORMULA 5-6 is moiety of FORMULA 5M:

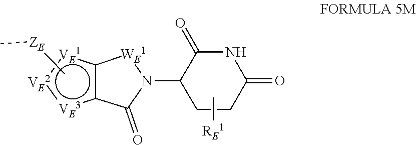

wherein V_{E}¹, V_{E}², V_{E}³, W_{E}¹, Z_{E} and R_{E}¹ are defined as in FORMULA 5-6.

In another embodiment, the Degradation Tag is a moiety of FORMULA 5M, and wherein V_{E}¹, V_{E}², and V_{E}³ are each independently selected from C, CR_{E}², S, N, and NR_{E}² (preferably, one of V_{E}¹, V_{E}², and V_{E}³ is S).

In another embodiment, the Degradation Tag is a moiety of FORMULA 5M, and wherein W_{E}¹ is selected from —CO—, —O—, —CR_{E}³R_{E}⁴—, —NR_{E}³—, —CR_{E}³═CR_{E}⁴—, —N═CR_{E}³—, and —N═N—; preferably, W_{E}¹ is selected from —CO—, —O—, —CR_{E}³R_{E}⁴—, and —NR_{E}³—.

In another embodiment, the Degradation Tag is a moiety of FORMULA 5-1.

In another embodiment, the Degradation Tag is a moiety of FORMULA 5-6.

In another embodiment, the Degradation Tag is a moiety of FORMULA 5A.

In another embodiment, the Degradation Tag is a moiety of FORMULA 5M.

In another embodiment, the Degradation Tag is derived from any of the following:

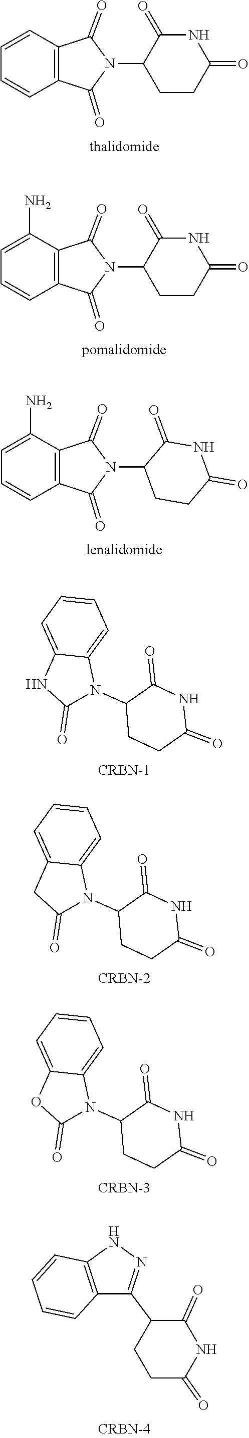

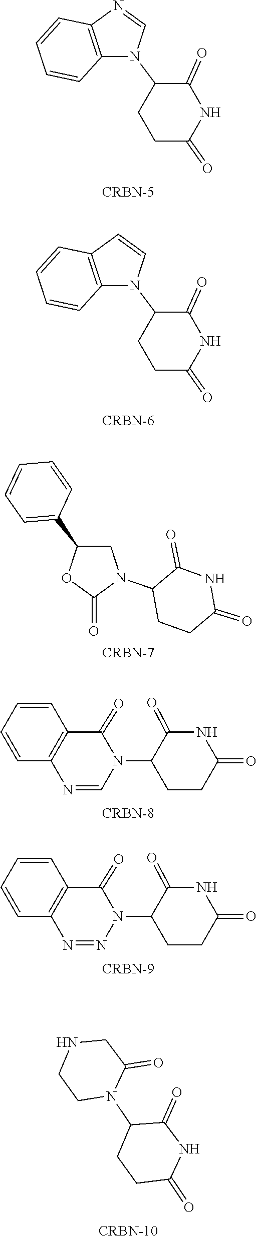 [SMILES: O=C1CCC(n2cnc3ccccc32)C(=O)N1 ; O=C1CCC(n2ccc3ccccc32)C(=O)N1 ; O=C1CCC(N2C[C@H](c3ccccc3)OC2=O)C(=O)N1 ; O=C1CCC(n2cnc3ccccc3c2=O)C(=O)N1 ; O=C1CCC(n2nnc3ccccc3c2=O)C(=O)N1 ; O=C1CCC(N2CCNCC2=O)C(=O)N1]

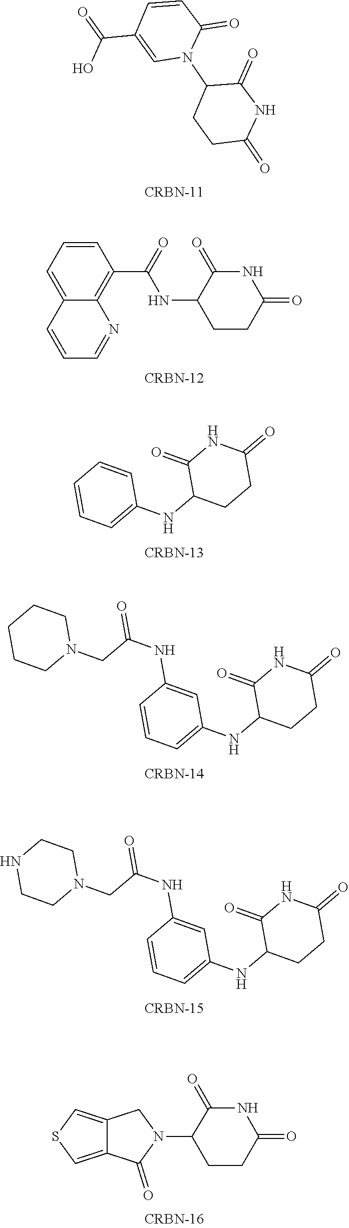 [SMILES: O=C1CCC(n2cc(C(=O)O)ccc2=O)C(=O)N1 ; O=C1CCC(NC(=O)c2cccc3cccnc23)C(=O)N1 ; O=C1CCC(Nc2ccccc2)C(=O)N1 ; O=C1CCC(Nc2cccc(NC(=O)CN3CCCCC3)c2)C(=O)N1 ; O=C1CCC(Nc2cccc(NC(=O)CN3CCNCC3)c2)C(=O)N1 ; O=C1CCC(N2Cc3cscc3C2=O)C(=O)N1]

In another embodiment, the Degradation Tag is derived from any of thalidomide, pomalidomide, lenalidomide, CRBN-1, CRBN-2, CRBN-3, CRBN-4, CRBN-5, CRBN-6, CRBN-7, CRBN-8, CRBN-9, CRBN-10, CRBN-11, CRBN-12, CRBN-13, CRBN-14, CRBN-15, and CRBN-16.

In another embodiment, the Degradation Tag is derived from any of the following FORMULAE:

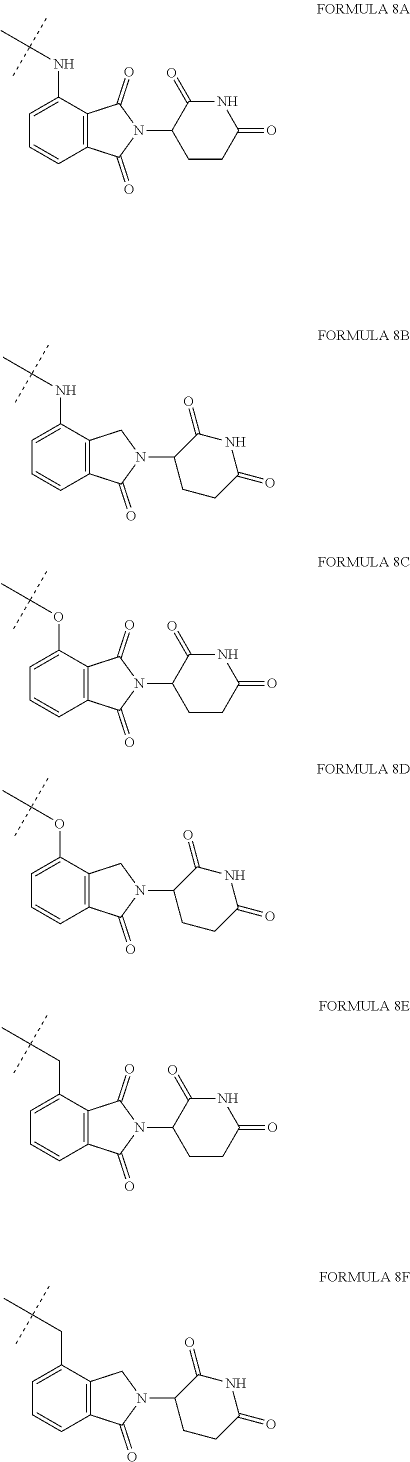

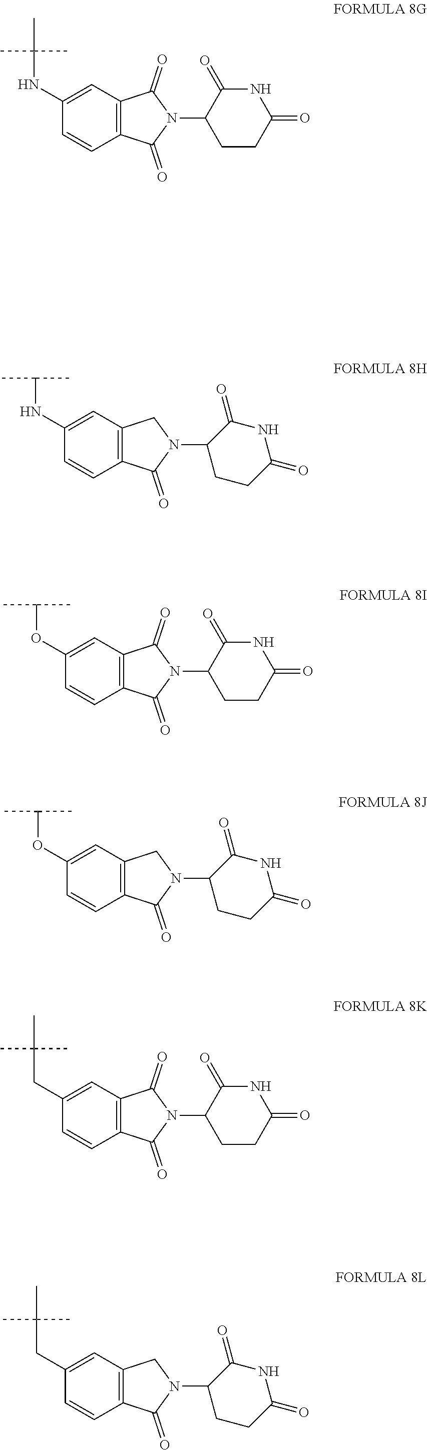

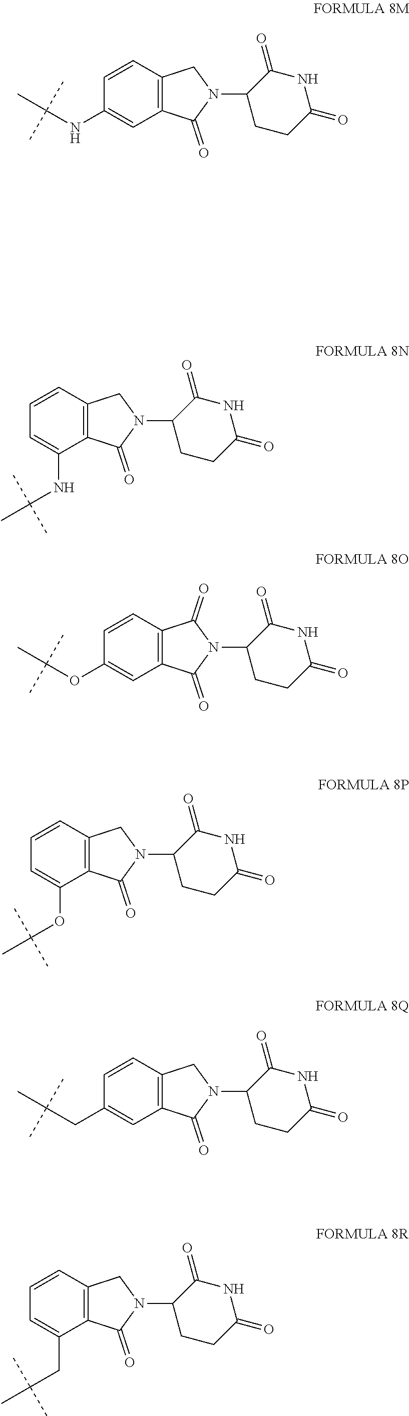

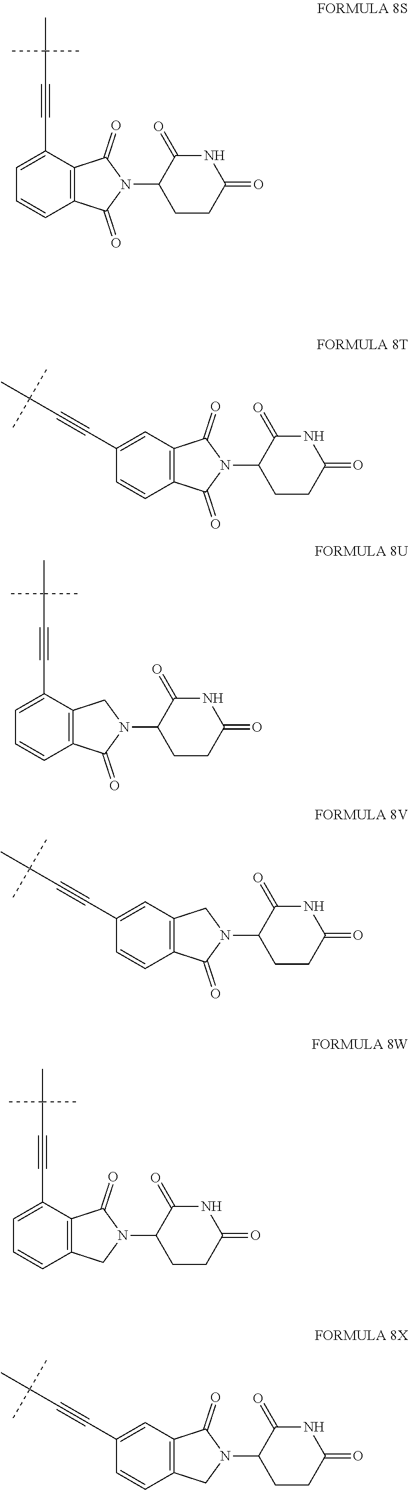

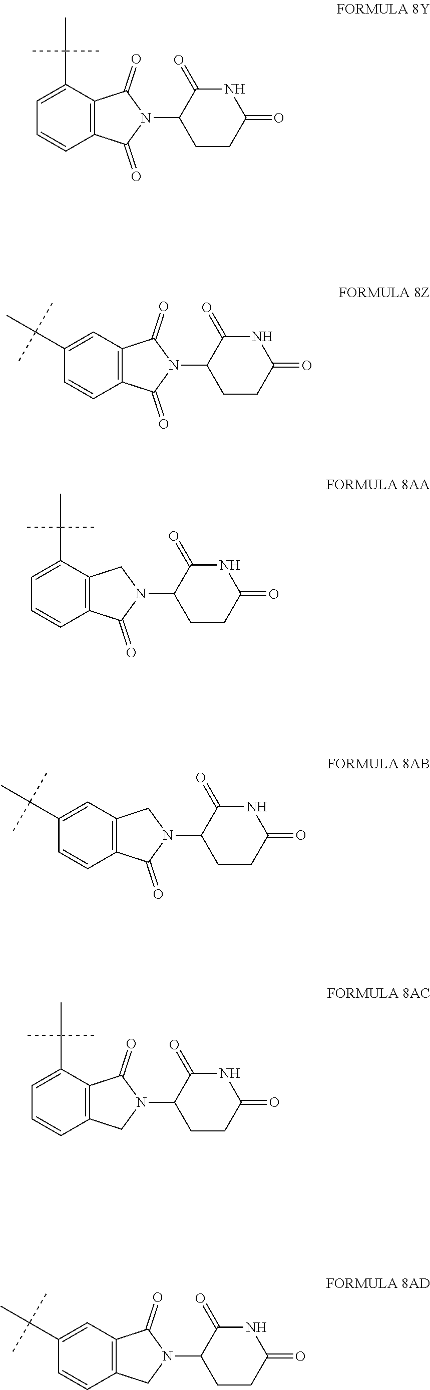

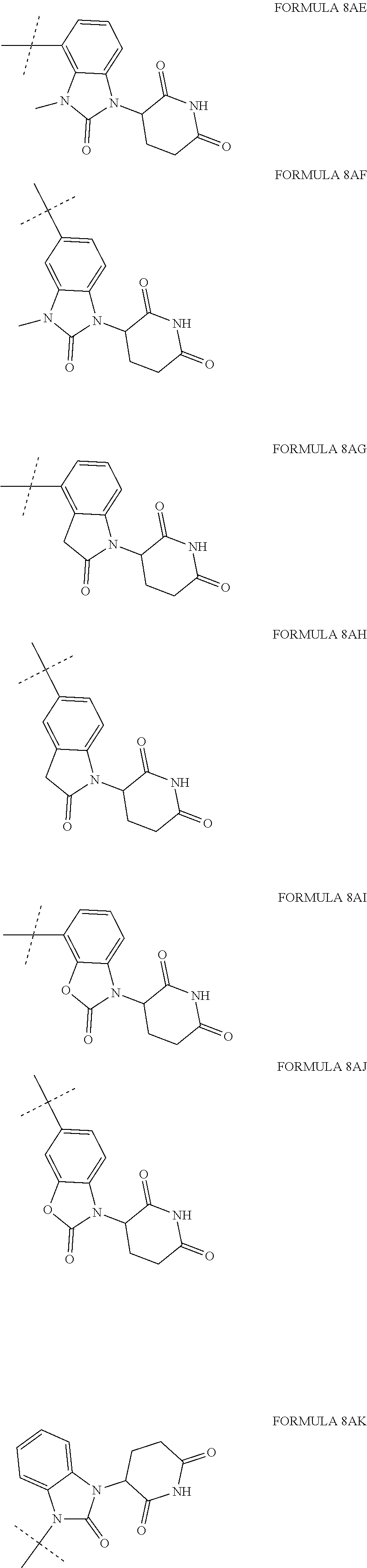

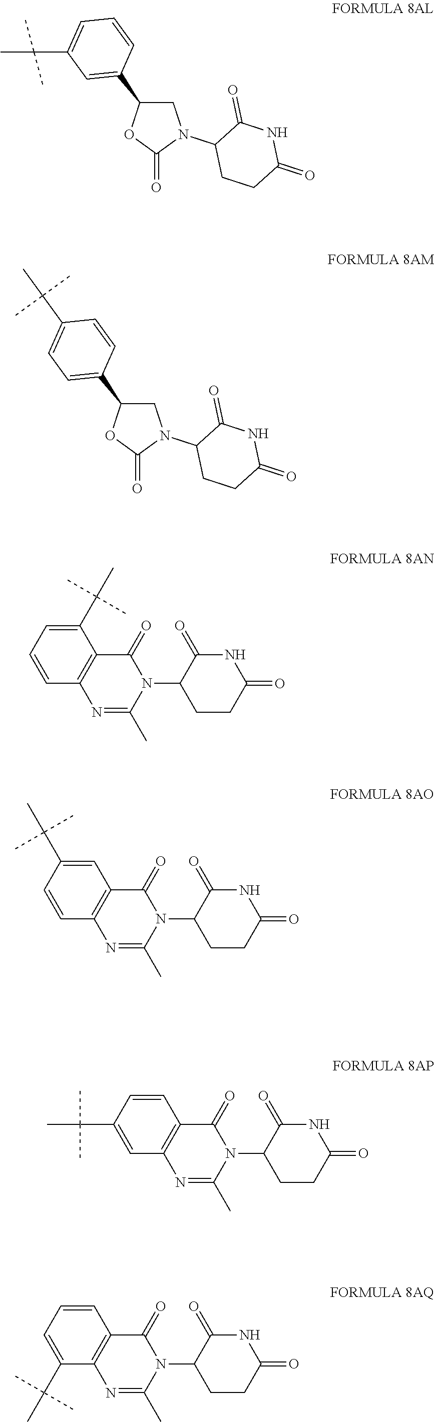

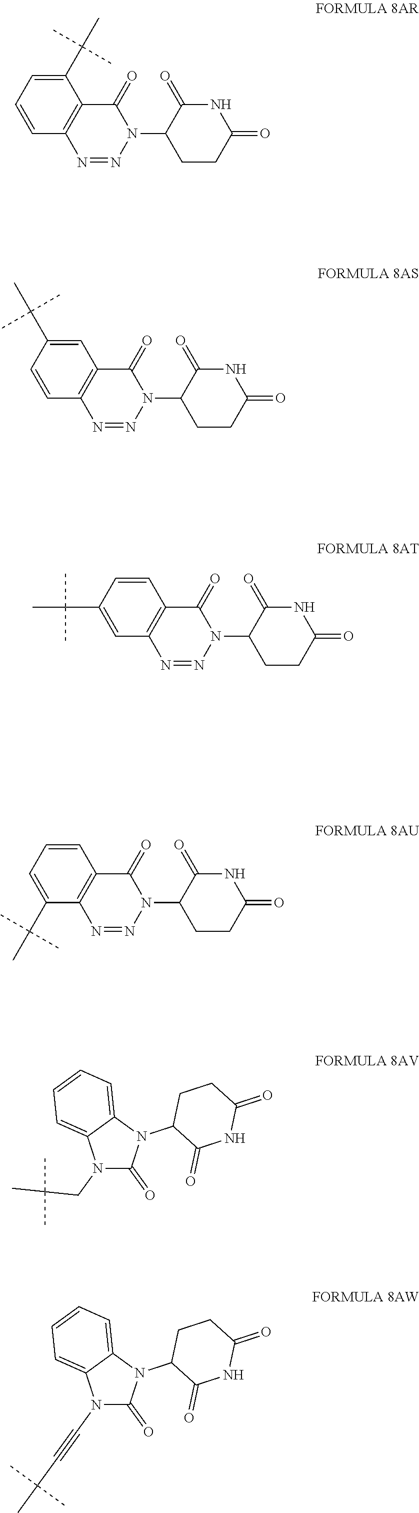

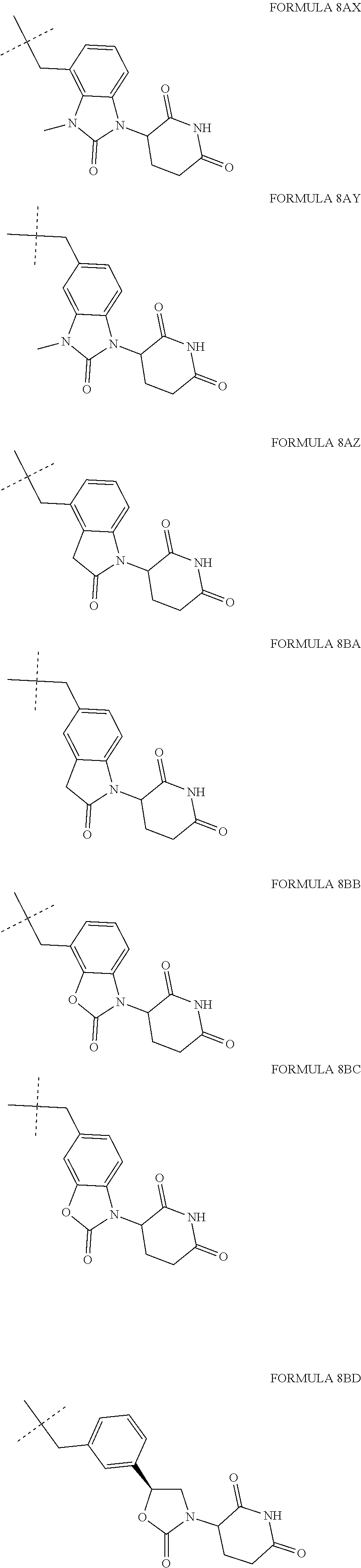

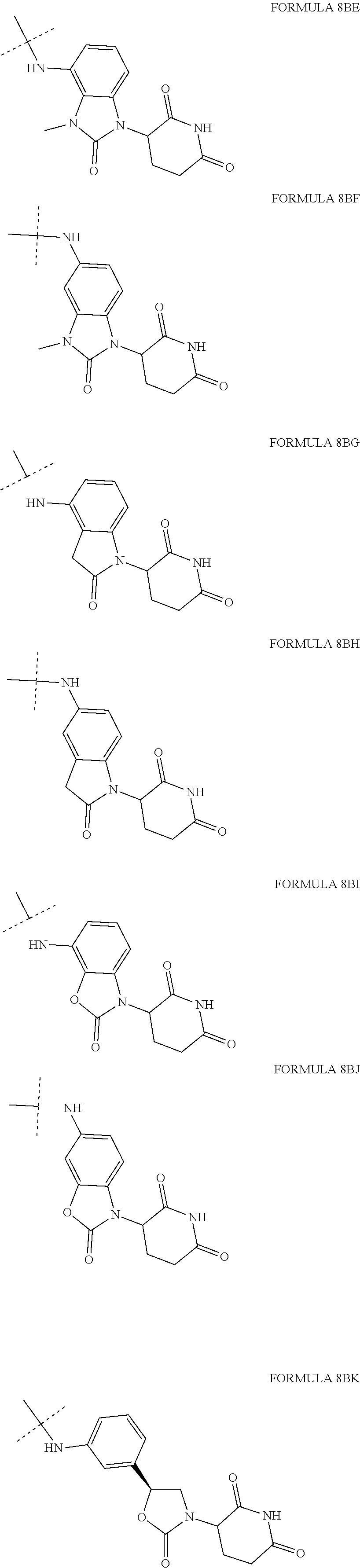

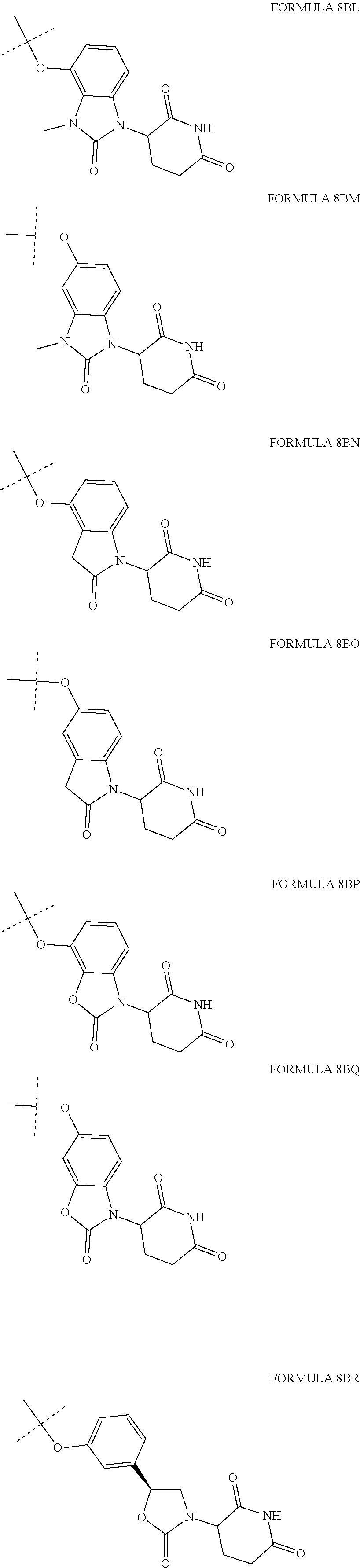

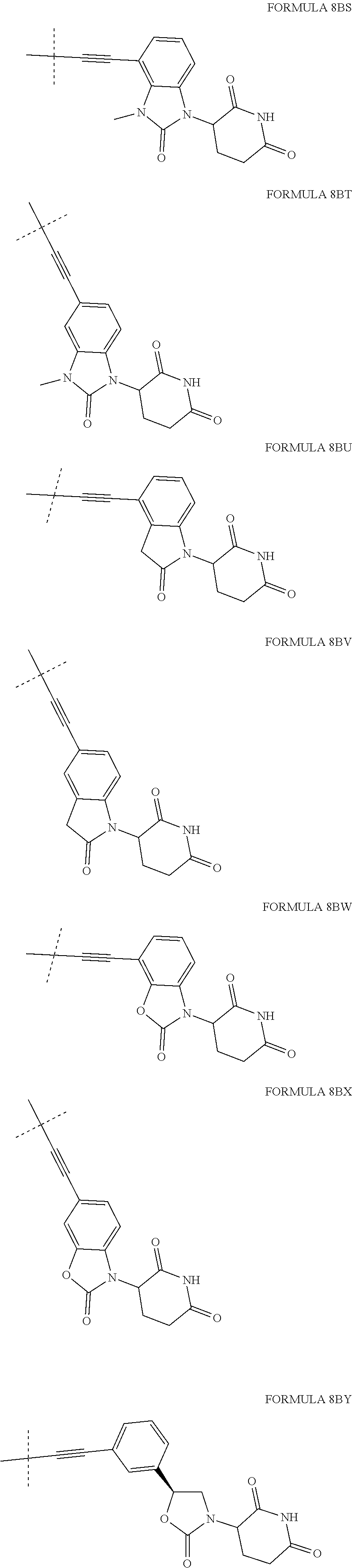

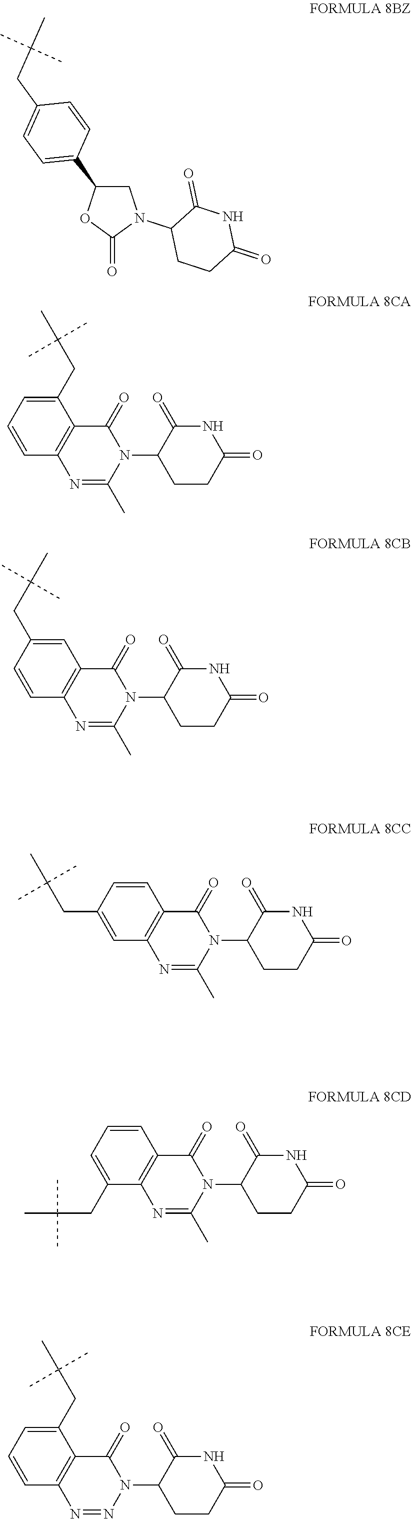

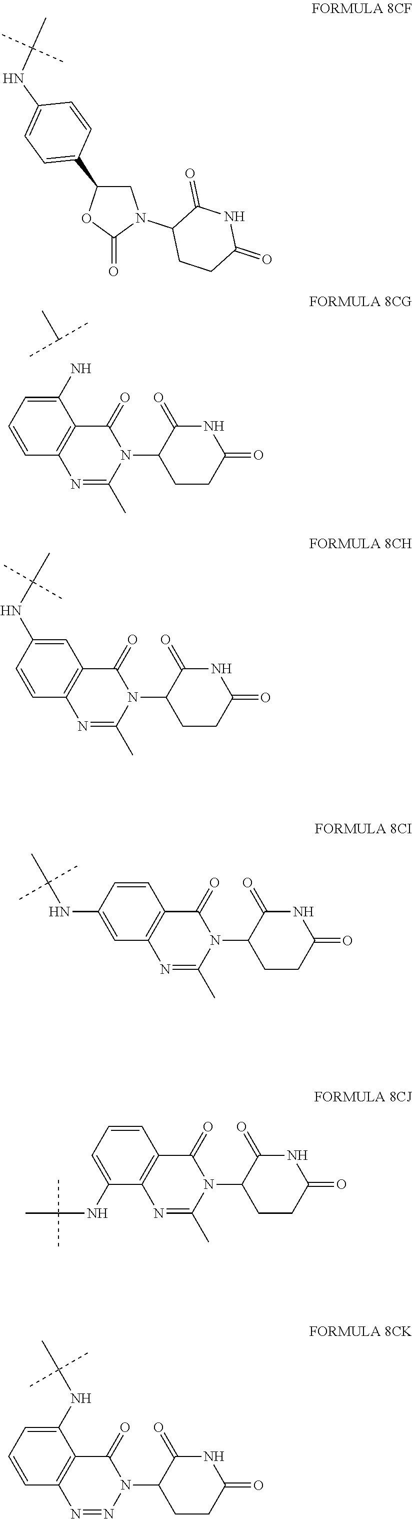

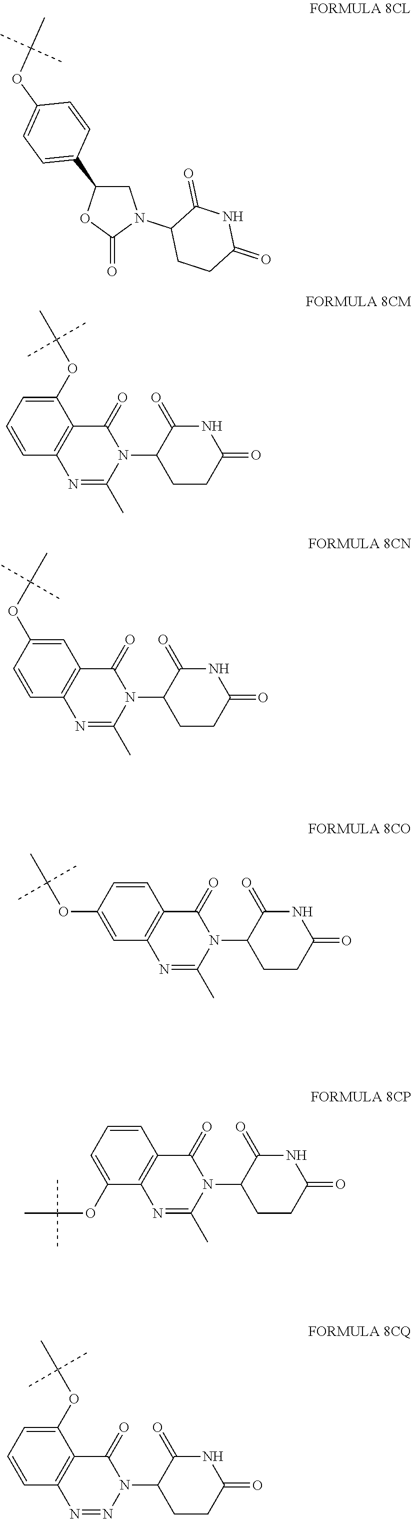

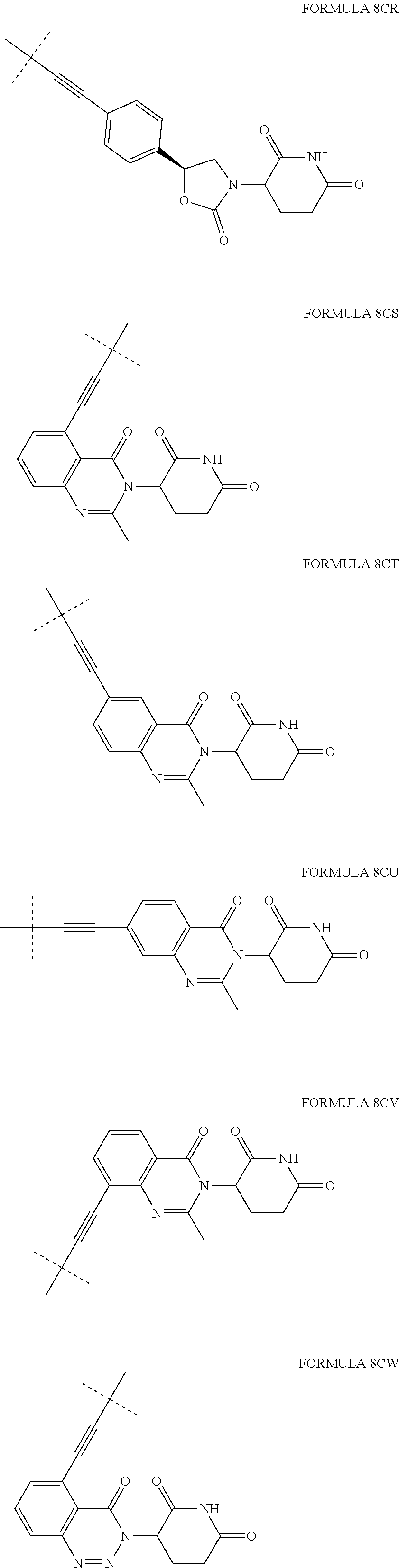

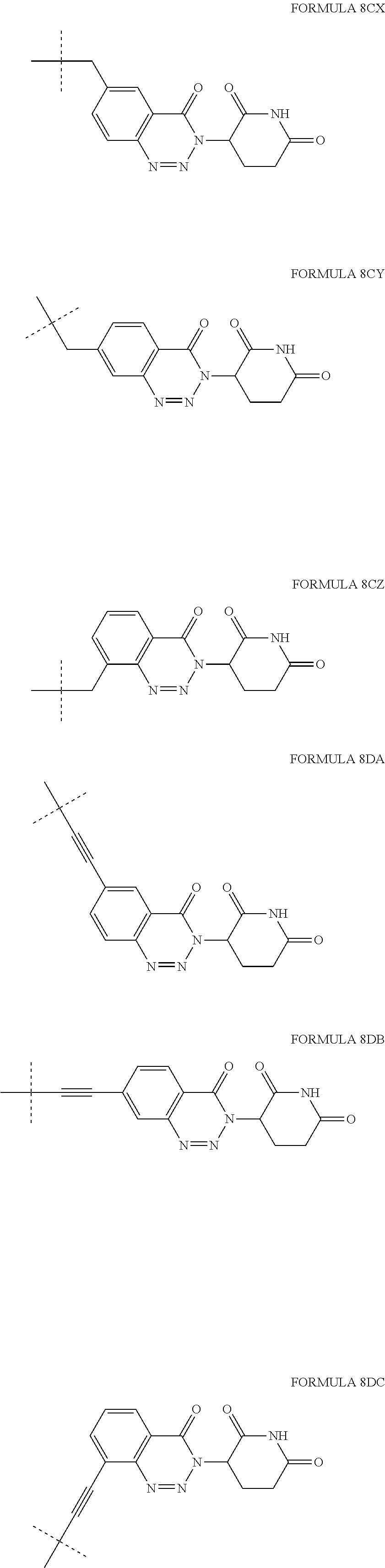

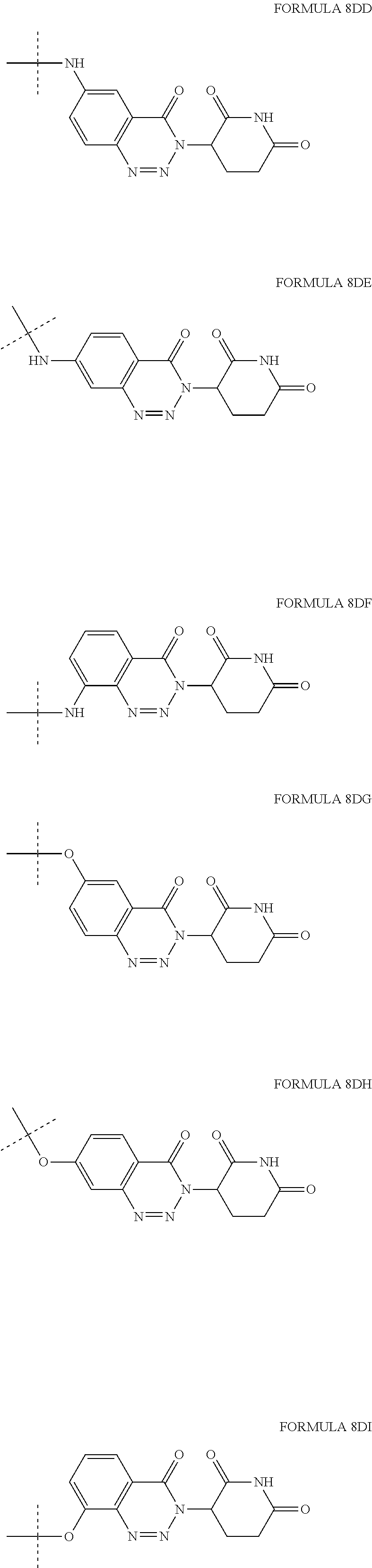

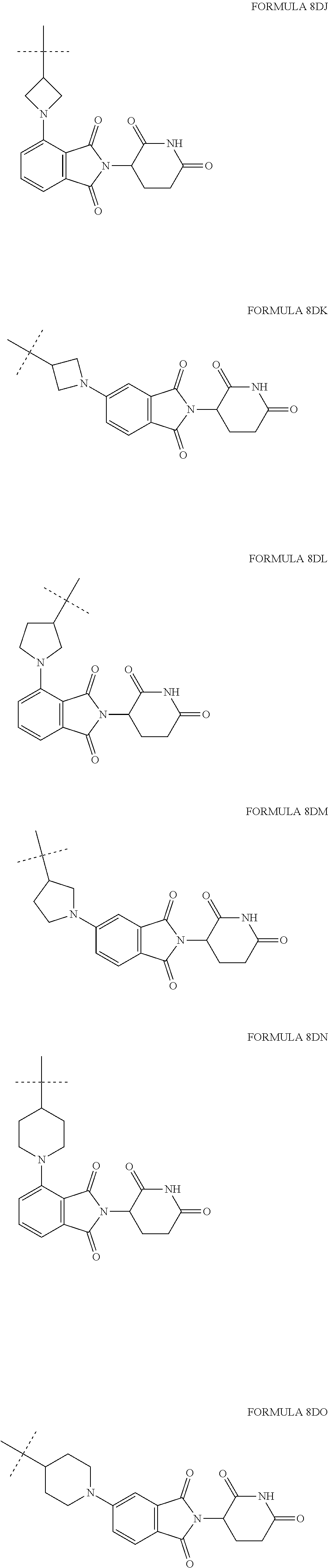

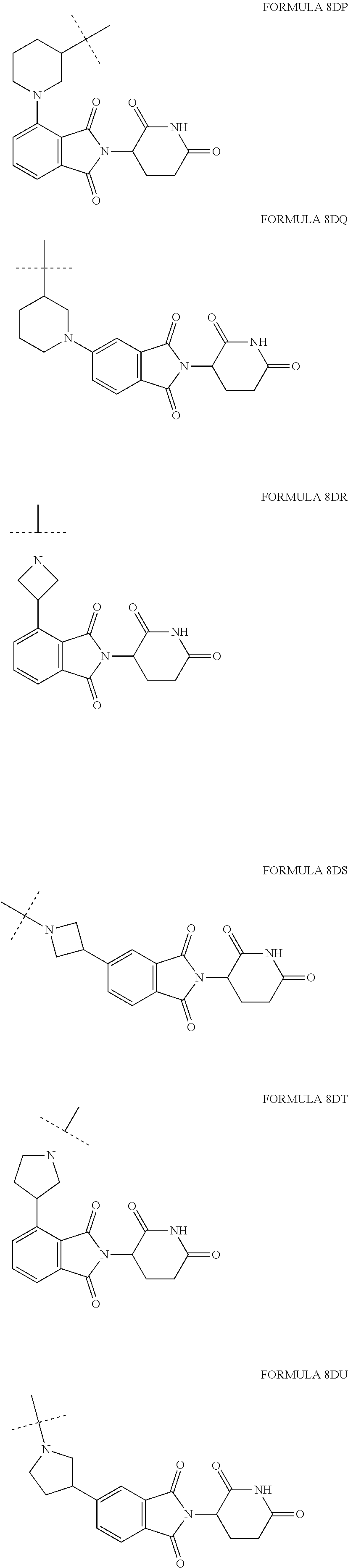

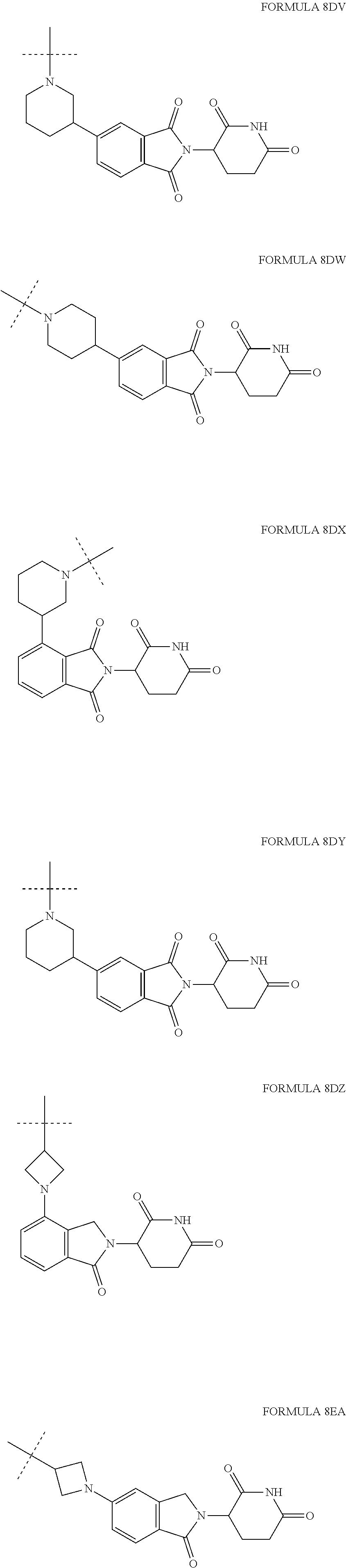

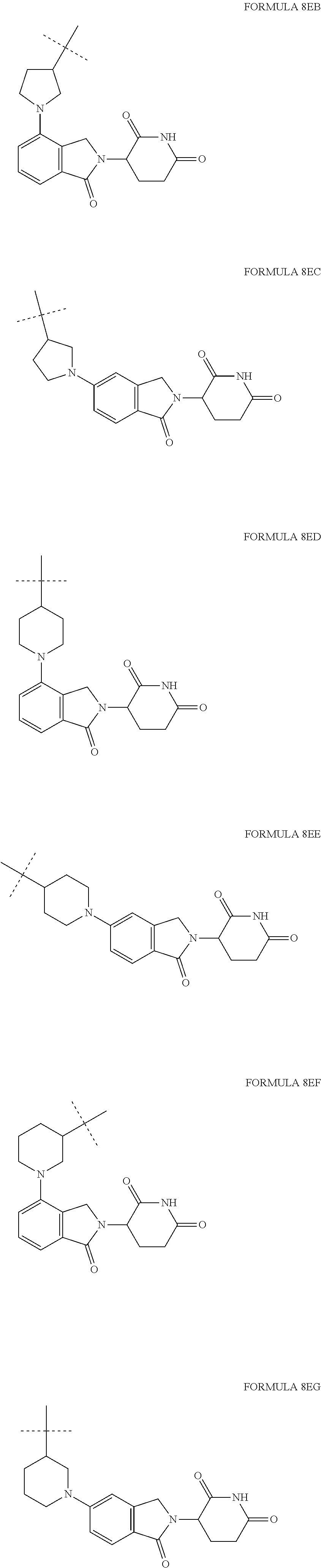

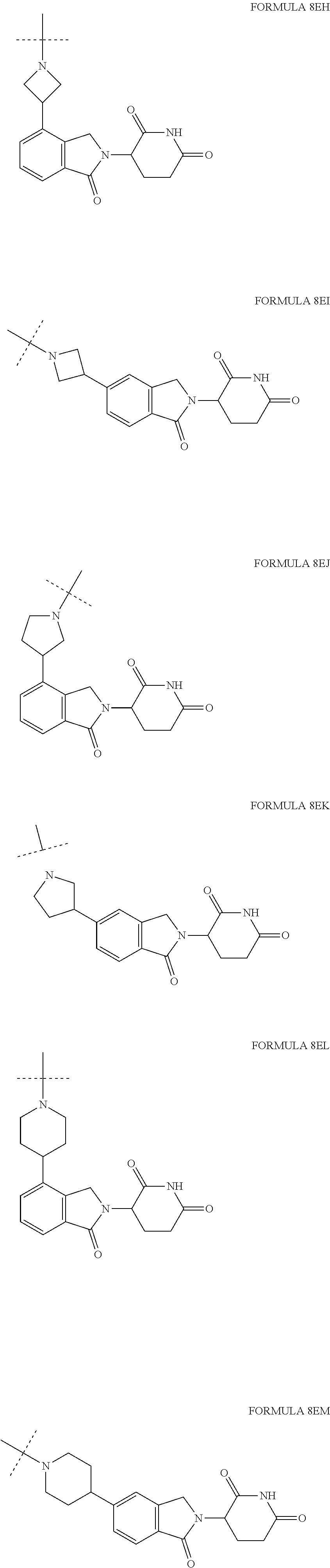

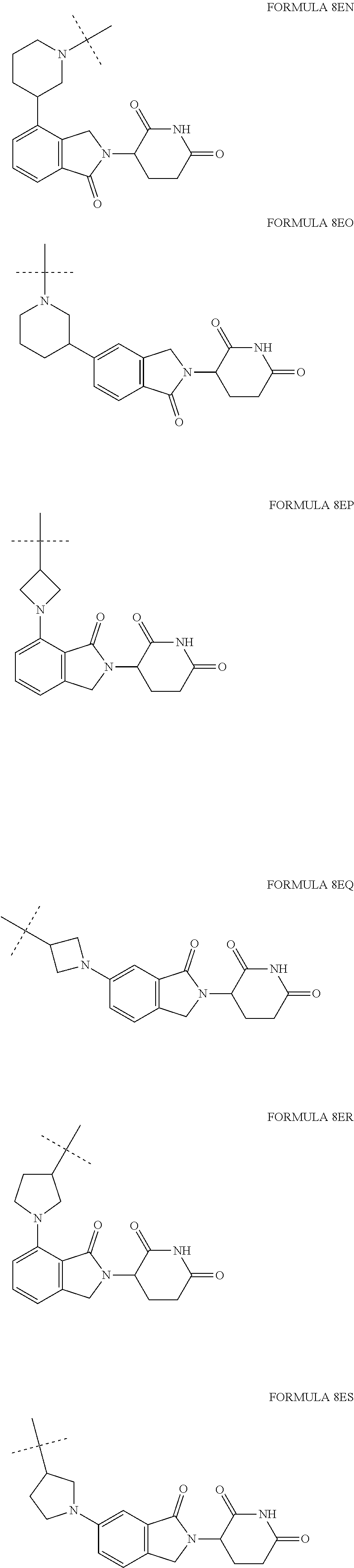

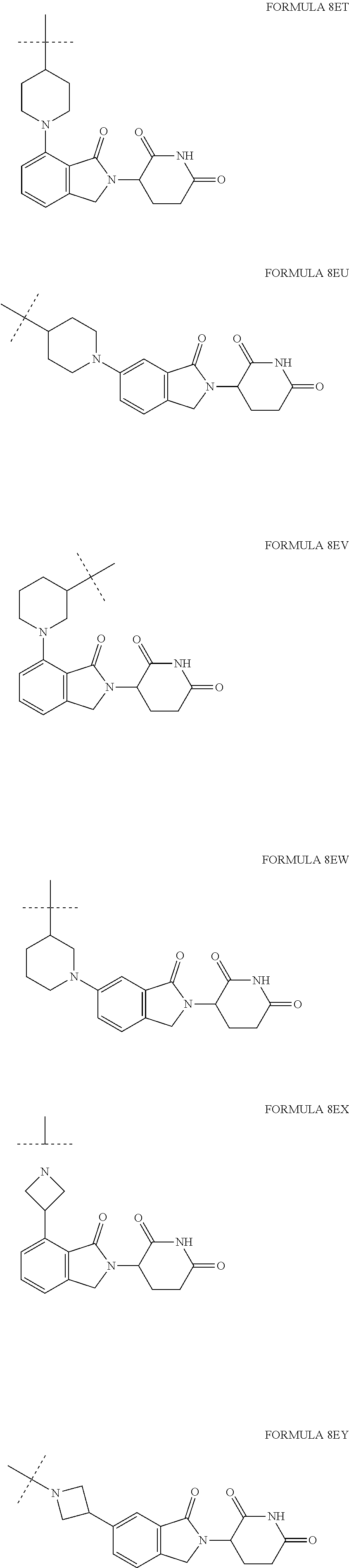

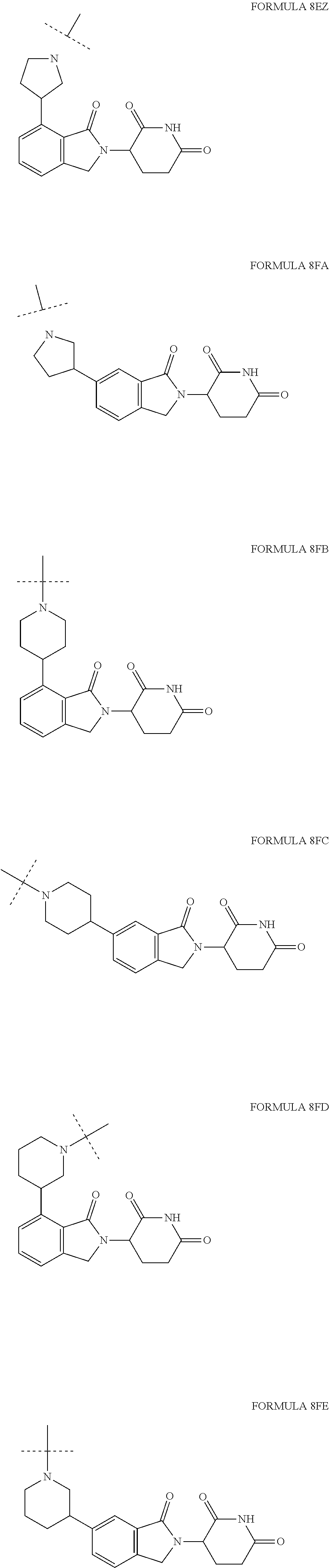

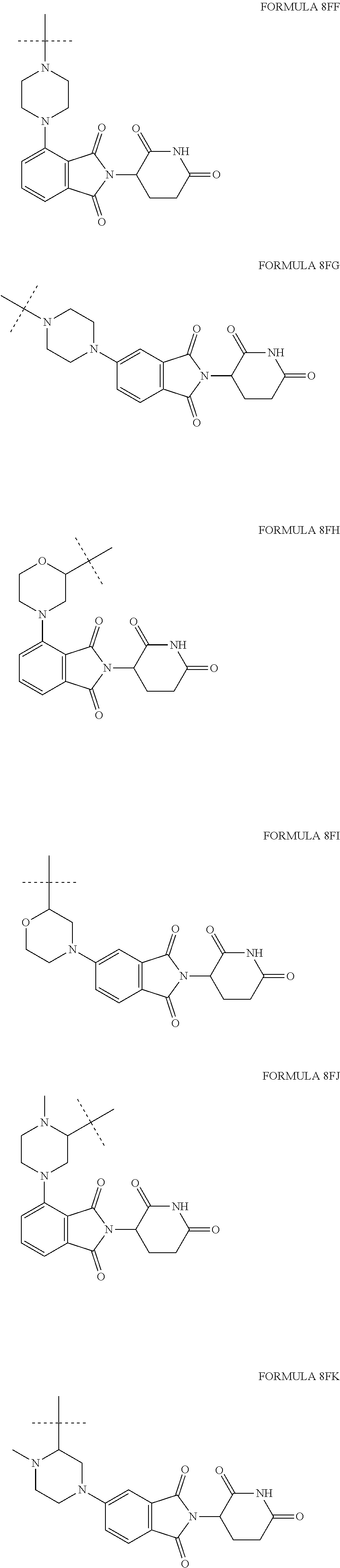

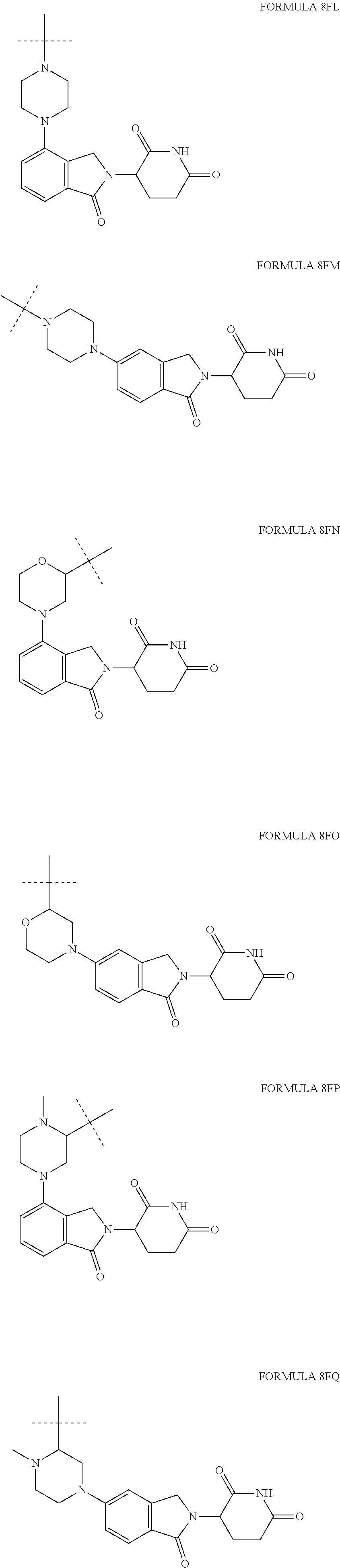

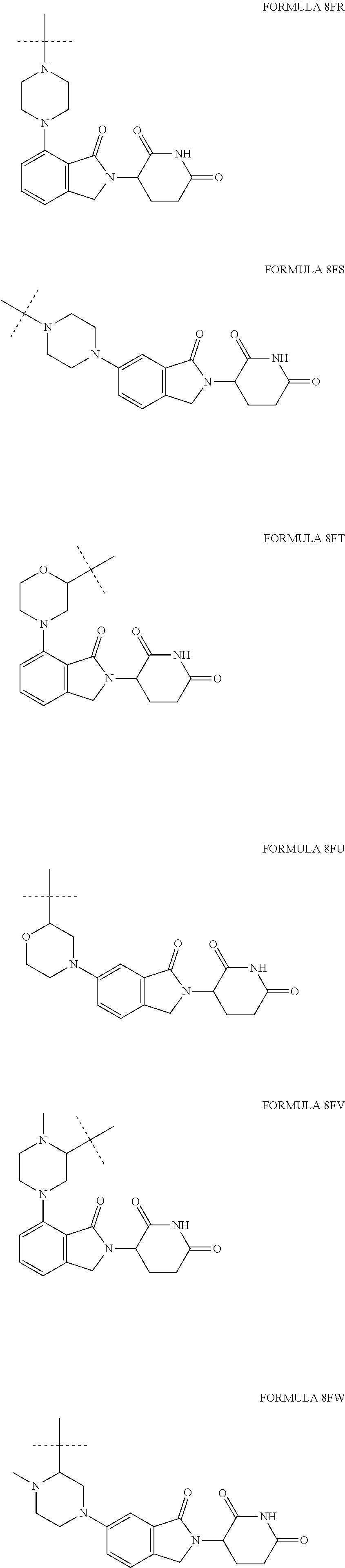

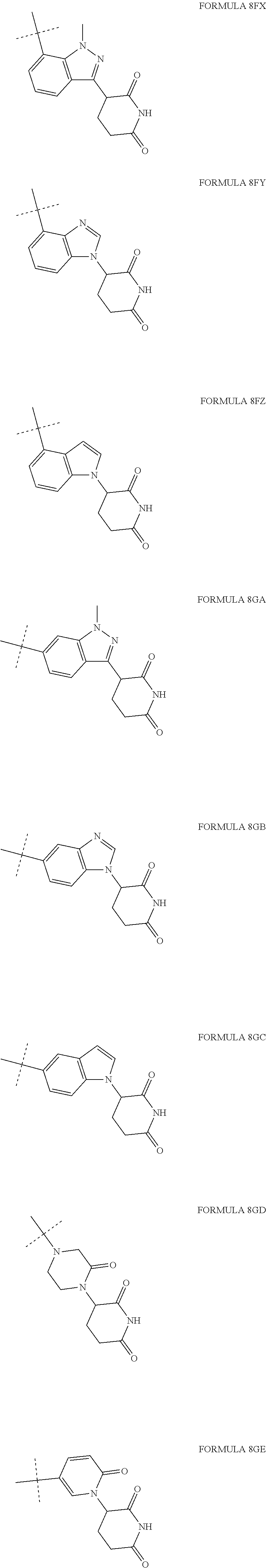

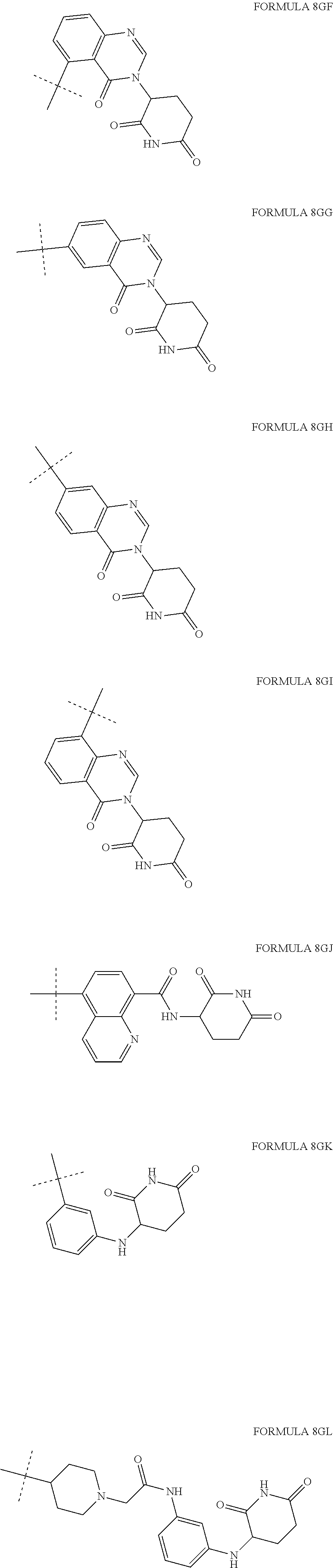

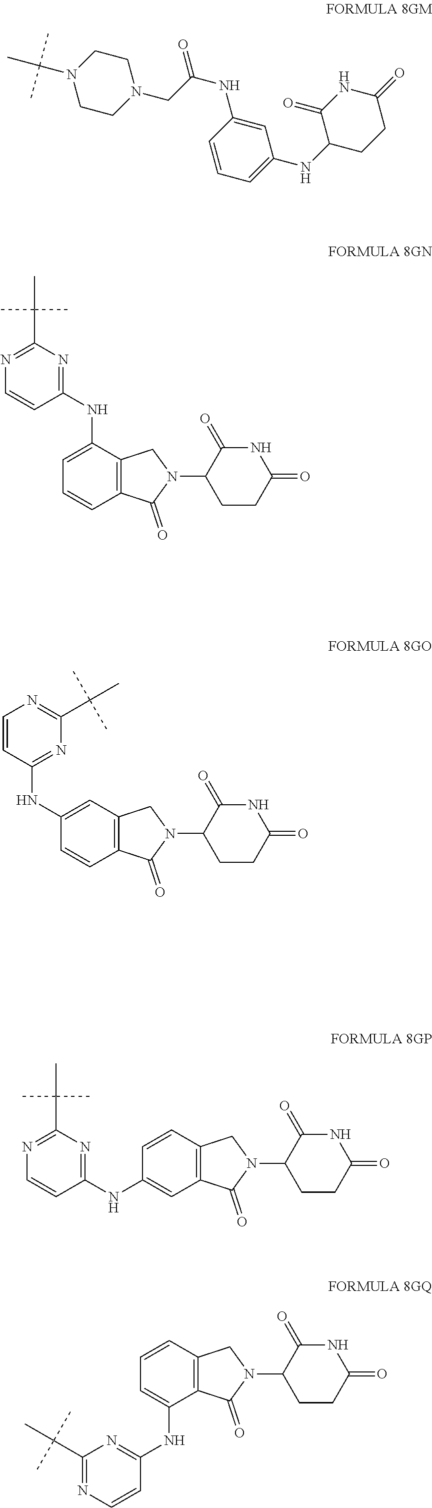

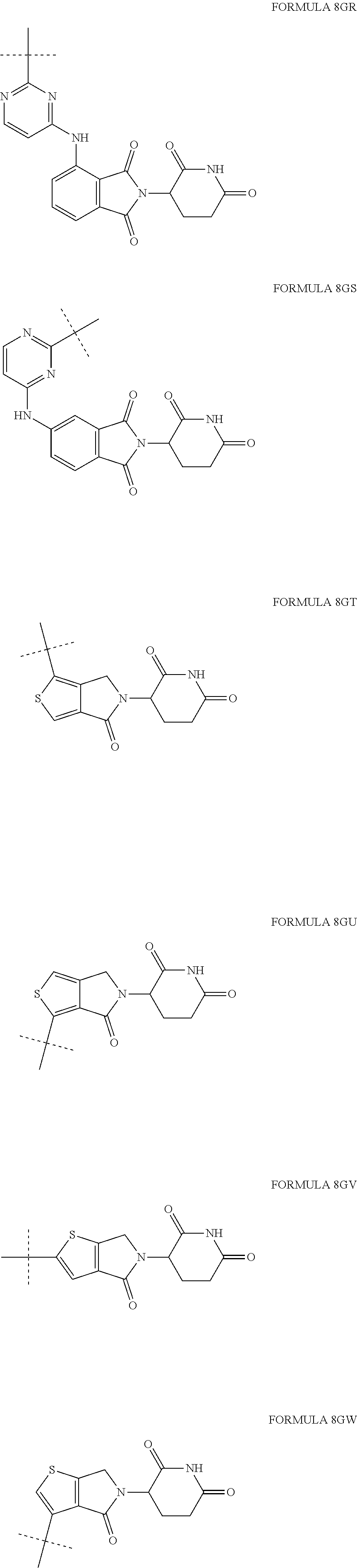

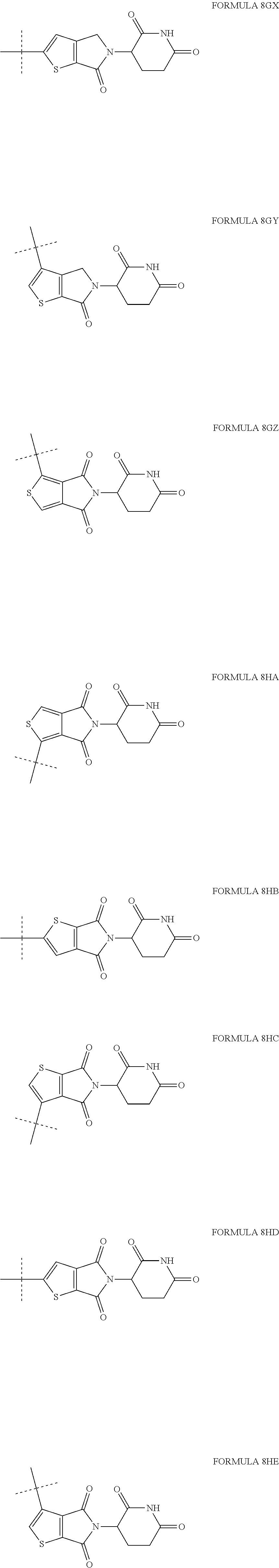

In some embodiments, the linker moeity is of FORMULA 9:

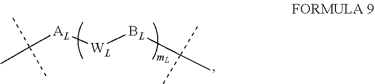

wherein
A_{L}, W_{L} and B_{L}, at each occurrence, are independently selectred from null, or divalent moeity selected from R_{L}^{d}—R_{L}^{e}, R_{L}^{d}COR_{L}^{e}, R_{L}^{d}CO₂R_{L}^{e}, R_{L}^{d}C(O)N(Rₗ¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹R_{L}^{e}, R_{L}^{d}N(R_{L}¹)CORR_{L}^{e}, R_{L}^{d}N(R_{L}¹)CON(R_{L}²)R_{L}^{e}, R_{L}^{d}N(R_{L}¹)C(S)R_{L}^{e}, optionally substitued C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ heteroalkylene, optionally substituted C₁-C₈ hydoxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocycyl, optionally substituted 5-13 membered spiro carbocyclyl, optionally substituted 5-13 membered spiro heterocylyl, optionaly substituted 3-10 membered carbocyclyl, optionally substituted 3-10 membered heterocyclyl, optionally substituted aryl, and optionally substituted heroaryl, wherein
R_{L}^{d} and R_{L}^{e} are independently selected from null, optionally substituted (C₁-C₈ alkylene)-R_{L}^{r}(preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ heteroalkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}^{r} is selected from optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}¹ and R_{L}² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 3-10 membered heterocyclyl, optionally substituted aryl, and optionally substituted heteroaryl;
R_{L}^{d} and R_{L}^{e}, R_{L}¹ and R_{L}², R_{L}^{d} and R_{L}¹, R_{L}^{d} and R_{L}², R_{L}^{e} and R_{L}¹, R_{L}^{e} and R_{L}² together with the atom(s) to which they are connected form a 3-20 membered carbocyclyl or 4-20 membered heterocyclyl ring; and
m_{L} is 1, 2, 3, 4, 5, 6, 7, 8, 9, 10, 11, 12, 13, 14, or 15.

In another embodiment, W_{L} and m_{L} are defined as above; and A_{L} and B_{L}, at each occurrence, are independently selected from null, or divalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, R_{L}^{d}N(R_{L}¹)C(S)R_{L}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ heteroalkylene, optionally substituted C₁-C₈ haloalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl, wherein R_{L}^{d} and R_{L}^{e} are independently selected from null, optionally substituted (C₁-C₈ alkylene)-R_{L}^{r} (preferably, CH₂—R_{L}^{r}, optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl.

In one embodiment, A_{L} is selected from optionally substituted C₁-C₈ alkylene. In another embodiment, A_{L} is methylene. In a further embodiment, A_{L} is ethylene. In yet another embodiment, A_{L} is is n-propylene. In yet a further embodiment, A_{L} is null. In yet a further embodiment, A_{L} is R_{L}^{d}COR_{L}^{e} where R_{L}^{d} is CH₂ and R_{L}^{e} is CH₂. In yet another embodiment, A_{L} is R_{L}^{d}COR_{L}^{e} where R_{L}^{d} is CH₂CH₂ and R_{L}^{e} is CH₂. In yet a further embodiment, A_{L} is R_{L}^{d}COR_{L}^{e} where R_{L}^{d} is CH₂ and R_{L}^{e} is CH₂CH₂. In one embodiment, A_{L} is R_{L}^{d}COR_{L}^{e} where R_{L}^{d} and R_{L}^{e} are both null. In yet another embodiment, A_{L} is n-propylene optionally substutituted with at least one Cl, Br, I, F, OH, NH₂, SH, or OCH₃. In another embodiment, A_{L} is aryl. In a further embodiment, A_{L} is phenyl optionally substituted with at least one Cl, Br, I, F, OH, NH₂, SH, or OCH₃. In yet another embodiment, A_{L} is an optionally substituted heteroaryl group. In a further embodiment, A_{L} is R_{L}^{d}COR_{L}^{e} where R_{L}^{d} is null and R_{L}^{e} is CH₂CH₂CH₂. In yet another embodiment, A_{L} is an optionally substituted heteroaryl group. In a further embodiment, A_{L} is R_{L}^{d}COR_{L}^{e} where R_{L}^{d} is null and R_{L}^{e} is CH₂CH₂CH₂CH₂. In yet another embodiment, A_{L} is an optionally substituted heteroaryl group selected from pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzotbiazolyl, benzisothiazolyl, benzisoxazolyl, benzoxadiazolyl, benzoxazolyl, cinnolinyl, 1H-indazolyl, 2H-indazolyl, indolizinyl, isobenzofuryl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, and thienoimidazolyl.

In one embodiment, B_{L} is selected from optionally substituted C₁-C₈ alkylene. In another embodiment, B_{L} is methylene. In a further embodiment, B_{L} is ethylene. In yet another embodiment, B_{L} is is n-propylene. In yet a further embodiment, B_{L} is null. In yet a further embodiment, B_{L} is R_{L}^{d}COR_{L}^{e} where R_{L}^{d} is CH₂ and R_{L}^{e} is CH₂. In yet another embodiment, B_{L} is R_{L}^{d}COR_{L}^{e} where R_{L}^{d} is CH₂CH₂ and RLi is CH₂. In yet a further embodiment, B_{L} is R_{L}^{d}COR_{L}^{e} where R_{L}^{d} is CH₂ and R_{L}^{e} is CH₂CH₂. In one embodiment, B_{L} is R_{L}^{d}COR_{L}^{e} where R_{L}^{d} and R_{L}^{e} are both null. In yet another embodiment, B_{L} is n-propylene optionally substituted with at least one Cl, Br, I, F, OH, NH₂, SH, or OCH₃. In another embodiment, B_{L} is aryl. In a further embodiment, B_{L} is phenyl optionally substituted with at least one Cl, Br, I, F, OH, NH₂, SH, or OCH₃. In yet another embodiment, B_{L} is an optionally substituted heteroaryl group. In a further embodiment, B_{L} is R_{L}^{d}COR_{L}^{e} where R_{L}^{d} is null and R_{L}^{e} is CH₂CH₂CH₂. In yet another embodiment, B_{L} is an optionally substituted heteroaryl group. In a further embodiment, B_{L} is R_{L}^{d}COR_{L}^{e} where R_{L}^{d} is null and R_{L}^{e} is CH₂CH₂CH₂CH₂. In yet another embodiment, B_{L} is an optionally substituted heteroaryl group selected from pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzotbiazolyl, benzisothiazolyl, benzisoxazolyl, benzoxadiazolyl, benzoxazolyl, cinnolinyl, 1H-indazolyl, 2H-indazolyl, indolizinyl, isobenzofuryl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, and thienoimidazolyl.

In another embodiment R_{L}^{r} is selected from FORMULAE C1, C2, C3, C4 and C5

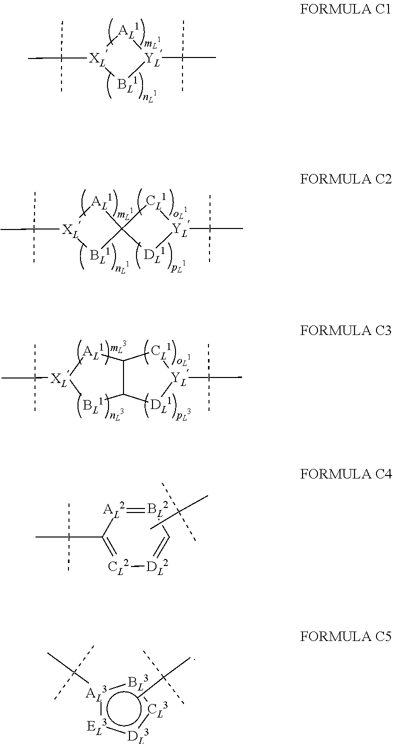

wherein
A_{L}¹, B_{L}¹, C_{L}¹ and D_{L}¹, at each occurrence, are independently selected from null, O, CO, SO, SO₂, NR_{L}^{b}, or CR_{L}^{b}R_{L}^{c};
X_{L}′ and Y_{L}′, at each occurrence, are independently selected from N, or CR_{L}^{b};
A_{L}², B_{L}², C_{L}², D_{L}² and E_{L}², at each occurrence, are independently selected from N, or CR_{L}^{b};
A_{L}³, B_{L}³, C_{L}³, D_{L}³, and E_{L}³, at each occurrence, are independently selected from N, O, S, NR_{L}^{b}, or CR_{L}^{b}.
R_{L}^{b} and R_{L}^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered membered heterocyclyl, optionally substituted aryl, and optionally substituted heteroaryl; and
m_{L}¹, n_{L}¹, o_{L}¹ and p_{L}¹ are independently selected from 0, 1, 2, 3, 4 and 5.

In another embodiment, R_{L}^{r} is selected from Group R_{L}, and Group R_{L} consists of

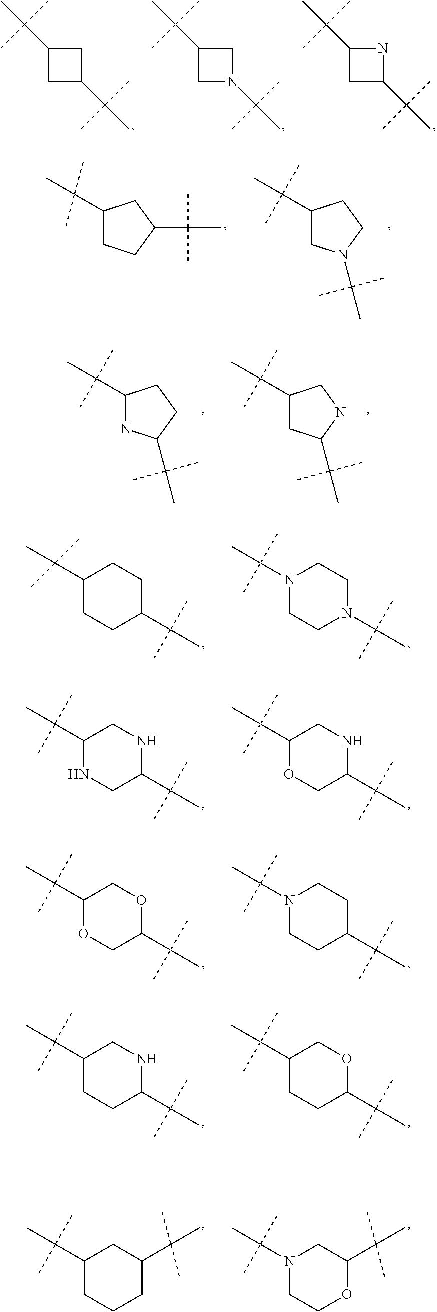

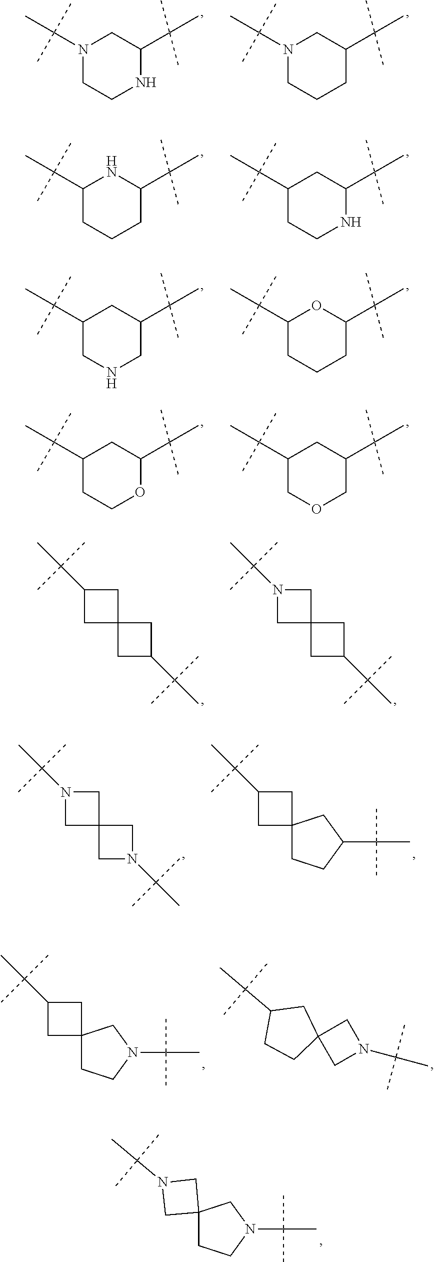

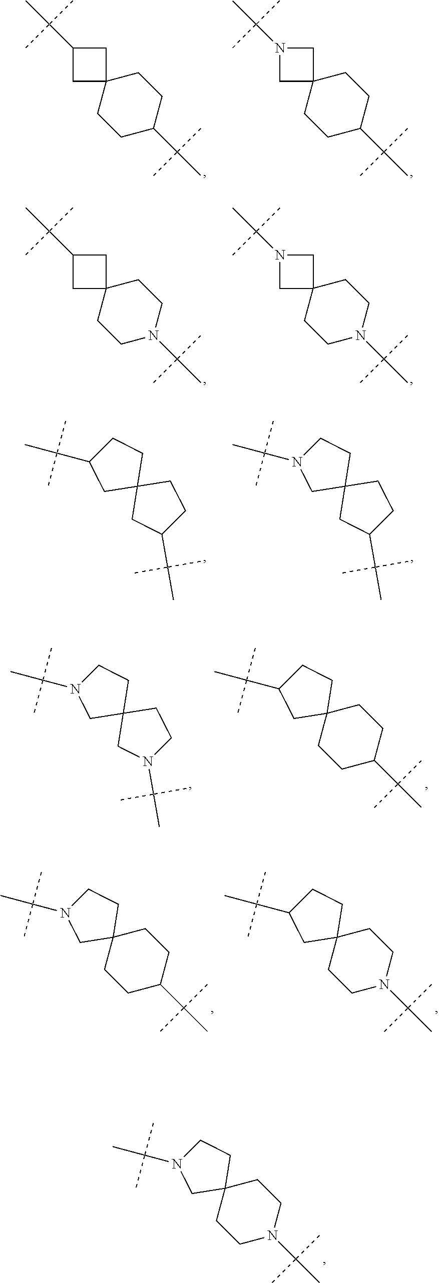

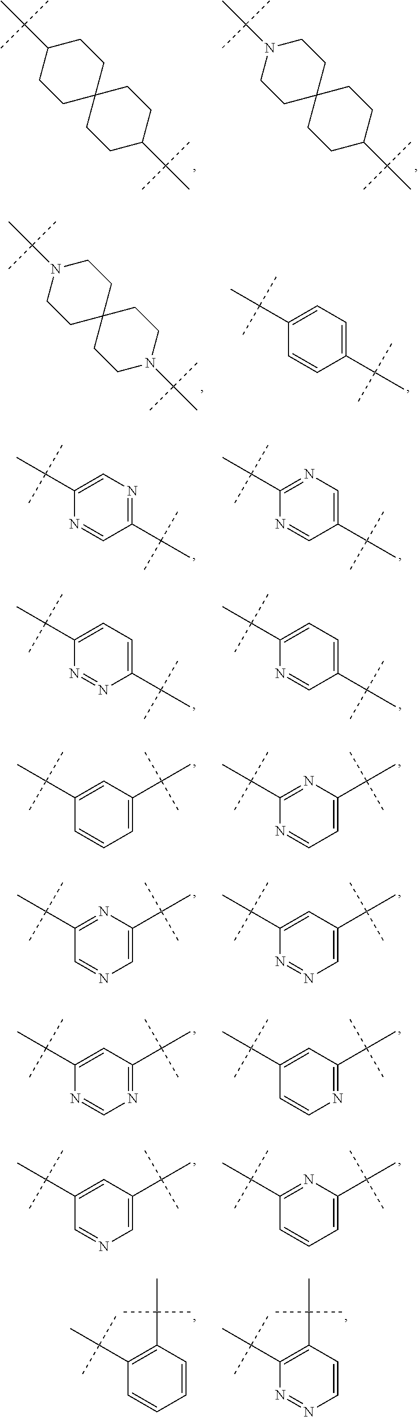

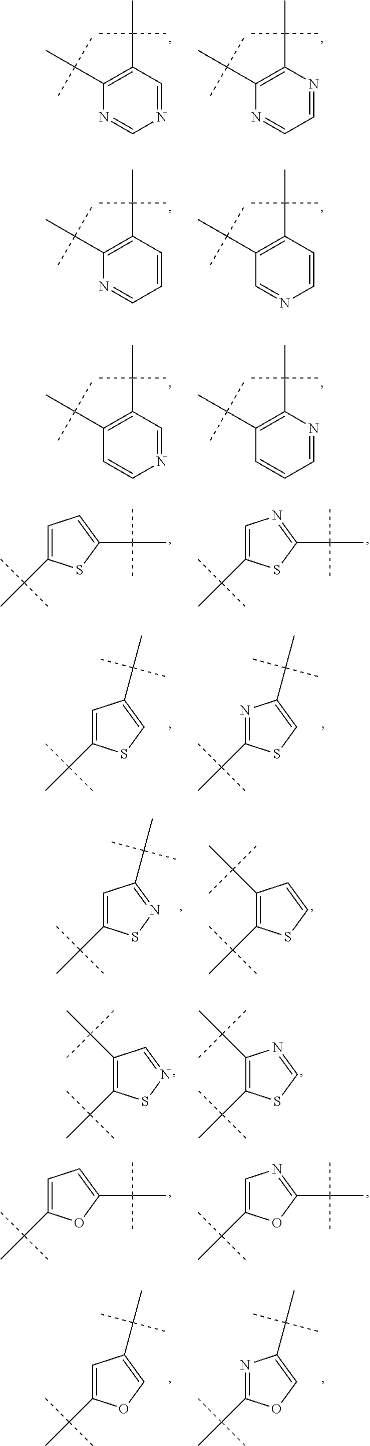

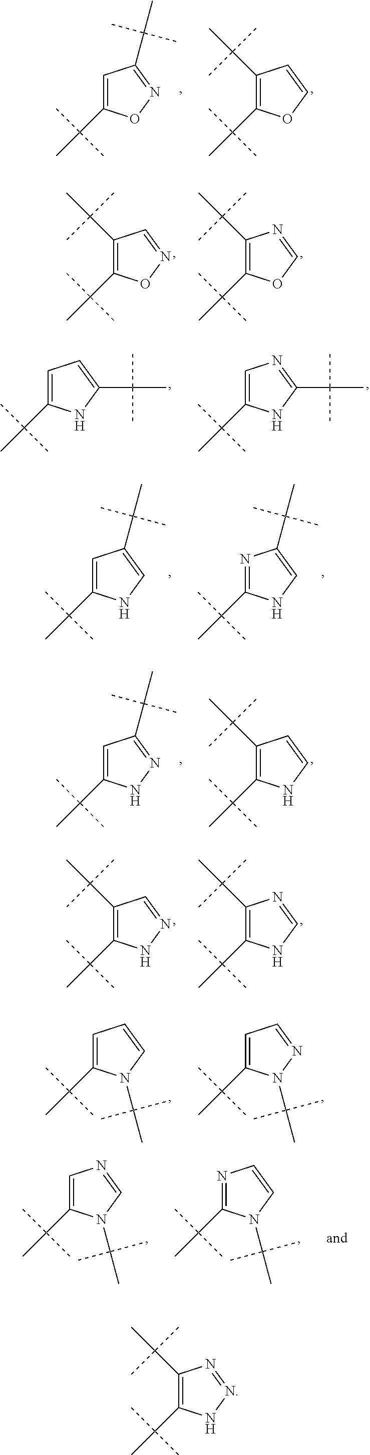

In one embodiment, the linker moiety is of FORMULA 9A:

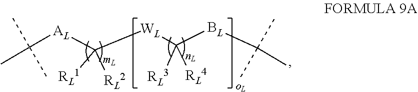

wherein
R_{L}¹, R_{L}², R_{L}³ and R_{L}⁴, at each occurrence, are independently Selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 3-8 membered membered heterocyclyl, optionally substituted aryl, and optionally substituted heteroaryl, or
R_{L}¹ and R_{L}², R_{L}³ and R_{L}⁴ together with the atom(s) to which they are connected form a 3-20 membered carbocyclyl or 4-20 membered heterocyclyl ring;
A_{L}, W_{L} and B_{L}, at each occurrence, are independently selected from null, or divalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}⁵)R_{L}^{e}, R_{L}^{d}C(S)N(R_{L}⁵)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}⁵)R_{L}^{e}, R_{L}^{d}N(R_{L}⁵)R_{L}^{e}, R_{L}^{d}N(R_{L}⁵)COR_{L}^{e}, R_{L}^{d}N(R_{L}⁵)CON(R_{L}⁶)R_{L}^{e}, R_{L}^{d}N(R_{L}⁵)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ heteroalkylene, optionally substituted C₁-C₈ haloalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl, wherein
R_{L}^{d} and R_{L}^{e} are independently selected from null, optionally substituted (C₁-C₈ alkyl)-R_{L}^{r}(preferably, CH₂—R_{L}^{r}), optionally substituted R_{L}^{r}-(C₁-C₈ alkylene), optionally substituted (C₁-C₈ alkylene)-R_{L}^{r}—(C₁-C₈ alkylene), or a moiety comprising of optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ heteroalkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}^{r} is selected from optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 4-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}⁵ and R_{L}⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ heteroalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 3-10 membered heterocyclyl, optionally substituted aryl, and optionally substituted heteroaryl;
R_{L}^{d} and R_{L}^{e}, R_{L}⁵ and R_{L}⁶, R_{L}^{d} and R_{L}, R_{L}^{d} and R_{L}⁶, R_{L}^{e} and R_{L}⁵, R_{L}^{e} and R_{L}⁶ together with the atom(s) to which they are connected form a 3-20 membered carbocyclyl or 4-20 membered heterocyclyl ring;
m_{L} is 1, 2, 3, 4, 5, 6, 7, 8, 9, 10, 11, 12, 13, 14, or 15;
n_{L}, at each occurrence, is 1, 2, 3, 4, 5, 6, 7, 8, 9, 10, 11, 12, 13, 14, or 15; and
o_{L} is 1, 2, 3, 4, 5, 6, 7, 8, 9, 10, 11, 12, 13, 14, or 15.

In another embodiment, A_{L}, W_{L} and B_{L}, at each occurrence, are independently selected from null, or divalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}⁵)R_{L}^{e}, R_{L}^{d}C(S)N(R_{L}⁵)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}⁵)R_{L}^{e}, R_{L}^{d}N(R_{L}⁵)R_{L}^{e}, R_{L}^{d}N(R_{L}⁵)COR_{L}^{e}, R_{L}^{d}N(R_{L}⁵)CON(R_{L}⁶)R_{L}^{e}, R_{L}^{d}N(R_{L}⁵)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ heteroalkylene, optionally substituted C₁-C₈ haloalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl.

In another embodiment, the linker moiety is of FORMULA 9B:

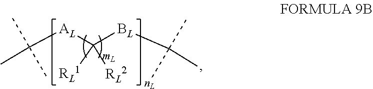

wherein
R_{L}¹ and R_{L}², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ heteroalkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 3-10 membered heterocyclyl, optionally substituted aryl, and optionally substituted heteroaryl, or
R_{L}¹ and R_{L}² together with the atom(s) to which they are connected form a 3-20 membered carbocyclyl or 4-20 membered heterocyclyl ring;
A_{L} and B_{L}, at each occurrence, are independently selected from null, or divalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}³)R_{L}^{e}, R_{L}^{d}C(S)N(R_{L}³)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}³)R_{L}^{e}, R_{L}^{d}N(R_{L}³)R_{L}^{e}, R_{L}^{d}N(R_{L}³)COR_{L}^{e}, R_{L}^{d}N(R_{L}³)CON(R_{L}⁴)R_{L}^{e}, R_{L}^{d}N(R_{L}³)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ heteroalkylene, optionally substituted C₁-C₈ haloalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl, wherein
R_{L}^{d} and R_{L}^{e} are independently selected from null, optionally substituted (C₁-C₈ alkylene)-R_{L}^{r}(preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}^{r} is selected from optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}³ and R_{L}⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ heteroalkyl, optionally substituted C₁-C₈ haloalkyl, optionally substituted C₁-C₈ hydroxyalkyl, optionally substituted C₁-C₈akylamino C₁-C₈alkyl, optionally substituted 3-10 membered carbocyclyl, optionally substituted 3-10 membered heterocyclyl, optionally substituted aryl, and optionally substituted heteroaryl;
R_{L}^{d} and R_{L}^{e}, R_{L}³ and R_{L}⁴, R_{L}^{d} and R_{L}³, R_{L}^{d} and R_{L}⁴, R_{L}^{e} and R_{L}³, R_{L}^{e} and R_{L}⁴ together with the atom(s) to which they are connected form a 3-20 membered carbocyclyl or 4-20 membered heterocyclyl ring;
each m_{L} is 0, 1, 2, 3, 4, 5, 6, 7, 8, 9, 10, 11, 12, 13, 14, or 15; and
n_{L} is 0, 1, 2, 3,4,5,6,7,8, 9, 10, 11, 12, 13, 14, or 15.

In another embodiment, A_{L} and B_{L}, at each occurrence, are independently selected from null, or divalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}¹)R_{L}^{e}, R_{L}^{d}C(S)N(R_{L}³)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}³)R_{L}^{e}, R_{L}^{d}N(R_{L}³)R_{L}^{e}, R_{L}^{d}N(R_{L}³)COR_{L}^{e}, R_{L}^{d}N(R_{L}³)CON(R_{L}⁴)R_{L}^{e}, R_{L}^{d}N(R_{L}³)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ heteroalkylene, optionally substituted C₁-C₈ haloalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl, wherein R_{L}^{d} and R_{L}^{e} are independently selected from null, optionally substituted (C₁-C₈ alkylene)-R_{L}^{r} (preferably, CH₂—R_{L}¹), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ heteroalkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl.

In one embodiment, A_{L} is selected from optionally substituted C₁-C₃ alkylene. In another embodiment, A_{L} is methylene. In a further embodiment, A_{L} is ethylene. In yet another embodiment, A_{L} is is n-propylene. In yet a further embodiment, A_{L} is null. In yet a further embodiment, A_{L} is R_{L}^{d}COR_{L}^{e} where R_{L}^{d} is CH₂ and R_{L}^{e} is CH₂. In yet another embodiment, A_{L} is R_{L}^{d}COR_{L}^{e} where R_{L}^{d} is CH₂CH₂ and R_{L}^{e} is CH₂. In yet a further embodiment, A_{L} is R_{L}^{d}COR_{L}^{e} where R_{L}^{d} is CH₂ and R_{L}^{e} is CH₂CH₂. In one embodiment, A_{L} is R_{L}^{d}COR_{L}^{e} where R_{L}^{d} and R_{L}^{e} are both null. In yet another embodiment, A_{L} is n-propylene optionally substituted with at least one Cl, Br, I, F, OH, NH₂, SH, or OCH₃. In another embodiment, A_{L} is aryl. In a further embodiment, A_{L} is phenyl optionally substituted with at least one Cl, Br, I, F, OH, NH₂, SH, or OCH₃. In yet another embodiment, A_{L} is an optionally substituted heteroaryl group. In a further embodiment, A_{L} is R_{L}^{d}COR_{L}^{e} where R_{L}^{d} is null and R_{L}^{e} is CH₂CH₂CH₂. In yet another embodiment, A_{L} is an optionally substituted heteroaryl group. In a further embodiment, A_{L} is R_{L}^{d}COR_{L}^{e} where R_{L}^{d} is null and R_{L}^{e} is CH₂CH₂CH₂CH₂. In yet another embodiment, A_{L} is an optionally substituted heteroaryl group selected from pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzotbiazolyl, benzisothiazolyl, benzisoxazolyl, benzoxadiazolyl, benzoxazolyl, cinnolinyl, 1H-indazolyl, 2H-indazolyl, indolizinyl, isobenzofuryl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, and thienoimidazolyl.

In one embodiment, B_{L} is selected from optionally substituted C₁-C₈ alkylene. In another embodiment, B_{L} is methylene. In a further embodiment, B_{L} is ethylene. In yet another embodiment, B_{L} is is n-propylene. In yet a further embodiment, B_{L} is null. In yet a further embodiment, B_{L} is R_{L}^{d}COR_{L}^{e} where R_{L}^{d} is CH₂ and R_{L}^{e} is CH₂. In yet another embodiment, B_{L} is R_{L}^{d}COR_{L}^{e} where R_{L}^{d} is CH₂CH₂ and R_{L}^{e} is CH₂. In yet a further embodiment, B_{L} is R_{L}^{d}COR_{L}C where R_{L}^{d} is CH and R_{L}^{e} is CH₂CH₂. In one embodiment, B_{L} is R_{L}^{d}COR_{L}C where R_{L}^{d} and R_{L}^{e} are both null. In yet another embodiment, B_{L} is n-propylene optionally substutituted with at least one Cl, Br, I, F, OH, NH₂, SH, or OCH₃. In another embodiment, B_{L} is aryl. In a further embodiment, B_{L} is phenyl optionally substituted with at least one Cl, Br, I, F, OH, NH₂, SH, or OCH₃. In yet another embodiment, B_{L} is an optionally substituted heteroaryl group. In a further embodiment, B_{L} is R_{L}^{d}COR_{L}^{e} where R_{L}^{d} is null and R_{L}^{e} is CH₂CH₂CH₂. In yet another embodiment, B_{L} is an optionally substituted heteroaryl group. In a further embodiment, B_{L} is R_{L}^{d}COR_{L}^{e} where R_{L}^{d} is null and R_{L}^{e} is CH₂CH₂CH₂CH₂. In yet another embodiment, B_{L} is an optionally substituted heteroaryl group selected from pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzotbiazolyl, benzisothiazolyl, benzisoxazolyl, benzoxadiazolyl, benzoxazolyl, cinnolinyl, 1H-indazolyl, 2H-indazolyl, indolizinyl, isobenzofuryl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, and thienoimidazolyl.

In another embodiment, R_{L}^{r} is selected from Group R_{L}, and Group R_{L} is defined as in FORMULA 9.

In another embodiment, the linker moiety is of FORMULA 9C:

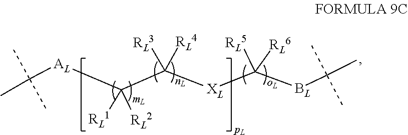

wherein
X_{L} is selected from O and NR_{L}⁷,
R_{L}¹, R_{L}², R_{L}³, R_{L}⁴, R_{L}⁵, and R_{L}⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ heteroalkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered carbocyclyl, optionally substituted 3-8 membered cycloalkoxy, optionally substituted 3-10 membered heterocyclyl, optionally substituted aryl, and optionally substituted heteroaryl;
A_{L} and B_{L} are independently selected from null, or divalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}⁸)R_{L}^{e}, R_{L}^{d}C(S)N(R_{L}⁸)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}⁸)R_{L}^{e}, R_{L}^{d}N(R_{L}⁸)R_{L}^{e}, R_{L}^{d}N(R_{L}⁸)COR_{L}^{e}, R_{L}^{d}N(R_{L}⁸)CON(R_{L}⁹)R_{L}^{e}, R_{L}^{d}N(R_{L}⁸)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ heteroalkylene, optionally substituted C₁-C₈ haloalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl, wherein R_{L}^{d} and R_{L}^{e} are independently selected from null, optionally substituted (C₁-C₈ alkylene)-R_{L}^{r} (preferably, CH₂—R_{L}^{r}), optionally substituted R_{L}^{r}-(C₁-C₈ alkylene), optionally substituted (C₁-C₈ alkylene)-R_{L}^{r}-(C₁-C₈ alkylene), or a moiety comprising of optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ heteroalkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}^{r} is selected from optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}⁷, R_{L}⁸ and R_{L}⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ heteroalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 3-10 membered heterocyclyl, optionally substituted aryl, and optionally substituted heteroaryl;
R_{L}^{d} and R_{L}^{e}, R_{L}⁸ and R_{L}⁹, R_{L}^{d} and R_{L}S, R_{L}^{d} and R_{L}⁹, R_{L}^{e} and R_{L}⁸, R_{L}^{e} and R_{L}⁹ together with the atom(s) to which they are connected form a 3-20 membered carbocyclyl or 4-20 membered heterocyclyl ring;
m_{L}, at each occurrence, is 0, 1, 2, 3, 4, 5, 6, 7, 8, 9, 10, 11, 12, 13, 14, or 15;
n_{L}, at each occurrence, is 0, 1, 2, 3, 4, 5, 6, 7, 8, 9, 10, 11, 12, 13, 14, or 15;
o_{L} is 0, 1, 2, 3, 4, 5, 6, 7, 8, 9, 10, 11, 12, 13, 14, or 15; and
p_{L} is 0, 1, 2, 3,4,5,6,7,8, 9, 10, 11, 12, 13, 14, or 15.

In some embodiments, the length of the linker is 0 chain atoms. In some embodiments, the length of the linker is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 chain atoms, or a range of chain atoms defined by any two of the aforementioned numbers of chain atoms. In another refinement, the length of the linker is 0 to 40 chain atoms. In another refinement, the length of the linker is 1 to 20 chain atoms. In another refinement, the length of the linker is 2 to 12 chain atoms. In some embodiments, the linker is -(3-10 membered carbocyclyl)-(C₁-C₈ alkylene)- or -(3-10 membered heterocyclyl)-(C₁-C₈ alkylene)-.

In another embodiment, R is selected from FORMULA C1a, C2a, C3a, C4a, C5a, C1, C2, C3, C4, and C5 as defined above.

In another embodiment, R^{r} is selected from Group R.

In some embodiments, the compound is selected from in Table 1, Table 2, or Table 3, wherein the compound is not CC-90009 and giltertinib, or a pharmaceutically acceptable salt or solvate thereof. In some embodiments, the compound is GS-025, GS-031, GS-034, GS-035, GS-036, GS-043, GS-044, GS-045, GS-048, GS-049, GS-050, GS-051, GS-052, GS-053, GS-054, GS-060, GS-066, GS-068, GS-069, GS-070, GS-076, GS-077, GS-078, GS-087, GS-090, GS-093, GS-096, GS-097, GS-098, GS-101, GS-102, GS-105, GS-110, GS-113, GS-115, GS-116, GS-118, GS-120, GS-122, GS-123, GS-132, GS-134, GS-145, GS-150, GS-151, GS-156, GS-163, GS-166, GS-167, GS-171, GS-173, GS-174, GS-175, GS-181, GS-182, GS-188, GS-191, GS-194, GS-195, GS-196, GS-198, GS-199, GS-202, GS-204, GS-205, GS-207, GS-210, GS-211, GS-213, GS-215, GS-217, GS-221, GS-223, GS-224, GS-227, GS-228, GS-229, GS-230, GS-231, GS-240, GS-243, GS-247, GS-253, GS-254, GS-255, GS-256, GS-257, GS-259, GS-261, GS-262, GS-264, GS-266, GS-267, GS-272, GS-276, GS-277, GS-278, GS-280, GS-281, GS-282, GS-284, GS-285, GS-286, GS-287, GS-289, GS-299, GS-305, GS-308, GS-309, GS-311, GS-314, GS-315, GS-323, GS-339, GS-341, GS-348, GS-350, GS-351, GS-362, GS-366, GS-368, GS-370, GS-372, GS-377, GS-378, GS-386, GS-387, GS-396, GS-397, GS-399, GS-401, GS-404, GS-405, GS-406, GS-409, GS-410, GS-411, GS-418, GS-419, GS-421, GS-422, GS-426, GS-427, GS-428, GS-429, GS-431, GS-434, GS-442, GS-444, GS-447, GS-458, GS-463, GS-464, GS-467, GS-470, GS-476, GS-477, GS-478, GS-479, GS-480, GS-481, GS-482, GS-483, GS-486, GS-487, GS-488, GS-489, GS-490, GS-491, GS-493, GS-494, GS-495, GS-497, GS-500, GS-501, GS-503, GS-505, GS-509, GS-510, GS-511, GS-512, GS-513, GS-514, GS-518, GS-520, GS-522, GS-525, GS-526, GS-528, GS-529, GS-530, GS-531, GS-532, GS-534, GS-535, GS-541, GS-542, GS-543, GS-544, GS-548, GS-549, GS-552, GS-555, GS-557, GS-559, GS-562, GS-564, GS-567, GS-568, GS-569, GS-570, GS-571, GS-572, GS-573, GS-574, GS-575, GS-576, GS-579, GS-580, GS-581, GS-583, GS-584, GS-585, GS-586, GS-587, GS-589, GS-592, GS-593, GS-594, GS-598, GS-602, GS-603, GS-604, GS-609, GS-610, GS-611, GS-612, GS-613, GS-614, GS-615, GS-616, GS-618, GS-620, GS-621, GS-622, GS-623, GS-625, GS-630, GS-633, GS-634, GS-635, or GS-637; or a pharmaceutically acceptable salt or solvate thereof. In some embodiments, the compound is GS-060, GS-066, GS-105, GS-171, GS-173, GS-181, GS-182, GS-227, GS-243, GS-255, GS-261, GS-262, GS-264, GS-272, GS-286, GS-287, GS-305, GS-309, GS-314, GS-315, GS-339, GS-348, GS-418, GS-442, GS-444, GS-490, GS-510, GS-552, GS-568, GS-570, GS-579, GS-589, GS-613, or GS-614; or a pharmaceutically acceptable salt or solvate thereof. In some embodiments, the compound is GS-171, GS-243, GS-255, GS-264, GS-286, GS-305, GS-442, GS-444, GS-568, GS-579, GS-589, GS-613, or GS-614; or a pharmaceutically acceptable salt or solvate thereof.

In some embodiments, the compound is selected from the group consisting of GS-001 to GS064, or a pharmaceutically acceptable salt or analog thereof. In some embodiments, the compound is selected from the group consisting of GS-002, GS-004, GS-005, GS-006, GS-007, and a pharmaceutically acceptable salt or analog thereof.

In one embodiment, the compound is 5-((6-(4-(4-(8-bromoquinoxalin-2-yl)-1H-pyrazol-1-yl)piperidin-1-yl)-6-oxohexyl)amino)-2-(2,6-dioxopiperidin-3-yl)isoindoline-1,3-dione (GS-002). In some embodiments, the compound comprises GS-002 or a pharmaceutically acceptable salt thereof. In one embodiment, the compound is 2-(3-(4-(4-(8-bromoquinoxalin-2-yl)-1H-pyrazol-1-yl)piperidin-1-yl)phenyl)-N-((2-(2,6-dioxopiperidin-3-yl)-1-oxoisoindolin-5-yl)methyl)-2,2-difluoroacetamide (GS-004). In some embodiments, the compound comprises GS-004 or a pharmaceutically acceptable salt thereof.

In one embodiment, the compound is 2-(2,6-dioxopiperidin-3-yl)-5-((6-oxo-6-(4-(4-(quinolin-2-yl)-1H-pyrazol-1-yl)piperidin-1-yl)hexyl)amino)isoindoline-1,3-dione (GS-006). In some embodiments, the compound comprises GS-006 or a pharmaceutically acceptable salt thereof.

In one embodiment, the compound is 5-(4-(4-(8-bromoquinoxalin-2-yl)-1H-pyrazol-1-yl)piperidin-1-yl)-N-((2-(2,6-dioxopiperidin-3-yl)-1-oxoisoindolin-5-yl)methyl)-2,2-difluoro-5-oxopentanamide (GS-007). In some embodiments, the compound comprises GS-007 or a pharmaceutically acceptable salt thereof.

According to one aspect of the present disclosure, a composition disclosed herein comprises the compound or a pharmaceutically acceptable salt or analog thereof.

In one embodiment, the compound is 2-(2,6-dioxopiperidin-3-yl)-5-((6-oxo-6-(4-(4-(quinoxalin-2-yl)-1H-pyrazol-1-yl)piperidin-1-yl)hexyl)amino)isoindoline-1,3-dione (GS-005). In some embodiments, the compound comprises GS-005 or a pharmaceutically acceptable salt thereof.

Disclosed herein, are GSPT1 degraders. In some embodiments, the compound degrades GSPT1. In some embodiments, the compound reduces cellular GSPT1 protein levels. In some embodiments, the compound binds cereblon. In some embodiments, the compound modulates cereblon. In some embodiments, the compound degrades GSPT1 as a downstream effect of cereblon binding and modulation. Some examples of GSTP1 degraders are shown in Table 1, Table 2, and Table 3.

In some embodiments, the compound inhibits growth of a cell with an IC₅₀ value below 50 nM. In some embodiments, the compound inhibits growth of a cell with an IC₅₀ value below 25 nM. In some embodiments, the compound inhibits growth of a cell with an IC₅₀ value below 10 nM. In some embodiments, the compound inhibits growth of a cell with an IC₅₀ value below 8 nM. In some embodiments, the compound inhibits growth of a cell with an IC₅₀ value below 2 nM. In some embodiments, the cell comprises a MV4;11, or MOLM-13 cell. In some embodiments the cell is MV4;11 cell In some embodiments, the cell is MOLM-13 cell.

In some embodiments, the compound is selected from Compound GS-001 to GS-639, or a pharmaceutically acceptable salt, hydrate, solvate, prodrug, stereoisomer, or tautomer thereof. In some embodiments, the compound is selected from Compound GS-001 to GS-064, or a pharmaceutically acceptable salt, hydrate, solvate, prodrug, stereoisomer, or tautomer thereof. In some embodiments, the compound is selected from Compound GS-064 to GS-639, or a pharmaceutically acceptable salt, hydrate, solvate, prodrug, stereoisomer, or tautomer thereof. In some embodiments, the compound is selected from Compound GS-001 to GS-639, or a pharmaceutically acceptable salt thereof. In some embodiments, the compound is selected from Compound GS-001 to GS-639.

In some embodiments, the compound comprises one of the following, or a pharmaceutically acceptable salt thereof: GS-025, GS-031, GS-034, GS-035, GS-036, GS-043, GS-044, GS-045, GS-048, GS-049, GS-050, GS-051, GS-052, GS-053, GS-054, GS-060, GS-066, GS-068, GS-069, GS-070, GS-076, GS-077, GS-078, GS-087, GS-090, GS-093, GS-096, GS-097, GS-098, GS-101, GS-102, GS-105, GS-110, GS-113, GS-115, GS-116, GS-118, GS-120, GS-122, GS-123, GS-132, GS-134, GS-145, GS-150, GS-151, GS-156, GS-163, GS-166, GS-167, GS-171, GS-173, GS-174, GS-175, GS-181, GS-182, GS-188, GS-191, GS-194, GS-195, GS-196, GS-198, GS-199, GS-202, GS-204, GS-205, GS-207, GS-210, GS-211, GS-213, GS-215, GS-217, GS-221, GS-223, GS-224, GS-227, GS-228, GS-229, GS-230, GS-231, GS-240, GS-243, GS-247, GS-253, GS-254, GS-255, GS-256, GS-257, GS-259, GS-261, GS-262, GS-264, GS-266, GS-267, GS-272, GS-276, GS-277, GS-278, GS-280, GS-281, GS-282, GS-284, GS-285, GS-286, GS-287, GS-289, GS-299, GS-305, GS-308, GS-309, GS-311, GS-314, GS-315, GS-323, GS-339, GS-341, GS-348, GS-350, GS-351, GS-362, GS-366, GS-368, GS-370, GS-372, GS-377, GS-378, GS-386, GS-387, GS-396, GS-397, GS-399, GS-401, GS-404, GS-405, GS-406, GS-409, GS-410, GS-411, GS-418, GS-419, GS-421, GS-422, GS-426, GS-427, GS-428, GS-429, GS-431, GS-434, GS-442, GS-444, GS-447, GS-458, GS-463, GS-464, GS-467, GS-470, GS-476, GS-477, GS-478, GS-479, GS-480, GS-481, GS-482, GS-483, GS-486, GS-487, GS-488, GS-489, GS-490, GS-491, GS-493, GS-494, GS-495, GS-497, GS-500, GS-501, GS-503, GS-505, GS-509, GS-510, GS-511, GS-512, GS-513, GS-514, GS-518, GS-520, GS-522, GS-525, GS-526, GS-528, GS-529, GS-530, GS-531, GS-532, GS-534, GS-535, GS-541, GS-542, GS-543, GS-544, GS-548, GS-549, GS-552, GS-555, GS-557, GS-559, GS-562, GS-564, GS-567, GS-568, GS-569, GS-570, GS-571, GS-572, GS-573, GS-574, GS-575, GS-576, GS-579, GS-580, GS-581, GS-583, GS-584, GS-585, GS-586, GS-587, GS-589, GS-592, GS-593, GS-594, GS-598, GS-602, GS-603, GS-604, GS-609, GS-610, GS-611, GS-612, GS-613, GS-614, GS-615, GS-616, GS-618, GS-620, GS-621, GS-622, GS-623, GS-625, GS-630, GS-633, GS-634, GS-635, or GS-637.

In some embodiments, the compound comprises one of the following, or a pharmaceutically acceptable salt thereof: GS-060, GS-066, GS-105, GS-171, GS-173, GS-181, GS-182, GS-227, GS-243, GS-255, GS-261, GS-262, GS-264, GS-272, GS-286, GS-287, GS-305, GS-309, GS-314, GS-315, GS-339, GS-348, GS-418, GS-442, GS-444, GS-490, GS-510, GS-552, GS-568, GS-570, GS-579, GS-589, GS-613, or GS-614.

In some embodiments, the compound comprises one of the following, or a pharmaceutically acceptable salt thereof: GS-171, GS-243, GS-255, GS-264, GS-286, GS-305, GS-442, GS-444, GS-568, GS-579, GS-589, GS-613, or GS-614.

### II. Synthesis and Testing of Compounds

The binding affinity of novel synthesized compounds described herein can be assessed using standard biophysical assays known in the art (e.g., isothermal titration calorimetry (ITC), surface plasmon resonance (SPR)). Cellular assays can then be used to assess the compound's ability to induce GSPT1 degradation and inhibit cancer cell proliferation. Besides evaluating a compound's induced changes in the protein levels of GSPT1. Assays suitable for use in any or all of these steps are known in the art, and include, e.g., western blotting, quantitative mass spectrometry (MS) analysis, flow cytometry, enzymatic activity assay, ITC, SPR, cell growth inhibition, xenograft, orthotopic, and patient-derived xenograft models. Suitable cell lines for use in any or all of these steps are known in the art and include HEL, RS4;11, MV4;11, MOLT-4, CCRF-CEM, Kasumi-1, MM.1S, HL-60, WSU-DLCL2, Pfeiffer, and SU-DHL-1 cancer cell lines. Suitable mouse models for use in any or all of these steps are known in the art and include subcutaneous xenograft models, orthotopic models, patient-derived xenograft models, and patient-derived orthotopic models.

By way of non-limiting example, detailed synthesis protocols are described in the Examples for specific exemplary compounds.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements.

In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

Pharmaceutically acceptable prodrugs of the compounds disclosed herein are contemplated and can be synthesized using conventional methods known in the art or methods corresponding to those described in the Examples (e.g., converting hydroxyl groups or carboxylic acid groups to ester groups). As used herein, a “prodrug” refers to a compound that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

In some aspects, provided herein is a method for identifying a compound which mediates degradation or reduction of GSPT1, the method comprising: providing a compound comprising an a CRBN binder; contacting the compound with a cell comprising a ubiquitin ligase and GSPT1; determining whether GSPT1 level is decreased in the cell; and identifying the compound as a compound which mediates degradation or reduction of GSPT1. In certain embodiments, the cell is a cancer cell. In certain embodiments, the cancer cell is a GSPT1-mediated cancer cell.

### III. Characterization of Exemplary Compounds

Specific exemplary compounds were first characterized using cell viability assays (see Example 24). In the assays, MV4;11 and MOLM-13 cells were treated with compounds for three days. The IC₅₀ values ranged from 1 nM to over 10 uM.

When combined with FLT3 inhibitor gilteritinib, the potency of some of the compounds was increased synergistically. Particularly, the potency of GSPT1 degraders GS-002, GS-005, and GS-006 combined with gilteritinib was significantly improved, as indicated by the cell growth inhibition of MOLM-13 cells. These experiment results demonstrated that the GSPT1 degraders can be used in a combination therapy with a second compound such as an FLT3 pathway inhibitor.

### IV. Pharmaceutical Compositions

In some aspects, the compositions and methods described herein include the manufacture and use of pharmaceutical compositions and medicaments that include one or more compounds (e.g. heterobifunctional compounds) as disclosed herein. Also included are the pharmaceutical compositions themselves. In some embodiments, the pharmaceutical composition comprises a compound described herein and a pharmaceutically acceptable excipient. In some embodiments, the pharmaceutical composition includes a compound described herein such as a GSPT1 degrader. In some embodiments, the pharmaceutical composition includes a second compound such as an FLT3 pathway inhibitor, a RAS-RAF-MEK-ERK pathway inhibitor, or a PI3K-AKT-mTOR pathway inhibitor or activator. The second compound may be an FLT3 pathway inhibitor or an FLT3 inhibitor such as Gilteritinib.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents or any other cancer treatment known in the art. When co-administered, compounds disclosed herein can operate in conjunction with conventional chemotherapeutic agents or any other cancer treatment known in the art to produce mechanistically additive or synergistic therapeutic effects.

In some aspects, the pH of the compositions disclosed herein can be adjusted with pharmaceutically acceptable acids, bases, or buffers to enhance the stability of the compound or its delivery form.

Pharmaceutical compositions typically include a pharmaceutically acceptable excipient, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable excipient, adjuvant, or vehicle is a substance that can be administered to a patient, together with a compound of the disclosure, and which does not compromise the pharmacological activity thereof and is nontoxic when administered in doses sufficient to deliver a therapeutic amount of the compound. Exemplary conventional nontoxic pharmaceutically acceptable excipients, adjuvants, and vehicles include, but not limited to, saline, solvents, dispersion media, coatings, antibacterial and antifingal agents, isotonic and absorption delaying agents, and the like, compatible with pharmaceutical administration.

In particular, pharmaceutically acceptable excipients, adjuvants, and vehicles that can be used in the pharmaceutical compositions of this disclosure include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

Depending on the dosage form selected to deliver the compounds disclosed herein, different pharmaceutically acceptable excipients, adjuvants, and vehicles may be used. In the case of tablets for oral use, pharmaceutically acceptable excipients, adjuvants, and vehicles may be used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

As used herein, the compounds disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a subject (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivatives thereof.

In some aspects, the pharmaceutical compositions disclosed herein can include an effective amount of one or more compounds. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer).

In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In one embodiment, the present disclosure relates to pharmaceutical formulation comprising: (a) a compound of FORMULA 1, or a pharmaceutically acceptable salt, hydrate, solvate, prodrug, stereoisomer, or tautomer thereof; and (b) a second therapeutic agent.

In one embodiment, the present disclosure relates to pharmaceutical formulation comprising: (a) a compound of FORMULA 1, or a pharmaceutically acceptable salt, hydrate, solvate, prodrug, stereoisomer, or tautomer thereof; and (b) one or more therapeutic agent(s).

In one embodiment, the present disclosure relates to a pharmaceutical formulation comprising a therapeutically effective amount of: (a) a compound of FORMULA 1, or a pharmaceutically acceptable salt, hydrate, solvate, prodrug, stereoisomer, or tautomer thereof; and (b) one or more therapeutic agent(s).

In one embodiment, the present disclosure relates to a combination comprising a therapeutically effective amount of (a)) a compound of FORMULA 1, or a pharmaceutically acceptable salt, hydrate, solvate, prodrug, stereoisomer, or tautomer thereof; and (b) a second therapeutic agent.

In one embodiment, the present disclosure relates to a combination comprising a therapeutically effective amount of (a) a compound selected from Compound GS-001 to GS-639, or a pharmaceutically acceptable salt, hydrate, solvate, prodrug, stereoisomer, or tautomer thereof; and (b) a second therapeutic agent.

In one embodiment, the present disclosure relates to a combination comprising a therapeutically effective amount of (a) a compound selected from Compound GS-001 to GS-639, or a pharmaceutically acceptable salt, hydrate, solvate, prodrug, stereoisomer, or tautomer thereof; and (b) one or more therapeutic agent(s).

In one embodiment, the present disclosure relates to a combination comprising a therapeutically effective amount of (a) a compound selected from Compound GS-001 to GS-639, or a pharmaceutically acceptable salt, hydrate, solvate, prodrug, stereoisomer, or tautomer thereof; (b) a second therapeutic agent; and (c) a pharmaceutically acceptable carrier or excipient.

In one embodiment, the present disclosure relates to a combination comprising a therapeutically effective amount of (a) a compound selected from Compound GS-001 to GS-639, or a pharmaceutically acceptable salt, hydrate, solvate, prodrug, stereoisomer, or tautomer thereof; (b) one or more therapeutic agent(s); and (c) a pharmaceutically acceptable carrier or excipient.

In one embodiment, the present disclosure relates to a pharmaceutical formulation comprising a therapeutically effective amount of (a) a compound selected from Compound GS-001 to GS-639, or a pharmaceutically acceptable salt, hydrate, solvate, prodrug, stereoisomer, or tautomer thereof; and (b) a second therapeutic agent.

In one embodiment, the present disclosure relates to a pharmaceutical formulation comprising a therapeutically effective amount of (a) a compound selected from Compound GS-001 to GS-639, or a pharmaceutically acceptable salt, hydrate, solvate, prodrug, stereoisomer, or tautomer thereof; and (b) one or more therapeutic agent(s).

In one embodiment, the present disclosure relates to a pharmaceutical formulation comprising a therapeutically effective amount of (a) a compound selected from Compound GS-001 to GS-639, or a pharmaceutically acceptable salt, hydrate, solvate, prodrug, stereoisomer, or tautomer thereof; (b) one or more therapeutic agent; and (c) a pharmaceutically acceptable carrier or excipient.

In one embodiment, the second or more therapeutic agent(s) is/are an anti-cancer drug. In one embodiment, the second or more therapeutic agent(s) is/are an anti-proliferative agent. In one embodiment, the second or more therapeutic agent(s) is/are an immunomodulatory agent. In one embodiment, the second or more therapeutic agent(s) is a kinase inhibitor or activator. In one embodiment, the second or more therapeutic agent(s) is a kinase inhibitor.

In one embodiment, the second or more therapeutic agent(s) inhibits a FMS-like tyrosine kinase 3 gene (FLT3) pathway. In one embodiment, the second or more therapeutic agent(s) inhibits FLT3. In some embodiments, the second compound is an FLT3 pathway inhibitor. The second compound may be Gilteritinib.

In one embodiment, the second or more therapeutic agent(s) inhibits PI3K/AKT/mTOR pathway. In one embodiment, the second or more therapeutic agent(s) inhibits PI3K. In one embodiment, the second or more therapeutic agent(s) inhibits AKT. In one embodiment, the second or more therapeutic agent(s) inhibits mTOR. In some embodiments, the second compound is a PI3K-AKT-mTOR pathway inhibitor or activator.

In one embodiment, the second or more therapeutic agent(s) inhibits MAPK pathway. In one embodiment, the second or more therapeutic agent(s) inhibits RAS/RAF/MEK/ERK pathway. In one embodiment, the second or more therapeutic agent(s) inhibits RAS. In one embodiment, the second or more therapeutic agent(s) inhibits RAF. In one embodiment, the second or more therapeutic agent(s) inhibits MEK. In one embodiment, the second or more therapeutic agent(s) inhibits ERK. In some embodiments, the second compound is a RAS-RAF-MEK-ERK pathway inhibitor.

### V. Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

The pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

In some embodiments, the pharmaceutical compositions of this disclosure are orally administered in any orally acceptable dosage form including, but not limited to, capsules, tablets, emulsions and aqueous suspensions, dispersions and solutions.

In some embodiments, the pharmaceutical compositions of this disclosure are administered in the form of suppositories for rectal administration. These compositions can be prepared by mixing a compound of this disclosure with a suitable non-irritating excipient which is solid at room temperature (rt) but liquid at the rectal temperature and therefore will melt in the rectum to release the active components. Such materials include, but are not limited to, cocoa butter, beeswax, and polyethylene glycols.

In some embodiments, the pharmaceutical compositions of this disclosure are administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

In some embodiments, the pharmaceutical compositions of this disclosure are administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this disclosure can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of the compounds described herein and one or more additional compounds (e.g., one or more additional compounds, drugs, or agents used for the treatment of cancer or any other condition or disease, including conditions or diseases known to be associated with or caused by cancer), both the compounds and the additional compounds may be present at dosage levels of between about 1 to 100%, and more preferably between about 5 to 95% of the dosage normally administered in a monotherapy regimen. The additional agents can be administered separately, as part of a multiple dose regimen, from the compounds ofthis disclosure. Alternatively, those agents can be part of a single dosage form, mixed together with the compounds of this disclosure in a single composition.

In some aspects, the pharmaceutical compositions disclosed herein can be included in a container, pack, or dispenser together with instructions for administration.

### VI. Methods of Treatment

Disclosed herein, in some embodiments, are methods of administering a composition described herein to a subject. Some embodiments relate to use a composition described herein, such as administering the composition to a subject. Some embodiments relate to a method of treating a disorder in a subject in need thereof. Some embodiments relate to use of a composition described herein in the method of treatment. In some embodiments, the method of treatment comprises administering to a subject in need thereof, a first compound comprising a GSPT1 degrader and a second compound comprising an FLT3 pathway inhibitor, a RAS-RAF-MEK-ERK pathway inhibitor, or a PI3K-AKT-mTOR pathway inhibitor or activator. Some embodiments include administering a composition described herein to a subject with the disorder. In some embodiments, the administration treats the disorder in the subject. In some embodiments, the composition treats the disorder in the subject. In some embodiments, the treatment comprises prevention, inhibition, or reversion of the disorder in the subject. In some embodiments, the subject has cancer.

According to one aspect of the present disclosure, a method of treating a GSPT1-mediated disease disclosed herein comprises administering to a subject with GSPT1-mediated disease the compound or a pharmaceutically acceptable salt or analog thereof.

The compound may be a compound described herein, such as a compound of FORMULA 1. In some embodiments, the compound is a GSPT1 degrader. In one embodiment, the compound is selected from the group consisting of GS-001 to GS-639, or analogs thereof. In some embodiments, the first compound is a compound disclosed in Table 1, Table 2, or Table 3, a pharmaceutically acceptable salt or solvate thereof. The compound may be included in a pharmaceutical composition described herein. In some embodiments, the first compound and/or the second compound is administered to the subject as a pharmaceutical composition comprising a pharmaceutically acceptable carrier.

The methods disclosed herein contemplate administration of an effective amount of a compound or composition to achieve the desired or stated effect. Typically, the compounds or compositions of the disclosure will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound. In some embodiments, the first compound and the second compound are coadministered to the subject. In some embodiments, the first compound and the second compound are each administered separately to the subject. In some embodiments, the subject is a mammal. In some embodiments, the subject is a human.

In some aspects, provided herein is a compound described herein for preventing or treating a disease or condition. In some aspects, provided herein is a heterobifunctional compound described herein for preventing or treating a disease or condition.

In some aspects, provided herein is a compound described herein for treating or preventing one or more diseases or conditions disclosed herein in a subject in need thereof. In certain embodiments, the disease or condition is a GSPT1-mediated disease or condition. In certain embodiments, the diseases or conditions are cancer, inflammation, auto-immune disease, viral infections, and immunological diseases. In one embodiment, the GSPT1-mediated cancer is selected from the group consisting of brain cancer, stomach cancer, gastrointestinal tract cancer, liver cancer, biliary passage cancer, breast cancer, ovary cancer, cervix cancer, prostate cancer, testis cancer, penile cancer, genitourinary tract cancer, esophagus cancer, larynx cancer, skin cancer, lung cancer, pancreas cancer, thyroid cancer, gland cancer, bladder cancer, kidney cancer, muscle cancer, bone cancer, cancers of the hematopoietic system, myeloproliferative neoplasms, essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hypereosinophilic syndrome, cutaneous T-cell lymphoma, B-cell lymphoma, and myeloma. In some embodiments, the disease or condition includes a cancer. In some embodiments, the disease or condition includes a GSPT1-mediated cancer.

In one embodiment, the GSPT1-mediated cancer is selected from the group consisting of mesothelioma, leukemias, and lymphomas such as cutaneous T-cell lymphomas (CTCL), noncutaneous peripheral T-cell lymphomas, lymphomas associated with human T-cell lymphotrophic vims (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma, myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, and nasopharyngeal), esophageal cancer, genitourinary cancers (e.g., prostate, bladder, renal, uterine, ovarian, testicular), lung cancer (e.g., small-cell and non-small cell), breast cancer, pancreatic cancer, melanoma, and other skin cancers, stomach cancer, brain tumors, tumors related to Gorlin's syndrome (e.g., medulloblastoma, meningioma, etc.), liver cancer, non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST), cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

In one embodiment, the GSPT1-mediated disease is a relapsed cancer.

In one embodiment, the GSPT1-mediated disease is refractory to one or more previous treatments.

In some aspects, provided herein are use of a compound in manufacture of a medicament for preventing or treating one or more diseases or conditions disclosed herein. The medicament may include a compound described herein. The medicament may include a pharmaceutical composition described herein.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had, having an elevated risk to have, or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition disclosed herein (e.g., an GSPT1-mediated disease). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the compounds, compositions and methods of the present disclosure. In some embodiments, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present disclosure. In certain embodiments, the subject has an elevated risk of developing one or more GSPT1-mediated diseases. Exemplary GSPT1-mediated diseases that can be treated with compounds include, for example, cancers of brain, stomach, gastrointestinal tracts, liver, biliary passage, breast, ovary, cervix, prostate, testis, penile, genitourinary tract, esophagus, larynx, skin, lung, pancreas, thyroid, glands, bladder, kidney, muscle, bone, and cancers of the hematopoietic system, such as myeloproliferative neoplasms, including essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hypereosinophilic syndrome, cutaneous T-cell lymphoma, B-cell lymphoma, myeloma, and other hematologic malignancies.

In some embodiments, the compound is more efficacious for treating a condition such as a cancer than an existing drug. For example, the compound may be effective at a lower dose than the existing drug. In some embodiments, the compound is more efficacious than a known cereblon modulator. In some embodiments, the compound is more efficacious than CC-90009. In some embodiments, the compound is more efficacious than a known drug at reducing cell viability. For example, the compound may be more efficacious than CC-90009 at reducing cancer cell viability. Some such embodiments are included in Table 2, and Table 3.

In some embodiments, the compound in combination with a second compound such as an FLT3 pathway inhibitor, a RAS-RAF-MEK-ERK pathway inhibitor, a PI3K-AKT-mTOR pathway inhibitor or activator is more efficacious for treating a condition such as cancer than an existing drug. The second compound may be an FLT3 pathway inhibitor or an FLT3 inhibitor. In some embodiments, the second compound comprises an FLT3 pathway inhibitor. In some embodiments, the second compound comprises an FLT3 inhibitor. In some embodiments, the FLT3 inhibitor comprises Gilteritinib, Midostaurin, Sorafenib, Sunitinib, or Lestaurtinib. In some embodiments, the FLT3 inhibitor comprises Gilteritinib. In some embodiments, the compound in combination with the second compound is effective at a lower dose than the existing drug. In some embodiments, the compound in combination with the second compound is more efficacious than a known cereblon modulator. In some embodiments, the compound in combination with the second compound is more efficacious than CC-90009. In some embodiments, the compound in combination with the second compound is more efficacious than a known drug at reducing cell viability. For example, the compound in combination with the second compound may be more efficacious than CC-90009 at reducing cancer cell viability. Some such embodiments are included in Table 2 and Table 3. In some embodiments, the increased efficacy of the compound in combination with the second compound, compared to another drug, is achieved.

In some embodiments, the second compound comprises a RAS-RAF-MEK-ERK pathway inhibitor. In some embodiments, the RAS-RAF-MEK-ERK pathway inhibitor comprises Vemurafenib, Dabrafenib, Encorafenib, SB590885, PLX4720, XL281, RAF265, Trametinib, Binimetinib, Cobimetinib, Selumetinib, CI-1040, or PD0325901. In some embodiments, the second compound comprises a PI3K-AKT-mTOR pathway inhibitor or activator. In some embodiments, the PI3K-AKT-mTOR pathway inhibitor or activator comprises Apitolisib, Idelalisib, Copanlisib, Duvelisib, MK-2206, ARQ-092, gedatolisib, Apitolisib, VQD-002, Perifosine, AZD5363, Ipatasertib, Rapamycin, temsirolimus, everolimus, ridaforolimus, Rapalogs, Sirolimus, dactolisib, BGT226, SF1126, PKI-587, NVPBE235, sapanisertib, AZD8055, and AZD2014, Wortmannin, LY294002, hibiscone C, Taselisib, Perifosine, Buparlisib, Umbralisib, PX-866, Dactolisib, CUDC-907, Voxtalisib, bisper oxovanadium, or Sarcopoterium.

In some embodiments, the compounds disclosed herein can selectively affect GSPT1-mediated disease cells compared to WT (wild type) cells (i.e., a heterobifunctional compound able to kill or inhibit the growth of an GSPT1-mediated disease cell while also having a relatively low ability to lyse or inhibit the growth of a WT cell), e.g., possess a GI₅₀ for one or more GSPT1-mediated disease cells more than 1.5-fold lower, more than 2-fold lower, more than 2.5-fold lower, more than 3-fold lower, more than 4-fold lower, more than 5-fold lower, more than 6-fold lower, more than 7-fold lower, more than 8-fold lower, more than 9-fold lower, more than 10-fold lower, more than 15-fold lower, or more than 20-fold lower than its GI₅₀ for one or more WT cells, e.g., WT cells of the same species and tissue type as the GSPT1-mediated disease cells.

In some embodiments, the compound has an IC₅₀ below that of CC-90009 in cells. In some embodiments, the cells include a cell line. In some cases, the cell line includes MV4;11 cells. In some cases, the cell line includes MOLM-13 cells. In some cases, the IC₅₀ of the compound is determined based on treating the cells with the compound alone. In some cases, the IC₅₀ of the compound is below 100 nM. In some cases, the IC₅₀ of the compound is below 100 nM. In some cases, the IC₅₀ of the compound is below 75 nM. In some cases, the IC₅₀ of the compound is below 50 nM. In some cases, the IC₅₀ of the compound is below 25 nM. In some cases, the IC₅₀ of the compound is below 10 nM. In some cases, the IC₅₀ of the compound is below 9 nM. In some cases, the IC₅₀ of the compound is below 8 nM. In some cases, the IC₅₀ of the compound is below 7 nM. In some cases, the IC₅₀ of the compound is below 6 nM. In some cases, the IC₅₀ of the compound is below 5 nM. In some cases, the IC₅₀ of the compound is below 4 nM. In some cases, the IC₅₀ of the compound is below 3 nM. In some cases, the IC₅₀ of the compound is below 2 nM. In some cases, the IC₅₀ of the compound is below 1 nM. In some cases, the IC₅₀ of the compound is below 0.5 nM. In some cases, the IC₅₀ of the compound is below 0.25 nM. In some cases, the IC₅₀ of the compound is above 100 nM. In some cases, the IC₅₀ of the compound is above 100 nM. In some cases, the IC₅₀ of the compound is above 75 nM. In some cases, the IC₅₀ of the compound is above 50 nM. In some cases, the IC₅₀ of the compound is above 25 nM. In some cases, the IC₅₀ of the compound is above 10 nM. In some cases, the IC₅₀ of the compound is above 9 nM. In some cases, the IC₅₀ of the compound is above 8 nM. In some cases, the IC₅₀ of the compound is above 7 nM. In some cases, the IC₅₀ of the compound is above 6 nM. In some cases, the IC₅₀ of the compound is above 5 nM. In some cases, the IC₅₀ of the compound is above 4 nM. In some cases, the IC₅₀ of the compound is above 3 nM. In some cases, the IC₅₀ of the compound is above 2 nM. In some cases, the IC₅₀ of the compound is above 1 nM. In some cases, the IC₅₀ of the compound is above 0.5 nM. In some cases, the IC₅₀ of the compound is above 0.5 nM. In some cases, the IC₅₀ of the compound is above 0.25 nM. Some examples of IC₅₀ values and ranges for compounds are shown in Table 2 and/or Table 3.

In some embodiments, the compound in combination with a second compound such as an FLT3 pathway inhibitor, a RAS-RAF-MEK-ERK pathway inhibitor, a PI3K-AKT-mTOR pathway inhibitor or activator, has an IC₅₀ below that of CC-90009 in cells. The second compound may be an FLT3 pathway inhibitor or an FLT3 inhibitor such as Gilteritinib. In some embodiments, the cells include a cell line. In some cases, the cell line includes MV4;11 cells. In some cases, the cell line includes MOLM-13 cells. In some cases, the IC₅₀ is determined based on treating the cells with the compound in combination with the second compound (e.g. gilteritinib). In some cases, the IC₅₀ of the compound in combination with the second compound is below 100 nM. In some cases, the IC₅₀ of the compound in combination with the second compound is below 100 nM. In some cases, the IC₅₀ of the compound in combination with the second compound is below 75 nM. In some cases, the IC₅₀ of the compound in combination with the second compound is below 50 nM. In some cases, the IC₅₀ of the compound in combination with the second compound is below 25 nM. In some cases, the IC₅₀ of the compound in combination with the second compound is below 10 nM. In some cases, the IC₅₀ of the compound in combination with the second compound is below 9 nM. In some cases, the IC₅₀ of the compound in combination with the second compound is below 8 nM. In some cases, the IC₅₀ of the compound in combination with the second compound is below 7 nM. In some cases, the IC₅₀ of the compound in combination with the second compound is below 6 nM. In some cases, the IC₅₀ of the compound in combination with the second compound is below 5 nM. In some cases, the IC₅₀ of the compound in combination with the second compound is below 4 nM. In some cases, the IC₅₀ of the compound in combination with the second compound is below 3 nM. In some cases, the IC₅₀ of the compound in combination with the second compound is below 2 nM. In some cases, the IC₅₀ of the compound in combination with the second compound is below 1 nM. In some cases, the IC₅₀ of the compound in combination with the second compound is below 0.5 nM. In some cases, the IC₅₀ of the compound in combination with the second compound is below 0.25 nM. In some cases, the IC₅₀ of the compound is above 100 nM. In some cases, the IC₅₀ of the compound is above 100 nM. In some cases, the IC₅₀ of the compound is above 75 nM. In some cases, the IC₅₀ of the compound is above 50 nM. In some cases, the IC₅₀ of the compound is above 25 nM. In some cases, the IC₅₀ of the compound is above 10 nM. In some cases, the IC₅₀ of the compound is above 9 nM. In some cases, the IC₅₀ of the compound is above 8 nM. In some cases, the IC₅₀ of the compound is above 7 nM. In some cases, the IC₅₀ of the compound is above 6 nM. In some cases, the IC₅₀ of the compound is above 5 nM. In some cases, the IC₅₀ of the compound is above 4 nM. In some cases, the IC₅₀ of the compound is above 3 nM. In some cases, the IC₅₀ of the compound is above 2 nM. In some cases, the IC₅₀ of the compound is above 1 nM. In some cases, the IC₅₀ of the compound is above 0.5 nM. In some cases, the IC₅₀ of the compound is above 0.5 nM. In some cases, the IC₅₀ of the compound is above 0.25 nM. Some examples of IC₅₀ values for compounds in combination with a second compound are shown in Table 2 and Table 3.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The method may include administering the compound to the subject by any route of administration described herein. In one embodiment, the compound is administered to the subject orally, parenterally, intradermally, subcutaneously, topically, or rectally.

In one embodiment, the method further comprises administering to the subject an additional therapeutic regimen for treating cancer, inflammatory disorders, or autoimmune diseases.

In one embodiment, the additional therapeutic regimen is selected from the group consisting of surgery, chemotherapy, radiation therapy, hormone therapy, targeted therapy, and immunotherapy.

Some embodiments include administering an additional compound. Such additional compounds may preferably be a kinase inhibitor, in particular, FLT3 pathway inhibitor (e.g. Gilteritinib, Midostaurin, Sorafenib, Sunitinib, or Lestaurtinib), MAPK pathway inhibitor, RAS-RAF-MEK-ERK pathway inhibitor (e.g. Vemurafenib, Dabrafenib; Encorafenib, SB590885, PLX4720, XL281, RAF265, Trametinib, Binimetinib, Cobimetinib, Selumetinib, CI-1040, or PD0325901), or PI3K-AKT-mTOR pathway inhibitor or activators (e.g. Apitolisib, Idelalisib, Copanlisib, Duvelisib, MK-2206, ARQ-092, gedatolisib, Apitolisib, VQD-002, Perifosine, AZD5363, Ipatasertib, Rapamycin, temsirolimus, everolimus, ridaforolimus, Rapalogs, Sirolimus, dactolisib, BGT226, SF1126, PKI-587, NVPBE235, sapanisertib, AZD8055, and AZD2014, Wortmannin, LY294002, hibiscone C, Taselisib, Perifosine, Buparlisib, Umbralisib, PX-866, Dactolisib, CUDC-907, Voxtalisib, bisper oxovanadium, or Sarcopoterium).

Some embodiments include administering to a subject in need thereof a first compound. In some embodiments, the first compound is a GSPT1 degrader. In some embodiments, the first compound is a compound of FORMULA 1. In some embodiments, the first compound comprises any one of the compounds in Table 1, Table 2, or Table 3. In some embodiments, the first compound is selected from GS-001 to GS-639, or a pharmaceutically acceptable salt thereof. In some embodiments, the first compound includes GS-002 or a pharmaceutically acceptable salt thereof. In some embodiments, the first compound includes GS-005 or a pharmaceutically acceptable salt thereof. In some embodiments, the first compound includes GS-006 or a pharmaceutically acceptable salt thereof. In some embodiments, the first compound does not bind JAK. In some embodiments, the first compound includes a truncated JAK binding moiety. In some embodiments, the first compound does not include a JAK binding moiety. Some embodiments include administering to the subject a second compound comprising an FLT3 pathway inhibitor, a RAS-RAF-MEK-ERK pathway inhibitor, a PI3K-AKT-mTOR pathway inhibitor or activator. In some embodiments, the second compound includes an FLT3 pathway inhibitor. In some embodiments, the second compound includes a RAS-RAF-MEK-ERK pathway inhibitor. In some embodiments, the second compound includes a PI3K-AKT-mTOR pathway inhibitor or activator. Some embodiments include a method of treatment, comprising: administering to a subject in need thereof, a first compound comprising a GSPT1 degrader and a second compound comprising an FLT3 pathway inhibitor, a RAS-RAF-MEK-ERK pathway inhibitor, a PI3K-AKT-mTOR pathway inhibitor or activator. In some embodiments, the first compound and/or the second compound is administered to the subject as a pharmaceutical composition comprising a pharmaceutically acceptable carrier. In some embodiments, the first compound and the second compound are coadministered to the subject. In some embodiments, the first compound and the second compound are each administered separately to the subject.

Some embodiments include a method of treating or preventing cancer comprising administering to a patient in need thereof a compound that has degrader activity for GSPT1 in combination with one or more additional therapeutic agents, wherein the additional therapeutic agent is selected from an inhibitor of an inhibitory molecule, an activator of a costimulatory molecule, a chemotherapeutic agent, a targeted anti-cancer therapy, an oncolytic drug, a cytotoxic agent, or combination thereof.

In one embodiment, the present disclosure relates to a method of treating or preventing cancer comprising administering to a patient in need thereof a combination comprising (a) a compound of FORMULA 1, or a pharmaceutically acceptable salt, hydrate, solvate, prodrug, stereoisomer, or tautomer thereof; and (b) a second therapeutic agent.

In one embodiment, the present disclosure relates to a method of treating or preventing cancer comprising administering to a patient in need thereof a combination comprising (a) a compound of FORMULA 1, or a pharmaceutically acceptable salt, hydrate, solvate, prodrug, stereoisomer, or tautomer thereof; and (b) one or more therapeutic agent.

In one embodiment, the present disclosure relates to a method of treating or preventing cancer comprising administering to a patient in need thereof a combination comprising (a) a compound selected from Compound GS-001 to GS-639, or a pharmaceutically acceptable salt, hydrate, solvate, prodrug, stereoisomer, or tautomer thereof; and (b) a second therapeutic agent.

In one embodiment, the present disclosure relates to a method of treating or preventing cancer comprising administering to a patient in need thereof a combination comprising (a) a compound selected from Compound GS-001 to GS-639, or a pharmaceutically acceptable salt, hydrate, solvate, prodrug, stereoisomer, or tautomer thereof; and (b) one or more therapeutic agent.

In some embodiments, the second or more therapeutic agent(s) inhibits a FMS-like tyrosine kinase 3 gene (FLT3) pathway. In one embodiment, the second or more therapeutic agent(s) inhibits FLT3. Examples of FLT3 inhibitors include Gilteritinib, Midostaurin, Sorafenib, Sunitinib, or Lestaurtinib. In some embodiments, the FLT3 inhibitor includes Gilteritinib. In some embodiments, the second compound includes an FLT3 pathway inhibitor (e.g. Gilteritinib).

In some embodiments, the second or more therapeutic agent(s) inhibits PI3K/AKT/mTOR pathway. In one embodiment, the second or more therapeutic agent(s) inhibits PI3K. In one embodiment, the second or more therapeutic agent(s) inhibits AKT. In one embodiment, the second or more therapeutic agent(s) inhibits mTOR. In some embodiments, the second compound includes a PI3K-AKT-mTOR pathway inhibitor or activator. Examples of RAS-RAF-MEK-ERK pathway inhibitors include Vemurafenib, Dabrafenib; Encorafenib, SB590885, PLX4720, XL281, RAF265, Trametinib, Binimetinib, Cobimetinib, Selumetinib, CI-1040, or PD0325901. In some embodiments, the second compound includes a RAS-RAF-MEK-ERK pathway inhibitor.

In some embodiments, the second or more therapeutic agent(s) inhibits MAPK pathway. In one embodiment, the second or more therapeutic agent(s) inhibits RAS/RAF/MEK/ERK pathway. In one embodiment, the second or more therapeutic agent(s) inhibits RAS. In one embodiment, the second or more therapeutic agent(s) inhibits RAF. In one embodiment, the second or more therapeutic agent(s) inhibits MEK. In one embodiment, the second or more therapeutic agent(s) inhibits ERK. In some embodiments, the second compound includes a RAS-RAF-MEK-ERK pathway inhibitor. Examples of PI3K-AKT-mTOR pathway inhibitors or activators include Apitolisib, Idelalisib, Copanlisib, Duvelisib, MK-2206, ARQ-092, gedatolisib, Apitolisib, VQD-002, Perifosine, AZD5363, Ipatasertib, Rapamycin, temsirolimus, everolimus, ridaforolimus, Rapalogs, Sirolimus, dactolisib, BGT226, SF1126, PKI-587, NVPBE235, sapanisertib, AZD8055, and AZD2014, Wortmannin, LY294002, hibiscone C, Taselisib, Perifosine, Buparlisib, Umbralisib, PX-866, Dactolisib, CUDC-907, Voxtalisib, bisper oxovanadium, Sarcopoterium. In some embodiments, the second compound includes a PI3K-AKT-mTOR pathway inhibitor. In some embodiments, the second compound includes a PI3K-AKT-mTOR pathway activator.

### VII. Definition of Terms

As used herein, the terms “comprising” and “including” are used in their open, non-limiting sense.

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

The term “allyl,” as used herein, refers a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkoxy”, as used herein, means an alkyl group as defined herein which is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “aryl”, as used herein, “refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hückel theory. In certain embodiments, an aryl comprises six to fourteen carbon atoms (C₆-C₁₄ aryl or 6-14 membered aryl). In certain embodiments, an aryl comprises six to ten carbon atoms (C₆-C₁₀ aryl or 6-10 membered aryl). Examples of such groups include, but are not limited to, phenyl, fluorenyl and naphthyl. The terms “Ph” and “phenyl,” as used herein, mean a —C₆H₅ group.

The term “heteroaryl”, refers to a radical derived from a 3- to 18-membered aromatic ring radical (i.e. 3-18 membered heteroaryl) that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. In certain embodiments, a heteroaryl refers to a radical derived from a 3- to 10-membered aromatic ring radical (3-10 membered heteroaryl). In certain embodiments, a heteroaryl refers to a radical derived from 5- to 7-membered aromatic ring (5-7 membered heteroaryl). Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, a heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclyl”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from 0, S and N, and with the proviso that the ring of said group does not contain two adjacent 0 atoms or two adjacent S atoms. A heterocyclyl group may include fused, bridged or spirocyclic ring systems. In certain embodiments, a hetercyclyl group comprises 3 to 10 ring atoms (3-10 membered heterocyclyl). In certain embodiments, a hetercyclyl group comprises 3 to 8 ring atoms (3-8 membered heterocyclyl). In certain embodiments, a hetercyclyl group comprises 4 to 10 ring atoms (4-10 membered heterocyclyl). In certain embodiments, a hetercyclyl group comprises 4 to 8 ring atoms (4-8 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to provide either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8, diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” or “carbocyclyl” means a saturated, monocyclic, bicyclic, tricyclic, or tetracyclic radical having a total of from 4, 5, 6, 7, 8, 9, 10, 11, 12, or 13 carbon atoms in its ring system. A carbocyclyl may be fused, bridged or spirocyclic. In certain embodiments, a carbocyclyl comprises 3 to 8 carbon ring atoms (3-8 membered carbocyclyl). In certain embodiments, a carbocyclyl comprises 3 to 10 carbon ring atoms (3-10 membered carbocyclyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C≡N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-carbocyclyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “length” when refers to a moiety means the smallest number of carbon and/or hetero atoms from one end to the other end of the moiety. When it refers to the linker, it means the smallest number of atoms from the end connects to the TRK ligand and the end connects to the Degradation Tag. It applies to both situations where the linker is linear or branched, and where the linker comprises a ring system.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “null” means the absence of an atom or moiety, and there is a bond between adjacent atoms in the structure.

The term “optionally substituted” means that the specified group may be either unsubstituted or substituted by one or more substituents as defined herein. It is to be understood that in the compounds of the present disclosure when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present disclosure is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the CO aryl has 3 carbon atoms remaining that are unsubstituted. Those three unsubstituted carbon atoms are each bound to one hydrogen atom to fill their valencies. Unless otherwise specified, an optionally substituted radical may be a radical unsubstituted or substituted with one or more substituents selected from halogen, CN, NO₂, OR^{m}, SR^{m}, NRⁿR^{o}, COR^{m}, CO₂R^{m}, CONRⁿR^{o}, SOR^{m}, SO₂R^{m}, SO₂NRⁿR^{o}, NRⁿCOR^{o}, NR^{m}C(O)NRⁿR^{o}, NRⁿSOR^{o}, NRⁿSO₂R^{o}, C₁-C₈ alkyl, C₁-C₈alkoxyC₁-C₈alkyl, C₁-C₈ haloalkyl, C₁-C₈ hydroxyalkyl, C₁-C₈alkylaminoC₁-C₈ alkyl, C₃-C₇ carbocyclyl, 3-7 membered heterocyclyl, C₂-C₈ alkenyl, C₂-C₈ alkynyl, aryl, and heteroaryl, wherein R^{m}, Rⁿ, and R^{o} are independently selected from null, hydrogen, C₁-C₈ alkyl, C₂-C₈ alkenyl, C₂-C₈ alkynyl, C₃-C₇ carbocyclyl, 3-7 membered heterocyclyl, aryl, and heteroaryl, or Rⁿ and R^{o} together with the atom to which they are connected form a 3-8 membered carbocyclyl or heterocyclyl ring.

As used herein, the same symbol in different FORMULA means different definition, for example, the definition of R1 in FORMULA 1 is as defined with respect to FORMULA 1 and the definition of R1 in FORMULA 6 is as defined with respect to FORMULA 6.

As used herein, each unit in the linker moiety

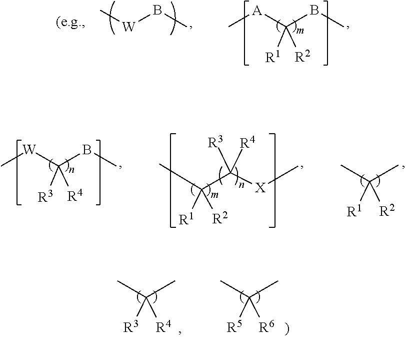

can be the same as or different from each other. In certain embodiments, each unit in the linker moiety is the same as each other.

As used herein, when m (or n or o or p) is defined by a range, for example, “m is 0 to 15” or “m=0-3” mean that m is an integer from 0 to 15 (i.e. in is 0, 1, 2, 3, 4, 5, 6, 7, 8, 9, 10, 11, 12, 13, 14, or 15) or m is an integer from 0 to 3 (i.e. m is 0, 1, 2, or 3) or is any integer in the defined range.

The term “combination therapy” or “combination” or “in combination with” refers to the administration of two or more therapeutic agents to treat a condition or disorder described in the present disclosure (e.g., cancer). Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e.. the effect achieved when the active ingredients used together is greater than the sum of the effects that results from using the compounds separately. A synergistic effect can be attained when the active ingredients are: (1) co formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

A “therapeutic agent” as used herein refers to a therapy, e.g., a molecule, including but not limited to, a chemical compound, peptide, antibody, antibody fragment, antibody conjugate, or nucleic acid; a gene or cell therapy; or a radiation therapy, which is therapeutically active or enhances the therapeutic activity when administered to a patient in combination with a compound of the present disclosure or which reduces one or more side effects of the compound of the present disclosure when administered to a patient in combination with a compound of the present disclosure.

“Cancer” means any cancer caused by the uncontrolled proliferation of aberrant cells, such as tumors, neoplasms, carcinomas, sarcomas, leukemias, lymphomas, and the like. Cancer cells can spread locally or through the bloodstream and lymphatic system to other parts of the body. For example, cancers include, but are not limited to, mesothelioma, leukemias, and lymphomas such as cutaneous T-cell lymphomas (CTCL), noncutaneous peripheral T-cell lymphomas, lymphomas associated with human T-cell lymphotrophic vims (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, and nasopharyngeal), esophageal cancer, genitourinary cancers (e.g., prostate, bladder, renal, uterine, ovarian, testicular), lung cancer (e.g., small-cell and non-small cell), breast cancer, pancreatic cancer, melanoma, and other skin cancers, stomach cancer, brain tumors, tumors related to Gorlin's syndrome (e.g., medulloblastoma, meningioma, etc.), liver cancer, non-small cell lung cancer (NSCLC), melanoma, triple-negative breast cancer (TNBC), nasopharyngeal cancer (NPC), microsatellite stable colorectal cancer (mssCRC), thymoma, carcinoid, and gastrointestinal stromal tumor (GIST). Additional exemplary forms of cancer which may be treated by the compounds and compositions described herein include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

The second agent can be an anti-cancer agent. The term “anti-cancer” or “anti-cancer agent” pertains to an agent which treats a cancer (i.e., a compound, antibody, etc. which is useful in the treatment of a cancer). The anti-cancer effect may arise through one or more mechanisms, including, but not limited to, the regulation of cell growth or proliferation, the inhibition of angiogenesis (the formation of new blood vessels), the inhibition of metastasis (the spread of a tumor from its origin), the inhibition of invasion (the spread of tumor cells into neighboring normal structures), the inhibition of a checkpoint molecule, or the promotion of apoptosis.

The anti-cancer agent is can be an anti-proliferative agent or an immunomodulatory agent. In one embodiment, the second agent is an immunomodulatory agent.

The term “antiproliferative” or “antiproliferative agent” as used herein pertains to an agent, which inhibits cell growth or cell proliferation. The anti-proliferative agent can be a cytotoxic agent (e.g., alkylating agent, antimetabolites, etc.), a targeted agent (e.g., EGF inhibitor, Tyrosine protein kinase inhibitor, angiogenesis inhibitor, etc.), or a hormonal agent (e.g., estrogens selective estrogen receptor modulators, etc.). Examples of antiproliferative agents include alkylating agents, anti-metabolites, an antibiotic, a detoxifying agent, an EGFR inhibitor, a HER2 inhibitor, a histone deacetylase inhibitor, a hormone, a mitotic inhibitor, an MTOR inhibitor, a multi-kinase inhibitor, a serine/threonine inhibitor, a tyrosine kinase inhibitor, a VEGF/VEGFR inhibitor; a taxane or taxane derivative, an aromatase inhibitor, an anthracycline, a microtubule targeting drug, a topoisomerase poison drug, an inhibitor of a molecular target or enzyme.

The term “immunomodulatory agent” is agent that modifies the immune response or the functioning of the immune system (as by the stimulation of antibody formation or the inhibition of white blood cell activity). The immunomodulatory agents can be an immunomodulator, a cytokine, a vaccine, or an anti-body.

The term “immunomodulator” is an inhibitor of an immune checkpoint molecule.

Additional cancers that the compounds and compositions described herein may be useful in preventing, treating, and studying are, for example, colon carcinoma, familiary adenomatous polyposis carcinoma, and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing's sarcoma, and plasmocytoma.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fuimarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### EXAMPLES

The present disclosure is also described and demonstrated by way of the following examples. However, the use of these and other examples anywhere in the specification is illustrative only and in no way limits the scope and meaning of the disclosure or of any exemplified term. Likewise, the disclosure is not limited to any particular preferred embodiment or aspect described herein. Indeed, many modifications and variations may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. 2-(2,6-Dioxopiperidin-3-yl)-5-((6-oxo-6-(piperidin-1-yl)hexyl)amino)isoindoline-1,3-dione (GS-001)

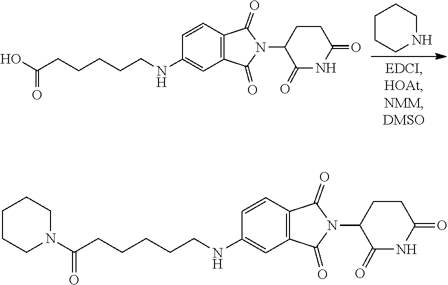

To a mixture of piperidine (22 mg, 258 μmol) and 6-((2-(2,6-dioxopiperidin-3-yl)-1,3-dioxoisoindolin-5-yl)amino)hexanoic acid (50 mg, 129 μmol) in DMSO (1 mL) were added EDCI (40 mg, 258 μmol), HOAT (35 mg, 258 μmol) and NMM (39 mg, 387 μmol). After the reaction was stirred at rt overnight, the mixture was purified by C18 chromatography (0-70% MeCN in H₂O) to provide the title compound (50.5 mg, yield: 86%) as yellow solid. MS (ESI) m/z=455.6 [M+H]⁺.

### Example 2. 5-((6-(4-(4-(8-Bromoquinoxalin-2-yl)-1H-pyrazol-1-yl)piperidin-1-yl)-6-oxohexyl)amino)-2-(2,6-dioxopiperidin-3-yl)isoindoline-1,3-dione (GS-002)

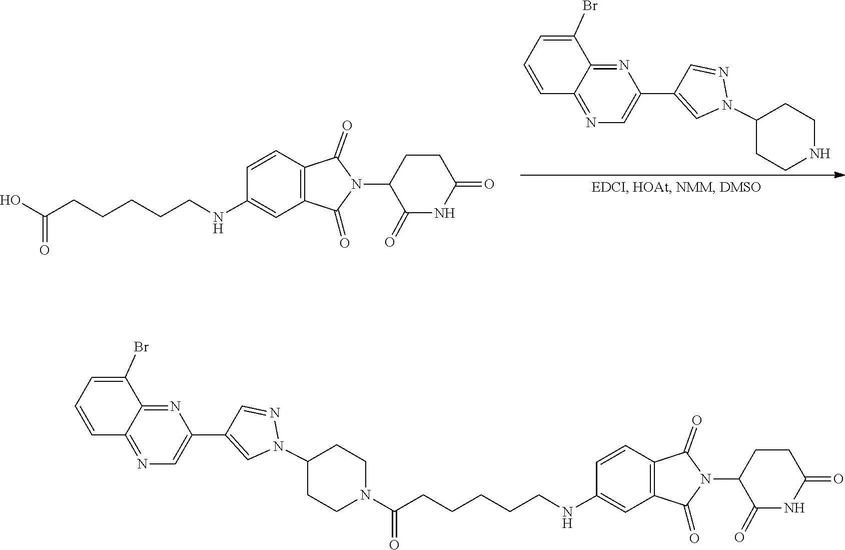 [SMILES: O=C(O)CCCCCNc1ccc2c(c1)C(=O)N(C1CCC(=O)NC1=O)C2=O ; Brc1cccc2ncc(-c3cnn(C4CCNCC4)c3)nc12 ; O=C1CCC(N2C(=O)c3ccc(NCCCCCC(=O)N4CCC(n5cc(-c6cnc7cccc(Br)c7n6)cn5)CC4)cc3C2=O)C(=O)N1]

GS-002 was synthesized following the standard procedure for preparing GS-005 (60 mg, yield: 64%). MS (ESI) m/z=727.4 [M+H]⁺.

### Example 3. 5-((6-(4-(1H-Pyrazol-1-yl)piperidin-1-yl)-6-oxohexyl)amino)-2-(2,6-dioxopiperidin-3-yl)isoindoline-1,3-dione (GS-003)

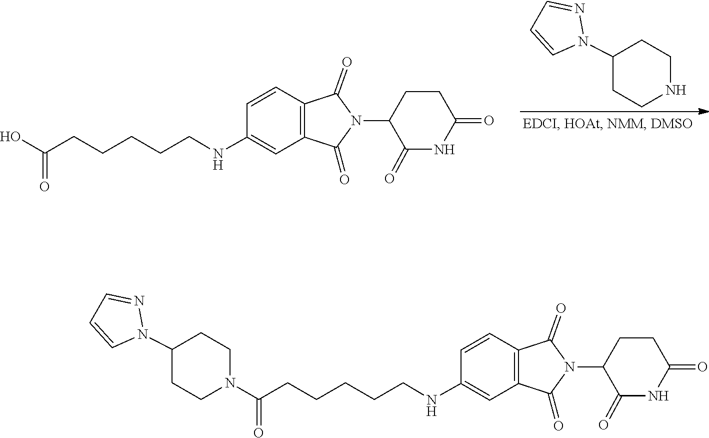 [SMILES: O=C(O)CCCCCNc1ccc2c(c1)C(=O)N(C1CCC(=O)NC1=O)C2=O ; c1cnn(C2CCNCC2)c1 ; O=C1CCC(N2C(=O)c3ccc(NCCCCCC(=O)N4CCC(n5cccn5)CC4)cc3C2=O)C(=O)N1]

GS-003 was synthesized following the standard procedure for preparing GS-001 (44.7 mg, yield: 67%). MS (ESI) m/z=521.6 [M+H]⁺.

### Example 4. 2-(3-(4-(4-(8-Bromoquinoxalin-2-yl)-1H-pyrazol-1-yl)piperidin-1-yl)phenyl)-N-((2-(2,6-dioxopiperidin-3-yl)-1-oxoisoindolin-5-yl)methyl)-2,2-difluoroacetamide (GS-004)

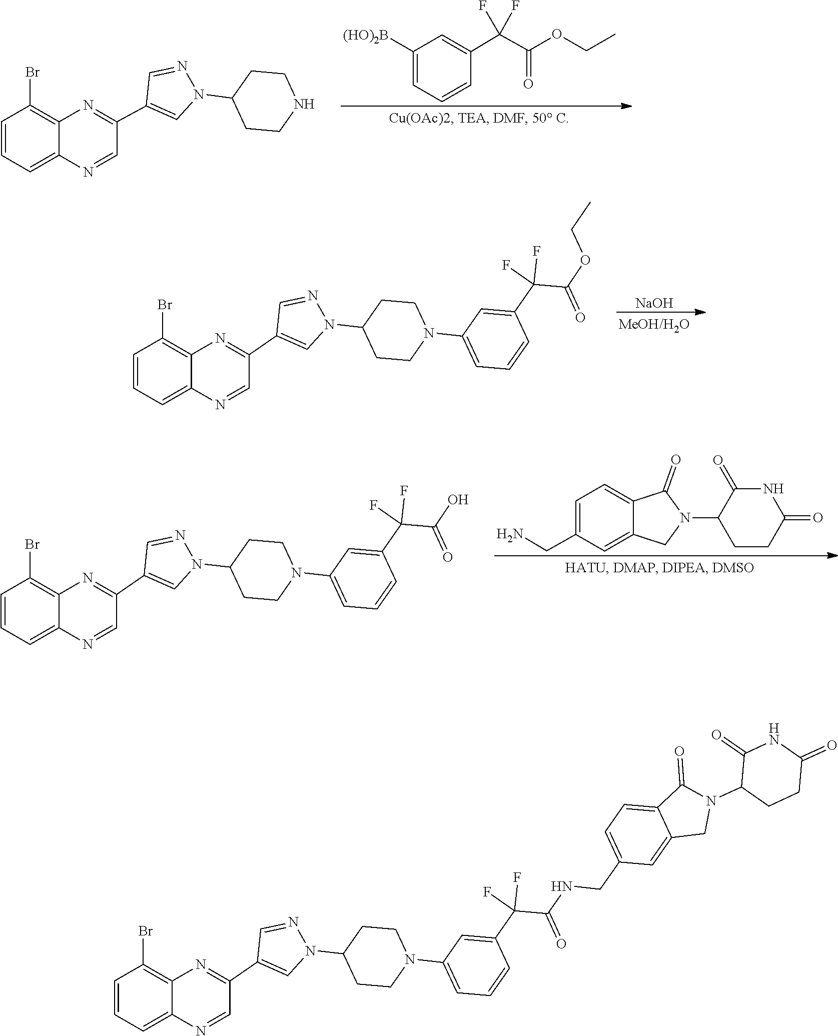

Step 1: Synthesis of ethyl 2-(3-(4-(4-(8-bromoquinoxalin-2-yl)-1H-pyrazol-1-yl)piperidin-1-yl)phenyl)-2,2-difluoroacetate

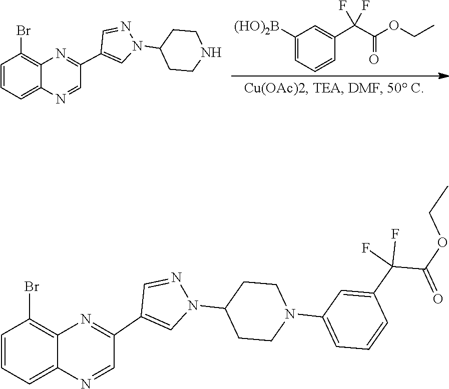 [SMILES: Brc1cccc2ncc(-c3cnn(C4CCNCC4)c3)nc12 ; CCOC(=O)C(F)(F)c1cccc(B(O)O)c1 ; CCOC(=O)C(F)(F)c1cccc(N2CCC(n3cc(-c4cnc5cccc(Br)c5n4)cn3)CC2)c1]

To a mixture of 8-bromo-2-[1-(4-piperidyl)pyrazol-4-yl]quinoxaline (100 mg, 279 μmol) and [3-(2-ethoxy-1,1-difluoro-2-oxo-ethyl)phenyl]boronic acid (136 mg, 558 μmol) in DMF (3 mL) were added Cu(OAc)₂ (101 mg, 558 μmol) and TEA (85 mg, 837.4 μmol). After the reaction was stirred at 50° C. overnight, the mixture was filtered and purified by C18 chromatography (0-70% MeCN in H₂O) to provide the title compound (35 mg, yield: 23%) as white solid. MS (ESI) m/z=556.4 [M+H]⁺.

Step 2: Synthesis of 2-(3-(4-(4-(8-bromoquinoxalin-2-yl)-1H-pyrazol-1-yl)piperidin-1-yl)phenyl)-2,2-difluoroacetic acid

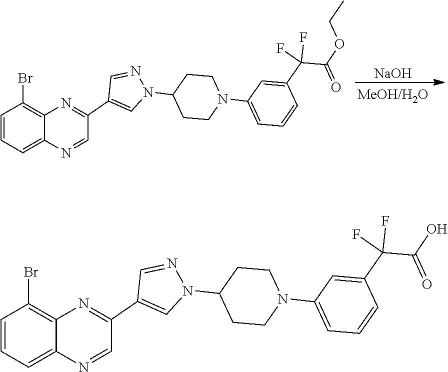 [SMILES: CCOC(=O)C(F)(F)c1cccc(N2CCC(n3cc(-c4cnc5cccc(Br)c5n4)cn3)CC2)c1 ; "NaOH" ; O=C(O)C(F)(F)c1cccc(N2CCC(n3cc(-c4cnc5cccc(Br)c5n4)cn3)CC2)c1]

To a solution of ethyl 2-(3-(4-(4-(8-bromoquinoxalin-2-yl)-1H-pyrazol-1-yl)piperidin-1-yl)phenyl)-2,2-difluoroacetate (35 mg, 63 μmol) in methanol (0.5 mL) and water (0.1 mL) was added NaOH (12 mg, 314 μmol). After the reaction was stirred at rt for 1 h, the mixture was purified by C18 chromatography (0-70% MeCN in H₂O) to provide the title compound (22 mg, yield: 66%) as yellow solid. MS (ESI) m/z=528.4 [M+H]⁺.

Step 3: Synthesis of 2-(3-(4-(4-(8-bromoquinoxalin-2-yl)-1H-pyrazol-1-yl)piperidin-1-yl)phenyl)-N-((2-(2,6-dioxopiperidin-3-yl)-1-oxoisoindolin-5-yl)methyl)-2,2-difluoroacetamide

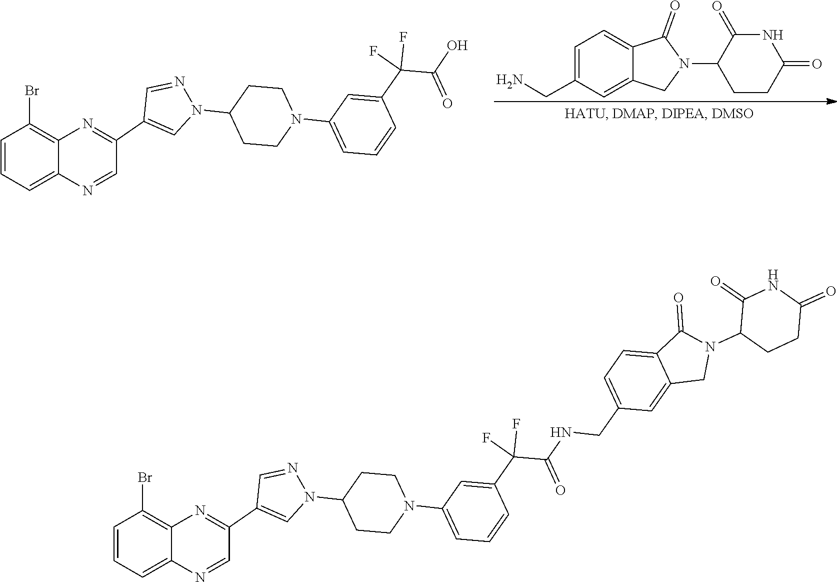 [SMILES: O=C(O)C(F)(F)c1cccc(N2CCC(n3cc(-c4cnc5cccc(Br)c5n4)cn3)CC2)c1 ; NCc1ccc2c(c1)CN(C1CCC(=O)NC1=O)C2=O ; O=C1CCC(N2Cc3cc(CNC(=O)C(F)(F)c4cccc(N5CCC(n6cc(-c7cnc8cccc(Br)c8n7)cn6)CC5)c4)ccc3C2=O)C(=O)N1]

To a mixture of 2-(3-(4-(4-(8-bromoquinoxalin-2-yl)-1H-pyrazol-1-yl)piperidin-1-yl)phenyl)-2,2-difluoroacetic acid (20 mg, 38 μmol) and 3-[5-(aminomethyl)-1-oxo-isoindolin-2-yl]piperidine-2,6-dione (12 mg, 45 μmol) in DMSO (0.5 mL) were added DMAP (462 ug, 3.79 μmol), HATU (29 mg, 76 μmol) and DIPEA (15 mg, 113 μmol). After the reaction was stirred at rt overnight, the reaction solution was purified by C18 chromatography (0-70% MeOH in H₂O) to provide the title compound (4.9 mg, yield: 16%) as off-white solid. MS (ESI) m/z=785.4 [M+H]⁺.

### Example 5. 2-(2,6-Dioxopiperidin-3-yl)-5-((6-oxo-6-(4-(4-(quinoxalin-2-yl)-1H-pyrazol-1-yl)piperidin-1-yl)hexyl)amino)isoindoline-1,3-dione (GS-005)

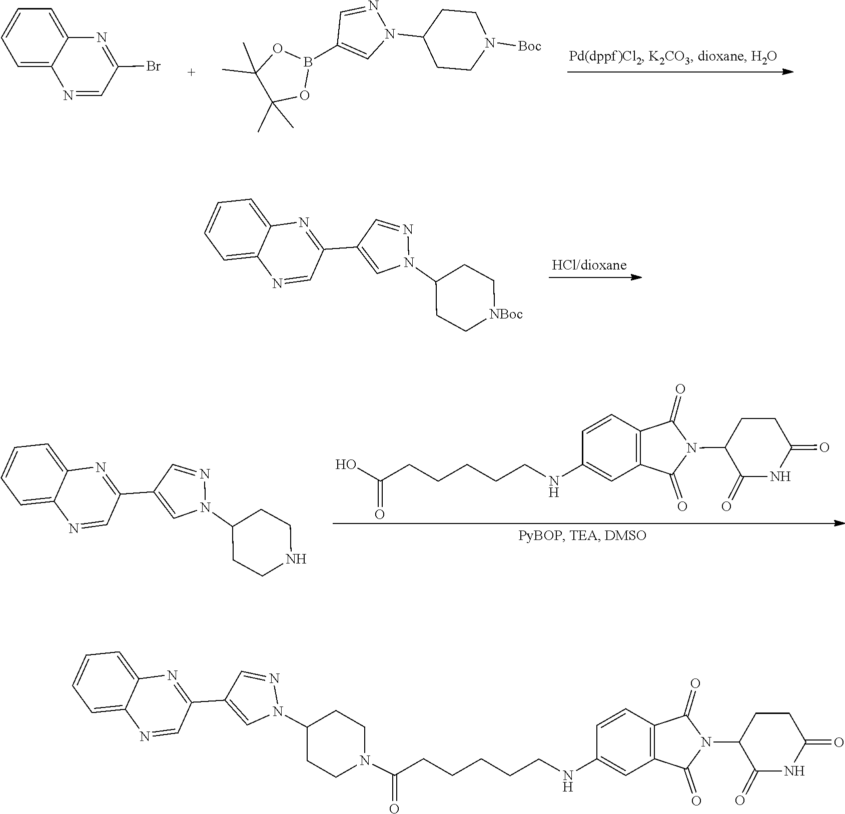 [SMILES: Brc1cnc2ccccc2n1 ; CC(C)(C)OC(=O)N1CCC(n2cc(B3OC(C)(C)C(C)(C)O3)cn2)CC1 ; CC(C)(C)OC(=O)N1CCC(n2cc(-c3cnc4ccccc4n3)cn2)CC1 ; c1ccc2nc(-c3cnn(C4CCNCC4)c3)cnc2c1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(n5cc(-c6cnc7ccccc7n6)cn5)CC4)cc3C2=O)C(=O)N1]

Step 1: Synthesis of tert-butyl 4-(4-(quinoxalin-2-yl)-1H-pyrazol-1-yl)piperidine-1-carboxylate

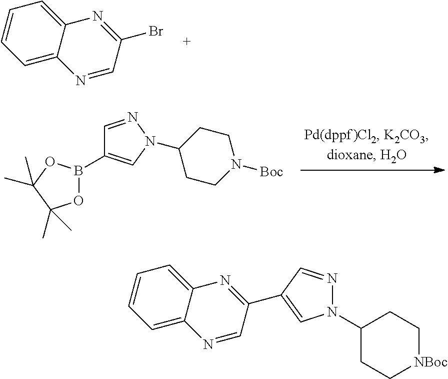 [SMILES: Brc1cnc2ccccc2n1 ; CC(C)(C)OC(=O)N1CCC(n2cc(B3OC(C)(C)C(C)(C)O3)cn2)CC1 ; CC(C)(C)OC(=O)N1CCC(n2cc(-c3cnc4ccccc4n3)cn2)CC1]

A mixture of 2-bromoquinoxaline (100 mg, 478.37 μmol), tert-butyl 4-[4-(4,4,5,5-tetramethyl-1,3,2-dioxaborolan-2-yl)pyrazol-1-yl]piperidine-1-carboxylate (270.72 mg, 717.56 μmol), Pd(dppf)Cl₂ (35.00 mg, 47.84 μmol), K₂CO₃ (132.23 mg, 956.74 μmol) in water (0.1 mL) and dioxane (0.4 mL) was stirred at 80° C. for 2 h. Then, the mixture was concentrated. The resulting residue was purified by silica gel chromatography to provide the title compound (140 mg, yield: 77%). MS (ESI) m/z=380.5 [M+H]⁺.

Step 2: Synthesis of 2-(1-(piperidin-4-yl)-1H-pyrazol-4-yl)quinoxaline

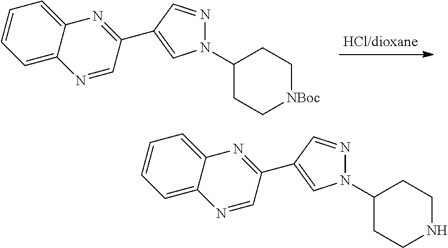

A solution of tert-butyl 4-(4-quinoxalin-2-ylpyrazol-1-yl)piperidine-1-carboxylate (140 mg, 368.95 μmol) in HCl solution (4 M in dioxane, 0.4 mL) and DCM (0.3 mL) was stirred for 2 h. Then, the mixture was concentrated to provide the crude product (95 mg, yield: 82%) which was used directly in the next step without further purification. MS (ESI) m/z=280.3 [M+H]⁺.

Step 3: Synthesis of 2-(2,6-dioxopiperidin-3-yl)-5-((6-oxo-6-(4-(4-(quinoxalin-2-yl)-1H-pyrazol-1-yl)piperidin-1-yl)hexyl)amino)isoindoline-1,3-dione

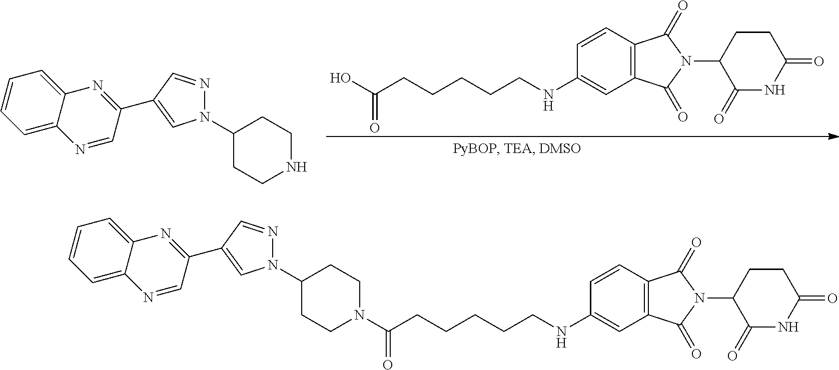

A solution of 2-(1-(piperidin-4-yl)-1H-pyrazol-4-yl)quinoxaline (30 mg, 95.00 μmol, HCl salt), 6-((2-(2,6-dioxopiperidin-3-yl)-1,3-dioxoisoindolin-5-yl)amino)hexanoic acid (33.45 mg, 86.36 μmol), PyBOP (67.41 mg, 129.54 μmol), Et₃N (34.96 mg, 345.44 μmol) in DMSO (0.4 mL) was stirred at rt overnight. Then, the mixture was purified by reverse phase chromatography to provide the title compound (15 mg, yield: 27%). MS (ESI) m/z=649.7 [M+H]⁺.

### Example 6. 2-(2,6-Dioxopiperidin-3-yl)-5-((6-oxo-6-(4-(4-(quinolin-2-yl)-1H-pyrazol-1-yl)piperidin-1-yl)hexyl)amino)isoindoline-1,3-dione (GS-006)

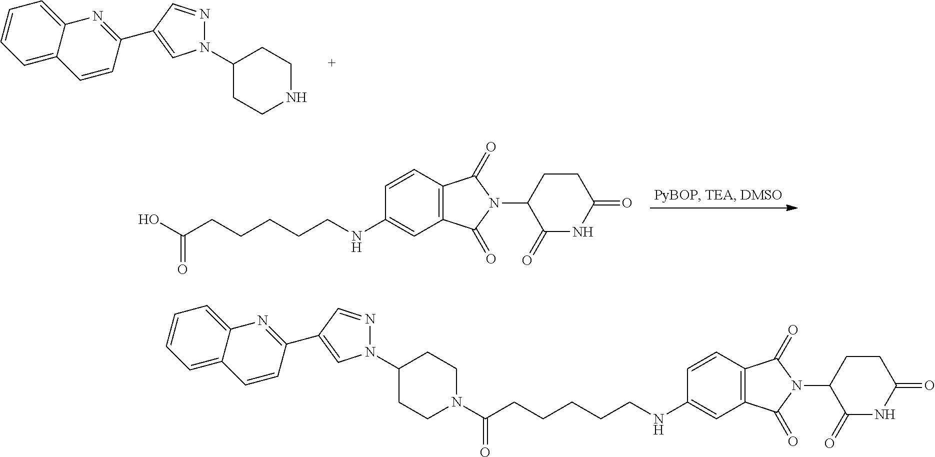

GS-006 was synthesized following the standard procedure for preparing GS-005 (23 mg, yield: 35%). MS (ESI) m/z=648.8 [M+H]⁺.

### Example 7. 5-(4-(4-(8-Bromoquinoxalin-2-yl)-1H-pyrazol-1-yl)piperidin-1-yl)-N-((2-(2,6-dioxopiperidin-3-yl)-1-oxoisoindolin-5-yl)methyl)-2,2-difluoro-5-oxopentanamide (GS-007)

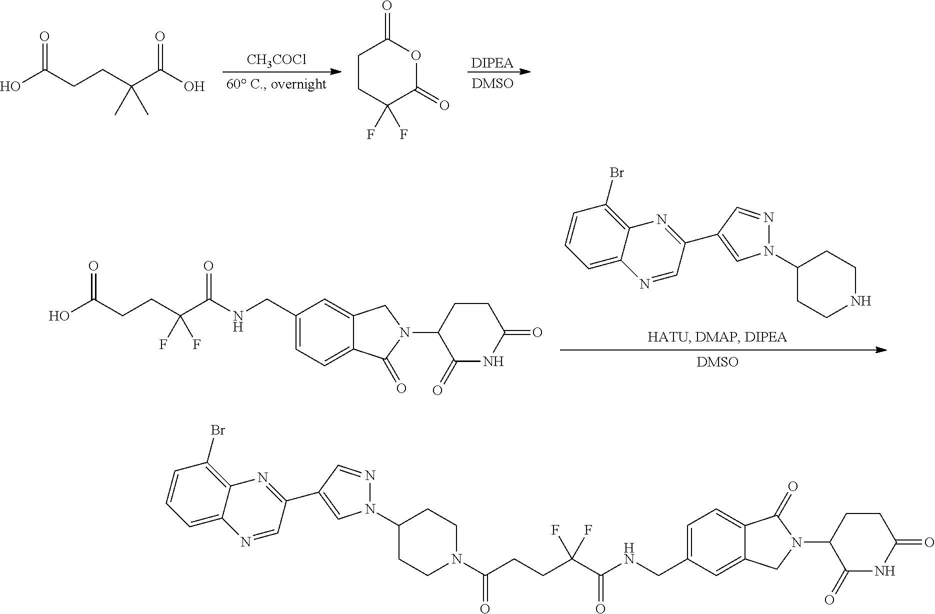

Step 1: Synthesis of 3,3-difluorodihydro-2H-pyran-2,6(3H)-dione

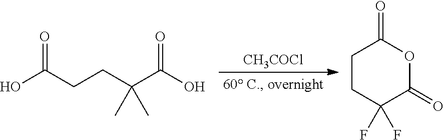

A solution of 2,2-difluoropentanedioic acid (100 mg, 595 μmol) in acetyl chloride (2 mL) was stirred at 60° C. overnight. The reaction mixture was concentrated, and the resulting residue was used directly in the next step without further purification.

Step 2: Synthesis of 5-(((2-(2,6-dioxopiperidin-3-yl)-1-oxoisoindolin-5-yl)methyl)amino)-4,4-difluoro-5-oxopentanoic acid

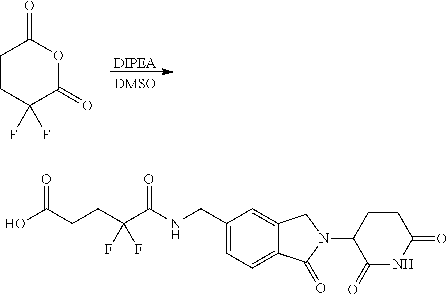 [SMILES: O=C1CCC(F)(F)C(=O)O1 ; "DIPEA" ; O=C(O)CCC(F)(F)C(=O)NCc1ccc2c(c1)CN(C1CCC(=O)NC1=O)C2=O]

A solution of 3-[5-(aminomethyl)-1-oxo-isoindolin-2-yl]piperidine-2,6-dione (100 mg, 1.2 mmol) in DMSO (1 mL) was added 3,3-difluorodihydro-2H-pyran-2,6(3H)-dione (90 mg crude). The resulting mixture was stirred at 60° C. for 1 h. Then it was purified by C18 chromatography (0-70% MeCN in H₂O) to provide the title compound (20 mg, yield: 8%) as yellow solid. MS (ESI) m/z=424.3 [M+H]⁺.

Step 3: Synthesis of 5-(4-(4-(8-bromoquinoxalin-2-yl)-1H-pyrazol-1-yl)piperidin-1-yl)-N-((2-(2,6-dioxopiperidin-3-yl)-1-oxoisoindolin-5-yl)methyl)-2,2-difluoro-5-oxopentanamide

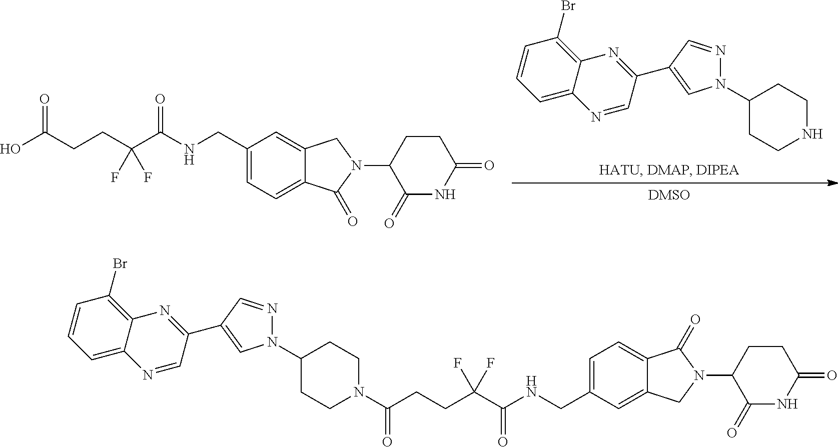 [SMILES: O=C(O)CCC(F)(F)C(=O)NCc1ccc2c(c1)CN(C1CCC(=O)NC1=O)C2=O ; Brc1cccc2ncc(-c3cnn(C4CCNCC4)c3)nc12 ; O=C1CCC(N2Cc3cc(CNC(=O)C(F)(F)CCC(=O)N4CCC(n5cc(-c6cnc7cccc(Br)c7n6)cn5)CC4)ccc3C2=O)C(=O)N1]

To a mixture of 5-(((2-(2,6-dioxopiperidin-3-yl)-1-oxoisoindolin-5-yl)methyl)amino)-4,4-difluoro-5-oxopentanoic acid (10 mg, 24 μmol) and 8-bromo-2-[1-(4-piperidyl)pyrazol-4-yl]quinoxaline (13 mg, 35 μmol) in DMSO (0.5 mL) were added HATU (18 mg, 47 μmol), DMAP (288 ug, 2 μmol) and DIPEA (12 mg, 94 μmol). The reaction mixture was stirred at rt overnight, before it was purified by C18 chromatography (0-70% MeOH in H₂O) to provide the title compound (2.3 mg, yield: 13%) as off-white solid. MS (ESI) m/z=765.5 [M+H]⁺.

### Example 8. 2-(2,6-Dioxopiperidin-3-yl)-5-((6-oxo-6-(4-(4-(1,2,3,4-tetrahydroquinoxalin-2-yl)-1H-pyrazol-1-yl)piperidin-1-yl)hexyl)amino)isoindoline-1,3-dione (GS-008)

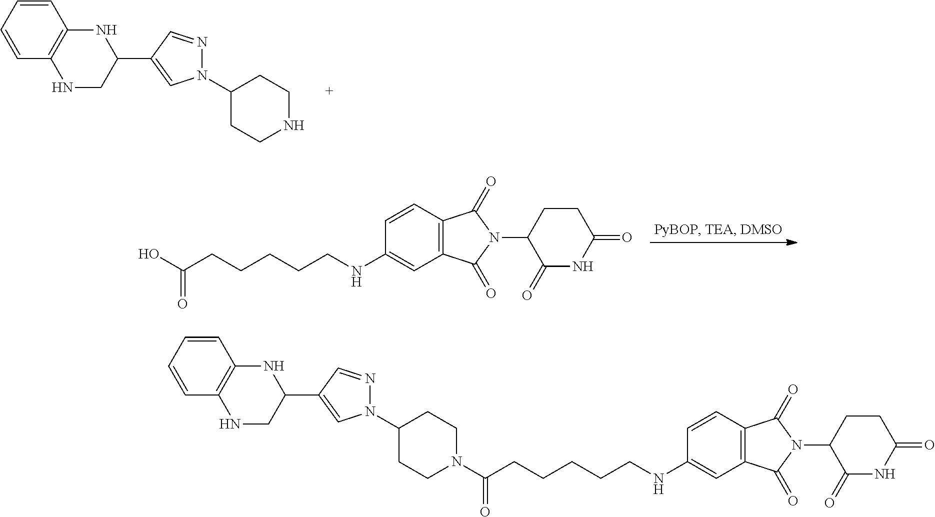

GS-008 was synthesized following the standard procedure for preparing GS-005 (3.8 mg, yield: 7%). MS (ESI) m/z=653.6 [M+H]⁺.

### Example 9. 2-(2,6-Dioxopiperidin-3-yl)-5-((6-oxo-6-(4-(4-(pyrazin-2-yl)-1H-pyrazol-1-yl)piperidin-1-yl)hexyl)amino)isoindoline-1,3-dione (GS-009)

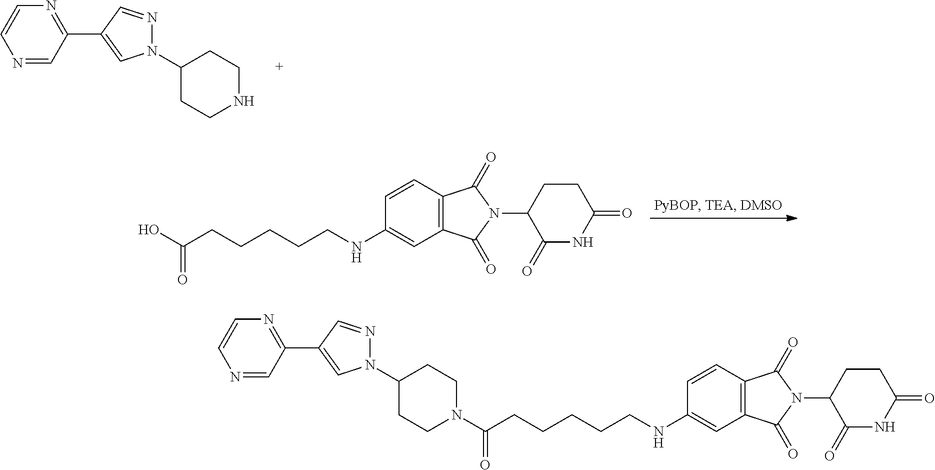 [SMILES: c1cnc(-c2cnn(C3CCNCC3)c2)cn1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(n5cc(-c6cnccn6)cn5)CC4)cc3C2=O)C(=O)N1]

GS-009 was synthesized following the standard procedure for preparing GS-005 (3.7 mg, yield: 6%). MS (ESI) m/z=599.6 [M+H]⁺.

### Example 10. 2-(2,6-Dioxopiperidin-3-yl)-5-((6-oxo-6-(4-phenylpiperidin-1-yl)hexyl)amino)isoindoline-1,3-dione (GS-010)

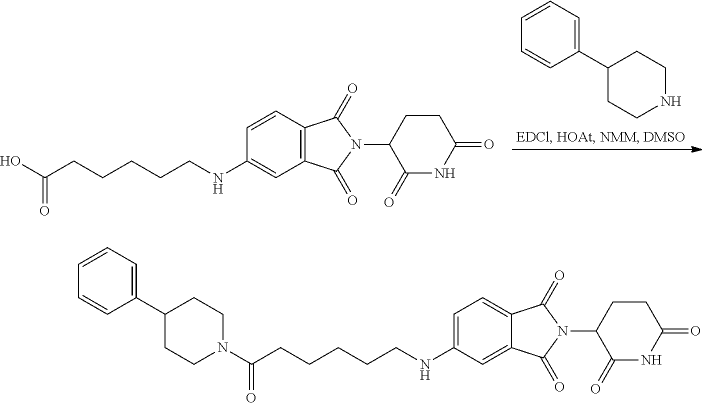 [SMILES: O=C(O)CCCCCNc1ccc2c(c1)C(=O)N(C1CCC(=O)NC1=O)C2=O ; c1ccc(C2CCNCC2)cc1 ; O=C1CCC(N2C(=O)c3ccc(NCCCCCC(=O)N4CCC(c5ccccc5)CC4)cc3C2=O)C(=O)N1]

GS-010 was synthesized following the standard procedure for preparing GS-001 (82 mg, yield: 61.6%). MS (ESI) m/z=531.5 [M+H]⁺.

### Example 11. 2-(2,6-Dioxopiperidin-3-yl)-5-((6-(4-morpholinopiperidin-1-yl)-6-oxohexyl)amino)isoindoline-1,3-dione (GS-011)

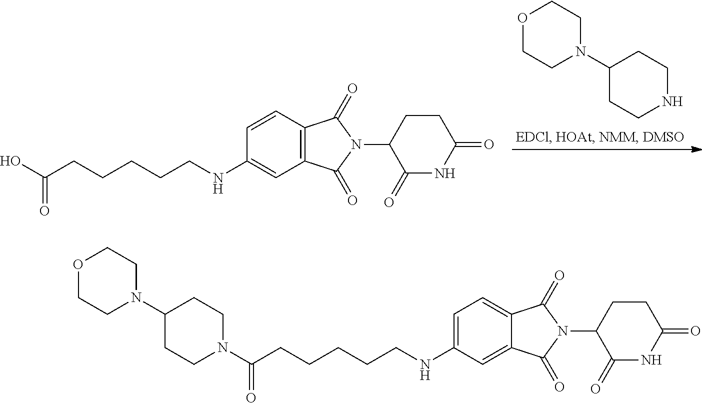 [SMILES: O=C(O)CCCCCNc1ccc2c(c1)C(=O)N(C1CCC(=O)NC1=O)C2=O ; C1CC(N2CCOCC2)CCN1 ; O=C1CCC(N2C(=O)c3ccc(NCCCCCC(=O)N4CCC(N5CCOCC5)CC4)cc3C2=O)C(=O)N1]

GS-011 was synthesized following the standard procedure for preparing GS-001 (112 mg, yield: 83%). MS (ESI) m/z=540.6 [M+H]⁺.

### Example 12. 5-((6-([1,4′-Bipiperidin]-1′-yl)-6-oxohexyl)amino)-2-(2,6-dioxopiperidin-3-yl)isoindoline-1,3-dione (GS-012)

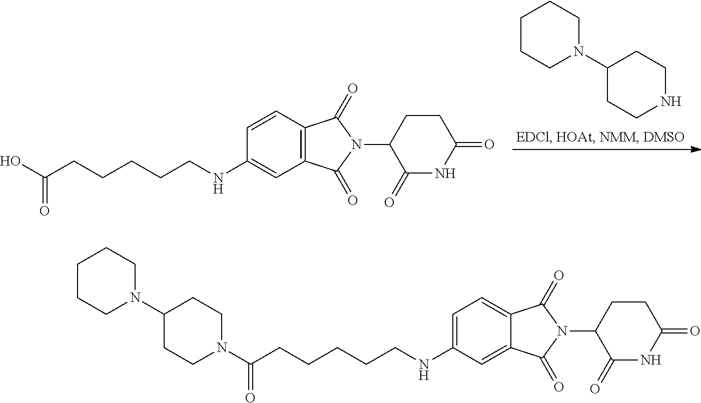 [SMILES: O=C(O)CCCCCNc1ccc2c(c1)C(=O)N(C1CCC(=O)NC1=O)C2=O ; C1CCN(C2CCNCC2)CC1 ; O=C1CCC(N2C(=O)c3ccc(NCCCCCC(=O)N4CCC(N5CCCCC5)CC4)cc3C2=O)C(=O)N1]

GS-012 was synthesized following the standard procedure for preparing GS-001 (95 mg, yield: 71%). MS (ESI) m/z=538.7 [M+H]⁺.

### Example 13. 2-(2,6-Dioxopiperidin-3-yl)-5-((6-(4-(4-methylpiperazin-1-yl)piperidin-1-yl)-6-oxohexyl)amino)isoindoline-1,3-dione (GS-013)

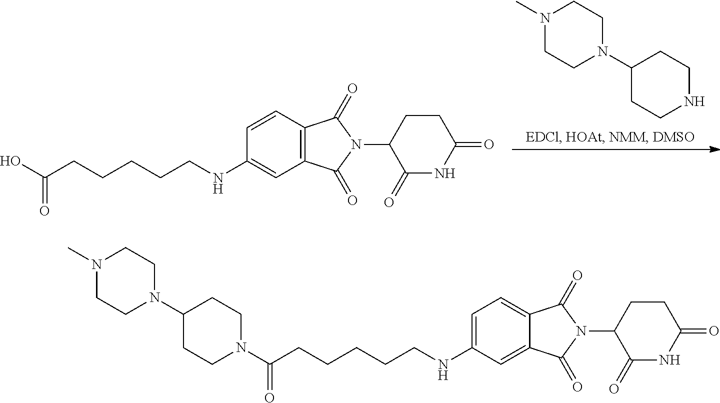

GS-013 was synthesized following the standard procedure for preparing GS-001 (111 mg, yield: 80.6%). MS (ESI) m/z=553.6 [M+H]⁺.

### Example 14. 2-(2,6-Dioxopiperidin-3-yl)-5-((6-oxo-6-(4-(pyrimidin-2-yl)piperidin-1-yl)hexyl)amino)isoindoline-1,3-dione (GS-014)

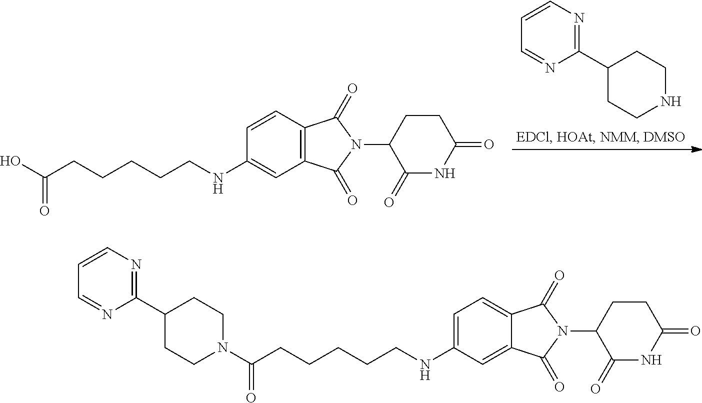 [SMILES: O=C(O)CCCCCNc1ccc2c(c1)C(=O)N(C1CCC(=O)NC1=O)C2=O ; c1cnc(C2CCNCC2)nc1 ; O=C1CCC(N2C(=O)c3ccc(NCCCCCC(=O)N4CCC(c5ncccn5)CC4)cc3C2=O)C(=O)N1]

GS-014 was synthesized following the standard procedure for preparing GS-001 (30.7 mg, yield: 31.3%). MS (ESI) m/z=533.6 [M+H]⁺.

### Example 15. 2-(2,6-Dioxopiperidin-3-yl)-5-((6-oxo-6-(4-(3-(quinoxalin-2-yl)pyrrolidin-1-yl)piperidin-1-yl)hexyl)amino)isoindoline-1,3-dione (GS-015)

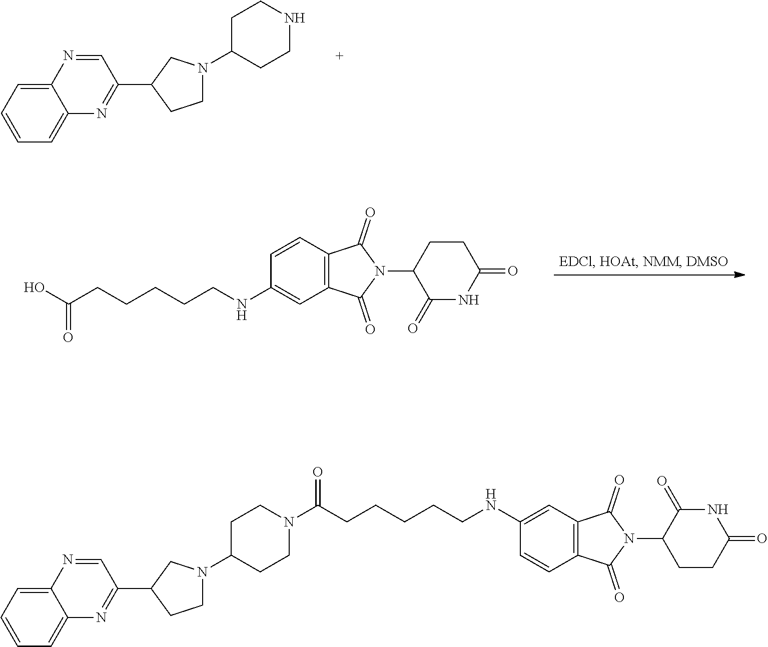 [SMILES: c1ccc2nc(C3CCN(C4CCNCC4)C3)cnc2c1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(N5CCC(c6cnc7ccccc7n6)C5)CC4)cc3C2=O)C(=O)N1]

GS-015 was synthesized following the standard procedure for preparing GS-005 (17 mg, yield: 45.8%). MS (ESI) m/z=652.8 [M+H]⁺.

### Example 16. 2-(2,6-Dioxopiperidin-3-yl)-5-((6-(4-(2-methylbenzoyl)piperazin-1-yl)-6-oxohexyl)amino)isoindoline-1,3-dione (GS-016)

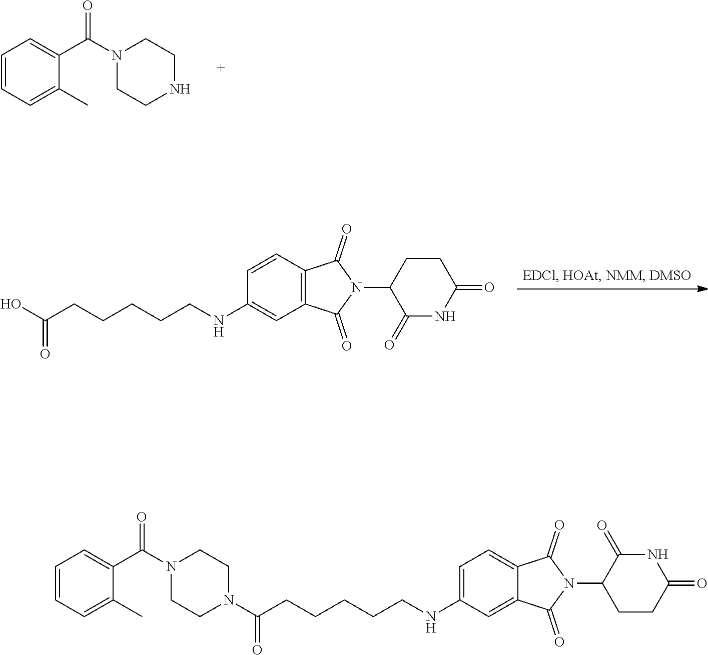 [SMILES: Cc1ccccc1C(=O)N1CCNCC1 ; O=C(O)CCCCCNc1ccc2c(c1)C(=O)N(C1CCC(=O)NC1=O)C2=O ; Cc1ccccc1C(=O)N1CCN(C(=O)CCCCCNc2ccc3c(c2)C(=O)N(C2CCC(=O)NC2=O)C3=O)CC1]

GS-016 was synthesized following the standard procedure for preparing GS-001 (50.2 mg, yield: 58.4%). MS (ESI) m/z=574.6 [M+H]⁺.

### Example 17. 2-(2,6-Dioxopiperidin-3-yl)-5-((6-oxo-6-(4-(pyridin-2-yl)piperidin-1-yl)hexyl)amino)isoindoline-1,3-dione (GS-017)

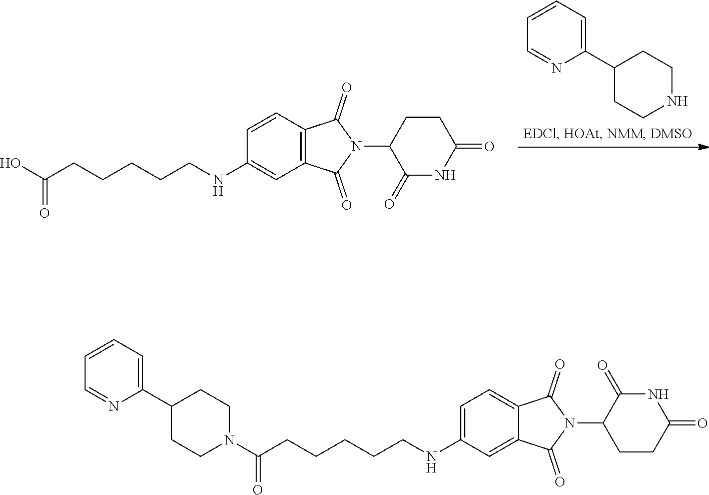 [SMILES: O=C(O)CCCCCNc1ccc2c(c1)C(=O)N(C1CCC(=O)NC1=O)C2=O ; c1ccc(C2CCNCC2)nc1 ; O=C1CCC(N2C(=O)c3ccc(NCCCCCC(=O)N4CCC(c5ccccn5)CC4)cc3C2=O)C(=O)N1]

GS-017 was synthesized following the standard procedure for preparing GS-001 (40.7 mg, yield: 48.1%). MS (ESI) m/z=532.7 [M+H]⁺.

### Example 18. 3-(5-(2-(3-(4-(4-(8-Bromoquinoxalin-2-yl)-1H-pyrazol-1-yl)piperidin-1-yl)-3-oxopropoxy)ethoxy)-1-oxoisoindolin-2-yl)piperidine-2,6-dione (GS-018)

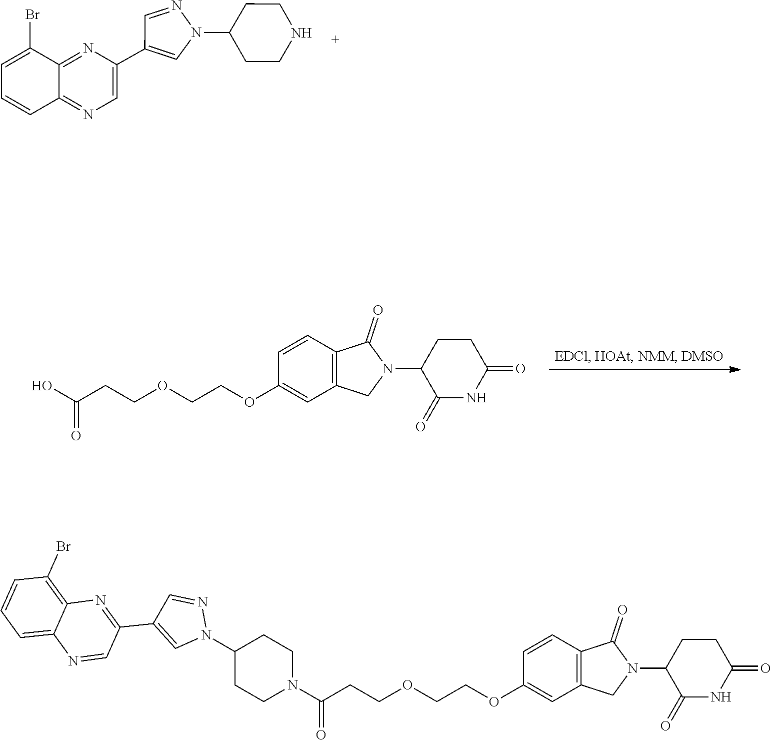 [SMILES: Brc1cccc2ncc(-c3cnn(C4CCNCC4)c3)nc12 ; O=C(O)CCOCCOc1ccc2c(c1)CN(C1CCC(=O)NC1=O)C2=O ; O=C1CCC(N2Cc3cc(OCCOCCC(=O)N4CCC(n5cc(-c6cnc7cccc(Br)c7n6)cn5)CC4)ccc3C2=O)C(=O)N1]

GS-018 was synthesized following the standard procedure for preparing GS-001 (9.6 mg, yield: 64%). MS (ESI) m/z=716.2 [M+H]⁺.

### Example 19. 2-(2,6-Dioxopiperidin-3-yl)-5-((6-oxo-6-(4-(pyridin-2-yl)piperazin-1-yl)hexyl)amino)isoindoline-1,3-dione (GS-019)

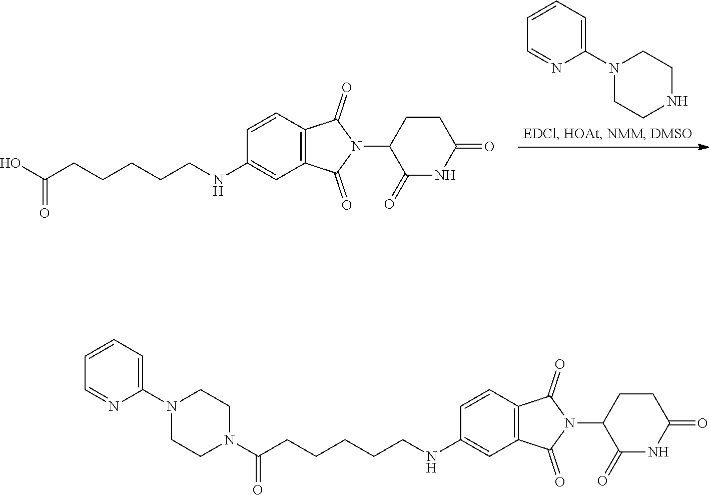 [SMILES: O=C(O)CCCCCNc1ccc2c(c1)C(=O)N(C1CCC(=O)NC1=O)C2=O ; c1ccc(N2CCNCC2)nc1 ; O=C1CCC(N2C(=O)c3ccc(NCCCCCC(=O)N4CCN(c5ccccn5)CC4)cc3C2=O)C(=O)N1]

GA-019 was synthesized following the standard procedure for preparing GS-001 (5.8 mg, yield: 84%). MS (ESI) m/z=533.3 [M+H]⁺.

### Example 20. 6-(4-(4-(8-Bromoquinoxalin-2-yl)-1H-pyrazol-1-yl)piperidin-1-yl)-N-(2-(2,6-dioxopiperidin-3-yl)-1-oxoisoindolin-5-yl)-6-oxohexanamide (GS-020)

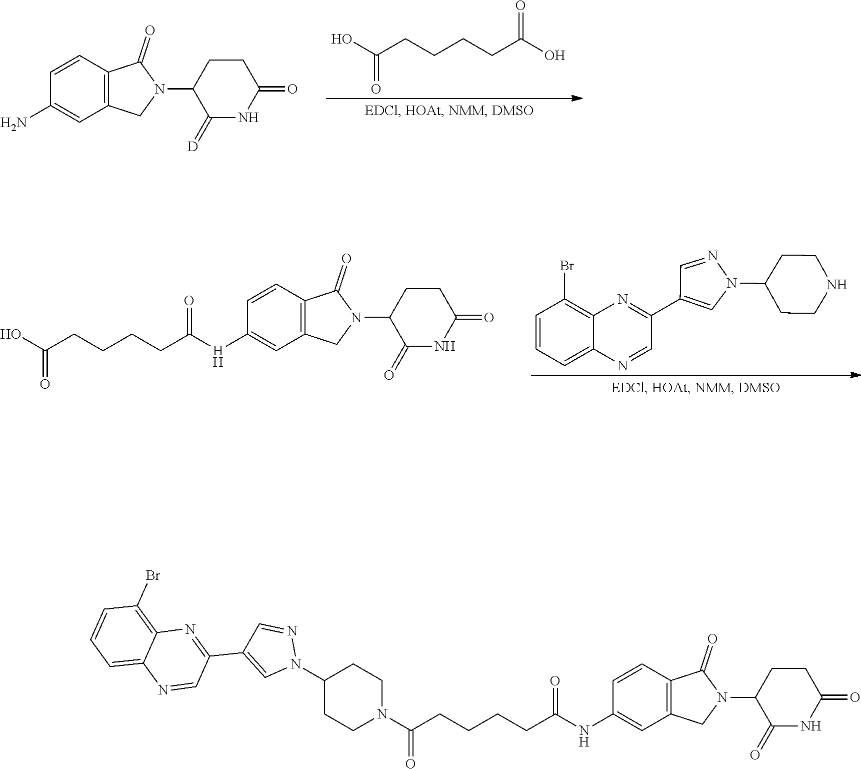

Step 1: Synthesis of 6-((2-(2,6-dioxopiperidin-3-yl)-1-oxoisoindolin-5-yl)amino)-6-oxohexanoic acid

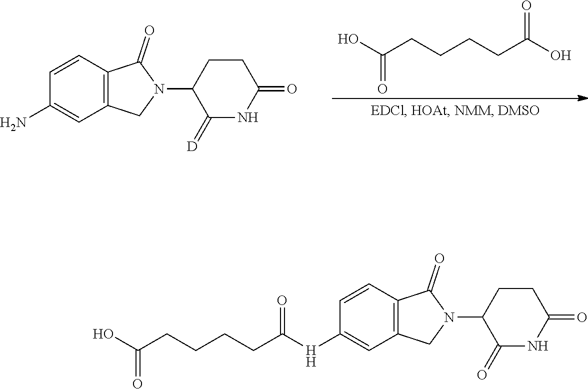

A mixture of 3-(5-amino-1-oxoisoindolin-2-yl)piperidine-2,6-dione (20 mg, 0.07 mmol), adipic acid (33 mg, 0.23 mmol), EDCI (22 mg, 0.12 mmol), HOAT (16 mg, 0.12 mmol) and NMM (21 mg, 021 mmol) in DMSO (2 mL) was stirred at rt for 16 h. The resulting mixture was purified by reverse-phase chromatography to provide the desired product (12 mg, 45% yield) as white solid. MS (ESI) m/z=388.3 [M+H]⁺.

Step 2: Synthesis of 6-(4-(4-(8-bromoquinoxalin-2-yl)-1H-pyrazol-1-yl)piperidin-1-yl)-N-(2-(2,6-dioxopiperidin-3-yl)-1-oxoisoindolin-5-yl)-6-oxohexanamide

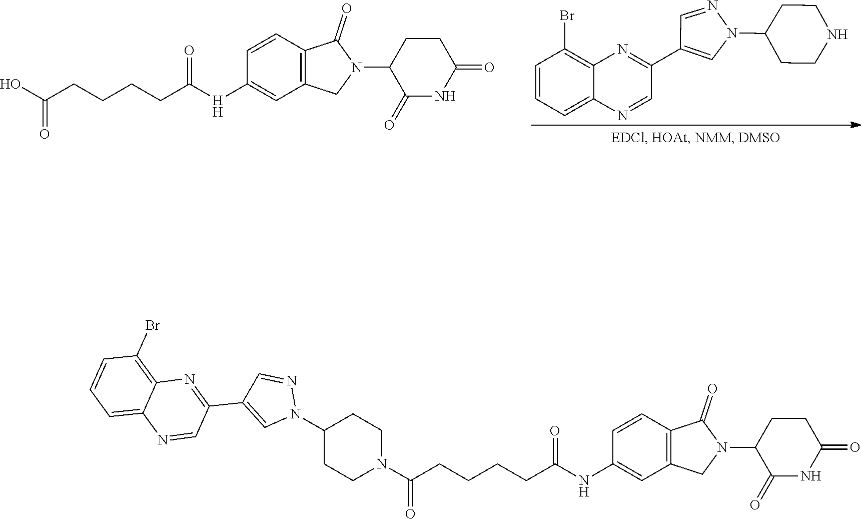

GS-020 was synthesized following the standard procedure for preparing GS-001 (8.7 mg, yield: 41%). MS (ESI) m/z=727.4 [M+H]⁺.

### Example 21. 5-(4-(4-(8-Bromoquinoxalin-2-yl)-1H-pyrazol-1-yl)piperidin-1-yl)-N-((2-(2,6-dioxopiperidin-3-yl)-1-oxoisoindolin-5-yl)methyl)-5-oxopentanamide (GS-021)

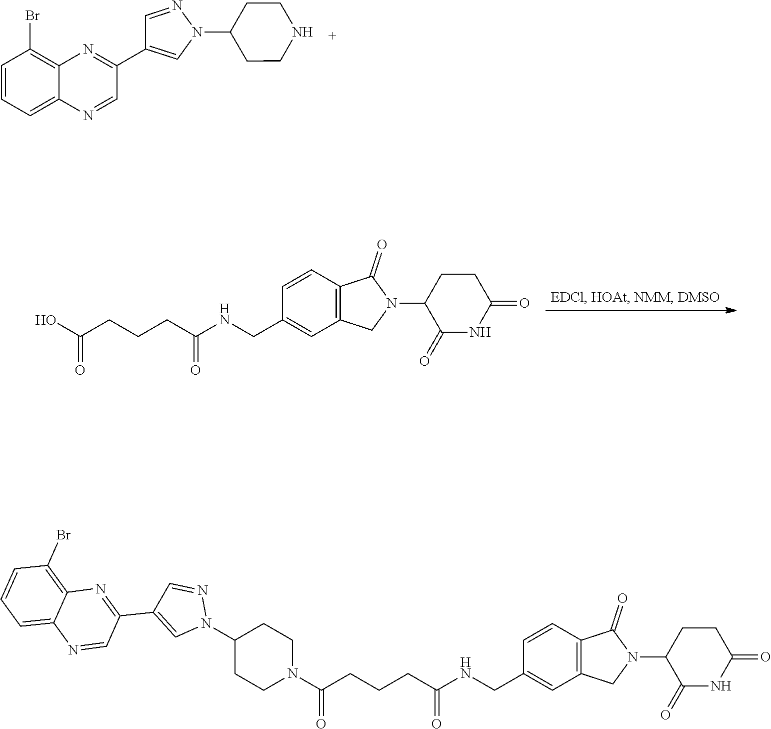 [SMILES: Brc1cccc2ncc(-c3cnn(C4CCNCC4)c3)nc12 ; O=C(O)CCCC(=O)NCc1ccc2c(c1)CN(C1CCC(=O)NC1=O)C2=O ; O=C(CCCC(=O)N1CCC(n2cc(-c3cnc4cccc(Br)c4n3)cn2)CC1)NCc1ccc2c(c1)CN(C1CCC(=O)NC1=O)C2=O]

GS-021 was synthesized following the standard procedure for preparing GS-001 (6.1 mg, yield: 43%). MS (ESI) m/z=727.4 [M+H]⁺.

### Example 22. N-(3-(4-(4-(8-bromoquinoxalin-2-yl)-1H-pyrazol-1-yl)piperidin-1-yl)-3-oxopropyl)-2-((2-(2,6-dioxopiperidin-3-yl)-1-oxoisoindolin-5-yl)oxy)acetamide (GS-022)

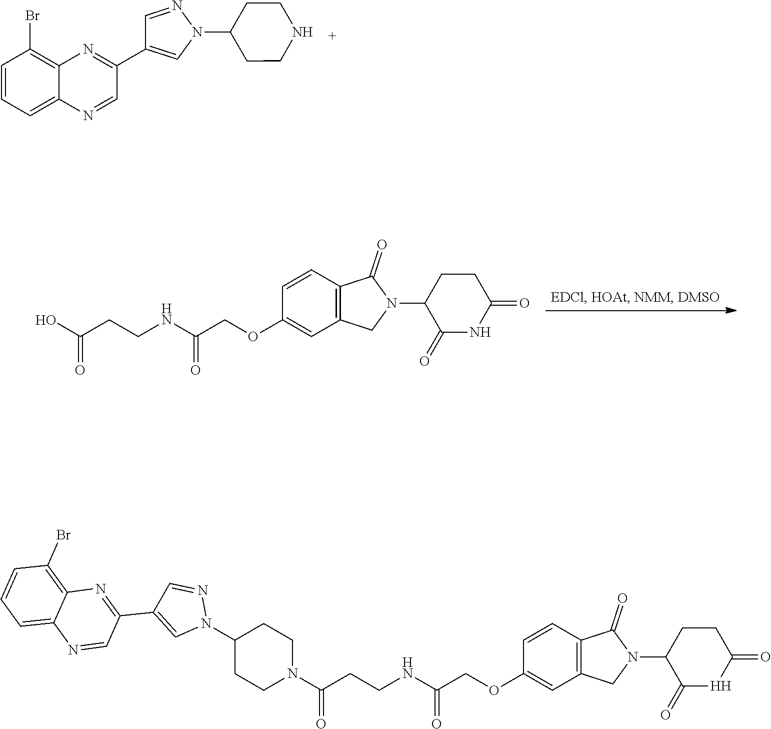

GS-022 was synthesized following the standard procedure for preparing GS-001 (3 mg, yield: 20%). MS (ESI) m/z=729.3 [M+H]⁺.

### Example 23. 3-(4-((2-((3-((4-(4-(8-Bromoquinoxalin-2-yl)-1H-pyrazol-1-yl)piperidin-1-yl)methyl)phenyl)amino)pyrimidin-4-yl)amino)-1-oxoisoindolin-2-yl)piperidine-2,6-dione (GS-028)

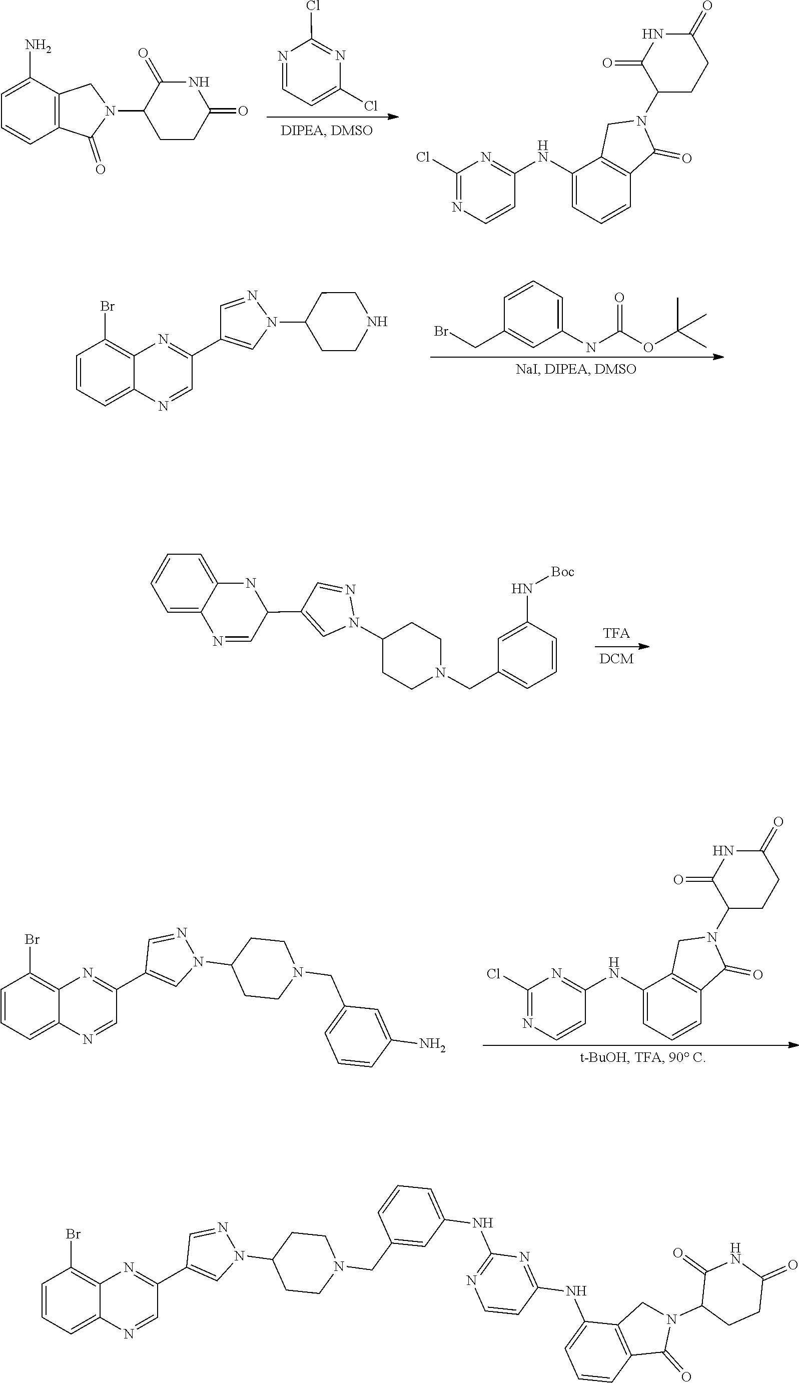

Step 1: Synthesis of 3-(4-((2-chloropyrimidin-4-yl)amino)-1-oxoisoindolin-2-yl)piperidine-2,6-dione

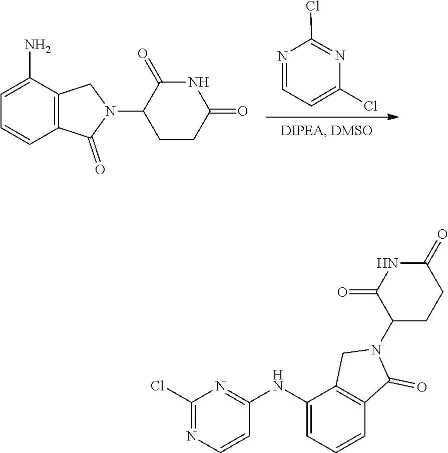

To a solution of 3-(4-amino-1-oxo-isoindolin-2-yl)piperidine-2,6-dione (2.0 g, 7.71 mmol) and 2,4-dichloropyrimidine (2.30 g, 15.42 mmol) in DMF (50 mL) was added DIPEA (5.98 g, 46.26 mmol) at rt. The reaction mixture was stirred at 120° C. for 16 h. The reaction mixture was concentrated and purified by silica gel chromatography (DCM/MeOH=20/1) to provide the title compound (1.5 g, 52% yield). MS (ESI) m/z=372.3 [M+H]⁺.

Step 2: Synthesis of tert-butyl (3-((4-(4-(8-bromoquinoxalin-2-yl)-1H-pyrazol-1-yl)piperidin-1-yl)methyl)phenyl)carbamate

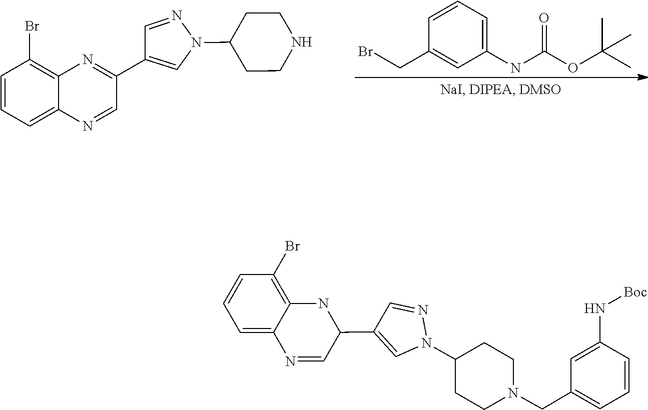

To a solution of 8-bromo-2-(1-(piperidin-4-yl)-1H-pyrazol-4-yl)quinoxaline (130 mg, 275.27 μmol) in DMSO (5 mL) were added NaI (41.26 mg, 275.27 μmol) and DIPEA (106.73 mg, 825.82 μmol) at rt. After the mixture was stirred at rt for 16 h, it was quenched with brine (20 mL), extracted with EtOAc (20 mL×2), and concentrated. The resulting residue was purified by silica gel chromatography (DCM/MeOH=20/1) to provide the title compound (150 mg, 96.7% yield). MS (ESI) m/z=565.4 [M+H]⁺.

Step 3: Synthesis of 3-((4-(4-(8-bromoquinoxalin-2-yl)-1H-pyrazol-1-yl)piperidin-1-yl)methyl)aniline

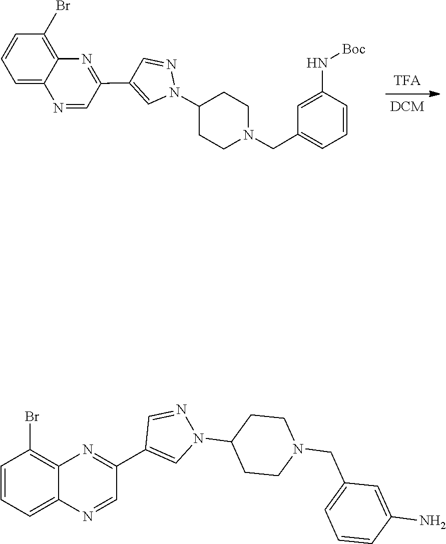 [SMILES: CC(C)(C)OC(=O)Nc1cccc(CN2CCC(n3cc(-c4cnc5cccc(Br)c5n4)cn3)CC2)c1 ; Nc1cccc(CN2CCC(n3cc(-c4cnc5cccc(Br)c5n4)cn3)CC2)c1]

To a solution of tert-butyl (3-((4-(4-(8-bromoquinoxalin-2-yl)-1H-pyrazol-1-yl)piperidin-1-yl)methyl)phenyl)carbamate (100 mg, 177.47 μmol) in DCM (10 mL) was added TFA (2 mL) at rt. After the mixture was stirred at rt for 2 h, it was concentrated to provide the title compound (120 mg, 97.8% yield). MS (ESI) m/z=465.2 [M+H]⁺.

Step 4: Synthesis of 3-(4-((2-((3-((4-(4-(8-bromoquinoxalin-2-yl)-1H-pyrazol-1-yl)piperidin-1-yl)methyl)phenyl)amino)pyrimidin-4-yl)amino)-1-oxoisoindolin-2-yl)piperidine-2,6-dione

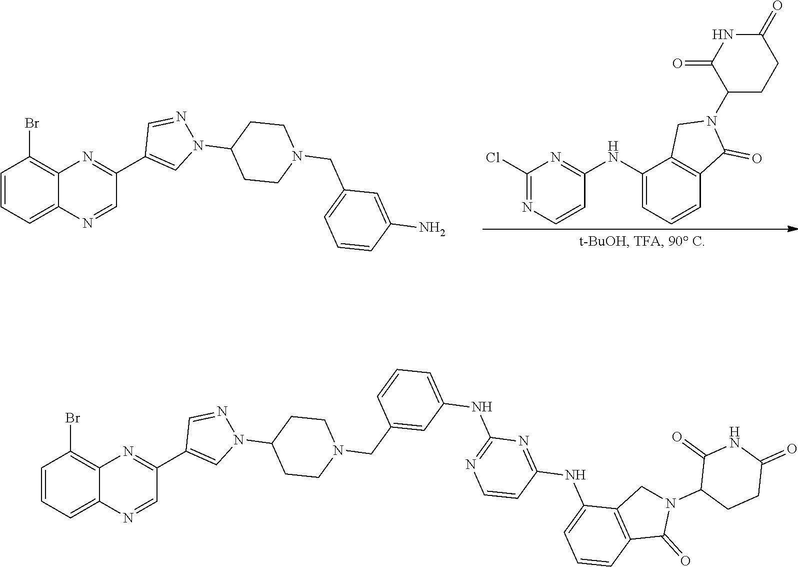 [SMILES: Nc1cccc(CN2CCC(n3cc(-c4cnc5cccc(Br)c5n4)cn3)CC2)c1 ; O=C1CCC(N2Cc3c(Nc4ccnc(Cl)n4)cccc3C2=O)C(=O)N1 ; O=C1CCC(N2Cc3c(Nc4ccnc(Nc5cccc(CN6CCC(n7cc(-c8cnc9cccc(Br)c9n8)cn7)CC6)c5)n4)cccc3C2=O)C(=O)N1]

To a solution of 3-((4-(4-(8-bromoquinoxalin-2-yl)-1H-pyrazol-1-yl)piperidin-1-yl)methyl)aniline (80.00 mg, 172.65 μmol) and 3-(4-((2-chloropyrimidin-4-yl)amino)-1-oxoisoindolin-2-yl)piperidine-2,6-dione (77.02 mg, 207.18 μmol) in tBuOH (5 mL) and DMSO (1 mL) was added TFA (0.5 mL) at rt. After the mixture was stirred at 100° C. for 3 h, it was purified by prep-HPLC to provide a crude product which was further purified by prep-TLC (DCM/MeOH=10/1) to provide the title compound (102 mg, 74% yield). MS (ESI) m/z=800.5 [M+H]⁺.

### Example 24. 3-(4-((2-((4-((4-(4-(8-Bromoquinoxalin-2-yl)-1H-pyrazol-1-yl)piperidin-1-yl)methyl)phenyl)amino)pyrimidin-4-yl)amino)-1-oxoisoindolin-2-yl)piperidine-2,6-dione (GS-030)

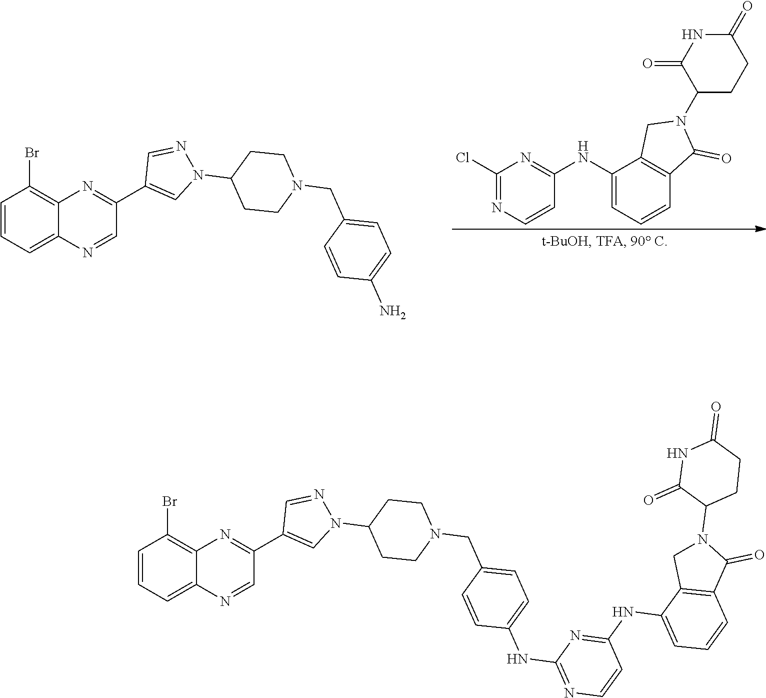 [SMILES: Nc1ccc(CN2CCC(n3cc(-c4cnc5cccc(Br)c5n4)cn3)CC2)cc1 ; O=C1CCC(N2Cc3c(Nc4ccnc(Cl)n4)cccc3C2=O)C(=O)N1 ; O=C1CCC(N2Cc3c(Nc4ccnc(Nc5ccc(CN6CCC(n7cc(-c8cnc9cccc(Br)c9n8)cn7)CC6)cc5)n4)cccc3C2=O)C(=O)N1]

GS-030 was synthesized following the similar procedure for preparing GS-028 (70 mg, yield: 51%). MS (ESI) m/z=800.5 [M+H]⁺.

### Example 25. 3-(1-Oxo-4-((2-((3-((4-(4-(quinoxalin-2-yl)-1H-pyrazol-1-yl)piperidin-1-yl)methyl)phenyl)amino)pyrimidin-4-yl)amino)isoindolin-2-yl)piperidine-2,6-dione (GS-029)

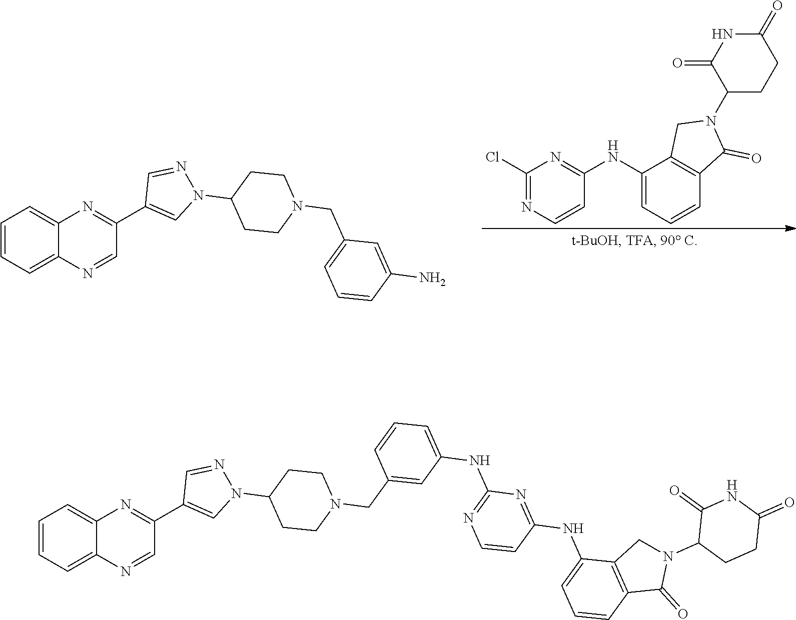 [SMILES: Nc1cccc(CN2CCC(n3cc(-c4cnc5ccccc5n4)cn3)CC2)c1 ; O=C1CCC(N2Cc3c(Nc4ccnc(Cl)n4)cccc3C2=O)C(=O)N1 ; O=C1CCC(N2Cc3c(Nc4ccnc(Nc5cccc(CN6CCC(n7cc(-c8cnc9ccccc9n8)cn7)CC6)c5)n4)cccc3C2=O)C(=O)N1]

GS-029 was synthesized following the similar procedure for preparing GS-028 (50 mg, yield: 42.1%). MS (ESI) m/z=720.8 [M+H]⁺.

### Example 26. 3-(1-Oxo-4-((2-((4-((4-(4-(quinoxalin-2-yl)-1H-pyrazol-1-yl)piperidin-1-yl)methyl)phenyl)amino)pyrimidin-4-yl)amino)isoindolin-2-yl)piperidine-2,6-dione (GS-031)

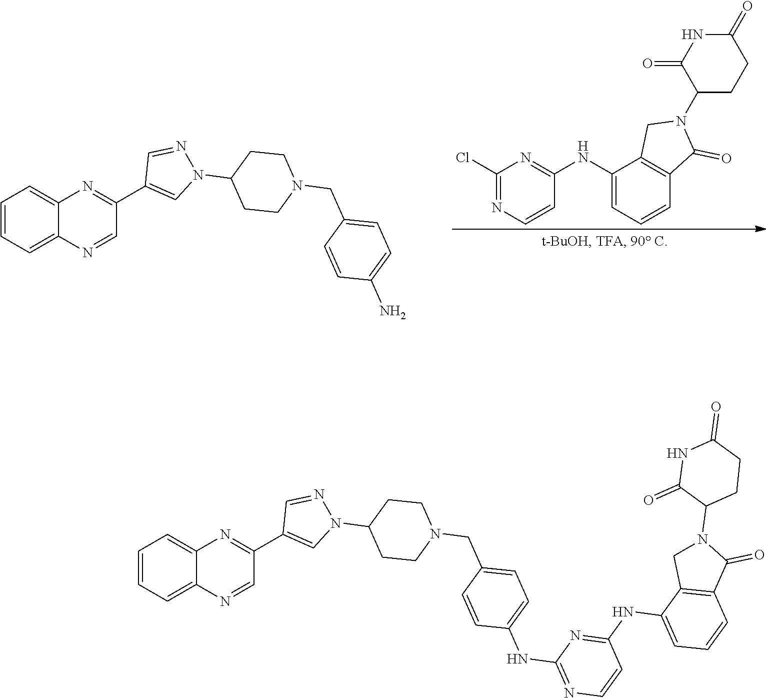 [SMILES: Nc1ccc(CN2CCC(n3cc(-c4cnc5ccccc5n4)cn3)CC2)cc1 ; O=C1CCC(N2Cc3c(Nc4ccnc(Cl)n4)cccc3C2=O)C(=O)N1 ; O=C1CCC(N2Cc3c(Nc4ccnc(Nc5ccc(CN6CCC(n7cc(-c8cnc9ccccc9n8)cn7)CC6)cc5)n4)cccc3C2=O)C(=O)N1]

GS-031 was synthesized following the similar procedure for preparing GS-028 (50 mg, yield: 42.1%). MS (ESI) m/z=720.7 [M+H]⁺.

### Example 27. 3-(1-Oxo-4-((2-((3-(4-(4-(quinoxalin-2-yl)-1H-pyrazol-1-yl)piperidin-1-yl)phenyl)amino)pyrimidin-4-yl)amino)isoindolin-2-yl)piperidine-2,6-dione (GS-025)

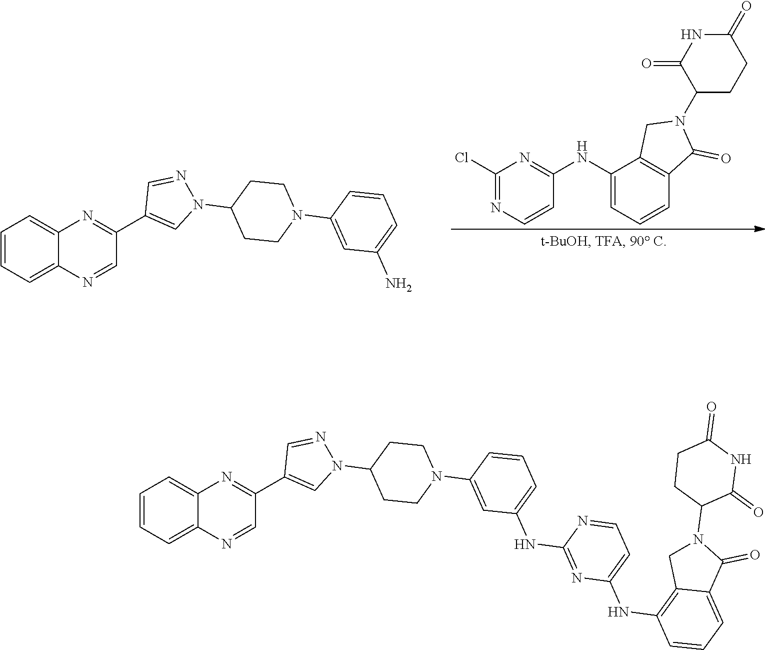 [SMILES: Nc1cccc(N2CCC(n3cc(-c4cnc5ccccc5n4)cn3)CC2)c1 ; O=C1CCC(N2Cc3c(Nc4ccnc(Cl)n4)cccc3C2=O)C(=O)N1 ; O=C1CCC(N2Cc3c(Nc4ccnc(Nc5cccc(N6CCC(n7cc(-c8cnc9ccccc9n8)cn7)CC6)c5)n4)cccc3C2=O)C(=O)N1]

GS-025 was synthesized following the similar procedure for preparing GS-028 (18 mg, yield: 79.7%). MS (ESI) m/z=706.7 [M+H]⁺.

### Example 28. 3-(4-((2-((4-(4-(4-(8-Bromoquinoxalin-2-yl)-1H-pyrazol-1-yl)piperidin-1-yl)phenyl)amino)pyrimidin-4-yl)amino)-1-oxoisoindolin-2-yl)piperidine-2,6-dione (GS-026)

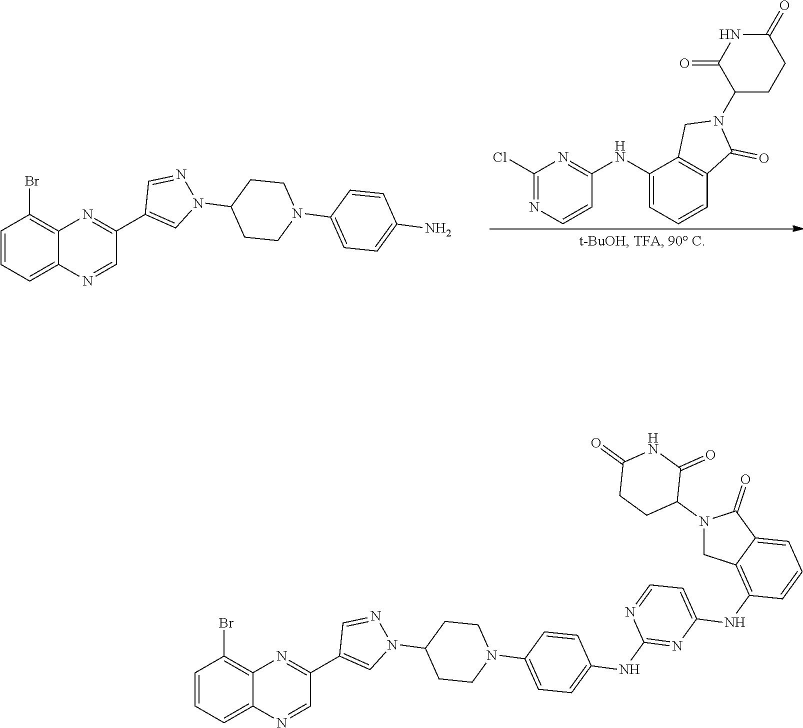 [SMILES: Nc1ccc(N2CCC(n3cc(-c4cnc5cccc(Br)c5n4)cn3)CC2)cc1 ; O=C1CCC(N2Cc3c(Nc4ccnc(Cl)n4)cccc3C2=O)C(=O)N1 ; O=C1CCC(N2Cc3c(Nc4ccnc(Nc5ccc(N6CCC(n7cc(-c8cnc9cccc(Br)c9n8)cn7)CC6)cc5)n4)cccc3C2=O)C(=O)N1]

GS-026 was synthesized following the similar procedure for preparing GS-028 (25 mg, yield: 59.7%). MS (ESI) m/z=784.5 [M+H]⁺.

### Example 29. 3-(1-Oxo-4-((2-((4-(4-(4-(quinoxalin-2-yl)-1H-pyrazol-1-yl)piperidin-1-yl)phenyl)amino)pyrimidin-4-yl)amino)isoindolin-2-yl)piperidine-2,6-dione (GS-027)

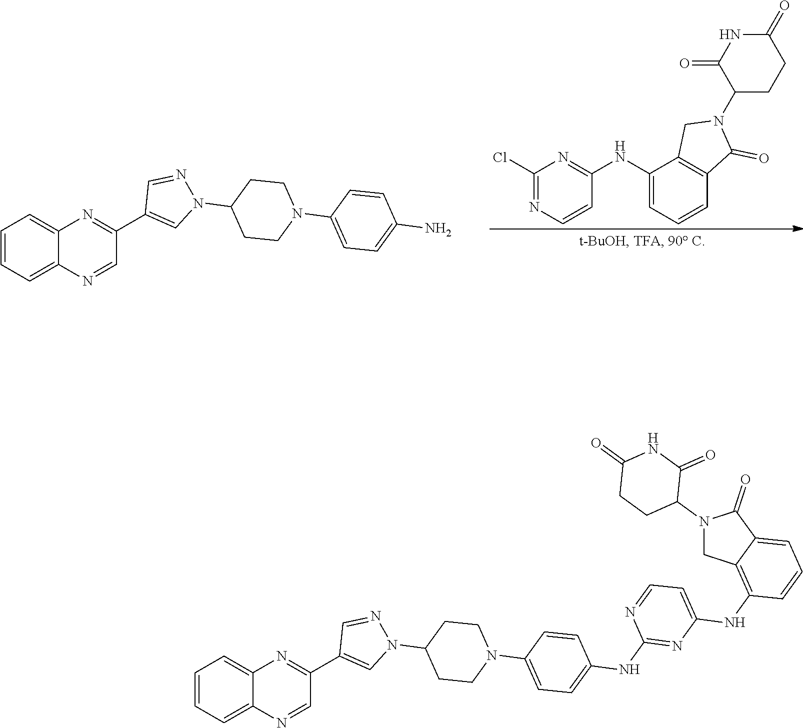 [SMILES: Nc1ccc(N2CCC(n3cc(-c4cnc5ccccc5n4)cn3)CC2)cc1 ; O=C1CCC(N2Cc3c(Nc4ccnc(Cl)n4)cccc3C2=O)C(=O)N1 ; O=C1CCC(N2Cc3c(Nc4ccnc(Nc5ccc(N6CCC(n7cc(-c8cnc9ccccc9n8)cn7)CC6)cc5)n4)cccc3C2=O)C(=O)N1]

GS-027 was synthesized following the similar procedure for preparing GS-028 (37 mg, yield: 56%). MS (ESI) m/z=706.7 [M+H]⁺.

### Example 30. 3-(5-((2-((2-(4-(4-(8-Bromoquinoxalin-2-yl)-1H-pyrazol-1-yl)piperidin-1-yl)-2,3-dihydro-1H-inden-5-yl)amino)pyrimidin-4-yl)amino)-1-oxoisoindolin-2-yl)piperidine-2,6-dione (GS-072)

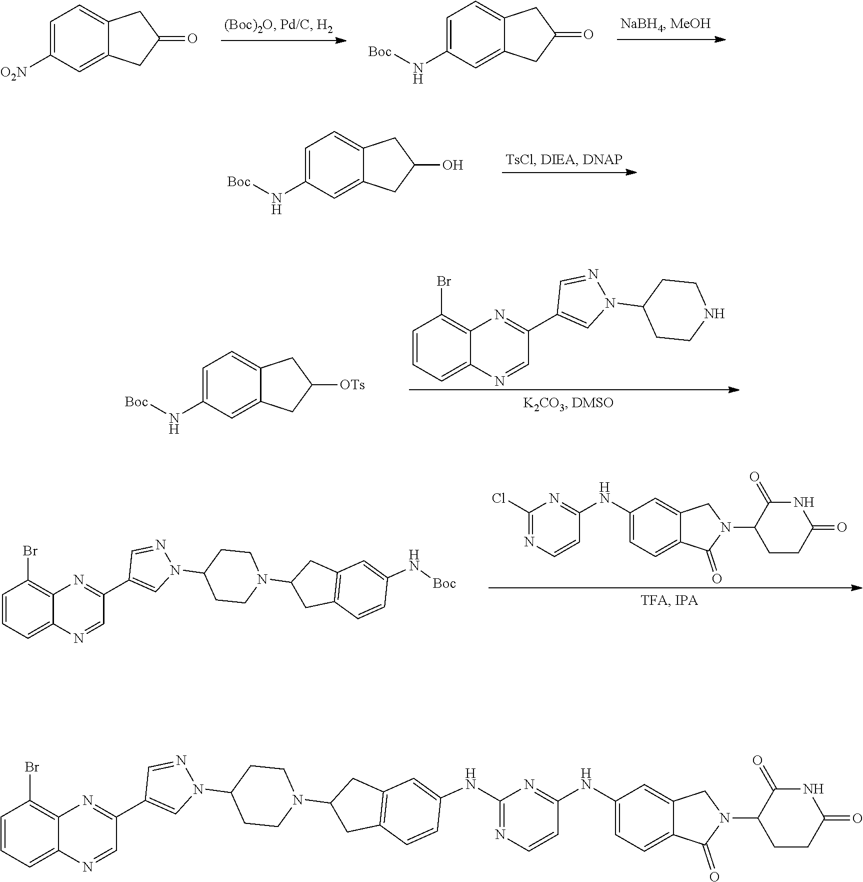

Step 1: Synthesis of tert-butyl (2-oxo-2,3-dihydro-1H-inden-5-yl)carbamate

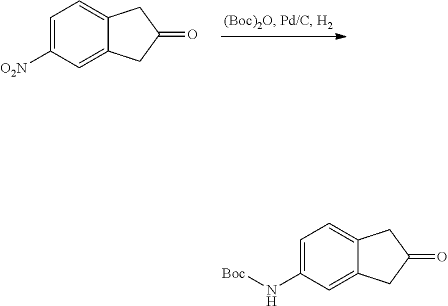 [SMILES: O=C1Cc2ccc([N+](=O)[O-])cc2C1 ; CC(C)(C)OC(=O)Nc1ccc2c(c1)CC(=O)C2]

To a solution of 5-nitro-1,3-dihydro-2H-inden-2-one (500.0 mg, 2.82 mmol) in THF (15 mL) were added Pd/C (10%, 100 mg) and (Boc)₂O (609.1 mg, 2.82 mmol) under nitrogen atmosphere. The mixture was purged with hydrogen and stirred at rt for 2 h under hydrogen atmosphere using a hydrogen balloon. The reaction mixture was filtered through a Celite pad, and the filtrate was concentrated. The resulting residue was purified by silica gel column chromatography (petroleum ether/EtOAc=3:1) to provide the title compound (344.2 mg, yield: 49.3%) as a white solid. MS (ESI) m/z=248.1 [M+H]⁺.

Step 2: Synthesis of tert-butyl (2-hydroxy-2,3-dihydro-1H-inden-5-yl)carbamate

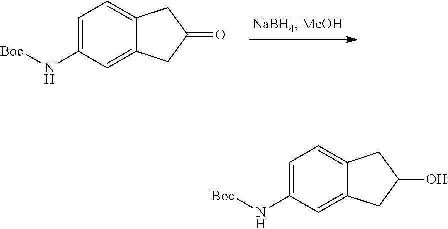 [SMILES: CC(C)(C)OC(=O)Nc1ccc2c(c1)CC(=O)C2 ; CC(C)(C)OC(=O)Nc1ccc2c(c1)CC(O)C2]

To a solution of tert-butyl (2-oxo-2,3-dihydro-1H-inden-5-yl)carbamate (344.2 mg, 1.39 mmol) in MeOH (10 mL) was added NaBH₄ (79.2 mg, 2.08 mmol) at 0° C. The mixture was stirred at rt for 1 h. The reaction mixture was quenched with water and extracted with DCM (20 mL×3). The combined organic layers were dried over Na₂SO₄, filtered, and concentrated. The resulting residue was used in the next step directly without further purification. MS (ESI) m/z=250.1 [M+H]⁺.

Step 3: Synthesis of 5-((tert-butoxycarbonyl)amino)-2,3-dihydro-1H-inden-2-yl 4-methylbenzenesulfonate

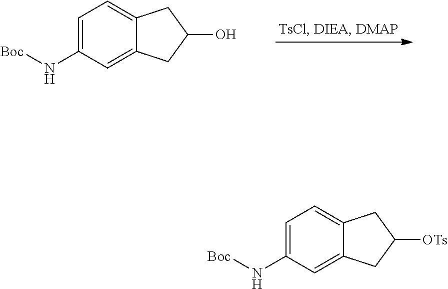 [SMILES: CC(C)(C)OC(=O)Nc1ccc2c(c1)CC(O)C2 ; Cc1ccc(S(=O)(=O)OC2Cc3ccc(NC(=O)OC(C)(C)C)cc3C2)cc1]

To a solution of tert-butyl (2-hydroxy-2,3-dihydro-1H-inden-5-yl)carbamate (294.5 mg, 1.17 mmol) and TsCl (444.6 mg, 2.34 mmol) in DCM (10 mL) were added DMAP (13.4 mg, 0.11 mmol) and TEA (590.8 mg, 5.85 mmol). After the reaction was stirred at rt for 3 h, it was diluted with DCM (20 mL), and washed with H₂O and brine. The organic layer was dried over Na₂SO₄, filtered, and concentrated. The resulting residue was purified by silica gel column chromatography (petroleum ether/EtOAc=3:1) to provide the title compound (307.7 mg, yield: 70.2%) as a white solid. MS (ESI) m/z=404.5 [M+H]⁺.

Step 4: Synthesis of tert-butyl (2-(4-(4-(8-bromoquinoxalin-2-yl)-1H-pyrazol-1-yl)piperidin-1-yl)-2,3-dihydro-1H-inden-5-yl)carbamate

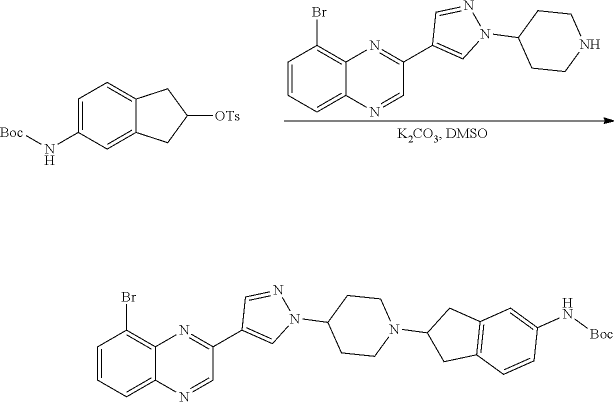 [SMILES: Cc1ccc(S(=O)(=O)OC2Cc3ccc(NC(=O)OC(C)(C)C)cc3C2)cc1 ; Brc1cccc2ncc(-c3cnn(C4CCNCC4)c3)nc12 ; CC(C)(C)OC(=O)Nc1ccc2c(c1)CC(N1CCC(n3cc(-c4cnc5cccc(Br)c5n4)cn3)CC1)C2]

To a solution of 5-((tert-butoxycarbonyl)amino)-2,3-dihydro-1H-inden-2-yl 4-methylbenzenesulfonate (30.0 mg, 0.07 mmol) in DMSO (1 mL) were added 8-bromo-2-(1-(piperidin-4-yl)-1H-pyrazol-4-yl)quinoxaline (24.9 mg, 0.07 mmol) and K₂CO₃ (29.1 mg, 0.21 mmol). After the reaction was stirred at 60° C. for 2 h, it was purified by C18 column chromatography to provide the title compound (12.8 mg, yield: 31.2%) as a light-yellow solid. MS (ESI) m/z=589.3 [M+H]⁺.

Step 5: Synthesis of 3-(5-((2-((2-(4-(4-(8-bromoquinoxalin-2-yl)-1H-pyrazol-1-yl)piperidin-1-yl)-2,3-dihydro-1H-inden-5-yl)amino)pyrimidin-4-yl)amino)-1-oxoisoindolin-2-yl)piperidine-2,6-dione

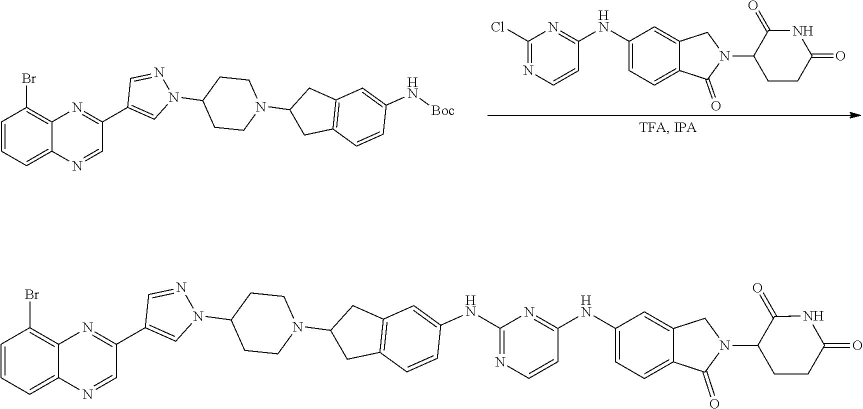 [SMILES: CC(C)(C)OC(=O)Nc1ccc2c(c1)CC(N1CCC(n3cc(-c4cnc5cccc(Br)c5n4)cn3)CC1)C2 ; O=C1CCC(N2Cc3cc(Nc4ccnc(Cl)n4)ccc3C2=O)C(=O)N1 ; O=C1CCC(N2Cc3cc(Nc4ccnc(Nc5ccc6c(c5)CC(N5CCC(n7cc(-c8cnc9cccc(Br)c9n8)cn7)CC5)C6)n4)ccc3C2=O)C(=O)N1]

To a solution of tert-butyl (2-(4-(4-(8-bromoquinoxalin-2-yl)-1H-pyrazol-1-yl)piperidin-1-yl)-2,3-dihydro-1H-inden-5-yl)carbamate (12.8 mg, 0.02 mmol) in IPA (1 mL) were added 3-(5-((2-chloropyrimidin-4-yl)amino)-1-oxoisoindolin-2-yl)piperidine-2,6-dione (7.4 mg, 0.02 mmol) and TFA (26.2 mg, 0.23 mmol). The reaction mixture was irradiated at 80° C. under microwave irradiation reactor for 3 h. The resulting mixture was purified by reverse phase flash chromatography to provide the title compound (4.4 mg, yield: 26.8%) as a yellow solid. MS (ESI) m/z=824.5 [M+H]⁺.

### Example 31. 3-(1-Oxo-5-((2-((2-(4-(4-(quinoxalin-2-yl)-1H-pyrazol-1-yl)piperidin-1-yl)-2,3-dihydro-1H-inden-5-yl)amino)pyrimidin-4-yl)amino)isoindolin-2-yl)piperidine-2,6-dione (GS-073)

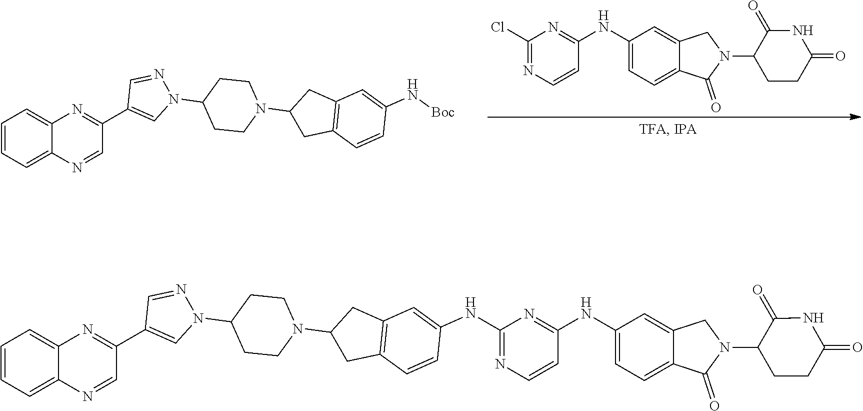 [SMILES: CC(C)(C)OC(=O)Nc1ccc2c(c1)CC(N1CCC(n3cc(-c4cnc5ccccc5n4)cn3)CC1)C2 ; O=C1CCC(N2Cc3cc(Nc4ccnc(Cl)n4)ccc3C2=O)C(=O)N1 ; O=C1CCC(N2Cc3cc(Nc4ccnc(Nc5ccc6c(c5)CC(N5CCC(n7cc(-c8cnc9ccccc9n8)cn7)CC5)C6)n4)ccc3C2=O)C(=O)N1]

GS-073 was synthesized following the similar procedure for preparing GS-072 (3.7 mg, yield: 24.8%). MS (ESI) m/z=746.7 [M+H]⁺.

### Example 32. 3-(1-Oxo-4-((2-(3-(4-(4-(quinoxalin-2-yl)-1H-pyrazol-1-yl)piperidin-1-yl)pyrrolidin-1-yl)pyrimidin-4-yl)amino)isoindolin-2-yl)piperidine-2,6-dione (GS-074)

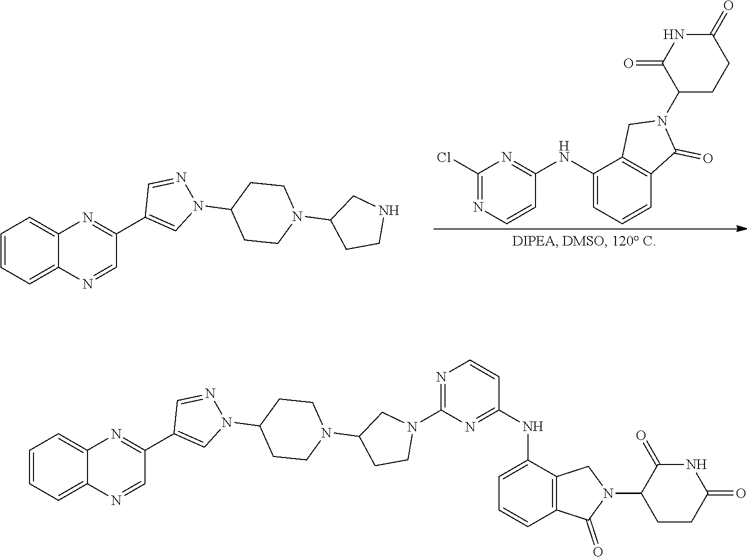 [SMILES: c1ccc2nc(-c3cnn(C4CCN(C5CCNC5)CC4)c3)cnc2c1 ; O=C1CCC(N2Cc3c(Nc4ccnc(Cl)n4)cccc3C2=O)C(=O)N1 ; O=C1CCC(N2Cc3c(Nc4ccnc(N5CCC(N6CCC(n7cc(-c8cnc9ccccc9n8)cn7)CC6)C5)n4)cccc3C2=O)C(=O)N1]

To a solution of 2-[1-(1-pyrrolidin-3-yl-4-piperidyl)pyrazol-4-yl]quinoxaline (30 mg, 86.10 μmol) and 3-[4-[(2-chloropyrimidin-4-yl)amino]-1-oxo-isoindolin-2-yl]piperidine-2,6-dione (32.01 mg, 86.10 μmol) in DMSO (5 mL) was added DIPEA (66.76 mg, 516.58 μmol) at rt. After the mixture was stirred at 120° C. for 16 h, it was cool to rt and purified by prep-HPLC to provide 100 mg crude product which was further purified by prep-TLC (DCM/MeOH=8/1) to provide the title compound (45 mg, 76.4% yield) as a white solid. MS (ESI) m/z=684.7 [M+H]⁺.

### Example 33. 3-(1-Oxo-4-((2-((3-(4-phenylpiperidin-1-yl)phenyl)amino)pyrimidin-4-yl amino)isoindolin-2-yl)piperidine-2,6-dione (GS-075)

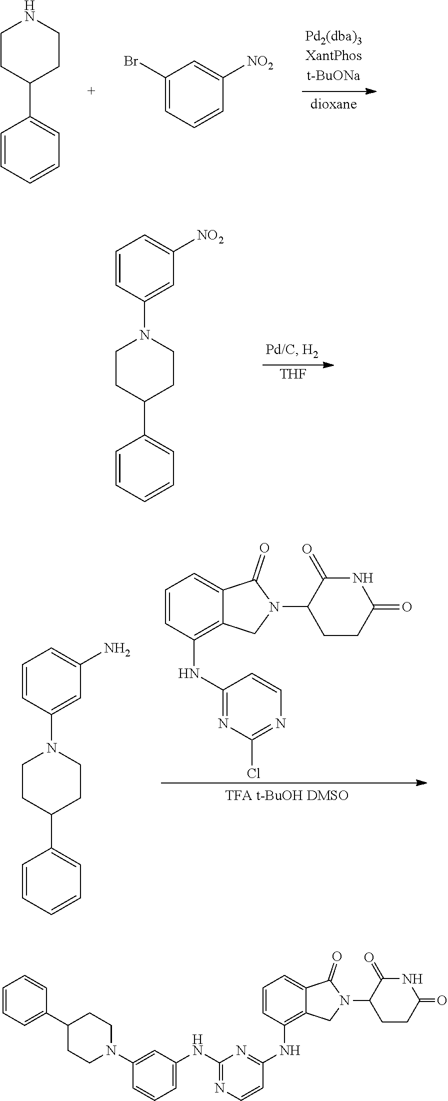

Step 1: Synthesis of 1-(3-nitrophenyl)-4-phenylpiperidine

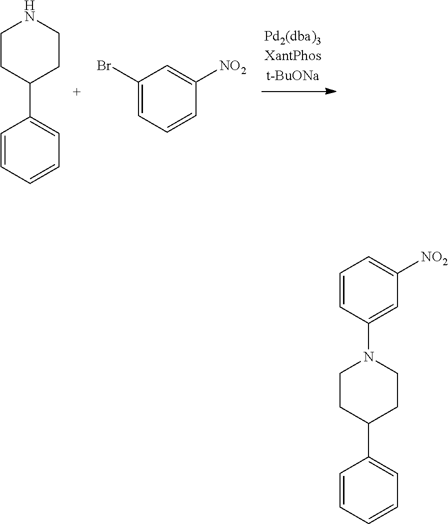 [SMILES: c1ccc(C2CCNCC2)cc1 ; O=[N+]([O-])c1cccc(Br)c1 ; O=[N+]([O-])c1cccc(N2CCC(c3ccccc3)CC2)c1]

To a solution of 4-phenylpiperidine (100 mg, 0.62 mmol) in Dioxane (10 mL) were added 1-bromo-3-nitro-benzene (188 mg, 0.93 mol), Pd₂(dba)₃ (114 mg, 0.124 mmol), Xantphos (108 mg, 0.19 mmol) and t-BuONa (179 mg, 1.86 mmol). The mixture was irradiated at 100° C. under N₂ for 2 h under microwave irradiation reactor. The mixture was purified by silica gel flash chromatography to provide the title compound (130 mg, 74.2% yield) as a yellow solid.

Step 2: Synthesis of 3-(4-phenylpiperidin-1-yl)aniline

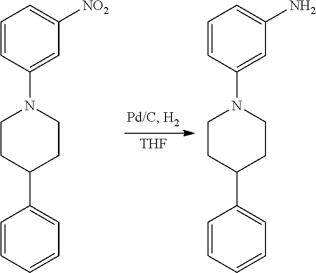 [SMILES: O=[N+]([O-])c1cccc(N2CCC(c3ccccc3)CC2)c1 ; Nc1cccc(N2CCC(c3ccccc3)CC2)c1 ; "THF"]

To a solution of 1-(3-nitrophenyl)-4-phenyl-piperidine (130 mg, 0.46 mmol) in THE (5 mL) was added Pd/C (13 mg). The reaction was stirred at 25° C. for 2 h under H₂. The reaction mixture was filtered over Celite. The filtrate was concentrated to provide the title compound (106 mg, 91.2% yield) as a white solid which was used directly in the next step without further purification.

Step 3: Synthesis of 3-(1-oxo-4-((2-((3-(4-phenylpiperidin-1-yl)phenyl)amino)pyrimidin-4-yl)amino)isoindolin-2-yl)piperidine-2,6-dione

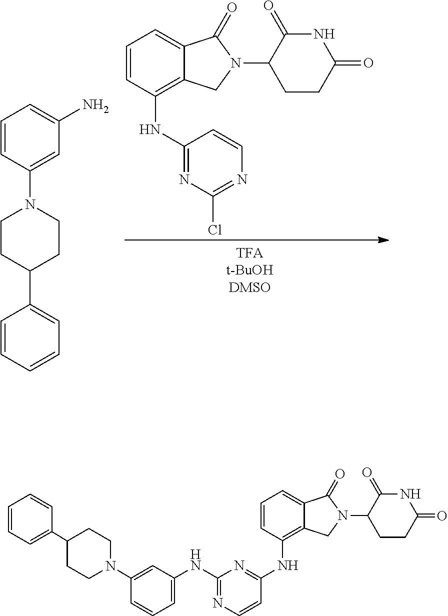 [SMILES: Nc1cccc(N2CCC(c3ccccc3)CC2)c1 ; O=C1CCC(N2Cc3c(Nc4ccnc(Cl)n4)cccc3C2=O)C(=O)N1 ; O=C1CCC(N2Cc3c(Nc4ccnc(Nc5cccc(N6CCC(c7ccccc7)CC6)c5)n4)cccc3C2=O)C(=O)N1]

A mixture of 3-(4-phenyl-1-piperidyl)aniline (20 mg, 0.079 mmol), 3-[4-[(2-chloropyrimidin-4-yl)amino]-1-oxo-isoindolin-2-yl]piperidine-2,6-dione (44 mg, 0.119 mmol) and TFA (27 mg, 0.237 mmol) in t-BuOH (1.0 mL) and DMSO (0.2 mL) was stirred at 90° C. for 3 h. The reaction was concentrated. The resulting residue was purified by reverse phase column chromatography to provide the title compound (19 mg, 40.8% yield) as a white solid. MS (ESI) m/z=588.5 [M+H]⁺.

### Example 34. 3-(1-Oxo-4-((2-((3-(4-(pyridin-2-yl)piperazin-1-yl)phenyl)amino)pyrimidin-4-yl)amino)isoindolin-2-yl)piperidine-2,6-dione (GS-076)

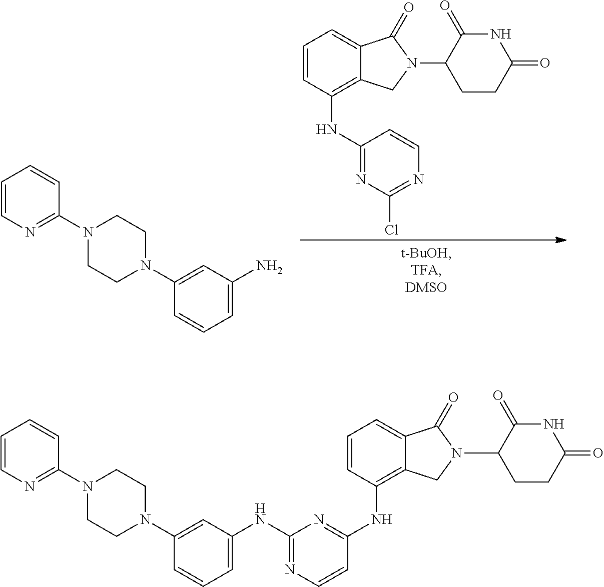 [SMILES: Nc1cccc(N2CCN(c3ccccn3)CC2)c1 ; O=C1CCC(N2Cc3c(Nc4ccnc(Cl)n4)cccc3C2=O)C(=O)N1 ; O=C1CCC(N2Cc3c(Nc4ccnc(Nc5cccc(N6CCN(c7ccccn7)CC6)c5)n4)cccc3C2=O)C(=O)N1]

GS-076 was synthesized following the similar procedure for preparing GS-075 (29 mg, yield: 62.5%). MS (ESI) m/z=590.5 [M+H]⁺.

### Example 35. 3-(1-Oxo-4-((2-((3-(4-(pyridin-2-yl)piperidin-1-yl)phenyl)amino)pyrimidin-4-yl)amino)isoindolin-2-yl)piperidine-2,6-dione (GS-077)

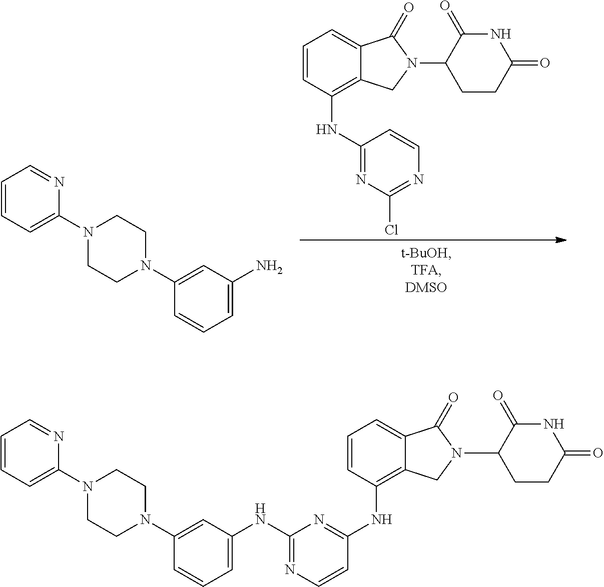 [SMILES: Nc1cccc(N2CCN(c3ccccn3)CC2)c1 ; O=C1CCC(N2Cc3c(Nc4ccnc(Cl)n4)cccc3C2=O)C(=O)N1 ; O=C1CCC(N2Cc3c(Nc4ccnc(Nc5cccc(N6CCN(c7ccccn7)CC6)c5)n4)cccc3C2=O)C(=O)N1]

GS-077 was synthesized following the similar procedure for preparing GS-075 (4 mg, yield: 8.6%). MS (ESI) m/z=589.4 [M+H]⁺.

### Example 36. 3-(1-Oxo-4-((2-((3-(4-(pyrimidin-2-yl)piperazin-1-yl)phenyl)amino)pyrimidin-4-yl)amino)isoindolin-2-yl)piperidine-2,6-dione (GS-078)

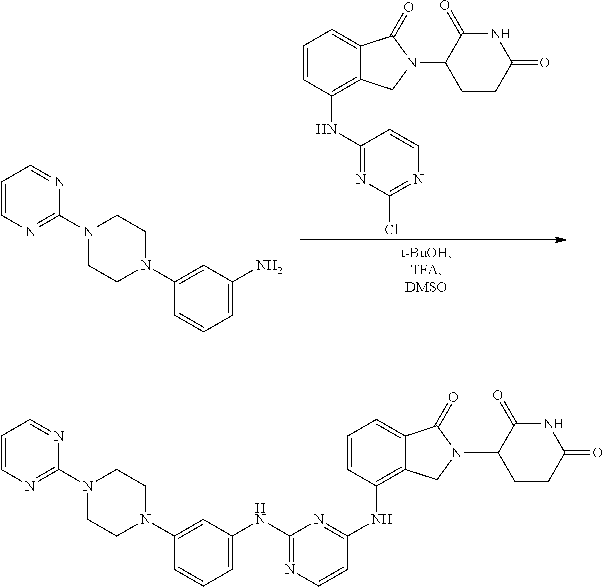 [SMILES: Nc1cccc(N2CCN(c3ncccn3)CC2)c1 ; O=C1CCC(N2Cc3c(Nc4ccnc(Cl)n4)cccc3C2=O)C(=O)N1 ; O=C1CCC(N2Cc3c(Nc4ccnc(Nc5cccc(N6CCN(c7ncccn7)CC6)c5)n4)cccc3C2=O)C(=O)N1]

GS-078 was synthesized following the similar procedure for preparing GS-075 (11 mg, yield: 23.8%). MS (ESI) m/z=591.5 [M+H]⁺.

### Example 37. 3-(1-Oxo-4-((2-((3-(4-(4-(quinoxalin-2-yl)-1H-pyrazol-1-yl)piperidine-1-carbonyl)phenyl)amino)pyrimidin-4-yl)amino)isoindolin-2-yl)piperidine-2,6-dione (GS-079)

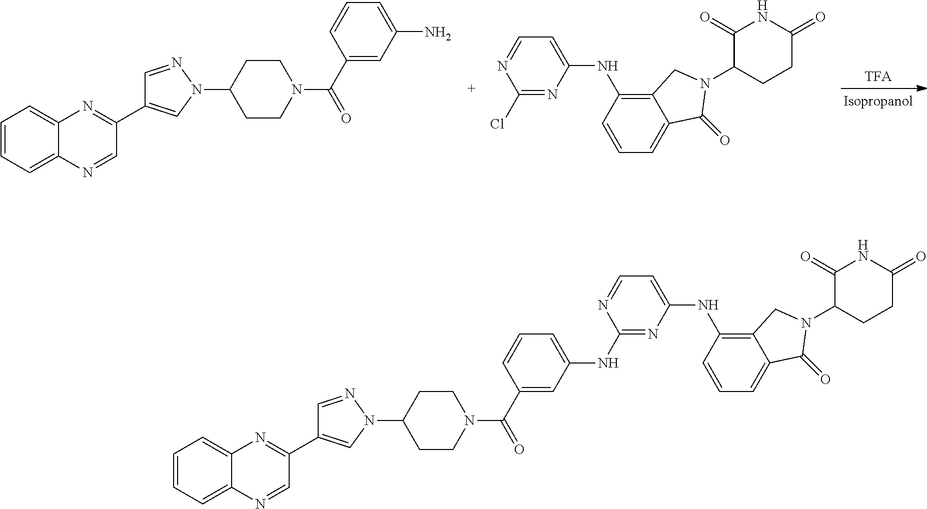 [SMILES: Nc1cccc(C(=O)N2CCC(n3cc(-c4cnc5ccccc5n4)cn3)CC2)c1 ; O=C1CCC(N2Cc3c(Nc4ccnc(Cl)n4)cccc3C2=O)C(=O)N1 ; O=C1CCC(N2Cc3c(Nc4ccnc(Nc5cccc(C(=O)N6CCC(n7cc(-c8cnc9ccccc9n8)cn7)CC6)c5)n4)cccc3C2=O)C(=O)N1]

GS-079 was synthesized following the similar procedure for preparing GS-075 (2.9 mg, yield: 19.6%). MS (ESI) m/z=734.7 [M+H]⁺.

### Example 38. 3-(1-Oxo-4-((2-((3-(4-(pyridin-2-yl)piperazine-1-carbonyl)phenyl)amino)pyrimidin-4-yl)amino)isoindolin-2-yl)piperidine-2,6-dione (GS-080)

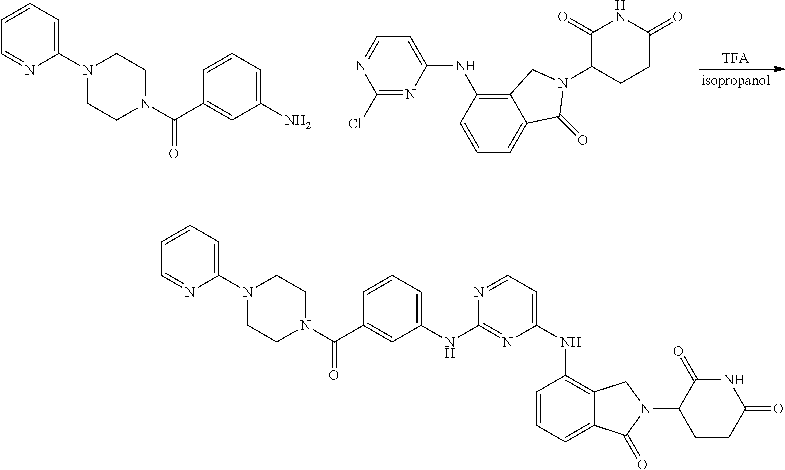 [SMILES: Nc1cccc(C(=O)N2CCN(c3ccccn3)CC2)c1 ; O=C1CCC(N2Cc3c(Nc4ccnc(Cl)n4)cccc3C2=O)C(=O)N1 ; O=C1CCC(N2Cc3c(Nc4ccnc(Nc5cccc(C(=O)N6CCN(c7ccccn7)CC6)c5)n4)cccc3C2=O)C(=O)N1]

GS-080 was synthesized following the similar procedure for preparing GS-075 (3.8 mg, yield: 28.9%). MS (ESI) m/z=618.5 [M+H]⁺.

### Example 39. 3-(1-Oxo-4-((2-(4-(4-(quinoxalin-2-yl)-1H-pyrazol-1-yl)piperidin-1-yl)pyrimidin-4-yl)amino)isoindolin-2-yl)piperidine-2,6-dione (GS-081)

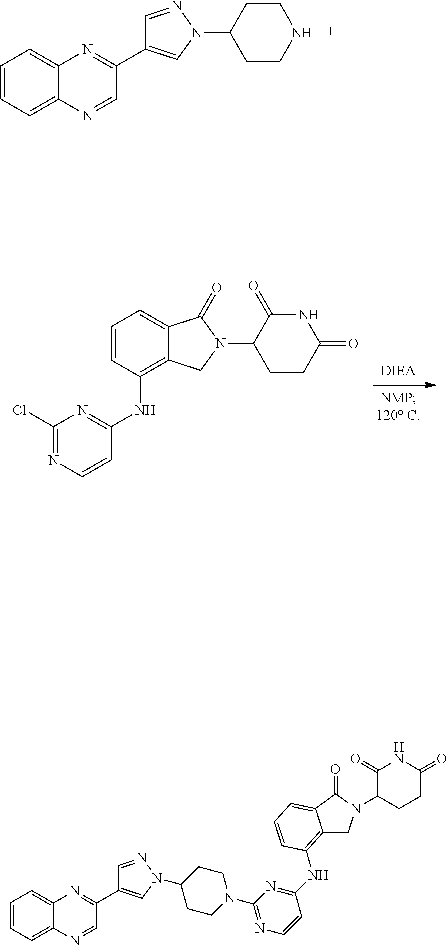 [SMILES: c1ccc2nc(-c3cnn(C4CCNCC4)c3)cnc2c1 ; O=C1CCC(N2Cc3c(Nc4ccnc(Cl)n4)cccc3C2=O)C(=O)N1 ; O=C1CCC(N2Cc3c(Nc4ccnc(N5CCC(n6cc(-c7cnc8ccccc8n7)cn6)CC5)n4)cccc3C2=O)C(=O)N1]

GS-081 was synthesized following the similar procedure for preparing GS-074 (10 mg, yield: 54.3%). MS (ESI) m/z=615.5 [M+H]⁺.

### Example 40. N-((2-(2,6-Dioxopiperidin-3-yl)-1-oxoisoindolin-5-yl)methyl)-2-(3-(4-(4-(quinoxalin-2-yl)-1H-pyrazol-1-yl)piperidin-1-yl)cyclobutyl)acetamide (GS-082)

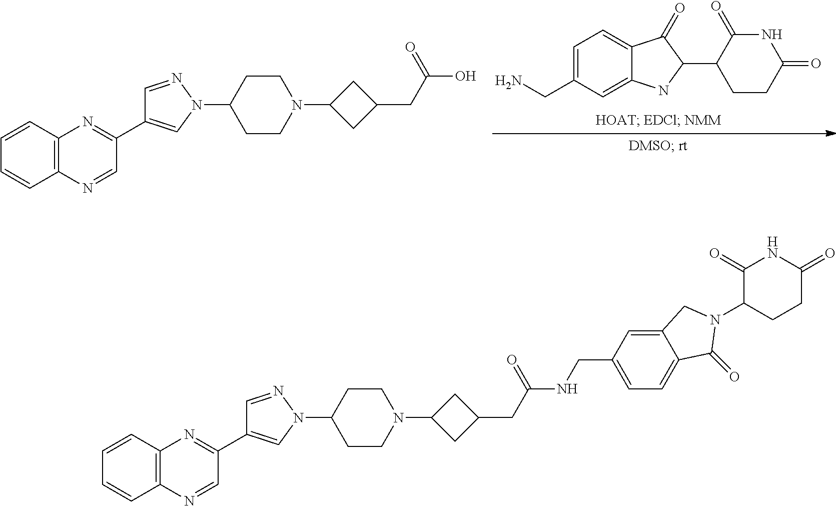 [SMILES: O=C(O)CC1CC(N2CCC(n3cc(-c4cnc5ccccc5n4)cn3)CC2)C1 ; NCc1ccc2c(c1)NC(C1CCC(=O)NC1=O)C2=O ; O=C(CC1CC(N2CCC(n3cc(-c4cnc5ccccc5n4)cn3)CC2)C1)NCc1ccc2c(c1)CN(C1CCC(=O)NC1=O)C2=O]

GS-082 was synthesized following the similar procedure for preparing GS-004 (22 mg, yield: 48.4%). MS (ESI) m/z=647.3 [M+H]⁺.

### Example 41. N-((2-(2,6-Dioxopiperidin-3-yl)-1-oxoisoindolin-5-yl)methyl)-2-(2-(4-(4-(quinoxalin-2-yl)-1H-pyrazol-1-yl)piperidin-1-yl)cyclopropyl)acetamide (GS-083)

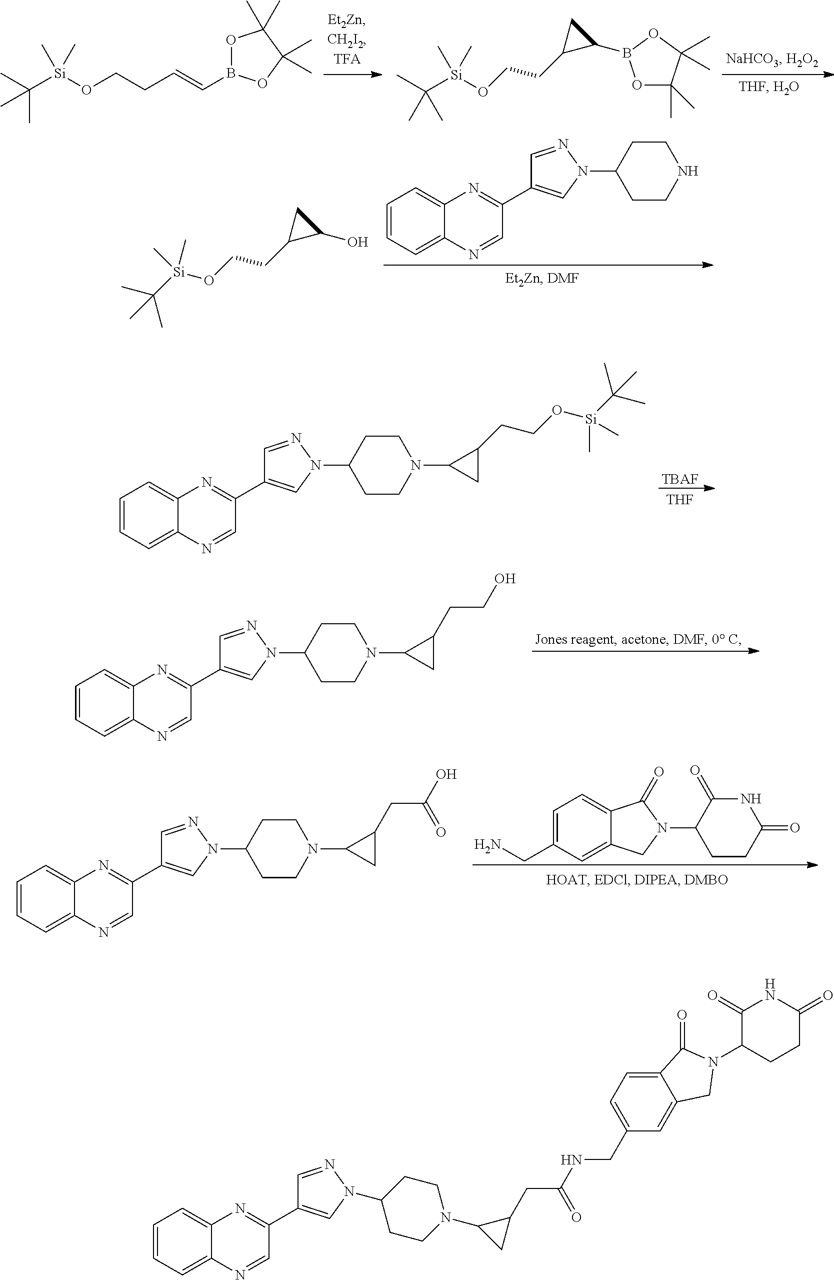

Step 1: Synthesis of tert-butyldimethyl(2-(2-(4,4,5,5-tetramethyl-1,3,2-dioxaborolan-2-yl)cyclopropyl)ethoxy)silane

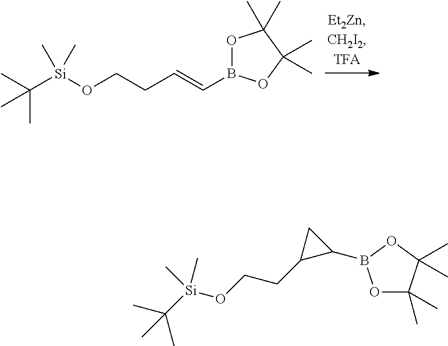

To a solution of tert-butyldimethyl((4-(4,4,5,5-tetramethyl-1,3,2-dioxaborolan-2-yl)but-3-en-1-yl)oxy)silane (2.0 g, 6.40 mmol) in DCM (30 mL) was added Et₂Zn (32.02 mmol, 32 mL) at 0° C. After the mixture was stirred at 0° C. for 0.5 h, CH₂I₂ (8.58 g, 32.02 mmol) and TFA (3.65 g, 32.02 mmol) were added at 0° C. The resulting mixture was stirred at 0° C. for 1 h, before it was stirred at rt for 2 h. The reaction was quenched with aq. NH₄Cl, extracted with DCM, and concentrated. The resulting residue was purified by silica gel chromatography (petroleum ether/EtOAc=10/1) to provide the title compound (2.0 g, 95.7% yield) as a colorless oil.

Step 2: Synthesis of 2-(2-((tert-butyldimethylsilyl)oxy)ethyl)cyclopropan-1-ol

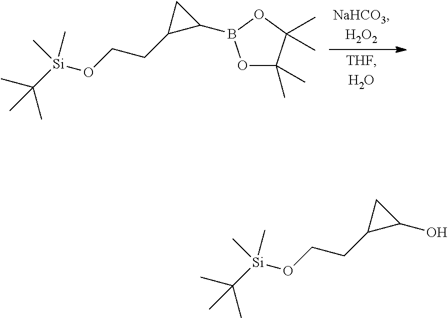

To a solution of tert-butyldimethyl(2-(2-(4,4,5,5-tetramethyl-1,3,2-dioxaborolan-2-yl)cyclopropyl)ethoxy)silane (2.0 g, 6.13 mmol) in THF (50 mL) were added NaHCO₃ (6.13 mmol, 30 mL) and H₂O₂ (6.13 mmol, 20 mL) at 0° C. After the reaction mixture was stirred at 0° C. for 1 h, it was extracted with DCM, and concentrated. The resulting residue was purified by silica gel chromatography (petroleum ether/EtOAc=3/1) to provide the title compound (1.2 g, 90.5% yield) as a colorless oil.

Step 3: Synthesis of 2-(1-(1-(2-(2-((tert-butyldimethylsilyl)oxy)ethyl)cyclopropyl)piperidin-4-yl)-1H-pyrazol-4-yl)quinoxaline

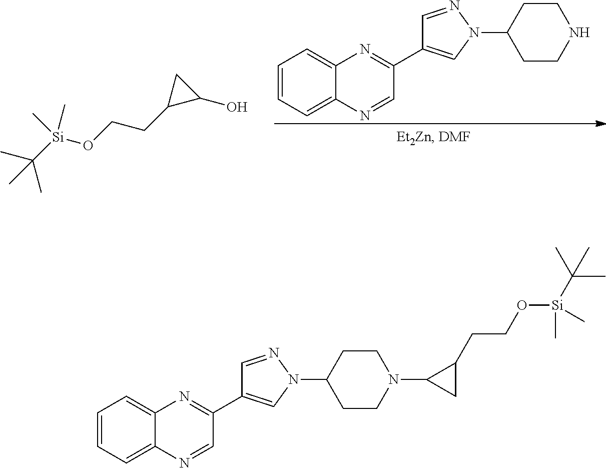

To a solution of 2-(1-(piperidin-4-yl)-1H-pyrazol-4-yl)quinoxaline (280 mg, 1.00 mmol) and 2-(2-((tert-butyldimethylsilyl)oxy)ethyl)cyclopropan-1-ol (401.39 mg, 1.85 mmol) in DMF (20 mL) was added Zn(Et)₂ (1.00 mmol, 4 mL) at rt. After the mixture was stirred at 110° C. for 48 h, it was quenched with aq. NH₄Cl, extracted with DCM, and concentrated. The resulting residue was purified by silica gel chromatography (DCM/MeOH=15/1) to provide the title compound (150 mg, 31.3% yield). MS (ESI) m/z=478.6 [M+H]⁺.

Step 4: Synthesis of 2-(2-(4-(4-(quinoxalin-2-yl)-1H-pyrazol-1-yl)piperidin-1-yl)cyclopropyl)ethan-1-ol

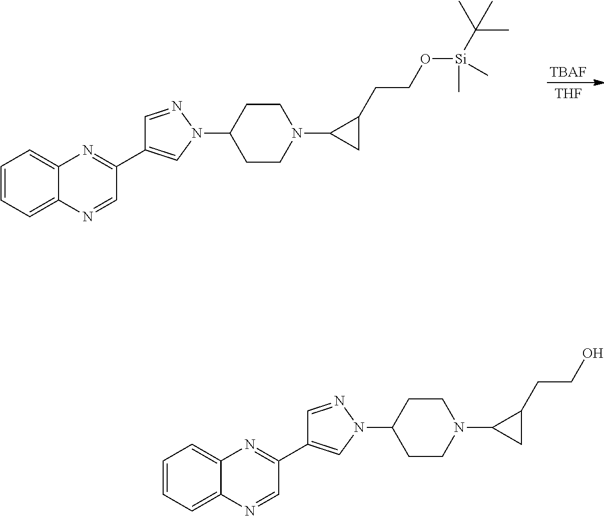

To a solution of 2-(1-(1-(2-(2-((tert-butyldimethylsilyl)oxy)ethyl)cyclopropyl)piperidin-4-yl)-1H-pyrazol-4-yl)quinoxaline (150 mg, 313.99 μmol) in THF (5 mL) was added TBAF (82.10 mg, 313.99 μmol, 2 mL) at rt. After the mixture was stirred at rt for 0.5 h, it was concentrated. The resulting residue was purified by prep-HPLC to provide the title compound (100 mg, 66.7% yield) as a yellow solid. MS (ESI) m/z=364.4 [M+H]⁺.

Step 5: Synthesis of 2-(2-(4-(4-(quinoxalin-2-yl)-1H-pyrazol-1-yl)piperidin-1-yl)cyclopropyl)acetic acid

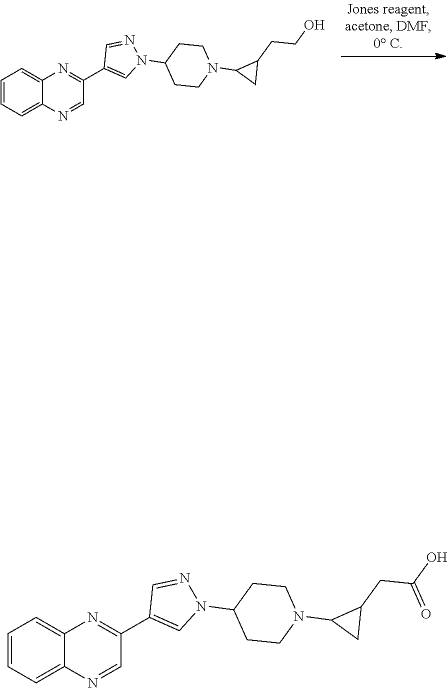

To a solution of 2-(2-(4-(4-(quinoxalin-2-yl)-1H-pyrazol-1-yl)piperidin-1-yl)cyclopropyl)ethan-1-ol (100 mg, 275.14 μmol) in acetone (3 mL) and DMF (1 mL) was added Jones reagent (0.4 mL) at 0° C. The reaction mixture was stirred at 0° C. for 3 h. The reaction mixture was purified by prep-HPLC to provide the title compound (35 mg, 33.7% yield). MS (ESI) m/z=378.3 [M+H]⁺.

Step 6: Synthesis of N-((2-(2,6-dioxopiperidin-3-yl)-1-oxoisoindolin-5-yl)methyl)-2-(2-(4-(4-(quinoxalin-2-yl)-1H-pyrazol-1-yl)piperidin-1-yl)cyclopropyl)acetamide

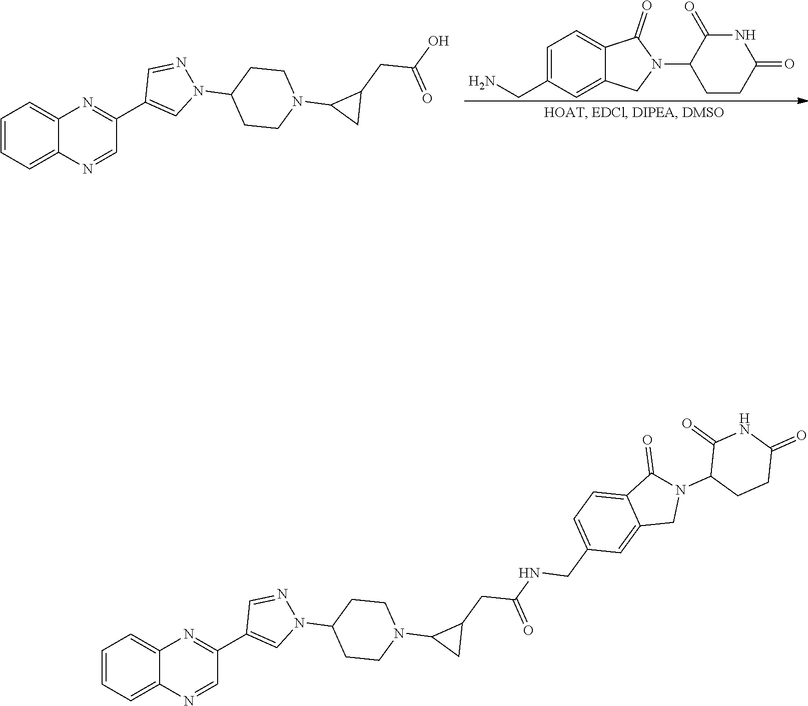 [SMILES: O=C(O)CC1CC1N1CCC(n2cc(-c3cnc4ccccc4n3)cn2)CC1 ; NCc1ccc2c(c1)CN(C1CCC(=O)NC1=O)C2=O ; O=C(CC1CC1N1CCC(n2cc(-c3cnc4ccccc4n3)cn2)CC1)NCc1ccc2c(c1)CN(C1CCC(=O)NC1=O)C2=O]

GS-083 was synthesized following the similar procedure for preparing GS-001 (30 mg, yield: 38.8%). MS (ESI) m/z=633.7 [M+H]⁺.

### Example 42. 3-(1-Oxo-4-((2-((3-(4-(quinoxalin-2-yl)-1H-pyrazol-1-yl)phenyl)amino)pyrimidin-4-yl)amino)isoindolin-2-yl)piperidine-2,6-dione (GS-084)

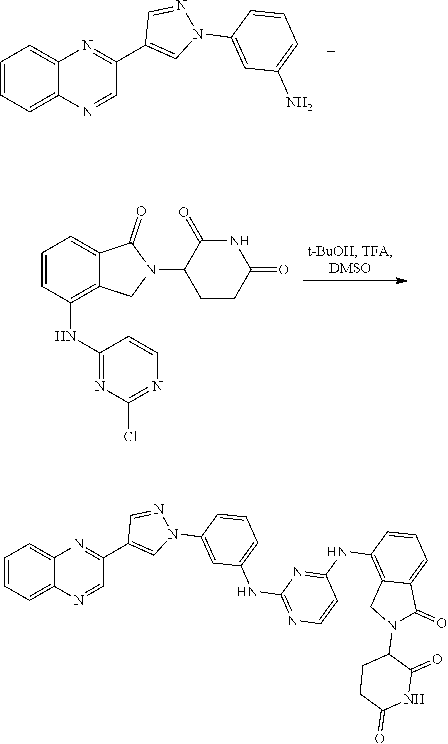

GS-084 was synthesized following the similar procedure for preparing GS-075 (9.5 mg, yield: 22.1%). MS (ESI) m/z=623.4 [M+H]⁺.

### Example 43. 3-(1-Oxo-4-((2-((3-oxo-3-(4-(4-(quinoxalin-2-yl)-1H-pyrazol-1-yl)piperidin-1-yl)propyl)amino)pyrimidin-4-yl)amino)isoindolin-2-yl)piperidine-2,6-dione (GS-085)

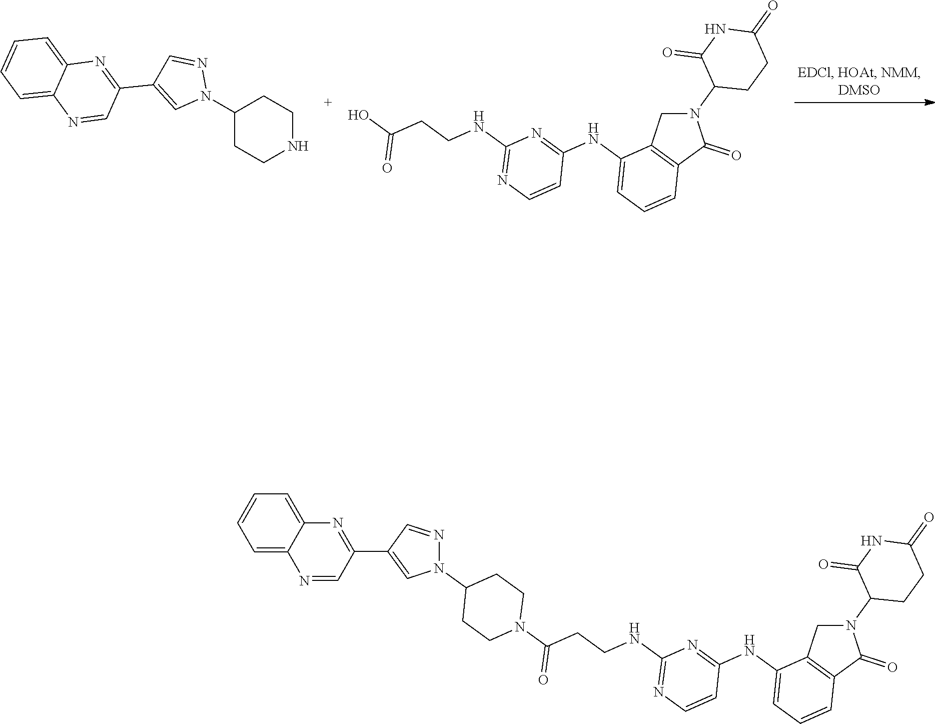 [SMILES: c1ccc2nc(-c3cnn(C4CCNCC4)c3)cnc2c1 ; O=C(O)CCNc1nccc(Nc2cccc3c2CN(C2CCC(=O)NC2=O)C3=O)n1 ; O=C1CCC(N2Cc3c(Nc4ccnc(NCCC(=O)N5CCC(n6cc(-c7cnc8ccccc8n7)cn6)CC5)n4)cccc3C2=O)C(=O)N1]

GS-085 was synthesized following the similar procedure for preparing GS-001 (1.0 mg, yield: 10.3%). MS (ESI) m/z=686.7 [M+H]⁺.

### Example 44. N-((2-(2,6-dioxopiperidin-3-yl)-1-oxoisoindolin-5-yl)methyl)-2-(3-(4-(4-(quinoxalin-2-yl)-1H-pyrazol-1-yl)piperidin-1-yl)cyclohexyl)acetamide (GS-086)

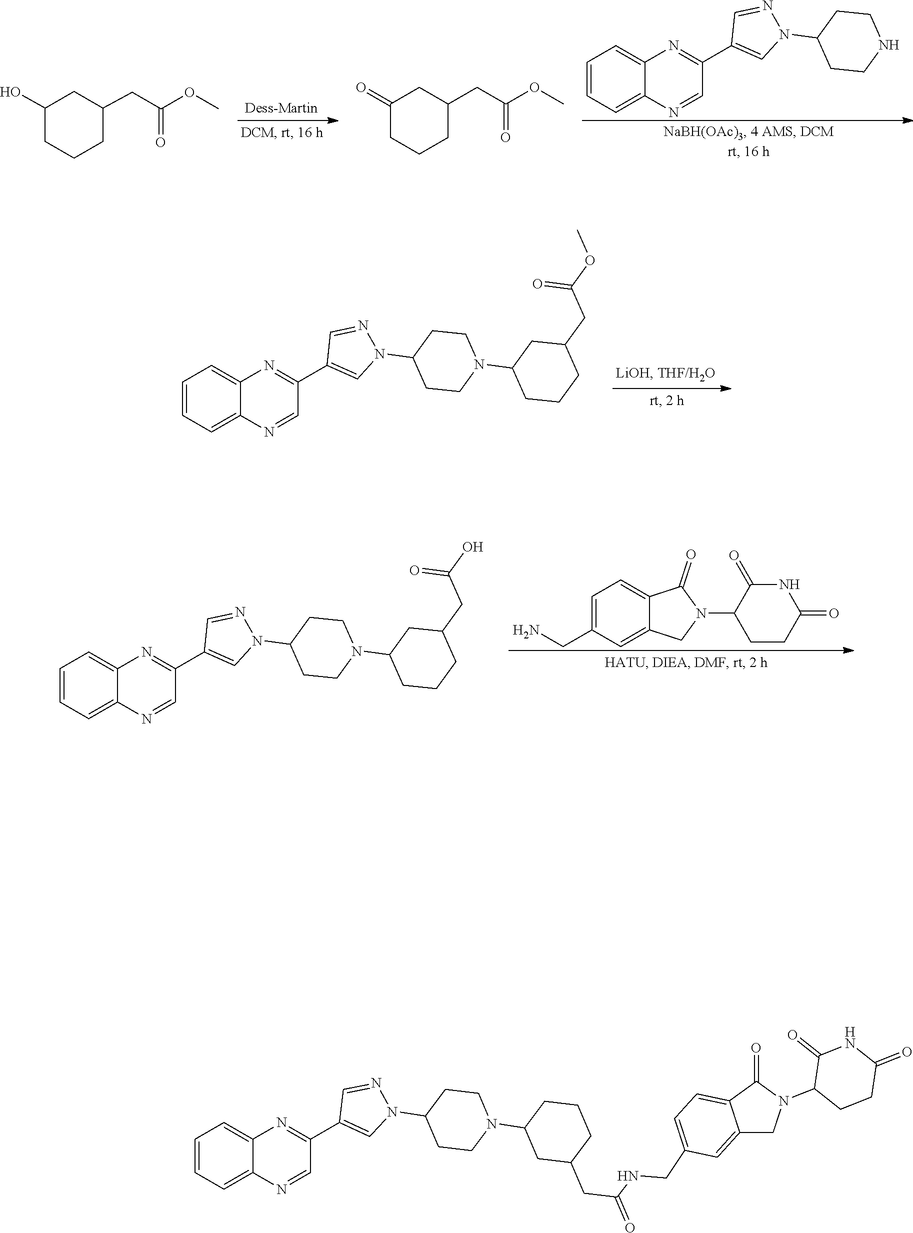

Step 1: Synthesis of methyl 2-(3-oxocyclohexyl)acetate

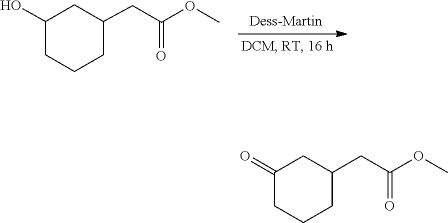

To a stirred solution of methyl 2-(3-hydroxycyclohexyl)acetate (300 mg, 0.17 mmol) in DCM (6.0 mL) was added Dess-Martin Periodinane (1.5 g, 3.48 mmol). The reaction was stirred at rt for 16 h, before it was filtered. The filtrate was concentrated. And The resulting residue was purified by column chromatography (EtOAc:petroleum ether=1:3) to provide the title compound (265 mg, 76.6% yield) as light-yellow oil which was used directly in the next step.

Step 2: Synthesis of methyl 2-(3-(4-(4-(quinoxalin-2-yl)-1H-pyrazol-1-yl)piperidin-1-yl)cyclohexyl)acetate

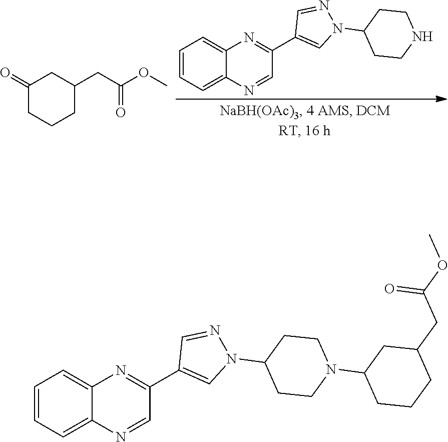

To a stirred solution of methyl 2-(3-oxocyclohexyl)acetate (61 mg, 0.36 mmol) and 2-(1-(piperidin-4-yl)-1H-pyrazol-4-yl)quinoxaline (50 mg, 0.18 mmol) in DCM (2.0 mL) were added activated molecular sieves (50 mg) and NaBH(OAc)₃ (114 mg, 0.54 mmol). The reaction was stirred at rt under N₂ for 16 h. The reaction mixture was filtered, and the filtrate was concentrated. The resulting residue was purified by prep-TLC (DCM:MeOH=10:1) to provide the title compound (12 mg, 15.4% yield) as solid. MS (ESI) m/z=434.4 [M+H]⁺.

Step 3: Synthesis of 2-(3-(4-(4-(quinoxalin-2-yl)-1H-pyrazol-1-yl)piperidin-1-yl)cyclohexyl)acetic acid

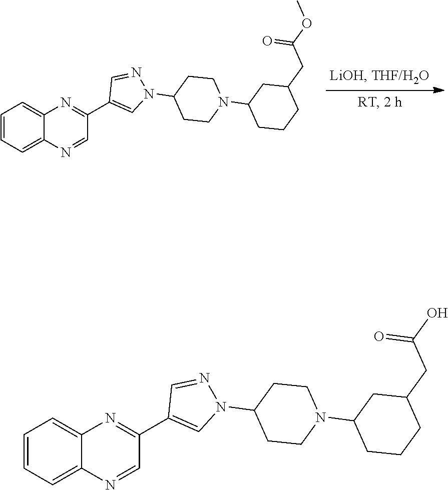 [SMILES: COC(=O)CC1CCCC(N2CCC(n3cc(-c4cnc5ccccc5n4)cn3)CC2)C1 ; O=C(O)CC1CCCC(N2CCC(n3cc(-c4cnc5ccccc5n4)cn3)CC2)C1]

To a solution of methyl 2-(3-(4-(4-(quinoxalin-2-yl)-1H-pyrazol-1-yl)piperidin-1-yl)cyclohexyl)acetate (12 mg, 0.028 mmol) in THF (1.0 mL) and H₂O (1.0 mL) was added LiOH H₂O (7.0 mg, 0.166 mmol). The reaction was stirred at rt for 2 h. The pH of the reaction solution was adjusted to 2˜3 with HCl (1M). The mixture was extracted with EtOAc and washed with water and brine. The organic layer was dried over Na₂SO₄, filtered, and concentrated to provide the crude product (11 mg, 94.8% yield) which was used directly in the next step. MS (ESI) m/z=420.5 [M+H]⁺.

Step 4: Synthesis of N-((2-(2,6-dioxopiperidin-3-yl)-1-oxoisoindolin-5-yl)methyl)-2-(3-(4-(4-(quinoxalin-2-yl)-1H-pyrazol-1-yl)piperidin-1-yl)cyclohexyl)acetamide

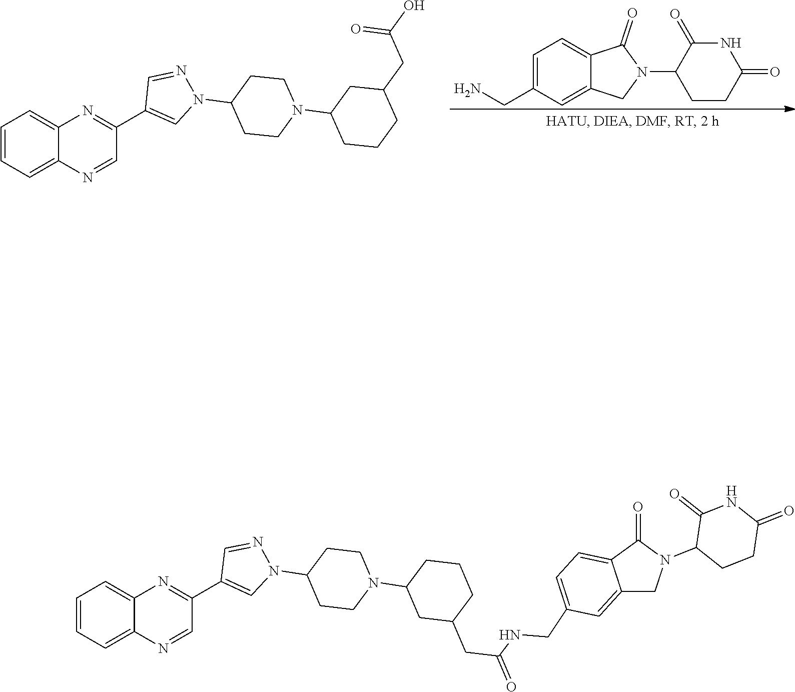 [SMILES: O=C(O)CC1CCCC(N2CCC(n3cc(-c4cnc5ccccc5n4)cn3)CC2)C1 ; NCc1ccc2c(c1)CN(C1CCC(=O)NC1=O)C2=O ; O=C(CC1CCCC(N2CCC(n3cc(-c4cnc5ccccc5n4)cn3)CC2)C1)NCc1ccc2c(c1)CN(C1CCC(=O)NC1=O)C2=O]

To a solution of 2-(3-(4-(4-(quinoxalin-2-yl)-1H-pyrazol-1-yl)piperidin-1-yl)cyclohexyl)acetic acid (11 mg, 0.026 mmol) in DMF (2.0 mL) were added DIEA (17 mg, 0.131 mmol), HATU (15 mg, 0.0394 mmol) and 3-(5-(aminomethyl)-1-oxoisoindolin-2-yl)piperidine-2,6-dione hydrochloride (10 mg, 0.031 mmol). The reaction was stirred at rt for 2 h. The reaction mixture was concentrated. The resulting residue was purified by prep-TLC (DCM:MeOH=5:1) to provide the title compound (16 mg, 90% yield) as a whit solid. MS (ESI) m/z=675.7 [M+H]⁺.

### Example 45. N-((5-(2,6-dioxopiperidin-3-yl)-4-oxo-5,6-dihydro-4H-thieno[3,4-c]pyrrol-1-yl)methyl)-2-((3-(4-(4-(quinoxalin-2-yl)-1H-pyrazol-1-yl)piperidin-1-yl)phenyl)amino)acetamide (GS-087)

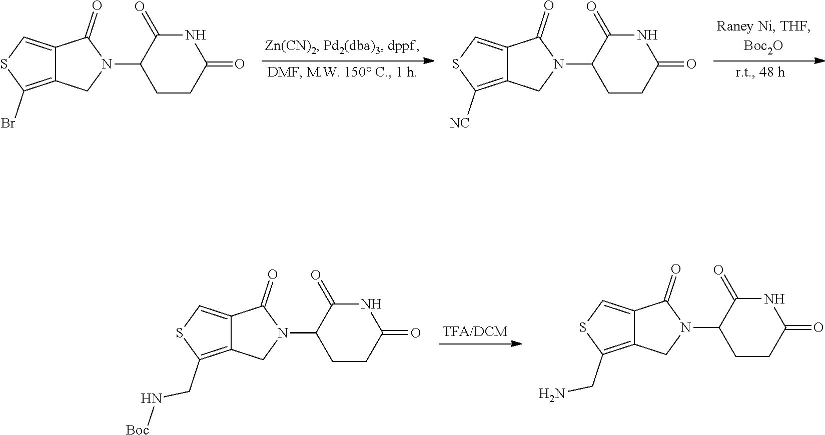 [SMILES: O=C1CCC(N2Cc3c(csc3Br)C2=O)C(=O)N1 ; N#Cc1scc2c1CN(C1CCC(=O)NC1=O)C2=O ; CC(C)(C)OC(=O)NCc1scc2c1CN(C1CCC(=O)NC1=O)C2=O ; NCc1scc2c1CN(C1CCC(=O)NC1=O)C2=O]

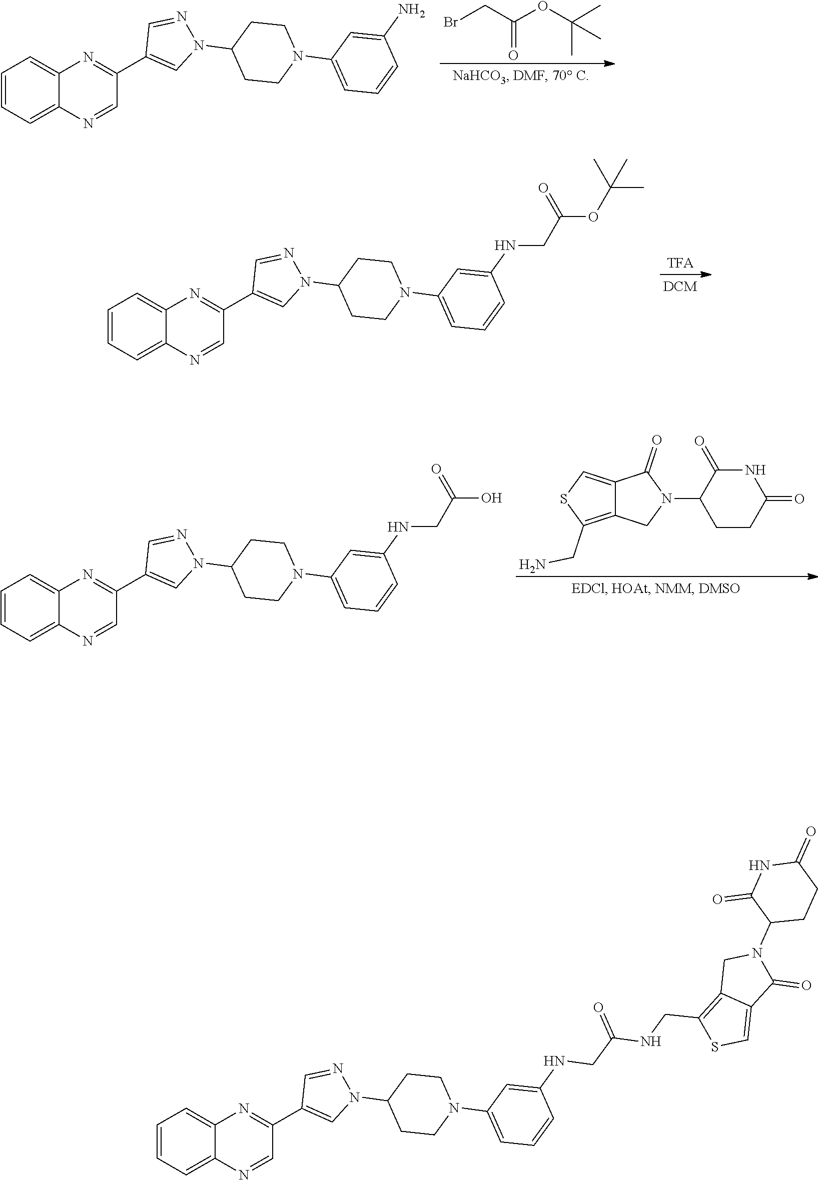

Step 1: Synthesis of 5-(2,6-dioxopiperidin-3-yl)-4-oxo-5,6-dihydro-4H-thieno[3,4-c]pyrrole-1-carbonitrile

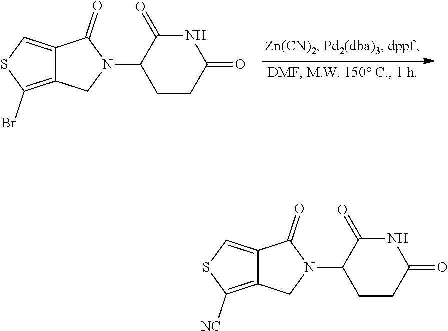 [SMILES: O=C1CCC(N2Cc3c(csc3Br)C2=O)C(=O)N1 ; N#Cc1scc2c1CN(C1CCC(=O)NC1=O)C2=O]

A solution of 3-(1-bromo-4-oxo-4H-thieno[3,4-c]pyrrol-5(6H)-yl)piperidine-2,6-dione (500 mg, 1.52 mmol), Pd₂(dba)₃ (139 mg, 0.152 mmol), Zn(CN)₂ (534 mg, 4.56 mmol) and DPPF (84 mg, 0.152 mmol) in DMF (6 mL) was irradiated at 150° C. for 1 h under microwave irradiation reactor under argon atmosphere. After being cooled to rt, the mixture was concentrated. The residue was purified by silica gel column chromatography (PE:EtOAc=1:1) to provide the title compound (300 mg, 71.7% yield) as an off-yellow solid. MS (ESI) m/z=274.0 [M−H]⁻.

Step 2: Synthesis of tert-butyl ((5-(2,6-dioxopiperidin-3-yl)-4-oxo-5,6-dihydro-4H-thieno[3,4-c]pyrrol-1-yl)methyl)carbamate

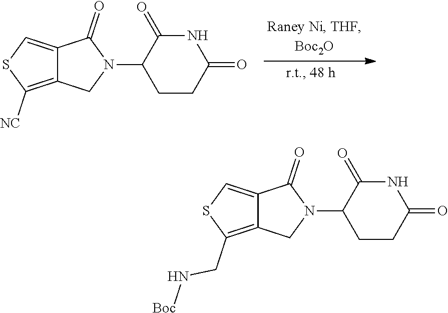 [SMILES: N#Cc1scc2c1CN(C1CCC(=O)NC1=O)C2=O ; CC(C)(C)OC(=O)NCc1scc2c1CN(C1CCC(=O)NC1=O)C2=O]

A solution of methyl 5-(2,6-dioxopiperidin-3-yl)-4-oxo-5,6-dihydro-4H-thieno[3,4-c]pyrrole-1-carbonitrile (300 mg, 1.09 mmol), Raney Ni (100 mg) and Boc₂O (594 mg, 2.73 mmol) in THF (20 mL) was stirred at rt for 48 h under hydrogen atmosphere (15 psi). The mixture was filtered, and the filtrate was concentrated in vacuum to provide a residue, which was purified by silica gel column chromatography (petroleum ether:EtOAc=1:1) to provide the title compound (60 mg, 14.5% yield) as an off-yellow solid. ¹H NMR (400 MHz, DMSO-d₆): δ 10.99 (s, 1H), 7.86 (s, 1H), 7.55 (t, J=6.4 Hz, 1H), 5.02 (dd, J=13.2 Hz, 4.8 Hz, 1H), 4.29-4.14 (m, 4H), 2.89-2.84 (m, 1H), 2.73-2.57 (m, 1H), 2.32-2.26 (m, 1H), 2.07-1.95 (m, 1H), 1.46 (s, 9H). MS (ESI) m/z=324.1 [M+H]⁺.

Step 3: Synthesis of 3-(1-(aminomethyl)-4-oxo-4H-thieno[3,4-c]pyrrol-5(6H)-yl)piperidine-2,6-dione

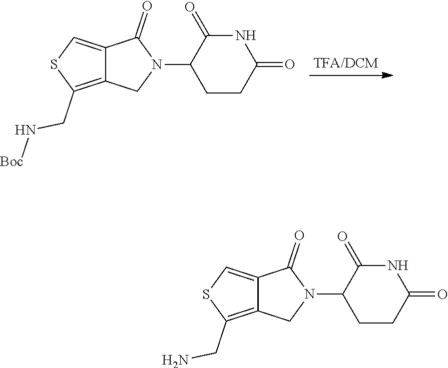 [SMILES: CC(C)(C)OC(=O)NCc1scc2c1CN(C1CCC(=O)NC1=O)C2=O ; NCc1scc2c1CN(C1CCC(=O)NC1=O)C2=O]

To a solution of tert-butyl ((5-(2,6-dioxopiperidin-3-yl)-4-oxo-5,6-dihydro-4H-thieno[3,4-c]pyrrol-1-yl)methyl)carbamate (380 mg, 1.0 mmol) in DCM (3.0 mL) was added TFA (1.5 mL). After the reaction mixture was stirred at rt for 2 h, it was concentrated to provide the crude title compound which was used directly in the next step without further purification.

Step 4: Synthesis of tert-butyl (3-(4-(4-(quinoxalin-2-yl)-1H-pyrazol-1-yl)piperidin-1-yl)phenyl)glycinate

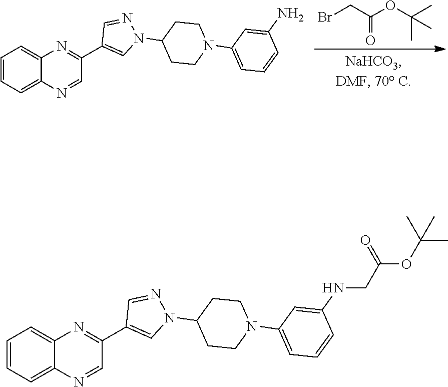

A mixture of 3-[4-(4-quinoxalin-2-ylpyrazol-1-yl)-1-piperidyl]aniline (100 mg, 0.27 mmol), tert-butyl 2-bromoacetate (63 mg, 0.32 mmol) and NaHCO₃ (45 mg, 0.54 mmol) in DMF (2 mL) was stirred at 70° C. for 12 h. The mixture was purified by reverse phase column chromatography to provide the title compound (50 mg, 38.2% yield) as a yellow solid. MS (ESI) m/z=485.6 [M+H]⁺.

Step 5: Synthesis of (3-(4-(4-(quinoxalin-2-yl)-1H-pyrazol-1-yl)piperidin-1-yl)phenyl)glycine

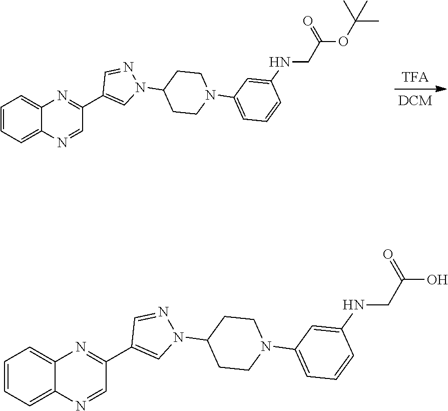 [SMILES: CC(C)(C)OC(=O)CNc1cccc(N2CCC(n3cc(-c4cnc5ccccc5n4)cn3)CC2)c1 ; O=C(O)CNc1cccc(N2CCC(n3cc(-c4cnc5ccccc5n4)cn3)CC2)c1]

A mixture of tert-butyl 2-[3-[4-(4-quinoxalin-2-ylpyrazol-1-yl)-1-piperidyl]anilino]acetate (50 mg, 0.10 mmol) and TFA (2 mL) in DCM (2 mL) was stirred at 25° C. for 1 h. The reaction was concentrated to provide the title compound (30 mg, 67.9% yield) as a yellow solid. MS (ESI) m/z=429.4 [M+H]⁺.

Step 6: Synthesis of N-((5-(2,6-dioxopiperidin-3-yl)-4-oxo-5,6-dihydro-4H-thieno[3,4-c]pyrrol-1-yl)methyl)-2-((3-(4-(4-(quinoxalin-2-yl)-1H-pyrazol-1-yl)piperidin-1-yl)phenyl)amino)acetamide

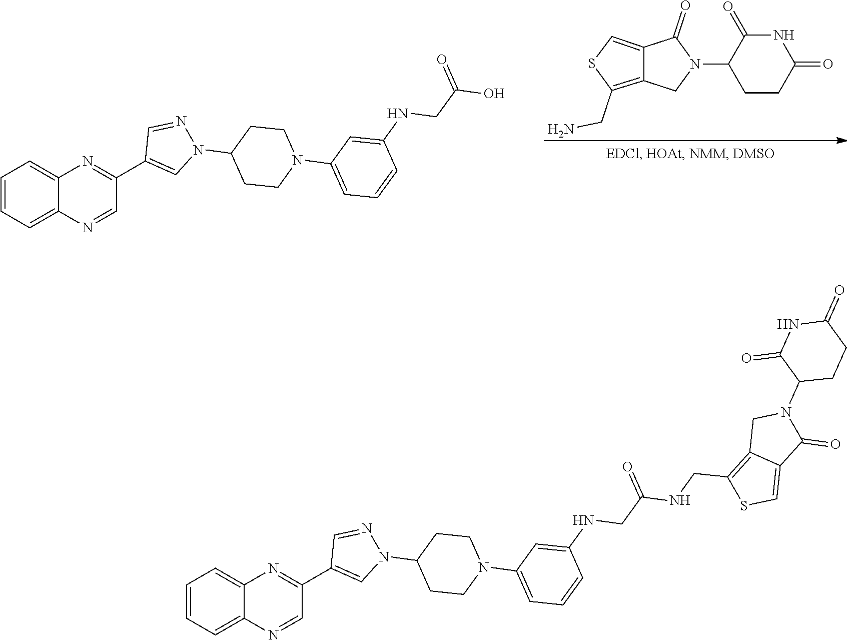 [SMILES: O=C(O)CNc1cccc(N2CCC(n3cc(-c4cnc5ccccc5n4)cn3)CC2)c1 ; NCc1scc2c1CN(C1CCC(=O)NC1=O)C2=O ; O=C(CNc1cccc(N2CCC(n3cc(-c4cnc5ccccc5n4)cn3)CC2)c1)NCc1scc2c1CN(C1CCC(=O)NC1=O)C2=O]

To a solution of 2-[3-[4-(4-quinoxalin-2-ylpyrazol-1-yl)-1-piperidyl]anilino]acetic acid (12 mg, 0.027 mol) in DMSO (0.5 mL) were added HOAt (7.3 mg, 0.054 mol), EDCI (10.3 mg, 0.054 mmol) and NMM (9.1 mg, 0.09 mol). The mixture was stirred at rt for 2 min, before 3-[3-(aminomcthyl)-6-oxo-4H-thieno[3,4-c]pyrrol-5-yl]piperidine-2,6-dione (5 mg, 0.018 mmol) was added. After the reaction was stirred at 25° C. for 12 h, the mixture was purified by reverse phase column chromatography to provide the title compound (4.7 mg, 38.1% yield). MS (ESI) m/z=690.6 [M+H]⁺.

### Example 46. 3-(1-Oxo-4-((2-((3-(4-(4-(quinoxalin-2-yl)-1H-pyrazol-1-yl)piperidin-1-yl)propyl)amino)pyrimidin-4-yl)amino)isoindolin-2-yl)piperidine-2,6-dione (GS-088)

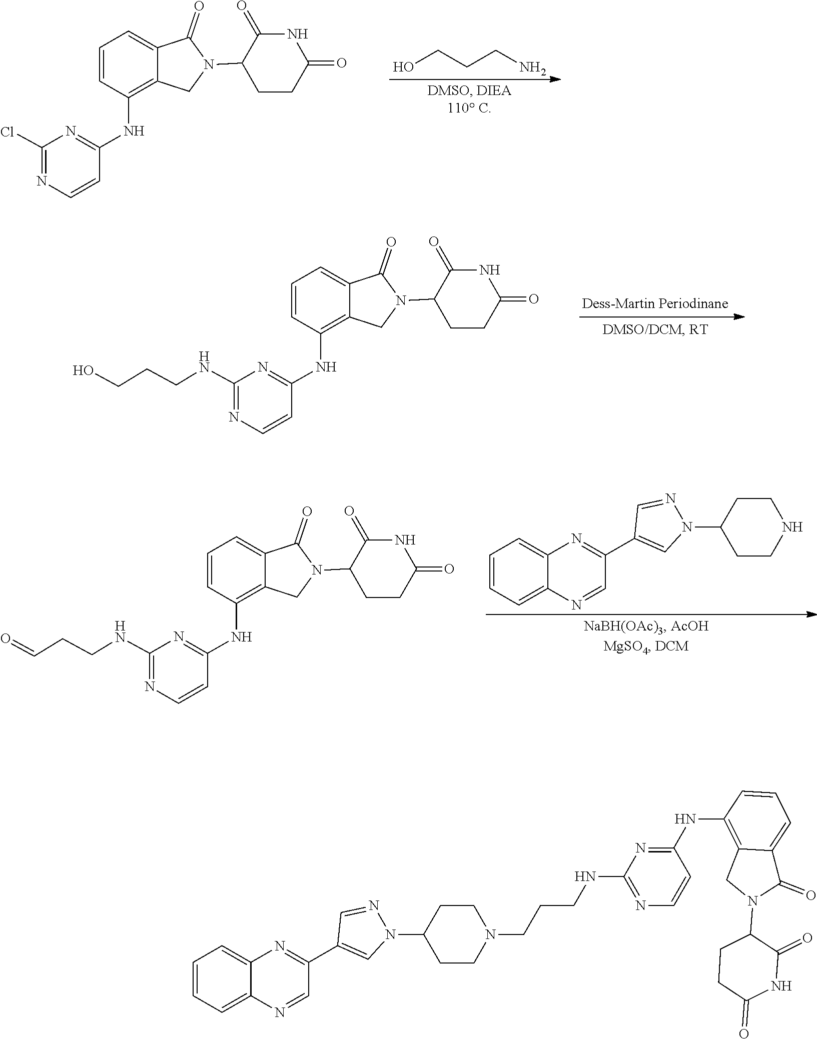

Step 1: Synthesis of 3-(4-((2-((3-hydroxypropyl)amino)pyrimidin-4-yl)amino)-1-oxoisoindolin-2-yl)piperidine-2,6-dione

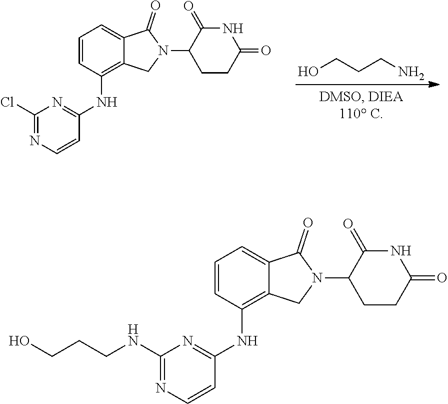 [SMILES: O=C1CCC(N2Cc3c(Nc4ccnc(Cl)n4)cccc3C2=O)C(=O)N1 ; NCCCO ; O=C1CCC(N2Cc3c(Nc4ccnc(NCCCO)n4)cccc3C2=O)C(=O)N1]

To a stirred solution of 3-(4-((2-chloropyrimidin-4-yl)amino)-1-oxoisoindolin-2-yl)piperidine-2,6-dione (100 mg, 0.269 mmol) and 3-aminopropan-1-ol (40.3 mg, 0.538 mmol) in DMSO (5 mL) was added DIEA (174 mg, 1.35 mmol). After the mixture was stirred at 90° C. overnight, it was diluted with H₂O (100 mL) and extracted with ethyl acetate (100 mL×3). The organic layer was washed with brine, dried over Na₂SO₄ and concentrated. The resulting residue was purified by flash chromatography to provide the title compound (760 mg, 42.4% yield) as a yellow solid.

Step 2: Synthesis of 3-((4-((2-(2,6-dioxopiperidin-3-yl)-1-oxoisoindolin-4-yl)amino)pyrimidin-2-yl)amino)propanal

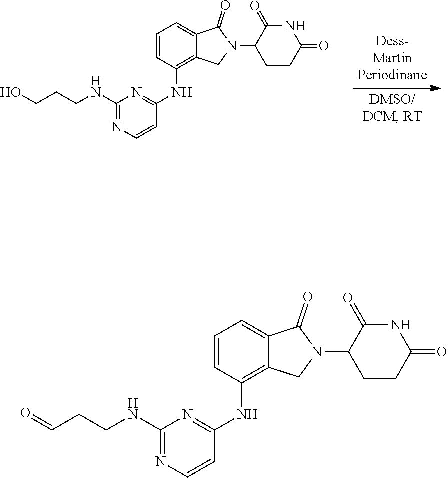 [SMILES: O=C1CCC(N2Cc3c(Nc4ccnc(NCCCO)n4)cccc3C2=O)C(=O)N1 ; O=CCCNc1nccc(Nc2cccc3c2CN(C2CCC(=O)NC2=O)C3=O)n1]

To a stirred solution of 3-(4-((2-((3-hydroxypropyl)amino)pyrimidin-4-yl)amino)-1-oxoisoindolin-2-yl)piperidine-2,6-dione (80 mg, 0.195 mmol) in DMSO (5 mL) and DCM (5 mL) was added Dess-Martin periodinane (165 mg, 0.39 mmol). The reaction mixture was stirred at rt for 1.5 h, before it was concentrated and purified by prep-HPLC to provide the title compound (65 mg, 82.3% yield) as a white solid. MS (ESI) m/z=409.4 [M+H]⁺.

Step 3: Synthesis of 3-(1-oxo-4-((2-((3-(4-(4-(quinoxalin-2-yl)-1H-pyrazol-1-yl)piperidin-1-yl)propyl)amino)pyrimidin-4-yl)amino)isoindolin-2-yl)piperidine-2,6-dione

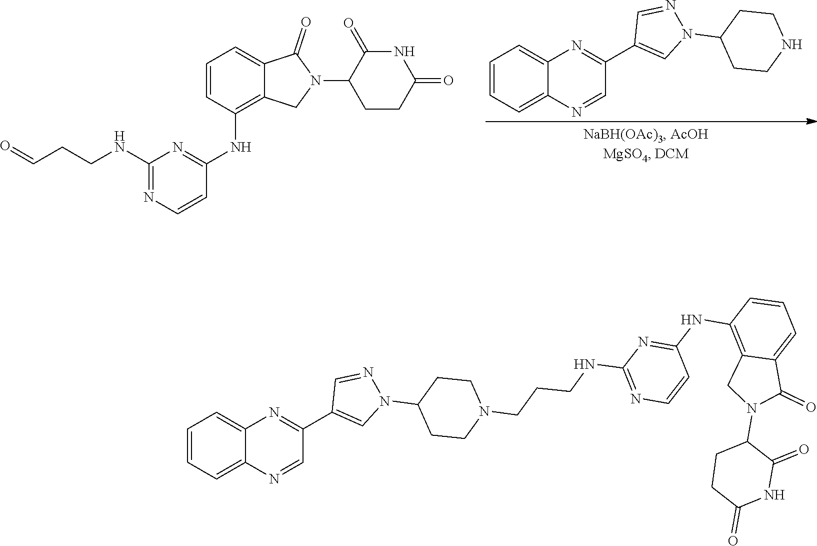

To a stirred solution of 2-(1-(piperidin-4-yl)-1H-pyrazol-4-yl)quinoxaline 7.28 mg, 0.026 mmol) and 3-((4-((2-(2,6-dioxopiperidin-3-yl)-1-oxoisoindolin-4-yl)amino)pyrimidin-2-yl)amino)propanal (10.0 mg, 0.026 mmol) in DCM (5 mL) were added MgSO₄ (50 mg, 0.416 mmol) and NaBH(OAc)₃ (16.5 mg, 0.078 mmol). The reaction mixture was stirred at 40° C. for 3 h, before it was quenched with H₂O. The mixture was extracted with DCM (20 mL×2), washed with brine, dried over Na₂SO₄ and concentrated.

The resulting residue was purified by prep-HPLC, followed by prep-TLC to provide the title compound (4.3 mg, 25.3% yield) as a yellow solid. MS (ESI) m/z=672.5 [M+H]⁺.

### Example 47. N-((2-(2,6-dioxopiperidin-3-yl)-1-oxoisoindolin-5-yl)methyl)-2-(4-(4-(4-(quinoxalin-2-yl)-1H-pyrazol-1-yl)piperidin-1-yl)cyclohexyl)acetamide (GS-089)

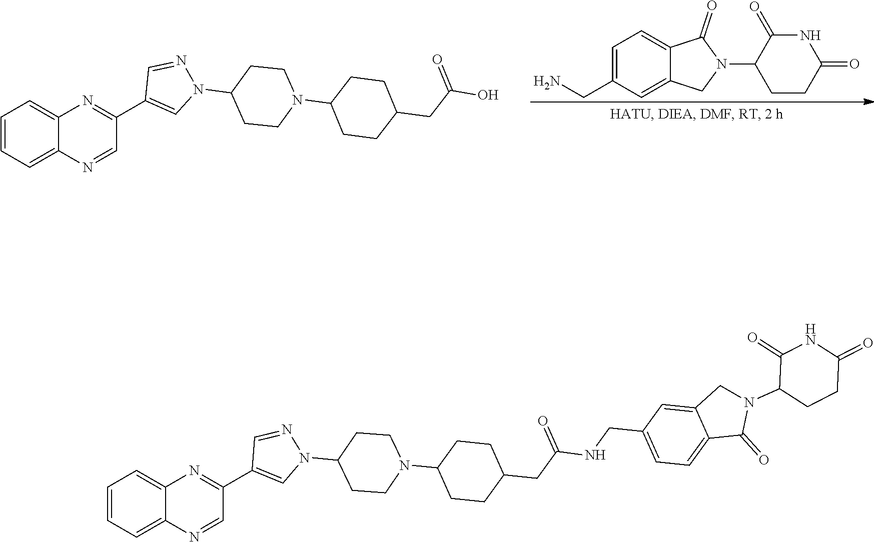 [SMILES: O=C(O)CC1CCC(N2CCC(n3cc(-c4cnc5ccccc5n4)cn3)CC2)CC1 ; NCc1ccc2c(c1)CN(C1CCC(=O)NC1=O)C2=O ; O=C(CC1CCC(N2CCC(n3cc(-c4cnc5ccccc5n4)cn3)CC2)CC1)NCc1ccc2c(c1)CN(C1CCC(=O)NC1=O)C2=O]

GS-089 was synthesized following the similar procedure for preparing GS-086 (18 mg, yield: 93.3%). MS (ESI) m/z=675.6 [M+H]⁺.

### Example 48. 3-(1-Oxo-4-((6-((3-(4-(4-(quinoxalin-2-yl)-1H-pyrazol-1-yl)piperidin-1-yl)phenyl)amino)pyridin-2-yl)amino)isoindolin-2-yl)piperidine-2,6-dione (GS-090)

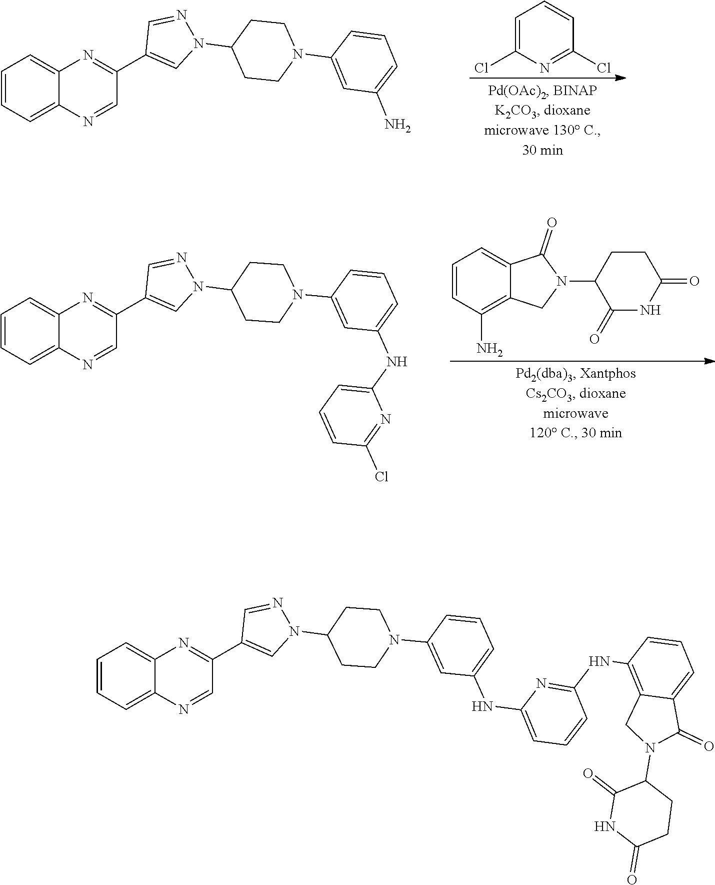 [SMILES: Nc1cccc(N2CCC(n3cc(-c4cnc5ccccc5n4)cn3)CC2)c1 ; Clc1cccc(Cl)n1 ; Clc1cccc(Nc2cccc(N3CCC(n4cc(-c5cnc6ccccc6n5)cn4)CC3)c2)n1 ; Nc1cccc2c1CN(C1CCC(=O)NC1=O)C2=O ; O=C1CCC(N2Cc3c(Nc4cccc(Nc5cccc(N6CCC(n7cc(-c8cnc9ccccc9n8)cn7)CC6)c5)n4)cccc3C2=O)C(=O)N1]

Step 1: Synthesis of 6-chloro-N-(3-(4-(4-(quinoxalin-2-yl)-1H-pyrazol-1-yl)piperidin-1-yl)phenyl)pyridin-2-amine

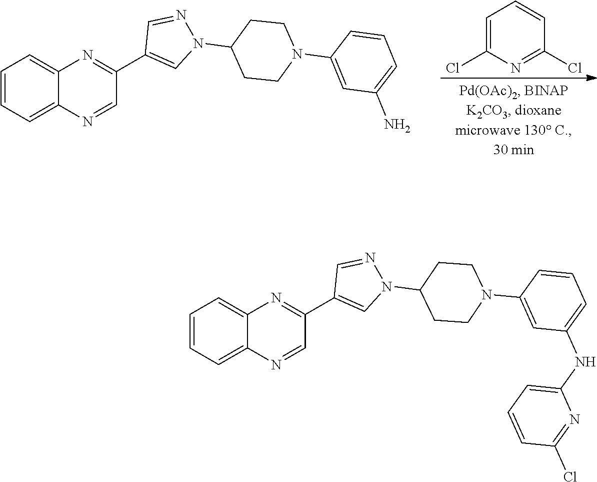 [SMILES: Nc1cccc(N2CCC(n3cc(-c4cnc5ccccc5n4)cn3)CC2)c1 ; Clc1cccc(Cl)n1 ; Clc1cccc(Nc2cccc(N3CCC(n4cc(-c5cnc6ccccc6n5)cn4)CC3)c2)n1]

To a solution of 3-(4-(4-(quinoxalin-2-yl)-1H-pyrazol-1-yl)piperidin-1-yl)aniline (67.5 mg, 0.182 mmol) and 2,6-dichloropyridine (20 mg, 0.135 mmol) in 1,4-dioxane (2.0 mL) were added BINAP (1.9 mg, 0.003 mmol), Pd₂(dba)₃ (0.68 mg, 0.003 mmol) and K₂CO₃ (74 mg, 0.536 mmol). The reaction was stirred irradiated at 130° C. under microwave irradiation reactor for 30 min, before it was concentrated and purified by prep-TLC (EtOAc:petroleum ether=1:1) to provide the title compound (54.5 mg, 84% yield) as a yellow solid. MS (ESI) m/z=482.4 [M+H]⁺.

Step 2: Synthesis of 3-(1-oxo-4-((6-((3-(4-(4-(quinoxalin-2-yl)-1H-pyrazol-1-yl)piperidin-1-yl)phenyl)amino)pyridin-2-yl)amino)isoindolin-2-yl)piperidine-2,6-dione

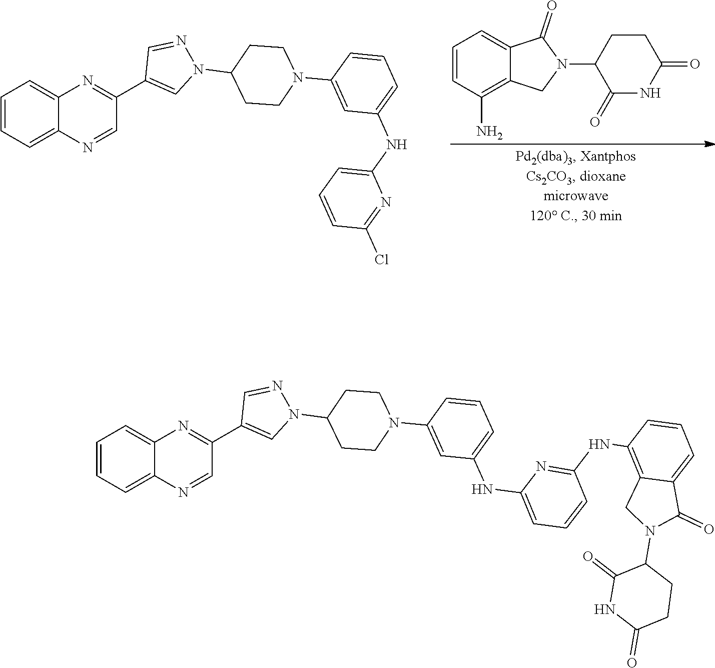 [SMILES: Clc1cccc(Nc2cccc(N3CCC(n4cc(-c5cnc6ccccc6n5)cn4)CC3)c2)n1 ; Nc1cccc2c1CN(C1CCC(=O)NC1=O)C2=O ; O=C1CCC(N2Cc3c(Nc4cccc(Nc5cccc(N6CCC(n7cc(-c8cnc9ccccc9n8)cn7)CC6)c5)n4)cccc3C2=O)C(=O)N1]

To a solution of 6-chloro-N-(3-(4-(4-(quinoxalin-2-yl)-1H-pyrazol-1-yl)piperidin-1-yl)phenyl)pyridin-2-amine (22 mg, 0.046 mmol) and 3-(4-amino-1-oxoisoindolin-2-yl)piperidine-2,6-dione (17.8 mg, 0.069 mmol) in 1,4-dioxane (1.0 mL) were added Pd₂(dba)₃ (0.68 mg, 0.003 mmol), Xantphos (5.3 mg, 0.009 mmol) and Cs₂CO₃ (30 mg, 0.035 mmol). The reaction was irradiated at 125° C. under microwave irradiation reactor for 30 min, before it was concentrated and purified by prep-TLC (DCM:MeOH=10:1) to provide the title compound (11.6 mg, 36% yield) as a yellow solid. MS (ESI) m/z=705.5 [M+H]⁺.

### Example 49. 3-(1-Oxo-4-((2-((2-oxo-2-(4-(4-(quinoxalin-2-yl)-1H-pyrazol-1-yl)piperidin-1-yl)ethyl)amino)pyrimidin-4-yl)amino)isoindolin-2-yl)piperidine-2,6-dione (GS-091)

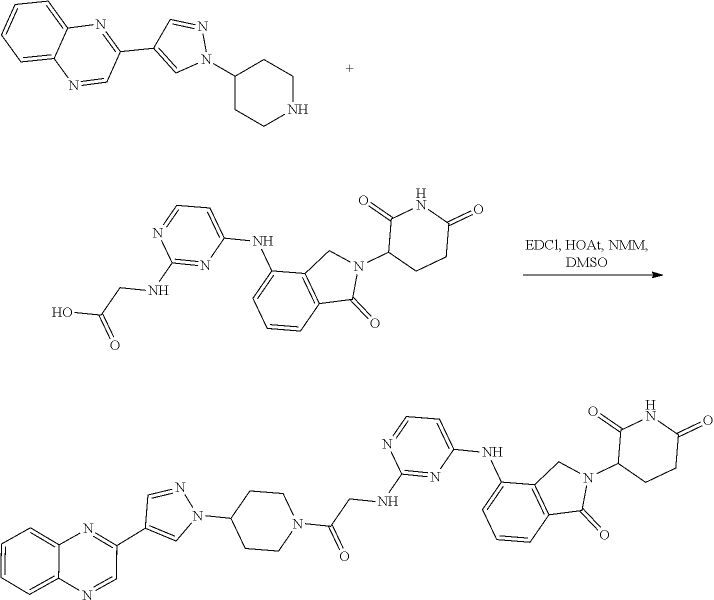 [SMILES: c1ccc2nc(-c3cnn(C4CCNCC4)c3)cnc2c1 ; O=C(O)CNc1nccc(Nc2cccc3c2CN(C2CCC(=O)NC2=O)C3=O)n1 ; O=C1CCC(N2Cc3c(Nc4ccnc(NCC(=O)N5CCC(n6cc(-c7cnc8ccccc8n7)cn6)CC5)n4)cccc3C2=O)C(=O)N1]

GS-091 was synthesized following the standard procedure for preparing GS-001 (2.1 mg, yield: 16%). MS (ESI) m/z=672.6 [M+H]⁺.

### Example 50. 3-(1-Oxo-4-((2-((3-(4-(4-(quinoxalin-2-yl)-1H-pyrazol-1-yl)piperidin-1-yl)cyclopentyl)amino)pyrimidin-4-yl)amino)isoindolin-2-yl)piperidine-2,6-dione (GS-092)

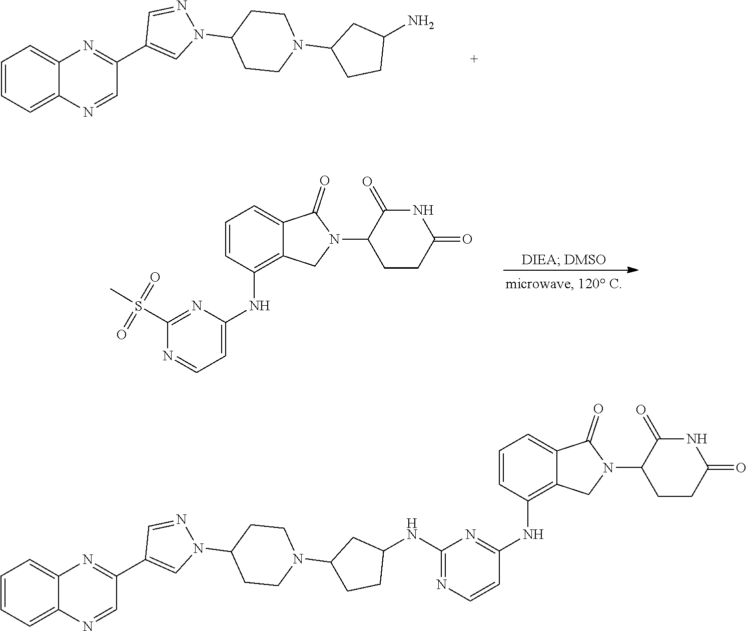 [SMILES: NC1CCC(N2CCC(n3cc(-c4cnc5ccccc5n4)cn3)CC2)C1 ; CS(=O)(=O)c1nccc(Nc2cccc3c2CN(C2CCC(=O)NC2=O)C3=O)n1 ; O=C1CCC(N2Cc3c(Nc4ccnc(NC5CCC(N6CCC(n7cc(-c8cnc9ccccc9n8)cn7)CC6)C5)n4)cccc3C2=O)C(=O)N1]

GS-092 was synthesized following the similar procedure for preparing GS-074 (10 mg, yield: 12%). MS (ESI) m/z=698.6 [M+H]⁺.

### Example 51. 1-((5-(2,6-Dioxopiperidin-3-yl)-4-oxo-5,6-dihydro-4H-thieno[3,4-c]pyrrol-1-yl)methyl)-3-(3-(4-(4-(quinoxalin-2-yl)-1H-pyrazol-1-yl)piperidin-1-yl)phenyl)urea (GS-093)

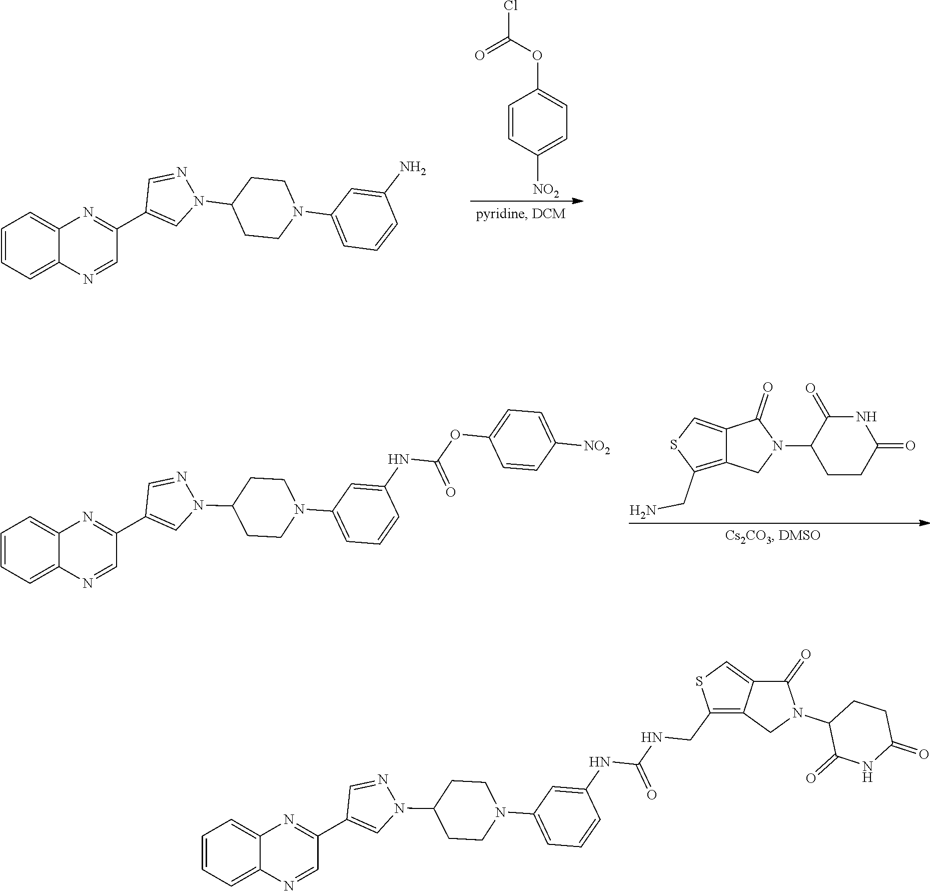 [SMILES: Nc1cccc(N2CCC(n3cc(-c4cnc5ccccc5n4)cn3)CC2)c1 ; O=C(Cl)Oc1ccc([N+](=O)[O-])cc1 ; O=C(Nc1cccc(N2CCC(n3cc(-c4cnc5ccccc5n4)cn3)CC2)c1)Oc1ccc([N+](=O)[O-])cc1 ; NCc1scc2c1CN(C1CCC(=O)NC1=O)C2=O ; O=C1CCC(N2Cc3c(csc3CNC(=O)Nc3cccc(N4CCC(n5cc(-c6cnc7ccccc7n6)cn5)CC4)c3)C2=O)C(=O)N1]

Step 1: Synthesis of 4-nitrophenyl (3-(4-(4-(quinoxalin-2-yl)-1H-pyrazol-1-yl)piperidin-1-yl)phenyl)carbamate

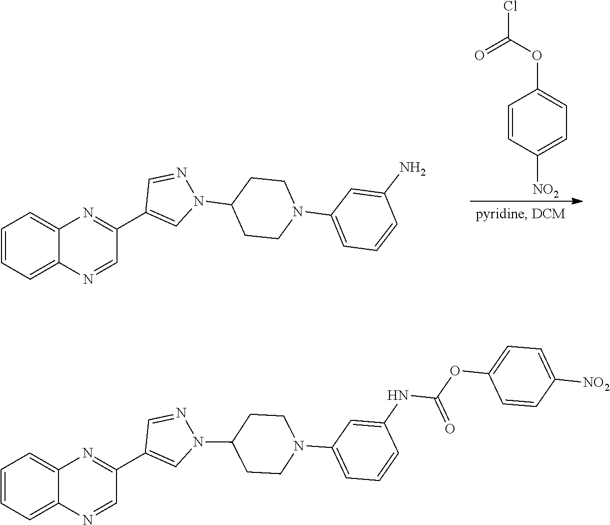 [SMILES: Nc1cccc(N2CCC(n3cc(-c4cnc5ccccc5n4)cn3)CC2)c1 ; O=C(Cl)Oc1ccc([N+](=O)[O-])cc1 ; O=C(Nc1cccc(N2CCC(n3cc(-c4cnc5ccccc5n4)cn3)CC2)c1)Oc1ccc([N+](=O)[O-])cc1]

To a stirred solution of 3-(4-(4-(quinoxalin-2-yl)-1H-pyrazol-1-yl)piperidin-1-yl)aniline (25 mg, 0.068 mmol) and pyridine (16 mg, 0.204 mmol) in anhydrous THF (2.5 mL) was added (4-nitrophenyl) carbonochloridate (11 mg, 0.054 mmol) at rt. After the mixture was stirred for 2 h. The mixture was used directly in the next step without further operation. MS (ESI) m/z=536.4 [M+H]⁺.

Step 2: Synthesis of 1-((5-(2,6-dioxopiperidin-3-yl)-4-oxo-5,6-dihydro-4H-thieno[3,4-c]pyrrol-1-yl)methyl)-3-(3-(4-(4-(quinoxalin-2-yl)-1H-pyrazol-1-yl)piperidin-1-yl)phenyl)urea

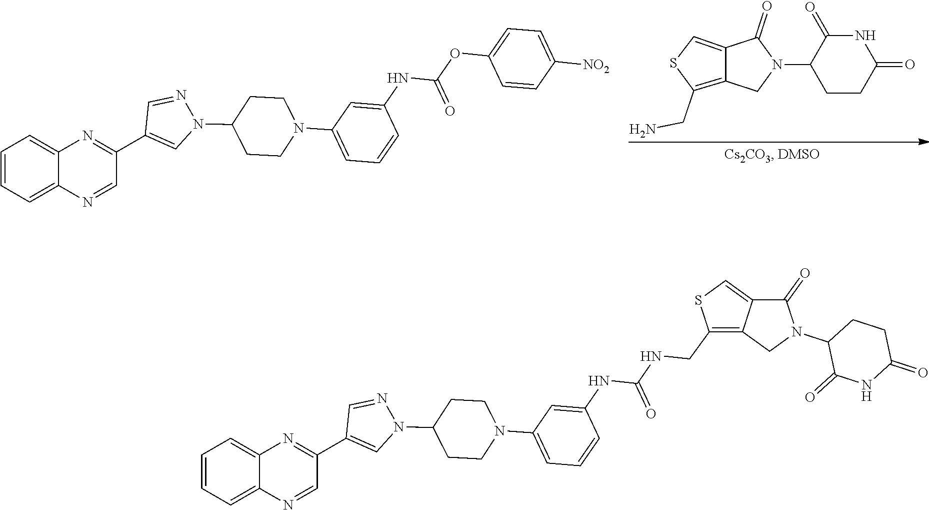 [SMILES: O=C(Nc1cccc(N2CCC(n3cc(-c4cnc5ccccc5n4)cn3)CC2)c1)Oc1ccc([N+](=O)[O-])cc1 ; NCc1scc2c1CN(C1CCC(=O)NC1=O)C2=O ; O=C1CCC(N2Cc3c(csc3CNC(=O)Nc3cccc(N4CCC(n5cc(-c6cnc7ccccc7n6)cn5)CC4)c3)C2=O)C(=O)N1]

To a stirred solution 3-(4-(4-(quinoxalin-2-yl)-1H-pyrazol-1-yl)piperidin-1-yl)phenyl)carbamate (29 mg, 0.054 mmol) in anhydrous DMF (0.5 mL) and THF (2.5 mL) were added 3-(1-(aminomethyl)-4-oxo-4H-thieno[3,4-c]pyrrol-5(6H)-yl)piperidine-2,6-dione (15 mg, 0.054 mmol) and Cs₂CO₃ (26 mg, 0.081 mol). The resulting mixture was stirred at 60° C. for 2 h. The mixture was purified by reverse phase column chromatography, followed by prep-TLC (DCM:MeOH=10:1) to provide the title compound (6.6 mg, 18% yield) as a white solid. MS (ESI) m/z=676.5 [M+H]⁺.

### Example 52. 3-(4-((2-((3-([1,4′-Bipiperidin]-1′-yl)phenyl)amino)pyrimidin-4-yl)amino)-1-oxoisoindolin-2-yl)piperidine-2,6-dione (GS-094)

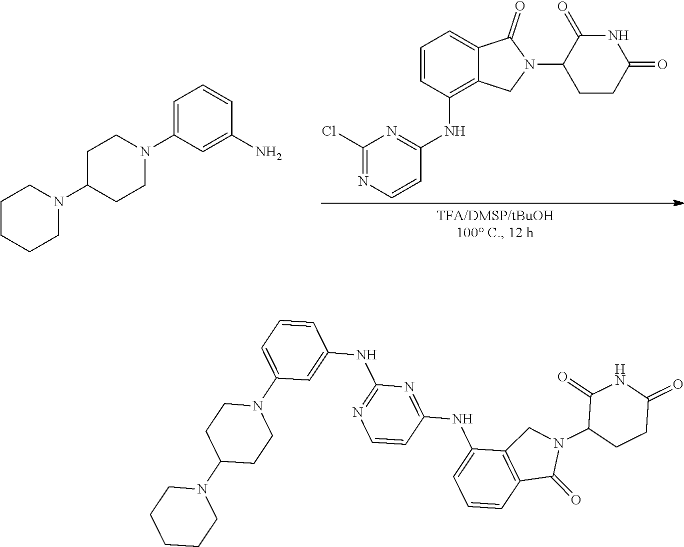 [SMILES: Nc1cccc(N2CCC(N3CCCCC3)CC2)c1 ; O=C1CCC(N2Cc3c(Nc4ccnc(Cl)n4)cccc3C2=O)C(=O)N1 ; O=C1CCC(N2Cc3c(Nc4ccnc(Nc5cccc(N6CCC(N7CCCCC7)CC6)c5)n4)cccc3C2=O)C(=O)N1]

GS-094 was synthesized following the similar procedure for preparing GS-075 (1.1 mg, yield: 2%). MS (ESI) m/z=595.5 [M+H]⁺.

### Example 53. 3-(1-Oxo-6-(6-oxo-6-(4-phenylpiperidin-1-yl)hexyl)isoindolin-2-yl)piperidine-2,6-dione (GS-095)

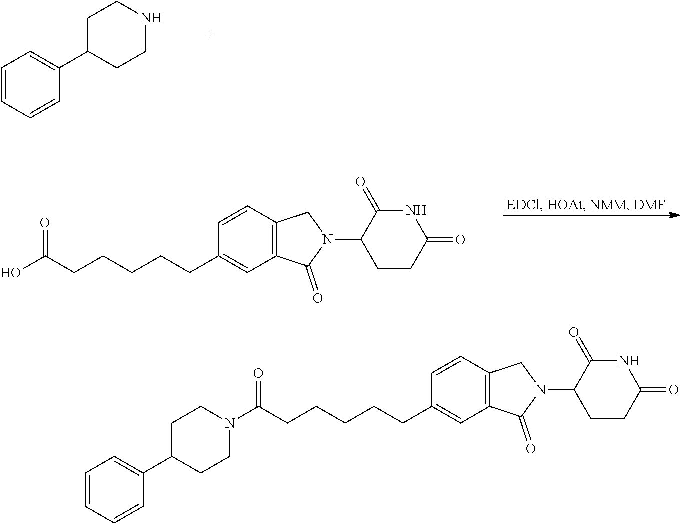 [SMILES: c1ccc(C2CCNCC2)cc1 ; O=C(O)CCCCCc1ccc2c(c1)C(=O)N(C1CCC(=O)NC1=O)C2 ; O=C1CCC(N2Cc3ccc(CCCCCC(=O)N4CCC(c5ccccc5)CC4)cc3C2=O)C(=O)N1]

GS-095 was synthesized following the standard procedure for preparing GS-001 (2.13 mg, yield: 30.4%). MS (ESI) m/z=502.5 [M+H]⁺.

### Example 54. 3-(1-Oxo-6-(7-oxo-7-(4-(pyridin-2-yl)piperazin-1-yl)heptyl)isoindolin-2-yl)piperidine-2,6-dione (GS-096)

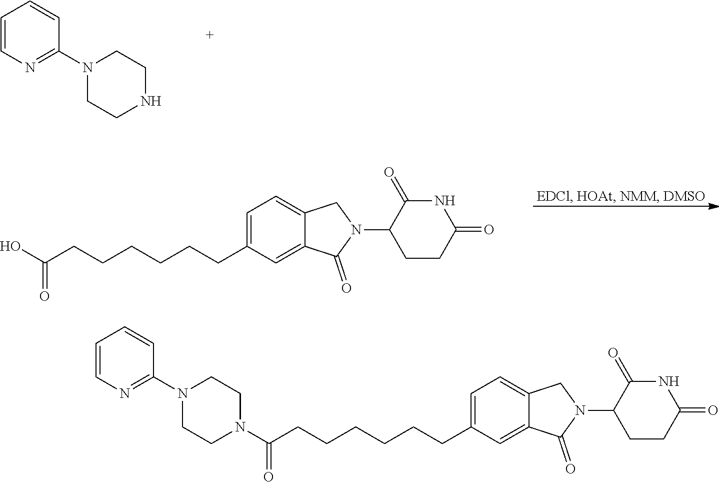 [SMILES: c1ccc(N2CCNCC2)nc1 ; O=C(O)CCCCCCc1ccc2c(c1)C(=O)N(C1CCC(=O)NC1=O)C2 ; O=C1CCC(N2Cc3ccc(CCCCCCC(=O)N4CCN(c5ccccn5)CC4)cc3C2=O)C(=O)N1]

GS-096 was synthesized following the standard procedure or preparing GS-001 (9.6 mg, yield: 69.1%). MS (ESI) m/z=518.6 [M+H]⁺.

### Example 55. 3-(1-Oxo-6-(7-oxo-7-(4-(4-(quinoxalin-2-yl)-1H-pyrazol-1-yl)piperidin-1-yl)heptyl)isoindolin-2-yl)piperidine-2,6-dione (GS-097)

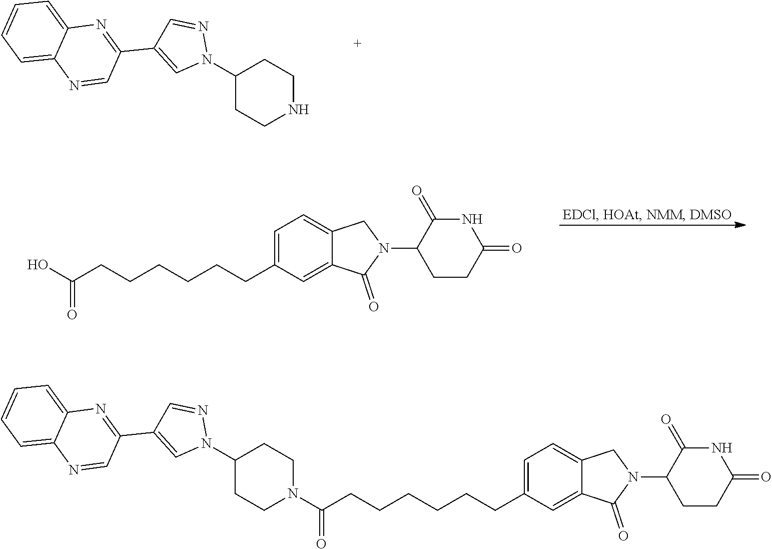 [SMILES: c1ccc2nc(-c3cnn(C4CCNCC4)c3)cnc2c1 ; O=C(O)CCCCCCc1ccc2c(c1)C(=O)N(C1CCC(=O)NC1=O)C2 ; O=C1CCC(N2Cc3ccc(CCCCCCC(=O)N4CCC(n5cc(-c6cnc7ccccc7n6)cn5)CC4)cc3C2=O)C(=O)N1]

GS-097 was synthesized following the standard procedure for preparing GS-001 (10 mg, yield: 58.8%). MS (ESI) m/z=634.7 [M+H]⁺.

### Example 56. 3-(1-Oxo-6-(7-oxo-7-(4-(4-(quinolin-2-yl)-1H-pyrazol-1-yl)piperidin-1-yl)heptyl)isoindolin-2-yl)piperidine-2,6-dione (GS-098)

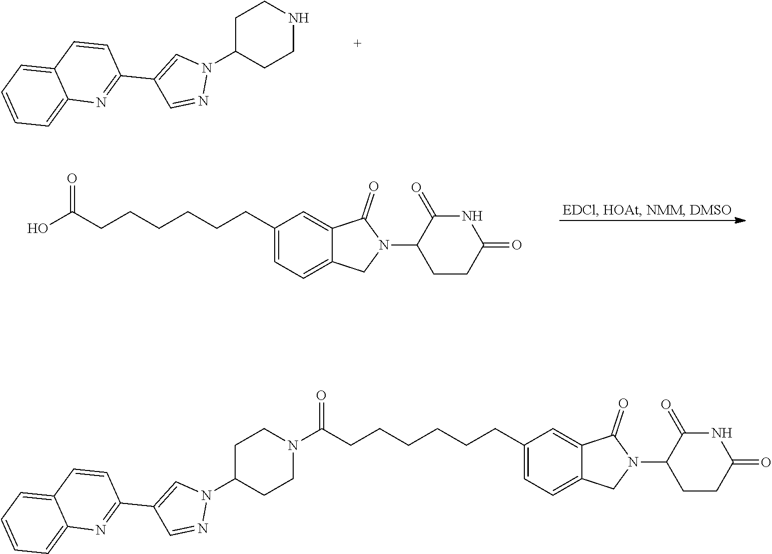 [SMILES: c1ccc2nc(-c3cnn(C4CCNCC4)c3)ccc2c1 ; O=C(O)CCCCCCc1ccc2c(c1)C(=O)N(C1CCC(=O)NC1=O)C2 ; O=C1CCC(N2Cc3ccc(CCCCCCC(=O)N4CCC(n5cc(-c6ccc7ccccc7n6)cn5)CC4)cc3C2=O)C(=O)N1]

GS-098 was synthesized following the standard procedure for preparing GS-001 (5.8 mg, yield: 25.2%) MS (ESI) m/z=633.6 [M+H]⁺.

### Example 57. 3-(1-Oxo-6-(7-oxo-7-(4-phenylpiperidin-1-yl)heptyl)isoindolin-2-yl)piperidine-2,6-dione (GS-099)

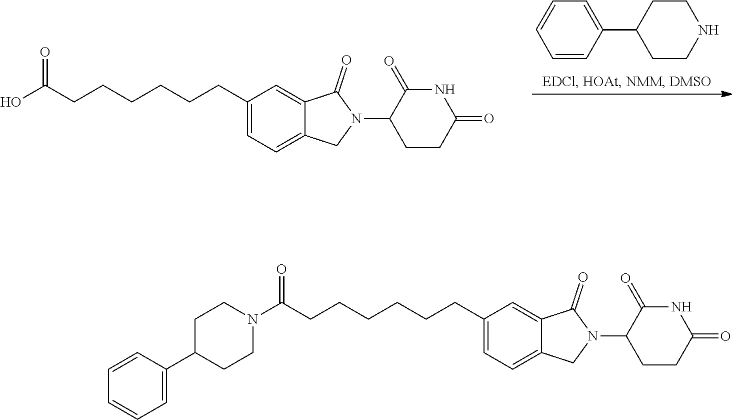 [SMILES: O=C(O)CCCCCCc1ccc2c(c1)C(=O)N(C1CCC(=O)NC1=O)C2 ; c1ccc(C2CCNCC2)cc1 ; O=C1CCC(N2Cc3ccc(CCCCCCC(=O)N4CCC(c5ccccc5)CC4)cc3C2=O)C(=O)N1]

GS-099 was synthesized following the standard procedure for preparing GS-001 (5.6 mg, yield: 37.3%). MS (ESI) m/z=516.6 [M+H]⁺.

### Example 58. 2-(2,6-Dioxopiperidin-3-yl)-5-((2-(4-(4-(quinoxalin-2-yl)-1H-pyrazol-1-yl)cyclohexyl)ethyl)amino)isoindoline-1,3-dione (GS-100)

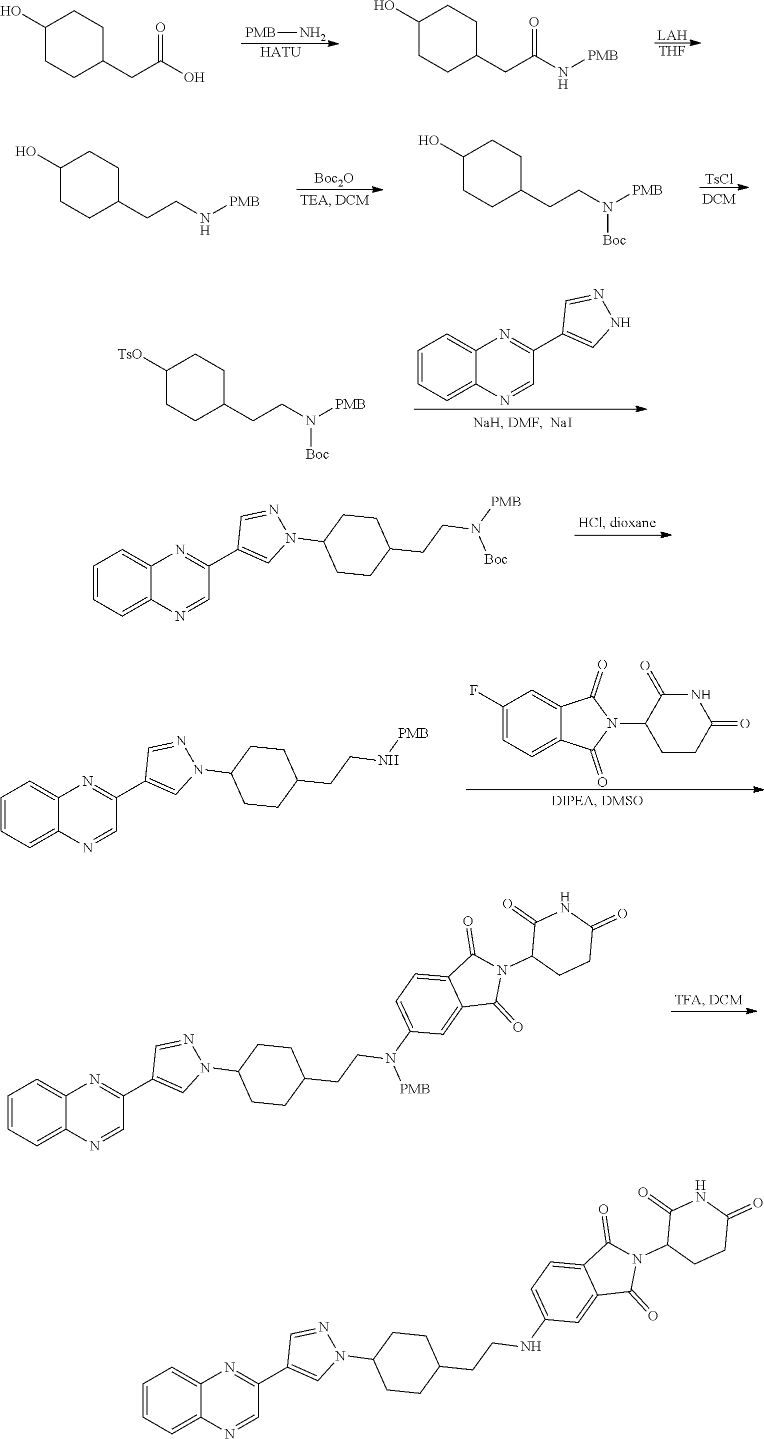

Step 1: Synthesis of 2-(4-hydroxycyclohexyl)-N-(4-methoxybenzyl)acetamide

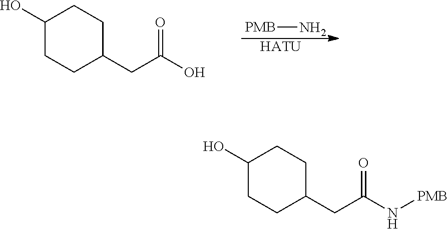

To a solution of 2-(4-hydroxycyclohexyl)acetic acid (800 mg, 5.06 mmol) and (4-methoxyphenyl)methanamine (693.73 mg, 5.06 mmol) in THF (20 mL) were added HOAt (1.37 g, 10.11 mmol), EDCI (1.94 g, 10.11 mmol) and TEA (2.05 g, 20.23 mmol) at rt. The reaction mixture was stirred at rt for 4 h. The reaction was concentrated and purified by silica gel chromatography (petroleum ether/EtOAc=20/1) to provide the title compound (850 mg, 60.6% yield). MS (ESI) m/z=278.3 [M+H]⁺.

Step 2: Synthesis of 4-(2-((4-methoxybenzyl)amino)ethyl)cyclohexan-1-ol

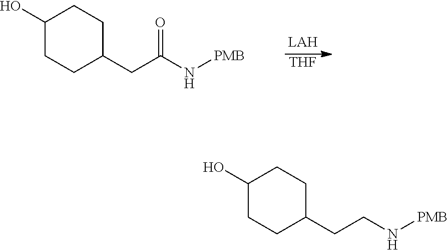

To a solution of 2-(4-hydroxycyclohexyl)-N-[(4-methoxyphenyl)methyl]acetamide (850 mg, 3.06 mmol) in THF (20 mL) was added LAH (415.18 mg, 10.93 mmol) at rt. The mixture was warmed to 68° C. and stirred for 16 h. The reaction was quenched with aq. NaOH (1N, 0.8 mL), filtrated, and concentrated to provide crude title compound (650 mg, 80.7% yield) which was used directly in the next step. MS (ESI) m/z=264.3 [M+H]⁺.

Step 3: Synthesis of tert-butyl (2-(4-hydroxycyclohexyl)ethyl)(4-methoxybenzyl)carbamate

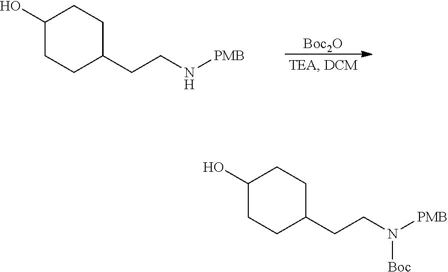

To a solution of 4-[2-[(4-methoxyphenyl)methylamino]ethyl]cyclohexanol (650 mg, 2.47 mmol) in THF (20 mL) were added Boc₂O (810.73 mg, 3.70 mmol) and TEA (998.94 mg, 9.87 mmol) at rt. After the mixture was stirred at rt for 16 h, it was concentrated and purified by silica gel chromatography (DCM/MeOH=20/1) to provide the title compound (820 mg, 91.4% yield). MS (ESI) m/z=308.1 [M−56+H]⁺.

Step 4: Synthesis of 4-(2-((tert-butoxycarbonyl)(4-methoxybenzyl)amino)ethyl)cyclohexyl 4-methylbenzenesulfonate

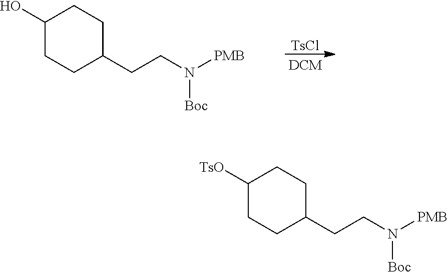

To a solution of tert-butyl (2-(4-hydroxycyclohexyl)ethyl)(4-methoxybenzyl)carbamate (820 mg, 2.26 mmol) in DCM (10 mL) were added 4-methylbenzenesulfonyl chloride (559.11 mg, 2.93 mmol), DMAP (275.60 mg, 2.26 mmol) and TEA (2.28 g, 22.56 mmol) at rt. After the reaction was stirred at rt for 3 h, it was concentrated and purified by silica gel chromatography (petroleum ether/EtOAc=10/1) to provide the title compound (500 mg, 42.8% yield). MS (ESI) m/z=462.3 [M−56+H]⁺.

Step 5: Synthesis of tert-butyl (4-methoxybenzyl)(2-(4-(4-(quinoxalin-2-yl)-1H-pyrazol-1-yl)cyclohexyl)ethyl)carbamate

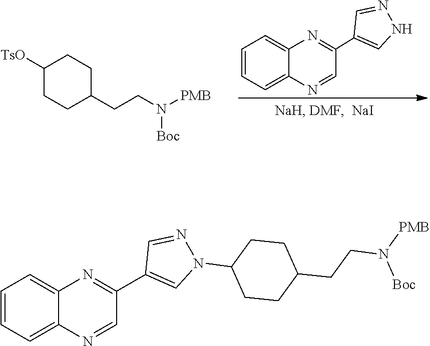

To a solution of 2-(1H-pyrazol-4-yl)quinoxaline (136.45 mg, 695.42 μmol) in DMF (20 mL) was added NaH (55.63 mg, 1.39 mmol, 60% purity) at rt. After the mixture was stirred at rt for 0.5 h, NaI (104.24 mg, 695.42 μmol) and [4-[2-[tert-butoxycarbonyl-[(4-methoxyphenyl)methyl]amino]ethyl] cyclohexyl] 4-methylbenzenesulfonate (360 mg, 695.42 μmol) were added. After the reaction mixture was warmed to 80° C. and stirred for 16 h, it was quenched with aq. NH₄Cl, extracted with DCM, and concentrated. The resulting residue was purified by silica gel chromatography (petroleum ether/EtOAc=3/1 to 1/1) to provide the title compound (130 mg, 34.5% yield). MS (ESI) m/z=542.5 [M+H]⁺.

Step 6: Synthesis of N-(4-methoxybenzyl)-2-(4-(4-(quinoxalin-2-yl)-1H-pyrazol-1-yl)cyclohexyl)ethan-1-amine

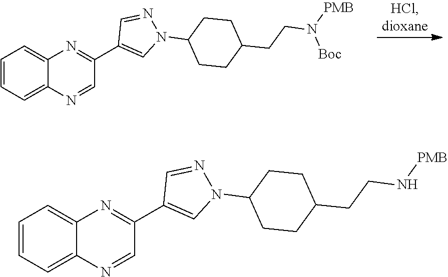

To a solution of tert-butyl N-[4-methoxyphenyl)methyl]-N-[2-[4-(4-quinoxalin-2-ylpyrazol-1-yl)cyclohexyl]ethyl]carbamate (130 mg, 239.99 μmol) in DCM (10 mL) was added HCl/dioxane (1 M, 4 mL) at rt. After the mixture was stirred at rt for 2 h, it was concentrated to provide the title compound (115 mg, 99% yield) which was used directly in the next step. MS (ESI) m/z=442.5 [M+H]⁺.

Step 7: Synthesis of 2-(2,6-dioxopiperidin-3-yl)-5-((4-methoxybenzyl)(2-(4-(4-(quinoxalin-2-yl)-1H-pyrazol-1-yl)cyclohexyl)ethyl)amino)isoindoline-1,3-dione

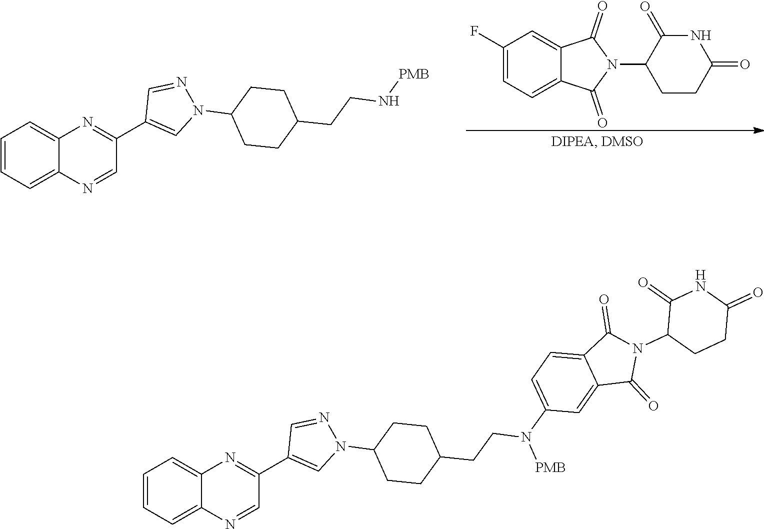

To a solution of N-[(4-methoxyphenyl)methyl]-2-[4-(4-quinoxalin-2-ylpyrazol-1-yl)cyclohexyl]ethanamine (115 mg, 240.57 μmol) and 2-(2,6-dioxo-3-piperidyl)-5-fluoro-isoindoline-1,3-dione (66.45 mg, 240.57 μmol) in DMSO (10 mL) was added DIPEA (186.55 mg, 1.44 mmol) at rt. After the mixture was warmed to 120° C. and stirred for 16 h, it was purified by prep-HPLC to provide crude product which was further purified by prep-TLC (DCM/MeOH=10/1) to provide the title compound (12 mg, 7.2% yield). MS (ESI) m/z=698.6 [M+H]⁺.

Step 8: Synthesis of 2-(2,6-dioxopiperidin-3-yl)-5-((2-(4-(4-(quinoxalin-2-yl)-1H-pyrazol-1-yl)cyclohexyl)ethyl)amino)isoindoline-1,3-dione

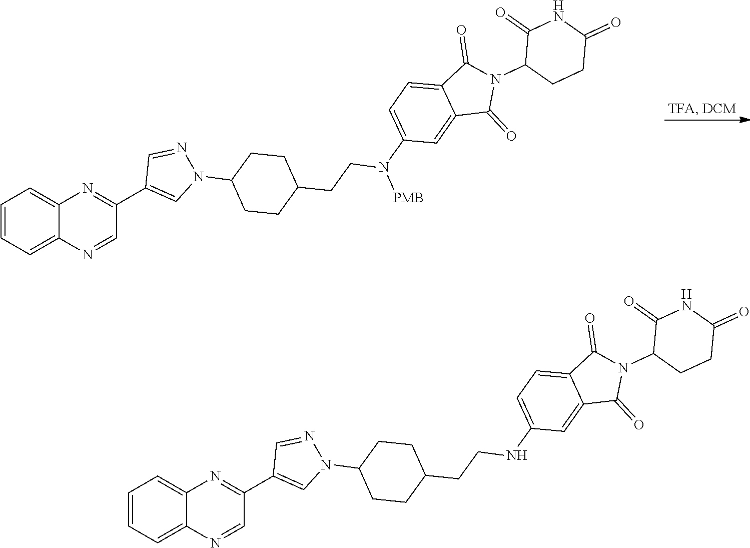

To a solution of 2-(6-dioxo-3-piperidyl)-5-[(4-methoxyphenyl)methyl-[2-[4-(4-quinoxalin-2-ylpyrazol-1-yl)cyclohexyl]ethyl]amino]isoindoline-1,3-dione (12 mg, 17.20 μmol) in DCM (2 mL) was added TFA (2 mL) at rt. After the mixture was stirred at rt for 0.5 h, it was concentrated and purified by prep-TLC (DCM/MeOH=10/1) to provide the title compound (4.2 mg, 42.3% yield) as a yellow solid. MS (ESI) m/z=578.5 [M+H]⁺.

### Example 59. 3-(1-Oxo-6-(6-oxo-6-(4-(4-(quinoxalin-2-yl)-1H-pyrazol-1-yl)piperidin-1-yl)hexyl)isoindolin-2-yl)piperidine-2,6-dione (GS-101)

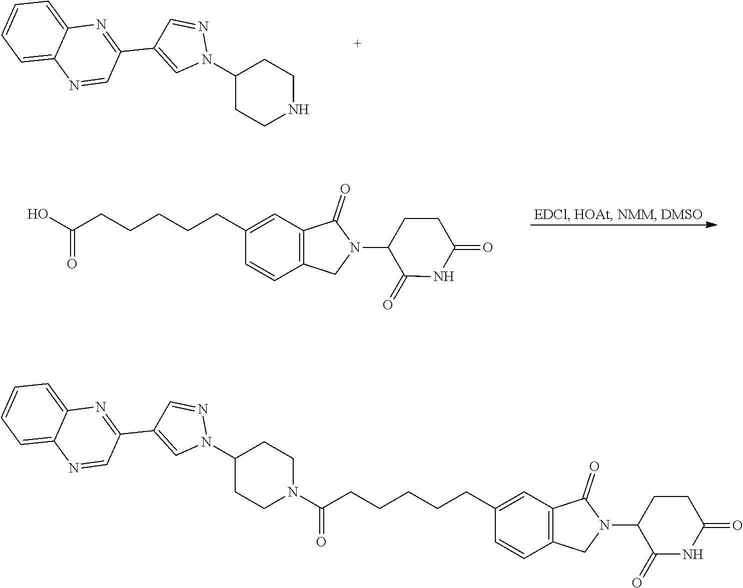 [SMILES: c1ccc2nc(-c3cnn(C4CCNCC4)c3)cnc2c1 ; O=C(O)CCCCCc1ccc2c(c1)C(=O)N(C1CCC(=O)NC1=O)C2 ; O=C1CCC(N2Cc3ccc(CCCCCC(=O)N4CCC(n5cc(-c6cnc7ccccc7n6)cn5)CC4)cc3C2=O)C(=O)N1]

GS-101 was synthesized following the similar procedure for preparing GS-001 (13.3 mg, yield: 77.2%). MS (ESI) m/z=620.7 [M+H]⁺.

### Example 60. 3-(1-Oxo-6-(6-oxo-6-(4-(4-(quinolin-2-yl)-1H-pyrazol-1-yl)piperidin-1-yl)hexyl)isoindolin-2-yl)piperidine-2,6-dione (GS-102)

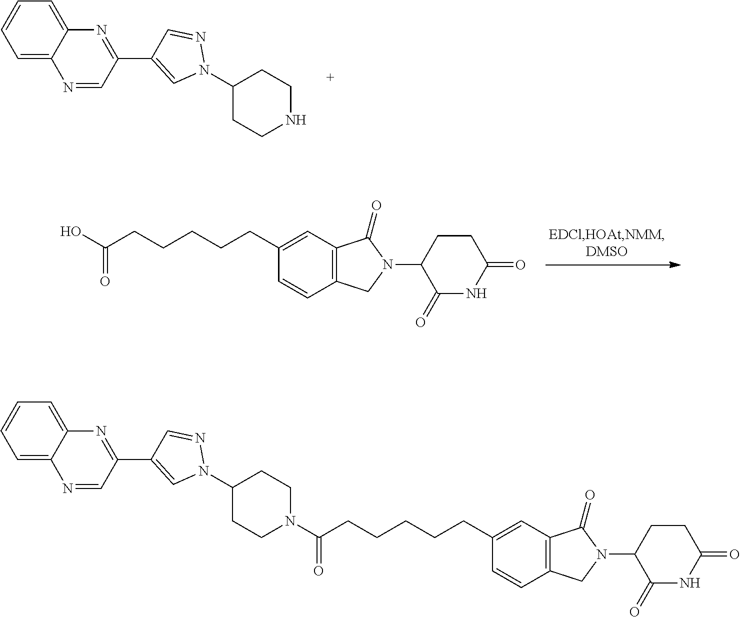 [SMILES: c1ccc2nc(-c3cnn(C4CCNCC4)c3)cnc2c1 ; O=C(O)CCCCCc1ccc2c(c1)C(=O)N(C1CCC(=O)NC1=O)C2 ; O=C1CCC(N2Cc3ccc(CCCCCC(=O)N4CCC(n5cc(-c6cnc7ccccc7n6)cn5)CC4)cc3C2=O)C(=O)N1]

GS-102 was synthesized following the similar procedure for preparing GS-001 (13 mg, yield: 75.1%). MS (ESI) m/z=619.8 [M+H]⁺.

### Example 61. 3-(1-Oxo-6-(6-oxo-6-(4-(pyridin-2-yl)piperazin-1-yl)hexyl)isoindolin-2-yl)piperidine-2,6-dione (GS-103)

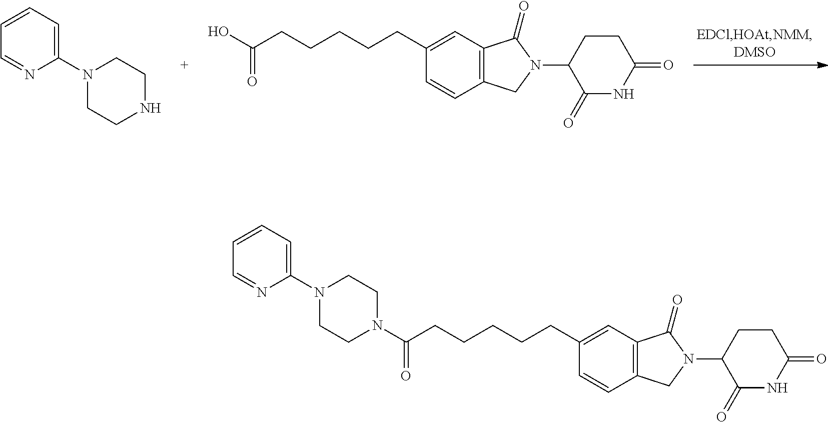 [SMILES: c1ccc(N2CCNCC2)nc1 ; O=C(O)CCCCCc1ccc2c(c1)C(=O)N(C1CCC(=O)NC1=O)C2 ; O=C1CCC(N2Cc3ccc(CCCCCC(=O)N4CCN(c5ccccn5)CC4)cc3C2=O)C(=O)N1]

GS-103 was synthesized following the similar procedure for preparing GS-001 (5.21 mg, yield: 46.4%). MS (ESI) m/z=504.6 [M+H]⁺.

### Example 62. 3-(1-Oxo-6-(6-oxo-6-(4-(pyridin-2-yl)piperidin-1-yl)hexyl)isoindolin-2-yl)piperidine-2,6-dione (GS-104)

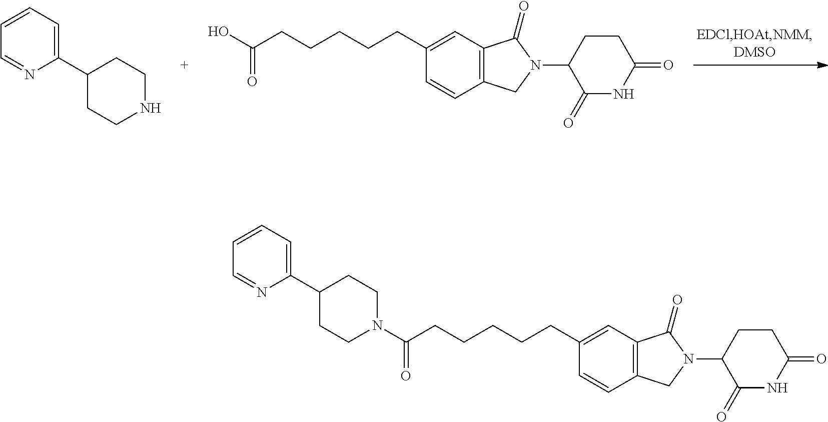 [SMILES: c1ccc(C2CCNCC2)nc1 ; O=C(O)CCCCCc1ccc2c(c1)C(=O)N(C1CCC(=O)NC1=O)C2 ; O=C1CCC(N2Cc3ccc(CCCCCC(=O)N4CCC(c5ccccn5)CC4)cc3C2=O)C(=O)N1]

GS-104 was synthesized following the similar procedure for preparing GS-001 (6.64 mg, yield: 59.2%). MS (ESI) m/z=503.5 [M+H]⁺.

### Example 63. 3-(1-Oxo-6-(7-(4-(quinoxalin-2-yl)-1H-pyrazol-1-yl)heptyl)isoindolin-2-yl)piperidine-2,6-dione (GS-105)

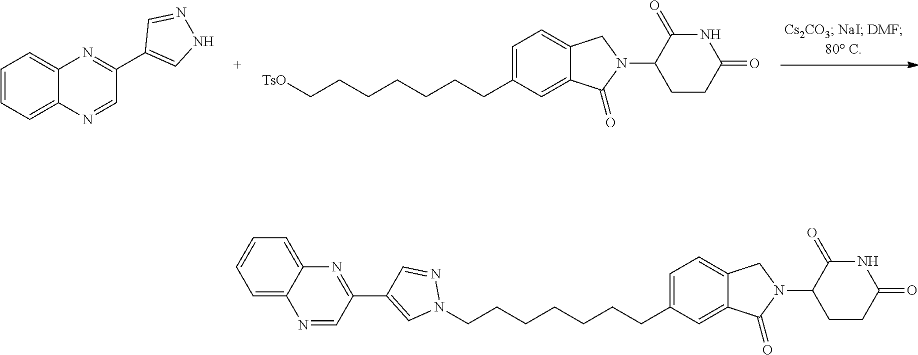 [SMILES: c1ccc2nc(-c3cn[nH]c3)cnc2c1 ; Cc1ccc(S(=O)(=O)OCCCCCCCc2ccc3c(c2)C(=O)N(C2CCC(=O)NC2=O)C3)cc1 ; O=C1CCC(N2Cc3ccc(CCCCCCCn4cc(-c5cnc6ccccc6n5)cn4)cc3C2=O)C(=O)N1]

A mixture of 7-(2-(2,6-dioxopiperidin-3-yl)-3-oxoisoindolin-5-yl)heptyl 4-methylbenzenesulfonate (10.0 mg, 0.02 mmol), 2-(1H-pyrazol-4-yl)quinoxaline (synthesized by a similar procedure for step 1 of CG-563 for synthesis, 5.47 mg, 0.02 mmol), Cs₂CO₃ (12.67 mg, 0.035 mmol) and NaI (2.93 mg, 0.020 mmol) in DMF (0.2 mL) was stirred at 85° C. for 3 h. Then the mixture was purified by C18 column chromatography to provide the title compound (9.8 mg, 91.2% yield) as a white solid. MS (ESI) m/z=537.5 [M+H]⁺.

### Example 64. 3-(1-Oxo-6-(6-(4-(quinoxalin-2-yl)-1H-pyrazol-1-yl)hexyl)isoindolin-2-yl)piperidine-2,6-dione (GS-106)

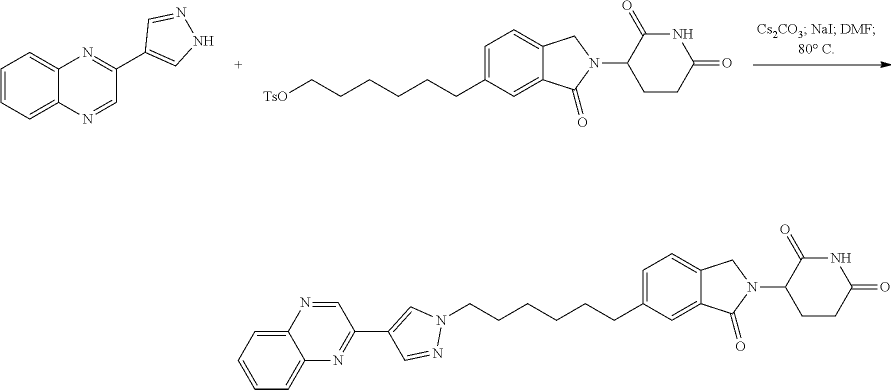 [SMILES: c1ccc2nc(-c3cn[nH]c3)cnc2c1 ; Cc1ccc(S(=O)(=O)OCCCCCCc2ccc3c(c2)C(=O)N(C2CCC(=O)NC2=O)C3)cc1 ; O=C1CCC(N2Cc3ccc(CCCCCCn4cc(-c5cnc6ccccc6n5)cn4)cc3C2=O)C(=O)N1]

GS-106 was synthesized following the similar procedure for preparing GS-105 (8.2 mg, yield: 78.4%). MS (ESI) m/z=523.5 [M+H]⁺.

### Example 65. 2-(2,6-Dioxopiperidin-3-yl)-5-((3-(3-(4-(quinoxalin-2-yl)-1H-pyrazol-1-yl)cyclobutyl)propyl)amino)isoindoline-1,3-dione (GS-107)

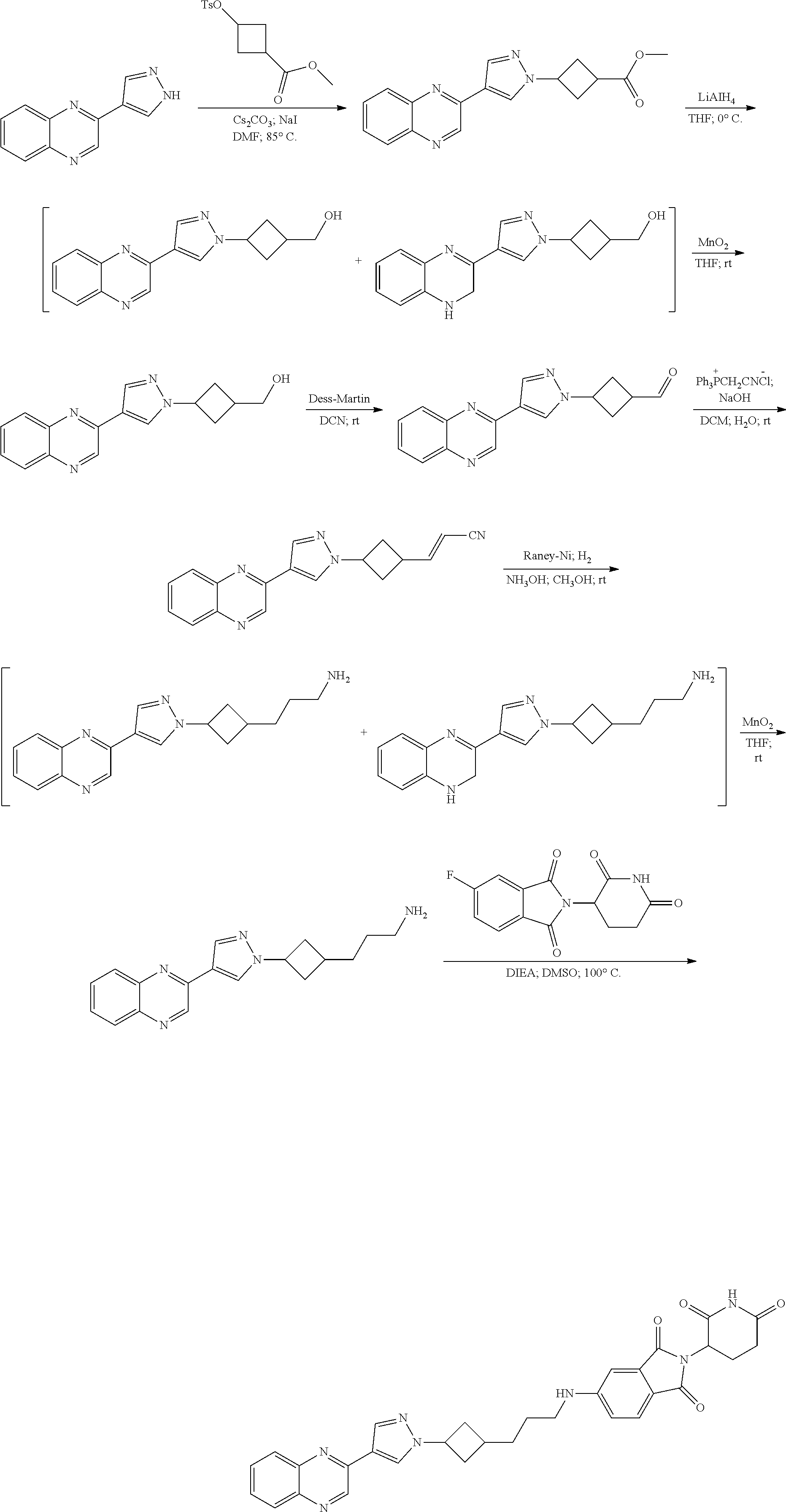

Step 1: Synthesis of methyl 3-(4-(quinoxalin-2-yl)-1H-pyrazol-1-yl)cyclobutane-1-carboxylate

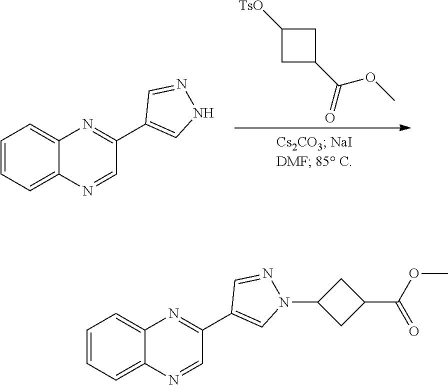 [SMILES: c1ccc2nc(-c3cn[nH]c3)cnc2c1 ; COC(=O)C1CC(OS(=O)(=O)c2ccc(C)cc2)C1 ; COC(=O)C1CC(n2cc(-c3cnc4ccccc4n3)cn2)C1]

A mixture of methyl 3-(tosyloxy)cyclobutane-1-carboxylate (28.98 mg, 0.102 mmol), 2-(1H-pyrazol-4-yl)quinoxaline (10.0 mg, 0.051 mmol), Cs₂CO₃ (49.73 mg, 0.153 mmol) and NaI (11.48 mg, 0.077 mmol) in DMF (1 mL) was stirred at 85° C. for 3 h. Then the mixture was purified by C18 column chromatography to provide the title compound (8 mg, 51% yield) as a white solid. MS (ESI) m/z=309.3 [M+H]⁺.

Step 2: Synthesis of (3-(4-(quinoxalin-2-yl)-1H-pyrazol-1-yl)cyclobutyl)methanol

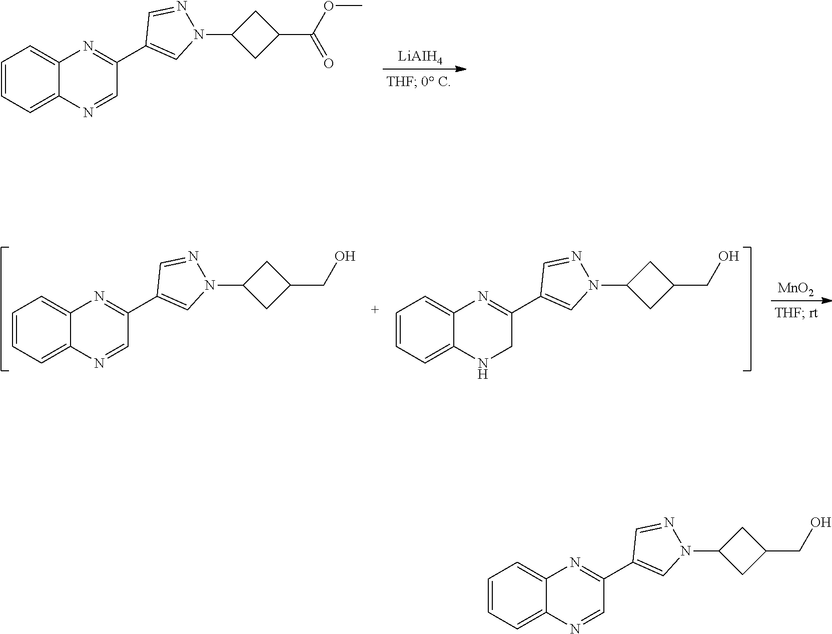

To a mixture of LiAlH₄ (9.48 mg, 0.25 mmol) in THF (1 mL) was added methyl 3-(4-(quinoxalin-2-yl)-1H-pyrazol-1-yl)cyclobutane-1-carboxylate (10 mg, 0.025 mmol) in THF (1 mL) dropwise at 0° C. After the mixture was stirred at 0° C. for 1 h, H₂O (0.01 mL), 15% aq.NaOH (0.01 mL) and H₂O (0.01 mL) were added into the mixture. The precipitate was filtered off. The filtrate was concentrated. The resulting residue was dissolved in THF (2 mL). MnO₂ (10 mg) was added, and the mixture was stirred at rt for 10 min. The reaction mixture was filtered, and the filtrate was concentrated to provide the title compound (10 mg, yield: 99.9%) as white solid. MS (ESI) m/z=281.1 [M+H]⁺.

Step 3: Synthesis of 3-(4-(quinoxalin-2-yl)-1H-pyrazol-1-yl)cyclobutane-1-carbaldehyde

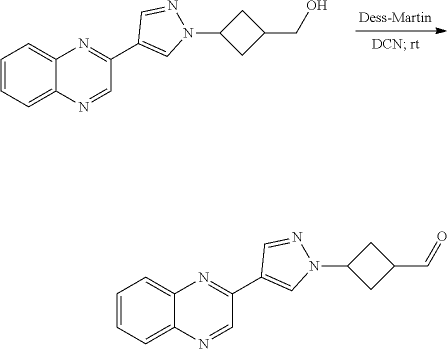 [SMILES: OCC1CC(n2cc(-c3cnc4ccccc4n3)cn2)C1 ; O=CC1CC(n2cc(-c3cnc4ccccc4n3)cn2)C1]

To a solution of (3-(4-(quinoxalin-2-yl)-1H-pyrazol-1-yl)cyclobutyl)methanol (10 mg, 0.036 mmol) in anhydrous dichloromethane (1 mL) at 0° C. was added 1,1,1-tris(acetyloxy)-1,1-dihydro-1,2-benzodioxol-3-(1H)-one (30.54 mg, 0.072 mmol). The reaction was stirred at rt under nitrogen for 3 h, before it was diluted with DCM, washed with brine, dried over Na₂SO₄, filtered, and concentrated. The resulting residue was purified by prep-TLC to provide the title compound (8 mg, yield: 79.9%). MS (ESI) m/z=279.1 [M+H]⁺.

Step 4: Synthesis of (E)-3-(3-(4-(quinoxalin-2-yl)-1H-pyrazol-1-yl)cyclobutyl)acrylonitrile

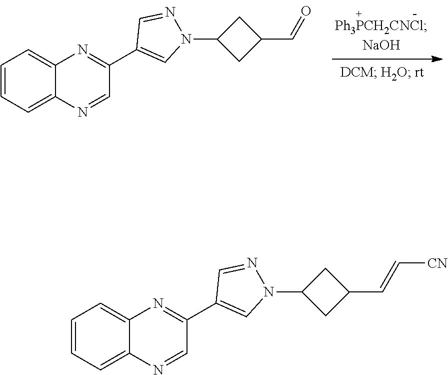 [SMILES: O=CC1CC(n2cc(-c3cnc4ccccc4n3)cn2)C1 ; N#C/C=C/C1CC(n2cc(-c3cnc4ccccc4n3)cn2)C1]

To a solution of (cyanomethyl)triphenylphosphonium chloride (12.15 mg, 0.036 mmol) and NaOH (12 mg, 0.3 mmol) in water (0.1 mL) and DCM (0.3 mL) was added 3-(4-(quinoxalin-2-yl)-1H-pyrazol-1-yl)cyclobutane-1-carbaldehyde (10 mg, 0.036 mmol) at 0° C. The mixture was stirred at rt for 4 h. The mixture was diluted with DCM (5 mL) and water (5 mL). The organic phase was washed with water (5 mL), brine (5 mL), dried over anhydrous sodium sulfate, and concentrated. The resulting residue was purified by flash chromatography to provide the title compound (6 mg, 55.5% yield) as a white solid. MS (ESI) m/z=302.1 [M+H]⁺.

Step 5: Synthesis of 3-(3-(4-(quinoxalin-2-yl)-1H-pyrazol-1-yl)cyclobutyl)propan-1-amine

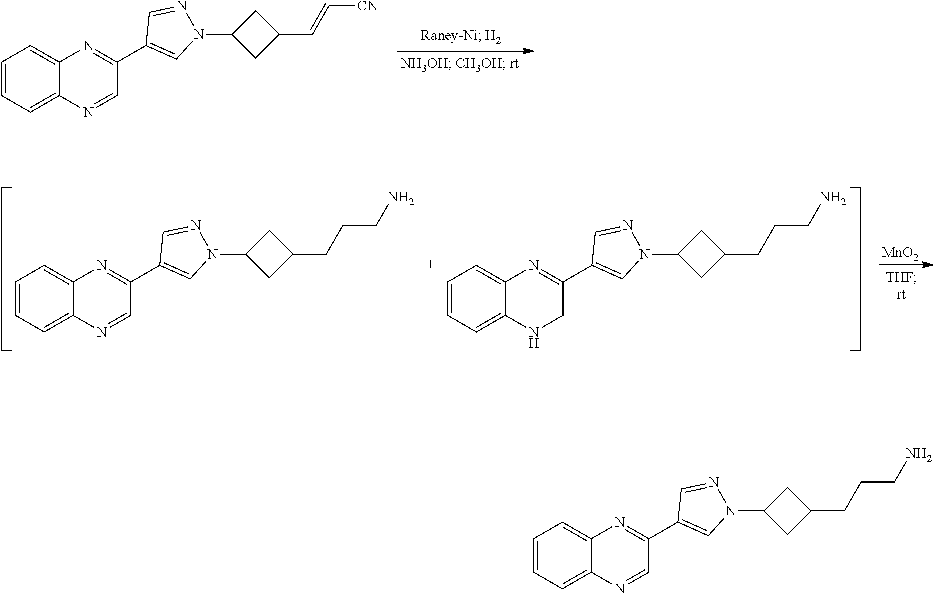

To a solution of (E)-3-(3-(4-(quinoxalin-2-yl)-1H-pyrazol-1-yl)cyclobutyl)acrylonitrile (6 mg, 0.02 mmol) in MeOH (0.5 mL) was added ammonia (0.1 mL) and Raney Ni (6 mg). The reaction mixture was purged with hydrogen and shaken under an atmosphere of hydrogen (45 psi) at rt for 6 h using a standard hydrogenation apparatus. The reaction mixture was filtered, and the filtrate was concentrated. The resulting residue was dissolved in THF (2 mL), before MnO₂ (6 mg) was added. After the mixture was stirred at rt for 10 min, it was filtered. The filtrate was concentrated to provide the title compound (6 mg, yield: 99.9%) as white solid. MS (ESI) m/z=308.2 [M+H]⁺.

Step 6: Synthesis of 2-(2,6-dioxopiperidin-3-yl)-5-((3-(3-(4-(quinoxalin-2-yl)-1H-pyrazol-1-yl)cyclobutyl)propyl)amino)isoindoline-1,3-dione

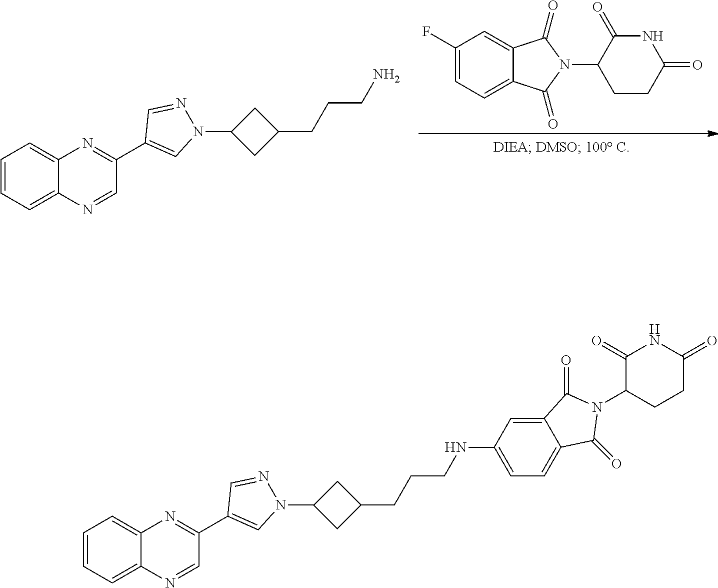 [SMILES: NCCCC1CC(n2cc(-c3cnc4ccccc4n3)cn2)C1 ; O=C1CCC(N2C(=O)c3ccc(F)cc3C2=O)C(=O)N1 ; O=C1CCC(N2C(=O)c3ccc(NCCCC4CC(n5cc(-c6cnc7ccccc7n6)cn5)C4)cc3C2=O)C(=O)N1]

To a solution of 3-(3-(4-(quinoxalin-2-yl)-1H-pyrazol-1-yl)cyclobutyl)propan-1-amine (6 mg, 0.02 mmol) in DMSO (0.5 mL) were added 2-(2,6-dioxopiperidin-3-yl)-5-fluoroisoindoline-1,3-dione (5.52 mg, 0.02 mmol) and DIEA (0.1 mL). After it was stirred at 100° C. for 5 h, the mixture was purified by prep-HPLC to provide the title compound (1.2 mg, yield: 10.7%) as a yellow solid. (ESI) m/z=564.4 [M+H]⁺.

### Example 66. 3-(1-Oxo-4-((4-((4-(4-(quinoxalin-2-yl)-1H-pyrazol-1-yl)piperidin-1-yl)methyl)benzyl)oxy)isoindolin-2-yl)piperidine-2,6-dione (GS-108)

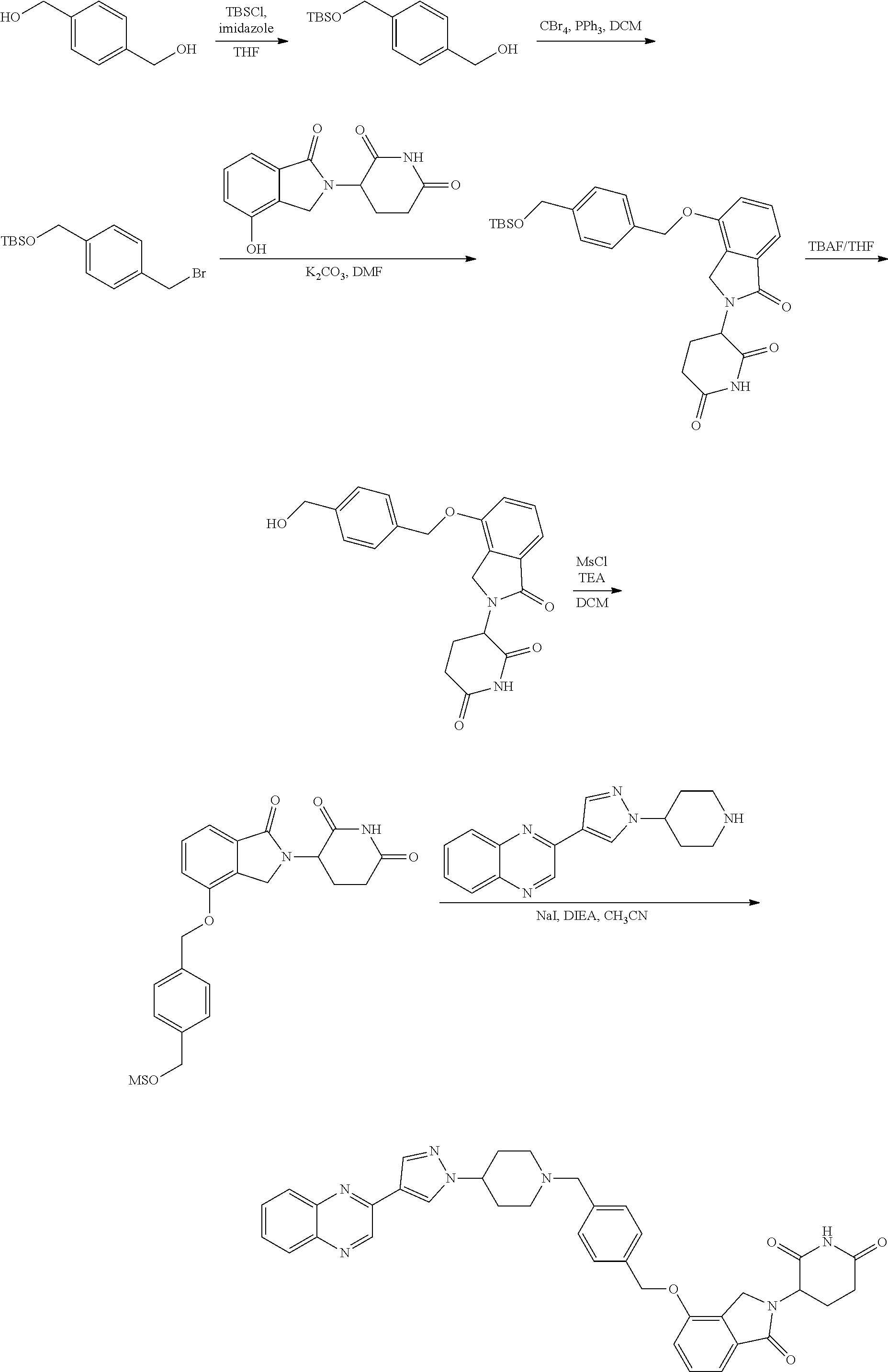

Step 1: Synthesis of (4-(((tert-butyldimethylsilyl)oxy)methyl)phenyl)methanol

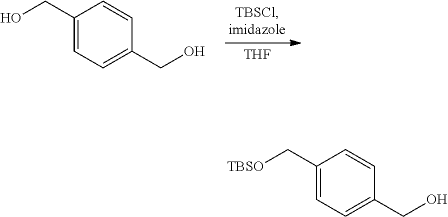 [SMILES: OCc1ccc(CO)cc1 ; CC(C)(C)[Si](C)(C)OCc1ccc(CO)cc1 ; "THF"]

To a solution of 1,4-phenylenedimethanol (13.8 g, 99.88 mmol) in DCM (400 mL) was added imidazole (13.6 g, 199.76 mmol) followed by TBSCl (15.05 g, 99.88 mmol) at 0° C. The resulting mixture was stirred at rt for 16 h, before it was concentrated and The resulting residue was purified by silica gel chromatography (petroleum ether/EtOAc=10:1 to 5:1) to provide the desired product (15 g, yield: 59%) as a colorless oil.

Step 2: Synthesis of ((4-(bromomethyl)benzyl)oxy)(tert-butyl)dimethylsilane

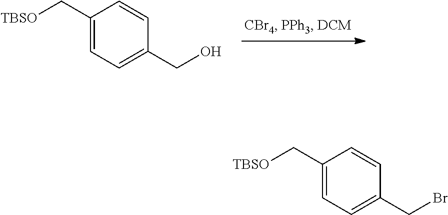 [SMILES: CC(C)(C)[Si](C)(C)OCc1ccc(CO)cc1 ; CC(C)(C)[Si](C)(C)OCc1ccc(CBr)cc1]

To a solution of ((4-(((tert-butyldimethylsiyl)oxymethyl)phenyl)methanol (252 mg, 1 mmol) in DCM (10 mL) were added CBr₄ (496 mg, 1.5 mmol) and PPh₃ (392 mg, 1.5 mmol) at 0° C. The resulting mixture was stirred at rt for 4 h, before it was concentrated and The resulting residue was purified by prep-TLC to provide desired product (240 mg, yield: 76%) as a white solid.

Step 3: Synthesis of 3-(4-((4-(hydroxymethyl)benzyl)oxy)-1-oxoisoindolin-2-yl)piperidine-2,6-dione

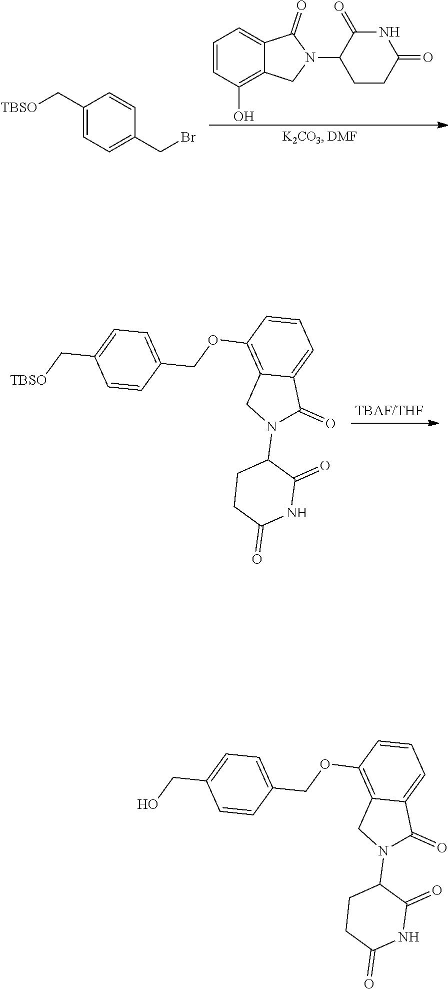 [SMILES: CC(C)(C)[Si](C)(C)OCc1ccc(CBr)cc1 ; O=C1CCC(N2Cc3c(O)cccc3C2=O)C(=O)N1 ; CC(C)(C)[Si](C)(C)OCc1ccc(COc2cccc3c2CN(C2CCC(=O)NC2=O)C3=O)cc1 ; O=C1CCC(N2Cc3c(OCc4ccc(CO)cc4)cccc3C2=O)C(=O)N1]

To a solution of ((4-(bromomethyl)benzyl)oxy)(tert-butyl)dimethylsilane (770 mg, 2.44 mmol) and 3-(4-hydroxy-1-oxoisoindolin-2-yl)piperidine-2,6-dione (635 mg, 2.44 mmol) in DMF (10 mL) was added K₂CO₃ (674 mg, 4.88 mmol). The resulting mixture was stirred at 40° C. for 16 h, before TBAF (2.55 g, 9.77 mmol) was added. The reaction mixture was purified by prep-HPLC to provide desired product (450 mg, yield: 48%) as a white solid. MS (ESI) m/z=381.3 [M+H]⁺.

Step 4: Synthesis of 4-(((2-(2,6-dioxopiperidin-3-yl)-1-oxoisoindolin-4-yl)oxy)methyl)benzyl methanesulfonate

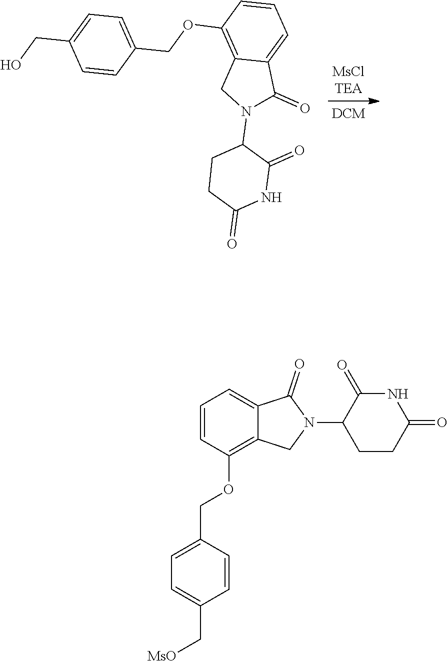 [SMILES: O=C1CCC(N2Cc3c(OCc4ccc(CO)cc4)cccc3C2=O)C(=O)N1 ; CS(=O)(=O)OCc1ccc(COc2cccc3c2CN(C2CCC(=O)NC2=O)C3=O)cc1]

To a solution of 3-(4-((4-(hydroxymethyl)benzyl)oxy)-1-oxoisoindolin-2-yl)piperidine-2,6-dione (25 mg, 0.066 mmol) and TEA (13 mg, 0.132 mmol) in DCM (2 mL) was added MsCl (15 mg, 0.132 mmol). The reaction mixture was stirred at rt for 2 h, before it was concentrated to provide the crude product which was used directly in the next step without further purification.

Step 5: 3-(1-oxo-4-((4-((4-(4-(quinoxalin-2-yl)-1H-pyrazol-1-yl)piperidin-1-yl)methyl)benzyl)oxy)isoindolin-2-yl)piperidine-2,6-dione

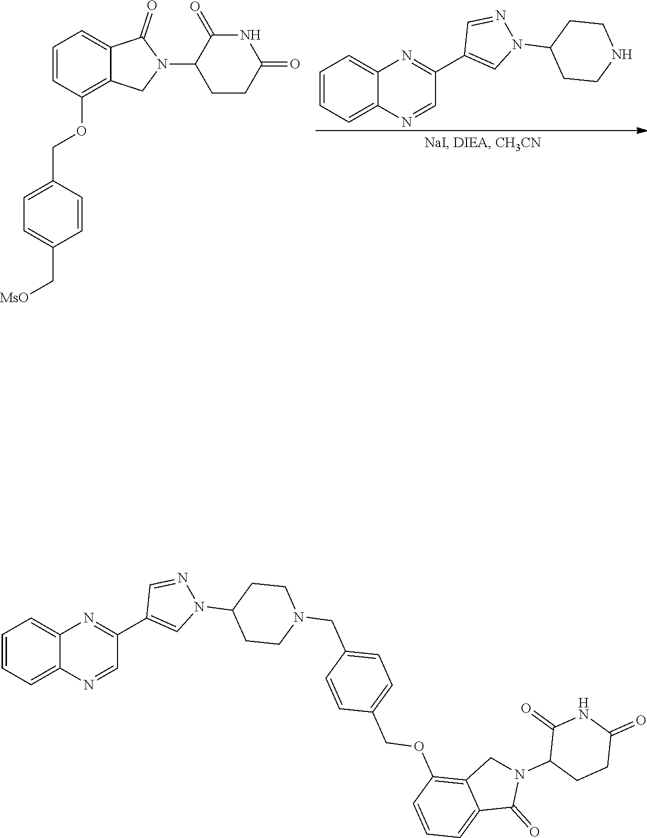

The mixture of [4-[[2-(2,6-dioxo-3-piperidyl)-1-oxo-isoindolin-4-yl]oxymethyl]phenyl]methyl methanesulfonate (30 mg, 0.064 mmol), DIEA (25 mg, 0.19 mmol), NaI (19 mg, 0.13 mmol) and 2-[1-(4-piperidyl)pyrazol-4-yl]quinoxaline (18 mg, 0.064 mmol) in CH₃CN (3 mL) was stirred at 70° C. for 12 h. The mixture was purified by reserve phase column chromatography to provide the title compound (19 mg, 46% yield) as a yellow solid. MS (ESI) m/z=642.6 [M+H]⁻.

### Example 67. N-((2-(2,6-Dioxopiperidin-3-yl)-1-oxoisoindolin-5-yl)methyl)-2-(2-(4-(quinoxalin-2-yl)-1H-pyrazol-1-yl)cyclopropyl)acetamide (GS-109)

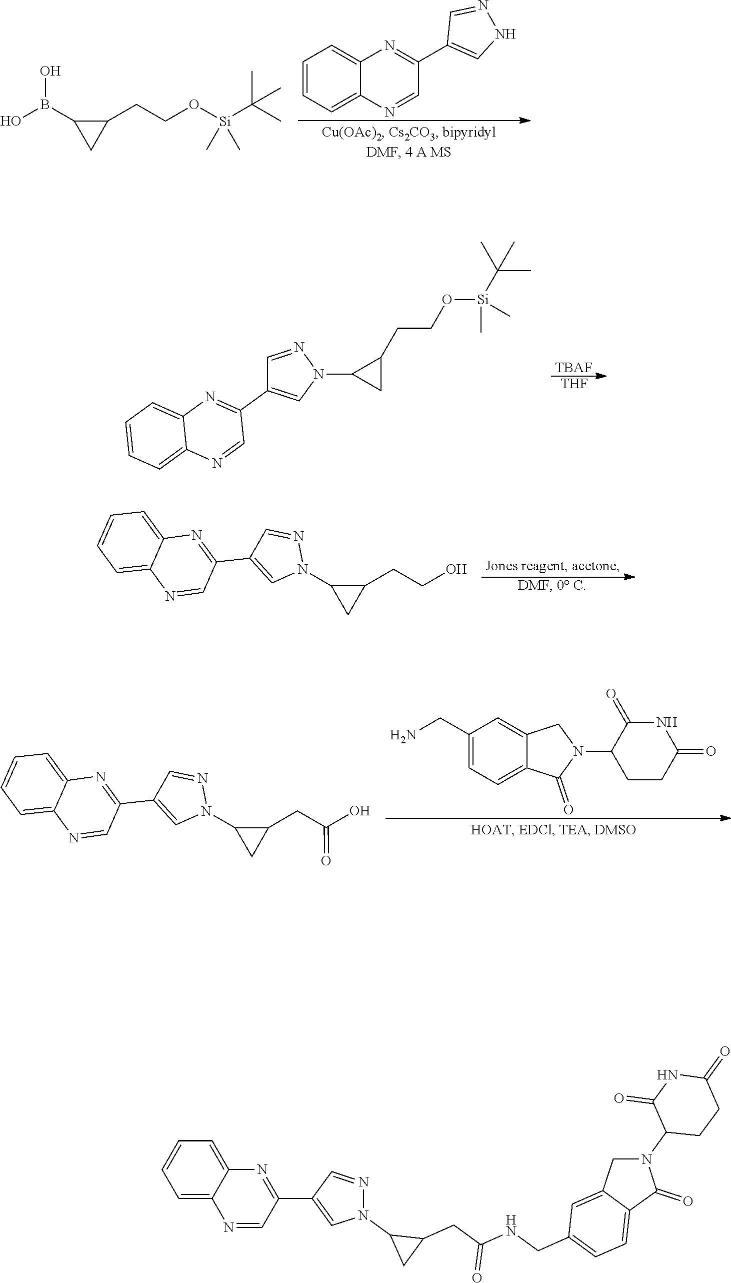

Step 1: Synthesis of 2-(1-(2-(2-((tert-butyldimethylsilyl)oxy)ethyl)cyclopropyl)-1H-pyrazol-4-yl)quinoxaline

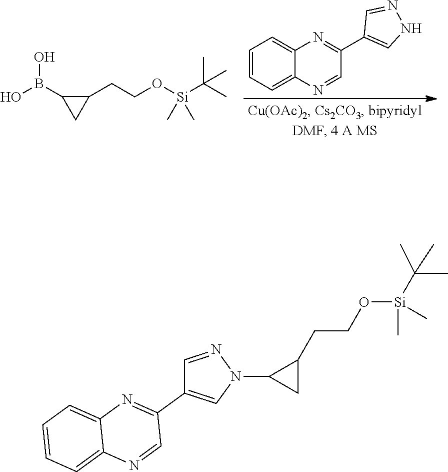

To a solution of 2-(1-pyrazol-4-yl)quinoxaline (200 mg, 1.02 mmol) and [2-[2-[tert-butyl(dimethyl)silyl]oxyethyl]cyclopropyl]boronic acid (497.86 mg, 2.04 mmol) in DMF (15 mL) were added Cu(OAc)₂ (369.00 mg, 2.04 mmol), Cs₂CO₃ (662.56 mg, 2.04 mmol), bipyridyl (318.40 mg, 2.04 mmol) and 4 Å molecular sieves (100 mg). The reaction mixture was warmed to 100° C. and stirred for 16 h. The reaction was purified by prep-HPLC to provide the title compound (150 mg, 37.3% yield). MS (ESI) m/z=395.5 [M+H]⁺.

Step 2: Synthesis of N-((2-(2,6-dioxopiperidin-3-yl)-1-oxoisoindolin-5-yl)methyl)-2-(2-(4-(quinoxalin-2-yl)-1H-pyrazol-1-yl)cyclopropyl)acetamide

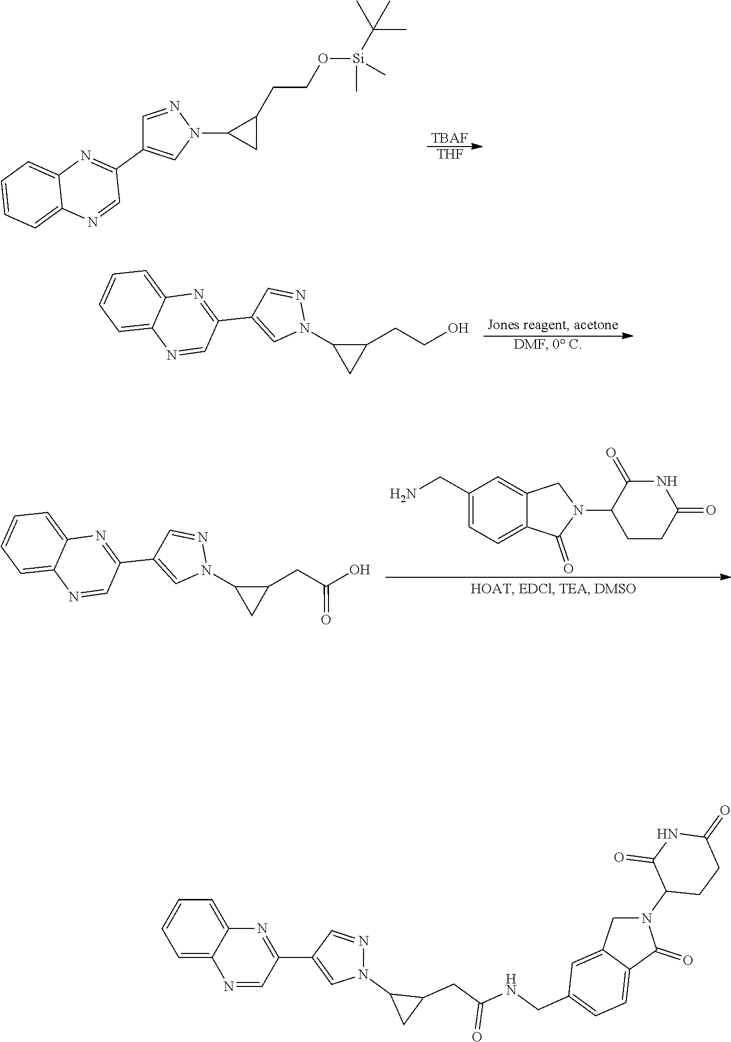

GS-109 was synthesized following the similar procedure for preparing GS-083 (15 mg, yield: 19.1%). MS (ESI) m/z=550.5 [M+H]⁺.

### Example 68. 3-(1-Oxo-4-((3-((3-(4-(4-(quinoxalin-2-yl)-1H-pyrazol-1-yl)piperidin-1-yl)phenyl)amino)cyclohexyl)amino)isoindolin-2-yl)piperidine-2,6-dione (GS-110)

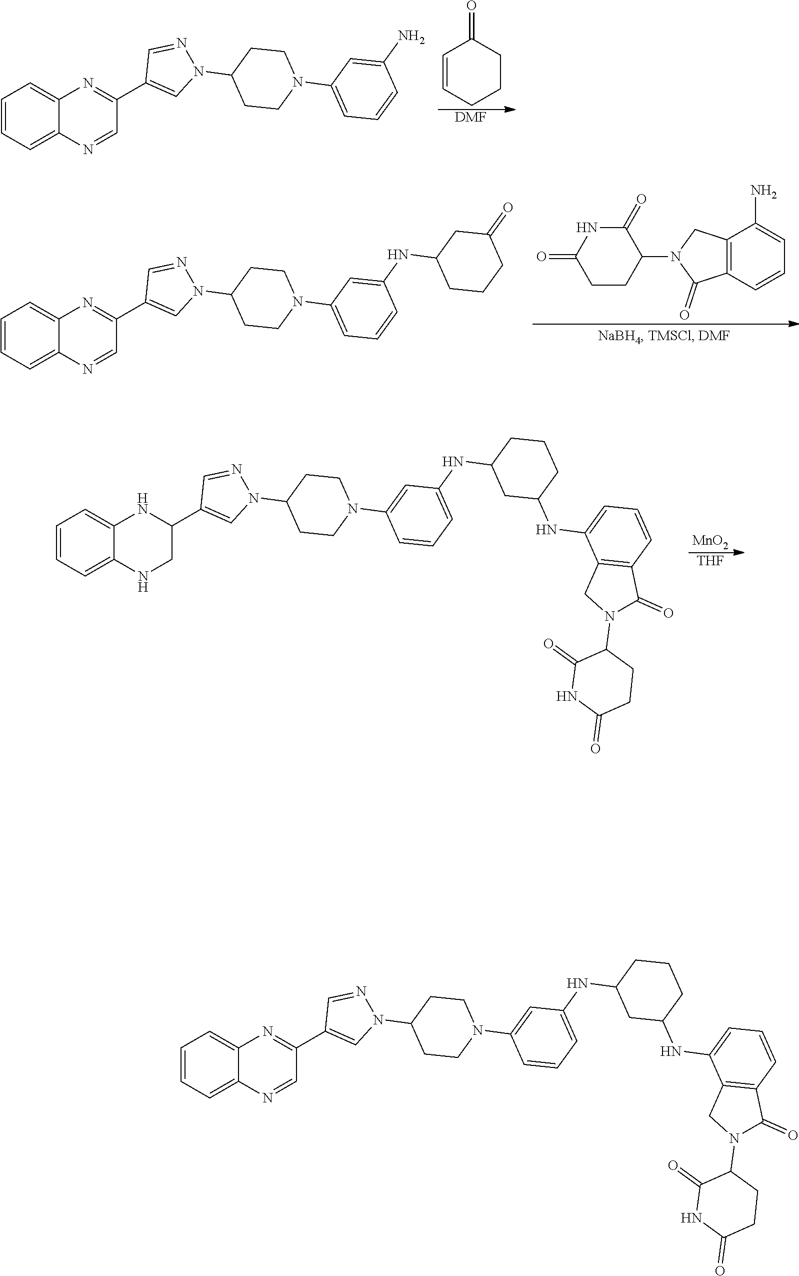

Step 1: Synthesis of 3-((3-(4-(4-(quinoxalin-2-yl)-1H-pyrazol-1-yl)piperidin-1-yl)phenyl)amino)cyclohexan-1-one

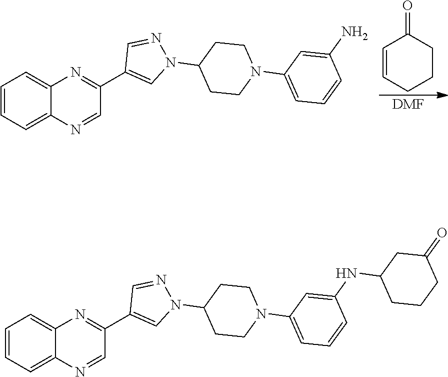

A mixture of 3-[4-(4-quinoxalin-2-ylpyrazol-1-yl)-1-piperidyl]aniline (350 mg, 0.94 mmol) and cyclohex-2-en-1-one (118 mg, 1.23 mmol) in THF (0.5 mL) was stirred at rt for 18 min. The resulting mixture was purified by prep-HPLC to provide the title compound (135 mg, 30.6% yield) as a yellow solid. MS (ESI) m/z=467.5 [M+H]⁺.

Step 2: Synthesis of 3-(1-oxo-4-((3-((3-(4-(4-(1,2,3,4-tetrahydroquinoxalin-2-yl)-1H-pyrazol-1-yl)piperidin-1-yl)phenyl)amino)cyclohexyl)amino)isoindolin-2-yl)piperidine-2,6-dione

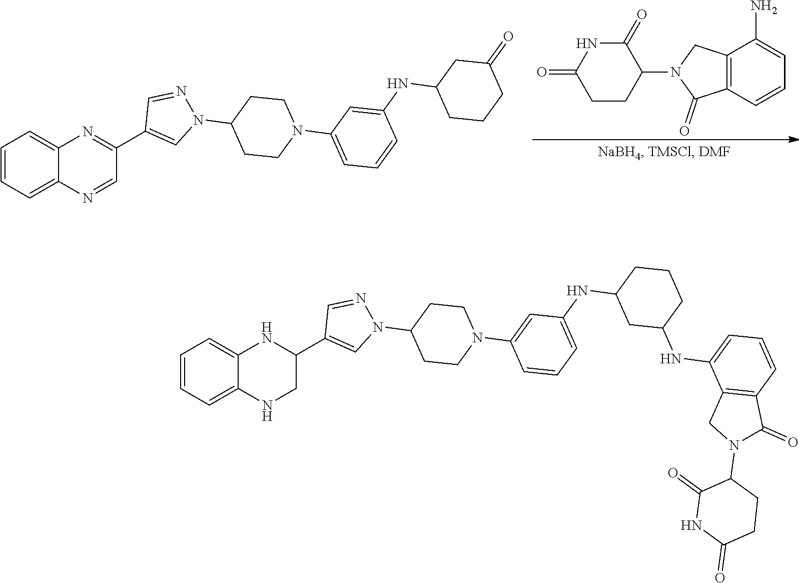

A mixture of 3-[3-[4-(4-quinoxalin-2-ylpyrazol-1-yl)-1-piperidyl]anilino]cyclohexanone (103 mg, 0.22 mmol) and 3-(4-amino-1-oxo-isoindolin-2-yl)piperidine-2,6-dione (86 mg, 0.33 mmol) in DMF (4 mL) was stirred at 0° C. for 2 min, before TMSCI (72 mg, 0.66 mmol) was added. The reaction was stirred at 0° C. for 40 min, at which time, NaBH₄ (12.5 mg, 0.33 mmol) was added and the reaction was stirred for another 12 h. The mixture was purified by reverse-phase chromatography to provide the title compound (40 mg, 25.4% yield). MS (ESI) m/z=714.6 [M+H]⁺.

Step 3: Synthesis of 3-(1-oxo-4-((3-((3-(4-(4-(quinoxalin-2-yl)-1H-pyrazol-1-yl)piperidin-1-yl)phenyl)amino)cyclohexyl)amino)isoindolin-2-yl)piperidine-2,6-dione

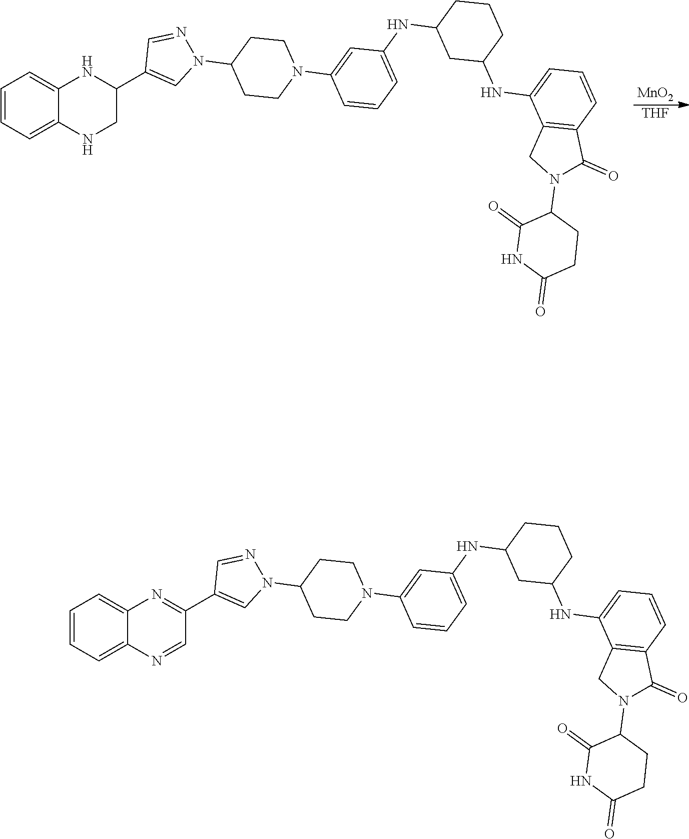

A mixture of 3-[1-oxo-4-[[3-[3-[4-[4-(1,2,3,4-tetrahydroquinoxalin-2-yl)pyrazol-1-yl]-1-piperidyl]anilino]cyclohexyl]amino]isoindolin-2-yl]piperidine-2,6-dione (40 mg, 0.056 mmol) and MnO₂ (48.75 mg, 0.56 mmol) in THF (5 mL) was stirred at 25° C. for 10 min. MnO₂ was removed by filtration, the filtrate was purified by reverse-phase chromatography, followed by prep-TLC (MeOH/DCM=1:13) to provide the title compound (4.7 mg, 11.8% yield) as a light-yellow solid. MS (ESI) m/z=710.7 [M+H]⁺.

### Example 69. 3-(1-Oxo-6-(7-oxo-7-(4-(pyridin-2-yl)piperidin-1-yl)heptyl)isoindolin-2-yl)piperidine-2,6-dione (GS-111)

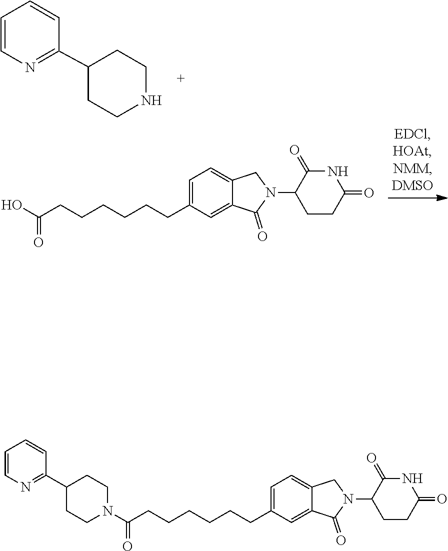 [SMILES: c1ccc(C2CCNCC2)nc1 ; O=C(O)CCCCCCc1ccc2c(c1)C(=O)N(C1CCC(=O)NC1=O)C2 ; O=C1CCC(N2Cc3ccc(CCCCCCC(=O)N4CCC(c5ccccn5)CC4)cc3C2=O)C(=O)N1]

GS-111 was synthesized following the similar procedure for preparing GS-001 (9.6 mg, yield: 30.1%). MS (ESI) m/z=517.7 [M+H]⁺.

### Example 70. 2-(2,6-Dioxopiperidin-3-yl)-5-((3-(3-(4-(quinoxalin-2-yl)-1H-pyrazol-1-yl)cyclohexyl)propyl)amino)isoindoline-1,3-dione (GS-112)

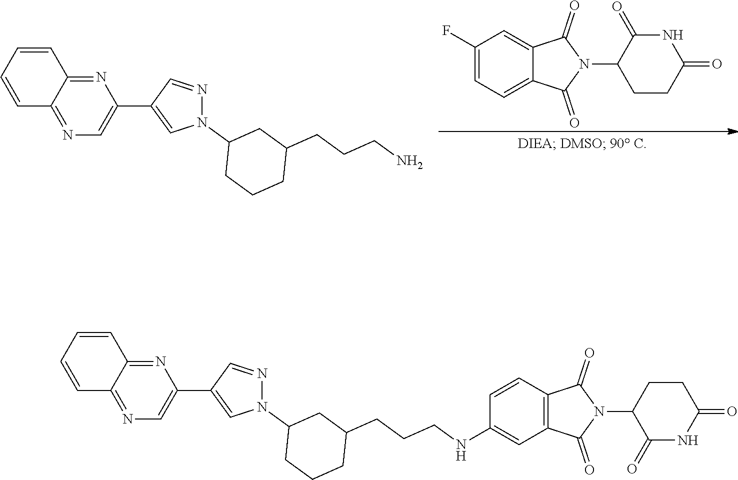 [SMILES: NCCCC1CCCC(n2cc(-c3cnc4ccccc4n3)cn2)C1 ; O=C1CCC(N2C(=O)c3ccc(F)cc3C2=O)C(=O)N1 ; O=C1CCC(N2C(=O)c3ccc(NCCCC4CCCC(n5cc(-c6cnc7ccccc7n6)cn5)C4)cc3C2=O)C(=O)N1]

GS-112 was synthesized following the similar procedure for preparing GS-107 (5.2 mg, yield: 14.7%). MS (ESI) m/z=592.5 [M+H]⁺.

### Example 71. 3-(6-(6-(4-(3-Chlorophenyl)piperidin-1-yl)-6-oxohexyl)-1-oxoisoindolin-2-yl)piperidine-2,6-dione-(GS-113)

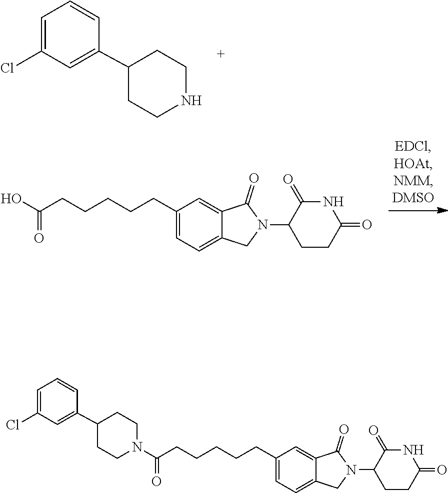 [SMILES: Clc1cccc(C2CCNCC2)c1 ; O=C(O)CCCCCc1ccc2c(c1)C(=O)N(C1CCC(=O)NC1=O)C2 ; O=C1CCC(N2Cc3ccc(CCCCCC(=O)N4CCC(c5cccc(Cl)c5)CC4)cc3C2=O)C(=O)N1]

GS-113 was synthesized following the similar procedure for preparing GS-001 (15 mg, yield: 27.3%). MS (ESI) m/z=536.4 [M+H]⁺.

### Example 72. 3-(6-(6-(4-(4-Chlorophenyl)piperidin-1-yl)-6-oxohexyl)-1-oxoisoindolin-2-yl)piperidine-2,6-dione (GS-114)

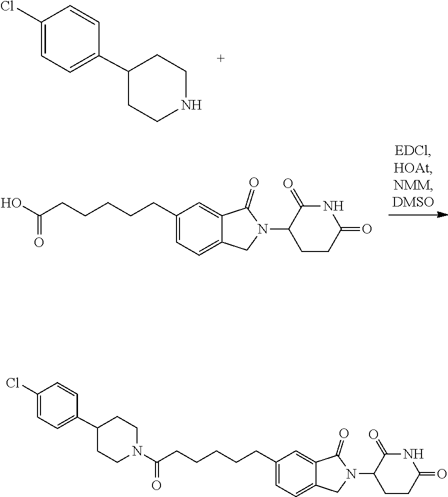 [SMILES: Clc1ccc(C2CCNCC2)cc1 ; O=C(O)CCCCCc1ccc2c(c1)C(=O)N(C1CCC(=O)NC1=O)C2 ; O=C1CCC(N2Cc3ccc(CCCCCC(=O)N4CCC(c5ccc(Cl)cc5)CC4)cc3C2=O)C(=O)N1]

GS-114 was synthesized following the similar procedure for preparing GS-001 (16 mg, yield: 29.2%). MS (ESI) m/z=536.4 [M+H]⁺.

### Example 73. 3-(6-(7-(4-(3-Chlorophenyl)piperidin-1-yl)-7-oxoheptyl)-1-oxoisoindolin-2-yl)piperidine-2,6-dione (GS-115)

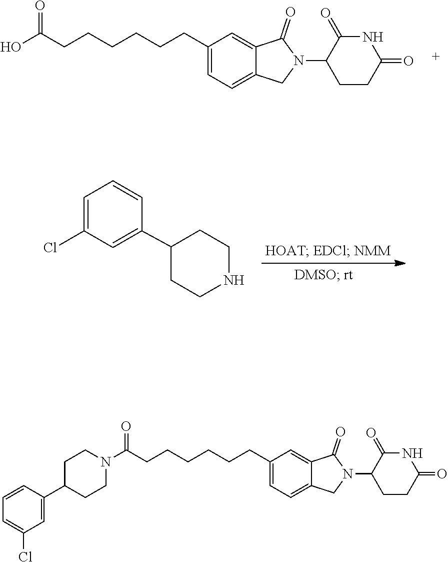 [SMILES: O=C(O)CCCCCCc1ccc2c(c1)C(=O)N(C1CCC(=O)NC1=O)C2 ; Clc1cccc(C2CCNCC2)c1 ; O=C1CCC(N2Cc3ccc(CCCCCCC(=O)N4CCC(c5cccc(Cl)c5)CC4)cc3C2=O)C(=O)N1]

GS-115 was synthesized following the similar procedure for preparing GS-001 (20.68 mg, yield: 69.9%). MS (ESI) m/z=550.5 [M+H]⁺.

### Example 74. 3-(6-(7-(4-(4-Chlorophenyl)piperidin-1-yl)-7-oxoheptyl)-1-oxoisoindolin-2-yl)piperidine-2,6-dione (GS-116)

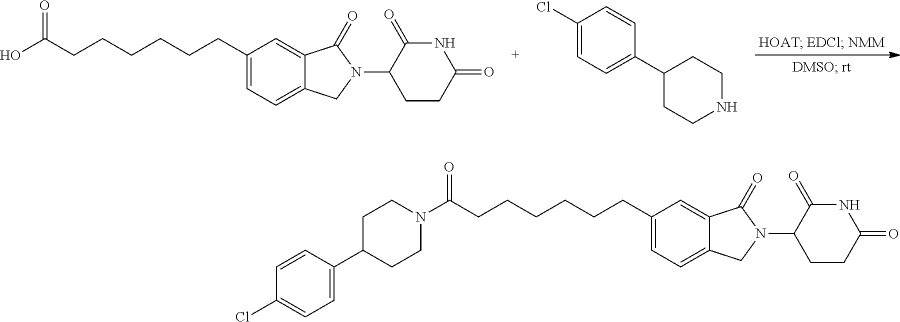 [SMILES: O=C(O)CCCCCCc1ccc2c(c1)C(=O)N(C1CCC(=O)NC1=O)C2 ; Clc1ccc(C2CCNCC2)cc1 ; O=C1CCC(N2Cc3ccc(CCCCCCC(=O)N4CCC(c5ccc(Cl)cc5)CC4)cc3C2=O)C(=O)N1]

GS-116 was synthesized following the similar procedure for preparing GS-001 (24.34 mg, yield: 82.3%). MS (ESI) m/z=550.5 [M+H]⁺.

### Example 75. 2-(2,6-Dioxopiperidin-3-yl)-5-((4-(4-(quinoxalin-2-yl)-1H-pyrazol-1-yl)phenethyl)amino)isoindoline-1,3-dione (GS-117)

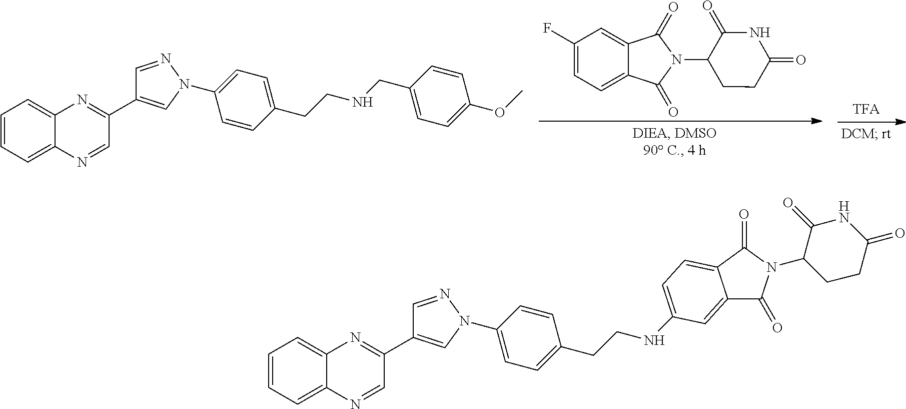 [SMILES: COc1ccc(CNCCc2ccc(-n3cc(-c4cnc5ccccc5n4)cn3)cc2)cc1 ; O=C1CCC(N2C(=O)c3ccc(F)cc3C2=O)C(=O)N1 ; O=C1CCC(N2C(=O)c3ccc(NCCc4ccc(-n5cc(-c6cnc7ccccc7n6)cn5)cc4)cc3C2=O)C(=O)N1]

GS-117 was synthesized following the similar procedure for preparing GS-100 (1.6 mg, yield: 5.1%). MS (ESI) m/z=572.3 [M+H]⁺.

### Example 76. 2-(2,6-Dioxopiperidin-3-yl)-5-((2-(((2-(4-(quinoxalin-2-yl)-1H-pyrazol-1-yl)cyclopropyl)methyl)amino)ethyl)amino)isoindoline-1,3-dione (GS-118)

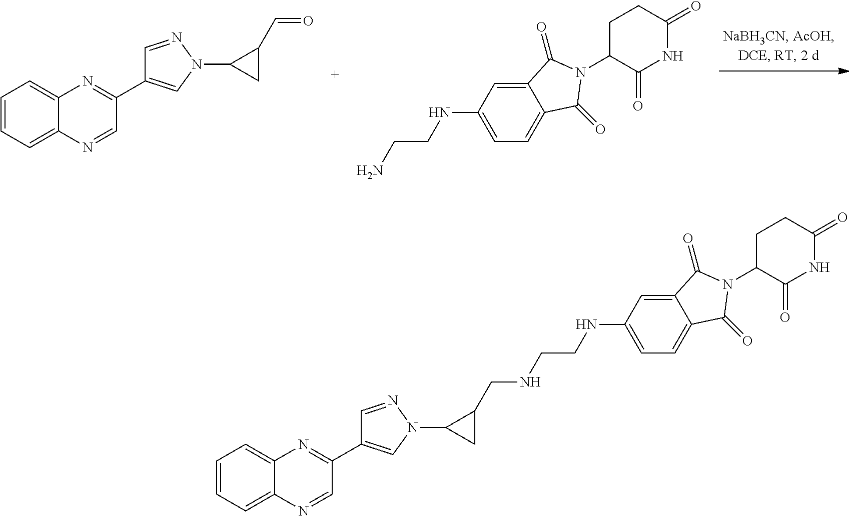 [SMILES: O=CC1CC1n1cc(-c2cnc3ccccc3n2)cn1 ; NCCNc1ccc2c(c1)C(=O)N(C1CCC(=O)NC1=O)C2=O ; O=C1CCC(N2C(=O)c3ccc(NCCNCC4CC4n4cc(-c5cnc6ccccc6n5)cn4)cc3C2=O)C(=O)N1]

To a mixture of 5-((2-aminoethyl)amino)-2-(2,6-dioxopiperidin-3-yl)isoindoline-1,3-dione (11.2 mg, 35.42 μmol), acetic acid (1 drop) and 2-(4-(quinoxalin-2-yl)-1H-pyrazol-1-yl) cyclopropane-1-carbaldehyde (7.8 mg, 29.51 μmol) in DCE (0.5 mL) was added NaBH₃CN (4.6 mg, 73.78 μmol). The reaction mixture was stirred at it for 12 h. Then 2-(4-(quinoxalin-2-yl)-1H-pyrazol-1-yl)cyclopropane-1-carbaldehyde (7.8 mg, 29.51 μmol) and NaBH₃CN (4.6 mg, 73.78 μmol) were added to the reaction mixture and it was stirred at rt for another 12 h. The reaction mixture was diluted with brine and extracted with DCM. The organic layer was concentrated and The resulting residue was purified by prep-TLC (DCM/MeOH=12:1) and prep-HPLC (C18, 0.1% TFA in Acetonitrile) to provide the title compound (1.49 mg, 8.9% yield) as a white solid. MS (ESI) m/z=565.4 [M+H]⁺.

### Example 77. 3-(1-Oxo-4-((3-((2-(4-(4-(quinoxalin-2-yl)-1H-pyrazol-1-yl)piperidin-1-yl)pyrimidin-4-yl)amino)cyclohexyl)amino)isoindolin-2-yl)piperidine-2,6-dione (GS-119)

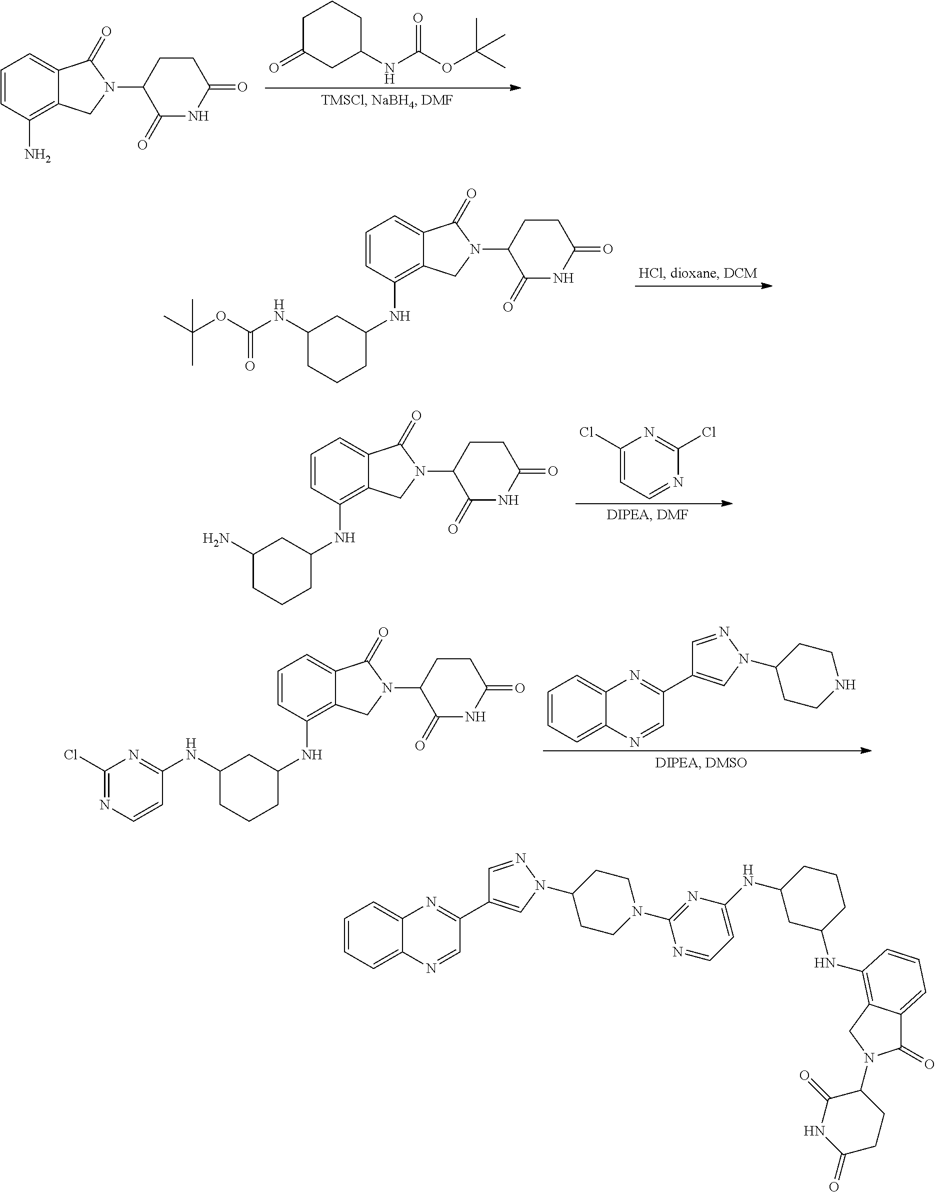

Step 1: Synthesis of tert-butyl (3-((2-(2,6-dioxopiperidin-3-yl)-1-oxoisoindolin-4-yl)amino)cyclohexyl)carbamate

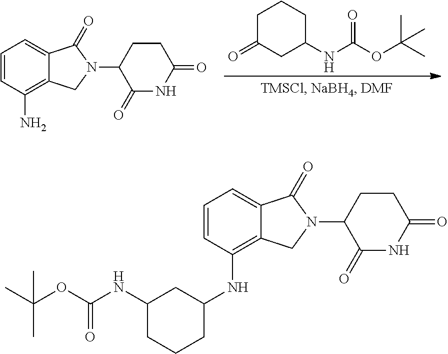

To a solution of tert-butyl N-(3-oxocyclohexyl)carbamate (960 mg, 4.50 mmol) and 3-(4-amino-1-oxoisoindolin-2-yl)piperidine-2,6-dione (777.78 mg, 3.00 mmol) in DMF (10 mL) was added TMSCI (648.00 mg, 6.00 mmol) at 0° C. After completion of addition, the reaction mixture was stirred at 0° C. for 0.5 h, before NaBH₄ (171.00 mg, 4.50 mmol) was added. After the resulting mixture was stirred at 0° C. for 3 h, it was warmed to rt and stirred for 16 h. The reaction was purified by prep-HPLC to provide the title compound (1.3 g, 94.9% yield) as a white solid. MS (ESI) m/z=401.3 [M−56+H]⁺.

Step 2: Synthesis of 3-(4-((3-aminocyclohexyl)amino)-1-oxoisoindolin-2-yl)piperidine-2,6-dione

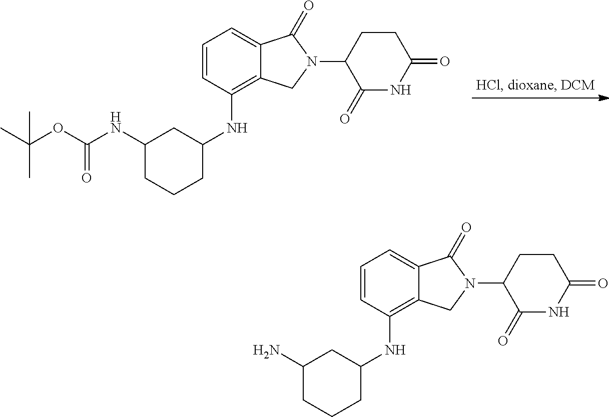 [SMILES: CC(C)(C)OC(=O)NC1CCCC(Nc2cccc3c2CN(C2CCC(=O)NC2=O)C3=O)C1 ; NC1CCCC(Nc2cccc3c2CN(C2CCC(=O)NC2=O)C3=O)C1]

To a solution of tert-butyl N-[3-[[2-(2,6-dioxo-3-piperidyl)-1-oxo-isoindolin-4-yl]amino]cyclohexyl]carbamate (1.3 g, 2.85 mmol) in DCM (20 mL) was added HCl/dioxane (1M, 20 mL). After completion of addition, the reaction mixture was stirred at rt for 2 h. The reaction was concentrated to provide the title compound (1.1 g, 98.3% yield) as a white solid. MS (ESI) m/z=357.2 [M+H]⁺.

Step 3: Synthesis of 3-(4-((3-((2-chloropyrimidin-4-yl)amino)cyclohexyl)amino)-1-oxoisoindolin-2-yl)piperidine-2,6-dione

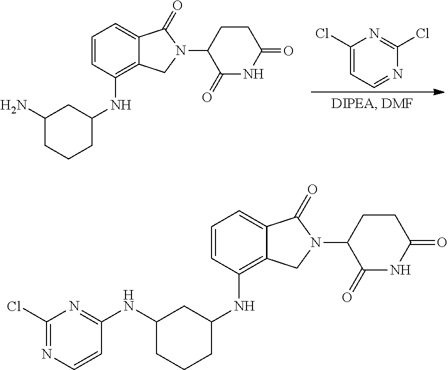 [SMILES: NC1CCCC(Nc2cccc3c2CN(C2CCC(=O)NC2=O)C3=O)C1 ; Clc1ccnc(Cl)n1 ; O=C1CCC(N2Cc3c(NC4CCCC(Nc5ccnc(Cl)n5)C4)cccc3C2=O)C(=O)N1]

To a solution of 3-[4-[(3-aminocyclohexyl)amino]-1-oxo-isoindolin-2-yl]piperidine-2,6-dione (200 mg, 509.06 μmol) and 2,4-dichloropyrimidine (151.68 mg, 1.02 mmol) in DMF (5 mL) was added DIPEA (657.91 mg, 5.09 mmol) at rt. After the mixture was warmed to 80° C. and stirred for 16 h, it was concentrated and purified by silica gel chromatography (DCM/MeOH=30/1 to 20/1) to provide the title compound (200 mg, 83.8% yield). MS (ESI) m/z=469.3 [M+H]⁺.

Step 4: Synthesis of 3-(1-oxo-4-((3-((2-(4-(4-(quinoxalin-2-yl)-1H-pyrazol-1-yl)piperidin-1-yl)pyrimidin-4-yl)amino)cyclohexyl)amino)isoindolin-2-yl)piperidine-2,6-dione

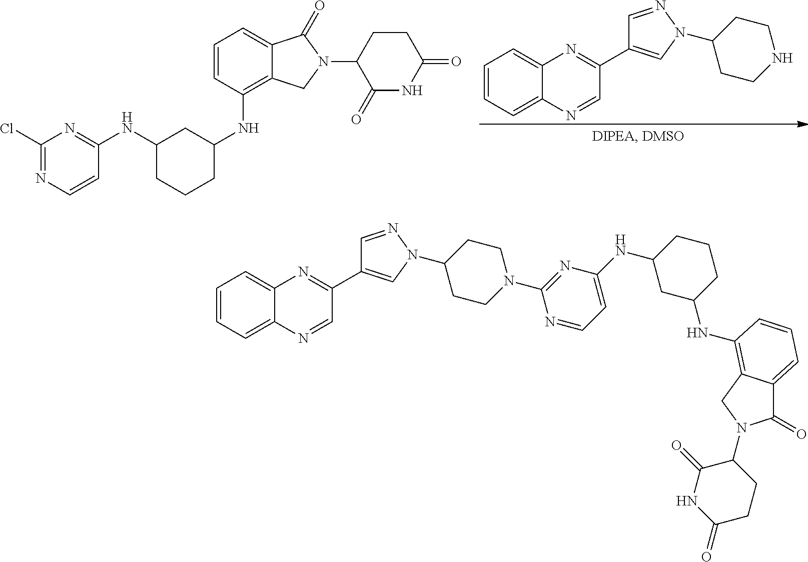

To a solution of 2-[1-(4-piperidyl)pyrazol-4-yl]quinoxaline (28 mg, 100.24 μmol) and 3-[4-[[3-[(2-chloropyrimidin-4-yl)amino]cyclohexyl]amino]-1-oxo-isoindolin-2-yl]piperidine-2,6-dione (47.00 mg, 100.24 μmol) in DMSO (5 mL) was added DIPEA (77.73 mg, 601.42 μmol) at rt. After the mixture was warmed to 100° C. and stirred for 16 h, it was purified by prep-HPLC and prep-TLC (DCM/MeOH=10/1) to provide the title compound (45 mg, 63.1% yield). MS (ESI) m/z=712.7 [M+H]⁺.

### Example 78. N-((2-(2,6-dioxopiperidin-3-yl)-1-oxoisoindolin-5-yl)methyl)-3-(4-(4-(quinoxalin-2-yl)-1H-pyrazol-1-yl)piperidine-1-carbonyl)bicyclo[1.1.1]pentane-1-carboxamide (GS-120)

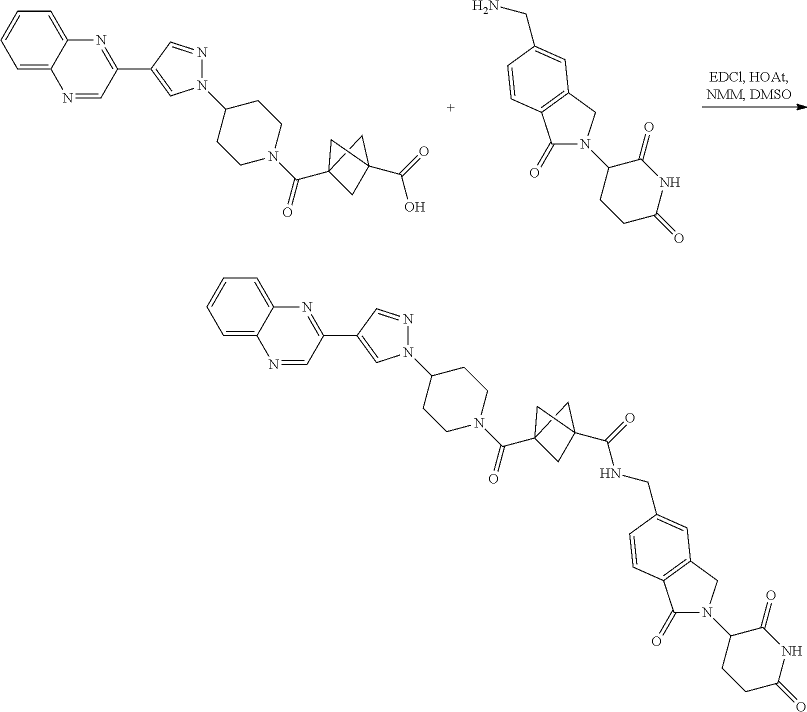

GS-120 was synthesized following the similar procedure for preparing GS-004 (14 mg, yield: 44%). MS (ESI) m/z=673.6 [M+H]⁺.

### Example 79. N-((2-(2,6-dioxopiperidin-3-yl)-1-oxoisoindolin-5-yl)methyl)-2-(3-(4-(4-(quinoxalin-2-yl)-1H-pyrazol-1-yl)piperidine-1-carbonyl)bicyclo[1.1.1]pentan-1-yl)acetamide (GS-121)

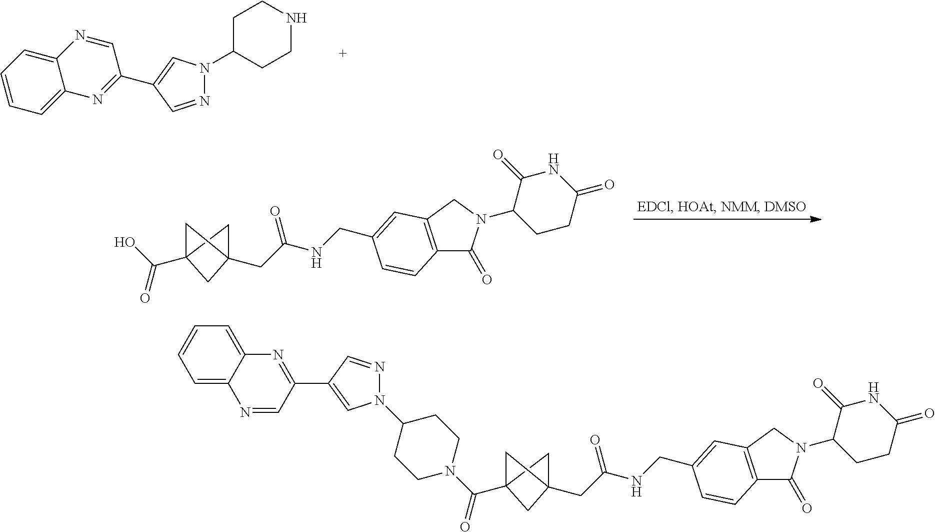

GS-121 was synthesized following the similar procedure for preparing GS-001 (4.7 mg, yield: 36.7%). MS (ESI) m/z=687.6 [M+H]⁺.

### Example 80. N-((2-(2,6-dioxopiperidin-3-yl)-1-oxoisoindolin-5-yl)methyl)-2-(4-(4-(4-(quinoxalin-2-yl)-1H-pyrazol-1-yl)piperidin-1-yl)phenyl)acetamide (GS-122)

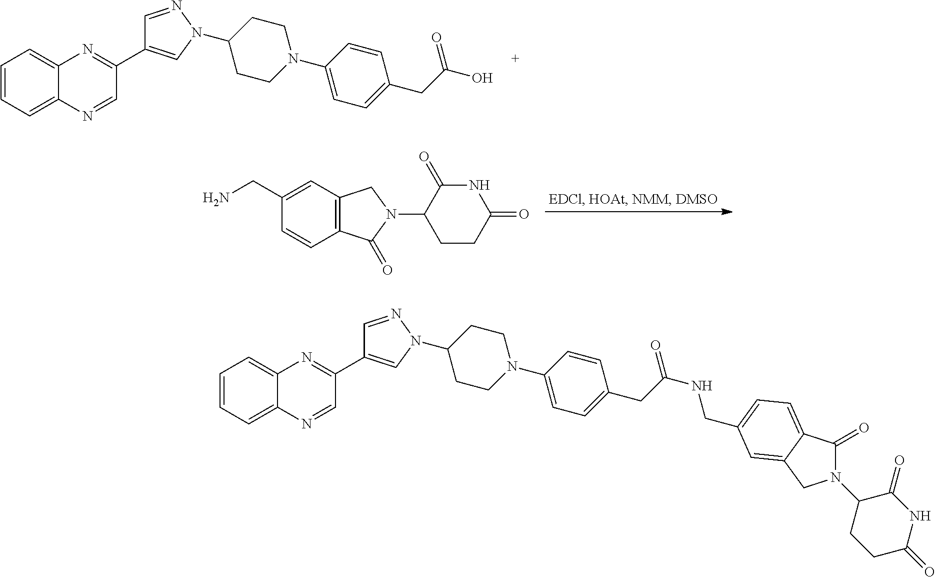

GS-122 was synthesized following the similar procedure for preparing GS-001 (2.2 mg, yield: 45.3%). MS (ESI) m/z=669.5 [M+H]⁺.

### Example 81. 2-(2,6-Dioxopiperidin-3-yl)-5-((5-(1-(4-(4-(quinoxalin-2-yl)-1H-pyrazol-1-yl)piperidin-1-yl)cyclopropyl)pentyl)amino)isoindoline-1,3-dione (GS-123)

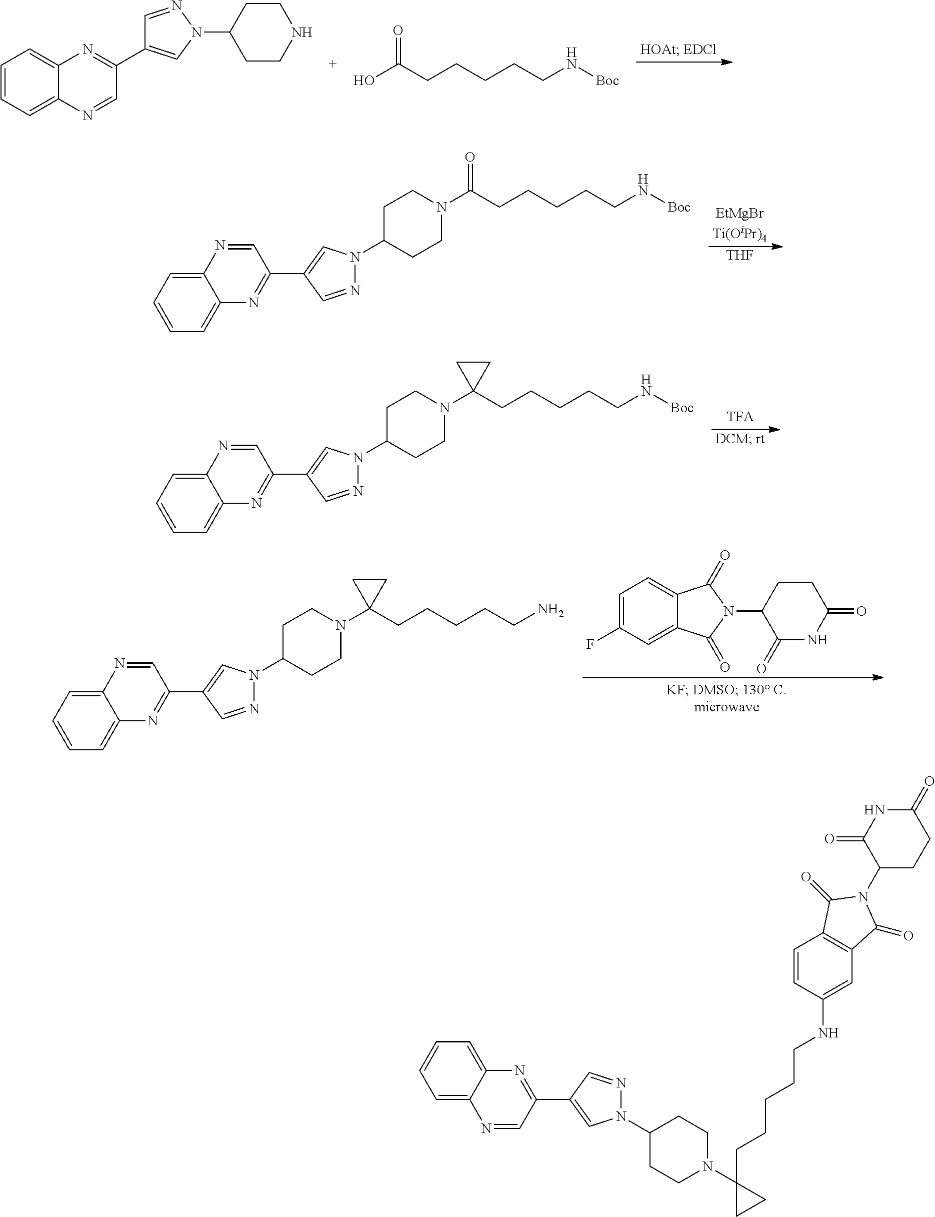

Step 1: Synthesis of tert-butyl (6-oxo-6-(4-(4-(quinoxalin-2-yl)-1H-pyrazol-1-yl)piperidin-1-yl)hexyl)carbamate

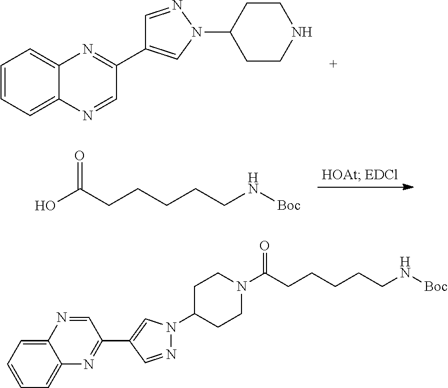

A mixture of 2-(1-(piperidin-4-yl)-1H-pyrazol-4-yl)quinoxaline (250 mg, 0.90 mmol), 6-((tert-butoxycarbonyl)amino)hexanoic acid (207.25 mg, 0.90 mmol), HOAt (182.25 mg, 1.35 mmol), EDCI (259.2 mg, 1.35 mmol) and 4-Methylmorpholine (272.2 mg, 2.7 mmol) in DMSO (5 mL) was stirred at rt for 12 h. Then the mixture was purified by flash chromatography on silica C18 to provide the title compound (400 mg, yield: 90.3%) as a yellow solid. MS (ESI) m/z=493.5 [M+H]⁺.

Step 2: Synthesis of tert-butyl (5-(1-(4-(4-(quinoxalin-2-yl)-1H-pyrazol-1-yl)piperidin-1-yl)cyclopropyl)pentyl)carbamate

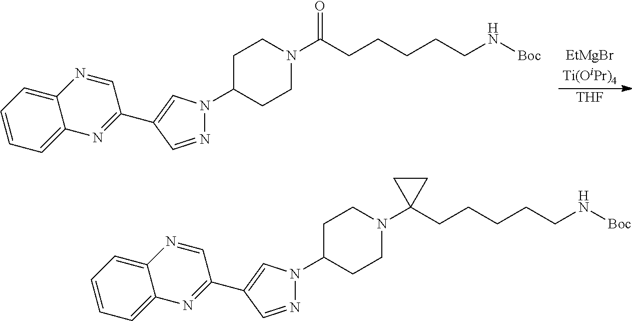 [SMILES: CC(C)(C)OC(=O)NCCCCCC(=O)N1CCC(n2cc(-c3cnc4ccccc4n3)cn2)CC1 ; CC(C)(C)OC(=O)NCCCCCC1(N2CCC(n3cc(-c4cnc5ccccc5n4)cn3)CC2)CC1 ; "THF"]

Under an atmosphere of nitrogen, tert-butyl (6-oxo-6-(4-(4-(quinoxalin-2-yl)-1H-pyrazol-1-yl)piperidin-1-yl)hexyl)carbamate (30 mg, 0.062 mmol) was dissolved in THF (2 ml), Tetraisopropyl titanate (52.8 mg, 0.186 mmol) was added followed by a solution of 3.4 M ethylmagnesium bromide in THF (0.1 ml, 0.31 mmol). The reaction was monitored by TLC, after completion, it was quenched with sat. aq. NH₄Cl and filtered. The filter cake was washed with ethyl acetate (10 mL*2) and the combined organic layers were washed with saturated sodium bicarbonate solution (50 ml), dried over anhydrous sodium sulfate and concentrated. The resulting residue was purified by prep-TLC to provide the title compound (12 mg, 38.4% yield) as a white solid. MS (ESI) m/z=505.3 [M+H]⁺.

Step 3: Synthesis of 5-(1-(4-(4-(quinoxalin-2-yl)-1H-pyrazol-1-yl)piperidin-1-yl)cyclopropyl)pentan-1-amine

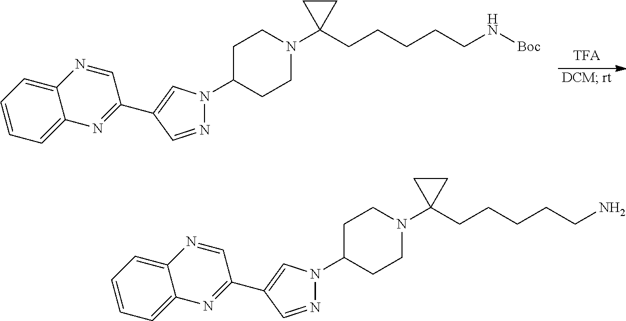 [SMILES: CC(C)(C)OC(=O)NCCCCCC1(N2CCC(n3cc(-c4cnc5ccccc5n4)cn3)CC2)CC1 ; "DCM; rt" ; NCCCCCC1(N2CCC(n3cc(-c4cnc5ccccc5n4)cn3)CC2)CC1]

To a solution of tert-butyl (5-(1-(4-(4-(quinoxalin-2-yl)-1H-pyrazol-1-yl)piperidin-1-yl)cyclopropyl)pentyl)carbamate (50 mg, 0.099 mmol) in DCM (3 mL) was added TFA (1 mL). The mixture was stirred at rt for 1 h, before it was concentrated to provide the title compound (43 mg, yield: 99.9%) as a brown oil which was used directly in the next step without further purification. MS (ESI) m/z=405.3 [M+H]⁺.

Step 4: Synthesis of 2-(2,6-dioxopiperidin-3-yl)-5-((5-(1-(4-(4-(quinoxalin-2-yl)-1H-pyrazol-1-yl)piperidin-1-yl)cyclopropyl)pentyl)amino)isoindoline-1,3-dione

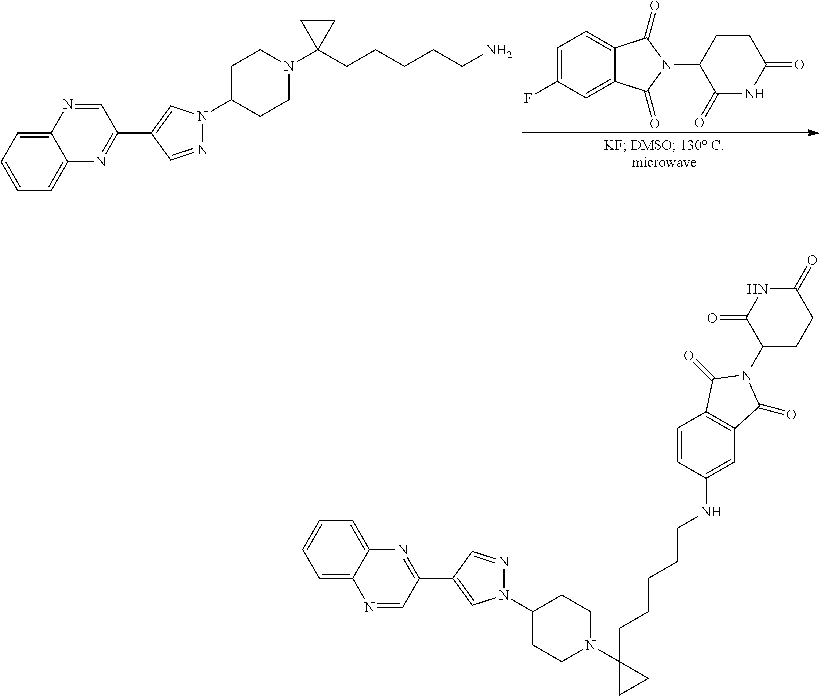 [SMILES: NCCCCCC1(N2CCC(n3cc(-c4cnc5ccccc5n4)cn3)CC2)CC1 ; O=C1CCC(N2C(=O)c3ccc(F)cc3C2=O)C(=O)N1 ; O=C1CCC(N2C(=O)c3ccc(NCCCCCC4(N5CCC(n6cc(-c7cnc8ccccc8n7)cn6)CC5)CC4)cc3C2=O)C(=O)N1]

A mixture of 5-(1-(4-(4-(quinoxalin-2-yl)-1H-pyrazol-1-yl)piperidin-1-yl)cyclopropyl)pentan-1-amine (21 mg, 0.05 mmol), 2-(2,6-dioxopiperidin-3-yl)-5-fluoroisoindoline-1,3-dione (15 mg, 0.057 mmol) and KF (58.1 mg, 1 mmol) in DMSO (0.5 mL) was heated under microwave at 130° C. for 30 min, then it was filtered and the filtrate was purified by flash chromatography on silica C18 to provide the title compound (8.9 mg, yield: 26.9%) as a yellow solid. MS (ESI) m/z=661.6 [M+H]⁺.

### Example 82. 2-(2,6-Dioxopiperidin-3-yl)-5-((4-(1-(4-(4-(quinoxalin-2-yl)-1H-pyrazol-1-yl)piperidin-1-yl)cyclopropyl)butyl)amino)isoindoline-1,3-dione (GS-124)

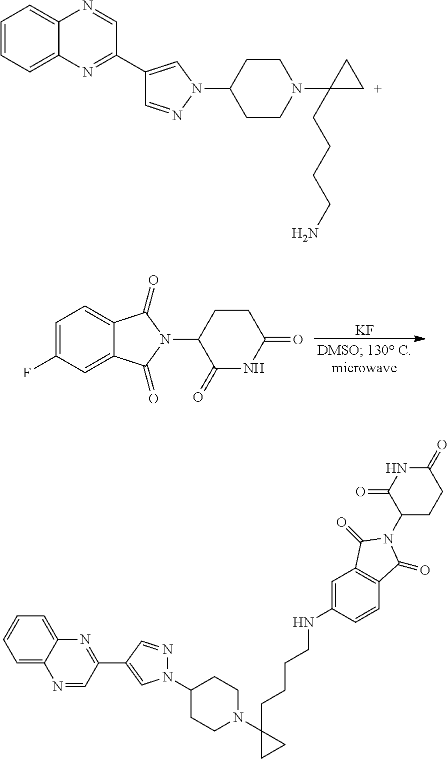

GS-124 was synthesized following the similar procedure for preparing GS-123 (3.9 mg, yield: 23.2%). MS (ESI) m/z=647.7 [M+H]⁺.

### Example 83. 3-(1-Oxo-4-((3-((4-(4-(4-(quinoxalin-2-yl)-1H-pyrazol-1-yl)piperidin-1-yl)pyrimidin-2-yl)amino)cyclohexyl)amino)isoindolin-2-yl)piperidine-2,6-dione (GS-125)

Step 1: Synthesis of tert-butyl (3-((2-(2,6-dioxopiperidin-3-yl)-1-oxoisoindolin-4-yl)amino)cyclohexyl)carbamate

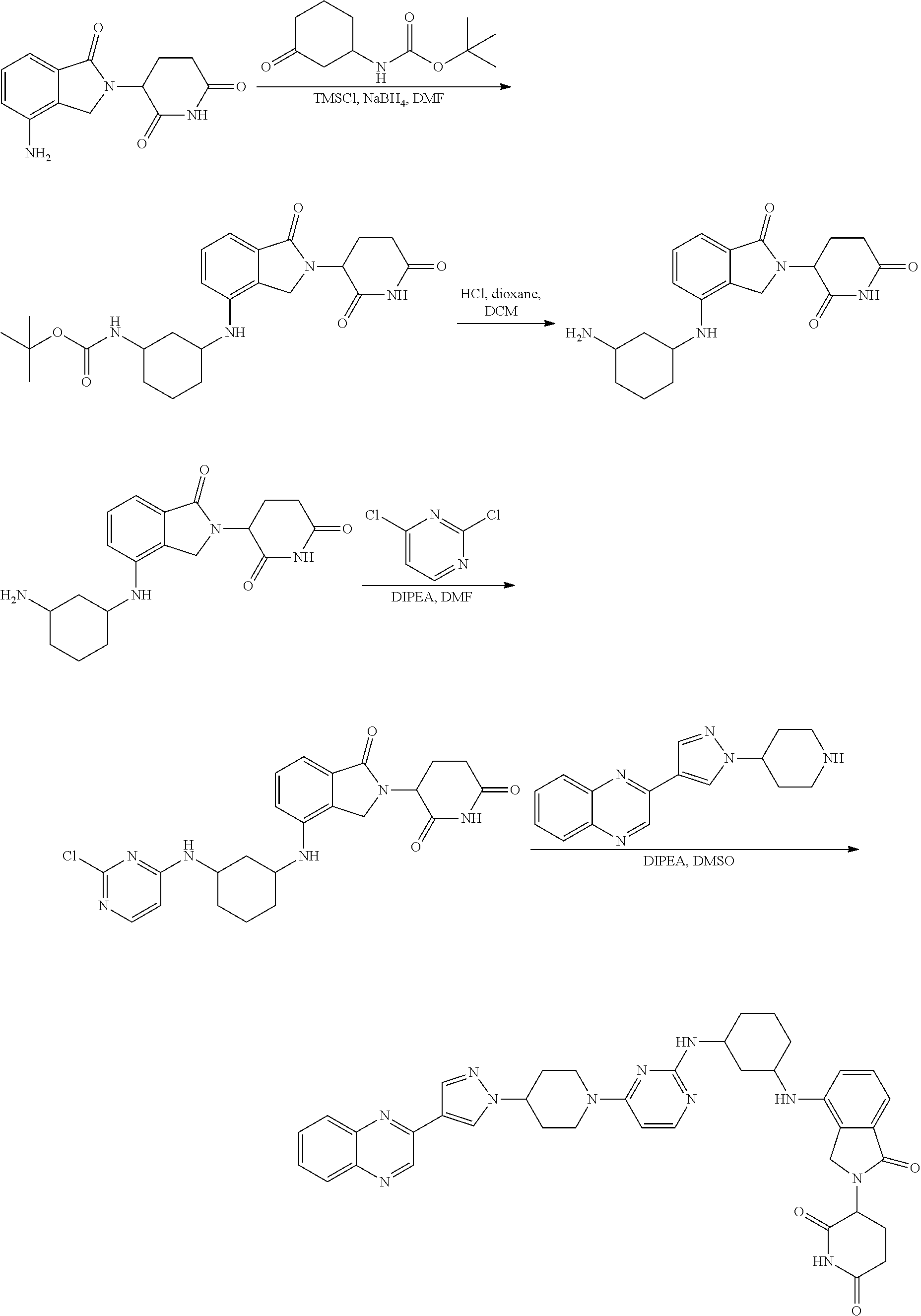

Step 1: synthesis of tert-butyl (3-((2-(2,6-dioxopiperidin-3-yl)-1-oxoisoindolin-4-yl)amino)cyclohexyl)carbamate

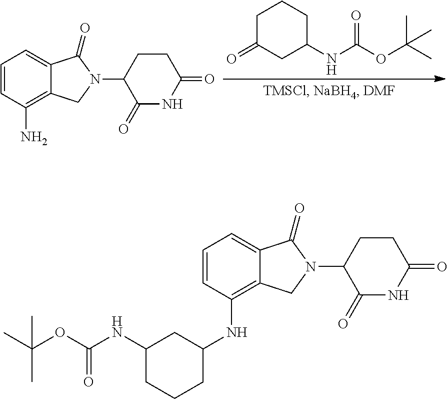

To a solution of tert-butyl N-(3-oxocyclohexyl)carbamate (960 mg, 4.50 mmol) and 3-(4-amino-1-oxo-isoindolin-2-yl)piperidine-2,6-dione (777.78 mg, 3.0 mmol) in DMF (10 mL) was added TMSCI (648 mg, 6.0 mmol) at 0° C. After the completion of addition, the reaction mixture was stirred at 0° C. for 0.5 h, at which time NaBH₄ (171 mg, 4.5 mmol) was added. The mixture was stirred at 0° C. for another 3 h, before it was warmed to rt and stirred for 16 h. The reaction mixture was purified by prep-HPLC to provide the title compound (1.3 g, 94.9% yield) as a white solid. MS (ESI) m/z=401.3 [M−56+H]⁺.

Step 2: Synthesis of 3-(4-((3-aminocyclohexyl)amino)-1-oxoisoindolin-2-yl)piperidine-2,6-dione

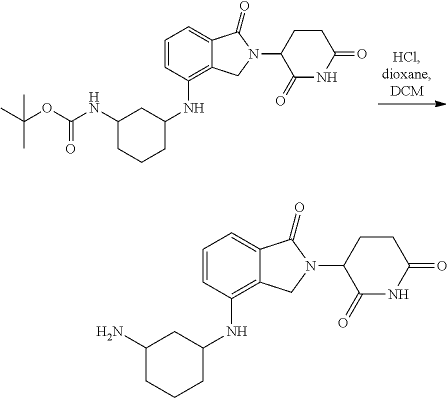 [SMILES: CC(C)(C)OC(=O)NC1CCCC(Nc2cccc3c2CN(C2CCC(=O)NC2=O)C3=O)C1 ; NC1CCCC(Nc2cccc3c2CN(C2CCC(=O)NC2=O)C3=O)C1]

To a solution of tert-butyl (3-((2-(2,6-dioxopiperidin-3-yl)-1-oxoisoindolin-4-yl)amino)cyclohexyl)carbamate (1.3 g, 2.85 mmol) in DCM (20 mL) was added HCl/dioxane (1M, 20 mL). After the reaction mixture was stirred at rt for 2 h, it was concentrated to provide the title compound (1.1 g, 2.80 mmol, 98.3% yield) as a white solid. MS (ESI) m/z=357.2 [M+H]⁺.

Step 3: Synthesis of 3-(4-((3-((2-chloropyrimidin-4-yl)amino)cyclohexyl)amino)-1-oxoisoindolin-2-yl)piperidine-2,6-dione

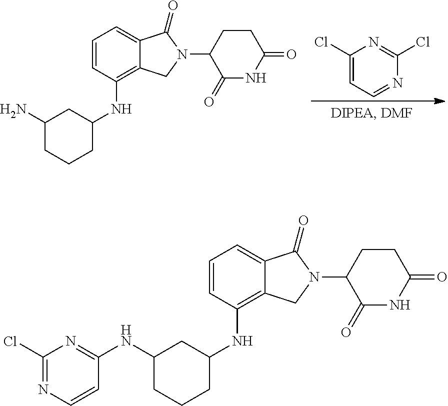

To a solution of 3-(4-((3-aminocyclohexyl)amino)-1-oxoisoindolin-2-yl)piperidine-2,6-dione (200 mg, 509.06 μmol) and 2,4-dichloropyrimidine (151.68 mg, 1.02 mmol) in DMF (5 mL) was added DIPEA (657.91 mg, 5.09 mmol) at rt. After the mixture was warmed to 80° C. and stirred for 16 h, it was concentrated and purified by silica gel chromatography (DCM/MeOH=30/1 to 20/1) to provide the title compound (200 mg, 83.8% yield). MS (ESI) m/z=469.3 [M+H]⁺.

Step 4: Synthesis of 3-(1-oxo-4-((3-((4-(4-(4-(quinoxalin-2-yl)-1H-pyrazol-1-yl)piperidin-1-yl)pyrimidin-2-yl)amino)cyclohexyl)amino)isoindolin-2-yl)piperidine-2,6-dione

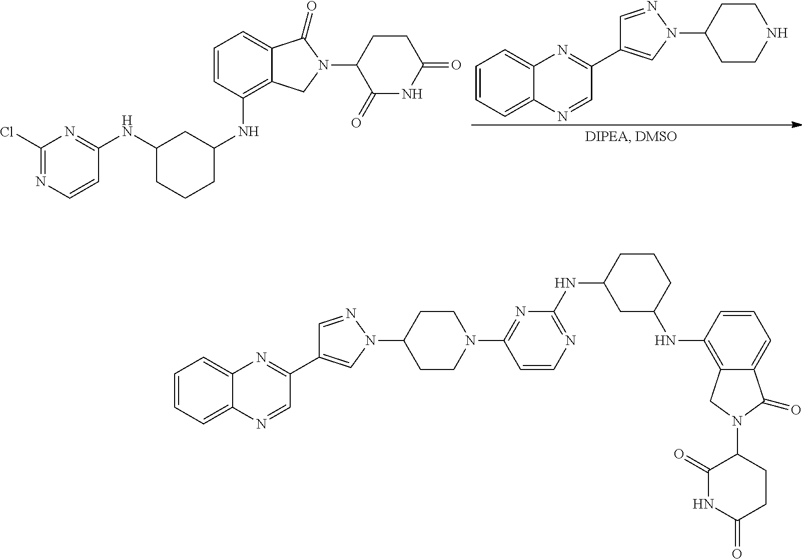

To a solution of 2-(1-(piperidin-4-yl)-1H-pyrazol-4-yl)quinoxaline (28 mg, 100.24 μmol) and 3-(4-((3-((2-chloropyrimidin-4-yl)amino)cyclohexyl)amino)-1-oxoisoindolin-2-yl)piperidine-2,6-dione (47 mg, 100.24 μmol) in DMSO (5 mL) was added DIPEA (77.73 mg, 601.42 μmol) at rt. After the mixture was warmed to 100° C. and stirred for 16 h, it was purified by prep-HPLC and prep-TLC (DCM/MeOH=10/1) to provide the title compound (45 mg, 63.1% yield). MS (ESI) m/z=712.7 [M+H]⁺.

### Example 84. 3-(1-Oxo-5-((5-(1-(4-(4-(quinoxalin-2-yl)-1H-pyrazol-1-yl)piperidin-1-yl)cyclopropyl)pentyl)amino)isoindolin-2-yl)piperidine-2,6-dione (GS-126)

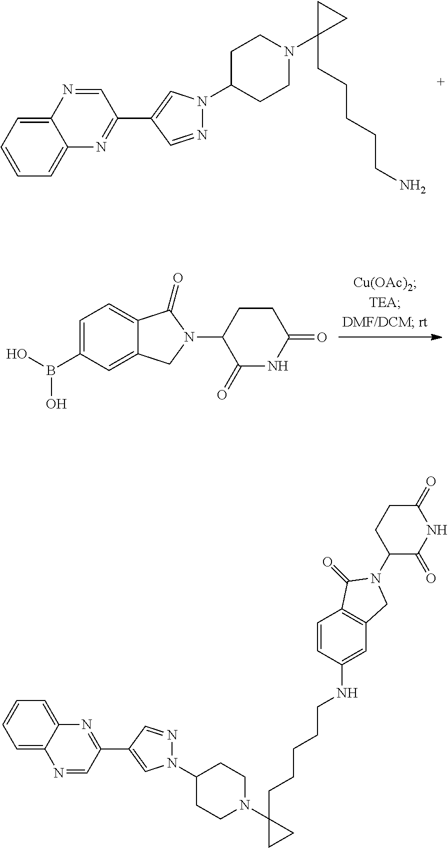 [SMILES: NCCCCCC1(N2CCC(n3cc(-c4cnc5ccccc5n4)cn3)CC2)CC1 ; O=C1CCC(N2Cc3cc(B(O)O)ccc3C2=O)C(=O)N1 ; O=C1CCC(N2Cc3cc(NCCCCCC4(N5CCC(n6cc(-c7cnc8ccccc8n7)cn6)CC5)CC4)ccc3C2=O)C(=O)N1]

To a solution of (2-(2,6-dioxopiperidin-3-yl)-1-oxoisoindolin-5-yl)boronic acid (20 mg, 0.07 mmol) in DCM (0.4 mL) and DMF (0.1 mL) were added 5-(1-(4-(4-(quinoxalin-2-yl)-1H-pyrazol-1-yl)piperidin-1-yl)cyclopropyl)pentan-1-amine (56 mg, 0.14 mmol), Cu(OAc)₂ (14.62 mg, 0.08 mmol), TEA (0.1 mL) and 4 Å molecular sieves (20 mg). The mixture was stirred at rt for 16 h under oxygen. After the reaction was completed, the solid was filtered. The filtrate was concentrated under vacuum and The resulting residue was purified by reverse phase FC to provide the title compound (6.2 mg, yield: 13.7%) as a white solid. MS (ESI) m/z=647.5 [M+H]⁺.

### Example 85. 3-(1-Oxo-6-(7-(4-(quinoxalin-2-yl)-1H-pyrazol-1-yl)octyl)isoindolin-2-yl)piperidine-2,6-dione (GS-127)

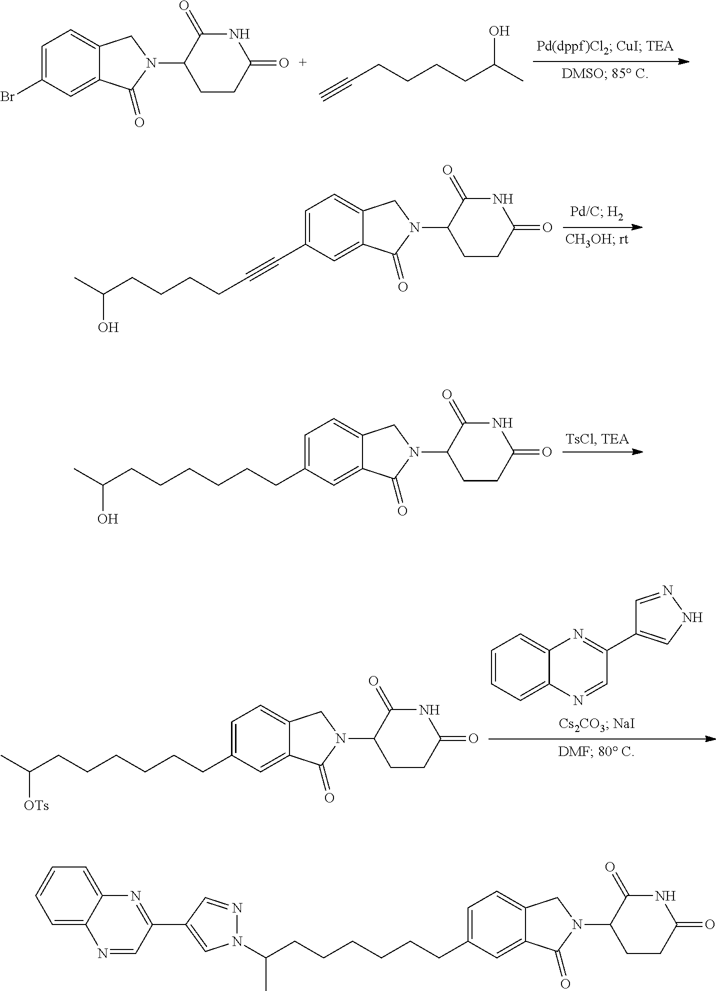

Step 1: Synthesis of 3-(6-(7-hydroxyoct-1-yn-1-yl)-1-oxoisoindolin-2-yl)piperidine-2,6-dione

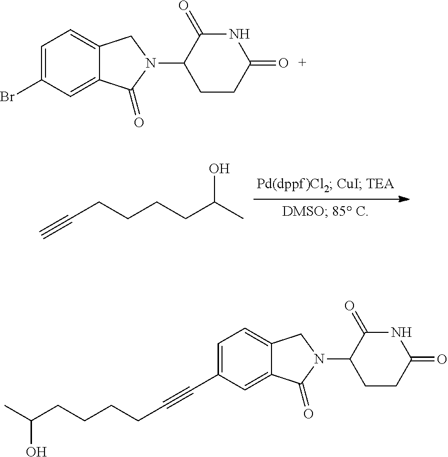

A mixture of 3-(6-bromo-1-oxoisoindolin-2-yl)piperidine-2,6-dione 409 mg, 1.22 mmol), Pd (dppf)Cl₂ (89.18 mg, 0.122 mmol), oct-7-yn-2-ol (230 mg, 1.83 mmol), TEA (1.23 g, 12.2 mmol) and CuI (23.24 mg, 0.122 mmol) in DMSO (5 mL) was stirred at 80° C. for 16 h. The reaction mixture was purified by reverse-phase chromatography to provide the title compound (300 mg, 66.8% yield) as a yellow solid. MS (ESI) m/z=369.3 [M+H]⁺.

Step 2: Synthesis of 3-(6-(7-hydroxyoctyl)-1-oxoisoindolin-2-yl)piperidine-2,6-dione

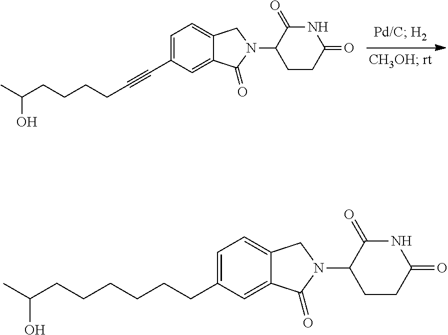 [SMILES: CC(O)CCCCC#Cc1ccc2c(c1)C(=O)N(C1CCC(=O)NC1=O)C2 ; CC(O)CCCCCCc1ccc2c(c1)C(=O)N(C1CCC(=O)NC1=O)C2]

To a solution of 3-(6-(7-hydroxyoct-1-yn-1-yl)-1-oxoisoindolin-2-yl)piperidine-2,6-dione (350 mg, 0.95 mmol) in MeOH (20 mL) was added Pd/C (35 mg). After being stirred at rt for 16 h, the reaction mixture was purified by reverse-phase chromatography to provide the title compound (310 mg, 87.7% yield) as a yellow solid. MS (ESI) m/z=373.2 [M+H]⁺.

Step 3: Synthesis of 8-(2-(2,6-dioxopiperidin-3-yl)-3-oxoisoindolin-5-yl)octan-2-yl 4-methylbenzenesulfonate

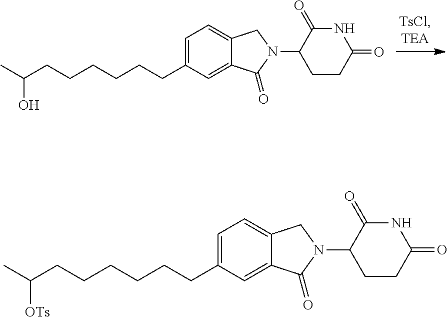 [SMILES: CC(O)CCCCCCc1ccc2c(c1)C(=O)N(C1CCC(=O)NC1=O)C2 ; Cc1ccc(S(=O)(=O)OC(C)CCCCCCc2ccc3c(c2)C(=O)N(C2CCC(=O)NC2=O)C3)cc1]

To a solution of 3-(6-(7-hydroxyoctyl)-1-oxoisoindolin-2-yl)piperidine-2,6-dione (330 mg, 0.89 mmol) in DCM (15 mL) was added TsCl (253.69 mg, 1.33 mmol) and TEA (0.3 mL). After being stirred at rt for 2 h, the reaction mixture was concentrated and The resulting residue was purified by chromatography on silica gel to provide the title compound (180 mg, 38.5% yield) as a yellow solid. MS (ESI) m/z=527.3 [M+H]⁺.

Step 4: Synthesis of 3-(1-oxo-6-(7-(4-(quinoxalin-2-yl)-1H-pyrazol-1-yl)octyl)isoindolin-2-yl)piperidine-2,6-dione

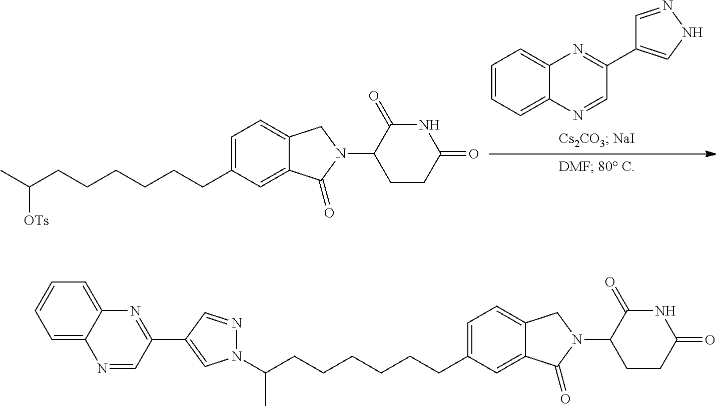 [SMILES: Cc1ccc(S(=O)(=O)OC(C)CCCCCCc2ccc3c(c2)C(=O)N(C2CCC(=O)NC2=O)C3)cc1 ; c1ccc2nc(-c3cn[nH]c3)cnc2c1 ; CC(CCCCCCc1ccc2c(c1)C(=O)N(C1CCC(=O)NC1=O)C2)n1cc(-c2cnc3ccccc3n2)cn1]

To a solution of 2-(1H-pyrazol-4-yl)quinoxaline (13.8 mg, 0.07 mmol) in DMF (1 mL) were added cesium carbonate (68.25 mg, 0.21 mmol), sodium iodide (10.5 mg, 0.07 mmol) and 8-(2-(2,6-dioxopiperidin-3-yl)-3-oxoisoindolin-5-yl)octan-2-yl 4-methylbenzenesulfonate (29.8 mg, 0.07 mmol). The mixture was stirred at 80° C. overnight. The reaction was diluted with water and extracted with ethyl acetate (10 mL*4). The combined organic layers were dried over sodium sulfate and concentrated, The resulting residue was purified by prep-TLC to provide title compound (10.0 mg, yield: 26%) as a yellow solid. MS (ESI) m/z=551.5 [M+H]⁺.

### Example 86. 3-(1-Oxo-6-((6-(4-(quinoxalin-2-yl)-1H-pyrazol-1-yl)hexyl)amino)isoindolin-2-) piperidine-2,6-dione (GS-128)

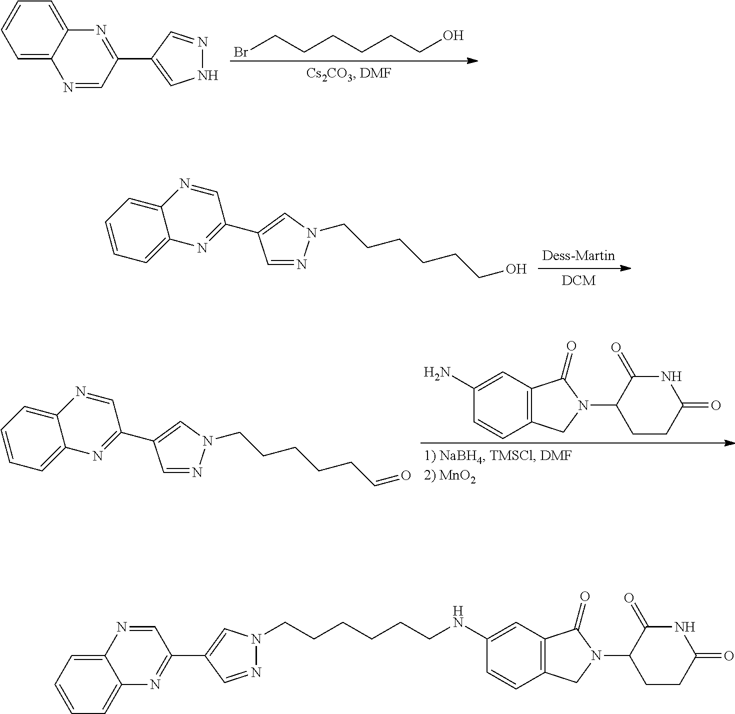

Step 1: Synthesis of 6-(4-(quinoxalin-2-yl)-1H-pyrazol-1-yl)hexan-1-ol

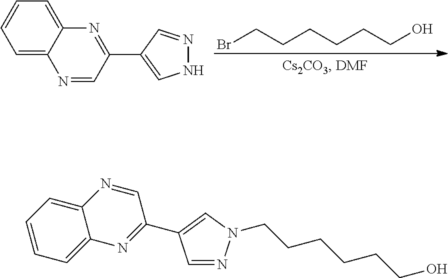

To a solution of 2-(1H-pyrazol-4-yl)quinoxaline (196 mg, 998.94 μmol) and 6-bromohexan-1-ol (271.32 mg, 1.50 mmol) in DMF (5 mL) was added Cs₂CO₃ (973.97 mg, 3.00 mmol) at rt. After the mixture was warmed to 80° C. and stirred for 2 h, it was concentrated and purified by silica gel chromatography (DCM/MeOH=20/1) to provide the title compound (250 mg, 84.4% yield). MS (ESI) m/z=297.2 [M+H]⁺.

Step 2: Synthesis of 6-(4-(quinoxalin-2-yl)-1H-pyrazol-1-yl)hexanal

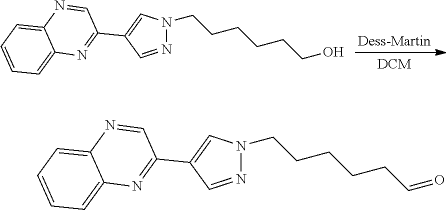 [SMILES: OCCCCCCn1cc(-c2cnc3ccccc3n2)cn1 ; O=CCCCCCn1cc(-c2cnc3ccccc3n2)cn1]

To a solution of 6-(4-quinoxalin-2-ylpyrazol-1-yl)hexan-1-ol (250 mg, 843.55 μmol) in DCM (8 mL) was added Dess-Martin periodinane (715.33 mg, 1.69 mmol) at rt. After completion of addition, the reaction mixture was stirred at rt for 16 h. The reaction was concentrated and purified by silica gel chromatography (DCM/MeOH=20/1) to provide the title compound (180 mg, 72.5% yield). MS (ESI) m/z=295.2 [M+H]⁺.

Step 3: Synthesis of 3-(1-oxo-6-((6-(4-(quinoxalin-2-yl)-1H-pyrazol-1-yl)hexyl)amino)isoindolin-2-yl)piperidine-2,6-dione

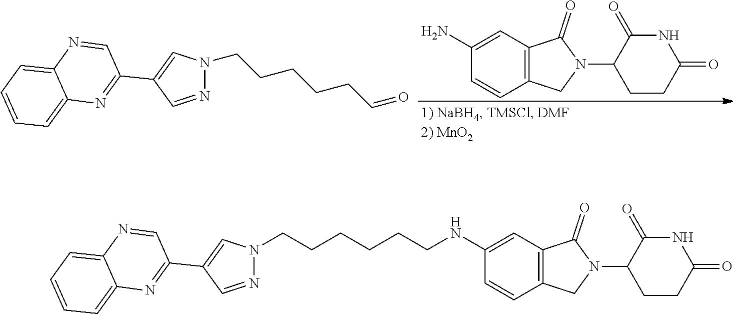 [SMILES: O=CCCCCCn1cc(-c2cnc3ccccc3n2)cn1 ; Nc1ccc2c(c1)C(=O)N(C1CCC(=O)NC1=O)C2 ; O=C1CCC(N2Cc3ccc(NCCCCCCn4cc(-c5cnc6ccccc6n5)cn4)cc3C2=O)C(=O)N1]

To a solution of 6-(4-quinoxalin-2-ylpyrazol-1-yl)hexanal (100 mg, 339.73 μmol) and 3-(6-amino-1-oxo-isoindolin-2-yl)piperidine-2,6-dione (88.08 mg, 339.73 μmol) in DMF (10 mL) was added TMSCI (110.07 mg, 1.02 mmol) at 0° C., followed by NaBH₄ (25.82 mg, 679.46 μmol). After completion of addition, the reaction mixture was stirred at 0° C. for 3 h, before it was warmed to rt and stirred for 10 h. The reaction was purified by prep-HPLC to provide 50 mg crude product which was dissolved in DCM (5 ml). To the resulting solution was added MnO₂ (200 mg). After the mixture was stirred at rt for 0.5 h, it was concentrated and purified by prep-HPLC to provide the title compound (4 mg, 2.2% yield). MS (ESI) m/z=538.4 [M+H]⁺.

### Example 87. 2-(2,6-Dioxopiperidin-3-yl)-5-((4-((4-(4-(quinoxalin-2-yl)-1H-pyrazol-1-yl)piperidin-1-yl)sulfonyl)butyl)amino)isoindoline-1,3-dione (GS-129)

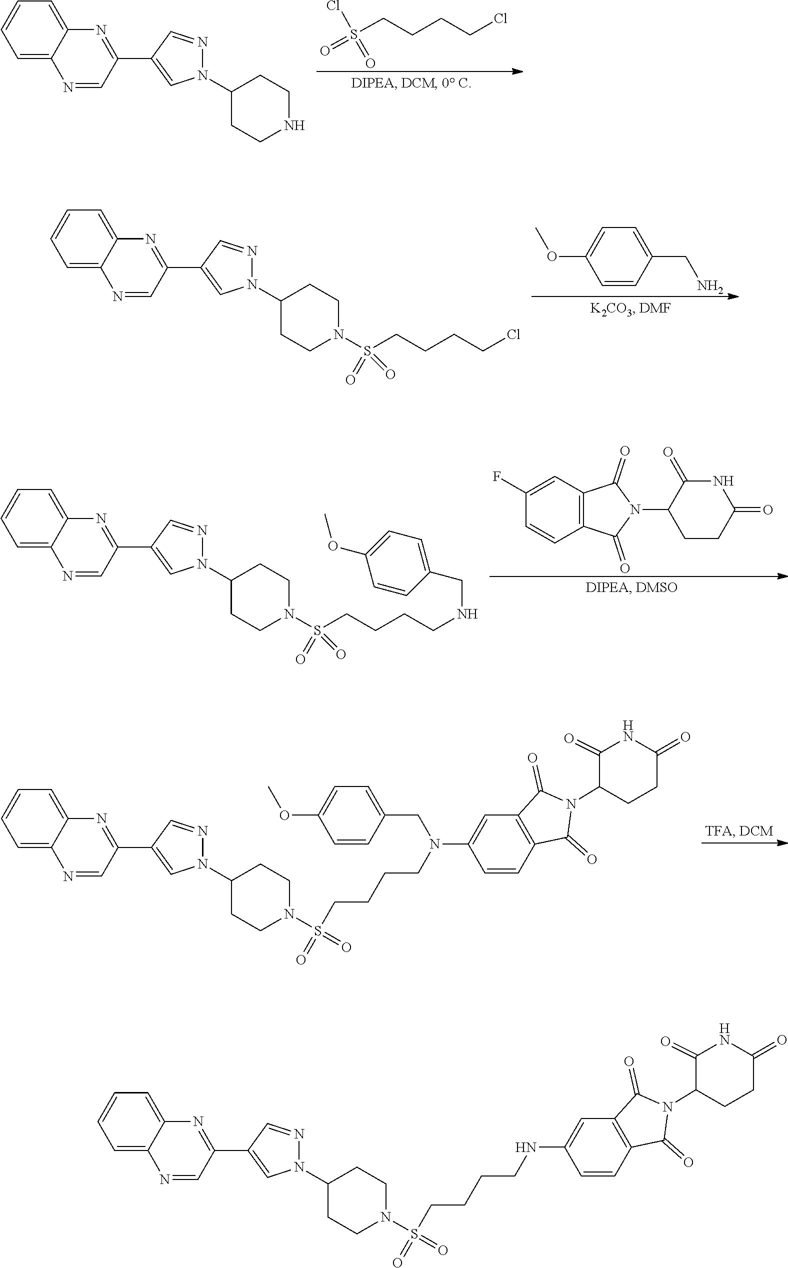

Step 1: Synthesis of 2-(1-(1-((4-chlorobutyl)sulfonyl)piperidin-4-yl)-1H-pyrazol-4-yl)quinoxaline

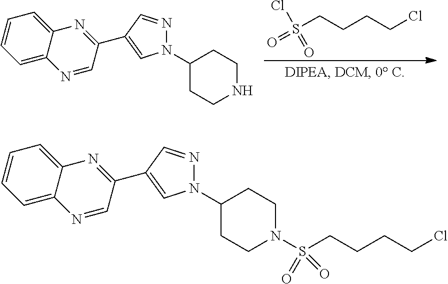 [SMILES: c1ccc2nc(-c3cnn(C4CCNCC4)c3)cnc2c1 ; O=S(=O)(Cl)CCCCCl ; O=S(=O)(CCCCCl)N1CCC(n2cc(-c3cnc4ccccc4n3)cn2)CC1]

To a solution of 2-[1-(4-piperidyl)pyrazol-4-yl]quinoxaline (279 mg, 998.79 μmol) in DCM (10 mL) was added DIPEA (774.50 mg, 5.99 mmol) and 4-chlorobutane-1-sulfonyl chloride (286.27 mg, 1.50 mmol) at 0° C. After completion of addition, the reaction mixture was stirred at 0° C. for 2 h. The reaction was concentrated and purified by silica gel chromatography (DCM/MeOH=30/1) to provide the title compound (400 mg, 92.3% yield) as a yellow solid. MS (ESI) m/z=434.1 [M+H]⁺.

Step 2: Synthesis of N-(4-methoxybenzyl)-4-((4-(4-(quinoxalin-2-yl)-1H-pyrazol-1-yl)piperidin-1-yl)sulfonyl)butan-1-amine

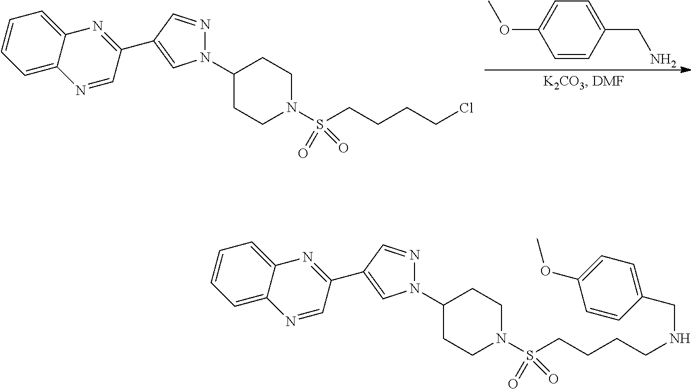

To a solution of 2-[1-[1-(4-chlorobutylsulfonyl)-4-piperidyl]pyrazol-4-yl]quinoxaline (433 mg, 997.80 μmol) and (4-methoxyphenyl)methanamine (410.63 mg, 2.99 mmol) in DMF (10 mL) was added K₂CO₃ (413.09 mg, 2.99 mmol) at rt. After the mixture was warmed to 50° C. and stirred for 4 h, it was concentrated and purified by silica gel chromatography (DCM/MeOH=20/1 to 10/1) to provide the title compound (380 mg, 71.2% yield) as a white solid. MS (ESI) m/z=535.6 [M+H]⁺.

Step 3: Synthesis of 2-(2,6-dioxopiperidin-3-yl)-5-((4-((4-(4-(quinoxalin-2-yl)-1H-pyrazol-1-yl)piperidin-1-yl)sulfonyl)butyl)amino)isoindoline-1,3-dione

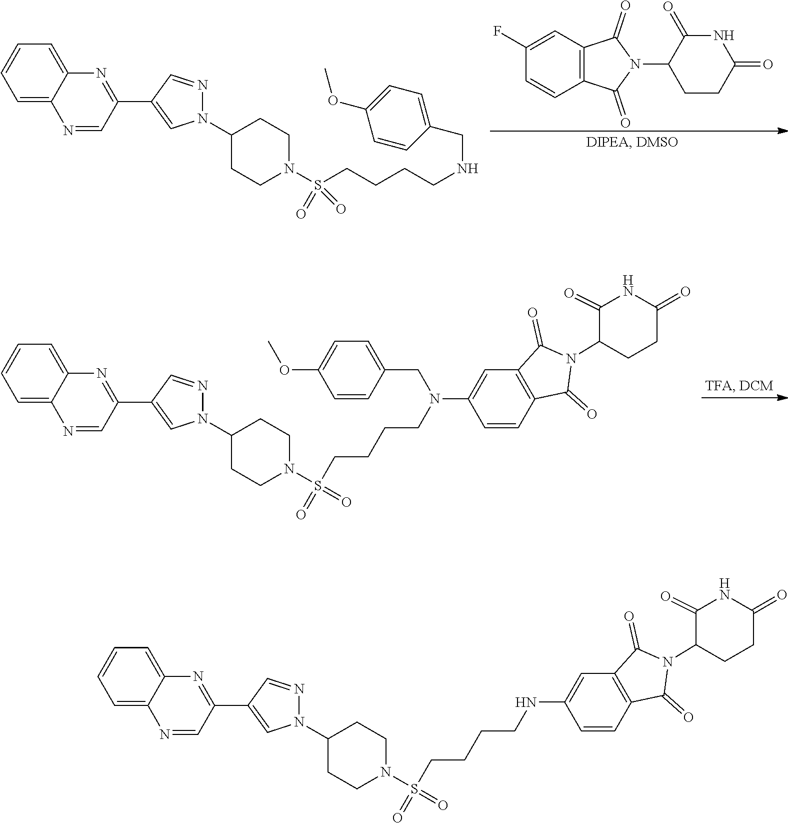 [SMILES: COc1ccc(CNCCCCS(=O)(=O)N2CCC(n3cc(-c4cnc5ccccc5n4)cn3)CC2)cc1 ; O=C1CCC(N2C(=O)c3ccc(F)cc3C2=O)C(=O)N1 ; COc1ccc(CN(CCCCS(=O)(=O)N2CCC(n3cc(-c4cnc5ccccc5n4)cn3)CC2)c2ccc3c(c2)C(=O)N(C2CCC(=O)NC2=O)C3=O)cc1 ; O=C1CCC(N2C(=O)c3ccc(NCCCCS(=O)(=O)N4CCC(n5cc(-c6cnc7ccccc7n6)cn5)CC4)cc3C2=O)C(=O)N1]

To a solution of N-[(4-methoxyphenyl)methyl]-4-[[4-(4-quinoxalin-2-ylpyrazol-1-yl)-1-piperidyl]sulfonyl]butan-1-amine (170 mg, 317.95 μmol) and 2-(2,6-dioxo-3-piperidyl)-5-fluoro-isoindoline-1,3-dione (131.74 mg, 476.93 μmol) in DMSO (10 mL) was added DIPEA (246.55 mg, 1.91 mmol) at rt. After the mixture was warmed to 90° C. and stirred for 2 d, it was purified by prep-HPLC to provide the intermediate 50 mg. The intermediate was dissolved in DCM (10 mL) and TFA (2 mL), and the mixture was stirred at rt for 0.5 h. The reaction was purified by prep-TLC (DCM/MeOH=15/1) to provide the title compound (6 mg, 2.8% yield) as a yellow solid. MS (ESI) m/z=671.4 [M+H]⁺.

### Example 88. N-((2-(2,6-dioxopiperidin-3-yl)-1-oxoisoindolin-5-yl)methyl)-2-(3-(4-(4-(quinoxalin-2-yl)-1H-pyrazol-1-yl)piperidin-1-yl)bicyclo[1.1.1]pentan-1-yl)acetamide (GS-130)

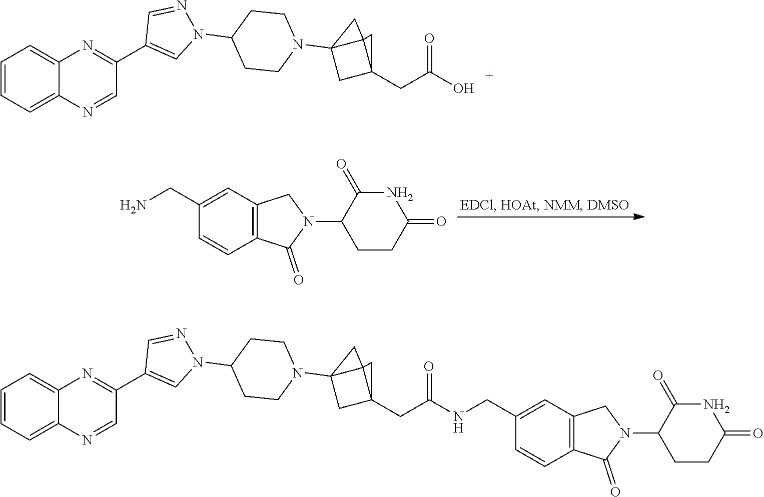 [SMILES: O=C(O)CC12CC3(N4CCC(n5cc(-c6cnc7ccccc7n6)cn5)CC4)CC13C2 ; NCc1ccc2c(c1)CN(C1CCC(=O)NC1=O)C2=O ; O=C(CC12CC3(N4CCC(n5cc(-c6cnc7ccccc7n6)cn5)CC4)CC13C2)NCc1ccc2c(c1)CN(C1CCC(=O)NC1=O)C2=O]

GS-130 was synthesized following the similar procedure for preparing GS-001 (5.1 mg, yield: 31.2%). MS (ESI) m/z=659.5 [M+H]⁺.

### Example 89. 3-(1-Oxo-6-((3-(3-(4-(quinoxalin-2-yl)-1H-pyrazol-1-yl)cyclobutyl)propyl)amino)isoindolin-2-yl)piperidine-2,6-dione (GS-131)

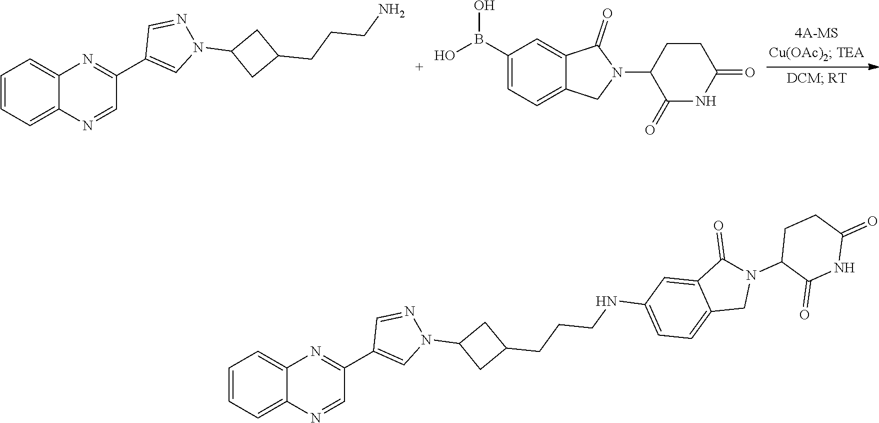 [SMILES: NCCCC1CC(n2cc(-c3cnc4ccccc4n3)cn2)C1 ; O=C1CCC(N2Cc3ccc(B(O)O)cc3C2=O)C(=O)N1 ; O=C1CCC(N2Cc3ccc(NCCCC4CC(n5cc(-c6cnc7ccccc7n6)cn5)C4)cc3C2=O)C(=O)N1]

GS-131 was synthesized following the similar procedure for preparing GS-126 (30.4 mg, yield: 39.6%). MS (ESI) m/z=550.5 [M+H]⁺.

### Example 90. N-(3-((2-(2,6-dioxopiperidin-3-yl)-1,3-dioxoisoindolin-5-yl)amino)propyl)-4-(4-(quinoxalin-2-yl)-1H-pyrazol-1-yl)piperidine-1-carboxamide (GS-132)

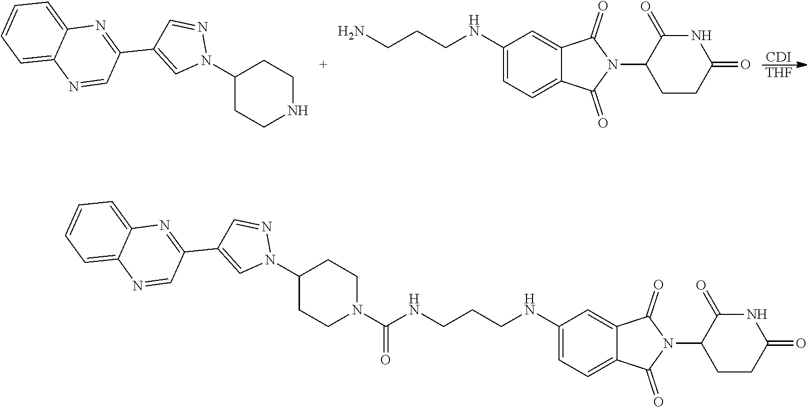

GS-132 was synthesized following the similar procedure for preparing GS-149 (6.63 mg, yield: 10.4%). MS (ESI) m/z=636.4 [M+H]⁺.

### Example 91. 3-(1-Oxo-4-((1-(3-(4-(4-(quinoxalin-2-yl)-1H-pyrazol-1-yl)piperidin-1-yl)benzyl)piperidin-3-yl)amino)isoindolin-2-yl)piperidine-2,6-dione (GS-133)

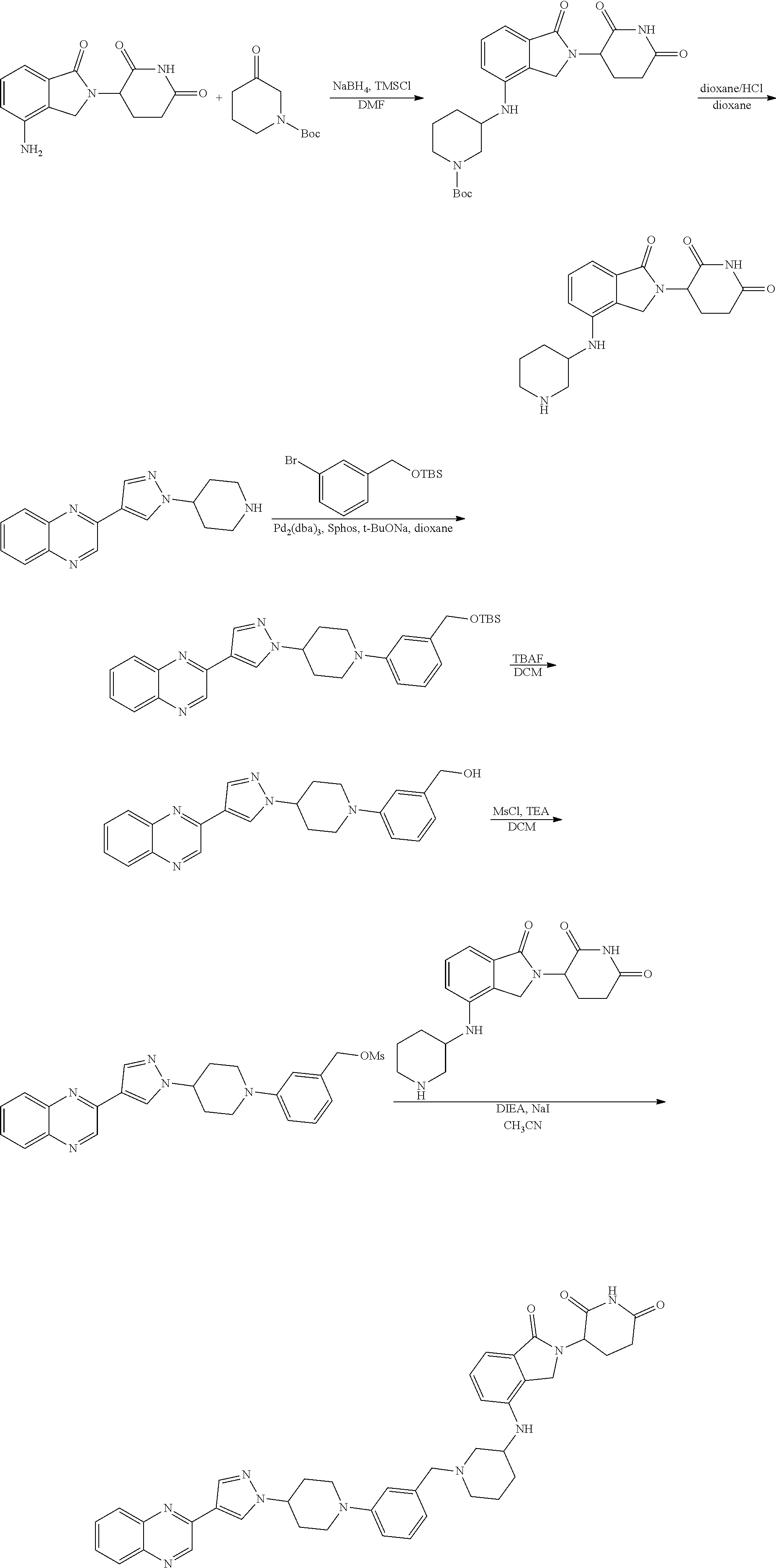

Step 1: Synthesis of tert-butyl 3-((2-(2,6-dioxopiperidin-3-yl)-1-oxoisoindolin-4-yl)amino)piperidine-1-carboxylate

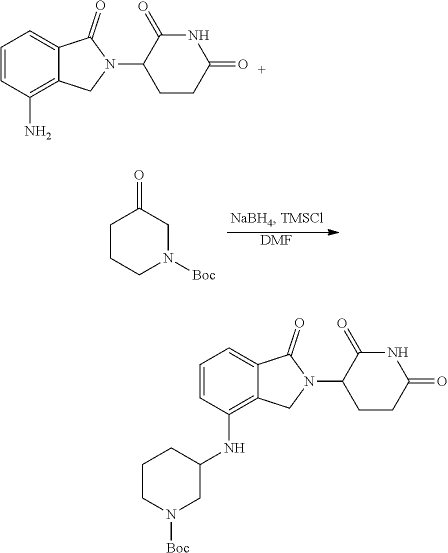

A mixture of 3-(4-amino-1-oxo-isoindolin-2-yl)piperidine-2,6-dione (1.0 g, 3.86 mmol) and tert-butyl 3-oxopiperidine-1-carboxylate (1.15 g, 5.79 mmol) in DMF (15 mL) was stirred at 0° C. for 2 min, before TMSCl (833 mg, 7.71 mmol) was added. After the resulting mixture was stirred at 0° C. for 40 min, NaBH₄ (220 mg, 5.79 mmol) was added. After the reaction was stirred for another 1 h, it was purified by reverse-phase chromatography to provide the title compound (746 mg, 43.7% yield) as a brown solid. MS (ESI) m/z=443.2 [M+H]⁺.

Step 2: Synthesis of 3-(1-oxo-4-(piperidin-3-ylamino)isoindolin-2-yl)piperidine-2,6-dione

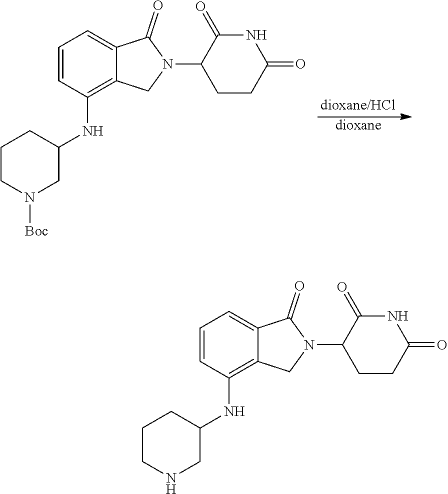 [SMILES: CC(C)(C)OC(=O)N1CCCC(Nc2cccc3c2CN(C2CCC(=O)NC2=O)C3=O)C1 ; "dioxane" ; O=C1CCC(N2Cc3c(NC4CCCNC4)cccc3C2=O)C(=O)N1]

A mixture of tert-butyl 3-[[2-(2,6-dioxo-3-piperidyl)-1-oxo-isoindolin-4-yl]amino]piperidine-1-carboxylate (746 mg, 1.69 mmol) and 4N HCl (4 mL) in dioxane (20 mL) was stirred at 25° C. for 2 h. The reaction was concentrated to provide the title compound (600 mg, 93.9% yield) as a white solid. MS (ESI) m/z=343.1 [M+H]⁺.

Step 3: Synthesis of 2-(1-(1-(3-(((tert-butyldimethylsilyl)oxy)methyl)phenyl)piperidin-4-yl)-1H-pyrazol-4-yl)quinoxaline

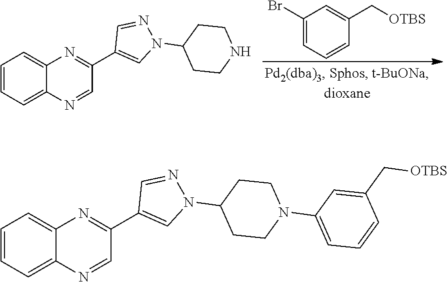 [SMILES: c1ccc2nc(-c3cnn(C4CCNCC4)c3)cnc2c1 ; CC(C)(C)[Si](C)(C)OCc1cccc(Br)c1 ; CC(C)(C)[Si](C)(C)OCc1cccc(N2CCC(n3cc(-c4cnc5ccccc5n4)cn3)CC2)c1]

To a solution of (3-bromophenyl)methoxy-tert-butyl-dimethyl-silane (520 mg, 1.73 mmol) and 2-[1-(4-piperidyl)pyrazol-4-yl]quinoxaline (402 mg, 1.44 mmol) in dioxane (12 mL) were added Pd₂(dba)₃ (264 mg, 0.29 mmol), Sphos (177 mg, 0.43 mmol) and t-BuONa (414 mg, 4.32 mmol). The mixture was stirred at 130° C. for 1 h under N₂. The mixture was purified by silica gel flash chromatography to provide the title compound (700 mg, 97.3% yield) as a brown solid. MS (ESI) m/z=500.6 [M+H]⁺.

Step 4: Synthesis of (3-(4-(4-(quinoxalin-2-yl)-1H-pyrazol-1-yl)piperidin-1-yl)phenyl)methanol

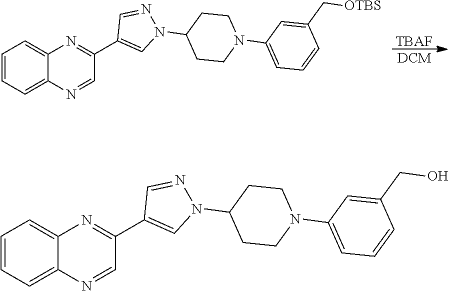

To a solution of tert-butyl-dimethyl-[[3-[4-(4-quinoxalin-2-ylpyrazol-1-yl)-1-piperidyl]phenyl]methoxy]silane (700 mg, 1.40 mmol) in DCM (4 mL) was added TBAF (1N in THF, 2.10 mmol, 2.1 mL). After the reaction mixture was stirred at 25° C. for 5 h, it was purified by reverse-phase chromatography to provide the title compound (500 mg, 92.6% yield) as a brown solid. MS (ESI) m/z=386.5 [M+H]⁺.

Step 5: Synthesis of 3-(4-(4-(quinoxalin-2-yl)-1H-pyrazol-1-yl)piperidin-1-yl)benzyl methanesulfonate

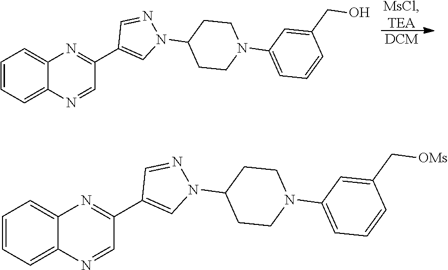

To a solution of [3-[4-(4-quinoxalin-2-ylpyrazol-1-yl)-1-piperidyl]phenyl]methanol (78 mg, 0.20 mmol) and TEA (102.4 mg, 1.01 mmol) in DCM (5 mL) was added MsCl (74 mg, 0.65 mmol) at 0° C. The mixture was stirred at 0° C. for 1 h. The reaction was concentrated to provide the title compound (80 mg, 85.3% yield) as a brown oil which was used in the next step directly without further purification. MS (ESI) m/z=464.3 [M+H]⁺.

Step 6: Synthesis of 3-(1-oxo-4-((1-(3-(4-(4-(quinoxalin-2-yl)-1H-pyrazol-1-yl)piperidin-1-yl)benzyl)piperidin-3-yl)amino)isoindolin-2-yl)piperidine-2,6-dione

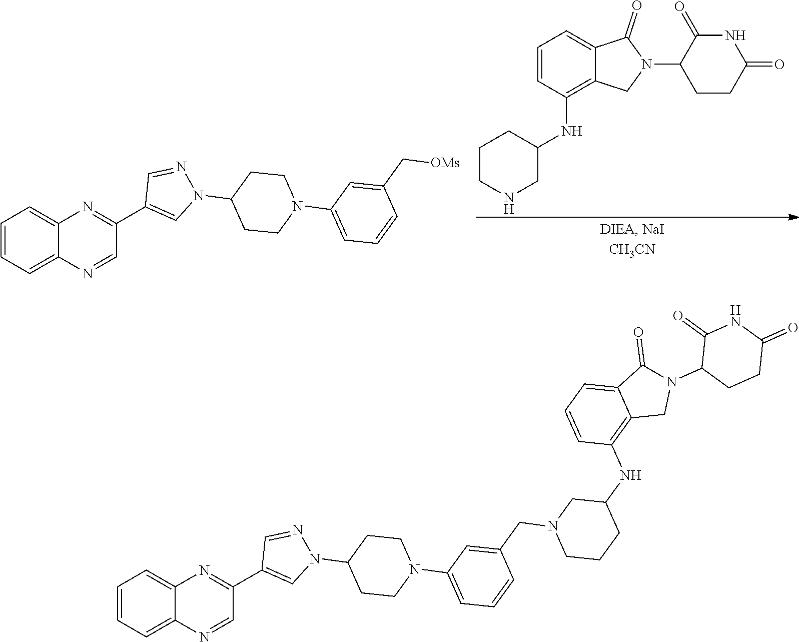

A mixture of [3-[4-(4-quinoxalin-2-ylpyrazol-1-yl)-1-piperidyl]phenyl]methyl methanesulfonate (80 mg, 0.17 mmol), DIEA (44.53 mg, 0.35 mmol), NaI (77.60 mg, 0.52 mmol) and 3-[1-oxo-4-(3-piperidylamino)isoindolin-2-yl]piperidine-2,6-dione (59.09 mg, 0.17 mmol) in CH₃CN (3.0 mL) was stirred at 70° C. for 12 h. The mixture was purified by reserve phase column to provide the title compound (29 mg, 23.7% yield) as a yellow solid. MS (ESI) m/z=710.6 [M+H]⁺.

### Example 92. 3-(6-(7-(4-(3-Methylquinoxalin-2-yl)-1H-pyrazol-1-yl)heptyl)-1-oxoisoindolin-2-yl)piperidine-2,6-dione (GS-134)

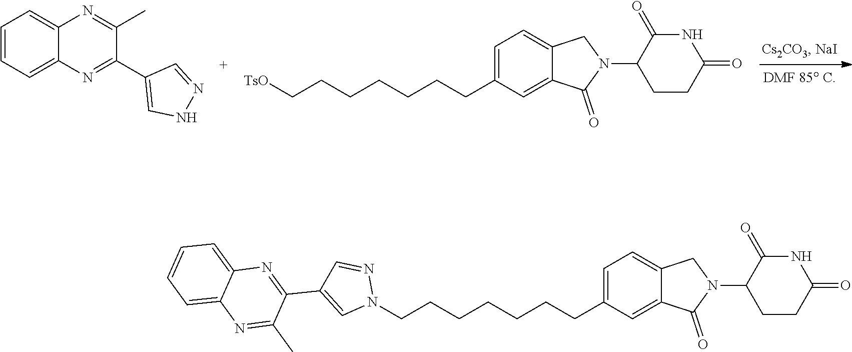 [SMILES: Cc1nc2ccccc2nc1-c1cn[nH]c1 ; Cc1ccc(S(=O)(=O)OCCCCCCCc2ccc3c(c2)C(=O)N(C2CCC(=O)NC2=O)C3)cc1 ; Cc1nc2ccccc2nc1-c1cnn(CCCCCCCc2ccc3c(c2)C(=O)N(C2CCC(=O)NC2=O)C3)c1]

GS-134 was synthesized following the similar procedure for preparing GS-105 (7.2 mg, yield: 33.5%). MS (ESI) m/z=551.4 [M+H]⁺.

### Example 93. 3-(4-((3-((3-(4-(3-Chlorophenyl)piperidin-1-yl)phenyl)amino)cyclohexyl)amino)-1-oxoisoindolin-2-yl)piperidine-2,6-dione (GS-135)

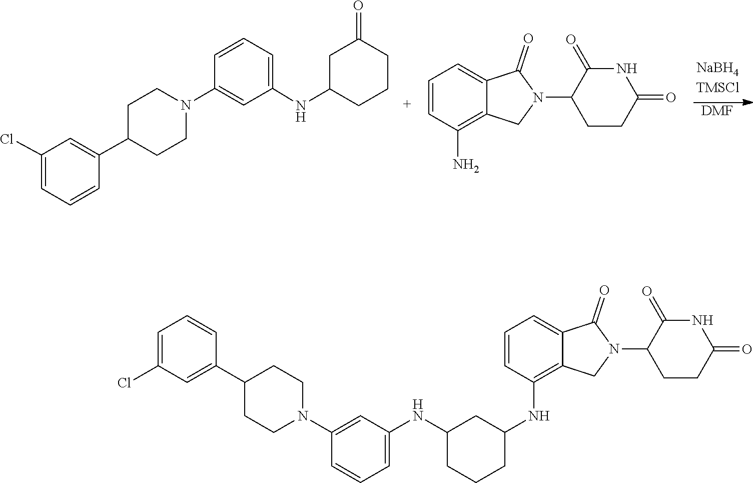

GS-135 was synthesized following the similar procedure for preparing GS-110 (12.8 mg, yield: 15.7%). MS (ESI) m/z=626.5 [M+H]⁺.

### Example 94. 2-(2,6-Dioxopiperidin-3-yl)-5-((5-oxo-5-(3-(4-(quinoxalin-2-yl)-1H-pyrazol-1-yl)-8-azabicyclo[3.2.1]octan-8-yl)pentyl)amino)isoindoline-1,3-dione (GS-136)

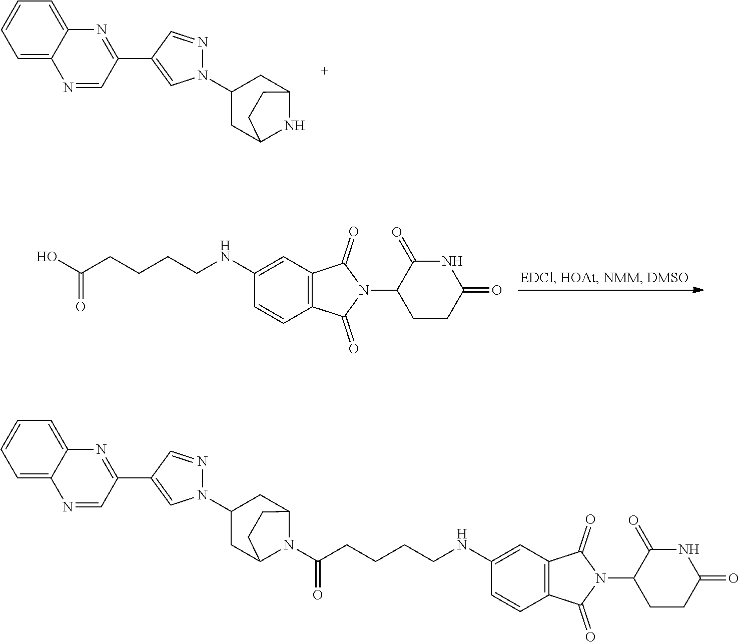 [SMILES: c1ccc2nc(-c3cnn(C4CC5CCC(C4)N5)c3)cnc2c1 ; O=C(O)CCCCNc1ccc2c(c1)C(=O)N(C1CCC(=O)NC1=O)C2=O ; O=C1CCC(N2C(=O)c3ccc(NCCCCC(=O)N4C5CCC4CC(n4cc(-c6cnc7ccccc7n6)cn4)C5)cc3C2=O)C(=O)N1]

GS-136 was synthesized following the similar procedure for preparing GS-001 (10.6 mg, yield: 33.1%). MS (ESI) m/z=661.5 [M+H]⁻.

### Example 95. 3-(1-Oxo-5-((4-(1-(4-(4-(quinoxalin-2-yl)-1H-pyrazol-1-yl)piperidin-1-yl)cyclopropyl)butyl)amino)isoindolin-2-yl)piperidine-2,6-dione (GS-137)

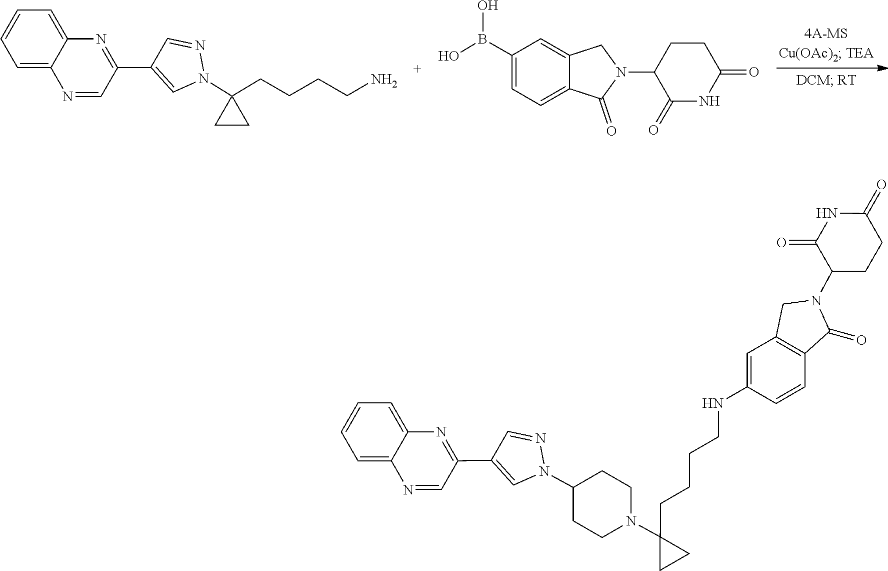 [SMILES: NCCCCC1(n2cc(-c3cnc4ccccc4n3)cn2)CC1 ; O=C1CCC(N2Cc3cc(B(O)O)ccc3C2=O)C(=O)N1 ; O=C1CCC(N2Cc3cc(NCCCCC4(N5CCC(n6cc(-c7cnc8ccccc8n7)cn6)CC5)CC4)ccc3C2=O)C(=O)N1]

GS-137 was synthesized following the similar procedure for preparing GS-126 (6.1 mg, yield: 31.7%). MS (ESI) m/z=633.5 [M+H]⁻.

### Example 96. 2-(2,6-Dioxopiperidin-3-yl)-5-((4-methyl-5-oxo-5-(4-(4-(quinoxalin-2-yl)-1H-pyrazol-1-yl)piperidin-1-yl)pentyl)amino)iaoindoline-1,3-dione (GS-138)

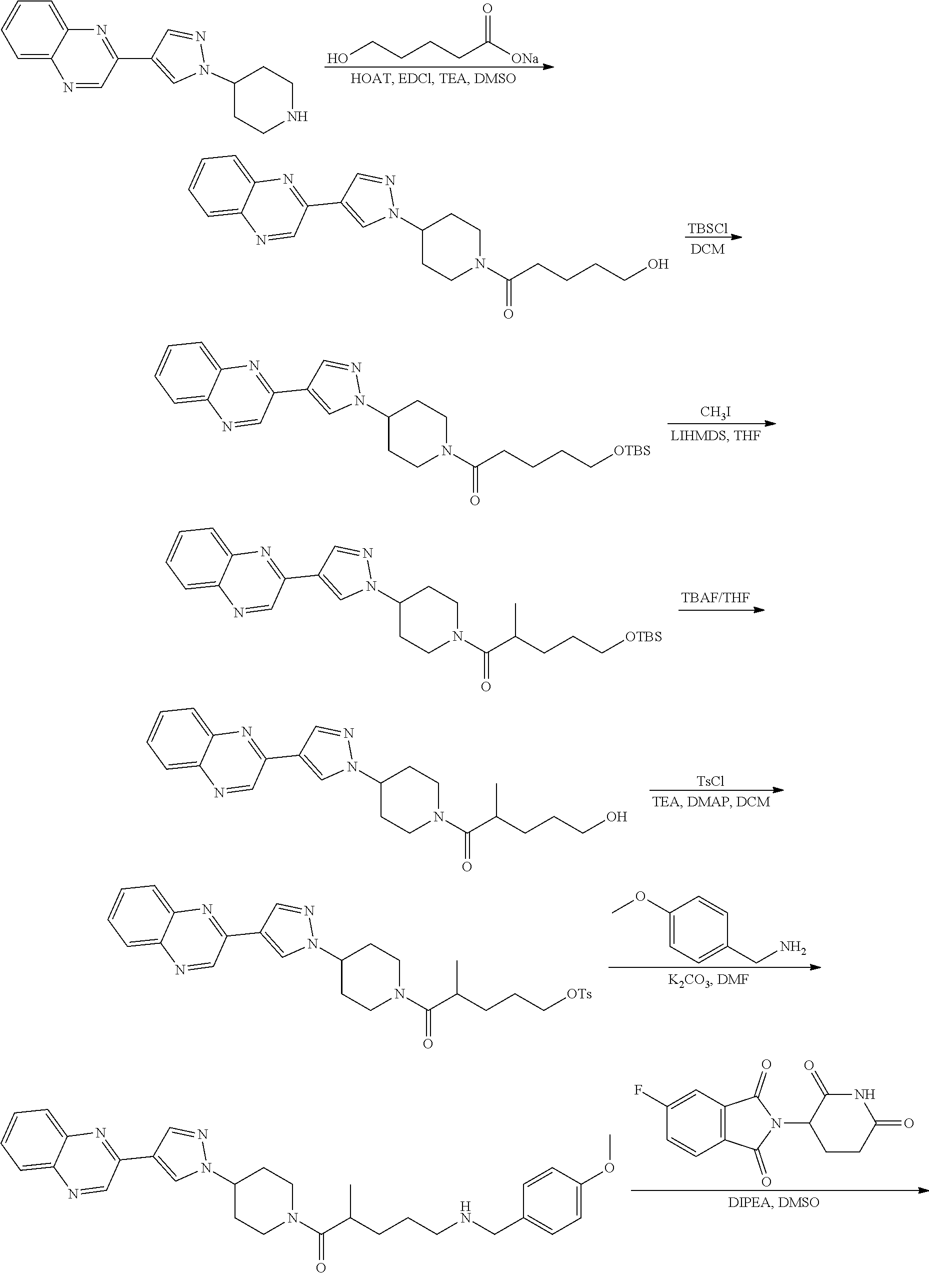

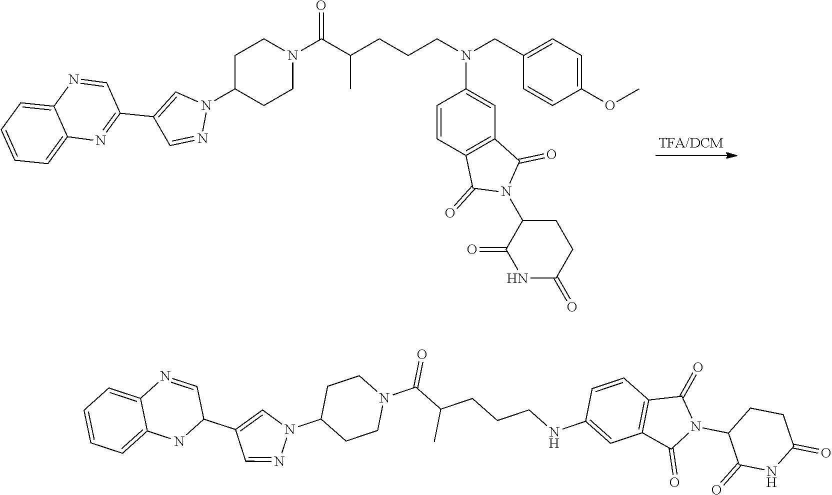

Step 1: Synthesis of 5-hydroxy-1-(4-(4-(quinoxalin-2-yl)-1H-pyrazol-1-yl)piperidin-1-yl)pentan-1-one

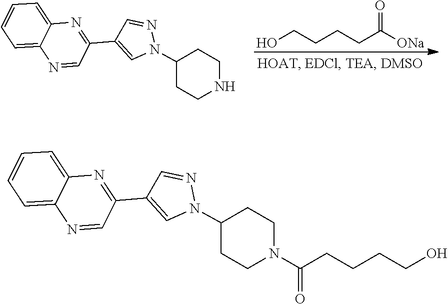

To a solution of 2-[1-(4-piperidyl)pyrazol-4-yl]quinoxaline (1.0 g, 3.58 mmol) and 5-hydroxypentanoyloxysodium (601.91 mg, 4.30 mmol) in DMSO (25 mL) were added HOAt (966.57 mg, 7.16 mmol), EDCI (1.37 g, 7.16 mmol) and TEA (1.45 g, 14.32 mmol) at rt. After completion of addition, the reaction mixture was stirred at it for 16 h. The reaction was quenched with water (50 mL) and extracted with DCM (30 mL×3), dried over Na₂SO₄, concentrated and purified by silica gel chromatography (DCM/MeOH=15/1) to provide the title compound (1.0 g, 73.6% yield) as a white solid. MS (ESI) m/z=380.3 [M+H]⁺.

Step 2: Synthesis of 5-((tert-butyldimethylsilyl)oxy)-1-(4-(4-(quinoxalin-2-yl)-1H-pyrazol-1-yl)piperidin-1-yl)pentan-1-one

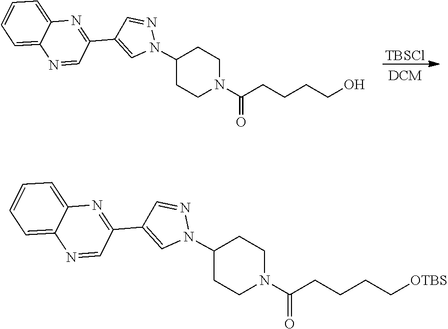 [SMILES: O=C(CCCCO)N1CCC(n2cc(-c3cnc4ccccc4n3)cn2)CC1 ; "TBSCl" ; CC(C)(C)[Si](C)(C)OCCCCC(=O)N1CCC(n2cc(-c3cnc4ccccc4n3)cn2)CC1]

To a solution of 5-hydroxy-1-[4-(4-quinoxalin-2-ylpyrazol-1-yl)-1-piperidyl]pentan-1-one (1.0 g, 2.64 mmol) in DCM (20 mL) were added imidizale (537.61 mg, 7.91 mmol) and TBSCl (595.72 mg, 3.95 mmol) at rt. After completion of addition, the reaction mixture was stirred at rt for 2 h. The reaction was concentrated and purified by silica gel chromatography (DCM/MeOH=20/1) to provide the title compound (1.1 g, 84.5% yield) as a yellow solid. MS (ESI) m/z=494.5 [M+H]⁺.

Step 3: Synthesis of 5-((tert-butyldimethylsilyl)oxy)-2-methyl-1-(4-(4-(quinoxalin-2-yl)-1H-pyrazol-1-yl)piperidin-1-yl)pentan-1-one

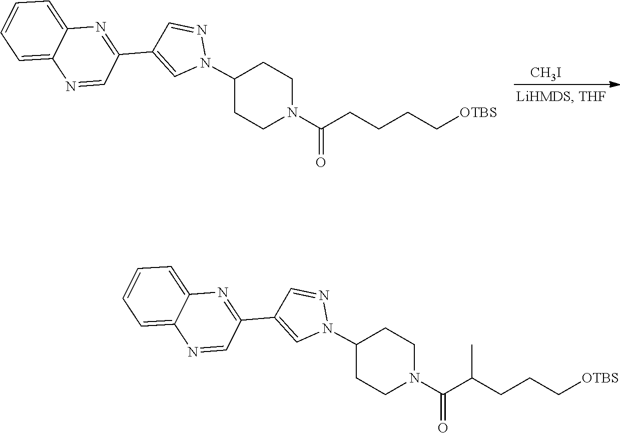 [SMILES: CC(C)(C)[Si](C)(C)OCCCCC(=O)N1CCC(n2cc(-c3cnc4ccccc4n3)cn2)CC1 ; "CH3I" ; CC(CCCO[Si](C)(C)C(C)(C)C)C(=O)N1CCC(n2cc(-c3cnc4ccccc4n3)cn2)CC1]

To a solution of 5-[tert-butyl(dimethyl)silyl]oxy-1-[4-(4-quinoxalin-2-ylpyrazol-1-yl)-1-piperidyl]pentan-1-one (850 mg, 1.72 mmol) in THF (30 mL) was added LiHMDS (3.44 mmol, 4 mL) at −78° C. After completion of addition, the reaction mixture was stirred at −78° C. for 0.5 h, at which time, CH₃I (733.42 mg, 5.16 mmol) was added. The reaction mixture was warmed to it slowly, quenched with aq. NH₄Cl, extracted with DCM (100 mL×3), dried over Na₂SO₄, concentrated and purified by silica gel chromatography (petroleum ether/EtOAc=1/1 to 1/2) to provide the title compound (320 mg, 36.6% yield). MS (ESI) m/z=507.5 [M+H]⁺.

Step 4: Synthesis of 5-hydroxy-2-methyl-1-(4-(4-(quinoxalin-2-yl)-1H-pyrazol-1-yl)piperidin-1-yl)pentan-1-one

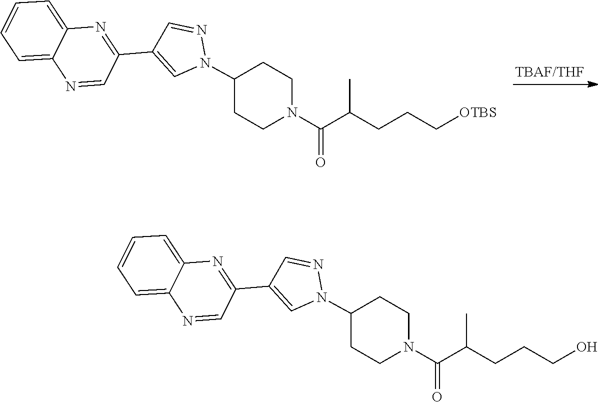 [SMILES: CC(CCCO[Si](C)(C)C(C)(C)C)C(=O)N1CCC(n2cc(-c3cnc4ccccc4n3)cn2)CC1 ; CC(CCCO)C(=O)N1CCC(n2cc(-c3cnc4ccccc4n3)cn2)CC1]

To a solution of 5-[tert-butyl(dimethyl)silyl]oxy-2-methyl-1-[4-(4-quinoxalin-2-ylpyrazol-1-yl)-1-piperidyl]pentan-1-one (400 mg, 787.80 μmol) in THF (10 mL) was added TBAF (1M, 4 mL) at rt. After the mixture was stirred at rt for 2 h, it was concentrated and purified by silica gel chromatography (DCM/MeOH=20/1) to provide the title compound (250 mg, 80.7% yield). MS (ESI) m/z=394.4 [M+H]⁺.

Step 5: Synthesis of 4-methyl-5-oxo-5-(4-(4-(quinoxalin-2-yl)-1H-pyrazol-1-yl)piperidin-1-yl)pentyl 4-methylbenzenesulfonate

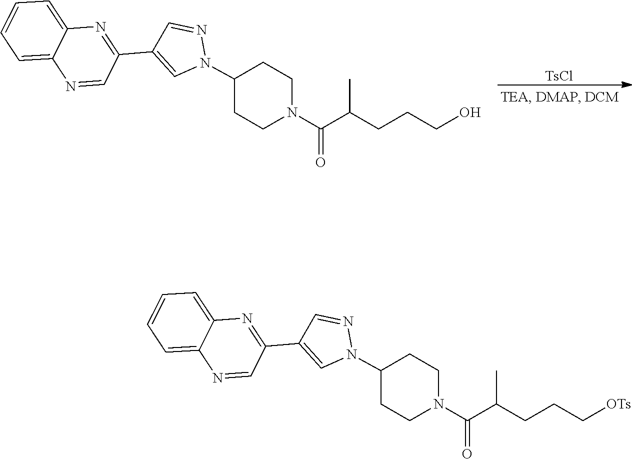 [SMILES: CC(CCCO)C(=O)N1CCC(n2cc(-c3cnc4ccccc4n3)cn2)CC1 ; "TsCl" ; Cc1ccc(S(=O)(=O)OCCCC(C)C(=O)N2CCC(n3cc(-c4cnc5ccccc5n4)cn3)CC2)cc1]

To a solution of 5-hydroxy-2-methyl-1-[4-(4-quinoxalin-2-ylpyrazol-1-yl)-1-piperidyl]pentan-1-one (250 mg, 635.35 μmol) in TEA (2 mL) and DCM (8 mL) were added TsCl (182.03 mg, 953.03 μmol) and DMAP (155.24 mg, 1.27 mmol) at rt. After competion of addition, the reaction mixture was stirred at rt for 3 h. The reaction was concentrated and purified by silica gel chromatography (DCM/MeOH=20/1) to provide the title compound (250 mg, 71.9% yield). MS (ESI) m/z=548.5 [M+H]⁺.

Step 6: Synthesis of 5-((4-methoxybenzyl)amino)-2-methyl-1-(4-(4-(quinoxalin-2-yl)-1H-pyrazol-1-yl)piperidin-1-yl)pentan-1-one

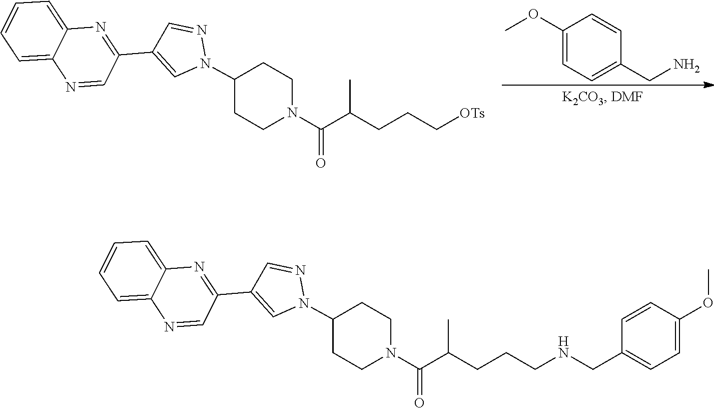

To a solution of [4-methyl-5-oxo-5-[4-(4-quinoxalin-2-ylpyrazol-1-yl)-1-piperidyl]pentyl] 4-methylbenzenesulfonate (250 mg, 456.48 μmol) and (4-methoxyphenyl)methanamine (62.62 mg, 456.48 μmol) in DMF (10 mL) were added NaI (136.84 mg, 912.96 μmol) and K₂CO₃ (188.98 mg, 1.37 mmol) at rt. After completion of addition, the reaction mixture was warmed to 50° C. and stirred for 16 h. The reaction was concentrated and purified by prep-HPLC to provide the title compound (190 mg, 66.4% yield). MS (ESI) m/z=513.6 [M+H]⁺.

Step 7: Synthesis of 2-(2,6-dioxopiperidin-3-yl)-5-((4-methoxybenzyl)(4-methyl-5-oxo-5-(4-(4-(quinoxalin-2-yl)-1H-pyrazol-1-yl)piperidin-1-yl)pentyl)amino)isoindoline-1,3-dione

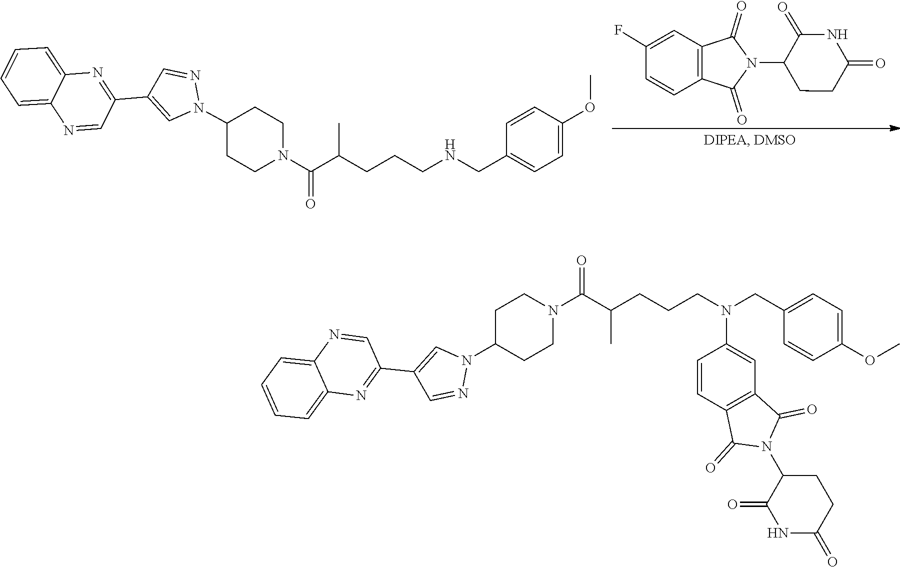 [SMILES: COc1ccc(CNCCCC(C)C(=O)N2CCC(n3cc(-c4cnc5ccccc5n4)cn3)CC2)cc1 ; O=C1CCC(N2C(=O)c3ccc(F)cc3C2=O)C(=O)N1 ; COc1ccc(CN(CCCC(C)C(=O)N2CCC(n3cc(-c4cnc5ccccc5n4)cn3)CC2)c2ccc3c(c2)C(=O)N(C2CCC(=O)NC2=O)C3=O)cc1]

To a solution of 5-[(4-methoxyphenyl)methylamino]-2-methyl-1-[4-(4-quinoxalin-2-ylpyrazol-1-yl)-1-piperidyl]pentan-1-one (190 mg, 303.19 μmol) and 2-(2,6-dioxo-3-piperidyl)-5-fluoro-isoindoline-1,3-dione (83.75 mg, 303.19 μmol) in DMSO (5 mL) was added DIPEA (391.84 mg, 3.03 mmol) at rt. After the mixture was warmed to 100° C. and stirred overnight, it was purified by prep-HPLC to provide the title compound (90 mg, 33.6% yield). MS (ESI) m/z=769.7 [M+H]⁺.

Step 8: Synthesis of 2-(2,6-dioxopiperidin-3-yl)-5-((4-methyl-5-oxo-5-(4-(4-(quinoxalin-2-yl)-1H-pyrazol-1-yl)piperidin-1-yl)pentyl)amino)isoindoline-1,3-dione

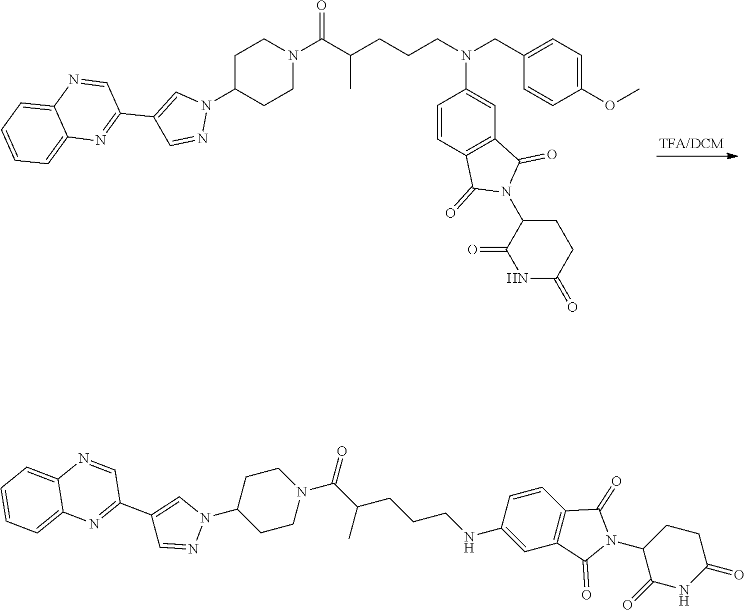 [SMILES: COc1ccc(CN(CCCC(C)C(=O)N2CCC(n3cc(-c4cnc5ccccc5n4)cn3)CC2)c2ccc3c(c2)C(=O)N(C2CCC(=O)NC2=O)C3=O)cc1 ; CC(CCCNc1ccc2c(c1)C(=O)N(C1CCC(=O)NC1=O)C2=O)C(=O)N1CCC(n2cc(-c3cnc4ccccc4n3)cn2)CC1]

To a solution of 2-(2,6-dioxo-3-piperidyl)-5-[(4-methoxyphenyl)methyl-[4-methyl-5-oxo-5-[4-(4-quinoxalin-2-ylpyrazol-1-yl)-1-piperidyl]pentyl]amino]isoindoline-1,3-dione (90 mg, 101.94 μmol, TF) in DCM (5 mL) was added TFA (1 mL) at rt. After the mixture was stirred at rt for 0.5 h, it was concentrated and purified by prep-TLC(DCM/MeOH=10/1) to provide the title compound (15 mg, 22.7% yield). MS (ESI) m/z=649.5 [M+H]⁺.

### Example 97. 5-((4,4-Dimethyl-5-oxo-5-(4-(4-(quinoxalin-2-yl)-1H-pyrazol-1-yl)piperidin-1-yl)pentyl)amino)-2-(2,6-dioxopiperidin-3-yl)isoindoline-1,3-dione (GS-139)

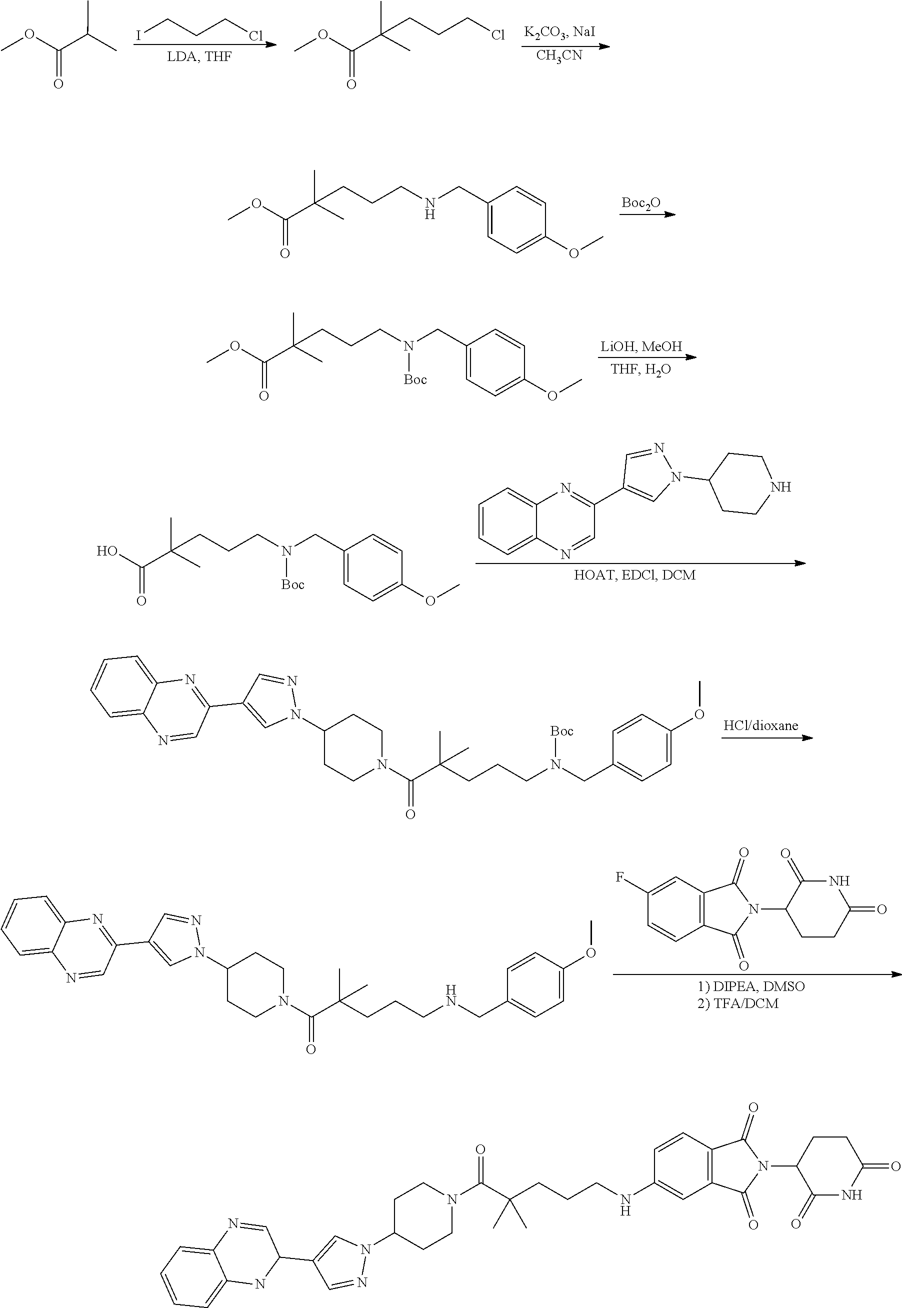

Step 1: Synthesis of methyl 5-chloro-2,2-dimethylpentanoate

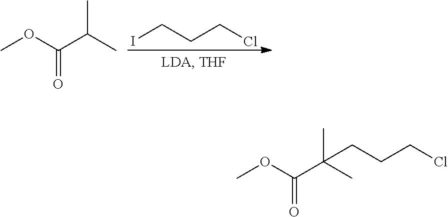

To a solution of methyl 2-methylpropanoate (3.0 g, 29.37 mmol) in THF (40 mL) was added LDA (44.06 mmol, 22 mL) at −78° C. After completion of addition, the reaction mixture was stirred at −78° C. for 0.5 h, at which time, 1-chloro-3-iodo-propane (6.61 g, 32.31 mmol) was added. The reaction mixture was warmed to rt slowly for 1 h, before it was quenched with HCl (1M, 40 ml), extracted with EtOAc (100 mL×3), dried over Na₂SO₄, concentrated and purified by silica gel chromatography (petroleum ether/EtOAc=50/1 to 30/1) to provide the title compound (5.0 g, 95.3% yield).

Step 2: Synthesis of methyl 5-((tert-butoxycarbonyl)(4-methoxybenzyl)amino)-2,2-dimethylpentanoate

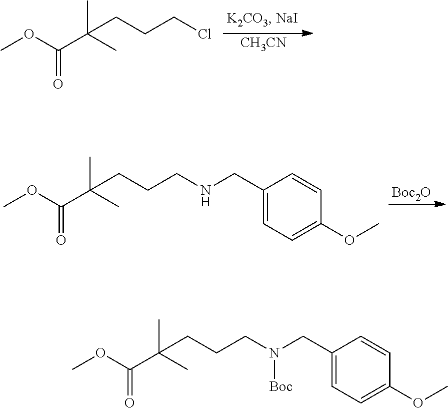

To a solution of methyl 5-chloro-2,2-dimethyl-pentanoate (3.56 g, 19.93 mmol) and (4-methoxyphenyl)methanamine (3.55 g, 25.90 mmol) in CH₃CN (50 mL) was added K₂CO₃ (8.25 g, 59.78 mmol) and NaI (2.99 g, 19.93 mmol) at rt. The reaction mixture was warmed to 80° C. and stirred for 16 h. After being cooled to rt, Boc₂O (6.52 g, 29.89 mmol) was added. The mixture was stirred at rt for 2 h, before it was concentrated and purified by silica gel chromatography (petroleum ether/EtOAc=20/1 to 10/1) to provide the title compound (6.0 g, 79.4% yield). MS (ESI) m/z=380.3 [M+H]⁺.

Step 3: Synthesis of 5-((tert-butoxycarbonyl)(4-methoxybenzyl)amino)-2,2-dimethylpentanoic acid

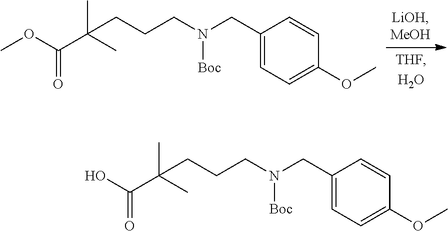

To a solution of methyl 5-[tert-butoxycarbonyl-[(4-methoxyphenyl)methyl]amino]-2,2-dimethyl-pentanoate (6.0 g, 15.81 mmol) in MeOH (30 mL) and THF (30 mL) were added LiOH (3.32 g, 79.05 mmol) and water (30 mL). After the mixture was stirred at rt for 8 h, HCl (1 M) was added to adjust pH to -3. The reaction was extracted with DCM (100 mL×3), dried over Na₂SO₄, concentrated to provide the title compound (3.5 g, 60.6% yield). MS (ESI) m/z=310.1 [M−56+H]⁺.

Step 4: Synthesis of tert-butyl (4,4-dimethyl-5-oxo-5-(4-(4-(quinoxalin-2-yl)-1H-pyrazol-1-yl)piperidin-1-yl)pentyl)(4-methoxybenzyl)carbamate

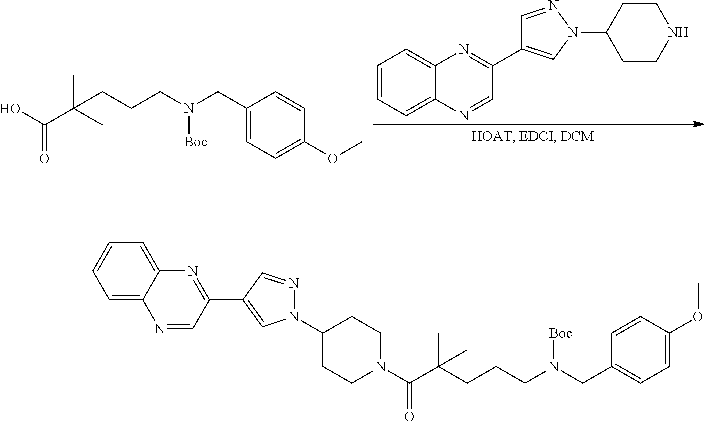

To a solution of 5-[tert-butoxycarbonyl-[(4-methoxyphenyl)methyl]amino]-2,2-dimethyl-pentanoic acid (365 mg, 998.73 μmol) and 2-[1-(4-piperidyl)pyrazol-4-yl]quinoxaline (278.99 mg, 998.73 μmol) in THF (20 mL) were added HOAt (202.24 mg, 1.50 mmol), EDCI (287.64 mg, 1.50 mmol) and TEA (303.19 mg, 3.00 mmol) at rt. After completion of addition, the reaction mixture was stirred at rt for 16 h. The reaction was concentrated and purified by silica gel chromatography (DCM/MeOH=20/1) to provide the title compound (400 mg, 63.9% yield) as a yellow solid. MS (ESI) m/z=627.7 [M+H]⁺.

Step 5: Synthesis of 5-((4-methoxybenzyl)amino)-2,2-dimethyl-1-(4-(4-(quinoxalin-2-yl)-1H-pyrazol-1-yl)piperidin-1-yl)pentan-1-one

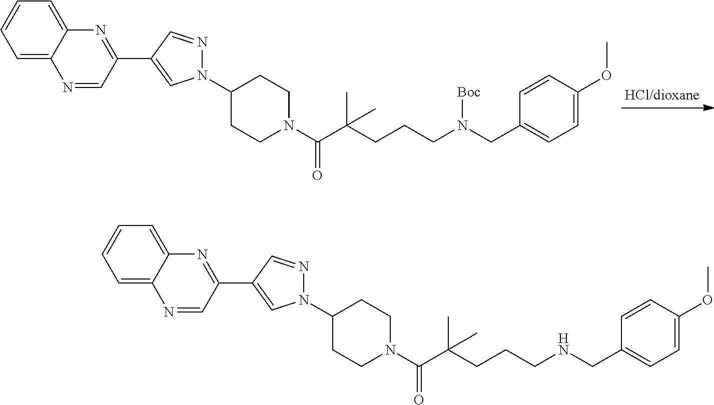 [SMILES: COc1ccc(CN(CCCC(C)(C)C(=O)N2CCC(n3cc(-c4cnc5ccccc5n4)cn3)CC2)C(=O)OC(C)(C)C)cc1 ; COc1ccc(CNCCCC(C)(C)C(=O)N2CCC(n3cc(-c4cnc5ccccc5n4)cn3)CC2)cc1]

To a solution of tert-butyl N-[4,4-dimethyl-5-oxo-5-[4-(4-quinoxalin-2-ylpyrazol-1-yl)-1-piperidyl]pentyl]-N-[(4-methoxyphenyl)methyl]carbamate (400 mg, 638.18 μmol) in DCM (10 mL) was added HCl/dioxane (1 M, 4 mL) at rt. After the mixture was stirred at rt for 2 h, it was concentrated and purified by prep-HPLC to provide the title compound (340 mg, 83.2% yield) as yellow solid. MS (ESI) m/z=527.5 [M+H]⁺.

Step 6: Synthesis of 5-((4,4-dimethyl-5-oxo-5-(4-(4-(quinoxalin-2-yl)-1H-pyrazol-1-yl)piperidin-1-yl)pentyl)amino)-2-(2,6-dioxopiperidin-3-yl)isoindoline-1,3-dione

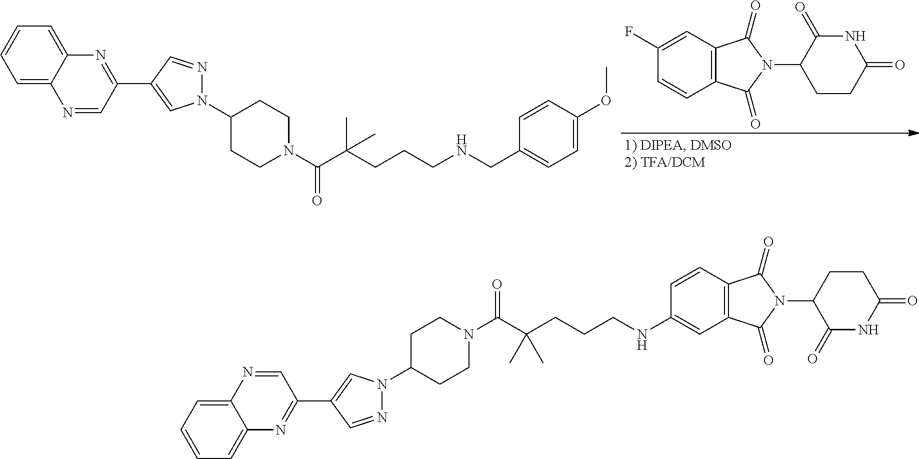

GS-139 was synthesized following the similar procedure for preparing GS-138 (38 mg, yield: 10.8%). MS (ESI) m/z=663.5 [M+H]⁺.

### Example 98. 3-(1-Oxo-5-((3-(3-(4-(quinoxalin-2-yl)-1H-pyrazol-1-yl)cyclobutyl)propyl)amino)isoindolin-2-yl)piperidine-2,6-dione (GS-140)

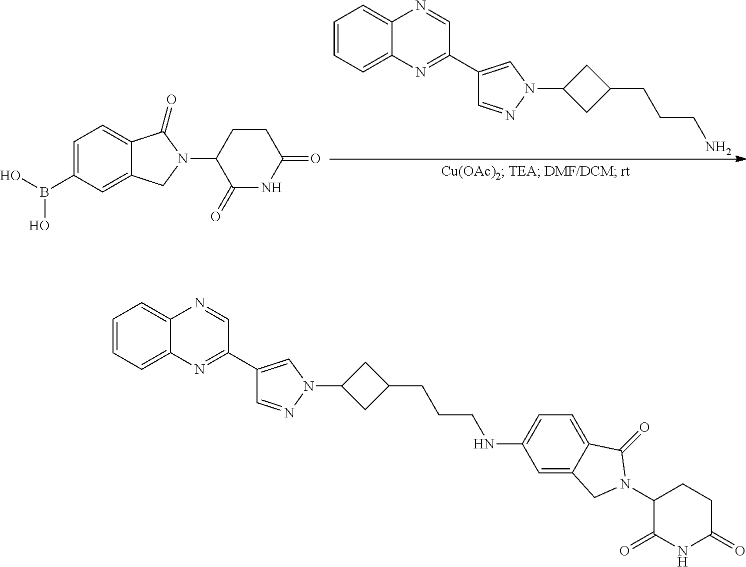 [SMILES: O=C1CCC(N2Cc3cc(B(O)O)ccc3C2=O)C(=O)N1 ; NCCCC1CC(n2cc(-c3cnc4ccccc4n3)cn2)C1 ; O=C1CCC(N2Cc3cc(NCCCC4CC(n5cc(-c6cnc7ccccc7n6)cn5)C4)ccc3C2=O)C(=O)N1]

GS-140 was synthesized following the similar procedure for preparing GS-126 (12.2 mg, yield: 32.2%). MS (ESI) m/z=550.4 [M+H]⁺.

### Example 99. 2-(2,6-Dioxopiperidin-3-yl)-5-((3-(1-(4-(4-(quinoxalin-2-yl)-1H-pyrazol-1-yl)piperidine-1-carbonyl)cyclopropyl)propyl)amino)isondoline-1,3-dione (GS-141)

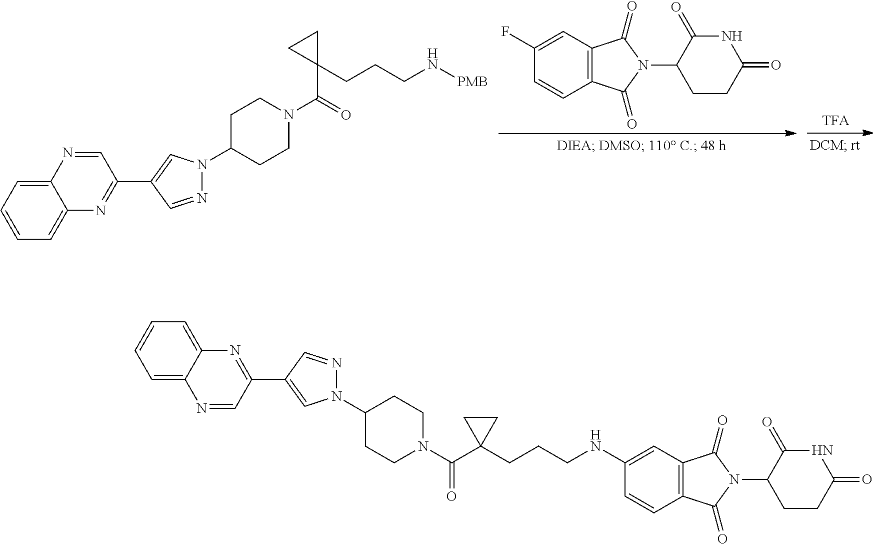

GS-141 was synthesized following the similar procedure for preparing GS-139 (8.2 mg, yield: 10%). MS (ESI) m/z=661.5 [M+H]⁺.

### Example 100. 3-(1-Oxo-4-((3-(4-(4-(quinoxalin-2-yl)-1H-pyrazol-1-yl)butyl)cyclohexyl)amino)isoindolin-2-yl)piperidine-2,6-dione (GS-142)

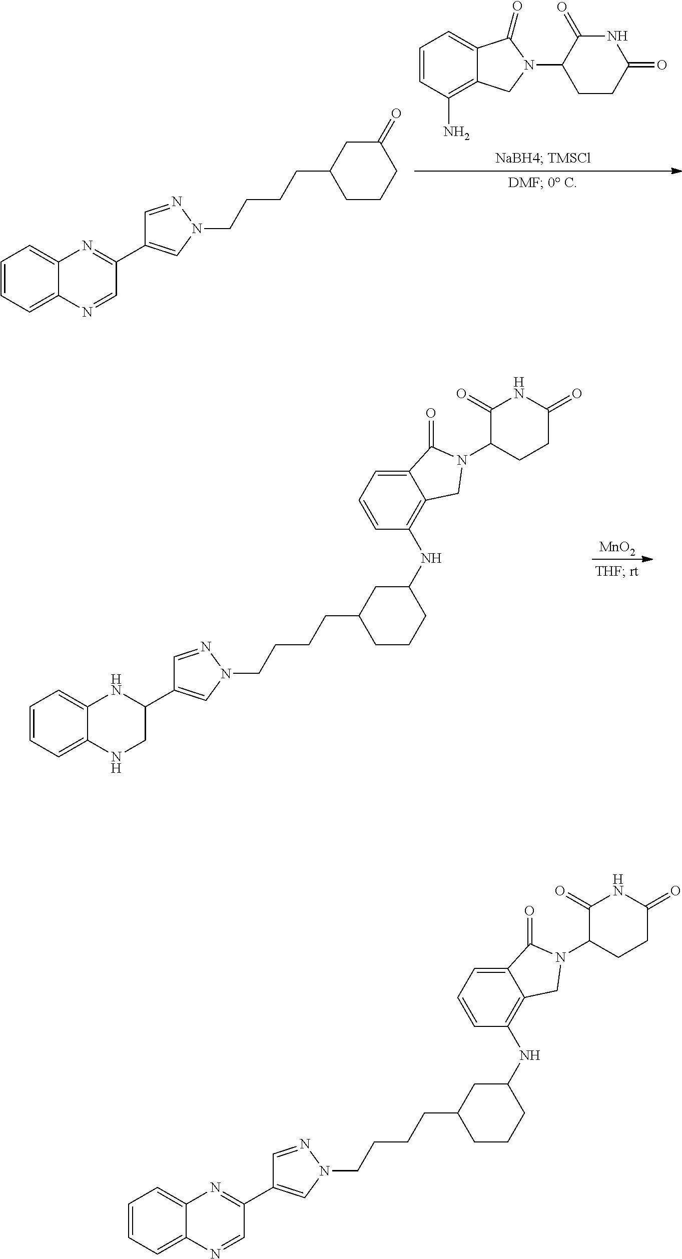

GS-142 was synthesized following the similar procedure for preparing GS-154 (35 mg, yield: 68%). MS (ESI) m/z=592.3 [M+H]⁺.

### Example 101. 5-((5-((2R,6S)-2,6-dimethyl-4-(4-(quinoxalin-2-yl)-1H-pyrazol-1-yl)piperidin-1-yl)-5-oxopentyl)amino)-2-(2,6-dioxopiperidin-3-yl)isoindoline-1,3-dione (GS-143)

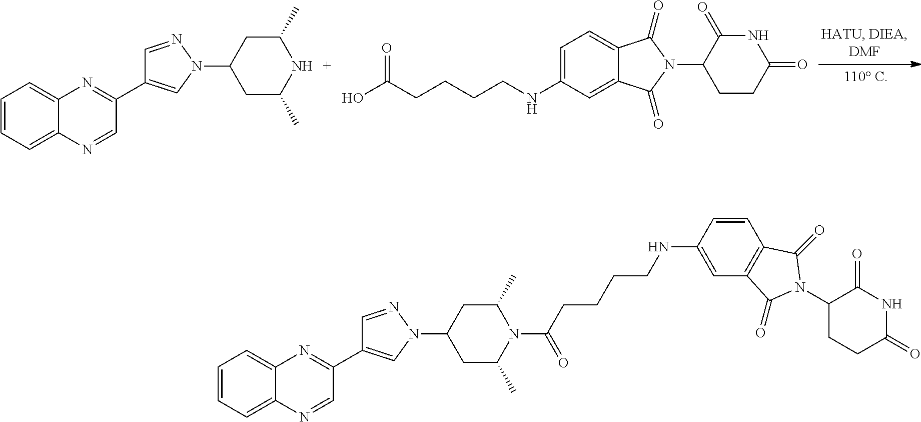 [SMILES: C[C@@H]1CC(n2cc(-c3cnc4ccccc4n3)cn2)C[C@H](C)N1 ; O=C(O)CCCCNc1ccc2c(c1)C(=O)N(C1CCC(=O)NC1=O)C2=O ; C[C@@H]1CC(n2cc(-c3cnc4ccccc4n3)cn2)C[C@H](C)N1C(=O)CCCCNc1ccc2c(c1)C(=O)N(C1CCC(=O)NC1=O)C2=O]

GS-143 was synthesized following the similar procedure for preparing GS-005 (12 mg, yield: 29.4%). MS (ESI) m/z=663.5 [M+H]⁺.

### Example 102. 2-(2,6-Dioxopiperidin-3-yl)-5-(((3-(4-(4-(quinoxalin-2-yl)-1H-pyrazol-1-yl)piperidine-1-carbonyl)bicyclo[1.1.1]pentan-1-yl)methyl)amino)isoindoline-1,3-dione (GS-144)

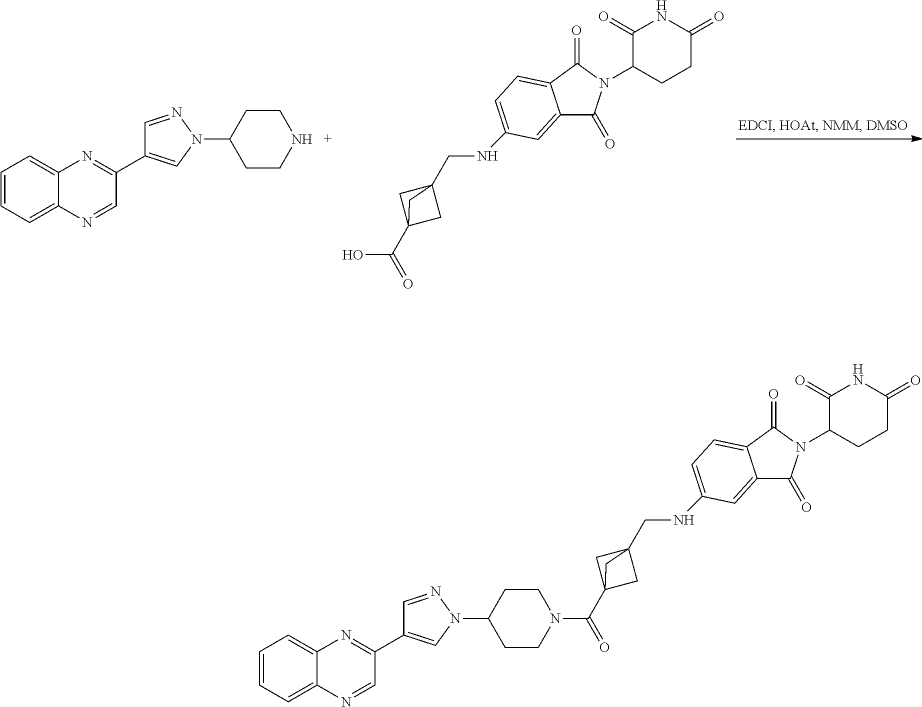 [SMILES: c1ccc2nc(-c3cnn(C4CCNCC4)c3)cnc2c1 ; O=C1CCC(N2C(=O)c3ccc(NCC45CC(C(=O)O)(C4)C5)cc3C2=O)C(=O)N1 ; O=C1CCC(N2C(=O)c3ccc(NCC45CC(C(=O)N6CCC(n7cc(-c8cnc9ccccc9n8)cn7)CC6)(C4)C5)cc3C2=O)C(=O)N1]

GS-144 was synthesized following the similar procedure for preparing GS-001 (5.3 mg, yield: 12%). MS (ESI) m/z=659.4 [M+H]⁺.

### Example 103. 5-((4,4-Difluoro-5-oxo-5-(4-(4-(quinoxalin-2-yl)-1H-pyrazol-1-yl)piperidin-1-yl)pentyl)amino)-2-(2,6-dioxopiperidin-3-yl)isoindoline-1,3-dione (GS-145)

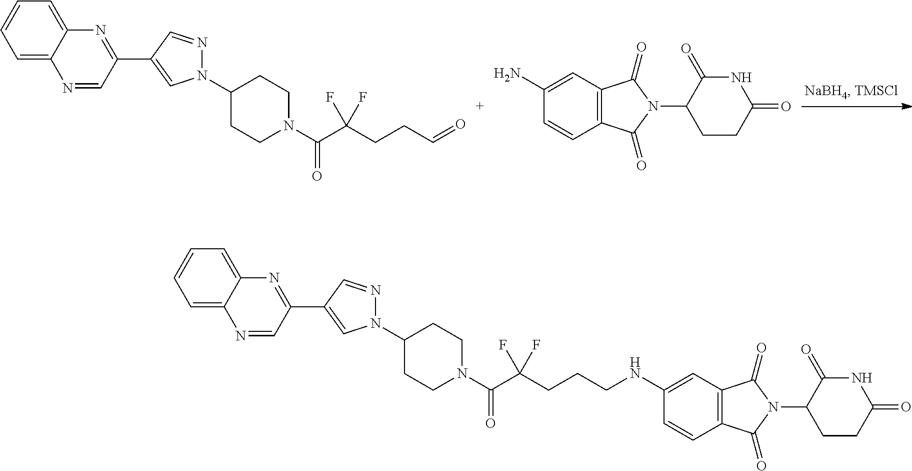 [SMILES: O=CCCC(F)(F)C(=O)N1CCC(n2cc(-c3cnc4ccccc4n3)cn2)CC1 ; Nc1ccc2c(c1)C(=O)N(C1CCC(=O)NC1=O)C2=O ; O=C1CCC(N2C(=O)c3ccc(NCCCC(F)(F)C(=O)N4CCC(n5cc(-c6cnc7ccccc7n6)cn5)CC4)cc3C2=O)C(=O)N1]

GS-145 was synthesized following the similar procedure for preparing GS-162 (2.8 mg, yield: 11.5%). MS (ESI) m/z=671.5 [M+H]⁺.

### Example 104. 5-((6-(3,5-Dimethyl-4-(pyrazin-2-yl)-1H-pyrazol-1-yl)hexyl)amino)-2-(2,6-dioxopiperidin-3-yl)isoindoline-1,3-dione (GS-146)

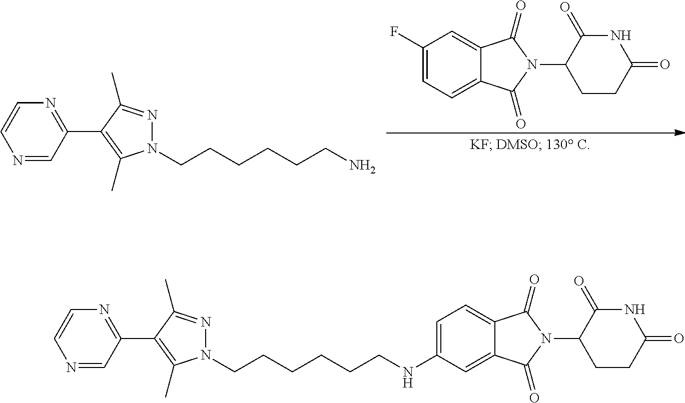 [SMILES: Cc1nn(CCCCCCN)c(C)c1-c1cnccn1 ; O=C1CCC(N2C(=O)c3ccc(F)cc3C2=O)C(=O)N1 ; Cc1nn(CCCCCCNc2ccc3c(c2)C(=O)N(C2CCC(=O)NC2=O)C3=O)c(C)c1-c1cnccn1]

GS-146 was synthesized following the similar procedure for preparing GS-150 (0.8 mg, yield: 4%). MS (ESI) m/z=530.4 [M+H]⁺.

### Example 105. 2-(2,6-Dioxopiperidin-3-yl)-5-((5-oxo-5-(4-phenylpiperidin-1-yl)pentyl)amino)isoindoline-1,3-dione (GS-147)

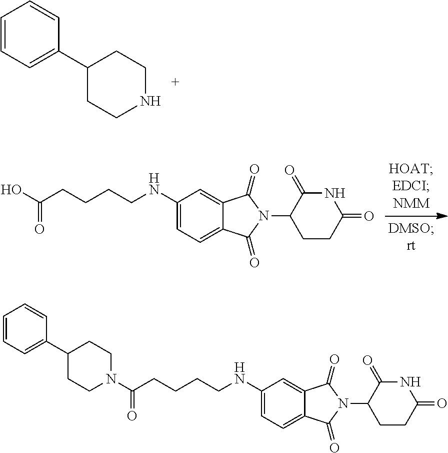 [SMILES: c1ccc(C2CCNCC2)cc1 ; O=C(O)CCCCNc1ccc2c(c1)C(=O)N(C1CCC(=O)NC1=O)C2=O ; O=C1CCC(N2C(=O)c3ccc(NCCCCC(=O)N4CCC(c5ccccc5)CC4)cc3C2=O)C(=O)N1]

GS-147 was synthesized following the similar procedure for preparing GS-001 (22 mg, yield: 42.6%). MS (ESI) m/z=517.4 [M+H]⁺.

### Example 106. 2-(2,6-Dioxopiperidin-3-yl)-5-((4-oxo-4-(4-phenylpiperidin-1-yl)butyl)amino)isoindoline-1,3-dione (GS-148)

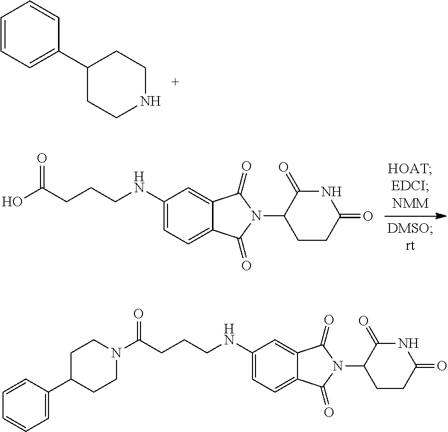 [SMILES: c1ccc(C2CCNCC2)cc1 ; O=C(O)CCCNc1ccc2c(c1)C(=O)N(C1CCC(=O)NC1=O)C2=O ; O=C1CCC(N2C(=O)c3ccc(NCCCC(=O)N4CCC(c5ccccc5)CC4)cc3C2=O)C(=O)N1]

GS-148 was synthesized following the similar procedure for preparing GS-001 (18 mg, yield: 35.9%). MS (ESI) m/z=503.3 [M+H]⁺.

### Example 107. N-(2-((2-(2,6-dioxopiperidin-3-yl)-1,3-dioxoisoindolin-5-yl)amino)ethyl)-4-(4-(quinoxalin-2-yl)-1H-pyrazol-1-yl)piperidine-1-carboxamide (GS-149)

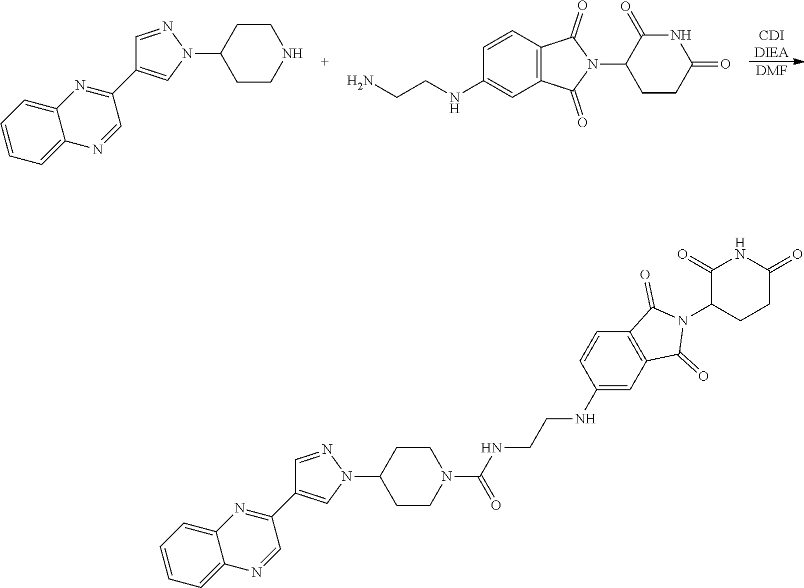

A mixture of CDI (28 mg, 0.17 mmol) and DIEA (41 mg, 0.32 mmol) in anhydrous DMF (3.0 mL) was stirred at rt for 10 min, before 5-((2-aminoethyl)amino)-2-(2,6-dioxopiperidin-3-yl)isoindoline-1,3-dione (50 mg, 0.16 mmol) was added. After the mixture was stirred at rt for 40 min, 2-(1-(piperidin-4-yl)-1H-pyrazol-4-yl)quinoxaline (45 mg, 0.16 mmol) was added. The resulting mixture was stirred at 60° C. for 16 h, before the mixture was purified by reverse phase column to provide the title compound (50 mg, 50.9% yield) as a brown solid. MS (ESI) m/z=622.4 [M+H]⁺.

### Example 108. 5-((6-(3,5-Dimethyl-4-(pyrimidin-2-yl)-1H-pyrazol-1-yl)hexyl)amino)-2-(2,6-dioxopiperidin-3-yl)isoindoline-1,3-dione (GS-150)

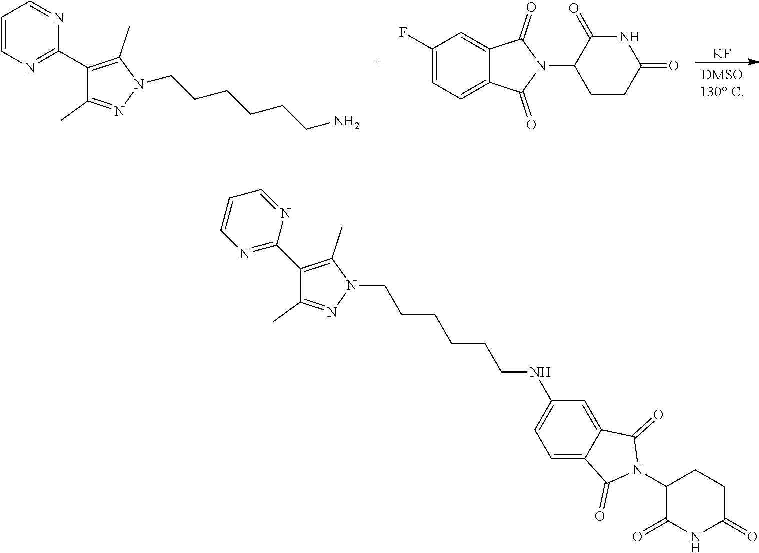

A mixture of 6-(3,5-dimethyl-4-pyrimidin-2-yl-pyrazol-1-yl)hexan-1-amine (40 mg, 0.1 mmol), 2-(2,6-dioxo-3-piperidyl)-5-fluoro-isoindoline-1,3-dione (36 mg, 0.13 mmol) and KF (151 mg, 2.61 mmol) in DMSO (1.5 mL) was stirred at 130° C. for 1 h under microwave irradiation. The mixture was purified by reverse-phase chromatography to provide the title compound (4.7 mg, 6.8% yield) as a white solid. MS (ESI) m/z=530.5 [M+H]⁺.

### Example 109. 5-((6-(3,5-Dimethyl-4-(quinazolin-2-yl)-1H-pyrazol-1-yl)hexyl)amino)-2-(2,6-dioxopiperidin-3-yl)isoindoline-1,3-dione (GS-151)

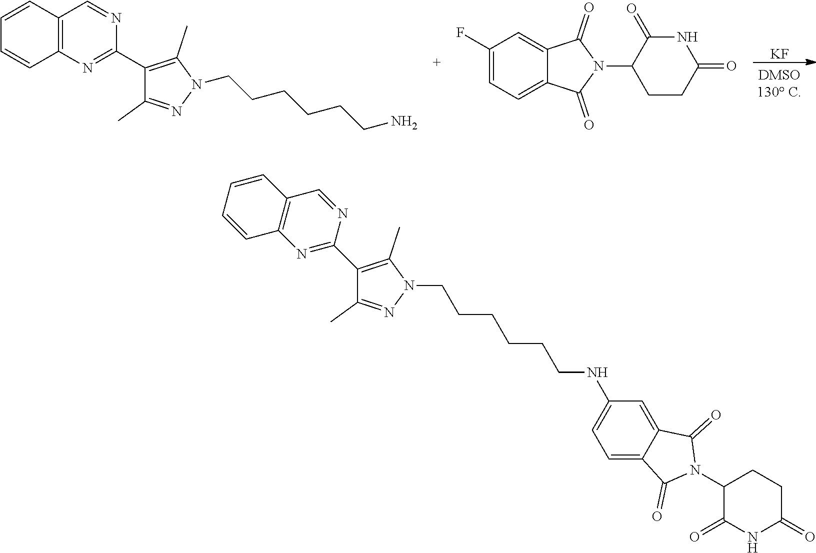

GS-151 was synthesized following the similar procedure for preparing GS-150 (3.6 mg, yield: 4.6%). MS (ESI) m/z=580.4 [M+H]⁺.

### Example 110. 2-(2,6-Dioxopiperidin-3-yl)-5-((3-(4-(4-(quinoxalin-2-yl)-1H-pyrazol-1-yl)piperidin-1-yl)phenethyl)amino)isoindoline-1,3-dione (GS-152)

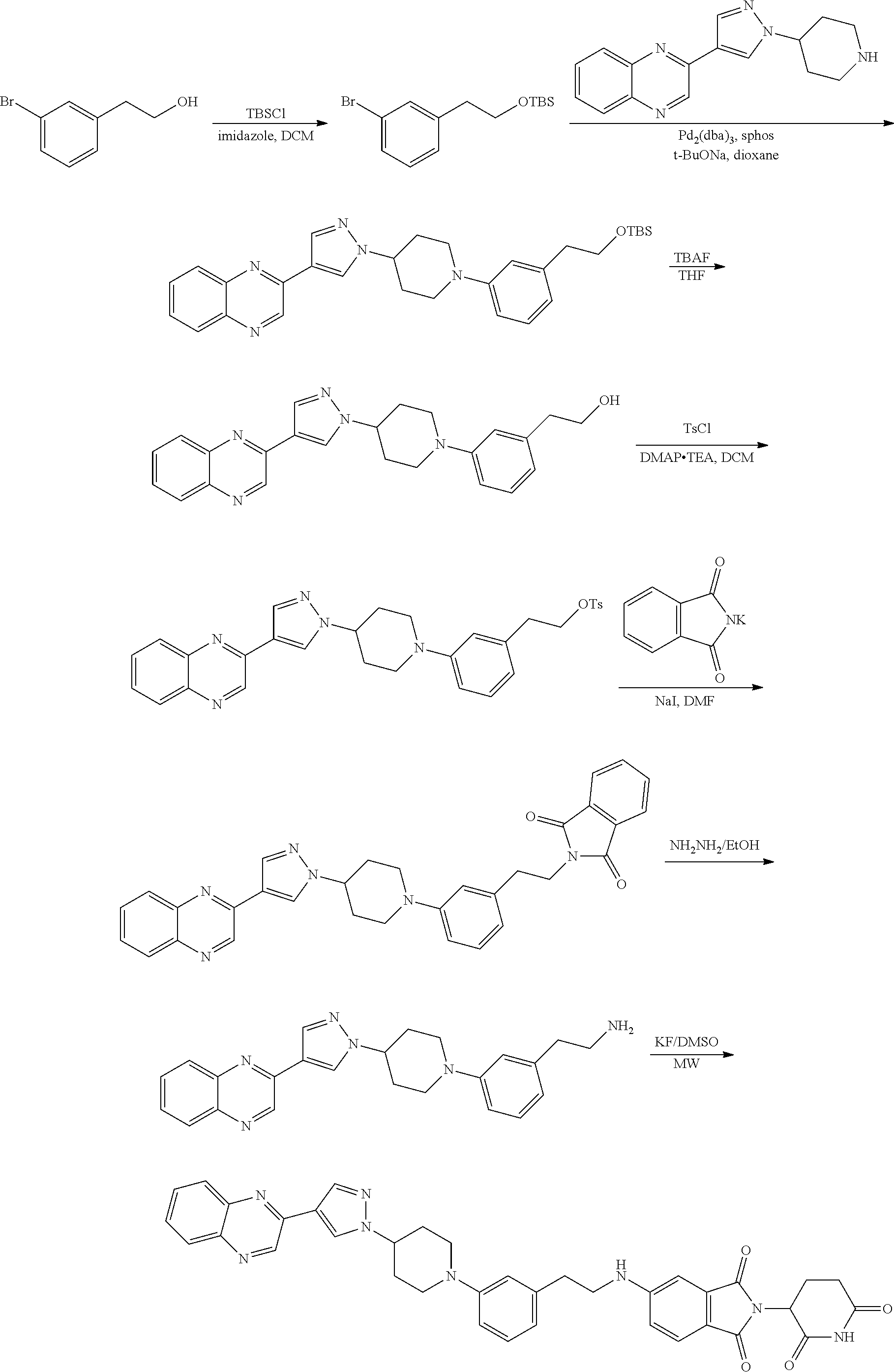

Step 1: Synthesis of (3-bromophenethoxy)(tert-butyl)dimethylsilane

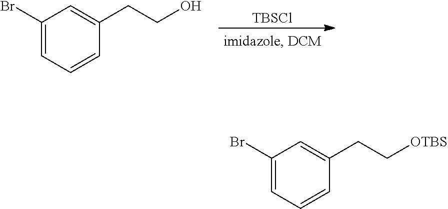 [SMILES: OCCc1cccc(Br)c1 ; "TBSCl" ; CC(C)(C)[Si](C)(C)OCCc1cccc(Br)c1]

To a solution of 2-(3-bromophenyl)ethanol (5.0 g, 24.87 mmol) in DCM (50 mL) were added imidazole (3.39 g, 49.74 mmol) and TBSCl (5.62 g, 37.30 mmol) at rt. After the reaction mixture was stirred at rt for 2 h, it was concentrated and purified by silica gel chromatography (PE) to provide the title compound (7.8 g, 99.5% yield) as a colorless oil.

Step 2: Synthesis of 2-(1-(1-(3-(2-((tert-butyldimethylsilyl)oxy)ethyl)phenyl)piperidin-4-yl)-1H-pyrazol-4-yl)quinoxaline

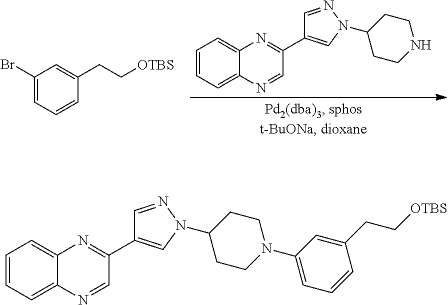 [SMILES: CC(C)(C)[Si](C)(C)OCCc1cccc(Br)c1 ; c1ccc2nc(-c3cnn(C4CCNCC4)c3)cnc2c1 ; CC(C)(C)[Si](C)(C)OCCc1cccc(N2CCC(n3cc(-c4cnc5ccccc5n4)cn3)CC2)c1]

To a solution of 2-(3-bromophenyl)ethoxy-tert-butyl-dimethyl-silane (628 mg, 1.99 mmol) and 2-[1-(4-piperidyl)pyrazol-4-yl]quinoxaline (556.34 mg, 1.99 mmol) in dioxane (20 mL) was added Pd₂(dba)₃ (364.47 mg, 398.33 μmol), Sphos (245.57 mg, 597.49 μmol) and t-BuONa (573.59 mg, 5.97 mmol) at rt under N₂. The reaction mixture was stirred at 100° C. and monitored by LCMS. After the starting materials were consumed, the reaction mixture was cooled to rt and concentrated. The residue was purified by silica gel chromatography (DCM/MeOH=20/1) to provide the title compound (1.0 g, 97.7% yield) as a yellow solid. MS (ESI) m/z=514.6 [M+H]⁺.

Step 3: Synthesis of 2-(3-(4-(4-(quinoxalin-2-yl)-1H-pyrazol-1-yl)piperidin-1-yl)phenyl)ethan-1-ol

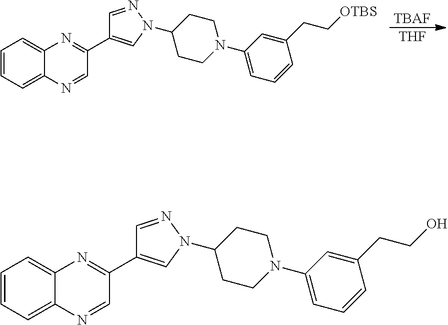

To a solution of tert-butyl-dimethyl-[2-[3-[4-(4-quinoxalin-2-ylpyrazol-1-yl)-1-piperidyl]phenyl]ethoxy]silane (1.1 g, 2.14 mmol) in THF (10 mL) was added TBAF (1M, 6 mL). After the reaction mixture was stirred at rt for 4 h, it was concentrated and purified by silica gel chromatography (DCM/MeOH=20/1) to provide the title compound (800 mg, 93.5% yield). MS (ESI) m/z=400.3 [M+H]⁺.

Step 4: Synthesis of 3-(4-(4-(quinoxalin-2-yl)-1H-pyrazol-1-yl)piperidin-1-yl)phenethyl 4-methylbenzenesulfonate

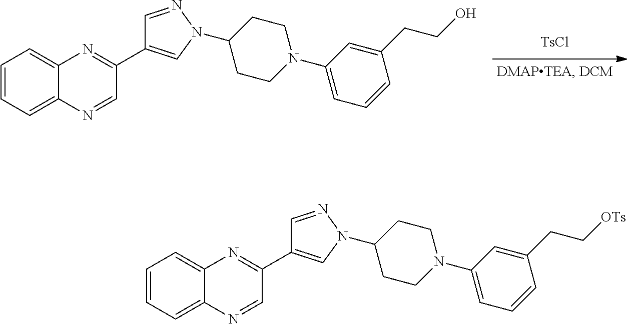 [SMILES: OCCc1cccc(N2CCC(n3cc(-c4cnc5ccccc5n4)cn3)CC2)c1 ; "TsCl" ; Cc1ccc(S(=O)(=O)OCCc2cccc(N3CCC(n4cc(-c5cnc6ccccc6n5)cn4)CC3)c2)cc1]

To a solution of 2-[3-[4-(4-quinoxalin-2-ylpyrazol-1-yl)-1-piperidyl]phenyl]ethanol (800 mg, 2.00 mmol) in TEA (5 mL) and DCM (15 mL) was added DMAP (24.47 mg, 200.26 μmol) and TsCl (458.99 mg, 2.40 mmol) at rt. After the reaction mixture was stirred at rt for 3 h, it was concentrated and purified by silica gel chromatography (DCM/MeOH=20/1) to provide the title compound (1.0 g, 90.2% yield) as a yellow solid. MS (ESI) m/z=554.5 [M+H]⁺.

Step 5: Synthesis of 2-(3-(4-(4-(quinoxalin-2-yl)-1H-pyrazol-1-yl)piperidin-1-yl)phenethyl)isoindoline-1,3-dione

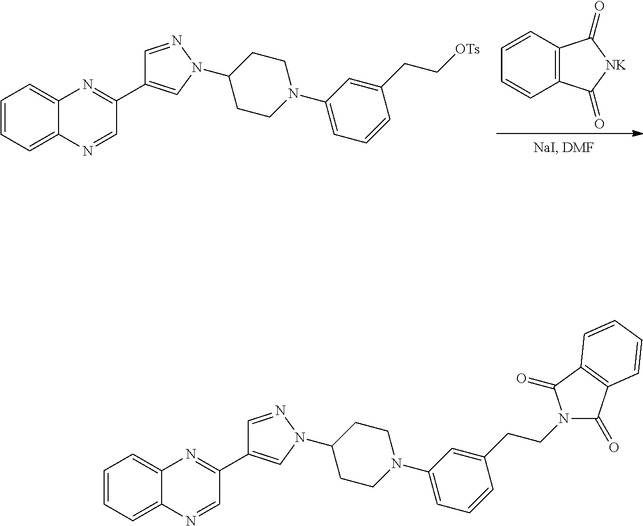

To a solution of 2-[3-[4-(4-quinoxalin-2-ylpyrazol-1-yl)-1-piperidyl]phenyl]ethyl 4-methylbenzenesulfonate (553 mg, 998.78 μmol) and (1,3-dioxoisoindolin-2-yl)potassium (369.99 mg, 2.00 mmol) in DMF (10 mL) was added NaI (299.42 mg, 2.00 mmol) at rt. After the reaction mixture was stirred at 45° C. for 16 h, it was cooled to rt and concentrated. The resulting residue was purified by silica gel chromatography (DCM/MeOH=20/1) to provide the title compound (500 mg, 94.7% yield) as a yellow solid. MS (ESI) m/z=529.5 [M+H]⁺.

Step 6: Synthesis of 2-(3-(4-(4-(quinoxalin-2-yl)-1H-pyrazol-1-yl)piperidin-1-yl)phenyl)ethan-1-amine

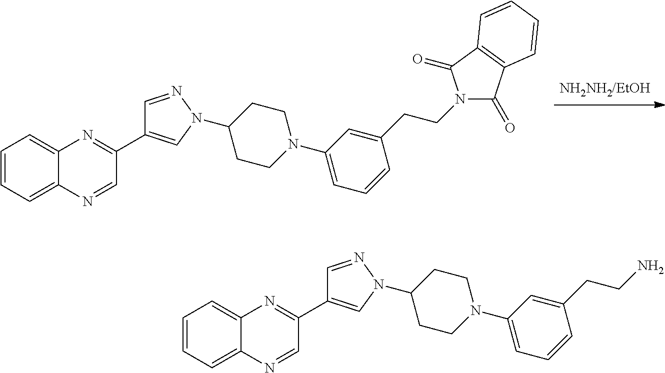 [SMILES: O=C1c2ccccc2C(=O)N1CCc1cccc(N2CCC(n3cc(-c4cnc5ccccc5n4)cn3)CC2)c1 ; NCCc1cccc(N2CCC(n3cc(-c4cnc5ccccc5n4)cn3)CC2)c1]

To a solution of 2-[2-[3-[4-(4-quinoxalin-2-ylpyrazol-1-yl)-1-piperidyl]phenyl]ethyl]isoindoline-1,3-dione (500 mg, 945.89 μmol) in EtOH (10 mL) was added NH₂NH₂ (2 mL) at rt. After the reaction mixture sitrred at 50° C. for 2 h, it was purified by prep-HPLC to provide the title compound (400 mg, 82.5% yield). MS (ESI) m/z=399.3 [M+H]⁺.

Step 7: Synthesis of 2-(2,6-dioxopiperidin-3-yl)-5-((3-(4-(4-(quinoxalin-2-yl)-1H-pyrazol-1-yl)piperidin-1-yl)phenethyl)amino)isoindoline-1,3-dione

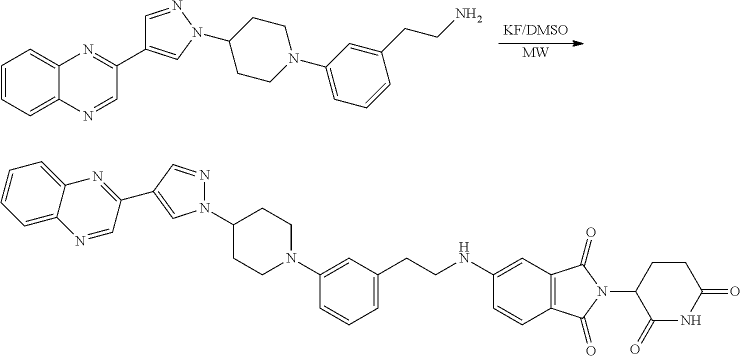

To a solution of 2-[3-[4-(4-quinoxalin-2-ylpyrazol-1-yl)-1-piperidyl]phenyl]ethanamine (100 mg, 195.11 μmol) and 2-(2,6-dioxo-3-piperidyl)-5-fluoro-isoindoline-1,3-dione (107.79 mg, 390.22 μmol) in DMSO (5 mL) was added KF (113.36 mg, 1.95 mmol) at rt. After the reaction mixture was irradiated in microwave at 135° C. for 1 h, it was purified by prep-HPLC, followed by prep-TLC (DCM/MeOH=15/1) to provide the title compound (23 mg, 18% yield) as a yellow solid. MS (ESI) m/z=655.5 [M+H]⁺.

### Example 111. 2-(2,6-Dioxopiperidin-3-yl)-5-((5-oxo-5-(7-(4-(quinoxalin-2-yl)-1H-pyrazol-1-yl)-4-azaspiro[2.5]octan-4-yl)pentyl)amino)isoindoline-1,3-dione (GS-153)

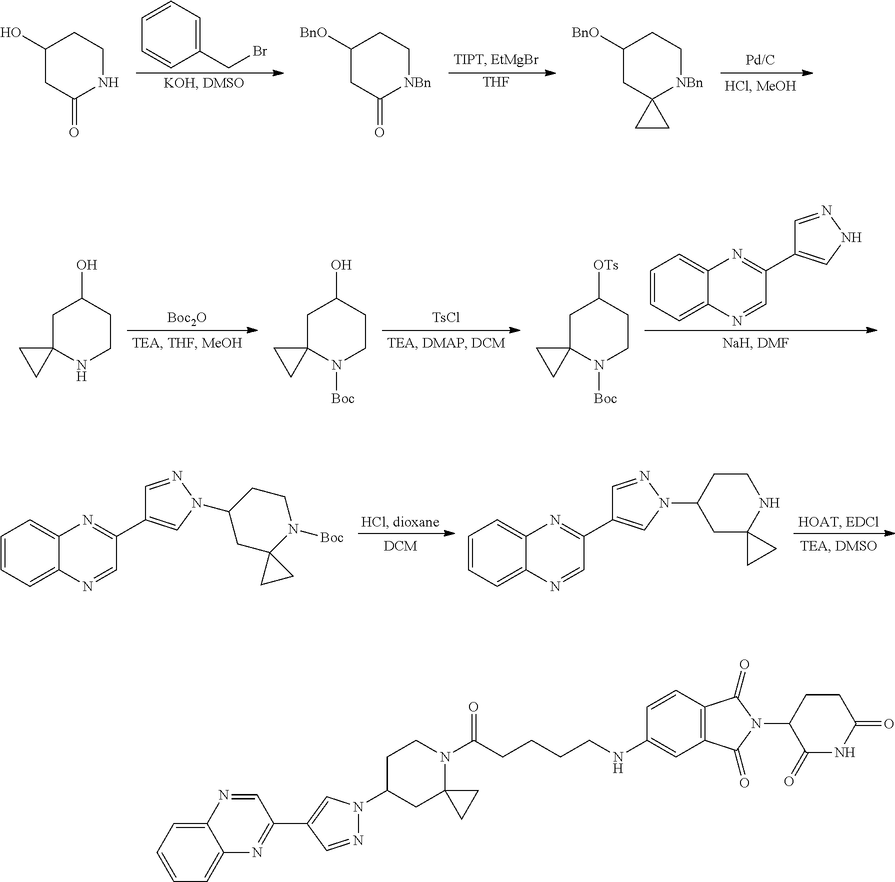

Step 1: Synthesis of 1-benzyl-4-(benzyloxy)piperidin-2-one

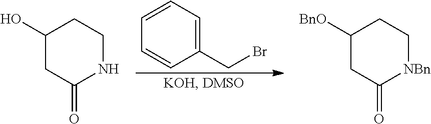

To a solution of 4-hydroxypiperidin-2-one (5.0 g, 43.43 mmol) in DMSO (100 mL) was added potassium hydroxide (19.49 g, 347.43 mmol) at rt. After the completion of addition, the reaction mixture was stirred at rt for 0.5 h, at which time, bromomethylbenzene (22.28 g, 130.29 mmol) was added. After the mixture was stirred at rt for another 2 h, it was quenched with HCl (1M, 20 mL), extracted with EtOAc (50 mL×3), dried over Na₂SO₄, concentrated and purified silica gel chromatography (petroleum ether/EtOAc=10/1 to 1/1) to provide the title compound (6.0 g, 46.8% yield) as a colorless oil which was used in the next step without further characterization.

Step 2: Synthesis of 4-benzyl-7-(benzyloxy)-4-azaspiro[2.5]octane

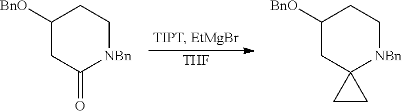 [SMILES: O=C1CC(OCc2ccccc2)CCN1Cc1ccccc1 ; c1ccc(COC2CCN(Cc3ccccc3)C3(CC3)C2)cc1 ; "THF"]

To a solution of EtMgBr (81.25 mmol, 40 mL) in THF (300 mL) was added TIPT (17.31 g, 60.94 mmol) at −78° C. After completion of addition, the reaction mixture was stirred at −78° C. for 0.5 h, at which time, 1-benzyl-4-benzyloxy-piperidin-2-one (6.0 g, 20.31 mmol) was added. The mixture was stirred at rt for 1 h, before it was warmed to 70° C. and stirred for another 10 h. The reaction was quenched with aq. NH₄Cl (100 mL), extracted with EtOAc (200 mL×3), dried over Na₂SO₄, concentrated and purified by silica gel chromatography (petroleum ether/EtOAc=15/1 to 10/1) to provide the title compound (2.45 g, 39.2% yield) as a colorless oil. MS (ESI) m/z=308.4 [M+H]⁺.

Step 3: Synthesis of 4-azaspiro[2.5]octan-7-ol

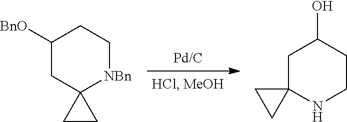 [SMILES: c1ccc(COC2CCN(Cc3ccccc3)C3(CC3)C2)cc1 ; OC1CCNC2(CC2)C1]

To a solution of 4-benzyl-7-benzyloxy-4-azaspiro[2.5]octane (2.45 g, 7.97 mmol) in MeOH (100 mL) was added HCl (1 mL) and Pd/C (1.0 g, 975.84 μmol) at rt under H₂. After the reaction mixture was stirred at rt for 2 days, it was filtrated and concentrated to provide the title compound (1.3 g, 99.7% yield) which was used directly in the next step.

Step 4: Synthesis of tert-butyl 7-hydroxy-4-azaspiro[2.5]octane-4-carboxylate

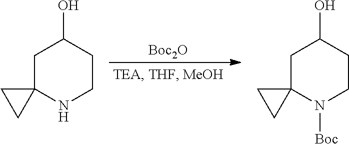 [SMILES: "Boc2O" ; OC1CCNC2(CC2)C1 ; CC(C)(C)OC(=O)N1CCC(O)CC12CC2]

To a solution of 4-azaspiro[2.5]octan-7-ol (1.3 g, 7.94 mmol) in MeOH (20 mL) and THF (50 mL) were added TEA (4.82 g, 47.66 mmol) and Boc₂O (2.60 g, 11.92 mmol) at rt. After the reaction mixture was stirred at rt for 4 h, it was concentrated and purified by silica gel chromatography (petroleum ether/EtOAc=5/1 to 1/1) to provide the title compound (1.6 g, 88.6% yield) which was used directly in the next step.

Step 5: Synthesis of tert-butyl 7-(tosyloxy)-4-azaspiro[2.5]octane-4-carboxylate

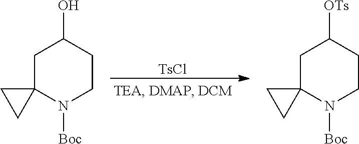 [SMILES: CC(C)(C)OC(=O)N1CCC(O)CC12CC2 ; "TsCl" ; Cc1ccc(S(=O)(=O)OC2CCN(C(=O)OC(C)(C)C)C3(CC3)C2)cc1]

To a solution of tert-butyl 7-hydroxy-4-azaspiro[2.5]octane-4-carboxylate (140 mg, 615.93 μmol) in TEA (3 mL) and DCM (10 mL) were added DMAP (75.25 mg, 615.93 μmol) and TsCl (176.46 mg, 923.89 μmol) at rt. After the reaction mixture was stirred at rt for 2 h, it was concentrated and purified by silica gel chromatography (petroleum ether/EtOAc=10/1 to 5/1) to provide the title compound (200 mg, 85.1% yield) as a yellow solid. MS (ESI) m/z=282.0 [M−Boc+H]⁺.

Step 6: Synthesis of tert-butyl 7-(4-(quinoxalin-2-yl)-1H-pyrazol-1-yl)-4-azaspiro[2.5]octane-4-carboxylate

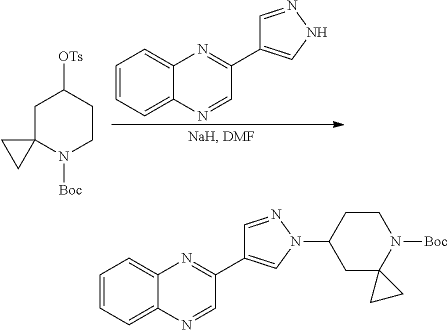

To a solution of 2-(1H-pyrazol-4-yl)quinoxaline (154.30 mg, 786.40 μmol) in DMF (10 mL) was added NaH (30.13 mg, 786.40 μmol) at rt. The mixture was stirred at rt for another 0.5 h, before a solution of tert-butyl 7-(p-tolylsulfonyloxy)-4-azaspiro[2.5]octane-4-carboxylate (200 mg, 524.27 μmol) in DMF (2 mL) was added. The resulting mixture was stirred at 85° C. for 2 h, before it was purified by prep-HPLC to provide the title compound (120 mg, 56. 5% yield). MS (ESI) m/z=406.4 [M+H]⁺.

Step 7: Synthesis of 2-(1-(4-azaspiro[2.5]octan-7-yl)-1H-pyrazol-4-yl)quinoxaline

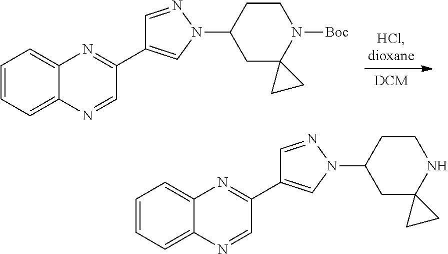 [SMILES: CC(C)(C)OC(=O)N1CCC(n2cc(-c3cnc4ccccc4n3)cn2)CC12CC2 ; c1ccc2nc(-c3cnn(C4CCNC5(CC5)C4)c3)cnc2c1]

To a solution of tert-butyl 7-(4-quinoxalin-2-ylpyrazol-1-yl)-4-azaspiro[2.5]octane-4-carboxylate (120 mg, 295.94 μmol) in DCM (5 mL) was added HCl/dioxane (1 M, 4 mL) at rt. After the reaction mixture was stirred at rt for 1 h, it was concentrated to provide the title compound (100 mg, 98.9% yield). MS (ESI) m/z=306.1 [M+H]⁺.

Step 8: Synthesis of 2-(2,6-dioxopiperidin-3-yl)-5-((5-oxo-5-(7-(4-(quinoxalin-2-yl)-1H-pyrazol-1-yl)-4-azaspiro[2.5]octan-4-yl)pentyl)amino)isoindoline-1,3-dione

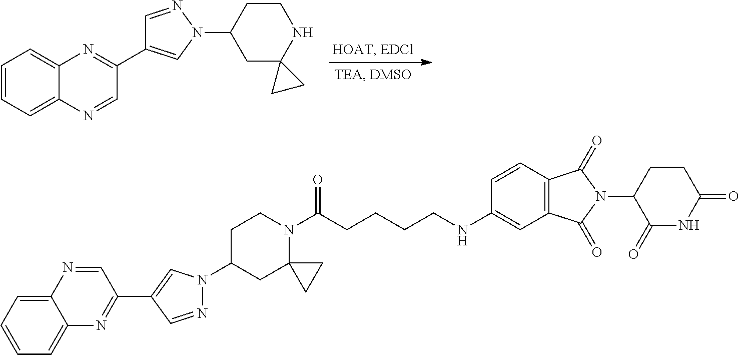 [SMILES: c1ccc2nc(-c3cnn(C4CCNC5(CC5)C4)c3)cnc2c1 ; O=C1CCC(N2C(=O)c3ccc(NCCCCC(=O)N4CCC(n5cc(-c6cnc7ccccc7n6)cn5)CC45CC5)cc3C2=O)C(=O)N1]

To a solution of 2-[1-(4-azaspiro[2.5]octan-7-yl)pyrazol-4-yl]quinoxaline (100 mg, 264.34 μmol) and 5-[[2-(2,6-dioxo-3-piperidyl)-1,3-dioxo-isoindolin-5-yl]amino]pentanoic acid (98.69 mg, 264.34 μmol) in DMSO (10 mL) were added HOAt (53.53 mg, 396.51 μmol), EDCI (76.13 mg, 396.51 μmol) and TEA (160.49 mg, 1.59 mmol) at rt. After the reaction mixture was stirred at rt for 16 h, it was purified by prep-HPLC to provide crude product which was further purified by prep-TLC (DCM/MeOH=15/1) to provide the title compound (75 mg, 42.9% yield). MS (ESI) m/z=661.5 [M+H]⁺.

### Example 112. 3-(1-Oxo-4-((3-(4-oxo-4-(4-(4-(quinoxalin-2-yl)-1H-pyrazol-1-yl)piperidin-1-yl)butyl)cyclohexyl)amino)isoindolin-2-yl)piperidine-2,6-dione (GS-154)

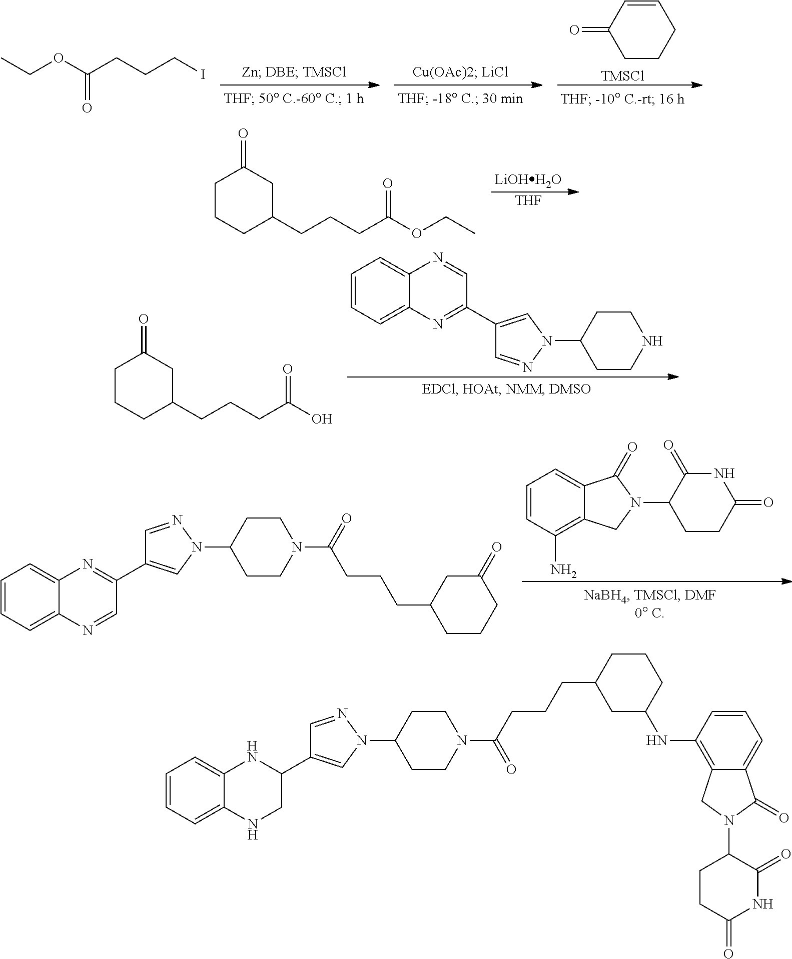

Step 1: Synthesis of ethyl 4-(3-oxocyclohexyl)butanoate

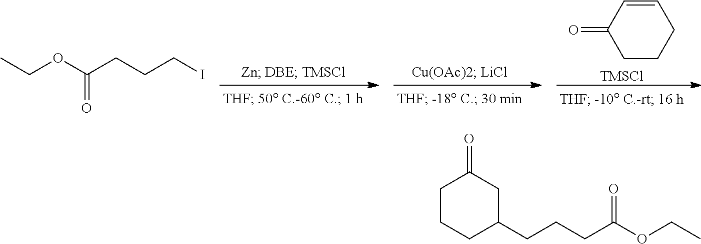

Under nitrogen atmosphere, a mixture of zinc (374 mg, 5.72 mmol) 1,2-dibromoethane (44 mg, 0.234 mmol, 0 09 mL) and THF (2 mL) in a three-neck flask was heated to 60° C.˜ 70° C. for a minute and then cooled to rt. Chlorotrimethylsilane (19 mg, 0.18 mmol) was added, and the mixture was stirred at rt for 15 min. A solution of ethyl 4-iodobutanoate (1.3 g, 5.5 mmol) in THF (3 ml) was then added and the mixture was heated with heat gun. The resulting solution of (4-ethoxy-4-oxobutyl)zinc(II) iodide in THF was ready to use. Anhydrous Cu(OAc)₂ (520 mg, 2.86 mmol), LiCI (243 mg, 5.73 mmol) and THF (4 mL) were put into another three-neck flask charged with nitrogen gas. The mixture was stirred at rt for 30 min before being cooled to −18° C. in salt-ice bath. The solution of (4-ethoxy-4-oxobutyl)zinc(II) iodide in THF was added dropwise. After completion of addition, the mixture was stirred at −18° C. for 10 min. A solution of cyclohex-2-en-1-one (250 mg, 2.6 mmol) and chlorotrimethylsilane (5.21 mmol, 0.66 mL) in THF (2.5 mL) was added over 20 min. The reaction mixture was stirred at −18° C.˜−10° C. for 1 h before it was warmed to rt. After being stirred overnight, the mixture was quenched with saturated aqueous NH₄Cl and extracted with EtOAc. The combined organic layer was dried over Na₂SO₄, and concentrated to provide crude product (743 mg, 40.4% yield) as a brown oil which was used directly in the next step without further purification.

Step 2: Synthesis of 4-(3-oxocyclohexyl)butanoic acid

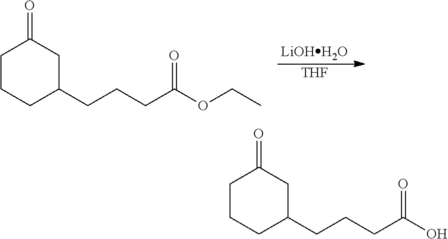

A mixture of ethyl 4-(3-oxocyclohexyl)butanoate (743 mg, 1.05 mmol), LiOH·H₂O (220.50 mg, 5.25 mmol) in THF (8 mL) and H₂O (2 mL) was stirred at 25° C. for 2 h. After THF was removed, the resulting residue was diluted with water. After the pH of the mixture was adjusted to ˜3 with 1N HCl, the mixture was extracted with EtOAc. The organic layers were combined, dried, concentrated to provide the title compound (183 mg, 94.6% yield) as a brown oil which was used directly in the next step without further purification.

Step 3: Synthesis of 3-(4-oxo-4-(4-(4-(quinoxalin-2-yl)-1H-pyrazol-1-yl)piperidin-1-yl)butyl)cyclohexan-1-one

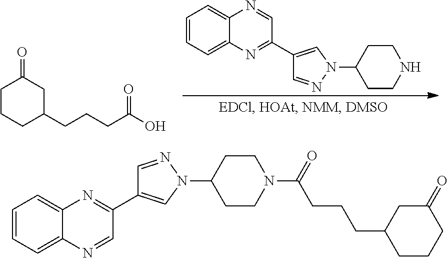

To a solution of 4-(3-oxocyclohexyl)butanoic acid (168 mg, 0.91 mmol) of DMSO (5 mL) were added HOAt (246.21 mg, 1.82 mmol), EDCI (350.17 mg, 1.82 mmol) and NMM (461.19 mg, 4.56 mmol). The mixture was stirred at rt for 2 min, before 2-[1-(4-piperidyl)pyrazol-4-yl]quinoxaline (254.73 mg, 0.91 mmol) was added. After the reaction was stirred at 25° C. for 12 h, it was purified by reverse phase column, followed by prep-TLC (DCM/MeOH=15:1) to provide the title compound (64 mg, 15.8% yield) as a white solid. MS (ESI) m/z=446.3 [M+H]⁺.

Step 4: Synthesis of 3-(1-oxo-4-((3-(4-oxo-4-(4-(4-(1,2,3,4-tetrahydroquinoxalin-2-yl)-1H-pyrazol-1-yl)piperidin-1-yl)butyl)cyclohexyl)amino)isoindolin-2-yl)piperidine-2,6-dione

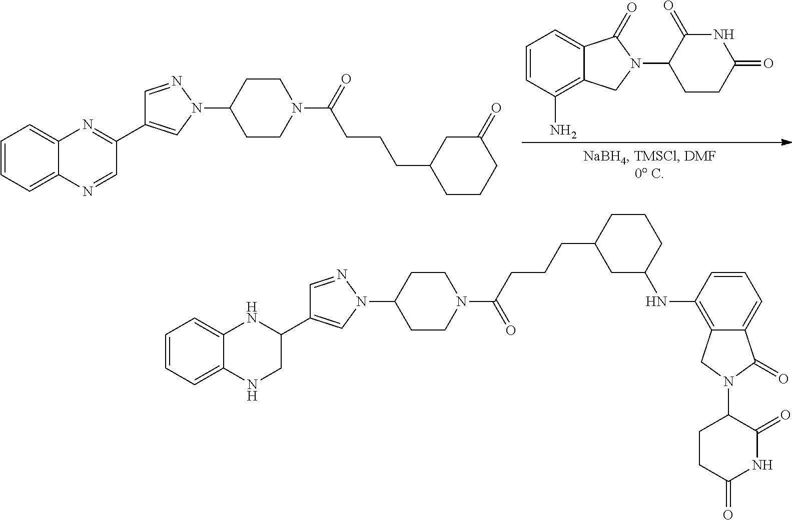

A mixture of 3-[4-oxo-4-[4-(4-quinoxalin-2-ylpyrazol-1-yl)-1-piperidyl]butyl]cyclohexanone (25 mg, 0.056 mmol) and 3-(4-amino-1-oxo-isoindolin-2-yl)piperidine-2,6-dione (19 mg, 0.073 mmol) in DMF (1.5 mL) was stirred at 0° C. for 2 min, at which time, NaBH₄ (3.20 mg, 84.16 μmol) was added. The reaction was stirred at 0° C. for 40 min, before TMSCl (24 mg, 0.22 mmol) was added. After the reaction was stirred for another 30 min, the reaction was concentrated to provide the crude product (38 mg, 97% yield) which was used directly in the next step. MS (ESI) m/z=693.6 [M+H]⁺.

Step 5: Synthesis of 3-(1-oxo-4-((3-(4-oxo-4-(4-(4-(quinoxalin-2-yl)-1H-pyrazol-1-yl)piperidin-1-yl)butyl)cyclohexyl)amino)isoindolin-2-yl)piperidine-2,6-dione

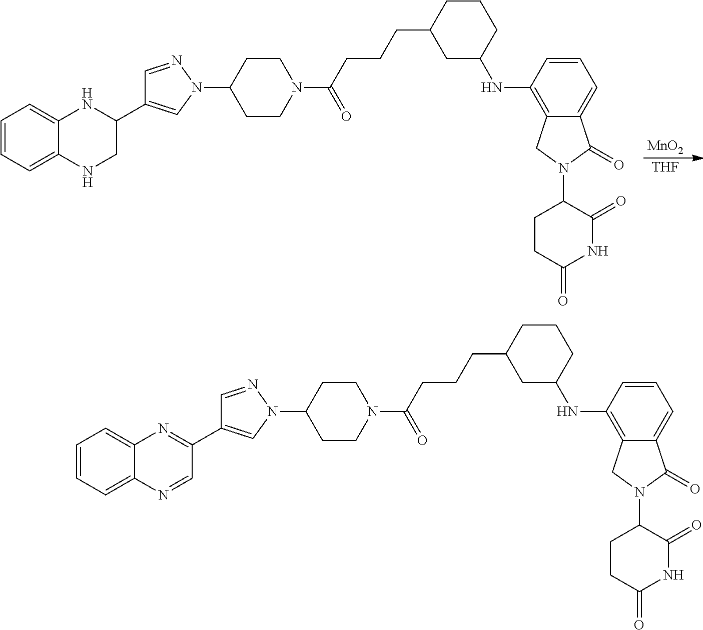

A mixture of 3-[1-oxo-4-[[3-[4-oxo-4-[4-[4-(1,2,3,4-tetraydroquinoxalin-2-yl)pyrazol-1-yl]-1-piperidyl]butyl]cyclohexyl]amino]isoindolin-2-yl]piperidine-2,6-dione (38 mg, 0.55 mmol) and MnO₂ (24 mg, 0.27 mmol) in THF (5 mL) was stirred at 0° C. for 0.5 h. The mixture was purified by reverse phase column, followed by prep-TLC (DCM/MeOH=13.5:1) to provide the title compound (17 mg, 45% yield) as a yellow solid. MS (ESI) m/z=689.4 [M+H]⁺.

### Example 113. 2-(2,6-Dioxopiperidin-3-yl)-5-(((4-((4-(quinoxalin-2-yl)-1H-pyrazol-1-yl)methyl)cyclohexyl)methyl)amino)isoindoline-1,3-dione (GS-155)

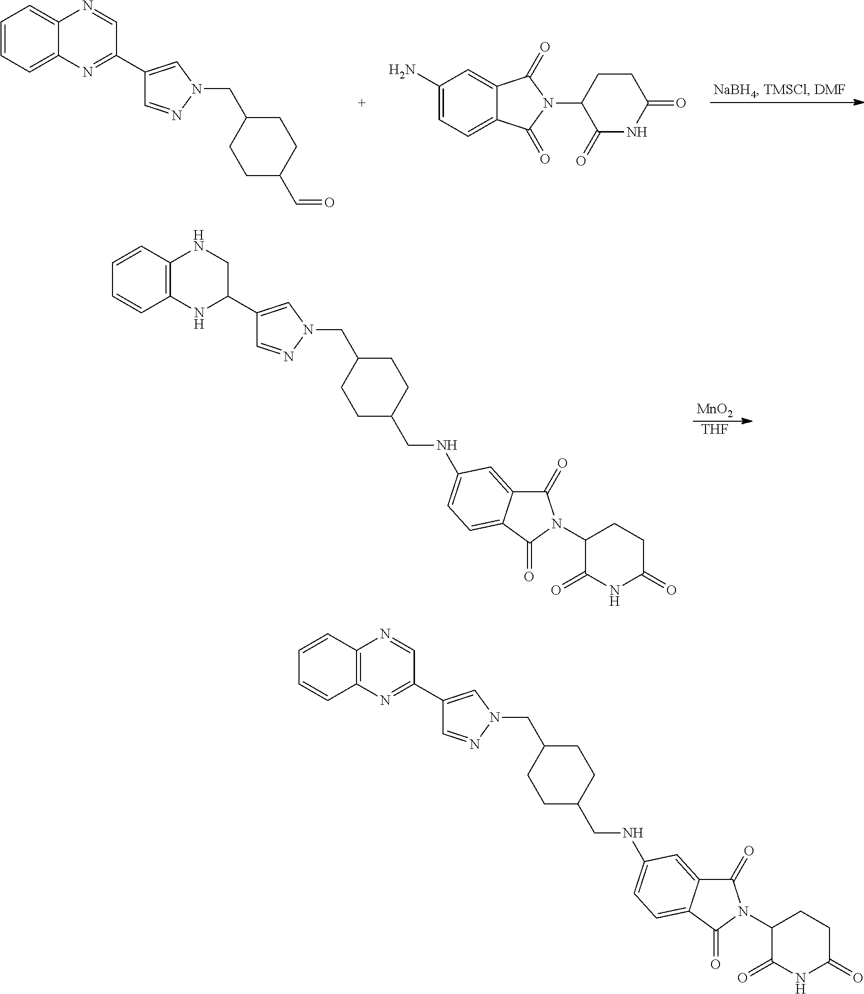

GS-155 was synthesized following the similar procedure or preparing GS-154 (16 mg, yield: 22.4%). MS (ESI) m/z=578.4 [M+H]⁺.

### Example 114. 2-(2,6-Dioxopiperidin-3-yl)-5-((3-(3-(4-(8-fluoroquinoxalin-2-yl)-3,5-dimethyl-1H-pyrazol-1-yl)cyclobutyl)propyl)amino)isoindoline-1,3-dione (GS-156)

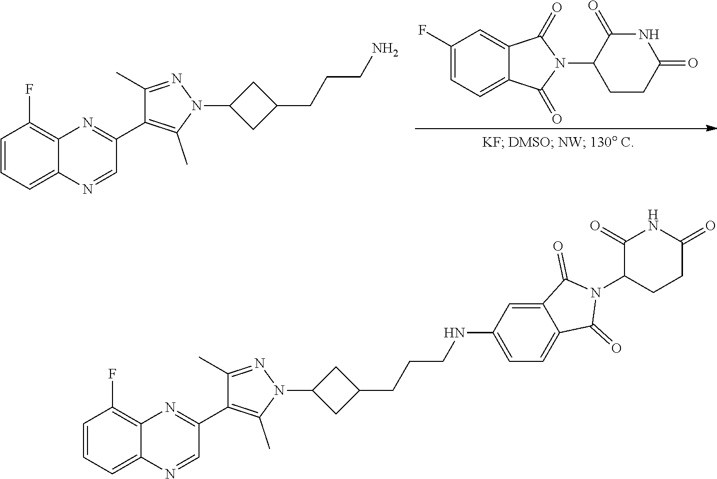 [SMILES: Cc1nn(C2CC(CCCN)C2)c(C)c1-c1cnc2cccc(F)c2n1 ; O=C1CCC(N2C(=O)c3ccc(F)cc3C2=O)C(=O)N1 ; Cc1nn(C2CC(CCCNc3ccc4c(c3)C(=O)N(C3CCC(=O)NC3=O)C4=O)C2)c(C)c1-c1cnc2cccc(F)c2n1]

GS-156 was synthesized following the similar procedure for preparing GS-107 (36.5 mg, yield: 19.3%). MS (ESI) m/z=610.3 [M+H]⁺.

### Example 115. 3-(1-Oxo-4-((3-(3-(4-(quinoxalin-2-yl)-1H-pyrazol-1-ylpropyl)cyclohexyl)amino)isoindolin-2-yl)piperidine-2,6-dione (GS-157)

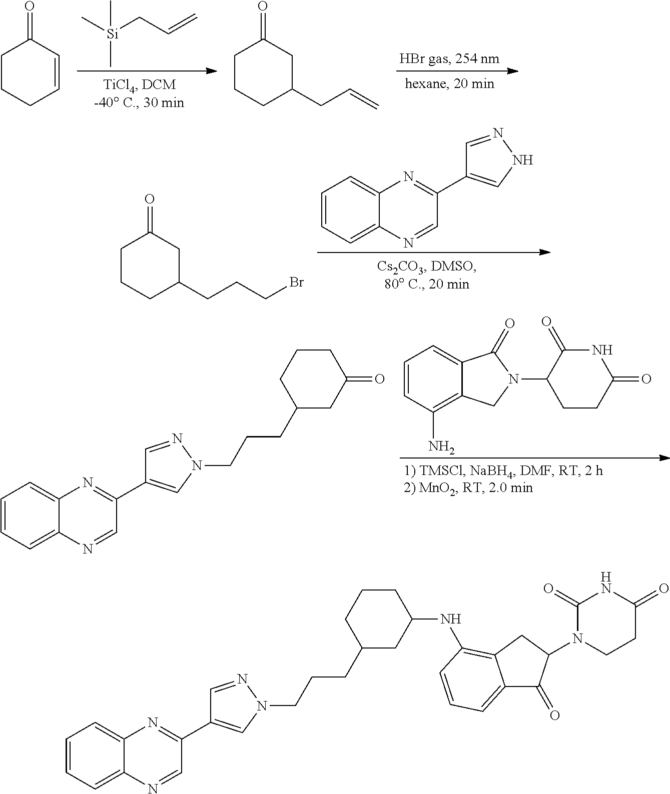

Step 1: Synthesis of 3-allylcyclohexan-1-one

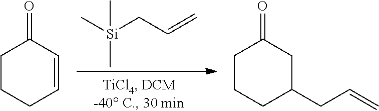

To a stirred solution of cyclohex-2-en-1-one (250 mg, 2.6 mmol) in DCM (2.5 mL) was added TiCl₄ (0.29 mL, 2.6 mmol) under N₂ at −40° C. The reaction was stirred at −40° C. for 10 min, before a solution of allyltrimethylsilane (0.54 mL, 3.39 mmol) in DCM (4.0 mL) was added dropwise. The resulting mixture was stirred at −40° C. for 30 min, before it was quenched with H₂O and extracted with EtOAc (20 mL×3). The organic layers were combined, washed standard NaHCO₃ and brine, dried over Na₂SO₄, filtered, and concentrated to provide the crude title compound (160 mg, 44.6% yield) as colorless oil which was used directly in the next step without further purification.

Step 2: Synthesis of 3-(3-bromopropyl)cyclohexan-1-one

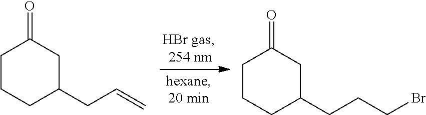

A solution of 3-allylcyclohexan-1-one (160 mg crude, 1.16 mmol) in hexane (5.0 mL) was irradiated by UV light (254 nm), while HBr gas was slowly bubbled into the solution. The reaction was stirred at rt for 20 min, before it was quenched with standard Na₂S2O₃. The organic layer was washed with standard NaHCO₃ and brine, dried over Na₂SO₄, filtered, and concentrated to provide the crude title compound (108 mg, 42.5% yield) as a yellow oil which was used directly in the next step without further purification.

Step 3: Synthesis of 3-(3-(4-(quinoxalin-2-yl)-1H-pyrazol-1-yl)propyl)cyclohexan-1-one

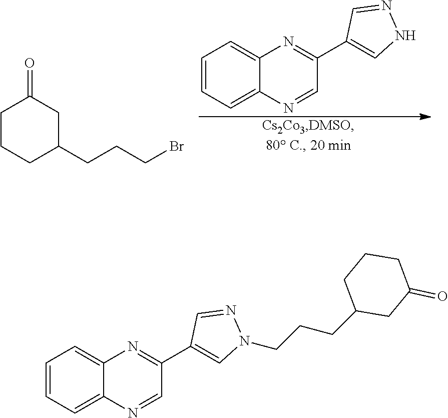

To a solution of 2-(1H-pyrazol-4-yl)quinoxaline (80 mg, 0.41 mmol) and 3-(3-bromopropyl)cyclohexan-1-one (108 mg crude, 0.49 mmol) in DMSO (1.5 mL) was added Cs₂CO₃ (266 mg, 0.81 mmol). The reaction was stirred at 80° C. for 20 min, before it was cooled to rt and purified by reverse phase column chromatography to provide the title compound (18 mg, 13.1% yield) as a light-yellow solid. MS (ESI) m/z=335.2 [M+H]⁺.

Step 4: Synthesis of 3-(1-oxo-4-((3-(3-(4-(quinoxalin-2-yl)-1H-pyrazol-1-yl)propyl)cyclohexyl)amino)isoindolin-2-yl)piperidine-2,6-dione

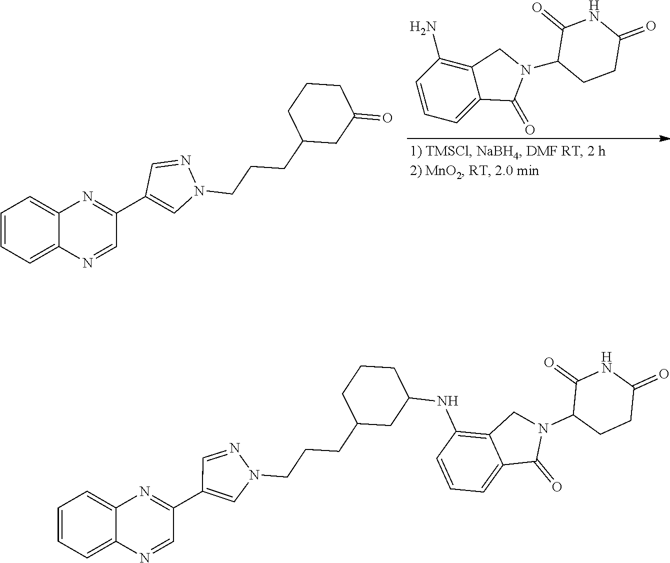

GS-157 was synthesized following the similar procedure for preparing GS-154 (14.4 mg, yield: 45%). MS (ESI) m/z=578.5 [M+H]⁺.

### Example 116. 3-(1-Oxo-6-(4-oxo-4-(4-(4-(quinoxalin-2-yl)-1H-pyrazol-1-yl)piperidin-1-yl)butyl)isoindolin-2-yl)piperidine-2,6-dione (GS-158)

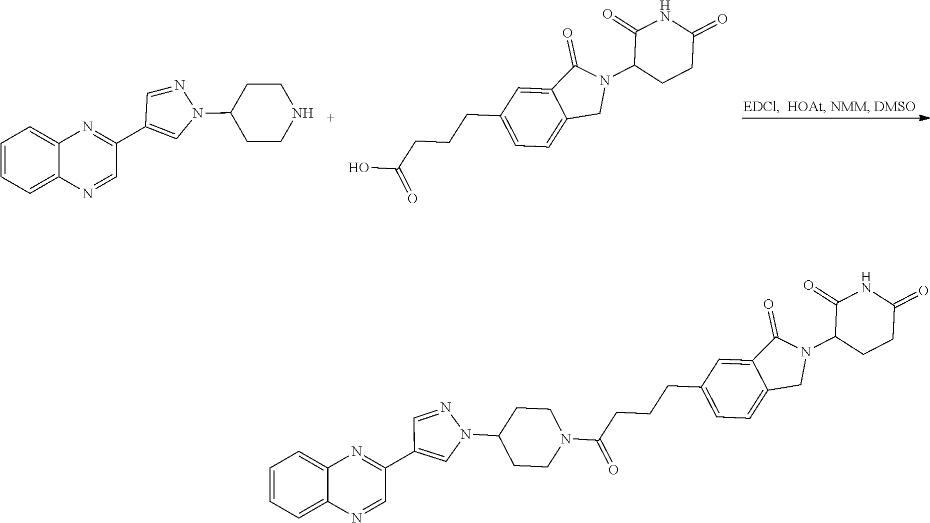 [SMILES: c1ccc2nc(-c3cnn(C4CCNCC4)c3)cnc2c1 ; O=C(O)CCCc1ccc2c(c1)C(=O)N(C1CCC(=O)NC1=O)C2 ; O=C1CCC(N2Cc3ccc(CCCC(=O)N4CCC(n5cc(-c6cnc7ccccc7n6)cn5)CC4)cc3C2=O)C(=O)N1]

GS-158 was synthesized following the similar procedure for preparing GS-001 (7.4 mg, yield: 32.7%). MS (ESI) m/z=592.4 [M+H]⁺.

### Example 117. 3-(1-Oxo-6-(4-oxo-4-(4-(4-(quinolin-2-yl)-1H-pyrazol-1-yl)piperidin-1-yl)butyl)isoindolin-2-yl)piperidine-2,6-dione (GS-159)

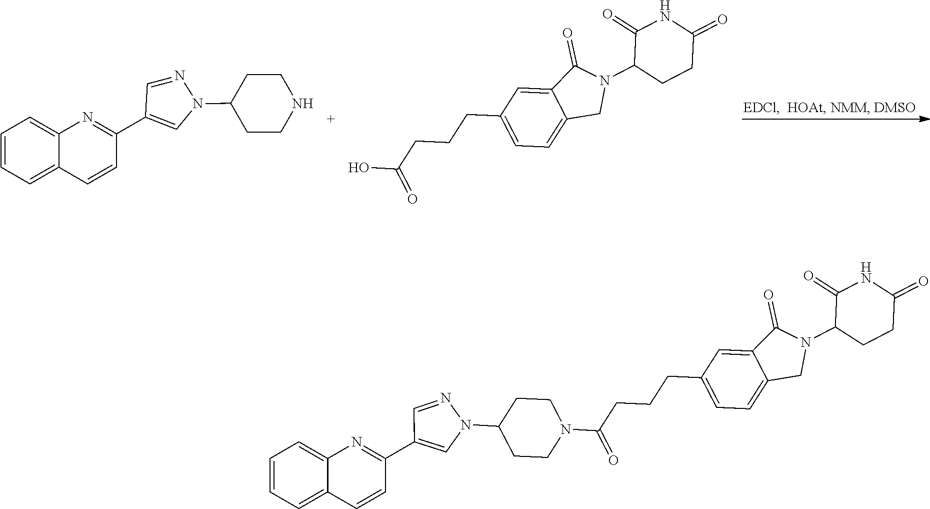 [SMILES: c1ccc2nc(-c3cnn(C4CCNCC4)c3)ccc2c1 ; O=C(O)CCCc1ccc2c(c1)C(=O)N(C1CCC(=O)NC1=O)C2 ; O=C1CCC(N2Cc3ccc(CCCC(=O)N4CCC(n5cc(-c6ccc7ccccc7n6)cn5)CC4)cc3C2=O)C(=O)N1]

GS-159 was synthesized following the similar procedure for preparing GS-001 (3.8 mg, yield: 12.7%). MS (ESI) m/z=591.5 [M+H]⁺.

### Example 118. 3-(1-Oxo-6-(5-oxo-5-(4-(4-(quinolin-2-yl)-1H-pyrazol-1-yl)piperidin-1-yl)pentyl)isoindolin-2-yl)piperidine-2,6-dione (GS-160)

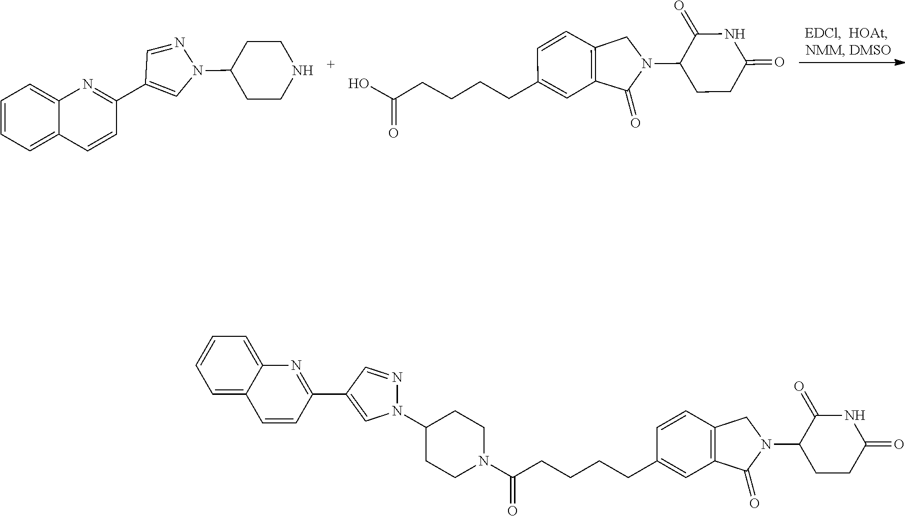 [SMILES: c1ccc2nc(-c3cnn(C4CCNCC4)c3)ccc2c1 ; O=C(O)CCCCc1ccc2c(c1)C(=O)N(C1CCC(=O)NC1=O)C2 ; O=C1CCC(N2Cc3ccc(CCCCC(=O)N4CCC(n5cc(-c6ccc7ccccc7n6)cn5)CC4)cc3C2=O)C(=O)N1]

GS-160 was synthesized following the similar procedure for preparing GS-001 (18.2 mg, yield: 34.6%). MS (ESI) m/z=605.5 [M+H]⁺.

### Example 119. 3-(1-Oxo-6-(5-oxo-5-(4-(4-(quinoxalin-2-yl)-1H-pyrazol-1-yl)piperidin-1-yl)pentyl)isoindolin-2-yl)piperidine-2,6-dione (GS-161)

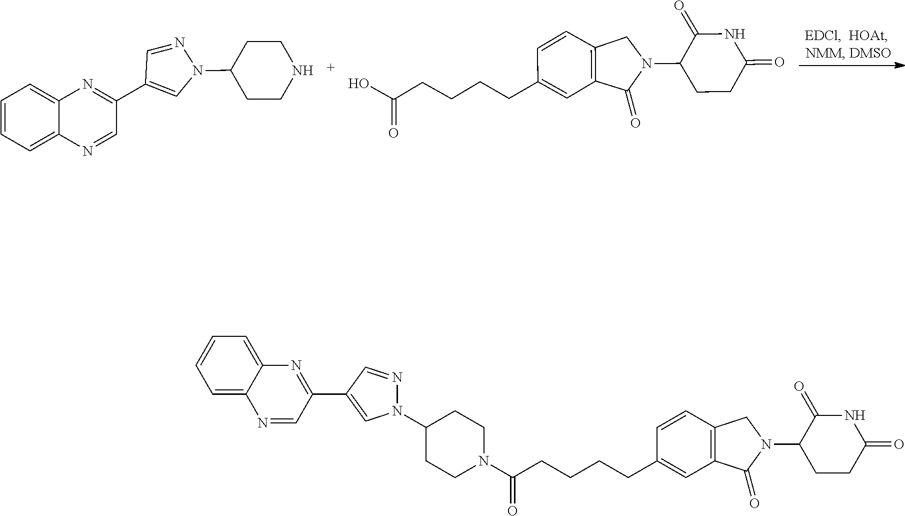 [SMILES: c1ccc2nc(-c3cnn(C4CCNCC4)c3)cnc2c1 ; O=C(O)CCCCc1ccc2c(c1)C(=O)N(C1CCC(=O)NC1=O)C2 ; O=C1CCC(N2Cc3ccc(CCCCC(=O)N4CCC(n5cc(-c6cnc7ccccc7n6)cn5)CC4)cc3C2=O)C(=O)N1]

GS-161 was synthesized following the similar procedure for preparing GS-001 (16.4 mg, yield: 31%). MS (ESI) m/z=606.5 [M+H]⁺.

### Example 120. 3-(1-Oxo-5-((3-(4-(4-(quinoxalin-2-yl)-1H-pyrazol-1-yl)piperidin-1-yl)benzyl)amino)isoindolin-2-yl)piperidine-2,6-dione (GS-162)

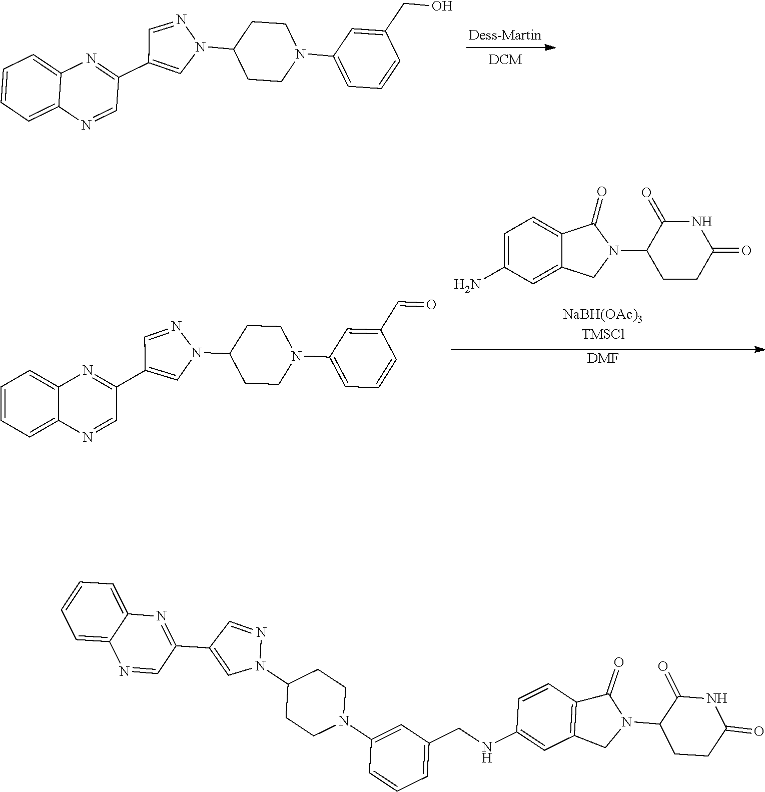

Step 1: Synthesis of 3-(4-(4-(quinoxalin-2-yl)-1H-pyrazol-1-yl)piperidin-1-yl)benzaldehyde

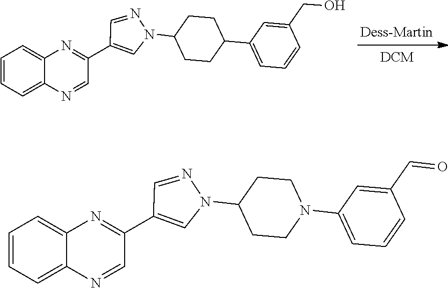 [SMILES: OCc1cccc(C2CCC(n3cc(-c4cnc5ccccc5n4)cn3)CC2)c1 ; O=Cc1cccc(N2CCC(n3cc(-c4cnc5ccccc5n4)cn3)CC2)c1]

To a solution of (3-(4-(4-(quinoxalin-2-yl)-1H-pyrazol-1-yl)piperidin-1-yl)phenyl)methanol (50 mg, 0.13 mmol) in DCM (3 mL) was added 1,1,1-tris(acetyloxy)-1,1-dihydro-1,2-benzodioxol-3-(1H)-one (124 mg, 0.30 mmol) at 0° C. The reaction was stirred at rt under nitrogen for 1 h, before it was diluted with DCM, washed with brine, dried over Na₂SO₄, filtered, and concentrated to yield the crude product (42 mg) which was used directly in the next step. MS (ESI) m/z=384.3 [M+H]⁺.

Step 2: Synthesis of 3-(1-oxo-5-((3-(4-(4-(quinoxalin-2-yl)-1H-pyrazol-1-yl)piperidin-1-yl)benzyl)amino)isoindolin-2-yl)piperidine-2,6-dione

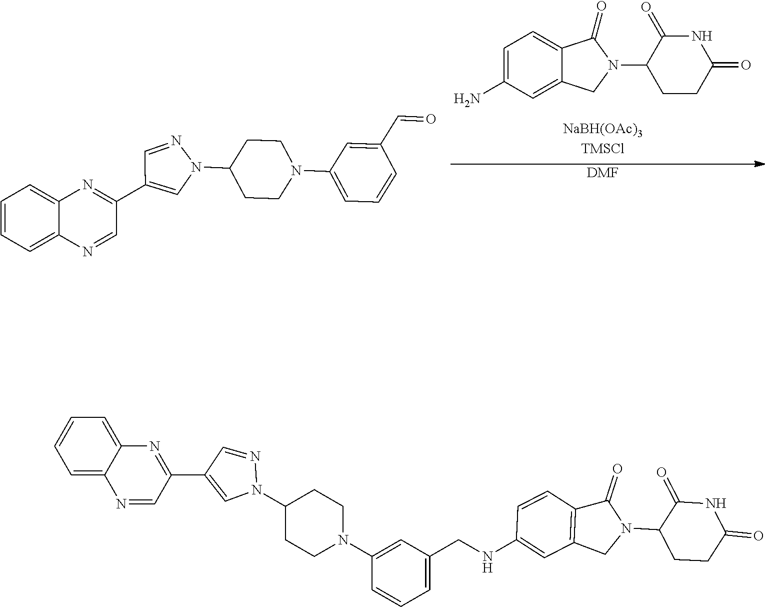

To a solution of 3-(4-(4-(quinoxalin-2-yl)-1H-pyrazol-1-yl)piperidin-1-yl)benzaldehyde (42 mg, 0.11 mmol) and 3-(5-amino-1-oxoisoindolin-2-yl)piperidine-2,6-dione (28.4 mg, 0.11 mmol) in DMF (3 mL) was added TMSCl (1.18 mg, 0.01 mmol) dropwise at 0° C. The reaction mixture was stirred at 0° C. for 30 min. Then a solution of NaBH(OAc)₃ (46.4 mg, 0.22 mmol) in 1 mL DMF was added dropwise. After the mixture was stirred for another 30 min, the reaction was quenched with water (5 mL), and extracted with EtOAc (2×5 mL). The combined organic layers were washed with brine (10 mL), dried over anhydrous Na₂SO4, and concentrated to yield a crude product which was directly purified by silica gel column chromatography to provide the title compound (20.2 mg, 29.4%) as a yellow solid. MS (ESI) m/z=627.6 [M+H]⁺.

### Example 121. 3-(1-(6-((2-(2,6-Dioxopiperidin-3-yl)-1,3-dioxoisoindolin-5-yl)amino)hexyl)-1H-pyrazol-4-yl)quinoxaline-6-carbonitrile (GS-163)

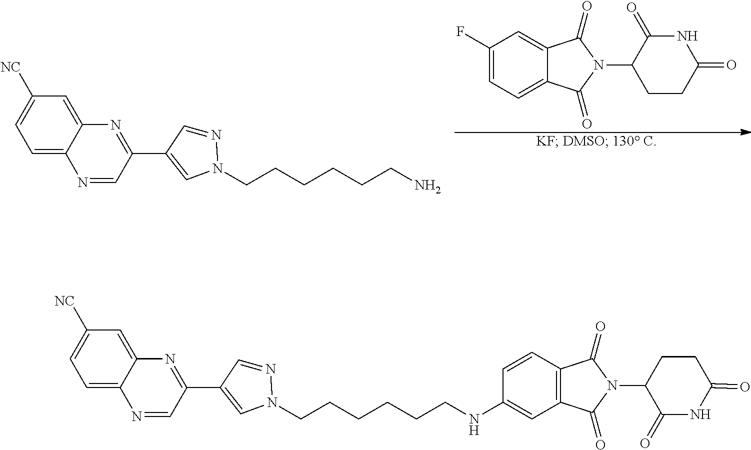 [SMILES: N#Cc1ccc2ncc(-c3cnn(CCCCCCN)c3)nc2c1 ; O=C1CCC(N2C(=O)c3ccc(F)cc3C2=O)C(=O)N1 ; "KF; DMSO; 130° C." ; N#Cc1ccc2ncc(-c3cnn(CCCCCCNc4ccc5c(c4)C(=O)N(C4CCC(=O)NC4=O)C5=O)c3)nc2c1]

GS-163 was synthesized following the similar procedure for preparing GS-107 (10.2 mg, yield: 25.3%). MS (ESI) m/z=577.4 [M+H]⁻.

### Example 122. 2-(2,6-Dioxopiperidin-3-yl)-5-((3-(3-(4-(8-fluoroquinoxalin-2-yl)-1H-pyrazol-1-yl)cyclobutyl)propyl)amino)isoindoline-1,3-dione (GS-164)

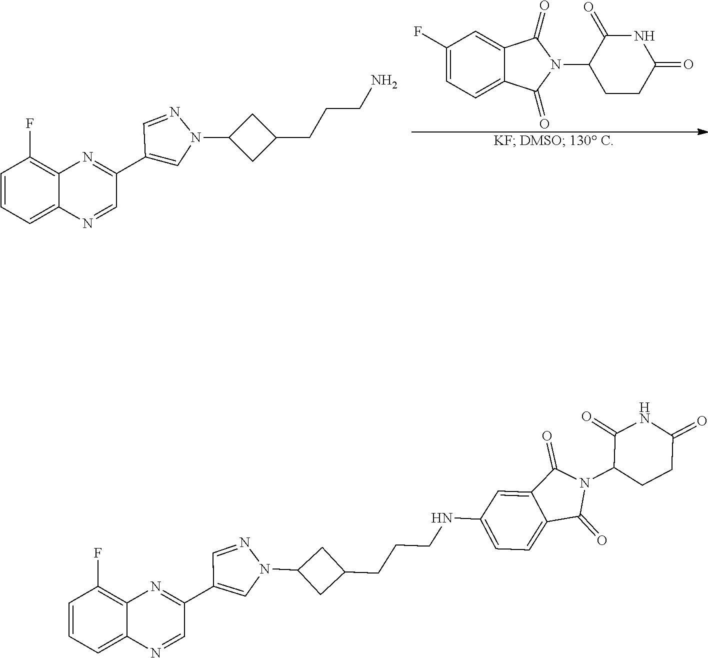 [SMILES: NCCCC1CC(n2cc(-c3cnc4cccc(F)c4n3)cn2)C1 ; O=C1CCC(N2C(=O)c3ccc(F)cc3C2=O)C(=O)N1 ; "KF; DMSO; 130° C." ; O=C1CCC(N2C(=O)c3ccc(NCCCC4CC(n5cc(-c6cnc7cccc(F)c7n6)cn5)C4)cc3C2=O)C(=O)N1]

GS-164 was synthesized following the similar procedure for preparing GS-107 (55 mg, yield: 16.6%). MS (ESI) m/z=582.4 [M+H]⁺.

### Example 123. 3-(1-(6-((2-(2,6-Dioxopiperidin-3-yl)-1,3-dioxoisoindolin-5-yl)amino)hexyl)-1H-pyrazol-4-yl)quinoxaline-5-carbonitrile (GS-165)

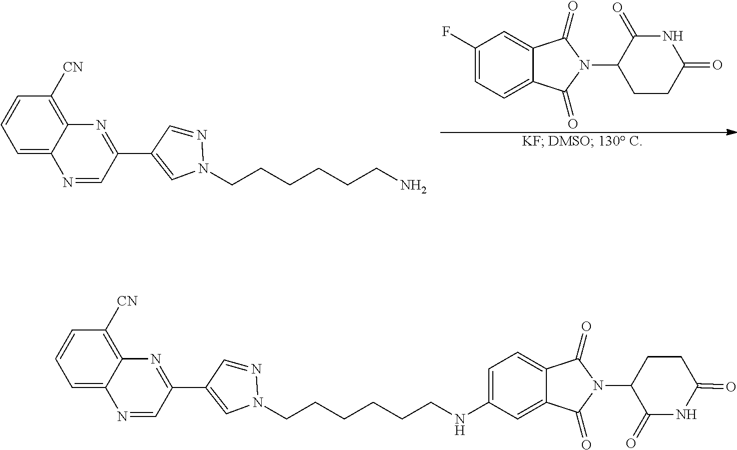 [SMILES: N#Cc1cccc2ncc(-c3cnn(CCCCCCN)c3)nc12 ; O=C1CCC(N2C(=O)c3ccc(F)cc3C2=O)C(=O)N1 ; "KF; DMSO; 130° C." ; N#Cc1cccc2ncc(-c3cnn(CCCCCCNc4ccc5c(c4)C(=O)N(C4CCC(=O)NC4=O)C5=O)c3)nc12]

GS-165 was synthesized following the similar procedure for preparing GS-107 (3.17 mg, yield: 8.8%). MS (ESI) m/z=577.4 [M+H]⁺.

### Example 124. 3-(1-(6-((2-(2,6-Dioxopiperidin-3-yl)-1,3-dioxoisoindolin-5-yl)amino)hexyl)-3,5-dimethyl-1H-pyrazol-4-yl)quinoxaline-5-carbonitrile (GS-166)

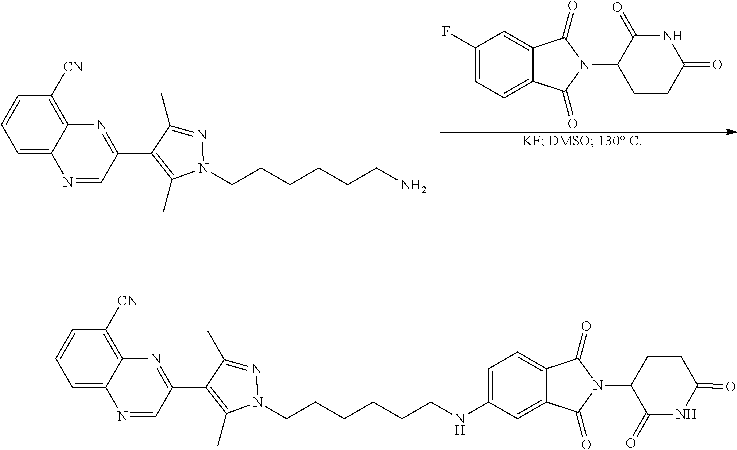 [SMILES: Cc1nn(CCCCCCN)c(C)c1-c1cnc2cccc(C#N)c2n1 ; O=C1CCC(N2C(=O)c3ccc(F)cc3C2=O)C(=O)N1 ; "KF; DMSO; 130° C." ; Cc1nn(CCCCCCNc2ccc3c(c2)C(=O)N(C2CCC(=O)NC2=O)C3=O)c(C)c1-c1cnc2cccc(C#N)c2n1]

GS-166 was synthesized following the similar procedure for preparing GS-107 (3.51 mg, yield: 10.1%). MS (ESI) m/z=605.4 [M+H]⁺.

### Example 125. 5-((3-(3-(3,5-Dimethyl-4-(quinoxalin-2-yl)-1H-pyrazol-1-yl)cyclobutyl)propyl)amino)-2-(2,6-dioxopiperidin-3-yl)isoindoline-1,3-dione (GS-167)

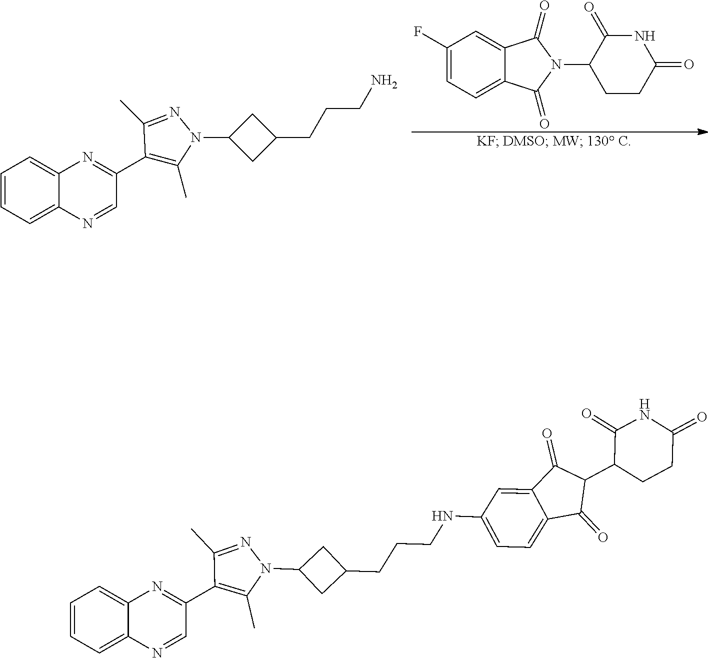

GS-167 was synthesized following the similar procedure for preparing GS-107 (44.9 mg, yield: 22.4%). MS (ESI) m/z=592.5 [M+H]⁺.

### Example 126. 2-(2,6-Dioxopiperidin-3-yl)-5-((3-(cis-3-(3-methyl-4-(quinoxalin-2-yl)-1H-pyrazol-1-yl)cyclobutyl)propyl)amino)isoindoline-1,3-dione (GS-168)

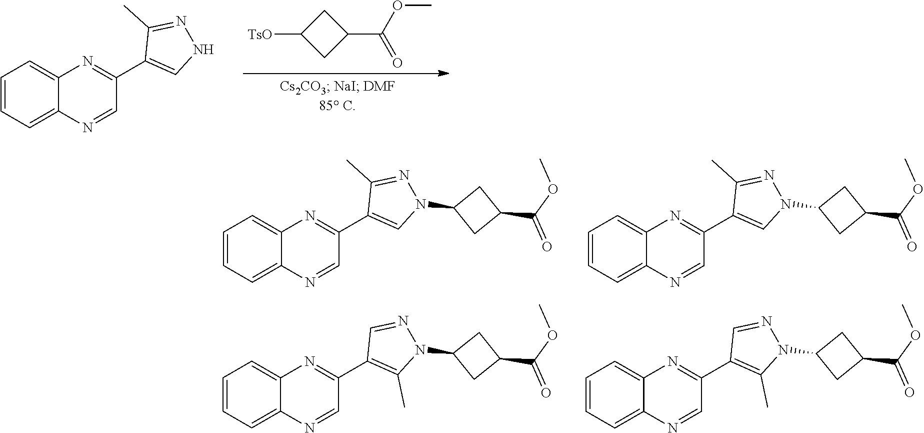

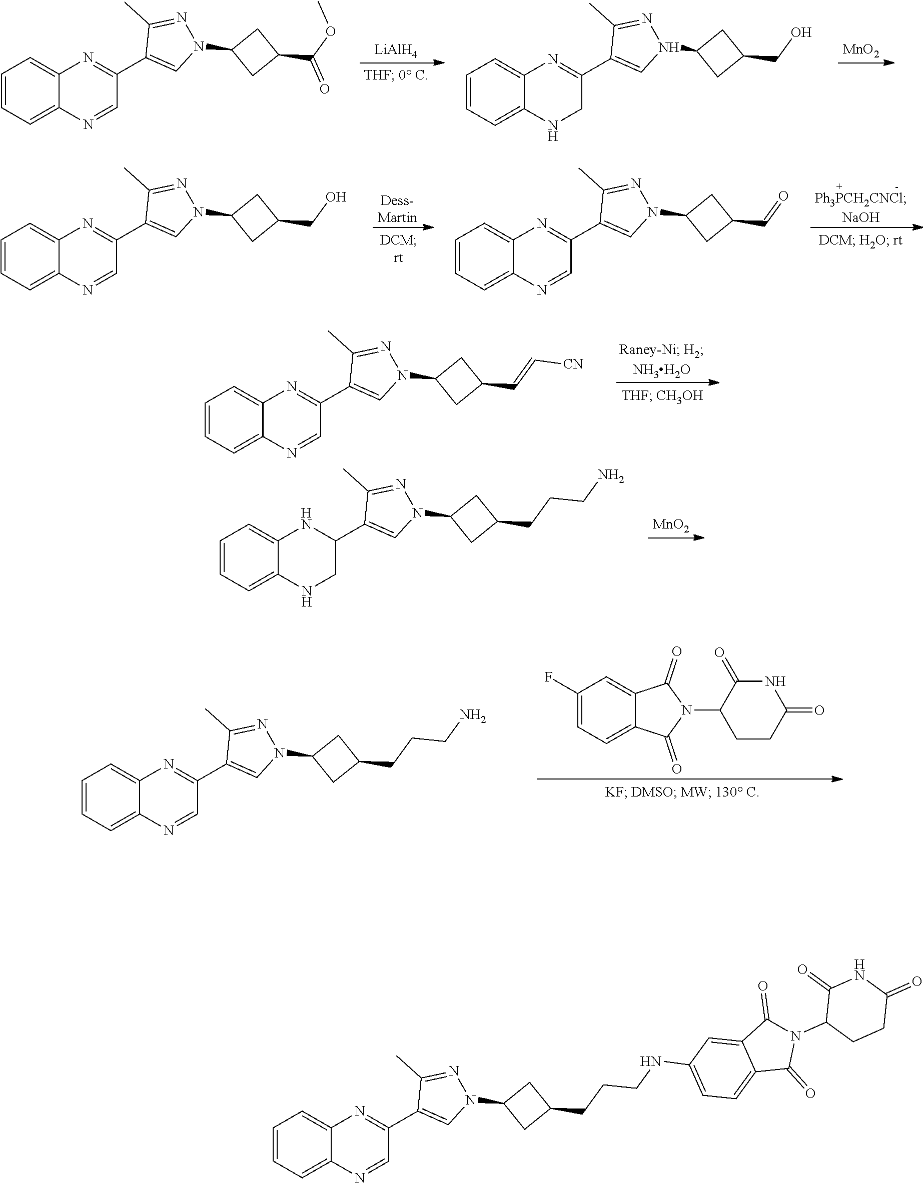

Step 1: Synthesis of methyl cis-3-(3-methyl-4-(quinoxalin-2-yl)-1H-pyrazol-1-yl)cyclobutane-1-carboxylate

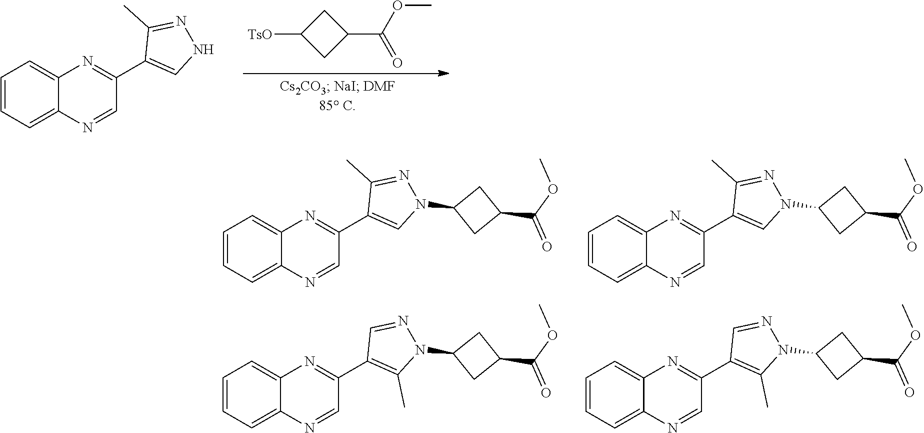

A mixture of methyl 2-(3-methyl-1H-pyrazol-4-yl)quinoxaline (1.5 g, 7.14 mmol), methyl 3-(tosyloxy)cyclobutane-1-carboxylate (3.04 g, 10.7 mmol), Cs₂CO₃ (6.98 g, 21.42 mmol) and NaI (1.61 g, 10.71 mmol) in DMF (15 mL) was stirred at 85° C. for 3 h. The reaction mixture was diluted with EtOAc (50 mL), washed with H₂O and brine, and concentrated. The resulting residue was purified by prep-HPLC to provide the title compound (285 mg, yield: 12.4%) as a pale solid. MS (ESI) m/z=323.3 [M+H]⁺.

Step 2: Synthesis of 2-(2,6-dioxopiperidin-3-yl)-5-((3-(cis-3-(3-methyl-4-(quinoxalin-2-yl)-1H-pyrazol-1-yl)cyclobutyl)propyl)amino)isoindoline-1,3-dione

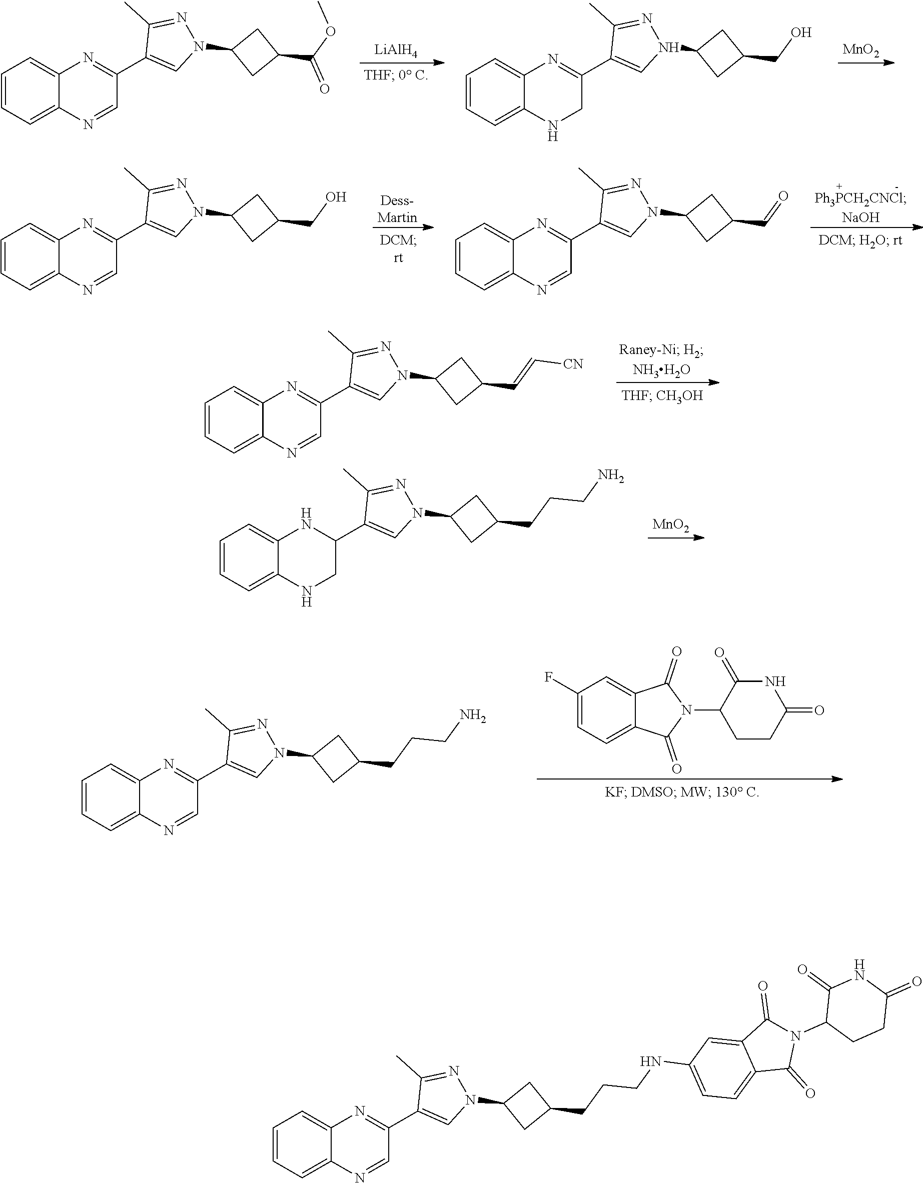

GS-168 was synthesized following the similar procedure for preparing GS-107 (8 mg, yield: 14.9%). MS (ESI) m/z=578.5 [M+H]⁺.

### Example 127. 2-(2,6-Dioxopiperidin-3-yl)-5-((3-(cis-3-(5-methyl-4-(quinoxalin-2-yl)-1H-pyrazol-1-yl)cyclobutyl)propyl)amino)isoindoline-1,3-dione (GS-169)

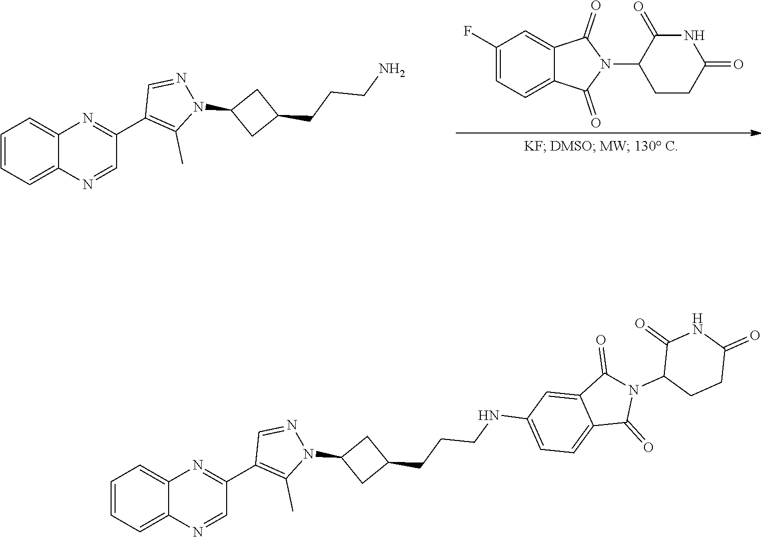 [SMILES: Cc1c(-c2cnc3ccccc3n2)cnn1[C@H]1C[C@@H](CCCN)C1 ; O=C1CCC(N2C(=O)c3ccc(F)cc3C2=O)C(=O)N1 ; Cc1c(-c2cnc3ccccc3n2)cnn1[C@H]1C[C@@H](CCCNc2ccc3c(c2)C(=O)N(C2CCC(=O)NC2=O)C3=O)C1]

GS-169 was synthesized following the similar procedure for preparing GS-168 (7 mg, yield: 20.20%). MS (ESI) m/z=578.5 [M+H]⁺.

### Example 128. 2-(2,6-Dioxopiperidin-3-yl)-5-((3-(trans-3-(5-methyl-4-(quinoxalin-2-yl)-1H-pyrazol-1-yl)cyclobutyl)propyl)amino)isoindoline-1,3-dione (GS-170)

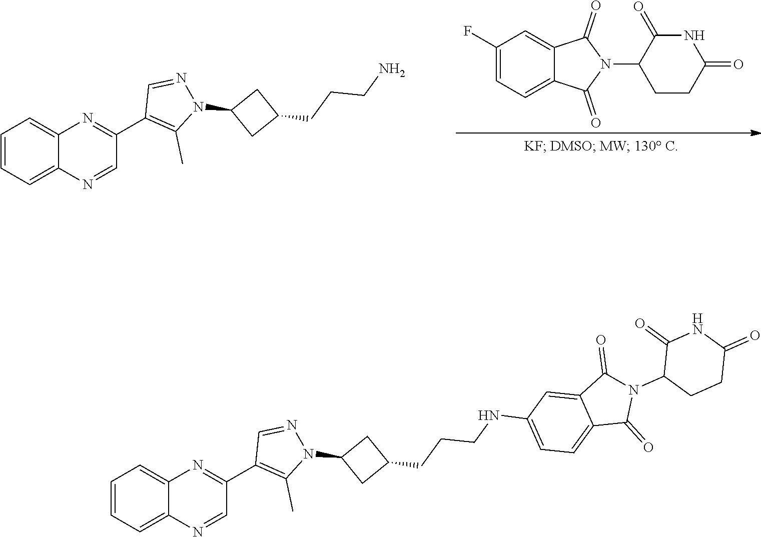 [SMILES: Cc1c(-c2cnc3ccccc3n2)cnn1[C@H]1C[C@H](CCCN)C1 ; O=C1CCC(N2C(=O)c3ccc(F)cc3C2=O)C(=O)N1 ; Cc1c(-c2cnc3ccccc3n2)cnn1[C@H]1C[C@H](CCCNc2ccc3c(c2)C(=O)N(C2CCC(=O)NC2=O)C3=O)C1]

GS-170 was synthesized following the similar procedure for preparing GS-168 (15 mg, yield: 33.4%). MS (ESI) m/z=578.5 [M+H]⁺.

### Example 129. 2-(2,6-Dioxopiperidin-3-yl)-5-((3-(trans-3-(3-methyl-4-(quinoxalin-2-yl)-1H-pyrazol-1-yl)cyclobutyl)propyl)amino)isoindoline-1,3-dione (GS-171)

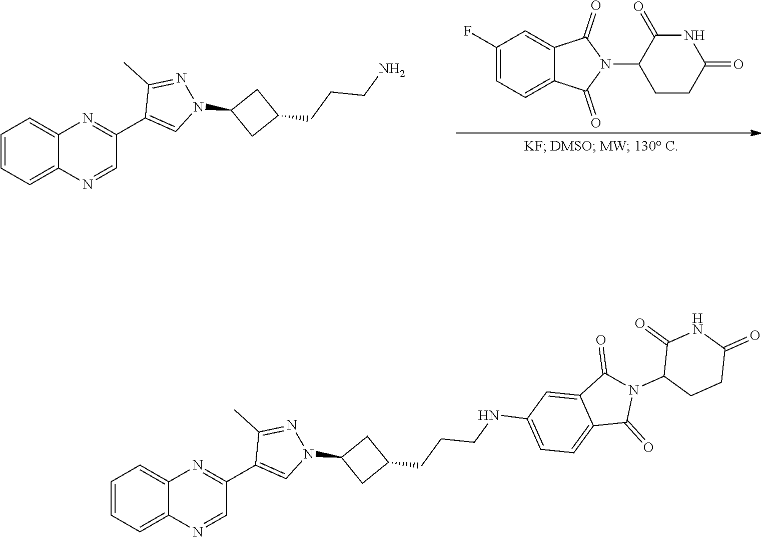 [SMILES: Cc1nn([C@H]2C[C@H](CCCN)C2)cc1-c1cnc2ccccc2n1 ; O=C1CCC(N2C(=O)c3ccc(F)cc3C2=O)C(=O)N1 ; Cc1nn([C@H]2C[C@H](CCCNc3ccc4c(c3)C(=O)N(C3CCC(=O)NC3=O)C4=O)C2)cc1-c1cnc2ccccc2n1]

GS-171 was synthesized following the similar procedure for preparing GS-168 (25 mg, yield: 23.2%). MS (ESI) m/z=578.5 [M+H]⁺.

### Example 130. 2-(2,6-Dioxopiperidin-3-yl)-5-((3-(3-(4-(7-(4-methylpiperazin-1-yl)quinoxalin-2-yl)-1H-pyrazol-1-yl)cyclobutyl)propyl)amino)isoindoline-1,3-dione (GS-172)

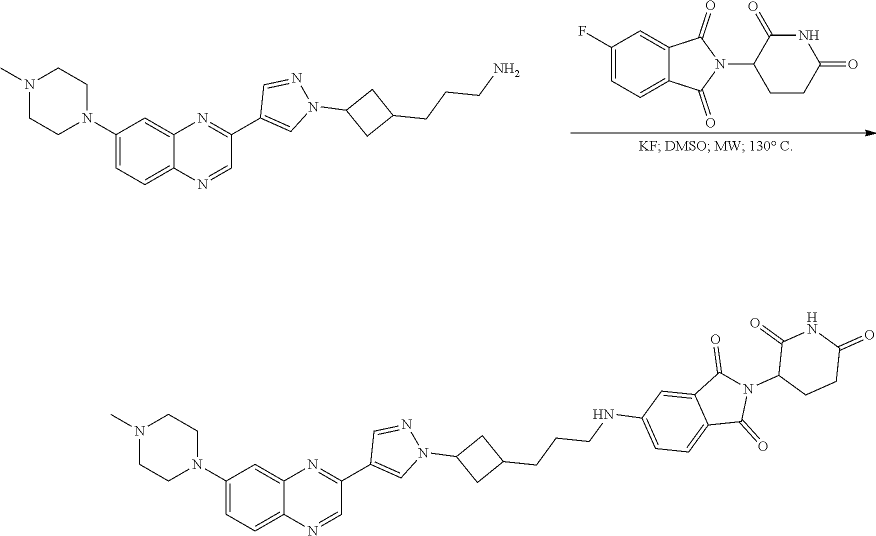 [SMILES: CN1CCN(c2ccc3ncc(-c4cnn(C5CC(CCCN)C5)c4)nc3c2)CC1 ; O=C1CCC(N2C(=O)c3ccc(F)cc3C2=O)C(=O)N1 ; CN1CCN(c2ccc3ncc(-c4cnn(C5CC(CCCNc6ccc7c(c6)C(=O)N(C6CCC(=O)NC6=O)C7=O)C5)c4)nc3c2)CC1]

GS-172 was synthesized following the similar procedure for preparing GS-107 (3.1 mg, yield: 9.5%). MS (ESI) m/z=662.5 [M+H]⁺.

### Example 131. 2-(2,6-Dioxopiperidin-3-yl)-5-((3-(3-(4-(7-morpholinoquinoxalin-2-yl)-1H-pyrazol-1-yl)cyclobutyl)propyl)amino)isoindoline-1,3-dione (GS-173)

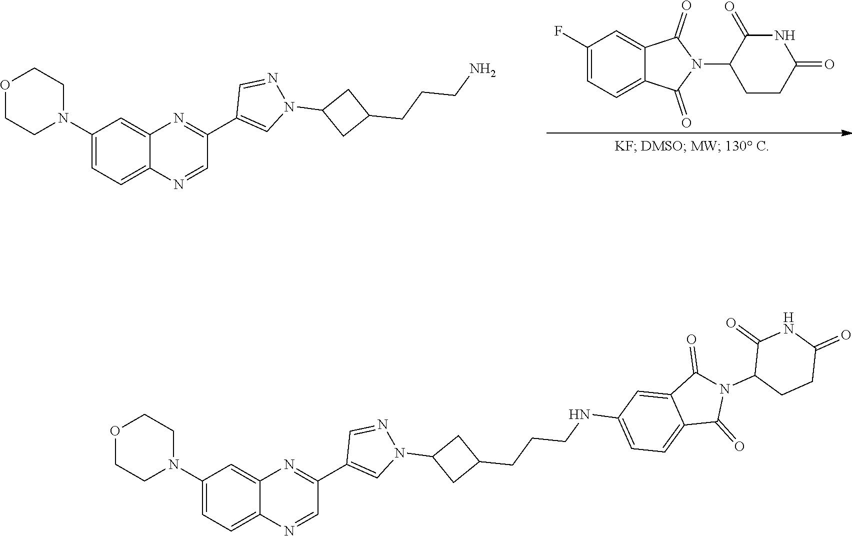 [SMILES: NCCCC1CC(n2cc(-c3cnc4ccc(N5CCOCC5)cc4n3)cn2)C1 ; O=C1CCC(N2C(=O)c3ccc(F)cc3C2=O)C(=O)N1 ; O=C1CCC(N2C(=O)c3ccc(NCCCC4CC(n5cc(-c6cnc7ccc(N8CCOCC8)cc7n6)cn5)C4)cc3C2=O)C(=O)N1]

GS-173 was synthesized following the similar procedure for preparing GS-107 (3.7 mg, yield: 7.5%). MS (ESI) m/z=649.5 [M+H]⁺.

### Example 132. 2-(2,6-Dioxopiperidin-3-yl)-5-((3-(3-(4-(7-(piperazin-1-yl)quinoxalin-2-yl)-1H-pyrazol-1-yl)cyclobutyl)propyl)amino)isoindoline-1,3-dione (GS-174)

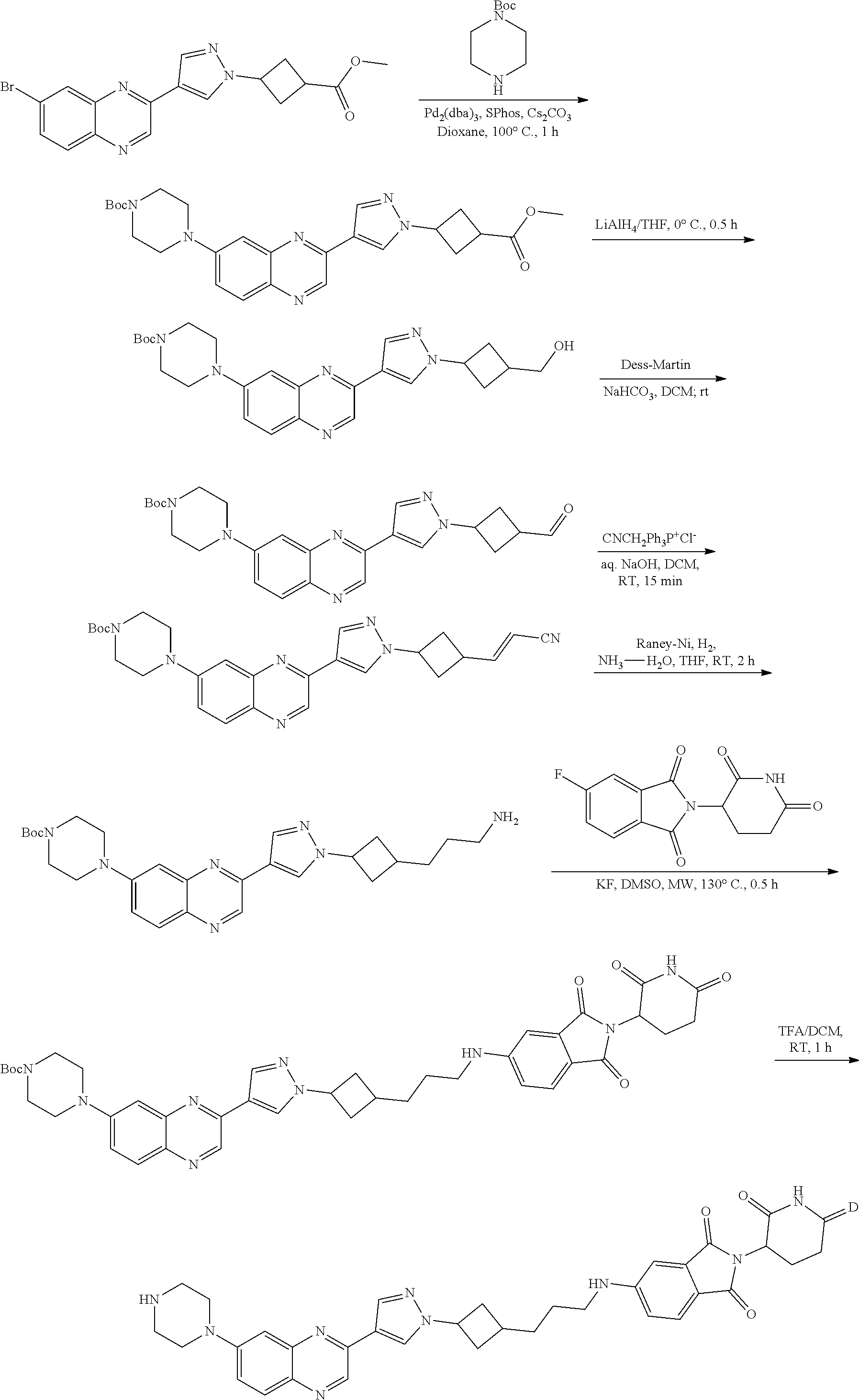

Step 1: Synthesis of tert-butyl 4-(3-(1-(3-(methoxycarbonyl)cyclobutyl)-1H-pyrazol-4-yl)quinoxalin-6-yl)piperazine-1-carboxylate

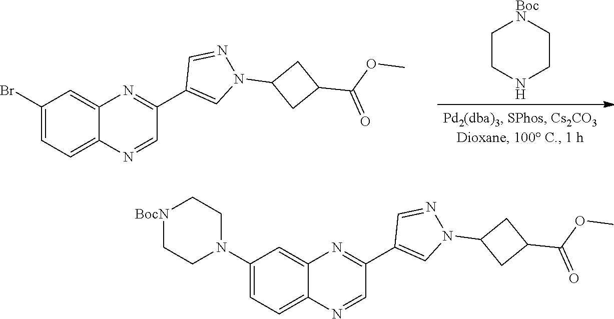 [SMILES: COC(=O)C1CC(n2cc(-c3cnc4ccc(Br)cc4n3)cn2)C1 ; CC(C)(C)OC(=O)N1CCNCC1 ; COC(=O)C1CC(n2cc(-c3cnc4ccc(N5CCN(C(=O)OC(C)(C)C)CC5)cc4n3)cn2)C1]

To a suspension of methyl 3-(4-(7-bromoquinoxalin-2-yl)-1H-pyrazol-1-yl)cyclobutane-1-carboxylate (200.0 mg, 0.52 mmol), tert-butyl piperazine-1-carboxylate (144.3 mg, 0.77 mmol), Cs₂CO₃ (420.9 mg, 1.29 mmol) and SPhos (64.1 mg, 0.16 mmol) in dioxane (5 mL) was added Pd₂(dba)₃ (71.1 mg, 0.08 mmol). After the mixture was stirred at 100° C. for 1 h, it was cooled to rt, diluted with EtOAc, and filtered through celite. The filtrate was concentrated, and The resulting residue was purified by reverse phase chromatography to provide the title compound (220 mg, 85.9% yield) as a pale white solid. MS (ESI) m/z=493.4 [M+H]⁺.

Step 2: Synthesis of tert-butyl 4-(3-(1-(3-(hydroxymethyl)cyclobutyl)-1H-pyrazol-4-yl)quinoxalin-6-yl)piperazine-1-carboxylate

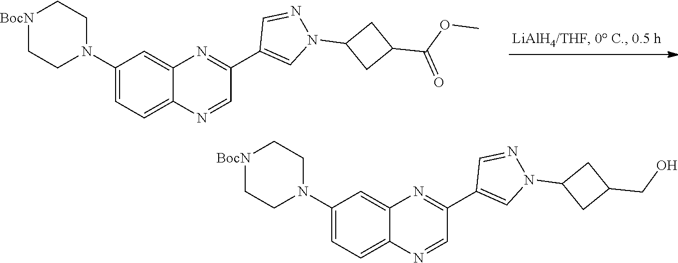 [SMILES: COC(=O)C1CC(n2cc(-c3cnc4ccc(N5CCN(C(=O)OC(C)(C)C)CC5)cc4n3)cn2)C1 ; CC(C)(C)OC(=O)N1CCN(c2ccc3ncc(-c4cnn(C5CC(CO)C5)c4)nc3c2)CC1]

To a solution of tert-butyl 4-(3-(1-(3-(methoxycarbonyl)cyclobutyl)-1H-pyrazol-4-yl) quinoxalin-6-yl)piperazine-1-carboxylate (220 mg, 0.45 mmol) in THF (5.0 mL) was added LAH (67.9 mg, 1.79 mmol) at 0° C. in portions over 5 min. The resulting mixture was stirred at the same temperature for 0.5 h, before it was quenched with solid Na₂SO₄ 10H₂O, and diluted with EtOAc. The suspension was filtered through celite. The filtrate was concentrated to provide the crude product (200 mg, 95.6% yield) which was used directly in the next step without further purification. MS (ESI) m/z=465.4 [M+H]⁺.

Step 3: Synthesis of tert-butyl 4-(3-(1-(3-formylcyclobutyl)-1H-pyrazol-4-yl)quinoxalin-6-yl)piperazine-1-carboxylate

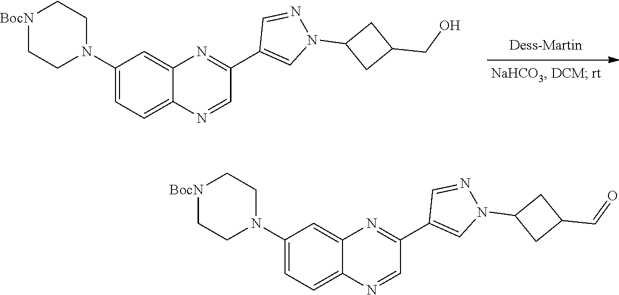 [SMILES: CC(C)(C)OC(=O)N1CCN(c2ccc3ncc(-c4cnn(C5CC(CO)C5)c4)nc3c2)CC1 ; CC(C)(C)OC(=O)N1CCN(c2ccc3ncc(-c4cnn(C5CC(C=O)C5)c4)nc3c2)CC1]

To a suspension of tert-butyl 4-(3-(1-(3-(hydroxymethyl)cyclobutyl)-1H-pyrazol-4-yl) quinoxalin-6-yl)piperazine-1-carboxylate (130 mg crude, 0.28 mmol) and NaHCO₃ (47.0 mg, 0.56 mmol) in DCM (5.0 mL) was added Dess-Martin Periodinane (178.1 mg, 0.42 mmol) at 0° C. in 2 portions. After it was stirred at the rt for 2 h, the reaction mixture was diluted with DCM and filtered through celite. The filtrate was concentrated to provide the crude product (120 mg, 92.6% yield) which was used directly in the next step without further purification. MS (ESI) m/z=463.3 [M+H]⁺.

Step 4: Synthesis of tert-butyl (E)-4-(3-(1-(3-(2-cyanovinyl)cyclobutyl)-1H-pyrazol-4-yl)quinoxalin-6-yl)piperazine-1-carboxylate

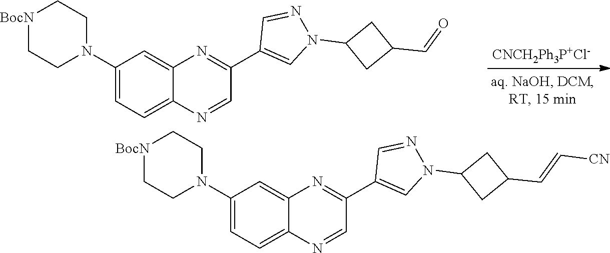

To a mixture of (cyanomethyl)triphenylphosphonium chloride (47.0 mg, 0.56 mmol) in DCM (2.0 mL) was added 30% of aqueous NaOH solution (0.2 mL) at 0° C. After the mixture was stirred for 5 min, tert-butyl 4-(3-(1-(3-formylcyclobutyl)-1H-pyrazol-4-yl)quinoxalin-6-yl) piperazine-1-carboxylate (120 mg crude, 0.28 mmol) was added. After the resulting mixture was stirred at the same temperature for another 10 min, it was diluted with ice water and extracted with DCM. The organic layer was dried over anhydrous Na₂SO₄ and concentrated. The resulting residue was purified by reverse phase chromatography to provide the title compound (80 mg, 36.7% yield for 3 steps) as a pale yellow solid. MS (ESI) m/z=486.4 [M+H]⁺.

Step 5: Synthesis of tert-butyl 4-(3-(1-(3-(3-aminopropyl)cyclobutyl)-1H-pyrazol-4-yl)quinoxalin-6-yl)piperazine-1-carboxylate

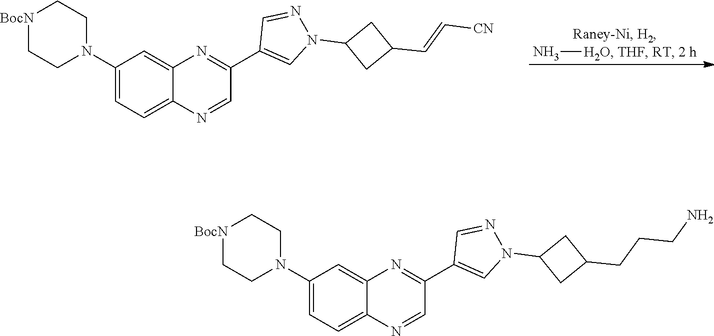

To a mixture of tert-butyl (E)-4-(3-(1-(3-(2-cyanovinyl)cyclobutyl)-1H-pyrazol-4-yl)quin oxalin-6-yl)piperazine-1-carboxylate (40.0 mg, 0.08 mmol) in THF (4.0 mL) and aqueous ammonia solution (0.2 mL) was added Raney-Ni catalyst (8.0 mg, 20% wt). After the mixture was stirred at rt under H₂ for 2 h, the reaction mixture was filtered through celite, and washed with EtOAc. The filtrate was concentrated to provide the crude product (25 mg crude, 63.6% yield) as a yellow solid which was used in the next step directly. MS (ESI) m/z=492.4 [M+H]⁺.

Step 6: Synthesis of tert-butyl 4-(3-(1-(3-(3-((2-(2,6-dioxopiperidin-3-yl)-1,3-dioxoisoindolin-5-yl)amino)propyl)cyclobutyl)-1H-pyrazol-4-yl)quinoxalin-6-yl)piperazine-1-carboxylate

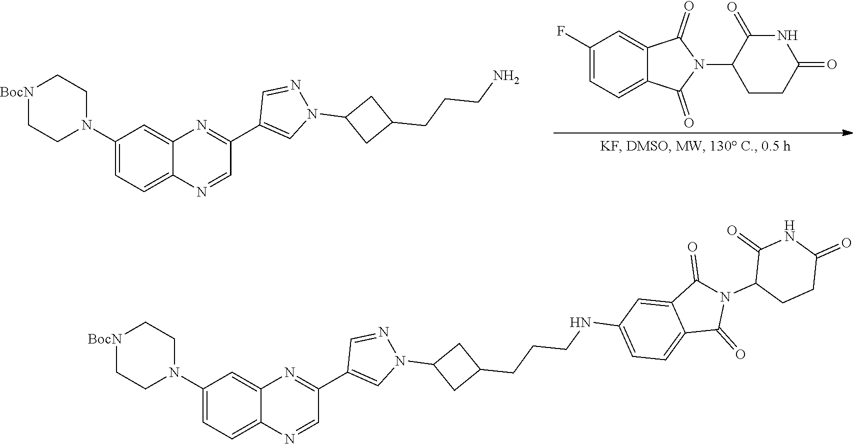 [SMILES: CC(C)(C)OC(=O)N1CCN(c2ccc3ncc(-c4cnn(C5CC(CCCN)C5)c4)nc3c2)CC1 ; O=C1CCC(N2C(=O)c3ccc(F)cc3C2=O)C(=O)N1 ; CC(C)(C)OC(=O)N1CCN(c2ccc3ncc(-c4cnn(C5CC(CCCNc6ccc7c(c6)C(=O)N(C6CCC(=O)NC6=O)C7=O)C5)c4)nc3c2)CC1]

To a mixture of tert-butyl 4-(3-(1-(3-(3-aminopropyl)cyclobutyl)-1H-pyrazol-4-yl) quinoxalin-6-yl)piperazine-1-carboxylate (25.0 mg crude, 0.05 mmol) and KF (29.0 mg, 0.50 mmol) in DMSO (0.5 mL) was added 2-(2,6-dioxopiperidin-3-yl)-5-fluoroisoindoline-1,3-dione (27.6 mg, 0.10 mmol). After the reaction was irradiated in a microwave tube at 130° C. for 0.5 h, the reaction mixture was cooled to rt and purified by reverse phase chromatography, followed by prep-TLC to provide the title compound (10 mg, 26.7% yield) as a yellow solid. MS (ESI) m/z=748.7 [M+H]⁺.

Step 7: Synthesis of 2-(2,6-dioxopiperidin-3-yl)-5-((3-(3-(4-(7-(piperazin-1-yl)quinoxalin-2-yl)-1H-pyrazol-1-yl)cyclobutyl)propyl)amino)isoindoline-1,3-dione

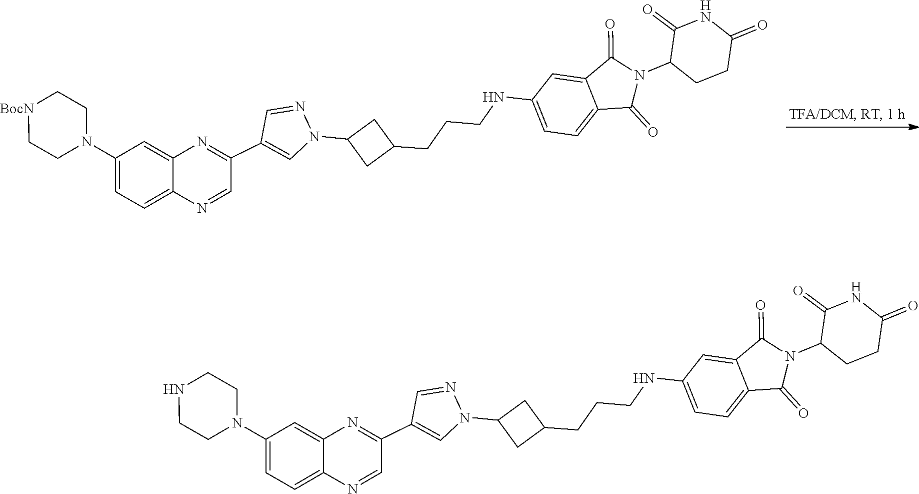 [SMILES: CC(C)(C)OC(=O)N1CCN(c2ccc3ncc(-c4cnn(C5CC(CCCNc6ccc7c(c6)C(=O)N(C6CCC(=O)NC6=O)C7=O)C5)c4)nc3c2)CC1 ; O=C1CCC(N2C(=O)c3ccc(NCCCC4CC(n5cc(-c6cnc7ccc(N8CCNCC8)cc7n6)cn5)C4)cc3C2=O)C(=O)N1]

To a solution of tert-butyl 4-(3-(1-(3-(3-((2-(2,6-dioxopiperidin-3-yl)-1,3-dioxoisoindolin-5-yl)amino)propyl)cyclobutyl)-1H-pyrazol-4-yl)quinoxalin-6-yl)piperazine-1-carboxylate (10 mg, 0.013 mmol) in DCM (0.5 mL) was added TFA (0.3 mL). After the reaction mixture was stirred at rt for 1 h, it was concentrated in vacuum to provide a residue. The crude product was purified by prep-HPLC to provide the title compound (3.1 mg, 34.8% yield) as a red brown solid. MS (ESI) m/z=648.6 [M+H]⁺.

### Example 133. 3-(1-(3-(3-((2-(2,6-Dioxopiperidin-3-yl)-1,3-dioxoisoindolin-5-yl)amino)propyl)cyclobutyl)-1H-pyrazol-4-yl)quinoxaline-6-carbonitrile (GS-175)

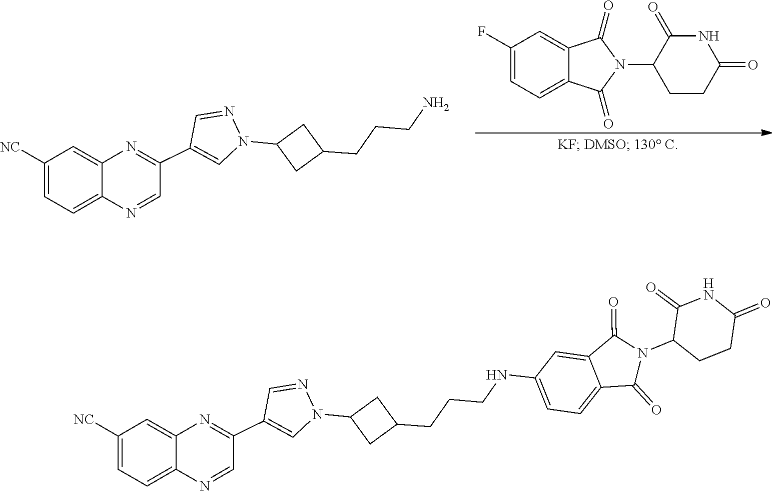 [SMILES: N#Cc1ccc2ncc(-c3cnn(C4CC(CCCN)C4)c3)nc2c1 ; O=C1CCC(N2C(=O)c3ccc(F)cc3C2=O)C(=O)N1 ; N#Cc1ccc2ncc(-c3cnn(C4CC(CCCNc5ccc6c(c5)C(=O)N(C5CCC(=O)NC5=O)C6=O)C4)c3)nc2c1]

GS-175 was synthesized following the similar procedure for preparing GS-107 (1.3 mg, yield: 7.9%). MS (ESI) m/z=589.5 [M+H]⁺.

### Example 134. 2-(2,6-Dioxopiperidin-3-yl)-5-((3-(trans-3-(4-(quinoxalin-2-yl)-1H-pyrazol-1-yl)cyclobutyl)propyl)amino)isoindoline-1,3-dione (GS-176)

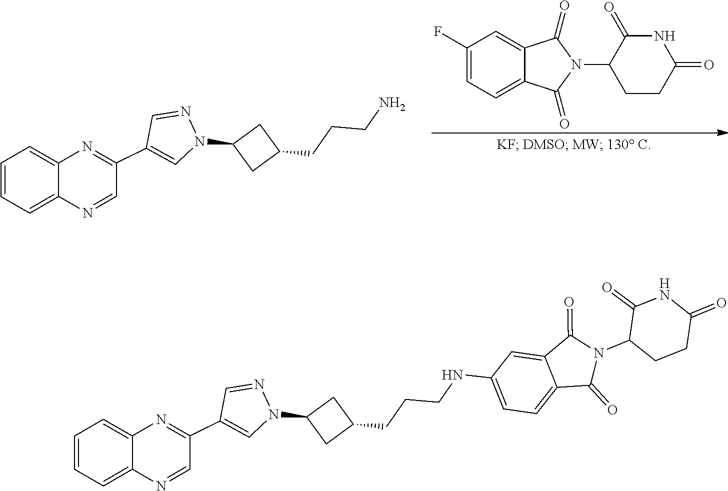 [SMILES: NCCC[C@H]1C[C@H](n2cc(-c3cnc4ccccc4n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccccc7n6)cn5)C4)cc3C2=O)C(=O)N1]

GS-176 was synthesized following the similar procedure for preparing GS-168 (35 mg, yield: 12.7%). MS (ESI) m/z=564.4 [M+H]⁺.

### Example 135. 2-(2,6-Dioxopiperidin-3-yl)-5-((3-(cis-3-(4-(quinoxalin-2-yl)-1H-pyrazol-1-yl)cyclobutyl)propyl)amino)isoindoline-1,3-dione (GS-177)

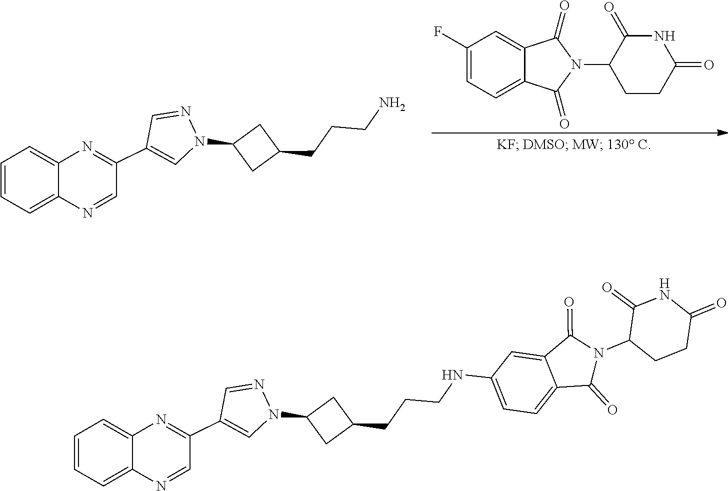 [SMILES: NCCC[C@H]1C[C@@H](n2cc(-c3cnc4ccccc4n3)cn2)C1 ; O=C1CCC(N2C(=O)c3ccc(F)cc3C2=O)C(=O)N1 ; O=C1CCC(N2C(=O)c3ccc(NCCC[C@H]4C[C@@H](n5cc(-c6cnc7ccccc7n6)cn5)C4)cc3C2=O)C(=O)N1]

GS-177 was synthesized following the similar procedure for preparing GS-168 (36 mg, yield: 15.2%). MS (ESI) m/z=564.4 [M+H]⁺.

### Example 136. 2-(2,6-Dioxopiperidin-3-yl)-5-(((trans-3-(4-(quinoxalin-2-yl)-1H-pyrazol-1-yl)cyclobutyl)methyl)amino)isoindoline-1,3-dione (GS-178)

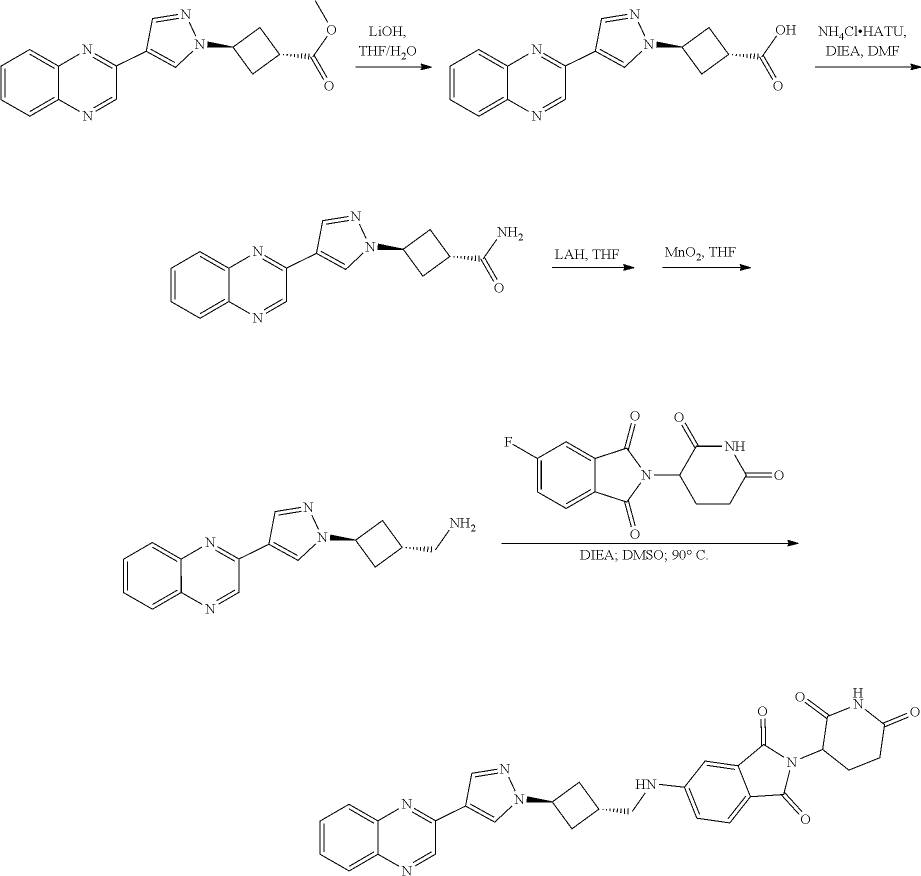 [SMILES: COC(=O)[C@H]1C[C@H](n2cc(-c3cnc4ccccc4n3)cn2)C1 ; O=C(O)[C@H]1C[C@H](n2cc(-c3cnc4ccccc4n3)cn2)C1 ; NC(=O)[C@H]1C[C@H](n2cc(-c3cnc4ccccc4n3)cn2)C1 ; NC[C@H]1C[C@H](n2cc(-c3cnc4ccccc4n3)cn2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cnc7ccccc7n6)cn5)C4)cc3C2=O)C(=O)N1]

Step 1: Synthesis of trans-3-(4-(quinoxalin-2-yl)-1H-pyrazol-1-yl)cyclobutane-1-carboxylic acid

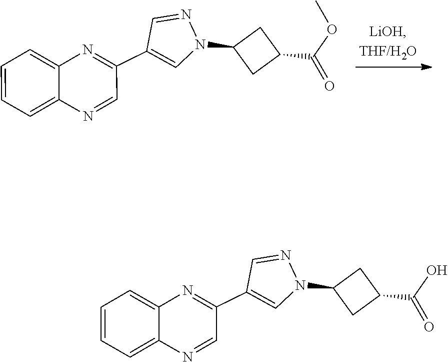 [SMILES: COC(=O)[C@H]1C[C@H](n2cc(-c3cnc4ccccc4n3)cn2)C1 ; O=C(O)[C@H]1C[C@H](n2cc(-c3cnc4ccccc4n3)cn2)C1]

To a solution of methyl trans-3-(4-(quinoxalin-2-yl)-1H-pyrazol-1-yl)cyclobutane-1-carboxylate (358 mg, 1.16 mmol) in THF (5.0 mL) and H₂O (5.0 mL) was added LiOHH₂O (293 mg, 7.0 mmol). The reaction was stirred at rt for 1 h, before 6 N HCl was added to adjust pH to 2˜3. The mixture was concentrated to provide the crude title compound (340 mg, 99.6% yield) which was used directly in the next step without further purification. MS (ESI) m/z=295.1 [M+H]⁺.

Step 2: Synthesis of trans-3-(4-(quinoxalin-2-yl)-1H-pyrazol-1-yl)cyclobutane-1-carboxamide

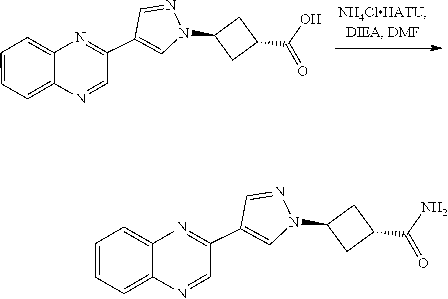 [SMILES: O=C(O)[C@H]1C[C@H](n2cc(-c3cnc4ccccc4n3)cn2)C1 ; NC(=O)[C@H]1C[C@H](n2cc(-c3cnc4ccccc4n3)cn2)C1]

To a solution of trans-3-(4-(quinoxalin-2-yl)-1H-pyrazol-1-yl)cyclobutane-1-carboxylic acid (340 mg, 1.16 mmol) in DMF (5.0 mL) was added DIEA (1.3 mL, 5.81 mmol), HATU (662.5 mg, 1.74 mmol) and NH₄Cl (70 mg, 1.28 mmol). The reaction mixture was stirred at rt for 1 h, before it was purified by reverse column chromatography to provide the title compound (140 mg, 41% yield) as a yellow solid. MS (ESI) m/z=294.1 [M+H]⁺.

Step 3: Synthesis of (trans-3-(4-(quinoxalin-2-yl)-1H-pyrazol-1-yl)cyclobutyl)methanamine

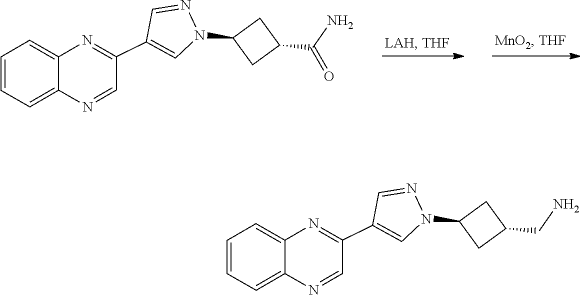 [SMILES: NC(=O)[C@H]1C[C@H](n2cc(-c3cnc4ccccc4n3)cn2)C1 ; NC[C@H]1C[C@H](n2cc(-c3cnc4ccccc4n3)cn2)C1]

To a solution of trans-3-(4-(quinoxalin-2-yl)-1H-pyrazol-1-yl)cyclobutane-1-carboxamide (140 mg, 0.47 mmol) in THF (7.0 mL) was added LiAlH₄ (30 mg, 0.79 mmol) at 0° C. The mixture was stirred at 70° C. for 1 h and then cooled to rt. The reaction was quenched with H₂O (1.0 mL) and stirred at rt for 5.0 min, then MnO₂ (383 mg, 4.4 mmol) was added and stirred at rt for another 30 min. The reaction mixture was filtered and purified by reverse column chromatography to provide the title compound (132.1 mg, 99% yield) as a yellow solid. MS (ESI) m/z=280.1 [M+H]⁺.

Step 4: Synthesis of 2-(2,6-dioxopiperidin-3-yl)-5-(((trans-3-(4-(quinoxalin-2-yl)-1H-pyrazol-1-yl)cyclobutyl)methyl)amino)isoindoline-1,3-dione

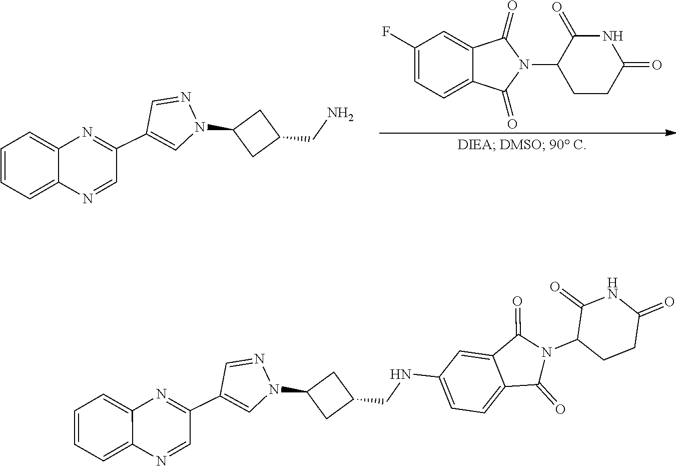 [SMILES: NC[C@H]1C[C@H](n2cc(-c3cnc4ccccc4n3)cn2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cnc7ccccc7n6)cn5)C4)cc3C2=O)C(=O)N1]

To a solution of (trans-3-(4-(quinoxalin-2-yl)-1H-pyrazol-1-yl)cyclobutyl)methanamine (132.1 mg, 0.473 mmol) in DMSO (2.0 mL) was added 2-(2,6-dioxopiperidin-3-yl)-5-fluoroisoindoline-1,3-dione (157 mg, 0.568 mmol) and DIEA (0.83 mL, 4.73 mmol). The reaction was stirred at 90° C. for 16 h, then it was cooled to rt and purified by reverse column chromatography to provide the title compound (28.7 mg, 11.3% yield) as a yellow solid. MS (ESI) m/z=536.3 [M+H]⁺.

### Example 137. 2-(2,6-Dioxopiperidin-3-yl)-5-(((cis-3-(4-(quinoxalin-2-yl)-1H-pyrazol-1-yl)cyclobutyl)methyl)amino)isoindoline-1,3-dione (GS-179)

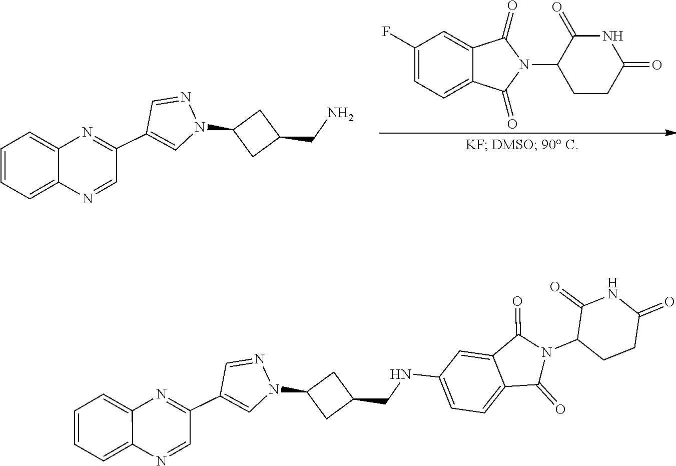 [SMILES: NC[C@H]1C[C@@H](n2cc(-c3cnc4ccccc4n3)cn2)C1 ; O=C1CCC(N2C(=O)c3ccc(F)cc3C2=O)C(=O)N1 ; O=C1CCC(N2C(=O)c3ccc(NC[C@H]4C[C@@H](n5cc(-c6cnc7ccccc7n6)cn5)C4)cc3C2=O)C(=O)N1]

GS-179 was synthesized following the similar procedure for preparing GS-178 (35.3 mg, yield: 14.6%). MS (ESI) m/z=536.3 [M+H]⁺.

### Example 138. 2-(2,6-Dioxopiperidin-3-yl)-5-(((trans-3-(4-(7-(tetrahydro-2H-pyran-4-yl)quinoxalin-2-yl)-1H-pyrazol-1-yl)cyclobutyl)methyl)amino)isoindoline-1,3-dione (GS-180)

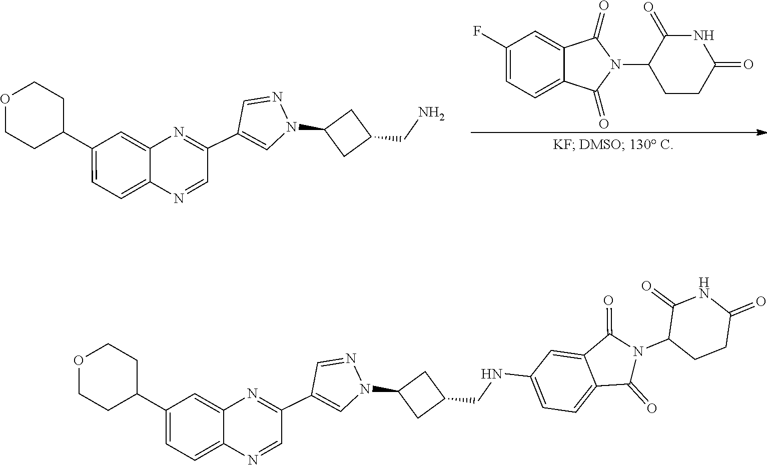 [SMILES: NC[C@H]1C[C@H](n2cc(-c3cnc4ccc(C5CCOCC5)cc4n3)cn2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cnc7ccc(C8CCOCC8)cc7n6)cn5)C4)cc3C2=O)C(=O)N1]

GS-180 was synthesized following the similar procedure for preparing GS-178 (14 mg, yield: 11.8%). MS (ESI) m/z=620.5 [M+H]⁺.

### Example 139. 2-(2,6-Dioxopiperidin-3-yl)-5-((3-(trans-3-(4-(7-(tetrahydro-2H-pyran-4-yl)quinoxalin-2-yl)-1H-pyrazol-1-yl)cyclobutyl)propyl)amino)isoindoline-1,3-dione (GS-181)

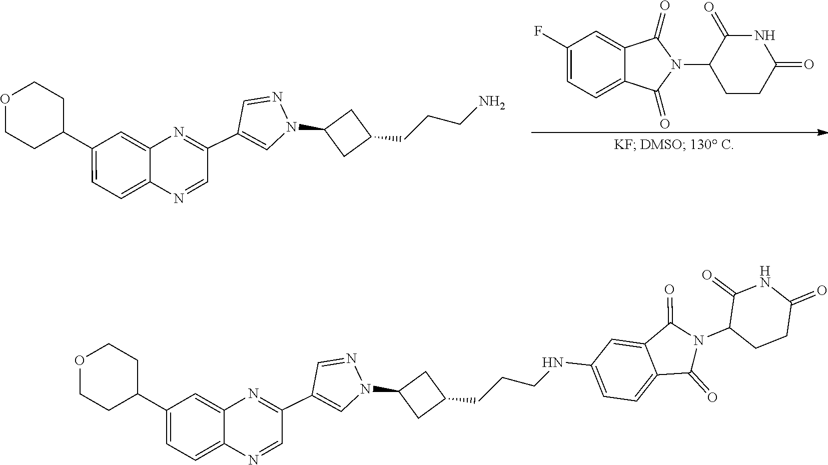 [SMILES: NCCC[C@H]1C[C@H](n2cc(-c3cnc4ccc(C5CCOCC5)cc4n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccc(C8CCOCC8)cc7n6)cn5)C4)cc3C2=O)C(=O)N1]

GS-181 was synthesized following the similar procedure for preparing GS-168 (20 mg, yield: 30%). MS (ESI) m/z=648.6 [M+H]⁺.

### Example 140. 2-(2,6-Dioxopiperidin-3-yl)-5-((3-(cis-3-(4-(7-(tetrahydro-2H-pyran-4-yl)quinoxalin-2-yl)-1H-pyrazol-1-yl)cyclobutyl)propyl)amino)isoindoline-1,3-dione (GS-182)

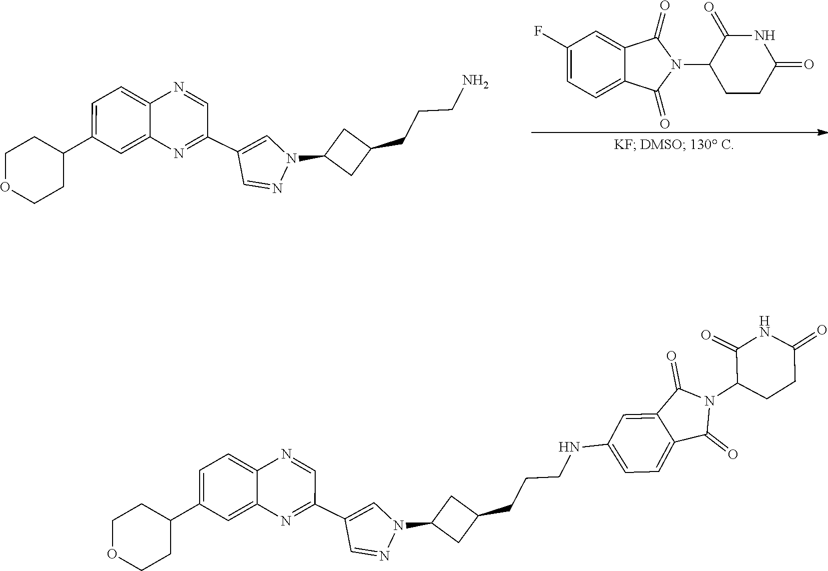 [SMILES: NCCC[C@H]1C[C@@H](n2cc(-c3cnc4ccc(C5CCOCC5)cc4n3)cn2)C1 ; O=C1CCC(N2C(=O)c3ccc(F)cc3C2=O)C(=O)N1 ; O=C1CCC(N2C(=O)c3ccc(NCCC[C@H]4C[C@@H](n5cc(-c6cnc7ccc(C8CCOCC8)cc7n6)cn5)C4)cc3C2=O)C(=O)N1]

GS-182 was synthesized following the similar procedure for preparing GS-168 (24 mg, yield: 22.3%). MS (ESI) m/z=648.6 [M+H]⁺.

### Example 141. 2-(2,6-Dioxopiperidin-3-yl)-5-((4-(2-(4-(quinoxalin-2-yl)-1H-pyrazol-1-yl)ethyl)cyclohexyl)amino)isoindoline-1,3-dione (GS-183)

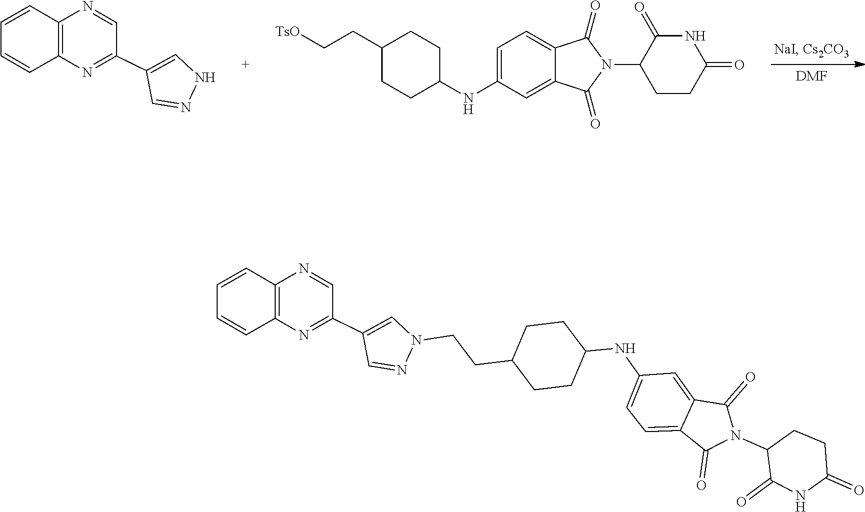

GS-183 was synthesized following the similar procedure for preparing GS-105 (4.6 mg, yield: 14.7%). MS (ESI) m/z=578.5 [M+H]⁺.

### Example 142. 2-(2,6-Dioxopiperidin-3-yl)-5-((4-(3-(4-(quinoxalin-2-yl)-1H-pyrazol-1-yl)propyl)cyclohexyl)amino)isoindoline-1,3-dione (GS-184)

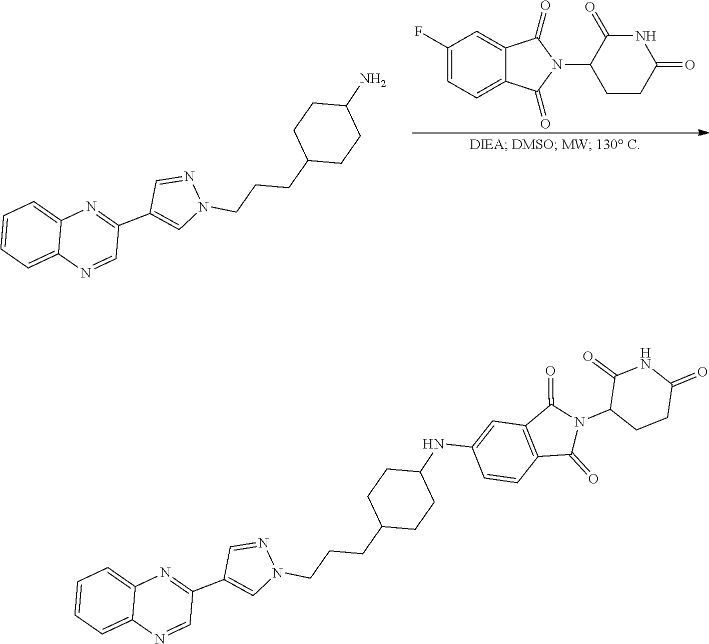 [SMILES: NC1CCC(CCCn2cc(-c3cnc4ccccc4n3)cn2)CC1 ; O=C1CCC(N2C(=O)c3ccc(F)cc3C2=O)C(=O)N1 ; O=C1CCC(N2C(=O)c3ccc(NC4CCC(CCCn5cc(-c6cnc7ccccc7n6)cn5)CC4)cc3C2=O)C(=O)N1]

GS-184 was synthesized following the similar procedure for preparing GS-107 (60 mg, yield: 20.7%). MS (ESI) m/z=592.5 [M+H]⁺.

### Example 143. 2-(2,6-Dioxopiperidin-3-yl)-5-(((trans-3-(4-(7-(4-methylpiperazin-1-yl)quinoxalin-2-yl)-1H-pyrazol-1-yl)cyclobutyl)methyl)amino)isoindoline-1,3-dione (GS-185)

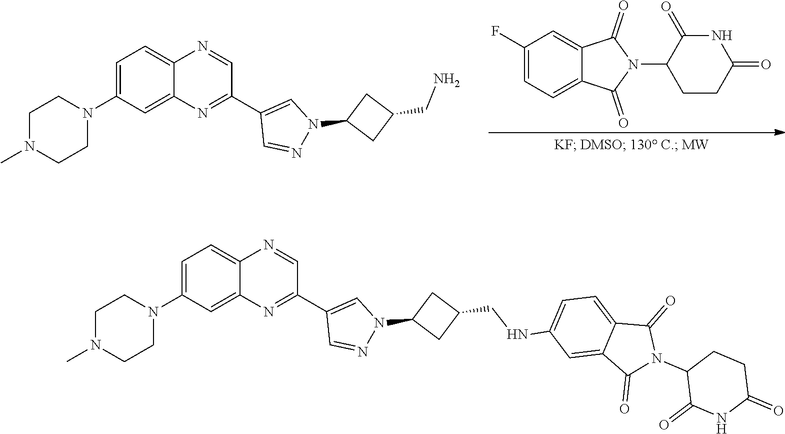 [SMILES: CN1CCN(c2ccc3ncc(-c4cnn([C@H]5C[C@H](CN)C5)c4)nc3c2)CC1 ; O=C1CCC(N2C(=O)c3ccc(F)cc3C2=O)C(=O)N1 ; CN1CCN(c2ccc3ncc(-c4cnn([C@H]5C[C@H](CNc6ccc7c(c6)C(=O)N(C6CCC(=O)NC6=O)C7=O)C5)c4)nc3c2)CC1]

GS-185 was synthesized following the similar procedure for preparing GS-178 (13 mg, yield: 22.8%). MS (ESI) m/z=634.5 [M+H]⁺.

### Example 144. 2-(2,6-Dioxopiperidin-3-yl)-5-(((cis-3-(4-(7-(piperazin-1-yl)quinoxalin-2-yl)-1H-pyrazol-1-yl)cyclobutyl)methyl)amino)isoindoline-1,3-dione (GS-186)

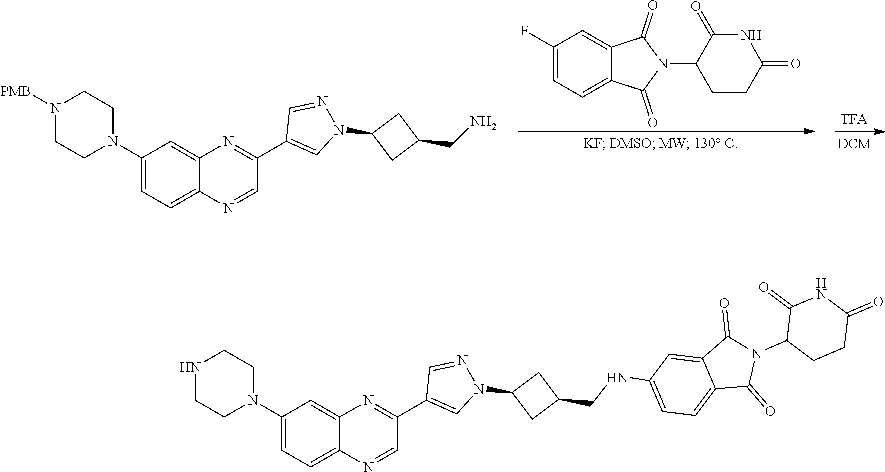

GS-186 was synthesized following the similar procedure for preparing GS-174 (30 mg, yield: 11.5%). MS (ESI) m/z=620.5 [M+H]⁺.

### Example 145. 2-(2,6-Dioxopiperidin-3-yl)-5-(((trans-3-(4-(7-(piperazin-1-yl)quinoxalin-2-yl)-1H-pyrazol-1-yl)cyclobutyl)methyl)amino)isoindoline-1,3-dione (GS-187)

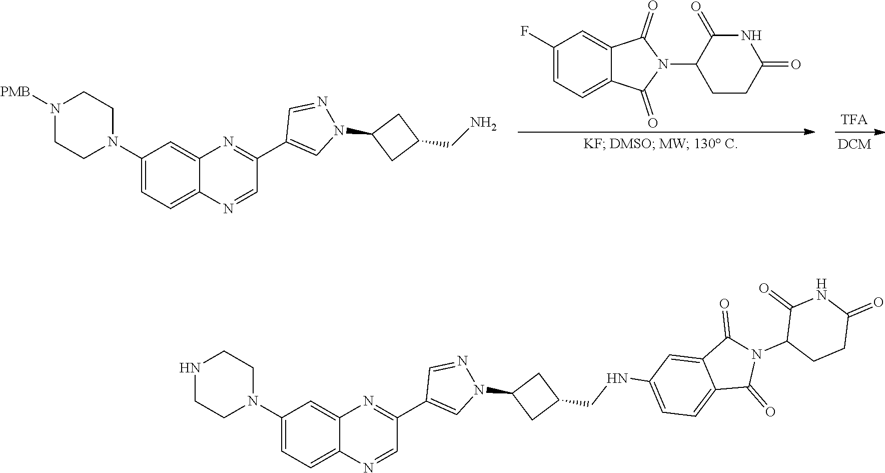

GS-187 was synthesized following the similar procedure for preparing GS-174 (4.2 mg, yield: 4.7%). MS (ESI) m/z=620.5 [M+H]⁺.

### Example 146. 2-(2,6-Dioxopiperidin-3-yl)-5-((3-(trans-3-(4-(7-morpholinoquinoxalin-2-yl)-1H-pyrazol-1-yl)cyclobutyl)propyl)amino)isoindoline-1,3-dione (GS-188)

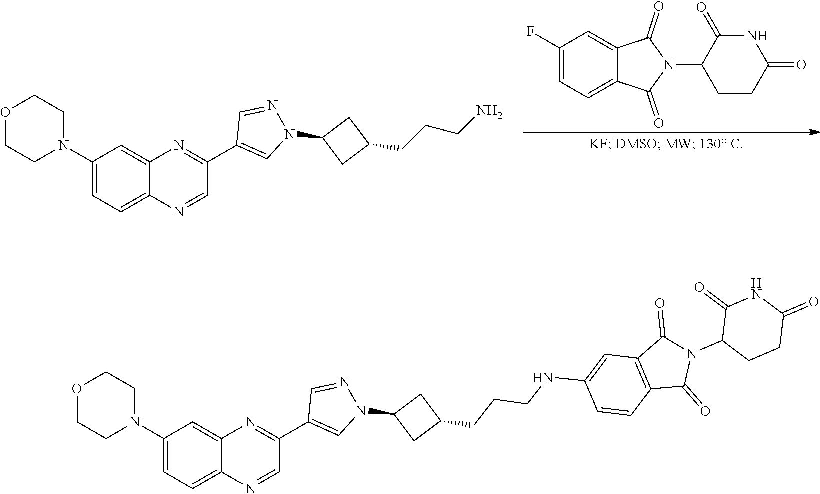 [SMILES: NCCC[C@H]1C[C@H](n2cc(-c3cnc4ccc(N5CCOCC5)cc4n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccc(N8CCOCC8)cc7n6)cn5)C4)cc3C2=O)C(=O)N1]

GS-188 was synthesized following the similar procedure for preparing GS-168 (4.1 mg, yield: 4%). MS (ESI) m/z=649.6 [M+H]⁺.

### Example 147. 2-(2,6-Dioxopiperidin-3-yl)-5-(((cis-3-(4-(7-morpholinoquinoxalin-2-yl)-1H-pyrazol-1-yl)cyclobutyl)methyl)amino)isoindoline-1,3-dione (GS-189)

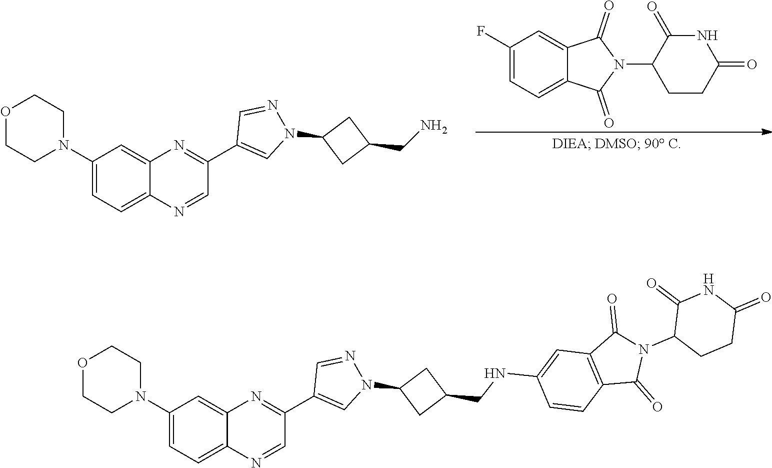 [SMILES: NC[C@H]1C[C@@H](n2cc(-c3cnc4ccc(N5CCOCC5)cc4n3)cn2)C1 ; O=C1CCC(N2C(=O)c3ccc(F)cc3C2=O)C(=O)N1 ; O=C1CCC(N2C(=O)c3ccc(NC[C@H]4C[C@@H](n5cc(-c6cnc7ccc(N8CCOCC8)cc7n6)cn5)C4)cc3C2=O)C(=O)N1]

GS-189 was synthesized following the similar procedure for preparing GS-178 (8 mg, yield: 4.7%). MS (ESI) m/z=621.5 [M+H]⁺.

### Example 148. 2-(2,6-Dioxopiperidin-3-yl)-5-(((cis-3-(4-(7-(4-methylpiperazin-1-yl)quinoxalin-2-yl)-1H-pyrazol-1-yl)cyclobutyl)methyl)amino)isoindoline-1,3-dione (GS-190)

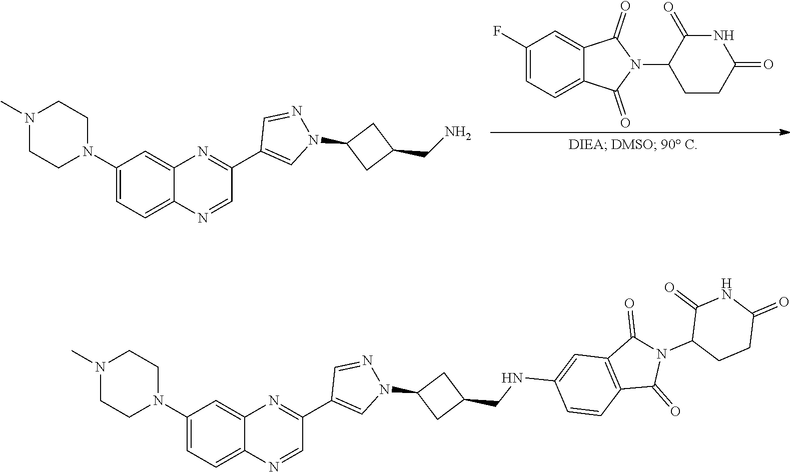 [SMILES: CN1CCN(c2ccc3ncc(-c4cnn([C@H]5C[C@@H](CN)C5)c4)nc3c2)CC1 ; O=C1CCC(N2C(=O)c3ccc(F)cc3C2=O)C(=O)N1 ; CN1CCN(c2ccc3ncc(-c4cnn([C@H]5C[C@@H](CNc6ccc7c(c6)C(=O)N(C6CCC(=O)NC6=O)C7=O)C5)c4)nc3c2)CC1]

GS-190 was synthesized following the similar procedure for preparing GS-178 (18 mg, yield: 9.7%). MS (ESI) m/z=634.6 [M+H]⁺.

### Example 149. 2-(2,6-Dioxopiperidin-3-yl)-5-(((trans-3-(4-(7-morpholinoquinoxalin-2-yl)-1H-pyrazol-1-yl)cyclobutyl)methyl)amino)isoindoline-1,3-dione (GS-191)

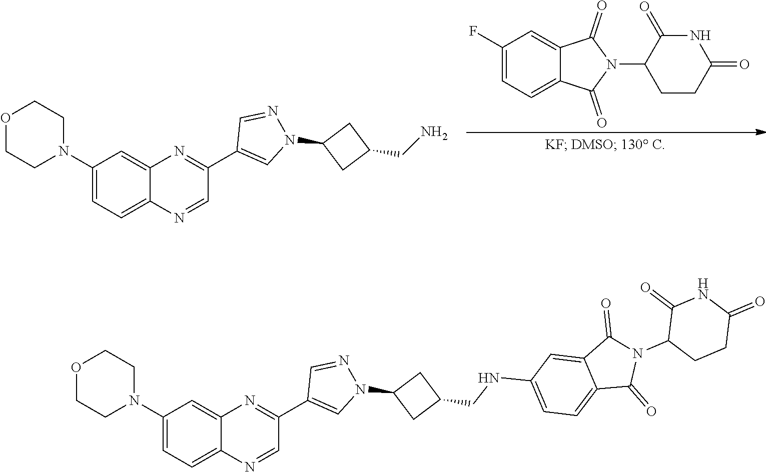 [SMILES: NC[C@H]1C[C@H](n2cc(-c3cnc4ccc(N5CCOCC5)cc4n3)cn2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cnc7ccc(N8CCOCC8)cc7n6)cn5)C4)cc3C2=O)C(=O)N1]

GS-191 was synthesized following the similar procedure or preparing GS-178 (35 mg, yield: 22.6%). MS (ESI) m/z=621.5 [M+H]⁺.

### Example 150. 2-(2,6-Dioxopiperidin-3-yl)-5-(((3-(4-(4-(quinoxalin-2-yl)-1H-pyrazol-1-yl)piperidine-1-carbonyl)cyclobutyl)methyl)amino)isoindoline-1,3-dione (GS-192)

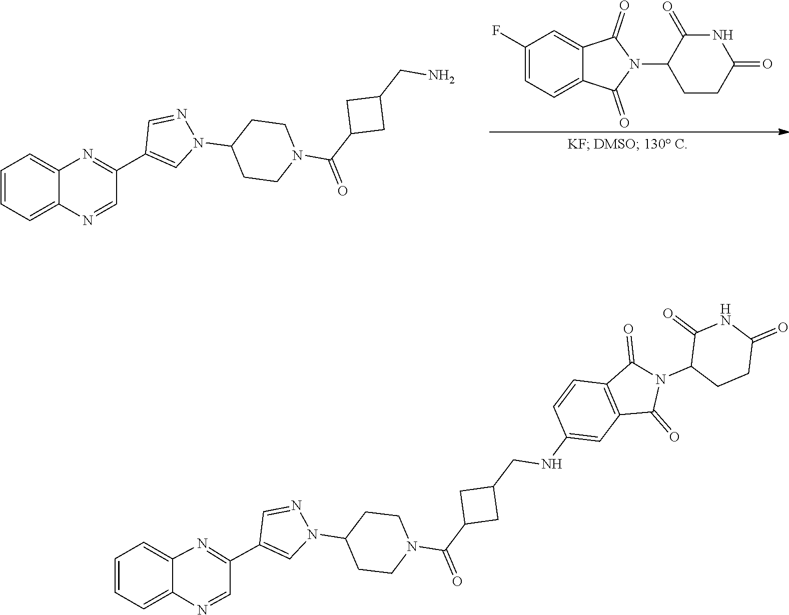 [SMILES: NCC1CC(C(=O)N2CCC(n3cc(-c4cnc5ccccc5n4)cn3)CC2)C1 ; O=C1CCC(N2C(=O)c3ccc(F)cc3C2=O)C(=O)N1 ; O=C1CCC(N2C(=O)c3ccc(NCC4CC(C(=O)N5CCC(n6cc(-c7cnc8ccccc8n7)cn6)CC5)C4)cc3C2=O)C(=O)N1]

GS-192 was synthesized following the similar procedure for preparing GS-107 (92 mg, yield: 34.3%). MS (ESI) m/z=647.5 [M+H]⁺.

### Example 151. 2-(2,6-Dioxopiperidin-3-yl)-5-(((cis-3-(4-(7-(tetrahydro-2H-pyran-4-yl)quinoxalin-2-yl)-1H-pyrazol-1-yl)cyclobutyl)methyl)amino)isoindoline-1,3-dione (GS-193)

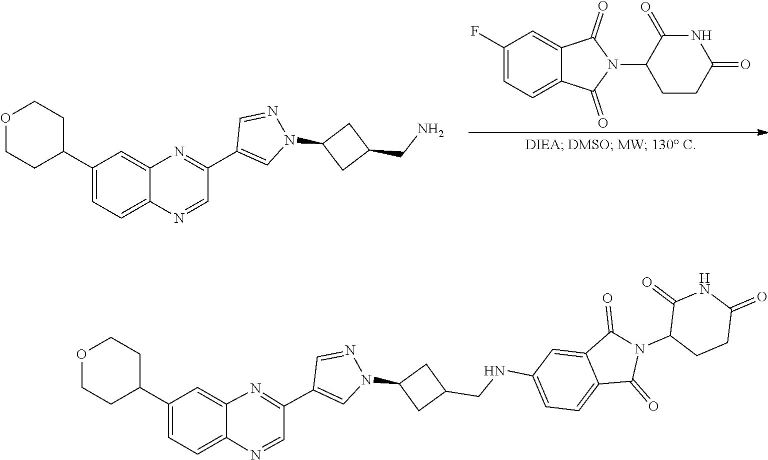

GS-193 was synthesized following the similar procedure for preparing GS-178 (16.3 mg, yield: 19%). MS (ESI) m/z=620.5 [M+H]⁺.

### Example 152. 2-(2,6-Dioxopiperidin-3-yl)-5-((2-(3-(4-(4-(quinoxalin-2-yl)-1H-pyrazol-1-yl)piperidine-1-carbonyl)bicyclo[1.1.1]pentan-1-yl)ethyl)amino)isoindoline-1,3-dione (GS-194)

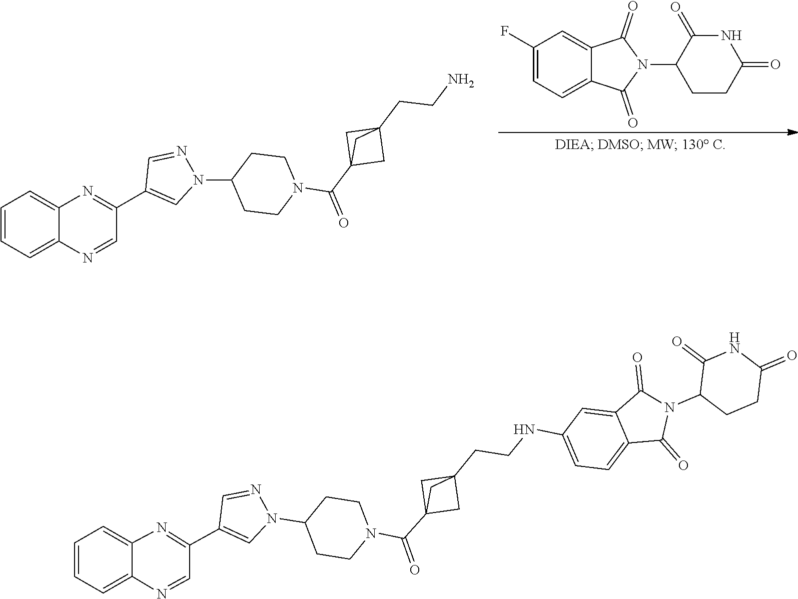 [SMILES: NCCC12CC(C(=O)N3CCC(n4cc(-c5cnc6ccccc6n5)cn4)CC3)(C1)C2 ; O=C1CCC(N2C(=O)c3ccc(F)cc3C2=O)C(=O)N1 ; O=C1CCC(N2C(=O)c3ccc(NCCC45CC(C(=O)N6CCC(n7cc(-c8cnc9ccccc9n8)cn7)CC6)(C4)C5)cc3C2=O)C(=O)N1]

GS-194 was synthesized following the similar procedure for preparing GS-107 (34 mg, yield: 15.2%). MS (ESI) m/z=673.5 [M+H]⁺.

### Example 153. 2-(2,6-Dioxopiperidin-3-yl)-5-((3-(trans-3-(4-(7-(piperazin-1-yl)quinoxalin-2-yl)-1H-pyrazol-1-yl)cyclobutyl)propyl)amino)isoindoline-1,3-dione (GS-195)

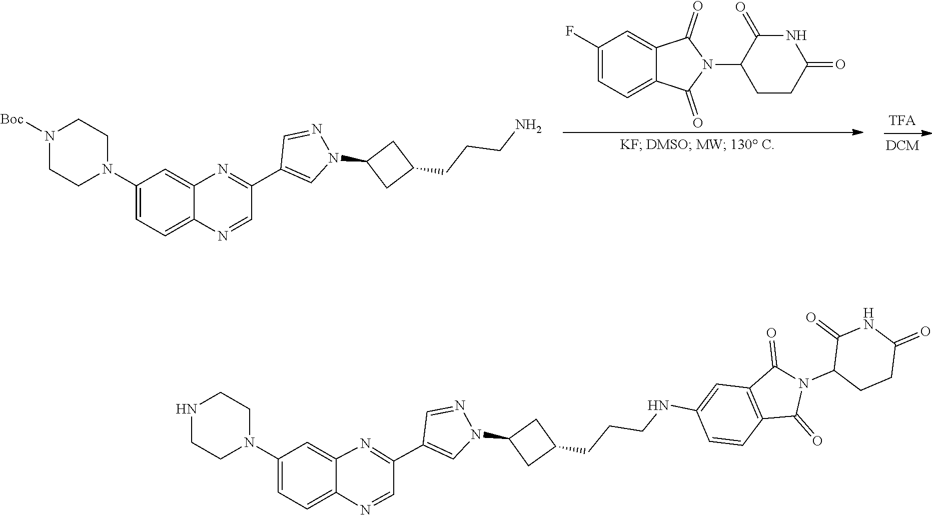 [SMILES: CC(C)(C)OC(=O)N1CCN(c2ccc3ncc(-c4cnn([C@H]5C[C@H](CCCN)C5)c4)nc3c2)CC1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccc(N8CCNCC8)cc7n6)cn5)C4)cc3C2=O)C(=O)N1]

GS-195 was synthesized following the similar procedure for preparing GS-174 (40 mg, yield: 76%). MS (ESI) m/z=648.6 [M+H]⁺.

### Example 154. 2-(2,6-Dioxopiperidin-3-yl)-5-((3-(trans-3-(4-(7-(piperidin-4-ylamino)quinoxalin-2-yl)-1H-pyrazol-1-yl)cyclobutyl)propyl)amino)isoindoline-1,3-dione (GS-196)

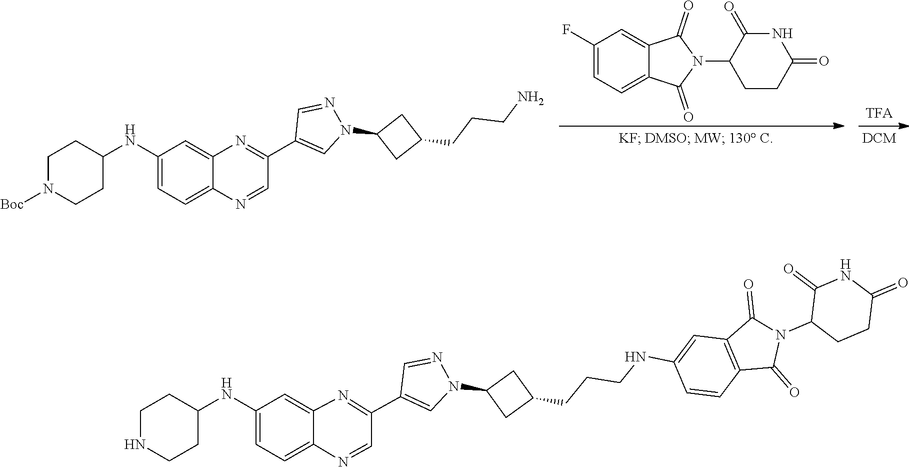 [SMILES: CC(C)(C)OC(=O)N1CCC(Nc2ccc3ncc(-c4cnn([C@H]5C[C@H](CCCN)C5)c4)nc3c2)CC1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccc(NC8CCNCC8)cc7n6)cn5)C4)cc3C2=O)C(=O)N1]

GS-196 was synthesized following the similar procedure for preparing GS-174 (11.3 mg, yield: 21.6%). MS (ESI) m/z=662.6 [M+H]⁺.

### Example 155. 3-(1-(trans-3-(3-((2-(2,6-Dioxopiperidin-3-yl)-1,3-dioxoisoindolin-5-yl)amino)propyl)cyclobutyl)-1H-pyrazol-4-yl)quinoxaline-5-carbonitrile (GS-197)

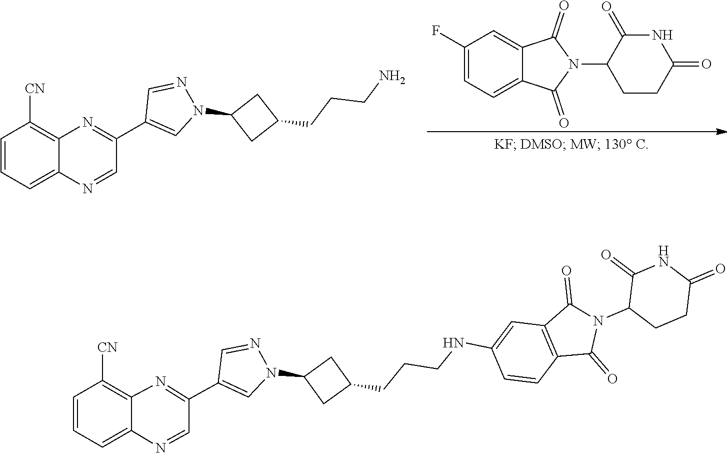 [SMILES: N#Cc1cccc2ncc(-c3cnn([C@H]4C[C@H](CCCN)C4)c3)nc12 ; O=C1CCC(N2C(=O)c3ccc(F)cc3C2=O)C(=O)N1 ; N#Cc1cccc2ncc(-c3cnn([C@H]4C[C@H](CCCNc5ccc6c(c5)C(=O)N(C5CCC(=O)NC5=O)C6=O)C4)c3)nc12]

GS-197 was synthesized following the similar procedure for preparing GS-168 (5 mg, yield: 11.3%). MS (ESI) m/z=589.4 [M+H]⁺.

### Example 156. 3-(1-(trans-3-(3-((2-(2,6-Dioxopiperidin-3-yl)-1,3-dioxoisoindolin-5-yl)amino)propyl)cyclobutyl)-1H-pyrazol-4-yl)quinoxaline-6-carbonitrile (GS-198)

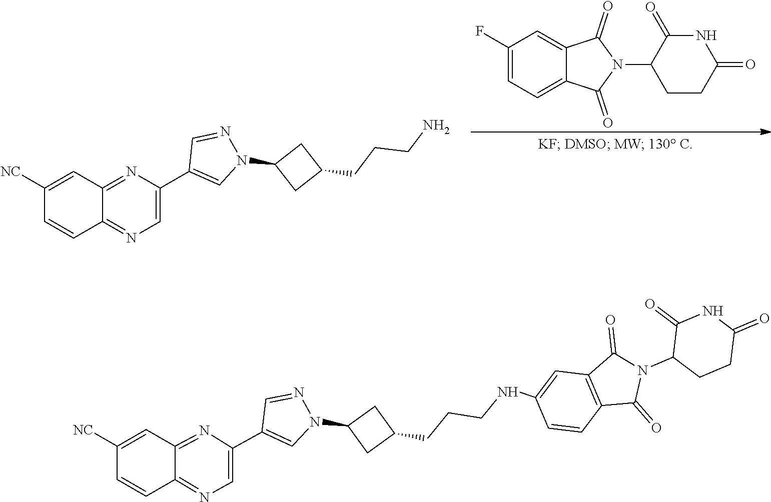 [SMILES: O=C1CCC(N2C(=O)c3ccc(F)cc3C2=O)C(=O)N1 ; N#Cc1ccc2ncc(-c3cnn([C@H]4C[C@H](CCCN)C4)c3)nc2c1 ; N#Cc1ccc2ncc(-c3cnn([C@H]4C[C@H](CCCNc5ccc6c(c5)C(=O)N(C5CCC(=O)NC5=O)C6=O)C4)c3)nc2c1]

GS-198 was synthesized following the similar procedure for preparing GS-168 (4 mg, yield: 15%). MS (ESI) m/z=589.5 [M+H]⁺.

### Example 157. 2-(2,6-Dioxopiperidin-3-yl)-5-((3-(trans-3-(4-(7-(piperidin-4-yl)quinoxalin-2-yl)-1H-pyrazol-1-yl)cyclobutyl)propyl)amino)isoindoline-1,3-dione (GS-199)

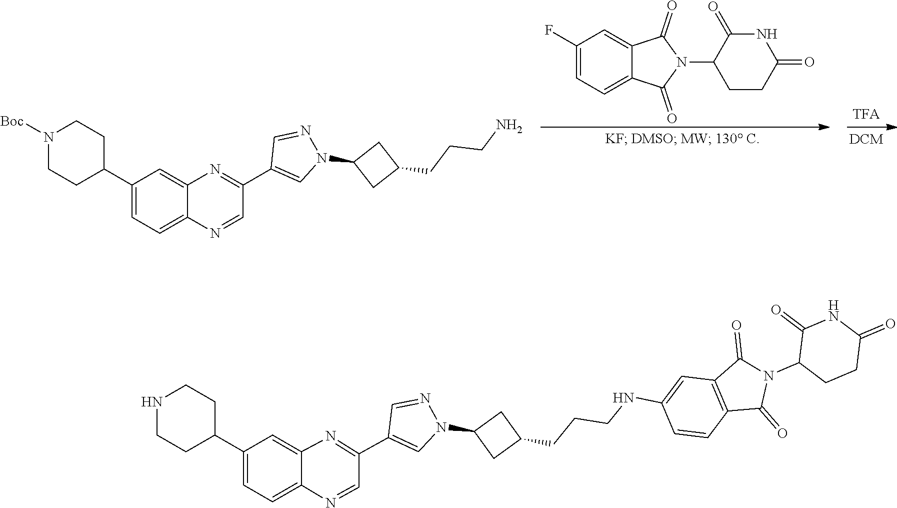 [SMILES: O=C1CCC(N2C(=O)c3ccc(F)cc3C2=O)C(=O)N1 ; CC(C)(C)OC(=O)N1CCC(c2ccc3ncc(-c4cnn([C@H]5C[C@H](CCCN)C5)c4)nc3c2)CC1 ; O=C1CCC(N2C(=O)c3ccc(NCCC[C@H]4C[C@H](n5cc(-c6cnc7ccc(C8CCNCC8)cc7n6)cn5)C4)cc3C2=O)C(=O)N1]

GS-199 was synthesized following the similar procedure for preparing GS-174 (23.5 mg, yield: 12.7%). MS (ESI) m/z=647.6 [M+H]⁺.

### Example 158. 2-(2,6-Dioxopiperidin-3-yl)-5-(((trans-3-(3-methyl-4-(quinoxalin-2-yl)-1H-pyrazol-1-yl)cyclobutyl)methyl)amino)isoindoline-1,3-dione (GS-200)

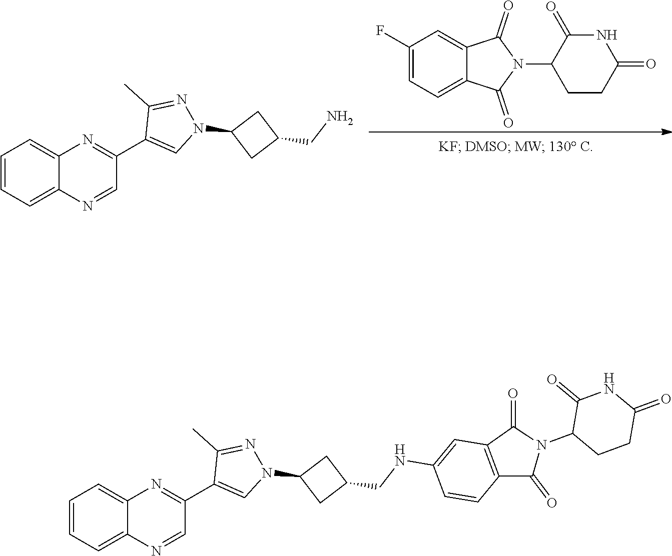

GS-200 was synthesized following the similar procedure for preparing GS-18 (0.8 mg, yield: 5.3%). MS (ESI) m/z=550.4 [M+H]⁺.

### Example 159. 2-(2,6-Dioxopiperidin-3-yl)-5-((3-(trans-3-(4-(7-fluoroquinoxalin-2-yl)-1H-pyrazol-1-yl)cyclobutyl)propyl)amino)isoindoline-1,3-dione (GS-201)

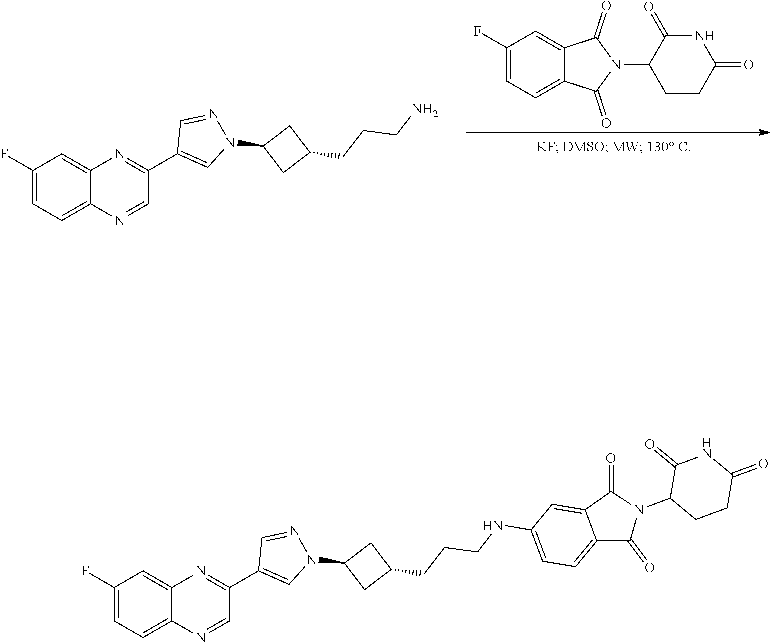

GS-201 was synthesized following the similar procedure or preparing GS-168 (30 mg, yield: 13.9%). MS (ESI) m/z=582.5 [M+H]⁺.

### Example 160. 5-((3-(trans-3-(4-(7-Cyclopropoxyquinoxalin-2-yl)-1H-pyrazol-1-yl)cyclobutyl)propyl)amino)-2-(2,6-dioxopiperidin-3-yl)isoindoline-1,3-dione (GS-202)

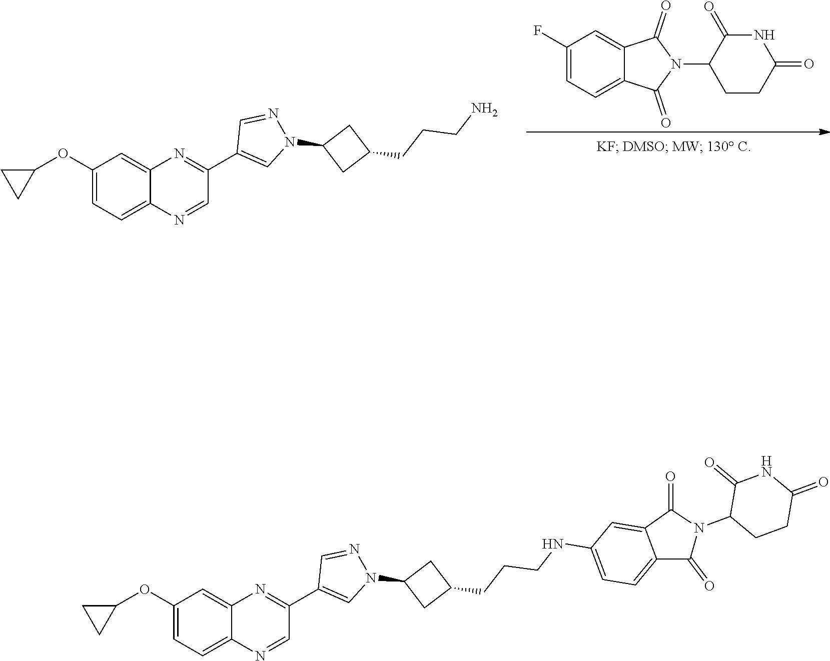

GS-202 was synthesized following the similar procedure for preparing GS-168 (10.2 mg, yield: 13.2%). MS (ESI) m/z=620.5 [M+H]⁺.

### Example 161. 5-((3-(trans-3-(4-(7-((1S,4S)-2,5-Diazabicyclo[2.2.1]heptan-2-yl)quinoxalin-2-yl)-1H-pyrazol-1-yl)cyclobutyl)propyl)amino)-2-(2,6-dioxopiperidin-3-yl)isoindoline-1,3-dione (GS-203)

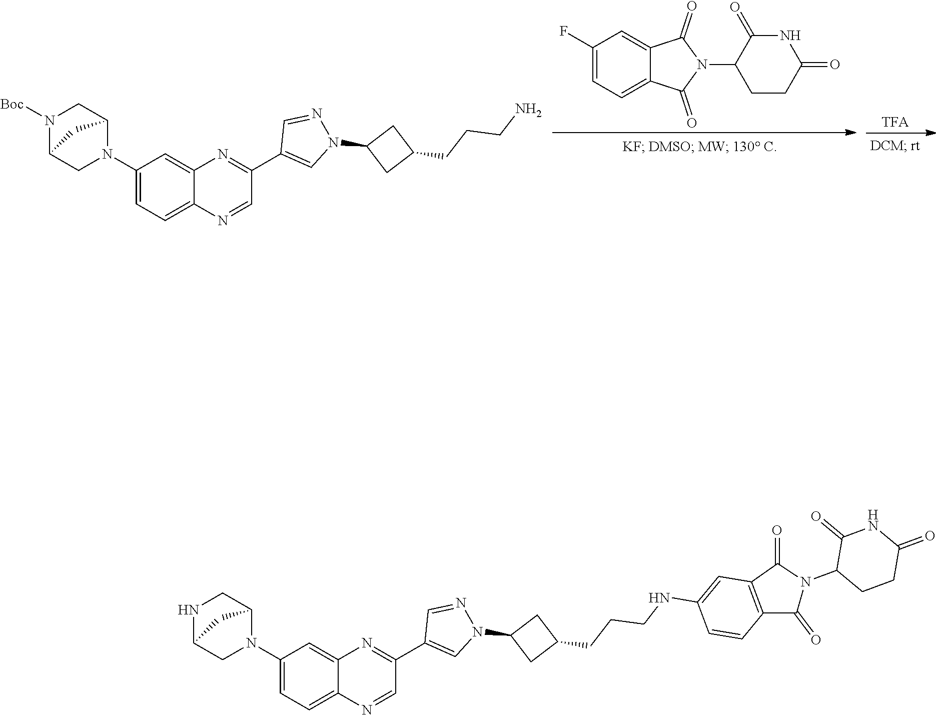

GS-203 was synthesized following the similar procedure for preparing GS-174 (1.2 mg, yield: 18.2%). MS (ESI) m/z=660.5 [M+H]⁺.

### Example 162. 2-(2,6-Dioxopiperidin-3-yl)-5-((3-(trans-3-(4-(7-(piperidin-4-yloxy)quinoxalin-2-yl)-1H-pyrazol-1-yl)cyclobutyl)propyl)amino)isoindoline-1,3-dione (GS-204)

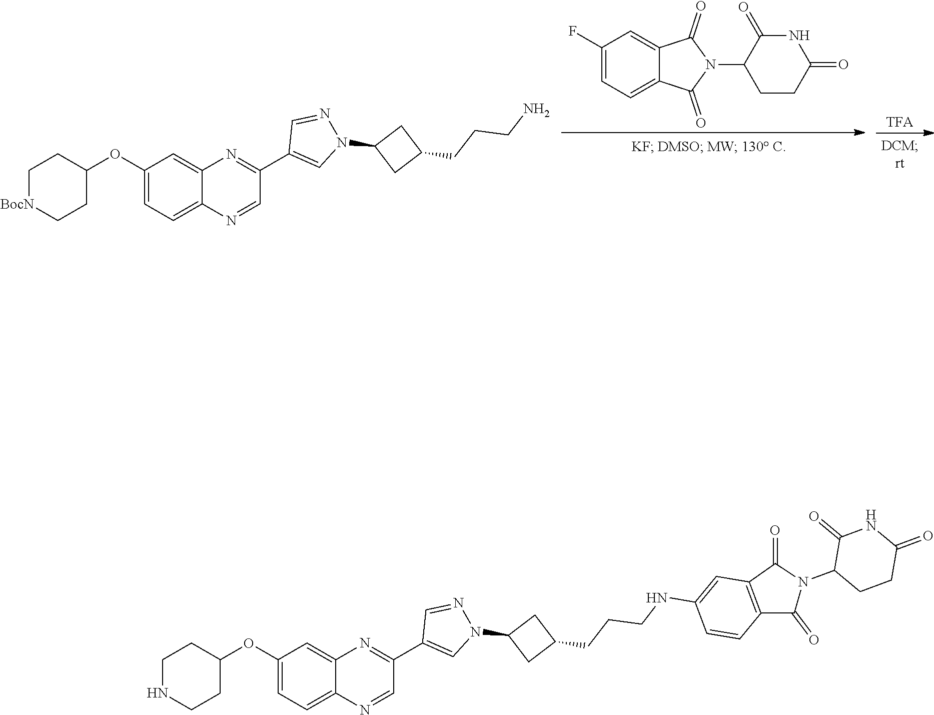

GS-204 was synthesized following the similar procedure or preparing GS-174 (36 mg, yield: 14%). MS (ESI) m/z=663.6 [M+H]⁺.

### Example 163. 2-(2,6-Dioxopiperidin-3-yl)-5-((2-(3-(4-(4-(quinoxalin-2-yl)-1H-pyrazol-1-yl)piperidine-1-carbonyl)cyclobutyl)ethyl)amino)isoindoline-1,3-dione (GS-205)

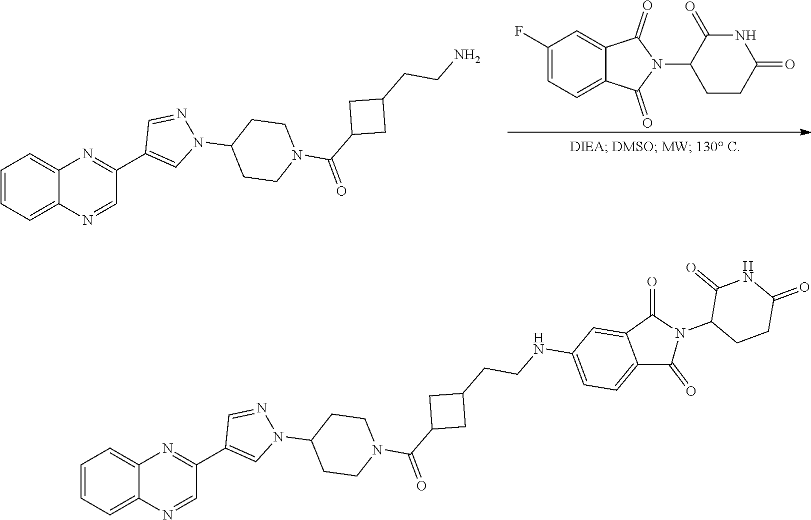

GS-205 was synthesized following the similar procedure for preparing GS-107 (43.5 mg, yield: 19.4%). MS (ESI) m/z=661.6 [M+H]⁺.

### Example 164. 5-((3-(trans-3-(4-(7-Chloroquinoxalin-2-yl)-1H-pyrazol-1-yl)cyclobutyl)propyl)amino)-2-(2,6-dioxopiperidin-3-yl)isoindoline-1,3-dione (GS-206)

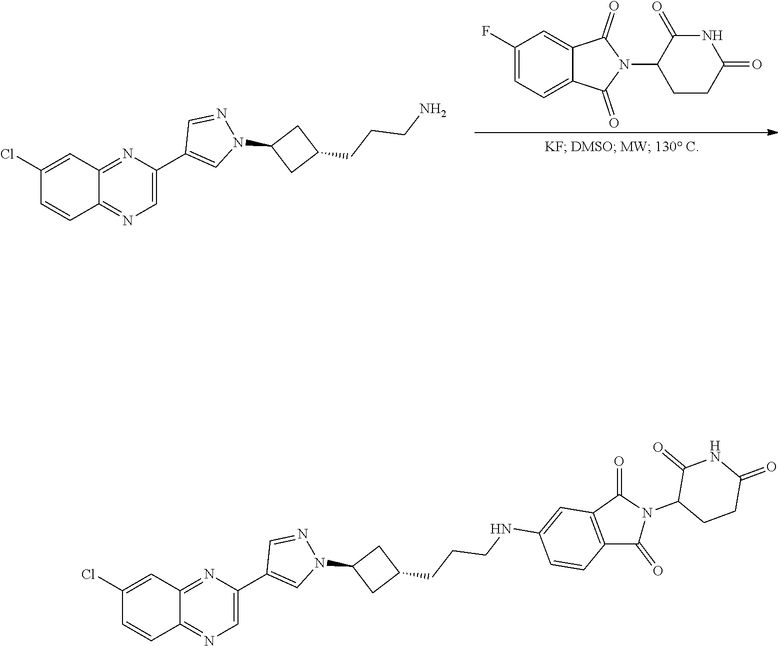

GS-206 was synthesized following the similar procedure for preparing GS-107 (16.4 mg, yield: 23.4%). MS (ESI) m/z=598.4 [M+H]⁺.

### Example 165. 2-(2,6-Dioxopiperidin-3-yl)-5-((3-(cis-3-(4-(7-morpholinoquinoxalin-2-yl)-1H-pyrazol-1-yl)cyclobutyl)propyl)amino)isoindoline-1,3-dione (GS-207)

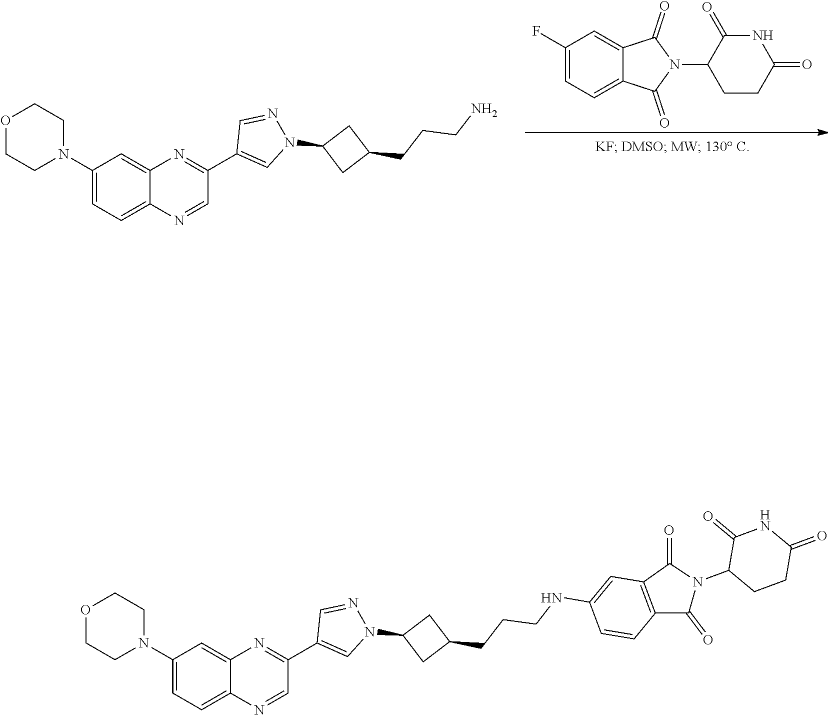

GS-207 was synthesized following the similar procedure for preparing GS-168 (21.3 mg, yield: 12.9%). MS (ESI) m/z=649.6 [M+H]⁺.

### Example 166. 2-(2,6-Dioxopiperidin-3-yl)-5-((3-(trans-3-(4-(7-(morpholinomethyl)quinoxalin-2-yl)-1H-pyrazol-1-yl)cyclobutyl)propyl)amino)isoindoline-1,3-dione (GS-208)

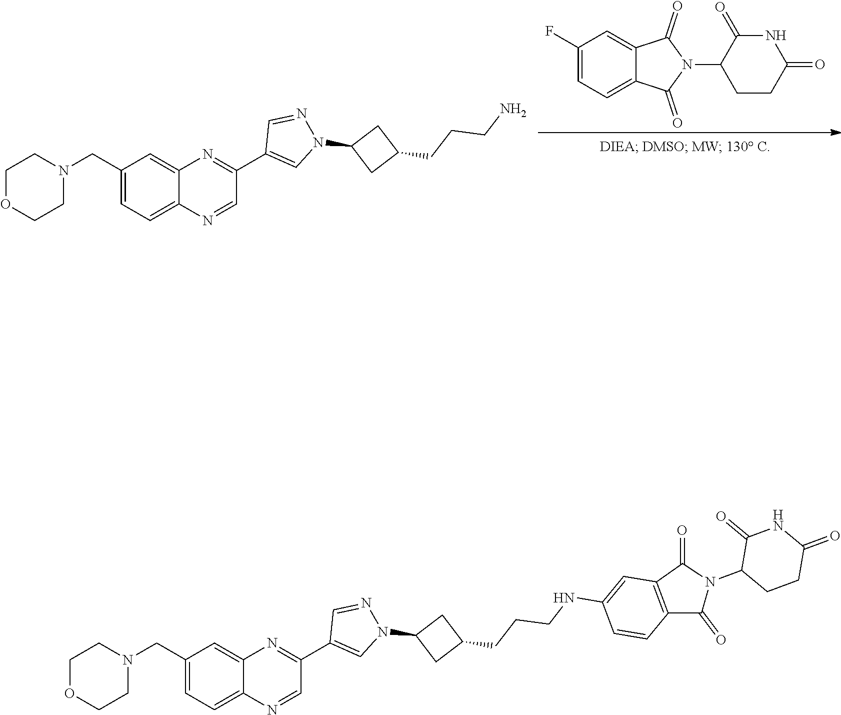

GS-208 was synthesized following the similar procedure for preparing GS-168 (15 mg, yield: 11.8%). MS (ESI) m/z=663.6 [M+H]⁺.

### Example 167. 2-(2,6-Dioxopiperidin-3-yl)-5-((3-(trans-3-(4-(7-(piperazin-1-ylmethyl)quinoxalin-2-yl)-1H-pyrazol-1-yl)cyclobutyl)propyl)amino)isoindoline-1,3-dione (GS-209)

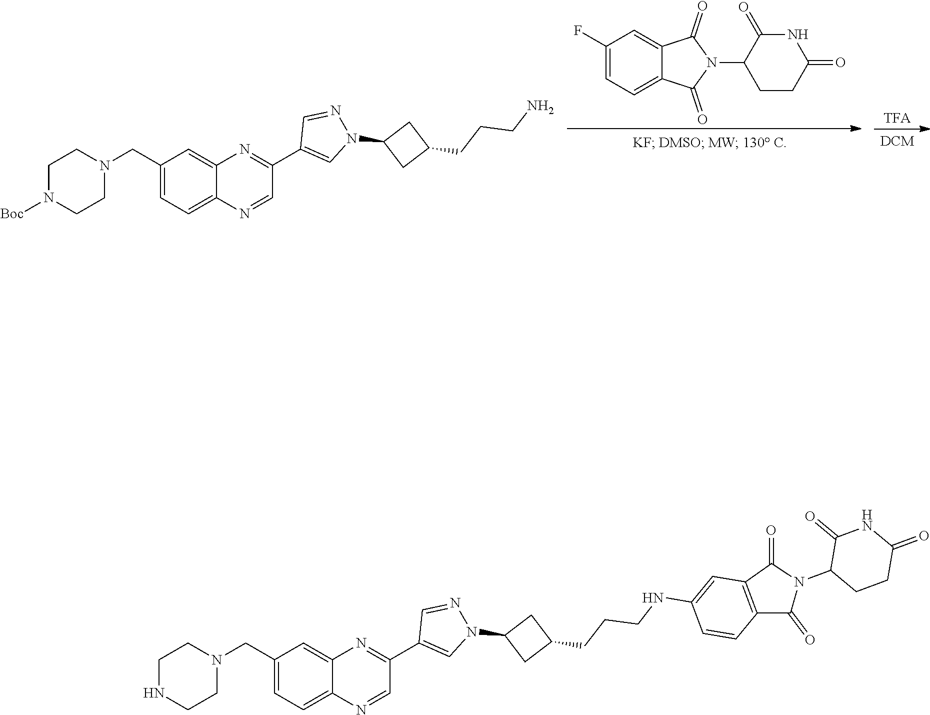

GS-209 was synthesized following the similar procedure for preparing GS-174 (25 mg, yield: 16.3%). MS (ESI) m/z=662.6 [M+H]⁺.

### Example 168. 2-(2,6-Dioxopiperidin-3-yl)-5-((3-(cis-3-(4-(7-(tetrahydro-2H-pyran-4-yl)quinoxalin-2-yl)-3-(trifluoromethyl)-1H-pyrazol-1-yl)cyclobutyl)propyl)amino)isoindoline-1,3-dione (GS-210)

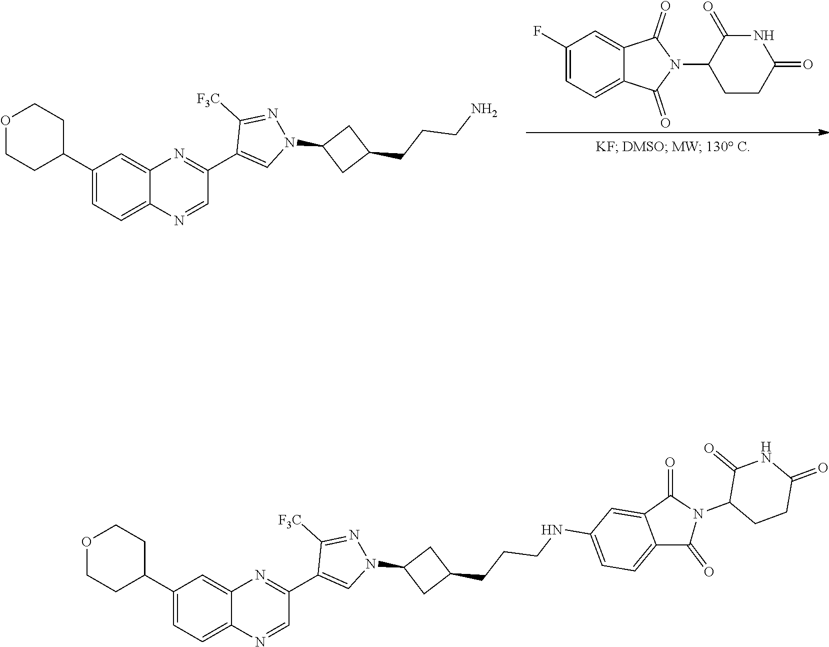

GS-210 was synthesized following the similar procedure for preparing GS-168 (2.2 mg, yield: 30.8%). MS (ESI) m/z=716.7 [M+H]⁺.

### Example 169. 5-((5-(4-(3-Cyclopropyl-4-(7-(piperazin-1-yl)quinoxalin-2-yl)-1H-pyrazol-1-yl)piperidin-1-yl)-5-oxopentyl)amino)-2-(2,6-dioxopiperidin-3-yl)isoindoline-1,3-dione (GS-211)

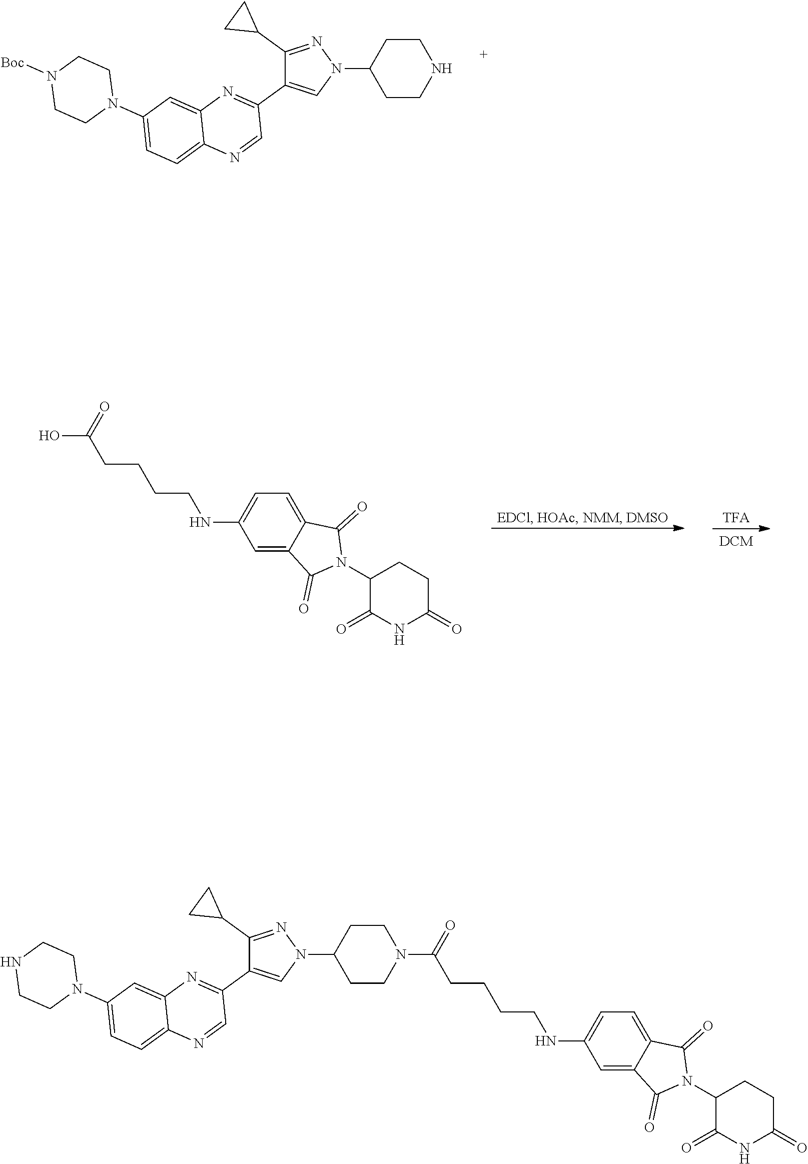 [SMILES: CC(C)(C)OC(=O)N1CCN(c2ccc3ncc(-c4cn(C5CCNCC5)nc4C4CC4)nc3c2)CC1 ; O=C(O)CCCCNc1ccc2c(c1)C(=O)N(C1CCC(=O)NC1=O)C2=O ; "EDCl, HOAc, NMM, DMSO" ; O=C1CCC(N2C(=O)c3ccc(NCCCCC(=O)N4CCC(n5cc(-c6cnc7ccc(N8CCNCC8)cc7n6)c(C6CC6)n5)CC4)cc3C2=O)C(=O)N1]

GS-211 was synthesized following the similar procedure for preparing GS-001 followed with Boc-deprotection (120 mg, yield: 37.4%). MS (ESI) m/z=759.8 [M+H]⁺.

### Example 170. 2-(2,6-Dioxopiperidin-3-yl)-5-((3-(trans-3-(4-(7-(trifluoromethyl)quinoxalin-2-yl)-1H-pyrazol-1-yl)cyclobutyl)propyl)amino)isoindoline-1,3-dione (GS-212)

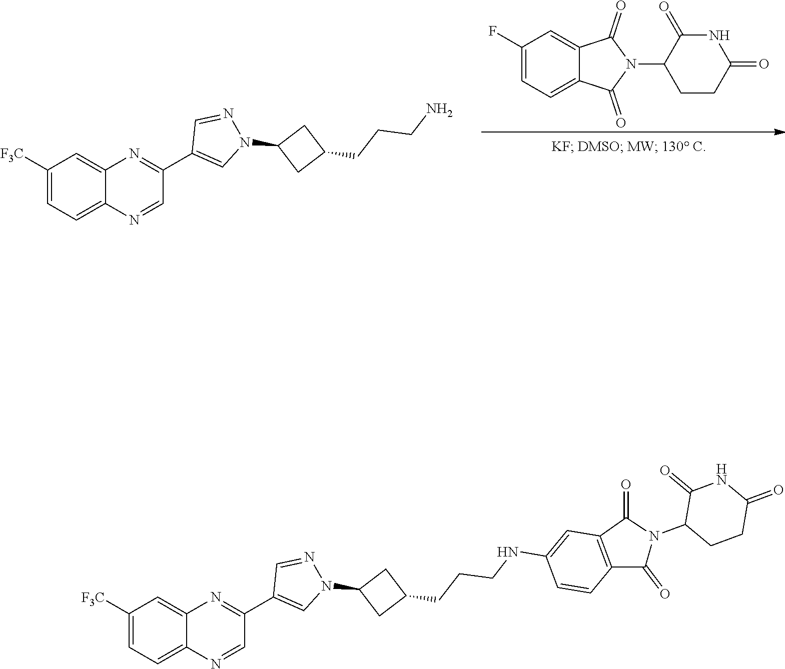

GS-212 was synthesized following the similar procedure for preparing GS-168 (15.2 mg, yield: 16.7%). MS (ESI) m/z=632.5 [M+H]⁺.

### Example 171. 5-((3-(cis-3-(3-Cyclopropyl-4-(7-(tetrahydro-2H-pyran-4-yl)quinoxalin-2-yl)-1H-pyrazol-1-yl)cyclobutyl)propyl)amino)-2-(2,6-dioxopiperidin-3-yl)isoindoline-1,3-dione (GS-213)

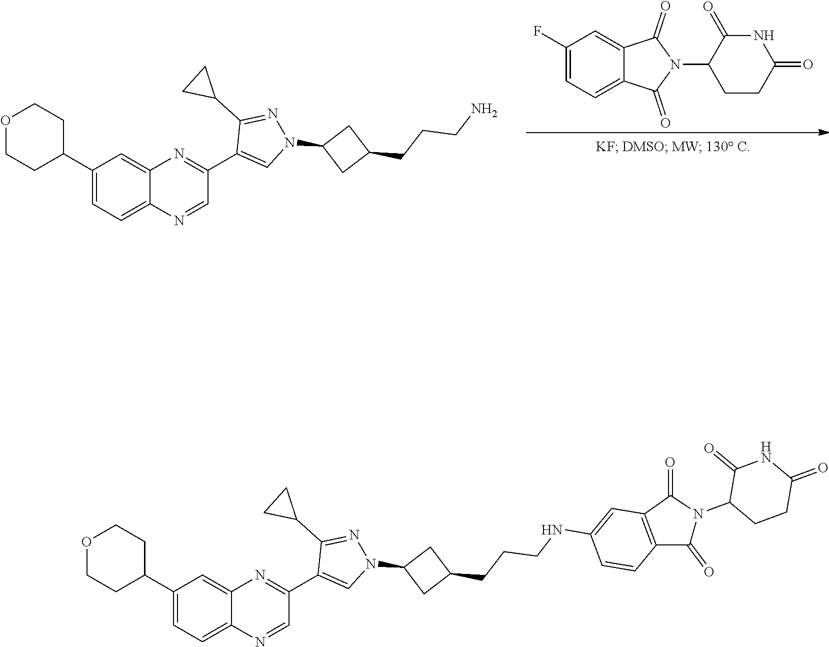

GS-213 was synthesized following the similar procedure for preparing GS-168 (5.6 mg, yield: 12.2%). MS (ESI) m/z=688.7 [M+H]⁺.

### Example 172. 2-(2,6-Dioxopiperidin-3-yl)-5-((3-(cis-3-(4-(7-(piperidin-4-ylamino)quinoxalin-2-yl)-1H-pyrazol-1-yl)cyclobutyl)propyl)amino)isoindoline-1,3-dione (GS-214)

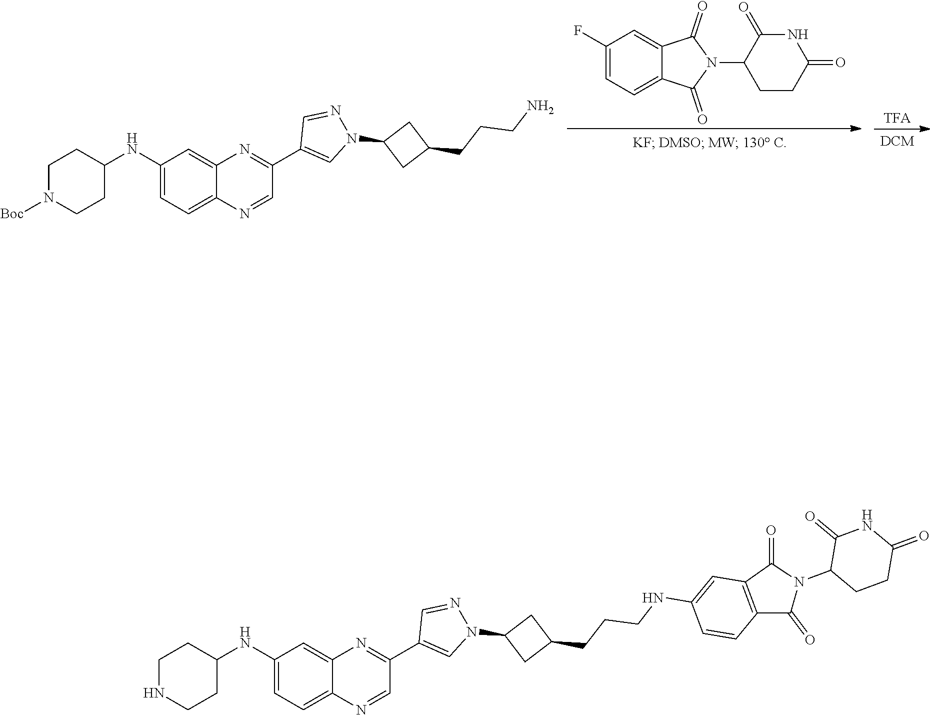

GS-214 was synthesized following the similar procedure or preparing GS-174 (4.2 mg, yield: 14%). MS (ESI) m/z=662.5 [M+H]⁺.

### Example 173. 5-((3-(trans-3-(4-(7-(3,8-Diazabicyclo[3.2.1]octan-3-yl)quinoxalin-2-yl)-1H-pyrazol-1-yl)cyclobutyl)propyl)amino)-2-(2,6-dioxopiperidin-3-yl)isoindoline-1,3-dione (GS-215)

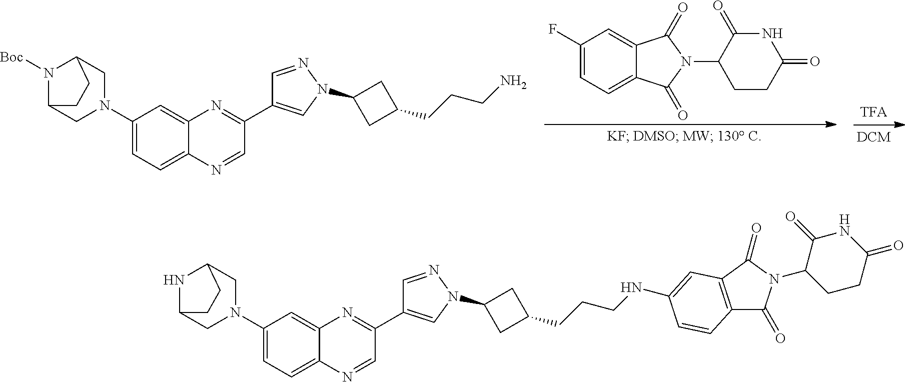 [SMILES: CC(C)(C)OC(=O)N1C2CCC1CN(c1ccc3ncc(-c4cnn([C@H]5C[C@H](CCCN)C5)c4)nc3c1)C2 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccc(N8CC9CCC(C8)N9)cc7n6)cn5)C4)cc3C2=O)C(=O)N1]

GS-215 was synthesized following the similar procedure for preparing GS-174 (3 mg, yield: 6.9%). MS (ESI) m/z=674.6 [M+H]⁺.

### Example 174. 2-(2,6-Dioxopiperidin-3-yl)-5-(((trans-3-(4-(6-morpholinopyridin-2-yl)-1H-pyrazol-1-yl)cyclobutyl)methyl)amino)isoindoline-1,3-dione (GS-216)

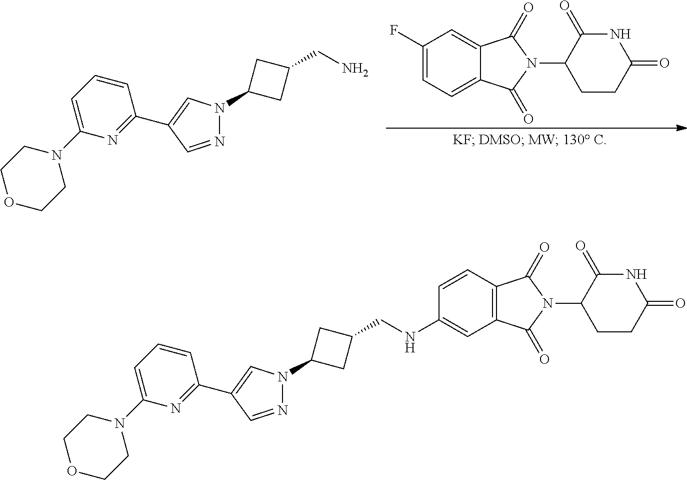 [SMILES: NC[C@H]1C[C@H](n2cc(-c3cccc(N4CCOCC4)n3)cn2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cccc(N7CCOCC7)n6)cn5)C4)cc3C2=O)C(=O)N1]

GS-216 was synthesized following the similar procedure or preparing GS-178 (12 mg, yield: 17.5%). MS (ESI) m/z=570.4 [M+H]⁺.

### Example 175. 2-(2,6-Dioxopiperidin-3-yl)-5-((3-(trans-3-(4-(6-morpholinopyridin-2-yl)-1H-pyrazol-1-yl)cyclobutyl)propyl)amino)isoindoline-1,3-dione (GS-217)

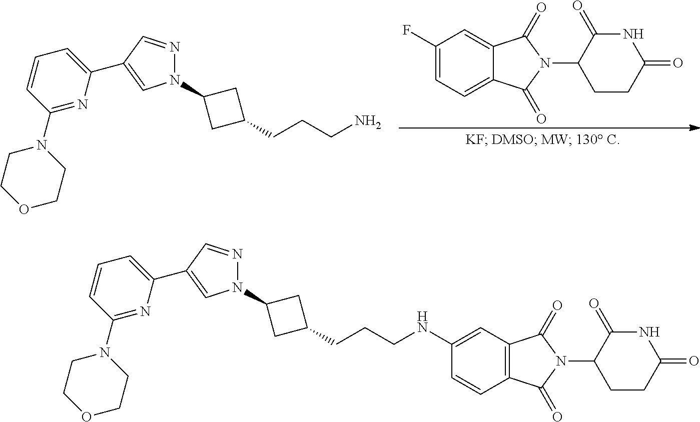 [SMILES: NCCC[C@H]1C[C@H](n2cc(-c3cccc(N4CCOCC4)n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cccc(N7CCOCC7)n6)cn5)C4)cc3C2=O)C(=O)N1]

GS-217 was synthesized following the similar procedure for preparing GS-168 (6.1 mg, yield: 22.3%). MS (ESI) m/z=598.5 [M+H]⁺.

### Example 176. 2-(2,6-Dioxopiperidin-3-yl)-5-(((trans-3-(4-(6-((tetrahydro-2H-pyran-4-yl)amino)pyridin-2-yl)-1H-pyrazol-1-yl)cyclobutyl)methyl)amino)isoindoline-1,3-dione (GS-218)

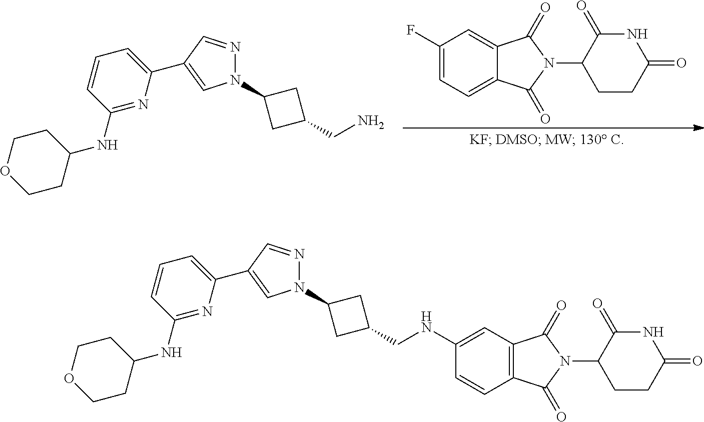 [SMILES: NC[C@H]1C[C@H](n2cc(-c3cccc(NC4CCOCC4)n3)cn2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cccc(NC7CCOCC7)n6)cn5)C4)cc3C2=O)C(=O)N1]

GS-218 was synthesized following the similar procedure for preparing GS-178 (7 mg, yield: 7.7%). MS (ESI) m/z=584.5 [M+H]⁺.

### Example 177. 5-((3-(trans-3-(4-(5-Chloroquinoxalin-2-yl)-1H-pyrazol-1-yl)cyclobutyl)propyl)amino)-2-(2,6-dioxopiperidin-3-yl)isoindoline-1,3-dione (GS-219)

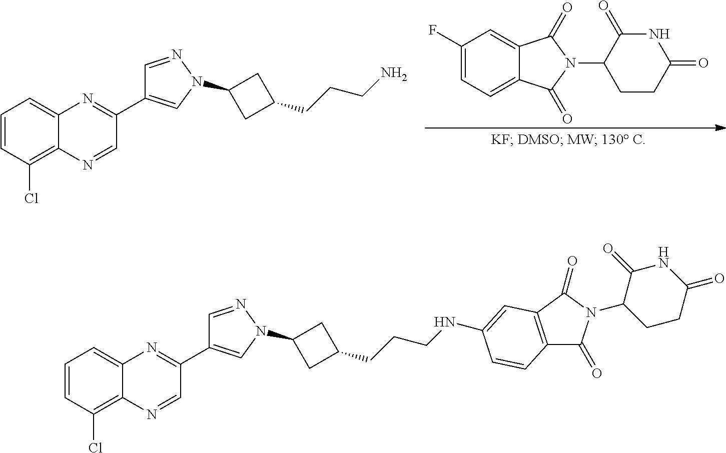 [SMILES: NCCC[C@H]1C[C@H](n2cc(-c3cnc4c(Cl)cccc4n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7c(Cl)cccc7n6)cn5)C4)cc3C2=O)C(=O)N1]

GS-219 was synthesized following the similar procedure for preparing GS-168 (15 mg, yield: 24%). MS (ESI) m/z=598.4 [M+H]⁺.

### Example 178. 5-((3-(trans-3-(4-(7-(4,7-Diazaspiro[2.5]octan-7-yl)quinoxalin-2-yl)-1H-pyrazol-1-yl)cyclobutyl)propyl)amino)-2-(2,6-dioxopiperidin-3-yl)isoindoline-1,3-dione (GS-220)

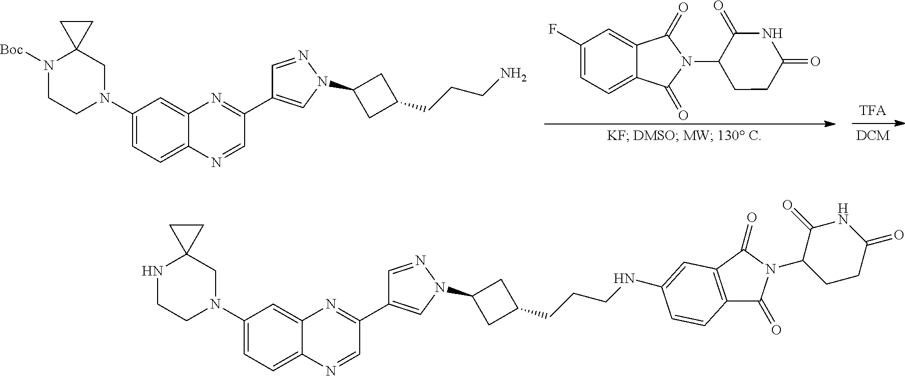 [SMILES: CC(C)(C)OC(=O)N1CCN(c2ccc3ncc(-c4cnn([C@H]5C[C@H](CCCN)C5)c4)nc3c2)CC12CC2 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccc(N8CCNC9(CC9)C8)cc7n6)cn5)C4)cc3C2=O)C(=O)N1]

GS-219 was synthesized following the similar procedure or preparing GS-174 (0.7 mg, yield: 0.33%). MS (ESI) m/z=674.6 [M+H]⁺.

### Example 179. 2-(2,6-Dioxopiperidin-3-yl)-5-((3-(cis-3-(3-methyl-4-(7-morpholinoquinoxalin-2-yl)-1H-pyrazol-1-yl)cyclobutyl)propyl)amino)isoindoline-1,3-dione (GS-221)

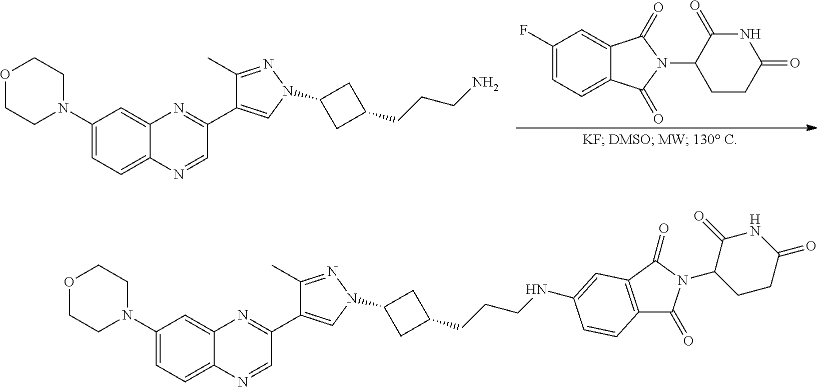 [SMILES: Cc1nn([C@H]2C[C@@H](CCCN)C2)cc1-c1cnc2ccc(N3CCOCC3)cc2n1 ; O=C1CCC(N2C(=O)c3ccc(F)cc3C2=O)C(=O)N1 ; Cc1nn([C@H]2C[C@@H](CCCNc3ccc4c(c3)C(=O)N(C3CCC(=O)NC3=O)C4=O)C2)cc1-c1cnc2ccc(N3CCOCC3)cc2n1]

GS-221 was synthesized following the similar procedure for preparing GS-168 (7.8 mg, yield: 47.8%). MS (ESI) m/z=663.6 [M+H]⁺.

### Example 180. 2-(2,6-Dioxopiperidin-3-yl)-5-((3-(trans-3-(4-(6-((tetrahydro-2H-pyran-4-yl)amino)pyridin-2-yl)-1H-pyrazol-1-yl)cyclobutyl)propyl)amino)isoindoline-3-dione (GS-222)

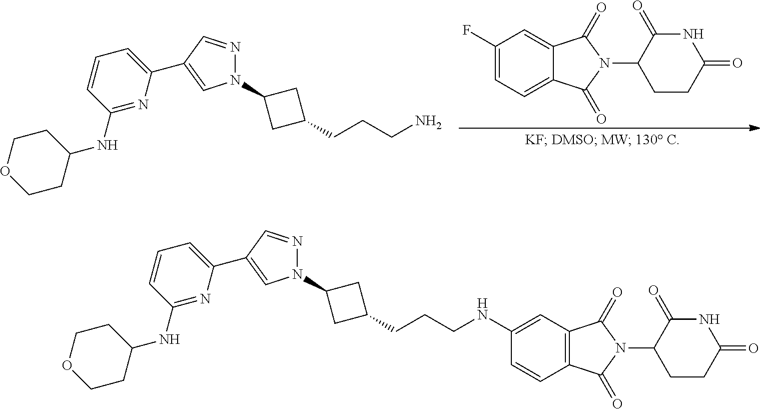 [SMILES: NCCC[C@H]1C[C@H](n2cc(-c3cccc(NC4CCOCC4)n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cccc(NC7CCOCC7)n6)cn5)C4)cc3C2=O)C(=O)N1]

GS-222 was synthesized following the similar procedure for preparing GS-168 (0.86 mg, yield: 1.7%). MS (ESI) m/z=612.6 [M+H]⁺.

### Example 181. 2-(2,6-Dioxopiperidin-3-yl)-5-((3-(cis-3-(3-methyl-4-(7-(tetrahydro-2H-pyran-4-yl)quinoxalin-2-yl)-1H-pyrazol-1-yl)cyclobutyl)propyl)amino)isoindoline-1,3-dione (GS-223)

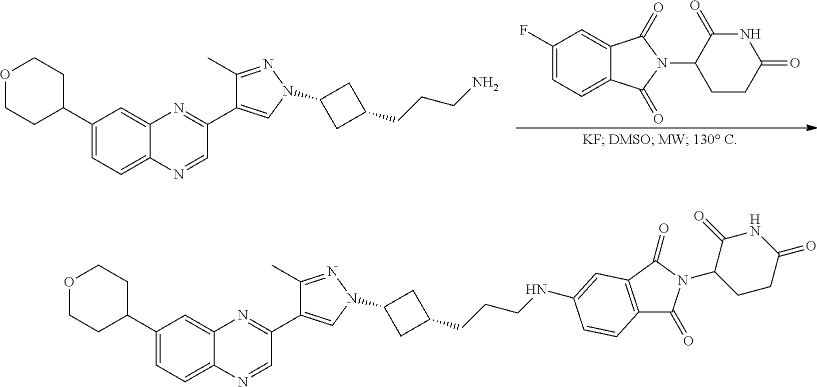 [SMILES: Cc1nn([C@H]2C[C@@H](CCCN)C2)cc1-c1cnc2ccc(C3CCOCC3)cc2n1 ; O=C1CCC(N2C(=O)c3ccc(F)cc3C2=O)C(=O)N1 ; Cc1nn([C@H]2C[C@@H](CCCNc3ccc4c(c3)C(=O)N(C3CCC(=O)NC3=O)C4=O)C2)cc1-c1cnc2ccc(C3CCOCC3)cc2n1]

GS-223 was synthesized following the similar procedure or preparing GS-168 (2.3 mg, yield: 5.6%). MS (ESI) m/z=662.6 [M+H]⁺.

### Example 182. 2-(2,6-Dioxopiperidin-3-yl)-5-((3-(trans-3-(4-(7-(1-methylpiperidin-4-yl)quinoxalin-2-yl)-1H-pyrazol-1-yl)cyclobutyl)propyl)amino)isoindoline-1,3-dione (GS-224)

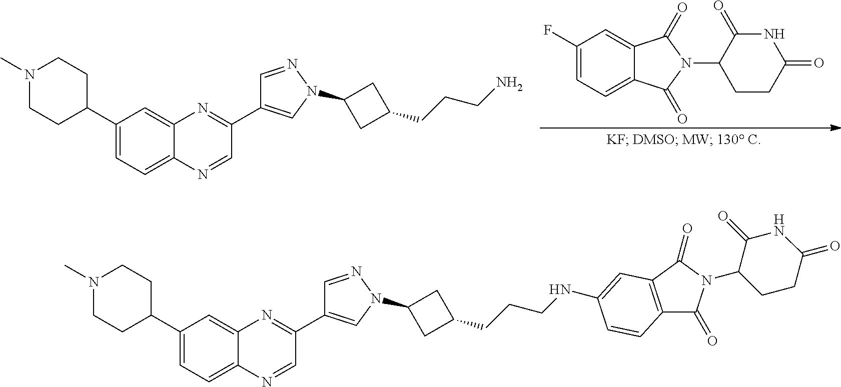 [SMILES: CN1CCC(c2ccc3ncc(-c4cnn([C@H]5C[C@H](CCCN)C5)c4)nc3c2)CC1 ; O=C1CCC(N2C(=O)c3ccc(F)cc3C2=O)C(=O)N1 ; CN1CCC(c2ccc3ncc(-c4cnn([C@H]5C[C@H](CCCNc6ccc7c(c6)C(=O)N(C6CCC(=O)NC6=O)C7=O)C5)c4)nc3c2)CC1]

GS-224 was synthesized following the similar procedure for preparing GS-168 (2.6 mg, yield: 13.3%). MS (ESI) m/z=661.6 [M+H]⁺.

### Example 183. 3-(1-Oxo-6-(4-(3-(4-(quinoxalin-2-yl)-1H-pyrazol-1-yl)cyclobutyl)butyl)isoindolin-2-yl)piperidine-2,6-dione (GS-225)

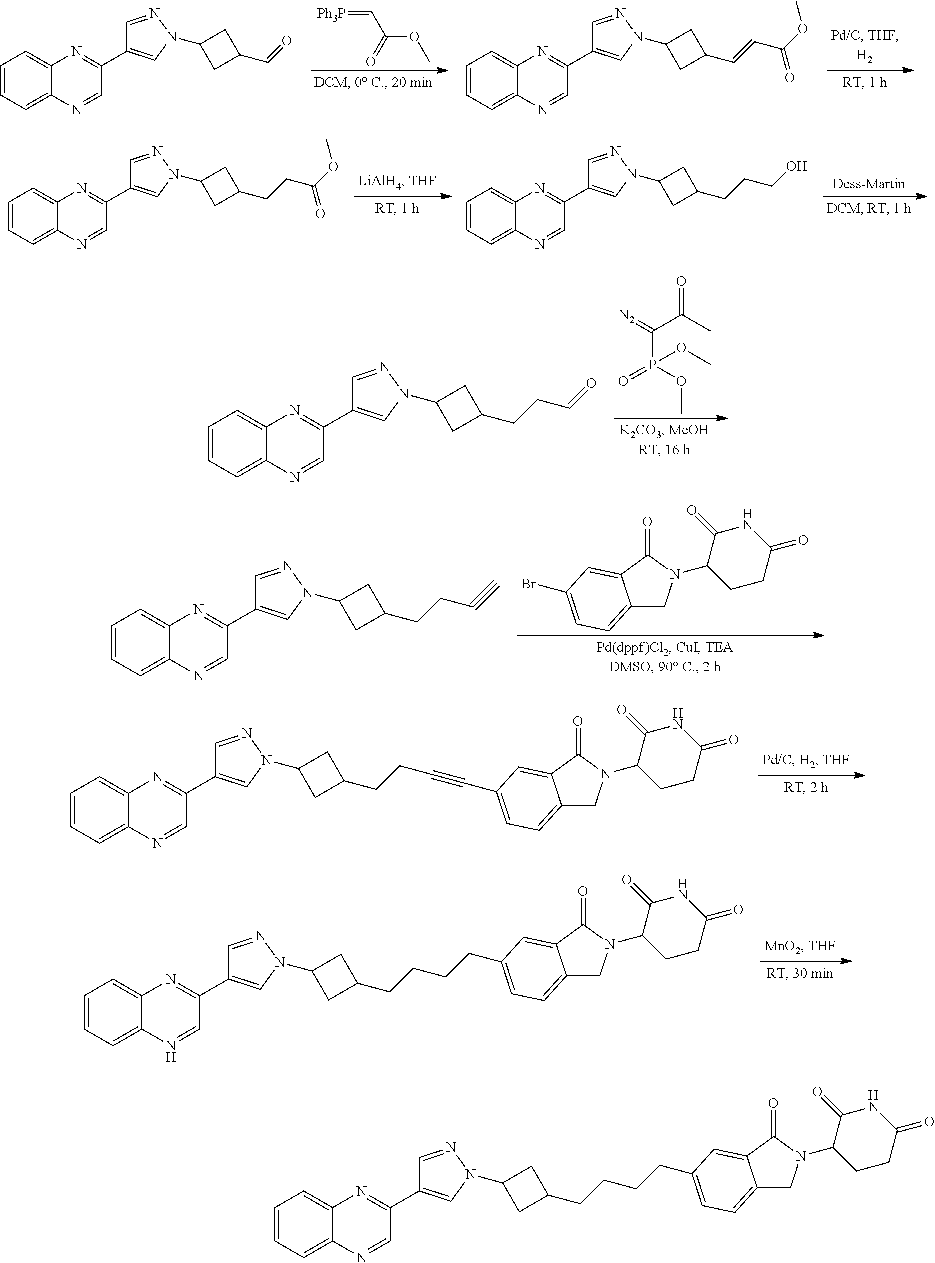

Step 1: Synthesis of methyl (E)-3-(3-(4-(quinoxalin-2-yl)-1H-pyrazol-1-yl)cyclobutyl)acrylate

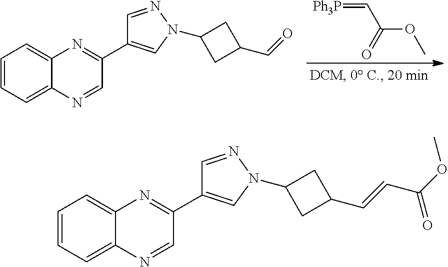

To a solution of 3-(4-(quinoxalin-2-yl)-1H-pyrazol-1-yl)cyclobutane-1-carbaldehyde (2.24 g, 8.07 mmol) in DCM (30.0 mL) was added methyl 2-(triphenyl-15-phosphanylidene)acetate (2.83 g, 8.47 mmol) in an ice-water bath. The reaction was stirred at 0° C. for 20 min, before it was poured into water and extracted with EtOAc (50 mL×3). The organic layers were combined, washed with brine, dried over Na₂SO₄, filtered, and concentrated. The resulting residue was purified by silica gel column chromatography (EtOAc/petroleum ether=3:5) to provide the title compound (2.5 g, 92.9% yield) as a white solid. MS (ESI) m/z=335.3 [M+H]⁺.

Step 2: Synthesis of methyl 3-(3-(4-(quinoxalin-2-yl)-1H-pyrazol-1-yl)cyclobutyl)propanoate

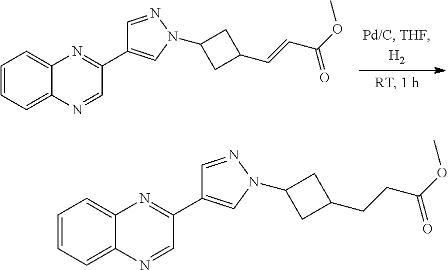 [SMILES: COC(=O)/C=C/C1CC(n2cc(-c3cnc4ccccc4n3)cn2)C1 ; COC(=O)CCC1CC(n2cc(-c3cnc4ccccc4n3)cn2)C1]

To a solution of methyl (E)-3-(3-(4-(quinoxalin-2-yl)-1H-pyrazol-1-yl)cyclobutyl)acrylate (2.5 g, 7.49 mmol) in THF (30.0 mL) was added Pd/C (700 mg, 15%). The reaction was stirred at rt for 1 h under H₂, before it was filtered. MnO₂ (4.0 g, 46.0 mmol) was added to the filtrate. And the resulting mixture was stirred at rt for 30 min, before it was filtered. The filtrate was concentrated. The resulting residue was purified by column chromatography (EtOAc/petroleum ether=3:5) to provide the title compound (846 mg, 33.7% yield) as a white solid. MS (ESI) m/z=337.3 [M+H]⁺.

Step 3: Synthesis of 3-(3-(4-(quinoxalin-2-yl)-1H-pyrazol-1-yl)cyclobutyl)propan-1-ol

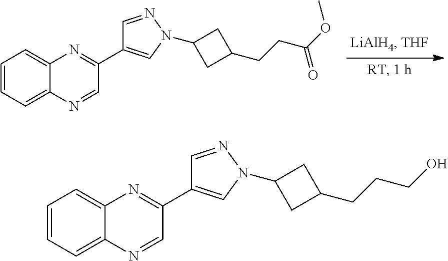 [SMILES: COC(=O)CCC1CC(n2cc(-c3cnc4ccccc4n3)cn2)C1 ; OCCCC1CC(n2cc(-c3cnc4ccccc4n3)cn2)C1]

To a solution of methyl 3-(3-(4-(quinoxalin-2-yl)-1H-pyrazol-1-yl)cyclobutyl)propanoate (0.843 mg, 2.51 mmol) in THF (10.0 mL) was added LiAlIH₄ (143 mg, 3.76 mmol) in an ice-water bath. The reaction was stirred at rt for 1 h, before it was quenched with H₂O (0.5 mL). The resulting mixture was stirred at rt for 10 min, before Na₂SO₄ was added, followed by MnO₂ (1.01 g, 12.55 mmol). After the mixture was stirred at rt for 30 min, the reaction was filtered. The filtrate was concentrated to provide the title compound (786 mg, 99% yield) as a colorless oil. MS (ESI) m/z=309.2 [M+H]⁺.

Step 4: Synthesis of 3-(3-(4-(quinoxalin-2-yl)-1H-pyrazol-1-yl)cyclobutyl)propanal

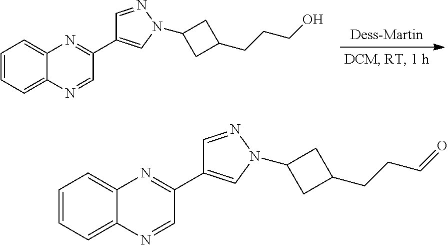 [SMILES: OCCCC1CC(n2cc(-c3cnc4ccccc4n3)cn2)C1 ; O=CCCC1CC(n2cc(-c3cnc4ccccc4n3)cn2)C1]

To a solution of 3-(3-(4-(quinoxalin-2-yl)-1H-pyrazol-1-yl)cyclobutyl)propan-1-ol (786 mg, 2.55 mmol) in DCM (8.0 mL) was added Dess-Martin Periodinane (1.3 g, 3.06 mmol). The reaction was stirred at rt for 1 h, before it was filtered. The filtrate was used directly in the next step. MS (ESI) m/z=307.2 [M+H]⁺.

Step 5: Synthesis of 2-(1-(3-(but-3-yn-1-yl)cyclobutyl)-1H-pyrazol-4-yl)quinoxaline

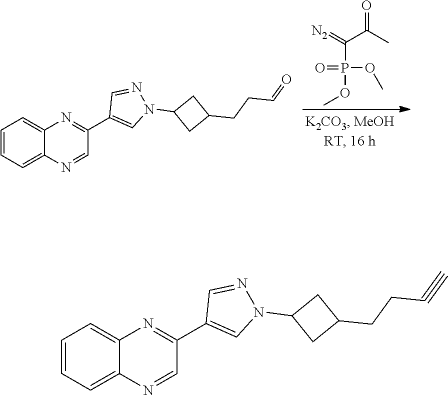

To a solution of 3-(3-(4-(quinoxalin-2-yl)-1H-pyrazol-1-yl)cyclobutyl)propanal (744 mg, 2.43 mmol) in MeOH (10.0 mL) were added dimethyl (1-diazo-2-oxopropyl)phosphonate (0.44 mL, 2.92 mmol) and K₂CO₃ (1.0 g, 7.29 mmol). After the reaction was stirred at rt for 16 h, it was poured into water and extracted with EtOAc (50 mL×3). The organic layers were combined, washed with brine, dried over Na₂SO₄, filtered, and concentrated. The resulting residue was purified by column chromatography (EtOAc/petroleum ether=62%) to provide the title compound (245 mg, 33.3% yield) as a white solid. MS (ESI) m/z=303.2 [M+H]⁺.

Step 6: Synthesis of 3-(1-oxo-6-(4-(3-(4-(quinoxalin-2-yl)-1H-pyrazol-1-yl)cyclobutyl)but-1-yn-1-yl)isoindolin-2-yl)piperidine-2,6-dione

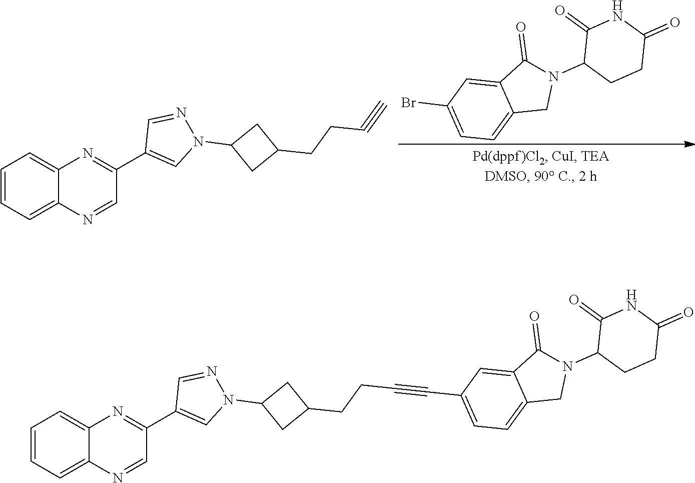

To a solution of 2-(1-(3-(but-3-yn-1-yl)cyclobutyl)-1H-pyrazol-4-yl)quinoxaline (50 mg, 0.166 mmol) and 3-(6-bromo-1-oxoisoindolin-2-yl)piperidine-2,6-dione (43 mg, 0.127 mmol) in DMSO (0.5 mL) were added Pd(dppf)Cl₂ (9.3 mg, 0.013 mmol), CuI (2.4 mg, 0.013 mmol) and TEA (0.177 mL, 1.27 mmol). The reaction mixture was stirred at 90° C. for 2 h under Ar. The mixture was purified by reverse phase chromatography to provide the title compound (46.9 mg, 51.9% yield) as a yellow solid. MS (ESI) m/z=545.5 [M+H]⁺.

Step 7: Synthesis of 3-(6-(4-(3-(4-(3,4-dihydroquinoxalin-2-yl)-1H-pyrazol-1-yl)cyclobutyl)butyl)-1-oxoisoindolin-2-yl)piperidine-2,6-dione

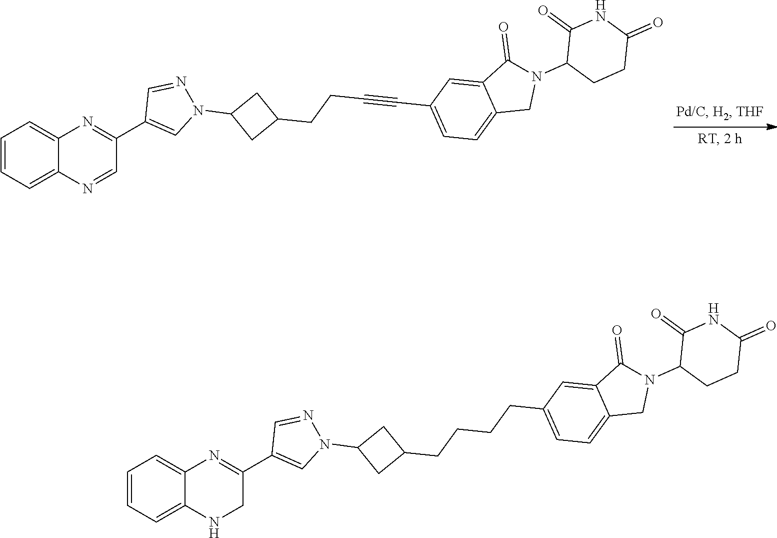 [SMILES: O=C1CCC(N2Cc3ccc(C#CCCC4CC(n5cc(-c6cnc7ccccc7n6)cn5)C4)cc3C2=O)C(=O)N1 ; O=C1CCC(N2Cc3ccc(CCCCC4CC(n5cc(C6=Nc7ccccc7NC6)cn5)C4)cc3C2=O)C(=O)N1]

To a solution of 3-(1-oxo-6-(4-(3-(4-(quinoxalin-2-yl)-1H-pyrazol-1-yl)cyclobutyl)but-1-yn-1-yl)isoindolin-2-yl)piperidine-2,6-dione (25 mg, 0.046 mmol) in THF (5.0 mL) was added Pd/C (10 mg, 10%). The reaction mixture was stirred at rt for 2 h under H₂, before it was filtered. The filtrate was concentrated to provide the crude product (23 mg, 90.8% yield) which was used directly in the next step. MS (ESI) m/z=551.5 [M+H]⁺.

Step 8: Synthesis of 3-(1-oxo-6-(4-(3-(4-(quinoxalin-2-yl)-1H-pyrazol-1-yl)cyclobutyl)butyl)isoindolin-2-yl)piperidine-2,6-dione

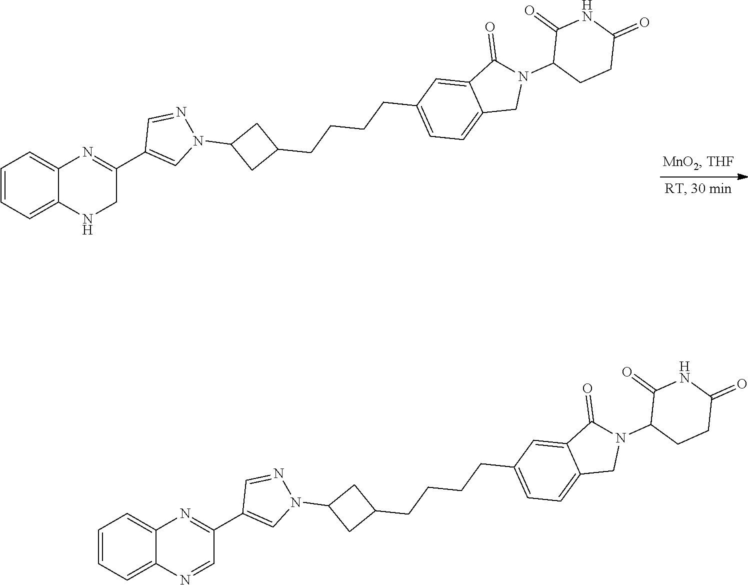 [SMILES: O=C1CCC(N2Cc3ccc(CCCCC4CC(n5cc(C6=Nc7ccccc7NC6)cn5)C4)cc3C2=O)C(=O)N1 ; O=C1CCC(N2Cc3ccc(CCCCC4CC(n5cc(-c6cnc7ccccc7n6)cn5)C4)cc3C2=O)C(=O)N1]

To a solution of 3-(6-(4-(3-(4-(3,4-dihydroquinoxalin-2-yl)-1H-pyrazol-1-yl)cyclobutyl)butyl)-1-oxoisoindolin-2-yl)piperidine-2,6-dione (23 mg, 0.042 mmol) in THF (2.0 mL) was added MnO₂ (20 mg, 0.23 mmol). The reaction was stirred at rt for 30 min, before it was filtered. The filtrate was concentrated and purified by reverse phase chromatography, followed by prep-TLC to provide the title compound (2.3 mg, 10% yield) as a yellow solid. MS (ESI) m/z=549.5 [M+H]⁻.

### Example 184. 3-(1-Oxo-5-(4-(3-(4-(quinoxalin-2-yl)-1H-pyrazol-1-yl)cyclobutyl)butyl)isoindolin-2-yl)piperidine-2,6-dione (GS-226)

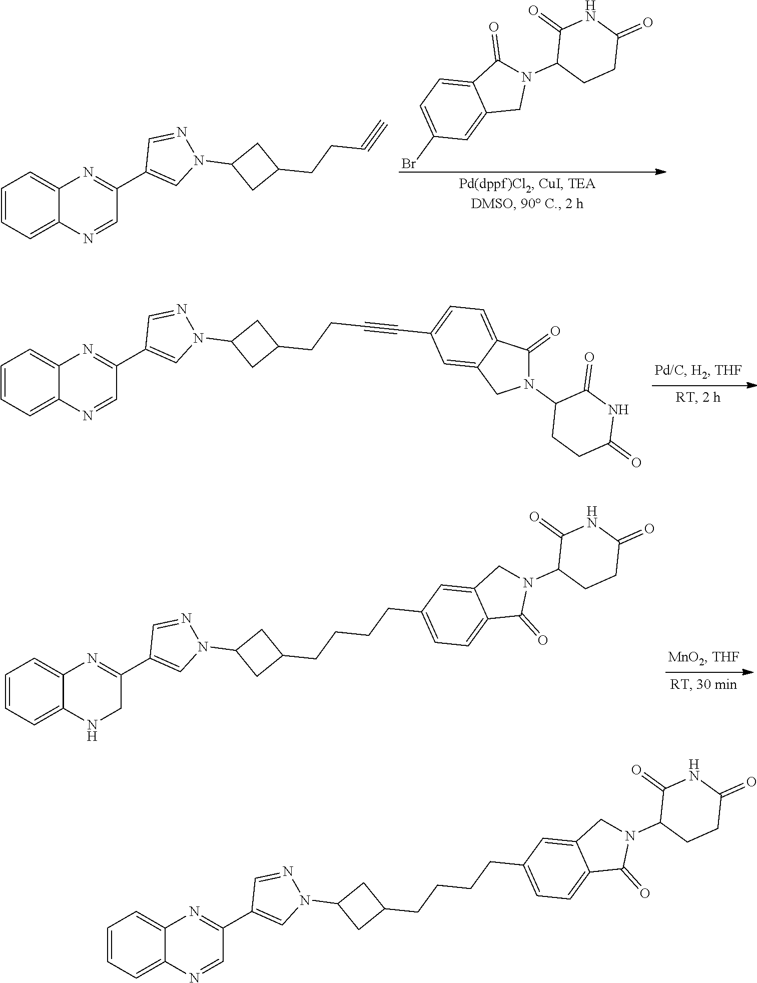

GS-226 was synthesized following the similar procedure for preparing GS-225 (3.4 mg, yield: 17%). MS (ESI) m/z=549.5 [M+H]⁺.

### Example 185. 5-((3-(cis-3-(3-Cyclopropyl-4-(7-morpholinoquinoxalin-2-yl)-1H-pyrazol-1-yl)cyclobutyl)propyl)amino)-2-(2,6-dioxopiperidin-3-yl)isoindoline-1,3-dione (GS-227)

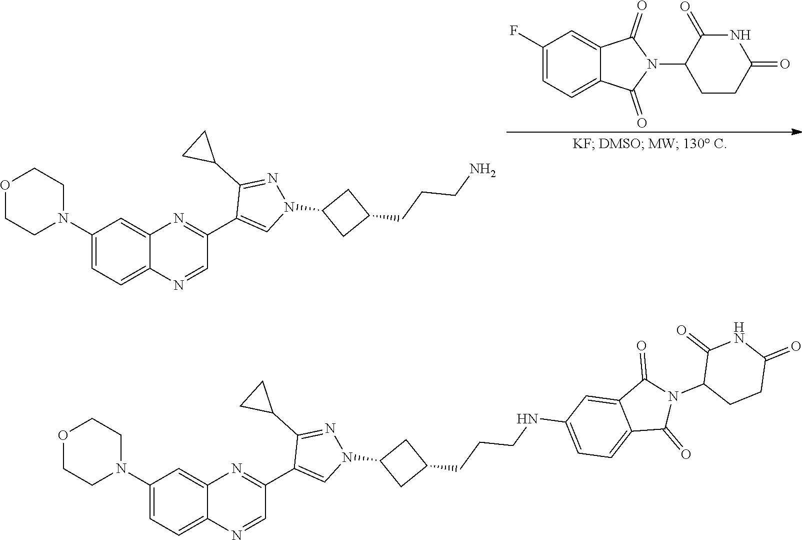 [SMILES: O=C1CCC(N2C(=O)c3ccc(F)cc3C2=O)C(=O)N1 ; NCCC[C@H]1C[C@@H](n2cc(-c3cnc4ccc(N5CCOCC5)cc4n3)c(C3CC3)n2)C1 ; O=C1CCC(N2C(=O)c3ccc(NCCC[C@H]4C[C@@H](n5cc(-c6cnc7ccc(N8CCOCC8)cc7n6)c(C6CC6)n5)C4)cc3C2=O)C(=O)N1]

GS-227 was synthesized following the similar procedure for preparing GS-168 (2.3 mg, yield: 6%). MS (ESI) m/z=689.7 [M+H]⁺.

### Example 186. 5-((3-(trans-3-(3-Cyclopropyl-4-(7-(piperazin-1-ylmethyl)quinoxalin-2-yl)-1H-pyrazol-1-yl)cyclobutyl)propyl)amino)-2-(2,6-dioxopiperidin-3-yl)isoindoline-1,3-dione (GS-228)

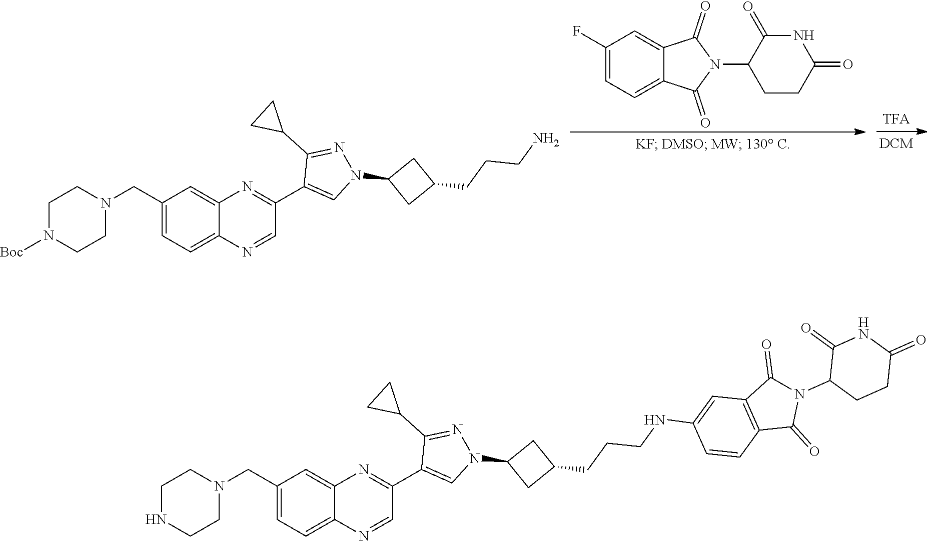 [SMILES: CC(C)(C)OC(=O)N1CCN(Cc2ccc3ncc(-c4cn([C@H]5C[C@H](CCCN)C5)nc4C4CC4)nc3c2)CC1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccc(CN8CCNCC8)cc7n6)c(C6CC6)n5)C4)cc3C2=O)C(=O)N1]

GS-228 was synthesized following the similar procedure for preparing GS-174 (4.7 mg, yield: 8%). MS (ESI) m/z=702.7 [M+H]⁺.

### Example 187. 5-((3-(trans-3-(3-Cyclopropyl-4-(7-(piperidin-4-yloxy)quinoxalin-2-yl)-1H-pyrazol-1-yl)cyclobutyl)propyl)amino)-2-(2,6-dioxopiperidin-3-yl)isoindoline-1,3-dione (GS-229)

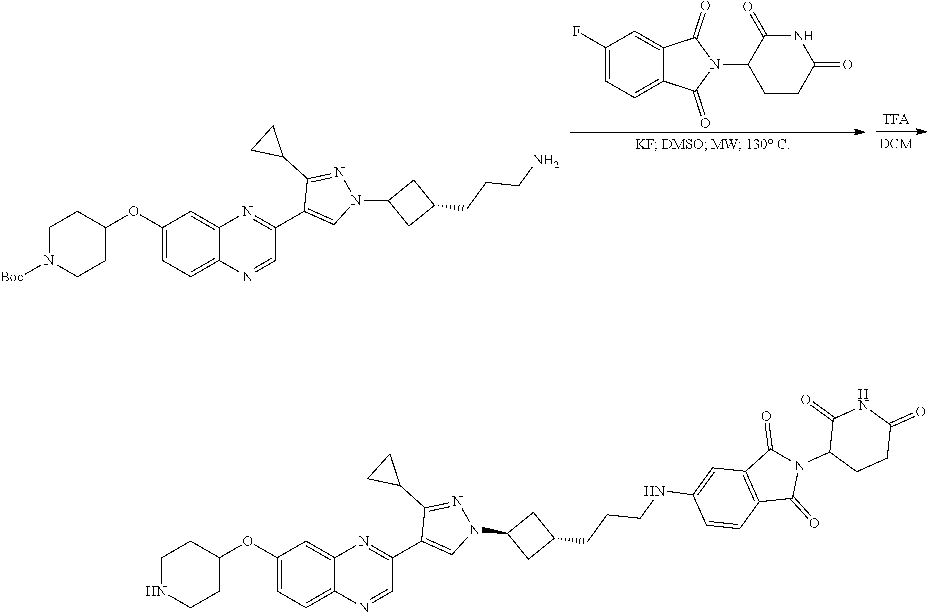

GS-229 was synthesized following the similar procedure for preparing GS-174 (1.1 mg, yield: 9.2%). MS (ESI) m/z=703.6 [M+H]⁺.

### Example 188. 5-((3-(trans-3-(3-Cyclopropyl-4-(7-(morpholinomethyl)quinoxalin-2-yl)-1H-pyrazol-1-yl)cyclobutyl)propyl)amino)-2-(2,6-dioxopiperidin-3-yl)isoindoline-1,3-dione GS230)

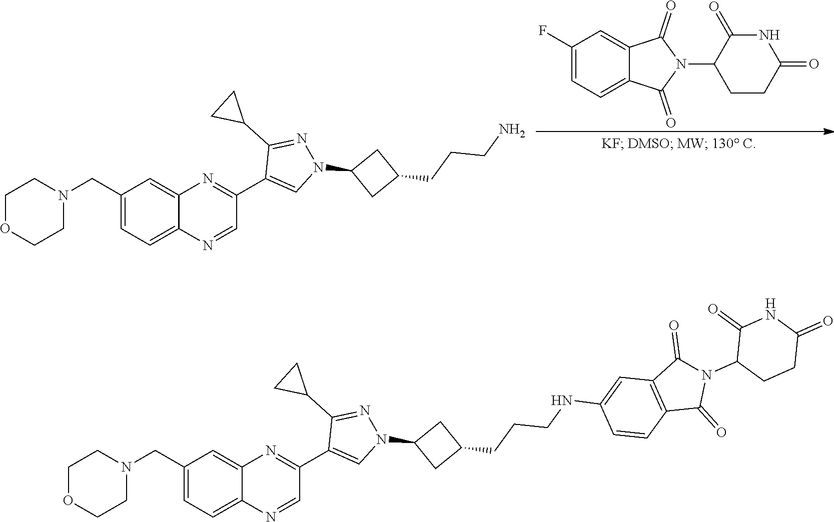 [SMILES: NCCC[C@H]1C[C@H](n2cc(-c3cnc4ccc(CN5CCOCC5)cc4n3)c(C3CC3)n2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccc(CN8CCOCC8)cc7n6)c(C6CC6)n5)C4)cc3C2=O)C(=O)N1]

GS-230 was synthesized following the similar procedure for preparing GS-168 (8.6 mg, yield: 21.9%). MS (ESI) m/z=703.6 [M+H]⁺.

### Example 189. 5-((3-(cis-3-(3-Cyclopropyl-4-(7-(piperidin-4-yl)quinoxalin-2-yl)-1H-pyrazol-1-yl)cyclobutyl)propyl)amino)-2-(2,6-dixopiperidin-3-yl)isoindoline-1,3-dione (GS-231)

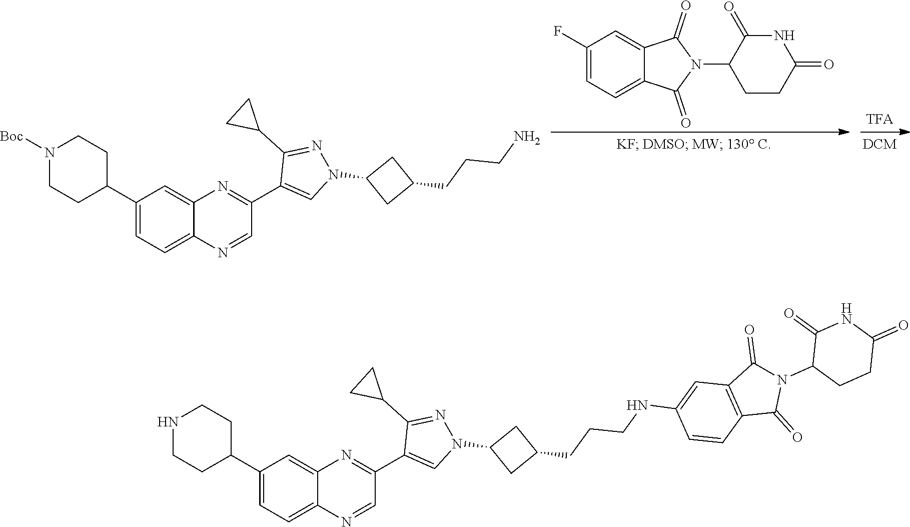 [SMILES: CC(C)(C)OC(=O)N1CCC(c2ccc3ncc(-c4cn([C@H]5C[C@@H](CCCN)C5)nc4C4CC4)nc3c2)CC1 ; O=C1CCC(N2C(=O)c3ccc(F)cc3C2=O)C(=O)N1 ; O=C1CCC(N2C(=O)c3ccc(NCCC[C@H]4C[C@@H](n5cc(-c6cnc7ccc(C8CCNCC8)cc7n6)c(C6CC6)n5)C4)cc3C2=O)C(=O)N1]

GS-230 was synthesized following the similar procedure for preparing GS-174 (1.3 mg, yield: 15%). MS (ESI) m/z=687.7 [M+H]⁺.

### Example 190. 2-(2,6-Dioxopiperidin-3-yl)-5-((3-(trans-3-(4-phenyl-1H-pyrazol-1-yl)cyclobutyl)propyl)amino)isoindoline-1,3-dione (GS-232)

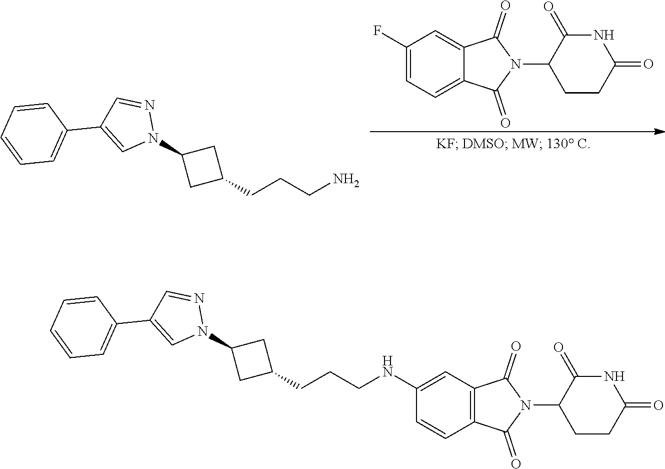 [SMILES: NCCC[C@H]1C[C@H](n2cc(-c3ccccc3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6ccccc6)cn5)C4)cc3C2=O)C(=O)N1]

GS-232 was synthesized following the similar procedure for preparing GS-168 (1.6 mg, yield: 5.3%). MS (ESI) m/z=512.5 [M+H]⁺.

### Example 191. 2-(1-(trans-3-(3-((2-(2,6-Dioxopiperidin-3-yl)-1,3-dioxoisoindolin-5-yl)amino)propyl)cyclobutyl)-1H-pyrazol-4-yl)quinoxaline-5-carbonitrile (GS-233)

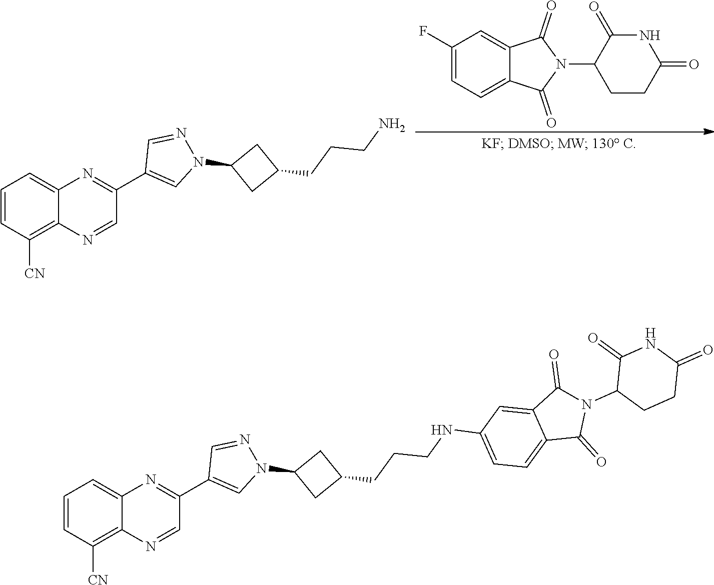 [SMILES: O=C1CCC(N2C(=O)c3ccc(F)cc3C2=O)C(=O)N1 ; N#Cc1cccc2nc(-c3cnn([C@H]4C[C@H](CCCN)C4)c3)cnc12 ; N#Cc1cccc2nc(-c3cnn([C@H]4C[C@H](CCCNc5ccc6c(c5)C(=O)N(C5CCC(=O)NC5=O)C6=O)C4)c3)cnc12]

GS-233 was synthesized following the similar procedure or preparing GS-168 (1.0 mg, yield: 5.6%). MS (ESI) m/z=589.4 [M+H]⁺.

### Example 192. 2-(2,6-Dioxopiperidin-3-yl)-5-((3-(trans-3-(4-(5-fluoroquinoxalin-2-yl)-1H-pyrazol-1-yl)cyclobutyl)propyl)amino)isoindoline-1,3-dione (GS-234)

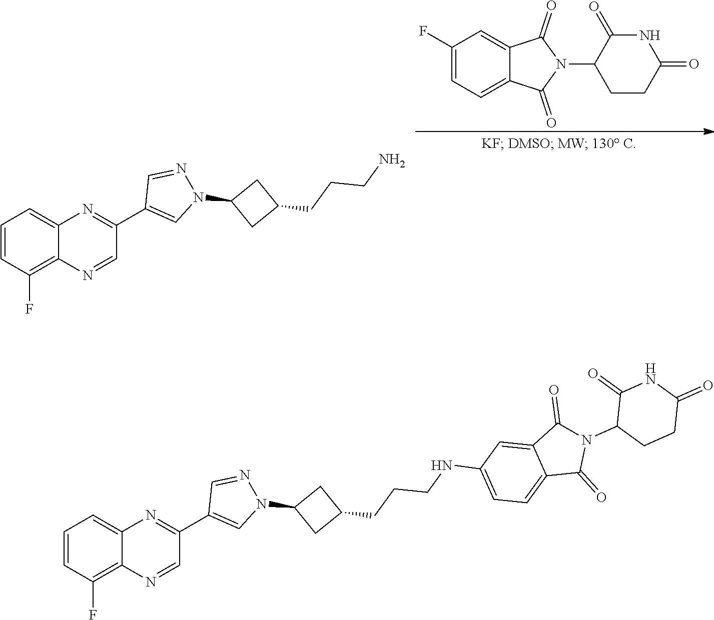 [SMILES: O=C1CCC(N2C(=O)c3ccc(F)cc3C2=O)C(=O)N1 ; NCCC[C@H]1C[C@H](n2cc(-c3cnc4c(F)cccc4n3)cn2)C1 ; O=C1CCC(N2C(=O)c3ccc(NCCC[C@H]4C[C@H](n5cc(-c6cnc7c(F)cccc7n6)cn5)C4)cc3C2=O)C(=O)N1]

GS-234 was synthesized following the similar procedure for preparing GS-168 (17 mg, yield: 11%). MS (ESI) m/z=582.4 [M+H]⁺.

### Example 193. 2-(2,6-Dioxopiperidin-3-yl)-5-((3-(trans-3-(4-(pyrazin-2-yl)-1H-pyrazol-1-yl)cyclobutyl)propyl)amino)isoindoline-1,3-dione (GS-235)

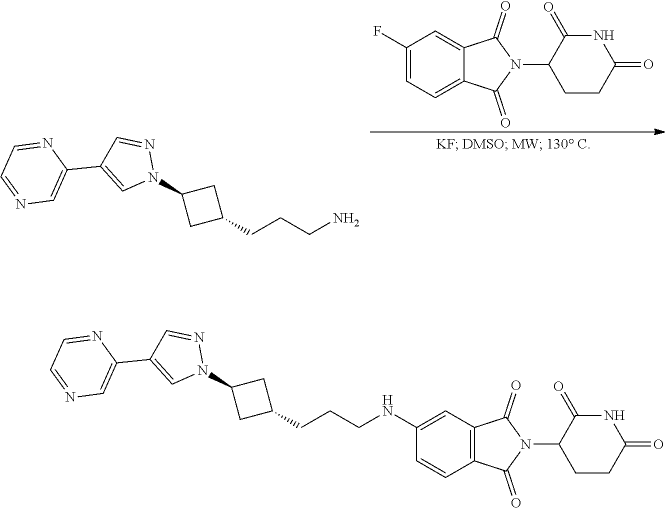 [SMILES: O=C1CCC(N2C(=O)c3ccc(F)cc3C2=O)C(=O)N1 ; NCCC[C@H]1C[C@H](n2cc(-c3cnccn3)cn2)C1 ; O=C1CCC(N2C(=O)c3ccc(NCCC[C@H]4C[C@H](n5cc(-c6cnccn6)cn5)C4)cc3C2=O)C(=O)N1]

GS-235 was synthesized following the similar procedure or preparing GS-168 (3.2 mg, yield: 10%). MS (ESI) m/z=514.4 [M+H]⁺.

### Example 194. 2-(2,6-Dioxopiperidin-3-yl)-5-((3-(trans-3-(4-(6-(tetrahydro-2H-pyran-4-yl)pyridin-2-yl)-1H-pyrazol-1-yl)cyclobutyl)propyl)amino)isoindoline-1,3-dione (GS-236)

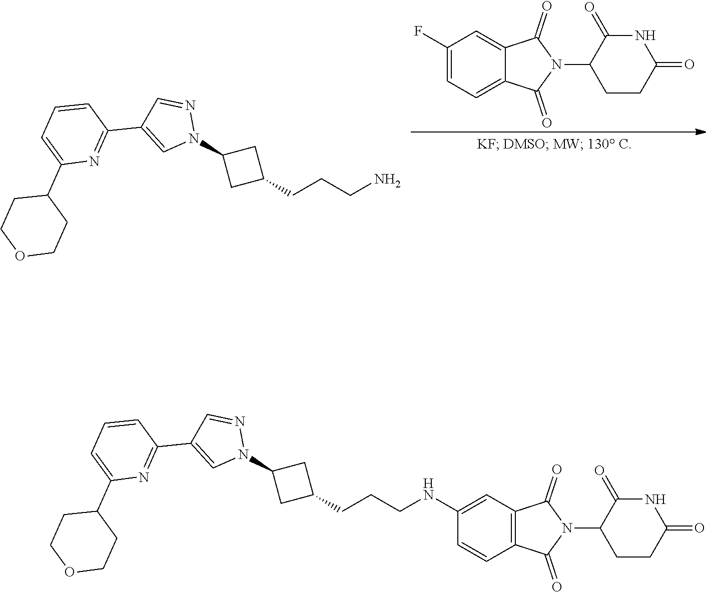 [SMILES: NCCC[C@H]1C[C@H](n2cc(-c3cccc(C4CCOCC4)n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cccc(C7CCOCC7)n6)cn5)C4)cc3C2=O)C(=O)N1]

GS-236 was synthesized following the similar procedure for preparing GS-168 (15 mg, yield: 17%). MS (ESI) m/z=597.6 [M+H]⁺.

### Example 195. 2-(2,6-Dioxopiperidin-3-yl)-5-((3-(trans-3-(4-(6-(1-methylpiperidin-4-yl)pyridin-2-yl)-1H-pyrazol-1-yl)cyclobutyl)propyl)amino)isoindoline-1,3-dione (GS-237)

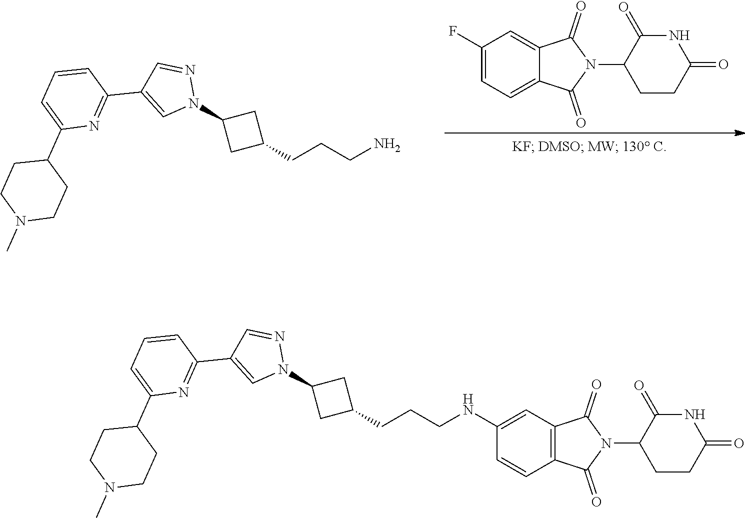 [SMILES: CN1CCC(c2cccc(-c3cnn([C@H]4C[C@H](CCCN)C4)c3)n2)CC1 ; O=C1CCC(N2C(=O)c3ccc(F)cc3C2=O)C(=O)N1 ; CN1CCC(c2cccc(-c3cnn([C@H]4C[C@H](CCCNc5ccc6c(c5)C(=O)N(C5CCC(=O)NC5=O)C6=O)C4)c3)n2)CC1]

GS-237 was synthesized following the similar procedure for preparing GS-168 (3 mg, yield: 22%). MS (ESI) m/z=610.5 [M+H]⁺.

### Example 196. 2-(2,6-Dioxopiperidin-3-yl)-5-((3-(trans-3-(4-(6-(piperidin-4-yl)pyridin-2-yl)-1H-pyrazol-1-yl)cyclobutyl)propyl)amino)isoindoline-1,3-dione (GS-238)

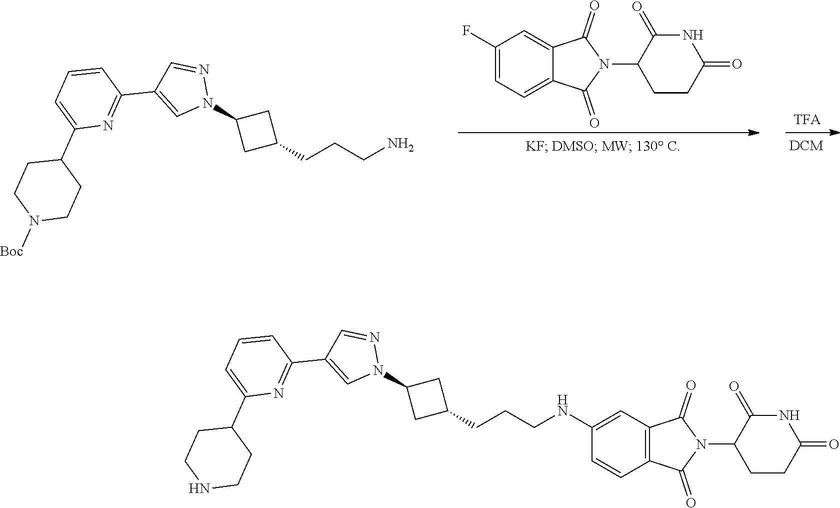 [SMILES: CC(C)(C)OC(=O)N1CCC(c2cccc(-c3cnn([C@H]4C[C@H](CCCN)C4)c3)n2)CC1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cccc(C7CCNCC7)n6)cn5)C4)cc3C2=O)C(=O)N1]

GS-238 was synthesized following the similar procedure for preparing GS-174 (3.5 mg, yield: 5.7%). MS (ESI) m/z=596.5 [M+H]⁺.

### Example 197. 2-(2,6-Dioxopiperidin-3-yl)-5-((3-(trans-3-(4-(2-morpholinopyrimidin-4-yl)-1H-pyrazol-1-yl)cyclobutyl)propyl)amino)isoindoline-1,3-dione (GS-239)

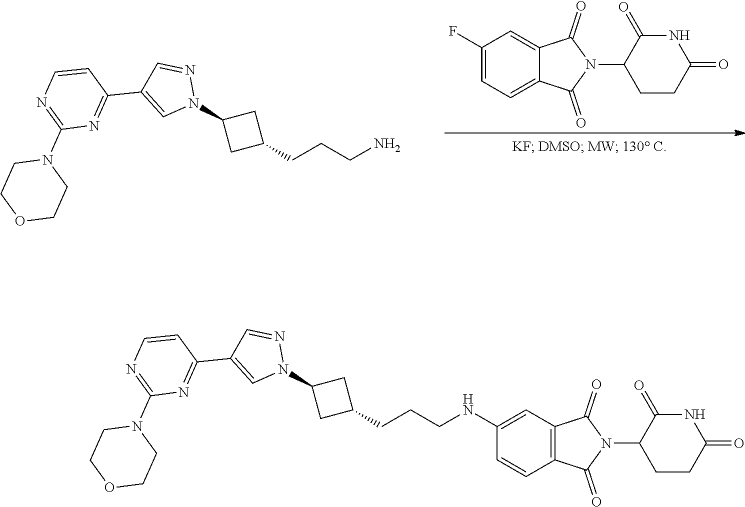 [SMILES: NCCC[C@H]1C[C@H](n2cc(-c3ccnc(N4CCOCC4)n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6ccnc(N7CCOCC7)n6)cn5)C4)cc3C2=O)C(=O)N1]

GS-239 was synthesized following the similar procedure for preparing GS-168 (115 mg, yield: 43.8%). MS (ESI) m/z=599.7 [M+H]⁺.

### Example 198. 2-(2,6-Dioxopiperidin-3-yl)-5-((3-(trans-3-(4-(4-morpholinopyrimidin-2-yl)-1H-pyrazol-1-yl)cyclobutyl)propyl)amino)isoindoline-1,3-dione (GS-240)

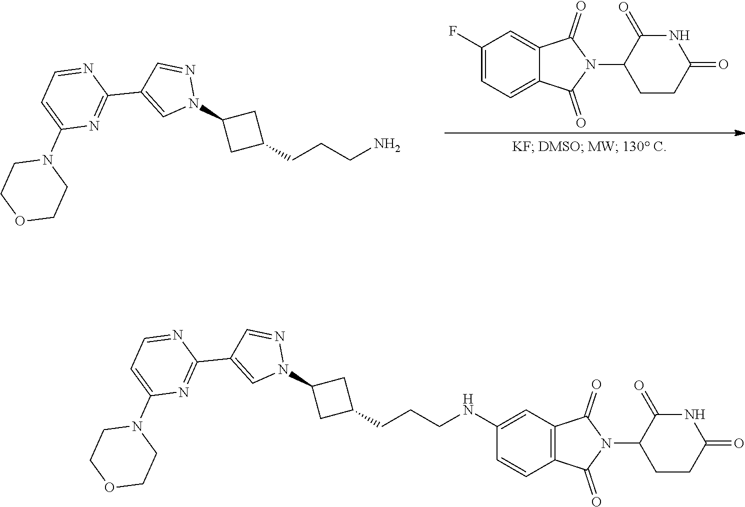 [SMILES: NCCC[C@H]1C[C@H](n2cc(-c3nccc(N4CCOCC4)n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6nccc(N7CCOCC7)n6)cn5)C4)cc3C2=O)C(=O)N1]

GS-240 was synthesized following the similar procedure for preparing GS-168 (230 mg, yield: 65.8%). MS (ESI) m/z=599.6 [M+H]⁺.

### Example 199. 2-(2,6-Dioxopiperidin-3-yl)-5-((3-(trans-3-(4-(3-morpholinophenyl)-1H-pyrazol-1-yl)cyclobutyl)propyl)amino)isoindoline-1,3-dione (GS-241)

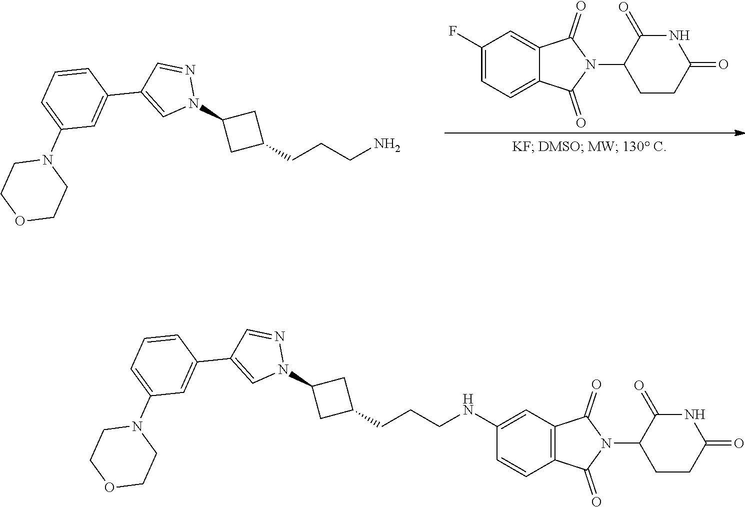 [SMILES: NCCC[C@H]1C[C@H](n2cc(-c3cccc(N4CCOCC4)c3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cccc(N7CCOCC7)c6)cn5)C4)cc3C2=O)C(=O)N1]

GS-241 was synthesized following the similar procedure for preparing GS-168 (3.1 mg, yield: 4.9%). MS (ESI) m/z=597.7 [M+H]⁺.

### Example 200. 2-(2,6-Dioxopiperidin-3-yl)-5-((3-(trans-3-(4-(pyrimidin-2-yl)-1H-pyrazol-1-yl)cyclobutyl)propyl)amino)isoindoline-1,3-dione (GS-242)

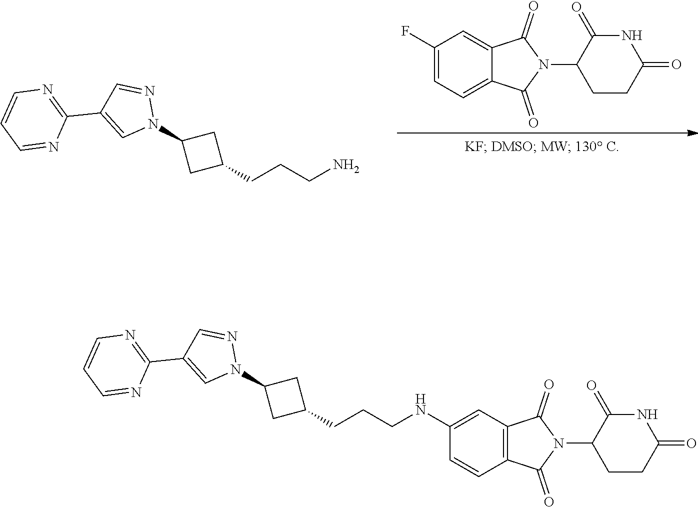 [SMILES: NCCC[C@H]1C[C@H](n2cc(-c3ncccn3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6ncccn6)cn5)C4)cc3C2=O)C(=O)N1]

GS-242 was synthesized following the similar procedure for preparing GS-168 (1.1 mg, yield: 16.7%). MS (ESI) m/z=514.4 [M+H]⁺.

### Example 201. 5-((3-(trans-3-(3-(Difluoromethyl)-4-(quinoxalin-2-yl)-1H-pyrazol-1-yl)cyclobutyl)propyl)amino)-2-(2,6-dioxopiperidin-3-yl)isoindoline-1,3-dione (GS-243)

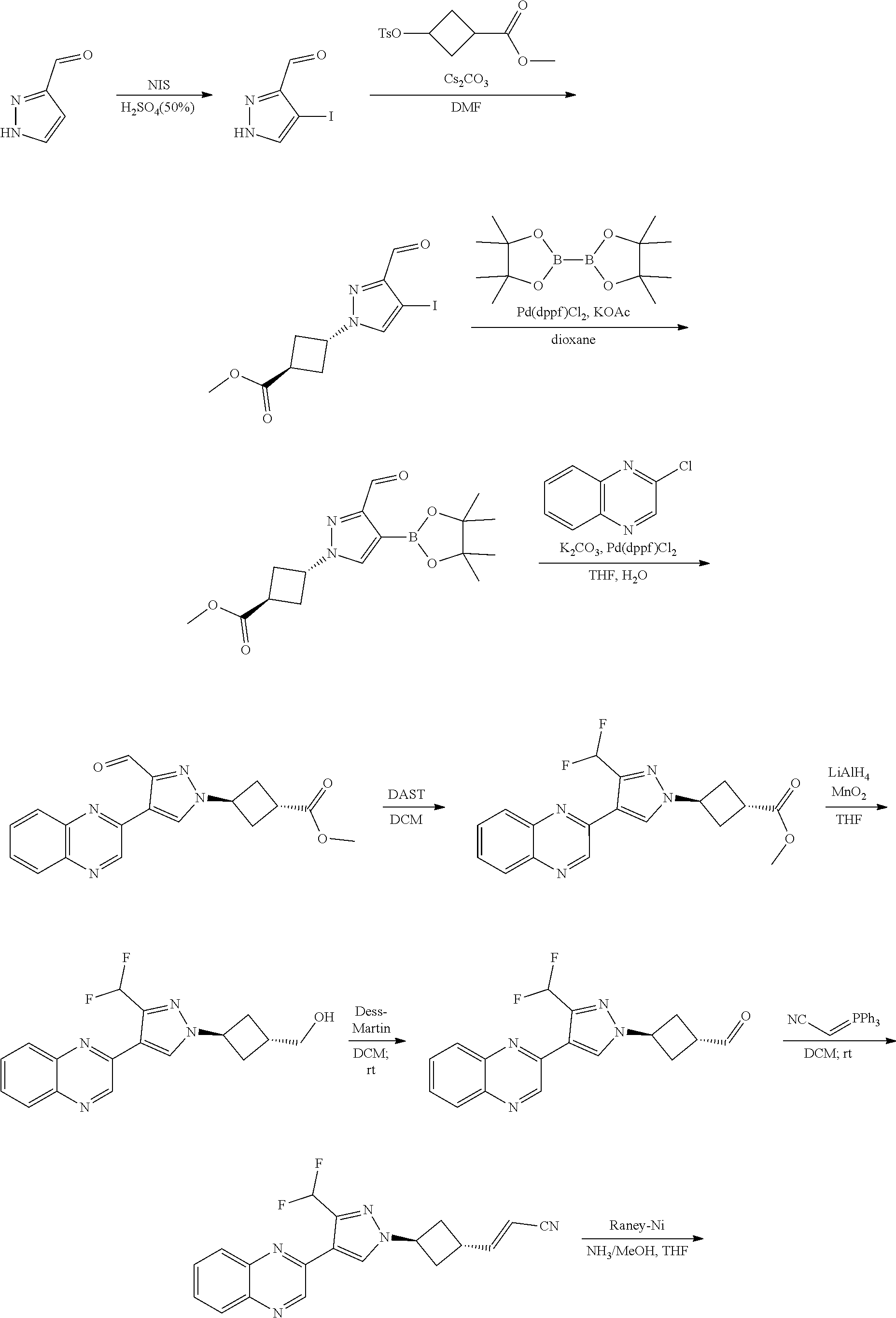

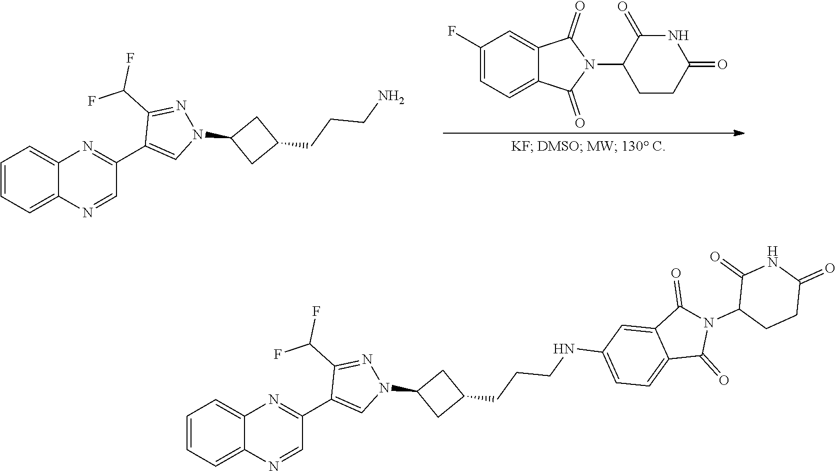 [SMILES: NCCC[C@H]1C[C@H](n2cc(-c3cnc4ccccc4n3)c(C(F)F)n2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccccc7n6)c(C(F)F)n5)C4)cc3C2=O)C(=O)N1]

Step 1. synthesis of 4-iodo-1H-pyrazole-3-carbaldehyde

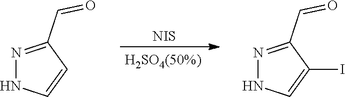

To a solution of 1H-pyrazole-3-carbaldehyde (10 g, 104.1 mmol) in 50% H₂S₄ (80 mL) was added NIS (10.4 g, 104 mmol) at 0° C. The reaction mixture was stirred at rt for 3 h, before it was diluted with H₂O (200 mL) and filtered. The filtrate was washed with H₂Oand dried at 45° C. for 18 h to provide the title compound (17.4 g, 75.7% yield) as a brown solid. MS (ESI) m/z=223.0 [M+H]⁺.

Step 2. Synthesis of methyl trans-3-(3-formyl-4-iodo-5H-pyrazol-1-yl)cyclobutane-1-carboxylate

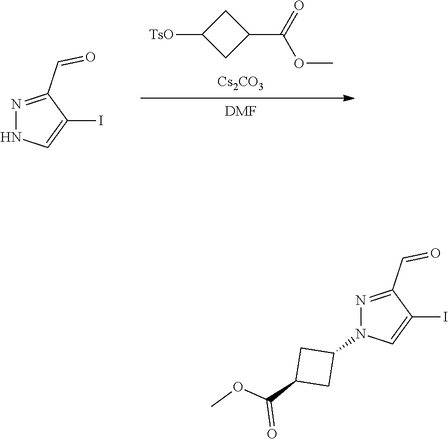

A mixture of 4-iodo-1H-pyrazole-3-carbaldehyde (8 g, 36.2 mmol), methyl 3-(tosyloxy)cyclobutane-1-carboxylate (11.3 g, 39.82 mol) and CS₂CO₃ (35.3 g, 108.6 mmol) in DMF (200 mL) was stirred at 90° C. for 3 h. The reaction mixture was diluted with EtOAc (50 mL), washed with H₂O and brine. The organic layer was concentrated. The resulting residue was purified by prep-TLC to provide the title compound (8.9 g, 66.9% yield) as a pale solid. MS (ESI) m/z=335.1 [M+H]⁺.

Step 3. Synthesis of methyl trans-3-(3-formyl-4-(4,4,5,5-tetramethyl-1,3,2-dioxaborolan-2-yl)-1H-pyrazol-1-yl)cyclobutane-1-carboxylate

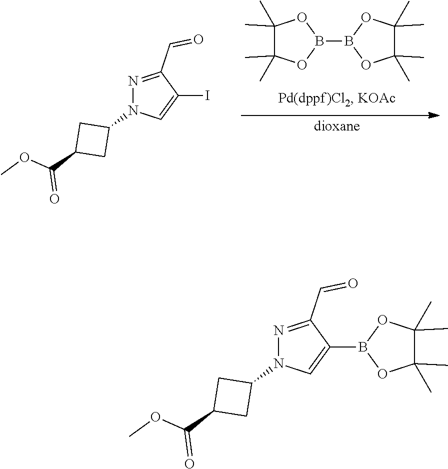

Under nitrogen atmosphere, a mixture of methyl trans-3-(3-formyl-4-iodo-1H-pyrazol-1-yl)cyclobutane-1-carboxylate (8.9 g, 26.6 mmol), 4,4,4′,4′,5,5,5′,5′-octamethyl-2,2′-bi(1,3,2-dioxaborolane) (10.13 g, 39.9 mmol), Pd(dppf)Cl₂ (1.95 g, 2.66 mol) and K₂CO₃ (11.1 g, 79.8 mmol) in dioxone (100 mL) was stirred at 90° C. for 18 h. Then it was diluted with EtOAc, and washed with brine. The organic layer was concentrated. The resulting residue was purified by C18 column chromatography to provide the title compound (4.3 g, 48.3% yield) as a white solid. MS (ESI) m/z=335.2 [M+H]⁺.

Step 4. Synthesis of methyl trans-3-(3-formyl-4-(quinoxalin-2-yl)-1H-pyrazol-1-yl)cyclobutane-1-carboxylate

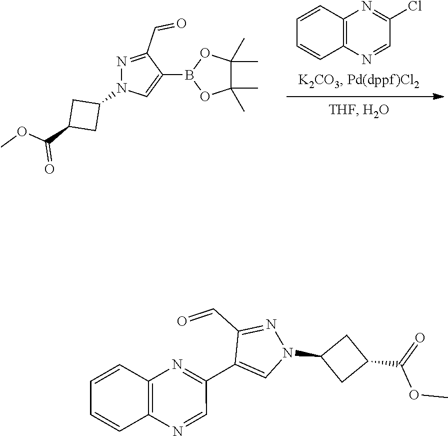

Under nitrogen atmosphere, a mixture of methyl trans-3-(3-formyl-4-(4,4,5,5-tetramethyl-1,3,2-dioxaborolan-2-yl)-1H-pyrazol-1-yl)cyclobutane-1-carboxylate (4.3 g, 12.85 mmol), 2-chloroquinoxaline (2.53 g, 15.42 mmol), Pd(dppf)Cl₂ (945 mg, 1.29 μmol) and K₂CO₃ (5.36 g, 38.55 mmol) in THF (50 mL) and H₂O (5 mL) was stirred at 60° C. for 3 h. Then it was diluted with EtOAc and washed with brine. The organic layer was concentrated. The resulting residue was purified by prep-TLC to provide the title compound (2.5 g, 57.5% yield) as a pale solid. MS (ESI) m/z=337.3 [M+H]⁺.

Step 5. Synthesis of methyl trans-3-(3-(difluoromethyl)-4-(quinoxalin-2-yl)-1H-pyrazol-1-yl)cyclobutane-1-carboxylate

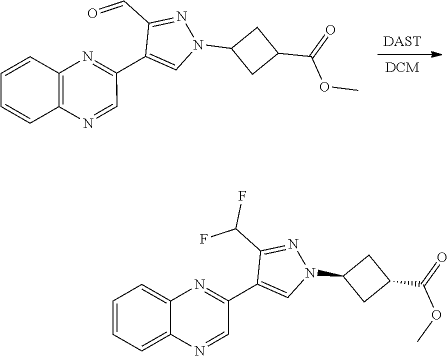

To a solution of methyl trans-3-(3-formyl-4-(quinoxalin-2-yl)-1H-pyrazol-1-yl)cyclobutane-1-carboxylate (2.5 g, 7.4 mmol) in anhydrous DCM (20 mL) at 0° C. was added DAST (11.9 g, 74 mmol). After the reaction was stirred at rt under nitrogen for 4 h, the reaction was diluted with brine. The organic layer was washed with brine, dried over Na₂SO₄, filtered, and concentrated to provide the title compound (545 mg, 20.5% yield) which was used directly in the next step. MS (ESI) m/z=359.4 [M+H]⁺.

Step 6. Synthesis of (trans-3-(3-(difluoromethyl)-4-(quinoxalin-2-yl)-1H-pyrazol-1-yl)cyclobutyl)methanol

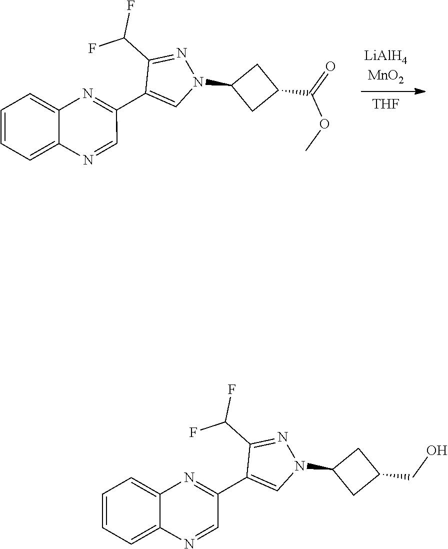 [SMILES: COC(=O)[C@H]1C[C@H](n2cc(-c3cnc4ccccc4n3)c(C(F)F)n2)C1 ; "THF" ; OC[C@H]1C[C@H](n2cc(-c3cnc4ccccc4n3)c(C(F)F)n2)C1]

To a solution of methyl trans-3-(3-(difluoromethyl)-4-(quinoxalin-2-yl)-1H-pyrazol-1-yl)cyclobutane-1-carboxylate (545 mg, 1.52 mmol) in THF (10 mL) was added LiAlH₄ (115 mg, 3.04 mmol) at 0° C. The mixture was stirred at rt for 1 h, before H₂O (0.1 mL) and anhydrous Na₂SO₄ were added. The precipitate was filtered. To the filtrate was added MnO₂ (264.5 mg, 3.04 mmol). The mixture was stirred at rt for 30 min, before it was filtered. The filtrate was concentrated to provide the title compound (360 mg, 71.8% yield) as a colorless oil. MS (ESI) m/z=331.4 [M+H]⁺.

Step 7. Synthesis of trans-3-(3-(difluoromethyl)-4-(quinoxalin-2-yl)-1H-pyrazol-1-yl)cyclobutane-1-carbaldehyde

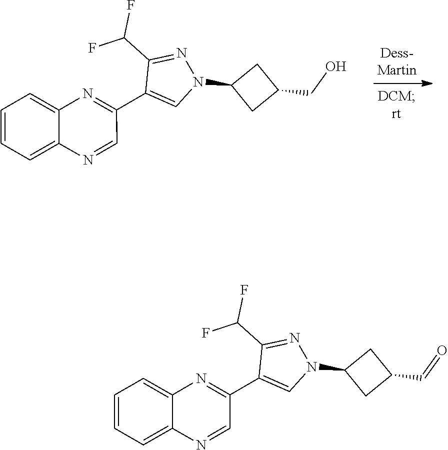 [SMILES: OC[C@H]1C[C@H](n2cc(-c3cnc4ccccc4n3)c(C(F)F)n2)C1 ; O=C[C@H]1C[C@H](n2cc(-c3cnc4ccccc4n3)c(C(F)F)n2)C1]

To a solution of (trans-3-(3-(difluoromethyl)-4-(quinoxalin-2-yl)-1H-pyrazol-1-yl)cyclobutyl)methanol (360 mg, 1.09 mmol) in anhydrous DCM (10 mL) at 0° C. was added Dess-Martin Periodinane (924.3 mg, 2.18 mmol). The reaction was stirred at rt under nitrogen for 1 h, before it was diluted with brine. The organic layer was washed with brine, dried over Na₂SO₄, filtered, and concentrated to provide the title compound (335 mg, 93.4% yield). MS (ESI) m/z=329.1 [M+H]⁺.

Step 8. Synthesis of (E)-3-(trans-3-(3-(difluoromethyl)-4-(quinoxalin-2-yl)-1H-pyrazol-1-yl)cyclobutyl)acrylonitrile

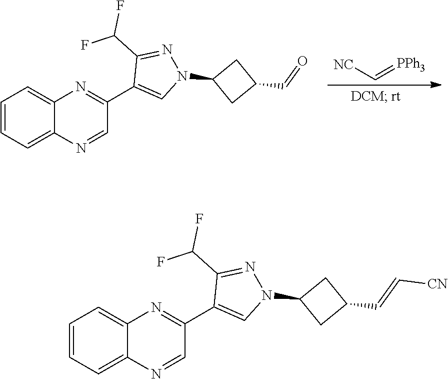

To a solution of trans-3-(3-(difluoromethyl)-4-(quinoxalin-2-yl)-1H-pyrazol-1-yl)cyclobutane-1-carbaldehyde (335 mg, 1.02 mmol) in DCM (10 mL) was added 2-(triphenyl-phosphanylidene)acetonitrile (460 mg, 1.53 mmol) at 0° C. The mixture was stirred at rt for 10 min, before DCM (25 mL) and water (15 mL) were added. The organic phase was washed with water (15 mL), brine (15 mL), dried over Na₂SO₄ and concentrated. The resulting residue was purified by flash chromatography to provide the title compound (280 mg, 77.9% yield) as a white solid. MS (ESI) m/z=352.4 [M+H]⁺.

Step 9. Synthesis of 3-(trans-3-(3-(difluoromethyl)-4-(quinoxalin-2-yl)-1H-pyrazol-1-yl)cyclobutyl)propan-1-amine

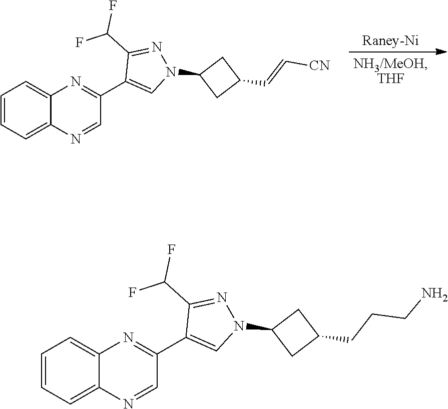 [SMILES: N#C/C=C/[C@H]1C[C@H](n2cc(-c3cnc4ccccc4n3)c(C(F)F)n2)C1 ; "NH3/MeOH,THF" ; "Raney-Ni" ; NCCC[C@H]1C[C@H](n2cc(-c3cnc4ccccc4n3)c(C(F)F)n2)C1]

To a solution of (E)-3-(3-(3-(difluoromethyl)-4-(quinoxalin-2-yl)-1H-pyrazol-1-yl)cyclobutyl)acrylonitrile (280 mg, 794 μmol) in THF (10 mL) and MeOH (10 mL) were added ammonia (0.05 mL) and Raney Ni (50 mg). The reaction mixture was stirred under an atmosphere of hydrogen at rt for 3 h. The reaction mixture was filtered. The filtrate was concentrated to provide the title compound (120 mg, 42.2% yield) as a white solid. MS (ESI) m/z=358.4 [M+H]⁺.

Step 10. Synthesis of 5-((3-(trans-3-(3-(difluoromethyl)-4-(quinoxalin-2-yl)-1H-pyrazol-1-yl)cyclobutyl)propyl)amino)-2-(2,6-dioxopiperidin-3-yl)isoindoline-1,3-dione

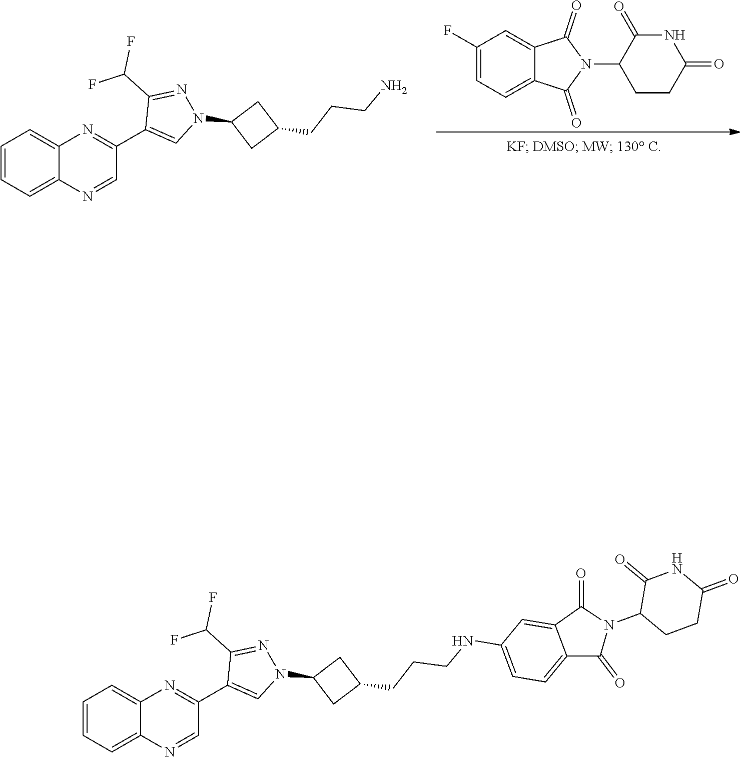

GS-243 was synthesized following the similar procedure for preparing GS-168 (29 mg, yield: 14.1%) MS (ESI) m/z=614.5 [M+H]⁺.

### Example 202. 2-(2,6-Dioxopiperidin-3-yl)-5-((3-(trans-3-(3-methyl-4-(6-morpholinopyridin-2-yl)-1H-pyrazol-1-yl)cyclobutyl)propyl)amino)isoindoline-1,3-dione (GS-244)

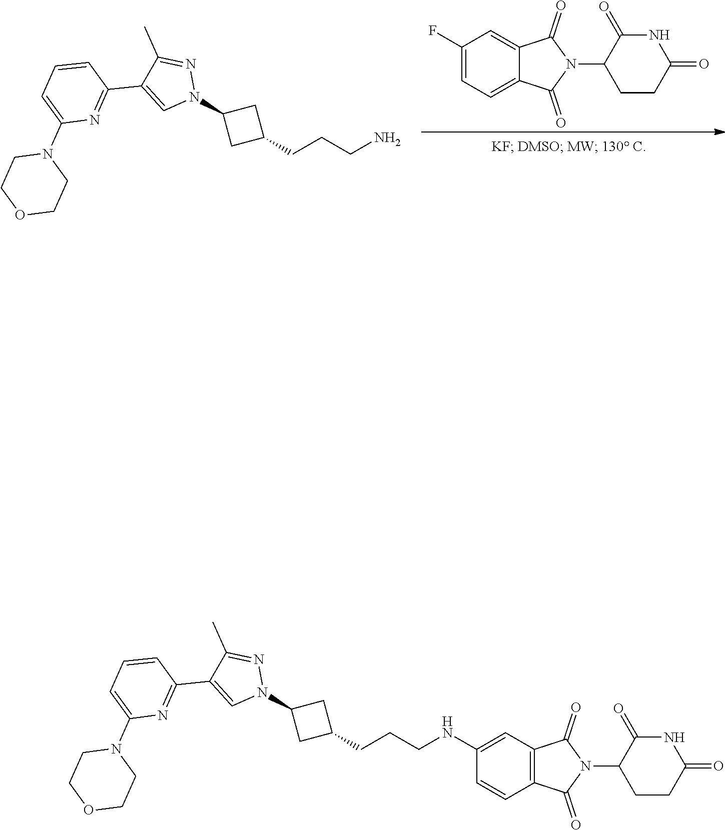

GS-244 was synthesized following the similar procedure for preparing GS-168 (2.6 mg, yield: 3.8%). MS (ESI) m/z=612.6 [M+H]⁺.

### Example 203. 2-(2,6-Dioxopiperidin-3-yl)-5-((3-(trans-3-(4-(6-(4-methylpiperazin-1-yl)pyridin-2-yl)-1H-pyrazol-1-yl)cyclobutyl)propyl)amino)isoindoline-1,3-dione (GS-245)

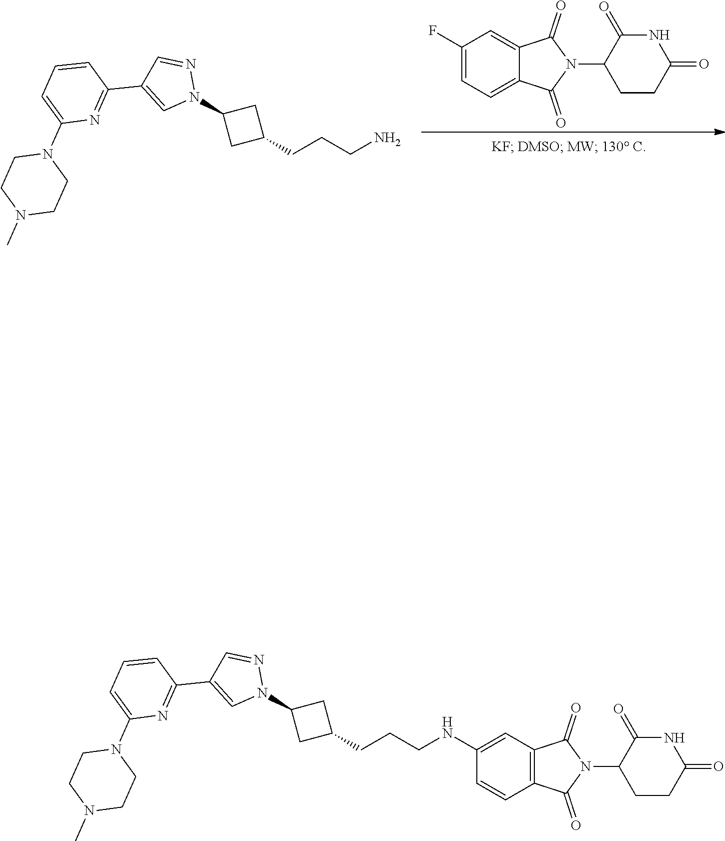

GS-245 was synthesized following the similar procedure for preparing GS-168 (5.6 mg, yield: 6.9%). MS (ESI) m/z=611.6 [M+H]⁺.

### Example 204. 5-((3-(trans-3-(3-Cyclopropyl-4-(6-morpholinopyridin-2-yl)-1H-pyrazol-1-yl)cyclobutyl)propyl)amino)-2-(2,6-dioxopiperidin-3-yl)isoindoline-1,3-dione (GS-246)

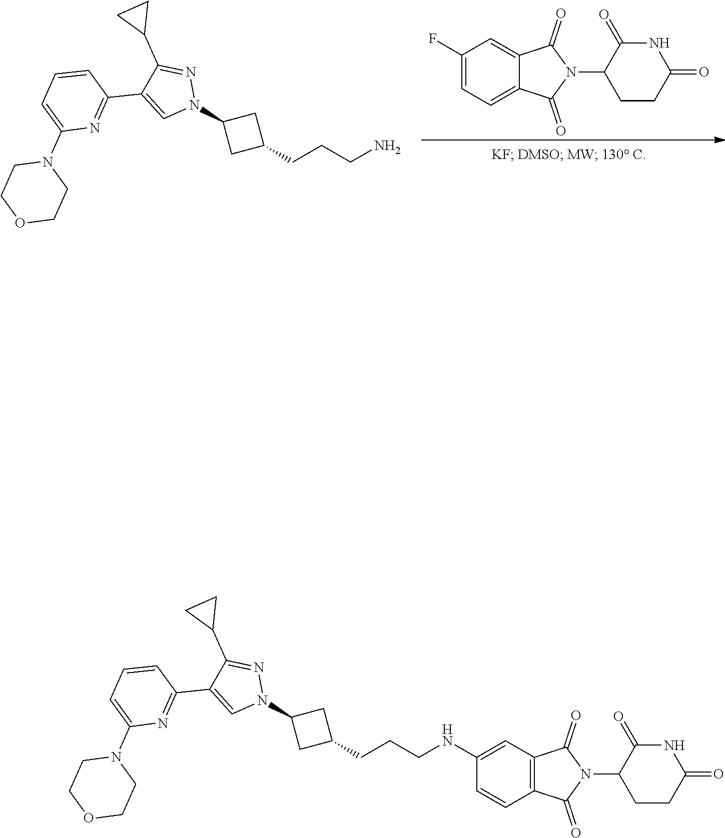

GS-246 was synthesized following the similar procedure for preparing GS-168 (2.4 mg, yield: 5.2%). MS (ESI) m/z=638.7 [M+H]⁺.

### Example 205. 2-(2,6-Dioxopiperidin-3-yl)-5-((3-(trans-3-(4-(6-(piperazin-1-yl)pyridin-2-yl)-1H-pyrazol-1-yl)cyclobutyl)propyl)amino)isoindoline-1,3-dione (GS-247)

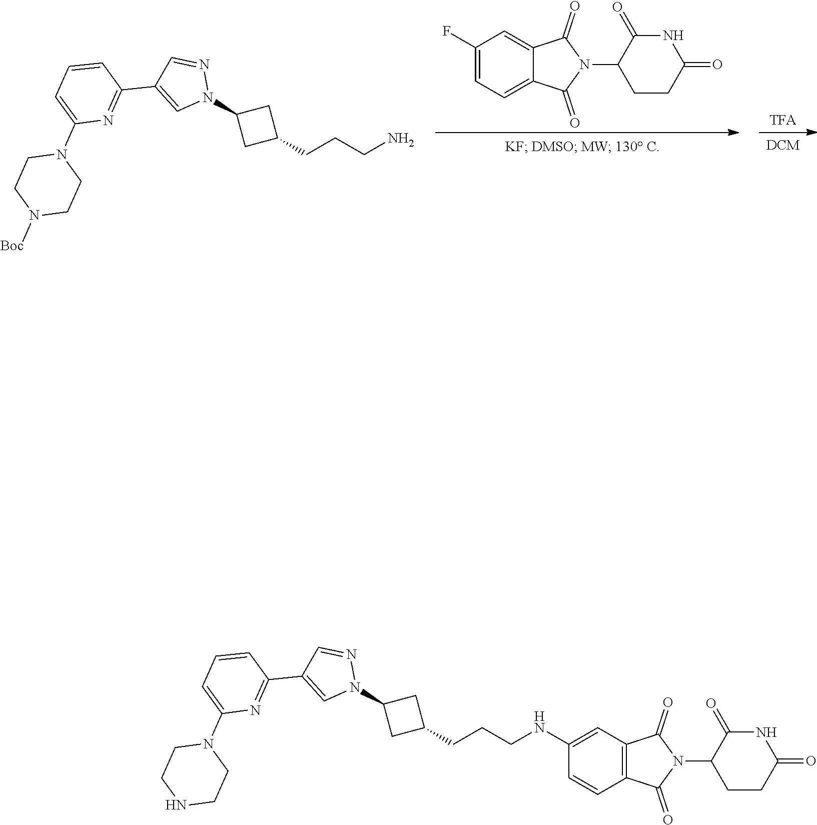

GS-247 was synthesized following the similar procedure for preparing GS-174 (17 mg, yield: 22.9%). MS (ESI) m/z=597.8 [M+H]⁺.

### Example 206. 5-((3-(trans-3-(4-(6-(Dimethylamino)pyridin-2-yl)-1H-pyrazol-1-yl)cyclobutyl)propyl)amino)-2-(2,6-dioxopiperidin-3-yl)isoindoline-1,3-dione (GS-248)

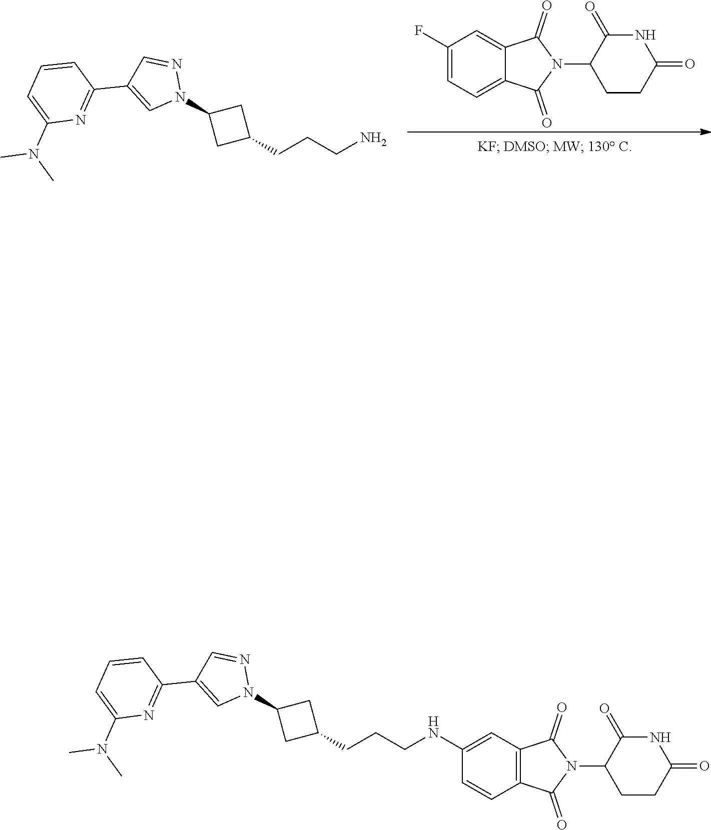

GS-248 was synthesized following the similar procedure for preparing GS-168 (14.7 mg, yield: 32.9%). MS (ESI) m/z=556.6 [M+H]⁺.

### Example 207. 2-(2,6-Dioxopiperidin-3-yl)-5-((3-(trans-3-(4-(6-oxo-1,6-dihydropyridin-2-yl)-1H-pyrazol-1-yl)cyclobutyl)propyl)amino)isoindoline-1,3-dione (GS-249)

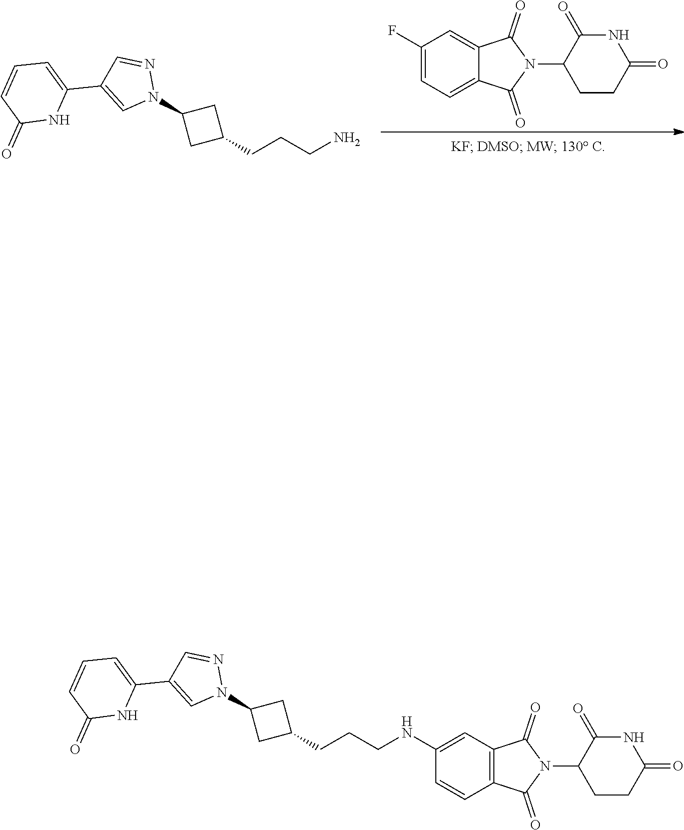

GS-249 was synthesized following the similar procedure for preparing GS-168 (8 mg, yield: 27.5%). MS (ESI) m/z=529.6 [M+H]⁺.

### Example 208. 5-((3-(trans-3-(4-(5-(Azetidin-3-yl)quinoxalin-2-yl)-1H-pyrazol-1-yl)cyclobutyl)propyl)amino)-2-(2,6-dioxopiperidin-3-yl)isoindoline-1,3-dione (GS-250)

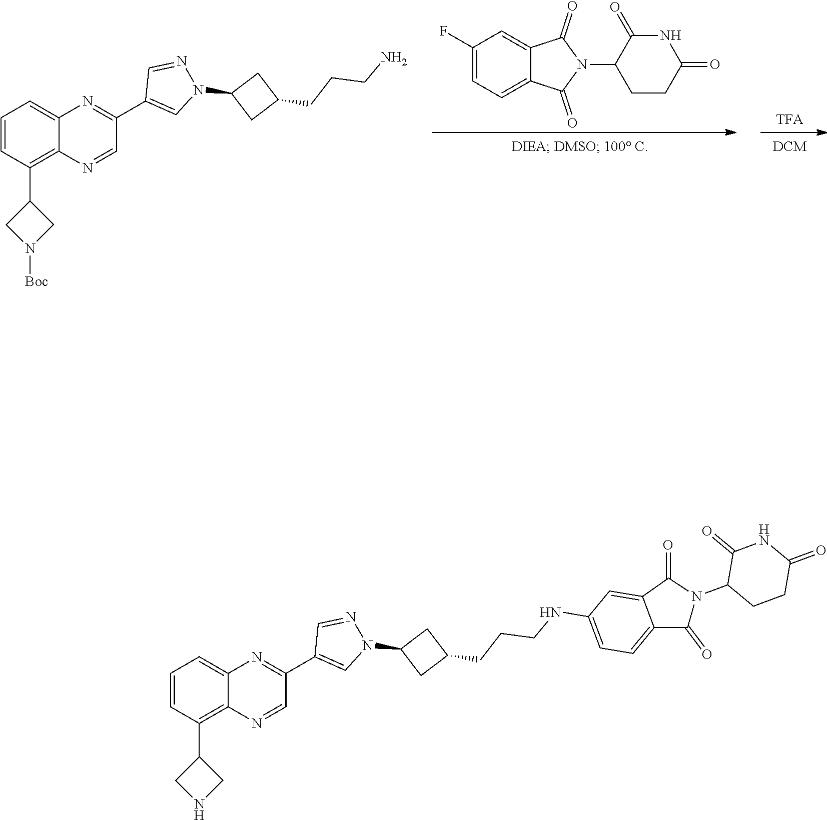

GS-250 was synthesized following the similar procedure for preparing GS-174 (3.6 mg, yield: 5.2%). MS (ESI) m/z=619.7 [M+H]⁺.

### Example 209. 2-(2,6-Dioxopiperidin-3-yl)-5-((3-(trans-3-(4-(6-(methylamino)pyridin-2-yl)-1H-pyrazol-1-yl)cyclobutyl)propyl)amino)isoindoline-1,3-dione (GS-251)

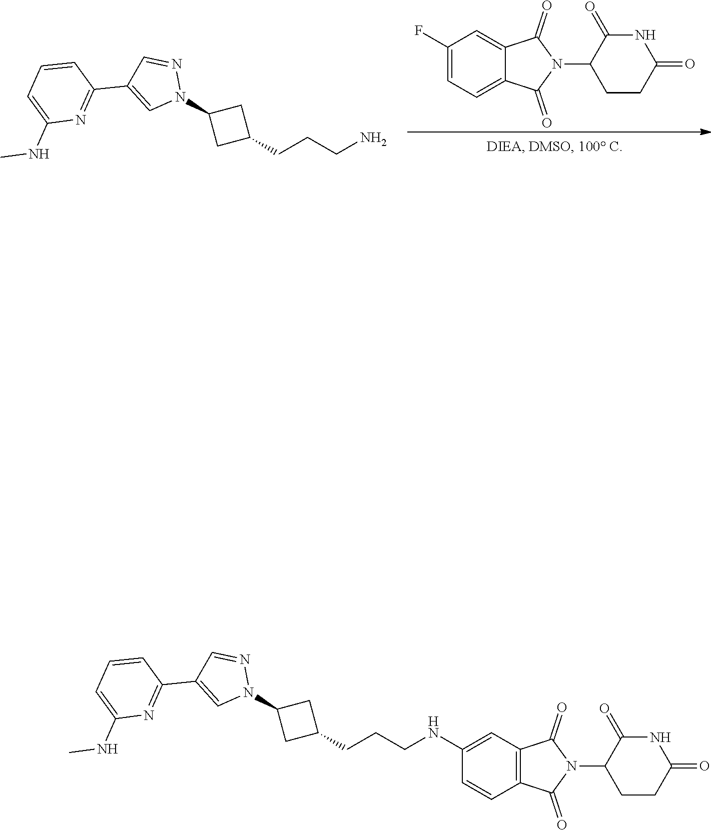

GS-251 was synthesized following the similar procedure for preparing GS-168 (1.4 mg, yield: 4.3%). MS (ESI) m/z=542.6 [M+H]⁺.

### Example 210. 2-(2,6-Dioxopiperidin-3-yl)-5-((3-(trans-3-(4-(6-(4-hydroxypiperidin-1-yl)pyridin-2-yl)-1H-pyrazol-1-yl)cyclobutyl)propyl)amino)isoindoline-1,3-dione (GS-252)

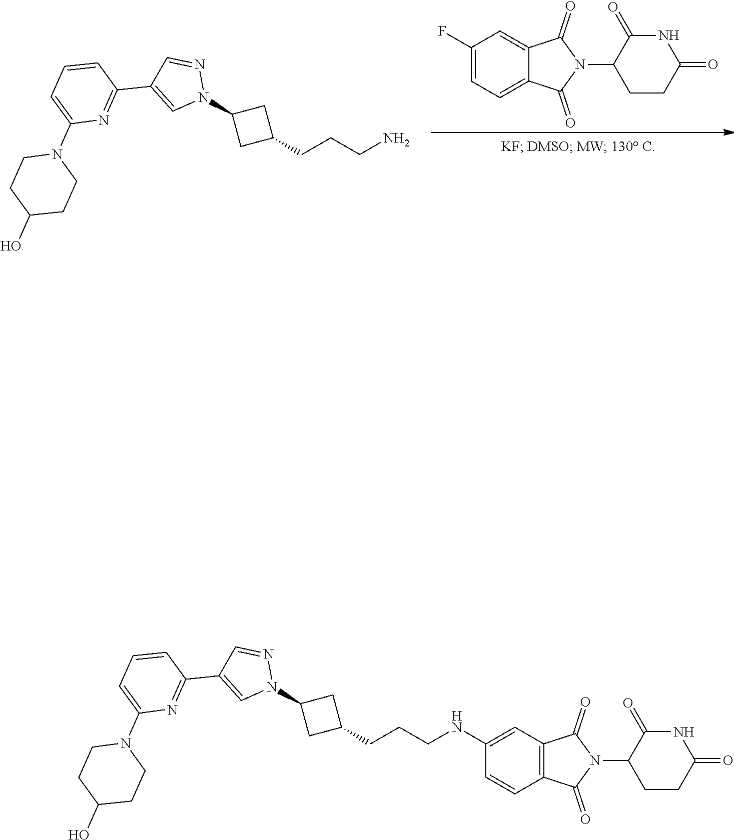

GS-252 was synthesized following the similar procedure for preparing GS-168 (1.1 mg, yield: 1.9%). MS (ESI) m/z=612.6 [M+H]⁺.

### Example 211. 5-(((trans-3-(3-Cyclopropyl-4-(4-methylpyridin-2-yl)-1H-pyrazol-1-yl)cyclobutyl)methyl)amino)-2-(2,6-dioxopiperidin-3-yl)isoindoline-1,3-dione (GS-253)

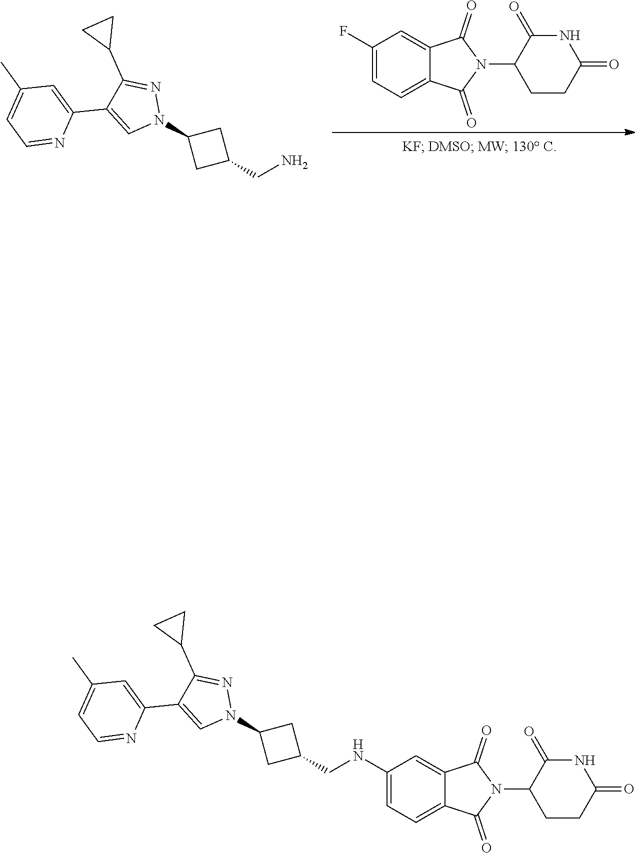

GS-253 was synthesized following the similar procedure for preparing GS-178 (78.1 mg, yield: 51.2%). MS (ESI) m/z=539.5 [M+H]⁺.

### Example 212. 5-(((trans-3-(3-Cyclopropyl-4-(4-methoxypyridin-2-yl)-1H-pyrazol-1-yl)cyclobutyl)methyl)amino)-2-(2,6-dioxopiperidin-3-yl)isoindoline-1,3-dione (GS-254)

GS-254 was synthesized following the similar procedure for preparing GS-178 (53 mg, yield: 31.7%). MS (ESI) m/z=555.5 [M+H]⁺.

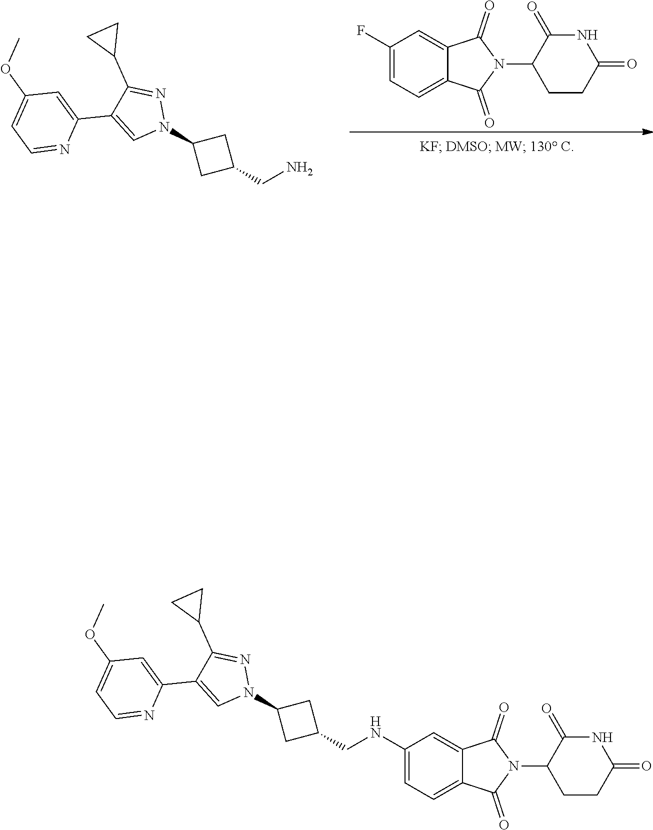

### Example 213. 5-(((trans-3-(4-(5-Chloropyridin-2-yl)-3-cyclopropyl-1H-pyrazol-1-yl)cyclobutyl)methyl)amino)-2-(2,6-dioxopiperidin-3-yl)isoindoline-1,3-dione (GS-255)

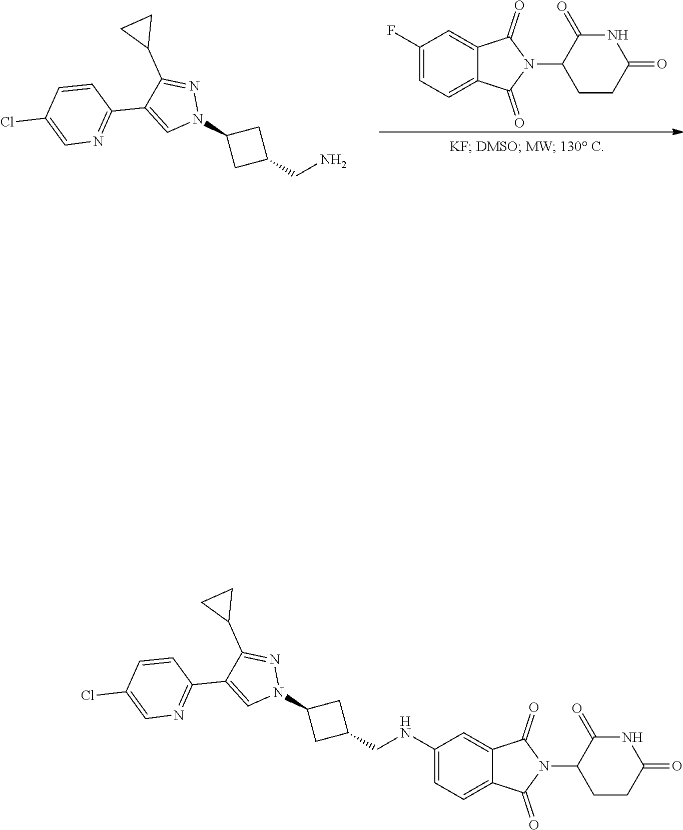

GS-255 was synthesized following the similar procedure for preparing GS-178 (8.4 mg, yield: 22.6%). MS (ESI) m/z=559.5 [M+H]⁺.

### Example 214. 5-(((trans-3-(3-Cyclopropyl-4-(5-methylpyridin-2-yl)-1H-pyrazol-1-yl)cyclobutyl)methyl)amino)-2-(2,6-dioxopiperidin-3-yl)isoindoline-1,3-dione (GS-256)

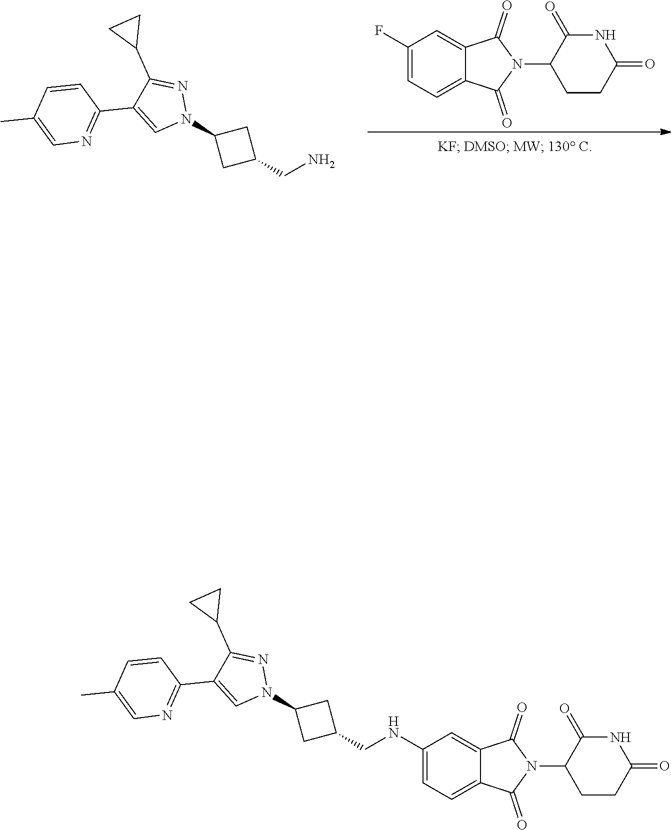

GS-256 was synthesized following the similar procedure for preparing GS-178 (26.7 mg, yield: 27.9%). MS (ESI) m/z=539.7 [M+H]⁺.

### Example 215. 5-(((trans-3-(4-(6-(Azetidin-1-yl)pyridin-2-yl)-3-cyclopropyl-1H-pyrazol-1-yl)cyclobutyl)methyl)amino)-2-(2,6-dioxopiperidin-3-yl)isoindoline-1,3-dione (GS-257)

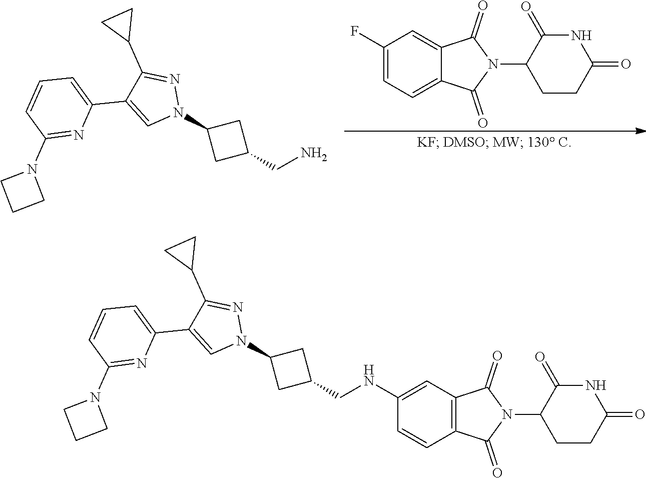 [SMILES: NC[C@H]1C[C@H](n2cc(-c3cccc(N4CCC4)n3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cccc(N7CCC7)n6)c(C6CC6)n5)C4)cc3C2=O)C(=O)N1]

GS-257 was synthesized following the similar procedure for preparing GS-178 (2.3 mg, yield: 3.9%). MS (ESI) m/z=580.6 [M+H]⁺.

### Example 216. 5-(((trans-3-(4-(6-(Cyclobutylamino)pyridin-2-yl)-3-cyclopropyl-1H-pyrazol-1-yl)cyclobutyl)methyl)amino)-2-(2,6-dioxopiperidin-3-yl)isoindoline-1,3-dione (GS-258)

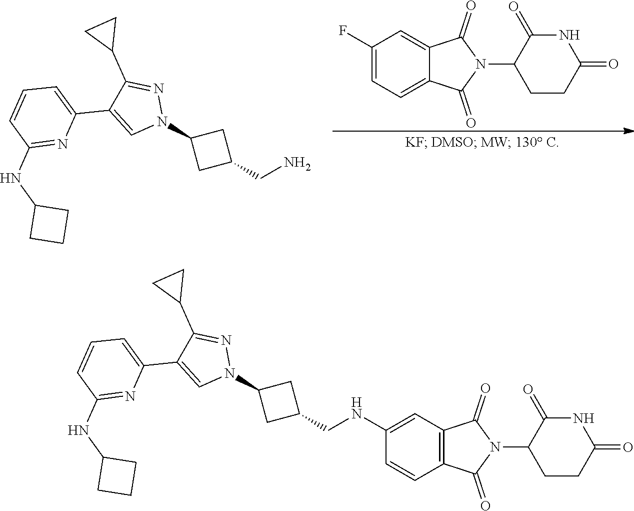 [SMILES: NC[C@H]1C[C@H](n2cc(-c3cccc(NC4CCC4)n3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cccc(NC7CCC7)n6)c(C6CC6)n5)C4)cc3C2=O)C(=O)N1]

GS-258 was synthesized following the similar procedure for preparing GS-178 (2.2 mg, yield: 2.7%). MS (ESI) m/z=594.6 [M+H]⁺.

### Example 217. 5-(((trans-3-(3-Cyclopropyl-4-(6-(dimethylamino)pyridin-2-yl)-1H-pyrazol-1-yl)cyclobutyl)methyl)amino)-2-(2,6-dioxopiperidin-3-yl)isoindoline-1,3-dione (GS-259)

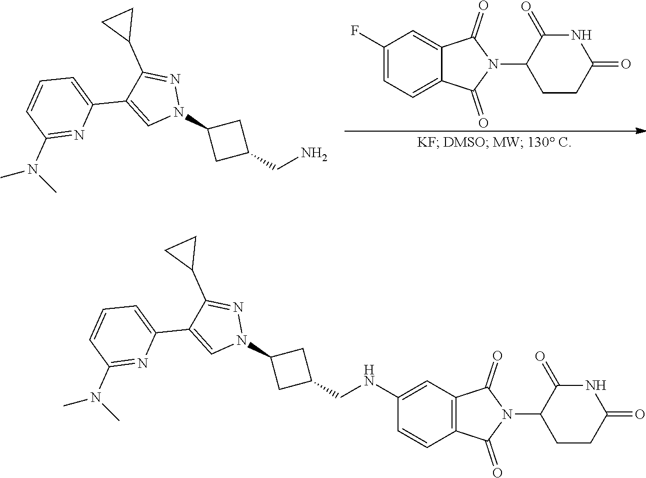 [SMILES: CN(C)c1cccc(-c2cn([C@H]3C[C@H](CN)C3)nc2C2CC2)n1 ; O=C1CCC(N2C(=O)c3ccc(F)cc3C2=O)C(=O)N1 ; CN(C)c1cccc(-c2cn([C@H]3C[C@H](CNc4ccc5c(c4)C(=O)N(C4CCC(=O)NC4=O)C5=O)C3)nc2C2CC2)n1]

GS-259 was synthesized following the similar procedure for preparing GS-178 (1.8 mg, yield: 3.3%). MS (ESI) m/z=568.6 [M+H]⁺.

### Example 218. 5-(((trans-3-(3-Cyclopropyl-4-(6-(oxetan-3-ylamino)pyridin-2-yl)-1H-pyrazol-1-yl)cyclobutyl)methyl)amino)-2-(2,6-dioxopiperidin-3-yl)isoindoline-1,3-dione (GS-260)

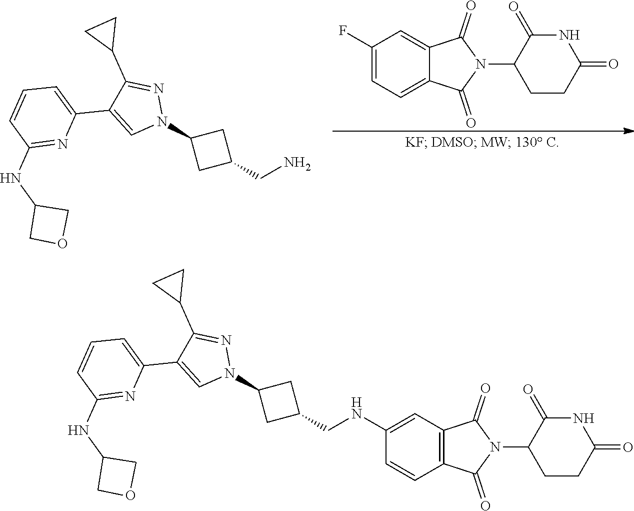 [SMILES: NC[C@H]1C[C@H](n2cc(-c3cccc(NC4COC4)n3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cccc(NC7COC7)n6)c(C6CC6)n5)C4)cc3C2=O)C(=O)N1]

GS-260 was synthesized following the similar procedure for preparing GS-178 (5.8 mg, yield: 9.2%). MS (ESI) m/z=596.6 [M+H]⁺.

### Example 219. 5-(((trans-3-(3-Cyclopropyl-4-(3-methylpyridin-2-yl)-1H-pyrazol-1-yl)cyclobutyl)methyl)amino)-2-(2,6-dioxopiperidin-3-yl)isoindoline-1,3-dione (GS-261)

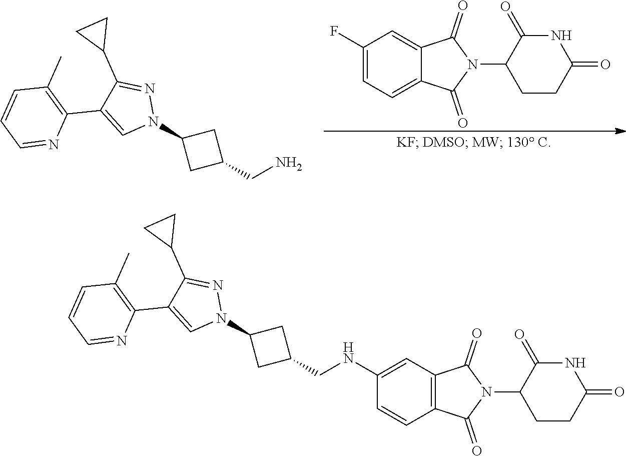

GS-261 was synthesized following the similar procedure for preparing GS-178 (35 mg, yield: 21%). MS (ESI) m/z=539.8 [M+H]⁺.

### Example 220. 5-(((trans-3-(3-Cyclopropyl-4-(3-methoxypyridin-2-yl)-1H-pyrazol-1-yl)cyclobutyl)methyl)amino)-2-(2,6-dioxopiperidin-3-yl)isoindoline-1,3-dione (GS-262)

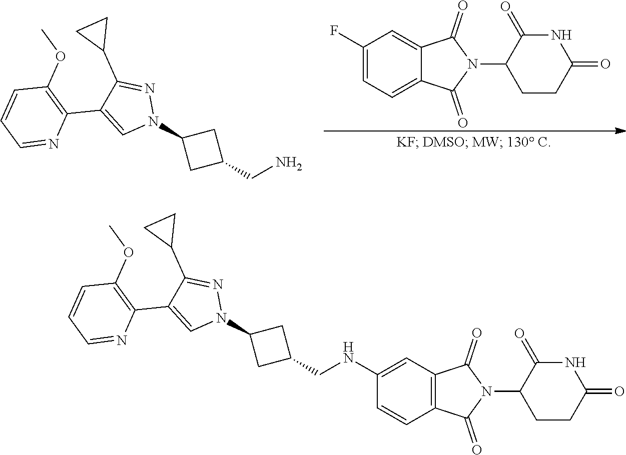

GS-262 was synthesized following the similar procedure for preparing GS-178 (120 mg, yield: 19.6%). MS (ESI) m/z=555.8 [M+H]⁺.

### Example 221. 5-(((trans-3-(3-Cyclopropyl-4-(6-(methylamino)pyridin-2-yl)-1H-pyrazol-1-yl)cyclobutyl)methyl)amino)-2-(2,6-dioxopiperidin-3-yl)isoindoline-1,3-dione (GS-263)

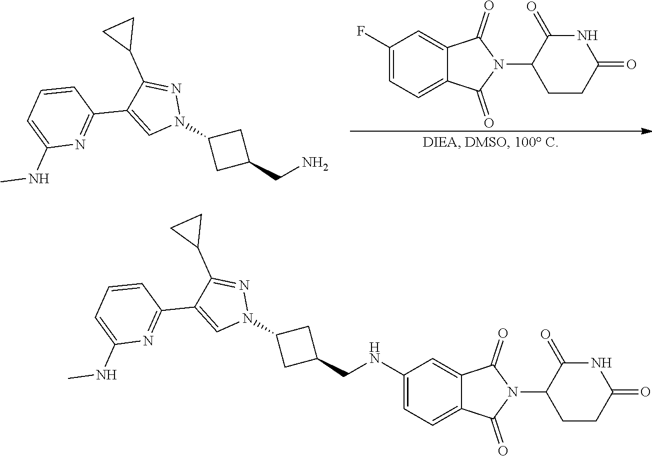

GS-263 was synthesized following the similar procedure for preparing GS-178 (3.2 mg, yield: 9.7%). MS (ESI) m/z=554.6 [M+H]⁺.

### Example 222. 5-(((trans-3-(4-(4-Chloropyridin-2-yl)-3-cyclopropyl-1H-pyrazol-1-yl)cyclobutyl)methyl)amino)-2-(2,6-dioxopiperidin-3-yl)isoindoline-1,3-dione (GS-264)

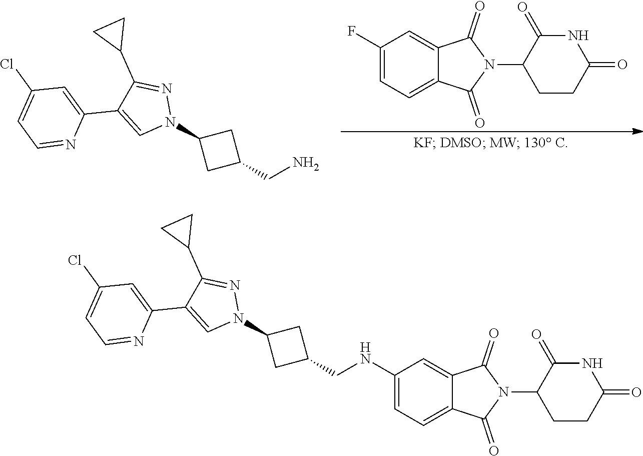

GS-264 was synthesized following the similar procedure for preparing GS-178 (60 mg, yield: 35.6%). MS (ESI) m/z=559.7 [M+H]⁺.

### Example 223. 5-(((trans-3-(3-Cyclopropyl-4-(pyridin-4-yl)-1H-pyrazol-1-yl)cyclobutyl)methyl)amino)-2-(2,6-dioxopiperidin-3-yl)isoindoline-1,3-dione (GS-265)

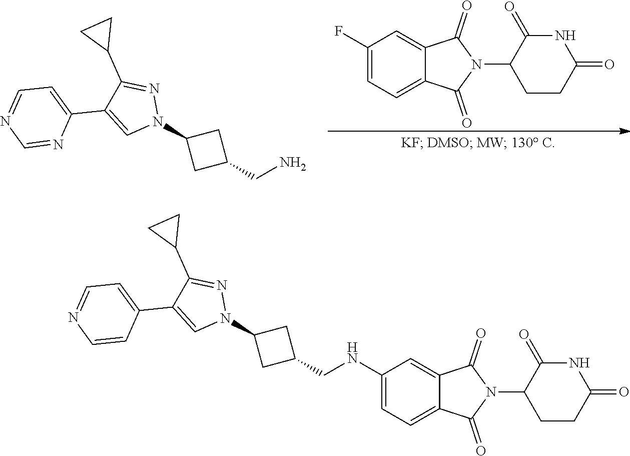

GS-265 was synthesized following the similar procedure for preparing GS-178 (11.7 mg, yield: 25.8%). MS (ESI) m/z=525.7 [M+H]⁺.

### Example 224. 5-(((trans-3-(3-Cyclopropyl-4-(5-morpholinoquinoxalin-2-yl)-1H-pyrazol-1-yl)cyclobutyl)methyl)amino)-2-(2,6-dioxopiperidin-3-yl)isoindoline-1,3-dione (GS-266)

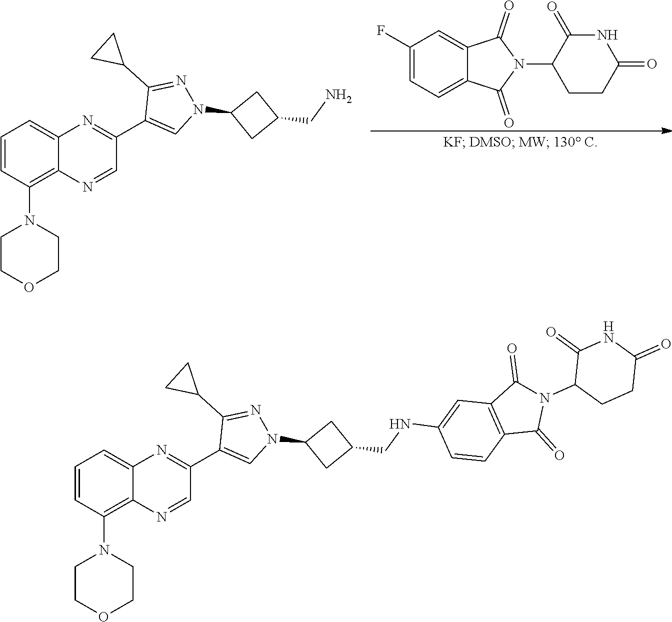 [SMILES: NC[C@H]1C[C@H](n2cc(-c3cnc4c(N5CCOCC5)cccc4n3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cnc7c(N8CCOCC8)cccc7n6)c(C6CC6)n5)C4)cc3C2=O)C(=O)N1]

GS-266 was synthesized following the similar procedure for preparing GS-178 (60 mg, yield: 19.6%). MS (ESI) m/z=661.7 [M+H]⁺.

### Example 225. 5-(((trans-3-(3-Cyclopropyl-4-(5-(4-methylpiperazin-1-yl)quinoxalin-2-yl)-1H-pyrazol-1-yl)cyclobutyl)methyl)amino)-2-(2,6-dioxopiperidin-3-yl)isoindoline-1,3-dione (GS-267)

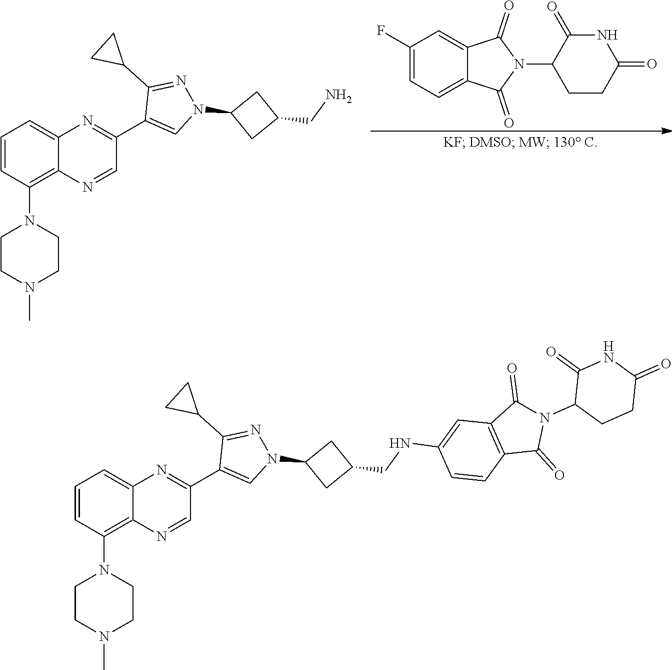 [SMILES: CN1CCN(c2cccc3nc(-c4cn([C@H]5C[C@H](CN)C5)nc4C4CC4)cnc23)CC1 ; O=C1CCC(N2C(=O)c3ccc(F)cc3C2=O)C(=O)N1 ; CN1CCN(c2cccc3nc(-c4cn([C@H]5C[C@H](CNc6ccc7c(c6)C(=O)N(C6CCC(=O)NC6=O)C7=O)C5)nc4C4CC4)cnc23)CC1]

GS-267 was synthesized following the similar procedure for preparing GS-178 (98 mg, yield: 25.7%). MS (ESI) m/z=674.8 [M+H]⁺.

### Example 226. 5-(((trans-3-(3-Cyclopropyl-4-(pyridin-3-yl)-1H-pyrazol-1-yl)cyclobutyl)methyl)amino)-2-(2,6-dioxopiperidin-3-yl)isoindoline-1,3-dione (GS-268)

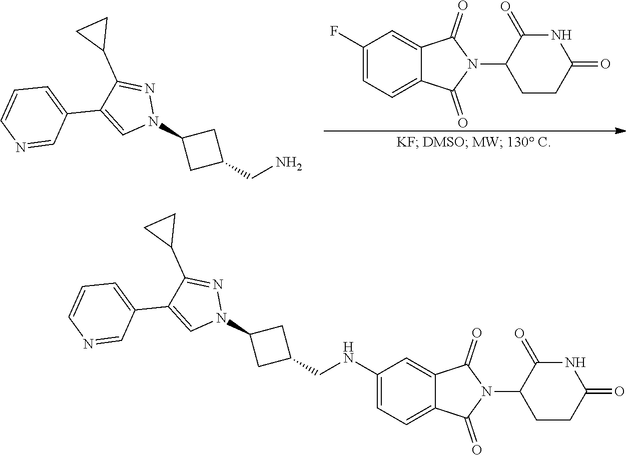

GS-268 was synthesized following the similar procedure for preparing GS-178 (50 mg, yield: 30.4%). MS (ESI) m/z=525.8 [M+H]⁺.

### Example 227. 5-(((trans-3-(3-Cyclopropyl-4-(6-((3-hydroxycyclobutyl)amino)pyridin-2-yl)-1H-pyrazol-1-yl)cyclobutyl)methyl)amino)-2-(2,6-dioxopiperidin-3-yl)isoindoline-1,3-dione (GS-269)

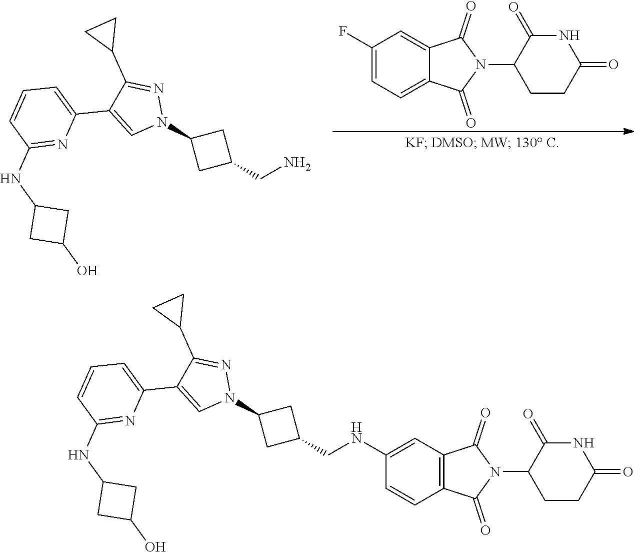 [SMILES: NC[C@H]1C[C@H](n2cc(-c3cccc(NC4CC(O)C4)n3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cccc(NC7CC(O)C7)n6)c(C6CC6)n5)C4)cc3C2=O)C(=O)N1]

GS-269 was synthesized following the similar procedure for preparing GS-178 (3.6 mg, yield: 4.6%). MS (ESI) m/z=610.7 [M+H]⁺.

### Example 228. 5-(((trans-3-(3-Cyclopropyl-4-(6-oxo-1,6-dihydropyridin-2-yl)-1H-pyrazol-1-yl)cyclobutyl)methyl)amino)-2-(2,6-dioxopiperidin-3-yl)isoindoline-1,3-dione (GS-270)

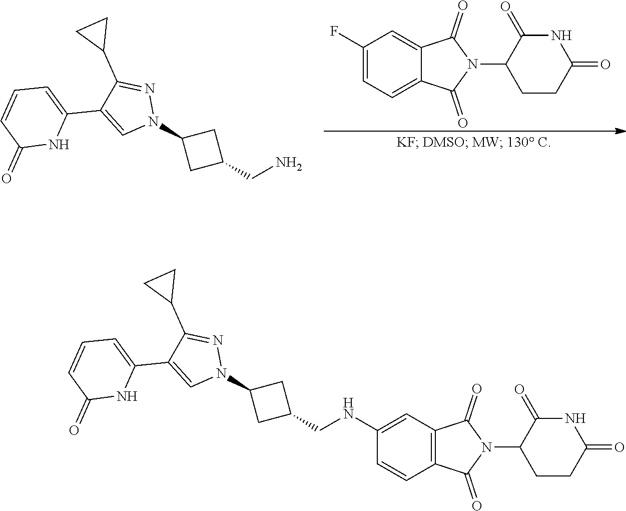 [SMILES: NC[C@H]1C[C@H](n2cc(-c3cccc(=O)[nH]3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cccc(=O)[nH]6)c(C6CC6)n5)C4)cc3C2=O)C(=O)N1]

GS-270 was synthesized following the similar procedure for preparing GS-178 (1.9 mg, yield: 4.4%). MS (ESI) m/z=541.6 [M+H]⁺.

### Example 229. 2-(3-Cyclopropyl-4-(quinoxalin-2-yl)-1H-pyrazol-1-yl)-N-((2-(2,6-dioxopiperidin-3-yl)-1,3-dioxoisoindolin-5-yl)methyl)acetamide (GS-271)

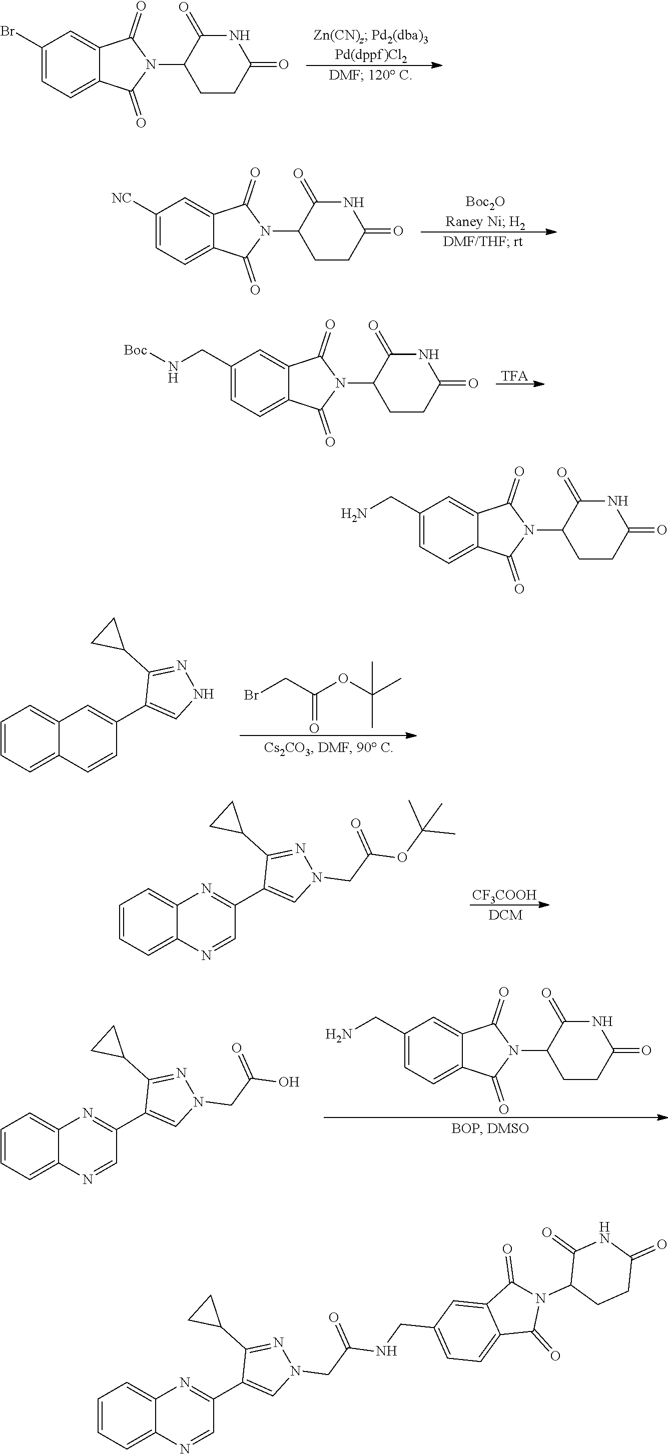

Step 1. Synthesis of 2-(2,6-dioxopiperidin-3-yl)-1,3-dioxoisoindoline-5-carbonitrile

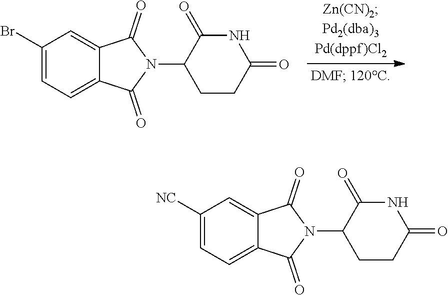 [SMILES: O=C1CCC(N2C(=O)c3ccc(Br)cc3C2=O)C(=O)N1 ; N#Cc1ccc2c(c1)C(=O)N(C1CCC(=O)NC1=O)C2=O]

A mixture of 5-bromo-2-(2,6-dioxo-3-piperidyl)isoindoline-1,3-dione (2 g, 5.93 mmol), Zn(CN)₂ (417.97 mg, 3.56 mmol), Pd₂(dba)₃ (108.65 mg, 118.65 μmol) and Pd(dppf)Cl₂ (173.63 mg, 237.30 μmol) in DMF (43 mL) was stirred at 120° C. for 4 h. After being cooled to 60° C., the mixture was filtered through Celite, and washed with DMF (160 mL). The filtrate was concentrated. The resulting residue was stirred in water (300 mL) for 2 h, before it was filtered, washed with water, dried under vacuum to provide the tilte compound (1.6 g, 95.2% yield) as an off-white solid. MS (ESI) m/z=284.1 [M+H]⁺.

Step 2. Synthesis of tert-butyl ((2-(2,6-dioxopiperidin-3-yl)-1,3-dioxoisoindolin-5-yl)methyl)carbamate

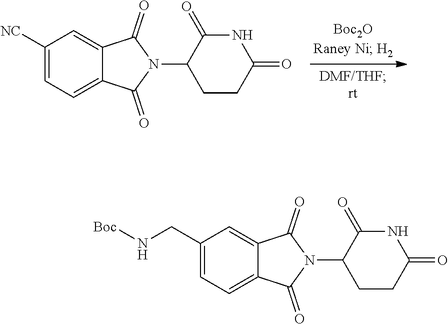 [SMILES: N#Cc1ccc2c(c1)C(=O)N(C1CCC(=O)NC1=O)C2=O ; CC(C)(C)OC(=O)NCc1ccc2c(c1)C(=O)N(C1CCC(=O)NC1=O)C2=O]

To a solution of 2-(2,6-dioxo-3-piperidyl)-1,3-dioxo-isoindoline-5-carbonitrile (300 mg, 1.06 mmol) and Boc₂O (369.86 mg, 1.69 mmol) in DMF (5 mL) and THF (10 mL) was added Raney-Nickel (621.63 mg, 10.59 mmol). The resulting mixture was stirred at rt for 14 h under H₂ atmosphere. The mixture was filtered. The filtrate was diluted with EtOAc (25 mL) and H₂O (30 mL). The organic phase was separated, washed with brine, dried over Na₂SO₄, and concentrated to provide the title compound (320 mg, 78% yield) as an off-white solid. MS (ESI) m/z=388.3 [M+H]⁺.

Step 3. Synthesis of 5-(aminomethyl)-2-(2,6-dioxopiperidin-3-yl)isoindoline-1,3-dione

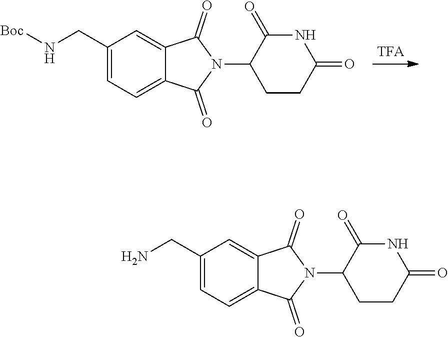 [SMILES: CC(C)(C)OC(=O)NCc1ccc2c(c1)C(=O)N(C1CCC(=O)NC1=O)C2=O ; NCc1ccc2c(c1)C(=O)N(C1CCC(=O)NC1=O)C2=O]

To a solution of tert-butyl ((2-(2,6-dioxopiperidin-3-yl)-1,3-dioxoisoindolin-5-yl)methyl)carbamate (330 mg, 851.86 μmol) in DCM (4 mL) was added TFA (1.94 g, 17.04 mmol). The mixture was stirred at rt for 30 min, before it was concentrated to provide the title compound (341.8 mg, 99% yield) as a brown oil. MS (ESI) m/z=288.3 [M+H]⁺.

Step 4. Synthesis of tert-butyl 2-(3-cyclopropyl-4-(quinoxalin-2-yl)-1H-pyrazol-1-yl)acetate

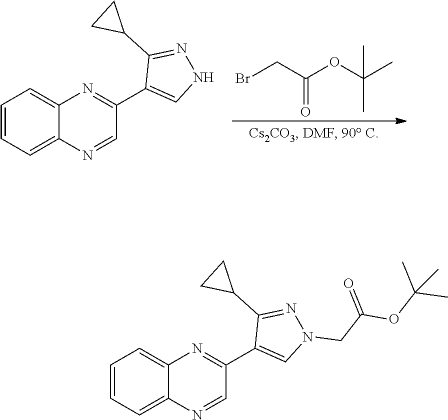

A mixture of 2-(3-cyclopropyl-1H-pyrazol-4-yl)quinoxaline (450 mg, 1.88 mmol), tert-butyl 2-bromoacetate (734 mg, 3.76 mmol) and Cs₂CO₃ (1.83 g, 5.64 mmol) in DMF (15 mL) was stirred at 90° C. for 2.5 h. The reaction mixture was diluted with EtOAc (250 mL), washed with H₂O (200 mL) and brine (250 mL). The organic layer was concentrated. The resulting residue was purified by prep-HPLC to provide the title compound (350 mg, 53.2% yield) as a yellow solid. MS (ESI) m/z=351.4 [M+H]⁺.

Step 5. Synthesis of 2-(3-cyclopropyl-4-(quinoxalin-2-yl)-1H-pyrazol-1-yl)acetic acid

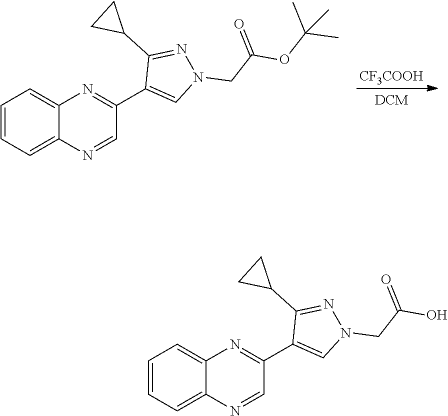 [SMILES: CC(C)(C)OC(=O)Cn1cc(-c2cnc3ccccc3n2)c(C2CC2)n1 ; "CF3COOH" ; O=C(O)Cn1cc(-c2cnc3ccccc3n2)c(C2CC2)n1]

To a solution of tert-butyl 2-(3-cyclopropyl-4-(quinoxalin-3-yl)-1H-pyrazol-1-yl)acetate (50 mg, 0.149 mmol) in DCM (5.0 mL) was added TFA (5 mL). The mixture was stirred at rt for 2.5 h, before it was concentrated to provide the crude product which was used directly in the next step. MS (ESI) m/z=295.2 [M+H]⁺.

Step 6. Synthesis of 2-(3-cyclopropyl-4-(quinoxalin-2-yl)-1H-pyrazol-1-yl)-N-((2-(2,6-dioxopiperidin-3-yl)-1,3-dioxoisoindolin-5-yl)methyl)acetamide

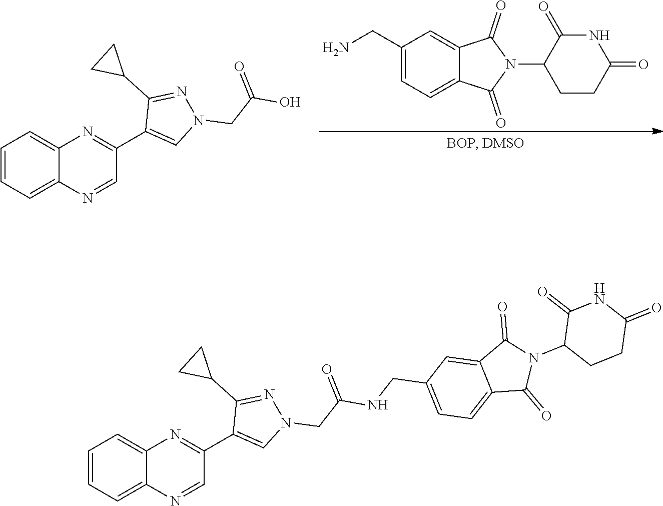 [SMILES: O=C(O)Cn1cc(-c2cnc3ccccc3n2)c(C2CC2)n1 ; NCc1ccc2c(c1)C(=O)N(C1CCC(=O)NC1=O)C2=O ; O=C(Cn1cc(-c2cnc3ccccc3n2)c(C2CC2)n1)NCc1ccc2c(c1)C(=O)N(C1CCC(=O)NC1=O)C2=O]

To a solution of 2-(3-cyclopropyl-4-(quinoxalin-3-yl)-1H-pyrazol-1-yl)acetic acid (35 mg, 0.118 mmol) and 5-(aminomethyl)-2-(2,6-dioxopiperidin-3-yl)isoindoline-1,3-dione (41 mg, 0.141 mmol) in DMSO (10 mL) was added BOP (150 mg, 0.354 mmol) and DIEA (76.1 mg, 0.59 mmol). The reaction mixture was stirred at rt overnight, before it was diluted with water, and extracted with ethyl acetate (20 mL×3). The organic layers were combined, washed with brine, dried over Na₂SO₄, and concentrated. The resulting residue was purified by flash chromatography to provide the product (16 mg, 24.2% yield) as a yellow solid. MS (ESI) m/z=564.6 [M+H]⁺.

### Example 230. 5-(((trans-3-(4-(3-Chloropyridin-2-yl)-3-cyclopropyl-1H-pyrazol-1-yl)cyclobutyl)methyl)amino)-2-(2,6-dioxopiperidin-3-yl)isoindoline-1,3-dione (GS-272)

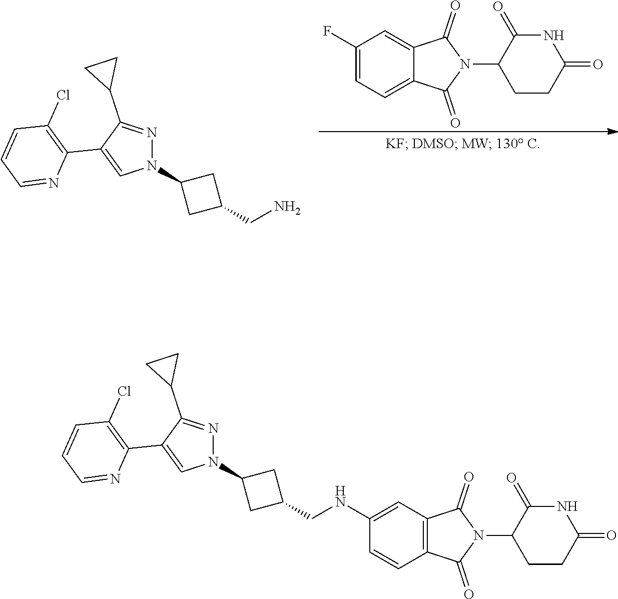 [SMILES: NC[C@H]1C[C@H](n2cc(-c3ncccc3Cl)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6ncccc6Cl)c(C6CC6)n5)C4)cc3C2=O)C(=O)N1]

GS-272 was synthesized following the similar procedure for preparing GS-178 (21.7 mg, yield: 16.1%). MS (ESI) m/z=559.5 [M+H]⁺.

### Example 231. 5-(((trans-3-(4-(6-aminopyridin-2-yl)-3-cyclopropyl-1H-pyrazol-1-yl)cyclobutyl)methyl)amino)-2-(2,6-dioxopiperidin-3-yl)isoindoline-1,3-dione (GS-273)

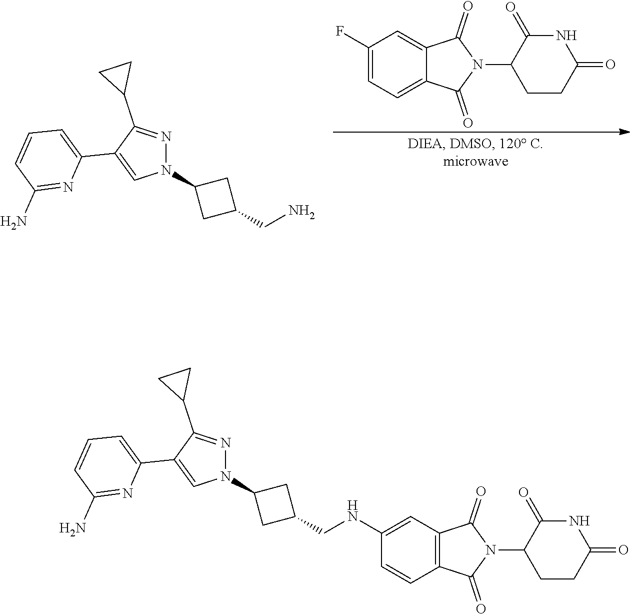 [SMILES: NC[C@H]1C[C@H](n2cc(-c3cccc(N)n3)c(C3CC3)n2)C1 ; O=C1CCC(N2C(=O)c3ccc(F)cc3C2=O)C(=O)N1 ; Nc1cccc(-c2cn([C@H]3C[C@H](CNc4ccc5c(c4)C(=O)N(C4CCC(=O)NC4=O)C5=O)C3)nc2C2CC2)n1]

GS-273 was synthesized following the similar procedure for preparing GS-178 (0.9 mg, yield: 1.4%). MS (ESI) m/z=540.7 [M+H]⁺.

### Example 232. 5-(((trans-3-(4-(6-(Azetidin-3-ylamino)pyridin-2-yl)-3-cyclopropyl-1H-pyrazol-1-yl)cyclobutyl)methyl)amino)-2-(2,6-dioxopiperidin-3-yl)isoindoline-1,3-dione (GS-274)

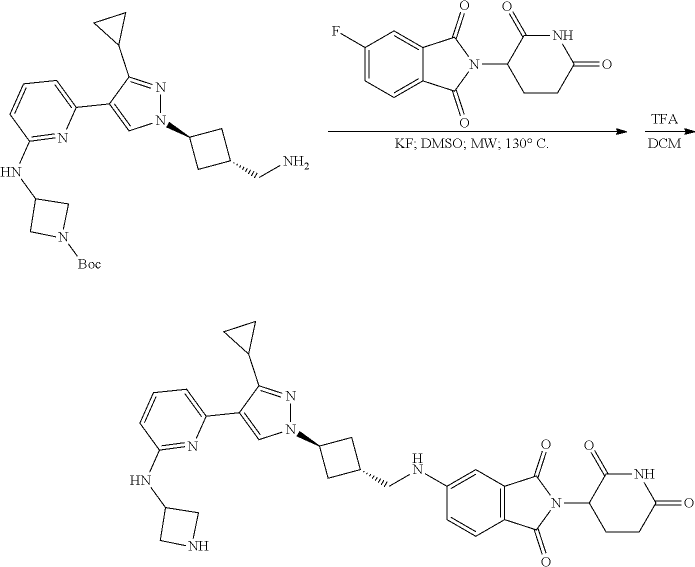 [SMILES: CC(C)(C)OC(=O)N1CC(Nc2cccc(-c3cn([C@H]4C[C@H](CN)C4)nc3C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cccc(NC7CNC7)n6)c(C6CC6)n5)C4)cc3C2=O)C(=O)N1]

GS-274 was synthesized following the similar procedure for preparing GS-174 (6 mg, yield: 13.4%). MS (ESI) m/z=595.7 [M+H]⁺.

### Example 233. 5-(((trans-3-(3-Cyclopropyl-4-(5-methoxypyridin-2-yl)-1H-pyrazol-1-yl)cyclobutyl)methyl)amino)-2-(2,6-dioxopiperidin-3-yl)isoindoline-1,3-dione (GS-275)

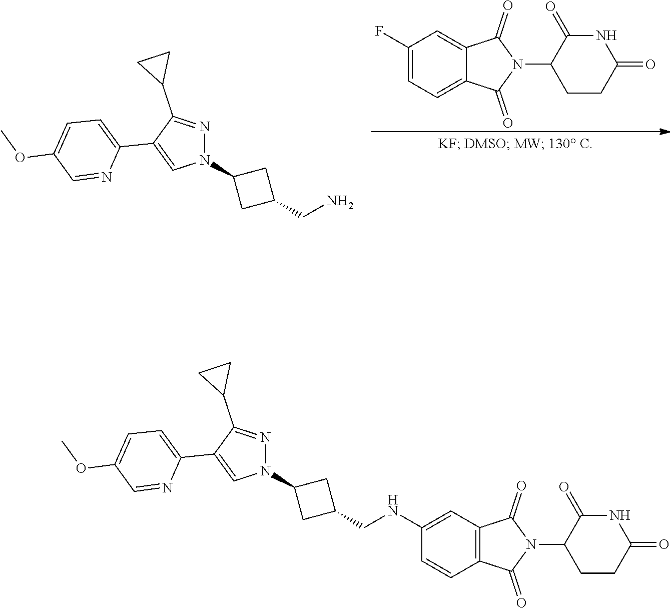 [SMILES: COc1ccc(-c2cn([C@H]3C[C@H](CN)C3)nc2C2CC2)nc1 ; O=C1CCC(N2C(=O)c3ccc(F)cc3C2=O)C(=O)N1 ; COc1ccc(-c2cn([C@H]3C[C@H](CNc4ccc5c(c4)C(=O)N(C4CCC(=O)NC4=O)C5=O)C3)nc2C2CC2)nc1]

GS-275 was synthesized following the similar procedure for preparing GS-178 (26.4 mg, yield: 21.6%). MS (ESI) m/z=555.9 [M+H]⁺.

### Example 234. 5-(((trans-3-(3-Cyclopropyl-4-(5-(dimethylamino)pyridin-2-yl)-1H-pyrazol-1-yl)cyclobutyl)methyl)amino)-2-(2,6-dioxopiperidin-3-yl)isoindoline-1,3-dione (GS-276)

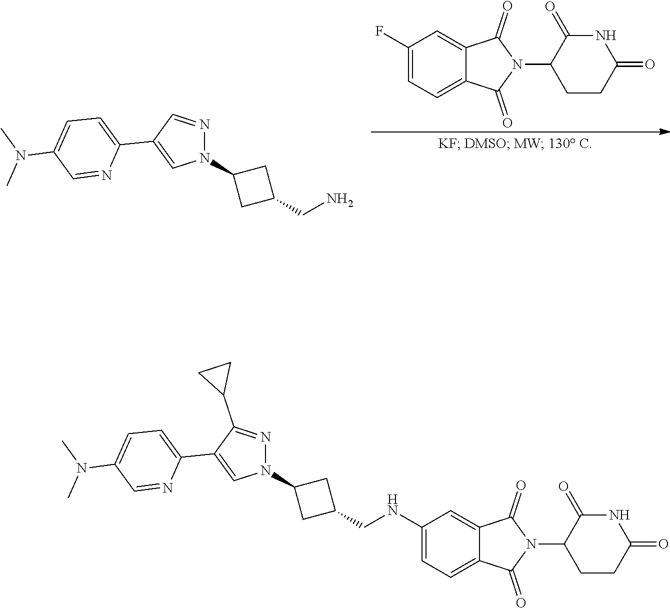 [SMILES: CN(C)c1ccc(-c2cnn([C@H]3C[C@H](CN)C3)c2)nc1 ; O=C1CCC(N2C(=O)c3ccc(F)cc3C2=O)C(=O)N1 ; CN(C)c1ccc(-c2cn([C@H]3C[C@H](CNc4ccc5c(c4)C(=O)N(C4CCC(=O)NC4=O)C5=O)C3)nc2C2CC2)nc1]

GS-276 was synthesized following the similar procedure for preparing GS-178 (32 mg, yield: 34.3%). MS (ESI) m/z=568.8 [M+H]⁺.

### Example 235. 5-(((trans-3-(3-Cyclopropyl-4-(3-(trifluoromethyl)pyridin-2-yl)-1H-pyrazol-1-yl)cyclobutyl)methyl)amino)-2-(2,6-dioxopiperidin-3-yl)isoindoline-1,3-dione (GS-277)

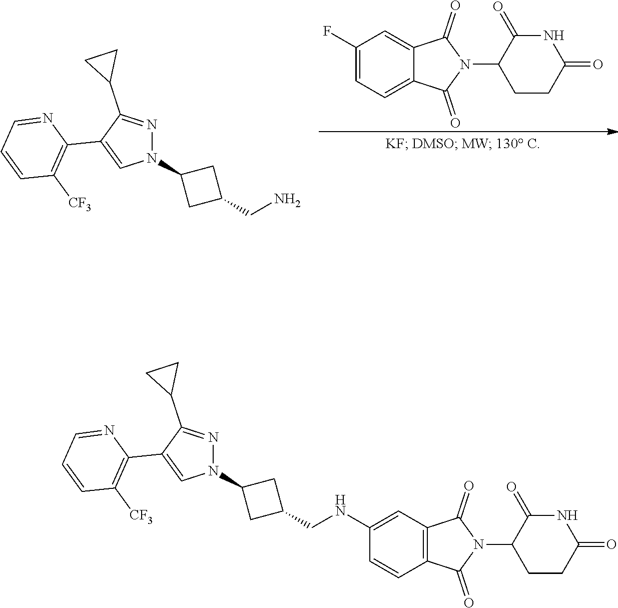 [SMILES: NC[C@H]1C[C@H](n2cc(-c3ncccc3C(F)(F)F)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6ncccc6C(F)(F)F)c(C6CC6)n5)C4)cc3C2=O)C(=O)N1]

GS-277 was synthesized following the similar procedure for preparing GS-178 (38 mg, yield: 33.4%). MS (ESI) m/z=593.7 [M+H]⁺.

### Example 236. 5-(((trans-3-(3-Cyclopropyl-4-(1H-pyrrolo[3,2-c]pyridin-4-yl)-1H-pyrazol-1-yl)cyclobutyl)methyl)amino)-2-(2,6-dioxopiperidin-3-yl)isoindoline-1,3-dione (GS-278)

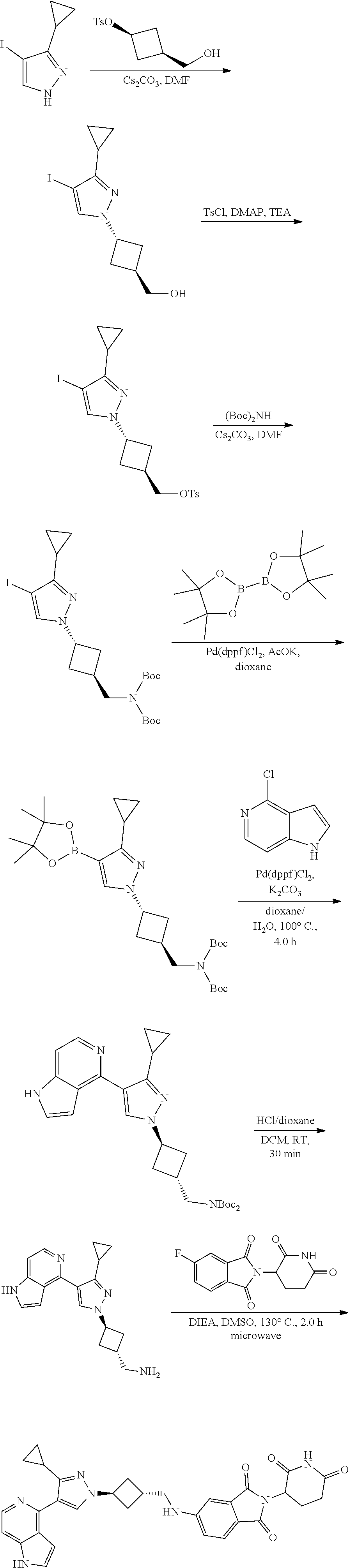

Step 1. Synthesis of (trans-3-(3-cyclopropyl-4-iodo-1H-pyrazol-1-yl)cyclobutyl)methanol

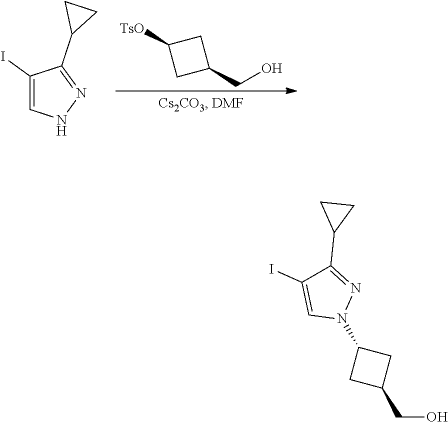

To a solution of 3-cyclopropyl-4-iodo-1H-pyrazole (24 g, 102.55 mmol) in DMF (400 mL) were added cis-3-(hydroxymethyl)cyclobutyl 4-methylbenzenesulfonate (39.43 g, 153.82 mmol) and Cs₂CO₃ (99.98 g, 307.64 mmol). After the mixture was warmed to 90° C. and stirred for 3 h, it was concentrated and purified by silica gel chromatography (petroleum ether/EtOAc=10:1 to 2:1) to provide the title compound (29 g, 88.9% yield) as a yellow oil. MS (ESI) m/z=319.4 [M+H]⁺.

Step 2. Synthesis of (trans-3-(3-cyclopropyl-4-iodo-1H-pyrazol-1-yl)cyclobutyl)methyl 4-methylbenzenesulfonate

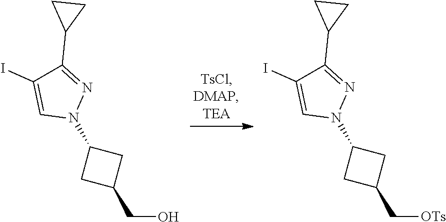 [SMILES: OC[C@H]1C[C@H](n2cc(I)c(C3CC3)n2)C1 ; Cc1ccc(S(=O)(=O)OC[C@H]2C[C@H](n3cc(I)c(C4CC4)n3)C2)cc1]

To a solution of (trans-3-(3-cyclopropyl-4-iodo-1H-pyrazol-1-yl)cyclobutyl)methanol (29 g, 91.15 mmol) in DCM (300 mL) were added TsCl (35.00 g, 182.30 mmol), DMAP (2.23 g, 18.23 mmol) and TEA (92.24 g, 911.51 mmol). After the mixture was stirred at rt for 16 h, it was concentrated and purified by silica gel chromatography (petroleum ether/EtOAc=15:1 to 10:1) to provide the title compound (31 g, 72% yield) as a colorless oil. MS (ESI) m/z=473.5 [M+H]⁺.

Step 3. Synthesis of trans-tert-butyl N-tert-butoxycarbonyl-N-[[3-(3-cyclopropyl-4-iodo-pyrazol-1-yl)cyclobutyl]methyl]carbamate

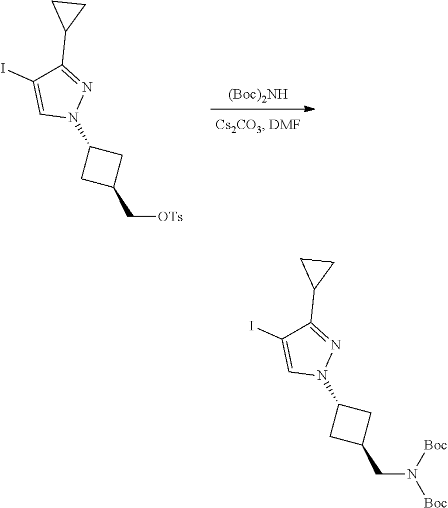 [SMILES: "(Boc)2NH" ; Cc1ccc(S(=O)(=O)OC[C@H]2C[C@H](n3cc(I)c(C4CC4)n3)C2)cc1 ; CC(C)(C)OC(=O)N(C[C@H]1C[C@H](n2cc(I)c(C3CC3)n2)C1)C(=O)OC(C)(C)C]

To a solution of (trans-3-(3-cyclopropyl-4-iodo-1H-pyrazol-1-yl)cyclobutyl)methyl 4-methylbenzenesulfonate (14 g, 29.64 mmol) and tert-butyl N-tert-butoxycarbonylcarbamate (12.88 g, 59.28 mmol) in DMF (150 mL) was added Cs₂CO₃ (28.90 g, 88.92 mmol). After the mixture was stirred at 90° C. for 4 h, it was concentrated and purified by silica gel chromatography (petroleum ether/EtOAc=15:1 to 10:1) to provide the title compound (15 g, 97.8% yield) as a yellow oil. MS (ESI) m/z=518.6 [M+H]⁺.

Step 4. Synthesis of trans-tert-butyl N-tert-butoxycarbonyl-N-[[3-[3-cyclopropyl-4-(4,4,5,5-tetramethyl-1,3,2-dioxaborolan-2-yl)pyrazol-1-yl]cyclobutyl]methyl]carbamate

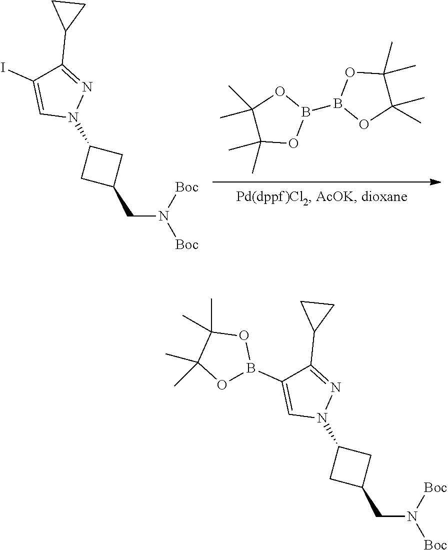

To a solution of trans-tert-butyl N-tert-butoxycarbonyl-N-[[3-(3-cyclopropyl-4-iodo-pyrazol-1-yl)cyclobutyl]methyl]carbamate (16 g, 30.92 mmol) and 4,4,5,5-tetramethyl-2-(4,4,5,5-tetramethyl-1,3,2-dioxaborolan-2-yl)-1,3,2-dioxaborolane (15.71 g, 61.85 mmol) in DMF (200 mL) were added AcOK (9.09 g, 92.77 mmol) and Pd(dppf)Cl₂ (2.26 g, 3.09 mmol) under N₂. The resulting mixture was stirred at 100° C. for 3 h, before it was cooled to rt, quenched with brine (100 mL), and extracted with EtOAc (100 mL×2). The combined organic layers were concentrated and purified by silica gel chromatography (petroleum ether/EtOAc=15:1 to 10:1) to provide the title compound (6.5 g, 40.6% yield). MS (ESI) m/z=518.8 [M+H]⁺.

Step 5. Synthesis of tert-butyl N-tert-butoxycarbonyl-N-[[trans-3-[3-cyclopropyl-4-(1H-indol-4-yl)pyrazol-1-yl]cyclobutyl]methyl]carbamate

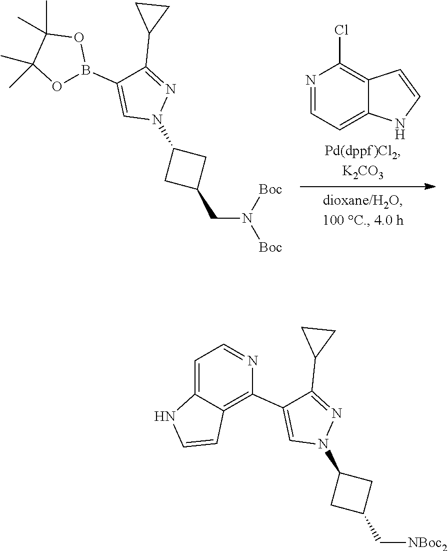

To a solution of 4-chloro-1H-pyrrolo[3,2-c]pyridine (50 mg, 0.328 mmol) and tert-butyl N-tert-butoxycarbonyl-N-[[trans3-[3-cyclopropyl-4-(4,4,5,5-tetramethyl-1,3,2-dioxaborolan-2-yl)pyrazol-1-yl]cyclobutyl]methyl]carbamate (203 mg, 0.393 mmol) in 1,4-dioxane (5.0 mL) and H₂O (0.5 mL) were added Pd(dppf)Cl₂ (24 mg, 0.032 mmol) and K₂CO₃ (136 mg, 0.983 mmol). The resulting mixture was stirred at 100° C. for 4 h, before it was concentrated and purified by reverse phase chromatography to provide the title compound (50 mg, 30.1% yield) as a light-yellow solid. MS (ESI) m/z=508.8 [M+H]⁺.

Step 6. Synthesis of (trans-3-(3-cyclopropyl-4-(1H-pyrrolo[3,2-c]pyridin-4-yl)-1H-pyrazol-1-yl)cyclobutyl)methanamine

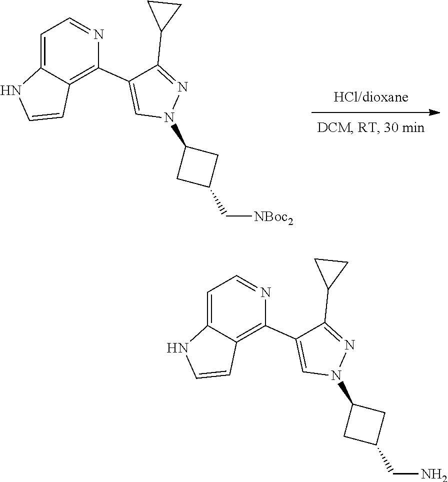

To a solution of tert-butyl N-tert-butoxycarbonyl-N-[[trans-3-[3-cyclopropyl-4-(1H-indol-4-yl)pyrazol-1-yl]cyclobutyl]methyl]carbamate (50 mg, 0.098 mmol) in DCM (1.0 mL) was added HCl/dioxane (1.0 mL, 4 M). The reaction was stirred at rt for 30 min, before it was concentrated to provide the crude product (28 mg, 92.7% yield) as light-yellow oil which was used directly in the next step.

Step 7. Synthesis of 5-(((trans-3-(3-cyclopropyl-4-(1H-pyrrolo[3,2-c]pyridin-4-yl)-1H-pyrazol-1-yl)cyclobutyl)methyl)amino)-2-(2,6-dioxopiperidin-3-yl)isoindoline-1,3-dione

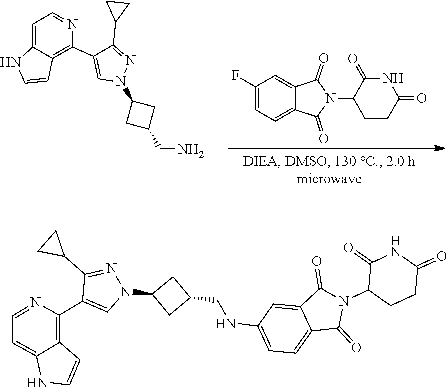 [SMILES: NC[C@H]1C[C@H](n2cc(-c3nccc4[nH]ccc34)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6nccc7[nH]ccc67)c(C6CC6)n5)C4)cc3C2=O)C(=O)N1]

To a solution of (trans-3-(3-cyclopropyl-4-(1H-pyrrolo[3,2-c]pyridin-4-yl)-1H-pyrazol-1-yl)cyclobutyl)methanamine (28 mg, 0.091 mmol) and 2-(2,6-dioxopiperidin-3-yl)-5-fluoroisoindoline-1,3-dione (56.6 mg, 0.109 mmol) in DMSO (0.5 mL) was added DIEA (0.16 mL, 0.91 mmol). The reaction was irradiated at 130° C. for 2 h in microwave, before it was concentrated and purified by reverse phase chromatography to provide the title compound (26.6 mg, 27.9% yield) as a yellow solid. MS (ESI) m/z=[M+H]⁺.

### Example 237. 5-(((trans-3-(3-Cyclopropyl-4-(1H-pyrrolo[2,3-c]pyridin-5-yl)-1H-pyrazol-1-yl)cyclobutyl)methyl)amino)-2-(2,6-dioxopiperidin-3-yl)isoindoline-1,3-dione (GS-279)

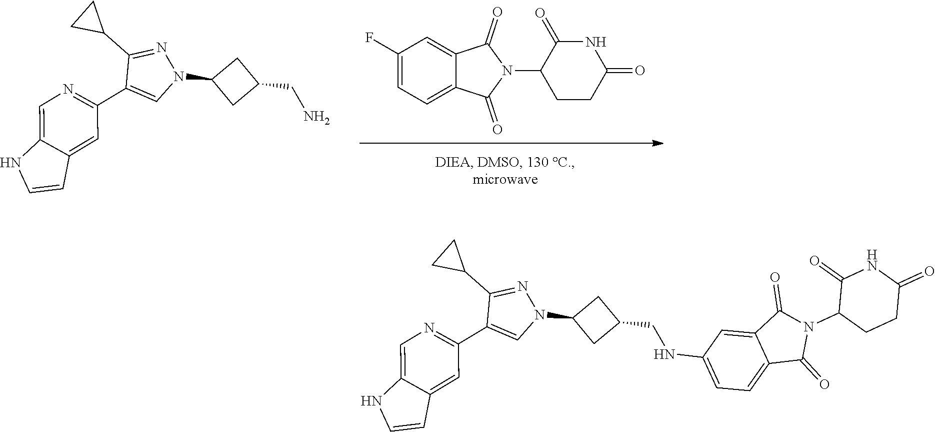 [SMILES: NC[C@H]1C[C@H](n2cc(-c3cc4cc[nH]c4cn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cc7cc[nH]c7cn6)c(C6CC6)n5)C4)cc3C2=O)C(=O)N1]

GS-279 was synthesized following the similar procedure for preparing GS-278 (4.3 mg, yield: 23.8%). MS (ESI) m/z=564.6 [M+H]⁺.

### Example 238. 5-(((trans-3-(3-Cyclopropyl-4-(2,6-naphthyridin-1-yl)-1H-pyrazol-1-yl)cyclobutyl)methyl)amino)-2-(2,6-dioxopiperidin-3-yl)isoindoline-1,3-dione (GS-280)

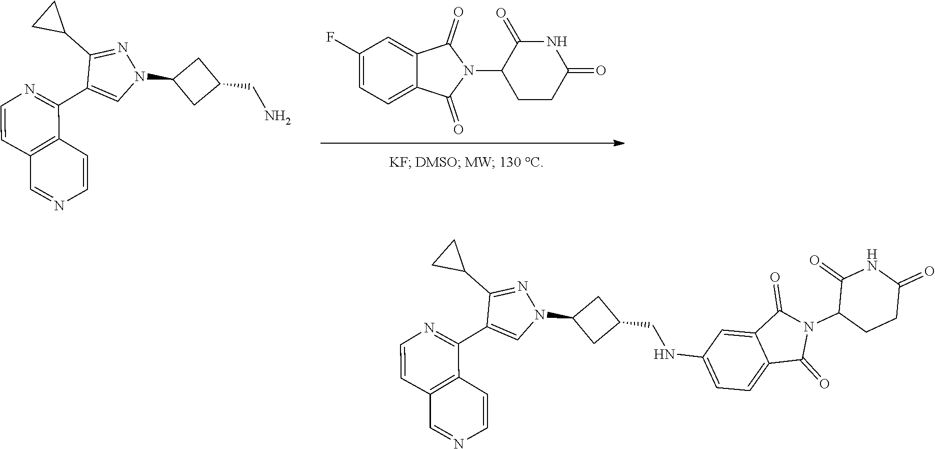 [SMILES: NC[C@H]1C[C@H](n2cc(-c3nccc4cnccc34)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6nccc7cnccc67)c(C6CC6)n5)C4)cc3C2=O)C(=O)N1]

GS-280 was synthesized following the similar procedure for preparing GS-278 (7.8 mg, yield: 35.5%). MS (ESI) m/z=576.7 [M+H]⁺.

### Example 239. 5-(((trans-3-(3-Cyclopropyl-4-(1,6-naphthyridin-5-yl)-1H-pyrazol-1-yl)cyclobutyl)methyl)amino)-2-(2,6-dioxopiperidin-3-yl)isoindoline-1,3-dione (GS-281)

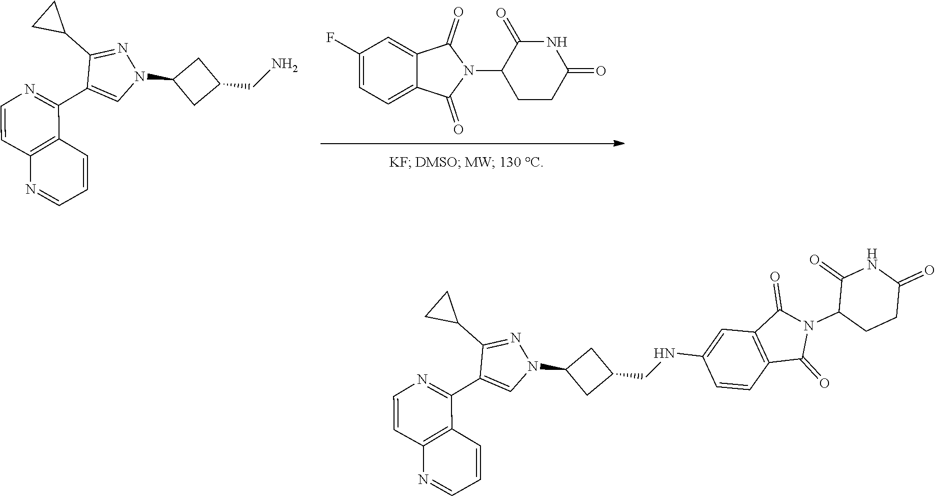 [SMILES: NC[C@H]1C[C@H](n2cc(-c3nccc4ncccc34)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6nccc7ncccc67)c(C6CC6)n5)C4)cc3C2=O)C(=O)N1]

GS-281 was synthesized following the similar procedure for preparing GS-278 (15 mg, yield: 23.8%). MS (ESI) m/z=576.7 [M+H]⁺.

### Example 240. 5-(((trans-3-(3-Cyclopropyl-4-(1H-pyrrolo[3,2-c]pyridin-6-yl)-1H-pyrazol-1-yl)cyclobutyl)methyl)amino)-2-(2,6-dioxopiperidin-3-yl)isoindoline-1,3-dione (GS-282)

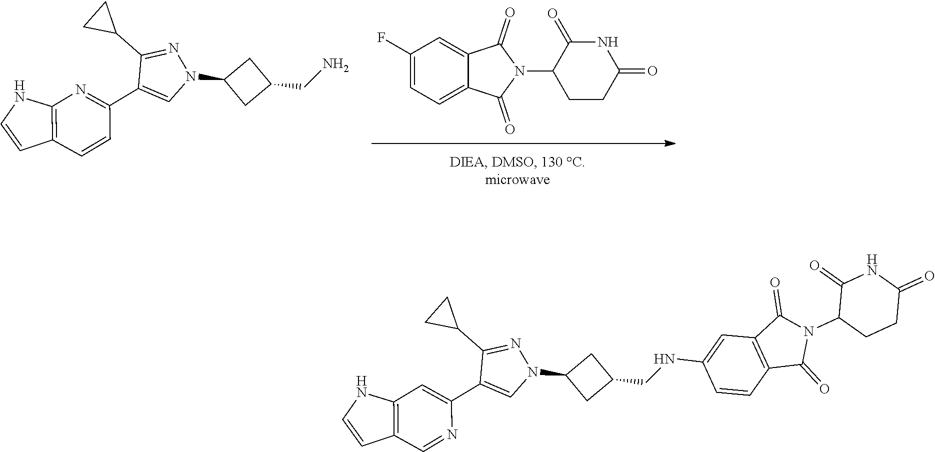 [SMILES: NC[C@H]1C[C@H](n2cc(-c3ccc4cc[nH]c4n3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cc7[nH]ccc7cn6)c(C6CC6)n5)C4)cc3C2=O)C(=O)N1]

GS-282 was synthesized following the similar procedure for preparing GS-278 (1.6 mg, yield: 3.4%). MS (ESI) m/z=564.7 [M+H]⁺.

### Example 241. 5-(((trans-3-(3-Cyclopropyl-4-(5-fluoropyridin-2-yl)-1H-pyrazol-1-yl)cyclobutyl)methyl)amino)-2-(2,6-dioxopiperidin-3-yl)isoindoline-1,3-dione (GS-283)

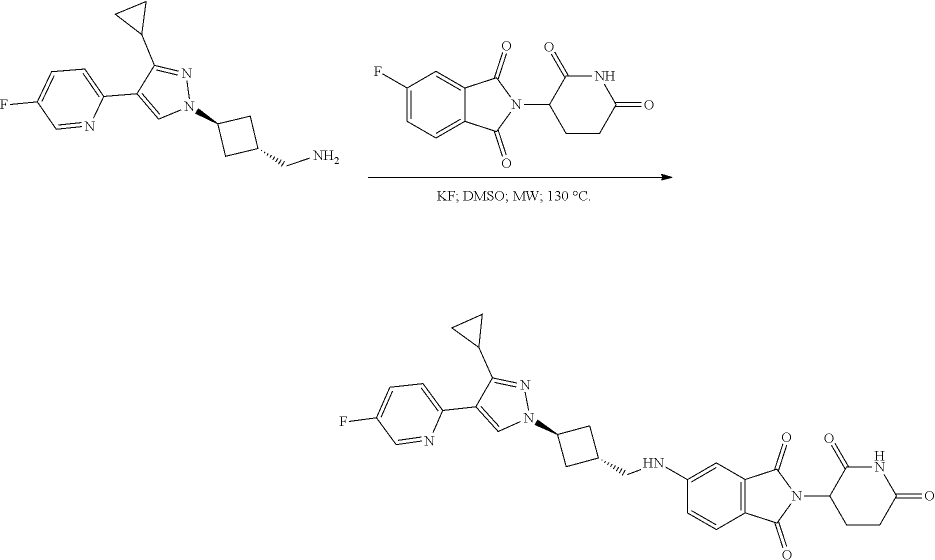 [SMILES: NC[C@H]1C[C@H](n2cc(-c3ccc(F)cn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6ccc(F)cn6)c(C6CC6)n5)C4)cc3C2=O)C(=O)N1]

GS-283 was synthesized following the similar procedure for preparing GS-178 (2.3 mg, yield: 3.2%). MS (ESI) m/z=543.6 [M+H]⁺.

### Example 242. 5-(((trans-3-(3-Cyclopropyl-4-(2,7-naphthyridin-1-yl)-1H-pyrazol-1-yl)cyclobutyl)methyl)amino)-2-(2,6-dioxopiperidin-3-yl)isoindoline-1,3-dione (GS-284)

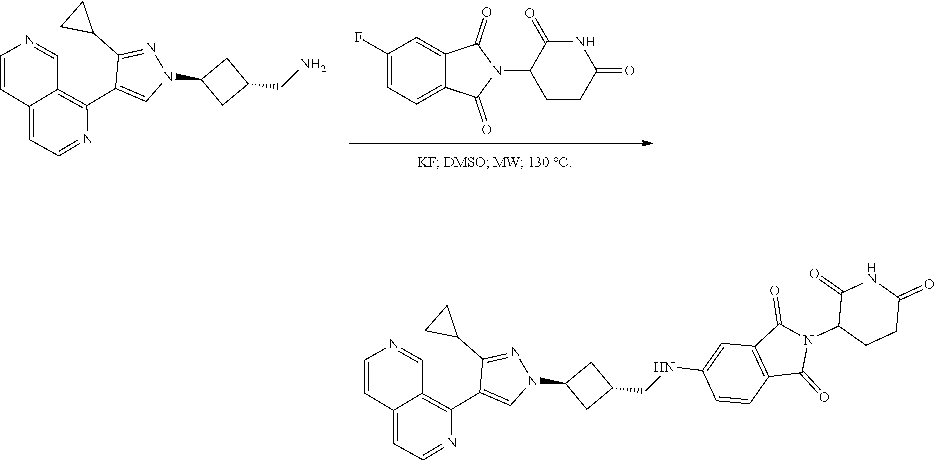 [SMILES: NC[C@H]1C[C@H](n2cc(-c3nccc4ccncc34)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6nccc7ccncc67)c(C6CC6)n5)C4)cc3C2=O)C(=O)N1]

GS-284 was synthesized following the similar procedure for preparing GS-278 (8 mg, yield: 6.2?%). MS (ESI) m/z=576.7 [M+H]⁺.

### Example 243. 5-(((trans-3-(3-Cyclopropyl-4-(pyrido[3,4-b]pyrazin-7-yl)-1H-pyrazol-1-yl)cyclobutyl)methyl)amino)-2-(2,6-dioxopiperidin-3-yl)isoindoline-1,3-dione (GS-285)

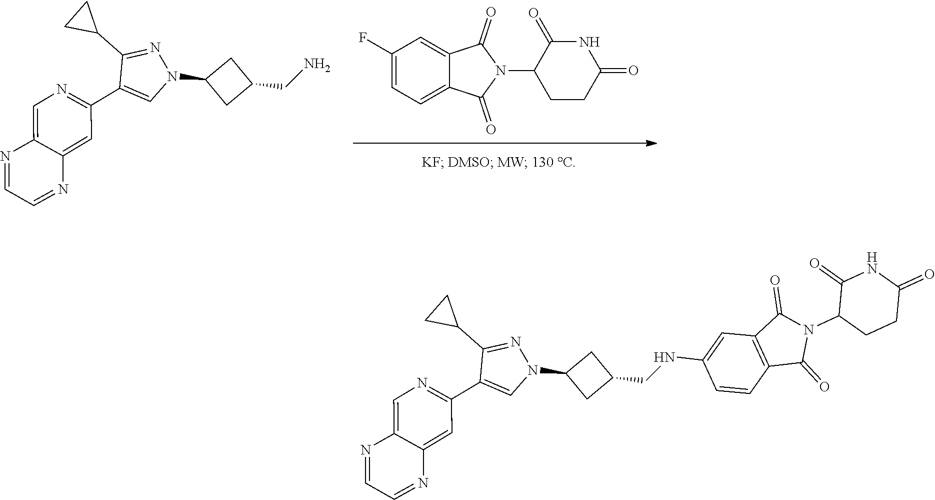 [SMILES: NC[C@H]1C[C@H](n2cc(-c3cc4nccnc4cn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cc7nccnc7cn6)c(C6CC6)n5)C4)cc3C2=O)C(=O)N1]

GS-285 was synthesized following the similar procedure for preparing GS-278 (5 mg, yield: 3.9%). MS (ESI) m/z=577.7 [M+H]⁺.

### Example 244. 5-(((trans-3-(3-Cyclopropyl-4-(1,7-naphthyridin-8-yl)-1H-pyrazol-1-yl)cyclobutyl)methyl)amino)-2-(2,6-dioxopiperidin-3-yl)isoindoline-1,3-dione (GS-286)

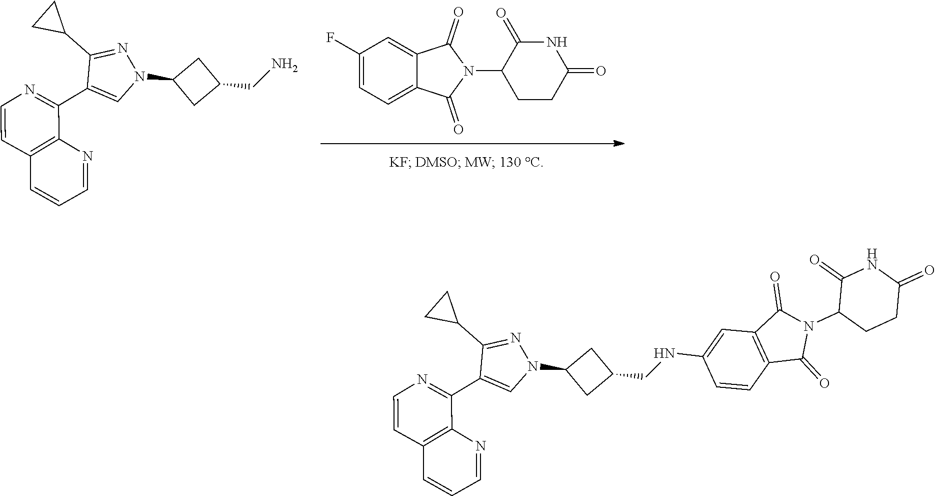 [SMILES: NC[C@H]1C[C@H](n2cc(-c3nccc4cccnc34)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6nccc7cccnc67)c(C6CC6)n5)C4)cc3C2=O)C(=O)N1]

GS-286 was synthesized following the similar procedure for preparing GS-278 (15 mg, yield: 11.6%). MS (ESI) m/z=576.7 [M+H]⁺.

### Example 245. 5-(((trans-3-(3-Cyclopropyl-4-(3-(trifluoromethoxy)pyridin-2-yl)-1H-pyrazol-1-yl)cyclobutyl)methyl)amino)-2-(2,6-dioxopiperidin-3-yl)isoindoline-1,3-dione (GS-287)

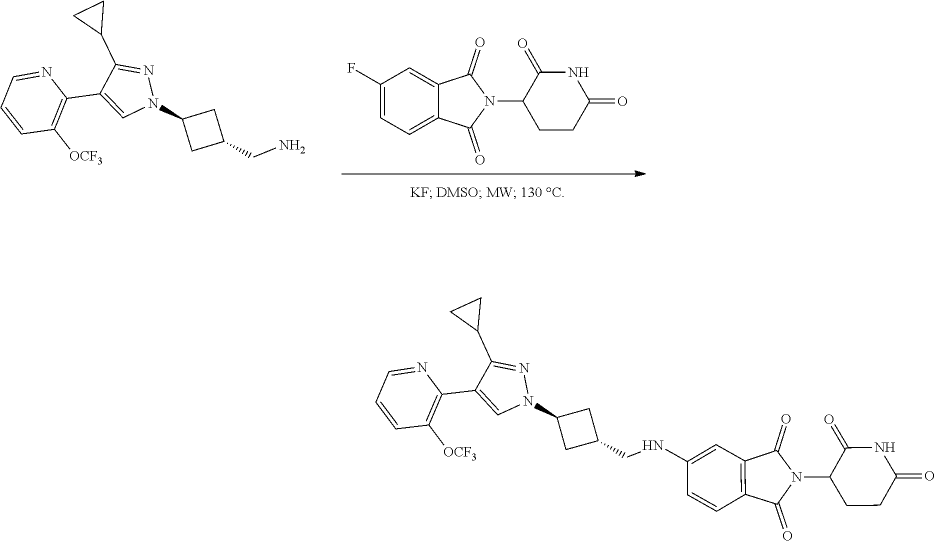 [SMILES: NC[C@H]1C[C@H](n2cc(-c3ncccc3OC(F)(F)F)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6ncccc6OC(F)(F)F)c(C6CC6)n5)C4)cc3C2=O)C(=O)N1]

GS-287 was synthesized following the similar procedure for preparing GS-278 (36 mg, yield: 15.4%). MS (ESI) m/z=609.7 [M+H]⁺.

### Example 246. 5-(((trans-3-(3-Cyclopropyl-4-(1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-288)

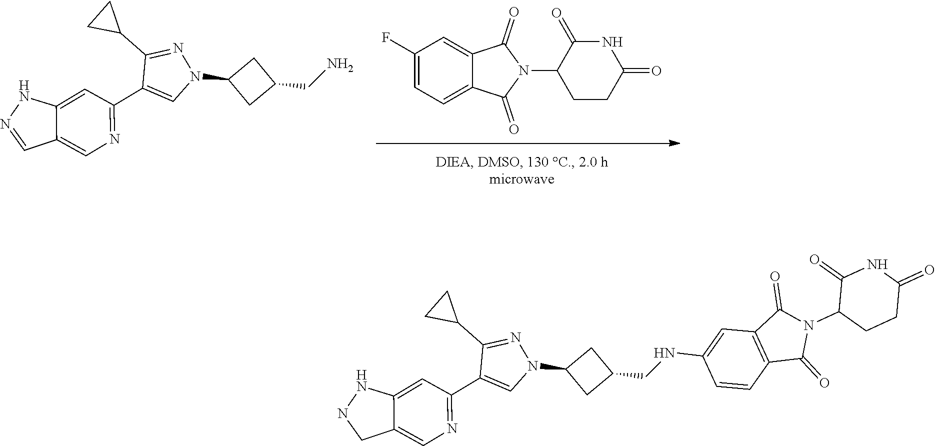 [SMILES: NC[C@H]1C[C@H](n2cc(-c3cc4[nH]ncc4cn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cc7c(cn6)CNN7)c(C6CC6)n5)C4)cc3C2=O)C(=O)N1]

GS-288 was synthesized following the similar procedure for preparing GS-278 (3 mg, yield: 14.8%). MS (ESI) m/z=565.6 [M+H]⁺.

### Example 247. 5-(((trans-3-(3-Cyclopropyl-4-(2-fluorophenyl)-1H-pyrazol-1-yl)cyclobutyl)methyl)amino)-2-(2,6-dioxopiperidin-3-yl)isoindoline-1,3-dione (GS-289)

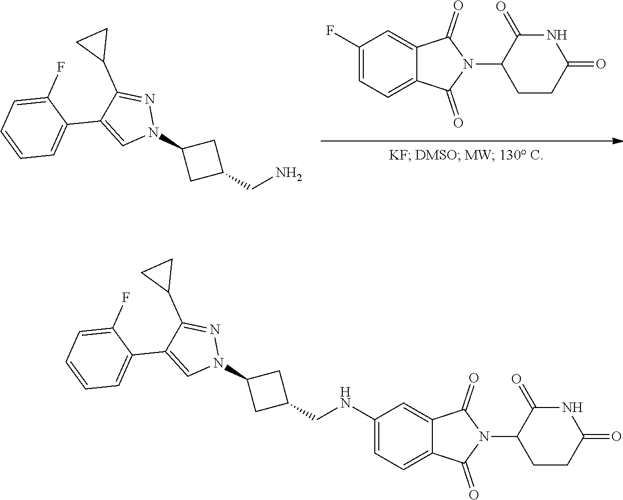 [SMILES: NC[C@H]1C[C@H](n2cc(-c3ccccc3F)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6ccccc6F)c(C6CC6)n5)C4)cc3C2=O)C(=O)N1]

GS-289 was synthesized following the similar procedure for preparing GS-278 (3 mg, yield: 14.8%) MS (ESI) m/z=542.7 [M+H]⁺.

### Example 248. 5-(((trans-3-(3-Cyclopropyl-4-(4-fluoropyridin-2-yl)-1H-pyrazol-1-yl)cyclobutyl)methyl)amino)-2-(2,6-dioxopiperidin-3-yl)isoindoline-1,3-dione (GS-290)

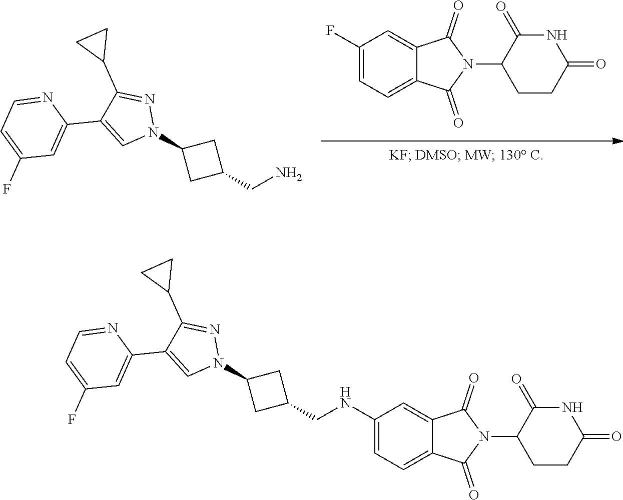 [SMILES: NC[C@H]1C[C@H](n2cc(-c3cc(F)ccn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cc(F)ccn6)c(C6CC6)n5)C4)cc3C2=O)C(=O)N1]

GS-290 was synthesized following the similar procedure for preparing GS-278 (6 mg, yield: 19.6%). MS (ESI) m/z=543.6 [M+H]⁺.

### Example 249. 5-(((trans-3-(3-Cyclopropyl-4-(1H-pyrrolo[2,3-c]pyridin-7-yl)-1H-pyrazol-1-yl)cyclobutyl)methyl)amino)-2-(2,6-dioxopiperidin-3-yl)isoindoline-1,3-dione (GS-291)

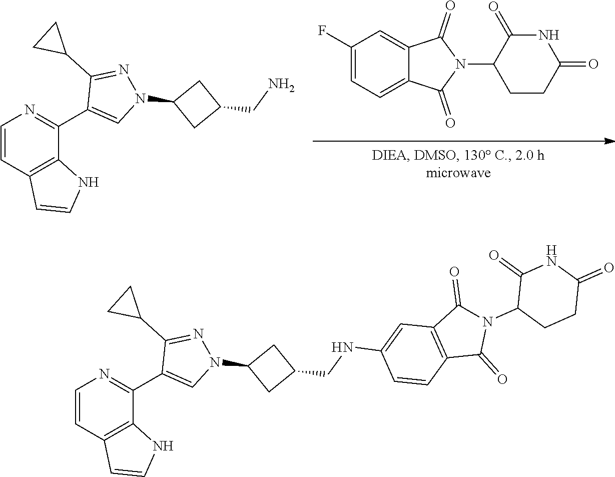 [SMILES: NC[C@H]1C[C@H](n2cc(-c3nccc4cc[nH]c34)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6nccc7cc[nH]c67)c(C6CC6)n5)C4)cc3C2=O)C(=O)N1]

GS-291 was synthesized following the similar procedure for preparing GS-278 (1.4 mg, yield: 5.9%). MS (ESI) m/z=564.8 [M+H]⁺.

### Example 250. 5-(((trans-3-(3-Cyclopropyl-4-(6-((1-methylazetidin-3-yl)amino)pyridin-2-yl)-1H-pyrazol-1-yl)cyclobutyl)methyl)amino)-2-(2,6-dioxopiperidin-3-yl)isoindoline-1,3-dione (GS-292)

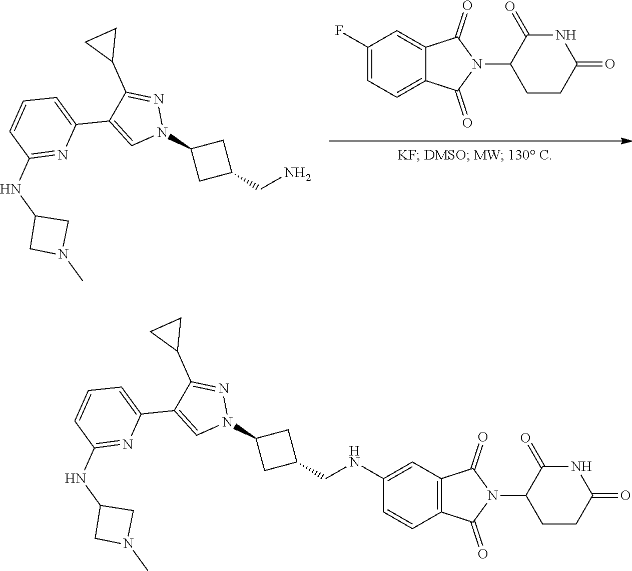 [SMILES: CN1CC(Nc2cccc(-c3cn([C@H]4C[C@H](CN)C4)nc3C3CC3)n2)C1 ; O=C1CCC(N2C(=O)c3ccc(F)cc3C2=O)C(=O)N1 ; CN1CC(Nc2cccc(-c3cn([C@H]4C[C@H](CNc5ccc6c(c5)C(=O)N(C5CCC(=O)NC5=O)C6=O)C4)nc3C3CC3)n2)C1]

GS-292 was synthesized following the similar procedure for preparing GS-178 (1.1 mg, yield: 2.3%). MS (ESI) m/z=609.7 [M+H]⁺.

### Example 251. 5-(((trans-3-(3-Cyclopropyl-4-(1H-pyrazolo[3,4-c]pyridin-5-yl)-1H-pyrazol-1-yl)cyclobutyl)methyl)amino)-2-(2,6-dioxopiperidin-3-yl)isoindoline-1,3-dione (GS-293)

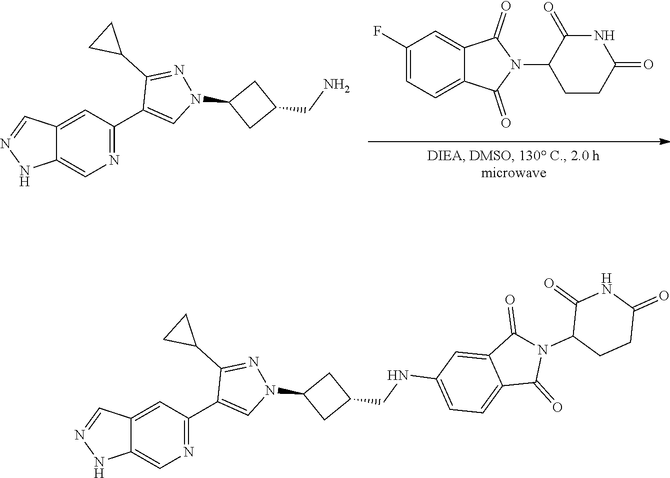 [SMILES: NC[C@H]1C[C@H](n2cc(-c3cc4cn[nH]c4cn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cc7cn[nH]c7cn6)c(C6CC6)n5)C4)cc3C2=O)C(=O)N1]

GS-293 was synthesized following the similar procedure for preparing GS-278 (1.8 mg, yield: 22.4%). MS (ESI) m/z=565.3 [M+H]⁺.

### Example 252. 5-(((trans-3-(3-Cyclopropyl-4-(4-(piperidin-4-ylamino)pyridin-2-yl)-1H-pyrazol-1-yl)cyclobutyl)methyl)amino)-2-(2,6-dioxopiperidin-3-yl)isoindoline-1,3-dione (GS-294)

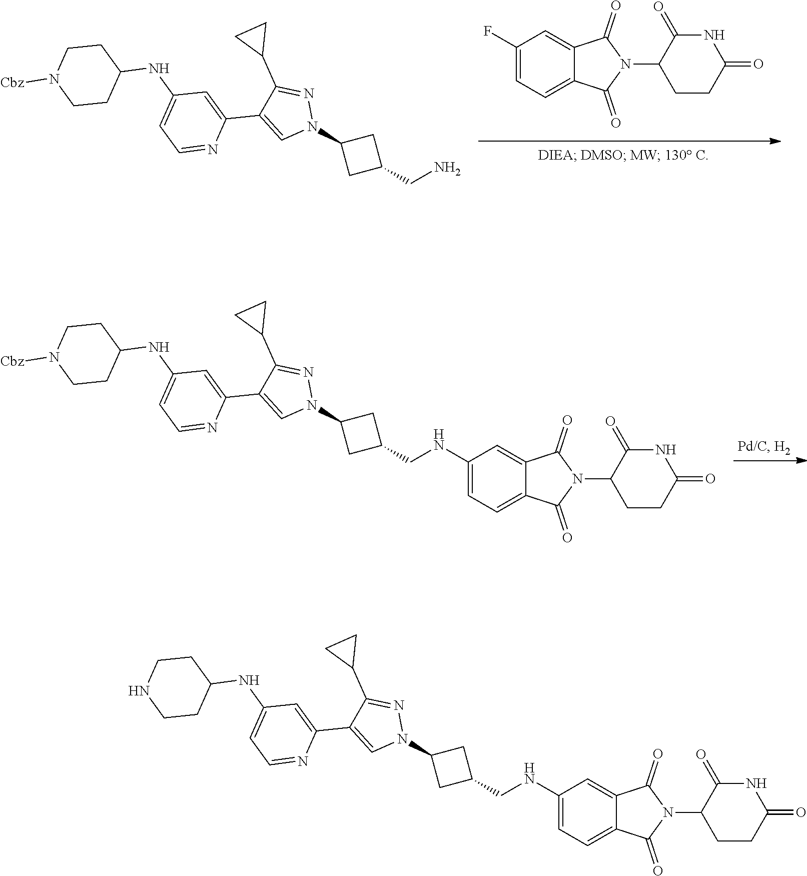 [SMILES: NC[C@H]1C[C@H](n2cc(-c3cc(NC4CCN(C(=O)OCc5ccccc5)CC4)ccn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cc(NC7CCN(C(=O)OCc8ccccc8)CC7)ccn6)c(C6CC6)n5)C4)cc3C2=O)C(=O)N1 ; O=C1CCC(N2C(=O)c3ccc(NC[C@H]4C[C@H](n5cc(-c6cc(NC7CCNCC7)ccn6)c(C6CC6)n5)C4)cc3C2=O)C(=O)N1]

Step 1. Synthesis of benzyl 4-((2-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)pyridin-4-yl)amino)piperidine-1-carboxylate

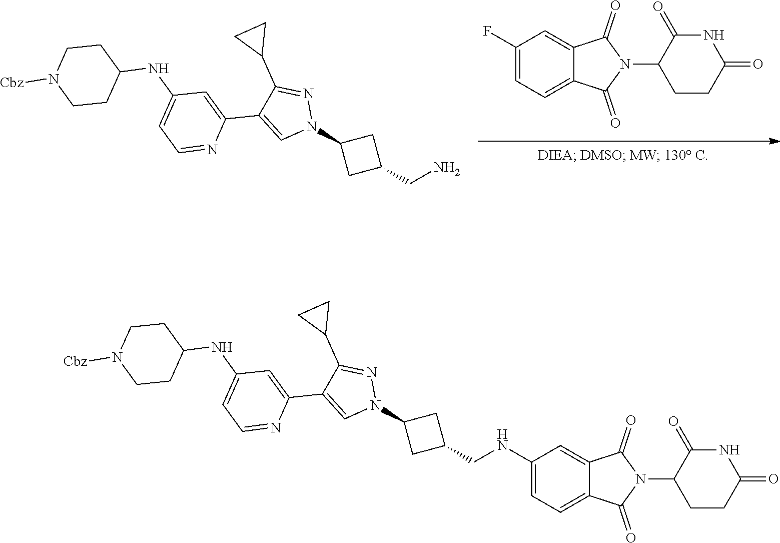 [SMILES: NC[C@H]1C[C@H](n2cc(-c3cc(NC4CCN(C(=O)OCc5ccccc5)CC4)ccn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cc(NC7CCN(C(=O)OCc8ccccc8)CC7)ccn6)c(C6CC6)n5)C4)cc3C2=O)C(=O)N1]

The mixture of benzyl 4-((2-(1-(trans-3-(aminomethyl)cyclobutyl)-3-cyclopropyl-1H-pyrazol-4-yl)pyridin-4-yl)amino)piperidine-1-carboxylate (26.5 mg, 53 μmol), 2-(2,6-dioxo-3-piperidyl)-5-fluoro-isoindoline-1,3-dione (29 mg, 106 μmol) and DIEA (137 mg, 1.06 mmol) in DMSO (1.5 mL) was irradiated at 130° C. for 2 h in microwave. The mixture was purified by reverse phase column chromatography to provide the title compound (16 mg, 39.9% yield) as a yellow solid. MS (ESI) m/z=757.8 [M+H]⁺.

Step 2. Synthesis of 5-(((trans-3-(3-cyclopropyl-4-(4-(piperidin-4-ylamino)pyridin-2-yl)-1H-pyrazol-1-yl)cyclobutyl)methyl)amino)-2-(2,6-dioxopiperidin-3-yl)isoindoline-1,3-dione

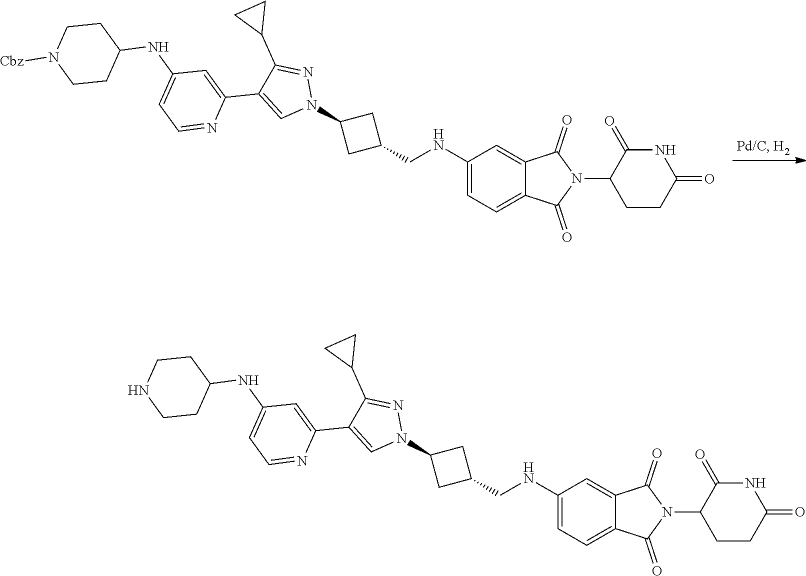 [SMILES: O=C1CCC(N2C(=O)c3ccc(NC[C@H]4C[C@H](n5cc(-c6cc(NC7CCN(C(=O)OCc8ccccc8)CC7)ccn6)c(C6CC6)n5)C4)cc3C2=O)C(=O)N1 ; O=C1CCC(N2C(=O)c3ccc(NC[C@H]4C[C@H](n5cc(-c6cc(NC7CCNCC7)ccn6)c(C6CC6)n5)C4)cc3C2=O)C(=O)N1]

To a solution of benzyl 4-((2-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)pyridin-4-yl)amino)piperidine-1-carboxylate (16 mg, 21 μmol) in EtOH (5 mL) was added Pd/C (20 mg, 5%). The reaction mixture is stirred under an hydrogen atmosphere (45 psi) at rt for 12 h. The reaction mixture was filtered, and the filtrate was concentrated. The resulting residue was purified by reverse phase column chromatography to provide the title compound (2.6 mg, 19.8% yield) as a yellow solid. MS (ESI) m/z=623.53 [M+H]⁺.

### Example 253. 5-(((trans-3-(3-Cyclopropyl-4-(4-((1-methylpiperidin-4-yl)amino)pyridin-2-yl)-1H-pyrazol-1-yl)cyclobutyl)methyl)amino)-2-(2,6-dioxopiperidin-3-yl)isoindoline-1,3-dione (GS-295)

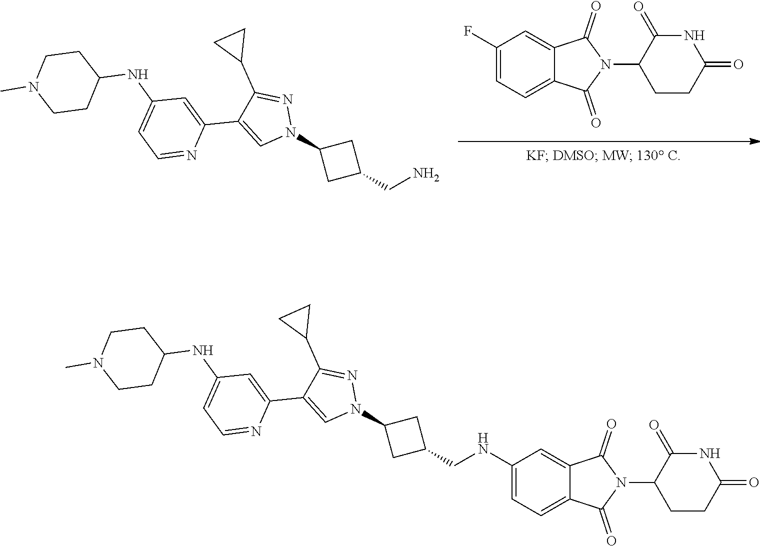 [SMILES: CN1CCC(Nc2ccnc(-c3cn([C@H]4C[C@H](CN)C4)nc3C3CC3)c2)CC1 ; O=C1CCC(N2C(=O)c3ccc(F)cc3C2=O)C(=O)N1 ; CN1CCC(Nc2ccnc(-c3cn([C@H]4C[C@H](CNc5ccc6c(c5)C(=O)N(C5CCC(=O)NC5=O)C6=O)C4)nc3C3CC3)c2)CC1]

GS-295 was synthesized following the similar procedure for preparing GS-178 (4.4 mg, yield: 34.2%). MS (ESI) m/z=637.5 [M+H]⁺.

### Example 254. 5-(((trans-3-(3-Cyclopropyl-4-(3-fluoropyridin-2-yl)-1H-pyrazol-1-yl)cyclobutyl)methyl)amino)-2-(2,6-dioxopiperidin-3-yl)isoindoline-1,3-dione (GS-296)

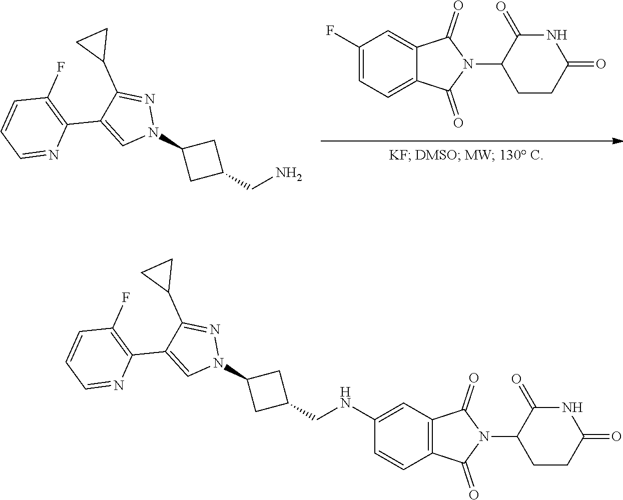 [SMILES: NC[C@H]1C[C@H](n2cc(-c3ncccc3F)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6ncccc6F)c(C6CC6)n5)C4)cc3C2=O)C(=O)N1]

GS-296 was synthesized following the similar procedure for preparing GS-178 (2.1 mg, yield: 10.5%). MS (ESI) m/z=543.7 [M+H]⁺.

### Example 255. 5-(((trans-3-(3-Cyclopropyl-4-(5-(piperazin-1-yl)pyridin-2-yl)-1H-pyrazol-1-yl)cyclobutyl)methyl)amino)-2-(2,6-dioxopiperidin-3-yl)isoindoline-1,3-dione (GS-297)

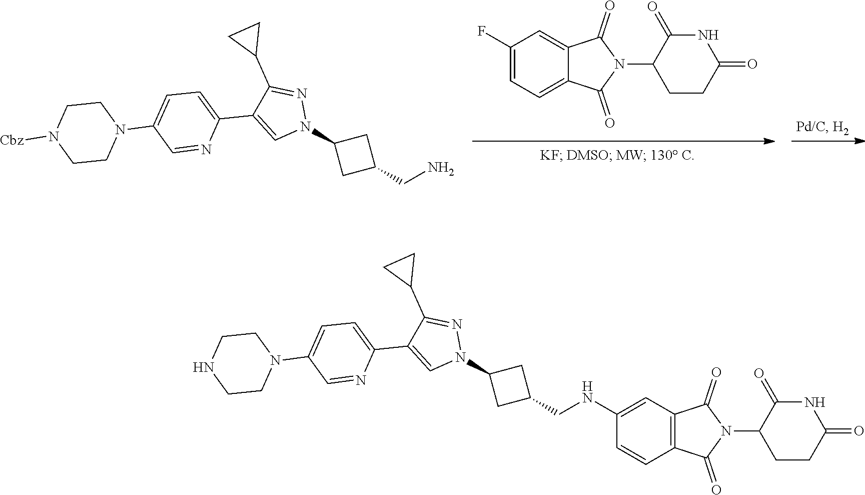 [SMILES: NC[C@H]1C[C@H](n2cc(-c3ccc(N4CCN(C(=O)OCc5ccccc5)CC4)cn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6ccc(N7CCNCC7)cn6)c(C6CC6)n5)C4)cc3C2=O)C(=O)N1]

GS-297 was synthesized following the similar procedure for preparing GS-294 (1.2 mg, yield: 3.4%). MS (ESI) m/z=609.5 [M+H]⁺.

### Example 256. 5-(((trans-3-(3-Cyclopropyl-4-(5-(4-methylpiperazin-1-yl)pyridin-2-yl)-1H-pyrazol-1-yl)cyclobutyl)methyl)amino)-2-(2,6-dioxopiperidin-3-yl)isoindoline-1,3-dione (GS-298)

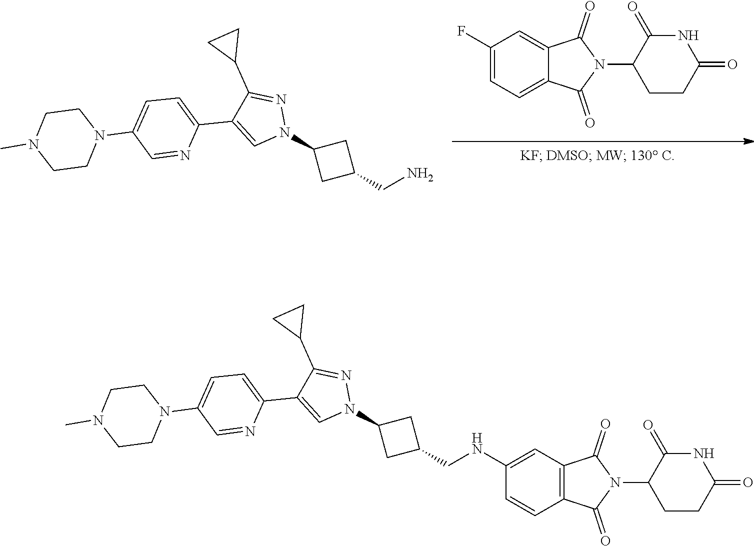 [SMILES: CN1CCN(c2ccc(-c3cn([C@H]4C[C@H](CN)C4)nc3C3CC3)nc2)CC1 ; O=C1CCC(N2C(=O)c3ccc(F)cc3C2=O)C(=O)N1 ; CN1CCN(c2ccc(-c3cn([C@H]4C[C@H](CNc5ccc6c(c5)C(=O)N(C5CCC(=O)NC5=O)C6=O)C4)nc3C3CC3)nc2)CC1]

GS-298 was synthesized following the similar procedure for preparing GS-278 (4.4 mg, yield: 23.5%). MS (ESI) m/z=623.5 [M+H]⁺.

### Example 257. 5-(((trans-3-(3-Cyclopropyl-4-(1H-pyrazolo[4,3-c]pyridin-4-yl)-1H-pyrazol-1-yl)cyclobutyl)methyl)amino)-2-(2,6-dioxopiperidin-3-yl)isoindoline-1,3-dione (GS-299)

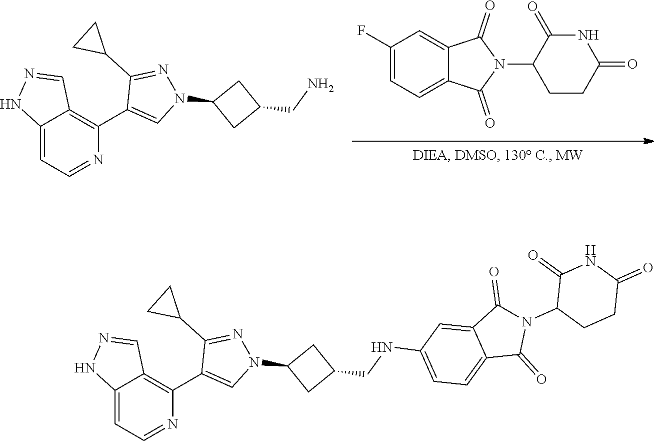 [SMILES: NC[C@H]1C[C@H](n2cc(-c3nccc4[nH]ncc34)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6nccc7[nH]ncc67)c(C6CC6)n5)C4)cc3C2=O)C(=O)N1]

GS-299 was synthesized following the similar procedure for preparing GS-278 (4.2 mg, yield: 15.3%). MS (ESI) m/z=565.4 [M+H]⁺.

### Example 258. 5-(((trans-3-(4-Cyclopropyl-3-(3-(4-methylpiperazin-1-yl)pyridin-2-yl)-1H-pyrazol-1-yl)cyclobutyl)methyl)amino)-2-(2,6-dioxopiperidin-3-yl)isoindoline-1,3-dione (GS-300)

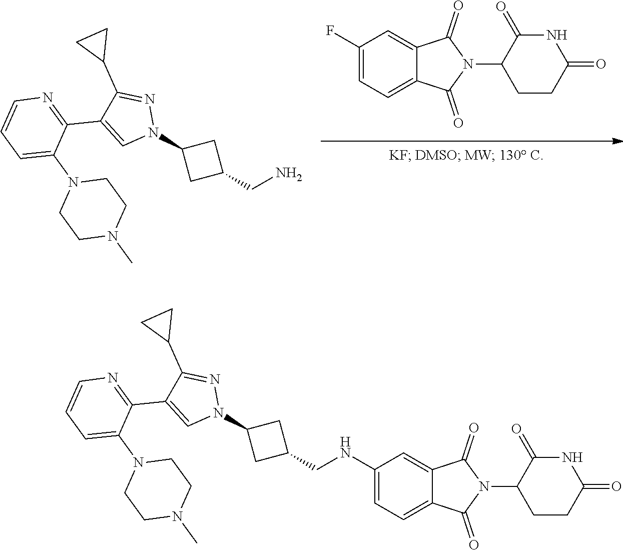 [SMILES: CN1CCN(c2cccnc2-c2cn([C@H]3C[C@H](CN)C3)nc2C2CC2)CC1 ; O=C1CCC(N2C(=O)c3ccc(F)cc3C2=O)C(=O)N1 ; CN1CCN(c2cccnc2-c2cn([C@H]3C[C@H](CNc4ccc5c(c4)C(=O)N(C4CCC(=O)NC4=O)C5=O)C3)nc2C2CC2)CC1]

GS-300 was synthesized following the similar procedure for preparing GS-178 (16 mg, yield: 18.2%). MS (ESI) m/z=623.8 [M+H]⁺.

### Example 259. 5-(((trans-3-(3-Cyclopropyl-4-(5-(methylamino)pyridin-2-yl)-1H-pyrazol-1-yl)cyclobutyl)methyl)amino)-2-(2,6-dioxopiperidin-3-yl)isoindoline-1,3-dione (GS-301)

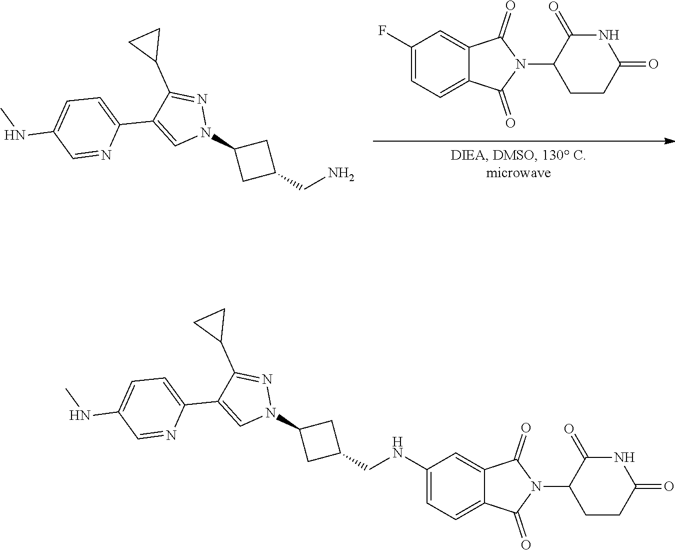 [SMILES: CNc1ccc(-c2cn([C@H]3C[C@H](CN)C3)nc2C2CC2)nc1 ; O=C1CCC(N2C(=O)c3ccc(F)cc3C2=O)C(=O)N1 ; CNc1ccc(-c2cn([C@H]3C[C@H](CNc4ccc5c(c4)C(=O)N(C4CCC(=O)NC4=O)C5=O)C3)nc2C2CC2)nc1]

GS-301 was synthesized following the similar procedure for preparing GS-278 (1.7 mg, yield: 12.3%). MS (ESI) m/z=554.7 [M+H]⁺.

### Example 260. 5-(((trans-3-(3-Cyclopropyl-4-(5-(piperidin-4-ylamino)pyridin-2-yl)-1H-pyrazol-1-yl)cyclobutyl)methyl)amino)-2-(2,6-dioxopiperidin-3-yl)isoindoline-1,3-dione (GS-302)

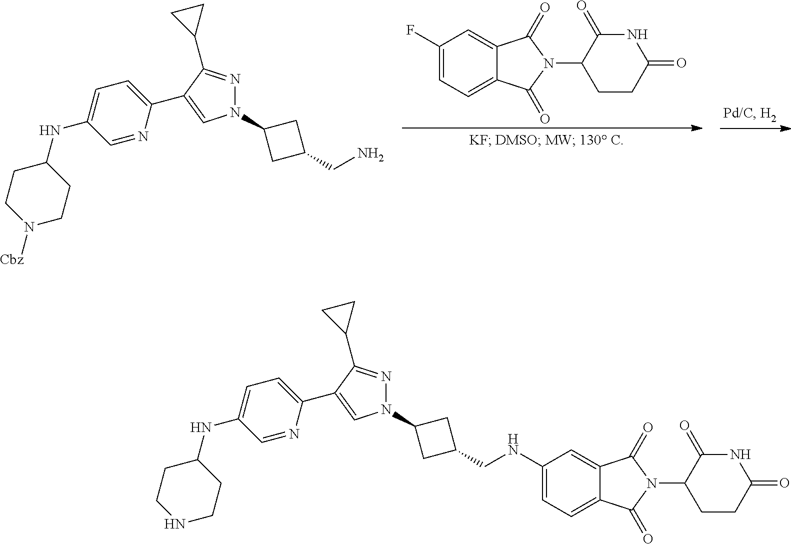 [SMILES: NC[C@H]1C[C@H](n2cc(-c3ccc(NC4CCN(C(=O)OCc5ccccc5)CC4)cn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6ccc(NC7CCNCC7)cn6)c(C6CC6)n5)C4)cc3C2=O)C(=O)N1]

GS-302 was synthesized following the similar procedure for preparing GS-294 (3.6 mg, yield: 32.2%). MS (ESI) m/z=623.8 [M+H]⁺.

### Example 261. 5-(((trans-3-(3-Cyclopropyl-4-(5-((1-methylpiperidin-4-yl)amino)pyridin-2-yl)-1H-pyrazol-1-yl)cyclobutyl)methyl)amino)-2-(2,6-dioxopiperidin-3-yl)isoindoline-1,3-dione (GS-303)

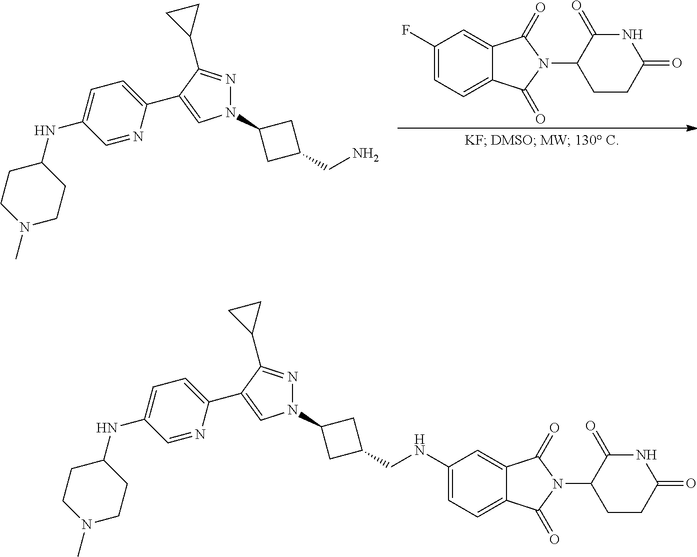 [SMILES: CN1CCC(Nc2ccc(-c3cn([C@H]4C[C@H](CN)C4)nc3C3CC3)nc2)CC1 ; O=C1CCC(N2C(=O)c3ccc(F)cc3C2=O)C(=O)N1 ; CN1CCC(Nc2ccc(-c3cn([C@H]4C[C@H](CNc5ccc6c(c5)C(=O)N(C5CCC(=O)NC5=O)C6=O)C4)nc3C3CC3)nc2)CC1]

GS-303 was synthesized following the similar procedure for preparing GS-178 (0.63 mg, yield: 7.5%). MS (ESI) m/z=637.7 [M+H]⁺.

### Example 262. 5-(((trans-3-(3-Cyclopropyl-4-(3-(piperidin-4-ylamino)pyridin-2-yl)-1H-pyrazol-1-yl)cyclobutyl)methyl)amino)-2-(2,6-dioxopiperidin-3-yl)isoindoline-1,3-dione (GS-304)

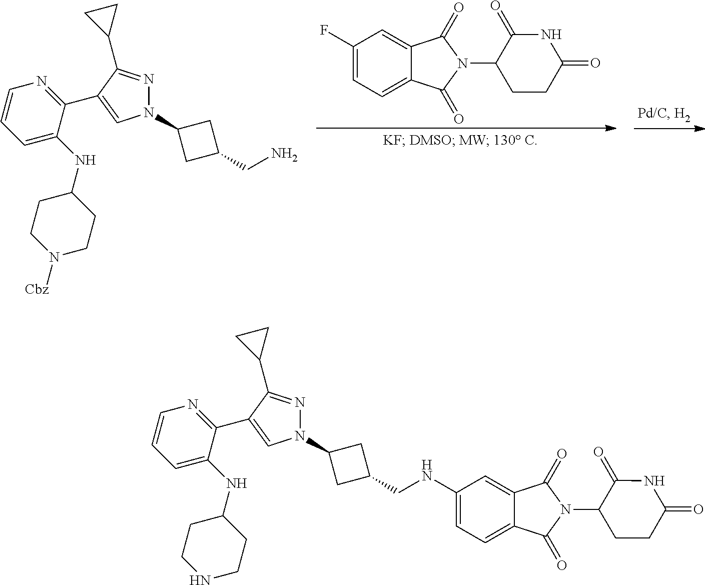 [SMILES: NC[C@H]1C[C@H](n2cc(-c3ncccc3NC3CCN(C(=O)OCc4ccccc4)CC3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6ncccc6NC6CCNCC6)c(C6CC6)n5)C4)cc3C2=O)C(=O)N1]

GS-304 was synthesized following the similar procedure for preparing GS-294 (1.1 mg, yield: 10.7%). MS (ESI) m/z=623.7 [M+H]⁺.

### Example 263. 5-(((trans-3-(3-Cyclopropyl-4-(5-fluoro-6-methylpyridin-2-yl)-1H-pyrazol-1-yl)cyclobutyl)methyl)amino)-2-(2,6-dioxopiperidin-3-yl)isoindoline-1,3-dione (GS-305)

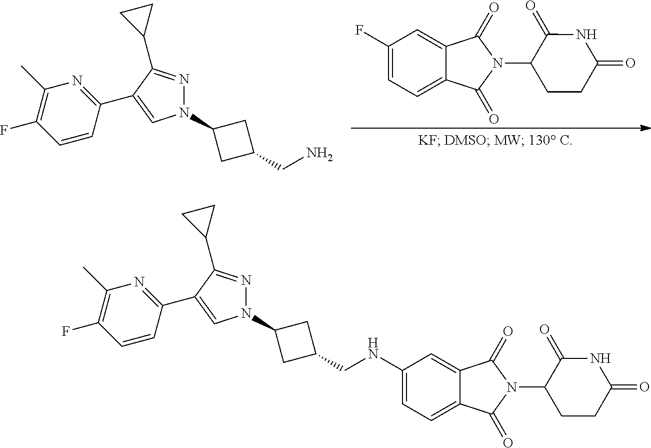

GS-305 was synthesized following the similar procedure for preparing GS-278 (3.3 mg, yield: 10.3%). MS (ESI) m/z=557.8 [M+H]⁺.

### Example 264. 5-(((trans-3-(3-Cyclopropyl-4-(4-(methylamino)pyridin-2-yl)-1H-pyrazol-1-yl)cyclobutyl)methyl)amino)-2-(2,6-dioxopiperidin-3-yl)isoindoline-1,3-dione (GS-306)

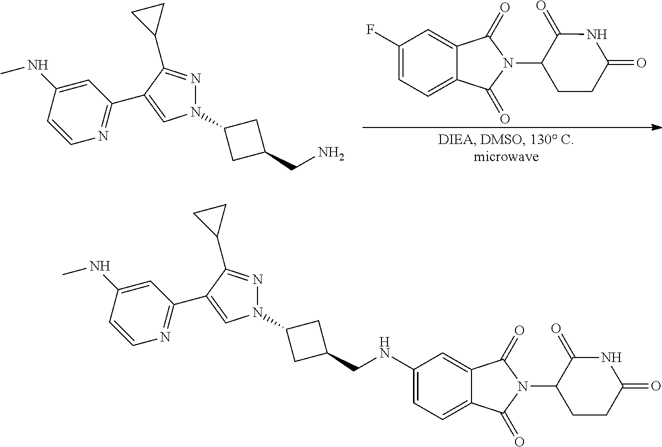

GS-306 was synthesized following the similar procedure for preparing GS-278 (3.1 mg, yield: 19.3%) MS (ESI) m/z=554.6 [M+H]⁺.

### Example 265. 5-(((trans-3-(3-Cyclopropyl-4-(1H-imidazo[4,5-c]pyridin-6-yl)-1H-pyrazol-1-yl)cyclobutyl)methyl)amino)-2-(2,6-dioxopiperidin-3-yl)isoindoline-1,3-dione (GS-307)

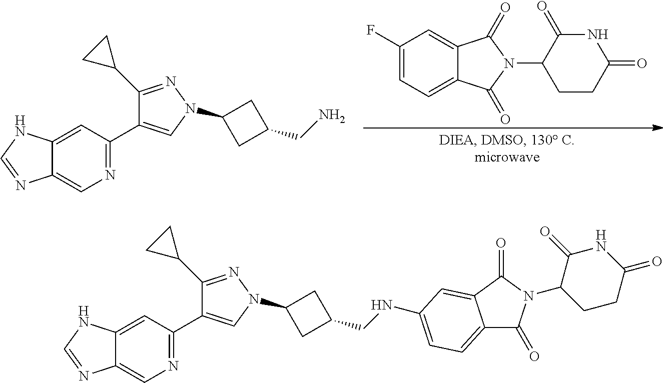 [SMILES: NC[C@H]1C[C@H](n2cc(-c3cc4[nH]cnc4cn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cc7[nH]cnc7cn6)c(C6CC6)n5)C4)cc3C2=O)C(=O)N1]

GS-308 was synthesized following the similar procedure for preparing GS-278 (10.6 mg, yield: 27.6%) MS (ESI) m/z=565.7 [M+H]⁺.

### Example 266. 5-(((trans-3-(3-Cyclopropyl-4-(5,6,7,8-tetrahydroquinolin-2-yl)-1H-pyrazol-1-yl)cyclobutyl)methyl)amino)-2-(2,6-dioxopiperidin-3-yl)isoindoline-1,3-dione (GS-308)

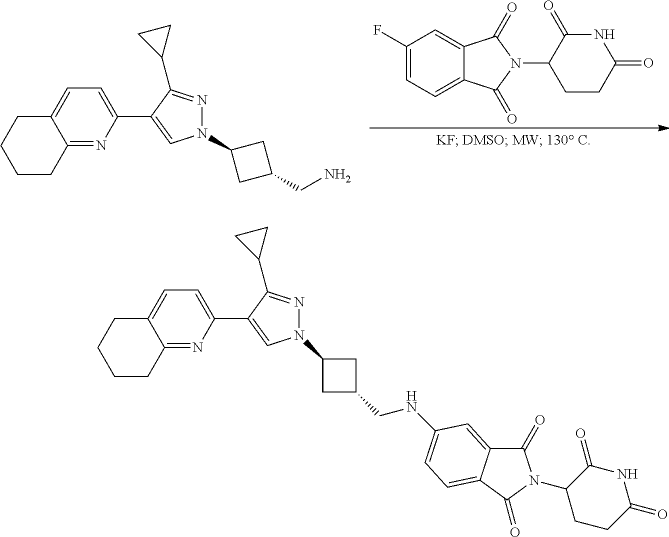 [SMILES: NC[C@H]1C[C@H](n2cc(-c3ccc4c(n3)CCCC4)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6ccc7c(n6)CCCC7)c(C6CC6)n5)C4)cc3C2=O)C(=O)N1]

GS-308 was synthesized following the similar procedure for preparing GS-278 (1.4 mg, yield: 7.8%). MS (ESI) m/z=579.8 [M+H]⁺.

### Example 267. 5-(((trans-3-(3-Cyclopropyl-4-(pyrido[3,4-b]pyrazin-5-yl)-1H-pyrazol-1-yl)cyclobutyl)methyl)amino)-2-(2,6-dioxopiperidin-3-yl)isoindoline-1,3-dione (GS-309)

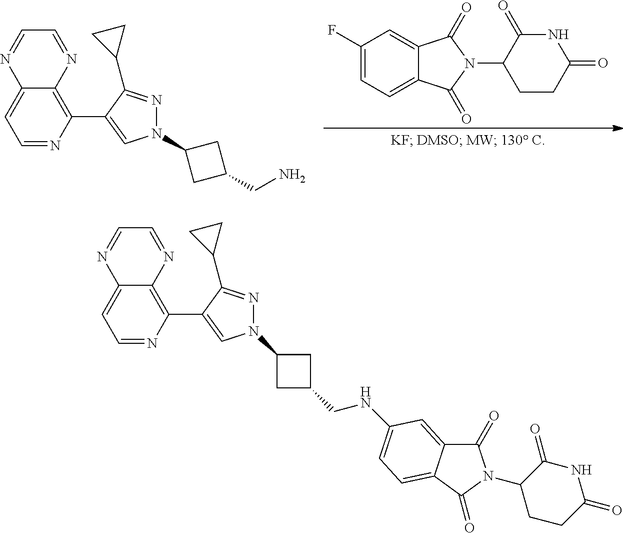

GS-309 was synthesized following the similar procedure for preparing GS-278 (2 mg, yield: 7.4%). MS (ESI) m/z=577.7 [M+H]⁺.

### Example 268. 5-(((trans-3-(3-Cyclopropyl-4-(1H-pyrazolo[3,4-c]pyridin-7-yl)-1H-pyrazol-1-yl)cyclobutyl)methyl)amino)-2-(2,6-dioxopiperidin-3-yl)isoindoline-1,3-dione (GS-310)

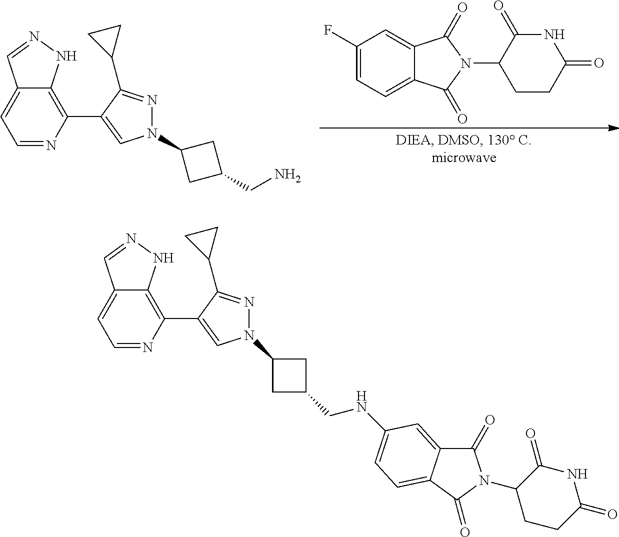 [SMILES: NC[C@H]1C[C@H](n2cc(-c3nccc4cn[nH]c34)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6nccc7cn[nH]c67)c(C6CC6)n5)C4)cc3C2=O)C(=O)N1]

GS-310 was synthesized following the similar procedure for preparing GS-278 (1 mg, yield: 5.5%). MS (ESI) m/z=565.6 [M+H]⁺.

### Example 269. 5-((6-(3-(4-(8-Bromoquinoxalin-2-yl)-1H-pyrazol-1-yl)azetidin-1-yl)-6-oxohexyl)amino)-2-(2,6-dioxopiperidin-3-yl)isoindoline-1,3-dione (GS-062)

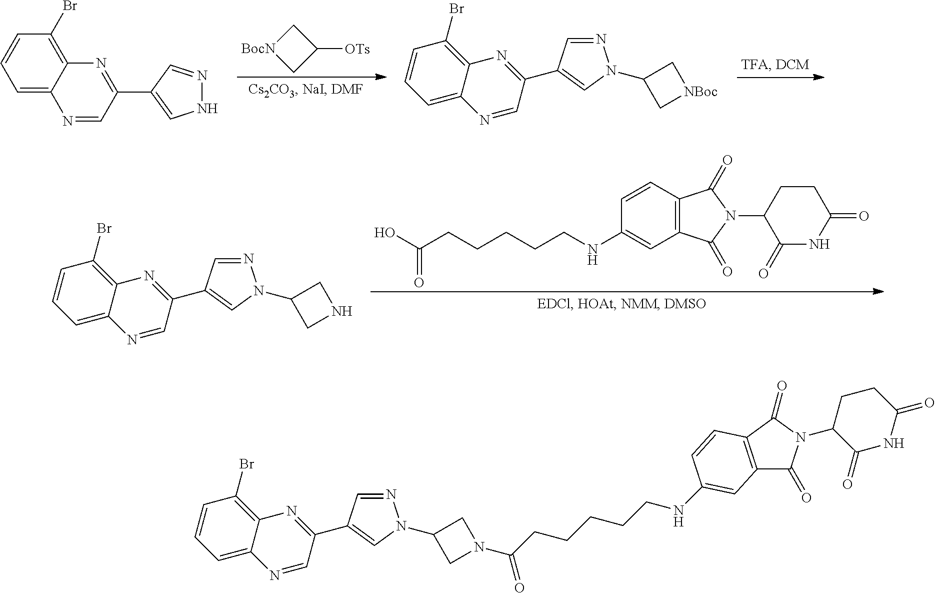 [SMILES: Brc1cccc2ncc(-c3cn[nH]c3)nc12 ; Cc1ccc(S(=O)(=O)OC2CN(C(=O)OC(C)(C)C)C2)cc1 ; CC(C)(C)OC(=O)N1CC(n2cc(-c3cnc4cccc(Br)c4n3)cn2)C1 ; Brc1cccc2ncc(-c3cnn(C4CNC4)c3)nc12 ; O=C(O)CCCCCNc1ccc2c(c1)C(=O)N(C1CCC(=O)NC1=O)C2=O ; O=C1CCC(N2C(=O)c3ccc(NCCCCCC(=O)N4CC(n5cc(-c6cnc7cccc(Br)c7n6)cn5)C4)cc3C2=O)C(=O)N1]

Step 1. Synthesis of tert-butyl 3-(4-(8-bromoquinoxalin-2-yl)-1H-pyrazol-1-yl)azetidine-1-carboxylate

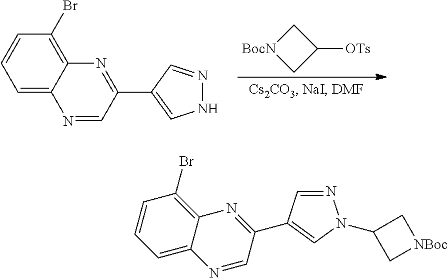

To a solution of 8-bromo-2-(1H-pyrazol-4-yl)quinoxaline (56 mg, 0.20 mmol) and tert-butyl 3-(tosyloxy)azetidine-1-carboxylate (65 mg, 0.20 mmol) in DMF (3 mL) was added Cs₂CO₃ (130 mg, 0.40 mmol) and NaI (12 mg, 0.08 mmol) at rt. Then the reaction mixture was stirred at 80° C. for 4 h. The mixture was purified by reverse-phase chromatography to provide the desired product (40 mg, 46% yield) as a light-yellow solid. MS (ESI) m/z=430.1 [M+H]⁺.

Step 2. Synthesis of 2-(1-(azetidin-3-yl)-1H-pyrazol-4-yl)-8-bromoquinoxaline

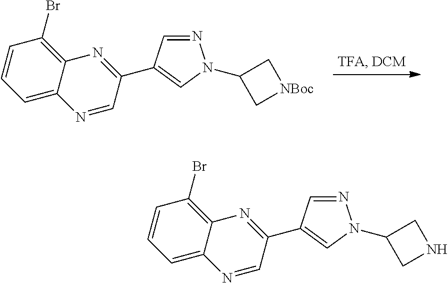 [SMILES: CC(C)(C)OC(=O)N1CC(n2cc(-c3cnc4cccc(Br)c4n3)cn2)C1 ; Brc1cccc2ncc(-c3cnn(C4CNC4)c3)nc12]

A solution of tert-butyl 3-(4-(8-bromoquinoxalin-2-yl)-1H-pyrazol-1-yl)azetidine-1-carboxylate (40 mg, 0.09 mmol) in TFA/DCM (1:1, 4 mL) was stirred at rit for 1 h. The resulting mixture was concentrated to provide the crude product (26 mg, 87% yield) as a light-yellow solid. This compound was used directly in the next step without further purification. MS (ESI) m/z=330.1 [M+H]⁺.

Step 3. Synthesis of 5-((6-(3-(4-(8-bromoquinoxalin-2-yl)-1H-pyrazol-1-yl)azetidin-1-yl)-6-oxohexyl)amino)-2-(2,6-dioxopiperidin-3-yl)isoindoline-1,3-dione

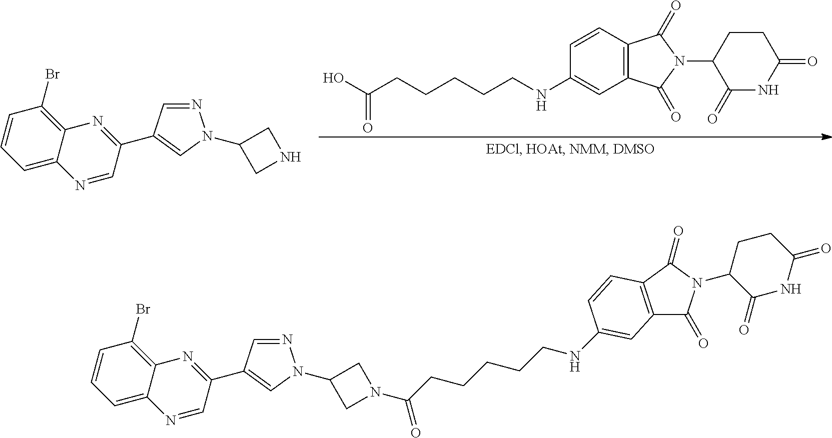 [SMILES: Brc1cccc2ncc(-c3cnn(C4CNC4)c3)nc12 ; O=C(O)CCCCCNc1ccc2c(c1)C(=O)N(C1CCC(=O)NC1=O)C2=O ; O=C1CCC(N2C(=O)c3ccc(NCCCCCC(=O)N4CC(n5cc(-c6cnc7cccc(Br)c7n6)cn5)C4)cc3C2=O)C(=O)N1]

A mixture of 6-((2-(2,6-dioxopiperidin-3-yl)-1,3-dioxoisoindolin-5-yl)amino)hexanoic acid (12 mg, 0.03 mmol), 2-(1-(azetidin-3-yl)-1H-pyrazol-4-yl)-8-bromoquinoxaline (10 mg, 0.03 mmol), EDCI (18 mg, 0.09 mmol), HOAt (13 mg, 0.09 mmol) and NMM (30 mg, 0.30 mmol) in DMSO (2 mL) was stirred at rt for 16 h. The reaction mixture was poured into water (10 mL) and extracted with ethyl acetate (3×15 mL). The combined organic layers were washed with saturated brine (20 mL), dried over anhydrous sodium sulfate, filtered, and concentrated under reduced pressure. The resulting residue was purified by reverse-phase chromatography to provide the desired product (8 mg, 44% yield) as a light-yellow solid. MS (ESI) m/z=699.2 [M+H]⁺.

### Example 270. 5-((6-(4-(4-Cyclopropyl-1H-pyrazol-1-yl)piperidin-1-yl)-6-oxohexyl)amino)-2-(2,6-dioxopiperidin-3-yl)isoindoline-1,3-dione (GS-064)

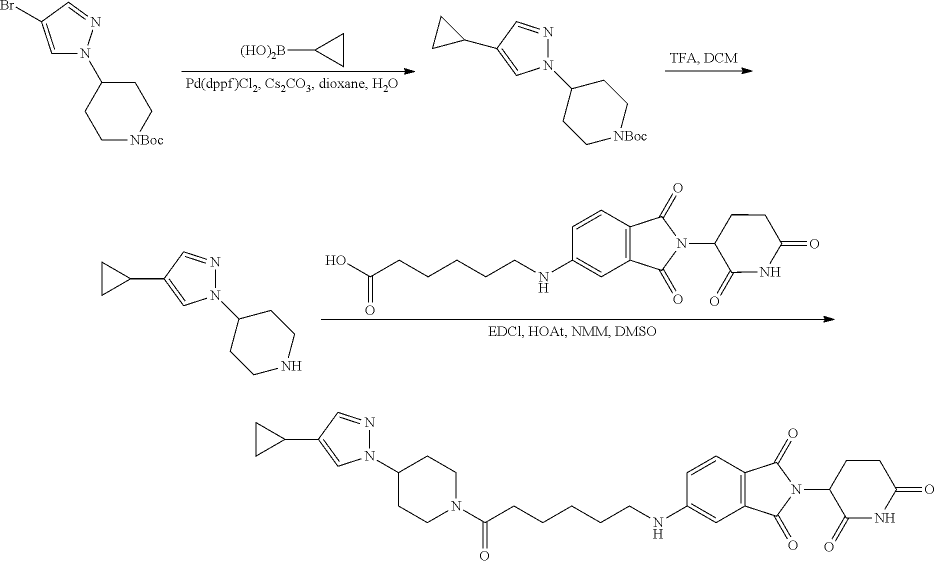 [SMILES: CC(C)(C)OC(=O)N1CCC(n2cc(Br)cn2)CC1 ; OB(O)C1CC1 ; CC(C)(C)OC(=O)N1CCC(n2cc(C3CC3)cn2)CC1 ; c1nn(C2CCNCC2)cc1C1CC1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(n5cc(C6CC6)cn5)CC4)cc3C2=O)C(=O)N1]

Step 1. Synthesis of tert-butyl 4-(4-cyclopropyl-1H-pyrazol-1-yl)piperidine-1-carboxylate

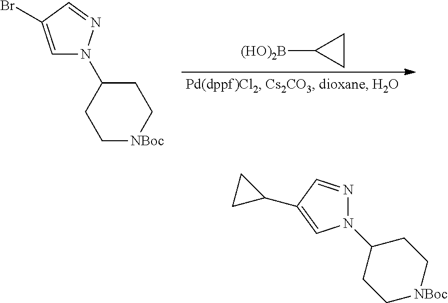 [SMILES: CC(C)(C)OC(=O)N1CCC(n2cc(Br)cn2)CC1 ; OB(O)C1CC1 ; CC(C)(C)OC(=O)N1CCC(n2cc(C3CC3)cn2)CC1]

To a solution of tert-butyl 4-(4-bromo-1H-pyrazol-1-yl)piperidine-1-carboxylate (100 mg, 0.3 mmol) in dioxane (10 mL) and H₂O (1 mL) was added cyclopropylboronic acid (40.1 mg, 0.46 mmol), Pd(dppf)Cl₂ (11.7 mg, 0.015 mmol) and Cs₂CO₃ (196 mg, 0.6 mmol) at rt. Then the mixture was stirred at 100° C. under inert atmosphere and microwave irradiation for 2 h. The mixture was concentrated and purified by reverse-phase chromatography to provide the desired product (22.4 mg, 25% yield) as a light-yellow solid. MS (ESI) m/z=292.3 [M+H]⁺.

The remaining steps were performed according to the standard procedures for preparing GS-062 to provide the desired product (2.7 mg, 40% yield) as a light-yellow solid. MS (ESI) m/z=561.3 [M+H]⁺.

### Example 271. 5-((6-(4-(8-Bromoquinoxalin-2-yl)piperidin-1-yl)-6-oxohexyl)amino)-2-(2,6-dioxopiperidin-3-yl)isoindoline-1,3-dione (GS-060)

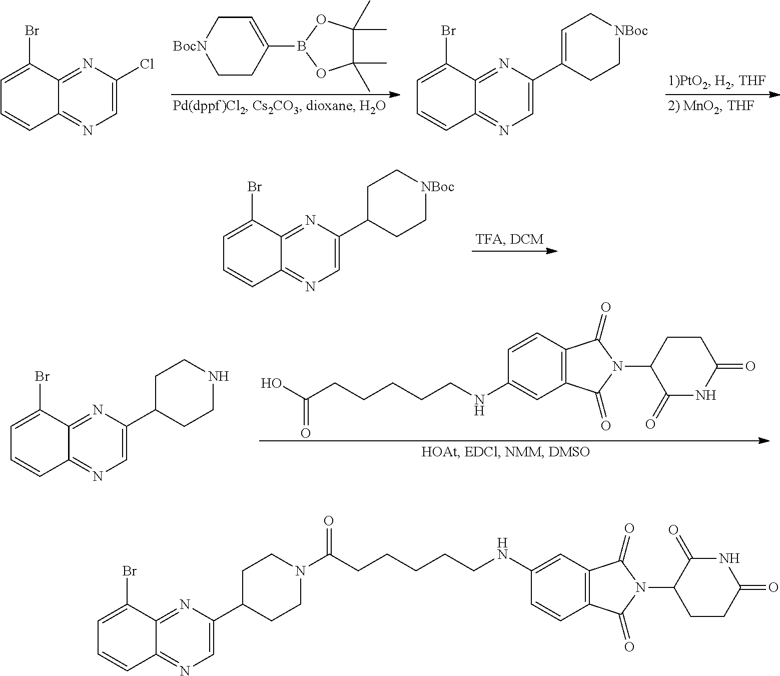

Step 1. Synthesis of tert-butyl 4-(8-bromoquinoxalin-2-yl)-3,6-dihydropyridine-1(2H)-carboxylate

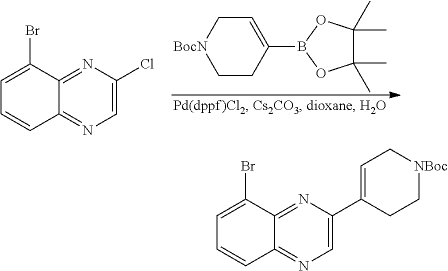 [SMILES: Clc1cnc2cccc(Br)c2n1 ; CC(C)(C)OC(=O)N1CC=C(B2OC(C)(C)C(C)(C)O2)CC1 ; CC(C)(C)OC(=O)N1CC=C(c2cnc3cccc(Br)c3n2)CC1]

To a solution of 8-bromo-2-chloroquinoxaline (100 mg, 0.4 mmol) in dioxane (5 mL) and H₂O (2 mL) were added tert-butyl 4-(4,4,5,5-tetramethyl-1,3,2-dioxaborolan-2-yl)-3,6-dihydropyridine-1(2H)-carboxylate (162 mg, 0.52 mmol), Pd(dppf)Cl₂ (15.2 mg, 0.02 mmol) and Cs₂CO₃ (196 mg, 0.6 mmol) at rt. Then the mixture was stirred at 100° C. under inert atmosphere for 2 h. The mixture was filtered, concentrated and purified by reverse-phase chromatography to provide the desired product (50.1 mg, 31% yield) as a light-yellow solid. MS (ESI) m/z=390.2 [M+H]⁺.

Step 2. Synthesis of tert-butyl 4-(8-bromoquinoxalin-2-yl)piperidine-1-carboxylate

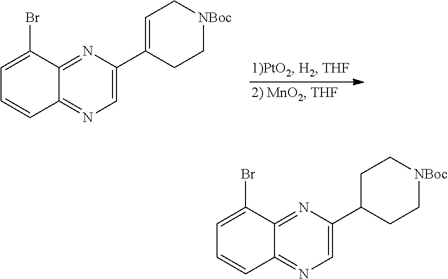 [SMILES: CC(C)(C)OC(=O)N1CC=C(c2cnc3cccc(Br)c3n2)CC1 ; CC(C)(C)OC(=O)N1CCC(c2cnc3cccc(Br)c3n2)CC1]

To a solution of tert-butyl 4-(8-bromoquinoxalin-2-yl)-3,6-dihydropyridine-1(2H)-carboxylate (20.0 mg, 0.19 mmol) in THF (10 mL) was added PtO₂ (10 mg). The reaction was stirred at rt under hydrogen balloon for 4 h. Then MnO₂ (9.8 mg) was added and the solution was stirred at rt under air for another 1 h. The reaction mixture was filtered, concentrated and purified by reverse-phase chromatography to provide the desired product (10.3 mg, 50% yield) as a light-yellow solid. MS (ESI) m/z=392.2 [M+H]⁺.

The remaining steps were performed according to the standard procedures for preparing GS-062 to provide the desired product (1.4 mg, 15% yield) as a light-yellow solid. MS (ESI) m/z=661.2 [M+H]⁺.

### Example 272. 5-((6-(4-(4-(Azetidin-1-yl)-1H-pyrazol-1-yl)piperidin-1-yl)-6-oxohexyl)amino)-2-(2,6-dioxopiperidin-3-yl)isoindoline-1,3-dione (GS-063)

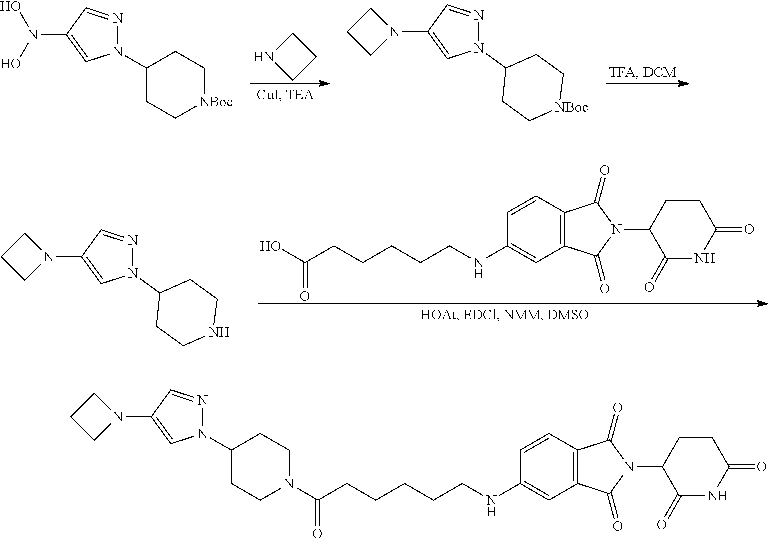

Step 1. Synthesis of tert-butyl 4-(4-(azetidin-1-yl)-1H-pyrazol-1-yl)piperidine-1-carboxylate

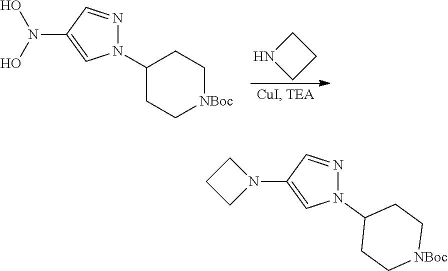

To a solution of (1-(1-(tert-butoxycarbonyl)piperidin-4-yl)-1H-pyrazol-4-yl)boronic acid (90 mg, 0.30 mmol) and azetidine (18 mg, 0.30 mmol) in DMF (3 mL) was added CuI (18 mg, 0.09 mmol) and TEA (60 mg, 0.60 mmol) at rt. Then the reaction mixture was stirred in the air for 16 h. The mixture was purified by reverse-phase chromatography to provide the desired product (20 mg, 22% yield) as a light-yellow solid. MS (ESI) m/z=307.2 [M+H]⁺.

The remaining steps were performed according to the standard procedures for preparing GS-062 to provide the desired product (10 mg, 27% yield) as a light-yellow solid. MS (ESI) m/z=576.3 [M+H]⁺.

### Example 273. 5-((6-(4-(8-Bromoquinoxalin-2-yl)-1H-pyrazol-1-yl)hexyl)amino)-2-(2,6-dioxopiperidin-3-yl)isoindoline-1,3-dione (GS-315)

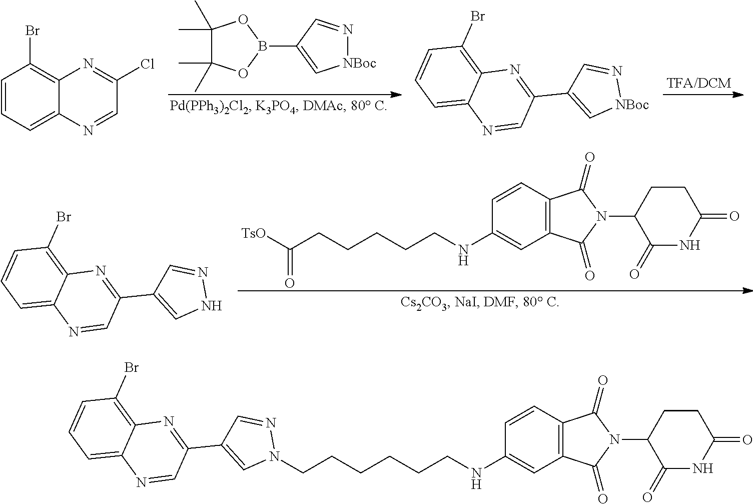

Step 1. Synthesis of tert-butyl 4-(8-bromoquinoxalin-2-yl)-1H-pyrazole-1-carboxylate

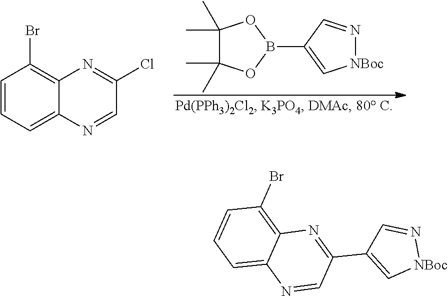

A mixture of 8-bromo-2-chloroquinoxaline (150 mg, 0.62 mmol), tert-butyl 4-(4,4,5,5-tetramethyl-1,3,2-dioxaborolan-2-yl)-1H-pyrazole-1-carboxylate (182 mg, 0.62 mmol), Pd(dppf)Cl₂ (23 mg, 0.031 mmol) and K₃PO₄ (410 mg, 1.55 mmol) in DMAc (8 mL) was stirred at 80° C. under N₂ atmosphere for 3 h. The solution was poured into water (40 mL) and extracted with ethyl acetate (3×20 mL). The combined organic layers were washed with saturated brine (40 mL), dried over anhydrous sodium sulfate, filtered, and concentrated under reduced pressure. The resulting residue was purified by column chromatography on silica gel to provide the desired product (169 mg, 73% yield) as a light-yellow solid. MS (ESI) m/z=375.0 [M+H]⁺.

Step 2. Synthesis of 8-bromo-2-(1H-pyrazol-4-yl)quinoxaline

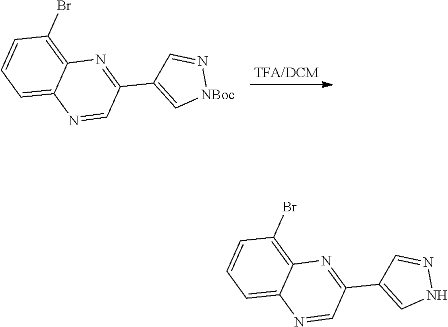 [SMILES: CC(C)(C)OC(=O)n1cc(-c2cnc3cccc(Br)c3n2)cn1 ; Brc1cccc2ncc(-c3cn[nH]c3)nc12]

A mixture of tert-butyl 4-(8-bromoquinoxalin-2-yl)-1H-pyrazole-1-carboxylate (60 mg, 0.16 mmol) in TFA/DCM (1:1, 4 mL) was stirred at rt for 1 h. The resulting mixture was concentrated to provide the crude product (40 mg, 91% yield) as a light-yellow solid which was used directly in the next step without further purification. MS (ESI) m/z=275.0 [M+H]⁺.

Step 3. Synthesis of 5-((6-(4-(8-bromoquinoxalin-2-yl)-1H-pyrazol-1-yl)hexyl)amino)-2-(2,6-dioxopiperidin-3-yl)isoindoline-1,3-dione

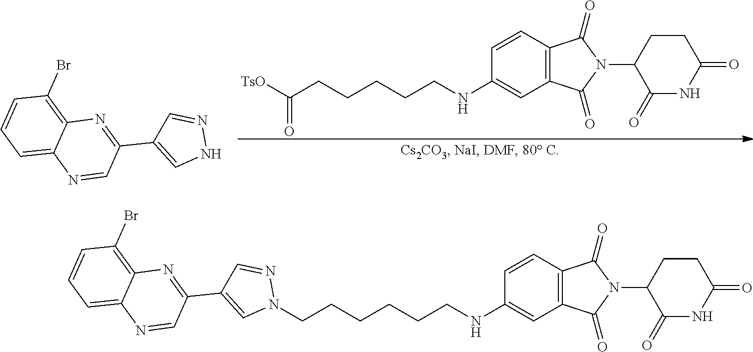

A mixture of 8-bromo-2-(1H-pyrazol-4-yl)quinoxaline (10 mg, 0.036 mmol), 6-((2-(2,6-dioxopiperidin-3-yl)-1,3-dioxoisoindolin-5-yl)amino)hexyl 4-methylbenzene sulfonate (23 mg, 0.043 mmol), Cs₂CO₃ (23 mg, 0.072 mmol) and NaI (6 mg, 0.036 mmol) in DMF (3 mL) was stirred at 80° C. for 1 h. The reaction mixture was poured into water (15 mL) and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (8.4 mg, 37% yield) as a light-yellow solid. MS (ESI) m/z=630.2 [M+H]⁺.

### Example 274. N-(3-((2-(2,6-Dioxopiperidin-3-yl)-1,3-dioxoisoindolin-5-yl)amino)propyl)-2-(4-(4-(quinoxalin-2-yl)-1H-pyrazol-1-yl)piperidin-1-yl)acetamide (GS-316)

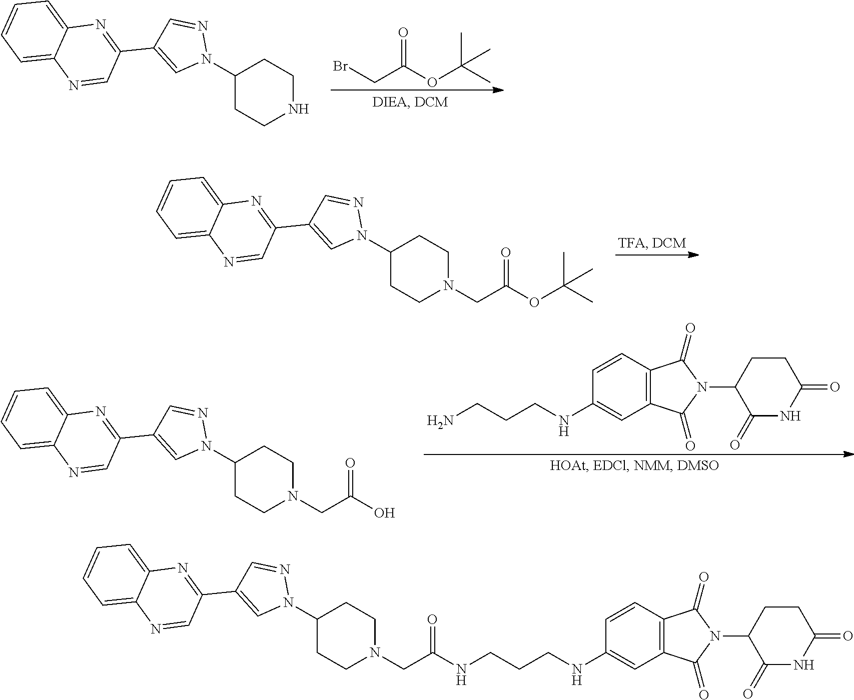

Step 1. Synthesis of tert-butyl 2-(4-(4-(quinoxalin-2-yl)-1H-pyrazol-1-yl)piperidin-1-yl)acetate

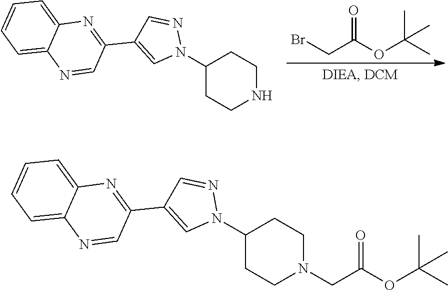

To a solution of 2-(1-(piperidin-4-yl)-1H-pyrazol-4-yl)quinoxaline (56 mg, 0.20 mmol) and tert-butyl 2-bromoacetate (39 mg, 0.20 mmol) in DMSO (2 mL) was added DIEA (42 mg, 0.40 mmol) at rt. The reaction mixture was stirred at 80° C. for 4 h, before it was purified by reverse-phase chromatography to provide the desired product (48 mg, 60% yield) as a light-yellow solid. MS (ESI) m/z=394.3 [M+H]⁺.

Step 2. Synthesis of 2-(4-(4-(quinoxalin-2-yl)-1H-pyrazol-1-yl)piperidin-1-yl)acetic acid

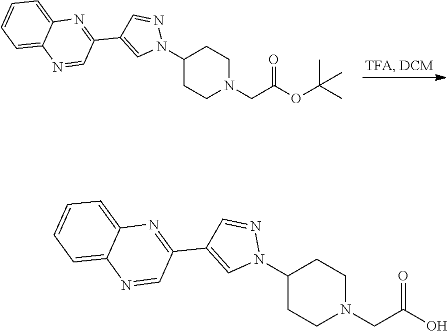 [SMILES: CC(C)(C)OC(=O)CN1CCC(n2cc(-c3cnc4ccccc4n3)cn2)CC1 ; O=C(O)CN1CCC(n2cc(-c3cnc4ccccc4n3)cn2)CC1]

A mixture of tert-butyl 2-(4-(4-(quinoxalin-2-yl)-1H-pyrazol-1-yl)piperidin-1-yl)acetate (48 mg, 0.12 mmol) in DCM (2 mL) and TFA (2 mL) was stirred at rt for 1 h. The resulting mixture was concentrated to provide the crude product (32 mg, 78% yield) as a light-yellow solid. This compound was used directly in the next step without further purification. MS (ESI) m/z=236.2 [M−H]⁻.

Step 3. Synthesis of N-(3-((2-(2,6-dioxopiperidin-3-yl)-1,3-dioxoisoindolin-5-yl)amino)propyl)-2-(4-(4-(quinoxalin-2-yl)-1H-pyrazol-1-yl)piperidin-1-yl)acetamide

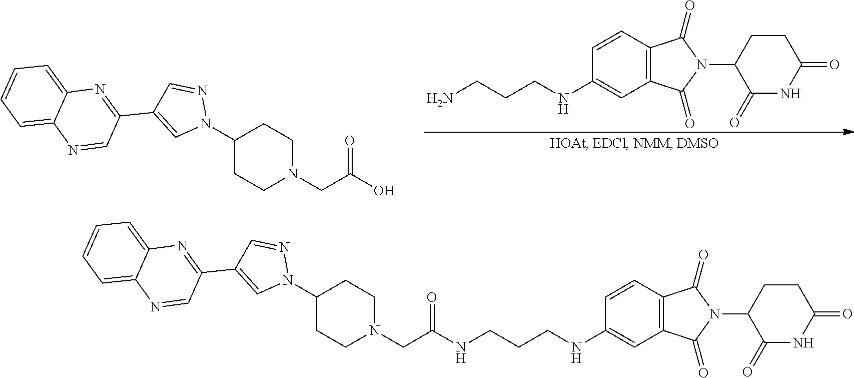 [SMILES: O=C(O)CN1CCC(n2cc(-c3cnc4ccccc4n3)cn2)CC1 ; NCCCNc1ccc2c(c1)C(=O)N(C1CCC(=O)NC1=O)C2=O ; O=C(CN1CCC(n2cc(-c3cnc4ccccc4n3)cn2)CC1)NCCCNc1ccc2c(c1)C(=O)N(C1CCC(=O)NC1=O)C2=O]

A mixture of 2-(4-(4-(quinoxalin-2-yl)-1H-pyrazol-1-yl)piperidin-1-yl)acetic acid (32 mg, 0.09 mmol), 5-((3-aminopropyl)amino)-2-(2,6-dioxopiperidin-3-yl)isoindoline-1,3-dione (33 mg, 0.10 mmol), EDCI (39 mg, 0.18 mmol), HOAt (26 mg, 0.18 mmol) and NMM (45 mg, 0.45 mmol) in DMSO (2 mL) was stirred at rt for 16 h. The reaction mixture was poured into water (10 mL) and extracted with ethyl acetate (10 mL×3). The combined organic layers were washed with saturated brine (10 mL), dried over anhydrous sodium sulfate, filtered, and concentrated under reduced pressure. The resulting residue was purified by reverse-phase chromatography to provide the desired product (12 mg, 20% yield) as a light-yellow solid. MS (ESI) m/z=650.4 [M+H]⁺.

### Example 275. N-(3-((2-(2,6-Dioxopiperidin-3-yl)-1,3-dioxoisoindolin-5-yl)amino)propyl)-2-(4-(4-(quinolin-2-yl)-1H-pyrazol-1-yl)piperidin-1-yl)acetamide (GS-317)

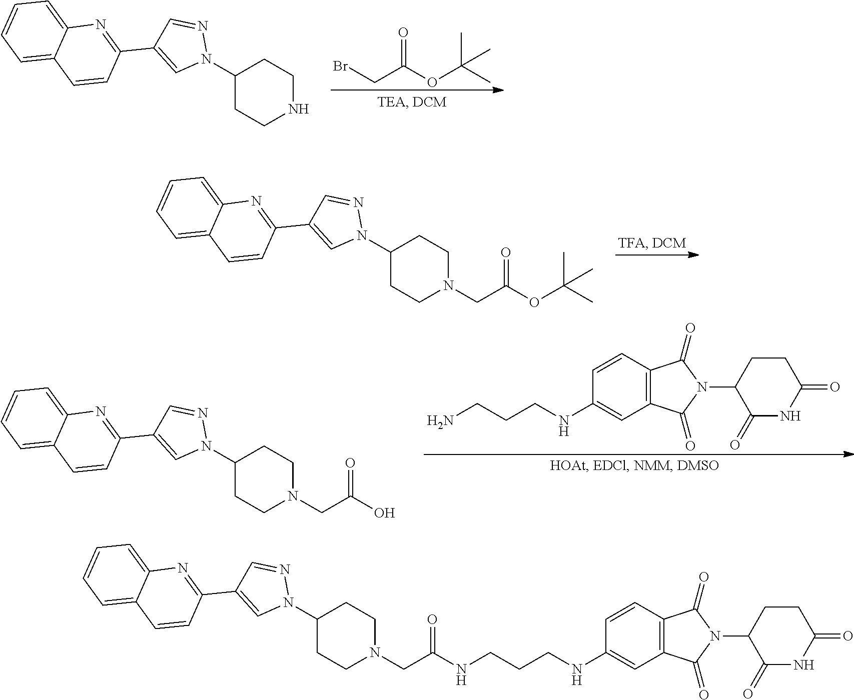

GS-317 was synthesized following the standard procedure for preparing GS-316 (2.0 mg, 26% yield) as a light-yellow solid. MS (ESI) m/z=649.3 [M+H]⁺.

### Example 276. 2-(2,6-Dioxopiperidin-3-yl)-5-((4-oxo-4-(4-(pyridin-2-yl)piperazin-1-yl)butyl)amino)isoindoline-1,3-dione (GS-318)

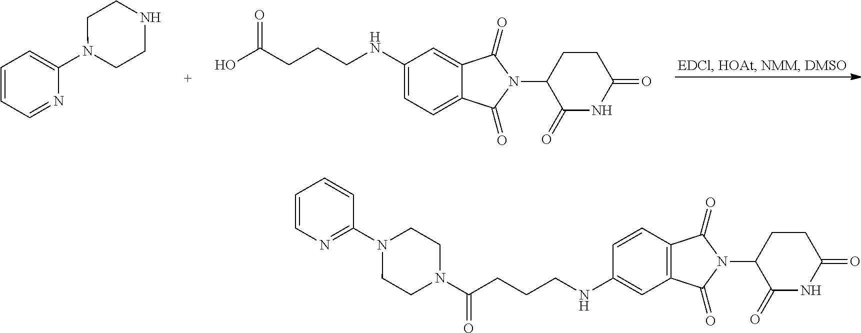 [SMILES: c1ccc(N2CCNCC2)nc1 ; O=C(O)CCCNc1ccc2c(c1)C(=O)N(C1CCC(=O)NC1=O)C2=O ; O=C1CCC(N2C(=O)c3ccc(NCCCC(=O)N4CCN(c5ccccn5)CC4)cc3C2=O)C(=O)N1]

GS-318 was synthesized following the standard procedure for preparing GS-062 (7.5 mg, 51% yield) as a light-yellow solid. MS (ESI) m/z=505.3 [M+H]⁺.

### Example 277. 2-(2,6-Dioxopiperidin-3-yl)-5-((4-oxo-4-(4-phenylpiperazin-1-yl)butyl)amino)isoindoline-1,3-dione (GS-319)

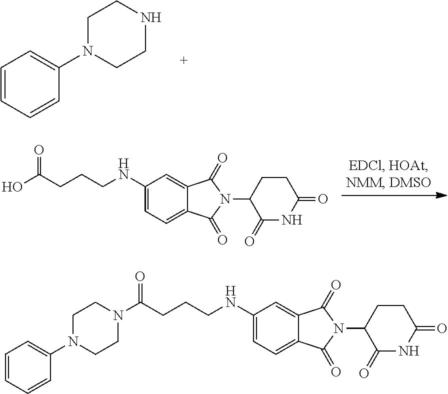

GS-319 was synthesized following the standard procedure for preparing GS-062 (8.2 mg, 45% yield) as a light-yellow solid. MS (ESI) m/z=504.3 [M+H]⁺.

### Example 278. N-(2-((2-(2,6-Dioxopiperidin-3-yl)-1,3-dioxoisoindolin-5-yl)amino)ethyl)-2-(4-(methyl(pyridin-2-yl)amino)piperidin-1-yl)acetamide (GS-320)

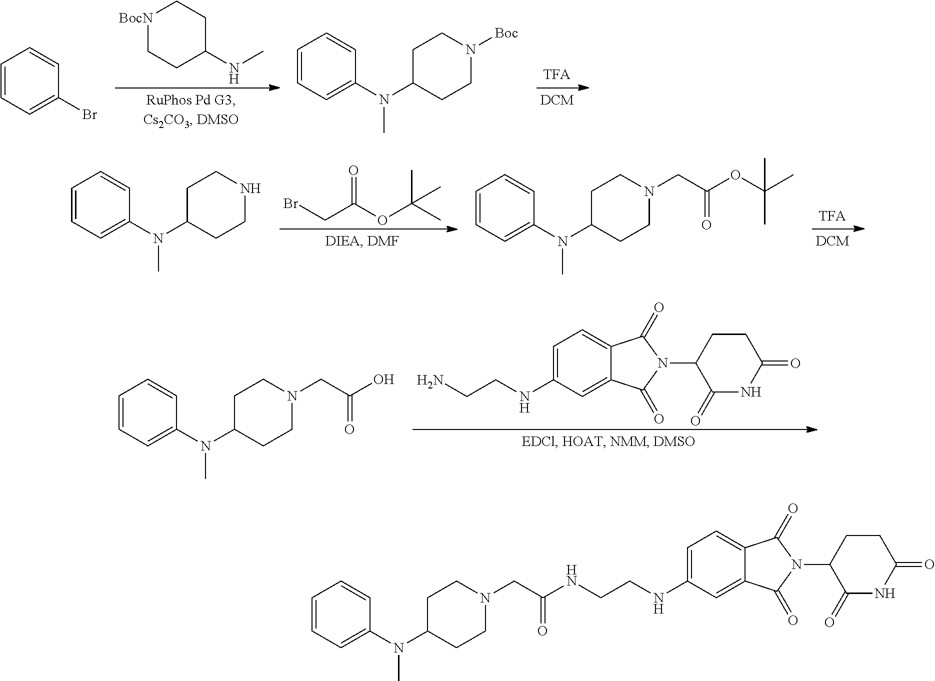

GS-320 was synthesized following the standard procedures for preparing GS-321 (5.3 mg, 25% yield) as a yellow solid. MS (ESI) m/z=547.3 [M+H]⁺.

### Example 279. N-(2-((2-(2,6-Dioxopiperidin-3-yl)-1,3-dioxoisoindolin-5-yl)amino)ethyl)-2-(4-(methyl(pyridin-2-yl)amino)piperidin-1-yl)acetamide (GS-321)

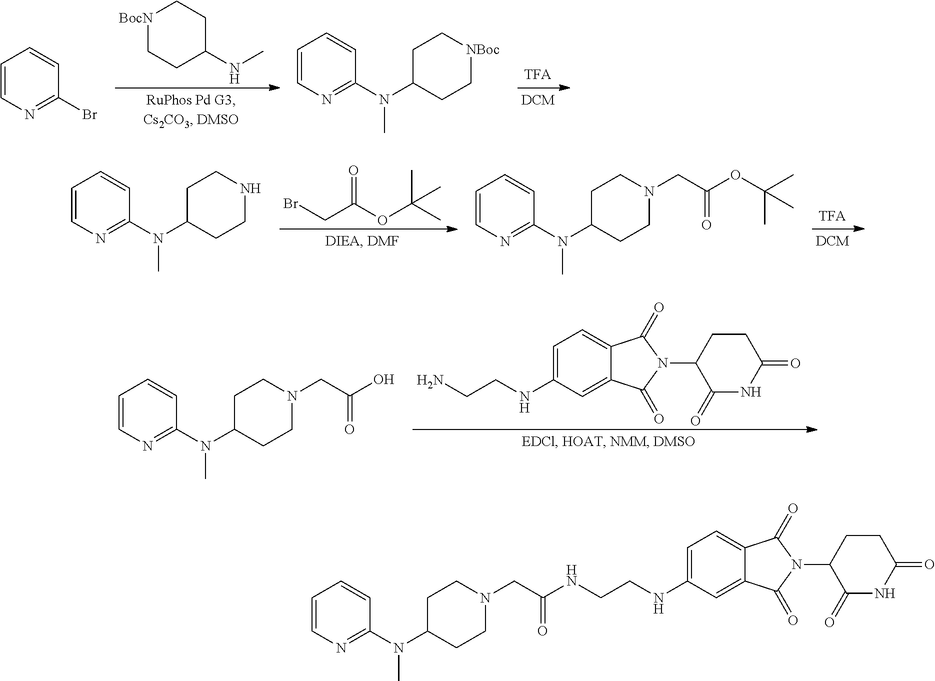

Step 1. Synthesis of tert-butyl 4-(methyl(pyridin-2-yl)amino)piperidine-1-carboxylate

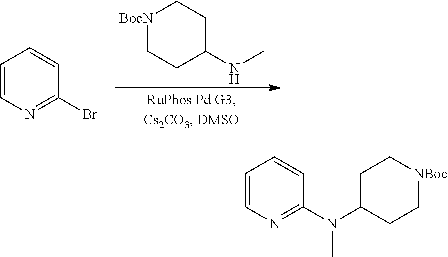 [SMILES: Brc1ccccn1 ; CNC1CCN(C(=O)OC(C)(C)C)CC1 ; CN(c1ccccn1)C1CCN(C(=O)OC(C)(C)C)CC1]

To a solution of 2-bromopyridine (80 mg, 0.51 mmol) and tert-butyl 4-(methylamino)piperidine-1-carboxylate (131 mg, 0.62 mmol) in DMSO (5 mL) was added Cs₂CO₃ (497 mg, 1.53 mmol) and Ruphos-Pd-G3 (32 mg, 0.05 mmol) at rt under N₂. After stirring at 100° C. for 16 h, the reaction mixture was purified by reverse-phase chromatography to provide the desired product (65 mg, 43% yield) as a white solid. MS (ESI) m/z=292.5 [M+H]⁺.

Step 2. Synthesis of N-methyl-N-(piperidin-4-yl)pyridin-2-amine

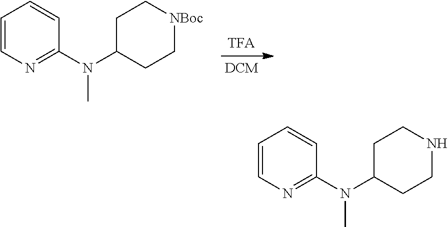 [SMILES: CN(c1ccccn1)C1CCN(C(=O)OC(C)(C)C)CC1 ; CN(c1ccccn1)C1CCNCC1]

To a solution of tert-butyl 4-(methyl(pyridin-2-yl)amino)piperidine-1-carboxylate (65 mg, 0.22 mmol) in DCM (2 mL) was added TFA (1 mL) at 0° C. The reaction mixture was stirred at rt for 1 h. The solvents were removed under vacuum. The resulting residue was used in the next step directly without further purification. MS (ESI) m/z=192.5 [M+H]⁺.

Step 3. Synthesis of tert-butyl 2-(4-(methyl(pyridin-2-yl)amino)piperidin-1-yl)acetate

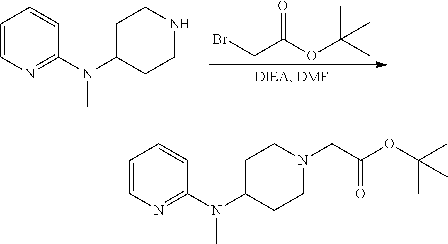

To a solution of N-methyl-N-(piperidin-4-yl)pyridin-2-amine (65 mg, 0.22 mmol) in DMF (2 mL) were added tert-butyl 2-bromoacetate (51 mg, 0.26 mmol) and DIEA (85 mg, 0.66 mmol) at 0° C. Then the reaction mixture was stirred at rt for 1 h. The solution was poured into water (10 mL) and extracted with EtOAc (3×10 mL). The combined organic layers were washed with saturated brine, dried over anhydrous sodium sulfate, filtered, and concentrated under reduced pressure. The resulting residue was purified by silica gel flash chromatography to provide the desired product (35 mg, 52% yield) as a yellow oil. MS (ESI) m/z=306.3 [M+H]⁺.

Step 4. Synthesis of 2-(4-(methyl(pyridin-2-yl)amino)piperidin-1-yl)acetic acid

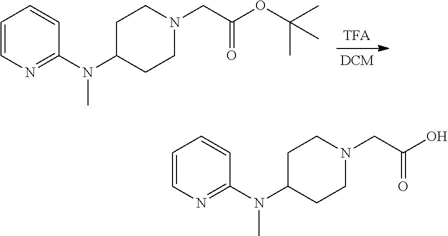 [SMILES: CN(c1ccccn1)C1CCN(CC(=O)OC(C)(C)C)CC1 ; CN(c1ccccn1)C1CCN(CC(=O)O)CC1]

To a solution of tert-butyl 2-(4-(methyl(pyridin-2-yl)amino)piperidin-1-yl)acetate (35 mg, 0.11 mmol) in DCM (2 mL) was added TFA (1 mL) at 0° C. After stirring at rt for 1 h, the reaction was concentrated under vacuum. The resulting residue was used in the next step directly without further purification. MS (ESI) m/z=250.1 [M+H]⁺.

Step 5. Synthesis of N-(2-((2-(2,6-dioxopiperidin-3-yl)-1,3-dioxoisoindolin-5-yl)amino)ethyl)-2-(4-(methyl(pyridin-2-yl)amino)piperidin-1-yl)acetamide

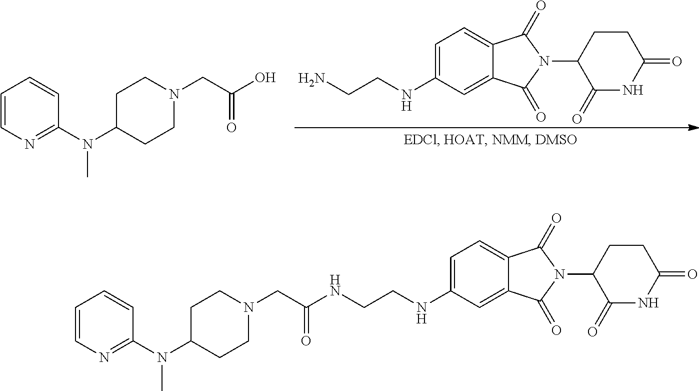 [SMILES: CN(c1ccccn1)C1CCN(CC(=O)O)CC1 ; NCCNc1ccc2c(c1)C(=O)N(C1CCC(=O)NC1=O)C2=O ; CN(c1ccccn1)C1CCN(CC(=O)NCCNc2ccc3c(c2)C(=O)N(C2CCC(=O)NC2=O)C3=O)CC1]

To a solution of 2-(4-(methyl(pyridin-2-yl)amino)piperidin-1-yl)acetic acid (30 mg, 0.11 mmol) in DMSO (2 mL) were added 5-((2-aminoethyl)amino)-2-(2,6-dioxopiperidin-3-yl)isoindoline-1,3-dione (34 mg, 0.11 mmol), EDCI (31 mg, 0.16 mmol), HOAT (22 mg, 0.33 mmol) and NMM (33 mg, 0.33 mmol) at rt. After stirring at rt for 16 h, The reaction was purified by reverse-phase chromatography, followed by prep-TLC to provide the desired product (21 mg, 35% yield) as a yellow solid. MS (ESI) m/z=548.3 [M+H]⁺.

### Example 280. N-(2-((2-(2,6-Dioxopiperidin-3-yl)-1,3-dioxoisoindolin-5-yl)amino)ethyl)-2-(4-(methyl(1H-pyrrolo[3,2-b]pyridin-5-yl)amino)piperidin-1-yl)acetamide (GS-322)

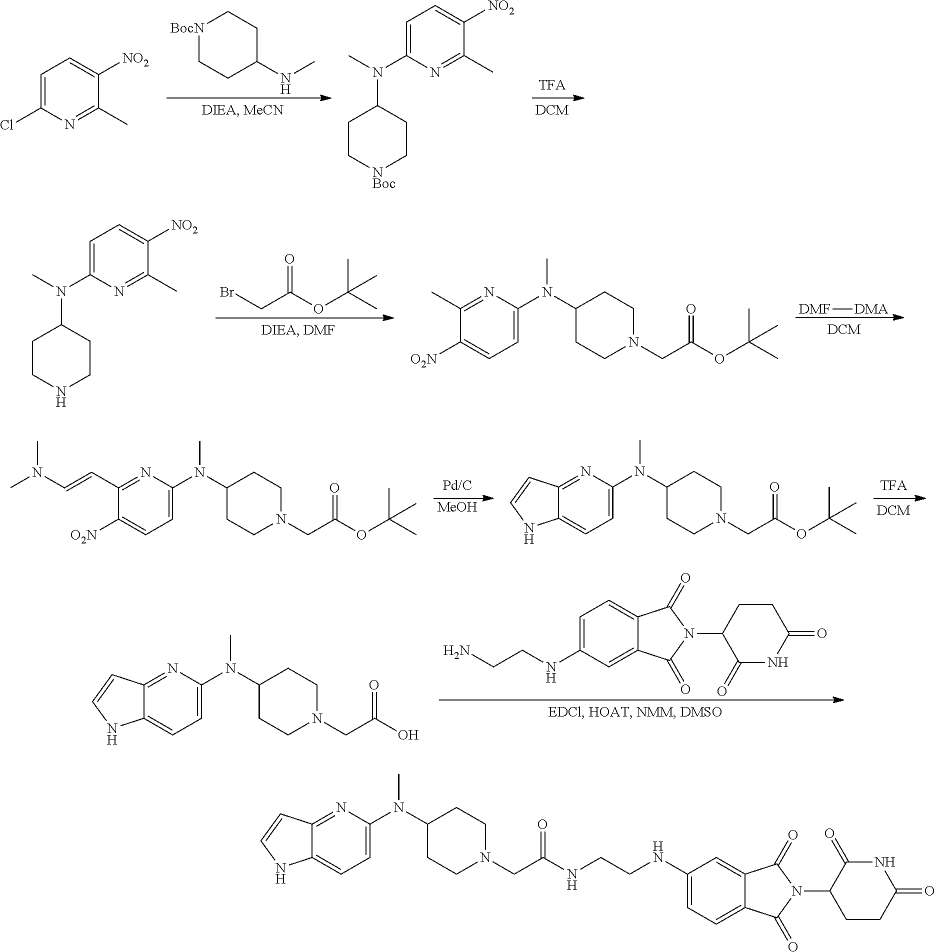

Step 1. Synthesis of tert-butyl 4-(methyl(6-methyl-5-nitropyridin-2-yl)amino)piperidine-1-carboxylate

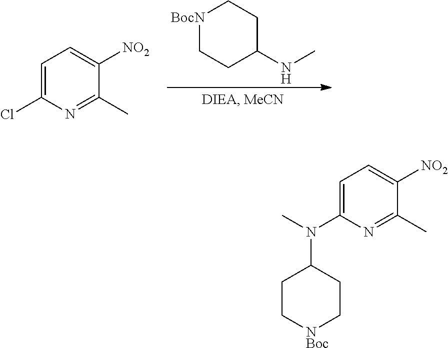 [SMILES: Cc1nc(Cl)ccc1[N+](=O)[O-] ; CNC1CCN(C(=O)OC(C)(C)C)CC1 ; Cc1nc(N(C)C2CCN(C(=O)OC(C)(C)C)CC2)ccc1[N+](=O)[O-]]

To a solution of 6-chloro-2-methyl-3-nitropyridine (2 g, 11.6 mmol) in MeCN (100 mL) were added tert-butyl 4-(methylamino)piperidine-1-carboxylate (5 g, 23.2 mmol) and DIEA (4.5 g, 34.8 mmol). The reaction mixture was stirred at 70° C. for 16 h. Then the solution was diluted with water (100 mL) and stirred for another 15 min. The precipitate was collected by filtration. The solid was washed with water and dried under reduced pressure to provide the desired product (2.3 g, 60% yield) as a yellow solid. MS (ESI) m/z=351.2 [M+H]⁺.

Step 2. Synthesis of N,6-dimethyl-5-nitro-N-(piperidin-4-yl)pyridin-2-amine

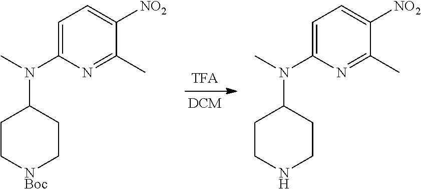 [SMILES: Cc1nc(N(C)C2CCN(C(=O)OC(C)(C)C)CC2)ccc1[N+](=O)[O-] ; Cc1nc(N(C)C2CCNCC2)ccc1[N+](=O)[O-]]

To a solution of tert-butyl 4-(methyl(6-methyl-5-nitropyridin-2-yl)amino)piperidine-1-carboxylate (500 mg, 1.42 mmol) in DCM (5 mL) was added TFA (2 mL) at 0° C. After stirring at rt for 1 h, the reaction was concentrated. The resulting residue was used in the next step directly without further purification. MS (ESI) m/z=251.2 [M+H]⁺.

Step 3. Synthesis of tert-butyl 2-(4-(methyl(6-methyl-5-nitropyridin-2-yl)amino)piperidin-1-yl)acetate

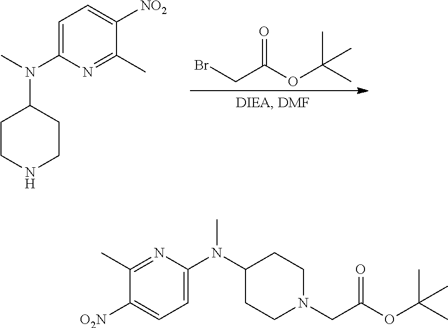

To a solution of N,6-dimethyl-5-nitro-N-(piperidin-4-yl)pyridin-2-amine (520 mg, 1.42 mmol) in DMF (5 mL) were added tert-butyl 2-bromoacetate (334 mg, 1.71 mmol) and DIEA (554 mg, 4.26 mmol) at 0° C. The reaction mixture was stirred at rt for 1 h. Then the solution was poured into water (20 mL) and extracted with ethyl acetate (3×20 mL). The combined organic layers were washed with saturated brine, dried over anhydrous sodium sulfate, filtered, and concentrated under reduced pressure. The resulting residue was purified by silica gel flash chromatography to provide the desired product (415 mg, 81% yield) as a yellow oil. MS (ESI) m/z=365.4 [M+H]⁺.

Step 4. Synthesis of tert-butyl (E)-2-(4-((6-(2-(dimethylamino)vinyl)-5-nitropyridin-2-yl)(methyl)amino)piperidin-1-yl)acetate

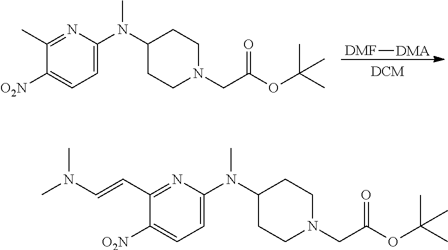

To a solution of tert-butyl 2-(4-(methyl(6-methyl-5-nitropyridin-2-yl)amino)piperidin-1-yl)acetate (415 mg, 1.14 mmol) in DMF (5 mL) was added DMF-DMA (271 mg, 2.28 mmol). The reaction mixture was stirred at 100° C. for 16 h, before it was quenched with water (15 mL). The resulting solution was extracted with ethyl acetate (3×20 mL), dried over anhydrous sodium sulfate, filtered, and concentrated to provide the desired product (380 mg, 80% yield) as a yellow oil. This product was used in the next step directly without further purification.

Step 5. Synthesis of tert-butyl 2-(4-(methyl(1H-pyrrolo[3,2-b]pyridin-5-yl)amino)piperidin-1-yl)acetate

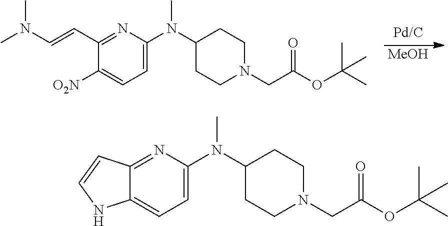

To a solution of tert-butyl (E)-2-(4-((6-(2-(dimethylamino)vinyl)-5-nitropyridin-2-yl)(methyl)amino)piperidin-1-yl)acetate (380 mg, 0.9 mmol) in MeOH (10 mL) was added 10% Pd/C (40 mg). The reaction mixture was stirred at rt for 16 h under hydrogen balloon. Then the solution was filtered, and concentrated. The resulting residue was purified by reverse-phase chromatography to provide the desired product (260 mg, 84% yield) as a white solid. MS (ESI) m/z=345.3 [M+H]⁺.

Step 6. Synthesis of 2-(4-(methyl(1H-pyrrolo[3,2-b]pyridin-5-yl)amino)piperidin-1-yl)acetic acid

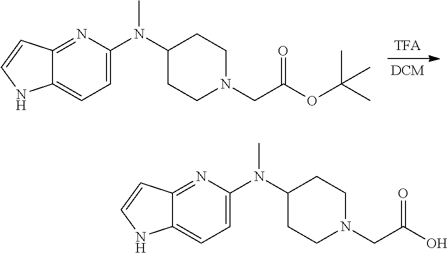

To a solution of tert-butyl 2-(4-(methyl(1H-pyrrolo[3,2-b]pyridin-5-yl)amino)piperidin-1-yl)acetate (15 mg, 0.04 mmol) in DCM (2 mL) was added TFA (1 mL) at 0° C. After stirring at rt for 1 h, the reaction was concentrated. The resulting residue was used in the next step directly without further purification. MS (ESI) m/z=289.2 [M+H]⁺.

Step 7. Synthesis of N-(2-((2-(2,6-dioxopiperidin-3-yl)-1,3-dioxoisoindolin-5-yl)amino)ethyl)-2-(4-(methyl(1H-pyrrolo[3,2-b]pyridin-5-yl)amino)piperidin-1-yl)acetamide

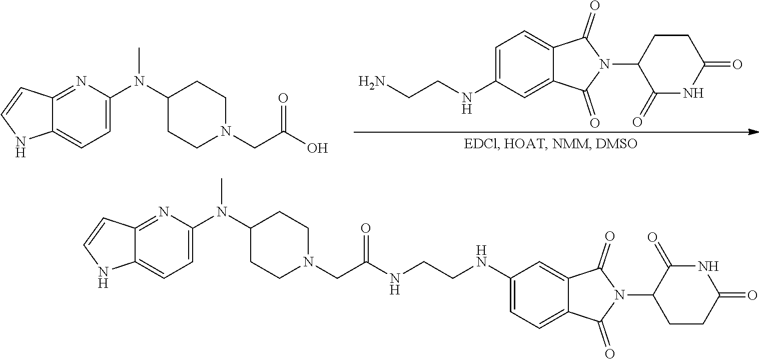

To a solution of 2-(4-(methyl(1H-pyrrolo[3,2-b]pyridin-5-yl)amino)piperidin-1-yl)acetic acid (15 mg, 0.04 mmol) in DMSO (1 mL) were added 5-((2-aminoethyl)amino)-2-(2,6-dioxopiperidin-3-yl)isoindoline-1,3-dione (17 mg, 0.06 mmol), EDCI (11 mg, 0.06 mmol), HOAT (8 mg, 0.06 mmol) and NMM (20 mg, 0. 3 mmol). After stirring at rt for 16 h, the reaction was purified by reverse-phase chromatography, followed by prep-TLC to provide the desired product (6.2 mg, 27% yield) as a yellow solid. MS (ESI) m/z=587.3 [M+H]⁺.

### Example 281. 2-(2,6-Dioxopiperidin-3-yl)-5-((5-oxo-5-(4-(4-(quinoxalin-2-yl)-1H-pyrazol-1-yl)piperidin-1-yl)pentyl)amino)isoindoline-1,3-dione (GS-323)

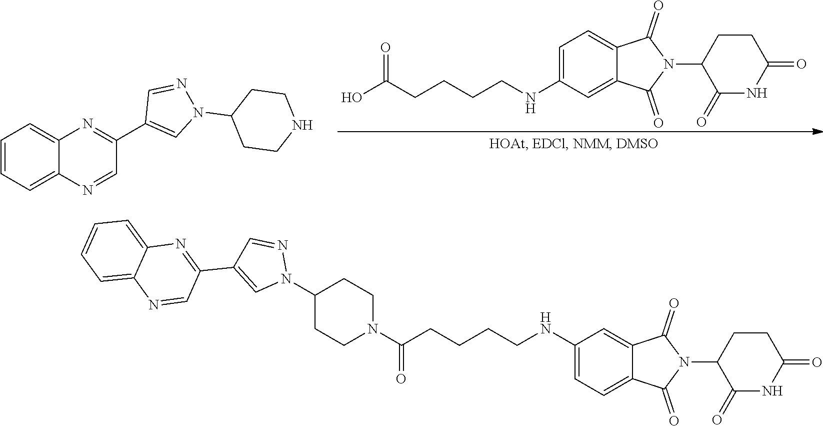

GS-323 was synthesized following the standard procedure or preparing GS-062 (5.5 mg, 30% yield) as a light-yellow solid. MS (ESI) m/z=635.4 [M+H]⁺.

### Example 282. 2-(2,6-Dioxopiperidin-3-yl)-5-((2-(3-oxo-3-(4-(4-(quinoxalin-2-yl)-1H-pyrazol-1-yl)piperidin-1-yl)propoxy)ethyl)amino)isoindoline-1,3-dione (GS-324)

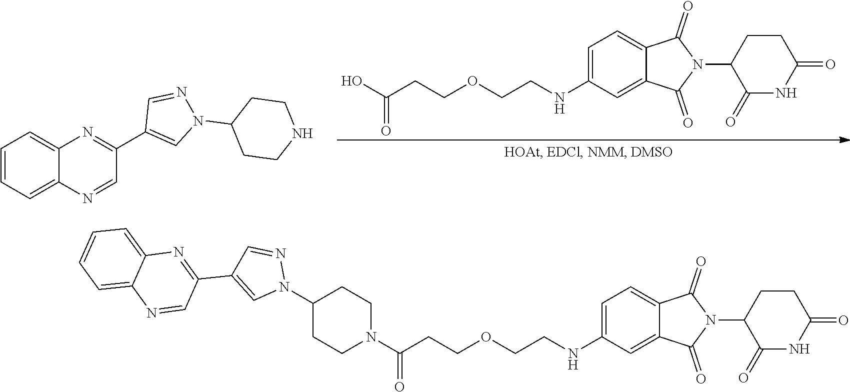

GS-324 was synthesized following the standard procedure for preparing GS-062 (7.5 mg, 40% yield) as a light-yellow solid. MS (ESI) m/z=651.3 [M+H]⁺.

### Example 283. N-(2-(2,6-Dioxopiperidin-3-yl)-1,3-dioxoisoindolin-5-yl)-6-oxo-6-(4-(4-(quinoxalin-2-yl)-1H-pyrazol-1-yl)piperidin-1-yl)hexanamide (GS-325)

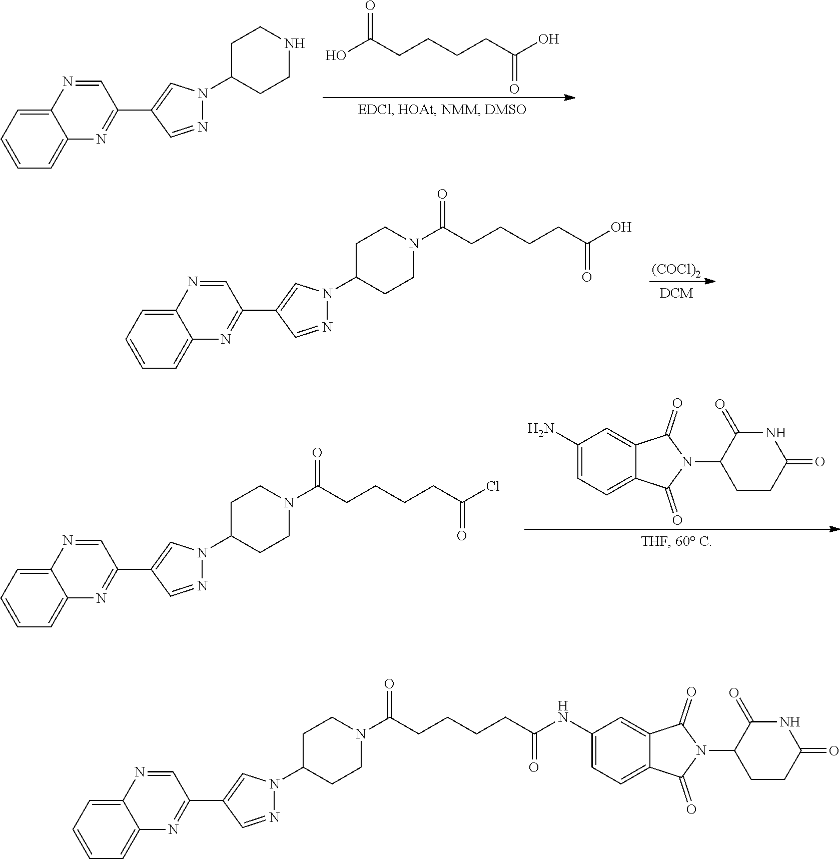

Step 1. Synthesis of 6-oxo-6-(4-(4-(quinoxalin-2-yl)-1H-pyrazol-1-yl)piperidin-1-yl)hexanoic acid

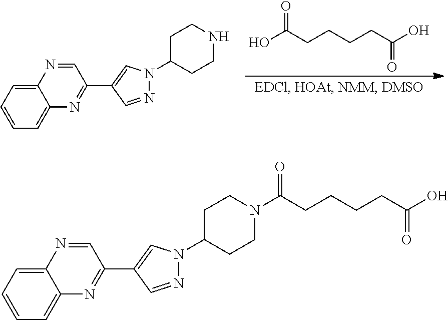 [SMILES: c1ccc2nc(-c3cnn(C4CCNCC4)c3)cnc2c1 ; O=C(O)CCCCC(=O)O ; O=C(O)CCCCC(=O)N1CCC(n2cc(-c3cnc4ccccc4n3)cn2)CC1]

A mixture of 2-(1-(piperidin-4-yl)-1H-pyrazol-4-yl)quinoxaline (50 mg, 0.18 mmol), adipic acid (263 mg, 1.8 mmol), EDCI (70 mg, 0.36 mmol), HOAt (49 mg, 0.36 mmol) and NMM (180 mg, 1.8 mmol) in DMSO (5 mL) was stirred at rt for 16 h. The reaction mixture was poured into water (30 mL) and extracted with ethyl acetate (3×15 mL). The combined organic layers were washed with saturated brine (30 mL), dried over anhydrous sodium sulfate, filtered, and concentrated under reduced pressure. The resulting residue was purified by reverse-phase chromatography to provide the desired product (61 mg, 83% yield) as a light-yellow solid. MS (ESI) m/z=406.2 [M−H]⁻.

Step 2. Synthesis of 6-oxo-6-(4-(4-(quinoxalin-2-yl)-1H-pyrazol-1-yl)piperidin-1-yl)hexanoyl chloride

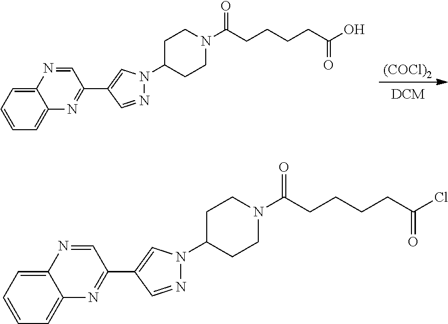

A mixture of 6-oxo-6-(4-(4-(quinoxalin-2-yl)-1H-pyrazol-1-yl)piperidin-1-yl)hexanoic acid (50 mg, 0.12 mmol) and oxalyl chloride (30 mg, 0.24 mmol) in DCM (5 mL) was stirred at rt for 1 h. The resulting mixture was concentrated to provide the crude product (46 mg, 92% yield) as a light-yellow solid which used directly in the next step without further purification.

Step 3. Synthesis of N-(2-(2,6-dioxopiperidin-3-yl)-1,3-dioxoisoindolin-5-yl)-6-oxo-6-(4-(4-(quinoxalin-2-yl)-1H-pyrazol-1-yl)piperidin-1-yl)hexanamide

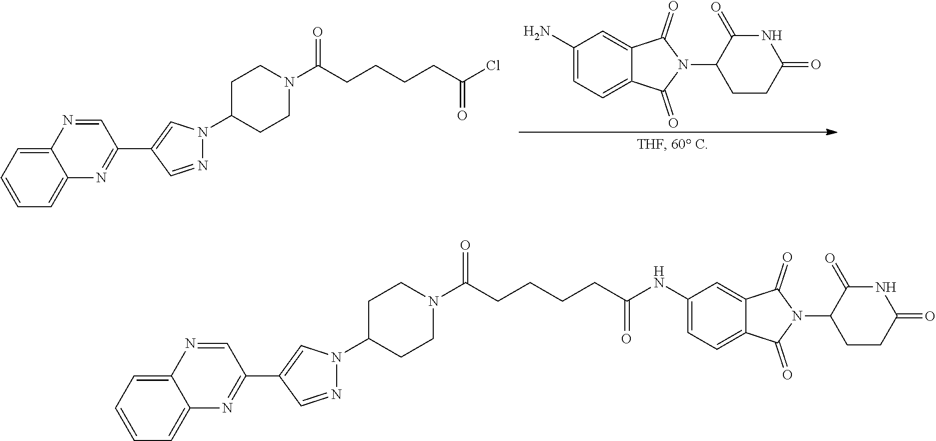 [SMILES: O=C(Cl)CCCCC(=O)N1CCC(n2cc(-c3cnc4ccccc4n3)cn2)CC1 ; Nc1ccc2c(c1)C(=O)N(C1CCC(=O)NC1=O)C2=O ; O=C1CCC(N2C(=O)c3ccc(NC(=O)CCCCC(=O)N4CCC(n5cc(-c6cnc7ccccc7n6)cn5)CC4)cc3C2=O)C(=O)N1]

A mixture of 6-oxo-6-(4-(4-(quinoxalin-2-yl)-1H-pyrazol-1-yl)piperidin-1-yl)hexanoyl chloride (10 mg, 0.024 mmol) and 5-amino-2-(2,6-dioxopiperidin-3-yl)isoindoline-1,3-dione (6 mg, 0.024 mmol) in THF (3 mL) was stirred at 60° C. for 1 h. The reaction mixture was poured into water (10 mL) and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (5.7 mg, 36% yield) as a light-yellow solid. MS (ESI) m/z=663.3 [M+H]⁺.

### Example 284. 2-(2,6-Dioxopiperidin-3-yl)-5-((6-(4-(4-(quinoxalin-2-yl)-1H-pyrazol-1-yl)piperidin-1-yl)hexyl)amino)isoindoline-1,3-dione (GS-326)

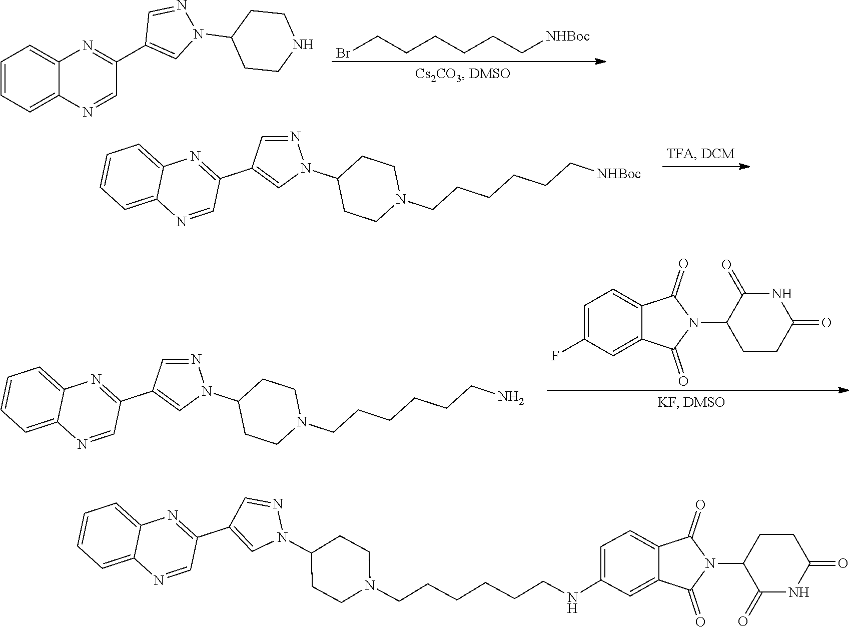 [SMILES: c1ccc2nc(-c3cnn(C4CCNCC4)c3)cnc2c1 ; CC(C)(C)OC(=O)NCCCCCCBr ; CC(C)(C)OC(=O)NCCCCCCN1CCC(n2cc(-c3cnc4ccccc4n3)cn2)CC1 ; NCCCCCCN1CCC(n2cc(-c3cnc4ccccc4n3)cn2)CC1 ; O=C1CCC(N2C(=O)c3ccc(F)cc3C2=O)C(=O)N1 ; O=C1CCC(N2C(=O)c3ccc(NCCCCCCN4CCC(n5cc(-c6cnc7ccccc7n6)cn5)CC4)cc3C2=O)C(=O)N1]

Step 1. Synthesis of tert-butyl (6-(4-(4-(quinoxalin-2-yl)-1H-pyrazol-1-yl)piperidin-1-yl)hexyl)carbamate

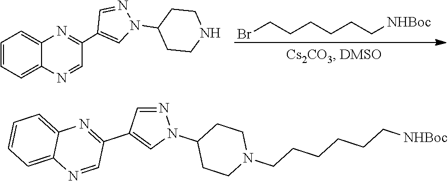 [SMILES: c1ccc2nc(-c3cnn(C4CCNCC4)c3)cnc2c1 ; CC(C)(C)OC(=O)NCCCCCCBr ; CC(C)(C)OC(=O)NCCCCCCN1CCC(n2cc(-c3cnc4ccccc4n3)cn2)CC1]

To a solution of 2-(1-(piperidin-4-yl)-1H-pyrazol-4-yl)quinoxaline (56 mg, 0.20 mmol) and tert-butyl (6-bromohexyl)carbamate (56 mg, 0.20 mmol) in DMSO (3 mL) was added Cs₂CO₃ (162 mg, 0.50 mmol) at rt. Then the reaction mixture was stirred at 80° C. for 3 h. The mixture was purified by reverse-phase chromatography to provide the desired product (50 mg, 52% yield) as a light-yellow solid. MS (ESI) m/z=479.3 [M+H]⁺.

Step 2. Synthesis of 6-(4-(4-(quinoxalin-2-yl)-1H-pyrazol-1-yl)piperidin-1-yl)hexan-1-amine

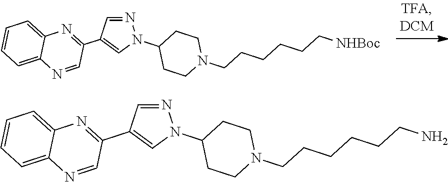

A mixture of tert-butyl (6-(4-(4-(quinoxalin-2-yl)-1H-pyrazol-1-yl)piperidin-1-yl)hexyl)carbamate (48 mg, 0.10 mmol) in TFA/DCM (1:1, 4 mL) was stirred at rt for 1 h. The resulting mixture was concentrated to provide the crude product (30 mg, 77% yield) as a light-yellow solid. This compound was used directly in the next step without further purification. MS (ESI) m/z=379.3 [M+H]⁺.

Step 3. Synthesis of 2-(2,6-dioxopiperidin-3-yl)-5-((6-(4-(4-(quinoxalin-2-yl)-1H-pyrazol-1-yl)piperidin-1-yl)hexyl)amino)isoindoline-1,3-dione

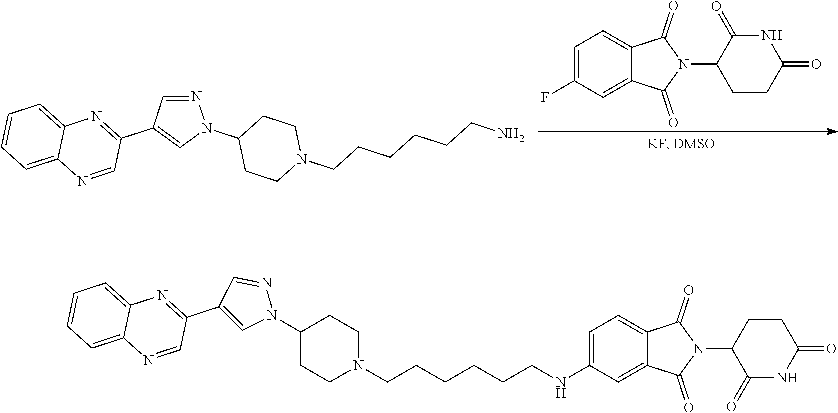 [SMILES: NCCCCCCN1CCC(n2cc(-c3cnc4ccccc4n3)cn2)CC1 ; O=C1CCC(N2C(=O)c3ccc(F)cc3C2=O)C(=O)N1 ; O=C1CCC(N2C(=O)c3ccc(NCCCCCCN4CCC(n5cc(-c6cnc7ccccc7n6)cn5)CC4)cc3C2=O)C(=O)N1]

To a solution of 6-(4-(4-(quinoxalin-2-yl)-1H-pyrazol-1-yl)piperidin-1-yl)hexan-1-amine (30 mg, 0.08 mmol) and 2-(2,6-dioxopiperidin-3-yl)-5-fluoroisoindoline-1,3-dione (22 mg, 0.08 mmol) in DMSO (2 mL) was added KF (22 mg, 0.40 mmol) at rt. Then the reaction mixture was stirred at 130° C. for 1 h. After cooling down to rt, the mixture was purified by reverse-phase chromatography to provide the desired product (15 mg, 60% yield) as a light-yellow solid. MS (ESI) m/z=635.4 [M+H]⁺.

### Example 285. N-(2-(2,6-Dioxopiperidin-3-yl)-1,3-dioxoisoindolin-5-yl)-5-oxo-5-(4-(4-(quinoxalin-2-yl)-1H-pyrazol-1-yl)piperidin-1-yl)pentanamide (GS-327)

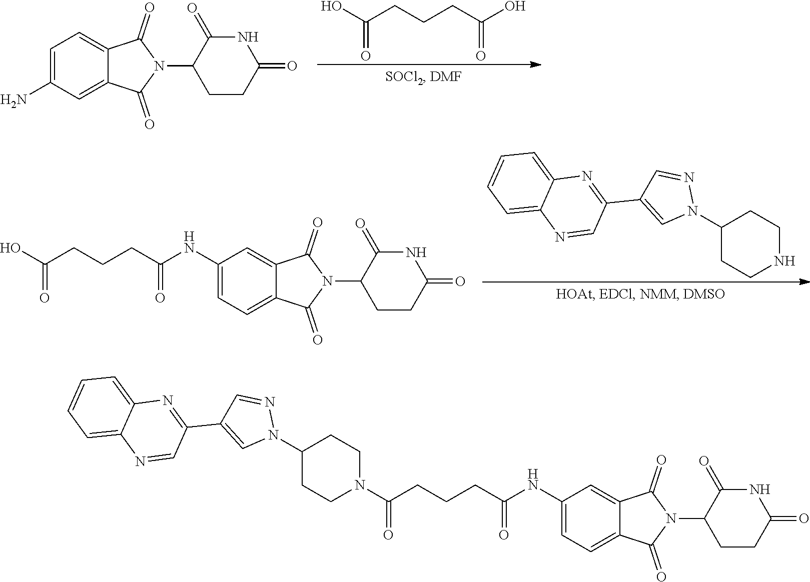 [SMILES: Nc1ccc2c(c1)C(=O)N(C1CCC(=O)NC1=O)C2=O ; O=C(O)CCCC(=O)O ; O=C(O)CCCC(=O)Nc1ccc2c(c1)C(=O)N(C1CCC(=O)NC1=O)C2=O ; c1ccc2nc(-c3cnn(C4CCNCC4)c3)cnc2c1 ; O=C1CCC(N2C(=O)c3ccc(NC(=O)CCCC(=O)N4CCC(n5cc(-c6cnc7ccccc7n6)cn5)CC4)cc3C2=O)C(=O)N1]

Step 1. Synthesis of 5-((2-(2,6-dioxopiperidin-3-yl)-1,3-dioxoisoindolin-5-yl)amino)-5-oxopentanoic acid

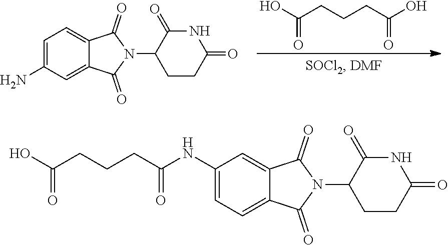 [SMILES: Nc1ccc2c(c1)C(=O)N(C1CCC(=O)NC1=O)C2=O ; O=C(O)CCCC(=O)O ; O=C(O)CCCC(=O)Nc1ccc2c(c1)C(=O)N(C1CCC(=O)NC1=O)C2=O]

A solution of glutaric acid (132 mg, 1.00 mmol) in SOCl₂ (5 mL) was stirred at reflux for 1 h. Then the mixture was evaporated under reduced pressure. The resulting residue was dissolved in DCM (5 mL) and added into a solution of 5-amino-2-(2,6-dioxopiperidin-3-yl)isoindoline-1,3-dione (81 mg, 0.30 μmol) and TEA (101 mg, 1.00 mmol) in DMF (5 mL) at 0° C. The mixture was stirred at rt for 1 h, before it was concentrated under reduced pressure. The resulting residue was purified by reverse-phase chromatography to provide the desired product (23 mg, 20% yield) as a light-yellow solid. MS (ESI) m/z=386.1 [M−H]⁻.

The remaining step followed the standard procedures for preparing GS-062 to provide the desired product (10 mg, 27% yield) as a light-yellow solid. MS (ESI) m/z=649.3 [M+H]⁺.

### Example 286. 5-(4-(4-(8-Bromoquinoxalin-2-yl)-1H-pyrazol-1-yl)piperidin-1-yl)-N-((2-(2,6-dioxopiperidin-3-yl)-1-oxoisoindolin-5-yl)methyl)-N-methyl-5-oxopentanamide (GS-058)

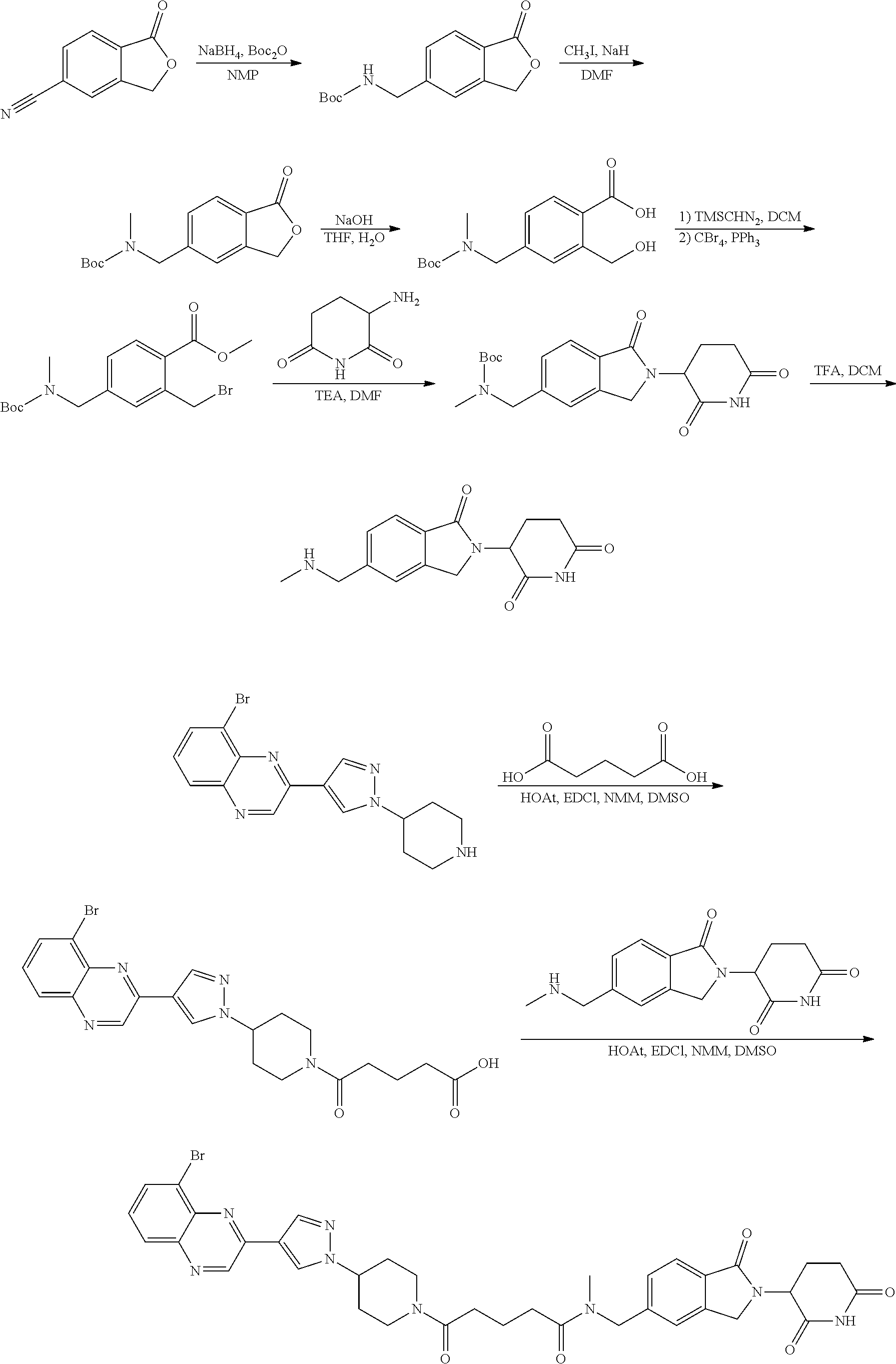

Step 1. Synthesis of tert-butyl ((1-oxo-1,3-dihydroisobenzofuran-5-yl)methyl)carbamate

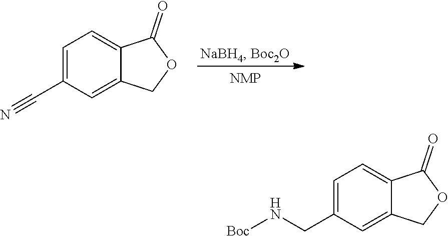 [SMILES: N#Cc1ccc2c(c1)COC2=O ; CC(C)(C)OC(=O)NCc1ccc2c(c1)COC2=O]

To a solution of tert-butyl ((1-oxo-1,3-dihydroisobenzofuran-5-yl)methyl)carbamate (1 g, 6.3 mmol) and Boc₂O (2.0 g, 9.4 mmol) in NMP (10 mL) was added NaBH₄ (1 g, 25.2 mmol) at 0° C. The reaction mixture was stirred at rt under inert atmosphere for 8 h. The mixture was poured into water (50 mL) and extracted with ethyl acetate (30 mL×3). The combined organic phase was washed with brine (20 mL), dried over anhydrous sodium sulfate, filtered, and concentrated. The resulting residue was purified by reverse-phase chromatography to provide the desired product (1.0 g, 60% yield) as a white solid. MS (ESI) m/z=264.1 [M+H]⁺.

Step 2. Synthesis of tert-butyl methyl((1-oxo-1,3-dihydroisobenzofuran-5-yl)methyl)carbamate

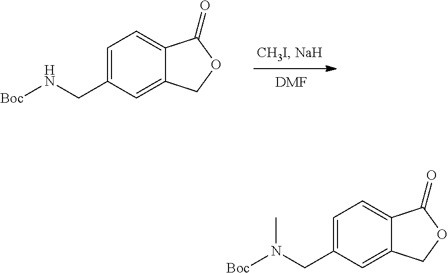

To a solution of tert-butyl ((1-oxo-1,3-dihydroisobenzofuran-5-yl)methyl)carbamate (800 mg, 3.0 mmol) in DMF (10 mL) was added NaH (243 mg, 6.1 mmol) at 0° C. The reaction mixture was stirred at 0° C. under inert atmosphere for 0.5 h. Then CH₃I (864 mg, 6.1 mmol) was added dropwise. After the solution was stirred at 0° C. for another 2 h, the reaction was quenched with aq. NH₄Cl (30 mL) slowly. The reaction mixture was extracted with ethyl acetate (30 mL×3). The organic layers were combined, washed with saturated brine (20 mL), dried over anhydrous sodium sulfate, filtered, concentrated, and the resulting residue was purified by reverse-phase chromatography to provide the desired product (500 mg, 59% yield) as a light-yellow solid. MS (ESI) m/z=278.2 [M+H]⁺.

Step 3. Synthesis of 4-(((tert-butoxycarbonyl)(methyl)amino)methyl)-2-(hydroxymethyl)benzoic acid

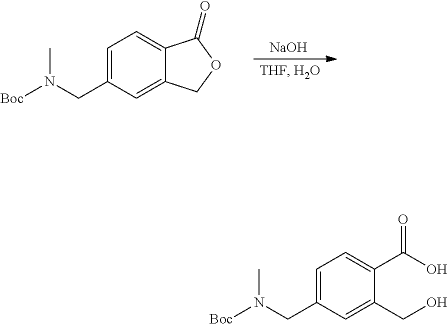

To a solution of tert-butyl ((1-oxo-1,3-dihydroisobenzofuran-5-yl)methyl)carbamate (300 mg, 1.1 mmol) in THF (10 mL) were added NaOH (173 mg, 4.3 mmol) and water (5 mL). The mixture was stirred at reflux under inert atmosphere for 1 h. The reaction was concentrated and purified by reverse-phase chromatography to provide the desired product (204 mg, 64% yield) as a light-yellow solid. MS (ESI) m/z=294.2 [M−H]⁻.

Step 4. Synthesis of methyl 2-(bromomethyl)-4-(((tert-butoxycarbonyl)(methyl)amino)methyl)benzoate

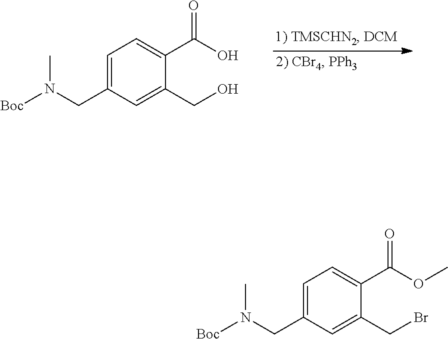

To a solution of 4-(((tert-butoxycarbonyl)(methyl)amino)methyl)-2-(hydroxymethyl)benzoic acid (50.0 mg, 0.17 mmol) in DCM (5 mL) was added TMSCHN₂ (1M, 0.34 mL) at 0° C. The mixture was stirred at 0° C. under inert atmosphere for 1 h. Then to the above mixture was added CBr₄ (84.6 mg, 0.25 mmol) and PPh₃ (67.5 mg, 0.25 mmol) at 0° C. After stirring at 0° C. for another 30 min, the solution was filtered, and concentrated. The resulting residue was directly used in next step without purification. MS (ESI) m/z=372.0 [M+H]⁺.

Step 5. Synthesis of tert-butyl ((2-(2,6-dioxopiperidin-3-yl)-1-oxoisoindolin-5-yl)methyl)(methyl)carbamate

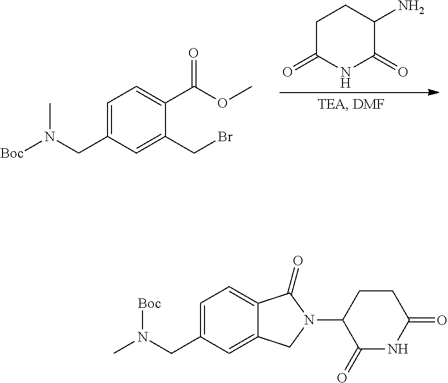

To a solution of methyl 2-(bromomethyl)-4-(((tert-butoxycarbonyl)(methyl)amino)methyl)benzoate (40.0 mg, 0.11 mmol) in DMF (2 mL) were added 3-aminopiperidine-2,6-dione hydrochloride (27.5 mg, 0.16 mmol) and TEA (33.1 mg, 0.32 mmol). Then the mixture was stirred at 80° C. under inert atmosphere overnight. The solution was concentrated and purified by reverse-phase chromatography to provide the desired product (27.8 mg, 65% yield) as a light-yellow solid. MS (ESI) m/z=388.3 [M+H]⁺.

Step 6. Synthesis of 3-(5-((methylamino)methyl)-1-oxoisoindolin-2-yl)piperidine-2,6-dione

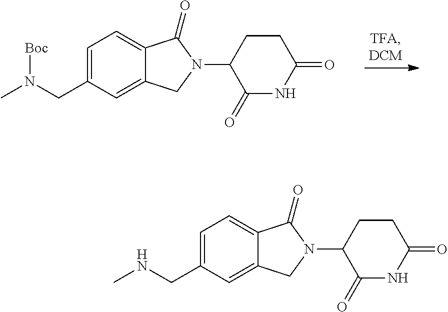 [SMILES: CN(Cc1ccc2c(c1)CN(C1CCC(=O)NC1=O)C2=O)C(=O)OC(C)(C)C ; CNCc1ccc2c(c1)CN(C1CCC(=O)NC1=O)C2=O]

A mixture of tert-butyl ((2-(2,6-dioxopiperidin-3-yl)-1-oxoisoindolin-5-yl)methyl)(methyl)carbamate (27.2 mg, 0.07 mmol) in DCM (2 mL) and TFA (1 mL) was stirred at rt for 0.5 h. Then the solution was concentrated and purified by reverse-phase chromatography to provide the desired product (15.0 mg, 75% yield) as a light-yellow solid. MS (ESI) m/z=288.3 [M+H]⁺.

Step 7. Synthesis of 5-(4-(4-(8-bromoquinoxalin-2-yl)-1H-pyrazol-1-yl)piperidin-1-yl)-5-oxopentanoic acid

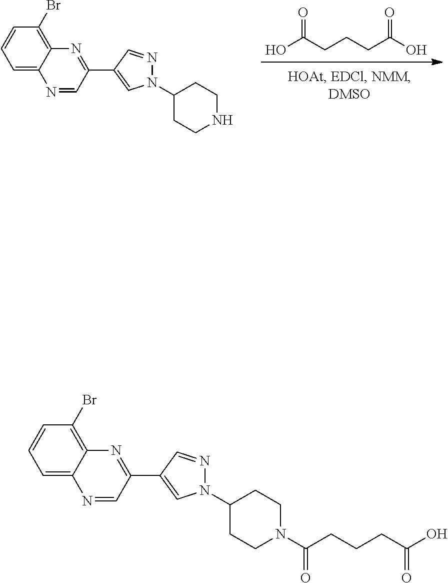 [SMILES: Brc1cccc2ncc(-c3cnn(C4CCNCC4)c3)nc12 ; O=C(O)CCCC(=O)O ; O=C(O)CCCC(=O)N1CCC(n2cc(-c3cnc4cccc(Br)c4n3)cn2)CC1]

To a solution of glutaric acid (15.4 mg, 0.11 mmol) in DMSO (1.5 mL) were added HOAt (15.5 mg, 0.11 mmol), EDCI (21.1 mg, 0.11 mmol), NMM (28.3 mg, 0.28 mmol) and 8-bromo-2-(1-(piperidin-4-yl)-1H-pyrazol-4-yl)quinoxaline (20.0 mg, 0.06 mmol) sequentially. The resulting solution was stirred at 25° C. overnight, before it was purified by reverse-phase chromatography to provide the desired product (17 mg, 31% yield) as a light-yellow solid. MS (ESI) m/z=472.2 [M+H]⁺.

Step 8. Synthesis of 5-(4-(4-(8-bromoquinoxalin-2-yl)-1H-pyrazol-1-yl)piperidin-1-yl)-N-((2-(2,6-dioxopiperidin-3-yl)-1-oxoisoindolin-5-yl)methyl)-N-methyl-5-oxopentanamide

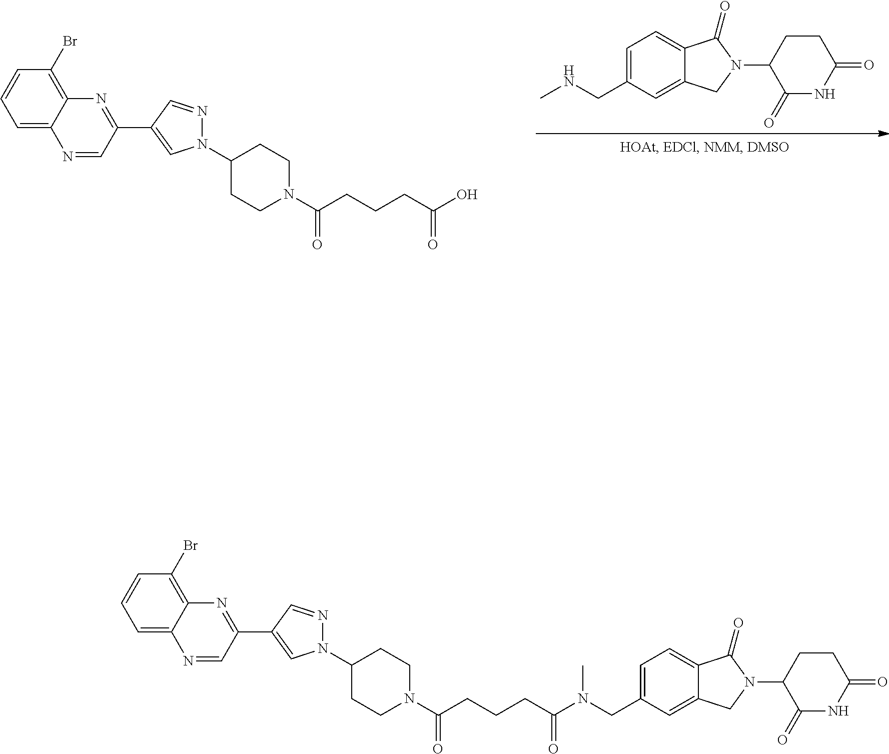 [SMILES: O=C(O)CCCC(=O)N1CCC(n2cc(-c3cnc4cccc(Br)c4n3)cn2)CC1 ; CNCc1ccc2c(c1)CN(C1CCC(=O)NC1=O)C2=O ; CN(Cc1ccc2c(c1)CN(C1CCC(=O)NC1=O)C2=O)C(=O)CCCC(=O)N1CCC(n2cc(-c3cnc4cccc(Br)c4n3)cn2)CC1]

This step followed the standard procedure for preparing GS-062 to provide the desired product (2.2 mg, 11% yield) as a light-yellow solid. MS (ESI) m/z=741.3 [M+H]⁺.

### Example 287. 3-(1-Oxo-5-((6-oxo-6-(4-phenylpiperazin-1-yl)hexyl)oxy)isoindolin-2-yl)piperidine-2,6-dione (GS-329)

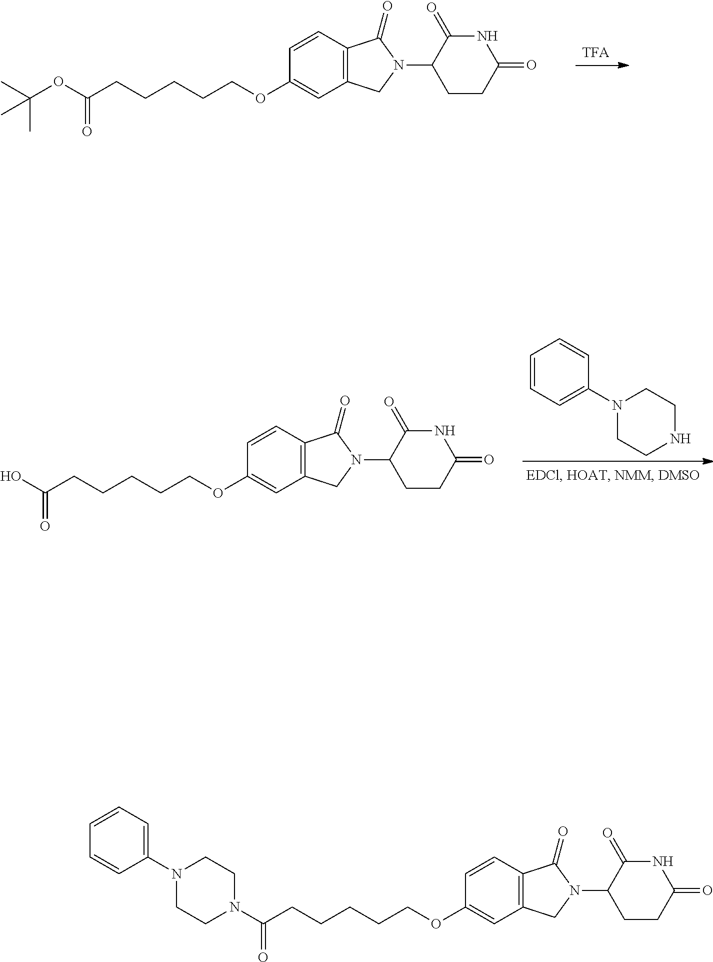 [SMILES: CC(C)(C)OC(=O)CCCCCOc1ccc2c(c1)CN(C1CCC(=O)NC1=O)C2=O ; O=C(O)CCCCCOc1ccc2c(c1)CN(C1CCC(=O)NC1=O)C2=O ; c1ccc(N2CCNCC2)cc1 ; O=C1CCC(N2Cc3cc(OCCCCCC(=O)N4CCN(c5ccccc5)CC4)ccc3C2=O)C(=O)N1]

Step 1. Synthesis of 6-((2-(2,6-dioxopiperidin-3-yl)-1-oxoisoindolin-5-yl)oxy)hexanoic acid

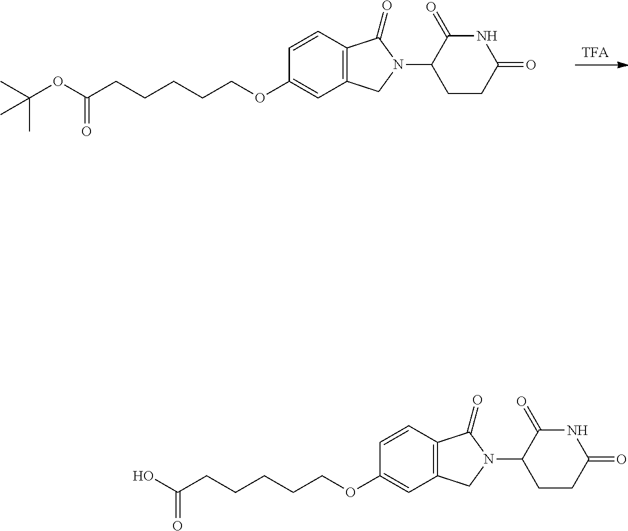 [SMILES: CC(C)(C)OC(=O)CCCCCOc1ccc2c(c1)CN(C1CCC(=O)NC1=O)C2=O ; O=C(O)CCCCCOc1ccc2c(c1)CN(C1CCC(=O)NC1=O)C2=O]

A solution of tert-butyl 6-((2-(2,6-dioxopiperidin-3-yl)-1-oxoisoindolin-5-yl)oxy)hexanoate (50 mg, 0.12 mmol) in TFA (1 mL) was stirred at rt for 1 h. Then the solvent was removed under reduced pressure. The resulting residue was used directly without further purification. MS (ESI) m/z=375.2 [M+H]⁺.

Step 2. Synthesis of 3-(1-oxo-5-((6-oxo-6-(4-phenylpiperazin-1-yl)hexyl)oxy)isoindolin-2-yl)piperidine-2,6-dion

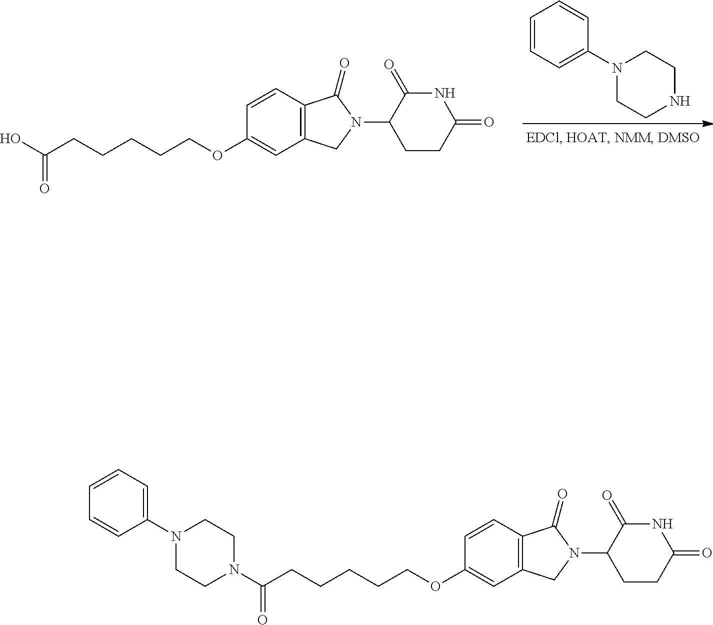 [SMILES: O=C(O)CCCCCOc1ccc2c(c1)CN(C1CCC(=O)NC1=O)C2=O ; c1ccc(N2CCNCC2)cc1 ; O=C1CCC(N2Cc3cc(OCCCCCC(=O)N4CCN(c5ccccc5)CC4)ccc3C2=O)C(=O)N1]

To a solution of 6-((2-(2,6-dioxopiperidin-3-yl)-1-oxoisoindolin-5-yl)oxy)hexanoic acid (30 mg, 0.08 mmol) in DMSO (1 mL) were added 1-phenylpiperazine (13 mg, 0.08 mmol), EDCI (23 mg, 0.12 mmol), HOAT (16 mg, 0.12 mmol) and NMM (24 mg, 0.24 mmol). The reaction mixture was stirred at 50° C. for 2 h, before it was purified by reverse phase chromatography to provide the desired product (35 mg, 68%) as a white solid. MS (ESI) m/z=519.3 [M+H]⁺.

### Example 288. N-(2-(2,6-Dioxopiperidin-3-yl)-1,3-dioxoisoindolin-5-yl)-2-(3-oxo-3-(4-(4-(quinoxalin-2-yl)-1H-pyrazol-1-yl)piperidin-1-yl)propoxy)acetamide (GS-330)

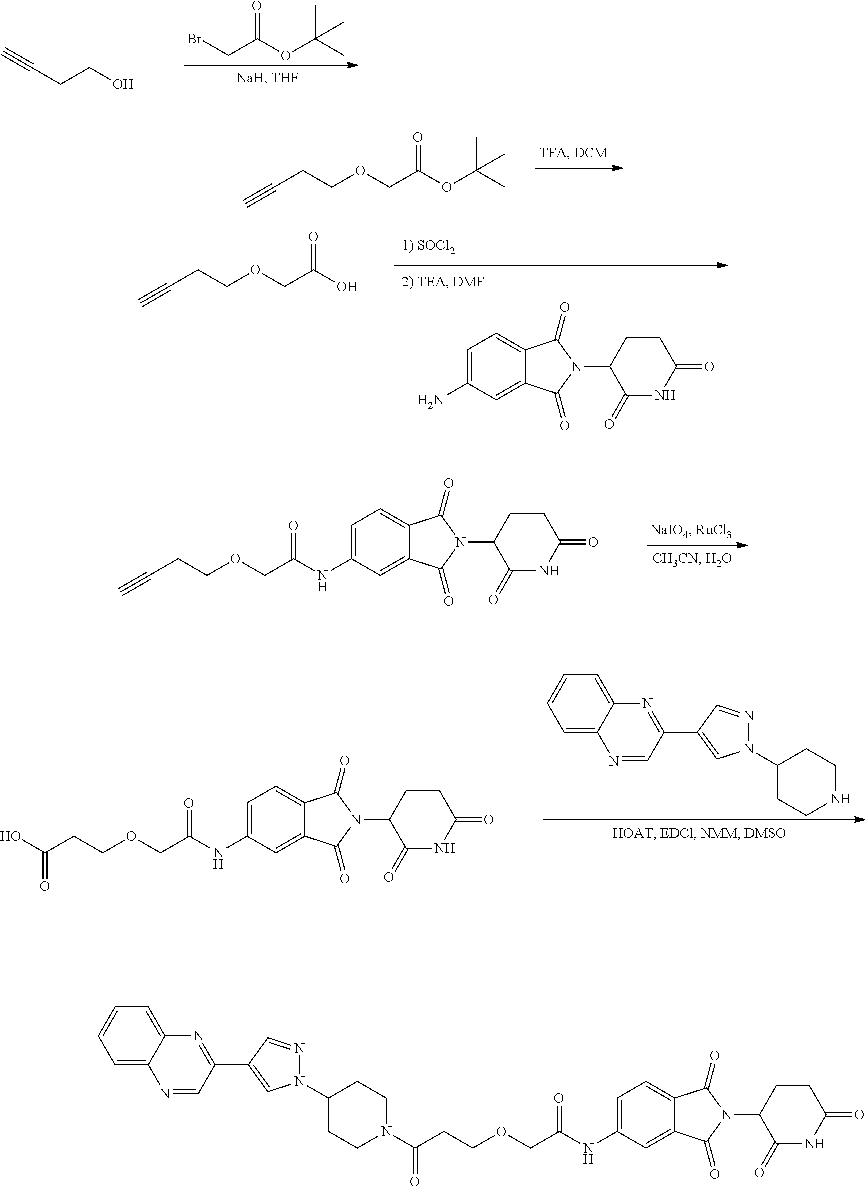

Step 1. Synthesis of tert-butyl 2-(but-3-yn-1-yloxy)acetate

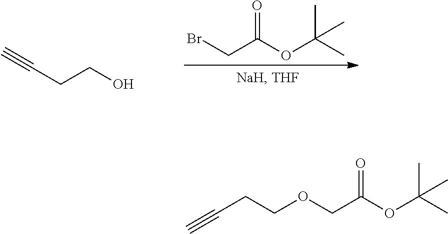

To a solution of but-3-yn-1-ol (100 mg, 1.4 mmol) in THF (10 mL) was added NaH (171 mg, 4.3 mmol) at 0° C. The solution was stirred at 0° C. under inert atmosphere for 0.5 h, before tert-butyl 2-bromoacetate (277 mg, 1.4 mmol) was added by dropwise. The resulting mixture was stirred at 0° C. for another 1 h, before it was quenched with water (5 mL) and extracted with ethyl acetate (10 mL×3). The combined organic phase was dried over anhydrous sodium sulfate, filtered, and concentrated. The resulting residue was used in the next step without further purification.

Step 2. Synthesis of 2-(but-3-yn-1-yloxy)acetic acid

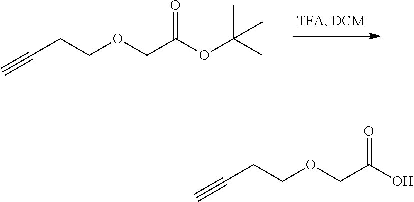

A solution of tert-butyl 2-(but-3-yn-1-yloxy)acetate (500 mg, crude) in DCM (4 mL) and TFA (2 mL) was stirred at rt for 4 h. Then the reaction mixture was concentrated to provide The resulting residue which was used in the next step without further purification.

Step 3. Synthesis of 2-(but-3-yn-1-yloxy)-N-(2-(2,6-dioxopiperidin-3-yl)-1,3-sdioxoisoindolin-5-yl)acetamide

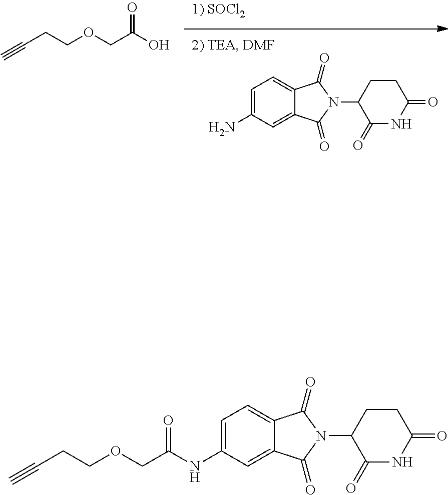

A solution of 2-(but-3-yn-1-yloxy)acetic acid (357 mg, crude) in SOCl₂ (5 mL) was stirred at reflux for 1 h. Then the mixture was concentrated. The resulting residue was dissolved in DCM (5 mL). This solution was added to a mixture of 5-amino-2-(2,6-dioxopiperidin-3-yl)isoindoline-1,3-dione (100 mg, 0.37 mmol) and TEA (74 mg, 0.74 mmol) in DMF (5 mL) at 0° C. The resulting mixture was stirred at rt for 1 h, before it was concentrated and purified by reverse-phase chromatography to provide the desired product (50 mg, 51% yield) as a light-yellow solid. MS (ESI) m/z=384.2 [M+H]⁺.

Step 4. Synthesis of 3-(2-((2-(2,6-dioxopiperidin-3-yl)-1,3-dioxoisoindolin-5-yl)amino)-2-oxoethoxy)propanoic acid

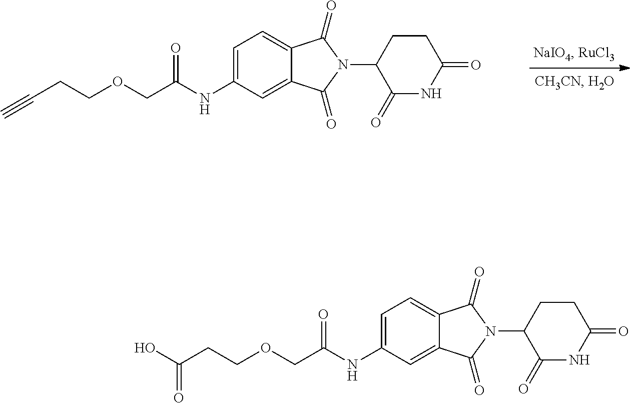 [SMILES: C#CCCOCC(=O)Nc1ccc2c(c1)C(=O)N(C1CCC(=O)NC1=O)C2=O ; O=C(O)CCOCC(=O)Nc1ccc2c(c1)C(=O)N(C1CCC(=O)NC1=O)C2=O]

To a solution of 2-(but-3-yn-1-yloxy)-N-(2-(2,6-dioxopiperidin-3-yl)-1,3-dioxoisoindolin-5-yl)acetamide (10 mg, 0.03 mmol) in CH₃CN (2 mL) and H₂O (2 mL) were added RuCl₃ (5.4 mg, 0.03 mmol) and NaIO₄ (16.8 mg, 0.78 mmol). The reaction mixture was stirred at rt for 2 h, before it was concentrated and purified by reverse-phase chromatography to provide the desired product (8.1 mg, 76% yield) as a light-yellow solid. MS (ESI) m/z=404.2 [M+H]⁺.

Step 5. Synthesis of N-(2-(2,6-dioxopiperidin-3-yl)-1,3-dioxoisoindolin-5-yl)-2-(3-oxo-3-(4-(4-(quinoxalin-2-yl)-1H-pyrazol-1-yl)piperidin-1-yl)propoxy)acetamide

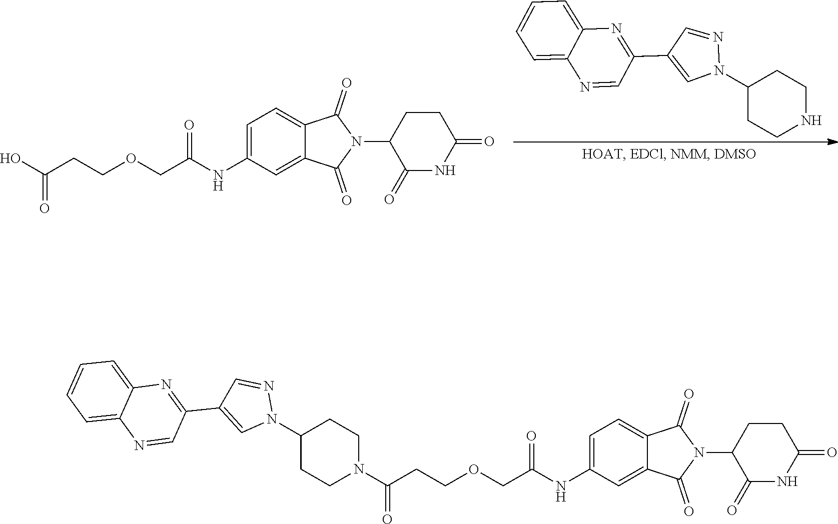 [SMILES: O=C(O)CCOCC(=O)Nc1ccc2c(c1)C(=O)N(C1CCC(=O)NC1=O)C2=O ; c1ccc2nc(-c3cnn(C4CCNCC4)c3)cnc2c1 ; O=C1CCC(N2C(=O)c3ccc(NC(=O)COCCC(=O)N4CCC(n5cc(-c6cnc7ccccc7n6)cn5)CC4)cc3C2=O)C(=O)N1]

The title compound was synthesized following the standard procedure for preparing GS-062 to provide the desired product (9.6 mg, 73% yield) as a light-yellow solid. MS (ESI) m/z=665.3 [M+H]⁺.

### Example 289. 2-(2,6-Dioxopiperidin-3-yl)-5-((6-(4-hydroxypiperidin-1-yl)hexyl)amino)isoindoline-1,3-dione (GS-331)

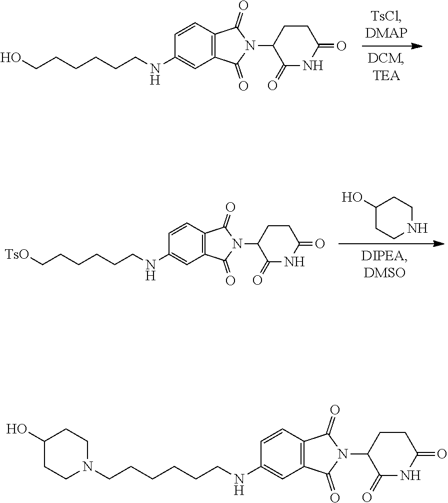 [SMILES: O=C1CCC(N2C(=O)c3ccc(NCCCCCCO)cc3C2=O)C(=O)N1 ; Cc1ccc(S(=O)(=O)OCCCCCCNc2ccc3c(c2)C(=O)N(C2CCC(=O)NC2=O)C3=O)cc1 ; OC1CCNCC1 ; O=C1CCC(N2C(=O)c3ccc(NCCCCCCN4CCC(O)CC4)cc3C2=O)C(=O)N1]

Step 1. Synthesis of 6-((2-(2,6-dioxopiperidin-3-yl)-1,3-dioxoisoindolin-5-yl)amino)hexyl 4-methylbenzenesulfonate

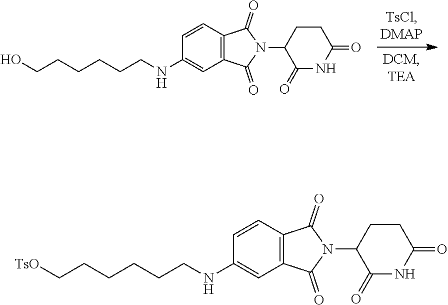 [SMILES: O=C1CCC(N2C(=O)c3ccc(NCCCCCCO)cc3C2=O)C(=O)N1 ; Cc1ccc(S(=O)(=O)OCCCCCCNc2ccc3c(c2)C(=O)N(C2CCC(=O)NC2=O)C3=O)cc1]

To a solution of 2-(2,6-dioxopiperidin-3-yl)-5-((6-hydroxyhexyl)amino)isoindoline-1,3-dione (20 mg, 0.05 mmol) in DCM (5 mL) and TEA (2 mL) were added TsCl (15 mg, 0.08 mmol) and DMAP (6 mg, 0.05 mmol) at 0° C. The reaction mixture was stirred at rt for 1 h. Then the solution was poured into water (10 mL) and extracted with DCM (3×10 mL). The combined organic layers were washed with saturated brine, dried over anhydrous sodium sulfate, filtered, and concentrated under reduced pressure. The resulting residue was purified by prep-TLC to provide the desired product (15 mg, 57% yield) as a yellow solid. MS (ESI) m/z=528.4 [M+H]⁺.

Step 2. Synthesis of 2-(2,6-dioxopiperidin-3-yl)-5-((6-(4-hydroxypiperidin-1-yl)hexyl)amino)isoindoline-1,3-dione

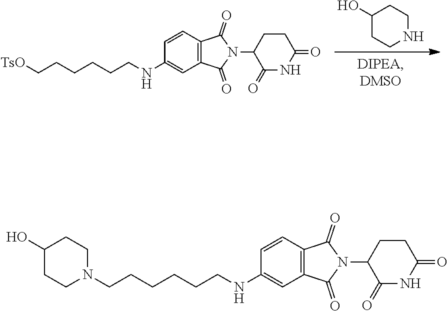 [SMILES: Cc1ccc(S(=O)(=O)OCCCCCCNc2ccc3c(c2)C(=O)N(C2CCC(=O)NC2=O)C3=O)cc1 ; OC1CCNCC1 ; O=C1CCC(N2C(=O)c3ccc(NCCCCCCN4CCC(O)CC4)cc3C2=O)C(=O)N1]

To a solution of piperidin-4-ol (2 mg, 0.02 mmol) and 6-((2-(2,6-dioxopiperidin-3-yl)-1,3-dioxoisoindolin-5-yl)amino)hexyl 4-methylbenzenesulfonate (5 mg, 0.01 mmol) in DMSO (1 mL) were added NaI (3 mg, 0.02 mmol) and DIPEA (4 mg, 0.03 mmol) at rt. The reaction mixture was stirred at 100° C. for 3 h. After cooling down to rt, the mixture was purified by reverse-phase chromatography, followed by prep-TLC to provide the desired product (2.6 mg, 58% yield) as a yellow solid. MS (ESI) m/z=457.4 [M+H]⁺.

### Example 290. N-((2-(2,6-Dioxopiperidin-3-yl)-1,3-dioxoisoindolin-5-yl)methyl)-2-(3-(4-(pyridin-2-yl)piperazin-1-yl)pyrrolidin-1-yl)acetamide (GS-332)

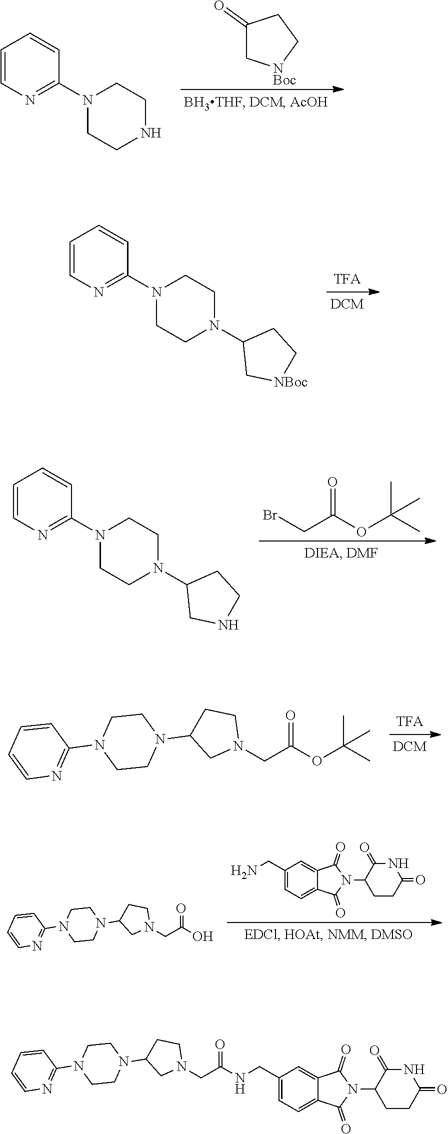

Step 1. Synthesis of tert-butyl 3-(4-(pyridin-2-yl)piperazin-1-yl)pyrrolidine-1-carboxylate

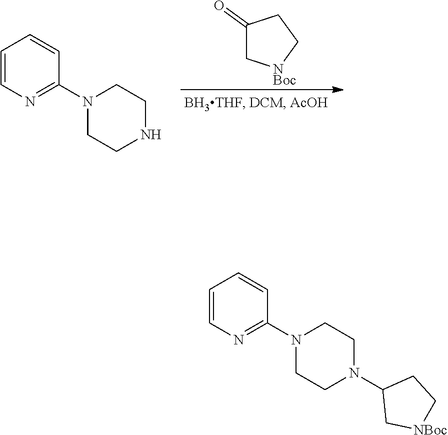 [SMILES: c1ccc(N2CCNCC2)nc1 ; CC(C)(C)OC(=O)N1CCC(=O)C1 ; CC(C)(C)OC(=O)N1CCC(N2CCN(c3ccccn3)CC2)C1]

To a solution of 1-(pyridin-2-yl)piperazine (200 mg, 1.23 mmol) and tert-butyl 3-oxopyrrolidine-1-carboxylate (453 mg, 2.46 mmol) in DCM (10 mL) and AcOH (1 mL) was added BH₃ (1M in THF, 2.5 mL, 2.46 mmol) in portions at 0° C. under N₂. The reaction mixture was stirred at rt for 2 h, before it was quenched with water slowly and extracted with DCM (3×10 mL). The combined organic layers were washed with saturated brine, dried over anhydrous sodium sulfate, filtered, and concentrated under reduced pressure. The resulting residue was purified by silica gel flash chromatography to provide the desired product (180 mg, 44% yield) as a white solid. MS (ESI) m/z=333.4 [M+H]⁺.

The remaining steps were performed according to the procedures for preparing GS-321 to provide the desired product (4.4 mg, 21% yield) as a white solid. MS (ESI) m/z=560.4 [M+H]⁺.

### Example 291. 2-(2,6-Dioxopiperidin-3-yl)-5-((6-(4-hydroxy-4-phenylpiperidin-1-yl)-6-oxohexyl)amino)isoindoline-1,3-dione (GS-333)

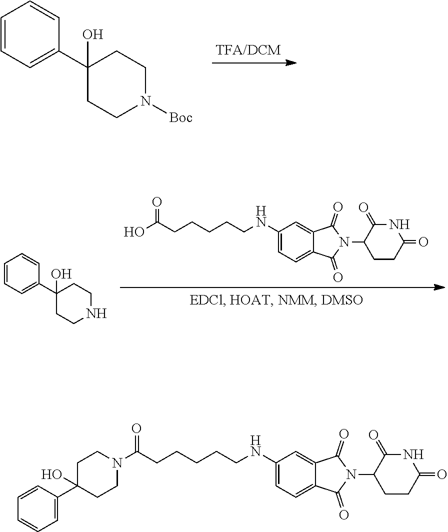 [SMILES: CC(C)(C)OC(=O)N1CCC(O)(c2ccccc2)CC1 ; OC1(c2ccccc2)CCNCC1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(O)(c5ccccc5)CC4)cc3C2=O)C(=O)N1]

Step 1. Synthesis of 4-phenylpiperidin-4-ol

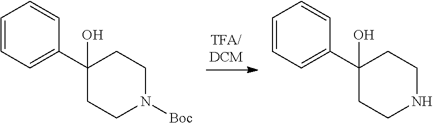 [SMILES: CC(C)(C)OC(=O)N1CCC(O)(c2ccccc2)CC1 ; OC1(c2ccccc2)CCNCC1]

To a solution of tert-butyl 4-hydroxy-4-phenylpiperidine-1-carboxylate (10 mg, 0.04 mmol) in DCM (2 mL) was added TFA (1 mL) at 0° C. After stirring at rt for 1 h, the reaction was concentrated under vacuum. The resulting residue was used in the next step directly without further purification.

Step 2. Synthesis of 2-(2,6-dioxopiperidin-3-yl)-5-((6-(4-hydroxy-4-phenylpiperidin-1-yl)-6-oxohexyl)amino)isoindoline-1,3-dione

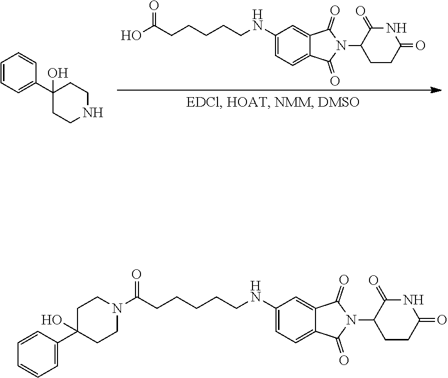 [SMILES: OC1(c2ccccc2)CCNCC1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(O)(c5ccccc5)CC4)cc3C2=O)C(=O)N1]

To a mixture of 4-phenylpiperidin-4-ol (10 mg, 0.04 mmol) in DMSO (1 mL) were added 6-((2-(2,6-dioxopiperidin-3-yl)-1,3-dioxoisoindolin-5-yl)amino)hexanoic acid (12 mg, 0.04 mmol), EDCI (11 mg, 0.06 mmol), HOAT (8 mg, 0.06 mmol) and NMM (20 mg, 0. 3 mmol). After stirring at rt for 16 h, the reaction was purified by reverse-phase chromatography, followed by prep-TLC to provide the desired product (2.5 mg, 12% yield) as a yellow solid. MS (ESI) m/z=547.3 [M+H]⁺.

### Example 292. 2-(2,6-Dioxopiperidin-3-yl)-5-((6-oxo-6-(3-phenoxyazetidin-1-yl)hexyl)amino)isoindoline-1,3-dione (GS-334)

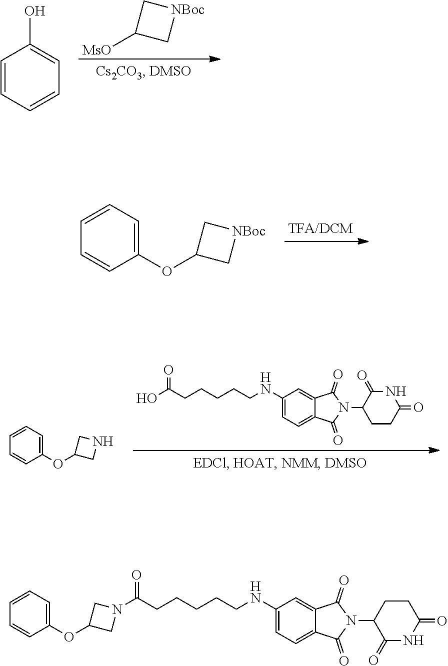 [SMILES: Oc1ccccc1 ; CC(C)(C)OC(=O)N1CC(OS(C)(=O)=O)C1 ; CC(C)(C)OC(=O)N1CC(Oc2ccccc2)C1 ; c1ccc(OC2CNC2)cc1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CC(Oc5ccccc5)C4)cc3C2=O)C(=O)N1]

Step 1. Synthesis of tert-butyl 3-phenoxyazetidine-1-carboxylate

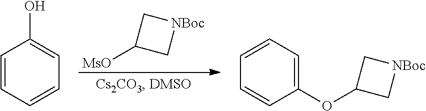 [SMILES: Oc1ccccc1 ; CC(C)(C)OC(=O)N1CC(OS(C)(=O)=O)C1 ; CC(C)(C)OC(=O)N1CC(Oc2ccccc2)C1]

To a solution of phenol (100 mg, 106 mmol) in DMF (5 mL) were added tert-butyl 3-((methylsulfonyl)oxy)azetidine-1-carboxylate (534 mg, 2.1 mmol) and Cs₂CO₃ (1.1 g, 3.18 mmol). The reaction mixture was stirred at 100° C. for 16 h. The solution was poured into water (10 mL) and extracted with ethyl acetate (3×10 mL). The combined organic layers were washed with saturated brine, dried over anhydrous sodium sulfate, filtered, and concentrated under reduced pressure. The resulting residue was purified by silica gel flash chromatography to provide the desired product (110 mg, 55% yield) as a colorless oil.

The remaining steps were performed according to the procedures for preparing GS-333 to provide the desired product (2.8 mg, 18% yield) as a yellow solid. MS (ESI) m/z=519.4 [M+H]⁺.

### Example 293. 2-(2,6-Dioxopiperidin-3-yl)-5-((6-(4-(4-hydroxycyclohexyl)piperidin-1-yl)hexyl)amino)isoindoline-1,3-dione (GS-335)

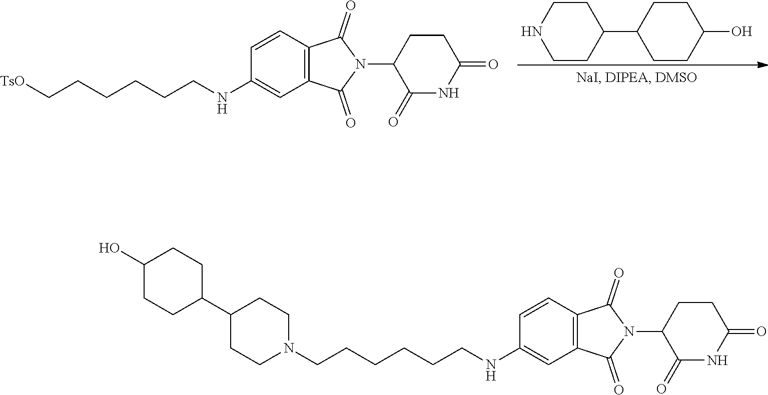 [SMILES: Cc1ccc(S(=O)(=O)OCCCCCCNc2ccc3c(c2)C(=O)N(C2CCC(=O)NC2=O)C3=O)cc1 ; OC1CCC(C2CCNCC2)CC1 ; O=C1CCC(N2C(=O)c3ccc(NCCCCCCN4CCC(C5CCC(O)CC5)CC4)cc3C2=O)C(=O)N1]

To a solution of 4-(piperidin-4-yl)cyclohexan-1-ol (6 mg, 0.02 mmol) and 6-((2-(2,6-dioxopiperidin-3-yl)-1,3-dioxoisoindolin-5-yl)amino)hexyl 4-methylbenzenesulfonate (5 mg, 0.01 mmol) in DMSO (1 mL) were added NaI (3 mg, 0.02 mmol) and DIPEA (4 mg, 0.03 mmol) at rt. The reaction mixture was stirred at 100° C. for 3 h, before it was purified by reverse-phase chromatography, followed by prep-TLC to provide the desired product (2.5 mg, 48% yield) as a yellow solid. MS (ESI) m/z=539.5 [M+H]⁺.

### Example 294. 2-(2,6-Dioxopiperidin-3-yl)-5-((6-oxo-6-(3-(phenylamino)azetidin-1-yl)hexyl)amino)isoindoline-1,3-dione (GS-336)

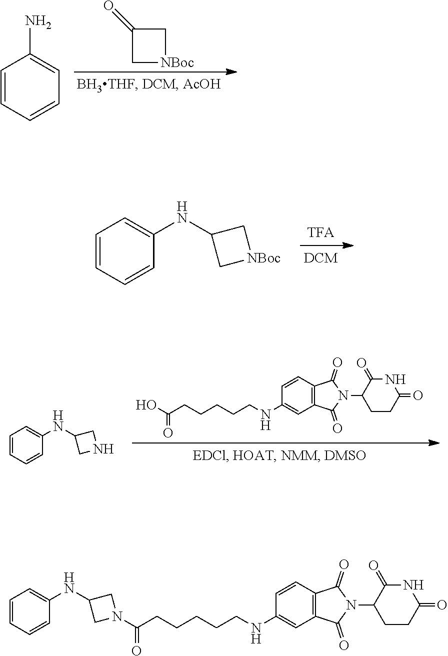 [SMILES: Nc1ccccc1 ; CC(C)(C)OC(=O)N1CC(=O)C1 ; CC(C)(C)OC(=O)N1CC(Nc2ccccc2)C1 ; c1ccc(NC2CNC2)cc1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CC(Nc5ccccc5)C4)cc3C2=O)C(=O)N1]

Step 1. Synthesis of tert-butyl 3-(phenylamino)azetidine-1-carboxylate

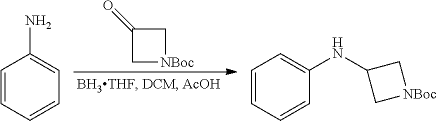 [SMILES: Nc1ccccc1 ; CC(C)(C)OC(=O)N1CC(=O)C1 ; CC(C)(C)OC(=O)N1CC(Nc2ccccc2)C1]

To a solution of aniline (300 mg, 3.2 mmol) in DCM (15 mL) were added tert-butyl 3-oxoazetidine-1-carboxylate (547 mg, 3.2 mmol), AcOH (2 drops) and BH₃·THF (1M, 3.2 mL, 3.2 mmol) at 0° C. The reaction mixture was stirred at 0° C. for 2 h, before it was concentrated and purified by silica gel flash chromatography (petroleum ether/ethyl acetate=5:1) to provide the desired product (460 mg, 57%) as a white solid. MS (ESI) m/z=249.2 [M+H]⁺.

The remaining steps were performed according to the procedures for preparing GS-333 to provide the desired product (4 mg, 57% yield) as a yellow solid. MS (ESI) m/z=518.4 [M+H]⁺.

### Example 295. 2-(2,6-Dioxopiperidin-3-yl)-5-((6-(3-(methyl(phenyl)amino)azetidin-1-yl)-6-oxohexyl)amino)isoindoline-1,3-dione (GS-337)

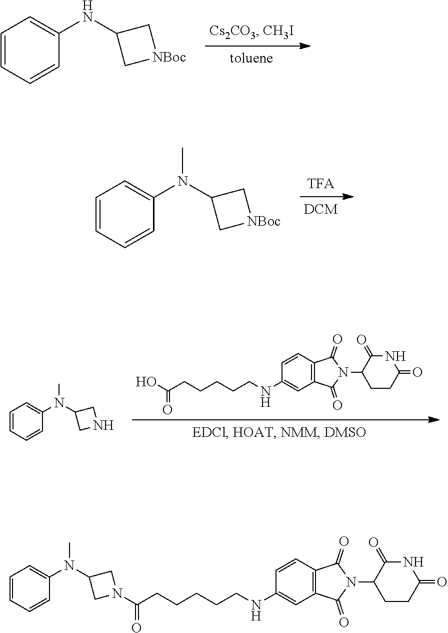 [SMILES: CC(C)(C)OC(=O)N1CC(Nc2ccccc2)C1 ; CN(c1ccccc1)C1CN(C(=O)OC(C)(C)C)C1 ; CN(c1ccccc1)C1CNC1 ; O=C(O)CCCCCNc1ccc2c(c1)C(=O)N(C1CCC(=O)NC1=O)C2=O ; CN(c1ccccc1)C1CN(C(=O)CCCCCNc2ccc3c(c2)C(=O)N(C2CCC(=O)NC2=O)C3=O)C1]

Step 1. Synthesis of tert-butyl 3-(methyl(phenyl)amino)azetidine-1-carboxylate

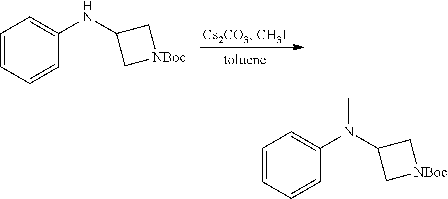 [SMILES: CC(C)(C)OC(=O)N1CC(Nc2ccccc2)C1 ; CN(c1ccccc1)C1CN(C(=O)OC(C)(C)C)C1]

To a solution of tert-butyl 3-(phenylamino)azetidine-1-carboxylate (100 mg, 0.4 mmol) in toluene (5 mL) were added Cs₂CO₃ (5 mg, 0.013 mmol) and CH₃I (284 mg, 2 mmol). The reaction mixture was stirred at 100° C. for 16 h, before it was concentrated and purified by silica gel flash chromatography (petroleum/ethyl acetate=5:1) to provide the desired prodcut (12 mg, 12%) as a yellow oil. MS (ESI) m/z=263.2 [M+H]⁺.

The remaining steps were performed according to the procedures for preparing GS-333 to provide the desired product (2 mg, 30%) as a yellow solid. MS (ESI) m/z=532.3 [M+H]⁺.

### Example 296. N-((2-(2,6-Dioxopiperidin-3-yl)-1,3-dioxoisoindolin-5-yl)methyl)-2-(3-(4-(pyridin-2-yl)piperazin-1-yl)azetidin-1-yl)acetamide (GS-338)

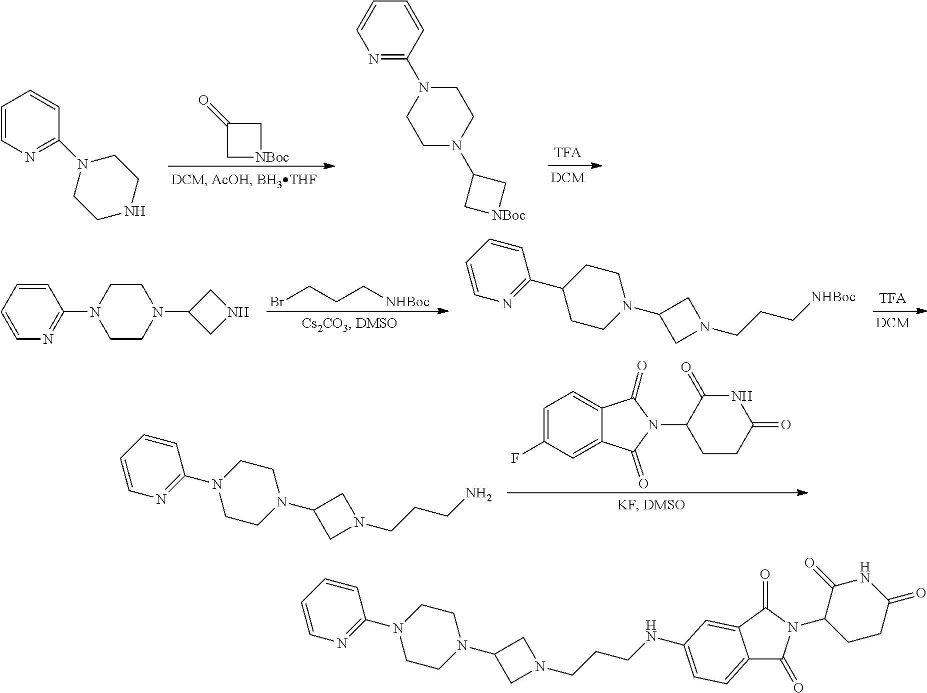

Step 1. Synthesis of tert-butyl 3-(4-(pyridin-2-yl)piperazin-1-yl)azetidine-1-carboxylate

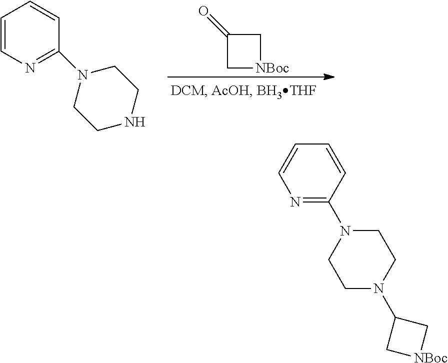 [SMILES: c1ccc(N2CCNCC2)nc1 ; CC(C)(C)OC(=O)N1CC(=O)C1 ; CC(C)(C)OC(=O)N1CC(N2CCN(c3ccccn3)CC2)C1]

A mixture of 1-(pyridin-2-yl)piperazine (64 mg, 0.40 mmol), tert-butyl 3-oxoazetidine-1-carboxylate (68 mg, 0.40 mmol), NaBH₃CN (104 mg, 1.60 mmol) and AcOH (24 mg, 0.40 mmol) in MeOH (4 mL) was stirred at 40° C. for 2 h. The mixture was poured into water (20 mL) and extracted with ethyl acetate (3×20 mL). The combined organic layers were washed with saturated brine (20 mL), dried over anhydrous sodium sulfate, filtered, and concentrated under reduced pressure. The resulting residue was purified by reverse-phase chromatography to provide the desired product (60 mg, 49% yield) as a light-yellow solid. MS (ESI) m/z=319.2 [M+H]⁺.

The remaining steps were performed according to the standard procedures for preparing GS-326 to provide the desired product (8.2 mg, 45% yield) as a light-yellow solid. MS (ESI) m/z=532.3 [M+H]⁺.

### Example 297. 2-(2,6-Dioxopiperidin-3-yl)-5-((6-(4-(quinoxalin-2-yl)-1H-pyrazol-1-yl)hexyl)amino)isoindoline-1,3-dione (GS-339)

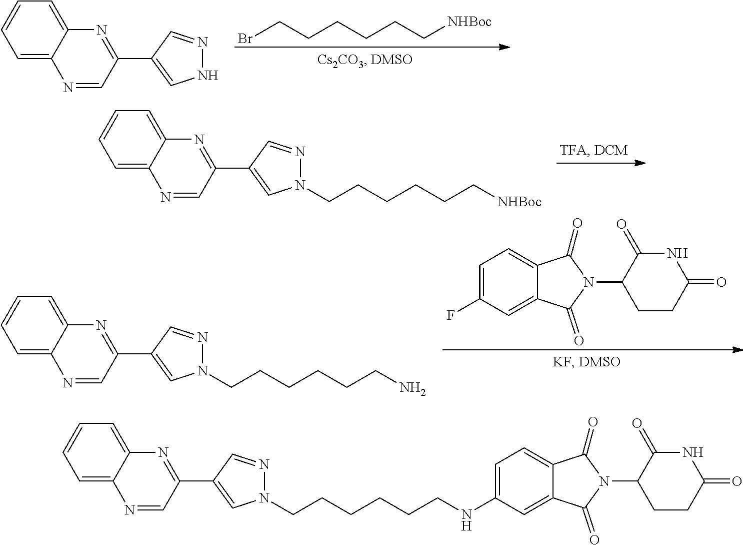

GS-339 was synthesized following the standard procedures for preparing GS-326 (9.1 mg, 51% yield) as a light-yellow solid. MS (ESI) m/z=552.3 [M+H]⁺.

### Example 298. N-((2-(2,6-Dioxopiperidin-3-yl)-1,3-dioxoisoindolin-5-yl)methyl)-2,2-difluoro-2-(3-(4-(quinoxalin-2-yl)-1H-pyrazol-1-yl)phenyl)acetamide (GS-340)

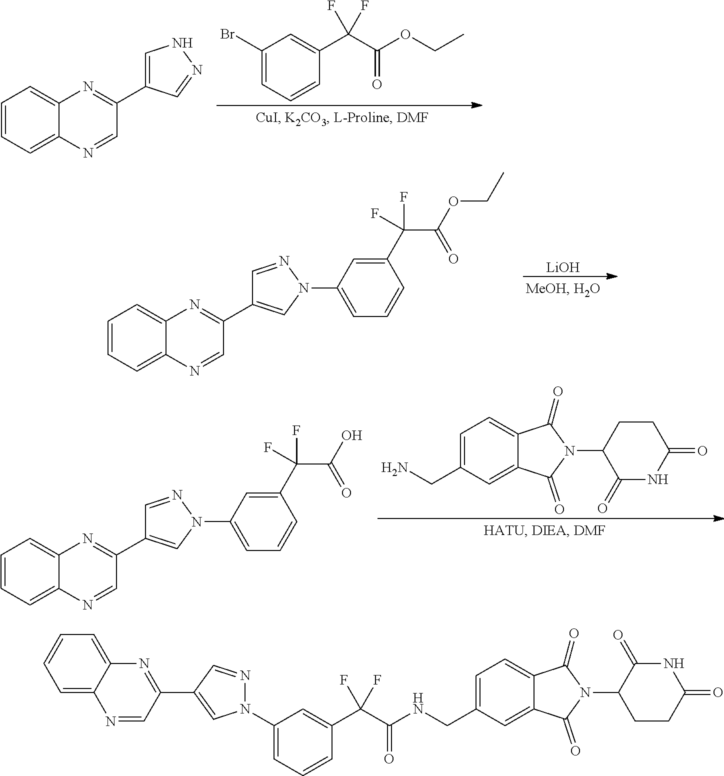

Step 1. Synthesis of ethyl 2,2-difluoro-2-(3-(4-(quinoxalin-2-yl)-1H-pyrazol-1-yl)phenyl)acetate

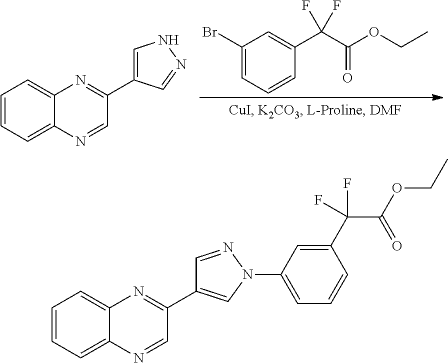 [SMILES: c1ccc2nc(-c3cn[nH]c3)cnc2c1 ; CCOC(=O)C(F)(F)c1cccc(Br)c1 ; CCOC(=O)C(F)(F)c1cccc(-n2cc(-c3cnc4ccccc4n3)cn2)c1]

To a solution of 2-(1H-pyrazol-4-yl)quinoxaline 100 mg, 0.51 mmo and ethyl 2-(3-bromophenyl)-2,2-difluoroacetate (170 mg, 0.61 mmol) in DMF (4 mL) were added K₂CO₃ (140 mg, 1.02 mmol), L-proline (18 mg, 0.15 mmol) and CuI (10 mg, 0.05 mmol) under N₂. The reaction mixture was stirred at 90° C. for 3 h, before it was concentrated and purified by reverse-phase chromatography to provide the desired product (40 mg, 20% yield) as a brown solid. MS (ESI) m/z=395.3 [M+H]⁺.

Step 2. Synthesis of 2,2-difluoro-2-(3-(4-(quinoxalin-2-yl)-1H-pyrazol-1-yl)phenyl)acetic acid

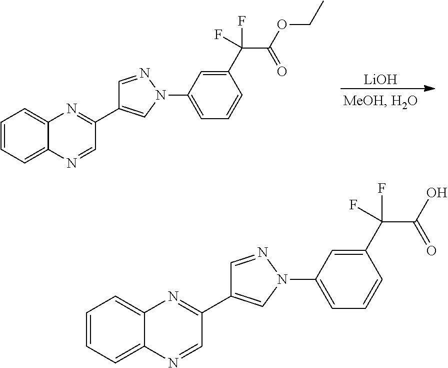 [SMILES: CCOC(=O)C(F)(F)c1cccc(-n2cc(-c3cnc4ccccc4n3)cn2)c1 ; "LiOH" ; O=C(O)C(F)(F)c1cccc(-n2cc(-c3cnc4ccccc4n3)cn2)c1]

To a solution of ethyl 2,2-difluoro-2-(3-(4-(quinoxalin-2-yl)-1H-pyrazol-1-yl)phenyl)acetate (40 mg, 0.10 mmol) in MeOH (4 mL) and H₂O (1 mL) was added LiOH·H₂O (42 mg, 1.0 mmol). After stirring at 50° C. for 4 h, the reaction mixture was concentrated. HCl (1M) was added to adjust pH to 4-5. The precipitate was collected and dried to provide the crude product (40 mg) as a brown solid. This compound was used in the next step directly without further purification. MS (ESI) m/z=367.1 [M+H]⁺.

Step 3. Synthesis of N-((2-(2,6-dioxopiperidin-3-yl)-1,3-dioxoisoindolin-5-yl)methyl)-2,2-difluoro-2-(3-(4-(quinoxalin-2-yl)-1H-pyrazol-1-yl)phenyl)acetamide

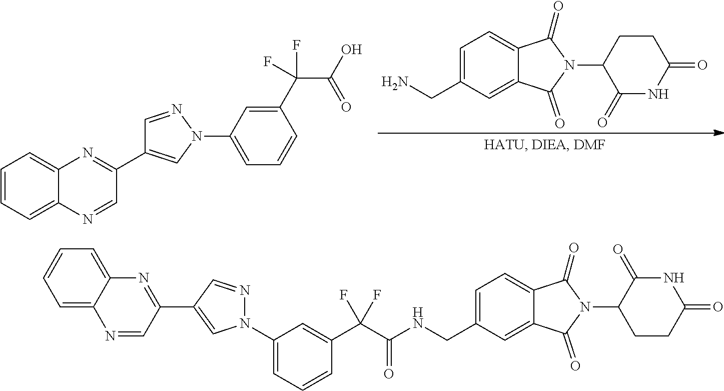

To a solution of 2,2-difluoro-2-(3-(4-(quinoxalin-2-yl)-1H-pyrazol-1-yl)phenyl)acetic acid (13 mg, 0.035 mmol) in DMF (1 mL) were added 5-(aminomethyl)-2-(2,6-dioxopiperidin-3-yl)isoindoline-1,3-dione (13 mg, 0.046 mmol), HATU (27 mg, 0.07 mmol) and DIEA (23 mg, 0.18 mmol). The reaction mixture was stirred at rt for 2 h, before it was quenched with water (5 mL) and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (5 mg, 22% yield) as a light-yellow solid. MS (ESI) m/z=636.2 [M+H]⁺.

### Example 299. 5-((6-(4-(1H-Pyrazol-1-yl)phenyl)-6-oxohexyl)amino)-2-(2,6-dioxopiperidin-3-yl)isoindoline-1,3-dione (GS-061)

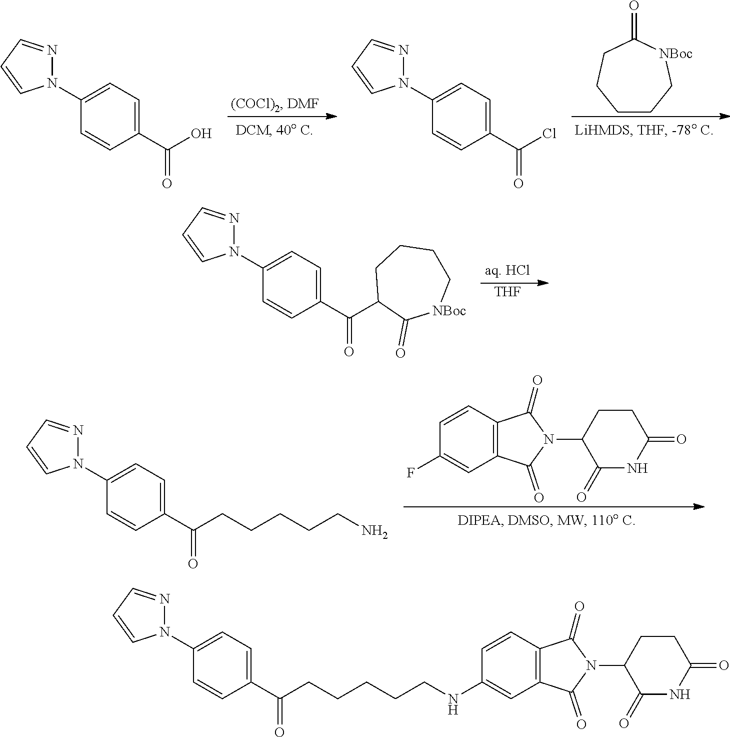

Step 1. Synthesis of 4-(1H-pyrazol-1-yl)benzoyl chloride

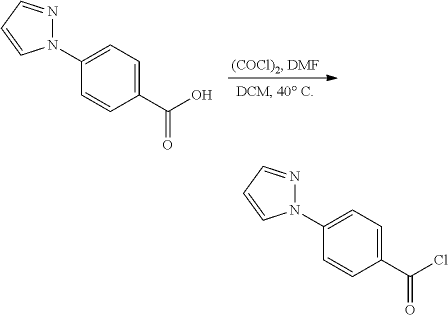 [SMILES: O=C(O)c1ccc(-n2cccn2)cc1 ; O=C(Cl)c1ccc(-n2cccn2)cc1]

To a solution of 4-(1H-pyrazol-1-yl)benzoic acid (80 mg, 0.43 mmol) in DCM (5 mL) were added oxalyl chloride (108 mg, 0.86 mmol) and DMF (1 drop) at rt. The reaction mixture was stirred at 40° C. for 1 h, before it was concentrated to provide the crude product (82 mg, 92% yield) as a light-yellow solid, which was used directly in the next step without further purification.

Step 2. Synthesis of tert-butyl 3-(4-(1H-pyrazol-1-yl)benzoyl)-2-oxoazepane-1-carboxylate

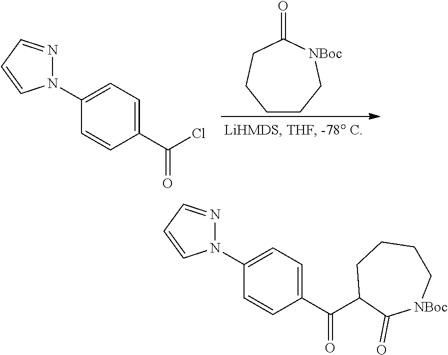

To a solution of tert-butyl 2-oxoazepane-1-carboxylate (92 mg, 0.43 mmol) in THF (3 mL) was added LiHMDS (1.0 M in THF, 0.52 mL) at −78° C. After stirring at −78° C. for 1 h, a solution of 4-(1H-pyrazol-1-yl)benzoyl chloride (82 mg, 0.40 mmol) in THF (2 mL) was added dropwise at −78° C. The reaction was stirred at −78° C. for another 1 h, before it was quenched with aq. NH₄Cl and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (95 mg, 62% yield) as a light-yellow solid. MS (ESI) m/z=384.2 [M+H]⁺.

Step 3. Synthesis of 1-(4-(1H-pyrazol-1-yl)phenyl)-6-aminohexan-1-one

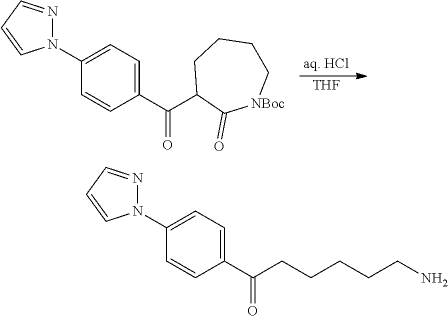

To a solution of tert-butyl 3-(4-(1H-pyrazol-1-yl)benzoyl)-2-oxoazepane-1-carboxylate (50 mg, 0.13 mmol) in THF (2 mL) was added aq. HCl (6.0 M, 3 mL) at rt. After the reaction was stirred at 60° C. for 1 h, it was concentrated under reduced pressure. The resulting residue was purified by reverse-phase chromatography to provide the desired product (22 mg, 68% yield) as a light-yellow solid. MS (ESI) m/z=258.2 [M+H]⁺.

Step 4. Synthesis of 5-((6-(4-(1H-pyrazol-1-yl)phenyl)-6-oxohexyl)amino)-2-(2,6-dioxopiperidin-3-yl)isoindoline-1,3-dione

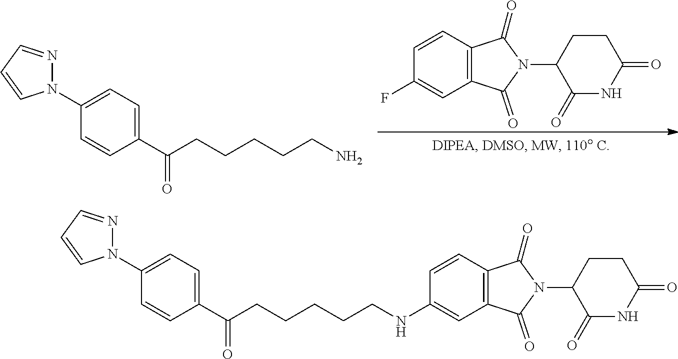 [SMILES: NCCCCCC(=O)c1ccc(-n2cccn2)cc1 ; O=C1CCC(N2C(=O)c3ccc(F)cc3C2=O)C(=O)N1 ; O=C1CCC(N2C(=O)c3ccc(NCCCCCC(=O)c4ccc(-n5cccn5)cc4)cc3C2=O)C(=O)N1]

A mixture of 1-(4-(1H-pyrazol-1-yl)phenyl)-6-aminohexan-1-one (10 mg, 0.04 mmol), 2-(2,6-dioxopiperidin-3-yl)-5-fluoroisoindoline-1,3-dione (11 mg, 0.04 mmol) and DIPEA (15 mg, 0.12 mmol) in DMSO (1.5 mL) was stirred at 110° C. for 1 h under microwave irradiation. The reaction mixture was poured into water (10 mL) and extracted with ethyl acetate (3×10 mL). The combined organic layers were washed with saturated brine (10 mL), dried over anhydrous sodium sulfate, filtered, and concentrated under reduced pressure. The resulting residue was purified by prep-TLC to provide the desired product (9.4 mg, 47% yield) as a light-yellow solid. MS (ESI) m/z=514.2 [M+H]⁺.

### Example 300. N-(2-(2,6-Dioxopiperidin-3-yl)-1,3-dioxoisoindolin-5-yl)-2-(2-oxo-2-(4-(4-(quinoxalin-2-yl)-1H-pyrazol-1-yl)piperidin-1-yl)ethoxy)acetamide (GS-342)

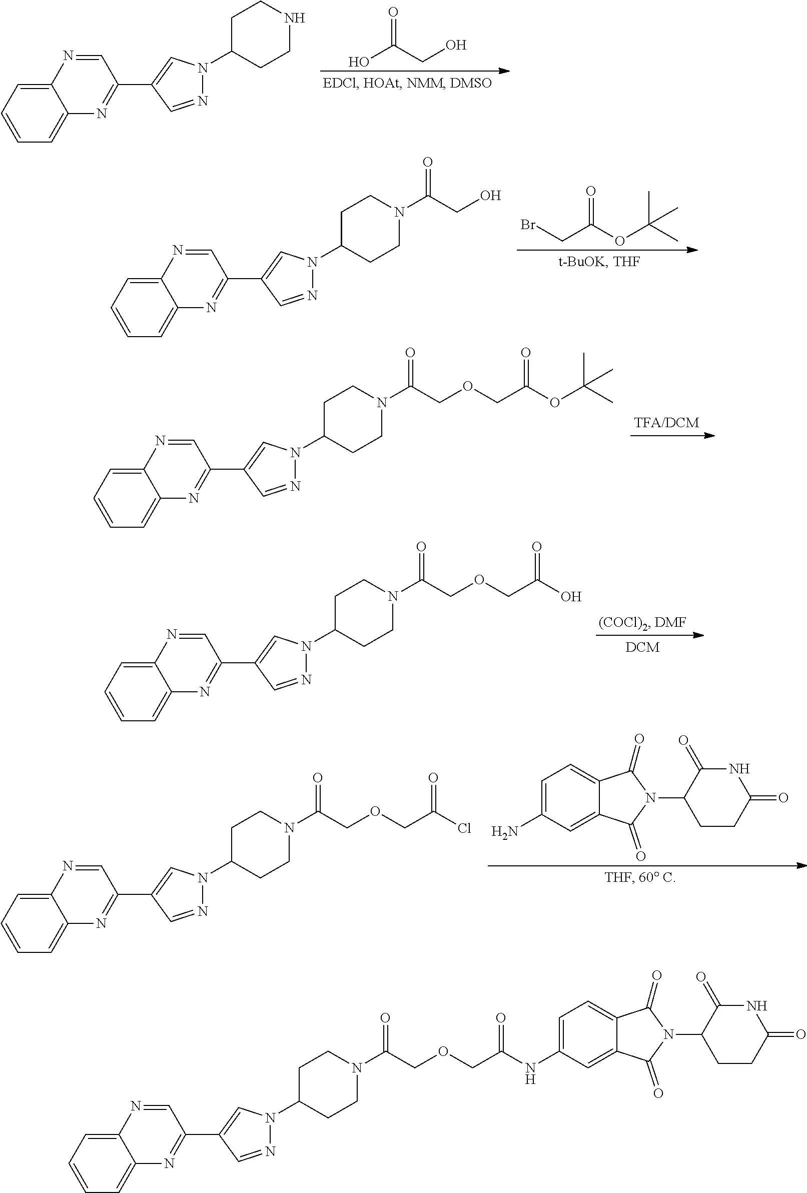

Step 1. Synthesis of 2-hydroxy-1-(4-(4-(quinoxalin-2-yl)-1H-pyrazol-1-yl)piperidin-1-yl)ethan-1-one

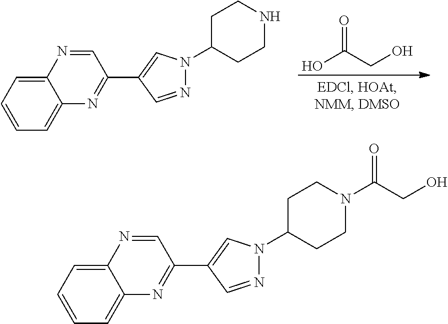 [SMILES: c1ccc2nc(-c3cnn(C4CCNCC4)c3)cnc2c1 ; O=C(O)CO ; O=C(CO)N1CCC(n2cc(-c3cnc4ccccc4n3)cn2)CC1]

A mixture of 2-(1-(piperidin-4-yl)-1H-pyrazol-4-yl)quinoxaline (100 mg, 0.36 mmol), 2-hydroxyacetic acid (27 mg, 0.36 mmol), EDCI (104 mg, 0.54 mmol), HOAt (73 mg, 0.54 mmol) and NMM (363 mg, 3.6 mmol) in DMSO (5 mL) was stirred at rt for 16 h. The reaction mixture was poured into water (20 mL) and extracted with ethyl acetate (3×20 mL). The combined organic layers were washed with saturated brine (20 mL), dried over anhydrous sodium sulfate, filtered, and concentrated under reduced pressure. The resulting residue was purified by reverse-phase chromatography to provide the desired product (107 mg, 88% yield) as a light-yellow solid. MS (ESI) m/z=338.2 [M+H]⁺.

Step 2. Synthesis of tert-butyl 2-(2-oxo-2-(4-(4-(quinoxalin-2-yl)-1H-pyrazol-1-yl)piperidin-1-yl)ethoxy)acetate

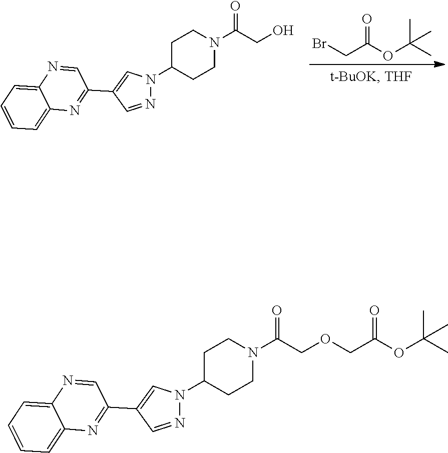

To a solution of 2-hydroxy-1-(4-(4-(quinoxalin-2-yl)-1H-pyrazol-1-yl)piperidin-1-yl)ethan-1-one (60 mg, 0.18 mmol) in THF (3 mL) was added t-BuOK (40 mg, 0.36 mL) at 0° C. After the reaction was stirred at 0° C. for 1 h, a solution of tert-butyl 2-bromoacetate (42 mg, 0.22 mmol) in THF (1 mL) was added dropwise at 0° C. The reaction was stirred at rt for 1 h, before it was poured into water (10 mL) and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (37 mg, 46% yield) as a light-yellow solid. MS (ESI) m/z=452.2 [M+H]⁺.

Step 3. Synthesis of 2-(2-oxo-2-(4-(4-(quinoxalin-2-yl)-1H-pyrazol-1-yl)piperidin-1-yl)ethoxy)acetic acid

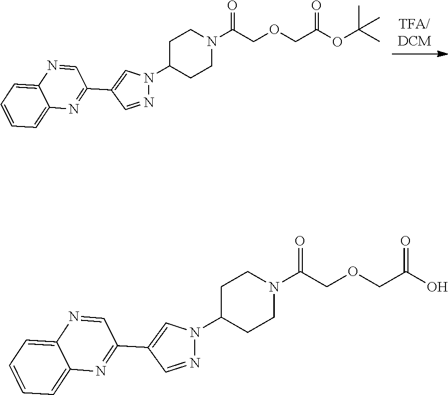 [SMILES: CC(C)(C)OC(=O)COCC(=O)N1CCC(n2cc(-c3cnc4ccccc4n3)cn2)CC1 ; O=C(O)COCC(=O)N1CCC(n2cc(-c3cnc4ccccc4n3)cn2)CC1]

A mixture of tert-butyl 2-(2-oxo-2-(4-(4-(quinoxalin-2-yl)-1H-pyrazol-1-yl) piperidin-1-yl)ethoxy) acetate (37 mg, 0.08 mmol) in TFA/DCM (1:1, 3 mL) was stirred at rt for 1 h. The resulting mixture was concentrated to provide the crude product (30 mg, 92% yield) as a light-yellow solid. This compound was used directly in the next step without further purification. MS (ESI) m/z=394.2 [M−H]⁻.

Step 4. Synthesis of 2-(2-oxo-2-(4-(4-(quinoxalin-2-yl)-1H-pyrazol-1-yl)piperidin-1-yl)ethoxy)acetyl chloride

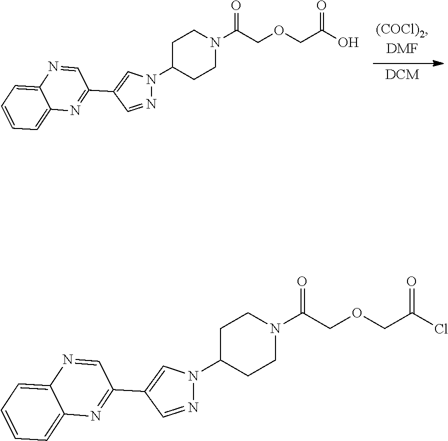 [SMILES: O=C(O)COCC(=O)N1CCC(n2cc(-c3cnc4ccccc4n3)cn2)CC1 ; O=C(Cl)COCC(=O)N1CCC(n2cc(-c3cnc4ccccc4n3)cn2)CC1]

The reaction mixture was stirred at 40° C. for 1 h, before it was concentrated to provide the crude product (31 mg, 93% yield) as a light-yellow solid which was used directly in the next step without further purification.

Step 5. Synthesis of N-(2-(2,6-dioxopiperidin-3-yl)-1,3-dioxoisoindolin-5-yl)-2-(2-oxo-2-(4-(4-(quinoxalin-2-yl)-1H-pyrazol-1-yl)piperidin-1-yl)ethoxy)acetamide

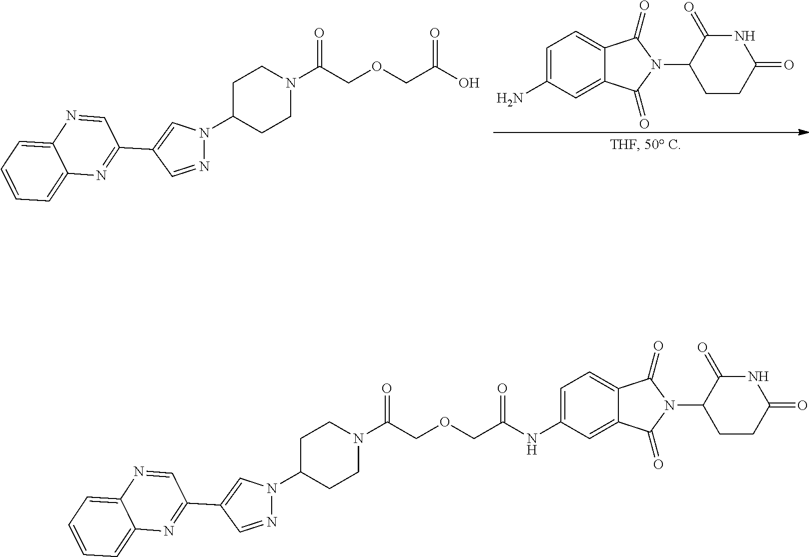 [SMILES: O=C(O)COCC(=O)N1CCC(n2cc(-c3cnc4ccccc4n3)cn2)CC1 ; Nc1ccc2c(c1)C(=O)N(C1CCC(=O)NC1=O)C2=O ; O=C1CCC(N2C(=O)c3ccc(NC(=O)COCC(=O)N4CCC(n5cc(-c6cnc7ccccc7n6)cn5)CC4)cc3C2=O)C(=O)N1]

To a solution of 5-amino-2-(2,6-dioxopiperidin-3-yl)isoindoline-1,3-dione (22 mg, 0.08 mmol) in THF (3 mL) was added 2-(2-oxo-2-(4-(4-(quinoxalin-2-yl)-1H-pyrazol-1-yl)piperidin-1-yl)ethoxy)acetyl chloride (31 mg) in THF (1 mL) at rt. After the reaction was stirred at 60° C. for 1 h, it was poured into water (10 mL) and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (5.8 mg, 12% yield) as a light-yellow solid. MS (ESI) m/z=651.3 [M+H]⁺.

### Example 301. 2-(2,6-Dioxopiperidin-3-yl)-5-(4-(4-(4-(quinoxalin-2-yl)-1H-pyrazol-1-yl)piperidin-1-yl)butyl)isoindoline-1,3-dione (GS-343)

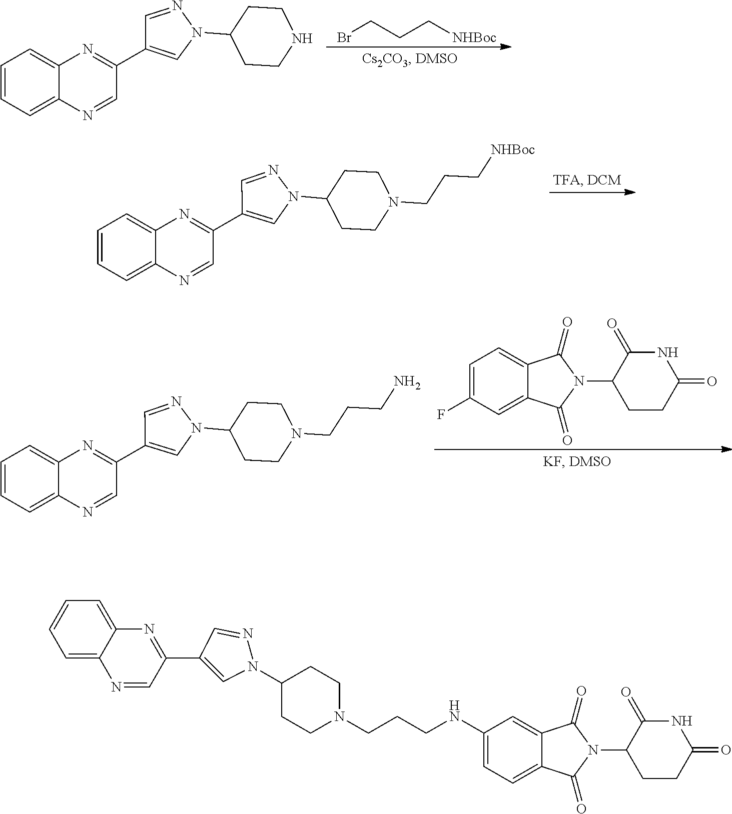

GS-343 was synthesized following the standard procedures for preparing GS-326 (2.8 mg, 22% yield) as a light-yellow solid. MS (ESI) m/z=593.5 [M+H]⁺.

### Example 302. 2-(2,6-Dioxopiperidin-3-yl)-5-((3-(3-(4-(pyridin-2-yl)piperazin-1-yl)pyrrolidin-1-yl)propyl)amino)isoindoline-1,3-dione (GS-344)

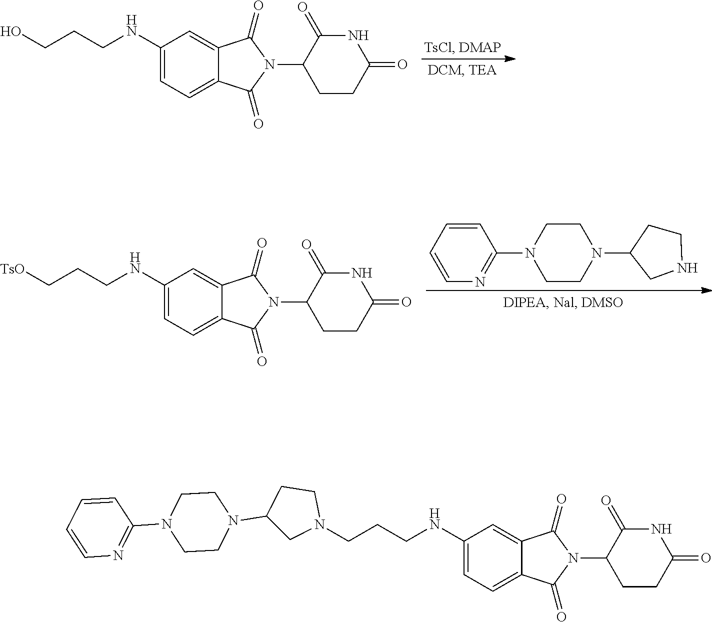

GS-344 was synthesized following the standard procedures for preparing GS-062 (3.5 mg, 65% yield) as a white solid. MS (ESI) m/z=546.4 [M+H]⁺.

### Example 303. N-((2-(2,6-Dioxopiperidin-3-yl)-1,3-dioxoisoindolin-5-yl)methyl)-2-(3-(4-(pyridin-2-yl)piperazin-1-yl)azetidin-1-yl)acetamide (GS-345)

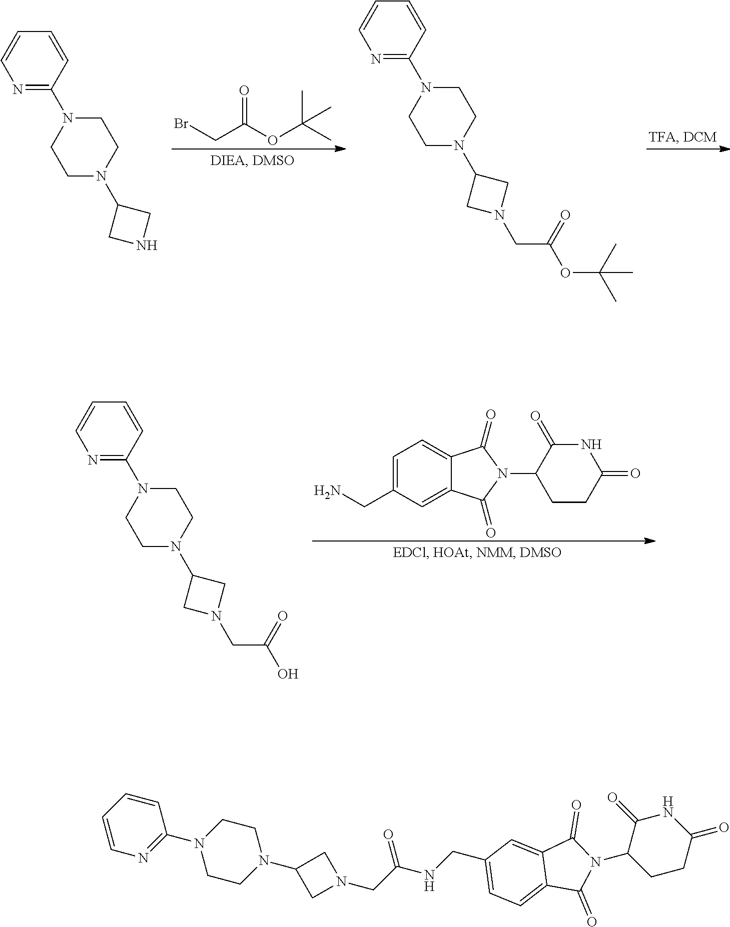

GS-345 was synthesized following the standard procedures for preparing GS-316 (8.2 mg, 45% yield) as a light-yellow solid. MS (ESI) m/z=546.3 [M+H]⁺.

### Example 304. N-((2-(2,6-Dioxopiperidin-3-yl)-1,3-dioxoisoindolin-5-yl)methyl)-2-(3-(4-(quinoxalin-2-yl)-1H-pyrazol-1-yl)piperidin-1-yl)acetamide (GS-346)

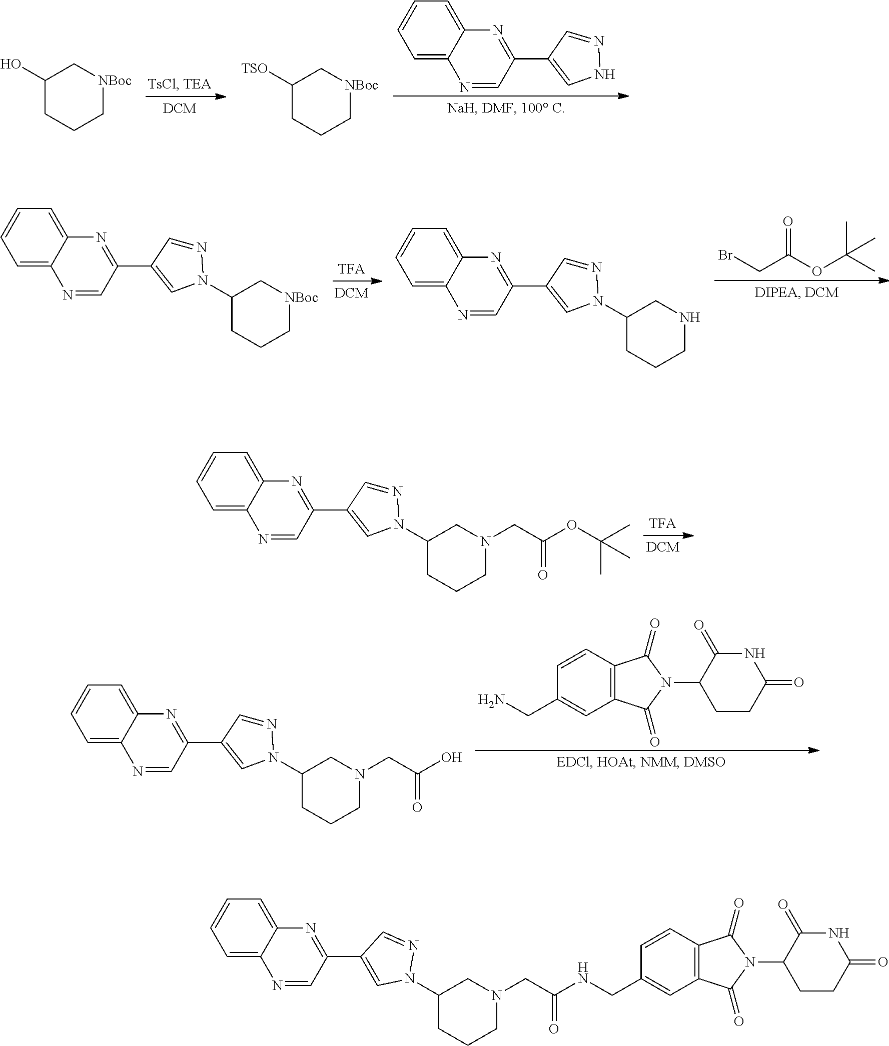

Step 1. Synthesis of tert-butyl 3-(tosyloxy)piperidine-1-carboxylate

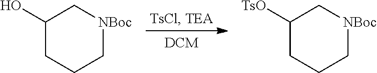 [SMILES: CC(C)(C)OC(=O)N1CCCC(O)C1 ; Cc1ccc(S(=O)(=O)OC2CCCN(C(=O)OC(C)(C)C)C2)cc1]

To a solution of tert-butyl 3-hydroxypiperidine-1-carboxylate (1.1 g, 5.0 mmol) in DCM (50 mL) were added TsCl (1.4 g, 7.5 mmol) and TEA (2.8 g, 27.3 mmol). After the mixture was stirred at rt for 8 h, it was quenched with water (15 mL) and extracted with DCM (15 mL×2). The organic phase was combined, dried over anhydrous sodium sulfate, filtered, and concentrated. The resulting residue was purified by silica gel chromatography to provide the desired product (1.6 g, 90% yield) as a light-yellow solid. MS (ESI) m/z=356.2 [M+H]⁺.

Step 2. Synthesis of tert-butyl 3-(4-(quinoxalin-2-yl)-1H-pyrazol-1-yl)piperidine-1-carboxylate

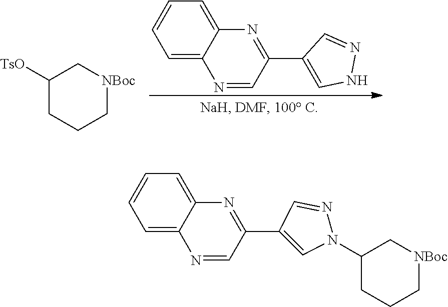 [SMILES: Cc1ccc(S(=O)(=O)OC2CCCN(C(=O)OC(C)(C)C)C2)cc1 ; c1ccc2nc(-c3cn[nH]c3)cnc2c1 ; CC(C)(C)OC(=O)N1CCCC(n2cc(-c3cnc4ccccc4n3)cn2)C1]

To a solution of 2-(1H-pyrazol-4-yl)quinoxaline (80 mg, 0.41 mmol) in DMF (2 mL) was added NaH (32.62 mg, 0.81 mmol, 60% in mineral oil) at 0° C. After stirring at 0° C. for 30 min, tert-butyl 3-(p-tolylsulfonyloxy)piperidine-1-carboxylate (173.91 mg, 0.49 mmol) was added and the solution was heated at 100° C. for 2 h. The reaction mixture was cooled to rt, filtered and purified by reverse-phase chromatography to provide the desired product (80 mg, 0.21 mmol, 52% yield) as a yellow solid. MS (ESI) m/z=380.2 [M+H]⁺.

Step 3. Synthesis of 2-(1-(piperidin-3-yl)-1H-pyrazol-4-yl)quinoxaline

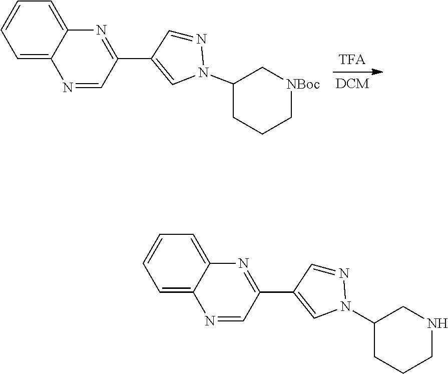 [SMILES: CC(C)(C)OC(=O)N1CCCC(n2cc(-c3cnc4ccccc4n3)cn2)C1 ; c1ccc2nc(-c3cnn(C4CCCNC4)c3)cnc2c1]

To a solution of tert-butyl 3-(4-quinoxalin-2-ylpyrazol-1-yl)piperidine-1-carboxylate (75 mg, 0.19 mmol) in DCM (2 mL) was added TFA (1 mL). The reaction mixture was stirred at rt for 1 h, before it was concentrated and purified by reverse-phase chromatography to provide the desired product (40 mg, 72% yield) as a light-yellow solid. (ESI) m/z=280.2 [M+H]⁺.

The remaining steps were performed according to the standard procedures for preparing GS-316 to provide the desired product (2.4 mg, 23% yield) as a light-yellow solid. MS (ESI) m/z=607.3 [M+H]⁺.

### Example 305. 2-(2,6-Dioxopiperidin-3-yl)-5-((3-(3-(4-(quinoxalin-2-yl)-1H-pyrazol-1-yl)piperidin-1-yl)propyl)amino)isoindoline-1,3-dione (GS-347)

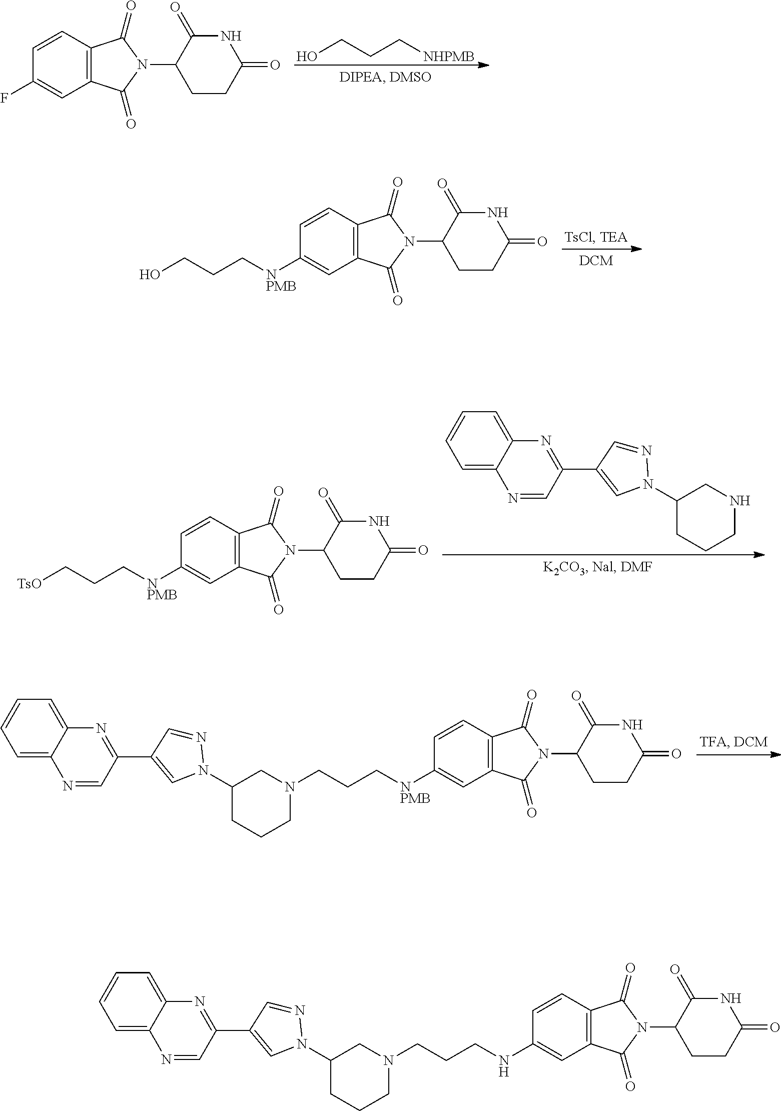

Step 1. Synthesis of 2-(2,6-dioxopiperidin-3-yl)-5-((3-hydroxypropyl)(4-methoxybenzyl)amino)isoindoline-1,3-dione

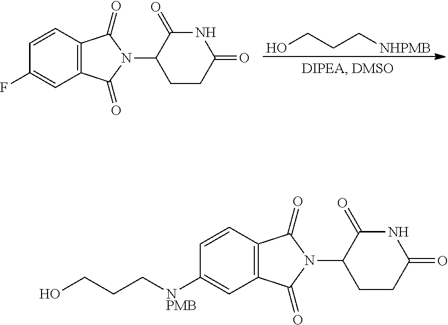

To a solution of 2-(2,6-dioxopiperidin-3-yl)-5-fluoroisoindoline-1,3-dione (100 mg, 0.36 mmol) in DMSO (5 mL) were added 3-((4-methoxybenzyl)amino)propan-1-ol (106 mg, 0.54 mmol) and DIPEA (140 mg, 1.1 mmol). The reaction mixture was stirred at 100° C. for 4 h. After cooling down to rt, the solution was poured into water (20 mL) and extracted with ethyl acetate (20 mL×3). The combined organic phase was concentrated and purified by reverse-phase chromatography to provide the desired product (60.5 mg, 37% yield) as a light-yellow solid. MS (ESI) m/z=452.2 [M+H]⁺.

Step 2. Synthesis of 3-((2-(2,6-dioxopiperidin-3-yl)-1,3-dioxoisoindolin-5-yl)(4-methoxybenzyl)amino)propyl 4-methylbenzenesulfonate

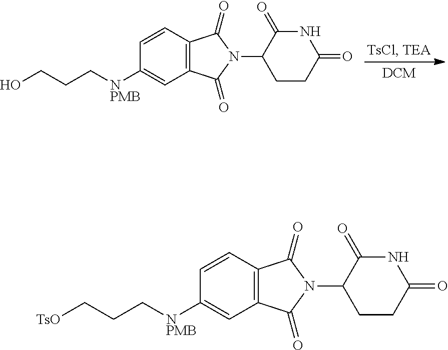

To a solution of 2-(2,6-dioxopiperidin-3-yl)-5-((3-hydroxypropyl)(4-methoxybenzyl)amino)isoindoline-1,3-dione (60.5 mg, 0.13 mmol) in DCM (5 mL) were added TsCl (37.9 mg, 0.2 mmol) and TEA (40.3 mg, 0.4 mmol). The reaction mixture was stirred at rt for 4 h, before it was concentrated and purified by reverse-phase chromatography to provide the desired product (30.1 mg, 37% yield) as a light-yellow solid. MS (ESI) m/z=606.2 [M+H]⁺.

Step 3. Synthesis of 2-(2,6-dioxopiperidin-3-yl)-5-((4-methoxybenzyl)(3-(3-(4-(quinoxalin-2-yl)-1H-pyrazol-1-yl)piperidin-1-yl)propyl)amino)isoindoline-1,3-dione

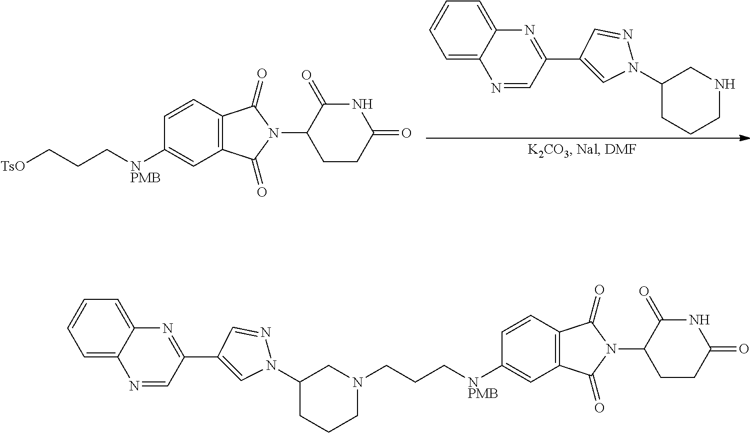

To a solution of 2-(2,6-dioxopiperidin-3-yl)-5-((3-hydroxypropyl)(4-methoxybenzyl)amino)isoindoline-1,3-dione (17.0 mg, 0.03 mmol) in DMF (5 mL) were added 2-(1-(piperidin-3-yl)-1H-pyrazol-4-yl)quinoxaline (9.4 mg, 0.03 mmol), K₂CO₃ (11.6 mg, 0.8 mmol) and NaI (4.2 mg, 0.03 mmol). The reaction mixture was stirred at 80° C. for 1 h, before it was concentrated and purified by reverse-phase chromatography to provide the desired product (4.1 mg, 20% yield) as a light-yellow solid. MS (ESI) m/z=713.4 [M+H]⁺

Step 4. Synthesis of 2-(2,6-dioxopiperidin-3-yl)-5-((3-(3-(4-(quinoxalin-2-yl)-1H-pyrazol-1-yl)piperidin-1-yl)propyl)amino)isoindoline-1,3-dione

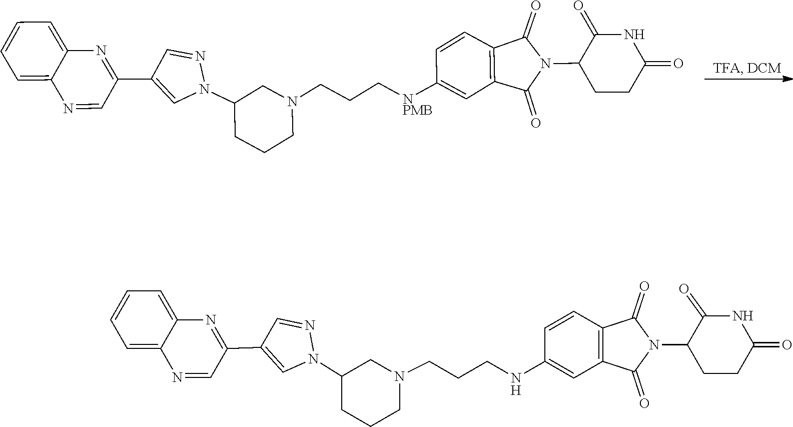

A solution of 2-(2,6-dioxopiperidin-3-yl)-5-((4-methoxybenzyl)(3-(3-(4-(quinoxalin-2-yl)-1H-pyrazol-1-yl)piperidin-1-yl)propyl)amino)isoindoline-1,3-dione (4.1 mg, 0.005 mmol) in DCM (2 mL) and TFA (1 mL) was stirred at rt for 4 h. The mixture was concentrated and purified by reverse-phase chromatography to provide the desired product (2.4 mg, 70% yield) as a light-yellow solid. MS (ESI) m/z=593.3 [M+H]⁺.

### Example 306. 2-(2,6-Dioxopiperidin-3-yl)-5-((6-(4-fluoro-4-phenylpiperidin-1-yl)-6-oxohexyl)amino)isoindoline-1,3-dione (GS-348)

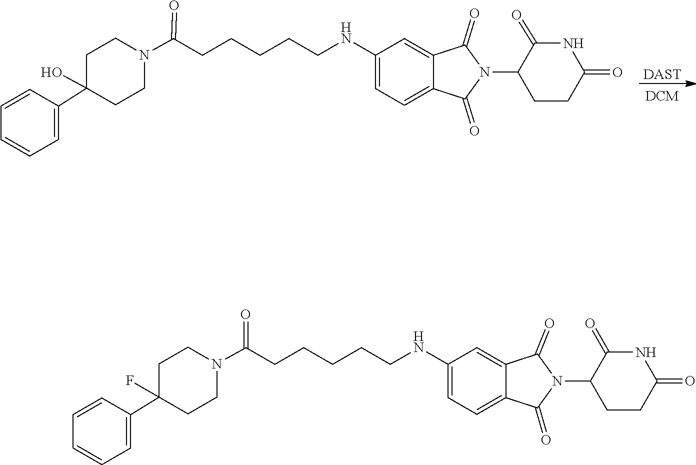

To a solution of 2-(2,6-dioxopiperidin-3-yl)-5-((6-(4-hydroxy-4-phenylpiperidin-1-yl)-6-oxohexyl)amino)isoindoline-1,3-dione (10 mg, 0.02 mmol) in DCM (10 mL) was added DAST (32 mg, 0.2 mmol) at 0° C. The reaction mixture was stirred at rt for 30 min, before it was quenched with saturated aqueous NaHCO₃ and extracted with DCM (5 mL×3). The combined organic phase was washed with brine, dried over Na₂SO₄, filtered, and concentrated. The resulting residue was purified by reverse-phase chromatography to provide the desired product (2.1 mg, 21% yield) as a yellow solid. MS (ESI) m/z=549.3 [M+H]⁺.

### Example 307. 1-((2-(2,6-Dioxopiperidin-3-yl)-1,3-dioxoisoindolin-5-yl)methyl)-3-(3-(4-(quinoxalin-2-yl)-1H-pyrazol-1-yl)cyclohexyl)urea (GS-349)

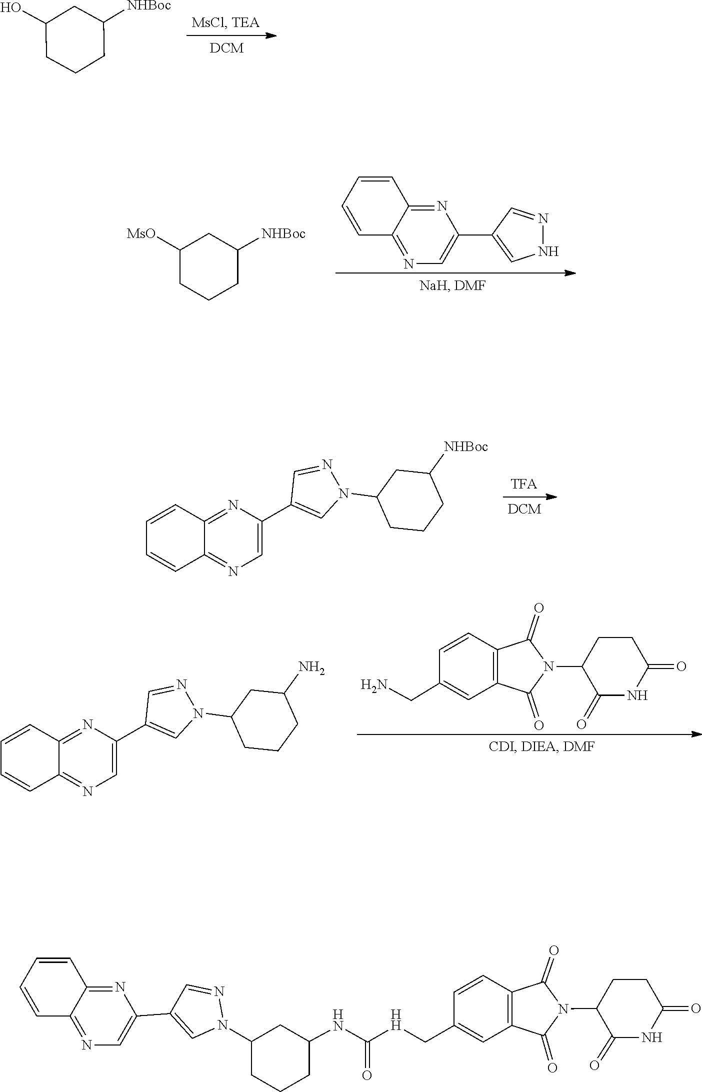

Step 1. Synthesis of 3-((tert-butoxycarbonyl)amino)cyclohexyl methanesulfonate

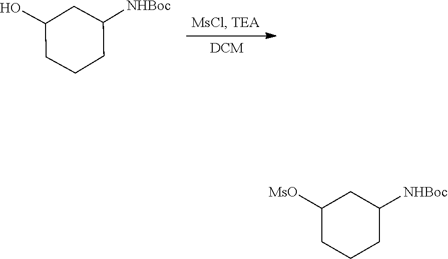 [SMILES: CC(C)(C)OC(=O)NC1CCCC(O)C1 ; CC(C)(C)OC(=O)NC1CCCC(OS(C)(=O)=O)C1]

To a solution of tert-butyl (3-hydroxycyclohexyl)carbamate (300 mg, 1.4 mmol) in DCM (10 mL) were added TEA (707 mg, 7 mmol) and MsCl (318 mg, 2.8 mmol) dropwise at 0° C. The reaction mixture was stirred at 0° C. for 2 h, before it was quenched with water and extracted with DCM (3×10 mL). The combined organic phase was washed with brine dried over anhydrous sodium sulfate, filtered, and concentrated. The resulting residue was purified by silica gel flash chromatography (petroleum ether/ethyl acetate=5:1) to provide the desired product (320 mg, 78%) as a white solid. MS (ESI) m/z=294.2 [M+H]⁺.

Step 2. Synthesis of tert-butyl (3-(4-(quinoxalin-2-yl)-1H-pyrazol-1-yl)cyclohexyl)carbamate

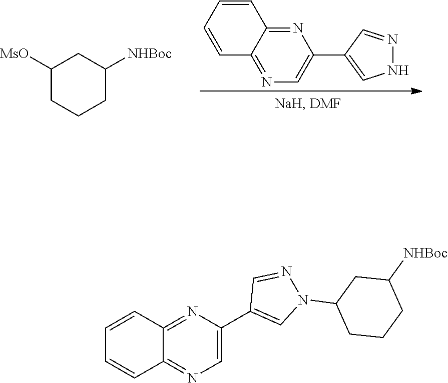 [SMILES: CC(C)(C)OC(=O)NC1CCCC(OS(C)(=O)=O)C1 ; c1ccc2nc(-c3cn[nH]c3)cnc2c1 ; CC(C)(C)OC(=O)NC1CCCC(n2cc(-c3cnc4ccccc4n3)cn2)C1]

To a solution of 2-(1H-pyrazol-4-yl)quinoxaline (30 mg, 0.15 mmol) in DMF (3 mL) was added NaH (11 mg, 0.45 mmol) at 0° C. under N₂. After stirring at 0° C. for 20 min, 3-((tert-butoxycarbonyl)amino)cyclohexyl methanesulfonate (175 mg, 0.6 mmol) was added. The mixture was stirred at 100° C. for another 16 h. After cooling down to rt, the reaction was quenched with water (10 mL) and extracted with ethyl acetate (3×10 mL). The combined organic phase was washed with brine, dried over anhydrous sodium sulfate, filtered, and concentrated under reduced pressure. The resulting residue was purified by silica gel column chromatography (petroleum ether/ethyl acetate=3:1) to provide the desired product (20 mg, 33%) as a yellow oil. MS (ESI) m/z=394.3 [M+H]⁺.

Step 3. Synthesis of 3-(4-(quinoxalin-2-yl)-1H-pyrazol-1-yl)cyclohexan-1-amine

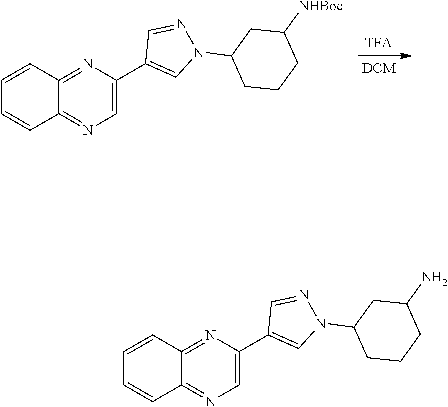 [SMILES: CC(C)(C)OC(=O)NC1CCCC(n2cc(-c3cnc4ccccc4n3)cn2)C1 ; NC1CCCC(n2cc(-c3cnc4ccccc4n3)cn2)C1]

A solution of tert-butyl (3-(4-(quinoxalin-2-yl)-1H-pyrazol-1-yl)cyclohexyl)carbamate (20 mg, 0.05 mmol) in DCM (2 mL) and TFA (1 mL) was stirred at rt for 2 h. Then the reaction mixture was concentrated and purified by reverse phase chromatography to provide the desired product (11 mg, 74%) as a yellow oil. MS (ESI) m/z=294.2 [M+H]⁺.

Step 4. Synthesis of 1-((2-(2,6-dioxopiperidin-3-yl)-1,3-dioxoisoindolin-5-yl)methyl)-3-(3-(4-(quinoxalin-2-yl)-1H-pyrazol-1-yl)cyclohexyl)urea

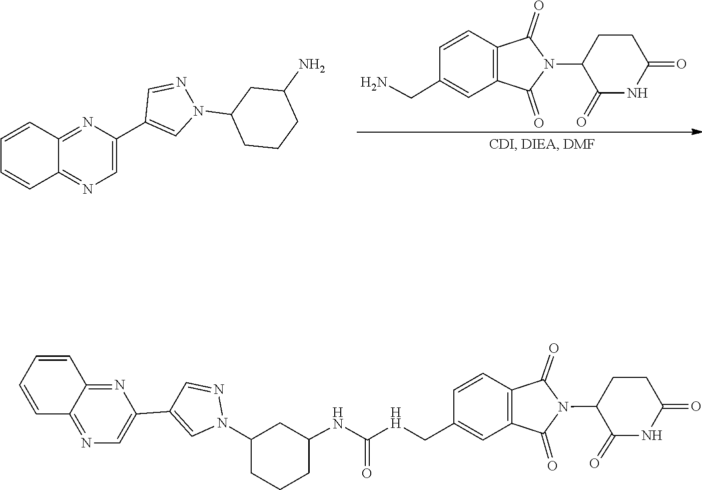

To a solution of 5-(aminomethyl)-2-(2,6-dioxopiperidin-3-yl)isoindoline-1,3-dione (10 mg, 0.03 mmol) in DMF (1 mL) were added DIEA (19 mg, 0.15 mmol) and CDI (10 mg, 0.06 mmol). After stirring at rt for 2 h, 3-(4-(quinoxalin-2-yl)-1H-pyrazol-1-yl)cyclohexan-1-amine (11 mg, 0.03 mmol) was added. The mixture was stirred at 60° C. for another 16 h, before it was purified by reverse phase chromatography to provide the desired product (5 mg, 23%) as a yellow solid. MS (ESI) m/z=607.3 [M+H]⁺.

### Example 308. 2-(2,6-Dioxopiperidin-3-yl)-5-((3-(3-(4-(pyridin-2-yl)piperazin-1-yl)piperidin-1-yl)propyl)amino)isoindoline-1,3-dione (GS-350)

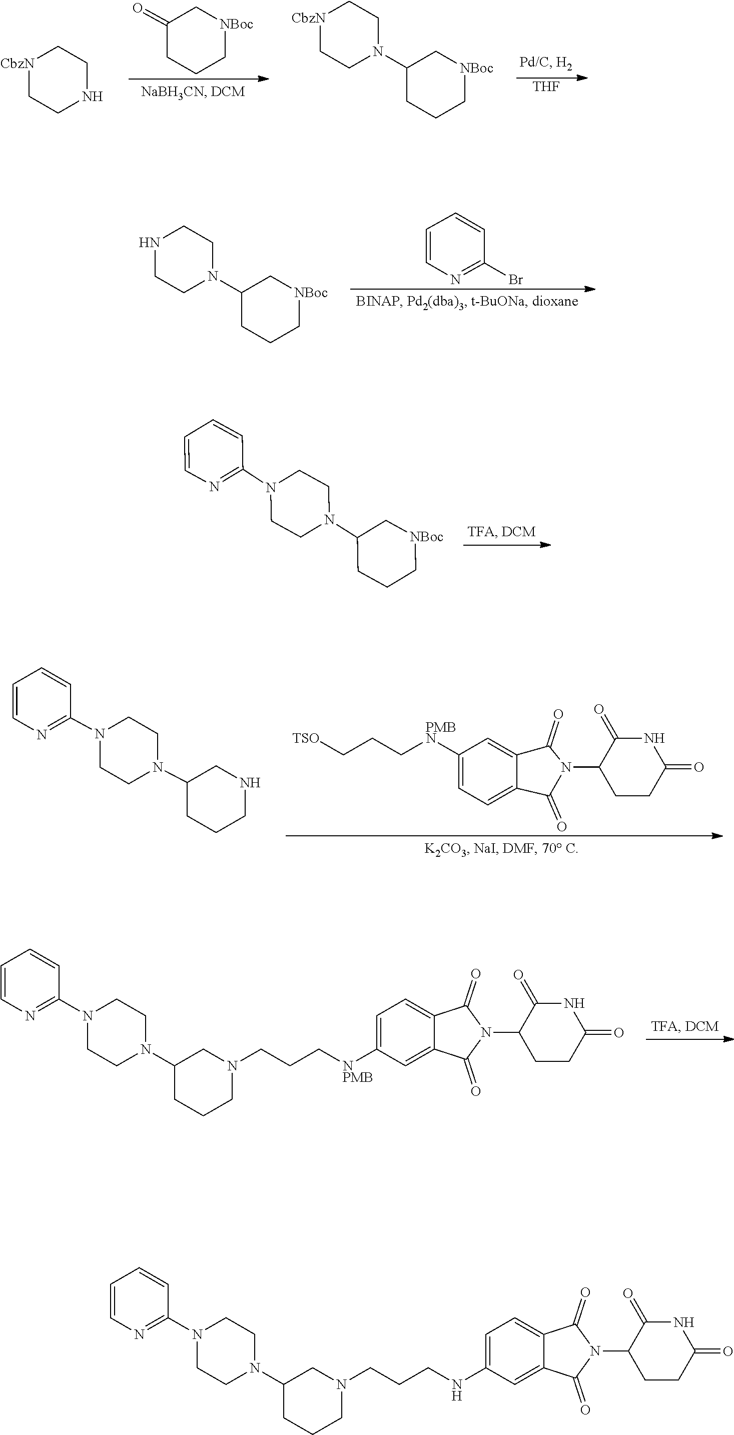

Step 1. Synthesis of benzyl 4-(1-(tert-butoxycarbonyl)piperidin-3-yl)piperazine-1-carboxylate

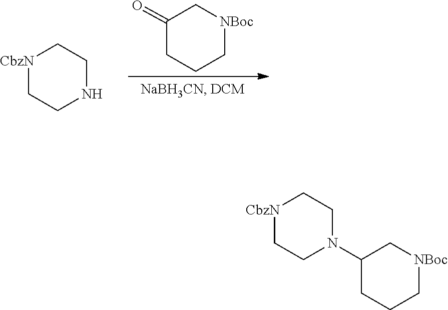 [SMILES: O=C(OCc1ccccc1)N1CCNCC1 ; CC(C)(C)OC(=O)N1CCCC(=O)C1 ; CC(C)(C)OC(=O)N1CCCC(N2CCN(C(=O)OCc3ccccc3)CC2)C1]

To a solution of benzyl piperazine-1-carboxylate (100 mg, 0.43 mmol) in DCM (4 mL) were added tert-butyl 3-oxopiperidine-1-carboxylate (135 mg, 0.68 mmol) and NaBH₃CN (85.9 mg, 1.4 mmol). The reaction mixture was stirred at rt for 4 h, before it was quenched with water (5 mL) and extracted with DCM (10 mL×3). The combined organic phase was dried over anhydrous sodium sulfate, filtered, and concentrated. The resulting residue was purified by reverse-phase chromatography to provide the desired product (150 mg, 82% yield) as a light-yellow solid. MS (ESI) m/z=404.3 [M+H]⁺.

Step 2. Synthesis of tert-butyl 3-(piperazin-1-yl)piperidine-1-carboxylate

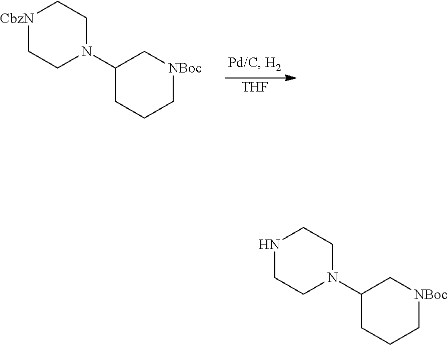 [SMILES: CC(C)(C)OC(=O)N1CCCC(N2CCN(C(=O)OCc3ccccc3)CC2)C1 ; CC(C)(C)OC(=O)N1CCCC(N2CCNCC2)C1 ; "THF"]

To a solution of benzyl 4-(1-(tert-butoxycarbonyl)piperidin-3-yl)piperazine-1-carboxylate (150 mg, 0.37 mmol) in THF (4 mL) was added 10% Pd/C (20 mg). The reaction mixture was purged with hydrogen and stirred at rt for 4 h under hydrogen balloon. The mixture was filtered through Celite. The filtrate was concentrated and purified by reverse-phase chromatography to provide the desired product (100 mg, 99% yield) as a light-yellow solid. MS (ESI) m/z=270.2 [M+H]⁺.

Step 3. Synthesis of tert-butyl 3-(4-(pyridin-2-yl)piperazin-1-yl)piperidine-1-carboxylate

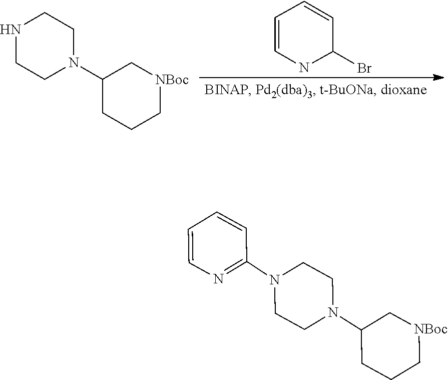 [SMILES: CC(C)(C)OC(=O)N1CCCC(N2CCNCC2)C1 ; BrC1C=CC=CN1 ; CC(C)(C)OC(=O)N1CCCC(N2CCN(c3ccccn3)CC2)C1]

To a solution of tert-butyl 3-(piperazin-1-yl)piperidine-1-carboxylate (100 mg, 0.37 mmol) in dioxane (10 mL) were added 2-bromopyridine (86.9 mg, 0.56 mmol), Pd₂(dba)₃ (17.2 mg, 0.02 mmol), BINAP (23.1 mg, 0.4 mmol) and t-BuONa (71.1 mg, 0.75 mmol) under inert atmosphere. The reaction mixture was stirred at 100° C. for 16 h. After cooling down to rt, the mixture was filtered and concentrated under reduced pressure. The resulting residue was purified by reverse-phase chromatography to provide the desired product (60 mg, 46% yield) as a light-yellow solid. MS (ESI) m/z=347.2 [M+H]⁺.

Step 4. Synthesis of 1-(piperidin-3-yl)-4-(pyridin-2-yl)piperazine

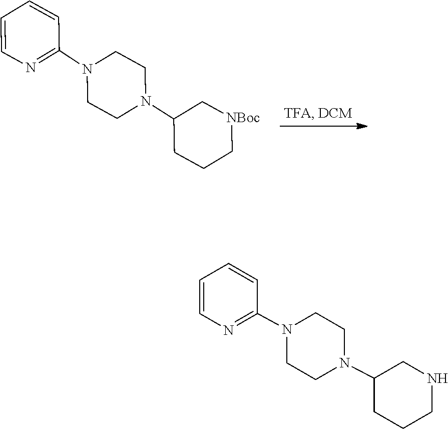 [SMILES: CC(C)(C)OC(=O)N1CCCC(N2CCN(c3ccccn3)CC2)C1 ; c1ccc(N2CCN(C3CCCNC3)CC2)nc1]

A solution of tert-butyl 3-(4-(pyridin-2-yl)piperazin-1-yl)piperidine-1-carboxylate (60 mg, 0.17 mmol) in DCM (4 mL) and TFA (2 mL) was stirred at rt for 1 h. Then the mixture was concentrated and purified by reverse-phase chromatography to provide the desired product (42 mg, 98% yield) as a light-yellow solid. MS (ESI) m/z=247.2 [M+H]⁺.

The remaining steps were performed according to the standard procedures for preparing GS-347 to provide the desired product (2.7 mg, 40% yield) as a light-yellow solid. MS (ESI) m/z=560.4 [M+H]⁺.

### Example 309. 2-(2,6-Dioxopiperidin-3-yl)-5-((2-((3-(4-(4-(quinoxalin-2-yl)-1H-pyrazol-1-yl)piperidin-1-yl)phenyl)amino)ethyl)amino)isoindoline-1,3-dione (GS-351)

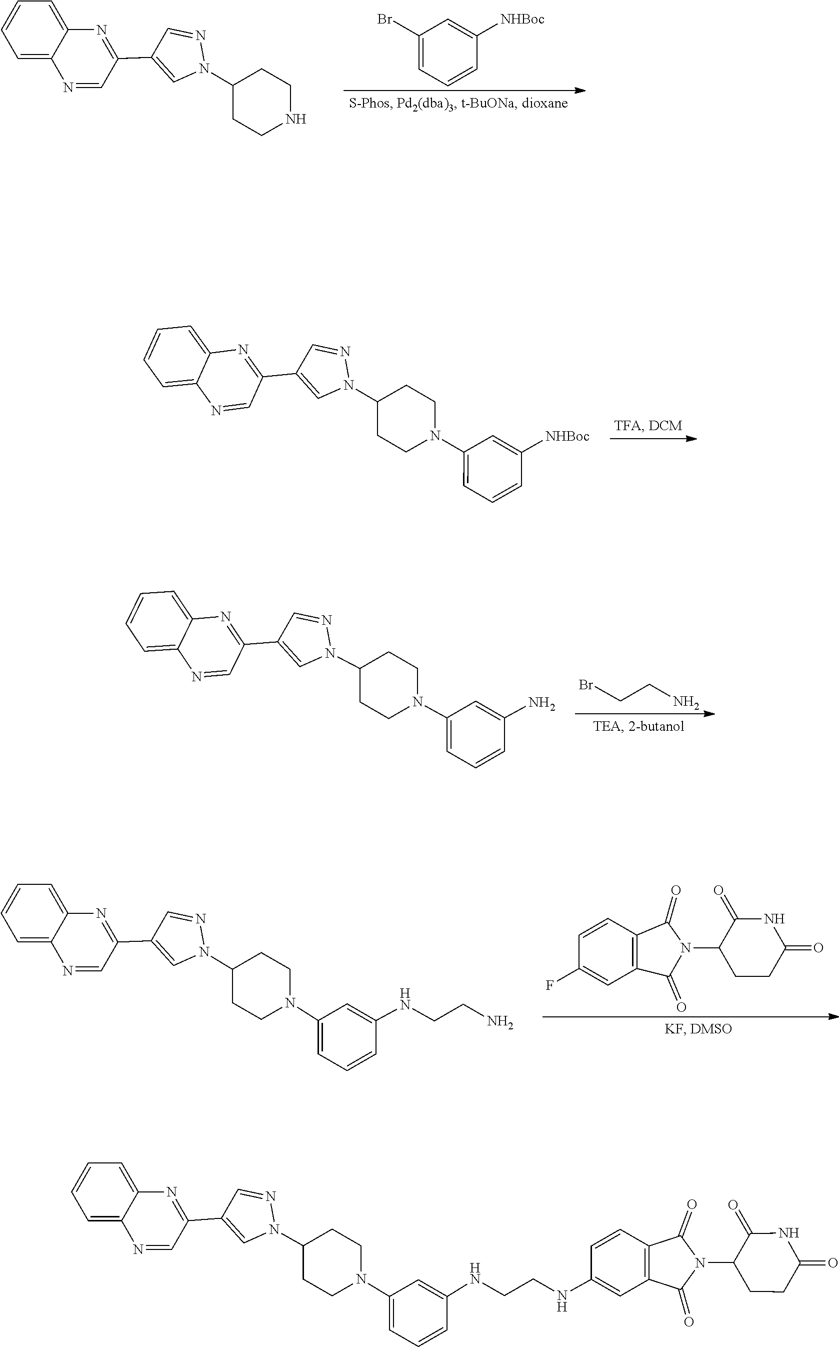

Step 1. Synthesis of tert-butyl (3-(4-(4-(quinoxalin-2-yl)-1H-pyrazol-1-yl)piperidin-1-yl)phenyl)carbamate

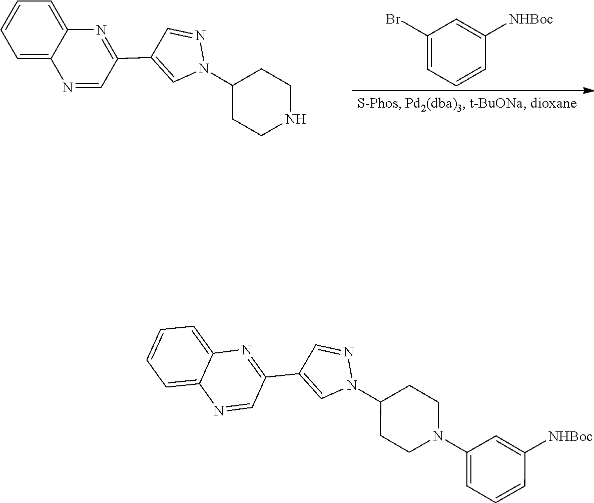 [SMILES: c1ccc2nc(-c3cnn(C4CCNCC4)c3)cnc2c1 ; CC(C)(C)OC(=O)Nc1cccc(Br)c1 ; CC(C)(C)OC(=O)Nc1cccc(N2CCC(n3cc(-c4cnc5ccccc5n4)cn3)CC2)c1]

To a solution of 2-(1-(piperidin-4-yl)-1H-pyrazol-4-yl)quinoxaline (see step 2 of GS-005 for synthesis, 100 mg, 0.36 mmol) in dioxane (10 mL) were added tert-butyl (3-bromophenyl)carbamate (147 mg, 0.53 mmol), Pd₂(dba)₃ (16.3 mg, 0.02 mmol), S-Phos (14.7 mg, 0.4 mmol) and t-BuONa (104 mg, 1.1 mmol) at rt under inert atmosphere. The reaction mixture was stirred at 100° C. for 8 h. The mixture was filtered through Celite. The filtrate was concentrated and purified by reverse-phase chromatography to provide the desired product (64.7 mg, 38% yield) as a light-yellow solid. MS (ESI) m/z=471.3 [M+H]⁺.

Step 2. Synthesis of 3-(4-(4-(quinoxalin-2-yl)-1H-pyrazol-1-yl)piperidin-1-yl)aniline

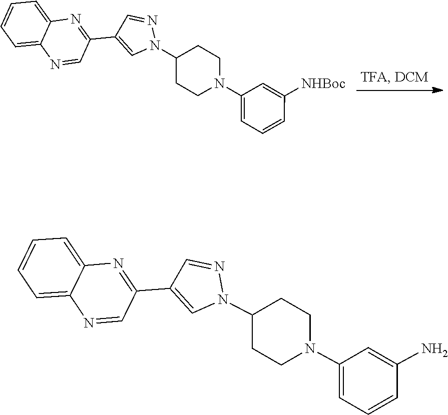 [SMILES: CC(C)(C)OC(=O)Nc1cccc(N2CCC(n3cc(-c4cnc5ccccc5n4)cn3)CC2)c1 ; Nc1cccc(N2CCC(n3cc(-c4cnc5ccccc5n4)cn3)CC2)c1]

A solution of tert-butyl 3-(4-(pyridin-2-yl)piperazin-1-yl)piperidine-1-carboxylate (64.7 mg, 261 mmol) in DCM (4 mL) and TFA (2 mL) was stirred at rt for 4 h. The mixture was concentrated and purified by reverse-phase chromatography to provide the desired product (49.2 mg, 96% yield) as a light-yellow solid. MS (ESI) m/z=371.3 [M+H]⁺.

Step 3. Synthesis of N′-(3-(4-(4-(quinoxalin-2-yl)-1H-pyrazol-1-yl)piperidin-1-yl)phenyl)ethane-1,2-diamine

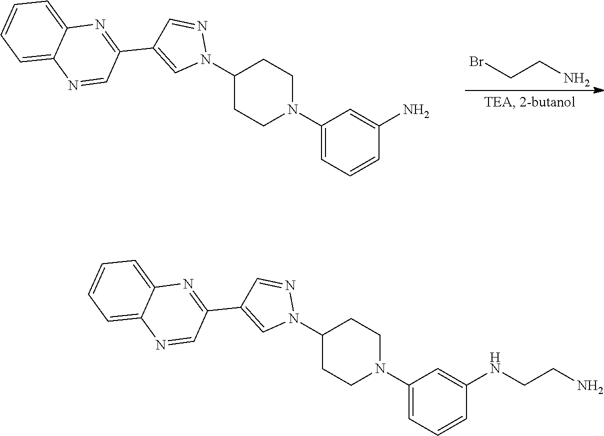

To a solution of 3-(4-(4-(quinoxalin-2-yl)-1H-pyrazol-1-yl)piperidin-1-yl)aniline (25 mg, 0.07 mmol) in 2-butanol (5 mL) were added 2-bromoethylamine hydrobromide (69.3 mg, 0.33 mmol) and TEA (68.2 mg, 0.68 mmol). The reaction mixture was stirred at reflux under inert atmosphere for 48 h, before it was concentrated and purified by reverse-phase chromatography to provide the desired product (12.4 mg, 44% yield) as a light-yellow solid. MS (ESI) m/z=414.4 [M+H]⁺

Step 4. Synthesis of 2-(2,6-dioxopiperidin-3-yl)-5-((2-((3-(4-(4-(quinoxalin-2-yl)-1H-pyrazol-1-yl)piperidin-1-yl)phenyl)amino)ethyl)amino)isoindoline-1,3-dione

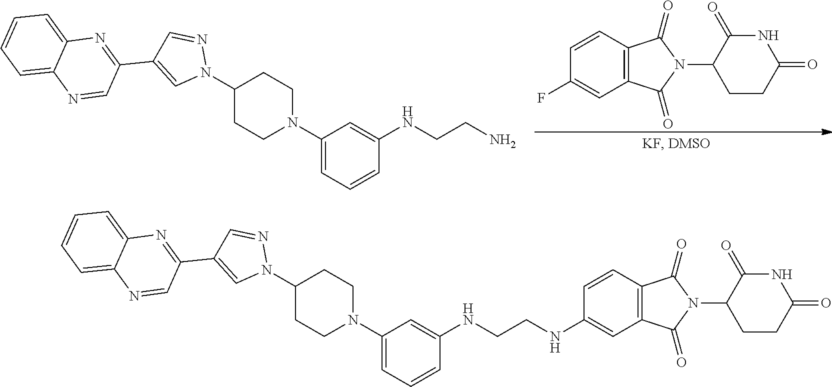 [SMILES: NCCNc1cccc(N2CCC(n3cc(-c4cnc5ccccc5n4)cn3)CC2)c1 ; O=C1CCC(N2C(=O)c3ccc(F)cc3C2=O)C(=O)N1 ; O=C1CCC(N2C(=O)c3ccc(NCCNc4cccc(N5CCC(n6cc(-c7cnc8ccccc8n7)cn6)CC5)c4)cc3C2=O)C(=O)N1]

To a solution of N′-(3-(4-(4-(quinoxalin-2-yl)-1H-pyrazol-1-yl)piperidin-1-yl)phenyl)ethane-1,2-diamine (12.4 mg, 0.03 mmol) in DMSO (2 mL) were added 2-(2,6-dioxopiperidin-3-yl)-5-fluoroisoindoline-1,3-dione (12.4 mg, 0.04 mmol) and KF (5.2 mg, 0.09 mmol) at rt. Then the mixture was stirred at 130° C. under microwave irradiation for 0.25 h. After cooling down to rt, the mixture was purified by reverse-phase chromatography to provide the desired product (3.2 mg, 15% yield) as a light-yellow solid. MS (ESI) m/z=670.4 [M+H]⁺.

### Example 310. 2-(2,6-Dioxopiperidin-3-yl)-5-(7-(4-(quinoxalin-2-yl)-1H-pyrazol-1-yl)heptyl)isoindoline-1,3-dione (GS-352)

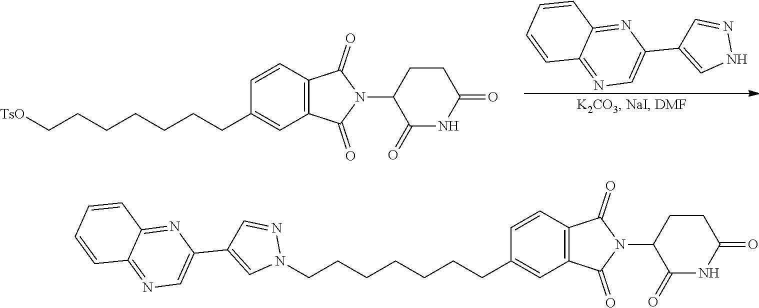 [SMILES: Cc1ccc(S(=O)(=O)OCCCCCCCc2ccc3c(c2)C(=O)N(C2CCC(=O)NC2=O)C3=O)cc1 ; c1ccc2nc(-c3cn[nH]c3)cnc2c1 ; O=C1CCC(N2C(=O)c3ccc(CCCCCCCn4cc(-c5cnc6ccccc6n5)cn4)cc3C2=O)C(=O)N1]

GS-352 was synthesized following the standard procedures for preparing GS-347 (2.6 mg, 10% yield) as a light-yellow solid. MS (ESI) m/z=551.3 [M+H]⁺.

### Example 311. 2-(2,6-Dioxopiperidin-3-yl)-5-((2-((3-(4-(quinoxalin-2-yl)-1H-pyrazol-1-yl)phenyl)amino)ethyl)amino)isoindoline-1,3-dione (GS-353)

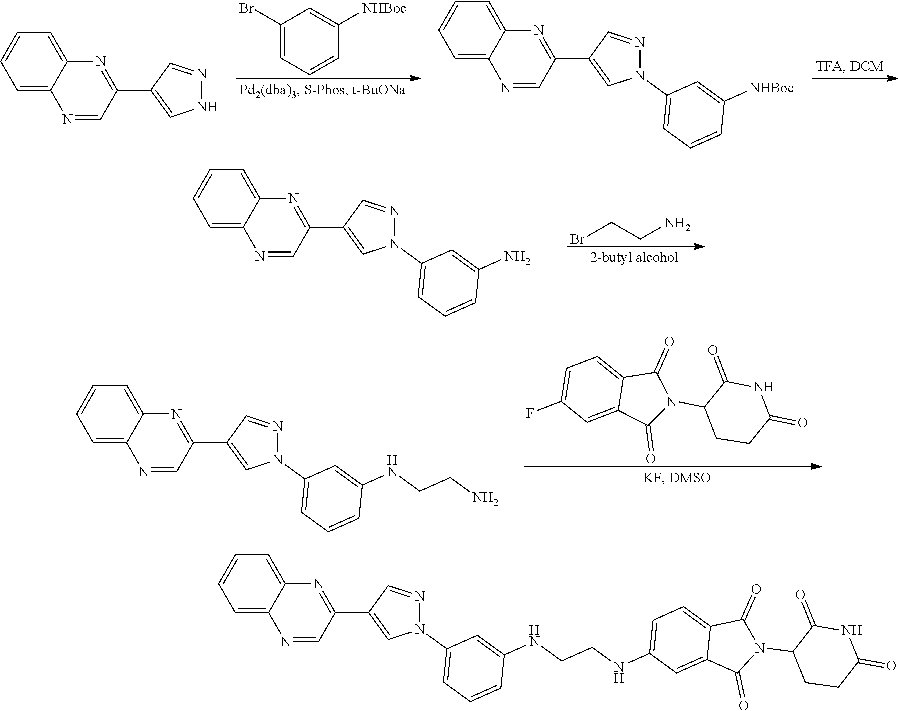

GS-353 was synthesized following the standard procedures for preparing GS-351 (6.8 mg, 42% yield) as a light-yellow solid. MS (ESI) m/z=587.3 [M+H]⁺.

### Example 312. 2-(2,6-Dioxopiperidin-3-yl)-5-((6-(4-(quinoxalin-2-yl)-1H-pyrazol-1-yl)hexyl)oxy)isoindoline-1,3-dione (GS-354)

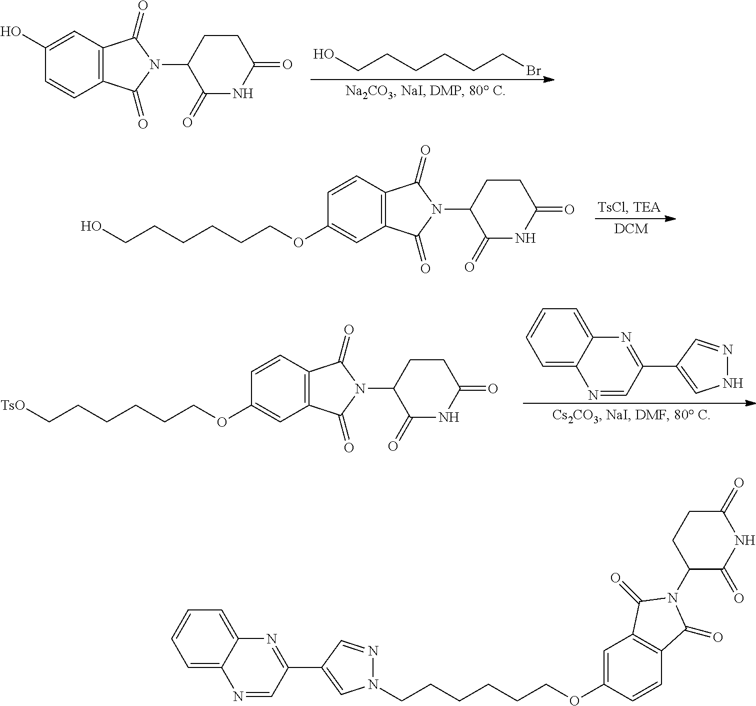

Step 1. Synthesis of 2-(2,6-dioxopiperidin-3-yl)-5-((6-hydroxyhexyl)oxy)isoindoline-1,3-dione

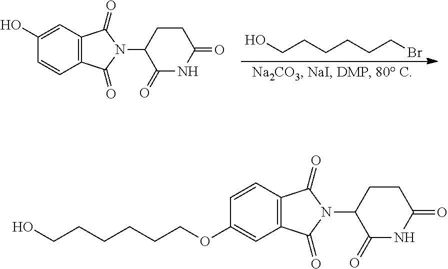 [SMILES: O=C1CCC(N2C(=O)c3ccc(O)cc3C2=O)C(=O)N1 ; OCCCCCCBr ; O=C1CCC(N2C(=O)c3ccc(OCCCCCCO)cc3C2=O)C(=O)N1]

A mixture of 2-(2,6-dioxopiperidin-3-yl)-5-hydroxyisoindoline-1,3-dione (30 mg, 0.11 mmol), 6-

bromohexan-1-ol (23 mg, 0.13 mmol), Na₂CO₃ (23 mg, 0.22 mmol) and NaI (16 mg, 0.11 mmol) in DMF (3 mL) was stirred at 80° C. for 2 h. The reaction mixture was poured into water (10 mL) and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (34 mg, 83% yield) as a light-yellow solid. MS (ESI) m/z=375.1 [M+H]⁺.

Step 2. Synthesis of 6-((2-(2,6-dioxopiperidin-3-yl)-1,3-dioxoisoindolin-5-yl)oxy)hexyl 4-methylbenzenesulfonate

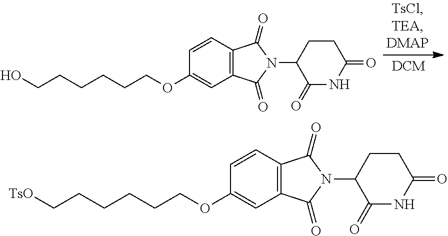

To a solution of 2-(2,6-dioxopiperidin-3-yl)-5-((6-hydroxyhexyl)oxy)isoindoline-1,3-dione (30 mg, 0.08 mmol), TEA (16 mg, 0.16 mmol) and DMAP (2 mg, 0.02 mmol) in DCM (3 mL) was added 4-methylbenzenesulfonyl chloride (19 mg, 0.1 mmol). The mixture was stirred at rt for 2 h, before it was poured into water (10 mL) and extracted with DCM (3×10 mL). The combined organic layers were washed with saturated brine (10 mL), dried over anhydrous sodium sulfate, filtered, and concentrated under reduced pressure. The resulting residue was purified by silica gel column chromatography to provide the desired product (31 mg, 74% yield) as a light-yellow solid. MS (ESI) m/z=529.2 [M+H]⁺.

Step 3. Synthesis of 2-(2,6-dioxopiperidin-3-yl)-5-((6-(4-(quinoxalin-2-yl)-1H-pyrazol-1-yl)hexyl)oxy)isoindoline-1,3-dione

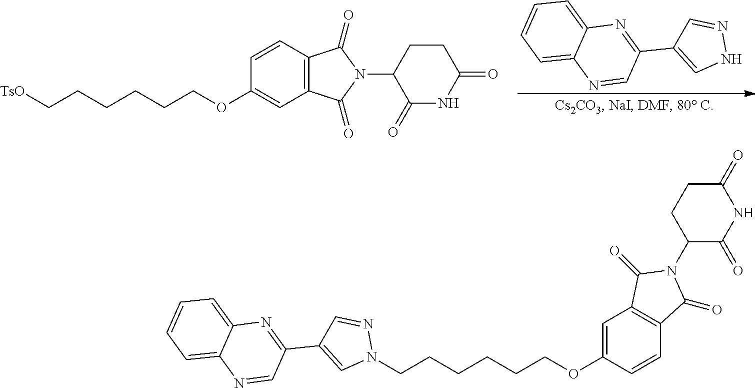 [SMILES: Cc1ccc(S(=O)(=O)OCCCCCCOc2ccc3c(c2)C(=O)N(C2CCC(=O)NC2=O)C3=O)cc1 ; c1ccc2nc(-c3cn[nH]c3)cnc2c1 ; O=C1CCC(N2C(=O)c3ccc(OCCCCCCn4cc(-c5cnc6ccccc6n5)cn4)cc3C2=O)C(=O)N1]

A mixture of 6-((2-(2,6-dioxopiperidin-3-yl)-1,3-dioxoisoindolin-5-yl)oxy)hexyl 4-methylbenzenesulfonate (10 mg, 0.02 mmol), 2-(1H-pyrazol-4-yl)quinoxaline (4 mg, 0.02 mmol), Cs₂CO₃ (13 mg, 0.04 mmol) and NaI (3 mg, 0.02 mmol) in DMF (3 mL) was stirred at 80° C. for 2 h. The reaction mixture was poured into water (10 mL) and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (5.1 mg, 46% yield) as a light-yellow solid. MS (ESI) m/z=553.2 [M+H]⁺.

### Example 313. 2-(2,6-Dioxopiperidin-3-yl)-5-((3-(4-(quinoxalin-2-yl)-1H-pyrazol-1-yl)propyl)amino)isoindoline-1,3-dione (GS-355)

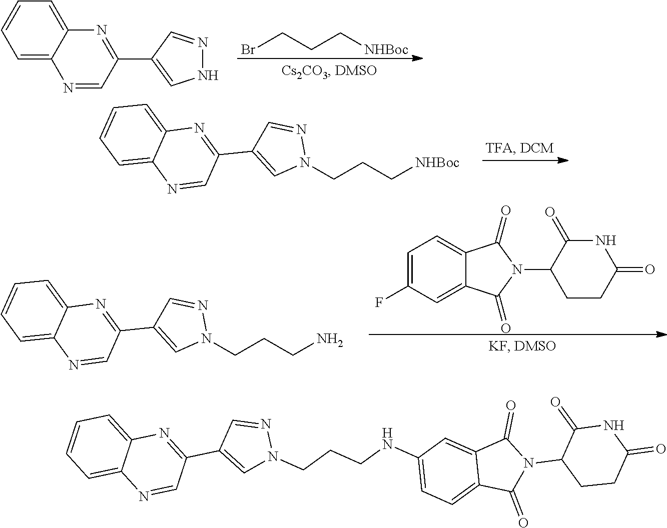

GS-355 was synthesized following the standard procedures for preparing GS-326 (8.2 mg, 45% yield) as a light-yellow solid. MS (ESI) m/z=510.3 [M+H]⁺.

### Example 314. 2-(2,6-Dioxopiperidin-3-yl)-5-((5-(4-(quinoxalin-2-yl)-1H-pyrazol-1-yl)pentyl)amino)isoindoline-1,3-dione (GS-356)

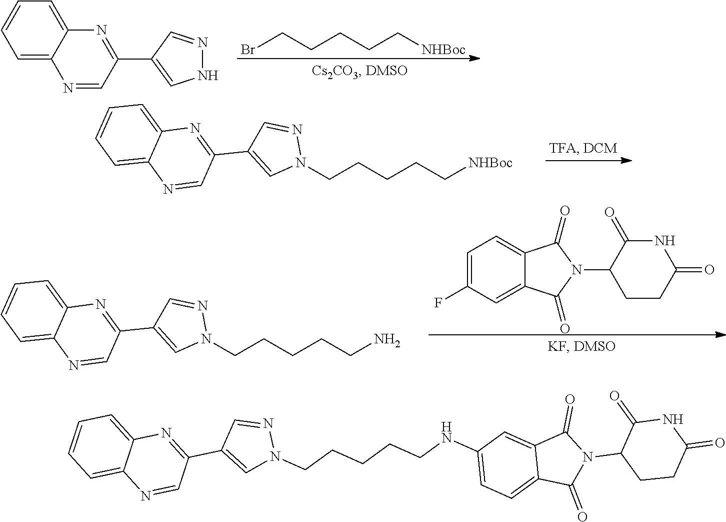

GS-356 was synthesized following the standard procedures for preparing GS-326 (7.5 mg, 42% yield) as a light-yellow solid. MS (ESI) m/z=538.3 [M+H]⁺.

### Example 315. 5-((2-((3-(4-(8-Bromoquinoxalin-2-yl)-1H-pyrazol-1-yl)phenyl)amino)ethyl)amino)-2-(2,6-dioxopiperidin-3-yl)isoindoline-1,3-dione (GS-357)

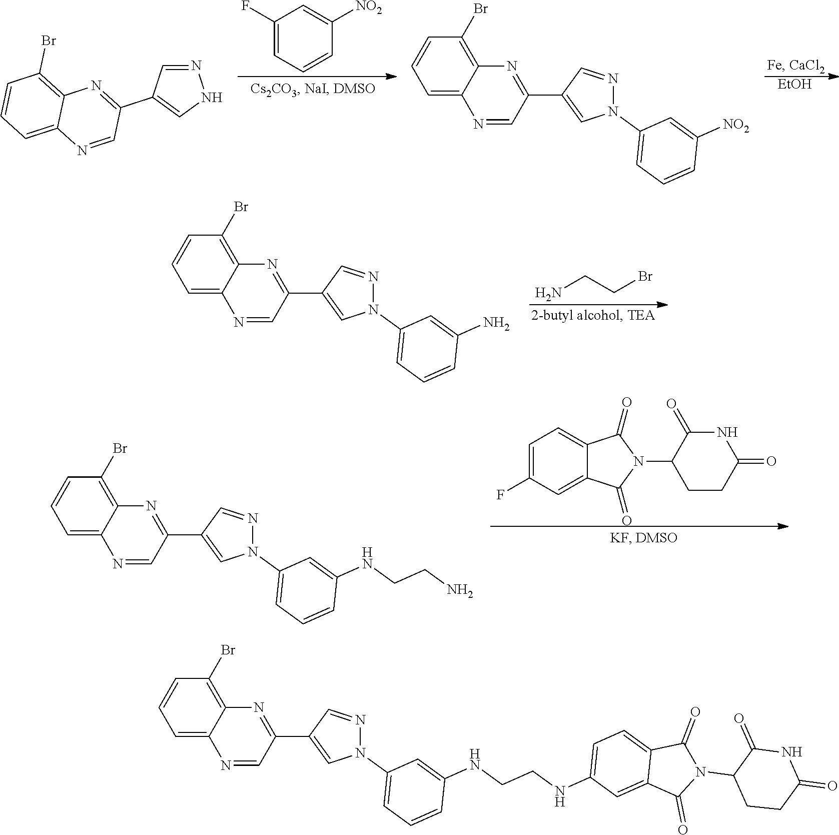

Step 1. Synthesis of 8-bromo-2-(1-(3-nitrophenyl)-1H-pyrazol-4-yl)quinoxaline

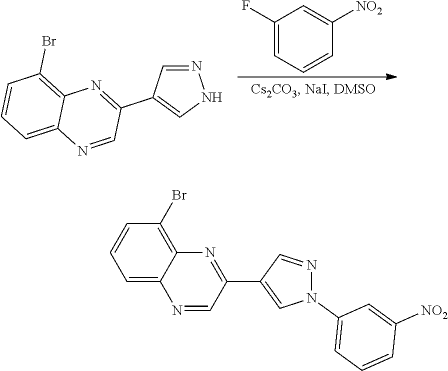 [SMILES: Brc1cccc2ncc(-c3cn[nH]c3)nc12 ; O=[N+]([O-])c1cccc(F)c1 ; O=[N+]([O-])c1cccc(-n2cc(-c3cnc4cccc(Br)c4n3)cn2)c1]

To a solution of 8-bromo-2-(1H-pyrazol-4-yl)quinoxalinee (137 mg, 0.50 mmol) and 1-fluoro-3-nitrobenzene (70 mg, 0.50 mmol) in DMSO (3 mL) were added Cs₂CO₃ (162 mg, 0.50 mmol) and NaI (30 mg, 0.20 mmol) at rt. Then the reaction mixture was stirred at 80° C. for 5 h. After cooling down to rt, the mixture was purified by reverse-phase chromatography to provide the desired product (100 mg, 49% yield) as a light-yellow solid. MS (ESI) m/z=396.0 [M+H]⁺.

Step 2. Synthesis of 3-(4-(8-bromoquinoxalin-2-yl)-1H-pyrazol-1-yl)aniline

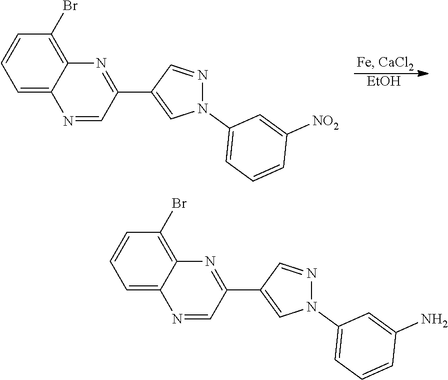

To a solution of 8-bromo-2-(1-(3-nitrophenyl)-1H-pyrazol-4-yl)quinoxaline (100 mg, 0.25 mmol) in ethanol (5 mL) were added iron powder (28 mg, 0.50 mmol) and CaCl₂ (28 mg, 0.25 mmol) at rt. Then the reaction mixture was stirred at 80° C. for 6 h. After cooling down to rt, the mixture was filtered through Celite. The filtrate was concentrated and purified by reverse-phase chromatography to provide the desired product (60 mg, 65% yield) as a light-yellow solid. MS (ESI) m/z=366.0 [M+H]⁺. The remaining steps were performed according to the standard procedures for preparing GS-351 to provide the desired product (9.5 mg, 55% yield) as a light-yellow solid. MS (ESI) m/z=665.2 [M+H]⁺.

### Example 316. 2-(2,6-Dioxopiperidin-3-yl)-5-(1-(3-(4-(quinoxalin-2-yl)-1H-pyrazol-1-yl)propyl)piperidin-4-yl)isoindoline-1,3-dione (GS-358)

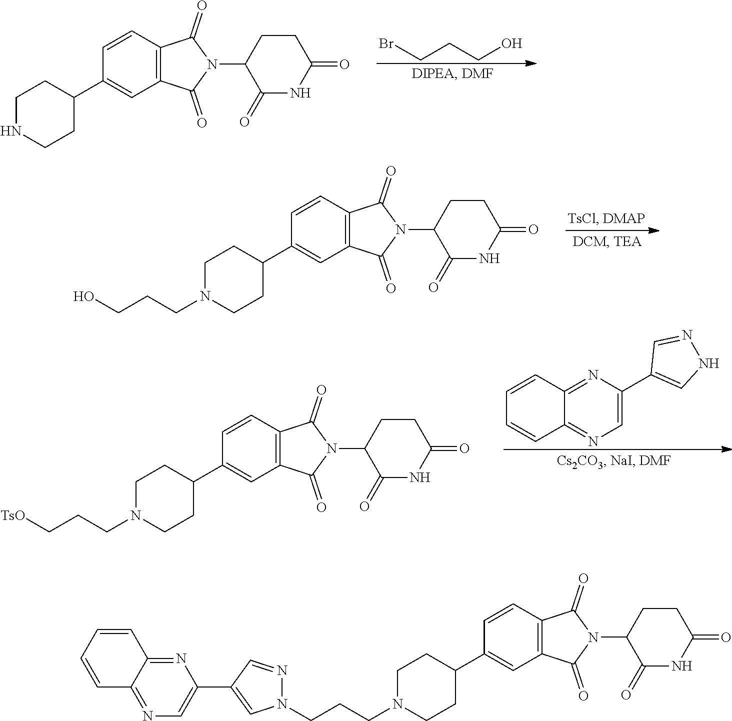 [SMILES: O=C1CCC(N2C(=O)c3ccc(C4CCNCC4)cc3C2=O)C(=O)N1 ; OCCCBr ; O=C1CCC(N2C(=O)c3ccc(C4CCN(CCCO)CC4)cc3C2=O)C(=O)N1 ; Cc1ccc(S(=O)(=O)OCCCN2CCC(c3ccc4c(c3)C(=O)N(C3CCC(=O)NC3=O)C4=O)CC2)cc1 ; c1ccc2nc(-c3cn[nH]c3)cnc2c1 ; O=C1CCC(N2C(=O)c3ccc(C4CCN(CCCn5cc(-c6cnc7ccccc7n6)cn5)CC4)cc3C2=O)C(=O)N1]

Step 1. Synthesis of 2-(2,6-dioxopiperidin-3-yl)-5-(piperidin-4-yl)isoindoline-1,3-dione

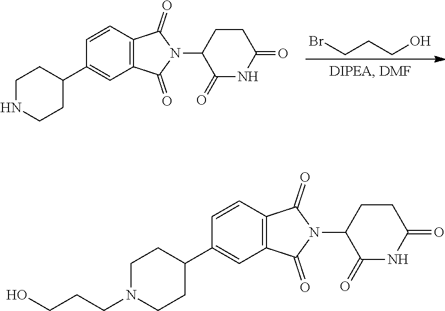

To a solution of 2-(2,6-dioxopiperidin-3-yl)-5-(piperidin-4-yl)isoindoline-1,3-dione (30 mg, 0.08 mmol) in DMF (3 mL) were added 3-bromopropan-1-ol (17 mg, 0.12 mmol) and DIPEA (31 mg, 0.24 mmol) at 0° C. The reaction mixture was stirred at 70° C. for 16 h. After cooling down to rt, the mixture was purified by reverse-phase chromatography to provide the desired product (25 mg, 78% yield) as a white solid. MS (ESI) m/z=400.3 [M+H]⁺.

Step 2. Synthesis of 3-(4-(2-(2,6-dioxopiperidin-3-yl)-1,3-dioxoisoindolin-5-yl)piperidin-1-yl)propyl 4-methylbenzenesulfonate

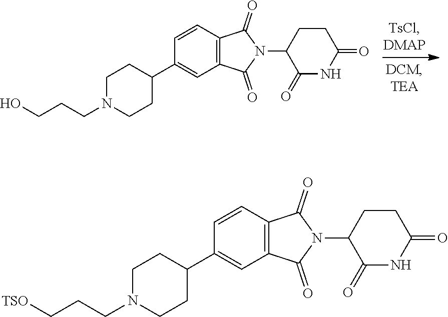

To a solution of 2-(2,6-dioxopiperidin-3-yl)-5-(1-(3-hydroxypropyl)piperidin-4-yl)isoindoline-1,3-dione (20 mg, 0.05 mmol) in DCM (5 mL) and TEA (2 mL) were added TsCl (15 mg, 0.08 mmol) and DMAP (6 mg, 0.05 mmol) at 0° C. The reaction mixture was stirred at rit for 1 h, before it was quenched with water and extracted with DCM (3×10 mL). The combined organic layers were washed with saturated brine, dried over anhydrous sodium sulfate, filtered, and concentrated under reduced pressure. The resulting residue was purified by prep-TLC to provide the desired product (12 mg, 45% yield) as a yellow solid. MS (ESI) m/z=554.2 [M+H]⁺.

Step 3. Synthesis of 2-(2,6-dioxopiperidin-3-yl)-5-(1-(3-(4-(quinoxalin-2-yl)-1H-pyrazol-1-yl)propyl)piperidin-4-yl)isoindoline-1,3-dione

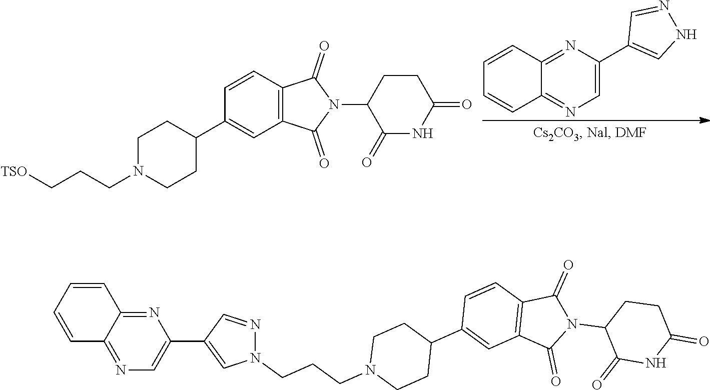

To a solution of 3-(4-(2-(2,6-dioxopiperidin-3-yl)-1,3-dioxoisoindolin-5-yl)piperidin-1-yl)propyl 4-methylbenzenesulfonate (12 mg, 0.02 mmol) in DMF (2 mL) were added 2-(1H-pyrazol-4-yl)quinoxaline (4 mg, 0.02 mmol), NaI (3 mg, 0.02 mmol) and Cs₂CO₃ (9.8 mg, 0.03 mmol) at rt. The reaction mixture was stirred at 100° C. for 3 h. After cooling down to rt, The reaction was purified by reverse-phase chromatography, followed by prep-TLC to provide the desired product (4.3 mg, 36% yield) as a white solid. MS (ESI) m/z=578.3 [M+H]⁺.

### Example 317. N-((2-(2,6-Dioxopiperidin-3-yl)-1,3-dioxoisoindolin-5-yl)methyl)-2-(3-(4-(pyridin-2-yl)piperazin-1-yl)piperidin-1-yl)acetamide (GS-359)

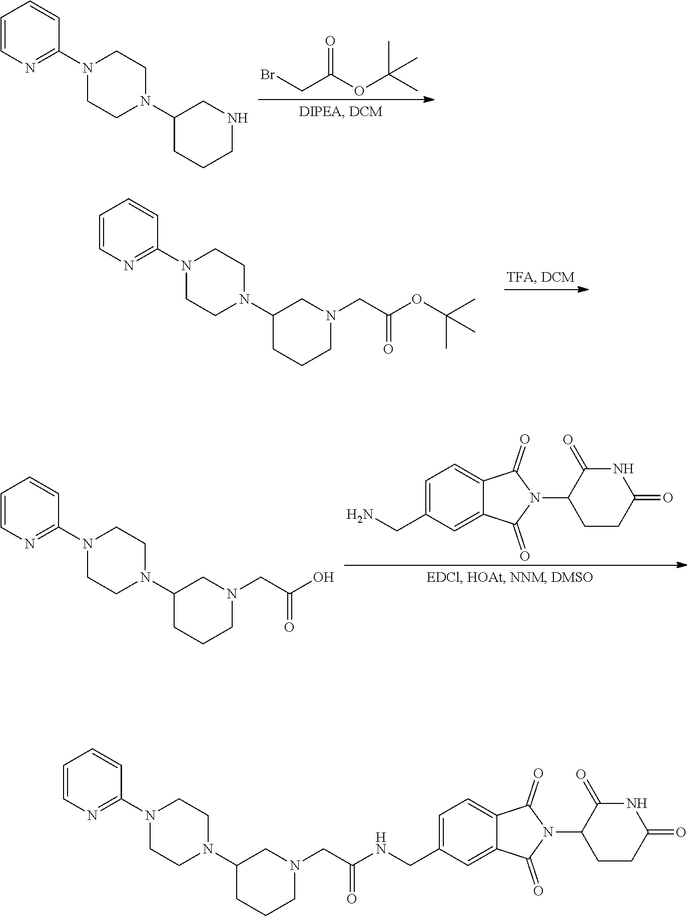

GS-359 was synthesized following the standard procedures for preparing GS-316 (2.2 mg, 12%/yield) as a light-yellow solid. MS (ESI) m/z=574.3 [M+H]⁺.

### Example 318. 2-(2,6-Dioxopiperidin-3-yl)-5-((4-(4-(quinoxalin-2-yl)-1H-pyrazol-1-yl)butyl)amino)isoindoline-1,3-dione (GS-360)

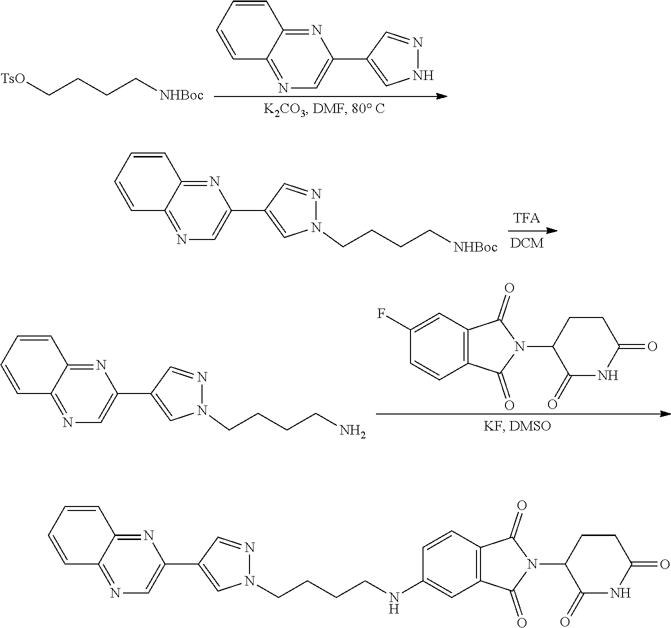 [SMILES: Cc1ccc(S(=O)(=O)OCCCCNC(=O)OC(C)(C)C)cc1 ; c1ccc2nc(-c3cn[nH]c3)cnc2c1 ; CC(C)(C)OC(=O)NCCCCn1cc(-c2cnc3ccccc3n2)cn1 ; NCCCCn1cc(-c2cnc3ccccc3n2)cn1 ; O=C1CCC(N2C(=O)c3ccc(F)cc3C2=O)C(=O)N1 ; O=C1CCC(N2C(=O)c3ccc(NCCCCn4cc(-c5cnc6ccccc6n5)cn4)cc3C2=O)C(=O)N1]

GS-360 was synthesized following the standard procedures for preparing GS-326 (2.2 mg, 12% yield) as a light-yellow solid. MS (ESI) m/z=524.3 [M+H]⁺.

### Example 319. 3-(1-Oxo-5-((6-(4-(quinoxalin-2-yl)-1H-pyrazol-1-yl)hexyl)amino)isoindolin-2-yl)piperidine-2,6-dione (GS-361)

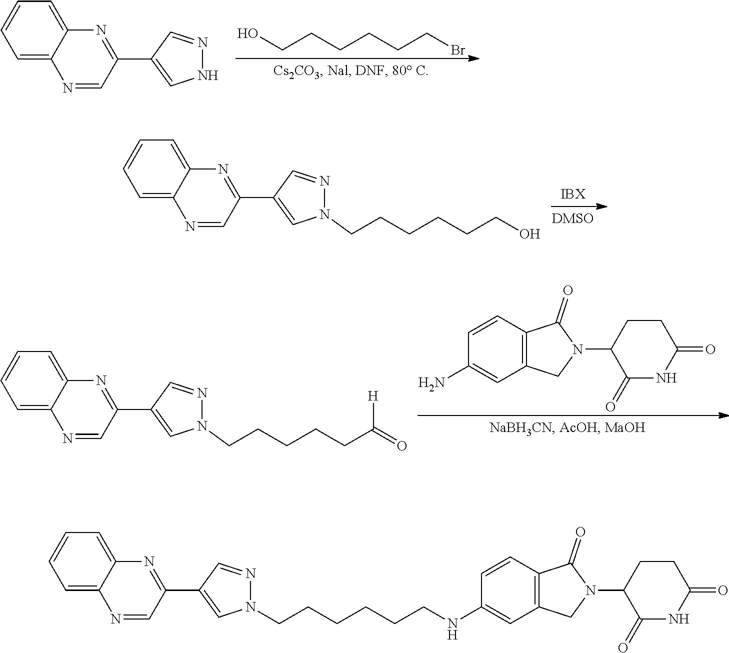 [SMILES: c1ccc2nc(-c3cn[nH]c3)cnc2c1 ; OCCCCCCBr ; OCCCCCCn1cc(-c2cnc3ccccc3n2)cn1 ; O=CCCCCCn1cc(-c2cnc3ccccc3n2)cn1 ; Nc1ccc2c(c1)CN(C1CCC(=O)NC1=O)C2=O ; O=C1CCC(N2Cc3cc(NCCCCCCn4cc(-c5cnc6ccccc6n5)cn4)ccc3C2=O)C(=O)N1]

Step 1. Synthesis of 6-(4-(quinoxalin-2-yl)-1H-pyrazol-1-yl)hexan-1-ol

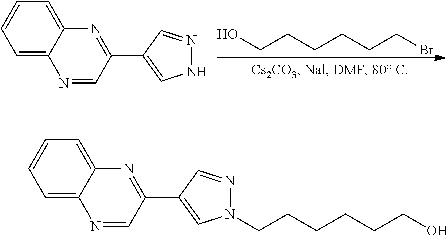

A mixture of 2-(1H-pyrazol-4-yl)quinoxaline (39 mg, 0.20 mmol), 6-bromohexan-1-ol (36 mg, 0.20 mmol), Cs₂CO₃ (130 mg, 0.40 mmol) and NaI (31 mg, 0.20 mmol) in DMF (4 mL) was stirred at 80° C. for 2 h. The reaction mixture was poured into water (10 mL) and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (46 mg, 78% yield) as a light-yellow solid. MS (ESI) m/z=297.2 [M+H]⁺.

Step 2. Synthesis of 6-(4-(quinoxalin-2-yl)-1H-pyrazol-1-yl)hexanal

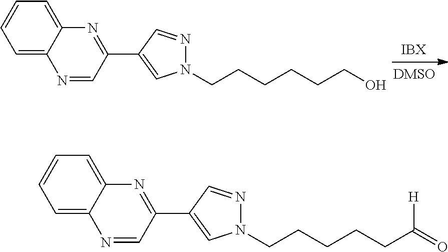 [SMILES: OCCCCCCn1cc(-c2cnc3ccccc3n2)cn1 ; O=CCCCCCn1cc(-c2cnc3ccccc3n2)cn1]

To a solution of 6-(4-(quinoxalin-2-yl)-1H-pyrazol-1-yl)hexan-1-ol (30 mg, 0.10 mmol) in DMSO (3 mL) was added IBX (33 mg, 0.12 mmol) at rt. The reaction was stirred at rt for 8 h. Then the mixture was purified by reverse-phase chromatography to provide the desired product (14 mg, 48% yield) as a light-yellow solid. MS (ESI) m/z=295.1 [M+H]⁺.

Step 3. Synthesis of 3-(1-oxo-5-((6-(4-(quinoxalin-2-yl)-1H-pyrazol-1-yl)hexyl)amino) isoindolin-2-yl)piperidine-2,6-dione

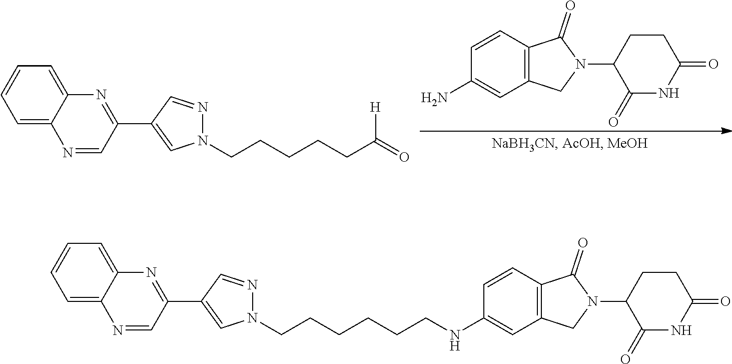 [SMILES: O=CCCCCCn1cc(-c2cnc3ccccc3n2)cn1 ; Nc1ccc2c(c1)CN(C1CCC(=O)NC1=O)C2=O ; O=C1CCC(N2Cc3cc(NCCCCCCn4cc(-c5cnc6ccccc6n5)cn4)ccc3C2=O)C(=O)N1]

A mixture of 6-(4-(quinoxalin-2-yl)-1H-pyrazol-1-yl)hexanal (8 mg, 0.03 mmol), 3-(5-amino-1-oxoisoindolin-2-yl)piperidine-2,6-dione (7.8 mg, 0.03 mmol), NaBH₃CN (5.7 mg, 0.09 mmol) and AcOH (1 drop) in MeOH (3 mL) was stirred at 50° C. for 3 h. The solution was poured into water (10 mL) and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (4.3 mg, 29% yield) as a light-yellow solid. MS (ESI) mnz 538.2 [M+H]⁺.

### Example 320. 5-((2-((3-(4-(4-(8-Bromoquinoxalin-2-yl)-1H-pyrazol-1-yl)piperidin-1-yl)phenyl)amino)ethyl)amino)-2-(2,6-dioxopiperidin-3-yl)isoindoline-1,3-dione (GS-362)

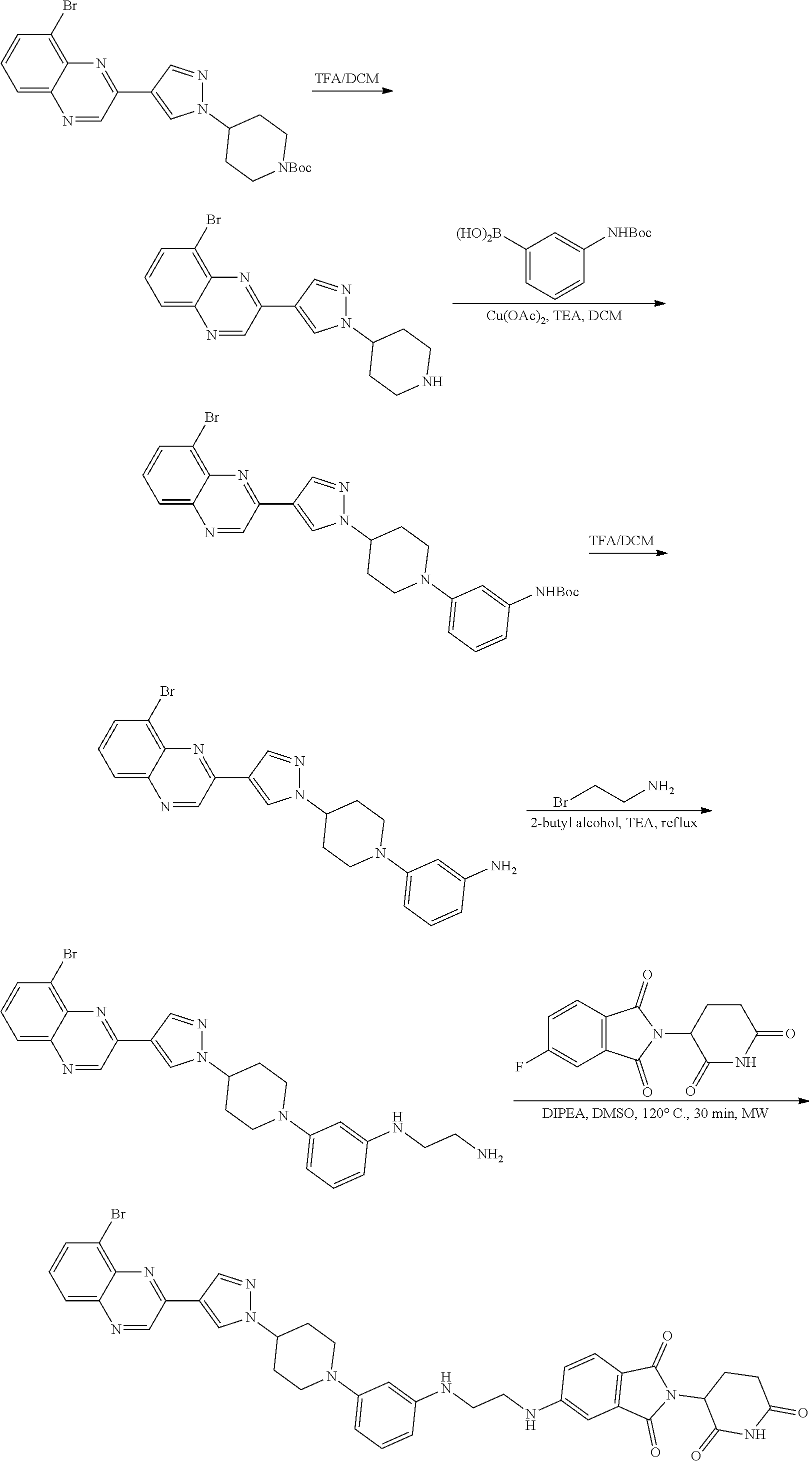

GS-362 was synthesized following the standard procedures for preparing GS-351 (4.6 mg, 31% yield) as a light-yellow solid. MS (ESI) m/z=748.2 [M+H]⁺.

### Example 321. 2-(2,6-Dioxopiperidin-3-yl)-5-(4-(3-(4-(quinoxalin-2-yl)-1H-pyrazol-1-yl)propyl)piperidin-1-yl)isoindoline-1,3-dione (GS-363)

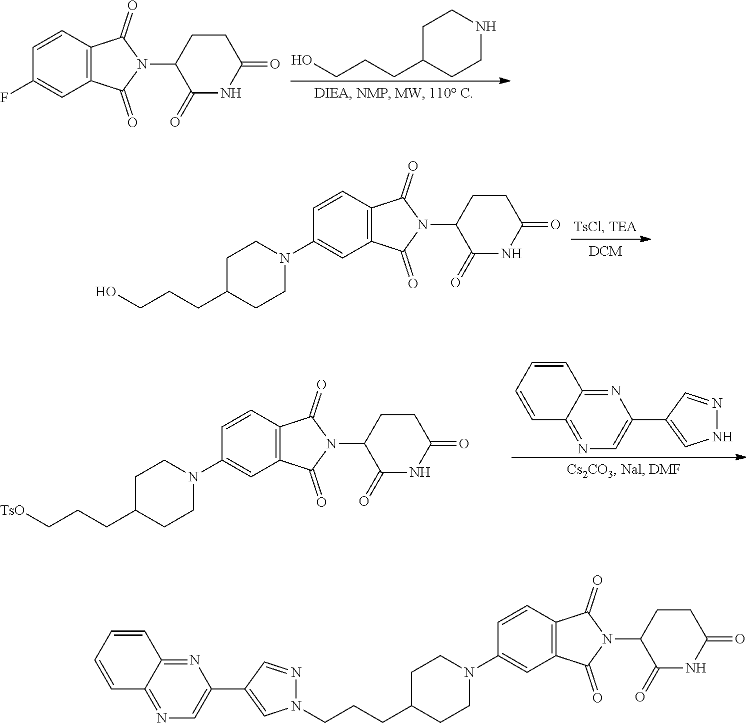

Step 1. Synthesis of 2-(2,6-dioxopiperidin-3-yl)-5-(4-(3-hydroxypropyl)piperidin-1-yl)isoindoline-1,3-dione

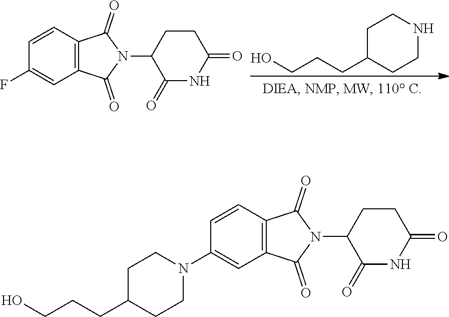

To a solution of 2-(2,6-dioxopiperidin-3-yl)-5-fluoroisoindoline-1,3-dione (100 mg, 0.36 mmol) in NMP (2 mL) were added 3-(piperidin-4-yl)propan-1-ol (56 mg, 0.39 mmol) and DIEA (139 mg, 1.08 mmol). The reaction mixture was stirred at 110° C. for 45 min under microwave irradiation. The reaction was quenched with water (10 mL) and extracted with ethyl acetate (3×10 mL). The combined organic phase was dried over anhydrous sodium sulfate, filtered, and concentrated under reduced pressure. The resulting residue was purified by silica gel flash chromatography (petroleum ether/ethyl acetate=1:1) to provide the desired product (120 mg, 82%) as a yellow oil. MS (ESI) m/z=400.2 [M+H]⁺.

Step 2. Synthesis of 3-(1-(2-(2,6-dioxopiperidin-3-yl)-1,3-dioxoisoindolin-5-yl)piperidin-4-yl)propyl 4-methylbenzenesulfonate

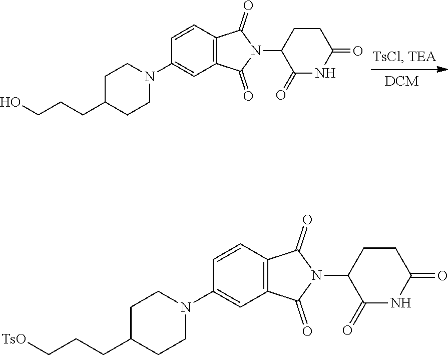 [SMILES: O=C1CCC(N2C(=O)c3ccc(N4CCC(CCCO)CC4)cc3C2=O)C(=O)N1 ; Cc1ccc(S(=O)(=O)OCCCC2CCN(c3ccc4c(c3)C(=O)N(C3CCC(=O)NC3=O)C4=O)CC2)cc1]

To a solution of 2-(2,6-dioxopiperidin-3-yl)-5-(4-(3-hydroxypropyl)piperidin-1-yl)isoindoline-1,3-dione (50 mg, 0.125 mmol) in DCM (5 mL) were added TEA (38 mg, 0.375 mmol) and TsCl (35 mg, 0.19 mmol) at 0° C. The reaction mixture was stirred at rt for 2 h, before it was concentrated and purified by silica gel flash chromatography (petroleum ether/ethyl acetate=5:1) to provide the desired product (53 mg, 76%) as a yellow oil. MS (ESI) m/z=554.2 [M+H]⁺.

Step 3. Synthesis of 2-(2,6-dioxopiperidin-3-yl)-5-(4-(3-(4-(quinoxalin-2-yl)-1H-pyrazol-1-yl)propyl)piperidin-1-yl)isoindoline-1,3-dione

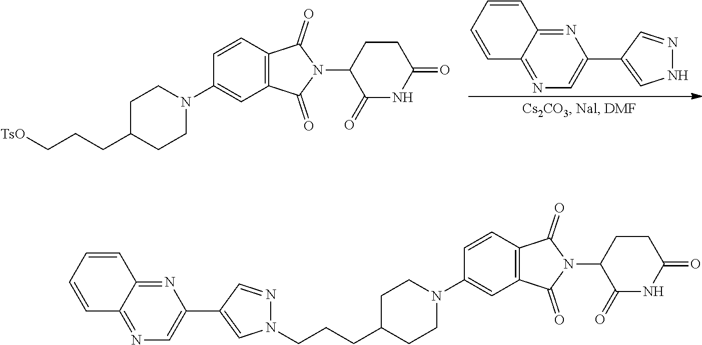

To a solution of 3-(1-(2-(2,6-dioxopiperidin-3-yl)-1,3-dioxoisoindolin-5-yl)piperidin-4-yl)propyl 4-methylbenzenesulfonate (53 mg, 0.09 mmol) in DMF (1 mL) were added Cs₂CO₃ (65 mg, 0.2 mmol) and NaI (29 mg, 0.09 mmol). The reaction mixture was stirred at 80° C. for 1 h. After cooling down to rt the reaction mixture was purified by reverse phase to provide the desired product (3 mg, 6%) as a yellow solid. MS (ESI) m/z=578.3 [M+H]⁺.

### Example 322. 2-(2,6-Dioxopiperidin-3-yl)-5-(1-(4-(4-(quinoxalin-2-yl)-1H-pyrazol-1-yl)butyl)azetidin-3-yl)isoindoline-1,3-dione (GS-364)

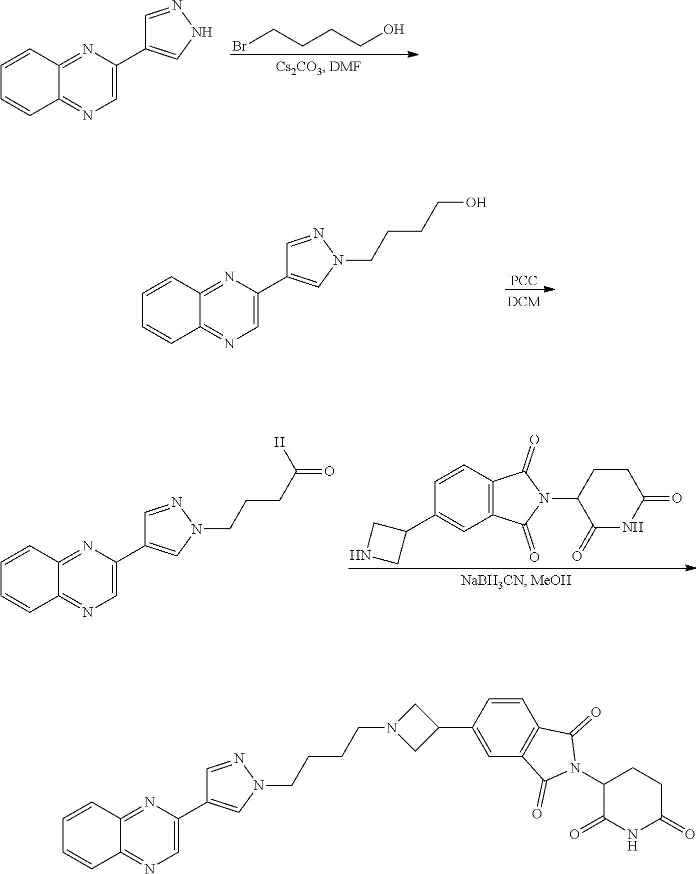

Step 1. Synthesis of 4-(4-(quinoxalin-2-yl)-1H-pyrazol-1-yl)butan 1-ol

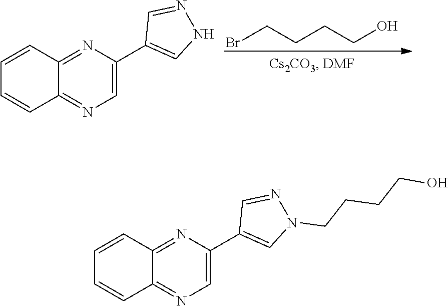

To a solution of 2-(1H-pyrazol-4-yl)quinoxaline (60 mg, 0.3 mmol) in DMF (5 mL) were added 4-bromobutan-1-ol (92 mg, 0.6 mmol) and Cs₂CO₃ (293 mg, 0.9 mmol). The reaction mixture was stirred at 90° C. for 16 h, before it was purified by reverse-phase chromatography to provide the desired product (28 mg, 35% yield) as a white solid. MS (ESI) m/z=269.2 [M+H]⁺.

Step 2. Synthesis of 4-(4-(quinoxalin-2-yl)-1H-pyrazol-1-yl)butanal

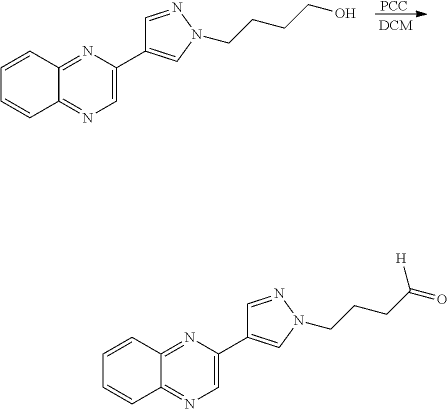

To a solution of 4-(4-(quinoxalin-2-yl)-1H-pyrazol-1-yl)butan-1-ol (10 mg, 0.04 mmol) in DCM (3 mL) was added PCC (16 mg, 0.08 mmol) at 0° C. Then the reaction mixture was stirred at rt for 1 h. The reaction was quenched with water and extracted with DCM (3×10 mL). The combined organic layers were washed with saturated brine, dried over anhydrous sodium sulfate, filtered, and concentrated under reduced pressure. The resulting residue was purified by column chromatography to provide the desired product (4.0 mg, 35% yield) as a brown solid. MS (ESI) m/z=267.2 [M+H]⁺.

Step 3. Synthesis of 2-(2,6-dioxopiperidin-3-yl)-5-(1-(4-(4-(quinoxalin-2-yl)-1H-pyrazol-1-yl)butyl)azetidin-3-yl)isoindoline-1,3-dione

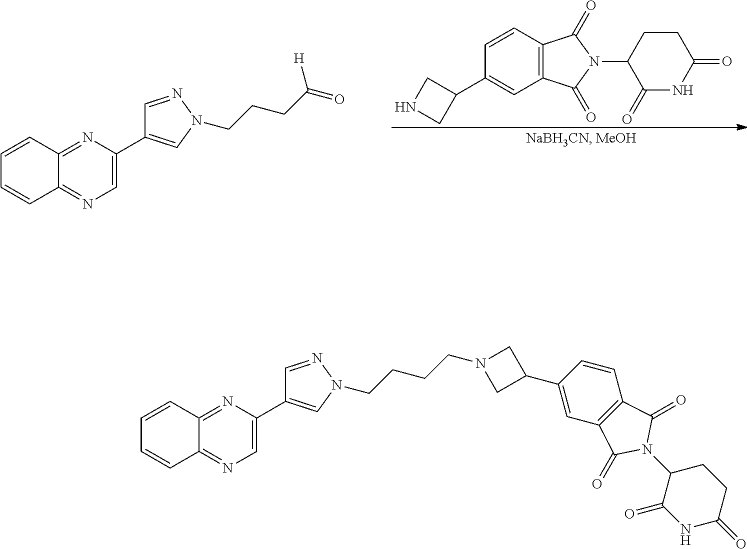 [SMILES: O=CCCCn1cc(-c2cnc3ccccc3n2)cn1 ; O=C1CCC(N2C(=O)c3ccc(C4CNC4)cc3C2=O)C(=O)N1 ; O=C1CCC(N2C(=O)c3ccc(C4CN(CCCCn5cc(-c6cnc7ccccc7n6)cn5)C4)cc3C2=O)C(=O)N1]

To a solution of 4-(4-(quinoxalin-2-yl)-1H-pyrazol-1-yl)butanal (4 mg, 0.015 mmol) in MeOH (2 mL) were added 5-(azetidin-3-yl)-2-(2,6-dioxopiperidin-3-yl)isoindoline-1,3-dione (4.7 mg, 0.015 mmol) and NaBH₃CN (3 mg, 0.04 mmol) at 0° C. The reaction was stirred at rt for 16 h. Then the mixture was purified by reverse-phase chromatography, followed by prep-TLC to provide the desired product (4.3 mg, 51% yield) as a yellow solid. MS (ESI) m/z=564.4 [M+H]⁺.

### Example 323. 2-(2,6-Ddioxopiperidin-3-yl)-5-((3-(3-(4-(quinoxalin-2-yl)-1H-pyrazol-1-yl)phenyl)propyl) amino)isoindoline-1,3-dione (GS-365)

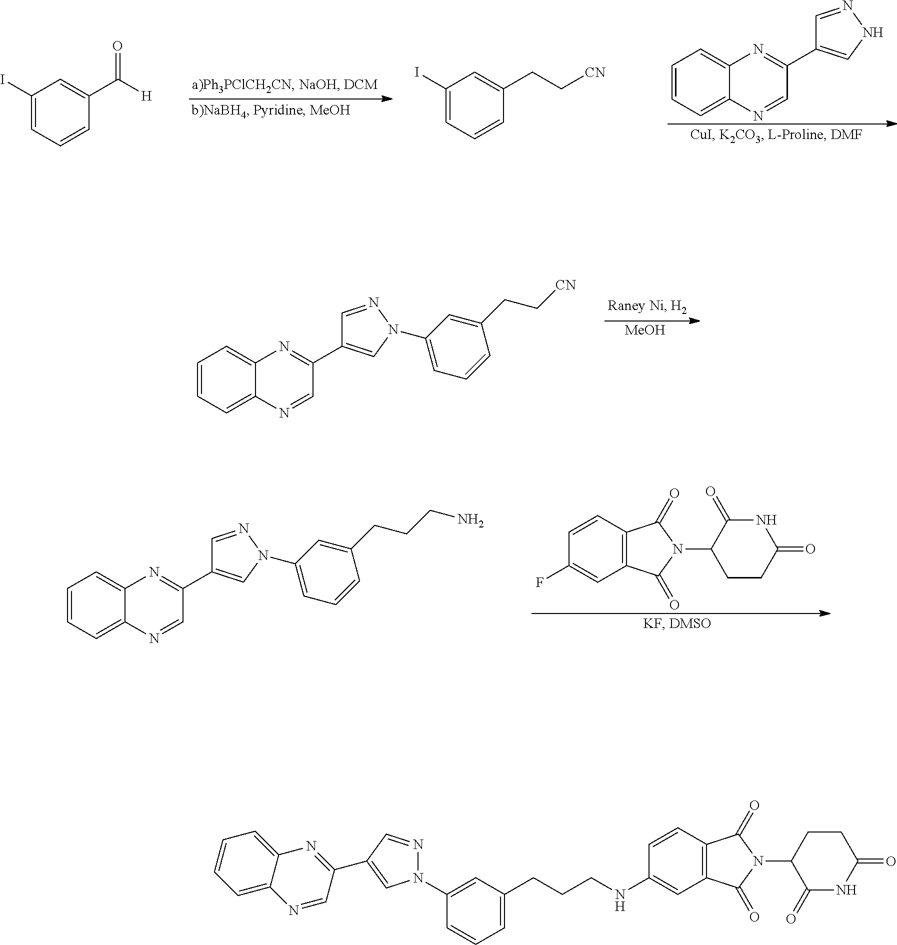

Step 1. Synthesis of 3-(3-iodophenyl)propanenitrile

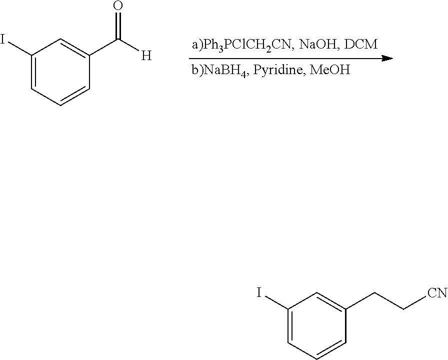 [SMILES: O=Cc1cccc(I)c1 ; N#CCCc1cccc(I)c1]

To a solution of 3-iodobenzaldehyde (1.3 g, 5.62 mmol) in DCM (50 mL) were added (cyanomethyl)triphenylphos-phonium chloride (3.04 g, 9.01 mmol) and 30% aqueous sodium hydroxide solution (50 mL). The reaction mixture was stirred at rt for 30 min, before it was poured into water (50 mL) and extracted with DCM (3×50 mL). The combined organic layers were washed with saturated brine (20 mL), dried over anhydrous sodium sulfate, filtered, and concentrated under reduced pressure. The resulting residue was dissolved in pyridine (15 mL) and methanol (5 mL), then NaBH₄ (278 mg, 7.3 mmol) was added under N₂. The mixture was refluxed for 3 h, before the reaction was concentrated under reduced pressure. The resulting residue was neutralized with cold HCl (6N) and extracted with ethyl acetate (3×30 mL). The combined organic layers were washed with saturated brine (50 mL), dried over anhydrous sodium sulfate, filtered, and concentrated under reduced pressure. The resulting residue was purified by silica gel column chromatography to provide the desired product (800 mg, 55% yield) as a colorless oil. MS (ESI) m/z=258.1 [M+H]⁺

Step 2. Synthesis of 3-(3-(4-(quinoxalin-2-yl)-1H-pyrazol-1-yl)phenyl)propanenitrile

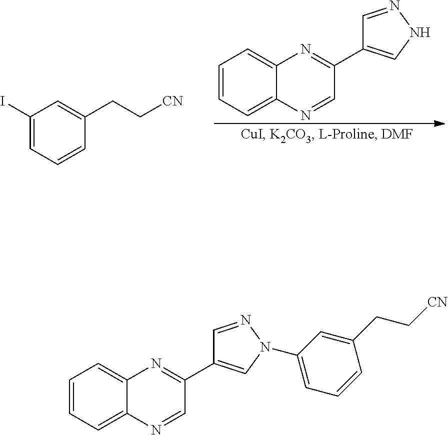 [SMILES: N#CCCc1cccc(I)c1 ; c1ccc2nc(-c3cn[nH]c3)cnc2c1 ; N#CCCc1cccc(-n2cc(-c3cnc4ccccc4n3)cn2)c1]

To a solution of 3-(3-iodophenyl)propanenitrile (100 mg, 0.39 mmol) in DMF (10 mL) were added 2-(1H-pyrazol-4-yl)quinoxaline (76 mg, 0.39 mmol), Cs₂CO₃ (382 mg, 1.17 mmol), L-proline (14 mg, 0.12 mmol) and CuI (8 mg, 0.04 mmol) at rt under N₂. The reaction mixture was heated at 90° C. for 5 h, before it was concentrated and purified by reverse-phase chromatography to provide the desired product (50 mg, 39% yield) as a brown solid. MS (ESI) m/z=326.2 [M+H]⁺.

Step 3. Synthesis of 3-(3-(4-(quinoxalin-2-yl)-1H-pyrazol-1-yl)phenyl)propan-1-amine

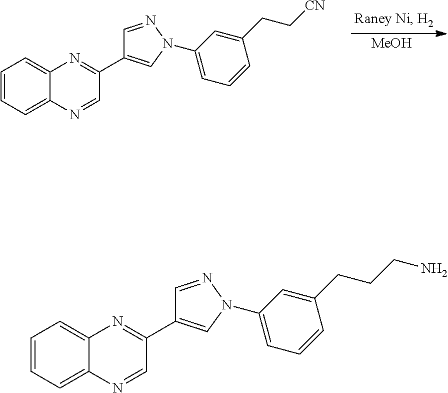 [SMILES: "MeOH" ; N#CCCc1cccc(-n2cc(-c3cnc4ccccc4n3)cn2)c1 ; NCCCc1cccc(-n2cc(-c3cnc4ccccc4n3)cn2)c1]

A mixture of 3-(3-(4-(quinoxalin-2-yl)-1H-pyrazol-1-yl)phenyl)propanenitrile (45 mg, 0.14 mmol) and Raney Ni (10 mg) in MeOH (5 mL) was stirred at rt for 4 h under hydrogen balloon. The solution was filtered through Celite and the filtrate was evaporated under reduced pressure. The resulting residue was purified by reverse-phase chromatography to provide the desired product (20 mg, 44% yield) as a brown solid. MS (ESI) m/z=330.2 [M+H]⁺.

Step 4. Synthesis of 2-(2,6-dioxopiperidin-3-yl)-5-((3-(3-(4-(quinoxalin-2-yl)-1H-pyrazol-1-yl)phenyl) propyl)amino)isoindoline-1,3-dione

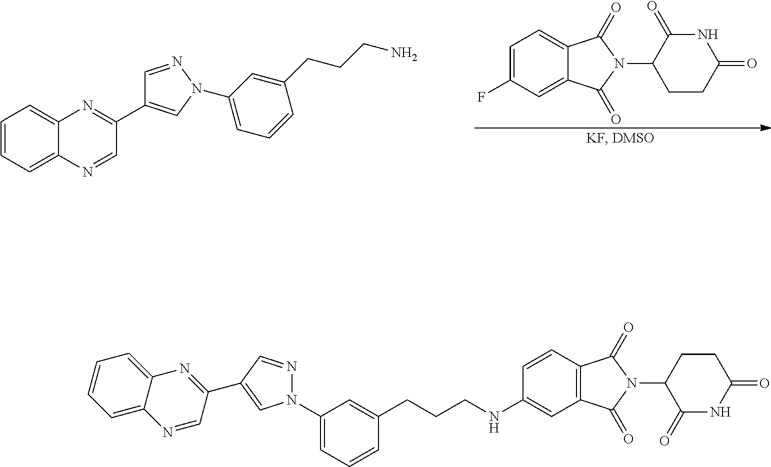 [SMILES: NCCCc1cccc(-n2cc(-c3cnc4ccccc4n3)cn2)c1 ; O=C1CCC(N2C(=O)c3ccc(F)cc3C2=O)C(=O)N1 ; O=C1CCC(N2C(=O)c3ccc(NCCCc4cccc(-n5cc(-c6cnc7ccccc7n6)cn5)c4)cc3C2=O)C(=O)N1]

To a solution of 3-(3-(4-(quinoxalin-2-yl)-1H-pyrazol-1-yl)phenyl)propan-1-amine (20 mg, 0.06 mmol) in DMSO (2 mL) were added 2-(2,6-dioxopiperidin-3-yl)-5-fluoroisoindoline-1,3-dione (25 mg, 0.09 mmol) and KF (18 mg, 0.3 mmol). The mixture was stirred at 130° C. under microwave irradiation for 0.5 h, before it was purified by reverse-phase chromatography to provide the desired product (4 mg, 11% yield) as a yellow solid. MS (ESI) m/z=586.4 [M+H]⁺.

### Example 324. 2-(2,6-Dioxopiperidin-3-yl)-5-((2-((3-(4-phenylpiperidin-1-yl)phenyl)amino)ethyl)amino)isoindoline-1,3-dione (GS-366)

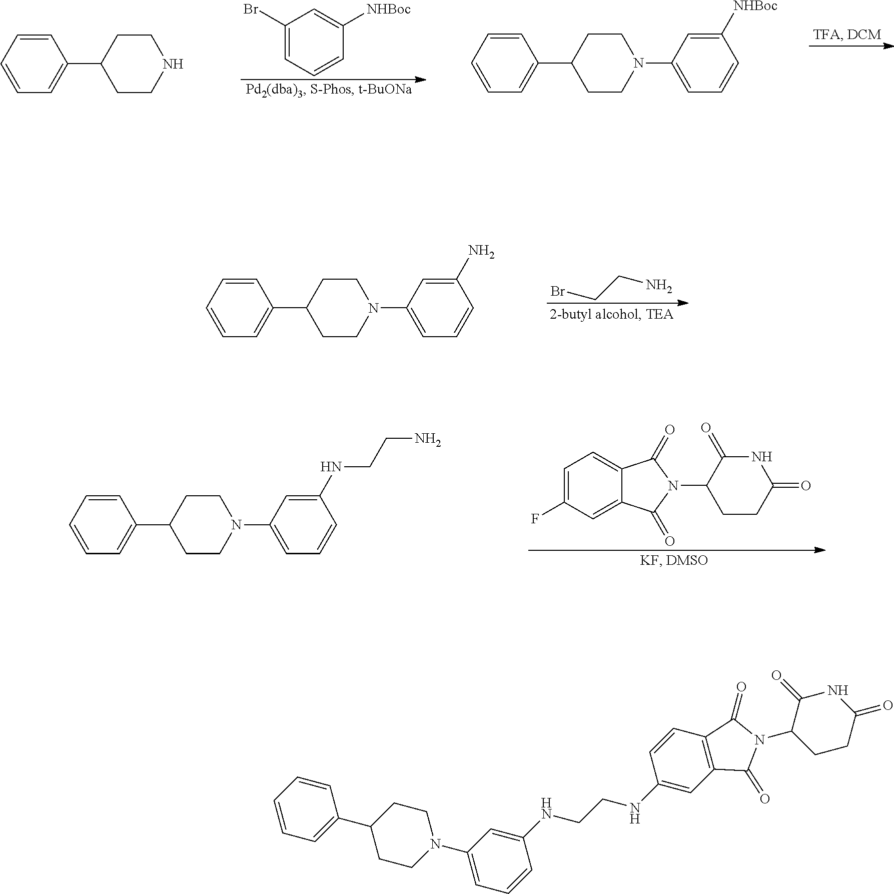

GS-366 was synthesized following the standard procedures for preparing GS-351 (6.8 mg, 42% yield) as a light-yellow solid. MS (ESI) m/z=552.3 [M+H]⁺.

### Example 325. 2-(2,6-Dioxopiperidin-3-yl)-5-(3-(4-(4-(quinoxalin-2-yl)-1H-pyrazol-1-yl)butyl)azetidin-1-yl)isoindoline-1,3-dione (GS-367)

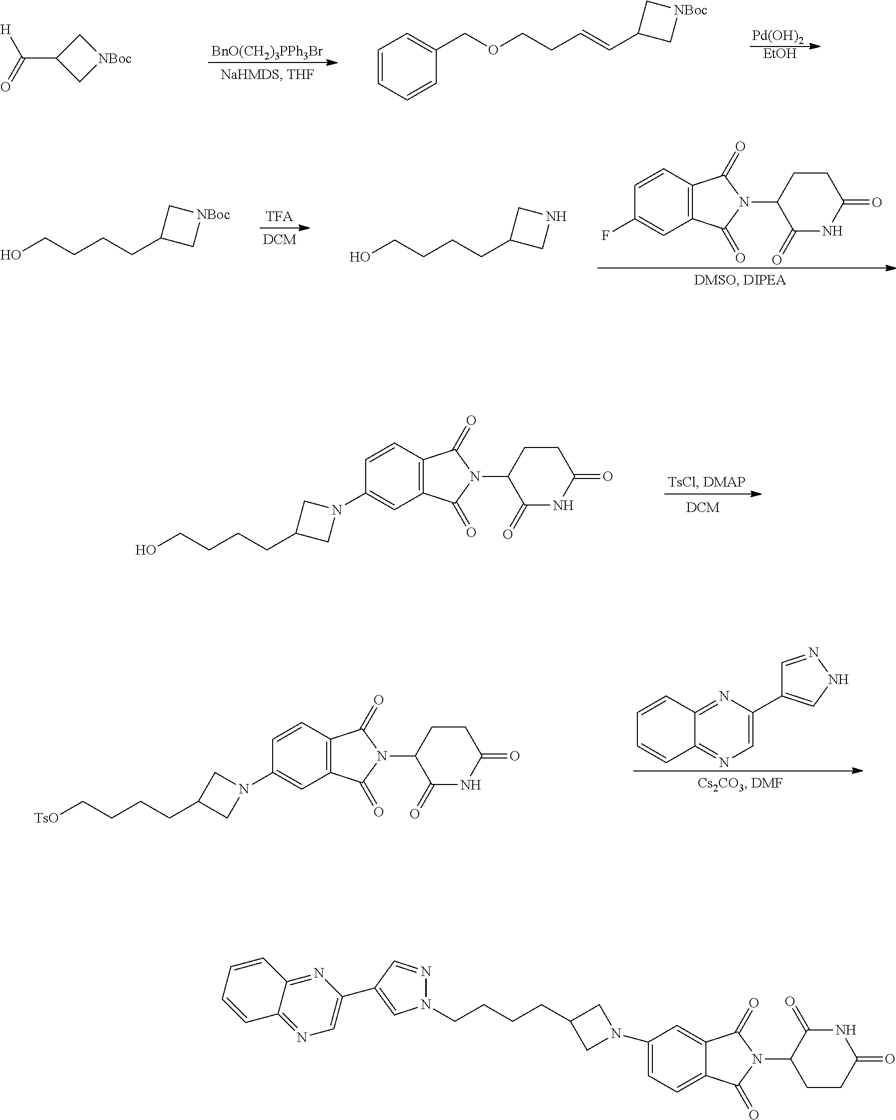

Step 1. Synthesis of tert-butyl (E)-3-(4-(benzyloxy)but-1-en-1-yl)azetidine-1-carboxylate

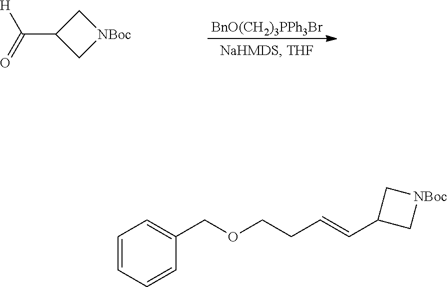 [SMILES: "BnO(CH2)3PPh3Br" ; CC(C)(C)OC(=O)N1CC(C=O)C1 ; CC(C)(C)OC(=O)N1CC(/C=C/CCOCc2ccccc2)C1]

To a solution of (3-(benzyloxy)propyl)bromotriphenyl-15-phosphane (212 mg, 0.44 mmol) in THF (10 mL) was added NaHMDS (2.0 M in THF, 0.22 mL, 0.44 mmol) at 0° C. under nitrogen atmosphere. After stirring at 0° C. for 0.5 h, tert-butyl 3-formylazetidine-1-carboxylate (40 mg, 0.22 mmol) in THF (5 mL) was added dropwise at 0° C. The resulting mixture was stirred at rt for 2 h, before it was quenched with water slowly and extracted with ethyl acetate (3×20 mL). The combined organic phase was washed with brine and dried over anhydrous Na₂SO₄, filtered, and concentrated. The resulting residue was purified by column chromatography on silico gel to provide the desired product (32 mg, 47% yield) as a colorless oil. MS (ESI) m/z=318.2 [M+H]⁺.

Step 2. Synthesis of tert-butyl 3-(4-hydroxybutyl)azetidine-1-carboxylate

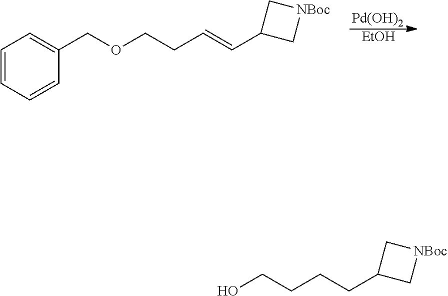

To a solution of tert-butyl (E)-3-(4-(benzyloxy)but-1-en-1-yl)azetidine-1-carboxylate (20 mg, 0.06 mmol) in EtOH (5 mL) was added 10% Pd(OH)₂ (5 mg). The mixture was purged with hydrogen and stirred at rt for 16 h under hydrogen balloon. The solution was filtered through Celite and the filtrate was concentrated to provide the desired product (13 mg, 94% yield) as a white solid. MS (ESI) m/z=230.2 [M+H]⁺.

Step 3. Synthesis of 4-(azetidin-3-yl)butan-1-ol

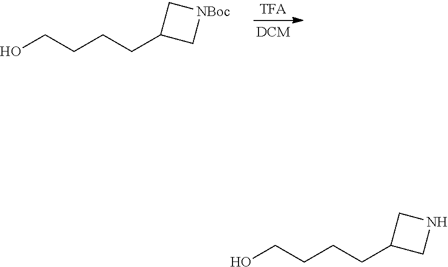 [SMILES: CC(C)(C)OC(=O)N1CC(CCCCO)C1 ; OCCCCC1CNC1]

To a solution of tert-butyl 3-(4-hydroxybutyl)azetidine-1-carboxylate (13 mg, 0.1 mmol) in DCM (2 mL) was added TFA (0.5 mL) at 0° C. After stirring at rt for 1 h, the solution was concentrated under vacuum. The resulting residue was used in the next step directly without further purification.

Step 4. Synthesis of 2-(2,6-dioxopiperidin-3-yl)-5-(3-(4-hydroxybutyl)azetidin-1-yl)isoindoline-1,3-dione

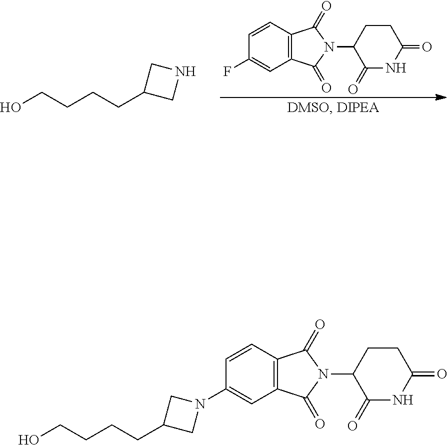 [SMILES: OCCCCC1CNC1 ; O=C1CCC(N2C(=O)c3ccc(F)cc3C2=O)C(=O)N1 ; O=C1CCC(N2C(=O)c3ccc(N4CC(CCCCO)C4)cc3C2=O)C(=O)N1]

To a solution of 4-(azetidin-3-yl)butan-1-ol (13 mg, 0.1 mmol) in DMSO (2 mL) were added 2-(2,6-dioxopiperidin-3-yl)-5-fluoroisoindoline-1,3-dione (27 mg, 0.1 mmol) and DIPEA (40 mg, 0.3 mmol). The reaction mixture was stirred at 90° C. for 1 h, before it was purified by reverse-phase chromatography to provide the desired product (11 mg, 29% yield) as a white solid. MS (ESI) m/z=386.2 [M+H]⁺.

The remaining steps were performed according to the procedures for preparing GS-358 to provide the desired product (3.4 mg, 31% yield) as a yellow solid. MS (ESI) m/z=564.4 [M+H]⁺.

### Example 326. 2-(2,6-Dioxopiperidin-3-yl)-5-((3-(3-(4-(4-(quinoxalin-2-yl)-1H-pyrazol-1-yl)piperidin-1-yl)phenyl)propyl)amino)isoindoline-1,3-dione (GS-368)

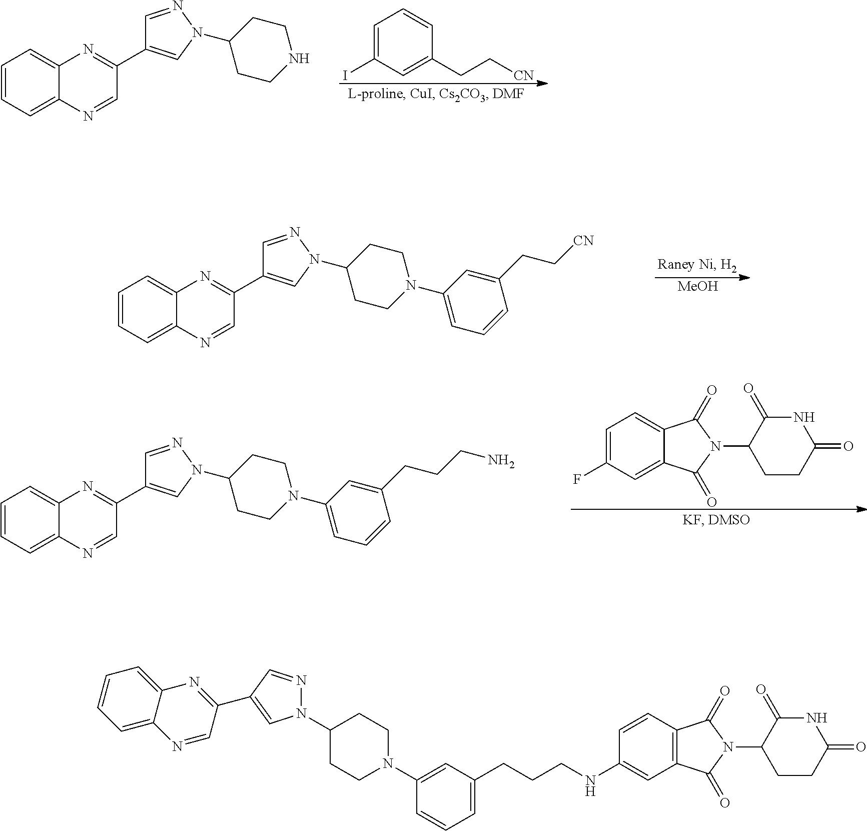

GS-368 was synthesized following the standard procedures for preparing GS-365 (3 mg, 12% yield) as a yellow solid. MS (ESI) m/z=669.3 [M+H]⁺.

### Example 327. 2-(2,6-Dioxopiperidin-3-yl)-5-(6-(4-(quinoxalin-2-yl)-1H-pyrazol-1-yl)hex-1-yn-1-yl)isoindoline-1,3-dione (GS-369)

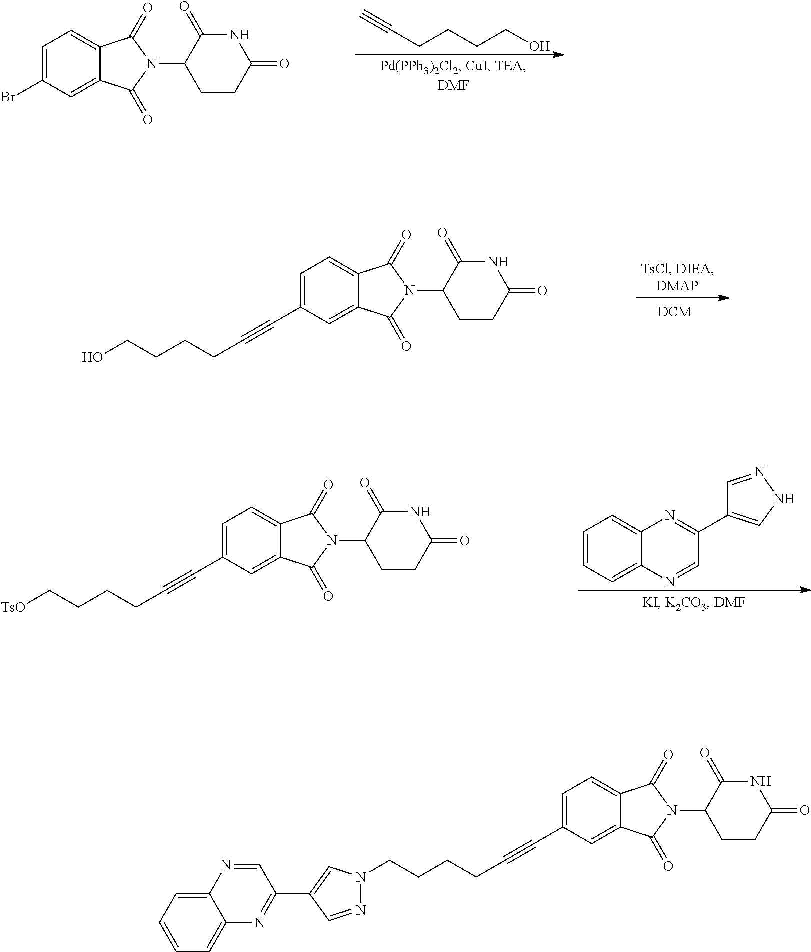

Step 1. Synthesis of 2-(2,6-dioxopiperidin-3-yl)-5-(6-hydroxyhex-1-yn-1-yl)isoindoline-1,3-dione

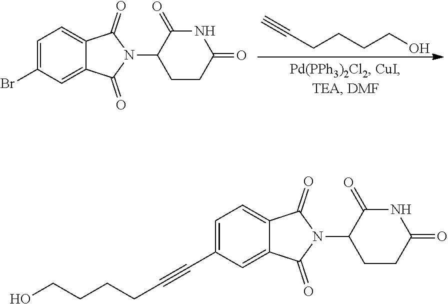

To a solution of 5-bromo-2-(2,6-dioxopiperidin-3-yl)isoindoline-1,3-dione (100 mg, 0.29 mmol) and hex-5-yn-1-ol (58 mg, 0.58 mmol) in DMF (3 mL) were added TEA (147 mg, 1.45 mmol), Pd(PPh₃)₂Cl₂ (21 mg, 0.03 mmol) and CuI (6 mg, 0.03 mmol) at rt. Then the reaction mixture was stirred at 80° C. under N₂ for 5 h. After cooling down to rt, the solution was concentrated and purified by reverse-phase chromatography to provide the desired product (90 mg, 85% yield) as a brown solid. MS (ESI) m/z=355.2 [M+H]⁺.

The remaining steps were performed according to the standard procedures for preparing GS-367 to provide the desired product (5 mg, 10% yield) as a white solid. MS (ESI) m/z=533.2 [M+H]⁺.

### Example 328. 2-(2,6-Dioxopiperidin-3-yl)-5-((2-((3-(4-(thiazol-2-yl)piperidin-1-yl)phenyl)amino)ethyl)amino)isoindoline-1,3-dione (GS-370)

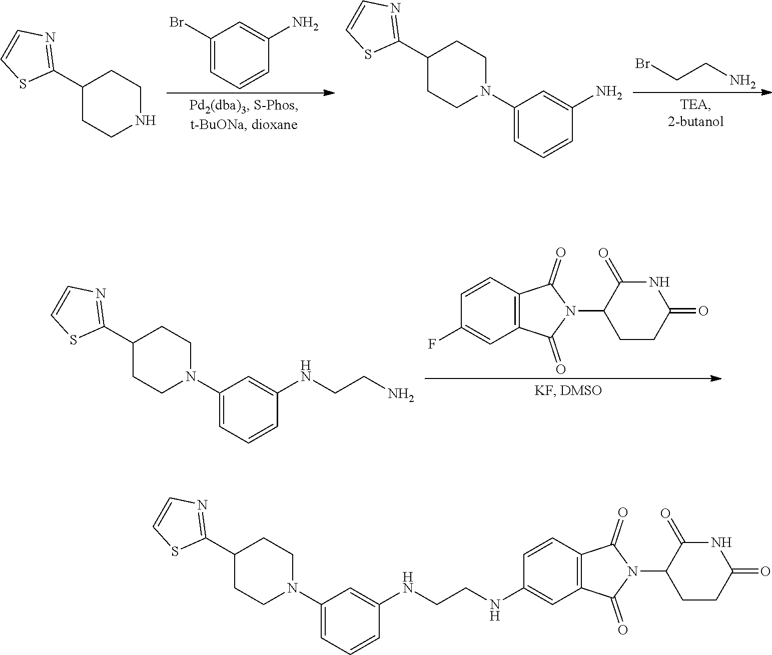

GS-370 was synthesized following the standard procedures for preparing GS-351 (3.2 mg, 10% yield) as a light-yellow solid. MS (ESI) m/z=559.4 [M+H]⁺.

### Example 329. 2-(2,6-Dioxopiperidin-3-yl)-5-((3-(3-(4-(quinoxalin-2-yl)-1H-pyrazol-1-yl)azetidin-1-yl)propyl)amino)isoindoline-1,3-dione (GS-371)

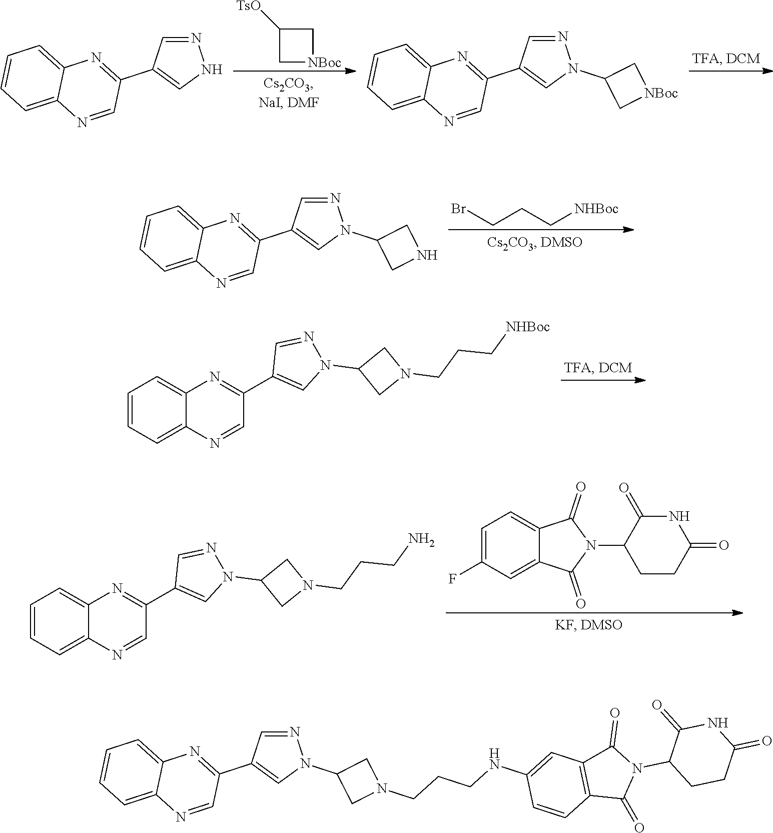

GS-371 was synthesized following the standard procedures for preparing GS-062 and GS-326 (6.6 mg, 15% yield) as a light-yellow solid. MS (ESI) m/z=565.4 [M+H]⁺.

### Example 330. 2-(2,6-Dioxopiperidin-3-yl)-5-((2-((3-(4-(pyridin-2-yl)piperazin-1-yl)phenyl)amino)ethyl)amino)isoindoline-1,3-dione (GS-372)

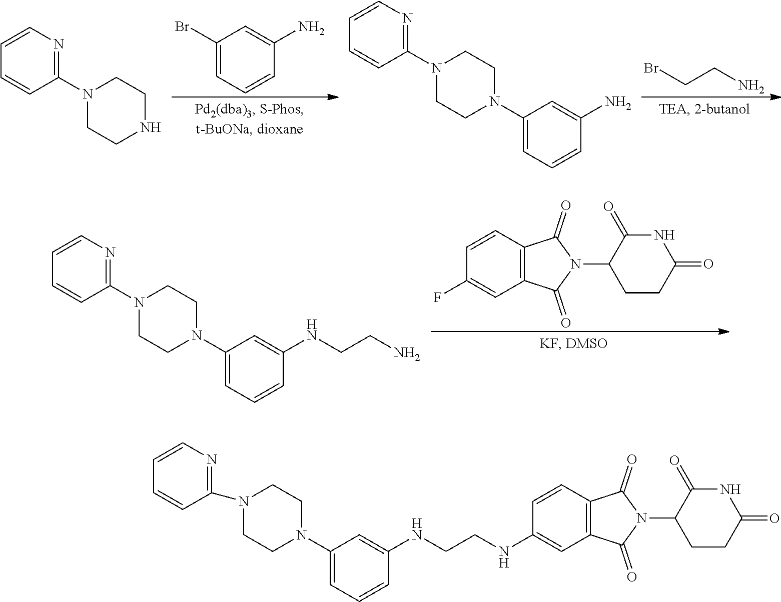

GS-372 was synthesized following the standard procedures for preparing GS-351 (2.7 mg, 18% yield) as a light-yellow solid. MS (ESI) m/z=554.3 [M+H]⁺.

### Example 331. 2-(2,6-Dioxopiperidin-3-yl)-5-((2-((3-(4-(quinoxalin-2-yl)-1H-pyrazol-1-yl)cyclobutyl)amino)ethyl)amino)isoindoline-1,3-dione (GS-373)

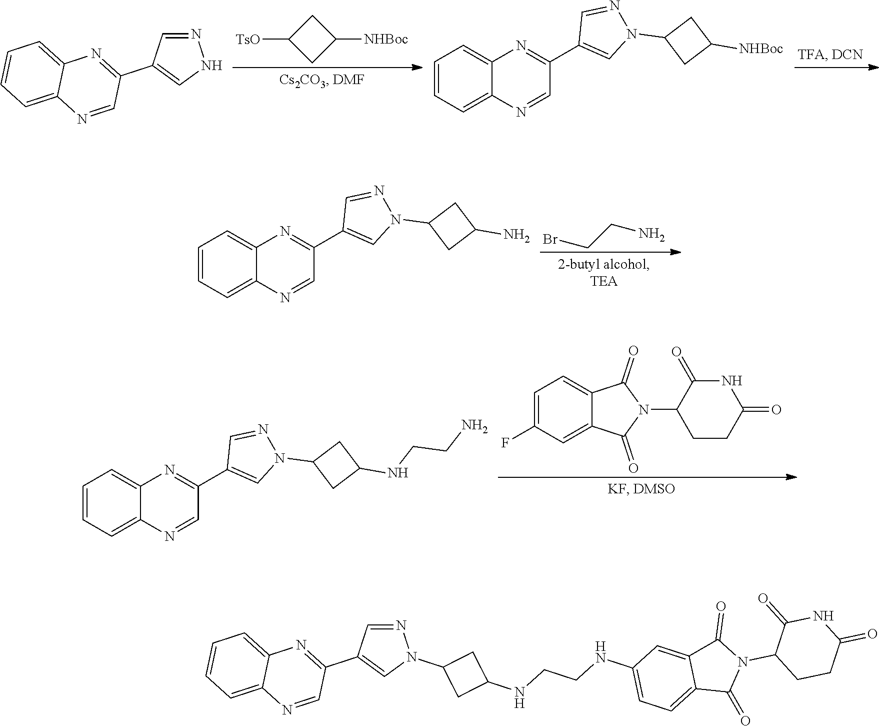

Step 1. Synthesis of tert-butyl (3-(4-(quinoxalin-2-yl)-1H-pyrazol-1-yl)cyclobutyl)carbamate

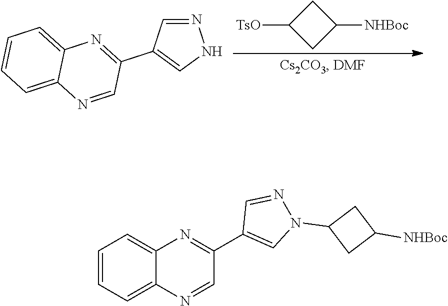 [SMILES: c1ccc2nc(-c3cn[nH]c3)cnc2c1 ; Cc1ccc(S(=O)(=O)OC2CC(NC(=O)OC(C)(C)C)C2)cc1 ; CC(C)(C)OC(=O)NC1CC(n2cc(-c3cnc4ccccc4n3)cn2)C1]

To a solution of 2-(1H-pyrazol-4-yl)quinoxaline (98 mg, 0.50 mmol) and 3-((tert-butoxycarbonyl)amino)cyclobutyl 4-methylbenzenesulfonate (170 mg, 0.50 mmol) in DMF (3 mL) were added Cs₂CO₃ (162 mg, 0.50 mmol) and NaI (30 mg, 0.20 mmol) at rt. Then the reaction mixture was stirred at 80° C. for 13 h. The mixture was purified by reverse-phase chromatography to provide the desired product (90 mg, 49% yield) as a light-yellow solid. MS (ESI) m/z=366.2 [M+H]⁺. The remaining steps were performed according to the standard procedures for preparing GS-351 to provide the desired product (9.5 mg, 55% yield) as a light-yellow solid. MS (ESI) m/z=565.3 [M+H]⁺.

### Example 332. 3-(1-Oxo-5-(6-(4-(quinoxalin-2-yl)-1H-pyrazol-1-yl)hexyl)isoindolin-2-yl)piperidine-2,6-dione (GS-374)

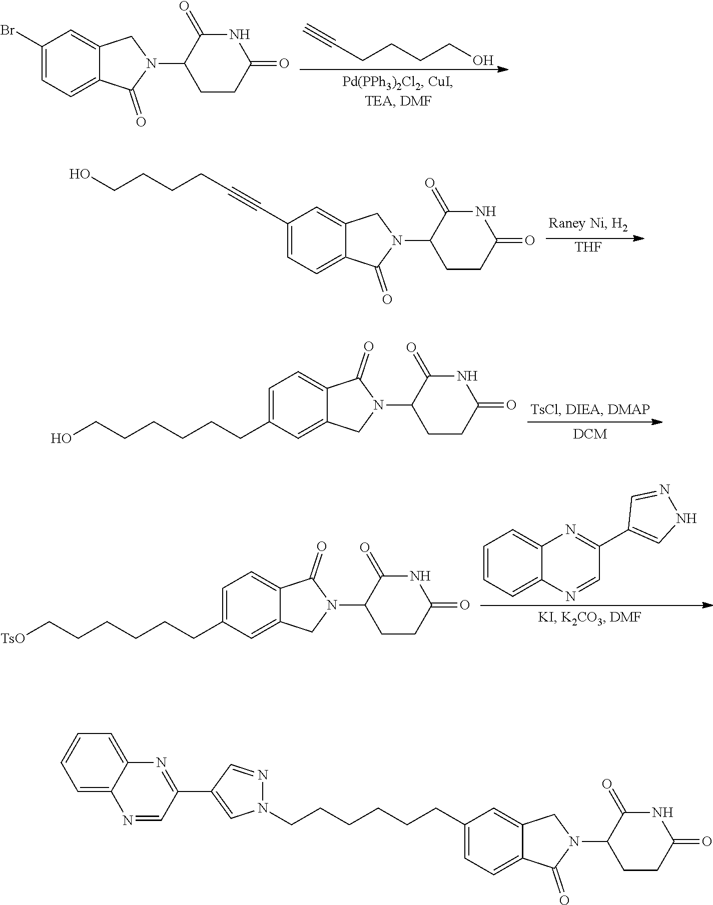

Step 1. Synthesis of 3-(5-(6-hydroxyhex-1-yn-1-yl)-1-oxoisoindolin-2-yl)piperidine-2,6 dione

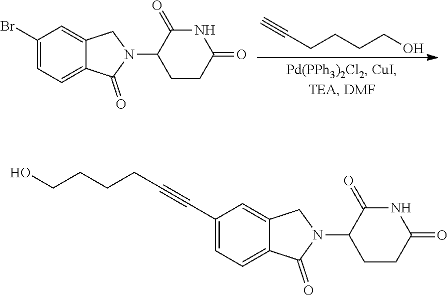

This step followed the procedure for preparing GS-369 to provide the desired product (45 mg, 86% yield) as a brown solid. MS (ESI) m/z=341.2 [M+H]⁺.

Step 2. Synthesis of 3-(5-(6-hydroxyhexyl)-1-oxoisoindolin-2-yl)piperidine-2,6-dione

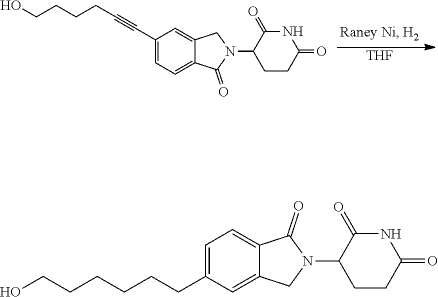 [SMILES: O=C1CCC(N2Cc3cc(C#CCCCCO)ccc3C2=O)C(=O)N1 ; O=C1CCC(N2Cc3cc(CCCCCCO)ccc3C2=O)C(=O)N1 ; "THF"]

A solution of 3-(5-(6-hydroxyhex-1-yn-1-yl)-1-oxoisoindolin-2-yl)piperidine-2,6-dione (45 mg, 0.044 mmol) and Raney Ni (10 mg) in THF (10 mL) was stirred at rt for 4 h under hydrogen balloon. Then the mixture was filtered through Celite and the filtrate was concentrated under reduced pressure. The resulting residue was purified by reverse-phase chromatography to provide the desired product (40 mg, 88% yield) as a light-yellow solid. MS (ESI) m/z=345.2 [M+H]⁺.

The remaining steps were performed according to the procedures for preparing GS-367 to provide the desired product (8 mg, 38% yield) as a white solid. MS (ESI) m/z=523.3 [M+H]⁺.

### Example 333. 3-(1-Oxo-5-(7-(4-(quinoxalin-2-yl)-1H-pyrazol-1-yl)heptyl)isoindolin-2-yl)piperidine-2,6-dione (GS-375)

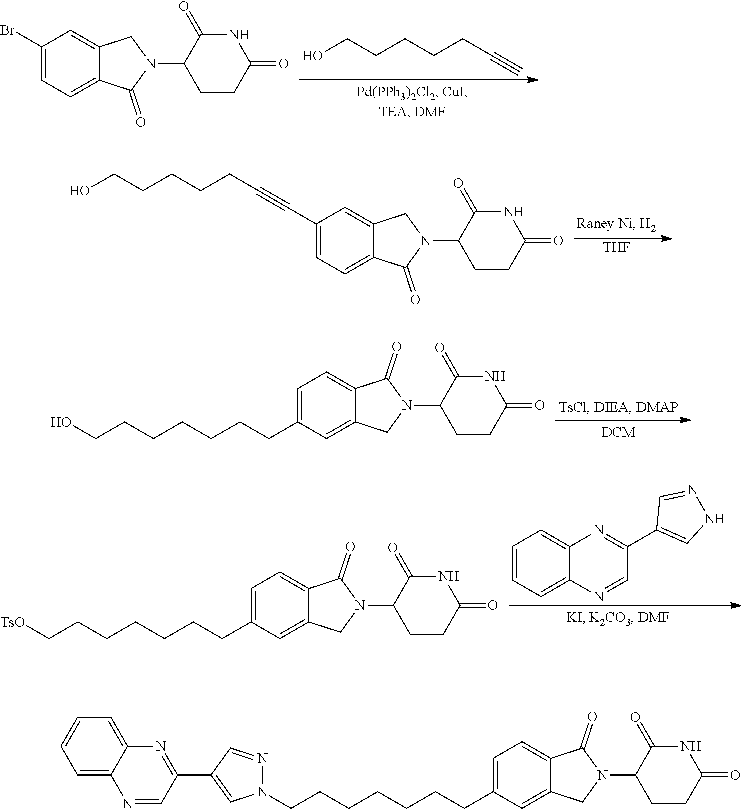

GS-375 was synthesized following the standard procedures for preparing GS-374 and GS-367 (10 mg, 37% yield) as a white solid. MS (ESI) m/z=537.4 [M+H]⁺.

### Example 334. 5-((6-(4-(8-Chloroquinoxalin-2-yl)-1H-pyrazol-1-yl)hexyl)amino)-2-(2,6-dioxopiperidin-3-yl)isoindoline-1,3-dione (GS-376)

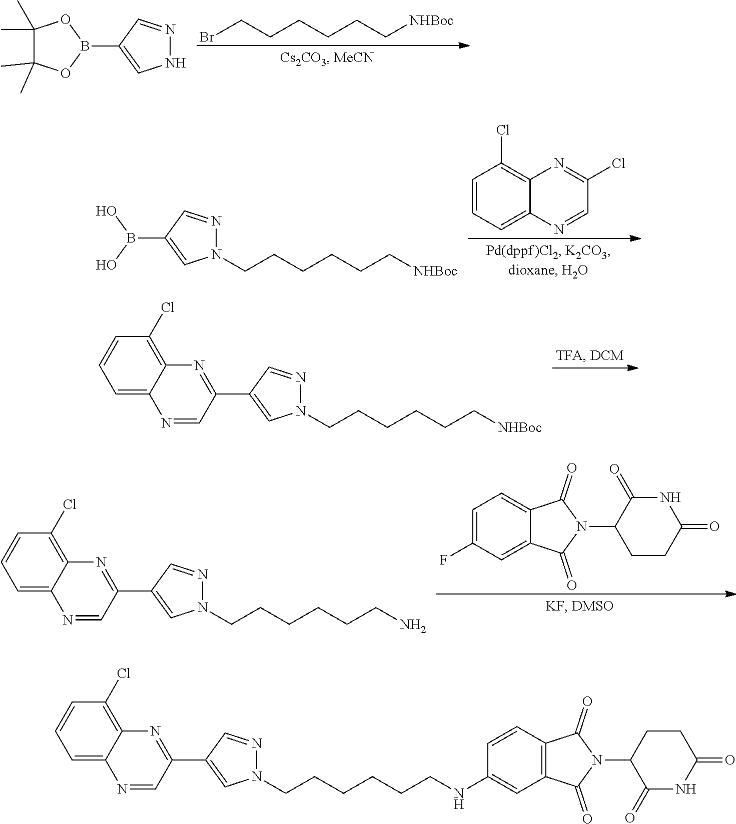

Step 1. Synthesis of (1-(6-((tert-butoxycarbonyl)amino)hexyl)-1H-pyrazol-4-yl)boronic acid

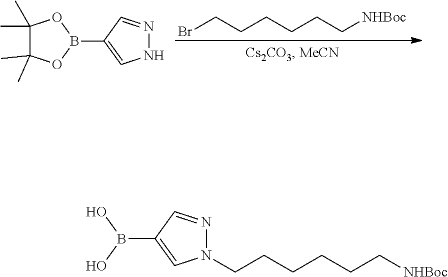 [SMILES: CC1(C)OB(c2cn[nH]c2)OC1(C)C ; CC(C)(C)OC(=O)NCCCCCCBr ; CC(C)(C)OC(=O)NCCCCCCn1cc(B(O)O)cn1]

To a solution of tert-butyl (6-bromohexyl)carbamate (100 mg, 0.51 mmol) in CH₃CN (10 mL) were added 4-(4,4,5,5-tetramethyl-1,3,2-dioxaborolan-2-yl)-1H-pyrazole (40 mg, 0.46 mmol) and Cs₂CO₃ (196 mg, 0.6 mmol) at rt. The reaction mixture was stirred at 80° C. under inert atmosphere for 1 h. After cooling down rt, the mixture was filtered. The filtrate was concentrated and purified by reverse-phase chromatography to provide the desired product (58 mg, 36% yield) as a light-yellow solid. MS (ESI) m/z=312.0 [M+H]⁺.

Step 2. Synthesis of tert-butyl (6-(4-(8-chloroquinoxalin-2-yl)-1H-pyrazol-1-yl)hexyl)carbamate

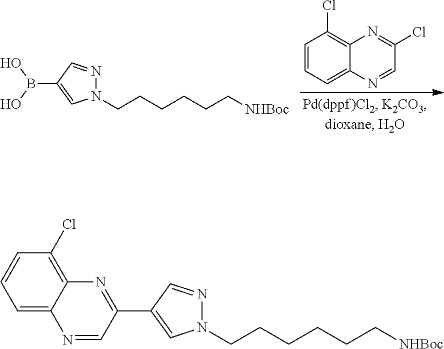 [SMILES: CC(C)(C)OC(=O)NCCCCCCn1cc(B(O)O)cn1 ; Clc1cnc2cccc(Cl)c2n1 ; CC(C)(C)OC(=O)NCCCCCCn1cc(-c2cnc3cccc(Cl)c3n2)cn1]

To a solution of 2,8-dichloroquinoxaline (30.0 mg, 0.15 mmol) in dioxane (5 mL) and H₂O (1 mL) were added (1-(6-((tert-butoxycarbonyl)amino)hexyl)-1H-pyrazol-4-yl)boronic acid (47.4 mg, 0.15 mmol), Pd(dppf)Cl₂ (5.5 mg, 0.008 mmol) and K₂CO₃ (62.4 mg, 0.45 mmol) at rt. The reaction mixture was stirred at 100° C. under inert atmosphere for 2 h. After cooling down to rt, the mixture was filtered. The filtrate was concentrated and purified by reverse-phase chromatography to provide the desired product (30.7 mg, 74% yield) as a light-yellow solid. MS (ESI) m/z=430.2 [M+H]⁺. The remaining steps were performed according to the standard procedures for preparing GS-326 to provide the desired product (6.8 mg, 17% yield) as a light-yellow solid. MS (ESI) m/z=586.2 [M+H]⁺.

### Example 335. 2-(2,6-Dioxopiperidin-3-yl)-5-((6-(4-(8-fluoroquinoxalin-2-yl)-1H-pyrazol-1-yl)hexyl)amino)isoindoline-1,3-dione (GS-377)

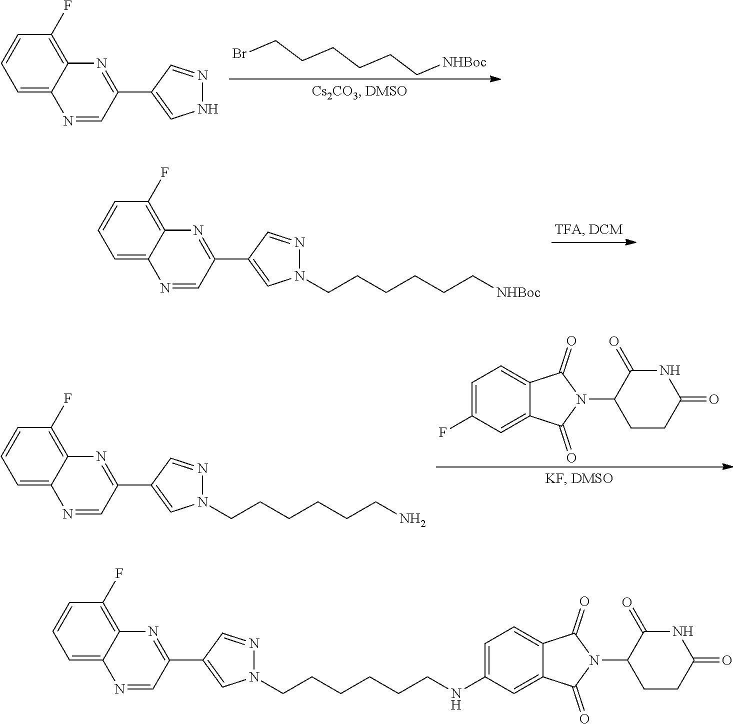 [SMILES: Fc1cccc2ncc(-c3cn[nH]c3)nc12 ; CC(C)(C)OC(=O)NCCCCCCBr ; CC(C)(C)OC(=O)NCCCCCCn1cc(-c2cnc3cccc(F)c3n2)cn1 ; NCCCCCCn1cc(-c2cnc3cccc(F)c3n2)cn1 ; O=C1CCC(N2C(=O)c3ccc(F)cc3C2=O)C(=O)N1 ; O=C1CCC(N2C(=O)c3ccc(NCCCCCCn4cc(-c5cnc6cccc(F)c6n5)cn4)cc3C2=O)C(=O)N1]

GS-377 was synthesized following the standard procedures for preparing GS-326 (8.0 mg, 43% yield) as a light-yellow solid. MS (ESI) m/z=570.3 [M+H]⁺.

### Example 336. 3-(1-Oxo-4-((4-((3-(4-(4-(quinoxalin-2-yl)-1H-pyrazol-1-yl)piperidin-1-yl)phenyl)amino)cyclohexyl)amino)isoindolin-2-yl)piperidine-2,6-dione (GS-378)

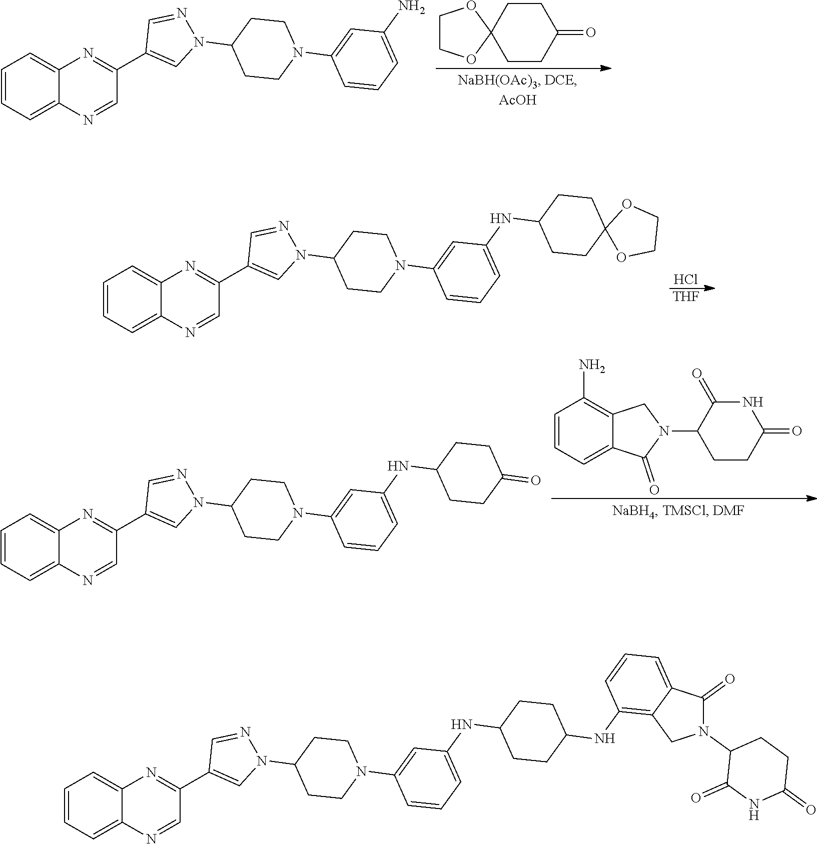

Step 1. Synthesis of N-(3-(4-(4-(quinoxalin-2-yl)-1H-pyrazol-1-yl)piperidin-1-yl)phenyl)-1,4-dioxaspiro[4.5]decan-8-amine

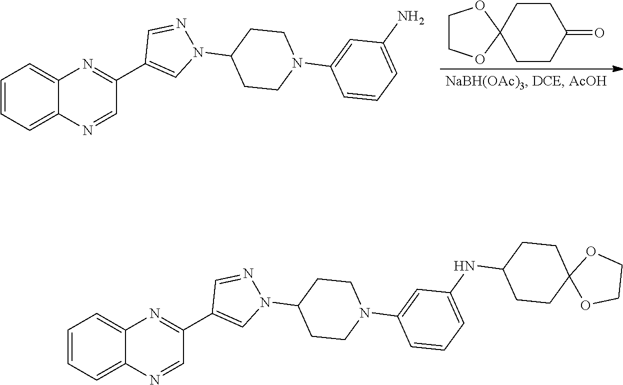

To a solution of 3-(4-(4-(quinoxalin-2-yl)-1H-pyrazol-1-yl)piperidin-1-yl)aniline (40 mg, 0.11 mmol) in DCE (5 mL) and AcOH (1 mL) were added 1,4-dioxaspiro[4.5]decan-8-one (33 mg, 0.22 mmol) and NaBH(OAc)₃ (46 mg, 0.22 mmol) at 0° C. The reaction mixture was stirred at rt for 3 h. Then the reaction was quenched with water and extracted with DCM (3×10 mL). The combined organic layers were washed with brine, dried over anhydrous sodium sulfate, filtered, and concentrated under reduced pressure. The resulting residue was purified by prep-TLC to provide the desired product (48 mg, 86% yield) as a white solid. MS (ESI) m/z=511.4 [M+H]⁺.

Step 2. Synthesis of 4-((3-(4-(4-(quinoxalin-2-yl)-1H-pyrazol-1-yl)piperidin-1-yl)phen 1 amino)cyclohexan-1-one

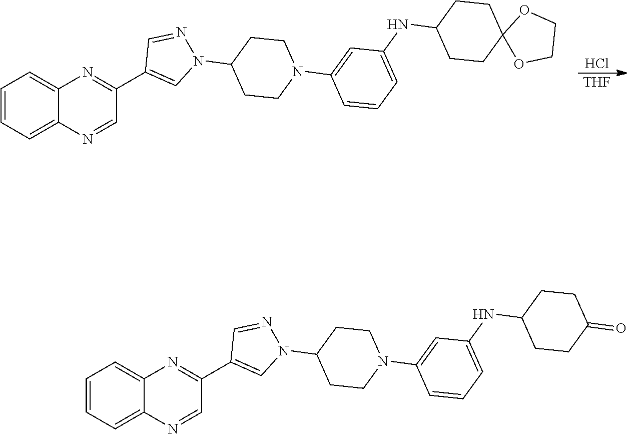 [SMILES: c1cc(NC2CCC3(CC2)OCCO3)cc(N2CCC(n3cc(-c4cnc5ccccc5n4)cn3)CC2)c1 ; Cl ; O=C1CCC(Nc2cccc(N3CCC(n4cc(-c5cnc6ccccc6n5)cn4)CC3)c2)CC1 ; "THF"]

To a solution of N-(3-(4-(4-(quinoxalin-2-yl)-1H-pyrazol-1-yl)piperidin-1-yl)phenyl)-1,4-dioxaspiro[4.5]decan-8-amine (48 mg, 0.09 mmol) in THF (5 mL) was added 1M HCl (2 mL) at 0° C. The reaction mixture was stirred at rt for 16 h, before it was quenched with water and extracted with DCM (3×10 mL). The combined organic layers were dried over anhydrous sodium sulfate, filtered, and concentrated to provide the desired product (30 mg, 76% yield) as a white solid. MS (ESI) m/z=467.3 [M+H]⁺.

Step 3. Synthesis of 3-(1-oxo-4-((4-((3-(4-(4-(quinoxalin-2-yl)-1H-pyrazol-1-yl)piperidin-1-yl)phenyl)amino)cyclohexyl)amino)isoindolin-2-yl)piperidine-2,6-dione

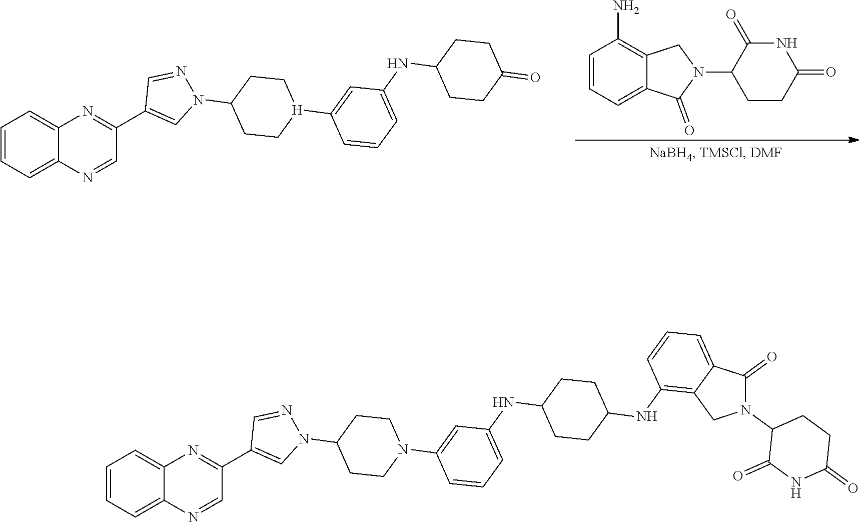

To a solution of 4-((3-(4-(4-(quinoxalin-2-yl)-1H-pyrazol-1-yl)piperidin-1-yl)phenyl)amino)cyclohexan-1-one (30 mg, 0.06 mmol) in DMF (3 mL) were added 3-(4-amino-1-oxoisoindolin-2-yl)piperidine-2,6-dione (24 mg, 0.09 mmol) and TMSCl (20 mg, 0.18 mmol) at 0° C. After stirring at rt for 30 min, NaBH₄ (5 mg, 0.12 mmol) was added. The reaction mixture was stirred at rt for another 3 h, before it was purified by reverse-phase chromatography, followed by prep-TLC to provide the desired product (18.3 mg, 44% yield) as a white solid. MS (ESI) m/z=710.4 [M+H]⁺.

### Example 337. N-(4-((2-(2,6-Dioxopiperidin-3-yl)-1,3-dioxoisoindolin-5-yl)amino)butyl)-4-(4-(quinoxalin-2-yl)-1H-pyrazol-1-yl)piperidine-1-carboxamide (GS-379)

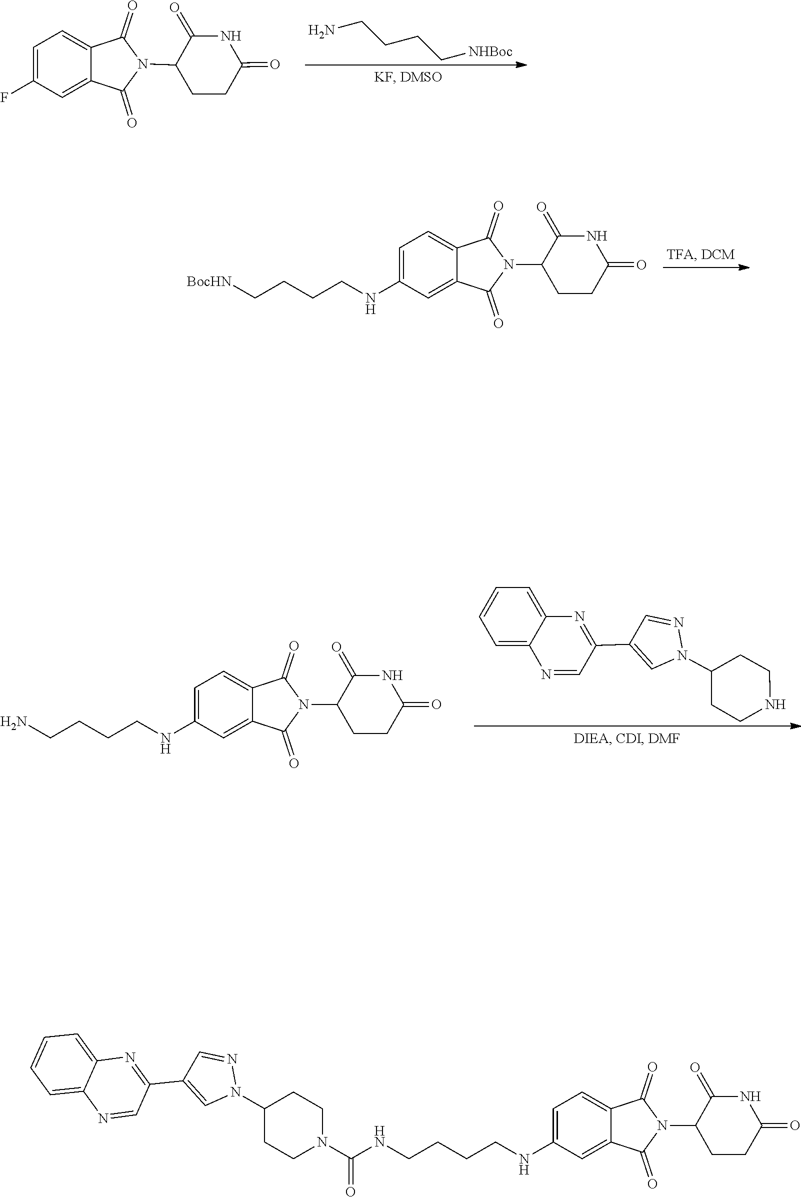 [SMILES: O=C1CCC(N2C(=O)c3ccc(F)cc3C2=O)C(=O)N1 ; CC(C)(C)OC(=O)NCCCCN ; CC(C)(C)OC(=O)NCCCCNc1ccc2c(c1)C(=O)N(C1CCC(=O)NC1=O)C2=O ; NCCCCNc1ccc2c(c1)C(=O)N(C1CCC(=O)NC1=O)C2=O ; c1ccc2nc(-c3cnn(C4CCNCC4)c3)cnc2c1 ; O=C1CCC(N2C(=O)c3ccc(NCCCCNC(=O)N4CCC(n5cc(-c6cnc7ccccc7n6)cn5)CC4)cc3C2=O)C(=O)N1]

Step 1. Synthesis of tert-butyl (4-((2-(2,6-dioxopiperidin-3-yl)-1,3-dioxoisoindolin-5-yl)amino)butyl)carbamate

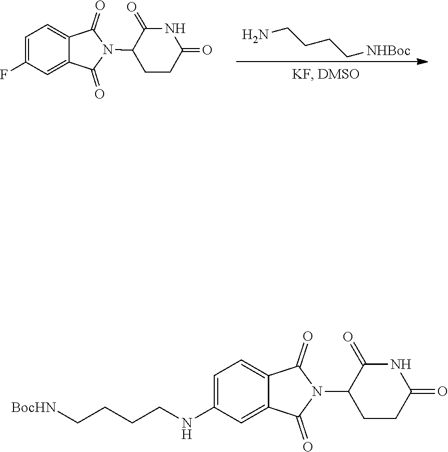 [SMILES: O=C1CCC(N2C(=O)c3ccc(F)cc3C2=O)C(=O)N1 ; CC(C)(C)OC(=O)NCCCCN ; CC(C)(C)OC(=O)NCCCCNc1ccc2c(c1)C(=O)N(C1CCC(=O)NC1=O)C2=O]

To a solution of 2-(2,6-dioxopiperidin-3-yl)-5-fluoroisoindoline-1,3-dione (55 mg, 0.20 mmol) and tert-butyl (4-aminobutyl)carbamate (36 mg, 0.20 mmol) in DMSO (2 mL) was added KF (35 mg, 0.60 mmol) at rt. Then the reaction mixture was stirred at 100° C. for 3 h. The mixture was purified by reverse-phase chromatography to provide the desired product (50 mg, 56% yield) as a light-yellow solid. MS (ESI) m/z=445.2 [M+H]⁺.

Step 2. Synthesis of 5-((4-aminobutyl)amino)-2-(2,6-dioxopiperidin-3-yl)isoindoline-1,3-dione

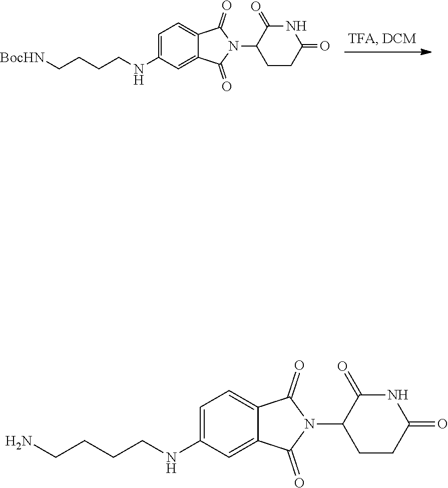 [SMILES: CC(C)(C)OC(=O)NCCCCNc1ccc2c(c1)C(=O)N(C1CCC(=O)NC1=O)C2=O ; NCCCCNc1ccc2c(c1)C(=O)N(C1CCC(=O)NC1=O)C2=O]

A solution of tert-butyl (4-((2-(2,6-dioxopiperidin-3-yl)-1,3-dioxoisoindolin-5-yl)amino)butyl)carbamate (50 mg, 0.11 mmol) in TFA/DCM (1:1, 2 mL) was stirred at rt for 1 h. The resulting mixture was concentrated to provide the crude product (32 mg, 84% yield) as a light-yellow solid. This compound was used directly in the next step without further purification. MS (ESI) m/z=345.2 [M+H]⁺.

Step 3. Synthesis of N-(4-((2-(2,6-dioxopiperidin-3-yl)-1,3-dioxoisoindolin-5-yl)amino)butyl)-4-(4-(quinoxalin-2-yl)-1H-pyrazol-1-yl)piperidine-1-carboxamide

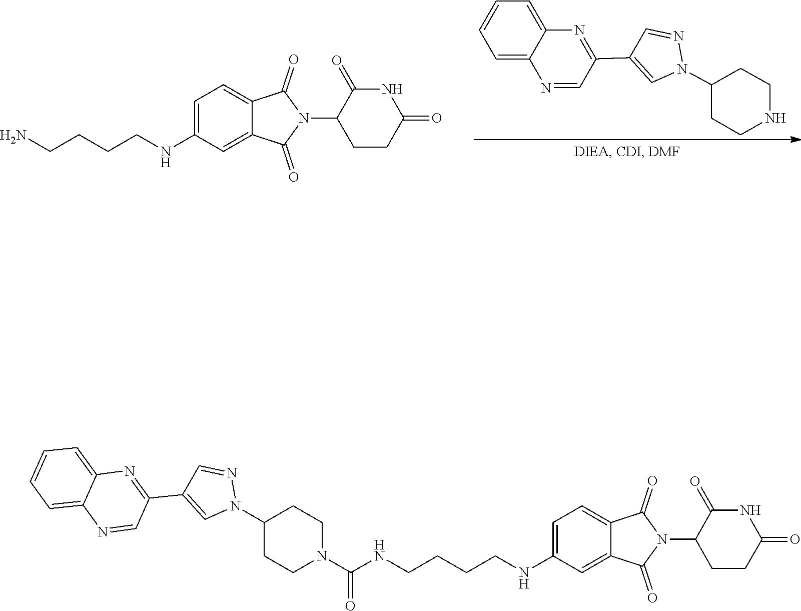 [SMILES: NCCCCNc1ccc2c(c1)C(=O)N(C1CCC(=O)NC1=O)C2=O ; c1ccc2nc(-c3cnn(C4CCNCC4)c3)cnc2c1 ; O=C1CCC(N2C(=O)c3ccc(NCCCCNC(=O)N4CCC(n5cc(-c6cnc7ccccc7n6)cn5)CC4)cc3C2=O)C(=O)N1]

To a solution of 5-((4-aminobutyl)amino)-2-(2,6-dioxopiperidin-3-yl)isoindoline-1,3-dione (32 mg, 0.093 mmol) and 2-(1-(piperidin-4-yl)-1H-pyrazol-4-yl)quinoxaline (26 mg, 0.093 mmol) in DMF (2 mL) were added CDI (16 mg, 0.10 mmol) and DIEA (13 mg, 0.10 mmol) at rt. The reaction mixture was stirred at 80° C. for 1 h, before it was purified by reverse-phase chromatography to provide the desired product (15 mg, 25% yield) as a light-yellow solid. MS (ESI) m/z=650.3 [M+H]⁺.

### Example 338. 2-(2,6-Dioxopiperidin-3-yl)-5-((6-(2-methyl-4-(quinoxalin-2-yl)-1H-imidazol-1-yl)hexyl)amino)isoindoline-1,3-dione (GS-380)

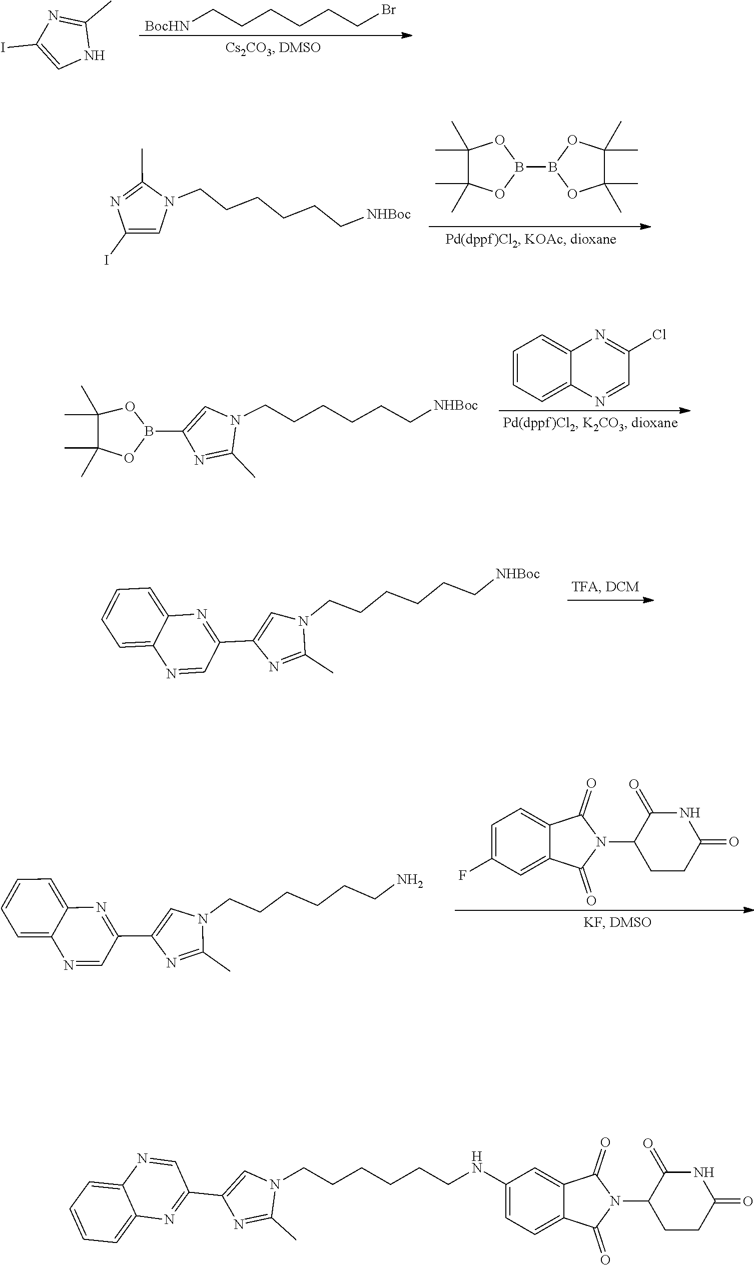

Step 1. Synthesis of tert-butyl (6-(4-iodo-2-methyl-1H-imidazol-1-yl)hexyl)carbamate

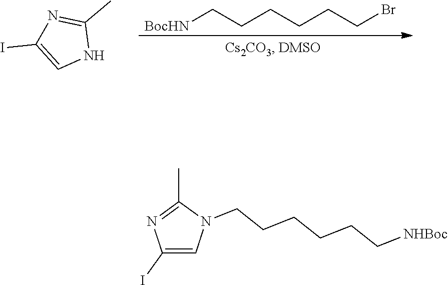

To a solution of 4-iodo-2-methyl-1H-imidazole (40 mg, 0.20 mmol) and tert-butyl (6-bromohexyl)carbamate (56 mg, 0.20 mmol) in DMSO (3 mL) was added Cs₂CO₃ (130 mg, 0.40 mmol) at rt. Then the reaction mixture was stirred at 100° C. for 4 h. The mixture was purified by reverse-phase chromatography to provide the desired product (50 mg, 61% yield) as a light-yellow solid. MS (ESI) m/z=408.1 [M+H]⁺.

Step 2. Synthesis of tert-butyl (6-(2-methyl-4-(4,4,5,5-tetramethyl-1,3,2-dioxaborolan-2-yl)-1H-imidazol-1-yl)hexyl)carbamate

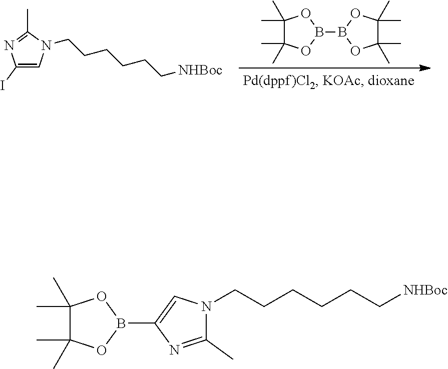

A mixture of tert-butyl (6-(4-iodo-2-methyl-1H-imidazol-1-yl)hexyl)carbamate (40 mg, 0.10 mmol), 4,4,4′,4′,5,5,5′,5′-octamethyl-2,2′-bi(1,3,2-dioxaborolane) (25 mg, 0.03 mmol), Pd(dppf)Cl₂ (14 mg, 0.02 mmol) and KOAc (25 mg, 0.30 mmol) in dioxane (5 mL) was stirred at 100° C. for 3 h. Then the mixture was concentrated and purified by reverse-phase chromatography to provide the desired product (18 mg, 44% yield) as a light-yellow solid. MS (ESI) m/z=408.3 [M+H]⁺.

Step 3. Synthesis of tert-butyl (6-(2-methyl-4-(quinoxalin-2-yl)-1H-imidazol-1-yl)hexyl)carbamate

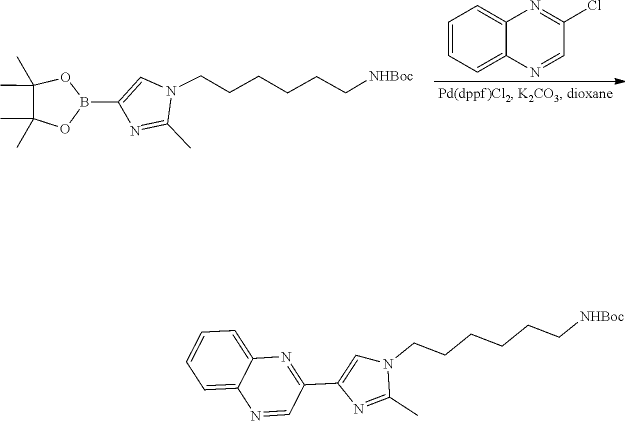

To a solution of tert-butyl (6-(2-methyl-4-(4,4,5,5-tetramethyl-1,3,2-dioxaborolan-2-yl)-1H-imidazol-1-yl)hexyl)carbamate (18 mg, 0.04 mmol) in dioxane (10 mL) and H₂O (1 mL) were added 2-chloroquinoxaline (8 mg, 0.05 mmol), Pd(dppf)Cl₂ (7 mg, 0.01 mmol) and K₂CO₃ (32 mg, 0.10 mmol) at rit. Then the mixture was stirred at 100° C. under N₂ atmosphere for 2 h. The mixture was filtered, concentrated and purified by reverse-phase chromatography to provide the desired product (15 mg, 82% yield) as a light-yellow solid. MS (ESI) m/z=410.3 [M+H]⁺.

Step 4. Synthesis of 6-(2-methyl-4-(quinoxalin-2-yl)-1H-imidazol-1-yl)hexan-1-amine

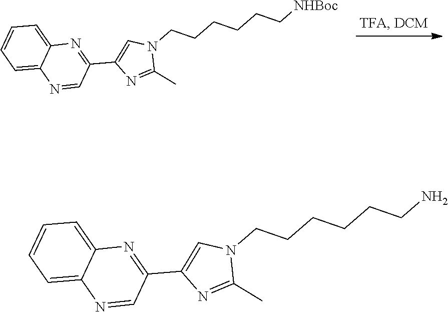 [SMILES: Cc1nc(-c2cnc3ccccc3n2)cn1CCCCCCNC(=O)OC(C)(C)C ; Cc1nc(-c2cnc3ccccc3n2)cn1CCCCCCN]

A mixture of tert-butyl (6-(2-methyl-4-(quinoxalin-2-yl)-1H-imidazol-1-yl)hexyl)carbamate (15 mg, 0.18 mmol) in TFA/DCM (1:1, 2 mL) was stirred at rt for 1 h. The resulting mixture was concentrated to provide the crude product (10 mg, 83% yield) as a light-yellow solid. This compound was used directly in the next step without further purification. MS (ESI) m/z=310.2 [M+H]⁺.

Step 5. Synthesis of 2-(2,6-dioxopiperidin-3-yl)-5-((6-(2-methyl-4-(quinoxalin-2-yl)-1H-imidazol-1-yl)hexyl)amino)isoindoline-1,3-dione

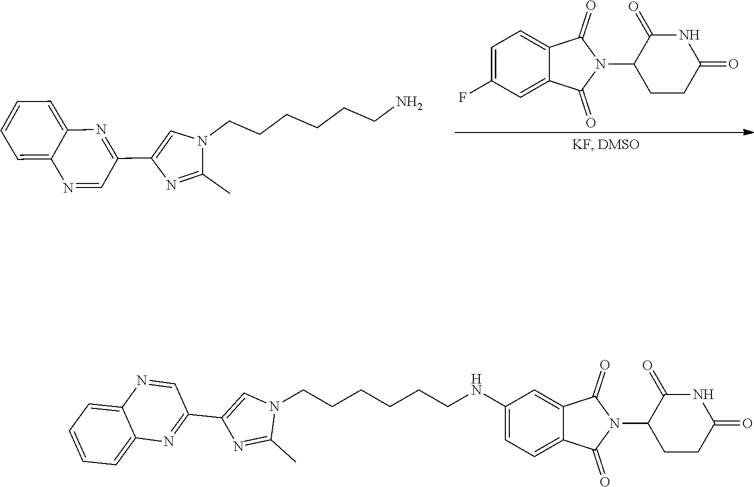

To a solution of 6-(2-methyl-4-(quinoxalin-2-yl)-1H-imidazol-1-yl)hexan-1-amine (10 mg, 0.03 mmol) and 2-(2,6-dioxopiperidin-3-yl)-5-fluoroisoindoline-1,3-dione (8 mg, 0.03 mmol) in DMSO (2 mL) was added KF (6 mg, 0.10 mmol) at rt. Then the reaction mixture was stirred at 100° C. for 1 h. The mixture was purified by reverse-phase chromatography to provide the desired product (5 mg, 30% yield) as a light-yellow solid. MS (ESI) m/z=566.3 [M+H]⁺.

### Example 339. 2-(2,6-Dioxopiperidin-3-yl)-5-((6-(4-(8-morpholinoquinoxalin-2-yl)-1H-pyrazol-1-yl)hexyl)amino)isoindoline-1,3-dione (GS-381)

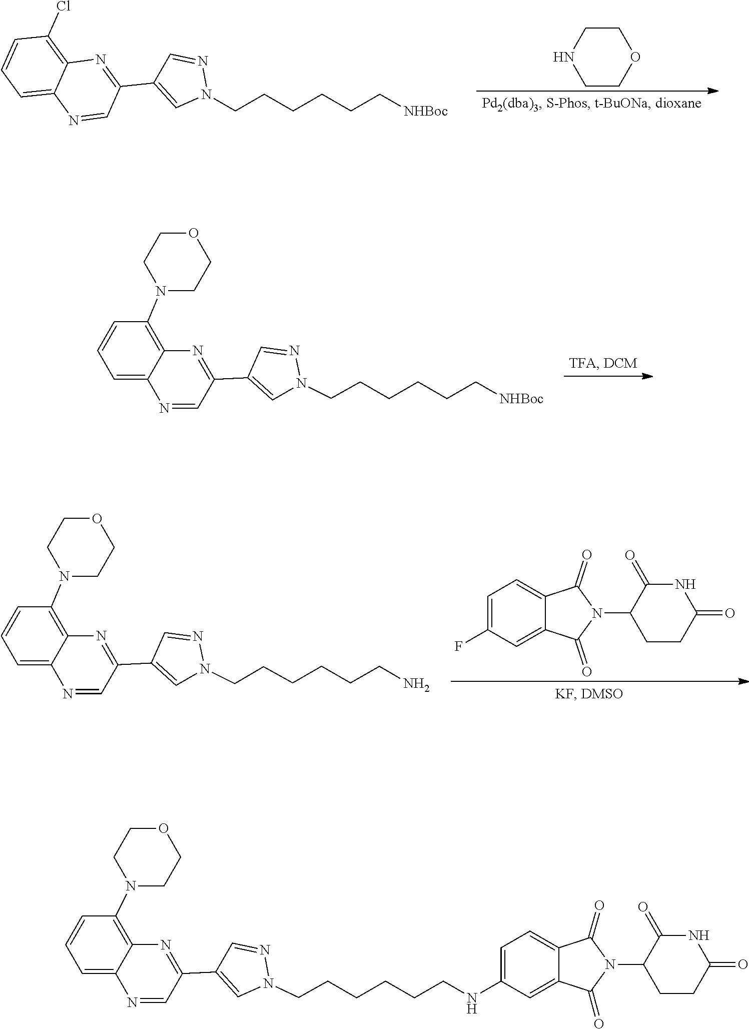 [SMILES: CC(C)(C)OC(=O)NCCCCCCn1cc(-c2cnc3cccc(Cl)c3n2)cn1 ; C1COCCN1 ; CC(C)(C)OC(=O)NCCCCCCn1cc(-c2cnc3cccc(N4CCOCC4)c3n2)cn1 ; NCCCCCCn1cc(-c2cnc3cccc(N4CCOCC4)c3n2)cn1 ; O=C1CCC(N2C(=O)c3ccc(F)cc3C2=O)C(=O)N1 ; O=C1CCC(N2C(=O)c3ccc(NCCCCCCn4cc(-c5cnc6cccc(N7CCOCC7)c6n5)cn4)cc3C2=O)C(=O)N1]

Step 1. Synthesis of tert-butyl (6-(4-(8-morpholinoquinoxalin-2-yl)-1H-pyrazol-1-yl)hexyl)carbamate

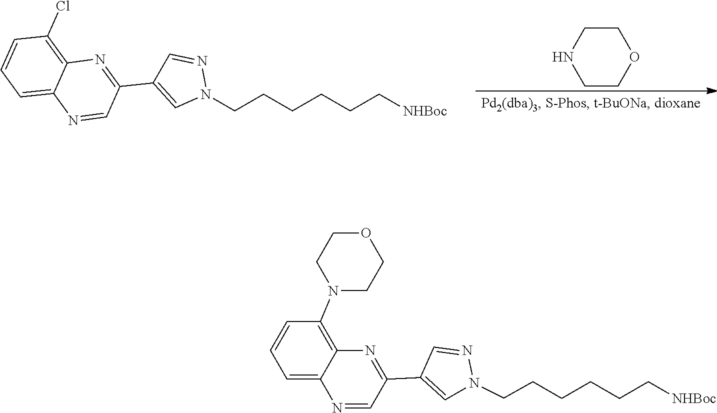 [SMILES: CC(C)(C)OC(=O)NCCCCCCn1cc(-c2cnc3cccc(Cl)c3n2)cn1 ; C1COCCN1 ; CC(C)(C)OC(=O)NCCCCCCn1cc(-c2cnc3cccc(N4CCOCC4)c3n2)cn1]

To a solution of tert-butyl (6-(4-(8-chloroquinoxalin-2-yl)-1H-pyrazol-1-yl)hexyl)carbamate (100 mg, 0.23 mmol) in dioxane (5 mL) were added morpholine (40 mg, 0.46 mmol), Pd₂(dba)₃ (7 mg, 0.01 mmol), S-Phos (80 mg, 0.20 mmol) and t-BuONa (40 mg, 0.40 mmol) at rt. Then the mixture was stirred at 100° C. under inert atmosphere for 1 h. After cooling down to rt, the mixture was filtered. The filtrate was concentrated and purified by reverse-phase chromatography to provide the desired product (22 mg, 25% yield) as a light-yellow solid. MS (ESI) m/z=481.3 [M+H]⁺.

Step 2. Synthesis of 6-(4-(8-morpholinoquinoxalin-2-yl)-1H-pyrazol-1-yl)hexan-1-amine

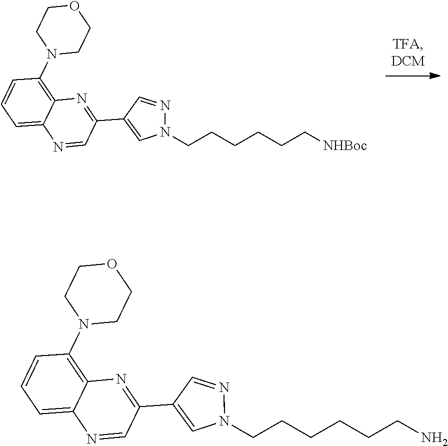 [SMILES: CC(C)(C)OC(=O)NCCCCCCn1cc(-c2cnc3cccc(N4CCOCC4)c3n2)cn1 ; NCCCCCCn1cc(-c2cnc3cccc(N4CCOCC4)c3n2)cn1]

A solution of tert-butyl (6-(4-(8-morpholinoquinoxalin-2-yl)-1H-pyrazol-1-yl)hexyl)carbamate (20 mg, 0.04 mmol) in DCM (4 mL) and TFA (2 mL) was stirred at rt for 4 h. The solution was concentrated under reduced pressure. The resulting residue was purified by reverse-phase chromatography to provide the desired product (14 mg, 92% yield) as a light-yellow solid. MS (ESI) m/z=381.3 [M+H]⁺.

Step 3. Synthesis of 2-(2,6-dioxopiperidin-3-yl)-5-((6-(4-(8-morpholinoquinoxalin-2-yl)-1H-pyrazol-1-yl)hexyl)amino)isoindoline-1,3-dione

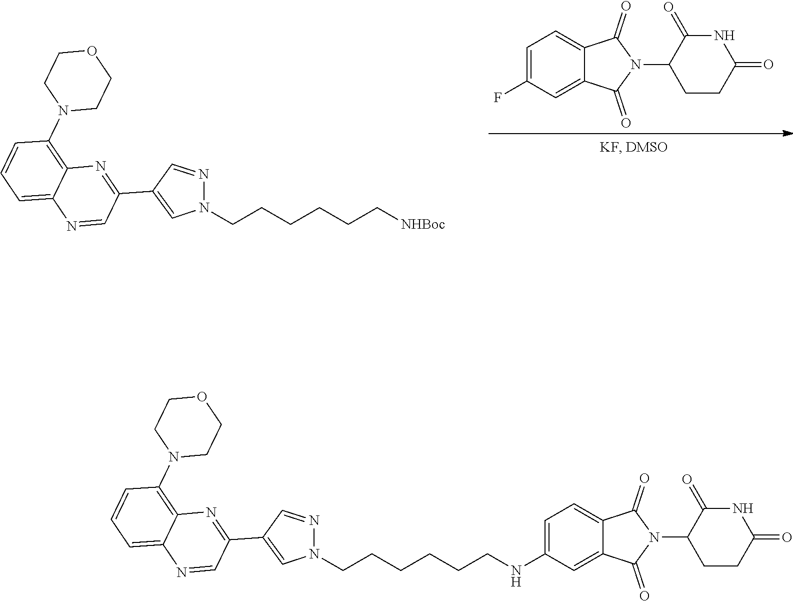 [SMILES: CC(C)(C)OC(=O)NCCCCCCn1cc(-c2cnc3cccc(N4CCOCC4)c3n2)cn1 ; O=C1CCC(N2C(=O)c3ccc(F)cc3C2=O)C(=O)N1 ; O=C1CCC(N2C(=O)c3ccc(NCCCCCCn4cc(-c5cnc6cccc(N7CCOCC7)c6n5)cn4)cc3C2=O)C(=O)N1]

To a solution of 6-(4-(8-morpholinoquinoxalin-2-yl)-1H-pyrazol-1-yl)hexan-1-amine (14 mg, 0.03 mmol) in DMSO (2 mL) were added 2-(2,6-dioxopiperidin-3-yl)-5-fluoroisoindoline-1,3-dione (10 mg, 0.03 mmol) and KF (5 mg, 0.09 mmol) at rt. Then the mixture was stirred at 130° C. under inert atmosphere and microwave irradiation for 30 min. After cooling down to rt, the mixture was purified by reverse-phase chromatography to provide the desired product (5 mg, 25% yield) as a light-yellow solid. MS (ESI) m/z=637.3 [M+H]⁺.

### Example 340. 2-(2,6-Dioxopiperidin-3-yl)-5-((2-((3-(4-(quinoxalin-2-yl)-1H-pyrazol-1-yl)phenyl)amino)ethyl)amino)isoindoline-1,3-dione (GS-382)

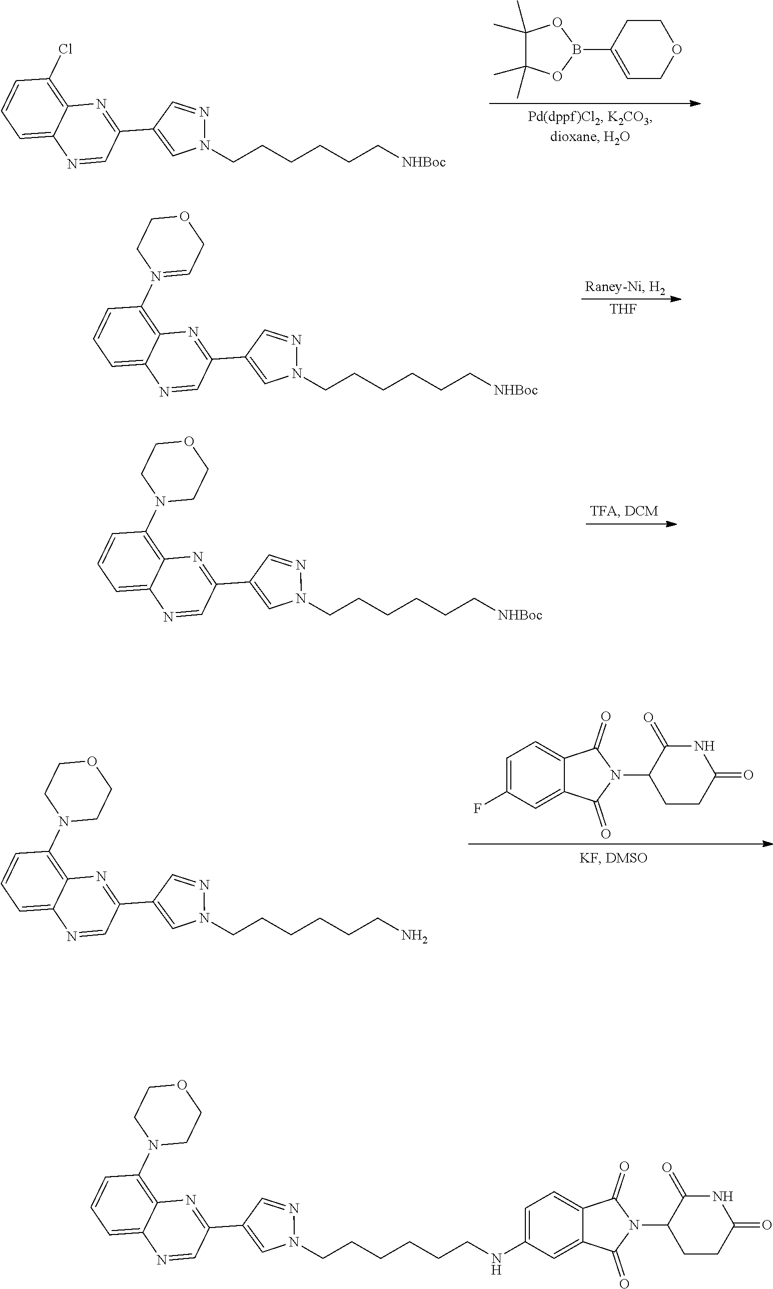

Step 1. Synthesis of tert-butyl (6-(4-(8-(3,6-dihydro-2H-pyran-4-yl)quinoxalin-2-yl)-1H-pyrazol-1-yl)hexyl)carbamate

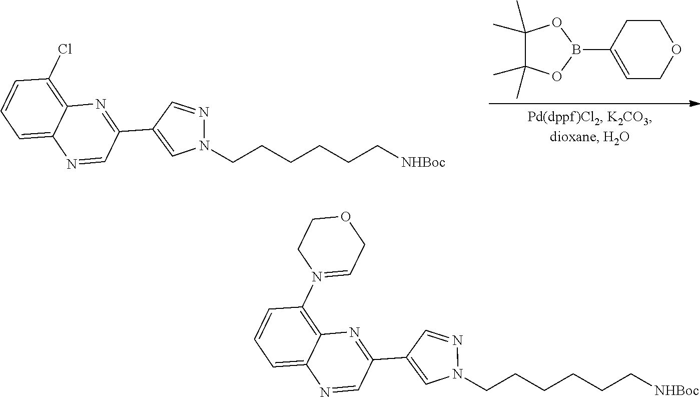

To a solution of 2-(3,6-dihydro-2H-pyran-4-yl)-4,4,5,5-tetramethyl-1,3,2-dioxaborolane (105 mg, 0.50 mmol) in dioxane (10 mL) and H₂O (1 mL) were added tert-butyl (6-(4-(8-chloroquinoxalin-2-yl)-1H-pyrazol-1-yl)hexyl)carbamate (220 mg, 0.50 mmol), Pd(dppf)Cl₂ (33 mg, 0.05 mmol) and K₂CO₃ (69 mg, 0.5 mmol) at rt. Then the mixture was stirred at 100° C. under inert atmosphere for 2 h. After cooling down to rt, the mixture was filtered. The filtrate was concentrated and purified by reverse-phase chromatography to provide the desired product (120 mg, 50% yield) as a light-yellow solid. MS (ESI) m/z=478.3 [M+H]⁺.

Step 2. Synthesis of tert-butyl (6-(4-(8-(tetrahydro-2H-pyran-4-yl)quinoxalin-2-yl)-1H-pyrazol-1-yl)hexyl)carbamate

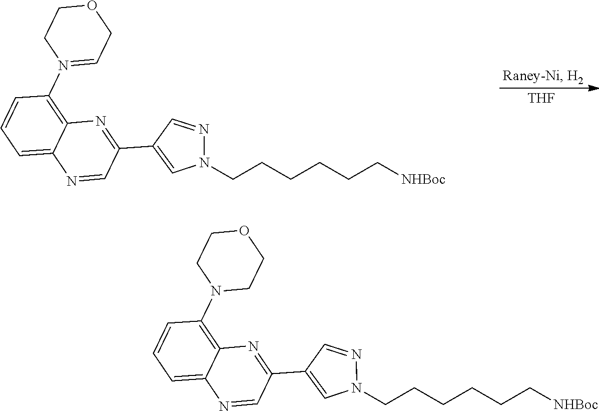

To a solution of tert-butyl (6-(4-(8-(3,6-dihydro-2H-pyran-4-yl)quinoxalin-2-yl)-1H-pyrazol-1-yl)hexyl)carbamate (120 mg, 0.25 mmol) in THF (10 mL) was added Raney-Nickel (50 mg). The mixture was purged with hydrogen and stirred at rt under hydrogen balloon for 4 h. Then MnO₂ (87 mg, 1.00 mmol) was added and the solution was stirred at rt under air for 1 h. The reaction mixture was filtered, concentrated and purified by reverse-phase chromatography to provide the desired product (60 mg, 51% yield) as a white solid. MS (ESI) m/z=480.3 [M+H]⁺.

The remaining steps were performed according to the procedures for preparing GS-381 to provide the desired product (15 mg, 56% yield) as a light-yellow solid. MS (ESI) m/z=636.3 [M+H]⁺

### Example 341. 2-(2,6-Dioxopiperidin-3-yl)-5-((6-(4-(8-(4-methylpiperazin-1-yl)quinoxalin-2-yl)-1H-pyrazol-1-yl)hexyl)amino)isoindoline-1,3-dione (GS-383)

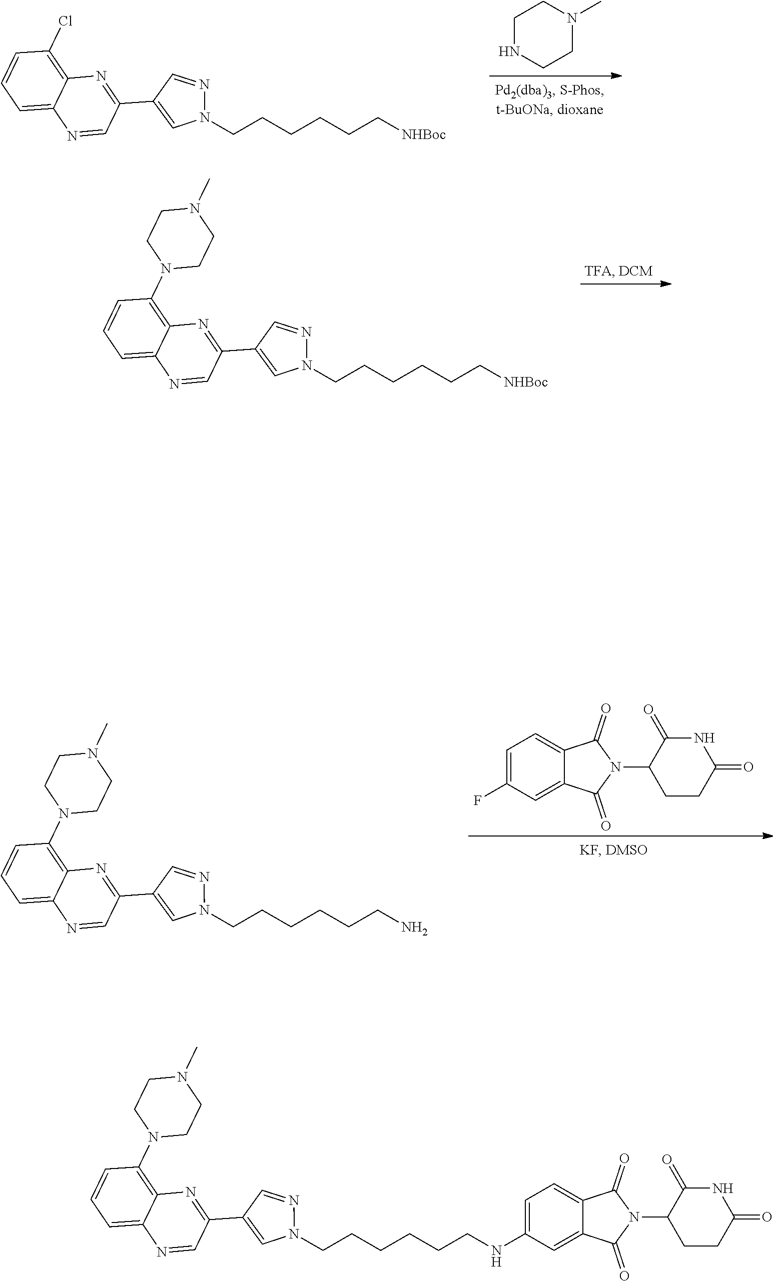

GS-383 was synthesized following the standard procedures for preparing GS-381 to provide the desired product (5.8 mg, 19% yield) as a light-yellow solid. MS (ESI) m/z=650.4 [M+H]⁺.

### Example 342. 2-(2,6-Dioxopiperidin-3-yl)-5-((6-(4-(8-(piperazin-1-yl)quinoxalin-2-yl)-1H-pyrazol-1-yl)hexyl)amino)isoindoline-1,3-dione (GS-384)

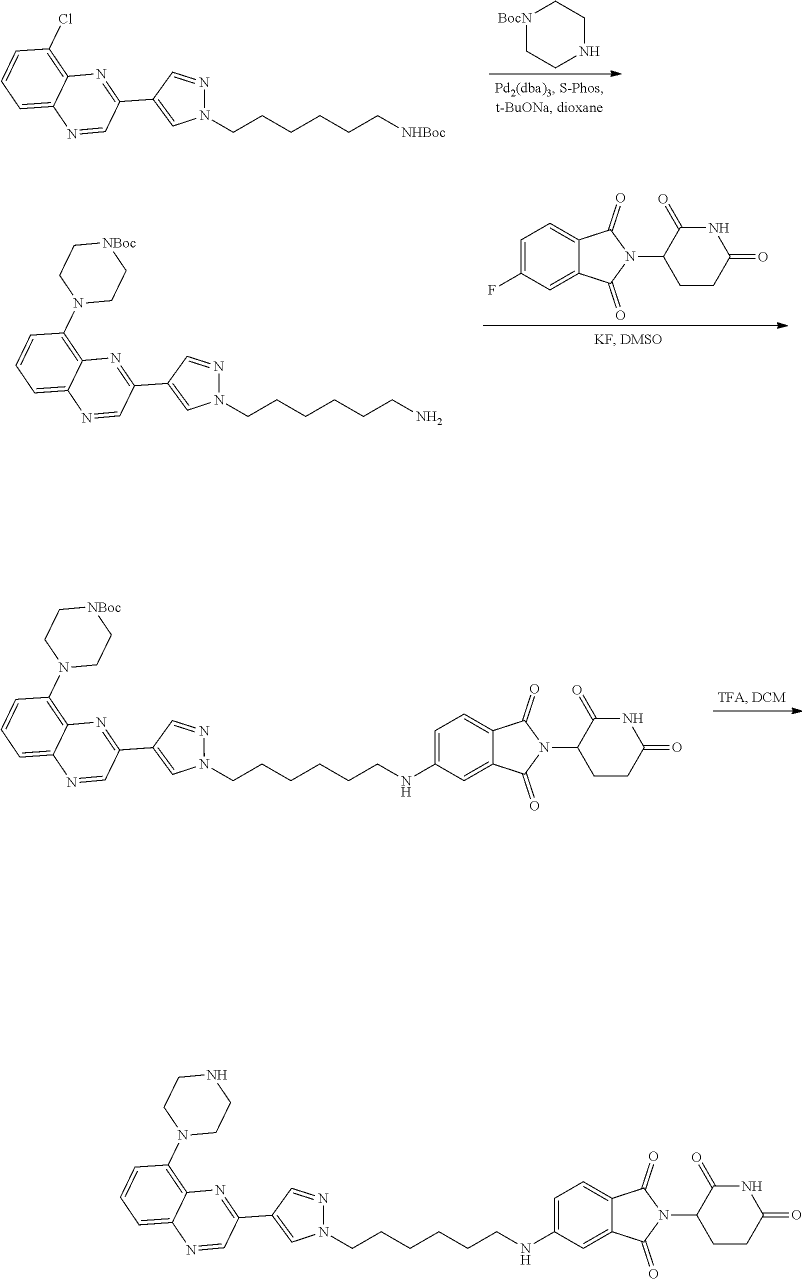 [SMILES: CC(C)(C)OC(=O)NCCCCCCn1cc(-c2cnc3cccc(Cl)c3n2)cn1 ; CC(C)(C)OC(=O)N1CCNCC1 ; CC(C)(C)OC(=O)N1CCN(c2cccc3ncc(-c4cnn(CCCCCCN)c4)nc23)CC1 ; O=C1CCC(N2C(=O)c3ccc(F)cc3C2=O)C(=O)N1 ; CC(C)(C)OC(=O)N1CCN(c2cccc3ncc(-c4cnn(CCCCCCNc5ccc6c(c5)C(=O)N(C5CCC(=O)NC5=O)C6=O)c4)nc23)CC1 ; O=C1CCC(N2C(=O)c3ccc(NCCCCCCn4cc(-c5cnc6cccc(N7CCNCC7)c6n5)cn4)cc3C2=O)C(=O)N1]

GS-384 was synthesized following the procedures for preparing GS-381 (6.4 mg, 71% yield) as a light-yellow solid. MS (ESI) m/z=636.4 [M+H]⁺.

### Example 343. 2-(2,6-Dioxopiperidin-3-yl)-5-((6-((5-(quinoxalin-2-yl)thiazol-2-yl)amino)hexyl)amino)isoindoline-1,3-dione (GS-385)

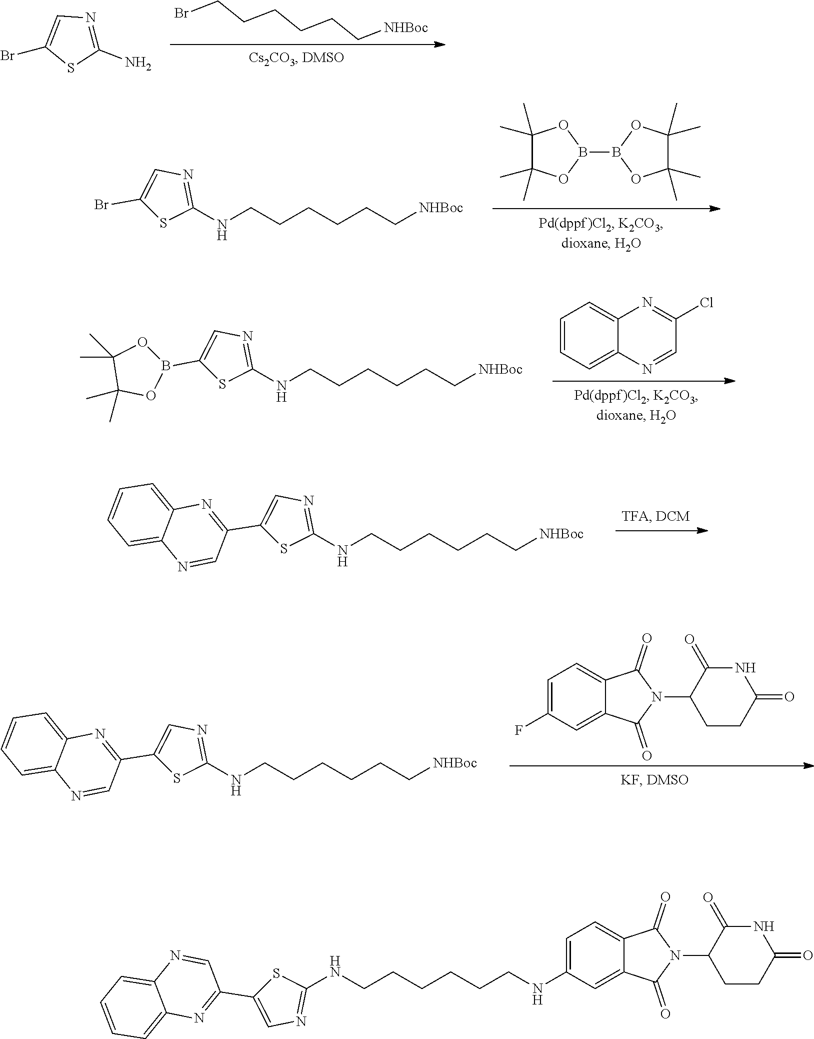

GS-385 was synthesized following the standard procedures for preparing GS-380 (7.5 mg, 51% yield) as a light-yellow solid. MS (ESI) m/z=584.4 [M+H]⁺.

### Example 344. 2-(2,6-Dioxopiperidin-3-yl)-5-((6-(5-(quinoxalin-2-yl)thiazol-2-yl)hexyl)amino)isoindoline-1,3-dione (GS-386)

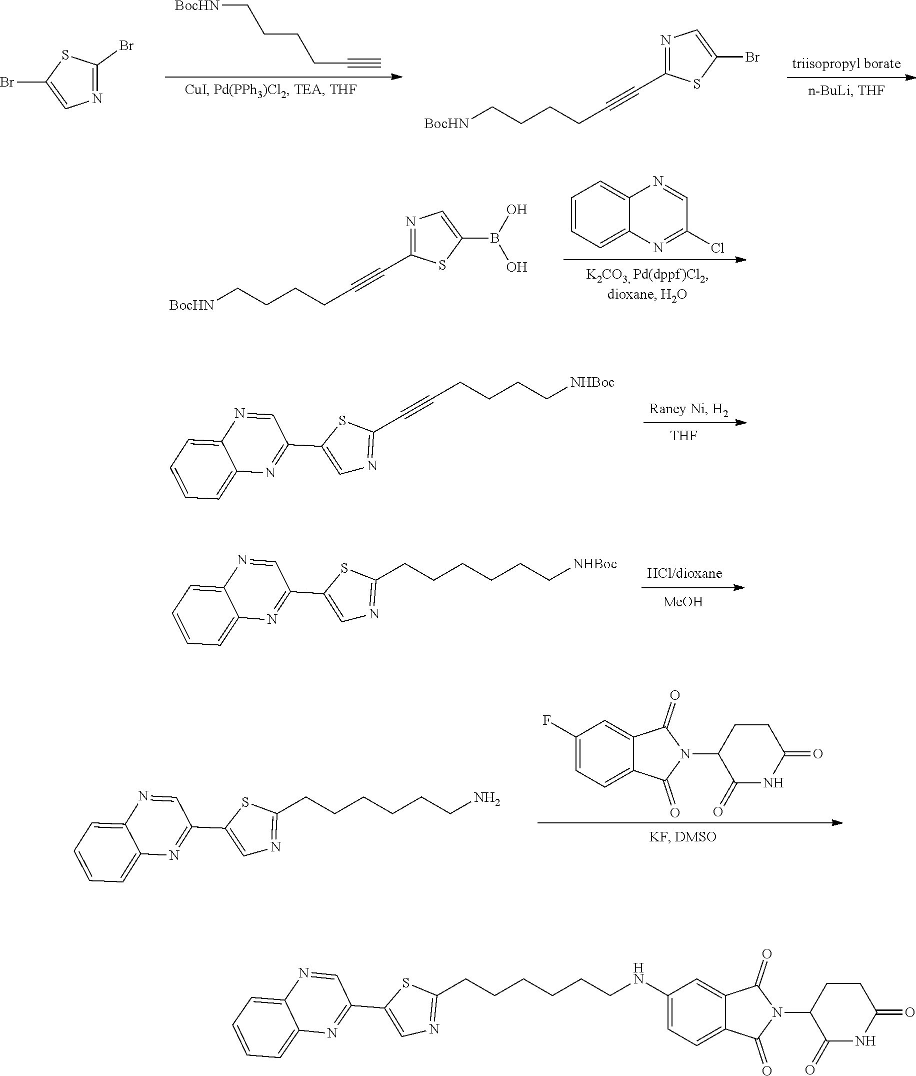

Step 1. Synthesis of tert-butyl (6-(5-bromothiazol-2-yl)hex-5-yn-1-yl)carbamate

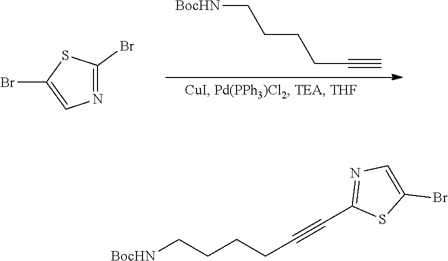 [SMILES: Brc1cnc(Br)s1 ; C#CCCCCNC(=O)OC(C)(C)C ; CC(C)(C)OC(=O)NCCCCC#Cc1ncc(Br)s1]

To a solution of 2,5-dibromothiazole (350 mg, 1.46 mmol) and tert-butyl hex-5-yn-1-ylcarbamate (430 mg, 2.19 mmol) in THF (20 mL) were added TEA (442 mg, 4.38 mmol), Pd(PPh₃)₂Cl₂ (105 mg, 0.15 mmol) and CuI (28 mg, 0.15 mmol). The reaction mixture was stirred at 70° C. under N₂ for 4 h. Then the solution was concentrated and purified by reverse-phase chromatography to provide the desired product (150 mg, 29% yield) as a brown solid. MS (ESI) m/z=359.1 [M+H]⁺.

Step 2. Synthesis of (2-(6-((tert-butoxycarbonyl)amino)hex-1-yn-1-yl)thiazol-5-yl)boronic acid

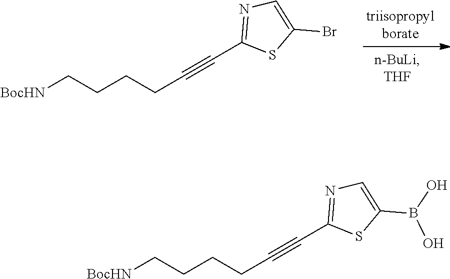 [SMILES: CC(C)(C)OC(=O)NCCCCC#Cc1ncc(Br)s1 ; CC(C)(C)OC(=O)NCCCCC#Cc1ncc(B(O)O)s1]

To a solution of tert-butyl (6-(5-bromothiazol-2-yl)hex-5-yn-1-yl)carbamate (10 mg, 0.42 mmol) in THF (20 mL) was added n-BuLi (0.7 mL, 0.84 mmol) slowly at −78° C. under N₂. After stirring at the same temperature for 30 min, triisopropyl borate (28 mg, 0.15 mmol) was added dropwise. The reaction mixture was stirred at −78° C. for 0.5 h, before it was warmed to rt for another 2 h. The solution was quenched with aqueous NH₄Cl at 0° C. and extracted with ethyl acetate (3×20 mL). The combined organic layers were washed with brine, dried over anhydrous Na₂SO₄, filtered, and concentrated under reduced pressure. The resulting residue was purified by reverse-phase chromatography to provide the desired product (40 mg, 29% yield) as a yellow oil. MS (ESI) m/z=325.2 [M+H]⁺.

Step 3. Synthesis of tert-butyl (6-(5-(quinoxalin-2-yl)thiazol-2-yl)hex-5-yn-1-yl)carbamate

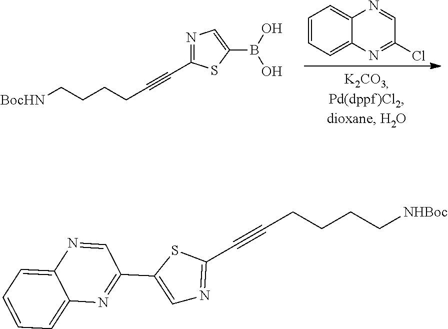 [SMILES: CC(C)(C)OC(=O)NCCCCC#Cc1ncc(B(O)O)s1 ; Clc1cnc2ccccc2n1 ; CC(C)(C)OC(=O)NCCCCC#Cc1ncc(-c2cnc3ccccc3n2)s1]

To a solution of (2-(6-((tert-butoxycarbonyl)amino)hex-1-yn-1-yl)thiazol-5-yl)boronic acid (25 mg, 0.077 mmol) in dioxane (4 mL) and H₂O (1 mL) were added 2-chloroquinoxaline (13 mg, 0.077 mmol), K₂CO₃ (32 mg, 0.23 mmol) and Pd(dppf)Cl₂ (6 mg, 0.008 mmol) at rt under N₂. The reaction mixture was stirred at 80° C. overnight. After cooling down to rt, the mixture was concentrated and purified by reverse-phase chromatography to provide the desired product (10 mg, 39% yield) as a brown solid. MS (ESI) m/z=409.2 [M+H]⁺.

Step 4. Synthesis of tert-butyl (6-(5-(quinoxalin-2-yl)thiazol-2-yl)hexyl)carbamate

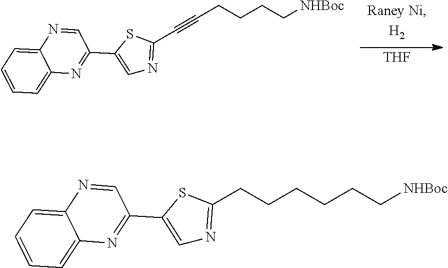 [SMILES: CC(C)(C)OC(=O)NCCCCC#Cc1ncc(-c2cnc3ccccc3n2)s1 ; CC(C)(C)OC(=O)NCCCCCCc1ncc(-c2cnc3ccccc3n2)s1 ; "THF"]

A solution of tert-butyl (6-(5-(quinoxalin-2-yl)thiazol-2-yl)hex-5-yn-1-yl)carbamate (10 mg, 0.024 mmol) and Raney Ni (10 mg) in THF (5 mL) was stirred at rt for 4 h under hydrogen balloon. Then the solution was filtered through Celite and the filtrate was evaporated under reduced pressure. The resulting residue was purified by reverse-phase chromatography to provide the desired product (8 mg, 80% yield) as a light-yellow solid. MS (ESI) m/z=413.4 [M+H]⁺.

Step 5. Synthesis of 6-(5-(quinoxalin-2-yl)thiazol-2-yl)hexan-1-amine

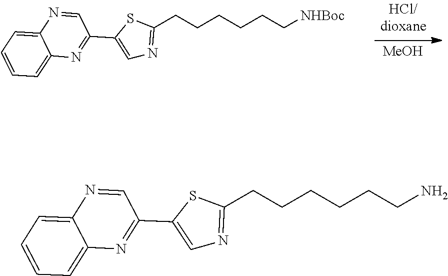 [SMILES: "MeOH" ; CC(C)(C)OC(=O)NCCCCCCc1ncc(-c2cnc3ccccc3n2)s1 ; NCCCCCCc1ncc(-c2cnc3ccccc3n2)s1]

To a solution of tert-butyl (6-(5-(quinoxalin-2-yl)thiazol-2-yl)hexyl)carbamate (8 mg, 0.019 mmol) in MeOH (2 mL) was added HCl solution (4M in dioxane, 2 mL). The reaction mixture was stirred at rt for 2 h, before it was concentrated to provide the crude product (6 mg) as a brown solid. This compound was used in the next step directly without further purification. MS (ESI) m/z=313.2 [M+H]⁺.

Step 6. Synthesis of 2-(2,6-dioxopiperidin-3-yl)-5-((6-(5-(quinoxalin-2-yl)thiazol-2-yl)hexyl)amino) isoindoline-1,3-dione

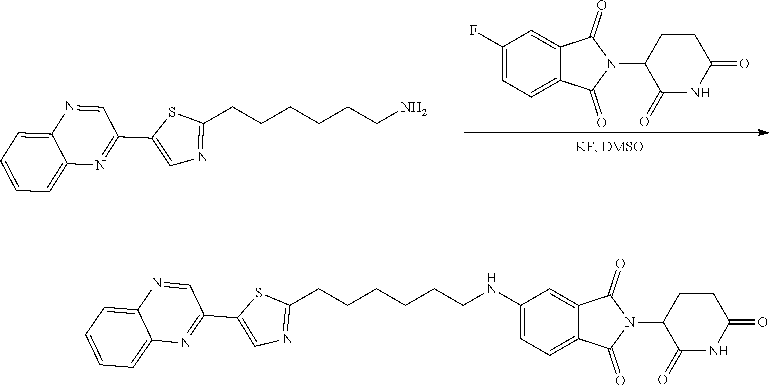 [SMILES: NCCCCCCc1ncc(-c2cnc3ccccc3n2)s1 ; O=C1CCC(N2C(=O)c3ccc(F)cc3C2=O)C(=O)N1 ; O=C1CCC(N2C(=O)c3ccc(NCCCCCCc4ncc(-c5cnc6ccccc6n5)s4)cc3C2=O)C(=O)N1]

To a mixture of 6-(5-(quinoxalin-2-yl)thiazol-2-yl)hexan-1-amine (6 mg, 0.019 mmol) and 2-(2,6-dioxopiperidin-3-yl)-5-fluoroisoindoline-1,3-dione (11 rmg, 0.038 mmol) in DMSO (1 mL) was added KF (6 mg, 0.095 mmol). The reaction mixture was stirred at 140° C. under microwave irradiation for 1 h. After cooling down to rt, the mixture was purified by reverse-phase chromatography to provide the desired product (2 mg, 18% yield) as a yellow solid. MS (ESI) m/z=569.2 [M+H]⁺.

### Example 345. 2-(2,6-Dioxopiperidin-3-yl)-5-((6-(4-(7-methoxyquinoxalin-2-yl)-1H-pyrazol-1-yl)hexyl)amino)isoindoline-1,3-dione (GS-387)

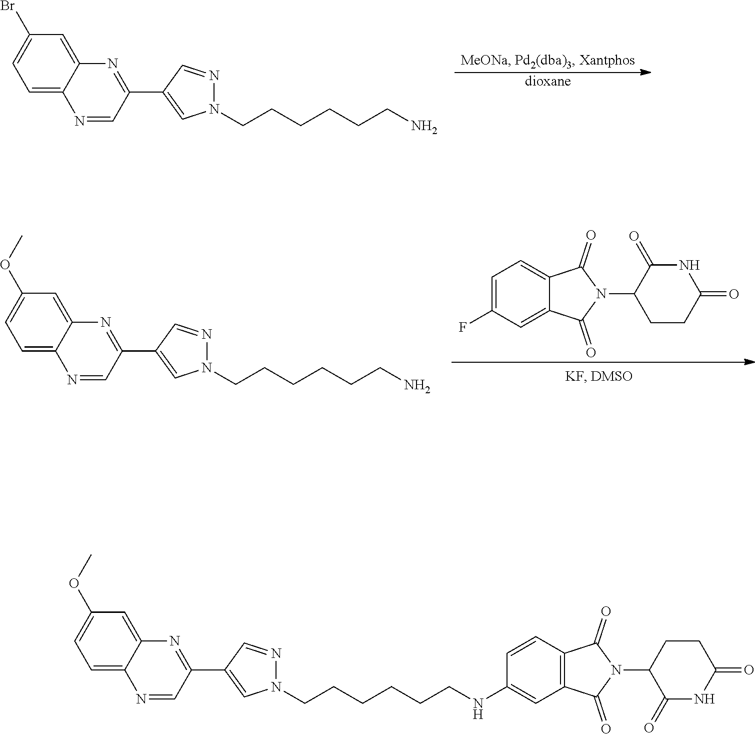

Step 1. Synthesis of 6-(4-(7-methoxyquinoxalin-2-yl)-1H-pyrazol-1-yl)hexan-1-amine

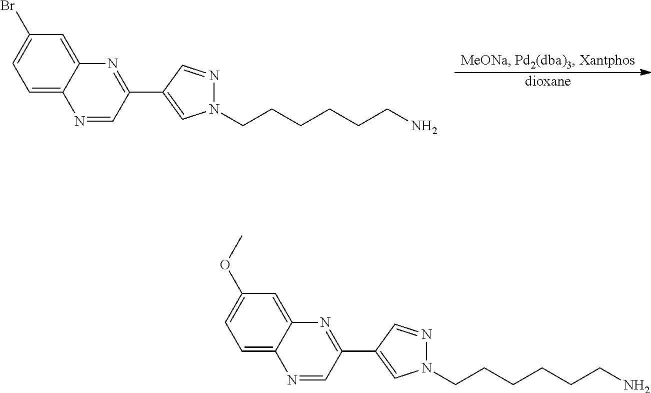 [SMILES: NCCCCCCn1cc(-c2cnc3ccc(Br)cc3n2)cn1 ; "MeONa, Pd2(dba)3, Xantphos" ; "dioxane" ; COc1ccc2ncc(-c3cnn(CCCCCCN)c3)nc2c1]

To a solution of 6-(4-(7-bromoquinoxalin-2-yl)-1H-pyrazol-1-yl)hexan-1-amine (100 mg, 0.27 mmol) in dioxane (10 mL) were added MeONa (43.3 mg, 0.80 mmol), Pd₂(dba)₃ (24.5 mg, 0.03 mmol) and X-Phos (11.0 mg, 0.03 mmol) at rt. Then the mixture was stirred at 100° C. under inert atmosphere for 1 h. After cooling down to rt, the mixture was filtered. The filtrate was concentrated and purified by reverse-phase chromatography to provide the desired product (17.5 mg, 20% yield) as a light-yellow solid. MS (ESI) m/z=326.2 [M+H]⁺.

The second step followed the standard procedure for preparing GS-326 to provide the desired product (3.9 mg, 15% yield) as a light-yellow solid. MS (ESI) m/z=582.4 [M+H]⁺.

### Example 346. 2-(2,6-Dioxopiperidin-3-yl)-5-((6-(4-(8-(1-methylpiperidin-4-yl)quinoxalin-2-yl)-1H-pyrazol-1-yl)hexyl)amino)isoindoline-1,3-dione (GS-388)

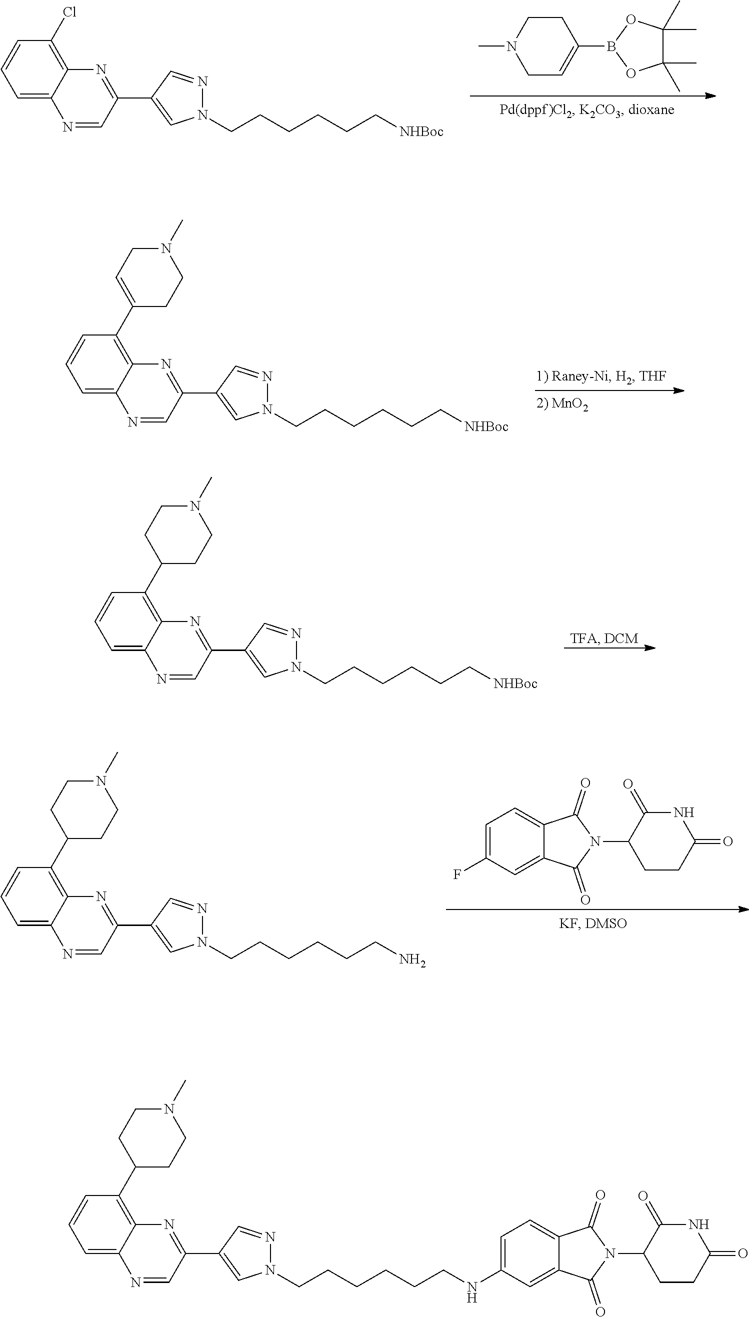

GS-388 was synthesized following the standard procedures for preparing GS-382 (3.4 mg, 20% yield) as a light-yellow solid. MS (ESI) m/z=649.5 [M+H]⁺.

### Example 347. 5-((6-(4-(8-Cyclopropylquinoxalin-2-yl)-1H-pyrazol-1-yl)hexyl)amino)-2-(2,6-dioxopiperidin-3-yl)isoindoline-1,3-dione (GS-389)

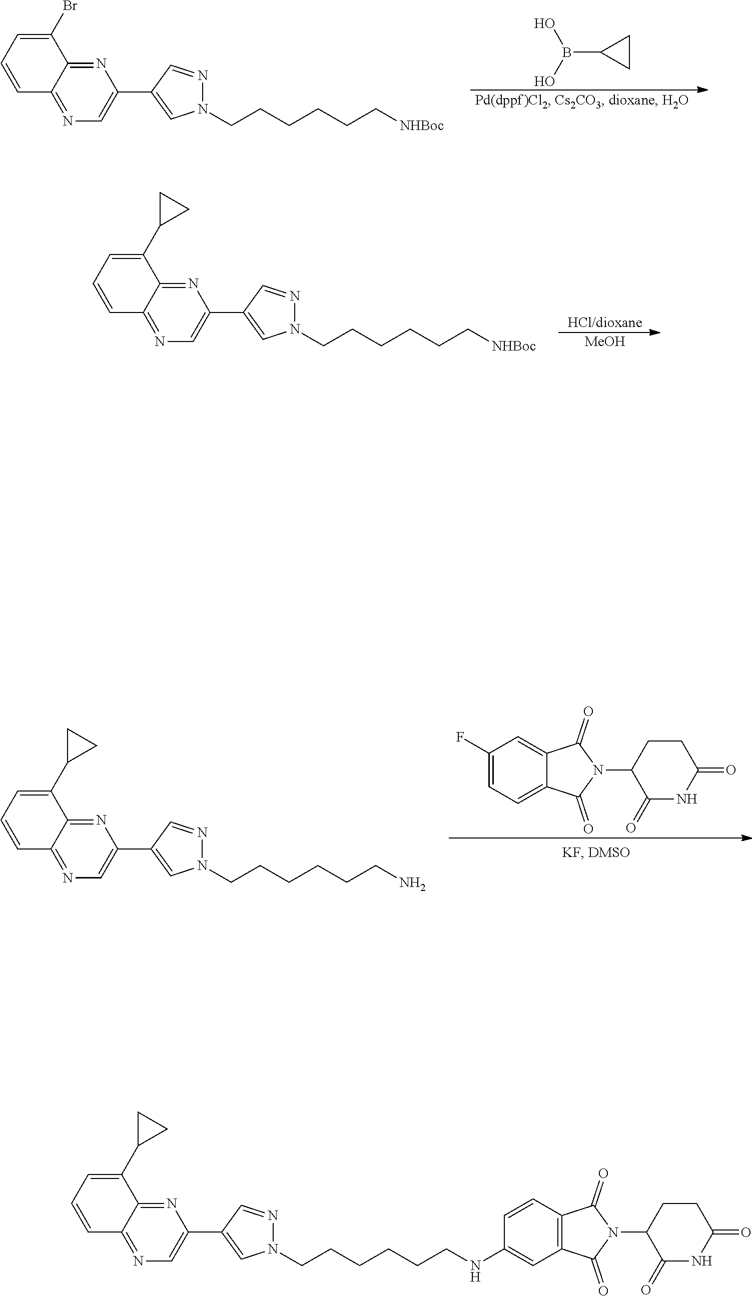

Step 1. Synthesis of tert-butyl (6-(4-(8-cyclopropylqumoxalm-2-yl)-1H-pyrazol-1-yl)hexyl)carbamate

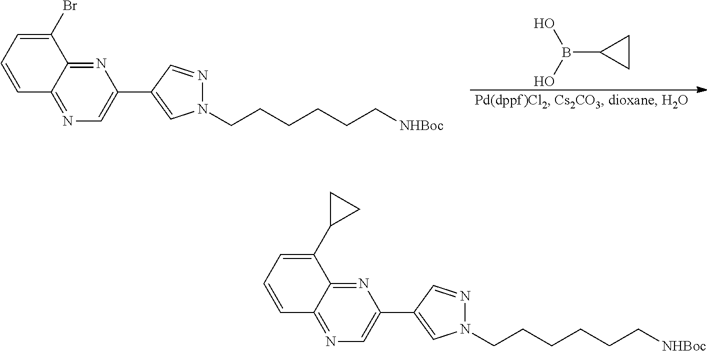

To a solution of tert-butyl (6-(4-(8-bromoquinoxalin-2-yl)-1H-pyrazol-1-yl)hexyl)carbamate (20 mg, 0.042 mmol) in dioxane (5 mL) and H₂O (1 mL) were added cyclopropylboronic acid (12 mg, 0.13 mmol), Cs₂CO₃ (41 mg, 0.13 mmol) and Pd(dppf)Cl₂ (6 mg, 0.008 mmol) at rt under N₂. The reaction mixture was heated at 90° C. for 6 h, before it was concentrated and purified by reverse-phase chromatography to provide the desired product (10 mg, 54% yield) as a brown solid. MS (ESI) m/z=436.4 [M+H]⁺.

The remaining steps were performed according to the procedures for preparing GS-386 to provide the desired product (2 mg, 14% yield) as a light-yellow solid. MS (ESI) m/z=592.3 [M+H]⁺.

### Example 348. 2-(2,6-Dioxopiperidin-3-yl)-5-((5-((5-(quinoxalin-2-yl)thiazol-2-yl)amino)pentyl)amino)isoindoline-1,3-dione (GS-390)

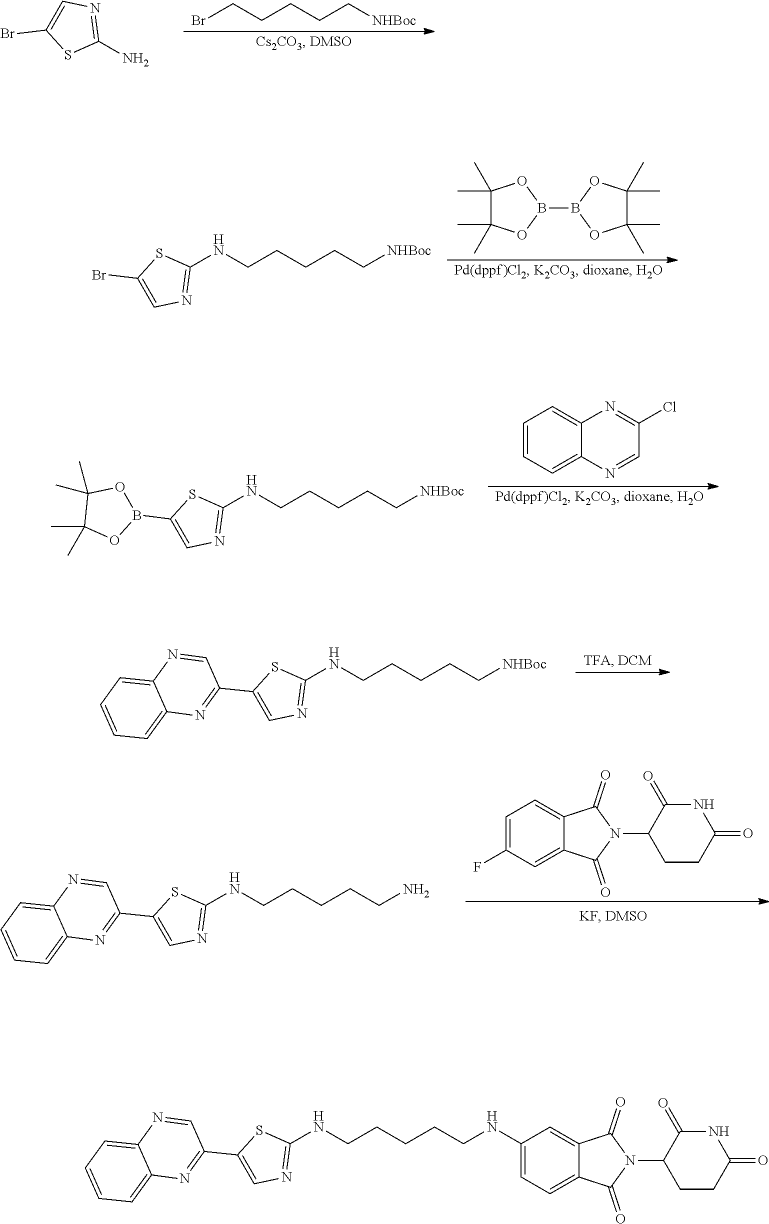

GS-390 was synthesized following the standard procedures for preparing GS-380 (6.4 mg, 38% yield) as a light-yellow solid. MS (ESI) m/z=570.3 [M+H]⁺.

### Example 349. 2-(2,6-Dioxopiperidin-3-yl)-5-((6-(4-(8-methoxyquinoxalin-2-yl)-1H-pyrazol-1-yl)hexyl)amino)isoindoline-1,3-dione (GS-391)

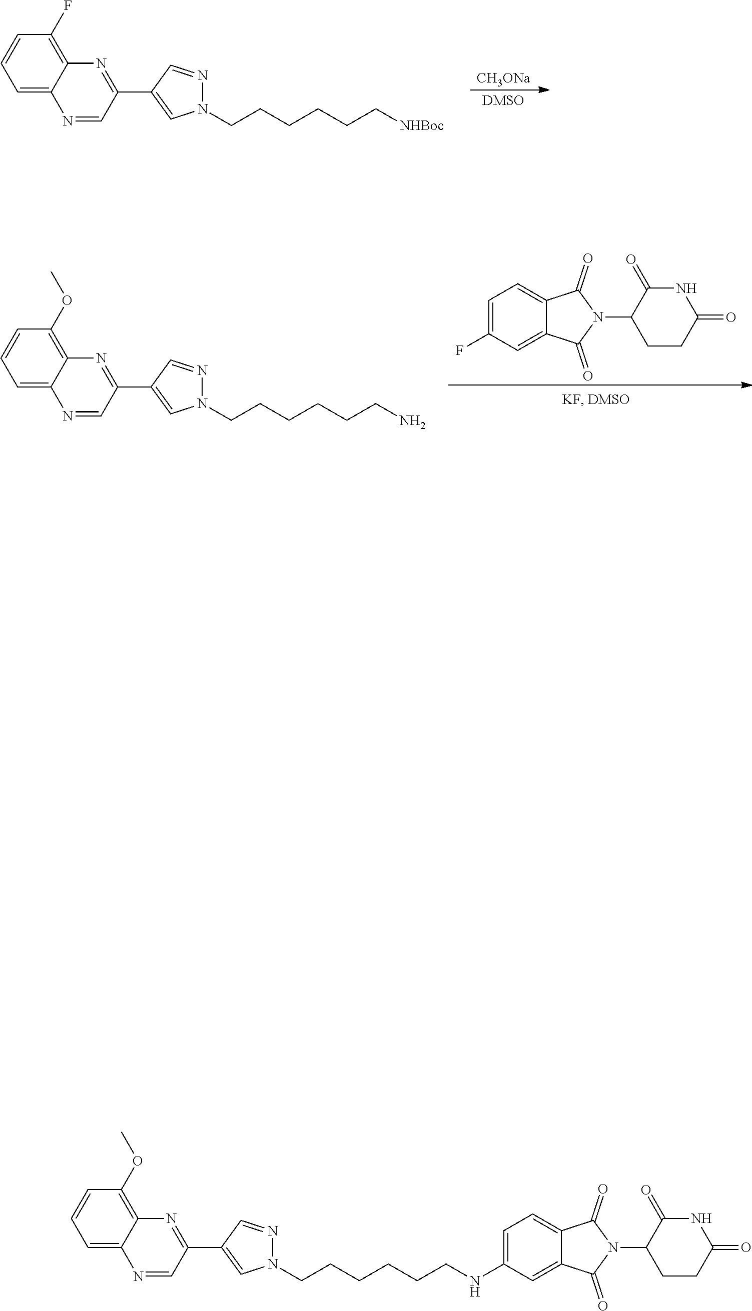

Step 1. Synthesis of 6-(4-(8-methoxyquinoxalin-2-yl)-1H-pyrazol-1-yl)hexan-1-amine

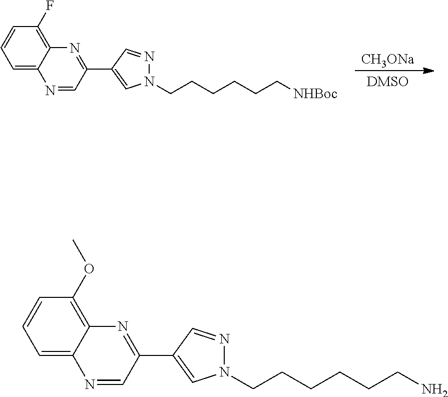

To a solution of tert-butyl (6-(4-(8-fluoroquinoxalin-2-yl)-1H-pyrazol-1-yl)hexyl)carbamate (82 mg, 0.20 mmol) in DMSO (2 mL) was added CH₃ONa (30 mg, 0.60 mmol) at rt. Then the reaction mixture was stirred at 100° C. for 1 h. The mixture was purified by reverse-phase chromatography to provide the desired product (48 mg, 60% yield) as a light-yellow solid. MS (ESI) m/z=326.2 [M+H]⁺.

The remaining step was performed according to the standard procedure for preparing GS-326 (2.6 mg, 31% yield) as a light-yellow solid. MS (ESI) m/z=582.3 [M+H]⁺.

### Example 350. 2-(2,6-Dioxopiperidin-3-yl)-5-(((3-(4-(8-morpholinoquinoxalin-2-yl)-1H-pyrazol-1-yl)cyclobutyl)methyl)amino)isoindoline-1,3-dione (GS-392)

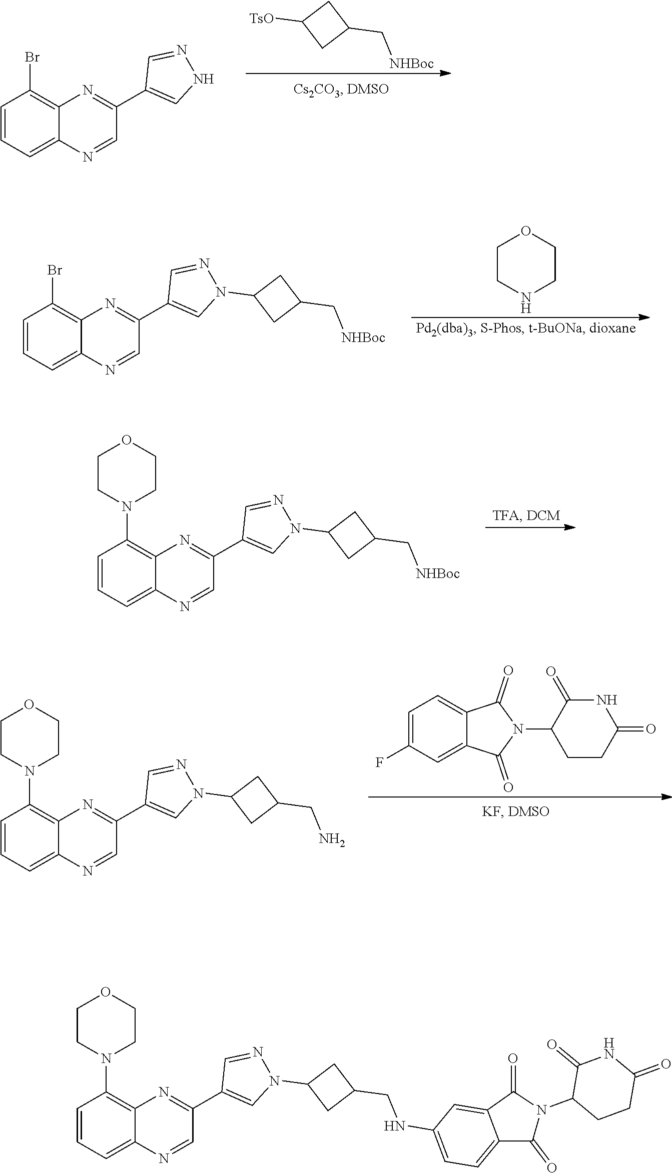

GS-392 was synthesized following the standard procedures for preparing GS-381 (1.7 mg, 15% yield) as a light-yellow solid. MS (ESI) m/z=621.5 [M+H]⁺.

### Example 351. 2-(2,6-Dioxopiperidin-3-yl)-5-((6-(4-(8-(piperidin-4-yl)quinoxalin-2-yl)-1H-pyrazol-1-yl)hexyl)amino)isoindoline-1,3-dione (GS-393)

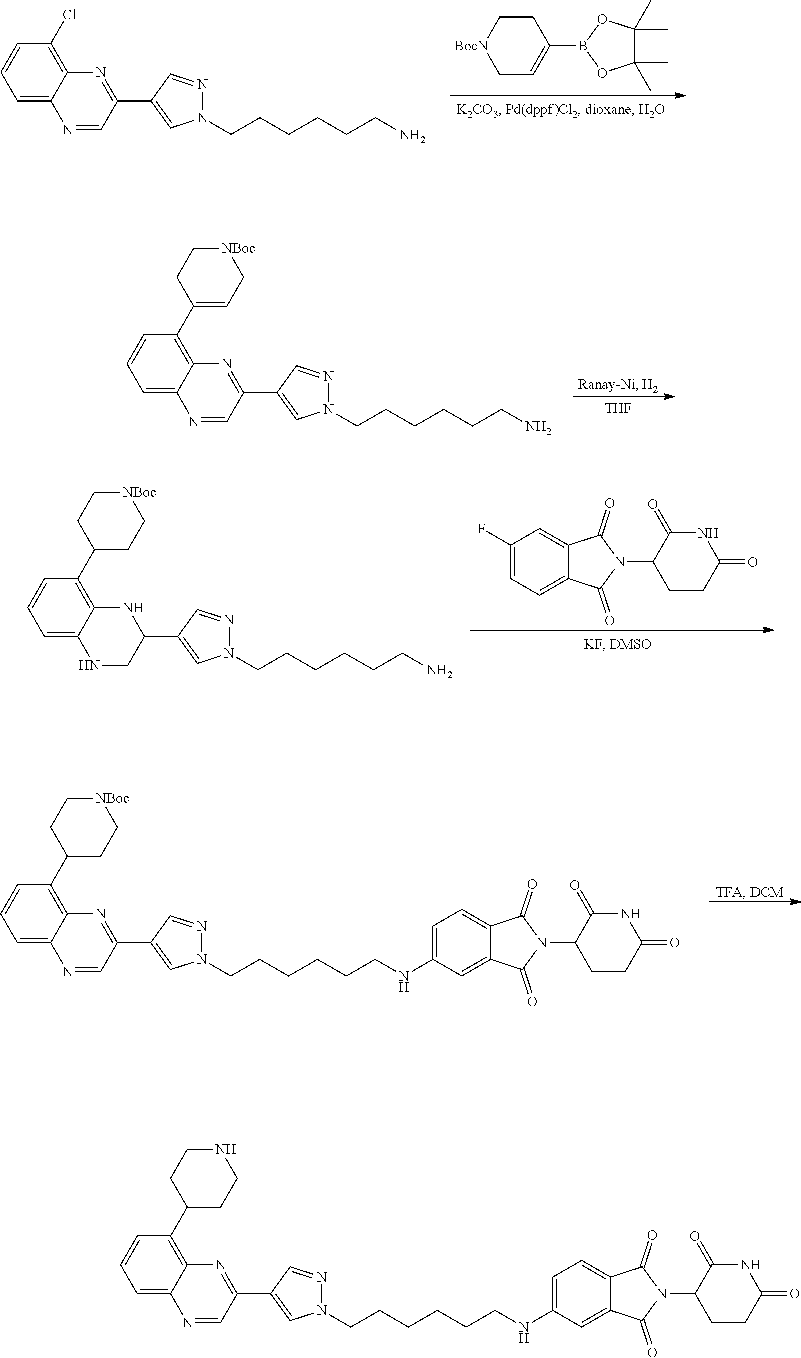

GS-393 was synthesized following the standard procedures for preparing GS-382 (5.2 mg, 32% yield) as a light-yellow solid. MS (ESI) m/z=635.3 [M+H]⁺.

### Example 352. 2-(2,6-Dioxopiperidin-3-yl)-5-(((3-(4-(8-fluoroquinoxalin-2-yl)-1H-pyrazol-1-yl)cyclobutyl)methyl)amino)isoindoline-1,3-dione (GS-394)

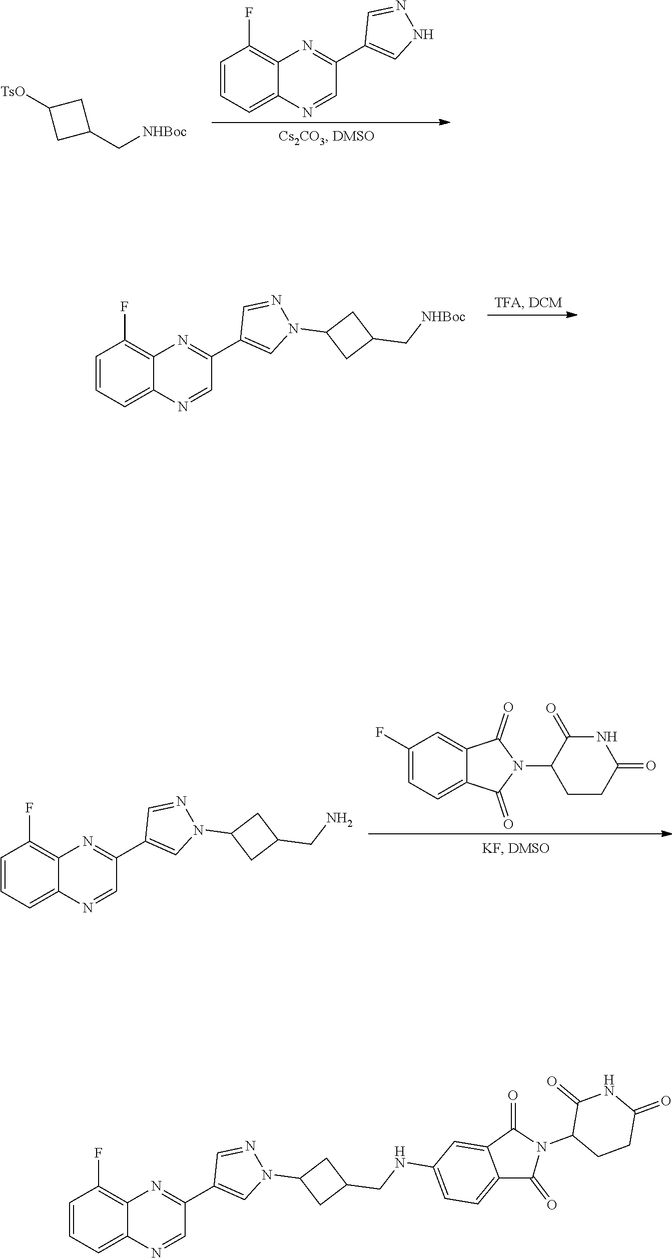

Step 1. Synthesis of tert-butyl ((3-(4-(8-fluoroquinoxalin-2-yl)-1H-pyrazol-1-yl)cyclobutyl)methyl)carbamate

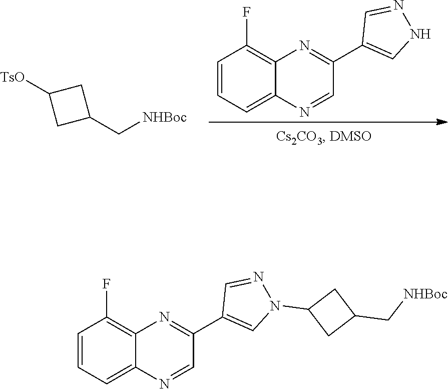

To a solution of 3-(((tert-butoxycarbonyl)amino)methyl)cyclobutyl 4-methylbenzenesulfonate (70 mg, 0.20 mmol) and 8-fluoro-2-(1H-pyrazol-4-yl)quinoxaline (23 mg, 0.20 mmol) in DMSO (3 mL) was added Cs₂CO₃ (130 mg, 0.40 mmol) at rt. Then the reaction mixture was stirred at 100° C. for 3 h. The mixture was purified by reverse-phase chromatography to provide the desired product (48 mg, 60% yield) as a light-yellow solid. MS (ESI) m/z=398.2 [M+H]⁺.

The remaining steps were performed according to the standard procedures for preparing GS-326 to provide the desired product (3.8 mg, 30% yield) as a light-yellow solid. MS (ESI) m/z=554.2 [M+H]⁺.

### Example 353. 2-(2,6-Dioxopiperidin-3-yl)-5-((3-(3-(4-(7-((1R,4R)-5-methyl-2,5-diazabicyclo[2.2.1]heptan-2-yl)quinoxalin-2-yl)-1H-pyrazol-1-yl)cyclobutyl)propyl)amino)isoindoline-1,3-dione (GS-395)

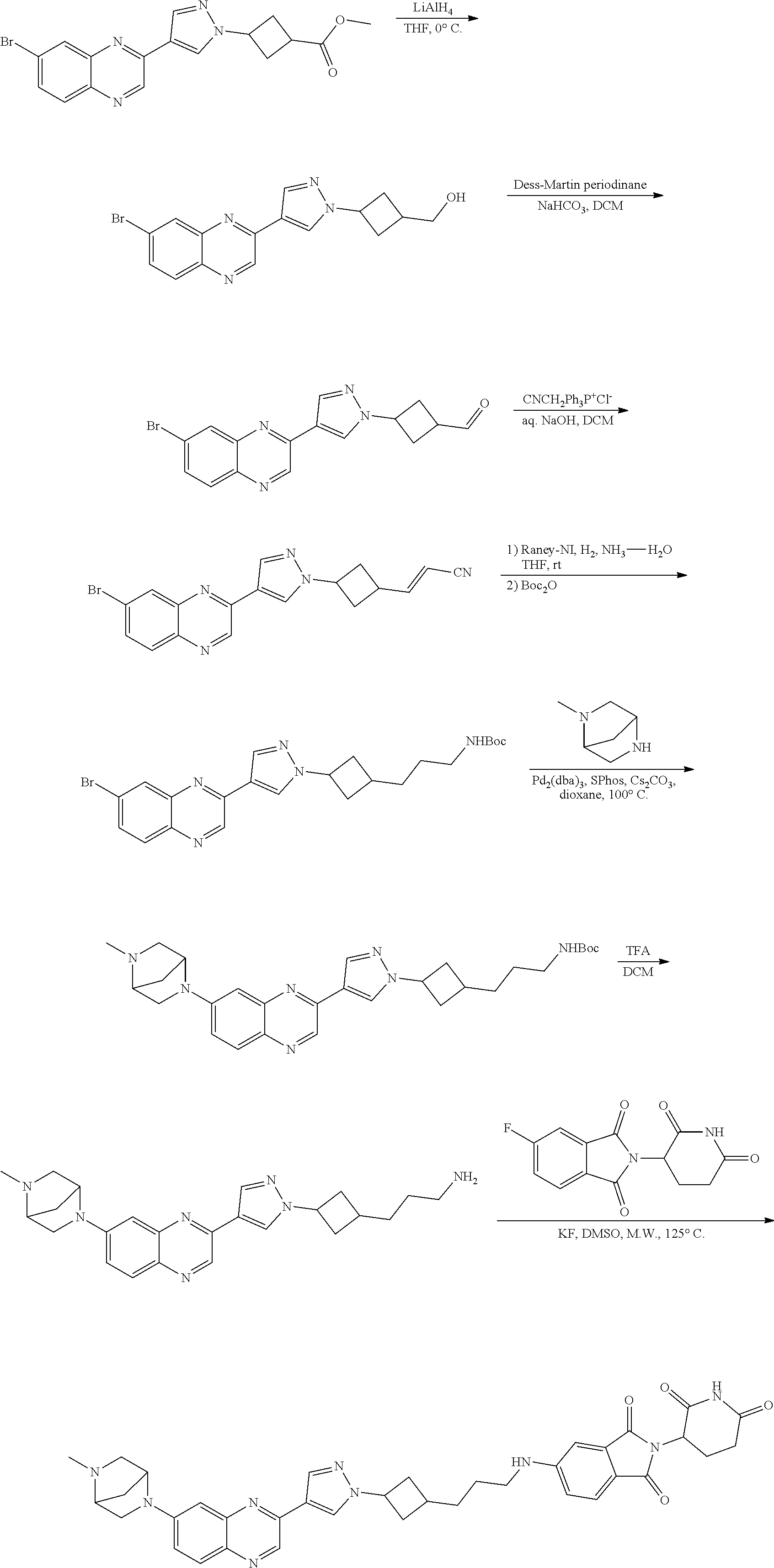

Step 1. Synthesis of (3-(4-(7-bromoquinoxalin-2-yl)-1H-pyrazol-1-yl)cyclobut yl)methanol

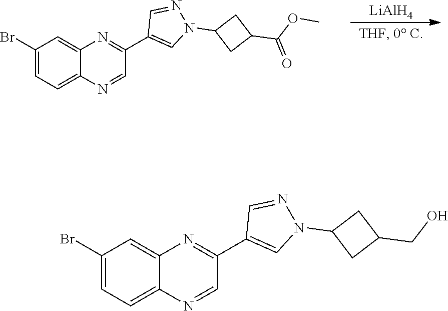 [SMILES: COC(=O)C1CC(n2cc(-c3cnc4ccc(Br)cc4n3)cn2)C1 ; "LiAlH4" ; "THF, 0° C." ; OCC1CC(n2cc(-c3cnc4ccc(Br)cc4n3)cn2)C1]

To a solution of methyl 3-(4-(7-bromoquinoxalin-2-yl)-1H-pyrazol-1-yl)cyclobutane-1-carboxylate (350.0 mg, 0.90 mmol) in THF (8.0 mL) was added LiAlH₄ (67.9 mg, 1.79 mmol) at 0° C. in portions over 10 min. The reaction mixture was stirred at the same temperature for 1 h. Then the reaction was quenched with solid Na₂SO₄ and diluted with EtOAc (10.0 mL). The suspension was filtered through Celite and the filtrate was concentrated to provide the crude product (300 mg, 92% yield) as a yellow solid which was used in the next step directly. MS (ESI) m/z=359.0 [M+H]⁺.

Step 2. Synthesis of 3-(4-(7-bromoquinoxalin-2-yl)-1H-pyrazol-1-yl)cyclobutane-1-carb aldehyde

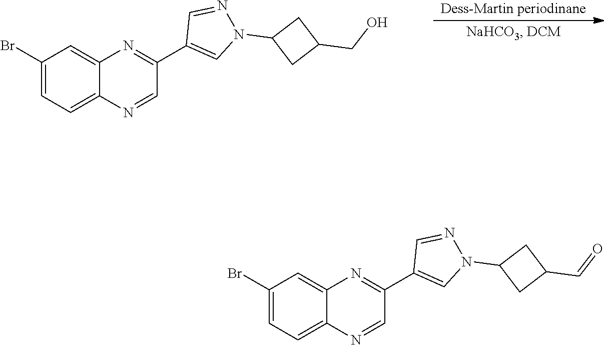

To a suspension of (3-(4-(7-bromoquinoxalin-2-yl)-1H-pyrazol-1-yl)cyclobutyl)methanol (300 mg crude, 0.84 mmol) and NaHCO₃ (141.1 mg, 1.68 mmol) in DCM (10.0 mL) was added Dess-Martin Periodinane (534.2 mg, 1.26 mmol) at 0° C. in 3 portions. Then the solution was stirred at rt for 2 h. The reaction mixture was diluted with DCM (10.0 mL) and filtered through Celite. The filtrate was concentrated to provide the crude product (300 mg, 100% yield) as a yellow solid which was used in the next step directly. MS (ESI) m/z=357.0 [M+H]⁺.

Step 3. Synthesis of (E)-3-(3-(4-(7-bromoquinoxalin-2-yl)-1H-pyrazol-1-yl)cyclobutyl) acrylonitrile

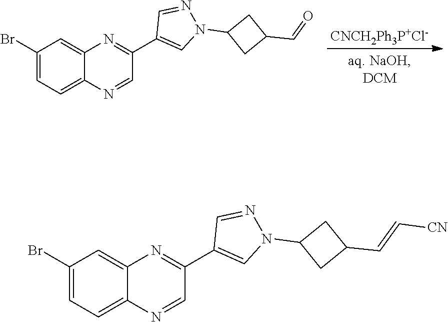 [SMILES: O=CC1CC(n2cc(-c3cnc4ccc(Br)cc4n3)cn2)C1 ; N#C/C=C/C1CC(n2cc(-c3cnc4ccc(Br)cc4n3)cn2)C1 ; "CNCH2Ph3P+Cl-"]

To a mixture of (cyanomethyl)triphenylphosphonium chloride (283.1 mg, 0.84 mmol) in DCM (5.0 mL) was added 30% aqueous NaOH solution (0.5 mL) at 0° C. After stirring for 5 min, 3-(4-(7-bromoquinoxalin-2-yl)-1H-pyrazol-1-yl)cyclobutane-1-carbaldehyde (300 mg crude, 0.84 mmol) was added into the reaction mixture and the solution was stirred at the same temperature for another 10 min. Then the reaction was diluted with ice water and extracted with DCM (3×8 mL). The organic layer was dried over anhydrous Na₂SO₄, filtered, and concentrated. The resulting residue was purified by reverse-phase chromatography to provide the desired product (170.0 mg, 50% yield by 3 steps) as a pale yellow solid. MS (ESI) m/z=380.0 [M+H]⁺.

Step 4. Synthesis of tert-butyl (3-(3-(4-(7-bromoquinoxalin-2-yl)-1H-pyrazol-1-yl)cyclobutyl)propyl) carbamate

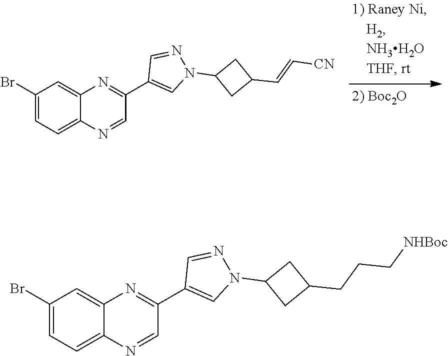 [SMILES: N#C/C=C/C1CC(n2cc(-c3cnc4ccc(Br)cc4n3)cn2)C1 ; CC(C)(C)OC(=O)NCCCC1CC(n2cc(-c3cnc4ccc(Br)cc4n3)cn2)C1]

To a solution of (E)-3-(3-(4-(7-bromoquinoxalin-2-yl)-1H-pyrazol-1-yl)cyclobutyl)acrylo nitrile (170.0 mg, 0.45 mmol) in THF (5.0 mL) and NH₃·H₂O (0.5 mL) was added Raney-Ni catalyst (34.0 mg, 20% wt) at rt. The mixture was purged with hydrogen and stirred at rt for 20 min under hydrogen balloon. Then Boc₂O (96.6 mg, 0.447 mmol) was added into the reaction mixture and it was stirred at rt for another 20 min. The solution was filtered through Celite and the cake was washed with EtOAc (10 mL). The filtrate was concentrated and purified by reverse-phase chromatography to provide the desired product (80.0 mg, 37% yield) as a yellow solid. MS (ESI) m/z=486.2 [M+H]⁺.

Step 5. Synthesis of tert-butyl (3-(3-(4-(7-((1R,4R)-5-methyl-2,5-diazabicyclo[2.2.1]heptan-2-yl)quin oxalin-2-yl)-1H-pyrazol-1-yl)cyclobutyl)propyl)carbamate

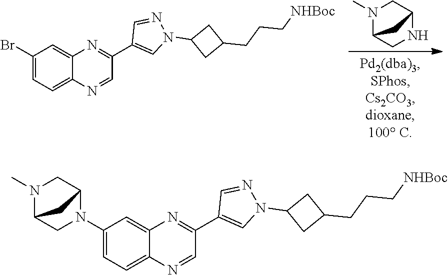

To a suspension of tert-butyl (3-(3-(4-(7-bromoquinoxalin-2-yl)-1H-pyrazol-1-yl)cyclobutyl) propyl)carbamate (24.3 mg, 0.05 mmol) and (1R,4R)-2-methyl-2,5-diazabicyclo[2.2.1]heptane (18.3 mg, 0.10 mmol) in dioxane (2.0 mL) were added Cs₂CO₃ (48.9 mg, 0.15 mmol), S-Phos (4.1 mg, 0.01 mmol) and Pd₂(dba)₃ (4.6 mg, 0.005 mmol) at rt. The reaction mixture was purged with N₂ and stirred at 100° C. for 12 h. After cooling down to rt, the solution was diluted with EtOAc (8.0 mL) and filtered through Celite. The filtrate was concentrated and purified by reverse-phase chromatography to provide the desired product (20.0 mg, 65% yield) as a yellow solid. MS (ESI) m/z=518.5 [M+H]⁺.

Step 6. Synthesis of 3-(3-(4-(7-((1R,4R)-5-methyl-2,5-diazabicyclo[2.2.1]heptan-2-yl)quinoxalin-2-yl)-1H-pyrazol-1-yl)cyclobutyl)propan-1-amine

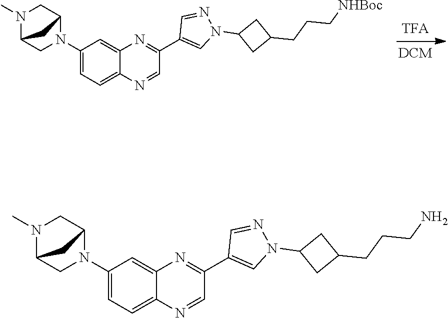 [SMILES: CN1C[C@H]2C[C@@H]1CN2c1ccc2ncc(-c3cnn(C4CC(CCCNC(=O)OC(C)(C)C)C4)c3)nc2c1 ; CN1C[C@H]2C[C@@H]1CN2c1ccc2ncc(-c3cnn(C4CC(CCCN)C4)c3)nc2c1]

To a solution of tert-butyl (3-(3-(4-(7-((1R,4R)-5-methyl-2,5-diazabicyclo[2.2.1]heptan-2-yl)quin oxalin-2-yl)-1H-pyrazol-1-yl)cyclobutyl)propyl)carbamate (20.0 mg, 0.032 mmol) in DCM (0.5 mL) was added TFA (0.5 mL) at rt. The reaction mixture was stirred at rt for 1 h, before it was concentrated under vacuum to provide the crude product (16.5 mg, 100% yield) as a red brown solid which was used in the next step directly. MS (ESI) m/z=418.3 [M+H]⁺.

Step 7. Synthesis of 2-(2,6-dioxopiperidin-3-yl)-5-((3-(3-(4-(7-((1R,4R)-5-methyl-2,5-diazabicyclo [2.2.1]heptan-2-yl)quinoxalin-2-yl)-1H-pyrazol-1-yl)cyclobutyl)propyl)amino)isoindoline-1,3-dione

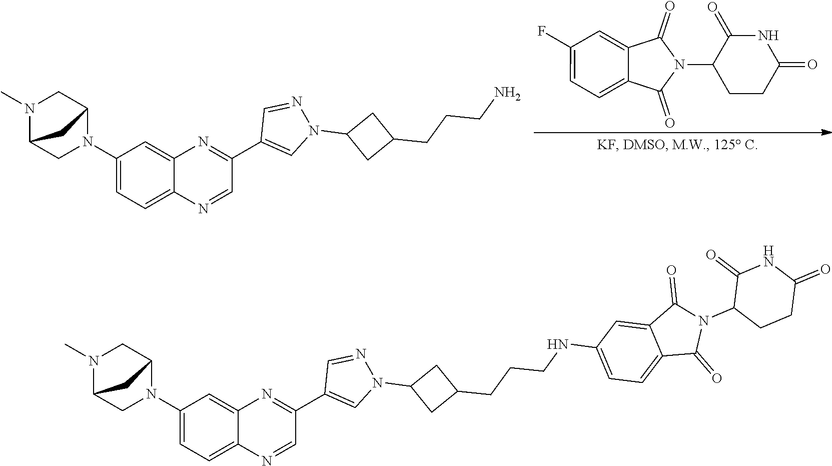 [SMILES: CN1C[C@H]2C[C@@H]1CN2c1ccc2ncc(-c3cnn(C4CC(CCCN)C4)c3)nc2c1 ; O=C1CCC(N2C(=O)c3ccc(F)cc3C2=O)C(=O)N1 ; CN1C[C@H]2C[C@@H]1CN2c1ccc2ncc(-c3cnn(C4CC(CCCNc5ccc6c(c5)C(=O)N(C5CCC(=O)NC5=O)C6=O)C4)c3)nc2c1]

To a mixture of 3-(3-(4-(7-((1R,4R)-5-methyl-2,5-diazabicyclo[2.2.1]heptan-2-yl)quinoxalin-2-yl)-1H-pyrazol-1-yl)cyclobutyl)propan-1-amine (16.5 mg crude, 0.032 mmol) and KF (18.6 mg, 0.32 mmol) in DMSO (0.5 mL) was added 2-(2,6-dioxopiperidin-3-yl)-5-fluoroisoindoline-1,3-dione (17.7 mg, 0.064 mmol) at rt. Then the mixture was stirred at 125° C. under microwave irradiation for 25 min. After cooling down to rt, the mixture was purified by reverse-phase chromatography to provide the desired product (5.3 mg, 25% yield) as a yellow solid. MS (ESI) m/z=674.5 [M+H]⁺.

### Example 354. 2-(2,6-Dioxopiperidin-3-yl)-5-((3-(trans-3-(4-(6-morpholinoquinoxalin-2-yl)-1H-pyrazol-1-yl)cyclobutyl)propyl)amino)isoindoline-1,3-dione (GS-396)

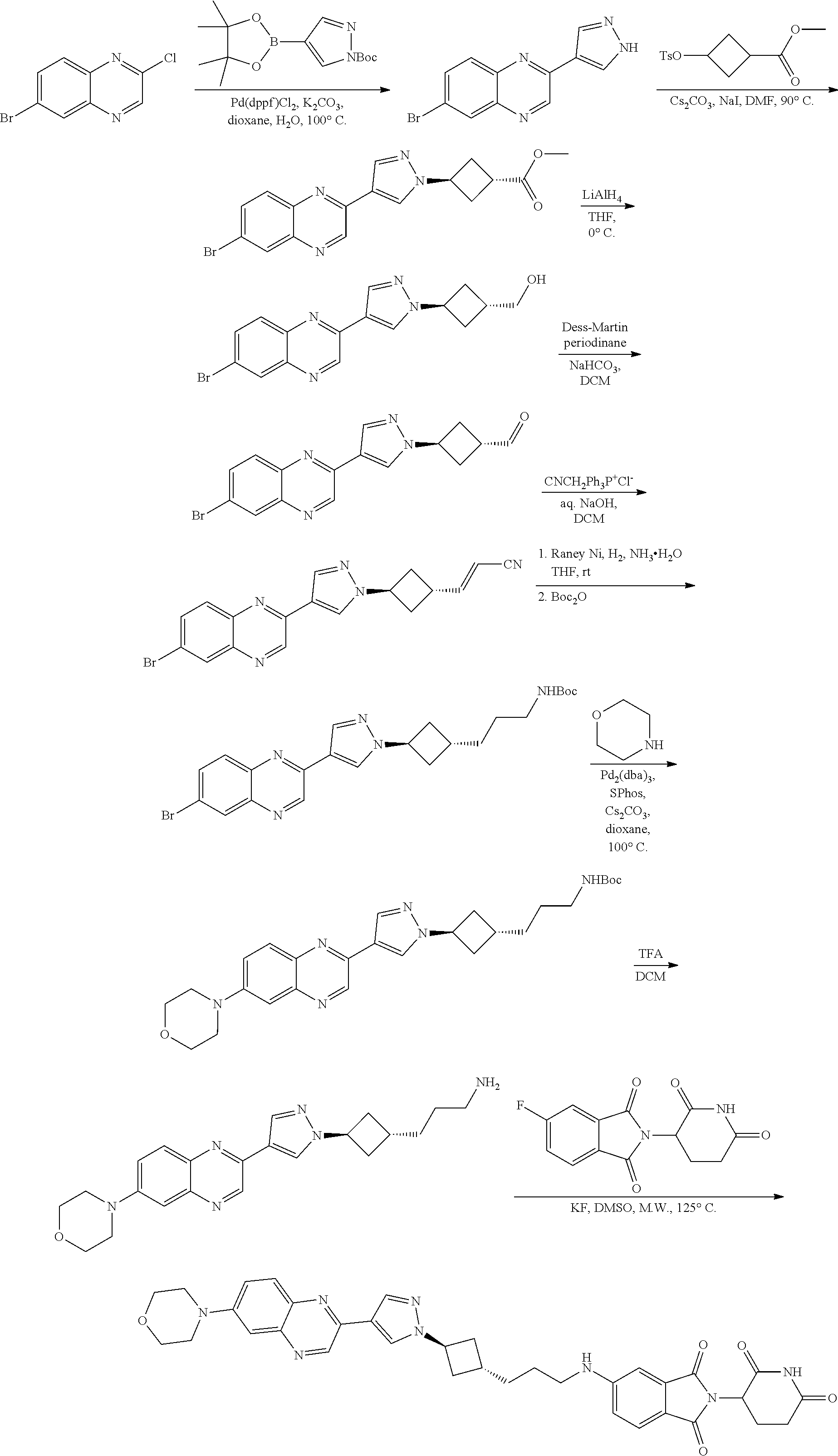

GS-396 was synthesized following the procedures for preparing GS-395 (3 mg, 12% yield) as a red solid. MS (ESI) mzz 649.3 [M+H]⁺.

### Example 355. 2-(2,6-Dioxopiperidin-3-yl)-5-((3-(trans-3-(4-(6-(4-methylpiperazin-1-yl)quinoxalin-2-yl)-1H-pyrazol-1-yl)cyclobutyl)propyl)amino)isoindoline-1,3-dione (GS-397)

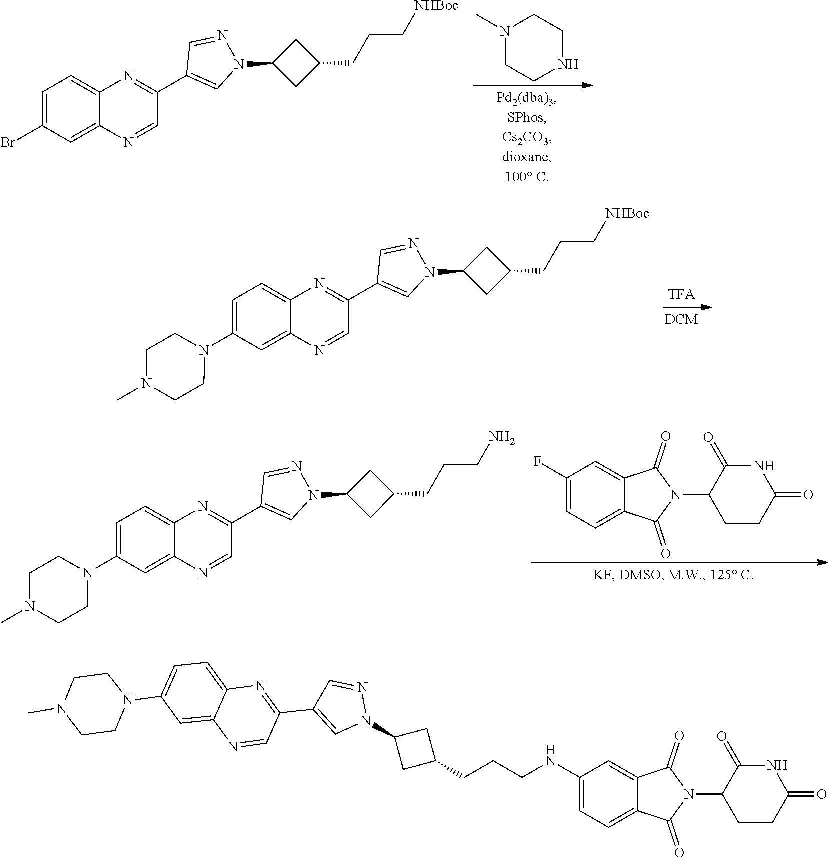

GS-397 was synthesized following the standard procedures for preparing GS-395 (5.0 mg, 12% yield by 3 steps) as a red solid. MS (ESI) m/z=662.5 [M+H]⁺.

### Example 356. 2-(2,6-Dioxopiperidin-3-yl)-5-(((3-(4-(8-(4-methylpiperazin-1-yl)quinoxalin-2-yl)-1H-pyrazol-1-yl)cyclobutyl)methyl)amino)isoindoline-1,3-dione (GS-398)

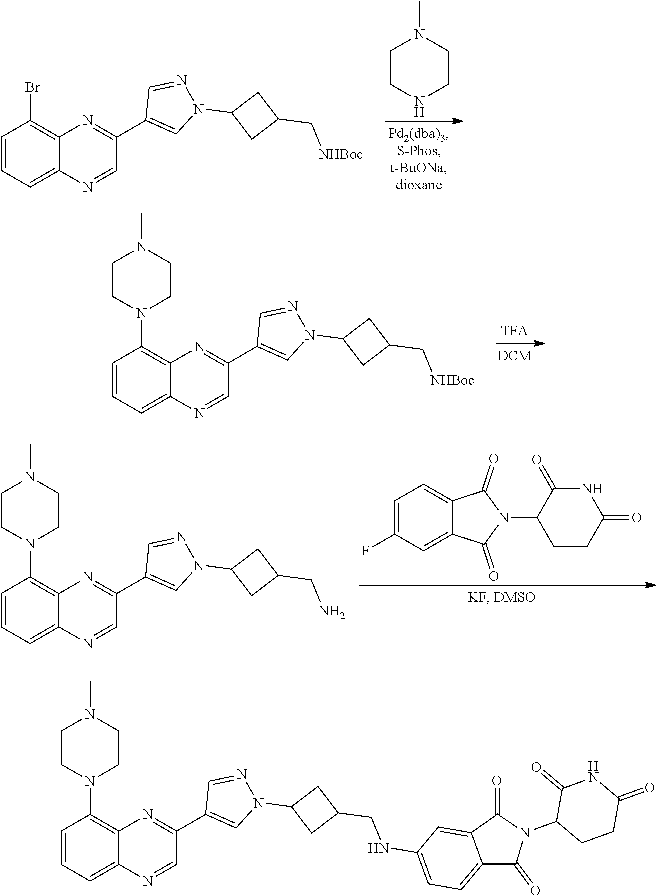

GS-398 was synthesized following the standard procedures for preparing GS-381 (2.5 mg, 19% yield) as a light-yellow solid. MS (ESI) m/z=634.5 [M+H]⁺.

### Example 357. 3-(5-((3-(trans-3-(3-Methyl-4-(7-morpholinoquinoxalin-2-yl)-1H-pyrazol-1-yl)cyclobutyl)propyl)amino)-1,3-dioxo-2,3-dihydro-1H-inden-2-yl)piperidine-2,6-dione (GS-399)

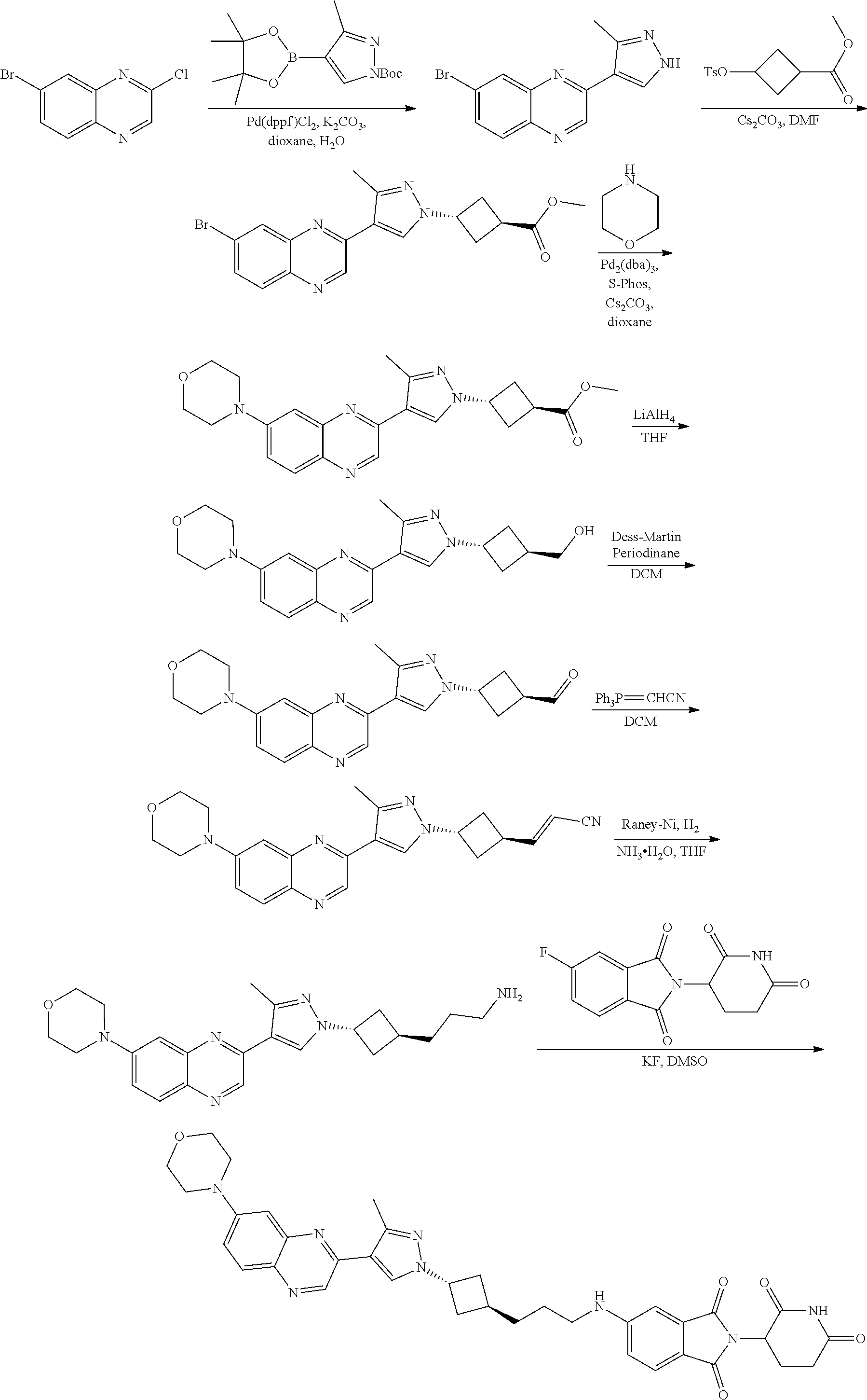

Step 1. Synthesis of 7-bromo-2-(3-methyl-H-pyrazol-4-yl)quinoxaline

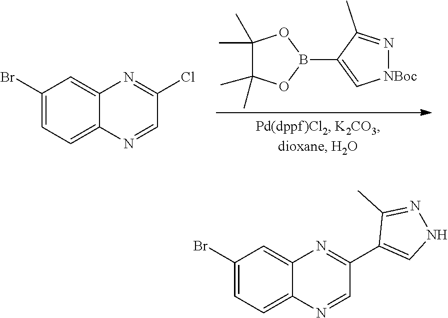

To a solution of 7-bromo-2-chloroquinoxaline (100 mg, 0.4 mmol) in dioxane (10.0 mL) and H₂O (2.0 mL) were added tert-butyl 4-(4,4,5,5-tetramethyl-1,3,2-dioxaborolan-2-yl)-1H-pyrazole-1-carboxylate (144.1 mg, 0.49 mmol), Pd(dppf)Cl₂ (17.9 mg, 0.025 mmol) and K₂CO₃ (113.2 mg, 0.82 mmol) at rt. The mixture was stirred at 100° C. under inert atmosphere for 8 h. After cooling down to rt, the reaction mixture was poured into water (20 mL) and extracted with ethyl acetate (3×20 mL). The combined organic layers were washed with saturated brine (20 mL), dried over anhydrous sodium sulfate, filtered, and concentrated under reduced pressure. The resulting residue was purified by reverse-phase chromatography to provide the desired product (85.0 mg, 72% yield) as a pale white solid. MS (ESI) m/z=289.0 [M+H]⁺.

Step 2. Synthesis of methyl trans-3-(4-(7-bromoquinoxalin-2-yl)-3-methyl-1H-pyrazol-1-yl)cyclobutane-1-carboxylate

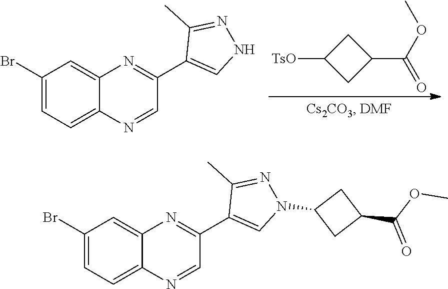 [SMILES: Cc1n[nH]cc1-c1cnc2ccc(Br)cc2n1 ; COC(=O)C1CC(OS(=O)(=O)c2ccc(C)cc2)C1 ; COC(=O)[C@H]1C[C@H](n2cc(-c3cnc4ccc(Br)cc4n3)c(C)n2)C1]

To a solution of 6-bromo-2-(1H-pyrazol-4-yl)quinoxaline (400.0 mg, 1.0 mmol) and methyl 3-(tosyloxy)cyclobutane-1-carboxylate (611 mg, 1.5 mmol) in DMSO (10 mL) were added Cs₂CO₃ (978 mg, 3.0 mmol) and NaI (75 mg, 0.5 mmol) at rt. The reaction mixture was stirred at 90° C. for 2 h. After cooling down to rt, the mixture was poured into water (50 mL) and extracted with ethyl acetate (3×20 mL). The combined organic layers were washed with saturated brine (20 mL), dried over anhydrous sodium sulfate, filtered, and concentrated under reduced pressure. The resulting residue was purified by silica gel column chromatography to provide four isomers. The desired trans-isomer (79.0 mg, 20% yield) was got as a pale white solid. MS (ESI) m/z=401.1 [M+H]⁺.

Step 3. Synthesis of methyl trans-3-(3-methyl-4-(7-morpholinoquinoxalin-2-yl)-1H-pyrazol-1-yl)cyclobutane-1-carboxylate

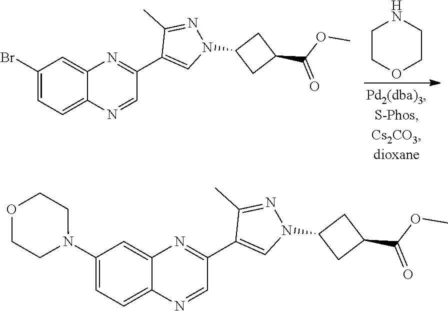

To a solution of methyl trans-3-(4-(7-bromoquinoxalin-2-yl)-3-methyl-1H-pyrazol-1-yl)cyclobutane-1-carboxylate (100.0 mg, 0.25 mmol) in dioxane (10 mL) were added morpholine (40.1 mg, 0.38 mmol), Pd₂(dba)₃ (11.7 mg, 0.0125 mmol), S-Phos (196.0 mg, 0.025 mmol) and Cs₂CO₃ (163.0 mg, 0.5 mmol) at rt. The mixture was stirred at 100° C. under inert atmosphere for 4 h. After cooling down to rt, the reaction mixture was poured into water (10 mL) and extracted with ethyl acetate (3×20 mL). The combined organic layers were washed with saturated brine (20 mL), dried over anhydrous sodium sulfate, filtered, and concentrated under reduced pressure. The resulting residue was purified by reverse-phase chromatography to provide the desired product (74.0 mg, 73% yield) as a pale white solid. MS (ESI) m/z=408.2 [M+H]⁺.

Step 4. Synthesis of (trans-3-(3-methyl-4-(7-morpholinoquinoxalin-2-yl)-1H-pyrazol-1-yl)cyclobutyl)methanol

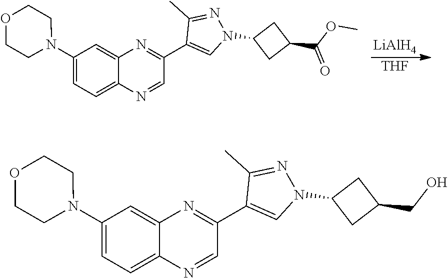

To a solution of methyl trans-3-(3-methyl-4-(7-morpholinoquinoxalin-2-yl)-1H-pyrazol-1-yl)cyclobutane-1-carboxylate (100.0 mg, 0.25 mmol) in THE (5 mL) was added LiAlH₄ (33.1 mg, 0.75 mmol) at 0° C. in portions. The reaction mixture was stirred at the same temperature for 1 h, before it was quenched with solid Na₂SO₄ and diluted with EtOAc (15 mL). The suspension was filtered through Celite and the filtrate was concentrated to provide the crude product (93.1 mg) as a pale white solid which was used in the next step directly. MS (ESI) m/z=380.2 [M+H]⁺.

Step 5. Synthesis of trans-3-(3-methyl-4-(7-morpholinoquinoxalin-2-yl)-1H-pyrazol-1-yl)cyclobutane-1-carbaldehyde

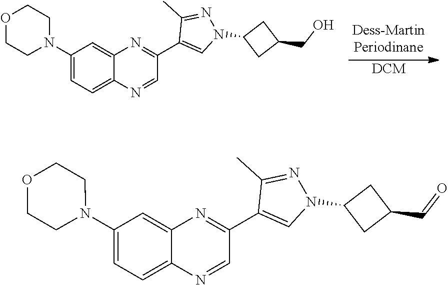

To a suspension of (trans-3-(3-methyl-4-(7-morpholinoquinoxalin-2-yl)-1H-pyrazol-1-yl)cyclobutyl)methanol (93.1 mg, 0.25 mmol) and NaHCO₃ (21.2 mg, 0.25 mmol) in DCM (5 mL) was added Dess-Martin Periodinane (318 mg, 0.75 mmol) at 0° C. in 2 portions. The reaction mixture was stirred at rt for 2 h, before it was diluted with DCM (10 mL) and filtered through Celite. The filtrate was concentrated to provide the crude product (92.9 mg) as a pale white solid which was used in the next step directly. MS (ESI) m/z=378.2 [M+H]⁺.

Step 6. Synthesis of (E)-3-(trans-3-(3-methyl-4-(7-morpholinoquinoxalin-2-yl)-1H-pyrazol-1-yl)cyclobutyl)acrylonitrile

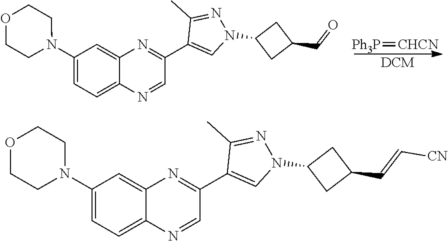

To a solution of trans-3-(4-(6-bromoquinoxalin-2-yl)-1H-pyrazol-1-yl)cyclobutane-1-carbaldehyde (93.1 mg, 0.25 mmol) in DCM (5 mL) was added triphenyl(cyanomethylene)phosphorene (83 mg, 0.28 mmol) in DCM (2 mL) dropwise at 0° C. Then the reaction mixture was stirred at rt for 30 min. The reaction was quenched with water (5 mL) and extracted with DCM (3×5 mL). The combined organic layers were dried over anhydrous Na₂SO₄, filtered, and concentrated under reduced pressure. The resulting residue was purified by reverse-phase chromatography to provide the desired product (68.0 mg, 68% yield for 3 steps) as a pale white solid. MS (ESI) m/z=401.2 [M+H]⁺.

Step 7. Synthesis of 3-(trans-3-(3-methyl-4-(7-morpholinoquinoxalin-2-yl)-1H-pyrazol-1-yl)cyclobutyl)propan-1-amine

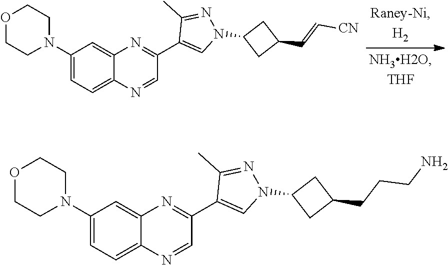 [SMILES: Cc1nn([C@H]2C[C@H](/C=C/C#N)C2)cc1-c1cnc2ccc(N3CCOCC3)cc2n1 ; Cc1nn([C@H]2C[C@H](CCCN)C2)cc1-c1cnc2ccc(N3CCOCC3)cc2n1]

A mixture of (E)-3-(trans-3-(3-methyl-4-(7-morpholinoquinoxalin-2-yl)-1H-pyrazol-1-yl)cyclobutyl)acrylonitrile (68.0 mg, 0.17 mmol) and Raney-Ni catalyst (13.6 mg, 20% wt) in THF (5 mL) and NH₃·H₂O (0.5 mL) was stirred at rt under hydrogen balloon for 20 min. Then the reaction mixture was filtered through Celite and the cake was washed with EtOAc (10 mL). The filtrate was concentrated and purified by reverse-phase chromatography to provide the desired product (32.0 mg, 46% yield) as a yellow solid. MS (ESI) m/z=407.3 [M+H]⁺.

Step 8. Synthesis of 3-(5-((3-(trans-3-(3-methyl-4-(7-morpholinoquinoxalin-2-yl)-1H-pyrazol-1-yl)cyclobutyl)propyl)amino)-1,3-dioxo-2,3-dihydro-TH-inden-2-yl)piperidine-2,6-dione

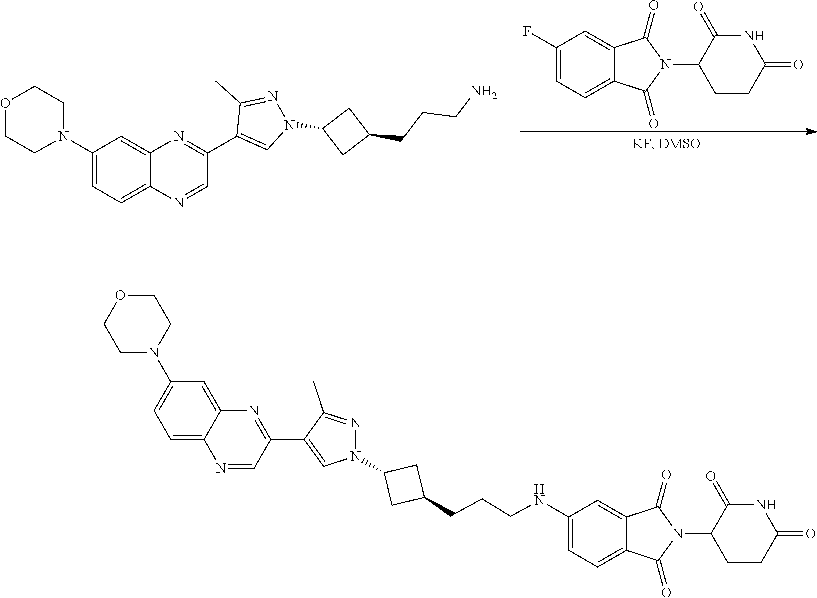 [SMILES: Cc1nn([C@H]2C[C@H](CCCN)C2)cc1-c1cnc2ccc(N3CCOCC3)cc2n1 ; O=C1CCC(N2C(=O)c3ccc(F)cc3C2=O)C(=O)N1 ; Cc1nn([C@H]2C[C@H](CCCNc3ccc4c(c3)C(=O)N(C3CCC(=O)NC3=O)C4=O)C2)cc1-c1cnc2ccc(N3CCOCC3)cc2n1]

To a solution of 3-(trans-3-(3-methyl-4-(7-morpholinoquinoxalin-2-yl)-1H-pyrazol-1-yl)cyclobutyl)propan-1-amine (15 mg, 0.037 mmol) and 2-(2,6-dioxopiperidin-3-yl)-5-fluoroisoindoline-1,3-dione (15.2 mg, 0.055 mmol) in DMSO (0.5 mL) was added KF (6.4 mg, 0.11 mmol). The reaction mixture was stirred at 130° C. for 25 min. After cooling down to rt, the solution was poured into water (5 mL) and extracted with ethyl acetate (3×5 mL). The combined organic layers were washed with saturated brine (5 mL), dried over anhydrous sodium sulfate, filtered, and concentrated under reduced pressure. The resulting residue was purified by reverse-phase chromatography to provide the desired product (2.7 mg, 11% yield) as a yellow solid. MS (ESI) m/z=663.4 [M+H]⁺.

### Example 358. 2-(2,6-Dioxopiperidin-3-yl)-5-((3-(trans-3-(4-(quinoxalin-2-yl)-3-(trifluoromethyl)-1H-pyrazol-1-yl)cyclobutyl)propyl)amino)isoindoline-1,3-dione (GS-400)

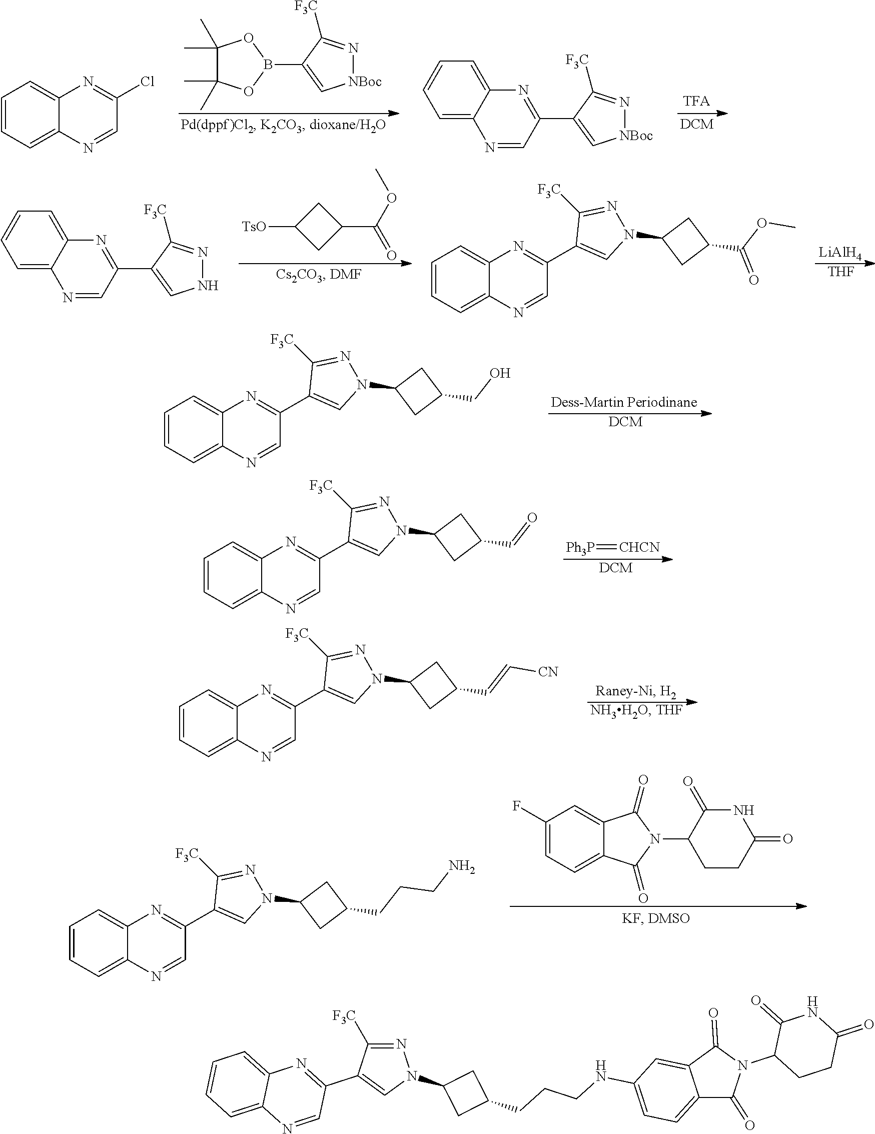

GS-400 was synthesized following the standard procedures for preparing GS-399 (3.0 mg, 12% yield) as a light-yellow solid. MS (ESI) m/z=632.4 [M+H]⁺.

### Example 359. 2-(2,6-Dioxopiperidin-3-yl)-5-((3-(trans-3-(4-(6-(piperazin-1-yl)quinoxalin-2-yl)-1H-pyrazol-1-yl)cyclobutyl)propyl)amino)isoindoline-1,3-dione (GS-401)

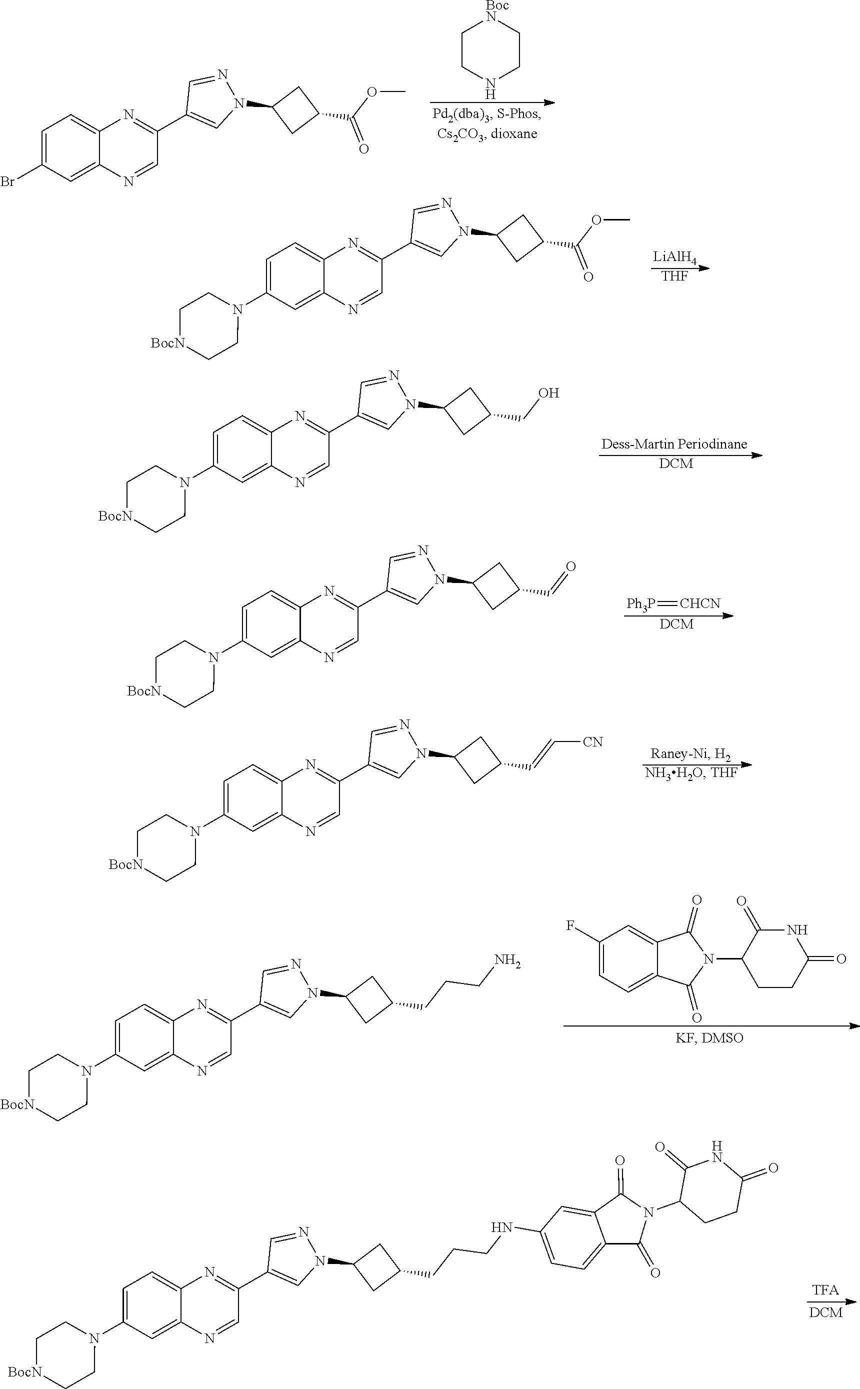

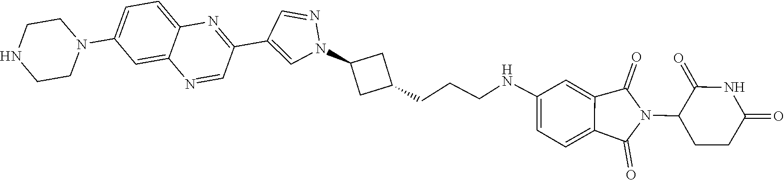 [SMILES: O=C1CCC(N2C(=O)c3ccc(NCCC[C@H]4C[C@H](n5cc(-c6cnc7cc(N8CCNCC8)ccc7n6)cn5)C4)cc3C2=O)C(=O)N1]

GS-401 was synthesized following the standard procedures for preparing GS-399 (6.7 mg, 39% yield) as a light-yellow solid. MS (ESI) m/z=648.4 [M+H]⁺.

### Example 360. 2-(2,6-Dioxopiperidin-3-yl)-5-((3-(cis-3-(4-(6-morpholinoquinoxalin-2-yl)-1H-pyrazol-1-yl)cyclobutyl)propyl)amino)isoindoline-1,3-dione (GS-402)

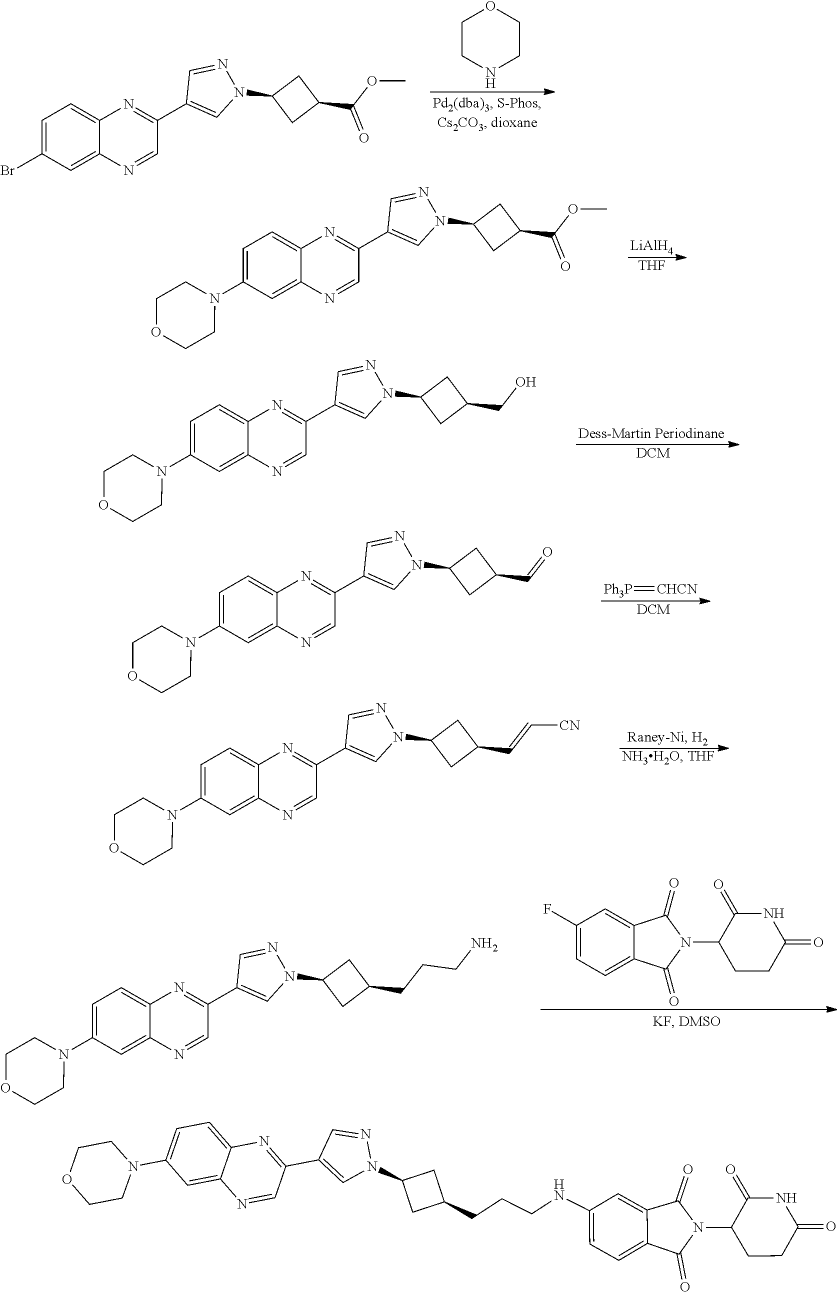

GS-402 was synthesized following the standard procedures for preparing GS-399 (0.7 mg, 9% yield over 6 steps) as a light-yellow solid. MS (ESI) m/z=649.4 [M+H]⁺.

### Example 361. 2-(2,6-Dioxopiperidin-3-yl)-5-((3-(trans-3-(4-(5-morpholinoquinoxalin-2-yl)-1H-pyrazol-1-yl)cyclobutyl)propyl)amino)isoindoline-1,3-dione (GS-403)

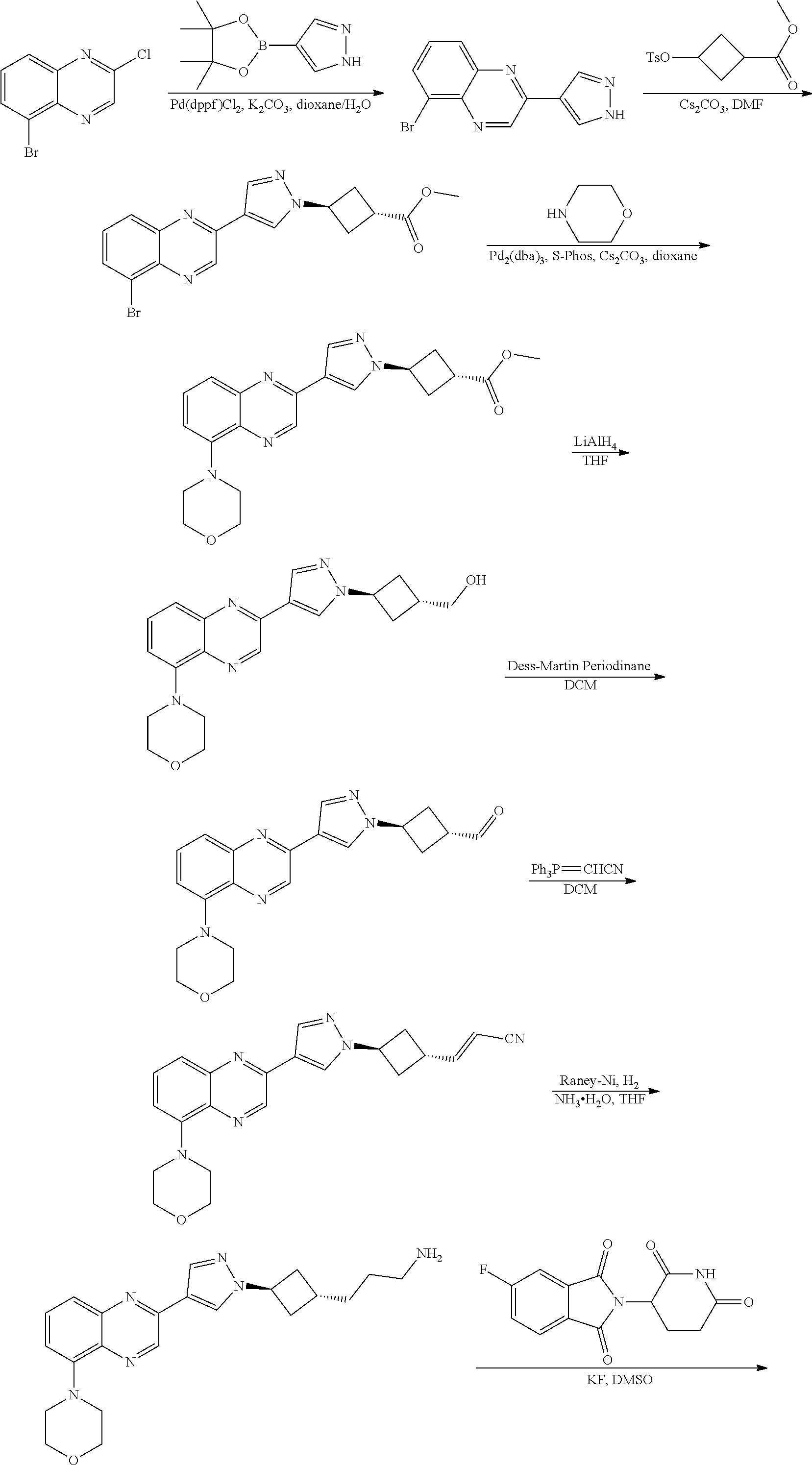

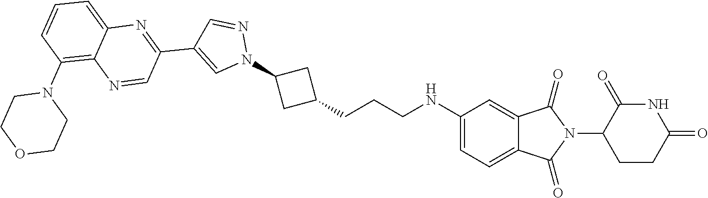 [SMILES: O=C1CCC(N2C(=O)c3ccc(NCCC[C@H]4C[C@H](n5cc(-c6cnc7c(N8CCOCC8)cccc7n6)cn5)C4)cc3C2=O)C(=O)N1]

GS-403 was synthesized following the standard procedures for preparing GS-399 (3.2 mg, 22% yield) as a light-yellow solid. MS (ESI) m/z=649.5 [M+H]⁺.

### Example 362. 2-(2,6-Dioxopiperidin-3-yl)-5-((3-(trans-3-(4-(5-(4-methylpiperazin-1-yl)quinoxalin-2-yl)-1H-pyrazol-1-yl)cyclobutyl)propyl)amino)isoindoline-1,3-dione (GS-404)

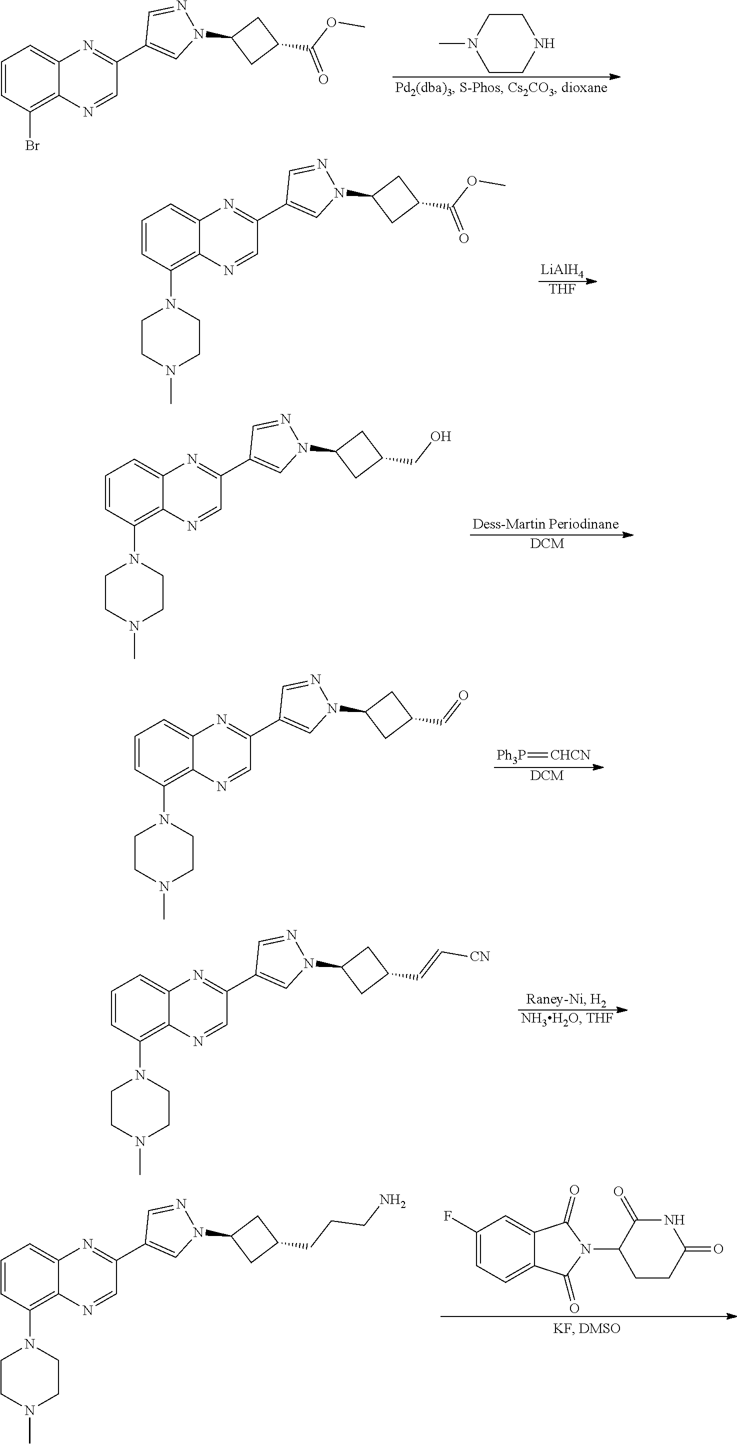

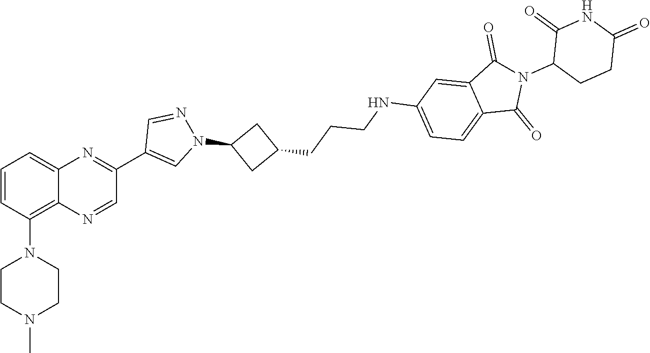 [SMILES: CN1CCN(c2cccc3nc(-c4cnn([C@H]5C[C@H](CCCNc6ccc7c(c6)C(=O)N(C6CCC(=O)NC6=O)C7=O)C5)c4)cnc23)CC1]

GS-404 was synthesized following the standard procedures for preparing GS-399 (7.5 mg, 31% yield) as a light-yellow solid. MS (ESI) m/z=662.5 [M+H]⁺.

### Example 363. 2-(2,6-Dioxopiperidin-3-yl)-5-((3-(trans-3-(4-(5-(piperazin-1-yl)quinoxalin-2-yl)-1H-pyrazol-1-yl)cyclobutyl)propyl)amino)isoindoline-1,3-dione (GS-405)

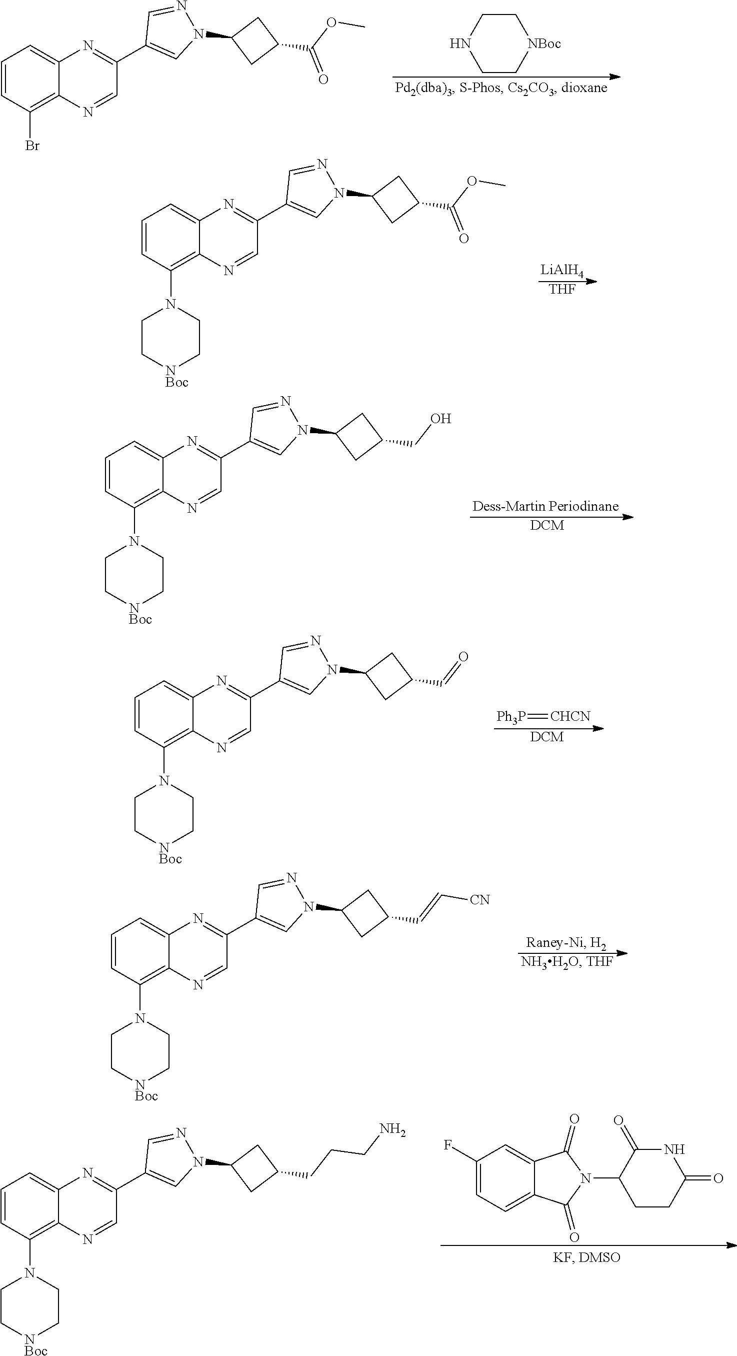

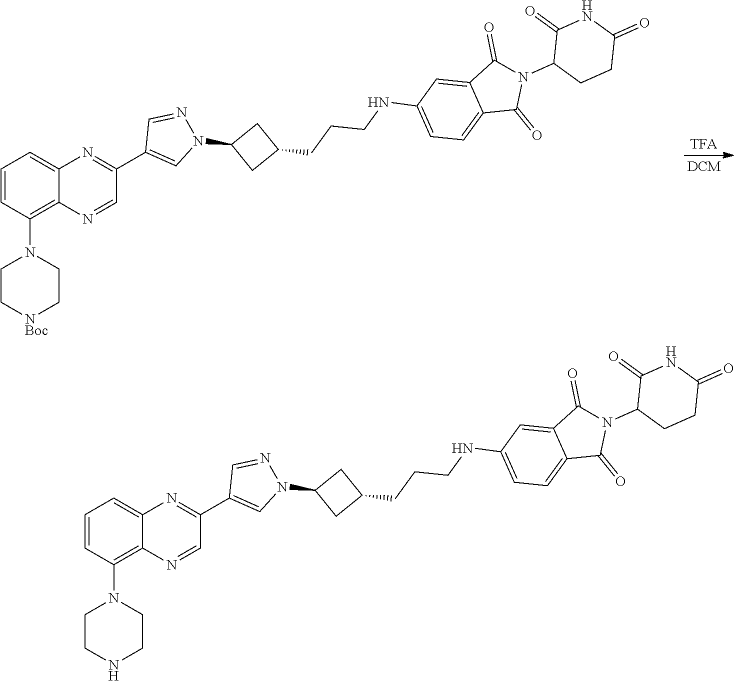 [SMILES: CC(C)(C)OC(=O)N1CCN(c2cccc3nc(-c4cnn([C@H]5C[C@H](CCCNc6ccc7c(c6)C(=O)N(C6CCC(=O)NC6=O)C7=O)C5)c4)cnc23)CC1 ; O=C1CCC(N2C(=O)c3ccc(NCCC[C@H]4C[C@H](n5cc(-c6cnc7c(N8CCNCC8)cccc7n6)cn5)C4)cc3C2=O)C(=O)N1]

GS-405 was synthesized following the standard procedures for preparing GS-399 (6.2 mg, 35% yield) as a light-yellow solid. MS (ESI) m/z=648.4 [M+H]⁺.

### Example 364. 5-((3-(cis-3-(4-(7-(8-Oxa-3-azabicyclo[3.2.1]octan-3-yl)quinoxalin-2-yl)-1H-pyrazol-1-yl)cyclobutyl)propyl)amino)-2-(2,6-dioxopiperidin-3-yl)isoindoline-1,3-dione (GS-406)

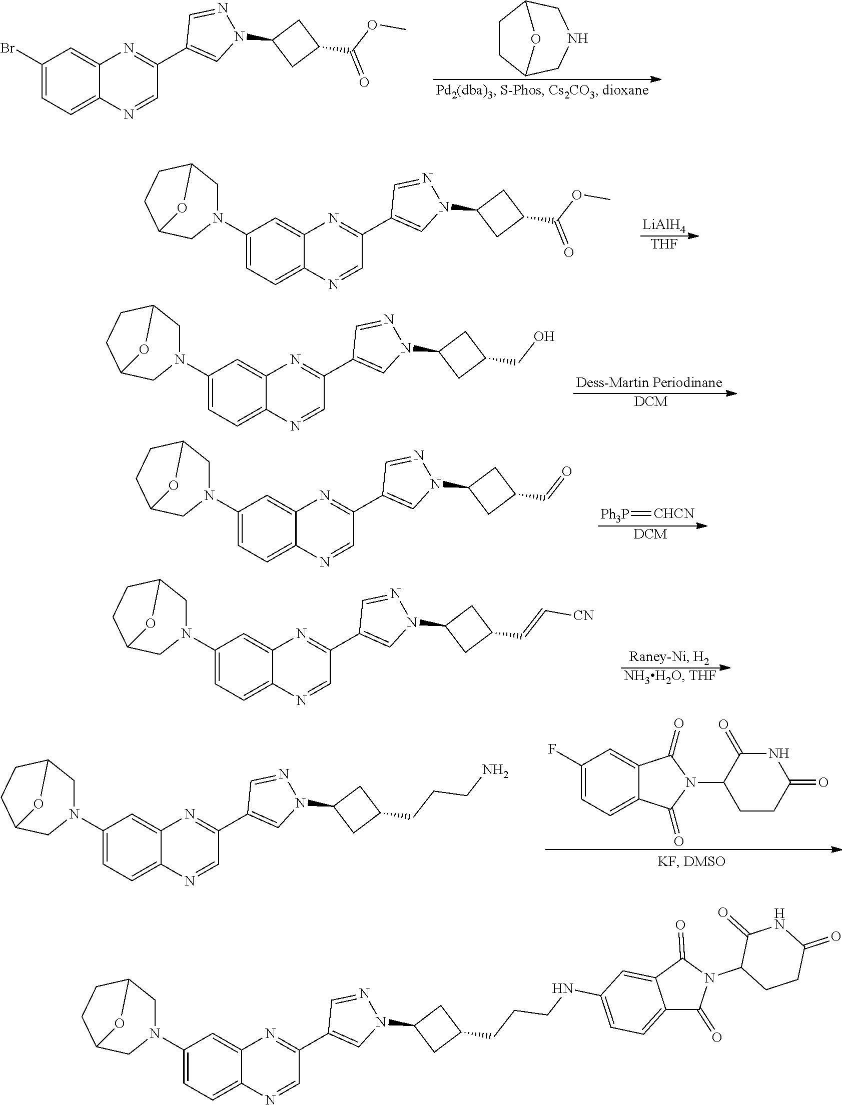

GS-406 was synthesized following the standard procedures for preparing GS-399 (15.8 mg, 14% yield over 6 steps) as a light-yellow solid. MS (ESI) m/z=675.4 [M+H]⁺.

### Example 365. 2-(2,6-Dioxopiperidin-3-yl)-5-((3-(trans-3-(3-methyl-4-(7-((1R,4R)-5-methyl-2,5-diazabicyclo[2.2.1]heptan-2-yl)quinoxalin-2-yl)-1H-pyrazol-1-yl)cyclobutyl)propyl)amino)isoindoline-1,3-dione (GS-407)

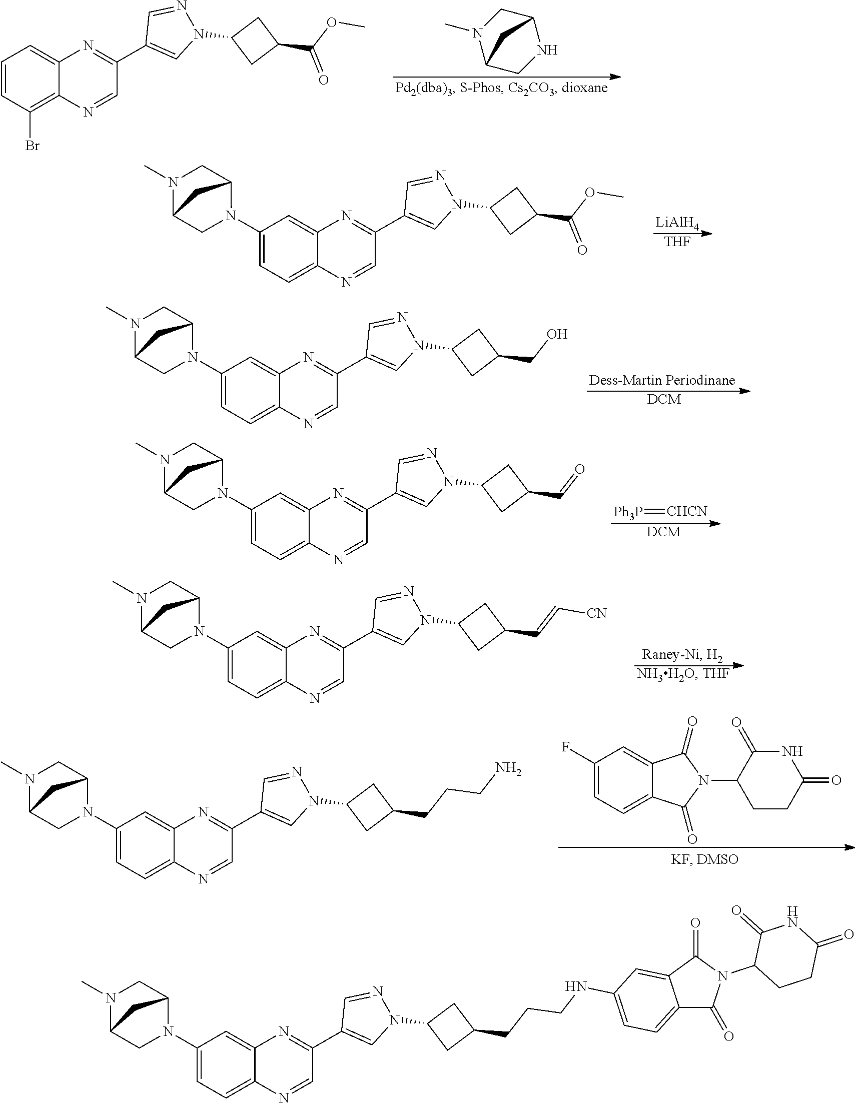

GS-407 was synthesized following the standard procedures for preparing GS-399 (1.6 mg, 100% yield) as a light-yellow solid. MS (ESI) m/z=688.5 [M+H]⁺.

### Example 366. 2-(2,6-Dioxopiperidin-3-yl)-5-((3-(trans-3-(4-(7-((3S,5R)-3,4,5-trimethylpiperazin-1-yl)quinoxalin-2-yl)-1H-pyrazol-1-yl)cyclobutyl)propyl)amino)isoindoline-1,3-dione (GS-408)

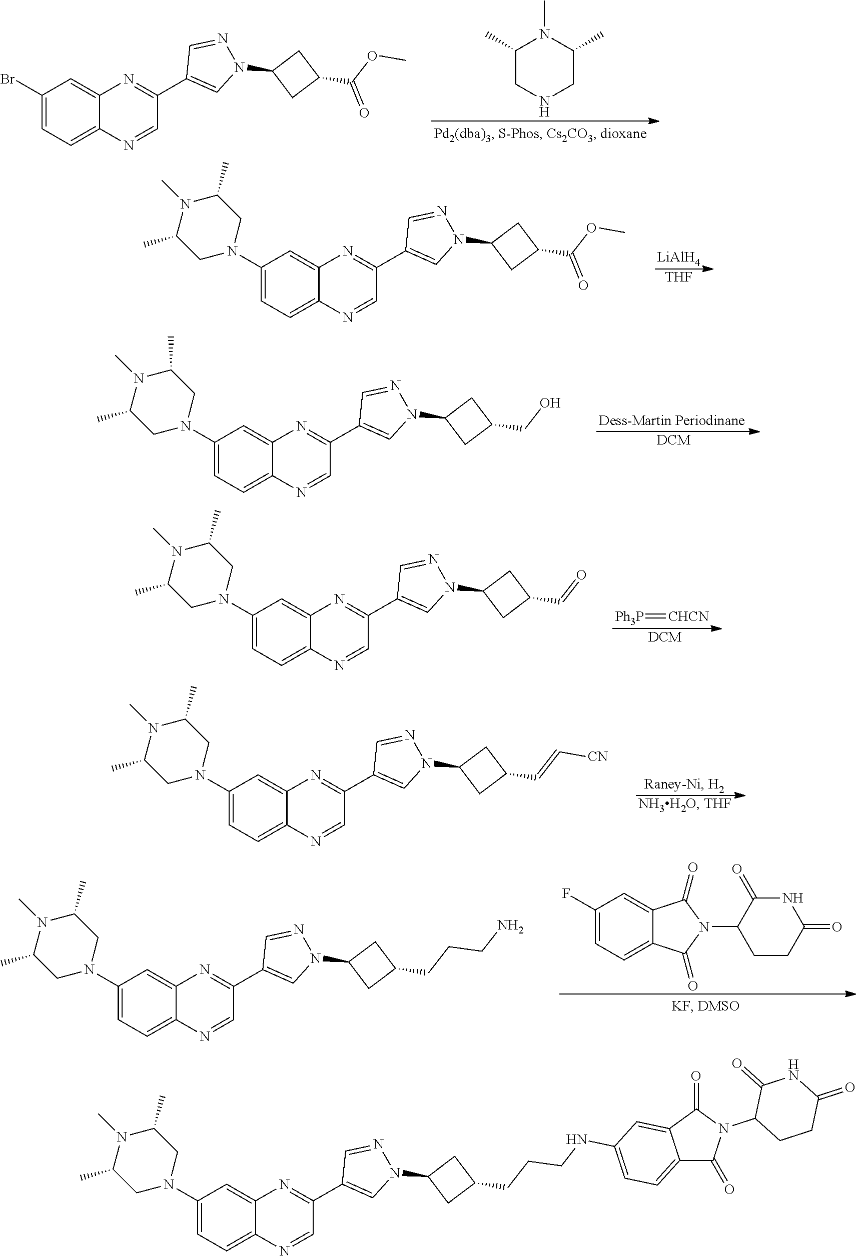

GS-408 was synthesized following the standard procedures for preparing GS-399 (0.76 mg, 8% yield over 6 steps) as a yellow solid. MS (ESI) m/z=690.4 [M+H]⁺.

### Example 367. 2-(2,6-Dioxopiperidin-3-yl)-5-((3-(trans-3-(4-(7-(8-methyl-3,8-diazabicyclo[3.2.1]octan-3-yl)quinoxalin-2-yl)-1H-pyrazol-1-yl)cyclobutyl)propyl)amino)isoindoline-1,3-dione (GS-409)

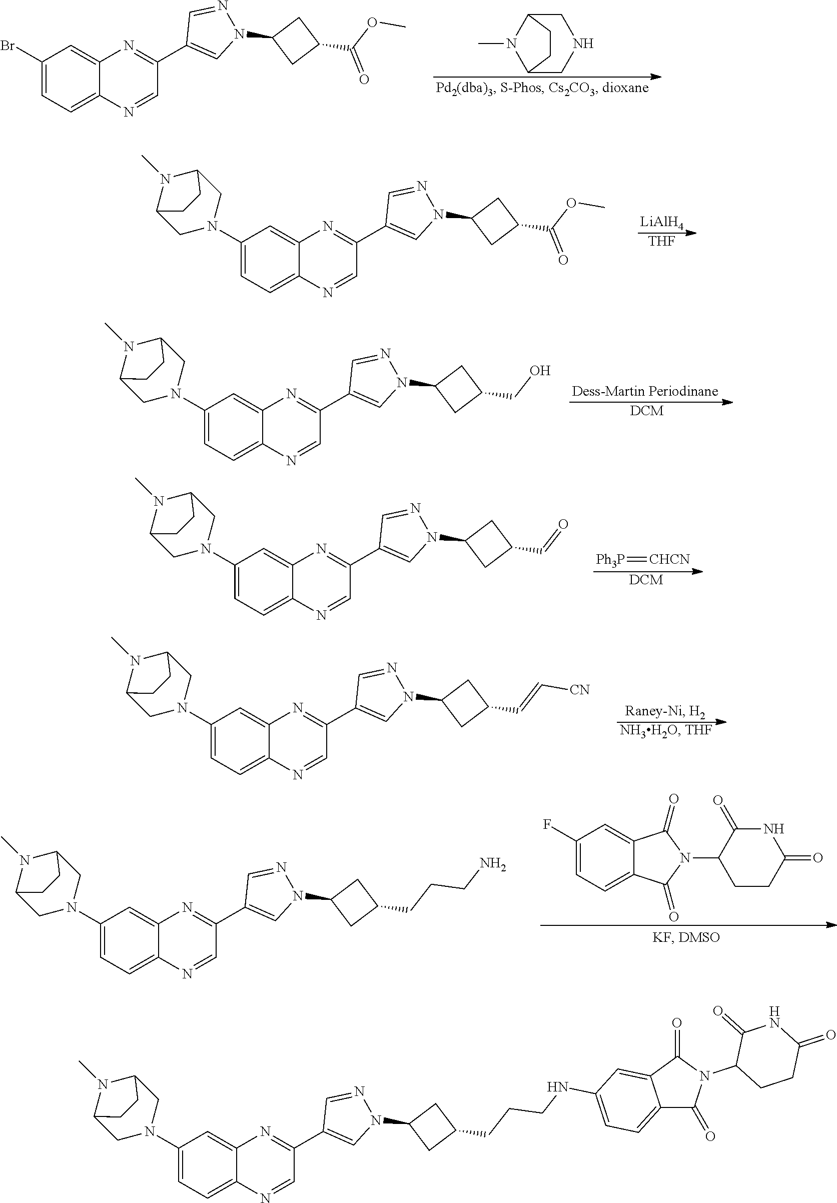

GS-409 was synthesized following the standard procedures for preparing GS-399 (7.5 mg, 27° % yield) as a light-yellow solid. MS (ESI) m/z=688.4 [M+H]⁺.

### Example 368. 5-((3-(trans-3-(4-(7-((1R,4R)-2-Oxa-5-azabicyclo[2.2.1]heptan-5-yl)quinoxalin-2-yl)-1H-pyrazol-1-yl)cyclobutyl)propyl)amino)-2-(2,6-dioxopiperidin-3-yl)isoindoline-1,3-dione (GS-410)

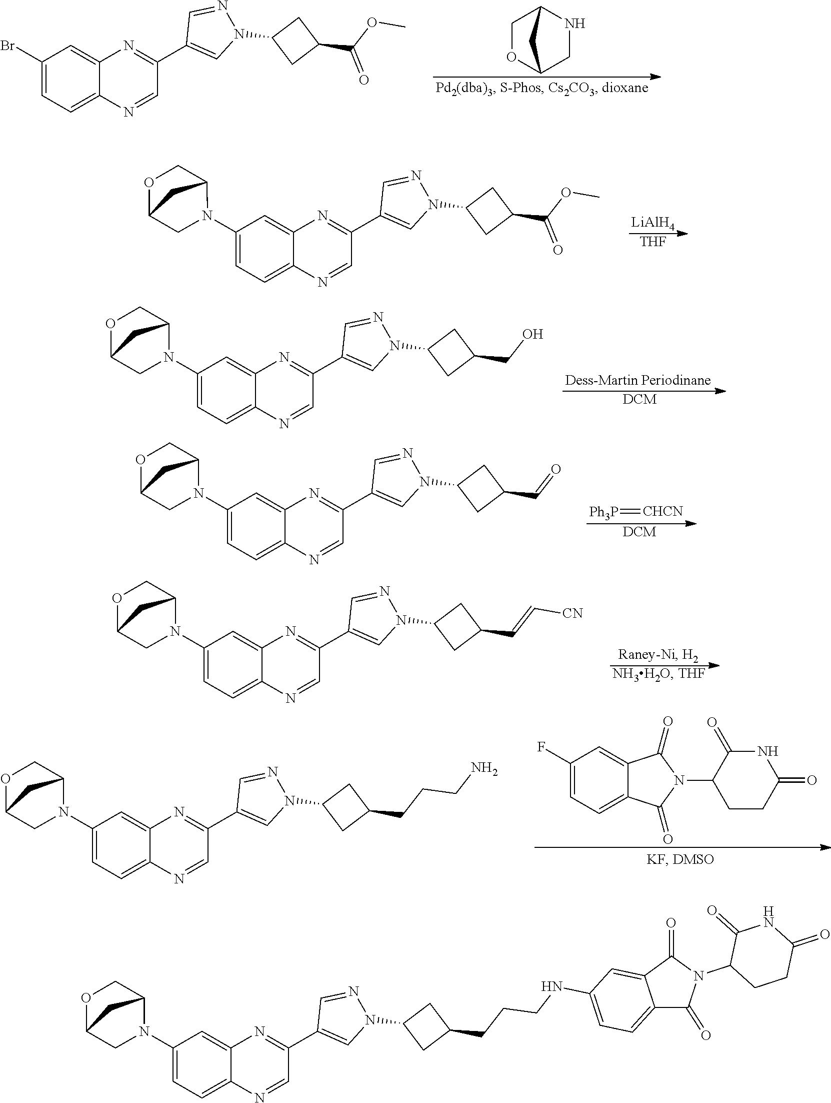

GS-410 was synthesized following the standard procedures for preparing GS-399 (6 mg, 7% yield over 6 steps) as a light-yellow solid. MS (ESI) m/z=661.4 [M+H]⁺.

### Example 369. 2-(2,6-Dioxopiperidin-3-yl)-5-((3-(cis-3-(4-(7-((1R,4R)-5-methyl-2,5-diazabicyclo[2.2.1]heptan-2-yl)quinoxalin-2-yl)-1H-pyrazol-1-yl)cyclobutyl)propyl)amino)isoindoline-1,3-dione (GS-411)

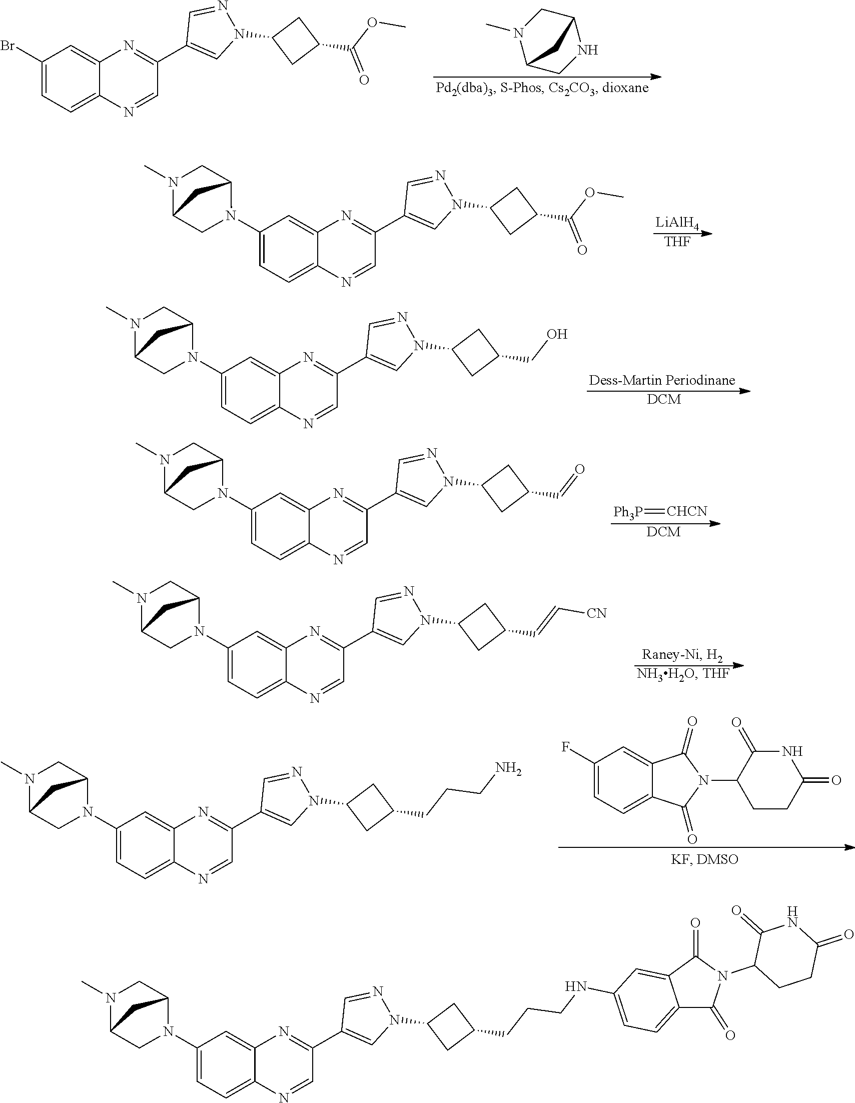

GS-411 was following the standard procedures for preparing GS-399 (24.8 mg, 10% yield over 6 steps) as a yellow solid. MS (ESI) m/z=674.4 [M+H]⁺.

### Example 370. 2-(2,6-Dioxopiperidin-3-yl)-5-((3-(cis-3-(4-(6-(4-methylpiperazin-1-yl)quinoxalin-2-yl)-1H-pyrazol-1-yl)cyclobutyl)propyl)amino)isoindoline-1,3-dione (GS-412)

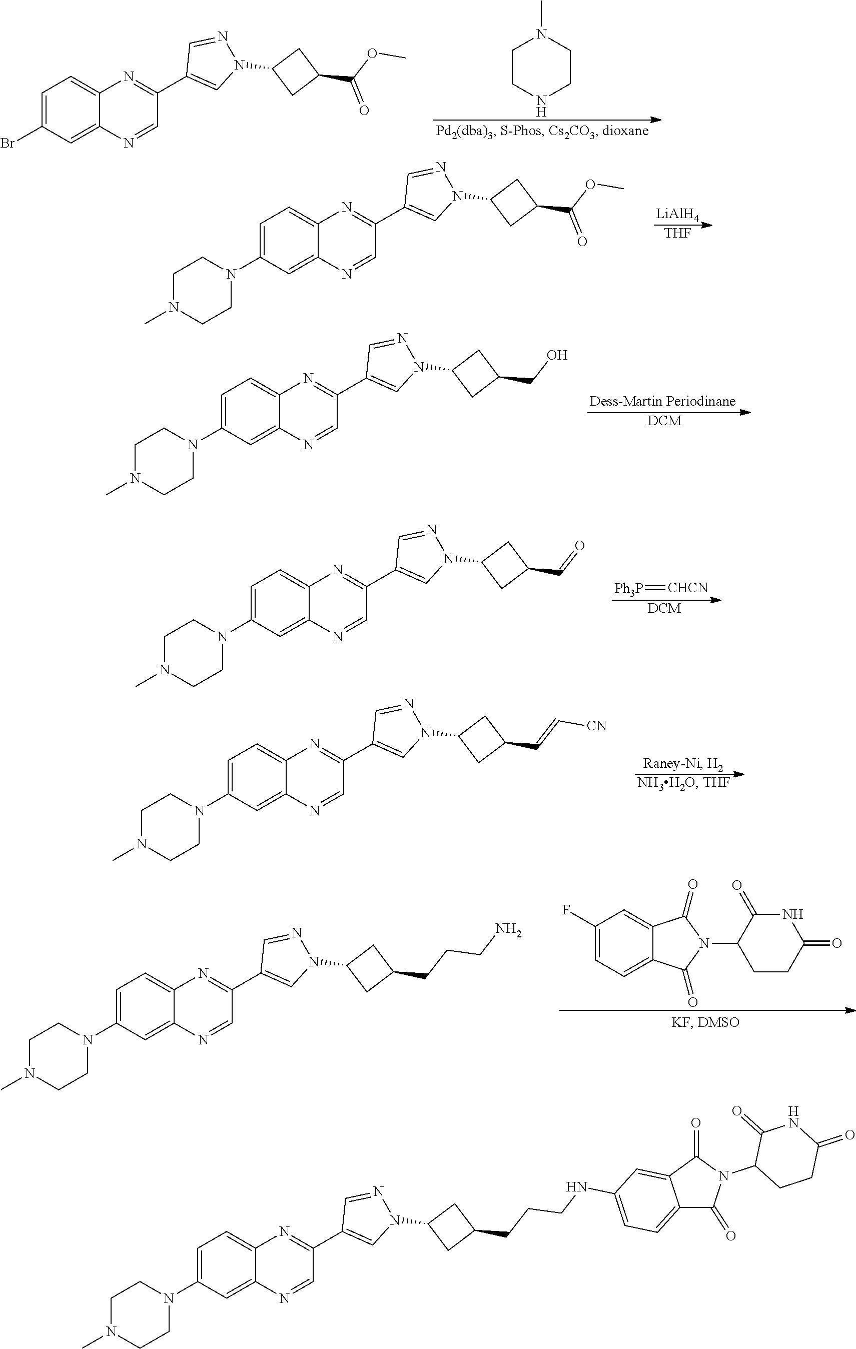

GS-412 was synthesized following the standard procedure for preparing GS-399 (6.8 mg, 36% yield) as a light-yellow solid. MS (ESI) m/z=662.4 [M+H]⁺.

### Example 371. 2-(2,6-Dioxopiperidin-3-yl)-5-((3-(trans-3-(4-(6-(4-methylpiperazin-1-yl)pyridin-2-yl)-1H-pyrazol-1-yl)cyclobutyl)propyl)amino)isoindoline-1,3-dione (GS-413)

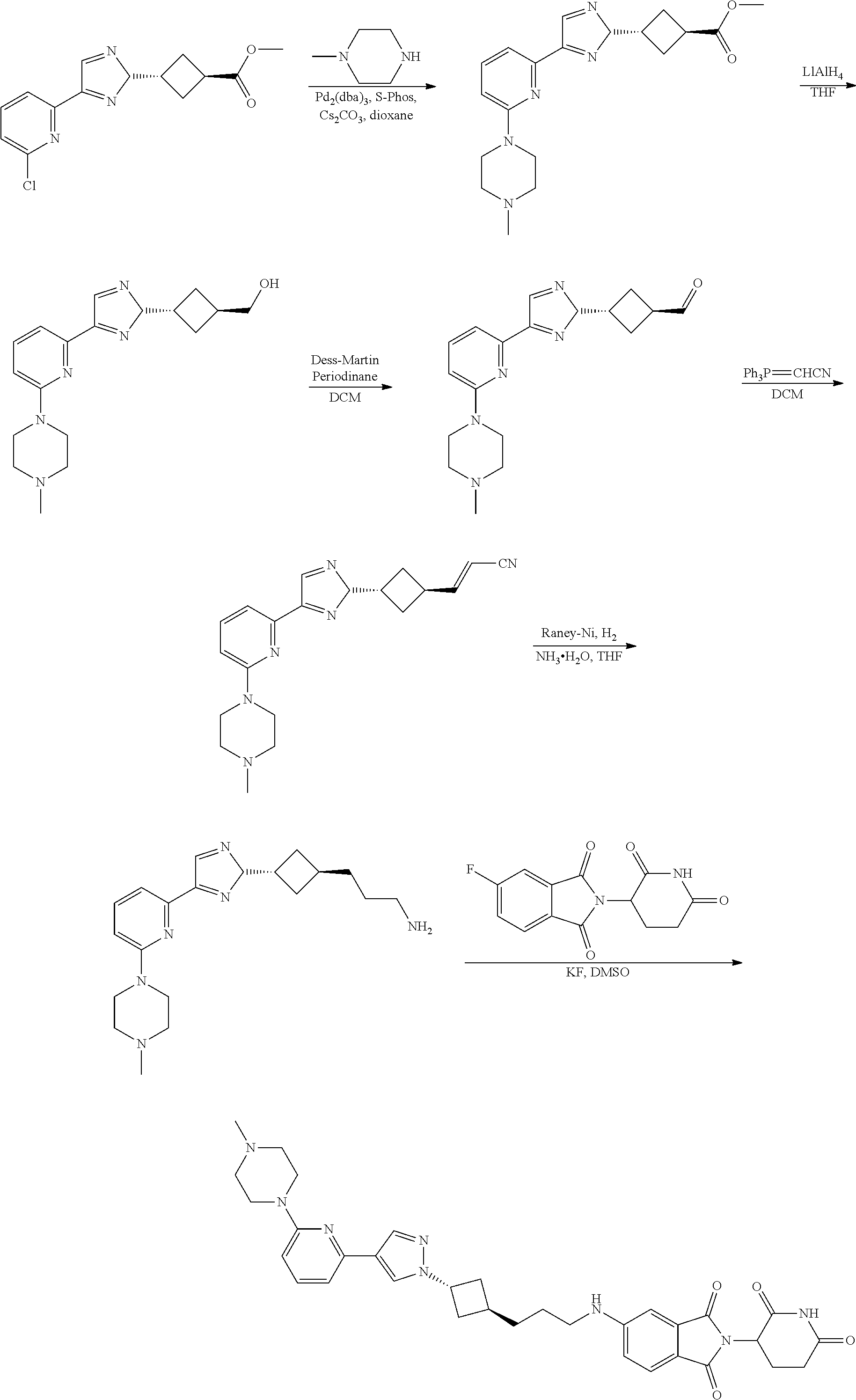

GS-413 was synthesized following the standard procedures for preparing GS-399 (2.2 mg, 11% yield) as a light-yellow solid. MS (ESI) m/z=611.3 [M+H]⁺.

### Example 372. 2-(2,6-Dioxopiperidin-3-yl)-5-((3-(trans-3-(4-(7-(4-methyl-4,7-diazaspiro[2.5]octan-7-yl)quinoxalin-2-yl)-1H-pyrazol-1-yl)cyclobutyl)propyl)amino)isoindoline-1,3-dione (GS-414)

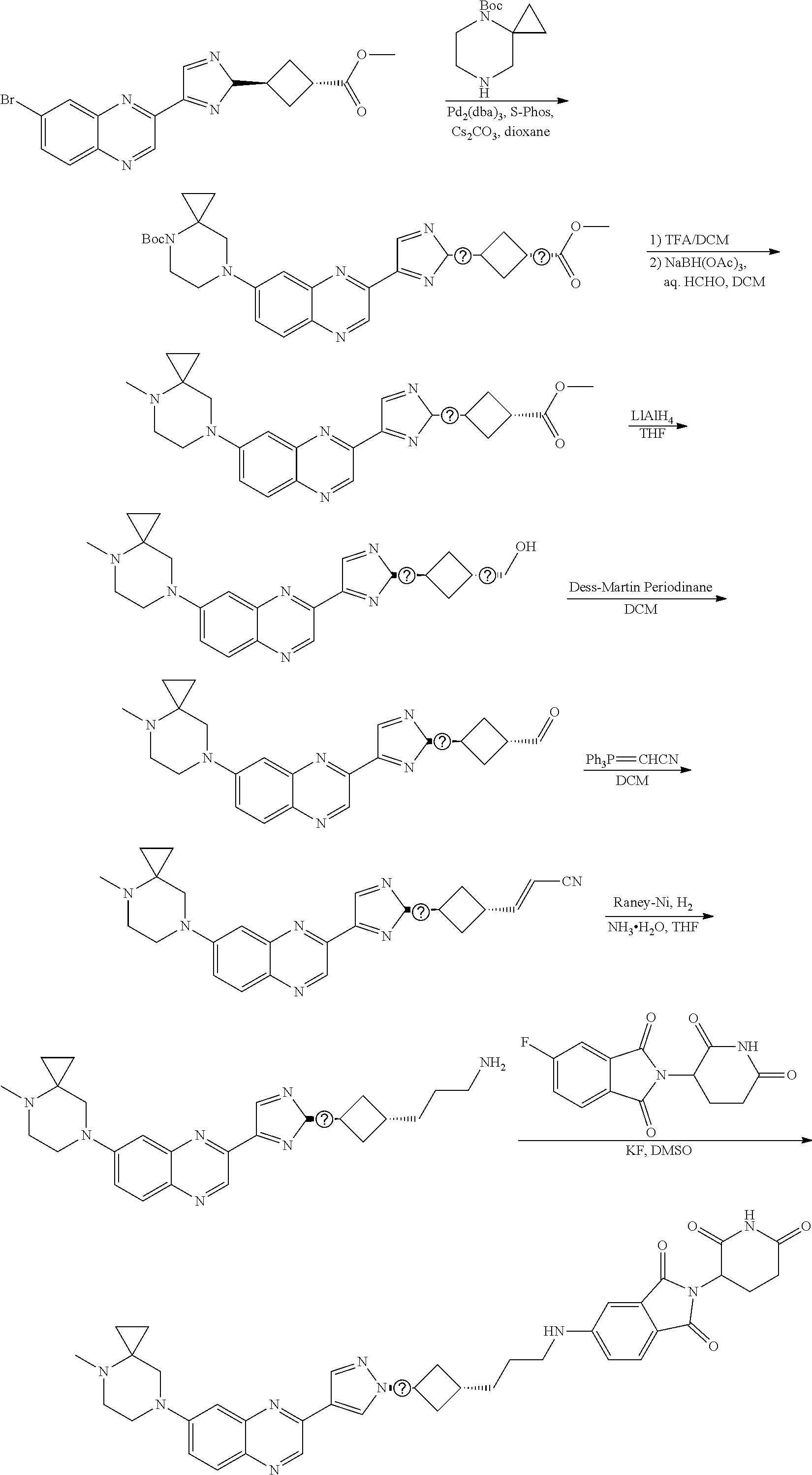

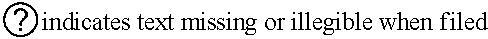

Step 1. Synthesis of tert-butyl 7-(3-(1-(trans-3-(methoxycarbonyl)cyclobutyl)-1H-pyrazol-4-yl) quinoxalin-6-yl)-4,7-diazaspiro[2.5]octane-4-carboxylate

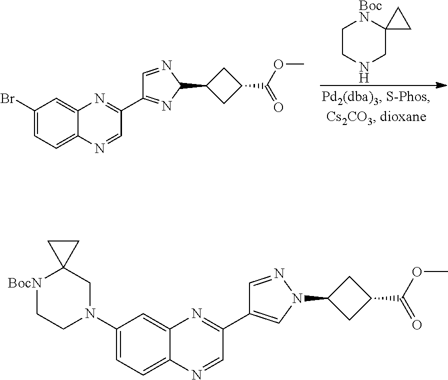 [SMILES: COC(=O)[C@H]1C[C@H](C2N=CC(c3cnc4ccc(Br)cc4n3)=N2)C1 ; CC(C)(C)OC(=O)N1CCNCC12CC2 ; COC(=O)[C@H]1C[C@H](n2cc(-c3cnc4ccc(N5CCN(C(=O)OC(C)(C)C)C6(CC6)C5)cc4n3)cn2)C1]

To a suspension of methyl trans-3-(4-(7-bromoquinoxalin-2-yl)-1H-pyrazol-1-yl)cyclobutane-1-carboxylate (50.0 mg, 0.13 mmol) in dioxane (3 mL) were added tert-butyl 4,7-diazaspiro[2.5]octane-4-carboxylate (55.2 mg, 0.26 mmol), Cs₂CO₃ (84.8 mg, 0.26 mmol), S-Phos (12.3 mg, 0.03 mmol) and Pd₂(dba)₃ (9.5 mg, 0.01 mmol) at rit. The reaction mixture was purged with N₂ and stirred at 100° C. for 12 h. After cooling down to rt, the reaction was diluted with EtOAc (10 mL) and filtered. The filtrate was concentrated and purified by reverse-phase chromatography to provide the desired product (45.0 mg, 67% yield) as a yellow solid. MS (ESI) m/z=519.4 [M+H]⁺.

Step 2. methyl trans-3-(4-(7-(4-methyl-4,7-diazaspiro[2.5]octan-7-yl)quinoxalin-2-yl)-1H-pyrazol-1-yl)cyclobutane-1-carboxylate

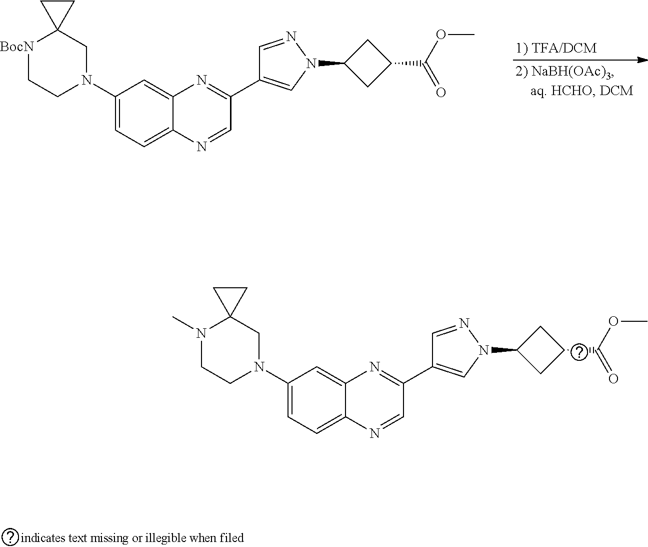

A solution of tert-butyl 7-(3-(1-(trans-3-(methoxycarbonyl)cyclobutyl)-1H-pyrazol-4-yl)quin oxalin-6-yl)-4,7-diazaspiro[2.5]octane-4-carboxylate (45.0 mg, 0.09 mmol) in DCM (2 mL) and TFA (1 mL) was stirred at rt for 1 h. Then the solution was concentrated under reduced pressure. The resulting residue was dissolved in DCM (3 mL), then aq. HCHO solution (37 wt %, 0.01 mL) was added at 0° C. After stirring at 0° C. for 0.5 h, NaBH(OAc)₃ (52.8 mg, 0.25 mmol) was added and the solution was stirred at rt for another 2 h. The reaction mixture was concentrated and purified by silica gel column chromatography to provide the desired product (15.0 mg, 38% yield) as a yellow solid. MS (ESI) m/z=433.5 [M+H]⁺.

The remaining steps were performed according to the standard procedures for preparing GS-399 to provide the desired final product (0.5 mg, 2% yield over 5 steps) as a yellow solid. MS (ESI) m/z=688.5 [M+H]⁺.

### Example 373. 5-((3-(trans-3-(4-(7-((1R,4R)-2,5-Diazabicyclo[2.2.1]heptan-2-yl)quinoxalin-2-yl)-1H-pyrazol-1-yl)cyclobutyl)propyl)amino)-2-(2,6-dioxopiperidin-3-yl)isoindoline-1,3-dione (GS-415)

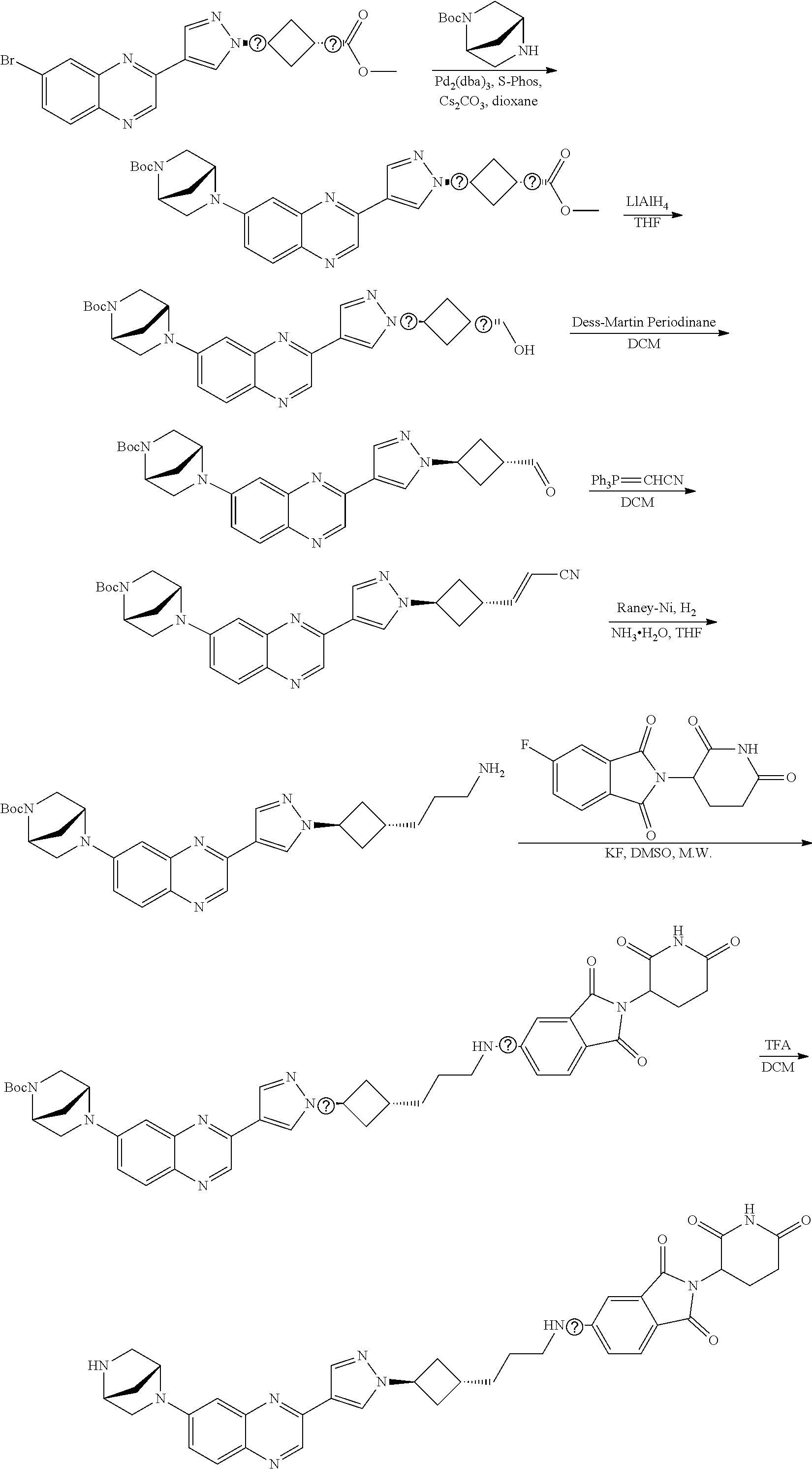

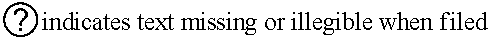

GS-415 was synthesized following the standard procedures for preparing GS-399 (2.9 mg, 3% yield over 7 steps) as a yellow solid. MS (ESI) m/z=660.5 [M+H]⁺.

### Example 374. 3-(5-((3-(trans-3-(4-(6-(4-Methylpiperazin-1-yl)pyridin-2-yl)-1H-pyrazol-1-yl)cyclobutyl)propyl)amino)-1,3-dioxo-2,3-dihydro-TH-inden-2-yl)piperdine-2,6-dione-(GS-416)

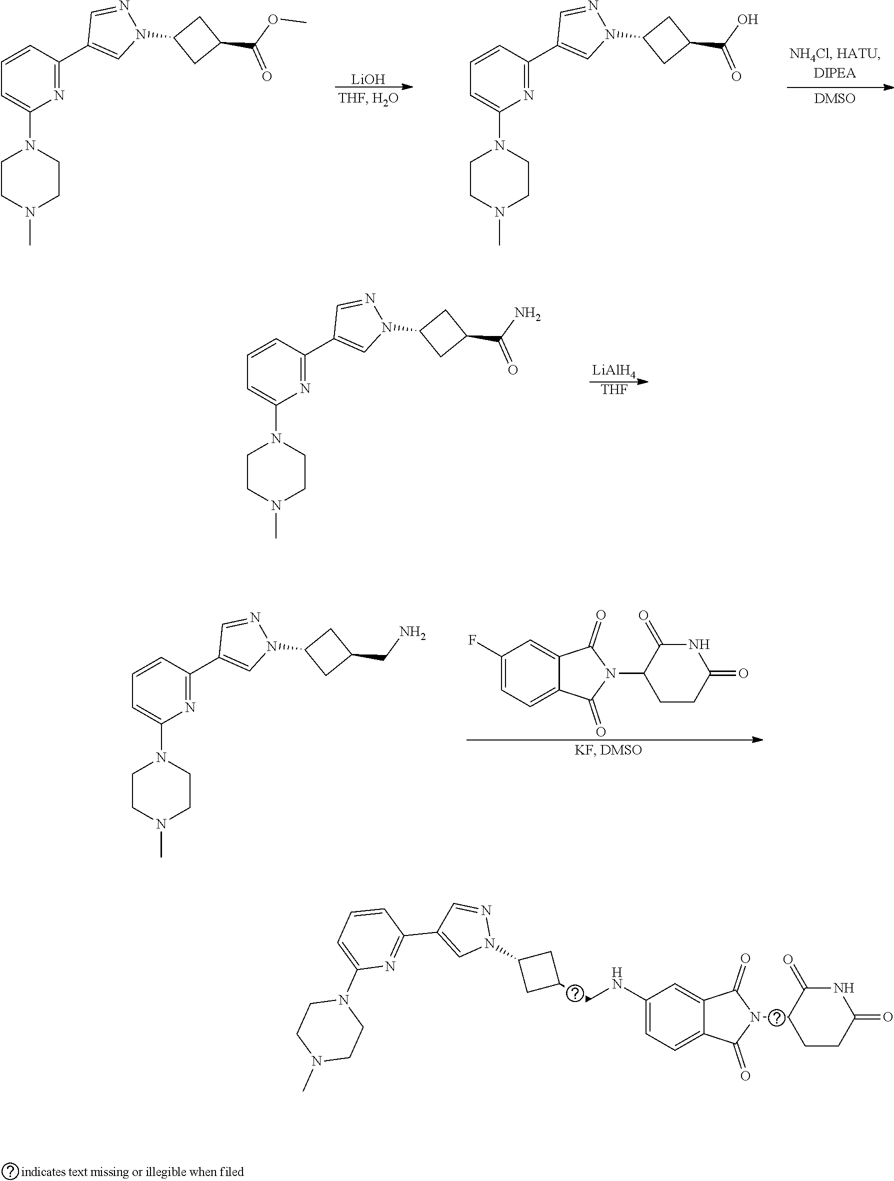

Step 1. Synthesis of trans-3-(4-(6-(4-methylpiperazin-1-yl)pyridin-2-yl)-1H-pyrazol-1-yl)cyclobutane-1-carboxylic acid

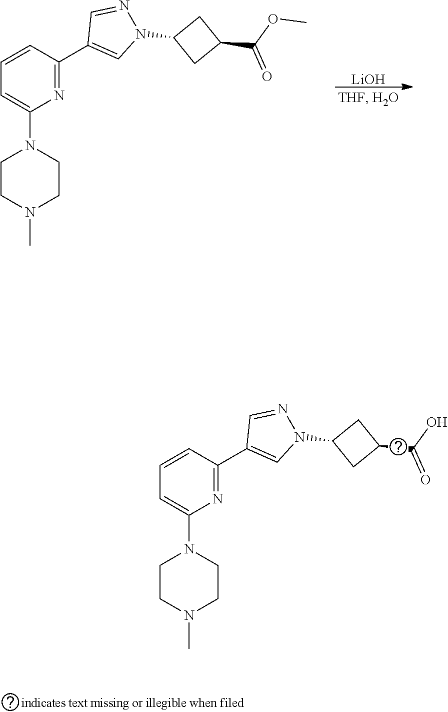 [SMILES: COC(=O)[C@H]1C[C@H](n2cc(-c3cccc(N4CCN(C)CC4)n3)cn2)C1 ; "LiOH" ; CN1CCN(c2cccc(-c3cnn([C@H]4C[C@H](C(=O)O)C4)c3)n2)CC1]

To a solution of methyl trans-3-(4-(6-(4-methylpiperazin-1-yl)pyridin-2-yl)-1H-pyrazol-1-yl)cyclobutane-1-carboxylate (100 mg, 0.28 mmol) in THF (10 mL) were added LiOH (24.3 mg, 0.58 mmol) and H₂O (2.0 mL). The solution was stirred at rt for 1 h. The reaction mixture was poured into aq. HCl (1.0 M, 3 mL) and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (70.1 mg, 73% yield) as a pale white solid. MS (ESI) m/z=342.2 [M+H]⁺.

Step 2. Synthesis of trans-3-(4-(6-(4-methylpiperazin-1-yl)pyridin-2-yl)-1H-pyrazol-1-yl)cyclobutane-1-carboxamide

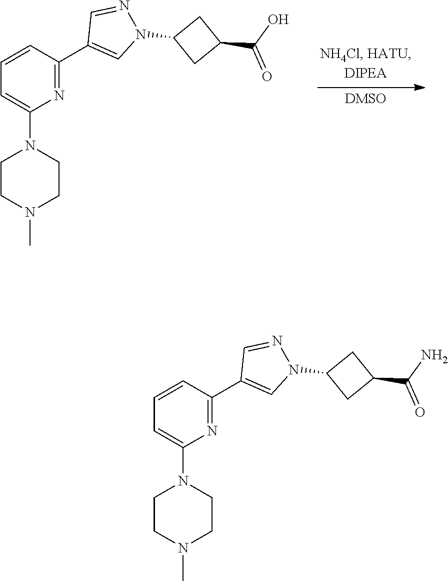 [SMILES: CN1CCN(c2cccc(-c3cnn([C@H]4C[C@H](C(=O)O)C4)c3)n2)CC1 ; CN1CCN(c2cccc(-c3cnn([C@H]4C[C@H](C(N)=O)C4)c3)n2)CC1]

A mixture of trans-3-(4-(6-(4-methylpiperazin-1-yl)pyridin-2-yl)-1H-pyrazol-1-yl)cyclobutane-1-carboxylic acid (70.1 mg, 0.21 mmol), NH₄Cl (45.2 mg, 0.84 mmol), HATU (118.1 mg, 0.31 mmol) and DIPEA (135 mg, 1.1 mmol) in DMSO (2.0 mL) was stirred at rt for 16 h. The reaction mixture was poured into water (15 mL) and extracted with ethyl acetate (3×10 mL). The combined organic layers were washed with saturated brine (10 mL), dried over anhydrous sodium sulfate, filtered, and concentrated under reduced pressure. The resulting residue was purified by reverse-phase chromatography to provide the desired product (55 mg, 77% yield) as a pale white solid. MS (ESI) m/z=341.2 [M+H]⁺.

Step 3. Synthesis of (trans-3-(4-(6-(4-methylpiperazin-1-yl)pyridin-2-yl)-1H-pyrazol-1-yl)cyclobutyl)methanamine

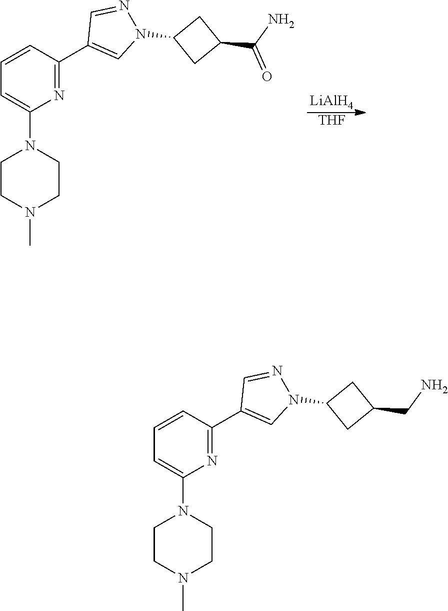 [SMILES: CN1CCN(c2cccc(-c3cnn([C@H]4C[C@H](C(N)=O)C4)c3)n2)CC1 ; "LiAlH4" ; "THF" ; CN1CCN(c2cccc(-c3cnn([C@H]4C[C@H](CN)C4)c3)n2)CC1]

To a solution of methyl 6-(benzyloxy)-2,2-dimethylhexanoate (55.0 mg, 0.16 mmol) in THF (5.0 mL) was added LiAlIH₄ (24.3 mg, 0.6 mmol) at 0° C. The mixture was stirred at reflux under inert atmosphere for 1 h. After cooling down to rt, the reaction mixture was quenched with solid Na₂SO₄ and diluted with EtOAc (15 mL). The suspension was filtered through Celite. The filtrate was concentrated and purified by reverse-phase chromatography to provide the desired product (50.1 mg, 95% yield) as a pale white solid. MS (ESI) m/z=327.2 [M+H]⁺.

Step 4. Synthesis of 2-(2,6-dioxopiperidin-3-yl)-5-(((trans-3-(4-(6-(4-methylpiperazin-1-yl)pyridin-2-yl)-1H-pyrazol-1-yl)cyclobutyl)methyl)amino)isoindoline-1,3-dione

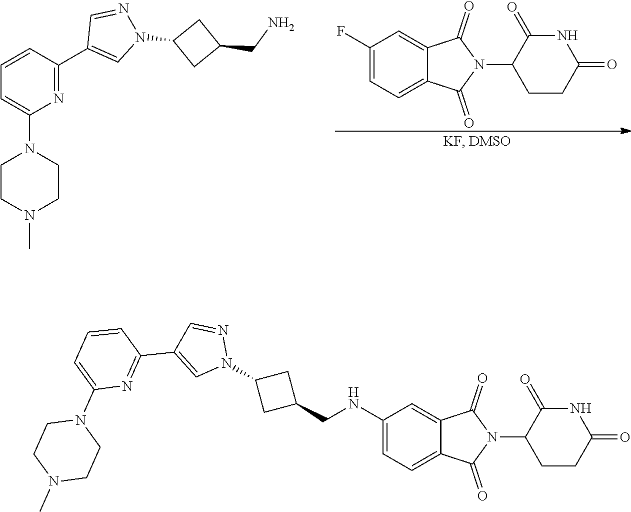 [SMILES: CN1CCN(c2cccc(-c3cnn([C@H]4C[C@H](CN)C4)c3)n2)CC1 ; O=C1CCC(N2C(=O)c3ccc(F)cc3C2=O)C(=O)N1 ; CN1CCN(c2cccc(-c3cnn([C@H]4C[C@H](CNc5ccc6c(c5)C(=O)N(C5CCC(=O)NC5=O)C6=O)C4)c3)n2)CC1]

This step followed the standard procedure for preparing GS-399 to provide the desired product (5.3 mg, 15% yield) as a light white solid. MS (ESI) m/z=583.4 [M+H]⁺.

### Example 375. 2-(2,6-Dioxopiperidin-3-yl)-5-((3-(trans-3-(3-methyl-4-(5-(4-methylpiperazin-1-yl)quinoxalin-2-yl)-1H-pyrazol-1-yl)cyclobutyl)propyl)amino)isoindoline-1,3-dione (GS-417)

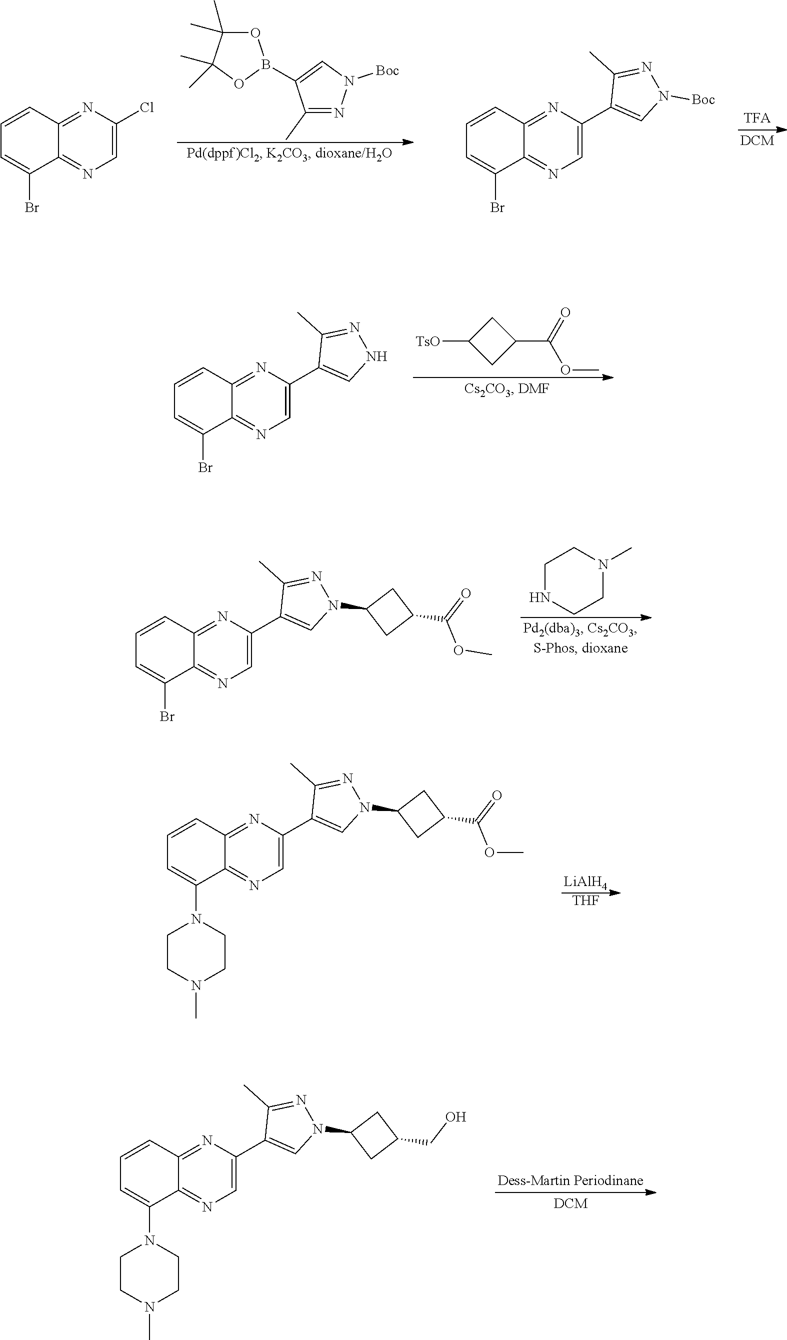

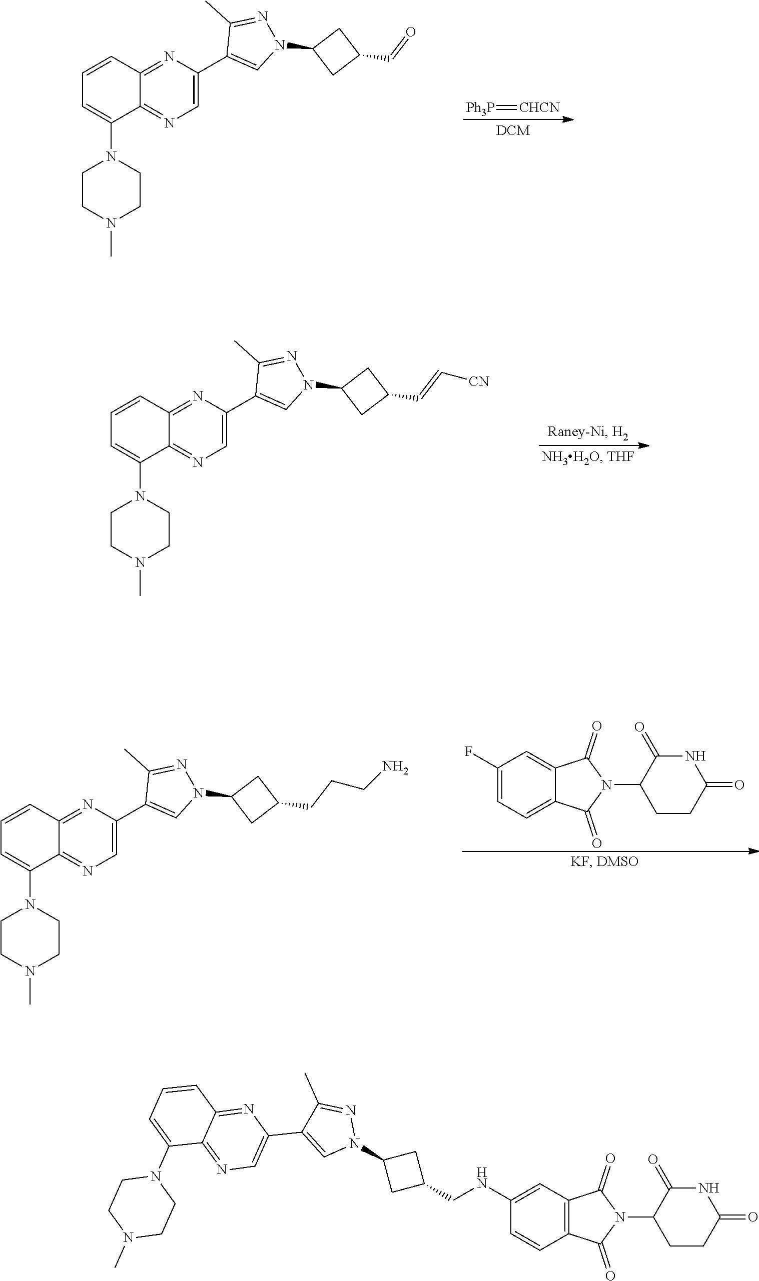

GS-417 was synthesized following the standard procedures for preparing GS-399 (5 mg, 2% yield over 9 steps) as a yellow solid. MS (ESI) m/z=676.4 [M+H]⁺.

### Example 376. 2-(2,6-Dioxopiperidin-3-yl)-5-((3-(trans-3-(3-methyl-4-(5-(1-methylpiperidin-4-yl)quinoxalin-2-yl)-1H-pyrazol-1-yl)cyclobutyl)propyl)amino)isoindoline-1,3-dione (GS-418)

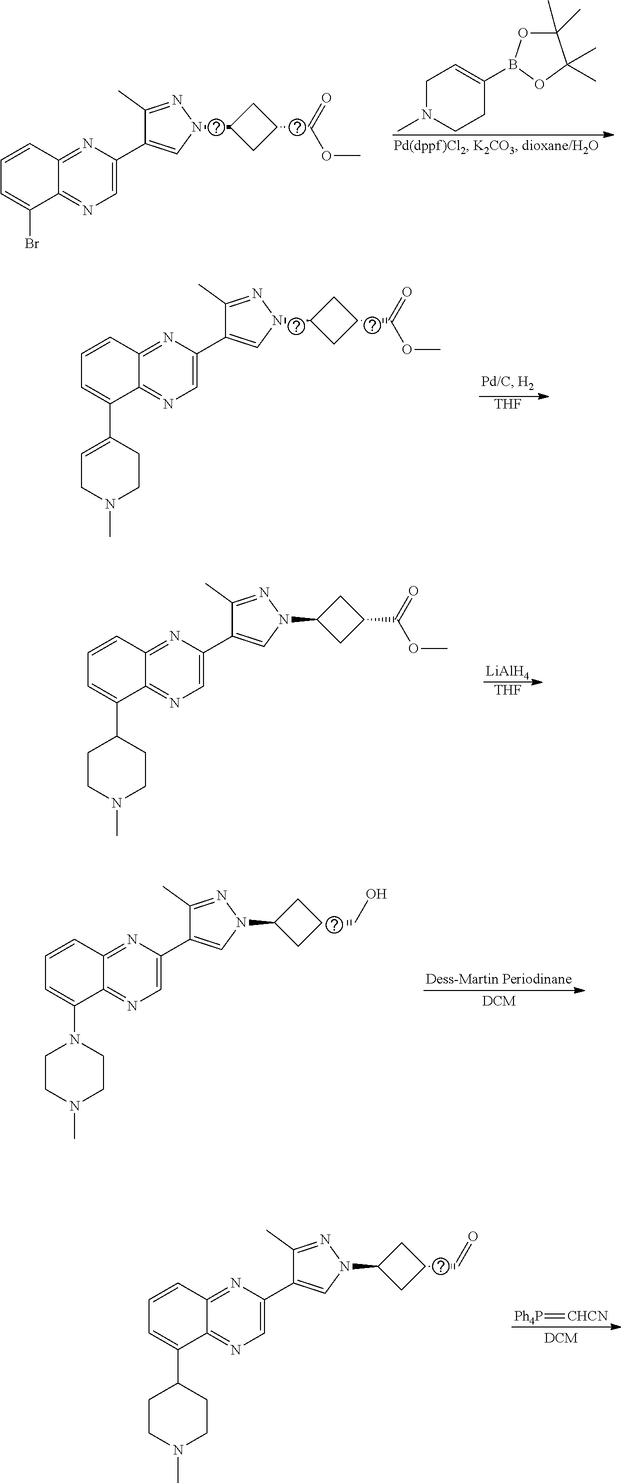

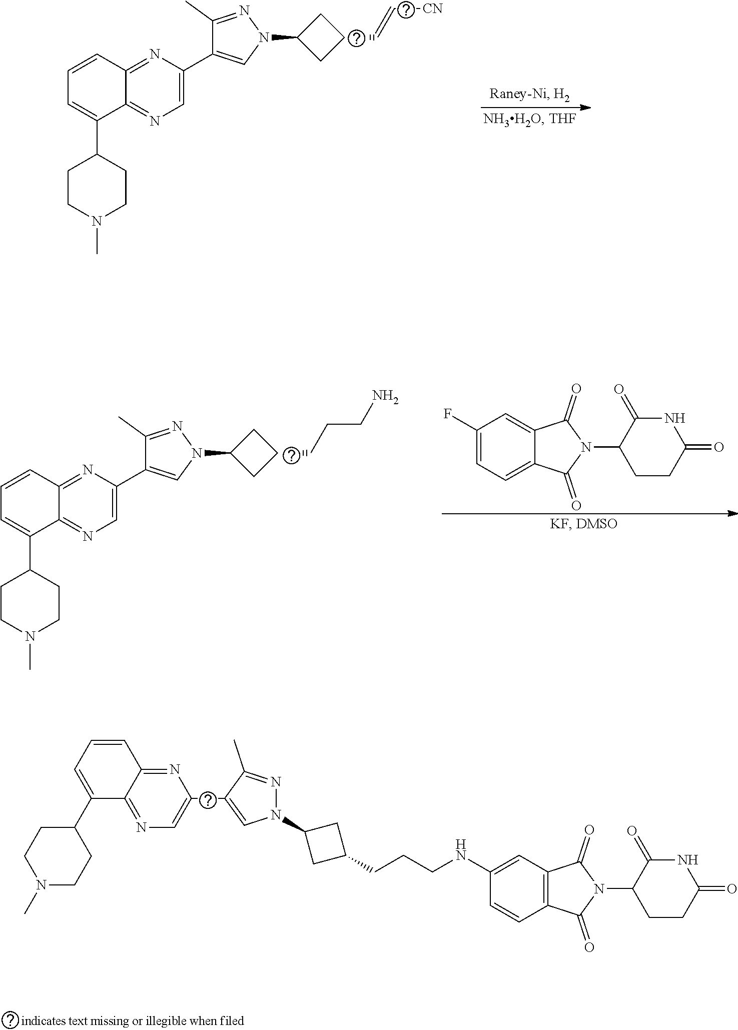 [SMILES: Cc1nn([C@H]2C[C@H](/C=C/C#N)C2)cc1-c1cnc2c(C3CCN(C)CC3)cccc2n1 ; Cc1nn([C@H]2C[C@H](CCCN)C2)cc1-c1cnc2c(C3CCN(C)CC3)cccc2n1 ; O=C1CCC(N2C(=O)c3ccc(F)cc3C2=O)C(=O)N1 ; Cc1nn([C@H]2C[C@H](CCCNc3ccc4c(c3)C(=O)N(C3CCC(=O)NC3=O)C4=O)C2)cc1-c1cnc2c(C3CCN(C)CC3)cccc2n1]

Step 1. Synthesis of methyl trans-3-(3-methyl-4-(5-(1-methyl-1,2,3,6-tetrahydropyridin-4-yl)quinoxalin-2-yl)-1H-pyrazol-1-yl)cyclobutane-1-carboxylate

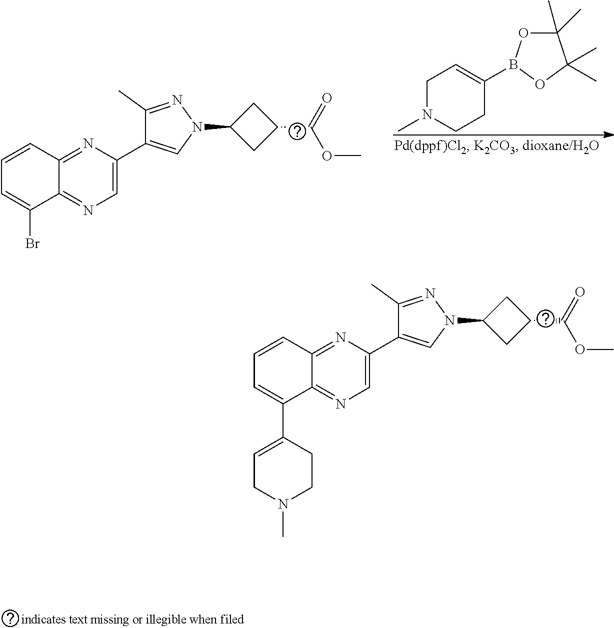

To a solution of methyl trans-3-(4-(5-bromoquinoxalin-2-yl)-3-methyl-1H-pyrazol-1-yl)cyclobutane-1-carboxylate (70 mg, 0.17 mmol) in dioxane (8 mL) and H₂O (2 mL) were added 1-methyl-4-(4,4,5,5-tetramethyl-1,3,2-dioxaborolan-2-yl)-1,2,3,6-tetrahydropyridine (47 mg, 0.21 mmol), K₂CO₃ (70 mg, 0.51 mmol) and Pd(dppf)Cl₂ (13 mg, 0.017 mmol) at rt. The reaction mixture was stirred at 80° C. under nitrogen for 2 h. After cooling down to rt, the mixture was concentrated and purified by reverse-phase chromatography to provide the desired product (60 mg, 82% yield) as a yellow solid. MS (ESI) m/z=418.3 [M+H]⁺.

Step 2. Synthesis of methyl trans-3-(3-methyl-4-(5-(1-methylpiperidin-4-yl)quinoxalin-2-yl)-1H-pyrazol-1-yl)cyclobutane-1-carboxylate

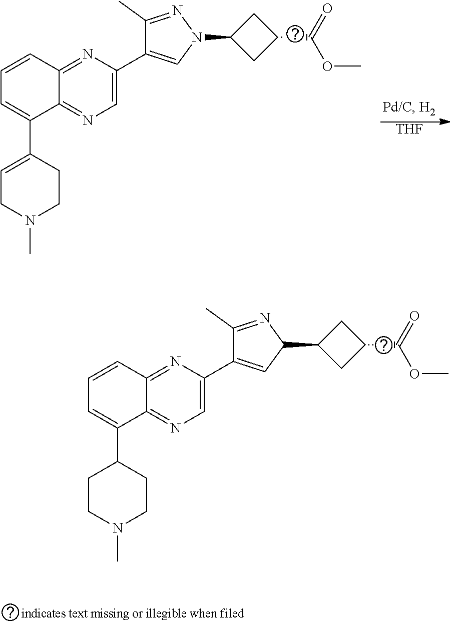 [SMILES: COC(=O)[C@H]1C[C@H](n2cc(-c3cnc4c(C5=CCN(C)CC5)cccc4n3)c(C)n2)C1 ; COC(=O)[C@H]1C[C@H](C2C=C(c3cnc4c(C5CCN(C)CC5)cccc4n3)C(C)=N2)C1 ; "THF"]

A mixture of methyl trans-3-(3-methyl-4-(5-(1-methyl-1,2,3,6-tetrahydropyridin-4-yl)quinoxalin-2-yl)-1H-pyrazol-1-yl)cyclobutane-1-carboxylate (60 mg, 0.14 mmol) and 10% Pd/C (20 mg) in THE (10 mL) was stirred at rt for 2 h under hydrogen balloon. Then the solution was filtered through Celite and the filtrate was evaporated under reduced pressure. The resulting residue was purified by reverse-phase chromatography to provide the desired product (40 mg, 65% yield) as a light-yellow solid. MS (ESI) m/z=420.4 [M+H]⁺.

The remaining steps were performed according to the procedures for preparing GS-399 to provide the desired product (2 mg, 11% yield) as a yellow solid. MS (ESI) m/z=675.5 [M+H]⁺.

### Example 377. 2-(2,6-Dioxopiperidin-3-yl)-5-((3-(trans-3-(4-(6-(1-methylpiperidin-4-yl)quinoxalin-2-yl)-1H-pyrazol-1-yl)cyclobutyl)propyl)amino)isoindoline-1,3-dione (GS-419)

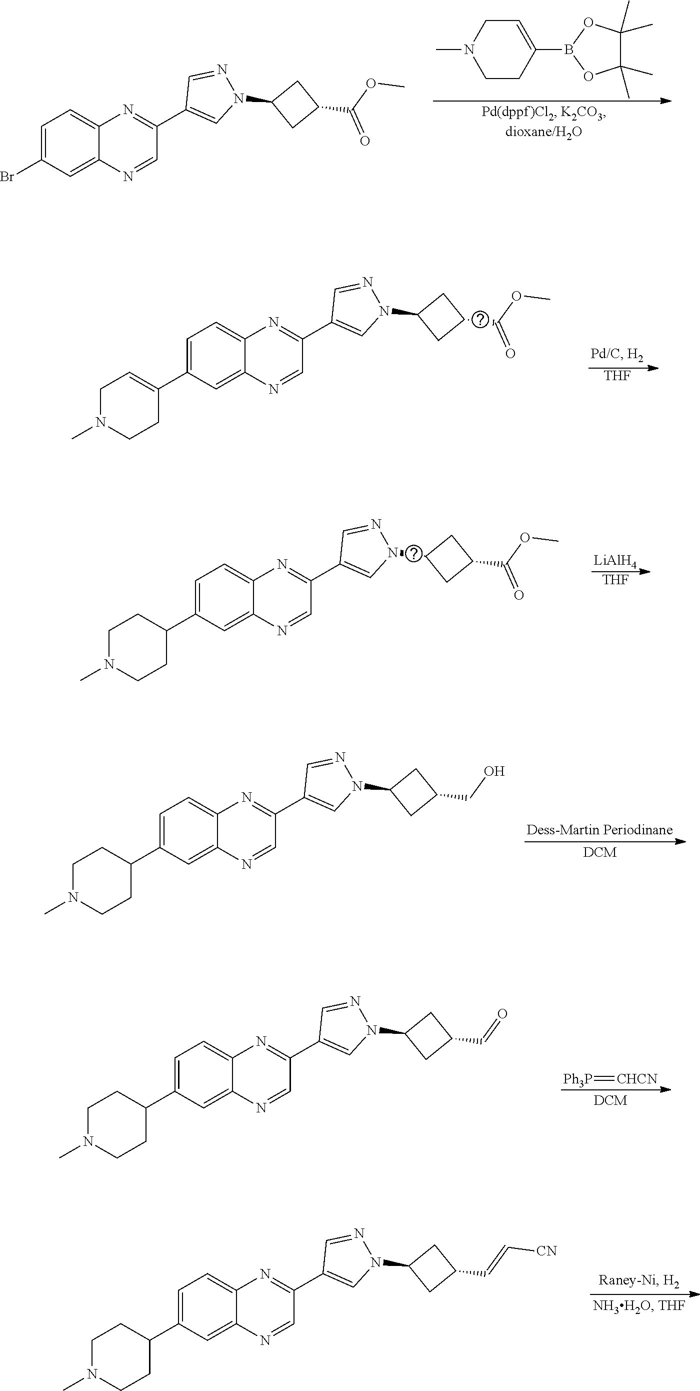

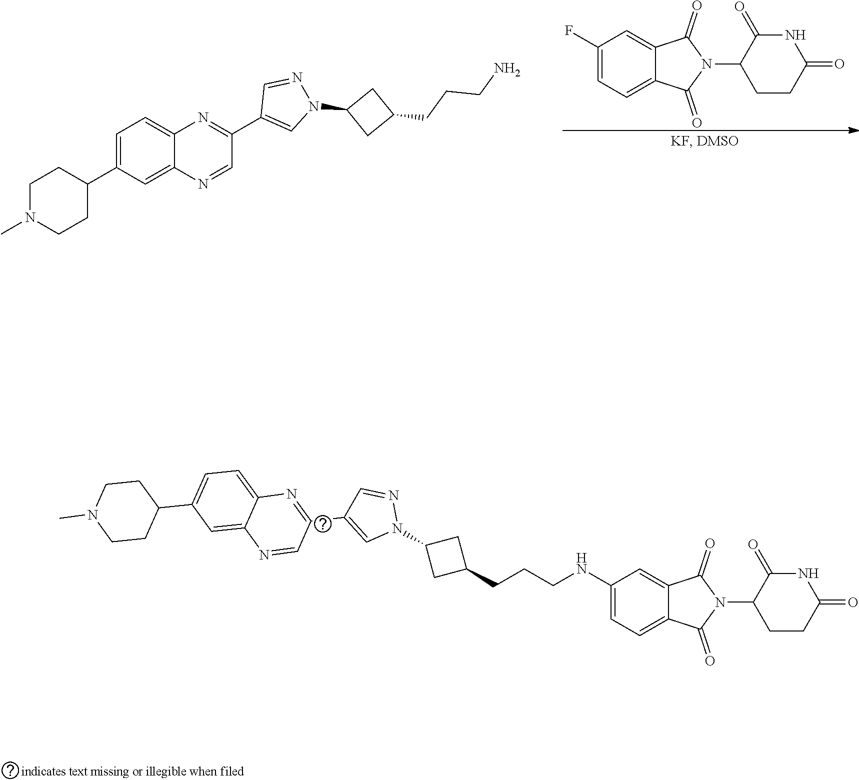 [SMILES: CN1CCC(c2ccc3nc(-c4cnn([C@H]5C[C@H](CCCN)C5)c4)cnc3c2)CC1 ; O=C1CCC(N2C(=O)c3ccc(F)cc3C2=O)C(=O)N1 ; CN1CCC(c2ccc3nc(-c4cnn([C@H]5C[C@H](CCCNc6ccc7c(c6)C(=O)N(C6CCC(=O)NC6=O)C7=O)C5)c4)cnc3c2)CC1]

GS-419 was synthesized following the standard procedures for preparing GS-418 and GS-399 (10 mg, 7% yield over 7 steps) as a yellow solid. MS (ESI) m/z=661.4 [M+H]⁺.

### Example 378. 2-(2,6-Dioxopiperidin-3-yl)-5-((3-(trans-3-(4-(6-(tetrahydro-2H-pyran-4-yl)quinoxalin-2-yl)-1H-pyrazol-1-yl)cyclobutyl)propyl)amino)isoindoline-1,3-dione (GS-420)

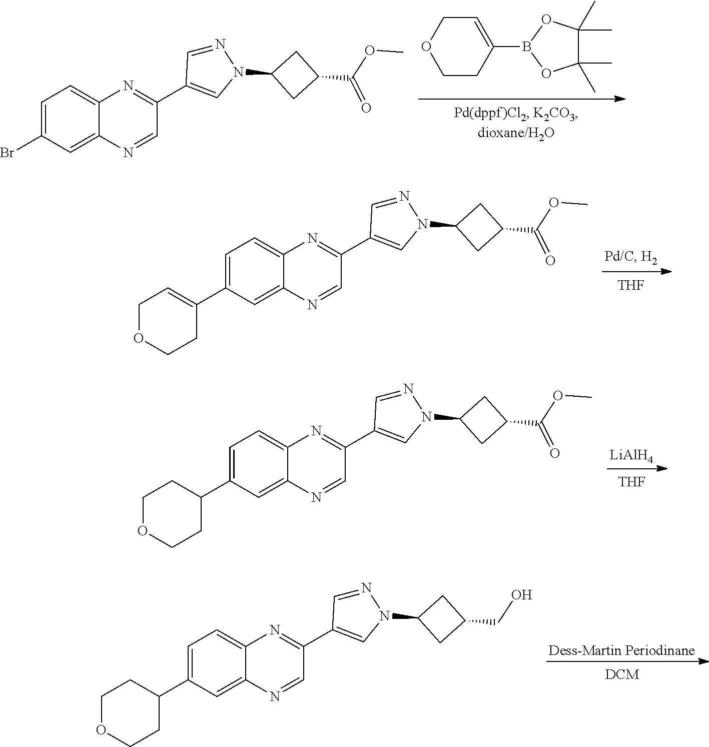

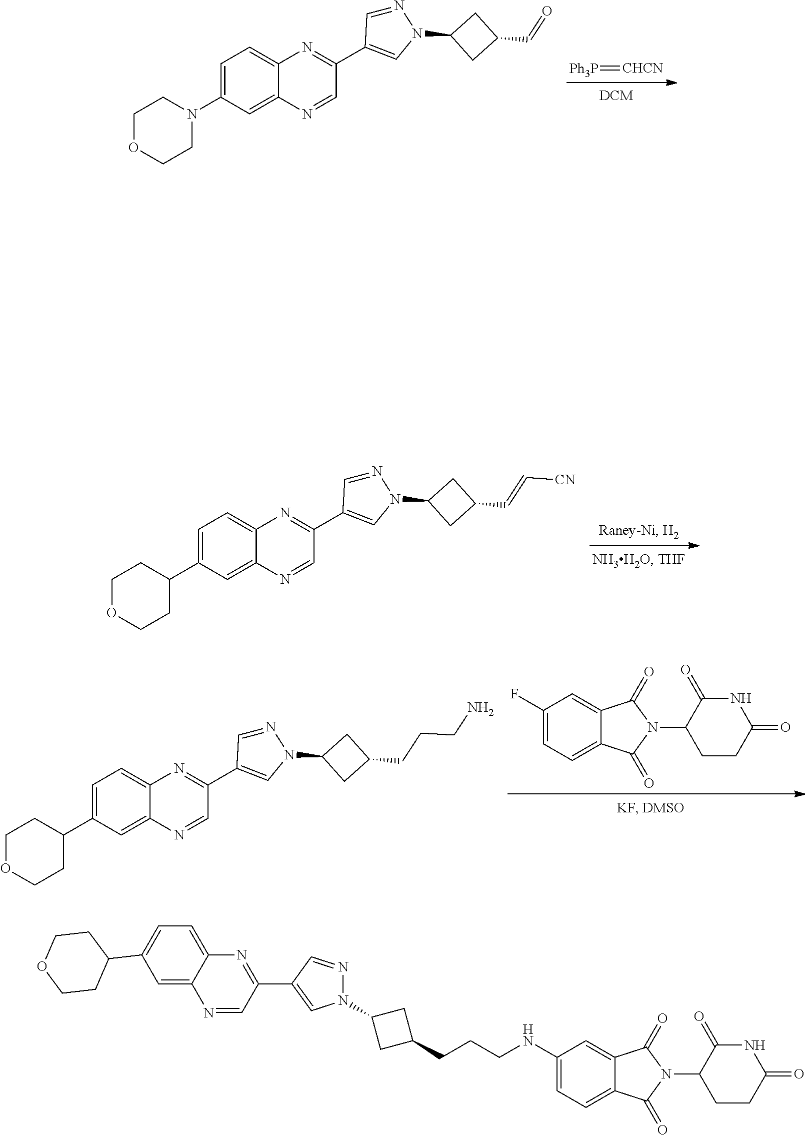

GS-420 was synthesized following the standard procedures for preparing GS-418 and GS-399 (5.5 mg, 11% yield over 7 steps) as a light-yellow solid. MS (ESI) m/z=648.3 [M+H]⁺.

### Example 379. 2-(2,6-Dioxopiperidin-3-yl)-5-((3-(trans-3-(4-(6-(piperidin-4-yl)quinoxalin-2-yl)-1H-pyrazol-1-yl)cyclobutyl)propyl)amino)isoindoline-1,3-dione (GS-421)

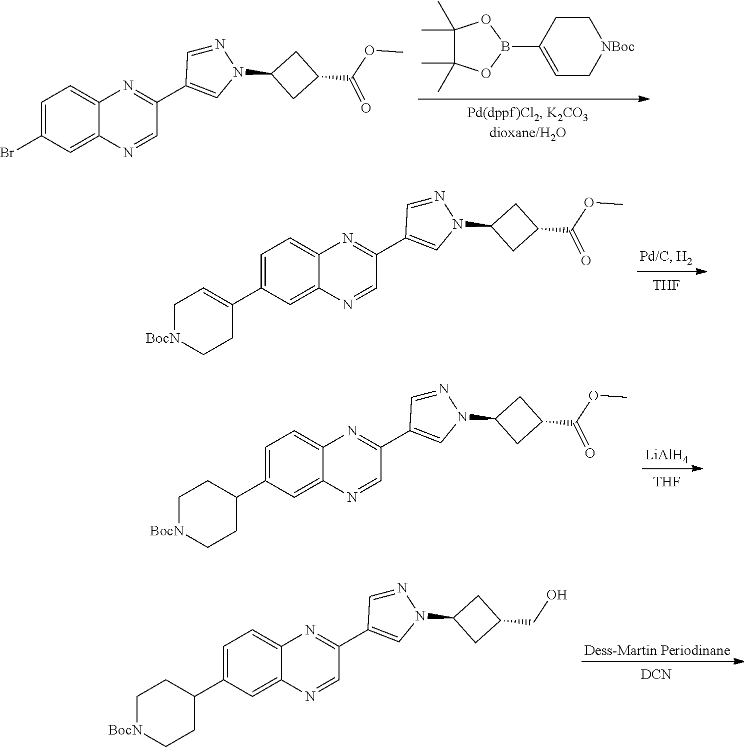

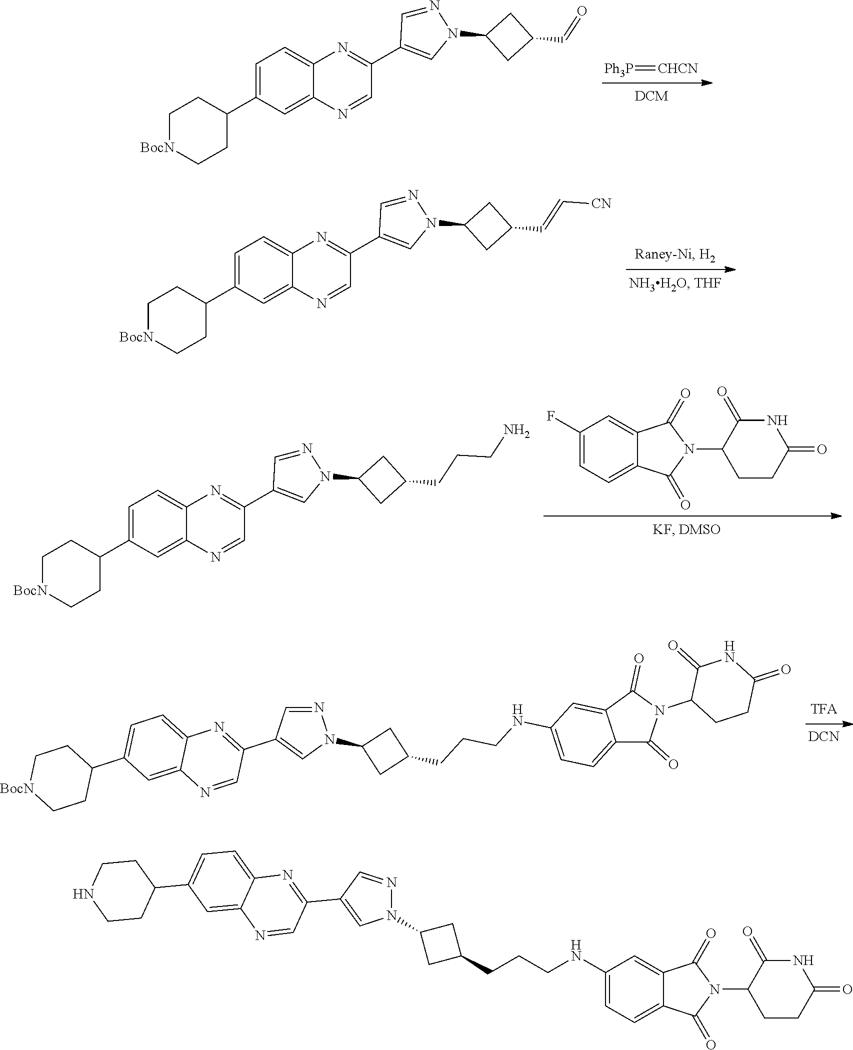

GS-421 was synthesized following the standard procedures for preparing GS-418 and GS-399 (3.1 mg, 2% yield over 8 steps) as a light-yellow solid. MS (ESI) m/z=647.3 [M+H]⁺.

### Example 380. 2-(2,6-Dioxopiperidin-3-yl)-5-((3-(cis-3-(3-methyl-4-(6-morpholinoquinoxalin-2-yl)-1H-pyrazol-1-yl)cyclobutyl)propyl)amino)isoindoline-1,3-dione (GS-422)

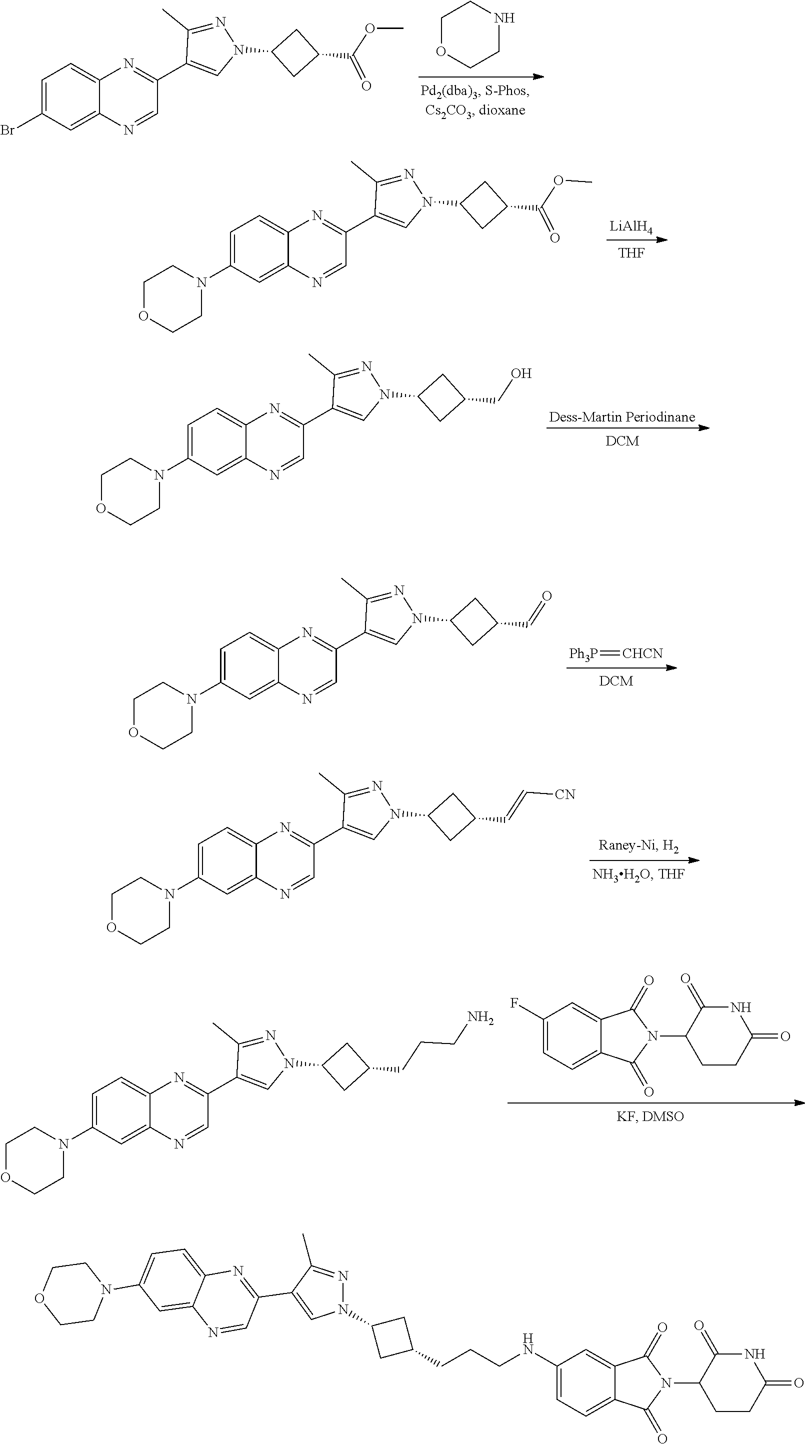

GS-422 was synthesized following the standard procedures for preparing GS-399 (1.1 mg, 1% yield over 6 steps) as a yellow solid. MS (ESI) m/z=663.3 [M+H]⁺.

### Example 381. 2-(2,6-Dioxopiperidin-3-yl)-5-((3-(trans-3-(4-(4-(4-methylpiperazin-1-yl)pyrimidin-2-yl)-1H-pyrazol-1-yl)cyclobutyl)propyl)amino)isoindoline-1,3-dione (GS-423)

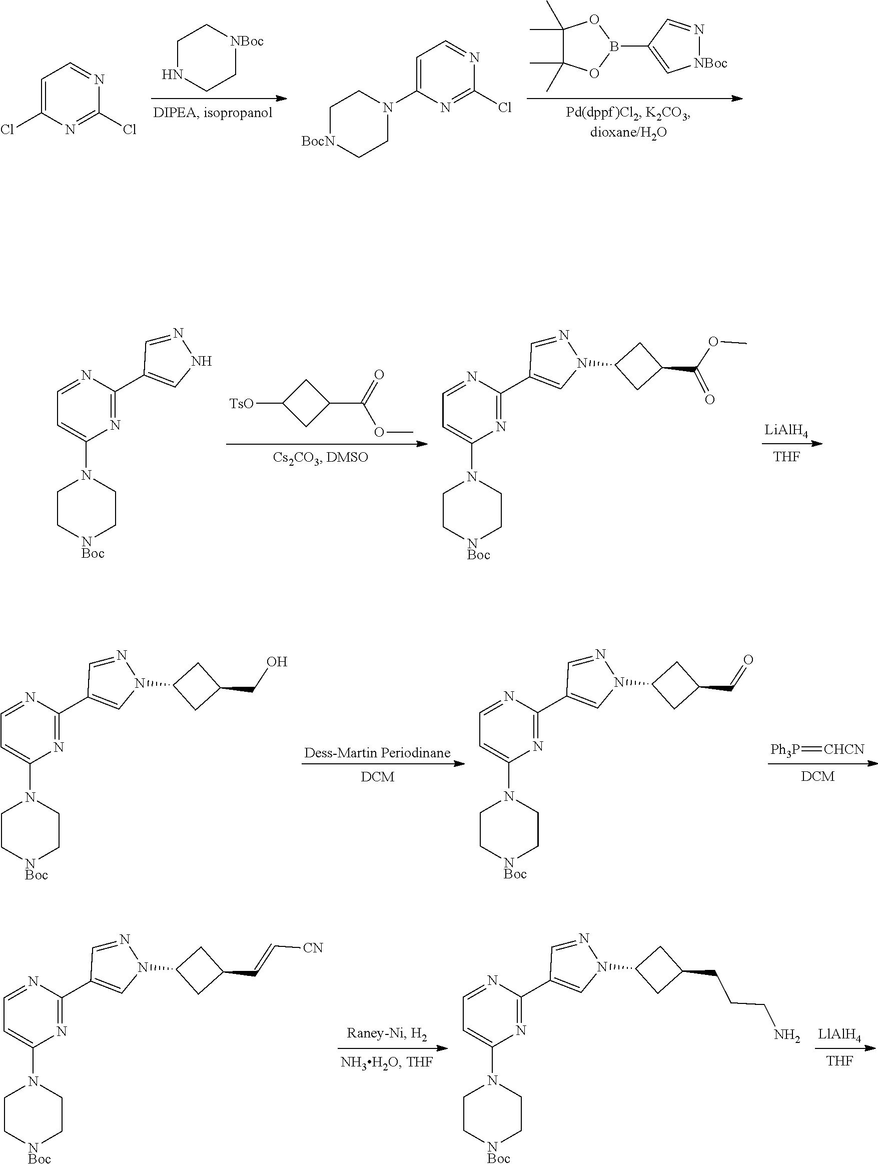

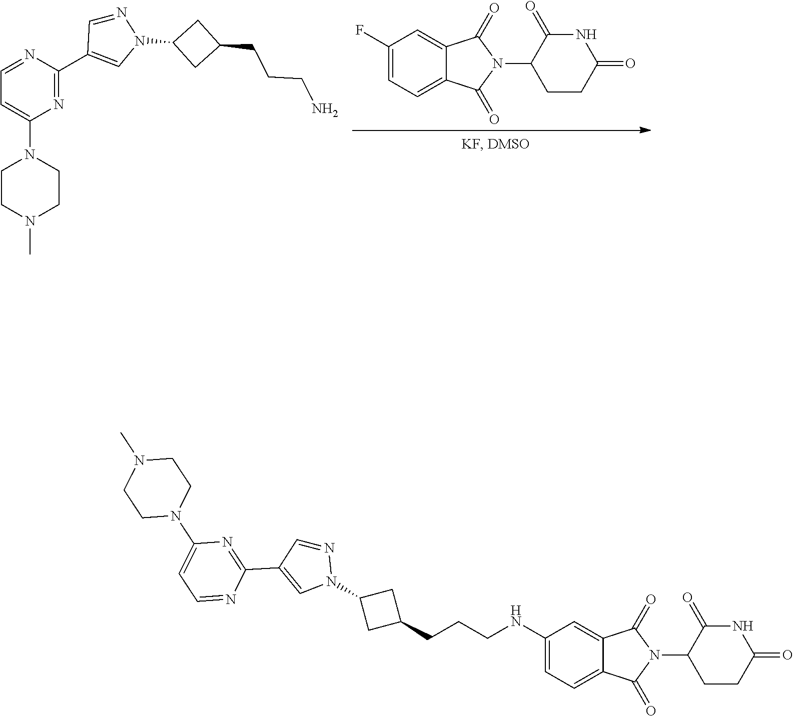 [SMILES: CN1CCN(c2ccnc(-c3cnn([C@H]4C[C@H](CCCN)C4)c3)n2)CC1 ; O=C1CCC(N2C(=O)c3ccc(F)cc3C2=O)C(=O)N1 ; CN1CCN(c2ccnc(-c3cnn([C@H]4C[C@H](CCCNc5ccc6c(c5)C(=O)N(C5CCC(=O)NC5=O)C6=O)C4)c3)n2)CC1]

Step 1. Synthesis of tert-butyl 4-(2-chloropyrimidin-4-yl)piperazine-1-carboxylate

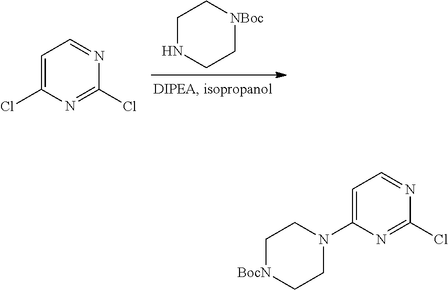 [SMILES: Clc1ccnc(Cl)n1 ; CC(C)(C)OC(=O)N1CCNCC1 ; CC(C)(C)OC(=O)N1CCN(c2ccnc(Cl)n2)CC1]

To a solution of methyl 6-(benzyloxy)-2,2-dimethylhexanoate (50.0 mg, 1.0 mmol) in isopropanol (10.0 mL) were added tert-butyl piperazine-1-carboxylate (279.1 mg, 1.5 mmol) and DIPEA (322.5 mg, 2.5 mmol) at rt. Then the mixture was stirred at 70° C. overnight. After cooling down to rt, the reaction mixture was poured into water (20 mL) and extracted with ethyl acetate (3×20 mL). The combined organic layers were washed with saturated brine (20 mL), dried over anhydrous sodium sulfate, filtered, and concentrated under reduced pressure. The resulting residue was purified by reverse-phase chromatography to provide the desired product (220.0 mg, 74% yield) as a light-yellow solid. MS (ESI) m/z=299.1 [M+H]⁺.

Step 2. Synthesis of tert-butyl 4-(2-(1H-pyrazol-4-yl)pyrimidin-4-yl)piperazine-1-carboxylate

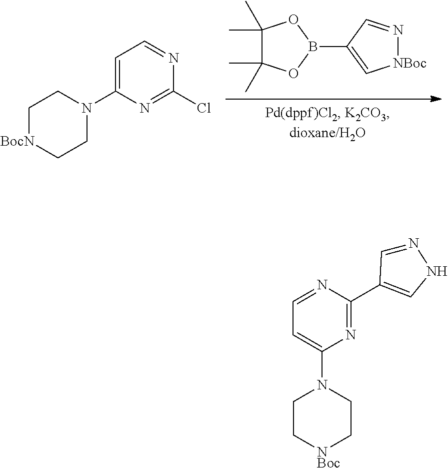

To a solution of tert-butyl 4-(1-chloropyrimidin-4-yl)piperazine-1-carboxylate (122.2 mg, 0.41 mmol) in dioxane (10 mL) and H₂O (2 mL) were added tert-butyl 4-(4,4,5,5-tetramethyl-1,3,2-dioxaborolan-2-yl)-1H-pyrazole-1-carboxylate (151.5 mg, 0.49 mmol), Pd(dppf)Cl₂ (17.9 mg, 2.5 μmol) and K₂CO₃ (113.2 mg, 0.82 mmol) at rt. The mixture was stirred at 100° C. under inert atmosphere for 8 h.

After cooling down to rt, the reaction mixture was poured into water (20 mL) and extracted with ethyl acetate (3×20 mL). The combined organic layers were washed with saturated brine (20 mL), dried over anhydrous sodium sulfate, filtered, and concentrated under reduced pressure. The resulting residue was purified by reverse-phase chromatography to provide the desired product (111.2 mg, 82% yield) as a pale white solid. MS (ESI) m/z=331.2 [M+H]⁺.

Step 3. Synthesis of tert-butyl 4-(2-(1-(trans-3-(methoxycarbonyl)cyclobutyl)-1H-pyrazol-4-yl)pyrimidin-4-yl)piperazine-1-carboxylate

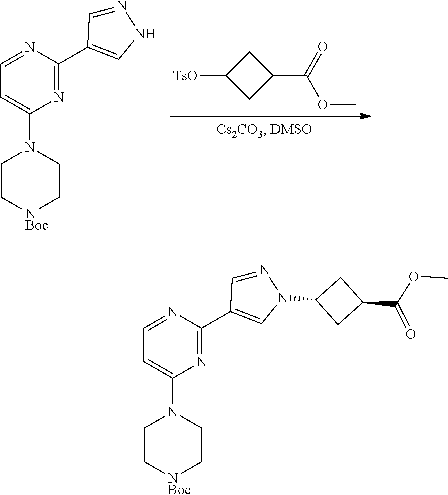 [SMILES: CC(C)(C)OC(=O)N1CCN(c2ccnc(-c3cn[nH]c3)n2)CC1 ; COC(=O)C1CC(OS(=O)(=O)c2ccc(C)cc2)C1 ; COC(=O)[C@H]1C[C@H](n2cc(-c3nccc(N4CCN(C(=O)OC(C)(C)C)CC4)n3)cn2)C1]

To a solution of 6-bromo-2-(1H-pyrazol-4-yl)quinoxaline (330.0 mg, 1.0 mmol) and methyl 3-(tosyloxy)cyclobutane-1-carboxylate (611.0 mg, 1.5 mmol) in DMSO (10 mL) were added Cs₂CO₃ (978.0 mg, 3.0 mmol) and NaI (75.0 mg, 0.5 mmol) at rt. The reaction mixture was stirred at 90° C. for 2 h. After cooling down to rt, the solution was poured into water (50 mL) and extracted with ethyl acetate (3×30 mL). The combined organic layers were washed with saturated brine (30 mL), dried over anhydrous sodium sulfate, filtered, and concentrated under reduced pressure. The resulting residue was purified by silica gel column chromatography to provide two isomers. The desired trans-isomer (160.0 mg, 36% yield) was got as a pale white solid. MS (ESI) m/z=443.2 [M+H]⁺.

The remaining steps were performed according to the procedures for preparing GS-399 to provide the desired product (1.2 mg, 5% yield over 6 steps) as a light-yellow solid. MS (ESI) m/z=612.4 [M+H]⁺.

### Example 382. 2-(2,6-Dioxopiperidin-3-yl)-5-(((trans-3-(4-(4-(4-methylpiperazin-1-yl)pyrimidin-2-yl)-1H-pyrazol-1-yl)cyclobutyl)methyl)amino)isoindoline-1,3-dione (GS-424)

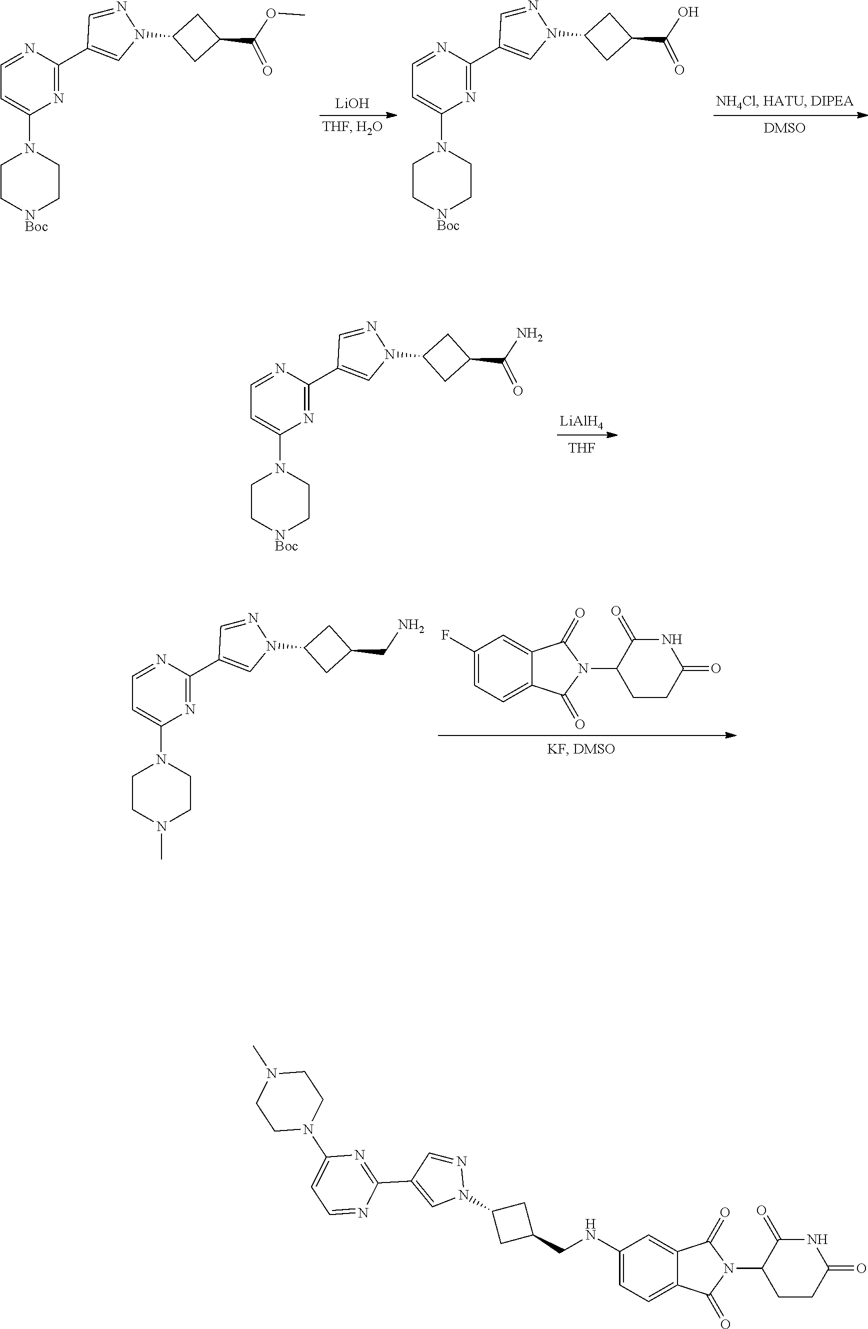

GS-424 was synthesized following the standard procedures for preparing GS-416 (1.6 mg, 8% yield over 4 steps) as a light-yellow solid. MS (ESI) m/z=584.4 [M+H]⁺.

### Example 383. 2-(2,6-Dioxopiperidin-3-yl)-5-((3-(cis-3-(4-(6-(piperazin-1-yl)quinoxalin-2-yl)-1H-pyrazol-1-yl)cyclobutyl)propyl)amino)isoindoline-1,3-dione (GS-425)

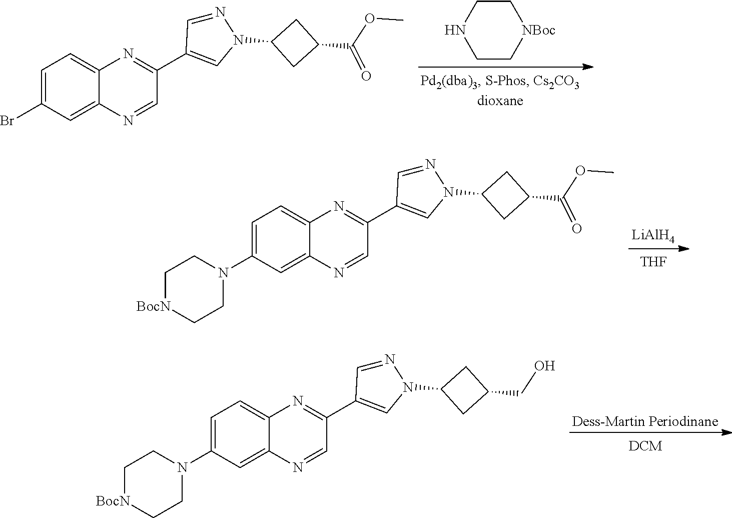

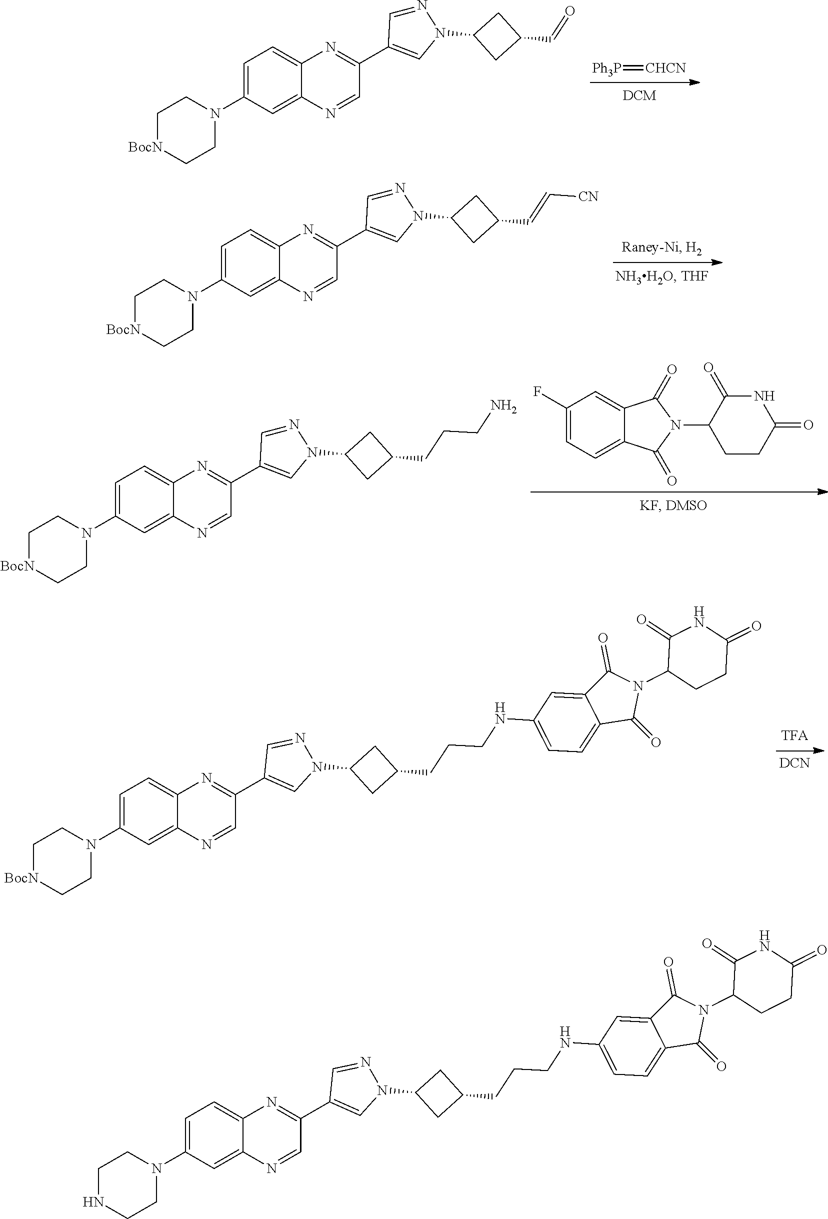

GS-425 was synthesized following the standard procedures for preparing GS-399 (3.2 mg, 4% yield over 7 steps) as a light-yellow solid. MS (ESI) m/z=648.4 [M+H]⁺.

### Example 384. 2-(2,6-Dioxopiperidin-3-yl)-5-((3-(trans-3-(4-(5-(1-methylpiperidin-4-yl)quinoxalin-2-yl)-1H-pyrazol-1-yl)cyclobutyl)propyl)amino)isoindoline-1,3-dione (GS-426)

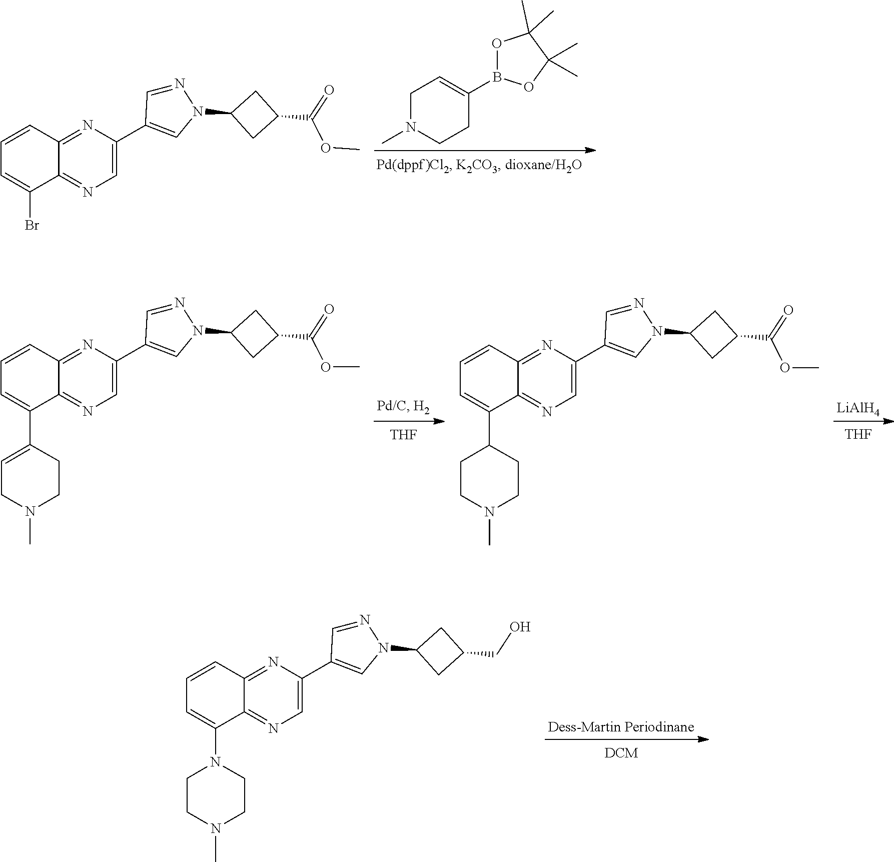

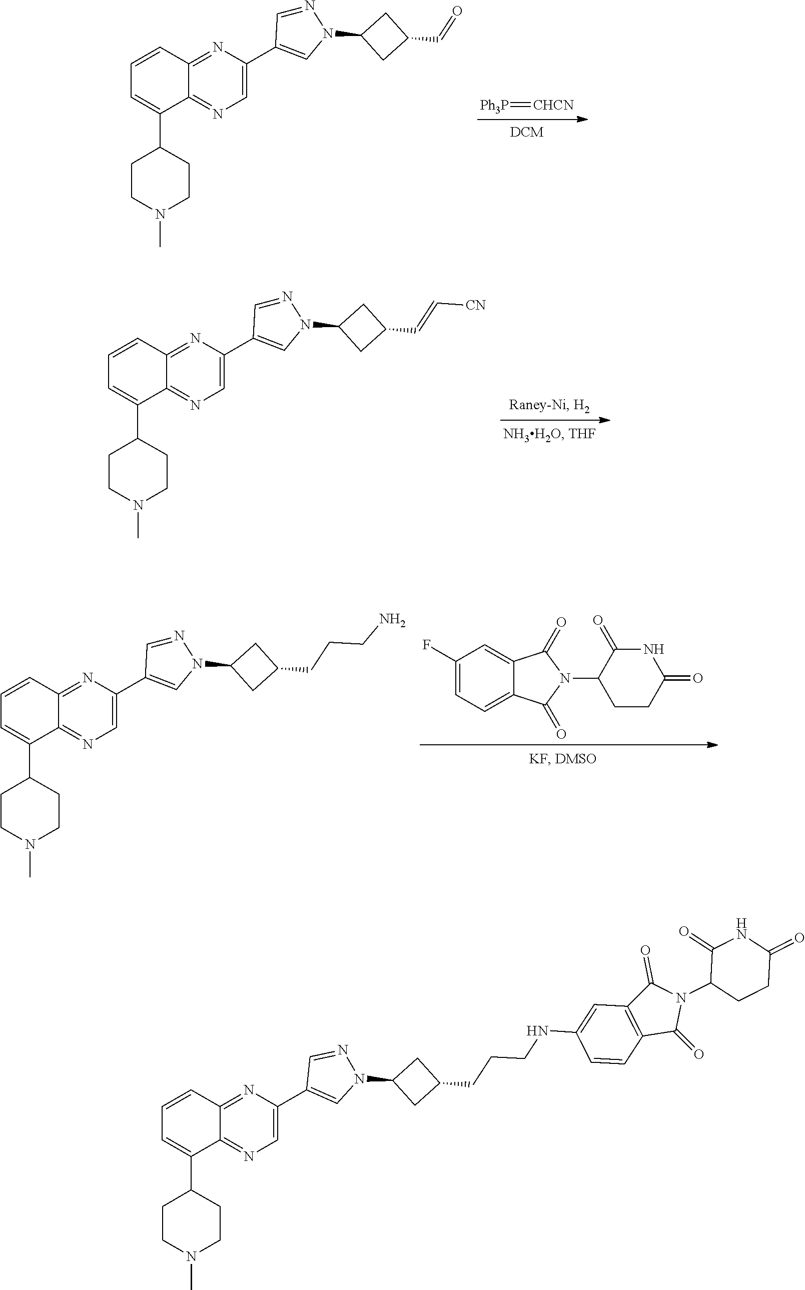

GS-426 was synthesized following the standard procedures for preparing GS-418 and GS-399 (10 mg, 11% yield over 7 steps) as a light-yellow solid. MS (ESI) m/z=661.4 [M+H]⁺.

### Example 385. 2-(2,6-Dioxopiperidin-3-yl)-5-((3-(trans-3-(4-(5-(tetrahydro-2H-pyran-4-yl)quinoxalin-2-yl)-1H-pyrazol-1-yl)cyclobutyl)propyl)amino)isoindoline-1,3-dione (GS-427)

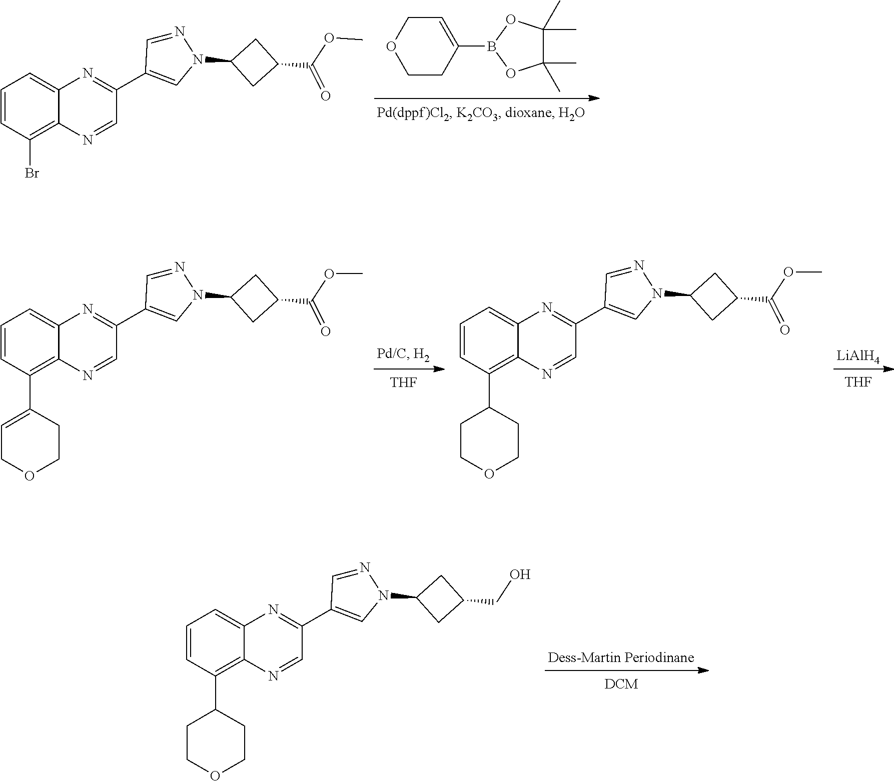

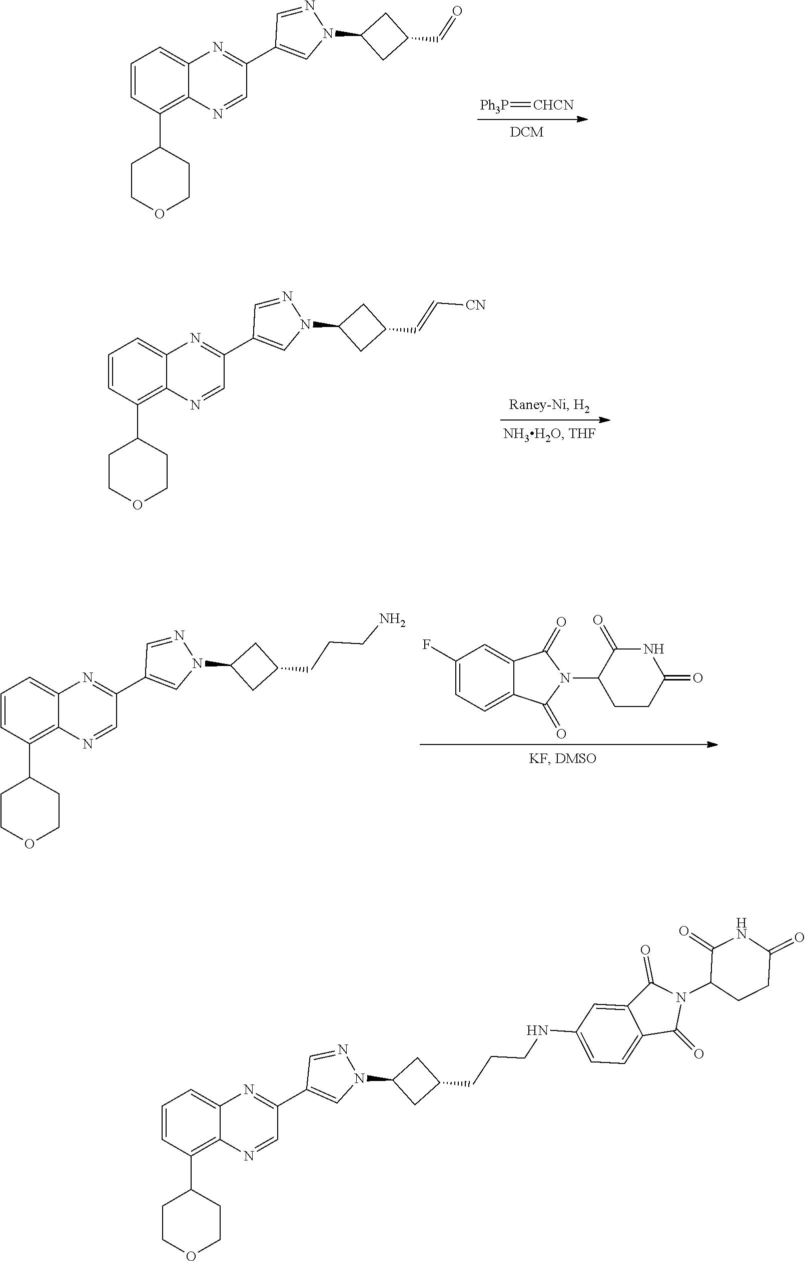

GS-427 was synthesized following the standard procedures for preparing GS-418 and GS-399 (1.8 mg, 3% yield over 7 steps) as a light-yellow solid. MS (ESI) m/z=648.4 [M+H]⁺.

### Example 386. 2-(2,6-Dioxopiperidin-3-yl)-5-((3-(cis-3-(3-methyl-4-(6-(4-methylpiperazin-1-yl)quinoxalin-2-yl)-1H-pyrazol-1-yl)cyclobutyl)propyl)amino)isoindoline-1,3-dione (GS-428)

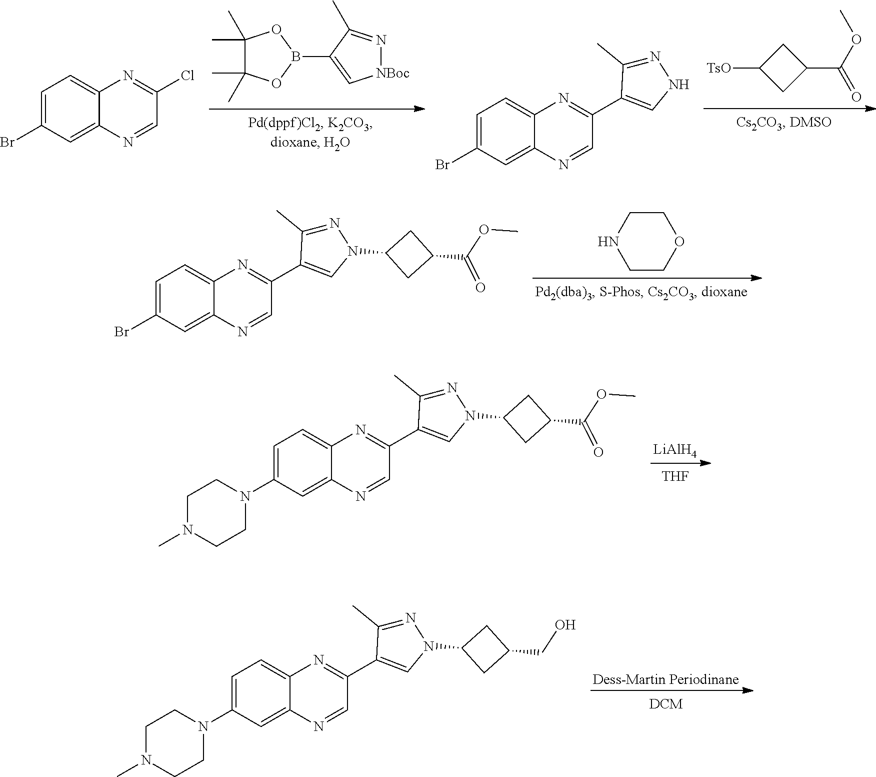

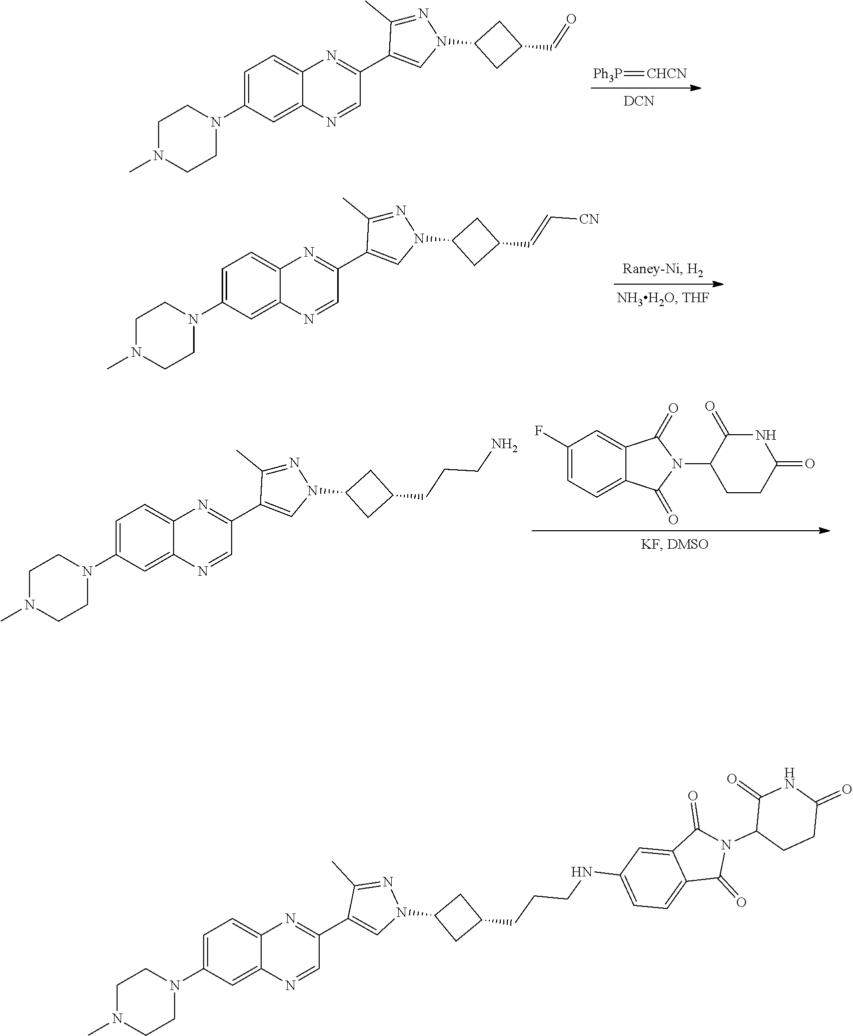

GS-428 was synthesized following the standard procedures for preparing GS-399 (1.3 mg, 12% yield) as a light-yellow solid. MS (ESI) m/z=676.4 [M+H]⁺.

### Example 387. 2-(2,6-Dioxopiperidin-3-yl)-5-((3-(cis-3-(4-(6-(piperidin-4-yl)quinoxalin-2-yl)-1H-pyrazol-1-yl)cyclobutyl)propyl)amino)isoindoline-1,3-dione (GS-429)

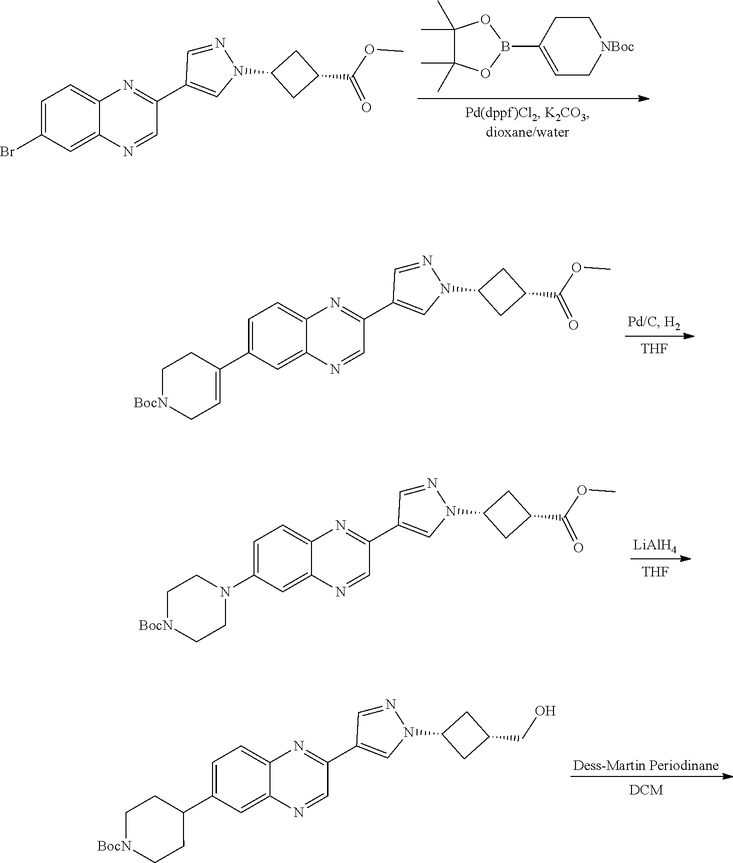

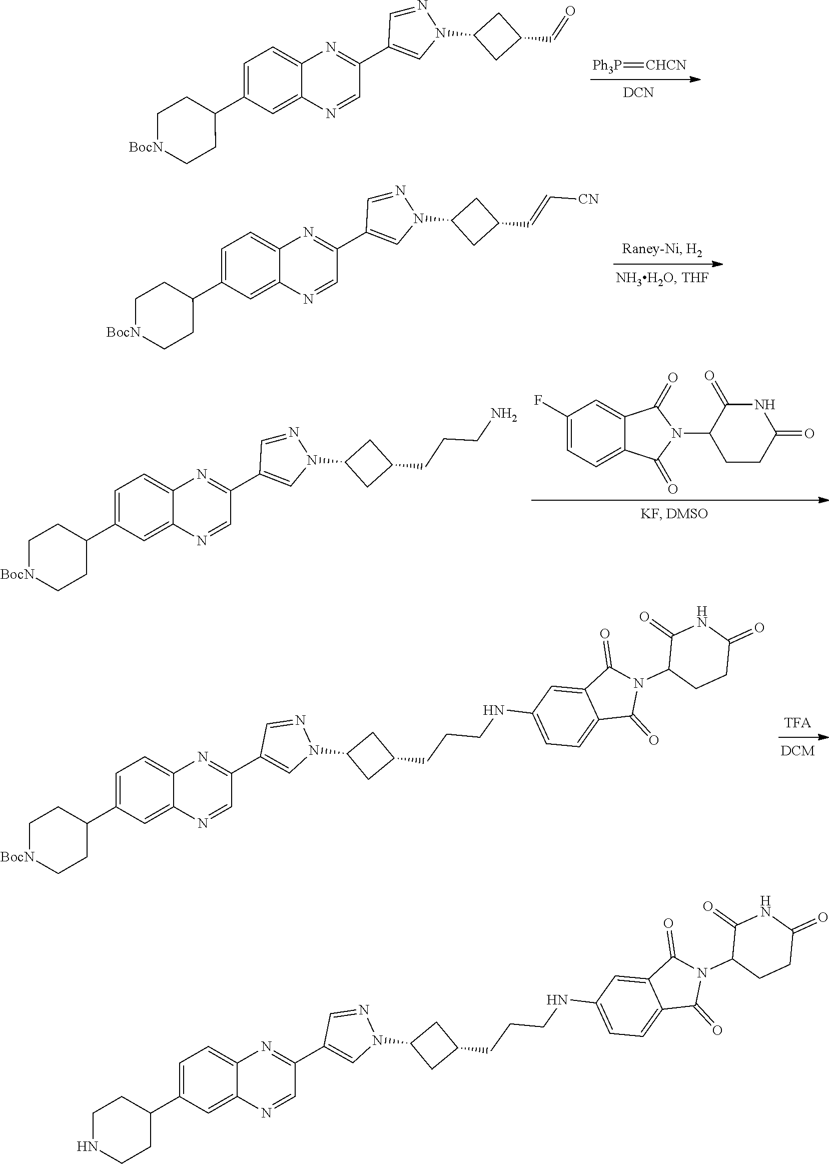

GS-429 was synthesized following the standard procedures for preparing GS-418 and GS-399 (2.6 mg, 8% yield over 8 steps) as a light-yellow solid. MS (ESI) m/z=647.4 [M+H]⁺.

### Example 388. 2-(2,6-Dioxopiperidin-3-yl)-5-((3-(cis-3-(4-(6-(tetrahydro-2H-pyran-4-yl)quinoxalin-2-yl)-1H-pyrazol-1-yl)cyclobutyl)propyl)amino)isoindoline-1,3-dione (GS-430)

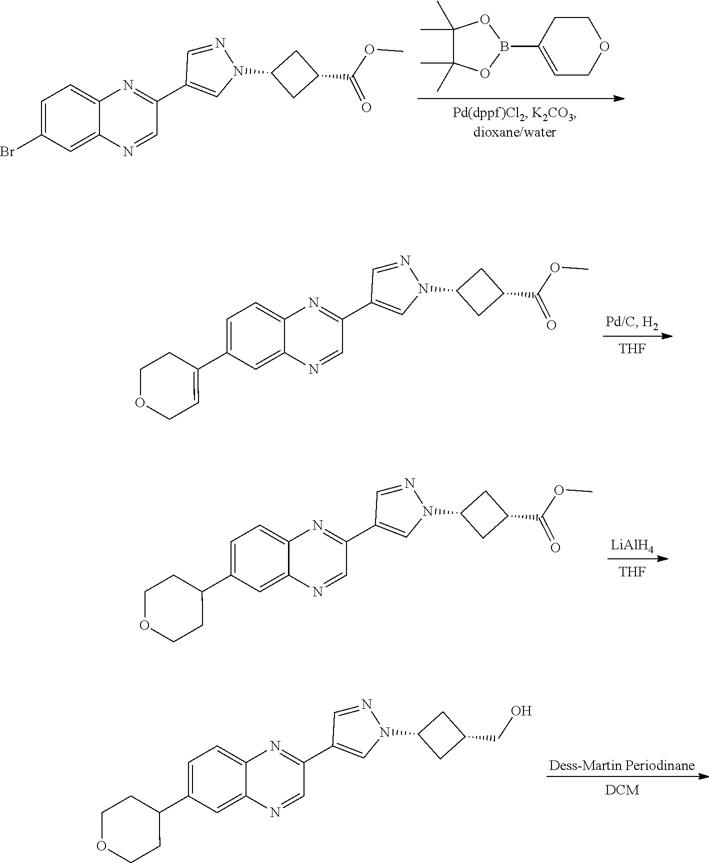

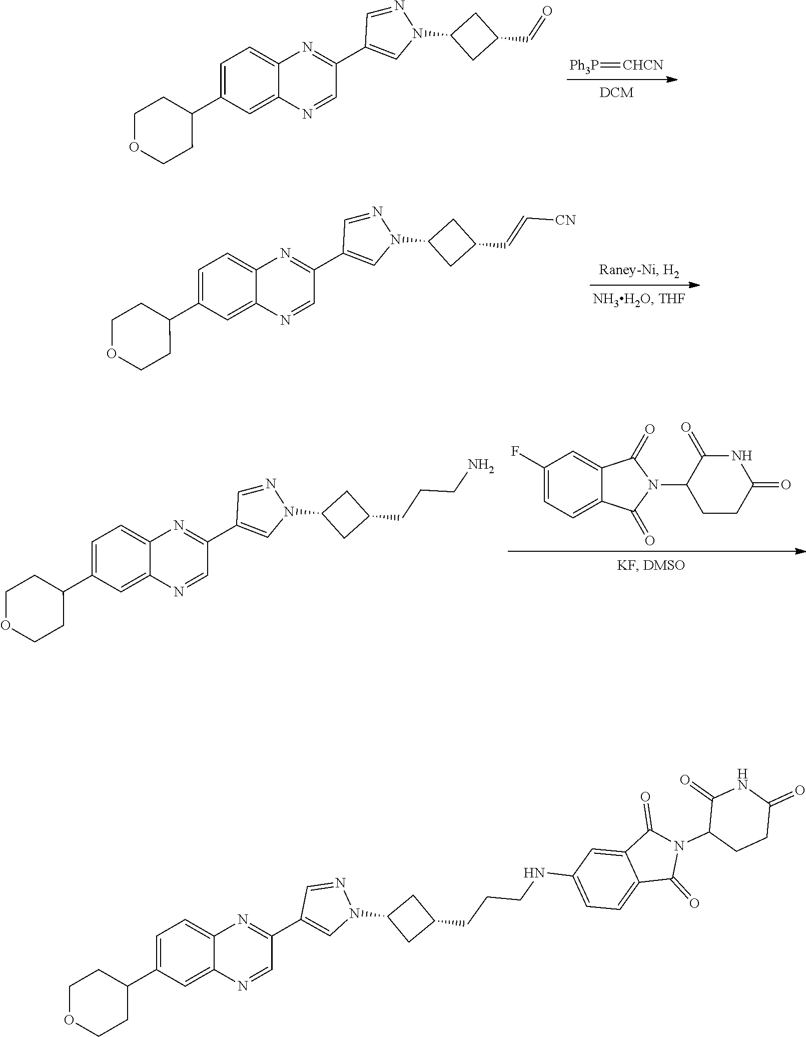

GS-430 was synthesized following the standard procedures for preparing GS-418 and GS-399 (5.5 mg, 11% yield over 7 steps) as a light-yellow solid. MS (ESI) m/z=648.3 [M+H]⁺.

### Example 389. 2-(2,6-Dioxopiperidin-3-yl)-5-((3-(trans-3-(4-(5-(piperidin-4-yl)quinoxalin-2-yl)-1H-pyrazol-1-yl)cyclobutyl)propyl)amino)isoindoline-1,3-dione (GS-431)

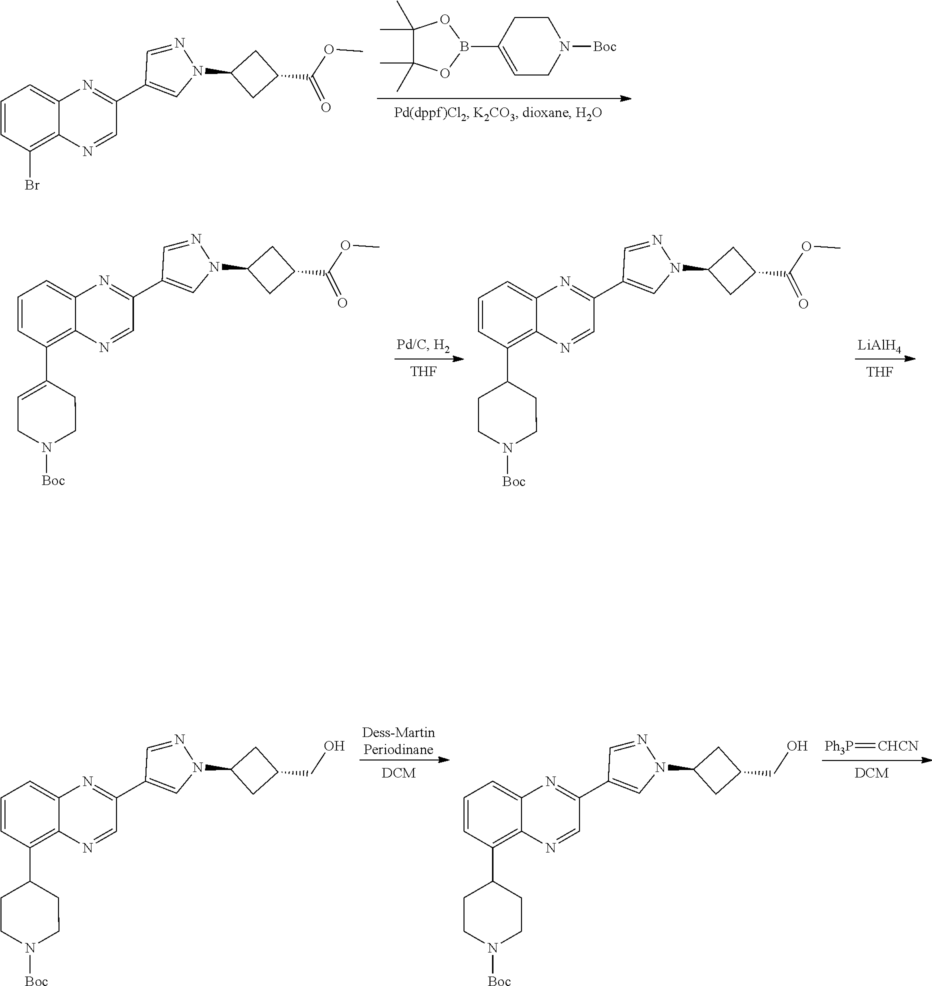

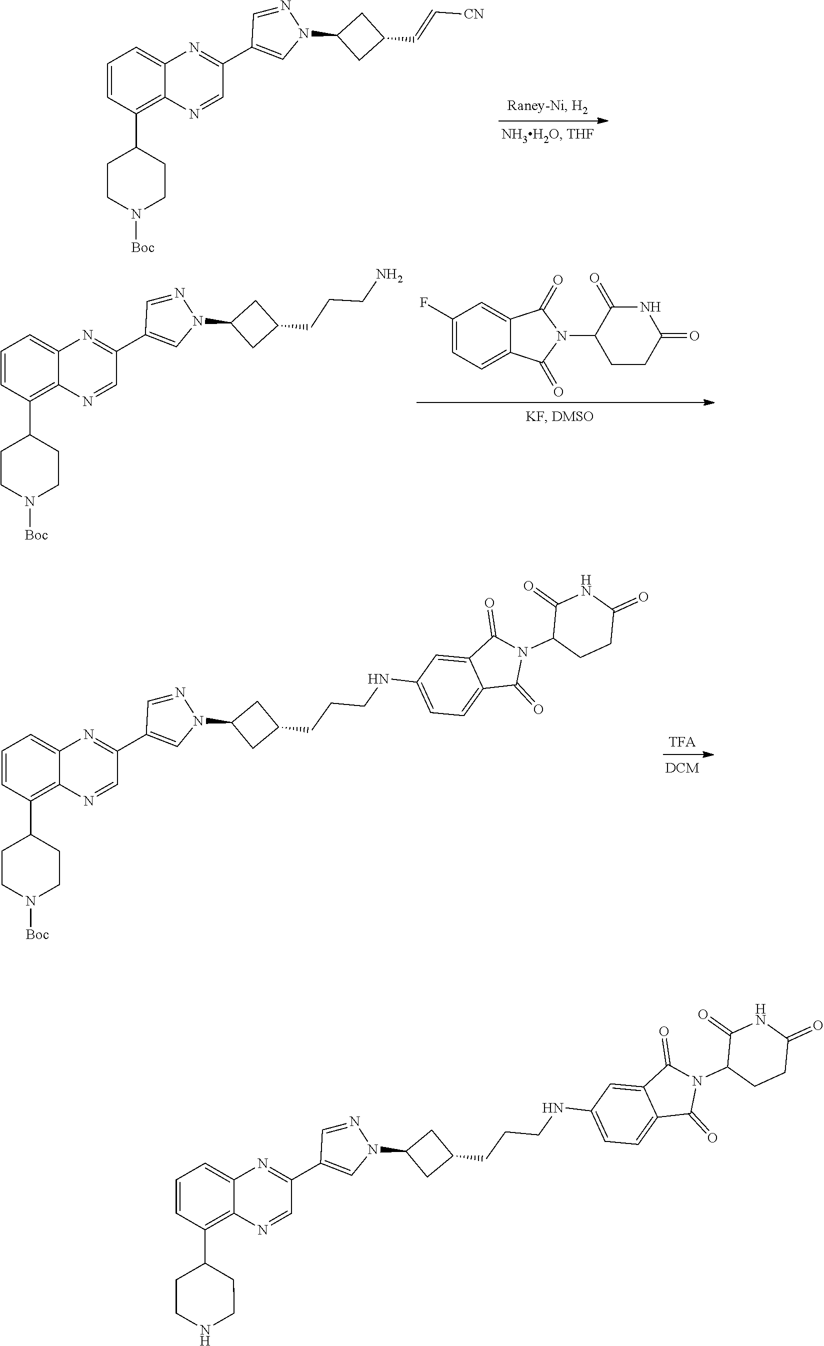 [SMILES: CC(C)(C)OC(=O)N1CCC(c2cccc3nc(-c4cnn([C@H]5C[C@H](/C=C/C#N)C5)c4)cnc23)CC1 ; CC(C)(C)OC(=O)N1CCC(c2cccc3nc(-c4cnn([C@H]5C[C@H](CCCN)C5)c4)cnc23)CC1 ; O=C1CCC(N2C(=O)c3ccc(F)cc3C2=O)C(=O)N1 ; CC(C)(C)OC(=O)N1CCC(c2cccc3nc(-c4cnn([C@H]5C[C@H](CCCNc6ccc7c(c6)C(=O)N(C6CCC(=O)NC6=O)C7=O)C5)c4)cnc23)CC1 ; O=C1CCC(N2C(=O)c3ccc(NCCC[C@H]4C[C@H](n5cc(-c6cnc7c(C8CCNCC8)cccc7n6)cn5)C4)cc3C2=O)C(=O)N1]

GS-431 was synthesized following the standard procedures for preparing GS-418 and GS-399 (1.3 mg, 3% yield over 8 steps) as a yellow solid. MS (ESI) m/z=647.3 [M+H]⁺.

### Example 390. 2-(2,6-Dioxopiperidin-3-yl)-5-((3-(cis-3-(4-(6-(1-methylpiperidin-4-yl)quinoxalin-2-yl)-1H-pyrazol-1-yl)cyclobutyl)propyl)amino)isoindoline-1,3-dione (GS-432)

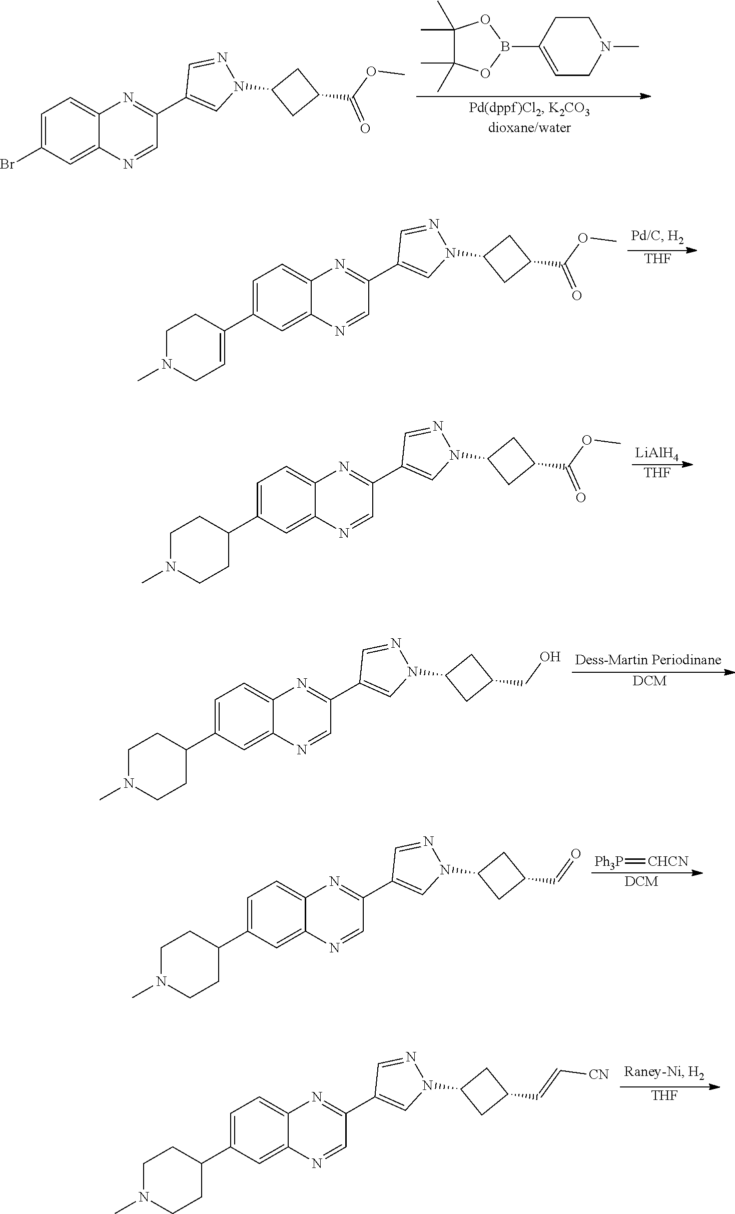

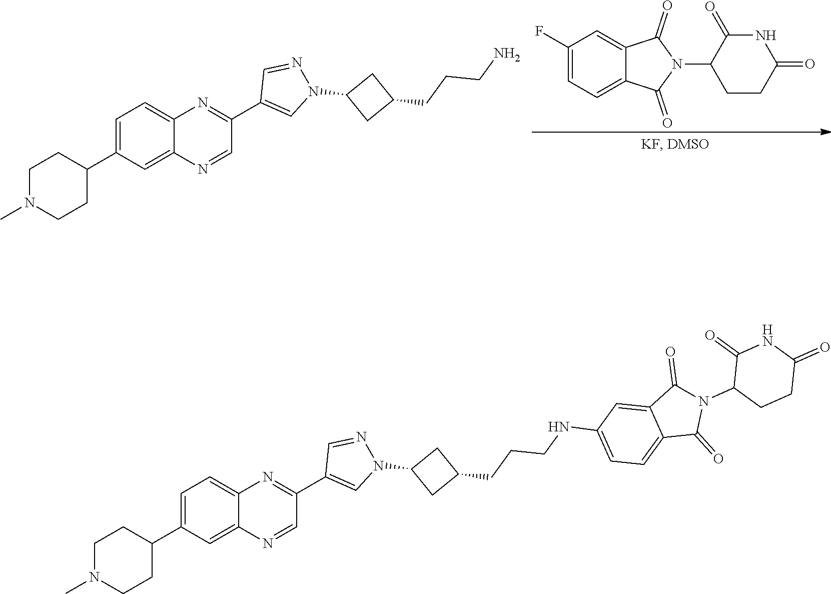

GS-432 was synthesized following the standard procedures for preparing GS-418 and GS-399 (3.0 mg, 6% yield over 7 steps) as a light-yellow solid. MS (ESI) m/z=661.4 [M+H]⁺.

### Example 391. 2-(2,6-Dioxopiperidin-3-yl)-5-((3-(cis-3-(3-methyl-4-(6-morpholinoquinoxalin-2-yl)-1H-pyrazol-1-yl)cyclobutyl)propyl)amino)isoindoline-1,3-dione (GS-433)

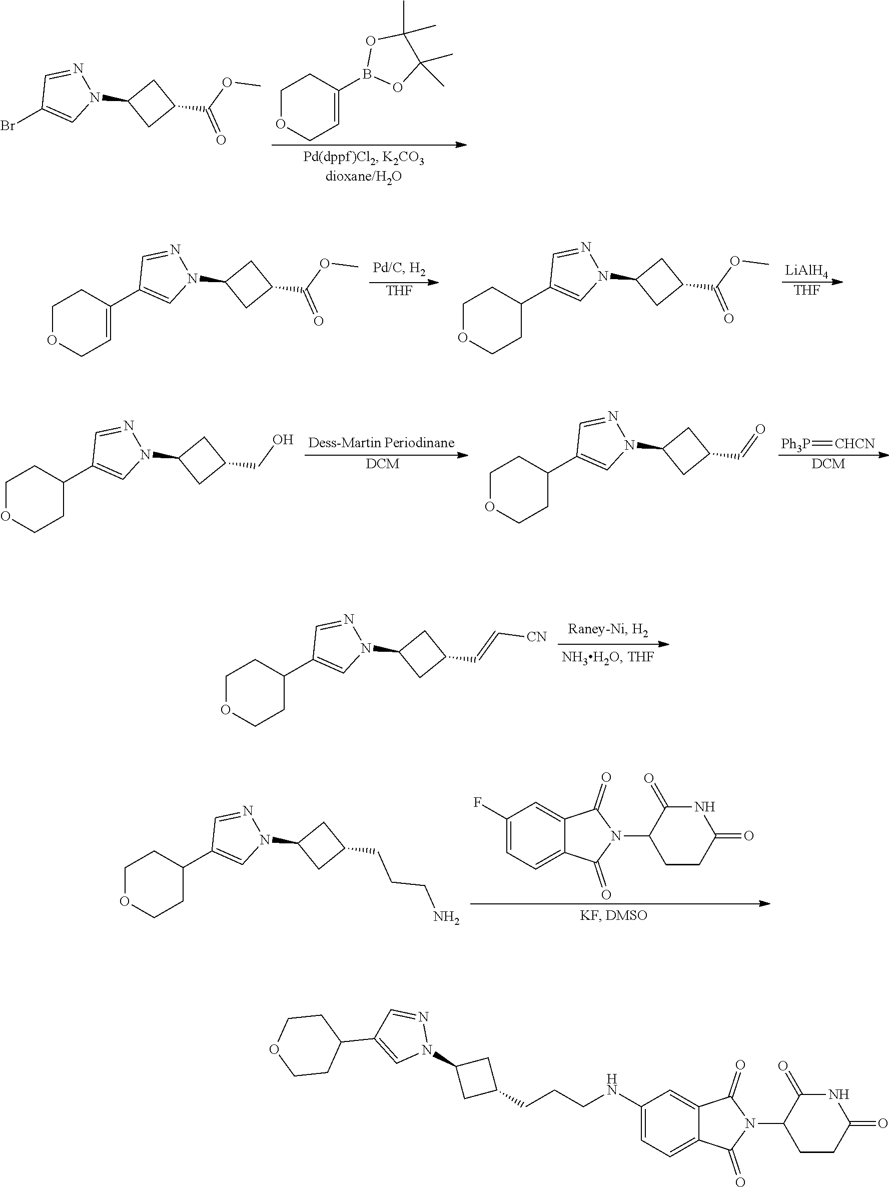

GS-433 was synthesized following the standard procedures for preparing GS-418 and GS-399 (2.6 mg, 1% yield over 7 steps) as a light-yellow solid. MS (ESI) m/z=520.3 [M+H]⁺.

### Example 392. 2-(2,6-Dioxopiperidin-3-yl)-5-((3-(cis-3-(3-methyl-4-(6-(piperazin-1-yl)quinoxalin-2-yl)-1H-pyrazol-1-yl)cyclobutyl)propyl)amino)isoindoline-1,3-dione (GS-434)

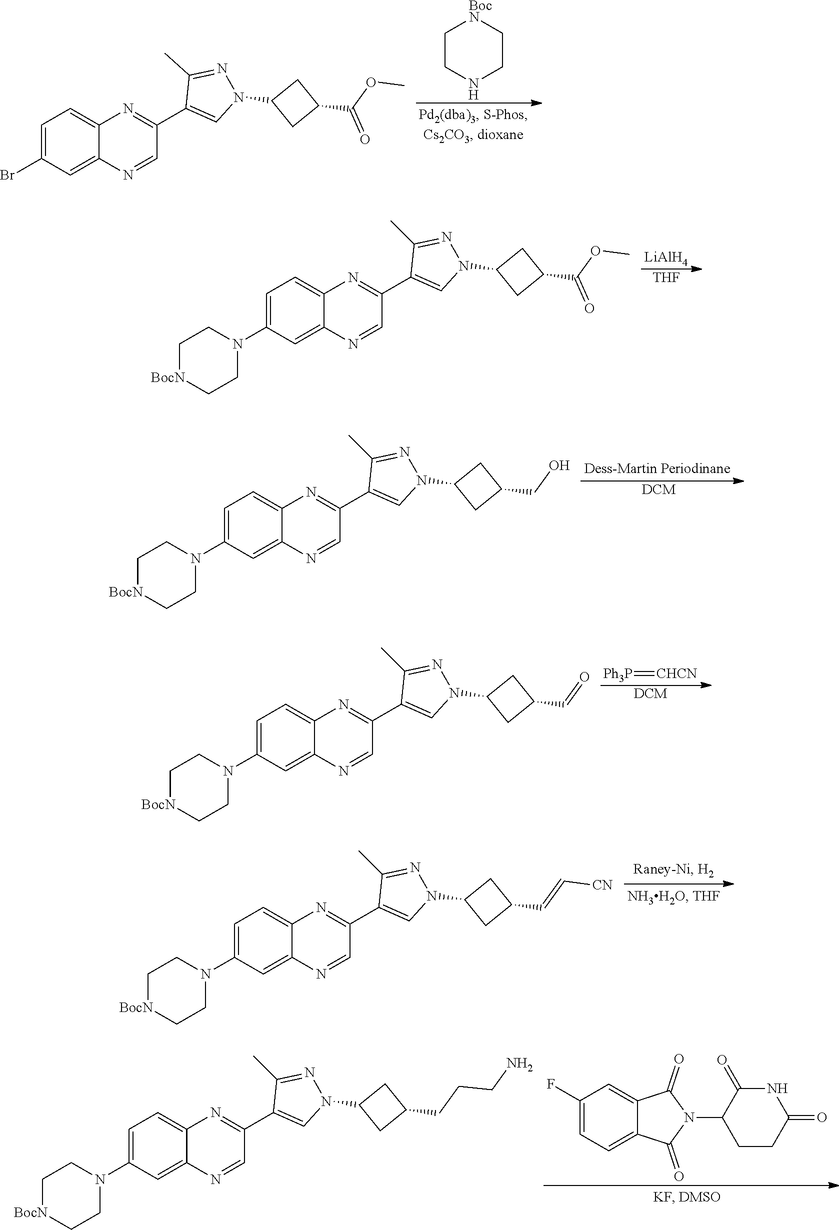

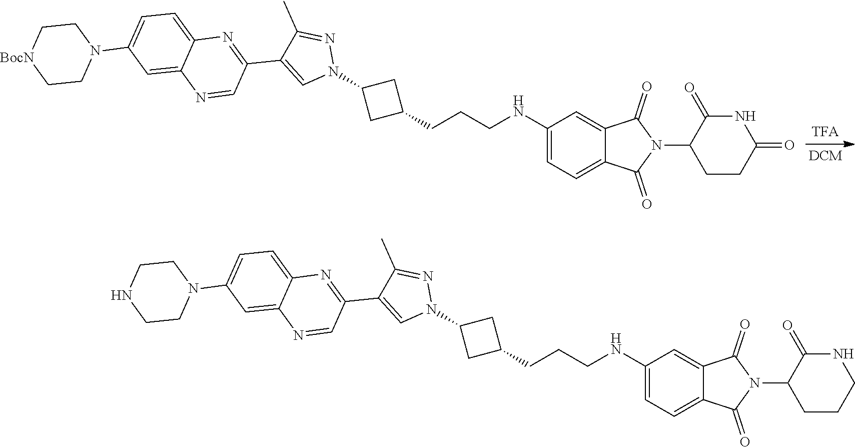 [SMILES: Cc1nn([C@H]2C[C@@H](CCCNc3ccc4c(c3)C(=O)N(C3CCC(=O)NC3=O)C4=O)C2)cc1-c1cnc2cc(N3CCN(C(=O)OC(C)(C)C)CC3)ccc2n1 ; Cc1nn([C@H]2C[C@@H](CCCNc3ccc4c(c3)C(=O)N(C3CCCNC3=O)C4=O)C2)cc1-c1cnc2cc(N3CCNCC3)ccc2n1]

GS-434 was synthesized following the standard procedures for preparing GS-399 (1.4 mg, 6% yield over 7 steps) as a light white solid. MS (ESI) m/z=662.4 [M+H]⁺.

### Example 393. 2-(2,6-Dioxopiperidin-3-yl)-5-((3-(trans-3-(4-(1-methylpiperidin-4-yl)-1H-pyrazol-1-yl)cyclobutyl)propyl)amino)isoindoline-1,3-dione (GS-435)

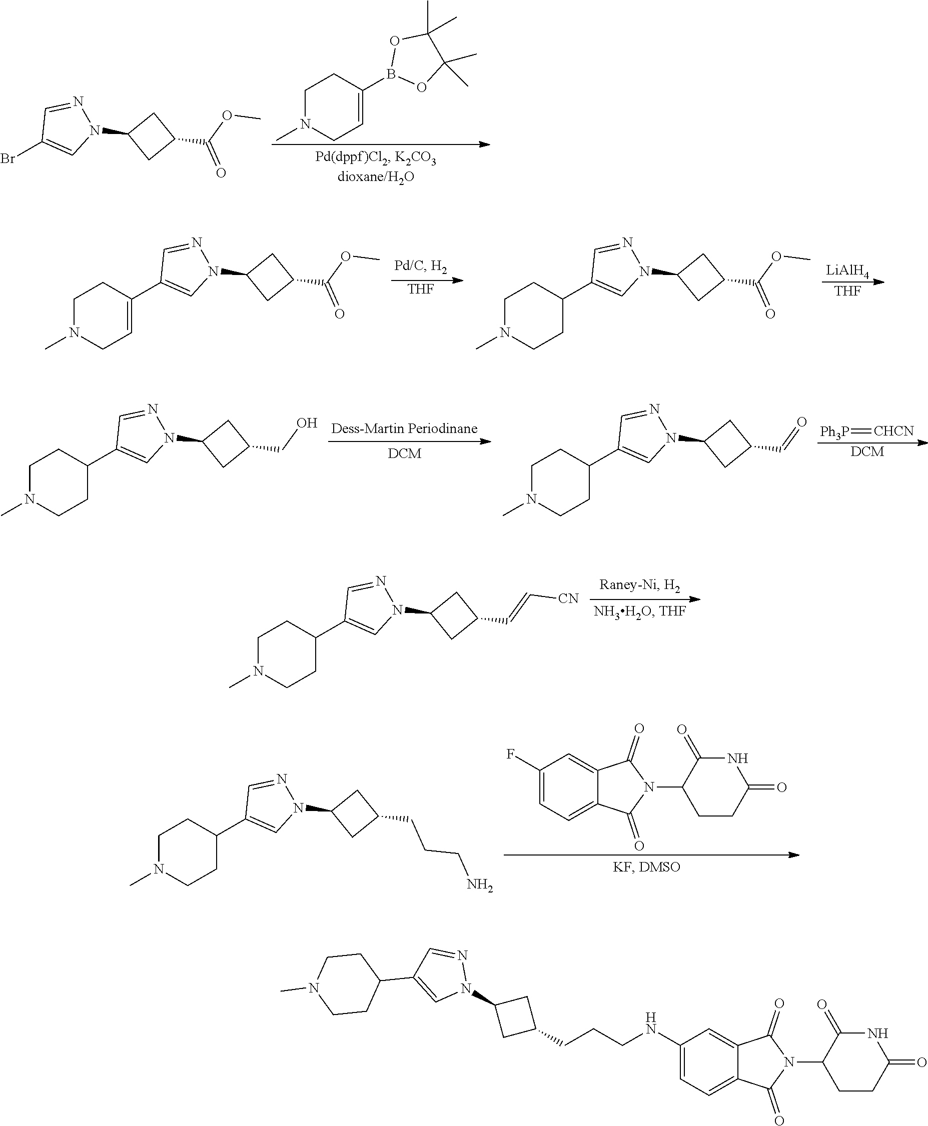

GS-435 was synthesized following the standard procedures for preparing GS-418 and GS-399 (1.2 mg, 1% yield for 7 steps) as a yellow solid. MS (ESI) m/z=533.3 [M+H]⁺.

### Example 394. 2-(2,6-Dioxopiperidin-3-yl)-5-((3-(trans-3-(4-(2-(4-methylpiperazin-1-yl) pyrimidin-4-yl)-1H-pyrazol-1-yl)cyclobutyl)propyl)amino)isoindoline-1,3-dione (GS-436)

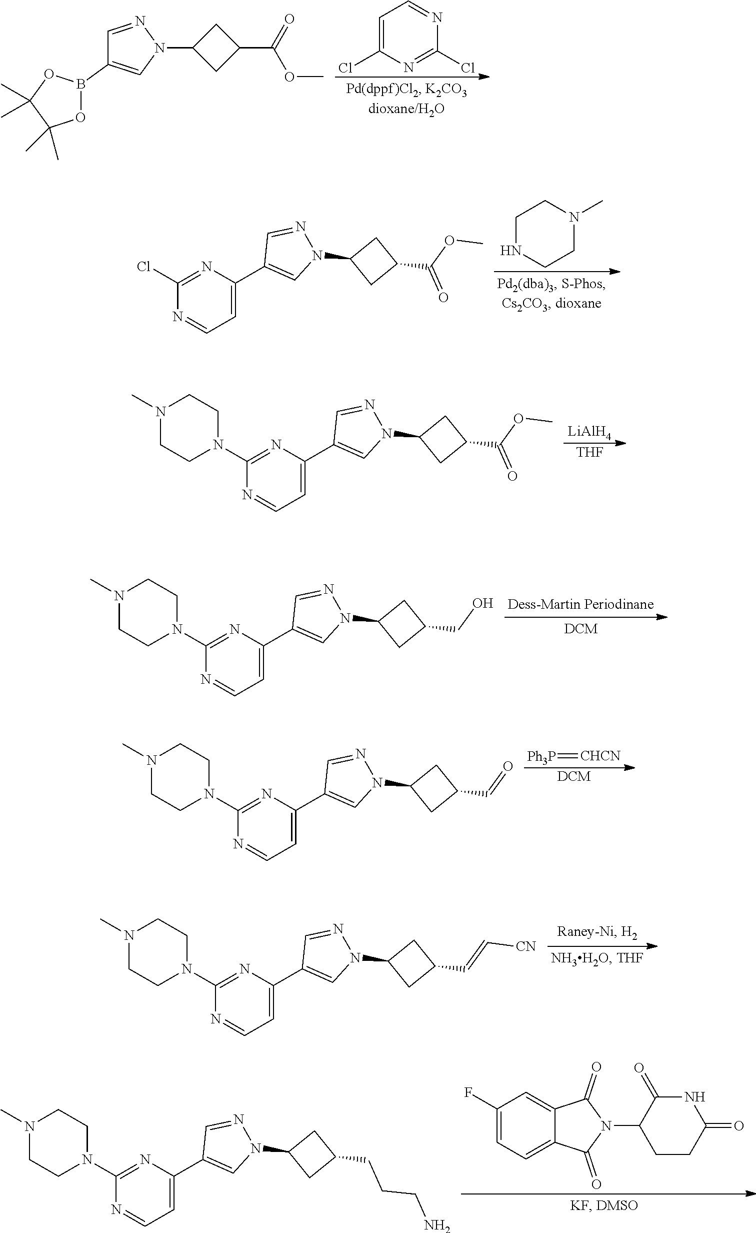

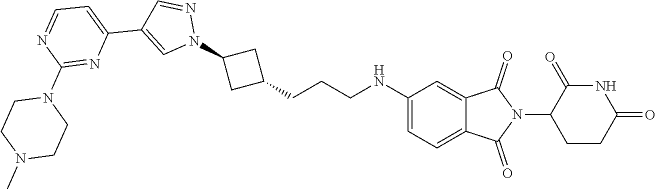 [SMILES: CN1CCN(c2nccc(-c3cnn([C@H]4C[C@H](CCCNc5ccc6c(c5)C(=O)N(C5CCC(=O)NC5=O)C6=O)C4)c3)n2)CC1]

GS-436 was synthesized following the standard procedures for preparing GS-399 (1.1 mg, 1% yield for 7 steps) as a yellow solid. MS (ESI) m/z=612.3 [M+H]⁺.

### Example 395. 2-(2,6-Dioxopiperidin-3-yl)-5-((3-(3-(4-morpholino-1H-pyrazol-1-yl)cyclobutyl)propyl)amino)isoindoline-1,3-dione (GS-437)

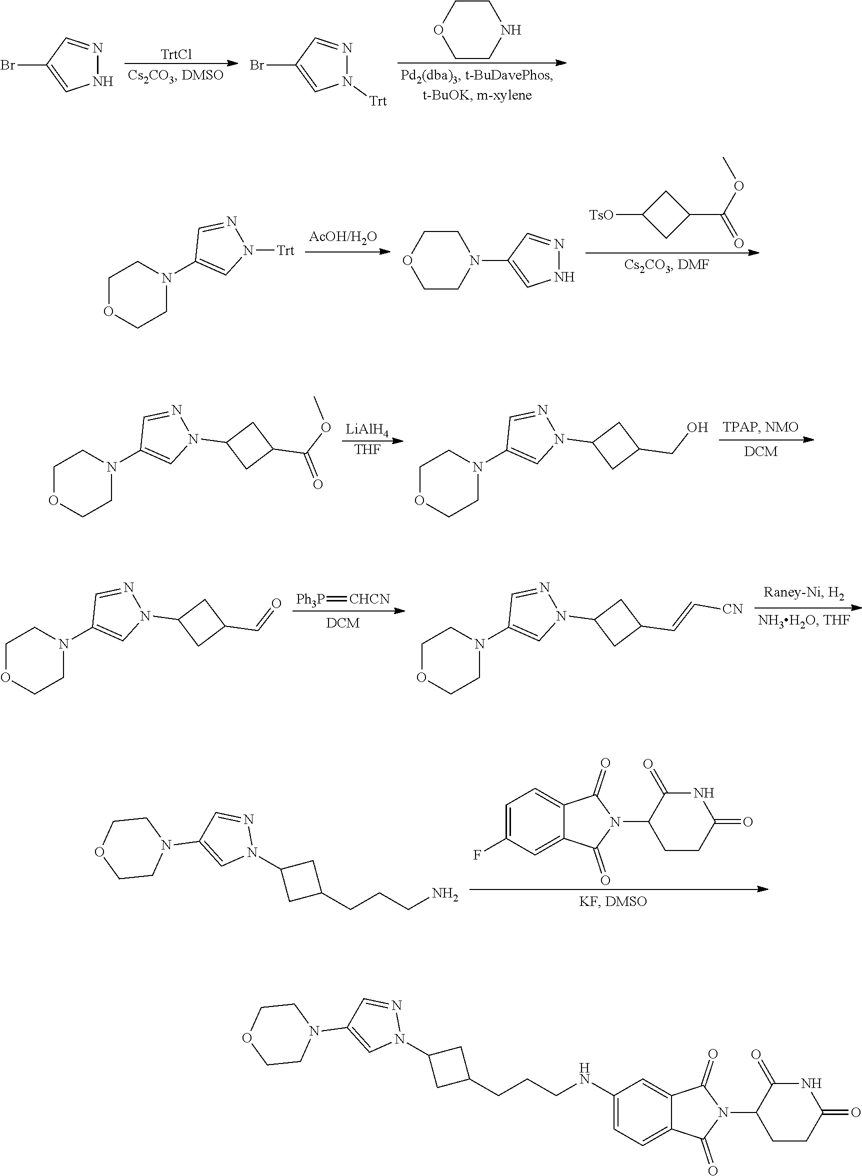

Step 1. Synthesis of 4-bromo-1-trityl-1H-pyrazole

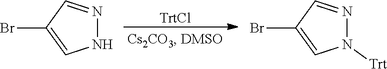

To a solution of 4-bromo-1H-pyrazole (730.0 mg, 5.0 mmol) and (chloromethanetriyl)tribenzene (1668.0 mg, 6.0 mmol) in DMSO (6 mL) was added Cs₂CO₃ (3.25 g, 10.0 mmol) at rt. The reaction mixture was stirred at 90° C. for 2 h. After cooling down to rt, the solution was poured into water (50 mL) and extracted with ethyl acetate (3×50 mL). The combined organic phase was washed with brine (30 mL), dried over anhydrous sodium sulfate, filtered, and concentrated under reduced pressure. The resulting residue was purified by reverse-phase chromatography to provide the desired product (800.0 mg, 41% yield) as a light-yellow solid. MS (ESI) m/z=389.1 [M+H]⁺.

Step 2. Synthesis of 4-(1-trityl-1H-pyrazol-4-yl)morpholine

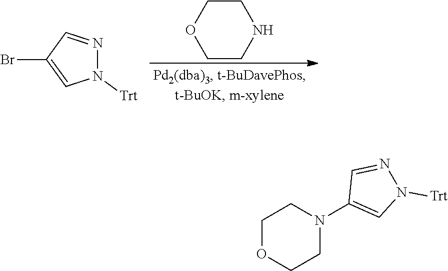

To a suspension of 4-bromo-1-trityl-1H-pyrazole (900.0 mg, 2.31 mmol), morpholine (18.3 mg, 0.10 mmol), t-BuOK (517.4 mg, 4.62 mmol) and t-BuDavephos (78.4 mg, 0.23 mmol) in m-xylene (8.0 mL) was added Pd₂(dba)₃ (46.0 mg, 0.05 mmol) at rt. The reaction mixture was purged with N₂ and stirred at 100° C. for 12 h. After cooling down to rt, the solution was diluted with EtOAc (50 mL) and filtered through Celite. The filtrate was concentrated and purified by reverse-phase chromatography to provide the desired product (520.0 mg, 57% yield) as a yellow solid. MS (ESI) m/z=396.2 [M+H]⁺.

Step 3. Synthesis of 4-(1H-pyrazol-4-yl)morpholine

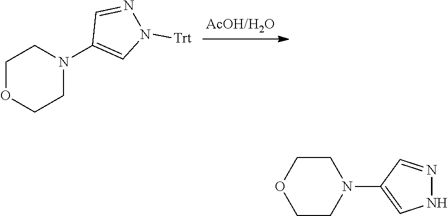

A solution of 4-(1-trityl-1H-pyrazol-4-yl)morpholine (520.0 mg, 1.30 mmol) in H₂O (5 mL) and AcOH (5 mL) was stirred at rt for 10 h. The solution was concentrated under reduced pressure and The resulting residue was purified by reverse-phase chromatography to provide the desired product (150 mg, 75% yield). MS (ESI) m/z=154.1 [M+H]⁺.

Step 4. Synthesis of methyl 3-(4-morpholino-1H-pyrazol-1-yl)cyclobutane-1-carboxylate

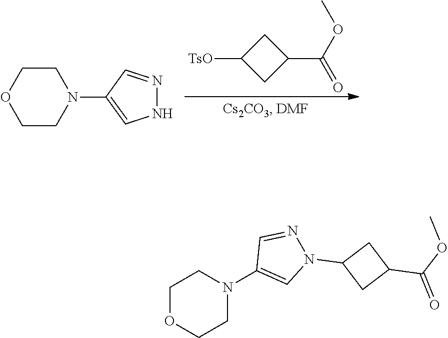 [SMILES: c1n[nH]cc1N1CCOCC1 ; COC(=O)C1CC(OS(=O)(=O)c2ccc(C)cc2)C1 ; COC(=O)C1CC(n2cc(N3CCOCC3)cn2)C1]

To a solution of 4-(1H-pyrazol-4-yl)morpholine (153.0 mg, 1.0 mmol) and methyl 3-(tosyloxy)cyclobutane-1-carboxylate (284.1 mg, 1.0 mmol) in DMF (6 mL) was added Cs₂CO₃ (975 mg, 3.0 mmol) at rt. Then the reaction mixture was stirred at 90° C. for 2 h. After cooling down to rt, the reaction was quenched with H₂O (20 mL) and extracted with ethyl acetate (3×20 mL). The combined organic phase was concentrated and purified by reverse-phase chromatography to provide the desired product (213.6 mg, 80% yield) as a white solid. MS (ESI) m/z=266.1 [M+H]⁺.

Step 5. Synthesis of (3-(4-morpholino-1H-pyrazol-1-yl)cyclobutyl)methanol

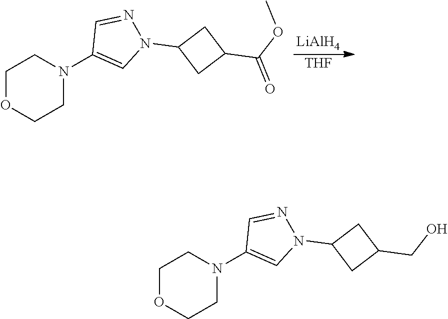

To a solution of methyl 3-(4-morpholino-1H-pyrazol-1-yl)cyclobutane-1-carboxylate (213.6 mg, 0.8 mmol) in THF (6 mL) was added LiAlH₄ (105.6 mg, 2.40 mmol) at 0° C. in portions. The solution was stirred at the same temperature for 1 h, before it was quenched with solid Na₂SO₄ and diluted with EtOAc (15 mL). The suspension was filtered through Celite and the filtrate was concentrated to provide the crude product (150 mg, 79% yield) as a yellow solid which was used in the next step directly. MS (ESI) m/z=238.1 [M+H]⁺.

Step 6. Synthesis of 3-(4-morpholino-2H-pyrrol-2-yl)cyclobutane-1-carbaldehyde

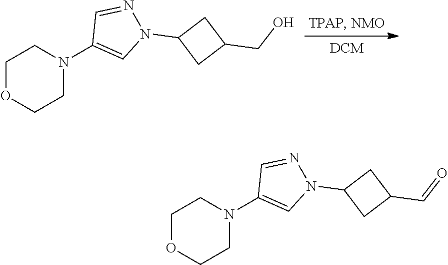 [SMILES: OCC1CC(n2cc(N3CCOCC3)cn2)C1 ; O=CC1CC(n2cc(N3CCOCC3)cn2)C1]

To a solution of (3-(4-morpholino-1H-pyrazol-1-yl)cyclobutyl)methanol (146 mg, 0.61 mmol) in DCM (10 mL) were added tetrapropylammonium perruthenate (20.5 mg, 0.06 mmol), N-methylmorpholine —N-oxide (104.6 mg, 0.92 mmol) at 0° C. The reaction mixture was stirred at rt for 8 h. The solution was diluted with DCM (10 mL) and filtered through Celite. The filtrate was concentrated to provide the crude product (95.2 mg, 66% yield) as a light-yellow solid which was used in the next step directly. MS (ESI) m/z=236.1 [M+H]⁺.

The remaining steps were performed according to the procedures for preparing GS-399 to provide the desired product (10 mg, 27% yield). MS (ESI) m/z=521.3 [M+H]⁺.

### Example 396. 5-(((trans-3-(3-Cyclopropyl-4-morpholino-1H-pyrazol-1-yl)cyclobutyl)methyl)amino)-2-(2,6-dioxopiperidin-3-yl)isoindoline-1,3-dione (GS-438)

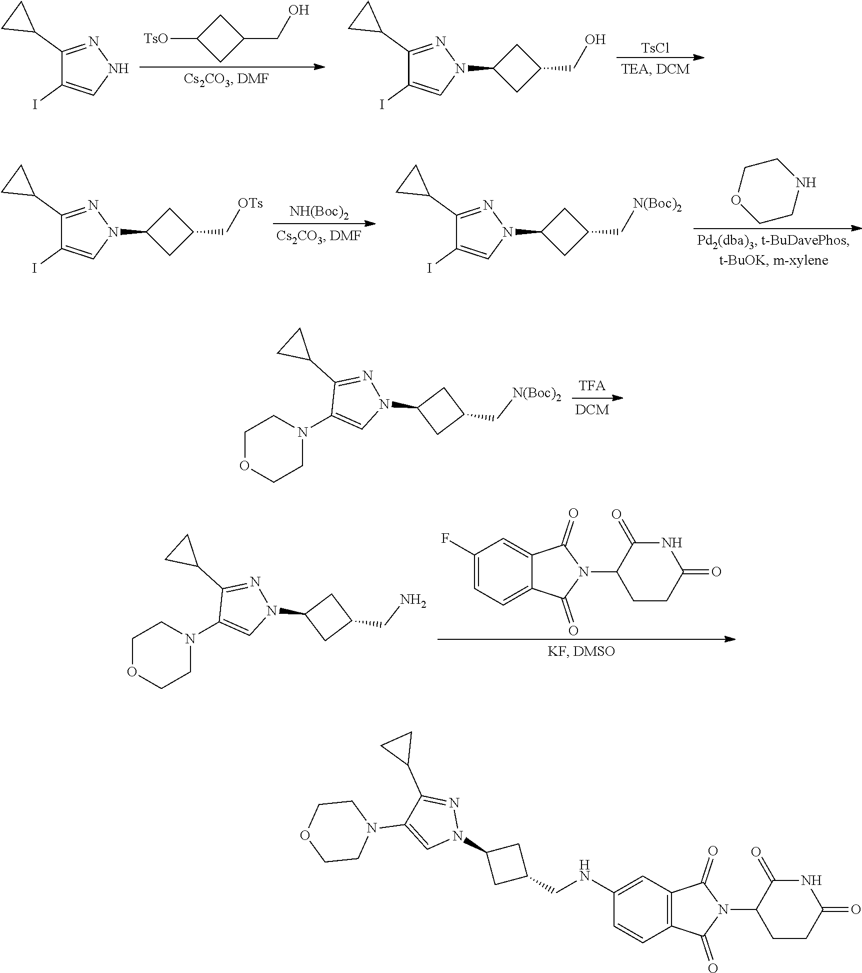

Step 1. Synthesis of (trans-3-(3-cyclopropyl-4-iodo-1H-pyrazol-1-yl)cyclobutyl)methanol

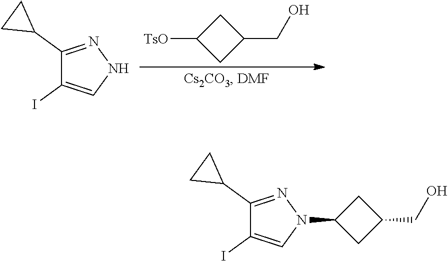 [SMILES: Ic1c[nH]nc1C1CC1 ; Cc1ccc(S(=O)(=O)OC2CC(CO)C2)cc1 ; OC[C@H]1C[C@H](n2cc(I)c(C3CC3)n2)C1]

To a solution of 3-cyclopropyl-4-iodo-1H-pyrazole (468 mg, 2.0 mmol) and cis-3-(hydroxymethyl)cyclobutyl 4-methylbenzenesulfonate (512 mg, 2.0 mmol) in DMF (6 mL) was added Cs₂CO₃ (650 mg, 2.0 mmol) at rt. Then the reaction mixture was stirred at 90° C. for 2 h. After cooling down to rt, the solution was poured into water (50 mL) and extracted with ethyl acetate (3×50 mL). The combined organic phase was washed with brine (30 mL), dried over anhydrous sodium sulfate, filtered, concentrated and purified by reverse-phase chromatography to provide two isomers. The desired trans-isomer (400 mg, 63% yield) was got as a white solid. MS (ESI) m/z=319.0 [M+H]⁺.

Step 2. Synthesis of (trans-3-(3-cyclopropyl-4-iodo-1H-pyrazol-1-yl)cyclobutyl)methyl 4-methylbenzenesulfonate

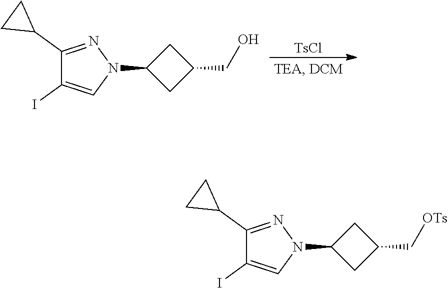 [SMILES: OC[C@H]1C[C@H](n2cc(I)c(C3CC3)n2)C1 ; "TsCl" ; Cc1ccc(S(=O)(=O)OC[C@H]2C[C@H](n3cc(I)c(C4CC4)n3)C2)cc1]

To a solution of (trans-3-(3-cyclopropyl-4-iodo-1H-pyrazol-1-yl)cyclobutyl)methanol (400 mg, 1.25 mmol) in DCM (10 mL) were added TsCl (475 mg, 2.50 mmol) and TEA (505 mg, 5.00 mmol). The reaction mixture was stirred at rt for 4 h, before it was concentrated under vacuum to provide the crude product (500 mg, 85% yield) as a light-yellow solid which was used in the next step directly. MS (ESI) m/z=473.1 [M+H]⁺.

Step 3. Synthesis of tert-butyl-N-tert-butoxycarbonyl-N-[[trans-3-[3-cyclopropyl-4-iodo-pyrazol-1-yl]cyclobutyl]methyl]carbamate

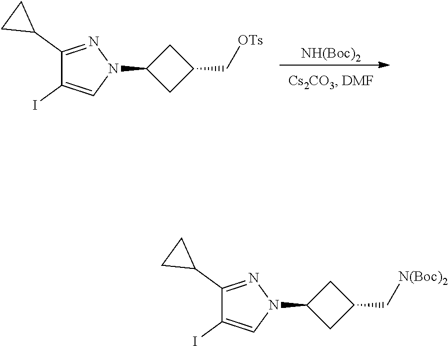

To a solution of (trans-3-(3-cyclopropyl-4-iodo-1H-pyrazol-1-yl)cyclobutyl)methyl 4-methylbenzenesulfonate (500 mg, 1.06 mmol) and tert-butyl N-tert-butoxycarbonylcarbamate (460 mg, 2.12 mmol) in DMF (6 mL) was added Cs₂CO₃ (1.38 g, 4.24 mmol) at rt. The reaction mixture was stirred at 90° C. for 2 h. After cooling down to rt, the solution was poured into water (50 mL) and extracted with ethyl acetate (3×50 mL). The combined organic phase was washed with brine (30 mL), dried over anhydrous sodium sulfate, filtered, and concentrated under reduced pressure. The resulting residue was purified by reverse-phase chromatography to provide the desired product (480 mg, 87% yield) as a light-yellow solid. MS (ESI) m/z=518.2 [M+H]⁺

Step 4. Synthesis of tert-butyl-N-tert-butoxycarbonyl-N-[[trans-3-[(1R)-3-cyclopropyl-4-(1-piperidyl)pyrazol-1-yl]cyclobutyl]methyl]carbamate

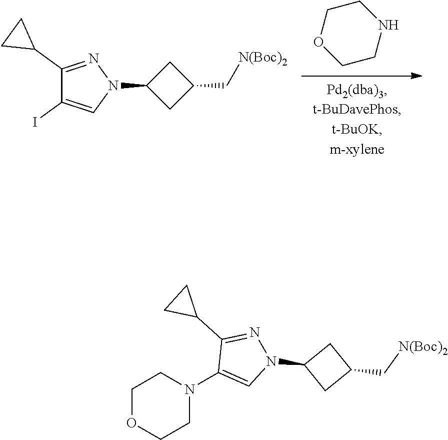

To a mixture of tert-butyl-N-tert-butoxycarbonyl-N-[[trans-3-[3-cyclopropyl-4-iodo-pyrazol-1-yl]cyclobutyl]methyl]carbamate (51.8 mg, 0.10 mmol), morpholine (15.4 mg, 0.10 mmol), t-BuOK (33.6 mg, 0.30 mmol) and t-BuDavephos (34.0 mg, 0.10 mmol) in m-xylene (8 mL) was added Pd₂(dba)₃ (9.1 mg, 0.01 mmol) at rt. The reaction mixture was purged with N₂ and stirred at 100° C. for 12 h. After cooling down to rt, the solution was diluted with EtOAc (20 mL) and filtered through Celite. The filtrate was concentrated and purified by reverse-phase chromatography to provide the desired product (30.9 mg, 65% yield) as a yellow solid. MS (ESI) m/z=477.3 [M+H]⁺.

Step 5. Synthesis of (trans-3-(3-cyclopropyl-4-morpholino-1H-pyrazol-1-yl)cyclobutyl)methanamine

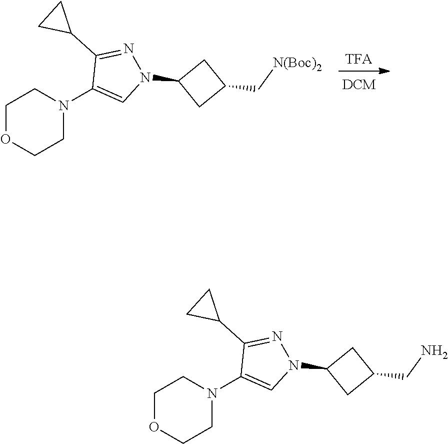

A solution of tert-butyl-N-tert-butoxycarbonyl-N-[[trans-3-[(1R)-3-cyclopropyl-4-(1-piperidyl)pyrazol-1-yl]cyclobutyl]methyl]carbamate (23.8 mg, 0.05 mmol) in DCM (3 mL) and TFA (0.5 mL) was stirred at rt for 1 h. The reaction mixture was concentrated under vacuum to provide the crude product (13.8 mg, 100% yield) as a red brown solid which was used in the next step directly. MS (ESI) m/z=277.2 [M+H]⁺.

Step 6. Synthesis of 5-(((trans-3-(3-cyclopropyl-4-morpholino-1H-pyrazol-1-yl)cyclobutyl)methyl)amino)-2-(2,6-dioxopiperidin-3-yl)isoindoline-1,3-dione

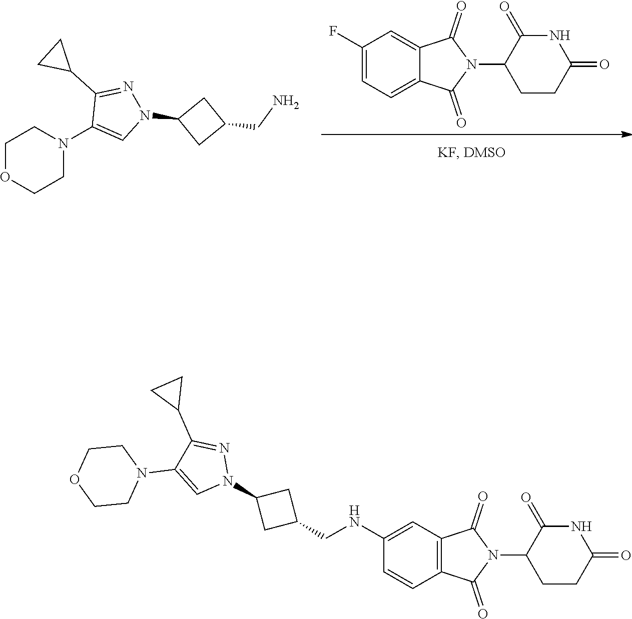

To a mixture of (trans-3-(3-cyclopropyl-4-morpholino-1H-pyrazol-1-yl)cyclobutyl)methanamine (13.8 mg crude, 0.05 mmol) and KF (18.6 mg, 0.32 mmol) in DMSO (0.5 mL) was added 2-(2,6-dioxopiperidin-3-yl)-5-fluoroisoindoline-1,3-dione (27.5 mg, 0.10 mmol) at rt. Then the reaction mixture was stirred at 125° C. under microwave irradiation for 25 min. The solution was cooled to rt and purified by reverse-phase chromatography to provide the desired product (5.1 mg, 19% yield) as a light-yellow solid. MS (ESI) m/z=533.3 [M+H]⁺.

### Example 397. 2-(2,6-Dioxopiperidin-3-yl)-5-(((trans-3-(4-(6-methylpyridin-2-yl)-3-(trifluoromethyl)-1H-pyrazol-1-yl)cyclobutyl)methyl)amino)isoindoline-1,3-dione (GS-439)

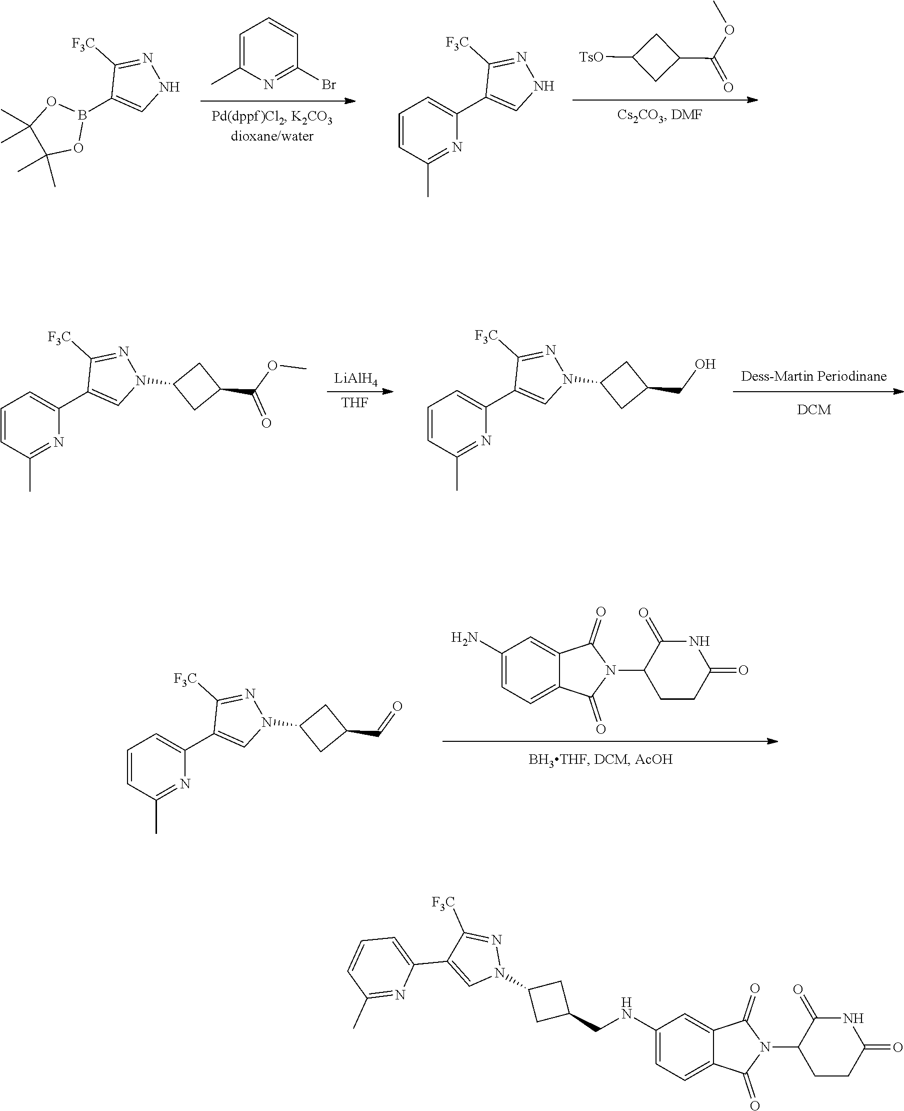

Step 1. Synthesis of 2-methyl-6-(3-(trifluoromethyl)-1H-pyrazol-4-yl)pyridine PGP-15

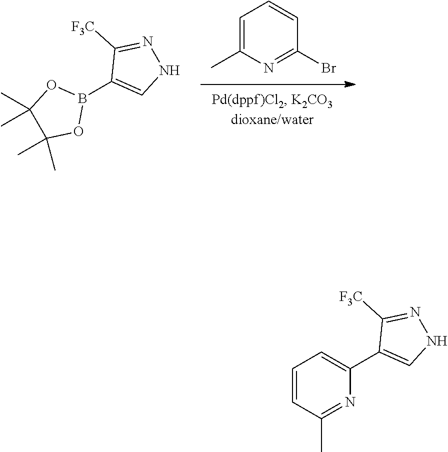

A mixture of 4-(4,4,5,5-tetramethyl-1,3,2-dioxaborolan-2-yl)-3-(trifluoromethyl)-1H-pyrazole (160 mg, 0.61 mmol), 2-bromo-6-methylpyridine (104 mg, 0.62 mmol), Pd(dppf)Cl₂ (15 mg, 0.020 mmol) and K₂CO₃ (161 mg, 1.17 mmol) in dioxane (8 mL) and H₂O (1 mL) was stirred at 100° C. for 1 h under N₂ atmosphere. The reaction was poured into water (15 mL) and extracted with ethyl acetate (3×15 mL). [1727] The combined organic layers were washed with saturated brine (15 mL), dried over anhydrous sodium sulfate, filtered, and concentrated under reduced pressure. The resulting residue was purified by silica gel column chromatography to provide the desired product (103 mg, 75% yield) as a light-yellow solid. MS (ESI) m/z=228.1 [M+H]⁺.

Step 2. Synthesis of methyl 3-(4-(6-methylpyridin-2-yl)-3-(trifluoromethyl)-1H-pyrazol-1-yl)cyclobutane-1-carboxylate

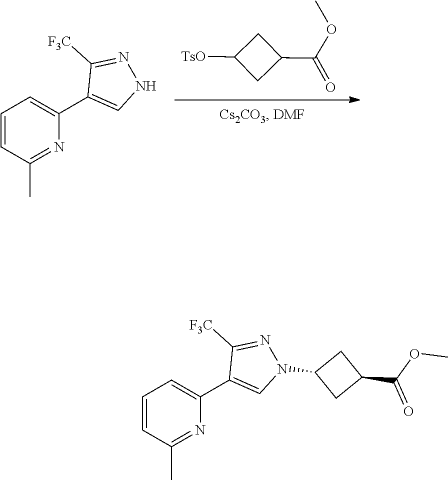

A mixture of 2-methyl-6-(3-(trifluoromethyl)-1H-pyrazol-4-yl)pyridine (100 mg, 0.44 mmol), 2-bromo-6-methylpyridine (138 mg, 0.48 mmol) and Cs₂CO₃ (430 mg, 1.32 mmol) in DMF (3 mL) was stirred at 100° C. for 2 h under N₂ atmosphere. The reaction was poured into water (15 mL) and extracted with ethyl acetate (3×15 mL). The combined organic layers were washed with saturated brine (15 mL), dried over anhydrous sodium sulfate, filtered, and concentrated under reduced pressure. The resulting residue was purified by silica gel column chromatography to provide two isomers. The desired trans-isomer (32 mg, 21% yield) was got as a light-yellow solid. MS (ESI) m/z=340.1 [M+H]⁺.

Step 3. Synthesis of (trans-3-(4-(6-methylpyridin-2-yl)-3-(trifluoromethyl)-1H-pyrazol-1-yl)cyclobutyl)methanol

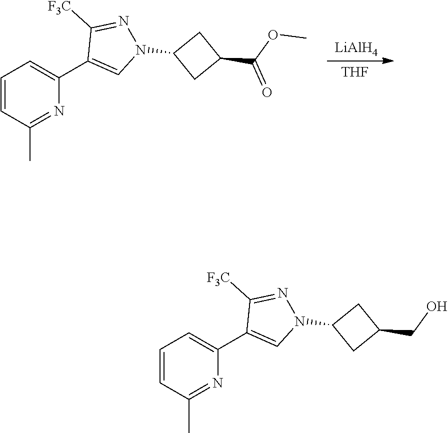 [SMILES: COC(=O)[C@H]1C[C@H](n2cc(-c3cccc(C)n3)c(C(F)(F)F)n2)C1 ; "LiAlH4" ; "THF" ; Cc1cccc(-c2cn([C@H]3C[C@H](CO)C3)nc2C(F)(F)F)n1]

To a solution of methyl 3-(4-(6-methylpyridin-2-yl)-3-(trifluoromethyl)-1H-pyrazol-1-yl)cyclobutane-1-carboxylate (32 mg, 0.09 mmol) in THF (2 mL) was added LiAlH₄ (7 mg, 0.18 mmol) at 0° C. The reaction mixture was stirred at 0° C. for 1 h. The resulting mixture was quenched with Na₂SO₄·10H₂O, filtered, and concentrated under reduced pressure. The resulting residue was purified by silica gel column chromatography to provide the desired product (20 mg, 71% yield) as a light-yellow solid. MS (ESI) m/z=312.1 [M+H]⁺.

Step 4. Synthesis of trans-3-(4-(6-methylpyridin-2-yl)-3-(trifluoromethyl)-1H-pyrazol-1-yl)cyclobutane-1-carbaldehyde

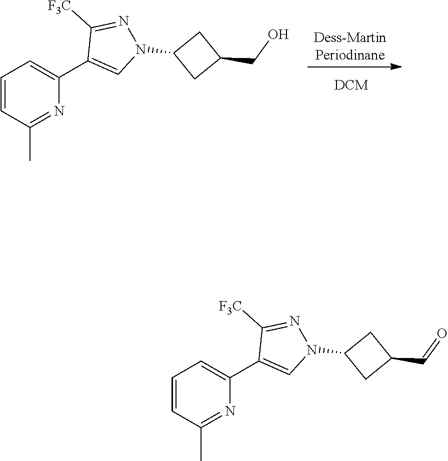

To a solution of (trans-3-(4-(6-methylpyridin-2-yl)-3-(trifluoromethyl)-1H-pyrazol-1-yl)cyclobutyl)methanol (20 mg, 0.06 mmol) in DCM (2 mL) were added NaHCO₃ (10 mg, 0.12 mmol) and Dess-Martin Periodinane (38 mg, 0.09 mmol) at 0° C. The reaction mixture was stirred at 0° C. for 1 h. The mixture was filtered and the filtrate was evaporated under reduced pressure. The resulting residue was purified by silica gel column chromatography to provide the desired product (11 mg, 59% yield) as a light-yellow solid. MS (ESI) m/z=310.1 [M+H]⁺.

Step 5. Synthesis of 2-(2,6-dioxopiperidin-3-yl)-5-(((trans-3-(4-(6-methylpyridin-2-yl)-3-(trifluoromethyl)-1H-pyrazol-1-yl)cyclobutyl)methyl)amino)isoindoline-1,3-dione

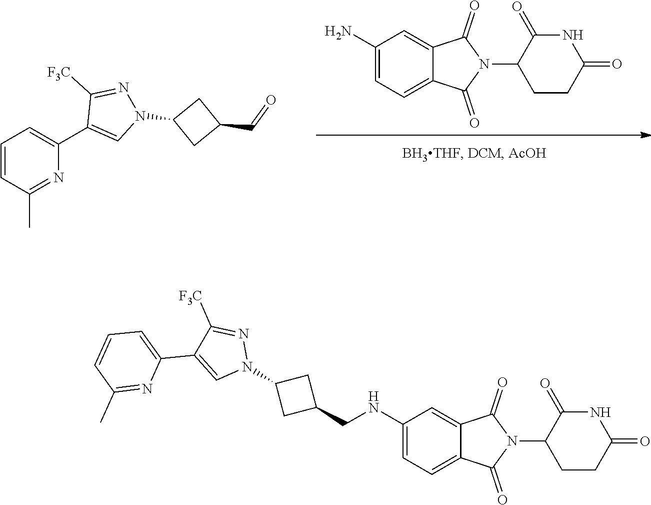

To a mixture of trans-3-(4-(6-methylpyridin-2-yl)-3-(trifluoromethyl)-1H-pyrazol-1-yl)cyclobutane-1-carbaldehyde (11 mg, 0.04 mmol) and 5-amino-2-(2,6-dioxopiperidin-3-yl)isoindoline-1,3-dione (11 mg, 0.04 mmol) in DCM (2 mL) and AcOH (1 mL) was added BH₃·THF (1 M, 0.2 mL) dropwise at rt. The reaction mixture was stirred at rt for 0.5 h, before it was concentrated and purified by prep-TLC to provide the desired product (2.2 mg, 10% yield) as a light-yellow solid. MS (ESI) m/z=567.2 [M+H]⁺.

### Example 398. 5-(((cis-3-(3-Cyclopropyl-4-(6-(4-methylpiperazin-1-yl)pyridin-2-yl)-1H-pyrazol-1-yl)cyclobutyl)methyl)amino)-2-(2,6-dioxopiperidin-3-yl)isoindoline-1,3-dione (GS-440)

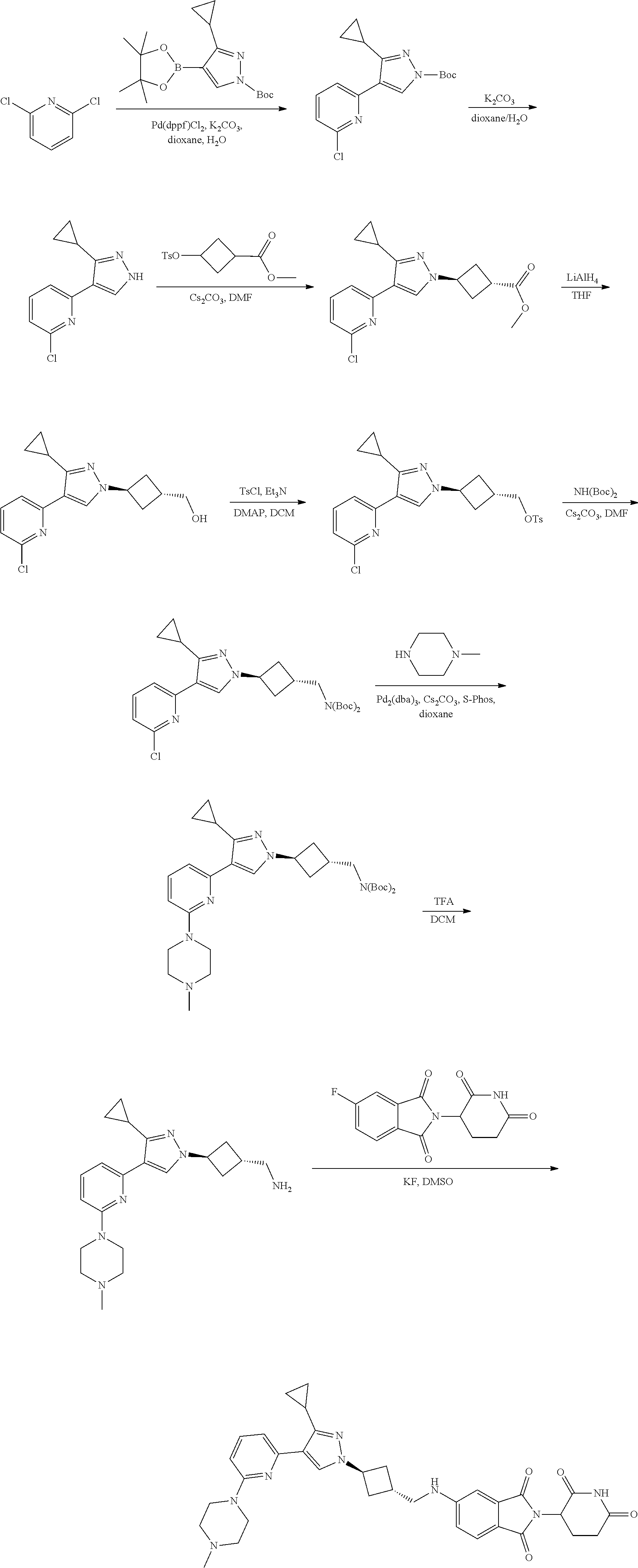

Step 1. Synthesis of tert-butyl 4-(6-chloropyridin-2-yl)-3-cyclopropyl-1H-pyrazole-1-carboxylate

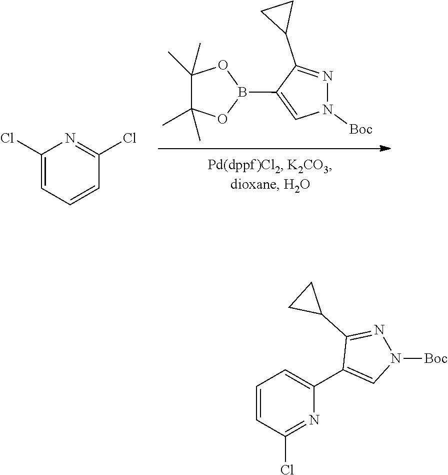

To a solution of 2,6-dichloropyridine (1.4 g, 9.46 mmol) and tert-butyl 3-cyclopropyl-4-(4,4,5,5-tetramethyl-1,3,2-dioxaborolan-2-yl)-1H-pyrazole-1-carboxylate (4.74 g, 14.2 mmol) in THF (25 mL) and H₂O (5 mL) were added K₂CO₃ (3.92 g, 28.3 mmol) and Pd(dppf)C₁₂ (880 mg, 0.9 mmol) at rt under N₂. After stirring at 60° C. for 6 h, the solution was poured into water (50 mL) and extracted with EtOAc (3×30 mL). The combined organic layers were washed with saturated brine, dried over anhydrous sodium sulfate, filtered, and concentrated under reduced pressure. The resulting residue was purified by silica gel flash chromatography to provide the desired product (1.1 g, 36% yield) as a white solid. MS (ESI) m/z=320.2 [M+H]⁺.

Step 2. Synthesis of 2-chloro-6-(3-cyclopropyl-1H-pyrazol-4-yl)pyridine

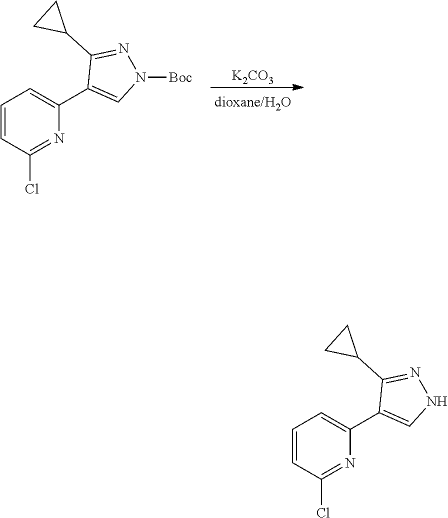

To a mixture of tert-butyl 4-(6-chloropyridin-2-yl)-3-cyclopropyl-1H-pyrazole-1-carboxylate (1.1 g, 3.44 mmol) in dioxane (10 mL) and H₂O (10 mL) was added K₂CO₃ (1.42 g, 10.32 mmol) at rt under N₂. After stirring at 100° C. for 16 h, the resulting black mixture was cooled and diluted with ethyl acetate (40 mL). The solution was washed with saturated brine (2×20 mL). The combined organic phase was dried over sodium sulfate, filtered, concentrated under reduced pressure. The resulting residue was purified by silica gel flash chromatography to provide the desired product (718 mg, 95% yield) as a white solid. MS (ESI) m/z=220.0 [M+H]⁺.

Step 3. Synthesis of methyl cis-3-(4-(6-chloropyridin-2-yl)-3-cyclopropyl-1H-pyrazol-1-yl)cyclobutane-1-carboxylate

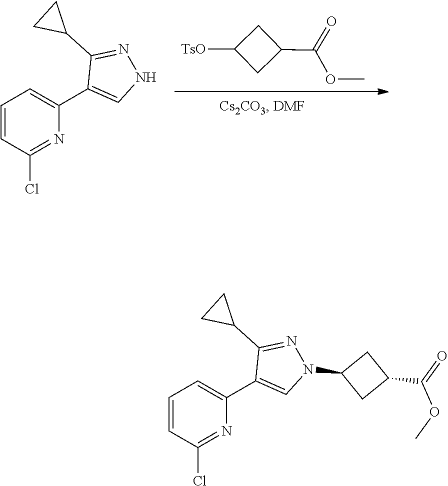

A mixture of 2-chloro-6-(3-cyclopropyl-1H-pyrazol-4-yl)pyridine (718 mg, 3.27 mmol), methyl 3-(p-tolylsulfonyloxy)cyclobutanecarboxylate (1.21 g, 4.3 mmol) and Cs₂CO₃ (3.20 g, 9.8 mmol) in DMF (20 mL) was stirred at 100° C. for 3 h. After cooling down to rt, the reaction mixture was poured into water (100 mL) and extracted with ethyl acetate (3×40 mL). The combined organic layers were washed with saturated brine (50 mL), dried over anhydrous sodium sulfate, filtered, and concentrated under reduced pressure. The resulting residue was purified by silica gel column chromatography to provide two isomers. The desired trans-isomer (230 mg, 21% yield) was got as a light-yellow solid. MS (ESI) m/z=332.2 [M+H]⁺.

Step 4. Synthesis of (cis-3-(4-(6-chloropyridin-2-yl)-3-cyclopropyl-1H-pyrazol-1-yl)cyclobutyl)methanol

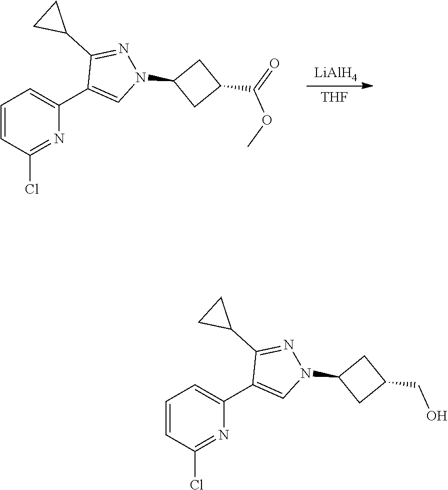 [SMILES: COC(=O)[C@H]1C[C@H](n2cc(-c3cccc(Cl)n3)c(C3CC3)n2)C1 ; "LiAlH4" ; "THF" ; OC[C@H]1C[C@H](n2cc(-c3cccc(Cl)n3)c(C3CC3)n2)C1]

To a solution of methyl cis-3-(4-(6-chloropyridin-2-yl)-3-cyclopropyl-1H-pyrazol-1-yl)cyclobutane-1-carboxylate (230 mg, 0.69 mmol) in THF (20 mL) was added LiAlH₄ (47 mg, 1.39 mmol) at 0° C. under N₂. After the reaction mixture was stirred at rt for 1 h, it was quenched with H₂O (0.1 mL) and anhydrous Na₂SO₄. The precipitate was filtered and the filtrate was concentrated to provide the desired product (205 mg, 97% yield) as a yellow oil. MS (ESI) m/z=304.3 [M+H]⁺.

Step 5. Synthesis of (cis-3-(4-(6-chloropyridin-2-yl)-3-cyclopropyl-1H-pyrazol-1-yl)cyclobutyl)methyl 4-methylbenzenesulfonate

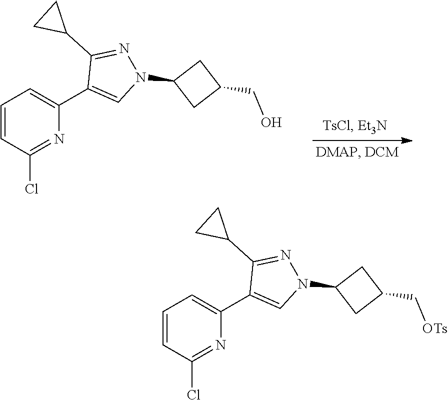 [SMILES: OC[C@H]1C[C@H](n2cc(-c3cccc(Cl)n3)c(C3CC3)n2)C1 ; Cc1ccc(S(=O)(=O)OC[C@H]2C[C@H](n3cc(-c4cccc(Cl)n4)c(C4CC4)n3)C2)cc1]

To a solution of (cis-3-(4-(6-chloropyridin-2-yl)-3-cyclopropyl-1H-pyrazol-1-yl)cyclobutyl)methanol (200 mg, 0.66 mmol) in DCM (10 mL) were added TsCl (251 mg, 1.32 mmol), Et₃N (667 mg, 6.6 mmol) and DMAP (6 mg, 0.06 mmol) at 0° C. After the reaction mixture was stirred at rt for 1 h, it was poured into water (50 mL) and extracted with DCM (3×20 mL). The combined organic layers were washed with saturated brine, dried over anhydrous sodium sulfate, filtered, and concentrated under reduced pressure. The resulting residue was purified by silica gel flash chromatography to provide the desired product (230 mg, 76% yield) as a white solid. MS (ESI) m/z=458.4 [M+H]⁺.

Step 6. Synthesis of cis-tert-butyl N-tert-butoxycarbonyl-N-[[3-[4-(6-chloro-2-pyridyl)-3-cyclopropyl-pyrazol-1-yl]cyclobutyl]methyl]carbamate

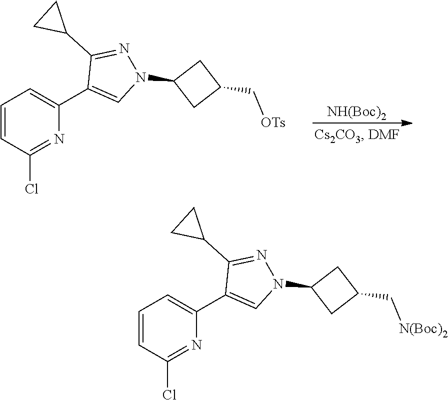

To a solution of (cis-3-(4-(6-chloropyridin-2-yl)-3-cyclopropyl-1H-pyrazol-1-yl)cyclobutyl)methyl 4-methylbenzenesulfonate (230 mg, 0.5 mmol) in DMF (10 mL) were added NH(Boc)₂ (131 mg, 0.6 mmol) and Cs₂CO₃ (491 mg, 1.51 mmol) at rt. Then the reaction was stirred at 100° C. for 2 h. The solution was poured into water (50 mL) and extracted with EtOAc (3×20 mL). The combined organic layers were washed with saturated brine, dried over anhydrous sodium sulfate, filtered, and concentrated under reduced pressure. The resulting residue was purified by silica gel flash chromatography to provide the desired product (240 mg, 95% yield) as a colorless oil. MS (ESI) m/z=503.3 [M+H]⁺.

Step 7. Synthesis of cis-tert-butyl N-tert-butoxycarbonyl-N-[[3-[3-cyclopropyl-4-[6-(4-methylpiperazin-1-yl)-2-pyridyl]pyrazol-1-yl]cyclobutyl]methyl]carbamate

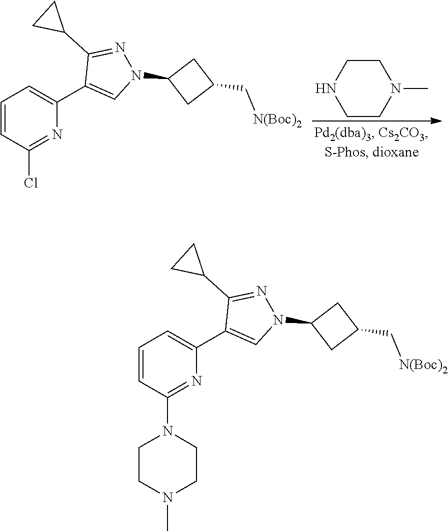

To a mixture of cis-tert-butyl N-tert-butoxycarbonyl-N-[[3-[4-(6-chloro-2-pyridyl)-3-cyclopropyl-pyrazol-1-yl]cyclobutyl]methyl]carbamate (30 mg, 0.06 mmol) in dioxane (5 mL) were added 1-methylpiperazine (12 mg, 0.12 mmol), S-Phos (12 mg, 0.03 mmol), Cs₂CO₃ (58 mg, 0.18 mmol) and Pd₂(dba)₃ (27 mg, 0.03 μmol) at rt under N₂. Then the reaction was stirred at 100° C. for 16 h. The reaction mixture was concentrated and purified by reverse-phase chromatography to provide the desired product (22 mg, 65% yield) as a colorless oil. MS (ESI) m/z=567.4 [M+H]⁺.

Step 8. Synthesis of (cis-3-(3-cyclopropyl-4-(6-(4-methylpiperazin-1-yl)pyridin-2-yl)-1H-pyrazol-1-yl)cyclobutyl)methanamine

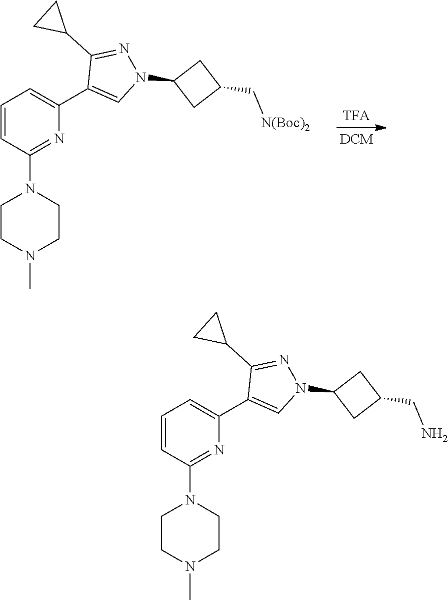

To a solution of cis-tert-butyl N-tert-butoxycarbonyl-N-[[3-[3-cyclopropyl-4-[6-(4-methylpiperazin-1-yl)-2-pyridyl]pyrazol-1-yl]cyclobutyl]methyl]carbamate (22 mg, 0.04 mmol) in DCM (2 mL) was added TFA (1 mL) at 0° C. After stirring at rt for 1 h, the solution was concentrated under vacuum and the resulting residue was used in the next step directly without further purification. MS (ESI) m/z=367.3 [M+H]⁺.

Step 9. Synthesis of 5-(((cis-3-(3-cyclopropyl-4-(6-(4-methylpiperazin-1-yl)pyridin-2-yl)-1H-pyrazol-1-yl)cyclobutyl)methyl)amino)-2-(2,6-dioxopiperidin-3-yl)isoindoline-1,3-dione

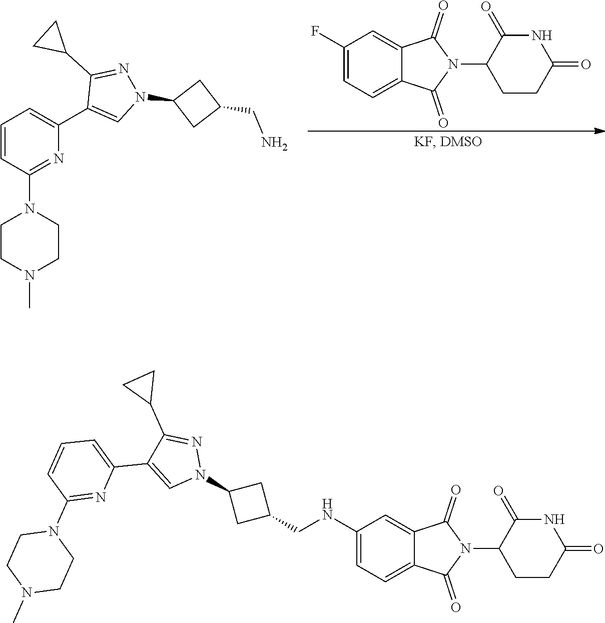 [SMILES: CN1CCN(c2cccc(-c3cn([C@H]4C[C@H](CN)C4)nc3C3CC3)n2)CC1 ; O=C1CCC(N2C(=O)c3ccc(F)cc3C2=O)C(=O)N1 ; CN1CCN(c2cccc(-c3cn([C@H]4C[C@H](CNc5ccc6c(c5)C(=O)N(C5CCC(=O)NC5=O)C6=O)C4)nc3C3CC3)n2)CC1]

To a mixture of (cis-3-(3-cyclopropyl-4-(6-(4-methylpiperazin-1-yl)pyridin-2-yl)-1H-pyrazol-1-yl)cyclobutyl)methanamine (15 mg, 0.04 mmol) in DMSO (1 mL) were added 2-(2,6-dioxo-3-piperidyl)-5-fluoro-isoindoline-1,3-dione (22 mg, 0.08 mmol) and KF (12 mg, 0.2 mmol) at rt. The reaction mixture was stirred at 120° C. under microwave irradiation for 2 h. After cooling down to rt, the reaction was purified by reverse-phase chromatography to provide the desired product (3.65 mg, 12% yield) as a yellow solid. MS (ESI) m/z=623.4 [M+H]⁺.

### Example 399. 5-(((cis-3-(3-Cyclopropyl-4-(6-((tetrahydro-2H-pyran-4-yl)amino)pyridin-2-yl)-1H-pyrazol-1-yl)cyclobutyl)methyl)amino)-2-(2,6-dioxopiperidin-3-yl)isoindoline-1,3-dione (GS-441)

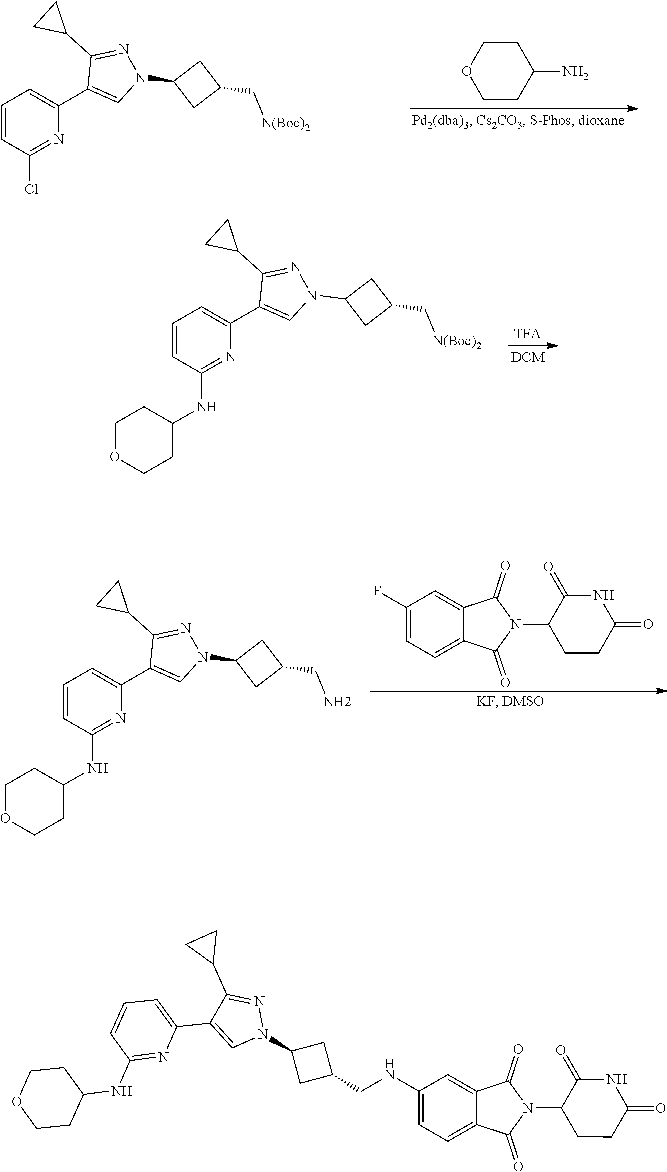

GS-441 was synthesized following the standard procedures for preparing GS-440 (4.7 mg, 1500 yield) as a yellow solid. MS (ESI) m/z=624.4 [M+H]⁺.

### Example 400. 5-(((trans-3-(3-Cyclopropyl-1H-indazol-1-yl)cyclobutyl)methyl)amino)-2-(2,6-dioxopiperidin-3-yl)isoindoline-1,3-dione (GS-442)

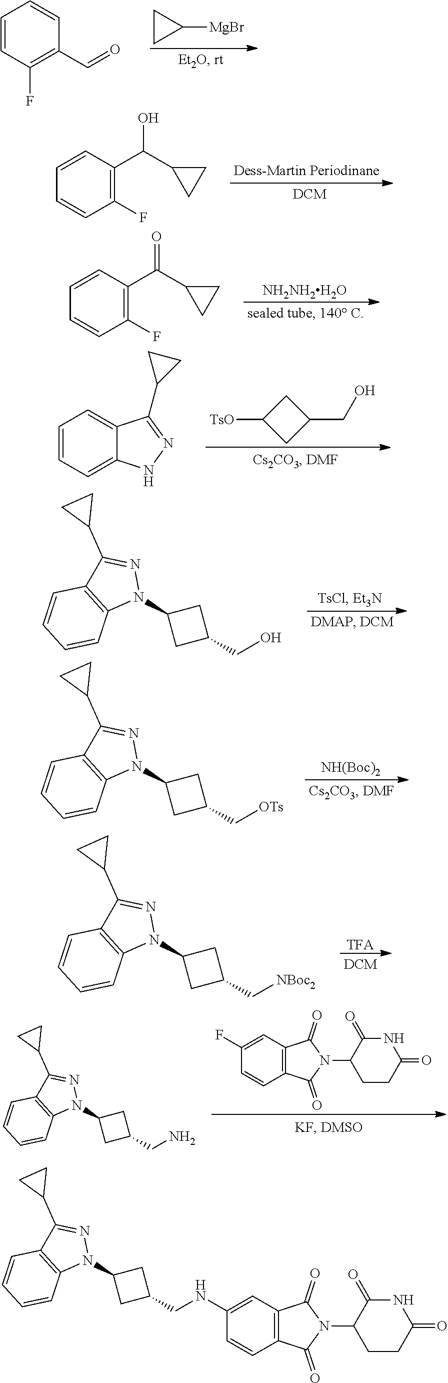

Step 1. Synthesis of cyclopropyl(2-fluorophenyl)methanol

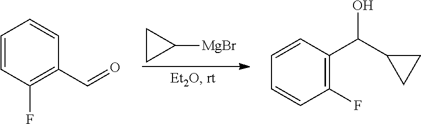

To a solution of 2-fluorobenzaldehyde (20.0 g, 161.3 mmol) in diethyl ether (500 mL) was added a solution of cyclopropylmagnesium bromide (1.0 M, 193.5 mL, 193.5 mmol) in THF dropwise at 0° C. The resulting clear yellow solution was stirred at rt until the reaction was completed by TLC. The reaction was quenched with saturated solution of ammonium chloride and further diluted with water. The resulting mixture was stirred at rt until both layers were clear. The lower aqueous layer was separated and extracted with diethyl ether. The combined organic layers were shaken over magnesium sulfate, filtered, and concentrated under reduced pressure to provide cyclopropyl(2-fluorophenyl)methanol (22 g, crude) as a colorless oil which was used without further purification.

Step 2. Synthesis of cyclopropyl(2-fluorophenyl)methanone

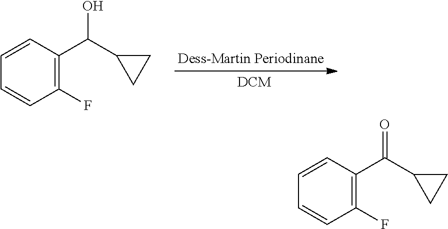 [SMILES: OC(c1ccccc1F)C1CC1 ; O=C(c1ccccc1F)C1CC1]

To a solution of cyclopropyl(2-fluorophenyl)methanol (22 g, crude) in dichloromethane (500 mL) was added Dess-Martin Periodinane (82.0 g, 193.5 mmol). After the reaction was stirred at rt overnight, it was quenched with saturated aq. NaHCO₃ (200 mL) and 10% Na₂SO₃ solution (200 mL). After stirring for 15 min, the solution was extracted with dichloromethane (300 mL×4). The organic phase was dried over anhydrous sodium sulfate, filtered, and concentrated under reduced pressure. The resulting residue was purified by column chromatography on silica gel to provide the desired product (9.8 g, 31% yield for 2 steps) as a colorless oil.

Step 3. Synthesis of 3-cyclopropyl-1H-indazole

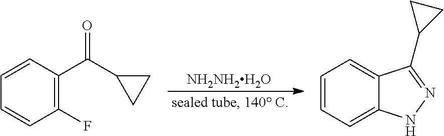

A solution of cyclopropyl(2-fluorophenyl)methanone (14 g, 85.4 mmol) in hydrazine hydrate (50 mL) was heated to 140° C. in sealed tube and kept stirring overnight. After cooling down to rt, the reaction mixture was poured into water and extracted with dichloromethane (100 mL×4). The organic phase was dried over anhydrous sodium sulfate, filtered, and concentrated under reduced pressure. The resulting residue was purified by column chromatography on silica gel to provide the desired product. This product was dissolved in EtOAc (20 mL) and HCl/EA (4 M, 5 mL) was added dropwise. After stirring for 15 min, the resulting precipitate was collected, washed with EtOAc and dried to provide HCl salt of the product (3.8 g, 28% yield) as a white solid. ¹H-NMR (400 MHz, DMSO-D₆) δ:12.51 (s, 1H), 7.75 (d, J=8.0 Hz, 1H), 7.43 (d, J=8.4 Hz, 1H), 7.32-7.28 (m, 1H), 7.07-7.03 (m, 1H), 2.29-2.23 (m, 1H), 1.00-0.91 (m, 4H). MS (ESI) m/z=159.0 [M+H]⁺.

Step 4. Synthesis of methyl (trans-3-(3-cyclopropyl-1H-indazol-1-yl)cyclobutyl)methanol

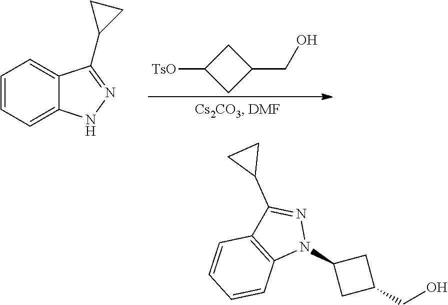 [SMILES: c1ccc2c(C3CC3)n[nH]c2c1 ; Cc1ccc(S(=O)(=O)OC2CC(CO)C2)cc1 ; OC[C@H]1C[C@H](n2nc(C3CC3)c3ccccc32)C1]

To a solution of 3-cyclopropyl-1H-indazole (400 mg, 2.05 mmol) and 3-(hydroxymethyl)cyclobutyl 4-methylbenzenesulfonate (788 mg, 3.08 mmol) in DMF (10 mL) was added Cs₂CO₃ (1.67 g, 5.14 mmol) at rt. The reaction mixture was stirred at 90° C. for 12 h. After cooling down to rt, the reaction was quenched with H₂O (20 mL) and extracted with EtOAc (30 mL×3). The combined organic phase was washed with brine, dried over anhydrous Na₂SO₄, filtered, and concentrated under vacuum. The resulting residue was purified by silica gel column chromatography to provide two isomers. The desired trans-isomer (268 mg, 54% yield) was got as a yellow solid. MS (ESI) m/z=243.2 [M+H]⁺.

Step 5. Synthesis of (trans-3-(3-cyclopropyl-1H-indazol-1-yl)cyclobutyl)methyl 4-methylbenzenesulfonate

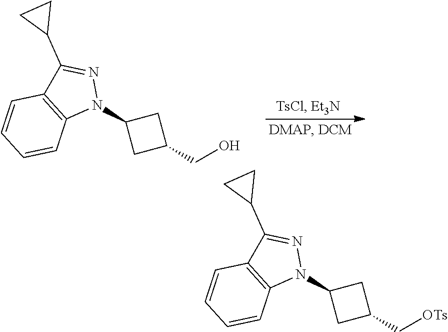

To a solution of (trans-3-(3-cyclopropyl-1H-indazol-1-yl)cyclobutyl)methanol (130 mg, 0.54 mmol), DMAP (65 mg, 0.54 mmol) and TEA (0.25 mL) in DCM (5 mL) was added tosyl chloride (153 mg, 0.80 mmol). The reaction mixture was stirred at 25° C. for 12 h, before it was quenched with H₂O (20 mL) and extracted with DCM (20 mL×3). The combined organic phase was washed with brine and dried over anhydrous Na₂SO₄, filtered, and concentrated under reduced pressure. The resulting residue was purified by silica gel column chromatography to provide the desired product (150 mg, 70% yield) as a yellow solid. MS (ESI) m/z=397.3 [M+H]⁺.

Step 6. Synthesis of tert-butyl ((trans-3-(3-cyclopropyl-1H-indazol-1-yl) cyclobutyl) methyl N-[(tert-butoxy) carbonyl)] carbamate

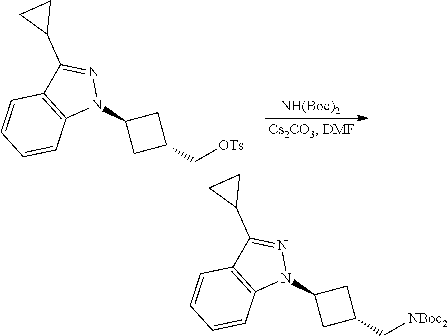

To a solution of (trans-3-(3-cyclopropyl-1H-indazol-1-yl)cyclobutyl)methyl 4-methylbenzenesulfonate (130 mg, 0.33 mmol) and tert-butyl N-tert-butoxycarbonylcarbamate (85 mg, 0.39 mmol) in DMF (4 mL) was added Cs₂CO₃ (213 mg, 0.65 mmol) at rt. The reaction was stirred at 90° C. for 1 h, before it was quenched with H₂O (20 mL) and extracted with EtOAc (20 mL×3). The combined organic phase was washed with brine and dried over anhydrous Na₂SO₄, filtered, and concentrated under reduced pressure. The resulting residue was purified by silica gel chromatography to provide the desired product (150 mg, 94% yield) as a yellow oil. MS (ESI) m/z=442.3 [M+H]⁺.

Step 7. Synthesis of (trans-3-(3-cyclopropyl-1H-indazol-1-yl)cyclobutyl)methanamine

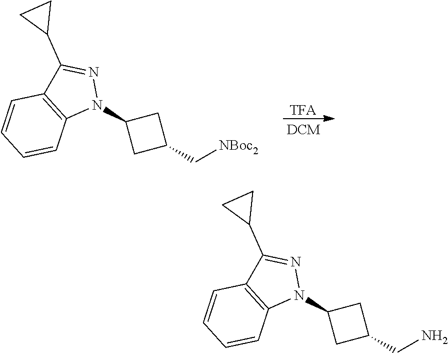

To a solution of tert-butyl ((trans-3-(3-cyclopropyl-1H-indazol-1-yl) cyclobutyl) methyl N-[(tert-butoxy) carbonyl)] carbamate (150 mg, 0.34 mmol) in DCM (5 mL) was added TFA (2 mL) at rt. The mixture was stirred at rt for 1 h, before it was concentrated under vacuum to obtain the crude product (100 mg, 98% yield) as a yellow oil which was used in the next step without further purification. MS (ESI) m/z=242.4 [M+H]⁺.

Step 8. Synthesis of 5-(((trans-3-(3-cyclopropyl-1H-indazol-1-yl)cyclobutyl)methyl)amino)-2-(2,6-dioxopiperidin-3-yl)isoindoline-1,3-dione

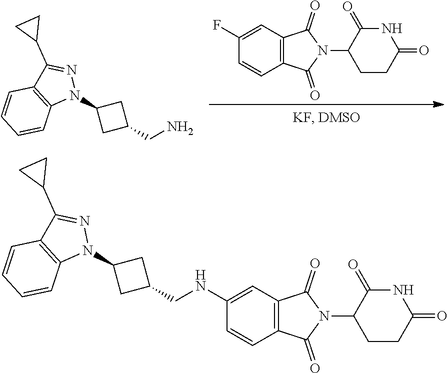

To a solution of (trans-3-(3-cyclopropyl-1H-indazol-1-yl)cyclobutyl)methanamine (50 mg, 0.21 mmol) and 2-(2,6-dioxopiperidin-3-yl)-5-fluoroisoindoline-1,3-dione (68 mg, 0.25 mmol) in DMSO (1 mL) was added DIPEA (0.1 mL). The mixture was stirred at 110° C. for 12 h. After cooling down to rt, the mixture was purified by reverse-phase chromatography to provide the desired product (2.4 mg, 3% yield) as a yellow solid. MS (ESI) m/z=498.3 [M+H]⁺.

### Example 401. 5-((3-(trans-3-(4-(5-Chloropyridin-2-yl)-1H-pyrazol-1-yl)cyclobutyl)propyl)amino)-2-(2,6-dioxopiperidin-3-yl)isoindoline-1,3-dione (GS-443)

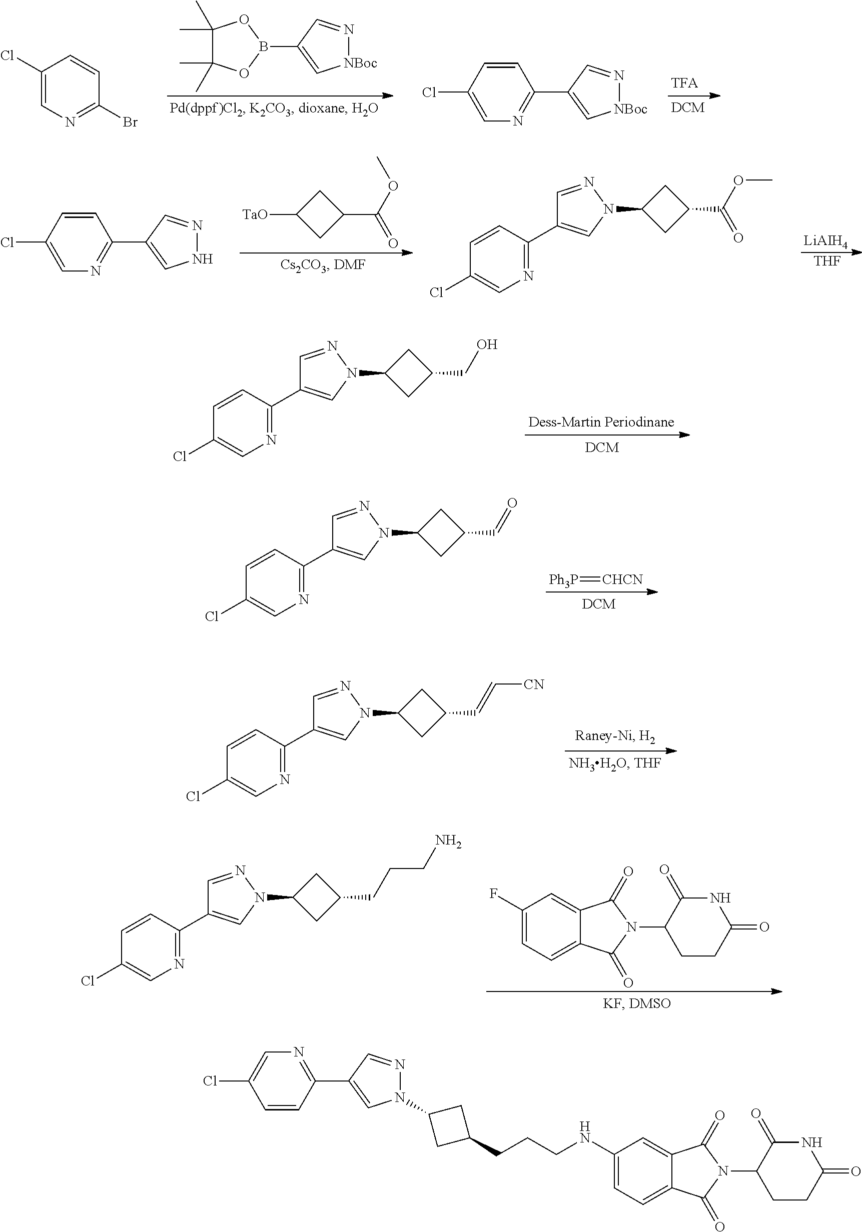

GS-443 was synthesized following the standard procedures for preparing GS-399 (4.8 mg, 26% yield) as a light-yellow solid. MS (ESI) m/z=547.2 [M+H]⁺.

### Example 402. 5-(((trans-3-(3-(difluoromethyl)-4-(6-methylpyridin-2-yl)-1H-pyrazol-1-yl)cyclobutyl)methyl)amino)-2-(2,6-dioxopiperidin-3-yl)isoindoline-1,3-dione (GS-444)

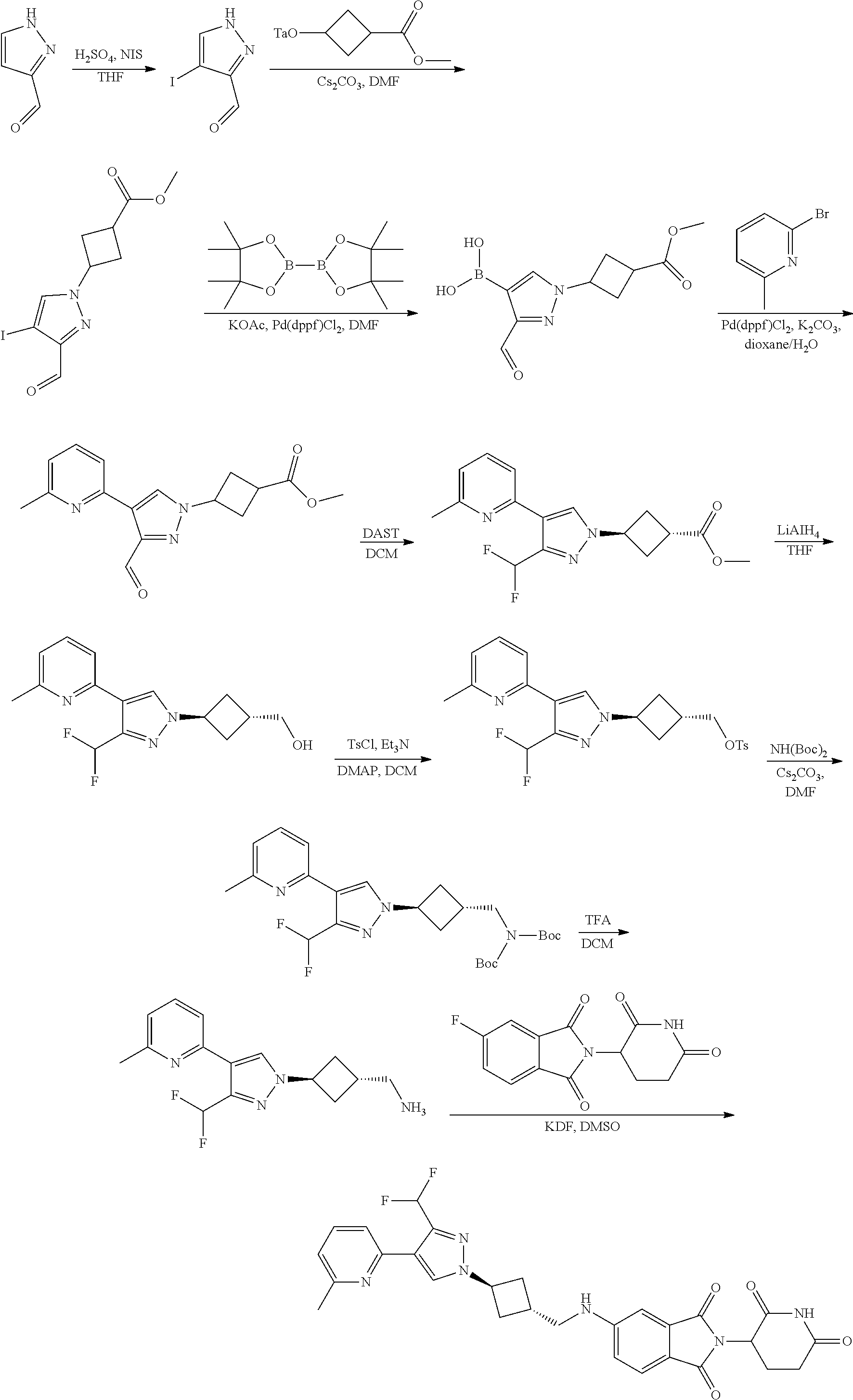

Step 1. Synthesis of 4-iodo-1H-pyrazole-3-carbaldehyde

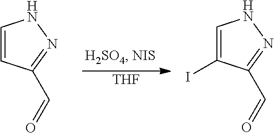 [SMILES: O=Cc1cc[nH]n1 ; O=Cc1n[nH]cc1I ; "THF"]

To a solution of 1H-pyrazole-3-carbaldehyde (3.0 g, 31.25 mmol) and NIS (8.44 g, 37.5 mmol) in THF (150 mL) was added 50% H₂SO₄ (50 mL) dropwise. The mixture was stirred at rt for 4 h. Then water (200 mL) was added and the organic solvent was concentrated. The precipitate was collected and dried to provide the crude product (5.0 g, 72% yield) as a brown solid.

Step 2. Synthesis of methyl 3-(3-formyl-4-iodo-1H-pyrazol-1-yl)cyclobutane-1-carboxylate

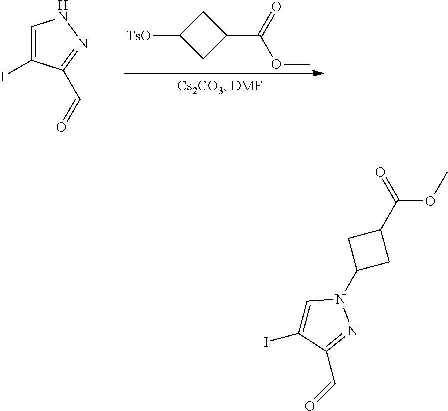 [SMILES: O=Cc1n[nH]cc1I ; COC(=O)C1CC(OS(=O)(=O)c2ccc(C)cc2)C1 ; COC(=O)C1CC(n2cc(I)c(C=O)n2)C1]

To a solution of 4-iodo-1H-pyrazole-3-carbaldehyde (5.0 g, 22.52 mmol) in DMF (50 mL) were added methyl 3-(tosyloxy)cyclobutane-1-carboxylate (7.67 g, 27.03 mmol) and Cs₂CO₃ (14.68 g, 45.04 mmol). The reaction mixture was stirred at 90° C. for 2 h. After cooling down to rt, the solution was poured into water (200 mL) and extracted with ethyl acetate (3×100 mL). The combined organic layers were washed with saturated brine (100 mL), dried over anhydrous sodium sulfate, filtered, and concentrated under reduced pressure. The resulting residue was purified by reverse-phase chromatography to provide the desired product (6.0 g, 79% yield) as a brown solid. MS (ESI) m/z 335.1 [M+H]⁺.

Step 3. Synthesis of (3-formyl-1-(3-(methoxycarbonyl)cyclobutyl)-1H-pyrazol-4-yl)boronic acid

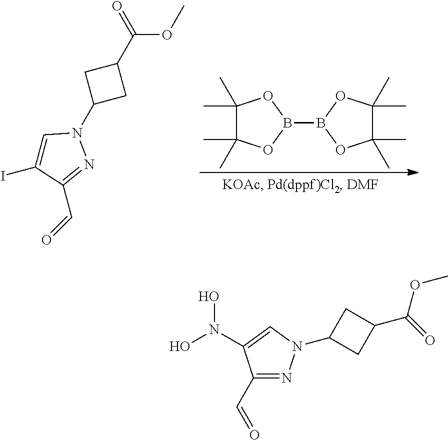

To a solution of methyl 3-(3-formyl-4-iodo-1H-pyrazol-1-yl)cyclobutane-1-carboxylate (3.50 g, 10.48 mmol) in DMF (50 mL) were added 4,4,4′,4′,5,5,5′,5′-octamethyl-2,2′-bi(1,3,2-dioxaborolane) (3.19 g, 12.57 mmol), KOAc (2.05 g, 20.96 mmol) and Pd(dppf)Cl₂ (767 mg, 1.05 mmol) at rt. The reaction mixture was stirred at 90° C. for 2 h under nitrogen. After cooling down to rt, the solution was poured into water (200 mL) and extracted with ethyl acetate (3×100 mL). The combined organic layers were washed with saturated brine (100 mL), dried over anhydrous sodium sulfate, filtered, and concentrated under reduced pressure. The resulting residue was purified by reverse-phase chromatography to provide the desired product (2.0 g, 76% yield) as a yellow oil. MS (ESI) m/z=253.0 [M+H]⁺.

Step 4. Synthesis of methyl 3-(3-formyl-4-(6-methylpyridin-2-yl)-1H-pyrazol-1-yl)cyclobutane-1-carboxylate

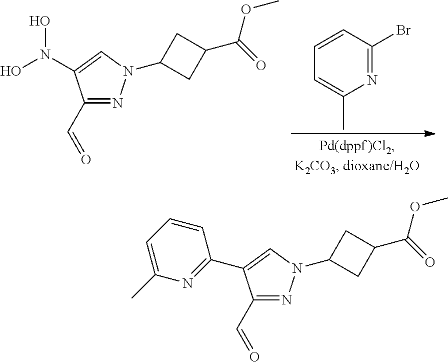

To a solution of (3-formyl-1-(3-(methoxycarbonyl)cyclobutyl)-1H-pyrazol-4-yl)boronic acid (2.0 g, 7.93 mmol) in dioxane (20 mL) and H₂O (5 mL) were added 2-bromo-6-methylpyridine (1.64 g, 9.52 mmol), K₂CO₃ (2.19 g, 15.86 mmol) and Pd(dppf)Cl₂ (577 mg, 0.79 mmol). The reaction mixture was stirred at 80° C. for 2 h under nitrogen. After cooling down to rt, the solution was poured into water (50 mL) and extracted with ethyl acetate (3×30 mL). The combined organic layers were washed with saturated brine (30 mL), dried over anhydrous sodium sulfate, filtered, and concentrated under reduced pressure. The resulting residue was purified by reverse-phase chromatography to provide the desired product (1.1 g, 46% yield) as a yellow solid. MS (ESI) m/z=300.2 [M+H]⁺.

Step 5. Synthesis of methyl trans-3-(3-(difluoromethyl)-4-(6-methylpyridin-2-yl)-1H-pyrazol-1-yl)cyclobutane-1-carboxylate

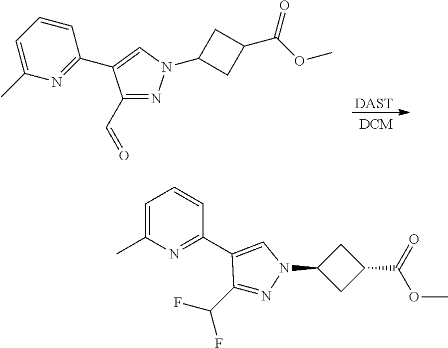

To a solution of methyl 3-(3-formyl-4-(6-methylpyridin-2-yl)-1H-pyrazol-1-yl)cyclobutane-1-carboxylate (1100 mg, 3.68 mmol) in DCM (20 mL) was added DAST (296 mg, 18.39 mmol) dropwise at 0° C. The reaction mixture was warmed to rt and stirred for 0.5 h. The reaction was quenched with water (5 mL). The organic phase was separated, concentrated and purified by reverse-phase chromatography to provide two isomers. The desired trans-isomer (400 mg, 34% yield) was got as a white solid. MS (ESI) m/z=322.3 [M+H]⁺.

The remaining steps were performed according to the procedures for preparing GS-442 to provide the desired product (12 mg, 4% yield over 5 steps) as a yellow solid. MS (ESI) m/z=549.2 [M+H]⁺.

### Example 403. 5-(((trans-3-(3-Cyclopropyl-4-(6-((cis-4-hydroxycyclohexyl)amino)pyridin-2-yl)-1H-pyrazol-1-yl)cyclobutyl)methyl)amino)-2-(2,6-dioxopiperidin-3-yl)isoindoline-1,3-dione (GS-445)

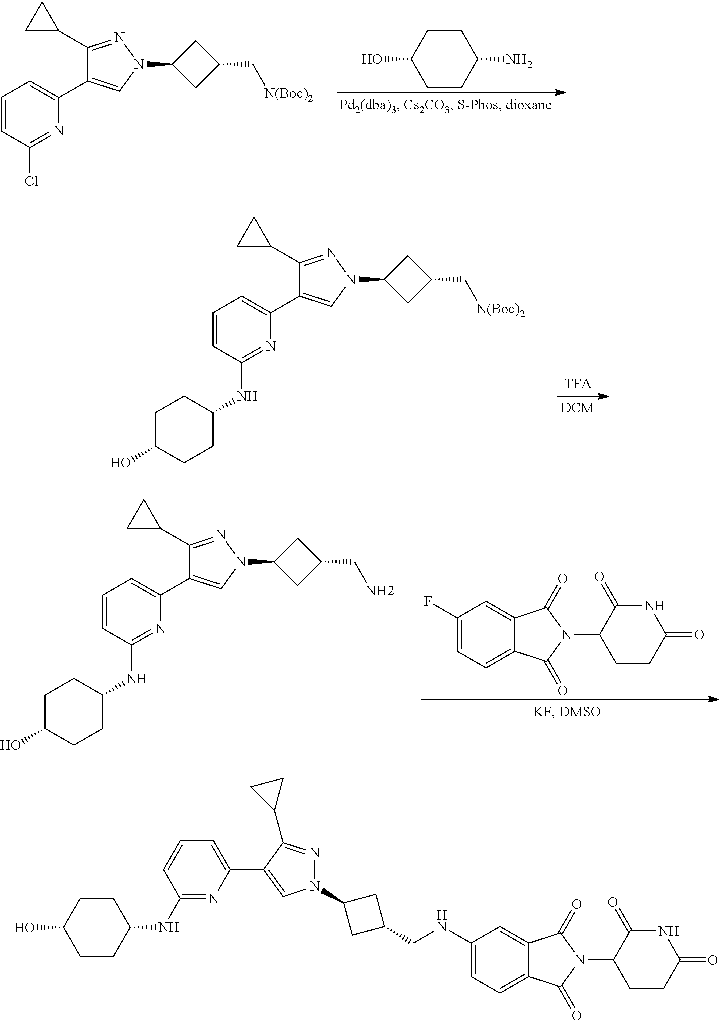

GS-445 was synthesized following the standard procedures for preparing GS-440 (2.5 mg, 22% yield over 3 steps) as a yellow solid. MS (ESI) m/z=624.4 [M+H]⁺.

### Example 404. 2-(2,6-Dioxopiperidin-3-yl)-5-((3-(trans-3-(4-(5-methylpyridin-2-yl)-1H-pyrazol-1-yl)cyclobutyl)propyl)amino)isoindoline-1,3-dione (GS-446)

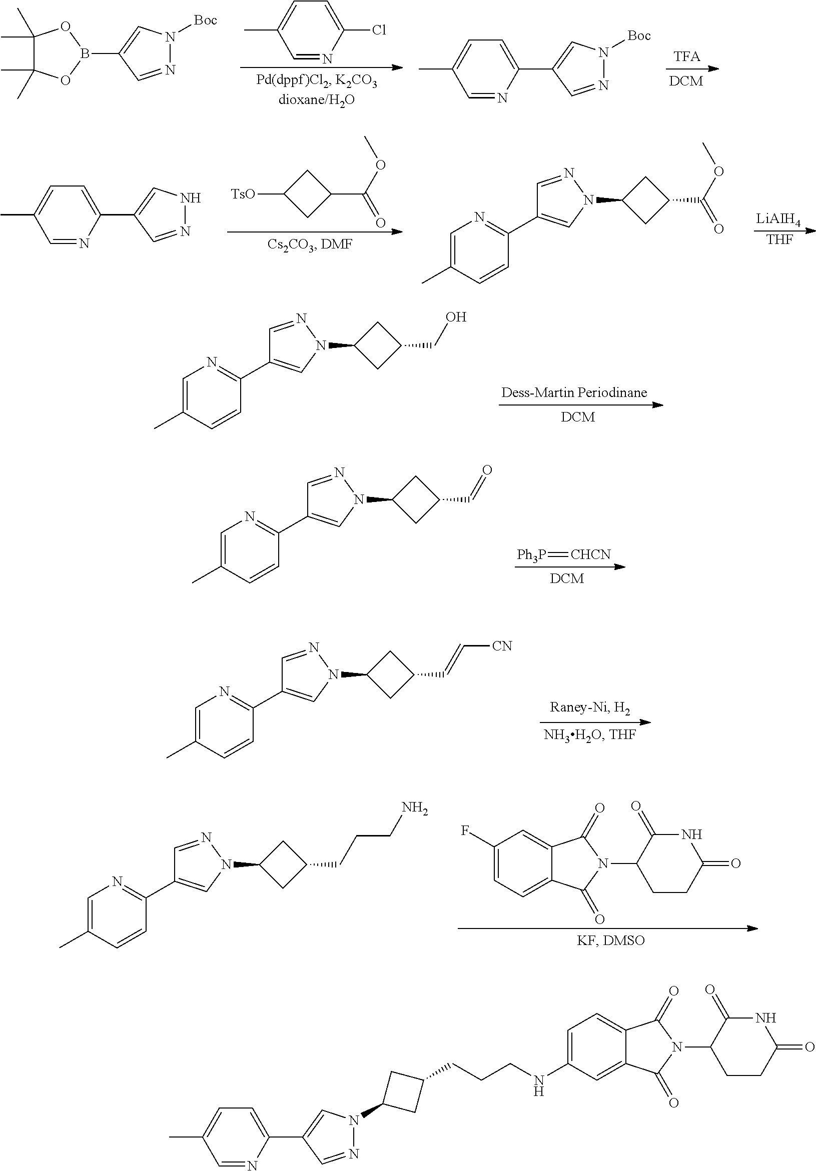

GS-446 was synthesized following the standard procedures for preparing GS-399 (2.1 mg, 1% yield over 8 steps) as a yellow solid. MS (ESI) m/z=527.4 [M+H]⁺.

### Example 405. 5-((3-(trans-3-(4-(6-Cyclopropylpyridin-2-yl)-1H-pyrazol-1-yl)cyclobutyl)propyl)amino)-2-(2,6-dioxopiperidin-3-yl)isoindoline-1,3-dione (GS-447)

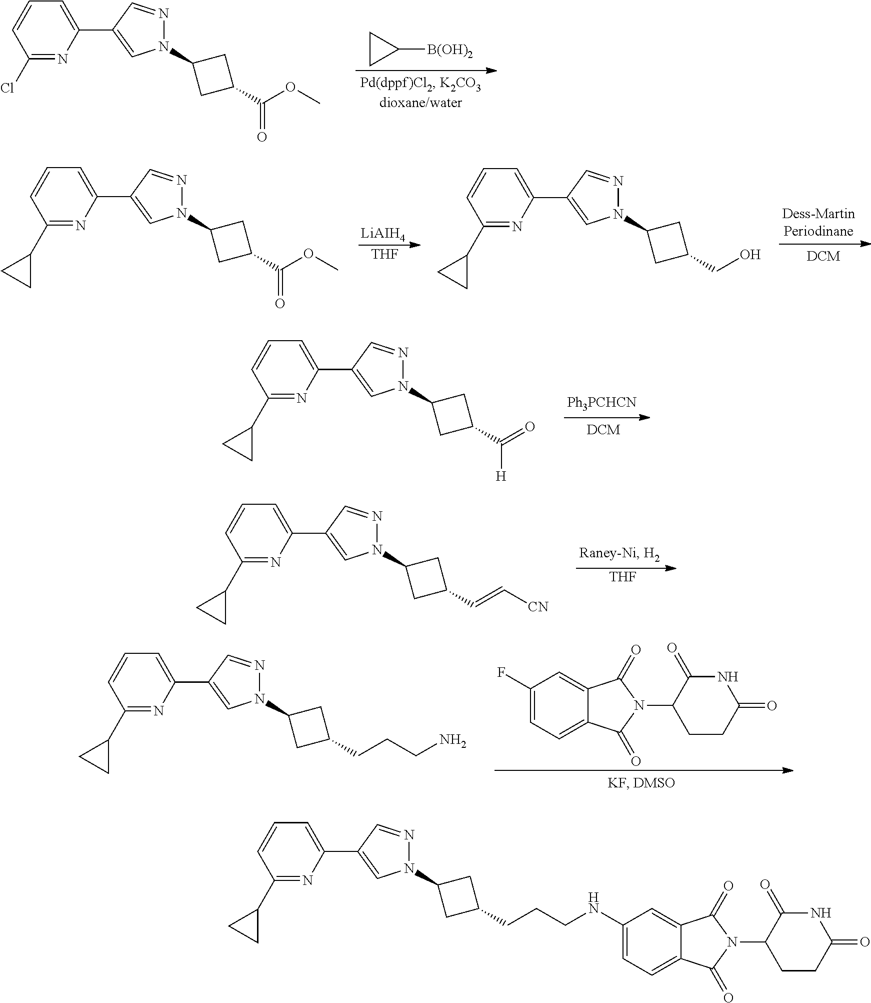

GS-447 was synthesized following the standard procedure for preparing GS-399 (1.1 mg, 32% yield) as a light-yellow solid. MS (ESI) m/z=553.2 [M+H]⁺.

### Example 406. 5-(((trans-3-(3-Cyclopropyl-4-(piperidin-1-yl)-1H-pyrazol-1-yl)cyclobutyl)methyl)amino)-2-(2,6-dioxopiperidin-3-yl)isoindoline-1,3-dione (GS-448)

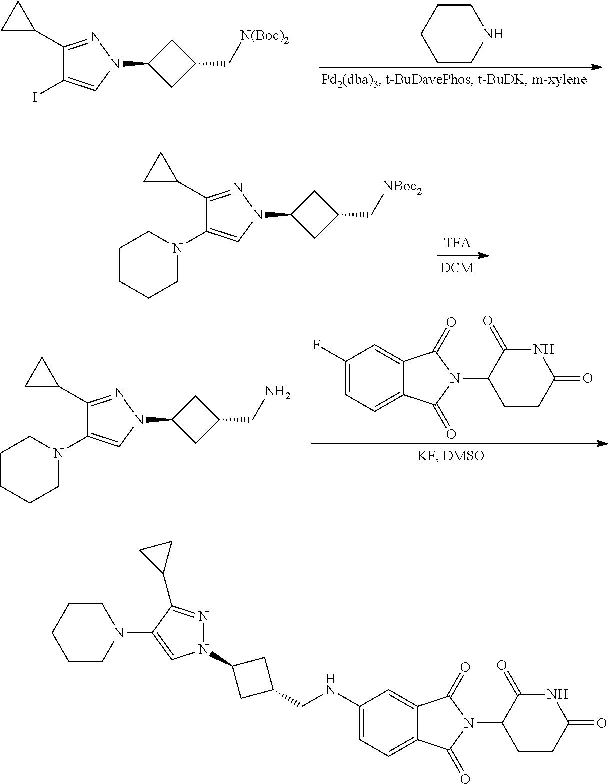

GS-448 was synthesized following the standard procedures for preparing GS-438 (5.9 mg, 24% yield) as a light-yellow solid. MS (ESI) m/z=531.3 [M+H]⁺.

### Example 407. 2-(2,6-Dioxopiperidin-3-yl)-5-(((trans-3-(3-methyl-4-(6-methylpyridin-2-yl)-1H-pyrazol-1-yl)cyclobutyl)methyl)amino)isoindoline-1,3-dione (GS-449)

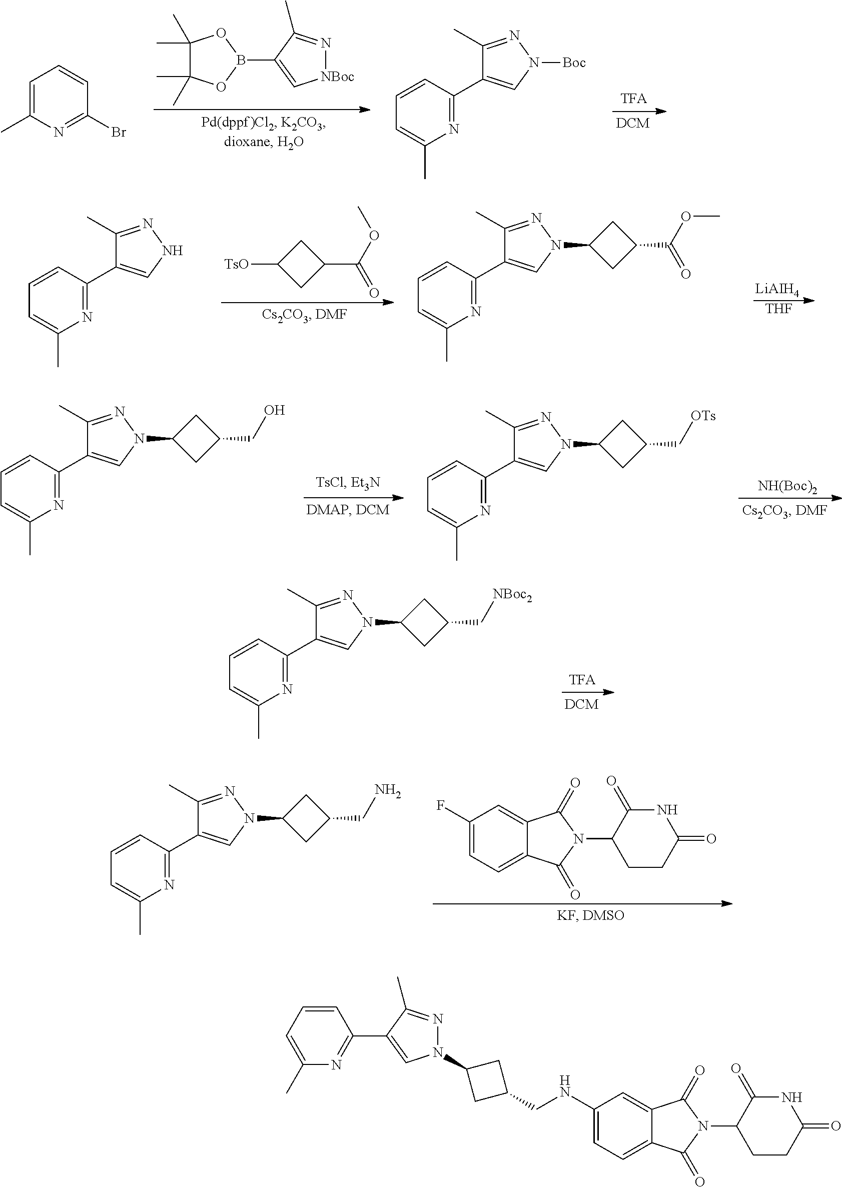

GS-449 was synthesized following the standard procedures for preparing GS-440 (3.4 mg, 1% yield over 8 steps) as a light-yellow solid. MS (ESI) m/z=513.3 [M+H]⁺.

### Example 408. 5-((4-(3-Cyclopropyl-4-(quinoxalin-2-yl)-1H-pyrazol-1-yl)butyl)amino)-2-(2,6-dioxopiperidin-3-yl)isoindoline-1,3-dione (GS-450)

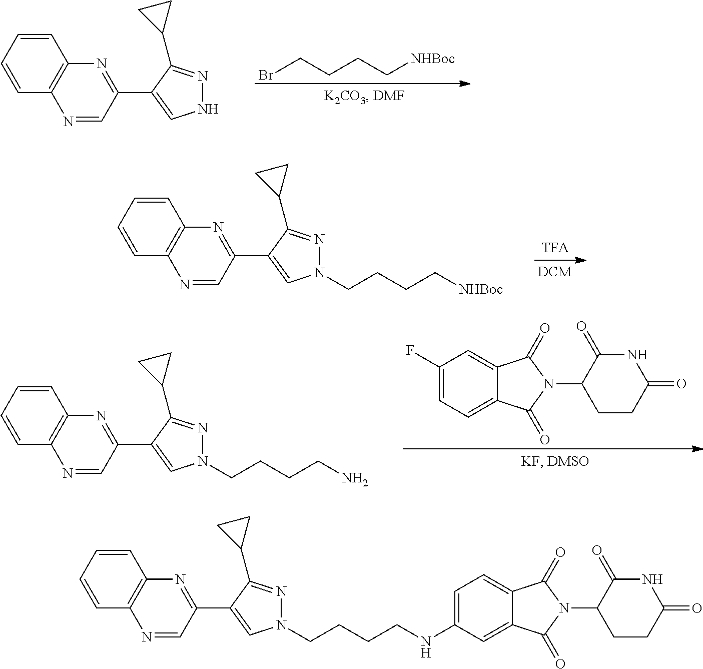

GS-450 was synthesized following the standard procedures for preparing GS-326 (9 mg, 10% yield over 3 steps) as a light-yellow solid. MS (ESI) m/z=564.3 [M+H]⁺.

### Example 409. 5-((3-(trans-3-(4-(6-(Azetidin-1-yl)pyridin-2-yl)-1H-pyrazol-1-yl)cyclobutyl)propyl)amino)-2-(2,6-dioxopiperidin-3-yl)isoindoline-1,3-dione (GS-451)

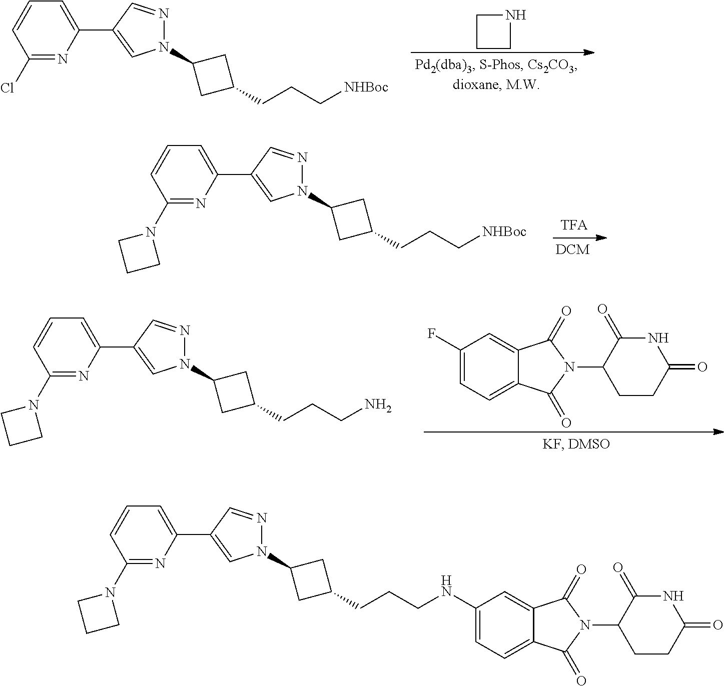

Step 1. Synthesis of tert-butyl (3-(trans-3-(4-(6-(azetidin-1-yl)pyridin-2-yl)-1H-pyrazol-1-yl)cyclobutyl)propyl)carbamate

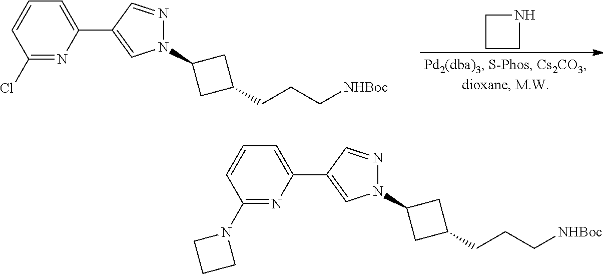

A mixture of the azetidine (8.8 mg, 0.15 mmol), tert-butyl (3-(trans-3-(4-(6-chloropyridin-2-yl)-1H-pyrazol-1-yl)cyclobutyl)propyl)carbamate (30 mg, 0.077 mmol), Pd₂(dba)₃ (7 mg, 0.007 mmol), S-Phos (6.3 mg, 0.015 mmol) and Cs₂CO₃ (50 mg, 0.015 mmol) in dioxane (3 mL) was stirred at 100° C. for 1.5 h under microwave irradiation. After cooling down to rt, the reaction mixture was purified by silica gel column chromatography to provide the desired product (20 mg, 63% yield) as a yellow oil. MS (ESI) m/z=412.3 [M+H]⁺.

Step 2. Synthesis of 3-(trans-3-(4-(6-(azetidin-1-yl)pyridin-2-yl)-1H-pyrazol-1-yl)cyclobutyl)propan-1-amine

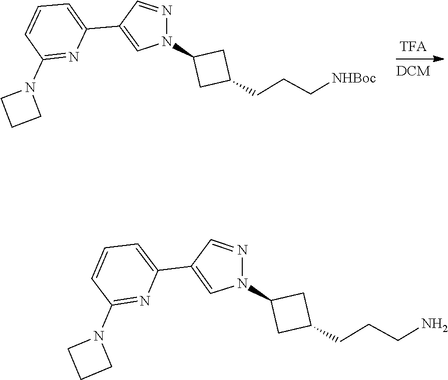 [SMILES: CC(C)(C)OC(=O)NCCC[C@H]1C[C@H](n2cc(-c3cccc(N4CCC4)n3)cn2)C1 ; NCCC[C@H]1C[C@H](n2cc(-c3cccc(N4CCC4)n3)cn2)C1]

A solution of tert-butyl (3-(trans-3-(4-(6-(azetidin-1-yl)pyridin-2-yl)-1H-pyrazol-1-yl)cyclobutyl)propyl)carbamate (30 mg, 72.90 μmol) in DCM (2 mL) and TFA (1 mL) was stirred at it for 1 h. The mixture was concentrated under vacuum to obtain the desired product (17 mg, 77% yield) as a yellow oil. MS (ESI) m/z=312.1 [M+H]⁺.

Step 3. Synthesis of 5-((3-(trans-3-(4-(6-(azetidin-1-yl)pyridin-2-yl)-1H-pyrazol-1-yl)cyclobutyl)propyl)amino)-2-(2,6-dioxopiperidin-3-yl)isoindoline-1,3-dione

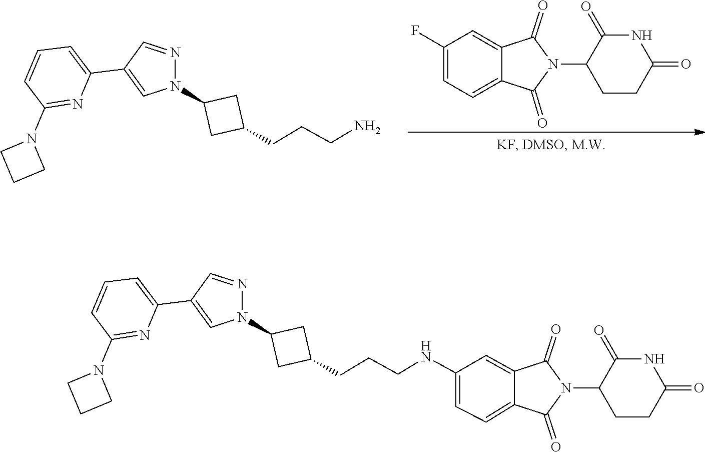 [SMILES: NCCC[C@H]1C[C@H](n2cc(-c3cccc(N4CCC4)n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cccc(N7CCC7)n6)cn5)C4)cc3C2=O)C(=O)N1]

A mixture of 3-(trans-3-(4-(6-(azetidin-1-yl)pyridin-2-yl)-1H-pyrazol-1-yl)cyclobutyl)propan-1-amine (15 mg, 0.048 mmol), 2-(2,6-dioxopiperidin-3-yl)-5-fluoroisoindoline-1,3-dione (13.3 mg, 0.048 mmol) and KF (14 mg, 0.24 mmol) in DMSO (1 mL) was stirred at 130° C. for 0.5 h under microwave irradiation. After cooling down to rt, the mixture was purified by reverse-phase chromatography to provide the desired product (1.6 mg, 6% yield) as a yellow solid. MS (ESI) m/z=568.4 [M+H]⁺.

### Example 410. 5-(((trans-3-(3-Cyclopropyl-4-(4-hydroxypiperidin-1-yl)-1H-pyrazol-1-yl)cyclobutyl)methyl)amino)-2-(2,6-dioxopiperidin-3-yl)isoindoline-1,3-dione (GS-452)

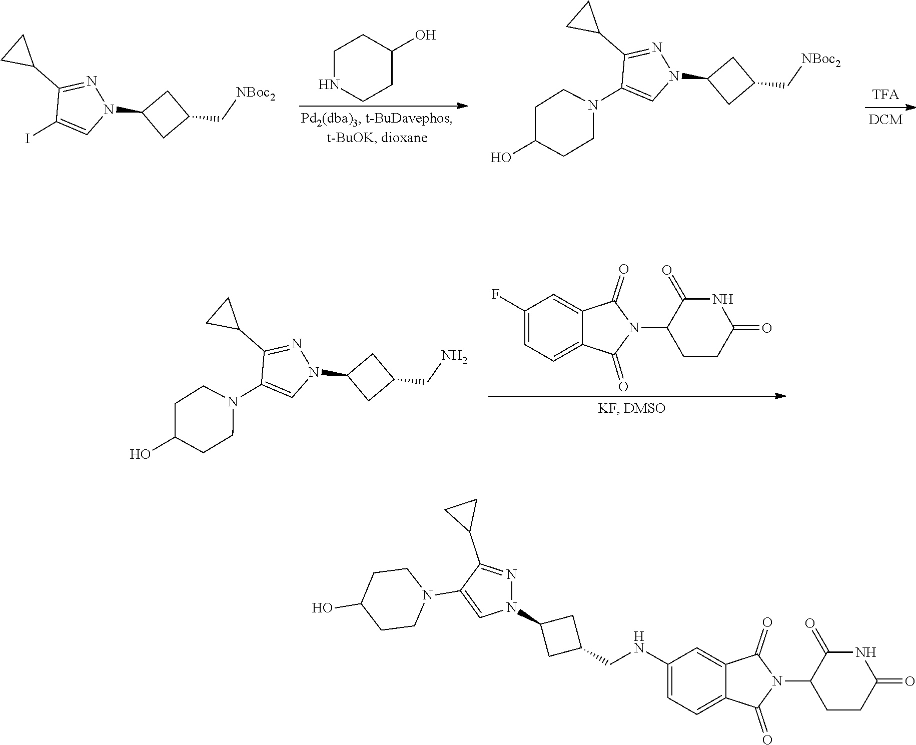

GS-452 was synthesized following the standard procedures for preparing GS-438 (1.5 mg, 11% yield) as a light-yellow solid. MS (ESI) m/z=547.3 [M+H]⁺.

### Example 411. 2-(2,6-Dioxopiperidin-3-yl)-5-((3-(trans-3-(4-(6-isopropylpyridin-2-yl)-1H-pyrazol-1-yl)cyclobutyl)propyl)amino)isoindoline-1,3-dione (GS-453)

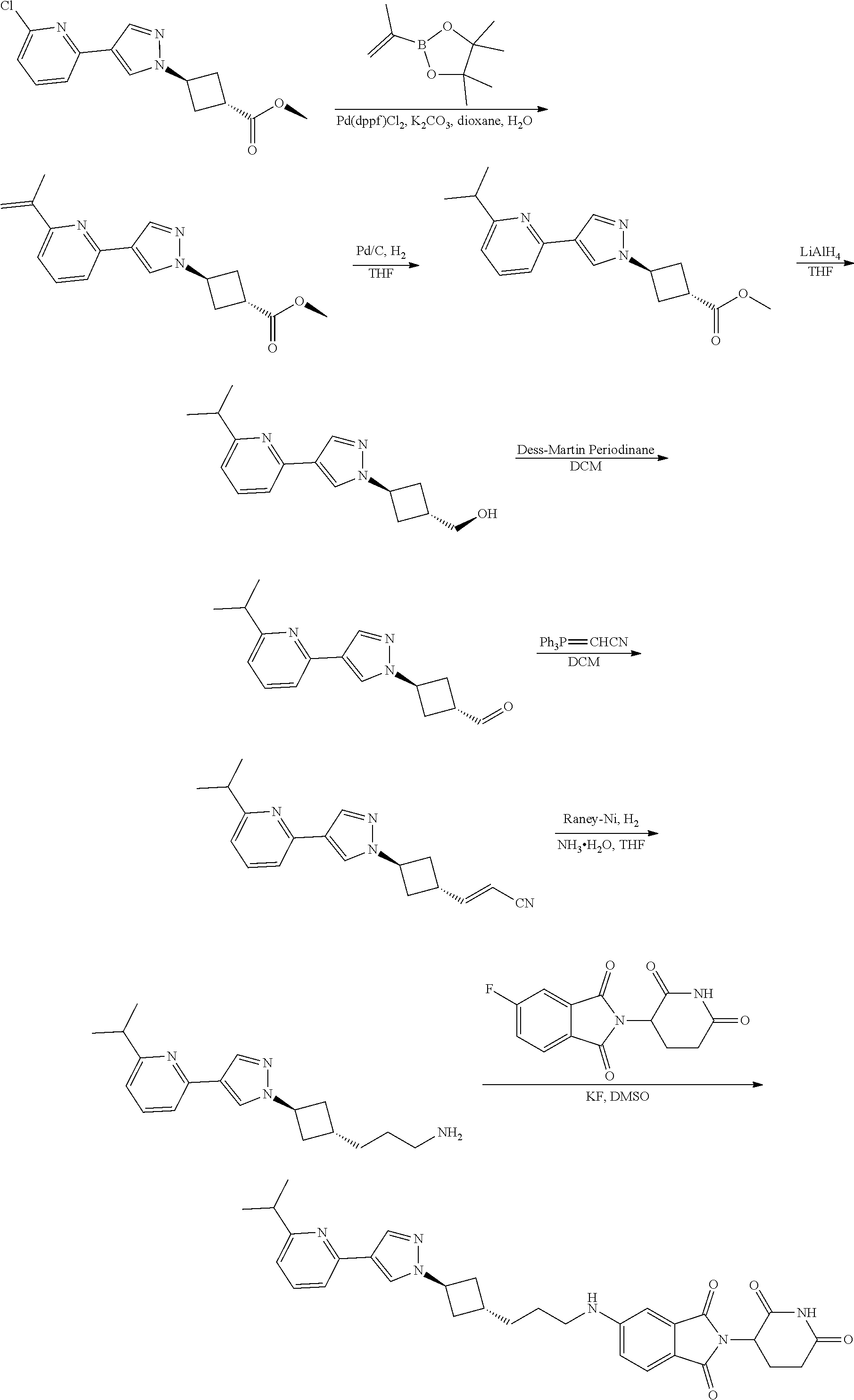

GS-453 was synthesized following the procedures for preparing GS-418 and GS-399 (2 mg, 11% yield) as a yellow solid. MS (ESI) m/z=555.3 [M+H]⁺.

### Example 412. 5-(((trans-3-(3-Cyclopropyl-4-(6-((1-methylpiperidin-4-yl)amino)pyridin-2-yl)-1H-pyrazol-1-yl)cyclobutyl)methyl)amino)-2-(2,6-dioxopiperidin-3-yl)isoindoline-1,3-dione (GS-454)

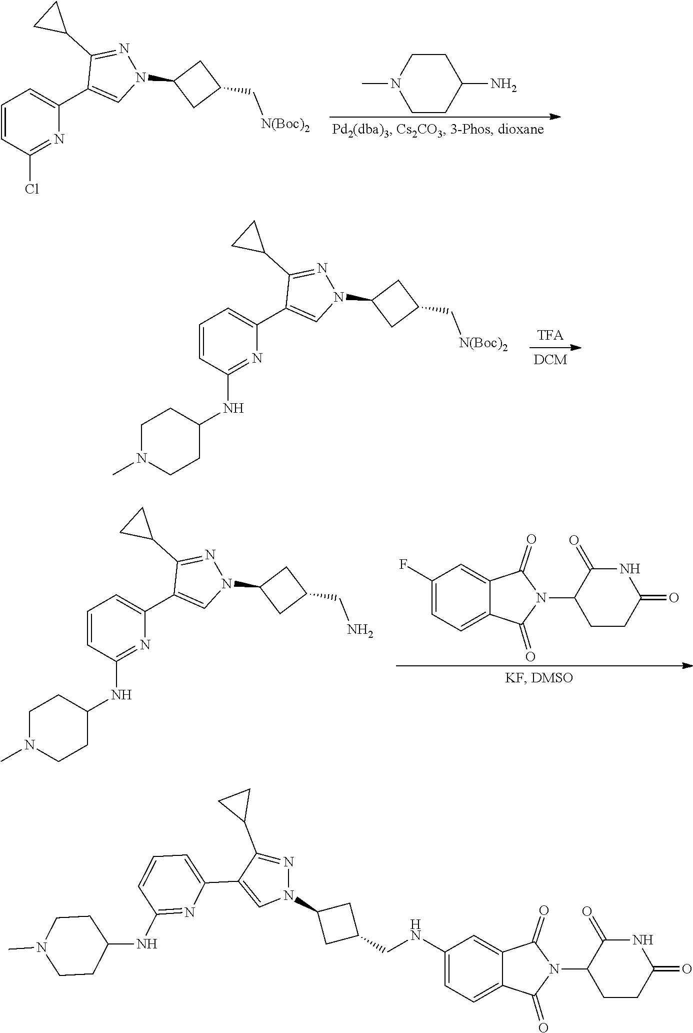

GS-454 was synthesized following the standard procedures for preparing GS-440 (4.1 mg, 21% yield) as a yellow solid. MS (ESI) m/z=637.3 [M+H]⁺.

### Example 413. 5-(((trans-3-(3-Cyclopropyl-4-(piperazin-1-yl)-1H-pyrazol-1-yl)cyclobutyl)methyl)amino)-2-(2,6-dioxopiperidin-3-yl)isoindoline-1,3-dione (GS-455)

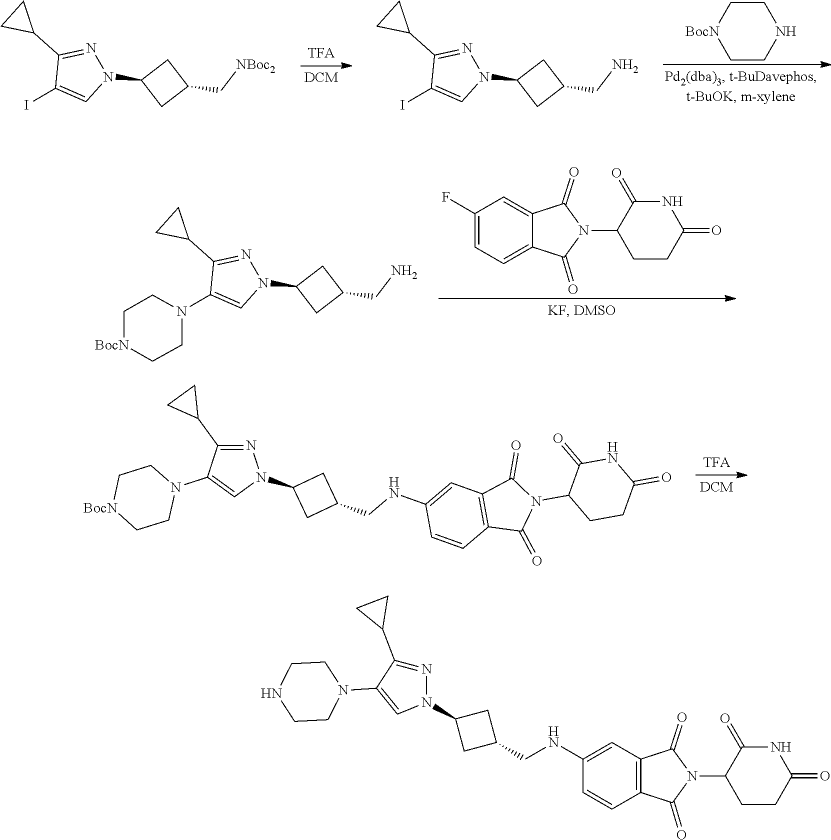

GS-455 was synthesized following the standard procedures for preparing GS-438 (7.3 mg, 28.5% yield) as a light-yellow solid. MS (ESI) m/z=532.4 [M+H]⁺.

### Example 414. 5-(((trans-3-(4-(4-Chlorophenyl)-3-cyclopropyl-1H-pyrazol-1-yl)cyclobutyl)methyl)amino)-2-(2,6-dioxopiperidin-3-yl)isoindoline-1,3-dione (GS-456)

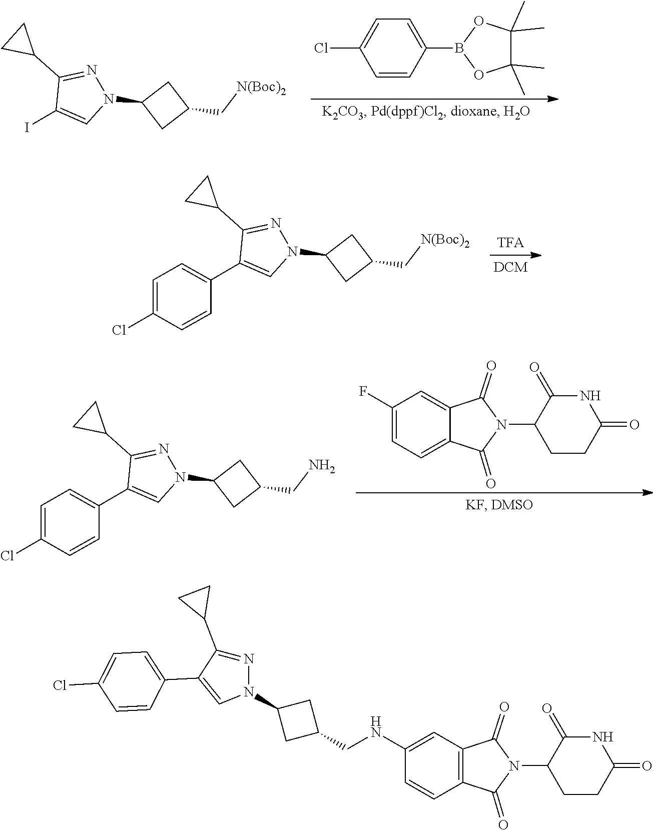

Step 1. Synthesis of tert-butyl-N-tert-butoxycarbonyl-N-[[trans-3-[4-(4-chlorophenyl)-3-cyclopropyl-pyrazol-1-yl]cyclobutyl]methyl]carbamate

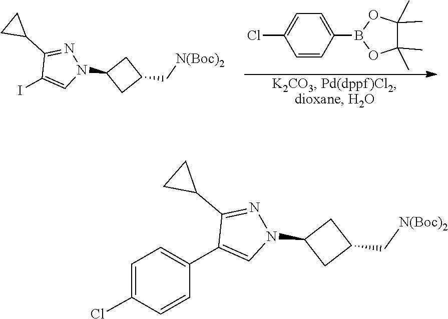

To a mixture of 2-(4-chlorophenyl)-4,4,5,5-tetramethyl-1,3,2-dioxaborolane (23.8 mg, 0.1 mmol) and tert-butyl-N-tert-butoxycarbonyl-N-[[trans-3-[3-cyclopropyl-4-iodo-pyrazol-1-yl]cyclobutyl]methyl]carbamate (51.8 mg, 0.1 mmol) in dioxane (9 mL) and H₂O (1 mL) were added K₂CO₃ (42.0 mg, 0.3 mmol) and Pd(dppf)Cl₂ (21.9 mg, 0.03 mmol) at rt. The reaction mixture was stirred at 100° C. overnight. After cooling down to rt, the reaction was quenched with H₂O (10 mL) and extracted with EtOAc (3×20 mL). The combined organic layers were dried over anhydrous sodium sulfate, filtered, and concentrated under reduced pressure. The resulting residue was purified by reverse-phase chromatography to provide the desired product (30.2 mg, 60% yield) as a light-yellow solid. MS (ESI) m/z=502.3 [M+H]⁺.

The remaining steps were performed according to the standard procedures for preparing GS-438 to provide the desired product (6.30 mg, 19% yield over 2 steps) as a light-yellow solid. MS (ESI) m/z=558.3 [M+H]⁺.

### Example 415. 5-(((trans-3-(4-(3-Chlorophenyl)-3-cyclopropyl-1H-pyrazol-1-yl)cyclobutyl)methyl)amino)-2-(2,6-dioxopiperidin-3-yl)isoindoline-1,3-dione (GS-457)

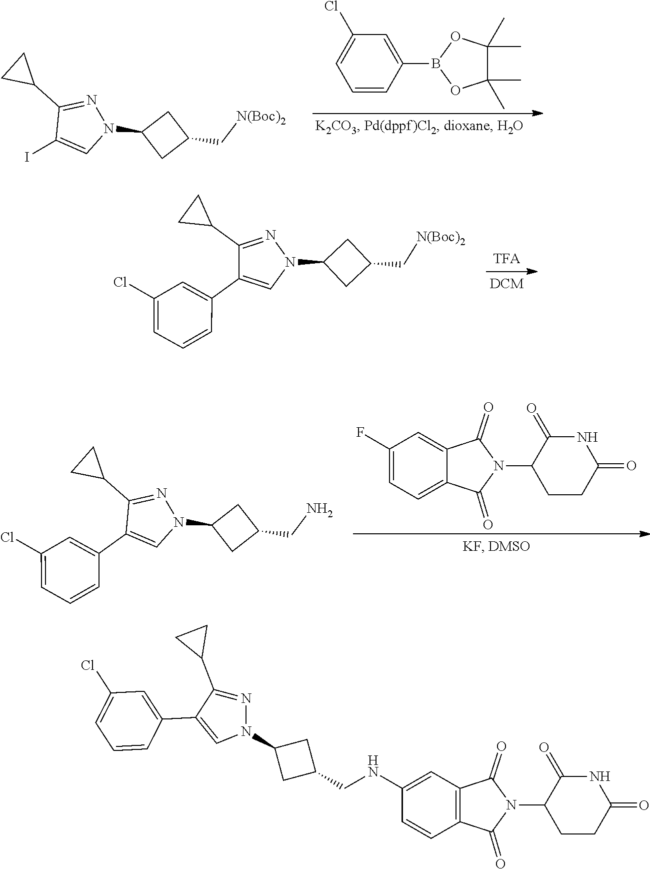

GS-457 was synthesized following the standard procedures for preparing GS-456 and GS-438 (6.2 mg, 18% yield over 3 steps) as a light-yellow solid. MS (ESI) m/z=558.3 [M+H]⁺.

### Example 416. 5-(((trans-3-(4-(2-Chlorophenyl)-3-cyclopropyl-1H-pyrazol-1-yl)cyclobutyl)methyl)amino)-2-(2,6-dioxopiperidin-3-yl)isoindoline-1,3-dione (GS-458)

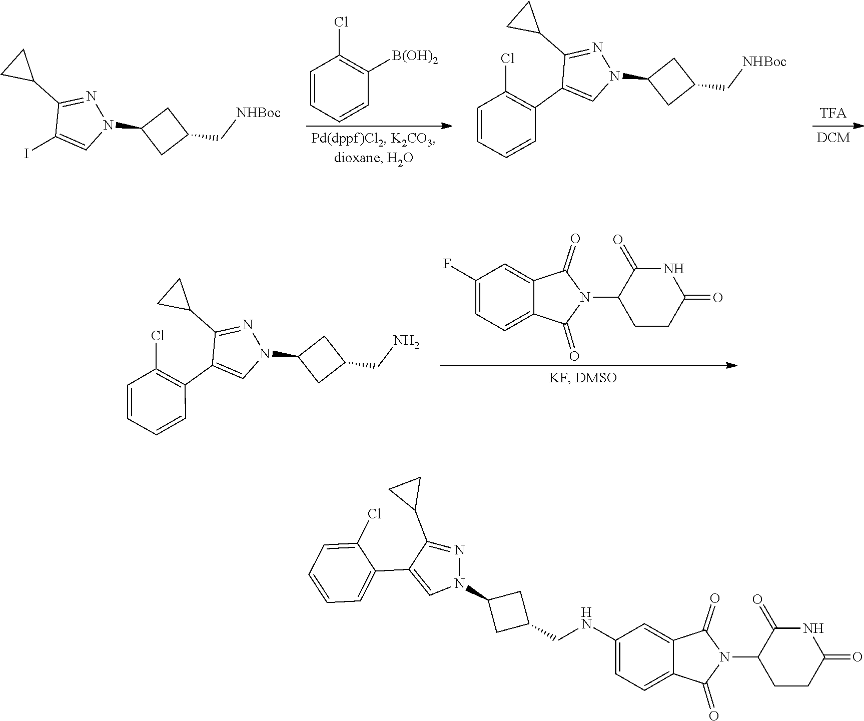 [SMILES: CC(C)(C)OC(=O)NC[C@H]1C[C@H](n2cc(I)c(C3CC3)n2)C1 ; OB(O)c1ccccc1Cl ; CC(C)(C)OC(=O)NC[C@H]1C[C@H](n2cc(-c3ccccc3Cl)c(C3CC3)n2)C1 ; NC[C@H]1C[C@H](n2cc(-c3ccccc3Cl)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6ccccc6Cl)c(C6CC6)n5)C4)cc3C2=O)C(=O)N1]

GS-458 was synthesized following the standard procedures for preparing GS-456 and GS-438 (1.6 mg, 11% yield over 3 steps) as a light-yellow solid. MS (ESI) m/z=558.3 [M+H]⁺.

### Example 417. 5-(((trans-3-(3-Cyclopropyl-4-(4-methylpiperazin-1-yl)-1H-pyrazol-1-yl)cyclobutyl)methyl)amino)-2-(2,6-dioxopiperidin-3-yl)isoindoline-1,3-dione (GS-459)

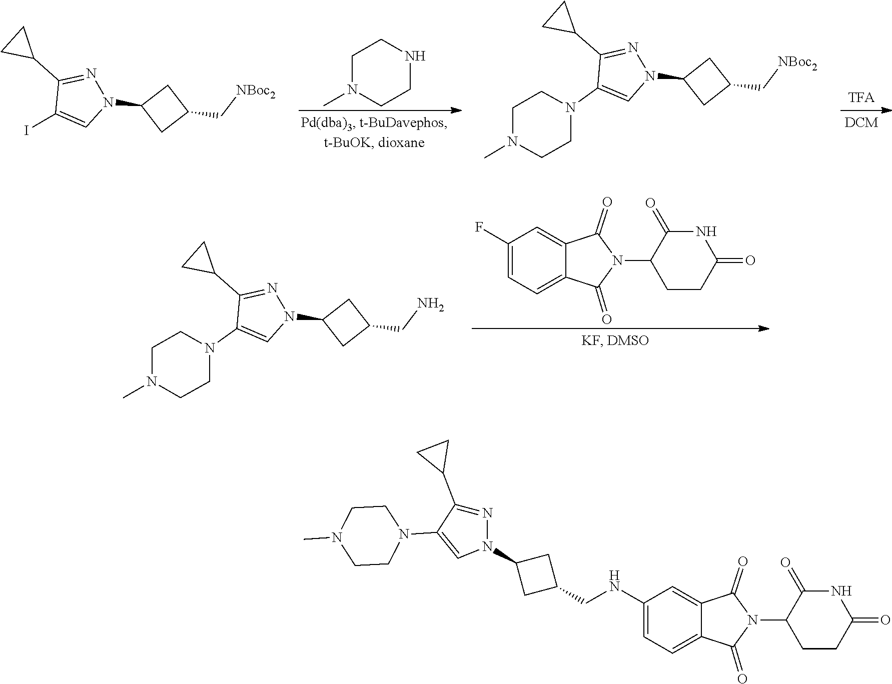

GS-459 was synthesized following the standard procedures for preparing GS-438 (1.3 mg, 10% yield over 3 steps) as a light-yellow solid. MS (ESI) m/z=546.4 [M+H]⁺.

### Example 418. 5-(((trans-3-(3-Cyclopropyl-4-(1-methylpiperidin-4-yl)-1H-pyrazol-1-yl)cyclobutyl)methyl)amino)-2-(2,6-dioxopiperidin-3-yl)isoindoline-1,3-dione (GS-460)

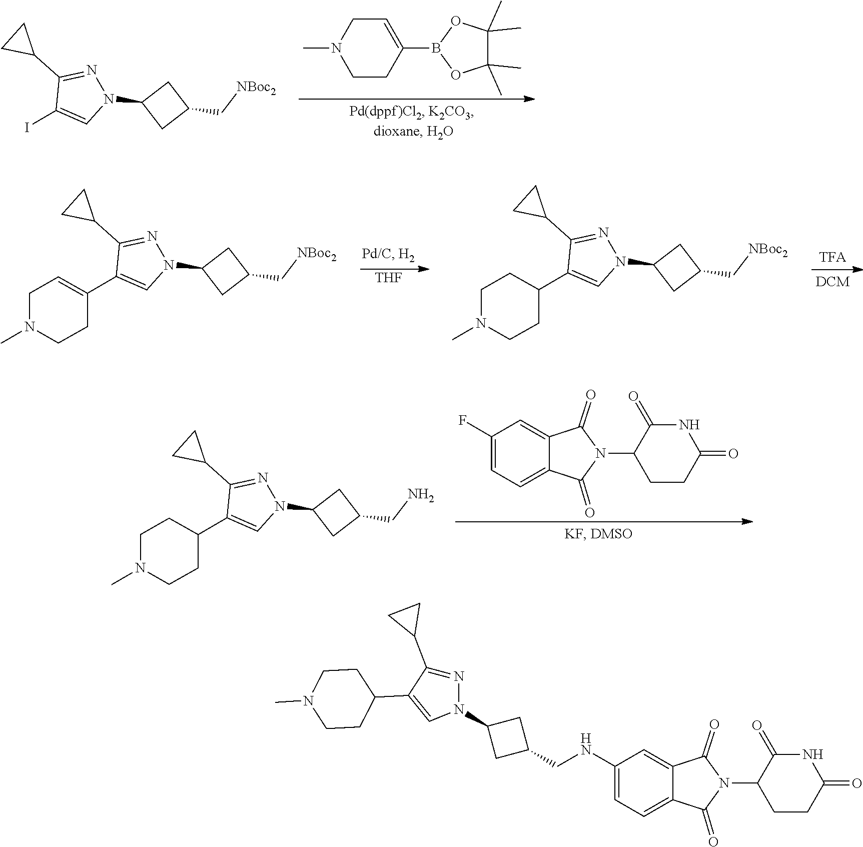

Step 1. Synthesis of tert-butyl N-tert-butoxycarbonyl-N-[[trans-3-[3-cyclopropyl-4-(1-methyl-3,6-dihydro-2H-pyridin-4-yl)pyrazol-1-yl]cyclobutyl]methyl]carbamate

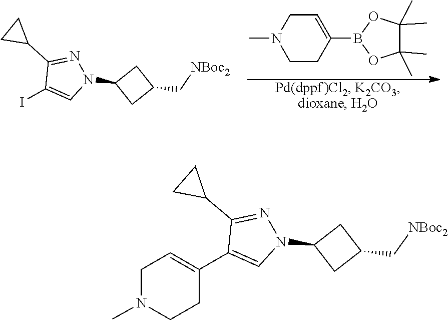

To a solution of tert-butyl N-tert-butoxycarbonyl-N-[[trans-3-(3-cyclopropyl-4-iodo-pyrazol-1-yl)cyclobutyl]methyl]carbamate (40.0 mg, 77.3 μmol) in dioxane (5 mL) and H₂O (1 mL) were added 1-methyl-4-(4,4,5,5-tetramethyl-1,3,2-dioxaborolan-2-yl)-3,6-dihydro-2H-pyridine (20.7 mg, 0.093 mmol), Pd(dppf)Cl₂ (2.8 mg, 0.004 mmol) and K₂CO₃ (21.3 mg, 0.15 mmol) at rt. Then the mixture was stirred at 100° C. under inert atmosphere for 0.5 h. After cooling down to rt, the solution was poured into water (10 mL) and extracted with ethyl acetate (3×10 mL). The combined organic layers were washed with saturated brine (15 mL), dried over anhydrous sodium sulfate, filtered, and concentrated under reduced pressure. The resulting residue was purified by reverse-phase chromatography to provide the desired product (20.4 mg, 53% yield) as a light-yellow solid. MS (ESI) m/z=487.4 [M+H]⁺.

Step 2. Synthesis of tert-butyl N-tert-butoxycarbonyl-N-[[trans-3-[3-cyclopropyl-4-(1-methyl-4-piperidyl)pyrazol-1-yl]cyclobutyl]methyl]carbamate

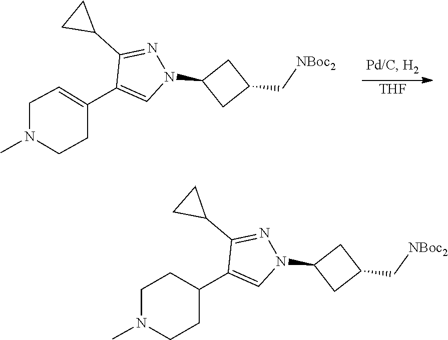

To a solution of tert-butyl-N-tert-butoxycarbonyl-N-[[trans-3-[3-cyclopropyl-4-(1-methyl-3,6-dihydro-2H-pyridin-4-yl)pyrazol-1-yl]cyclobutyl]methyl]carbamate (20.4 mg, 0.041 mmol) in THF (4 mL) was added 10% Pd/C (3.3 mg). The reaction mixture was purged with hydrogen and stirred at rt under hydrogen balloon for 2 h. The solution was filtered through Celite and the filtrate was concentrated and purified by reverse-phase chromatography to provide the desired product (15.0 mg, 75% yield). MS (ESI) m/z=489.4 [M+H]⁺.

The remaining steps were performed according to the standard procedures for preparing GS-438 to provide the desired product (1.5 mg, 9% yield over 2 steps) as a yellow solid. MS (ESI) m/z=545.4 [M+H]⁺.

### Example 419. 5-(((3-(3-Cyclopropyl-4-(quinoxalin-2-yl)-1H-pyrazol-1-yl)cyclobutyl)amino)methyl)-2-(2,6-dioxopiperidin-3-yl)isoindoline-1,3-dione (GS-461)

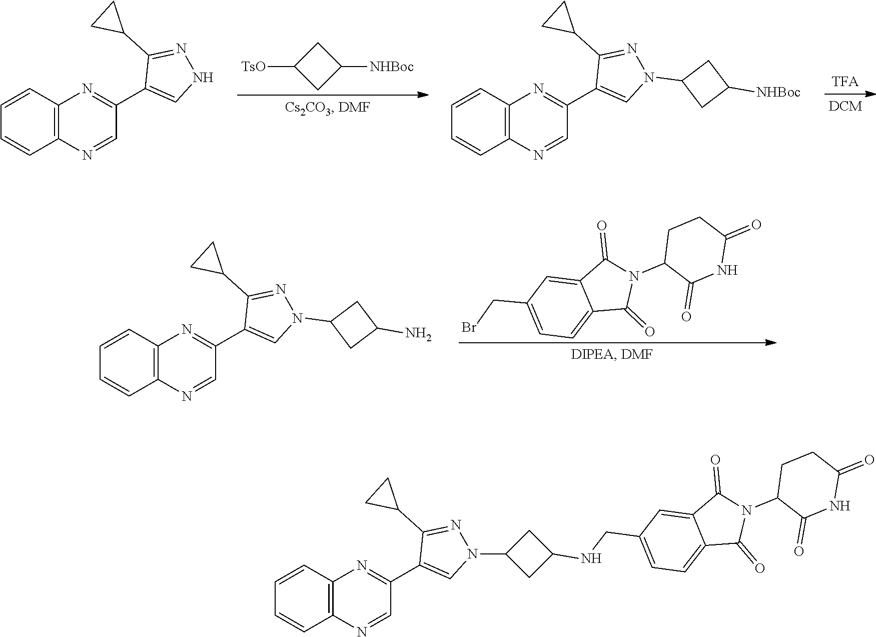 [SMILES: c1ccc2nc(-c3c[nH]nc3C3CC3)cnc2c1 ; Cc1ccc(S(=O)(=O)OC2CC(NC(=O)OC(C)(C)C)C2)cc1 ; CC(C)(C)OC(=O)NC1CC(n2cc(-c3cnc4ccccc4n3)c(C3CC3)n2)C1 ; NC1CC(n2cc(-c3cnc4ccccc4n3)c(C3CC3)n2)C1 ; O=C1CCC(N2C(=O)c3ccc(CBr)cc3C2=O)C(=O)N1 ; O=C1CCC(N2C(=O)c3ccc(CNC4CC(n5cc(-c6cnc7ccccc7n6)c(C6CC6)n5)C4)cc3C2=O)C(=O)N1]

Step 1. Synthesis of tert-butyl (3-(3-cyclopropyl-4-(quinoxalin-2-yl)-1H-pyrazol-1-yl)cyclobutyl)carbamate

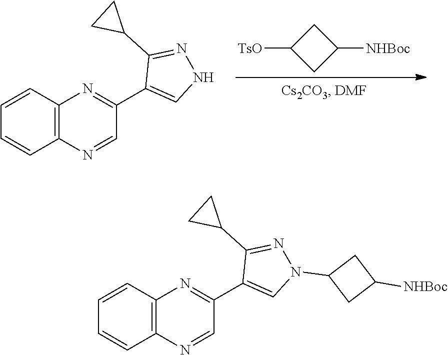 [SMILES: c1ccc2nc(-c3c[nH]nc3C3CC3)cnc2c1 ; Cc1ccc(S(=O)(=O)OC2CC(NC(=O)OC(C)(C)C)C2)cc1 ; CC(C)(C)OC(=O)NC1CC(n2cc(-c3cnc4ccccc4n3)c(C3CC3)n2)C1]

A mixture of 2-(3-cyclopropyl-1H-pyrazol-4-yl)quinoxaline (100 mg, 0.42 mmol), 3-((tert-butoxycarbonyl)amino)cyclobutyl 4-methylbenzenesulfonate (164 mg, 0.48 mmol) and Cs₂CO₃ (430 mg, 1.32 mmol) in DMF (3 mL) was stirred at 100° C. for 2 h under N₂. The solution was poured into water (10 mL) and extracted with ethyl acetate (3×10 mL). The combined organic layers were washed with saturated brine (10 mL), dried over anhydrous sodium sulfate, filtered, and concentrated under reduced pressure. The resulting residue was purified by silica gel column chromatography to provide the desired product (112 mg, 66% yield) as a light-yellow solid. MS (ESI) m/z=406.2 [M+H]⁺.

Step 2. Synthesis of 3-(3-cyclopropyl-4-(quinoxalin-2-yl)-1H-pyrazol-1-yl)cyclobutan-1-amine

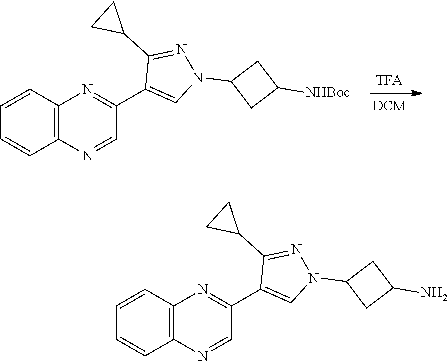 [SMILES: CC(C)(C)OC(=O)NC1CC(n2cc(-c3cnc4ccccc4n3)c(C3CC3)n2)C1 ; NC1CC(n2cc(-c3cnc4ccccc4n3)c(C3CC3)n2)C1]

A mixture of tert-butyl (3-(3-cyclopropyl-4-(quinoxalin-2-yl)-1H-pyrazol-1-yl)cyclobutyl)carbamate (110 mg, 0.27 mmol) in TFA (2 mL) and DCM (2 mL) was stirred at rt for 1 h. The reaction mixture was concentrated and purified by reverse-phase chromatography to provide the desired product (60 mg, 73% yield) as a light-yellow solid. MS (ESI) m/z=306.2 [M+H]⁺.

Step 3. Synthesis of 5-(((3-(3-cyclopropyl-4-(quinoxalin-2-yl)-1H-pyrazol-1-yl)cyclobutyl)amino)methyl)-2-(2,6-dioxopiperidin-3-yl)isoindoline-1,3-dione

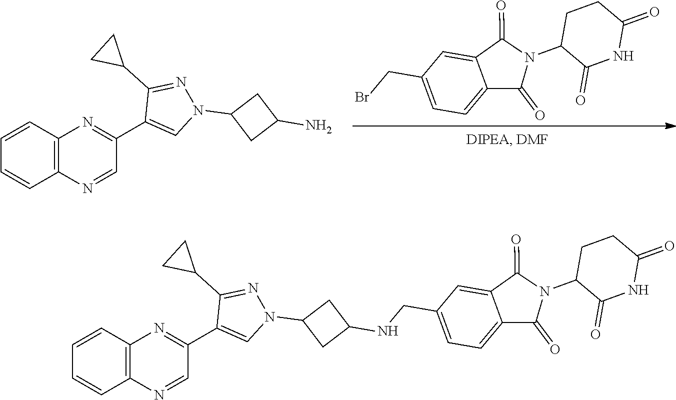 [SMILES: NC1CC(n2cc(-c3cnc4ccccc4n3)c(C3CC3)n2)C1 ; O=C1CCC(N2C(=O)c3ccc(CBr)cc3C2=O)C(=O)N1 ; O=C1CCC(N2C(=O)c3ccc(CNC4CC(n5cc(-c6cnc7ccccc7n6)c(C6CC6)n5)C4)cc3C2=O)C(=O)N1]

A mixture of 3-(3-cyclopropyl-4-(quinoxalin-2-yl)-1H-pyrazol-1-yl)cyclobutan-1-amine (10 mg, 0.03 mmol), 5-(bromomethyl)-2-(2,6-dioxopiperidin-3-yl)isoindoline-1,3-dione (10 mg, 0.03 mmol) and DIPEA (7.7 mg, 0.06 mmol) in DMF (2 mL) was stirred at 80° C. for 2 h under N₂. The solution was poured into water (10 mL) and extracted with ethyl acetate (3×10 mL). The combined organic layers were washed with saturated brine (10 mL), dried over anhydrous sodium sulfate, filtered, and concentrated under reduced pressure. The resulting residue was purified by prep-TLC to provide the desired product (5.2 mg, 31% yield) as a light-yellow solid. MS (ESI) m/z=576.2 [M+H]⁺.

### Example 420. 2-(2,6-Dioxopiperidin-3-yl)-5-((3-(trans-3-(4-(5-methoxypyridin-2-yl)-1H-pyrazol-1-yl)cyclobutyl)propyl)amino)isoindoline-1,3-dione (GS-462)

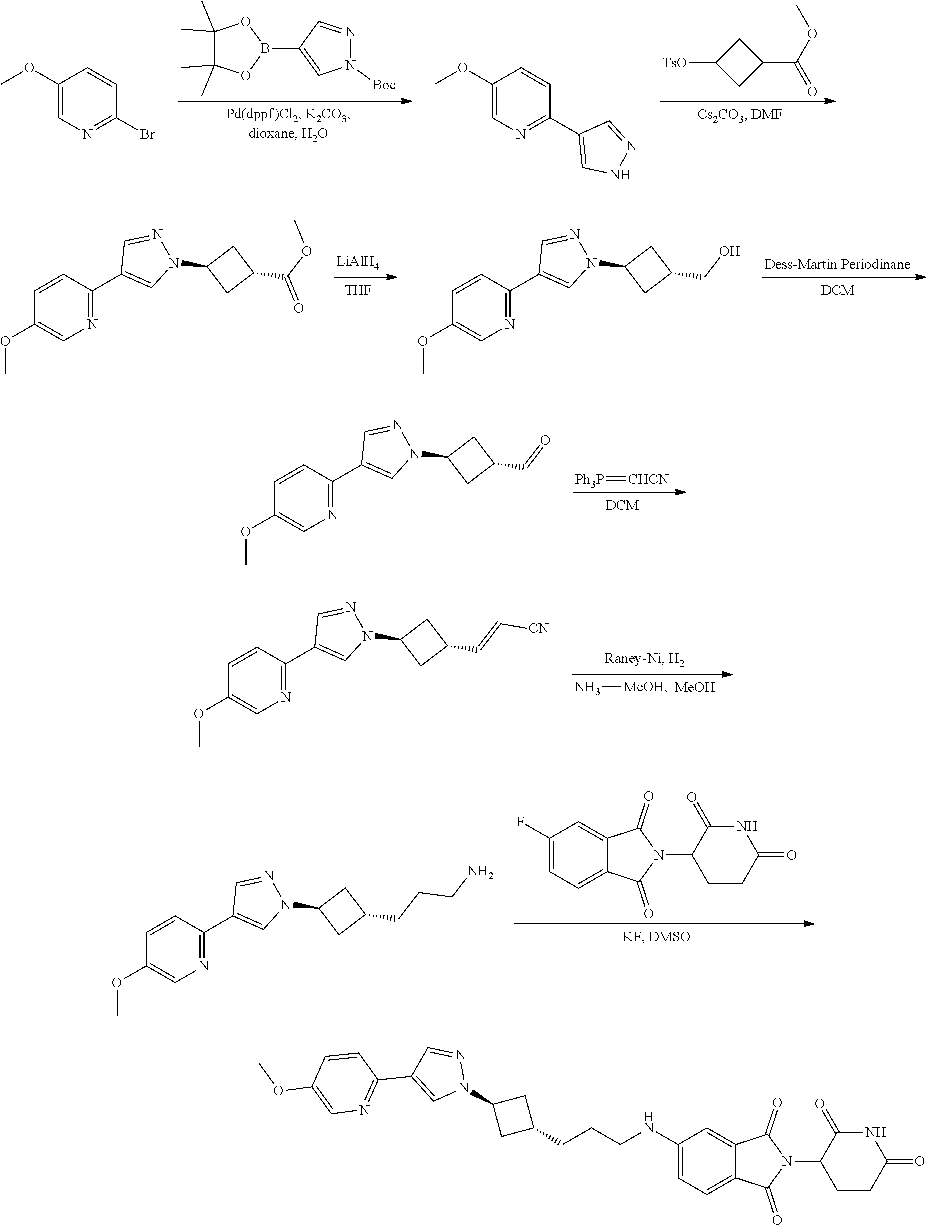

GS-462 was synthesized following the standard procedures for preparing GS-399 (1.8 mg, 10% yield) as a light-yellow solid. MS (ESI) m/z=543.3 [M+H]⁺.

### Example 421. 5-(((trans-3-(3-Cyclopropyl-4-(7-(4-methylpiperazin-1-yl)quinoxalin-2-yl)-1H-pyrazol-1-yl)cyclobutyl)methyl)amino)-2-(2,6-dioxopiperidin-3-yl)isoindoline-1,3-dione (GS-463)

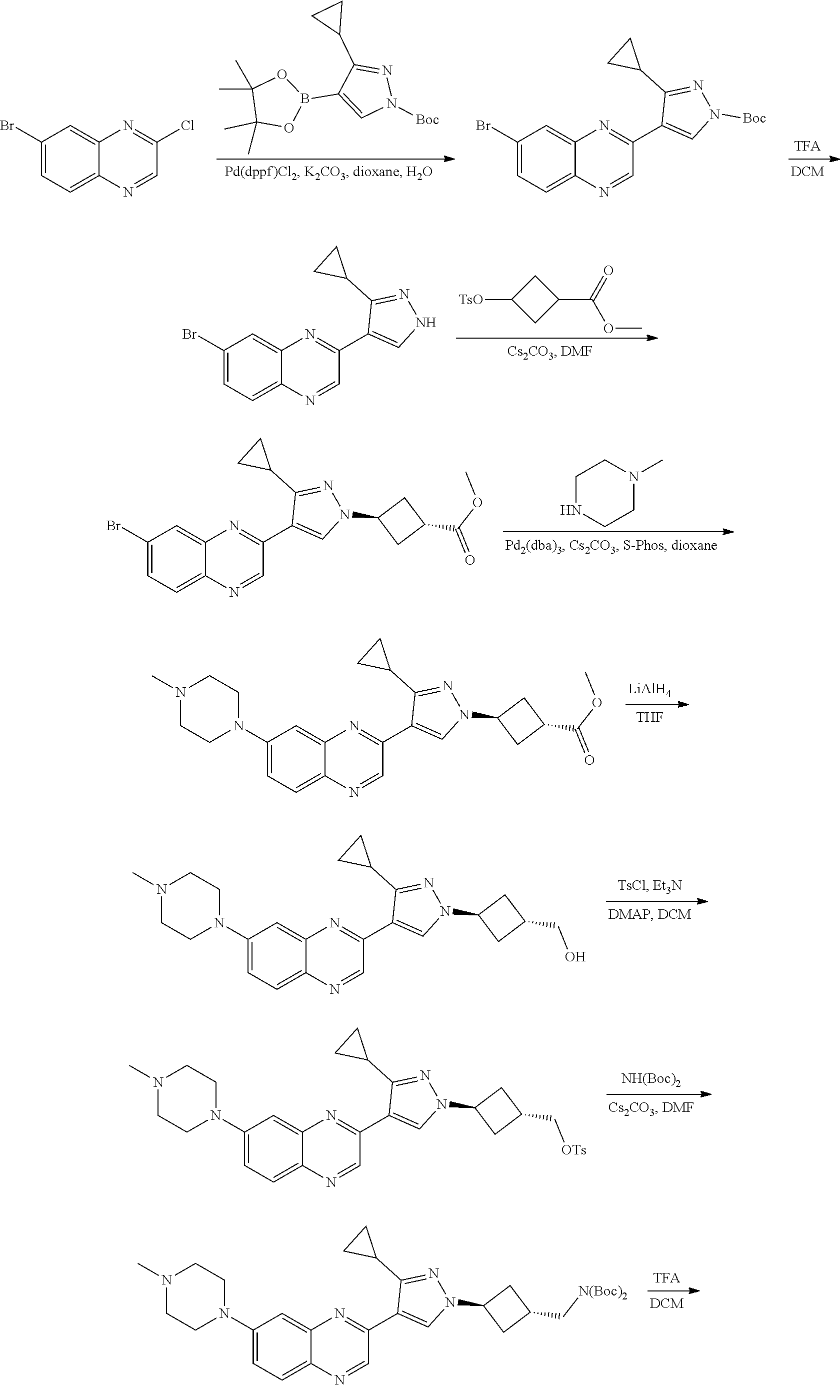

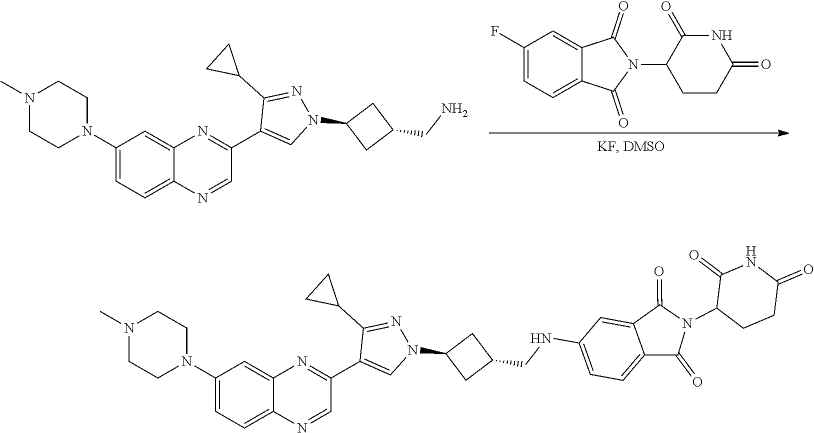 [SMILES: CN1CCN(c2ccc3ncc(-c4cn([C@H]5C[C@H](CN)C5)nc4C4CC4)nc3c2)CC1 ; O=C1CCC(N2C(=O)c3ccc(F)cc3C2=O)C(=O)N1 ; CN1CCN(c2ccc3ncc(-c4cn([C@H]5C[C@H](CNc6ccc7c(c6)C(=O)N(C6CCC(=O)NC6=O)C7=O)C5)nc4C4CC4)nc3c2)CC1]

GS-463 was synthesized following the procedures or preparing GS-399 an GS-442 to provide the desired product (9 mg, 19% yield) as a yellow solid. MS (ESI) m/z=674.4 [M+H]⁺.

### Example 422. 5-(((trans-3-(3-Cyclopropyl-4-(7-morpholinoquinoxalin-2-yl)-1H-pyrazol-1-yl)cyclobutyl)methyl)amino)-2-(2,6-dioxopiperidin-3-yl)isoindoline-1,3-dione (GS-464)

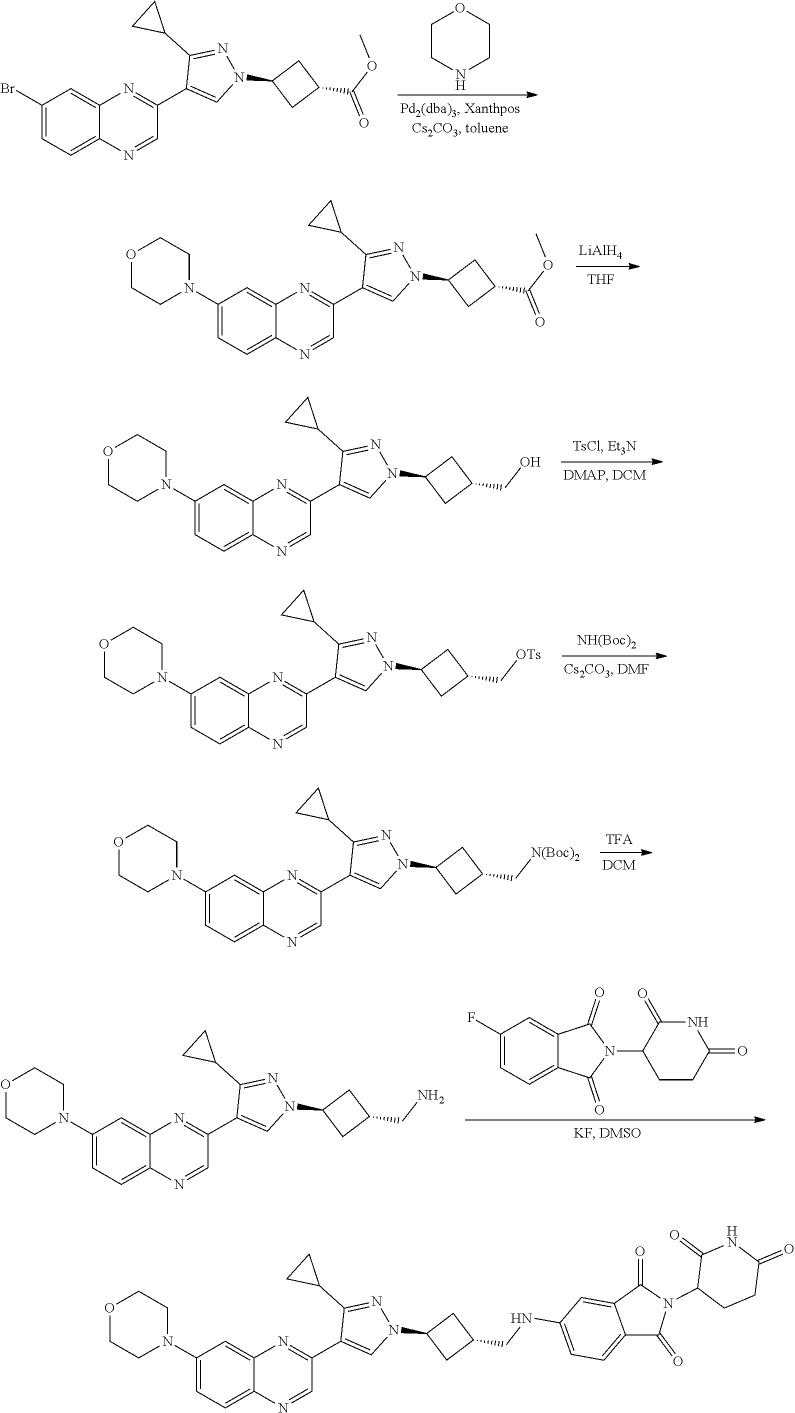

GS-464 was synthesized following the procedures for preparing GS-399 and GS-442 (5 mg, 5% yield over 6 steps) as a yellow solid. MS (ESI) m/z=661.3 [M+H]⁺.

### Example 423. 5-(((trans-3-(3-Cyclopropyl-4-(1-methyl-2-oxo-1,2-dihydropyridin-3-yl)-1H-pyrazol-1-yl)cyclobutyl)methyl)amino)-2-(2,6-dioxopiperidin-3-yl)isoindoline-1,3-dione (GS-465)

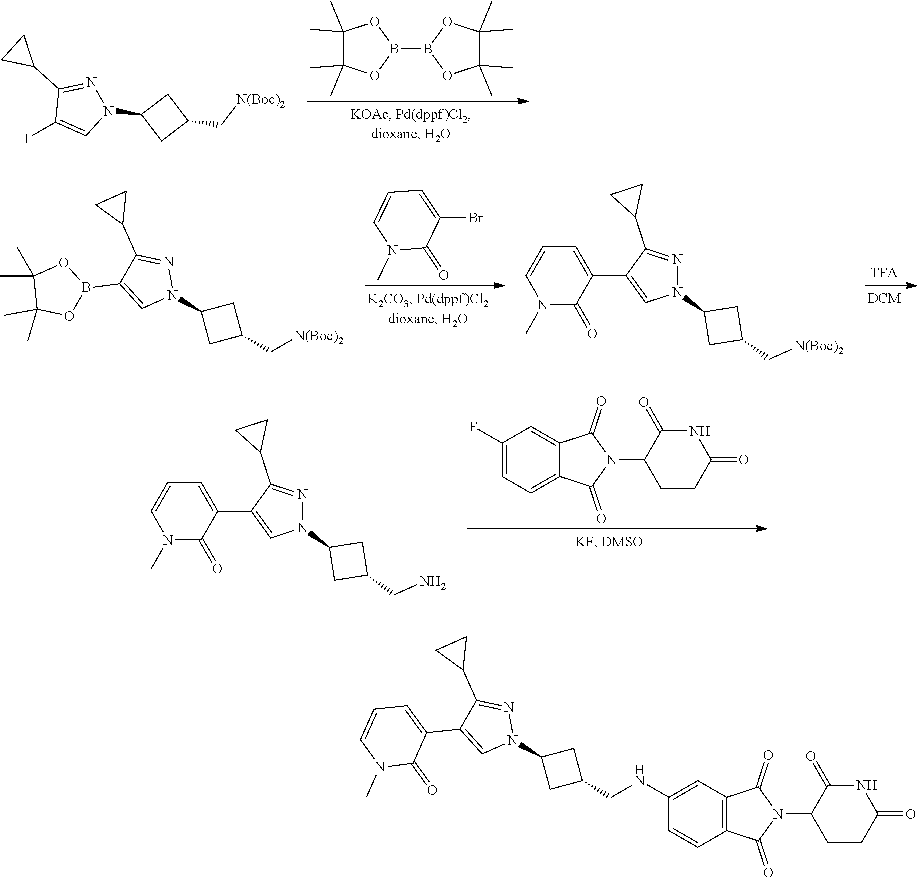

GS-465 was synthesized following the standard procedures for preparing GS-456 and GS-438 (6.0 mg, 28% yield) as a light-yellow solid. MS (ESI) m/z=555.3 [M+H]⁺.

### Example 424. 5-(((trans-3-(3-Cyclopropyl-4-(6-cyclopropylpyridin-2-yl)-1H-pyrazol-1-yl)cyclobutyl)methyl)amino)-2-(2,6-dioxopiperidin-3-yl)isoindoline-1,3-dione (GS-466)

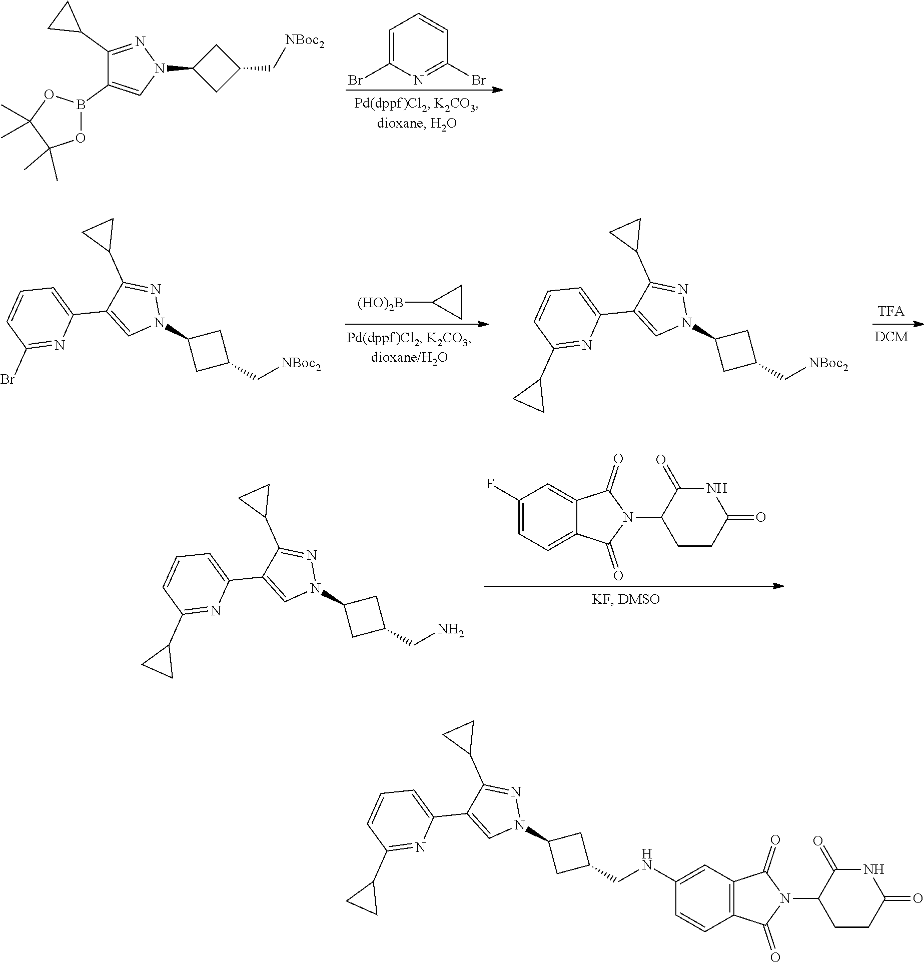

GS-466 was synthesized following the standard procedures for preparing GS-456 and GS-438 (1.3 mg, 2% yield over 4 steps) as a light-yellow solid. MS (ESI) m/z=565.3 [M+H]⁺.

### Example 425. 5-(((trans-3-(3-Cyclopropyl-4-(3-cyclopropylpyridin-2-yl)-1H-pyrazol-1-yl)cyclobutyl)methyl)amino)-2-(2,6-dioxopiperidin-3-yl)isoindoline-1,3-dione (GS-467)

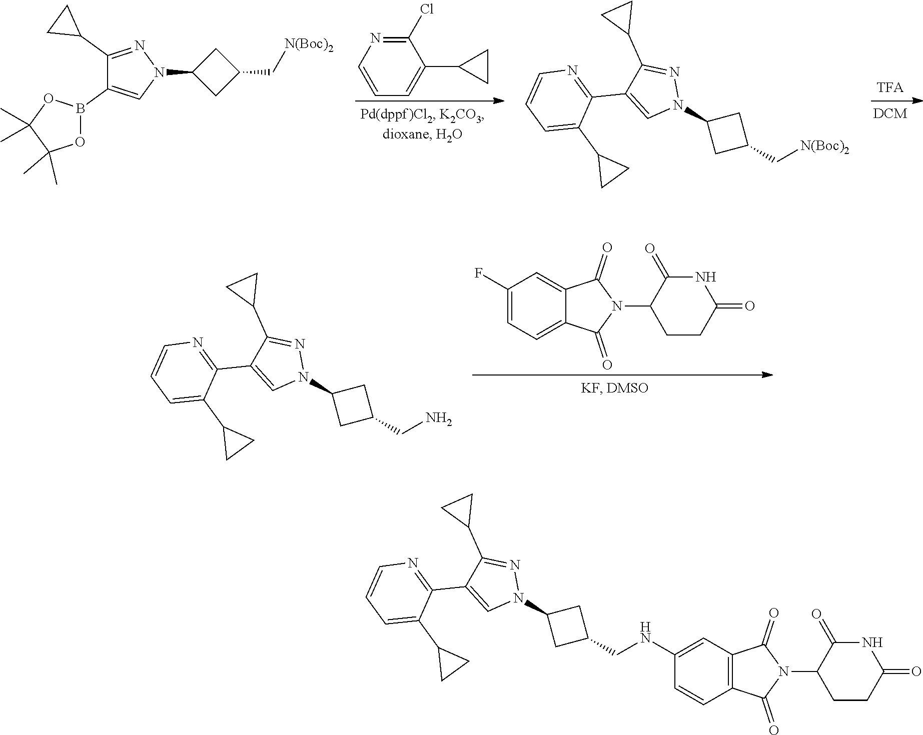

GS-467 was synthesized following the standard procedures for preparing GS-456 and GS-438 (1.26 mg, 7% yield over 3 steps) as a yellow solid. MS (ESI) m/z=565.3 [M+H]⁺.

### Example 426. 5-(((trans-3-(4-Cyclohexyl-3-cyclopropyl-1H-pyrazol-1-yl)cyclobutyl)methyl)amino)-2-(2,6-dioxopiperidin-3-yl)isoindoline-1,3-dione (GS-468)

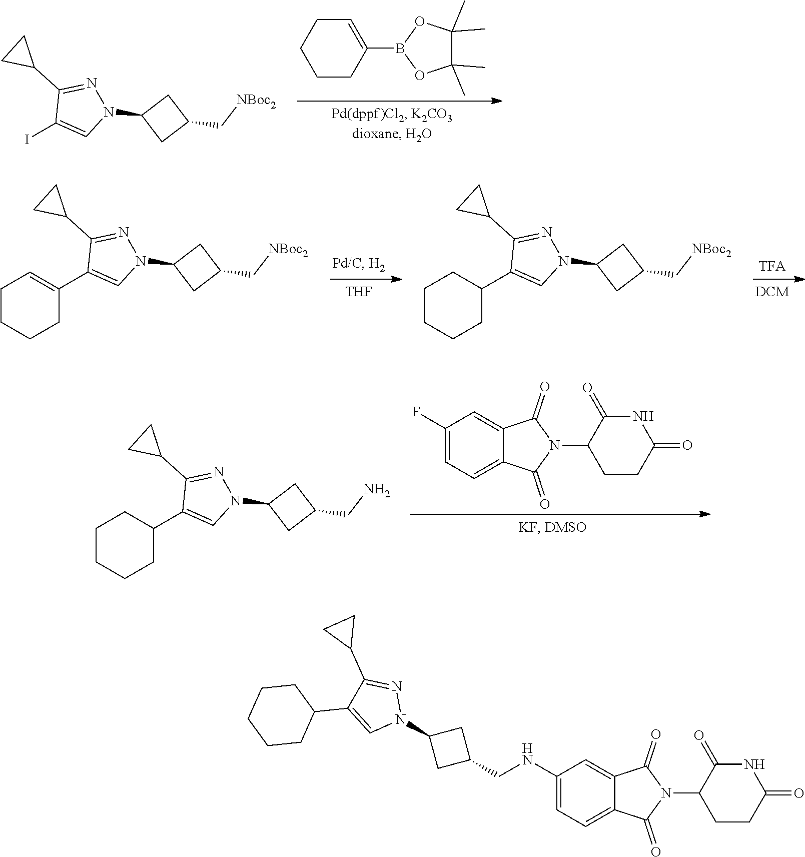

GS-468 was synthesized following the standard procedures for preparing GS-460 (1.8 mg, 14% yield) as a light-yellow solid. MS (ESI) m/z=530.3 [M+H]⁺.

### Example 427. 5-(2-(3-(3-Cyclopropyl-4-(quinoxalin-2-yl)-1H-pyrazol-1-yl)azetidin-1-yl)ethyl)-2-(2,6-dioxopiperidin-3-yl)isoindoline-1,3-dione (GS-469)

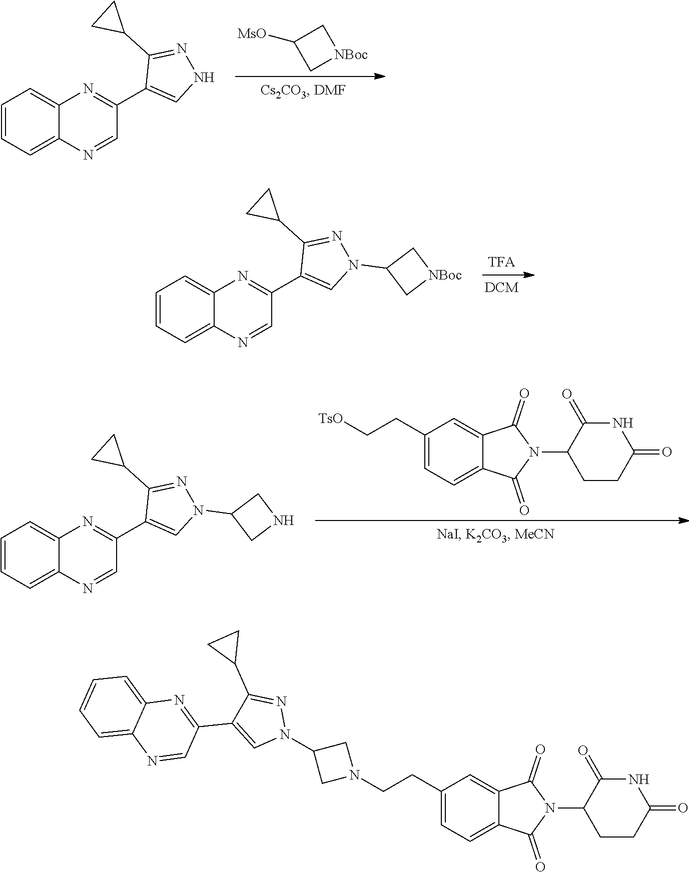 [SMILES: c1ccc2nc(-c3c[nH]nc3C3CC3)cnc2c1 ; CC(C)(C)OC(=O)N1CC(OS(C)(=O)=O)C1 ; CC(C)(C)OC(=O)N1CC(n2cc(-c3cnc4ccccc4n3)c(C3CC3)n2)C1 ; c1ccc2nc(-c3cn(C4CNC4)nc3C3CC3)cnc2c1 ; Cc1ccc(S(=O)(=O)OCCc2ccc3c(c2)C(=O)N(C2CCC(=O)NC2=O)C3=O)cc1 ; O=C1CCC(N2C(=O)c3ccc(CCN4CC(n5cc(-c6cnc7ccccc7n6)c(C6CC6)n5)C4)cc3C2=O)C(=O)N1]

Step 1 and step 2 followed the procedures for preparing GS-461 to provide the desired product (310 mg, 53% yield over 2 steps) as a red brown solid. MS (ESI) m/z=292.1 [M+H]⁺.

Step 3. Synthesis of 5-(2-(3-(3-cyclopropyl-4-(quinoxalin-2-yl)-1H-pyrazol-1-yl)azetidin-1-yl)ethyl)-2-(2,6-dioxopiperidin-3-yl)isoindoline-1,3-dione

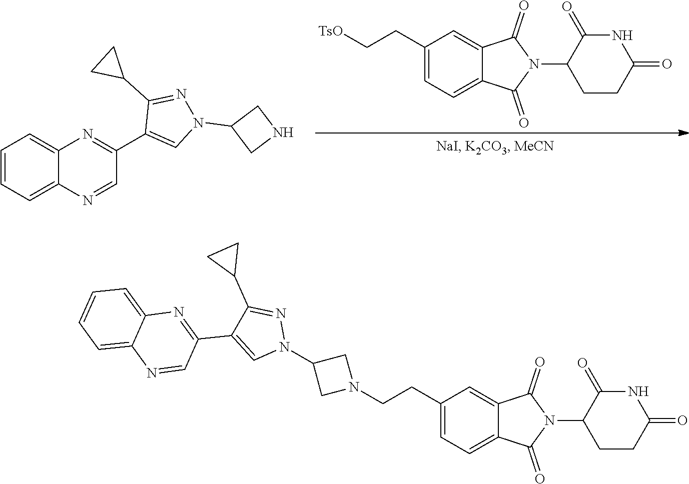 [SMILES: c1ccc2nc(-c3cn(C4CNC4)nc3C3CC3)cnc2c1 ; Cc1ccc(S(=O)(=O)OCCc2ccc3c(c2)C(=O)N(C2CCC(=O)NC2=O)C3=O)cc1 ; O=C1CCC(N2C(=O)c3ccc(CCN4CC(n5cc(-c6cnc7ccccc7n6)c(C6CC6)n5)C4)cc3C2=O)C(=O)N1]

To a solution of 2-(1-(azetidin-3-yl)-3-cyclopropyl-1H-pyrazol-4-yl)quinoxaline (15.0 mg, 0.05 mmol) and 2-(2-(2,6-dioxopiperidin-3-yl)-1,3-dioxoisoindolin-5-yl)ethyl 4-methylbenzenesulfonate (22.8 mg, 0.05 mmol) in MeCN (3 mL) were added K₂CO₃ (27.6 mg, 0.20 mmol) and NaI (14.9 mg, 0.10 mmol) at rt. The reaction mixture was stirred at 90° C. for 10 h. Then the mixture was purified by reverse-phase chromatography to provide the desired product (6.4 mg, 43% yield) as a white solid. MS (ESI) m/z 576.4 [M+H]⁺.

### Example 428. 5-(((trans-3-(3-Cyclopropyl-1H-pyrazolo[3,4-b]pyridin-1-yl)cyclobutyl)methyl)amino)-2-(2,6-dioxopiperidin-3-yl)isoindoline-1,3-dione (GS-470)

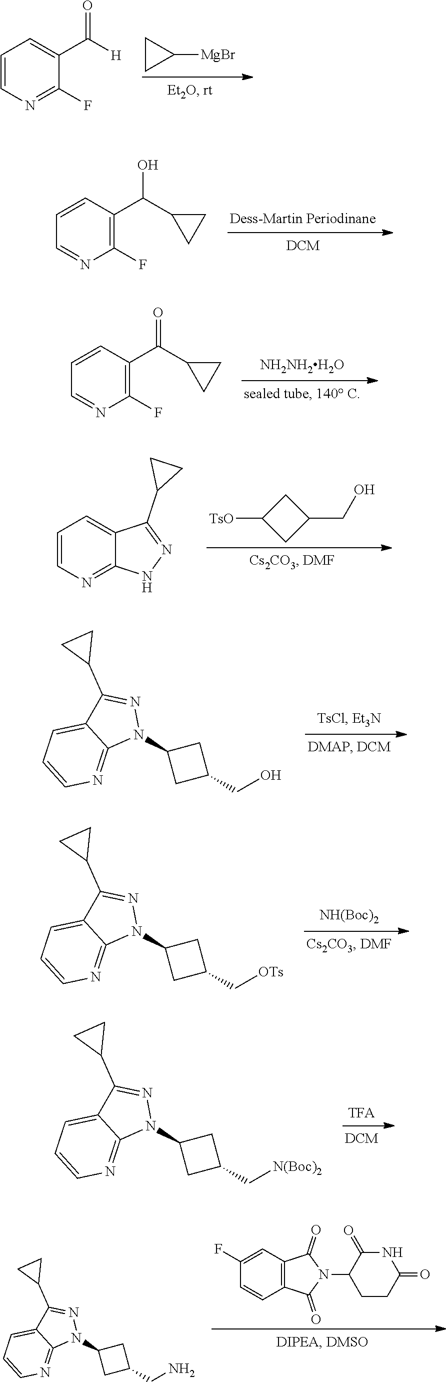

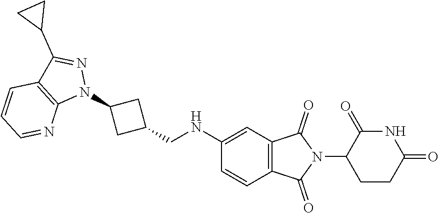 [SMILES: O=C1CCC(N2C(=O)c3ccc(NC[C@H]4C[C@H](n5nc(C6CC6)c6cccnc65)C4)cc3C2=O)C(=O)N1]

GS-470 was synthesized following the standard procedures for preparing GS-442 (4.2 mg, 2% yield over 8 steps) as a light-yellow solid. MS (ESI) m/z=499.3 [M+H]⁺.

### Example 429. 5-(((trans-3-(4-(6-Cyclopropoxypyridin-2-yl)-3-cyclopropyl-1H-pyrazol-1-yl)cyclobutyl)methyl)amino)-2-(2,6-dioxopiperidin-3-yl)isoindoline-1,3-dione (GS-471)

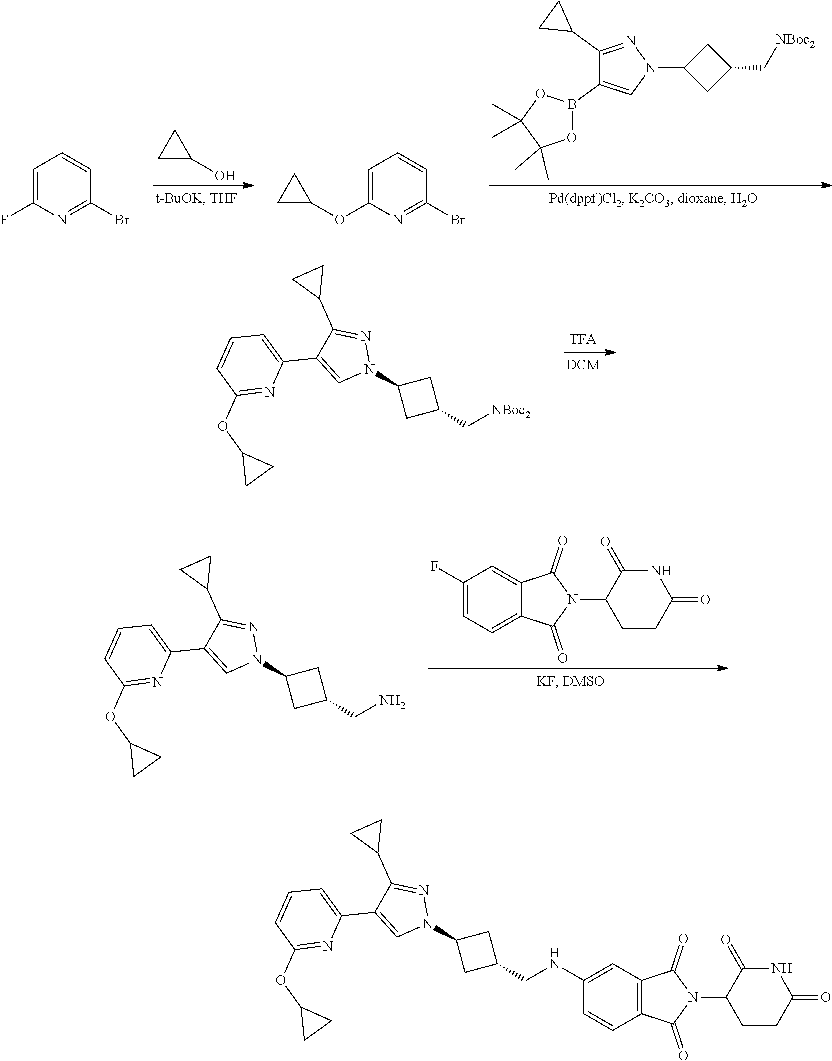

Step 1. Synthesis of 2-bromo-6-cyclopropoxypyridine

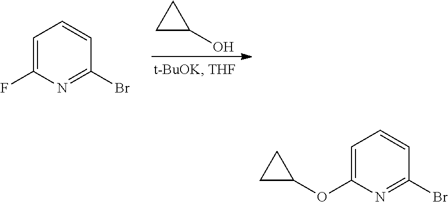

To a solution of 2-bromo-6-fluoro-pyridine (300.0 mg, 1.70 mmol) and cyclopropanol (99.0 mg, 1.70 mmol) in THF (10 mL) was added t-BuOK (191.3 mg, 1.70 mmol) at 0° C. Then the reaction mixture was warmed to rt and stirred for 16 h. The solution was poured into water (20 mL) and extracted with ethyl acetate (3×20 mL). The combined organic layers were washed with saturated brine (20 mL), dried over anhydrous sodium sulfate, filtered, and concentrated under reduced pressure. The resulting residue was purified by silica gel flash chromatography to provide the desired product (94.3 mg, 26% yield) as a light-yellow solid. MS (ESI) m/z=214.0 [M+H]⁺.

The remaining steps were performed according to the standard procedures for preparing GS-456 and GS-438 to provide the desired product (2.1 mg, 4% yield over 3 steps) as a light-yellow solid. MS (ESI) m/z=581.4 [M+H]⁺.

### Example 430. 5-(((trans-3-(3-Cyclopropyl-4-(1-methylpiperidin-3-yl)-1H-pyrazol-1-yl)cyclobutyl)methyl)amino)-2-(2,6-dioxopiperidin-3-yl)isoindoline-1,3-dione (GS-472)

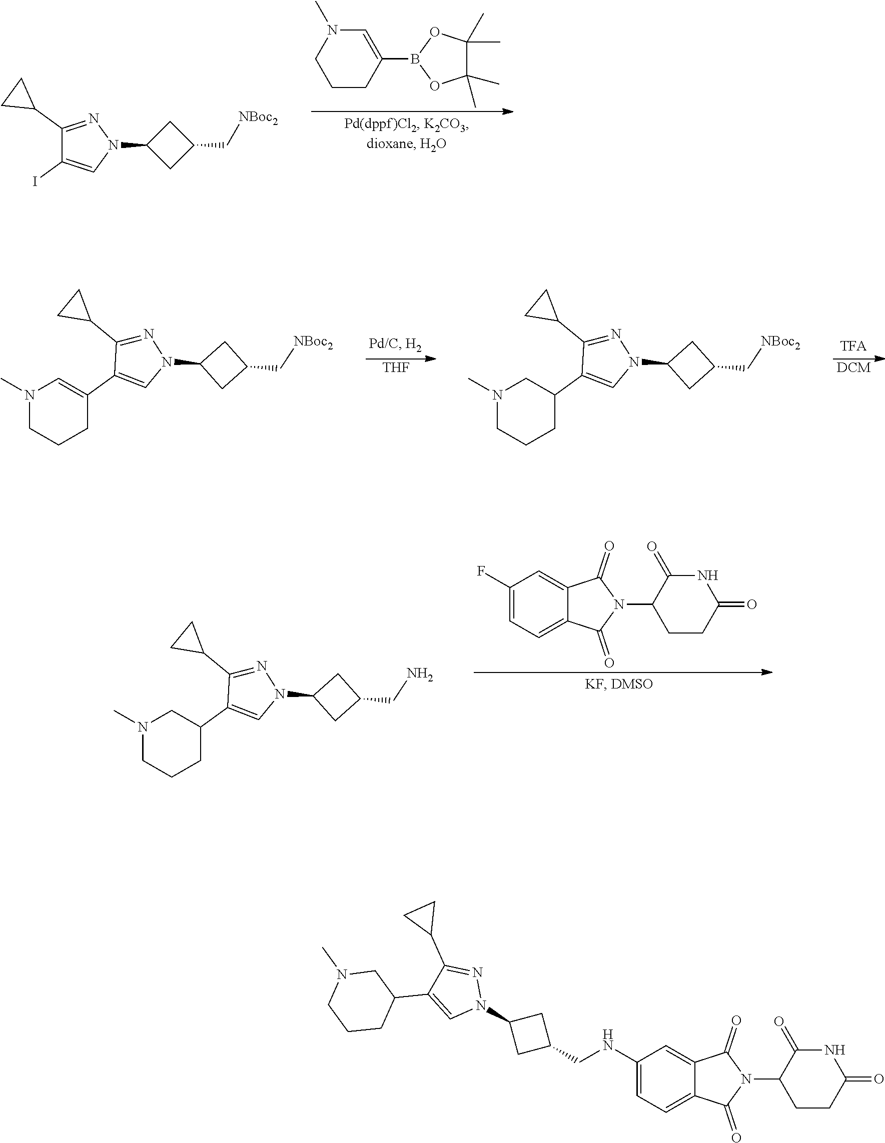

GS-472 was synthesized following the standard procedures for preparing GS-460 (1.1 mg, 9% yield over 4 steps) as a light-yellow solid. MS (ESI) m/z=545.4 [M+H]⁺.

### Example 431. 5-(((trans-3-(3-Cyclopropyl-1H-pyrazolo[3,4-c]pyridin-1-yl)cyclobutyl)methyl) amino)-2-(2,6-dioxopiperidin-3-yl)isoindoline-1,3-dione (GS-473)

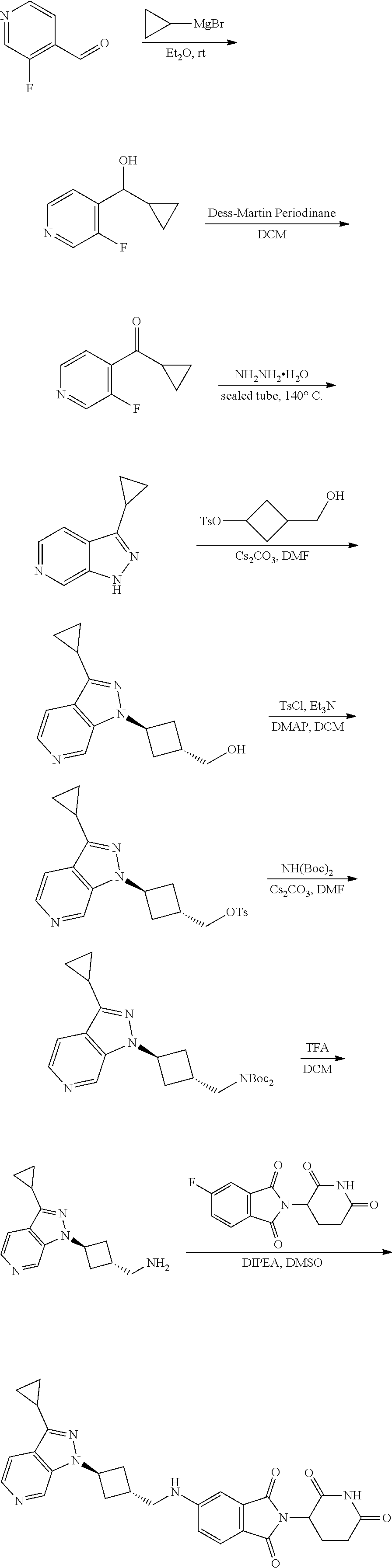

GS-473 was synthesized following the standard procedures for preparing GS-442 (1.2 mg, 1% yield over 8 steps) as a yellow solid. MS (ESI) m/z=499.3 [M+H]⁺.

### Example 432. 5-(((trans-3-(3-Cyclopropyl-1H-pyrazolo[4,3-c]pyridin-1-yl)cyclobutyl)methyl)amino)-2-(2,6-dioxopiperidin-3-yl)isoindoline-1,3-dione (GS-474)

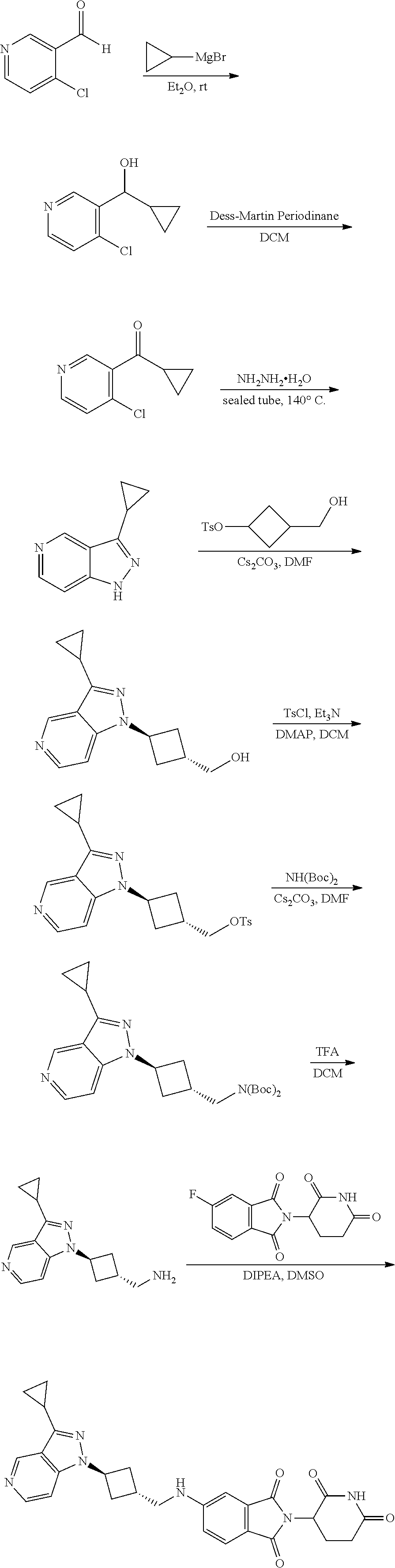

GS-474 was synthesized following the standard procedures for preparing GS-442 (5.3 mg, 2% yield over 8 steps) as a light-yellow solid. MS (ESI) m/z=499.4 [M+H]⁺.

### Example 433. 5-(((trans-3-(3-Cyclopropyl-1H-pyrazolo[4,3-b]pyridin-1-yl)cyclobutyl)methyl)amino)-2-(2,6-dioxopiperidin-3-yl)isoindoline-1,3-dione (GS-475)

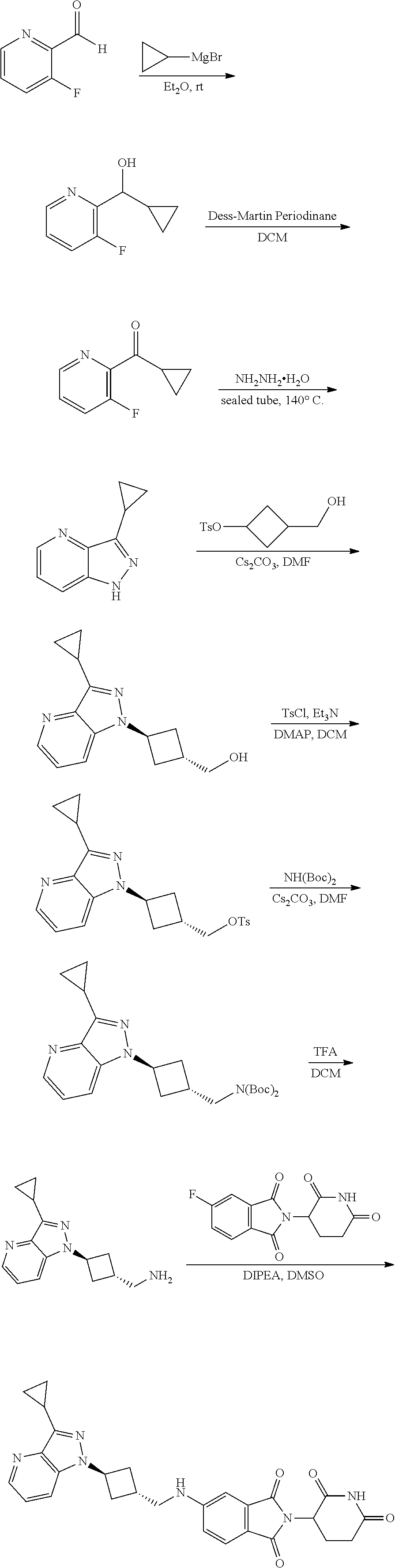

GS-475 was synthesized following the standard procedures for preparing GS-442 (1.5 mg, 1% yield over 8 steps) as a light-yellow solid. MS (ESI) m/z=499.3 [M+H]⁺.

### Example 434. N-((2-(2,6-Dioxopiperidin-3-yl)-1-oxoisoindolin-5-yl)methyl)-3-(4-(4-(quinoxalin-2-yl)-1H-pyrazol-1-yl)piperidin-1-yl)benzamide (GS-476)

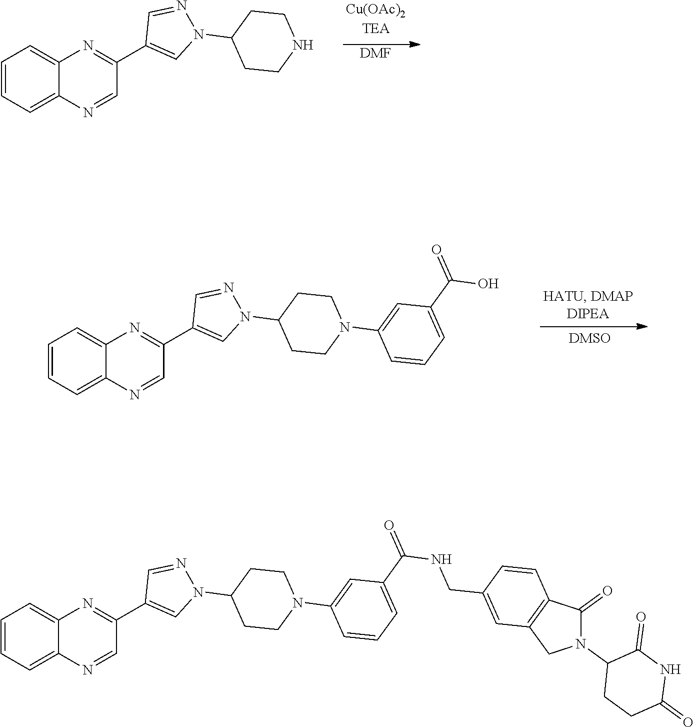

Step 1. Synthesis of 3-(4-(4-(quinoxalin-2-yl)-1H-pyrazol-1-yl)piperidin-1-yl)benzoic acid

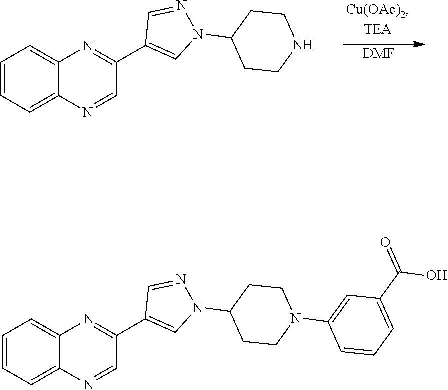

To a mixture of 2-(1-(piperidin-4-yl)-1H-pyrazol-4-yl)quinoxaline (20 mg, 71 μmol) and 3-boronobenzoic acid (24 mg, 143 μmol) in DMF (0.5 mL) were added Cu(OAc)₂ (26 mg, 143 μmol) and TEA (21 mg, 215 μmol). The reaction mixture was purged with N₂ three times and stirred at 50° C. overnight. The reaction was purified by reverse phase chromatography (0-70% MeCN in H₂O) to provide the title compound (15 mg, yield: 52%) as light solid. MS (ESI) m/z=400.4 [M+H]⁺.

Step 2. Synthesis of N-((2-(2,6-dioxopiperidin-3-yl)-1-oxoisoindolin-5-yl)methyl)-3-(4-(4-(quinoxalin-2-yl)-1H-pyrazol-1-yl)piperidin-1-yl)benzamide

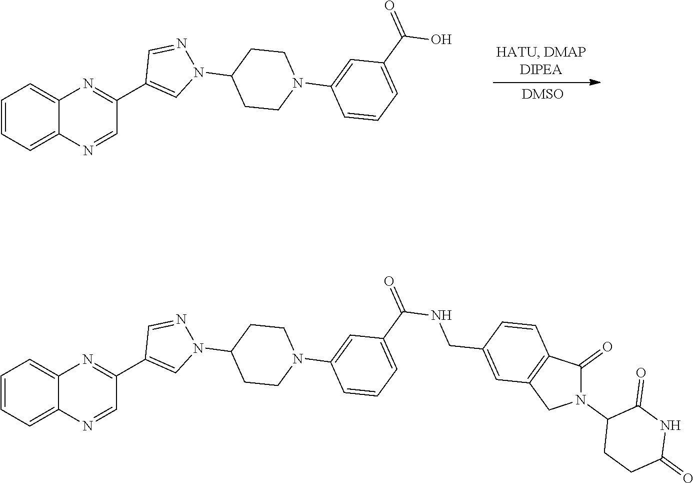 [SMILES: O=C(O)c1cccc(N2CCC(n3cc(-c4cnc5ccccc5n4)cn3)CC2)c1 ; O=C1CCC(N2Cc3cc(CNC(=O)c4cccc(N5CCC(n6cc(-c7cnc8ccccc8n7)cn6)CC5)c4)ccc3C2=O)C(=O)N1]

To a mixture of 3-(4-(4-(quinoxalin-2-yl)-1H-pyrazol-1-yl)piperidin-1-yl)benzoic acid (15 mg, 37 μmol) and 3-(5-(aminomethyl)-1-oxoisoindolin-2-yl)piperidine-2,6-dione (15 mg, 56 μmol) in DMSO (0.5 mL) was added HATU (28 mg, 75 μmol), DMAP (458 ug, 3.7 μmol) and DIPEA (15 mg, 112 μmol). The reaction mixture was stirred at rt overnight. The reaction was purified by reverse phase chromatography (0-70% MeCN in H₂O) to provide the title compound (4.3 mg, yield: 17%) as white solid. MS (ESI) m/z=655.6 [M+H]⁺.

### Example 435. 2-(2,6-Dioxopiperidin-3-yl)-5-((6-oxo-6-(4-(quinoxalin-2-yl)piperidin-1-yl)hexyl)amino)isoindoline-1,3-dione (GS-477)

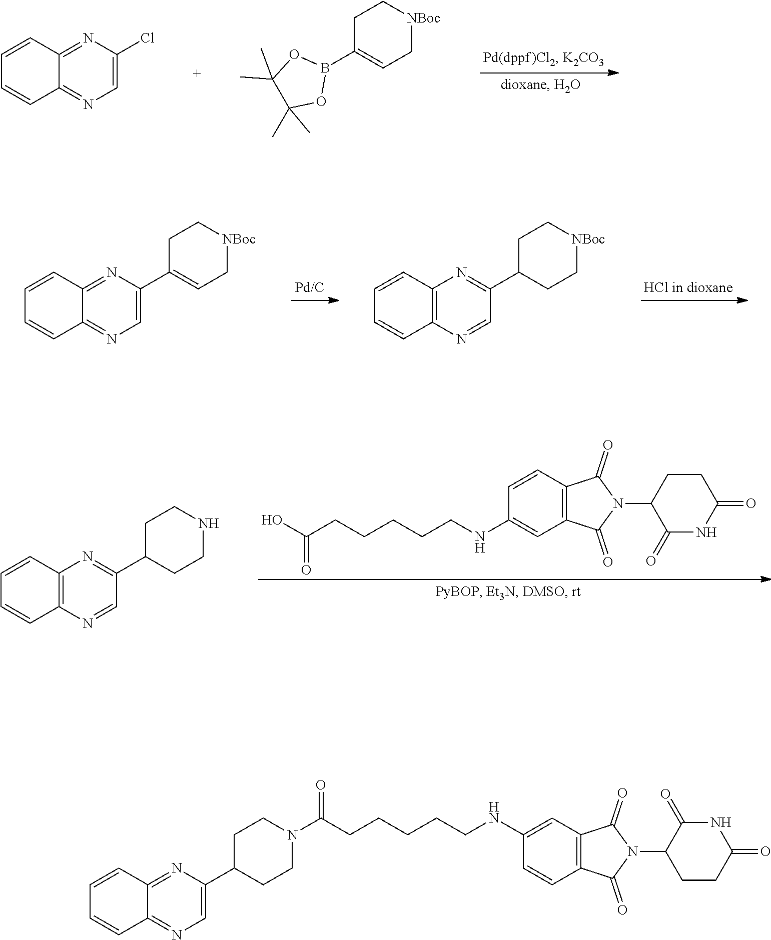

GS-477 was synthesized following the similar procedures as described for GS-043 (8.3 mg). MS (ESI) m/z=583.6 [M+H]⁺.

### Example 436. 2-(2,6-Dioxopiperidin-3-yl)-5-((6-oxo-6-(4-(quinolin-2-yl)-3,6-dihydropyridin-1(2H)-yl)hexyl)amino)isoindoline-1,3-dione (GS-478)

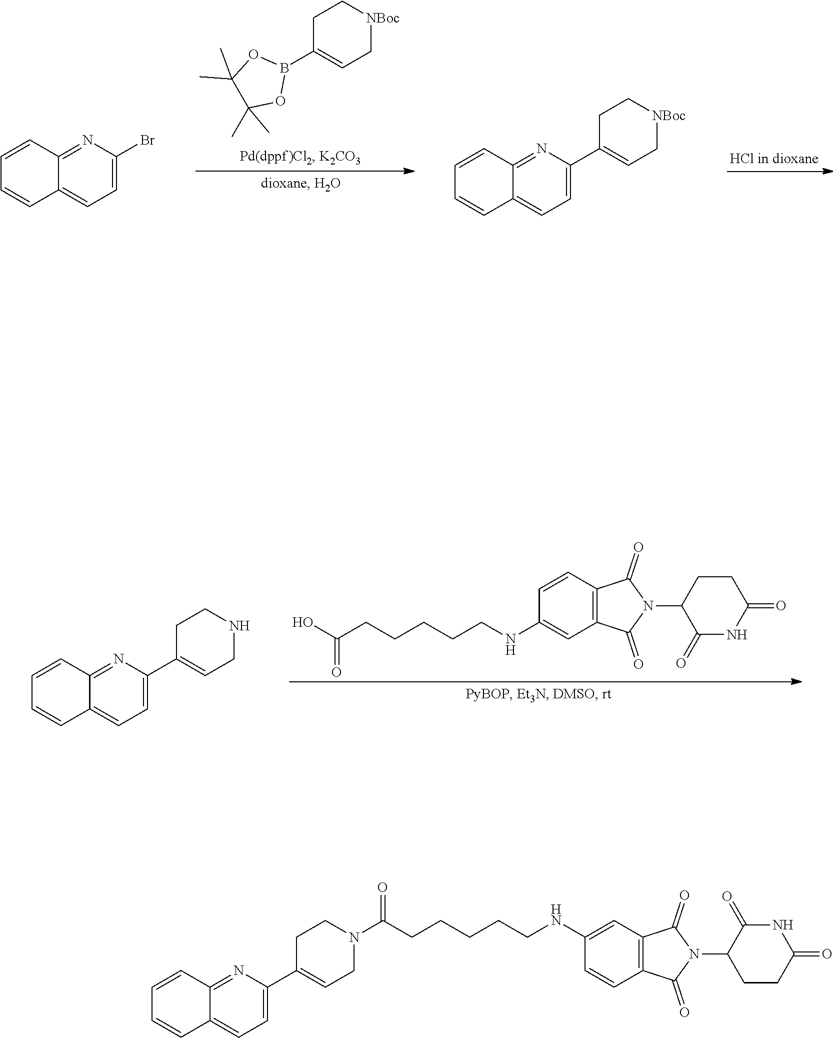 [SMILES: Brc1ccc2ccccc2n1 ; CC(C)(C)OC(=O)N1CC=C(B2OC(C)(C)C(C)(C)O2)CC1 ; CC(C)(C)OC(=O)N1CC=C(c2ccc3ccccc3n2)CC1 ; C1=C(c2ccc3ccccc3n2)CCNC1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CC=C(c5ccc6ccccc6n5)CC4)cc3C2=O)C(=O)N1]

GS-478 was synthesized following the similar procedures as described for GS-043 (10.2 mg). MS (ESI) m/z=580.6 [M+H]⁺.

### Example 437. 2-(2,6-Dioxopiperidin-3-yl)-5-((6-oxo-6-(4-(quinolin-2-yl)piperidin-1-yl)hexyl)amino)isoindoline-1,3-dione (GS-479)

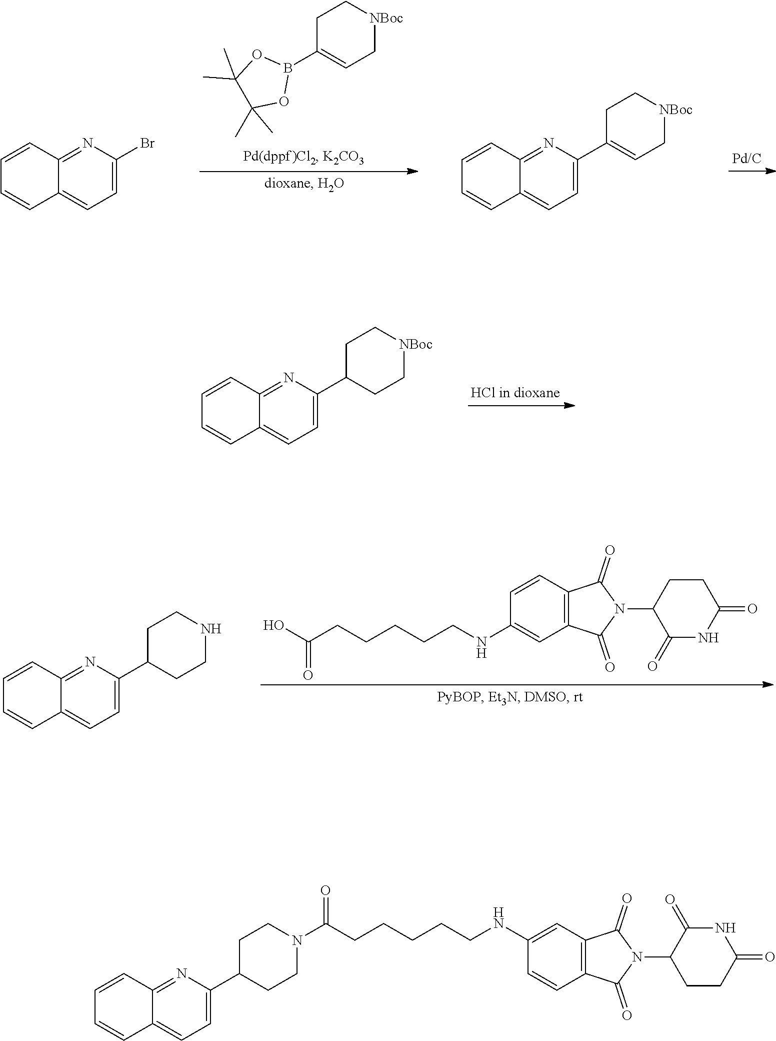

GS-479 was synthesized following the similar procedures as described for GS-043 (8.3 mg). MS (ESI) m/z=582.5 [M+H]⁺.

### Example 438. 2-(2,6-Dioxopiperidin-3-yl)-5-((6-oxo-6-(4-(4-(8-(1,2,3,6-tetrahydropyridin-4-yl)quinoxalin-2-yl)-1H-pyrazol-1-yl)piperidin-1-yl)hexyl)amino)isoindoline-1,3-dione (GS-480)

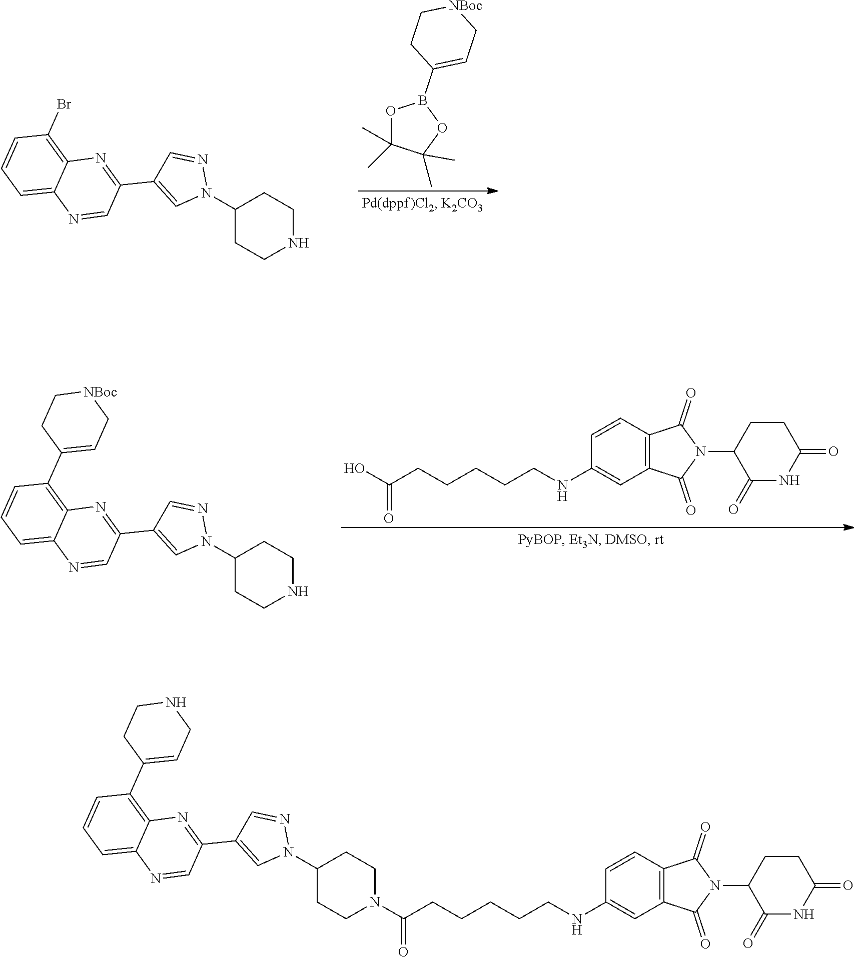 [SMILES: Brc1cccc2ncc(-c3cnn(C4CCNCC4)c3)nc12 ; CC(C)(C)OC(=O)N1CC=C(B2OC(C)(C)C(C)(C)O2)CC1 ; CC(C)(C)OC(=O)N1CC=C(c2cccc3ncc(-c4cnn(C5CCNCC5)c4)nc23)CC1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(n5cc(-c6cnc7cccc(C8=CCNCC8)c7n6)cn5)CC4)cc3C2=O)C(=O)N1]

GS-480 was synthesized following the similar procedures as described for GS-043 (2.3 mg). MS (ESI) m/z=730.8 [M+H]⁺.

### Example 439. 2-(2,6-Dioxopiperidin-3-yl)-5-((6-oxo-6-(4-(quinolin-3-yl)piperidin-1-yl)hexyl)amino)isoindoline-1,3-dione (GS-481) PGP-1607 C₃

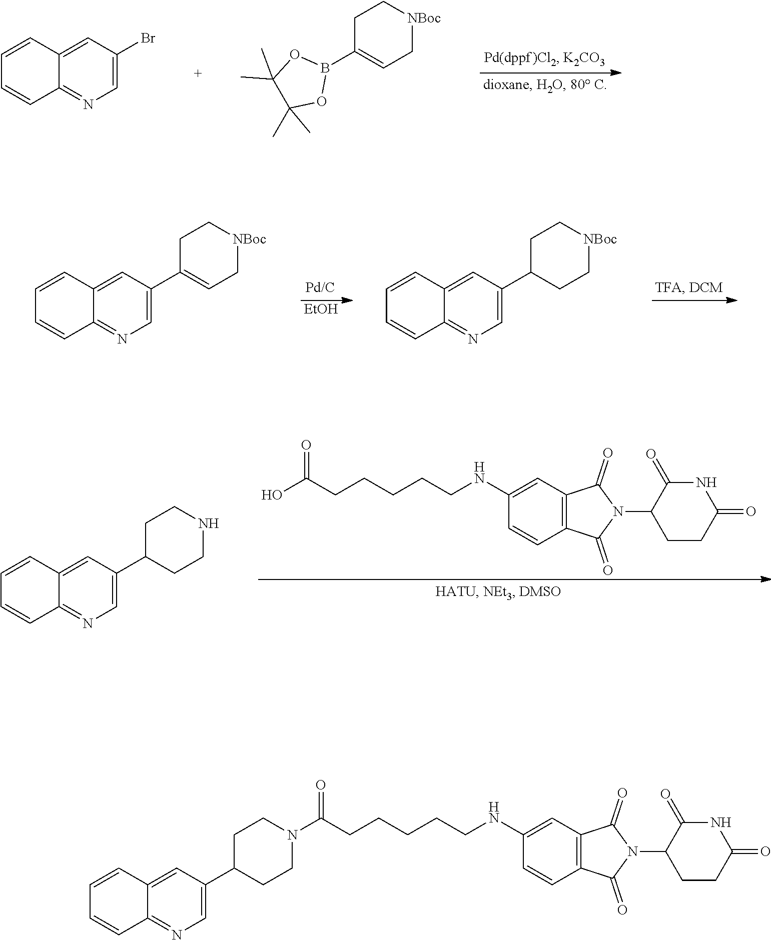

Step 1 to 3. Synthesis of 3-(piperidin-4-yl)quinoline

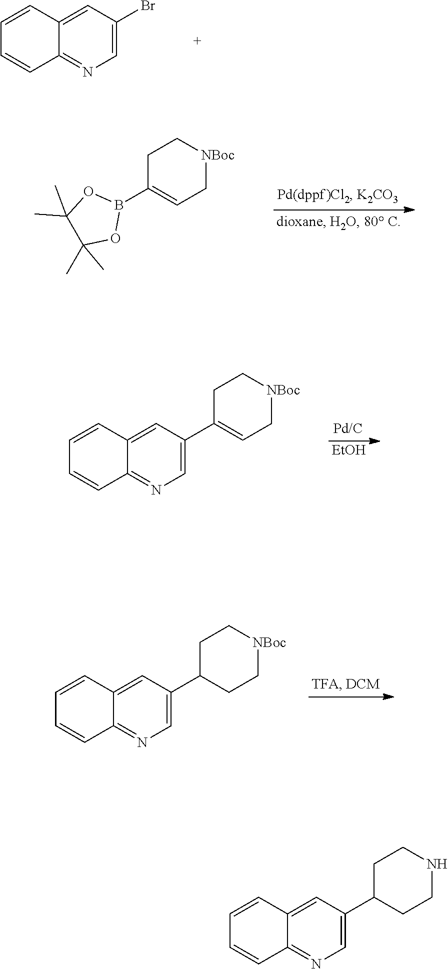

The title compound was synthesized following the similar procedures as described for GS-043 (35 mg, yield over 3 steps: 1%). MS (ESI) m/z=582.5 [M+H]⁺.

Step 4. Synthesis of 2-(2,6-dioxopiperidin-3-yl)-5-((6-oxo-6-(4-(quinolin-3-yl)piperidin-1-yl)hexyl)amino)isoindoline-1,3-dione

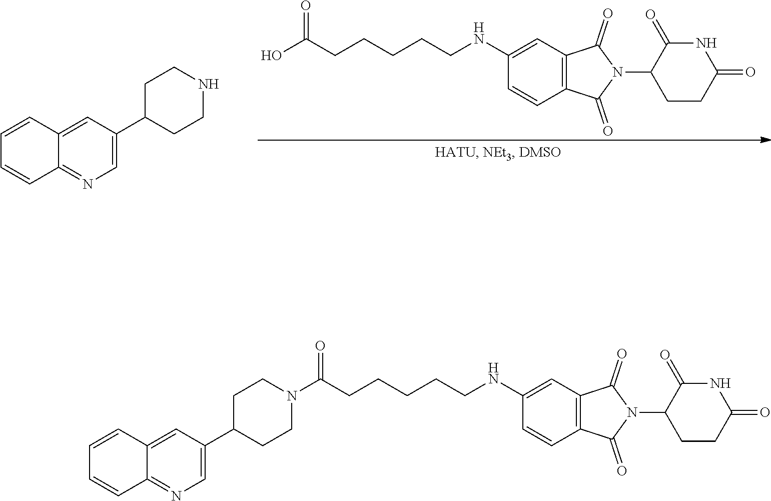 [SMILES: c1ccc2ncc(C3CCNCC3)cc2c1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(c5cnc6ccccc6c5)CC4)cc3C2=O)C(=O)N1]

A solution of 3-(piperidin-4-yl)quinoline (35 mg, 164.87 μmol), 6-((2-(2,6-dioxopiperidin-3-yl)-1,3-dioxoisoindolin-5-yl)amino)hexanoic acid (76.64 mg, 197.84 μmol), HATU (125.38 mg, 329.74 μmol), DIPEA (63.92 mg, 494.61 μmol) in DMSO (1 mL) was stirred at rt overnight. The mixture was purified by reverse phase chromatography to provide the title compound (18.5 mg, yield: 19%). MS (ESI) m/z=582.5 [M+H]⁺.

### Example 440. 2-(2,6-Dioxopiperidin-3-yl)-5-((6-oxo-6-(4-(thiazol-2-yl)piperidin-1-yl)hexyl)amino)isoindoline-1,3-dione (GS-482)

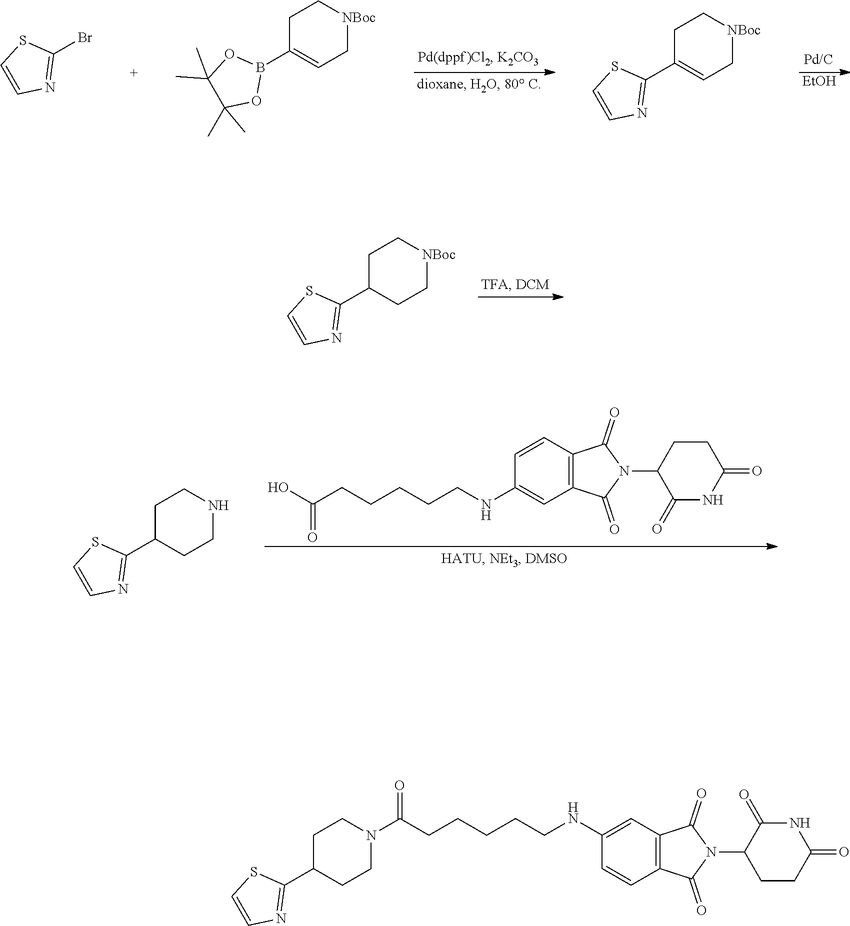

GS-482 was synthesized following the similar procedures as described for GS-481 (15.5 mg, yield over 4 steps: 13%). MS (ESI) m/z=538.4 [M+H]⁺.

### Example 441. N-((2-(2,6-Dioxopiperidin-3-yl)-1-oxoisoindolin-5-yl)methyl)-2-(3-(4-(pyridin-2-yl)piperazin-1-yl)phenyl)acetamide (GS-483)

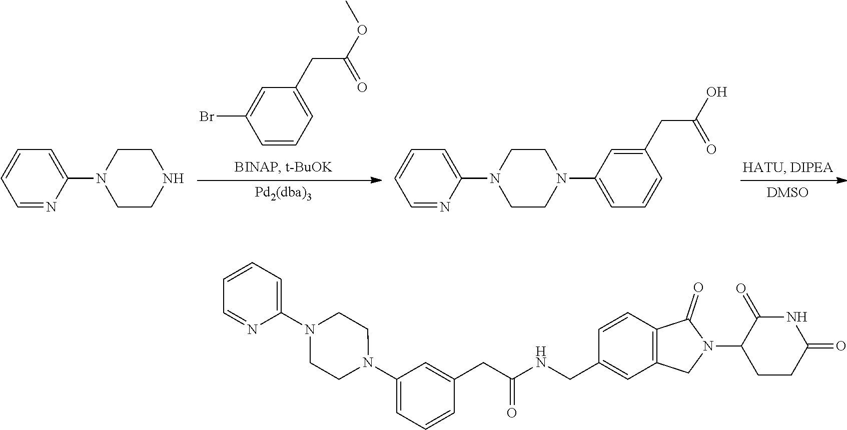 [SMILES: c1ccc(N2CCNCC2)nc1 ; COC(=O)Cc1cccc(Br)c1 ; O=C(O)Cc1cccc(N2CCN(c3ccccn3)CC2)c1 ; O=C(Cc1cccc(N2CCN(c3ccccn3)CC2)c1)NCc1ccc2c(c1)CN(C1CCC(=O)NC1=O)C2=O]

Step 1. Synthesis of 2-(3-(4-(pyridin-2-yl)piperazin-1-yl)phenyl)acetic acid

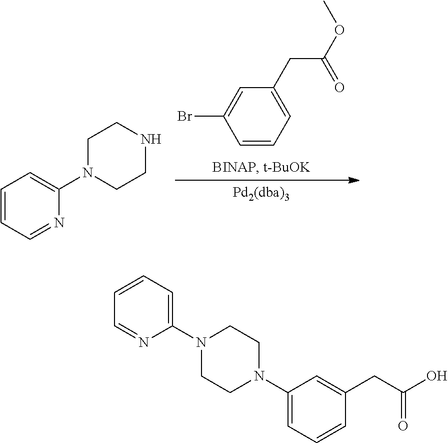 [SMILES: c1ccc(N2CCNCC2)nc1 ; COC(=O)Cc1cccc(Br)c1 ; O=C(O)Cc1cccc(N2CCN(c3ccccn3)CC2)c1]

To a mixture of 1-(2-pyridyl)piperazine (1.0 g, 6.1 mmol) and methyl 2-(4-bromophenyl)acetate (1.68 g, 7.35 mmol) in toluene (15 mL) were added BINAP (3.8 g, 6.1 mmol), t-BuOK (686 mg, 6.1 mmol) and Pd₂(dba)₃ (3.5 g, 6.1 mmol). The reaction mixture was purged with N₂ for three times. The mixture was stirred at 100° C. for 4 h. The solution was filtered, and concentrated. The resulting residue was purified by reverse phase chromatography (0-70% MeCN in H₂O) to provide the title compound (130 mg, yield: 7%) as brown oil. MS (ESI) m/z=298.1 [M+H]⁺.

Step 2. Synthesis of N-((2-(2,6-dioxopiperidin-3-yl)-1-oxoisoindolin-5-yl)methyl)-2-(3-(4-(pyridin-2-yl)piperazin-1-yl)phenyl)acetamide

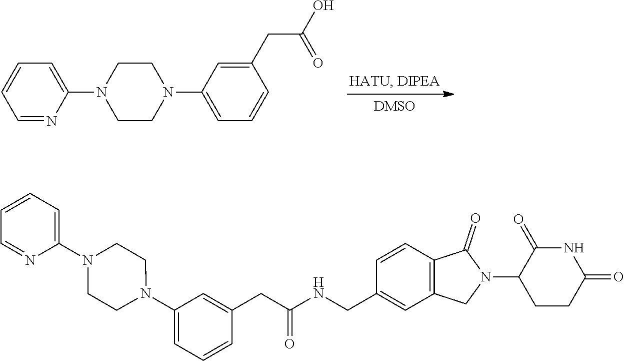 [SMILES: O=C(O)Cc1cccc(N2CCN(c3ccccn3)CC2)c1 ; O=C(Cc1cccc(N2CCN(c3ccccn3)CC2)c1)NCc1ccc2c(c1)CN(C1CCC(=O)NC1=O)C2=O]

To a mixture of 2-(3-(4-(pyridin-2-yl)piperazin-1-yl)phenyl)acetic acid (10 mg, 33 μmol) and 3-[5-(aminomethyl)-1-oxo-isoindolin-2-yl]piperidine-2,6-dione (14 mg, 50 μmol) in DMSO (0.5 mL) were added HATU (12 mg, 33 μmol) and DIPEA (4 mg, 33 μmol). The reaction mixture was stirred at rt overnight. The reaction was purified by reverse phase chromatography (0-70% MeCN in H₂O) to provide the title compound (4.2 mg, yield: 19%) as white solid. MS (ESI) m/z=553.6 [M+H]⁺.

### Example 442. 3-(1-Oxo-5-((5-oxo-5-(4-(4-(quinoxalin-2-yl)-1H-pyrazol-1-yl)piperidin-1-yl)pentyl)oxy)isoindolin-2-yl)piperidine-2,6-dione (GS-484)

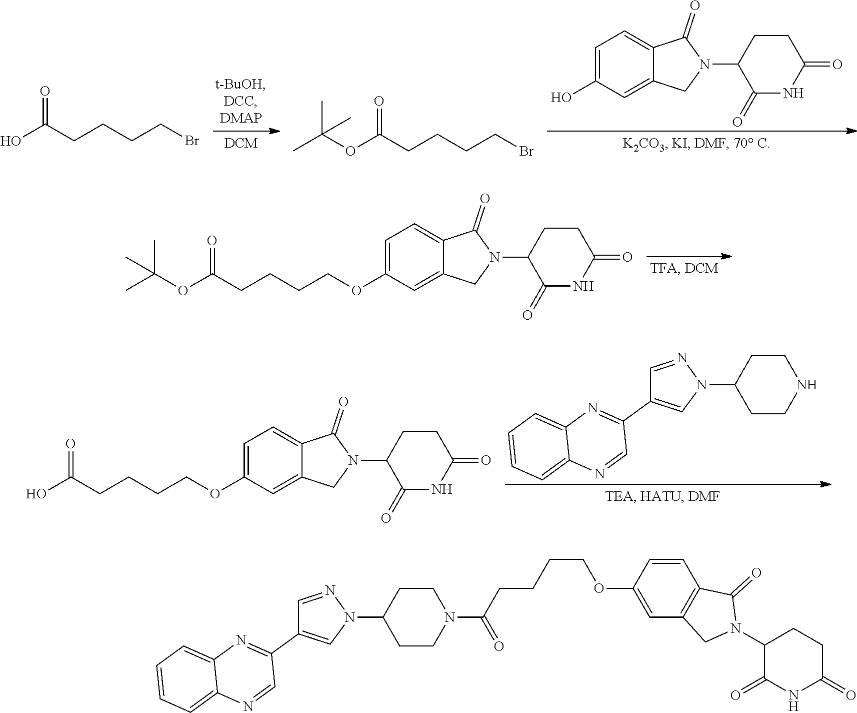

Step 1. Synthesis of tert-butyl 5-bromopentanoate

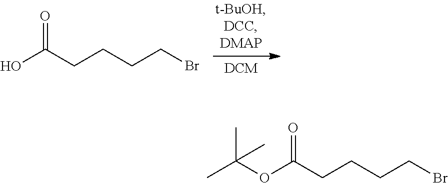

To a solution of 5-bromopentanoic acid (5 g, 27.62 mmol) in DCM (100 mL) were added 2-methylpropan-2-ol (6.14 g, 82.86 mmol), N,N′-dicyclohexylmethanediimine (6.27 g, 30.38 mmol), DMAP (337.43 mg, 2.76 mmol) at 25° C. The mixture was stirred at 25° C. for 18 h, and then filtered through a pad of silica gel. The filtrate was washed with aq. NaOH solution (1 M, 100 mL). The organic layer was concentrated in vacuo to provide the title compound (6.35 g, yield: 97%) as colorless oil.

Step 2. Synthesis of tert-butyl 5-((2-(2,6-dioxopiperidin-3-yl)-1-oxoisoindolin-5-yl)oxy)pentanoate

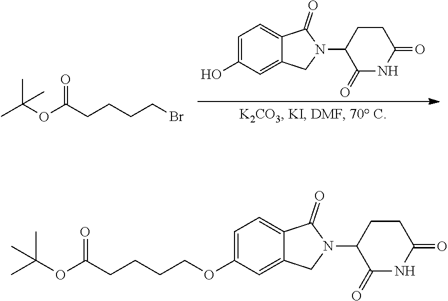 [SMILES: CC(C)(C)OC(=O)CCCCBr ; O=C1CCC(N2Cc3cc(O)ccc3C2=O)C(=O)N1 ; CC(C)(C)OC(=O)CCCCOc1ccc2c(c1)CN(C1CCC(=O)NC1=O)C2=O]

To a mixture of tert-butyl 5-bromopentanoate (0.35 g, 1.48 mmol) and 3-(5-hydroxy-1-oxo-isoindolin-2-yl)piperidine-2,6-dione (0.35 g, 1.34 mmol) in DMF (10 mL) was added K₂CO₃ (611.98 mg, 4.43 mmol) at 25° C. The mixture was stirred at 70° C. for 24 h. After it was cooled to 25° C., the mixture was purified by silica gel chromatography to provide the title compound (0.18 g, yield: 29%) as brown solid. MS (ESI) m/z=361.2 [M+H−56]⁺.

Step 3. Synthesis of 5-((2-(2,6-dioxopiperidin-3-yl)-1-oxoisoindolin-5-yl)oxy)pentanoic acid

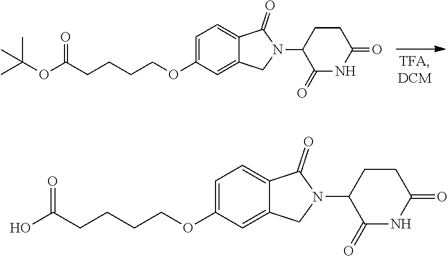 [SMILES: CC(C)(C)OC(=O)CCCCOc1ccc2c(c1)CN(C1CCC(=O)NC1=O)C2=O ; O=C(O)CCCCOc1ccc2c(c1)CN(C1CCC(=O)NC1=O)C2=O]

To a solution of tert-butyl 5-((2-(2,6-dioxopiperidin-3-yl)-1-oxoisoindolin-5-yl)oxy)pentanoate (0.05 g, 120.06 μmol) in DCM (5 mL) was added TFA (684.47 mg, 6.00 mmol) at 25° C. After the reaction was stirred for 18 h. The mixture was concentrated in vacuo to provide the title compound (43 mg, yield: 99%) as brown solid. MS (ESI) m/z=361.2 [M+H]⁺.

Step 4. Synthesis of 3-(1-oxo-5-((5-oxo-5-(4-(4-(quinoxalin-2-yl)-1H-pyrazol-1-yl)piperidin-1-yl)pentyl)oxy)isoindolin-2-yl)piperidine-2,6-dione

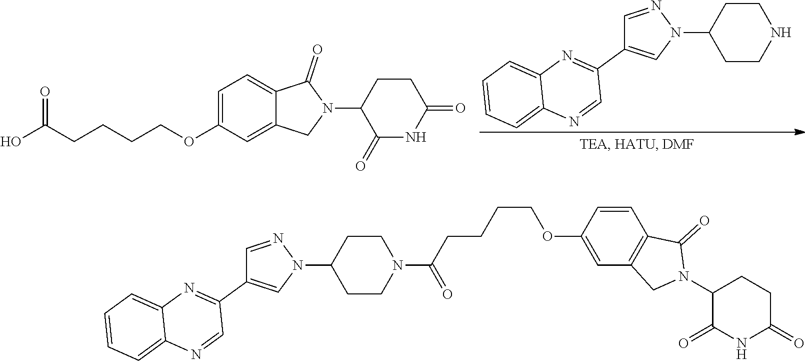

To a solution of 5-((2-(2,6-dioxopiperidin-3-yl)-1-oxoisoindolin-5-yl)oxy)pentanoic acid (43 mg, 119.33 μmol) and 2-(1-(piperidin-4-yl)-1H-pyrazol-4-yl)quinoxaline (see step 2 of GS-005 for synthesis, 33.33 mg, 119.33 μmol) in DMF (3 mL) were added TEA (24.15 mg, 238.65 μmol) and HATU (68.02 mg, 178.99 μmol) at 25° C. The mixture was stirred for 30 min, and purified by silica gel chromatography to provide the title compound (15.5 mg, yield: 21%) as off-white solid. MS (ESI) m/z=622.6 [M+H]⁺.

### Example 443. 3-(1-Oxo-5-(3-oxo-3-(4-(4-(quinoxalin-2-yl)-1H-pyrazol-1-yl)piperidin-1-yl)propoxy)isoindolin-2-yl)piperidine-2,6-dione (GS-485)

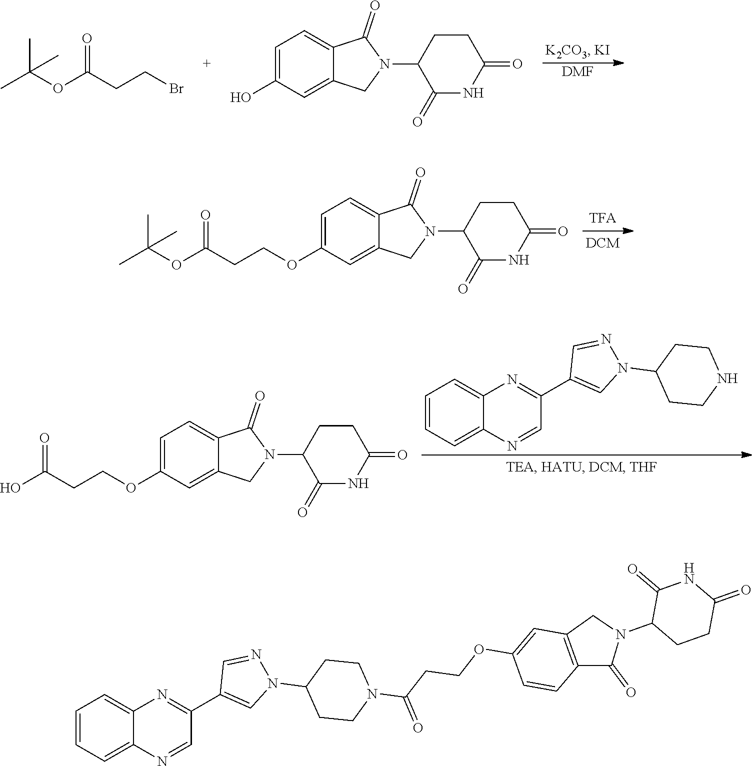

GS-485 was synthesized following the similar procedures as described for GS-484 (17.1 mg) as off-white solid. MS (ESI) m/z=594.5 [M+H]⁺.

### Example 444. 5-((6-(4-(2-Chlorophenyl)piperidin-1-yl)-6-oxohexyl)amino)-2-(2,6-dioxopiperidin-3-yl)isoindoline-1,3-dione (GS-486)

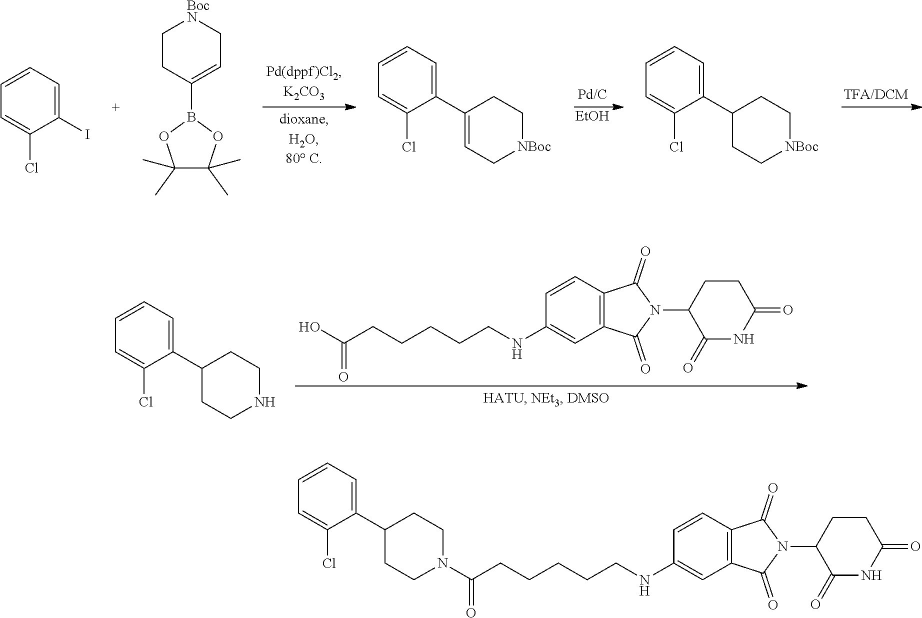

GS-486 was synthesized following the similar procedures as described for GS-481 (1.5 mg, yield over 4 steps: 4%). MS (ESI) m/z=565.4 [M+H]⁺.

### Example 445. 2-(2,6-Dioxopiperidin-3-yl)-5-((6-oxo-6-(4-(o-tolyl)piperidin-1-yl)hexyl)amino)isoindoline-1,3-dione (GS-487)

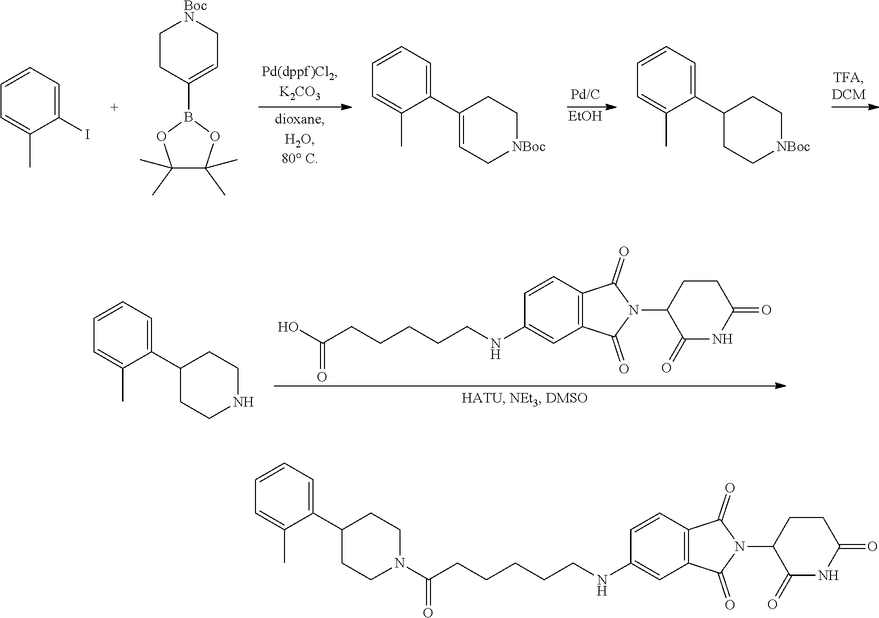

GS-487 was synthesized following the similar procedures as described for GS-481 (18 mg). MS (ESI) m/z=545.4 [M+H]⁺.

### Example 446. 2-(2,6-Dioxopiperidin-3-yl)-5-((6-oxo-6-(4-(m-tolyl)piperidin-1-yl)hexyl)amino)isoindoline-1,3-dione (GS-488)

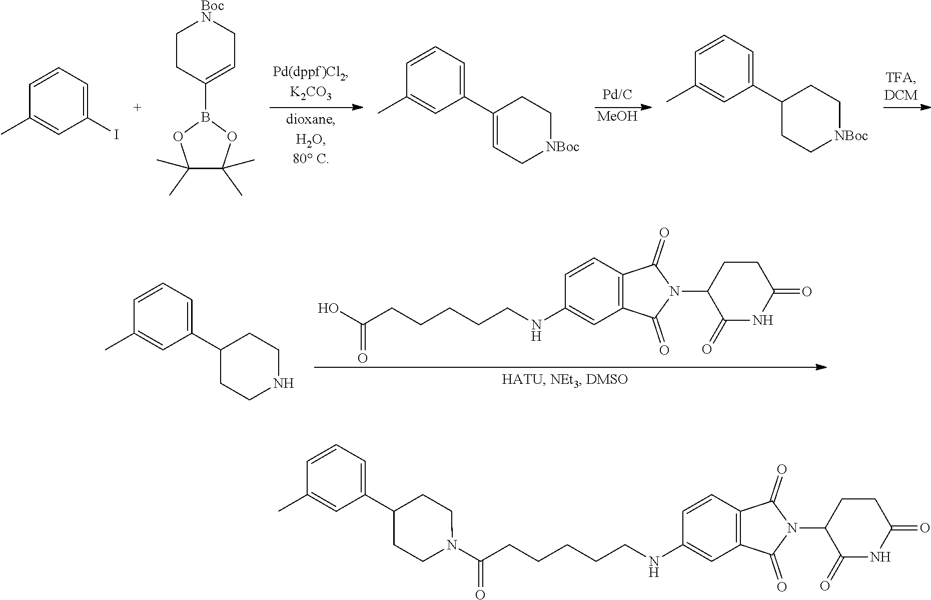

GS-488 was synthesized following the similar procedures as escribed for GS-481 (9.3 mg). MS (ESI) m/z=545.5 [M+H]⁺.

### Example 447. 2-(2,6-Dioxopiperidin-3-yl)-5-((6-oxo-6-(4-(p-tolyl)piperidin-1-yl)hexyl)amino)isoindoline-1,3-dione (GS-489)

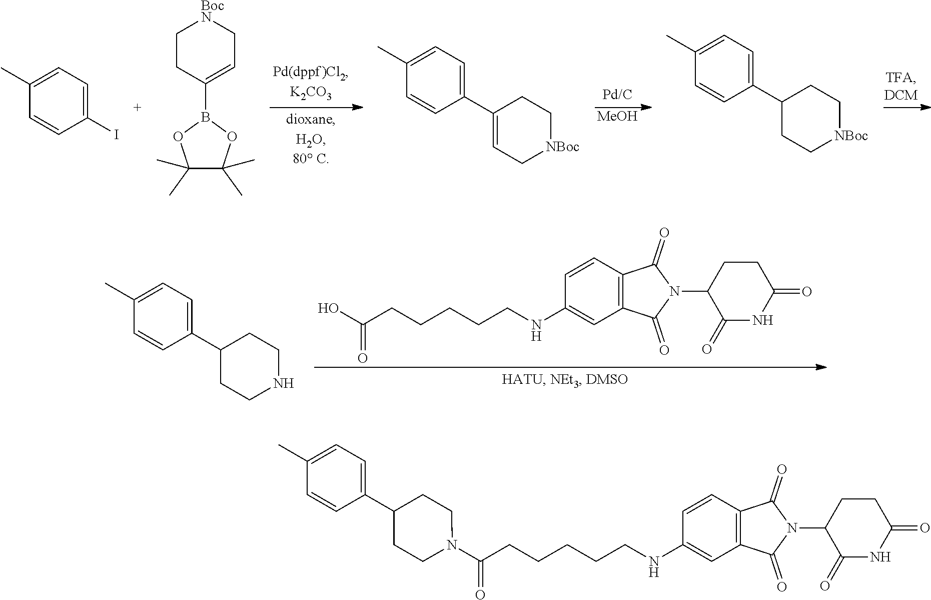

GS-489 was synthesized following the similar procedures as described for GS-481 (7 mg). MS (ESI) m/z=545.5 [M+H]⁺.

### Example 448. 5-((6-(4-(3-Chlorophenyl)piperidin-1-yl)-6-oxohexyl)amino)-2-(2,6-dioxopiperidin-3-yl)isoindoline-1,3-dione (GS-490)

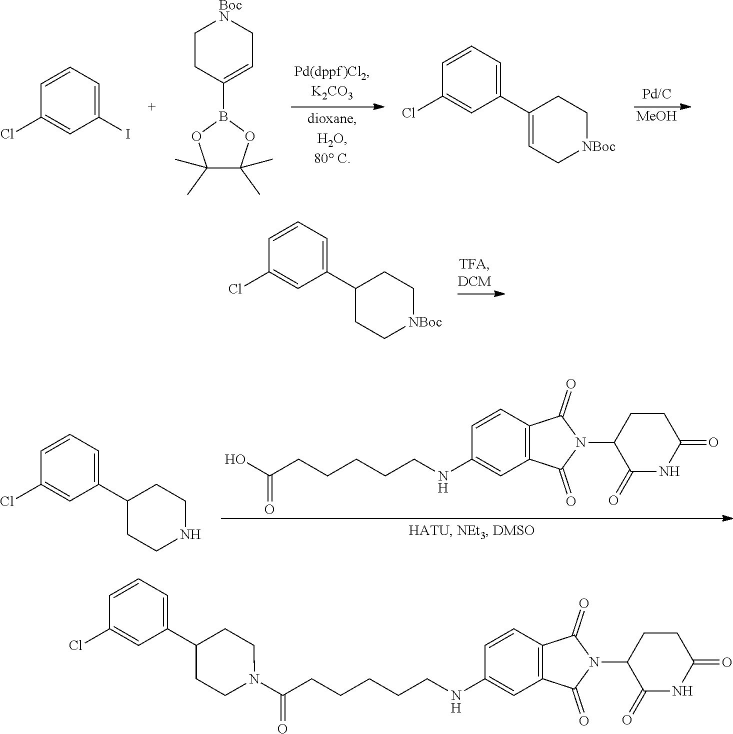

GS-490 was synthesized following the similar procedures as described for GS-481 (6.9 mg). MS (ESI) m/z=565.4 [M+H]⁺.

### Example 449. 5-((6-(4-(4-Chlorophenyl)piperidin-1-yl)-6-oxohexyl)amino)-2-(2,6-dioxopiperidin-3-yl)isoindoline-1,3-dione (GS-491)

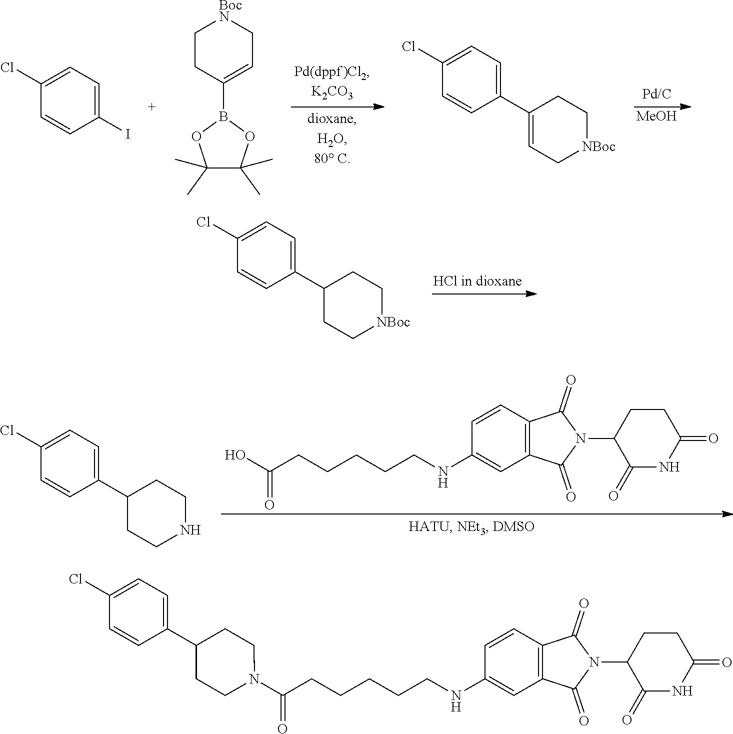

GS-491 was synthesized following the similar procedures as described for GS-486 (10 mg). MS (ESI) m/z=565.4 [M+H]⁺.

### Example 450. 3-(1-Oxo-5-(3-(4-(4-(quinoxalin-2-yl)-1H-pyrazol-1-yl)piperidin-1-yl)propoxy)isoindolin-2-yl)piperidine-2,6-dione (GS-492)

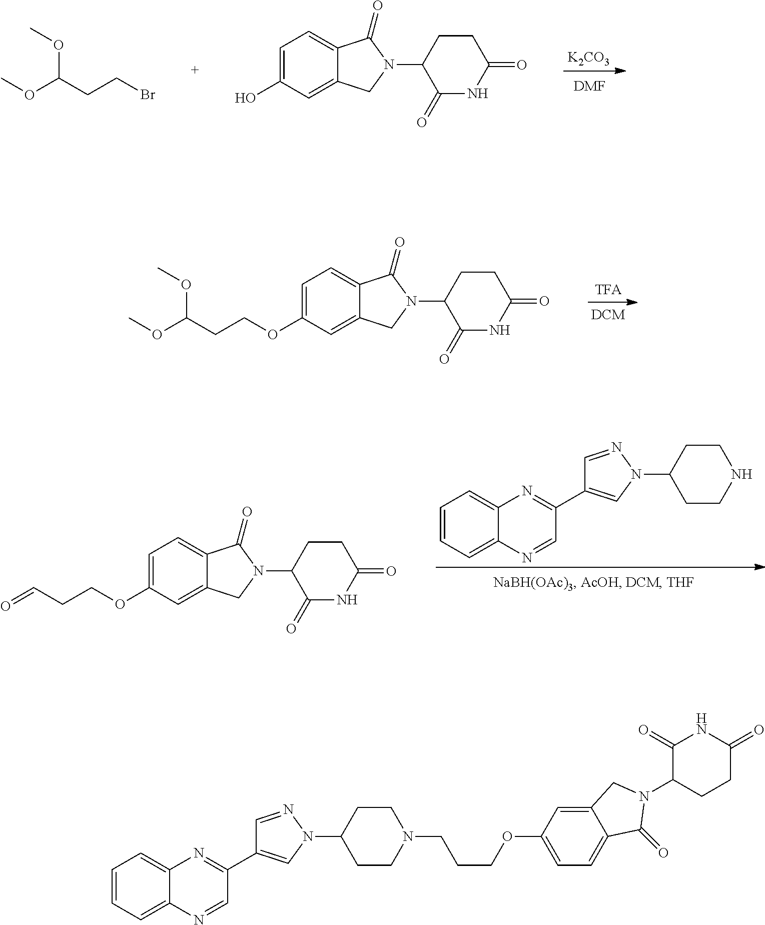

Step 1. Synthesis of 3-(5-(3,3-dimethoxypropoxy)-1-oxoisoindolin-2-yl)piperidine-2,6-dione

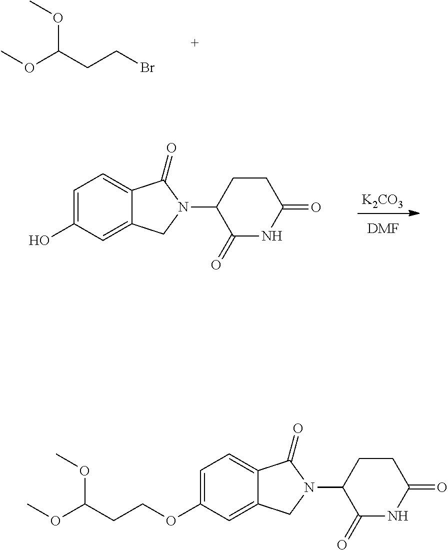

To a mixture of 3-(5-hydroxy-1-oxo-isoindolin-2-yl)piperidine-2,6-dione (350 mg, 1.34 mmol) and 3-bromo-1,1-dimethoxy-propane (295.41 mg, 1.61 mmol) in DMF (6 mL) was added K₂CO₃ (556.78 mg, 4.03 mmol) at 25° C. The mixture was stirred at 70° C. for 18 h. The mixture was cooled to 25° C., and purified by flash chromatography to provide the title compound (80 mg, yield: 16%) as brown solid. MS (ESI) m/z=363.2 [M+H]⁺.

Step 2. Synthesis of 3-((2-(2,6-dioxopiperidin-3-yl)-1-oxoisoindolin-5-yl)oxy)propanal

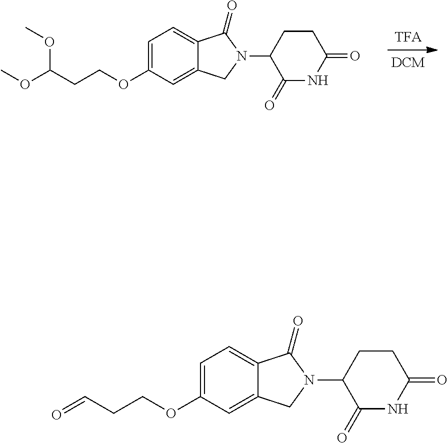 [SMILES: COC(CCOc1ccc2c(c1)CN(C1CCC(=O)NC1=O)C2=O)OC ; O=CCCOc1ccc2c(c1)CN(C1CCC(=O)NC1=O)C2=O]

To a solution of 3-(5-(3,3-dimethoxypropoxy)-1-oxoisoindolin-2-yl)piperidine-2,6-dione (80 mg, 220.77 μmol) in DCM (5 mL) was added TFA (1.26 g, 11.04 mmol) at 25° C. The mixture was stirred for 30 min, and then concentrated in vacuo to provide the title compound (68 mg, yield: 97%) as brown solid. MS (ESI) m/z=317.1 [M+H]⁺.

Step 3. Synthesis of 3-(1-oxo-5-(3-(4-(4-(quinoxalin-2-yl)-1H-pyrazol-1-yl)piperidin-1-yl)propoxy)isoindolin-2-yl)piperidine-2,6-dione

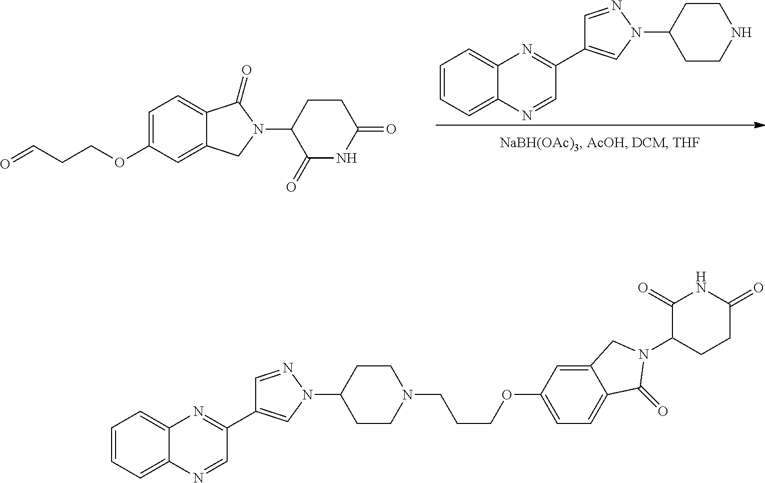 [SMILES: O=CCCOc1ccc2c(c1)CN(C1CCC(=O)NC1=O)C2=O ; c1ccc2nc(-c3cnn(C4CCNCC4)c3)cnc2c1 ; O=C1CCC(N2Cc3cc(OCCCN4CCC(n5cc(-c6cnc7ccccc7n6)cn5)CC4)ccc3C2=O)C(=O)N1]

To a mixture of 3-((2-(2,6-dioxopiperidin-3-yl)-1-oxoisoindolin-5-yl)oxy)propanal (68 mg, 214.98 μmol) and 2-[1-(4-piperidyl)pyrazol-4-yl]quinoxaline (24.02 mg, 85.99 μmol) in THF (3 mL) and DCM (3 mL) was added AcOH (12.91 mg, 214.98 μmol) at 25° C. After the mixture was stirred for 2 h, NaBH(OAc)₃ (136.69 mg, 644.94 μmol) was added. The mixture was stirred for 18 h, before it was quenched with water (1 mL). The obtained mixture was concentrated in vacuo. The resulting residue was purified by flash chromatography, followed by prep-TLC (DCM:MeOH=10:1) to provide the title compound (2 mg, yield: 2%) as white solid. MS (ESI) m/z=580.5 [M+H]⁺.

### Example 451. 3-(1-Oxo-5-((6-oxo-6-(4-(4-(quinoxalin-2-yl)-1H-pyrazol-1-yl)piperidin-1-yl)hexyl)oxy)isoindolin-2-yl)piperidine-2,6-dione (GS-493)

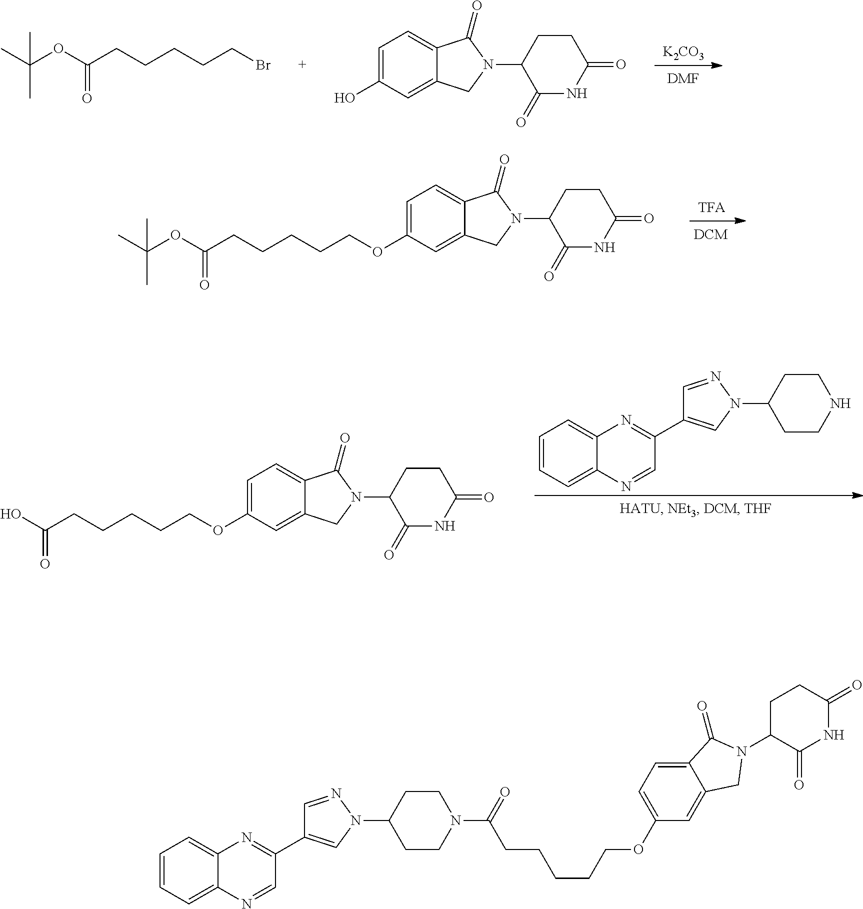

GS-493 was synthesized following the similar procedures as described for GS-484 (17.7 mg) as white solid. MS (ESI) m/z=636.6 [M+H]⁺.

### Example 452. 2-(2,6-Dioxopiperidin-3-yl)-5-((6-oxo-6-(4-(4-(8-(piperidin-4-yl)quinoxalin-2-yl)-1H-pyrazol-1-yl)piperidin-1-yl)hexyl)amino)isoindoline-1,3-dione (GS-044)

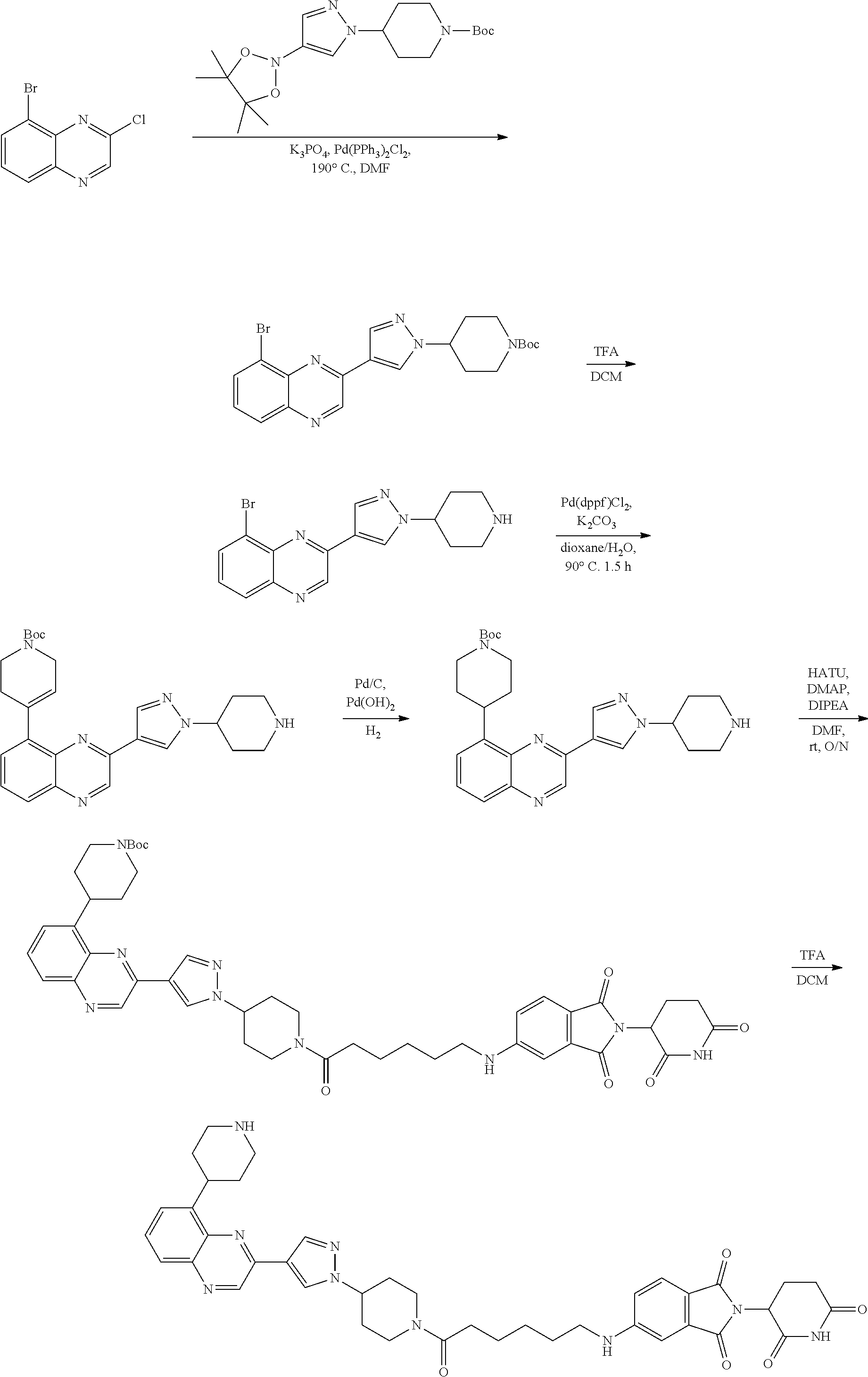

Step 1. Synthesis of tert-butyl 4-(4-(8-bromoquinoxalin-2-yl)-1H-pyrazol-1-yl)piperidine-1-carboxylate

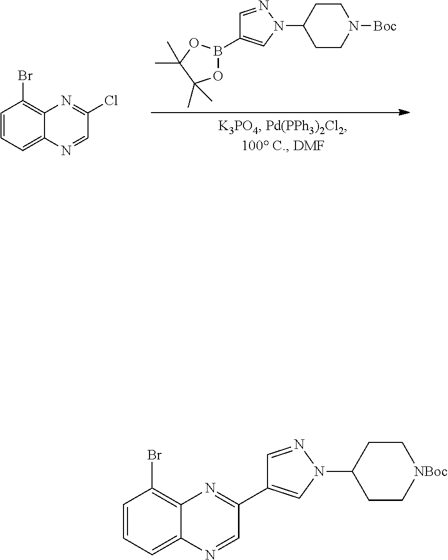 [SMILES: Clc1cnc2cccc(Br)c2n1 ; CC(C)(C)OC(=O)N1CCC(n2cc(B3OC(C)(C)C(C)(C)O3)cn2)CC1 ; CC(C)(C)OC(=O)N1CCC(n2cc(-c3cnc4cccc(Br)c4n3)cn2)CC1]

To a solution of 8-bromo-2-chloroquinoxaline (5.0 g, 20.66 mmol) in DMF (120 mL) were added tert-butyl 4-(4-(4,4,5,5-tetramethyl-1,3,2-dioxaborolan-2-yl)-1H-pyrazol-1-yl)piperidine-1-carboxylate (7.78 g, 20.66 mmol) and K₃PO₄ (13.1 g, 61.48 mmol). The mixture was purged with N₂ for three times, before Pd(PPh₃)₂Cl₂ (435 mg, 0.620 mmol) was added. After the mixture was stirred at 100° C. overnight, it was diluted with H₂O, and then extracted with DCM. The organic phase was washed with brine, dried over Na₂SO₄, filtered, and concentrated. The resulting residue was purified by silica gel chromatography to provide the title compound (6.9 g, yield: 73%) as white solid. ¹H NMR (400 MHz, DMSO-d₆) δ 9.35 (s, 1H), 8.77 (s, 1H), 8.77 (s, 1H), 8.16 (d, J=7.2 Hz, 1H), 8.05 (d, J=7.2 Hz, 1H), 7.65 (t, J=8.0 Hz, 1H), 4.52-4.50 (m, 1H), 4.10-4.07 (m, 2H), 3.05-2.97 (m, 2H), 2.11-2.07 (m, 2H), 1.88-1.84 (m, 2H), 1.43 (s, 9H); MS (ESI) m/z=459.1 [M+H]⁺.

Step 2. Synthesis of 8-bromo-2-(1-(piperidin-4-yl)-1H-pyrazol-4-yl)quinoxaline

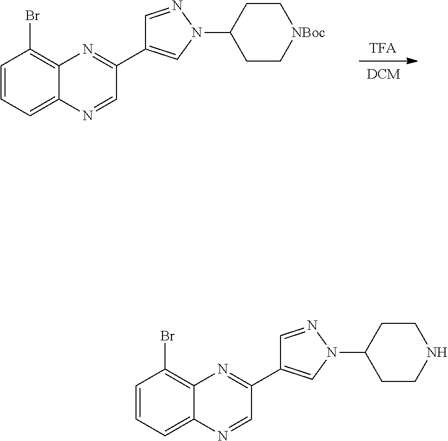 [SMILES: CC(C)(C)OC(=O)N1CCC(n2cc(-c3cnc4cccc(Br)c4n3)cn2)CC1 ; Brc1cccc2ncc(-c3cnn(C4CCNCC4)c3)nc12]

A solution of tert-butyl 4-(4-(8-bromoquinoxalin-2-yl)-1H-pyrazol-1-yl)piperidine-1-carboxylate (1.2 g, 2.62 mmol) in TFA (4 mL) and DCM (6 mL) was stirred at rt for 1 h. The reaction mixture was concentrated to provide the title compound (1.2 g, yield: 97%) as yellow solid. MS (ESI) m/z=358.1 [M+H]⁺.

Step 3. Synthesis of tert-butyl 4-(3-(1-(piperidin-4-yl)-1H-pyrazol-4-yl)quinoxalin-5-yl)-3,6-dihydropyridine-1(2H)-carboxylate

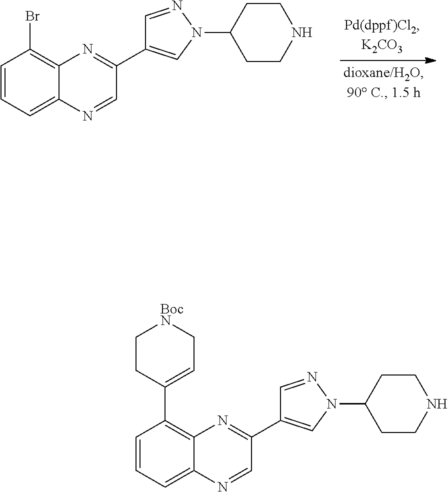 [SMILES: Brc1cccc2ncc(-c3cnn(C4CCNCC4)c3)nc12 ; CC(C)(C)OC(=O)N1CC=C(c2cccc3ncc(-c4cnn(C5CCNCC5)c4)nc23)CC1]

To a mixture of 8-bromo-2-(1-(piperidin-4-yl)-1H-pyrazol-4-yl)quinoxaline (1.2 g, 3.3 mmol) and (1-tert-butoxycarbonyl-3,6-dihydro-2H-pyridin-4-yl)boronic acid (912 mg, 4.0 mmol) in water (4 mL) and dioxane (20 mL) were added K₂CO₃ (462 mg, 3.35 mmol) and Pd(dppf)Cl₂ (245 mg, 0.34 mmol). The reaction mixture was stirred at 90° C. for 1.5 h under N₂. The solution was quenched with water, and extracted with EtOAc. The organic phase was concentrated, and the resulting residue was purified by reverse phase chromatography (0-70% MeCN in H₂O) to provide the title compound (1.1 g, yield: 71%) as brown solid. MS (ESI) m/z=461.4 [M+H]⁺.

Step 4. Synthesis of tert-butyl 4-(3-(1-(piperidin-4-yl)-1H-pyrazol-4-yl)quinoxalin-5-yl)piperidine-1-carboxylate

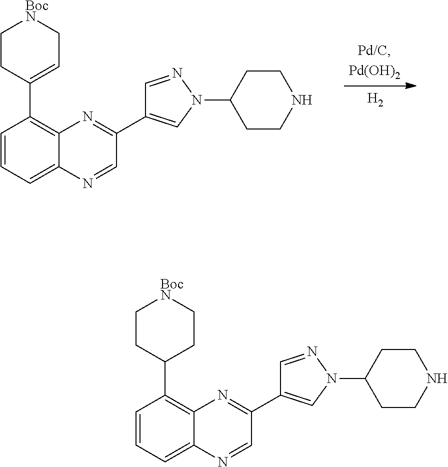 [SMILES: CC(C)(C)OC(=O)N1CC=C(c2cccc3ncc(-c4cnn(C5CCNCC5)c4)nc23)CC1 ; "H2" ; CC(C)(C)OC(=O)N1CCC(c2cccc3ncc(-c4cnn(C5CCNCC5)c4)nc23)CC1]

To a solution of tert-butyl 4-(3-(1-(piperidin-4-yl)-1H-pyrazol-4-yl)quinoxalin-5-yl)-3,6-dihydropyridine-1(2H)-carboxylate (400 mg, 868 μmol) in THF (20 mL) and NH₃H₂O (0.25 mL) was added Pd/C (50 mg, 411 μmol). The reaction mixture was stirred at rt overnight under H₂. After filtration, Pd(OH)₂ (50 mg, 868 μmol) was added to the filtrate. The reaction mixture was stirred at rt overnight under H₂. Then, the mixture was filtered, and the filtrate was concentrated. The residue was purified by reverse phase chromatography (0-70% MeCN in H₂O) to provide the title compound (100 mg, yield: 25%) as white solid. MS (ESI) m/z=463.3 [M+H]⁺.

Step 5. Synthesis of tert-butyl 4-(3-(1-(1-(6-((2-(2,6-dioxopiperidin-3-yl)-1,3-dioxoisoindolin-5-yl)amino)hexanoyl)piperidin-4-yl)-1H-pyrazol-4-yl)quinoxalin-5-yl)piperidine-1-carboxylate

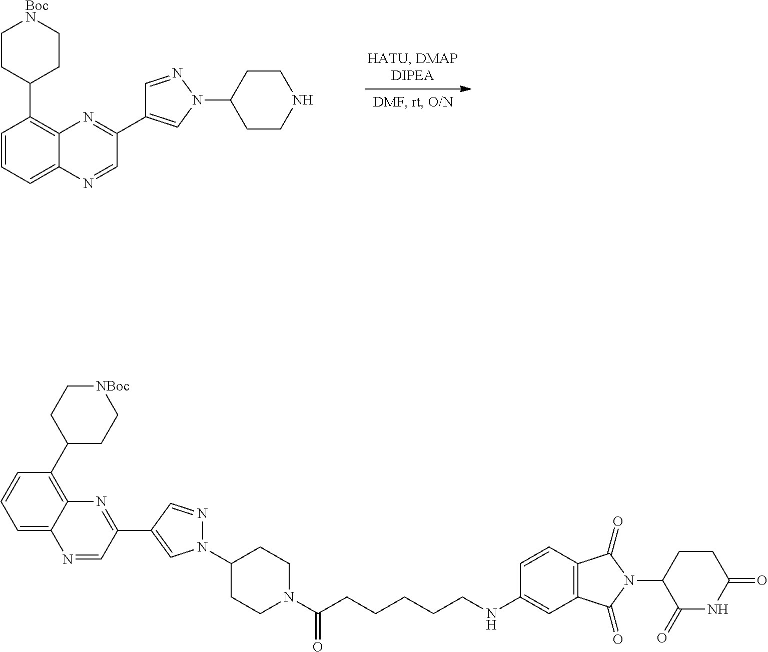 [SMILES: CC(C)(C)OC(=O)N1CCC(c2cccc3ncc(-c4cnn(C5CCNCC5)c4)nc23)CC1 ; CC(C)(C)OC(=O)N1CCC(c2cccc3ncc(-c4cnn(C5CCN(C(=O)CCCCCNc6ccc7c(c6)C(=O)N(C6CCC(=O)NC6=O)C7=O)CC5)c4)nc23)CC1]

To a mixture of tert-butyl 4-(3-(1-(piperidin-4-yl)-1H-pyrazol-4-yl)quinoxalin-5-yl)piperidine-1-carboxylate (100 mg, 216 μmol) and 6-((2-(2,6-dioxopiperidin-3-yl)-1,3-dioxoisoindolin-5-yl)amino)hexanoic acid (92 mg, 237 μmol) in DMF (8 mL) were added HATU (164 mg, 432 μmol), DMAP (2 mg, 21 μmol) and DIPEA (83 mg, 648 μmol). The reaction mixture was stirred at rt overnight. The reaction was purified by reverse phase chromatography (0-70% MeCN in H₂O) to provide the title compound (100 mg, yield: 55%) as yellow solid. MS (ESI) m/z=832.5 [M+H]⁺.

Step 6. Synthesis of 2-(2,6-dioxopiperidin-3-yl)-5-((6-oxo-6-(4-(4-(8-(piperidin-4-yl)quinoxalin-2-yl)-1H-pyrazol-1-yl)piperidin-1-yl)hexyl)amino)isoindoline-1,3-dione

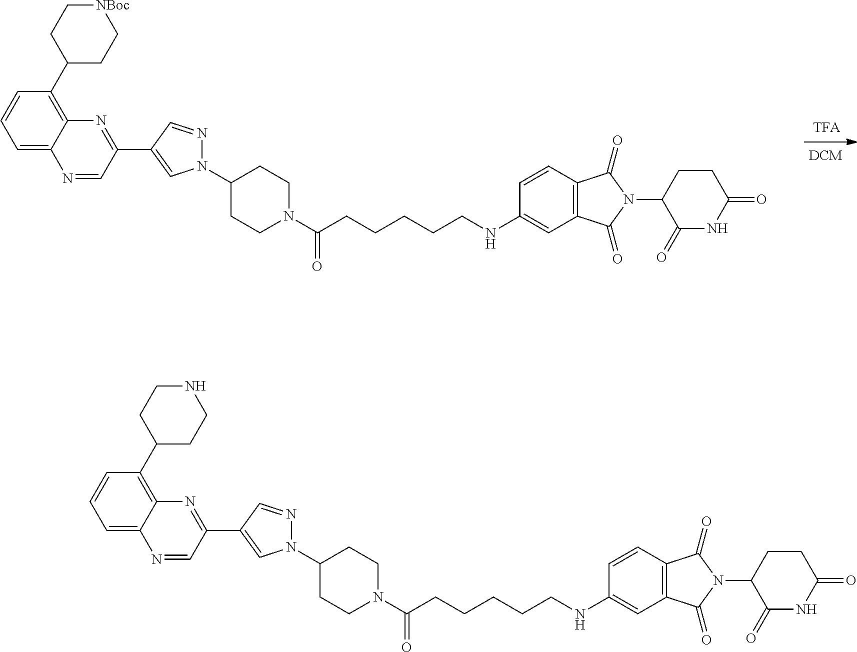 [SMILES: CC(C)(C)OC(=O)N1CCC(c2cccc3ncc(-c4cnn(C5CCN(C(=O)CCCCCNc6ccc7c(c6)C(=O)N(C6CCC(=O)NC6=O)C7=O)CC5)c4)nc23)CC1 ; O=C1CCC(N2C(=O)c3ccc(NCCCCCC(=O)N4CCC(n5cc(-c6cnc7cccc(C8CCNCC8)c7n6)cn5)CC4)cc3C2=O)C(=O)N1]

To a solution of tert-butyl 4-(3-(1-(1-(6-((2-(2,6-dioxopiperidin-3-yl)-1,3-dioxoisoindolin-5-yl)amino)hexanoyl)piperidin-4-yl)-1H-pyrazol-4-yl)quinoxalin-5-yl)piperidine-1-carboxylate (120 mg, 144 μmol) in DCM (4 mL) was added TFA (2 mL). The reaction mixture was stirred at rt for 2 h, and then concentrated. The resulting residue was purified by reverse phase chromatography (0-70% MeCN in H₂O) to provide the title compound (28.9 mg, yield: 27%) as yellow solid. MS (ESI) m/z=732.6 [M+H]⁺.

### Example 453. N-(2-(2,6-Dioxopiperidin-3-yl)-1-oxoisoindolin-5-yl)-2-(3-(4-(4-(quinoxalin-2-yl)-1H-pyrazol-1-yl)piperidin-1-yl)phenyl)acetamide (GS-495)

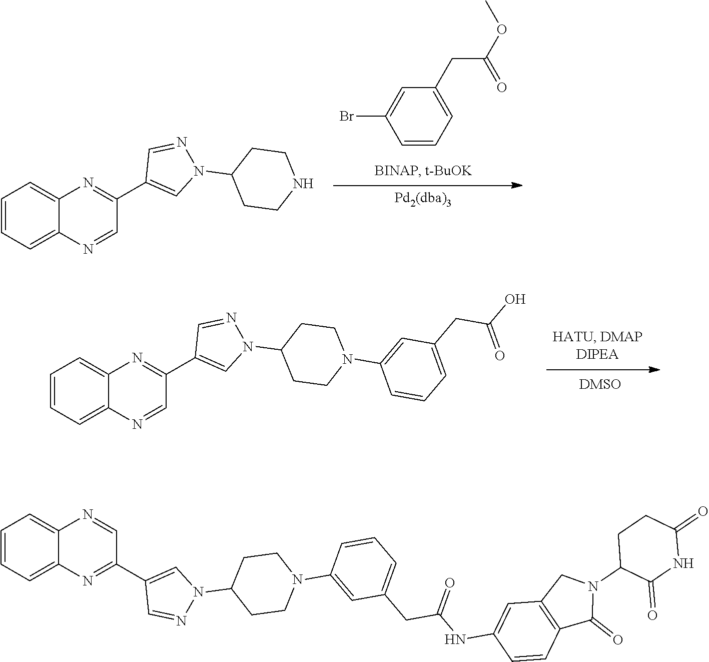 [SMILES: c1ccc2nc(-c3cnn(C4CCNCC4)c3)cnc2c1 ; COC(=O)Cc1cccc(Br)c1 ; O=C(O)Cc1cccc(N2CCC(n3cc(-c4cnc5ccccc5n4)cn3)CC2)c1 ; O=C1CCC(N2Cc3cc(NC(=O)Cc4cccc(N5CCC(n6cc(-c7cnc8ccccc8n7)cn6)CC5)c4)ccc3C2=O)C(=O)N1]

GS-495 was synthesized following the similar procedures as described for GS-483 (1.0 mg) as off-white solid. MS (ESI) m/z=655.6 [M+H]⁺.

### Example 454. 2-(2,6-Dioxopiperidin-3-yl)-5-((6-(4-phenyl-1H-pyrazol-1-yl)hexyl)amino)isoindoline-1,3-dione (GS-496)

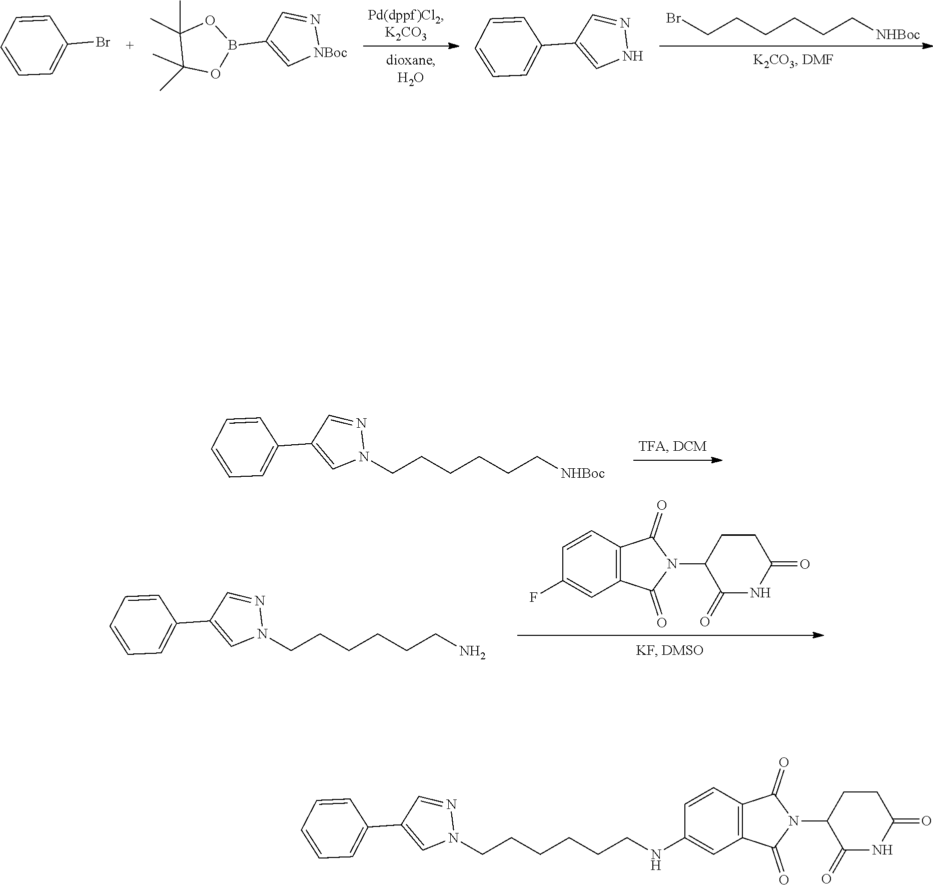

GS-496 was synthesized following the similar procedures as described for GS-498 (3.5 mg). MS (ESI) m/z=500.4 [M+H]⁺.

### Example 455. 2-(2,6-Dioxopiperidin-3-yl)-5-((6-(4-(m-tolyl)-1H-pyrazol-1-yl)hexyl)amino)isoindoline-1,3-dione (GS-497)

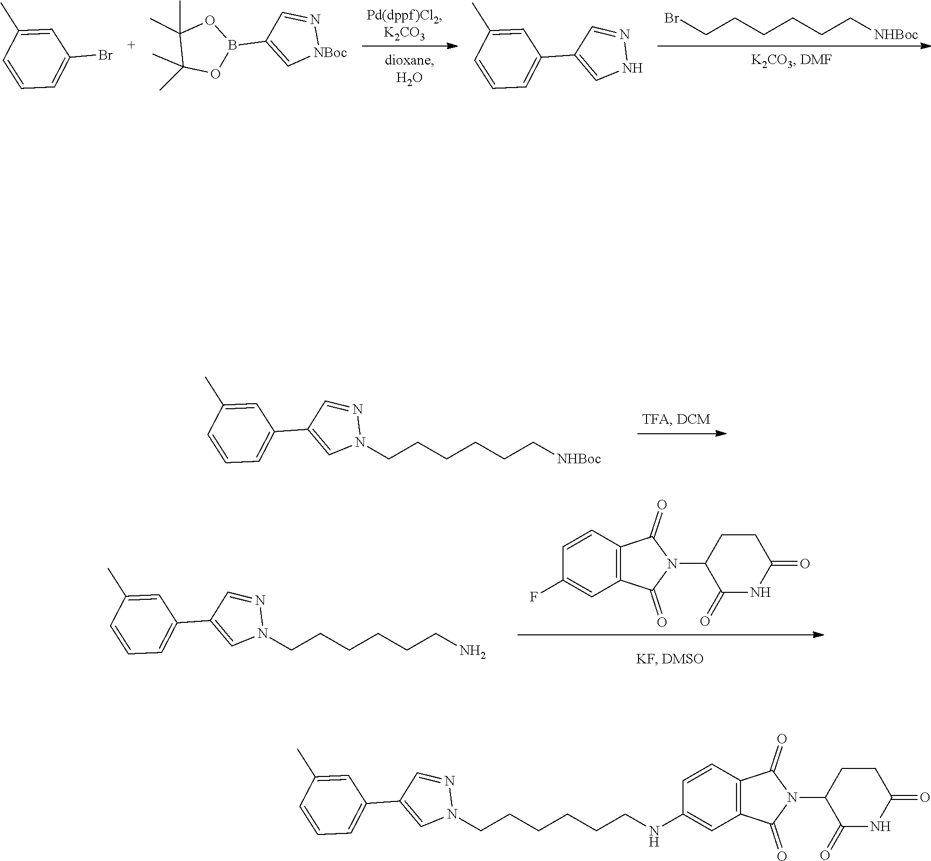

GS-497 was synthesized following the similar procedures as described for GS-498 (2.3 mg). MS (ESI) m/z=514.4 [M+H]⁺.

### Example 456. 2-(2,6-Dioxopiperidin-3-yl)-5-((6-(4-(6-methylpyridin-2-yl)-1H-pyrazol-1-yl)hexyl)amino)isoindoline-1,3-dione (GS-498)

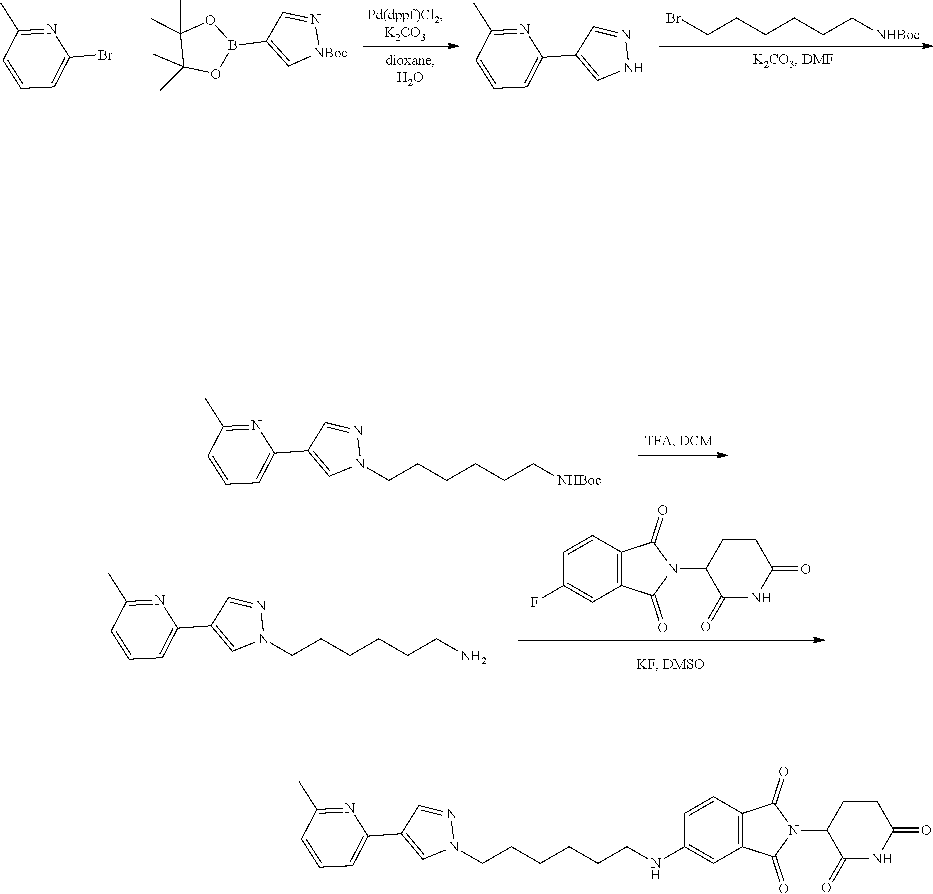

Step 1. Synthesis of 2-methyl-6-(1H-pyrazol-4-yl)pyridine

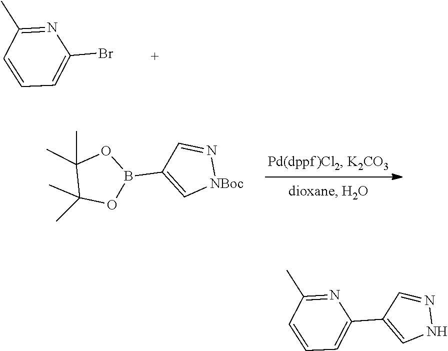

A solution of 2-bromo-6-methyl-pyridine (150 mg, 871.98 μmol), tert-butyl 4-(4,4,5,5-tetramethyl-1,3,2-dioxaborolan-2-yl)pyrazole-1-carboxylate (38.74 mg, 1.31 mmol), Pd(dppf)Cl₂ (63.80 mg, 87.20 μmol) and K₂CO₃ (241.03 mg, 1.74 mmol) in water (1 mL) and dioxane (4 mL) was stirred at 80° C. under N₂ for 2 h. Then, the mixture was concentrated, and the resulting residue was purified by silica gel chromatography to provide the title compound (50 mg, yield: 36%). MS (ESI) m/z=159.9 [M+H]⁺.

Step 2. Synthesis of tert-butyl (6-(4-(6-methylpyridin-2-yl)-1H-pyrazol-1-yl)hexyl)carbamate

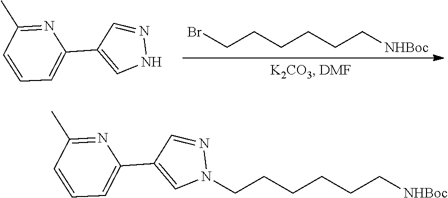

A solution of 2-methyl-6-(1H-pyrazol-4-yl)pyridine (30 mg, 188.46 mol), tert-butyl N-(6-bromohexyl)carbamate (58.09 mg, 207.30 μmol) and K₂CO₃ (52.09 mg, 376.91 mol) in DMF (1 mL) was stirred at 70° C. under N₂ for 2 h. The mixture was concentrated, and the resulting residue was purified by reverse phase chromatography to provide the title compound (50 mg, yield: 74%). MS (ESI) mz=359.5 [M+H]⁺.

Step 3. Synthesis of 6-(4-(6-methylpyridin-2-yl)-1H-pyrazol-1-yl)hexan-1-amine

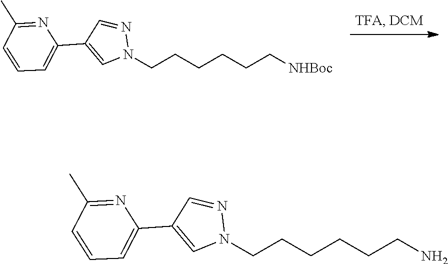 [SMILES: Cc1cccc(-c2cnn(CCCCCCNC(=O)OC(C)(C)C)c2)n1 ; Cc1cccc(-c2cnn(CCCCCCN)c2)n1]

A solution of tert-butyl(6-(4-(6-methylpyridin-2-yl)-1H-pyrazol-1-yl)hexyl)carbamate (50 mg, 139.48 μmol) in DCM (3 mL) and TFA (1 mL) was stirred at rt for 2 h. Then, the mixture was concentrated, and the resulting residue was purified by reverse phase chromatography to provide the title compound (35 mg, yield: 97%). MS (ESI) m/z=259.5 [M+H]⁺.

Step 4. Synthesis of 2-(2,6-dioxopiperidin-3-yl)-5-((6-(4-(6-methylpyridin-2-yl)-1H-pyrazol-1-yl)hexyl)amino)isoindoline-1,3-dione

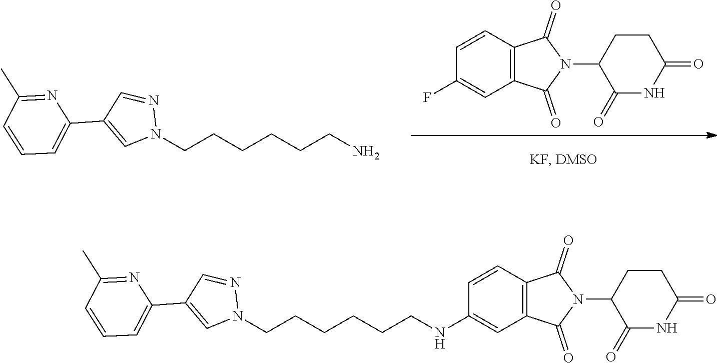 [SMILES: Cc1cccc(-c2cnn(CCCCCCN)c2)n1 ; O=C1CCC(N2C(=O)c3ccc(F)cc3C2=O)C(=O)N1 ; Cc1cccc(-c2cnn(CCCCCCNc3ccc4c(c3)C(=O)N(C3CCC(=O)NC3=O)C4=O)c2)n1]

A solution of 6-(4-(6-methylpyridin-2-yl)-1H-pyrazol-1-yl)hexan-1-amine (10 mg, 38.71 mol), 2-(2,6-dioxo-3-piperidyl)-5-fluoro-isoindoline-1,3-dione (16.04 mg, 58.06 μmol) and KF (2.25 mg, 38.71 μmol) in DMSO (1 mL) was stirred at 130° C. under tmicrowave irradiation for 15 min. Then, the mixture was purified by reverse phase chromatography to provide the title compound (1.0 mg, yield: 5%). MS (ESI) m/z=515.4 [M+H]9.

### Example 457. 2-(2,6-Dioxopiperidin-3-yl)-5-((6-(4-(5-methylpyridin-3-yl)-1H-pyrazol-1-yl)hexyl)amino)isoindoline-1,3-dione (GS-499)

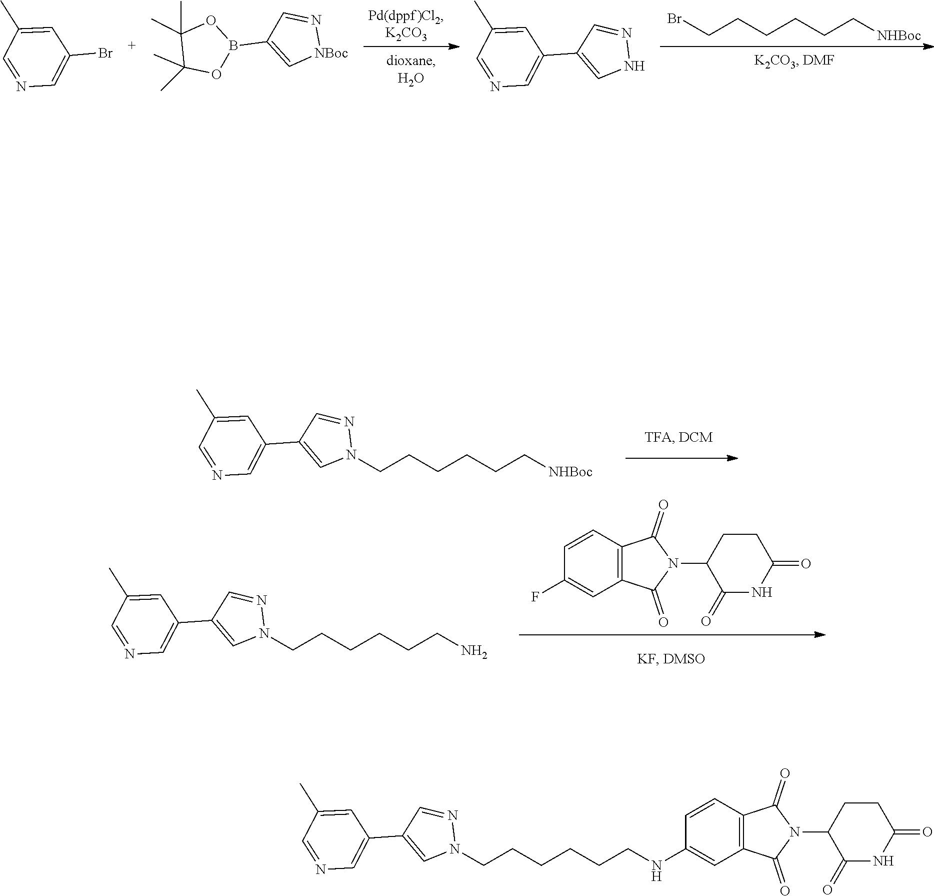

GS-499 was synthesized following the similar procedures as described for GS-498 (1.2 mg). MS (ESI) m/z=515.4 [M+H]⁺.

### Example 458. N-(2-(2,6-Dioxopiperidin-3-yl)-1-oxoisoindolin-5-yl)-2,2-difluoro-2-(3-(4-(4-(quinoxalin-2-yl)-1H-pyrazol-1-yl)piperidin-1-yl)phenyl)acetamide (GS-500)

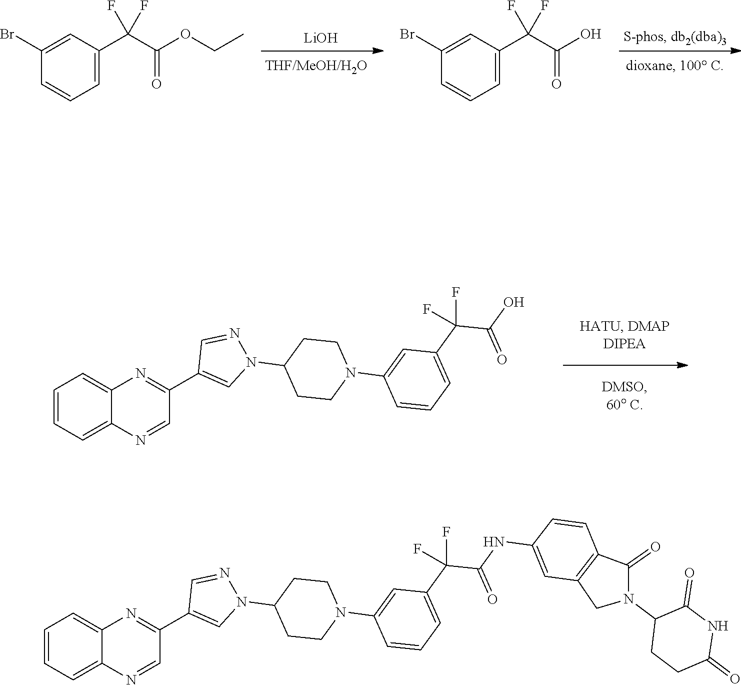

Step 1. Synthesis of 2-(3-bromophenyl)-2,2-difluoroacetic acid

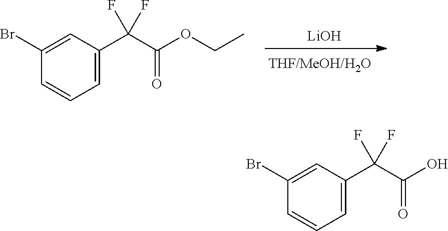

To a solution of ethyl 2-(3-bromophenyl)-2,2-difluoro-acetate (200 mg, 716 μmol) in THF (0.5 mL), water (0.2 mL) and methanol (0.5 mL) was added LiOH (85 mg, 3.5 mmol). The reaction mixture was stirred at rt for 1 h. The mixture was purified by reverse phase chromatography (0-70% MeCN in H₂O) to provide the title compound (100 mg, yield: 55%) as bright oil. MS (ESI) m/z=250.8 [M−H]⁻.

Step 2. Synthesis of 2,2-difluoro-2-(3-(4-(4-(quinoxalin-2-yl)-1H-pyrazol-1-yl)piperidin-1-yl)phenyl)acetic acid

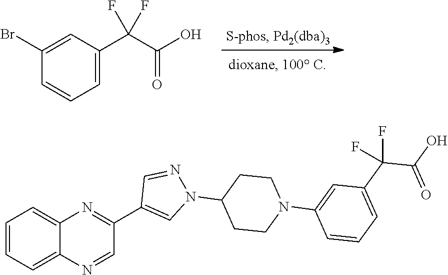 [SMILES: O=C(O)C(F)(F)c1cccc(Br)c1 ; "S-phos, Pd2(dba)3" ; O=C(O)C(F)(F)c1cccc(N2CCC(n3cc(-c4cnc5ccccc5n4)cn3)CC2)c1]

To a mixture of 2-(3-bromophenyl)-2,2-difluoroacetic acid (100 mg, 398 μmol) and 2-(1-(piperidin-4-yl)-1H-pyrazol-4-yl)quinoxaline (133 mg, 478 μmol) in dioxane (5 mL) were added s-phos (32 mg, 79 μmol), Pd₂(dba)₃ (22 mg, 39 μmol) and t-BuOK (89 mg, 796 μmol) under Ar. The reaction was stirred at 100° C. overnight. The mixture was quenched with water, and extracted with EtOAc. The organic phase was concentrated and purified by reverse phase chromatography (0-70% MeCN in H₂O) to provide the title compound (90 mg, yield: 50%) as yellow oil. MS (ESI) m/z=450.3 [M+H]⁺.

Step 3. Synthesis of N-(2-(2,6-dioxopiperidin-3-yl)-1-oxoisoindolin-5-yl)-2,2-difluoro-2-(3-(4-(4-(quinoxalin-2-yl)-1H-pyrazol-1-yl)piperidin-1-yl)phenyl)acetamide

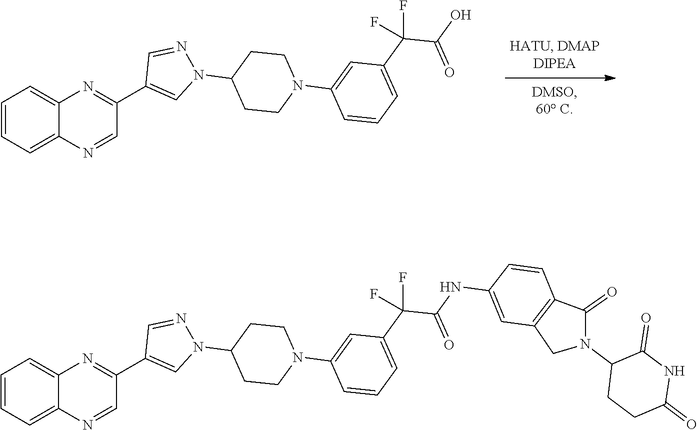 [SMILES: O=C(O)C(F)(F)c1cccc(N2CCC(n3cc(-c4cnc5ccccc5n4)cn3)CC2)c1 ; O=C1CCC(N2Cc3cc(NC(=O)C(F)(F)c4cccc(N5CCC(n6cc(-c7cnc8ccccc8n7)cn6)CC5)c4)ccc3C2=O)C(=O)N1]

To a mixture of 2,2-difluoro-2-(3-(4-(4-(quinoxalin-2-yl)-1H-pyrazol-1-yl)piperidin-1-yl)phenyl)acetic acid (60 mg, 133 μmol) and 3-(5-amino-1-oxo-isoindolin-2-yl)piperidine-2,6-dione (41 mg, 160 μmol) in DMSO (0.5 mL) were added HATU (101 mg, 266 μmol) and DMAP (2 mg, 13 μmol). The reaction mixture was stirred at 60° C. overnight. The reaction was purified by reverse phase chromatography (0-70% MeCN in H₂O) to provide the title compound (4.5 mg, yield: 5%) as gray solid. MS (ESI) m/z=691.5 [M+H]⁺.

### Example 459. N-(2-(2,6-Dioxopiperidin-3-yl)-1-oxoisoindolin-5-yl)-2-(3-(4-phenylpiperidin-1-yl)phenyl)acetamide (GS-501)

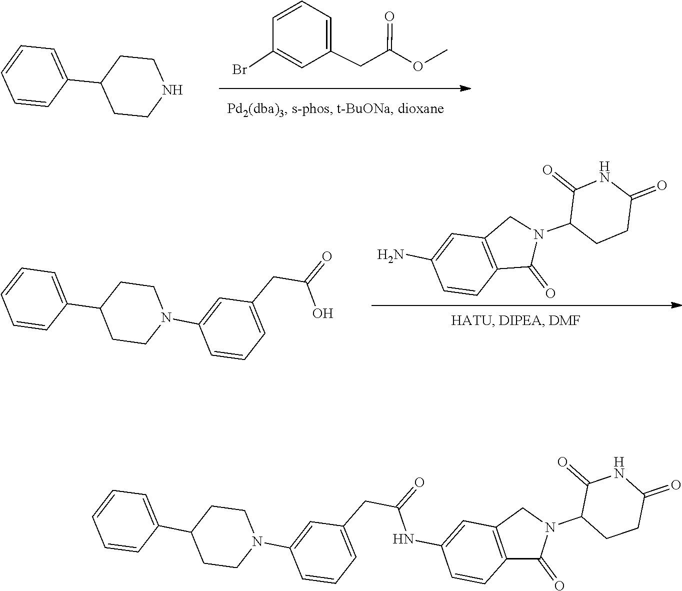 [SMILES: c1ccc(C2CCNCC2)cc1 ; COC(=O)Cc1cccc(Br)c1 ; O=C(O)Cc1cccc(N2CCC(c3ccccc3)CC2)c1 ; Nc1ccc2c(c1)CN(C1CCC(=O)NC1=O)C2=O ; O=C1CCC(N2Cc3cc(NC(=O)Cc4cccc(N5CCC(c6ccccc6)CC5)c4)ccc3C2=O)C(=O)N1]

Step 1. Synthesis of 2-(3-(4-phenylpiperidin-1-yl)phenyl)acetic acid

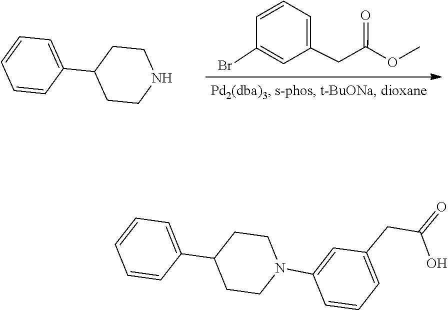 [SMILES: c1ccc(C2CCNCC2)cc1 ; COC(=O)Cc1cccc(Br)c1 ; O=C(O)Cc1cccc(N2CCC(c3ccccc3)CC2)c1]

To a solution of 4-phenylpiperidine (100 mg, 0.62 mmol) in dioxane (2 mL) were added methyl 2-(3-bromophenyl)acetate (141 mg, 0.62 mmol), Pd₂(dba)₃ (56 mg, 0.06 mmol), L-proline (50 mg, 0.12 mmol), t-BuONa (119 mg, 1.24 mmol). The mixture was stirred at 90° C. for 1 h under N₂. The mixture was purified by reverse phase chromatography (MeOH:H₂O=1:1) to provide the title compound (14 mg, yield: 8%) as yellow oil. MS (ESI) m/z=296.4 [M+H]⁺.

Step 2. Synthesis of N-(2-(2,6-dioxopiperidin-3-yl)-1-oxoisoindolin-5-yl)-2-(3-(4-phenylpiperidin-1-yl)phenyl)acetamide

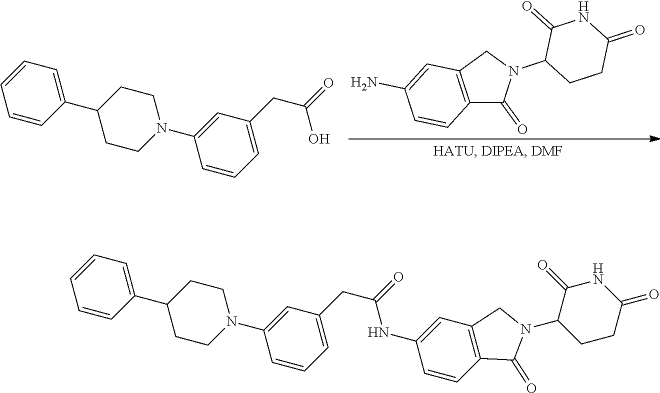

To a solution of 2-(3-(4-phenylpiperidin-1-yl)phenyl)acetic acid (14 mg, 0.05 mmol) in DMF (1 mL) were added 3-(5-amino-1-oxoisoindolin-2-yl)piperidine-2,6-dione (13 mg, 0.05 mg), HATU (28 mg, 0.075 mmol) and DIPEA (19 mg, 0.15 mmol). The mixture was stirred at rt for 2 h, and then purified by reverse phase chromatography (MeOH:H₂O=1:1) to provide the title compound (3 mg, yield: 12%) as yellow solid. MS (ESI) m/z=537.4 [M+H]⁺.

### Example 460. 2-(2,6-Dioxopiperidin-3-yl)-5-((6-(4-(3-methylquinoxalin-2-yl)-1H-pyrazol-1-yl)hexyl)amino)isoindoline-1,3-dionee (GS-502)

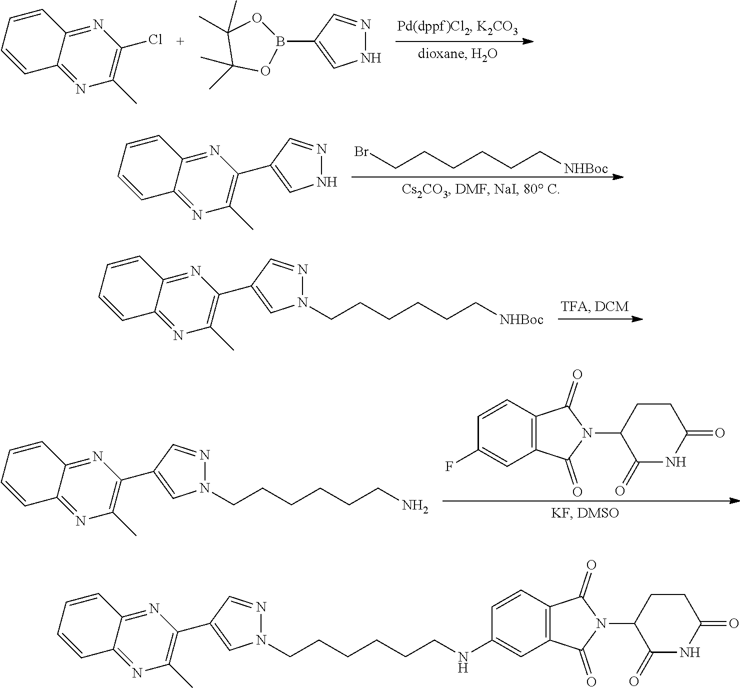

GS-502 was synthesized following the similar procedures as described for GS-498 (80 mg). MS (ESI) m/z=566.4 [M+H]⁺.

### Example 461. 5-((6-(4-(7-Bromoquinoxalin-2-yl)-1H-pyrazol-1-yl)hexyl)amino)-2-(2,6-dioxopiperidin-3-yl)isoindoline-1,3-dione (GS-503)

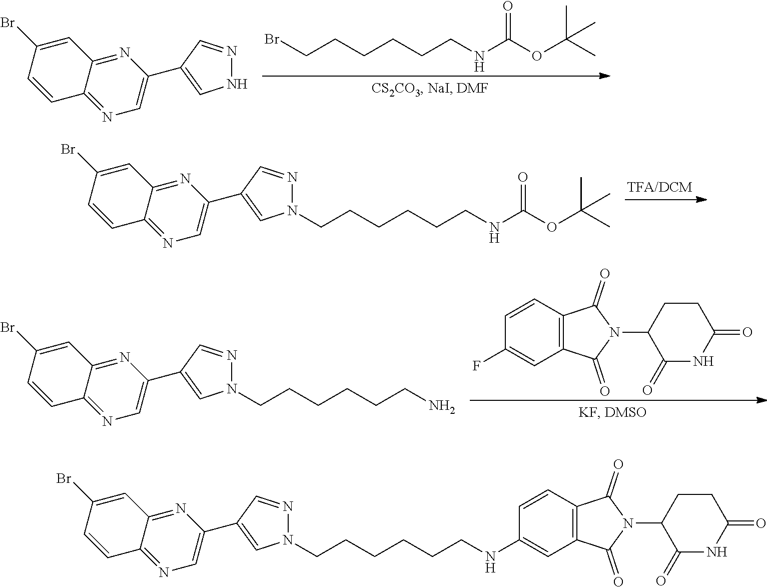

GS-503 was synthesized following the similar procedures as described for GS-498 (38 mg) as white solid. MS (ESI) m/z=632.2 [M+H]⁺.

### Example 462. 2-(2,6-Dioxopiperidin-3-yl)-5-((3-(3-(4-phenylpiperidin-1-yl)phenyl)propyl)amino)isoindoline-1,3-dione (GS-504)

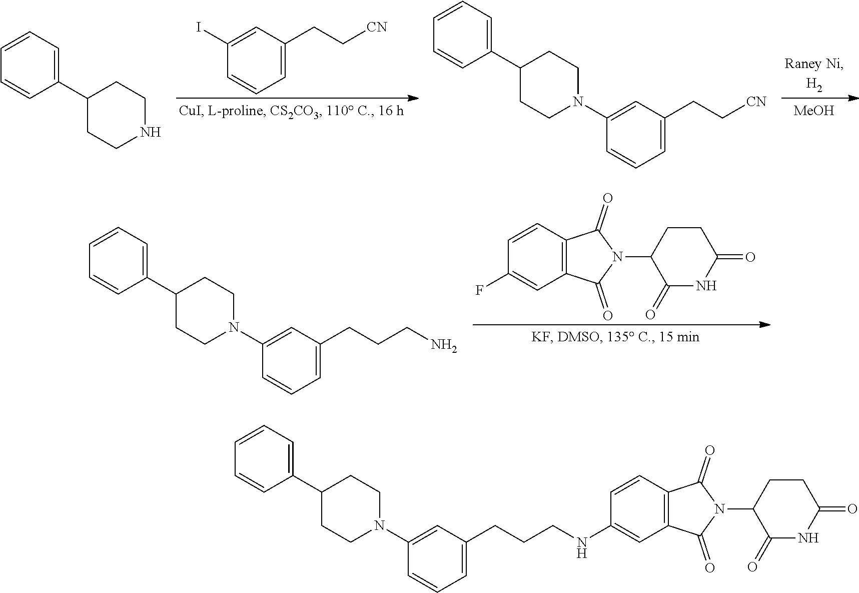

Step 1. Synthesis of 3-(3-(4-phenylpiperidin-1-yl)phenyl)propanenitrile

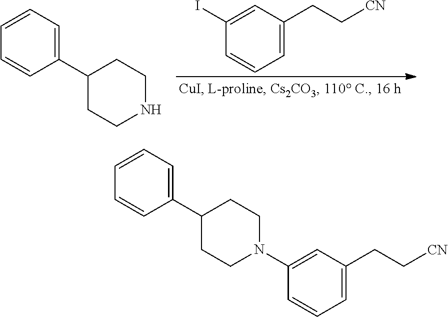

To a mixture of 4-phenylpiperidine (320 mg, 2 mmol) in DMF (5 mL) were added 3-(3-iodophenyl)propanenitrile (514 mg, 2 mmol), CuI (76 mg, 0.4 mmol), L-proline (68 mg, 0.6 mmol) and Cs₂CO₃ (1.3 g, 4 mmol). The mixture was stirred at 110° C. for 16 h under N₂ atmosphere. The mixture was purified by reverse phase chromatography (MeOH:H₂O=2:1) to provide the title compound (29 mg, yield: 5%) as yellow oil. MS (ESI) m/z=291.4 [M+H]⁺.

Step 2. Synthesis of 3-(3-(4-phenylpiperidin-1-yl)phenyl)propan-1-amine

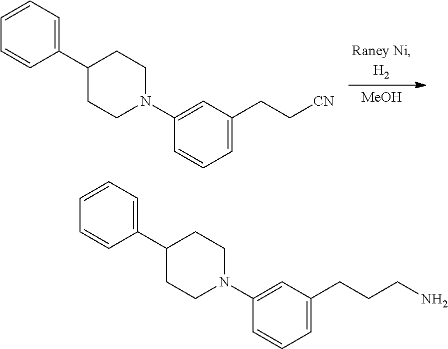 [SMILES: "MeOH" ; N#CCCc1cccc(N2CCC(c3ccccc3)CC2)c1 ; NCCCc1cccc(N2CCC(c3ccccc3)CC2)c1]

To a solution of 3-(3-(4-phenylpiperidin-1-yl)phenyl)propanenitrile (29 mg, 0.1 mmol) in methanol (5 mL) was added Raney-Ni (100 mg). The mixture was stirred at rt for 2 h under H₂ atomosphere. The mixture was filtered, and the filtrate was concentrated to provide the title compound (18 mg, yield: 61%) as colorless oil. MS (ESI) m/z=295.4 [M+H]⁺.

Step 3. Synthesis of 2-(2,6-dioxopiperidin-3-yl)-5-((3-(3-(4-phenylpiperidin-1-yl)phenyl)propyl)amino)isoindoline-1,3-dione

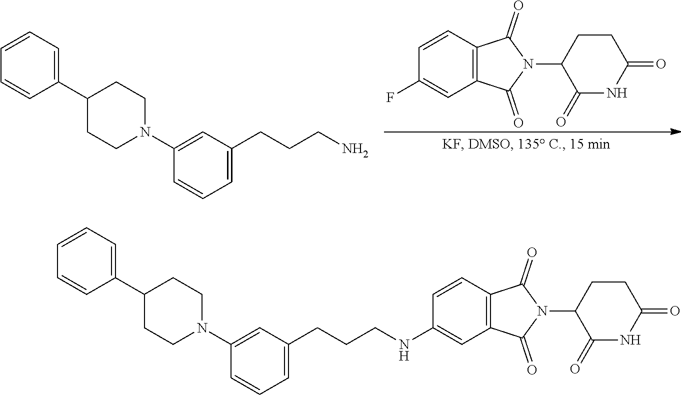 [SMILES: NCCCc1cccc(N2CCC(c3ccccc3)CC2)c1 ; O=C1CCC(N2C(=O)c3ccc(F)cc3C2=O)C(=O)N1 ; O=C1CCC(N2C(=O)c3ccc(NCCCc4cccc(N5CCC(c6ccccc6)CC5)c4)cc3C2=O)C(=O)N1]

To a solution of 3-(3-(4-phenylpiperidin-1-yl)phenyl)propan-1-amine (18 mg, 0.06 mmol) in DMSO (1 mL) were added 2-(2,6-dioxopiperidin-3-yl)-5-fluoroisoindoline-1,3-dione (20 mg, 0.07 mmol) and KF (7 mg, 0.12 mmol). The mixture was stirred at 135° C. for 15 min. The mixture was purified by reverse phase chromatography (MeOH:H₂O=1:1) to provide the title compound (3 mg, yield: 9%) as yellow solid. MS (ESI) m/z=551.5 [M+H]⁺.

### Example 463. 2-(2,6-Dioxopiperidin-3-yl)-5-((3-(3-fluoro-5-(4-phenylpiperidin-1-yl)phenyl)propyl)amino)isoindoline-1,3-dione (GS-505)

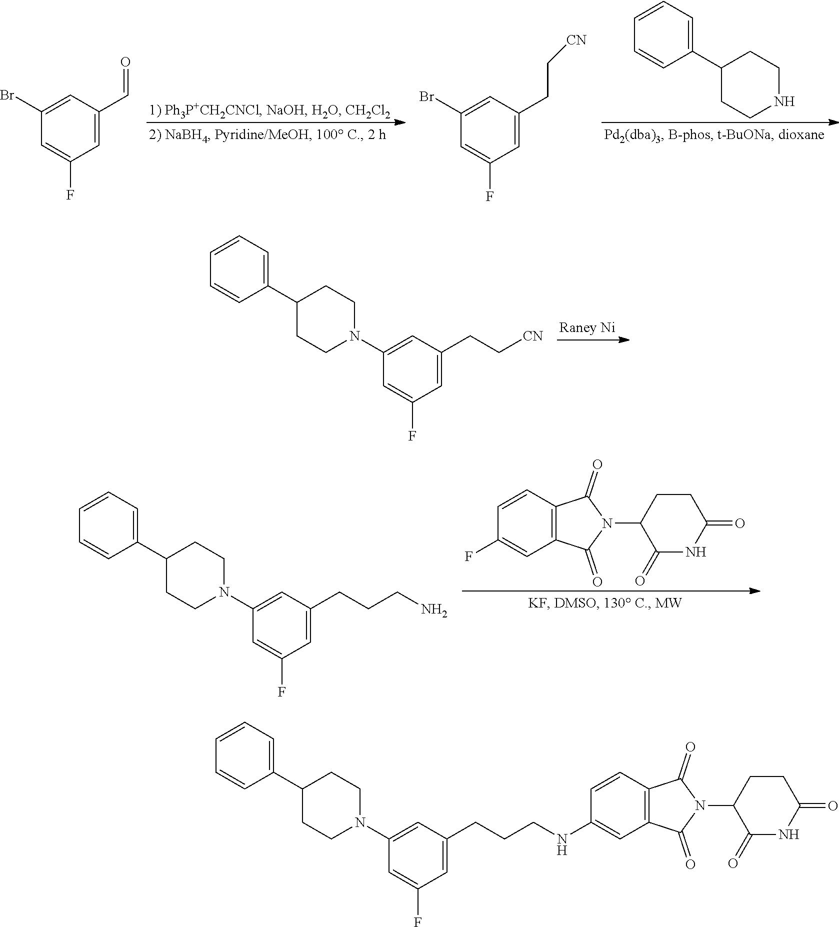

Step 1. Synthesis of 3-(3-bromo-5-fluorophenyl)propanenitrile

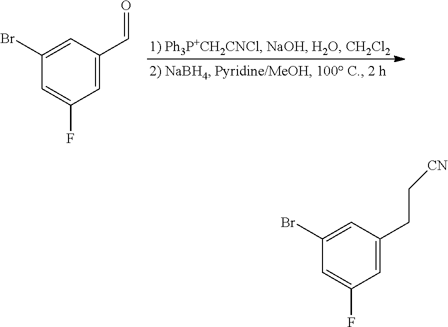 [SMILES: O=Cc1cc(F)cc(Br)c1 ; N#CCCc1cc(F)cc(Br)c1]

To a solution of 3-bromo-5-fluorobenzaldehyde (1 g, 4.93 mmol) in DCM (50 mL) was added aq. NaOH solution (30%, 50 mL), followed by (cyanomethyl)triphenylphosphonium chloride (2.66 g, 7.88 mmol). After the reaction was stirred at rt for 30 min, it was quenched with water (100 mL). The mixture was extracted with DCM (50 mL). The organic phase was concentrated, and the residue was dissolved in CH₃OH (30 mL) and pyridine (30 mL) under N₂. To this solution was added NaBH₄ (364.52 mg, 9.85 mmol). After the mixture was stirred at 100° C. for 2 h, the mixture was poured into ice/water, acidified with aq. 1 M HCl solution to pH=1. The mixture was extracted with EtOAc (50 mL). The organic phase was concentrated, and the resulting residue was purified by prep-TLC (petroleum ether:EtOAc=5:1) to provide the title compound (310 mg, yield: 28%) as yellow oil.

Step 2. Synthesis of 3-(3-fluoro-5-(4-phenylpiperidin-1-yl)phenyl)propanenitrile

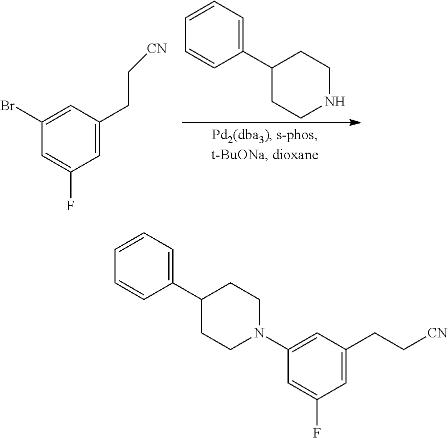

To a solution of 3-(3-bromo-5-fluorophenyl)propanenitrile (50 mg, 219.24 μmol) in dioxane (2 mL) were added 4-phenylpiperidine (35.35 mg, 219.24 μmol), tris(dibenzylideneacetone)dipalladium (20.06 mg, 21.92 μmol), 2-dicyclohexylphosphino-2′,6′-dimethoxy-1,1′-biphenyl (18.00 mg, 43.85 μmol) and sodium tert-butoxide (42.14 mg, 438.48 μmol). The mixture was stirred at 120° C. for 30 min, and then purified by reverse phase chromatography (MeOH:H₂O=2:1) to provide the title compound (40 mg, yield: 59%) as yellow solid. MS (ESI) m/z=309.4 [M+H]⁺.

Step 3. Synthesis of 3-(3-fluoro-5-(4-phenylpiperidin-1-yl)phenyl)propan-1-amine

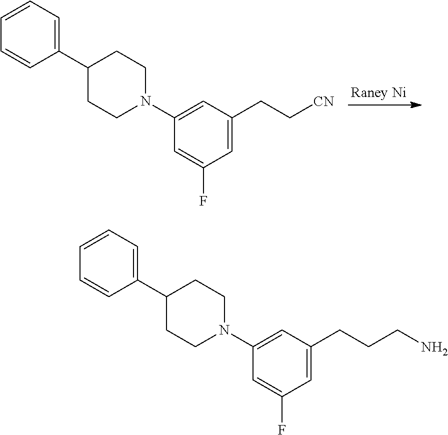 [SMILES: N#CCCc1cc(F)cc(N2CCC(c3ccccc3)CC2)c1 ; NCCCc1cc(F)cc(N2CCC(c3ccccc3)CC2)c1]

To a solution of 3-(3-fluoro-5-(4-phenylpiperidin-1-yl)phenyl)propanenitrile (36 mg, 116.73 μmol) in CH₃OH (5 mL) was added Raney Ni (100 mg). The mixture was stirred at it for 30 min under H₂ atomosphere. The mixture was filtered, and concentrated to provide the title compound (21 mg, yield: 58%) as yellow oil. MS (ESI) m/z=313.4 [M+H]⁺.

Step 4. Synthesis of 2-(2,6-dioxopiperidin-3-yl)-5-((3-(3-(4-phenylpiperidin-1-yl)phenyl)propyl)amino)isoindoline-1,3-dione

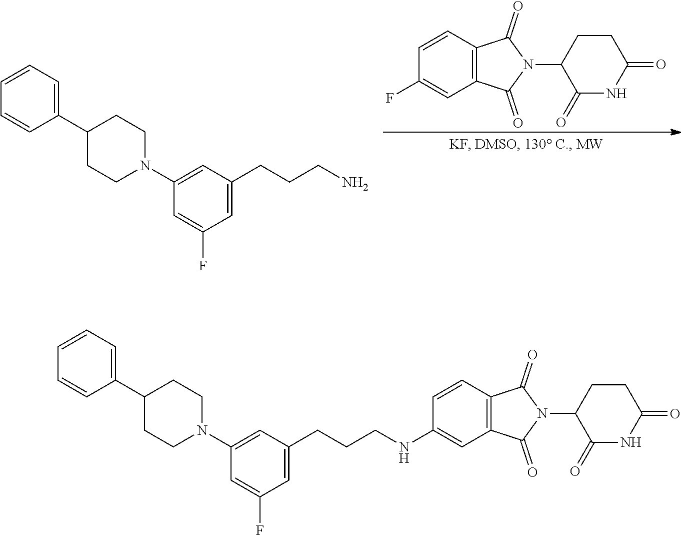 [SMILES: NCCCc1cc(F)cc(N2CCC(c3ccccc3)CC2)c1 ; O=C1CCC(N2C(=O)c3ccc(F)cc3C2=O)C(=O)N1 ; O=C1CCC(N2C(=O)c3ccc(NCCCc4cc(F)cc(N5CCC(c6ccccc6)CC5)c4)cc3C2=O)C(=O)N1]

To a solution of 3-(3-fluoro-5-(4-phenylpiperidin-1-yl)phenyl)propan-1-amine (21 mg, 67.22 μmol) in DMSO (1 mL) were added 2-(2,6-dioxopiperidin-3-yl)-5-fluoroisoindoline-1,3-dione (18.57 mg, 67.22 μmol) and KF (7.81 mg, 134.43 μmol). The mixture was stirred at 135° C. for 15 min, and then purified by reverse phase chromatography to provide the title compound (10 mg, yield: 26%) as yellow solid. MS (ESI) m/z=569.5 [M+H]⁺.

### Example 464. 2-(2,6-Dioxopiperidin-3-yl)-5-((3-(4-fluoro-3-(4-phenylpiperidin-1-yl)phenyl)propyl)amino)isoindoline-1,3-dione (GS-506)

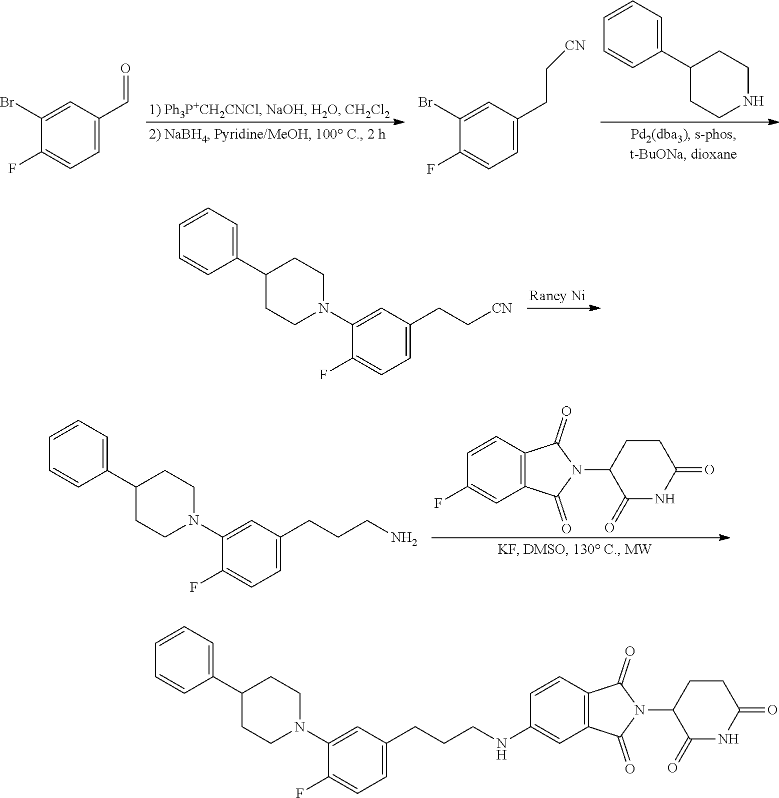

GS-506 was synthesized following the similar procedures as described for GS-505 (10 mg) as yellow solid. MS (ESI) m/z=569.5 [M+H]⁺.

### Example 465. 2-(2,6-Dioxopiperidin-3-yl)-5-((3-(2-fluoro-3-(4-phenylpiperidin-1-yl)phenyl)propyl)amino)isoindoline-1,3-dione (GS-507)

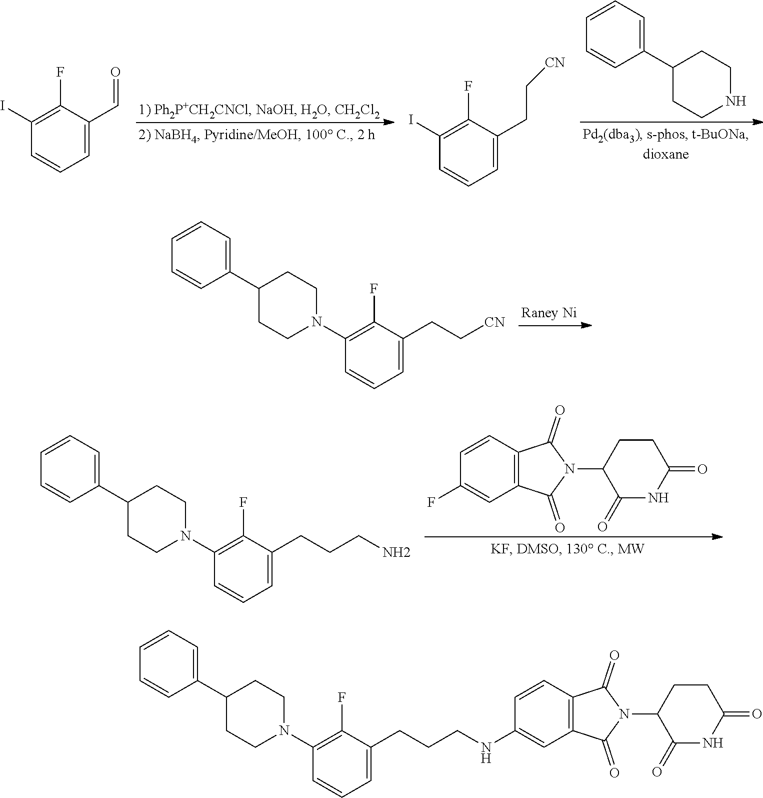

GS-507 was synthesized following the similar procedures as described for GS-505 (3 mg) as yellow solid. MS (ESI) m/z=569.5 [M+H]⁺.

### Example 466. 2-(2,6-Dioxopiperidin-3-yl)-5-((3-(4-fluoro-3-(4-phenylpiperidin-1-yl)phenyl)propyl)amino)isoindoline-1,3-dione (GS-508)

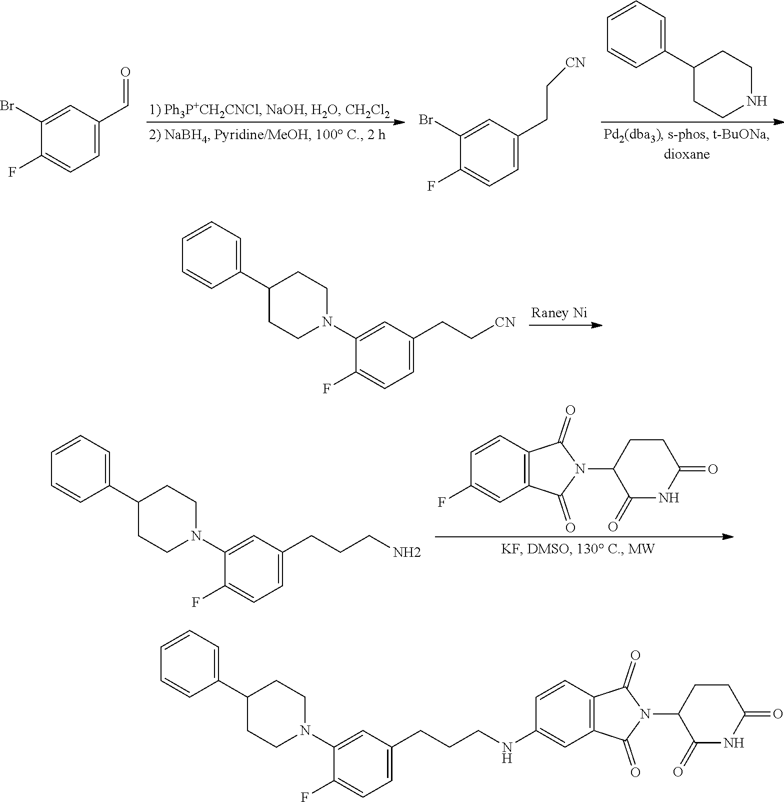

GS-508 was synthesized following the similar procedures as described for GS-505 (10 mg) as yellow solid. MS (ESI) m/z=569.5 [M+H]⁺.

### Example 467. N-((2-(2,6-Dioxopiperidin-3-yl)-1-oxoisoindolin-4-yl)methyl)-3-(4-(4-(quinoxalin-2-yl)-1H-pyrazol-1-yl)piperidin-1-yl)benzamide (GS-509)

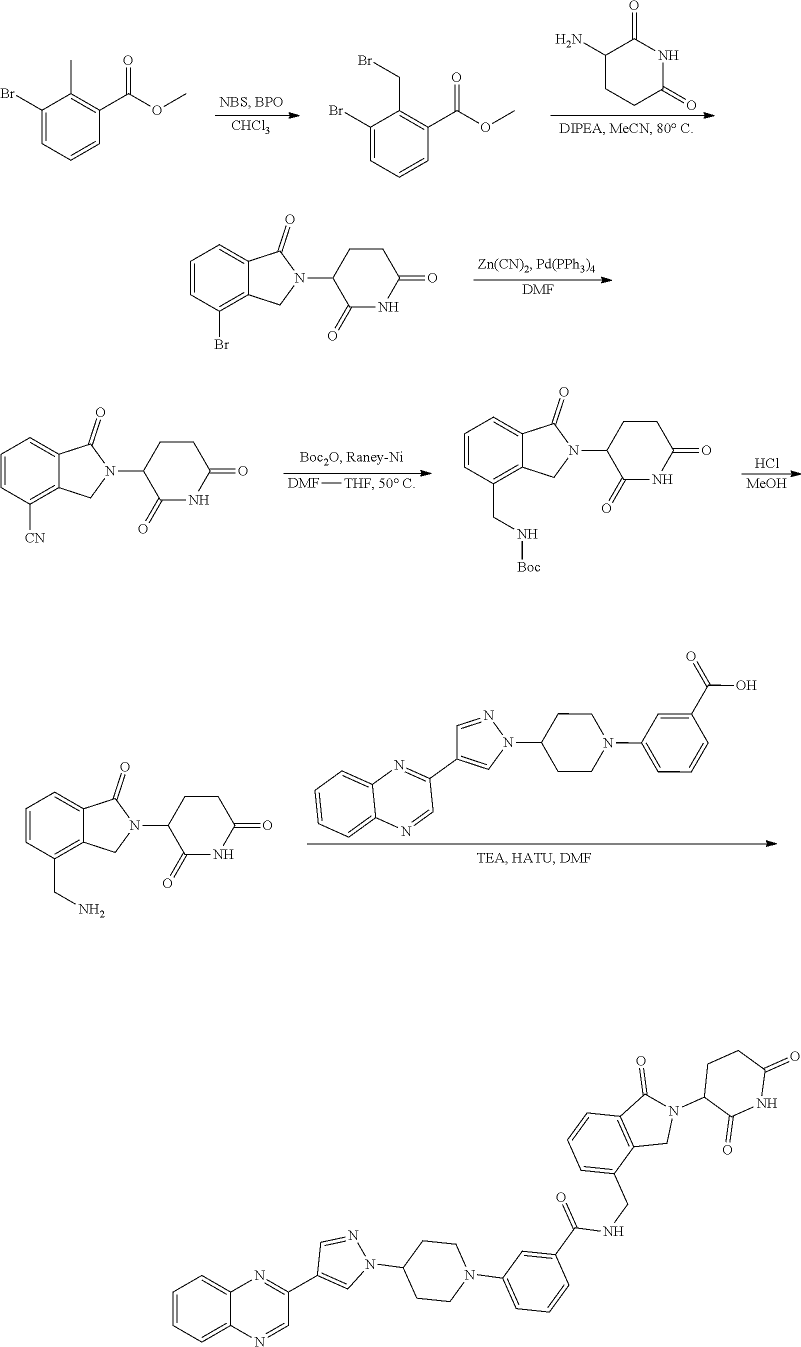

Step 1. Synthesis of methyl 3-bromo-2-(bromomethyl)benzoate

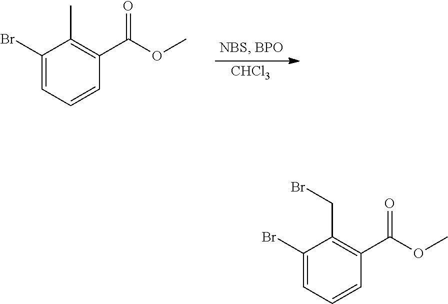

To a solution of methyl 3-bromo-2-methyl-benzoate (2.8 g, 12.22 mmol) in CHCl₃ (40 mL) was added 1-bromopyrrolidine-2,5-dione (2.61 g, 14.67 mmol) and BPO (296.09 mg, 1.22 mmol) at 25° C. The mixture was stirred at 75° C. for 18 h, and then cooled to 25° C. After concentration in vacuo, the mixture was purified by flash chromagraphy (petroleum ether:EtOAc=100:1-20:1) to provide the title compound (3.2 g, yield: 85%) as colorless oil.

Step 2. Synthesis of 3-(4-bromo-1-oxoisoindolin-2-yl)piperidine-2,6-dione

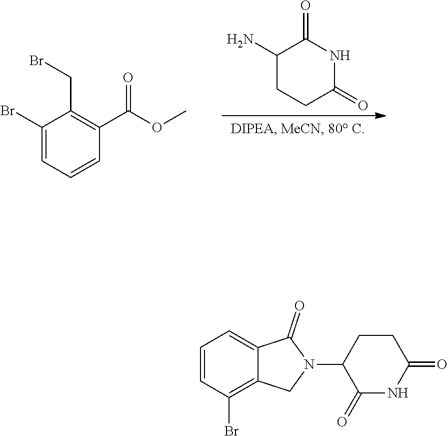 [SMILES: COC(=O)c1cccc(Br)c1CBr ; NC1CCC(=O)NC1=O ; O=C1CCC(N2Cc3c(Br)cccc3C2=O)C(=O)N1]

To a mixture of methyl 3-bromo-2-(bromomethyl)benzoate (3.2 g, 10.39 mmol) and 3-aminopiperidine-2,6-dione (HCl salt, 1.88 g, 11.43 mmol) in MeCN (100 mL) was added DIPEA (2.69 g, 20.78 mmol) at 25° C. After the mixture was stirred at 80° C. for 18 h, it was filtered. The filter cake was dried in vacuo to provide the title compound (2 g, yield: 60%) as white solid. MS (ESI) m/z=324.9 [M+H]⁺.

Step 3. Synthesis of 2-(2,6-dioxopiperidin-3-yl)-1-oxoisoindoline-4-carbonitrile

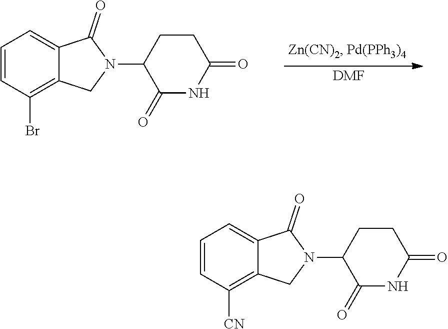

To a mixture of 3-(4-bromo-1-oxo-isoindolin-2-yl)piperidine-2,6-dione (1 g, 3.09 mmol) in DMF (20 mL) were added Zn(CN)₂ (3.63 g, 30.95 mmol) and Pd(PPh₃)₄ (1.07 g, 928.39 μmol) at 25° C. The mixture was stirred at 140° C. for 24 h under N₂. After the reaction was cooled to 25° C., it was concentrated and purified by silica gel chromatoprophy (petroleum ether:EtOAc=100:1˜0:1) to provide the title compound (0.4 g, yield: 48%) as white solid.

Step 4. Synthesis of tert-butyl ((2-(2,6-dioxopiperidin-3-yl)-1-oxoisoindolin-4-yl)methyl)carbamate

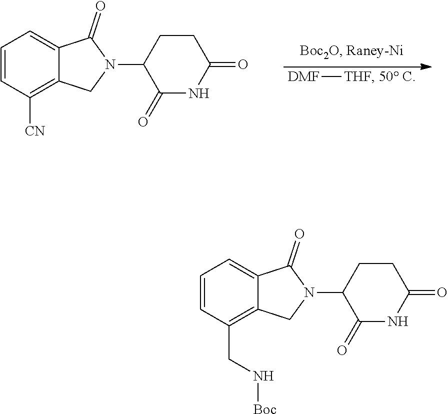

A mixture of 2-(2,6-dioxo-3-piperidyl)-1-oxo-isoindoline-4-carbonitrile (0.2 g, 742.79 μmol), Boc₂O (243.17 mg, 1.11 mmol), and Raney-Ni (200 mg, 742.79 μmol) in THF (2 mL) and DMF (2 mL) was stirred at 40° C. under H₂ for 18 h. The mixture was filtered. The filtrate was concentrated and purified by flash chromatography to provide the title compound (270 mg, yield: 97%) as white solid. MS (ESI) m/z=374.2 [M+H]⁺.

Step 5. Synthesis of 3-(4-(aminomethyl)-1-oxoisoindolin-2-yl)piperidine-2,6-dione

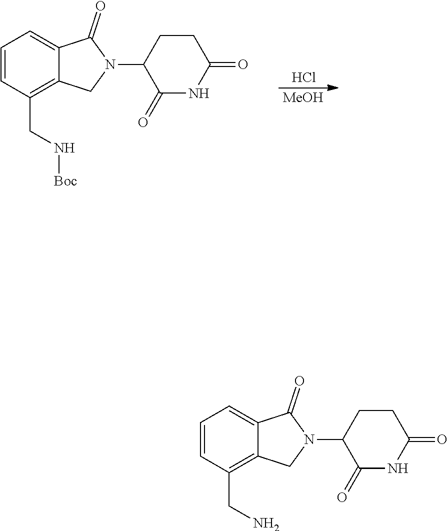 [SMILES: "MeOH" ; CC(C)(C)OC(=O)NCc1cccc2c1CN(C1CCC(=O)NC1=O)C2=O ; Cl ; NCc1cccc2c1CN(C1CCC(=O)NC1=O)C2=O]

To a solution of tert-butyl ((2-(2,6-dioxopiperidin-3-yl)-1-oxoisoindolin-4-yl)methyl)carbamate (100 mg, 267.81 μmol) in methanol (2 mL) was added aq. HCl solution (conc., 0.2 mL) at 25° C. The mixture was stirred for 2 h, and then concentrated in vacuo to provide the title compound (70 mg, yield: 96%) as white solid.

Step 6. Synthesis of N-((2-(2,6-dioxopiperidin-3-yl)-1-oxoisoindolin-4-yl)methyl)-3-(4-(4-(quinoxalin-2-yl)-1H-pyrazol-1-yl)piperidin-1-yl)benzamide

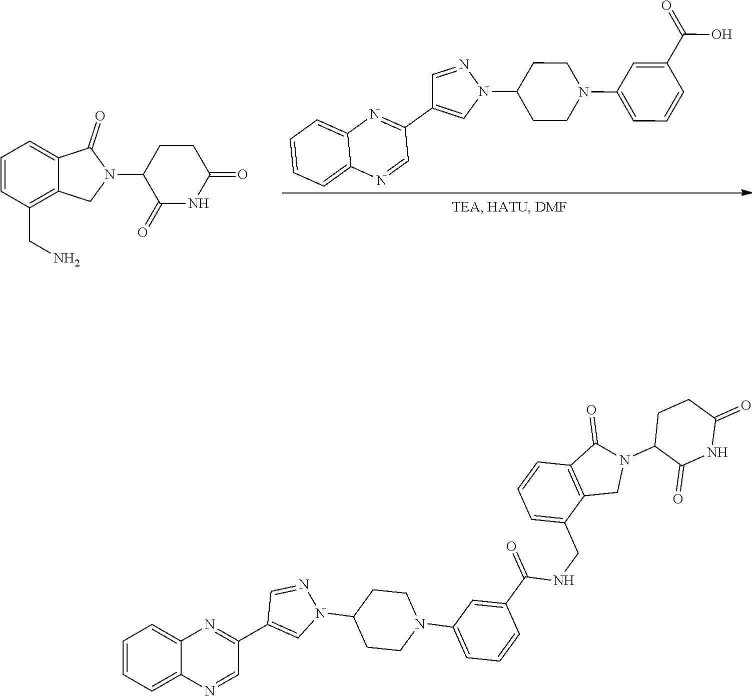 [SMILES: NCc1cccc2c1CN(C1CCC(=O)NC1=O)C2=O ; O=C(O)c1cccc(N2CCC(n3cc(-c4cnc5ccccc5n4)cn3)CC2)c1 ; O=C1CCC(N2Cc3c(CNC(=O)c4cccc(N5CCC(n6cc(-c7cnc8ccccc8n7)cn6)CC5)c4)cccc3C2=O)C(=O)N1]

To a mixture of 3-(4-(4-(quinoxalin-2-yl)-1H-pyrazol-1-yl)piperidin-1-yl)benzoic acid (see step 1 of GS-476 for synthesis, 10 mg, 25.03 μmol) and 3-(4-(aminomethyl)-1-oxoisoindolin-2-yl)piperidine-2,6-dione (6.82 mg, 25.03 μmol) in DMF (2 mL) were added TEA (7.60 mg, 75.10 μmol) and HATU (14.28 mg, 37.55 μmol) at 25° C. After the mixture was stirred for 2 h, it was concentrated and purified by flash chromatography to provide the title compound (2.2 mg, yield: 13%) as brown solid. MS (ESI) m/z=655.5 [M+H]⁺.

### Example 468. 5-((6-(3,5-Dimethyl-4-(quinoxalin-2-yl)-1H-pyrazol-1-yl)hexyl)amino)-2-(2,6-dioxopiperidin-3-yl)isoindoline-1,3-dione-(GS-510)

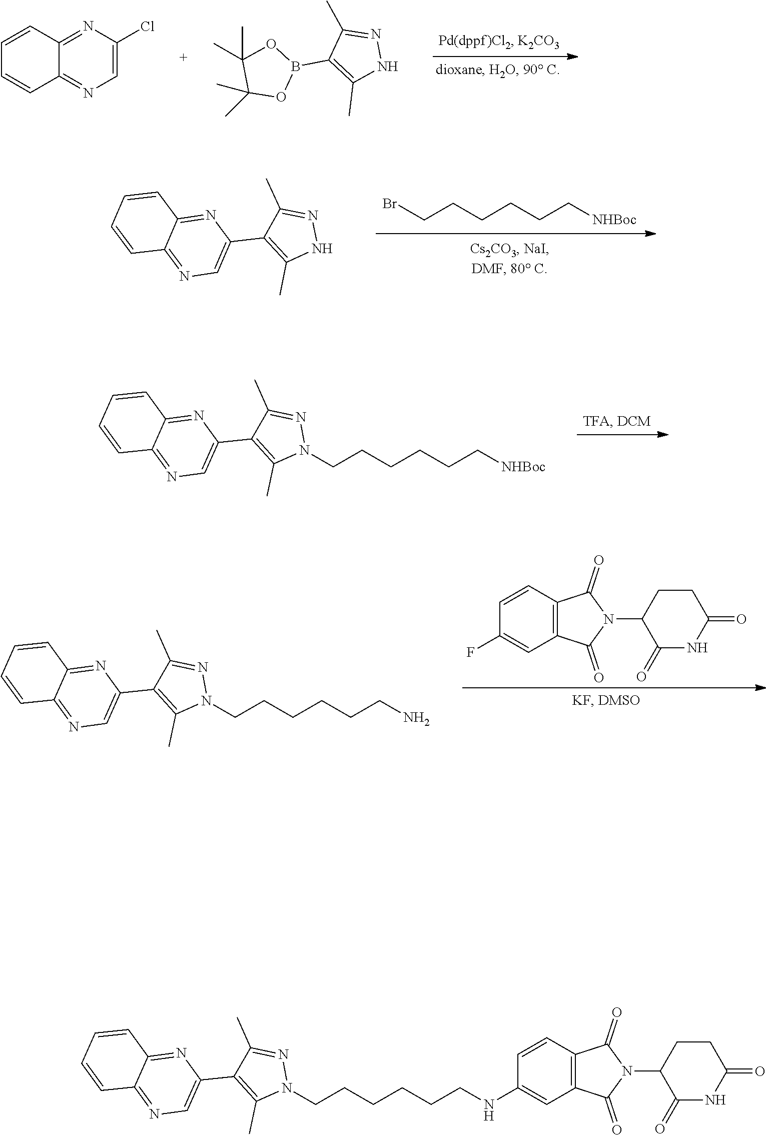

GS-510 was synthesized following the similar procedures as described for GS-498 (2.9 mg). MS (ESI) m/z=580.37 [M+H]⁺.

### Example 469 and 470. 2-(2,6-Dioxopiperidin-3-yl)-5-((6-(4-(8-methylquinoxalin-2-yl)-1H-pyrazol-1-yl)hexyl)amino)isoindoline-1,3-dione (GS-511) and 2-(2,6-dioxopiperidin-3-yl)-5-((6-(4-(5-methylquinoxalin-2-yl)-1H-pyrazol-1-yl)hexyl)amino)isoindoline-1,3-dione (GS-512)

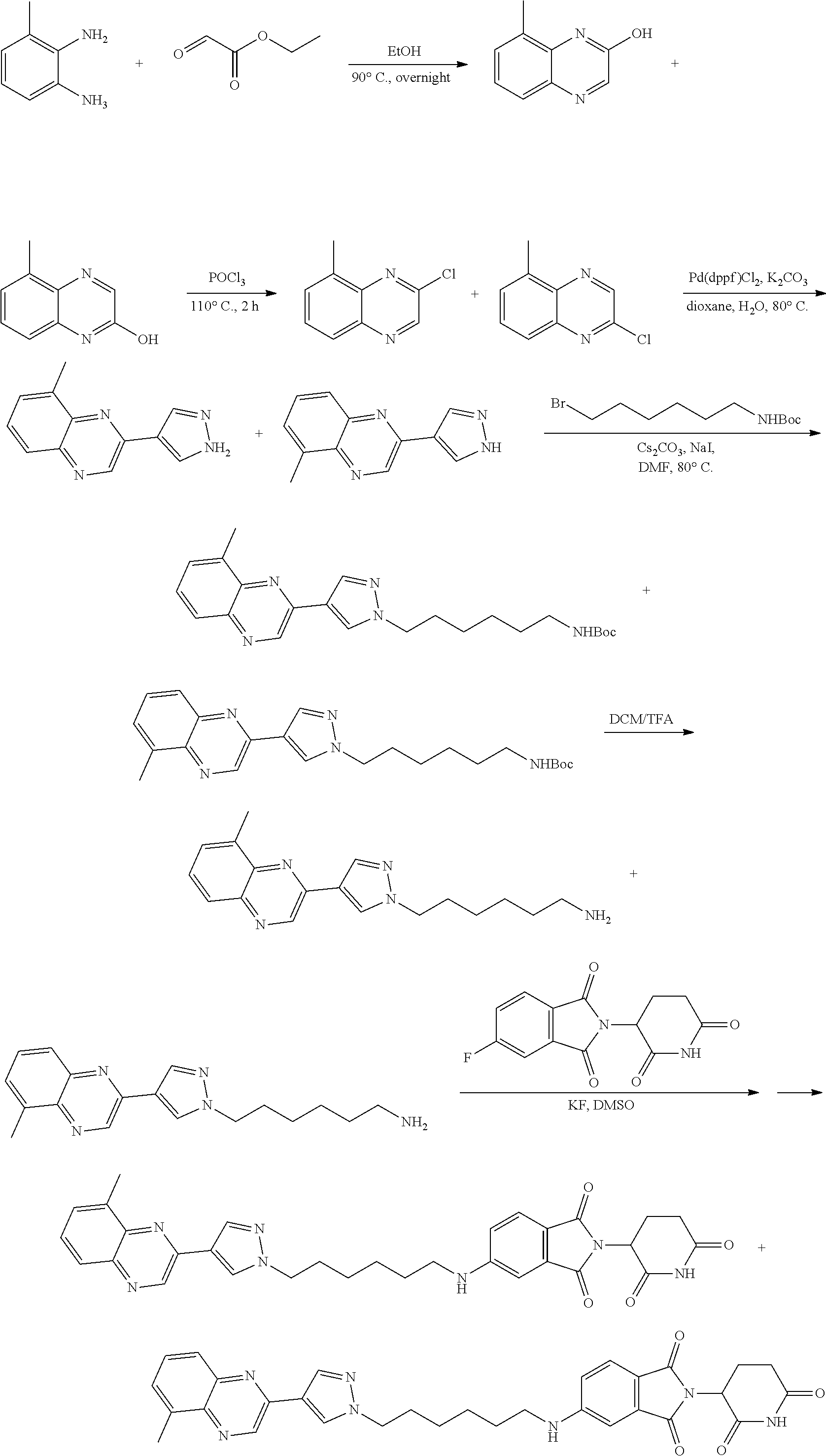

Step 1. Syntheses of 8-methylquinoxalin-2-ol and 5-methylquinoxalin-2-ol

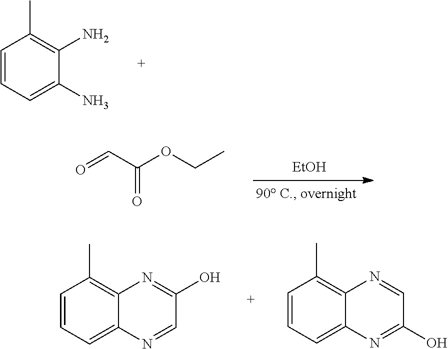

A solution of 3-methylbenzene-1,2-diamine (5 g, 40.93 mmol) and ethyl 2-oxoacetate (4.60 g, 45.02 mmol) in ethanol (50 mL) was stirred at 90° C. under N₂ overnight. The mixture was concentrated. The resulting residue was purified by silica gel chromatography to provide the title compounds as a mixture (4 g, yield: 61%). MS (ESI) m/z=160.8 [M+H]⁺.

Step 2. Syntheses of 2-chloro-8-methylquinoxaline and 2-chloro-5-methylquinoxaline

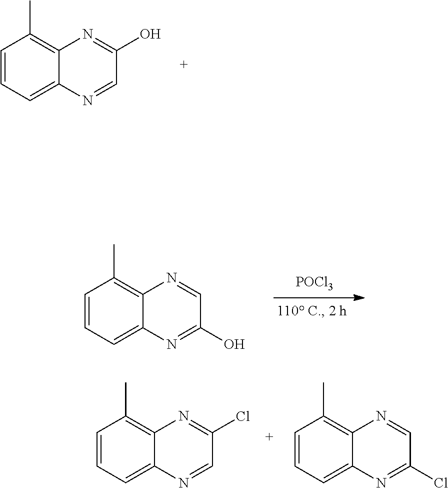

A solution of methylquinoxalin-2-ols (1 g, 6.24 mmol) in POCl₃ (5 mL) was stirred at 100° C. under N₂ for 2 h. After the mixture was cooled to rt, it was poured to ice/water (30 mL), and extracted with EtOAc. The organic phase was concentrated, and the resulting residue was purified by silica gel chromatography to provide the title compounds as a mixture (1 g, yield: 90%). MS (ESI) m/z=179.8 [M+H]⁺.

Step 3. Syntheses of 8-methyl-2-(1H-pyrazol-4-yl)quinoxaline and 5-methyl-2-(1H-pyrazol-4-yl)quinoxaline

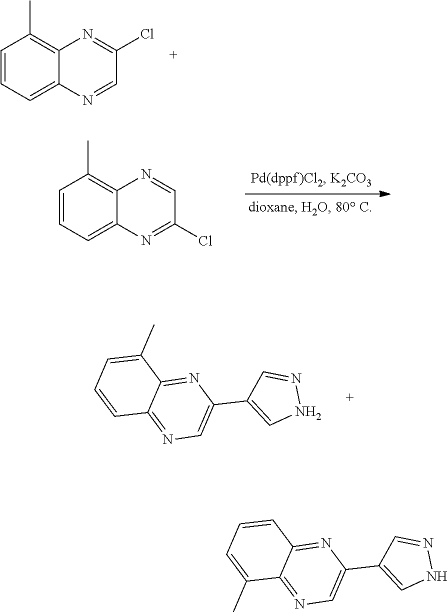

A solution of 2-chloro-methyl-quinoxalines (1 g, 5.60 mmol), Pd(dppf)Cl₂ (409.65 mg, 559.85 μmol), K₂CO₃ (1.55 g, 11.20 mmol) in dioxane (10 mL) and water (2.5 mL) was stirred at 80° C. under N₂ for 2 h. Then, the mixture was concentrated. The resulting residue was purified by silica gel chromatography to provide the title compounds as a mixture (200 mg, yield: 17%). MS (ESI) m/z=210.9 [M+H]⁺.

Step 4. Syntheses of tert-butyl (6-(4-(8-methylquinoxalin-2-yl)-1H-pyrazol-1-yl)hexyl)carbamate and tert-butyl (6-(4-(5-methylquinoxalin-2-yl)-1H-pyrazol-1-yl)hexyl)carbamate

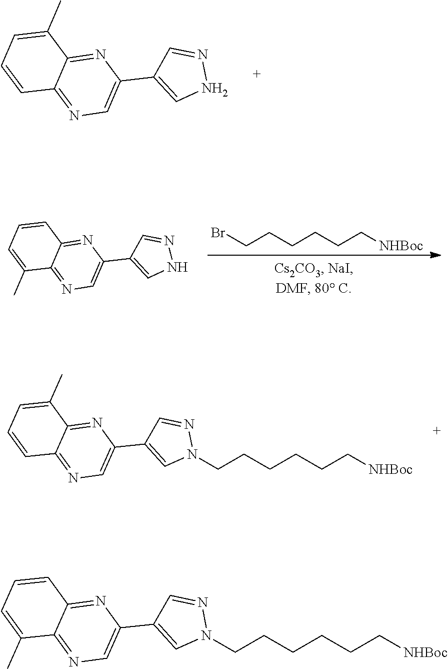 [SMILES: Cc1cccc2ncc(-c3cn[nH]c3)nc12 ; Cc1cccc2nc(-c3cn[nH]c3)cnc12 ; CC(C)(C)OC(=O)NCCCCCCBr ; Cc1cccc2ncc(-c3cnn(CCCCCCNC(=O)OC(C)(C)C)c3)nc12 ; Cc1cccc2nc(-c3cnn(CCCCCCNC(=O)OC(C)(C)C)c3)cnc12]

A solution of methyl-2-(1H-pyrazol-4-yl)quinoxalines (200 mg, 1.32 μmol), tert-buty N-(6-bromohexyl)carbamate (293.22 mg, 1.05 mmol), K₂CO₃ (262.96 mg, 1.90 mmol) in DMF (2 mL) was stirred at 70° C. under N₂ for 2 h. Then, the mixture was concentrated, and the resulting residue was purified by reverse column to provide the title compounds as a mixture (250 mg, yield: 64%). MS (ESI) m/z=410.5 [M+H]⁺.

Step 5. Syntheses of 6-(4-(8-methylquinoxalin-2-yl)-1H-pyrazol-1-yl)hexan-1-amine and 6-(4-(5-methylquinoxalin-2-yl)-1H-pyrazol-1-yl)hexan-1-amine

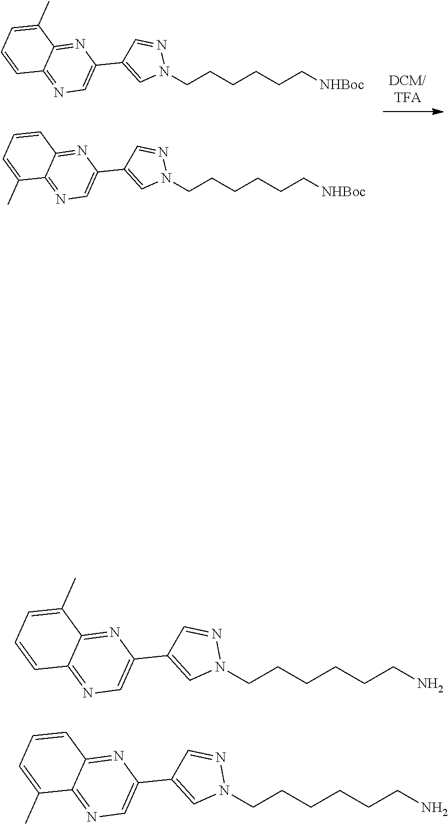 [SMILES: Cc1cccc2ncc(-c3cnn(CCCCCCNC(=O)OC(C)(C)C)c3)nc12 ; Cc1cccc2nc(-c3cnn(CCCCCCNC(=O)OC(C)(C)C)c3)cnc12 ; Cc1cccc2ncc(-c3cnn(CCCCCCN)c3)nc12 ; Cc1cccc2nc(-c3cnn(CCCCCCN)c3)cnc12]

A solution of tert-butyl (6-(4-(methylquinoxalin-2-yl)-1H-pyrazol-1-yl)hexyl)carbamates (110 mg, 268.60 μmol) in DCM (6 mL) and TFA (2 mL) was stirred at rt for 1 h. The mixture was concentrated, and the resulting residue was purified by reverse phase chromatography to provide the title compounds as a mixture (70 mg, yield: 23%). MS (ESI) m/z=310.1 [M+H]⁺.

Step 6. Syntheses of 2-(2,6-dioxopiperidin-3-yl)-5-((6-(4-(8-methylquinoxalin-2-yl)-1H-pyrazol-1-yl)hexyl)amino)isoindoline-1,3-dione and 2-(2,6-dioxopiperidin-3-yl)-5-((6-(4-(5-methylquinoxalin-2-yl)-1H-pyrazol-1-yl)hexyl)amino)isoindoline-1,3-dione

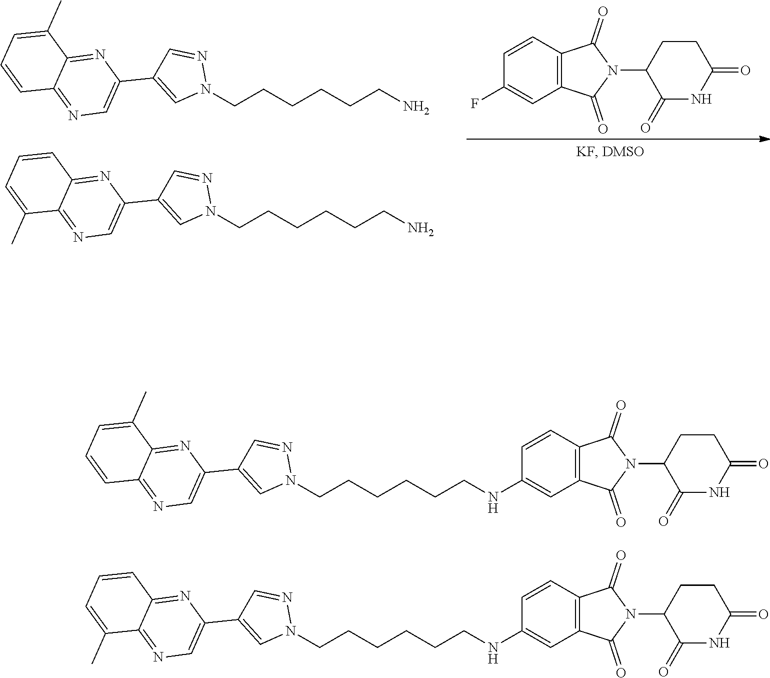

A solution of 6-(4-(methylquinoxalin-2-yl)-1H-pyrazol-1-yl)hexan-1-amines (100 mg, 323.20 μmol), 2-(2,6-dioxo-3-piperidyl)-5-fluoro-isoindoline-1,3-dione (133.91 mg, 484.80 μmol), and KF (56.33 mg, 969.59 μmol) in DMSO (3 mL) was stirred at 130° C. under microwave irradiation for 15 min. The mixture was purified by reverse phase chromatography to provide the title compounds (GS-511, 2.8 mg, yield: 2%, MS (ESI) m/z=566.4 [M+H]⁺; and GS-512, 1.6 mg, yield: 1%, MS (ESI) m/z=566.4 [M+H]⁺).

### Example 471. N-(2-(2,6-Dioxopiperidin-3-yl)-1-oxoisoindolin-5-yl)-3-(4-(4-(quinoxalin-2-yl)-1H-pyrazol-1-yl)piperidin-1-yl)benzamide (GS-513)

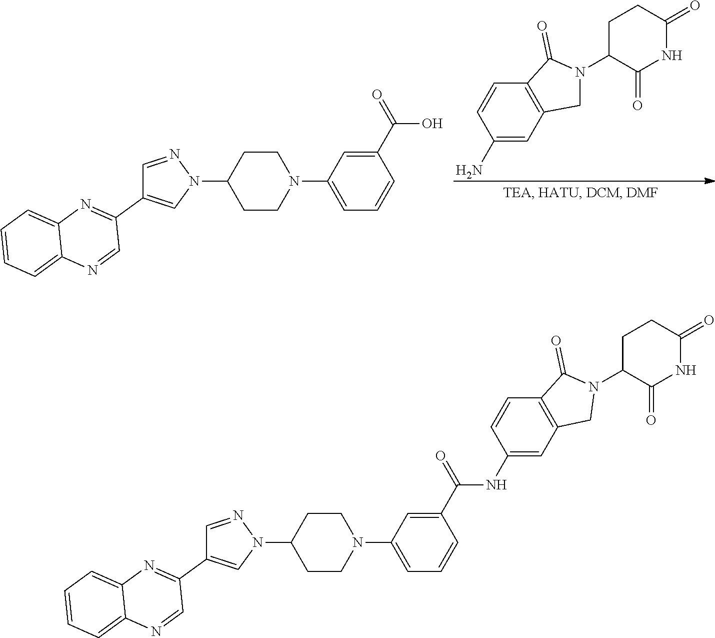 [SMILES: O=C(O)c1cccc(N2CCC(n3cc(-c4cnc5ccccc5n4)cn3)CC2)c1 ; Nc1ccc2c(c1)CN(C1CCC(=O)NC1=O)C2=O ; O=C1CCC(N2Cc3cc(NC(=O)c4cccc(N5CCC(n6cc(-c7cnc8ccccc8n7)cn6)CC5)c4)ccc3C2=O)C(=O)N1]

GS-513 was synthesized following the similar procedures as described for GS-476 (1.5 mg, yield: 5%) as brown solid. MS (ESI) m/z=641.5 [M+H]⁺.

### Example 472. 1-((2-(2,6-Dioxopiperidin-3-yl)-1-oxoisoindolin-5-yl)methyl)-3-(3-(4-phenylpiperidin-1-yl)phenyl)urea (GS-514)

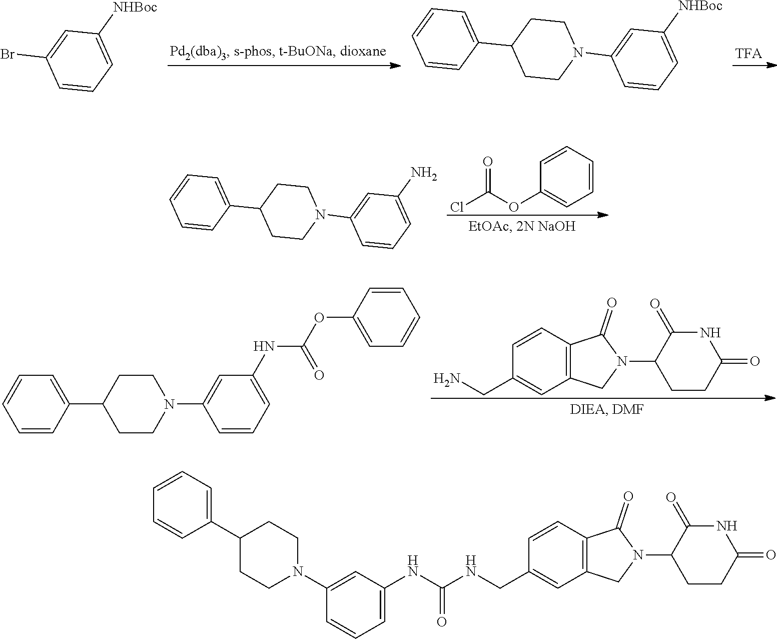

Step 1. Synthesis of tert-butyl (3-(4-phenylpiperidin-1-yl)phenyl)carbamate

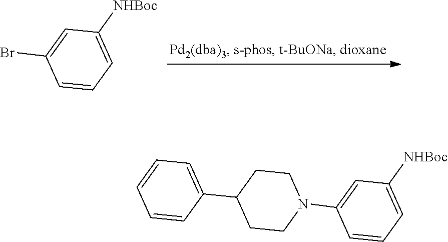 [SMILES: CC(C)(C)OC(=O)Nc1cccc(Br)c1 ; CC(C)(C)OC(=O)Nc1cccc(N2CCC(c3ccccc3)CC2)c1]

To a solution of tert-butyl (3-bromophenyl)carbamate (500 mg, 1.84 mmol) in dioxane (10 mL) were added 4-phenylpiperidine (296 mg, 1.84 mmol), tris(dibenzylideneacetone)dipalladium (82 mg, 0.09 mmol), 2-dicyclohexylphosphino-2′,6′-dimethoxy-1,1′-biphenyl (73 mg, 0.18 mmol) and sodium tert-butoxide (353 mg, 3.68 mmol). The mixture was stirred at 100° C. for 1 h, and purified by reverse phase chromatography (MeOH:H₂O=1:1) to provide the title compound (460 mg, yield: 71%) as yellow solid.

Step 2. Synthesis of 3-(4-phenylpiperidin-1-yl)aniline

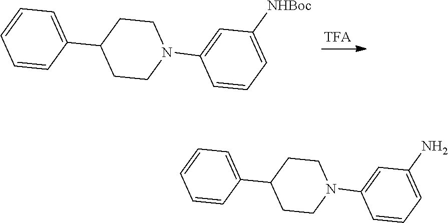 [SMILES: CC(C)(C)OC(=O)Nc1cccc(N2CCC(c3ccccc3)CC2)c1 ; Nc1cccc(N2CCC(c3ccccc3)CC2)c1]

To a solution of tert-butyl (3-(4-phenylpiperidin-1-yl)phenyl)carbamate (460 mg, 1.3 mmol) in DCM (5 mL) was added TFA (2 mL), The mixture was stirred at rt for 1 h, and concentrated to provide the title compound (310 mg, yield: 94%) as yellow oil.

Step 3. Synthesis of phenyl (3-(4-phenylpiperidin-1-yl)phenyl)carbamate

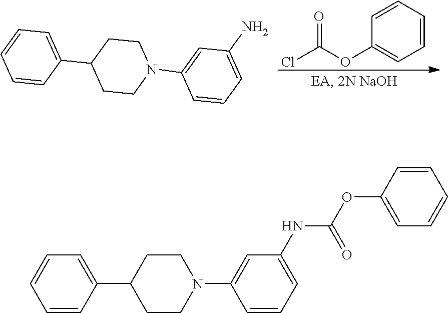 [SMILES: Nc1cccc(N2CCC(c3ccccc3)CC2)c1 ; O=C(Cl)Oc1ccccc1 ; O=C(Nc1cccc(N2CCC(c3ccccc3)CC2)c1)Oc1ccccc1]

To a solution of 3-(4-phenylpiperidin-1-yl)aniline (50 mg, 0.2 mmol) in EtOAc (5 mL) were added aq. NaOH solution (2 M, 3 mL) and phenyl carbonochloridate (62 mg, 0.4 mmol). The mixture was stirred at rt for 2 h, and then purified by column chromatography (petroleum ether:EtOAc=10:1) to provide the title compound (42 mg, yield: 57%) as colorless oil. MS (ESI) m/z=373.4 [M+H]⁺.

Step 4. Synthesis of 1-((2-(2,6-dioxopiperidin-3-yl)-1-oxoisoindolin-5-yl)methyl)-3-(3-(4-phenylpiperidin-1-yl)phenyl)urea

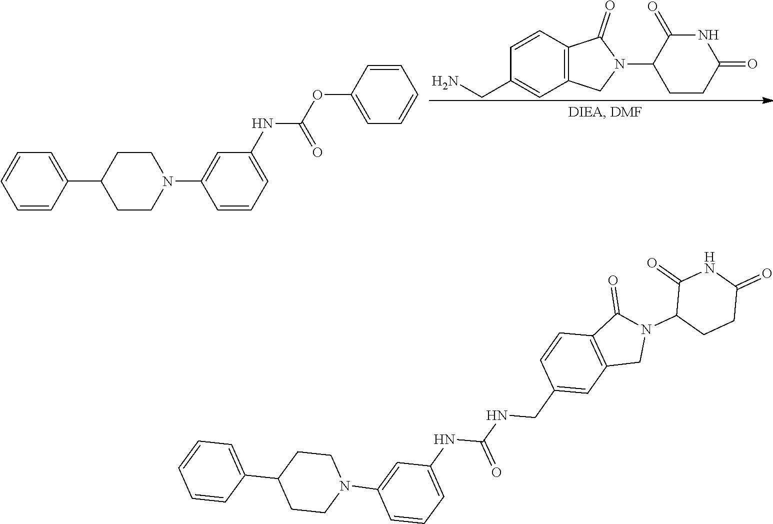

To a solution of phenyl (3-(4-phenylpiperidin-1-yl)phenyl)carbamate (42 mg, 0.11 mmol) in DMF (1 mL) were added 3-(5-(aminomethyl)-1-oxoisoindolin-2-yl)piperidine-2,6-dione (30 mg, 0.11 mmol) and DIEA (42 mg, 0.33 mmol). The mixture was stirred at rt for 16 h, and then purified by reverse phase chromatography (MeOH:H₂O=1:1) to provide the title compound (10 mg, yield: 16%) as white solid. MS (ESI) m/z=552.4 [M+H]⁺.

### Example 473. 3-(1-Oxo-5-((5-oxo-5-(4-(4-(quinoxalin-2-yl)-1H-pyrazol-1-yl)piperidin-1-yl)pentyl)amino)isoindolin-2-yl)piperidine-2,6-dione (GS-515)

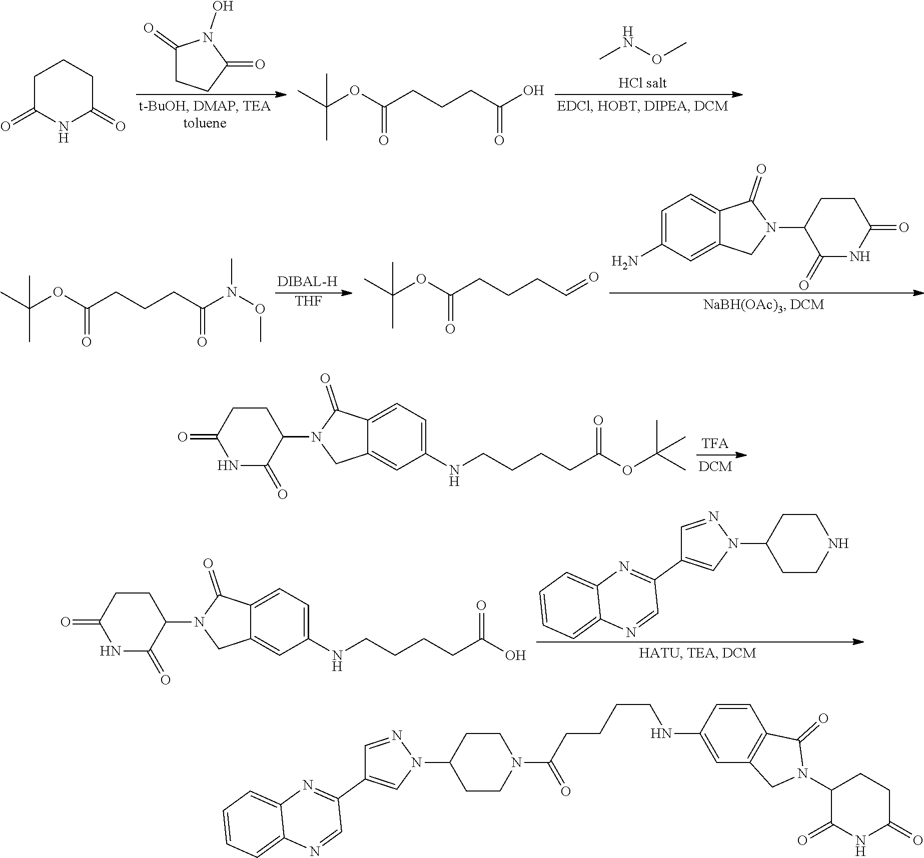

Step 1. Synthesis of 5-tert-butoxy-5-oxo-pentanoic acid

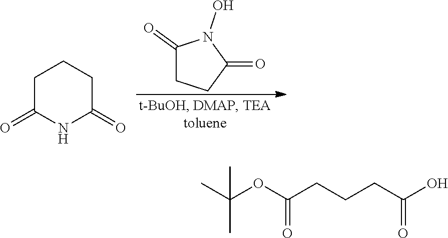

A mixture of tetrahydropyran-2,6-dione (5 g, 43.82 mmol), 2-methylpropan-2-ol (9.74 g, 131.46 mmol), 2-methylpropan-2-ol (9.74 g, 131.46 mmol), TEA (1.33 g, 13.15 mmol) and DMAP (535.35 mg, 4.38 mmol) in toluene (50 mL) was stirred at 115° C. for 24 h. The mixture was cooled to rt, and diluted with EtOAc (200 mL). The mixture was washed with aq. HCl (5%, 30 mL×3). The organic layer were dried over with Na₂SO₄, filtered, and concentrated in vacuo to provide the title compound (7 g, yield: 85%) as colorless oil.

Step 2. Synthesis of tert-butyl 5-(methoxy(methyl)amino)-5-oxopentanoate

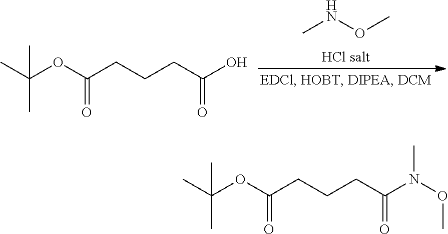

To a solution of 5-tert-butoxy-5-oxo-pentanoic acid (500 mg, 2.66 mmol) were added N-methoxymethanamine (HCl salt, 285.03 mg, 2.92 mmol), EDCI (191.7 g, 2.92 mmol), HOBT (394.84 mg, 2.92 mmol) and DIPEA (858.32 mg, 6.64 mmol) at rt. The mixture was stirred for 4 h, and then quenched with water (1 mL). The mixture was concentrated in vavuo to remove most of DCM. The resulting residue was dissolved in EtOAc (50 mL) and washed with water (30 mL). The organic layer was dried over Na₂SO₄, filtered, and concentrated in vacuo to provide the title compound (600 mg, yield: 98%) as yellow oil.

Step 3. Synthesis of tert-butyl 5-oxopentanoate

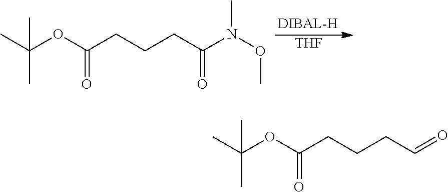

To a solution of tert-butyl 5-(methoxy(methyl)amino)-5-oxopentanoate (0.2 g, 864.72 μmol) in anhydrous THF (5 mL) was added DIBAl-H (1 M in toulene, 1.73 ml, 1.73 mmol) at 0° C. The mixture was stirred at 0° C. for 0.5 h. The mixture was quenched with water (5 mL), and extracted with EtOAc (10 mL). The organic layer was dried over Na₂SO₄, filtered, and concentrated in vacuo to provide the title compound (148 mg, yield: 99%) as colorless oil.

Step 4. Synthesis of tert-butyl 5-((2-(2,6-dioxopiperidin-3-yl)-1-oxoisoindolin-5-yl)amino)pentanoate

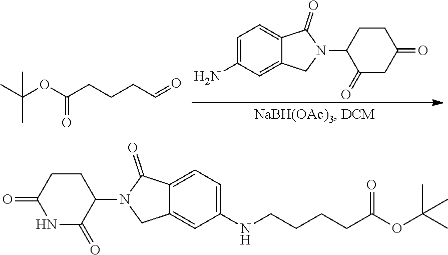 [SMILES: CC(C)(C)OC(=O)CCCC=O ; Nc1ccc2c(c1)CN(C1CCC(=O)CC1=O)C2=O ; CC(C)(C)OC(=O)CCCCNc1ccc2c(c1)CN(C1CCC(=O)NC1=O)C2=O]

To a mixture of 3-(5-amino-1-oxo-isoindolin-2-yl)piperidine-2,6-dione (200 mg, 771.43 μmol) and tert-butyl 5-oxopentanoate (146.14 mg, 848.57 μmol) in DCM (8 mL) was added AcOH (60.05 g, 385.71 μmol) at 25° C. The mixture was stirred at 25° C. for 2 h. To the mixture was added NaBH(OAc)₃ (326.96 mg, 1.54 mmol) at 25° C. The obtained mixture was stirred for 6 h. After concentration, The resulting residue was purified by flash chromatography to provide the title compound (25 mg, yield: 8%) as brown solid. MS (ESI) m/z=416.3 [M+H]⁺.

Step 5. Synthesis of 5-((2-(2,6-dioxopiperidin-3-yl)-1-oxoisoindolin-5-yl)amino)pentanoic acid

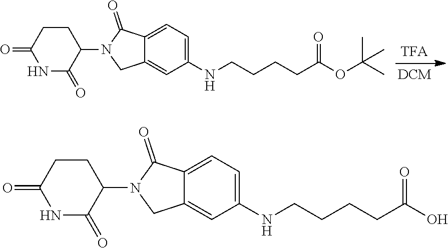 [SMILES: CC(C)(C)OC(=O)CCCCNc1ccc2c(c1)CN(C1CCC(=O)NC1=O)C2=O ; O=C(O)CCCCNc1ccc2c(c1)CN(C1CCC(=O)NC1=O)C2=O]

To a solution of tert-butyl 5-((2-(2,6-dioxopiperidin-3-yl)-1-oxoisoindolin-5-yl)amino)pentanoate (25 mg, 60.17 μmol) in DCM (2 mL) was added TFA (1 mL) at 25° C. After the reaction was stirred for 2 h, the mixture was concentrated in vacuo to provide the title compound (20 mg, yield: 92%) as brown solid. MS (ESI) m/z=360.1 [M+H]⁺.

Step 6. Synthesis of 3-(1-oxo-5-((5-oxo-5-(4-(4-(quinoxalin-2-yl)-1H-pyrazol-1-yl)piperidin-1-yl)pentyl)amino)isoindolin-2-yl)piperidine-2,6-dione

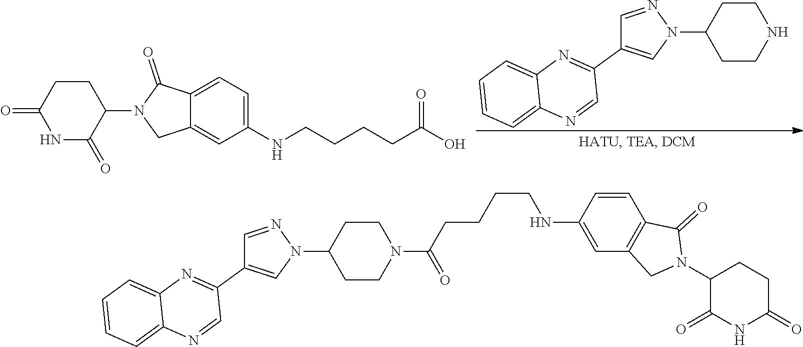

To a mixture of 2-(1-(piperidin-4-yl)-1H-pyrazol-4-yl)quinoxaline (15.55 mg, 55.65 μmol) and 5-((2-(2,6-dioxopiperidin-3-yl)-1-oxoisoindolin-5-yl)amino)pentanoic acid (20 mg, 55.65 μmol) in DCM (3 mL) were added TEA (16.89 mg, 166.96 pml) and HATU (31.74 mg, 83.48 μmol) at 25° C. The mixture was stirred for 2 h, and then quenched with water (0.2 mL). The mixture was concentrated in vacuo, and purified by flash chromatography to provide the title compound (3 mg, yield: 9%) as white solid. MS (ESI) m/z=621.5 [M+H]⁺.

### Example 474. 2-(2,6-Dioxopiperidin-3-yl)-5-((6-(4-(o-tolyl)-1H-pyrazol-1-yl)hexyl)amino)isoindoline-1,3-dione (GS-516)

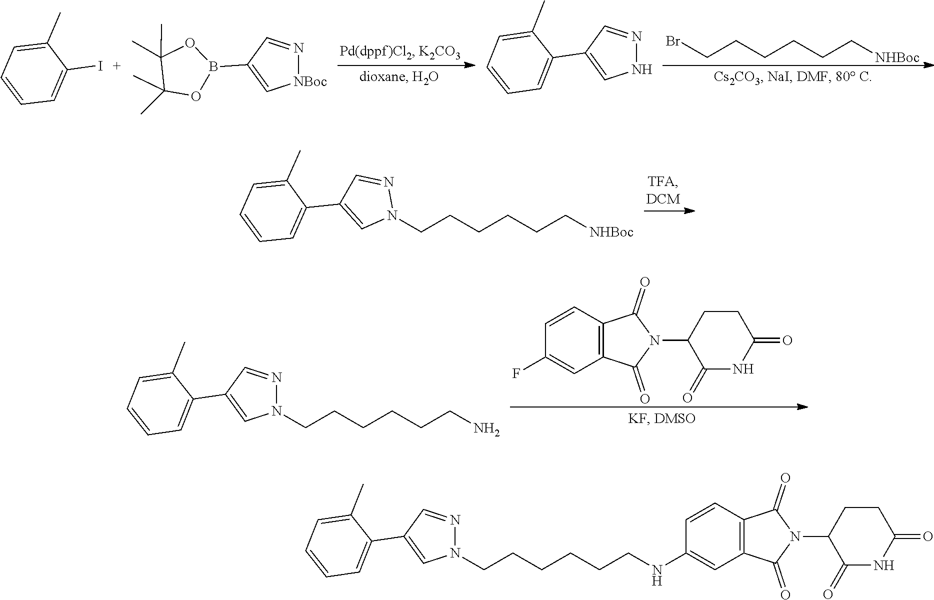

GS-516 was synthesized following the similar procedures as described for GS-498 (2.3 mg). MS (ESI) m/z=514.3 [M+H]⁺.

### Example 475. 5-((6-(4-(6-Bromoquinoxalin-2-yl)-1H-pyrazol-1-yl)hexyl)amino)-2-(2,6-dioxopiperidin-3-yl)isoindoline-1,3-dione (GS-517)

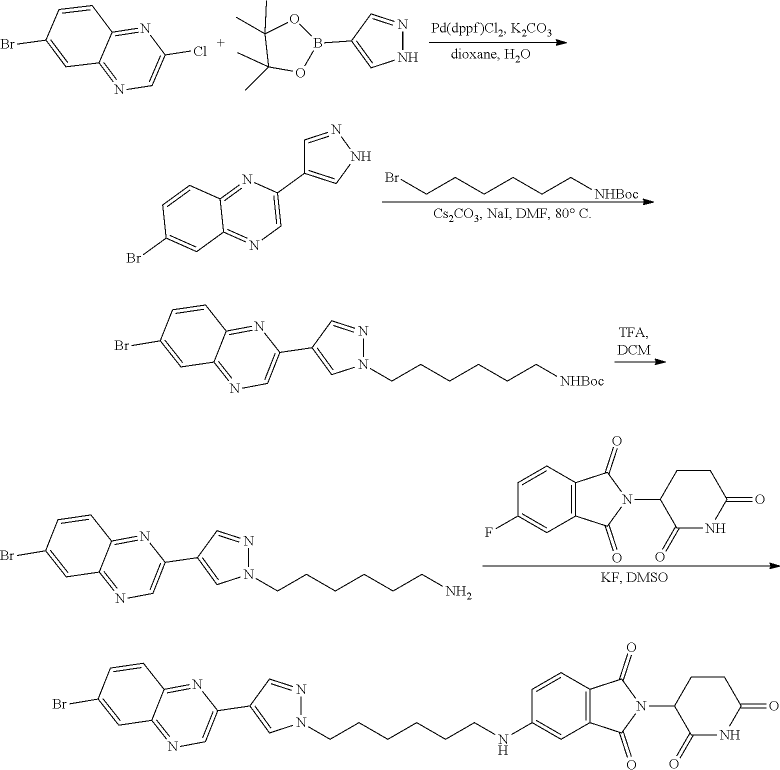

GS-517 was synthesized following the similar procedures as described for GS-521 (2.3 mg). MS (ESI) m/z=632.2 [M+H]⁺.

### Example 476. N-((2-(2,6-Dioxopiperidin-3-yl)-1-oxoisoindolin-5-yl)methyl)-4,4-difluoro-5-oxo-5-(4-(4-(quinoxalin-2-yl)-1H-pyrazol-1-yl)piperidin-1-yl)pentanamide (GS-518)

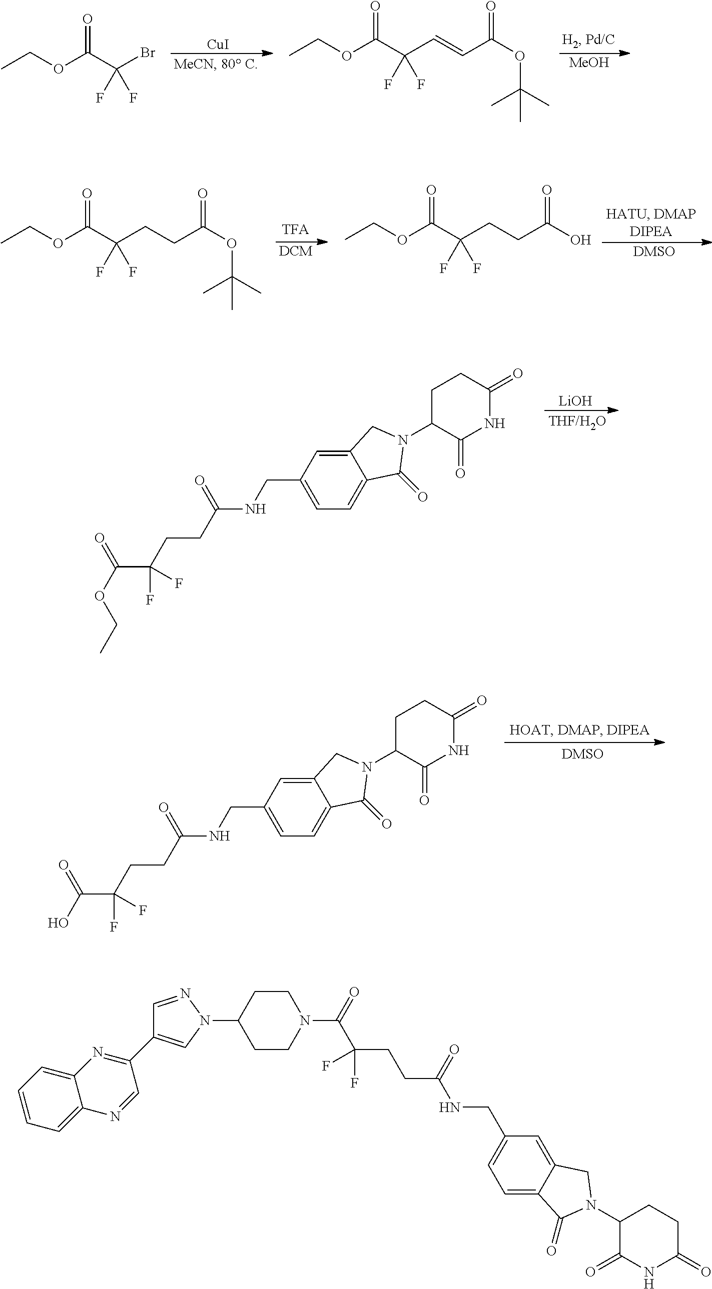

Step 1. Synthesis of 1-(tert-butyl) 5-ethyl (E)-4,4-difluoropent-2-enedioate

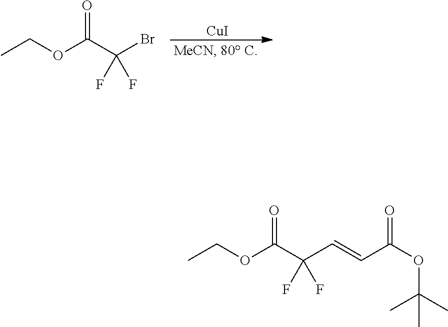

To a mixture of ethyl 2-bromo-2,2-difluoro-acetate (5.0 g, 24 mmol) and tert-butyl prop-2-enoate (4.7 g, 36 mmol) in MeCN (50 mL) were added CuI (469 mg, 2.4 mmol) and pentamethyldiethylenetriamine (6.4 g, 36.9 mmol). The mixture was purged with N₂ for three times. After being stirred at 80° C. overnight, the mixture was quenched with water, and extracted with EtOAc. The organic layer was washed with brine, dried over Na₂SO₄, filtered, and concentrated. The resulting residue was purified by flash chromatography (petroleum ether:EtOAc=1:0 to 7:3) to provide the title compound (2.2 g, yield: 35%) as bright oil. ¹H NMR (400 MHz, CDCl₃) δ 6.79 (dt, J=15.8, 11.5 Hz, 1H), 6.36 (dt, J=15.8, 2.3 Hz, 1H), 4.35 (q, J=7.1 Hz, 2H), 1.51 (s, 9H), 1.37 (t, J=7.2 Hz, 3H).

Step 2. Synthesis of 5-(tert-butyl) 1-ethyl 2,2-difluoropentanedioate

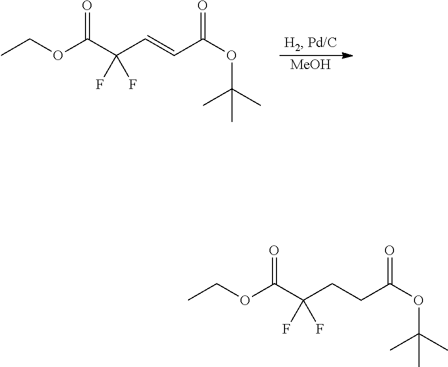 [SMILES: "MeOH" ; CCOC(=O)C(F)(F)/C=C/C(=O)OC(C)(C)C ; CCOC(=O)C(F)(F)CCC(=O)OC(C)(C)C]

To a solution of 1-(tert-butyl) 5-ethyl (E)-4,4-difluoropent-2-enedioate (2.2 g, 8.7 mmol) in methanol (40 mL) was added Pd/C (209 mg, 1.7 mmol). The mixture was purged with H₂ for three times. After stirred at rt for 2 h, the mixture was filtered, and concentrated to provide the title compound (2.2 g, yield: 99%) as bright oil. ¹H NMR (400 MHz, CDCl₃) δ 4.33 (q, J=7.2 Hz, 2H), 2.52-2.30 (m, 4H), 1.45 (s, 5H), 1.36 (t, J=7.2 Hz, 2H); MS (ESI) m/z=253.2 [M+H]⁺.

Step 3. Synthesis of 5-ethoxy-4,4-difluoro-5-oxopentanoic acid

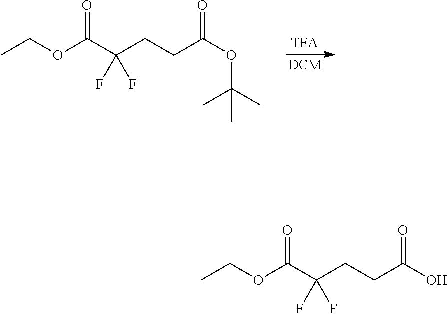

To a solution of 5-(tert-butyl) 1-ethyl 2,2-difluoropentanedioate (1.1 g, 4.3 mmol) in DCM (5 mL) was added TFA (5 mL). The reaction was stirred at rt for 7 h. The mixture was concentrated to provide the title compound (800 mg, yield: 93%), which was directly used for next step. MS (ESI) m/z=194.9 [M−H]⁻.

Step 4. Synthesis of ethyl 5-(((2-(2,6-dioxopiperidin-3-yl)-1-oxoisoindolin-5-yl)methyl)amino)-2,2-difluoro-5-oxopentanoate

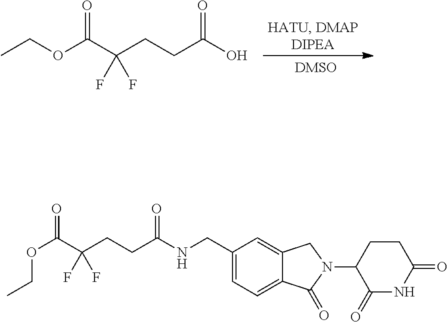 [SMILES: CCOC(=O)C(F)(F)CCC(=O)O ; CCOC(=O)C(F)(F)CCC(=O)NCc1ccc2c(c1)CN(C1CCC(=O)NC1=O)C2=O]

To a mixture of 3-(5-(aminomethyl)-1-oxoisoindolin-2-yl)piperidine-2,6-dione (150 mg, 548 μmol) and 5-ethoxy-4,4-difluoro-5-oxo-pentanoic acid (161 mg, 823 μmol) in DMSO (5 mL) was added HATU (417 mg, 1.1 mmol), DMAP (6.7 mg, 54 μmol) and DIPEA (283 mg, 2.2 mmol). The mixture was stirred at rt overnight. The reaction was purified by reverse phase chromatography (0-70% MeOH in H₂O) to provide the title compound (150 mg, yield: 60%) as white solid. MS (ESI) m/z=452.1 [M+H]⁺.

Step 5. Synthesis of 5-(((2-(2,6-dioxopiperidin-3-yl)-1-oxoisoindolin-5-yl)methyl)amino)-2,2-difluoro-5-oxopentanoic acid

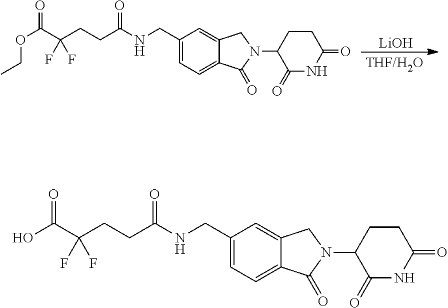

To a solution of ethyl 5-(((2-(2,6-dioxopiperidin-3-yl)-1-oxoisoindolin-5-yl)methyl)amino)-2,2-difluoro-5-oxopentanoate (150 mg, 332 μmol) in water (1 mL) and THF (4 mL) was added LiOH (7.9 mg, 332 μmol) at ice/salt bath. The reaction mixture was stirred at rt for 2 h, and then purified by reverse phase chromatography (0-70% MeOH in H₂O) to provide the title compound (70 mg, yield: 50%) as bright oil. MS (ESI) m/z=424.3 [M+H]⁺.

Step 6. Synthesis of N-((2-(2,6-dioxopiperidin-3-yl)-1-oxoisoindolin-5-yl)methyl)-4,4-difluoro-5-oxo-5-(4-(4-(quinoxalin-2-yl)-1H-pyrazol-1-yl)piperidin-1-yl)pentanamide

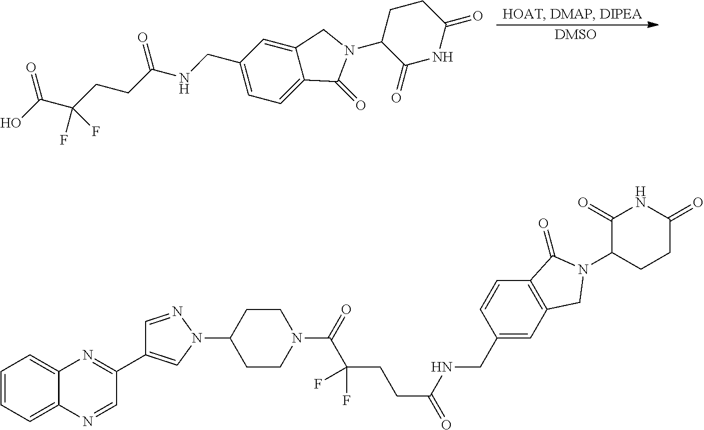 [SMILES: O=C(CCC(F)(F)C(=O)O)NCc1ccc2c(c1)CN(C1CCC(=O)NC1=O)C2=O ; O=C(CCC(F)(F)C(=O)N1CCC(n2cc(-c3cnc4ccccc4n3)cn2)CC1)NCc1ccc2c(c1)CN(C1CCC(=O)NC1=O)C2=O]

To a mixture of 5-(((2-(2,6-dioxopiperidin-3-yl)-1-oxoisoindolin-5-yl)methyl)amino)-2,2-difluoro-5-oxopentanoic acid (70 mg, 165 μmol) and 2-(1-(piperidin-4-yl)-1H-pyrazol-4-yl)quinoxaline (69 mg, 248 μmol) in DMSO (4 mL) were added HATU (8 mg, 330 μmol) and DIPEA (64 mg, 496 μmol). The mixture was stirred at rt overnight. The reaction was purified by reverse phase chromatography (0-70% MeCN in H₂O) to provide the title compound (28.5 mg, yield: 25%) as yellow solid. MS (ESI) m/z=685.4 [M+H]⁺.

### Example 477. 3-(1-Oxo-6-((5-(4-(quinoxalin-2-yl)-1H-pyrazol-1-yl)pentyl)amino)isoindolin-2-yl)piperidine-2,6-dione (GS-519)

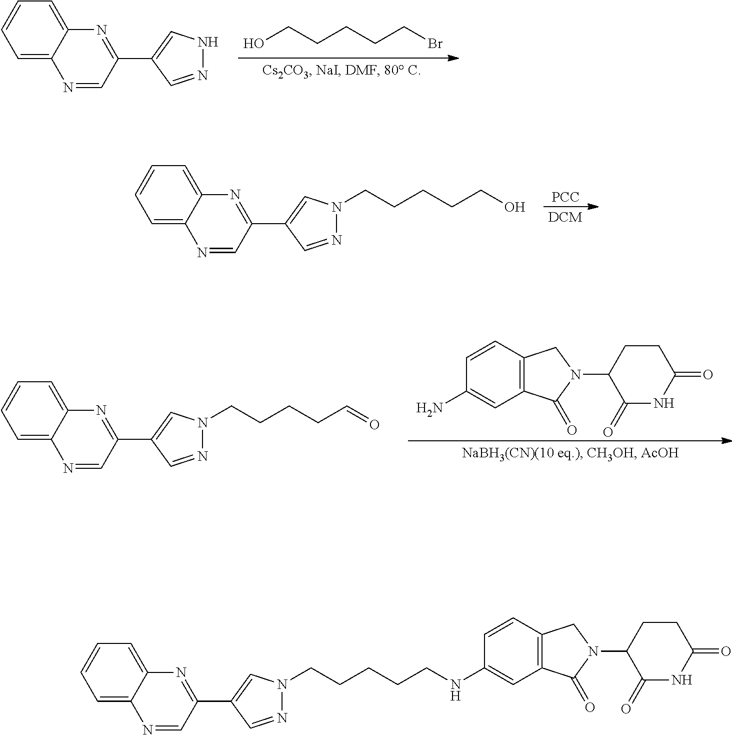

Step 1. Synthesis of 5-(4-(quinoxalin-2-yl)-1H-pyrazol-1-yl)pentan-1-ol

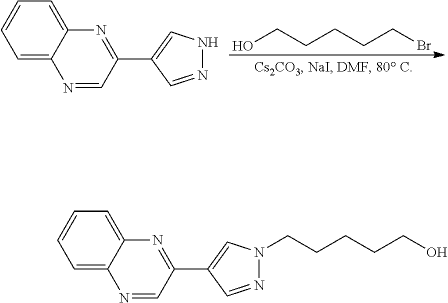

To a solution of 2-(1H-pyrazol-4-yl)quinoxaline (80 mg, 0.41 mmol) in DMF (2 mL) were added 5-bromopentan-1-ol (68 mg, 0.41 mmol), Cs₂CO₃ (266 mg, 0.82 mmol) and NaI (65 mg, 0.41 mmol). The mixture was stirred at 100° C. for 1 h. The reaction mixture was purified by reverse phase chromatography (MeOH:H₂O=2:1) to provide the title compound (106 mg, yield: 92%) as yellow oil.

Step 2. Synthesis of 5-(4-(quinoxalin-2-yl)-1H-pyrazol-1-yl)pentanal

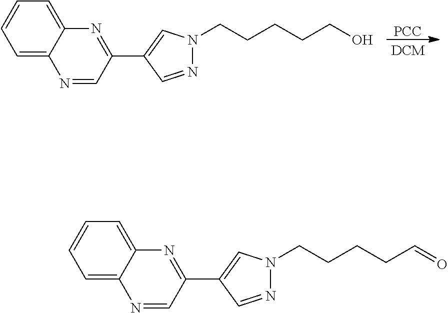

To a solution of 5-(4-(quinoxalin-2-yl)-1H-pyrazol-1-yl)pentan-1-ol (106 mg, 0.37 mmol) in DCM (5 mL) was added PCC (159 mg, 0.74 mmol). The mixture was stirred at rt for 16 h, and then concentrated. The resulting residue was purified by reverse phase chromatography (MeOH:H₂O=2:1) to provide the title compound (86 mg, yield: 82%) as yellow oil.

Step 3. Synthesis of 3-(1-oxo-6-((5-(4-(quinoxalin-2-yl)-1H-pyrazol-1-yl)pentyl)amino)isoindolin-2-yl)piperidine-2,6-dione

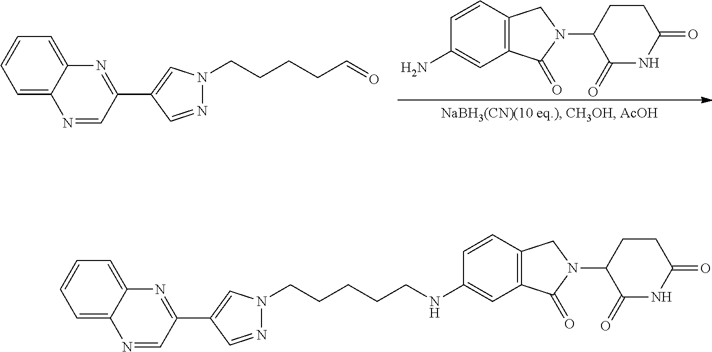 [SMILES: O=CCCCCn1cc(-c2cnc3ccccc3n2)cn1 ; Nc1ccc2c(c1)C(=O)N(C1CCC(=O)NC1=O)C2 ; O=C1CCC(N2Cc3ccc(NCCCCCn4cc(-c5cnc6ccccc6n5)cn4)cc3C2=O)C(=O)N1]

To a solution of 5-(4-(quinoxalin-2-yl)-1H-pyrazol-1-yl)pentanal (20 mg, 0.07 mmol) in methanol (3 mL) were added NaBH₃CN (43 mg, 0.7 mmol) and AcOH (0.05 mL). The mixture was stirred at rt for 2 h, and then purified by reverse phase chromatography (MeOH:H₂O=1:1) to provide the title compound (15 mg, yield: 40%) as white solid. MS (ESI) m/z=524.4 [M+H]⁺.

### Example 478. N-((2-(2,6-Dioxopiperidin-3-yl)-1-oxoisoindolin-5-yl)methyl)-4,4-difluoro-5-oxo-5-(4-(4-(quinoxalin-2-yl)-1H-pyrazol-1-yl)piperidin-1-yl)pentanamide (GS-520)

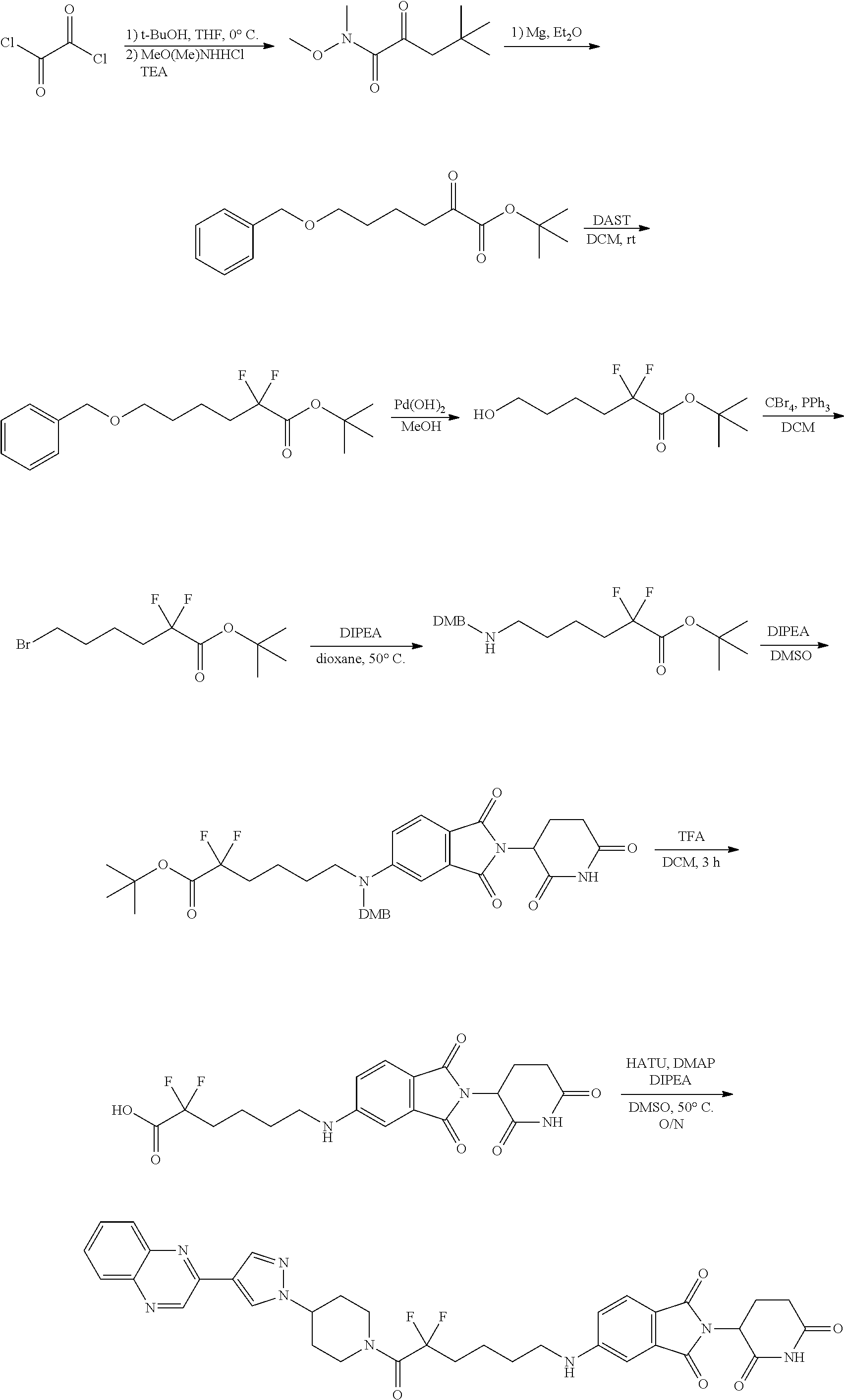

Step 1. Synthesis of tert-butyl 2-(methoxy(methyl)amino)-2-oxoacetate

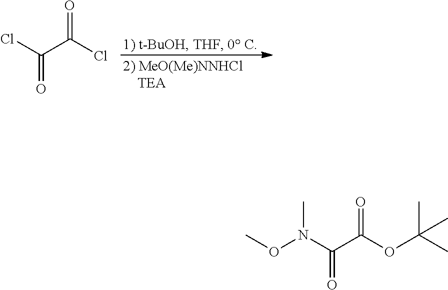

To a solution of oxalyl dichloride (7.5 g, 59 mmol) in THF (90 mL) was added t-BuOH (4.3 g, 59 mmol) at 0° C. After the mixture was stirred for 1 h, N-methoxymethanamine hydrochloride (5.7 g, 59 mmol) and TEA (17.9 g, 177 mmol) were added. The obtained mixture was stirred at 0° C. for 2 h, before it was quenched with water, and extracted with EtOAc. The organic layer was washed with brine, dried over Na₂SO₄, filtered, and concentrated. The resulting residue was purified by silica gel chromatography (petroleum ether:EtOAc=1:0 to 3:2) to provide the title compound (8 g, yield: 71%) as bright solid. ¹H NMR (400 MHz, CDCl₃) δ 3.75 (s, 3H), 3.21 (s, 3H), 1.55 (d, J=8.0 Hz, 10H).

Step 2. Synthesis of tert-butyl 6-(benzyloxy)-2-oxohexanoate

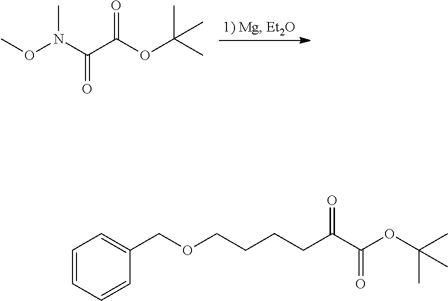 [SMILES: CON(C)C(=O)C(=O)OC(C)(C)C ; CC(C)(C)OC(=O)C(=O)CCCCOCc1ccccc1]

To a solution of I₂ (100 mg, 393 μmol) in Et₂O (4 mL) was added Mg (299 mg, 12 mmol) at rt. The reaction was stirred until the yellow color was faded out. A solution of 4-bromobutoxymethylbenzene (1.5 g, 6.1 mmol) in Et₂O (4 mL) at 0° C. was added. The mixture was stirred for 1 h, before it was cooled to −70° C. A solution of tert-butyl 2-(methoxy(methyl)amino)-2-oxoacetate (1.7 g, 9 mmol) in DCM (4 mL) was added. The reaction mixture was stirred at −70° C. for 1.5 h, and then quenched with aq. NH₄Cl solution. EtOAc and celite were added. The mixture was filtered, and the filtrate was concentrated. The resulting residue was purified by flash chromatography (petroleum ether:EtOAc=1:0 to 3:2) to provide the title compound (1.0 g, yield: 55%) as bright solid. ¹H NMR (400 MHz, CDCl₃) δ 7.46-7.27 (m, 4H), 4.49 (s, 2H), 3.48 (t, J=6.1 Hz, 2H), 2.81 (t, J=7.1 Hz, 2H), 1.81-1.61 (m, 4H), 1.54 (s, 9H).

Step 3. Synthesis of tert-butyl 6-(benzyloxy)-2,2-difluorohexanoate

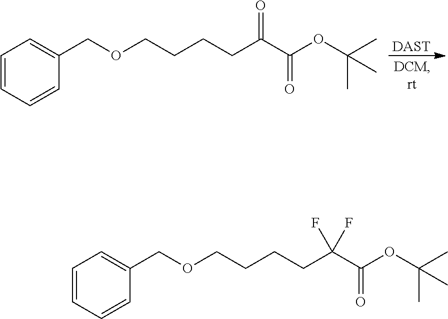

To a solution of tert-butyl 6-benzyloxy-2-oxo-hexanoate (1.0 g, 3.4 mmol) in DCM (15 mL) was added DAST (2.7 g, 17 mmol) at 0° C. The mixture was stirred for 2 h, and then quenched with aq. NaHCO₃ solution. The obtained mixture was extracted with DCM. The organic layer was washed with brine, dried over Na₂SO₄, filtered, and concentrated. The resulting residue was purified by flash chromatography (petroleum ether:EtOAc=1:0 to 2:3) to provide the title compound (700 mg, yield: 65%) as bright oil.

Step 4. Synthesis of tert-butyl 2,2-difluoro-6-hydroxyhexanoate

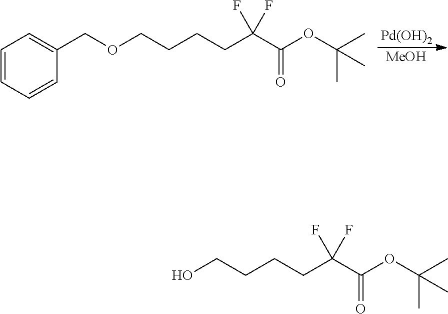

To a solution of tert-butyl 6-benzyloxy-2,2-difluoro-hexanoate (700 mg, 2.2 mmol) in methanol (15 mL) was added Pd(OH)₂ (222 ug, 2.2 mmol). The reaction mixture was stirred at 45° C. overnight under H₂. The mixture was filtered and concentrated to provide the title compound (490 mg, yield: 98%) as bright oil.

Step 5. Synthesis of tert-butyl 6-bromo-2,2-difluorohexanoate

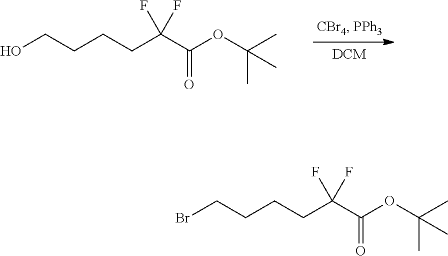

To a solution of tert-butyl 2,2-difluoro-6-hydroxy-hexanoate (500 mg, 2.2 mmol) in DCM (10 mL) were added CBr₄ (1.4 g, 4.4 mmol) and PPh₃ (876 mg, 3.3 mmol). The mixture was stirred at rt for 1 h. The solution was quenched with water, and extracted with DCM. The organic layer was washed with brine, dried over Na₂SO₄, filtered, and concentrated. The resulting residue was purified by silica gel chromatography (petroleum ether:EtOAc=1:0 to 3:2) to provide the title compound (550 mg, yield: 86%) as bright oil.

Step 6. Synthesis of tert-butyl 6-((3,4-dimethylbenzyl)amino)-2,2-difluorohexanoate

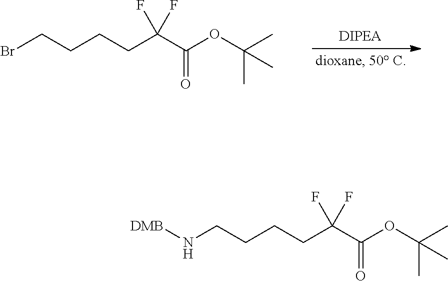

To a solution of tert-butyl 6-bromo-2,2-difluoro-hexanoate (550 mg, 1.9 mmol) in dioxane (10 mL) were added (2,4-dimethoxyphenyl)methanamine (640 mg, 3.8 mmol) and DIPEA (742 mg, 5.7 mmol). The reaction was stirred at 50° C. overnight, and then quenched with water. The obtained mixture was extracted with DCM. The organic layer was washed with brine, dried over Na₂SO₄, filtered, and concentrated. The resulting residue was purified by silica gel chromatography (petroleum ether:EtOAc=1:0 to 3:2) to provide the title compound (300 mg, yield: 42%) as yellow oil. MS (ESI) m/z=374.3 [M+H]⁺.

Step 7. Synthesis of tert-butyl 6-((3,4-dimethoxybenzyl)(2-(2,6-dioxopiperidin-3-yl)-1,3-dioxoisoindolin-5-yl)amino)-2,2-difluorohexanoate

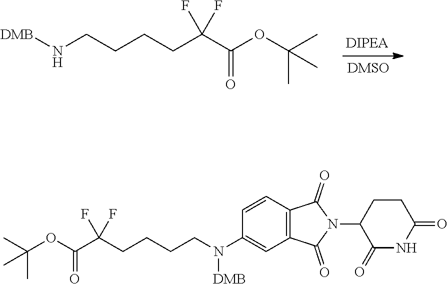

To a mixture of tert-butyl 6-((3,4-dimethylbenzyl)amino)-2,2-difluorohexanoate (250 mg, 732 μmol) and 2-(2,6-dioxo-3-piperidyl)-5-fluoro-isoindoline-1,3-dione (242 mg, 878 μmol) in DMSO (10 mL) was added DIPEA (283 mg, 2.2 mmol). The mixture was stirred at 100° C. for 5 h. The solution was quenched with water, and extracted with EtOAc. The organic layer was washed with brine, dried over Na₂SO₄, filtered, and concentrated. The resulting residue was purified by reverse phase chromatography (0-70% MeCN in H₂O) to provide the title compound (45 mg, yield: 10%) as bright oil. MS (ESI) m/z=630.4 [M+H]⁺.

Step 8. Synthesis of 6-((2-(2,6-dioxopiperidin-3-yl)-1,3-dioxoisoindolin-5-yl)amino)-2,2-difluorohexanoic acid

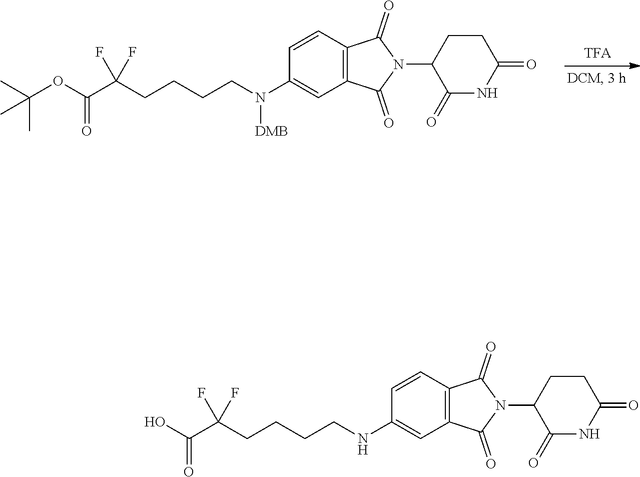

A solution of tert-butyl 6-((3,4-dimethylbenzyl)(2-(2,6-dioxopiperidin-3-yl)-1,3-dioxoisoindolin-5-yl)amino)-2,2-difluorohexanoate (45 mg, 75 μmol) in DCM (4 mL) and TFA (2 mL) was stirred at rt for 3 h. After the reaction was concentrated, it was purified by reverse phase chromatography (0-70% MeCN in H₂O) to provide the title compound (25 mg, yield: 78%) as bright oil. MS (ESI) m/z=424.2 [M+H]⁺.

Step 9. Synthesis of 5-((5,5-difluoro-6-oxo-6-(4-(4-(quinoxalin-2-yl)-1H-pyrazol-1-yl)piperidin-1-yl)hexyl)amino)-2-(2,6-dioxopiperidin-3-yl)isoindoline-1,3-dione

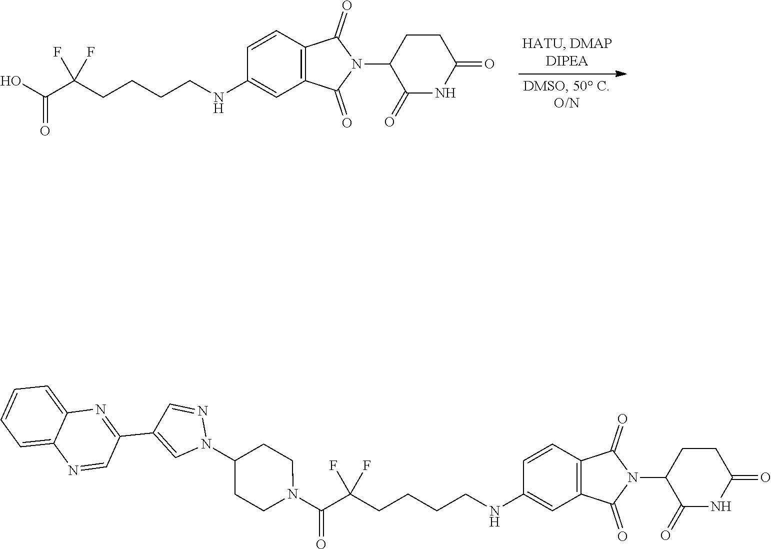 [SMILES: O=C1CCC(N2C(=O)c3ccc(NCCCCC(F)(F)C(=O)O)cc3C2=O)C(=O)N1 ; O=C1CCC(N2C(=O)c3ccc(NCCCCC(F)(F)C(=O)N4CCC(n5cc(-c6cnc7ccccc7n6)cn5)CC4)cc3C2=O)C(=O)N1]

To a mixture of 6-((2-(2,6-dioxopiperidin-3-yl)-1,3-dioxoisoindolin-5-yl)amino)-2,2-difluorohexanoic acid (20 mg, 47 μmol) and 2-(1-(piperidin-4-yl)-1H-pyrazol-4-yl)quinoxaline (see step 2 of GS-005 for synthesis, 15 mg, 56 μmol) in DMSO (5 mL) were added HATU (35 mg, 94 μmol), DMAP (577 ug, 4.7 μmol) and DIPEA (18 mg, 141 μmol). The reaction mixture was stirred at 50° C. overnight. The obtained solution was purified by reverse phase chromatography (0-70% MeCN in H₂O) to provide the title compound (2.9 mg, yield: 9%) as yellow solid. MS (ESI) m/z=685.5 [M+H]⁺.

### Example 479. 5-((6-(4-(3-Cyclopropylquinoxalin-2-yl)-1H-pyrazol-1-yl)hexyl)amino)-2-(2,6-dioxopiperidin-3-yl)isoindoline-1,3-dione (GS-521)

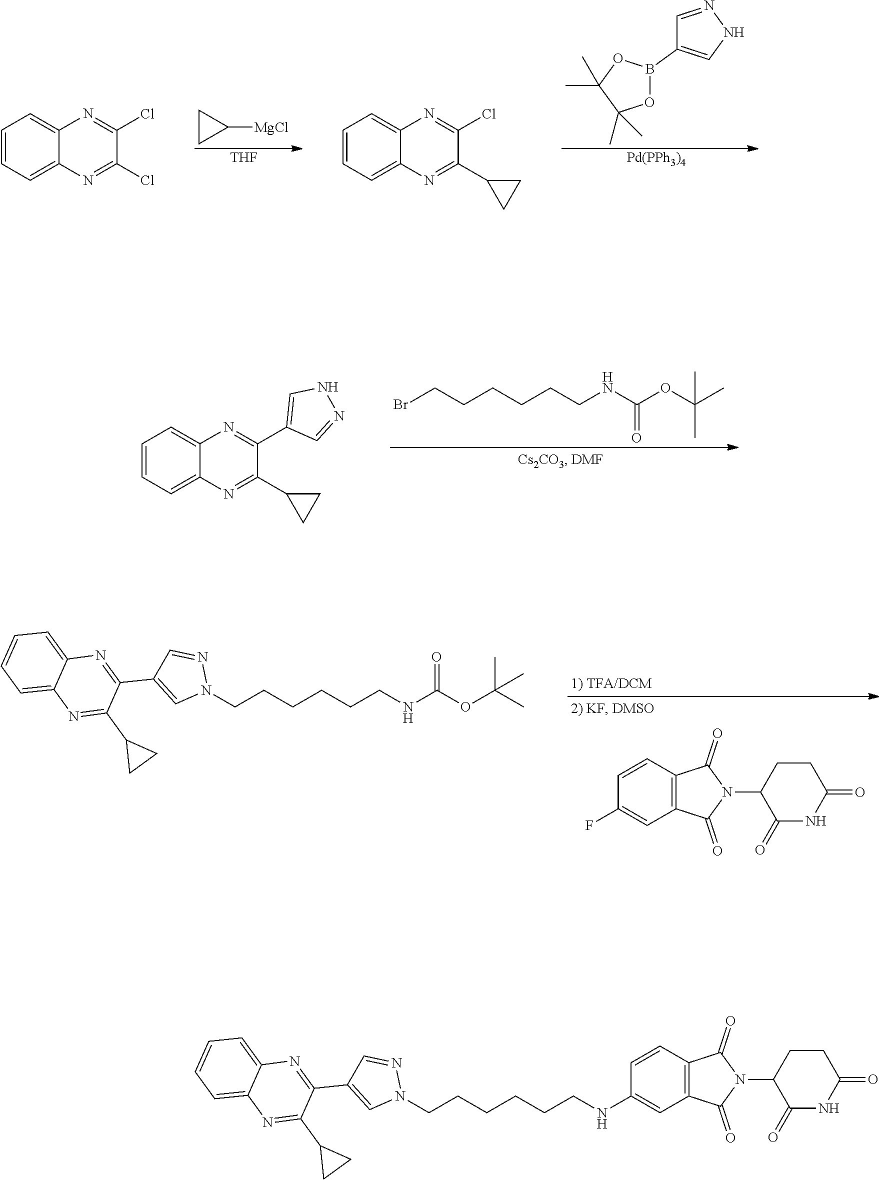

Step 1. Synthesis of 2-chloro-3-cyclopropyl-quinoxaline

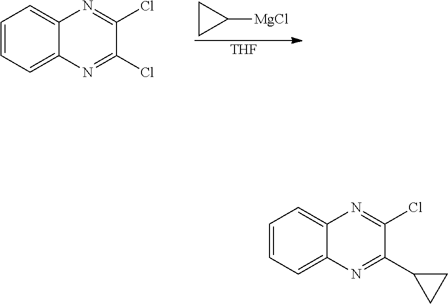

To a suspension of 2,3-dichloroquinoxaline (1.1 g, 5.53 mmol) in THF (8 mL) was added cyclopropylmagnesium bromide solution (1 M in THF, 20 mL) at 0° C. After being stirred at rt for 2 h, the mixture was poured to sat. NH₄Cl solution (60 mL). The mixture was extracted with EtOAc (80 mL), dried and concentrated. The resulting residue was purified by silica gel chromatography to provide the title compound (600 mg, yield: 53%) as light-yellow solid. MS (ESI) m/z=205.07 [M+H]⁺.

Step 2. Synthesis of 2-cyclopropyl-3-(1H-pyrazol-4-yl)quinoxaline

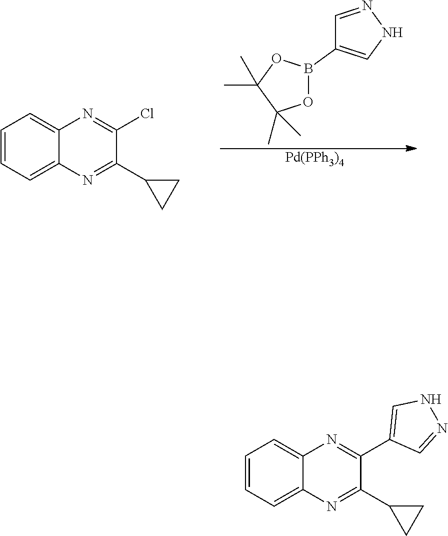 [SMILES: Clc1nc2ccccc2nc1C1CC1 ; CC1(C)OB(c2cn[nH]c2)OC1(C)C ; c1ccc2nc(C3CC3)c(-c3cn[nH]c3)nc2c1 ; "Pd(PPh3)4"]

A mixture of 2-chloro-3-cyclopropyl-quinoxaline (300 mg, 1.47 mmol), 4-(4,4,5,5-tetramethyl-1,3,2-dioxaborolan-2-yl)-1H-pyrazole (284.44 mg, 1.47 mmol), K₂CO₃ (203 mg, 1.47 mmol) and Pd(PPh₄)₃ (50 mg, 1.47 mmol) in toluene (10 mL), ethanol (5 mL) and water (2.5 mL) was heated at reflux overnight under N₂. The mixture was cooled to rt, and concentrated. The resulting residue was purified by reverse phase chromatography to provide the title compound (200 mg, yield: 57%) as light-yellow solid. MS (ESI) m/z=237.2 [M+H]⁺.

Step 3. Synthesis of tert-butyl (6-(4-(3-cyclopropylquinoxalin-2-yl)-1H-pyrazol-1-yl)hexyl)carbamate

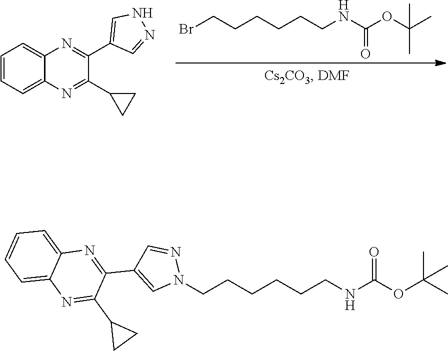 [SMILES: c1ccc2nc(C3CC3)c(-c3cn[nH]c3)nc2c1 ; CC(C)(C)OC(=O)NCCCCCCBr ; CC(C)(C)OC(=O)NCCCCCCn1cc(-c2nc3ccccc3nc2C2CC2)cn1]

A mixture of 2-cyclopropyl-3-(1H-pyrazol-4-yl)quinoxaline (100 mg, 419.01 μmol), tert-butyl N-(6-bromohexyl)carbamate (160 mg, 571.02 μmol), Cs₂CO₃ (270 mg, 828.68 μmol) and NaI (60 mg, 400.28 μmol) in DMF (2 mL) was heated at 90° C. overnight. The mixture was cooled to rt, and concentrated. The resulting residue was purified by reverse phase chromatography to provide the title compound (60 mg, yield: 33%) as white solid. MS (ESI) m/z=436.4 [M+H]⁺.

Step 4. Synthesis of 5-((6-(4-(3-cyclopropylquinoxalin-2-yl)-1H-pyrazol-1-yl)hexyl)amino)-2-(2,6-dioxopiperidin-3-yl)isoindoline-1,3-dione

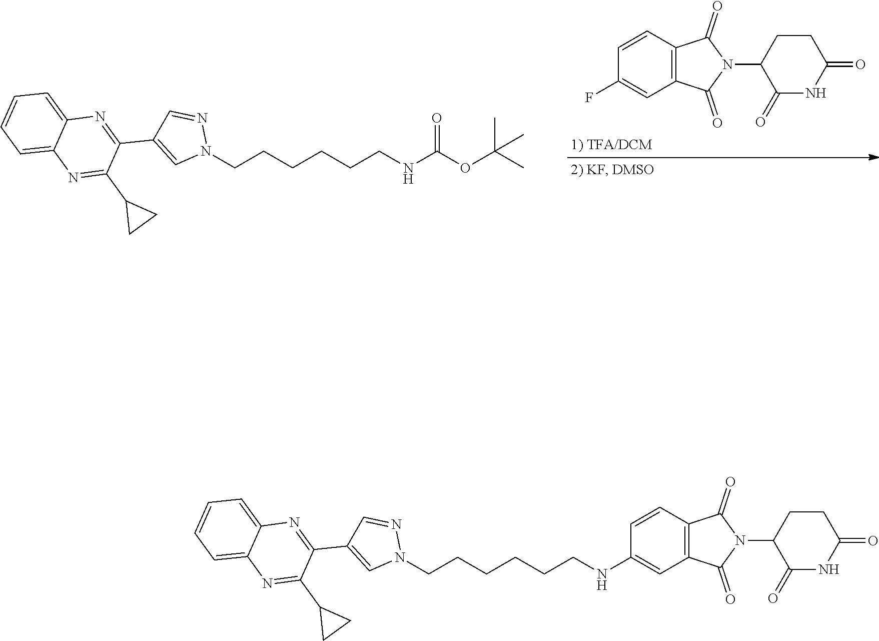 [SMILES: CC(C)(C)OC(=O)NCCCCCCn1cc(-c2nc3ccccc3nc2C2CC2)cn1 ; O=C1CCC(N2C(=O)c3ccc(F)cc3C2=O)C(=O)N1 ; O=C1CCC(N2C(=O)c3ccc(NCCCCCCn4cc(-c5nc6ccccc6nc5C5CC5)cn4)cc3C2=O)C(=O)N1]

To a solution of tert-butyl (6-(4-(3-cyclopropylquinoxalin-2-yl)-1H-pyrazol-1-yl)hexyl)carbamate (60 mg, 136.38 μmol) in DCM (3 mL) was added TFA (1 mL) at rt. After being stirred for 1 h, the mixture was concentrated. The resulting residue was dissolved in DCM (30 mL), and washed with sat. Na₂CO₃ solution (10 mL) and brine (10 mL). The organic phase was dried and concentrated. The resulting residue was dissolved in DMSO (2 mL). 2-(2,6-Dioxo-3-piperidyl)-5-fluoro-isoindoline-1,3-dione (80 mg, 289.62 μmol) and KF (20 mg, 344.25 μmol) were added. The obtained mixture was heated at 130° C. under microwave irradiation for 15 min. The obtained mixture was cooled to rt, and concentrated. The resulting residue was purified by reverse phase chromatography to provide the title compound (18 mg, yield: 22%) as yellow solid. MS (ESI) m/z=592.5 [M+H]⁺.

### Example 480. 2-(2,6-Dioxopiperidin-3-yl)-5-((6-(4-(3-(trifluoromethyl)quinoxalin-2-yl)-1H-pyrazol-1-yl)hexyl)amino)isoindoline-1,3-dione (GS-522)

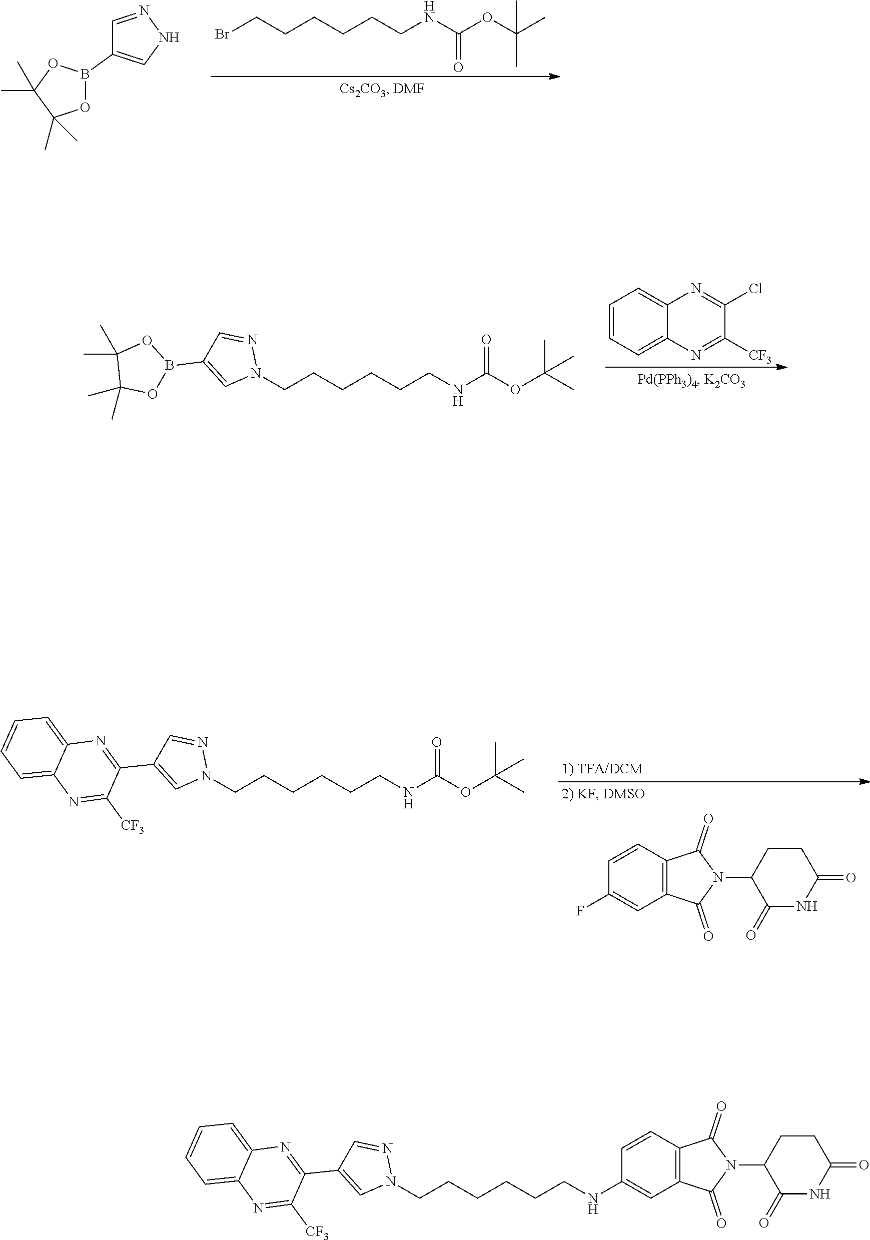

Step 1. Synthesis of tert-butyl (6-(4-(4,4,5,5-tetramethyl-1,3,2-dioxaborolan-2-yl)-1H-pyrazol-1-yl)hexyl)carbamate

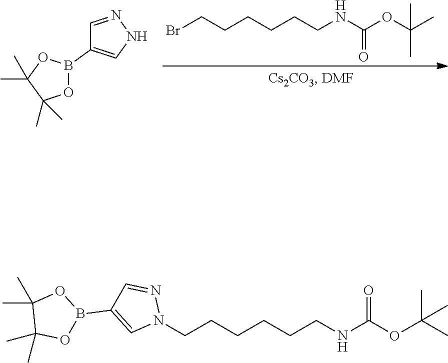 [SMILES: CC1(C)OB(c2cn[nH]c2)OC1(C)C ; CC(C)(C)OC(=O)NCCCCCCBr ; CC(C)(C)OC(=O)NCCCCCCn1cc(B2OC(C)(C)C(C)(C)O2)cn1]

A mixture of 4-(4,4,5,5-tetramethyl-1,3,2-dioxaborolan-2-yl)-1H-pyrazole (200 mg, 1.03 mmol), tert-butyl N-(6-bromohexyl)carbamate (300 mg, 1.07 mmol), Cs₂CO₃ (335.83 mg, 1.03 mmol) and NaI (154.50 mg, 1.03 mmol) in DMF (3 mL) was heated at 80° C. overnight. Then, the mixture was poured to water (30 mL), and extracted with EtOAc (30 mL×2). The combined organic layers were washed with water (30 mL), brine (30 mL), dried over Na₂SO₄, and concentrated. The resulting residue was purified by silica gel chromatography to provide the title compound (100 mg, yield: 22%) as light brown oil.

Step 2. Synthesis of tert-butyl (6-(4-(3-(trifluoromethyl)quinoxalin-2-yl)-1H-pyrazol-1-yl)hexyl)carbamate

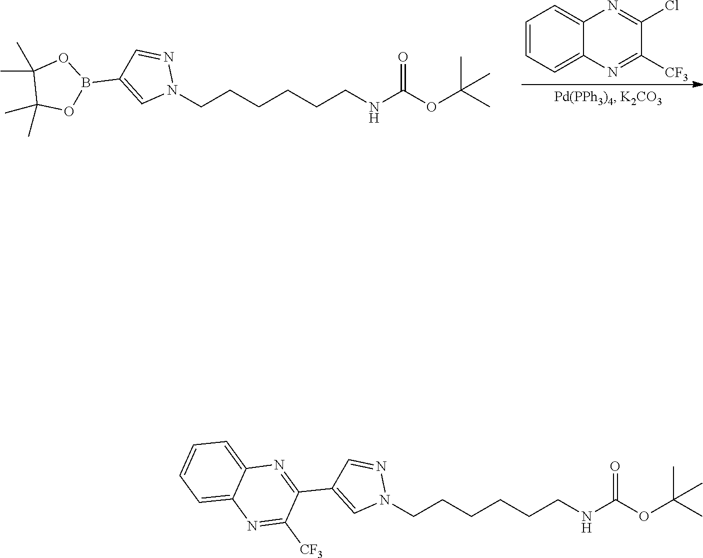

A mixture of tert-butyl (6-(4-(4,4,5,5-tetramethyl-1,3,2-dioxaborolan-2-yl)-1H-pyrazol-1-yl)hexyl)carbamate (100 mg, 228.82 μmol), 2-chloro-3-(trifluoromethyl)quinoxaline (80 mg, 343.95 μmol), K₂CO₃ (31.62 mg, 228.82 μmol) and Pd(PPh₃)₄ (30 mg, 228.82 μmol) in toluene (3 mL), ethanol (1.5 mL) and water (1.5 mL) was heated at reflux overnight under N₂. The mixture was cooled to rt and concentrated. The resulting residue was purified by reverse phase chromatography to provide the title compound (60 mg, yield: 35%) as white solid. MS (ESI) m/z=464.3 [M+H]⁺.

Step 3. Synthesis of 2-(2,6-dioxopiperidin-3-yl)-5-((6-(4-(3-(trifluoromethyl)quinoxalin-2-yl)-1H-pyrazol-1-yl)hexyl)amino)isoindoline-1,3-dione

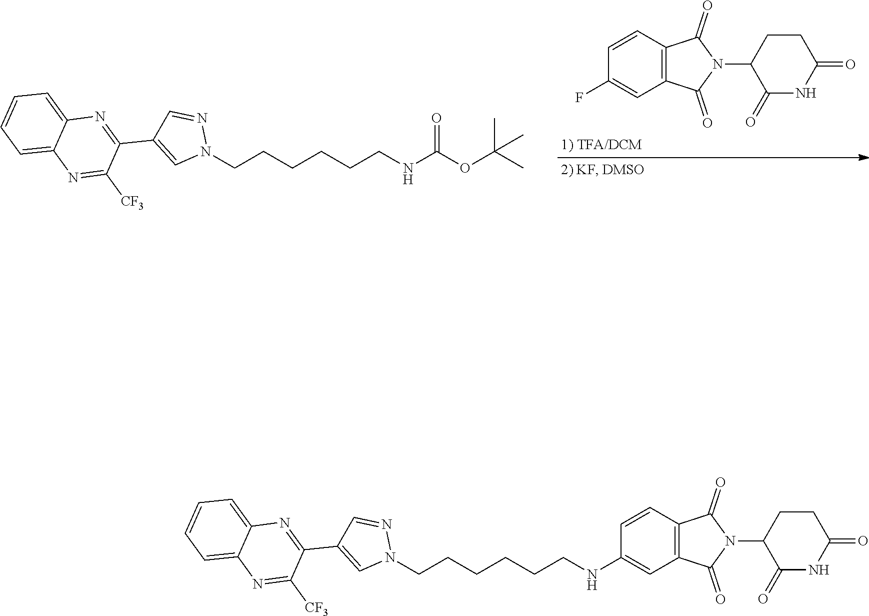 [SMILES: CC(C)(C)OC(=O)NCCCCCCn1cc(-c2nc3ccccc3nc2C(F)(F)F)cn1 ; O=C1CCC(N2C(=O)c3ccc(F)cc3C2=O)C(=O)N1 ; O=C1CCC(N2C(=O)c3ccc(NCCCCCCn4cc(-c5nc6ccccc6nc5C(F)(F)F)cn4)cc3C2=O)C(=O)N1]

To a solution of tert-butyl (6-(4-(3-(trifluoromethyl)quinoxalin-2-yl)-1H-pyrazol-1-yl)hexyl)carbamate (60 mg, 78.97 μmol) in DCM (3 mL) was added TFA (1 mL) at rt. After being stirred for 1 h, the mixture was concentrated. The resulting residue was dissolved in DCM (30 mL), washed with sat. Na₂CO₃ solution (10 mL), brine (10 mL), dried and concentrated. The resulting residue was dissolved in DMSO (2 mL). To the solution were added 2-(2,6-dioxo-3-piperidyl)-5-fluoro-isoindoline-1,3-dione (50 mg, 181.02 μmol) and KF (20 mg, 344.25 μmol). The mixture was heated at 130° C. under microwave irradiation for 15 min, and then cooled to rt. The obtained mixture was purified by reverse phase chromatography to provide the title compound (3 mg, yield: 6%) as yellow solid. MS (ESI) m/z=620.4 [M+H]⁺.

### Example 481. 2-(2,6-Dioxopiperidin-3-yl)-5-((6-(4-(3-isopropylquinoxalin-2-yl)-1H-pyrazol-1-yl)hexyl)amino)isoindoline-1,3-dione (GS-523)

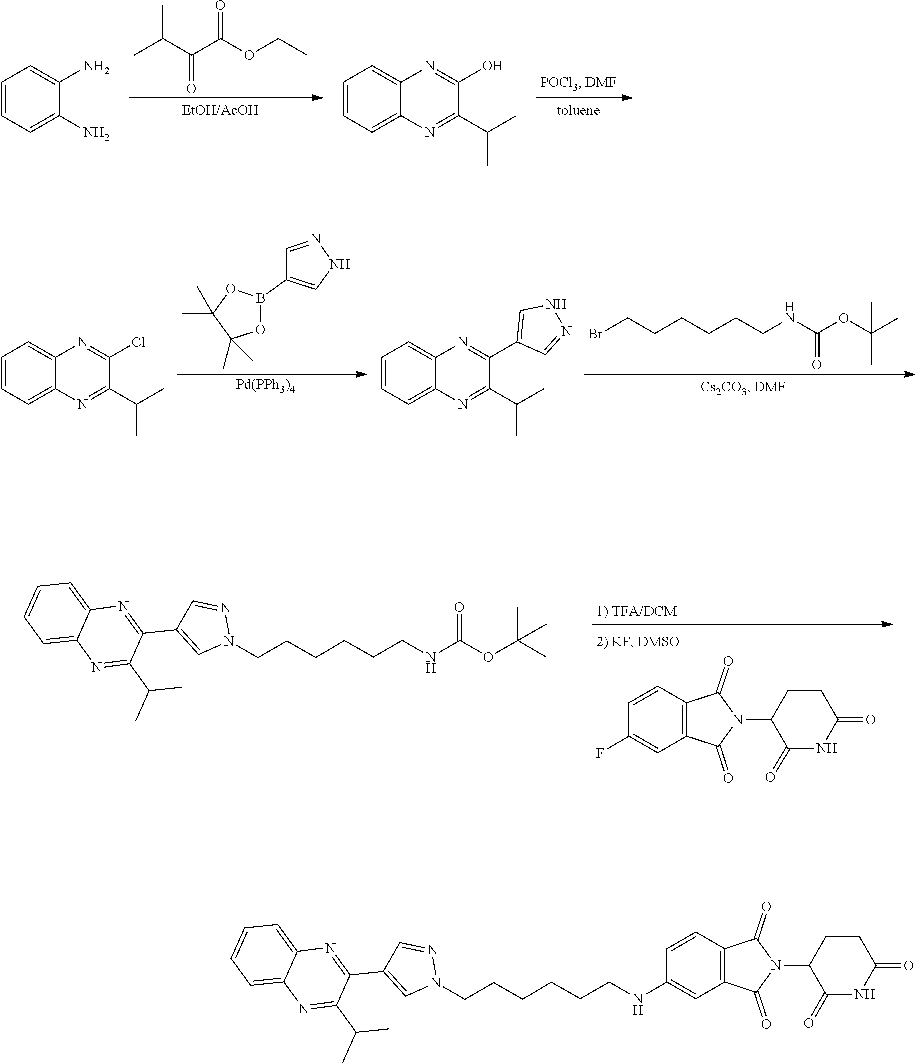

Step 1. Synthesis of 3-isopropylquinoxalin-2-ol

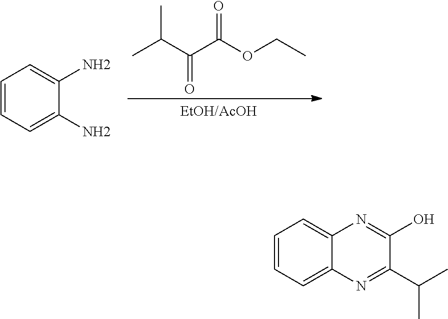

A mixture of benzene-1,2-diamine (1.50 g, 13.87 mmol) and ethyl 3-methyl-2-oxo-butanoate (2.0 g, 13.87 mmol) in ethanol (15 mL) and AcOH (1 mL) was heated at 80° C. overnight. The mixture was cooled to rt, and filtered to provide the title compound (2 g, yield: 77%) as white solid. MS (ESI) m/z=189.1 [M+H]⁺.

Step 2. Synthesis of 2-chloro-3-isopropyl-quinoxaline

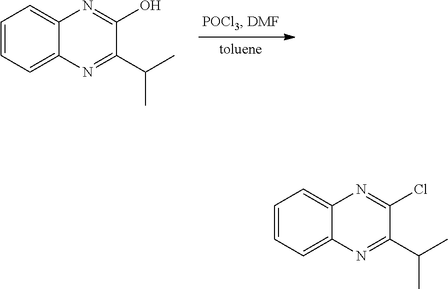

To a solution of 3-isopropylquinoxalin-2-ol (1.0 g, 5.31 mmol) in N,N-dimethylformamide (427.16 mg, 5.84 mmol) and toluene (10 mL) was added phosphoryl trichloride (1.06 g, 6.91 mmol) at rt. The reaction mixture was heated at 60° C. for 2 h. The mixture was poured into water (60 mL), and extracted with EtOAc (60 mL). The organic layer was washed with brine (30 mL), dried, and concentrated to provide the title compound (600 mg, yield: 24%) as light-yellow solid. MS (ESI) m/z=217.1 [M+H]⁺.

Step 3 to 5. Synthesis of 2-(2,6-Dioxopiperidin-3-yl)-5-((6-(4-(3-isopropylquinoxalin-2-yl)-1H-pyrazol-1-yl)hexyl)amino)isoindoline-1,3-dione

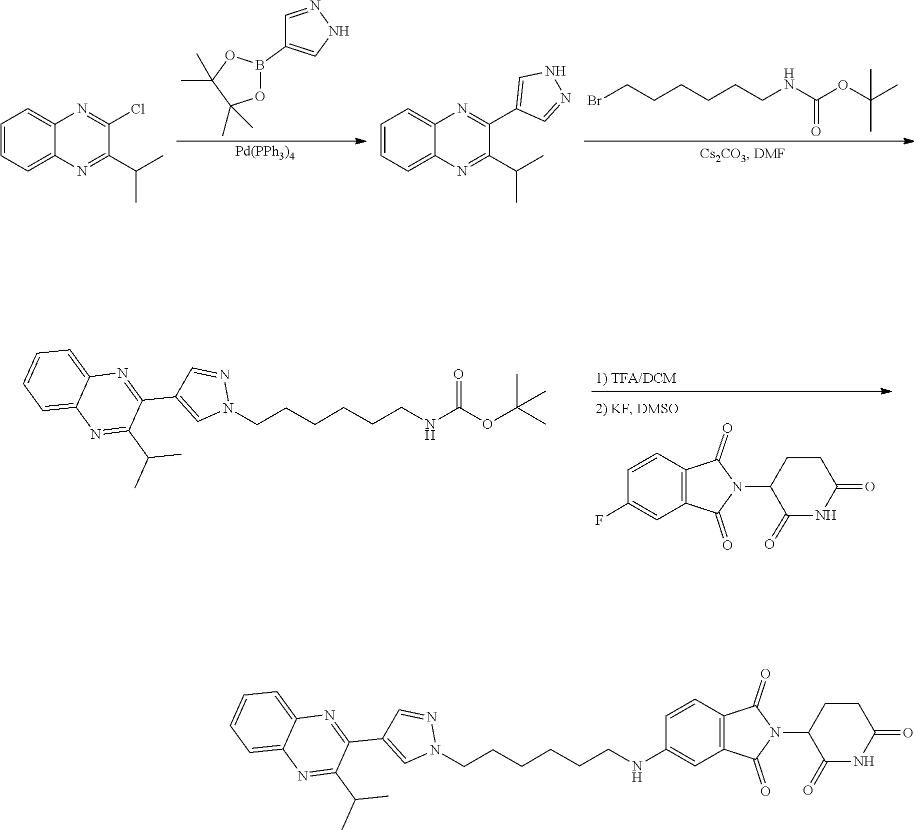

GS-523 was synthesized following the similar procedures as described for GS-521 (53 mg) as yellow solid. MS (ESI) m/z=594.6 [M+H]⁺.

### Example 482. 2-(2,6-Dioxopiperidin-3-yl)-5-((3-(4-(quinoxalin-2-yl)-1H-pyrazol-1-yl)cyclobutyl)amino)isoindoline-1,3-dione (GS-524)

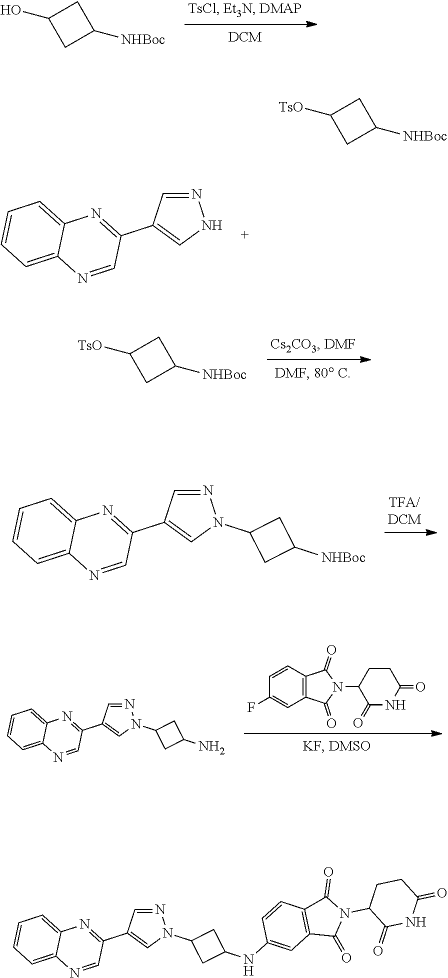

Step 1. Synthesis of 3-((tert-butoxycarbonyl)amino)cyclobutyl 4-methylbenzenesulfonate

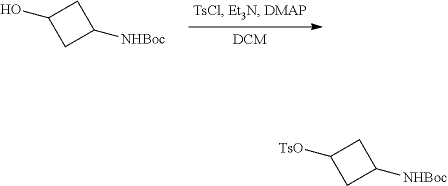 [SMILES: CC(C)(C)OC(=O)NC1CC(O)C1 ; Cc1ccc(S(=O)(=O)OC2CC(NC(=O)OC(C)(C)C)C2)cc1]

A solution of tert-butyl N-(3-hydroxycyclobutyl)carbamate (2 g, 10.68 mmol) in DCM (20 mL) was stirred at 0° C. To the solution was added 4-Methylbenzenesulfonyl chloride (2.04 g, 10.68 mmol) slowly. The mixture was stirred at rt overnight. Then, the mixture was concentrated. The resulting residue was purified by silica gel chromatography to provide the title compound (1 g, yield: 27%). MS (ESI) m/z=242.07 [M+H]⁺.

Step 2. Synthesis of tert-butyl (3-(4-(quinoxalin-2-yl)-1H-pyrazol-1-yl)cyclobutyl)carbamate

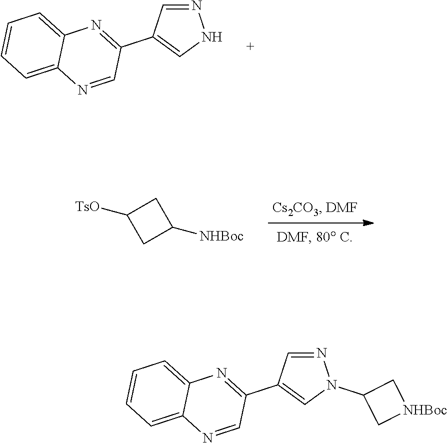

A solution of 2-(1H-pyrazol-4-yl)quinoxaline (50 mg, 254.83 μmol), 3-((tert-butoxycarbonyl)amino)cyclobutyl 4-methylbenzenesulfonate (113.11 mg, 331.28 μmol), K₂CO₃ (70.44 mg, 509.66 μmol), NaI (38.20 mg, 254.83 μmol) in DMF (1 mL) was stirred at 70° C. under N₂ for 2 h. Then, the mixture was concentrated. The resulting residue was purified by reverse phase chromatography to provide the title compound (90 mg, yield: 97%). MS (ESI) m/z=366.26 [M+H]⁺.

Step 3. Synthesis of 3-(4-(quinoxalin-2-yl)-1H-pyrazol-1-yl)cyclobutan-1-amine

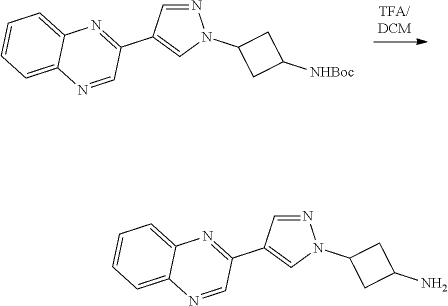 [SMILES: CC(C)(C)OC(=O)NC1CC(n2cc(-c3cnc4ccccc4n3)cn2)C1 ; NC1CC(n2cc(-c3cnc4ccccc4n3)cn2)C1]

A solution of tert-butyl (3-(4-(quinoxalin-2-yl)-1H-pyrazol-1-yl)cyclobutyl)carbamate (90 mg, 246.29 μmol) in DCM (6 mL) and TFA (2 mL) was stirred at rt for 1 h. Then, the reaction was concentrated. The resulting residue was purified by reverse phase chromatography to provide the title compound (60 mg, yield: 92%). MS (ESI) m/z=266.17 [M+H]⁺.

Step 4. Synthesis of 2-(2,6-dioxopiperidin-3-yl)-5-((3-(4-(quinoxalin-2-yl)-1H-pyrazol-1-yl)cyclobutyl)amino)isoindoline-1,3-dione

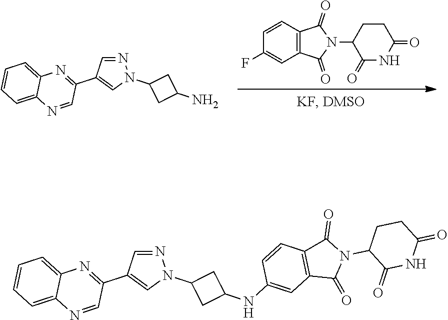 [SMILES: NC1CC(n2cc(-c3cnc4ccccc4n3)cn2)C1 ; O=C1CCC(N2C(=O)c3ccc(F)cc3C2=O)C(=O)N1 ; O=C1CCC(N2C(=O)c3ccc(NC4CC(n5cc(-c6cnc7ccccc7n6)cn5)C4)cc3C2=O)C(=O)N1]

A solution of 3-(4-(quinoxalin-2-yl)-1H-pyrazol-1-yl)cyclobutan-1-amine (30 mg, 113.07 μmol), 2-(2,6-dioxo-3-piperidyl)-5-fluoro-isoindoline-1,3-dione (46.85 mg, 169.61 μmol) and KF (13.14 mg, 226.15 μmol) in DMSO (1 mL) was irradiated at 130° C. in the microwave reactor for 15 min. The mixture was purified by reverse phase chromatography to provide the title compound (1.59 mg, yield: 3%). MS (ESI) m/z=522.3 [M+H]⁺.

### Example 483. 2-(2,6-Dioxopiperidin-3-yl)-5-((6-oxo-6-(4-(4-(7-(1,2,3,6-tetrahydropyridin-4-yl)quinoxalin-2-yl)-1H-pyrazol-1-yl)piperidin-1-yl)hexyl)amino)isoindoline-1,3-dione (GS-525)

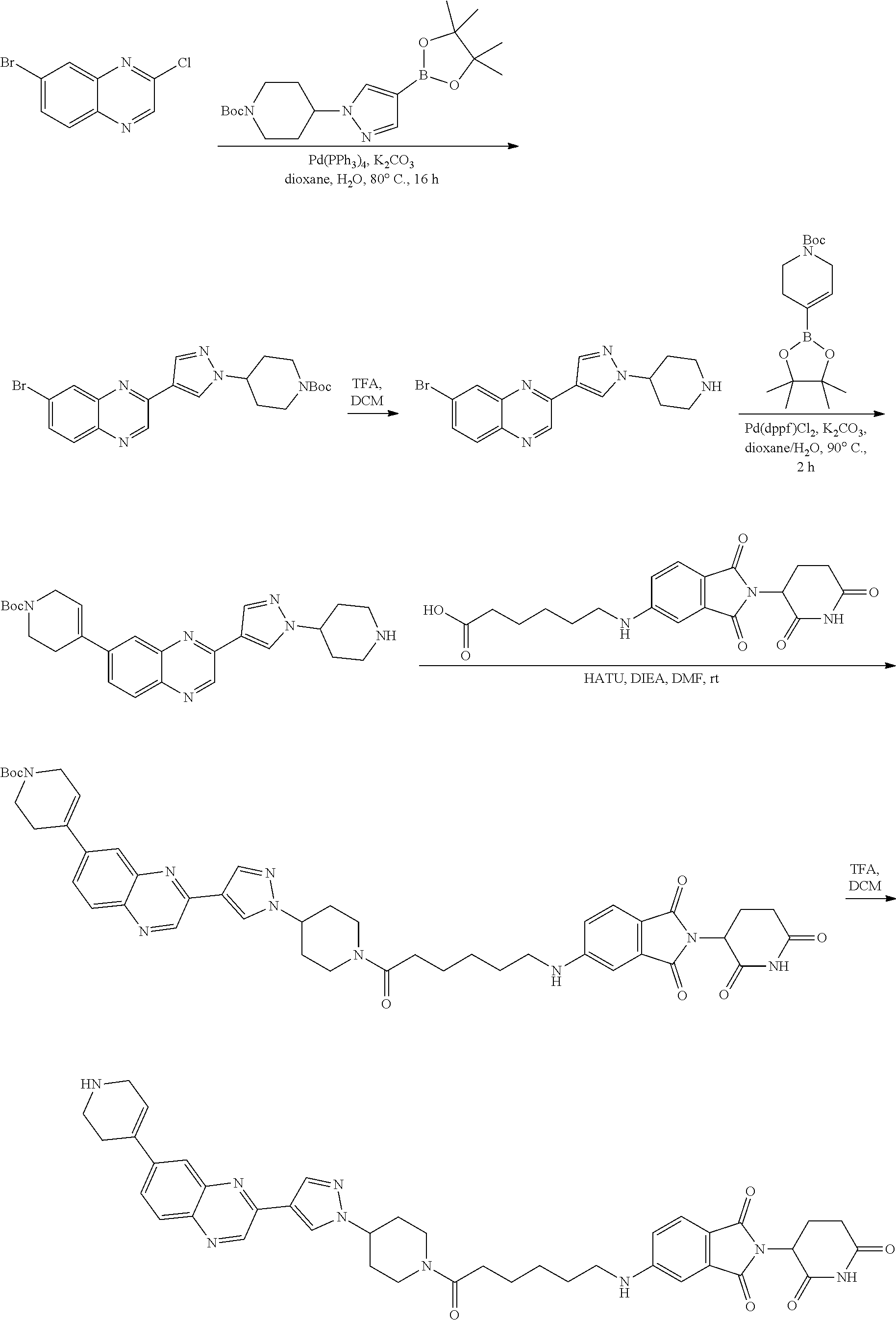

Step 1. Synthesis of tert-butyl 4-(4-(7-bromoquinoxalin-2-yl)-1H-pyrazol-1-yl)piperidine-1-carboxylate

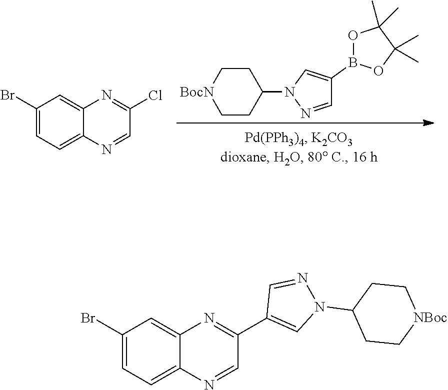 [SMILES: Clc1cnc2ccc(Br)cc2n1 ; CC(C)(C)OC(=O)N1CCC(n2cc(B3OC(C)(C)C(C)(C)O3)cn2)CC1 ; CC(C)(C)OC(=O)N1CCC(n2cc(-c3cnc4ccc(Br)cc4n3)cn2)CC1]

To a solution of 7-bromo-2-chloroquinoxaline (1 g, 4.1 mmol) in dioxane (20 mL) were added tert-butyl 4-(4-(4,4,5,5-tetramethyl-1,3,2-dioxaborolan-2-yl)-1H-pyrazol-1-yl)piperidine-1-carboxylate (1.56 g, 4.1 mmol), Pd(PPh₃)₄ (236 mg, 0.2 mmol), K₂CO₃ (1.13 g, 8.2 mmol) and H₂O (5 mL). The mixture was stirred at 80° C. for 16 h, and then concentrated. The resulting residue was purified by silica gel chromatography (petroleum ether:EtOAc=10:1) to provide the title compound (1.5 g, yield: 79%) as yellow solid. MS (ESI) m/z=403.3 [M−56+H]⁺.

Step 2. Synthesis of 7-bromo-2-(1-(piperidin-4-yl)-1H-pyrazol-4-yl)quinoxaline

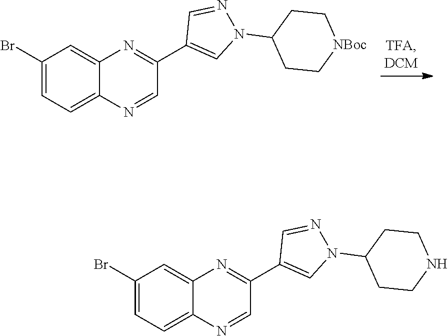 [SMILES: CC(C)(C)OC(=O)N1CCC(n2cc(-c3cnc4ccc(Br)cc4n3)cn2)CC1 ; Brc1ccc2ncc(-c3cnn(C4CCNCC4)c3)nc2c1]

To a solution of tert-butyl 4-(4-(7-bromoquinoxalin-2-yl)-1H-pyrazol-1-yl)piperidine-1-carboxylate (1.5 g, 3.28 mmol) in DCM (10 mL) was added TFA (3 mL). After the mixture was stirred at rt for 30 min, it was concentrated to provide the title compound (1.1 g, yield: 94%) as yellow solid, which was used directly without further purification. MS (ESI) m/z=358.4 [M+H]⁺.

Step 3. Synthesis of tert-butyl 4-(3-(1-(piperidin-4-yl)-1H-pyrazol-4-yl)quinoxalin-6-yl)-3,6-dihydropyridine-1(2H)-carboxylate

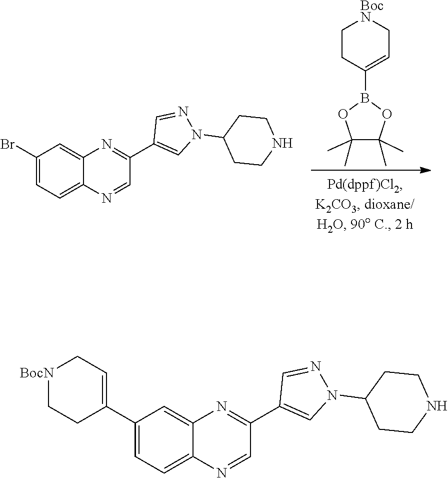

To a solution of 7-bromo-2-(1-(piperidin-4-yl)-1H-pyrazol-4-yl)quinoxaline (200 mg, 0.56 mmol) in dioxane (3 mL) were added tert-butyl 4-(4,4,5,5-tetramethyl-1,3,2-dioxaborolan-2-yl)-3,6-dihydropyridine-1(2H)-carboxylate (173 mg, 0.56 mmol), Pd(dppf)Cl₂ (20 mg, 0.028 mmol), K₂CO₃ (154 mg, 1.12 mmol) and H₂O (5 mL). The mixture was stirred at 90° C. for 2 h, and then concentrated. The resulting residue was purified by silica gel chromatography (petroleum ether:EtOAc=1:1) to provide the title compound (240 mg, yield: 93%) as yellow solid. MS (ESI) m/z=461.3 [M−56+H]⁺.

Step 4. Synthesis of tert-butyl 4-(3-(1-(1-(6-((2-(2,6-dioxopiperidin-3-yl)-1,3-dioxoisoindolin-5-yl)amino)hexanoyl)piperidin-4-yl)-1H-pyrazol-4-yl)quinoxalin-6-yl)-3,6-dihydropyridine-1(2H)-carboxylate

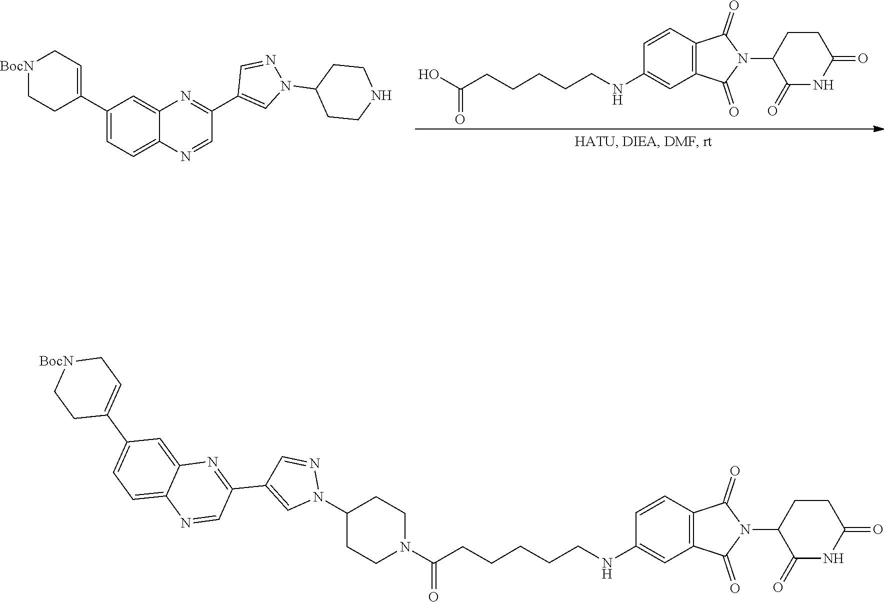 [SMILES: CC(C)(C)OC(=O)N1CC=C(c2ccc3ncc(-c4cnn(C5CCNCC5)c4)nc3c2)CC1 ; O=C(O)CCCCCNc1ccc2c(c1)C(=O)N(C1CCC(=O)NC1=O)C2=O ; CC(C)(C)OC(=O)N1CC=C(c2ccc3ncc(-c4cnn(C5CCN(C(=O)CCCCCNc6ccc7c(c6)C(=O)N(C6CCC(=O)NC6=O)C7=O)CC5)c4)nc3c2)CC1]

To a solution of tert-butyl 4-(3-(1-(piperidin-4-yl)-1H-pyrazol-4-yl)quinoxalin-6-yl)-3,6-dihydropyridine-1(2H)-carboxylate (20 mg, 0.04 mmol) in DMF (1 mL) were added 6-((2-(2,6-dioxopiperidin-3-yl)-1,3-dioxoisoindolin-5-yl)amino)hexanoic acid (15 mg, 0.04 mmol), HATU (22 mg, 0.06 mmol) and DIEA (15 mg, 0.12 mmol). After the mixture was stirred at rt for 2 h, it was purified by reverse phase chromatography (MeOH:H₂O=1:1) to provide the title compound (21 mg, yield: 58%) as yellow oil. MS (ESI) m/z=830.3 [M+H]⁺.

Step 5. Synthesis of 2-(2,6-dioxopiperidin-3-yl)-5-((6-oxo-6-(4-(4-(7-(1,2,3,6-tetrahydropyridin-4-yl)quinoxalin-2-yl)-1H-pyrazol-1-yl)piperidin-1-yl)hexyl)amino)isoindoline-1,3-dione

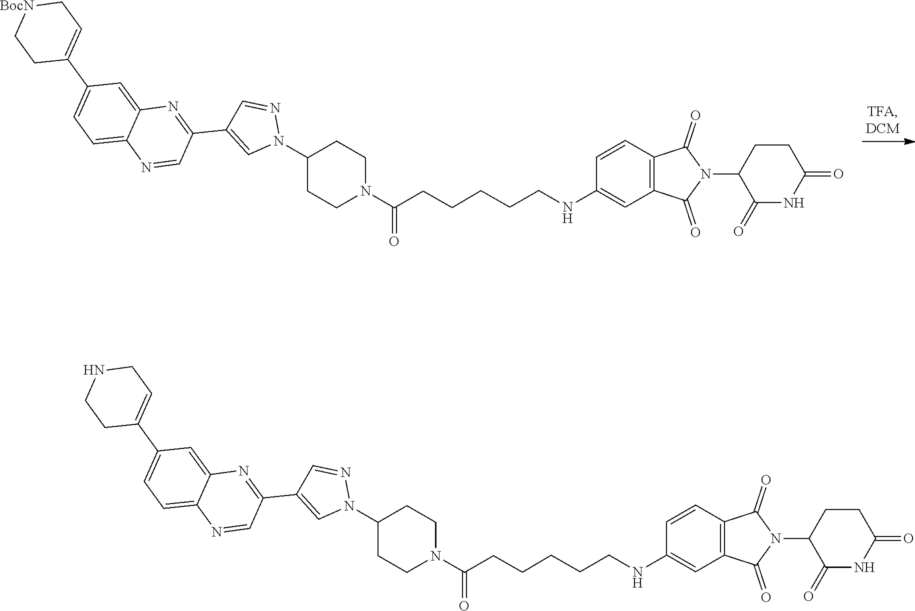 [SMILES: CC(C)(C)OC(=O)N1CC=C(c2ccc3ncc(-c4cnn(C5CCN(C(=O)CCCCCNc6ccc7c(c6)C(=O)N(C6CCC(=O)NC6=O)C7=O)CC5)c4)nc3c2)CC1 ; O=C1CCC(N2C(=O)c3ccc(NCCCCCC(=O)N4CCC(n5cc(-c6cnc7ccc(C8=CCNCC8)cc7n6)cn5)CC4)cc3C2=O)C(=O)N1]

To a solution of tert-butyl 4-(3-(1-(1-(6-((2-(2,6-dioxopiperidin-3-yl)-1,3-dioxoisoindolin-5-yl)amino)hexanoyl)piperidin-4-yl)-1H-pyrazol-4-yl)quinoxalin-6-yl)-3,6-dihydropyridine-1(2H)-carboxylate (21 mg, 0.02 mmol) in DCM (1 mL) was added TFA (1 mL). The mixture was stirred at rt for 30 min, and then purified by reverse phase chromatography (MeOH:H₂O=1:1) to provide the title compound (12 mg, yield: 65%) as yellow solid. MS (ESI) m/z=730.4 [M+H]⁺.

### Example 484. 2-(2,6-Dioxopiperidin-3-yl)-5-((6-oxo-6-(4-(4-(7-(piperazin-1-yl)quinoxalin-2-yl)-1H-pyrazol-1-yl)piperidin-1-yl)hexyl)amino)isoindoline-1,3-dione (GS-526)

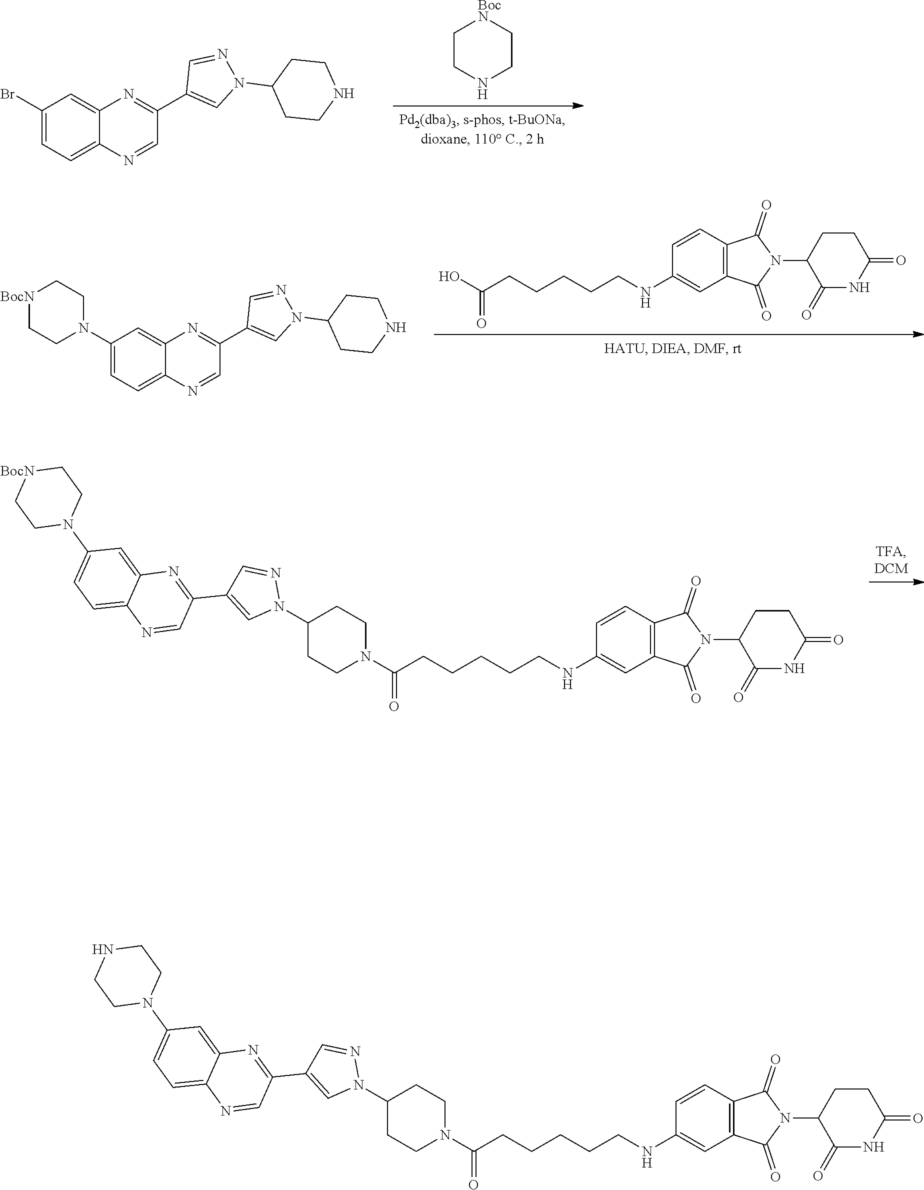 [SMILES: Brc1ccc2ncc(-c3cnn(C4CCNCC4)c3)nc2c1 ; CC(C)(C)OC(=O)N1CCNCC1 ; CC(C)(C)OC(=O)N1CCN(c2ccc3ncc(-c4cnn(C5CCNCC5)c4)nc3c2)CC1 ; O=C(O)CCCCCNc1ccc2c(c1)C(=O)N(C1CCC(=O)NC1=O)C2=O ; CC(C)(C)OC(=O)N1CCN(c2ccc3ncc(-c4cnn(C5CCN(C(=O)CCCCCNc6ccc7c(c6)C(=O)N(C6CCC(=O)NC6=O)C7=O)CC5)c4)nc3c2)CC1 ; O=C1CCC(N2C(=O)c3ccc(NCCCCCC(=O)N4CCC(n5cc(-c6cnc7ccc(N8CCNCC8)cc7n6)cn5)CC4)cc3C2=O)C(=O)N1]

GS-526 was synthesized following the similar procedures as described for GS-543 (5 mg) as red solid. MS (ESI) m/z=733.4 [M+H]⁺.

### Example 485. 2-(2,6-Dioxopiperidin-3-yl)-5-((6-(4-(quinoxalin-2-yl)-1H-pyrazol-1-yl)spiro[3.3]heptan-2-yl)amino)isoindoline-1,3-dione (GS-527)

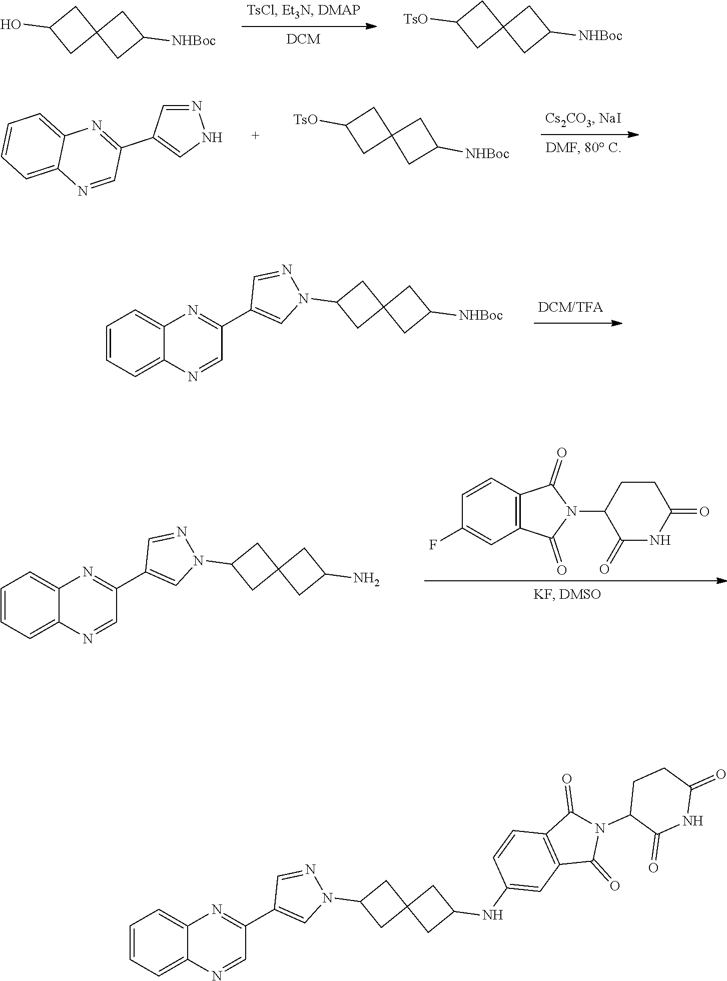

GS-527 was synthesized following the similar procedures as described for GS-524 (1.8 mg). MS (ESI) m/z=562.4 [M+H]⁺.

### Example 486. 2-(2,6-Dioxopiperidin-3-yl)-5-((6-(4-(4-(7-morpholinoquinoxalin-2-yl)-1H-pyrazol-1-yl)piperidin-1-yl)-6-oxohexyl)amino)isoindoline-1,3-dione (GS-528)

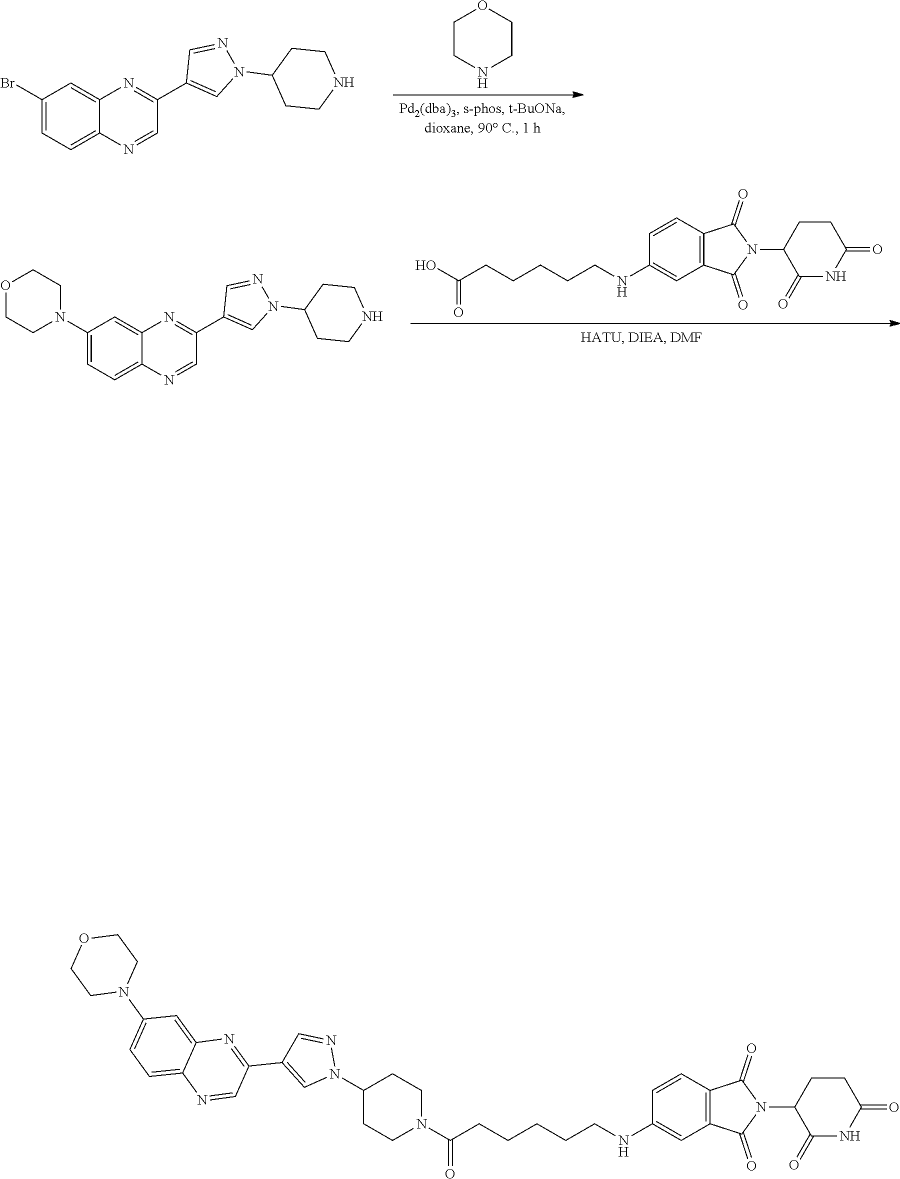 [SMILES: Brc1ccc2ncc(-c3cnn(C4CCNCC4)c3)nc2c1 ; C1COCCN1 ; c1nn(C2CCNCC2)cc1-c1cnc2ccc(N3CCOCC3)cc2n1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(n5cc(-c6cnc7ccc(N8CCOCC8)cc7n6)cn5)CC4)cc3C2=O)C(=O)N1]

GS-528 was synthesized following the similar procedures as described for GS-543 (4 mg) as red solid. MS (ESI) m/z=734.3 [M+H]⁺.

### Example 487. 2-(2,6-Dioxopiperidin-3-yl)-5-((6-(4-(7-(4-methylpiperazin-1-yl)quinoxalin-2-yl)-1H-pyrazol-1-yl)hexyl)amino)isoindoline-1,3-dione (GS-529)

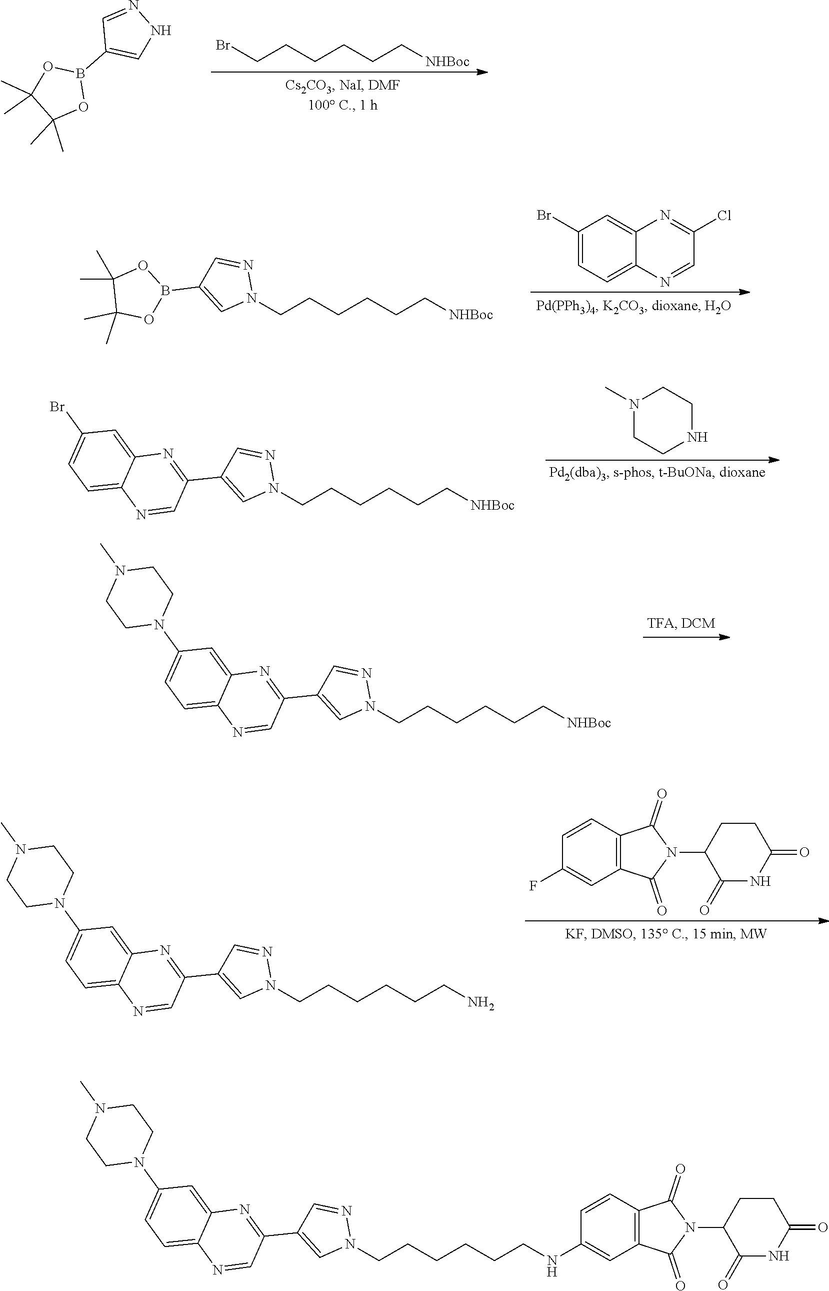

Step 1. Synthesis of tert-butyl (6-(4-(4,4,5,5-tetramethyl-1,3,2-dioxaborolan-2-yl)-1H-pyrazol-1-yl)hexyl)carbamate

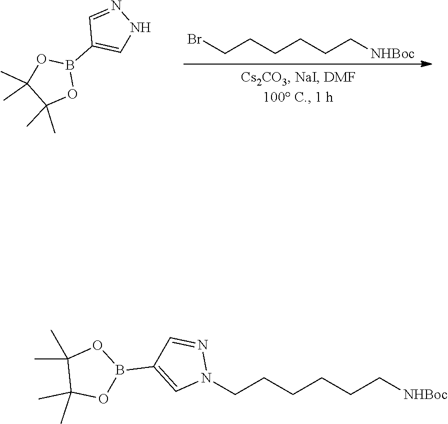 [SMILES: CC1(C)OB(c2cn[nH]c2)OC1(C)C ; CC(C)(C)OC(=O)NCCCCCCBr ; CC(C)(C)OC(=O)NCCCCCCn1cc(B2OC(C)(C)C(C)(C)O2)cn1]

To a solution of 4-(4,4,5,5-tetramethyl-1,3,2-dioxaborolan-2-yl)-1H-pyrazole (1 g, 5.1 mmol) in DMF (10 mL) were added tert-butyl (6-bromohexyl)carbamate (1.49 g, 5.3 mmol), Cs₂CO₃ (3.3 g, 102 mmol), NaI (765 mg, 5.1 mmol). After the mixture was stirred at 100° C. for 1 h, it was purified with reversed phase chromatography (MeOH:H₂O=2:1) to provide the title compound (1.7 g, yield: 84%) as yellow oil. MS (ESI) m/z=394.4 [M+H]⁺.

Step 2. Synthesis of tert-butyl (6-(4-(7-bromoquinoxalin-2-yl)-1H-pyrazol-1-yl)hexyl)carbamate

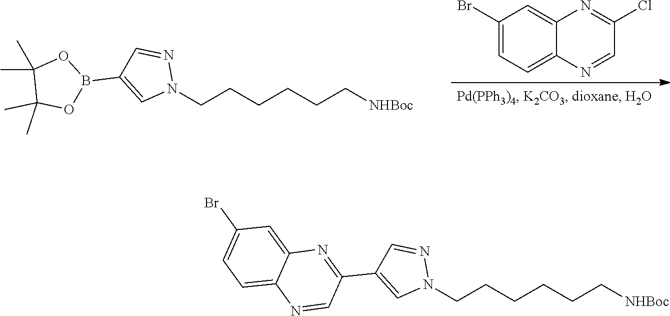 [SMILES: CC(C)(C)OC(=O)NCCCCCCn1cc(B2OC(C)(C)C(C)(C)O2)cn1 ; Clc1cnc2ccc(Br)cc2n1 ; CC(C)(C)OC(=O)NCCCCCCn1cc(-c2cnc3ccc(Br)cc3n2)cn1]

To a solution of tert-butyl (6-(4-(4,4,5,5-tetramethyl-1,3,2-dioxaborolan-2-yl)-1H-pyrazol-1-yl)hexyl)carbamate (800 mg, 2.03 mmol) in dioxane (10 mL) were added 7-bromo-2-chloroquinoxaline (492 mg, 2.03 mmol), Pd(PPh₃)₄ (115 mg, 0.1 mmol), K₂CO₃ (552 mg, 4.06 mmol) and H₂O (2 mL). The mixture was stirred at 90° C. for 2 h before it was concentrated. The resulting residue was purified with silica gel chromatography (petroleum ether:EtOAc=1:1) to provide the title compound (810 mg, yield: 84%) as yellow solid. MS (ESI) m/z=474.4 [M+H]⁺.

Step 3. Synthesis of tert-butyl (6-(4-(7-(4-methylpiperazin-1-yl)quinoxalin-2-yl)-1H-pyrazol-1-yl)hexyl)carbamate

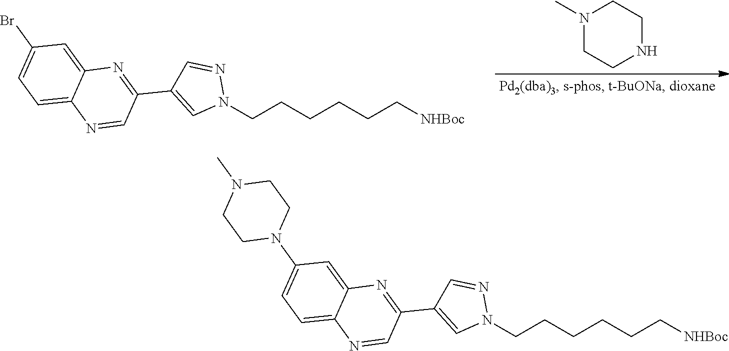

To a solution of tert-butyl (6-(4-(7-bromoquinoxalin-2-yl)-1H-pyrazol-1-yl)hexyl)carbamate (80 mg, 0.17 mmol) in 1,4-dioxane (2 mL) were added 1-methylpiperazine (33.8 mg, 0.34 mmol), Pd₂(dba)₃ (15 mg, 0.017 mmol), s-phos (14 mg, 0.34 mmol) and t-BuONa (32 mg, 0.34 mmol). The mixture was stirred at 100° C. for 1 h. The mixture was purified by reverse phase chromatography (MeOH:H₂O=1:1) to provide the title compound (71 mg, yield: 85%) as red oil. MS (ESI) m/z=494.4 [M+H]⁺.

Step 4. Synthesis of 6-(4-(7-(4-methylpiperazin-1-yl)quinoxalin-2-yl)-1H-pyrazol-1-yl)hexan-1-amine

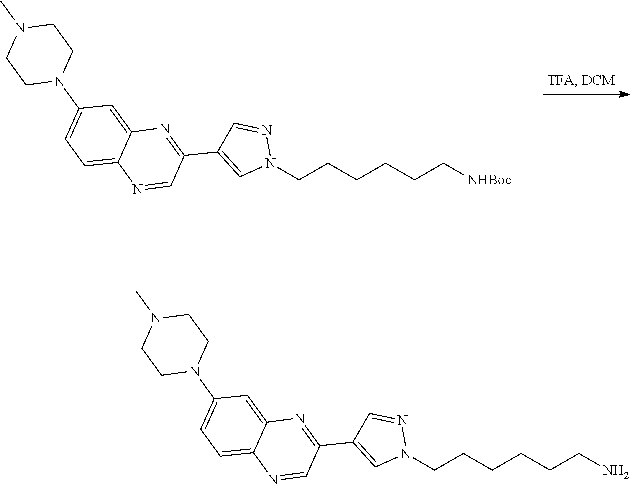 [SMILES: CN1CCN(c2ccc3ncc(-c4cnn(CCCCCCNC(=O)OC(C)(C)C)c4)nc3c2)CC1 ; CN1CCN(c2ccc3ncc(-c4cnn(CCCCCCN)c4)nc3c2)CC1]

To a solution of tert-butyl (6-(4-(7-(4-methylpiperazin-1-yl)quinoxalin-2-yl)-1H-pyrazol-1-yl)hexyl)carbamate (71 mg, 0.14 mmol) in DCM (1 mL) was added TFA (1 mL). The mixture was stirred at rt for 30 min, and then concentrated to provide the title compound (52 mg, yield: 92%) as red oil. MS (ESI) m/z=394.4 [M+H]⁺.

Step 5. Synthesis of 2-(2,6-dioxopiperidin-3-yl)-5-((6-(4-(7-(4-methylpiperazin-1-yl)quinoxalin-2-yl)-1H-pyrazol-1-yl)hexyl)amino)isoindoline-1,3-dione

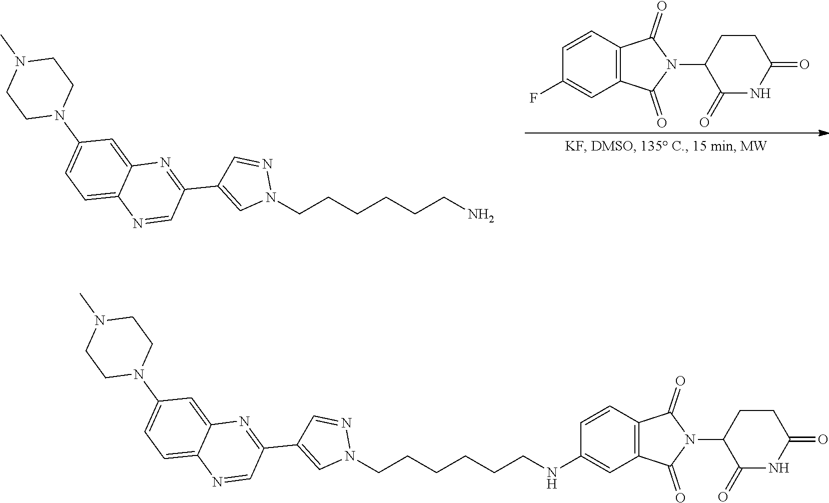 [SMILES: CN1CCN(c2ccc3ncc(-c4cnn(CCCCCCN)c4)nc3c2)CC1 ; O=C1CCC(N2C(=O)c3ccc(F)cc3C2=O)C(=O)N1 ; CN1CCN(c2ccc3ncc(-c4cnn(CCCCCCNc5ccc6c(c5)C(=O)N(C5CCC(=O)NC5=O)C6=O)c4)nc3c2)CC1]

To a solution of 6-(4-(7-(4-methylpiperazin-1-yl)quinoxalin-2-yl)-1H-pyrazol-1-yl)hexan-1-amine (20 mg, 0.05 mmol) in DMSO (1 mL) were added 2-(2,6-dioxopiperidin-3-yl)-5-fluoroisoindoline-1,3-dione (27.6 mg, 0.1 mmol), and KF (9 mg, 0.15 mmol). The mixture was stirred at 135° C. for 15 min, and then purified by reverse phase chromatography (MeOH:H₂O=1:1) to provide the title compound (5 mg, yield: 15%) as red solid. MS (ESI) m/z=650.5 [M+H]⁺.

### Example 488. 2-(2,6-Dioxopiperidin-3-yl)-5-((6-(4-(7-morpholinoquinoxalin-2-yl)-1H-pyrazol-1-yl)hexyl)amino)isoindoline-1,3-dione (GS-530)

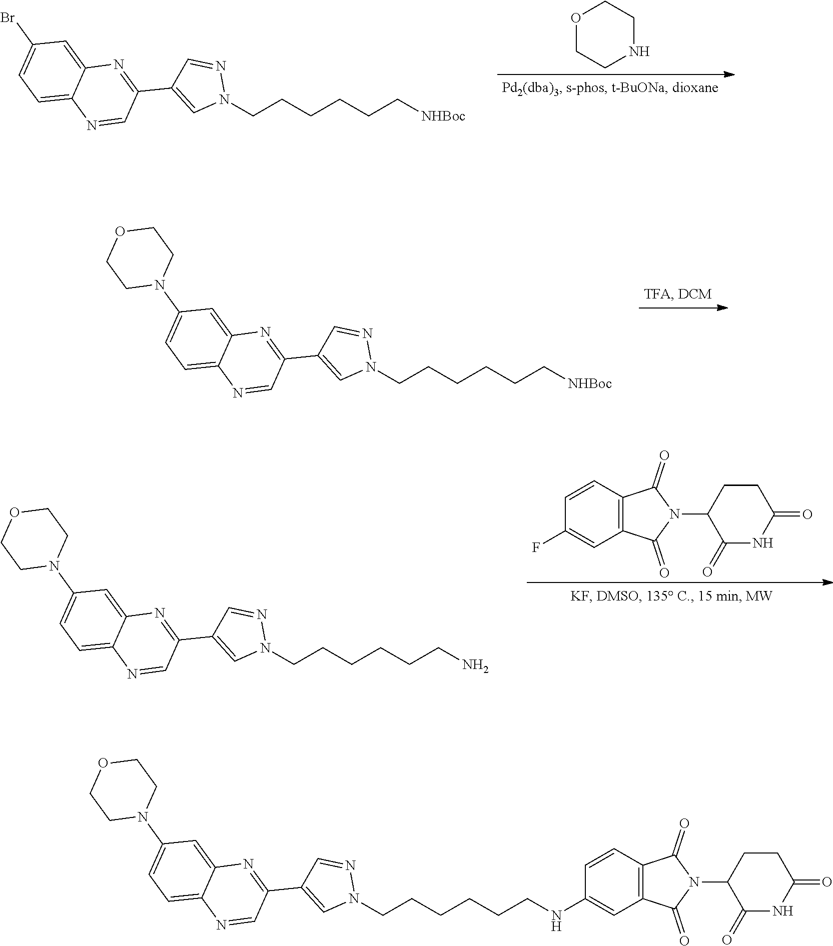 [SMILES: CC(C)(C)OC(=O)NCCCCCCn1cc(-c2cnc3ccc(Br)cc3n2)cn1 ; C1COCCN1 ; CC(C)(C)OC(=O)NCCCCCCn1cc(-c2cnc3ccc(N4CCOCC4)cc3n2)cn1 ; NCCCCCCn1cc(-c2cnc3ccc(N4CCOCC4)cc3n2)cn1 ; O=C1CCC(N2C(=O)c3ccc(F)cc3C2=O)C(=O)N1 ; O=C1CCC(N2C(=O)c3ccc(NCCCCCCn4cc(-c5cnc6ccc(N7CCOCC7)cc6n5)cn4)cc3C2=O)C(=O)N1]

GS-530 was synthesized following the similar procedures as described or GS-529 (4 mg as red solid. MS (ESI) m/z=637.3 [M+H]⁺.

### Example 489. 2-(2,6-Dioxopiperidin-3-yl)-5-((6-(4-(7-(1,2,3,6-tetrahydropyridin-4-yl)quinoxalin-2-yl)-1H-pyrazol-1-yl)hexyl)amino)isoindoline-1,3-dione (GS-531)

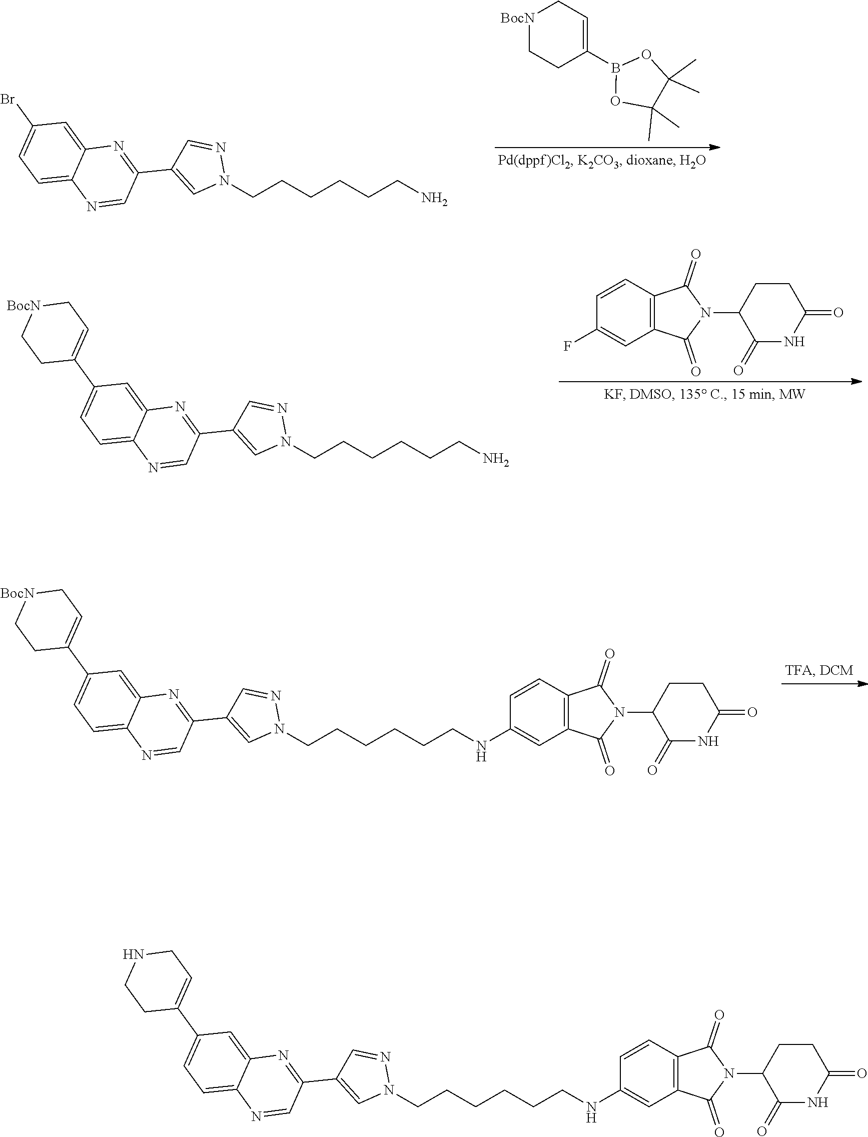 [SMILES: NCCCCCCn1cc(-c2cnc3ccc(Br)cc3n2)cn1 ; CC(C)(C)OC(=O)N1CC=C(B2OC(C)(C)C(C)(C)O2)CC1 ; CC(C)(C)OC(=O)N1CC=C(c2ccc3ncc(-c4cnn(CCCCCCN)c4)nc3c2)CC1 ; O=C1CCC(N2C(=O)c3ccc(F)cc3C2=O)C(=O)N1 ; CC(C)(C)OC(=O)N1CC=C(c2ccc3ncc(-c4cnn(CCCCCCNc5ccc6c(c5)C(=O)N(C5CCC(=O)NC5=O)C6=O)c4)nc3c2)CC1 ; O=C1CCC(N2C(=O)c3ccc(NCCCCCCn4cc(-c5cnc6ccc(C7=CCNCC7)cc6n5)cn4)cc3C2=O)C(=O)N1]

Step 1. Synthesis of tert-butyl 4-(3-(1-(6-aminohexyl)-1H-pyrazol-4-yl)quinoxalin-6-yl)-3,6-dihydropyridine-1(2H)-carboxylate

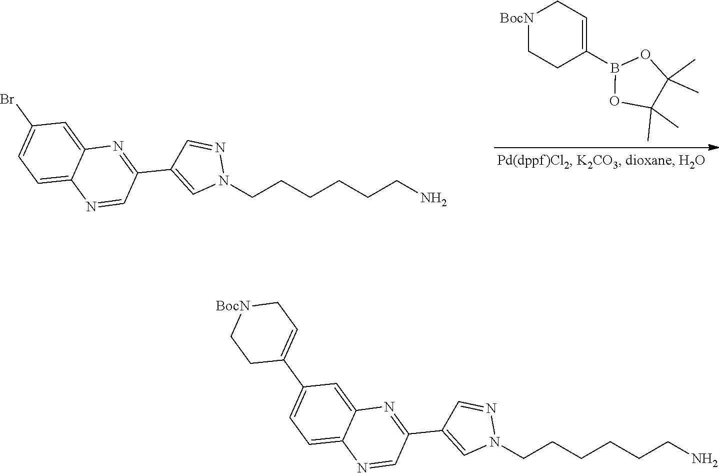 [SMILES: NCCCCCCn1cc(-c2cnc3ccc(Br)cc3n2)cn1 ; CC(C)(C)OC(=O)N1CC=C(B2OC(C)(C)C(C)(C)O2)CC1 ; CC(C)(C)OC(=O)N1CC=C(c2ccc3ncc(-c4cnn(CCCCCCN)c4)nc3c2)CC1]

To a solution of 6-(4-(7-bromoquinoxalin-2-yl)-1H-pyrazol-1-yl)hexan-1-amine (see step 1 of GS-532 for synthesis, 119 mg, 0.32 mmol) in 1,4-dioxane (3 mL) were added tert-butyl 4-(4,4,5,5-tetramethyl-1,3,2-dioxaborolan-2-yl)-3,6-dihydropyridine-1(2H)-carboxylate (148 mg, 0.48 mmol), Pd(dppf)Cl₂ (22 mg, 0.032 mmol), K₂CO₃ (88 mg, 0.64 mmol) and H₂O (0.5 mL). The mixture was stirred at 90° C. for 1 h. The mixture was purified by reverse phase chromatography (CH₃OH:H₂O=1:1) to provide the title compound (134 mg, yield: 89%) as red oil. MS (ESI) m/z=477.3 [M+H]⁺.

Step 2. Synthesis of tert-butyl 4-(3-(1-(6-((2-(2,6-dioxopiperidin-3-yl)-1,3-dioxoisoindolin-5-yl)amino)hexyl)-1H-pyrazol-4-yl)quinoxalin-6-yl)-3,6-dihydropyridine-1(2H)-carboxylate

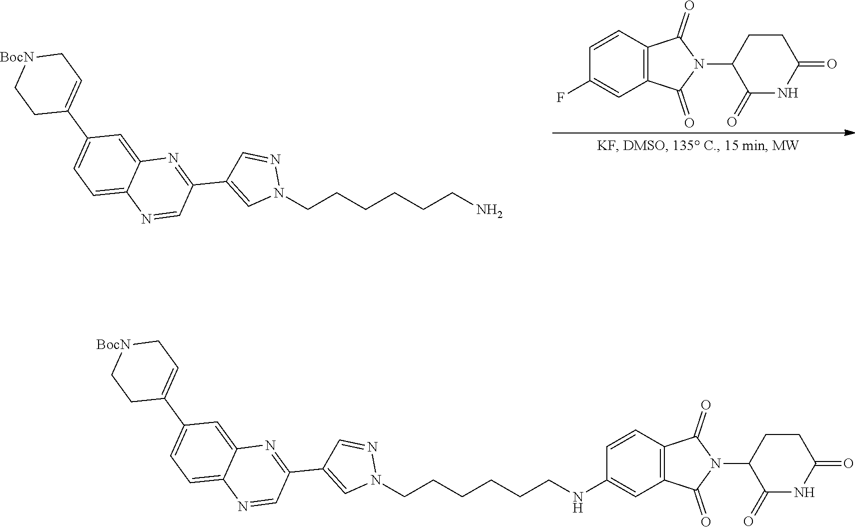 [SMILES: CC(C)(C)OC(=O)N1CC=C(c2ccc3ncc(-c4cnn(CCCCCCN)c4)nc3c2)CC1 ; O=C1CCC(N2C(=O)c3ccc(F)cc3C2=O)C(=O)N1 ; CC(C)(C)OC(=O)N1CC=C(c2ccc3ncc(-c4cnn(CCCCCCNc5ccc6c(c5)C(=O)N(C5CCC(=O)NC5=O)C6=O)c4)nc3c2)CC1]

To a solution of tert-butyl 4-(3-(1-(6-aminohexyl)-1H-pyrazol-4-yl)quinoxalin-6-yl)-3,6-dihydropyridine-1(2H)-carboxylate (20 mg, 0.04 mmol) in DMSO (1 mL) was added 2-(2,6-dioxopiperidin-3-yl)-5-fluoroisoindoline-1,3-dione (22.1 mg, 0.08 mmol) and KF (7.2 mg, 0.12 mmol). The mixture was stirred at 135° C. for 15 min. The resulting residue was purified by reverse phase chromatography (MeOH:H₂O=1:1) to provide the title compound (7 mg, yield: 23%) as red oil. MS (ESI) m/z=733.4 [M+H]⁺.

Step 3. Synthesis of 2-(2,6-dioxopiperidin-3-yl)-5-((6-(4-(7-(1,2,3,6-tetrahydropyridin-4-yl)quinoxalin-2-yl)-1H-pyrazol-1-yl)hexyl)amino)isoindoline-1,3-dione

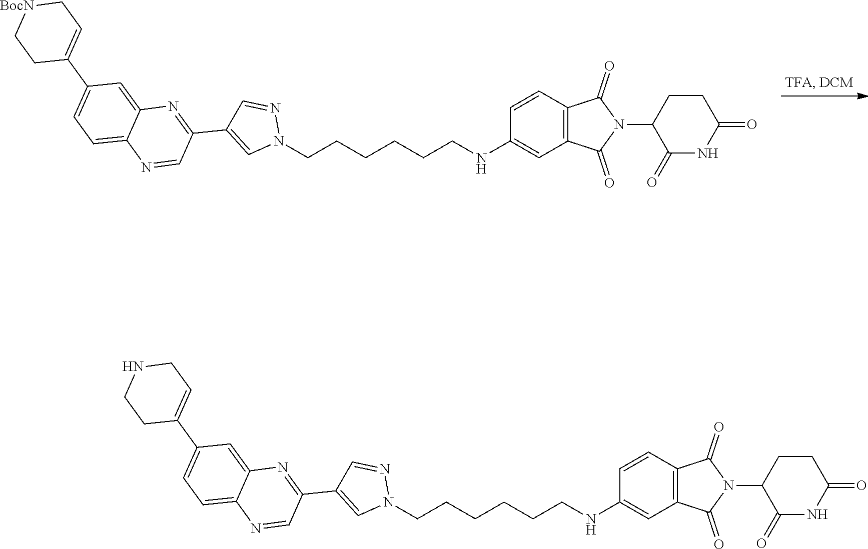 [SMILES: CC(C)(C)OC(=O)N1CC=C(c2ccc3ncc(-c4cnn(CCCCCCNc5ccc6c(c5)C(=O)N(C5CCC(=O)NC5=O)C6=O)c4)nc3c2)CC1 ; O=C1CCC(N2C(=O)c3ccc(NCCCCCCn4cc(-c5cnc6ccc(C7=CCNCC7)cc6n5)cn4)cc3C2=O)C(=O)N1]

To a solution of tert-butyl 4-(3-(1-(6-((2-(2,6-dioxopiperidin-3-yl)-1,3-dioxoisoindolin-5-yl)amino)hexyl)-1H-pyrazol-4-yl)quinoxalin-6-yl)-3,6-dihydropyridine-1(2H)-carboxylate (7 mg, 0.009 mmol) in DCM (1 mL) was added TFA (1 mL). The mixture was stirred at it for 30 min, and purified by reverse phase chromatography (MeOH:H₂O=1:1) to provide the title compound (4 mg, yield: 66%) as red solid. MS (ESI) m/z=633.4 [M+H]⁺.

### Example 490. 2-(2,6-Dioxopiperidin-3-yl)-5-((6-(4-(7-(piperazin-1-yl)quinoxalin-2-yl)-1H-pyrazol-1-yl)hexyl)amino)isoindoline-1,3-dione (GS-532)

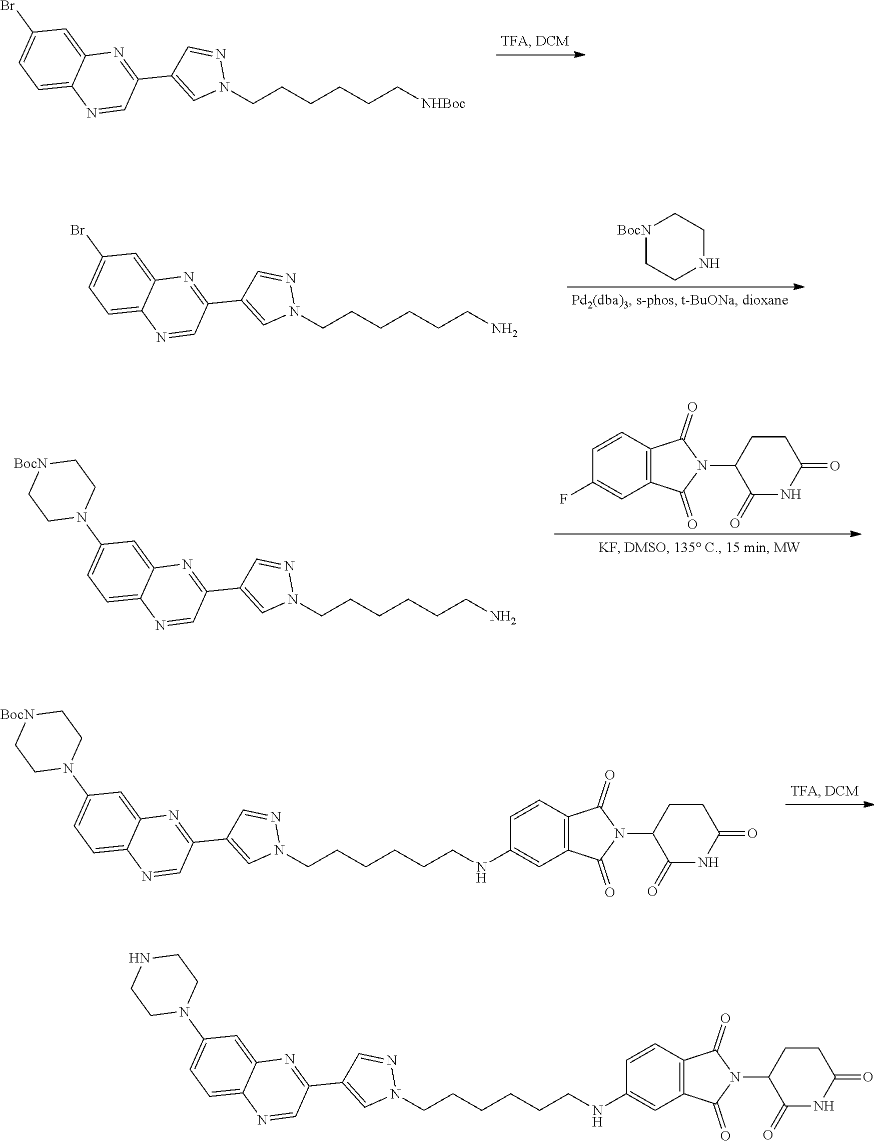

Step 1. Synthesis of 6-(4-(7-bromoquinoxalin-2-yl)-1H-pyrazol-1-yl)hexan-1-amine

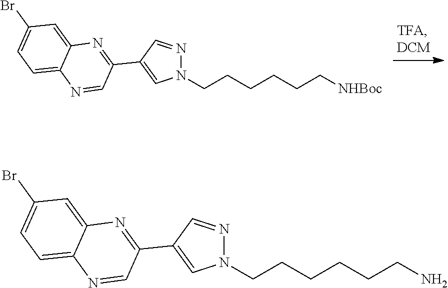 [SMILES: CC(C)(C)OC(=O)NCCCCCCn1cc(-c2cnc3ccc(Br)cc3n2)cn1 ; NCCCCCCn1cc(-c2cnc3ccc(Br)cc3n2)cn1]

To a solution of tert-butyl (6-(4-(7-bromoquinoxalin-2-yl)-1H-pyrazol-1-yl)hexyl)carbamate (see step 2 of GS-529 for synthesis, 500 mg, 1.05 mmol) in DCM (2 mL) was added TFA (2 mL). The mixture was stirred at it for 30 min, and then concentrated. The resulting residue was used directly without further purification. MS (ESI) m/z=374.3 [M+H]⁺.

Synthesis of tert-butyl 4-(3-(1-(6-aminohexyl)-1H-pyrazol-4-yl)quinoxalin-6-yl)piperazine-1-carboxylate

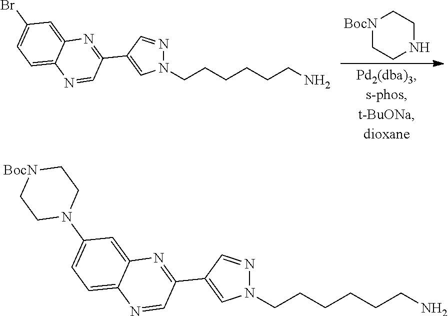 [SMILES: NCCCCCCn1cc(-c2cnc3ccc(Br)cc3n2)cn1 ; CC(C)(C)OC(=O)N1CCNCC1 ; CC(C)(C)OC(=O)N1CCN(c2ccc3ncc(-c4cnn(CCCCCCN)c4)nc3c2)CC1]

To a solution of 6-(4-(7-bromoquinoxalin-2-yl)-1H-pyrazol-1-yl)hexan-1-amine (50 mg, 0.13 mmol) in 1,4-dioxane (1 mL) was added tert-butyl piperazine-1-carboxylate (49 mg, 0.26 mmol), Pd₂(dba)₃ (12 mg, 0.013 mmol), s-phos (10 mg, 0.026 mmol) and t-BuONa (25 mg, 0.26 mmol). The mixture was stirred at 90° C. for 1 h, and then purified by reverse phase chromatography (CH₃OH:H₂O=1:1) to provide the title compound (52 mg, yield: 81%) as red oil. MS (ESI) m/z=480.3 [M+H]⁺.

Step 3. Synthesis of tert-butyl 4-(3-(1-(6-((2-(2,6-dioxopiperidin-3-yl)-1,3-dioxoisoindolin-5-yl)amino)hexyl)-1H-pyrazol-4-yl)quinoxalin-6-yl)piperazine-1-carboxylate

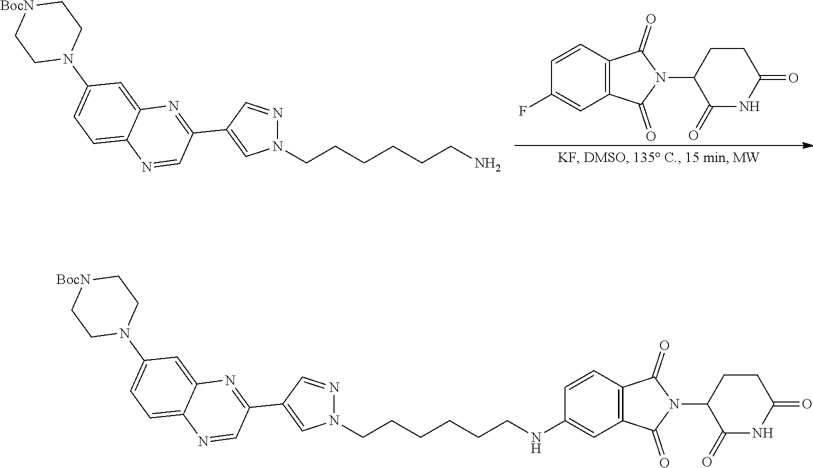 [SMILES: CC(C)(C)OC(=O)N1CCN(c2ccc3ncc(-c4cnn(CCCCCCN)c4)nc3c2)CC1 ; O=C1CCC(N2C(=O)c3ccc(F)cc3C2=O)C(=O)N1 ; CC(C)(C)OC(=O)N1CCN(c2ccc3ncc(-c4cnn(CCCCCCNc5ccc6c(c5)C(=O)N(C5CCC(=O)NC5=O)C6=O)c4)nc3c2)CC1]

To a solution of tert-butyl-4-(3-(1-aminohexyl)-1H-pyrazol-4-yl)quinoxalin-6-yl) piperazine-1-carboxylate (20 mg, 0.04 mmol) in DMSO (1 mL) was added 2-(2,6-dioxopiperidin-3-yl)-5-fluoroisoindoline-1,3-dione (22.1 mg, 0.08 mmol) and KF (7.2 mg, 0.12 mmol). The mixture was stirred at 135° C. for 15 min, and then purified by reverse phase chromatography (MeOH:H₂O=1:1) to provide the title compound (8 mg, yield: 26%) as red oil. MS (ESI) m/z=736.4 [M+H]⁺.

Step 4. Synthesis of 2-(2,6-dioxopiperidin-3-yl)-5-((6-(4-(7-(piperazin-1-yl)quinoxalin-2-yl)-1H-pyrazol-1-yl)hexyl)amino)isoindoline-1,3-dione

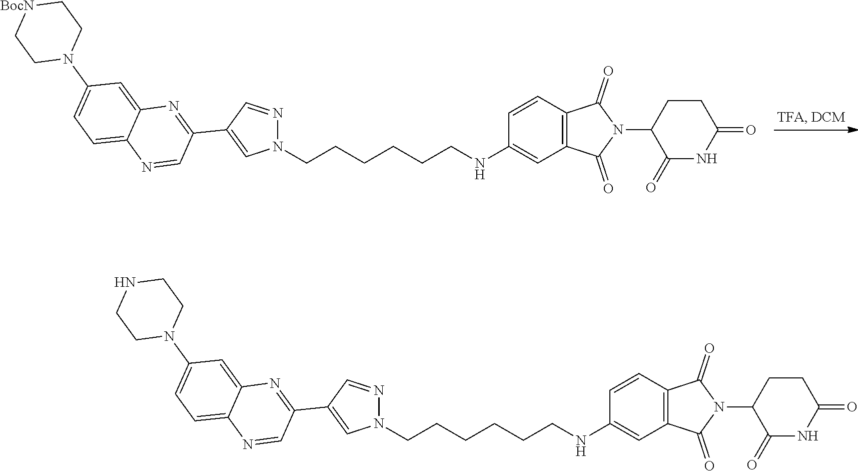 [SMILES: CC(C)(C)OC(=O)N1CCN(c2ccc3ncc(-c4cnn(CCCCCCNc5ccc6c(c5)C(=O)N(C5CCC(=O)NC5=O)C6=O)c4)nc3c2)CC1 ; O=C1CCC(N2C(=O)c3ccc(NCCCCCCn4cc(-c5cnc6ccc(N7CCNCC7)cc6n5)cn4)cc3C2=O)C(=O)N1]

To a solution of tert-butyl 4-(3-(1-(6-((2-(2,6-dioxopiperidin-3-yl)-1,3-dioxoisoindolin-5-yl)amino)hexyl)-1H-pyrazol-4-yl)quinoxalin-6-yl)piperazine-1-carboxylate (8 mg, 0.01 mmol) in DCM (1 mL) was added TFA (1 mL). The mixture was stirred at rt for 30 min, and purified by reverse phase chromatography (MeOH:H₂O=1:1) to provide the title compound (4 mg, yield: 58%) as red solid. MS (ESI) m/z=636.3 [M+H]⁺.

### Example 491. N-((2-(2,6-dioxopiperidin-3-yl)-1-oxoisoindolin-5-yl)methyl)-2,2-difluoro-5-oxo-5-(4-(4-(quinoxalin-2-yl)-1H-pyrazol-1-yl)piperidin-1-yl)pentanamide (GS-533)

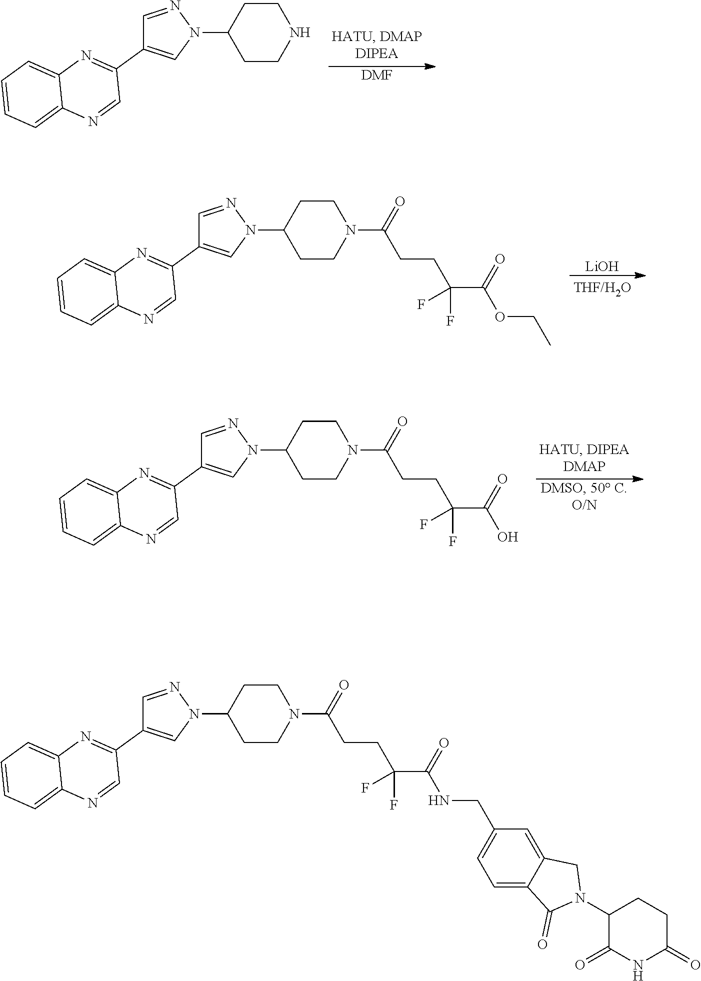

Step 1. Synthesis of ethyl 2,2-difluoro-5-oxo-5-(4-(4-(quinoxalin-2-yl)-1H-pyrazol-1-yl)piperidin-1-yl)pentanoate

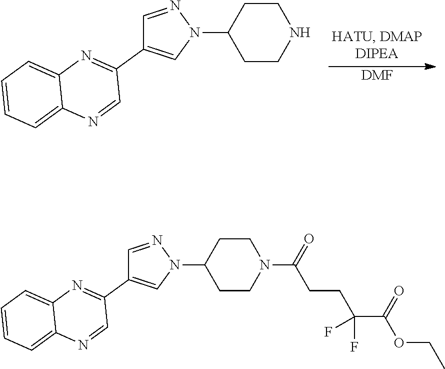

To a mixture of 2-(1-(piperidin-4-yl)-1H-pyrazol-4-yl)quinoxaline (see step 2 of GS-005 for synthesis, 200 mg, 715 μmol) and 5-ethoxy-4,4-difluoro-5-oxo-pentanoic acid (210 mg, 1 mmol) in DMF (5 mL) was added HATU (544 mg, 1.4 mmol), DMAP (8 mg, 71 μmol) and DIPEA (277 mg, 2 mmol). The reaction mixture was stirred at rt overnight. The reaction was purified by reverse phase chromatography (0-70% MeCN in H₂O) to provide the title compound (280 mg, yield: 85%) as light solid. MS (ESI) m/z=458.3 [M+H]⁺.

Step 2. Synthesis of 2,2-difluoro-5-oxo-5-(4-(4-(quinoxalin-2-yl)-1H-pyrazol-1-yl)piperidin-1-yl)pentanoic acid

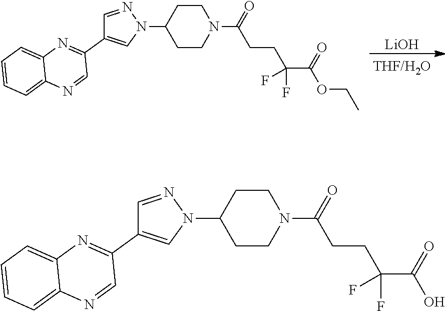 [SMILES: CCOC(=O)C(F)(F)CCC(=O)N1CCC(n2cc(-c3cnc4ccccc4n3)cn2)CC1 ; "LiOH" ; O=C(CCC(F)(F)C(=O)O)N1CCC(n2cc(-c3cnc4ccccc4n3)cn2)CC1]

To a solution of ethyl 2,2-difluoro-5-oxo-5-(4-(4-(quinoxalin-2-yl)-1H-pyrazol-1-yl)piperidin-1-yl)pentanoate (280 mg, 612 μmol) in water (1 mL) and THF (6 mL) was added LiOH (73 mg, 3 mmol). The reaction was stirred at rt for 2 h. The mixture was purified by reverse phase chromatography (0-70% MeCN in H₂O) to provide the title compound (200 mg, yield: 76%) as white solid. MS (ESI) m/z=430.2 [M+H]⁺.

Step 3. Synthesis of N-((2-(2,6-dioxopiperidin-3-yl)-1-oxoisoindolin-5-yl)methyl)-2,2-difluoro-5-oxo-5-(4-(4-(quinoxalin-2-yl)-1H-pyrazol-1-yl)piperidin-1-yl)pentanamide

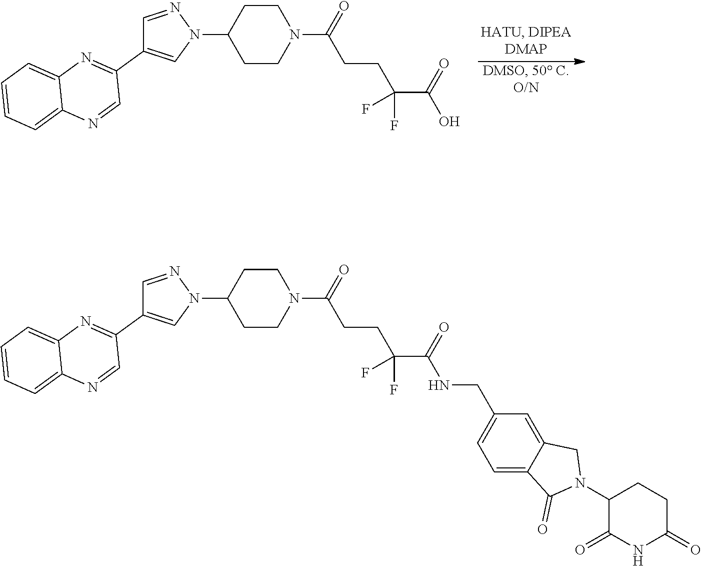 [SMILES: O=C(CCC(F)(F)C(=O)O)N1CCC(n2cc(-c3cnc4ccccc4n3)cn2)CC1 ; "DMSO, 50° C.O/N" ; O=C1CCC(N2Cc3cc(CNC(=O)C(F)(F)CCC(=O)N4CCC(n5cc(-c6cnc7ccccc7n6)cn5)CC4)ccc3C2=O)C(=O)N1]

To a mixture of 2,2-difluoro-5-oxo-5-(4-(4-(quinoxalin-2-yl)-1H-pyrazol-1-yl)piperidin-1-yl)pentanoic acid (200 mg, 465 μmol) and 3-(5-(aminomethyl)-1-oxoisoindolin-2-yl)piperidine-2,6-dione (140 mg, 512 μmol) in DMSO (5 mL) was added HATU (354 mg, 931 μmol), DMAP (6 mg, 46 μmol) and DIPEA (180 mg, 1.4 mmol). The reaction mixture was stirred at 50° C. overnight. The reaction was purified by reverse phase chromatography (0-70% MeCN in H₂O) to provide the title compound (39.4 mg, yield: 12%) as white solid. MS (ESI) m/z=685.5 [M+H]⁺.

### Example 492 and 493. 2-(2,6-Dioxopiperidin-3-yl)-5-((6-(3-methyl-4-(quinoxalin-2-yl)-1H-pyrazol-1-yl)hexyl)amino)isoindoline-1,3-dione (GS-534) and 2-(2,6-dioxopiperidin-3-yl)-5-((6-(5-methyl-4-(quinoxalin-2-yl)-1H-pyrazol-1-yl)hexyl)amino)isoindoline-1,3-dione (GS-535)

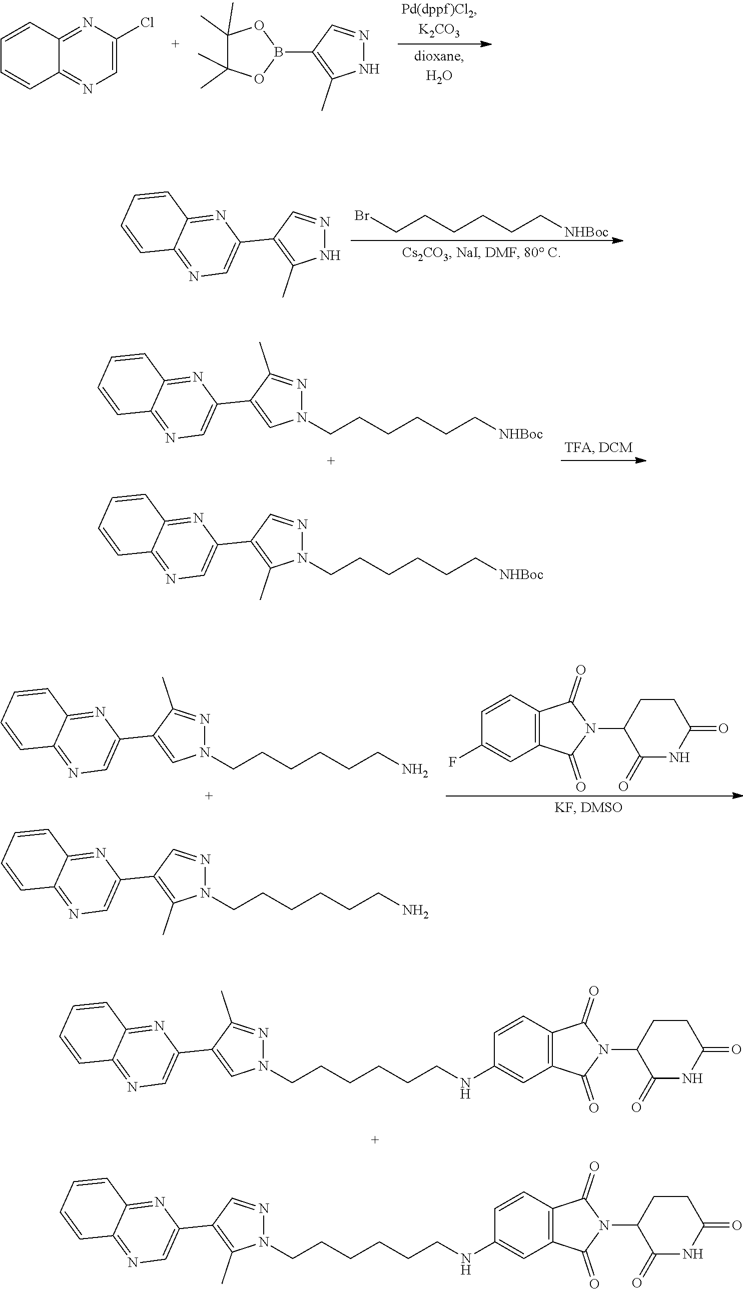

GS-534 and GS-535 were synthesized following the similar procedures as described for GS-540 (GS-534, 3.52 mg, MS (ESI) m/z=566.2 [M+H]⁺; GS-535, 2.62 mg, MS (ESI) m/z=566.2 [M+H]⁺).

### Example 494 and 495. 2-(2,6-Dioxopiperidin-3-yl)-5-((6-(3-(quinoxalin-2-yl)-1H-pyrazol-1-yl)hexyl)amino)isoindoline-1,3-dione (GS-536) and 2-(2,6-dioxopiperidin-3-yl)-5-((6-(5-(quinoxalin-2-yl)-1H-pyrazol-1-yl)hexyl)amino)isoindoline-1,3-dione (GS-537)

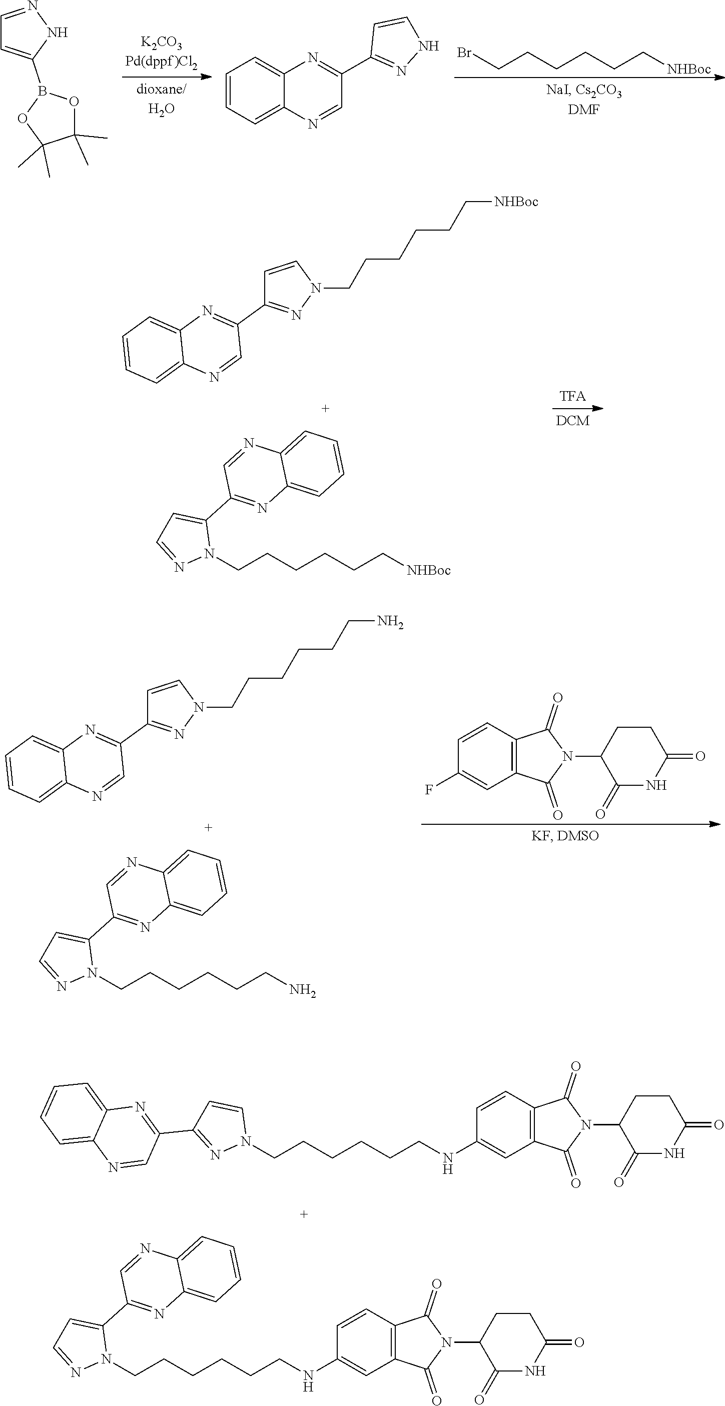

GS-536 and GS-537 were synthesized following the similar procedures as described or GS-540 (GS-536, 14.6 mg, yellow solid, MS (ESI) m/z=552.4 [M+H]L; GS-537, 1.0 mg, yellow solid, MS (ESI) m/z=552.4 [M+H]⁺).

### Example 496. 2-(2,6-Dioxopiperidin-3-yl)-5-(((3-(4-(quinoxalin-2-yl)-1H-pyrazol-1-yl)cyclobutyl)methyl)amino)isoindoline-1,3-dione (GS-538)

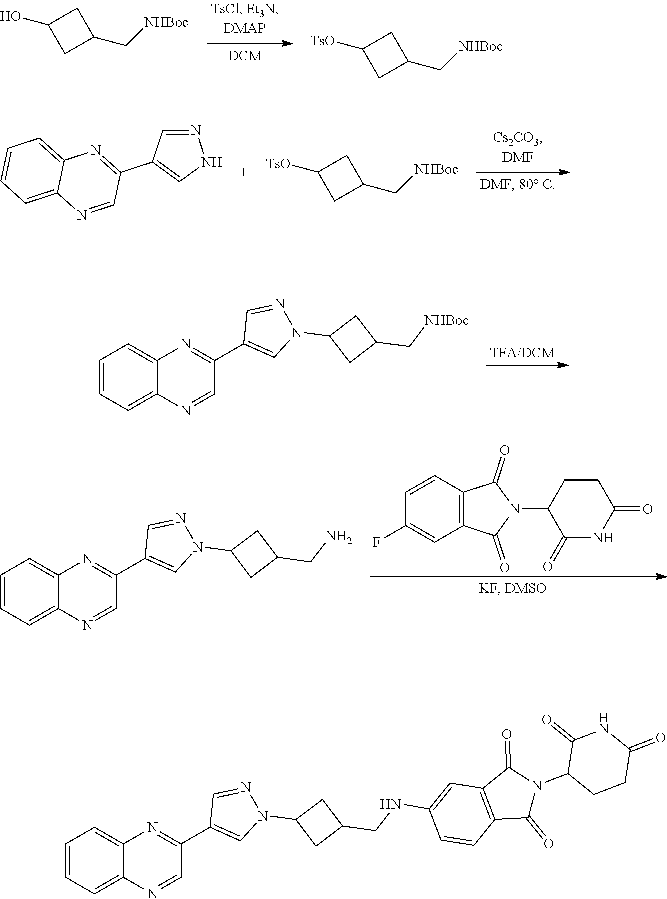

GS-538 was synthesized following the similar procedures as described for GS-524 (2.78 mg). MS (ESI) m/z=536.3 [M+H]⁺.

### Example 497. 2-(2,6-Dioxopiperidin-3-yl)-5-(((6-(4-(quinoxalin-2-yl)-1H-pyrazol-1-yl)spiro[3.3]heptan-2-yl)methyl)amino)isoindoline-1,3-dione (GS-539)

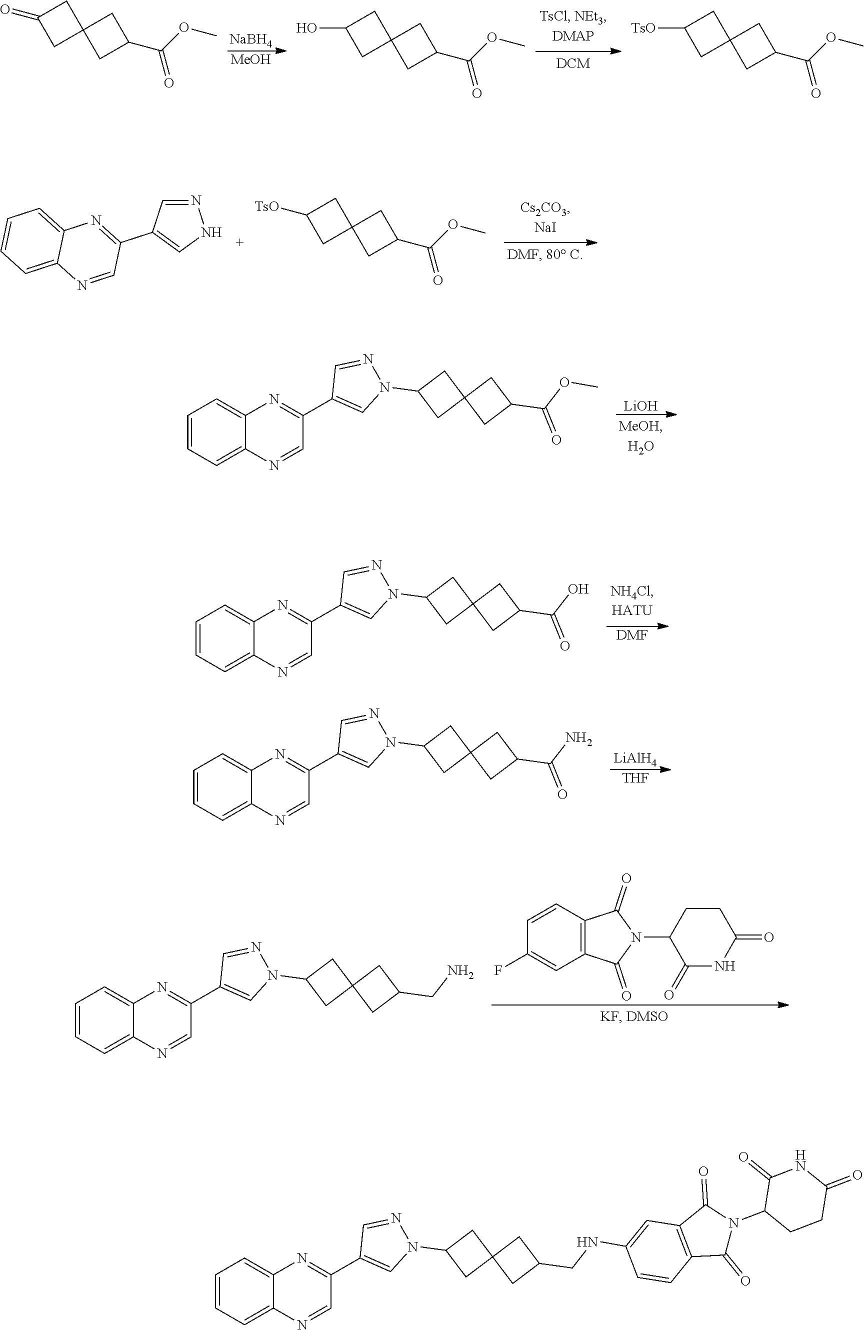

Step 1. Synthesis of methyl 6-hydroxyspiro[3.3]heptane-2-carboxylate

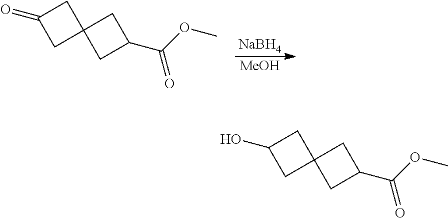

A solution of methyl 2-oxospiro[3.3]heptane-6-carboxylate (200 mg, 1.19 mmol) in methanol (3 mL) was stirred at 0° C. NaBH₄ (67.48 mg, 1.78 mmol) was added slowly. Then, the mixture was concentrated, and the resulting residue was purified by silica gel chromatography to provide the title compound (200 mg, yield: 99%).

Step 2. Synthesis of methyl 6-(tosyloxy)spiro[3.3]heptane-2-carboxylate

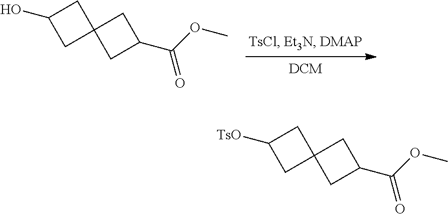 [SMILES: COC(=O)C1CC2(CC(O)C2)C1 ; COC(=O)C1CC2(CC(OS(=O)(=O)c3ccc(C)cc3)C2)C1]

A solution of methyl 2-hydroxyspiro[3.3]heptane-6-carboxylate (200 mg, 1.18 mmol), 4-methylbenzenesulfonyl chloride (336.03 mg, 1.76 mmol), DMAP (14.36 mg, 117.51 μmol) and Et₃N (237.81 mg, 2.35 mmol) in DCM (3 mL) was stirred at 0° C. The mixture was stirred at rt overnight. Then, the mixture was concentrated, and the resulting residue was purified by silica gel chromatography to provide the title compound (320 mg, yield: 84%).

Step 3. Synthesis of methyl 6-(4-(quinoxalin-2-yl)-1H-pyrazol-1-yl)spiro[3.3]heptane-2-carboxylate

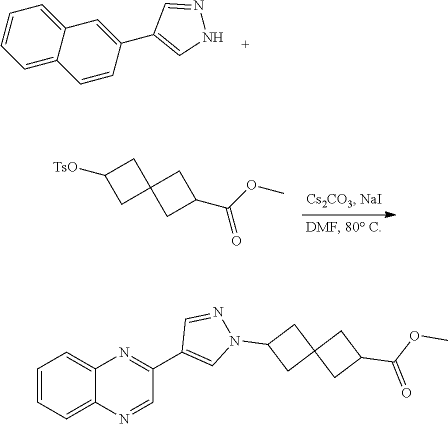 [SMILES: c1ccc2cc(-c3cn[nH]c3)ccc2c1 ; COC(=O)C1CC2(CC(OS(=O)(=O)c3ccc(C)cc3)C2)C1 ; COC(=O)C1CC2(C1)CC(n1cc(-c3cnc4ccccc4n3)cn1)C2]

A solution of 2-(1H-pyrazol-4-yl)quinoxaline (100 mg, 509.66 μmol), methyl 2-(p-tolylsulfonyloxy)spiro[3.3]heptane-6-carboxylate (214.93 mg, 662.56 μmol), K₂CO₃ (140.88 mg, 1.02 mmol), NaI (76.40 mg, 509.66 μmol) in DMF (1 mL) was stirred at 70° C. under N₂ for 2 h. Then, the mixture was concentrated, and the resulting residue was purified by reverse phase chromatography to provide the title compound (160 mg, yield: 90%). MS (ESI) m/z=349.2 [M+H]⁺.

Step 4. Synthesis of 6-(4-(quinoxalin-2-yl)-1H-pyrazol-1-yl)spiro[3.3]heptane-2-carboxylic acid

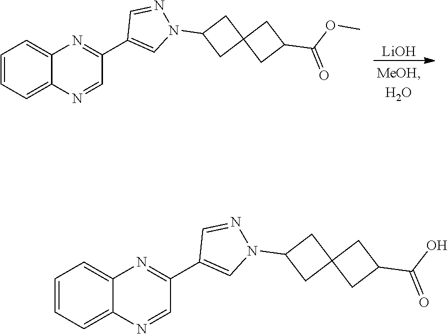

A solution of methyl 2-(4-quinoxaline-2-ylpyrazol-1-yl)spiro[3.3]heptane-6-carboxylate (160 mg, 459.25 μmol), LiOH·H₂O (38.54 mg, 918.49 μmol) in water (1 mL) and methanol (4 mL) was stirred at rt for 1 h. Then, the mixture was concentrated, and the resulting residue was purified by reverse phase chromatography to provide the title compound (130 mg, yield: 85%). MS (ESI) m/z=335.2 [M+H]⁺.

Step 5. Synthesis of 6-(4-(quinoxalin-2-yl)-1H-pyrazol-1-yl)spiro[3.3]heptane-2-carboxamide

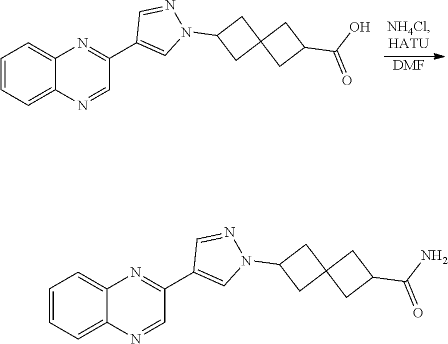

A solution of 2-(4-quinoxalin-2-ylpyrazol-1-yl)spiro[3.3]heptane-6-carboxylic acid (130 mg, 388.79 μmol), NH₄Cl (83.19 mg, 1.56 mmol) and HATU (221.74 mg, 583.18 μmol) in DMF (1 mL) was stirred at rt overnight. Then, the mixture was purified by reverse phase chromatography to provide the title compound (100 mg, yield: 77%). MS (ESI) m/z=334.2 [M+H]⁺.

Step 6. Synthesis of (6-(4-(quinoxalin-2-yl)-1H-pyrazol-1-yl)spiro[3.3]heptan-2-yl)methanamine

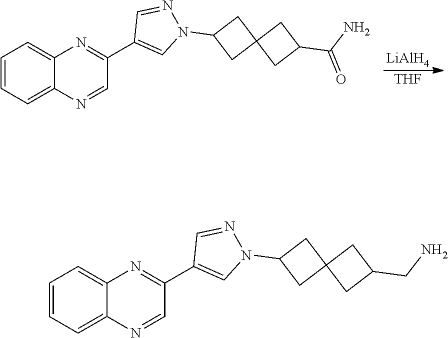 [SMILES: NC(=O)C1CC2(C1)CC(n1cc(-c3cnc4ccccc4n3)cn1)C2 ; "LiAlH4" ; NCC1CC2(C1)CC(n1cc(-c3cnc4ccccc4n3)cn1)C2 ; "THF"]

A solution of 2-(4-quinoxalin-2-ylpyrazol-1-yl)spiro[3.3]heptane-6-carboxamide (100 mg, 299.95 μmol) in THF (2 mL) was stirred at 0° C. under N₂. LiAlH₄ (22.77 mg, 599.90 μmol) was added slowly. The mixture was stirred at rt for 1 h. Then, H₂O was added. The organic phase was separated, and then concentrated. The resulting residue was purified by silica gel chromatography to provide the title compound (15 mg, yield: 16%). MS (ESI) m/z=320.3 [M+H]⁺.

Step 7. Synthesis of 2-(2,6-dioxopiperidin-3-yl)-5-(((6-(4-(quinoxalin-2-yl)-1H-pyrazol-1-yl)spiro[3.3]heptan-2-yl)methyl)amino)isoindoline-1,3-dione

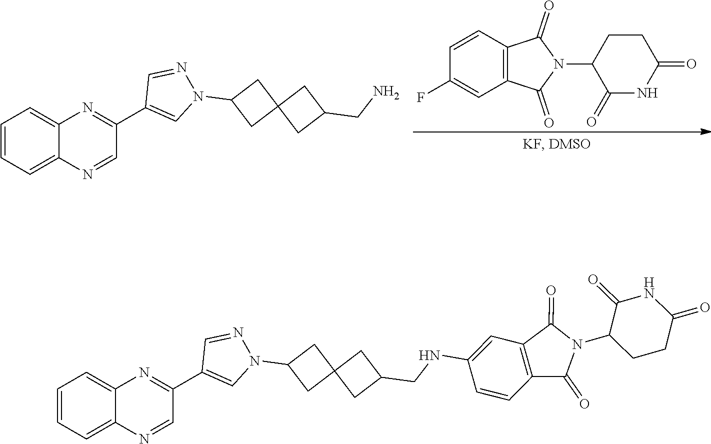

A solution of (6-(4-(quinoxalin-2-yl)-1H-pyrazol-1-yl)spiro[3.3]heptan-2-yl)methanamine (15 mg, 46.96 μmol), 2-(2,6-dioxo-3-piperidyl)-5-fluoro-isoindoline-1,3-dione (19.46 mg, 70.44 μmol) and KF (5.46 mg, 93.93 μmol) in DMSO (1 mL) was stirred at 130° C. under microwave irradiation for 15 min. Then, the mixture was purified by reverse phase chromatography to provide the title compound (1.1 mg, yield: 4%). MS (ESI) m/z=576.4 [M+H]⁺.

### Example 498. 5-((6-(3,5-Dimethyl-4-(3-(trifluoromethyl)quinoxalin-2-yl)-1H-pyrazol-1-yl)hexyl)amino)-2-(2,6-dioxopiperidin-3-yl)isoindoline-1,3-dione (GS-540)

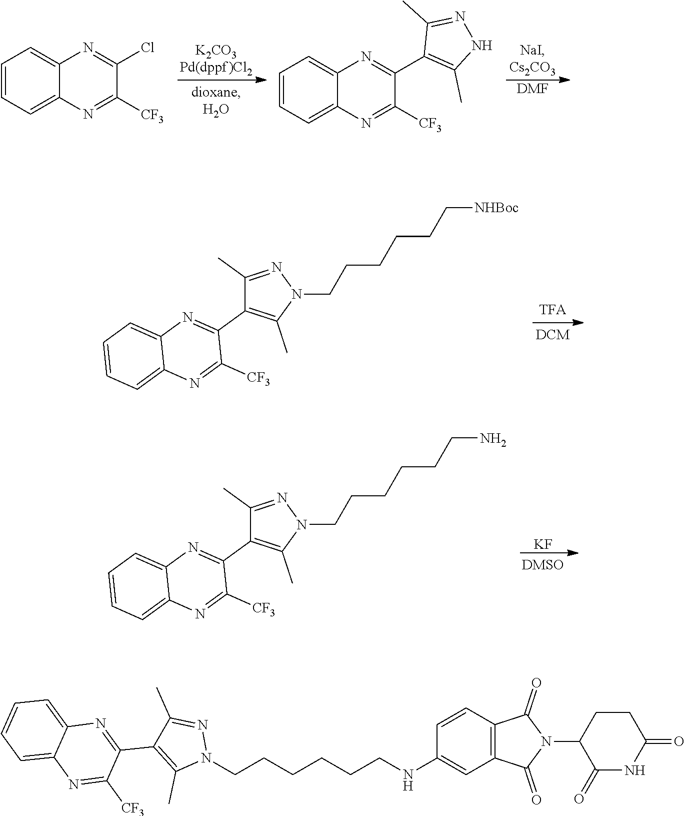

Step 1. Synthesis of 2-(3,5-dimethyl-1H-pyrazol-4-yl)-3-(trifluoromethyl)quinoxaline

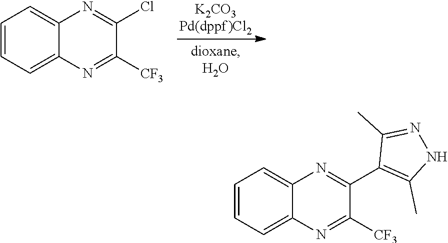 [SMILES: FC(F)(F)c1nc2ccccc2nc1Cl ; Cc1n[nH]c(C)c1-c1nc2ccccc2nc1C(F)(F)F]

To a mixture of 2-chloro-3-(trifluoromethyl)quinoxaline (100 mg, 430 μmol) and 3,5-dimethyl-4-(4,4,5,5-tetramethyl-1,3,2-dioxaborolan-2-yl)-1H-pyrazole (114 mg, 516 μmol) in dioxane (4 mL) and water (1 mL) were added K₂CO₃ (118 mg, 860 μmol) and Pd(dppf)Cl₂ (31 mg, 43 μmol). The reaction mixture was stirred at 100° C. for 2 h under N₂. The solution was quenched with water, and extracted with EtOAc. The organic phase was washed with water, dried over Na₂SO₄, filtered, and concentrated. The resulting residue was purified by reverse phase chromatography (0-70% MeCN in H₂O) to provide the title compound (70 mg, yield: 55%) as white solid. MS (ESI) m/z=293.1 [M+H]⁺.

Step 2. Synthesis of tert-butyl (6-(3,5-dimethyl-4-(3-(trifluoromethyl)quinoxalin-2-yl)-1H-pyrazol-1-yl)hexyl)carbamate

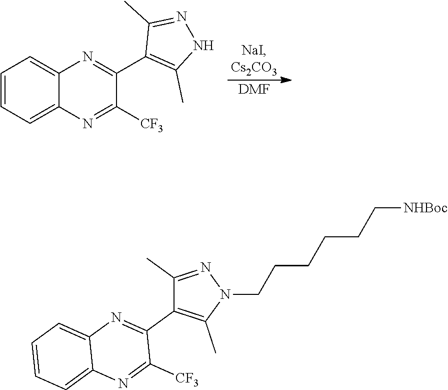

To a mixture of 2-(3,5-dimethyl-1H-pyrazol-4-yl)-3-(trifluoromethyl)quinoxaline (70 mg, 239 mol) and tert-butyl N-(6-bromohexyl)carbamate (67 mg, 239 μmol) in DMF (4 mL) were added Cs₂CO₃ (156 mg, 479 μmol) and NaI (43 mg, 287 μmol). The reaction mixture was stirred at 100° C. for 1 h. The reaction was quenched with water and extracted with EtOAc. The organic phase was washed with water, dried over Na₂SO₄, filtered, and concentrated. The resulting residue was purified by reverse phase chromatography (0-70% MeCN in H₂O) to provide the title compound (100 mg, yield: 85%) as white solid. MS (ESI) m/z=492.3 [M+H]⁺.

Step 3. Synthesis of 6-(3,5-dimethyl-4-(3-(trifluoromethyl)quinoxalin-2-yl)-1H-pyrazol-1-yl)hexan-1-amine

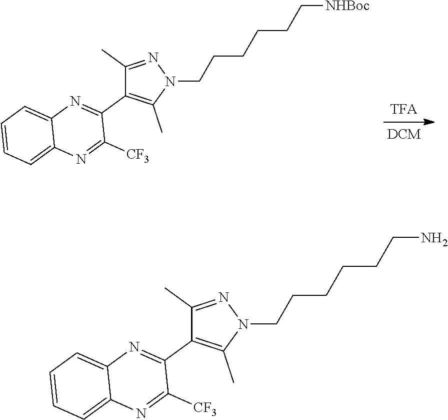 [SMILES: Cc1nn(CCCCCCNC(=O)OC(C)(C)C)c(C)c1-c1nc2ccccc2nc1C(F)(F)F ; Cc1nn(CCCCCCN)c(C)c1-c1nc2ccccc2nc1C(F)(F)F]

To a solution of tert-butyl (6-(3,5-dimethyl-4-(3-(trifluoromethyl)quinoxalin-2-yl)-1H-pyrazol-1-yl)hexyl)carbamate (100 mg, 203 μmol) in DCM (2 mL) was added TFA (1 mL). The reaction mixture was stirred at rt for 20 min. After concentration, the mixture was purified by reverse phase chromatography (0-70% MeCN in H₂O) to provide the title compound (70 mg, yield: 88%) as bright oil. MS (ESI) m/z=392.3 [M+H]⁺.

Step 4. Synthesis of 5-((6-(3,5-dimethyl-4-(3-(trifluoromethyl)quinoxalin-2-yl)-1H-pyrazol-1-yl)hexyl)amino)-2-(2,6-dioxopiperidin-3-yl)isoindoline-1,3-dione

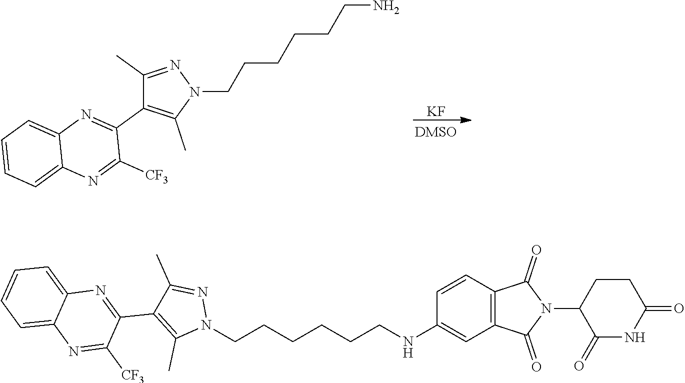 [SMILES: Cc1nn(CCCCCCN)c(C)c1-c1nc2ccccc2nc1C(F)(F)F ; "KF" ; Cc1nn(CCCCCCNc2ccc3c(c2)C(=O)N(C2CCC(=O)NC2=O)C3=O)c(C)c1-c1nc2ccccc2nc1C(F)(F)F]

To a mixture of 6-(3,5-dimethyl-4-(3-(trifluoromethyl)quinoxalin-2-yl)-1H-pyrazol-1-yl)hexan-1-amine (70 mg, 178 μmol) and 2-(2,6-dioxo-3-piperidyl)-5-fluoro-isoindoline-1,3-dione (54 mg, 197 μmol) in DMSO (2 mL) was added KF (10 mg, 179 μmol). The reaction mixture was irradiated at 135° C. in the microwave reactor for 15 min. The reaction was purified by reverse phase chromatography (0-70% MeCN in H₂O) to provide the title compound (17.4 mg, yield: 15%) as yellow solid. MS (ESI) m/z=648.5 [M+H]⁺.

### Example 499. 2-(2,6-Dioxopiperidin-3-yl)-5-((6-(4-(7-(piperidin-4-yl)quinoxalin-2-yl)-1H-pyrazol-1-yl)hexyl)amino)isoindoline-1,3-dione (GS-541)

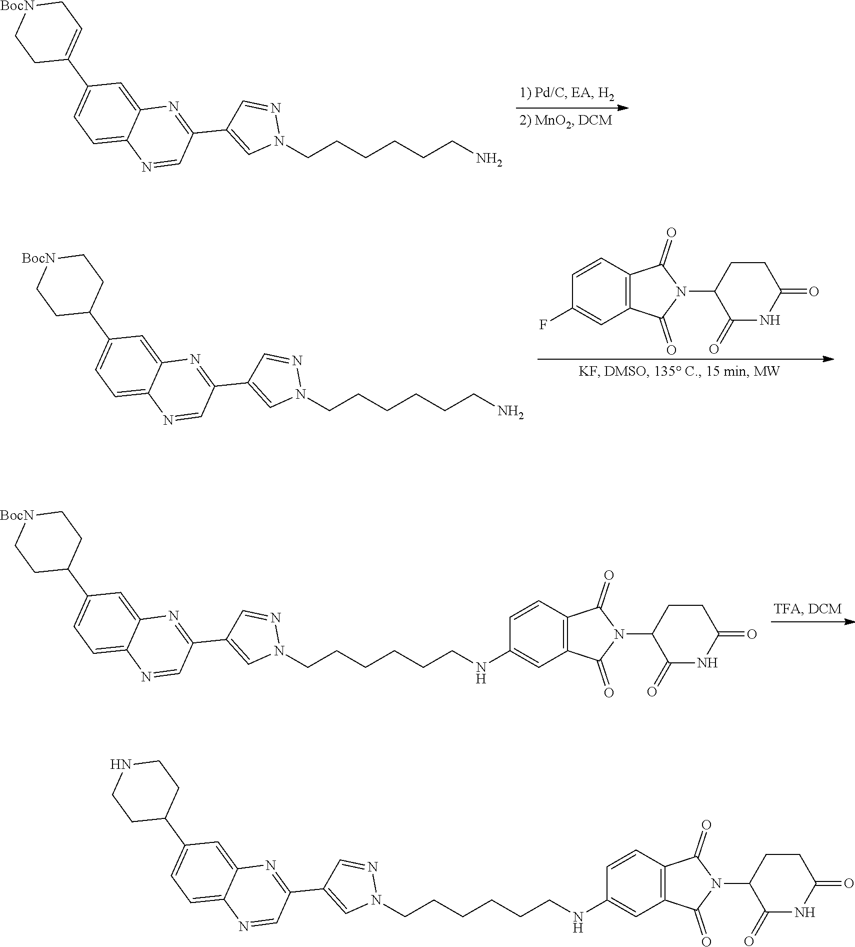 [SMILES: CC(C)(C)OC(=O)N1CC=C(c2ccc3ncc(-c4cnn(CCCCCCN)c4)nc3c2)CC1 ; CC(C)(C)OC(=O)N1CCC(c2ccc3ncc(-c4cnn(CCCCCCN)c4)nc3c2)CC1 ; O=C1CCC(N2C(=O)c3ccc(F)cc3C2=O)C(=O)N1 ; CC(C)(C)OC(=O)N1CCC(c2ccc3ncc(-c4cnn(CCCCCCNc5ccc6c(c5)C(=O)N(C5CCC(=O)NC5=O)C6=O)c4)nc3c2)CC1 ; O=C1CCC(N2C(=O)c3ccc(NCCCCCCn4cc(-c5cnc6ccc(C7CCNCC7)cc6n5)cn4)cc3C2=O)C(=O)N1]

Step 1. Synthesis of tert-butyl 4-(3-(1-(6-aminohexyl)-1H-pyrazol-4-yl)quinoxalin-6-yl)piperidine-1-carboxylate

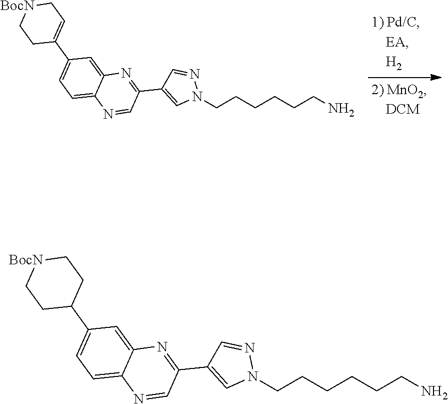 [SMILES: CC(C)(C)OC(=O)N1CC=C(c2ccc3ncc(-c4cnn(CCCCCCN)c4)nc3c2)CC1 ; CC(C)(C)OC(=O)N1CCC(c2ccc3ncc(-c4cnn(CCCCCCN)c4)nc3c2)CC1]

To a solution of tert-butyl 4-(3-(1-(6-aminohexyl)-1H-pyrazol-4-yl)quinoxalin-6-yl)-3,6-dihydropyridine-1(2H)-carboxylate (see step 1 of GS-531 for synthesis, 134 mg, 0.28 mmol) in EtOAc (10 mL) was added Pd/C (200 mg). The mixture was stirred at rt for 16 h under H₂. After filtration, the filtrate was concentrated. To a solution of the residue in DCM (5 mL) was added MnO₂ (50 mg). After being stirred at rt for 20 min, the mixture was filtered and concentrated to provide the title compound, which was used directly without further purification. MS (ESI) m/z=479.4 [M+H]⁺.

Step 2. Synthesis of tert-butyl 4-(3-(1-(6-((2-(2,6-dioxopiperidin-3-yl)-1,3-dioxoisoindolin-5-yl)amino)hexyl)-1H-pyrazol-4-yl)quinoxalin-6-yl)piperidine-1-carboxylate

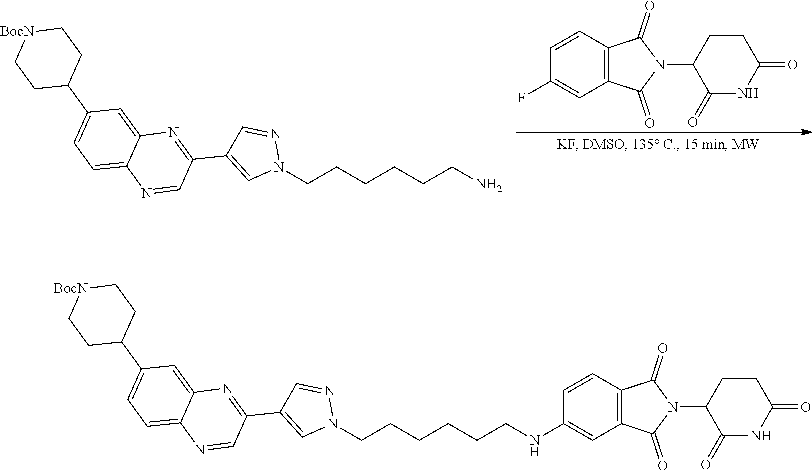 [SMILES: CC(C)(C)OC(=O)N1CCC(c2ccc3ncc(-c4cnn(CCCCCCN)c4)nc3c2)CC1 ; O=C1CCC(N2C(=O)c3ccc(F)cc3C2=O)C(=O)N1 ; CC(C)(C)OC(=O)N1CCC(c2ccc3ncc(-c4cnn(CCCCCCNc5ccc6c(c5)C(=O)N(C5CCC(=O)NC5=O)C6=O)c4)nc3c2)CC1]

To a solution of tert-butyl 4-(3-(1-(6-aminohexyl)-1H-pyrazol-4-yl)quinoxalin-6-yl)piperidine-1-carboxylate (20 mg, 0.04 mmol) in DMSO (1 mL) were added 2-(2,6-dioxopiperidin-3-yl)-5-fluoroisoindoline-1,3-dione (22.1 mg, 0.08 mmol) and KF (7.2 mg, 0.012 mmol). After the mixture was irradiated at 135° C. in the microwave reactor for 15 min, it was purified by reverse phase chromatography (MeOH:H₂O=1:1) to provide the title compound (9 mg, yield: 29%) as yellow oil. MS (ESI) m/z=735.4 [M+H]⁺.

Step 3. Synthesis of 2-(2,6-dioxopiperidin-3-yl)-5-((6-(4-(7-(piperidin-4-yl)quinoxalin-2-yl)-1H-pyrazol-1-yl)hexyl)amino)isoindoline-1,3-dione

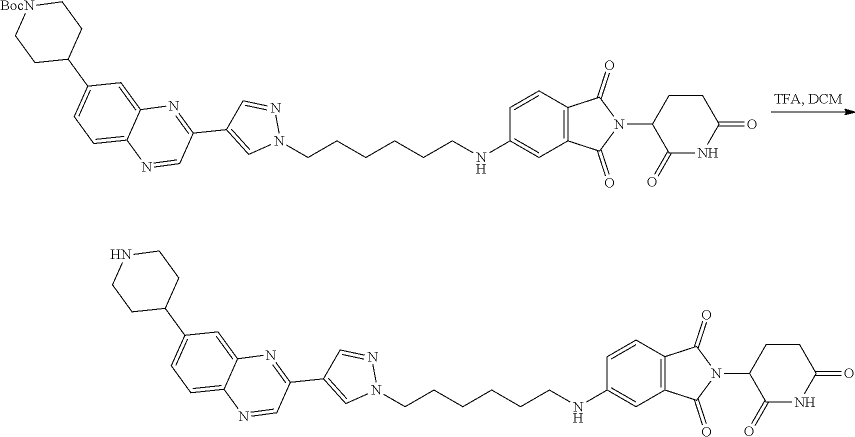 [SMILES: CC(C)(C)OC(=O)N1CCC(c2ccc3ncc(-c4cnn(CCCCCCNc5ccc6c(c5)C(=O)N(C5CCC(=O)NC5=O)C6=O)c4)nc3c2)CC1 ; O=C1CCC(N2C(=O)c3ccc(NCCCCCCn4cc(-c5cnc6ccc(C7CCNCC7)cc6n5)cn4)cc3C2=O)C(=O)N1]

To a solution of tert-butyl 4-(3-(1-(6-((2-(2,6-dioxopiperidin-3-yl)-1,3-dioxoisoindolin-5-yl)amino)hexyl)-1H-pyrazol-4-yl)quinoxalin-6-yl)piperidine-1-carboxylate (9 mg, 0.01 mmol) in DCM (1 mL) was added TFA (1 mL). The mixture was stirred at rt for 30 min, before it was purified by reverse phase chromatography (MeOH:H₂O=1:1) to provide the title compound (3 mg, yield: 39%) as yellow solid. MS (ESI) m/z=635.4 [M+H]⁺.

### Example 500. 2-(2,6-Dioxopiperidin-3-yl)-5-((6-(4-(7-(tetrahydro-2H-pyran-4-yl)quinoxalin-2-yl)-1H-pyrazol-1-yl)hexyl)amino)isoindoline-1,3-dione (GS-542)

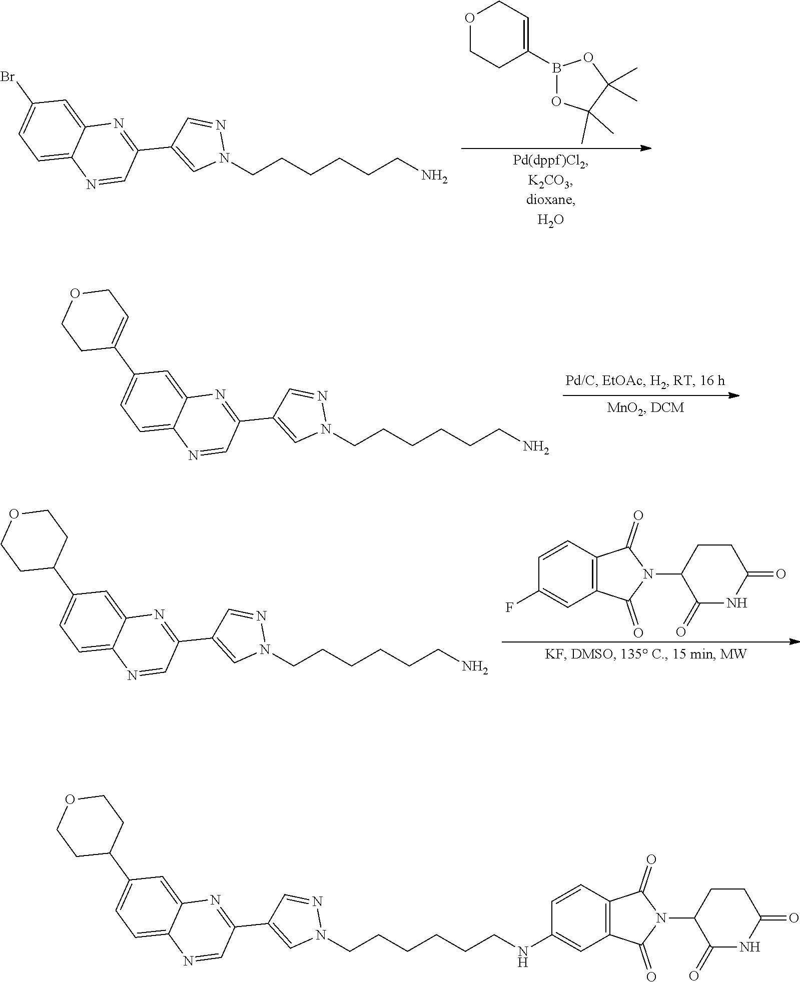 [SMILES: NCCCCCCn1cc(-c2cnc3ccc(Br)cc3n2)cn1 ; CC1(C)OB(C2=CCOCC2)OC1(C)C ; NCCCCCCn1cc(-c2cnc3ccc(C4=CCOCC4)cc3n2)cn1 ; NCCCCCCn1cc(-c2cnc3ccc(C4CCOCC4)cc3n2)cn1 ; O=C1CCC(N2C(=O)c3ccc(F)cc3C2=O)C(=O)N1 ; O=C1CCC(N2C(=O)c3ccc(NCCCCCCn4cc(-c5cnc6ccc(C7CCOCC7)cc6n5)cn4)cc3C2=O)C(=O)N1]

GS-542 was synthesized following the similar procedures as described for GS-541 (3 mg) as yellow solid. MS (ESI) m/z=636.4 [M+H]⁺.

### Example 501. 2-(2,6-Dioxopiperidin-3-yl)-5-((5-oxo-5-(4-(4-(7-(piperazin-1-yl)quinoxalin-2-yl)-1H-pyrazol-1-yl)piperidin-1-yl)pentyl)amino)isoindoline-1,3-dione (GS-543)

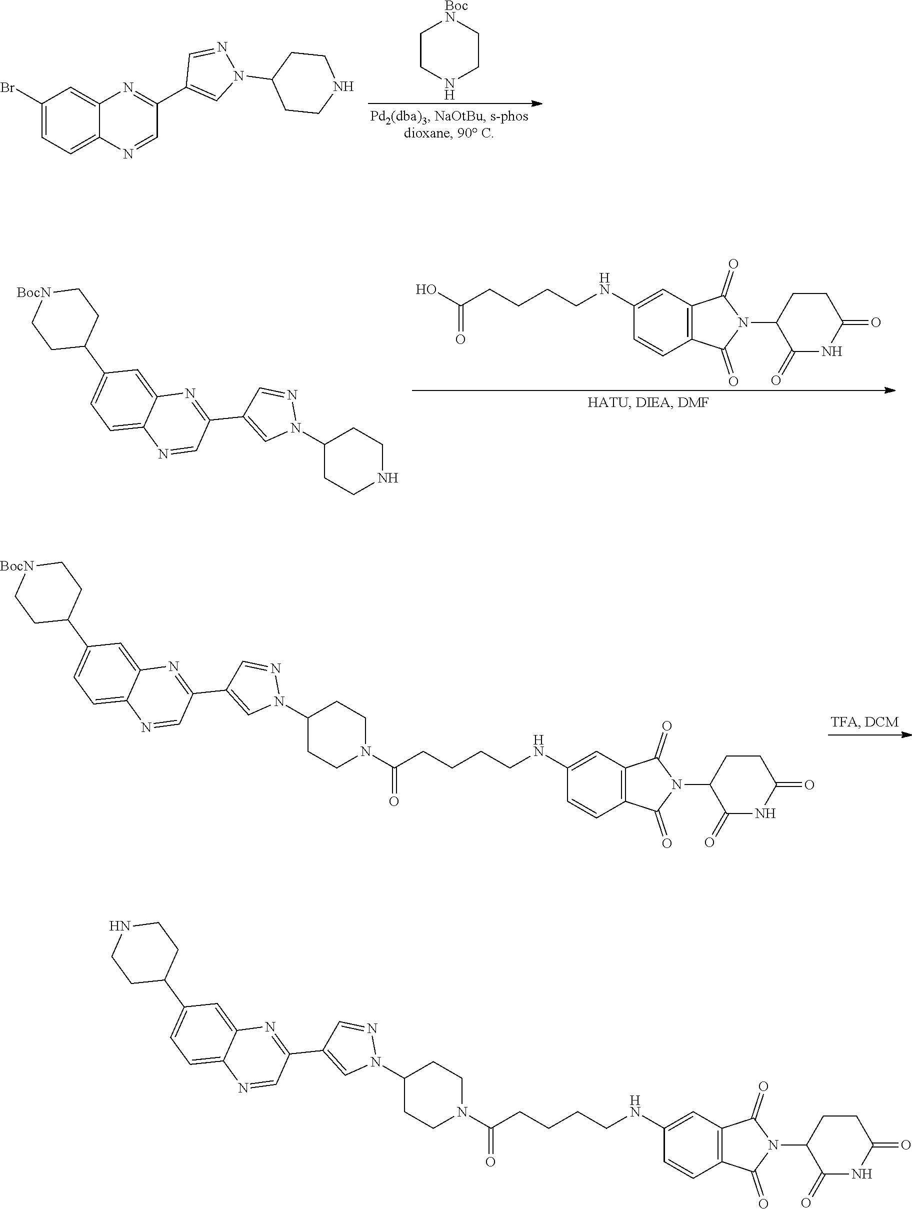 [SMILES: Brc1ccc2ncc(-c3cnn(C4CCNCC4)c3)nc2c1 ; CC(C)(C)OC(=O)N1CCNCC1 ; CC(C)(C)OC(=O)N1CCC(c2ccc3ncc(-c4cnn(C5CCNCC5)c4)nc3c2)CC1 ; O=C(O)CCCCNc1ccc2c(c1)C(=O)N(C1CCC(=O)NC1=O)C2=O ; CC(C)(C)OC(=O)N1CCC(c2ccc3ncc(-c4cnn(C5CCN(C(=O)CCCCNc6ccc7c(c6)C(=O)N(C6CCC(=O)NC6=O)C7=O)CC5)c4)nc3c2)CC1 ; O=C1CCC(N2C(=O)c3ccc(NCCCCC(=O)N4CCC(n5cc(-c6cnc7ccc(C8CCNCC8)cc7n6)cn5)CC4)cc3C2=O)C(=O)N1]

Step 1. Synthesis of tert-butyl 4-(3-(1-(piperidin-4-yl)-1H-pyrazol-4-yl)quinoxalin-6-yl)piperazine-1-carboxylate

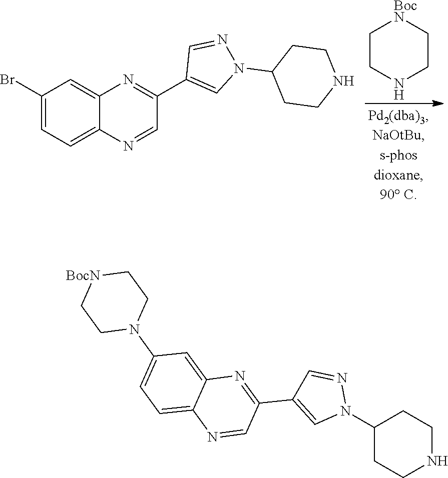

To a solution of 7-bromo-2-(1-(piperidin-4-yl)-1H-pyrazol-4-yl)quinoxaline (see step 2 of GS-525 for synthesis, 100 mg, 279.15 μmol) in dioxane (5 mL) were added tert-butyl piperazine-1-carboxylate (62.39 mg, 334.98 μmol), Pd₂(dba)₃ (25.54 mg, 27.91 μmol), sodium tert-butoxide (53.65 mg, 558.29 μmol) and s-phos (22.95 mg, 55.83 μmol). The mixture was stirred at 90° C. for 1 h, before it was filtered. The filtrate was concentrated and purified by reverse phase chromatography (water:MeOH=1:1) to provide the title compound (101 mg, yield: 78%) as red oil. MS (ESI) m/z=464.3 [M−Boc+H]⁺.

Step 2 to 3. Synthesis of 2-(2,6-dioxopiperidin-3-yl)-5-((5-oxo-5-(4-(4-(7-(piperazin-1-yl)quinoxalin-2-yl)-1H-pyrazol-1-yl)piperidin-1-yl)pentyl)amino)isoindoline-1,3-dione

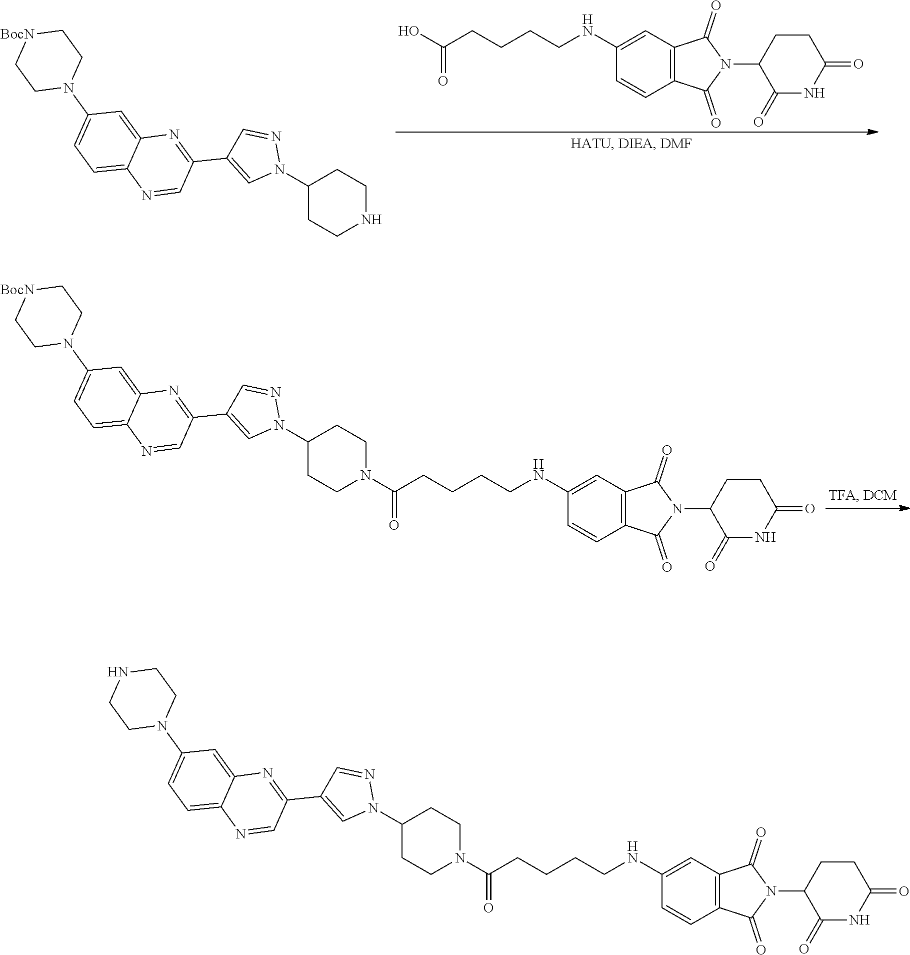 [SMILES: CC(C)(C)OC(=O)N1CCN(c2ccc3ncc(-c4cnn(C5CCNCC5)c4)nc3c2)CC1 ; O=C(O)CCCCNc1ccc2c(c1)C(=O)N(C1CCC(=O)NC1=O)C2=O ; CC(C)(C)OC(=O)N1CCN(c2ccc3ncc(-c4cnn(C5CCN(C(=O)CCCCNc6ccc7c(c6)C(=O)N(C6CCC(=O)NC6=O)C7=O)CC5)c4)nc3c2)CC1 ; O=C1CCC(N2C(=O)c3ccc(NCCCCC(=O)N4CCC(n5cc(-c6cnc7ccc(N8CCNCC8)cc7n6)cn5)CC4)cc3C2=O)C(=O)N1]

GS-543 was synthesized following the similar procedures as described or steps 4 to 5 of GS-525 (4 mg, yield over 2 steps: 26%) as red solid. MS (ESI) m/z=719.4 [M+H]⁺.

### Example 502. 2-(2,6-Dioxopiperidin-3-yl)-5-((5-oxo-5-(4-(4-(7-(1,2,3,6-tetrahydropyridin-4-yl)quinoxalin-2-yl)-1H-pyrazol-1-yl)piperidin-1-yl)pentyl)amino)isoindoline-1,3-dione (GS-544)

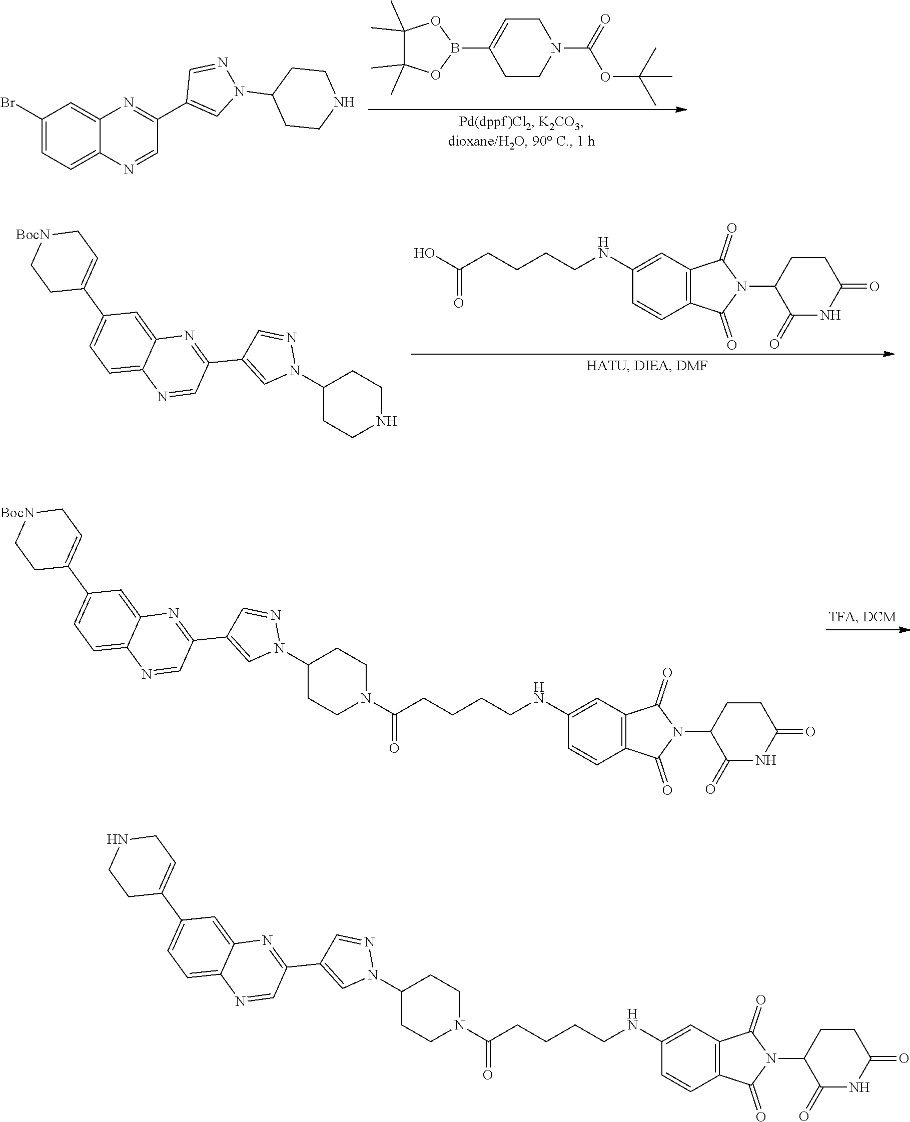

GS-544 was synthesized following the similar procedures as described for steps 3 to 5 of GS-525 (4 mg, yield over 3 steps: 20%) as yellow solid. MS (ESI) m/z=716.4 [M+H]⁺.

### Example 503. 2-(2,6-Dioxopiperidin-3-yl)-5-((5-oxo-5-(4-(quinoxalin-2-yl)piperidin-1-yl)pentyl)amino)isoindoline-1,3-dione (GS-545)

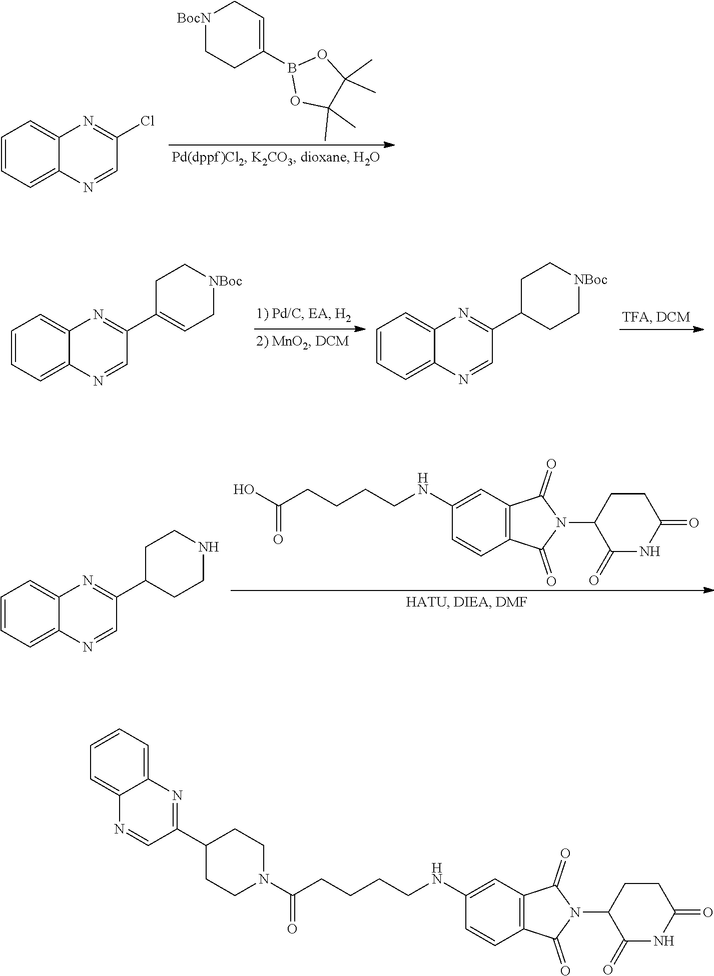

Step 1. Synthesis of tert-butyl 4-(qunioxalin-2-yl)-3,6-dihydropyridine-1-(H-carboxylate

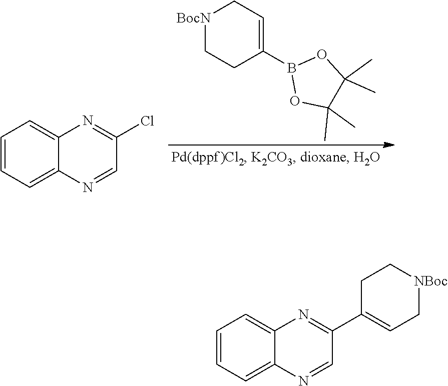 [SMILES: Clc1cnc2ccccc2n1 ; CC(C)(C)OC(=O)N1CC=C(B2OC(C)(C)C(C)(C)O2)CC1 ; CC(C)(C)OC(=O)N1CC=C(c2cnc3ccccc3n2)CC1]

To a solution of 2-chloroquinoxaline (100 mg, 0.61 mmol) in dioxane (2 ml) were added tert-butyl 4-(4,4,5,5-tetramethyl-1,3,2-dioxaborolan-2-yl)-3,6-dihydropyridine-1(2H)-carboxylate (226 mg, 0.73 mmol), Pd(dppf)Cl₂ (22 mg, 0.03 mmol), K₂CO₃ (168 mg, 1.22 mmol) and H₂O (0.4 mL). After the mixture was stirred at 90° C. for 1 h, it was purified by reverse phase chromatography (MeOH:H₂O=2:1) to provide the title compound (162 mg, yield: 85%) as yellow oil. MS (ESI) m/z=312.3 [M+H]⁺.

Step 2. Synthesis of tert-butyl 4-(quinoxalin-2-yl)piperidine-1-carboxylate

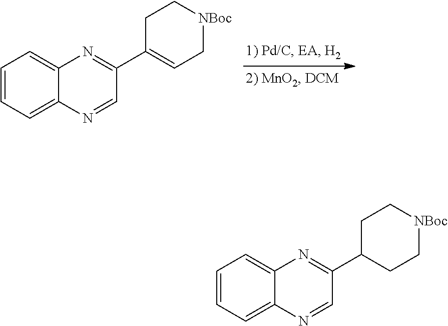 [SMILES: CC(C)(C)OC(=O)N1CC=C(c2cnc3ccccc3n2)CC1 ; CC(C)(C)OC(=O)N1CCC(c2cnc3ccccc3n2)CC1]

To a solution of tert-butyl 4-(quinoxalin-2-yl)-3,6-dihydropyridine-1(2H)-carboxylate (162 mg, 0.52 mmol) in EtOAc (10 mL) was added Pd/C (150 mg). The mixture was purged with H₂, and stirred at rt for 16 h under H₂ atmosphere. The suspension was filtered, and the filtrate was concentrated. To a solution of the above residue in DCM (5 mL) was added MnO₂ (50 mg). After the mixture was stirred at rt for 20 min, it was filtered. The filtrate was concentrated to provide the title compound (116 mg, yield: 77%), which was used directly without further purification. MS (ESI) m/z=314.3 [M+H]⁺.

Step 3. Synthesis of 2-(piperidin-4-yl)quinoxaline

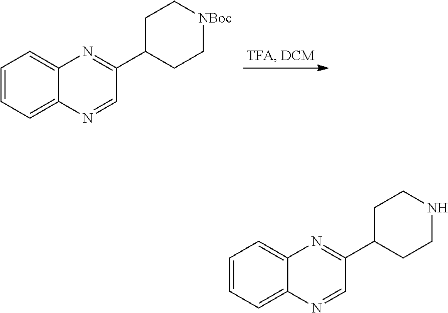 [SMILES: CC(C)(C)OC(=O)N1CCC(c2cnc3ccccc3n2)CC1 ; c1ccc2nc(C3CCNCC3)cnc2c1]

To a solution of tert-butyl 4-quinoxalin-2-ylpiperidine-1-carboxylate (50 mg, 159.54 μmol) in DCM (2 mL) was added TFA (1 mL). The mixture was stirred at rt for 1 h, before it was concentrated to provide the title compound (30 mg, yield: 88%) as yellow oil, which was used directly in the next step. MS (ESI) m/z=214.3 [M+H]⁺.

Step 4. Synthesis of 2-(2,6-dioxopiperidin-3-yl)-5-((5-oxo-5-(4-(quinoxalin-2-yl)piperidin-1-yl)pentyl)amino)isoindoline-1,3-dione

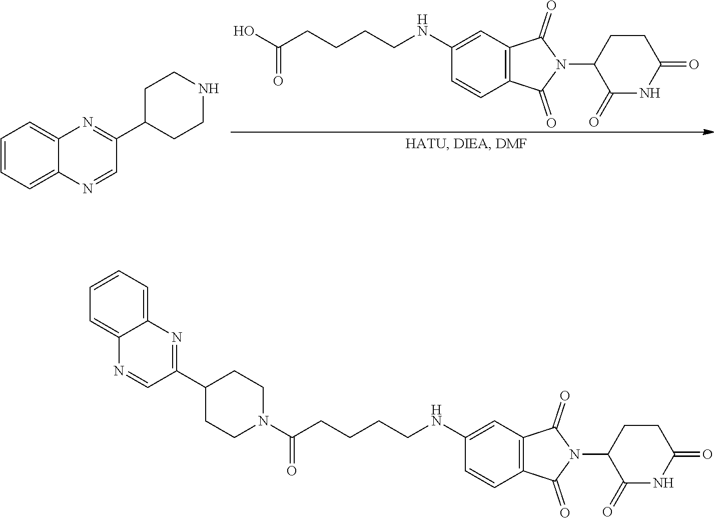 [SMILES: c1ccc2nc(C3CCNCC3)cnc2c1 ; O=C(O)CCCCNc1ccc2c(c1)C(=O)N(C1CCC(=O)NC1=O)C2=O ; O=C1CCC(N2C(=O)c3ccc(NCCCCC(=O)N4CCC(c5cnc6ccccc6n5)CC4)cc3C2=O)C(=O)N1]

To a solution of 2-(piperidin-4-yl)quinoxaline (10 mg, 0.04 mmol) in DMF (1 ml) were added 5-((2-(2,6-dioxopiperidin-3-yl)-1,3-dioxoisoindolin-5-yl)amino)pentanoic acid (16 mg, 0.04 mmol), HATU (22 mg, 0.06 mmol) and DIEA (15 mg, 0.12 mmol). After the mixture was stirred at rt for 3 h, it was purified by reverse phase chromatography (MeOH:H₂O=1:1) to provide the title compound (5 mg, yield: 19%) as yellow solid. MS (ESI) m/z=569.3 [M+H]⁺.

### Example 504. 2-(2,6-Dioxopiperidin-3-yl)-5-((6-(4-methyl-3-(quinoxalin-2-yl)-TH-pyrazol-1-yl)hexyl)amino)isoindoline-1,3-dione (GS-546)

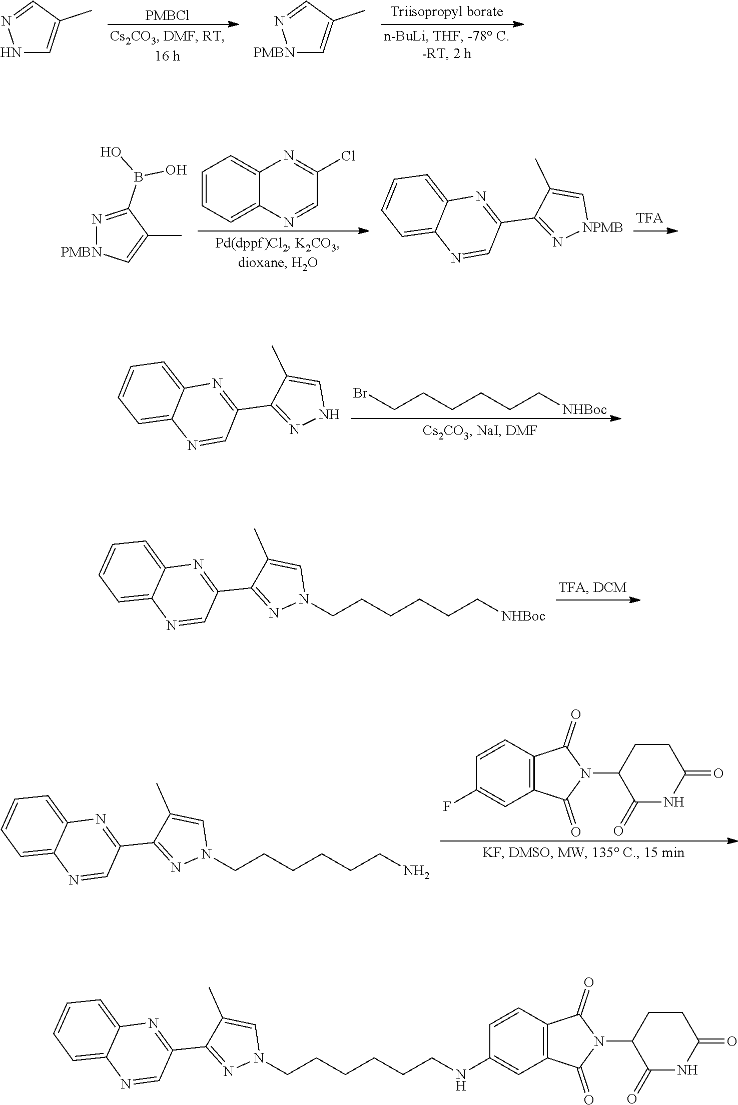

Step 1. Synthesis of 1-(4-methoxybenzyl) 4-methyl-1H-pyrazole

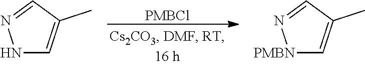

A mixture of 4-methyl-1H-pyrazole (5.00 g, 60.90 mmol), PMBCl (11.44 g, 73.08 mmol) and Cs₂CO₃ (39.68 g, 121.8 mmol) in DMF (50 mL) was stirred at rt for 16 h. The resulting mixture was diluted with H₂O (200 mL) and extracted with EtOAc (200 mL×3). Thecombined organic layers were dried over Na₂SO₄, filtered, and concentrated in vacuo to provide the title compound (12.0 g, yield: 97%) as colorless oil.

Step 2. Synthesis of (1-(4-methoxybenzyl)-4-methyl-1H-pyrazol-3-yl)boronic acid

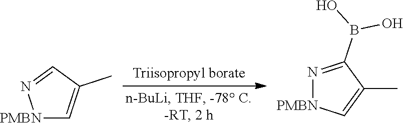

To a solution of 1-(4-methoxybenzyl)-4-methyl-1H-pyrazole (5.26 g, 26.0 mmol) in tetrahydrofuran (50 mL) was added n-butyllithium solution (2.5 M in hexane, 78.0 mmol, 31 mL) dropwise at 0° C. under nitrogen atmosphere. The resulting solution was stirred at rt for 1 h, before it was cooled to −78° C. Triisopropyl borate (14.67 g, 78.017 mmol) was added. And the reaction mixture was stirred at −78° C. for 0.5 h and then at rt for 2 h. The mixture was slowly warmed up to 0° C., before it was quenched with NH₄Cl solution (250 mL), and extracted with EtOAc (250 mL×3). The combined organic layers were washed with brine, dried over Na₂SO₄, and concentrated under reduced pressure. The resulting residue was purified by silica gel chromatography (EtOAc:petroleum ether=1:1) to provide the title compound (3.00 g, yield: 47%) as off-white solid.

Step 3. Synthesis of 2-(1-(4-methoxybenzyl)-4-methyl-1H-pyrazol-3-yl)quinoxaline

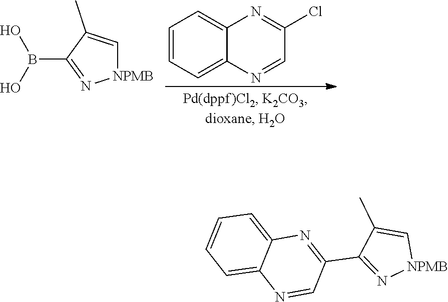

To a solution of (1-(4-methoxybenzyl)-4-methyl-1H-pyrazol-3-yl)boronic acid (150 mg, 0.61 mmol) in dioxane (3 mL) were added 2-chloroquinoxaline (100 mg, 0.61 mmol), Pd(dppf)Cl₂ (22 mg, 0.03 mmol), K₂CO₃ (165 mg, 1.2 mmol) and H₂O (0.5 mL). The mixture was stirred at 90° C. for 2 h, before it was purified by reverse phase chromatography (MeOH:H₂O=2:1) to provide the title compound (158 mg, yield: 79%) as yellow oil. MS (ESI) m/z=331.3 [M+H]⁺.

Step 4. Synthesis of 2-(4-methyl-1H-pyrazol-3-yl)quinoxaline

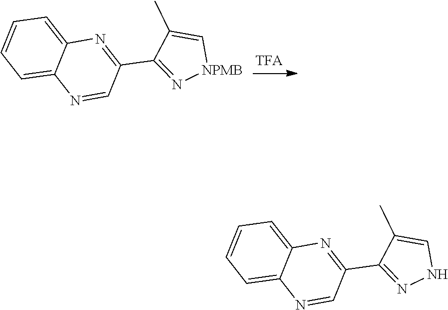

A solution of 2-(1-(4-methoxybenzyl)-4-methyl-1H-pyrazol-3-yl)quinoxaline (150 mg, 454.02 μmol) in TFA (3 mL) was stirred at 50° C. for 16 h. The mixture was concentrated to provide the title compound (80 mg, yield: 84%) as yellow oil, which was used directly in the next step. MS (ESI) m/z=211.3 [M+H]⁺.

Step 5 to 7. Synthesis of 2-(2,6-dioxopiperidin-3-yl)-5-((6-(4-methyl-3-(quinoxalin-2-yl)-1H-pyrazol-1-yl)hexyl)amino)isoindoline-1,3-dione

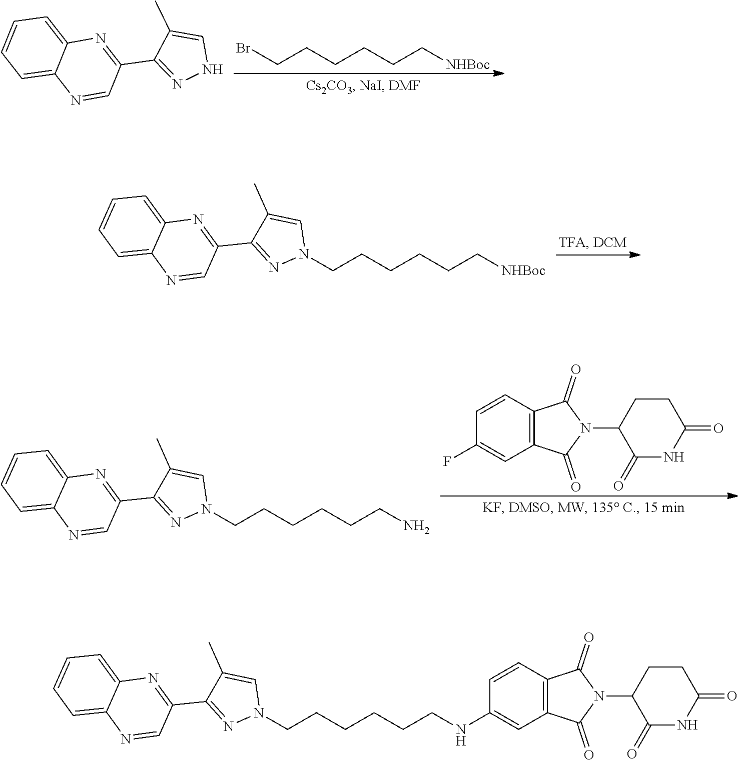 [SMILES: Cc1c[nH]nc1-c1cnc2ccccc2n1 ; CC(C)(C)OC(=O)NCCCCCCBr ; Cc1cn(CCCCCCNC(=O)OC(C)(C)C)nc1-c1cnc2ccccc2n1 ; Cc1cn(CCCCCCN)nc1-c1cnc2ccccc2n1 ; O=C1CCC(N2C(=O)c3ccc(F)cc3C2=O)C(=O)N1 ; Cc1cn(CCCCCCNc2ccc3c(c2)C(=O)N(C2CCC(=O)NC2=O)C3=O)nc1-c1cnc2ccccc2n1]

GS-546 was synthesized following the similar procedures as described for GS-521 (4 mg, yield over 3 steps: 9%) as yellow solid. MS (ESI) m/z=566.3 [M+H]⁺.

### Example 505. 5-((6-(4-(3-(Difluoromethyl)quinoxalin-2-yl)-1H-pyrazol-1-yl)hexyl)amino)-2-(2,6-dioxopiperidin-3-yl)isoindoline-1,3-dione (GS-547)

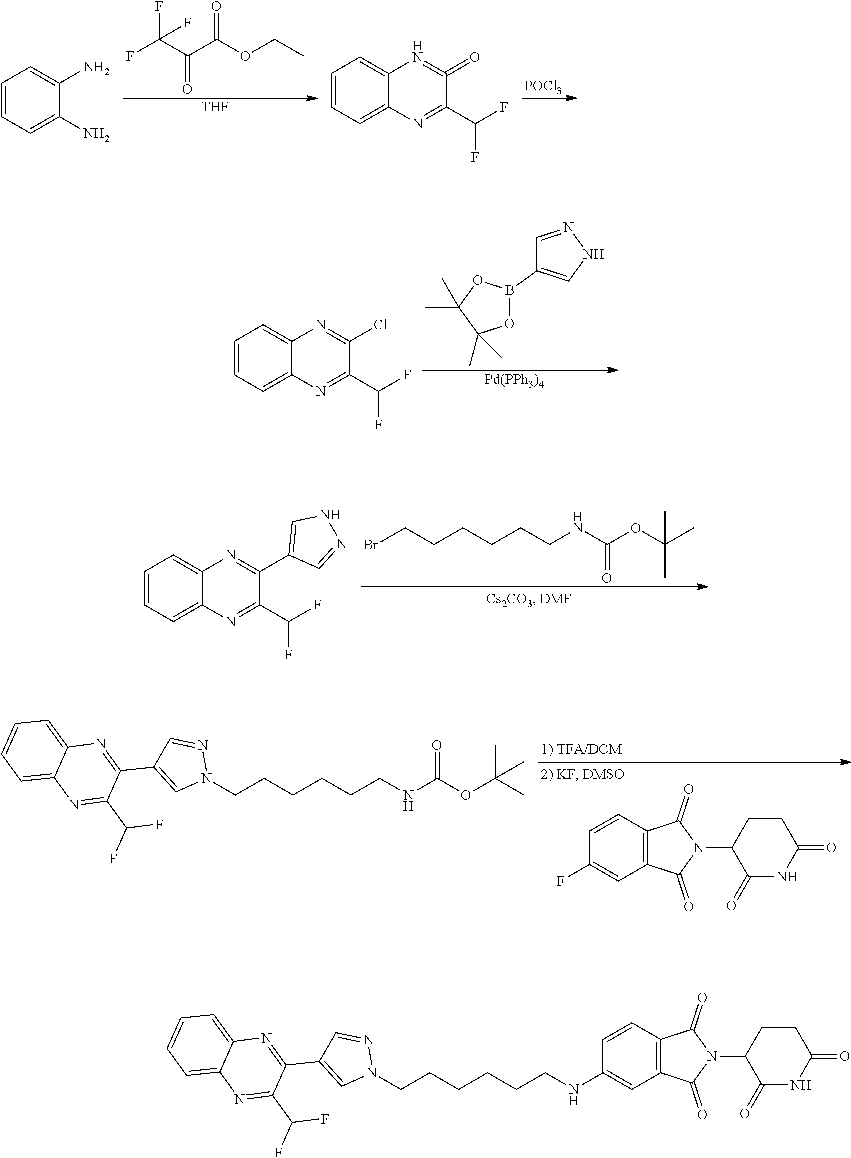

Step 1. Synthesis of 3(difluoromethyl)quinoxalin-2(1H)-one

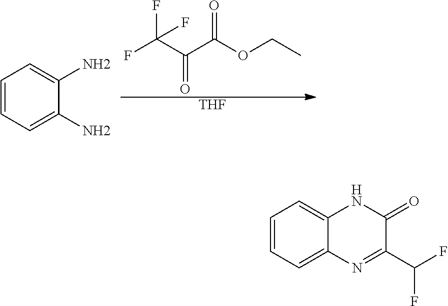

To a solution of benzene-1,2-diamine (5 g, 46.24 mmol) in THF (100 mL) was added ethyl 3,3,3-trifluoro-2-oxo-propanoate (8.65 g, 50.86 mmol) slowly at rt. The reaction mixture was heated at 50° C. for 24 h, before it was cooled to rt. Pd/C (1.5 g, 12.35 mmol) was added. The mixture was heated at 50° C. for another 24 h under H₂. After the reaction was cooled to rt, the mixture was filtered. To the filtrate was added NaOH solution (25%, 25 mL). The resulting mixture was heated at 45° C. for 5 h, before aq. HCl solution (3 M, 80 mL) was added. The organic layer was separated and concentrated. To the resulting residue was added methanol (10 mL). The mixture was heated at 50° C. for another 1 h, before it was cooled to 0° C. The suspension was filtered to provide the title compound (5.5 g, yield: 56%) as off white solid. MS (ESI) m/z=197.1 [M+H]⁺.

Step 2. Synthesis of 2-chloro-3-(difluoromethyl)quinoxaline

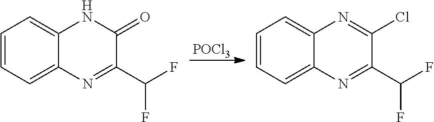

A solution of 3-(difluoromethyl)quinoxalin-2(1H)-one (550 mg, 2.58 mmol) in POCl₃ (6 mL) was heated at 100° C. for 5 h. The mixture was poured to water (60 mL) and extracted with EtOAc (60 mL×2). The combined organic layers were washed with brine (100 mL), dried and concentrated to provide the title compound (560 mg, yield: 93%) as light-yellow solid. MS (ESI) m/z=215.0 [M+H]⁺.

Step 3 to 5. Synthesis of 5-((6-(4-(3-(difluoromethyl)quinoxalin-2-yl)-1H-pyrazol-1-yl)hexyl)amino)-2-(2,6-dioxopiperidin-3-yl)isoindoline-1,3-dione

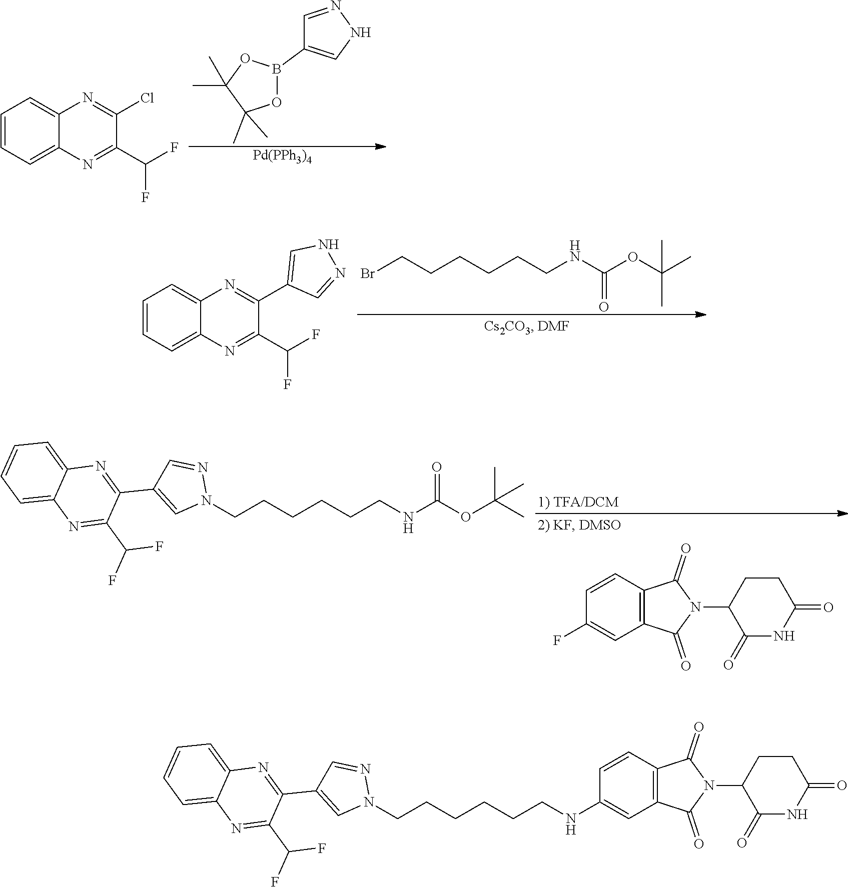

GS-547 was synthesized following the similar procedures as described for GS-521 (40 mg) as yellow solid. MS (ESI) m/z=602.4 [M+H]⁺.

### Example 506. 2-(2,6-Dioxopiperidin-3-yl)-5-((6-(4-(7-(1-methylpiperidin-4-yl)quinoxalin-2-yl)-1H-pyrazol-1-yl)hexyl)amino)isoindoline-1,3-dione (GS-548)

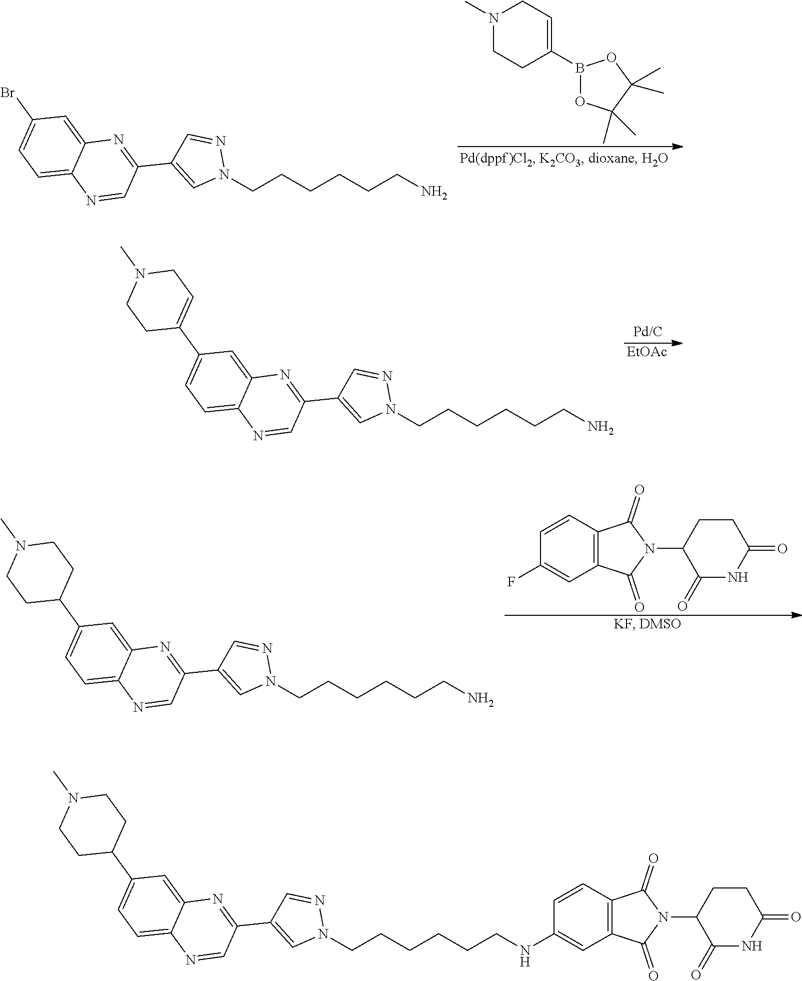

Step 1. Synthesis of 6-(4-(7-(1-methyl-1,2,3,6-tetrahydropyridin-4-yl)quinoxalin-2-yl)-1H-pyrazol-1-yl)hexan-1-amine

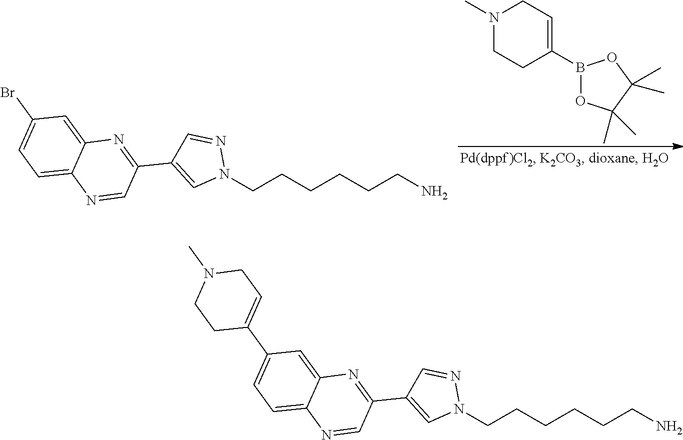

A mixture of 6-(4-(7-bromoquinoxalin-2-yl)-1H-pyrazol-1-yl)hexan-1-amine (see step 1 of GS-532 for synthesis, 50 mg, 133.59 μmol), 1-methyl-4-(4,4,5,5-tetramethyl-1,3,2-dioxaborolan-2-yl)-3,6-dihydro-2H-pyridine (44.71 mg, 200.39 μmol), Pd(dppf)Cl₂ (9.77 mg, 13.36 μmol), K₂CO₃ (36.87 mg, 267.18 μmol) and H₂O (0.4 mL) in dioxane (2 mL) was stirred at 90° C. for 1 h. The reaction was purified by reverse phase chromatography (water:MeOH=1:1) to provide the title compound (40 mg, yield: 77%) as yellow oil.

Step 2 to 4. Synthesis of 2-(2,6-dioxopiperidin-3-yl)-5-((6-(4-(7-(1-methylpiperidin-4-yl)quinoxalin-2-yl)-1H-pyrazol-1-yl)hexyl)amino)isoindoline-1,3-dione

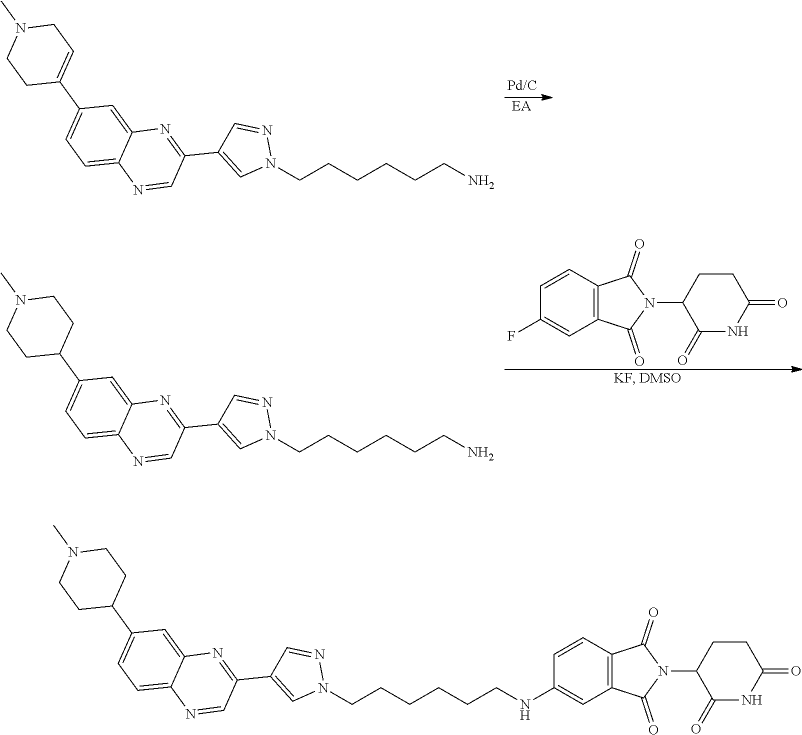

GS-548 was synthesized following the similar procedures as described for GS-541 (1.21 mg). MS (ESI) m/z=649.5 [M+H]⁺.

### Example 507. 5-((6-(3,5-Dimethyl-4-(quinolin-2-yl)-1H-pyrazol-1-yl)hexyl)amino)-2-(2,6-dioxopiperidin-3-yl)isoindoline-1,3-dione (GS-549)

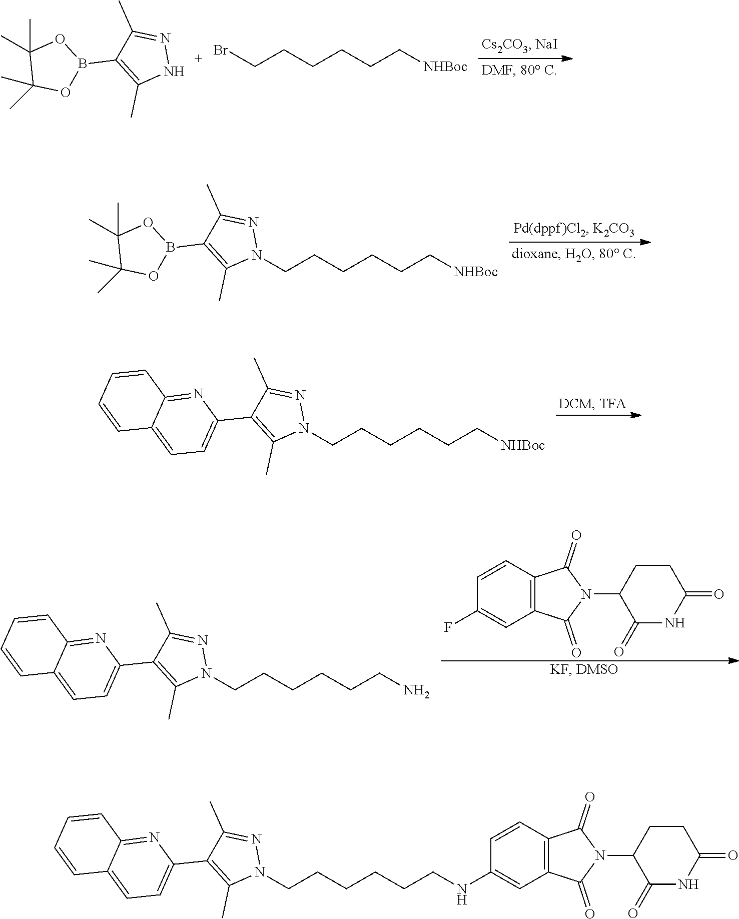

Step 1. Synthesis of tert-butyl (6-(3,5-dimethyl-4-(4,4,5,5-tetramethyl-1,3,2-dioxaborolan-2-yl)-1H-pyrazol-1-yl)hexyl)carbamate

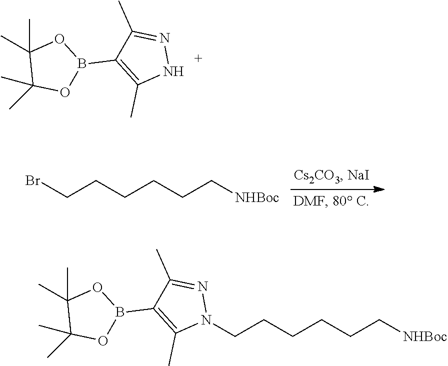 [SMILES: Cc1n[nH]c(C)c1B1OC(C)(C)C(C)(C)O1 ; CC(C)(C)OC(=O)NCCCCCCBr ; Cc1nn(CCCCCCNC(=O)OC(C)(C)C)c(C)c1B1OC(C)(C)C(C)(C)O1]

A solution of 3,5-dimethyl-4-(4,4,5,5-tetramethyl-1,3,2-dioxaborolan-2-yl)-1H-pyrazole (100 mg, 450.27 μmol), tert-butyl N-(6-bromohexyl)carbamate (151.40 mg, 540.32 μmol) and K₂CO₃ (124.46 mg, 900.53 μmol) in DMF (1 mL) was stirred at 70° C. under N₂ for 2 h. Then, the mixture was concentrated. The resulting residue was purified by reverse phase chromatography to provide the title compound (120 mg, yield: 63%). MS (ESI) m/z=422.4 [M+H]⁺.

Step 2. Synthesis of tert-butyl (6-(3,5-dimethyl-4-(quinolin-2-yl)-1H-pyrazol-1-yl)hexyl)carbamate

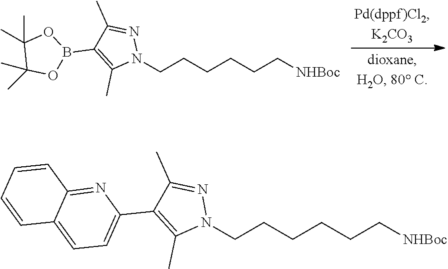 [SMILES: Cc1nn(CCCCCCNC(=O)OC(C)(C)C)c(C)c1B1OC(C)(C)C(C)(C)O1 ; Cc1nn(CCCCCCNC(=O)OC(C)(C)C)c(C)c1-c1ccc2ccccc2n1]

A solution of 2-bromoquinoline (45 mg, 216.29 μmol), tert-butyl (6-(3,5-dimethyl-4-(4,4,5,5-tetramethyl-1,3,2-dioxaborolan-2-yl)-1H-pyrazol-1-yl)hexyl)carbamate (118.48 mg, 281.18 μmol), Pd(dppf)Cl₂ (15.83 mg, 21.63 μmol) and K₂CO₃ (59.79 mg, 432.58 μmol) in dioxane (4 mL) and water (1 mL) was stirred at 80° C. under N₂ for 2 h. The mixture was concentrated, and the resulting residue was purified by silica gel chromatography to provide the title compound (60 mg, yield: 66%). MS (ESI) m/z=423.4 [M+H]⁺.

Step 3. Synthesis of 6-(3,5-dimethyl-4-(quinolin-2-yl)-1H-pyrazol-1-yl)hexan-1-amine

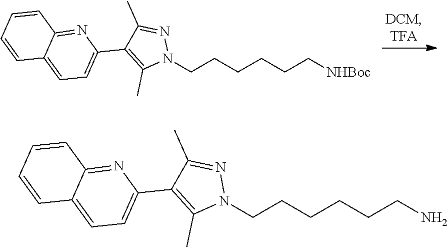 [SMILES: Cc1nn(CCCCCCNC(=O)OC(C)(C)C)c(C)c1-c1ccc2ccccc2n1 ; Cc1nn(CCCCCCN)c(C)c1-c1ccc2ccccc2n1]

A solution of tert-butyl (6-(3,5-dimethyl-4-(quinolin-2-yl)-1H-pyrazol-1-yl)hexyl)carbamate (60 mg, 141.99 μmol) in DCM (9 mL) and TFA (3 mL) was stirred at rt for 1 h. Then, the mixture was concentrated, and the resulting residue was purified by reverse phase chromatography to provide the title compound (40 mg, yield: 87%). MS (ESI) m/z=323.3 [M+H]⁺.

Step 4. Synthesis of 5-((6-(3,5-dimethyl-4-(quinolin-2-yl)-1H-pyrazol-1-yl)hexyl)amino)-2-(2,6-dioxopiperidin-3-yl)isoindoline-1,3-dione

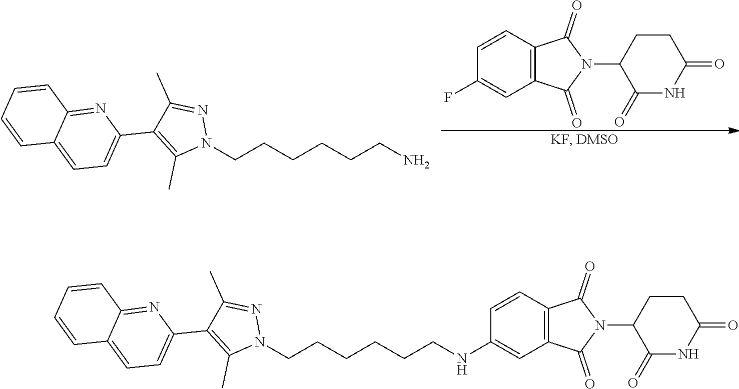

A solution of 6-(3,5-dimethyl-4-(quinolin-2-yl)-1H-pyrazol-1-yl)hexan-1-amine (40 mg, 124.05 μmol), 2-(2,6-dioxo-3-piperidyl)-5-fluoro-isoindoline-1,3-dione (51.40 mg, 186.08 μmol) and KF (14.41 mg, 248.10 μmol) in DMSO (2 mL) was irradiated at 130° C. in the microwave reactor for 15 min. Then, the mixture was purified by reverse phase chromatography to provide the title compound (2.01 mg, yield: 3%). MS (ESI) m/z=579.5 [M+H]⁺.

### Example 508. 2-(2,6-Dioxopiperidin-3-yl)-5-((6-(4-(quinoxalin-2-yl)-3-(trifluoromethyl)-1H-pyrazol-1-yl)hexyl)amino)isoindoline-1,3-dione (GS-550)

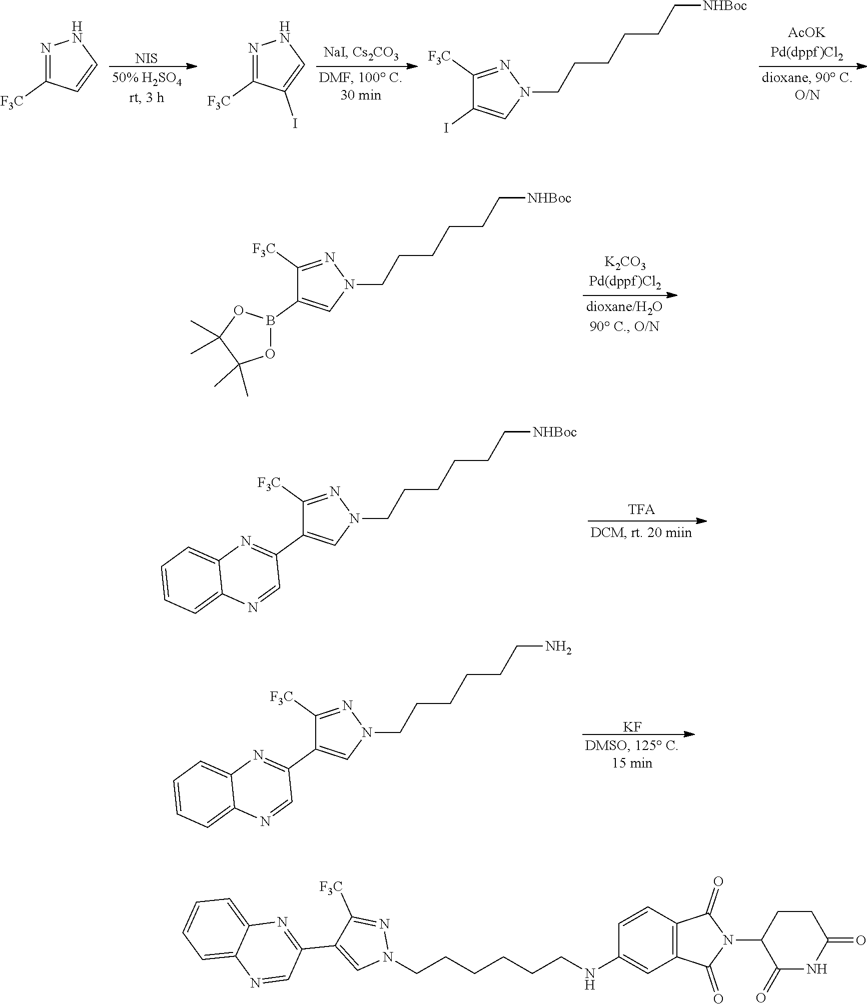

Step 1. Synthesis of 4-iodo-3-(trifluoromethyl)-1H-pyrazole

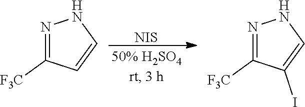

To a solution of 3-(trifluoromethyl)-1H-pyrazole (1.0 g, 7 mmol) in H₂SO₄ (15 mL) was added NIS (1.65 g, 7.3 mmol) at 0° C. After the reaction mixture was stirred at rt for 3 h, it was diluted with water. After filtration, the filter cake was dried to provide the title compound (1.4 g, yield: 73%) as white solid. MS (ESI) m/z=262.9 [M+H]⁺.

Step 2. Synthesis of tert-butyl (6-(4-iodo-3-(trifluoromethyl)-1H-pyrazol-1-yl)hexyl)carbamate

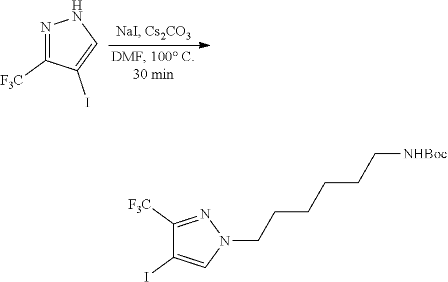 [SMILES: FC(F)(F)c1n[nH]cc1I ; CC(C)(C)OC(=O)NCCCCCCn1cc(I)c(C(F)(F)F)n1]

To a mixture of 4-iodo-3-(trifluoromethyl)-1H-pyrazole (500 mg, 1.9 mmol) and tert-butyl N-(7-bromoheptyl)carbamate (618 mg, 2.1 mmol) in DMF (10 mL) were added NaI (429 mg, 2.8 mmol) and CS₂CO₃ (1.24 g, 3.8 mmol). After the reaction was stirred at 100° C. for 1 h, it was quenched with water, and extracted with EtOAc. The organic phase was washed with water, dried over Na₂SO₄, filtered, and concentrated. The resulting residue was purified by reverse phase chromatography (0-70% a MeCN in H₂O) to provide the title compound (500 mg, yield: 57%) as bright oil. MS (ESI) m/z=462.1 [M+H]⁺.

Step 3. Synthesis of tert-butyl (6-(4-(4,4,5,5-tetramethyl-1,3,2-dioxaborolan-2-yl)-3-(trifluoromethyl)-1H-pyrazol-1-yl)hexyl)carbamate

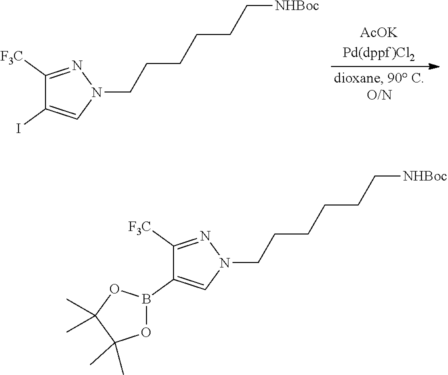 [SMILES: CC(C)(C)OC(=O)NCCCCCCn1cc(I)c(C(F)(F)F)n1 ; CC(C)(C)OC(=O)NCCCCCCn1cc(B2OC(C)(C)C(C)(C)O2)c(C(F)(F)F)n1]

To a mixture of tert-butyl (6-(4-iodo-3-(trifluoromethyl)-1H-pyrazol-1-yl)hexyl)carbamate (500 mg, 1.0 mmol) and Pin₂B (330 mg, 1.3 mmol) in DMSO (10 mL) were added Pd(dppf)Cl₂ (79 mg, 108 μmol) and AcOK (212 mg, 2.2 mmol). After the reaction mixture was stirred at 90° C. overnight, the reaction was quenched with water, and extracted with EtOAc. The organic phase was washed with water, dried over Na₂SO₄, filtered, and concentrated. The resulting residue was purified by reverse phase chromatography (0-70% MeCN in H₂O) to provide the title compound (100 mg, yield: 20%) as brown solid. MS (ESI) m/z=462.3 [M+H]⁺.

Step 4. Synthesis of tert-butyl (6-(4-(quinoxalin-2-yl)-3-(trifluoromethyl)-1H-pyrazol-1-yl)hexyl)carbamate

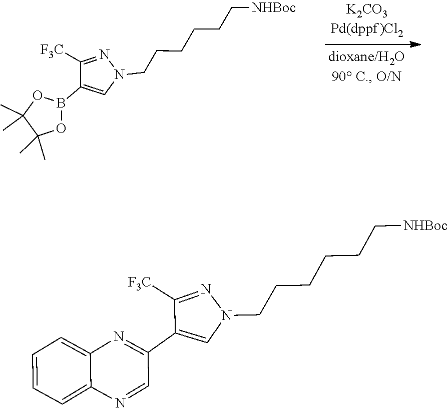 [SMILES: CC(C)(C)OC(=O)NCCCCCCn1cc(B2OC(C)(C)C(C)(C)O2)c(C(F)(F)F)n1 ; CC(C)(C)OC(=O)NCCCCCCn1cc(-c2cnc3ccccc3n2)c(C(F)(F)F)n1]

To a mixture of 2-chloroquinoxaline (30 mg, 182 μmol) and tert-butyl (6-(4-(4,4,5,5-tetramethyl-1,3,2-dioxaborolan-2-yl)-3-(trifluoromethyl)-1H-pyrazol-1-yl)hexyl)carbamate (126 mg, 273 μmol) in water (1 mL) and dioxane (5 mL) were added K₂CO₃ (50 mg, 364 μmol) and Pd(dppf)Cl₂ (13 mg, 18 μmol). After the reaction mixture was stirred at 90° C. overnight, the reaction was quenched with water, and extracted with EtOAc. The organic phase was washed with water, dried over Na₂SO₄, filtered, and concentrated. The resulting residue was purified by reverse phase chromatography (0-70% MeCN in H₂O) to provide the title compound (10 mg, yield: 12%) as brown solid. MS (ESI) m/z=464.2 [M+H]⁺.

Step 5. Synthesis of 6-(4-(quinoxalin-2-yl)-3-(trifluoromethyl)-1H-pyrazol-1-yl)hexan-1-amine

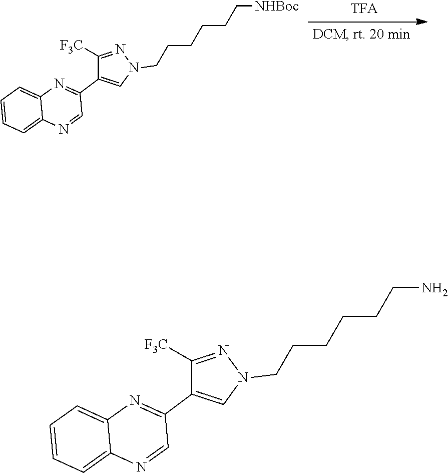 [SMILES: CC(C)(C)OC(=O)NCCCCCCn1cc(-c2cnc3ccccc3n2)c(C(F)(F)F)n1 ; NCCCCCCn1cc(-c2cnc3ccccc3n2)c(C(F)(F)F)n1]

To a solution of tert-butyl (6-(4-(quinoxalin-2-yl)-3-(trifluoromethyl)-1H-pyrazol-1-yl)hexyl)carbamate (10 mg, 21 μmol) in DCM (1 mL) was added TFA (0.5 mL). The reaction mixture was stirred at rt for 30 min, before it was concentrated. The resulting residue was purified by reverse phase chromatography (0-70% MeCN in H₂O) to provide the title compound (7 mg, yield: 89%) as yellow oil. MS (ESI) m/z=364.3 [M+H]⁺.

Step 6. Synthesis of 2-(2,6-dioxopiperidin-3-yl)-5-((6-(4-(quinoxalin-2-yl)-3-(trifluoromethyl)-1H-pyrazol-1-yl)hexyl)amino)isoindoline-1,3-dione

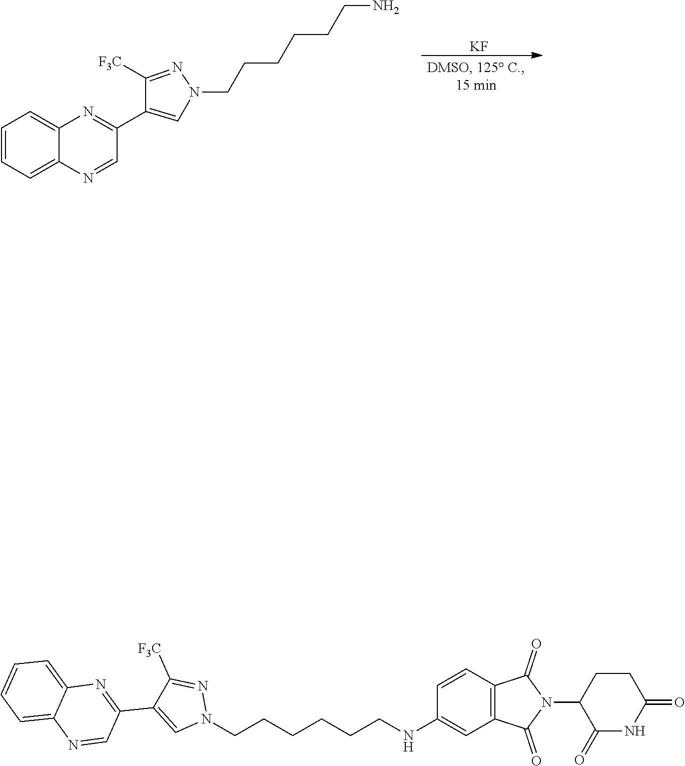

To a mixture of 6-(4-(quinoxalin-2-yl)-3-(trifluoromethyl)-1H-pyrazol-1-yl)hexan-1-amine (7 mg, 19 μmol) and 2-(2,6-dioxo-3-piperidyl)-5-fluoro-isoindoline-1,3-dione (6 mg, 21 μmol) in DMSO (0.3 mL) was added KF (1 mg, 19 μmol). After the reaction mixture was irradiated at 125° C. for 15 min in the microwave reactor, it was purified by chromatography (0-70% MeCN in H₂O) to provide the title compound (2.3 mg, yield: 19%) as a yellow solid. MS (ESI) m/z=620.5 [M+H]⁺.

### Example 509. 5-((5,5-Difluoro-6-(4-(quinoxalin-2-yl)-1H-pyrazol-1-yl)hexyl)amino)-2-(2,6-dioxopiperidin-3-yl)isoindoline-1,3-dione (GS-551)

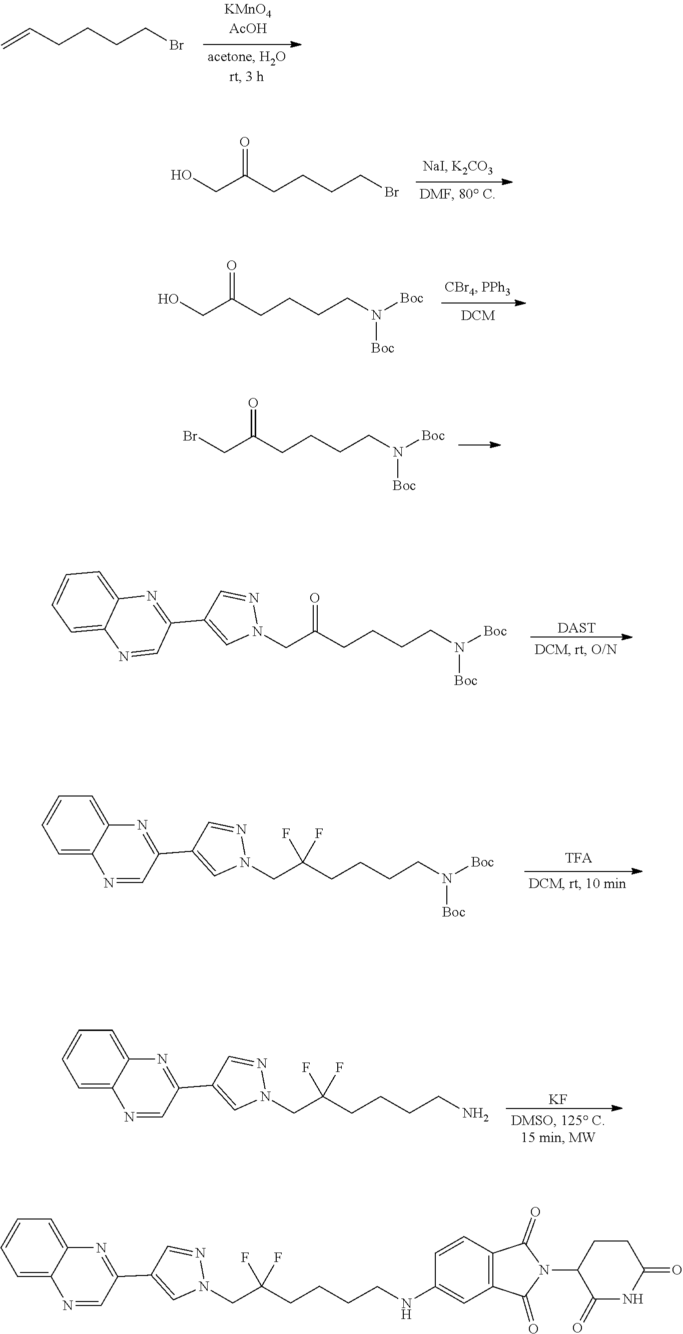

Step 1. Synthesis of 6-bromo-1-hydroxyhexan-2-one

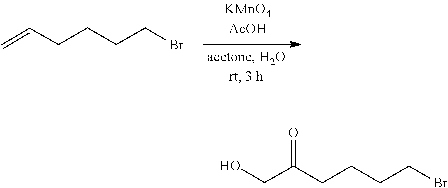

A solution of KMnO₄ (7.75 g, 49 mmol) in acetone (70 mL) and water (28 mL) was added to a mixture of 6-bromohex-1-ene (5.0 g, 30.6 mmol) in acetone (225 mL), water (75 mL) and AcOH (12 mL). The reaction mixture was stirred at rt for 3 h, before it was quenched with aqueous Na₂SO₃ solution, and concentrated to remove most of acetone. To the mixture was added DCM, and the mixture was filtered. The filtrate was extracted with DCM. The organic layer was washed with brine, filtered, and concentrated. The resulting residue was purified by reverse phase chromatography (0-70% MeCN in H₂O) to provide the title compound (3.4 g, yield: 57%) as bright oil. ¹H NMR (400 MHz, CDCl₃) δ 4.26 (s, 2H), 3.42 (dd, J=8.4, 4.4 Hz, 2H), 2.47 (t, J=7.1 Hz, 1H), 1.91-1.81 (m, 2H).

Step 2. Synthesis of tert-butyl N-tert-butoxycarbonyl-N-(6-hydroxy-5-oxo-hexyl)carbamate

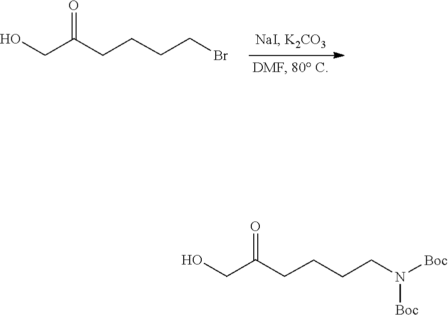 [SMILES: O=C(CO)CCCCBr ; CC(C)(C)OC(=O)N(CCCCC(=O)CO)C(=O)OC(C)(C)C]

To a mixture of 6-bromo-1-hydroxyhexan-2-one (2.0 g, 10.2 mmol) and tert-butyl N-tert-butoxycarbonylcarbamate (2.67 g, 12.3 mmol) in DMF (30 mL) were added NaI (2.3 g, 15.4 mmol) and K₂CO₃ (2.8 g, 20.5 mmol). The reaction mixture was stirred at 80° C. for 1 h, and then quenched with water. The mixture was extracted with EtOAc. The organic phase was washed with water, dried over Na₂SO₄, filtered, and concentrated. The resulting residue was purified by reverse phase chromatography (0-70% MeCN in H₂O) to provide the title compound (950 mg, yield: 28%) as bright oil. ¹H NMR (400 MHz, CDCl₃) δ 4.24 (d, J=4.8 Hz, 2H), 3.57 (t, J=7.0 Hz, 2H), 2.45 (t, J=7.1 Hz, 2H), 1.68-1.61 (m, 2H), 1.52-1.37 (m, 19H).

Step 3. Synthesis of tert-butyl N-(6-bromo-5-oxo-hexyl)-N-tert-butoxycarbonyl-carbamate

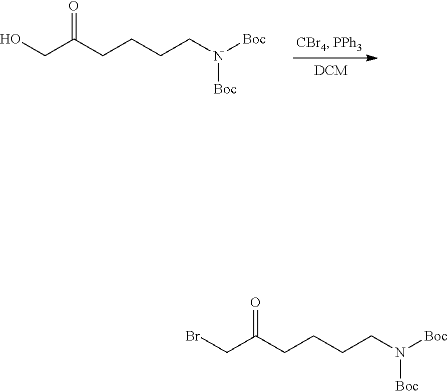 [SMILES: CC(C)(C)OC(=O)N(CCCCC(=O)CO)C(=O)OC(C)(C)C ; CC(C)(C)OC(=O)N(CCCCC(=O)CBr)C(=O)OC(C)(C)C]

To a mixture of tert-butyl N-tert-butoxycarbonyl-N-(6-hydroxy-5-oxo-hexyl)carbamate (950 mg, 2.8 mmol) in DCM (10 mL) were added CBr₄ (1.43 g, 4.3 mmol) and PPh₃ (1.1 g, 4.3 mmol). The reaction mixture was stirred at rt for 1 h, before it was quenched with water, and extracted with DCM. The organic phase was washed with water, dried over Na₂SO₄, filtered, and concentrated. The resulting residue was purified by reverse phase chromatography (0-70% MeCN in H₂O) to provide the title compound (400 mg, yield: 35%) as bright oil.

Step 4. Synthesis of tert-butyl N-tert-butoxycarbonyl-N-[5-oxo-6-(4-quinoxalin-2-ylpyrazol-1-yl)hexyl]carbamate

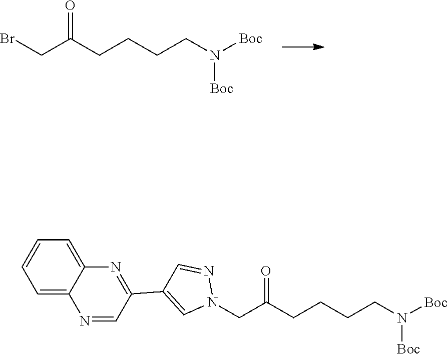 [SMILES: CC(C)(C)OC(=O)N(CCCCC(=O)CBr)C(=O)OC(C)(C)C ; CC(C)(C)OC(=O)N(CCCCC(=O)Cn1cc(-c2cnc3ccccc3n2)cn1)C(=O)OC(C)(C)C]

To a mixture of 2-(1H-pyrazol-4-yl)quinoxaline (90 mg, 458 μmol) and tert-butyl N-(6-bromo-5-oxo-hexyl)-N-tert-butoxycarbonyl-carbamate (217 mg, 550 μmol) in DMF (5 mL) were added NaI (103 mg, 688 μmol) and Cs₂CO₃ (299 mg, 917 μmol). The mixture was stirred at rt for 20 min, before it was quenched with water, and extracted with EtOAc. The organic phase was washed with water, dried over Na₂SO₄, filtered, and concentrated. The resulting residue was purified by reverse phase chromatography (0-70% MeCN in H₂O) to provide the title compound (130 mg, yield: 55%) as white solid. MS (ESI) m/z=510.3 [M+Na]⁺.

Step 5. Synthesis of tert-butyl N-tert-butoxycarbonyl-N-[5,5-difluoro-6-(4-quinoxalin-2-ylpyrazol-1-yl)hexyl]carbamate

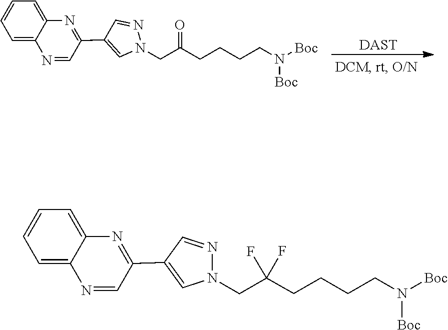

To a solution of tert-butyl N-tert-butoxycarbonyl-N-[5-oxo-6-(4-quinoxalin-2-ylpyrazol-1-yl)hexyl]carbamate (130 mg, 255 μmol) in DCM (3 mL) was added DAST (0.5 mL). The mixture was stirred at rit overnight, before it was quenched with aqueous NaHCO₃ solution. The resulting mixture was extracted with DCM. The organic phase was washed with water, dried over Na₂SO₄, filtered, and concentrated. The resulting residue was purified by reverse phase chromatography (0-70% MeCN in H₂O) to provide the title compound (110 mg, yield: 81%) as white solid. MS (ESI) m/z=532.3 [M+H]⁺.

Step 6. Synthesis of 5,5-difluoro-6-(4-(quinoxalin-2-yl)-1H-pyrazol-1-yl)hexan-1-amine

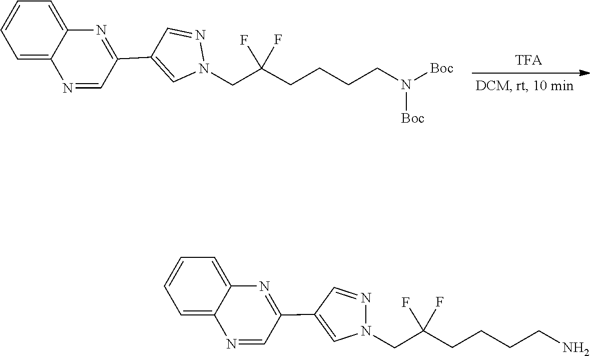 [SMILES: CC(C)(C)OC(=O)N(CCCCC(F)(F)Cn1cc(-c2cnc3ccccc3n2)cn1)C(=O)OC(C)(C)C ; NCCCCC(F)(F)Cn1cc(-c2cnc3ccccc3n2)cn1]

To a solution of tert-butyl N-tert-butoxycarbonyl-N-[5,5-difluoro-6-(4-quinoxalin-2-ylpyrazol-1-yl)hexyl]carbamate (110 mg, 207 μmol) in DCM (1 mL) was added TFA (0.5 mL). The reaction mixture was stirred at rt for 10 min before it was concentrated. The resulting residue was purified by reverse phase chromatography (0-70% MeCN in H₂O) to provide the title compound (60 mg, yield: 87%) as yellow oil. MS (ESI) m/z=332.2 [M+H]⁺.

Step 7. Synthesis of 5-((5,5-difluoro-6-(4-(quinoxalin-2-yl)-1H-pyrazol-1-yl)hexyl)amino)-2-(2,6-dioxopiperidin-3-yl)isoindoline-1,3-dione

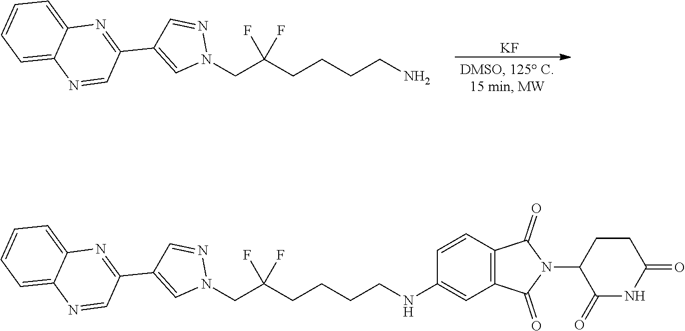 [SMILES: NCCCCC(F)(F)Cn1cc(-c2cnc3ccccc3n2)cn1 ; "KF" ; O=C1CCC(N2C(=O)c3ccc(NCCCCC(F)(F)Cn4cc(-c5cnc6ccccc6n5)cn4)cc3C2=O)C(=O)N1]

To a mixture of 5,5-difluoro-6-(4-(quinoxalin-2-yl)-1H-pyrazol-1-yl)hexan-1-amine (60 mg, 181 μmol) and 2-(2,6-dioxo-3-piperidyl)-5-fluoro-isoindoline-1,3-dione (55 mg, 199 μmol) in DMSO (3 mL) was added KF (10 mg, 181 μmol). The reaction mixture was irradiated at 125° C. for 15 min in the microwave reactor. The reaction was purified by reverse phase chromatography (0-70% MeCN in H₂O) to provide the title compound (87 mg, yield: 82%) as yellow solid. MS (ESI) m/z=588.4 [M+H]⁺.

### Example 510 and 511. 5-((6-(3-Cyclopropyl-4-(quinoxalin-2-yl)-1H-pyrazol-1-yl)hexyl)amino)-2-(2,6-dioxopiperidin-3-yl)isoindoline-1,3-dione (GS-552) and 5-((6-(5-cyclopropyl-4-(quinoxalin-2-yl)-1H-pyrazol-1-yl)hexyl)amino)-2-(2,6-dioxopiperidin-3-yl)isoindoline-1,3-dione (GS-553)

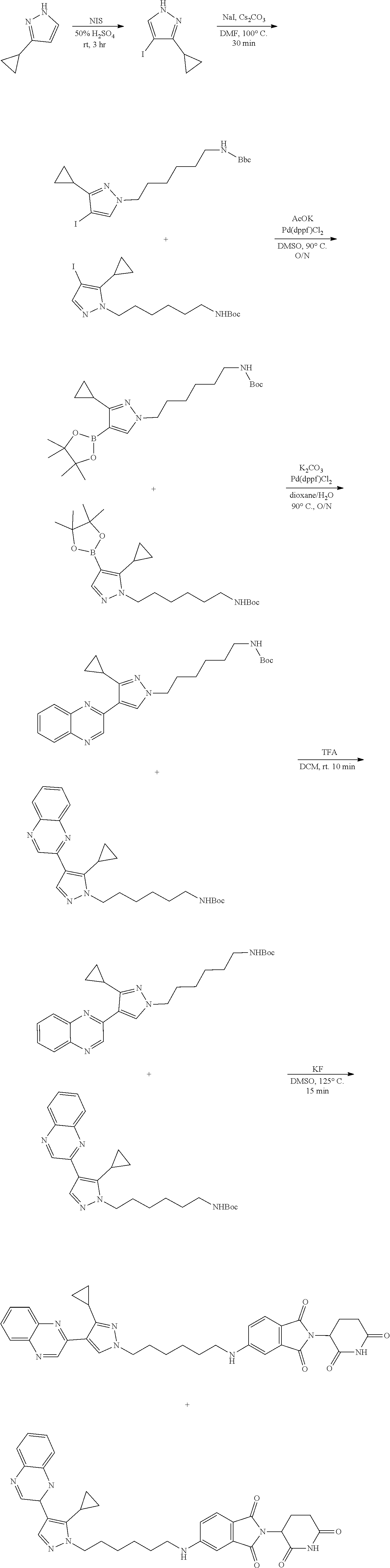

(GS-552, 22.1 mg, MS (ESI) m/z=592.4 [M+−H]; GS-553, 3.1 mg, yellow solid, MS (ESI) m/z=592.5 [M+H]⁺).

### Example 512. 2-(2,6-Dioxopiperidin-3-yl)-5-((6-(4-(quinoxalin-2-yl)-3,5-bis(trifluoromethyl)-1H-pyrazol-1-yl)hexyl)amino)isoindoline-1,3-dione (GS-554)

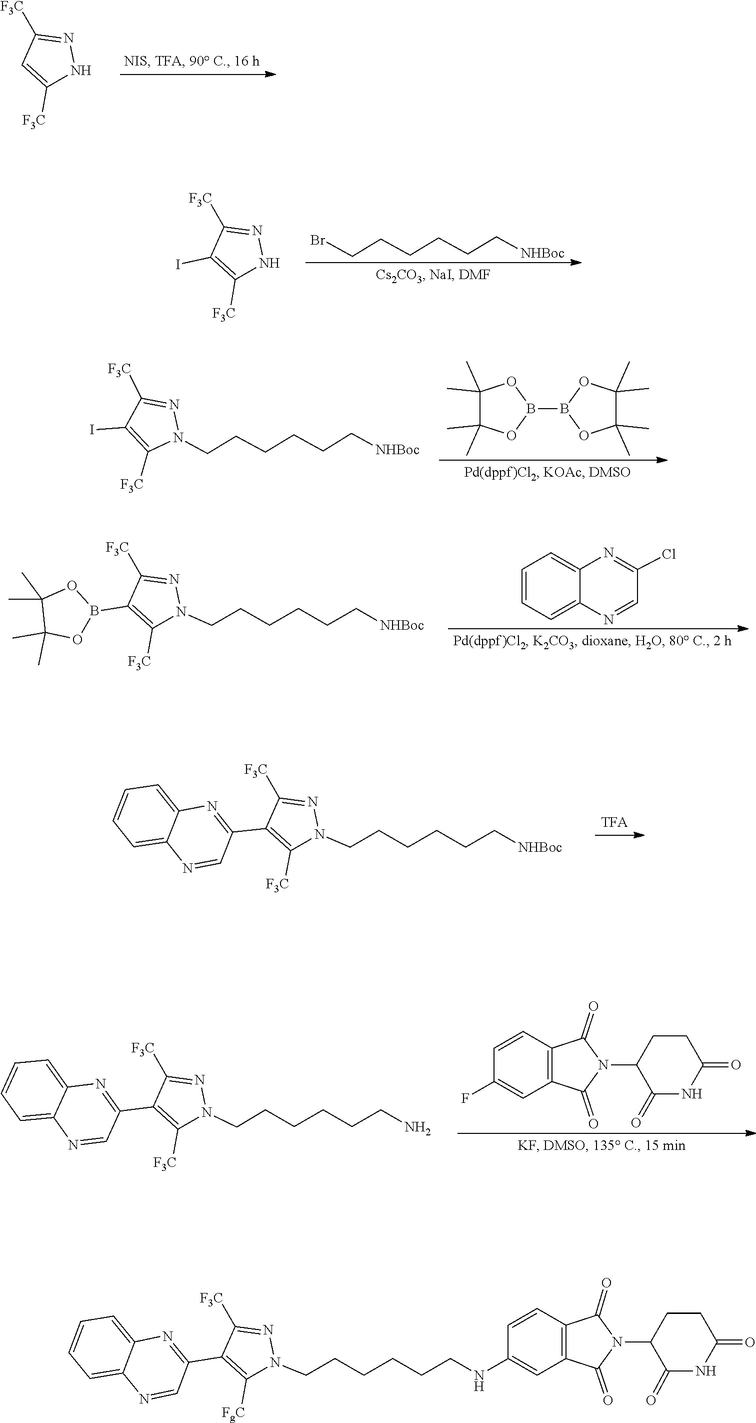

GS-554 was synthesized following the similar procedures as described for GS-550 (4 mg) as yellow solid. MS (ESI) m/z=688.4 [M+H]⁺.

### Example 513. 2-(2,6-Dioxopiperidin-3-yl)-5-((6-(4-(8-(trifluoromethyl)quinoxalin-2-yl)-1H-pyrazol-1-yl)hexyl)amino)isoindoline-1,3-dione (GS-555)

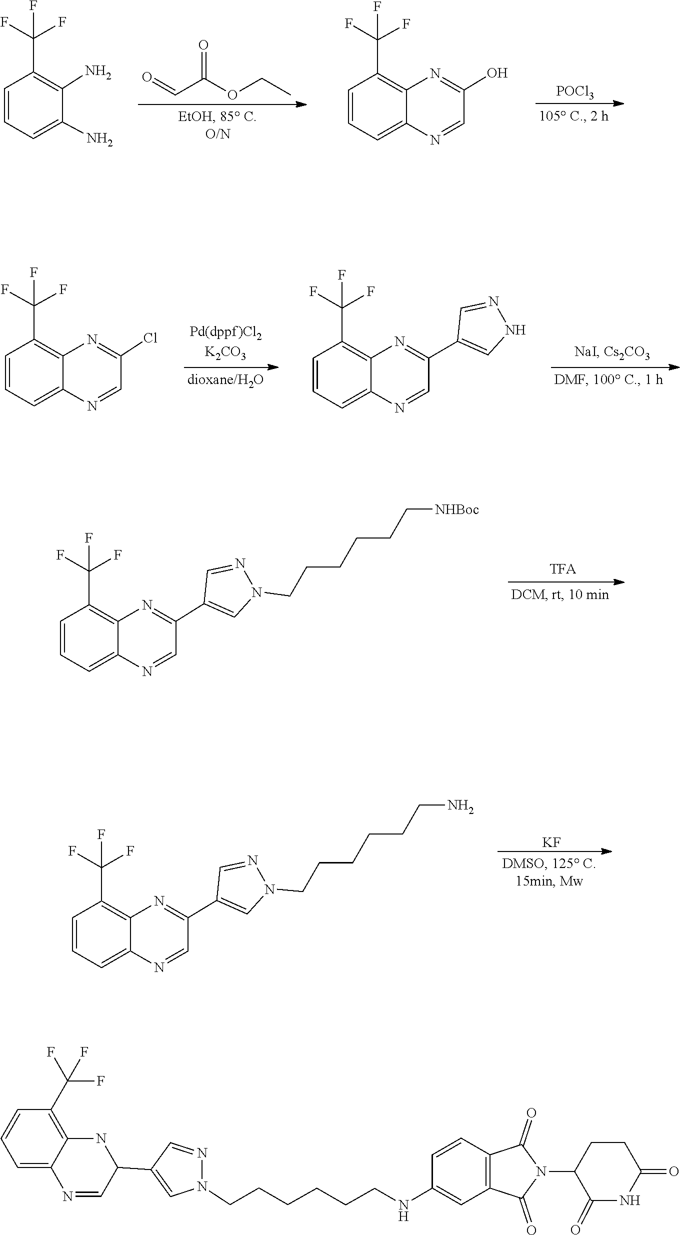

Step 1. Synthesis of 8-(trifluoromethyl)quinoxalin-2-ol

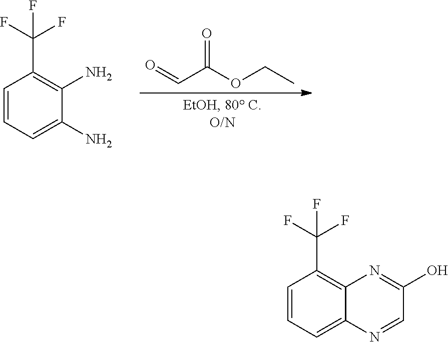

A mixture of 3-(trifluoromethyl)benzene-1,2-diamine (1.0 g, 5.6 mmol) and ethyl 2-oxoacetate (608 mg, 5.9 mmol) in EtOH (12 mL) was stirred at 85° C. overnight. The reaction was cooled to 0° C., and the precipitate was filtered to provide the title compound (270 mg, yield: 22%) as gray solid. MS (ESI) m/z=215.1 [M+H]⁺.

Step 2. Synthesis of 2-chloro-8-(trifluoromethyl)quinoxaline

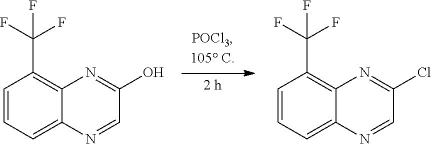 [SMILES: Oc1cnc2cccc(C(F)(F)F)c2n1 ; FC(F)(F)c1cccc2ncc(Cl)nc12]

A solution of 8-(trifluoromethyl)quinoxalin-2-ol (270 mg, 1.2 mmol) in POCl₃ (4 mL) was stirred at 105° C. for 2 h. After the mixture was concentrate, the resulting residue was purified by reverse phase chromatography (0-70% MeCN in H₂O) to provide the title compound (260 mg, yield: 88%) as white solid. MS (ESI) m/z=233.0 [M+H]⁺.

Step 3 to 6. Synthesis of 2-(2,6-dioxopiperidin-3-yl)-5-((6-(4-(8-(trifluoromethyl)quinoxalin-2-yl)-1H-pyrazol-1-yl)hexyl)amino)isoindoline-1,3-dione

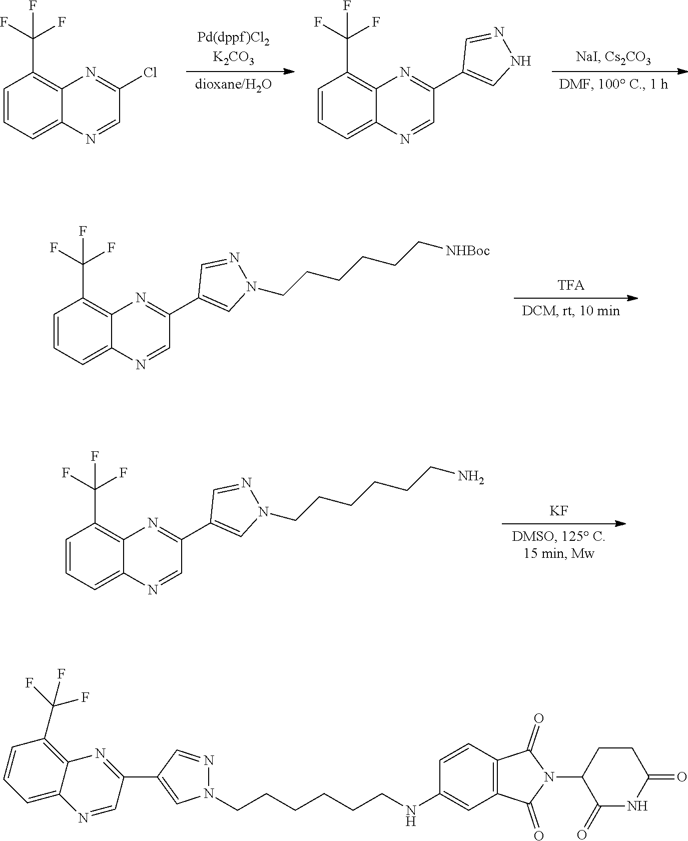 [SMILES: FC(F)(F)c1cccc2ncc(Cl)nc12 ; FC(F)(F)c1cccc2ncc(-c3cn[nH]c3)nc12 ; CC(C)(C)OC(=O)NCCCCCCn1cc(-c2cnc3cccc(C(F)(F)F)c3n2)cn1 ; NCCCCCCn1cc(-c2cnc3cccc(C(F)(F)F)c3n2)cn1 ; "KF" ; O=C1CCC(N2C(=O)c3ccc(NCCCCCCn4cc(-c5cnc6cccc(C(F)(F)F)c6n5)cn4)cc3C2=O)C(=O)N1]

GS-555 was synthesized following the similar procedures as described for GS-540 (38 mg) as yellow solid. MS (ESI) m/z=620.4 [M+H]⁺.

### Example 514. 3-(1,3-Dioxo-5-((3-(3-(4-(quinoxalin-2-yl)-1H-pyrazol-1-yl)propyl)cyclobutyl)amino)-2,3-dihydro-1H-inden-2-yl)piperidine-2,6-dione (GS-556)

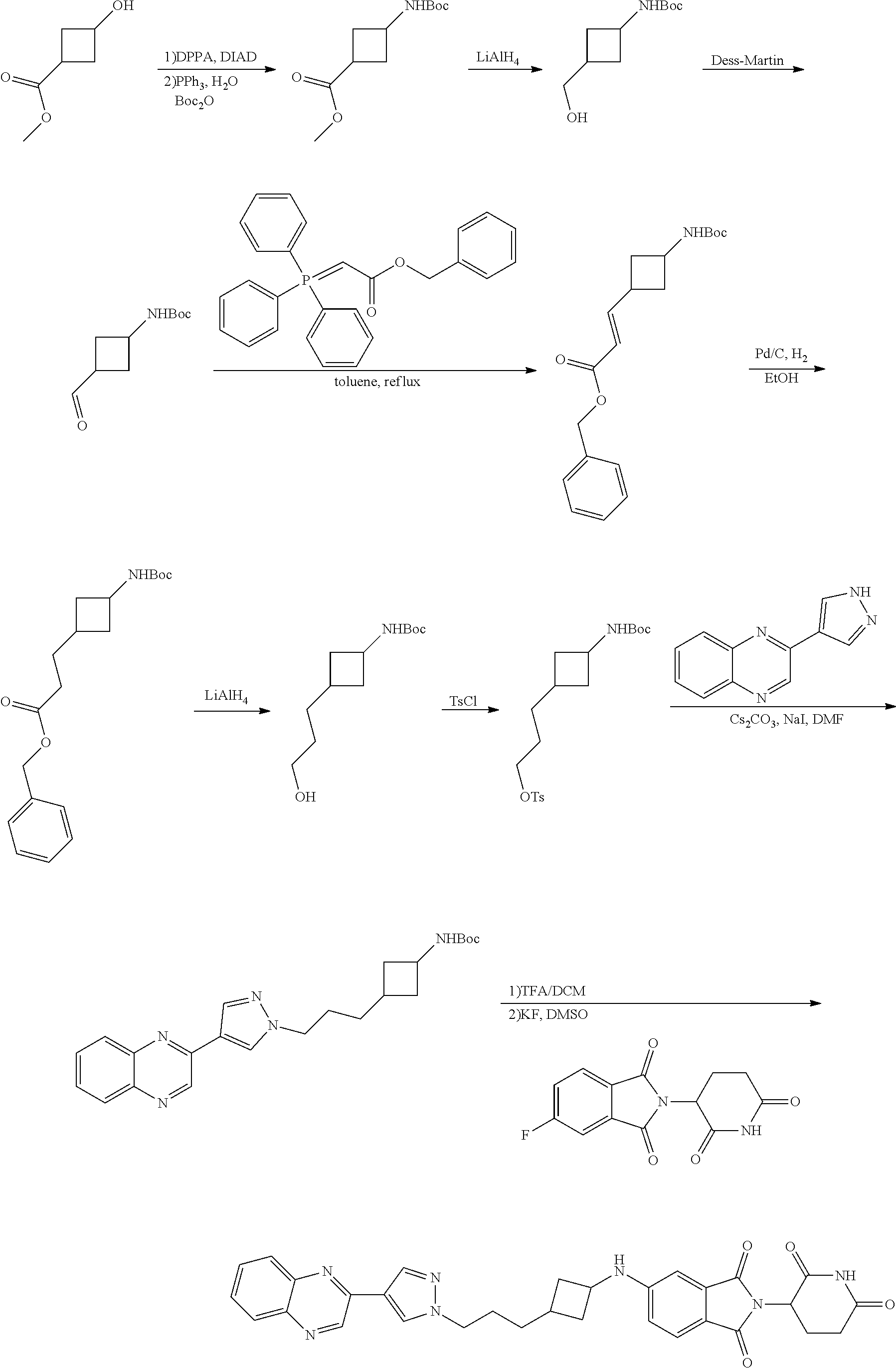

Step 1. Synthesis of methyl 3-(tert-butoxycarbonylamino)cyclobutanecarboxylate

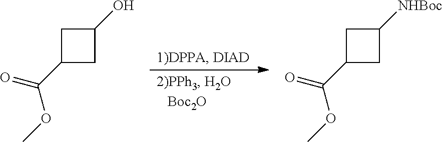

To a mixture of methyl 3-hydroxycyclobutanecarboxylate (2.0 g, 15.37 mmol) and triphenylphosphane (4.84 g, 18.44 mmol) in TH-F (20 mL) were added DIAD (3.73 g, 18.44 mmol) and DPPA (5.08 g, 18.44 mmol) at 0° C. under N₂. After the mixture was warmed to rt and stirred overnight, PPh₃ (13.5 g, 51.9 mmol), Boc₂O (13 g, 59.57 mmol) and water (10 mL) were added. The mixture was stirred at rt for another 2 h, and then stirred at 90° C. for 3 h. The mixture was cooled to rt, and extracted with EtOAc (100 mL). The organic phase was dried and concentrated. The resulting residue was purified by silica gel chromatography to provide the title compound (1.3 g, yield: 33%) as white solid. MS (ESI) m/z=174 [M+H−56]⁺.

Step 2. Synthesis of tert-butyl (3-(hydroxymethyl)cyclobutyl)carbamate

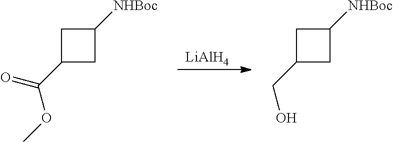 [SMILES: COC(=O)C1CC(NC(=O)OC(C)(C)C)C1 ; "LiAlH4" ; CC(C)(C)OC(=O)NC1CC(CO)C1]

To a solution of methyl 3-(tert-butoxycarbonylamino)cyclobutanecarboxylate (1.3 g, 5.10 mmol) in THF (20 mL) was added LiAlH₄ (260 mg, 6.84 mmol) at 0° C. The mixture was stirred at 0° C. for 2 h. The mixture was quenched with water, and filtered. The filtrate was concentrated to provide the title compound (900 mg, yield: 79%) as light-yellow oil.

Step 3. Synthesis of tert-butyl N-(3-formylcyclobutyl)carbamate

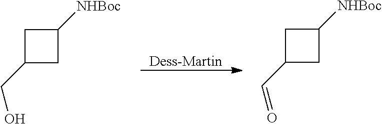 [SMILES: CC(C)(C)OC(=O)NC1CC(CO)C1 ; "Dess-Martin" ; CC(C)(C)OC(=O)NC1CC(C=O)C1]

To a solution of tert-butyl (3-(hydroxymethyl)cyclobutyl)carbamate (700 mg, 3.13 mmol) in DCM (20 mL) was added Dess-Martin Periodinane (2.10 g, 4.95 mmol) at 0° C. After the mixture was stirred at 0° C. for 5 h, it was filtered. The filtrate was concentrated. And the resulting residue was purified by silica gel chromatography to provide the title compound (500 mg, yield: 72%) as light-yellow oil.

Step 4. Synthesis of benzyl 3-(3-((tert-butoxycarbonyl)amino)cyclobutyl)acrylate

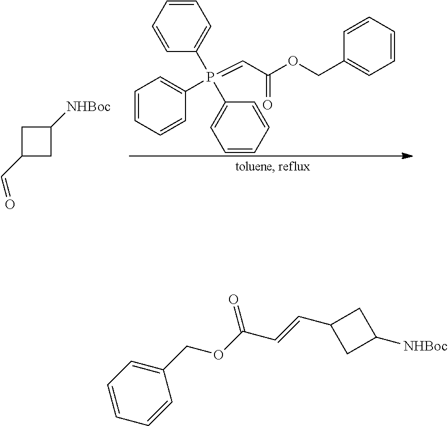 [SMILES: CC(C)(C)OC(=O)NC1CC(C=O)C1 ; O=C(C=P(c1ccccc1)(c1ccccc1)c1ccccc1)OCc1ccccc1 ; CC(C)(C)OC(=O)NC1CC(/C=C/C(=O)OCc2ccccc2)C1]

A mixture of tert-butyl N-(3-formylcyclobutyl)carbamate (500 mg, 2.26 mmol) and benzyl 2-(triphenyl-phosphanylidene)acetate (926.99 mg, 2.26 mmol) in toluene (15 mL) was heated at 90° C. overnight. The mixture was cooled to rt and concentrated. The resulting residue was purified by silica gel chromatography to provide the title compound (230 mg, yield: 25%) as light-yellow oil. MS (ESI) m/z=232.2 [M+H−100]⁺.

Step 5. Synthesis of benzyl 3-(3-((tert-butoxycarbonyl)amino)cyclobutyl)propanoate

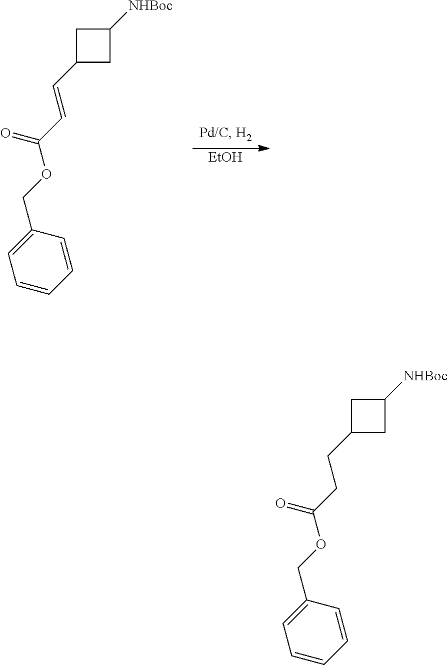 [SMILES: "EtOH" ; CC(C)(C)OC(=O)NC1CC(/C=C/C(=O)OCc2ccccc2)C1 ; CC(C)(C)OC(=O)NC1CC(CCC(=O)OCc2ccccc2)C1]

A mixture of benzyl 3-(3-((tert-butoxycarbonyl)amino)cyclobutyl)acrylate (230 mg, 569.09 μmol) and Pd/C (50 mg, 411.68 μmol) in ethanol (10 mL) was stirred under H₂ at rt overnight. The mixture was filtered, and the filtrate was concentrated to provide the title compound (210 mg, yield: 88%) as light-yellow oil. MS (ESI) m/z=234.2 [M+H]⁺.

Step 6. Synthesis of tert-butyl (3-(3-hydroxypropyl)cyclobutyl)carbamate

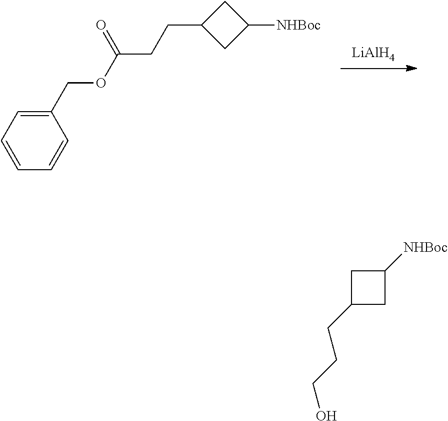 [SMILES: CC(C)(C)OC(=O)NC1CC(CCC(=O)OCc2ccccc2)C1 ; "LiAlH4" ; CC(C)(C)OC(=O)NC1CC(CCCO)C1]

To a solution of benzyl 3-(3-((tert-butoxycarbonyl)amino)cyclobutyl)propanoate (210 mg, 503.87 μmol) in THF (10 mL) was added LiAlH₄ (50 mg, 1.32 mmol) at 0° C. The mixture was stirred at 0° C. for 3 h. The mixture was quenched with water, and filtered. The filtrate was concentrated to provide the title compound (130 mg, yield: 90%) as light-yellow oil.

Step 7. Synthesis of 3-(3-((tert-butoxycarbonyl)amino)cyclobutyl)propyl 4-methylbenzenesulfonate

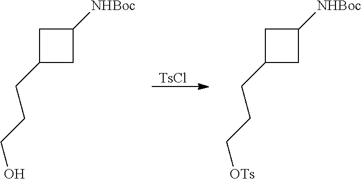

A mixture of tert-butyl (3-(3-hydroxypropyl)cyclobutyl)carbamate (130 mg, 453.52 μmol) and 4-methylbenzenesulfonyl chloride (130 mg, 681.89 μmol) in DCM (5 mL) were added DMAP (55 mg, 450.20 μmol) and DIEA (120 mg, 930.23 μmol). After the mixture was stirred at rt overnight, it was concentrated. The resulting residue was purified by silica gel chromatography to provide the title compound (100 mg, yield: 35%) as light-yellow oil. MS (ESI) m/z=284.2 [M+H−100]⁺.

Step 8. Synthesis of tert-butyl (3-(3-(4-(quinoxalin-2-yl)-1H-pyrazol-1-yl)propyl)cyclobutyl)carbamate

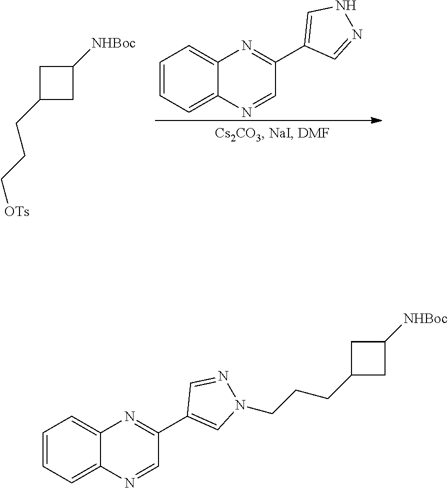 [SMILES: Cc1ccc(S(=O)(=O)OCCCC2CC(NC(=O)OC(C)(C)C)C2)cc1 ; c1ccc2nc(-c3cn[nH]c3)cnc2c1 ; CC(C)(C)OC(=O)NC1CC(CCCn2cc(-c3cnc4ccccc4n3)cn2)C1]

A mixture of 2-(1H-pyrazol-4-yl)quinoxaline (50 mg, 254.83 μmol), 3-(3-((tert-butoxycarbonyl)amino)cyclobutyl)propyl 4-methylbenzenesulfonate (100 mg, 156.45 μmol), NaI (30 mg, 200.14 μmol) and Cs₂CO₃ (120 mg, 368.32 μmol) in DMF (2 mL) was heated at 100° C. for 30 min. After the mixture was cooled to rt, it was purified by reverse phase chromatography to provide the title compound (60 mg, yield: 88%) as light-yellow solid. MS (ESI) m/z=408.4 [M+H]⁺.

Step 9. Synthesis of 3-(1,3-dioxo-5-((3-(3-(4-(quinoxalin-2-yl)-1H-pyrazol-1-yl)propyl)cyclobutyl)amino)-2,3-dihydro-1H-inden-2-yl)piperidine-2,6-dione

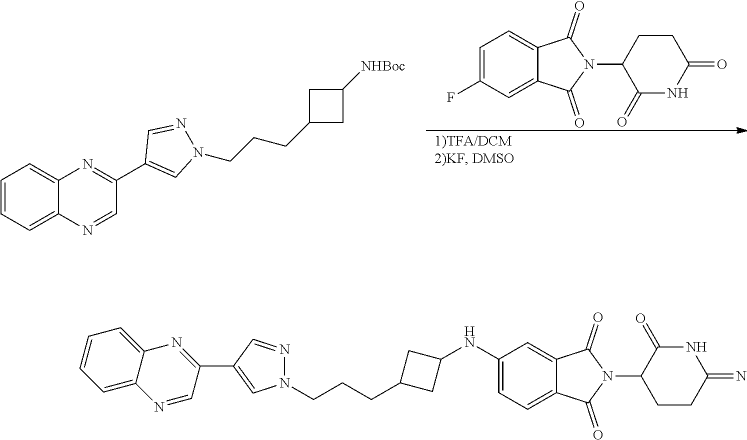 [SMILES: CC(C)(C)OC(=O)NC1CC(CCCn2cc(-c3cnc4ccccc4n3)cn2)C1 ; O=C1CCC(N2C(=O)c3ccc(F)cc3C2=O)C(=O)N1 ; N=C1CCC(N2C(=O)c3ccc(NC4CC(CCCn5cc(-c6cnc7ccccc7n6)cn5)C4)cc3C2=O)C(=O)N1]

To a solution of tert-butyl (3-(3-(4-(quinoxalin-2-yl)-1H-pyrazol-1-yl)propyl)cyclobutyl)carbamate (60 mg, 136.93 μmol) in DCM (3 mL) was added TFA (1 mL) at rt. The reaction mixture was stirred for 1 h, before it was concentrated. The resulting residue was dissolved in DCM (30 mL), washed with sat. Na₂CO₃ (10 mL) and brine (10 mL). The organic layer was dried and concentrated. To the resulting residue (80 mg, 290.66 μmol) were added KF (30 mg, 516.38 μmol) and DMSO (2 mL). The mixture was irradiated at 125° C. in the microwave reactor for 15 min. After the mixture was cooled to rt, it was purified by reverse phase chromatography to provide the title compound (16 mg, yield: 21%) as light-yellow solid. MS (ESI) m/z=564.4 [M+H]⁺.

### Example 515. 5-((6-(5-(Difluoromethyl)-4-(quinoxalin-2-yl)-1H-pyrazol-1-yl)hexyl)amino)-2-(2,6-dioxopiperidin-3-yl)isoindoline-1,3-dione (GS-557)

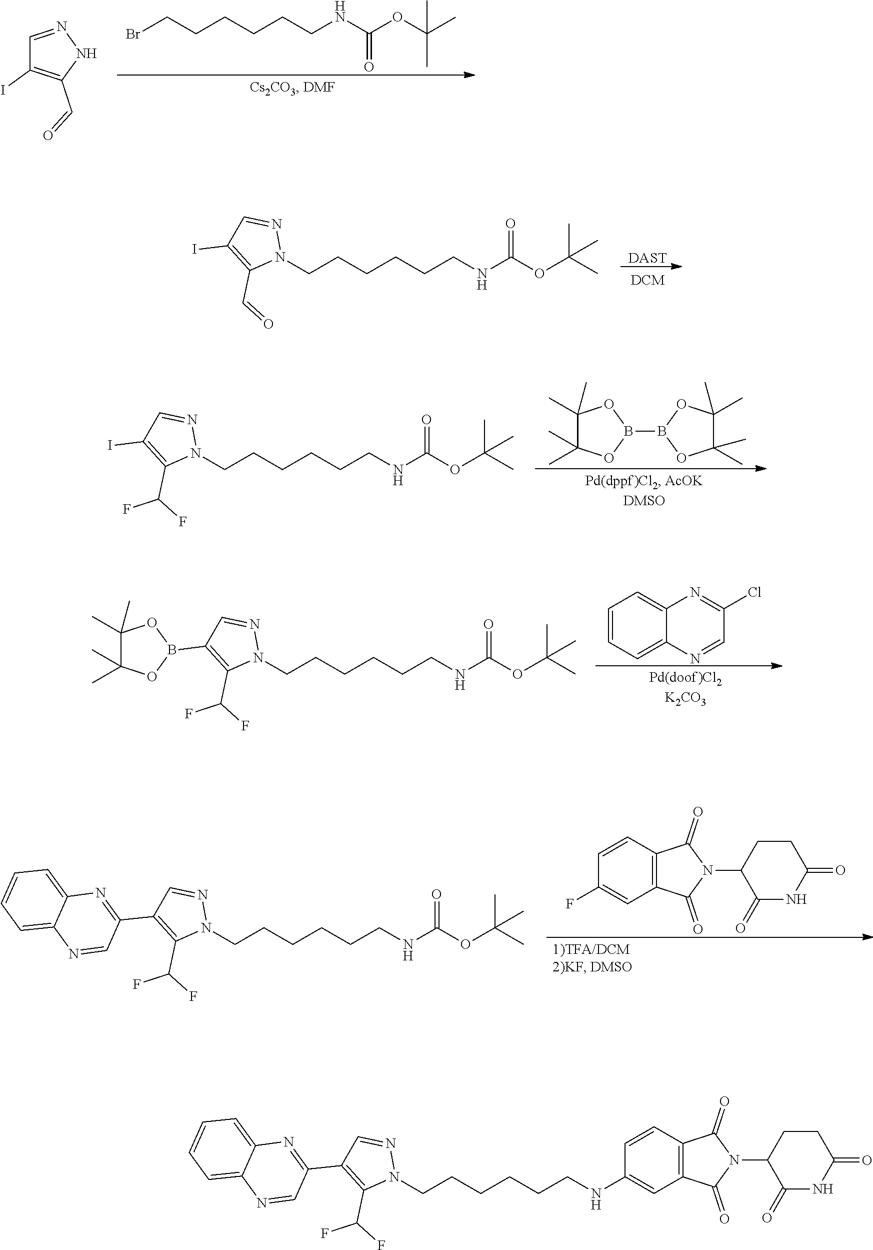

Step 1. Synthesis of tert-butyl (6-(5-formyl-4-iodo-1H-pyrazol-1-yl)hexyl)carbamate

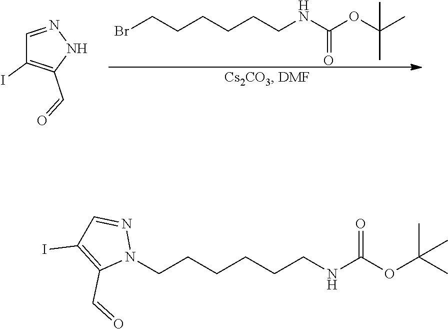 [SMILES: O=Cc1[nH]ncc1I ; CC(C)(C)OC(=O)NCCCCCCBr ; CC(C)(C)OC(=O)NCCCCCCn1ncc(I)c1C=O]

A mixture of 4-iodo-1H-pyrazole-3-carbaldehyde (see step 1 of GS-243 for synthesis, 300 mg, 1.08 mmol), tert-butyl N-(6-bromohexyl)carbamate (288.00 mg, 1.03 mmol), Cs₂CO₃ (720.00 mg, 2.21 mmol) and NaI (160 mg, 1.07 mmol) in DMF (3 mL) was heated at 100° C. for 0.5 h. After the mixture was cooled to rt, it was purified by reverse phase chromatography to provide the title compound (300 mg, yield: 63%) as brown oil. MS (ESI) m/z=322.1 [M+H−100]⁺.

Step 2. Synthesis of tert-butyl (6-(5-(difluoromethyl)-4-iodo-1H-pyrazol-1-yl)hexyl)carbamate

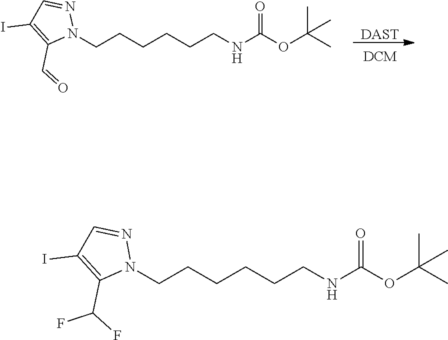

To a solution of tert-butyl (6-(5-formyl-4-iodo-1H-pyrazol-1-yl)hexyl)carbamate (300 mg, 676.52 μmol) in DCM (3 mL) was added DAST (1 mL) at 0° C. After the reaction mixture was stirred at rt overnight, it was poured into sat. NaHCO₃ solution (60 mL). The mixture was extracted with DCM (50 mL×2). The combined organic layers were washed with brine (50 mL), dried and concentrated. The resulting residue was purified by silica gel chromatography to provide the title compound (130 mg, yield: 39%) as light-yellow oil. MS (ESI) m/z=444.1 [M+H]⁺.

Step 3 to 5. Synthesis of 5-((6-(5-(difluoromethyl)-4-(quinoxalin-2-yl)-1H-pyrazol-1-yl)hexyl)amino)-2-(2,6-dioxopiperidin-3-yl)isoindoline-1,3-dione

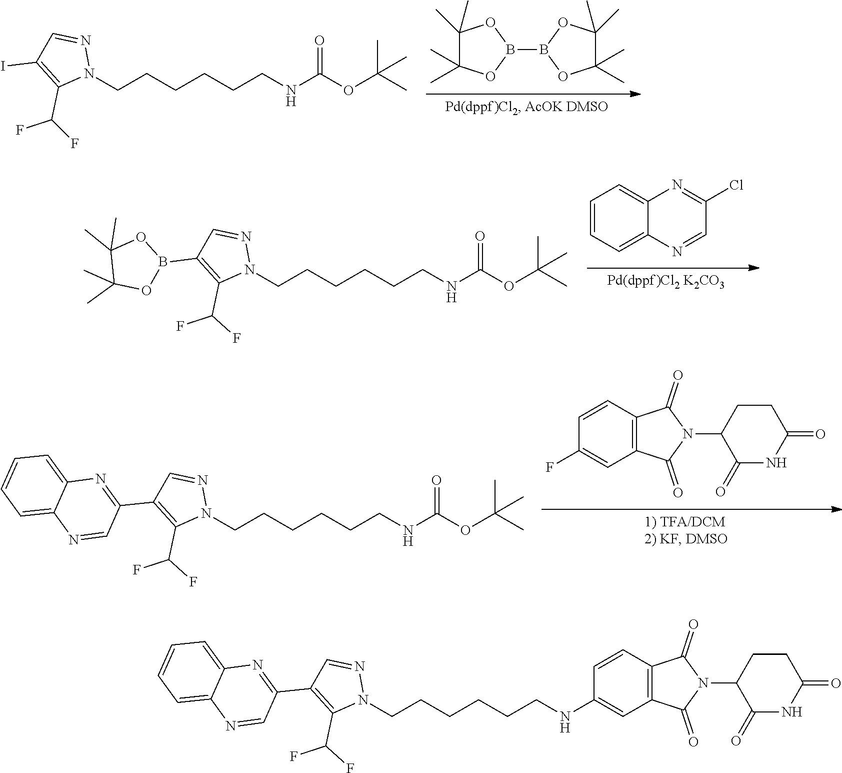

GS-557 was synthesized following the similar procedures as described for GS-550 (4.3 mg) as yellow solid. MS (ESI) m/z=602.6 [M+H]⁺.

### Example 516. 2-(2,6-Dioxopiperidin-3-yl)-5-((2-(3-((4-(quinoxalin-2-yl)-1H-pyrazol-1-yl)methyl)cyclobutyl)ethyl)amino)isoindoline-1,3-dione (GS-558)

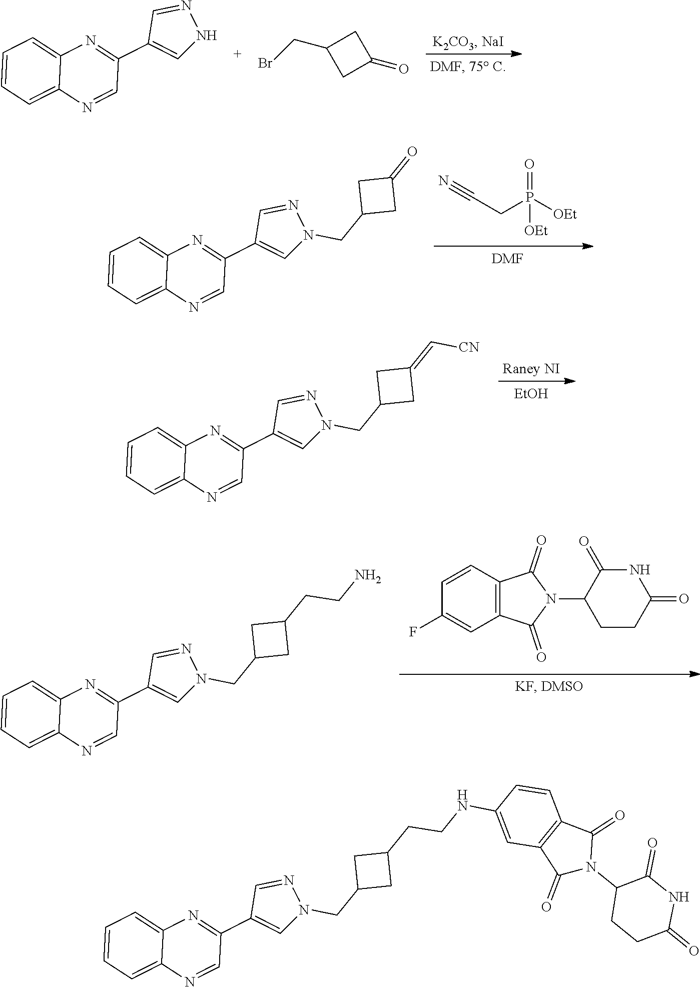

Step 1. Synthesis of 3-((4-(quinoxalin-2-yl)-1H-pyrazol-1-yl)methyl)cyclobutan-1-one

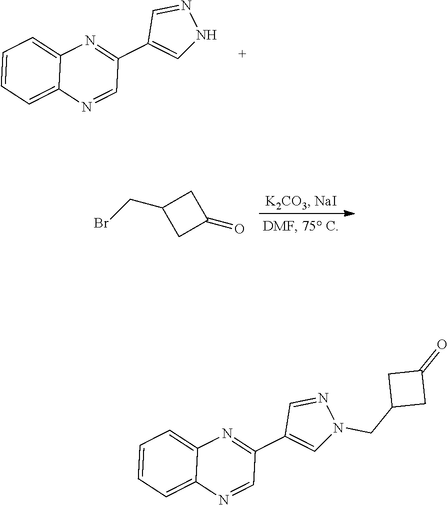

A mixture of 2-(1H-pyrazol-4-yl)quinoxaline (300 mg, 1.53 mmol), 3-(bromomethyl)cyclobutanone (324.02 mg, 1.99 mmol) and K₂CO₃ (422.64 mg, 3.06 mmol) in DMF (3 mL) was stirred at 70° C. under N₂ for 2 h. After the mixture was concentrated, the resulting residue was purified by reverse phase chromatography to provide the title compound (240 mg, yield: 56%). MS (ESI) m/z=279.2 [M+H]⁺.

Step 2. Synthesis of 2-(3-((4-(quinoxalin-2-yl)-1H-pyrazol-1-yl)methyl)cyclobutylidene)acetonitrile

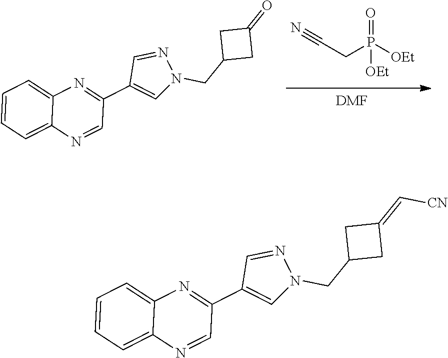

A solution of 2-diethoxyphosphorylacetonitrile (133.66 mg, 754.56 μmol) in DMF (2 mL) was stirred at 70° C. under N₂ for 30 min, before 3-((4-(Quinoxalin-2-yl)-1H-pyrazol-1-yl)methyl)cyclobutan-1-one (140 mg, 503.04 μmol) was added. The mixture was stirred at 70° C. for 3 h, before it was concentrated. The resulting residue was purified by reverse phase chromatography to provide the title compound (100 mg, yield: 66%). MS (ESI) m/z=302.2 [M+H]⁺.

Step 3. Synthesis of 2-(3-((4-(quinoxalin-2-yl)-1H-pyrazol-1-yl)methyl)cyclobutyl)ethan-1-amine

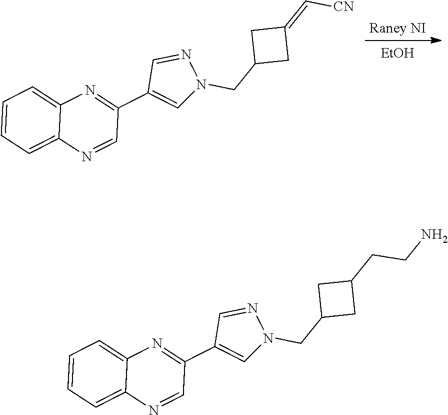 [SMILES: "EtOH" ; N#CC=C1CC(Cn2cc(-c3cnc4ccccc4n3)cn2)C1 ; NCCC1CC(Cn2cc(-c3cnc4ccccc4n3)cn2)C1]

A solution of 2-(3-((4-(quinoxalin-2-yl)-1H-pyrazol-1-yl)methyl)cyclobutylidene)acetonitrile (50 mg, 165.92 μmol) and Raney Ni (20 mg, 165.92 μmol) in ethanol (1 mL) was stirred at rt under H₂ overnight. The mixture was filtered. The filtrate was concentrated to provide the title compound (20 mg, yield: 39%). MS (ESI) m/z=308.2 [M+H]⁺.

Step 4. Synthesis of 2-(2,6-dioxopiperidin-3-yl)-5-((2-(3-((4-(quinoxalin-2-yl)-1H-pyrazol-1-yl)methyl)cyclobutyl)ethyl)amino)isoindoline-1,3-dione

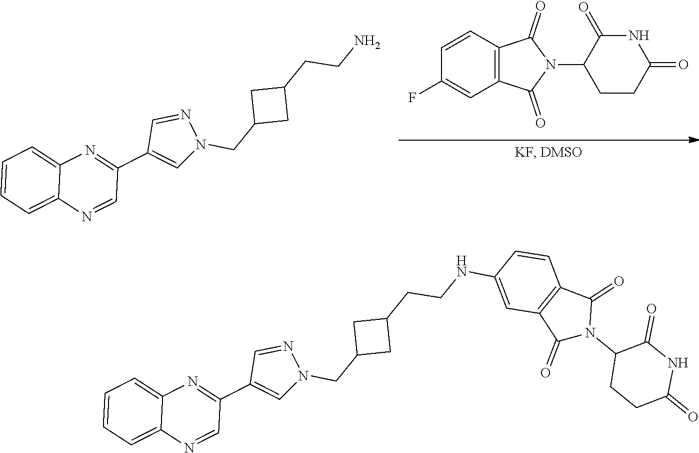 [SMILES: NCCC1CC(Cn2cc(-c3cnc4ccccc4n3)cn2)C1 ; O=C1CCC(N2C(=O)c3ccc(F)cc3C2=O)C(=O)N1 ; O=C1CCC(N2C(=O)c3ccc(NCCC4CC(Cn5cc(-c6cnc7ccccc7n6)cn5)C4)cc3C2=O)C(=O)N1]

A solution of 2-(3-((4-(quinoxalin-2-yl)-1H-pyrazol-1-yl)methyl)cyclobutyl)ethan-1-amine (20 mg, 65.06 μmol), 2-(2,6-dioxo-3-piperidyl)-5-fluoro-isoindoline-1,3-dione (26.96 mg, 97.60 μmol) and KF (7.56 mg, 130.13 μmol) in DMSO (1 mL) was irradiated at 130° C. in the microwave reactor for 15 min. Then, the mixture was purified by reverse phase chromatography to provide the title compound (2.77 mg, yield: 8%). MS (ESI) m/z=564.4 [M+H]⁺.

### Example 517. 5-((6-(3,5-Dimethyl-4-(3-(trifluoromethyl)quinoxalin-2-yl)-1H-pyrazol-1-yl)hexyl)amino)-2-(2,6-dioxopiperidin-3-yl)isoindoline-1,3-dione (GS-559)

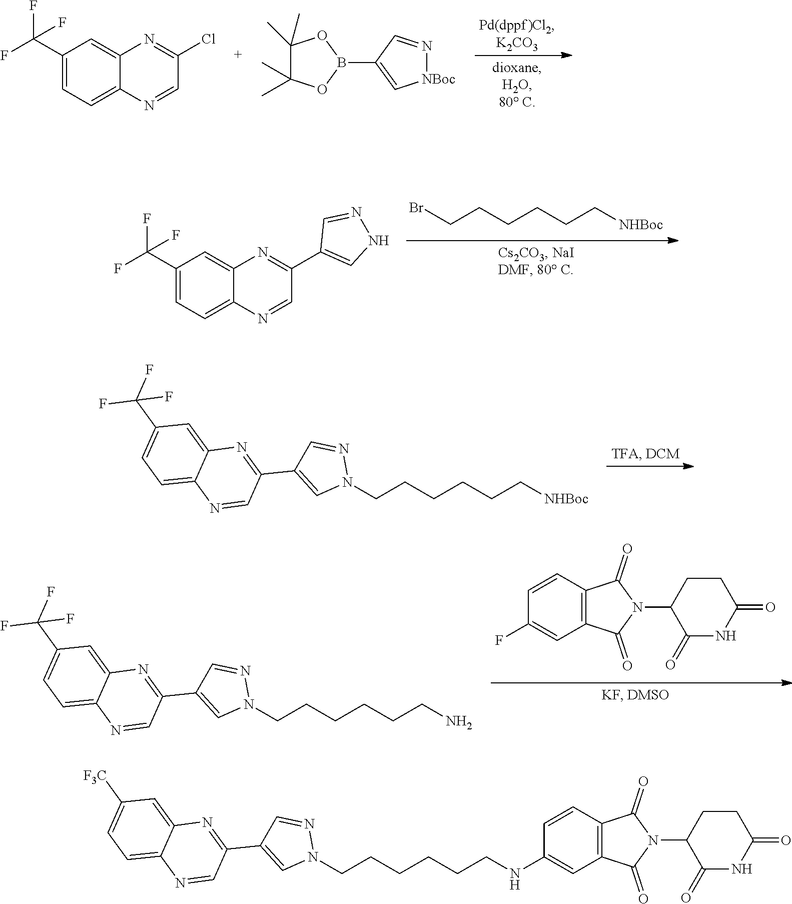

GS-559 was synthesized following the similar procedures as described for GS-540 (2.1 mg, yield: 6%). MS (ESI) m/z=620.4 [M+H]⁺.

### Example 518. 2-(2,6-Dioxopiperidin-3-yl)-5-((3-(3-(4-(7-(1-methylazetidin-3-yl)quinoxalin-2-yl)-1H-pyrazol-1-yl)cyclobutyl)propyl)amino)isoindoline-1,3-dione (GS-560)

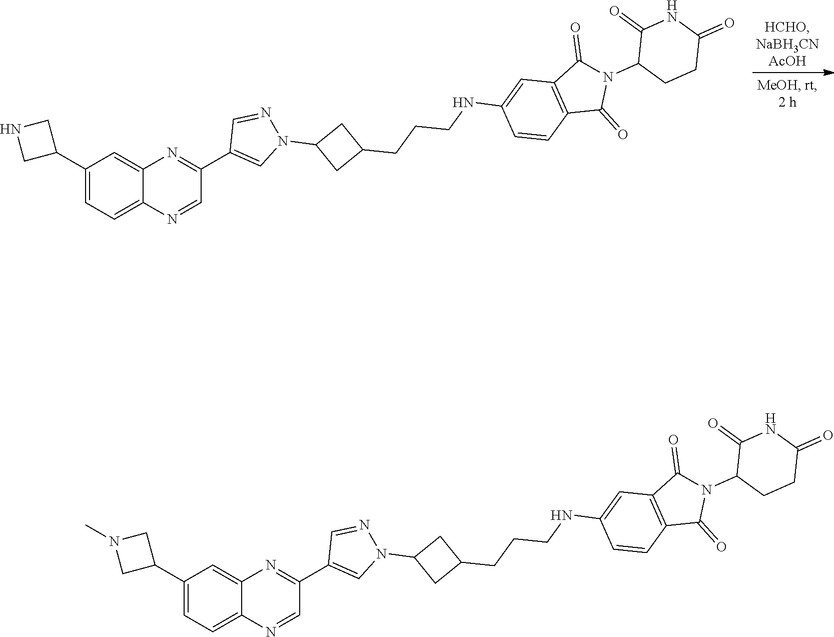 [SMILES: O=C1CCC(N2C(=O)c3ccc(NCCCC4CC(n5cc(-c6cnc7ccc(C8CNC8)cc7n6)cn5)C4)cc3C2=O)C(=O)N1 ; CN1CC(c2ccc3ncc(-c4cnn(C5CC(CCCNc6ccc7c(c6)C(=O)N(C6CCC(=O)NC6=O)C7=O)C5)c4)nc3c2)C1]

To a mixture of 5-((3-(3-(4-(7-(azetidin-3-yl)quinoxalin-2-yl)-1H-pyrazol-1-yl)cyclobutyl)propyl)amino)-2-(2,6-dioxopiperidin-3-yl)isoindoline-1,3-dione (GS-563, 14 mg, 23 μmol) in methanol (2 mL) were added formaldehyde (7 mg, 226 μmol), NaBH₃CN (4 mg, 68 μmol) and AcOH (0.1 mL). The reaction mixture was stirred at rt for 2 h, before it was concentrated. The resulting residue was purified by reverse phase chromatography (0-70% MeCN in H₂O) to provide the title compound (8.4 mg, yield: 50%) as brown solid. MS (ESI) m/z=633.6 [M+H]⁺.

### Example 519. 2-(2,6-Dioxopiperidin-3-yl)-5-((2-(3-(4-(quinoxalin-2-yl)-1H-pyrazol-1-yl)cyclobutyl)ethyl)amino)isoindoline-1,3-dione (GS-561)

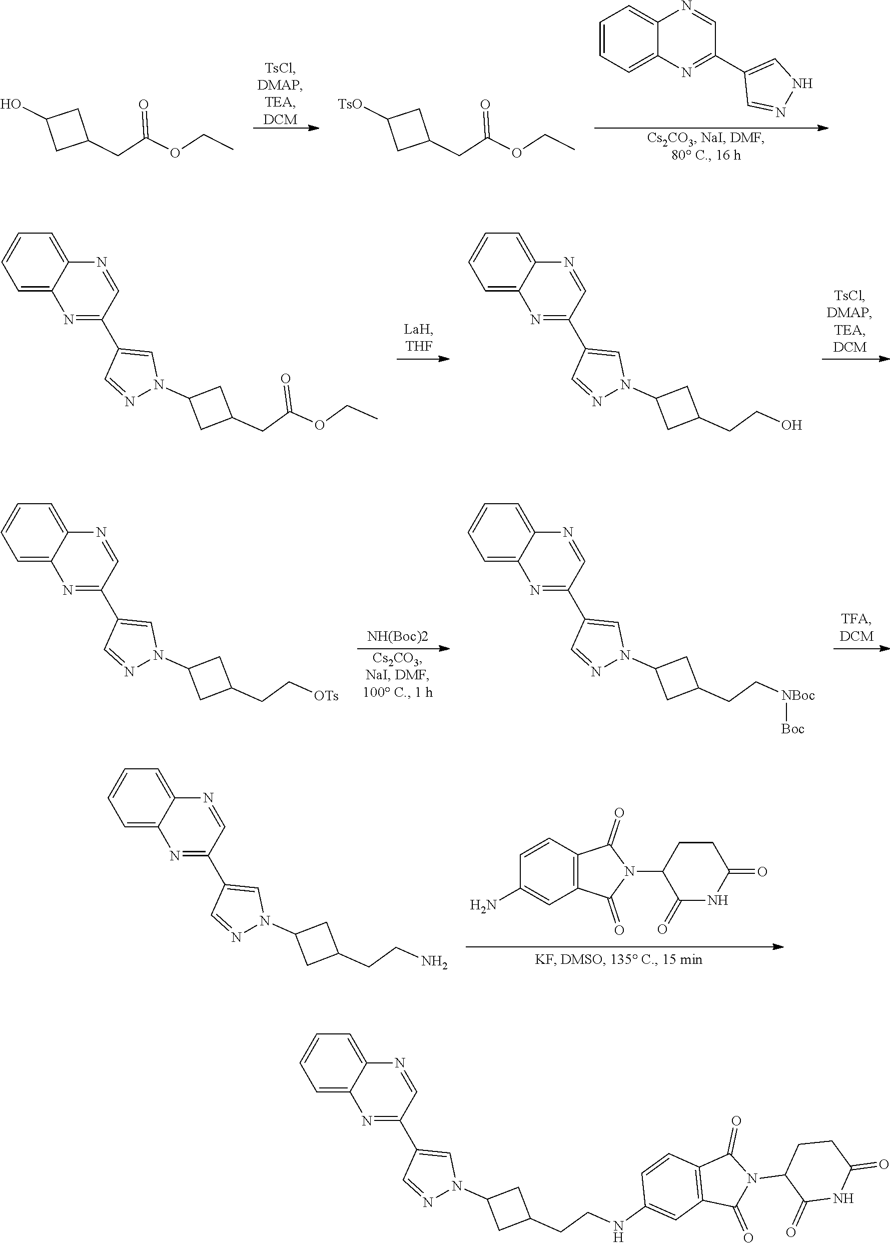

Step 1. Synthesis of ethyl 2-(3-(tosyloxy)cyclobutyl)acetate

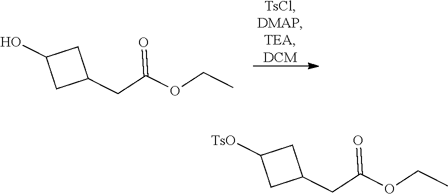 [SMILES: "TsCl,DMAP,TEA,DCM" ; CCOC(=O)CC1CC(O)C1 ; CCOC(=O)CC1CC(OS(=O)(=O)c2ccc(C)cc2)C1]

To a solution of ethyl 2-(3-hydroxycyclobutyl)acetate (300 mg, 1.9 mmol) in DCM (10 ml) were added TsCl (541 mg, 2.85 mmol), DMAP (23 mg, 0.19 mmol) and TEA (575 mg, 5.7 mmol) at 0° C. The mixture was stirred at rt for 16 h, and concentrated. The resulting residue was purified by silica gel chromatography (petroleum ether:EtOAc=5:1) to provide the title compound (490 mg, yield: 83%) as colorless solid. MS (ESI) m/z=313.3 [M+H]⁺.

Step 2. Synthesis of ethyl 2-(3-(4-(quinoxalin-2-yl)-1H-pyrazol-1-yl)cyclobutyl)acetate

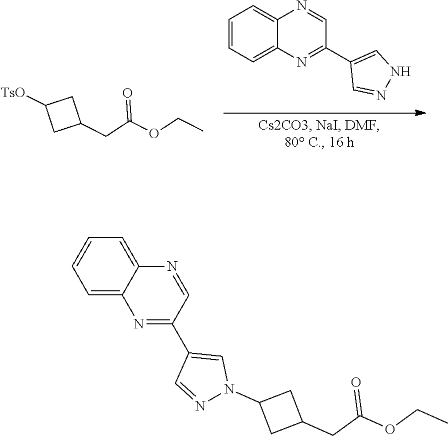

To a solution of ethyl 2-(3-(tosyloxy)cyclobutyl)acetate (490 mg, 1.57 mmol) in DMF (5 mL) were added 2-(1H-pyrazol-4-yl)quinoxaline (307 mg, 1.57 mmol), Cs₂CO₃ (1.02 g, 3.14 mmol) and NaI (235 mg, 1.57 mmol). The mixture was stirred at 80° C. for 16 h, and purified by reverse phase chromatography (MeOH:H₂O=2:1) to provide the title compound (340 mg, yield: 64%) as yellow oil. MS (ESI) m/z=337.3 [M+H]⁺.

Step 3. Synthesis of 2-(3-(4-(quinoxalin-2-yl)-1H-pyrazol-1-yl)cyclobutyl)ethan-1-ol

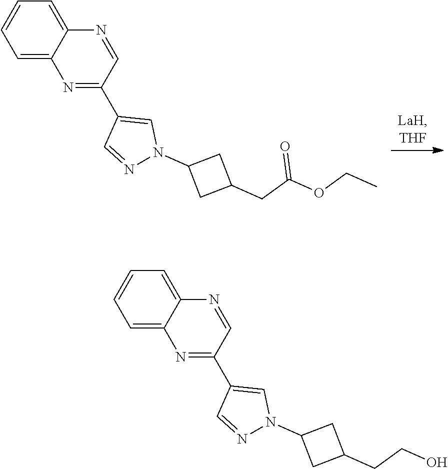 [SMILES: CCOC(=O)CC1CC(n2cc(-c3cnc4ccccc4n3)cn2)C1 ; OCCC1CC(n2cc(-c3cnc4ccccc4n3)cn2)C1]

To a solution of ethyl 2-(3-(4-(quinoxalin-2-yl)-1H-pyrazol-1-yl)cyclobutyl)acetate (340 mg, 1.01 mmol) in THF (10 mL) was added LiAlH₄ (77 mg, 2.02 mmol). The mixture was stirred at rt for 2 h. Then, 2 drops of water were added. The mixture was stirred for 30 min, and filtered. The filtrate was concentrated to provide the title compound (280 mg, yield: 94%) as yellow oil. MS (ESI) m/z=295.3 [M+H]⁺.

Step 4. Synthesis of 2-(3-(4-(quinoxalin-2-yl)-1H-pyrazol-1-yl)cyclobutyl)ethyl 4-methylbenzenesulfonate

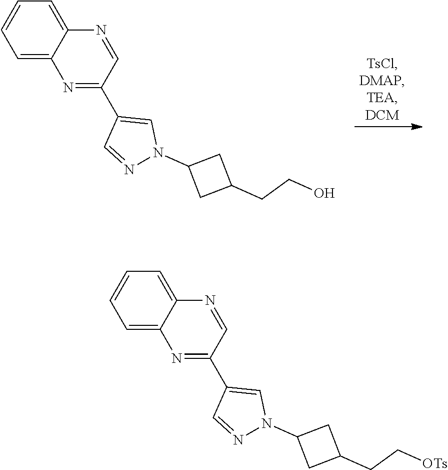 [SMILES: OCCC1CC(n2cc(-c3cnc4ccccc4n3)cn2)C1 ; Cc1ccc(S(=O)(=O)OCCC2CC(n3cc(-c4cnc5ccccc5n4)cn3)C2)cc1]

To a solution of 2-(3-(4-(quinoxalin-2-yl)-1H-pyrazol-1-yl)cyclobutyl)ethan-1-ol (280 mg, 0.95 mmol) in DCM (10 mL) were added TsCl (270 mg, 1.4 mmol), TEA (288 mg, 2.85 mmol) and DMAP (11 mg, 0.095 mmol). The mixture was stirred at rt for 16 h, and concentrated. The resulting residue was purified by silica gel chromatography (petroleum ether:EtOAc=2:1) to provide the title compound (300 mg, yield: 70%) as colorless oil. MS (ESI) m/z=449.3 [M+H]⁺.

Step 5. Synthesis of tert-butyl N-tert-butoxycarbonyl-N-[2-[3-(4-quinoxalin-2-ylpyrazol-1-yl)cyclobutyl]ethyl]carbamate

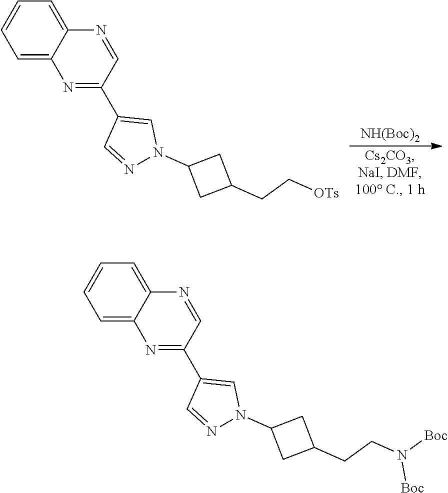 [SMILES: Cc1ccc(S(=O)(=O)OCCC2CC(n3cc(-c4cnc5ccccc5n4)cn3)C2)cc1 ; "Cs2CO3, NaI, DMF,100° C., 1 h" ; CC(C)(C)OC(=O)N(CCC1CC(n2cc(-c3cnc4ccccc4n3)cn2)C1)C(=O)OC(C)(C)C ; "NH(Boc)2"]

To a solution of 2-(3-(4-(quinoxalin-2-yl)-1H-pyrazol-1-yl)cyclobutyl)ethyl 4-methylbenzenesulfonate (200 mg, 0.44 mmol) in DMF (5 mL) were added di-tert-butyl iminodicarboxylate (143 mg, 0.66 mmol), Cs₂CO₃ (286 mg, 0.88 mmol) and NaI (66 mg, 0.44 mmol). The mixture was stirred at 90° C. for 2 h, before it was purified by silica gel chromatography (petroleum ether:EtOAc=2:1) to provide the title compound (180 mg, yield: 82%). MS (ESI) m/z=494.3 [M+H]⁺.

Step 6. Synthesis of 2-(3-(4-(quinoxalin-2-yl)-1H-pyrazol-1-yl)cyclobutyl)ethan-1-amine

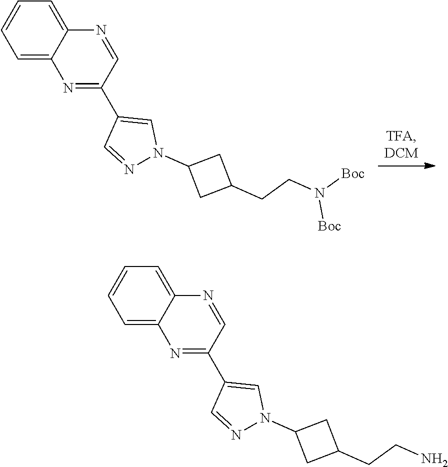 [SMILES: CC(C)(C)OC(=O)N(CCC1CC(n2cc(-c3cnc4ccccc4n3)cn2)C1)C(=O)OC(C)(C)C ; NCCC1CC(n2cc(-c3cnc4ccccc4n3)cn2)C1]

To a solution of tert-butyl N-tert-butoxycarbonyl-N-[2-[3-(4-quinoxalin-2-ylpyrazol-1-yl)cyclobutyl]ethyl]carbamate (180 mg, 0.36 mmol) in DCM (2 mL) was added TFA (2 mL). The mixture was stirred at rt for 30 min, before it was concentrated to provide the title compound (105 mg, yield: 98%). MS (ESI) m/z=294.2 [M+H]⁺.

Step 7. Synthesis of 2-(2,6-dioxopiperidin-3-yl)-5-((2-(3-(4-(quinoxalin-2-yl)-1H-pyrazol-1-yl)cyclobutyl)ethyl)amino)isoindoline-1,3-dione

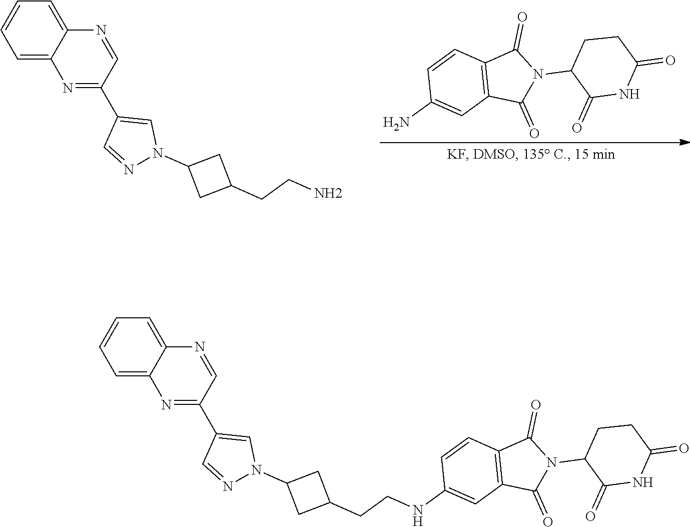 [SMILES: NCCC1CC(n2cc(-c3cnc4ccccc4n3)cn2)C1 ; Nc1ccc2c(c1)C(=O)N(C1CCC(=O)NC1=O)C2=O ; O=C1CCC(N2C(=O)c3ccc(NCCC4CC(n5cc(-c6cnc7ccccc7n6)cn5)C4)cc3C2=O)C(=O)N1]

To a solution of 2-(3-(4-(quinoxalin-2-yl)-1H-pyrazol-1-yl)cyclobutyl)ethan-1-amine (40 mg, 0.14 mmol) in DMSO (1 mL) were added 2-(2,6-dioxopiperidin-3-yl)-5-fluoroisoindoline-1,3-dione (76 mg, 0.28 mmol) and KF (24 mg, 0.42 mmol). After the mixture was irradiated at 135° C. in the microwave reactor for 15 min, it was purified by reverse phase chromatography (MeOH:H₂O=1:1) to provide the title compound (7 mg, yield: 9%) as yellow solid. MS (ESI) m/z=550.4 [M+H]⁺.

### Example 520. 5-((6-(4-(7-Cyclopropylquinoxalin-2-yl)-1H-pyrazol-1-yl)hexyl)amino)-2-(2,6-dioxopiperidin-3-yl)isoindoline-1,3-dione (GS-562)

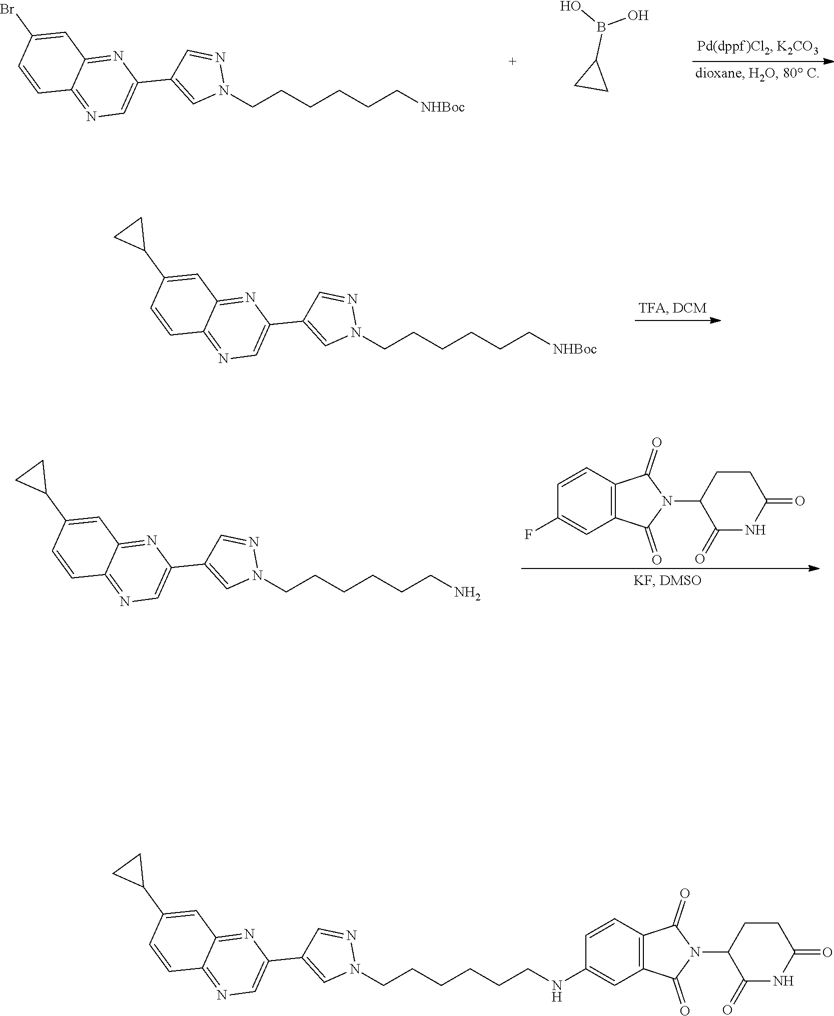 [SMILES: CC(C)(C)OC(=O)NCCCCCCn1cc(-c2cnc3ccc(Br)cc3n2)cn1 ; OB(O)C1CC1 ; CC(C)(C)OC(=O)NCCCCCCn1cc(-c2cnc3ccc(C4CC4)cc3n2)cn1 ; NCCCCCCn1cc(-c2cnc3ccc(C4CC4)cc3n2)cn1 ; O=C1CCC(N2C(=O)c3ccc(F)cc3C2=O)C(=O)N1 ; O=C1CCC(N2C(=O)c3ccc(NCCCCCCn4cc(-c5cnc6ccc(C7CC7)cc6n5)cn4)cc3C2=O)C(=O)N1]

Step 1. Synthesis of tert-butyl (6-(4-(7-cyclopropylquinoxalin-2-yl)-1H-pyrazol-1-yl)hexyl)carbamate

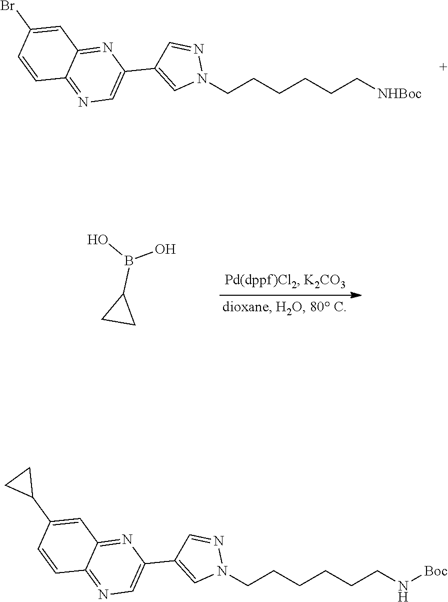 [SMILES: CC(C)(C)OC(=O)NCCCCCCn1cc(-c2cnc3ccc(Br)cc3n2)cn1 ; OB(O)C1CC1 ; CC(C)(C)OC(=O)NCCCCCCn1cc(-c2cnc3ccc(C4CC4)cc3n2)cn1]

A solution of tert-butyl (6-(4-(7-bromoquinoxalin-2-yl)-1H-pyrazol-1-yl)hexyl)carbamate (see step 2 of GS-529 for synthesis, 40 mg, 84.32 μmol), cyclopropylboronic acid (10.86 mg, 126.48 μmol), Pd(dppf)Cl₂ (6.17 mg, 8.43 μmol) and K₂CO₃ (23.31 mg, 168.64 μmol) in dioxane (2 mL) and water (0.5 mL) was stirred at 80° C. under N₂ for 2 h. The mixture was concentrated, and the resulting residue was purified by silica gel chromatography to provide the title compound (30 mg, yield: 82%).

Step 2 to 3. Synthesis of 5-((6-(4-(7-cyclopropylquinoxalin-2-yl)-1H-pyrazol-1-yl)hexyl)amino)-2-(2,6-dioxopiperidin-3-yl)isoindoline-1,3-dione

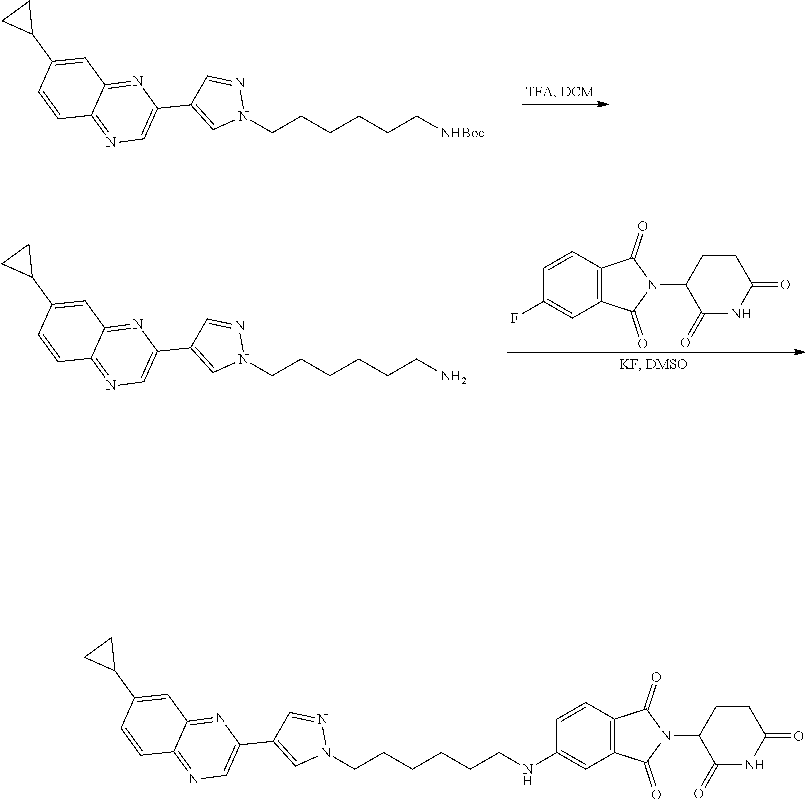 [SMILES: CC(C)(C)OC(=O)NCCCCCCn1cc(-c2cnc3ccc(C4CC4)cc3n2)cn1 ; NCCCCCCn1cc(-c2cnc3ccc(C4CC4)cc3n2)cn1 ; O=C1CCC(N2C(=O)c3ccc(F)cc3C2=O)C(=O)N1 ; O=C1CCC(N2C(=O)c3ccc(NCCCCCCn4cc(-c5cnc6ccc(C7CC7)cc6n5)cn4)cc3C2=O)C(=O)N1]

GS-562 was synthesized following the similar procedures as described for GS-498 (1.10 mg). MS (ESI) m/z=592.5 [M+H]⁺.

### Example 521. 5-((3-(3-(4-(7-(Azetidin-3-yl)quinoxalin-2-yl)-1H-pyrazol-1-yl)cyclobutyl)propyl)amino)-2-(2,6-dioxopiperidin-3-yl)isoindoline-1,3-dione (GS-563)

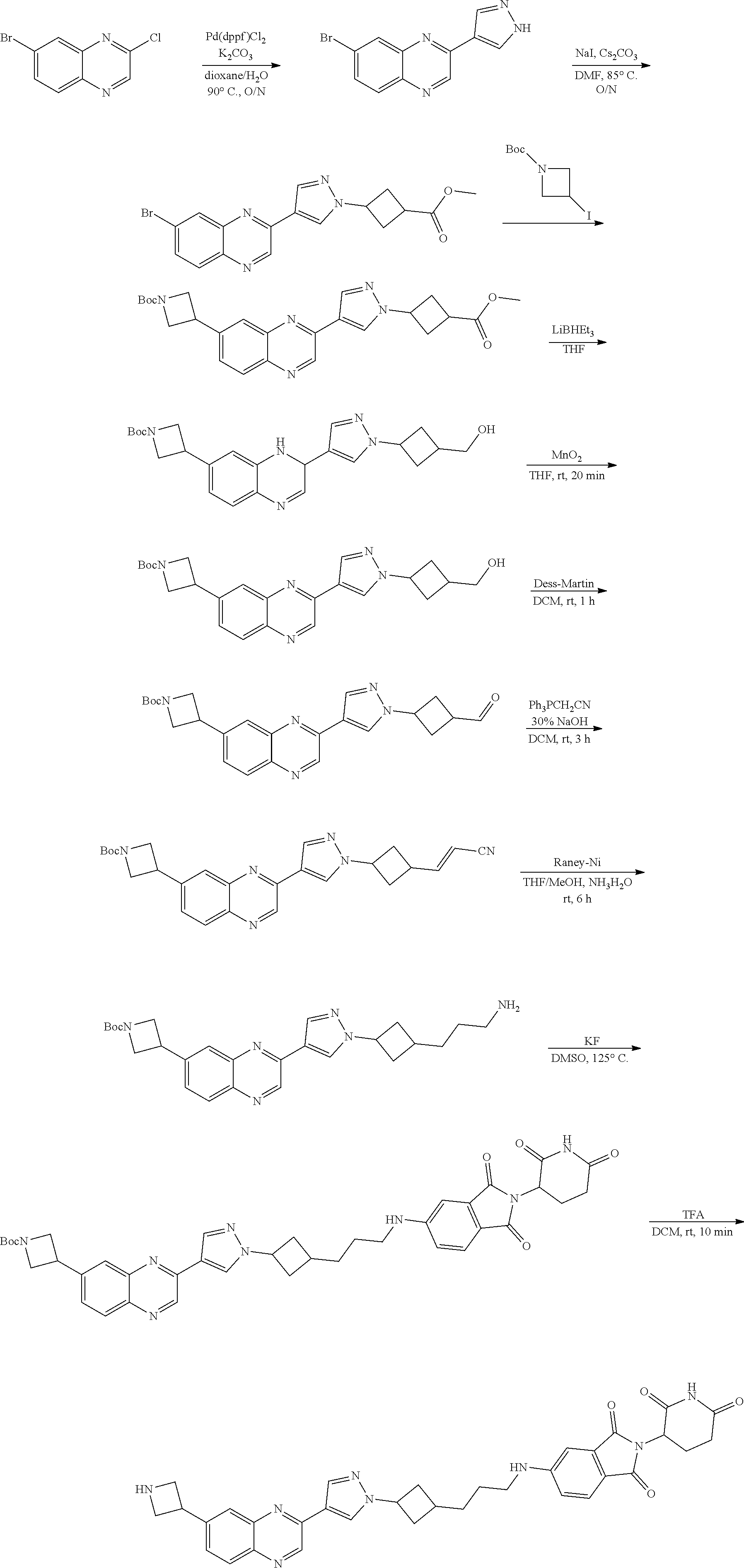

Step 1. Synthesis of 7-bromo-2-(1H-pyrazol-4-yl)quinoxaline

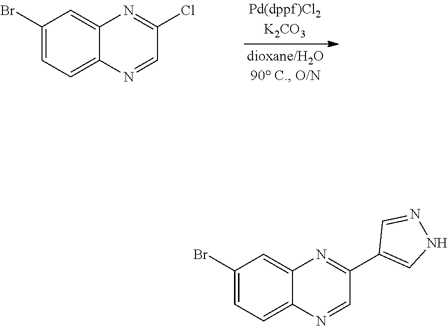 [SMILES: Clc1cnc2ccc(Br)cc2n1 ; Brc1ccc2ncc(-c3cn[nH]c3)nc2c1]

To a mixture of 7-bromo-2-chloro-quinoxaline (2.0 g, 8.2 mmol) and 4-(4,4,5,5-tetramethyl-1,3,2-dioxaborolan-2-yl)-1H-pyrazole (2.39 g, 12.3 mmol) in dioxane (20 mL) and water (5 mL) were added AcOK (1.61 g, 16.4 mmol) and Pd(dppf)Cl₂ (600 mg, 821 μmol). The reaction mixture was stirred at 90° C. overnight. The reaction was quenched with water, and extracted with EtOAc. The organic layer was washed with water, dried over Na₂SO₄, filtered, and concentrated. The resulting residue was purified by reverse phase chromatography (0-70% MeCN in H₂O) to provide the title compound (880 mg, yield: 39%) as brown solid. MS (ESI) m/z=277.0 [M+H]⁺.

Step 2. Synthesis of methyl 3-(4-(7-bromoquinoxalin-2-yl)-1H-pyrazol-1-yl)cyclobutane-1-carboxylate

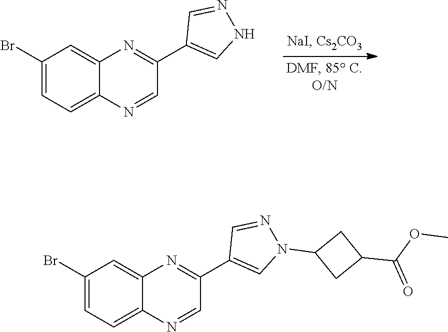 [SMILES: Brc1ccc2ncc(-c3cn[nH]c3)nc2c1 ; COC(=O)C1CC(n2cc(-c3cnc4ccc(Br)cc4n3)cn2)C1]

To a mixture of 7-bromo-2-(1H-pyrazol-4-yl)quinoxaline (880 mg, 3.2 mmol) and methyl 3-(p-tolylsulfonyloxy)cyclobutanecarboxylate (see step 1 of GS-579 for synthesis, 1.36 g, 4.8 mmol) in DMF (10 mL) were added NaI (479 mg, 3.2 mmol) and Cs₂CO₃ (1.04 g, 3.2 mmol). The reaction mixture was stirred at 85° C. overnight. Then, the mixture was quenched with water, and extracted with EtOAc. The organic phase was washed with water, dried over Na₂SO₄, filtered, and concentrated. The resulting residue was purified by reverse phase chromatography (0-70% MeCN in H₂O) to provide the title compound (920 mg, yield: 74%) as bright oil. MS (ESI) m/z=389.1 [M+H]⁺.

Step 3. Synthesis of tert-butyl 3-(3-(1-(3-(methoxycarbonyl)cyclobutyl)-1H-pyrazol-4-yl)quinoxalin-6-yl)azetidine-1-carboxylate

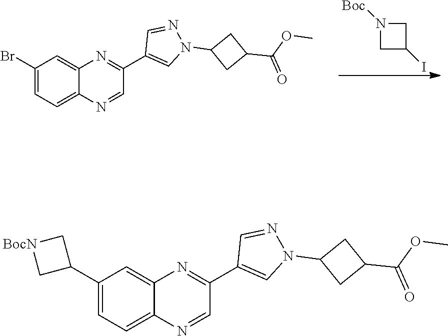 [SMILES: COC(=O)C1CC(n2cc(-c3cnc4ccc(Br)cc4n3)cn2)C1 ; CC(C)(C)OC(=O)N1CC(I)C1 ; COC(=O)C1CC(n2cc(-c3cnc4ccc(C5CN(C(=O)OC(C)(C)C)C5)cc4n3)cn2)C1]

To a mixture of Zn (570 mg, 8.8 mmol) in DMF (10 mL) was added 1,2-dibromoethane (41 mg, 219 μmol) under Ar. The mixture was stirred at 75° C. for 10 min. After the reaction was cooled to rt, 1,2-dibromoethane (41 mg, 219 μmol) was added. After the reaction mixture was stirred at rt for 1 h, tert-butyl 3-iodoazetidine-1-carboxylate (1.24 g, 4.4 mmol) was added. After the solution was stirred at 45° C. for 1 h, methyl 3-(4-(7-bromoquinoxalin-2-yl)-1H-pyrazol-1-yl)cyclobutane-1-carboxylate (850 mg, 2.2 mmol) was added. The mixture was stirred at 70° C. overnight, before it was quenched with aq. NH₄Cl solution, and extracted with EtOAc. The organic phase was washed with water, dried over Na₂SO₄, filtered, and concentrated. The resulting residue was purified by reverse phase chromatography (0-70% MeCN in H₂O) to provide the title compound (700 mg, yield: 69%) as bright oil. MS (ESI) m/z=464.3 [M+H]⁺.

Step 4. Synthesis of tert-butyl 3-(3-(1-(3-(hydroxymethyl)cyclobutyl)-1H-pyrazol-4-yl)-3,4-dihydroquinoxalin-6-yl)azetidine-1-carboxylate

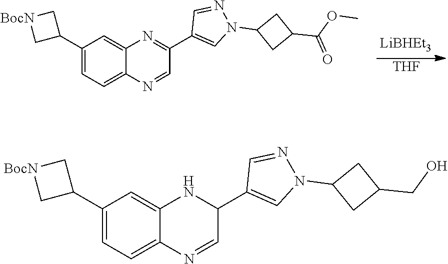 [SMILES: COC(=O)C1CC(n2cc(-c3cnc4ccc(C5CN(C(=O)OC(C)(C)C)C5)cc4n3)cn2)C1 ; CC(C)(C)OC(=O)N1CC(c2ccc3c(c2)NC(c2cnn(C4CC(CO)C4)c2)C=N3)C1 ; "THF"]

To a solution of tert-buty 3-(3-(1-(3-(hydroxymethyl)cyclobutyl)-1H-pyrazol-4-yl)quinoxalin-6-yl)azetidine-1-carboxylate (700 mg, 1.5 mmol) in THF (10 mL) was added lithium triethylborohydride (319 mg, 3.0 mmol) at 0° C. under N₂. After the reaction mixture was stirred at rt for 20 min, it was quenched with water, and extracted with EtOAc. The organic phase was washed with water, dried over Na₂SO₄, filtered, and concentrated. The resulting residue was purified by reverse phase chromatography (0-70% MeCN in H₂O) to provide the title compound (500 mg, yield: 75%) as yellow solid. MS (ESI) m/z=438.3 [M+H]⁺.

Step 5. Synthesis of tert-butyl 3-(3-(1-(3-(hydroxymethyl)cyclobutyl)-1H-pyrazol-4-yl)quinoxalin-6-yl)azetidine-1-carboxylate

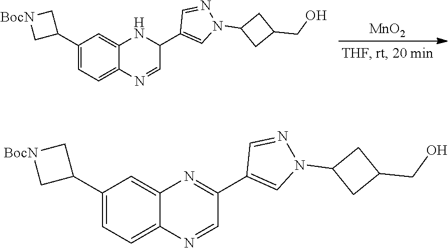

To a solution of tert-butyl 3-(3-(1-(3-(hydroxymethyl)cyclobutyl)-1H-pyrazol-4-yl)-3,4-dihydroquinoxalin-6-yl)azetidine-1-carboxylate (500 mg, 1.1 mmol) in DCM (5 mL) was added MnO₂ (199 mg, 2.3 mmol). The reaction mixture was stirred at rt for 20 min. After filtration, the filtrate was concentrated. The resulting residue was purified by reverse phase chromatography (0-70% MeCN in H₂O) to provide the title compound (300 mg, yield: 60%) as yellow oil. MS (ESI) m/z=436.3 [M+H]⁺.

Step 6. Synthesis of tert-butyl 3-(3-(1-(3-formylcyclobutyl)-1H-pyrazol-4-yl)quinoxalin-6-yl)azetidine-1-carboxylate

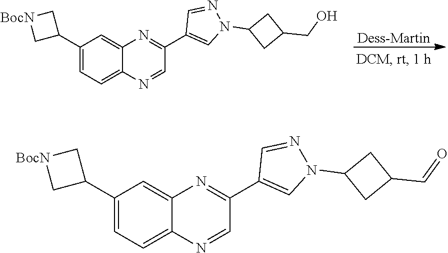 [SMILES: CC(C)(C)OC(=O)N1CC(c2ccc3ncc(-c4cnn(C5CC(CO)C5)c4)nc3c2)C1 ; "Dess-Martin" ; CC(C)(C)OC(=O)N1CC(c2ccc3ncc(-c4cnn(C5CC(C=O)C5)c4)nc3c2)C1]

To a solution of tert-butyl 3-(3-(1-(3-(hydroxymethyl)cyclobutyl)-1H-pyrazol-4-yl)quinoxalin-6-yl)azetidine-1-carboxylate (300 mg, 688 μmol) in DCM (6 mL) was added Dess-Martin Periodinane (584 mg, 1.38 mmol) at 0° C. The reaction mixture was stirred at rt for 1 h, before it was quenched with water. The resulting mixture was extracted with DCM. The organic phase was washed with water, dried over Na₂SO₄, filtered, and concentrated. The resulting residue was purified by reverse phase chromatography (0-70% MeCN in H₂O) to provide the title compound (250 mg, yield: 84%) as yellow oil. MS (ESI) m/z=434.3 [M+H]$.

Step 7. Synthesis of tert-butyl-3-(3-(1-(3-(2-cyanovinyl)cyclobutyl)-1H-pyrazol-4-yl)quinoxalin-6-yl)azetidine-1-carboxylate

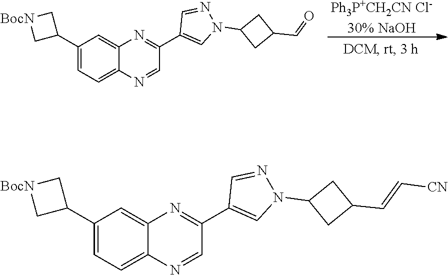 [SMILES: CC(C)(C)OC(=O)N1CC(c2ccc3ncc(-c4cnn(C5CC(C=O)C5)c4)nc3c2)C1 ; "Ph3P+CH2CN Cl-30% NaOH" ; CC(C)(C)OC(=O)N1CC(c2ccc3ncc(-c4cnn(C5CC(/C=C/C#N)C5)c4)nc3c2)C1]

To a solution of tert-butyl 3-(3-(1-(3-formylcyclobutyl)-1H-pyrazol-4-yl)quinoxalin-6-yl)azetidine-1-carboxylate (250 mg, 576 μmol) in DCM (5 mL) were added (cyanomethyl)triphenylphosphonium chloride (214 mg, 634 μmol) and NaOH (30%, 0.3 mL). The reaction mixture was stirred at rt for 2 h, before it was quenched with water. The resulting mixture was extracted with DCM. The organic phase was washed with water, dried over Na₂SO₄, filtered, and concentrated. The resulting residue was purified by reverse phase chromatography (0-70% MeCN in H₂O) to provide the title compound (150 mg, yield: 57%) as bright oil. MS (ESI) m/z=457.3 [M+H]⁺.

Step 8. Synthesis of tert-butyl 3-(3-(1-(3-(3-aminopropyl)cyclobutyl)-1H-pyrazol-4-yl)quinoxalin-6-yl)azetidine-1-carboxylate

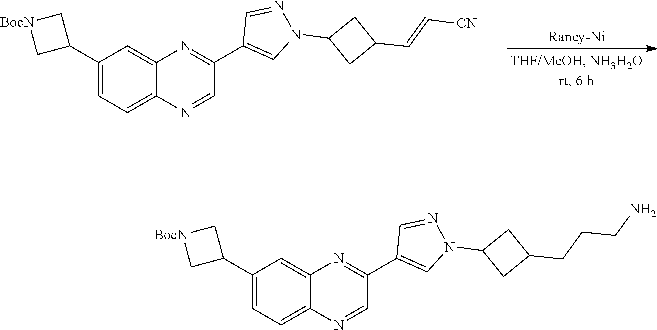 [SMILES: CC(C)(C)OC(=O)N1CC(c2ccc3ncc(-c4cnn(C5CC(/C=C/C#N)C5)c4)nc3c2)C1 ; "Raney-Ni" ; CC(C)(C)OC(=O)N1CC(c2ccc3ncc(-c4cnn(C5CC(CCCN)C5)c4)nc3c2)C1]

To a solution of tert-butyl-3-(3-(1-(3-(2-cyanovinyl)cyclobutyl)-1H-pyrazol-4-yl)quinoxalin-6-yl)azetidine-1-carboxylate (100 mg, 219 μmol) and NH₃·H₂O (0.1 mL) in THF (2 mL) and methanol (2 mL) was added Raney-Ni (25 mg, 219 μmol). The reaction mixture was purged with H₂ three times. After the mixture was stirred at rt for 6 h, it was filtered. The filtrate was concentrated and the resulting residue was purified by reverse phase chromatography (0-70% MeCN in H₂O) to provide the title compound (80 mg, yield: 77%) as bright oil. MS (ESI) m/z=463.3 [M+H]⁺.

Step 9. Synthesis of tert-butyl 3-(3-(1-(3-(3-((2-(2,6-dioxopiperidin-3-yl)-1,3-dioxoisoindolin-5-yl)amino)propyl)cyclobutyl)-1H-pyrazol-4-yl)quinoxalin-6-yl)azetidine-1-carboxylate

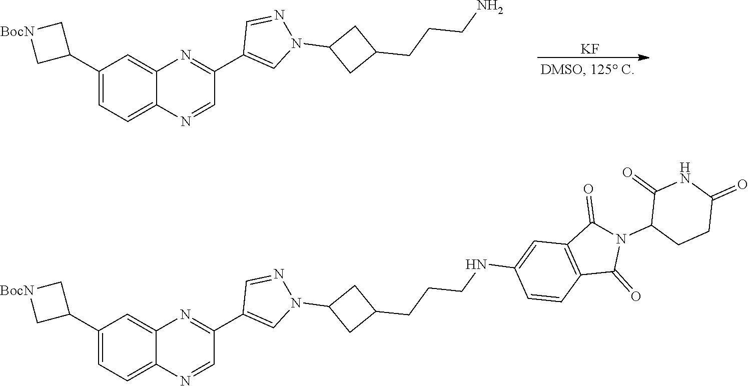 [SMILES: CC(C)(C)OC(=O)N1CC(c2ccc3ncc(-c4cnn(C5CC(CCCN)C5)c4)nc3c2)C1 ; "KF" ; CC(C)(C)OC(=O)N1CC(c2ccc3ncc(-c4cnn(C5CC(CCCNc6ccc7c(c6)C(=O)N(C6CCC(=O)NC6=O)C7=O)C5)c4)nc3c2)C1]

To a mixture of tert-butyl 3-(3-(1-(3-(3-aminopropyl)cyclobutyl)-1H-pyrazol-4-yl)quinoxalin-6-yl)azetidine-1-carboxylate (80 mg, 173 μmol) and 2-(2,6-dioxo-3-piperidyl)-5-fluoro-isoindoline-1,3-dione (96 mg, 346 μmol) in DMSO (2 mL) was added KF (20 mg, 346 μmol). The reaction mixture was irradiated at 125° C. for 20 min in the microwave reactor. The mixture was purified by reverse phase chromatography (0-70% MeCN in H₂O) to provide the title compound (20 mg, yield: 16%) as yellow solid. MS (ESI) m/z=719.3 [M+H]⁺.

Step 10. Synthesis of 5-((3-(3-(4-(7-(azetidin-3-yl)quinoxalin-2-yl)-1H-pyrazol-1-yl)cyclobutyl)propyl)amino)-2-(2,6-dioxopiperidin-3-yl)isoindoline-1,3-dione

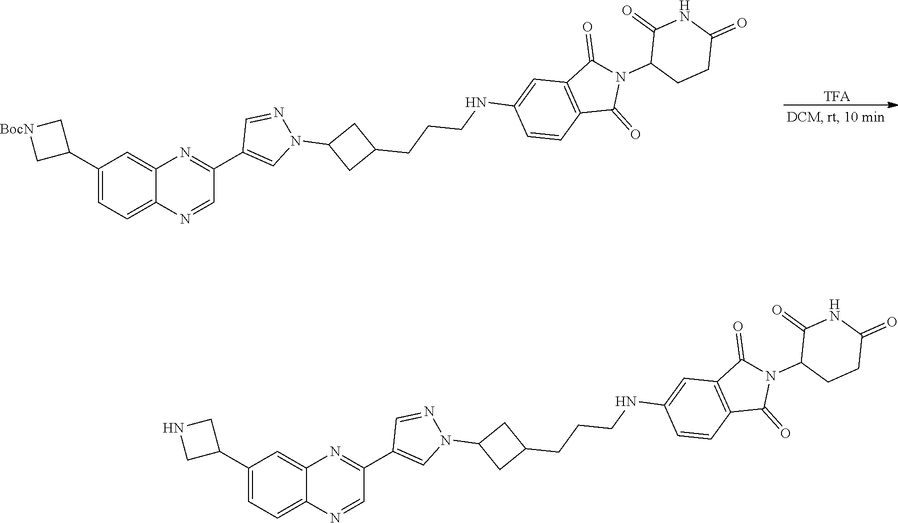 [SMILES: "TFA" ; CC(C)(C)OC(=O)N1CC(c2ccc3ncc(-c4cnn(C5CC(CCCNc6ccc7c(c6)C(=O)N(C6CCC(=O)NC6=O)C7=O)C5)c4)nc3c2)C1 ; O=C1CCC(N2C(=O)c3ccc(NCCCC4CC(n5cc(-c6cnc7ccc(C8CNC8)cc7n6)cn5)C4)cc3C2=O)C(=O)N1]

A solution of tert-butyl 3-(3-(1-(3-(3-((2-(2,6-dioxopiperidin-3-yl)-1,3-dioxoisoindolin-5-yl)amino)propyl)cyclobutyl)-1H-pyrazol-4-yl)quinoxalin-6-yl)azetidine-1-carboxylate (30 mg, 42 μmol) in DCM (2 mL) was added TFA (1 mL). The reaction mixture was stirred at rt for 10 min, before it was purified by reverse phase chromatography (0-70% MeCN in H₂O) to provide the title compound (20 mg, yield: 65%) as yellow solid. MS (ESI) m/z=619.6 [M+H]⁺.

### Example 522. 2-(2,6-Dioxopiperidin-3-yl)-5-((2-(6-(4-(quinoxalin-2-yl)-1H-pyrazol-1-yl)spiro[3.3]heptan-2-yl)ethyl)amino)isoindoline-1,3-dione (GS-564)

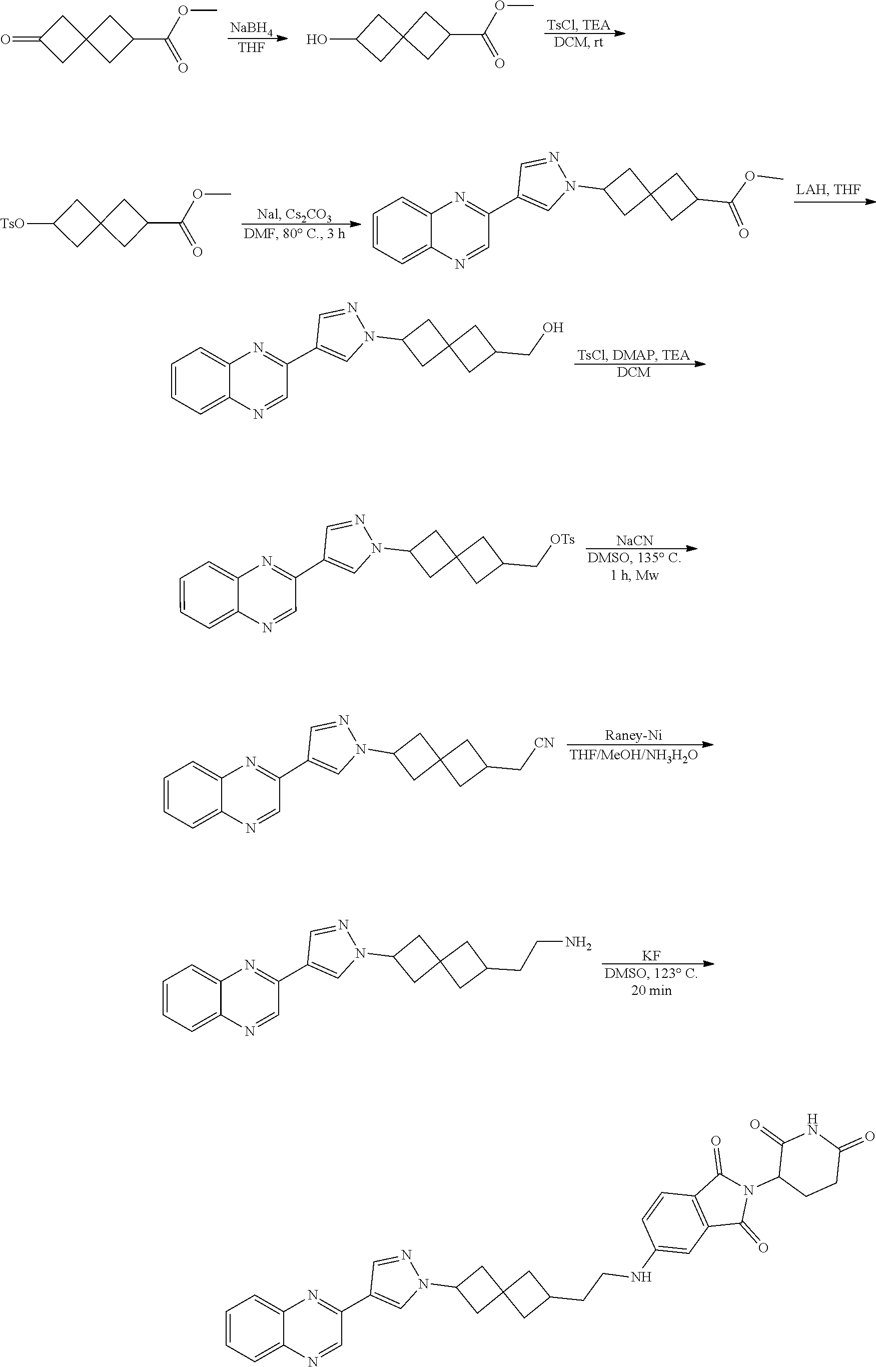

Step 1. Synthesis of methyl 6-hydroxyspiro[3.3]heptane-2-carboxylate

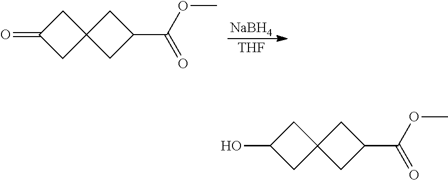

To a solution of methyl 2-oxospiro[3.3]heptane-6-carboxylate (500 mg, 3.0 mmol) in THF (10 mL) was added NaBH₄ (169 mg, 4.5 mmol). The reaction mixture was stirred at rt for 3 h. The mixture was purified by silica gel chromatography (petroleum ether:EtOAc=1:0 to 1:1) to provide the title compound (350 mg, yield: 69%) as bright oil. MS (ESI) m/z=171.2 [M+H]⁺.

Step 2 to 5. Synthesis of (6-(4-(quinoxalin-2-yl)-1H-pyrazol-1-yl)spiro[3.3]heptan-2-yl)methyl 4-methylbenzenesulfonate

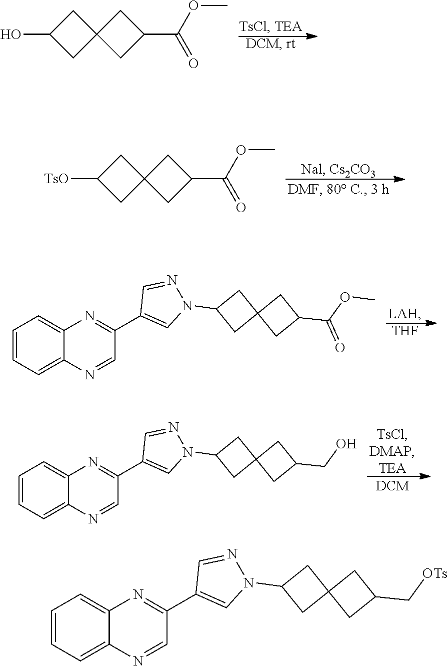 [SMILES: COC(=O)C1CC2(CC(O)C2)C1 ; COC(=O)C1CC2(CC(OS(=O)(=O)c3ccc(C)cc3)C2)C1 ; COC(=O)C1CC2(C1)CC(n1cc(-c3cnc4ccccc4n3)cn1)C2 ; OCC1CC2(C1)CC(n1cc(-c3cnc4ccccc4n3)cn1)C2 ; Cc1ccc(S(=O)(=O)OCC2CC3(C2)CC(n2cc(-c4cnc5ccccc5n4)cn2)C3)cc1]

The title compound was synthesized following the similar procedures as described for steps 1 to 4 of GS-561 (290 mg) as bright oil. MS (ESI) m/z=475.3 [M+H]⁺.

Step 6. Synthesis of 2-(6-(4-(quinoxalin-2-yl)-1H-pyrazol-1-yl)spiro[3.3]heptan-2-yl)acetonitrile

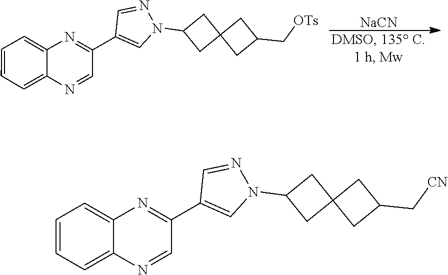 [SMILES: Cc1ccc(S(=O)(=O)OCC2CC3(C2)CC(n2cc(-c4cnc5ccccc5n4)cn2)C3)cc1 ; N#CCC1CC2(C1)CC(n1cc(-c3cnc4ccccc4n3)cn1)C2]

A mixture of (6-(4-(quinoxalin-2-yl)-1H-pyrazol-1-yl)spiro[3.3]heptan-2-yl)methyl 4-methylbenzenesulfonate (290 mg, 0.6 mmol) and NaCN (120 mg, 1.5 mmol) in DMSO (2.5 mL) was irradiated at 130° C. in the microwave reactor for 1 h. The reaction was quenched with water, and extracted with EtOAc. The organic phase was washed with water, dried over Na₂SO₄, filtered, and concentrated. The resulting residue was purified by reverse phase chromatography (0-70% MeCN in H₂O) to provide the title compound (250 mg, yield: 57%) as yellow oil. MS (ESI) m/z=330.3 [M+H]⁺.

Step 7. Synthesis of 2-(6-(4-(quinoxalin-2-yl)-1H-pyrazol-1-yl)spiro[3.3]heptan-2-yl)ethan-1-amine

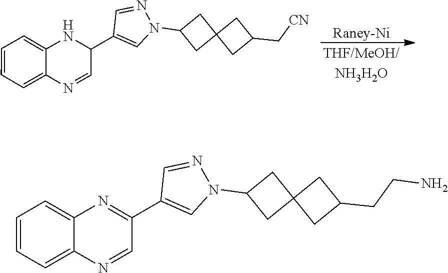 [SMILES: N#CCC1CC2(C1)CC(n1cc(C3C=Nc4ccccc4N3)cn1)C2 ; NCCC1CC2(C1)CC(n1cc(-c3cnc4ccccc4n3)cn1)C2]

To a solution of 2-(6-(4-(1,2-dihydroquinoxalin-2-yl)-1H-pyrazol-1-yl)spiro[3.3]heptan-2-yl)acetonitrile (70 mg, 212 μmol) in NH₃·H₂O (0.2 mL), methanol (3 mL) and THF (1 mL) was added Raney-Ni (200 mg, 212 μmol). The reaction mixture was purged with H₂ three times. After the mixture was stirred at rt for 2 h, it was filtered. The filtrate was concentrated and the resulting residue was purified by reverse phase chromatography (0-70% MeCN in H₂O) to provide the title compound (50 mg, yield: 70%) as bright oil. MS (ESI) m/z=334.3 [M+H]⁺.

Step 8. Synthesis of 2-(2,6-dioxopiperidin-3-yl)-5-((2-(6-(4-(quinoxalin-2-yl)-1H-pyrazol-1-yl)spiro[3.3]heptan-2-yl)ethyl)amino)isoindoline-1,3-dione

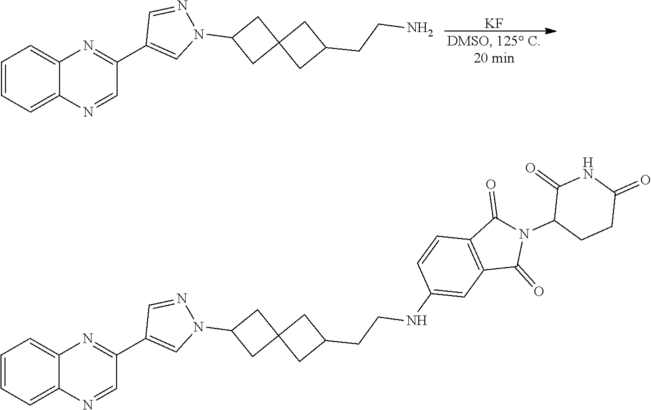 [SMILES: NCCC1CC2(C1)CC(n1cc(-c3cnc4ccccc4n3)cn1)C2 ; O=C1CCC(N2C(=O)c3ccc(NCCC4CC5(C4)CC(n4cc(-c6cnc7ccccc7n6)cn4)C5)cc3C2=O)C(=O)N1]

To a mixture of 2-(6-(4-(quinoxalin-2-yl)-1H-pyrazol-1-yl)spiro[3.3]heptan-2-yl)ethan-1-amine (50 mg, 150 μmol) and 2-(2,6-dioxo-3-piperidyl)-5-fluoro-isoindoline-1,3-dione (41 mg, 150 μmol) in DMSO (1.5 mL) was added KF (17 mg, 300 μmol). The reaction mixture was irradiated at 125° C. in the microwave reactor for 20 min. The reaction was purified by reverse phase chromatography (0-70% MeCN in H₂O) to provide the title compound (4.4 mg, yield: 5%) as green solid. MS (ESI) m/z=590.5 [M+H]⁺.

### Example 523. 2-(2,6-Dioxopiperidin-3-yl)-4-((3-(3-(4-(quinoxalin-2-yl)-1-pyrazol-1-yl)cyclobutyl)propyl)amino)isoindoline-1,3-dione (GS-565)

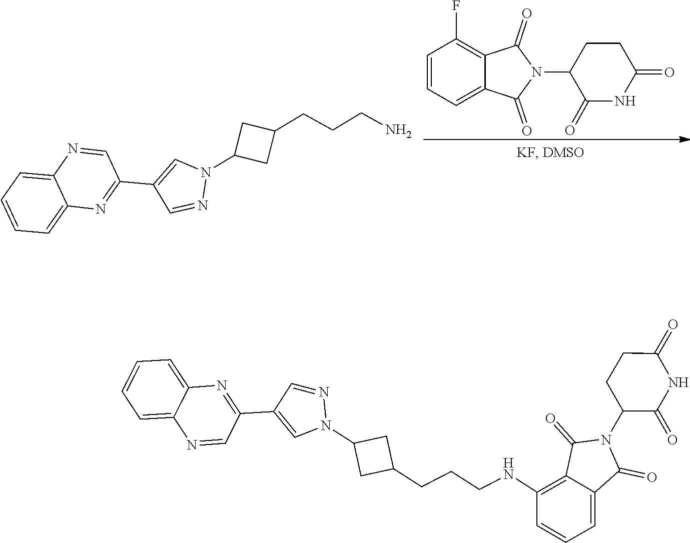

A mixture of 3-(3-(4-(quinoxalin-2-yl)-1H-pyrazol-1-yl)cyclobutyl)propan-1-amine (see step 5 of GS-107 for synthesis, 38 mg, 92.72 μmol), 2-(2,6-dioxo-3-piperidyl)-4-fluoro-isoindoline-1,3-dione (60 mg, 217.22 μmol) and KF (10 mg, 172.13 μmol) in DMSO (2 mL) was irradiated at 125° C. in the microwave reactor for 20 min. After the mixture was cooled to it, it was purified by reverse phase chromatography to provide the title compound (6 mg, yield: 11%) as yellow solid. MS (ESI) m/z=564.4 [M+H]⁺.

### Example 524. 5-((3-(3-(4-(7-(Azetidin-3-ylmethyl)quinoxalin-2-yl)-1H-pyrazol-1-yl)cyclobutyl)propyl)amino)-2-(2,6-dioxopiperidin-3-yl)isoindoline-1,3-dione (GS-566)

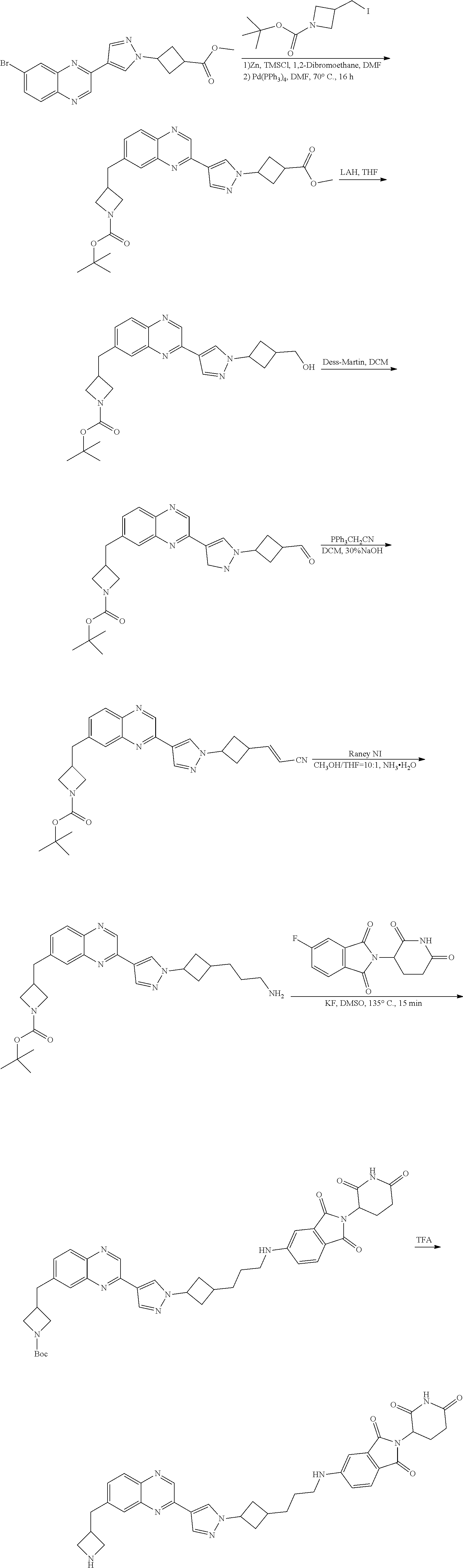

Step 1. Synthesis of tert-butyl 3-((3-(1-(3-(methoxycarbonyl)cyclobutyl)-1H-pyrazol-4-yl)quinoxalin-6-yl)methyl)azetidine-1-carboxylate

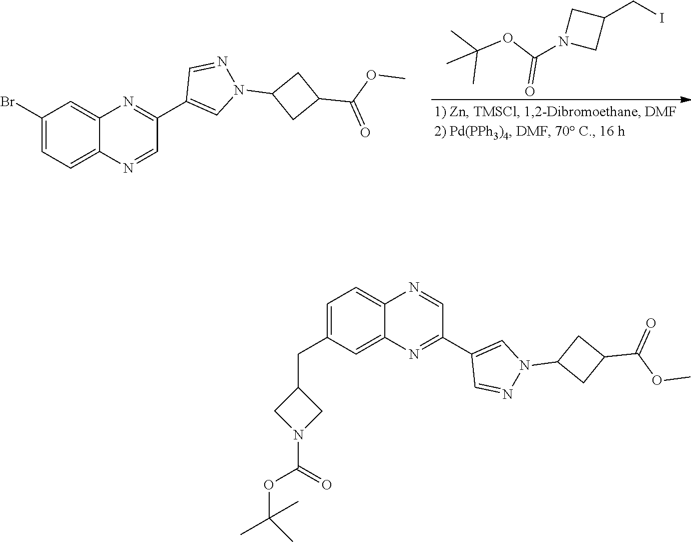

To a mixture of Zn (195 mg, 3 mmol) in DMF (2 ml) was added 1,2-dibromoethane (0.1 mL). After the mixture was stirred at 70° C. for 10 min, TMSCI (0.1 mL) was added at rt. After the mixture was stirred at rt for 1 h, tert-butyl 3-(iodomethyl)azetidine-1-carboxylate (742 mg, 2.5 mmol) was added. The mixture was stirred at 40° C. for 1 h, before Pd(PPh₃)₄ (115 mg, 0.1 mmol), methyl 3-(4-(7-bromoquinoxalin-2-yl)-1H-pyrazol-1-yl)cyclobutane-1-carboxylate (386 mg, 1 mmol) and DMF (3 mL) were added. After the mixture was stirred at 70° C. for 16 h, it was purified by reverse phase chromatography (MeOH:H₂O=2:1) to provide the title compound (310 mg, yield: 65%) as yellow oil. MS (ESI) m/z=478.3 [M+H]⁺.

Step 2. Synthesis of tert-butyl 3-((3-(1-(3-(hydroxymethyl)cyclobutyl)-1H-pyrazol-4-yl)quinoxalin-6-yl)methyl)azetidine-1-carboxylate

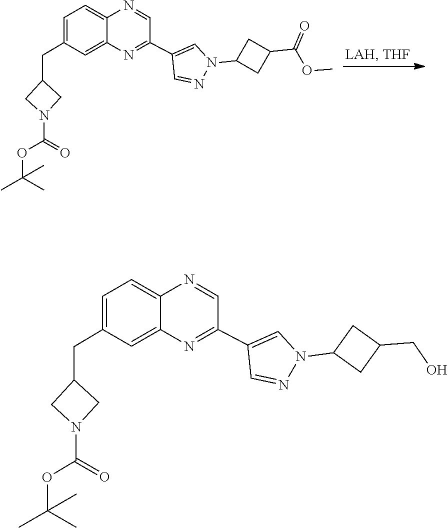

To a solution of tert-butyl 3-((3-(1-(3-(methoxycarbonyl)cyclobutyl)-1H-pyrazol-4-yl)quinoxalin-6-yl)methyl)azetidine-1-carboxylate (310 mg, 0.65 mmol) in THF (20 mL) was added LiAlH₄ (49 mg, 1.3 mmol). After the mixture was stirred at rt for 2 h, 2 drops of water were added. The obtained mixture was stirred for 30 min, filtered, and concentrated to provide the title compound (280 mg, yield: 96%) as yellow oil. MS (ESI) m/z=450.3 [M+H]⁺.

Step 3 to 7. Synthesis of 5-((3-(3-(4-(7-(azetidin-3-ylmethyl)quinoxalin-2-yl)-1H-pyrazol-1-yl)cyclobutyl)propyl)amino)-2-(2,6-dioxopiperidin-3-yl)isoindoline-1,3-dione

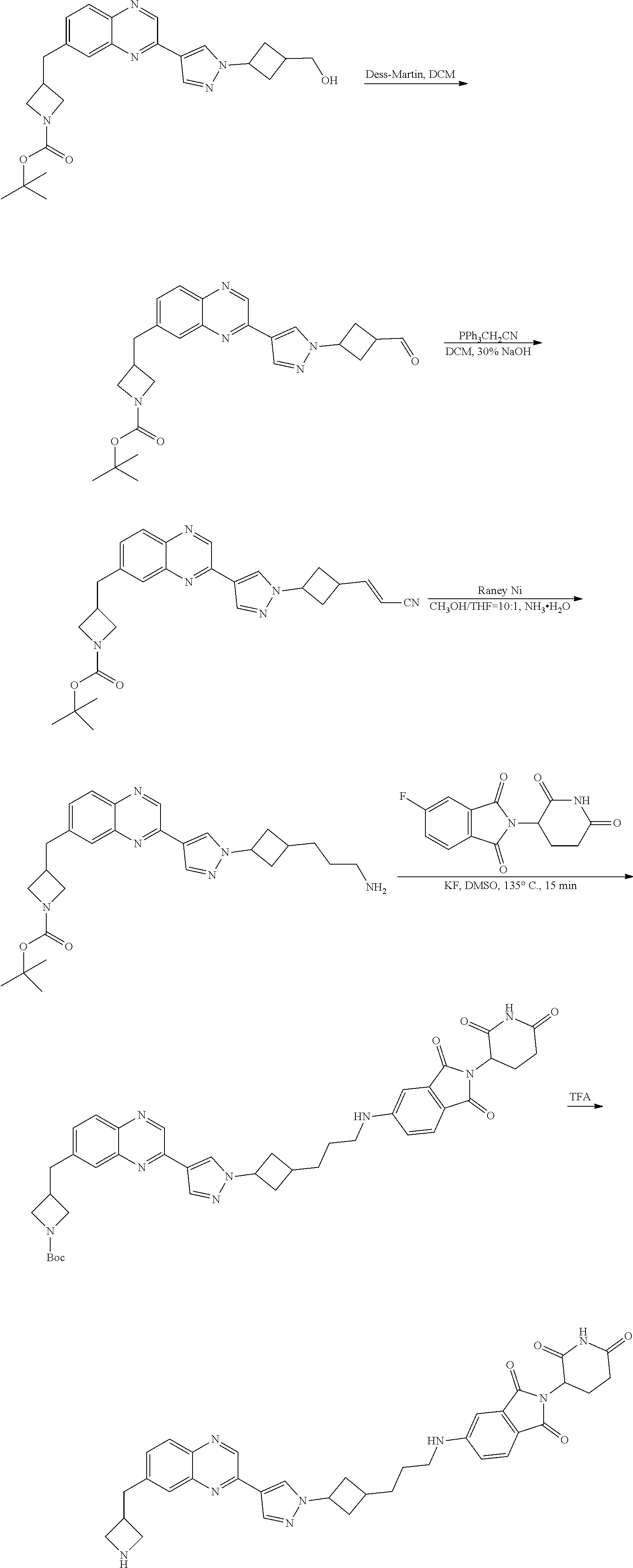

GS-566 was synthesized following the similar procedures as described for GS-563 (5 mg) as yellow solid. MS (ESI) m/z=633.6 [M+H]⁺.

### Example 525. 2-(2,6-Dioxopiperidin-3-yl)-5-((3-(3-(4-(7-(oxetan-3-ylamino)quinoxalin-2-yl)-1H-pyrazol-1-yl)cyclobutyl)propyl)amino)isoindoline-1,3-dione (GS-567)

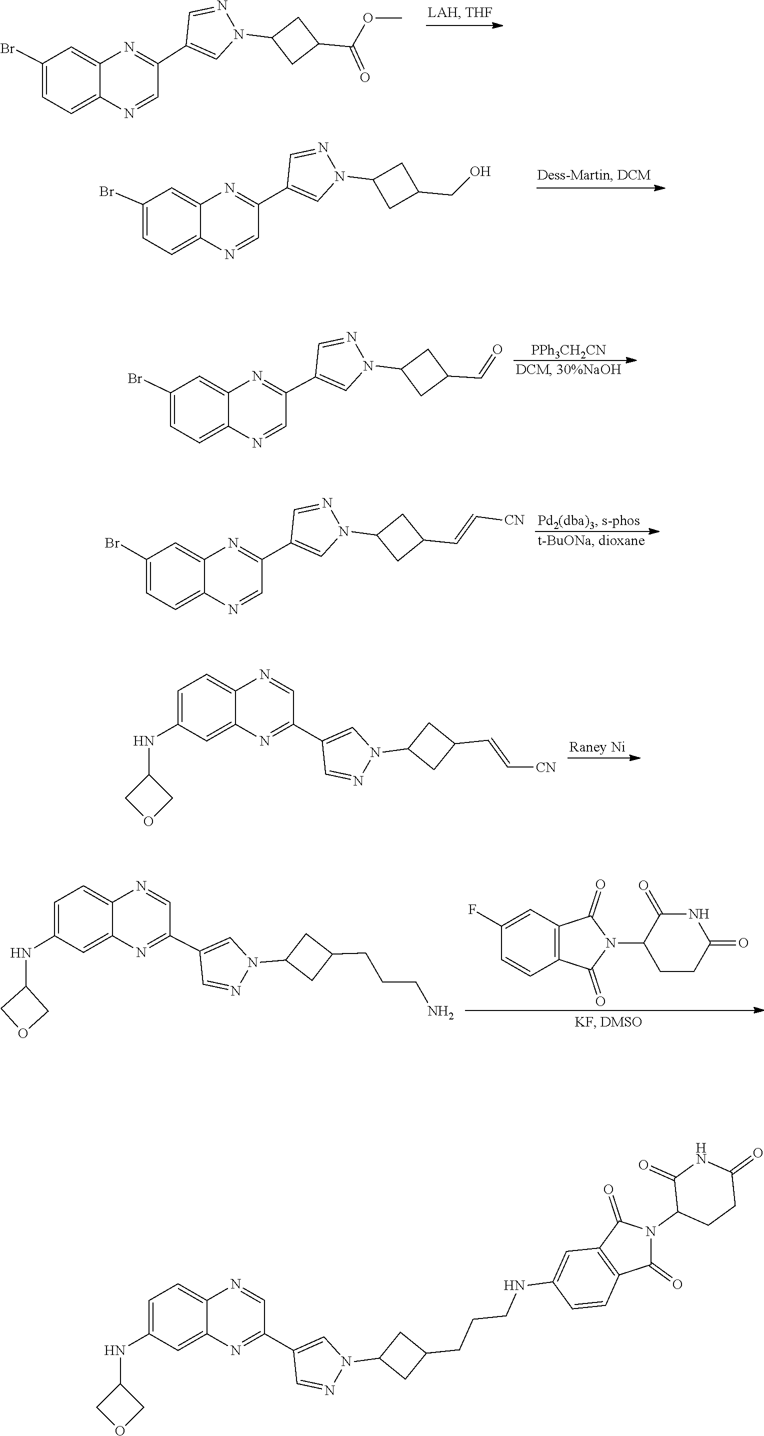

Step 1. Synthesis of (3-(4-(7-bromoquinoxalin-2-yl)-1H-pyrazol-1-yl)cyclobutyl)methanol

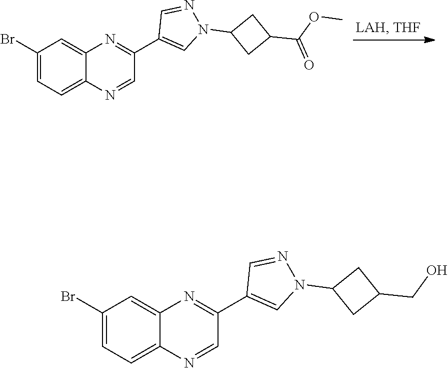 [SMILES: COC(=O)C1CC(n2cc(-c3cnc4ccc(Br)cc4n3)cn2)C1 ; OCC1CC(n2cc(-c3cnc4ccc(Br)cc4n3)cn2)C1]

To a solution of methyl 3-(4-(7-bromoquinoxalin-2-yl)-1H-pyrazol-1-yl)cyclobutane-1-carboxylate (300 mg, 0.78 mmol) in DCM (10 mL) was added LiAlH₄ (64 mg, 1.56 mmol). The mixture was stirred at rt for 16 h, before DCM (20 mL) and NaHSO₃ (100 mg) were added. After the obtained for 30 filtered, concentrated to provide the title compound (260 mg, yield: 81) as yellow oil. MS (ESI) m/z=359.3[M+H]⁺.

Step 2. Synthesis of (3-(4-(7-bromoquinoxalin-2-yl)-1H-pyrazol-1-yl)cyclobutane-1-carbaldehyde

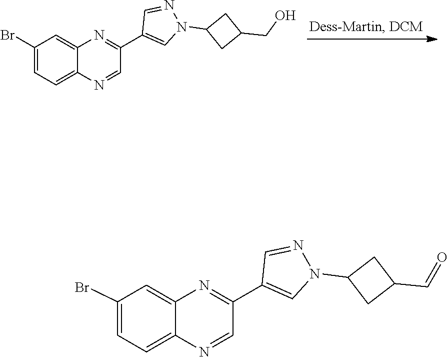 [SMILES: OCC1CC(n2cc(-c3cnc4ccc(Br)cc4n3)cn2)C1 ; O=CC1CC(n2cc(-c3cnc4ccc(Br)cc4n3)cn2)C1]

To a solution of (3-(4-(7-bromoquinoxalin-2-yl)-1H-pyrazol-1-yl)cyclobutyl)methanol (260 mg, 0.73 mmol) in DCM (10 mL) was added Dess-Martin Periodinane (464 mg, 1.09 mmol). The mixture was stirred at rt for 16 h, before DCM (20 mL) and NaHSO₃ (100 mg) were added. After the obtained for 30 min, filtered, and concentrated to provide the title compound (20 mg, yield: 81%) as yellow oil. MS (ESI) m/z=35.3 [M+H]⁺.

Step 3. Synthesis of 3-(3-(4-(7-bromoquinoxalin-2-yl)-1H-pyrazol-1-yl)cyclobutyl)acrylonitrile

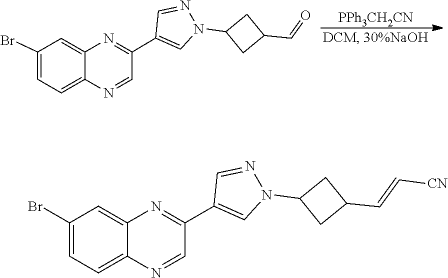 [SMILES: O=CC1CC(n2cc(-c3cnc4ccc(Br)cc4n3)cn2)C1 ; N#C/C=C/C1CC(n2cc(-c3cnc4ccc(Br)cc4n3)cn2)C1]

To a solution of 3-(4-(7-bromoquinoxalin-2-yl)-1H-pyrazol-1-yl)cyclobutane-1-carbaldehyde (210 mg, 0.59 mmol) in DCM (10 mL) were added (cyanomethyl)triphenylphosphonium chloride (218 mg, 0.65 mmol) and 30% NaOH (3 drops). After the mixture was stirred at rt for 30 min. it was diluted with water (20 mL). The mixture was extracted with DCM (10 mL). The organic phase was washed with brine (10 mL), and concentrated. The resulting residue was purified by silica gel chromatography (petroleum ether:EtOAc=2:1) to provide the title compound (120 mg, yield: 54%) as colorless oil. MS (ESI) m/z=380.3 [M+H]⁺.

Step 4. Synthesis of 3-(3-(4-(7-(oxetan-3-ylamino)quinoxalin-2-yl)-1H-pyrazol-1-yl)cyclobutyl)acrylonitrile

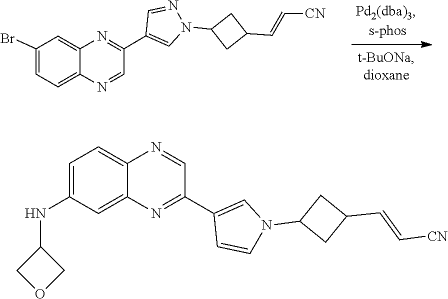 [SMILES: N#C/C=C/C1CC(n2cc(-c3cnc4ccc(Br)cc4n3)cn2)C1 ; N#C/C=C/C1CC(n2ccc(-c3cnc4ccc(NC5COC5)cc4n3)c2)C1]

To a solution of 3-(3-(4-(7-bromoquinoxalin-2-yl)-1H-pyrazol-1-yl)cyclobutyl)acrylonitrile (90 mg, 0.23 mmol) in dioxane (2 mL) were added oxetan-3-amine (33 mg, 0.46 mmol), Pd₂(dba)₃ (21 mg, 0.02 mmol), s-phos (18 mg, 0.04 mmol) and t-BuONa (44 mg, 0.46 mmol). The mixture was stirred at 90° C. for 1 h, before it was purified by reverse phase chromatography (MeOH:H₂O=1:1) to provide the title compound (30 mg, yield: 34%) as yellow oil.

Step 5. Synthesis of 3-(1-(3-(3-aminopropyl)cyclobutyl)-1H-pyrazol-4-yl)-N-(oxetan-3-yl)quinoxalin-6-amine

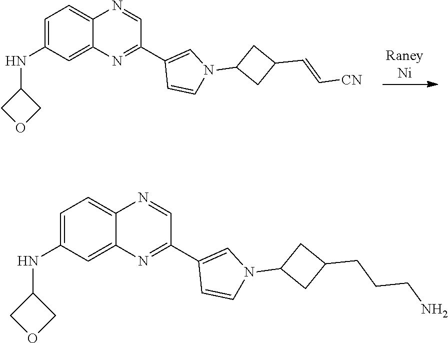 [SMILES: N#C/C=C/C1CC(n2ccc(-c3cnc4ccc(NC5COC5)cc4n3)c2)C1 ; NCCCC1CC(n2ccc(-c3cnc4ccc(NC5COC5)cc4n3)c2)C1]

To a solution of 3-(3-(4-(7-(oxetan-3-ylamino)quinoxalin-2-yl)-1H-pyrazol-1-yl)cyclobutyl)acrylonitrile (30 mg, 0.08 mmol) in MeOH and THF (CH₃OH:THF=10:1, 5 mL) were added NH₃·H₂O (1 drop) and Raney Ni (3 mg). After the mixture was stirred at rt for 16 h, it was filtered. The filtrate was concentrated to provide the title compound (20 mg, yield: 67%). MS (ESI) m/z=379.3 [M+H]⁺.

Step 6. Synthesis of 2-(2,6-dioxopiperidin-3-yl)-5-((3-(3-(4-(7-(oxetan-3-ylamino)quinoxalin-2-yl)-1H-pyrazol-1-yl)cyclobutyl)propyl)amino)isoindoline-1,3-dione

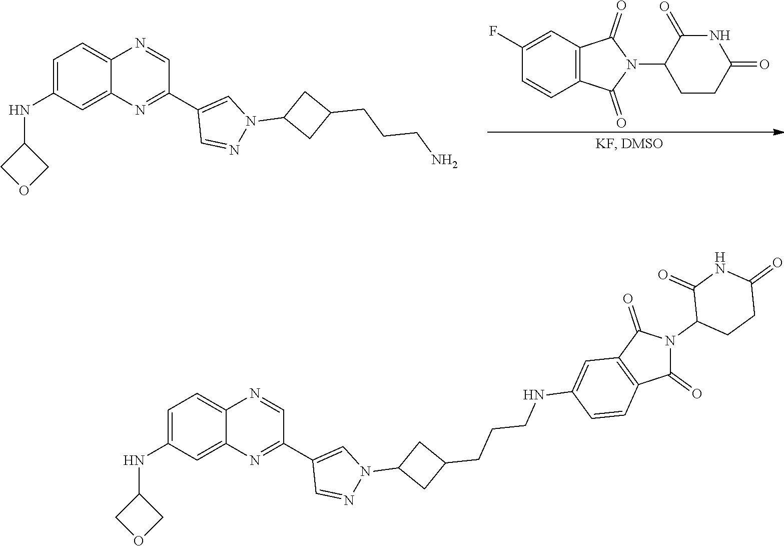 [SMILES: NCCCC1CC(n2cc(-c3cnc4ccc(NC5COC5)cc4n3)cn2)C1 ; O=C1CCC(N2C(=O)c3ccc(F)cc3C2=O)C(=O)N1 ; O=C1CCC(N2C(=O)c3ccc(NCCCC4CC(n5cc(-c6cnc7ccc(NC8COC8)cc7n6)cn5)C4)cc3C2=O)C(=O)N1]

To a solution of 3-(1-(3-(3-aminopropyl)cyclobutyl)-1H-pyrazol-4-yl)-N-(oxetan-3-yl)quinoxalin-6-amine (20 mg, 0.05 mmol) in DMSO (1 mL) were added 2-(2,6-dioxopiperidin-3-yl)-5-fluoroisoindoline-1,3-dione (27 mg, 0.1 mmol) and KF (9 mg, 0.15 mmol). The mixture was stirred at 135° C. for 15 min, before it was purified by reverse phase chromatography (MeOH:H₂O=1:1) to provide the title compound (3 mg, yield: 9%) as yellow solid. MS (ESI) m/z=635.5 [M+H]⁺.

### Example 526. 5-((3-((trans)-3-(3-Cyclopropyl-4-(quinoxalin-2-yl)-1H-pyrazol-1-yl)cyclobutyl)propyl)amino)-2-(2,6-dioxopiperidin-3-yl)isoindoline-1,3-dione (GS-568)

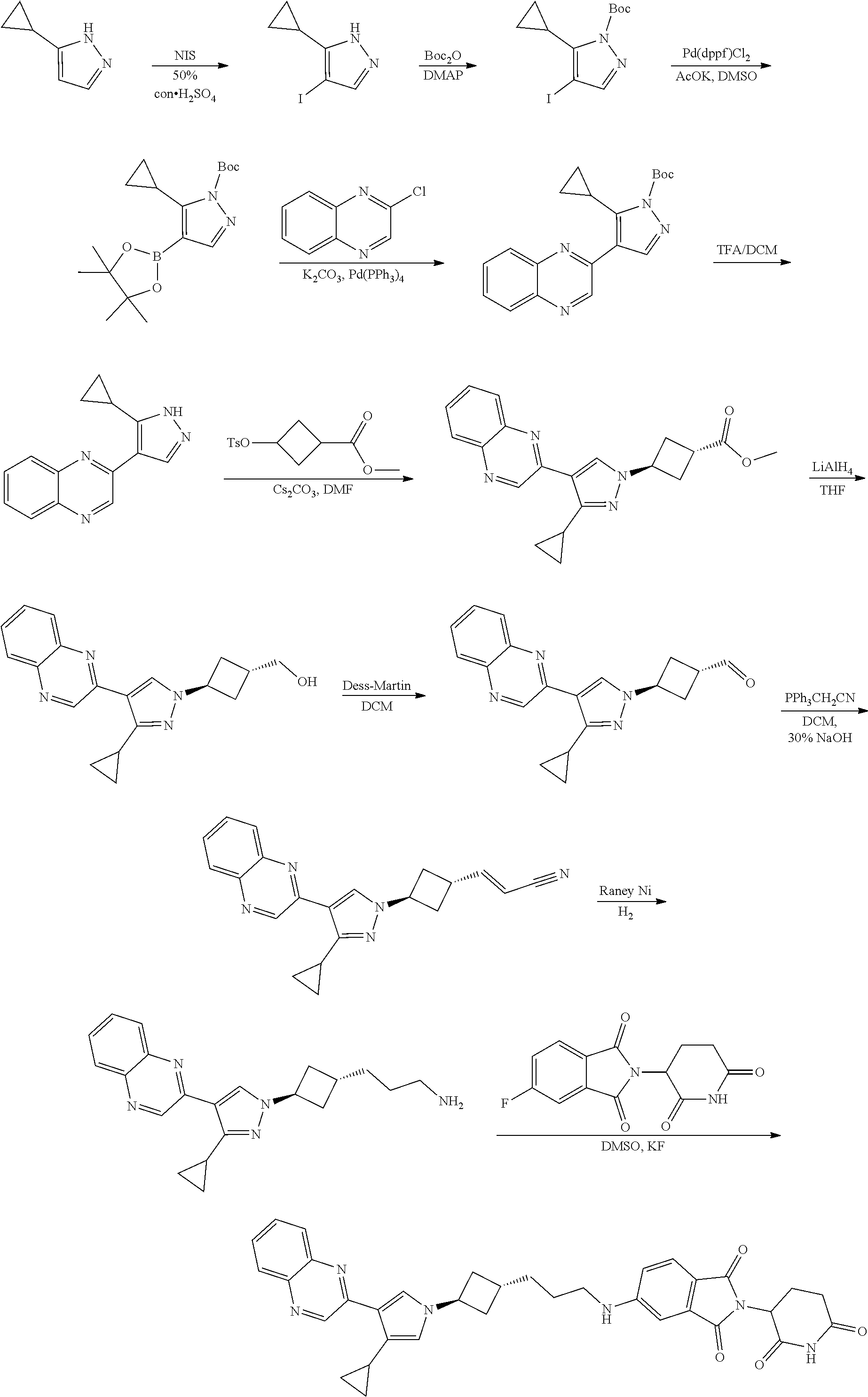

Step 1. Synthesis of 5-cyclopropyl-4-iodo-1H-pyrazole

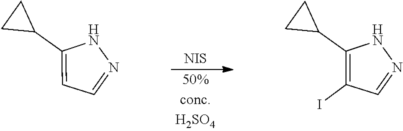 [SMILES: c1cc(C2CC2)[nH]n1 ; "NIS" ; Ic1cn[nH]c1C1CC1]

To a solution of 5-cyclopropyl-1H-pyrazole (2.0 g, 18.49 mmol) in H₂SO₄ solution (50%, 30 mL) was added 1-iodopyrrolidine-2,5-dione (5.04 g, 22.40 mmol) at 0° C. After the reaction mixture was stirred at rt for 3 h, it was poured to water (100 mL), and extracted with EtOAc (100 mL×2). The combine organic layers were washed with sat. NaHCO₃ solution (50 mL) and brine (100 mL), dried and concentrated to provide the title compound (3.5 g, yield: 81%) as light-yellow solid.

Step 2. Synthesis of tert-butyl 3-cyclopropyl-4-iodo-pyrazole-1-carboxylate

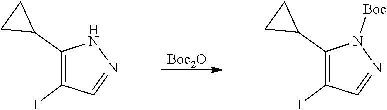 [SMILES: "Boc2O" ; Ic1cn[nH]c1C1CC1 ; CC(C)(C)OC(=O)n1ncc(I)c1C1CC1]

To a solution of 5-cyclopropyl-4-iodo-1H-pyrazole (2 g, 8.55 mmol) in DCM (20 mL) were added Boc₂O (1.87 g, 8.55 mmol) and DMAP (100 mg, 818.55 μmol) at rt. The mixture was stirred at rt for 1 h, and then concentrated. The resulting residue was purified by silica gel chromatography to provide the title compound (2.6 g, yield: 87%) as white solid. MS (ESI) m/z=235.0 [M+H-Boc]⁺.

Step 3. Synthesis of tert-butyl 3-cyclopropyl-4-(4,4,5,5-tetramethyl-1,3,2-dioxaborolan-2-yl)pyrazole-1-carboxylate

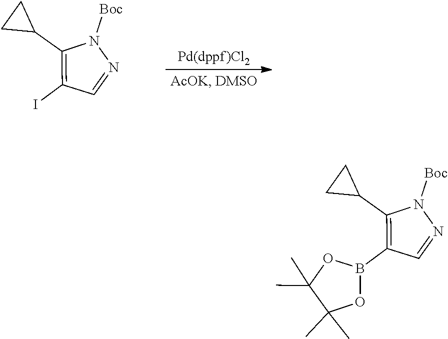 [SMILES: CC(C)(C)OC(=O)n1ncc(I)c1C1CC1 ; "Pd(dppf)Cl2" ; CC(C)(C)OC(=O)n1ncc(B2OC(C)(C)C(C)(C)O2)c1C1CC1]

A mixture of tert-butyl 3-cyclopropyl-4-iodo-pyrazole-1-carboxylate (10 g, 29.93 mmol), 4,4,5,5-tetramethyl-2-(4,4,5,5-tetramethyl-1,3,2-dioxaborolan-2-yl)-1,3,2-dioxaborolane (9.11 g, 35.87 mmol), potassium acetate (6.31 g, 64.25 mmol) and Pd(dppf)Cl₂ (1 g, 29.93 mmol) in DMSO (80 mL) was heated at 80° C. for 2 h. After the mixture was cooled to rt, it was purified by reverse phase chromatography to provide the title compound (5.2 g, yield: 49%) as white solid. MS (ESI) m/z=278.9 [M+H−tBu]⁺.

Step 4 to 5. Synthesis of 2-(3-cyclopropyl-1H-pyrazol-4-yl)quinoxaline

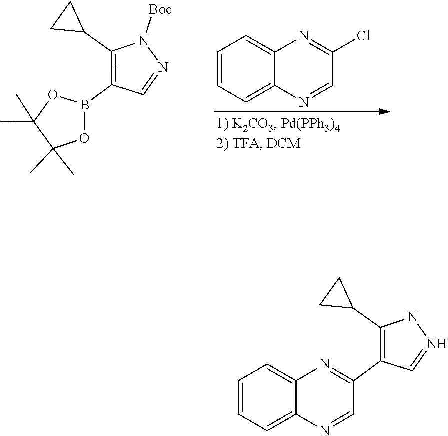 [SMILES: CC(C)(C)OC(=O)n1ncc(B2OC(C)(C)C(C)(C)O2)c1C1CC1 ; Clc1cnc2ccccc2n1 ; c1ccc2nc(-c3cn[nH]c3C3CC3)cnc2c1]

A mixture of 2-chloroquinoxaline (1.52 g, 9.20 mmol), tert-butyl 3-cyclopropyl-4-(4,4,5,5-tetramethyl-1,3,2-dioxaborolan-2-yl)pyrazole-1-carboxylate (3.6 g, 10.13 mmol) and Pd(PPh)₃ (150 mg, 9.20 mmol) in toluene (30 mL), ethanol (15 mL) and water (7.5 mL) was heated at 90° C. for 1 h under N₂. The mixture was concentrated. The resulting residue was dissolved in EtOAc (150 mL), washed with brine (100 mL). The organic layer was dried and concentrated. The resulting residue was dissolved in DCM (30 mL). TFA (10 mL) was added at rt. The mixture was stirred at rt for 1 h, before it was concentrated. The resulting residue was dissolved in EtOAc (100 mL), washed with sat. Na₂CO₃ solution (30 mL) and brine (50 mL). The organic phase was dried and concentrated. The resulting residue was purified by silica gel chromatography to provide the title compound (800 mg, yield: 34%) as brown solid.

Step 6. Synthesis of methyl trans-3-(3-cyclopropyl-4-(quinoxalin-2-yl)-1H-pyrazol-1-yl)cyclobutane-1-carboxylate

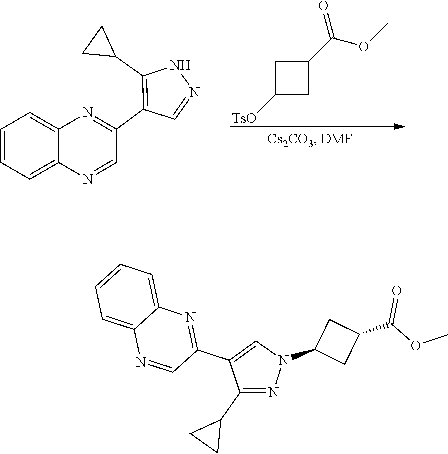 [SMILES: c1ccc2nc(-c3cn[nH]c3C3CC3)cnc2c1 ; COC(=O)C1CC(OS(=O)(=O)c2ccc(C)cc2)C1 ; COC(=O)[C@H]1C[C@H](n2cc(-c3cnc4ccccc4n3)c(C3CC3)n2)C1]

A mixture of 2-(3-cyclopropyl-1H-pyrazol-4-yl)quinoxaline (800 mg, 3.39 mmol) and methyl 3-(p-tolylsulfonyloxy)cyclobutanecarboxylate (see step 1 of GS-579 for synthesis, 1.25 g, 4.40 mmol) in DMF (5 mL) was heated at 90° C. for 1 h. The mixture was cooled to rt, poured to water (50 mL), and extracted with EtOAc (50 mL×2). The combined organic layers were washed with brine (60 mL), dried and concentrated. The resulting residue was purified by prep-TLC to provide the title compound (490 mg, yield: 39%) as light brown solid. MS (ESI) m/z=349.3 [M+H]⁺.

Step 7. Synthesis of (trans-3-(3-cyclopropyl-4-(quinoxalin-2-yl)-1H-pyrazol-1-yl)cyclobutyl)methanol

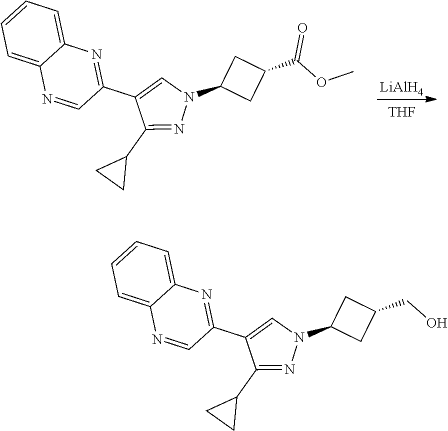

To a solution of methyl trans-3-(3-cyclopropyl-4-(quinoxalin-2-yl)-1H-pyrazol-1-yl)cyclobutane-1-carboxylate (490 mg, 1.27 mmol) in THF (15 mL) was added LiAlH₄ (100 mg, 2.63 mmol) at 0° C. The reaction mixture was stirred for 30 min. The mixture was quenched with water, filtered, and concentrated to provide the title compound (320 mg, yield: 77%) as light brown oil. MS (ESI) m/z=321.3 [M+H]⁺.

Step 8. Synthesis of trans-3-(3-cyclopropyl-4-(quinoxalin-2-yl)-1H-pyrazol-1-yl)cyclobutane-1-carbaldehyde

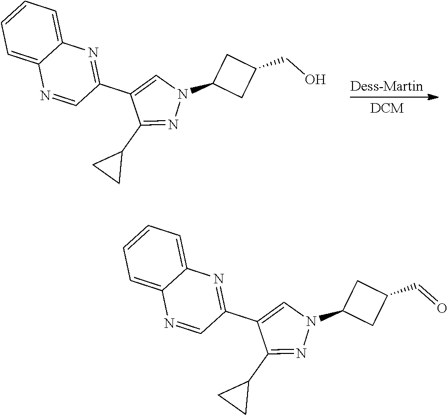 [SMILES: OC[C@H]1C[C@H](n2cc(-c3cnc4ccccc4n3)c(C3CC3)n2)C1 ; O=C[C@H]1C[C@H](n2cc(-c3cnc4ccccc4n3)c(C3CC3)n2)C1]

To a solution of (trans-3-(3-cyclopropyl-4-(quinoxalin-2-yl)-1H-pyrazol-1-yl)cyclobutyl)methanol (320 mg, 968.83 μmol) in DCM (15 mL) was added Dess-Martin Periodinane (825.53 mg, 1.95 mmol) at 0° C. The mixture was warmed to rt and stirred for 1 h. The mixture was quenched with NaHSO₃, washed with sat. Na₂CO₃ solution (15 mL) and brine (10 mL). The organic phase was dried and concentrated to provide the title compound (300 mg, yield: 82%) as light-yellow oil. MS (ESI) m/z=319.3 [M+H]⁺.

Step 9. Synthesis of 4-(trans-3-(3-cyclopropyl-4-(quinoxalin-2-yl)-1H-pyrazol-1-yl)cyclobutyl)but-2-enenitrile

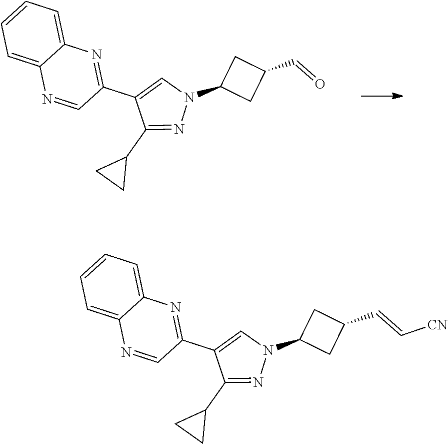 [SMILES: O=C[C@H]1C[C@H](n2cc(-c3cnc4ccccc4n3)c(C3CC3)n2)C1 ; N#C/C=C/[C@H]1C[C@H](n2cc(-c3cnc4ccccc4n3)c(C3CC3)n2)C1]

To a solution of trans-3-(3-cyclopropyl-4-(quinoxalin-2-yl)-1H-pyrazol-1-yl)cyclobutane-1-carbaldehyde (320 mg, 874.45 μmol), (cyanomethyl)triphenylphosphonium chloride (360 mg, 1.07 mmol) in DCM (8 mL) was added NaOH (30%, 0.1 mL) at rt. The mixture was stirred for 1 h, before it was diluted with DCM (50 mL). The mixture was washed with brine (20 mL), and dried over Na₂SO₄. The organic phase was concentrated. The resulting residue was purified by reverse phase chromatography to provide the title compound (320 mg, yield: 96%) as light brown solid. MS (ESI) m/z=342.3 [M+H]⁺.

Step 10. Synthesis of 3-(trans-3-(3-cyclopropyl-4-(quinoxalin-2-yl)-1H-pyrazol-1-yl)cyclobutyl)propan-1-amine

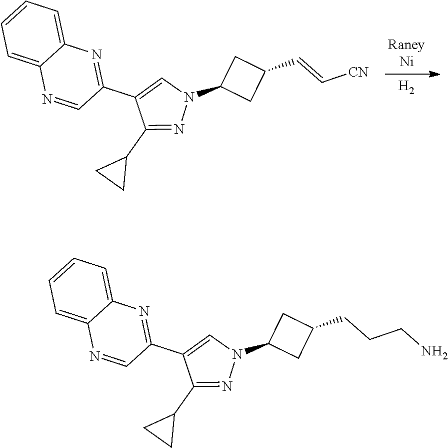 [SMILES: N#C/C=C/[C@H]1C[C@H](n2cc(-c3cnc4ccccc4n3)c(C3CC3)n2)C1 ; "H2" ; NCCC[C@H]1C[C@H](n2cc(-c3cnc4ccccc4n3)c(C3CC3)n2)C1]

To a solution of 4-(trans-3-(3-cyclopropyl-4-(quinoxalin-2-yl)-1H-pyrazol-1-yl)cyclobutyl)but-2-enenitrile (320 mg, 843.56 μmol) in THF (8 mL) and ammonia (7 M in methanol, 8 mL) was added Raney Ni (1 g, 17.04 mmol) at rt. The reaction mixture was stirred under H₂ for 2 h. The mixture was filtered, and the filtrate was concentrated to provide the title compound (280 mg, yield: 74%) as brown oil. MS (ESI) m/z=348.3 [M+H]⁺.

Step 11 Synthesis of 5-((3-(trans-3-(3-cyclopropyl-4-(quinoxalin-2-yl)-1H-pyrazol-1-yl)cyclobutyl)propyl)amino)-2-(2,6-dioxopiperidin-3-yl)isoindoline-1,3-dione

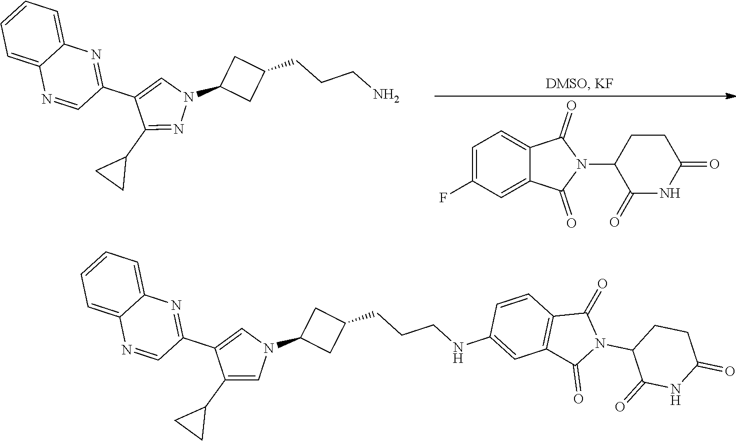 [SMILES: NCCC[C@H]1C[C@H](n2cc(-c3cnc4ccccc4n3)c(C3CC3)n2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccccc7n6)c(C6CC6)c5)C4)cc3C2=O)C(=O)N1]

A mixture of 3-(trans-3-(3-cyclopropyl-4-(quinoxalin-2-yl)-1H-pyrazol-1-yl)cyclobutyl)propan-1-amine (20 mg, 32.09 μmol), 2-(2,6-dioxo-3-piperidyl)-5-fluoro-isoindoline-1,3-dione (20 mg, 72.41 μmol) and KF (6 mg, 103.28 μmol) in DMSO (2 mL) was irradiated at 125° C. in the microwave reactor for 20 min. After the mixture was cooled to rt, it was purified by reverse phase chromatography to provide the title compound (10 mg, yield: 52%) as yellow solid. MS (ESI) m/z=604.5 [M+H]⁺.

### Example 527. 5-((6-(3-Cyclopropyl-4-(7-morpholinoquinoxalin-2-yl)-1H-pyrazol-1-yl)hexyl)amino)-2-(2,6-dioxopiperidin-3-yl)isoindoline-1,3-dione (GS-569)

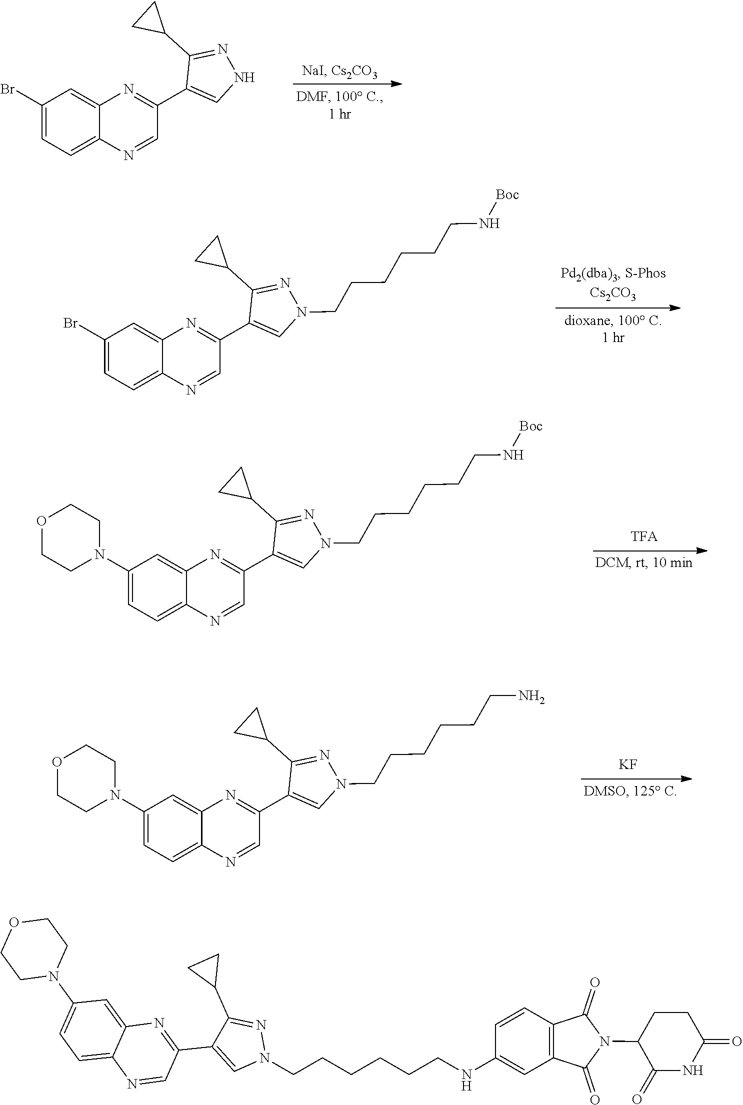 [SMILES: Brc1ccc2ncc(-c3c[nH]nc3C3CC3)nc2c1 ; CC(C)(C)OC(=O)NCCCCCCn1cc(-c2cnc3ccc(Br)cc3n2)c(C2CC2)n1 ; CC(C)(C)OC(=O)NCCCCCCn1cc(-c2cnc3ccc(N4CCOCC4)cc3n2)c(C2CC2)n1 ; NCCCCCCn1cc(-c2cnc3ccc(N4CCOCC4)cc3n2)c(C2CC2)n1 ; "KF" ; O=C1CCC(N2C(=O)c3ccc(NCCCCCCn4cc(-c5cnc6ccc(N7CCOCC7)cc6n5)c(C5CC5)n4)cc3C2=O)C(=O)N1]

GS-569 was synthesized following the similar procedures as described for GS-529 (24.3 mg) as brown solid. MS (ESI) m/z=677.6 [M+H]⁺.

### Example 528. 5-((6-(3-Cyclopropyl-4-(7-(4-methylpiperazin-1-yl)quinoxalin-2-yl)-1H-pyrazol-1-yl)hexyl)amino)-2-(2,6-dioxopiperidin-3-yl)isoindoline-1,3-dione (GS-570)

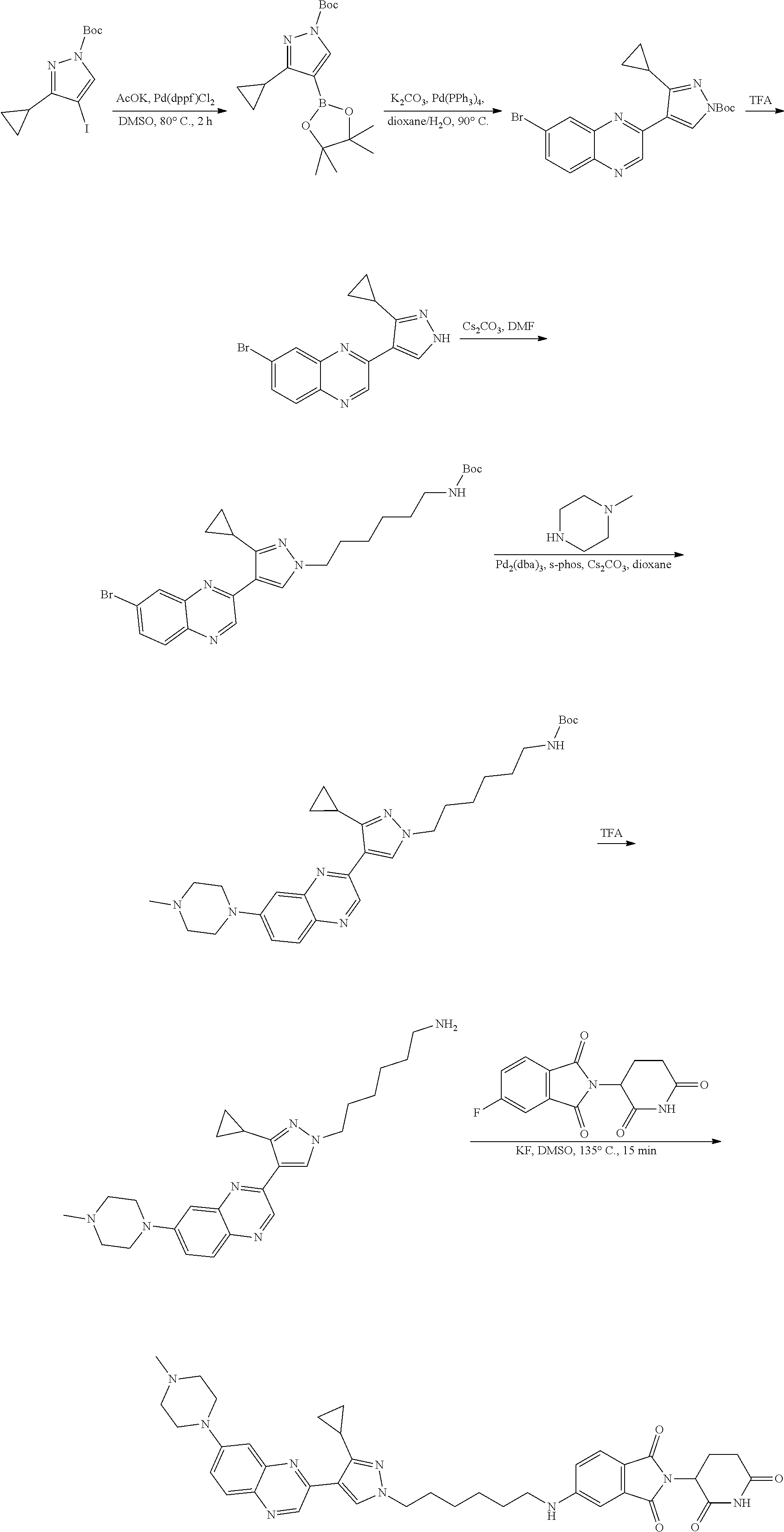

Step 1 to 4. Synthesis of tert-butyl (6-(4-(7-bromoquinoxalin-2-yl)-3-cyclopropyl-1H-pyrazol-1-yl)hexyl)carbamate

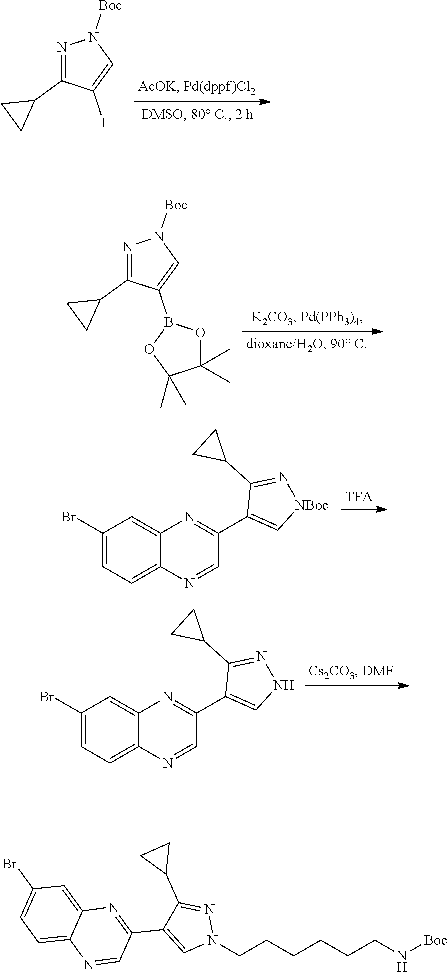

The title compound was synthesized following the similar procedures as described for steps 3 to 6 of GS-568 (1.4 g) as yellow oil. MS (ESI) m/z=514.3 [M+H]⁺.

Step 5 to 7. Synthesis of 5-((6-(3-cyclopropyl-4-(7-(4-methylpiperazin-1-yl)quinoxalin-2-yl)-1H-pyrazol-1-yl)hexyl)amino)-2-(2,6-dioxopiperidin-3-yl)isoindoline-1,3-dione

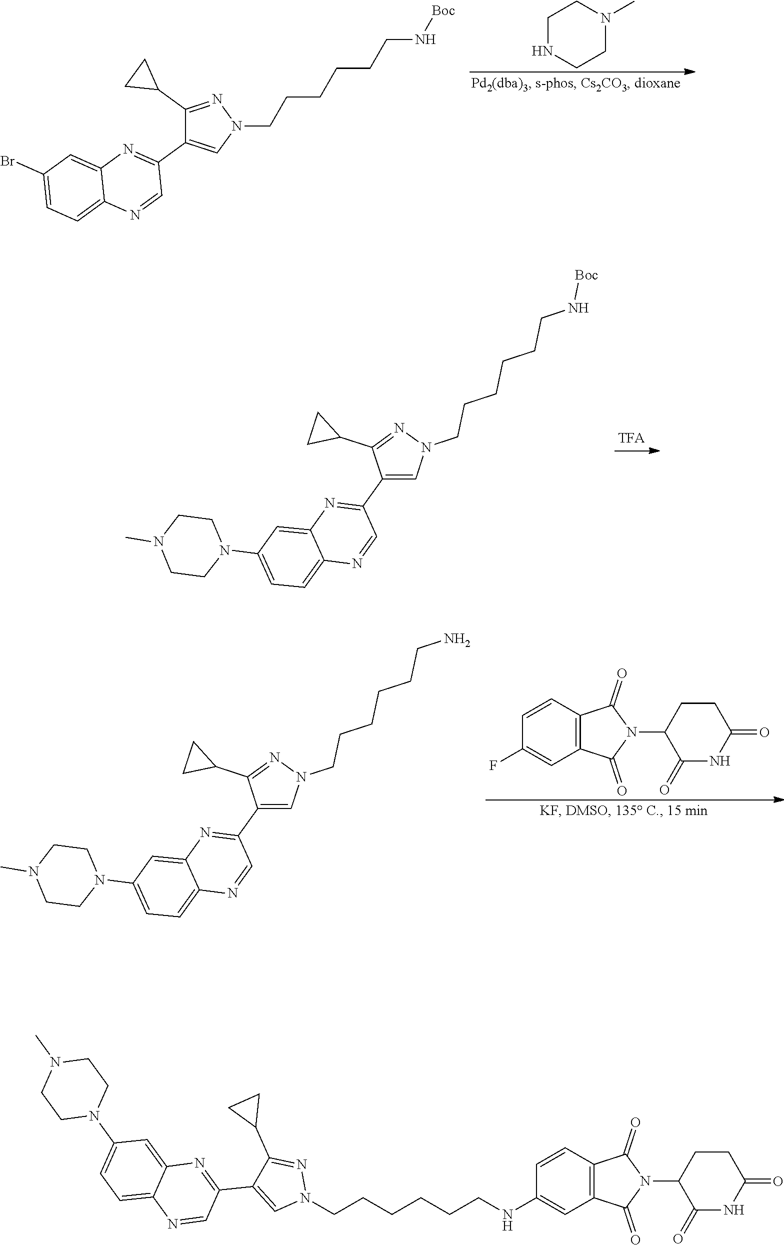

GS-570 was synthesized following the similar procedures as described for steps 3 to 5 of GS-529 (13 mg) as yellow solid. MS (ESI) m/z=690.7 [M+H]⁺.

### Example 529. 5-((6-(3-Cyclopropyl-4-(7-(tetrahydro-2H-pyran-4-yl)quinoxalin-2-yl)-1H-pyrazol-1-yl)hexyl)amino)-2-(2,6-dioxopiperidin-3-yl)isoindoline-1,3-dione (GS-571)

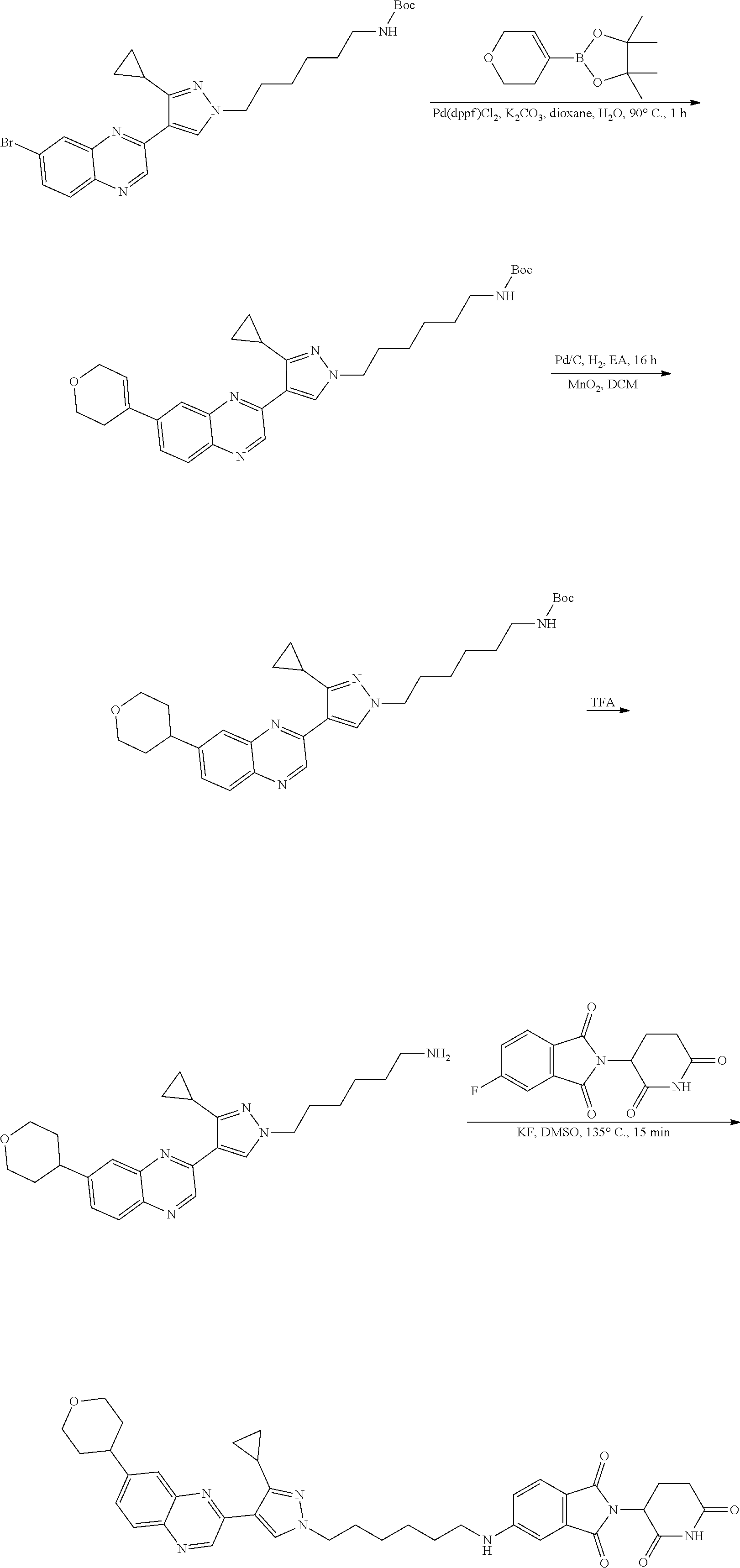

GS-571 was synthesized following the similar procedures as described for GS-541 (4 mg) as yellow solid. MS (ESI) m/z=676.7 [M+H]⁺.

### Example 530. 5-((6-(3-Cyclopropyl-4-(7-(1-methylpiperidin-4-yl)quinoxalin-2-yl)-1H-pyrazol-1-yl)hexyl)amino)-2-(2,6-dioxopiperidin-3-yl)isoindoline-1,3-dione (GS-572)

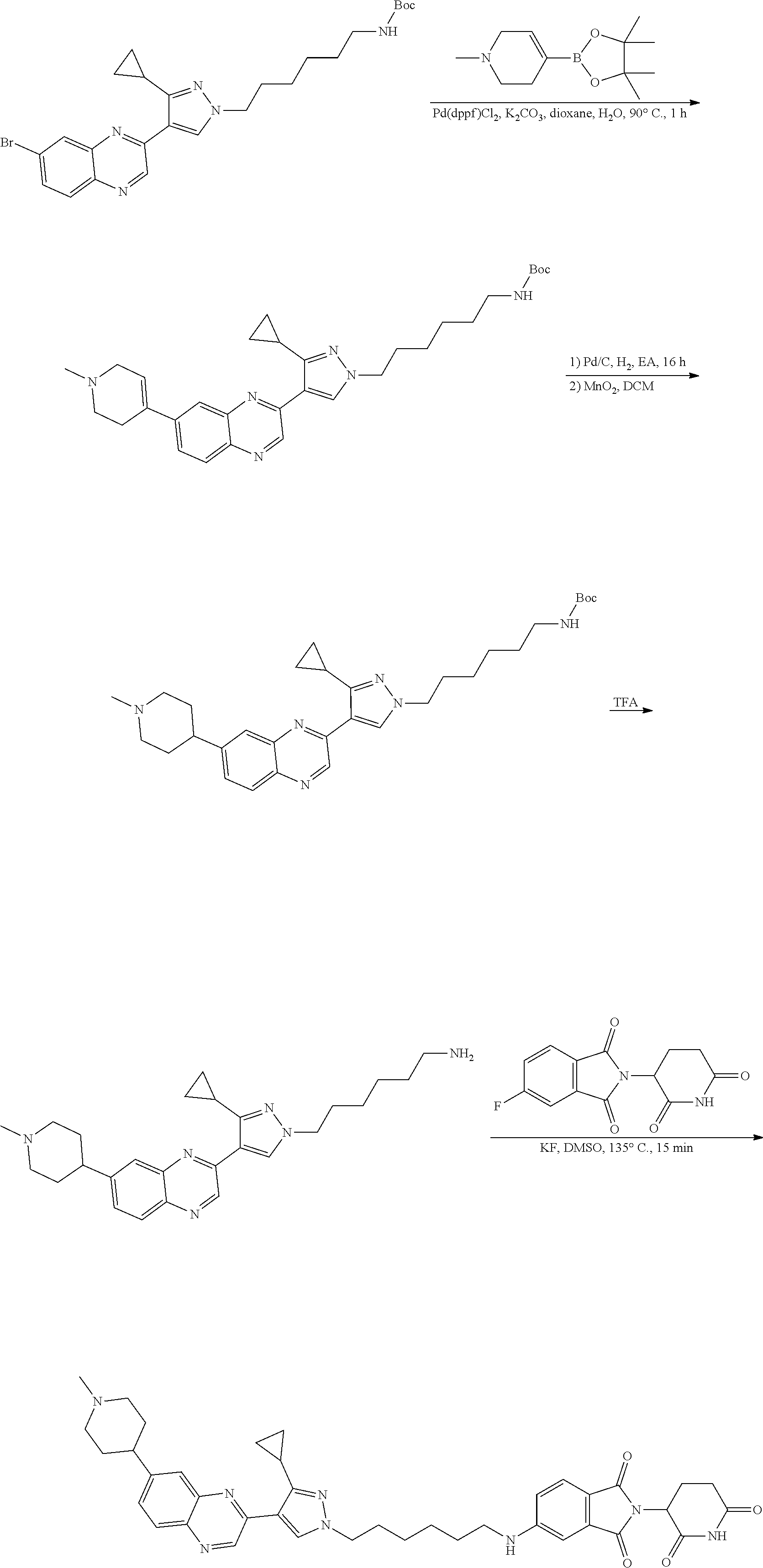

GS-572 was synthesized following the similar procedures as described for GS-541 (4 mg) as yellow solid. MS (ESI) m/z=676.7 [M+H]⁺.

### Example 531. 5-((3-(trans-3-(3-Cyclopropyl-4-(6-morpholinoquinoxalin-2-yl)-1H-pyrazol-1-yl)cyclobutyl)propyl)amino)-2-(2,6-dioxopiperidin-3-yl)isoindoline-1,3-dione (GS-573)

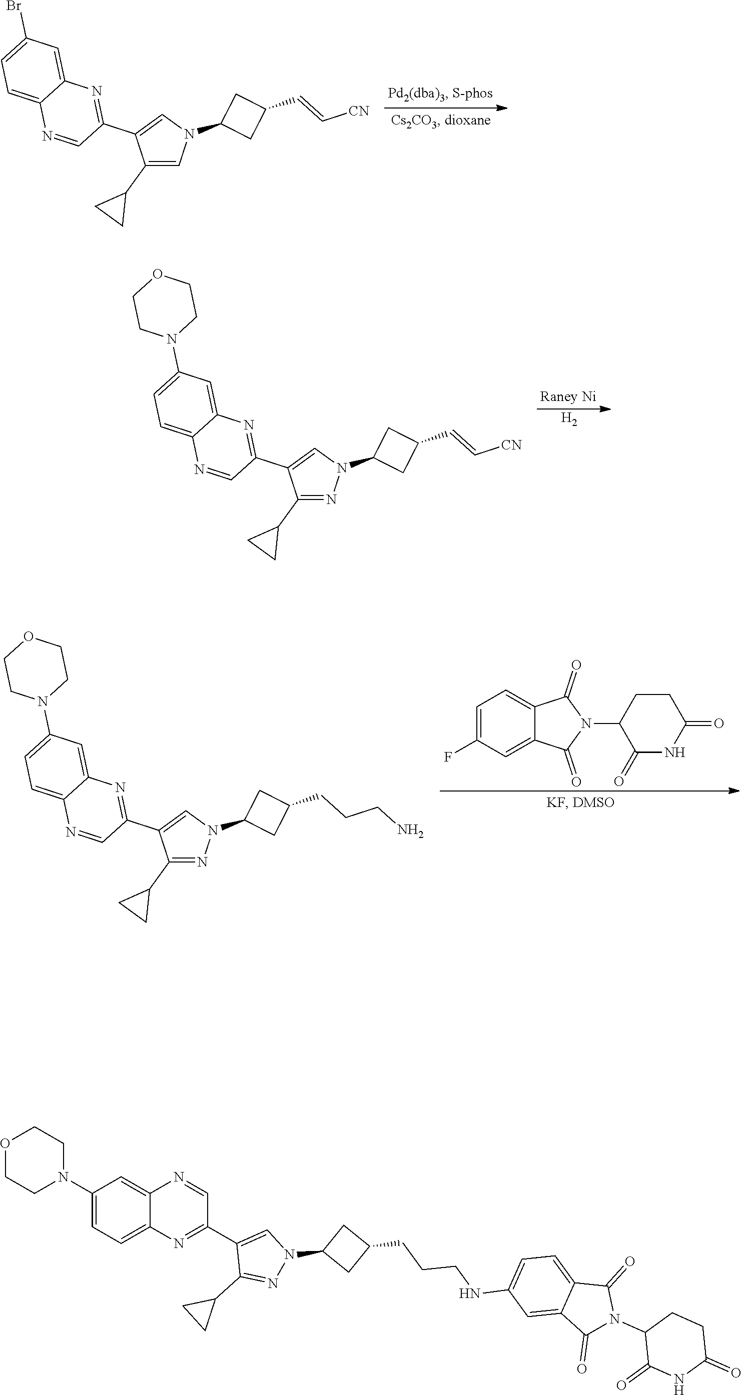 [SMILES: N#C/C=C/[C@H]1C[C@H](n2cc(-c3cnc4ccc(Br)cc4n3)c(C3CC3)c2)C1 ; N#C/C=C/[C@H]1C[C@H](n2cc(-c3cnc4ccc(N5CCOCC5)cc4n3)c(C3CC3)n2)C1 ; "H2" ; NCCC[C@H]1C[C@H](n2cc(-c3cnc4ccc(N5CCOCC5)cc4n3)c(C3CC3)n2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7cc(N8CCOCC8)ccc7n6)c(C6CC6)n5)C4)cc3C2=O)C(=O)N1]

GS-573 was synthesized following the similar procedures as described for GS-585 (3 mg) as yellow solid. MS (ESI) m/z=689.7 [M+H]⁺.

### Example 532. 5-((3-(trans-3-(3-Cyclopropyl-4-(6-((1R,4R)-5-methyl-2,5-diazabicyclo[2.2.1]heptan-2-yl)quinoxalin-2-yl)-1H-pyrazol-1-yl)cyclobutyl)propyl)amino)-2-(2,6-dioxopiperidin-3-yl)isoindoline-1,3-dione (GS-574)

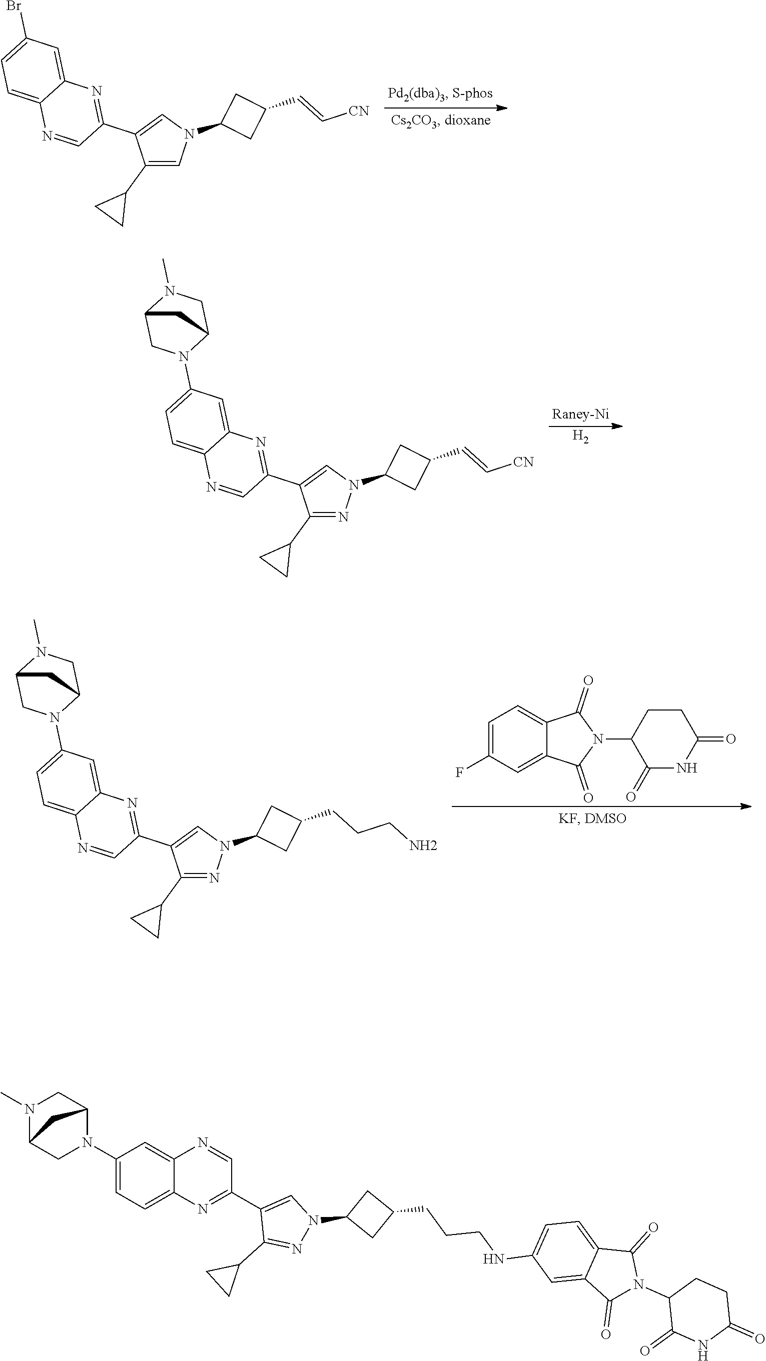 [SMILES: N#C/C=C/[C@H]1C[C@H](n2cc(-c3cnc4ccc(Br)cc4n3)c(C3CC3)c2)C1 ; CN1C[C@H]2C[C@@H]1CN2c1ccc2ncc(-c3cn([C@H]4C[C@H](/C=C/C#N)C4)nc3C3CC3)nc2c1 ; "H2" ; CN1C[C@H]2C[C@@H]1CN2c1ccc2ncc(-c3cn([C@H]4C[C@H](CCCN)C4)nc3C3CC3)nc2c1 ; O=C1CCC(N2C(=O)c3ccc(F)cc3C2=O)C(=O)N1 ; CN1C[C@H]2C[C@@H]1CN2c1ccc2nc(-c3cn([C@H]4C[C@H](CCCNc5ccc6c(c5)C(=O)N(C5CCC(=O)NC5=O)C6=O)C4)nc3C3CC3)cnc2c1]

GS-574 was synthesized following the similar procedures as described for GS-585 (6 mg) as red solid. MS (ESI) m/z=714.7 [M+H]⁺.

### Example 533. 5-((3-(trans-3-(4-(7-((S)-3,4-Dimethylpiperazin-1-yl)quinoxalin-2-yl)-1H-pyrazol-1-yl)cyclobutyl)propyl)amino)-2-(2,6-dioxopiperidin-3-yl)isoindoline-1,3-dione (GS-575)

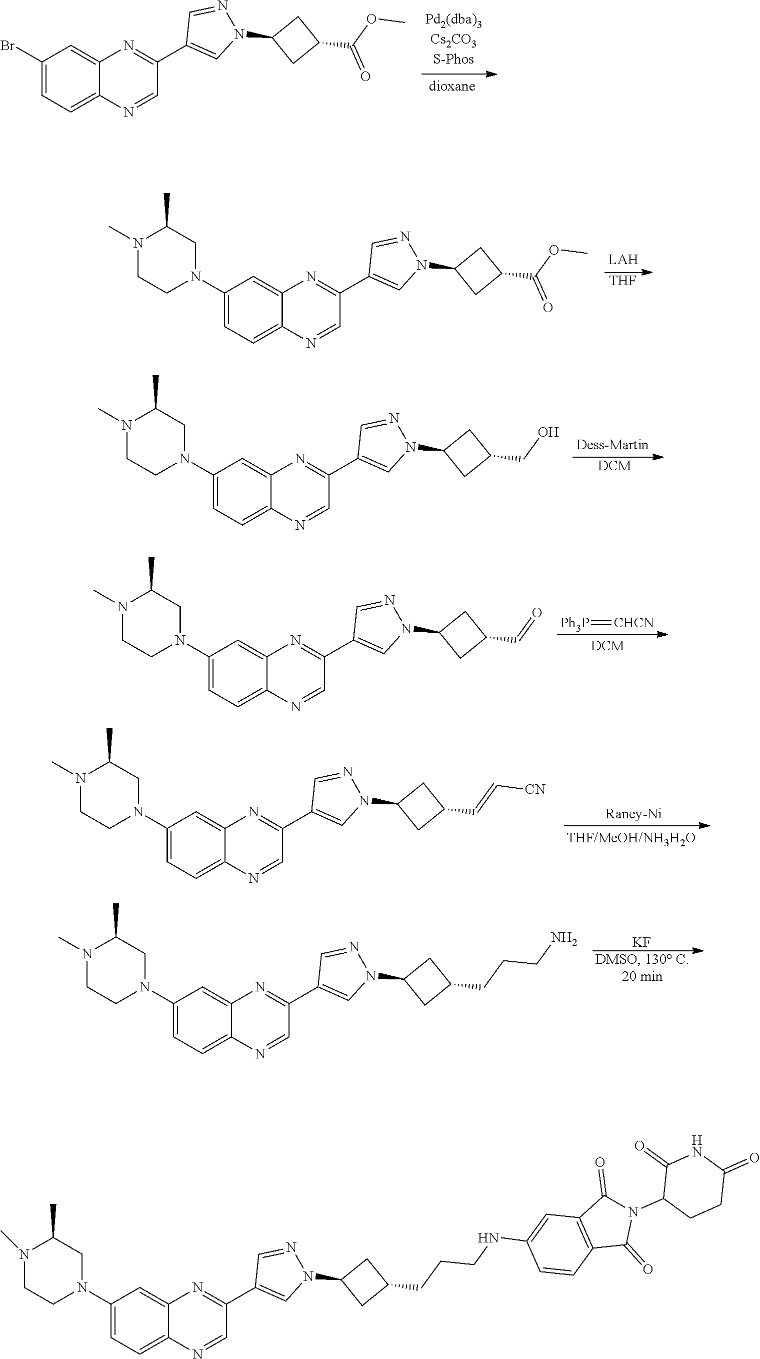

GS-575 was synthesized following the similar procedures as described for GS-399 (37.6 mg) as yellow solid. MS (ESI) m/z=676.6 [M+H]⁺.

### Example 534. 2-(2,6-Dioxopiperidin-3-yl)-5-((3-(trans-3-(4-(7-((S)-3-methylpiperazin-1-yl)quinoxalin-2-yl)-1H-pyrazol-1-yl)cyclobutyl)propyl)amino)isoindoline-1,3-dione (GS-576)

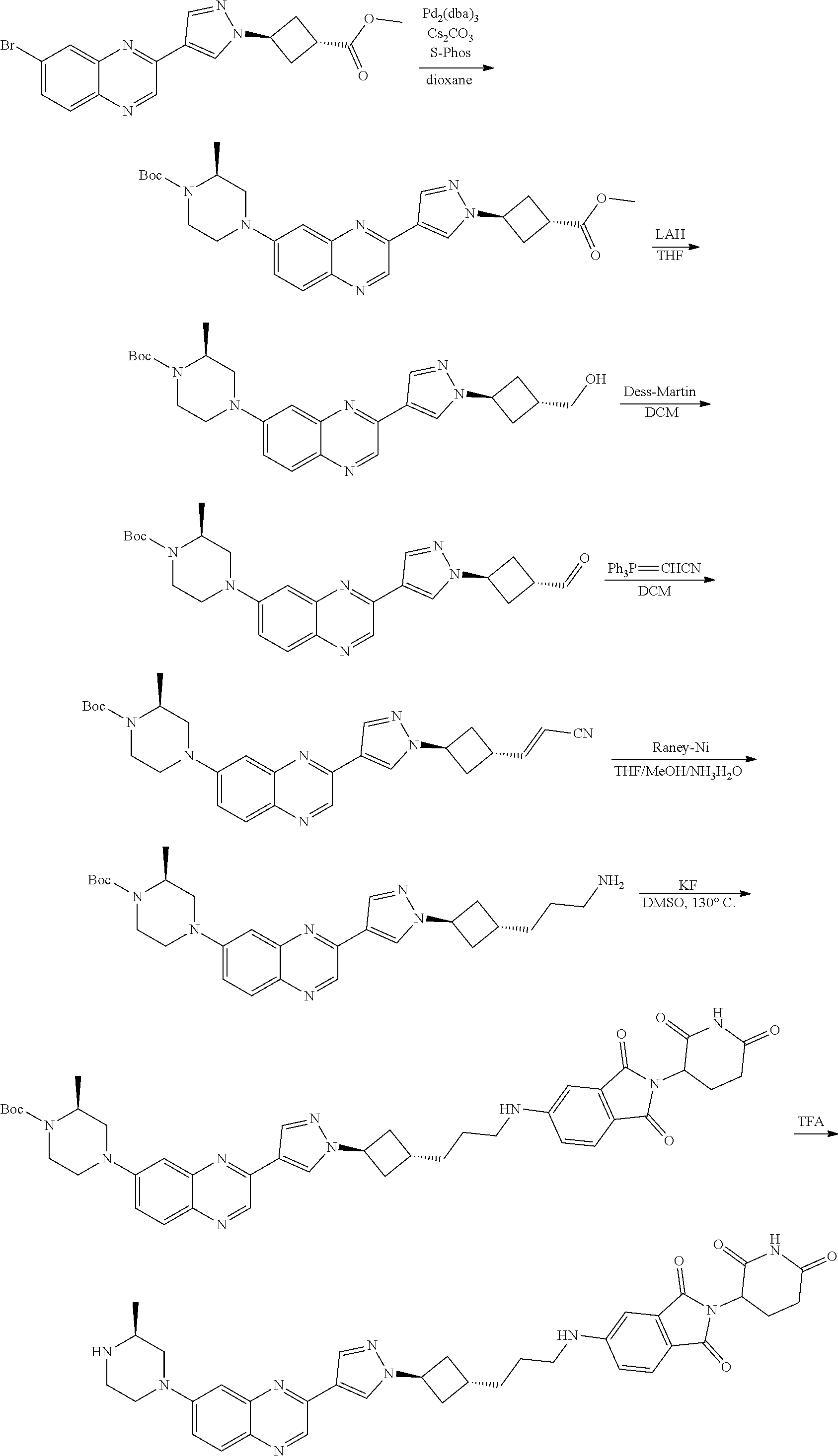

Steps 1 to 6. Synthesis of tert-butyl (2S)-4-(3-(1-(trans-3-(3-((2-(2,6-dioxopiperidin-3-yl)-1,3-dioxoisoindolin-5-yl)amino)propyl)cyclobutyl)-1H-pyrazol-4-yl)quinoxalin-6-yl)-2-methylpiperazine-1-carboxylate

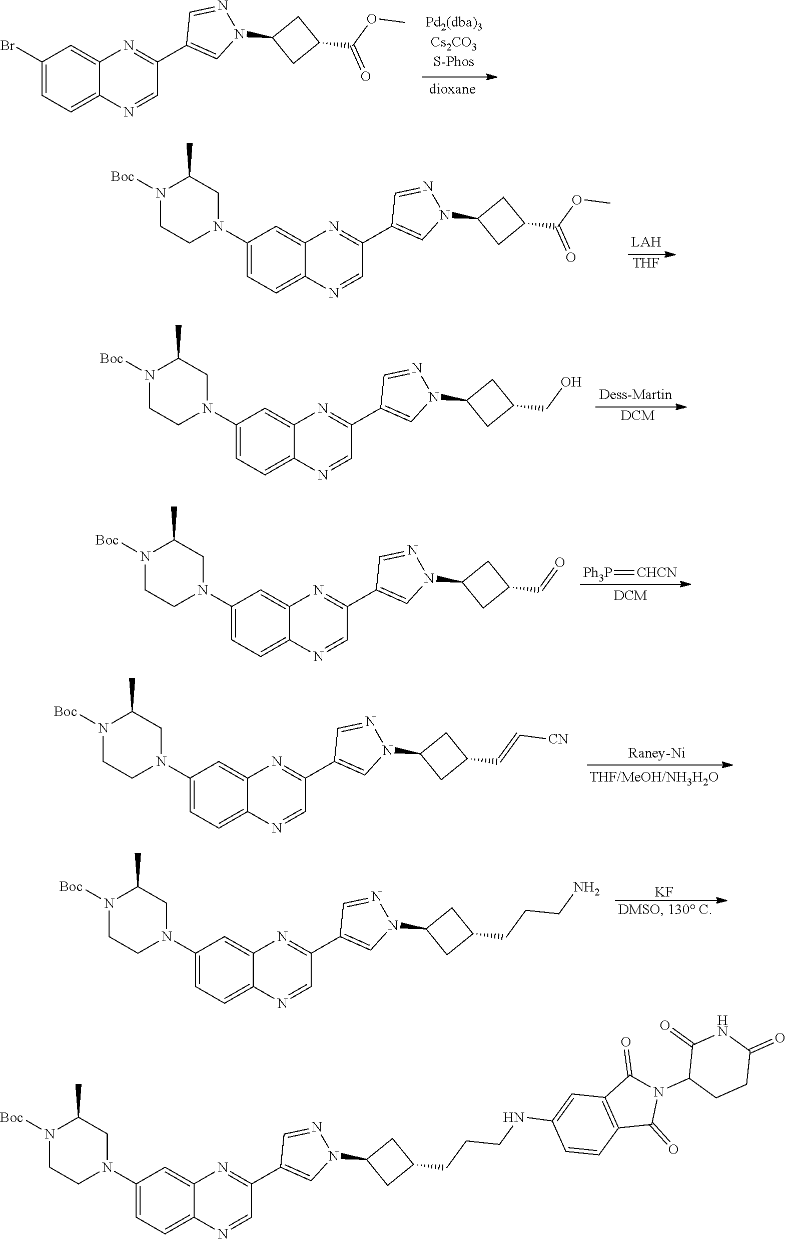

The title compound was synthesized following the similar procedures as described for GS-399 (45 mg) as yellow solid. MS (ESI) m/z=762.8 [M+H]⁺.

Step 7. Synthesis of 2-(2,6-dioxopiperidin-3-yl)-5-((3-(trans-3-(4-(7-((S)-3-methylpiperazin-1-yl)quinoxalin-2-yl)-1H-pyrazol-1-yl)cyclobutyl)propyl)amino)isoindoline-1,3-dione

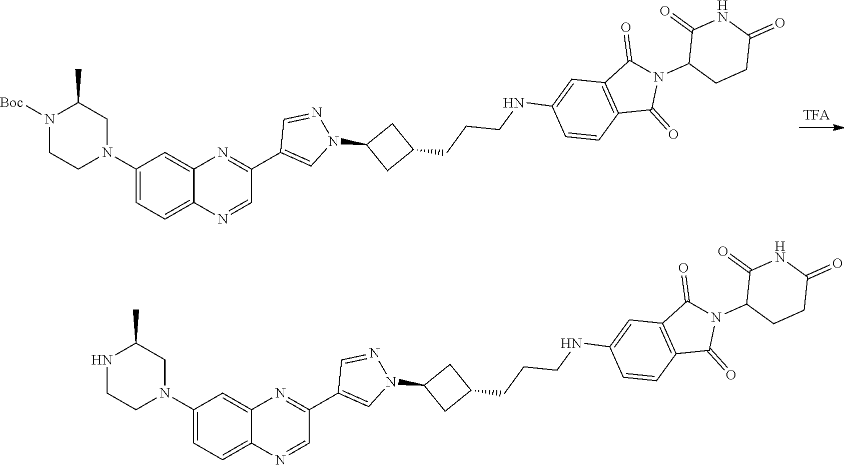 [SMILES: C[C@H]1CN(c2ccc3ncc(-c4cnn([C@H]5C[C@H](CCCNc6ccc7c(c6)C(=O)N(C6CCC(=O)NC6=O)C7=O)C5)c4)nc3c2)CCN1C(=O)OC(C)(C)C ; C[C@H]1CN(c2ccc3ncc(-c4cnn([C@H]5C[C@H](CCCNc6ccc7c(c6)C(=O)N(C6CCC(=O)NC6=O)C7=O)C5)c4)nc3c2)CCN1]

To a stirred solution of tert-butyl (2S)-4-(3-(1-(trans-3-(3-((2-(2,6-dioxopiperidin-3-yl)-1,3-dioxoisoindolin-5-yl)amino)propyl)cyclobutyl)-1H-pyrazol-4-yl)quinoxalin-6-yl)-2-methylpiperazine-1-carboxylate (45 mg, 59 μmol) in DCM (2 mL) was added TFA (1 mL). The reaction mixture was stirred at rt for 10 min, before it was concentrated. The resulting residue was purified by reverse phase chromatography (0-70% MeCN in H₂O) to provide the title compound (27 mg, yield: 69%) as red solid. MS (ESI) m/z=662.5 [M+H]⁺.

### Example 535. 2-(2,6-Dioxopiperidin-3-yl)-5-(((trans-3-(4-(pyridin-2-yl)-1H-pyrazol-1-yl)cyclobutyl)methyl)amino)isoindoline-1,3-dione (GS-577)

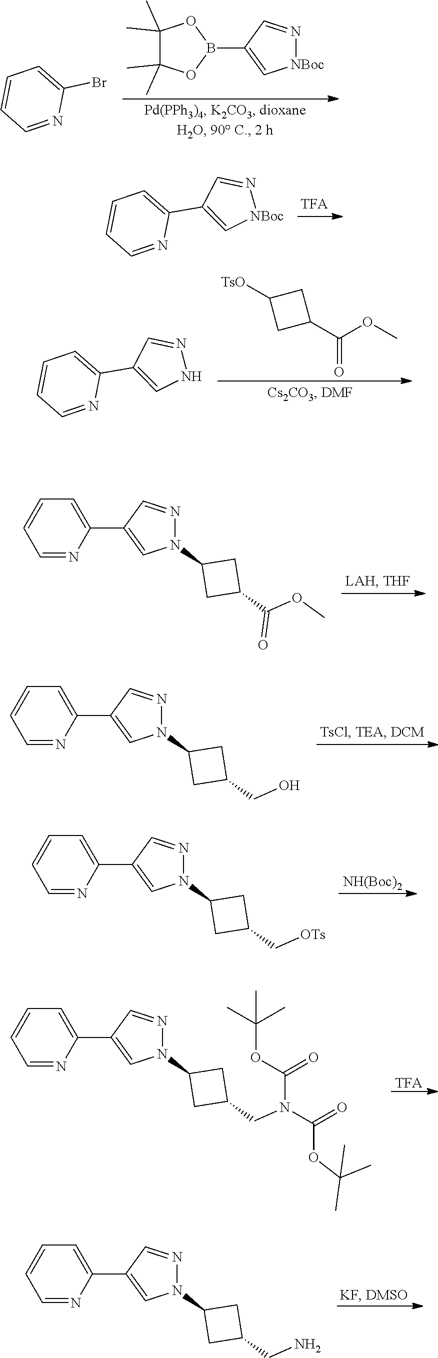

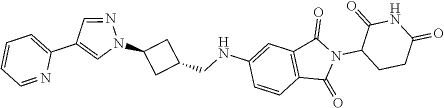 [SMILES: O=C1CCC(N2C(=O)c3ccc(NC[C@H]4C[C@H](n5cc(-c6ccccn6)cn5)C4)cc3C2=O)C(=O)N1]

Step 1. Synthesis of tert-butyl 4-(pyridin-2-yl)-1H-pyrazole-1-carboxylate

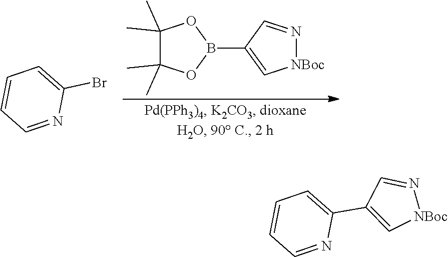

To a solution of 2-bromopyridine (2 g, 12.66 mmol) in dioxane (30 mL) and H₂O (5 mL) were added tert-butyl 4-(4,4,5,5-tetramethyl-1,3,2-dioxaborolan-2-yl)pyrazole-1-carboxylate (3.72 g, 12.66 mmol), Pd(dppf)Cl₂ (0.46 g, 632.93 μmol) and K₂CO₃ (3.5 g, 25.32 mmol). The mixture was stirred at 90° C. for 2 h before it was concentrated. The resulting residue was purified by silica gel chromatography (petroleum ether:EtOAc=2:1) to provide the title compound (2.6 g, yield: 84%) as yellow oil. MS (ESI) m/z=246.4 [M+H]⁺.

Step 2. Synthesis of 2-(1H-pyrazol-4-yl)pyridine

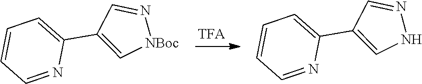 [SMILES: CC(C)(C)OC(=O)n1cc(-c2ccccn2)cn1 ; c1ccc(-c2cn[nH]c2)nc1]

To a solution of tert-butyl 4-(pyridin-2-yl)-1H-pyrazole-1-carboxylate (2.6 g, 10.60 mmol) in DCM (5 mL) was added TFA (2 mL). The mixture was stirred at rt for 10 min, before it was concentrated to provide the title compound (1.5 g, yield: 97%) as yellow oil. MS (ESI) m/z=146.4 [M+H]⁺.

Step 3. Synthesis of methyl trans-3-(4-(pyridin-2-yl)-1H-pyrazol-1-yl)cyclobutane-1-carboxylate

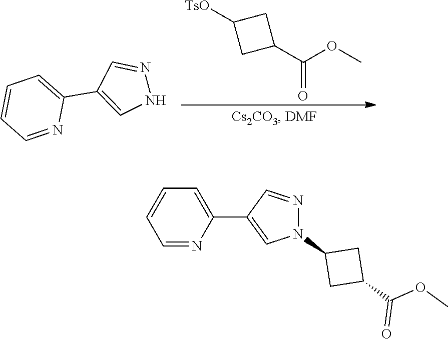 [SMILES: c1ccc(-c2cn[nH]c2)nc1 ; COC(=O)C1CC(OS(=O)(=O)c2ccc(C)cc2)C1 ; COC(=O)[C@H]1C[C@H](n2cc(-c3ccccn3)cn2)C1]

To a solution of 2-(1H-pyrazol-4-yl)pyridine (1.5 g, 10.33 mmol) in DMF (20 mL) were added methyl 3-(p-tolylsulfonyloxy)cyclobutanecarboxylate (see step 1 of GS-579 for synthesis, 2.94 g, 10.33 mmol) and Cs₂CO₃ (6.72 g, 20.67 mmol). The mixture was stirred at 100° C. for 2 h. Water (50 mL) was added. The mixture was extracted with EtOAc (50 mL). The organic phase was concentrated, and the resulting residue was purified by silica gel chromatography (petroleum ether:EtOAc=1:1) to provide the title compound (470 mg, yield: 18%) as yellowish oil. MS (ESI) m/z=258.3 [M+H]⁺.

Step 4. Synthesis of (trans-3-(4-(pyridin-2-yl)-1H-pyrazol-1-yl)cyclobutyl)methanol

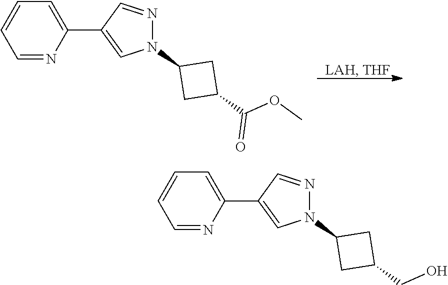

To a solution of methyl trans-3-(4-(pyridin-2-yl)-1H-pyrazol-1-yl)cyclobutane-1-carboxylate (320 mg, 1.24 mmol) in THF (10 mL) was added LAH (94.52 mg, 2.49 mmol). The mixture was stirred at rt for 1 h. Three drops of water were added. The mixture was stirred for another 30 min, before it was filtered. The filtrate was concentrated to provide the title compound (280 mg, yield: 98%) as yellowish oil. MS (ESI) m/z=230.3 [M+H]⁺.

Step 5. Synthesis of (trans-3-(4-(pyridin-2-yl)-1H-pyrazol-1-yl)cyclobutyl)methyl 4-methylbenzenesulfonate

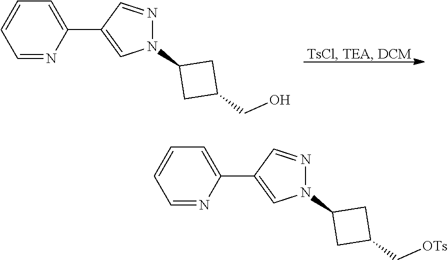

To a solution of (trans-3-(4-(pyridin-2-yl)-1H-pyrazol-1-yl)cyclobutyl)methanol (50 mg, 218.08 μmol) in DCM (10 mL) were added TsCl (23.07 mg, 327.12 μmol), DMAP (2.66 mg, 21.81 μmol) and TEA (66.20 mg, 654.23 μmol). The mixture was stirred at rt for 16 h before it was concentrated. The resulting residue was purified by silica gel chromatography (petroleum ether:EtOAc=5:1) to provide the title compound (70 mg, yield: 84%) as colorless oil. MS (ESI) m/z=384.4 [M+H]⁺.

Step 6. Synthesis of tert-butyl trans-N-tert-butoxycarbonyl-N-[[3-[4-(2-pyridyl)pyrazol-1-yl]cyclobutyl]methyl]carbamate

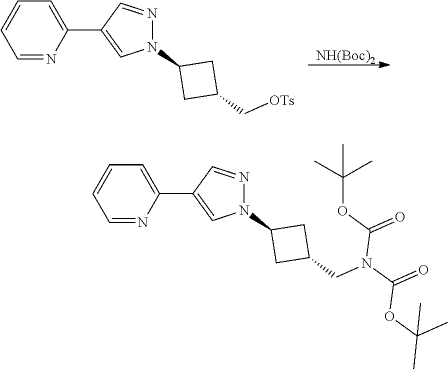 [SMILES: Cc1ccc(S(=O)(=O)OC[C@H]2C[C@H](n3cc(-c4ccccn4)cn3)C2)cc1 ; CC(C)(C)OC(=O)N(C[C@H]1C[C@H](n2cc(-c3ccccn3)cn2)C1)C(=O)OC(C)(C)C ; "NH(Boc)2"]

To a solution of (trans-3-(4-(pyridin-2-yl)-1H-pyrazol-1-yl)cyclobutyl)methyl 4-methylbenzenesulfonate (70 mg, 182.55 μmol) in DCM (10 mL) were added TsC₁ (52.03 mg, 273.82 μmol), DMAP (2.23 mg, 18.25 μmol) and TEA (55.42 mg, 547.64 μmol) at 0° C. The mixture was stirred at rt for 16 h before it was concentrated. The resulting residue was purified by silica gel chromatography (petroleum ether:EtOAc=5:1) to provide the title compound (70 mg, yield: 89%) as colorless oil. MS (ESI) m/z=429.4 [M+H]⁺.

Step 7. Synthesis of (trans-3-(4-(pyridin-2-yl)-1H-pyrazol-1-yl)cyclobutyl)methanamine

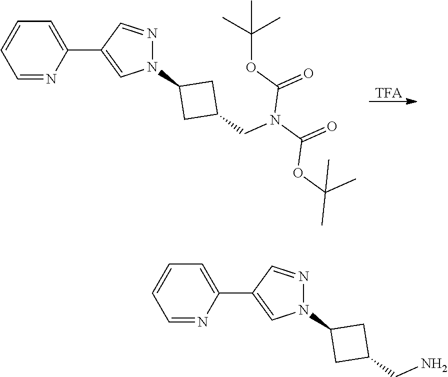 [SMILES: CC(C)(C)OC(=O)N(C[C@H]1C[C@H](n2cc(-c3ccccn3)cn2)C1)C(=O)OC(C)(C)C ; NC[C@H]1C[C@H](n2cc(-c3ccccn3)cn2)C1]

To a solution of tert-butyl trans-N-tert-butoxycarbonyl-N-[[3-[4-(2-pyridyl)pyrazol-1-yl]cyclobutyl]methyl]carbamate (70 mg, 163.35 μmol) in DCM (1 mL) was added TFA (1 mL). The mixture was stirred at rt for 30 min, before it was concentrated to provide the title compound (36 mg, yield: 97%) as colorless oil. MS (ESI) m/z=229.4 [M+H]⁺.

Step 8. Synthesis of 2-(2,6-dioxopiperidin-3-yl)-5-(((trans-3-(4-(pyridin-2-yl)-1H-pyrazol-1-yl)cyclobutyl)methyl)amino)isoindoline-1,3-dione

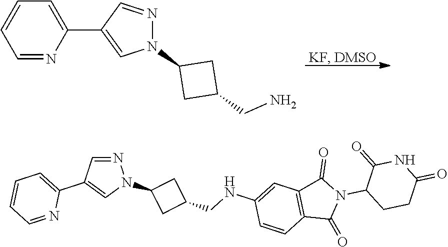 [SMILES: NC[C@H]1C[C@H](n2cc(-c3ccccn3)cn2)C1 ; O=C1CCC(N2C(=O)c3ccc(NC[C@H]4C[C@H](n5cc(-c6ccccn6)cn5)C4)cc3C2=O)C(=O)N1]

To a solution of (trans-3-(4-(pyridin-2-yl)-1H-pyrazol-1-yl)cyclobutyl)methanamine (50 mg, 219.02 μmol) in DMSO (1 mL) were added 2-(2,6-dioxo-3-piperidyl)-5-fluoro-isoindoline-1,3-dione (120.99 mg, 438.04 μmol) and KF (25.45 mg, 438.04 μmol). The mixture was irradiated at 130° C. in the microwave reactor for 20 min. The resulting residue was purified by reverse phase chromatography (0.1% TFA in water:MeOH=1:1) to provide the title compound (17 mg, yield: 16%) as yellow solid. MS (ESI) m/z=485.4 [M+H]⁺.

### Example 536. 2-(2,6-Dioxopiperidin-3-yl)-5-(((trans-3-(4-(6-methylpyridin-2-yl)-1H-pyrazol-1-yl)cyclobutyl)methyl)amino)isoindoline-1,3-dione (GS-578)

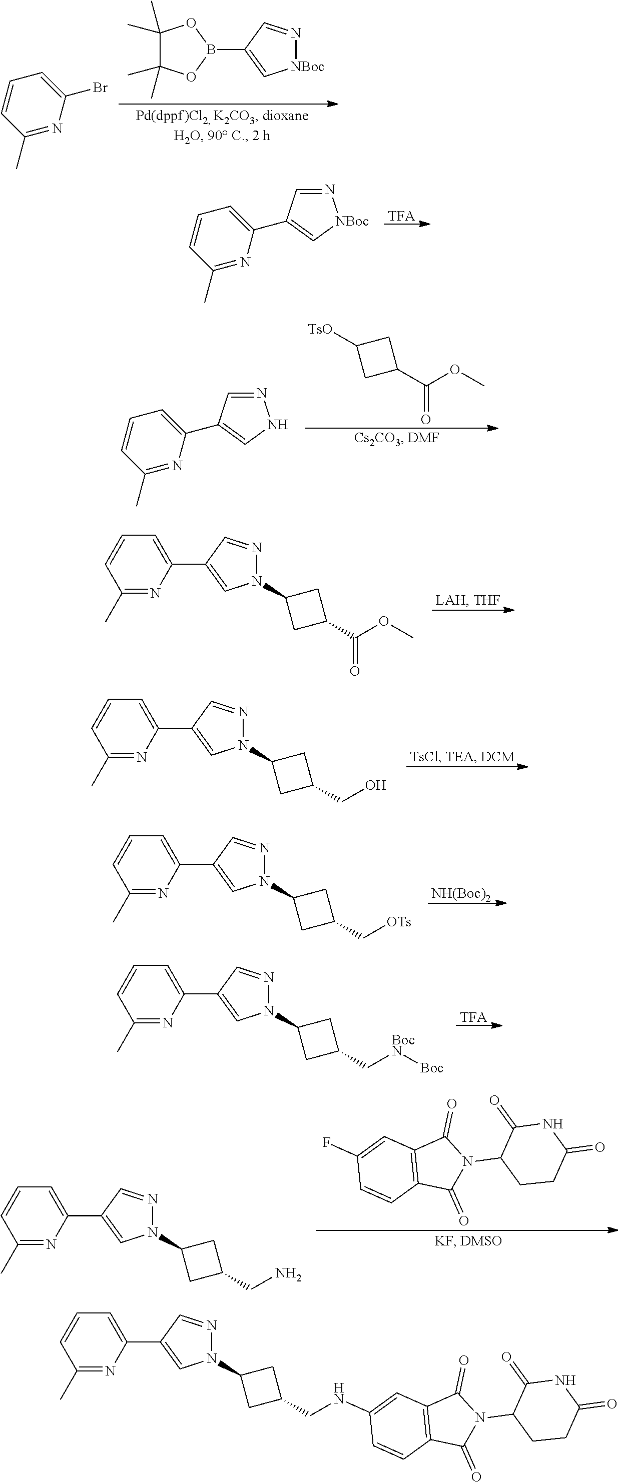

GS-578 was synthesized following the similar procedures as described for GS-577 (17 mg, yield over 8 steps: 2%) as yellow solid. MS (ESI) m/z=499.4 [M+H]⁺.

### Example 537. 5-(((trans-3-(3-Cyclopropyl-4-(quinoxalin-2-yl)-1H-pyrazol-1-yl)cyclobutyl)methyl)amino)-2-(2,6-dioxopiperidin-3-yl)isoindoline-1,3-dione (GS-579)

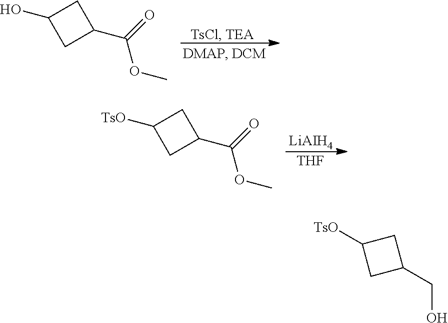

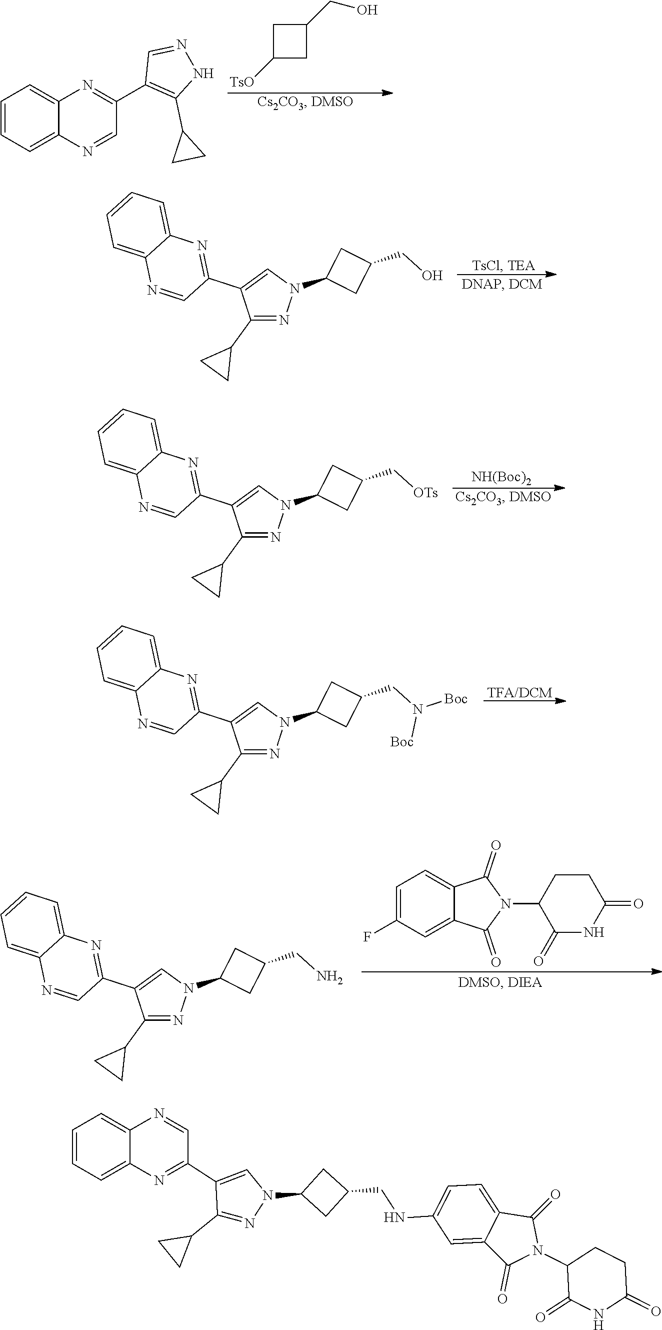

Step 1. Synthesis of methyl 3-(tosyloxy)cyclobutane-1-carboxylate

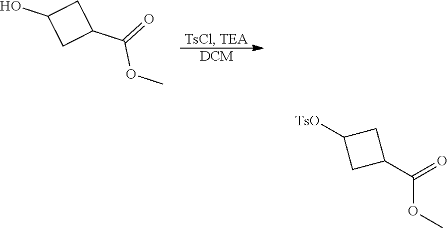

To a solution of methyl 3-hydroxycyclobutanecarboxylate (220 g, 1.69 mol) in DCM (1200 mL) were added DMAP (9 g, 73.67 mmol) and TEA (513.17 g, 5.07 mol). 4-Methylbenzenesulfonyl chloride (386.74 g, 2.03 mol) was added in portions at 25° C. The mixture was stirred for 18 h, before it was poured into ice/water (1000 mL). The organic layer was separated and washed with aq. Na₂CO₃ solution (10%, 1000 mL×2). The organic phase was concentrated in vacuo to provide the title compound (480 g, yield: 99%) as brown oil, which was used directly in the next step.

Step 2. Synthesis of 3-(hydroxymethyl)cyclobutyl 4-methylbenzenesulfonate

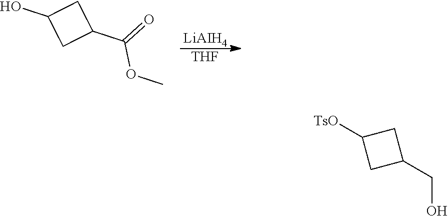

To a suspension of LiAlH₄ (12.03 g, 316.54 mmol) in THF (500 mL) was added methyl 3-(tosyloxy)cyclobutane-1-carboxylate (75 g, 263.78 mmol) dropwise at 25° C. The mixture was stirred at 25° C. for 10 min, before it was quenched with water (50 mL). The mixture was filtered. The filter cake was washed with THF (500 mL). The combined filtrate was concentrated in vacuo to provide the title compound (65 g, yield: 96%) as yellow oil.

Step 3. Synthesis of (trans-3-(3-cyclopropyl-4-(quinoxalin-2-yl)-1H-pyrazol-1-yl)cyclobutyl)methanol

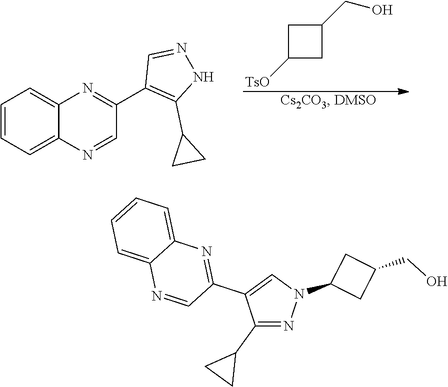

To a mixture of 2-(5-cyclopropyl-1H-pyrazol-4-yl)quinoxaline (see step 5 of GS-568 for synthesis, 10 g, 38.09 mmol), Cs₂CO₃ (18.24 g, 56 mmol) and 3-(hydroxymethyl)cyclobutyl 4-methylbenzenesulfonate (12.20 g, 47.61 mmol) in DMSO (60 mL) was heated at 90° C. for 3 h. The mixture was cooled to rt, and filtered. The organic phase was purified by reverse phase chromatography to afford the crude product, which was slurried in MTBE, and filtered to provide the title compound (4.3 g, yield: 35%) as grey solid. MS (ESI) m/z=321.3 [M+H]⁺.

Step 4. Synthesis of (trans-3-(3-cyclopropyl-4-(quinoxalin-2-yl)-1H-pyrazol-1-yl)cyclobutyl)methyl 4-methylbenzenesulfonate

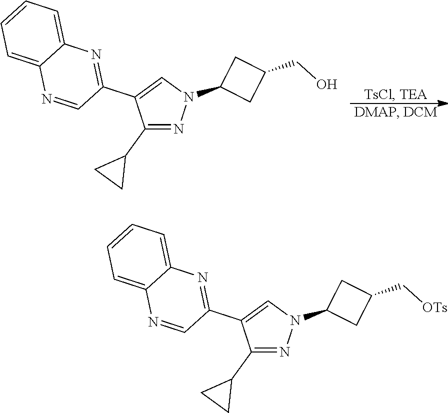

To a solution of (trans-3-(3-cyclopropyl-4-(quinoxalin-2-yl)-1H-pyrazol-1-yl)cyclobutyl)methanol (4.3 g, 13.42 mmol) in DCM (60 mL) were added TEA (4.07 g, 40.26 mmol) and DMAP (0.5 g, 4.09 mmol). The mixture was stirred at rt for 10 min, before 4-methylbenzenesulfonyl chloride (3.33 g, 17.45 mmol) was added slowly. The mixture was stirred overnight, before it was concentrated. The resulting residue was purified by silica gel chromatography to provide the title compound (6.0 g, yield: 89%) as light-yellow solid. MS (ESI) m/z=475.3 [M+H]⁺.

Step 5. Synthesis of tert-butyl trans-N-tert-butoxycarbonyl-N-[[trans-3-(3-cyclopropyl-4-quinoxalin-2-yl-pyrazol-1-yl)cyclobutyl]methyl]carbamate

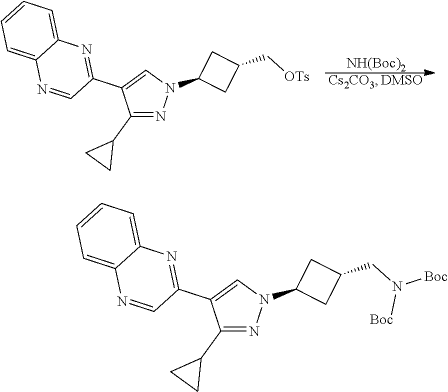

To a solution of (trans-3-(3-cyclopropyl-4-(quinoxalin-2-yl)-1H-pyrazol-1-yl)cyclobutyl)methyl 4-methylbenzenesulfonate (6.0 g, 12.01 mmol) in DMF (25 mL) were added tert-butyl N-tert-butoxycarbonylcarbamate (3.13 g, 14.41 mmol) and Cs₂CO₃ (7.66 g, 23.52 mmol) at rt. The mixture was heated at 90° C. for 1 h. After the reaction was cooled to rt, it was poured into water (30 mL), and extracted with EtOAc (30 mL×2). The combined organic layers were washed with water (30 mL), dried and concentrated to provide the title compound (6.1 g, yield: 93%) as light-yellow oil. MS (ESI) m/z=520.4 [M+H]⁺.

Step 6. Synthesis of (trans-3-(3-cyclopropyl-4-(quinoxalin-2-yl)-1H-pyrazol-1-yl)cyclobutyl)methanamine

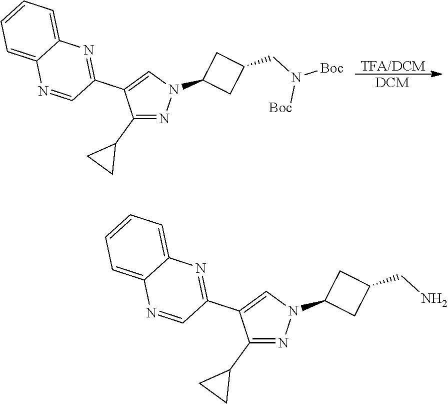 [SMILES: CC(C)(C)OC(=O)N(C[C@H]1C[C@H](n2cc(-c3cnc4ccccc4n3)c(C3CC3)n2)C1)C(=O)OC(C)(C)C ; NC[C@H]1C[C@H](n2cc(-c3cnc4ccccc4n3)c(C3CC3)n2)C1]

To a solution of tert-butyl trans-N-tert-butoxycarbonyl-N-[[trans-3-(3-cyclopropyl-4-quinoxalin-2-yl-pyrazol-1-yl)cyclobutyl]methyl]carbamate (6.1 g, 11.15 mmol) in DCM (60 mL) was added TFA (20 mL) at rt. The mixture was stirred at rt for 2 h. The mixture was concentrated, and the resulting residue was added to water (60 mL). The pH was adjusted to 10-11 by addition of sat. Na₂CO₃ solution. The obtained mixture was extracted with EtOAc (60 mL×2). The combined organic layers were dried and concentrated to provide the title compound (3.3 g, yield: 88%) as brown oil. MS (ESI) m/z=320.4 [M+H]⁺.

Step 7. Synthesis of 5-(((trans-3-(3-cyclopropyl-4-(quinoxalin-2-yl)-1H-pyrazol-1-yl)cyclobutyl)methyl)amino)-2-(2,6-dioxopiperidin-3-yl)isoindoline-1,3-dione

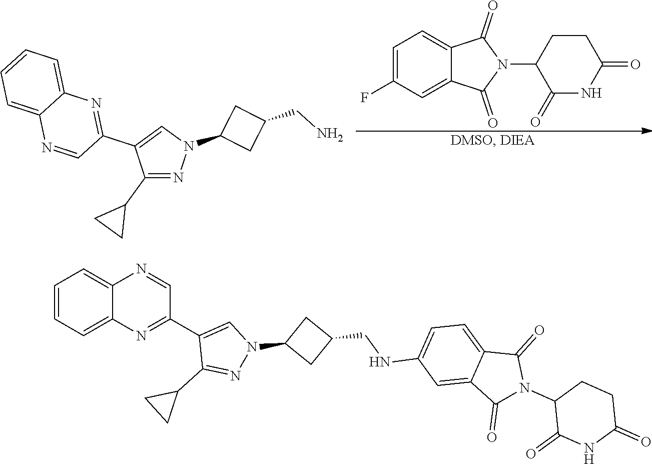

A mixture of (trans-3-(3-cyclopropyl-4-(quinoxalin-2-yl)-1H-pyrazol-1-yl)cyclobutyl)methanamine (3.2 g, 9.52 mmol), 2-(2,6-dioxo-3-piperidyl)-5-fluoro-isoindoline-1,3-dione (3.65 g, 13.21 mmol) and N-ethyl-N-isopropyl-propan-2-amine (3.08 g, 23.79 mmol, 3.93 mL) in DMSO (35 mL) was heated at 150° C. under N₂ for 1.5 h. The mixture was purified by reverse phase chromatography to provide the title compound (1.45 g, yield: 26%) as yellow solid. MS (ESI) m/z=576.7 [M+H]⁺.

### Example 538. 5-((3-(trans-3-(4-(7-(4-Cyclobutylpiperazin-1-yl)quinoxalin-2-yl)-1H-pyrazol-1-yl)cyclobutyl)propyl)amino)-2-(2,6-dioxopiperidin-3-yl)isoindoline-1,3-dione (GS-580)

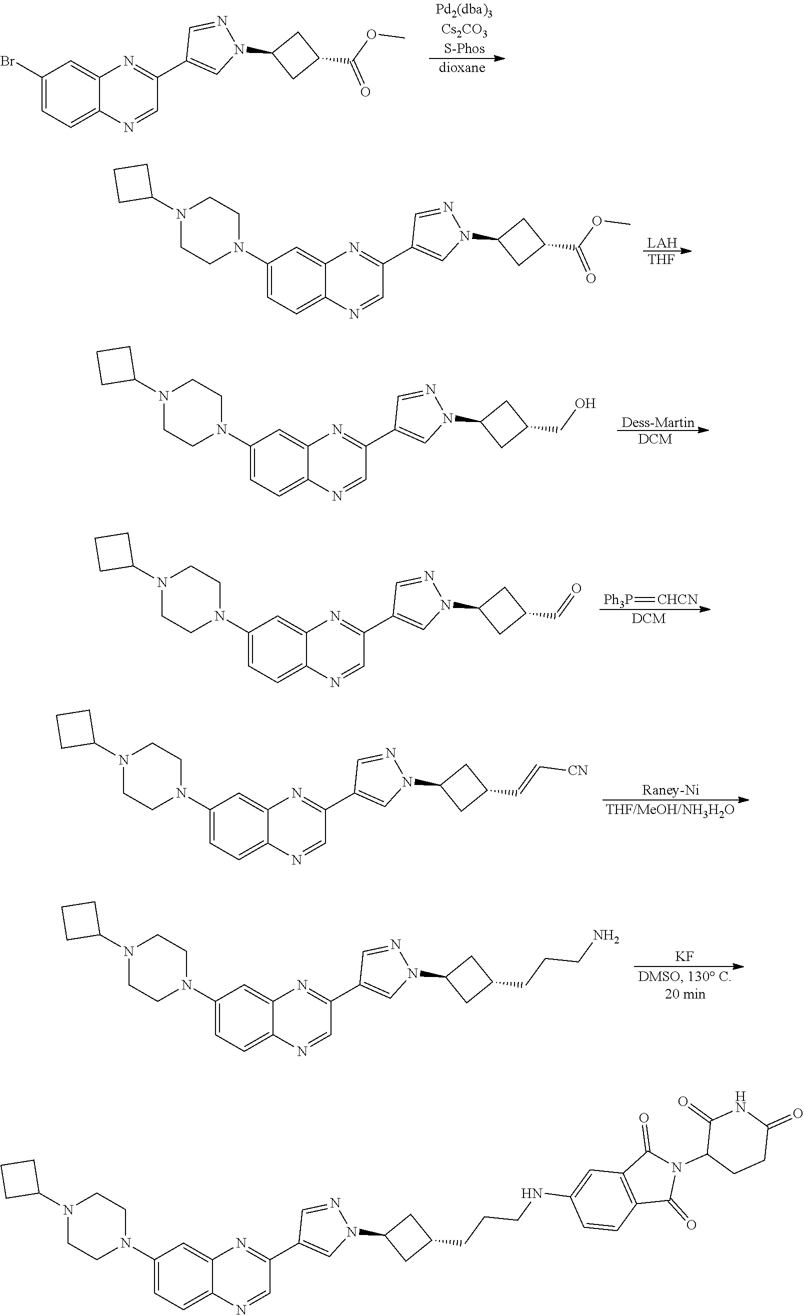

GS-580 was synthesized following the similar procedures as described for GS-399 (18 mg, yield over 6 steps: 6%) as yellow solid. MS (ESI) m/z=702.7 [M+H]⁺.

### Example 539. 5-((3-(trans-3-(4-(7-((R)-3,4-Dimethylpiperazin-1-yl)quinoxalin-2-yl)-1H-pyrazol-1-yl)cyclobutyl)propyl)amino)-2-(2,6-dioxopiperidin-3-yl)isoindoline-1,3-dione (GS-581)

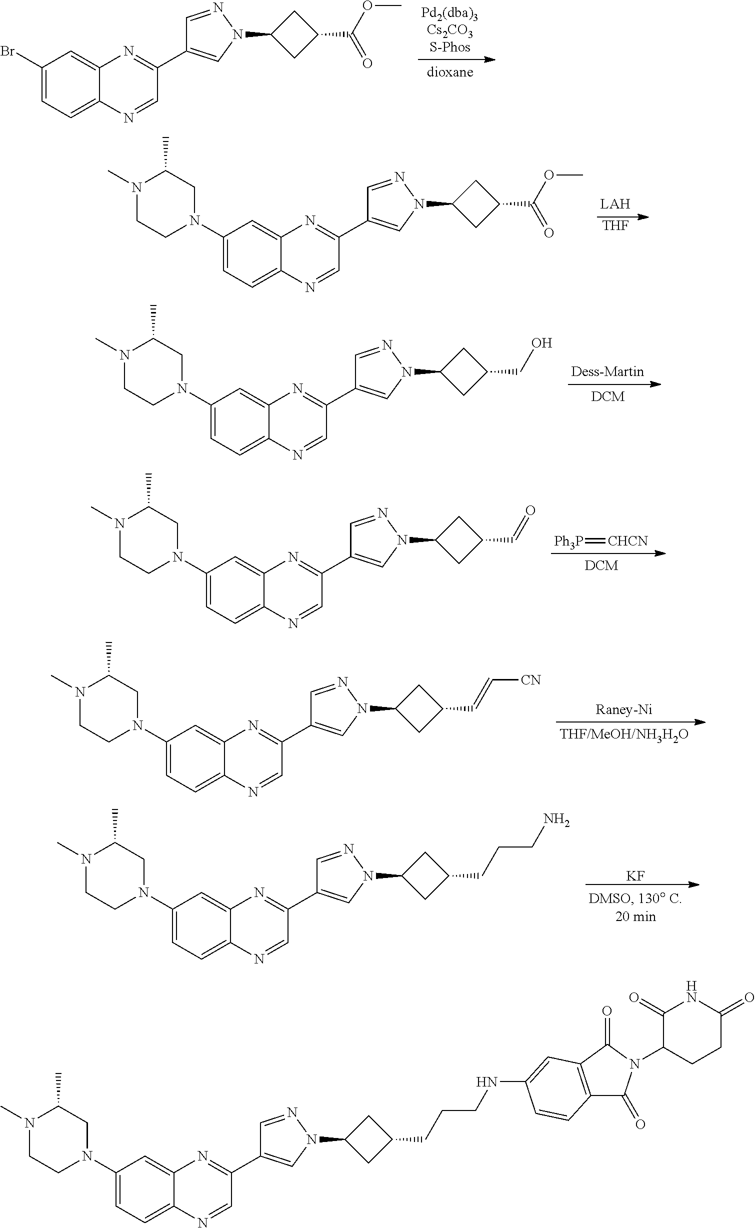

GS-581 was synthesized following the similar procedures as described for GS-399 (8.1 mg, yield over 6 steps: 3%) as yellow solid. MS (ESI) m/z=676.6 [M+H]⁺.

### Example 540. 2-(2,6-Dioxopiperidin-3-yl)-5-((3-(trans-3-(4-(pyridin-2-yl)-1H-pyrazol-1-yl)cyclobutyl)propyl)amino)isoindoline-1,3-dione (GS-582)

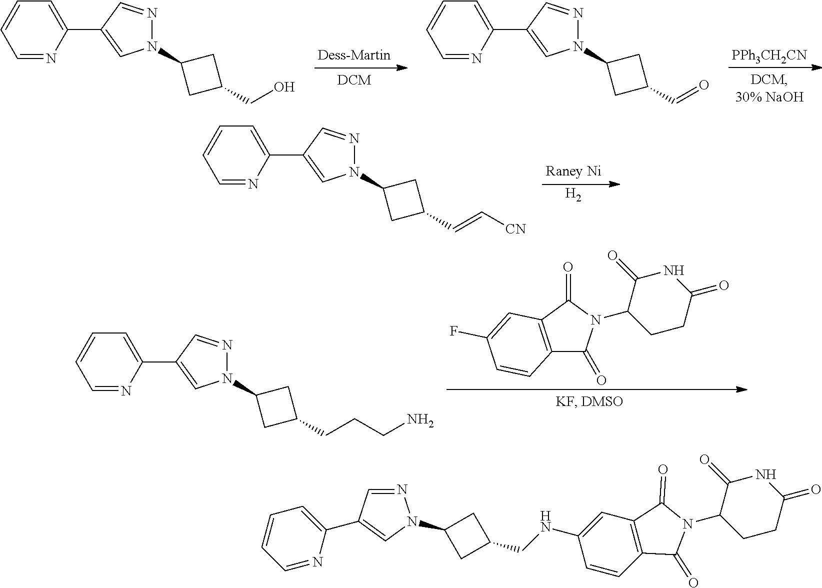

GS-582 was synthesized following the similar procedures as described for steps 8 to 11 of GS-568 (15 mg, yield over 4 steps: 7%) as yellowish solid. MS (ESI) m/z=513.5 [M+H]⁺.

### Example 541. 2-(2,6-Dioxopiperidin-3-yl)-5-((3-(trans-3-(4-(7-(hexahydropyrrolo[1,2-a]pyrazin-2(1H)-yl)quinoxalin-2-yl)-1H-pyrazol-1-yl)cyclobutyl)propyl)amino)isoindoline-1,3-dione (GS-583)

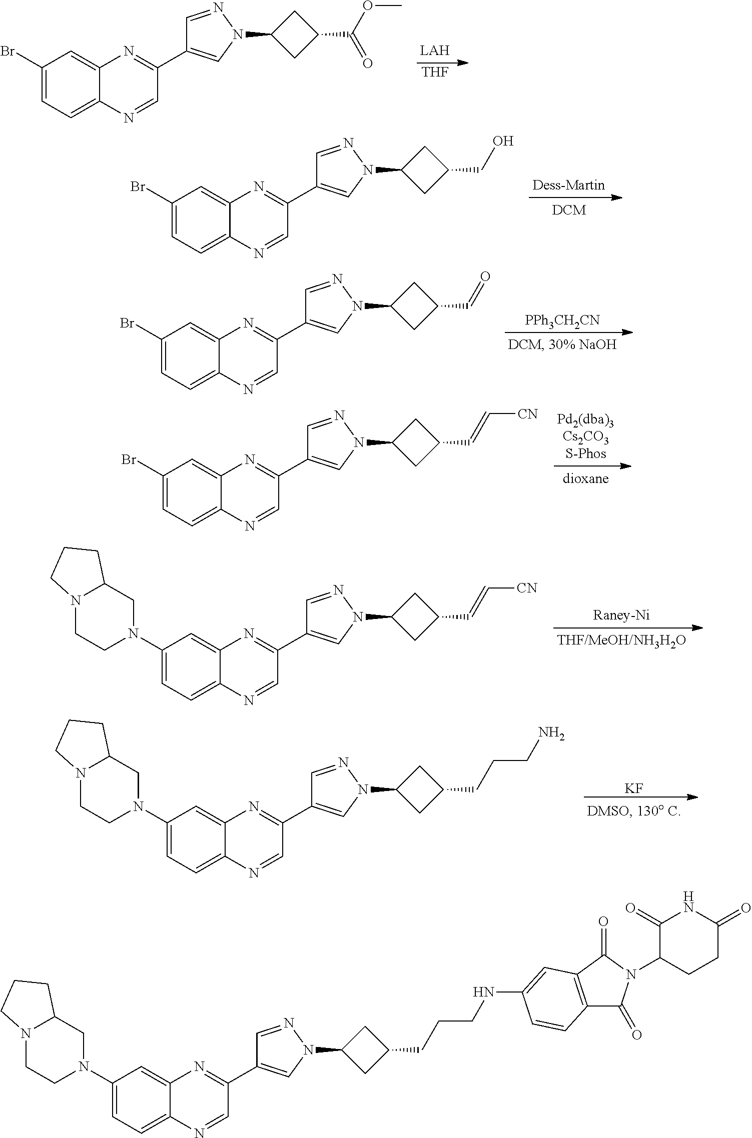

GS-583 was synthesized following the similar procedures as described for GS-567 (8.8 mg, yield over 6 steps: 410) as yellow solid. MS (ESI) m/z=688.6 [M+H]⁺.

### Example 542. 2-(2,6-Dioxopiperidin-3-yl)-5-((3-(trans-3-(4-(6-methylpyridin-2-yl)-1H-pyrazol-1-yl)cyclobutyl)propyl)amino)isoindoline-1,3-dione (GS-584)

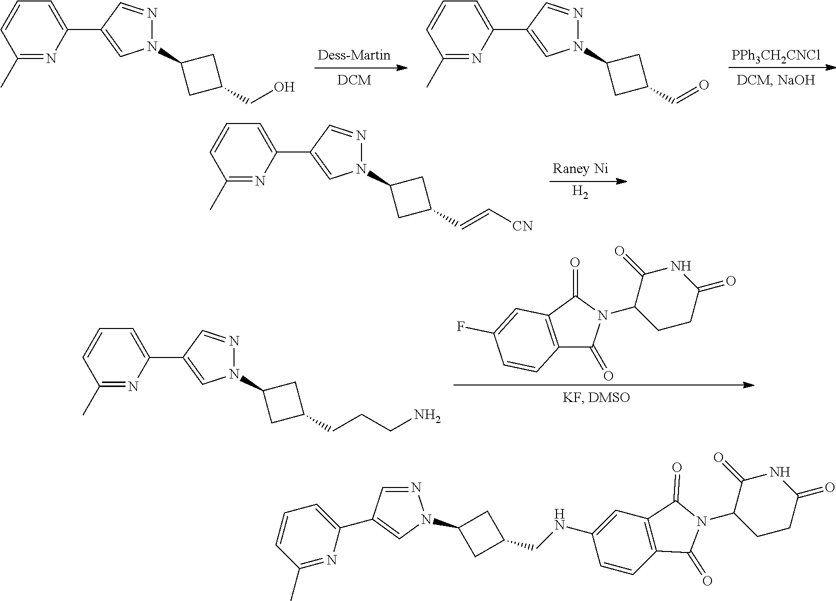

GS-584 was synthesized following the similar procedures as described for steps 8 to 11 of GS-568 (26 mg, yield over 4 steps: 10%) as yellowish solid. MS (ESI) m/z=527.5 [M+H]⁺.

### Example 543. 2-(2,6-Dioxopiperidin-3-yl)-5-((3-(trans-3-(4-(7-(oxetan-3-ylamino)quinoxalin-2-yl)-1H-pyrazol-1-yl)cyclobutyl)propyl)amino)isoindoline-1,3-dione (GS-585)

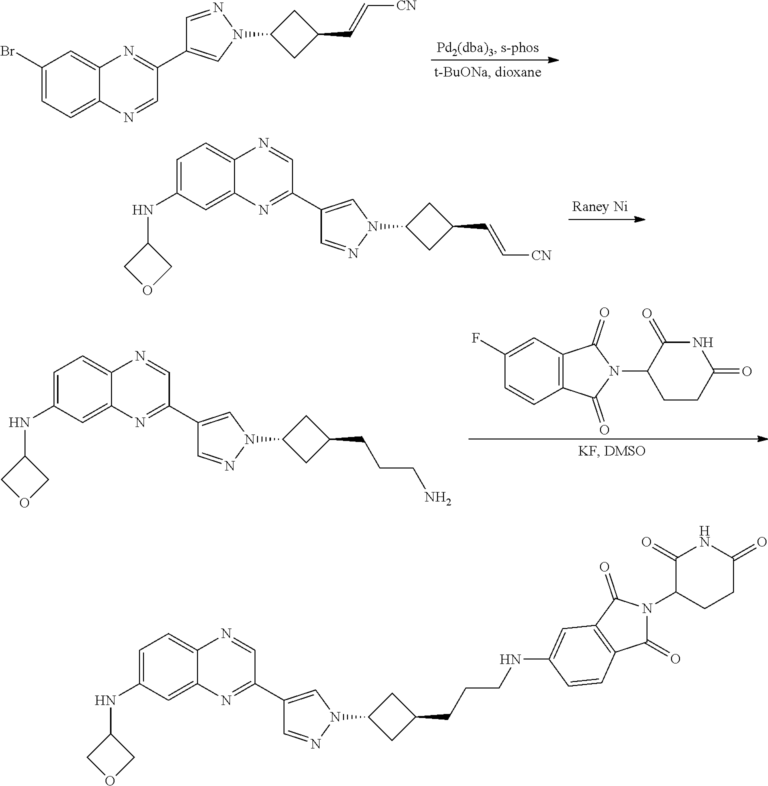 [SMILES: N#C/C=C/[C@H]1C[C@H](n2cc(-c3cnc4ccc(Br)cc4n3)cn2)C1 ; N#C/C=C/[C@H]1C[C@H](n2cc(-c3cnc4ccc(NC5COC5)cc4n3)cn2)C1 ; NCCC[C@H]1C[C@H](n2cc(-c3cnc4ccc(NC5COC5)cc4n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccc(NC8COC8)cc7n6)cn5)C4)cc3C2=O)C(=O)N1]

Step 1. Synthesis of 3-(trans-3-(4-(7-(oxetan-3-ylamino)quinoxalin-2-yl)-H-pyrazol-1-yl)cyclobutyl)acrylonitrile

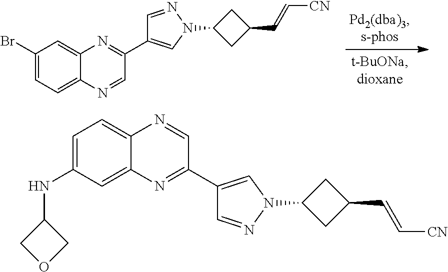

To a solution of 3-(trans-3-(4-(7-bromoquinoxalin-2-yl)-1H-pyrazol-1-yl)cyclobutyl)acrylonitrile (synthesized by a similar procedure for step 9 of GS-568, 80 mg, 210.39 μmol) in dioxane (2 mL) were added oxetan-3-amine (30.76 mg, 420.79 μmol), Pd₂(dba)₃ (19.25 mg, 21.04 μmol), s-phos (17.25 mg, 42.08 μmol) and Cs₂CO₃ (136.76 mg, 420.79 μmol). After the mixture was stirred at 90° C. for 1 h, it was purified by reverse phase chromatography (water:MeOH=1:1) to provide the title compound (75 mg, yield: 96%) as yellowish oil. MS (ESI) m/z=373.4 [M+H]⁺.

Step 2. Synthesis of 3-(1-(trans-3-(3-aminopropyl)cyclobutyl)-1H-pyrazol-4-yl)-N-(oxetan-3-yl)quinoxalin-6-amine

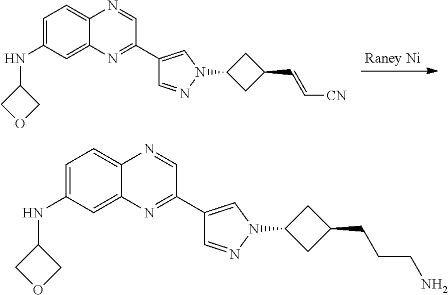 [SMILES: N#C/C=C/[C@H]1C[C@H](n2cc(-c3cnc4ccc(NC5COC5)cc4n3)cn2)C1 ; NCCC[C@H]1C[C@H](n2cc(-c3cnc4ccc(NC5COC5)cc4n3)cn2)C1]

To a solution of 3-(trans-3-(4-(7-(oxetan-3-ylamino)quinoxalin-2-yl)-1H-pyrazol-1-yl)cyclobutyl)acrylonitrile (100 mg, 268.51 μmol) in NH₃/MeOH (2 mL) and THF (5 mL) was added Raney Ni (100 mg). The mixture was stirred at rt for 1 h under H₂ atmosphere. Then, the mixture was filtered, and the filtrate was concentrated to provide the title compound, which was used directly in the next step. MS (ESI) m/z=379.4 [M+H]⁺.

Step 3. Synthesis of 2-(2,6-dioxopiperidin-3-yl)-5-((3-(trans-3-(4-(7-(oxetan-3-ylamino)quinoxalin-2-yl)-1H-pyrazol-1-yl)cyclobutyl)propyl)amino)isoindoline-1,3-dione

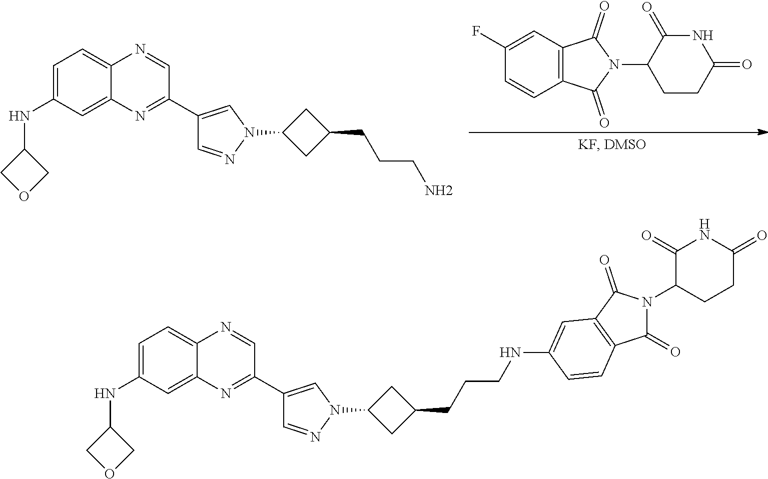 [SMILES: NCCC[C@H]1C[C@H](n2cc(-c3cnc4ccc(NC5COC5)cc4n3)cn2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6cnc7ccc(NC8COC8)cc7n6)cn5)C4)cc3C2=O)C(=O)N1]

To a solution of 3-(1-(trans-3-(3-aminopropyl)cyclobutyl)-1H-pyrazol-4-yl)-N-(oxetan-3-yl)quinoxalin-6-amine (50 mg, 132.11 μmol) in DMSO (1 mL) were added 2-(2,6-dioxo-3-piperidyl)-5-fluoro-isoindoline-1,3-dione (72.98 mg, 264.22 μmol) and KF (15.35 mg, 264.22 μmol). The mixture was irradiated at 130° C. in the microwave reactor for 20 min. The mixture was purified by reverse phase chromatography (water:MeOH=1:1) to provide the title compound (21 mg, yield: 25%) as yellow solid. MS (ESI) m/z=635.5 [M+H]⁺.

### Example 544. 5-((3-(Cis-3-(3-cyclopropyl-4-(6-(oxetan-3-ylamino)quinoxalin-2-yl)-1H-pyrazol-1-yl)cyclobutyl)propyl)amino)-2-(2,6-dioxopiperidin-3-yl)isoindoline-1,3-dione (GS-586)

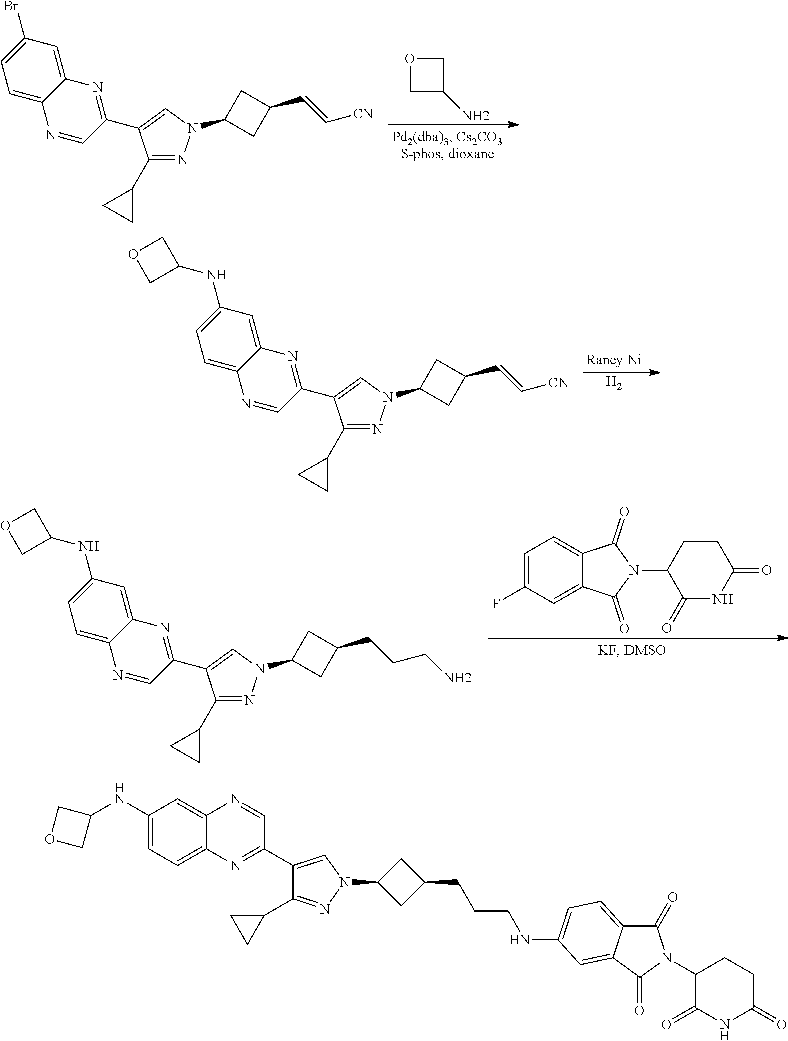

GS-586 was synthesized following the similar procedures as described for GS-585 (4 mg) as yellow solid. MS (ESI) m/z=675.6 [M+H]⁺.

### Example 545. 2-(2,6-Dioxopiperidin-3-yl)-5-((3-(trans-3-(4-(7-(4-(2,2,2-trifluoroethyl)piperazin-1-yl)quinoxalin-2-yl)-1H-pyrazol-1-yl)cyclobutyl)propyl)amino)isoindoline-1,3-dione (GS-587)

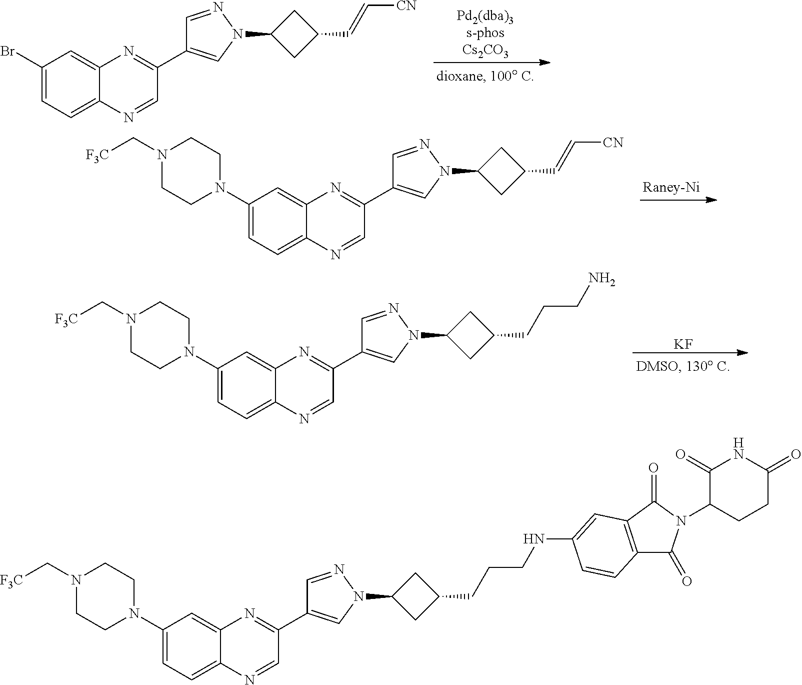

GS-587 was synthesized following the similar procedures as described for GS-585 (1.3 mg, yield over 3 steps: 1%) as yellow solid. MS (ESI) m/z=730.7 [M+H]⁺.

### Example 546 and 547. 5-(((trans-3-(3-Cyclopropyl-1H-pyrazol-1-yl)cyclobutyl)methyl)amino)-2-(2,6-dioxopiperidin-3-yl)isoindoline-1,3-dione (GS-588) and 5-(((trans-3-(5-cyclopropyl-1H-pyrazol-1-yl)cyclobutyl)methyl)amino)-2-(2,6-dioxopiperidin-3-yl)isoindoline-1,3-dione (GS-590)

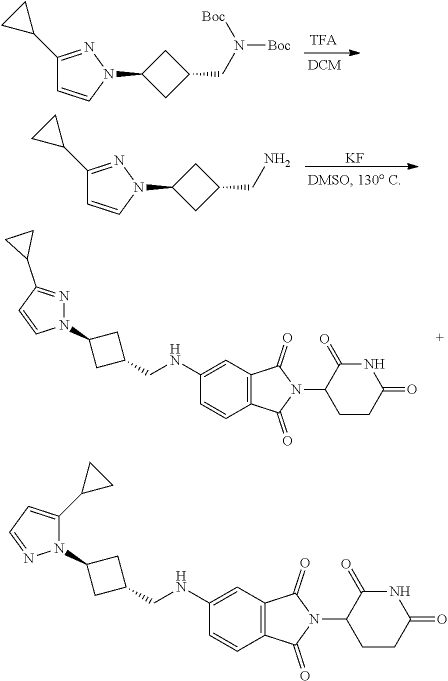

GS-588 and GS-590 were synthesized following the similar procedures as described for GS-579 (GS-588, 13.1 mg, yellow solid, MS (ESI) m/z=448.6 [M+H]⁺; GS-590, 2.6 mg, yellow solid, MS (ESI) m/z=448.6 [M+H]⁺).

### Example 548. 5-(((trans-3-(3-Cyclopropyl-4-(6-methylpyridin-2-yl)-1H-pyrazol-1-yl)cyclobutyl)methyl)amino)-2-(2,6-dioxopiperidin-3-yl)isoindoline-1,3-dione ((GS-589)

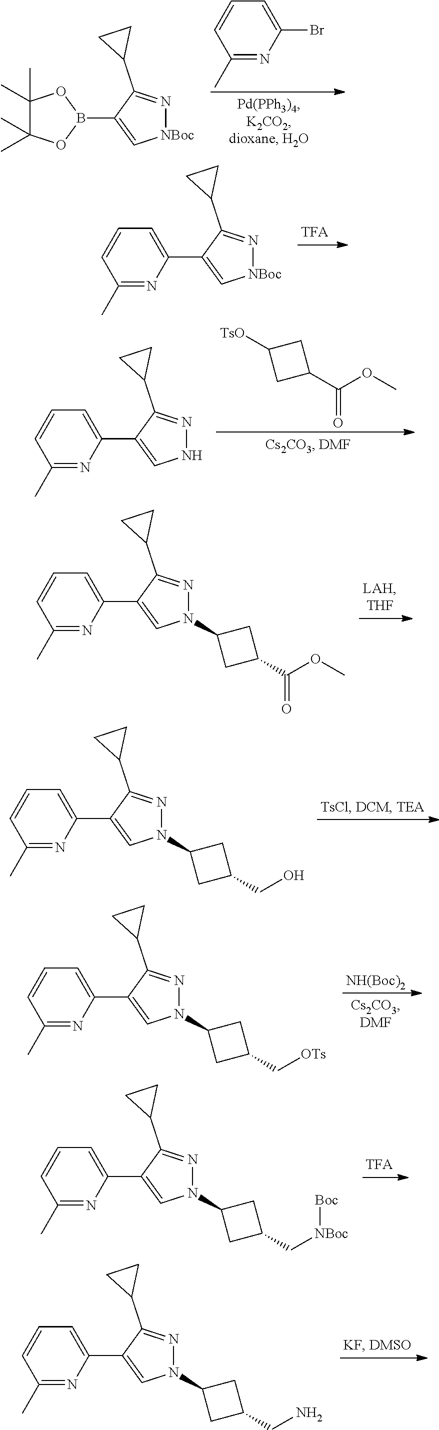

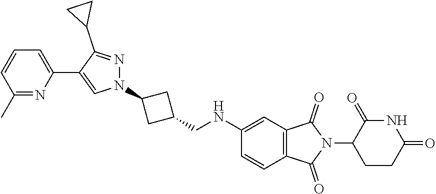 [SMILES: Cc1cccc(-c2cn([C@H]3C[C@H](CNc4ccc5c(c4)C(=O)N(C4CCC(=O)NC4=O)C5=O)C3)nc2C2CC2)n1]

GS-589 was synthesized following the similar procedures as described for GS-577 (12 mg, yield over 8 steps: 1%) as yellowish solid. MS (ESI) m/z=539.5 [M+H]⁺.

### Example 549. 5-((3-(trans-3-(3-Cyclopropyl-1H-pyrazol-1-yl)cyclobutyl)propyl)amino)-2-(2,6-dioxopiperidin-3-yl)isoindoline-1,3-dione ((GS-591)

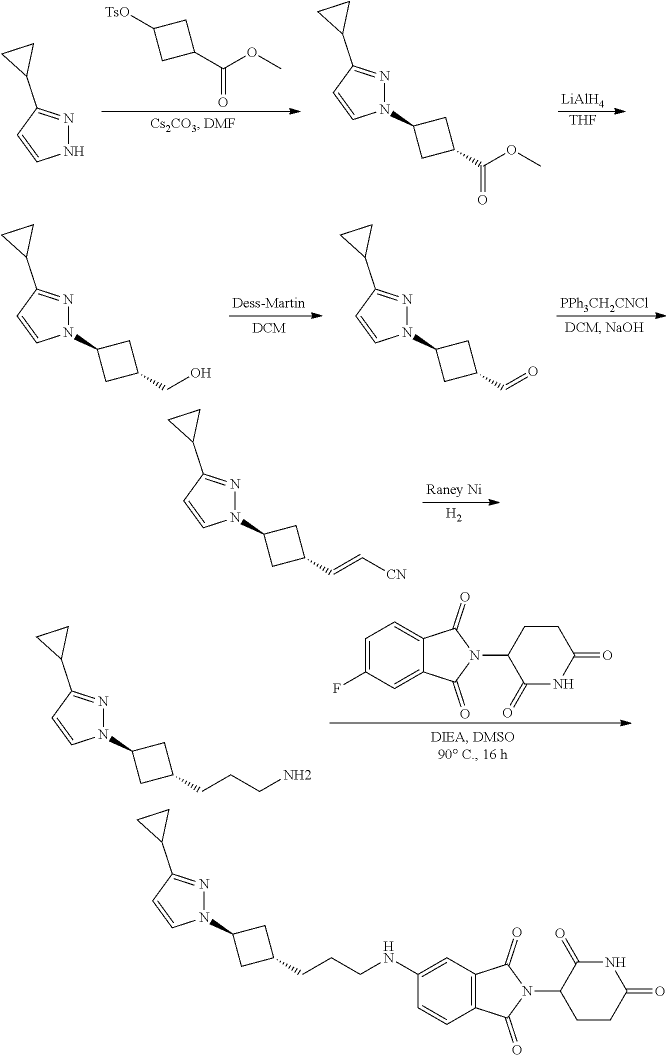

Step 1. Synthesis of methyl trans-3-(3-cyclopropyl-TH-pyrazol-1-yl)cyclobutane-1-carboxylate

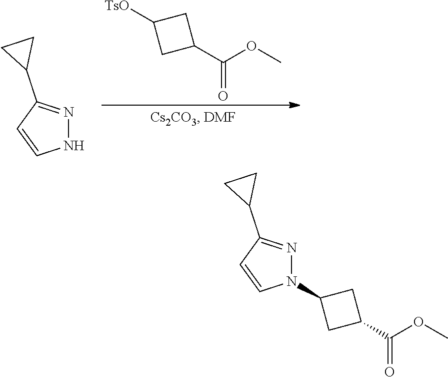 [SMILES: c1cc(C2CC2)n[nH]1 ; COC(=O)C1CC(OS(=O)(=O)c2ccc(C)cc2)C1 ; COC(=O)[C@H]1C[C@H](n2ccc(C3CC3)n2)C1]

To a solution of 3-cyclopropyl-1H-pyrazole (1 g, 9.25 mmol) in DMF (10 mL) were added methyl 3-(p-tolylsulfonyloxy)cyclobutanecarboxylate (3.16 g, 11.10 mmol) and Cs₂CO₃ (6.01 g, 18.49 mmol). After the mixture was stirred at 100° C. for 16 h, it was quenched with water (20 mL), and extracted with EtOAc (20 mL). The organic phase was concentrated. The resulting residue was purified by silica gel chromatography (petroleum ether:EtOAc=3:1) to provide the title compound (300 mg, yield: 15%) as colorless oil. MS (ESI) m/z=221.2 [M+H]⁺.

Step 2. Synthesis of (trans-3-(3-cyclopropyl-1H-pyrazol-1-yl)cyclobutyl)methanol

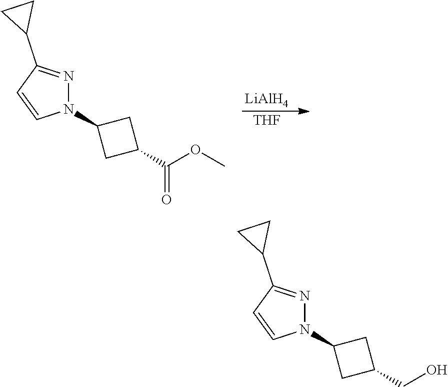 [SMILES: COC(=O)[C@H]1C[C@H](n2ccc(C3CC3)n2)C1 ; "LiAlH4" ; OC[C@H]1C[C@H](n2ccc(C3CC3)n2)C1 ; "THF"]

To a solution of methyl trans-3-(3-cyclopropyl-1H-pyrazol-1-yl)cyclobutane-1-carboxylate (300 mg, 1.36 mmol) in THF (10 mL) was added LiAlH (46.20 mg, 1.36 mmol). After the mixture was stirred at rt for 1 h, it was quenched with water, and filtered. The filtrate was concentrated to provide the title compound (253 mg, yield: 97%) as yellow oil. MS (ESI) m/z=193.3 [M+H]⁺.

Step 3. Synthesis of trans-3-(3-cyclopropyl-1H-pyrazol-1-yl)cyclobutane-1-carbaldehyde

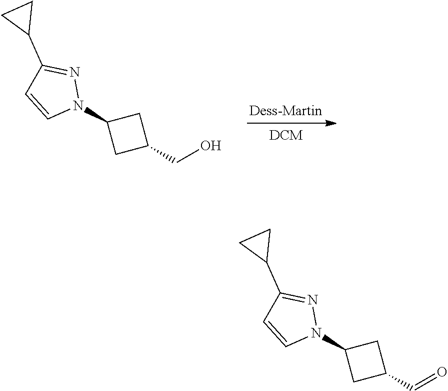 [SMILES: OC[C@H]1C[C@H](n2ccc(C3CC3)n2)C1 ; O=C[C@H]1C[C@H](n2ccc(C3CC3)n2)C1]

To a solution of (trans-3-(3-cyclopropyl-1H-pyrazol-1-yl)cyclobutyl)methanol (200 mg, 1.04 mmol) in DCM (10 mL) was added DMP (882.15 mg, 2.08 mmol). After the reaction was stirred at rt for 1 h, the mixture was filtered, and washed with water (20 mL). The filtrate was concentrated to provide the title compound (160 mg, yield: 81%) as yellow oil. MS (ESI) m/z=191.3 [M+H]⁺.

Step 4. Synthesis of 3-(trans-3-(3-cyclopropyl-1H-pyrazol-1-yl)cyclobutyl)acrylonitrile

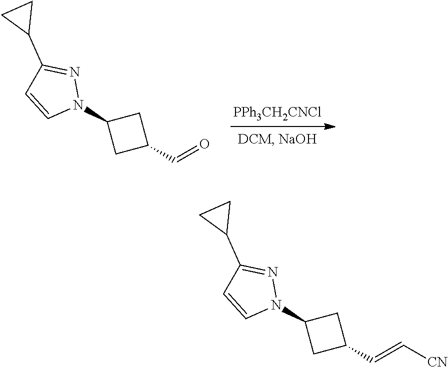 [SMILES: O=C[C@H]1C[C@H](n2ccc(C3CC3)n2)C1 ; N#C/C=C/[C@H]1C[C@H](n2ccc(C3CC3)n2)C1]

To a solution of trans-3-(3-cyclopropyl-1H-pyrazol-1-yl)cyclobutane-1-carbaldehyde (140 mg, 735.91 μmol) in DCM (10 mL) was added (cyanomethyl)triphenylphosphonium chloride (298.29 mg, 883.09 μmol) and aq. NaOH solution (30%, 1 mL). After the mixture was stirred at rt for 10 min, it was washed with water (10 mL). The organic phase was concentrated. The resulting residue was purified by reverse phase chromatography (water:MeOH=1:1) to provide the title compound (87 mg, yield: 55%) as yellow oil. MS (ESI) m/z=214.2 [M+H]⁺.

Step 5. Synthesis of 3-(trans-3-(3-cyclopropyl-1H-pyrazol-1-yl)cyclobutyl)propan-1-amine

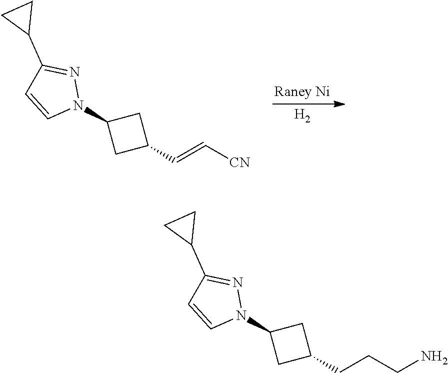 [SMILES: N#C/C=C/[C@H]1C[C@H](n2ccc(C3CC3)n2)C1 ; "H2" ; NCCC[C@H]1C[C@H](n2ccc(C3CC3)n2)C1]

To a solution of 3-(trans-3-(3-cyclopropyl-1H-pyrazol-1-yl)cyclobutyl)acrylonitrile (87 mg, 407.92 μmol) in methanol (10 mL) were added THF (1 mL) and Raney Ni (8 mg). After the mixture was stirred at rt for 1 h under H₂, it was filtered. The filtrate was concentrated. The resulting residue was purified by reverse phase chromatography (water:MeOH=1:1) to provide the title compound (76 mg, yield: 85%) as colorless oil. MS (ESI) m/z=219.3 [M+H]⁺.

Step 6. Synthesis of 5-((3-(trans-3-(3-cyclopropyl-1H-pyrazol-1-yl)cyclobutyl)propyl)amino)-2-(2,6-dioxopiperidin-3-yl)isoindoline-1,3-dione

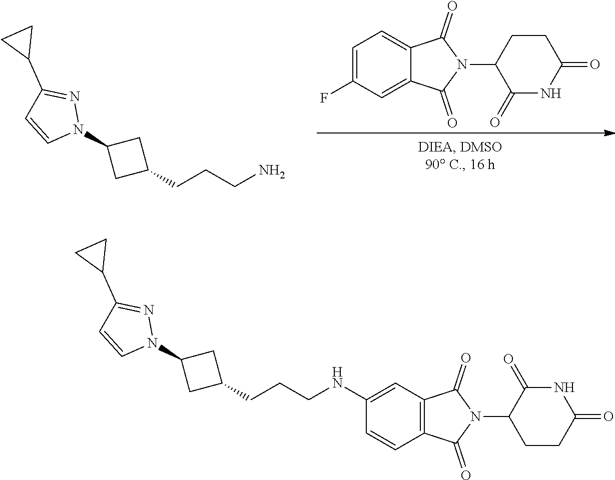 [SMILES: NCCC[C@H]1C[C@H](n2ccc(C3CC3)n2)C1 ; O=C1CCC(N2C(=O)c3ccc(F)cc3C2=O)C(=O)N1 ; O=C1CCC(N2C(=O)c3ccc(NCCC[C@H]4C[C@H](n5ccc(C6CC6)n5)C4)cc3C2=O)C(=O)N1]

To a solution of 3-(trans-3-(3-cyclopropyl-1H-pyrazol-1-yl)cyclobutyl)propan-1-amine (76 mg, 346.52 μmol) in DMSO (2 mL) were added 2-(2,6-dioxo-3-piperidyl)-5-fluoro-isoindoline-1,3-dione (114.86 mg, 415.82 μmol) and DIPEA (89.57 mg, 693.04 μmol, 114.54 μL). The mixture was stirred at 110° C. for 16 h. The mixture was purified by reverse phase chromatography (water:MeOH=1:1) to provide the title compound (20 mg) as yellowish solid. MS (ESI) m/z=476.3 [M+H]⁺.

### Example 550. 5-((3-(trans-3-(3-Cyclopropyl-4-(6-methylpyridin-2-yl)-1H-pyrazol-1-yl)cyclobutyl)propyl)amino)-2-(2,6-dioxopiperidin-3-yl)isoindoline-1,3-dione (GS-592)

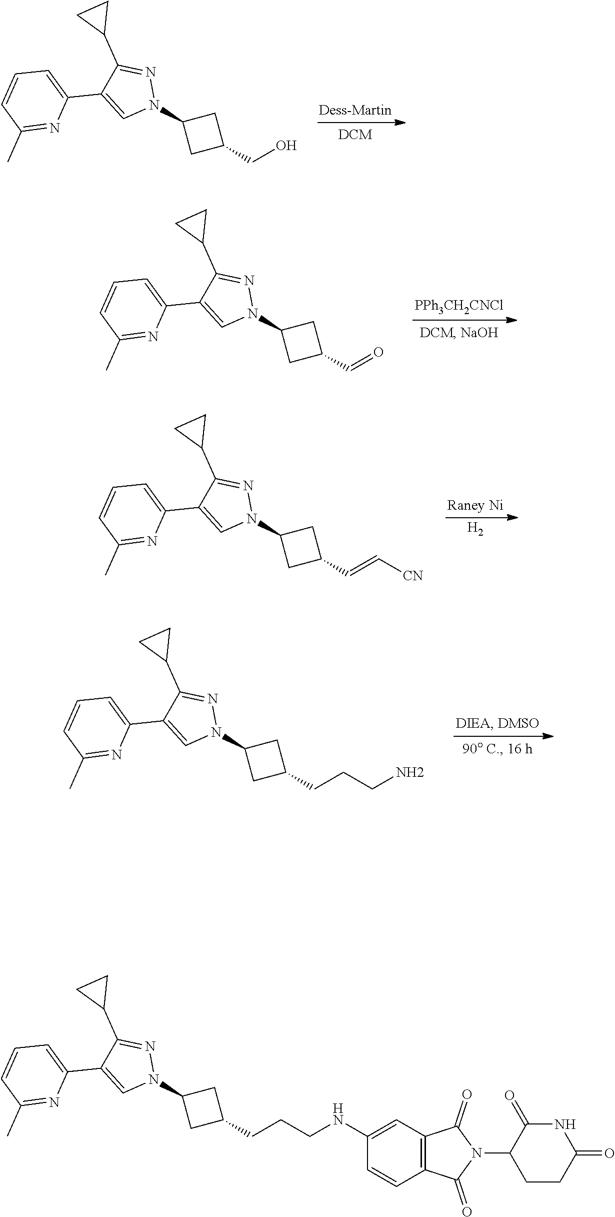 [SMILES: Cc1cccc(-c2cn([C@H]3C[C@H](CO)C3)nc2C2CC2)n1 ; Cc1cccc(-c2cn([C@H]3C[C@H](C=O)C3)nc2C2CC2)n1 ; Cc1cccc(-c2cn([C@H]3C[C@H](/C=C/C#N)C3)nc2C2CC2)n1 ; "H2" ; Cc1cccc(-c2cn([C@H]3C[C@H](CCCN)C3)nc2C2CC2)n1 ; Cc1cccc(-c2cn([C@H]3C[C@H](CCCNc4ccc5c(c4)C(=O)N(C4CCC(=O)NC4=O)C5=O)C3)nc2C2CC2)n1]

GS-592 was synthesized following the similar procedures as described for GS-591 (23 mg) as yellow solid. MS (ESI) m/z=567.3 [M+H]⁺.

### Example 551. 2-(2,6-Dioxopiperidin-3-yl)-5-((3-(trans-3-(4-(7-(4-(2,2,2-trifluoroethyl)piperazin-1-yl)quinoxalin-2-yl)-1H-pyrazol-1-yl)cyclobutyl)propyl)amino)isoindoline-1,3-dione (GS-593)

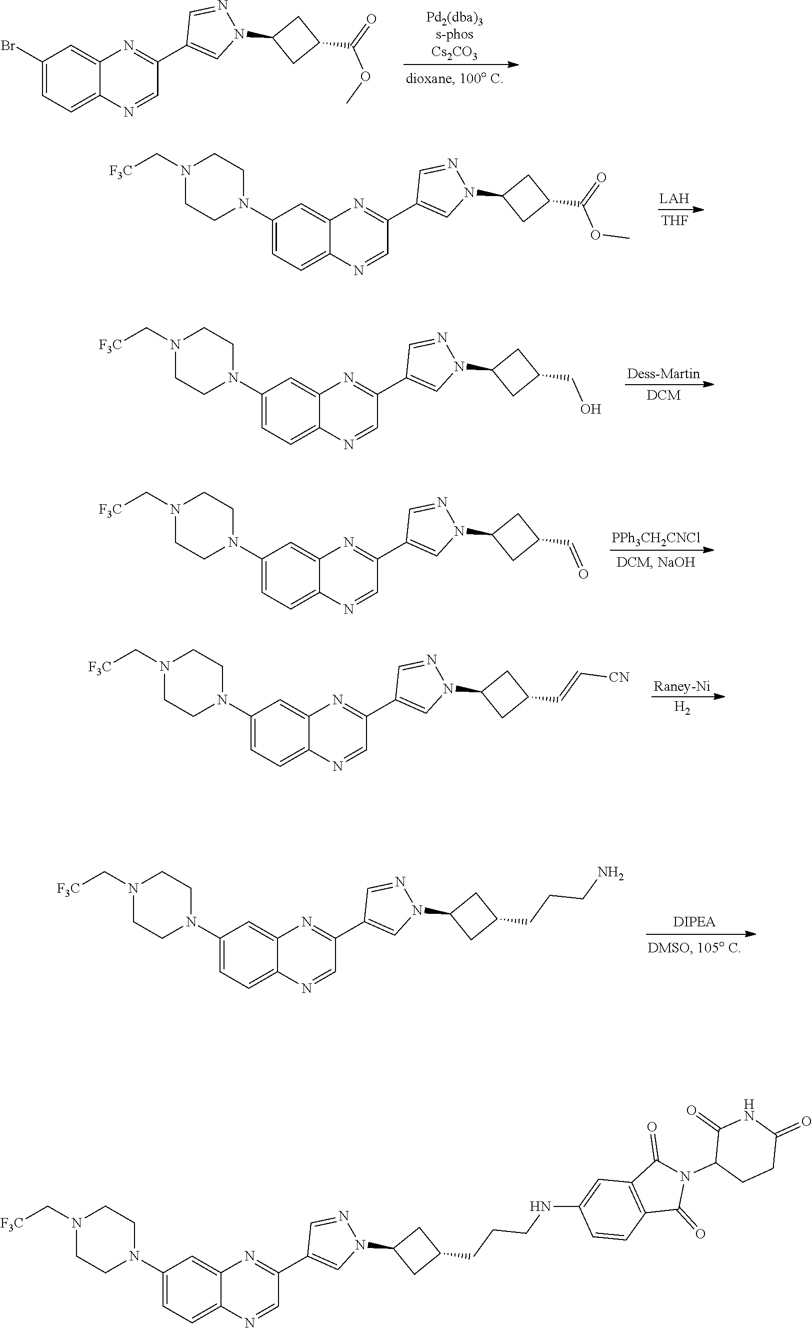

GS-593 was synthesized following the standard procedures for preparing GS-399 and GS-591 (24.9 mg, yield: 10%) as a yellow solid. MS (ESI) m/z=730.7 [M+H]⁺.

### Example 552. 5-((3-(trans-3-(3-Cyclopropyl-4-(7-((1R,4R)-5-methyl-2,5-diazabicyclo[2.2.1]heptan-2-yl)quinoxalin-2-yl)-1H-pyrazol-1-yl)cyclobutyl)propyl)amino)-2-(2,6-dioxopiperidin-3-yl)isoindoline-1,3-dione (GS-594)

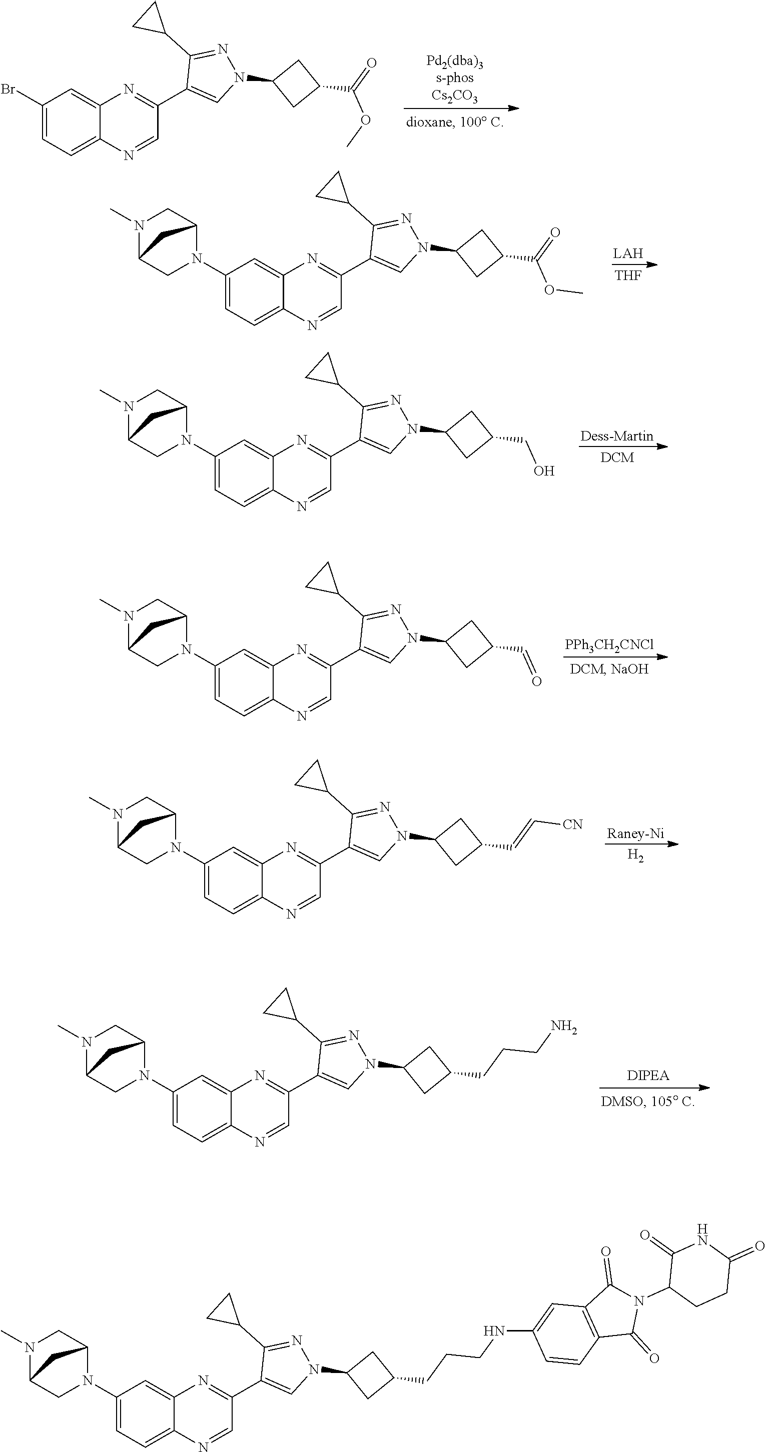

GS-594 was synthesized following the standard procedures for preparing GS-399 and GS-591 (14.5 mg) as a yellow solid. MS (ESI) m/z=714.8 [M+H]⁺.

### Example 553. 5-(((trans-3-(3-Cyclopropyl-4-(pyridin-2-yl)-1H-pyrazol-1-yl)cyclobutyl)methyl)amino)-2-(2,6-dioxopiperidin-3-yl)isoindoline-1,3-dione (GS-595)

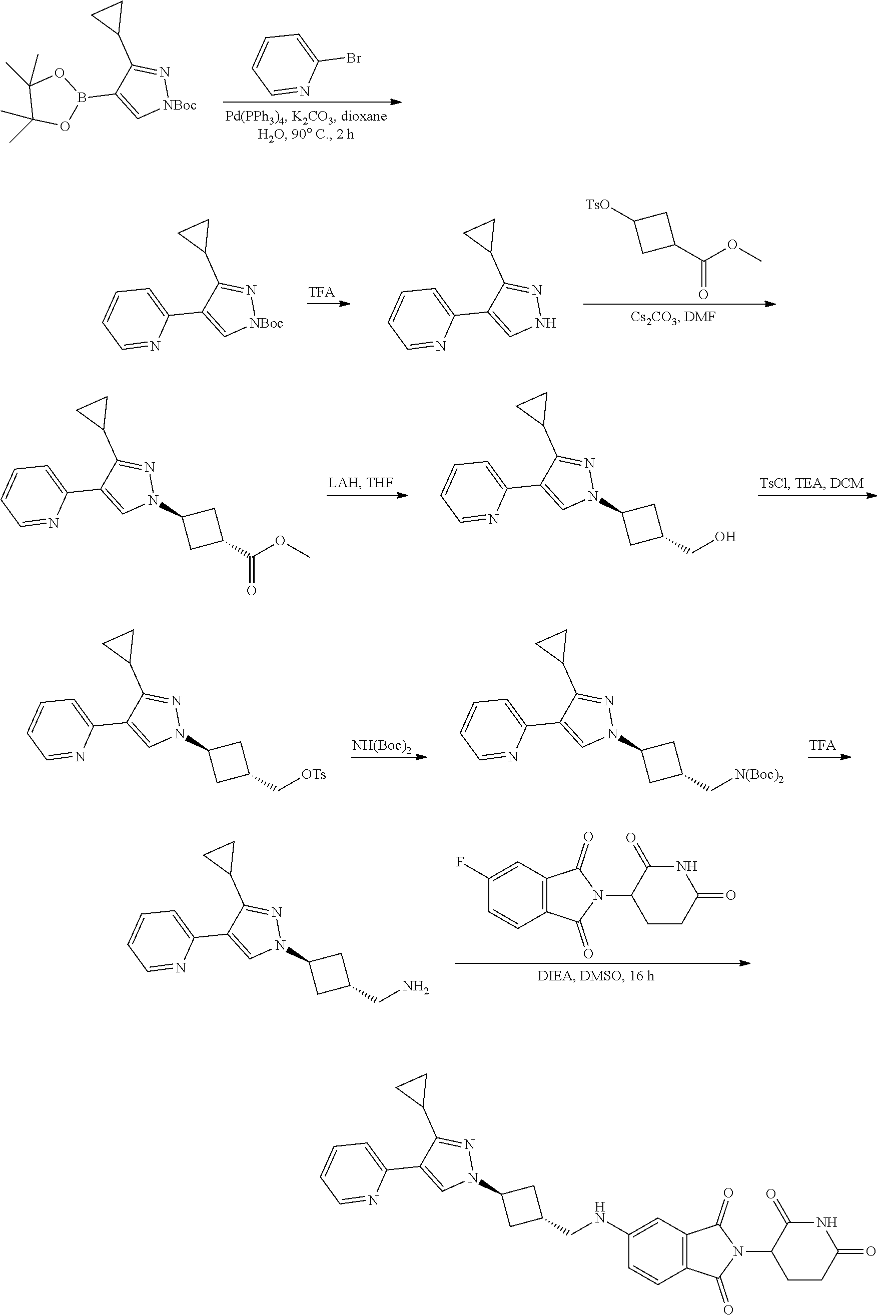

GS-595 was synthesized following the standard procedures for preparing GS-568, GS-440 and GS-591 (3 mg, yield: 10%) as a yellow solid. MS (ESI) m/z=525.4 [M+H]⁺.

### Example 554. 5-(((trans-3-(3-Cyclopropyl-4-(pyrazin-2-yl)-1H-pyrazol-1-yl)cyclobutyl)methyl)amino)-2-(2,6-dioxopiperidin-3-yl)isoindoline-1,3-dione (GS-596)

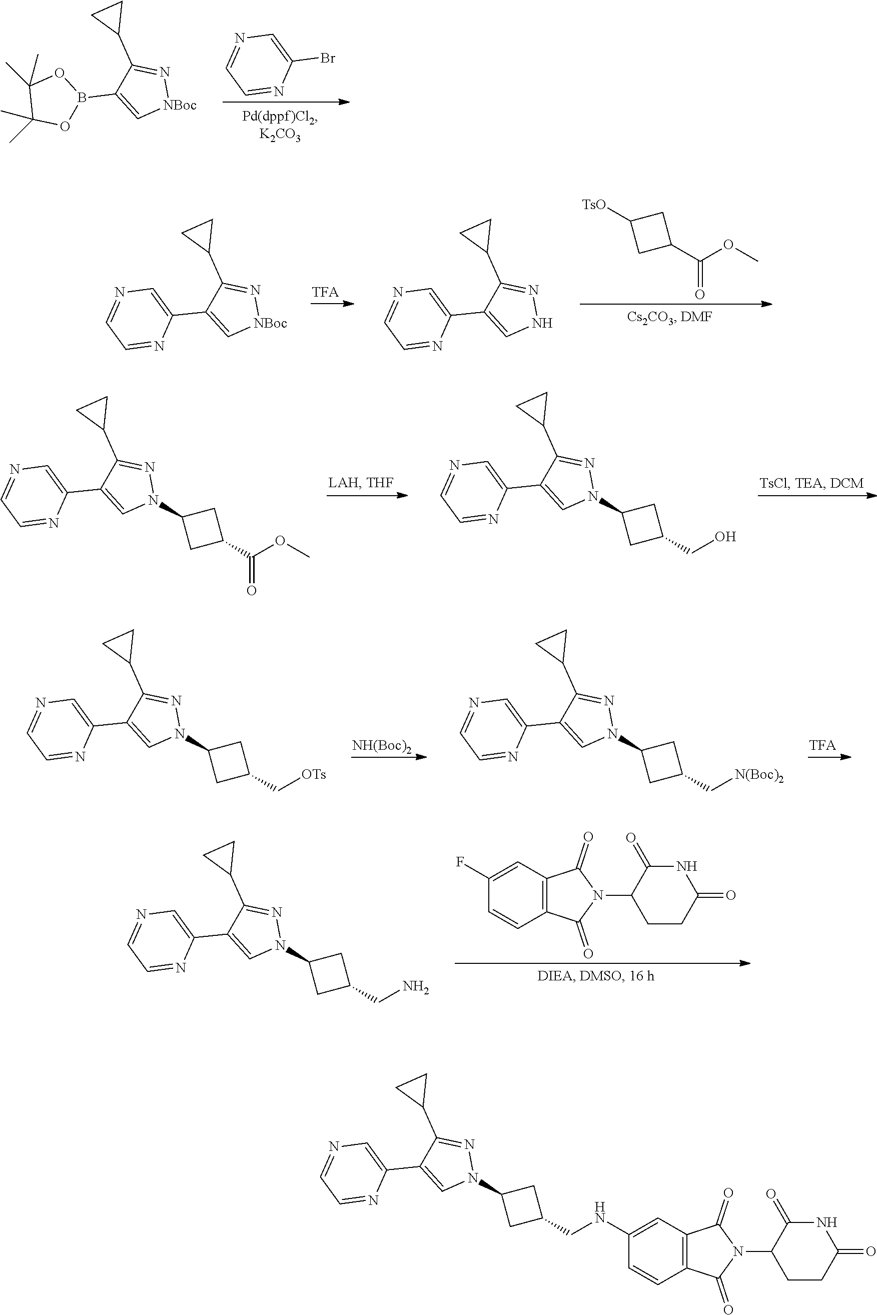

GS-596 was synthesized following the standard procedures for prepraring GS-440 and GS-591 (0.3 mg) as a yellow solid. MS (ESI) m/z=526.3 [M+H]⁺.

### Example 555. 5-(((trans-3-(3-Cyclopropyl-4-(pyridazin-3-yl)-1H-pyrazol-1-yl)cyclobutyl)methyl)amino)-2-(2,6-dioxopiperidin-3-yl)isoindoline-1,3-dione (GS-597)

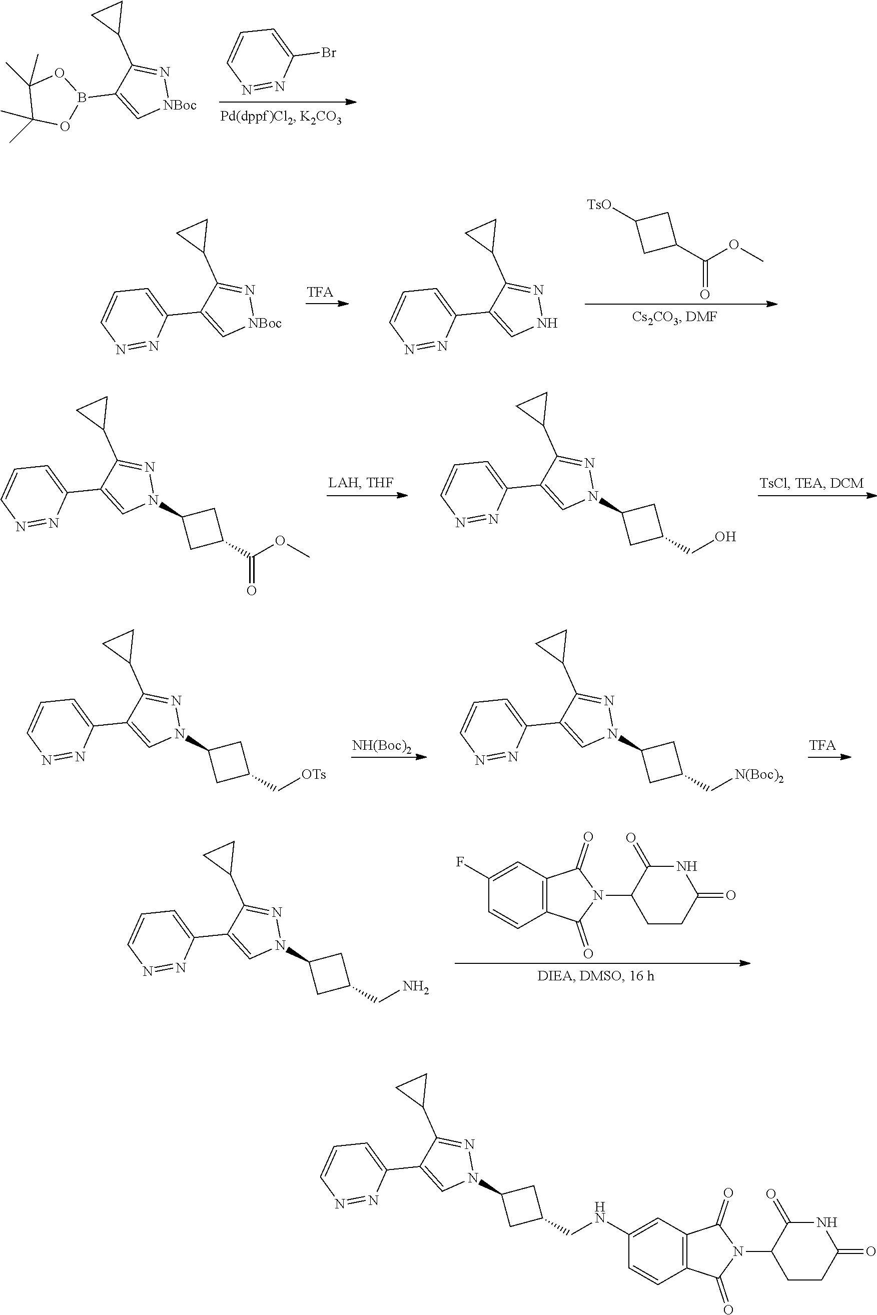

GS-597 was synthesized following the standard procedures for preparing GS-440 and GS-591 (5 mg) as a yellow solid. MS (ESI) m/z=526.6 [M+H]⁺.

### Example 556. 5-(((trans-3-(3-Cyclopropyl-4-phenyl-1H-pyrazol-1-yl)cyclobutyl)methyl)amino)-2-(2,6-dioxopiperidin-3-yl)isoindoline-1,3-dione (GS-598)

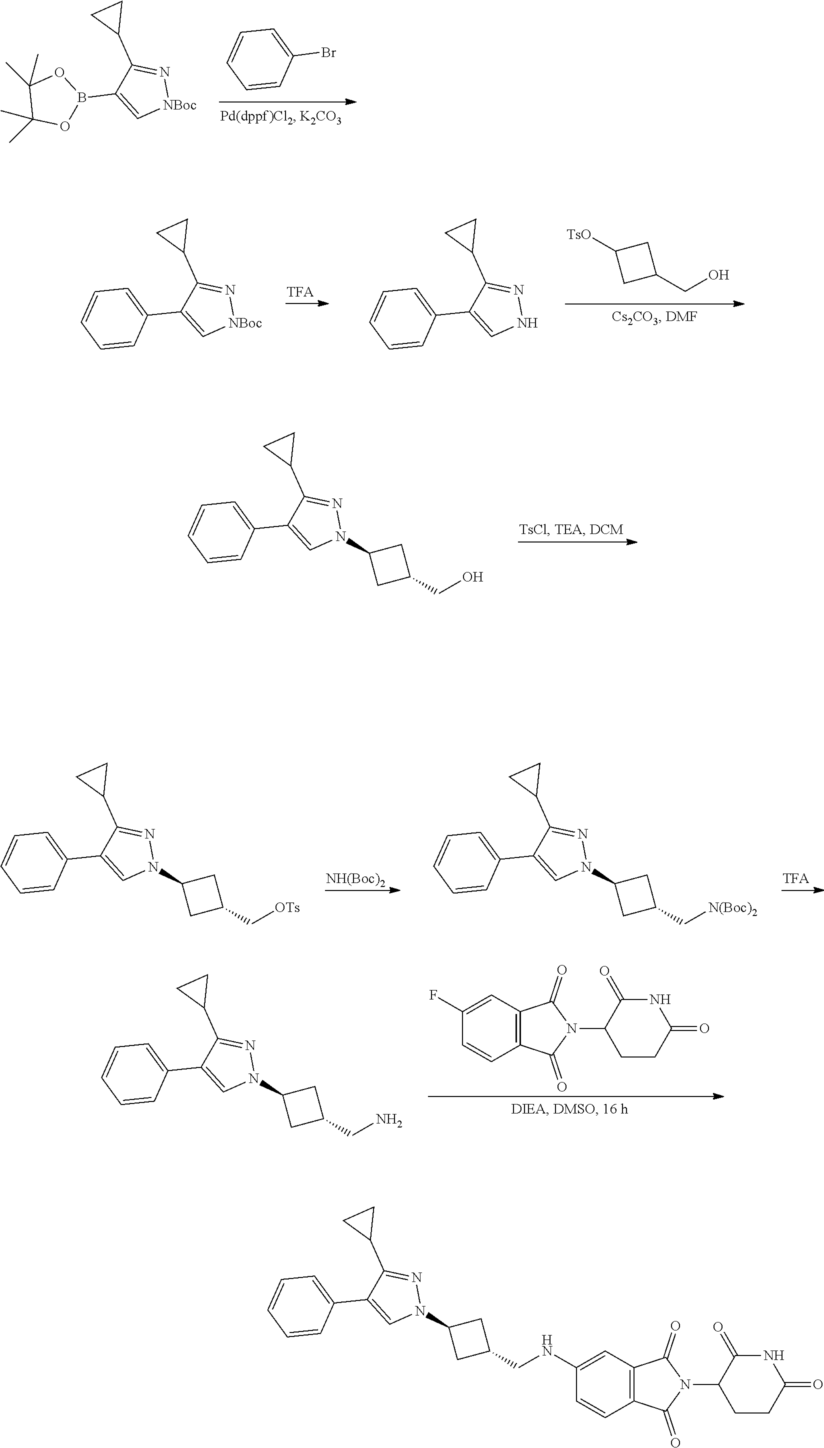

GS-598 was synthesized following the standard procedures for preparing GS-440 and GS-579 (5 mg) as a yellow solid. MS (ESI) m/z=524.5 [M+H]⁺.

### Example 557. 5-(((trans-3-(3-Cyclopropyl-4-(pyrimidin-2-yl)-1H-pyrazol-1-yl)cyclobutyl)methyl)amino)-2-(2,6-dioxopiperidin-3-yl)isoindoline-1,3-dione (GS-599)

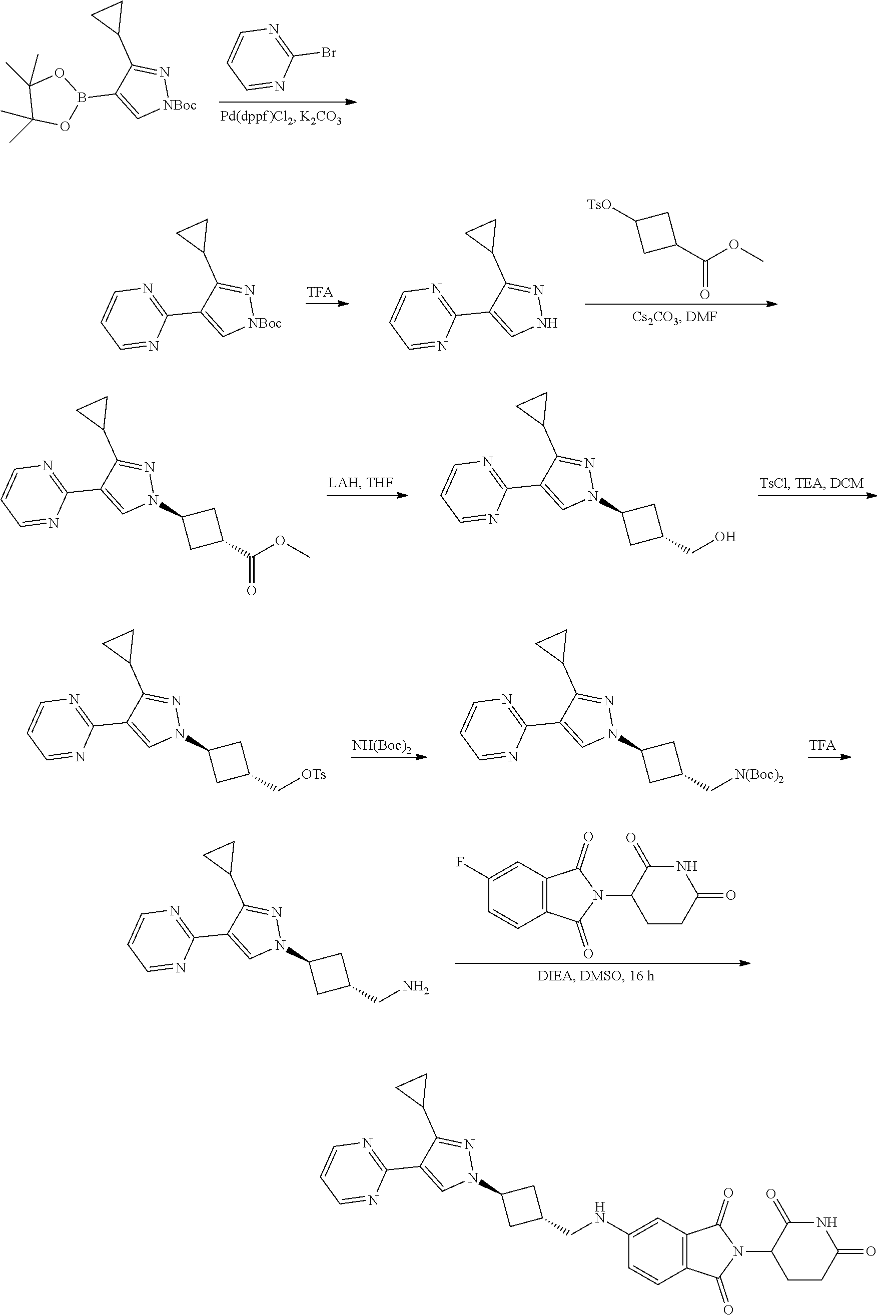

GS-599 was synthesized following the standard procedures or preparing GS-440 an GS-591 (68 mg) as a yellow solid. MS (ESI) m/z=526.4 [M+H]⁺.

### Example 558. 5-((3-(trans-3-(3-Cyclopropyl-4-(quinoxalin-2-yl)-1H-pyrazol-1-yl)cyclobutyl)propyl)amino)-2-(1-methyl-2,6-dioxopiperidin-3-yl)isoindoline-1,3-dione (GS-600)

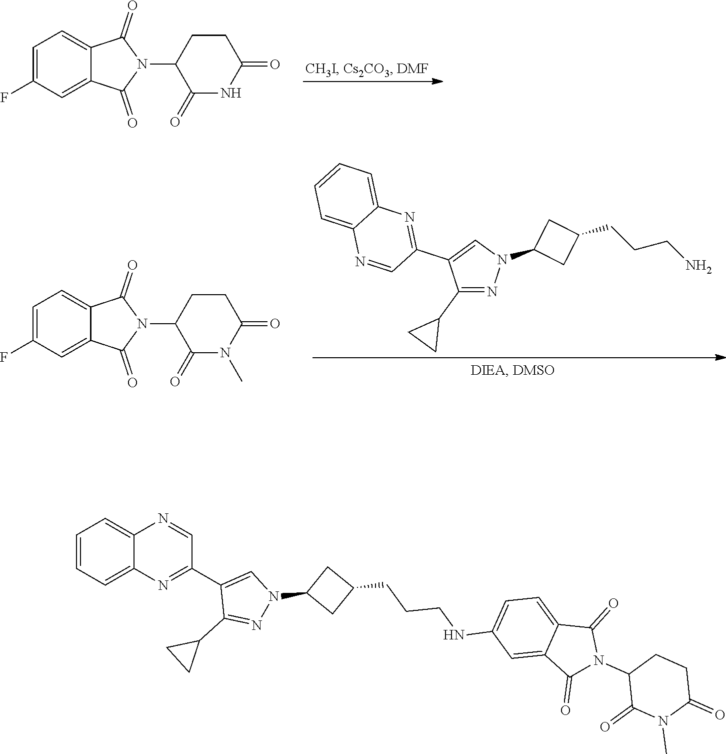 [SMILES: O=C1CCC(N2C(=O)c3ccc(F)cc3C2=O)C(=O)N1 ; CN1C(=O)CCC(N2C(=O)c3ccc(F)cc3C2=O)C1=O ; NCCC[C@H]1C[C@H](n2cc(-c3cnc4ccccc4n3)c(C3CC3)n2)C1 ; CN1C(=O)CCC(N2C(=O)c3ccc(NCCC[C@H]4C[C@H](n5cc(-c6cnc7ccccc7n6)c(C6CC6)n5)C4)cc3C2=O)C1=O]

Step 1. Synthesis of 5-fluoro-2-(1-methyl-2,6-dioxopiperidin-3-yl)isoindoline-1,3-dione

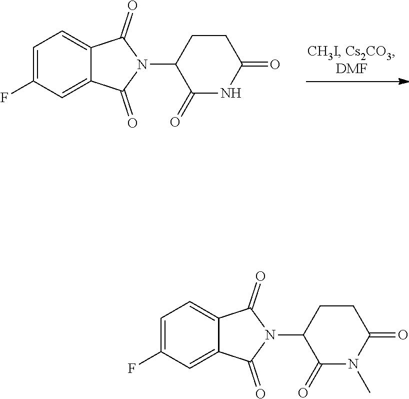 [SMILES: O=C1CCC(N2C(=O)c3ccc(F)cc3C2=O)C(=O)N1 ; CN1C(=O)CCC(N2C(=O)c3ccc(F)cc3C2=O)C1=O]

To a solution of 2-(2,6-dioxo-3-piperidyl)-5-fluoro-isoindoline-1,3-dione (1 g, 3.62 mmol) in DMF (10 mL) were added iodomethane (1.03 g, 7.24 mmol) and Cs₂CO₃ (2.35 g, 7.24 mmol). After the mixture was stirred at 90° C. for 1 h, it was quenched with water (50 mL) and extracted with EtOAc (15 mL×3). The combined organic phase was washed with brine, dried over anhydrous sodium sulfate, filtered, and concentrated to provide the desired crude compound as a yellow solid. MS (ESI) m/z=291.3 [M+H]⁺.

Step 2. Synthesis of 5-((3-(trans-3-(3-cyclopropyl-4-(quinoxalin-2-yl)-1H-pyrazol-1-yl)cyclobutyl)propyl)amino)-2-(1-methyl-2,6-dioxopiperidin-3-yl)isoindoline-1,3-dione

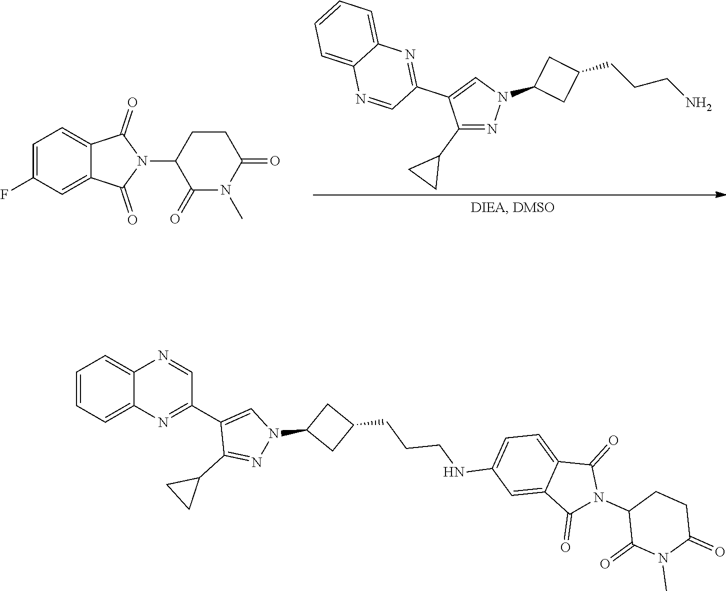 [SMILES: CN1C(=O)CCC(N2C(=O)c3ccc(F)cc3C2=O)C1=O ; NCCC[C@H]1C[C@H](n2cc(-c3cnc4ccccc4n3)c(C3CC3)n2)C1 ; CN1C(=O)CCC(N2C(=O)c3ccc(NCCC[C@H]4C[C@H](n5cc(-c6cnc7ccccc7n6)c(C6CC6)n5)C4)cc3C2=O)C1=O]

To a solution of 3-(trans-3-(3-cyclopropyl-4-(quinoxalin-2-yl)-1H-pyrazol-1-yl)cyclobutyl)propan-1-amine (400 mg, 1.15 mmol) in DMSO (6 mL) were added 5-fluoro-2-(1-methyl-2,6-dioxo-3-piperidyl)isoindoline-1,3-dione (367.55 mg, 1.27 mmol) and DIPEA (297.57 mg, 2.30 mmol). The mixture was stirred at 115° C. for 16 h. After cooling down to rt, the mixture was purified by reverse phase chromatography to provide the title compound (152 mg, yield: 21%) as a yellow solid. MS (ESI) m/z=618.5 [M+H]⁺.

### Example 559. 5-(((trans-3-(3-Cyclopropyl-4-(pyrimidin-4-yl)-1H-pyrazol-1-yl)cyclobutyl)methyl)amino)-2-(2,6-dioxopiperidin-3-yl)isoindoline-1,3-dione (GS-601)

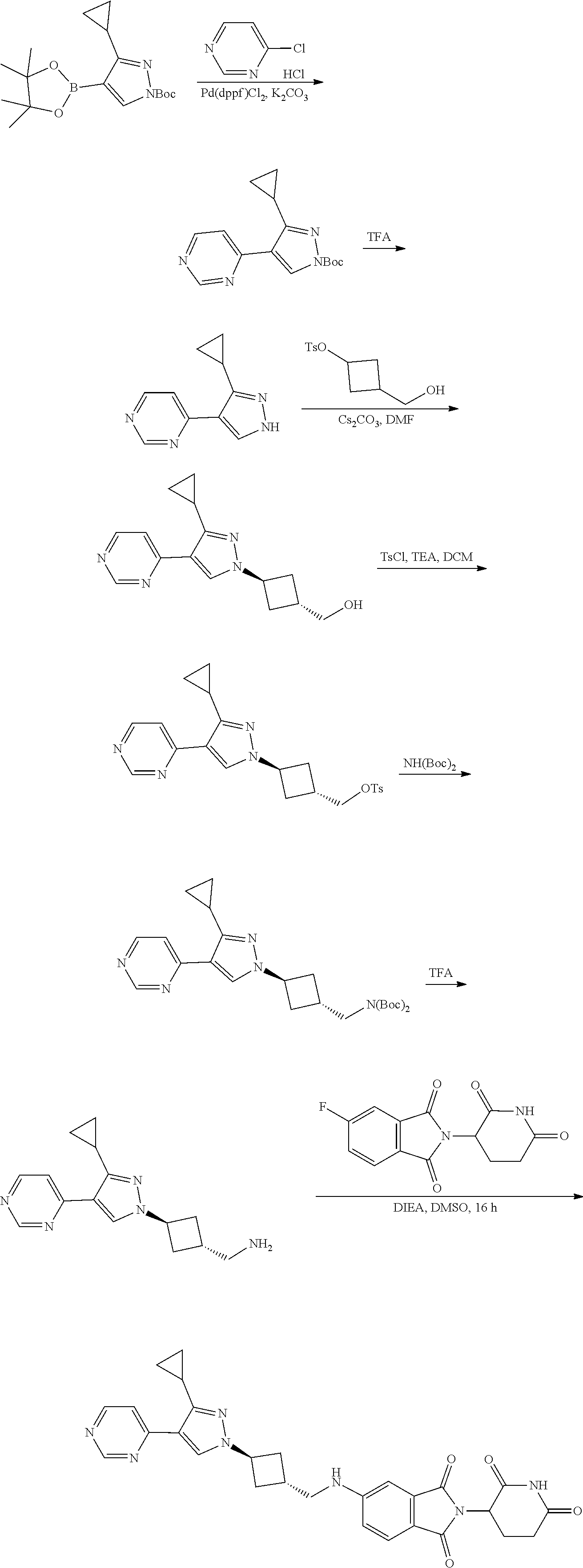

GS-601 was synthesized following the standard procedures for preparing GS-440 and GS-579 (56 mg) as a yellow solid. MS (ESI) m/z=526.6 [M+H]⁺.

### Example 560. 2-(2,6-Dioxopiperidin-3-yl)-5-((3-(trans-3-(4-(3-methylpyridin-2-yl)-1H-pyrazol-1-yl)cyclobutyl)propyl)amino)isoindoline-1,3-dione (GS-602)

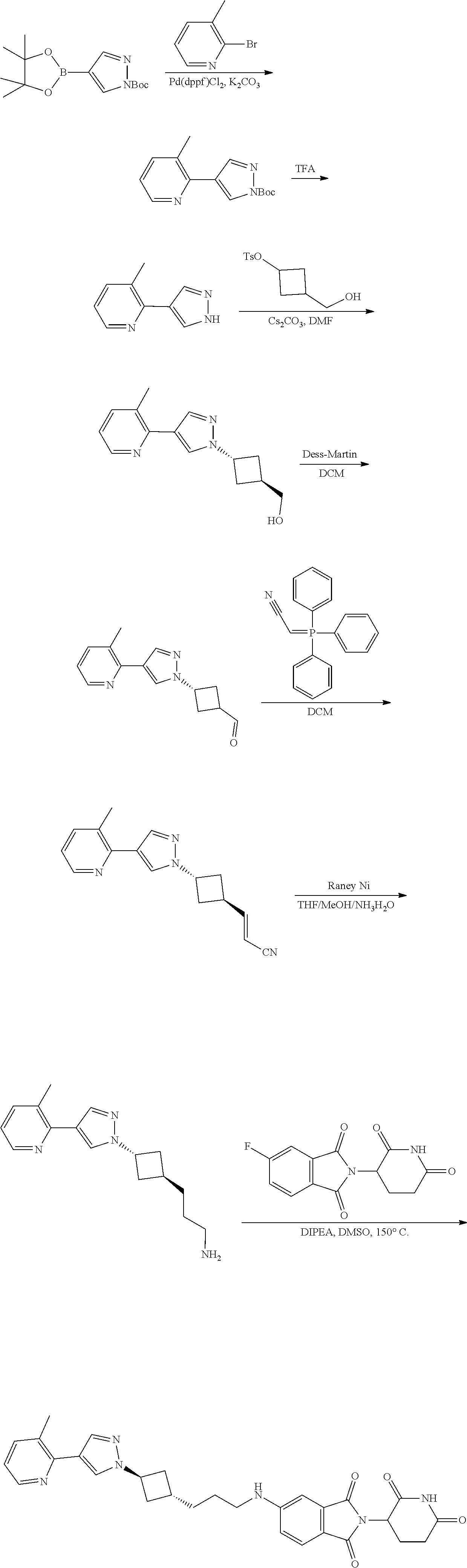

Step 1 to 2. Synthesis of 3-methyl-2-(1H-pyrazol-4-yl)pyridine

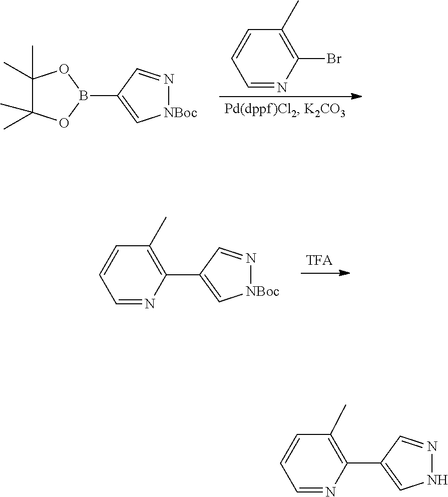

The title compound was synthesized following the standard procedures for preparing GS-440 (560 mg, yield: 91%) as a colorless oil. MS (ESI) m/z=160.2 [M+H]⁺.

Step 3. Synthesis of (trans-3-(4-(3-methylpyridin-2-yl)-1H-pyrazol-1-yl)cyclobutyl)methanol

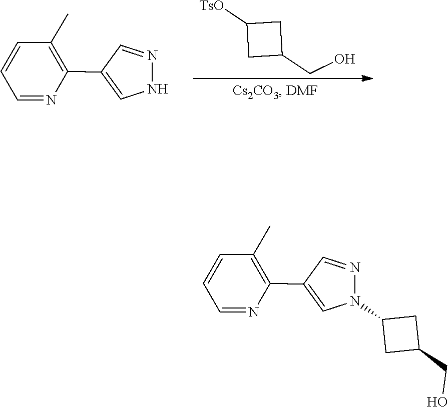 [SMILES: Cc1cccnc1-c1cn[nH]c1 ; Cc1ccc(S(=O)(=O)OC2CC(CO)C2)cc1 ; Cc1cccnc1-c1cnn([C@H]2C[C@H](CO)C2)c1]

To a solution of 3-methyl-2-(1H-pyrazol-4-yl)pyridine (700 mg, 4.40 mmol) in DMSO (25 mL) were added 3-(hydroxymethyl)cyclobutyl 4-methylbenzenesulfonate (1.35 g, 5.28 mmol) and Cs₂CO₃ (2.86 g, 8.79 mmol). After the mixture was stirred at 100° C. for 2 h, it was purified by reverse phase chromatography to provide the title compound (600 mg, yield: 56%) as yellow oil. MS (ESI) m/z=244.3 [M+H]⁺.

Step 4. Synthesis of trans-3-(4-(3-methylpyridin-2-yl)-1H-pyrazol-1-yl)cyclobutane-1-carbaldehyde

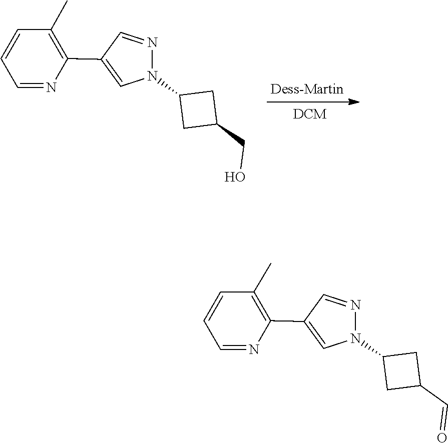

To a solution of (trans-3-(4-(3-methylpyridin-2-yl)-1H-pyrazol-1-yl)cyclobutyl)methanol (600 mg, 2.47 mmol) in DCM (20 mL) was added Dess-Martin reagent (2.09 g, 4.93 mmol). After the mixture was stirred at rt for 1 h, it was filtered. The filtrate was concentrated to provide the crude desired product (500 mg, yield: 84%) as a yellow oil. MS (ESI) m/z=242.3 [M+H]⁺.

Step 5. Synthesis of 3-(trans-3-(4-(3-methylpyridin-2-yl)-1H-pyrazol-1-yl)cyclobutyl)acrylonitrile

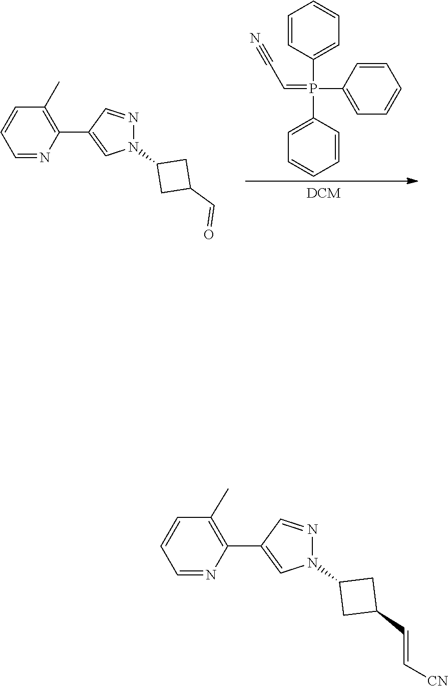

To a solution of trans-3-(4-(3-methylpyridin-2-yl)-1H-pyrazol-1-yl)cyclobutane-1-carbaldehyde (500 mg, 2.07 mmol) in DCM (10 mL) was added (triphenylphosphoranylidene)acetonitrile (686.84 mg, 2.28 mmol). After the mixture was stirred at rt for 20 min, it was concentrated under reduced pressure. The resulting residue was purified by reverse phase chromatography to provide the title compound (410 mg, yield: 75%) as a yellow oil. MS (ESI) m/z=265.3 [M+H]⁺.

Step 6. Synthesis of 3-(trans-3-(4-(3-methylpyridin-2-yl)-1H-pyrazol-1-yl)cyclobutyl)propan-1-amine

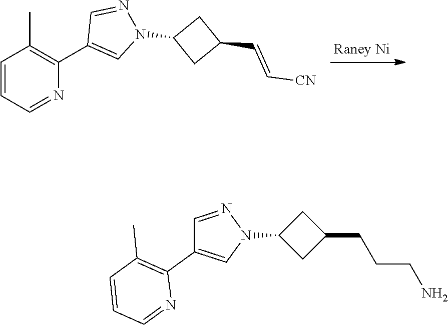

To a solution of 3-(trans-3-(4-(3-methylpyridin-2-yl)-1H-pyrazol-1-yl)cyclobutyl)acrylonitrile (410 mg, 1.55 mmol) in methanol (5 mL) were added THF (10 mL) and Raney Ni. The mixture was purged with hydrogen and stirred at rt for 4 h under hydrogen balloon. The mixture was filtered through Celite. The filtrate was concentrated and purified by reverse phase chromatography to provide the title compound (320 mg, yield: 76%) as a yellow oil. MS (ESI) m/z=271.3 [M+H]⁺.

Step 7. Synthesis of 2-(2,6-dioxopiperidin-3-yl)-5-((3-(trans-3-(4-(3-methylpyridin-2-yl)-1H-pyrazol-1-yl)cyclobutyl)propyl)amino)isoindoline-1,3-dione

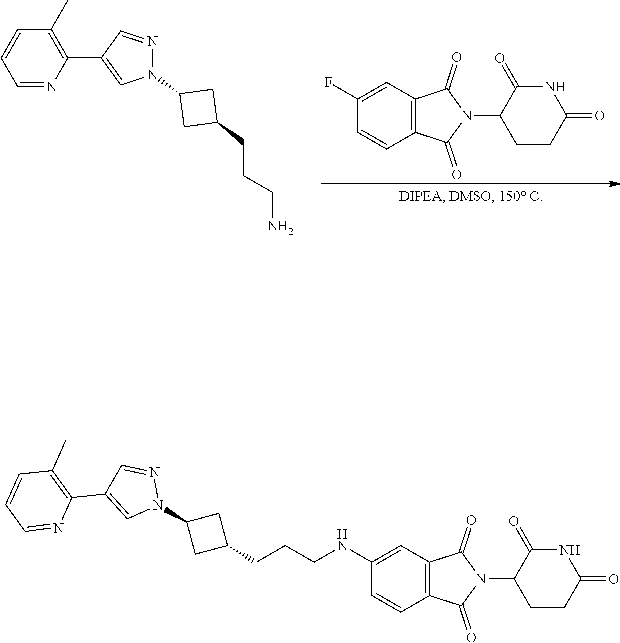 [SMILES: Cc1cccnc1-c1cnn([C@H]2C[C@H](CCCN)C2)c1 ; O=C1CCC(N2C(=O)c3ccc(F)cc3C2=O)C(=O)N1 ; Cc1cccnc1-c1cnn([C@H]2C[C@H](CCCNc3ccc4c(c3)C(=O)N(C3CCC(=O)NC3=O)C4=O)C2)c1]

To a solution of 3-(trans-3-(4-(3-methylpyridin-2-yl)-1H-pyrazol-1-yl)cyclobutyl)propan-1-amine (70 mg, 258.90 μmol) in DMSO (2 mL) were added 2-(2,6-dioxo-3-piperidyl)-5-fluoro-isoindoline-1,3-dione (78.67 mg, 284.79 μmol) and DIPEA (66.92 mg, 517.81 μmol). The mixture was stirred at 150° C. for 2 h. After cooling down to rt, the mixture was purified by reverse phase chromatography to provide the title compound (50 mg, yield: 37%) as a yellow solid. MS (ESI) m/z=527.5 [M+H]⁺.

### Example 561. 2-(2,6-Dioxopiperidin-3-yl)-5-((3-(trans-3-(4-(4-methylpyridin-2-yl)-1H-pyrazol-1-yl)cyclobutyl)propyl)amino)isoindoline-1,3-dione (GS-603)

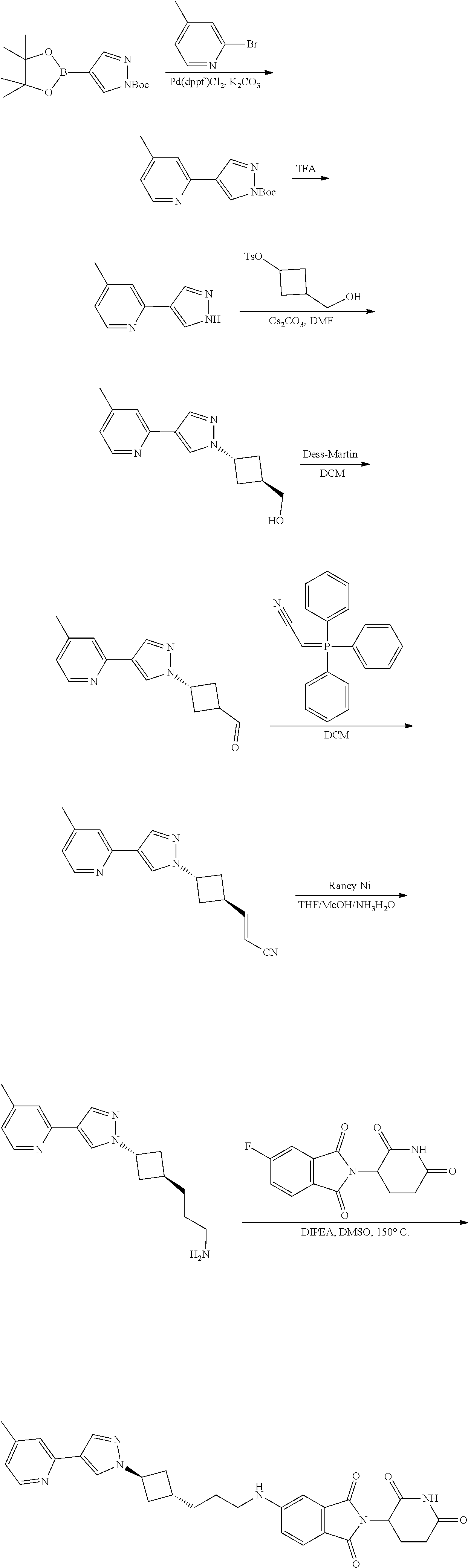

GS-603 was synthesized following the standard procedures for preparing GS-602 (50 mg) as a yellow solid. MS (ESI) m/z=527.5 [M+H]⁺.

### Example 562. 2-(2,6-Dioxopiperidin-3-yl)-5-((3-(trans-3-(4-(3-methoxypyridin-2-yl)-1H-pyrazol-1-yl)cyclobutyl)propyl)amino)isoindoline-1,3-dione (GS-494)

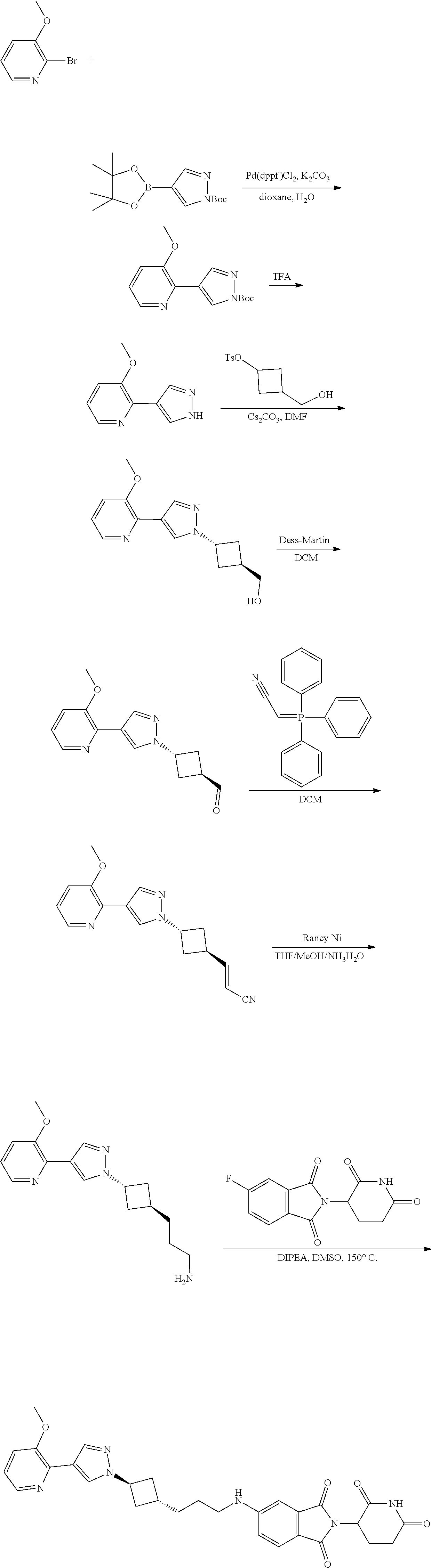

GS-494 was synthesized following the standard procedures for preparing GS-602 (30 mg) as a yellow solid. MS (ESI) m/z=543.5 [M+H]⁺.

### Example 563. 2-(2,6-Dioxopiperidin-3-yl)-5-((3-(trans-3-(4-(4-methoxypyridin-2-yl)-1H-pyrazol-1-yl)cyclobutyl)propyl)amino)isoindoline-1,3-dione (GS-341)

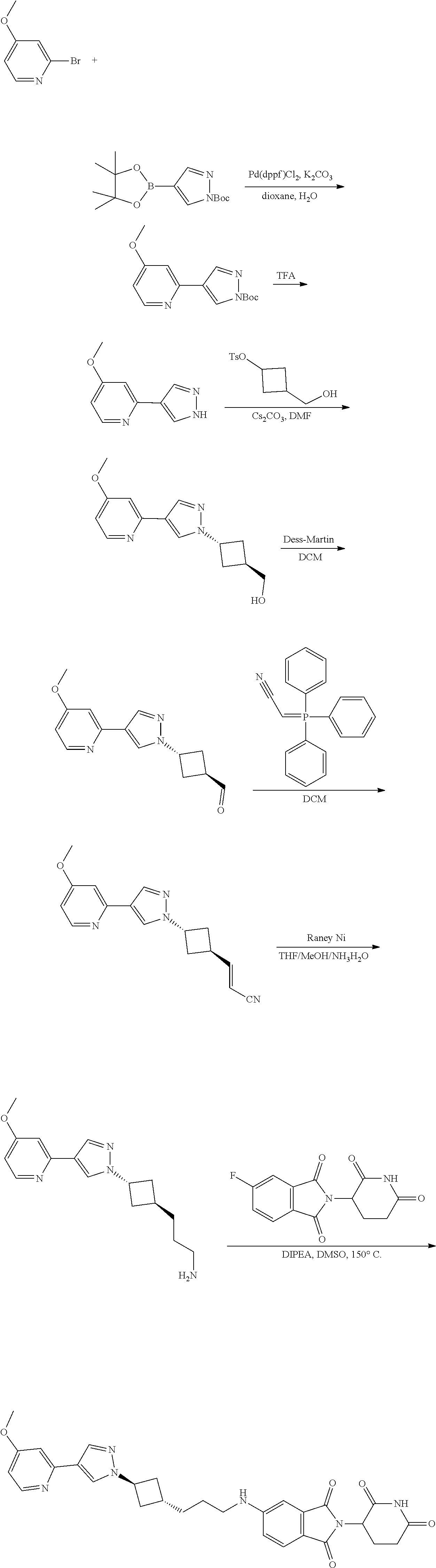

GS-341 was synthesized following the standard procedures for preparing GS-602 (50 mg) as a yellow solid. MS (ESI) m/z=543.5 [M+H]⁺.

### Example 564. 5-((3-(trans-3-(4-(4-Chloropyridin-2-yl)-1H-pyrazol-1-yl)cyclobutyl)propyl)amino)-2-(2,6-dioxopiperidin-3-yl)isoindoline-1,3-dione (GS-328)

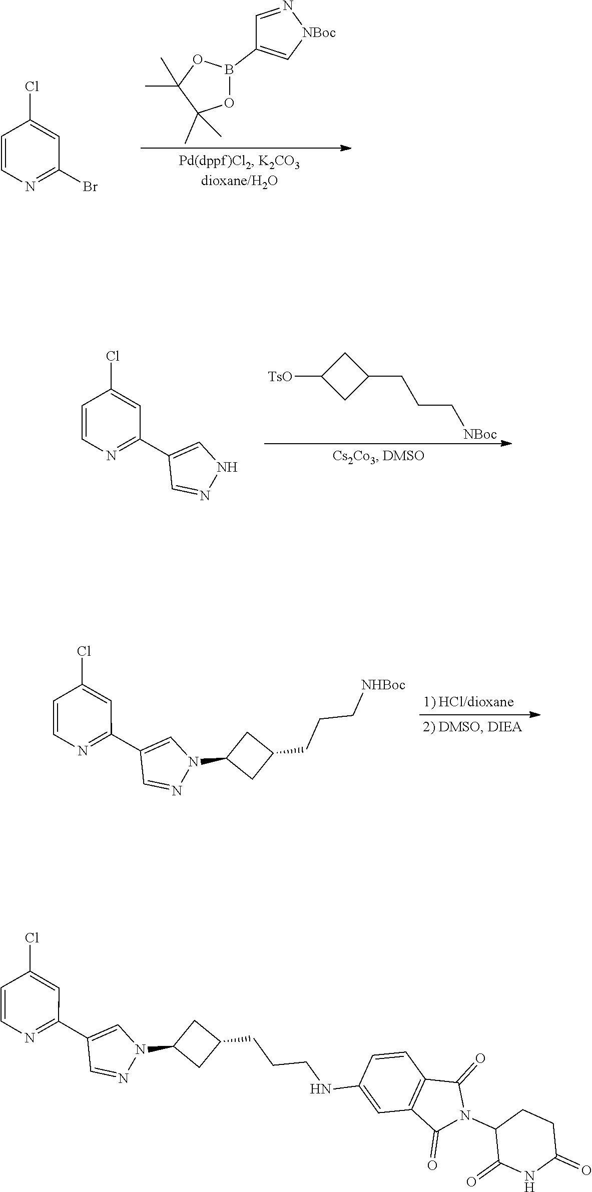

GS-328 was synthesized following the similar procedures as described for GS-311 (10 mg) as yellow solid. MS (ESI) m/z=547.7 [M+H]⁺.

### Example 565. 5-((3-(trans-3-(4-(3-Chloropyridin-2-yl)-1H-pyrazol-1-yl)cyclobutyl)propyl)amino)-2-(2,6-dioxopiperidin-3-yl)isoindoline-1,3-dione (GS-311)

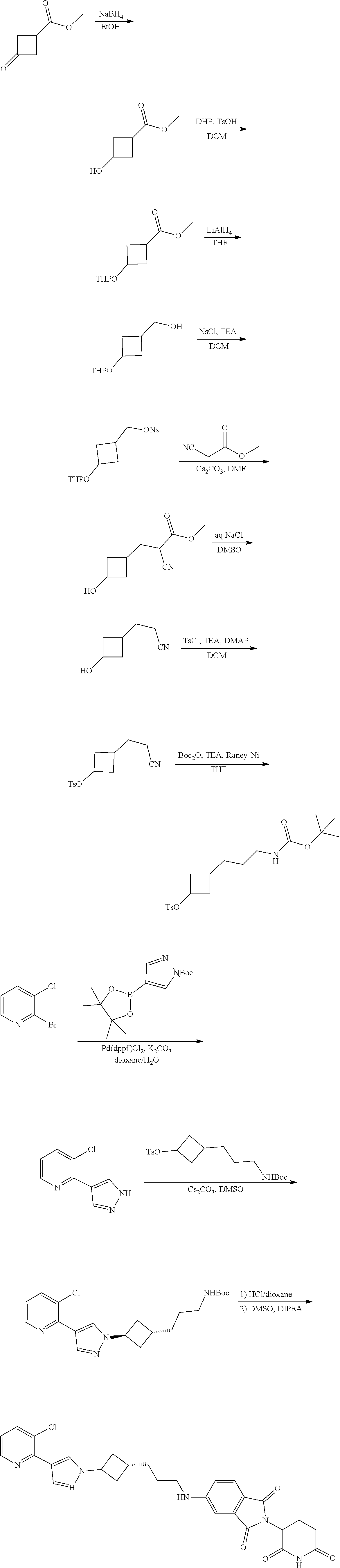

Step 1. Synthesis of methyl 3-hydroxycyclobutane-1-carboxylate

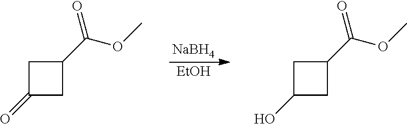

To a solution of methyl 3-oxocyclobutanecarboxylate (30 g, 234.15 mmol) in ethanol (200 mL) was added NaBH₄ (13.35 g, 351.22 mmol) in portions at −10° C. The mixture was stirred at this temperature for 30 min. The mixture was quenched with aq. NH₄Cl solution (10%, 200 mL). The mixture was concentrated under vacuum to remove most of ethanol. The resulting residue was extracted with ethyl acetate (300 mL×2). The combined organic layers were washed with brine, dried over anhydrous sodium sulfate, filtered, and concentrated under reduced pressure to provide the crude compound (25 g, yield: 82%) as a colorless oil.

Step 2. Synthesis of methyl 3-((tetrahydro-2H-pyran-2-yl)oxy)cyclobutane-1-carboxylate

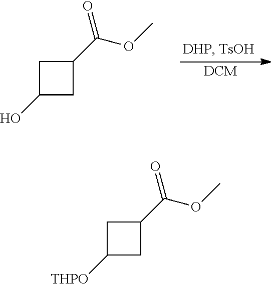

To a solution of methyl 3-hydroxycyclobutane-1-carboxylate (25 g, 192.10 mmol) in DCM (200 mL) were added TsOH (0.25 g) and 3,4-dihydro-2H-pyran (17.77 g, 211.31 mmol) dropwise. The mixture was stirred at 25° C. for 0.5 h. The mixture was washed with aq. Na₂CO₃ solution (5%, 50 mL). The organic layer was dried over anhydrous sodium sulfate, filtered, and concentrated under reduced pressure to provide the crude compound (41.1 g, yield: 99.9%) as a yellow oil.

Step 3. Synthesis of (3-((tetrahydro-2H-pyran-2-yl)oxy)cyclobutyl)methanol

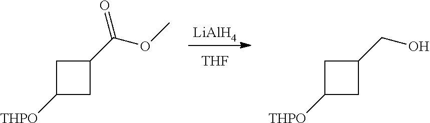

To a suspension of LiAlH₄ (8.02 g, 211.01 mmol) in THF (200 mL) was added a solution of methyl 3-((tetrahydro-2H-pyran-2-yl)oxy)cyclobutane-1-carboxylate (41.1 g, 191.83 mmol) in THF (200 mL). After the mixture was stirred at 25° C. for 0.5 h, it was quenched with water (15 mL) slowly. The obtained mixture was filtered. The filter cake was washed with THF (300 mL). The filtrate was concentrated under reduced pressure to provide the crude compound (34.3 g, yield: 96%) as a yellow oil.

Step 4. Synthesis of (3-((tetrahydro-2H-pyran-2-yl)oxy)cyclobutyl)methyl 4-nitrobenzenesulfonate

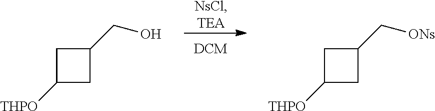

To a solution of (3-((tetrahydro-2H-pyran-2-yl)oxy)cyclobutyl)methanol (34.3 g, 184.16 mmol) in DCM (300 mL) was added TEA (56.30 g, 552.49 mmol). 4-Nitrobenzenesulfonyl chloride (44.90 g, 202.58 mmol) was added in portions at 25° C. The mixture was stirred at this temperature for 0.5 h. The mixture was washed with water (100 mL) and aq. Na₂CO₃ solution (10%, 100 mL). The organic layer was concentrated under reduced pressure to remove most of DCM. To the resulting residue was added EtOAc (200 mL). The mixture was concentrated under reduced pressure. Heptane (300 mL) was added. After the suspension was stirred for 1 h, it was filtered. The filter cake was dried in vacuo to provide the crude compound (53 g, yield: 77%) as a yellow solid.

Step 5. Synthesis of methyl 2-cyano-3-(3-hydroxycyclobutyl)propanoate

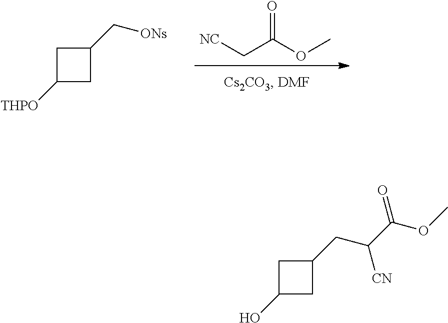

To a solution of methyl 2-cyanoacetate (72.03 g, 726.97 mmol) in DMF (200 mL) was added Cs₂CO₃ (47.37 g, 145.39 mmol) at 60° C. The mixture was stirred at the same temperature for 0.5 h. (3-((Tetrahydro-2H-pyran-2-yl)oxy)cyclobutyl)methyl 4-nitrobenzenesulfonate (27.00 g, 72.70 mmol) was added at 60° C. The mixture was stirred at this temperature for 18 h. After cooling down to rt, the reaction was quenched with water (300 mL) and extracted with EtOAc (100 mL×3). The combined organic layer was dried over anhydrous sodium sulfate, filtered, and concentrated under reduced pressure. To the resulting residue was added THF (100 mL) and aq. HCl solution (5%, 50 mL). After the mixture was stirred for 2 h, it was extracted with EtOAc (100 ml). The organic layer was concentrated under reduced pressure. The resulting residue was purified by silica gel chromatography (petroleum ether/EtOAc=100:1 to 1:1) to provide the title compound (4 g, yield: 30%) as a yellow oil.

Step 6. Synthesis of 3-(3-hydroxycyclobutyl)propanenitrile

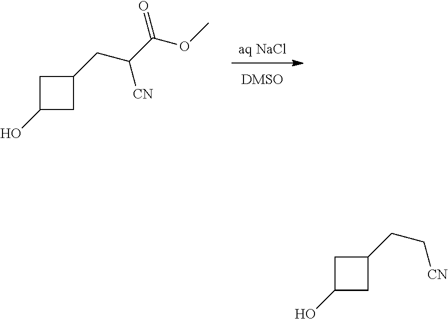

A solution of methyl 2-cyano-3-(3-hydroxycyclobutyl)propanoate (4 g, 21.83 mmol) in brine (30 mL) and DMSO (30 mL) was stirred at 165° C. for 48 h. After cooling down to rt, water (30 mL) was added, and the mixture was extracted with EtOAc (50 mL×2). The combined organic layer was dried over anhydrous sodium sulfate, filtered, and concentrated under reduced pressure to provide the title compound (3.9 g, yield: 99.9%) as a yellow oil.

Step 7. Synthesis of 3-(2-cyanoethyl)cyclobutyl 4-methylbenzenesulfonate

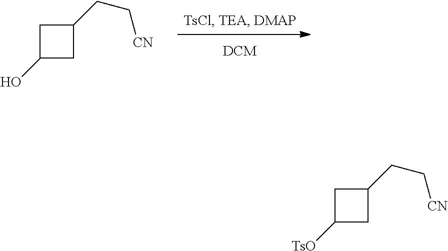

To a solution of 3-(3-hydroxycyclobutyl)propanenitrile (3 g, 23.97 mmol) in DCM (100 mL) were added TEA (7.28 g, 71.90 mmol), DMAP (0.1 g, 818.55 μmol) and 4-methylbenzenesulfonyl chloride (5.03 g, 26.36 mmol). After the mixture was stirred at 25° C. for 4 h, it was concentrated under reduced pressure. The resulting residue was purified by silica gel chromatography (petroleum ether/EtOAc=100:1 to 3:1) to provide the title compound (2.3 g, yield: 34%) as a yellow oil. MS (ESI) m/z=280.15 [M+H]⁺.

Step 8. Synthesis of 3-(3-((tert-butoxycarbonyl)amino)propyl)cyclobutyl 4-methylbenzenesulfonate

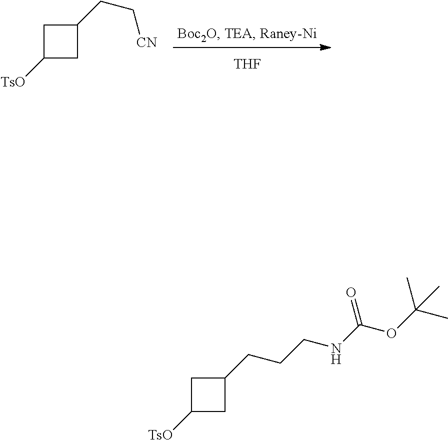 [SMILES: Cc1ccc(S(=O)(=O)OC2CC(CCC#N)C2)cc1 ; "Boc2O, TEA, Raney-Ni" ; Cc1ccc(S(=O)(=O)OC2CC(CCCNC(=O)OC(C)(C)C)C2)cc1 ; "THF"]

To a solution of 3-(2-cyanoethyl)cyclobutyl 4-methylbenzenesulfonate (2.3 g, 8.23 mmol) in THF (100 mL) were added TEA (1.67 g, 16.47 mmol), tert-butoxycarbonyl tert-butyl carbonate (2.34 g, 10.70 mmol) and Raney-Ni (1 g, 17.04 mmol). The mixture was purged with hydrogen and stirred at rt under 15 psi of H₂ for 20 h. The mixture was filtered through Celite and the filtrate was concentrated under reduced pressure. The resulting residue was purified by silica gel chromatography (petroleum ether/EtOAc=100:1 to 5:1) to provide the title compound (3 g, yield: 95%) as a colorless oil. MS (ESI) m/z=284.19 [M−100+H]⁺.

Step 9. Synthesis of 3-chloro-2-(1H-pyrazol-4-yl)pyridine

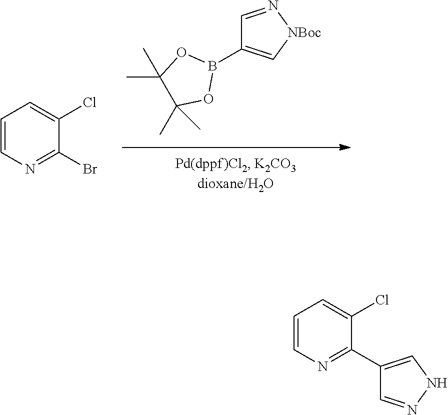

A mixture of 2-bromo-3-chloro-pyridine (1.0 g, 5.20 mmol), tert-butyl 4-(4,4,5,5-tetramethyl-1,3,2-dioxaborolan-2-yl)pyrazole-1-carboxylate (2.5 g, 7.65 mmol), K₂CO₃ (1.43 g, 10.33 mmol) and Pd(dppf)Cl₂ (100 mg) in dioxane (10 mL) and water (1 mL) was heated at reflux under N₂ for 1.5 h. The mixture was concentrated under reduced pressure. To the resulting residue were added MeOH (10 mL) and aq. NaOH solution (30%, 2 eq.). The mixture was heated at 70° C. for 30 min. After cooling down to rt, the mixtue was concentrated under reduced pressure. The resulting residue was diluted with water (60 mL). The precipitate was collected and dried to provide the crude compound (900 mg, yield: 81%) as a grey solid. MS (ESI) m/z=180.1 [M+H]⁺.

Step 10. Synthesis of tert-butyl (3-(trans-3-(4-(3-chloropyridin-2-yl)-1H-pyrazol-1-yl)cyclobutyl)propyl)carbamate

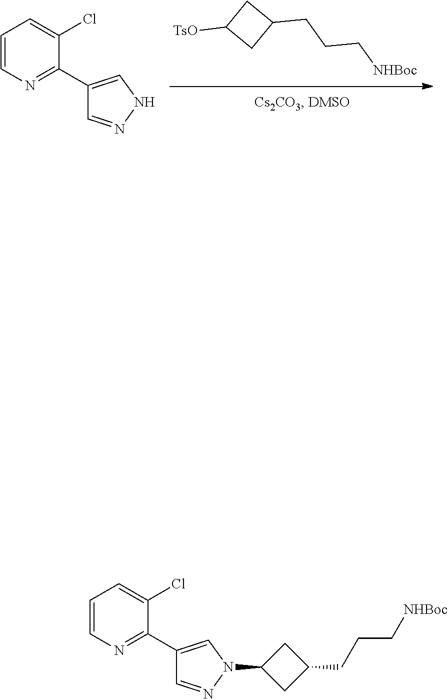

A mixture of 3-chloro-2-(1H-pyrazol-4-yl)pyridine (62 mg, 278.39 μmol), 3-(3-((tert-butoxycarbonyl)amino)propyl)cyclobutyl 4-methylbenzenesulfonate (150 mg, 367.67 μmol) and Cs₂CO₃ (181.40 mg, 556.77 μmol) in DMSO (2 mL) was heated at 90° C. under N₂ for 1 h. After cooling down to rt, the mixture was purified by reverse phase chromatography to provide the title compound (65 mg, yield: 45%) as a light-brown oil. MS (ESI) m/z=391.4 [M+H]⁺.

Step 11. Synthesis of 5-((3-(trans-3-(4-(3-chloropyridin-2-yl)-1H-pyrazol-1-yl)cyclobutyl)propyl)amino)-2-(2,6-dioxopiperidin-3-yl)isoindoline-1,3-dione

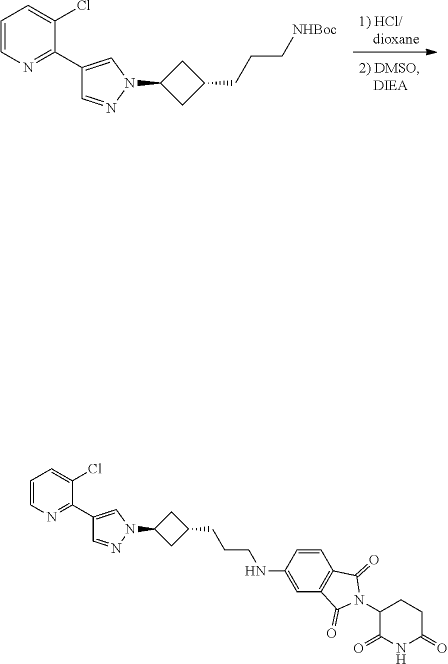

To a solution of tert-butyl (3-(trans-3-(4-(3-chloropyridin-2-yl)-1H-pyrazol-1-yl)cyclobutyl)propyl)carbamate (60 mg, 116.65 μmol) in DCM (5 mL) was added HCl solution (4 M in dioxane, 2 mL) at rt. After stirring at rt for 1 h, the mixture was concentrated under reduced pressure. To the resulting residue were added DIEA (80 mg, 620.16 μmol), 2-(2,6-dioxo-3-piperidyl)-5-fluoro-isoindoline-1,3-dione (65 mg, 235.32 μmol) and DMSO (3 mL). The mixture was heated at 150° C. for 2 h under N₂. After cooling down to rt, the mixture was purified by reverse phase chromatography to provide the title compound (10 mg, yield: 16%) as a yellow solid. MS (ESI) m/z=547.7 [M+H]⁺.

### Example 566. 3-(3-Cyclopropyl-4-(quinoxalin-2-yl)-1H-pyrazol-1-yl)-N-((2-(2,6-dioxopiperidin-3-yl)-1,3-dioxoisoindolin-5-yl)methyl)propanamide (GS-312)

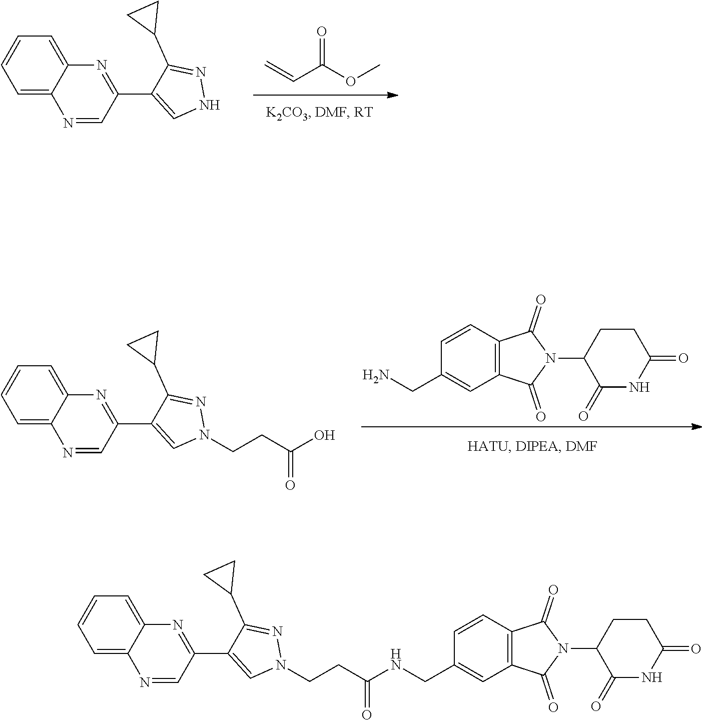

Step 1. Synthesis of 3-(3-cyclopropyl-4-(quinoxalin-2-yl)-1H-pyrazol-1-yl)propanoic acid

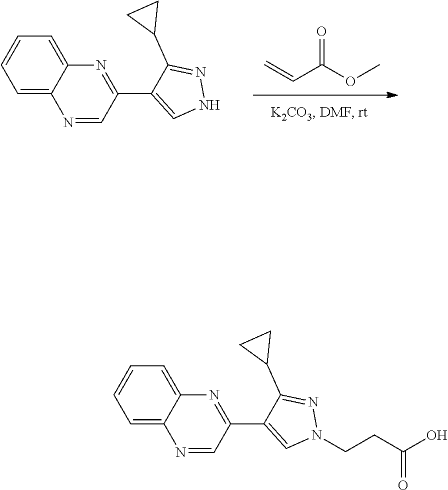

To a solution of 2-(3-cyclopropyl-1H-pyrazol-4-yl)quinoxaline (200 mg, 846.49 μmol) in DMF (5 mL) was added NaH (40.63 mg, 1.69 mmol) at rt. After stirring at rt for 30 min, methyl acrylate (72.87 mg, 846.49 μmol) was added. The obtained mixture was stirred at rit for 16 h, before it was quenched with water. The resulting mixture was purified by reverse phase chromatography to provide the title compound (30 mg, yield: 11%) as a yellow oil. MS (ESI) m/z=309.3 [M+H]⁺.

Step 2. Synthesis of 3-(3-cyclopropyl-4-(quinoxalin-2-yl)-1H-pyrazol-1-yl)-N-((2-(2,6-dioxopiperidin-3-yl)-1,3-dioxoisoindolin-5-yl)methyl)propanamide

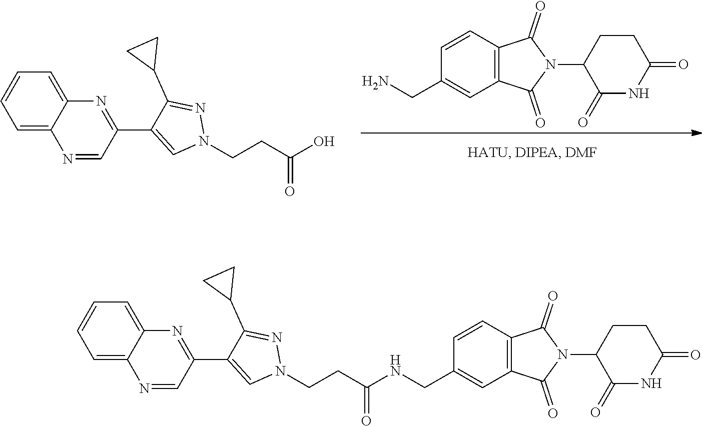

To a solution of 3-(3-cyclopropyl-4-(quinoxalin-2-yl)-1H-pyrazol-1-yl)propanoic acid (5 mg, 16.22 μmol) in DMF (1 mL) were added 5-(aminomethyl)-2-(2,6-dioxopiperidin-3-yl)isoindoline-1,3-dione (4.66 mg, 16.22 μmol), HATU (9.24 mg, 24.32 μmol) and DIPEA (6.29 mg, 48.65 μmol). After the mixture was stirred at rt for 30 min, it was purified by reverse phase chromatography to provide the title compound (5 mg, yield: 53%) as a yellow solid. MS (ESI) m/z=578.5 [M+H]⁺.

### Example 567. 5-(((3-(3-Cyclopropyl-4-(quinoxalin-2-yl)-1H-pyrazol-1-yl)propyl)amino)methyl)-2-(2,6-dioxopiperidin-3-yl)isoindoline-1,3-dione (GS-313)

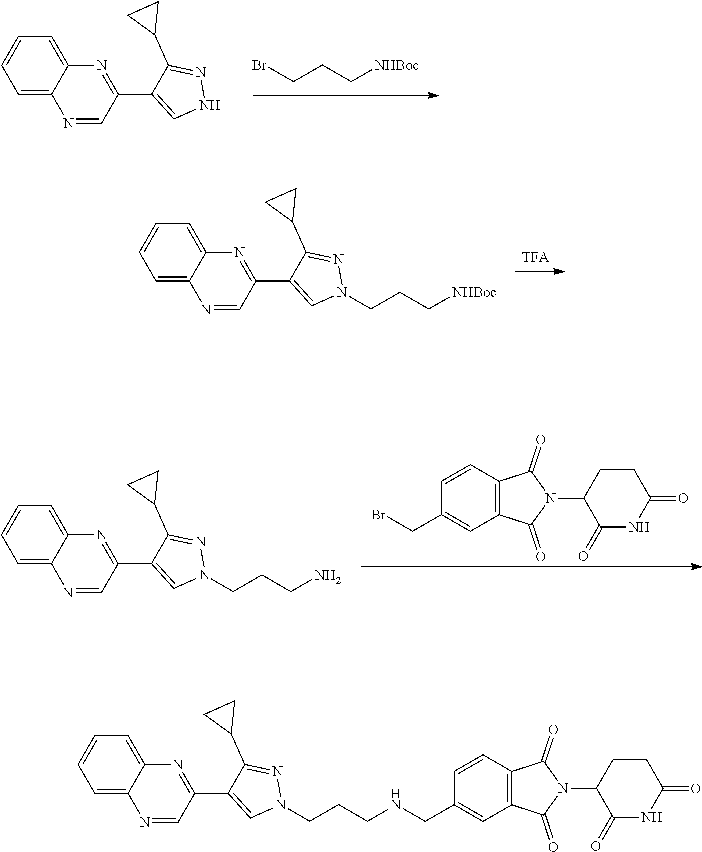 [SMILES: c1ccc2nc(-c3c[nH]nc3C3CC3)cnc2c1 ; CC(C)(C)OC(=O)NCCCBr ; CC(C)(C)OC(=O)NCCCn1cc(-c2cnc3ccccc3n2)c(C2CC2)n1 ; NCCCn1cc(-c2cnc3ccccc3n2)c(C2CC2)n1 ; O=C1CCC(N2C(=O)c3ccc(CBr)cc3C2=O)C(=O)N1 ; O=C1CCC(N2C(=O)c3ccc(CNCCCn4cc(-c5cnc6ccccc6n5)c(C5CC5)n4)cc3C2=O)C(=O)N1]

Step 1 to 2. Synthesis of 3-(3-cyclopropyl-4-(quinoxalin-2-yl)-1H-pyrazol-1-yl)propan-1-amine

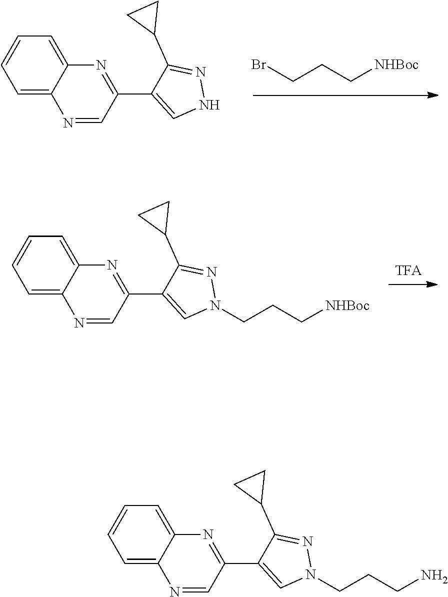 [SMILES: c1ccc2nc(-c3c[nH]nc3C3CC3)cnc2c1 ; CC(C)(C)OC(=O)NCCCBr ; CC(C)(C)OC(=O)NCCCn1cc(-c2cnc3ccccc3n2)c(C2CC2)n1 ; NCCCn1cc(-c2cnc3ccccc3n2)c(C2CC2)n1]

The title compound was synthesized following the standard procedures for preparing GS-326 (10 mg) as a yellow oil. MS (ESI) m/z=294.4 [M+H]⁺.

Step 3. Synthesis of 5-(((3-(3-cyclopropyl-4-(quinoxalin-2-yl)-1H-pyrazol-1-yl)propyl)amino)methyl)-2-(2,6-dioxopiperidin-3-yl)isoindoline-1,3-dione

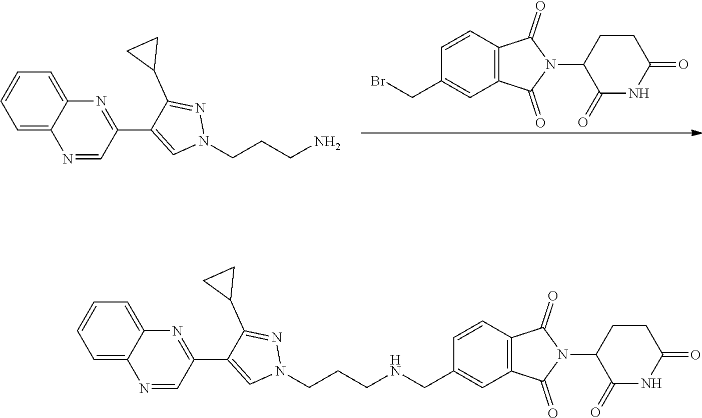 [SMILES: NCCCn1cc(-c2cnc3ccccc3n2)c(C2CC2)n1 ; O=C1CCC(N2C(=O)c3ccc(CBr)cc3C2=O)C(=O)N1 ; O=C1CCC(N2C(=O)c3ccc(CNCCCn4cc(-c5cnc6ccccc6n5)c(C5CC5)n4)cc3C2=O)C(=O)N1]

To a solution of 3-(3-cyclopropyl-4-(quinoxalin-2-yl)-1H-pyrazol-1-yl)propan-1-amine (5 mg, 17.04 μmol) in DMSO (1 mL) were added 5-(bromomethyl)-2-(2,6-dioxo-3-piperidyl)isoindoline-1,3-dione (5.98 mg, 17.04 μmol) and DIPEA (4.41 mg, 34.09 μmol). The mixture was stirred at 150° C. for 1 h. After cooling down to rt, the mixture was purified by reverse phase chromatography to provide the title compound (2 mg, yield: 21%) as a white solid. MS (ESI) m/z=564.4 [M+H]⁺.

### Example 568. 5-(((trans-3-(3-Cyclopropyl-4-(6-(4-methylpiperazin-1-yl)quinoxalin-2-yl)-1H-pyrazol-1-yl)cyclobutyl)methyl)amino)-2-(2,6-dioxopiperidin-3-yl)isoindoline-1,3-dione (GS-314)

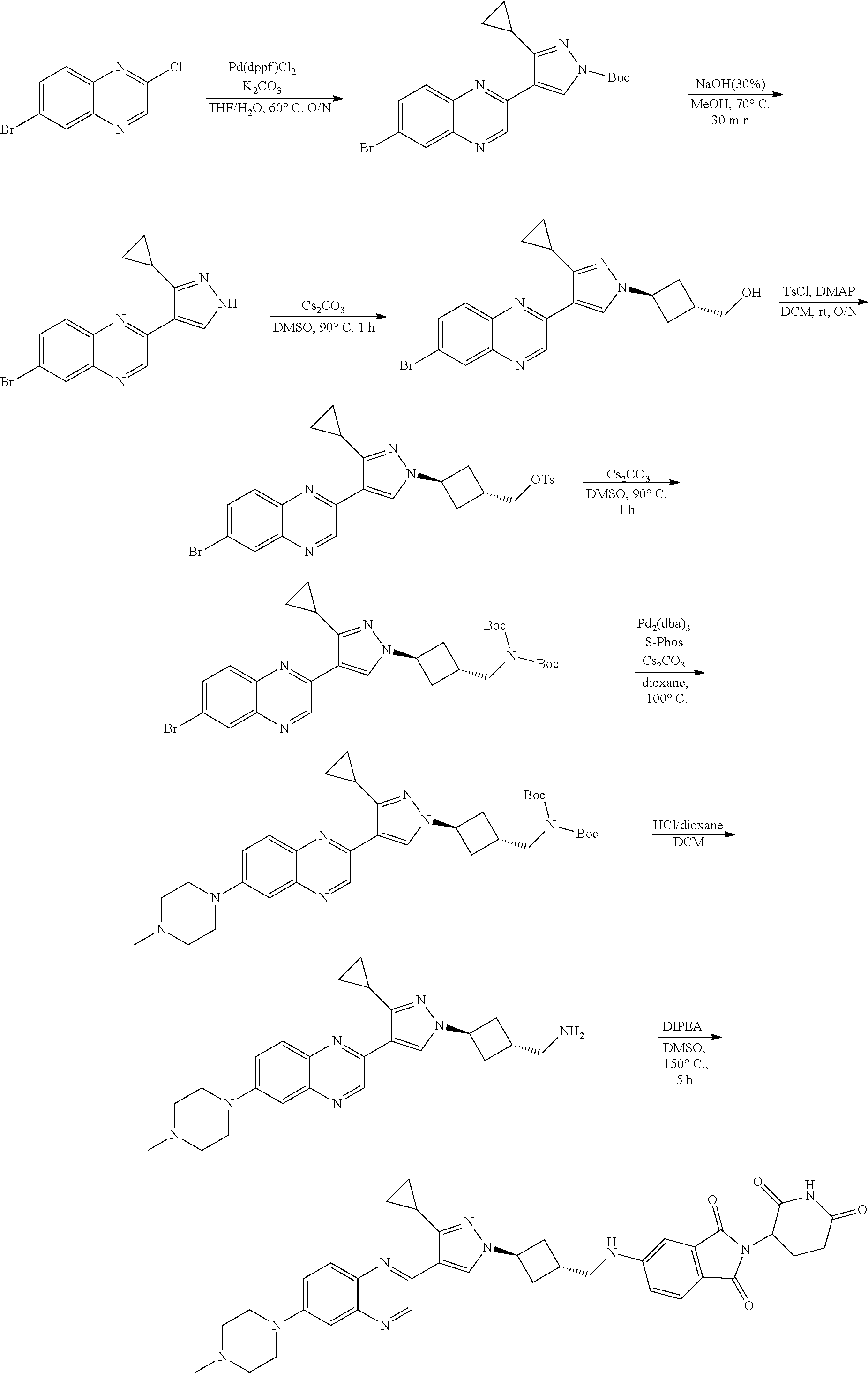

Step 1. Synthesis of tert-butyl 4-(6-bromoquinoxalin-2-yl)-3-cyclopropyl-1H-pyrazole-1-carboxylate

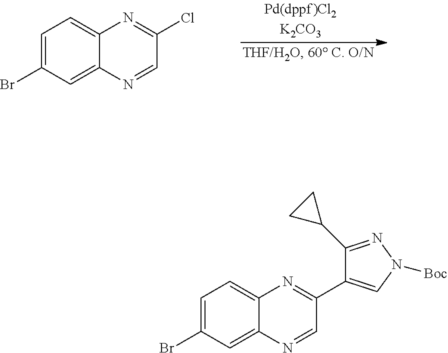 [SMILES: Clc1cnc2cc(Br)ccc2n1 ; CC(C)(C)OC(=O)n1cc(-c2cnc3cc(Br)ccc3n2)c(C2CC2)n1]

To a mixture of 6-bromo-2-chloro-quinoxaline (2.0 g, 8.2 mmol) and tert-butyl 3-cyclopropyl-4-(4,4,5,5-tetramethyl-1,3,2-dioxaborolan-2-yl)pyrazole-1-carboxylate (2.7 g, 8.2 mmol) in THF (20 mL) and water (4 mL) were added Pd(dppf)Cl₂ (764 mg, 821 μmol) and K₂CO₃ (2.27 g, 16.4 mmol). The reaction mixture was stirred at 60° C. overnight. After cooling down to rt, the solution was diluted with water (20 mL) and extracted with ethyl acetate (20 mL×3). The combined organic phase was washed with brine, dried over anhydrous sodium sulfate, filtered, and concentrated under reduced pressure to provide the crude compound (2.0 g, yield: 59%) which was used directly in the next step. MS (ESI) m/z=417.2 [M+H]⁺.

Step 2. Synthesis of 6-bromo-2-(3-cyclopropyl-1H-pyrazol-4-yl)quinoxaline

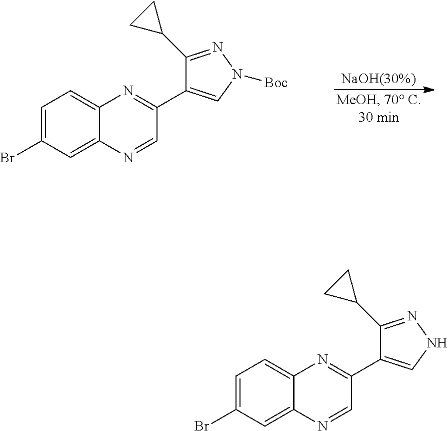 [SMILES: CC(C)(C)OC(=O)n1cc(-c2cnc3cc(Br)ccc3n2)c(C2CC2)n1 ; Brc1ccc2nc(-c3c[nH]nc3C3CC3)cnc2c1]

To a solution of tert-butyl 4-(6-bromoquinoxalin-2-yl)-3-cyclopropyl-1-pyrazole-1-carboxylate (2.0 g, 4.8 mmol) in methanol (20 mL) was added aq. NaOH (30%, 1.3 mL) solution. The reaction mixture was stirred at 70° C. for 30 min. The mixture was diluted with water and concentrated most of MeOH under reduced pressure. The resulting residue was extracted with MTBE. The combined organic layer was washed with brine, dried over Na₂SO₄, filtered, and concentrated to provide the crude compound (1 g, yield: 66%) as a black solid. MS (ESI) m/z=317.1 [M+H]⁺.

Step 3. Synthesis of (trans-3-(4-(6-bromoquinoxalin-2-yl)-3-cyclopropyl-1H-pyrazol-1-yl)cyclobutyl)methanol

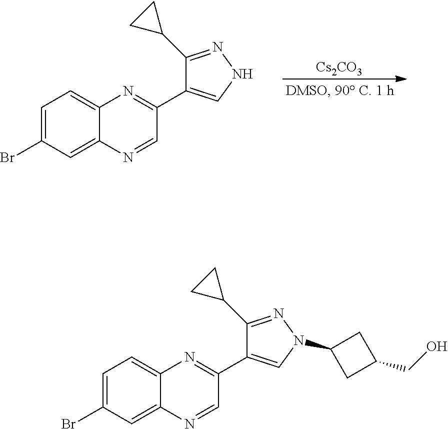

To a mixture of 6-bromo-2-(3-cyclopropyl-1H-pyrazol-4-yl)quinoxaline (1.0 g, 3.1 mmol) and 3-(hydroxymethyl)cyclobutyl 4-methylbenzenesulfonate (1.0 g, 4.1 mmol) in DMSO (10 mL) was added Cs₂CO₃ (2.0 g, 6.3 mmol). The mixture was stirred at 90° C. for 1 h. After cooling down to rt, the reaction was quenched with water (50 mL) and extrated with EtOAc (30 mL×3). The combined organic layer was washed with water and brine, dried over Na₂SO₄, filtered, and concentrated to provide the crude compound (400 mg, yield: 32%) as a brown solid. MS (ESI) m/z=401.2 [M+H]⁺.

Step 4. Synthesis of (trans-3-(4-(6-bromoquinoxalin-2-yl)-3-cyclopropyl-1H-pyrazol-1-yl)cyclobutyl)methyl 4-methylbenzenesulfonate

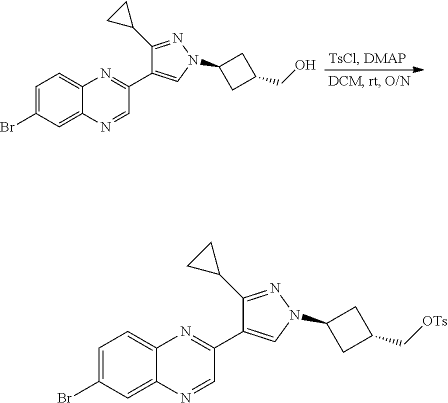 [SMILES: OC[C@H]1C[C@H](n2cc(-c3cnc4cc(Br)ccc4n3)c(C3CC3)n2)C1 ; Cc1ccc(S(=O)(=O)OC[C@H]2C[C@H](n3cc(-c4cnc5cc(Br)ccc5n4)c(C4CC4)n3)C2)cc1]

To a mixture of (trans-3-(4-(6-bromoquinoxalin-2-yl)-3-cyclopropyl-1H-pyrazol-1-yl)cyclobutyl)methanol (400 mg, 1.0 mmol) and TsCl (70 mg, 370 μmol) in DCM (5 mL) were added DMAP (12 mg, 100 μmol) and DIPEA (388 mg, 3.0 mmol). After stirring at rt overnight, the mixture was quenched with water (5 mL) and extracted with DCM (10 mL×3). The combined organic layer was washed with brine, dried over Na₂SO₄, filtered, and concentrated under reduced pressure. The resulting residue was purified by reverse phase chromatography to provide the title compound (200 mg, yield: 36%) as a yellow solid. MS (ESI) m/z=555.1 [M+H]⁺.

Step 5. Synthesis of tert-butyl N-[[trans-3-[4-(6-bromoquinoxalin-2-yl)-3-cyclopropyl-pyrazol-1-yl]cyclobutyl]methyl]-N-tert-butoxycarbonyl-carbamate

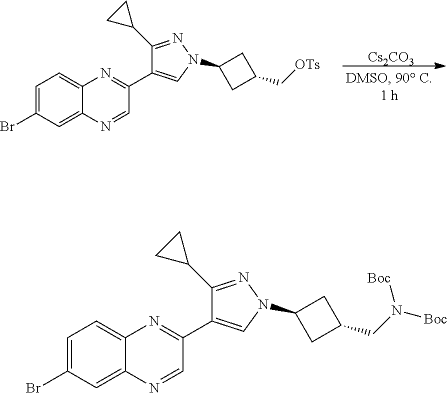

To a mixture of (trans-3-(4-(6-bromoquinoxalin-2-yl)-3-cyclopropyl-1H-pyrazol-1-yl)cyclobutyl)methyl 4-methylbenzenesulfonate (200 mg, 361 μmol) and tert-butyl N-tert-butoxycarbonylcarbamate (94 mg, 433 μmol) in DMSO (4 mL) was added Cs₂CO₃ (235 mg, 723 μmol). The reaction mixture was stirred at 90° C. for 1 h. After cooling down to rt, the resulting residue was purified by reverse phase chromatography to provide the title compound (200 mg, yield: 92%) as a brown solid. MS (ESI) m/z=600.3 [M+H]⁺.

Step 6. Synthesis of tert-butyl N-tert-butoxycarbonyl-N-[[3-[3-cyclopropyl-4-[6-(4-methylpiperazin-1-yl)quinoxalin-2-yl]pyrazol-1-yl]cyclobutyl]methyl]carbamate

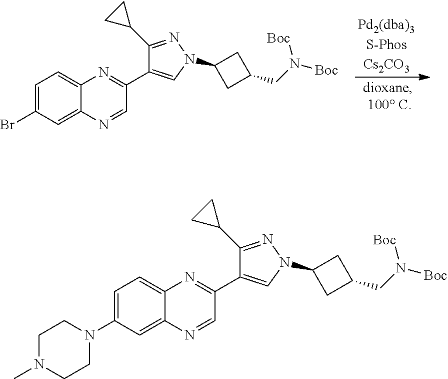 [SMILES: CC(C)(C)OC(=O)N(C[C@H]1C[C@H](n2cc(-c3cnc4cc(Br)ccc4n3)c(C3CC3)n2)C1)C(=O)OC(C)(C)C ; CN1CCN(c2ccc3nc(-c4cn([C@H]5C[C@H](CN(C(=O)OC(C)(C)C)C(=O)OC(C)(C)C)C5)nc4C4CC4)cnc3c2)CC1]

To a mixture of tert-butyl N-[[trans-3-[4-(6-bromoquinoxalin-2-yl)-3-cyclopropyl-pyrazol-1-yl]cyclobutyl]methyl]-N-tert-butoxycarbonyl-carbamate (100 mg, 167 μmol) and 1-methylpiperazine (33 mg, 334 μmol) in dioxane (3 mL) were added s-Phos (14 mg, 33 μmol), Pd₂(dba)₃ (15 mg, 17 μmol) and Cs₂CO₃ (109 mg, 334 μmol). The reaction mixture was stirred at 100° C. for 1 h. After cooling down to rt, the solution was quenched with water (5 mL) and extracted with EtOAc (10 mL×3). The combined organic phase was washed with brine, dried over Na₂SO₄, filtered, and concentrated under reduced pressure. The resulting residue was purified by silica gel chromatography (petroleum ether/EtOAc=7:3 to 0:1) to provide the title compound (80 mg, yield: 78%) as a yellow solid. MS (ESI) m/z=618.6 [M+H]⁺.

Step 7. Synthesis of (trans-3-(3-cyclopropyl-4-(6-(4-methylpiperazin-1-yl)quinoxalin-2-yl)-1H-pyrazol-1-yl)cyclobutyl)methanamine

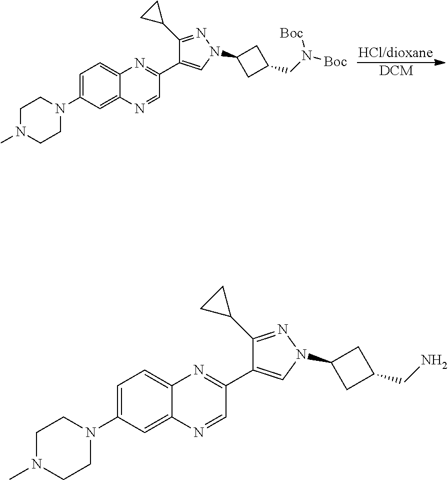 [SMILES: CN1CCN(c2ccc3nc(-c4cn([C@H]5C[C@H](CN(C(=O)OC(C)(C)C)C(=O)OC(C)(C)C)C5)nc4C4CC4)cnc3c2)CC1 ; CN1CCN(c2ccc3nc(-c4cn([C@H]5C[C@H](CN)C5)nc4C4CC4)cnc3c2)CC1]

To a solution of tert-butyl N-tert-butoxycarbonyl-N-[[3-[3-cyclopropyl-4-[6-(4-methylpiperazin-1-yl)quinoxalin-2-yl]pyrazol-1-yl]cyclobutyl]methyl]carbamate (80 mg, 129 μmol) in DCM (1 mL) was added HCl solution (4 M in dioxane, 1 mL). The reaction was stirred at rt for 10 min. The mixture was concentrated to provide the crude compound (50 mg, yield: 92%) as a red solid. MS (ESI) m/z=418.5 [M+H]⁺.

Step 8. Synthesis of 5-(((trans-3-(3-cyclopropyl-4-(6-(4-methylpiperazin-1-yl)quinoxalin-2-yl)-1H-pyrazol-1-yl)cyclobutyl)methyl)amino)-2-(2,6-dioxopiperidin-3-yl)isoindoline-1,3-dione

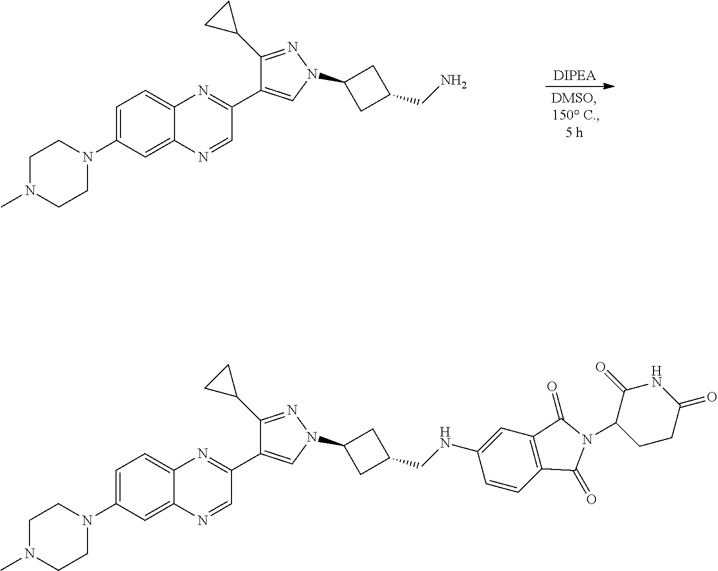 [SMILES: CN1CCN(c2ccc3nc(-c4cn([C@H]5C[C@H](CN)C5)nc4C4CC4)cnc3c2)CC1 ; CN1CCN(c2ccc3nc(-c4cn([C@H]5C[C@H](CNc6ccc7c(c6)C(=O)N(C6CCC(=O)NC6=O)C7=O)C5)nc4C4CC4)cnc3c2)CC1]

To a mixture of (trans-3-(3-cyclopropyl-4-(6-(4-methylpiperazin-1-yl)quinoxalin-2-yl)-1H-pyrazol-1-yl)cyclobutyl)methanamine (50 mg, 120 μmol) and 2-(2,6-dioxo-3-piperidyl)-5-fluoro-isoindoline-1,3-dione (33 mg, 120 μmol) in DMSO (2 mL) was added DIPEA (46 mg, 359 μmol). The reaction mixture was stirred at 150° C. for 5 h. After cooling down to rt, the mixture was purified by reverse phase chromatography to provide the title compound (6.3 mg, yield: 8%) as a yellow solid. MS (ESI) m/z=675.0 [M+H]⁺.

### Example 569. 2-(2,6-Dioxopiperidin-3-yl)-5-(((trans-3-(3-(6-methylpyridin-2-yl)-1H-pyrazol-1-yl)cyclobutyl)methyl)amino)isoindoline-1,3-dione (GS-065)

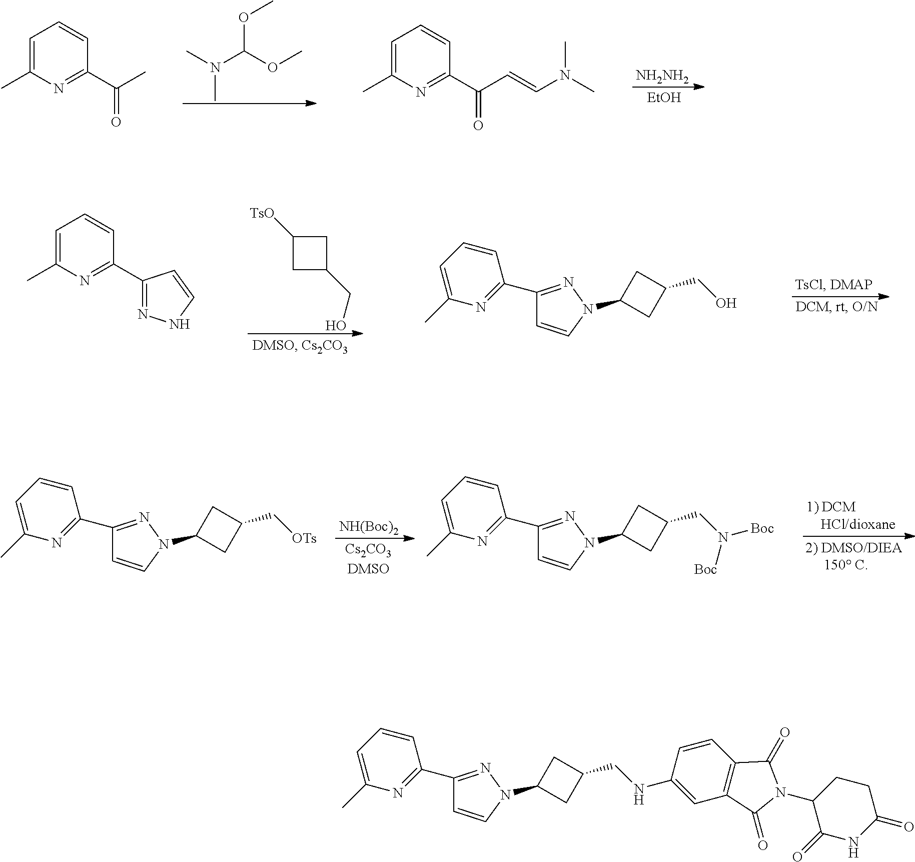

Step 1. Synthesis of 3-(dimethylamino)-1-(6-methylpyridin-2-yl)prop-2-en-1-one

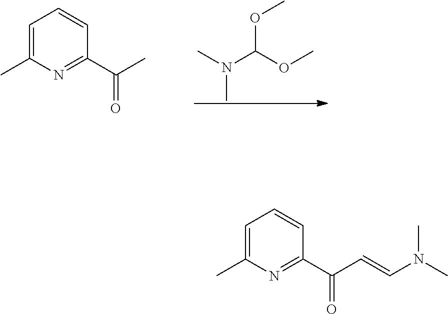

A mixture of 1-(6-methyl-2-pyridyl)ethanone (5 g, 36.99 mmol) and 1,1-dimethoxy-N,N-dimethyl-methanamine (7 g, 58.74 mmol) was heated at reflux overnight under N₂. The mixture was cooled and filtered to provide the title compound (5.0 g, yield: 71%) as a light-yellow solid. MS (ESI) m/z=192.2 [M+H]⁺.

Step 2. Synthesis of 2-methyl-6-(1H-pyrazol-3-yl)pyridine

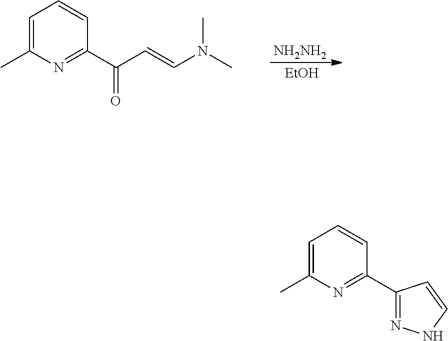

To a solution of 3-(dimethylamino)-1-(6-methylpyridin-2-yl)prop-2-en-1-one (2.5 g, 13.14 mmol) in ethanol (30 mL) was added hydrated hydrazine (5 mL) at rt. The mixture was heated at reflux for 1 h. After cooling down to rt, the solution was concentrated under reduced pressure to provide the crude compound (1.8 g, yield: 86%) as a white solid. MS (ESI) m/z=160.1 [M+H]⁺. The remaining steps were performed according to the standard procedures for preparing GS-314 (31 mg) to provide the desired final product as a yellow solid. MS (ESI) m/z=499.8 [M+H]⁺.

### Example 570. 5-(((trans-3-(3-Cyclopropyl-4-(6-morpholinoquinoxalin-2-yl)-1H-pyrazol-1-yl)cyclobutyl)methyl)amino)-2-(2,6-dioxopiperidin-3-yl)isoindoline-1,3-dione (GS-066)

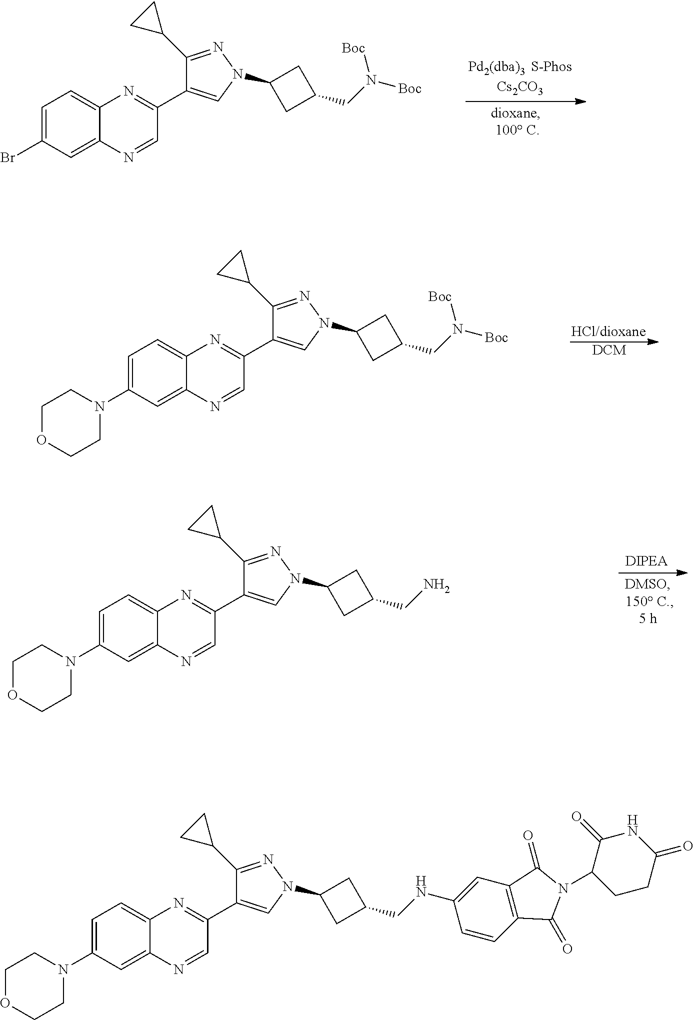 [SMILES: CC(C)(C)OC(=O)N(C[C@H]1C[C@H](n2cc(-c3cnc4cc(Br)ccc4n3)c(C3CC3)n2)C1)C(=O)OC(C)(C)C ; CC(C)(C)OC(=O)N(C[C@H]1C[C@H](n2cc(-c3cnc4cc(N5CCOCC5)ccc4n3)c(C3CC3)n2)C1)C(=O)OC(C)(C)C ; NC[C@H]1C[C@H](n2cc(-c3cnc4cc(N5CCOCC5)ccc4n3)c(C3CC3)n2)C1 ; O=C1CCC(N2C(=O)c3ccc(NC[C@H]4C[C@H](n5cc(-c6cnc7cc(N8CCOCC8)ccc7n6)c(C6CC6)n5)C4)cc3C2=O)C(=O)N1]

GS-066 was synthesized following the standard procedures for preparing GS-314 (0.47 mg) as a yellow solid. MS (ESI) m/z=661.8 [M+H]⁺.

### Example 571. 5-(((trans-3-(3-Cyclopropyl-4-(7-(morpholinomethyl)quinoxalin-2-yl)-1H-pyrazol-1-yl)cyclobutyl)methyl)amino)-2-(2,6-dioxopiperidin-3-yl)isoindoline-1,3-dione (GS-067)

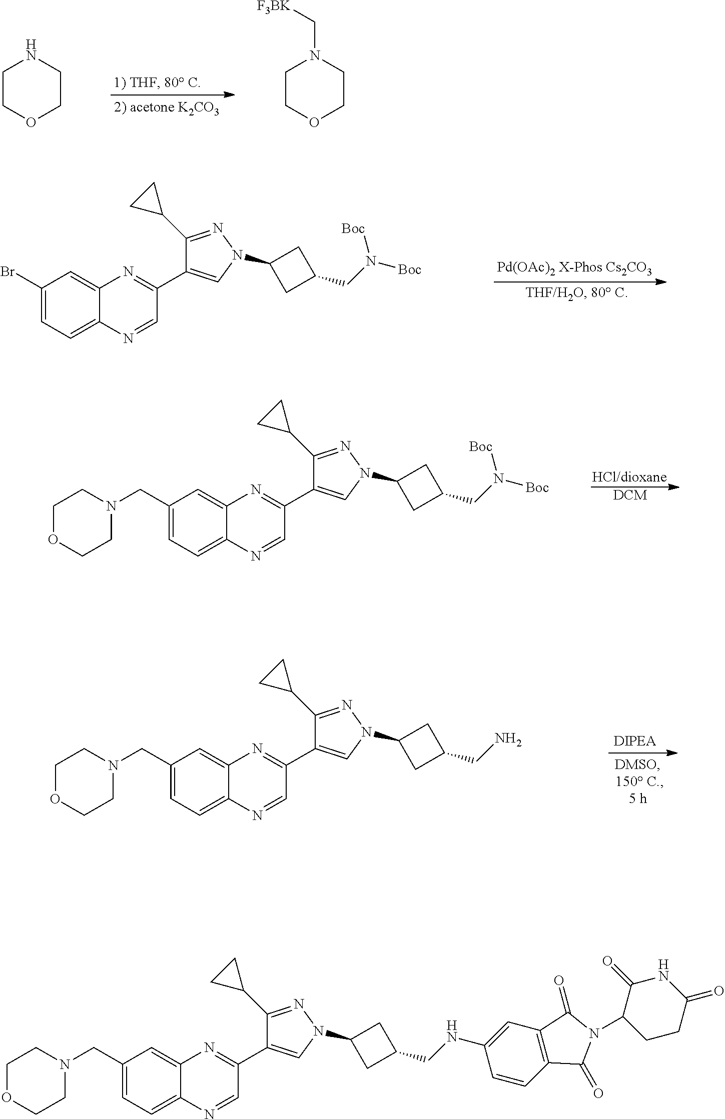

Step 1. Synthesis of potassium trifluoro(morpholinomethyl)borate

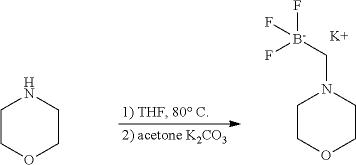 [SMILES: C1COCCN1 ; F[B-](F)(F)CN1CCOCC1 ; [K+]]

A solution of morpholine (500 mg, 5.7 mmol) and bromomethylpotassiumn trifluoroborane (2.3 g, 11.5 mmol) in THF (8 mL) was stirred at 80° C. overnight. The mixture was concentrated under reduced pressure. The residue was dissolved in acetone (50 mL) and K₂CO₃ (1.58 g, 11.5 mmol) was added. After stirring at rt overnight, the mixture was filtered and the filtrate was concentrated under reduced pressure. The resulting residue was washed with MTBE to provide the title compound (160 mg, yield: 14%) as a white solid.

Step 2. Synthesis of tert-butyl N-tert-butoxycarbonyl-N-[[trans-3-[3-cyclopropyl-4-[7-(morpholinomethyl)quinoxalin-2-yl]pyrazol-1-yl]cyclobutyl]methyl]carbamate

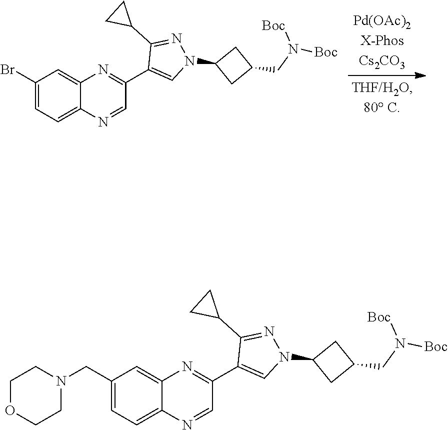 [SMILES: CC(C)(C)OC(=O)N(C[C@H]1C[C@H](n2cc(-c3cnc4ccc(Br)cc4n3)c(C3CC3)n2)C1)C(=O)OC(C)(C)C ; CC(C)(C)OC(=O)N(C[C@H]1C[C@H](n2cc(-c3cnc4ccc(CN5CCOCC5)cc4n3)c(C3CC3)n2)C1)C(=O)OC(C)(C)C]

To a mixture of tert-butyl N-[[trans-3-[4-(6-bromoquinoxalin-2-yl)-3-cyclopropyl-pyrazol-1-yl]cyclobutyl]methyl]-N-tert-butoxycarbonyl-carbamate (250 mg, 417 μmol) and potassium trifluoro(morpholinomethyl)borate (173 mg, 835 μmol) in THF (5 mL) and water (1 mL) were added Pd(OAc)₂ (9 mg, 41 μmol), X-Phos (40 mg, 83 μmol) and Cs₂CO₃ (272 mg, 835 μmol). The reaction mixture was stirred at 100° C. for 1 h. The solution was quenched with water (5 mL), and extracted with EtOAc (10 mL×3). The combined organic phase was washed with brine, dried over Na₂SO₄, filtered, and concentrated under reduced pressure. The resulting residue was purified by silica gel chromatography (petroleum ether/EtOAc=1:0 to 7:3) to provide the title compound (220 mg, yield: 85%) as a yellow solid. MS (ESI) m/z=619.5 [M+H]⁺.

The remaining steps were performed according to the standard procedures for preparing GS-314 (2.6 mg) to give the desired final product as a yellow solid. MS (ESI) m/z=675.8 [M+H]⁺.

### Example 572. 5-(((trans-3-(3-Cyclopropyl-4-(7-(morpholinomethyl)quinoxalin-2-yl)-1H-pyrazol-1-yl)cyclobutyl)methyl)amino)-2-(2,6-dioxopiperidin-3-yl)isoindoline-1,3-dione (GS-068)

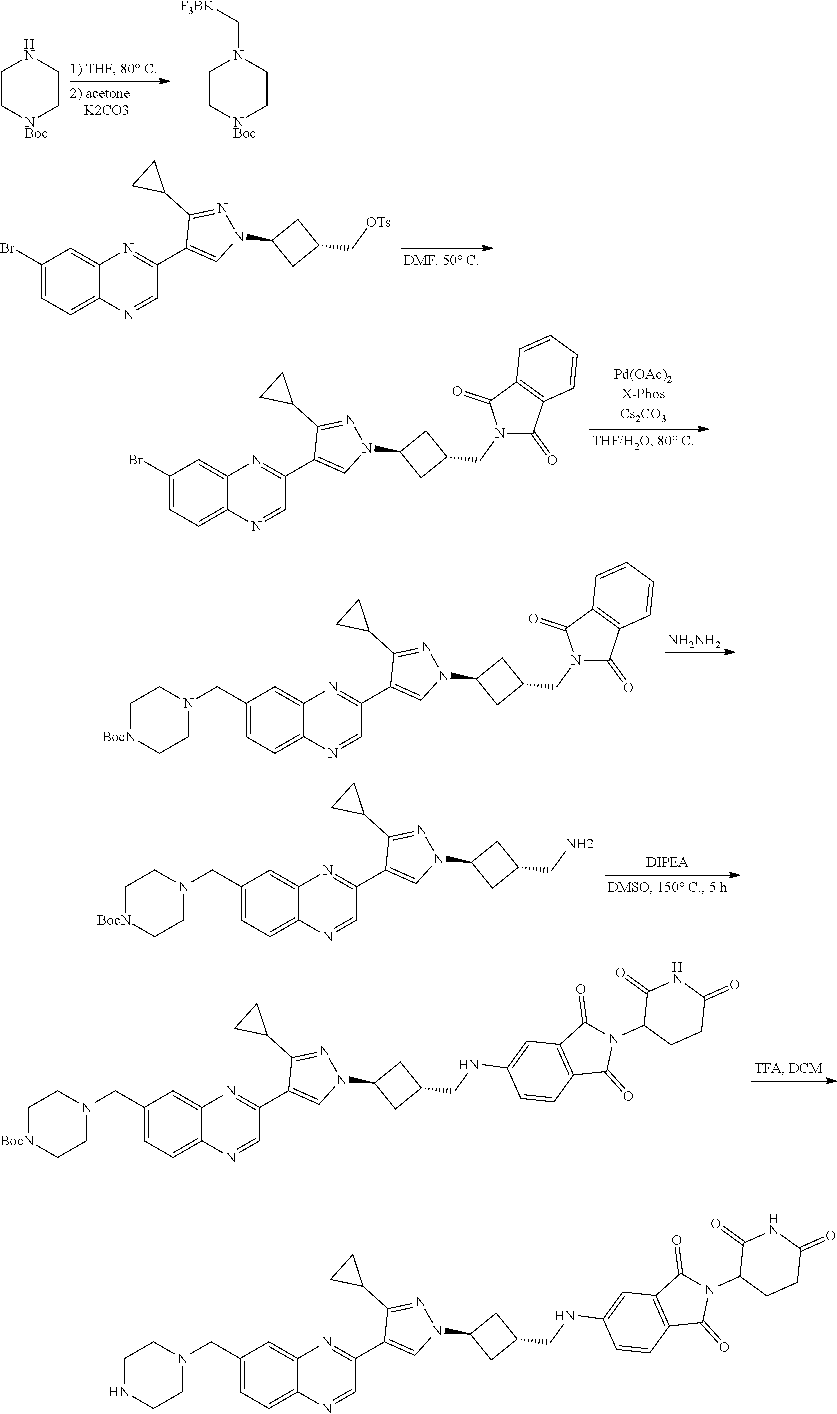

Step 1. Synthesis of potassium ((4-(tert-butoxycarbonyl)piperazin-1-yl)methyl)trimethylborate

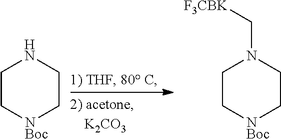

A solution of tert-butyl piperazine-1-carboxylate (500 mg, 2.7 mmol) and bromomethylpotassium trifluoroborane (1.0 g, 5.3 mmol) in THF (10 mL) was stirred at 80° C. overnight. The mixture was concentrated under reduced pressure. The residue was dissolved in acetone (50 mL) and K₂CO₃ (741 mg, 5.4 mmol) was added. After stirring at rt overnight, the solution was filtered and the filtrate was concentrated under reduced pressure. The resulting residue was washed with MTBE to provide the title compound (160 mg, yield: 20%) as a white solid.

Step 2. Synthesis of 2-((trans-3-(4-(7-bromoquinoxalin-2-yl)-3-cyclopropyl-1H-pyrazol-1-yl)cyclobutyl)methyl)isoindoline-1,3-dione

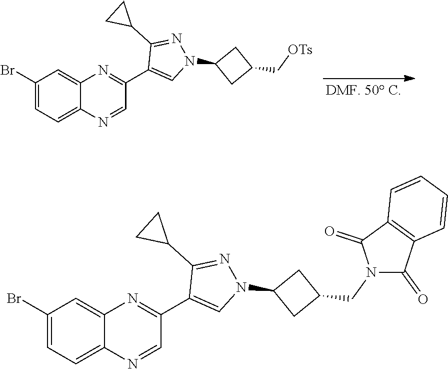 [SMILES: Cc1ccc(S(=O)(=O)OC[C@H]2C[C@H](n3cc(-c4cnc5ccc(Br)cc5n4)c(C4CC4)n3)C2)cc1 ; O=C1c2ccccc2C(=O)N1C[C@H]1C[C@H](n2cc(-c3cnc4ccc(Br)cc4n3)c(C3CC3)n2)C1]

A solution of (trans-3-(4-(7-bromoquinoxalin-2-yl)-3-cyclopropyl-1H-pyrazol-1-yl)cyclobutyl)methyl 4-methylbenzenesulfonate (400 mg, 1.0 mmol) and (1,3-dioxoisoindolin-2-yl)potassium (371 mg, 2.0 mmol) in DMSO (4 mL) was stirred at 50° C. for 1 h. The mixture was purified by reverse phase chromatography to provide the title compound (300 mg, yield: 57%) as a white solid. MS (ESI) m/z=530.1 [M+H]⁺.

Step 3. Synthesis of tert-butyl 4-((3-(3-cyclopropyl-1-(trans-3-((1,3-dioxoisoindolin-2-yl)methyl)cyclobutyl)-1H-pyrazol-4-yl)quinoxalin-6-yl)methyl)piperazine-1-carboxylate

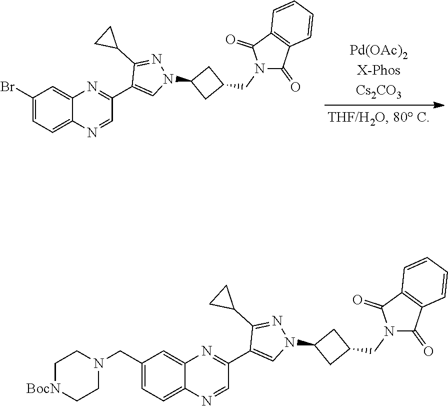 [SMILES: O=C1c2ccccc2C(=O)N1C[C@H]1C[C@H](n2cc(-c3cnc4ccc(Br)cc4n3)c(C3CC3)n2)C1 ; CC(C)(C)OC(=O)N1CCN(Cc2ccc3ncc(-c4cn([C@H]5C[C@H](CN6C(=O)c7ccccc7C6=O)C5)nc4C4CC4)nc3c2)CC1]

To a mixture of 2-((trans-3-(4-(7-bromoquinoxalin-2-yl)-3-cyclopropyl-1H-pyrazol-1-yl)cyclobutyl)methyl)isoindoline-1,3-dione (261 mg, 852 μmol) in water (1 mL) and THF (5 mL) were added Pd(OAc)₂ (13 mg, 56 μmol), X-Phos (54 mg, 113 μmol) and Cs₂CO₃ (370 mg, 1.1 mmol). The reaction mixture was stirred at 100° C. for 1 h. The solution was quenched with water (5 mL), and extracted with EtOAc (10 mL×3). The combined organic phase was washed with brine, dried over Na₂SO₄, filtered, and concentrated under reduced pressure. The resulting residue was purified by silica gel chromatography (petroleum ether/EtOAc=1:0 to 3:7) to provide the title compound (260 mg, yield: 71%) as a yellow solid. MS (ESI) m/z=648.5 [M+H]⁺.

Step 4. Synthesis of tert-butyl 4-((3-(1-(trans-3-(aminomethyl)cyclobutyl)-3-cyclopropyl-1H-pyrazol-4-yl)quinoxalin-6-yl)methyl)piperazine-1-carboxylate

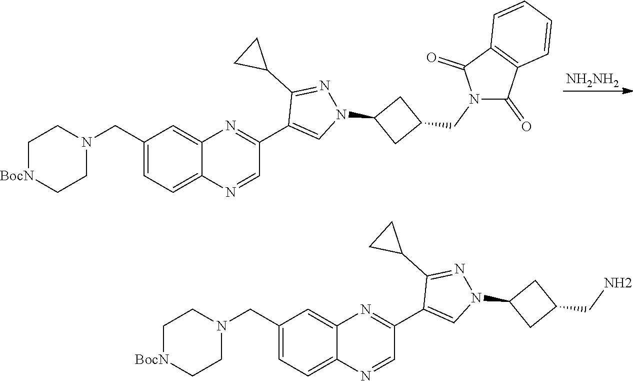

To a solution of tert-butyl 4-((3-(3-cyclopropyl-1-(trans-3-((1,3-dioxoisoindolin-2-yl)methyl)cyclobutyl)-1H-pyrazol-4-yl)quinoxalin-6-yl)methyl)piperazine-1-carboxylate (260 mg, 401 μmol) in EtOH (3 mL) was added NH₂NH₂ (0.5 mL). After stirring at rt for 1 h, the mixture was concentrated under reduced pressure to provide the title compound (200 mg, yield: 96%) which was used directly in the next step. MS (ESI) m/z=518.5 [M+H]⁺.

Step 5. Synthesis of tert-butyl 4-((3-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)quinoxalin-6-yl)methyl)piperazine-1-carboxylate

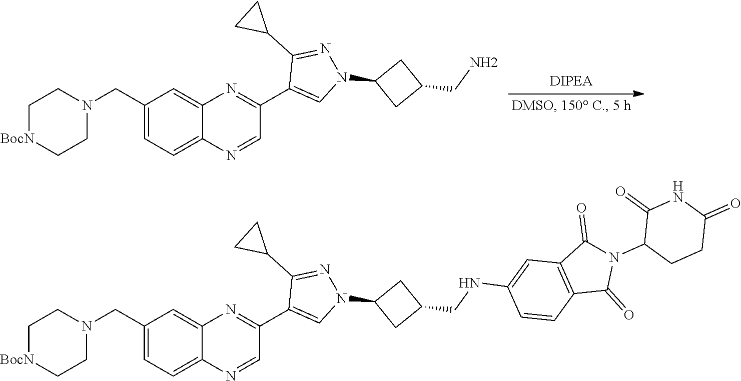 [SMILES: CC(C)(C)OC(=O)N1CCN(Cc2ccc3ncc(-c4cn([C@H]5C[C@H](CN)C5)nc4C4CC4)nc3c2)CC1 ; CC(C)(C)OC(=O)N1CCN(Cc2ccc3ncc(-c4cn([C@H]5C[C@H](CNc6ccc7c(c6)C(=O)N(C6CCC(=O)NC6=O)C7=O)C5)nc4C4CC4)nc3c2)CC1]

To a mixture of tert-butyl 4-((3-(1-(trans-3-(aminomethyl)cyclobutyl)-3-cyclopropyl-1H-pyrazol-4-yl)quinoxalin-6-yl)methyl)piperazine-1-carboxylate (200 mg, 386 μmol) and 2-(2,6-dioxo-3-piperidyl)-5-fluoro-isoindoline-1,3-dione (160 mg, 579 μmol) in DMSO (3 mL) was added DIPEA (50 mg, 386 μmol). The reaction mixture was stirred at 150° C. for 5 h. After cooling down to rt, the mixture was purified by reverse phase chromatography (0-70% MeCN in H₂O) to provide the title compound (10 mg, yield: 3.3%) as a yellow solid. MS (ESI) m/z=774.6 [M+H]⁺.

Step 6. Synthesis of 5-(((trans-3-(3-cyclopropyl-4-(7-(piperazin-1-ylmethyl)quinoxalin-2-yl)-1H-pyrazol-1-yl)cyclobutyl)methyl)amino)-2-(2,6-dioxopiperidin-3-yl)isoindoline-1,3-dione

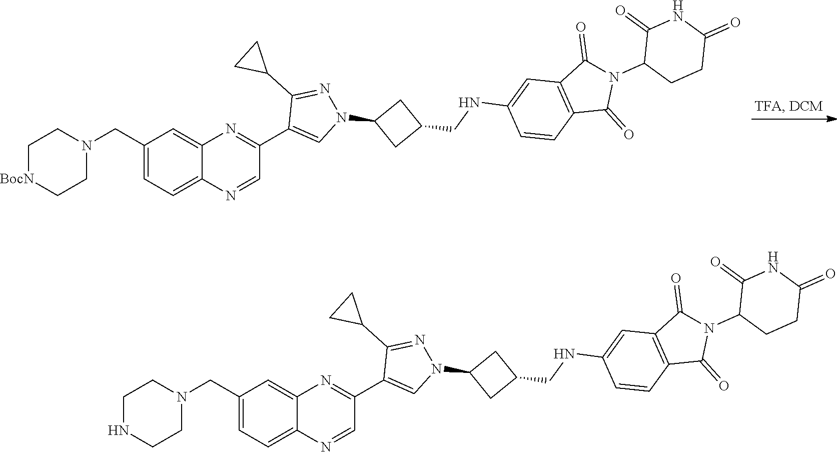 [SMILES: CC(C)(C)OC(=O)N1CCN(Cc2ccc3ncc(-c4cn([C@H]5C[C@H](CNc6ccc7c(c6)C(=O)N(C6CCC(=O)NC6=O)C7=O)C5)nc4C4CC4)nc3c2)CC1 ; O=C1CCC(N2C(=O)c3ccc(NC[C@H]4C[C@H](n5cc(-c6cnc7ccc(CN8CCNCC8)cc7n6)c(C6CC6)n5)C4)cc3C2=O)C(=O)N1]

To a solution of tert-butyl 4-((3-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)quinoxalin-6-yl)methyl)piperazine-1-carboxylate (10 mg, 13 μmol) in DCM (1 mL) was added TFA (0.5 mL). The reaction mixture was stirred at rt for 10 min. The mixture was concentrated under reduced pressure. The resulting residue was purified by reverse phase chromatography (0-70% MeCN in H₂O) to provide the title compound (1.8 mg, yield: 18%) as a yellow solid. MS (ESI) m/z=674.8 [M+H]⁺.

### Example 573. 5-(((trans-3-(3-Cyclopropyl-4-(6-ethylpyridin-2-yl)-1H-pyrazol-1-yl)cyclobutyl)methyl)amino)-2-(2,6-dioxopiperidin-3-yl)isoindoline-1,3-dione (GS-069)

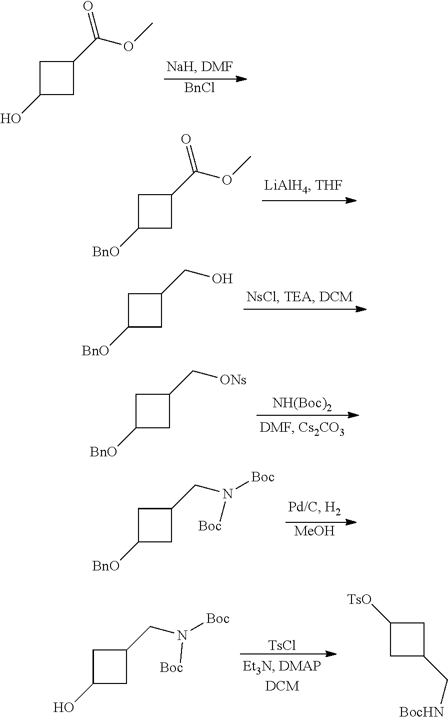

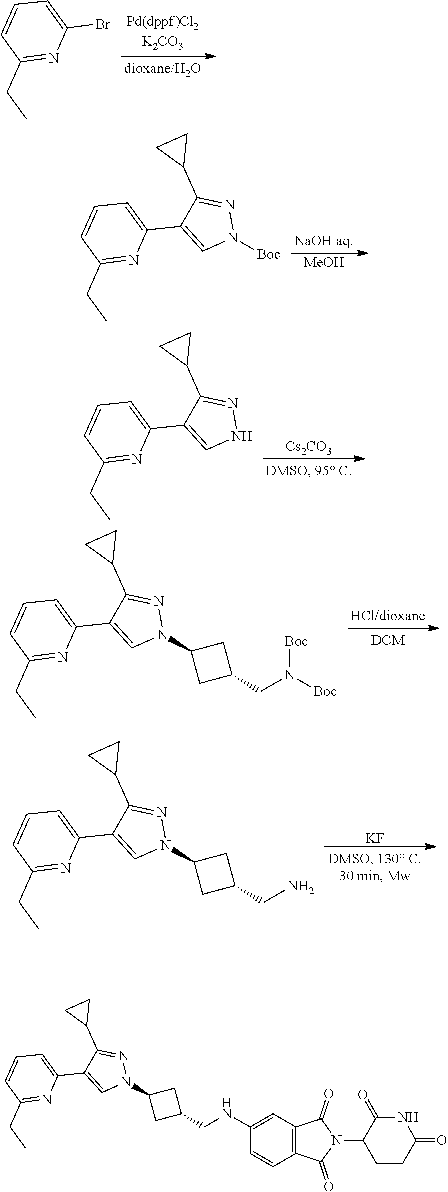

Step 1. Synthesis of methyl 3-(benzyloxy)cyclobutane-1-carboxylate

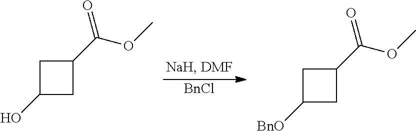

To a solution methyl 3-hydroxycyclobutanecarboxylate (10 g, 76.84 mmol) in DMF (100 mL) was added NaH (60% in mineral oil, 4.00 g, 99.89 mmol). The mixture was stirred at rt for 1 h. To the mixture was added bromomethylbenzene (14.46 g, 84.52 mmol) at 25° C. After stirring at rt for 18 h, the mixture was poured into cold aq. NH₄Cl solution (20%, 100 mL). The mixture was extracted with MTBE (500 mL). The organic layer was washed with water (200 mL×2), dried over anhydrous sodium sulfate, filtered, and concentrated under reduced pressure to provide the title compound (15 g, yield: 89%) as a yellow oil.

Step 2. Synthesis of (3-(benzyloxy)cyclobutyl)methanol

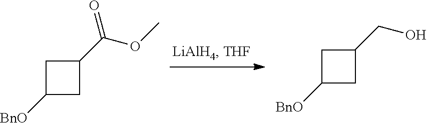 [SMILES: COC(=O)C1CC(OCc2ccccc2)C1 ; OCC1CC(OCc2ccccc2)C1]

To a suspension LiAlH₄ (5.18 g, 136.20 mmol) in THF (200 mL) was added methyl 3-(benzyloxy)cyclobutane-1-carboxylate (15 g, 68.10 mmol) at 25° C. After stirring at rt for 1 h, the reaction was quenched with water (10 mL) slowly. Silica gel (10 g) was added. The mixture was stirred for 10 min, before it was filtered. The filter cake was washed with THF (100 mL) and the filtrate was concentrated under reduced pressure. The residue was purified by silica gel chromatography (petroleum ether/EtOAc=10:1 to 3:1) to provide the title compound (4.5 g, yield: 34%) as a yellow oil. MS (ESI) m/z=193.2 [M+H]⁺.

Step 3. Synthesis of (3-(benzyloxy)cyclobutyl)methyl 4-nitrobenzenesulfonate

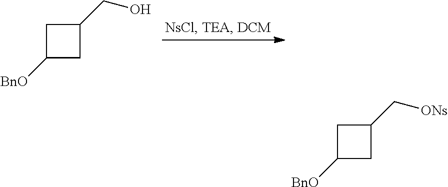

To a solution of (3-benzyloxycyclobutyl)methanol (47.8 g, 248.63 mmol) in DCM (500 mL) were added TEA (50.32 g, 497.26 mmol) and 4-nitrobenzenesulfonyl chloride (60.61 g, 273.49 mmol) at 25° C. After the mixture was stirred at rt for 18 h, it was washed with aq. Na₂CO₃ solution (5%, 500 mL). The organic layer was concentrated under reduced pressure to provide the title compound (75.3 g, yield: 80%) as a yellow solid.

Step 4. Synthesis of tert-butyl N-[(3-benzyloxycyclobutyl)methyl]-N-tert-butoxycarbonyl-carbamat

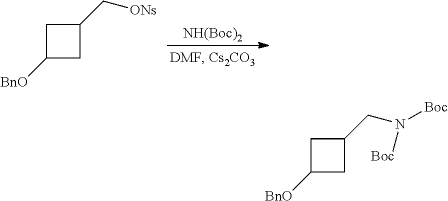

To a solution of (3-(benzyloxy)cyclobutyl)methyl 4-nitrobenzenesulfonate (70.00 g, 185.47 mmol) in DMF (200 mL) were added tert-butyl N-tert-butoxycarbonylcarbamate (24.18 g, 111.28 mmol) and Cs₂CO₃ (120.86 g, 370.95 mmol) at 25° C. The mixture was stirred at 100° C. for 2 h. After cooling down to rt, the mixture was diluted with EtOAc (500 mL) and washed with water (500 mL×4). The organic phase was concentrated under reduced pressure. The resulting residue was purified by silica gel chromatography (petroleum ether/EtOAc=100:1 to 10:1) to provide the title compound (32.5 g, yield: 45%) as a yellow oil. MS (ESI) m/z=192.23 [M−200+H]⁺.

Step 5. Synthesis of tert-butyl N-tert-butoxycarbonyl-N-[(3-hydroxycyclobutyl)methyl]carbamate

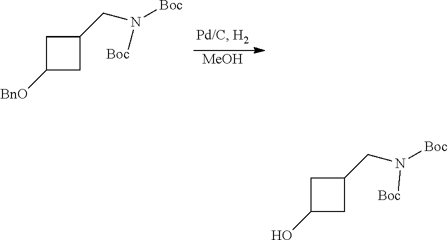 [SMILES: "MeOH" ; CC(C)(C)OC(=O)N(CC1CC(OCc2ccccc2)C1)C(=O)OC(C)(C)C ; CC(C)(C)OC(=O)N(CC1CC(O)C1)C(=O)OC(C)(C)C]

To a solution of tert-butyl N-[(3-benzyloxycyclobutyl)methyl]-N-tert-butoxycarbonyl-carbamate (1.00 g, 2.55 mmol) in methanol (20 mL) was added Pd/C (0.3 g) at 25° C. The mixture was purged with hydrogen and stirred at rt under hydrogen balloon for 48 h. The mixture was filtered through Celite. The filtrate was concentrated under reduced pressure to provide the crude compound (750 mg, yield: 97%) as a colorless oil.

Step 6. Synthesis of [3-[[bis(tert-butoxycarbonyl)amino]methyl]cyclobutyl] 4-methylbenzenesulfonate

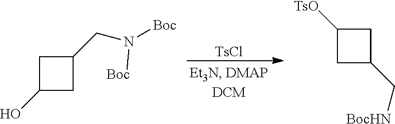

To a solution of tert-butyl N-tert-butoxycarbonyl-N-[(3-hydroxycyclobutyl)methyl]carbamate (6 g, 19.91 mmol), triethylamine (4.03 g, 39.82 mmol) and DMAP (243.22 mg, 1.99 mmol) in DCM (60 mL) was added p-toluenesulfonyl chloride (5.69 g, 29.86 mmol) at 0° C. After stirring at rt overnight, the mixture was washed with aq. NaHCO₃ solution (40 mL) and H₂O (40 mL). The organic phase was concentrated under reduced pressure. The resulting residue was purified by silica gel chromatography to provide the title compound (6 g, yield: 66%).

Step 7 to 10. Synthesis of (trans-3-(3-cyclopropyl-4-(6-ethylpyridin-2-yl)-1H-pyrazol-1-yl)cyclobutyl)methanamine

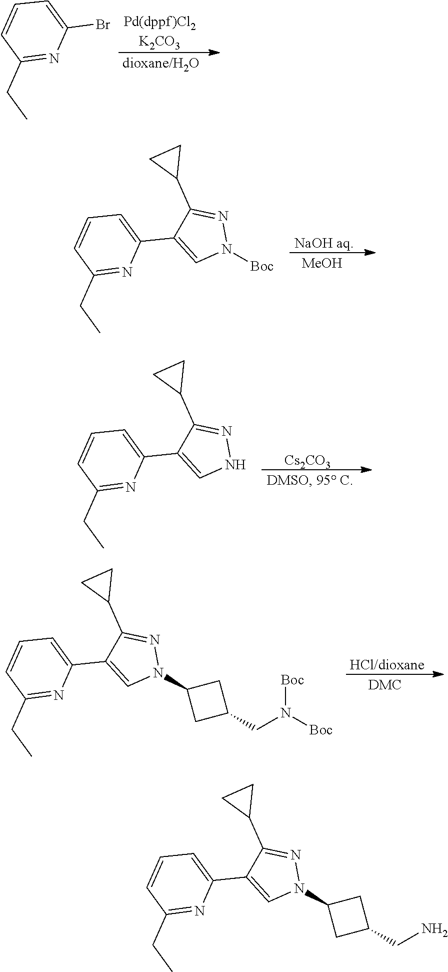

These steps were performed according to the standard procedures for preparing GS-314 (8 mg) to provide the title compound as a white solid. MS (ESI) m/z=297.3 [M+H]⁺.

Step 11. Synthesis of 5-(((trans-3-(3-cyclopropyl-4-(6-ethylpyridin-2-yl)-1H-pyrazol-1-yl)cyclobutyl)methyl)amino)-2-(2,6-dioxopiperidin-3-yl)isoindoline-1,3-dione

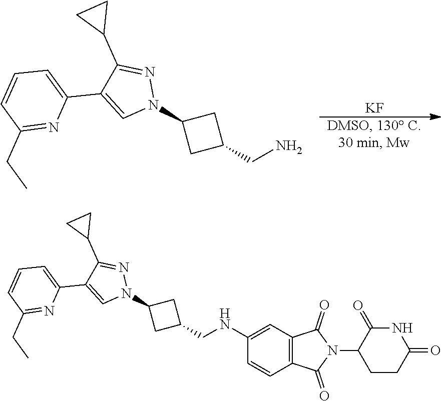 [SMILES: CCc1cccc(-c2cn([C@H]3C[C@H](CN)C3)nc2C2CC2)n1 ; "KF" ; CCc1cccc(-c2cn([C@H]3C[C@H](CNc4ccc5c(c4)C(=O)N(C4CCC(=O)NC4=O)C5=O)C3)nc2C2CC2)n1]

GS-069 was synthesized following the standard procedures for preparing GS-174 (2.0 mg, yield: 13%) as a yellow solid. MS (ESI) m/z=553.4 [M+H]⁺.

### Example 574. 5-((3-(trans-3-(4-(3-chloropyridin-2-yl)-3-cyclopropyl-1H-pyrazol-1-yl)cyclobutyl)propyl)amino)-2-(2,6-dioxopiperidin-3-yl)isoindoline-1,3-dione (GS-070)

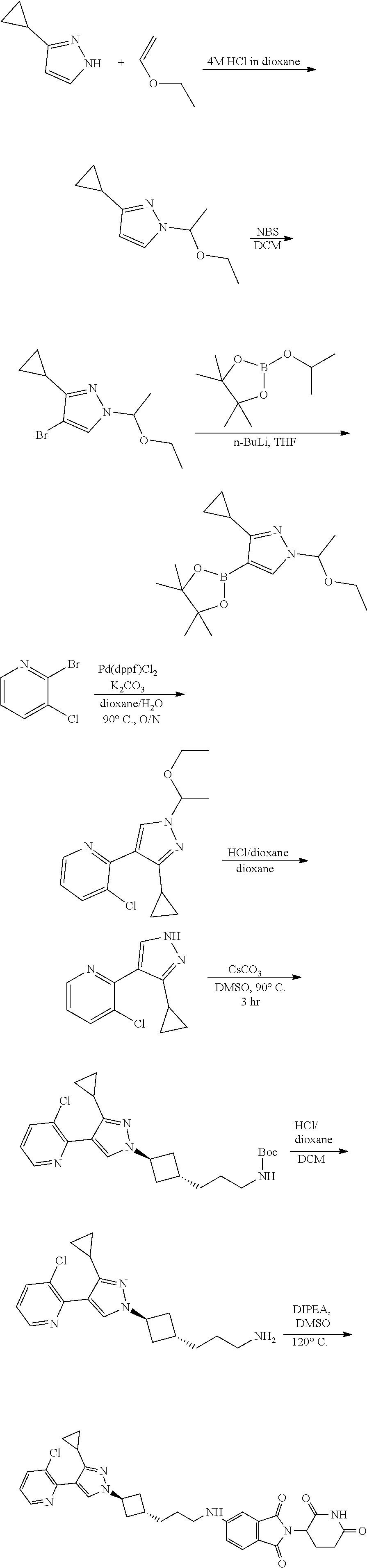

Step 1. Synthesis of 3-cyclopropyl-1-(1-ethoxyethyl)-1H-pyrazole

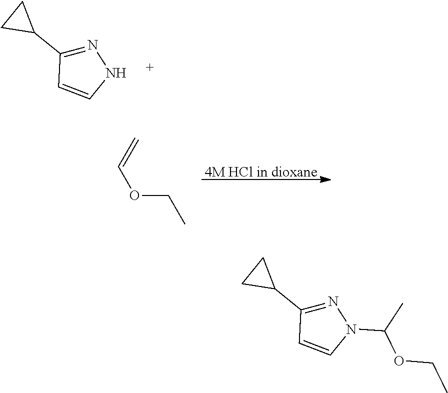

A solution of 3-cyclopropyl-1H-pyrazole (10 g, 92.47 mmol) and vinyloxyethane (8.67 g, 120.21 mmol), HCl solution (4 M in dioxane, 0.58 mL, 2.31 mmol) in toluene (120 mL) was stirred at 35-40° C. overnight. NaHCO₃ (7.77 g, 36.99 mmol) was added. After the mixture was stirred for 1 h, it was filtered. The filtrate was concentrated to provide the title compound (15.67 g, yield: 94%). MS (ESI) m/z=109.3 [M+H]⁺.

Step 2. Synthesis of 4-bromo-3-cyclopropyl-1-(1-ethoxyethyl)-1H-pyrazole

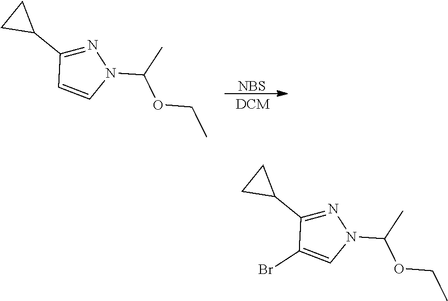

A solution of 3-cyclopropyl-1-(1-ethoxyethyl)-1H-pyrazole (14.7 g, 81.56 mmol) in DCM (140 mL) was added NBS (15.24 g, 85.63 mmol) in portions at 0-10° C. After the mixture was stirred at 0-10° C. for 1 h, aq. NaHSO₃ solution (100 mL) was added. The mixture was stirred for 30 min. The organic phase was separated, washed with aq. NaCl solution, dried, and concentrated to provide the title compound (20 g, yield: 95%). MS (ESI) m/z=189.1 [M+H]⁺.

Step 3. Synthesis of 3-cyclopropyl-1-(1-ethoxyethyl)-4-(4,4,5,5-tetramethyl-1,3,2-dioxaborolan-2-yl)-1H-pyrazole

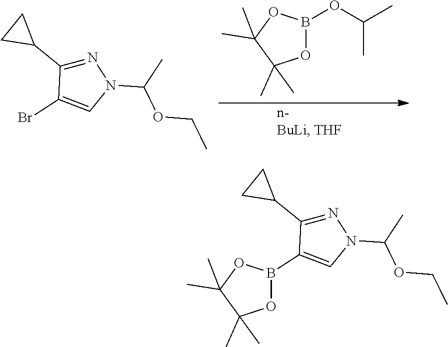 [SMILES: CCOC(C)n1cc(Br)c(C2CC2)n1 ; CC(C)OB1OC(C)(C)C(C)(C)O1 ; CCOC(C)n1cc(B2OC(C)(C)C(C)(C)O2)c(C2CC2)n1]

To a solution of 4-bromo-3-cyclopropyl-1-(1-ethoxyethyl)-1H-pyrazole (7 g, 27.01 mmol) in THF (70 mL) was added N-butyllithium (2.5 M in hexane, 14 mL, 35.12 mmol) at −70° C. under N₂. The mixture was stirred for 1 h, before 2-isopropoxy-4,4,5,5-tetramethyl-1,3,2-dioxaborolane (7.54 g, 40.52 mmol) was added. After the obtained mixture was stirred for 30 min, aqueous NH₄Cl solution (70 mL) and EtOAc (70 mL) was added. The mixture was stirred for another 30 min. The organic phase was separated, dried, and concentrated to provide the title compound (8.1 g, yield: 98%) as oil. MS (ESI) m/z=307.3 [M+H]⁺.

Step 4. Synthesis of 3-chloro-2-(3-cyclopropyl-1-(1-ethoxyethyl)-1H-pyrazol-4-yl)pyridine

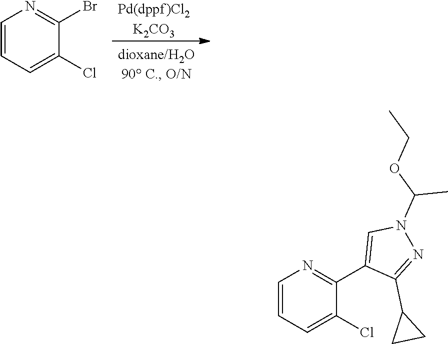 [SMILES: Clc1cccnc1Br ; CCOC(C)n1cc(-c2ncccc2Cl)c(C2CC2)n1]

To a mixture of 2-bromo-3-chloro-pyridine (1.2 g, 6.24 mmol) and 3-cyclopropyl-1-(1-ethoxyethyl)-4-(4,4,5,5-tetramethyl-1,3,2-dioxaborolan-2-yl)-1H-pyrazole (1.9 g, 6.2 mmol) in water (16 mL) and dioxane (2 mL) were added Pd(dppf)Cl₂ (455 mg, 623 μmol) and K₂CO₃ (1.7 g, 12.5 mmol). The reaction mixture was stirred at 90° C. for 6 h, before it was quenched with water. The obtained mixture was extracted with EtOAc. The organic phase was washed with water, dried over Na₂SO₄, filtered, and concentrated to provide the title compound (1.7 g, yield: 93%) as brown oil. MS (ESI) m/z=292.2 [M+H]⁺.

Step 5. Synthesis of 3-chloro-2-(3-cyclopropyl-1H-pyrazol-4-yl)pyridine

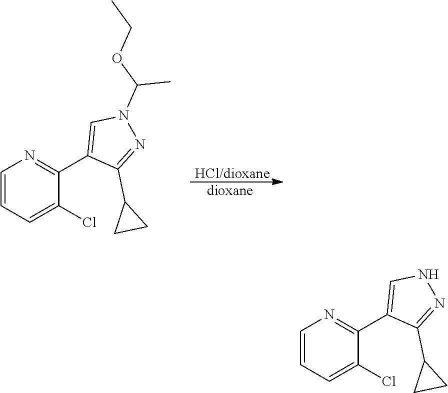 [SMILES: CCOC(C)n1cc(-c2ncccc2Cl)c(C2CC2)n1 ; Clc1cccnc1-c1c[nH]nc1C1CC1]

To a stirred solution of 3-chloro-2-(3-cyclopropyl-1-(1-ethoxyethyl)-1H-pyrazol-4-yl)pyridine (1.7 g, 5.8 mmol) in dioxane (4 mL) was added HCl solution (4 M in dioxane, 4 mL). The reaction mixture was stirred at rt for 10 min, before it was filtered. The filtrate was discarded, and the filter cake was further purified by reverse phase chromatography (0-70% MeCN in H₂O) to provide the title compound (1.0 g, yield: 78%) as bright oil. MS (ESI) m/z=220.2 [M+H]⁺.

Step 6 to 8. Synthesis of 5-((3-(trans-3-(4-(3-chloropyridin-2-yl)-3-cyclopropyl-1H-pyrazol-1-yl)cyclobutyl)propyl)amino)-2-(2,6-dioxopiperidin-3-yl)isoindoline-1,3-dione

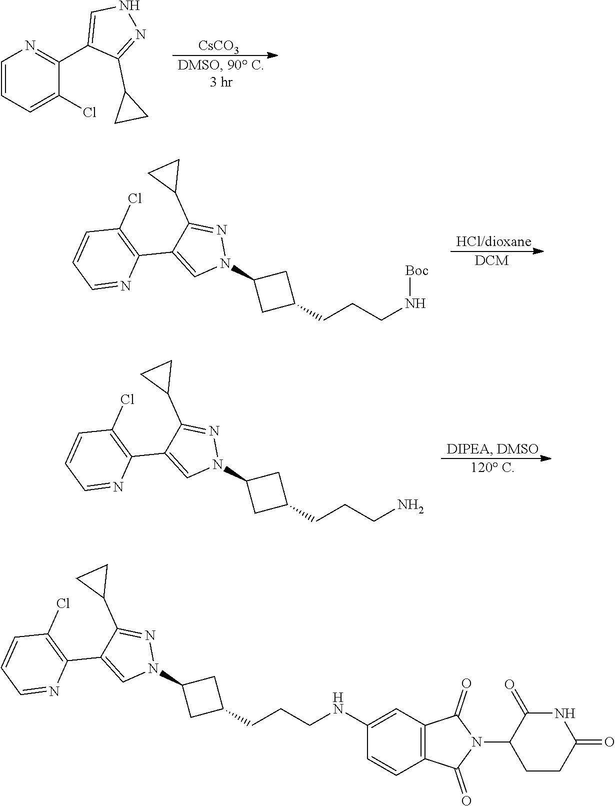

GS-070 was synthesized following the similar procedures as described for steps 10 to 11 of GS-311 (167 mg) as yellow solid. MS (ESI) m/z=587.7 [M+H]⁺.

### Example 575. 2-(2,6-Dioxopiperidin-3-yl)-5-(((trans-3-(4-(6-methylpyridin-2-yl)-3-(tetrahydro-2H-pyran-4-yl)-1H-pyrazol-1-yl)cyclobutyl)methyl)amino)isoindoline-1,3-dione (GS-071)

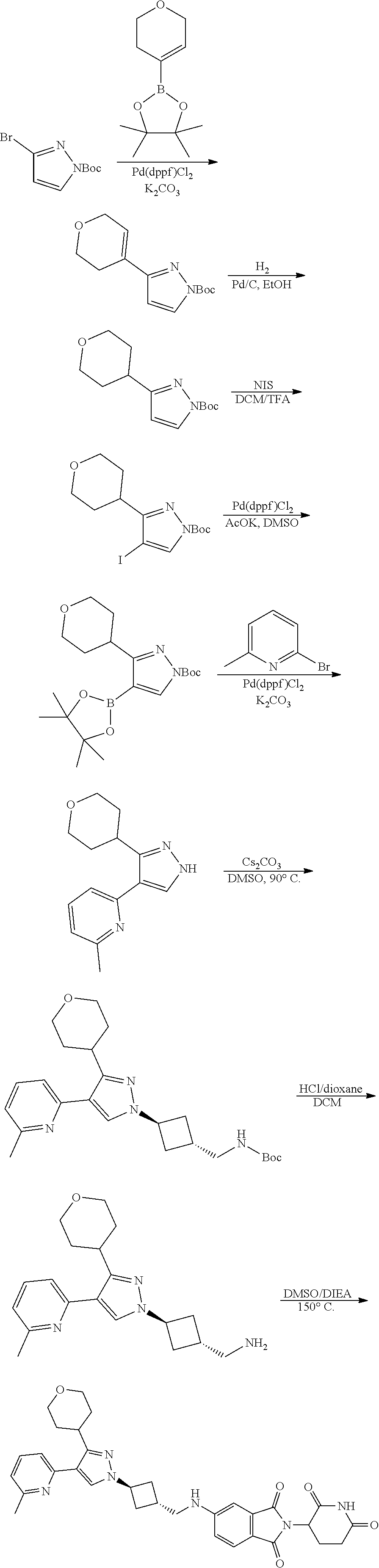

Step 1. Synthesis of tert-butyl 3-(3,6-dihydro-2H-pyran-4-yl)pyrazole-1-carboxylate

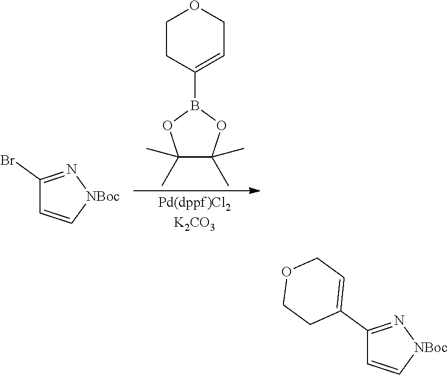

A mixture of tert-butyl 3-bromopyrazole-1-carboxylate (2.2 g, 8.90 mmol), 2-(3,6-dihydro-2H-pyran-4-yl)-4,4,5,5-tetramethyl-1,3,2-dioxaborolane (2.5 g, 11.90 mmol), K₂CO₃ (2.46 g, 17.81 mmol) and Pd(dppf)Cl₂ (200 mg, 8.90 mmol) in dioxane (15 mL) and water (3 mL) was heated at 90° C. under N₂ for 1 h. The mixture was cooled to rt, poured to water (50 mL), and extracted with EtOAc (40 mL) twice. The combined organic layers were washed with brine (30 mL), dried and concentrated. The resulting residue was purified by silica gel chromatography to provide the title compound (1.4 g, yield: 57%) as colorless oil. MS (ESI) m/z=151.1 [M+H−100]⁺.

Step 2. Synthesis of tert-butyl 3-tetrahydropyran-4-ylpyrazole-1-carboxylate

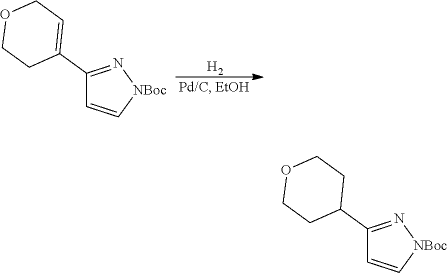 [SMILES: CC(C)(C)OC(=O)n1ccc(C2=CCOCC2)n1 ; "H2" ; CC(C)(C)OC(=O)n1ccc(C2CCOCC2)n1]

To a solution of tert-butyl 3-(3,6-dihydro-2H-pyran-4-yl)pyrazole-1-carboxylate (1.4 g, 5.03 mmol) in ethanol (50 mL) was added Pd/C (300 mg) at rt. The mixture was stirred at rt overnight under H₂. The mixture was filtered. The filtrate was concentrated to provide the title compound (1.25 g, yield: 95%) as colorless oil. MS (ESI) m/z=153.1 [M+H−100]⁺.

Step 3. Synthesis of tert-butyl 4-iodo-3-tetrahydropyran-4-yl-pyrazole-1-carboxylate

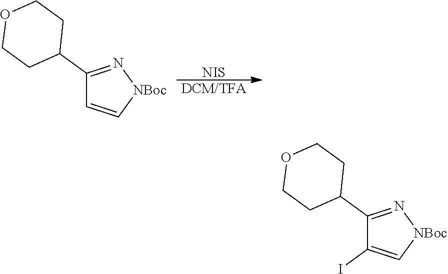 [SMILES: CC(C)(C)OC(=O)n1ccc(C2CCOCC2)n1 ; "NIS" ; CC(C)(C)OC(=O)n1cc(I)c(C2CCOCC2)n1]

To a solution of tert-butyl 3-tetrahydropyran-4-ylpyrazole-1-carboxylate (350 mg, 1.35 mmol) in DCM (10 mL) were added TFA (0.2 mL) and 1-iodopyrrolidine-2,5-dione (363 mg, 1.61 mmol) at rt. The mixture was stirred for 2 h. The mixture was diluted with DCM (50 mL), washed with sat. Na₂CO₃ solution (10 mL), sat. Na₂SO₃ solution (10 mL) and brine (30 mL). The organic phase was dried and concentrated. The resulting residue was purified by silica column to provide the title compound (200 mg, yield: 39%) as colorless oil. MS (ESI) m/z=279.0 [M+H−100]⁺.

Step 4. Synthesis of tert-butyl 3-tetrahydropyran-4-yl-4-(4,4,5,5-tetramethyl-1,3,2-dioxaborolan-2-yl)pyrazole-1-carboxylate

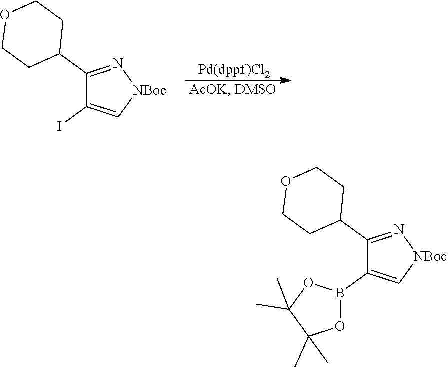 [SMILES: CC(C)(C)OC(=O)n1cc(I)c(C2CCOCC2)n1 ; CC(C)(C)OC(=O)n1cc(B2OC(C)(C)C(C)(C)O2)c(C2CCOCC2)n1]

A mixture of tert-butyl 4-iodo-3-tetrahydropyran-4-yl-pyrazole-1-carboxylate (650 mg, 1.55 mmol), 4,4,5,5-tetramethyl-2-(4,4,5,5-tetramethyl-1,3,2-dioxaborolan-2-yl)-1,3,2-dioxaborolane (785 mg, 3.09 mmol), acetoxypotassium (606 mg, 6.17 mmol) and Pd(dppf)Cl₂ (60 mg, 1.55 mmol) in DMSO (5.00 mL) was heated at 80° C. for 2 h. The mixture was cooled to rt, poured to water (50 mL), and extracted with EtOAc (50 mL) twice. The combined organic phases were dried and concentrated to provide the title compound (580 mg, yield: 89%) as brown oil. MS (ESI) m/z=279.0 [M+H−100]⁺.

Step 5. Synthesis of 2-methyl-6-(5-tetrahydropyran-4-yl-1H-pyrazol-4-yl)pyridine

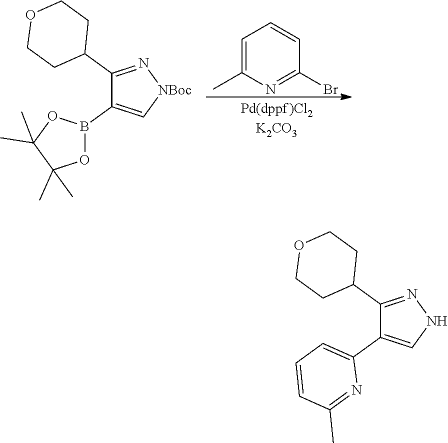

A mixture of tert-butyl 3-tetrahydropyran-4-yl-4-(4,4,5,5-tetramethyl-1,3,2-dioxaborolan-2-yl)pyrazole-1-carboxylate (580 mg, 1.53 mmol), 2-bromo-6-methyl-pyridine (263.76 mg, 1.53 mmol), Pd(dppf)Cl₂ (50 mg, 1.53 mmol) and K₂CO₃ (425 mg, 3.08 mmol) in dioxane (10 mL) and water (1 mL) was heated at 90° C. for 1 h. The mixture was cooled to rt, and concentrated. The resulting residue was dissolved in methanol (10 mL). To this solution was added aq. NaOH solution (30%, 0.3 mL). The mixture was heated at 70° C. for 1 h. The mixture was cooled to rt, and concentrated. The resulting residue was diluted with DCM (60 mL), washed with brine (30 mL), dried and concentrated. The resulting residue was purified by reverse phase chromatography to provide the title compound (50 mg, yield: 13%) as light brown oil. MS (ESI) m/z=244.2 [M+H]⁺.

Step 6. Synthesis of (trans-3-(4-(6-methylpyridin-2-yl)-3-(tetrahydro-2H-pyran-4-yl)-1H-pyrazol-1-yl)cyclobutyl)methanamine

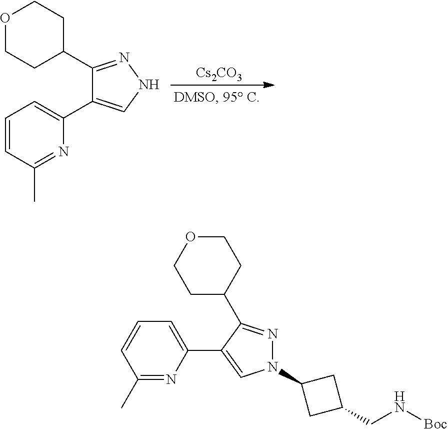

To a mixture of 2-methyl-6-(3-tetrahydropyran-4-yl-1H-pyrazol-4-yl)pyridine (50 mg, 205 μmol) and [3-[[bis(tert-butoxycarbonyl)amino]methyl]cyclobutyl] 4-methylbenzenesulfonate (140 mg, 308 μmol) in DMSO (2 mL) was added Cs₂CO₃ (134 mg, 411 μmol). The reaction was stirred at 90° C. for 4 h. The solution was quenched with water, and extracted with EtOAc. The organic layer was washed with water and brine, dried over Na₂SO₄, filtered, and concentrated. The resulting residue was purified by reverse phase chromatography (0-70% MeCN in H₂O) to provide the title compound (20 mg, yield: 23%) as a bright oil. MS (ESI) m/z=427.4 [M+H]⁺.

Step 7. Synthesis of (trans-3-(4-(6-methylpyridin-2-yl)-3-(tetrahydro-2H-pyran-4-yl)-1H-pyrazol-1-yl)cyclobutyl)methanamine

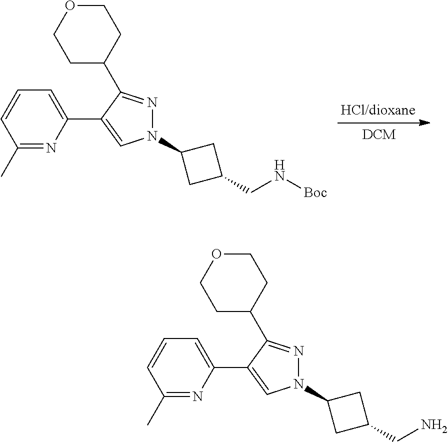 [SMILES: Cc1cccc(-c2cn([C@H]3C[C@H](CNC(=O)OC(C)(C)C)C3)nc2C2CCOCC2)n1 ; Cc1cccc(-c2cn([C@H]3C[C@H](CN)C3)nc2C2CCOCC2)n1]

To a solution of tert-butyl N-[[3-[4-(6-methyl-2-pyridyl)-3-tetrahydropyran-4-yl-pyrazol-1-yl]cyclobutyl]methyl]carbamate (20 mg, 46.89 μmol) in DCM (0.5 mL) was added HCl/dioxane (0.5 mL). The reaction was stirred at rt for 10 min. The mixture was concentrated to provide [3-[4-(6-methyl-2-pyridyl)-3-tetrahydropyran-4-yl-pyrazol-1-yl]cyclobutyl]methanamine (13 mg, yield: 84.9%) as a white solid. MS (ESI) m/z=327.4[M+H]⁺.

Step 8. Synthesis of 2-(2,6-dioxopiperidin-3-yl)-5-(((trans-3-(4-(6-methylpyridin-2-yl)-3-(tetrahydro-2H-pyran-4-yl)-1H-pyrazol-1-yl)cyclobutyl)methyl)amino)isoindoline-1,3-dione

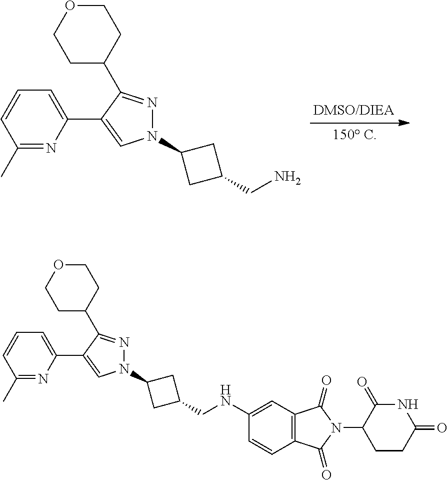 [SMILES: Cc1cccc(-c2cn([C@H]3C[C@H](CN)C3)nc2C2CCOCC2)n1 ; Cc1cccc(-c2cn([C@H]3C[C@H](CNc4ccc5c(c4)C(=O)N(C4CCC(=O)NC4=O)C5=O)C3)nc2C2CCOCC2)n1]

To a mixture of (trans-3-(4-(6-methylpyridin-2-yl)-3-(tetrahydro-2H-pyran-4-yl)-1H-pyrazol-1-yl)cyclobutyl)methanamine (13 mg, 40 μmol) and 2-(2,6-dioxo-3-piperidyl)-5-fluoro-isoindoline-1,3-dione (16 mg, 60 μmol) in DMSO (1 mL) was added DIPEA (15 mg, 119 μmol). The reaction mixture was stirred at 150° C. for 30 min. The reaction was purified by reverse phase chromatography (0-70% MeCN in H₂O) to provide the title compound (0.3 mg, yield: 1%) as yellow solid. MS (ESI) m/z=583.8 [M+H]⁺.

### Example 576. 5-(4-(4-(8-Bromoquinoxalin-2-yl)-1H-pyrazol-1-yl)piperidin-1-yl)-N-((2-(2,6-dioxopiperidin-3-yl)-1-oxoisoindolin-5-yl)methyl)-4,4-difluoro-5-oxopentanamide (GS-054)

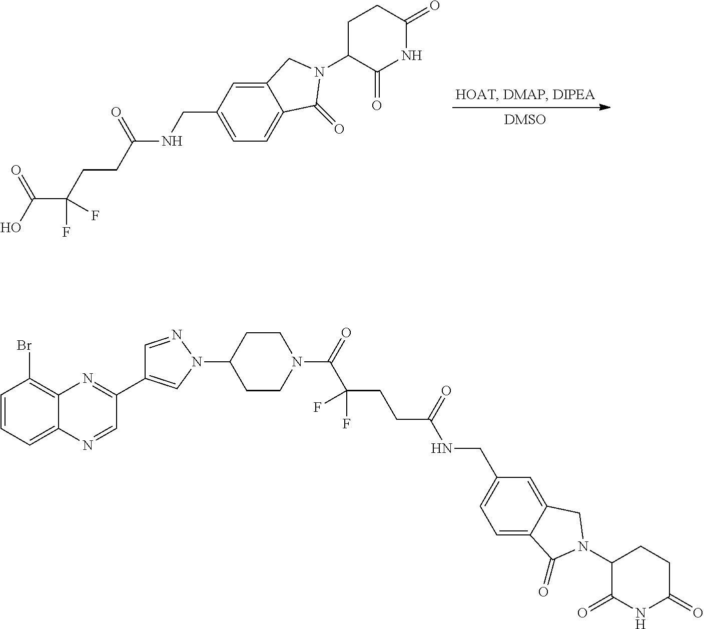 [SMILES: O=C(CCC(F)(F)C(=O)O)NCc1ccc2c(c1)CN(C1CCC(=O)NC1=O)C2=O ; O=C(CCC(F)(F)C(=O)N1CCC(n2cc(-c3cnc4cccc(Br)c4n3)cn2)CC1)NCc1ccc2c(c1)CN(C1CCC(=O)NC1=O)C2=O]

GS-054 was synthesized following the similar procedures as described for GS-518 (5.1 mg, yield: 57%) as yellow solid. MS (ESI) m/z=765.4 [M+H]⁺.

### Example 577. 5-((6-(4-(4-Cyclohexyl-1H-pyrazol-1-yl)piperidin-1-yl)-6-oxohexyl)amino)-2-(2,6-dioxopiperidin-3-yl)isoindoline-1,3-dione (GS-049)

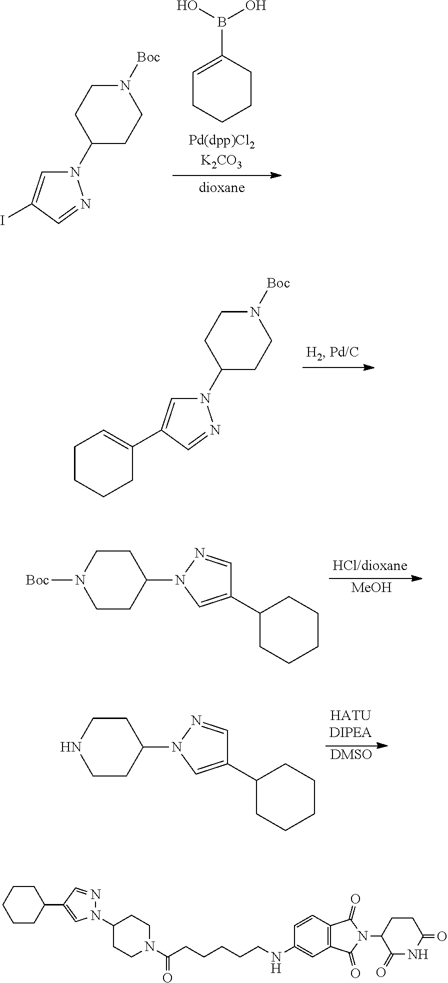

Step 1. Synthesis of tert-butyl 4-(4-(cyclohex-1-en-1-yl)-1H-pyrazol-1-yl)piperidine-1-carboxylate

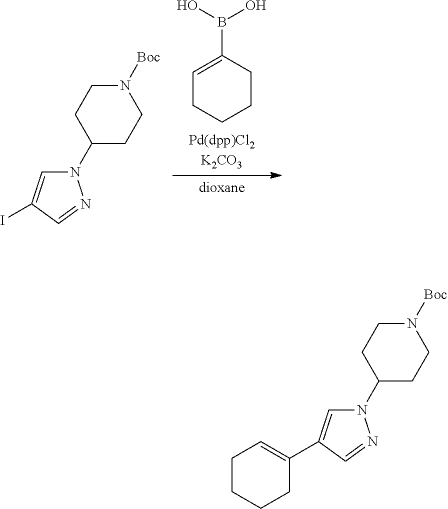 [SMILES: CC(C)(C)OC(=O)N1CCC(n2cc(I)cn2)CC1 ; OB(O)C1=CCCCC1 ; CC(C)(C)OC(=O)N1CCC(n2cc(C3=CCCCC3)cn2)CC1]

To a mixture of tert-butyl 4-(4-iodopyrazol-1-yl)piperidine-1-carboxylate (100 mg, 265 μmol) and cyclohexen-1-ylboronic acid (67 mg, 530 μmol) in dioxane (2 mL) were added K₂CO₃ (37 mg, 265 μmol) and Pd(dppf)Cl₂ (194 mg, 265 μmol). The reaction mixture was stirred at 80° C. overnight. The solution was quenched with water, and extracted with EtOAc. The organic layer was washed with brine, dried over Na₂SO₄, filtered, and concentrated. The resulting residue was purified by silica gel chromatography (petroleum ether:EtOAc=1:0 to 3:2) to provide the title compound (50 mg, yield: 57%) as light-yellow solid. MS (ESI) m/z=332.4 [M+H]⁺.

Step 2. Synthesis of tert-butyl 4-(4-cyclohexyl-TH-pyrazol-1-yl)piperidine-1-carboxylate

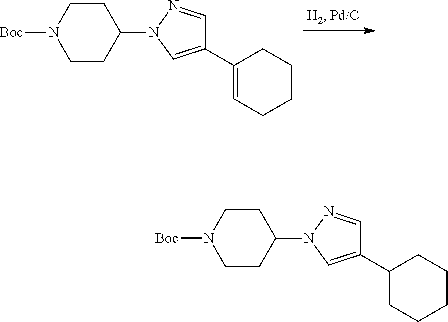 [SMILES: CC(C)(C)OC(=O)N1CCC(n2cc(C3=CCCCC3)cn2)CC1 ; CC(C)(C)OC(=O)N1CCC(n2cc(C3CCCCC3)cn2)CC1]

To a solution of tert-butyl 4-(4-(cyclohex-1-en-1-yl)-1H-pyrazol-1-yl)piperidine-1-carboxylate (50 mg, 150.85 μmol) in methanol (2 mL) was added Pd/C (10 mg, 82 μmol). The reaction mixture was stirred at rt for 3 h, before it was filtered. The filtrate was concentrated to provide the title compound (30 mg, yield: 60%) as bright oil, which was used directly in the next step. MS (ESI) m/z=334.6 [M+H]⁺.

Step 3 Synthesis of 4-(4-cyclohexyl-1H-pyrazol-1-yl)piperidine

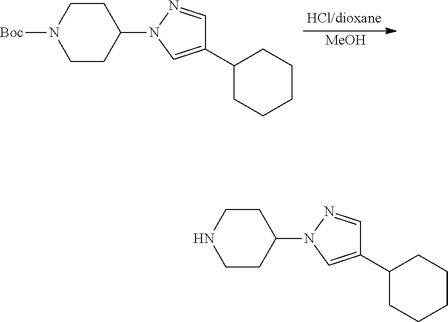 [SMILES: "MeOH" ; CC(C)(C)OC(=O)N1CCC(n2cc(C3CCCCC3)cn2)CC1 ; c1nn(C2CCNCC2)cc1C1CCCCC1]

To a solution of tert-butyl 4-(4-cyclohexyl-1H-pyrazol-1-yl)piperidine-1-carboxylate (30 mg, 90 μmol) in methanol (0.5 mL) was added HCl solution (4 M in dioxane, 0.5 mL). The reaction mixture was stirred at rt for 30 min, before it was concentrated to provide the title compound (30 mg, yield: 86%), which was used directly in the next step. MS (ESI) m/z=234.5 [M+H]⁺.

Step 4. Synthesis of 5-((6-(4-(4-cyclohexyl-1H-pyrazol-1-yl)piperidin-1-yl)-6-oxohexyl)amino)-2-(2,6-dioxopiperidin-3-yl)isoindoline-1,3-dione

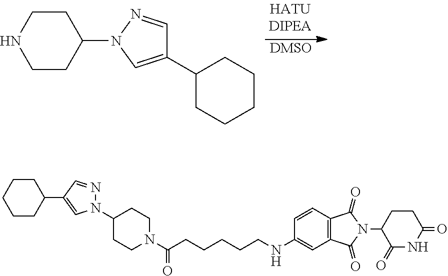

To a mixture of 6-((2-(2,6-dioxopiperidin-3-yl)-1,3-dioxoisoindolin-5-yl)amino)hexanoic acid (30 mg, 77 μmol) and 4-(4-cyclohexyl-1H-pyrazol-1-yl)piperidine (18 mg, 77 μmol) in DMSO (2 mL) were added HATU (59 mg, 155 μmol) and DIPEA (40 mg, 310 μmol). The reaction mixture was stirred at rt for 1 h. The reaction was purified by reverse phase chromatography (0-70% MeCN in H₂O) to provide the title compound (8.4 mg, yield: 18%) as yellow solid. MS (ESI) m/z=603.7 [M+H]⁺.

### Example 578. 2-(2,6-Dioxopiperidin-3-yl)-5-((6-oxo-6-(4-(4-(pyridin-2-yl)-1H-pyrazol-1-yl)piperidin-1-yl)hexyl)amino)isoindoline-1,3-dione (GS-053)

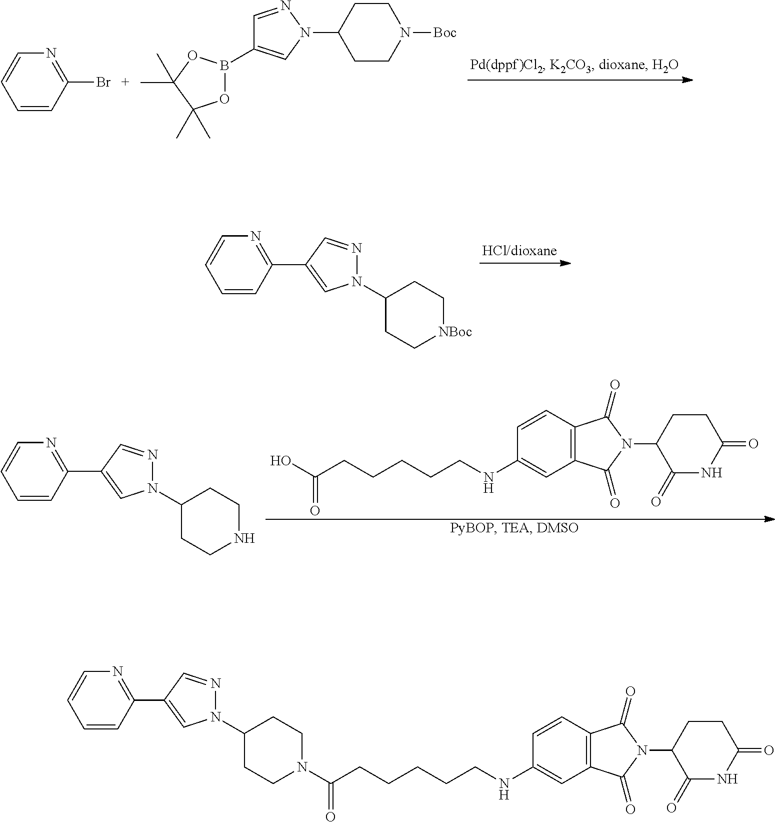

GS-053 was synthesized following the similar procedures as described for GS-005 (16.1 mg). MS (ESI) m/z=598.6 [M+H]⁺.

### Example 579. 2-(2,6-Dioxopiperidin-3-yl)-5-((6-oxo-6-(4-(4-(pyrimidin-2-yl)-1H-pyrazol-1-yl)piperidin-1-yl)hexyl)amino)isoindoline-1,3-dione (GS-051)

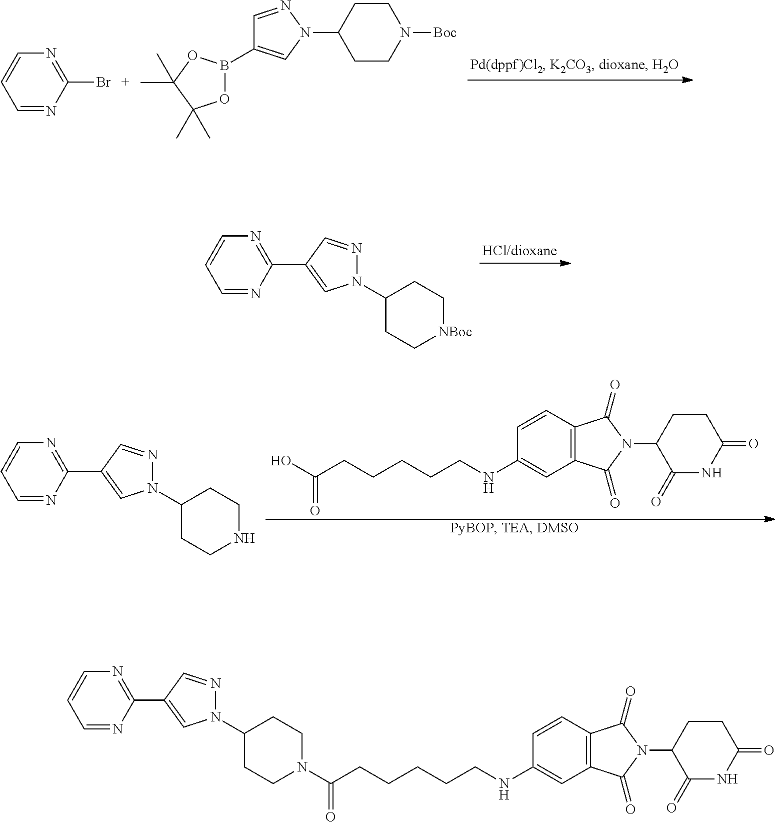

GS-051 was synthesized following the similar procedures as described for GS-005 (27.5 mg). MS (ESI) m/z=599.6 [M+H]⁺.

### Example 580. 2-(2,6-Dioxopiperidin-3-yl)-5-((6-oxo-6-(4-(4-phenyl-1H-pyrazol-1-yl)piperidin-1-yl)hexyl)amino)isoindoline-1,3-dione (GS-050)

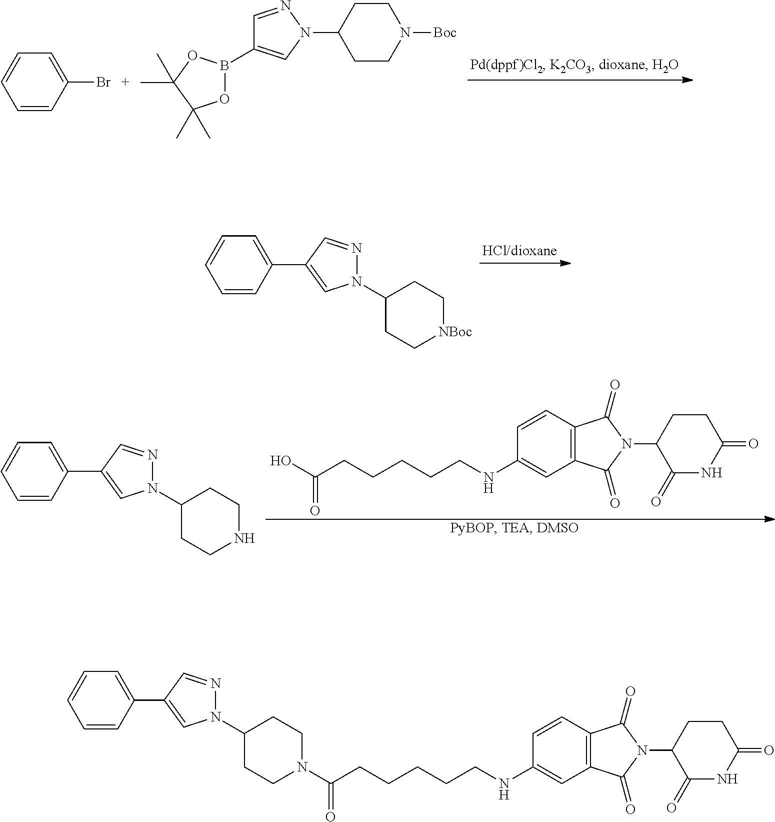

GS-050 was synthesized following the similar procedures as described for GS-005 (23.2 mg). MS (ESI) m/z=597.6 [M+H]⁺.

### Example 581. 5-((6-(4-(4-(8-Bromoquinolin-2-yl)-1H-pyrazol-1-yl)piperidin-1-yl)-6-oxohexyl)amino)-2-(2,6-dioxopiperidin-3-yl)isoindoline-1,3-dione (GS-052)

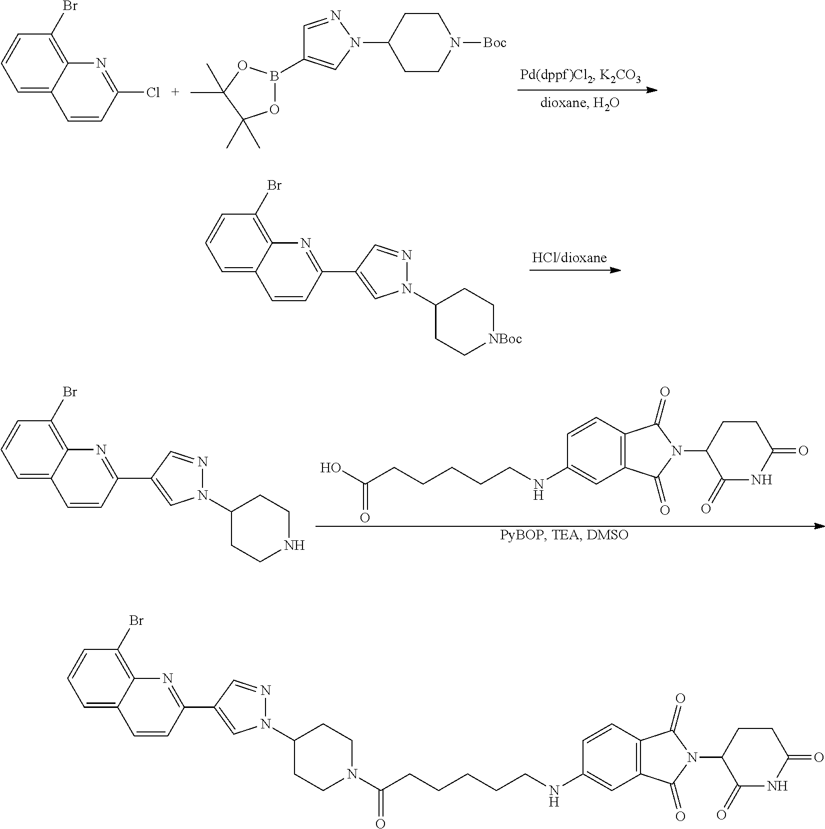

GS-052 was synthesized following the similar procedures as described for GS-005 (13.4 mg). MS (ESI) m/z=726.5 [M+H]⁺.

### Example 582. 2-(2,6-Dioxopiperidin-3-yl)-5-((6-oxo-6-(4-(4-(8-(piperazin-1-yl)quinoxalin-2-yl)-1H-pyrazol-1-yl)piperidin-1-yl)hexyl)amino)isoindoline-1,3-dione (GS-035)

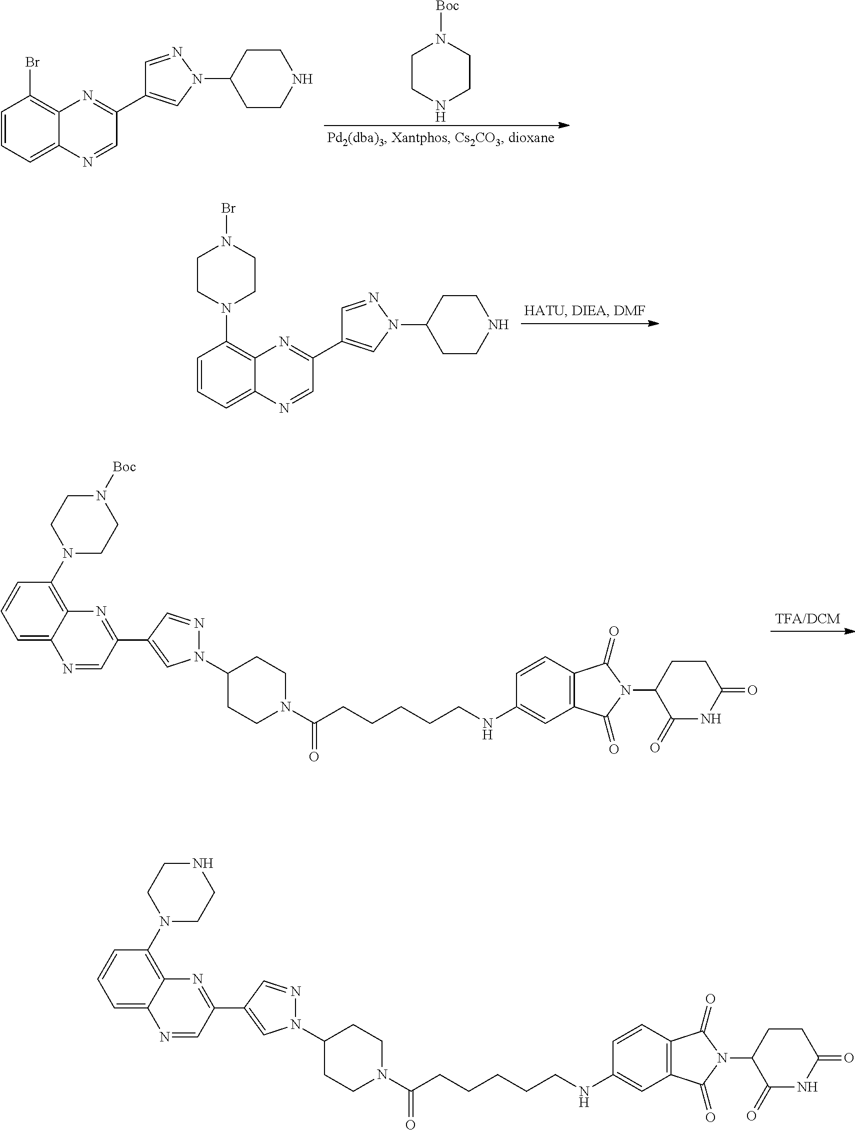 [SMILES: Brc1cccc2ncc(-c3cnn(C4CCNCC4)c3)nc12 ; CC(C)(C)OC(=O)N1CCNCC1 ; BrN1CCN(c2cccc3ncc(-c4cnn(C5CCNCC5)c4)nc23)CC1 ; CC(C)(C)OC(=O)N1CCN(c2cccc3ncc(-c4cnn(C5CCN(C(=O)CCCCCNc6ccc7c(c6)C(=O)N(C6CCC(=O)NC6=O)C7=O)CC5)c4)nc23)CC1 ; O=C1CCC(N2C(=O)c3ccc(NCCCCCC(=O)N4CCC(n5cc(-c6cnc7cccc(N8CCNCC8)c7n6)cn5)CC4)cc3C2=O)C(=O)N1]

Step 1. Synthesis of tert-butyl 4-(3-(1-(piperidin-4-yl)-1H-pyrazol-4-yl)quinoxalin-5-yl)piperazine-1-carboxylate

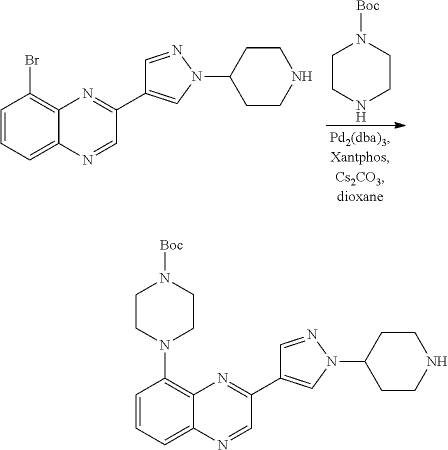

A mixture of 8-bromo-2-(1-(piperidin-4-yl)-1H-pyrazol-4-yl)quinoxaline (see step 2 of GS-044 for synthesis, 70 mg, 148.22 μmol, TFA salt), tert-butyl piperazine-1-carboxylate (220 mg, 1.18 mmol), cesium carbonate (100 mg, 306.92 μmol), Pa₂(dba)₃ (20 mg) and xantphos (20 mg) in dioxane (2 mL) was heated at 100° C. for 2 h under N₂. The mixture was cooled to rt and concentrated. The resulting residue was purified by reverse phase chromatography to provide the title compound (20 mg, yield: 27%) as yellow solid. MS (ESI) m/z=464.5 [M+H]⁺.

Step 2. Synthesis of tert-butyl 4-(3-(1-(1-(6-((2-(2,6-dioxopiperidin-3-yl)-1,3-dioxoisoindolin-5-yl)amino)hexanoyl)piperidin-4-yl)-1H-pyrazol-4-yl)quinoxalin-5-yl)piperazine-1-carboxylate

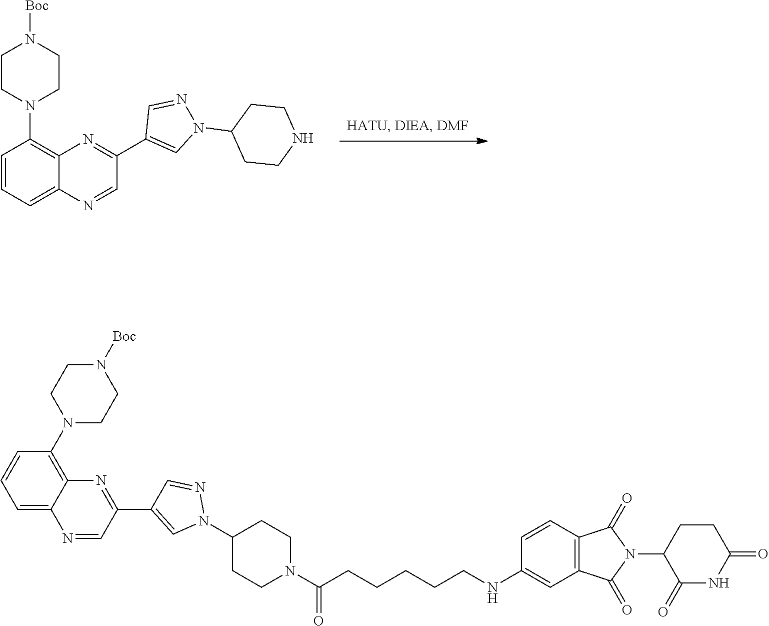 [SMILES: CC(C)(C)OC(=O)N1CCN(c2cccc3ncc(-c4cnn(C5CCNCC5)c4)nc23)CC1 ; CC(C)(C)OC(=O)N1CCN(c2cccc3ncc(-c4cnn(C5CCN(C(=O)CCCCCNc6ccc7c(c6)C(=O)N(C6CCC(=O)NC6=O)C7=O)CC5)c4)nc23)CC1]

A mixture of tert-butyl 4-(3-(1-(piperidin-4-yl)-1H-pyrazol-4-yl)quinoxalin-5-yl)piperazine-1-carboxylate (20 mg, 39.69 μmol), 6-((2-(2,6-dioxopiperidin-3-yl)-1,3-dioxoisoindolin-5-yl)amino)hexanoic acid (20 mg, 51.63 μmol), HATU (65 mg, 79.50 μmol) and DIPEA (50 mg, 386.87 μmol) in DMF (2 mL) was stirred at rt overnight. The mixture was purified by reverse phase chromatography to provide the title compound (15 mg, yield: 43%) as yellow solid. MS (ESI) m/z=833.9 [M+H]⁺.

Step 3. Synthesis of 2-(2,6-dioxopiperidin-3-yl)-5-((6-oxo-6-(4-(4-(8-(piperazin-1-yl)quinoxalin-2-yl)-1H-pyrazol-1-yl)piperidin-1-yl)hexyl)amino)isoindoline-1,3-dione

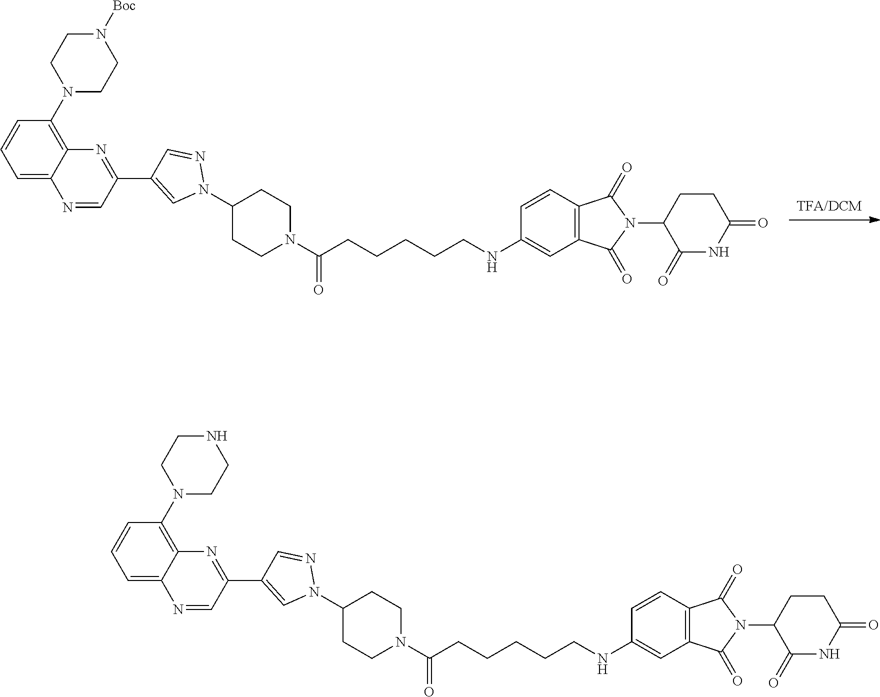 [SMILES: CC(C)(C)OC(=O)N1CCN(c2cccc3ncc(-c4cnn(C5CCN(C(=O)CCCCCNc6ccc7c(c6)C(=O)N(C6CCC(=O)NC6=O)C7=O)CC5)c4)nc23)CC1 ; O=C1CCC(N2C(=O)c3ccc(NCCCCCC(=O)N4CCC(n5cc(-c6cnc7cccc(N8CCNCC8)c7n6)cn5)CC4)cc3C2=O)C(=O)N1]

To a solution of tert-butyl 4-(3-(1-(1-(6-((2-(2,6-dioxopiperidin-3-yl)-1,3-dioxoisoindolin-5-yl)amino)hexanoyl)piperidin-4-yl)-1H-pyrazol-4-yl)quinoxalin-5-yl)piperazine-1-carboxylate (15 mg, 17.11 μmol) in DCM (3 mL) was added TFA (1.5 mL). The mixture was stirred at rt for 0.5 h, before it was concentrated. The resulting residue was purified by reverse phase chromatography to provide the title compound (5 mg, yield: 40%) as yellow solid. MS (ESI) m % z=733.7 [M+H]⁺.

### Example 583. 2-(2,6-Dioxopiperidin-3-yl)-5-((6-(4-(4-(8-morpholinoquinoxalin-2-yl)-1H-pyrazol-1-yl)piperidin-1-yl)-6-oxohexyl)amino)isoindoline-1,3-dione (GS-034)

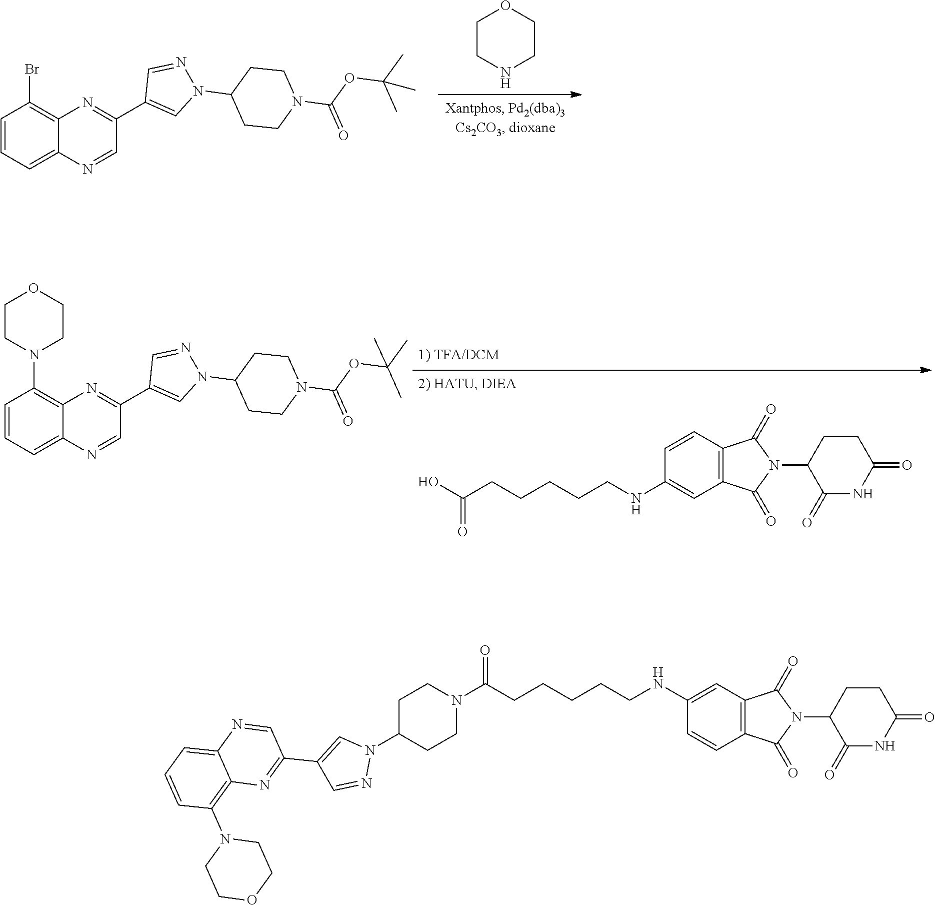 [SMILES: CC(C)(C)OC(=O)N1CCC(n2cc(-c3cnc4cccc(Br)c4n3)cn2)CC1 ; C1COCCN1 ; CC(C)(C)OC(=O)N1CCC(n2cc(-c3cnc4cccc(N5CCOCC5)c4n3)cn2)CC1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(n5cc(-c6cnc7cccc(N8CCOCC8)c7n6)cn5)CC4)cc3C2=O)C(=O)N1]

Step 1. Synthesis of tert-butyl 4-(4-(8-morphohnoquinoxahn-2-yl)-1H-pyrazol-1-yl)piperidine-1-carboxylate

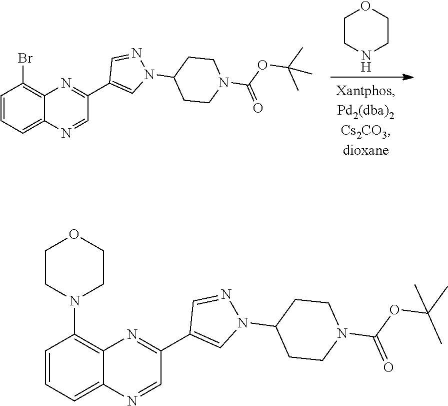 [SMILES: CC(C)(C)OC(=O)N1CCC(n2cc(-c3cnc4cccc(Br)c4n3)cn2)CC1 ; C1COCCN1 ; CC(C)(C)OC(=O)N1CCC(n2cc(-c3cnc4cccc(N5CCOCC5)c4n3)cn2)CC1]

A mixture of tert-butyl 4-(4-(8-bromoquinoxalin-2-yl)-1H-pyrazol-1-yl)piperidine-1-carboxylate (60 mg, 130.90 μmol), morpholine (150 mg, 1.72 mmol), cesium carbonate (100 mg, 306.92 μmol), xantphos (15 mg, 130.90 μmol) and Pd₂(dba)₃ (15 mg, 130.90 μmol) in dioxane (2 mL) was heated at 100° C. under N₂ for 2 h. The mixture was cooled to rt and concentrated. The resulting residue was purified by reverse phase chromatography to provide the title compound (50 mg, yield: 82%) as yellow solid. MS (ESI) m/z=465.7 [M+H]⁺.

Step 2. Synthesis of 2-(2,6-dioxopiperidin-3-yl)-5-((6-(4-(4-(8-morpholinoquinoxalin-2-yl)-1H-pyrazol-1-yl)piperidin-1-yl)-6-oxohexyl)amino)isoindoline-1,3-dione

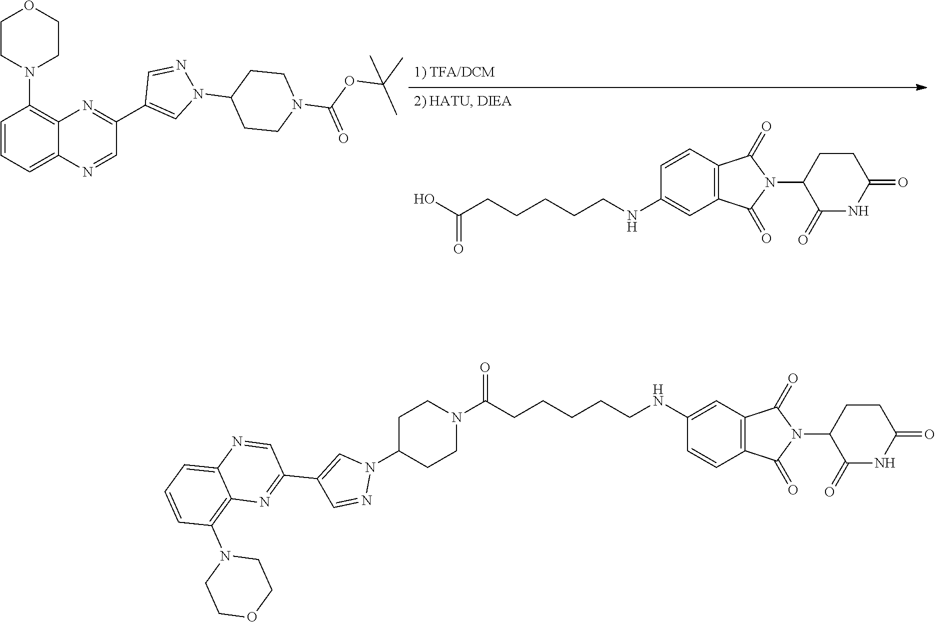 [SMILES: CC(C)(C)OC(=O)N1CCC(n2cc(-c3cnc4cccc(N5CCOCC5)c4n3)cn2)CC1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(n5cc(-c6cnc7cccc(N8CCOCC8)c7n6)cn5)CC4)cc3C2=O)C(=O)N1]

To a solution of tert-butyl 4-(4-(8-morpholinoquinoxalin-2-yl)-1H-pyrazol-1-yl)piperidine-1-carboxylate (50 mg, 107.63 μmol) in DCM (3 mL) was added TFA (1.5 mL). The reaction mixture was stirred at rt for 0.5 h. The mixture was concentrated. The resulting residue was dissolved in DMF (2 mL). To the solution were added 6-((2-(2,6-Dioxopiperidin-3-yl)-1,3-dioxoisoindolin-5-yl)amino)hexanoic acid (30 mg, 77.44 μmol), DIEA (60 mg, 464.25 μmol) and HATU (80 mg, 210.40 μmol). After the mixture was stirred at rt overnight, it was purified by reverse phase chromatography to provide the title compound (33 mg, yield: 42%) as light brown solid. MS (ESI) m/z=734.8 [M+H]⁺.

### Example 584. 2-(2,6-Dioxopiperidin-3-yl)-5-((6-(4-(4-(8-(4-methylpiperazin-1-yl)quinoxalin-2-yl)-1H-pyrazol-1-yl)piperidin-1-yl)-6-oxohexyl)amino)isoindoline-1,3-dione (GS-036)

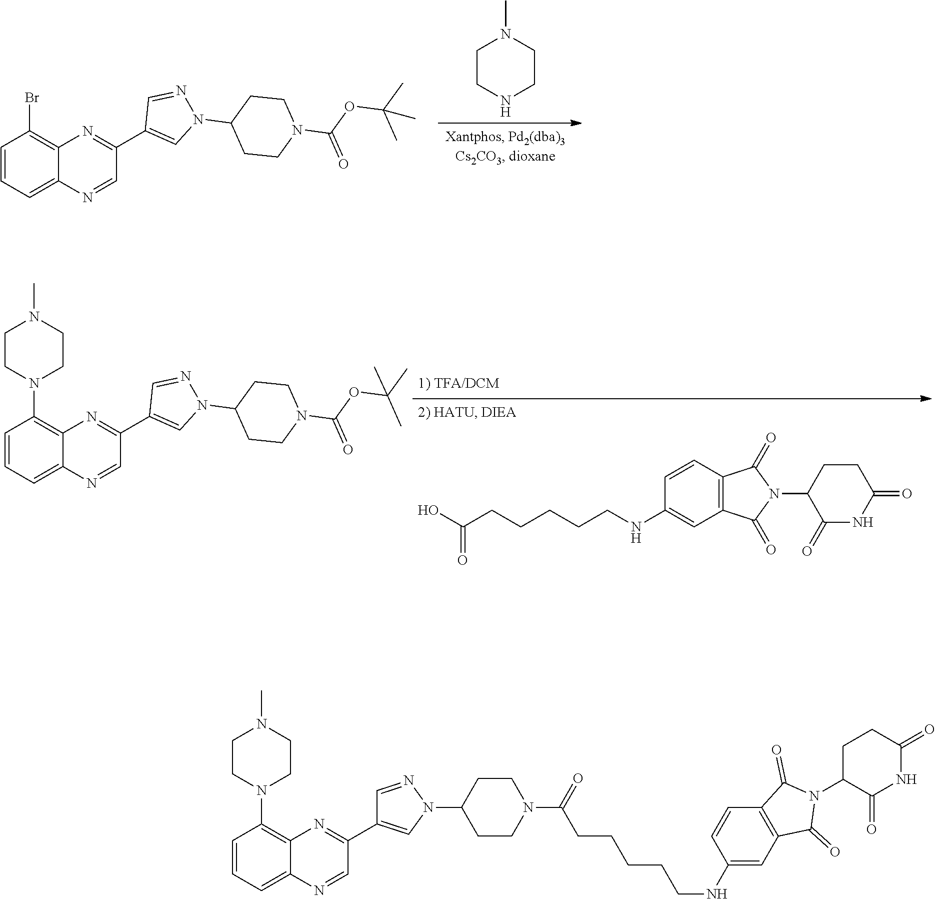

GS-036 was synthesized following the similar procedures as described for GS-034 (9 mg) as white solid. MS (ESI) m/z=547.7 [M+H]⁺

### Example 585. 2-(2,6-Dioxopiperidin-3-yl)-5-((6-(4-(4-(4-methylpiperazin-1-yl)-1H-pyrazol-1-yl)piperidin-1-yl)-6-oxohexyl)amino)isoindoline-1,3-dione (GS-046)

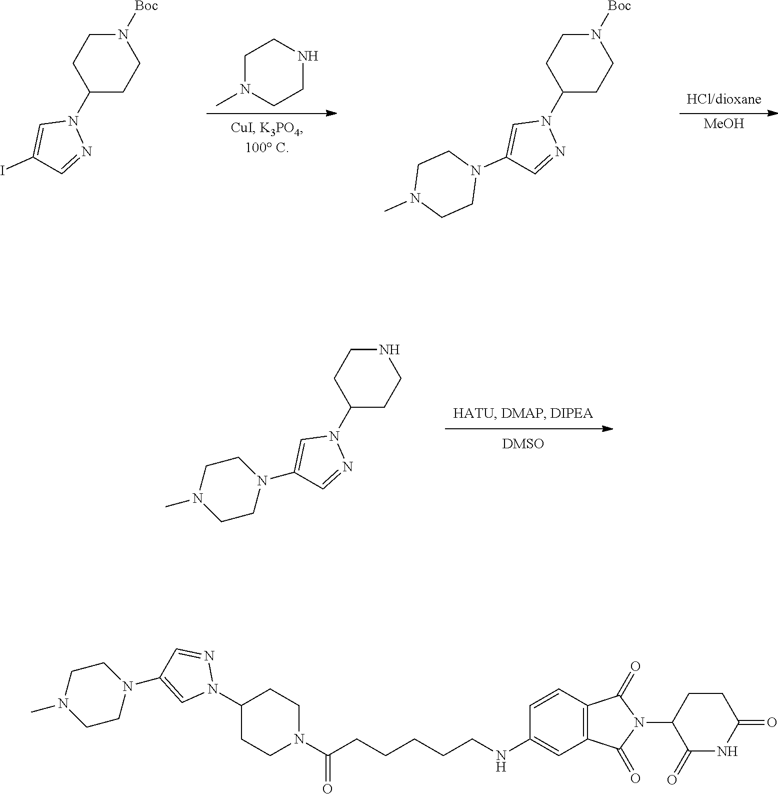

Step 1. Synthesis of tert-butyl 4-(4-(4-methylpiperazin-1-yl)-1H-pyrazol-1-yl)piperidine-1-carboxylate

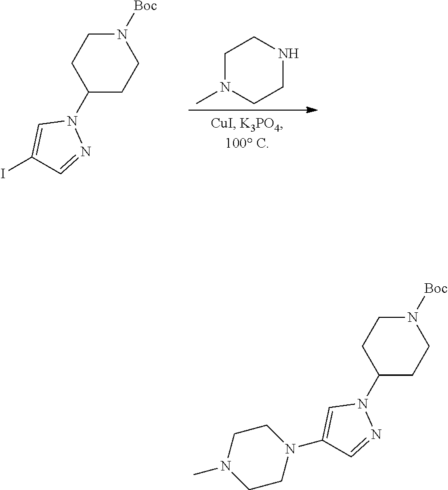

To a mixture of tert-butyl 4-(4-iodopyrazol-1-yl)piperidine-1-carboxylate (100 mg, 265 μmol) and 1-methylpiperazine (53 mg, 530 μmol) in i-PrOH (5 mL) were added CuI (101 mg, 530 μmol), K₃PO₄ (169 mg, 795 μmol) and ethylene glycol (41 mg, 663 μmol). The reaction mixture was stirred at 100° C. for 48 h. The solution was quenched with water, and extracted with EtOAc. The organic layer was washed with water and brine, dried over Na₂SO₄, filtered, and concentrated. The resulting residue was purified by prep-TLC (petroleum ether:EtOAc=2:1) to provide the title compound (17 mg, yield: 18%) as yellow solid. MS (ESI) m/z=350.6 [M+H]⁺.

Step 2. Synthesis of 1-methyl-4-(1-(piperidin-4-yl)-1H-pyrazol-4-yl)piperazine

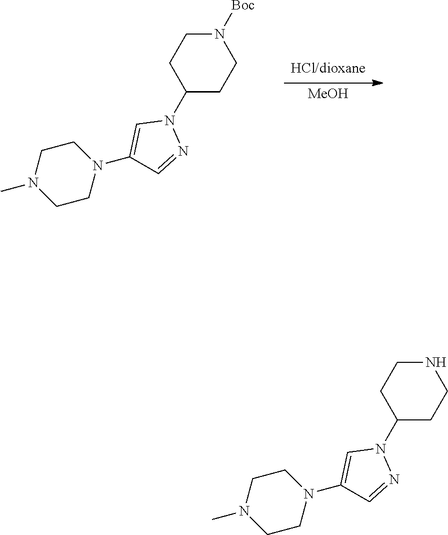 [SMILES: "MeOH" ; CN1CCN(c2cnn(C3CCN(C(=O)OC(C)(C)C)CC3)c2)CC1 ; CN1CCN(c2cnn(C3CCNCC3)c2)CC1]

To a solution of tert-butyl 4-[4-(4-methylpiperazin-1-yl)pyrazol-1-yl]piperidine-1-carboxylate (17 mg, 49 μmol) in methanol (0.2 mL) was added HCl solution (4 M in dioxane, 0.5 mL). The reaction mixture was stirred at rt for 30 min. The mixture was concentrated to provide the title compound (13 mg, crude), which was used directly in the next step. MS (ESI) m/z=250.6 [M+H]⁺.

Step 3. Synthesis of 2-(2,6-dioxopiperidin-3-yl)-5-((6-(4-(4-(4-methylpiperazin-1-yl)-JH-pyrazol-1-yl)piperidin-1-yl)-6-oxohexyl)amino)isoindoline-1,3-dione

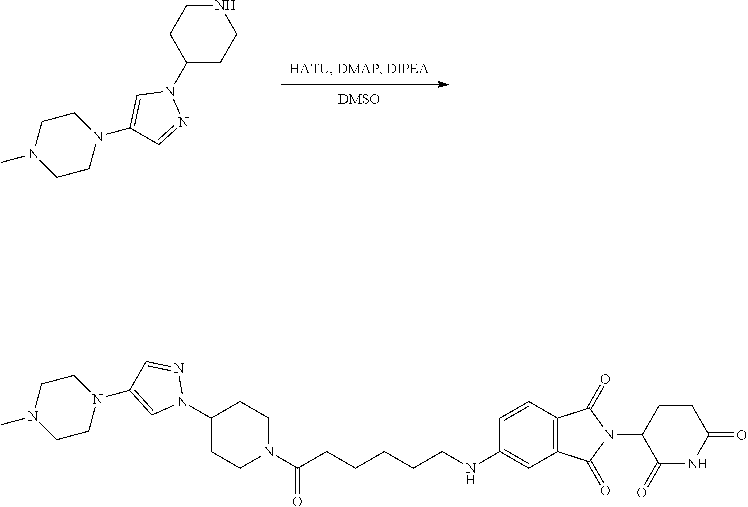 [SMILES: CN1CCN(c2cnn(C3CCNCC3)c2)CC1 ; CN1CCN(c2cnn(C3CCN(C(=O)CCCCCNc4ccc5c(c4)C(=O)N(C4CCC(=O)NC4=O)C5=O)CC3)c2)CC1]

To a mixture of 6-((2-(2,6-dioxopiperidin-3-yl)-1,3-dioxoisoindolin-5-yl)amino)hexanoic acid (10 mg, 26 μmol) and 1-methyl-4-(1-(piperidin-4-yl)-1H-pyrazol-4-yl)piperazine (13 mg, 52 μmol) in DMSO (0.5 mL) were added HATU (20 mg, 52 μmol), DMAP (315 ug, 2.6 μmol) and DIPEA (10 mg, 77 μmol). The reaction mixture was stirred at rt overnight. The reaction was purified by reverse phase chromatography (0-70% MeCN in H₂O) to provide the title compound (3.8 mg, yield: 24%) as yellow solid. MS (ESI) m/z=619.7 [M+H]⁺.

### Example 586. 2-(2,6-Dioxopiperidin-3-yl)-5-((6-oxo-6-(4-(4-(piperidin-1-yl)-1H-pyrazol-1-yl)piperidin-1-yl)hexyl)amino)isoindoline-1,3-dione (GS-048)

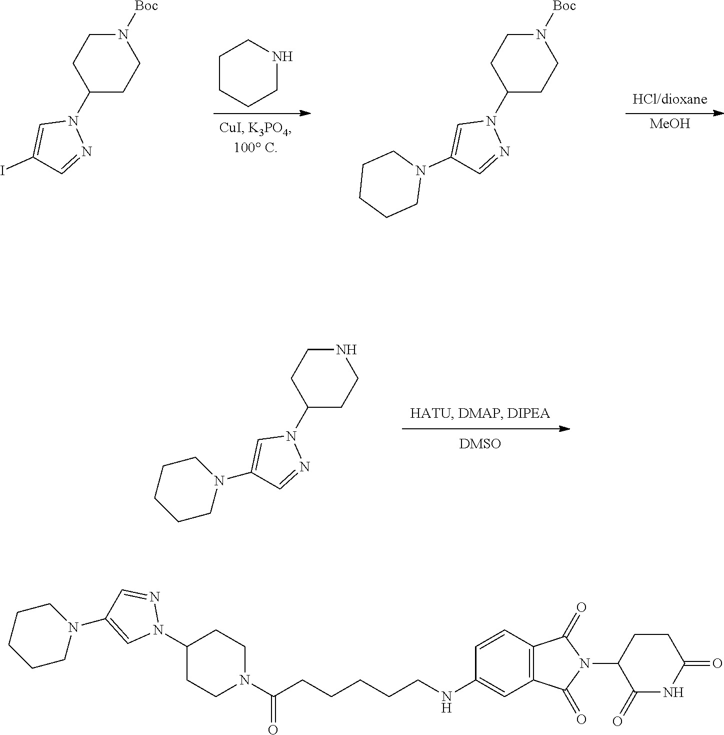 [SMILES: "MeOH" ; CC(C)(C)OC(=O)N1CCC(n2cc(I)cn2)CC1 ; C1CCNCC1 ; CC(C)(C)OC(=O)N1CCC(n2cc(N3CCCCC3)cn2)CC1 ; c1nn(C2CCNCC2)cc1N1CCCCC1 ; O=C1CCC(N2C(=O)c3ccc(NCCCCCC(=O)N4CCC(n5cc(N6CCCCC6)cn5)CC4)cc3C2=O)C(=O)N1]

GS-048 was synthesized following the similar procedures as described for GS-046 (3.8 mg) as yellow solid. MS (ESI) m/z=604.7 [M+H]⁺.

### Example 587. 2-(2,6-Dioxopiperidin-3-yl)-5-((6-(4-(4-morpholino-1H-pyrazol-1-yl)piperidin-1-yl)-6-oxohexyl)amino)isoindoline-1,3-dione (GS-047)

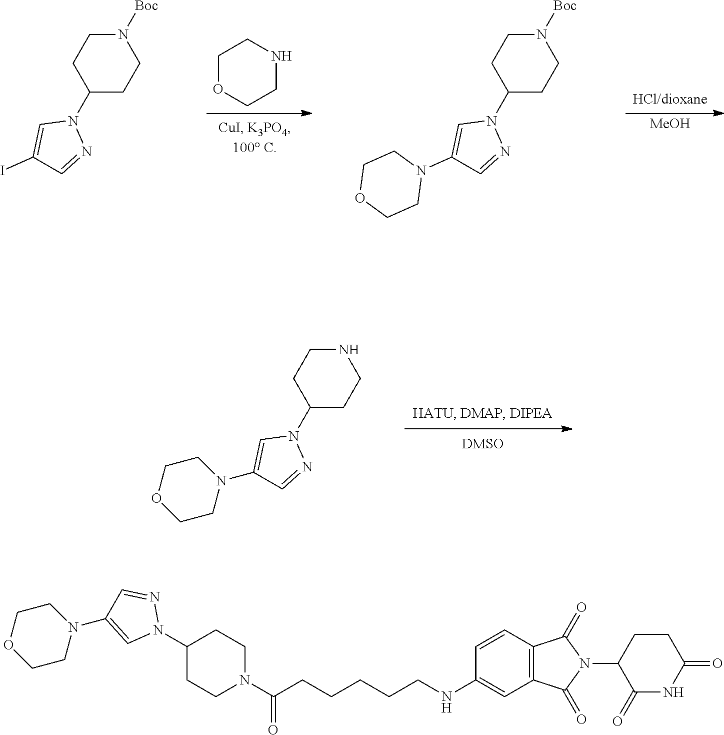 [SMILES: "MeOH" ; CC(C)(C)OC(=O)N1CCC(n2cc(I)cn2)CC1 ; C1COCCN1 ; CC(C)(C)OC(=O)N1CCC(n2cc(N3CCOCC3)cn2)CC1 ; c1nn(C2CCNCC2)cc1N1CCOCC1 ; O=C1CCC(N2C(=O)c3ccc(NCCCCCC(=O)N4CCC(n5cc(N6CCOCC6)cn5)CC4)cc3C2=O)C(=O)N1]

GS-047 was synthesized following the similar procedures as described for GS-046 (2.5 mg) as yellow solid. MS (ESI) m/z=606.6 [M+H]⁺.

### Example 588. 2-(2,6-Dioxopiperidin-3-yl)-5-((6-oxo-6-(4-(4-(8-(tetrahydro-2H-pyran-4-yl)quinoxalin-2-yl)-1H-pyrazol-1-yl)piperidin-1-yl)hexyl)amino)isoindoline-1,3-dione (GS-043)

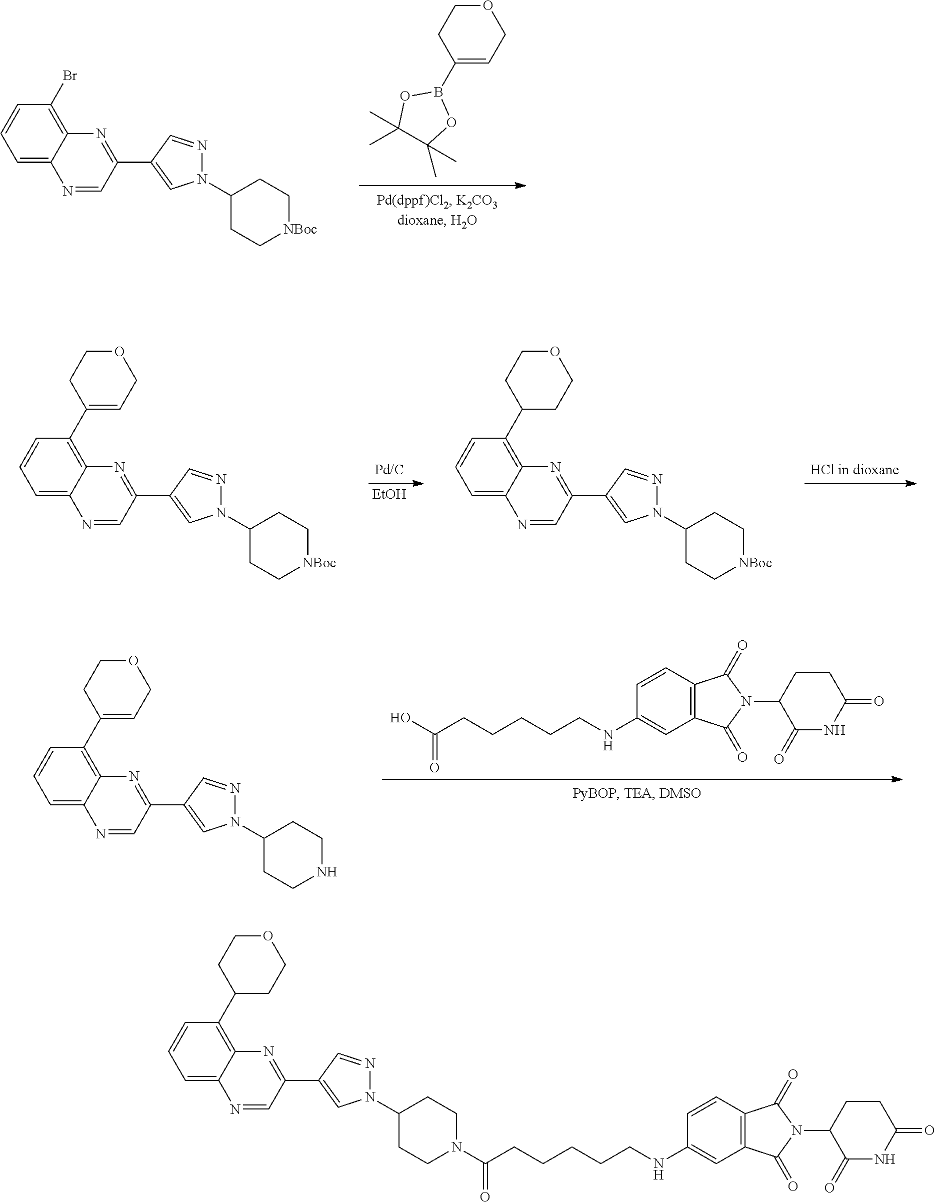

Step 1 to 2. Synthesis of tert-butyl 4-(4-(8-(tetrahydro-2H-pyran-4-yl)quinoxalin-2-yl)-1H-pyrazol-1-yl)piperidine-1-carboxylate

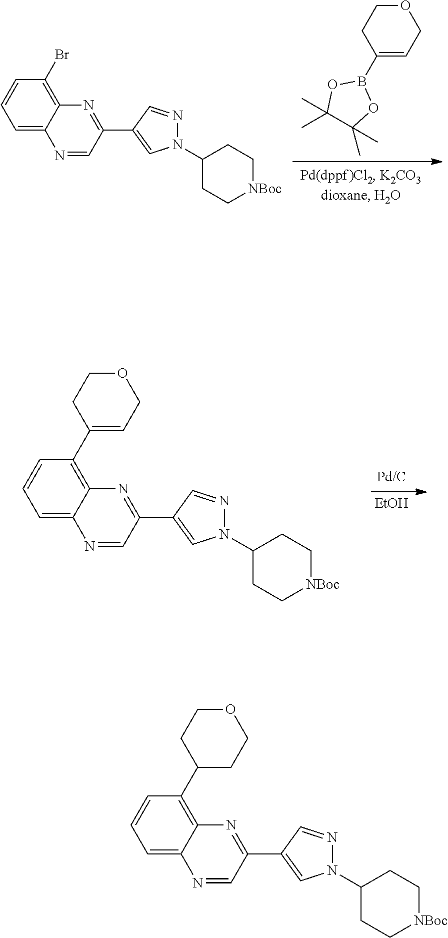

The title compound was synthesized following the similar procedures as described for steps 3 to 4 of GS-044 (50 mg). MS (ESI) m/z=464.5 [M+H]⁺.

Step 3 to 4. Synthesis of 2-(2,6-dioxopiperidin-3-yl)-5-((6-oxo-6-(4-(4-(8-(tetrahydro-2H-pyran-4-yl)quinoxalin-2-yl)-1H-pyrazol-1-yl)piperidin-1-yl)hexyl)amino)isoindoline-1,3-dione

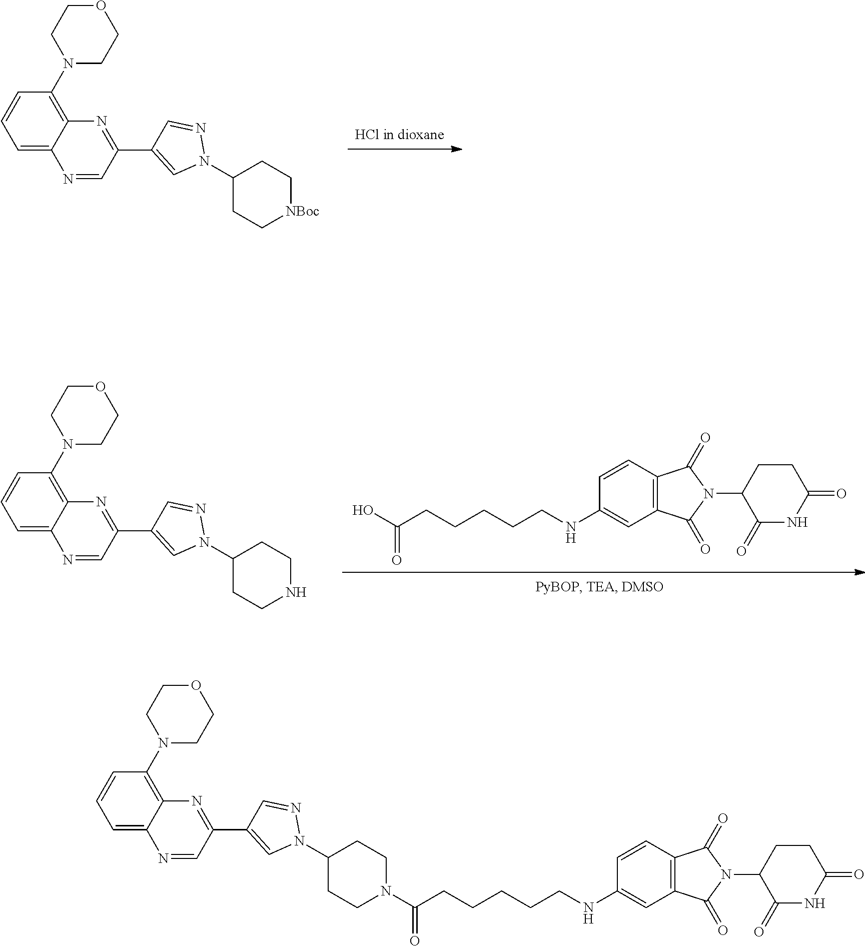 [SMILES: CC(C)(C)OC(=O)N1CCC(n2cc(-c3cnc4cccc(N5CCOCC5)c4n3)cn2)CC1 ; c1cc(N2CCOCC2)c2nc(-c3cnn(C4CCNCC4)c3)cnc2c1 ; O=C(O)CCCCCNc1ccc2c(c1)C(=O)N(C1CCC(=O)NC1=O)C2=O ; O=C1CCC(N2C(=O)c3ccc(NCCCCCC(=O)N4CCC(n5cc(-c6cnc7cccc(N8CCOCC8)c7n6)cn5)CC4)cc3C2=O)C(=O)N1]

GS-043 was synthesized following the similar procedures as described for GS-005 (33.3 mg). MS (ESI) m/z=733.9 [M+H]⁺.

### Example 589. 2-(2,6-Dioxopiperidin-3-yl)-5-((6-(4-(4-(8-(1-methylpiperidin-4-yl)quinoxalin-2-yl)-1H-pyrazol-1-yl)piperidin-1-yl)-6-oxohexyl)amino)isoindoline-1,3-dione (GS-045)

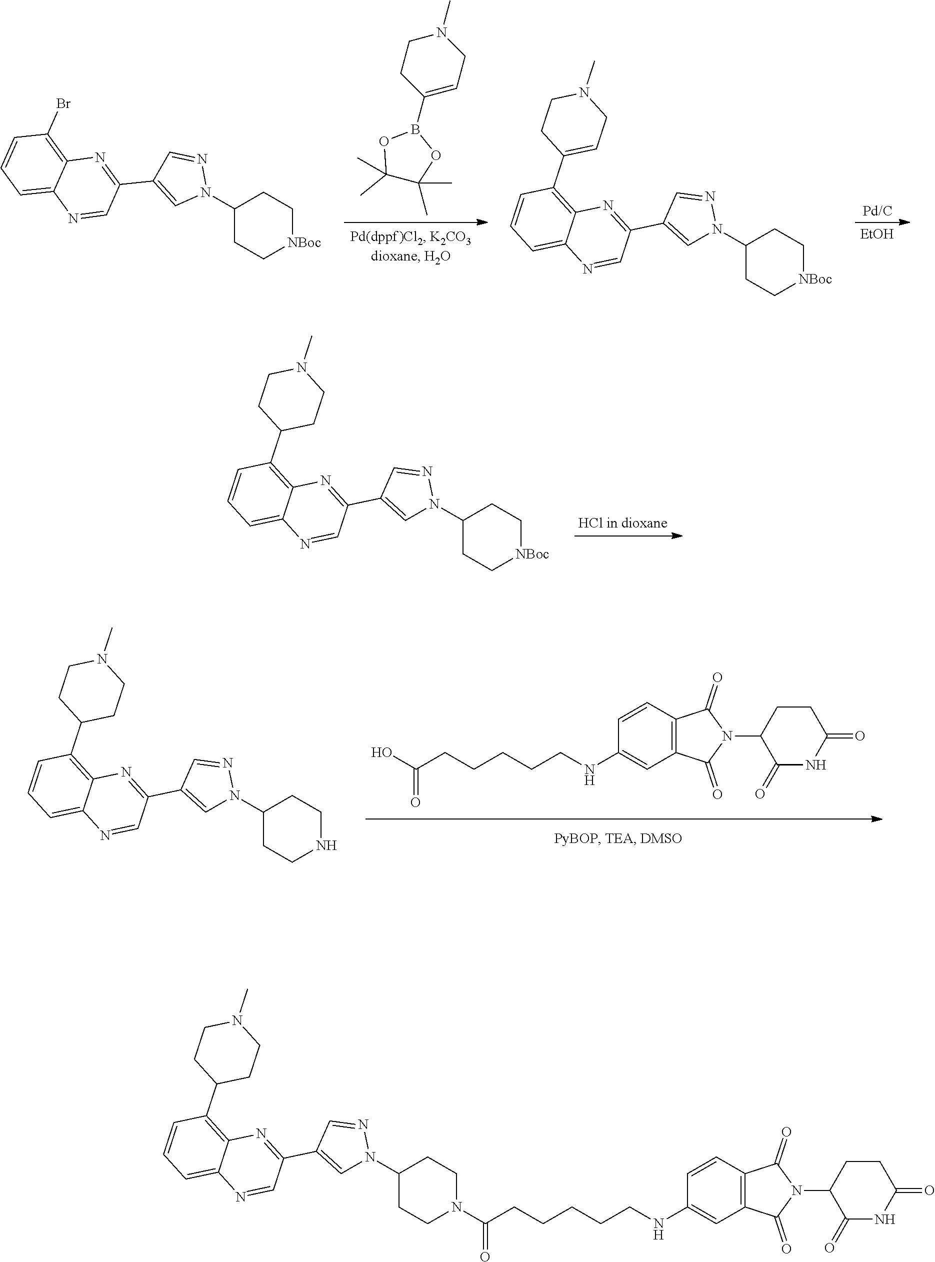

GS-045 was synthesized following the similar procedures as descried for GS-043 (115 mg). MS (ESI) m/z=746.8 [M+H]⁺.

### Example 590. 5-(((trans-3-(3-Cyclopropyl-4-(3-(piperazin-1-yl)pyridin-2-yl)-1H-pyrazol-1-yl(cyclobutyl)methyl)amino)-2-(2,6-dioxopiperidin-3-yl)isoindoline-1,3-dione (GS-604)

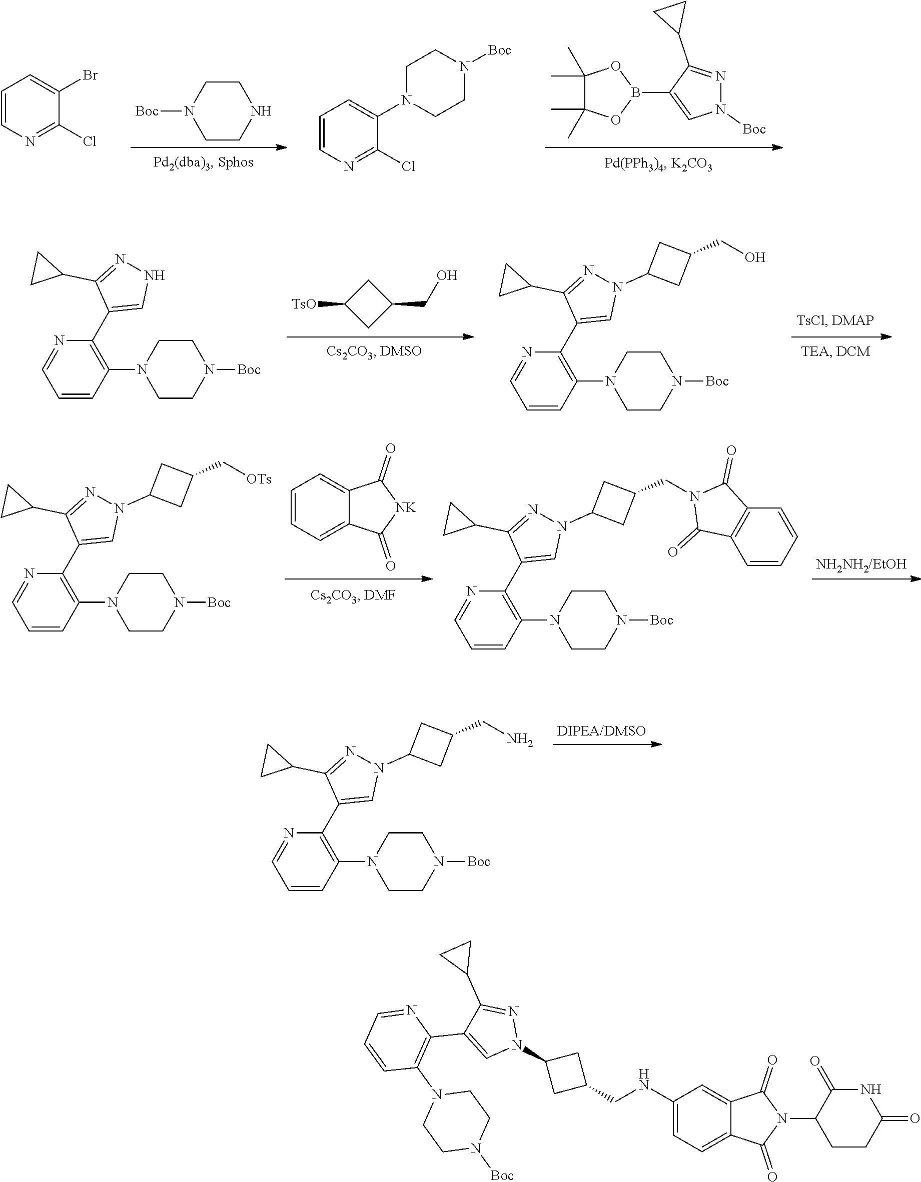

Step 1. Synthesis of tert-butyl 4-(2-chloropyridin-3-yl)piperazine-1-carboxylate

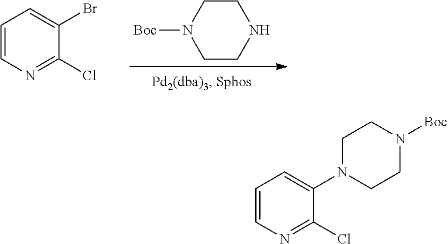 [SMILES: Clc1ncccc1Br ; CC(C)(C)OC(=O)N1CCNCC1 ; CC(C)(C)OC(=O)N1CCN(c2cccnc2Cl)CC1]

To a solution of 3-bromo-2-chloro-pyridine (2 g, 10.39 mmol) and tert-butyl piperazine-1-carboxylate (1.94 g, 10.39 mmol) in DMSO (100 mL) were added Cs₂CO₃ (10.13 g, 31.18 mmol), Pd₂(dba)₃ (950.94 mg, 1.04 mmol) and Sphos (852.21 mg, 2.08 mmol) under N₂, the reaction mixture was stirred at 100° C. for 3 h, then it was quenched with brine (100 mL), extracted with EtOAc (100 mL*2). The combined prganic layers were washed with brine (100 mL*2), dried over Na₂SO₄, filtered, and concentrated. The resulting residue was purified by silica gel chromatography (petroleum ether/EtOAc=15/1 to 10/1) to provide the title compound (1.0 g, 32.3% yield). MS (ESI) m/z=298.4 [M+H]⁺.

Step 2. Synthesis of tert-butyl 4-(2-(3-cyclopropyl-1H-pyrazol-4-yl)pyridin-3-yl)piperazine-1-carboxylate

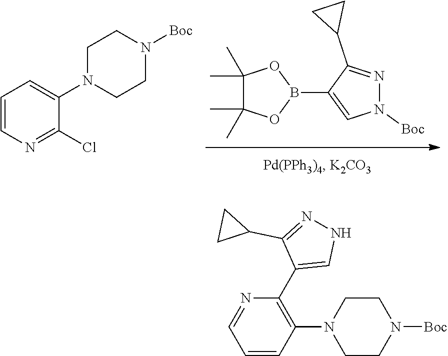 [SMILES: CC(C)(C)OC(=O)N1CCN(c2cccnc2Cl)CC1 ; CC(C)(C)OC(=O)n1cc(B2OC(C)(C)C(C)(C)O2)c(C2CC2)n1 ; CC(C)(C)OC(=O)N1CCN(c2cccnc2-c2c[nH]nc2C2CC2)CC1]

To a solution of tert-butyl 4-(2-chloro-3-pyridyl)piperazine-1-carboxylate (1.0 g, 3.36 mmol) and tert-butyl 3-cyclopropyl-4-(3,3,4,4-tetramethyl-1,2,5-bromadioxolan-1-yl)pyrazole-1-carboxylate (1.35 g, 3.36 mmol) in dioxane (20 mL) and water (10 mL) were added K₂CO₃ (1.39 g, 10.07 mmol) and Pd(PPh₃)₄ (387.87 mg, 335.82 μmol), the reaction mixture was stirred at 100° C. for 48 h, then it was concentrated and purified by silica gel chromatography (petroleum ether/EtOAc=1/1 to 0/1) to provide the title compound (250 mg, 20.2% yield). MS (ESI) m/z=370.7 [M+H]⁺.

Step 3. Synthesis of tert-butyl 4-(2-(3-cyclopropyl-1-(trans-3-(hydroxymethyl)cyclobutyl)-1H-pyrazol-4-yl)pyridin-3-yl)piperazine-1-carboxylate

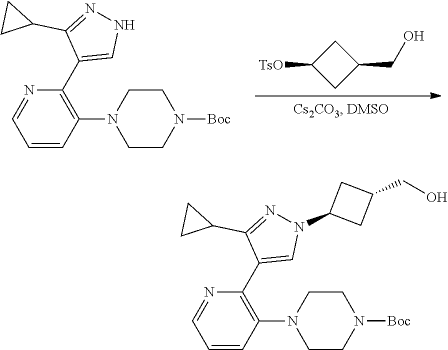 [SMILES: CC(C)(C)OC(=O)N1CCN(c2cccnc2-c2c[nH]nc2C2CC2)CC1 ; Cc1ccc(S(=O)(=O)O[C@H]2C[C@@H](CO)C2)cc1 ; CC(C)(C)OC(=O)N1CCN(c2cccnc2-c2cn([C@H]3C[C@H](CO)C3)nc2C2CC2)CC1]

To a solution of tert-butyl 4-(2-(3-cyclopropyl-1H-pyrazol-4-yl)pyridin-3-yl)piperazine-1-carboxylate (250 mg, 676.66 μmol) and cis-3-(hydroxymethyl)cyclobutyl 4-methylbenzenesulfonate (173.44 mg, 676.66 μmol) in DMSO (10 mL) was added Cs₂CO₃ (659.75 mg, 2.03 mmol), the reaction mixture was stirred at 100° C. for 16 h, then it was quenched with brine (20 mL), extracted with EtOAc (20 mL*3). The combined organic layers were washed with brine (20 ml*3) and concentrated. The resulting residue was purified by silica gel chromatography (petroleum ether/EtOAc=1/1 to 0/1) to provide the title compound (130 mg, 42.4% yield). MS (ESI) m/z=454.8 [M+H]⁺.

Step 4. Synthesis of tert-butyl 4-(2-(3-cyclopropyl-1-(trans-3-((tosyloxy)methyl)cyclobutyl)-1H-pyrazol-4-yl)pyridin-3-yl)piperazine-1-carboxylate

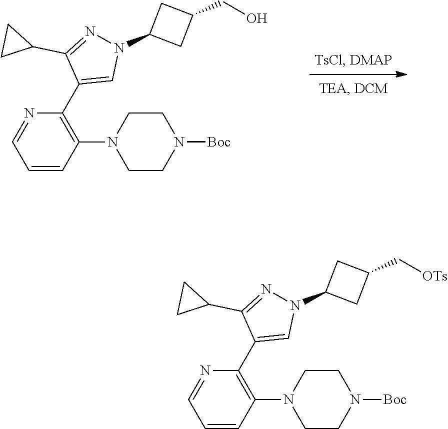 [SMILES: CC(C)(C)OC(=O)N1CCN(c2cccnc2-c2cn([C@H]3C[C@H](CO)C3)nc2C2CC2)CC1 ; Cc1ccc(S(=O)(=O)OC[C@H]2C[C@H](n3cc(-c4ncccc4N4CCN(C(=O)OC(C)(C)C)CC4)c(C4CC4)n3)C2)cc1]

To a solution of tert-butyl 4-(2-(3-cyclopropyl-1-(trans-3-(hydroxymethyl)cyclobutyl)-1H-pyrazol-4-yl)pyridin-3-yl)piperazine-1-carboxylate (130 mg, 286.61 μmol) in TEA (5 mL) and DCM (10 mL) were added DMAP (35.02 mg, 286.61 μmol) and 4-methylbenzenesulfonyl chloride (54.64 mg, 286.61 μmol). The resulting mixture was stirred at rt. for 2 h, then it was concentrated and purified by silica gel chromatography (petroleum ether/EtOAc=10/1 to 1/1) to provide the title compound (100 mg, 57.4% yield). MS (ESI) m/z=608.8 [M+H]⁺.

Step 5. Synthesis of tert-butyl 4-(2-(3-cyclopropyl-1-(trans-3-((1,3-dioxoisoindolin-2-yl)methyl)cyclobutyl)-1H-pyrazol-4-yl)pyridin-3-yl)piperazine-1-carboxylate

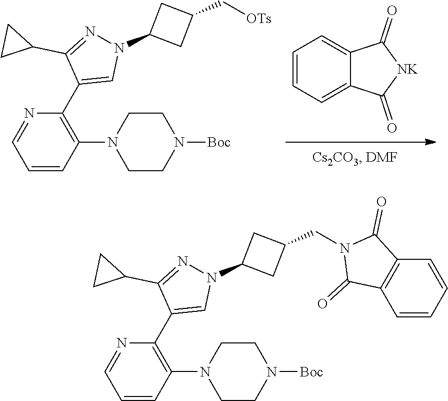

To a solution of tert-butyl 4-(2-(3-cyclopropyl-1-(trans-3-((tosyloxy)methyl)cyclobutyl)-1H-pyrazol-4-yl)pyridin-3-yl)piperazine-1-carboxylate (100 mg, 164.54 μmol) and (1,3-dioxoisoindolin-2-yl)potassium (30.48 mg, 164.54 μmol) in DMF (8 mL) was added Cs₂CO₃ (106.95 mg, 329.08 μmol). The reaction mixture was stirred at 65° C. for 2 h, then it was quenched with brine (20 mL) and extracted with EtOAc (20 mL*3). The combined organic layers were washed with brine (20 mL*3) and concentrated to provide crude product (95 mg, 99.1% yield) which was used directly in the next step without further purification. MS (ESI) m/z=583.8 [M+H]⁺.

Step 6. Synthesis of tert-butyl 4-(2-(1-(trans-3-(aminomethyl)cyclobutyl)-3-cyclopropyl-1H-pyrazol-4-yl)pyridin-3-yl)piperazine-1-carboxylate

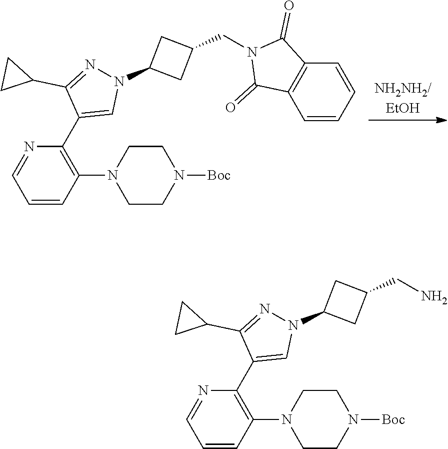 [SMILES: CC(C)(C)OC(=O)N1CCN(c2cccnc2-c2cn([C@H]3C[C@H](CN4C(=O)c5ccccc5C4=O)C3)nc2C2CC2)CC1 ; CC(C)(C)OC(=O)N1CCN(c2cccnc2-c2cn([C@H]3C[C@H](CN)C3)nc2C2CC2)CC1]

To a solution of tert-butyl 4-(2-(3-cyclopropyl-1-(trans-3-((1,3-dioxoisoindolin-2-yl)methyl)cyclobutyl)-1H-pyrazol-4-yl)pyridin-3-yl)piperazine-1-carboxylate (95 mg, 163.04 μmol) in EtOH (5 mL) was added NH₂NH₂ (80%, 2 mL). The reaction mixture was stirred at 50° C. for 1 h, then it was concentrated and purified by prep-HPLC to provide the title compound (70 mg, 75.8% yield). MS (ESI) m/z=483.6 [M+H]⁺.

Step 7. Synthesis of tert-butyl 4-(2-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)pyridin-3-yl)piperazine-1-carboxylate

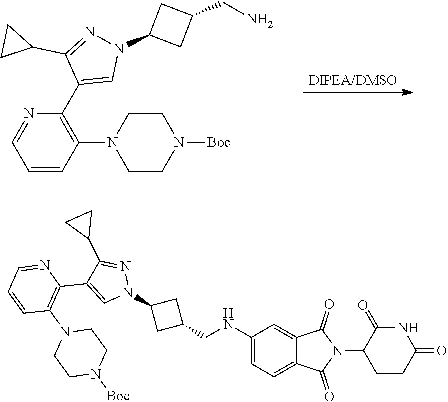 [SMILES: CC(C)(C)OC(=O)N1CCN(c2cccnc2-c2cn([C@H]3C[C@H](CN)C3)nc2C2CC2)CC1 ; CC(C)(C)OC(=O)N1CCN(c2cccnc2-c2cn([C@H]3C[C@H](CNc4ccc5c(c4)C(=O)N(C4CCC(=O)NC4=O)C5=O)C3)nc2C2CC2)CC1]

To a solution of tert-butyl 4-(2-(1-(trans-3-(aminomethyl)cyclobutyl)-3-cyclopropyl-1H-pyrazol-4-yl)pyridin-3-yl)piperazine-1-carboxylate (70 mg, 102.84 μmol) and 2-(2,6-dioxo-3-piperidyl)-5-fluoro-isoindoline-1,3-dione (56.82 mg, 205.69 μmol) in DMSO (8 mL) was added DPEA (2 mL). The reaction mixture was stirred at 135° C. for 2 h, then it was cooled to rt and quenched with bine (20 mL), extracted with EtOAc (20 mL*3). The combined organic layers were washed with brine (20 mL*3), dried over Na₂SO₄, filtrated and concentrated. The resulting residue was purified by prep-TLC (DCM/MeOH=15/1) to provide the title compound (20 mg, 27.4% yield). MS (ESI) m/z=709.9 [M+H]⁺.

Step 8. Synthesis of 5-(((trans-3-(3-cyclopropyl-4-(3-(piperazin-1-yl)pyridin-2-yl)-1H-pyrazol-1-yl)cyclobutyl)methyl)amino)-2-(2,6-dioxopiperidin-3-yl)isoindoline-1,3-dione

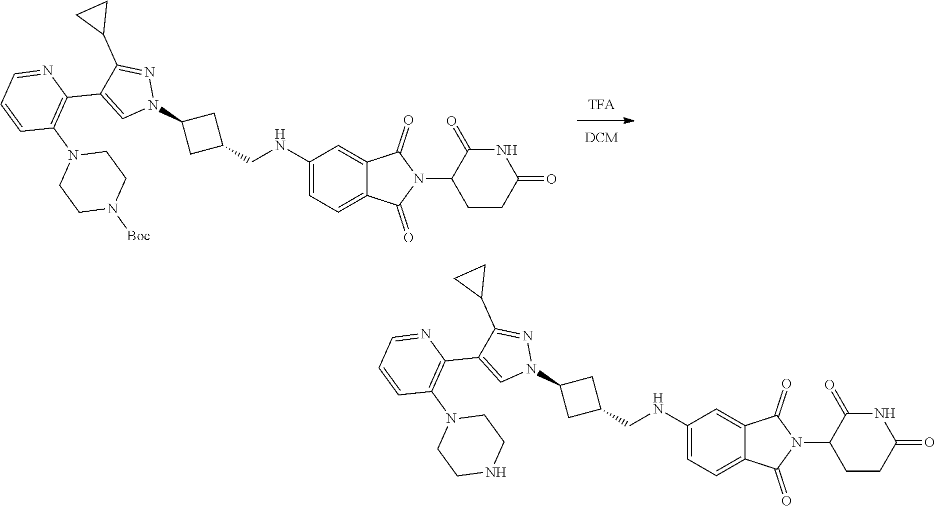 [SMILES: CC(C)(C)OC(=O)N1CCN(c2cccnc2-c2cn([C@H]3C[C@H](CNc4ccc5c(c4)C(=O)N(C4CCC(=O)NC4=O)C5=O)C3)nc2C2CC2)CC1 ; O=C1CCC(N2C(=O)c3ccc(NC[C@H]4C[C@H](n5cc(-c6ncccc6N6CCNCC6)c(C6CC6)n5)C4)cc3C2=O)C(=O)N1]

To a solution of tert-butyl 4-(2-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)pyridin-3-yl)piperazine-1-carboxylate (20 mg, 28.22 μmol) in DCM (4 mL) was added TFA (1 mL), the reaction mixture was stirred at rt for 0.5 h, then it was concentrated and purified by prep-TLC (DCM:MeOH=8:1) to provide the title compound (7.5 mg, 43.7% yield). MS (ESI) m/z=609.7 [M+H]⁺.

### Example 591. 5-(((trans-3-(4-(6-(Azetidin-3-yl)pyridin-2-yl)-3-cyclopropyl-1H-pyrazol-1-yl)cyclobutyl)methyl)amino)-2-(2,6-dioxopiperidin-3-yl)isoindoline-1,3-dione (GS-605)

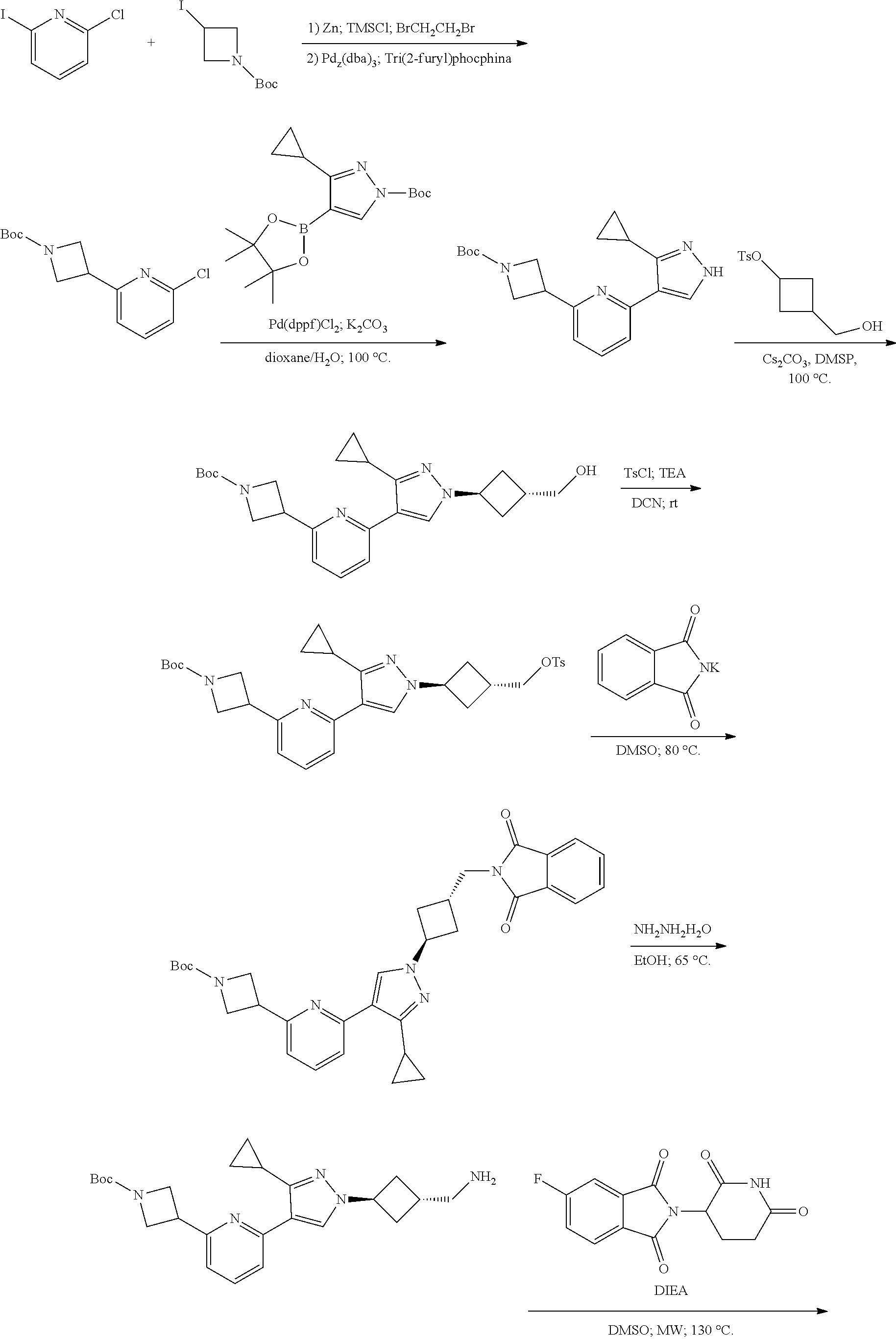

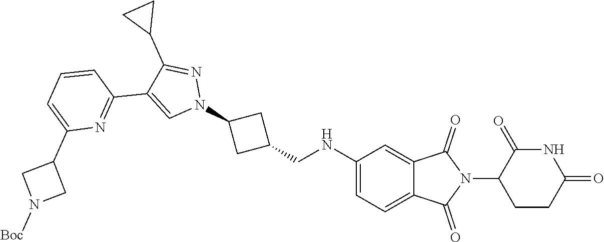 [SMILES: CC(C)(C)OC(=O)N1CC(c2cccc(-c3cn([C@H]4C[C@H](CNc5ccc6c(c5)C(=O)N(C5CCC(=O)NC5=O)C6=O)C4)nc3C3CC3)n2)C1]

Step 1. Synthesis of tert-butyl 3-(6-chloropyridin-2-yl)azetidine-1-carboxylate

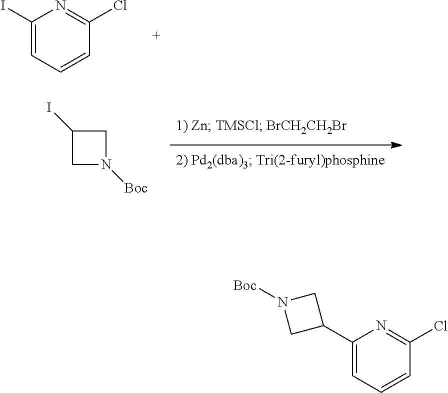

To a solution of tert-butyl 3-iodoazetidine-1-carboxylate (10.6 g, 37.44 mmol) in THF (50 mL) was added 1,2-Dibromoethane (234.46 mg, 1.25 mmol). After being stirred at 80° C. for 10 min, the reaction mixture was cooled and TMSCl (135.59 mg, 1.25 mmol) was added, stirred at rt for 45 min. Then a solution of tert-butyl 3-iodoazetidine-1-carboxylate (10.6 g, 37.44 mmol) in THF was added and stirred at 80° C. for 1 h. The reaction was cooled and Pd₂(dba)₃ (1.14 g, 1.25 mmol) and tri(2-furyl)phosphine (579.52 mg, 2.50 mmol) were added. Then the mixture was stirred at 60° C. for 12 h, concentrated and the resulting residue was purified by Flash column chromatography to provide the title compound (3.0 g, 89.4% yield) as a light-yellow oil. MS (ESI) m/z=269.3 [M+H]⁺.

Step 2. Synthesis of tert-butyl 3-(6-(3-cyclopropyl-1H-pyrazol-4-yl)pyridin-2-yl)azetidine-1-carboxylate

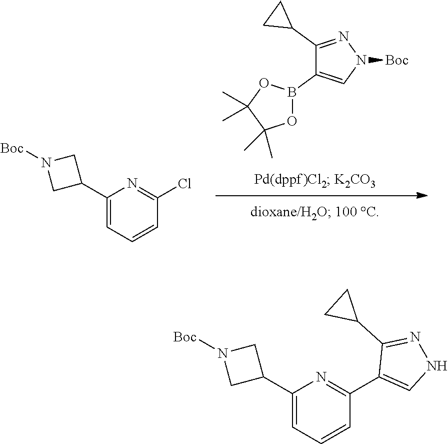

Under nitrogen atmosphere, a mixture of tert-butyl 3-(6-chloro-2-pyridyl)azetidine-1-carboxylate (300 mg, 1.12 mmol), tert-butyl 3-cyclopropyl-4-(4,4,5,5-tetramethyl-1,3,2-dioxaborolan-2-yl)pyrazole-1-carboxylate (485.02 mg, 1.45 mmol), Pd(dppf)Cl₂ (81.60 mg, 111.63 μmol) and K₂CO₃ (462.16 mg, 3.34 mmol) in dioxone (8 mL) and H₂O (2 mL) was stirred at 100° C. for 15 h, then it was diluted with EtOAc, washed with H₂O and brine, concentrated and the resulting residue was purified by flash column chromatography to provide the title compound (90 mg, 23.7% yield) as a white solid. MS (ESI) m/z=341.7 [M+H]⁺.

Step 3. Synthesis of tert-butyl 3-(6-(3-cyclopropyl-1-(trans-3-(hydroxymethyl)cyclobutyl)-1H-pyrazol-4-yl)pyridin-2-yl)azetidine-1-carboxylate

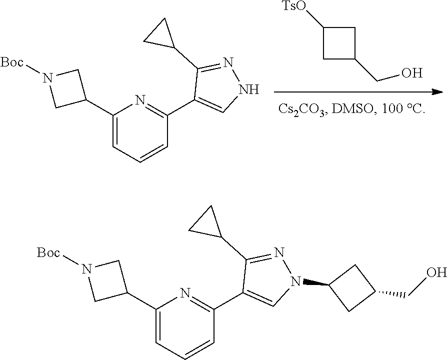 [SMILES: CC(C)(C)OC(=O)N1CC(c2cccc(-c3c[nH]nc3C3CC3)n2)C1 ; Cc1ccc(S(=O)(=O)OC2CC(CO)C2)cc1 ; CC(C)(C)OC(=O)N1CC(c2cccc(-c3cn([C@H]4C[C@H](CO)C4)nc3C3CC3)n2)C1]

To a solution of tert-butyl 3-(6-(3-cyclopropyl-1H-pyrazol-4-yl)pyridin-2-yl)azetidine-1-carboxylate (90 mg, 264.38 μmol) and 3-(hydroxymethyl)cyclobutyl 4-methylbenzenesulfonate (88.09 mg, 343.69 μmol) in DMSO (5 mL) was added Cs₂CO₃ (258.42 mg, 793.14 μmol), then it was stirred at 95° C. for 1 h. The reaction mixture was diluted with EtOAc (20 mL), washed with H₂O and brine, concentrated and the resulting residue was purified Flash column chromatography to provide the title compound (60 mg, 53.5% yield) as a white solid. MS (ESI) m/z=425.7 [M+H]⁺.

Step 4. Synthesis of tert-butyl 3-(6-(3-cyclopropyl-1-(trans-3-((tosyloxy)methyl)cyclobutyl)-1H-pyrazol-4-yl)pyridin-2-yl)azetidine-1-carboxylate

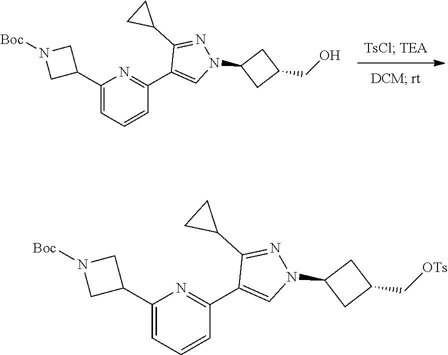 [SMILES: CC(C)(C)OC(=O)N1CC(c2cccc(-c3cn([C@H]4C[C@H](CO)C4)nc3C3CC3)n2)C1 ; Cc1ccc(S(=O)(=O)OC[C@H]2C[C@H](n3cc(-c4cccc(C5CN(C(=O)OC(C)(C)C)C5)n4)c(C4CC4)n3)C2)cc1]

To a solution of tert-butyl 3-(6-(3-cyclopropyl-1-(trans-3-(hydroxymethyl)cyclobutyl)-1H-pyrazol-4-yl)pyridin-2-yl)azetidine-1-carboxylate (60 mg, 141.33 μmol) and TsCl (14.95 mg, 212 μmol) in DCM (5 mL) were added DMAP (1.73 mg, 14.13 μmol) and TEA (286.03 mg, 2.83 mmol), then it was stirred at rt for 3 h. The reaction mixture was diluted with DCM (20 mL), washed with H₂O and brine, concentrated and The resulting residue was purified by prep-TLC to provide the title compound (60 mg, 73.4% yield) as a white solid. MS (ESI) m/z=579.7 [M+H]P.

Step 5. Synthesis of tert-butyl 3-(6-(3-cyclopropyl-1-(trans-3-((1,3-dioxoisoindolin-2-yl)methyl)cyclobutyl)-1H-pyrazol-4-yl)pyridin-2-yl)azetidine-1-carboxylate

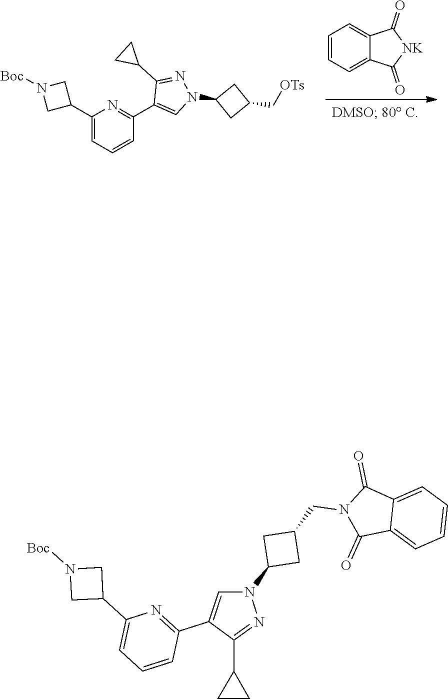

To a solution of tert-butyl 3-(6-(3-cyclopropyl-1-(trans-3-((tosyloxy)methyl)cyclobutyl)-1H-pyrazol-4-yl)pyridin-2-yl)azetidine-1-carboxylate (60 mg, 103.68 μmol) and (1,3-dioxoisoindolin-2-yl)potassium (28.80 mg, 155.52 μmol) in DMSO (5 mL) was added Cs₂CO₃ (67.56 mg, 207.35 μmol).

The reaction mixture was stirred at 80° C. for 30 min, then it was diluted with EtOAc (20 mL) and washed with H₂O and brine, concentrated to provide the title compound (42 mg, 73.2% yield) as a white solid. MS (ESI) m/z=554.8 [M+H]⁺.

Step 6. Synthesis of tert-butyl 3-(6-(1-(trans-3-(aminomethyl)cyclobutyl)-3-cyclopropyl-1H-pyrazol-4-yl)pyridin-2-yl)azetidine-1-carboxylate

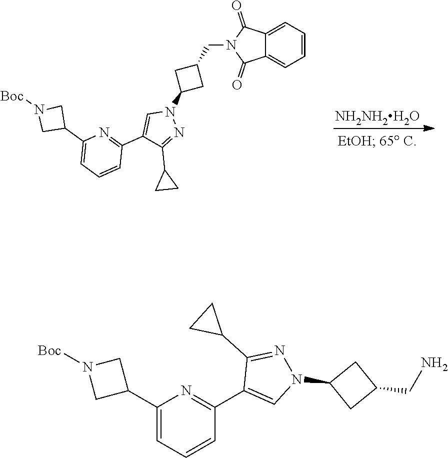 [SMILES: CC(C)(C)OC(=O)N1CC(c2cccc(-c3cn([C@H]4C[C@H](CN5C(=O)c6ccccc6C5=O)C4)nc3C3CC3)n2)C1 ; "NH2NH2·H2O" ; CC(C)(C)OC(=O)N1CC(c2cccc(-c3cn([C@H]4C[C@H](CN)C4)nc3C3CC3)n2)C1]

To a solution of tert-butyl 3-(6-(3-cyclopropyl-1-(trans-3-((1,3-dioxoisoindolin-2-yl)methyl)cyclobutyl)-1H-pyrazol-4-yl)pyridin-2-yl)azetidine-1-carboxylate (45 mg, 81.28 μmol) in EtOH (2 mL) was added NH₂NH₂·H₂O (130.23 mg, 4.06 mmol). The reaction mixture was stirred at 90° C. for 30 min, then it was diluted with EtOAc, washed with H₂O and brine, concentrated and The resulting residue was purified by flash column chromatography to provide the title compound (26 mg, 59.5% yield) as a white solid. MS (ESI) m/z=381.3 [M+H]⁺.

Step 7. Synthesis of tert-butyl 3-(6-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)pyridin-2-yl)azetidine-1-carboxylate

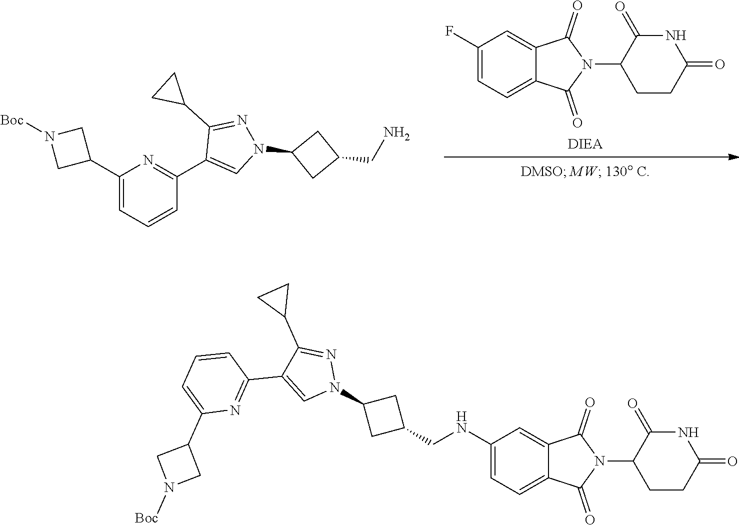 [SMILES: CC(C)(C)OC(=O)N1CC(c2cccc(-c3cn([C@H]4C[C@H](CN)C4)nc3C3CC3)n2)C1 ; O=C1CCC(N2C(=O)c3ccc(F)cc3C2=O)C(=O)N1 ; CC(C)(C)OC(=O)N1CC(c2cccc(-c3cn([C@H]4C[C@H](CNc5ccc6c(c5)C(=O)N(C5CCC(=O)NC5=O)C6=O)C4)nc3C3CC3)n2)C1]

To a solution of tert-butyl 3-(6-(1-(trans-3-(aminomethyl)cyclobutyl)-3-cyclopropyl-1H-pyrazol-4-yl)pyridin-2-yl)azetidine-1-carboxylate (26 mg, 61.39 μmol) in DMSO (0.5 mL) were added 2-(2,6-dioxo-3-piperidyl)-5-fluoro-isoindoline-1,3-dione (25.43 mg, 92.08 μmol) and DIEA (78.57 mg, 613.86 μmol), then it was stirred at 130° C. under microwave irradiation reactor for 2 h. The reaction mixture was purified by prep-HPLC to provide the title compound (15 mg, 39.1% yield) as a yellow solid. MS (ESI) m/z=680.8 [M+H]⁺.

Step 8. Synthesis of 5-(((trans-3-(4-(6-(azetidin-3-yl)pyridin-2-yl)-3-cyclopropyl-1H-pyrazol-1-yl)cyclobutyl)methyl)amino)-2-(2,6-dioxopiperidin-3-yl)isoindoline-1,3-dione

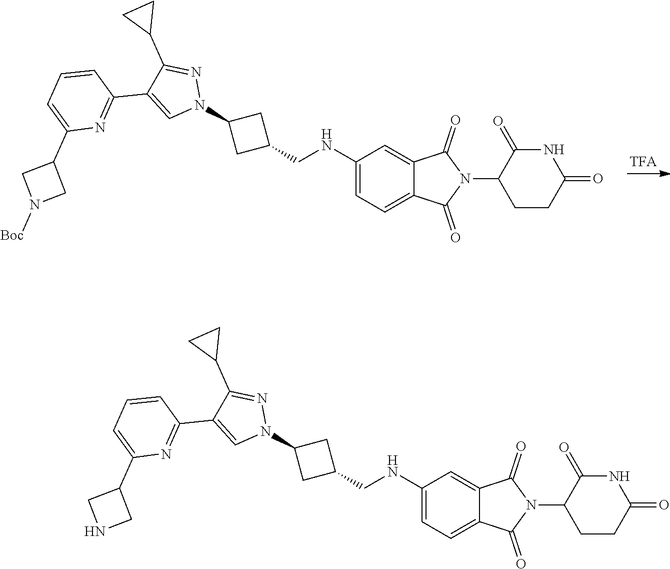 [SMILES: CC(C)(C)OC(=O)N1CC(c2cccc(-c3cn([C@H]4C[C@H](CNc5ccc6c(c5)C(=O)N(C5CCC(=O)NC5=O)C6=O)C4)nc3C3CC3)n2)C1 ; O=C1CCC(N2C(=O)c3ccc(NC[C@H]4C[C@H](n5cc(-c6cccc(C7CNC7)n6)c(C6CC6)n5)C4)cc3C2=O)C(=O)N1]

To a solution of tert-butyl 3-(6-(3-cyclopropyl-1-(trans-3-(((2-(2,6-dioxopiperidin-3-yl)-1,3-dioxoisoindolin-5-yl)amino)methyl)cyclobutyl)-1H-pyrazol-4-yl)pyridin-2-yl)azetidine-1-carboxylate (12 mg, 17.25 μmol) in DCM (2 mL) was added TFA (19.66 mg, 172.46 μmol) at 0° C., then it was stirred at the temperature for 1 h. The reaction mixture was concentrated to provide the title compound (3.5 mg, 34.1% yield) as a yellow solid. MS (ESI) m/z=580.7 [M+H]⁺.

### Example 592. 5-(((trans-3-(3-Cyclopropyl-4-(4-(dimethylamino)pyridin-2-yl)-1H-pyrazol-1-yl)cyclobutyl)methyl)amino)-2-(2,6-dioxopiperidin-3-yl)isoindoline-1,3-dione (GS-606)

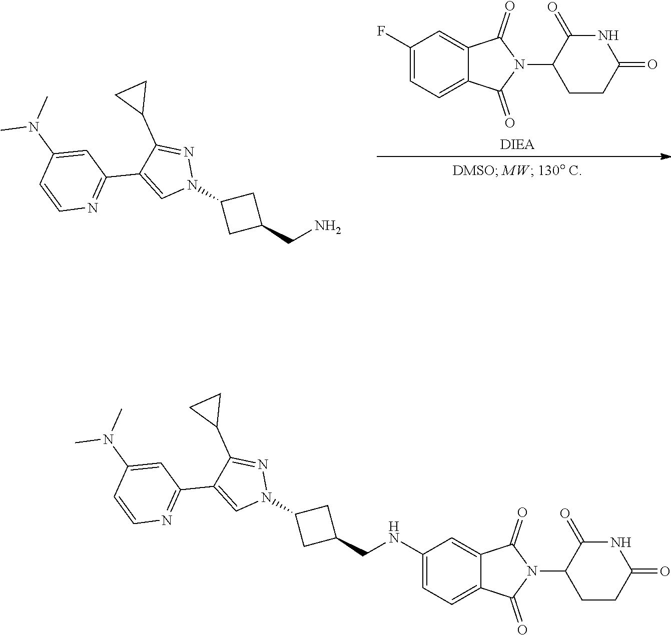 [SMILES: CN(C)c1ccnc(-c2cn([C@H]3C[C@H](CN)C3)nc2C2CC2)c1 ; O=C1CCC(N2C(=O)c3ccc(F)cc3C2=O)C(=O)N1 ; CN(C)c1ccnc(-c2cn([C@H]3C[C@H](CNc4ccc5c(c4)C(=O)N(C4CCC(=O)NC4=O)C5=O)C3)nc2C2CC2)c1]

GS-606 was synthesized following the similar procedure for preparing GS-604 (4.7 mg, yield: 27.6%). MS (ESI) m/z=568.7 [M+H]⁺.

### Example 593. 5-(((trans-3-(3-Cyclopropyl-4-(4-(piperazin-1-yl)pyridin-2-yl)-1H-pyrazol-1-yl)cyclobutyl)methyl)amino)-2-(2,6-dioxopiperidin-3-yl)isoindoline-1,3-dione (GS-607)

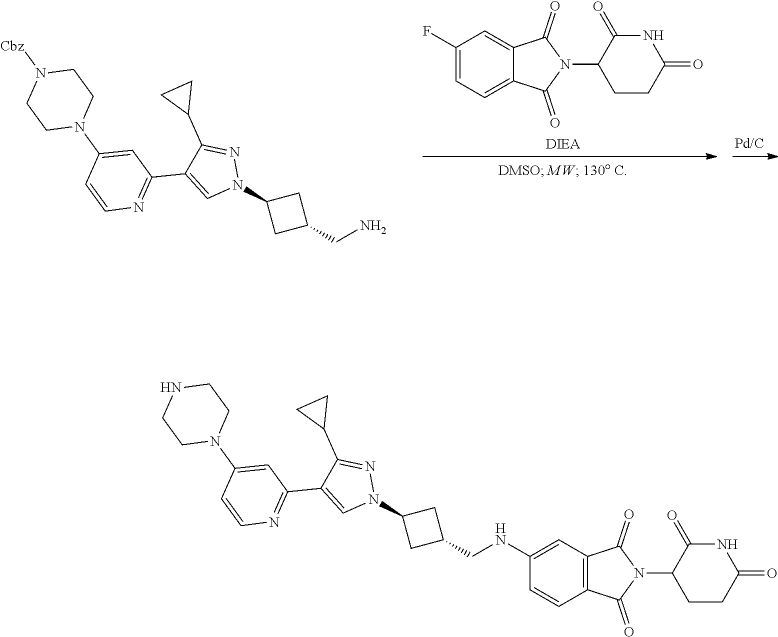 [SMILES: NC[C@H]1C[C@H](n2cc(-c3cc(N4CCN(C(=O)OCc5ccccc5)CC4)ccn3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cc(N7CCNCC7)ccn6)c(C6CC6)n5)C4)cc3C2=O)C(=O)N1]

GS-607 was synthesized following the similar procedure for preparing GS-294 (1.2 mg, yield: 3.2%). MS (ESI) m/z=609.7 [M+H]⁺.

### Example 594. 5-(((trans-3-(3-Cyclopropyl-4-(4-(4-methylpiperazin-1-yl)pyridin-2-yl)-1H-pyrazol-1-yl)cyclobutyl)methyl)amino)-2-(2,6-dioxopiperidin-3-yl)isoindoline-1,3-dione (GS-608)

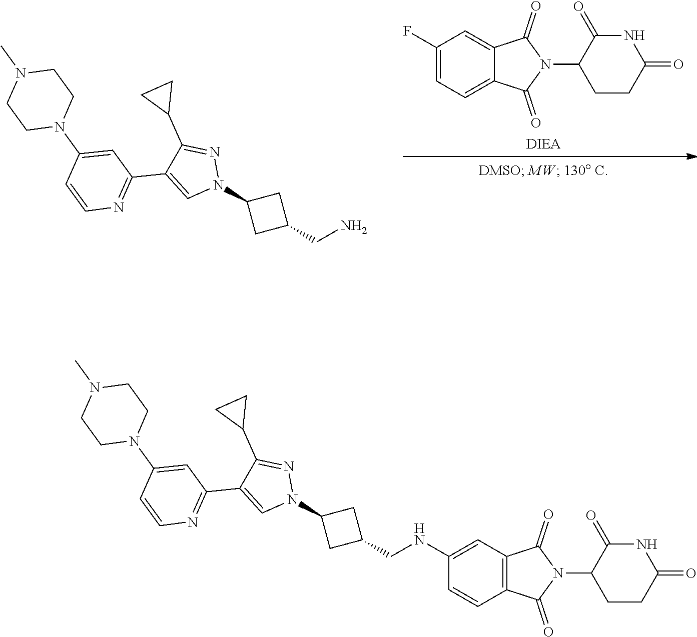 [SMILES: CN1CCN(c2ccnc(-c3cn([C@H]4C[C@H](CN)C4)nc3C3CC3)c2)CC1 ; O=C1CCC(N2C(=O)c3ccc(F)cc3C2=O)C(=O)N1 ; CN1CCN(c2ccnc(-c3cn([C@H]4C[C@H](CNc5ccc6c(c5)C(=O)N(C5CCC(=O)NC5=O)C6=O)C4)nc3C3CC3)c2)CC1]

GS-608 was synthesized following the similar procedure for preparing GS-604 (3 mg, yield: 7%). MS (ESI) m/z=623.8 [M+H]⁺.

### Example 595. 5-(((trans-3-(4-(5-Chloro-6-methylpyridin-2-yl)-3-cyclopropyl-1H-pyrazol-1-yl)cyclobutyl)methyl)amino)-2-(2,6-dioxopiperidin-3-yl)isoindoline-1,3-dione (GS-609)

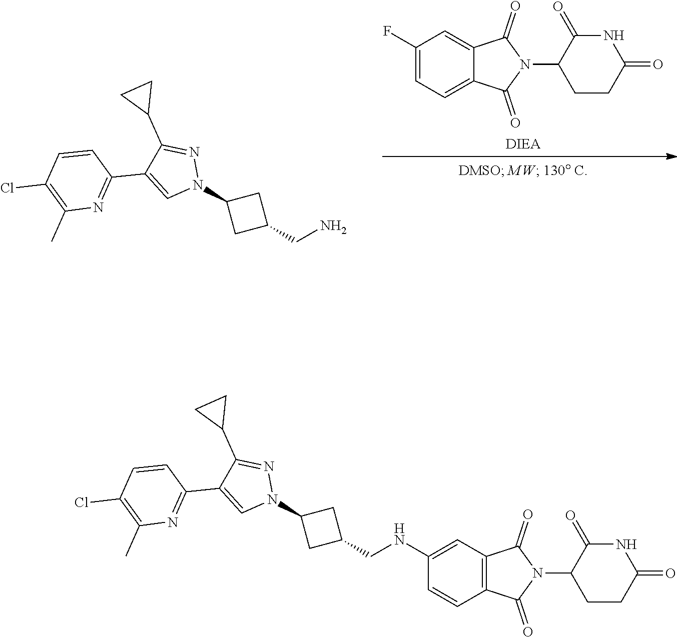 [SMILES: Cc1nc(-c2cn([C@H]3C[C@H](CN)C3)nc2C2CC2)ccc1Cl ; O=C1CCC(N2C(=O)c3ccc(F)cc3C2=O)C(=O)N1 ; Cc1nc(-c2cn([C@H]3C[C@H](CNc4ccc5c(c4)C(=O)N(C4CCC(=O)NC4=O)C5=O)C3)nc2C2CC2)ccc1Cl]

GS-609 was synthesized following the similar procedure for preparing GS-604 (20.8 mg, yield: 32.8%). MS (ESI) m/z=573.6 [M+H]⁺.

### Example 596. 5-(((trans-3-(4-(5-Chloro-6-methylpyridin-2-yl)-3-cyclopropyl-1H-pyrazol-1-yl)cyclobutyl)methyl)amino)-2-(2,6-dioxopiperidin-3-yl)isoindoline-1,3-dione (GS-610)

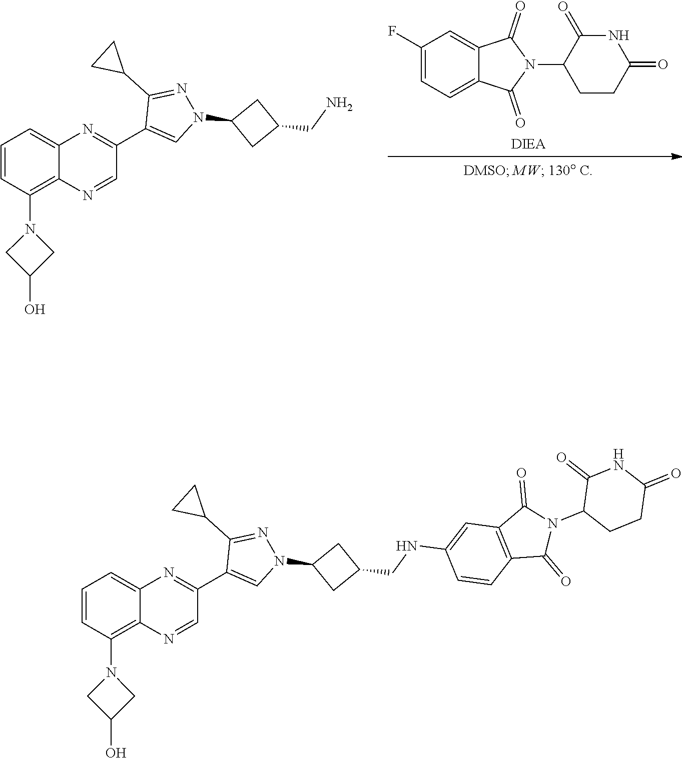 [SMILES: NC[C@H]1C[C@H](n2cc(-c3cnc4c(N5CC(O)C5)cccc4n3)c(C3CC3)n2)C1 ; O=C1CCC(N2C(=O)c3ccc(F)cc3C2=O)C(=O)N1 ; O=C1CCC(N2C(=O)c3ccc(NC[C@H]4C[C@H](n5cc(-c6cnc7c(N8CC(O)C8)cccc7n6)c(C6CC6)n5)C4)cc3C2=O)C(=O)N1]

GS-610 was synthesized following the similar procedure for preparing GS-604 (100 mg, yield: 36.5%). MS (ESI) m/z=647.7 [M+H]⁺.

### Example 597. 5-(((trans-3-(3-Cyclopropyl-4-(1-methyl-1H-pyrazolo[4,3-c]pyridin-4-yl)-1H-pyrazol-1-yl)cyclobutyl)methyl)amino)-2-(2,6-dioxopiperidin-3-yl)isoindoline-1,3-dione (GS-611)

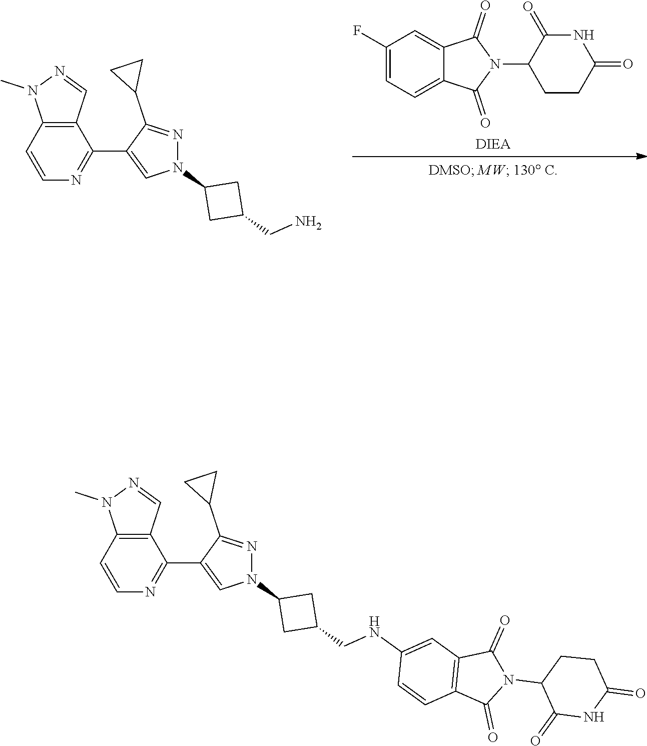 [SMILES: Cn1ncc2c(-c3cn([C@H]4C[C@H](CN)C4)nc3C3CC3)nccc21 ; O=C1CCC(N2C(=O)c3ccc(F)cc3C2=O)C(=O)N1 ; Cn1ncc2c(-c3cn([C@H]4C[C@H](CNc5ccc6c(c5)C(=O)N(C5CCC(=O)NC5=O)C6=O)C4)nc3C3CC3)nccc21]

GS-611 was synthesized following the similar procedure for preparing GS-613 (35 mg, yield: 19.5%). MS (ESI) m/z=579.7 [M+H]⁺.

### Example 598. 5-(((trans-3-(3-Cyclopropyl-4-(1-isopropyl-1H-pyrazolo[4,3-c]pyridin-4-yl)-1H-pyrazol-1-yl)cyclobutyl)methyl)amino)-2-(2,6-dioxopiperidin-3-yl)isoindoline-1,3-dione (GS-612)

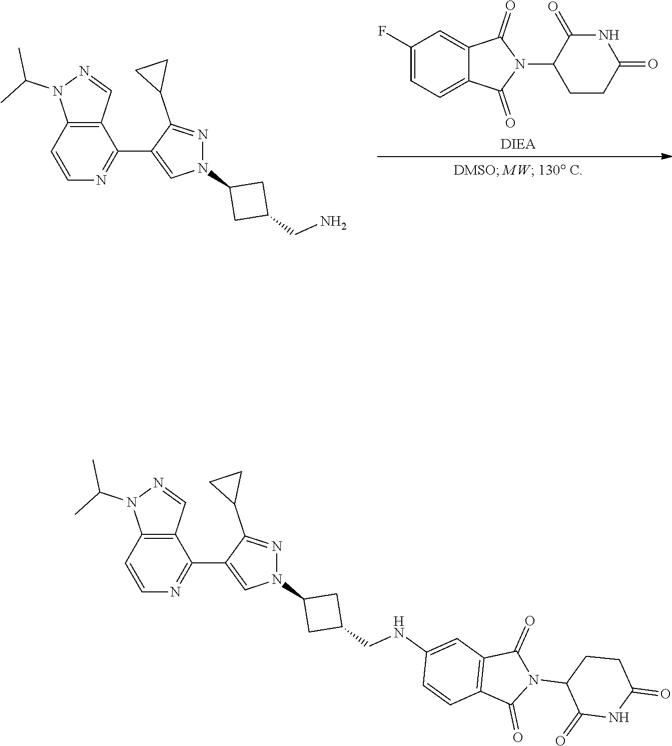 [SMILES: CC(C)n1ncc2c(-c3cn([C@H]4C[C@H](CN)C4)nc3C3CC3)nccc21 ; O=C1CCC(N2C(=O)c3ccc(F)cc3C2=O)C(=O)N1 ; CC(C)n1ncc2c(-c3cn([C@H]4C[C@H](CNc5ccc6c(c5)C(=O)N(C5CCC(=O)NC5=O)C6=O)C4)nc3C3CC3)nccc21]

GS-612 was synthesized following the similar procedure for preparing GS-613 (66 mg, yield: 25.4%). MS (ESI) m/z=607.7 [M+H]⁺.

### Example 599. 5-(((trans-3-(3-Cyclopropyl-4-(1-methyl-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-613)

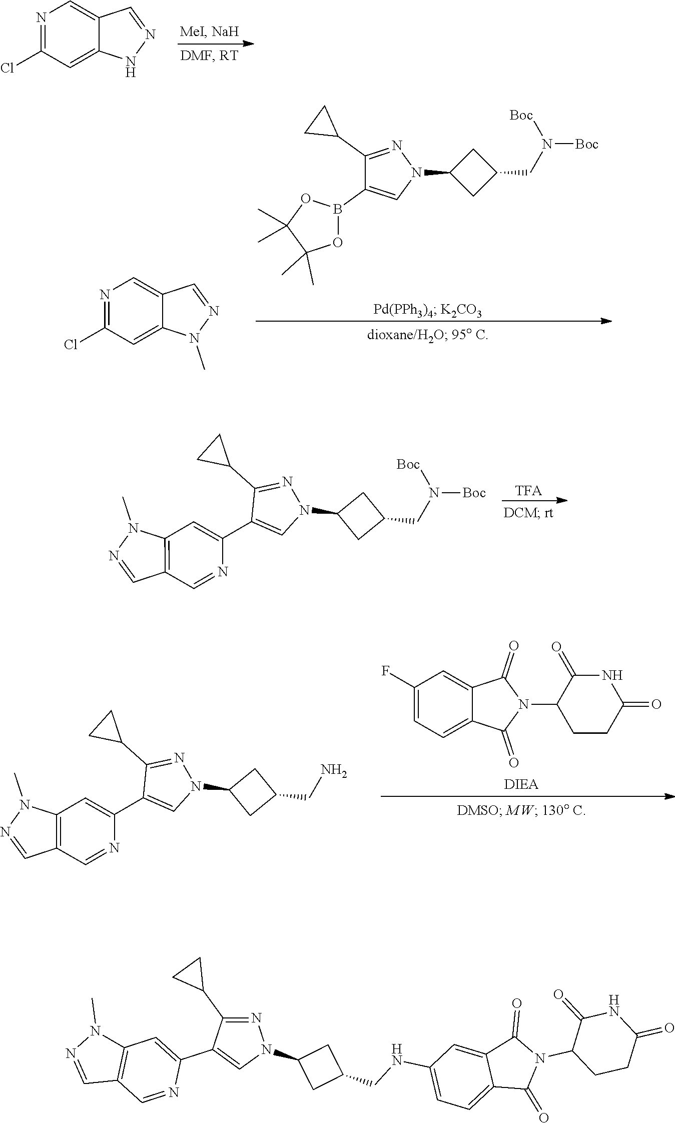

Step 1. Synthesis of 6-chloro-1-methyl-1H-pyrazolo[4,3-c]pyridine

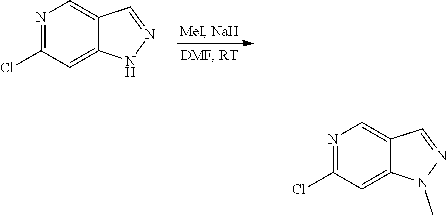

To a solution of 6-chloro-1H-pyrazolo[4,3-c]pyridine (600 mg, 3.91 mmol) in DMF (10 mL) was added NaH (312.56 mg, 7.81 mmol, 60% purity) at rt, the reaction mixture was stirred at rt for 2 min, then MeI (1.11 g, 7.81 mmol) was added dropwise to above mixture and stirred for 30 min. The reaction mixture was poured into water and extracted with EtOAc. The organic layers were combined and washed with brined, dried over Na₂SO₄, filtered, and concentrated. The resulting residue was purified by silica gel flash chromatography (petroleum ether/EtOAc) to provide the title compound (414 mg, 63.2% yield) as a white solid. MS (ESI) m/z=168.2 [M+H]⁺.

Step 2. Synthesis of trans-tert-butyl N-tert-butoxycarbonyl-N-[[3-[3-cyclopropyl-4-(1-methylpyrazolo[4,3-c]pyridin-6-yl)pyrazol-1-yl]cyclobutyl]methyl]carbamate

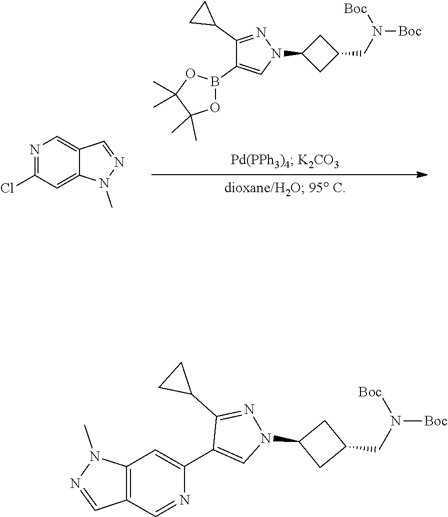 [SMILES: Cn1ncc2cnc(Cl)cc21 ; CC(C)(C)OC(=O)N(C[C@H]1C[C@H](n2cc(B3OC(C)(C)C(C)(C)O3)c(C3CC3)n2)C1)C(=O)OC(C)(C)C ; Cn1ncc2cnc(-c3cn([C@H]4C[C@H](CN(C(=O)OC(C)(C)C)C(=O)OC(C)(C)C)C4)nc3C3CC3)cc21]

To a solution of 6-chloro-1-methyl-1H-pyrazolo[4,3-c]pyridine (80 mg, 477 μmol) in dioxane (3 mL) and H₂O (0.6 mL) were added Pd(PPh₃)₄ (55 mg, 48 μmol), K₂CO₃ (197.62 mg, 1.43 mmol) and trans-tert-butyl N-tert-butoxycarbonyl-N-[[3-[3-cyclopropyl-4-(4,4,5,5-tetramethyl-1,3,2-dioxaborolan-2-yl)pyrazol-1-yl]cyclobutyl]methyl]carbamate (247 mg, 477 μmol). The resulting mixture was stirred at 100° C. for 12 h, then it was poured into water and extracted with (EA/MeOH=10:1). The combined organic layers were dried over Na₂SO₄, filtered, and concentrated. The resulting residue was purified by prep-TLC (petroleum ether/EtOAc=3:2) to provide the title compound (23 mg, 9.2% yield) as a colorless oil. MS (ESI) m/z=523.8 [M+H]⁺.

Step 3. Synthesis of (trans-3-(3-cyclopropyl-4-(1-methyl-1H-pyrazolo[4,3-c]pyridin-6-yl)-1H-pyrazol-1-yl)cyclobutyl)methanamine

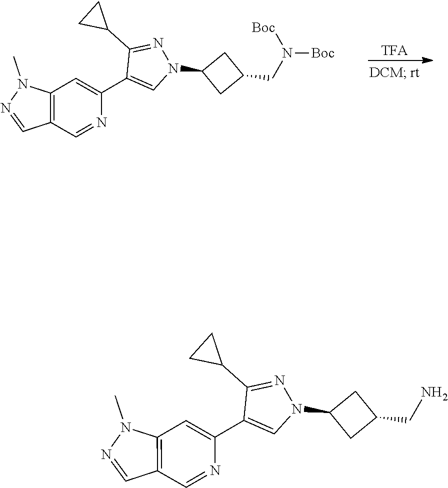 [SMILES: Cn1ncc2cnc(-c3cn([C@H]4C[C@H](CN(C(=O)OC(C)(C)C)C(=O)OC(C)(C)C)C4)nc3C3CC3)cc21 ; Cn1ncc2cnc(-c3cn([C@H]4C[C@H](CN)C4)nc3C3CC3)cc21]

The mixture of trans-tert-butyl N-tert-butoxycarbonyl-N-[[3-[3-cyclopropyl-4-(1-methylpyrazolo[4,3-c]pyridin-6-yl)pyrazol-1-yl]cyclobutyl]methyl]carbamate (23 mg, 44 μmol) and TFA (1 mL) in DCM (2 mL) was stirred at rt for 1 h, then it was concentrated to provide the crude product (14 mg, 98.7% yield) which was used directly in the next step without further purification. MS (ESI) m/z=323.5 [M+H]⁺.

Step 4. Synthesis of 5-(((trans-3-(3-cyclopropyl-4-(1-methyl-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione

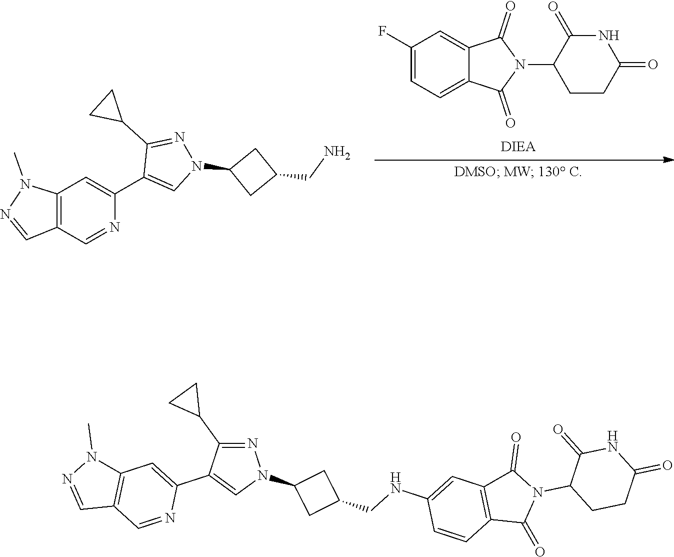

The mixture of (trans-3-(3-cyclopropyl-4-(1-methyl-1H-pyrazolo[4,3-c]pyridin-6-yl)-1H-pyrazol-1-yl)cyclobutyl)methanamine (14 mg, 43.42 μmol), 2-(2,6-dioxo-3-piperidyl)-5-fluoro-isoindoline-1,3-dione (23.99 mg, 86.85 μmol) and DIEA (112.03 mg, 868.47 μmol) in DMSO (0.7 mL) was stirred at 130° C. for 2 h under microwave. The mixture was purified by reverse phase column chromatography, followed by prep-TLC (DCM/MeOH=13:1) to provide the title compound (6.3 mg, 25.1% yield) as a yellow solid. MS (ESI) m/z=579.7 [M+H]⁺.

### Example 600. 5-(((trans-3-(3-Cyclopropyl-4-(1-isopropyl-1H-pyrazolo[4,3-c]pyridin-6-yl)-1H-pyrazol-1-yl)cyclobutyl)methyl)amino)-2-(2,6-dioxopiperidin-3-yl)isoindoline-1,3-dione (GS-614)

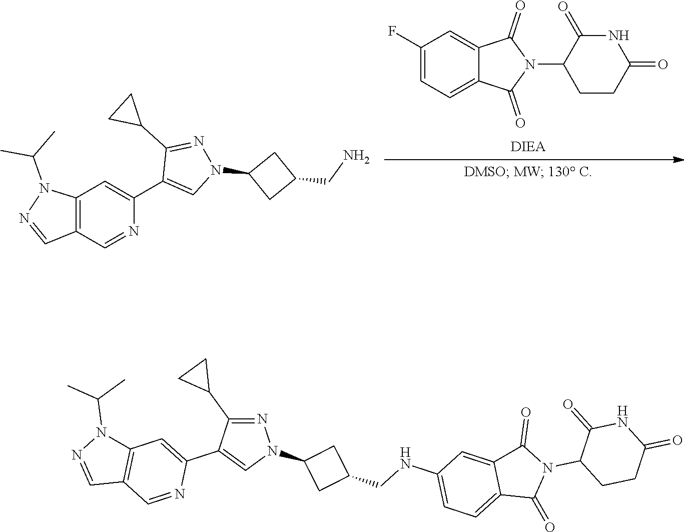

GS-614 was synthesized following the similar procedure for preparing GS-613 (6.1 mg, yield: 20.7%). MS (ESI) m/z=607.8 [M+H]⁻.

### Example 601. 5-(((trans-3-(3-Cyclopropyl-4-(1-methyl-1H-pyrrolo[3,2-c]pyridin-4-yl)-1H-pyrazol-1-yl)cyclobutyl)methyl)amino)-2-(2,6-dioxopiperidin-3-yl)isoindoline-1,3-dione (GS-615)

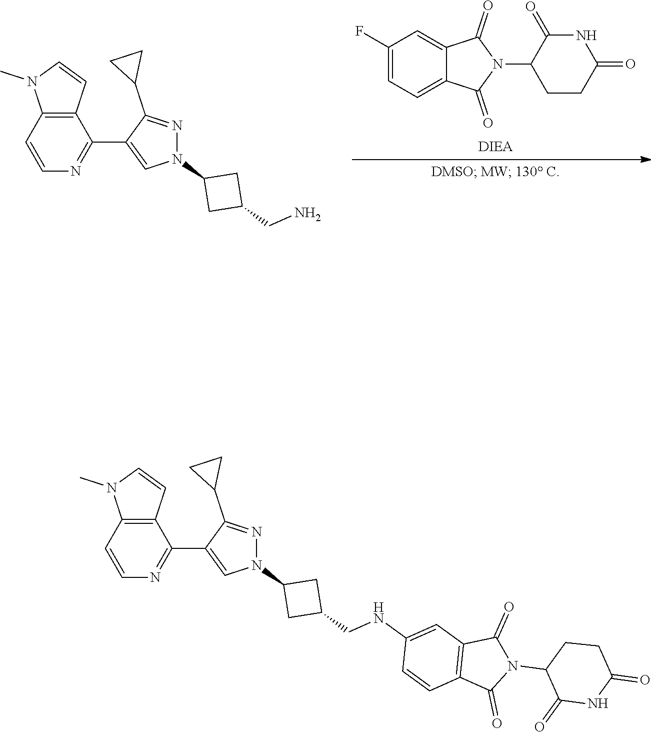 [SMILES: Cn1ccc2c(-c3cn([C@H]4C[C@H](CN)C4)nc3C3CC3)nccc21 ; O=C1CCC(N2C(=O)c3ccc(F)cc3C2=O)C(=O)N1 ; Cn1ccc2c(-c3cn([C@H]4C[C@H](CNc5ccc6c(c5)C(=O)N(C5CCC(=O)NC5=O)C6=O)C4)nc3C3CC3)nccc21]

GS-615 was synthesized following the similar procedure for preparing GS-613 (7.7 mg, yield: 14.2%). MS (ESI) m/z=578.8 [M+H]⁻.

### Example 602. 5-(((trans-3-(3-Cyclopropyl-4-(6,7-dihydro-5H-cyclopenta[b]pyridin-2-yl)-1H-pyrazol-1-yl)cyclobutyl)methyl)amino)-2-(2,6-dioxopiperidin-3-yl)isoindoline-1,3-dione (GS-616)

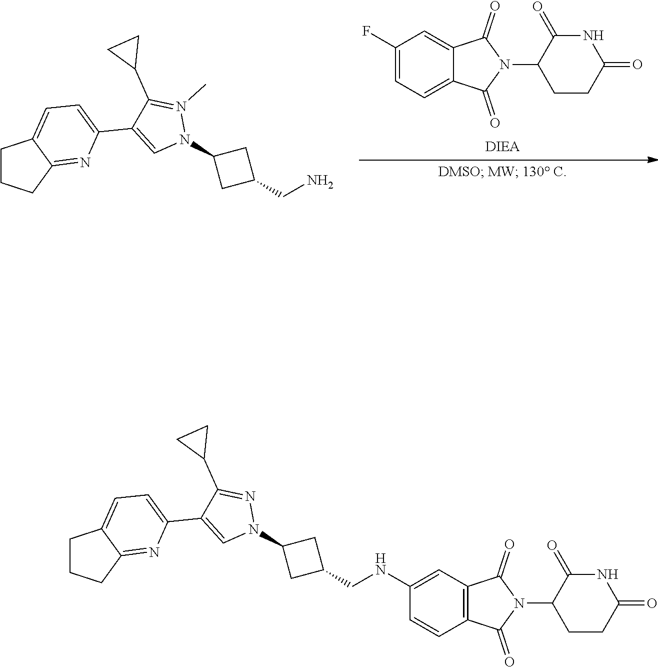

GS-616 was synthesized following the similar procedure for preparing GS-604 (16.4 mg, yield: 72.5%). MS (ESI) m/z=565.8 [M+H]⁻.

### Example 603. 5-(((trans-3-(3-Cyclopropyl-4-(3-((1-methylpiperidin-4-yl)amino)pyridin-2-yl)-1H-pyrazol-1-yl)cyclobutyl)methyl)amino)-2-(2,6-dioxopiperidin-3-yl)isoindoline-1,3-dione GS-617)

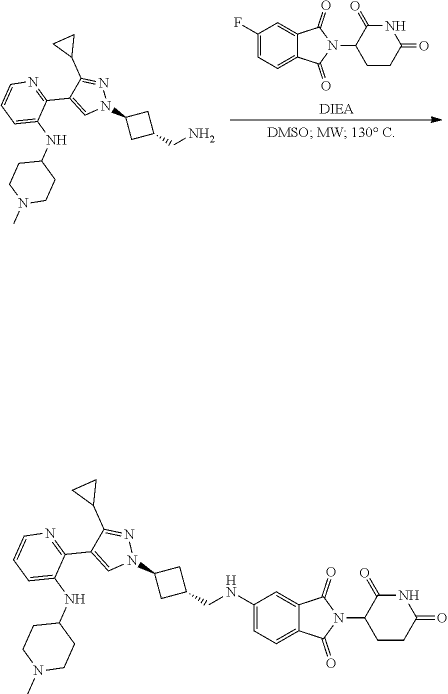 [SMILES: CN1CCC(Nc2cccnc2-c2cn([C@H]3C[C@H](CN)C3)nc2C2CC2)CC1 ; O=C1CCC(N2C(=O)c3ccc(F)cc3C2=O)C(=O)N1 ; CN1CCC(Nc2cccnc2-c2cn([C@H]3C[C@H](CNc4ccc5c(c4)C(=O)N(C4CCC(=O)NC4=O)C5=O)C3)nc2C2CC2)CC1]

GS-617 was synthesized following the similar procedure for preparing GS-604 (1.5 mg, yield: 10.5%). MS (ESI) m/z=637.6 [M+H]⁻.

### Example 604. 5-(((trans-3-(3-Cyclopropyl-4-(1-methyl-1H-pyrrolo[3,2-c]pyridin-6-yl)-1H-pyrazol-1-yl)cyclobutyl)methyl)amino)-2-(2,6-dioxopiperidin-3-yl)isoindoline-1,3-dione (GS-618)

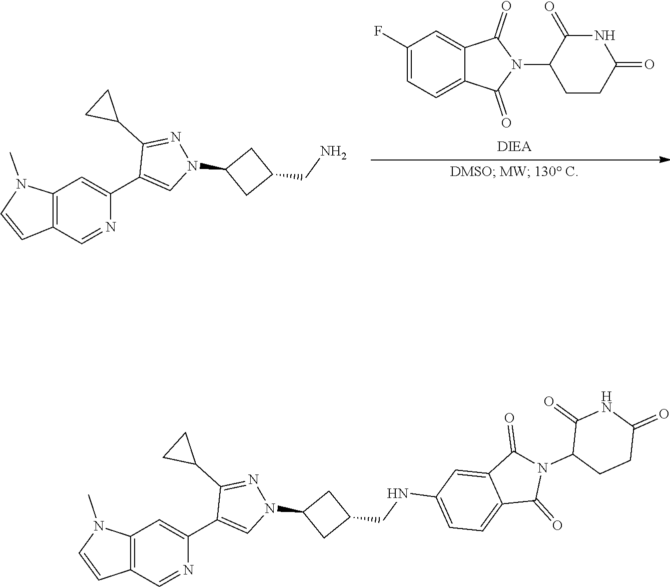

GS-618 was synthesized following the similar procedure for preparing GS-613 (2.1 mg, yield: 4.7%). MS (ESI) m/z=578.7 [M+H]⁺.

### Example 605. 5-(((trans-3-(3-Cyclopropyl-4-(6-(1-methylazetidin-3-yl)pyridin-2-yl)-1H-pyrazol-1-yl)cyclobutyl)methyl)amino)-2-(2,6-dioxopiperidin-3-yl)isoindoline-1,3-dione (GS-619)

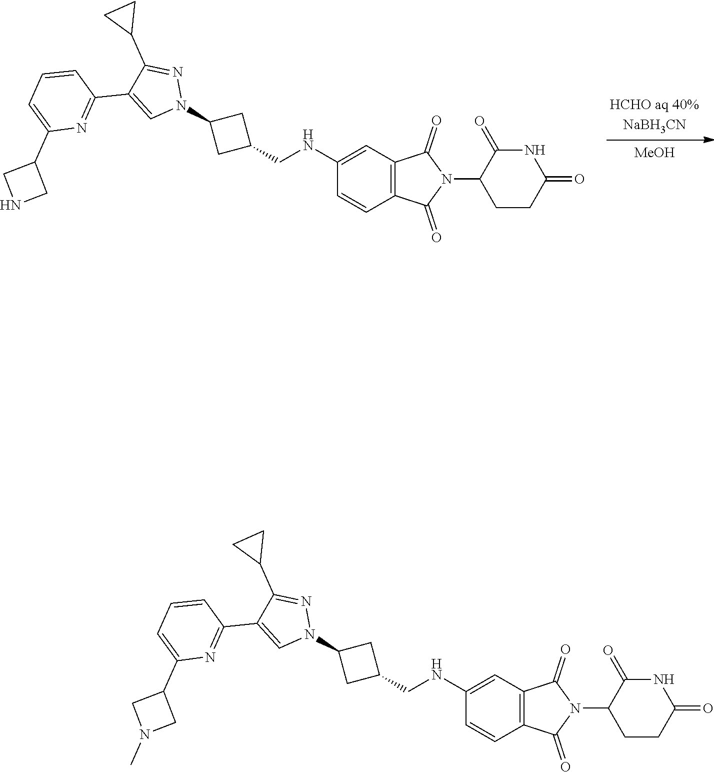 [SMILES: "MeOH" ; O=C1CCC(N2C(=O)c3ccc(NC[C@H]4C[C@H](n5cc(-c6cccc(C7CNC7)n6)c(C6CC6)n5)C4)cc3C2=O)C(=O)N1 ; CN1CC(c2cccc(-c3cn([C@H]4C[C@H](CNc5ccc6c(c5)C(=O)N(C5CCC(=O)NC5=O)C6=O)C4)nc3C3CC3)n2)C1]

To a solution of 5-(((trans-3-(4-(6-(azetidin-3-yl)pyridin-2-yl)-3-cyclopropyl-1H-pyrazol-1-yl)cyclobutyl)methyl)amino)-2-(2,6-dioxopiperidin-3-yl)isoindoline-1,3-dione (12 mg, 17.30 μmol, TF) and HCHO aq 40% (103.80 μmol, 0.3 mL) in MeOH (0.9 mL) was added NaBH₃CN (10.90 mg, 172.99 μmol). The reaction mixture was stirred at rt for 12 h, before it was purified by reverse phase column chromatography to provide the title compound (2.3 mg, 22.4% yield) as a yellow solid. MS (ESI) m/z=594.7 [M+H]⁺.

### Example 606. 5-(((trans-3-(3-Cyclopropyl-4-(3-(methylamino)pyridin-2-yl)-1H-pyrazol-1-yl)cyclobutyl)methyl)amino)-2-(2,6-dioxopiperidin-3-yl)isoindoline-1,3-dione (GS-620)

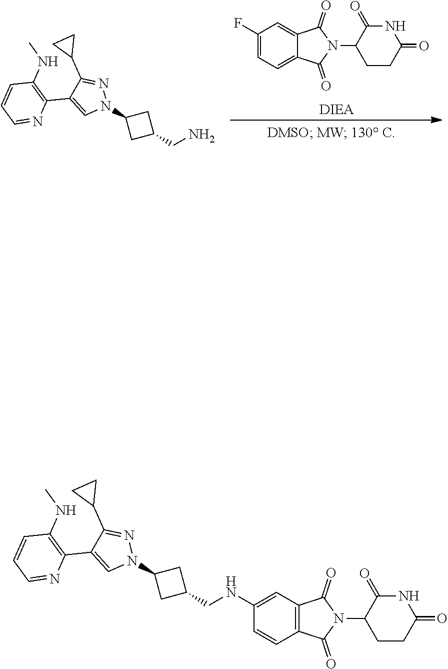 [SMILES: CNc1cccnc1-c1cn([C@H]2C[C@H](CN)C2)nc1C1CC1 ; O=C1CCC(N2C(=O)c3ccc(F)cc3C2=O)C(=O)N1 ; CNc1cccnc1-c1cn([C@H]2C[C@H](CNc3ccc4c(c3)C(=O)N(C3CCC(=O)NC3=O)C4=O)C2)nc1C1CC1]

GS-620 was synthesized following the similar procedure for preparing GS-604 (10.2 mg, yield: 8.7%). MS (ESI) m/z=554.6 [M+H]⁺.

### Example 607. 5-(((trans-3-(3-Cyclopropyl-4-(3-(dimethylamino)pyridin-2-yl)-1H-pyrazol-1-yl)cyclobutyl)methyl)amino)-2-(2,6-dioxopiperidin-3-yl)isoindoline-1,3-dione (GS-621)

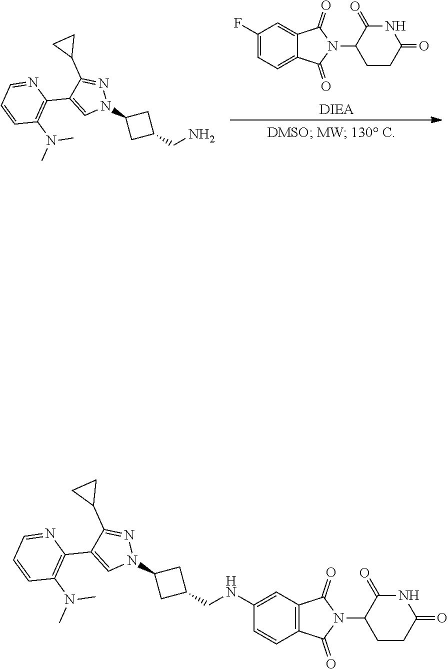 [SMILES: CN(C)c1cccnc1-c1cn([C@H]2C[C@H](CN)C2)nc1C1CC1 ; O=C1CCC(N2C(=O)c3ccc(F)cc3C2=O)C(=O)N1 ; CN(C)c1cccnc1-c1cn([C@H]2C[C@H](CNc3ccc4c(c3)C(=O)N(C3CCC(=O)NC3=O)C4=O)C2)nc1C1CC1]

GS-621 was synthesized following the similar procedure for preparing GS-604 (8.9 mg, yield: 35%). MS (ESI) m/z=568.8 [M+H]⁺.

### Example 608. 5-(((trans-3-(3-Cyclopropyl-4-(1-isopropyl-1H-pyrrolo[3,2-c]pyridin-6-yl)-1H-pyrazol-1-yl)cyclobutyl)methyl)amino)-2-(2,6-dioxopiperidin-3-yl)isoindoline-1,3-dione (GS-622)

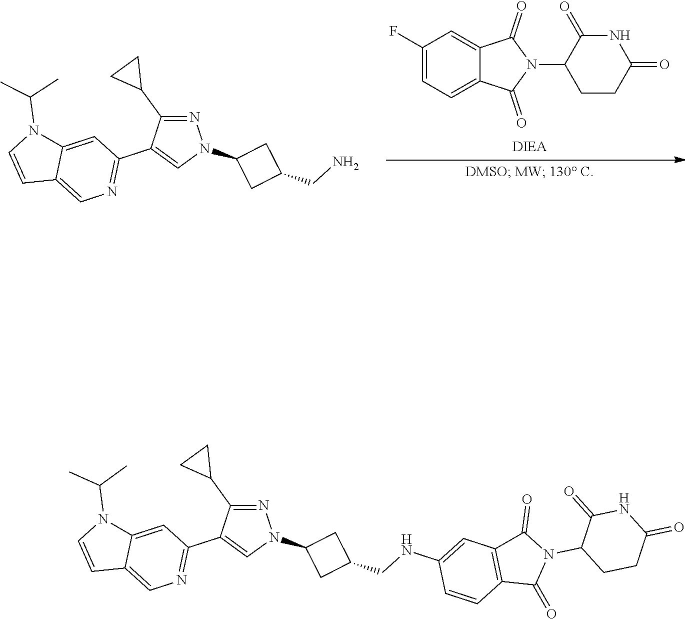

GS-622 was synthesized following the similar procedure for preparing GS-613 (15 mg, yield: 14.5%). MS (ESI) m/z=606.8 [M+H]⁺.

### Example 609. 5-(((trans-3-(3-Cyclopropyl-4-(1-isopropyl-1H-pyrrolo[3,2-c]pyridin-4-yl)-1H-pyrazol-1-yl)cyclobutyl)methyl)amino)-2-(2,6-dioxopiperidin-3-yl)isoindoline-1,3-dione (GS-623)

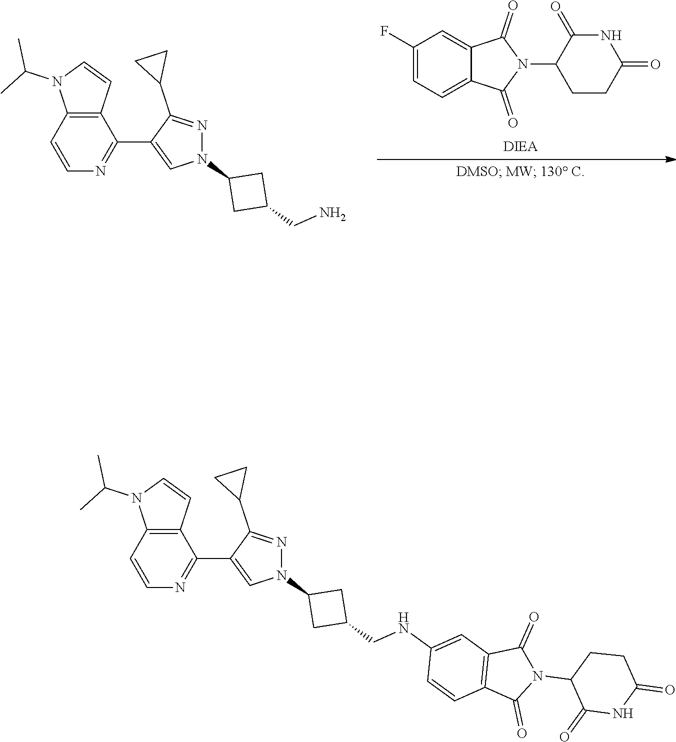 [SMILES: CC(C)n1ccc2c(-c3cn([C@H]4C[C@H](CN)C4)nc3C3CC3)nccc21 ; O=C1CCC(N2C(=O)c3ccc(F)cc3C2=O)C(=O)N1 ; CC(C)n1ccc2c(-c3cn([C@H]4C[C@H](CNc5ccc6c(c5)C(=O)N(C5CCC(=O)NC5=O)C6=O)C4)nc3C3CC3)nccc21]

GS-623 was synthesized following the similar procedure or preparing GS-613 (22.4 mg, yield: 51.7%). MS (ESI) m/z=606.8 [M+H]⁺.

### Example 610. 5-(((trans-3-(4-(5-(Azetidin-3-yl)quinoxalin-2-yl)-3-cyclopropyl-1H-pyrazol-1-yl)cyclobutyl)methyl)amino)-2-(2,6-dioxopiperidin-3-yl)isoindoline-1,3-dione (GS-624)

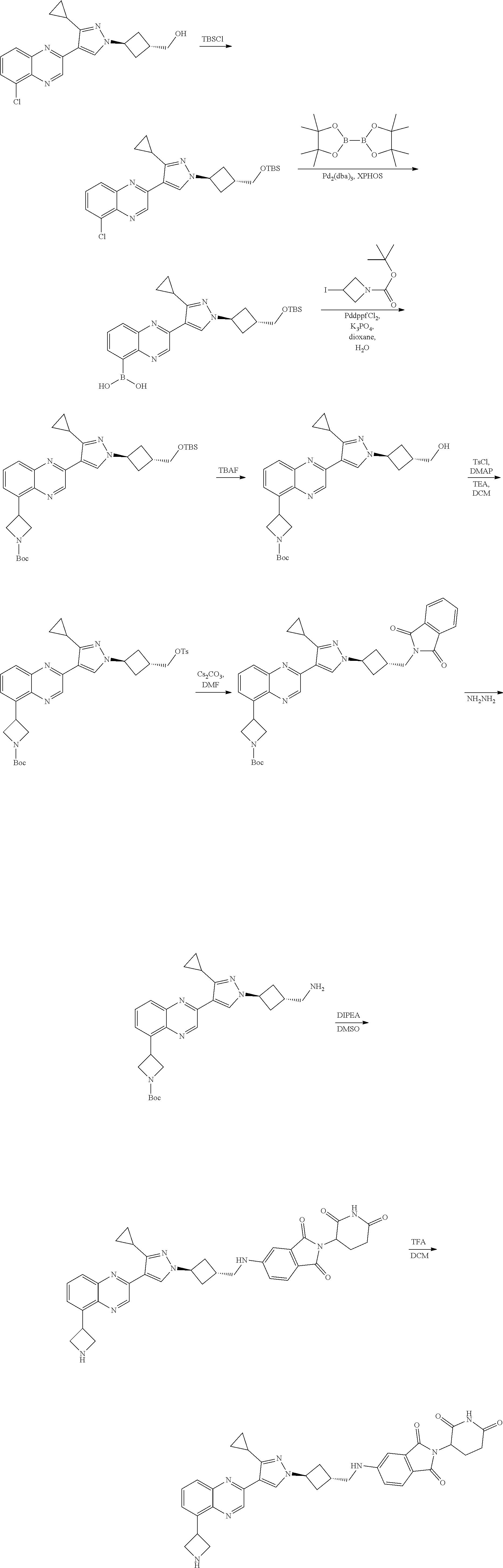

Step 1. Synthesis of 2-(1-(trans-3-(((tert-butyldimethylsilyl)oxy)methyl)cyclobutyl)-3-cyclopropyl-1H-pyrazol-4-yl)-5-chloroquinoxaline

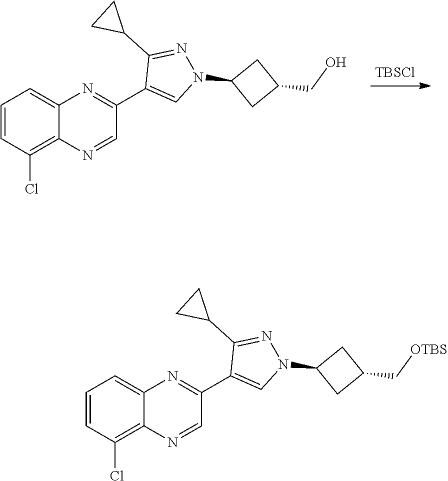

To a solution of (trans-3-(4-(5-chloroquinoxalin-2-yl)-3-cyclopropyl-1H-pyrazol-1-yl)cyclobutyl)methanol (1.0 g, 2.82 mmol) in DCM (20 mL) were added imidazole (766.56 mg, 11.27 mmol) and tert-butyl-chloro-dimethyl-silane (849.53 mg, 5.64 mmol). After the reaction was stirred at rt for 3 h, it was concentrated and the residue was purified by silica gel chromatography (petroleum ether/EtOAc=10/1) to provide the title compound (1.1 g, 83.2% yield) as a yellow oil. MS (ESI) m/z=469.7 [M+H]⁺.

Step 2. Synthesis of (2-(1-(trans-3-(((tert-butyldimethylsilyl)oxy)methyl)cyclobutyl)-3-cyclopropyl-1H-pyrazol-4-yl)quinoxalin-5-yl)boronic acid

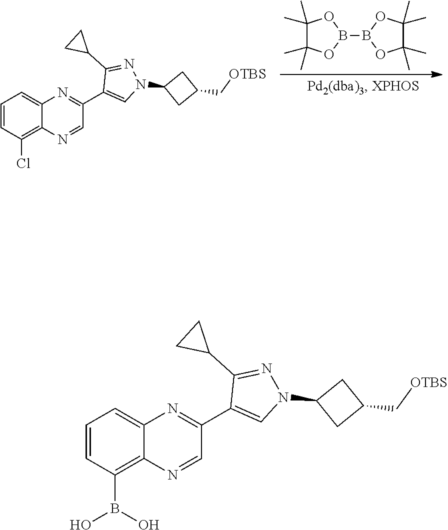

To a solution of 2-(1-(trans-3-(((tert-butyldimethylsilyl)oxy)methyl)cyclobutyl)-3-cyclopropyl-1H-pyrazol-4-yl)-5-chloroquinoxaline (3.4 g, 7.25 mmol) and 4,4,5,5-tetramethyl-2-(4,4,5,5-tetramethyl-1,3,2-dioxaborolan-2-yl)-1,3,2-dioxaborolane (3.68 g, 14.50 mmol) in dioxane (100 mL) were added Pd₂(dba)₃ (663.19 mg, 724.80 μmol), Xphos (690.01 mg, 1.45 mmol) and AcOK (2.13 g, 21.74 mmol). The resulting mixture was stirred at 100° C. for 16 h, before it was concentrated. The resulting residue was purified by silica gel chromatography (petroleum ether/EtOAc=10/1 to 5/1) to provide the title compound (2.1 g, 60.6% yield). MS (ESI) m/z=479.6 [M+H]⁺.

Step 3. Synthesis of tert-butyl 3-(2-(1-(trans-3-(((tert-butyldimethylsilyl)oxy)methyl)cyclobutyl)-3-cyclopropyl-1H-pyrazol-4-yl)quinoxalin-5-yl)azetidine-1-carboxylate

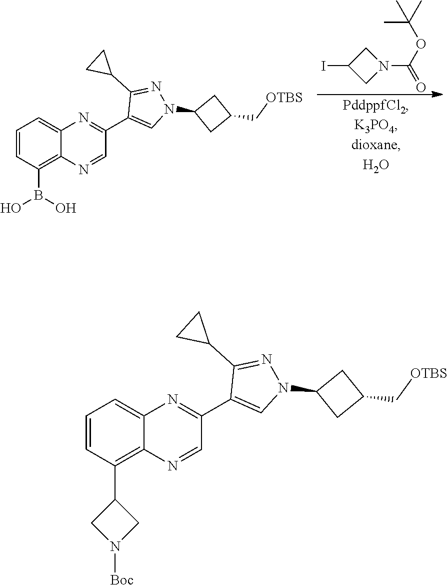 [SMILES: CC(C)(C)[Si](C)(C)OC[C@H]1C[C@H](n2cc(-c3cnc4c(B(O)O)cccc4n3)c(C3CC3)n2)C1 ; CC(C)(C)OC(=O)N1CC(I)C1 ; "PddppfCl2,K3PO4,dioxane,H2O" ; CC(C)(C)OC(=O)N1CC(c2cccc3nc(-c4cn([C@H]5C[C@H](CO[Si](C)(C)C(C)(C)C)C5)nc4C4CC4)cnc23)C1]

To a solution of (2-(1-(trans-3-(((tert-butyldimethylsilyl)oxy)methyl)cyclobutyl)-3-cyclopropyl-1H-pyrazol-4-yl)quinoxalin-5-yl)boronic acid (2.1 g, 4.39 mmol) and tert-butyl 3-iodoazetidine-1-carboxylate (4.97 g, 17.56 mmol) in dioxane (80 mL) and water (20 mL) were added Pd(dppf)Cl₂ (320.84 mg, 438.90 μmol) and K₃PO₄ (3.72 g, 17.56 mmol), the reaction mixture was stirred at 100° C. for 3 h, before it was cooled to rt, concentrated and purified by silica gel chromatography (petroleum ether/EtOAc=20/1 to 10/1) to provide the title compound (370 mg, 14.3% yield). MS (ESI) m/z=590.8 [M+H]⁺.

Step 4. Synthesis of tert-butyl 3-(2-(3-cyclopropyl-1-(trans-3-(hydroxymethyl)cyclobutyl)-1H-pyrazol-4-yl)quinoxalin-5-yl)azetidine-1-carboxylate

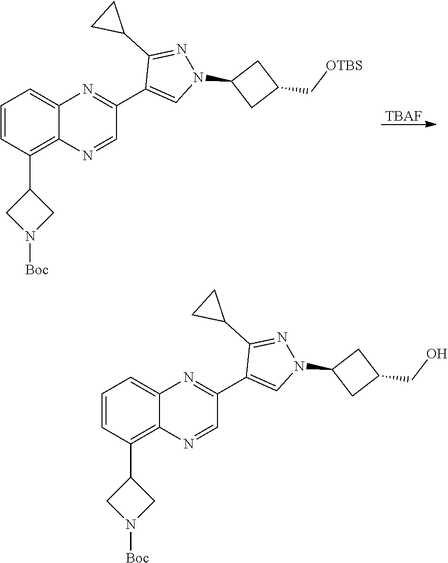

To a solution of tert-butyl 3-(2-(1-(trans-3-(((tert-butyldimethylsilyl)oxy)methyl)cyclobutyl)-3-cyclopropyl-1H-pyrazol-4-yl)quinoxalin-5-yl)azetidine-1-carboxylate (420 mg, 712.06 μmol) in THF (10 mL) was added TBAF (1M in THF, 2 mL). After the reaction was stirred at rt. for 2 h, it was concentrated. The resulting residue was purified by silica gel chromatography (petroleum ether/EtOAc=5/1 to 1/1) to provide the title compound (300 mg, 88.59% yield). MS (ESI) m/z=476.8 [M+H]⁺.

Step 5. Synthesis of 5-(((trans-3-(4-(5-(azetidin-3-yl)quinoxalin-2-yl)-3-cyclopropyl-1H-pyrazol-1-yl)cyclobutyl)methyl)amino)-2-(2,6-dioxopiperidin-3-yl)isoindoline-1,3-dione

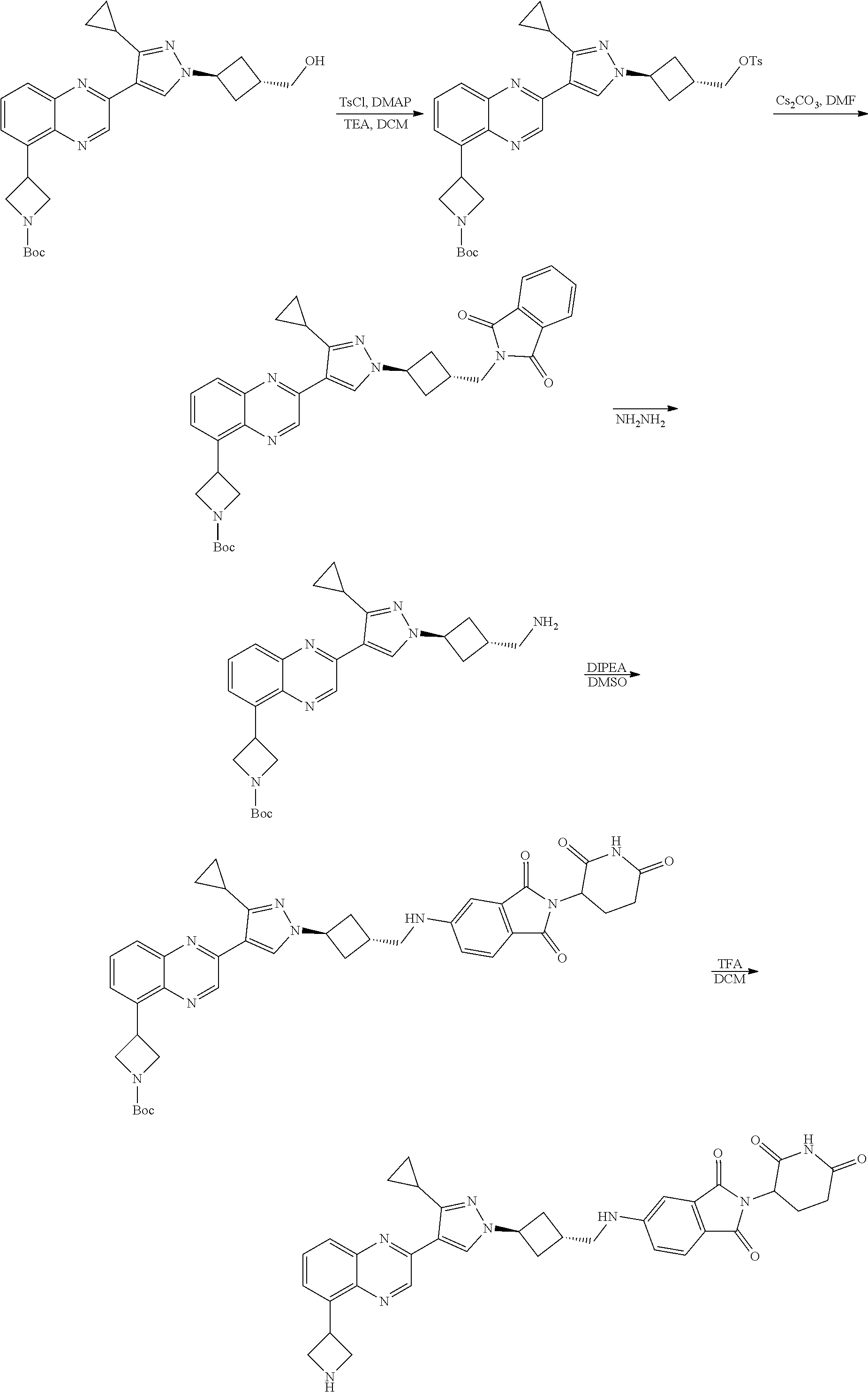

GS-624 was synthesized following the similar procedure for preparing GS-605 (15 mg, yield: 18.6%). MS (ESI) m/z=631.7 [M+H]⁺.

### Example 611. 5-((3-(trans-3-(3-Cyclopropyl-4-(1,7-naphthyridin-8-yl)-1H-pyrazol-1-yl)cyclobutyl)propyl)amino)-2-(2,6-dioxopiperidin-3-yl)isoindoline-1,3-dione (GS-625)

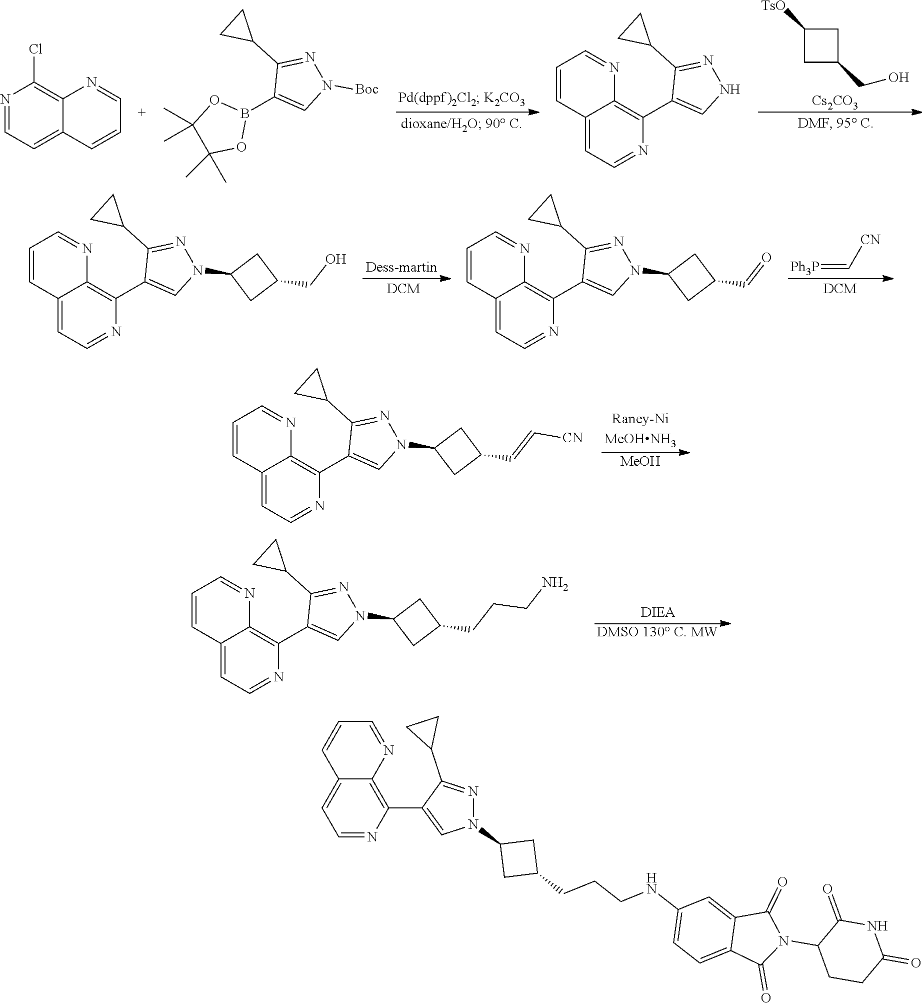

Step 1. Synthesis of 8-(3-cyclopropyl-H-pyrazol-4-yl)-1,7-naphthyridine

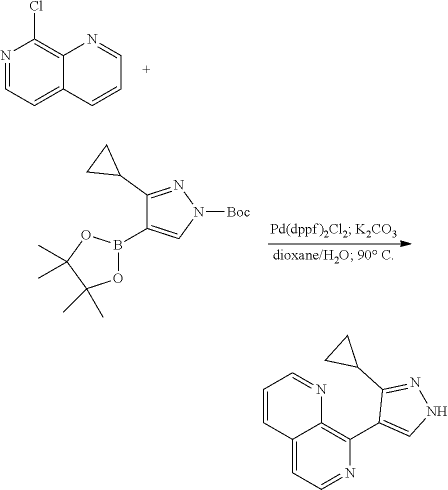

To a solution of 8-chloro-1,7-naphthyridine (500 mg, 3.04 mmol) in dioxane (15 mL) and H₂O (5 mL) were added Pd(dppf)₂Cl₂ (222 mg, 304 μmol), K₂CO₃ (2.10 g, 15.19 mmol) and tert-butyl 3-cyclopropyl-4-(4,4,5,5-tetramethyl-1,3,2-dioxaborolan-2-yl)pyrazole-1-carboxylate (1.12 g, 3.34 mmol). The mixture was stirred at 90° C. for 12 h, before it was poured into water and extracted with EtOAc. The combined organic layers were dried over Na₂SO₄, filtered, and concentrated. The resulting residue was purified by reverse phase column chromatography to provide the title compounds (348 mg, 48.5% yield) as a black oil. MS (ESI) m/z=237.4 [M+H]⁺.

Step 2. Synthesis of (trans-3-(3-cyclopropyl-4-(1,7-naphthyridin-8-yl)-1H-pyrazol-1-yl)cyclobutyl)methanol

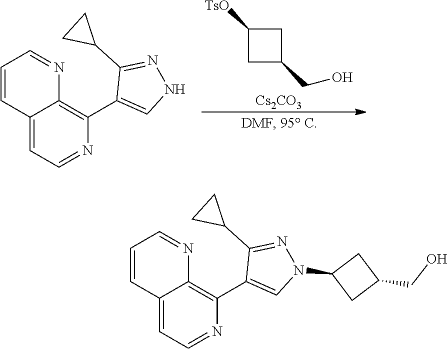

To a solution of 8-(3-cyclopropyl-1H-pyrazol-4-yl)-1,7-naphthyridine (348 mg, 1.47 mmol) in DMF (5 mL) were added cis-3-(hydroxymethyl)cyclobutyl 4-methylbenzenesulfonate (679 mg, 2.65 mmol) and Cs₂CO₃ (1.44 g, 4.42 mmol). The mixture was stirred at 95° C. for 6 h, before it was purified by reverse phase column chromatography, followed by prep-TLC (DCM/MeOH=12:1) to provide the title compound (202 mg, 42.8% yield) as a white solid. MS (ESI) m/z=321.6 [M+H]⁺.

Step 3. Synthesis of trans-3-(3-cyclopropyl-4-(1,7-naphthyridin-8-yl)-1H-pyrazol-1-yl)cyclobutane-1-carbaldehyde

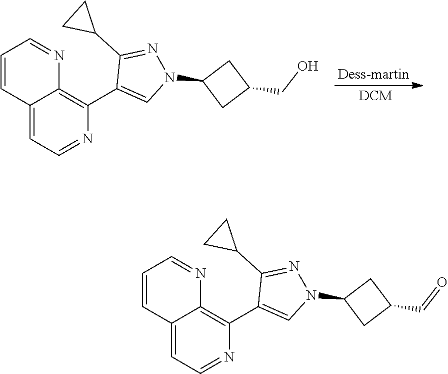 [SMILES: OC[C@H]1C[C@H](n2cc(-c3nccc4cccnc34)c(C3CC3)n2)C1 ; O=C[C@H]1C[C@H](n2cc(-c3nccc4cccnc34)c(C3CC3)n2)C1]

To a solution of (trans-3-(3-cyclopropyl-4-(1,7-naphthyridin-8-yl)-1H-pyrazol-1-yl)cyclobutyl)methanol (202 mg, 630.49 μmol) in DCM (5 mL) was added Dess-Martin Periodinane (535 mg, 1.26 mmol). After the mixture was stirred at rt for 1 h, it was filtered and washed with DCM and EtOAc. The filtrate was concentrated to provide the title compound (200 mg, 99.6% yield) as a brown oil. MS (ESI) m/z=319.4 [M+H]⁺.

Step 4. Synthesis of (E)-3-(trans-3-(3-cyclopropyl-4-(1,7-naphthyridin-8-yl)-1H-pyrazol-1-yl)cyclobutyl)acrylonitrile

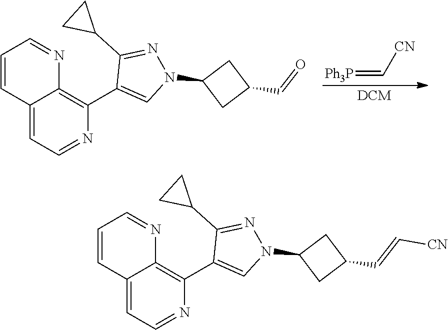

To a solution of trans-3-(3-cyclopropyl-4-(1,7-naphthyridin-8-yl)-1H-pyrazol-1-yl)cyclobutane-1-carbaldehyde (200 mg, 628 μmol) in DCM (8 mL) was added 2-(triphenyl-phosphanylidene)acetonitrile (341 mg, 1.13 mmol). The resulting mixture was stirred at rt for 0.5 h, before it was concentrated and purified by reverse phase column to provide the title compound (248 mg, 86.7% yield) as a white solid. MS (ESI) m/z=342.5 [M+H]⁺.

Step 5. Synthesis of 3-(trans-3-(3-cyclopropyl-4-(1,7-naphthyridin-8-yl)-1H-pyrazol-1-yl)cyclobutyl)propan-1-amine

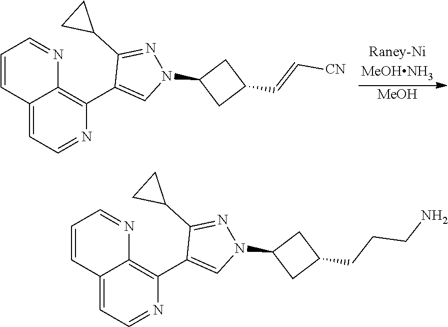 [SMILES: "MeOH" ; N#C/C=C/[C@H]1C[C@H](n2cc(-c3nccc4cccnc34)c(C3CC3)n2)C1 ; NCCC[C@H]1C[C@H](n2cc(-c3nccc4cccnc34)c(C3CC3)n2)C1]

To a solution of (E)-3-(trans-3-(3-cyclopropyl-4-(1,7-naphthyridin-8-yl)-1H-pyrazol-1-yl)cyclobutyl)acrylonitrile (12 mg, 35 μmol) in MeOH (1 mL) were added NH₃/MeOH (2 mL) and Raney-Ni (5 mg). The reaction mixture was stirred under hydrogen atmosphere at rt for 0.5 h, before it was filtered, and concentrated to provide the title compound (10 mg, 60.6% yield) as a white solid. MS (ESI) m/z=348.5 [M+H]⁺.

Step 6. Synthesis of 5-((3-(trans-3-(3-cyclopropyl-4-(1,7-naphthyridin-8-yl)-1H-pyrazol-1-yl)cyclobutyl)propyl)amino)-2-(2,6-dioxopiperidin-3-yl)isoindoline-1,3-dione

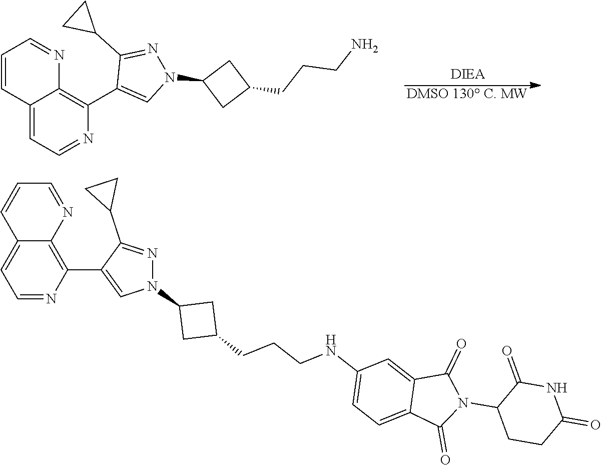

The mixture of 3-(trans-3-(3-cyclopropyl-4-(1,7-naphthyridin-8-yl)-1H-pyrazol-1-yl)cyclobutyl)propan-1-amine (10 mg, 21.3 μmol), 2-(2,6-dioxo-3-piperidyl)-5-fluoro-isoindoline-1,3-dione (12 mg, 43 μmol) and DIEA (55 mg, 426 μmol) in DMSO (0.6 mL) was irradiated at 130° C. for 0.7 h in a microwave reactor. The reaction was purified by reverse phase column chromatography, followed by prep-TLC (DCM/MeOH=13:1) to provide the title compound (4.4 mg, 34.2% yield) as a yellow solid. MS (ESI) m/z=604.8 [M+H]⁺.

### Example 612. 5-((3-(trans-3-(3-Cyclopropyl-4-(1H-pyrazolo[4,3-c]pyridin-4-yl)-1H-pyrazol-1-yl)cyclobutyl)propyl)amino)-2-(2,6-dioxopiperidin-3-yl)isoindoline-1,3-dione (GS-626)

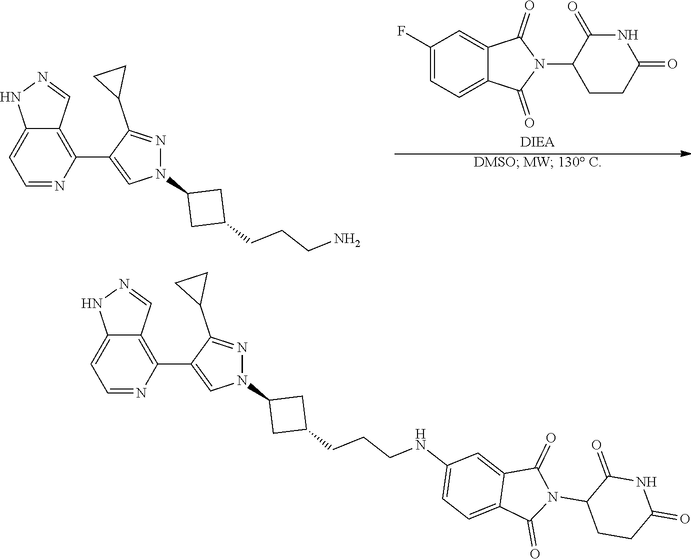 [SMILES: NCCC[C@H]1C[C@H](n2cc(-c3nccc4[nH]ncc34)c(C3CC3)n2)C1 ; O=C1CCC(N2C(=O)c3ccc(F)cc3C2=O)C(=O)N1 ; "DMSO; MW; 130° C." ; O=C1CCC(N2C(=O)c3ccc(NCCC[C@H]4C[C@H](n5cc(-c6nccc7[nH]ncc67)c(C6CC6)n5)C4)cc3C2=O)C(=O)N1]

GS-626 was synthesized following the similar procedure for preparing GS-625 (15 mg, yield: 68.5%). MS (ESI) m/z=593.8 [M+H]⁺.

### Example 613. 5-(2-(trans-3-(3-Cyclopropyl-4-(quinoxalin-2-yl)-1H-pyrazol-1-yl)cyclobutyl)ethyl)-2-(2,6-dioxopiperidin-3-yl)isoindoline-1,3-dione (GS-627)

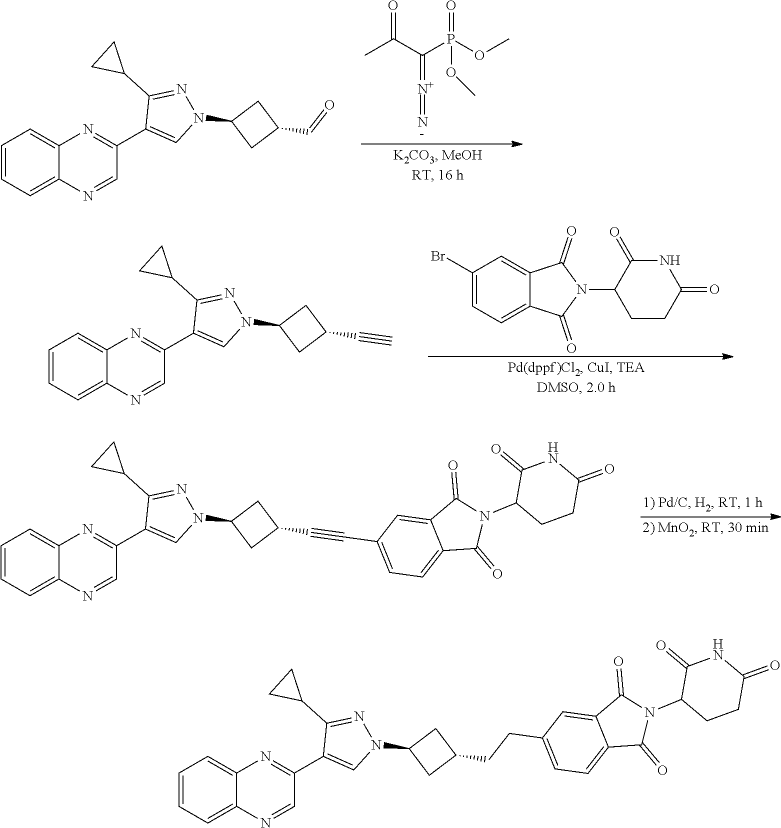 [SMILES: O=C[C@H]1C[C@H](n2cc(-c3cnc4ccccc4n3)c(C3CC3)n2)C1 ; COP(=O)(OC)C(=[N+]=N)C(C)=O ; C#C[C@H]1C[C@H](n2cc(-c3cnc4ccccc4n3)c(C3CC3)n2)C1 ; O=C1CCC(N2C(=O)c3ccc(Br)cc3C2=O)C(=O)N1 ; O=C1CCC(N2C(=O)c3ccc(C#C[C@H]4C[C@H](n5cc(-c6cnc7ccccc7n6)c(C6CC6)n5)C4)cc3C2=O)C(=O)N1 ; O=C1CCC(N2C(=O)c3ccc(CC[C@H]4C[C@H](n5cc(-c6cnc7ccccc7n6)c(C6CC6)n5)C4)cc3C2=O)C(=O)N1]

Step 1. Synthesis of 2-(3-cyclopropyl-1-(trans-3-ethynylcyclobutyl)-1H-pyrazol-4-yl)quinoxaline

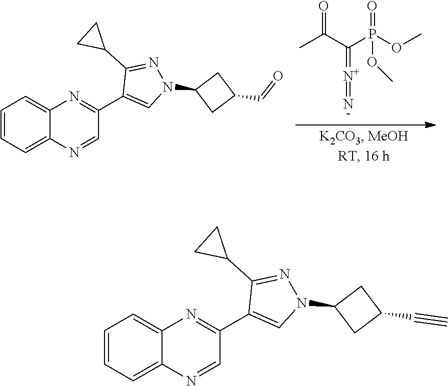 [SMILES: O=C[C@H]1C[C@H](n2cc(-c3cnc4ccccc4n3)c(C3CC3)n2)C1 ; COP(=O)(OC)C(=[N+]=N)C(C)=O ; C#C[C@H]1C[C@H](n2cc(-c3cnc4ccccc4n3)c(C3CC3)n2)C1]

To a solution of trans-3-(3-cyclopropyl-4-(quinoxalin-2-yl)-1H-pyrazol-1-yl)cyclobutane-1-carbaldehyde (205 mg, 643.90 μmol) and 1-diazo-1-dimethoxyphosphoryl-propan-2-one (148.44 mg, 772.68 μmol) in methanol (2.0 mL) was added K₂CO₃ (266.97 mg, 1.93 mmol). The reaction mixture was stirred at rt for 16 h, before it was poured into water and extracted with EtOAc (20 mL×2). The combined organic layers were washed with brine, dried over Na₂SO₄, filtered, and concentrated. The resulting residue was purified by column chromatography (EtOAc/petroleum ether=3:10) to provide the title compound (91 mg, 45% yield) as a white solid. MS (ESI) m/z=505.6 [M+H]⁺.

Step 2. Synthesis of 5-((trans-3-(3-cyclopropyl-4-(quinoxalin-2-yl)-1H-pyrazol-1-yl)cyclobutyl)ethynyl)-2-(2,6-dioxopiperidin-3-yl)isoindoline-1,3-dione

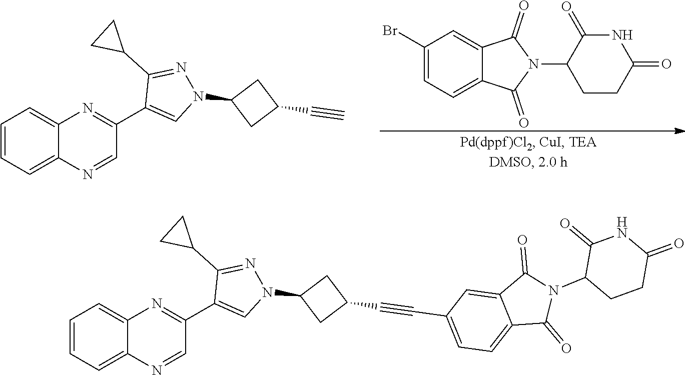 [SMILES: C#C[C@H]1C[C@H](n2cc(-c3cnc4ccccc4n3)c(C3CC3)n2)C1 ; O=C1CCC(N2C(=O)c3ccc(Br)cc3C2=O)C(=O)N1 ; O=C1CCC(N2C(=O)c3ccc(C#C[C@H]4C[C@H](n5cc(-c6cnc7ccccc7n6)c(C6CC6)n5)C4)cc3C2=O)C(=O)N1]

To a solution of 2-(3-cyclopropyl-1-(trans-3-ethynylcyclobutyl)-1H-pyrazol-4-yl)quinoxaline (91 mg, 289.46 μmol) and 5-bromo-2-(2,6-dioxo-3-piperidyl)isoindoline-1,3-dione (88.71 mg, 263.14 μmol) in DMSO (1.0 mL) were added Pd(dppf)Cl₂ (19.25 mg, 26.31 μmol), CuI (5.01 mg, 26.31 μmol) and TEA (266.27 mg, 2.63 mmol). The reaction mixture was stirred at 90° C. for 2 h, before it was purified by reverse phase column chromatography to provide the title compound (124.2 mg, 82.7% yield) as a yellow solid. MS (ESI) m/z=571.6 [M+H]$.

Step 3. Synthesis of 5-(2-(trans-3-(3-cyclopropyl-4-(quinoxalin-2-yl)-1H-pyrazol-1-yl)cyclobutyl)ethyl)-2-(2,6-dioxopiperidin-3-yl)isoindoline-1,3-dione

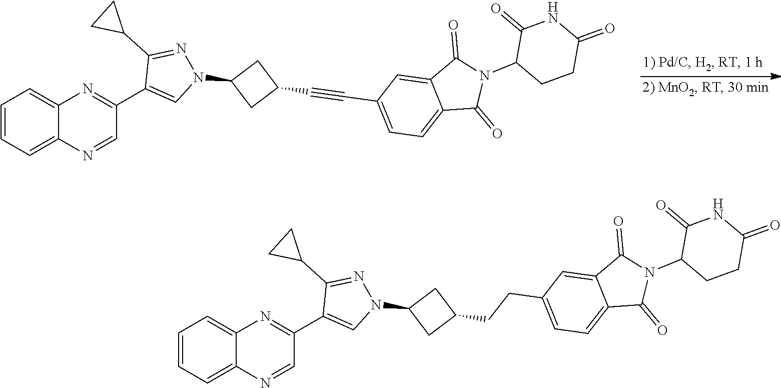 [SMILES: O=C1CCC(N2C(=O)c3ccc(C#C[C@H]4C[C@H](n5cc(-c6cnc7ccccc7n6)c(C6CC6)n5)C4)cc3C2=O)C(=O)N1 ; O=C1CCC(N2C(=O)c3ccc(CC[C@H]4C[C@H](n5cc(-c6cnc7ccccc7n6)c(C6CC6)n5)C4)cc3C2=O)C(=O)N1]

To a solution of 5-((trans-3-(3-cyclopropyl-4-(quinoxalin-2-yl)-1H-pyrazol-1-yl)cyclobutyl)ethynyl)-2-(2,6-dioxopiperidin-3-yl)isoindoline-1,3-dione (20 mg, 35.05 μmol) in isopropanol (2.0 mL) and EtOAc (0.5 mL) was added 10% Pd/C (20 mg). The reaction mixture was stirred at rt for 1 h under H₂, before it was filtered and the filtrate was concentrated. The resulting residue was dissolved in DCM (2.0 mL) and MnO₂ (60.94 mg, 701.02 μmol) was added. The reaction was stirred at rt for 30 min, filtered, and concentrated. The resulting residue was purified by reverse column chromatography to provide the title compound (7.9 mg, 39.3% yield) as a yellow solid. MS (ESI) m/z=575.7 [M+H]⁺.

### Example 614. 5-((3-(trans-3-(3-Cyclopropyl-4-(2,6-naphthyridin-1-yl)-1H-pyrazol-1-yl)cyclobutyl)propyl)amino)-2-(2,6-dioxopiperidin-3-yl)isoindoline-1,3-dione (GS-628)

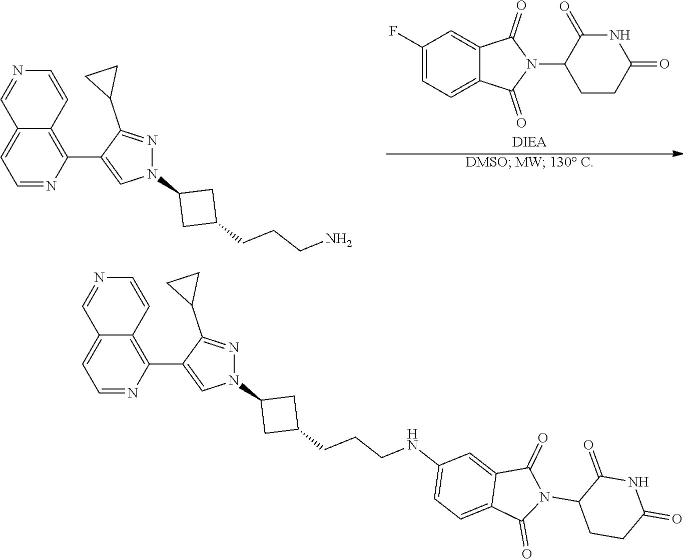 [SMILES: NCCC[C@H]1C[C@H](n2cc(-c3nccc4cnccc34)c(C3CC3)n2)C1 ; O=C1CCC(N2C(=O)c3ccc(F)cc3C2=O)C(=O)N1 ; "DMSO; MW; 130° C." ; O=C1CCC(N2C(=O)c3ccc(NCCC[C@H]4C[C@H](n5cc(-c6nccc7cnccc67)c(C6CC6)n5)C4)cc3C2=O)C(=O)N1]

GS-628 was synthesized following the similar procedure for preparing GS-625 (12.9 mg, yield: 12.2%). MS (ESI) m/z=604.7 [M+H]⁺.

### Example 615. 5-(2-(trans-3-(3-Cyclopropyl-4-(5-fluoro-6-methylpyridin-2-yl)-1H-pyrazol-1-yl)cyclobutyl)ethyl)-2-(2,6-dioxopiperidin-3-yl)isoindoline-1,3-dione (GS-629)

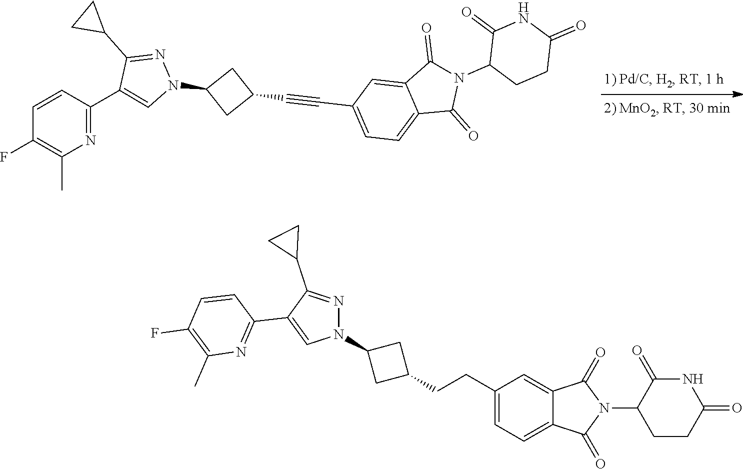 [SMILES: Cc1nc(-c2cn([C@H]3C[C@H](C#Cc4ccc5c(c4)C(=O)N(C4CCC(=O)NC4=O)C5=O)C3)nc2C2CC2)ccc1F ; Cc1nc(-c2cn([C@H]3C[C@H](CCc4ccc5c(c4)C(=O)N(C4CCC(=O)NC4=O)C5=O)C3)nc2C2CC2)ccc1F]

GS-629 was synthesized following the similar procedure for preparing GS-627 (12.2 mg, yield: 23.4%). MS (ESI) m/z=556.7 [M+H]⁺.

### Example 616. 5-((3-(trans-3-(3-Cyclopropyl-4-(2,7-naphthyridin-1-yl)-1H-pyrazol-1-yl)cyclobutyl)propyl)amino)-2-(2,6-dioxopiperidin-3-yl)isoindoline-1,3-dione (GS-630)

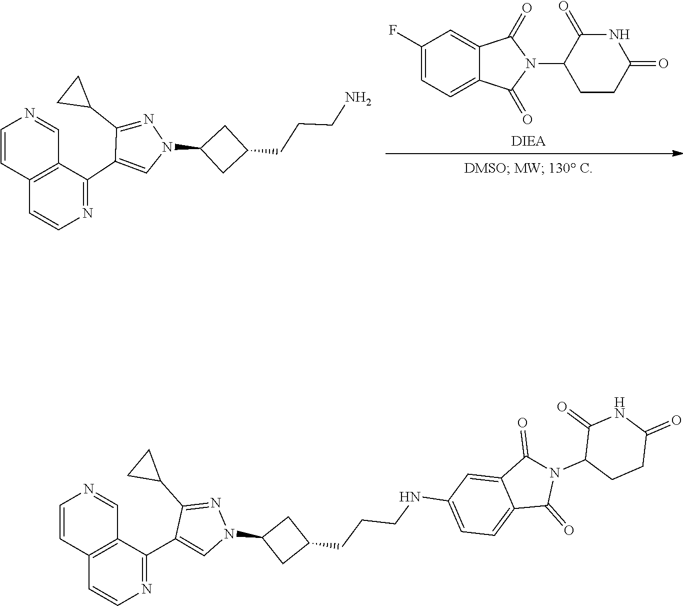 [SMILES: NCCC[C@H]1C[C@H](n2cc(-c3nccc4ccncc34)c(C3CC3)n2)C1 ; O=C1CCC(N2C(=O)c3ccc(F)cc3C2=O)C(=O)N1 ; O=C1CCC(N2C(=O)c3ccc(NCCC[C@H]4C[C@H](n5cc(-c6nccc7ccncc67)c(C6CC6)n5)C4)cc3C2=O)C(=O)N1]

GS-630 was synthesized following the similar procedure for preparing GS-625 (12.5 mg, yield: 19.9%). MS (ESI) m/z=604.7 [M+H]⁺.

### Example 617. 5-(((trans-3-(4-Cyclopentyl-3-cyclopropyl-1H-pyrazol-1-yl)cyclobutyl)methyl)amino)-2-(2,6-dioxopiperidin-3-yl)isoindoline-1,3-dione (GS-631)

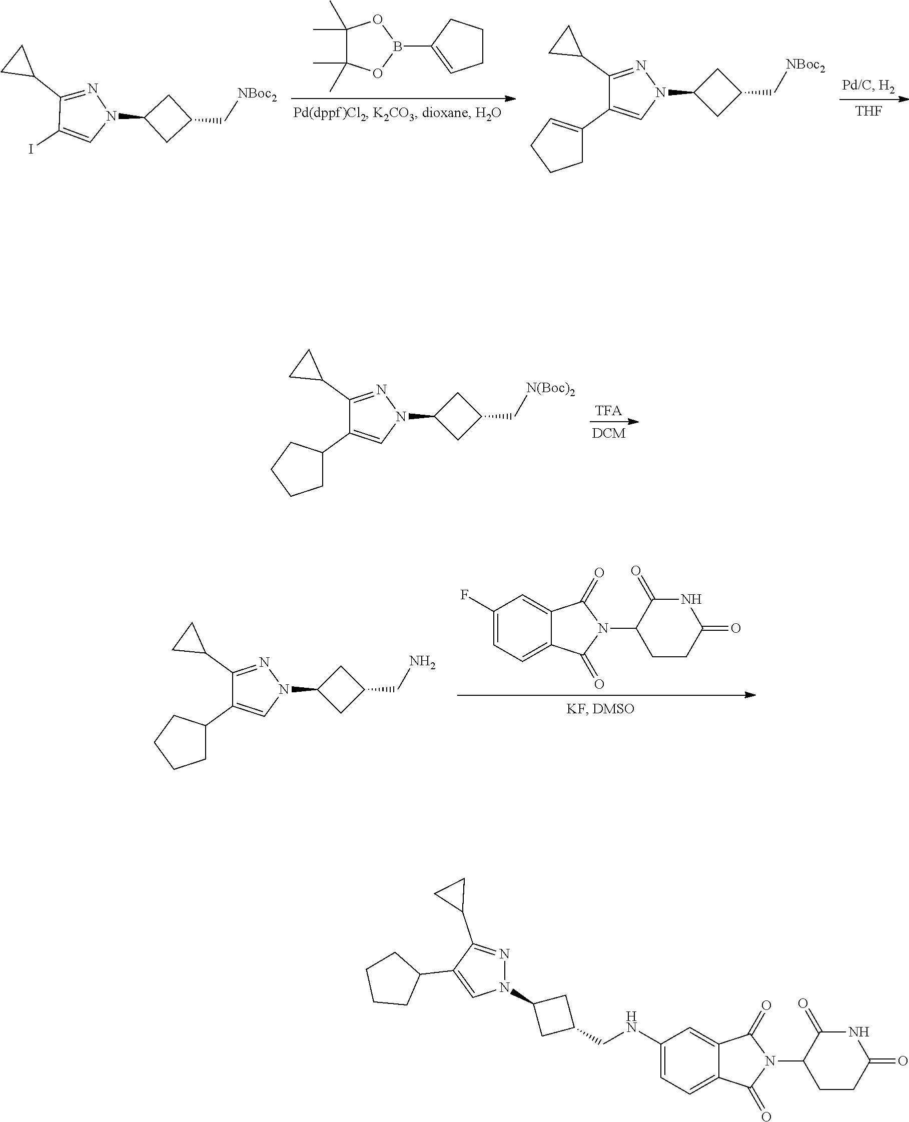

GS-631 (4.4 mg, 9% yield over 4 steps) was synthesized following the standard procedures for preparing GS-460 as a light-yellow solid. MS (ESI) m/z=516.3 [M+H]⁺.

### Example 618. 5-(((trans-3-(3,4-Dicyclopropyl-1H-pyrazol-1-yl)cyclobutyl)methyl)amino)-2-(2,6-dioxopiperidin-3-yl)isoindoline-1,3-dione (GS-632)

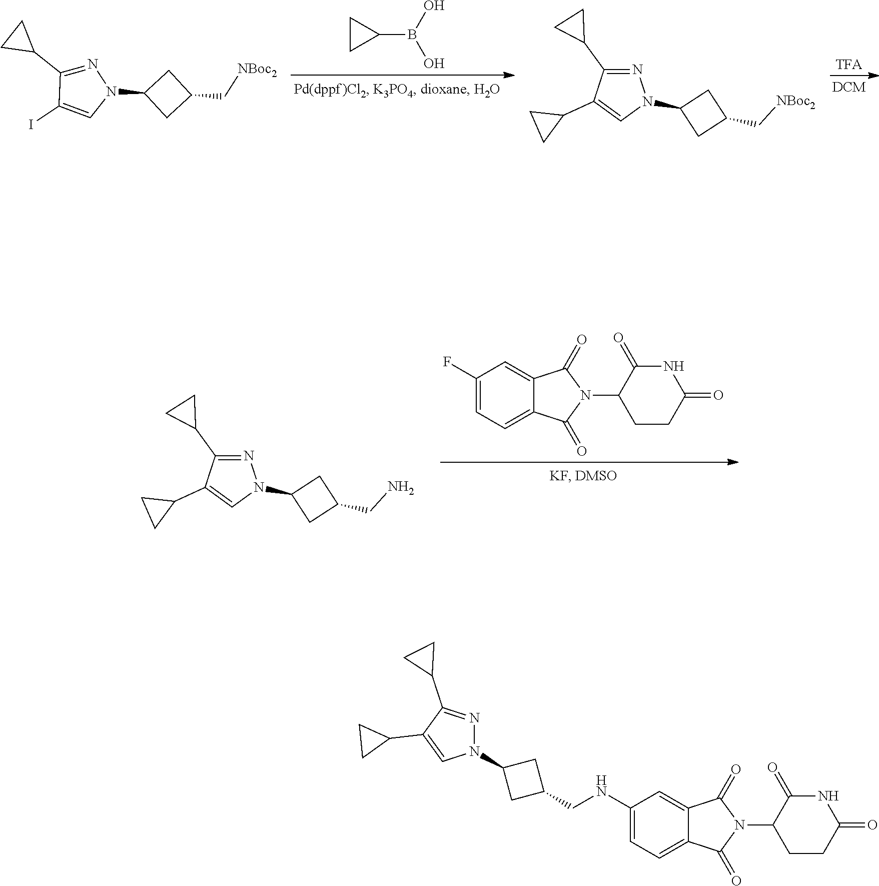

Step 1. Synthesis of tert-butyl N-tert-butoxycarbonyl-N-[[trans3-(3,4-dicyclopropylpyrazol-1-yl)cyclobutyl]methyl]carbamate

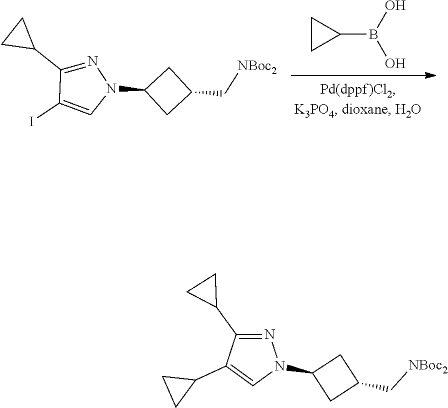

To a mixture of cyclopropylboronic acid (344.2 mg, 4.0 mmol) and tert-butyl-N-tert-butoxycarbonyl-N-[[trans-3-[3-cyclopropyl-4-iodo-pyrazol-1-yl]cyclobutyl]methyl]carbamate (207.2 mg, 0.4 mmol) in dioxane (9 mL) and H₂O (1 mL) were added K₃PO₄ (254.4 mg, 1.2 mmol) and Pd(dppf)Cl₂ (58.4 mg, 0.08 mmol) at rt. The reaction mixture was stirred at 80° C. for 2 h. After cooling down to rt, the solution was quenched with H₂O (10 mL) and extracted with ethyl acetate (10 mL×3). The combined organic layers were dried over anhydrous sodium sulfate, filtered, and concentrated under vacuum. The resulting residue was purified by reverse-phase chromatography to provide the desired product (20.2 mg, 12% yield) as a light-yellow solid. MS (ESI) m/z=432.3 [M+H]⁺.

GS-632 (1.5 mg, 7% yield over 2 steps) was then synthesized as a light-yellow solid following the standard procedures for preparing GS-438. MS (ESI) m/z=488.3 [M+H]⁺.

### Example 619. 5-(((trans-3-(3-Cyclopropyl-4-(2-methylcyclohexyl)-1H-pyrazol-1-yl)cyclobutyl)methyl)amino)-2-(2,6-dioxopiperidin-3-yl)isoindoline-1,3-dione (GS-633)

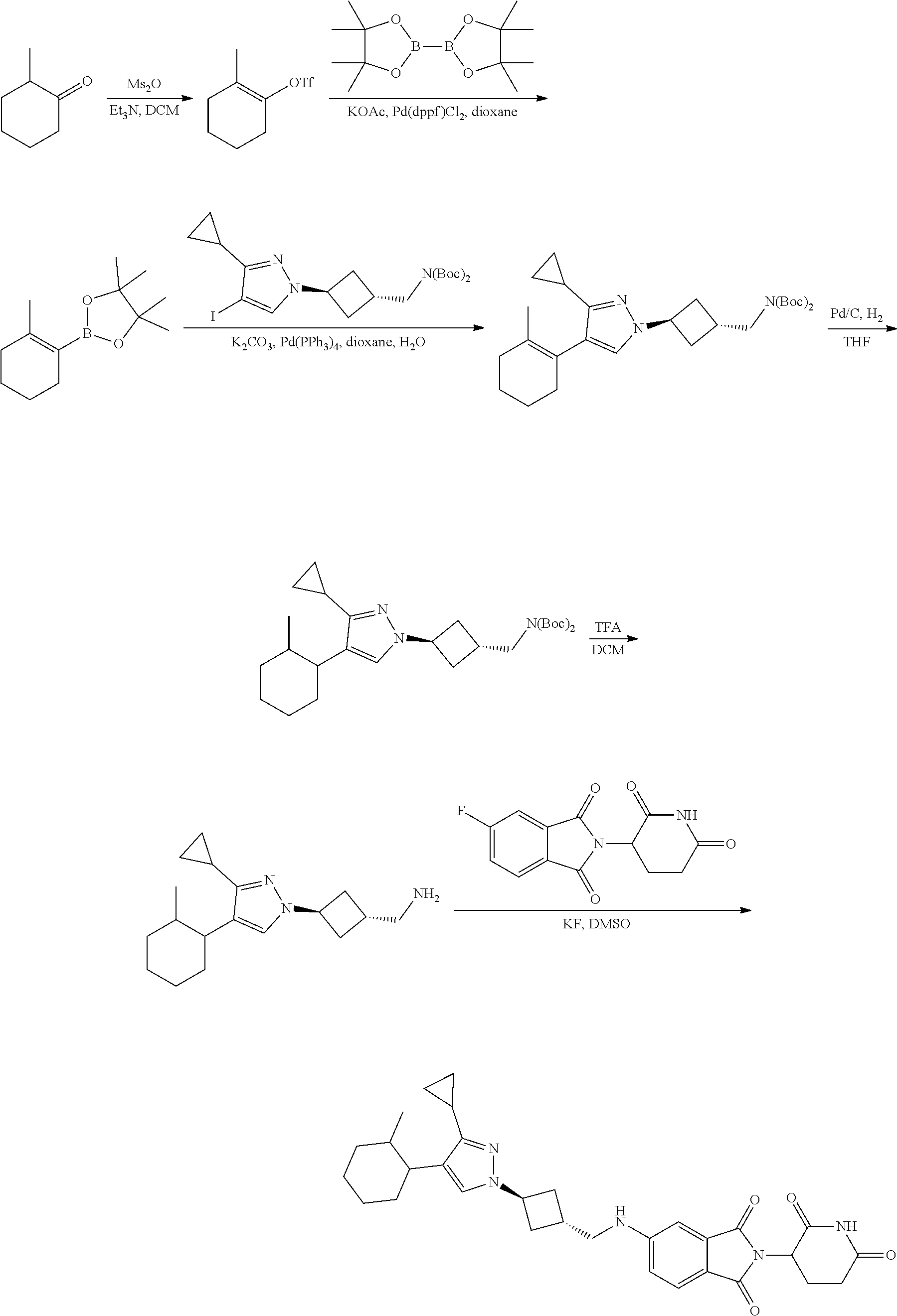

Step 1. Synthesis of 2-methylcyclohex-1-en-1-yl trifluoromethanesulfonate

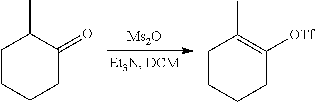

A mixture of 2-methylcyclohexan-1-one (3.0 g, 26.78 mmol) and triethylamine (5.4 g, 53.56 mmol) in DCM (50 mL) was stirred at 0° C. for 30 min. Then trifluoromethanesulfonic anhydride (7.55 g, 26.78 mmol) was added to the mixture dropwise at 0° C. After the reaction mixture was stirred at 0° C. for another 30 min, it was quenched with aq. sodium bicarbonate and extracted with DCM (2×50 mL). The combined organic layers were washed with saturated brine (50 mL), dried over anhydrous sodium sulfate and evaporated under reduced pressure. The resulting residue was purified by silica gel chromatography to provide the desired product (5.0 g, 76% yield) as a yellow oil.

Step 2. Synthesis of 4,4,5,5-tetramethyl-2-(2-methylcyclohex-1-en-1-yl)-1,3,2-dioxaborolane

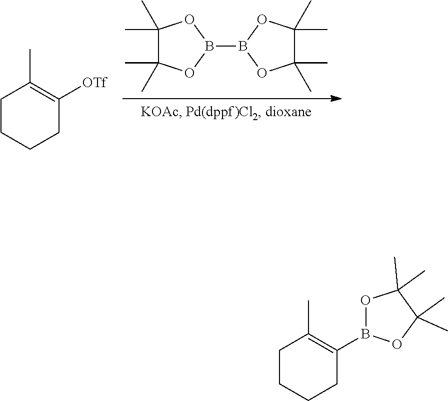

To a solution of 2-methylcyclohex-1-en-1-yl trifluoromethanesulfonate (2.0 g, 8.19 mmol) in dioxane (80 mL) were added 4,4,4′,4′,5,5,5′,5′-octamethyl-2,2′-bi(1,3,2-dioxaborolane) (2.49 g, 9.84 mmol), KOAc (2.26 g, 16.38 mmol) and Pd(dppf)Cl₂ (600 mg, 0.82 mmol). The reaction mixture was stirred at 90° C. under argon overnight. Then the reaction was quenched with water (30 mL) and extracted with ethyl acetate (3×50 mL). The combined organic layers were washed with saturated brine (50 mL), dried over anhydrous sodium sulfate and evaporated under reduced pressure. The resulting residue was purified by silica gel chromatography to provide the desired product (2.0 g, 76% yield) as a yellow oil.

Step 3. Synthesis of tert-butyl N-tert-butoxycarbonyl-N-[[trans-3-[3-cyclopropyl-4-(2-methylcyclohexen-1-yl)pyrazol-1-yl]cyclobutyl]methyl]carbamate

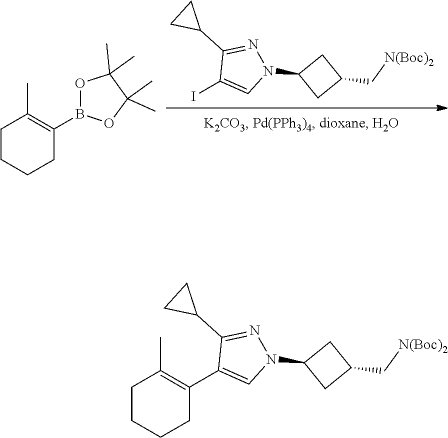

To a solution of 4,4,5,5-tetramethyl-2-(2-methylcyclohex-1-en-1-yl)-1,3,2-dioxaborolane (130 mg, 0.58 mmol) in dioxane (10 mL) and H₂O (2 mL) were added tert-butyl N-tert-butoxycarbonyl-N-[[trans-3-(3-cyclopropyl-4-iodo-pyrazol-1-yl)cyclobutyl]methyl]carbamate (100 mg, 0.19 mmol), K₂CO₃ (131 mg, 0.95 mmol) and Pd(PPh₃)₄ (22 mg, 0.019 mmol) at rt. The mixture was stirred at 90° C. under nitrogen for 3 h. After cooling down to rt, the reaction was quenched with water (50 mL) and extracted with ethyl acetate (3×50 mL). The combined organic layers were washed with saturated brine (50 mL), dried over anhydrous sodium sulfate and evaporated under reduced pressure. The resulting residue was purified by silica gel chromatography to provide the desired product (60 mg, 64% yield) as a yellow solid. MS (ESI) m/z=486.4 [M+H]⁺.

GS-633 (4.0 mg, 5.5% yield over 3 steps) was then synthesized as a yellow solid following the standard procedures for preparing GS-460. MS (ESI) m/z=544.3 [M+H]⁺.

### Example 620. 5-(((trans-3-(3-Cyclopropyl-4-(2-hydroxycyclohexyl)-1H-pyrazol-1-yl)cyclobutyl)methyl)amino)-2-(2,6-dioxopiperidin-3-yl)isoindoline-1,3-dione (GS-634)

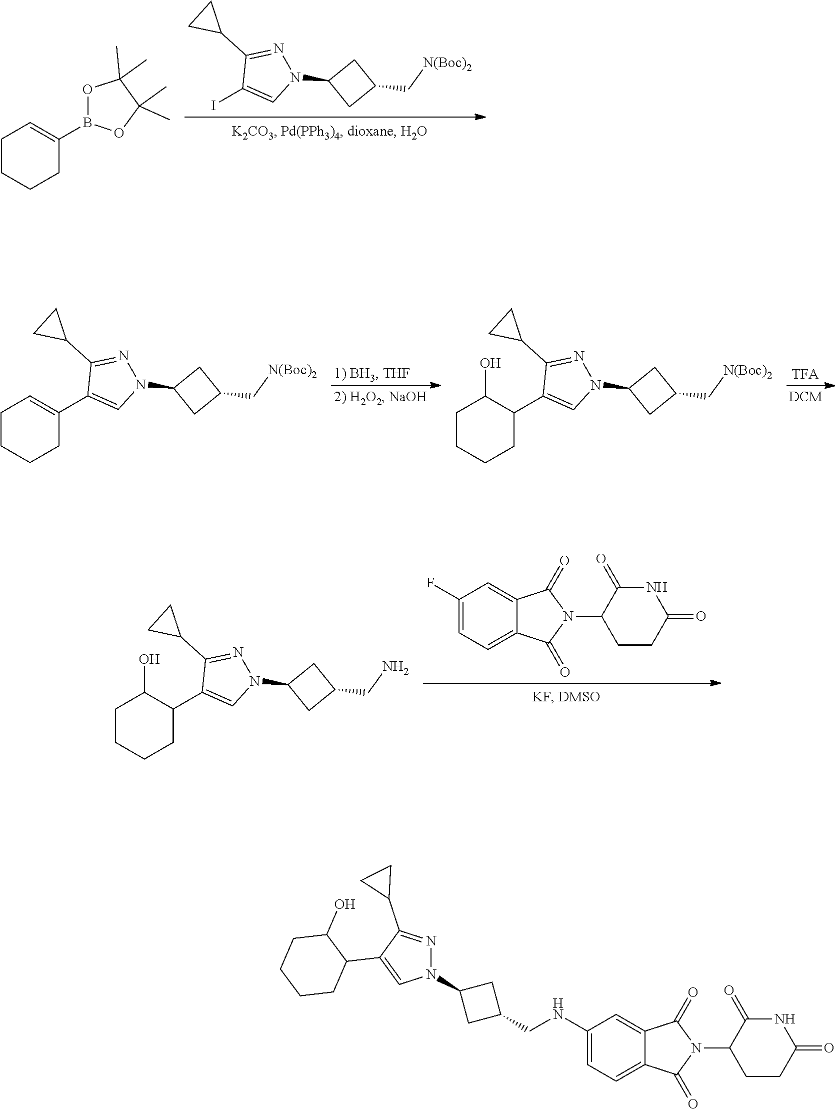

Step 1. Synthesis of tert-butyl N-tert-butoxycarbonyl-N-[[trans-3-[4-(cyclohexen-1-yl)-3-cyclopropyl-pyrazol-1-yl]cyclobutyl]methyl]carbamate

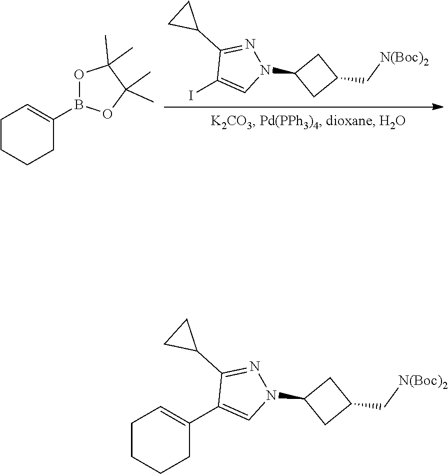

This step followed the standard procedure for preparing GS-633 to provide the desired product (35 mg, 38% yield) as a yellow solid. MS (ESI) m/z=472.4 [M+H]⁺.

Step 2. Synthesis of tert-butyl N-tert-butoxycarbonyl-N-[[trans-3-[3-cyclopropyl-4-(2-hydroxycyclohexyl)pyrazol-1-yl]cyclobutyl]methyl]carbamate

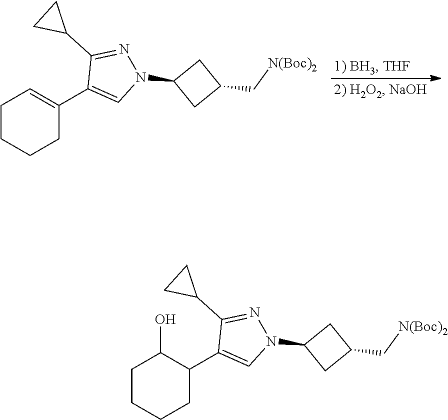

To a solution of tert-butyl N-tert-butoxycarbonyl-N-[[trans-3-[4-(cyclohexen-1-yl)-3-cyclopropyl-pyrazol-1-yl]cyclobutyl]methyl]carbamate (35 mg, 0.074 mmol) in THF (5 mL) was added BH₃ (1 M in THF, 0.4 mL) dropwise at 0° C. After the reaction was stirred at the same temperature for 3 h, 30% sodium hydroxide solution (1 mL) and H₂O₂ (1 mL) were added slowly. The reaction was stirred at 0° C. for another 30 min, before it was quenched with water (20 mL) and extracted with ethyl acetate (3×20 mL). The combined organic layers were washed with saturated brine (15 mL), dried over anhydrous sodium sulfate, and concentrated. The resulting residue was purified by silica gel chromatography to provide the desired product (20 mg, 55% yield) as a colorless oil. MS (ESI) m/z=490.3 [M+H]⁺. GS-634 (3.0 mg, 9% yield over 2 steps) was then synthesized as a yellow solid following the standard procedures for preparing GS-438. MS (ESI) m/z=544.3 [M+H]⁺.

### Example 621. 5-(((trans-3-(3-(Difluoromethyl)-4-(5-fluoro-6-methylpyridin-2-yl)-1H-pyrazol-1-yl)cyclobutyl)methyl)amino)-2-(2,6-dioxopiperidin-3-yl)isoindoline-1,3-dione (GS-635)

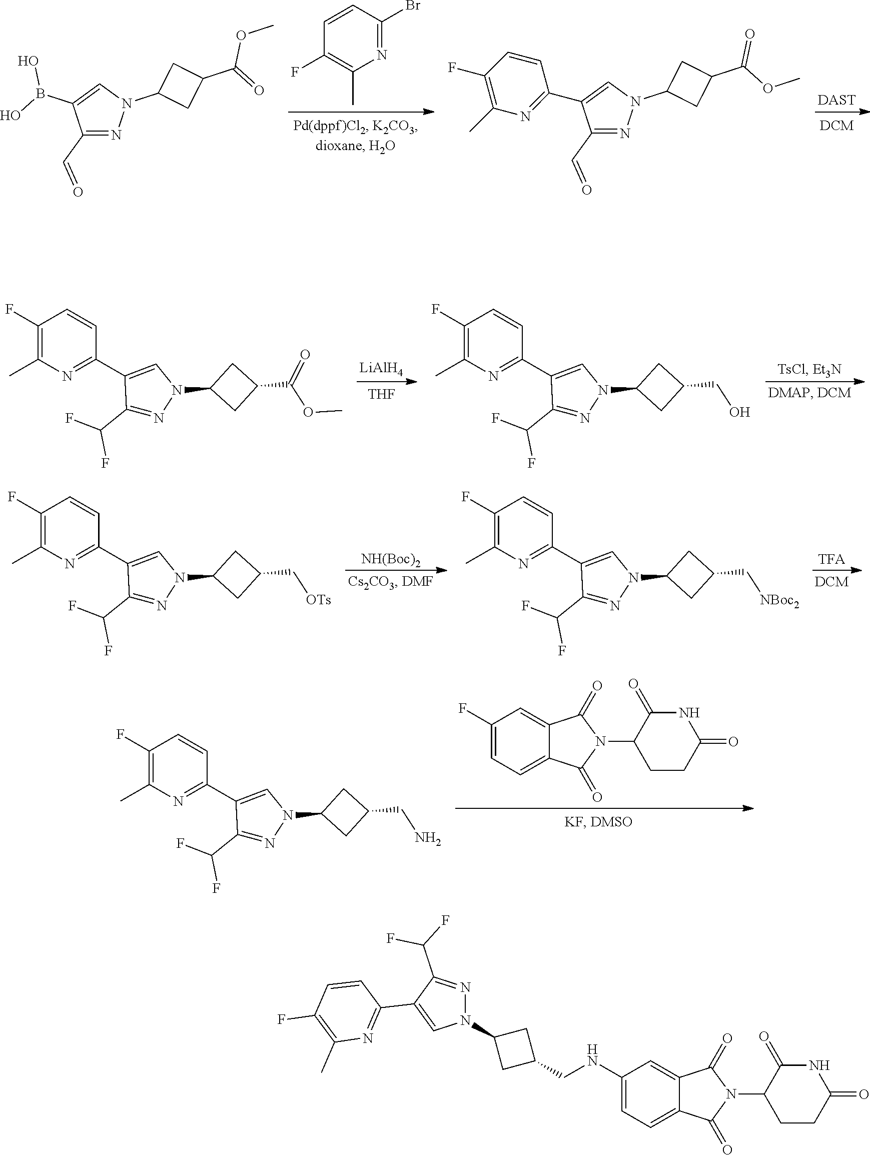

GS-635 (12 mg, 11% yield over 7 steps) was synthesized as a light-yellow solid following the standard procedures for preparing GS-444. MS (ESI) m/z=567.2 [M+H]⁺.

### Example 622. 1-(trans-3-(((2-(2,6-dioxopiperidin-3-yl)-1,3-dioxoisoindolin-5-yl)amino)methyl)cyclobutyl)-N,N-dimethyl-4-(6-methylpyridin-2-yl)-1H-pyrazole-3-carboxamide (GS-636)

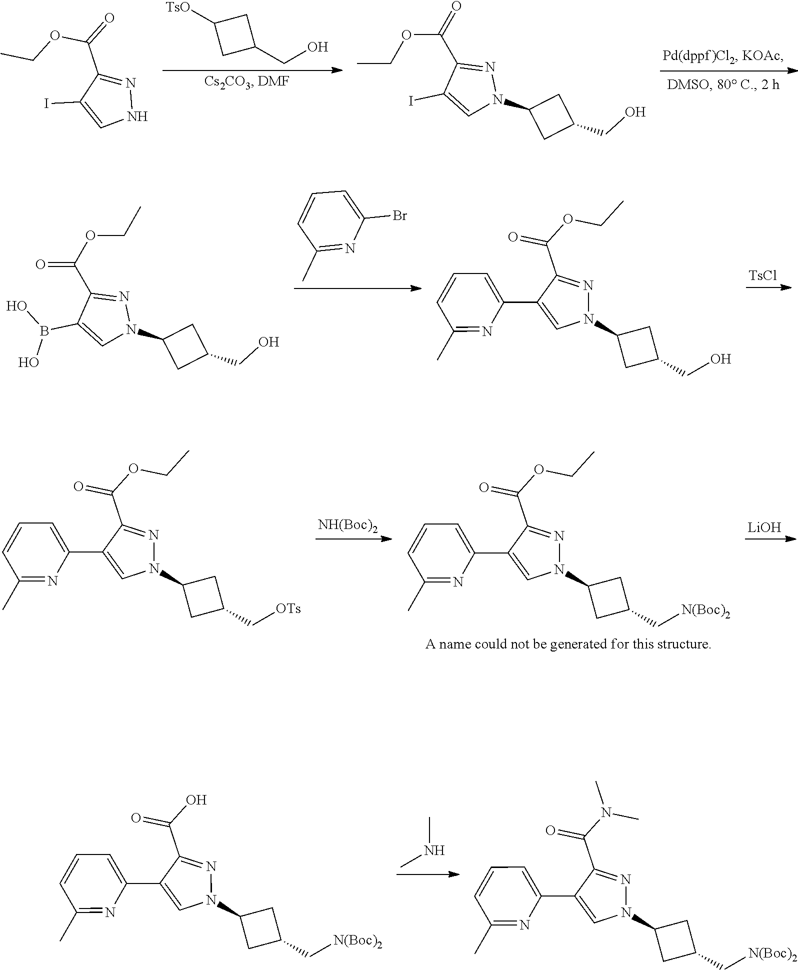

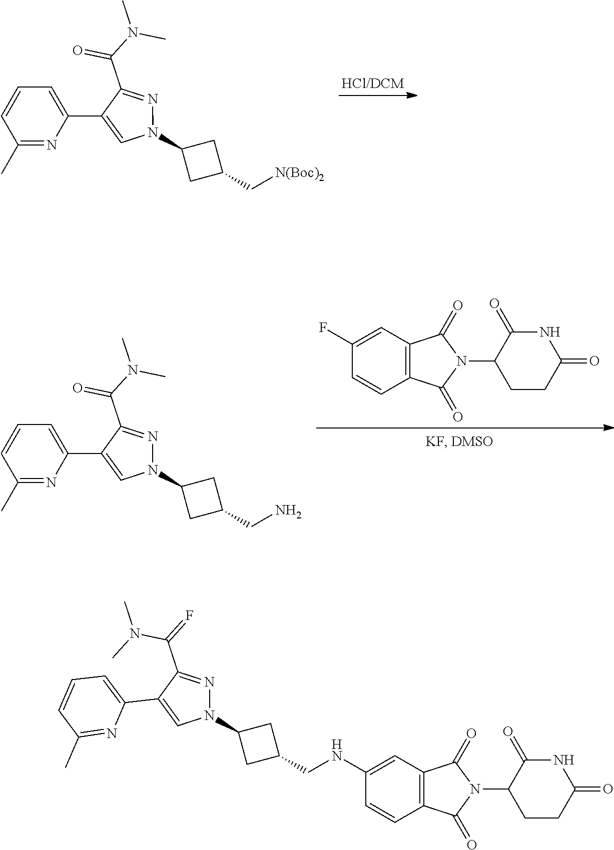

Step 1. Synthesis of ethyl 1-(trans-3-(hydroxymethyl)cyclobutyl)-4-iodo-1H-pyrazole-3-carboxylate

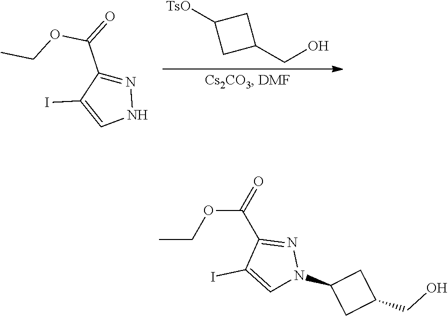

To a solution of ethyl 4-iodo-1H-pyrazole-3-carboxylate (synthesized by a similar procedure for step 1 of GS-568, 2 g, 7.52 mmol) in DMSO (10 mL) were added 3-(hydroxymethyl)cyclobutyl 4-methylbenzenesulfonate (2.89 g, 11.28 mmol) and Cs₂CO₃ (4.89 g, 15.04 mmol). After the mixture was stirred at 90° C. for 16 h, it was purified by reverse phase chromatography (water:MeOH=1:1) to provide the title compound (700 mg, yield: 27%) as yellow oil. MS (ESI) m/z=351.2 [M+H]⁺.

Step 2. Synthesis of (3-(ethoxycarbonyl)-1-(trans-3-(hydroxymethyl)cyclobutyl)-1H-pyrazol-4-yl)boronic acid

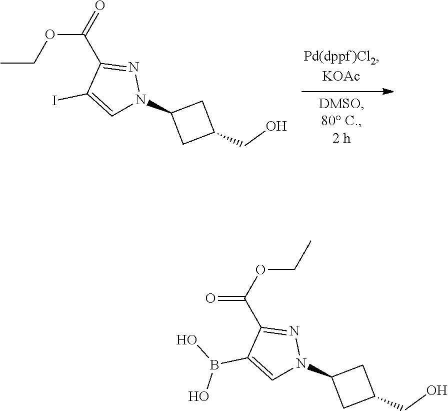 [SMILES: CCOC(=O)c1nn([C@H]2C[C@H](CO)C2)cc1I ; CCOC(=O)c1nn([C@H]2C[C@H](CO)C2)cc1B(O)O]

To a solution of ethyl 1-(trans-3-(hydroxymethyl)cyclobutyl)-4-iodo-1H-pyrazole-3-carboxylate (700 mg, 2.00 mmol) in DMSO (10 mL) were added 4,4,5,5-tetramethyl-2-(4,4,5,5-tetramethyl-1,3,2-dioxaborolan-2-yl)-1,3,2-dioxaborolane (761.48 mg, 3.00 mmol), Pd(dppf)Cl₂ (146.14 mg, 199.91 μmol) and KOAc (391.83 mg, 4.00 mmol). After the mixture was stirred at 80° C. for 2 h, it was purified by reverse phase chromatography (0.1% TFA in water:MeOH=1:1) to provide the title compound (400 mg, yield: 75%) as yellowish solid. MS (ESI) m/z=269.1 [M+H]⁺.

Step 3. Synthesis of ethyl 1-(trans-3-(hydroxymethyl)cyclobutyl)-4-(6-methylpyridin-2-yl)-1H-pyrazole-3-carboxylate

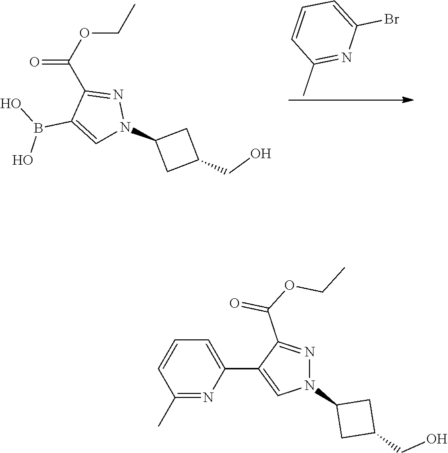

To a solution of (3-(ethoxycarbonyl)-1-(trans-3-(hydroxymethyl)cyclobutyl)-1H-pyrazol-4-yl)boronic acid (400 mg, 1.49 mmol) in water (2 mL) were added 2-bromo-6-methyl-pyridine (282.35 mg, 1.64 mmol), Pd(dppf)Cl₂ (109.07 mg, 149.21 μmol), K₂CO₃ (411.83 mg, 2.98 mmol) and 1,4-dioxane (10 mL). The mixture was stirred at 100° C. for 3 h, before it was concentrated. The resulting residue was purified by reverse phase chromatography (0.1% TFA in water:MeOH=1:1) to provide the title compound (230 mg, yield: 49%) as yellow oil. MS (ESI) m/z=316.3 [M+H]⁺.

Step 4. Synthesis of ethyl 4-(6-methylpyridin-2-yl)-1-(trans-3-((tosyloxy)methyl)cyclobutyl)-1H-pyrazole-3-carboxylate

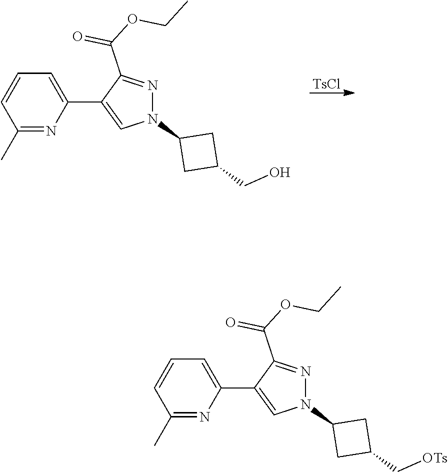 [SMILES: CCOC(=O)c1nn([C@H]2C[C@H](CO)C2)cc1-c1cccc(C)n1 ; "TsCl" ; CCOC(=O)c1nn([C@H]2C[C@H](COS(=O)(=O)c3ccc(C)cc3)C2)cc1-c1cccc(C)n1]

To a solution of ethyl 1-(trans-3-(hydroxymethyl)cyclobutyl)-4-(6-methylpyridin-2-yl)-1H-pyrazole-3-carboxylate (230 mg, 729.31 μmol) in DCM (10 mL) were added TsCl (77.16 mg, 1.09 mmol), TEA (147.60 mg, 1.46 mmol) and DMAP (8.91 mg, 72.93 μmol). The mixture was stirred at rt for 16 h, before it was concentrated. The resulting residue was purified by flash chromatography (petroleum ether:EtOAc=1:1) to provide the title compound (230 mg, yield: 67%) as yellow oil. MS (ESI) m/z=470.3 [M+H]⁺.

Step 5. Synthesis of ethyl 1-[3-[[bis(tert-butoxycarbonyl)amino]methyl]cyclobutyl]-4-(6-methyl-2-pyridyl)pyrazole-3-carboxylate

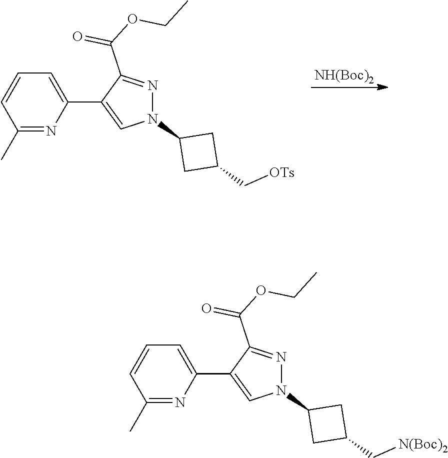

To a solution of ethyl 4-(6-methylpyridin-2-yl)-1-(trans-3-((tosyloxy)methyl)cyclobutyl)-1H-pyrazole-3-carboxylate (230 mg, 489.83 μmol) in DMF (5 mL) were added di-tert-butyl iminodicarboxylate (127.70 mg, 587.79 μmol) and Cs₂CO₃ (318.39 mg, 979.66 μmol). After the mixture was stirred at 90° C. for 1 h, it was purified by reverse phase chromatography (0.1% TFA in water:MeOH=1:1) to provide the title compound (230 mg, yield: 91%) as yellow oil. MS (ESI) m/z=515.4 [M+H]⁺.

Step 6. Synthesis of 1-[3-[[bis(tert-butoxycarbonyl)amino]methyl]cyclobutyl]-4-(6-methyl-2-pyridyl)pyrazole-3-carboxylic acid

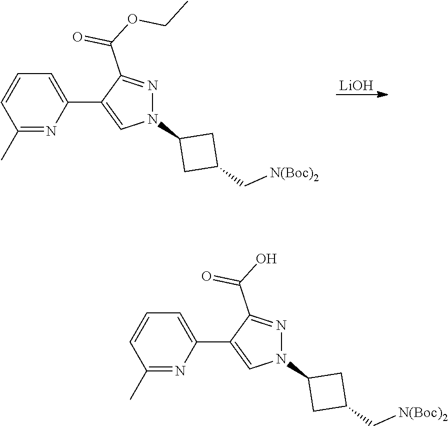

To a solution of ethyl 1-[3-[[bis(tert-butoxycarbonyl)amino]methyl]cyclobutyl]-4-(6-methyl-2-pyridyl)pyrazole-3-carboxylate (230 mg, 446.94 μmol) in water (1 mL) were added LiOH (10.70 mg, 446.94 μmol) and THF (1 mL). After the mixture was stirred at rt for 2 h, it was purified by reverse phase chromatography (water:MeOH=1:1) to provide the title compound (200 mg, yield: 92%) as yellow oil. MS (ESI) m/z=487.4.4 [M+H]⁺.

Step 7. Synthesis of tert-butyl N-tert-butoxycarbonyl-N-[[3-[3-(dimethylcarbamoyl)-4-(6-methyl-2-pyridyl)pyrazol-1-yl]cyclobutyl]methyl]carbamate

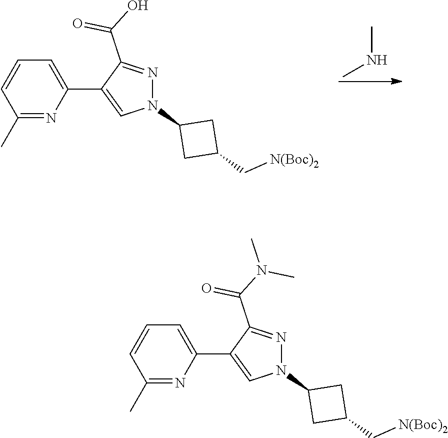

To a solution of 1-[3-[[bis(tert-butoxycarbonyl)amino]methyl]cyclobutyl]-4-(6-methyl-2-pyridyl)pyrazole-3-carboxylic acid (100 mg, 205.52 μmol) in DMF (2 mL) were added dimethylamine (2 M in THF, 0.2 mL), HATU (117.15 mg, 308.29 μmol) and DIPEA (79.69 mg, 616.57 μmol, 101.90 uL). After the mixture was stirred at rt for 2 h, it was purified by reverse phase chromatography (water:MeOH=1:1) to provide the title compound (70 mg, yield: 66%) as yellowish oil. MS (ESI) m/z=514.4 [M+H]⁺.

Step 8. Synthesis of 1-(trans-3-(aminomethyl)cyclobutyl)-N,N-dimethyl-4-(6-methylpyridin-2-yl)-1H-pyrazole-3-carboxamide

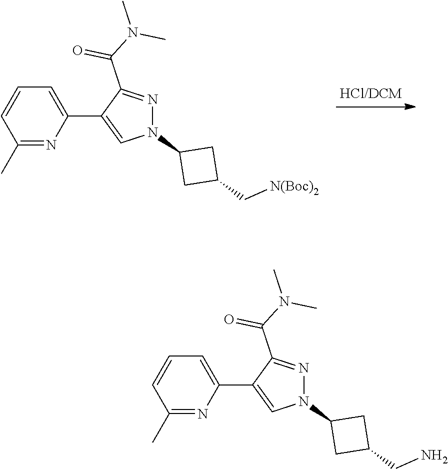

To a solution of tert-butyl N-tert-butoxycarbonyl-N-[[3-[3-(dimethylcarbamoyl)-4-(6-methyl-2-pyridyl)pyrazol-1-yl]cyclobutyl]methyl]carbamate (70 mg, 136.29 μmol) in DCM (1 mL) was added HCl solution (4 M in dioxane, 1 mL). The mixture was stirred at rt for 1 h, before it was concentrated to provide the title compound (40 mg, yield: 94%) as white solid. MS (ESI) m/z=314.4 [M+H]⁺.

Step 9. Synthesis of 1-(trans-3-(((2-(2,6-dioxopiperidin-3-yl)-1,3-dioxoisoindolin-5-yl)amino)methyl)cyclobutyl)-N,N-dimethyl-4-(6-methylpyridin-2-yl)-1H-pyrazole-3-carboxamide

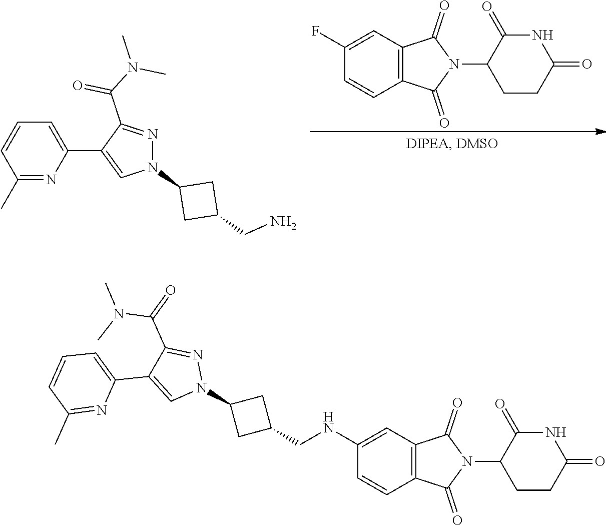 [SMILES: Cc1cccc(-c2cn([C@H]3C[C@H](CN)C3)nc2C(=O)N(C)C)n1 ; O=C1CCC(N2C(=O)c3ccc(F)cc3C2=O)C(=O)N1 ; Cc1cccc(-c2cn([C@H]3C[C@H](CNc4ccc5c(c4)C(=O)N(C4CCC(=O)NC4=O)C5=O)C3)nc2C(=O)N(C)C)n1]

To a solution of 1-(trans-3-(aminomethyl)cyclobutyl)-N,N-dimethyl-4-(6-methylpyridin-2-yl)-1H-pyrazole-3-carboxamide (40 mg, 127.63 μmol) in DMSO (1 mL) were added 2-(2,6-dioxo-3-piperidyl)-5-fluoro-isoindoline-1,3-dione (42.31 mg, 153.16 μmol) and DIPEA (32.99 mg, 255.27 μmol, 42.19 uL). After the mixture was stirred at 130° C. for 3 h, it was purified by reverse phase chromatography (water:MeOH=1:1) to provide the title compound (15 mg, yield: 21%) as yellow solid. MS (ESI) m/z=570.4 [M+H]⁺.

### Example 623. 1-(trans-3-(((2-(2,6-dioxopiperidin-3-yl)-1,3-dioxoisoindolin-5-yl)amino)methyl)cyclobutyl)-N-methyl-4-(6-methylpyridin-2-yl)-1H-pyrazole-3-carboxamide (GS-637)

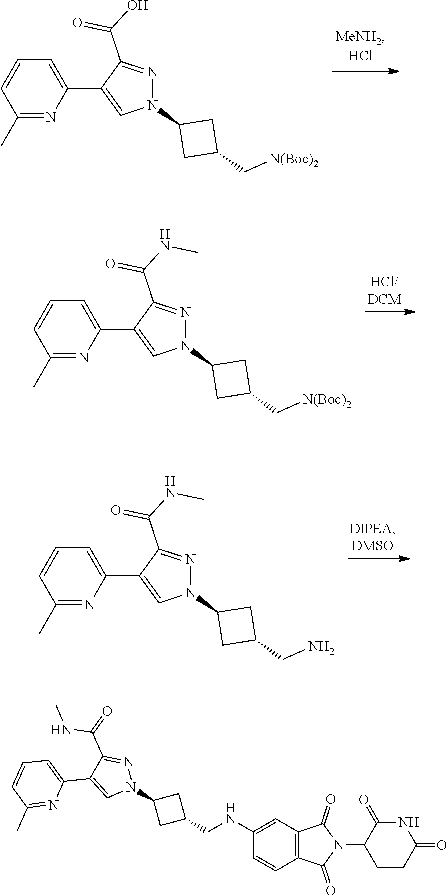

GS-637 was synthesized following the similar procedures as described for GS-636 (15 mg, yield over 3 steps: 13%) as yellow solid. MS (ESI) m/z=556.3 [M+H]⁺.

### Example 624. 2-(2,6-Dioxopiperidin-3-yl)-5-(((trans-3-(4-(6-methylpyridin-2-yl)-3-(piperidin-4-yl)-1H-pyrazol-1-yl)cyclobutyl)methyl)amino)isoindoline-1,3-dione (GS-638)

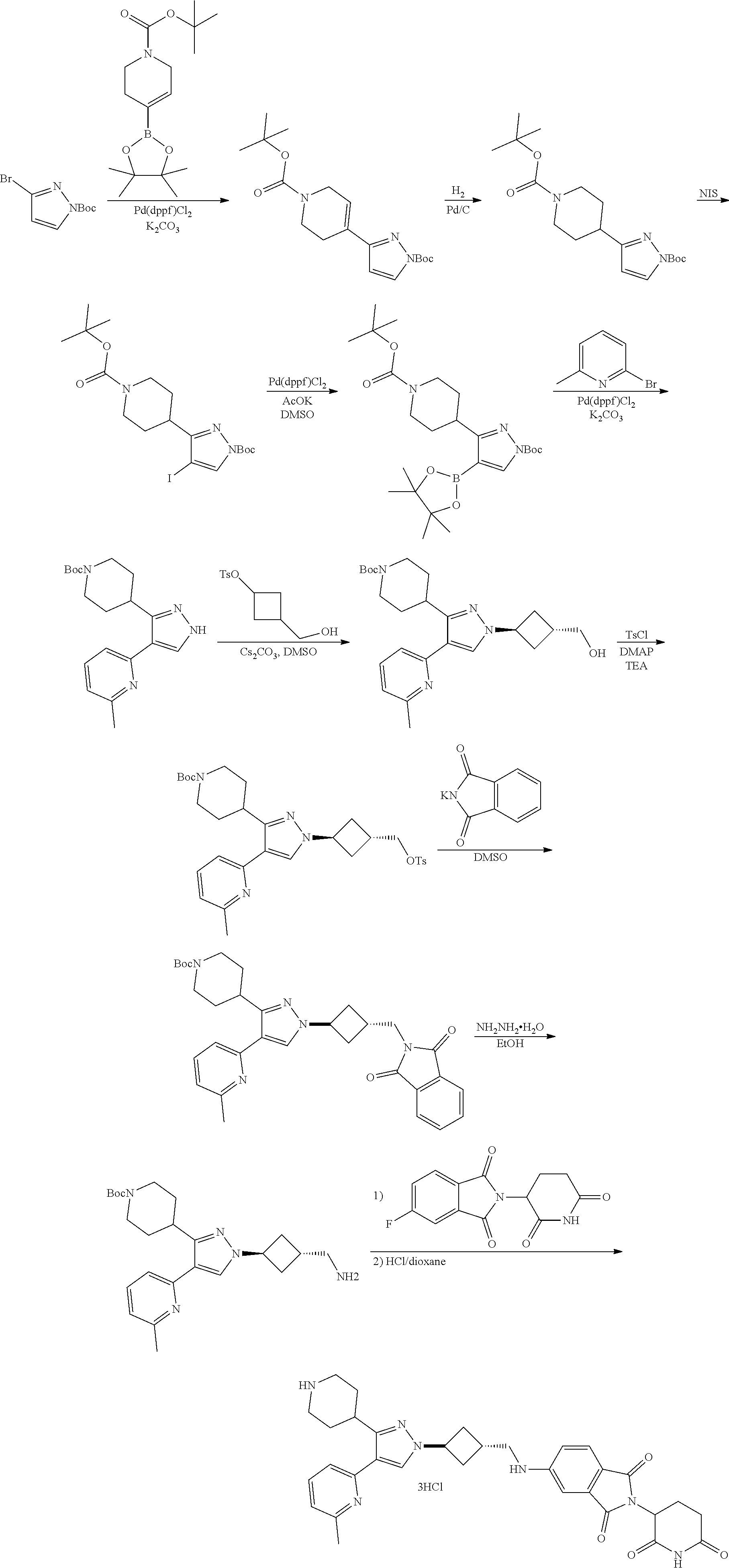

Steps 1 to 5. Synthesis of tert-butyl 4-(4-(6-methylpyridin-2-yl)-1H-pyrazol-3-yl)piperidine-1-carboxylate

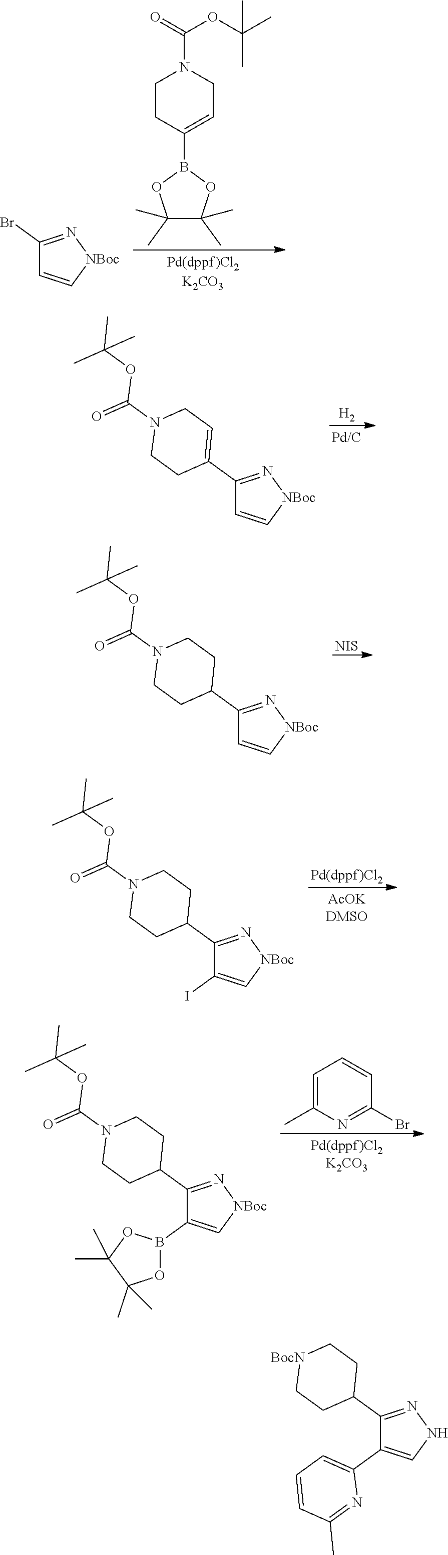

The title compound was synthesized following the similar procedures as described for steps 1 to 5 of GS-071 (167 mg, yield over 5 steps: 7%) as yellow solid. MS (ESI) m/z=587.7 [M+H]⁺.

Steps 6 to 7. Synthesis of tert-butyl 4-(4-(6-methylpyridin-2-yl)-1-(trans-3-((tosyloxy)methyl)cyclobutyl)-1H-pyrazol-3-yl)piperidine-1-carboxylate

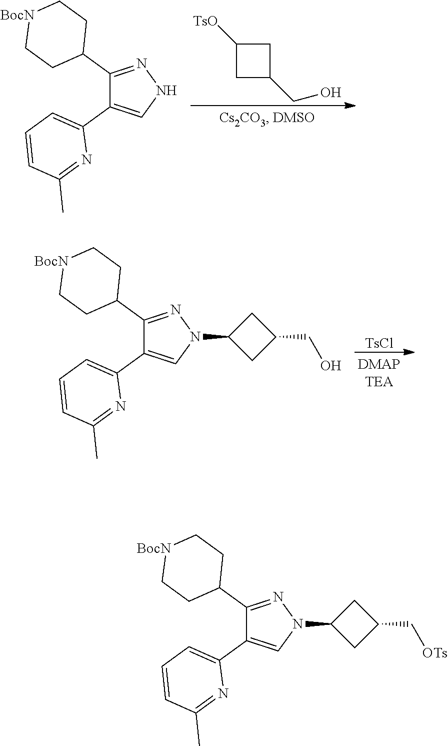

The title compound was synthesized following the similar procedures as described or steps 3 to 4 of GS-579 (60 mg, yield over 2 steps: 6%) as light brown oil. MS (ESI) m/z=581.5 [M+H]⁺.

Step 8. Synthesis of tert-butyl 4-(1-(trans-3-((1,3-dioxoisoindolin-2-yl)methyl)cyclobutyl)-4-(6-methylpyridin-2-yl)-1H-pyrazol-3-yl)piperidine-1-carboxylate

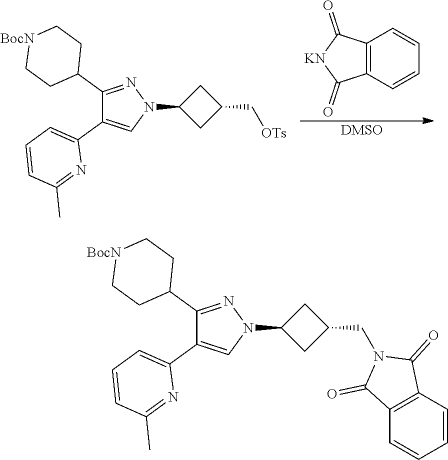

A mixture of tert-butyl 4-(4-(6-methylpyridin-2-yl)-1-(trans-3-((tosyloxy)methyl)cyclobutyl)-1H-pyrazol-3-yl)piperidine-1-carboxylate (60 mg, 53.72 μmol) and (1,3-dioxoisoindolin-2-yl)potassium (30 mg, 161.97 μmol) in DMSO (3 mL) was heated at 90° C. for 1 h. The mixture was cooled to rt and purified by reverse phase chromatography to provide the title compound (30 mg, yield: 76%) as white solid. MS (ESI) m/z=556.4 [M+H]⁺.

Step 9. Synthesis of tert-butyl 4-(1-(trans-3-(aminomethyl)cyclobutyl)-4-(6-methylpyridin-2-yl)-1H-pyrazol-3-yl)piperidine-1-carboxylate

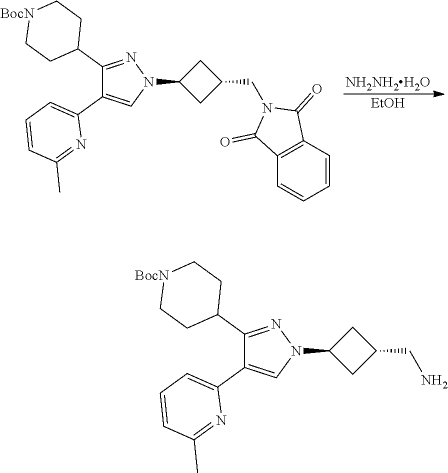

To a solution of tert-butyl 4-(1-(trans-3-((1,3-dioxoisoindolin-2-yl)methyl)cyclobutyl)-4-(6-methylpyridin-2-yl)-1H-pyrazol-3-yl)piperidine-1-carboxylate (30 mg, 41.03 μmol) in ethanol (3 mL) was added NH₂NH₂·H₂O (0.1 mL) at rt. The reaction mixture was heated at reflux for 2 h, before it was concentrated to provide the title compound (30 mg, yield: 70%) as white solid. MS (ESI) m/z=426.5 [M+H]⁺.

Step 10. Synthesis of 2-(2,6-dioxopiperidin-3-yl)-5-(((trans-3-(4-(6-methylpyridin-2-yl)-3-(piperidin-4-yl)-1H-pyrazol-1-yl)cyclobutyl)methyl)amino)isoindoline-1,3-dione

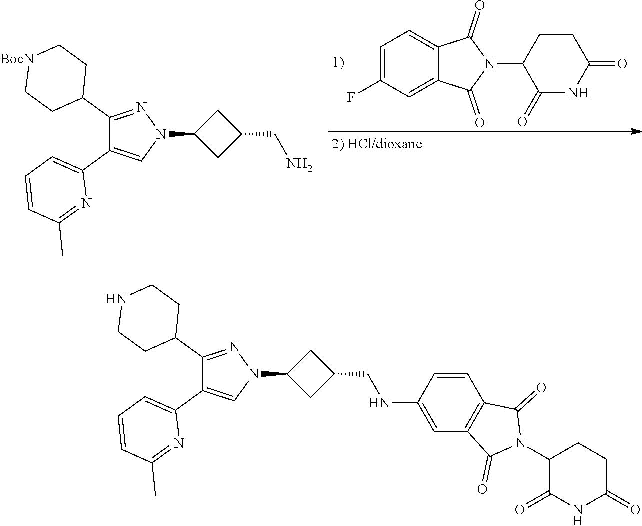 [SMILES: Cc1cccc(-c2cn([C@H]3C[C@H](CN)C3)nc2C2CCN(C(=O)OC(C)(C)C)CC2)n1 ; O=C1CCC(N2C(=O)c3ccc(F)cc3C2=O)C(=O)N1 ; Cc1cccc(-c2cn([C@H]3C[C@H](CNc4ccc5c(c4)C(=O)N(C4CCC(=O)NC4=O)C5=O)C3)nc2C2CCNCC2)n1]

A mixture of tert-butyl 4-(1-(trans-3-(aminomethyl)cyclobutyl)-4-(6-methylpyridin-2-yl)-1H-pyrazol-3-yl)piperidine-1-carboxylate (30 mg, 28.90 μmol), 2-(2,6-dioxo-3-piperidyl)-5-fluoro-isoindoline-1,3-dione (20 mg, 72.41 μmol) and KF (6 mg, 103.28 μmol) in DMSO (1.5 mL) was heated at 125° C. under microwave irradiation for 20 min. After the mixture was cooled to rt, it was purified by reverse phase chromatography to provide the yellow solid, which was dissolved in DCM (2 mL). To the solution was added HCl solution (4 M in dioxane, 0.5 mL). The mixture was stirred at rt for 1 h, before it was concentrated. The resulting residue was purified by prep-HPLC to provide the title compound (0.8 mg, yield: 50%) as yellow solid. MS (ESI) m/z=582.7 [M+H]⁺.

### Example 625. 2-(1-Methyl-2,6-dioxopiperidin-3-yl)-5-((3-(trans-3-(3-methyl-4-(quinoxalin-2-yl)-1H-pyrazol-1-yl)cyclobutyl)propyl)amino)isoindoline-1,3-dione (GS-639)

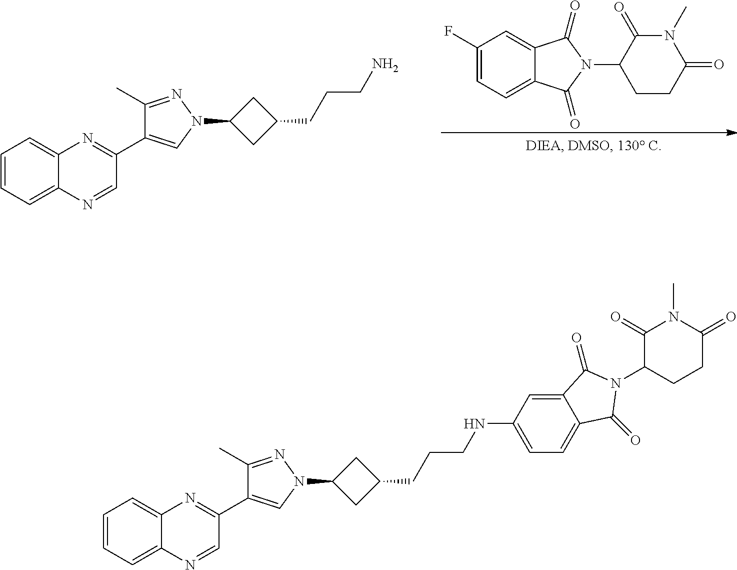 [SMILES: Cc1nn([C@H]2C[C@H](CCCN)C2)cc1-c1cnc2ccccc2n1 ; CN1C(=O)CCC(N2C(=O)c3ccc(F)cc3C2=O)C1=O ; Cc1nn([C@H]2C[C@H](CCCNc3ccc4c(c3)C(=O)N(C3CCC(=O)N(C)C3=O)C4=O)C2)cc1-c1cnc2ccccc2n1]

To a solution of 3-(trans-3-(3-methyl-4-(quinoxalin-2-yl)-1H-pyrazol-1-yl)cyclobutyl)propan-1-amine (221 mg, 687.58 μmol) in DMoX (3 mL) were added 5-fluoro-2-(2-methyl-2,6-dioxo-3-piperidyl)isoindoline-1,3-dione (199.57 mg, 687.58 μmol) and DJEA (264.03 mg, 2.06 mmol). The mixture was irradiated at 130° C. in the microwave reactor for 30 min. Then the mixture was purified by reverse phase chromatography to provide the title compound (98 mg, yield: 240%) as yellow solid. (MIS (ESI) m/z=592.7 [M+H]⁺.

Certain compounds disclosed herein have the structures shown in Table 1.

TABLE 1

| Cpd. code | Structure | Chemical Name |
|---|---|---|
| GS-001 | 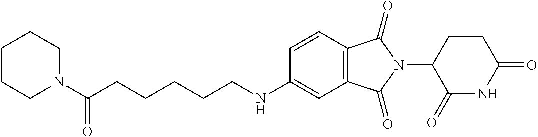 [SMILES: O=C1CCC(N2C(=O)c3ccc(NCCCCCC(=O)N4CCCCC4)cc3C2=O)C(=O)N1] | 2-(2,6-dioxopiperidin-3-yl)-5-((6-oxo-6- (piperidin-1-yl)hexyl)amino)isoindoline- 1,3-dione |
| GS-002 | 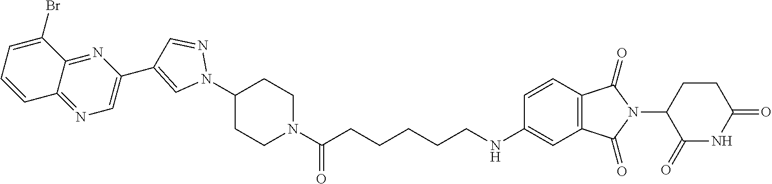 [SMILES: O=C1CCC(N2C(=O)c3ccc(NCCCCCC(=O)N4CCC(n5cc(-c6cnc7cccc(Br)c7n6)cn5)CC4)cc3C2=O)C(=O)N1] | 5-((6-(4-(4-(8-bromoquinoxalin-2-yl)- 1H-pyrazol-1-yl)piperidin-1-yl)-6- oxohexyl)amino)-2-(2,6-dioxopiperidin- 3-yl)isoindoline-1,3-dione |
| GS-003 | 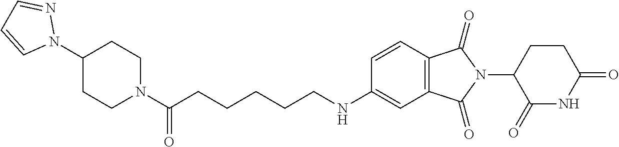 [SMILES: O=C1CCC(N2C(=O)c3ccc(NCCCCCC(=O)N4CCC(n5cccn5)CC4)cc3C2=O)C(=O)N1] | 5-((6-(4-(1H-pyrazol-1-yl)piperidin-1- yl)-6-oxohexyl)amino)-2-(2,6- dioxopiperidin-3-yl)isoindoline-1,3-dione |
| GS-004 | 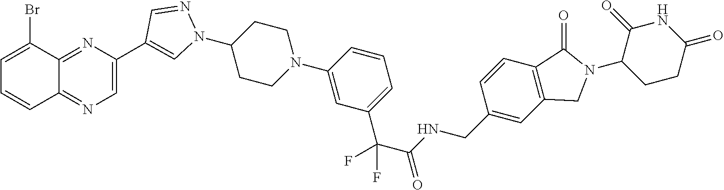 [SMILES: O=C1CCC(N2Cc3cc(CNC(=O)C(F)(F)c4cccc(N5CCC(n6cc(-c7cnc8cccc(Br)c8n7)cn6)CC5)c4)ccc3C2=O)C(=O)N1] | 2-(3-(4-(4-(8-bromoquinoxalin-2-yl)-1H- pyrazol-1-yl)piperidin-1-yl)phenyl)-N- ((2-(2,6-dioxopiperidin-3-yl)-1- oxoisoindolin-5-yl)methyl)-2,2- difluoroacetamide |
| GS-005 | 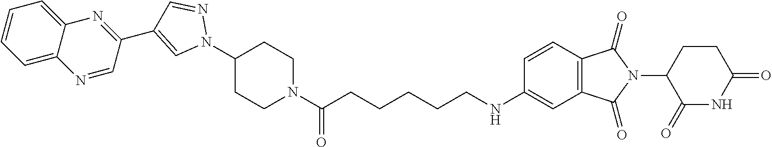 [SMILES: O=C1CCC(N2C(=O)c3ccc(NCCCCCC(=O)N4CCC(n5cc(-c6cnc7ccccc7n6)cn5)CC4)cc3C2=O)C(=O)N1] | 2-(2,6-dioxopiperidin-3-yl)-5-((6-oxo-6- (4-(4-(quinoxalin-2-yl)-1H-pyrazol-1- yl)piperidin-1- yl)hexyl)amino)isoindoline-1,3-dione |
| GS-006 | 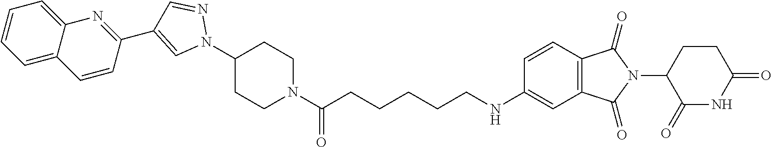 [SMILES: O=C1CCC(N2C(=O)c3ccc(NCCCCCC(=O)N4CCC(n5cc(-c6ccc7ccccc7n6)cn5)CC4)cc3C2=O)C(=O)N1] | 2-(2,6-dioxopiperidin-3-yl)-5-((6-oxo-6- (4-(4-(quinolin-2-yl)-1H-pyrazol-1- yl)piperidin-1- yl)hexyl)amino)isoindoline-1,3-dione |
| GS-007 | 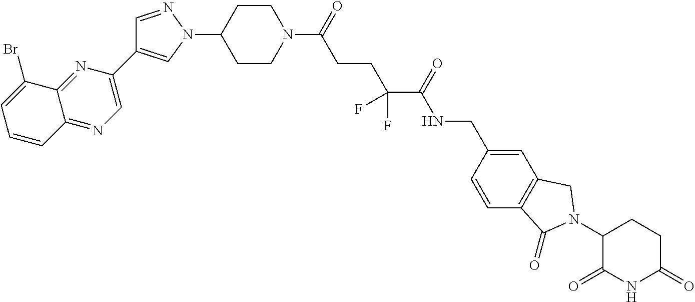 [SMILES: O=C1CCC(N2Cc3cc(CNC(=O)C(F)(F)CCC(=O)N4CCC(n5cc(-c6cnc7cccc(Br)c7n6)cn5)CC4)ccc3C2=O)C(=O)N1] | 5-(4-(4-(8-bromoquinoxalin-2-yl)-1H- pyrazol-1-yl)piperidin-1-yl)-N-((2-(2,6- dioxopiperidin-3-yl)-1-oxoisoindolin-5- yl)methyl)-2,2-difluoro-5- oxopentanamide |
| GS-008 | 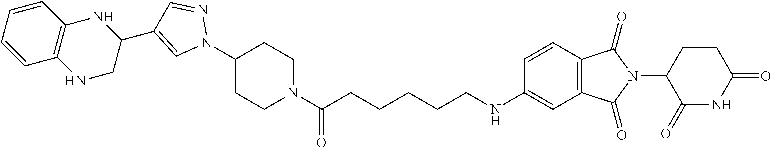 [SMILES: O=C1CCC(N2C(=O)c3ccc(NCCCCCC(=O)N4CCC(n5cc(C6CNc7ccccc7N6)cn5)CC4)cc3C2=O)C(=O)N1] | 2-(2,6-dioxopiperidin-3-yl)-5-((6-oxo-6- (4-(4-(1,2,3,4-tetrahydroquinoxalin-2-yl)- 1H-pyrazol-1-yl)piperidin-1- yl)hexyl)amino)isoindoline-1,3-dione |
| GS-009 | 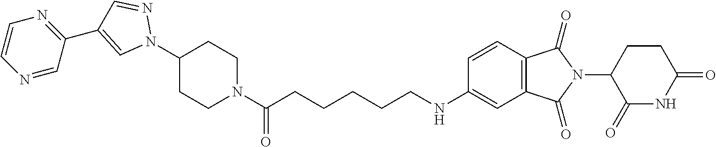 [SMILES: O=C1CCC(N2C(=O)c3ccc(NCCCCCC(=O)N4CCC(n5cc(-c6cnccn6)cn5)CC4)cc3C2=O)C(=O)N1] | 2-(2,6-dioxopiperidin-3-yl)-5-((6-oxo-6- (4-(4-(pyrazin-2-yl)-1H-pyrazol-1- yl)piperidin-1- yl)hexyl)amino)isoindoline-1,3-dione |
| GS-010 | 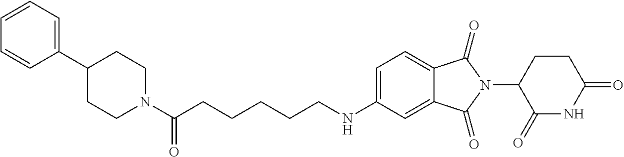 [SMILES: O=C1CCC(N2C(=O)c3ccc(NCCCCCC(=O)N4CCC(c5ccccc5)CC4)cc3C2=O)C(=O)N1] | 2-(2,6-dioxopiperidin-3-yl)-5-((6-oxo-6- (4-phenylpiperidin-1- yl)hexyl)amino)isoindoline-1,3-dione |
| GS-011 | 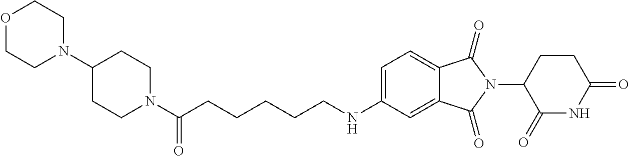 [SMILES: O=C1CCC(N2C(=O)c3ccc(NCCCCCC(=O)N4CCC(N5CCOCC5)CC4)cc3C2=O)C(=O)N1] | 2-(2,6-dioxopiperidin-3-yl)-5-((6-(4- morpholinopiperidin-1-yl)-6- oxohexyl)amino)isoindoline-1,3-dione |
| GS-012 | 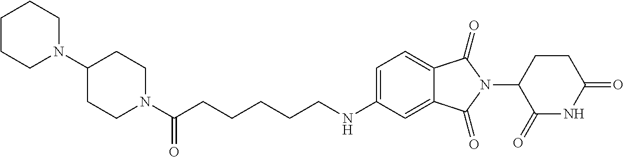 [SMILES: O=C1CCC(N2C(=O)c3ccc(NCCCCCC(=O)N4CCC(N5CCCCC5)CC4)cc3C2=O)C(=O)N1] | 5-((6-([1,4′-bipiperidin]-1′-yl)-6- oxohexyl)amino)-2-(2,6-dioxopiperidin- 3-yl)isoindoline-1,3-dione |
| GS-013 | 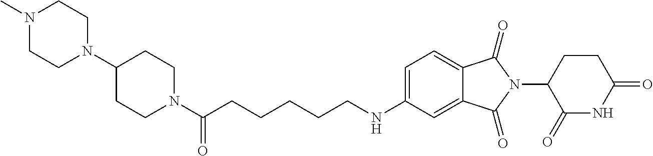 [SMILES: CN1CCN(C2CCN(C(=O)CCCCCNc3ccc4c(c3)C(=O)N(C3CCC(=O)NC3=O)C4=O)CC2)CC1] | 2-(2,6-dioxopiperidin-3-yl)-5-((6-(4-(4- methylpiperazin-1-yl)piperidin-1-yl)-6- oxohexyl)amino)isoindoline-1,3-dione |
| GS-014 | 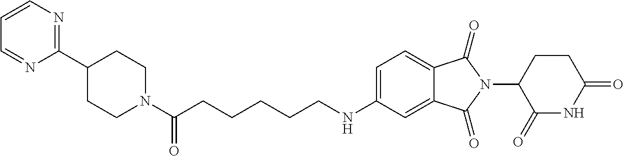 [SMILES: O=C1CCC(N2C(=O)c3ccc(NCCCCCC(=O)N4CCC(c5ncccn5)CC4)cc3C2=O)C(=O)N1] | 2-(2,6-dioxopiperidin-3-yl)-5-((6-oxo-6- (4-(pyrimidin-2-yl)piperidin-1- yl)hexyl)amino)isoindoline-1,3-dione |
| GS-015 | 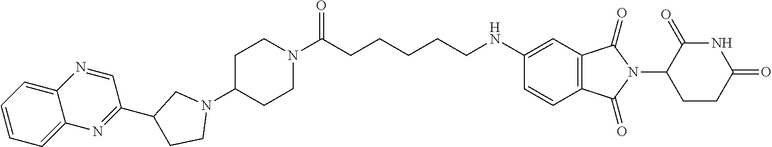 [SMILES: O=C1CCC(N2C(=O)c3ccc(NCCCCCC(=O)N4CCC(N5CCC(c6cnc7ccccc7n6)C5)CC4)cc3C2=O)C(=O)N1] | 2-(2,6-dioxopiperidin-3-yl)-5-((6-oxo-6- (4-(3-(quinoxalin-2-yl)pyrrolidin-1- yl)piperidin-1- yl)hexyl)amino)isoindoline-1,3-dione |
| GS-016 | 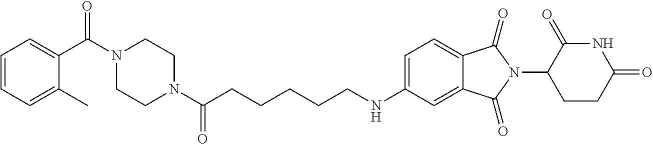 [SMILES: Cc1ccccc1C(=O)N1CCN(C(=O)CCCCCNc2ccc3c(c2)C(=O)N(C2CCC(=O)NC2=O)C3=O)CC1] | 2-(2,6-dioxopiperidin-3-yl)-5-((6-(4-(2- methylbenzoyl)piperazin-1-yl)-6- oxohexyl)amino)isoindoline-1,3-dione |
| GS-017 | 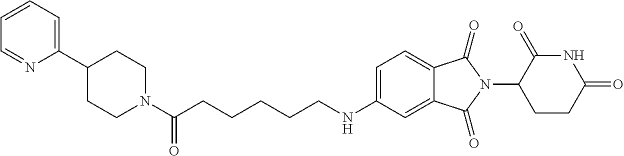 [SMILES: O=C1CCC(N2C(=O)c3ccc(NCCCCCC(=O)N4CCC(c5ccccn5)CC4)cc3C2=O)C(=O)N1] | 2-(2,6-dioxopiperidin-3-yl)-5-((6-oxo-6- (4-(pyridin-2-yl)piperidin-1- yl)hexyl)amino)isoindoline-1,3-dione |
| GS-018 | 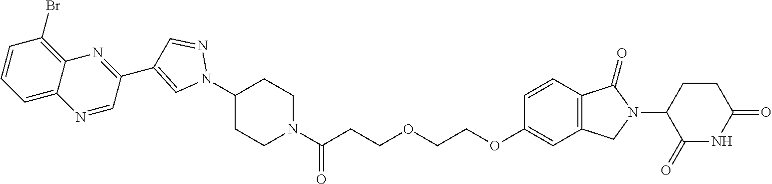 [SMILES: O=C1CCC(N2Cc3cc(OCCOCCC(=O)N4CCC(n5cc(-c6cnc7cccc(Br)c7n6)cn5)CC4)ccc3C2=O)C(=O)N1] | 3-(5-(2-(3-(4-(4-(8-bromoquinoxalin-2- yl)-1H-pyrazol-1-yl)piperidin-1-yl)-3- oxopropoxy)ethoxy)-1-oxoisoindolin-2- yl)piperidine-2,6-dione |
| GS-019 | 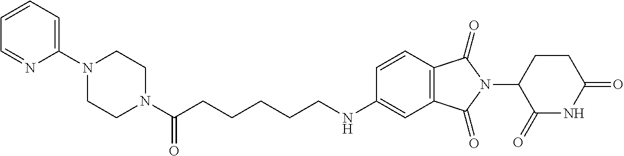 [SMILES: O=C1CCC(N2C(=O)c3ccc(NCCCCCC(=O)N4CCN(c5ccccn5)CC4)cc3C2=O)C(=O)N1] | 2-(2,6-dioxopiperidin-3-yl)-5-((6-oxo-6- (4-(pyridin-2-yl)piperazin-1- yl)hexyl)amino)isoindoline-1,3-dione |
| GS-020 | 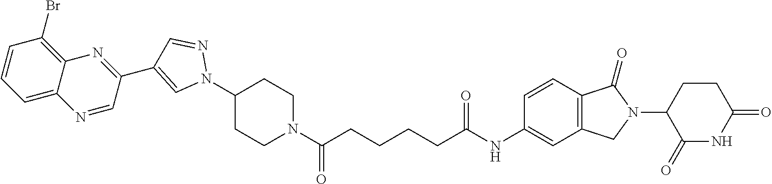 [SMILES: O=C1CCC(N2Cc3cc(NC(=O)CCCCC(=O)N4CCC(n5cc(-c6cnc7cccc(Br)c7n6)cn5)CC4)ccc3C2=O)C(=O)N1] | 6-(4-(4-(8-bromoquinoxalin-2-yl)-1H- pyrazol-1-yl)piperidin-1-yl)-N-(2-(2,6- dioxopiperidin-3-yl)-1-oxoisoindolin-5- yl)-6-oxohexanamide |
| GS-021 | 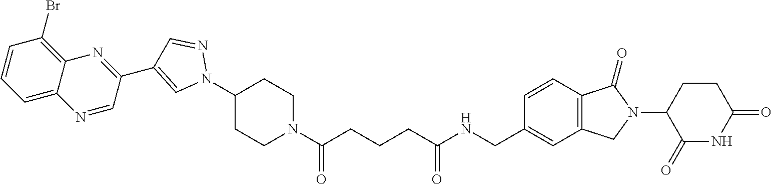 [SMILES: O=C(CCCC(=O)N1CCC(n2cc(-c3cnc4cccc(Br)c4n3)cn2)CC1)NCc1ccc2c(c1)CN(C1CCC(=O)NC1=O)C2=O] | 5-(4-(4-(8-bromoquinoxalin-2-yl)-1H- pyrazol-1-yl)piperidin-1-yl)-N-((2-(2,6- dioxopiperidin-3-yl)-1-oxoisoindolin-5- yl)methyl)-5-oxopentanamide |
| GS-022 | 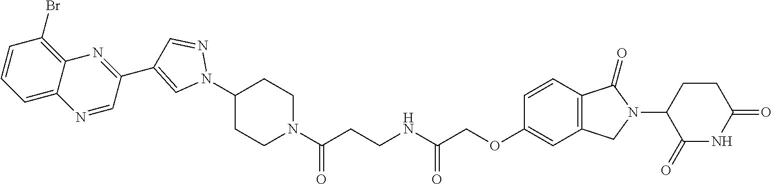 [SMILES: O=C(COc1ccc2c(c1)CN(C1CCC(=O)NC1=O)C2=O)NCCC(=O)N1CCC(n2cc(-c3cnc4cccc(Br)c4n3)cn2)CC1] | N-(3-(4-(4-(8-bromoquinoxalin-2-yl)-1H- pyrazol-1-yl)piperidin-1-yl)-3- oxopropyl)-2-((2-(2,6-dioxopiperidin-3- yl)-1-oxoisoindolin-5-yl)oxy)acetamide |
| GS-023 | 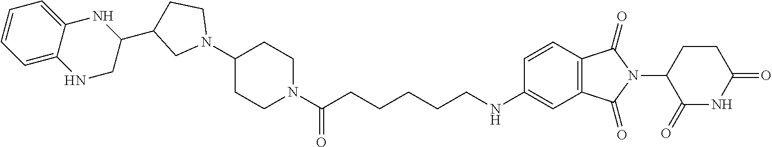 [SMILES: O=C1CCC(N2C(=O)c3ccc(NCCCCCC(=O)N4CCC(N5CCC(C6CNc7ccccc7N6)C5)CC4)cc3C2=O)C(=O)N1] | 2-(2,6-dioxopiperidin-3-yl)-5-((6-oxo-6- (4-(3-(1,2,3,4-tetrahydroquinoxalin-2- yl)pyrrolidin-1-yl)piperidin-1- yl)hexyl)amino)isoindoline-1,3-dione |
| GS-024 | 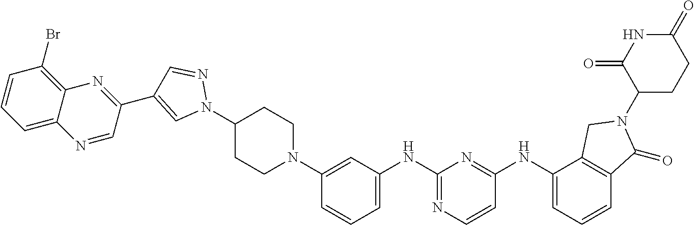 [SMILES: O=C1CCC(N2Cc3c(Nc4ccnc(Nc5cccc(N6CCC(n7cc(-c8cnc9cccc(Br)c9n8)cn7)CC6)c5)n4)cccc3C2=O)C(=O)N1] | 3-(4-((2-((3-(4-(4-(8-bromoquinoxalin-2- yl)-1H-pyrazol-1-yl)piperidin-1- yl)phenyl)amino)pyrimidin-4-yl)amino)- 1-oxoisoindolin-2-yl)piperidine-2,6-dione |
| GS-025 | 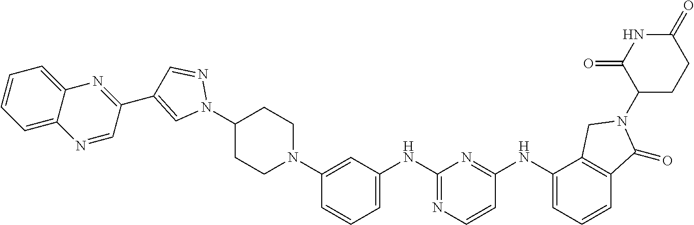 [SMILES: O=C1CCC(N2Cc3c(Nc4ccnc(Nc5cccc(N6CCC(n7cc(-c8cnc9ccccc9n8)cn7)CC6)c5)n4)cccc3C2=O)C(=O)N1] | 3-(1-oxo-4-((2-((3-(4-(4-(quinoxalin-2- yl)-1H-pyrazol-1-yl)piperidin-1- yl)phenyl)amino)pyrimidin-4- yl)amino)isoindolin-2-yl)piperidine-2,6- dione |
| GS-026 | 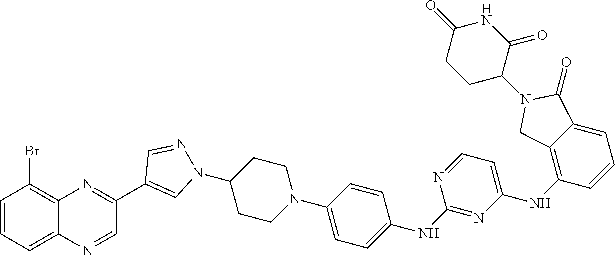 [SMILES: O=C1CCC(N2Cc3c(Nc4ccnc(Nc5ccc(N6CCC(n7cc(-c8cnc9cccc(Br)c9n8)cn7)CC6)cc5)n4)cccc3C2=O)C(=O)N1] | 3-(4-((2-((4-(4-(4-(8-bromoquinoxalin-2- yl)-1H-pyrazol-1-yl)piperidin-1- yl)phenyl)amino)pyrimidin-4-yl)amino)- 1-oxoisoindolin-2-yl)piperidine-2,6-dione |
| GS-027 | 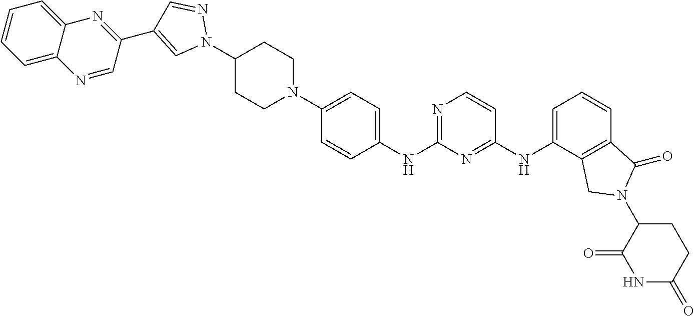 [SMILES: O=C1CCC(N2Cc3c(Nc4ccnc(Nc5ccc(N6CCC(n7cc(-c8cnc9ccccc9n8)cn7)CC6)cc5)n4)cccc3C2=O)C(=O)N1] | 3-(1-oxo-4-((2-((4-(4-(4-(quinoxalin-2- yl)-1H-pyrazol-1-yl)piperidin-1- yl)phenyl)amino)pyrimidin-4- yl)amino)isoindolin-2-yl)piperidine-2,6- dione |
| GS-028 | 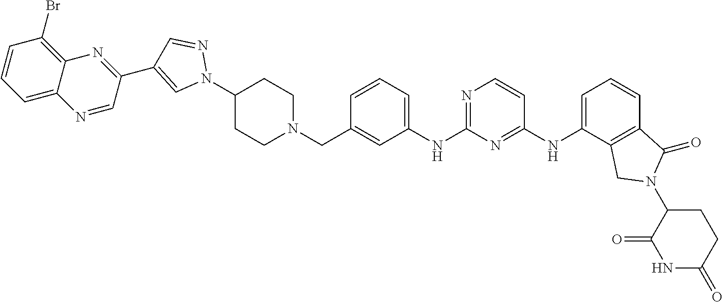 [SMILES: O=C1CCC(N2Cc3c(Nc4ccnc(Nc5cccc(CN6CCC(n7cc(-c8cnc9cccc(Br)c9n8)cn7)CC6)c5)n4)cccc3C2=O)C(=O)N1] | 3-(4-((2-((3-((4-(4-(8-bromoquinoxalin- 2-yl)-1H-pyrazol-1-yl)piperidin-1- yl)methyl)phenyl)amino)pyrimidin-4- yl)amino)-1-oxoisoindolin-2- yl)piperidine-2,6-dione |
| GS-029 | 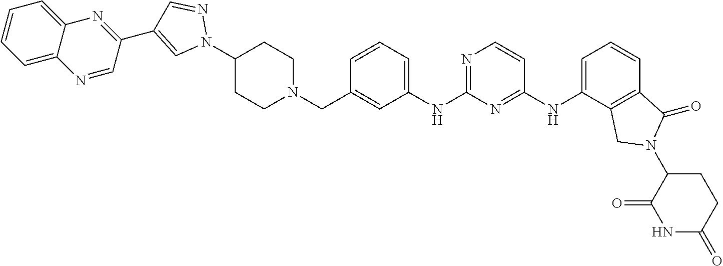 [SMILES: O=C1CCC(N2Cc3c(Nc4ccnc(Nc5cccc(CN6CCC(n7cc(-c8cnc9ccccc9n8)cn7)CC6)c5)n4)cccc3C2=O)C(=O)N1] | 3-(1-oxo-4-((2-((3-((4-(4-(quinoxalin-2- yl)-1H-pyrazol-1-yl)piperidin-1- yl)methyl)phenyl)amino)pyrimidin-4- yl)amino)isoindolin-2-yl)piperidine-2,6- dione |
| GS-030 | 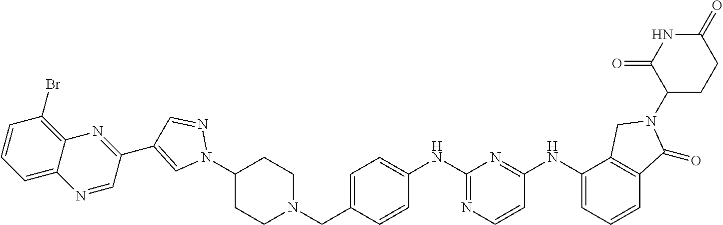 [SMILES: O=C1CCC(N2Cc3c(Nc4ccnc(Nc5ccc(CN6CCC(n7cc(-c8cnc9cccc(Br)c9n8)cn7)CC6)cc5)n4)cccc3C2=O)C(=O)N1] | 3-(4-((2-((4-((4-(4-(8-bromoquinoxalin- 2-yl)-1H-pyrazol-1-yl)piperidin-1- yl)methyl)phenyl)amino)pyrimidin-4- yl)amino)-1-oxoisoindolin-2- yl)piperidine-2,6-dione |
| GS-031 | 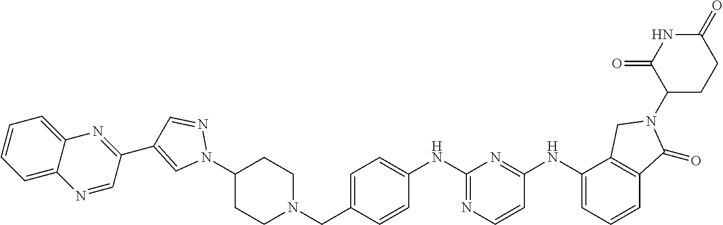 [SMILES: O=C1CCC(N2Cc3c(Nc4ccnc(Nc5ccc(CN6CCC(n7cc(-c8cnc9ccccc9n8)cn7)CC6)cc5)n4)cccc3C2=O)C(=O)N1] | 3-(1-oxo-4-((2-((4-((4-(4-(quinoxalin-2- yl)-1H-pyrazol-1-yl)piperidin-1- yl)methyl)phenyl)amino)pyrimidin-4- yl)amino)isoindolin-2-yl)piperidine-2,6- dione |
| GS-032 | 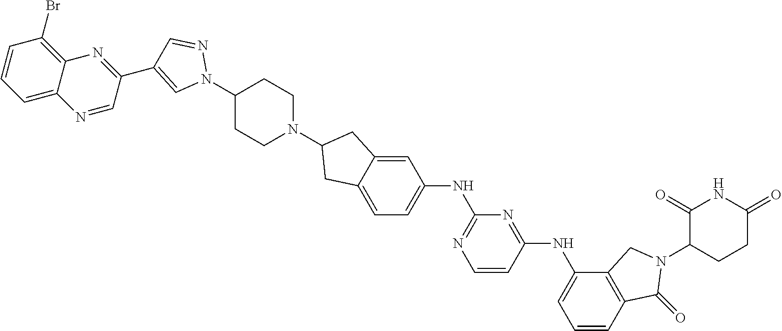 [SMILES: O=C1CCC(N2Cc3c(Nc4ccnc(Nc5ccc6c(c5)CC(N5CCC(n7cc(-c8cnc9cccc(Br)c9n8)cn7)CC5)C6)n4)cccc3C2=O)C(=O)N1] | 3-(4-((2-((2-(4-(4-(8-bromoquinoxalin-2- yl)-1H-pyrazol-1-yl)piperidin-1-yl)-2,3- dihydro-1H-inden-5-yl)amino)pyrimidin- 4-yl)amino)-1-oxoisoindolin-2- yl)piperidine-2,6-dione |
| GS-033 | 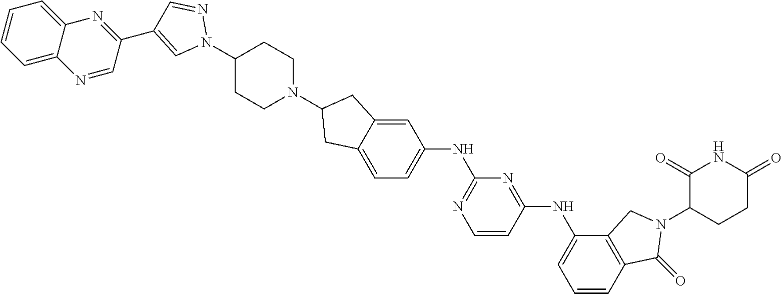 [SMILES: O=C1CCC(N2Cc3c(Nc4ccnc(Nc5ccc6c(c5)CC(N5CCC(n7cc(-c8cnc9ccccc9n8)cn7)CC5)C6)n4)cccc3C2=O)C(=O)N1] | 3-(1-oxo-4-((2-((2-(4-(4-(quinoxalin-2- yl)-1H-pyrazol-1-yl)piperidin-1-yl)-2,3- dihydro-1H-inden-5-yl)amino)pyrimidin- 4-yl)amino)isoindolin-2-yl)piperidine- 2,6-dione |
| GS-034 | 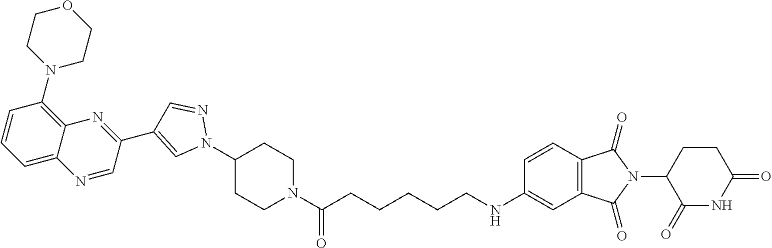 [SMILES: O=C1CCC(N2C(=O)c3ccc(NCCCCCC(=O)N4CCC(n5cc(-c6cnc7cccc(N8CCOCC8)c7n6)cn5)CC4)cc3C2=O)C(=O)N1] | 2-(2,6-dioxopiperidin-3-yl)-5-((6-(4-(4- (8-morpholinoquinoxalin-2-yl)-1H- pyrazol-1-yl)piperidin-1-yl)-6- oxohexyl)amino)isoindoline-1,3-dione |
| GS-035 | 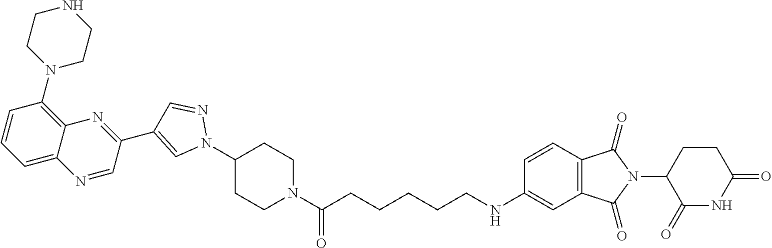 [SMILES: O=C1CCC(N2C(=O)c3ccc(NCCCCCC(=O)N4CCC(n5cc(-c6cnc7cccc(N8CCNCC8)c7n6)cn5)CC4)cc3C2=O)C(=O)N1] | 2-(2,6-dioxopiperidin-3-yl)-5-((6-ox0-6- (4-(4-(8-(piperazin-1-yl)quinoxalin-2-yl)- 1H-pyrazol-1-yl)piperidin-1- yl)hexyl)amino)isoindoline-1,3-dione |
| GS-036 | 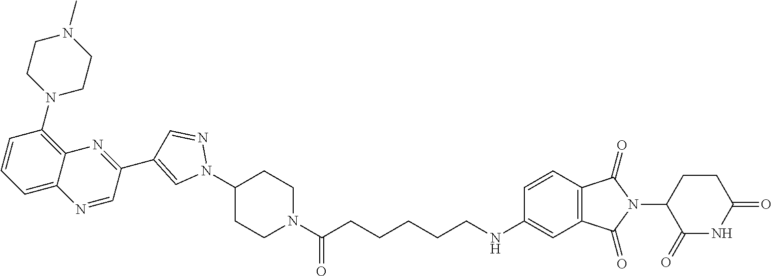 [SMILES: CN1CCN(c2cccc3ncc(-c4cnn(C5CCN(C(=O)CCCCCNc6ccc7c(c6)C(=O)N(C6CCC(=O)NC6=O)C7=O)CC5)c4)nc23)CC1] | 2-(2,6-dioxopiperidin-3-yl)-5-((6-(4-(4- (8-(4-methylpiperazin-1-yl)quinoxalin-2- yl)-1H-pyrazol-1-yl)piperidin-1-yl)-6- oxohexyl)amino)isoindoline-1,3-dione |
| GS-037 | 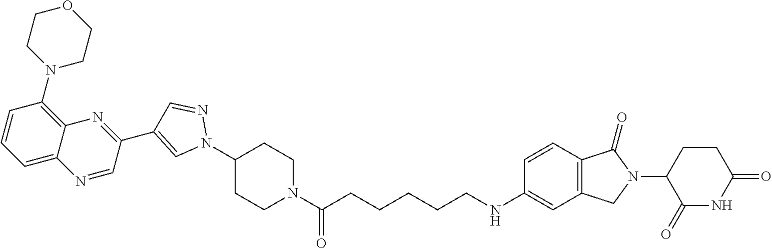 [SMILES: O=C1CCC(N2Cc3cc(NCCCCCC(=O)N4CCC(n5cc(-c6cnc7cccc(N8CCOCC8)c7n6)cn5)CC4)ccc3C2=O)C(=O)N1] | 3-(5-((6-(4-(4-(8-morpholinoquinoxalin- 2-yl)-1H-pyrazol-1-yl)piperidin-1-yl)-6- oxohexyl)amino)-1-oxoisoindolin-2- yl)piperidine-2,6-dione |
| GS-038 | 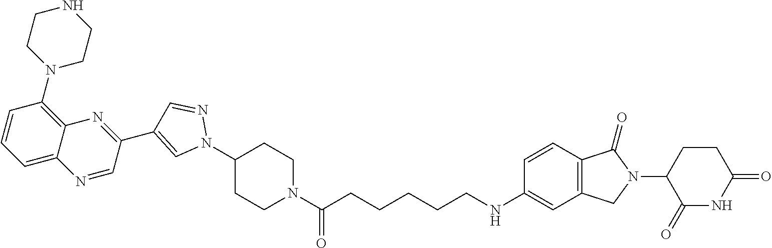 [SMILES: O=C1CCC(N2Cc3cc(NCCCCCC(=O)N4CCC(n5cc(-c6cnc7cccc(N8CCNCC8)c7n6)cn5)CC4)ccc3C2=O)C(=O)N1] | 3-(1-oxo-5-((6-oxo-6-(4-(4-(8-(piperazin- 1-yl)quinoxalin-2-yl)-1H-pyrazol-1- yl)piperidin-1-yl)hexyl)amino)isoindolin- 2-yl)piperidine-2,6-dione |
| GS-039 | 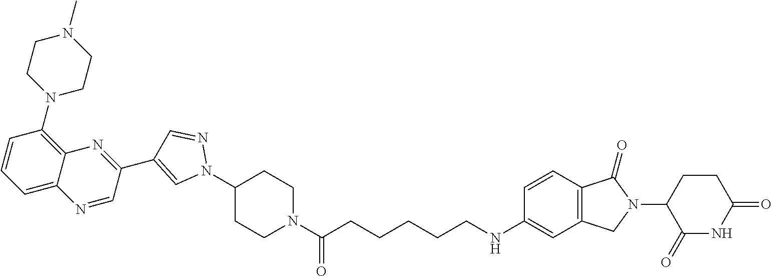 [SMILES: CN1CCN(c2cccc3ncc(-c4cnn(C5CCN(C(=O)CCCCCNc6ccc7c(c6)CN(C6CCC(=O)NC6=O)C7=O)CC5)c4)nc23)CC1] | 3-(5-((6-(4-(4-(8-(4-methylpiperazin-1- yl)quinoxalin-2-yl)-1H-pyrazol-1- yl)piperidin-1-yl)-6-oxohexyl)amino)-1- oxoisoindolin-2-yl)piperidine-2,6-dione |
| GS-040 | 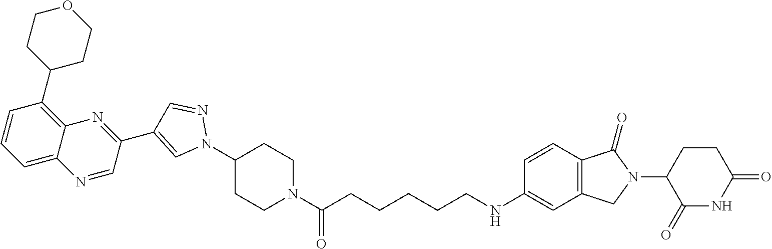 [SMILES: O=C1CCC(N2Cc3cc(NCCCCCC(=O)N4CCC(n5cc(-c6cnc7cccc(C8CCOCC8)c7n6)cn5)CC4)ccc3C2=O)C(=O)N1] | 3-(1-oxo-5-((6-oxo-6-(4-(4-(8- (tetrahydro-2H-pyran-4-yl)quinoxalin-2- yl)-1H-pyrazol-1-yl)piperidin-1- yl)hexyl)amino)isoindolin-2- yl)piperidine-2,6-dione |
| GS-041 | 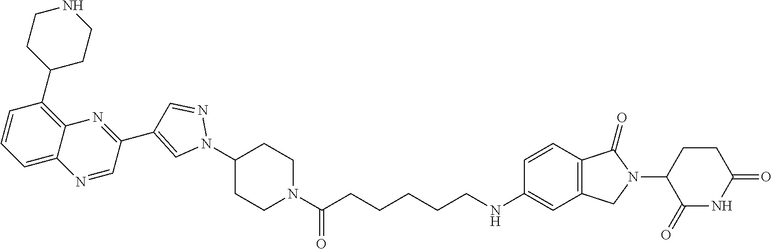 [SMILES: O=C1CCC(N2Cc3cc(NCCCCCC(=O)N4CCC(n5cc(-c6cnc7cccc(C8CCNCC8)c7n6)cn5)CC4)ccc3C2=O)C(=O)N1] | 3-(1-oxo-5-((6-oxo-6-(4-(4-(8-(piperidin- 4-yl)quinoxalin-2-yl)-1H-pyrazol-1- yl)piperidin-1-yl)hexyl)amino)isoindolin- 2-yl)piperidine-2,6-dione |
| GS-042 | 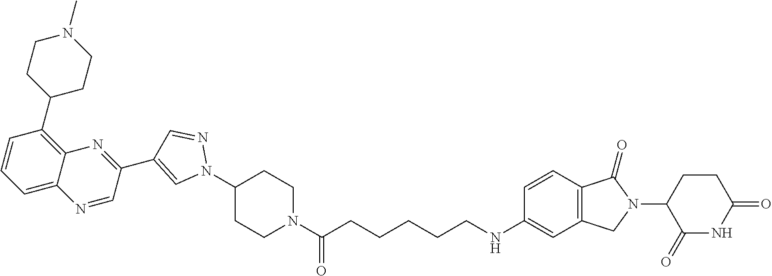 [SMILES: CN1CCC(c2cccc3ncc(-c4cnn(C5CCN(C(=O)CCCCCNc6ccc7c(c6)CN(C6CCC(=O)NC6=O)C7=O)CC5)c4)nc23)CC1] | 3-(5-((6-(4-(4-(8-(1-methylpiperidin-4- yl)quinoxalin-2-yl)-1H-pyrazol-1- yl)piperidin-1-yl)-6-oxohexyl)amino)-1- oxoisoindolin-2-yl)piperidine-2,6-dione |
| GS-043 | 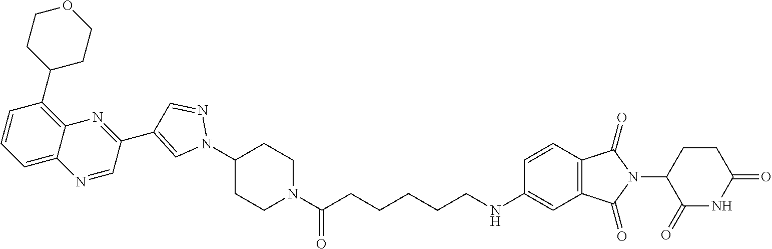 [SMILES: O=C1CCC(N2C(=O)c3ccc(NCCCCCC(=O)N4CCC(n5cc(-c6cnc7cccc(C8CCOCC8)c7n6)cn5)CC4)cc3C2=O)C(=O)N1] | 2-(2,6-dioxopiperidin-3-yl)-5-((6-oxo-6- (4-(4-(8-(tetrahydro-2H-pyran-4- yl)quinoxalin-2-yl)-1H-pyrazol-1- yl)piperidin-1- yl)hexyl)amino)isoindoline-1,3-dione |
| GS-044 | 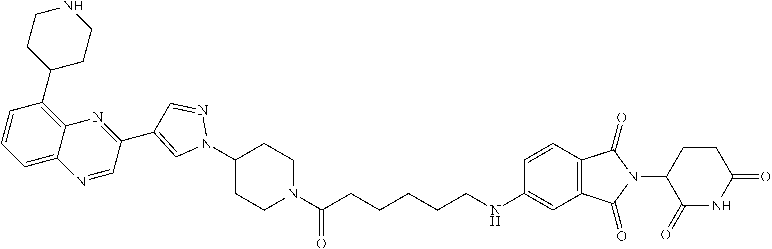 [SMILES: O=C1CCC(N2C(=O)c3ccc(NCCCCCC(=O)N4CCC(n5cc(-c6cnc7cccc(C8CCNCC8)c7n6)cn5)CC4)cc3C2=O)C(=O)N1] | 2-(2,6-dioxopiperidin-3-yl)-5-((6-oxo-6- (4-(4-(8-(piperidin-4-yl)quinoxalin-2-yl)- 1H-pyrazol-1-yl)piperidin-1- yl)hexyl)amino)isoindoline-1,3-dione |
| GS-045 | 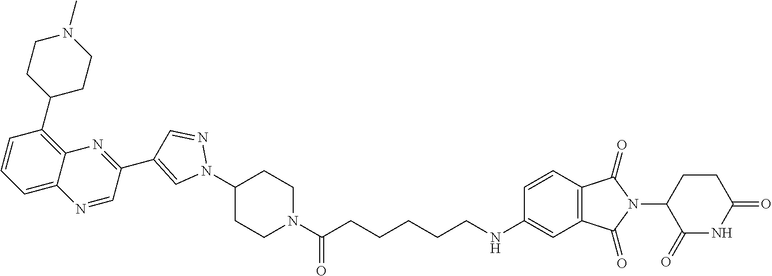 [SMILES: CN1CCC(c2cccc3ncc(-c4cnn(C5CCN(C(=O)CCCCCNc6ccc7c(c6)C(=O)N(C6CCC(=O)NC6=O)C7=O)CC5)c4)nc23)CC1] | 2-(2,6-dioxopiperidin-3-yl)-5-((6-(4-(4- (8-(1-methylpiperidin-4-yl)quinoxalin-2- yl)-1H-pyrazol-1-yl)piperidin-1-yl)-6- oxohexyl)amino)isoindoline-1,3-dione |
| GS-046 | 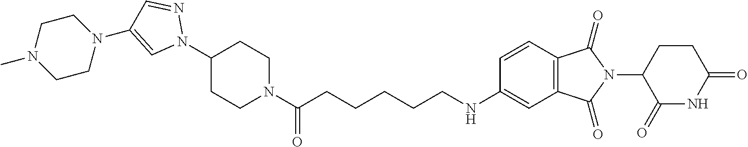 [SMILES: CN1CCN(c2cnn(C3CCN(C(=O)CCCCCNc4ccc5c(c4)C(=O)N(C4CCC(=O)NC4=O)C5=O)CC3)c2)CC1] | 2-(2,6-dioxopiperidin-3-yl)-5-((6-(4-(4- (4-methylpiperazin-1-yl)-1H-pyrazol-1- yl)piperidin-1-yl)-6- oxohexyl)amino)isoindoline-1,3-dione |
| GS-047 | 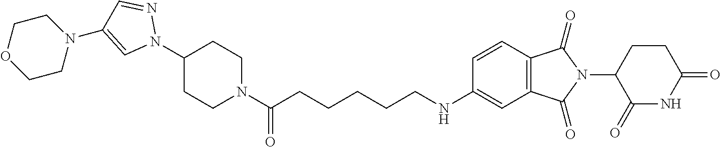 [SMILES: O=C1CCC(N2C(=O)c3ccc(NCCCCCC(=O)N4CCC(n5cc(N6CCOCC6)cn5)CC4)cc3C2=O)C(=O)N1] | 2-(2,6-dioxopiperidin-3-yl)-5-((6-(4-(4- morpholino-1H-pyrazol-1-yl)piperidin-1- yl)-6-oxohexyl)amino)isoindoline-1,3- dione |
| GS-048 | 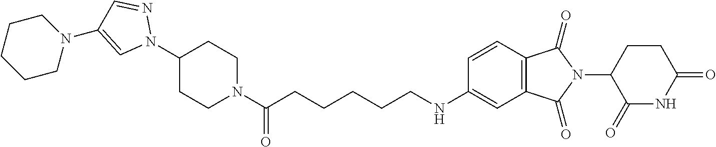 [SMILES: O=C1CCC(N2C(=O)c3ccc(NCCCCCC(=O)N4CCC(n5cc(N6CCCCC6)cn5)CC4)cc3C2=O)C(=O)N1] | 2-(2,6-dioxopiperidin-3-yl)-5-((6-oxo-6- (4-(4-(piperidin-1-yl)-1H-pyrazol-1- yl)piperidin-1- yl)hexyl)amino)isoindoline-1,3-dione |
| GS-049 | 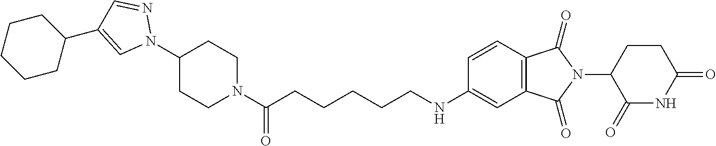 [SMILES: O=C1CCC(N2C(=O)c3ccc(NCCCCCC(=O)N4CCC(n5cc(C6CCCCC6)cn5)CC4)cc3C2=O)C(=O)N1] | 5-((6-(4-(4-cyclohexyl-1H-pyrazol-1- yl)piperidin-1-yl)-6-oxohexyl)amino)-2- (2,6-dioxopiperidin-3-yl)isoindoline-1,3- dione |
| GS-050 | 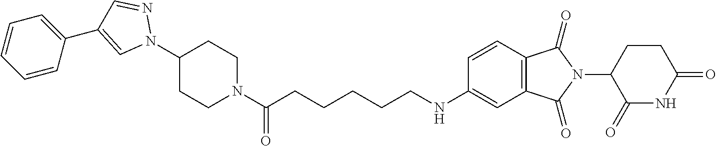 [SMILES: O=C1CCC(N2C(=O)c3ccc(NCCCCCC(=O)N4CCC(n5cc(-c6ccccc6)cn5)CC4)cc3C2=O)C(=O)N1] | 2-(2,6-dioxopiperidin-3-yl)-5-((6-oxo-6- (4-(4-phenyl-1H-pyrazol-1-yl)piperidin- 1-yl)hexyl)amino)isoindoline-1,3-dione |
| GS-051 | 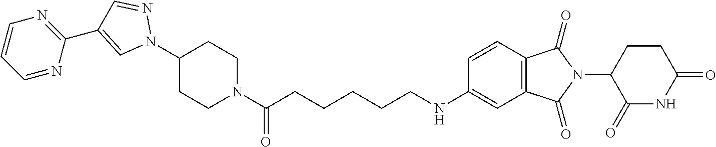 [SMILES: O=C1CCC(N2C(=O)c3ccc(NCCCCCC(=O)N4CCC(n5cc(-c6ncccn6)cn5)CC4)cc3C2=O)C(=O)N1] | 2-(2,6-dioxopiperidin-3-yl)-5-((6-oxo-6- (4-(4-(pyrimidin-2-yl)-1H-pyrazol-1- yl)piperidin-1- yl)hexyl)amino)isoindoline-1,3-dione |
| GS-052 | 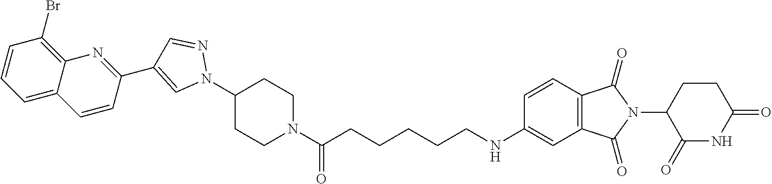 [SMILES: O=C1CCC(N2C(=O)c3ccc(NCCCCCC(=O)N4CCC(n5cc(-c6ccc7cccc(Br)c7n6)cn5)CC4)cc3C2=O)C(=O)N1] | 5-((6-(4-(4-(8-bromoquinolin-2-yl)-1H- pyrazol-1-yl)piperidin-1-yl)-6- oxohexyl)amino)-2-(2,6-dioxopiperidin- 3-yl)isoindoline-1,3-dione |
| GS-053 | 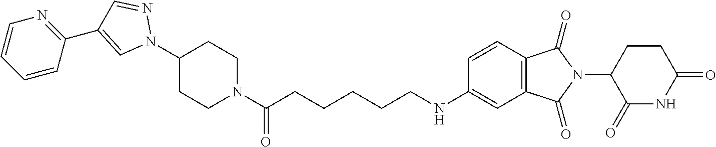 [SMILES: O=C1CCC(N2C(=O)c3ccc(NCCCCCC(=O)N4CCC(n5cc(-c6ccccn6)cn5)CC4)cc3C2=O)C(=O)N1] | 2-(2,6-dioxopiperidin-3-yl)-5-((6-oxo-6- (4-(4-(pyridin-2-yl)-1H-pyrazol-1- yl)piperidin-1- yl)hexyl)amino)isoindoline-1,3-dione |
| GS-054 | 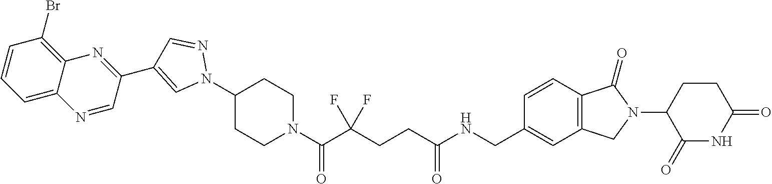 [SMILES: O=C(CCC(F)(F)C(=O)N1CCC(n2cc(-c3cnc4cccc(Br)c4n3)cn2)CC1)NCc1ccc2c(c1)CN(C1CCC(=O)NC1=O)C2=O] | 5-(4-(4-(8-bromoquinoxalin-2-yl)-1H- pyrazol-1-yl)piperidin-1-yl)-N-((2-(2,6- dioxopiperidin-3-yl)-1-oxoisoindolin-5- yl)methyl)-4,4-difluoro-5- oxopentanamide |
| GS-055 | 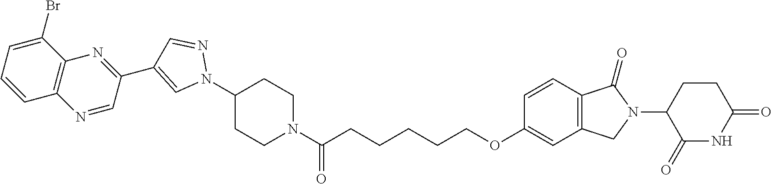 [SMILES: O=C1CCC(N2Cc3cc(OCCCCCC(=O)N4CCC(n5cc(-c6cnc7cccc(Br)c7n6)cn5)CC4)ccc3C2=O)C(=O)N1] | 3-(5-((6-(4-(4-(8-bromoquinoxalin-2-yl)- 1H-pyrazol-1-yl)piperidin-1-yl)-6- oxohexyl)oxy)-1-oxoisoindolin-2- yl)piperidine-2,6-dione |
| GS-056 | 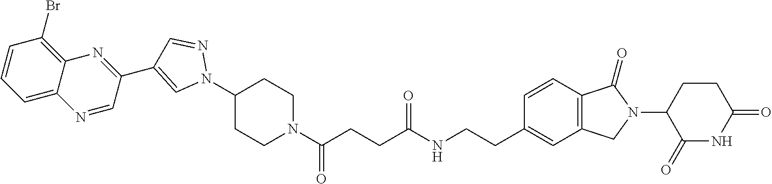 [SMILES: O=C(CCC(=O)N1CCC(n2cc(-c3cnc4cccc(Br)c4n3)cn2)CC1)NCCc1ccc2c(c1)CN(C1CCC(=O)NC1=O)C2=O] | 4-(4-(4-(8-bromoquinoxalin-2-yl)-1H- pyrazol-1-yl)piperidin-1-yl)-N-(2-(2-(2,6- dioxopiperidin-3-yl)-1-oxoisoindolin-5- yl)ethyl)-4-oxobutanamide |
| GS-057 | 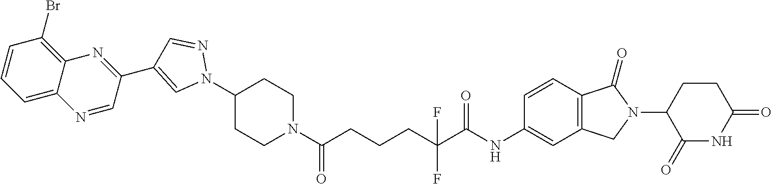 [SMILES: O=C1CCC(N2Cc3cc(NC(=O)C(F)(F)CCCC(=O)N4CCC(n5cc(-c6cnc7cccc(Br)c7n6)cn5)CC4)ccc3C2=O)C(=O)N1] | 6-(4-(4-(8-bromoquinoxalin-2-yl)-1H- pyrazol-1-yl)piperidin-1-yl)-N-(2-(2,6- dioxopiperidin-3-yl)-1-oxoisoindolin-5- yl)-2,2-difluoro-6-oxohexanamide |
| GS-058 | 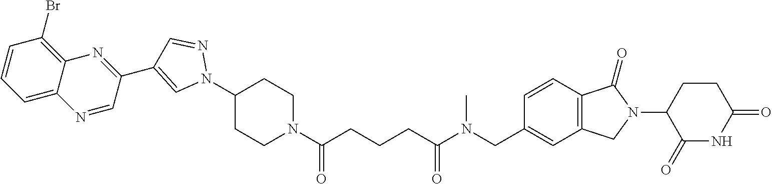 [SMILES: CN(Cc1ccc2c(c1)CN(C1CCC(=O)NC1=O)C2=O)C(=O)CCCC(=O)N1CCC(n2cc(-c3cnc4cccc(Br)c4n3)cn2)CC1] | 5-(4-(4-(8-bromoquinoxalin-2-yl)-1H- pyrazol-1-yl)piperidin-1-yl)-N-((2-(2,6- dioxopiperidin-3-yl)-1-oxoisoindolin-5- yl)methyl)-N-methyl-5-oxopentanamide |
| GS-059 | 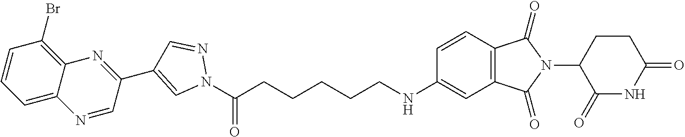 [SMILES: O=C1CCC(N2C(=O)c3ccc(NCCCCCC(=O)n4cc(-c5cnc6cccc(Br)c6n5)cn4)cc3C2=O)C(=O)N1] | 5-((6-(4-(8-bromoquinoxalin-2-yl)-1H- pyrazol-1-yl)-6-oxohexyl)amino)-2-(2,6- dioxopiperidin-3-yl)isoindoline-1,3-dione |
| GS-060 | 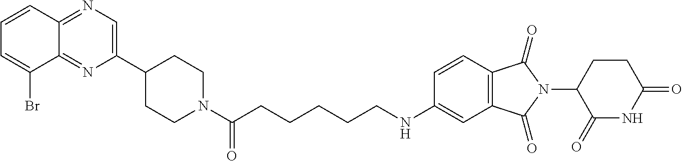 [SMILES: O=C1CCC(N2C(=O)c3ccc(NCCCCCC(=O)N4CCC(c5cnc6cccc(Br)c6n5)CC4)cc3C2=O)C(=O)N1] | 5-((6-(4-(8-bromoquinoxalin-2- yl)piperidin-1-yl)-6-oxohexyl)amino)-2- (2,6-dioxopiperidin-3-yl)isoindoline-1,3- dione |
| GS-061 | 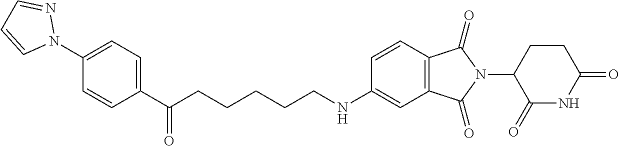 [SMILES: O=C1CCC(N2C(=O)c3ccc(NCCCCCC(=O)c4ccc(-n5cccn5)cc4)cc3C2=O)C(=O)N1] | 5-((6-(4-(1H-pyrazol-1-yl)phenyl)-6- oxohexyl)amino)-2-(2,6-dioxopiperidin- 3-yl)isoindoline-1,3-dione |
| GS-062 | 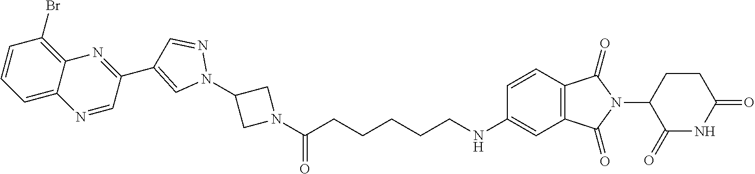 [SMILES: O=C1CCC(N2C(=O)c3ccc(NCCCCCC(=O)N4CC(n5cc(-c6cnc7cccc(Br)c7n6)cn5)C4)cc3C2=O)C(=O)N1] | 5-((6-(3-(4-(8-bromoquinoxalin-2-yl)- 1H-pyrazol-1-yl)azetidin-1-yl)-6- oxohexyl)amino)-2-(2,6-dioxopiperidin- 3-yl)isoindoline-1,3-dione |
| GS-063 | 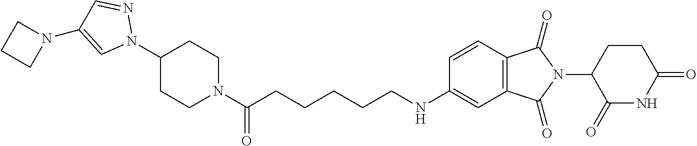 [SMILES: O=C1CCC(N2C(=O)c3ccc(NCCCCCC(=O)N4CCC(n5cc(N6CCC6)cn5)CC4)cc3C2=O)C(=O)N1] | 5-((6-(4-(4-(azetidin-1-yl)-1H-pyrazol-1- yl)piperidin-1-yl)-6-oxohexyl)amino)-2- (2,6-dioxopiperidin-3-yl)isoindoline-1,3- dione |
| GS-064 | 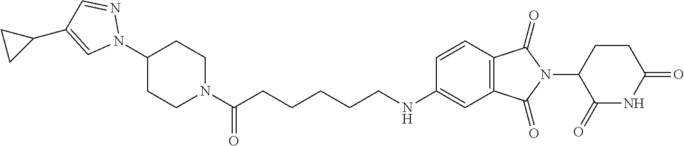 [SMILES: O=C1CCC(N2C(=O)c3ccc(NCCCCCC(=O)N4CCC(n5cc(C6CC6)cn5)CC4)cc3C2=O)C(=O)N1] | 5-((6-(4-(4-cyclopropyl-1H-pyrazol-1- yl)piperidin-1-yl)-6-oxohexyl)amino)-2- (2,6-dioxopiperidin-3-yl)isoindoline-1,3- dione |
| GS-065 | 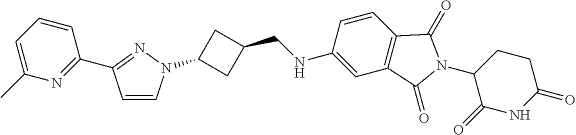 [SMILES: Cc1cccc(-c2ccn([C@H]3C[C@H](CNc4ccc5c(c4)C(=O)N(C4CCC(=O)NC4=O)C5=O)C3)n2)n1] | 2-(2,6-dioxopiperidin-3-yl)-5-(((trans-3- (3-(6-methylpyridin-2-yl)-1H-pyrazol-1- yl)cyclobutyl)methyl)amino)isoindoline- 1,3-dione |
| GS-066 | 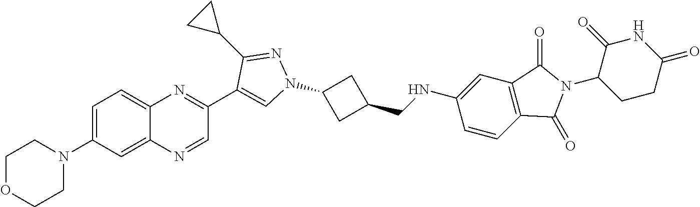 [SMILES: O=C1CCC(N2C(=O)c3ccc(NC[C@H]4C[C@H](n5cc(-c6cnc7cc(N8CCOCC8)ccc7n6)c(C6CC6)n5)C4)cc3C2=O)C(=O)N1] | 5-(((trans-3-(3-cyclopropyl-4-(6- morpholinoquinoxalin-2-yl)-1H-pyrazol- 1-yl)cyclobutyl)methyl)amino)-2-(2,6- dioxopiperidin-3-yl)isoindoline-1,3-dione |
| GS-067 | 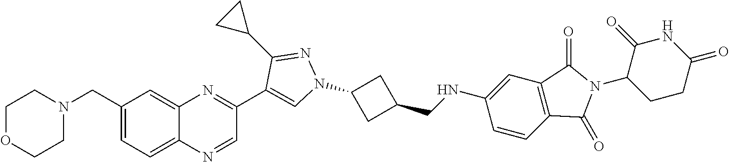 [SMILES: O=C1CCC(N2C(=O)c3ccc(NC[C@H]4C[C@H](n5cc(-c6cnc7ccc(CN8CCOCC8)cc7n6)c(C6CC6)n5)C4)cc3C2=O)C(=O)N1] | 5-(((trans-3-(3-cyclopropyl-4-(7- (morpholinomethyl)quinoxalin-2-yl)-1H- pyrazol-1-yl)cyclobutyl)methyl)amino)- 2-(2,6-dioxopiperidin-3-yl)isoindoline- 1,3-dione |
| GS-068 | 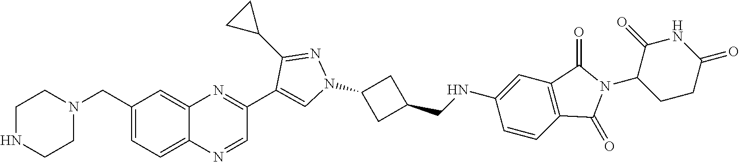 [SMILES: O=C1CCC(N2C(=O)c3ccc(NC[C@H]4C[C@H](n5cc(-c6cnc7ccc(CN8CCNCC8)cc7n6)c(C6CC6)n5)C4)cc3C2=O)C(=O)N1] | 5-(((trans-3-(3-cyclopropyl-4-(7- (piperazin-1-ylmethyl)quinoxalin-2-yl)- 1H-pyrazol-1- yl)cyclobutyl)methyl)amino)-2-(2,6- dioxopiperidin-3-yl)isoindoline-1,3-dione |
| GS-069 | 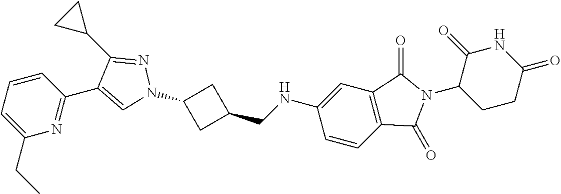 [SMILES: CCc1cccc(-c2cn([C@H]3C[C@H](CNc4ccc5c(c4)C(=O)N(C4CCC(=O)NC4=O)C5=O)C3)nc2C2CC2)n1] | 5-(((trans-3-(3-cyclopropyl-4-(6- ethylpyridin-2-yl)-1H-pyrazol-1- yl)cyclobutyl)methyl)amino)-2-(2,6- dioxopiperidin-3-yl)isoindoline-1,3-dione |
| GS-070 | 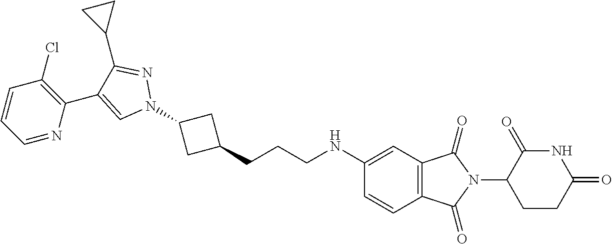 [SMILES: O=C1CCC(N2C(=O)c3ccc(NCCC[C@H]4C[C@H](n5cc(-c6ncccc6Cl)c(C6CC6)n5)C4)cc3C2=O)C(=O)N1] | 5-((3-(trans-3-(4-(3-chloropyridin-2-yl)- 3-cyclopropyl-1H-pyrazol-1- yl)cyclobutyl)propyl)amino)-2-(2,6- dioxopiperidin-3-yl)isoindoline-1,3-dione |
| GS-071 | 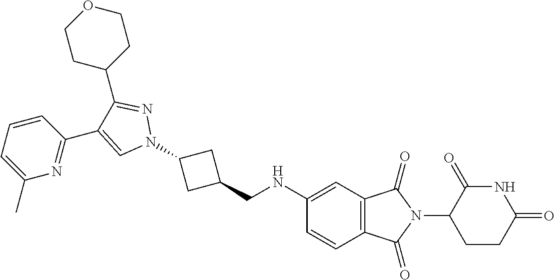 [SMILES: Cc1cccc(-c2cn([C@H]3C[C@H](CNc4ccc5c(c4)C(=O)N(C4CCC(=O)NC4=O)C5=O)C3)nc2C2CCOCC2)n1] | 2-(2,6-dioxopiperidin-3-yl)-5-(((trans-3- (4-(6-methylpyridin-2-yl)-3-(tetrahydro- 2H-pyran-4-yl)-1H-pyrazol-1- yl)cyclobutyl)methyl)amino)isoindoline- 1,3-dione |
| GS-072 | 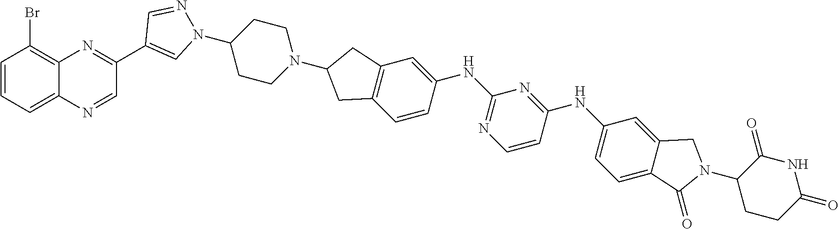 [SMILES: O=C1CCC(N2Cc3cc(Nc4ccnc(Nc5ccc6c(c5)CC(N5CCC(n7cc(-c8cnc9cccc(Br)c9n8)cn7)CC5)C6)n4)ccc3C2=O)C(=O)N1] | 3-(5-((2-((2-(4-(4-(8-bromoquinoxalin-2- yl)-1H-pyrazol-1-yl)piperidin-1-yl)-2,3- dihydro-1H-inden-5-yl)amino)pyrimidin- 4-yl)amino)-1-oxoisoindolin-2- yl)piperidine-2,6-dione |
| GS-073 | 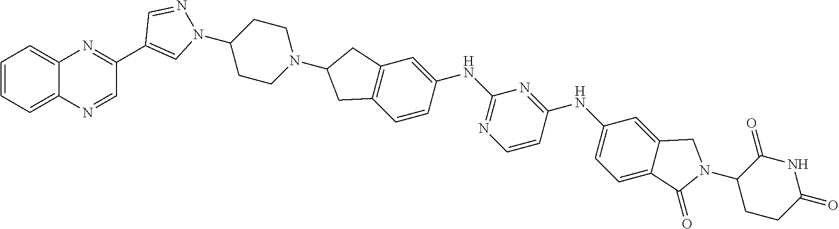 [SMILES: O=C1CCC(N2Cc3cc(Nc4ccnc(Nc5ccc6c(c5)CC(N5CCC(n7cc(-c8cnc9ccccc9n8)cn7)CC5)C6)n4)ccc3C2=O)C(=O)N1] | 3-(1-oxo-5-((2-((2-(4-(4-(quinoxalin-2- yl)-1H-pyrazol-1-yl)piperidin-1-yl)-2,3- dihydro-1H-inden-5-yl)amino)pyrimidin- 4-yl)amino)isoindolin-2-yl)piperidine- 2,6-dione |
| GS-074 | 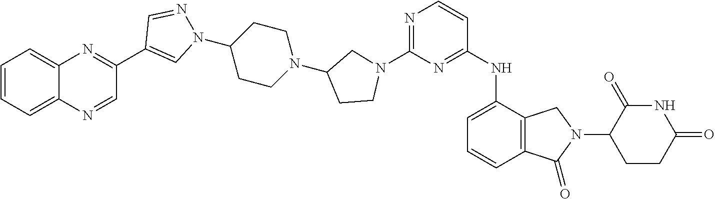 [SMILES: O=C1CCC(N2Cc3c(Nc4ccnc(N5CCC(N6CCC(n7cc(-c8cnc9ccccc9n8)cn7)CC6)C5)n4)cccc3C2=O)C(=O)N1] | 3-(1-oxo-4-((2-(3-(4-(4-(quinoxalin-2- yl)-1H-pyrazol-1-yl)piperidin-1- yl)pyrrolidin-1-yl)pyrimidin-4- yl)amino)isoindolin-2-yl)piperidine-2,6- dione |
| GS-075 | 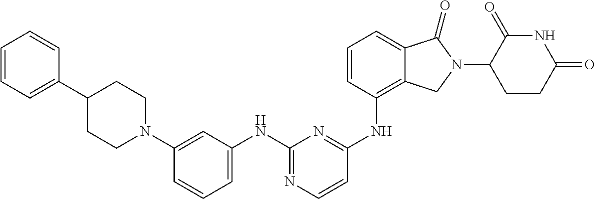 [SMILES: O=C1CCC(N2Cc3c(Nc4ccnc(Nc5cccc(N6CCC(c7ccccc7)CC6)c5)n4)cccc3C2=O)C(=O)N1] | 3-(1-oxo-4-((2-((3-(4-phenylpiperidin-1- yl)phenyl)amino)pyrimidin-4- yl)amino)isoindolin-2-yl)piperidine-2,6- dione |
| GS-076 | 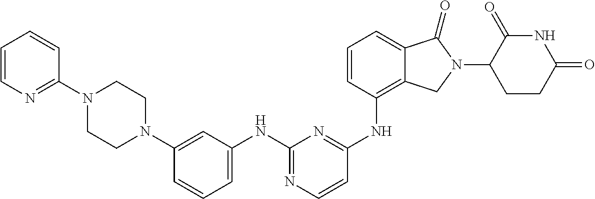 [SMILES: O=C1CCC(N2Cc3c(Nc4ccnc(Nc5cccc(N6CCN(c7ccccn7)CC6)c5)n4)cccc3C2=O)C(=O)N1] | 3-(1-oxo-4-((2-((3-(4-(pyridin-2- yl)piperazin-1- yl)phenyl)amino)pyrimidin-4- yl)amino)isoindolin-2-yl)piperidine-2,6- dione |
| GS-077 | 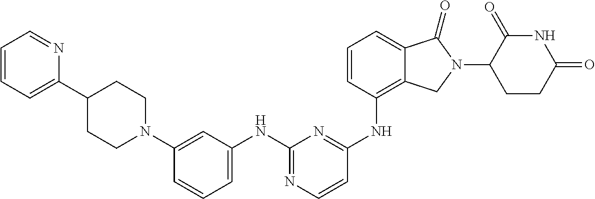 [SMILES: O=C1CCC(N2Cc3c(Nc4ccnc(Nc5cccc(N6CCC(c7ccccn7)CC6)c5)n4)cccc3C2=O)C(=O)N1] | 3-(1-oxo-4-((2-((3-(4-(pyridin-2- yl)piperidin-1- yl)phenyl)amino)pyrimidin-4- yl)amino)isoindolin-2-yl)piperidine-2,6- dione |
| GS-078 | 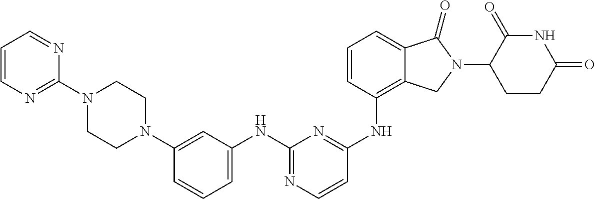 [SMILES: O=C1CCC(N2Cc3c(Nc4ccnc(Nc5cccc(N6CCN(c7ncccn7)CC6)c5)n4)cccc3C2=O)C(=O)N1] | 3-(1-oxo-4-((2-((3-(4-(pyrimidin-2- yl)piperazin-1- yl)phenyl)amino)pyrimidin-4- yl)amino)isoindolin-2-yl)piperidine-2,6- dione |
| GS-079 | 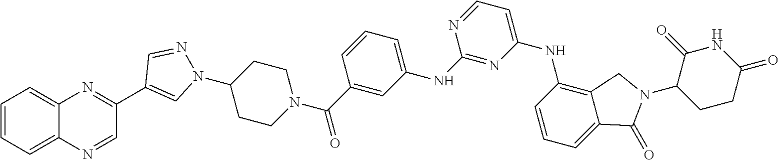 [SMILES: O=C1CCC(N2Cc3c(Nc4ccnc(Nc5cccc(C(=O)N6CCC(n7cc(-c8cnc9ccccc9n8)cn7)CC6)c5)n4)cccc3C2=O)C(=O)N1] | 3-(1-oxo-4-((2-((3-(4-(4-(quinoxalin-2- yl)-1H-pyrazol-1-yl)piperidine-1- carbonyl)phenyl)amino)pyrimidin-4- yl)amino)isoindolin-2-yl)piperidine-2,6- dione |
| GS-080 | 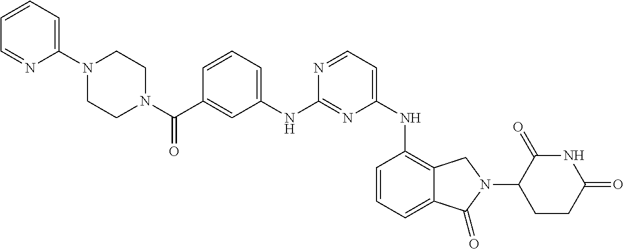 [SMILES: O=C1CCC(N2Cc3c(Nc4ccnc(Nc5cccc(C(=O)N6CCN(c7ccccn7)CC6)c5)n4)cccc3C2=O)C(=O)N1] | 3-(1-oxo-4-((2-((3-(4-(pyridin-2- yl)piperazine-1- carbonyl)phenyl)amino)pyrimidin-4- yl)amino)isoindolin-2-yl)piperidine-2,6- dione |
| GS-081 | 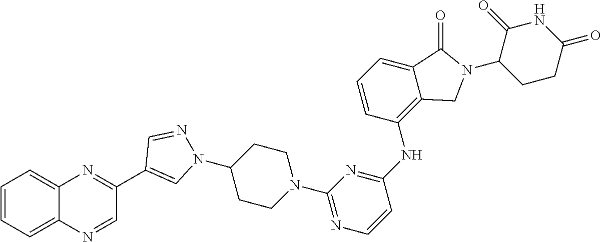 [SMILES: O=C1CCC(N2Cc3c(Nc4ccnc(N5CCC(n6cc(-c7cnc8ccccc8n7)cn6)CC5)n4)cccc3C2=O)C(=O)N1] | 3-(1-oxo-4-((2-(4-(4-(quinoxalin-2-yl)- 1H-pyrazol-1-yl)piperidin-1- yl)pyrimidin-4-yl)amino)isoindolin-2- yl)piperidine-2,6-dione |
| GS-082 | 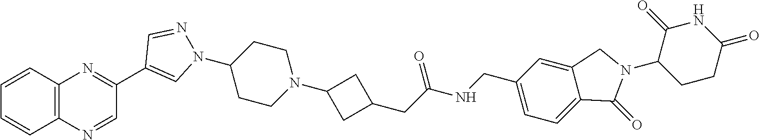 [SMILES: O=C(CC1CC(N2CCC(n3cc(-c4cnc5ccccc5n4)cn3)CC2)C1)NCc1ccc2c(c1)CN(C1CCC(=O)NC1=O)C2=O] | N-((2-(2,6-dioxopiperidin-3-yl)-1- oxoisoindolin-5-yl)methyl)-2-(3-(4-(4- (quinoxalin-2-yl)-1H-pyrazol-1- yl)piperidin-1-yl)cyclobutyl)acetamide |
| GS-083 | 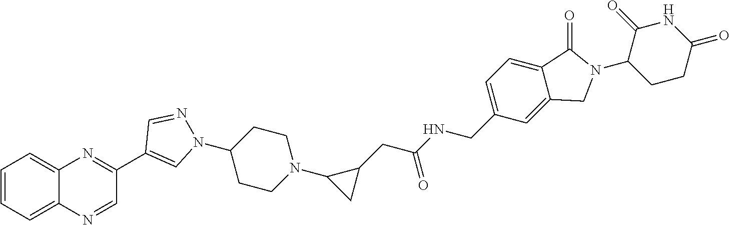 [SMILES: O=C(CC1CC1N1CCC(n2cc(-c3cnc4ccccc4n3)cn2)CC1)NCc1ccc2c(c1)CN(C1CCC(=O)NC1=O)C2=O] | N-((2-(2,6-dioxopiperidin-3-yl)-1- oxoisoindolin-5-yl)methyl)-2-(2-(4-(4- (quinoxalin-2-yl)-1H-pyrazol-1- yl)piperidin-1-yl)cyclopropyl)acetamide |
| GS-084 | 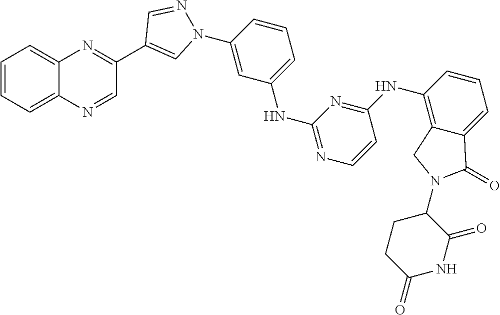 [SMILES: O=C1CCC(N2Cc3c(Nc4ccnc(Nc5cccc(-n6cc(-c7cnc8ccccc8n7)cn6)c5)n4)cccc3C2=O)C(=O)N1] | 3-(1-oxo-4-((2-((3-(4-(quinoxalin-2-yl)- 1H-pyrazol-1- yl)phenyl)amino)pyrimidin-4- yl)amino)isoindolin-2-yl)piperidine-2,6- dione |
| GS-085 | 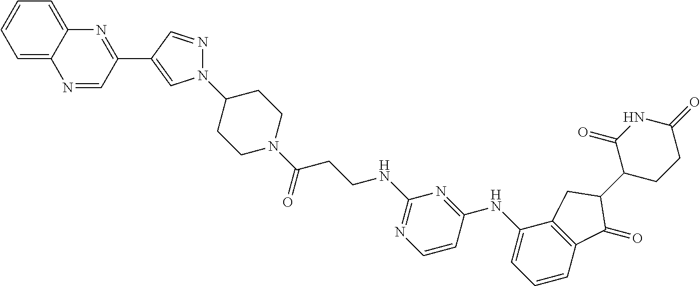 [SMILES: O=C1CCC(C2Cc3c(Nc4ccnc(NCCC(=O)N5CCC(n6cc(-c7cnc8ccccc8n7)cn6)CC5)n4)cccc3C2=O)C(=O)N1] | 3-(1-oxo-4-((2-((3-oxo-3-(4-(4- (quinoxalin-2-yl)-1H-pyrazol-1- yl)piperidin-1- yl)propyl)amino)pyrimidin-4- yl)amino)isoindolin-2-yl)piperidine-2,6- dione |
| GS-086 | 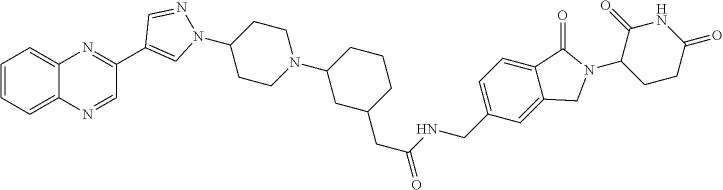 [SMILES: O=C(CC1CCCC(N2CCC(n3cc(-c4cnc5ccccc5n4)cn3)CC2)C1)NCc1ccc2c(c1)CN(C1CCC(=O)NC1=O)C2=O] | N-((2-(2,6-dioxopiperidin-3-yl)-1- oxoisoindolin-5-yl)methyl)-2-(3-(4-(4- (quinoxalin-2-yl)-1H-pyrazol-1- yl)piperidin-1-yl)cyclohexyl)acetamide |
| GS-087 | 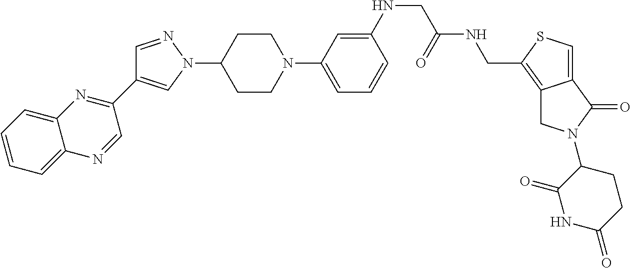 [SMILES: O=C(CNc1cccc(N2CCC(n3cc(-c4cnc5ccccc5n4)cn3)CC2)c1)NCc1scc2c1CN(C1CCC(=O)NC1=O)C2=O] | N-((5-(2,6-dioxopiperidin-3-yl)-4-oxo- 5,6-dihydro-4H-thieno[3,4-c]pyrrol-1- yl)methyl)-2-((3-(4-(4-(quinoxalin-2-yl)- 1H-pyrazol-1-yl)piperidin-1- yl)phenyl)amino)acetamide |
| GS-088 | 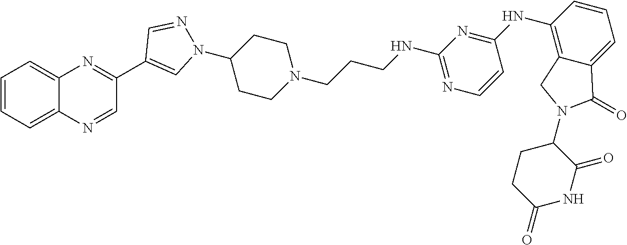 [SMILES: O=C1CCC(N2Cc3c(Nc4ccnc(NCCCN5CCC(n6cc(-c7cnc8ccccc8n7)cn6)CC5)n4)cccc3C2=O)C(=O)N1] | 3-(1-oxo-4-((2-((3-(4-(4-(quinoxalin-2- yl)-1H-pyrazol-1-yl)piperidin-1- yl)propyl)amino)pyrimidin-4- yl)amino)isoindolin-2-yl)piperidine-2,6- dione |
| GS-089 | 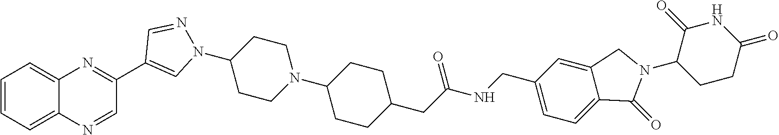 [SMILES: O=C(CC1CCC(N2CCC(n3cc(-c4cnc5ccccc5n4)cn3)CC2)CC1)NCc1ccc2c(c1)CN(C1CCC(=O)NC1=O)C2=O] | N-((2-(2,6-dioxopiperidin-3-yl)-1- oxoisoindolin-5-yl)methyl)-2-(4-(4-(4- (quinoxalin-2-yl)-1H-pyrazol-1- yl)piperidin-1-yl)cyclohexyl)acetamide |
| GS-090 | 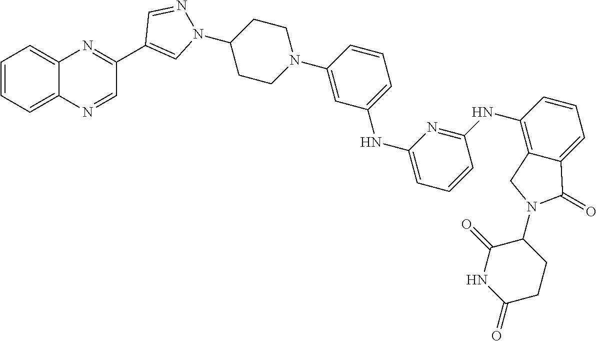 [SMILES: O=C1CCC(N2Cc3c(Nc4cccc(Nc5cccc(N6CCC(n7cc(-c8cnc9ccccc9n8)cn7)CC6)c5)n4)cccc3C2=O)C(=O)N1] | 3-(1-oxo-4-((6-((3-(4-(4-(quinoxalin-2- yl)-1H-pyrazol-1-yl)piperidin-1- yl)phenyl)amino)pyridin-2- yl)amino)isoindolin-2-yl)piperidine-2,6- dione |
| GS-091 | 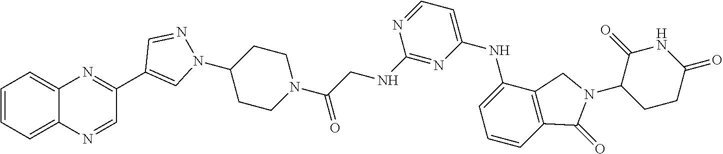 [SMILES: O=C1CCC(N2Cc3c(Nc4ccnc(NCC(=O)N5CCC(n6cc(-c7cnc8ccccc8n7)cn6)CC5)n4)cccc3C2=O)C(=O)N1] | 3-(1-oxo-4-((2-((2-oxo-2-(4-(4- (quinoxalin-2-yl)-1H-pyrazol-1- yl)piperidin-1-yl)ethyl)amino)pyrimidin- 4-yl)amino)isoindolin-2-yl)piperidine- 2,6-dione |
| GS-092 | 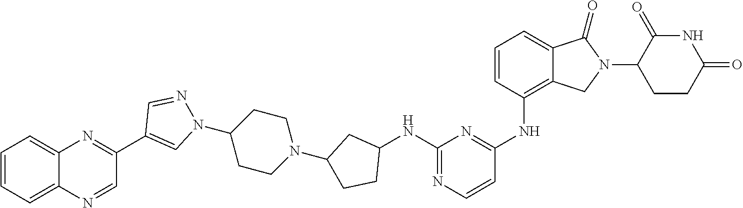 [SMILES: O=C1CCC(N2Cc3c(Nc4ccnc(NC5CCC(N6CCC(n7cc(-c8cnc9ccccc9n8)cn7)CC6)C5)n4)cccc3C2=O)C(=O)N1] | 3-(1-oxo-4-((2-((3-(4-(4-(quinoxalin-2- yl)-1H-pyrazol-1-yl)piperidin-1- yl)cyclopentyl)amino)pyrimidin-4- yl)amino)isoindolin-2-yl)piperidine-2,6- dione |
| GS-093 | 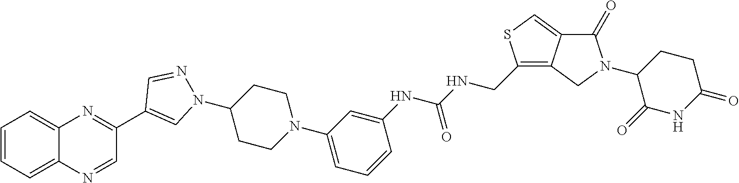 [SMILES: O=C1CCC(N2Cc3c(csc3CNC(=O)Nc3cccc(N4CCC(n5cc(-c6cnc7ccccc7n6)cn5)CC4)c3)C2=O)C(=O)N1] | 1-((5-(2,6-dioxopiperidin-3-yl)-4-oxo- 5,6-dihydro-4H-thieno[3,4-c]pyrrol-1- yl)methyl)-3-(3-(4-(4-(quinoxalin-2-yl)- 1H-pyrazol-1-yl)piperidin-1- yl)phenyl)urea |
| GS-094 | 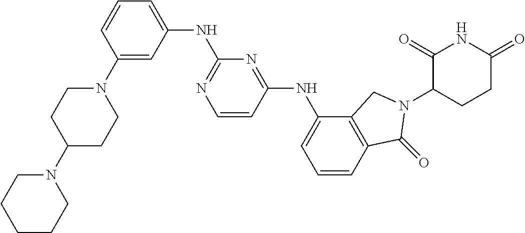 [SMILES: O=C1CCC(N2Cc3c(Nc4ccnc(Nc5cccc(N6CCC(N7CCCCC7)CC6)c5)n4)cccc3C2=O)C(=O)N1] | 3-(4-((2-((3-([1,4′-bipiperidin]-1′- yl)phenyl)amino)pyrimidin-4-yl)amino)- 1-oxoisoindolin-2-yl)piperidine-2,6-dione |
| GS-095 | 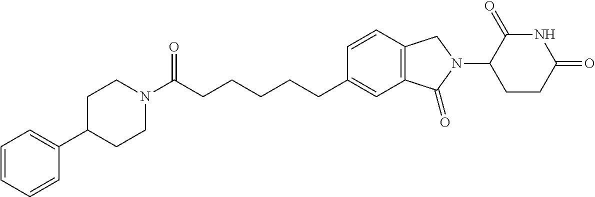 [SMILES: O=C1CCC(N2Cc3ccc(CCCCCC(=O)N4CCC(c5ccccc5)CC4)cc3C2=O)C(=O)N1] | 3-(1-oxo-6-(6-oxo-6-(4-phenylpiperidin- 1-yl)hexyl)isoindolin-2-yl)piperidine-2,6- dione |
| GS-096 | 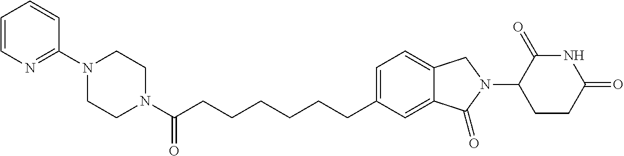 [SMILES: O=C1CCC(N2Cc3ccc(CCCCCCC(=O)N4CCN(c5ccccn5)CC4)cc3C2=O)C(=O)N1] | 3-(1-oxo-6-(7-oxo-7-(4-(pyridin-2- yl)piperazin-1-yl)heptyl)isoindolin-2- yl)piperidine-2,6-dione |
| GS-097 | 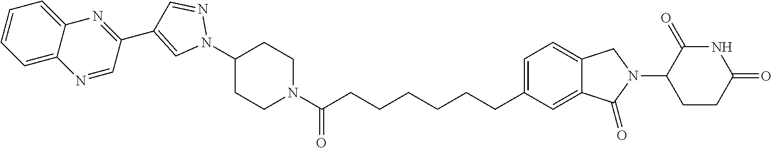 [SMILES: O=C1CCC(N2Cc3ccc(CCCCCCC(=O)N4CCC(n5cc(-c6cnc7ccccc7n6)cn5)CC4)cc3C2=O)C(=O)N1] | 3-(1-oxo-6-(7-oxo-7-(4-(4-(quinoxalin-2- yl)-1H-pyrazol-1-yl)piperidin-1- yl)heptyl)isoindolin-2-yl)piperidine-2,6- dione |
| GS-098 | 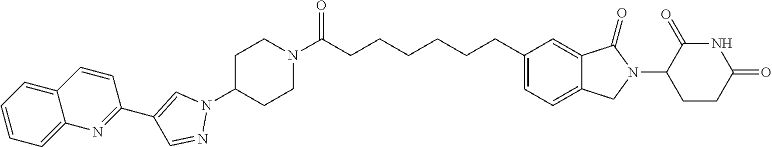 [SMILES: O=C1CCC(N2Cc3ccc(CCCCCCC(=O)N4CCC(n5cc(-c6ccc7ccccc7n6)cn5)CC4)cc3C2=O)C(=O)N1] | 3-(1-oxo-6-(7-oxo-7-(4-(4-(quinolin-2- yl)-1H-pyrazol-1-yl)piperidin-1- yl)heptyl)isoindolin-2-yl)piperidine-2,6- dione |
| GS-099 | 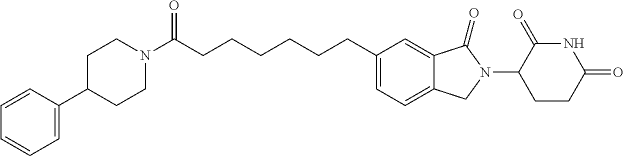 [SMILES: O=C1CCC(N2Cc3ccc(CCCCCCC(=O)N4CCC(c5ccccc5)CC4)cc3C2=O)C(=O)N1] | 3-(1-oxo-6-(7-oxo-7-(4-phenylpiperidin- 1-yl)heptyl)isoindolin-2-yl)piperidine- 2,6-dione |
| GS-100 | 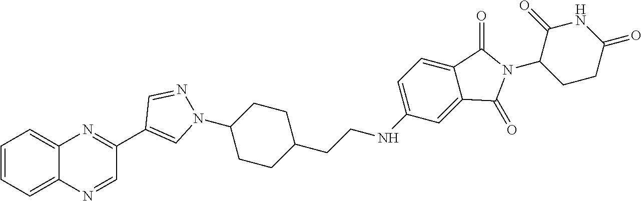 [SMILES: O=C1CCC(N2C(=O)c3ccc(NCCC4CCC(n5cc(-c6cnc7ccccc7n6)cn5)CC4)cc3C2=O)C(=O)N1] | 2-(2,6-dioxopiperidin-3-yl)-5-((2-(4-(4- (quinoxalin-2-yl)-1H-pyrazol-1- yl)cyclohexyl)ethyl)amino)isoindoline- 1,3-dione |
| GS-101 | 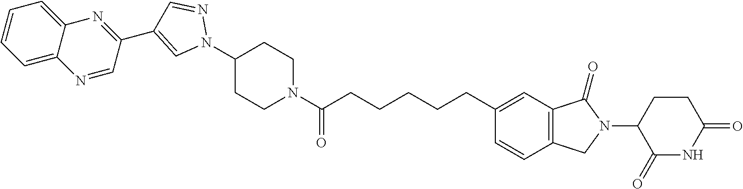 [SMILES: O=C1CCC(N2Cc3ccc(CCCCCC(=O)N4CCC(n5cc(-c6cnc7ccccc7n6)cn5)CC4)cc3C2=O)C(=O)N1] | 3-(1-oxo-6-(6-oxo-6-(4-(4-(quinoxalin-2- yl)-1H-pyrazol-1-yl)piperidin-1- yl)hexyl)isoindolin-2-yl)piperidine-2,6- dione |
| GS-102 | 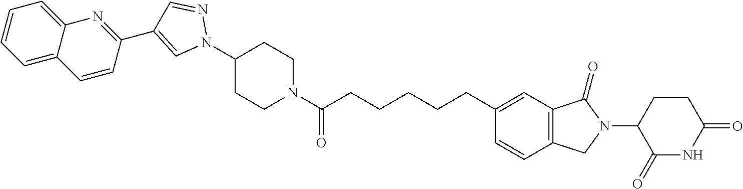 [SMILES: O=C1CCC(N2Cc3ccc(CCCCCC(=O)N4CCC(n5cc(-c6ccc7ccccc7n6)cn5)CC4)cc3C2=O)C(=O)N1] | 3-(1-oxo-6-(6-oxo-6-(4-(4-(quinolin-2- yl)-1H-pyrazol-1-yl)piperidin-1- yl)hexyl)isoindolin-2-yl)piperidine-2,6- dione |
| GS-103 | 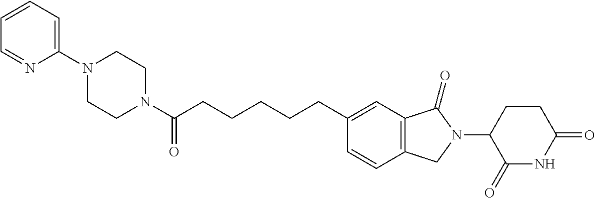 [SMILES: O=C1CCC(N2Cc3ccc(CCCCCC(=O)N4CCN(c5ccccn5)CC4)cc3C2=O)C(=O)N1] | 3-(1-oxo-6-(6-oxo-6-(4-(pyridin-2- yl)piperazin-1-yl)hexyl)isoindolin-2- yl)piperidine-2,6-dione |
| GS-104 | 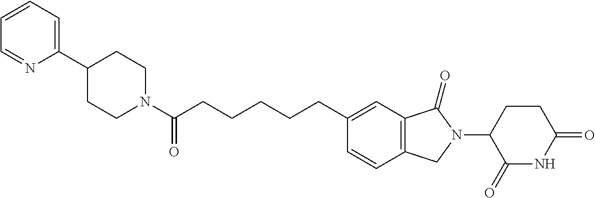 [SMILES: O=C1CCC(N2Cc3ccc(CCCCCC(=O)N4CCC(c5ccccn5)CC4)cc3C2=O)C(=O)N1] | 3-(1-oxo-6-(6-oxo-6-(4-(pyridin-2- yl)piperidin-1-yl)hexyl)isoindolin-2- yl)piperidine-2,6-dione |
| GS-105 | 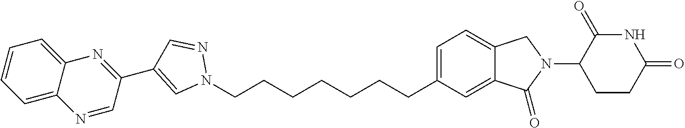 [SMILES: O=C1CCC(N2Cc3ccc(CCCCCCCn4cc(-c5cnc6ccccc6n5)cn4)cc3C2=O)C(=O)N1] | 3-(1-oxo-6-(7-(4-(quinoxalin-2-yl)-1H- pyrazol-1-yl)heptyl)isoindolin-2- yl)piperidine-2,6-dione |
| GS-106 | 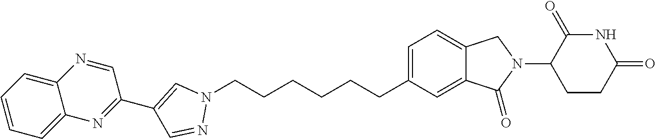 [SMILES: O=C1CCC(N2Cc3ccc(CCCCCCn4cc(-c5cnc6ccccc6n5)cn4)cc3C2=O)C(=O)N1] | 3-(1-oxo-6-(6-(4-(quinoxalin-2-yl)-1H- pyrazol-1-yl)hexyl)isoindolin-2- yl)piperidine-2,6-dione |
| GS-107 | 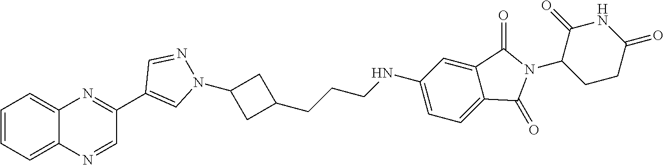 [SMILES: O=C1CCC(N2C(=O)c3ccc(NCCCC4CC(n5cc(-c6cnc7ccccc7n6)cn5)C4)cc3C2=O)C(=O)N1] | 2-(2,6-dioxopiperidin-3-yl)-5-((3-(3-(4- (quinoxalin-2-yl)-1H-pyrazol-1- yl)cyclobutyl)propyl)amino)isoindoline- 1,3-dione |
| GS-108 | 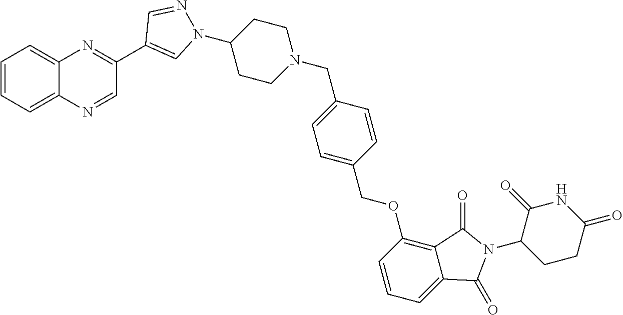 [SMILES: O=C1CCC(N2C(=O)c3cccc(OCc4ccc(CN5CCC(n6cc(-c7cnc8ccccc8n7)cn6)CC5)cc4)c3C2=O)C(=O)N1] | 3-(1-oxo-4-((4-((4-(4-(quinoxalin-2-yl)- 1H-pyrazol-1-yl)piperidin-1- yl)methyl)benzyl)oxy)isoindolin-2- yl)piperidine-2,6-dione |
| GS-109 | 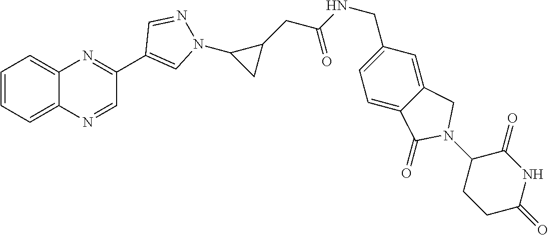 [SMILES: O=C(CC1CC1n1cc(-c2cnc3ccccc3n2)cn1)NCc1ccc2c(c1)CN(C1CCC(=O)NC1=O)C2=O] | N-((2-(2,6-dioxopiperidin-3-yl)-1- oxoisoindolin-5-yl)methyl)-2-(2-(4- (quinoxalin-2-yl)-1H-pyrazol-1- yl)cyclopropyl)acetamide |
| GS-110 | 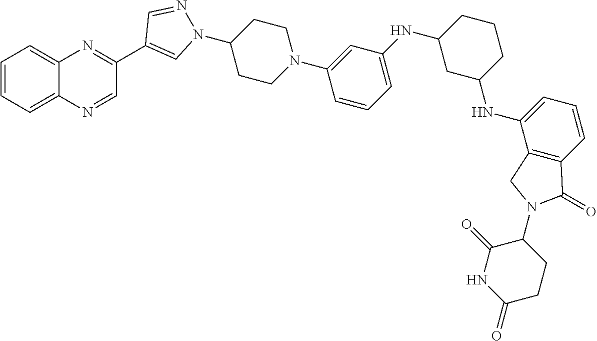 [SMILES: O=C1CCC(N2Cc3c(NC4CCCC(Nc5cccc(N6CCC(n7cc(-c8cnc9ccccc9n8)cn7)CC6)c5)C4)cccc3C2=O)C(=O)N1] | 3-(1-oxo-4-((3-((3-(4-(4-(quinoxalin-2- yl)-1H-pyrazol-1-yl)piperidin-1- yl)phenyl)amino)cyclohexyl)amino)iso- indolin-2-yl)piperidine-2,6-dione |
| GS-111 | 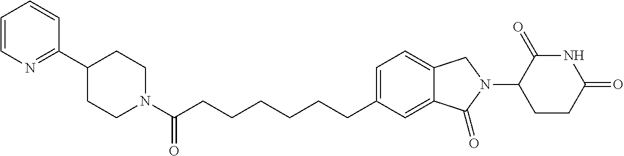 [SMILES: O=C1CCC(N2Cc3ccc(CCCCCCC(=O)N4CCC(c5ccccn5)CC4)cc3C2=O)C(=O)N1] | 3-(1-oxo-6-(7-oxo-7-(4-(pyridin-2- yl)piperidin-1-yl)heptyl)isoindolin-2- yl)piperidine-2,6-dione |
| GS-112 | 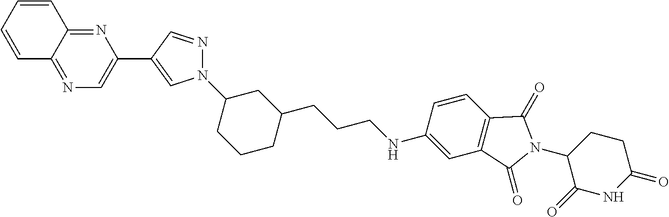 [SMILES: O=C1CCC(N2C(=O)c3ccc(NCCCC4CCCC(n5cc(-c6cnc7ccccc7n6)cn5)C4)cc3C2=O)C(=O)N1] | 2-(2,6-dioxopiperidin-3-yl)-5-((3-(3-(4- (quinoxalin-2-yl)-1H-pyrazol-1- yl)cyclohexyl)propyl)amino)isoindoline- 1,3-dione |
| GS-113 | 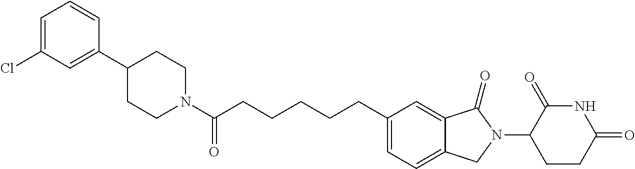 [SMILES: O=C1CCC(N2Cc3ccc(CCCCCC(=O)N4CCC(c5cccc(Cl)c5)CC4)cc3C2=O)C(=O)N1] | 3-(6-(6-(4-(3-chlorophenyl)piperidin-1- yl)-6-oxohexyl)-1-oxoisoindolin-2- yl)piperidine-2,6-dione |
| GS-114 | 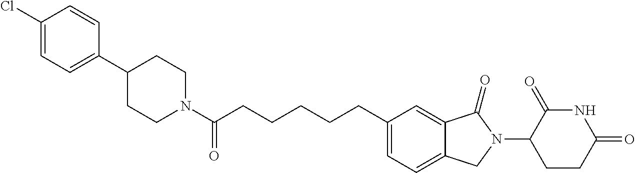 [SMILES: O=C1CCC(N2Cc3ccc(CCCCCC(=O)N4CCC(c5ccc(Cl)cc5)CC4)cc3C2=O)C(=O)N1] | 3-(6-(6-(4-(4-chlorophenyl)piperidin-1- yl)-6-oxohexyl)-1-oxoisoindolin-2- yl)piperidine-2,6-dione |
| GS-115 | 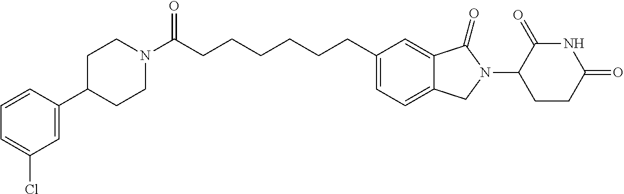 [SMILES: O=C1CCC(N2Cc3ccc(CCCCCCC(=O)N4CCC(c5cccc(Cl)c5)CC4)cc3C2=O)C(=O)N1] | 3-(6-(7-(4-(3-chlorophenyl)piperidin-1- yl)-7-oxoheptyl)-1-oxoisoindolin-2- yl)piperidine-2,6-dione |
| GS-116 | 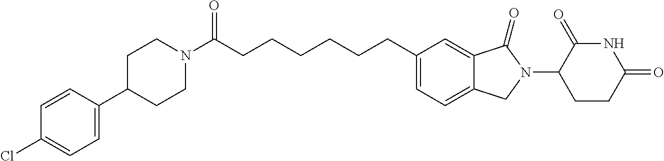 [SMILES: O=C1CCC(N2Cc3ccc(CCCCCCC(=O)N4CCC(c5ccc(Cl)cc5)CC4)cc3C2=O)C(=O)N1] | 3-(6-(7-(4-(4-chlorophenyl)piperidin-1- yl)-7-oxoheptyl)-1-oxoisoindolin-2- yl)piperidine-2,6-dione |
| GS-117 | 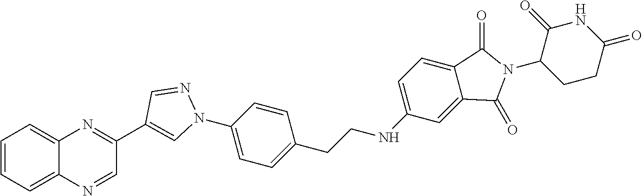 [SMILES: O=C1CCC(N2C(=O)c3ccc(NCCc4ccc(-n5cc(-c6cnc7ccccc7n6)cn5)cc4)cc3C2=O)C(=O)N1] | 2-(2,6-dioxopiperidin-3-yl)-5-((4-(4- (quinoxalin-2-yl)-1H-pyrazol-1- yl)phenethyl)amino)isoindoline-1,3-dione |
| GS-118 | 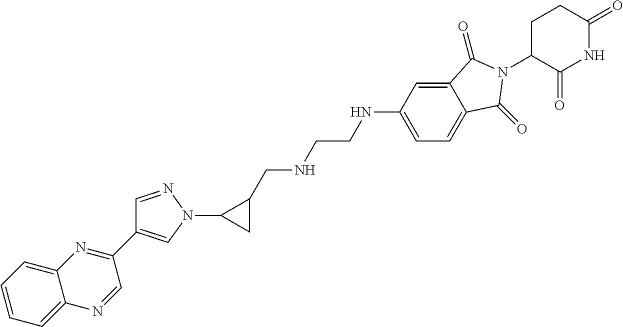 [SMILES: O=C1CCC(N2C(=O)c3ccc(NCCNCC4CC4n4cc(-c5cnc6ccccc6n5)cn4)cc3C2=O)C(=O)N1] | 2-(2,6-dioxopiperidin-3-yl)-5-((2-(((2-(4- (quinoxalin-2-yl)-1H-pyrazol-1- yl)cyclopropyl)methyl)amino)ethyl) amino)isoindoline-1,3-dione |
| GS-119 | 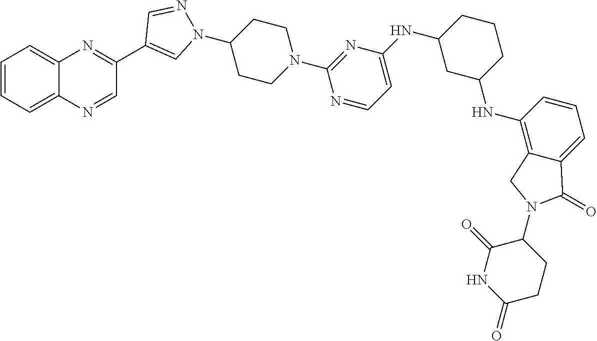 [SMILES: O=C1CCC(N2Cc3c(NC4CCCC(Nc5ccnc(N6CCC(n7cc(-c8cnc9ccccc9n8)cn7)CC6)n5)C4)cccc3C2=O)C(=O)N1] | 3-(1-oxo-4-((3-((2-(4-(4-(quinoxalin-2- yl)-1H-pyrazol-1-yl)piperidin-1- yl)pyrimidin-4- yl)amino)cyclohexyl)amino)isoindolin-2- yl)piperidine-2,6-dione |
| GS-120 | 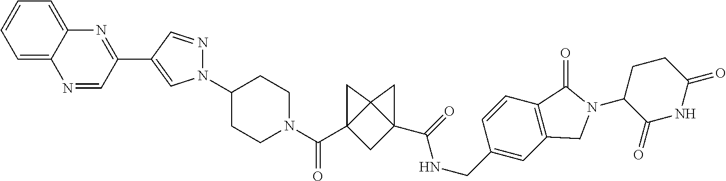 [SMILES: O=C1CCC(N2Cc3cc(CNC(=O)C45CC6(C(=O)N7CCC(n8cc(-c9cnc%10ccccc%10n9)cn8)CC7)CC46C5)ccc3C2=O)C(=O)N1] | N-((2-(2,6-dioxopiperidin-3-yl)-1- oxoisoindolin-5-yl)methyl)-3-(4-(4- (quinoxalin-2-yl)-1H-pyrazol-1- yl)piperidine-1- carbonyl)bicyclo[1.1.1]pentane-1- carboxamide |
| GS-121 | 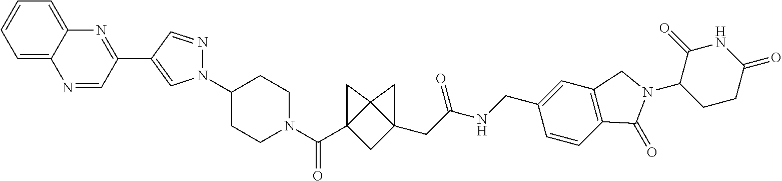 [SMILES: O=C(CC12CC3(C(=O)N4CCC(n5cc(-c6cnc7ccccc7n6)cn5)CC4)CC13C2)NCc1ccc2c(c1)CN(C1CCC(=O)NC1=O)C2=O] | N-((2-(2,6-dioxopiperidin-3-yl)-1- oxoisoindolin-5-yl)methyl)-2-(3-(4-(4- (quinoxalin-2-yl)-1H-pyrazol-1- yl)piperidine-1- carbonyl)bicyclo[1.1.1]pentan-1- yl)acetamide |
| GS-122 | 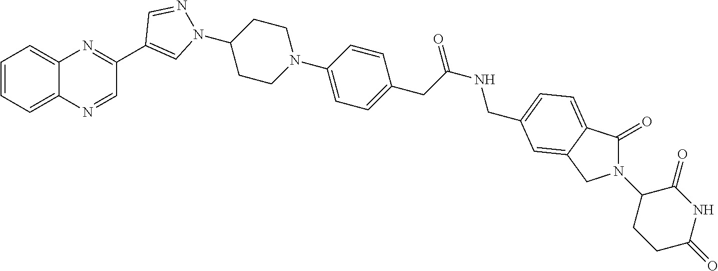 [SMILES: O=C(Cc1ccc(N2CCC(n3cc(-c4cnc5ccccc5n4)cn3)CC2)cc1)NCc1ccc2c(c1)CN(C1CCC(=O)NC1=O)C2=O] | N-((2-(2,6-dioxopiperidin-3-yl)-1- oxoisoindolin-5-yl)methyl)-2-(4-(4-(4- (quinoxalin-2-yl)-1H-pyrazol-1- yl)piperidin-1-yl)phenyl)acetamide |
| GS-123 | 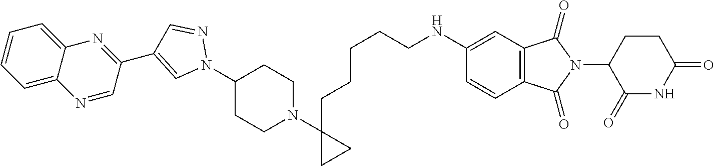 [SMILES: O=C1CCC(N2C(=O)c3ccc(NCCCCCC4(N5CCC(n6cc(-c7cnc8ccccc8n7)cn6)CC5)CC4)cc3C2=O)C(=O)N1] | 2-(2,6-dioxopiperidin-3-yl)-5-((5-(1-(4- (4-(quinoxalin-2-yl)-1H-pyrazol-1- yl)piperidin-1- yl)cyclopropyl)pentyl)amino)isoindoline- 1,3-dione |
| GS-124 | 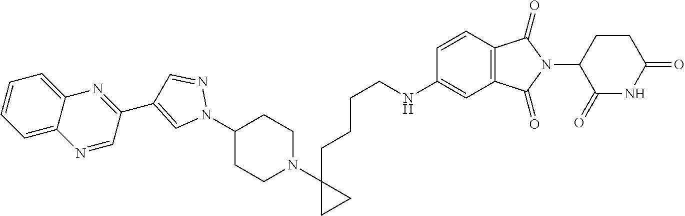 [SMILES: O=C1CCC(N2C(=O)c3ccc(NCCCCC4(N5CCC(n6cc(-c7cnc8ccccc8n7)cn6)CC5)CC4)cc3C2=O)C(=O)N1] | 2-(2,6-dioxopiperidin-3-yl)-5-((4-(1-(4- (4-(quinoxalin-2-yl)-1H-pyrazol-1- yl)piperidin-1- yl)cyclopropyl)butyl)amino)isoindoline- 1,3-dione |
| GS-125 | 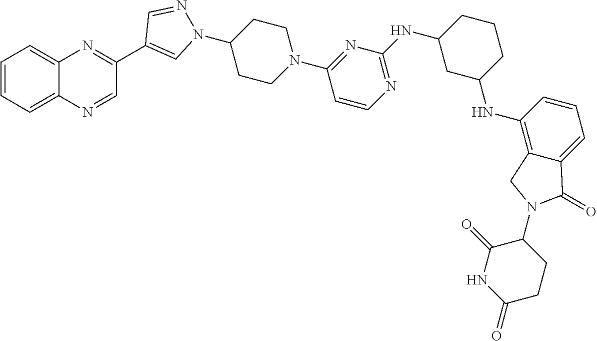 [SMILES: O=C1CCC(N2Cc3c(NC4CCCC(Nc5nccc(N6CCC(n7cc(-c8cnc9ccccc9n8)cn7)CC6)n5)C4)cccc3C2=O)C(=O)N1] | 3-(1-oxo-4-((3-((4-(4-(4-(quinoxalin-2- yl)-1H-pyrazol-1-yl)piperidin-1- yl)pyrimidin-2- yl)amino)cyclohexyl)amino)isoindolin-2- yl)piperidine-2,6-dione |
| GS-126 | 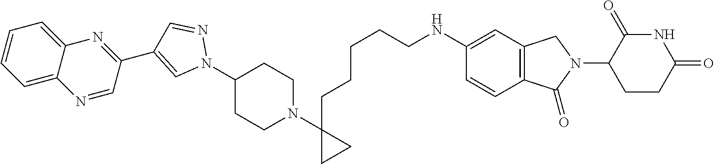 [SMILES: O=C1CCC(N2Cc3cc(NCCCCCC4(N5CCC(n6cc(-c7cnc8ccccc8n7)cn6)CC5)CC4)ccc3C2=O)C(=O)N1] | 3-(1-oxo-5-((5-(1-(4-(4-(quinoxalin-2- yl)-1H-pyrazol-1-yl)piperidin-1- yl)cyclopropyl)pentyl)amino)isoindolin- 2-yl)piperidine-2,6-dione |
| GS-127 | 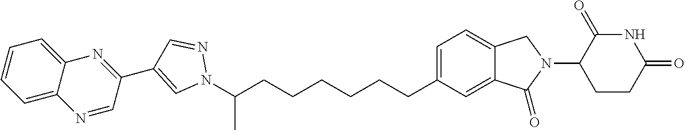 [SMILES: CC(CCCCCCc1ccc2c(c1)C(=O)N(C1CCC(=O)NC1=O)C2)n1cc(-c2cnc3ccccc3n2)cn1] | 3-(1-oxo-6-(7-(4-(quinoxalin-2-yl)-1H- pyrazol-1-yl)octyl)isoindolin-2- yl)piperidine-2,6-dione |
| GS-128 | 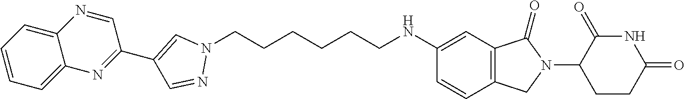 [SMILES: O=C1CCC(N2Cc3ccc(NCCCCCCn4cc(-c5cnc6ccccc6n5)cn4)cc3C2=O)C(=O)N1] | 3-(1-oxo-6-((6-(4-(quinoxalin-2-yl)-1H- pyrazol-1-yl)hexyl)amino)isoindolin-2- yl)piperidine-2,6-dione |
| GS-129 | 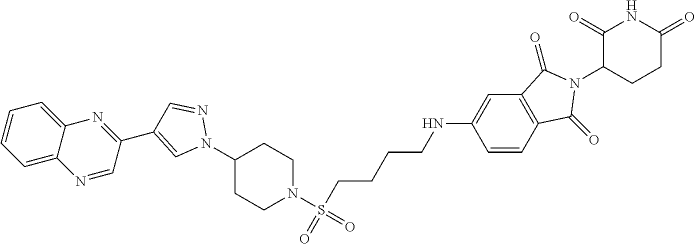 [SMILES: O=C1CCC(N2C(=O)c3ccc(NCCCCS(=O)(=O)N4CCC(n5cc(-c6cnc7ccccc7n6)cn5)CC4)cc3C2=O)C(=O)N1] | 2-(2,6-dioxopiperidin-3-yl)-5-((4-((4-(4- (quinoxalin-2-yl)-1H-pyrazol-1- yl)piperidin-1- yl)sulfonyl)butyl)amino)isoindoline-1,3- dione |
| GS-130 | 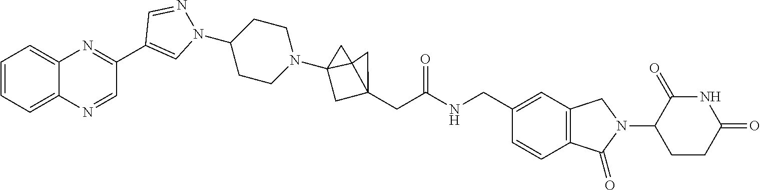 [SMILES: O=C(CC12CC3(N4CCC(n5cc(-c6cnc7ccccc7n6)cn5)CC4)CC13C2)NCc1ccc2c(c1)CN(C1CCC(=O)NC1=O)C2=O] | N-((2-(2,6-dioxopipcridin-3-yl)-1- oxoisoindolin-5-yl)methyl)-2-(3-(4-(4- (quinoxalin-2-yl)-1H-pyrazol-1- yl)piperidin-1-yl)bicyclo[1.1.1]pentan-1- yl)acetamide |
| GS-131 | 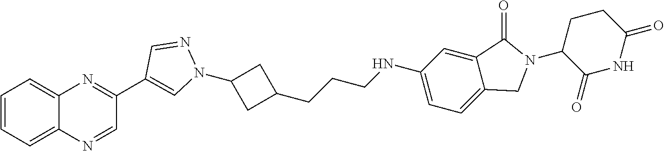 [SMILES: O=C1CCC(N2Cc3ccc(NCCCC4CC(n5cc(-c6cnc7ccccc7n6)cn5)C4)cc3C2=O)C(=O)N1] | 3-(1-oxo-6-((3-(3-(4-(quinoxalin-2-yl)- 1H-pyrazol-1- yl)cyclobutyl)propyl)amino)isoindolin-2- yl)piperidine-2,6-dione |
| GS-132 | 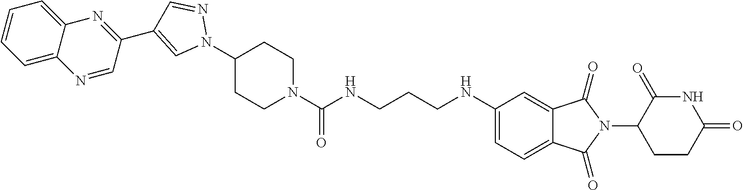 [SMILES: O=C1CCC(N2C(=O)c3ccc(NCCCNC(=O)N4CCC(n5cc(-c6cnc7ccccc7n6)cn5)CC4)cc3C2=O)C(=O)N1] | N-(3-((2-(2,6-dioxopiperidin-3-yl)-1,3- dioxoisoindolin-5-yl)amino)propyl)-4-(4- (quinoxalin-2-yl)-1H-pyrazol-1- yl)piperidine-1-carboxamide |
| GS-133 | 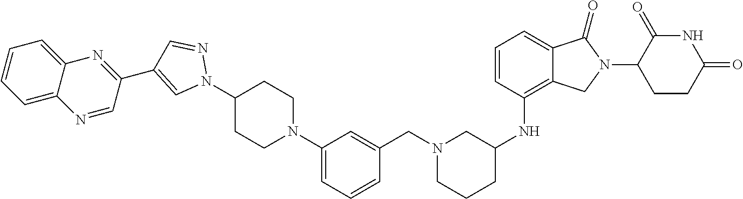 [SMILES: O=C1CCC(N2Cc3c(NC4CCCN(Cc5cccc(N6CCC(n7cc(-c8cnc9ccccc9n8)cn7)CC6)c5)C4)cccc3C2=O)C(=O)N1] | 3-(1-oxo-4-((1-(3-(4-(4-(quinoxalin-2- yl)-1H-pyrazol-1-yl)piperidin-1- yl)benzyl)piperidin-3- yl)amino)isoindolin-2-yl)piperidine-2,6- dione |
| GS-134 | 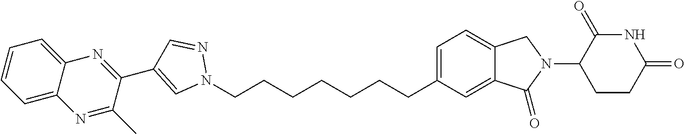 [SMILES: Cc1nc2ccccc2nc1-c1cnn(CCCCCCCc2ccc3c(c2)C(=O)N(C2CCC(=O)NC2=O)C3)c1] | 3-(6-(7-(4-(3-methylquinoxalin-2-yl)-1H- pyrazol-1-yl)heptyl)-1-oxoisoindolin-2- yl)piperidine-2,6-dione |
| GS-135 | 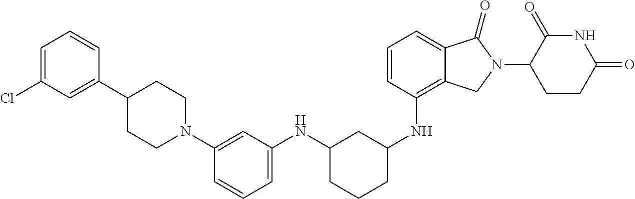 [SMILES: O=C1CCC(N2Cc3c(NC4CCCC(Nc5cccc(N6CCC(c7cccc(Cl)c7)CC6)c5)C4)cccc3C2=O)C(=O)N1] | 3-(4-((3-((3-(4-(3- chlorophenyl)piperidin-1- yl)phenyl)amino)cyclohexyl)amino)-1- oxoisoindolin-2-yl)piperidine-2,6-dione |
| GS-136 | 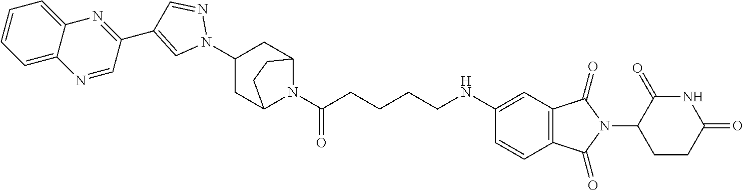 [SMILES: O=C1CCC(N2C(=O)c3ccc(NCCCCC(=O)N4C5CCC4CC(n4cc(-c6cnc7ccccc7n6)cn4)C5)cc3C2=O)C(=O)N1] | 2-(2,6-dioxopiperidin-3-yl)-5-((5-oxo-5- (3-(4-(quinoxalin-2-yl)-1H-pyrazol-1-yl)- 8-azabicyclo[3.2.1]octan-8- yl)pentyl)amino)isoindoline-1,3-dione |
| GS-137 | 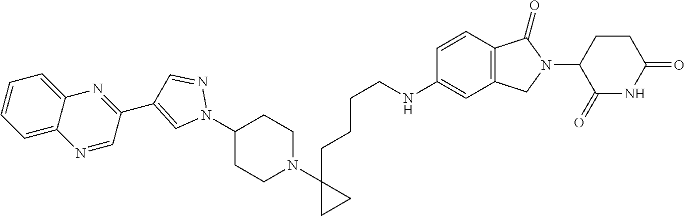 [SMILES: O=C1CCC(N2Cc3cc(NCCCCC4(N5CCC(n6cc(-c7cnc8ccccc8n7)cn6)CC5)CC4)ccc3C2=O)C(=O)N1] | 3-(1-oxo-5-((4-(1-(4-(4-(quinoxalin-2- yl)-1H-pyrazol-1-yl)piperidin-1- yl)cyclopropyl)butyl)amino)isoindolin-2- yl)piperidine-2,6-dione |
| GS-138 | 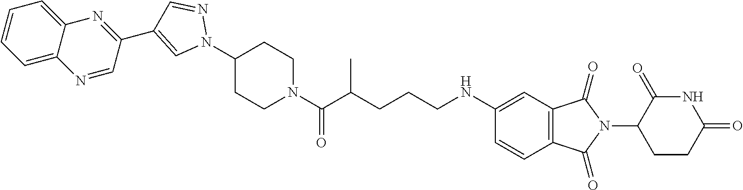 [SMILES: CC(CCCNc1ccc2c(c1)C(=O)N(C1CCC(=O)NC1=O)C2=O)C(=O)N1CCC(n2cc(-c3cnc4ccccc4n3)cn2)CC1] | 2-(2,6-dioxopiperidin-3-yl)-5-((4-methyl- 5-oxo-5-(4-(4-(quinoxalin-2-yl)-1H- pyrazol-1-yl)piperidin-1- yl)pentyl)amino)isoindoline-1,3-dione |
| GS-139 | 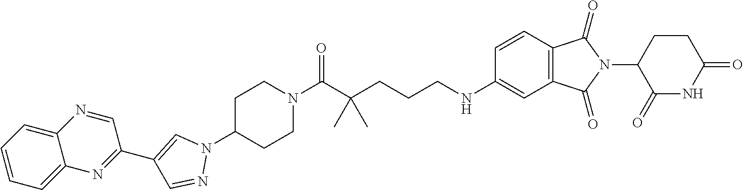 [SMILES: CC(C)(CCCNc1ccc2c(c1)C(=O)N(C1CCC(=O)NC1=O)C2=O)C(=O)N1CCC(n2cc(-c3cnc4ccccc4n3)cn2)CC1] | 5-((4,4-dimethyl-5-oxo-5-(4-(4- (quinoxalin-2-yl)-1H-pyrazol-1- yl)piperidin-1-yl)pentyl)amino)-2-(2,6- dioxopiperidin-3-yl)isoindoline-1,3-dione |
| GS-140 | 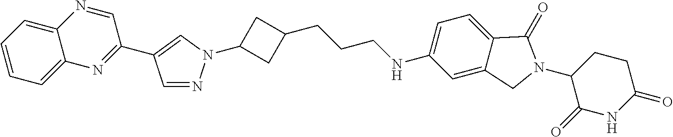 [SMILES: O=C1CCC(N2Cc3cc(NCCCC4CC(n5cc(-c6cnc7ccccc7n6)cn5)C4)ccc3C2=O)C(=O)N1] | 3-(1-oxo-5-((3-(3-(4-(quinoxalin-2-yl)- 1H-pyrazol-1- yl)cyclobutyl)propyl)amino)isoindolin-2- yl)piperidine-2,6-dione |
| GS-141 | 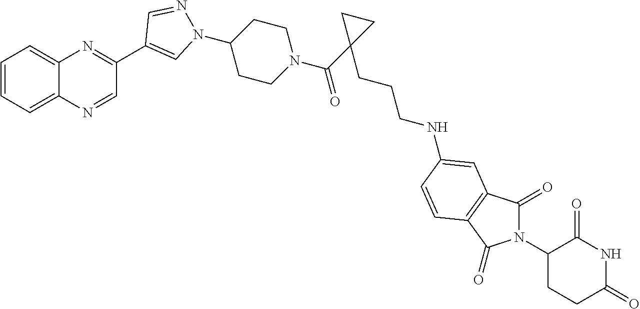 [SMILES: O=C1CCC(N2C(=O)c3ccc(NCCCC4(C(=O)N5CCC(n6cc(-c7cnc8ccccc8n7)cn6)CC5)CC4)cc3C2=O)C(=O)N1] | 2-(2,6-dioxopiperidin-3-yl)-5-((3-(1-(4- (4-(quinoxalin-2-yl)-1H-pyrazol-1- yl)piperidine-1- carbonyl)cyclopropyl)propyl)amino)iso- indoline-1,3-dione |
| GS-142 | 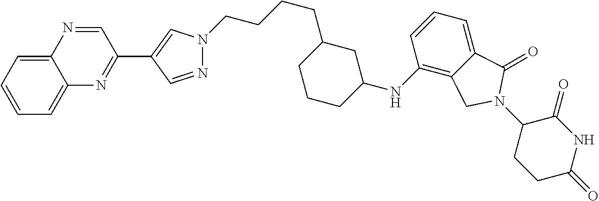 [SMILES: O=C1CCC(N2Cc3c(NC4CCCC(CCCCn5cc(-c6cnc7ccccc7n6)cn5)C4)cccc3C2=O)C(=O)N1] | 3-(1-oxo-4-((3-(4-(4-(quinoxalin-2-yl)- 1H-pyrazol-1- yl)butyl)cyclohexyl)amino)isoindolin-2- yl)piperidine-2,6-dione |
| GS-143 | 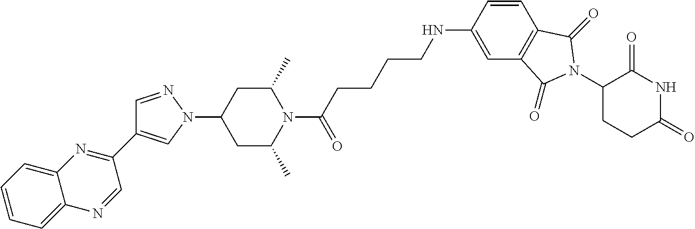 [SMILES: C[C@@H]1CC(n2cc(-c3cnc4ccccc4n3)cn2)C[C@H](C)N1C(=O)CCCCNc1ccc2c(c1)C(=O)N(C1CCC(=O)NC1=O)C2=O] | 5-((5-((2R,6S)-2,6-dimethyl-4-(4- (quinoxalin-2-yl)-1H-pyrazol-1- yl)piperidin-1-yl)-5-oxopentyl)amino)-2- (2,6-dioxopiperidin-3-yl)isoindoline-1,3- dione |
| GS-144 | 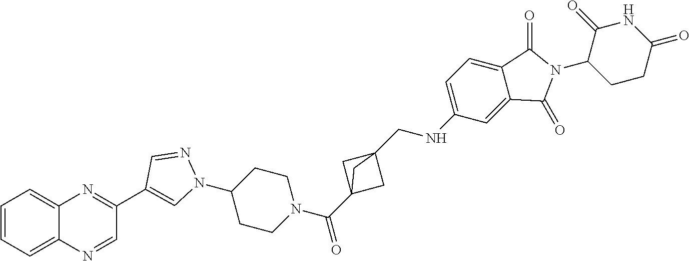 [SMILES: O=C1CCC(N2C(=O)c3ccc(NCC45CC(C(=O)N6CCC(n7cc(-c8cnc9ccccc9n8)cn7)CC6)(C4)C5)cc3C2=O)C(=O)N1] | 2-(2,6-dioxopiperidin-3-yl)-5-(((3-(4-(4- (quinoxalin-2-yl)-1H-pyrazol-1- yl)piperidine-1- carbonyl)bicyclo[1.1.1]pentan-1- yl)methyl)amino)isoindoline-1,3-dione |
| GS-145 | 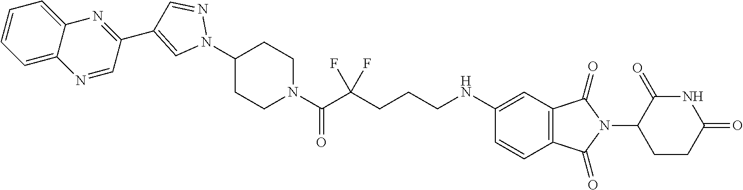 [SMILES: O=C1CCC(N2C(=O)c3ccc(NCCCC(F)(F)C(=O)N4CCC(n5cc(-c6cnc7ccccc7n6)cn5)CC4)cc3C2=O)C(=O)N1] | 5-((4,4-difluoro-5-oxo-5-(4-(4- (quinoxalin-2-yl)-1H-pyrazol-1- yl)piperidin-1-yl)pentyl)amino)-2-(2,6- dioxopiperidin-3-yl)isoindoline-1,3-dione |
| GS-146 | 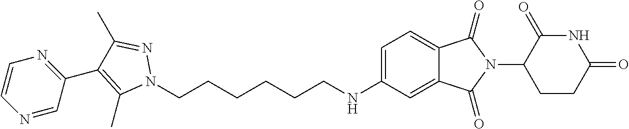 [SMILES: Cc1nn(CCCCCCNc2ccc3c(c2)C(=O)N(C2CCC(=O)NC2=O)C3=O)c(C)c1-c1cnccn1] | 5-((6-(3,5-dimethyl-4-(pyrazin-2-yl)-1H- pyrazol-1-yl)hexyl)amino)-2-(2,6- dioxopiperidin-3-yl)isoindoline-1,3-dione |
| GS-147 | 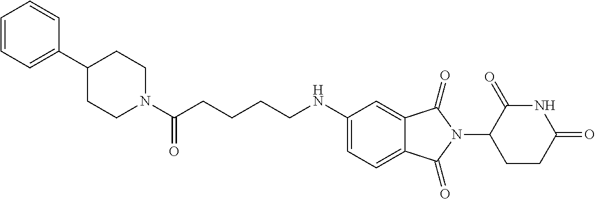 [SMILES: O=C1CCC(N2C(=O)c3ccc(NCCCCC(=O)N4CCC(c5ccccc5)CC4)cc3C2=O)C(=O)N1] | 2-(2,6-dioxopiperidin-3-yl)-5-((5-oxo-5- (4-phenylpiperidin-1- yl)pentyl)amino)isoindoline-1,3-dione |
| GS-148 | 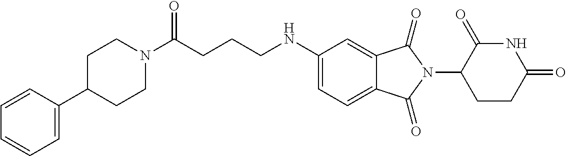 [SMILES: O=C1CCC(N2C(=O)c3ccc(NCCCC(=O)N4CCC(c5ccccc5)CC4)cc3C2=O)C(=O)N1] | 2-(2,6-dioxopiperidin-3-yl)-5-((4-oxo-4- (4-phenylpiperidin-1- yl)butyl)amino)isoindoline-1,3-dione |
| GS-149 | 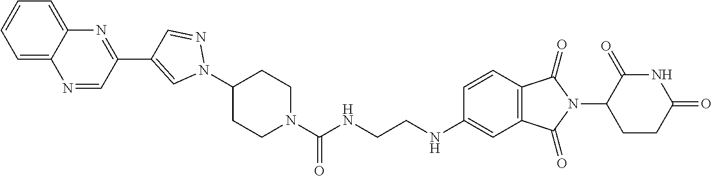 [SMILES: O=C1CCC(N2C(=O)c3ccc(NCCNC(=O)N4CCC(n5cc(-c6cnc7ccccc7n6)cn5)CC4)cc3C2=O)C(=O)N1] | N-(2-((2-(2,6-dioxopiperidin-3-yl)-1,3- dioxoisoindolin-5-yl)amino)ethyl)-4-(4- (quinoxalin-2-yl)-1H-pyrazol-1- yl)piperidine-1-carboxamide |
| GS-150 | 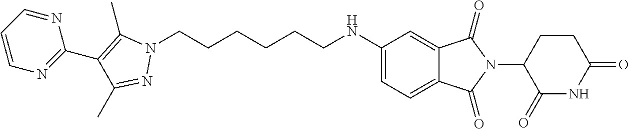 [SMILES: Cc1nn(CCCCCCNc2ccc3c(c2)C(=O)N(C2CCC(=O)NC2=O)C3=O)c(C)c1-c1ncccn1] | 5-((6-(3,5-dimethyl-4-(pyrimidin-2-yl)- 1H-pyrazol-1-yl)hexyl)amino)-2-(2,6- dioxopiperidin-3-yl)isoindoline-1,3-dione |
| GS-151 | 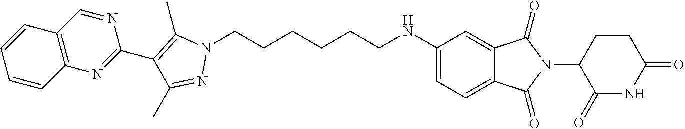 [SMILES: Cc1nn(CCCCCCNc2ccc3c(c2)C(=O)N(C2CCC(=O)NC2=O)C3=O)c(C)c1-c1ncc2ccccc2n1] | 5-((6-(3,5-dimethyl-4-(quinazolin-2-yl)- 1H-pyrazol-1-yl)hexyl)amino)-2-(2,6- dioxopiperidin-3-yl)isoindoline-1,3-dione |
| GS-152 | 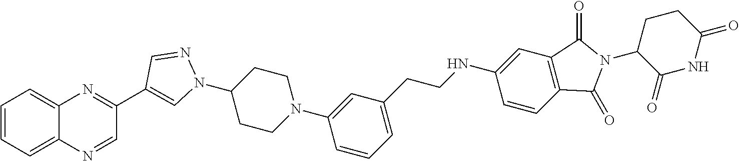 [SMILES: O=C1CCC(N2C(=O)c3ccc(NCCc4cccc(N5CCC(n6cc(-c7cnc8ccccc8n7)cn6)CC5)c4)cc3C2=O)C(=O)N1] | 2-(2,6-dioxopiperidin-3-yl)-5-((3-(4-(4- (quinoxalin-2-yl)-1H-pyrazol-1- yl)piperidin-1- yl)phenethyl)amino)isoindoline-1,3-dione |
| GS-153 | 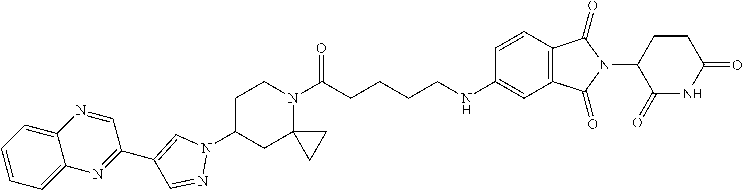 [SMILES: O=C1CCC(N2C(=O)c3ccc(NCCCCC(=O)N4CCC(n5cc(-c6cnc7ccccc7n6)cn5)CC45CC5)cc3C2=O)C(=O)N1] | 2-(2,6-dioxopiperidin-3-yl)-5-((5-oxo-5- (7-(4-(quinoxalin-2-yl)-1H-pyrazol-1-yl)- 4-azaspiro[2.5]octan-4- yl)pentyl)amino)isoindoline-1,3-dione |
| GS-154 | 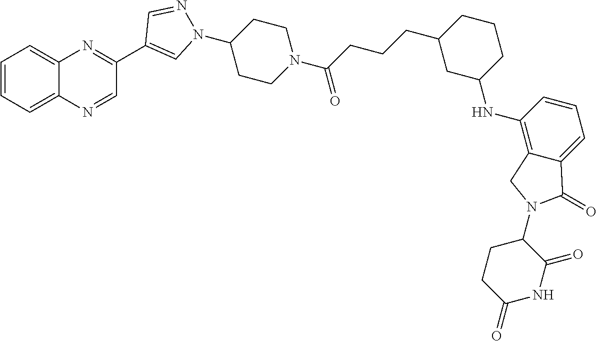 [SMILES: O=C1CCC(N2Cc3c(NC4CCCC(CCCC(=O)N5CCC(n6cc(-c7cnc8ccccc8n7)cn6)CC5)C4)cccc3C2=O)C(=O)N1] | 3-(1-oxo-4-((3-(4-oxo-4-(4-(4- (quinoxalin-2-yl)-1H-pyrazol-1- yl)piperidin-1- yl)butyl)cyclohexyl)amino)isoindolin-2- yl)piperidine-2,6-dione |
| GS-155 | 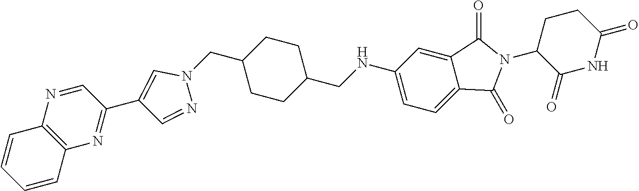 [SMILES: O=C1CCC(N2C(=O)c3ccc(NCC4CCC(Cn5cc(-c6cnc7ccccc7n6)cn5)CC4)cc3C2=O)C(=O)N1] | 2-(2,6-dioxopiperidin-3-yl)-5-(((4-((4- (quinoxalin-2-yl)-1H-pyrazol-1- yl)methyl)cyclohexyl)methyl)amino)iso- indoline-1,3-dione |
| GS-156 | 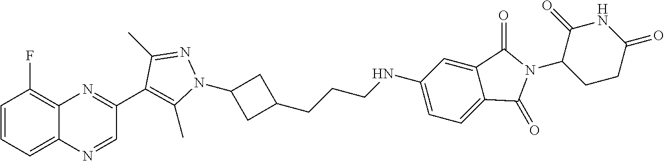 [SMILES: Cc1nn(C2CC(CCCNc3ccc4c(c3)C(=O)N(C3CCC(=O)NC3=O)C4=O)C2)c(C)c1-c1cnc2cccc(F)c2n1] | 2-(2,6-dioxopiperidin-3-yl)-5-((3-(3-(4- (8-fluoroquinoxalin-2-yl)-3,5-dimethyl- 1H-pyrazol-1- yl)cyclobutyl)propyl)amino)isoindoline- 1,3-dione |
| GS-157 | 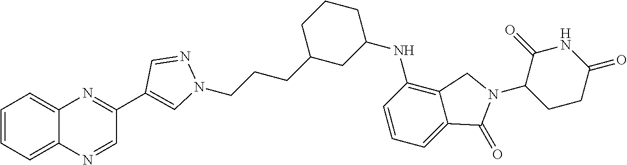 [SMILES: O=C1CCC(N2Cc3c(NC4CCCC(CCCn5cc(-c6cnc7ccccc7n6)cn5)C4)cccc3C2=O)C(=O)N1] | 3-(1-oxo-4-((3-(3-(4-(quinoxalin-2-yl)- 1H-pyrazol-1- yl)propyl)cyclohexyl)amino)isoindolin-2- yl)piperidine-2,6-dione |
| GS-158 | 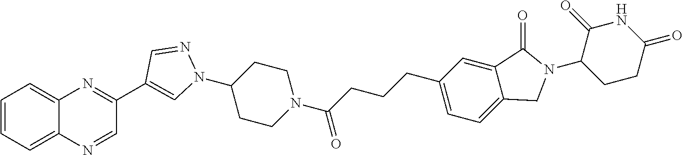 [SMILES: O=C1CCC(N2Cc3ccc(CCCC(=O)N4CCC(n5cc(-c6cnc7ccccc7n6)cn5)CC4)cc3C2=O)C(=O)N1] | 3-(1-oxo-6-(4-oxo-4-(4-(4-(quinoxalin-2- yl)-1H-pyrazol-1-yl)piperidin-1- yl)butyl)isoindolin-2-yl)piperidine-2,6- dione |
| GS-159 | 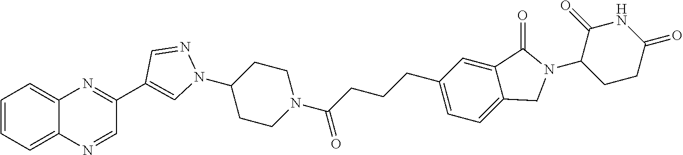 [SMILES: O=C1CCC(N2Cc3ccc(CCCC(=O)N4CCC(n5cc(-c6cnc7ccccc7n6)cn5)CC4)cc3C2=O)C(=O)N1] | 3-(1-oxo-6-(4-oxo-4-(4-(4-(quinolin-2- yl)-1H-pyrazol-1-yl)piperidin-1- yl)butyl)isoindolin-2-yl)piperidine-2,6- dione |
| GS-160 | 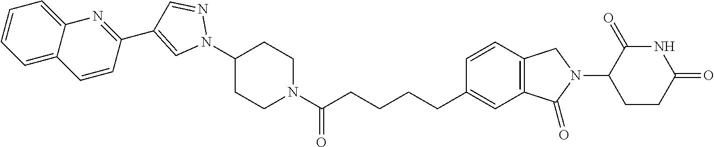 [SMILES: O=C1CCC(N2Cc3ccc(CCCCC(=O)N4CCC(n5cc(-c6ccc7ccccc7n6)cn5)CC4)cc3C2=O)C(=O)N1] | 3-(1-oxo-6-(5-oxo-5-(4-(4-(quinolin-2- yl)-1H-pyrazol-1-yl)piperidin-1- yl)pentyl)isoindolin-2-yl)piperidine-2,6- dione |
| GS-161 | 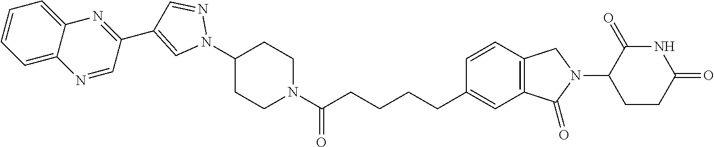 [SMILES: O=C1CCC(N2Cc3ccc(CCCCC(=O)N4CCC(n5cc(-c6cnc7ccccc7n6)cn5)CC4)cc3C2=O)C(=O)N1] | 3-(1-oxo-6-(5-oxo-5-(4-(4-(quinoxalin-2- yl)-1H-pyrazol-1-yl)piperidin-1- yl)pentyl)isoindolin-2-yl)piperidine-2,6- dione |
| GS-162 | 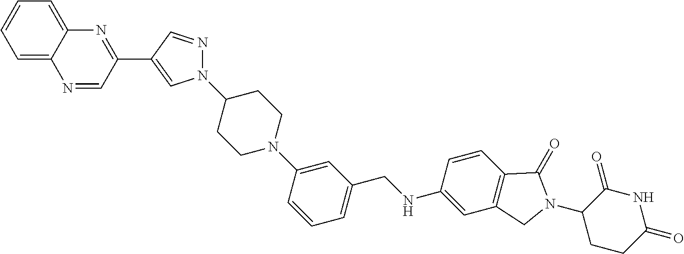 [SMILES: O=C1CCC(N2Cc3cc(NCc4cccc(N5CCC(n6cc(-c7cnc8ccccc8n7)cn6)CC5)c4)ccc3C2=O)C(=O)N1] | 3-(1-oxo-5-((3-(4-(4-(quinoxalin-2-yl)- 1H-pyrazol-1-yl)piperidin-1- yl)benzyl)amino)isoindolin-2- yl)piperidine-2,6-dione |
| GS-163 | 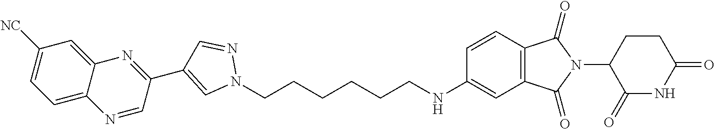 [SMILES: N#Cc1ccc2ncc(-c3cnn(CCCCCCNc4ccc5c(c4)C(=O)N(C4CCC(=O)NC4=O)C5=O)c3)nc2c1] | 3-(1-(6-((2-(2,6-dioxopiperidin-3-yl)-1,3- dioxoisoindolin-5-yl)amino)hexyl)-1H- pyrazol-4-yl)quinoxaline-6-carbonitrile |
| GS-164 | 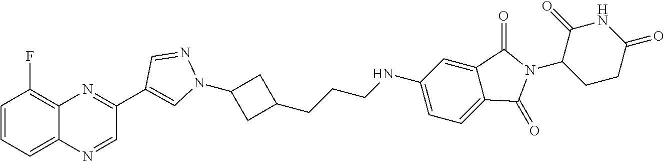 [SMILES: O=C1CCC(N2C(=O)c3ccc(NCCCC4CC(n5cc(-c6cnc7cccc(F)c7n6)cn5)C4)cc3C2=O)C(=O)N1] | 2-(2,6-dioxopiperidin-3-yl)-5-((3-(3-(4- (8-fluoroquinoxalin-2-yl)-1H-pyrazol-1- yl)cyclobutyl)propyl)amino)isoindoline- 1,3-dione |
| GS-165 | 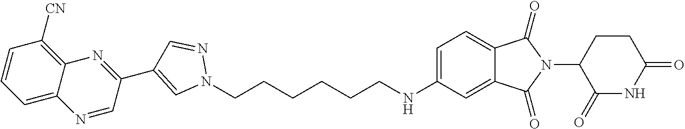 [SMILES: N#Cc1cccc2ncc(-c3cnn(CCCCCCNc4ccc5c(c4)C(=O)N(C4CCC(=O)NC4=O)C5=O)c3)nc12] | 3-(1-(6-((2-(2,6-dioxopiperidin-3-yl)-1,3- dioxoisoindolin-5-yl)amino)hexyl)-1H- pyrazol-4-yl)quinoxaline-5-carbonitrile |
| GS-166 | 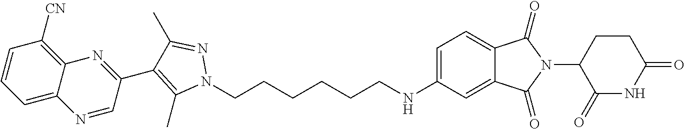 [SMILES: Cc1nn(CCCCCCNc2ccc3c(c2)C(=O)N(C2CCC(=O)NC2=O)C3=O)c(C)c1-c1cnc2cccc(C#N)c2n1] | 3-(1-(6-((2-(2,6-dioxopiperidin-3-yl)-1,3- dioxoisoindolin-5-yl)amino)hexyl)-3,5- dimethyl-1H-pyrazol-4-yl)quinoxaline-5- carbonitrile |
| GS-167 | 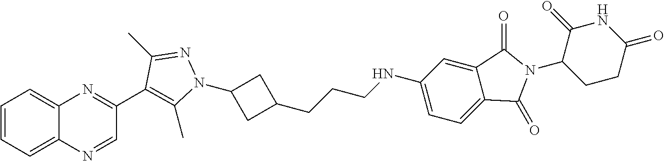 [SMILES: Cc1nn(C2CC(CCCNc3ccc4c(c3)C(=O)N(C3CCC(=O)NC3=O)C4=O)C2)c(C)c1-c1cnc2ccccc2n1] | 5-((3-(3-(3,5-dimethyl-4-(quinoxalin-2- yl)-1H-pyrazol-1- yl)cyclobutyl)propyl)amino)-2-(2,6- dioxopiperidin-3-yl)isoindoline-1,3-dione |
| GS-168 | 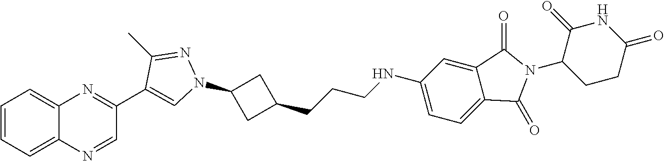 [SMILES: Cc1nn([C@H]2C[C@@H](CCCNc3ccc4c(c3)C(=O)N(C3CCC(=O)NC3=O)C4=O)C2)cc1-c1cnc2ccccc2n1] | 2-(2,6-dioxopiperidin-3-yl)-5-((3-(cis-3- (3-methyl-4-(quinoxalin-2-yl)-1H- pyrazol-1- yl)cyclobutyl)propyl)amino)isoindoline- 1,3-dione |
| GS-169 | 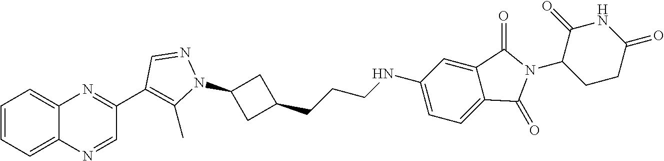 [SMILES: Cc1c(-c2cnc3ccccc3n2)cnn1[C@H]1C[C@@H](CCCNc2ccc3c(c2)C(=O)N(C2CCC(=O)NC2=O)C3=O)C1] | 2-(2,6-dioxopiperidin-3-yl)-5-((3-(cis-3- (5-methyl-4-(quinoxalin-2-yl)-1H- pyrazol-1- yl)cyclobutyl)propyl)amino)isoindoline- 1,3-dione |
| GS-170 | 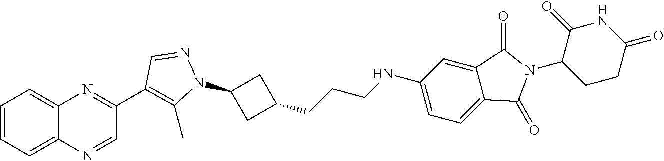 [SMILES: Cc1c(-c2cnc3ccccc3n2)cnn1[C@H]1C[C@H](CCCNc2ccc3c(c2)C(=O)N(C2CCC(=O)NC2=O)C3=O)C1] | 2-(2,6-dioxopiperidin-3-yl)-5-((3-(trans- 3-(5-methyl-4-(quinoxalin-2-yl)-1H- pyrazol-1- yl)cyclobutyl)propyl)amino)isoindoline- 1,3-dione |
| GS-171 | 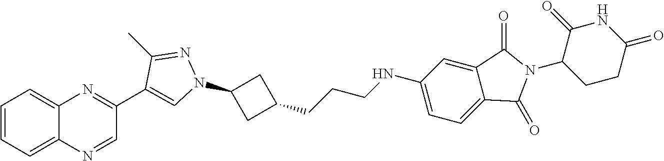 [SMILES: Cc1nn([C@H]2C[C@H](CCCNc3ccc4c(c3)C(=O)N(C3CCC(=O)NC3=O)C4=O)C2)cc1-c1cnc2ccccc2n1] | 2-(2,6-dioxopiperidin-3-yl)-5-((3-(trans- 3-(3-methyl-4-(quinoxalin-2-yl)-1H- pyrazol-1- yl)cyclobutyl)propyl)amino)isoindoline- 1,3-dione |
| GS-172 | 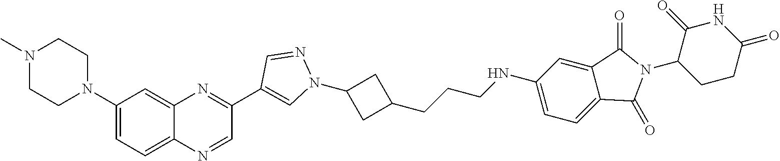 [SMILES: CN1CCN(c2ccc3ncc(-c4cnn(C5CC(CCCNc6ccc7c(c6)C(=O)N(C6CCC(=O)NC6=O)C7=O)C5)c4)nc3c2)CC1] | 2-(2,6-dioxopiperidin-3-yl)-5-((3-(3-(4- (7-(4-methylpiperazin-1-yl)quinoxalin-2- yl)-1H-pyrazol-1- yl)cyclobutyl)propyl)amino)isoindoline- 1,3-dione |
| GS-173 | 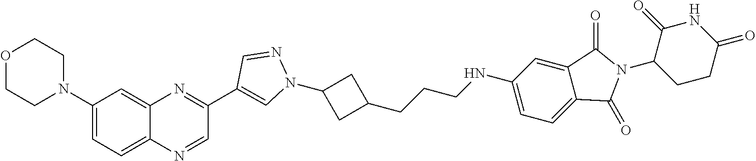 [SMILES: O=C1CCC(N2C(=O)c3ccc(NCCCC4CC(n5cc(-c6cnc7ccc(N8CCOCC8)cc7n6)cn5)C4)cc3C2=O)C(=O)N1] | 2-(2,6-dioxopiperidin-3-yl)-5-((3-(3-(4- (7-morpholinoquinoxalin-2-yl)-1H- pyrazol-1- yl)cyclobutyl)propyl)amino)isoindoline- 1,3-dione |
| GS-174 | 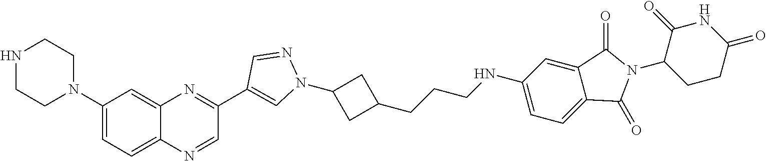 [SMILES: O=C1CCC(N2C(=O)c3ccc(NCCCC4CC(n5cc(-c6cnc7ccc(N8CCNCC8)cc7n6)cn5)C4)cc3C2=O)C(=O)N1] | 2-(2,6-dioxopiperidin-3-yl)-5-((3-(3-(4- (7-(piperazin-1-yl)quinoxalin-2-yl)-1H- pyrazol-1- yl)cyclobutyl)propyl)amino)isoindoline- 1,3-dione |
| GS-175 | 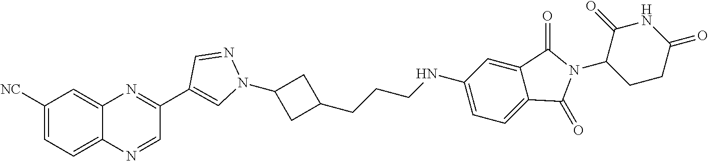 [SMILES: N#Cc1ccc2ncc(-c3cnn(C4CC(CCCNc5ccc6c(c5)C(=O)N(C5CCC(=O)NC5=O)C6=O)C4)c3)nc2c1] | 3-(1-(3-(3-((2-(2,6-dioxopiperidin-3-yl)- 1,3-dioxoisoindolin-5- yl)amino)propyl)cyclobutyl)-1H-pyrazol- 4-yl)quinoxaline-6-carbonitrile |
| GS-176 | 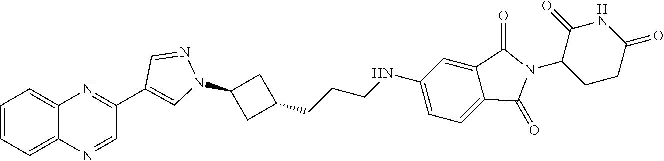 [SMILES: O=C1CCC(N2C(=O)c3ccc(NCCC[C@H]4C[C@H](n5cc(-c6cnc7ccccc7n6)cn5)C4)cc3C2=O)C(=O)N1] | 2-(2,6-dioxopiperidin-3-yl)-5-((3-(trans- 3-(4-(quinoxalin-2-yl)-1H-pyrazol-1- yl)cyclobutyl)propyl)amino)isoindoline- 1,3-dione |
| GS-177 | 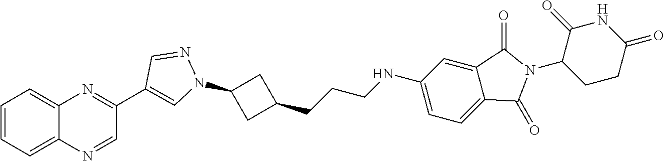 [SMILES: O=C1CCC(N2C(=O)c3ccc(NCCC[C@H]4C[C@@H](n5cc(-c6cnc7ccccc7n6)cn5)C4)cc3C2=O)C(=O)N1] | 2-(2,6-dioxopiperidin-3-yl)-5-((3-(cis-3- (4-(quinoxalin-2-yl)-1H-pyrazol-1- yl)cyclobutyl)propyl)amino)isoindoline- 1,3-dione |
| GS-178 | 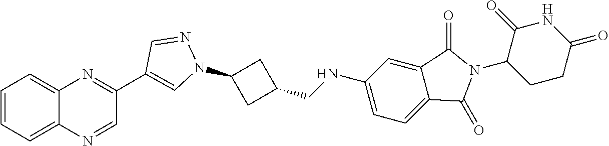 [SMILES: O=C1CCC(N2C(=O)c3ccc(NC[C@H]4C[C@H](n5cc(-c6cnc7ccccc7n6)cn5)C4)cc3C2=O)C(=O)N1] | 2-(2,6-dioxopiperidin-3-yl)-5-(((trans-3- (4-(quinoxalin-2-yl)-1H-pyrazol-1- yl)cyclobutyl)methyl)amino)isoindoline- 1,3-dione |
| GS-179 | 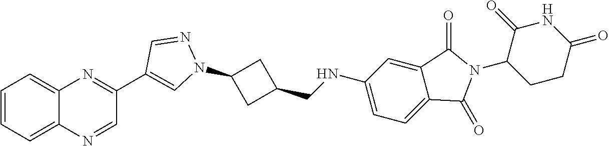 [SMILES: O=C1CCC(N2C(=O)c3ccc(NC[C@H]4C[C@@H](n5cc(-c6cnc7ccccc7n6)cn5)C4)cc3C2=O)C(=O)N1] | 2-(2,6-dioxopiperidin-3-yl)-5-(((cis-3-(4- (quinoxalin-2-yl)-1H-pyrazol-1- yl)cyclobutyl)methyl)amino)isoindoline- 1,3-dione |
| GS-180 | 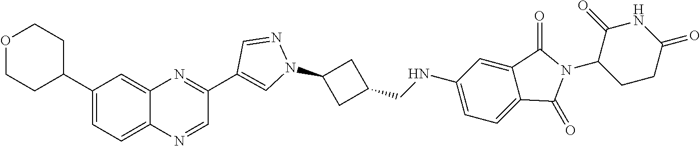 [SMILES: O=C1CCC(N2C(=O)c3ccc(NC[C@H]4C[C@H](n5cc(-c6cnc7ccc(C8CCOCC8)cc7n6)cn5)C4)cc3C2=O)C(=O)N1] | 2-(2,6-dioxopiperidin-3-yl)-5-(((trans-3- (4-(7-(tetrahydro-2H-pyran-4- yl)quinoxalin-2-yl)-1H-pyrazol-1- yl)cyclobutyl)methyl)amino)isoindoline- 1,3-dione |
| GS-181 | 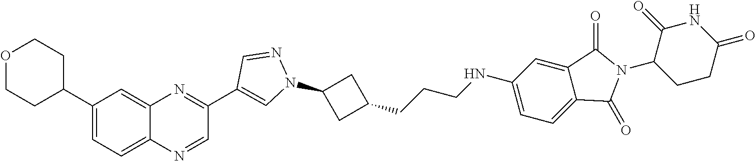 [SMILES: O=C1CCC(N2C(=O)c3ccc(NCCC[C@H]4C[C@H](n5cc(-c6cnc7ccc(C8CCOCC8)cc7n6)cn5)C4)cc3C2=O)C(=O)N1] | 2-(2,6-dioxopiperidin-3-yl)-5-((3-(trans- 3-(4-(7-(tetrahydro-2H-pyran-4- yl)quinoxalin-2-yl)-1H-pyrazol-1- yl)cyclobutyl)propyl)amino)isoindoline- 1,3-dione |
| GS-182 | 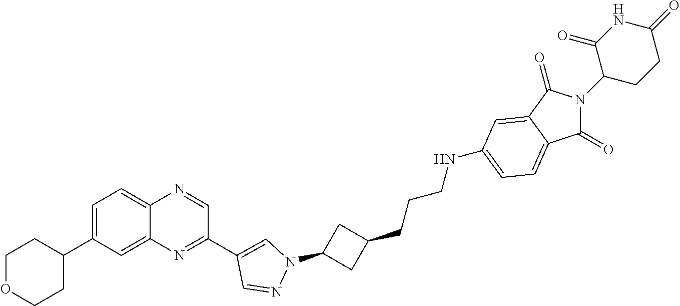 [SMILES: O=C1CCC(N2C(=O)c3ccc(NCCC[C@H]4C[C@@H](n5cc(-c6cnc7ccc(C8CCOCC8)cc7n6)cn5)C4)cc3C2=O)C(=O)N1] | 2-(2,6-dioxopiperidin-3-yl)-5-((3-(cis-3- (4-(7-(tetrahydro-2H-pyran-4- yl)quinoxalin-2-yl)-1H-pyrazol-1- yl)cyclobutyl)propyl)amino)isoindoline- 1,3-dione |
| GS-183 | 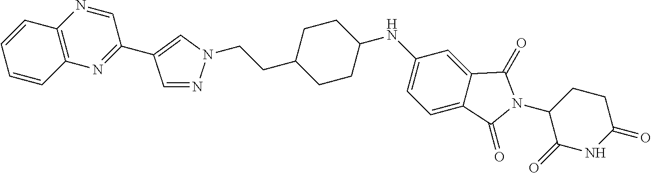 [SMILES: O=C1CCC(N2C(=O)c3ccc(NC4CCC(CCn5cc(-c6cnc7ccccc7n6)cn5)CC4)cc3C2=O)C(=O)N1] | 2-(2,6-dioxopiperidin-3-yl)-5-((4-(2-(4- (quinoxalin-2-yl)-1H-pyrazol-1- soyl)ethyl)cyclohexyl)amino)isoindoline- 1,3-dione |
| GS-184 | 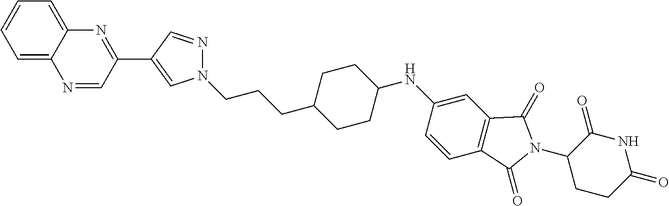 [SMILES: O=C1CCC(N2C(=O)c3ccc(NC4CCC(CCCn5cc(-c6cnc7ccccc7n6)cn5)CC4)cc3C2=O)C(=O)N1] | 2-(2,6-dioxopiperidin-3-yl)-5-((4-(3-(4- (quinoxalin-2-yl)-1H-pyrazol-1- yl)propyl)cyclohexyl)amino)isoindoline- 1,3-dione |
| GS-185 | 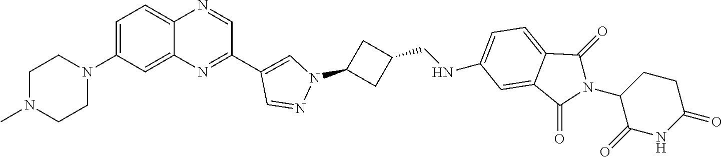 [SMILES: CN1CCN(c2ccc3ncc(-c4cnn([C@H]5C[C@H](CNc6ccc7c(c6)C(=O)N(C6CCC(=O)NC6=O)C7=O)C5)c4)nc3c2)CC1] | 2-(2,6-dioxopiperidin-3-yl)-5-(((trans-3- (4-(7-(4-methylpiperazin-1- yl)quinoxalin-2-yl)-1H-pyrazol-1- yl)cyclobutyl)methyl)amino)isoindoline- 1,3-dione |
| GS-186 | 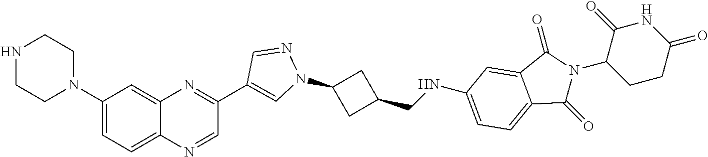 [SMILES: O=C1CCC(N2C(=O)c3ccc(NC[C@H]4C[C@@H](n5cc(-c6cnc7ccc(N8CCNCC8)cc7n6)cn5)C4)cc3C2=O)C(=O)N1] | 2-(2,6-dioxopiperidin-3-yl)-5-(((cis-3-(4- (7-(piperazin-1-yl)quinoxalin-2-yl)-1H- pyrazol-1- yl)cyclobutyl)methyl)amino)isoindoline- 1,3-dione |
| GS-187 | 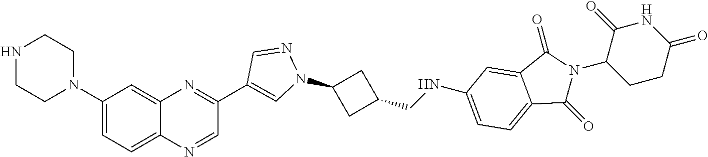 [SMILES: O=C1CCC(N2C(=O)c3ccc(NC[C@H]4C[C@H](n5cc(-c6cnc7ccc(N8CCNCC8)cc7n6)cn5)C4)cc3C2=O)C(=O)N1] | 2-(2,6-dioxopiperidin-3-yl)-5-(((trans-3- (4-(7-(piperazin-1-yl)quinoxalin-2-yl)- 1H-pyrazol-1- yl)cyclobutyl)methyl)amino)isoindoline- 1,3-dione |
| GS-188 | 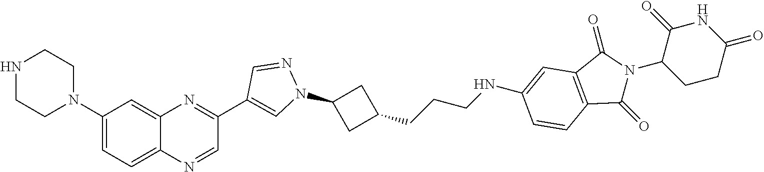 [SMILES: O=C1CCC(N2C(=O)c3ccc(NCCC[C@H]4C[C@H](n5cc(-c6cnc7ccc(N8CCNCC8)cc7n6)cn5)C4)cc3C2=O)C(=O)N1] | 2-(2,6-dioxopiperidin-3-yl)-5-((3-(trans- 3-(4-(7-morpholinoquinoxalin-2-yl)-1H- pyrazol-1- yl)cyclobutyl)propyl)amino)isoindoline- 1,3-dione |
| GS-189 | 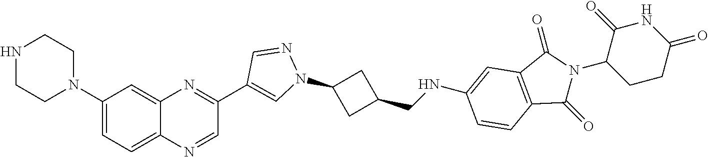 [SMILES: O=C1CCC(N2C(=O)c3ccc(NC[C@H]4C[C@@H](n5cc(-c6cnc7ccc(N8CCNCC8)cc7n6)cn5)C4)cc3C2=O)C(=O)N1] | 2-(2,6-dioxopiperidin-3-yl)-5-(((cis-3-(4- (7-morpholinoquinoxalin-2-yl)-1H- pyrazol-1- yl)cyclobutyl)methyl)amino)isoindoline- 1,3-dione |
| GS-190 | 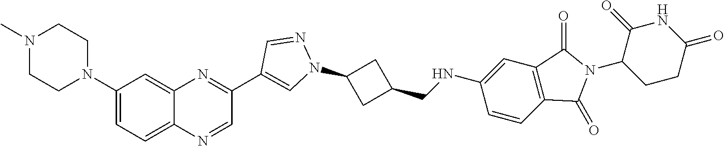 [SMILES: CN1CCN(c2ccc3ncc(-c4cnn([C@H]5C[C@@H](CNc6ccc7c(c6)C(=O)N(C6CCC(=O)NC6=O)C7=O)C5)c4)nc3c2)CC1] | 2-(2,6-dioxopiperidin-3-yl)-5-(((cis-3-(4- (7-(4-methylpiperazin-1-yl)quinoxalin-2- yl)-1H-pyrazol-1- yl)cyclobutyl)methyl)amino)isoindoline- 1,3-dione |
| GS-191 | 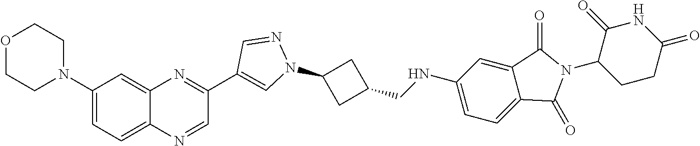 [SMILES: O=C1CCC(N2C(=O)c3ccc(NC[C@H]4C[C@H](n5cc(-c6cnc7ccc(N8CCOCC8)cc7n6)cn5)C4)cc3C2=O)C(=O)N1] | 2-(2,6-dioxopiperidin-3-yl)-5-(((trans-3- (4-(7-morpholinoquinoxalin-2-yl)-1H- pyrazol-1- yl)cyclobutyl)methyl)amino)isoindoline- 1,3-dione |
| GS-192 | 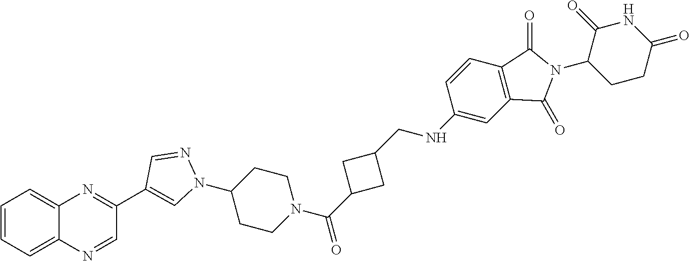 [SMILES: O=C1CCC(N2C(=O)c3ccc(NCC4CC(C(=O)N5CCC(n6cc(-c7cnc8ccccc8n7)cn6)CC5)C4)cc3C2=O)C(=O)N1] | 2-(2,6-dioxopiperidin-3-yl)-5-(((3-(4-(4- (quinoxalin-2-yl)-1H-pyrazol-1- yl)piperidine-1- carbonyl)cyclobutyl)methyl)amino)iso- indoline-1,3-dione |
| GS-193 | 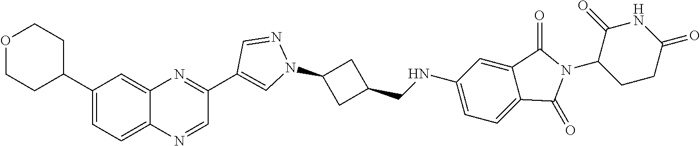 [SMILES: O=C1CCC(N2C(=O)c3ccc(NC[C@H]4C[C@@H](n5cc(-c6cnc7ccc(C8CCOCC8)cc7n6)cn5)C4)cc3C2=O)C(=O)N1] | 2-(2,6-dioxopiperidin-3-yl)-5-(((cis-3-(4- (7-(tetrahydro-2H-pyran-4-yl)quinoxalin- 2-yl)-1H-pyrazol-1- yl)cyclobutyl)methyl)amino)isoindoline- 1,3-dione |
| GS-194 | 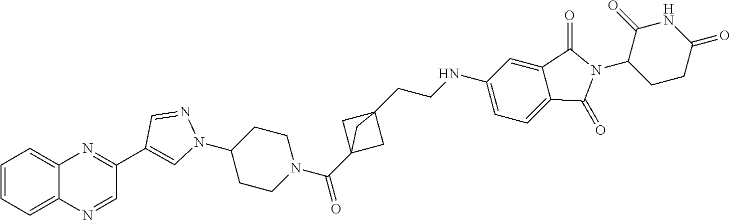 [SMILES: O=C1CCC(N2C(=O)c3ccc(NCCC45CC(C(=O)N6CCC(n7cc(-c8cnc9ccccc9n8)cn7)CC6)(C4)C5)cc3C2=O)C(=O)N1] | 2-(2,6-dioxopiperidin-3-yl)-5-((2-(3-(4- (4-(quinoxalin-2-yl)-1H-pyrazol-1- yl)piperidine-1- carbonyl)bicyclo[1.1.1]pentan-1- yl)ethyl)amino)isoindoline-1,3-dione |
| GS-195 | 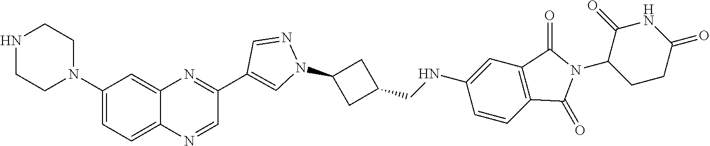 [SMILES: O=C1CCC(N2C(=O)c3ccc(NC[C@H]4C[C@H](n5cc(-c6cnc7ccc(N8CCNCC8)cc7n6)cn5)C4)cc3C2=O)C(=O)N1] | 2-(2,6-dioxopiperidin-3-yl)-5-((3-(trans- 3-(4-(7-(piperazin-1-yl)quinoxalin-2-yl)- 1H-pyrazol-1- yl)cyclobutyl)propyl)amino)isoindoline- 1,3-dione |
| GS-196 | 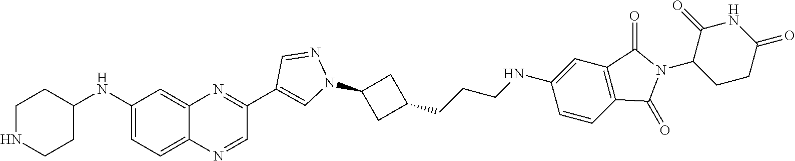 [SMILES: O=C1CCC(N2C(=O)c3ccc(NCCC[C@H]4C[C@H](n5cc(-c6cnc7ccc(NC8CCNCC8)cc7n6)cn5)C4)cc3C2=O)C(=O)N1] | 2-(2,6-dioxopiperidin-3-yl)-5-((3-(trans- 3-(4-(7-(piperidin-4-ylamino)quinoxalin- 2-yl)-1H-pyrazol-1- yl)cyclobutyl)propyl)amino)isoindoline- 1,3-dione |
| GS-197 | 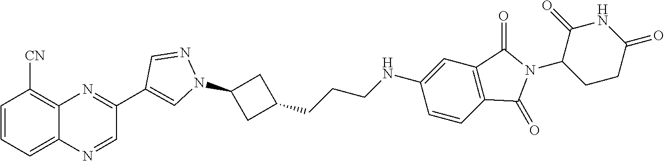 [SMILES: N#Cc1cccc2ncc(-c3cnn([C@H]4C[C@H](CCCNc5ccc6c(c5)C(=O)N(C5CCC(=O)NC5=O)C6=O)C4)c3)nc12] | 3-(1-(trans-3-(3-((2-(2,6-dioxopiperidin- 3-yl)-1,3-dioxoisoindolin-5- yl)amino)propyl)cyclobutyl)-1H-pyrazol- 4-yl)quinoxaline-5-carbonitrile |
| GS-198 | 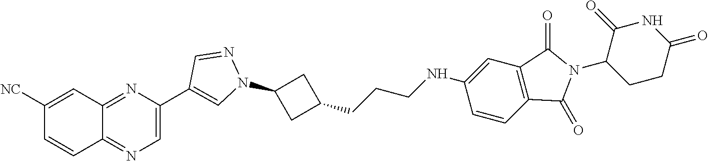 [SMILES: N#Cc1ccc2ncc(-c3cnn([C@H]4C[C@H](CCCNc5ccc6c(c5)C(=O)N(C5CCC(=O)NC5=O)C6=O)C4)c3)nc2c1] | 3-(1-(trans-3-(3-((2-(2,6-dioxopiperidin- 3-yl)-1,3-dioxoisoindolin-5- yl)amino)propyl)cyclobutyl)-1H-pyrazol- 4-yl)quinoxaline-6-carbonitrile |
| GS-199 | 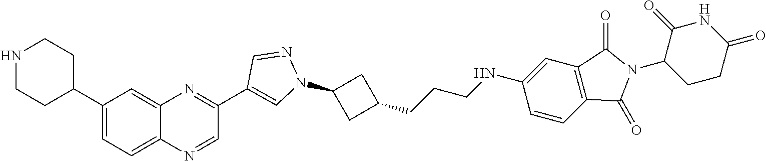 [SMILES: O=C1CCC(N2C(=O)c3ccc(NCCC[C@H]4C[C@H](n5cc(-c6cnc7ccc(C8CCNCC8)cc7n6)cn5)C4)cc3C2=O)C(=O)N1] | 2-(2,6-dioxopiperidin-3-yl)-5-((3-(trans- 3-(4-(7-(piperidin-4-yl)quinoxalin-2-yl)- 1H-pyrazol-1- yl)cyclobutyl)propyl)amino)isoindoline- 1,3-dione |
| GS-200 | 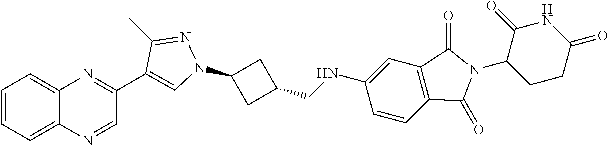 [SMILES: Cc1nn([C@H]2C[C@H](CNc3ccc4c(c3)C(=O)N(C3CCC(=O)NC3=O)C4=O)C2)cc1-c1cnc2ccccc2n1] | 2-(2,6-dioxopiperidin-3-yl)-5-(((trans-3- (3-methyl-4-(quinoxalin-2-yl)-1H- pyrazol-1- yl)cyclobutyl)methyl)amino)isoindoline- 1,3-dione |
| GS-201 | 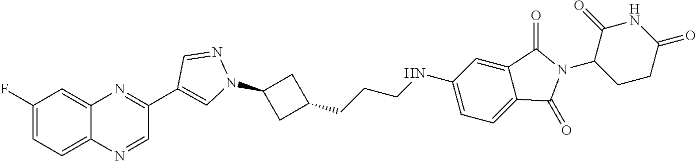 [SMILES: O=C1CCC(N2C(=O)c3ccc(NCCC[C@H]4C[C@H](n5cc(-c6cnc7ccc(F)cc7n6)cn5)C4)cc3C2=O)C(=O)N1] | 2-(2,6-dioxopiperidin-3-yl)-5-((3-(trans- 3-(4-(7-fluoroquinoxalin-2-yl)-1H- pyrazol-1- yl)cyclobutyl)propyl)amino)isoindoline- 1,3-dione |
| GS-202 | 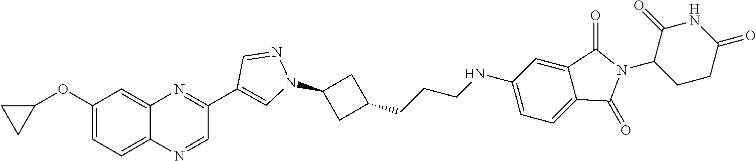 [SMILES: O=C1CCC(N2C(=O)c3ccc(NCCC[C@H]4C[C@H](n5cc(-c6cnc7ccc(OC8CC8)cc7n6)cn5)C4)cc3C2=O)C(=O)N1] | 5-((3-(trans-3-(4-(7- cyclopropoxyquinoxalin-2-yl)-1H- pyrazol-1-yl)cyclobutyl)propyl)amino)-2- (2,6-dioxopiperidin-3-yl)isoindoline-1,3- dione |
| GS-203 | 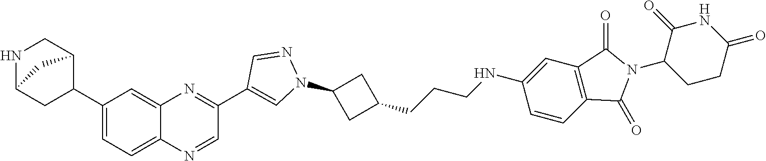 [SMILES: O=C1CCC(N2C(=O)c3ccc(NCCC[C@H]4C[C@H](n5cc(-c6cnc7ccc(C8C[C@@H]9C[C@H]8CN9)cc7n6)cn5)C4)cc3C2=O)C(=O)N1] | 5-((3-(trans-3-(4-(7-((1S,4S)-2,5- diazabicyclo[2.2.1]heptan-2- yl)quinoxalin-2-yl)-1H-pyrazol-1- yl)cyclobutyl)propyl)amino)-2-(2,6- dioxopiperidin-3-yl)isoindoline-1,3-dione |
| GS-204 | 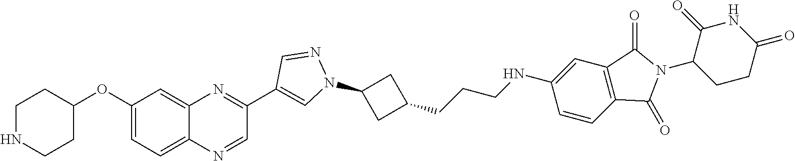 [SMILES: O=C1CCC(N2C(=O)c3ccc(NCCC[C@H]4C[C@H](n5cc(-c6cnc7ccc(OC8CCNCC8)cc7n6)cn5)C4)cc3C2=O)C(=O)N1] | 2-(2,6-dioxopiperidin-3-yl)-5-((3-(trans- 3-(4-(7-(piperidin-4-yloxy)quinoxalin-2- yl)-1H-pyrazol-1- yl)cyclobutyl)propyl)amino)isoindoline- 1,3-dione |
| GS-205 | 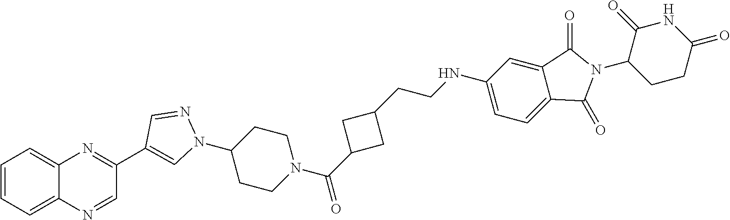 [SMILES: O=C1CCC(N2C(=O)c3ccc(NCCC4CC(C(=O)N5CCC(n6cc(-c7cnc8ccccc8n7)cn6)CC5)C4)cc3C2=O)C(=O)N1] | 2-(2,6-dioxopiperidin-3-yl)-5-((2-(3-(4- (4-(quinoxalin-2-yl)-1H-pyrazol-1- yl)piperidine-1- carbonyl)cyclobutyl)ethyl)amino)iso- indoline-1,3-dione |
| GS-206 | 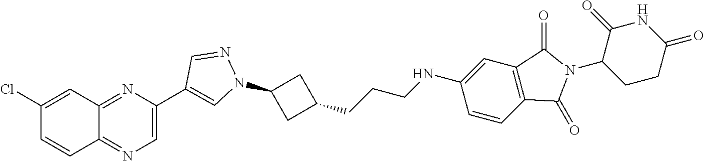 [SMILES: O=C1CCC(N2C(=O)c3ccc(NCCC[C@H]4C[C@H](n5cc(-c6cnc7ccc(Cl)cc7n6)cn5)C4)cc3C2=O)C(=O)N1] | 5-((3-(trans-3-(4-(7-chloroquinoxalin-2- yl)-1H-pyrazol-1- yl)cyclobutyl)propyl)amino)-2-(2,6- dioxopiperidin-3-yl)isoindoline-1,3-dione |
| GS-207 | 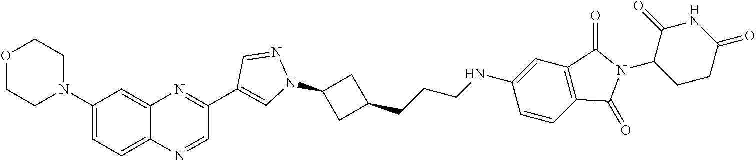 [SMILES: O=C1CCC(N2C(=O)c3ccc(NCCC[C@H]4C[C@@H](n5cc(-c6cnc7ccc(N8CCOCC8)cc7n6)cn5)C4)cc3C2=O)C(=O)N1] | 2-(2,6-dioxopiperidin-3-yl)-5-((3-(cis-3- (4-(7-morpholinoquinoxalin-2-yl)-1H- pyrazol-1- yl)cyclobutyl)propyl)amino)isoindoline- 1,3-dione |
| GS-208 | 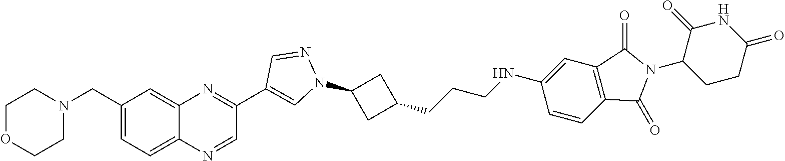 [SMILES: O=C1CCC(N2C(=O)c3ccc(NCCC[C@H]4C[C@H](n5cc(-c6cnc7ccc(CN8CCOCC8)cc7n6)cn5)C4)cc3C2=O)C(=O)N1] | 2-(2,6-dioxopiperidin-3-yl)-5-((3-(trans- 3-(4-(7-(morpholinomethyl)quinoxalin-2- yl)-1H-pyrazol-1- yl)cyclobutyl)propyl)amino)isoindoline- 1,3-dione |
| GS-209 | 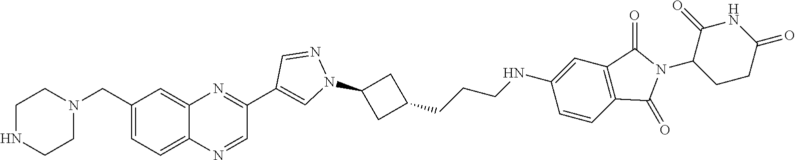 [SMILES: O=C1CCC(N2C(=O)c3ccc(NCCC[C@H]4C[C@H](n5cc(-c6cnc7ccc(CN8CCNCC8)cc7n6)cn5)C4)cc3C2=O)C(=O)N1] | 2-(2,6-dioxopiperidin-3-yl)-5-((3-(trans- 3-(4-(7-(piperazin-1- ylmethyl)quinoxalin-2-yl)-1H-pyrazol-1- yl)cyclobutyl)propyl)amino)isoindoline- 1,3-dione |
| GS-210 | 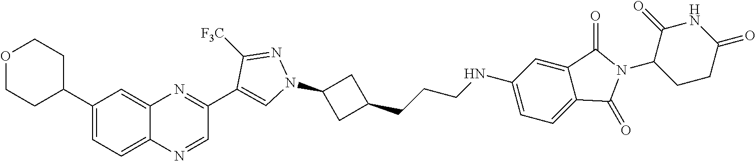 [SMILES: O=C1CCC(N2C(=O)c3ccc(NCCC[C@H]4C[C@@H](n5cc(-c6cnc7ccc(C8CCOCC8)cc7n6)c(C(F)(F)F)n5)C4)cc3C2=O)C(=O)N1] | 2-(2,6-dioxopiperidin-3-yl)-5-((3-(cis-3- (4-(7-(tetrahydro-2H-pyran-4- yl)quinoxalin-2-yl)-3-(trifluoromethyl)- 1H-pyrazol-1- yl)cyclobutyl)propyl)amino)isoindoline- 1,3-dione |
| GS-211 | 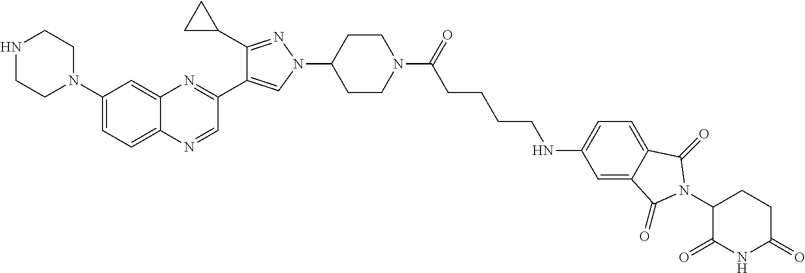 [SMILES: O=C1CCC(N2C(=O)c3ccc(NCCCCC(=O)N4CCC(n5cc(-c6cnc7ccc(N8CCNCC8)cc7n6)c(C6CC6)n5)CC4)cc3C2=O)C(=O)N1] | 5-((5-(4-(3-cyclopropyl-4-(7-(piperazin- 1-yl)quinoxalin-2-yl)-1H-pyrazol-1- yl)piperidin-1-yl)-5-oxopentyl)amino)-2- (2,6-dioxopiperidin-3-yl)isoindoline-1,3- dione |
| GS-212 | 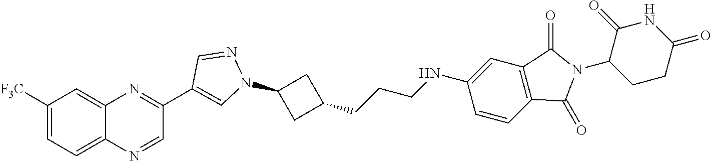 [SMILES: O=C1CCC(N2C(=O)c3ccc(NCCC[C@H]4C[C@H](n5cc(-c6cnc7ccc(C(F)(F)F)cc7n6)cn5)C4)cc3C2=O)C(=O)N1] | 2-(2,6-dioxopiperidin-3-yl)-5-((3-(trans- 3-(4-(7-(trifluoromethyl)quinoxalin-2-yl)- 1H-pyrazol-1- yl)cyclobutyl)propyl)amino)isoindoline- 1,3-dione |
| GS-213 | 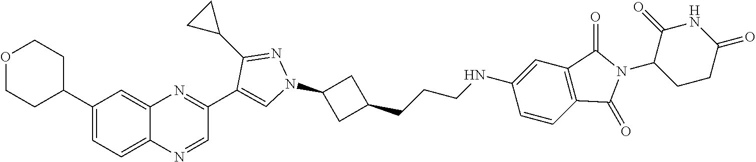 [SMILES: O=C1CCC(N2C(=O)c3ccc(NCCC[C@H]4C[C@@H](n5cc(-c6cnc7ccc(C8CCOCC8)cc7n6)c(C6CC6)n5)C4)cc3C2=O)C(=O)N1] | 5-((3-(cis-3-(3-cyclopropyl-4-(7- (tetrahydro-2H-pyran-4-yl)quinoxalin-2- yl)-1H-pyrazol-1- yl)cyclobutyl)propyl)amino)-2-(2,6- dioxopiperidin-3-yl)isoindoline-1,3-dione |
| GS-214 | 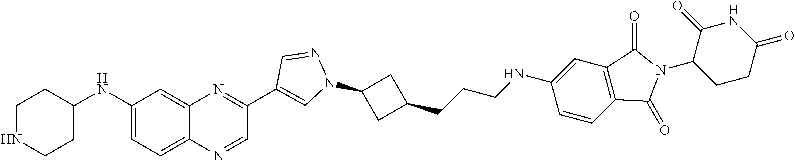 [SMILES: O=C1CCC(N2C(=O)c3ccc(NCCC[C@H]4C[C@@H](n5cc(-c6cnc7ccc(NC8CCNCC8)cc7n6)cn5)C4)cc3C2=O)C(=O)N1] | 2-(2,6-dioxopiperidin-3-yl)-5-((3-(cis-3- (4-(7-(piperidin-4-ylamino)quinoxalin-2- yl)-1H-pyrazol-1- yl)cyclobutyl)propyl)amino)isoindoline- 1,3-dione |
| GS-215 | 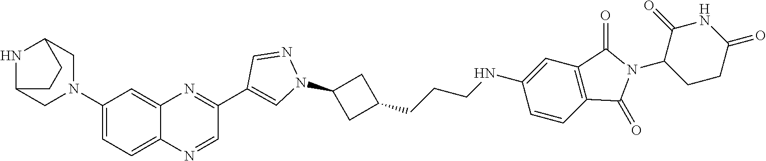 [SMILES: O=C1CCC(N2C(=O)c3ccc(NCCC[C@H]4C[C@H](n5cc(-c6cnc7ccc(N8CC9CCC(C8)N9)cc7n6)cn5)C4)cc3C2=O)C(=O)N1] | 5-((3-(trans-3-(4-(7-(3,8- diazabicyclo[3.2.1]octan-3-yl)quinoxalin- 2-yl)-1H-pyrazol-1- yl)cyclobutyl)propyl)amino)-2-(2,6- dioxopiperidin-3-yl)isoindoline-1,3-dione |
| GS-216 | 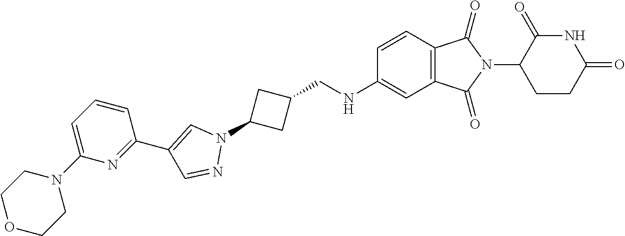 [SMILES: O=C1CCC(N2C(=O)c3ccc(NC[C@H]4C[C@H](n5cc(-c6cccc(N7CCOCC7)n6)cn5)C4)cc3C2=O)C(=O)N1] | 2-(2,6-dioxopiperidin-3-yl)-5-(((trans-3- (4-(6-morpholinopyridin-2-yl)-1H- pyrazol-1- yl)cyclobutyl)methyl)amino)isoindoline- 1,3-dione |
| GS-217 | 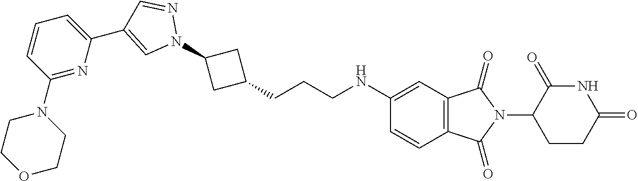 [SMILES: O=C1CCC(N2C(=O)c3ccc(NCCC[C@H]4C[C@H](n5cc(-c6cccc(N7CCOCC7)n6)cn5)C4)cc3C2=O)C(=O)N1] | 2-(2,6-dioxopiperidin-3-yl)-5-((3-(trans- 3-(4-(6-morpholinopyridin-2-yl)-1H- pyrazol-1- yl)cyclobutyl)propyl)amino)isoindoline- 1,3-dione |
| GS-218 | 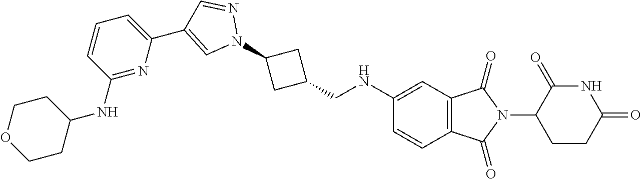 [SMILES: O=C1CCC(N2C(=O)c3ccc(NC[C@H]4C[C@H](n5cc(-c6cccc(NC7CCOCC7)n6)cn5)C4)cc3C2=O)C(=O)N1] | 2-(2,6-dioxopiperidin-3-yl)-5-(((trans-3- (4-(6-((tetrahydro-2H-pyran-4- yl)amino)pyridin-2-yl)-1H-pyrazol-1- yl)cyclobutyl)methyl)amino)isoindoline- 1,3-dione |
| GS-219 | 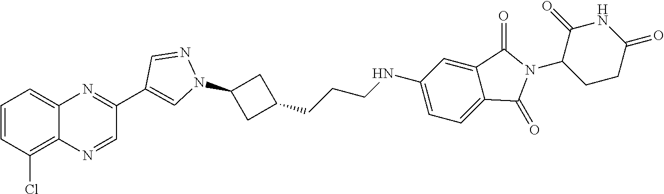 [SMILES: O=C1CCC(N2C(=O)c3ccc(NCCC[C@H]4C[C@H](n5cc(-c6cnc7c(Cl)cccc7n6)cn5)C4)cc3C2=O)C(=O)N1] | 5-((3-(trans-3-(4-(5-chloroquinoxalin-2- yl)-1H-pyrazol-1- yl)cyclobutyl)propyl)amino)-2-(2,6- dioxopiperidin-3-yl)isoindoline-1,3-dione |
| GS-220 | 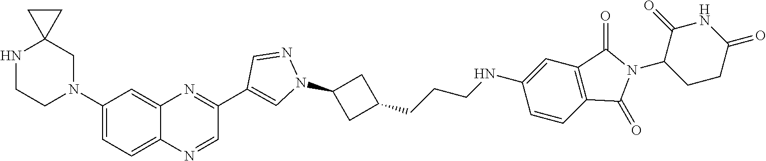 [SMILES: O=C1CCC(N2C(=O)c3ccc(NCCC[C@H]4C[C@H](n5cc(-c6cnc7ccc(N8CCNC9(CC9)C8)cc7n6)cn5)C4)cc3C2=O)C(=O)N1] | 5-((3-(trans-3-(4-(7-(4,7- diazaspiro[2.5]octan-7-yl)quinoxalin-2- yl)-1H-pyrazol-1- yl)cyclobutyl)propyl)amino)-2-(2,6- dioxopiperidin-3-yl)isoindoline-1,3-dione |
| GS-221 | 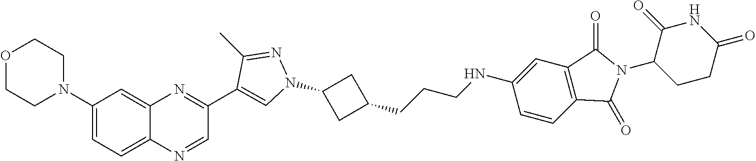 [SMILES: Cc1nn([C@H]2C[C@@H](CCCNc3ccc4c(c3)C(=O)N(C3CCC(=O)NC3=O)C4=O)C2)cc1-c1cnc2ccc(N3CCOCC3)cc2n1] | 2-(2,6-dioxopiperidin-3-yl)-5-((3-(cis-3- (3-methyl-4-(7-morpholinoquinoxalin-2- yl)-1H-pyrazol-1- yl)cyclobutyl)propyl)amino)isoindoline- 1,3-dione |
| GS-222 | 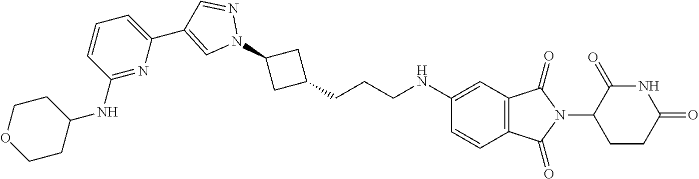 [SMILES: O=C1CCC(N2C(=O)c3ccc(NCCC[C@H]4C[C@H](n5cc(-c6cccc(NC7CCOCC7)n6)cn5)C4)cc3C2=O)C(=O)N1] | 2-(2,6-dioxopiperidin-3-yl)-5-((3-(trans- 3-(4-(6-((tetrahydro-2H-pyran-4- yl)amino)pyridin-2-yl)-1H-pyrazol-1- yl)cyclobutyl)propyl)amino)isoindoline- 1,3-dione |
| GS-223 | 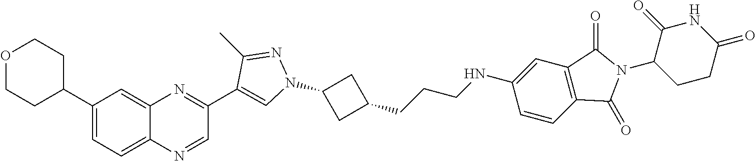 [SMILES: Cc1nn([C@H]2C[C@@H](CCCNc3ccc4c(c3)C(=O)N(C3CCC(=O)NC3=O)C4=O)C2)cc1-c1cnc2ccc(C3CCOCC3)cc2n1] | 2-(2,6-dioxopiperidin-3-yl)-5-((3-(cis-3- (3-methyl-4-(7-(tetrahydro-2H-pyran-4- yl)quinoxalin-2-yl)-1H-pyrazol-1- yl)cyclobutyl)propyl)amino)isoindoline- 1,3-dione |
| GS-224 | 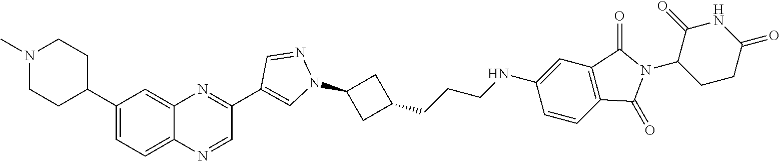 [SMILES: CN1CCC(c2ccc3ncc(-c4cnn([C@H]5C[C@H](CCCNc6ccc7c(c6)C(=O)N(C6CCC(=O)NC6=O)C7=O)C5)c4)nc3c2)CC1] | 2-(2,6-dioxopiperidin-3-yl)-5-((3-(trans- 3-(4-(7-(1-methylpiperidin-4- yl)quinoxalin-2-yl)-1H-pyrazol-1- yl)cyclobutyl)propyl)amino)isoindoline- 1,3-dione |
| GS-225 | 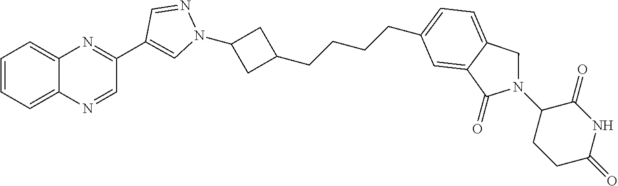 [SMILES: O=C1CCC(N2Cc3ccc(CCCCC4CC(n5cc(-c6cnc7ccccc7n6)cn5)C4)cc3C2=O)C(=O)N1] | 3-(1-oxo-6-(4-(3-(4-(quinoxalin-2-yl)- 1H-pyrazol-1- yl)cyclobutyl)butyl)isoindolin-2- yl)piperidine-2,6-dione |
| GS-226 | 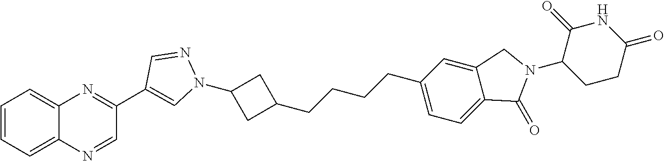 [SMILES: O=C1CCC(N2Cc3cc(CCCCC4CC(n5cc(-c6cnc7ccccc7n6)cn5)C4)ccc3C2=O)C(=O)N1] | 3-(1-oxo-5-(4-(3-(4-(quinoxalin-2-yl)- 1H-pyrazol-1- yl)cyclobutyl)butyl)isoindolin-2- yl)piperidine-2,6-dione |
| GS-227 | 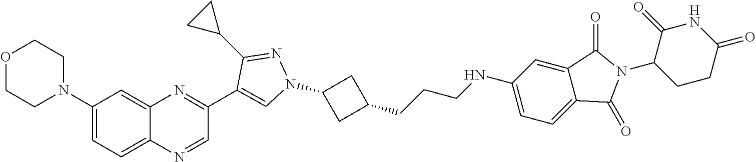 [SMILES: O=C1CCC(N2C(=O)c3ccc(NCCC[C@H]4C[C@@H](n5cc(-c6cnc7ccc(N8CCOCC8)cc7n6)c(C6CC6)n5)C4)cc3C2=O)C(=O)N1] | 5-((3-(cis-3-(3-cyclopropyl-4-(7- morpholinoquinoxalin-2-yl)-1H-pyrazol- 1-yl)cyclobutyl)propyl)amino)-2-(2,6- dioxopiperidin-3-yl)isoindoline-1,3-dione |
| GS-228 | 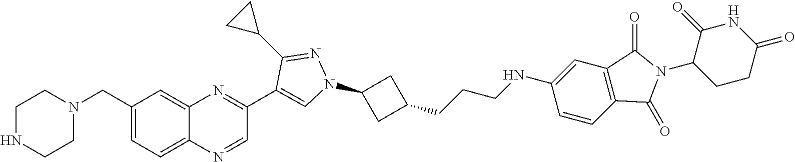 [SMILES: O=C1CCC(N2C(=O)c3ccc(NCCC[C@H]4C[C@H](n5cc(-c6cnc7ccc(CN8CCNCC8)cc7n6)c(C6CC6)n5)C4)cc3C2=O)C(=O)N1] | 5-((3-(trans-3-(3-cyclopropyl-4-(7- (piperazin-1-ylmethyl)quinoxalin-2-yl)- 1H-pyrazol-1- yl)cyclobutyl)propyl)amino)-2-(2,6- dioxopiperidin-3-yl)isoindoline-1,3-dione |
| GS-229 | 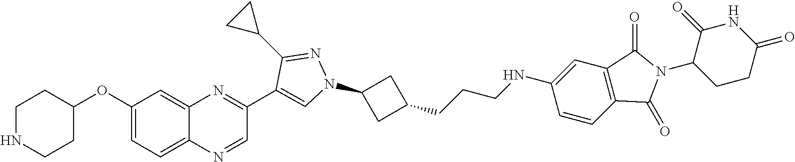 [SMILES: O=C1CCC(N2C(=O)c3ccc(NCCC[C@H]4C[C@H](n5cc(-c6cnc7ccc(OC8CCNCC8)cc7n6)c(C6CC6)n5)C4)cc3C2=O)C(=O)N1] | 5-((3-(trans-3-(3-cyclopropyl-4-(7- (piperidin-4-yloxy)quinoxalin-2-yl)-1H- pyrazol-1-yl)cyclobutyl)propyl)amino)-2- (2,6-dioxopiperidin-3-yl)isoindoline-1,3- dione |
| GS-230 | 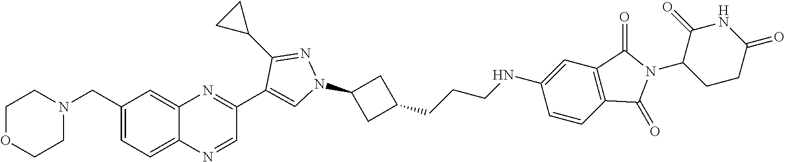 [SMILES: O=C1CCC(N2C(=O)c3ccc(NCCC[C@H]4C[C@H](n5cc(-c6cnc7ccc(CN8CCOCC8)cc7n6)c(C6CC6)n5)C4)cc3C2=O)C(=O)N1] | 5-((3-(trans-3-(3-cyclopropyl-4-(7- (morpholinomethyl)quinoxalin-2-yl)-1H- pyrazol-1-yl)cyclobutyl)propyl)amino)-2- (2,6-dioxopiperidin-3-yl)isoindoline-1,3- dione |
| GS-231 | 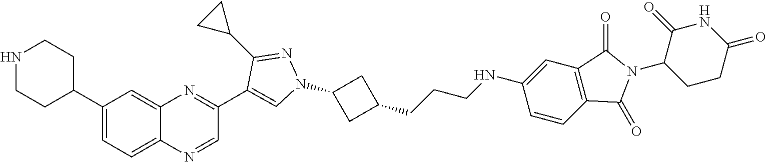 [SMILES: O=C1CCC(N2C(=O)c3ccc(NCCC[C@H]4C[C@@H](n5cc(-c6cnc7ccc(C8CCNCC8)cc7n6)c(C6CC6)n5)C4)cc3C2=O)C(=O)N1] | 5-((3-(cis-3-(3-cyclopropyl-4-(7- (piperidin-4-yl)quinoxalin-2-yl)-1H- pyrazol-1-yl)cyclobutyl)propyl)amino)-2- (2,6-dioxopiperidin-3-yl)isoindoline-1,3- dione |
| GS-232 | 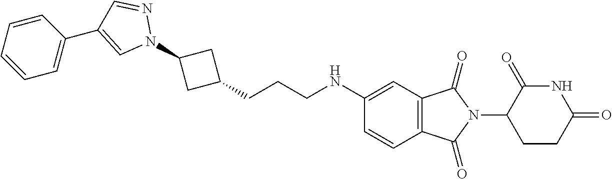 [SMILES: O=C1CCC(N2C(=O)c3ccc(NCCC[C@H]4C[C@H](n5cc(-c6ccccc6)cn5)C4)cc3C2=O)C(=O)N1] | 2-(2,6-dioxopiperidin-3-yl)-5-((3-(trans- 3-(4-phenyl-1H-pyrazol-1- yl)cyclobutyl)propyl)amino)isoindoline- 1,3-dione |
| GS-233 | 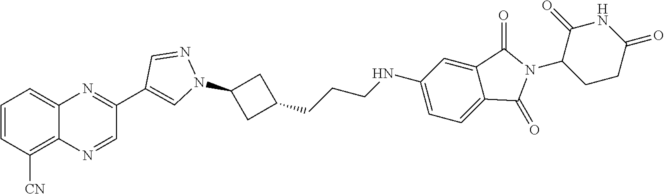 [SMILES: N#Cc1cccc2nc(-c3cnn([C@H]4C[C@H](CCCNc5ccc6c(c5)C(=O)N(C5CCC(=O)NC5=O)C6=O)C4)c3)cnc12] | 2-(1-(trans-3-(3-((2-(2,6-dioxopiperidin- 3-yl)-1,3-dioxoisoindolin-5- yl)amino)propyl)cyclobutyl)-1H-pyrazol- 4-yl)quinoxaline-5-carbonitrile |
| GS-234 | 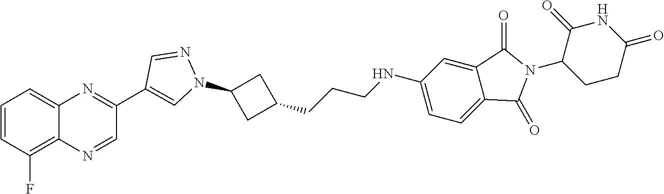 [SMILES: O=C1CCC(N2C(=O)c3ccc(NCCC[C@H]4C[C@H](n5cc(-c6cnc7c(F)cccc7n6)cn5)C4)cc3C2=O)C(=O)N1] | 2-(2,6-dioxopiperidin-3-yl)-5-((3-(trans- 3-(4-(5-fluoroquinoxalin-2-yl)-1H- pyrazol-1- yl)cyclobutyl)propyl)amino)isoindoline- 1,3-dione |
| GS-235 | 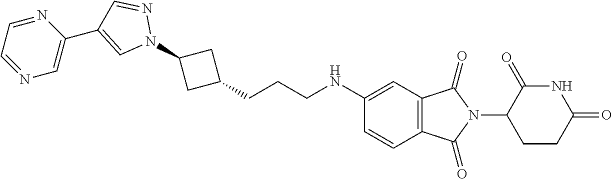 [SMILES: O=C1CCC(N2C(=O)c3ccc(NCCC[C@H]4C[C@H](n5cc(-c6cnccn6)cn5)C4)cc3C2=O)C(=O)N1] | 2-(2,6-dioxopiperidin-3-yl)-5-((3-(trans- 3-(4-(pyrazin-2-yl)-1H-pyrazol-1- yl)cyclobutyl)propyl)amino)isoindoline- 1,3-dione |
| GS-236 | 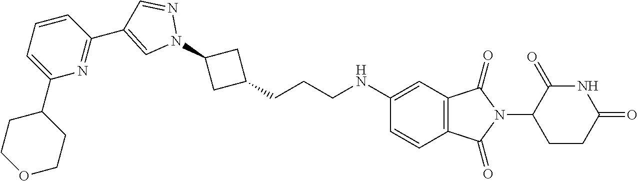 [SMILES: O=C1CCC(N2C(=O)c3ccc(NCCC[C@H]4C[C@H](n5cc(-c6cccc(C7CCOCC7)n6)cn5)C4)cc3C2=O)C(=O)N1] | 2-(2,6-dioxopiperidin-3-yl)-5-((3-(trans- 3-(4-(6-(tetrahydro-2H-pyran-4- yl)pyridin-2-yl)-1H-pyrazol-1- yl)cyclobutyl)propyl)amino)isoindoline- 1,3-dione |
| GS-237 | 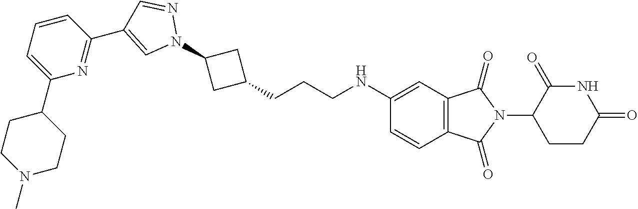 [SMILES: CN1CCC(c2cccc(-c3cnn([C@H]4C[C@H](CCCNc5ccc6c(c5)C(=O)N(C5CCC(=O)NC5=O)C6=O)C4)c3)n2)CC1] | 2-(2,6-dioxopiperidin-3-yl)-5-((3-(trans- 3-(4-(6-(1-methylpiperidin-4-yl)pyridin- 2-yl)-1H-pyrazol-1- yl)cyclobutyl)propyl)amino)isoindoline- 1,3-dione |
| GS-238 | 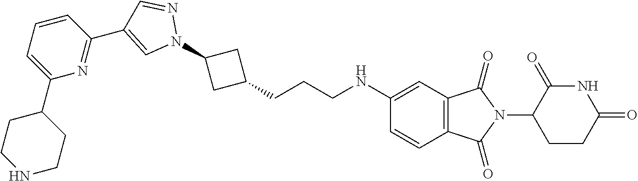 [SMILES: O=C1CCC(N2C(=O)c3ccc(NCCC[C@H]4C[C@H](n5cc(-c6cccc(C7CCNCC7)n6)cn5)C4)cc3C2=O)C(=O)N1] | 2-(2,6-dioxopiperidin-3-yl)-5-((3-(trans- 3-(4-(6-(piperidin-4-yl)pyridin-2-yl)-1H- pyrazol-1- yl)cyclobutyl)propyl)amino)isoindoline- 1,3-dione |
| GS-239 | 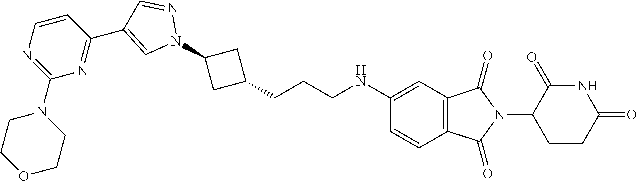 [SMILES: O=C1CCC(N2C(=O)c3ccc(NCCC[C@H]4C[C@H](n5cc(-c6ccnc(N7CCOCC7)n6)cn5)C4)cc3C2=O)C(=O)N1] | 2-(2,6-dioxopiperidin-3-yl)-5-((3-(trans- 3-(4-(2-morpholinopyrimidin-4-yl)-1H- pyrazol-1- yl)cyclobutyl)propyl)amino)isoindoline- 1,3-dione |
| GS-240 | 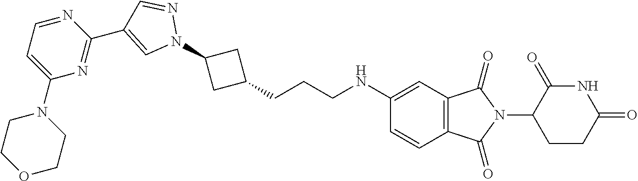 [SMILES: O=C1CCC(N2C(=O)c3ccc(NCCC[C@H]4C[C@H](n5cc(-c6nccc(N7CCOCC7)n6)cn5)C4)cc3C2=O)C(=O)N1] | 2-(2,6-dioxopiperidin-3-yl)-5-((3-(trans- 3-(4-(4-morpholinopyrimidin-2-yl)-1H- pyrazol-1- yl)cyclobutyl)propyl)amino)isoindoline- 1,3-dione |
| GS-241 | 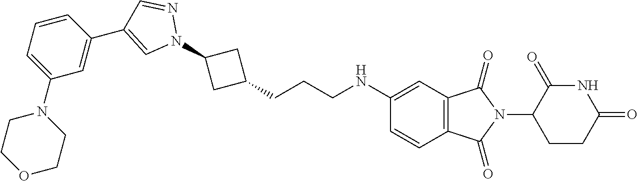 [SMILES: O=C1CCC(N2C(=O)c3ccc(NCCC[C@H]4C[C@H](n5cc(-c6cccc(N7CCOCC7)c6)cn5)C4)cc3C2=O)C(=O)N1] | 2-(2,6-dioxopiperidin-3-yl)-5-((3-(trans- 3-(4-(3-morpholinophenyl)-1H-pyrazol- 1- yl)cyclobutyl)propyl)amino)isoindoline- 1,3-dione |
| GS-242 | 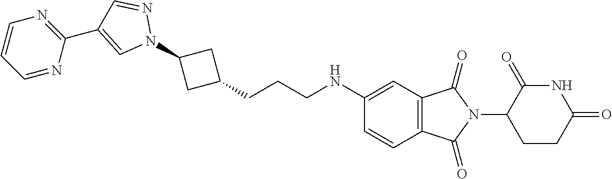 [SMILES: O=C1CCC(N2C(=O)c3ccc(NCCC[C@H]4C[C@H](n5cc(-c6ncccn6)cn5)C4)cc3C2=O)C(=O)N1] | 2-(2,6-dioxopiperidin-3-yl)-5-((3-(trans- 3-(4-(pyrimidin-2-yl)-1H-pyrazol-1- yl)cyclobutyl)propyl)amino)isoindoline- 1,3-dione |
| GS-243 | 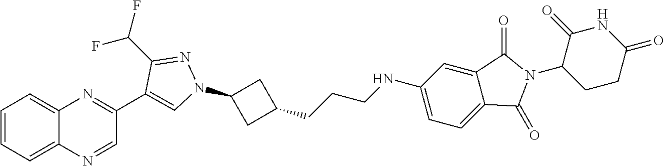 [SMILES: O=C1CCC(N2C(=O)c3ccc(NCCC[C@H]4C[C@H](n5cc(-c6cnc7ccccc7n6)c(C(F)F)n5)C4)cc3C2=O)C(=O)N1] | 5-((3-(trans-3-(3-(difluoromethyl)-4- (quinoxalin-2-yl)-1H-pyrazol-1- yl)cyclobutyl)propyl)amino)-2-(2,6- dioxopiperidin-3-yl)isoindoline-1,3-dione |
| GS-244 | 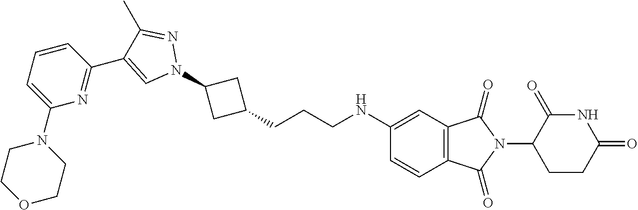 [SMILES: Cc1nn([C@H]2C[C@H](CCCNc3ccc4c(c3)C(=O)N(C3CCC(=O)NC3=O)C4=O)C2)cc1-c1cccc(N2CCOCC2)n1] | 2-(2,6-dioxopiperidin-3-yl)-5-((3-(trans- 3-(3-methyl-4-(6-morpholinopyridin-2- yl)-1H-pyrazol-1- yl)cyclobutyl)propyl)amino)isoindoline- 1,3-dione |
| GS-245 | 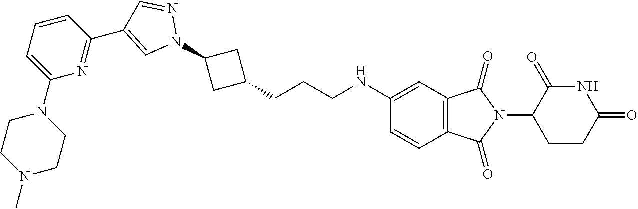 [SMILES: CN1CCN(c2cccc(-c3cnn([C@H]4C[C@H](CCCNc5ccc6c(c5)C(=O)N(C5CCC(=O)NC5=O)C6=O)C4)c3)n2)CC1] | 2-(2,6-dioxopiperidin-3-yl)-5-((3-(trans- 3-(4-(6-(4-methylpiperazin-1-yl)pyridin- 2-yl)-1H-pyrazol-1- yl)cyclobutyl)propyl)amino)isoindoline- 1,3-dione |
| GS-246 | 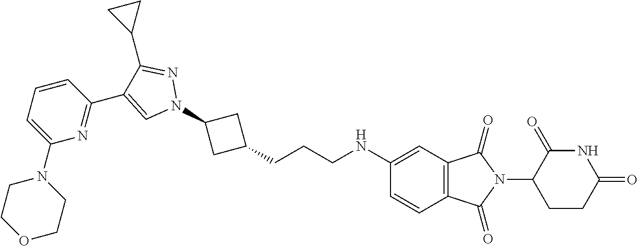 [SMILES: O=C1CCC(N2C(=O)c3ccc(NCCC[C@H]4C[C@H](n5cc(-c6cccc(N7CCOCC7)n6)c(C6CC6)n5)C4)cc3C2=O)C(=O)N1] | 5-((3-(trans-3-(3-cyclopropyl-4-(6- morpholinopyridin-2-yl)-1H-pyrazol-1- yl)cyclobutyl)propyl)amino)-2-(2,6- dioxopiperidin-3-yl)isoindoline-1,3-dione |
| GS-247 | 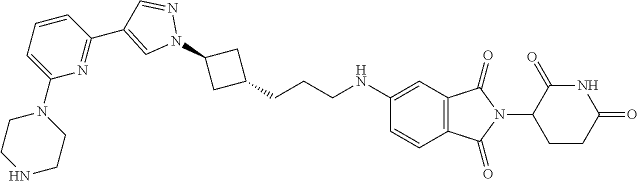 [SMILES: O=C1CCC(N2C(=O)c3ccc(NCCC[C@H]4C[C@H](n5cc(-c6cccc(N7CCNCC7)n6)cn5)C4)cc3C2=O)C(=O)N1] | 2-(2,6-dioxopiperidin-3-yl)-5-((3-(trans- 3-(4-(6-(piperazin-1-yl)pyridin-2-yl)-1H- pyrazol-1- yl)cyclobutyl)propyl)amino)isoindoline- 1,3-dione |
| GS-248 | 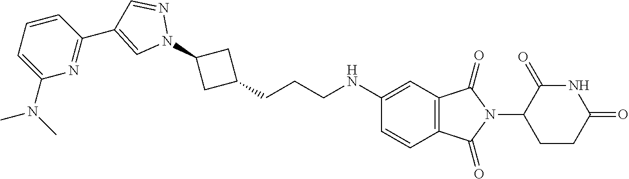 [SMILES: CN(C)c1cccc(-c2cnn([C@H]3C[C@H](CCCNc4ccc5c(c4)C(=O)N(C4CCC(=O)NC4=O)C5=O)C3)c2)n1] | 5-((3-(trans-3-(4-(6- (dimethylamino)pyridin-2-yl)-1H- pyrazol-1-yl)cyclobutyl)propyl)amino)-2- (2,6-dioxopiperidin-3-yl)isoindoline-1,3- dione |
| GS-249 | 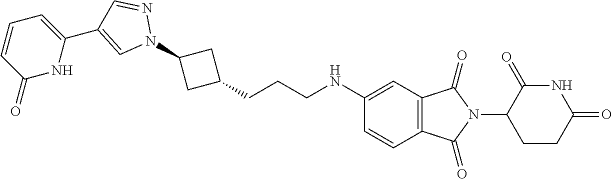 [SMILES: O=C1CCC(N2C(=O)c3ccc(NCCC[C@H]4C[C@H](n5cc(-c6cccc(=O)[nH]6)cn5)C4)cc3C2=O)C(=O)N1] | 2-(2,6-dioxopiperidin-3-yl)-5-((3-(trans- 3-(4-(6-oxo-1,6-dihydropyridin-2-yl)-1H- pyrazol-1- yl)cyclobutyl)propyl)amino)isoindoline- 1,3-dione |
| GS-250 | 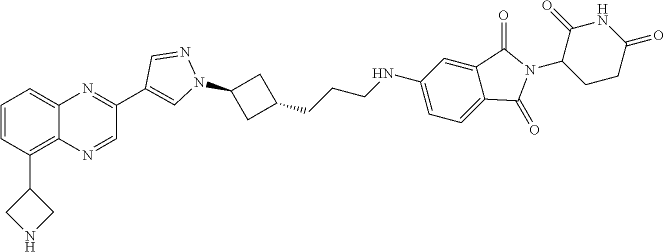 [SMILES: O=C1CCC(N2C(=O)c3ccc(NCCC[C@H]4C[C@H](n5cc(-c6cnc7c(C8CNC8)cccc7n6)cn5)C4)cc3C2=O)C(=O)N1] | 5-((3-(trans-3-(4-(5-(azetidin-3- yl)quinoxalin-2-yl)-1H-pyrazol-1- yl)cyclobutyl)propyl)amino)-2-(2,6- dioxopiperidin-3-yl)isoindoline-1,3-dione |
| GS-251 | 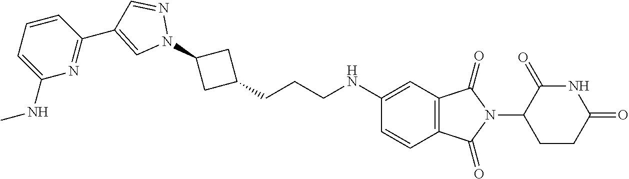 [SMILES: CNc1cccc(-c2cnn([C@H]3C[C@H](CCCNc4ccc5c(c4)C(=O)N(C4CCC(=O)NC4=O)C5=O)C3)c2)n1] | 2-(2,6-dioxopiperidin-3-yl)-5-((3-(trans- 3-(4-(6-(methylamino)pyridin-2-yl)-1H- pyrazol-1- yl)cyclobutyl)propyl)amino)isoindoline- 1,3-dione |
| GS-252 | 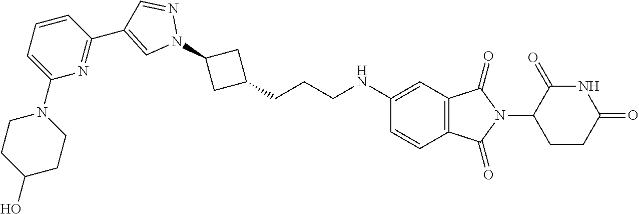 [SMILES: O=C1CCC(N2C(=O)c3ccc(NCCC[C@H]4C[C@H](n5cc(-c6cccc(N7CCC(O)CC7)n6)cn5)C4)cc3C2=O)C(=O)N1] | 2-(2,6-dioxopiperidin-3-yl)-5-((3-(trans- 3-(4-(6-(4-hydroxypiperidin-1-yl)pyridin- 2-yl)-1H-pyrazol-1- yl)cyclobutyl)propyl)amino)isoindoline- 1,3-dione |
| GS-253 | 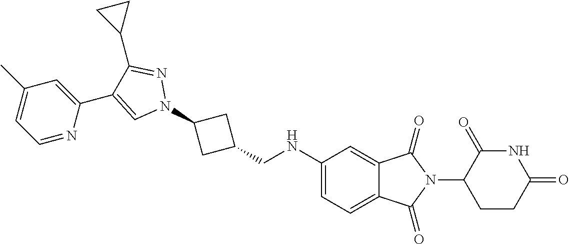 [SMILES: Cc1ccnc(-c2cn([C@H]3C[C@H](CNc4ccc5c(c4)C(=O)N(C4CCC(=O)NC4=O)C5=O)C3)nc2C2CC2)c1] | 5-(((trans-3-(3-cyclopropyl-4-(4- methylpyridin-2-yl)-1H-pyrazol-1- yl)cyclobutyl)methyl)amino)-2-(2,6- dioxopiperidin-3-yl)isoindoline-1,3-dione |
| GS-254 | 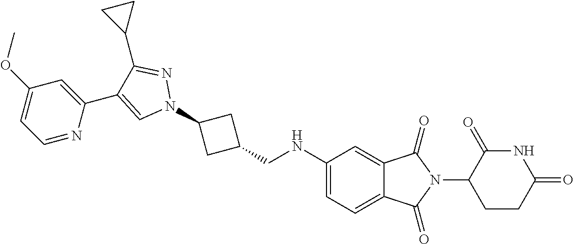 [SMILES: COc1ccnc(-c2cn([C@H]3C[C@H](CNc4ccc5c(c4)C(=O)N(C4CCC(=O)NC4=O)C5=O)C3)nc2C2CC2)c1] | 5-(((trans-3-(3-cyclopropyl-4-(4- methoxypyridin-2-yl)-1H-pyrazol-1- yl)cyclobutyl)methyl)amino)-2-(2,6- dioxopiperidin-3-yl)isoindoline-1,3-dione |
| GS-255 | 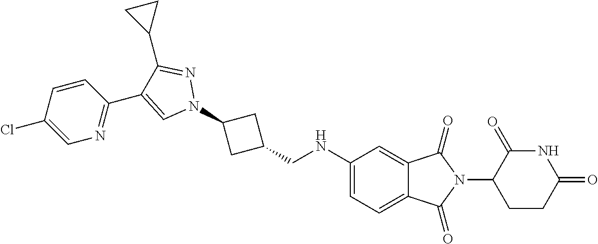 [SMILES: O=C1CCC(N2C(=O)c3ccc(NC[C@H]4C[C@H](n5cc(-c6ccc(Cl)cn6)c(C6CC6)n5)C4)cc3C2=O)C(=O)N1] | 5-(((trans-3-(4-(5-chloropyridin-2-yl)-3- cyclopropyl-1H-pyrazol-1- yl)cyclobutyl)methyl)amino)-2-(2,6- dioxopiperidin-3-yl)isoindoline-1,3-dione |
| GS-256 | 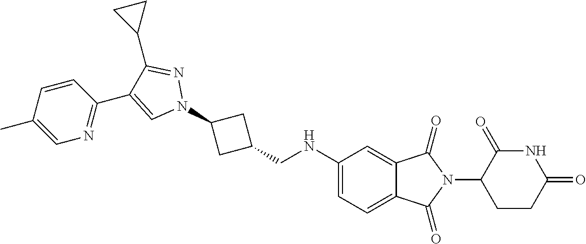 [SMILES: Cc1ccc(-c2cn([C@H]3C[C@H](CNc4ccc5c(c4)C(=O)N(C4CCC(=O)NC4=O)C5=O)C3)nc2C2CC2)nc1] | 5-(((trans-3-(3-cyclopropyl-4-(5- methylpyridin-2-yl)-1H-pyrazol-1- yl)cyclobutyl)methyl)amino)-2-(2,6- dioxopiperidin-3-yl)isoindoline-1,3-dione |
| GS-257 | 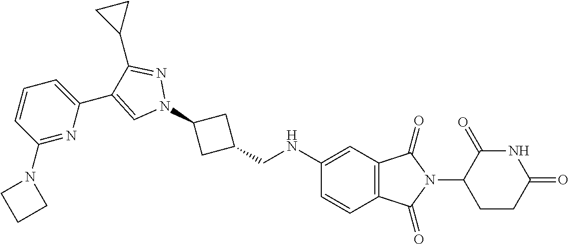 [SMILES: O=C1CCC(N2C(=O)c3ccc(NC[C@H]4C[C@H](n5cc(-c6cccc(N7CCC7)n6)c(C6CC6)n5)C4)cc3C2=O)C(=O)N1] | 5-(((trans-3-(4-(6-(azetidin-1-yl)pyridin- 2-yl)-3-cyclopropyl-1H-pyrazol-1- yl)cyclobutyl)methyl)amino)-2-(2,6- dioxopiperidin-3-yl)isoindoline-1,3-dione |
| GS-258 | 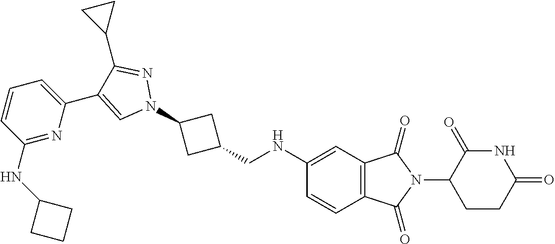 [SMILES: O=C1CCC(N2C(=O)c3ccc(NC[C@H]4C[C@H](n5cc(-c6cccc(NC7CCC7)n6)c(C6CC6)n5)C4)cc3C2=O)C(=O)N1] | 5-(((trans-3-(4-(6- (cyclobutylamino)pyridin-2-yl)-3- cyclopropyl-1H-pyrazol-1- yl)cyclobutyl)methyl)amino)-2-(2,6- dioxopiperidin-3-yl)isoindoline-1,3-dione |
| GS-259 | 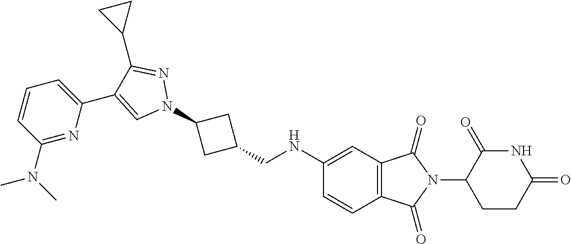 [SMILES: CN(C)c1cccc(-c2cn([C@H]3C[C@H](CNc4ccc5c(c4)C(=O)N(C4CCC(=O)NC4=O)C5=O)C3)nc2C2CC2)n1] | 5-(((trans-3-(3-cyclopropyl-4-(6- (dimethylamino)pyridin-2-yl)-1H- pyrazol-1-yl)cyclobutyl)methyl)amino)- 2-(2,6-dioxopiperidin-3-yl)isoindoline- 1,3-dione |
| GS-260 | 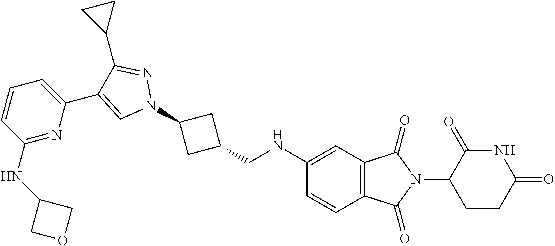 [SMILES: O=C1CCC(N2C(=O)c3ccc(NC[C@H]4C[C@H](n5cc(-c6cccc(NC7COC7)n6)c(C6CC6)n5)C4)cc3C2=O)C(=O)N1] | 5-(((trans-3-(3-cyclopropyl-4-(6-(oxetan- 3-ylamino)pyridin-2-yl)-1H-pyrazol-1- yl)cyclobutyl)methyl)amino)-2-(2,6- dioxopiperidin-3-yl)isoindoline-1,3-dione |
| GS-261 | 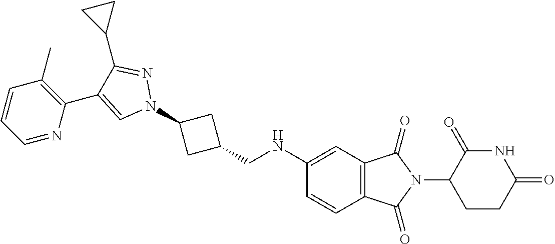 [SMILES: Cc1cccnc1-c1cn([C@H]2C[C@H](CNc3ccc4c(c3)C(=O)N(C3CCC(=O)NC3=O)C4=O)C2)nc1C1CC1] | 5-(((trans-3-(3-cyclopropyl-4-(3- methylpyridin-2-yl)-1H-pyrazol-1- yl)cyclobutyl)methyl)amino)-2-(2,6- dioxopiperidin-3-yl)isoindoline-1,3-dione |
| GS-262 | 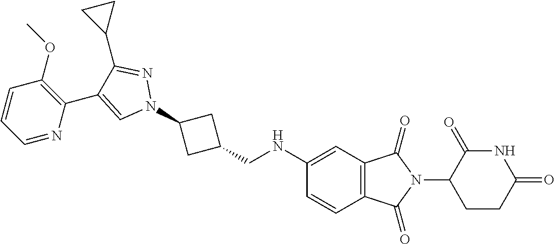 [SMILES: COc1cccnc1-c1cn([C@H]2C[C@H](CNc3ccc4c(c3)C(=O)N(C3CCC(=O)NC3=O)C4=O)C2)nc1C1CC1] | 5-(((trans-3-(3-cyclopropyl-4-(3- methoxypyridin-2-yl)-1H-pyrazol-1- yl)cyclobutyl)methyl)amino)-2-(2,6- dioxopiperidin-3-yl)isoindoline-1,3-dione |
| GS-263 | 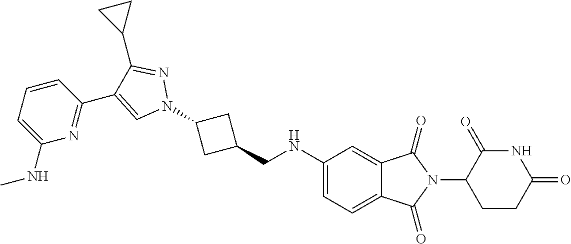 [SMILES: CNc1cccc(-c2cn([C@H]3C[C@H](CNc4ccc5c(c4)C(=O)N(C4CCC(=O)NC4=O)C5=O)C3)nc2C2CC2)n1] | 5-(((trans-3-(3-cyclopropyl-4-(6- (methylamino)pyridin-2-yl)-1H-pyrazol- 1-yl)cyclobutyl)methyl)amino)-2-(2,6- dioxopiperidin-3-yl)isoindoline-1,3-dione |
| GS-264 | 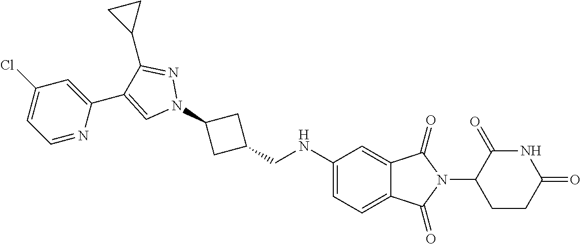 [SMILES: O=C1CCC(N2C(=O)c3ccc(NC[C@H]4C[C@H](n5cc(-c6cc(Cl)ccn6)c(C6CC6)n5)C4)cc3C2=O)C(=O)N1] | 5-(((trans-3-(4-(4-chloropyridin-2-yl)-3- cyclopropyl-1H-pyrazol-1- yl)cyclobutyl)methyl)amino)-2-(2,6- dioxopiperidin-3-yl)isoindoline-1,3-dione |
| GS-265 | 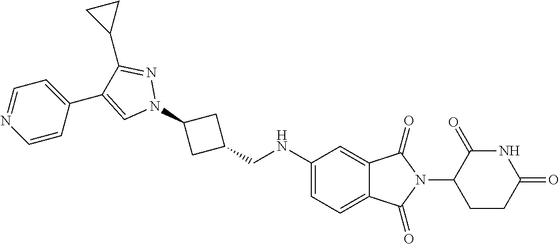 [SMILES: O=C1CCC(N2C(=O)c3ccc(NC[C@H]4C[C@H](n5cc(-c6ccncc6)c(C6CC6)n5)C4)cc3C2=O)C(=O)N1] | 5-(((trans-3-(3-cyclopropyl-4-(pyridin-4- yl)-1H-pyrazol-1- yl)cyclobutyl)methyl)amino)-2-(2,6- dioxopiperidin-3-yl)isoindoline-1,3-dione |
| GS-266 | 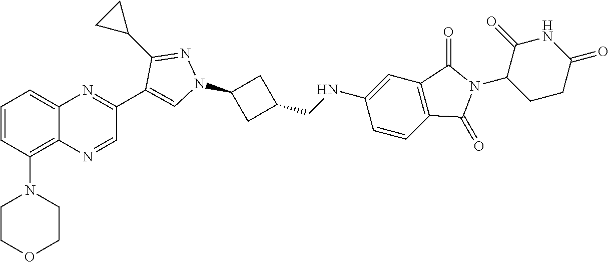 [SMILES: O=C1CCC(N2C(=O)c3ccc(NC[C@H]4C[C@H](n5cc(-c6cnc7c(N8CCOCC8)cccc7n6)c(C6CC6)n5)C4)cc3C2=O)C(=O)N1] | 5-(((trans-3-(3-cyclopropyl-4-(5- morpholinoquinoxalin-2-yl)-1H-pyrazol- 1-yl)cyclobutyl)methyl)amino)-2-(2,6- dioxopiperidin-3-yl)isoindoline-1,3-dione |
| GS-267 | 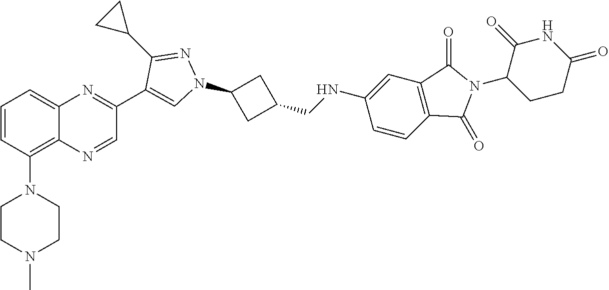 [SMILES: CN1CCN(c2cccc3nc(-c4cn([C@H]5C[C@H](CNc6ccc7c(c6)C(=O)N(C6CCC(=O)NC6=O)C7=O)C5)nc4C4CC4)cnc23)CC1] | 5-(((trans-3-(3-cyclopropyl-4-(5-(4- methylpiperazin-1-yl)quinoxalin-2-yl)- 1H-pyrazol-1- yl)cyclobutyl)methyl)amino)-2-(2,6- dioxopiperidin-3-yl)isoindoline-1,3-dione |
| GS-268 (CG6328) | 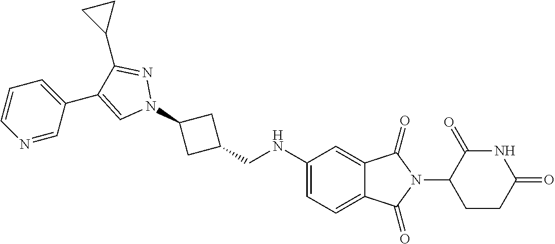 [SMILES: O=C1CCC(N2C(=O)c3ccc(NC[C@H]4C[C@H](n5cc(-c6cccnc6)c(C6CC6)n5)C4)cc3C2=O)C(=O)N1] | 5-(((trans-3-(3-cyclopropyl-4-(pyridin-3- yl)-1H-pyrazol-1- yl)cyclobutyl)methyl)amino)-2-(2,6- dioxopiperidin-3-yl)isoindoline-1,3-dione |
| GS-269 | 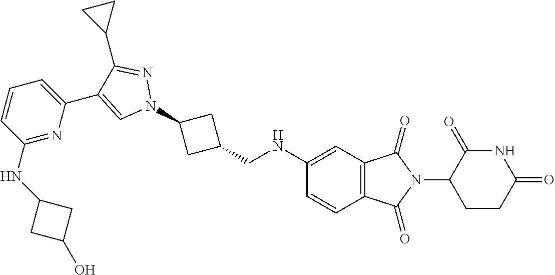 [SMILES: O=C1CCC(N2C(=O)c3ccc(NC[C@H]4C[C@H](n5cc(-c6cccc(NC7CC(O)C7)n6)c(C6CC6)n5)C4)cc3C2=O)C(=O)N1] | 5-(((trans-3-(3-cyclopropyl-4-(6-((3- hydroxycyclobutyl)amino)pyridin-2-yl)- 1H-pyrazol-1- yl)cyclobutyl)methyl)amino)-2-(2,6- dioxopiperidin-3-yl)isoindoline-1,3-dione |
| GS-270 | 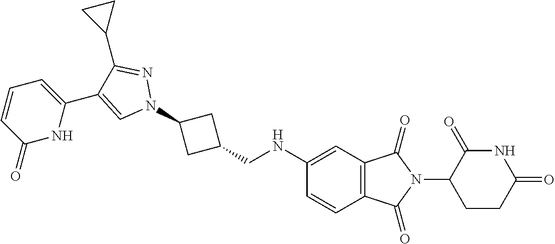 [SMILES: O=C1CCC(N2C(=O)c3ccc(NC[C@H]4C[C@H](n5cc(-c6cccc(=O)[nH]6)c(C6CC6)n5)C4)cc3C2=O)C(=O)N1] | 5-(((trans-3-(3-cyclopropyl-4-(6-oxo-1,6- dihydropyridin-2-yl)-1H-pyrazol-1- yl)cyclobutyl)methyl)amino)-2-(2,6- dioxopiperidin-3-yl)isoindoline-1,3-dione |
| GS-271 | 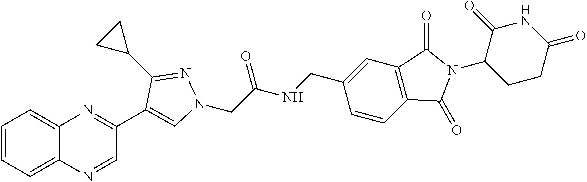 [SMILES: O=C(Cn1cc(-c2cnc3ccccc3n2)c(C2CC2)n1)NCc1ccc2c(c1)C(=O)N(C1CCC(=O)NC1=O)C2=O] | 2-(3-cyclopropyl-4-(quinoxalin-2-yl)-1H- pyrazol-1-yl)-N-((2-(2,6-dioxopiperidin- 3-yl)-1,3-dioxoisoindolin-5- yl)methyl)acetamide |
| GS-272 | 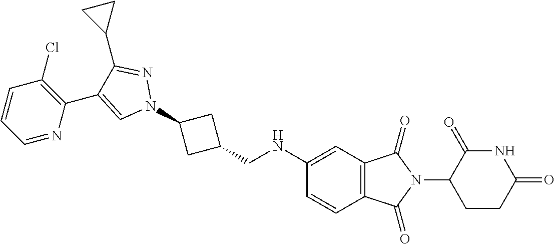 [SMILES: O=C1CCC(N2C(=O)c3ccc(NC[C@H]4C[C@H](n5cc(-c6ncccc6Cl)c(C6CC6)n5)C4)cc3C2=O)C(=O)N1] | 5-(((trans-3-(4-(3-chloropyridin-2-yl)-3- cyclopropyl-1H-pyrazol-1- yl)cyclobutyl)methyl)amino)-2-(2,6- dioxopiperidin-3-yl)isoindoline-1,3-dione |
| GS-273 | 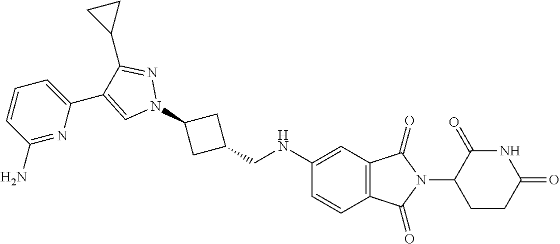 [SMILES: Nc1cccc(-c2cn([C@H]3C[C@H](CNc4ccc5c(c4)C(=O)N(C4CCC(=O)NC4=O)C5=O)C3)nc2C2CC2)n1] | 5-(((trans-3-(4-(6-aminopyridin-2-yl)-3- cyclopropyl-1H-pyrazol-1- yl)cyclobutyl)methyl)amino)-2-(2,6- dioxopiperidin-3-yl)isoindoline-1,3-dione |
| GS-274 | 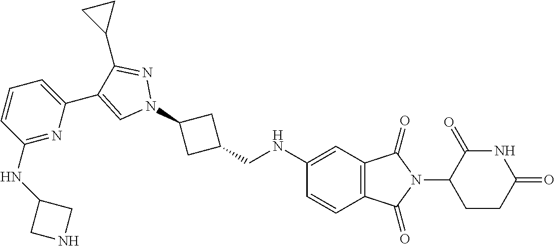 [SMILES: O=C1CCC(N2C(=O)c3ccc(NC[C@H]4C[C@H](n5cc(-c6cccc(NC7CNC7)n6)c(C6CC6)n5)C4)cc3C2=O)C(=O)N1] | 5-(((trans-3-(4-(6-(azetidin-3- ylamino)pyridin-2-yl)-3-cyclopropyl-1H- pyrazol-1-yl)cyclobutyl)methyl)amino)- 2-(2,6-dioxopiperidin-3-yl)isoindoline- 1,3-dione |
| GS-275 | 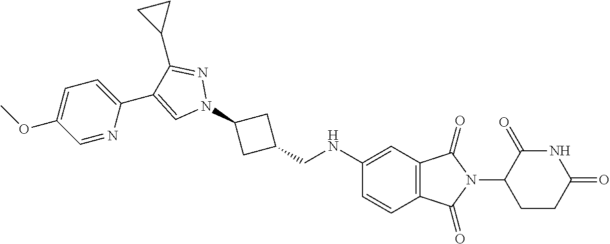 [SMILES: COc1ccc(-c2cn([C@H]3C[C@H](CNc4ccc5c(c4)C(=O)N(C4CCC(=O)NC4=O)C5=O)C3)nc2C2CC2)nc1] | 5-(((trans-3-(3-cyclopropyl-4-(5- methoxypyridin-2-yl)-1H-pyrazol-1- yl)cyclobutyl)methyl)amino)-2-(2,6- dioxopiperidin-3-yl)isoindoline-1,3-dione |
| GS-276 | 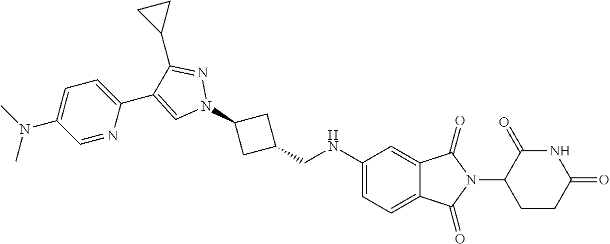 [SMILES: CN(C)c1ccc(-c2cn([C@H]3C[C@H](CNc4ccc5c(c4)C(=O)N(C4CCC(=O)NC4=O)C5=O)C3)nc2C2CC2)nc1] | 5-(((trans-3-(3-cyclopropyl-4-(5- (dimethylamino)pyridin-2-yl)-1H- pyrazol-1-yl)cyclobutyl)methyl)amino)- 2-(2,6-dioxopiperidin-3-yl)isoindoline- 1,3-dione |
| GS-277 | 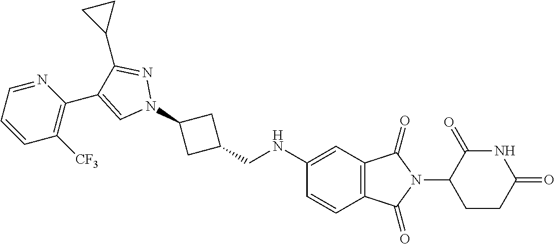 [SMILES: O=C1CCC(N2C(=O)c3ccc(NC[C@H]4C[C@H](n5cc(-c6ncccc6C(F)(F)F)c(C6CC6)n5)C4)cc3C2=O)C(=O)N1] | 5-(((trans-3-(3-cyclopropyl-4-(3- (trifluoromethyl)pyridin-2-yl)-1H- pyrazol-1-yl)cyclobutyl)methyl)amino)- 2-(2,6-dioxopiperidin-3-yl)isoindoline- 1,3-dione |
| GS-278 | 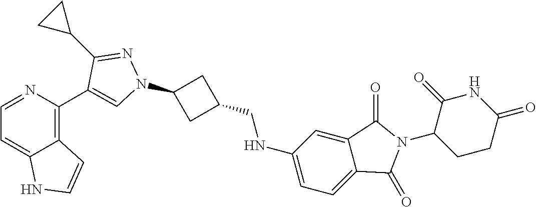 [SMILES: O=C1CCC(N2C(=O)c3ccc(NC[C@H]4C[C@H](n5cc(-c6nccc7[nH]ccc67)c(C6CC6)n5)C4)cc3C2=O)C(=O)N1] | 5-(((trans-3-(3-cyclopropyl-4-(1H- pyrrolo[3,2-c]pyridin-4-yl)-1H-pyrazol-1- yl)cyclobutyl)methyl)amino)-2-(2,6- dioxopiperidin-3-yl)isoindoline-1,3-dione |
| GS-279 | 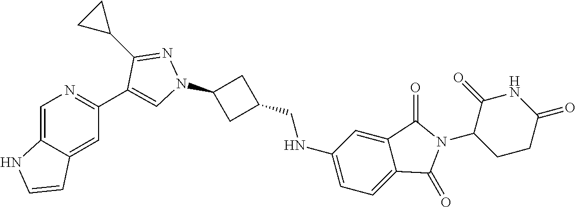 [SMILES: O=C1CCC(N2C(=O)c3ccc(NC[C@H]4C[C@H](n5cc(-c6cc7cc[nH]c7cn6)c(C6CC6)n5)C4)cc3C2=O)C(=O)N1] | 5-(((trans-3-(3-cyclopropyl-4-(1H- pyrrolo[2,3-c]pyridin-5-yl)-1H-pyrazol-1- yl)cyclobutyl)methyl)amino)-2-(2,6- dioxopiperidin-3-yl)isoindoline-1,3-dione |
| GS-280 | 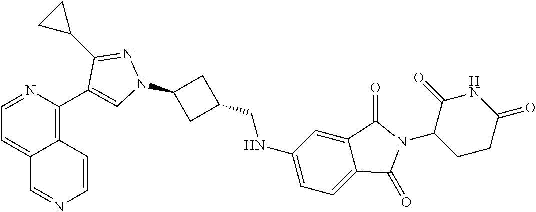 [SMILES: O=C1CCC(N2C(=O)c3ccc(NC[C@H]4C[C@H](n5cc(-c6nccc7cnccc67)c(C6CC6)n5)C4)cc3C2=O)C(=O)N1] | 5-(((trans-3-(3-cyclopropyl-4-(2,6- naphthyridin-1-yl)-1H-pyrazol-1- yl)cyclobutyl)methyl)amino)-2-(2,6- dioxopiperidin-3-yl)isoindoline-1,3-dione |
| GS-281 | 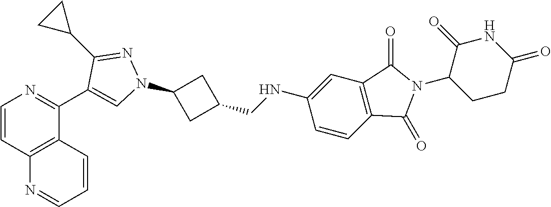 [SMILES: O=C1CCC(N2C(=O)c3ccc(NC[C@H]4C[C@H](n5cc(-c6nccc7ncccc67)c(C6CC6)n5)C4)cc3C2=O)C(=O)N1] | 5-(((trans-3-(3-cyclopropyl-4-(1,6- naphthyridin-5-yl)-1H-pyrazol-1- yl)cyclobutyl)methyl)amino)-2-(2,6- dioxopiperidin-3-yl)isoindoline-1,3-dione |
| GS-282 | 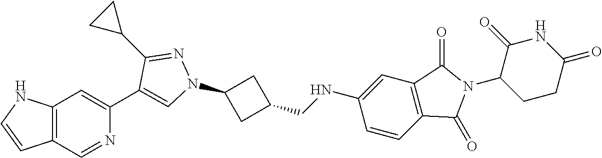 [SMILES: O=C1CCC(N2C(=O)c3ccc(NC[C@H]4C[C@H](n5cc(-c6cc7[nH]ccc7cn6)c(C6CC6)n5)C4)cc3C2=O)C(=O)N1] | 5-(((trans-3-(3-cyclopropyl-4-(1H- pyrrolo[3,2-c]pyridin-6-yl)-1H-pyrazol-1- yl)cyclobutyl)methyl)amino)-2-(2,6- dioxopiperidin-3-yl)isoindoline-1,3-dione |
| GS-283 | 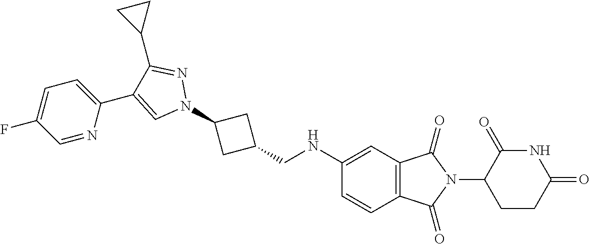 [SMILES: O=C1CCC(N2C(=O)c3ccc(NC[C@H]4C[C@H](n5cc(-c6ccc(F)cn6)c(C6CC6)n5)C4)cc3C2=O)C(=O)N1] | 5-(((trans-3-(3-cyclopropyl-4-(5- fluoropyridin-2-yl)-1H-pyrazol-1- yl)cyclobutyl)methyl)amino)-2-(2,6- dioxopiperidin-3-yl)isoindoline-1,3-dione |
| GS-284 | 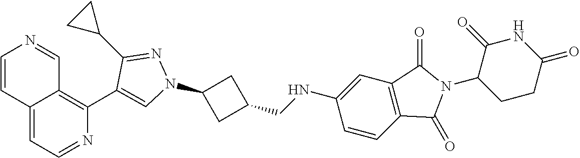 [SMILES: O=C1CCC(N2C(=O)c3ccc(NC[C@H]4C[C@H](n5cc(-c6nccc7ccncc67)c(C6CC6)n5)C4)cc3C2=O)C(=O)N1] | 5-(((trans-3-(3-cyclopropyl-4-(2,7- naphthyridin-1-yl)-1H-pyrazol-1- yl)cyclobutyl)methyl)amino)-2-(2,6- dioxopiperidin-3-yl)isoindoline-1,3-dione |
| GS-285 | 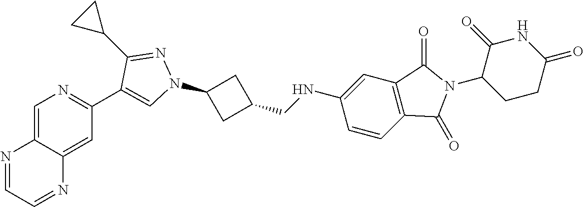 [SMILES: O=C1CCC(N2C(=O)c3ccc(NC[C@H]4C[C@H](n5cc(-c6cc7nccnc7cn6)c(C6CC6)n5)C4)cc3C2=O)C(=O)N1] | 5-(((trans-3-(3-cyclopropyl-4- (pyrido[3,4-b]pyrazin-7-yl)-1H-pyrazol- 1-yl)cyclobutyl)methyl)amino)-2-(2,6- dioxopiperidin-3-yl)isoindoline-1,3-dione |
| GS-286 | 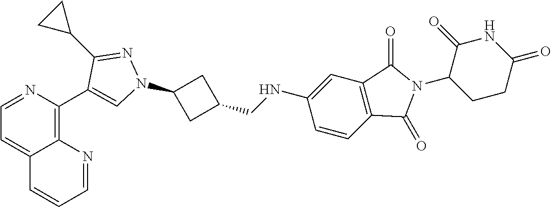 [SMILES: O=C1CCC(N2C(=O)c3ccc(NC[C@H]4C[C@H](n5cc(-c6nccc7cccnc67)c(C6CC6)n5)C4)cc3C2=O)C(=O)N1] | 5-(((trans-3-(3-cyclopropyl-4-(1,7- naphthyridin-8-yl)-1H-pyrazol-1- yl)cyclobutyl)methyl)amino)-2-(2,6- dioxopiperidin-3-yl)isoindoline-1,3-dione |
| GS-287 | 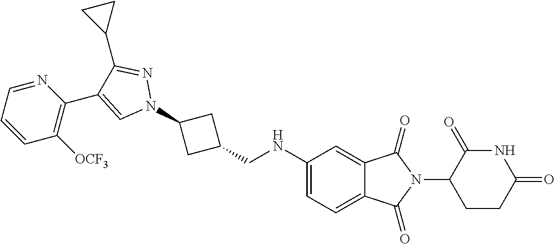 [SMILES: O=C1CCC(N2C(=O)c3ccc(NC[C@H]4C[C@H](n5cc(-c6ncccc6OC(F)(F)F)c(C6CC6)n5)C4)cc3C2=O)C(=O)N1] | 5-(((trans-3-(3-cyclopropyl-4-(3- (trifluoromethoxy)pyridin-2-yl)-1H- pyrazol-1-yl)cyclobutyl)methyl)amino)- 2-(2,6-dioxopiperidin-3-yl)isoindoline- 1,3-dione |
| GS-288 | 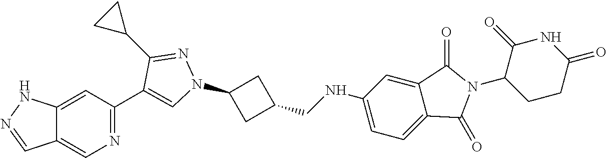 [SMILES: O=C1CCC(N2C(=O)c3ccc(NC[C@H]4C[C@H](n5cc(-c6cc7[nH]ncc7cn6)c(C6CC6)n5)C4)cc3C2=O)C(=O)N1] | 5-(((trans-3-(3-cyclopropyl-4-(1H- pyrazolo[4,3-c]pyridin-6-yl)-1H-pyrazol- 1-yl)cyclobutyl)methyl)amino)-2-(2,6- dioxopiperidin-3-yl)isoindoline-1,3-dione |
| GS-289 | 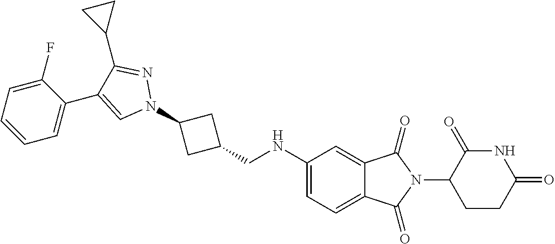 [SMILES: O=C1CCC(N2C(=O)c3ccc(NC[C@H]4C[C@H](n5cc(-c6ccccc6F)c(C6CC6)n5)C4)cc3C2=O)C(=O)N1] | 5-(((trans-3-(3-cyclopropyl-4-(2- fluorophenyl)-1H-pyrazol-1- yl)cyclobutyl)methyl)amino)-2-(2,6- dioxopiperidin-3-yl)isoindoline-1,3-dione |
| GS-290 | 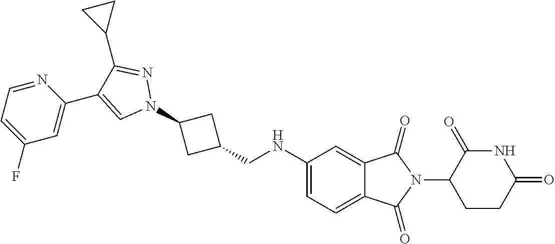 [SMILES: O=C1CCC(N2C(=O)c3ccc(NC[C@H]4C[C@H](n5cc(-c6cc(F)ccn6)c(C6CC6)n5)C4)cc3C2=O)C(=O)N1] | 5-(((trans-3-(3-cyclopropyl-4-(4- fluoropyridin-2-yl)-1H-pyrazol-1- yl)cyclobutyl)methyl)amino)-2-(2,6- dioxopiperidin-3-yl)isoindoline-1,3-dione |
| GS-291 | 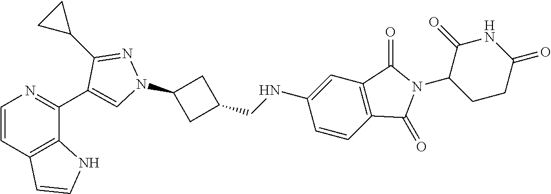 [SMILES: O=C1CCC(N2C(=O)c3ccc(NC[C@H]4C[C@H](n5cc(-c6nccc7cc[nH]c67)c(C6CC6)n5)C4)cc3C2=O)C(=O)N1] | 5-(((trans-3-(3-cyclopropyl-4-(1H- pyrrolo[2,3-c]pyridin-7-yl)-1H-pyrazol-1- yl)cyclobutyl)methyl)amino)-2-(2,6- dioxopiperidin-3-yl)isoindoline-1,3-dione |
| GS-292 | 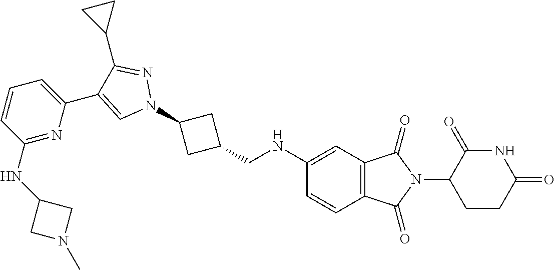 [SMILES: CN1CC(Nc2cccc(-c3cn([C@H]4C[C@H](CNc5ccc6c(c5)C(=O)N(C5CCC(=O)NC5=O)C6=O)C4)nc3C3CC3)n2)C1] | 5-(((trans-3-(3-cyclopropyl-4-(6-((1- methylazetidin-3-yl)amino)pyridin-2-yl)- 1H-pyrazol-1- yl)cyclobutyl)methyl)amino)-2-(2,6- dioxopiperidin-3-yl)isoindoline-1,3-dione |
| GS-293 | 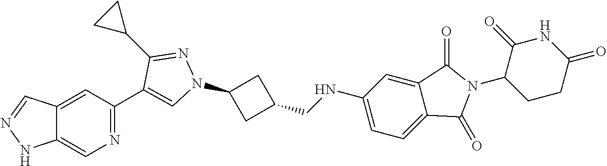 [SMILES: O=C1CCC(N2C(=O)c3ccc(NC[C@H]4C[C@H](n5cc(-c6cc7cn[nH]c7cn6)c(C6CC6)n5)C4)cc3C2=O)C(=O)N1] | 5-(((trans-3-(3-cyclopropyl-4-(1H- pyrazolo[3,4-c]pyridin-5-yl)-1H-pyrazol- 1-yl)cyclobutyl)methyl)amino)-2-(2,6- dioxopiperidin-3-yl)isoindoline-1,3-dione |
| GS-294 | 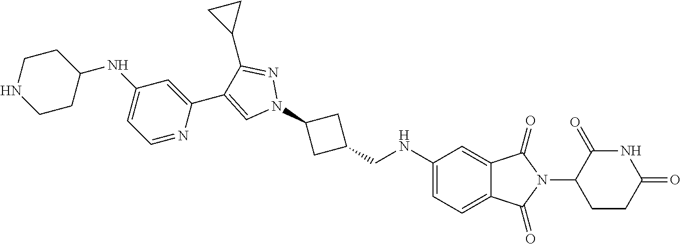 [SMILES: O=C1CCC(N2C(=O)c3ccc(NC[C@H]4C[C@H](n5cc(-c6cc(NC7CCNCC7)ccn6)c(C6CC6)n5)C4)cc3C2=O)C(=O)N1] | 5-(((trans-3-(3-cyclopropyl-4-(4- (piperidin-4-ylamino)pyridin-2-yl)-1H- pyrazol-1-yl)cyclobutyl)methyl)amino)- 2-(2,6-dioxopiperidin-3-yl)isoindoline- 1,3-dione |
| GS-295 | 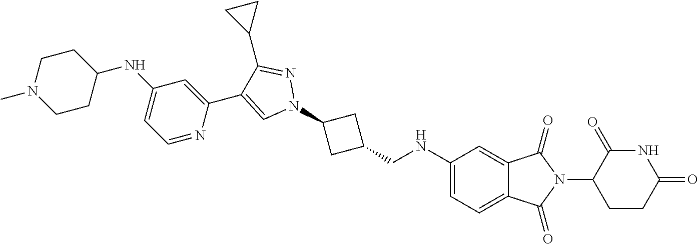 [SMILES: CN1CCC(Nc2ccnc(-c3cn([C@H]4C[C@H](CNc5ccc6c(c5)C(=O)N(C5CCC(=O)NC5=O)C6=O)C4)nc3C3CC3)c2)CC1] | 5-(((trans-3-(3-cyclopropyl-4-(4-((1- methylpiperidin-4-yl)amino)pyridin-2- yl)-1H-pyrazol-1- yl)cyclobutyl)methyl)amino)-2-(2,6- dioxopiperidin-3-yl)isoindoline-1,3-dione |
| GS-296 | 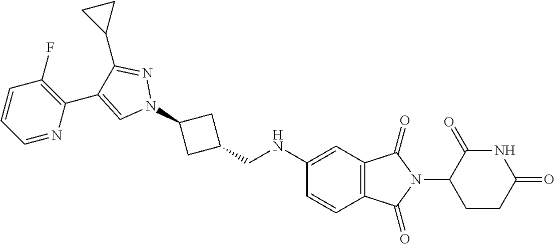 [SMILES: O=C1CCC(N2C(=O)c3ccc(NC[C@H]4C[C@H](n5cc(-c6ncccc6F)c(C6CC6)n5)C4)cc3C2=O)C(=O)N1] | 5-(((trans-3-(3-cyclopropyl-4-(3- fluoropyridin-2-yl)-1H-pyrazol-1- yl)cyclobutyl)methyl)amino)-2-(2,6- dioxopiperidin-3-yl)isoindoline-1,3-dione |
| GS-297 | 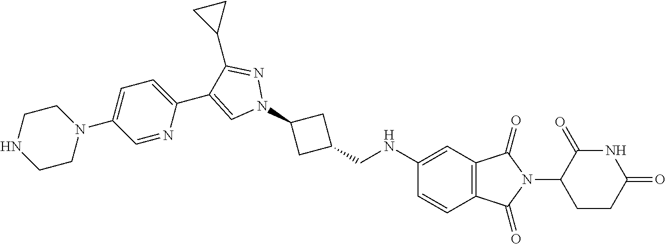 [SMILES: O=C1CCC(N2C(=O)c3ccc(NC[C@H]4C[C@H](n5cc(-c6ccc(N7CCNCC7)cn6)c(C6CC6)n5)C4)cc3C2=O)C(=O)N1] | 5-(((trans-3-(3-cyclopropyl-4-(5- (piperazin-1-yl)pyridin-2-yl)-1H-pyrazol- 1-yl)cyclobutyl)methyl)amino)-2-(2,6- dioxopiperidin-3-yl)isoindoline-1,3-dione |
| GS-298 | 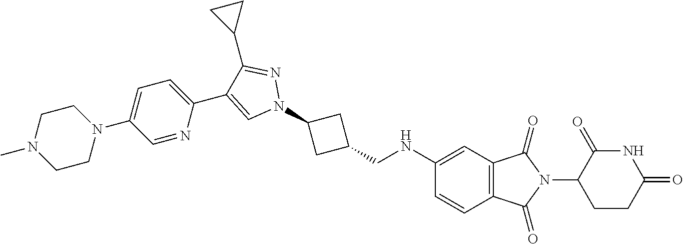 [SMILES: CN1CCN(c2ccc(-c3cn([C@H]4C[C@H](CNc5ccc6c(c5)C(=O)N(C5CCC(=O)NC5=O)C6=O)C4)nc3C3CC3)nc2)CC1] | 5-(((trans-3-(3-cyclopropyl-4-(5-(4- methylpiperazin-1-yl)pyridin-2-yl)-1H- pyrazol-1-yl)cyclobutyl)methyl)amino)- 2-(2,6-dioxopiperidin-3-yl)isoindoline- 1,3-dione |
| GS-299 | 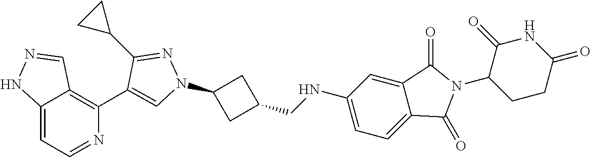 [SMILES: O=C1CCC(N2C(=O)c3ccc(NC[C@H]4C[C@H](n5cc(-c6nccc7[nH]ncc67)c(C6CC6)n5)C4)cc3C2=O)C(=O)N1] | 5-(((trans-3-(3-cyclopropyl-4-(1H- pyrazolo[4,3-c]pyridin-4-yl)-1H-pyrazol- 1-yl)cyclobutyl)methyl)amino)-2-(2,6- dioxopiperidin-3-yl)isoindoline-1,3-dione |
| GS-300 | 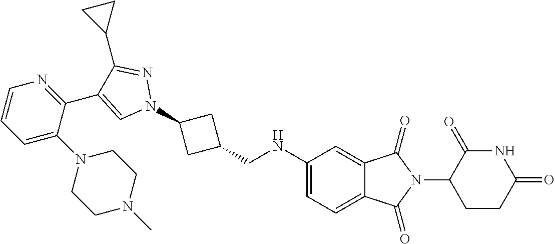 [SMILES: CN1CCN(c2cccnc2-c2cn([C@H]3C[C@H](CNc4ccc5c(c4)C(=O)N(C4CCC(=O)NC4=O)C5=O)C3)nc2C2CC2)CC1] | 5-(((trans-3-(4-cyclopropyl-3-(3-(4- methylpiperazin-1-yl)pyridin-2-yl)-1H- pyrazol-1-yl)cyclobutyl)methyl)amino)- 2-(2,6-dioxopiperidin-3-yl)isoindoline- 1,3-dione |
| GS-301 | 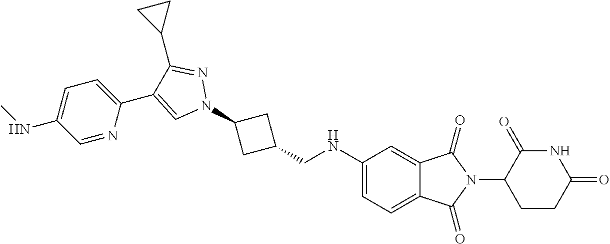 [SMILES: CNc1ccc(-c2cn([C@H]3C[C@H](CNc4ccc5c(c4)C(=O)N(C4CCC(=O)NC4=O)C5=O)C3)nc2C2CC2)nc1] | 5-(((trans-3-(3-cyclopropyl-4-(5- (methylamino)pyridin-2-yl)-1H-pyrazol- 1-yl)cyclobutyl)methyl)amino)-2-(2,6- dioxopiperidin-3-yl)isoindoline-1,3-dione |
| GS-302 | 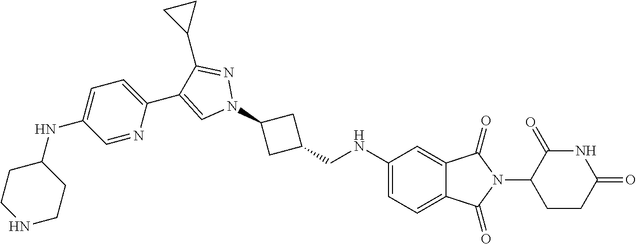 [SMILES: O=C1CCC(N2C(=O)c3ccc(NC[C@H]4C[C@H](n5cc(-c6ccc(NC7CCNCC7)cn6)c(C6CC6)n5)C4)cc3C2=O)C(=O)N1] | 5-(((trans-3-(3-cyclopropyl-4-(5- (piperidin-4-ylamino)pyridin-2-yl)-1H- pyrazol-1-yl)cyclobutyl)methyl)amino)- 2-(2,6-dioxopiperidin-3-yl)isoindoline- 1,3-dione |
| GS-303 | 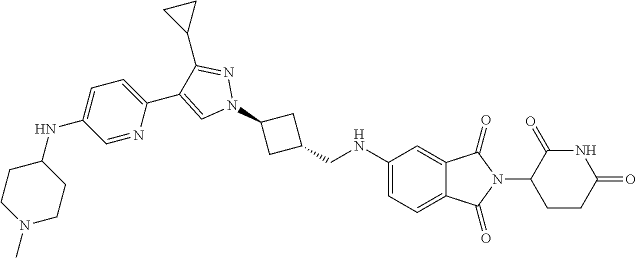 [SMILES: CN1CCC(Nc2ccc(-c3cn([C@H]4C[C@H](CNc5ccc6c(c5)C(=O)N(C5CCC(=O)NC5=O)C6=O)C4)nc3C3CC3)nc2)CC1] | 5-(((trans-3-(3-cyclopropyl-4-(5-((1- methylpiperidin-4-yl)amino)pyridin-2- yl)-1H-pyrazol-1- yl)cyclobutyl)methyl)amino)-2-(2,6- dioxopiperidin-3-yl)isoindoline-1,3-dione |
| GS-304 | 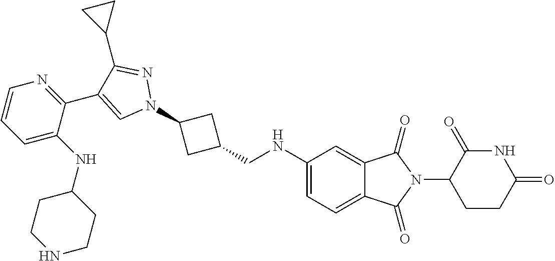 [SMILES: O=C1CCC(N2C(=O)c3ccc(NC[C@H]4C[C@H](n5cc(-c6ncccc6NC6CCNCC6)c(C6CC6)n5)C4)cc3C2=O)C(=O)N1] | 5-(((trans-3-(3-cyclopropyl-4-(3- (piperidin-4-ylamino)pyridin-2-yl)-1H- pyrazol-1-yl)cyclobutyl)methyl)amino)- 2-(2,6-dioxopiperidin-3-yl)isoindoline- 1,3-dione |
| GS-305 | 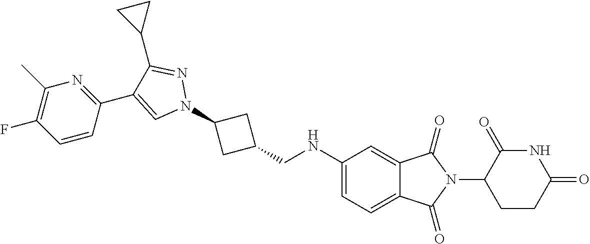 [SMILES: Cc1nc(-c2cn([C@H]3C[C@H](CNc4ccc5c(c4)C(=O)N(C4CCC(=O)NC4=O)C5=O)C3)nc2C2CC2)ccc1F] | 5-(((trans-3-(3-cyclopropyl-4-(5-fluoro- 6-methylpyridin-2-yl)-1H-pyrazol-1- yl)cyclobutyl)methyl)amino)-2-(2,6- dioxopiperidin-3-yl)isoindoline-1,3-dione |
| GS-306 | 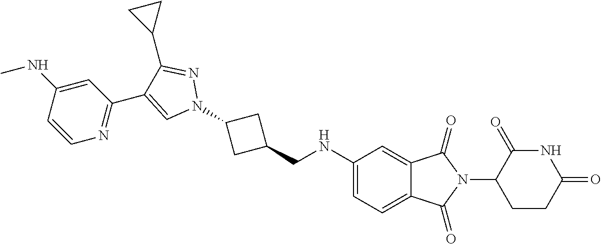 [SMILES: CNc1ccnc(-c2cn([C@H]3C[C@H](CNc4ccc5c(c4)C(=O)N(C4CCC(=O)NC4=O)C5=O)C3)nc2C2CC2)c1] | 5-(((trans-3-(3-cyclopropyl-4-(4- (methylamino)pyridin-2-yl)-1H-pyrazol- 1-yl)cyclobutyl)methyl)amino)-2-(2,6- dioxopiperidin-3-yl)isoindoline-1,3-dione |
| GS-307 | 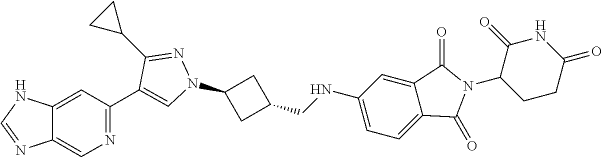 [SMILES: O=C1CCC(N2C(=O)c3ccc(NC[C@H]4C[C@H](n5cc(-c6cc7[nH]cnc7cn6)c(C6CC6)n5)C4)cc3C2=O)C(=O)N1] | 5-(((trans-3-(3-cyclopropyl-4-(1H- imidazo[4,5-c]pyridin-6-yl)-1H-pyrazol- 1-yl)cyclobutyl)methyl)amino)-2-(2,6- dioxopiperidin-3-yl)isoindoline-1,3-dione |
| GS-308 | 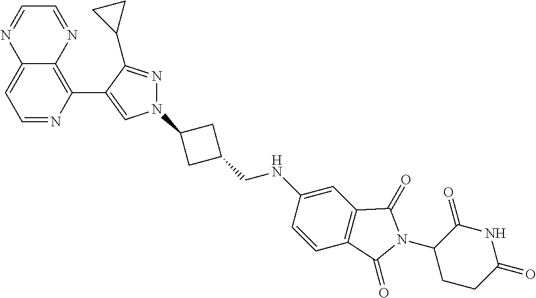 [SMILES: O=C1CCC(N2C(=O)c3ccc(NC[C@H]4C[C@H](n5cc(-c6nccc7nccnc67)c(C6CC6)n5)C4)cc3C2=O)C(=O)N1] | 5-(((trans-3-(3-cyclopropyl-4-(5,6,7,8- tetrahydroquinolin-2-yl)-1H-pyrazol-1- yl)cyclobutyl)methyl)amino)-2-(2,6- dioxopiperidin-3-yl)isoindoline-1,3-dione |
| GS-309 | 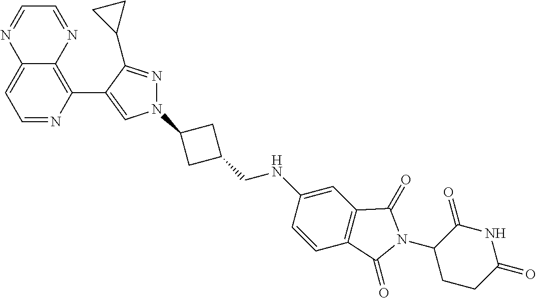 [SMILES: O=C1CCC(N2C(=O)c3ccc(NC[C@H]4C[C@H](n5cc(-c6nccc7nccnc67)c(C6CC6)n5)C4)cc3C2=O)C(=O)N1] | 5-(((trans-3-(3-cyclopropyl-4- (pyrido[3,4-b]pyrazin-5-yl)-1H-pyrazol- 1-yl)cyclobutyl)methyl)amino)-2-(2,6- dioxopiperidin-3-yl)isoindoline-1,3-dione |
| GS-310 | 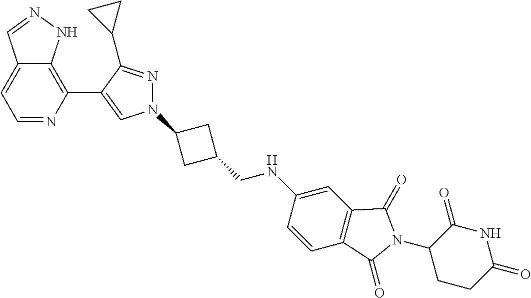 [SMILES: O=C1CCC(N2C(=O)c3ccc(NC[C@H]4C[C@H](n5cc(-c6nccc7cn[nH]c67)c(C6CC6)n5)C4)cc3C2=O)C(=O)N1] | 5-(((trans-3-(3-cyclopropyl-4-(1H- pyrazolo[3,4-c]pyridin-7-yl)-1H-pyrazol- 1-yl)cyclobutyl)methyl)amino)-2-(2,6- dioxopiperidin-3-yl)isoindoline-1,3-dione |
| GS-311 | 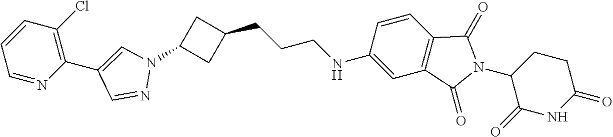 [SMILES: O=C1CCC(N2C(=O)c3ccc(NCCC[C@H]4C[C@H](n5cc(-c6ncccc6Cl)cn5)C4)cc3C2=O)C(=O)N1] | 5-((3-(trans-3-(4-(3-chloropyridin-2-yl)- 1H-pyrazol-1- yl)cyclobutyl)propyl)amino)-2-(2,6- dioxopiperidin-3-yl)isoindoline-1,3-dione |
| GS-312 | 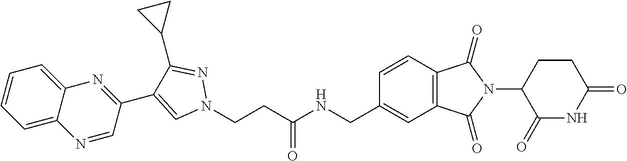 [SMILES: O=C(CCn1cc(-c2cnc3ccccc3n2)c(C2CC2)n1)NCc1ccc2c(c1)C(=O)N(C1CCC(=O)NC1=O)C2=O] | 3-(3-cyclopropyl-4-(quinoxalin-2-yl)-1H- pyrazol-1-yl)-N-((2-(2,6-dioxopiperidin- 3-yl)-1,3-dioxoisoindolin-5- yl)methyl)propanamide |
| GS-313 | 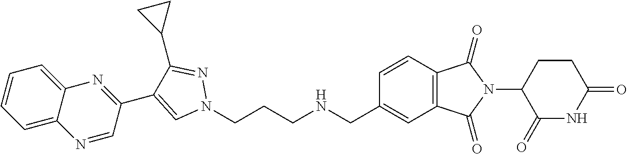 [SMILES: O=C1CCC(N2C(=O)c3ccc(CNCCCn4cc(-c5cnc6ccccc6n5)c(C5CC5)n4)cc3C2=O)C(=O)N1] | 5-(((3-(3-cyclopropyl-4-(quinoxalin-2- yl)-1H-pyrazol-1- yl)propyl)amino)methyl)-2-(2,6- dioxopiperidin-3-yl)isoindoline-1,3-dione |
| GS-314 | 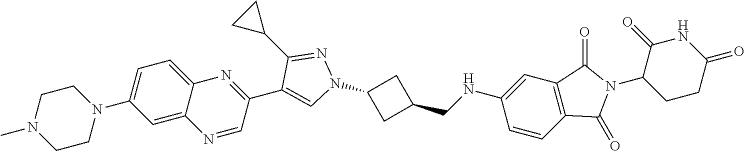 [SMILES: CN1CCN(c2ccc3nc(-c4cn([C@H]5C[C@H](CNc6ccc7c(c6)C(=O)N(C6CCC(=O)NC6=O)C7=O)C5)nc4C4CC4)cnc3c2)CC1] | 5-(((trans-3-(3-cyclopropyl-4-(6-(4- methylpiperazin-1-yl)quinoxalin-2-yl)- 1H-pyrazol-1- yl)cyclobutyl)methyl)amino)-2-(2,6- dioxopiperidin-3-yl)isoindoline-1,3-dione |
| GS-315 | 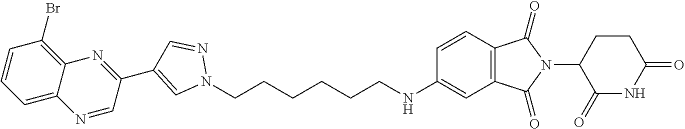 [SMILES: O=C1CCC(N2C(=O)c3ccc(NCCCCCCn4cc(-c5cnc6cccc(Br)c6n5)cn4)cc3C2=O)C(=O)N1] | 5-((6-(4-(8-bromoquinoxalin-2-yl)-1H- pyrazol-1-yl)hexyl)amino)-2-(2,6- dioxopiperidin-3-yl)isoindoline-1,3-dione |
| GS-316 | 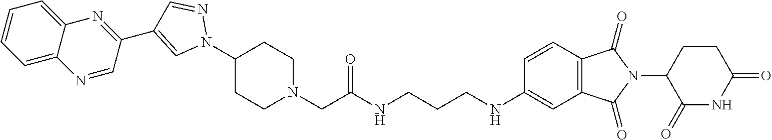 [SMILES: O=C(CN1CCC(n2cc(-c3cnc4ccccc4n3)cn2)CC1)NCCCNc1ccc2c(c1)C(=O)N(C1CCC(=O)NC1=O)C2=O] | N-(3-((2-(2,6-dioxopiperidin-3-yl)-1,3- dioxoisoindolin-5-yl)amino)propyl)-2-(4- (4-(quinoxalin-2-yl)-1H-pyrazol-1- yl)piperidin-1-yl)acetamide |
| GS-317 | 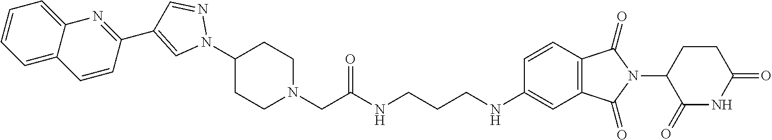 [SMILES: O=C(CN1CCC(n2cc(-c3ccc4ccccc4n3)cn2)CC1)NCCCNc1ccc2c(c1)C(=O)N(C1CCC(=O)NC1=O)C2=O] | N-(3-((2-(2,6-dioxopiperidin-3-yl)-1,3- dioxoisoindolin-5-yl)amino)propyl)-2-(4- (4-(quinolin-2-yl)-1H-pyrazol-1- yl)piperidin-1-yl)acetamide |
| GS-318 | 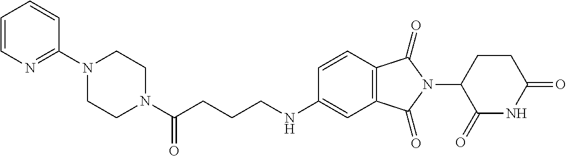 [SMILES: O=C1CCC(N2C(=O)c3ccc(NCCCC(=O)N4CCN(c5ccccn5)CC4)cc3C2=O)C(=O)N1] | 2-(2,6-dioxopiperidin-3-yl)-5-((4-oxo-4- (4-(pyridin-2-yl)piperazin-1- yl)butyl)amino)isoindoline-1,3-dione |
| GS-319 | 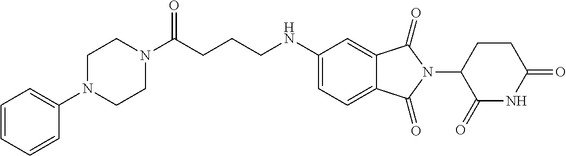 [SMILES: O=C1CCC(N2C(=O)c3ccc(NCCCC(=O)N4CCN(c5ccccc5)CC4)cc3C2=O)C(=O)N1] | 2-(2,6-dioxopiperidin-3-yl)-5-((4-oxo-4- (4-phenylpiperazin-1- yl)butyl)amino)isoindoline-1,3-dione |
| GS-320 | 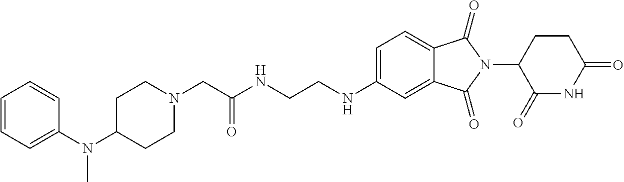 [SMILES: CN(c1ccccc1)C1CCN(CC(=O)NCCNc2ccc3c(c2)C(=O)N(C2CCC(=O)NC2=O)C3=O)CC1] | N-(2-((2-(2,6-dioxopiperidin-3-yl)-1,3- dioxoisoindolin-5-yl)amino)ethyl)-2-(4- (methyl(phenyl)amino)piperidin-1- yl)acetamide |
| GS-321 | 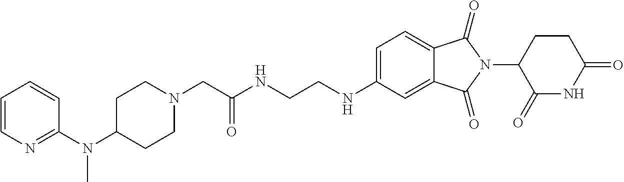 [SMILES: CN(c1ccccn1)C1CCN(CC(=O)NCCNc2ccc3c(c2)C(=O)N(C2CCC(=O)NC2=O)C3=O)CC1] | N-(2-((2-(2,6-dioxopiperidin-3-yl)-1,3- dioxoisoindolin-5-yl)amino)ethyl)-2-(4- (methyl(pyridin-2-yl)amino)piperidin-1- yl)acetamide |
| GS-322 | 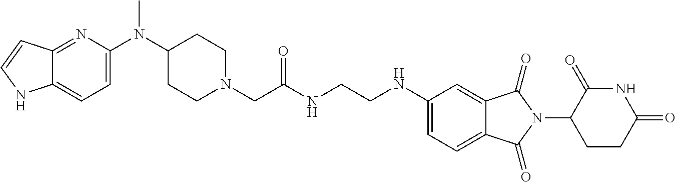 [SMILES: CN(c1ccc2[nH]ccc2n1)C1CCN(CC(=O)NCCNc2ccc3c(c2)C(=O)N(C2CCC(=O)NC2=O)C3=O)CC1] | N-(2-((2-(2,6-dioxopiperidin-3-yl)-1,3- dioxoisoindolin-5-yl)amino)ethyl)-2-(4- (methyl(1H-pyrrolo[3,2-b]pyridin-5- yl)amino)piperidin-1-yl)acetamide |
| GS-323 | 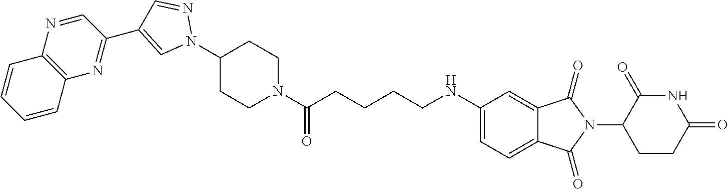 [SMILES: O=C1CCC(N2C(=O)c3ccc(NCCCCC(=O)N4CCC(n5cc(-c6cnc7ccccc7n6)cn5)CC4)cc3C2=O)C(=O)N1] | 2-(2,6-dioxopiperidin-3-yl)-5-((5-oxo-5- (4-(4-(quinoxalin-2-yl)-1H-pyrazol-1- yl)piperidin-1- yl)pentyl)amino)isoindoline-1,3-dione |
| GS-324 | 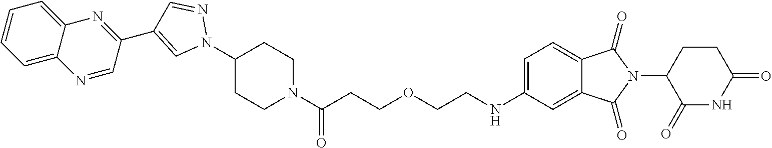 [SMILES: O=C1CCC(N2C(=O)c3ccc(NCCOCCC(=O)N4CCC(n5cc(-c6cnc7ccccc7n6)cn5)CC4)cc3C2=O)C(=O)N1] | 2-(2,6-dioxopiperidin-3-yl)-5-((2-(3-oxo- 3-(4-(4-(quinoxalin-2-yl)-1H-pyrazol-1- yl)piperidin-1- yl)propoxy)ethyl)amino)isoindoline-1,3- dione |
| GS-325 | 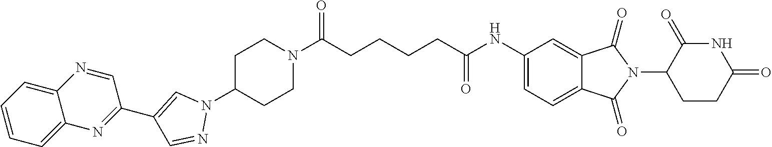 [SMILES: O=C1CCC(N2C(=O)c3ccc(NC(=O)CCCCC(=O)N4CCC(n5cc(-c6cnc7ccccc7n6)cn5)CC4)cc3C2=O)C(=O)N1] | N-(2-(2,6-dioxopiperidin-3-yl)-1,3- dioxoisoindolin-5-yl)-6-oxo-6-(4-(4- (quinoxalin-2-yl)-1H-pyrazol-1- yl)piperidin-1-yl)hexanamide |
| GS-326 | 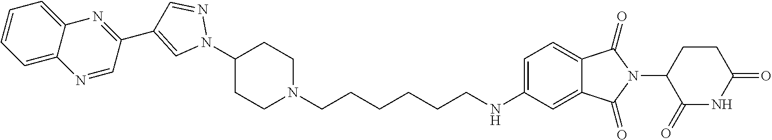 [SMILES: O=C1CCC(N2C(=O)c3ccc(NCCCCCCN4CCC(n5cc(-c6cnc7ccccc7n6)cn5)CC4)cc3C2=O)C(=O)N1] | 2-(2,6-dioxopiperidin-3-yl)-5-((6-(4-(4- (quinoxalin-2-yl)-1H-pyrazol-1- yl)piperidin-1- yl)hexyl)amino)isoindoline-1,3-dione |
| GS-327 | 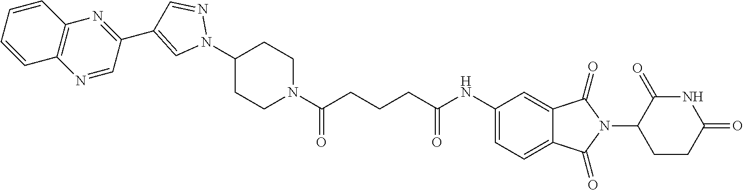 [SMILES: O=C1CCC(N2C(=O)c3ccc(NC(=O)CCCC(=O)N4CCC(n5cc(-c6cnc7ccccc7n6)cn5)CC4)cc3C2=O)C(=O)N1] | N-(2-(2,6-dioxopiperidin-3-yl)-1,3- dioxoisoindolin-5-yl)-5-oxo-5-(4-(4- (quinoxalin-2-yl)-1H-pyrazol-1- yl)piperidin-1-yl)pentanamide |
| GS-328 | 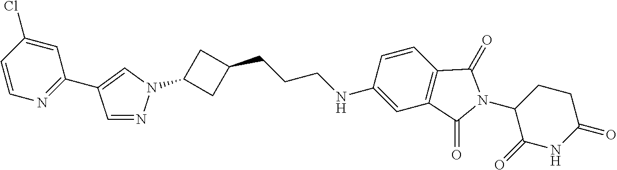 [SMILES: O=C1CCC(N2C(=O)c3ccc(NCCC[C@H]4C[C@H](n5cc(-c6cc(Cl)ccn6)cn5)C4)cc3C2=O)C(=O)N1] | 5-((3-(trans-3-(4-(4-chloropyridin-2-yl)- 1H-pyrazol-1- yl)cyclobutyl)propyl)amino)-2-(2,6- dioxopiperidin-3-yl)isoindoline-1,3-dione |
| GS-329 | 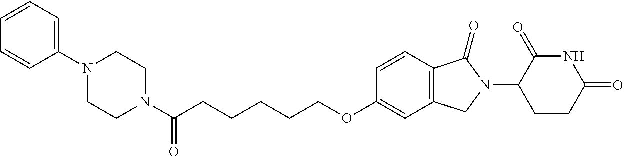 [SMILES: O=C1CCC(N2Cc3cc(OCCCCCC(=O)N4CCN(c5ccccc5)CC4)ccc3C2=O)C(=O)N1] | 3-(1-oxo-5-((6-oxo-6-(4-phenylpiperazin- 1-yl)hexyl)oxy)isoindolin-2- yl)piperidine-2,6-dione |
| GS-330 | 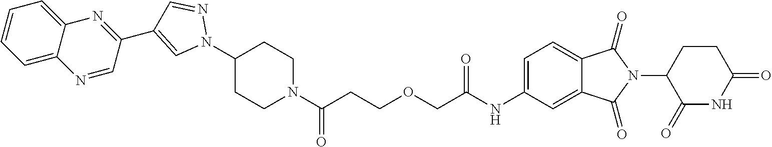 [SMILES: O=C1CCC(N2C(=O)c3ccc(NC(=O)COCCC(=O)N4CCC(n5cc(-c6cnc7ccccc7n6)cn5)CC4)cc3C2=O)C(=O)N1] | N-(2-(2,6-dioxopiperidin-3-yl)-1,3- dioxoisoindolin-5-yl)-2-(3-oxo-3-(4-(4- (quinoxalin-2-yl)-1H-pyrazol-1- yl)piperidin-1-yl)propoxy)acetamide |
| GS-331 | 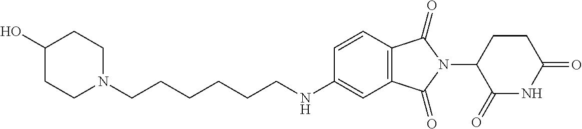 [SMILES: O=C1CCC(N2C(=O)c3ccc(NCCCCCCN4CCC(O)CC4)cc3C2=O)C(=O)N1] | 2-(2,6-dioxopiperidin-3-yl)-5-((6-(4- hydroxypiperidin-1- yl)hexyl)amino)isoindoline-1,3-dione |
| GS-332 | 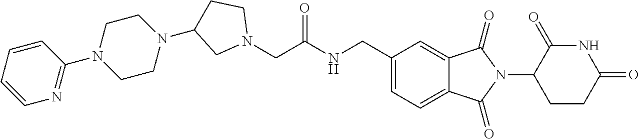 [SMILES: O=C(CN1CCC(N2CCN(c3ccccn3)CC2)C1)NCc1ccc2c(c1)C(=O)N(C1CCC(=O)NC1=O)C2=O] | N-((2-(2,6-dioxopiperidin-3-yl)-1,3- dioxoisoindolin-5-yl)methyl)-2-(3-(4- (pyridin-2-yl)piperazin-1-yl)pyrrolidin-1- yl)acetamide |
| GS-333 | 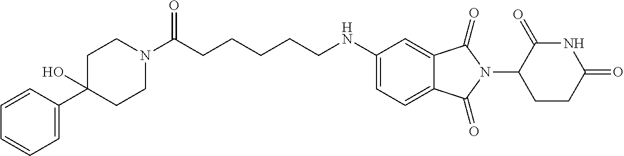 [SMILES: O=C1CCC(N2C(=O)c3ccc(NCCCCCC(=O)N4CCC(O)(c5ccccc5)CC4)cc3C2=O)C(=O)N1] | 2-(2,6-dioxopiperidin-3-yl)-5-((6-(4- hydroxy-4-phenylpiperidin-1-yl)-6- oxohexyl)amino)isoindoline-1,3-dione |
| GS-334 | 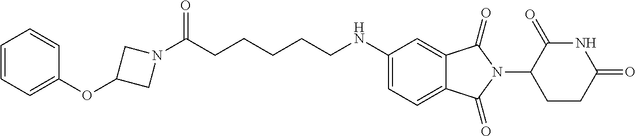 [SMILES: O=C1CCC(N2C(=O)c3ccc(NCCCCCC(=O)N4CC(Oc5ccccc5)C4)cc3C2=O)C(=O)N1] | 2-(2,6-dioxopiperidin-3-yl)-5-((6-oxo-6- (3-phenoxyazetidin-1- yl)hexyl)amino)isoindoline-1,3-dione |
| GS-335 | 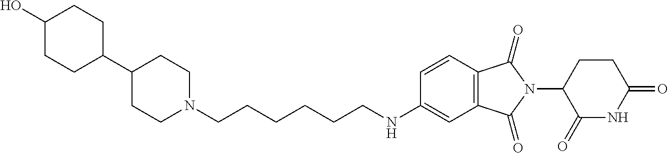 [SMILES: O=C1CCC(N2C(=O)c3ccc(NCCCCCCN4CCC(C5CCC(O)CC5)CC4)cc3C2=O)C(=O)N1] | 2-(2,6-dioxopiperidin-3-yl)-5-((6-(4-(4- hydroxycyclohexyl)piperidin-1- yl)hexyl)amino)isoindoline-1,3-dione |
| GS-336 | 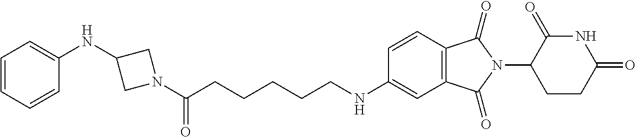 [SMILES: O=C1CCC(N2C(=O)c3ccc(NCCCCCC(=O)N4CC(Nc5ccccc5)C4)cc3C2=O)C(=O)N1] | 2-(2,6-dioxopiperidin-3-yl)-5-((6-oxo-6- (3-(phenylamino)azetidin-1- yl)hexyl)amino)isoindoline-1,3-dione |
| GS-337 | 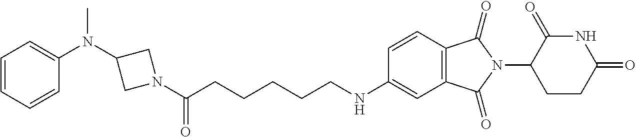 [SMILES: CN(c1ccccc1)C1CN(C(=O)CCCCCNc2ccc3c(c2)C(=O)N(C2CCC(=O)NC2=O)C3=O)C1] | 2-(2,6-dioxopiperidin-3-yl)-5-((6-(3- (methyl(phenyl)amino)azetidin-1-yl)-6- oxohexyl)amino)isoindoline-1,3-dione |
| GS-338 | 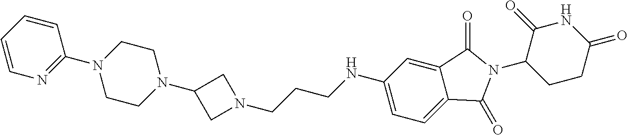 [SMILES: O=C1CCC(N2C(=O)c3ccc(NCCCN4CC(N5CCN(c6ccccn6)CC5)C4)cc3C2=O)C(=O)N1] | 2-(2,6-dioxopiperidin-3-yl)-5-((3-(3-(4- (pyridin-2-yl)piperazin-1-yl)azetidin-1- yl)propyl)amino)isoindoline-1,3-dione |
| GS-339 | 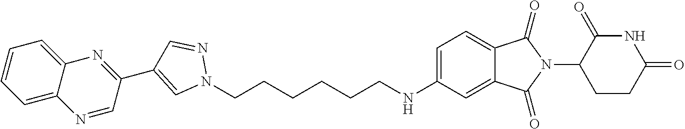 [SMILES: O=C1CCC(N2C(=O)c3ccc(NCCCCCCn4cc(-c5cnc6ccccc6n5)cn4)cc3C2=O)C(=O)N1] | 2-(2,6-dioxopiperidin-3-yl)-5-((6-(4- (quinoxalin-2-yl)-1H-pyrazol-1- yl)hexyl)amino)isoindoline-1,3-dione |
| GS-340 | 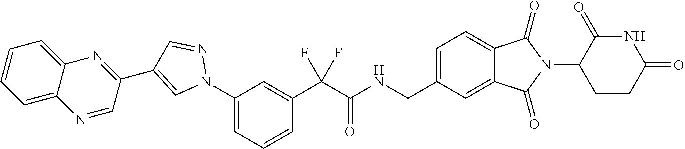 [SMILES: O=C1CCC(N2C(=O)c3ccc(CNC(=O)C(F)(F)c4cccc(-n5cc(-c6cnc7ccccc7n6)cn5)c4)cc3C2=O)C(=O)N1] | N-((2-(2,6-dioxopiperidin-3-yl)-1,3- dioxoisoindolin-5-yl)methyl)-2,2- difluoro-2-(3-(4-(quinoxalin-2-yl)-1H- pyrazol-1-yl)phenyl)acetamide |
| GS-341 | 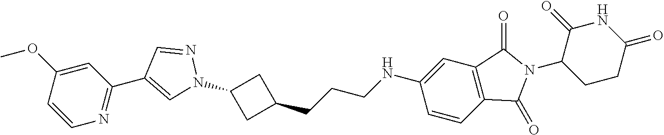 [SMILES: COc1ccnc(-c2cnn([C@H]3C[C@H](CCCNc4ccc5c(c4)C(=O)N(C4CCC(=O)NC4=O)C5=O)C3)c2)c1] | 2-(2,6-dioxopiperidin-3-yl)-5-((3-(trans- 3-(4-(4-methoxypyridin-2-yl)-1H- pyrazol-1- yl)cyclobutyl)propyl)amino)isoindoline- 1,3-dione |
| GS-342 | 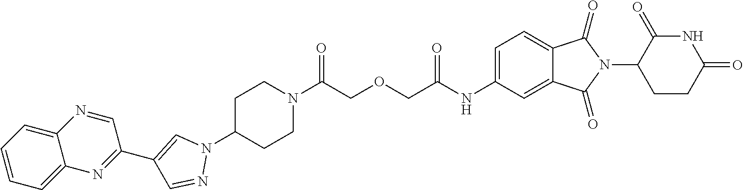 [SMILES: O=C1CCC(N2C(=O)c3ccc(NC(=O)COCC(=O)N4CCC(n5cc(-c6cnc7ccccc7n6)cn5)CC4)cc3C2=O)C(=O)N1] | N-(2-(2,6-dioxopiperidin-3-yl)-1,3- dioxoisoindolin-5-yl)-2-(2-oxo-2-(4-(4- (quinoxalin-2-yl)-1H-pyrazol-1- yl)piperidin-1-yl)ethoxy)acetamide |
| GS-343 | 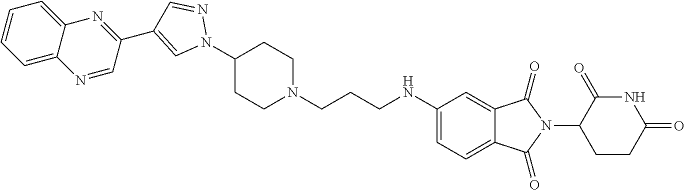 [SMILES: O=C1CCC(N2C(=O)c3ccc(NCCCN4CCC(n5cc(-c6cnc7ccccc7n6)cn5)CC4)cc3C2=O)C(=O)N1] | 2-(2,6-dioxopiperidin-3-yl)-5-((3-(4-(4- (quinoxalin-2-yl)-1H-pyrazol-1- yl)piperidin-1- yl)propyl)amino)isoindoline-1,3-dione |
| GS-344 | 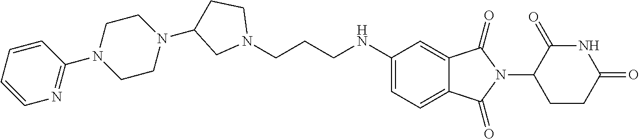 [SMILES: O=C1CCC(N2C(=O)c3ccc(NCCCN4CCC(N5CCN(c6ccccn6)CC5)C4)cc3C2=O)C(=O)N1] | 2-(2,6-dioxopiperidin-3-yl)-5-((3-(3-(4- (pyridin-2-yl)piperazin-1-yl)pyrrolidin-1- yl)propyl)amino)isoindoline-1,3-dione |
| GS-345 | 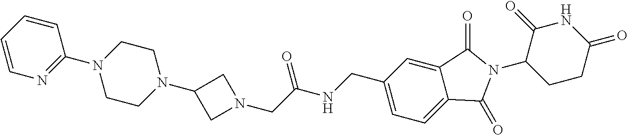 [SMILES: O=C(CN1CC(N2CCN(c3ccccn3)CC2)C1)NCc1ccc2c(c1)C(=O)N(C1CCC(=O)NC1=O)C2=O] | N-((2-(2,6-dioxopiperidin-3-yl)-1,3- dioxoisoindolin-5-yl)methyl)-2-(3-(4- (pyridin-2-yl)piperazin-1-yl)azetidin-1- yl)acetamide |
| GS-346 | 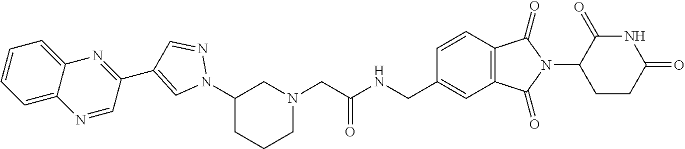 [SMILES: O=C(CN1CCCC(n2cc(-c3cnc4ccccc4n3)cn2)C1)NCc1ccc2c(c1)C(=O)N(C1CCC(=O)NC1=O)C2=O] | N-((2-(2,6-dioxopiperidin-3-yl)-1,3- dioxoisoindolin-5-yl)methyl)-2-(3-(4- (quinoxalin-2-yl)-1H-pyrazol-1- yl)piperidin-1-yl)acetamide |
| GS-347 | 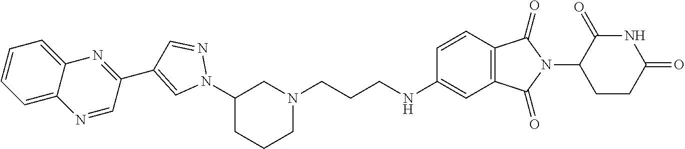 [SMILES: O=C1CCC(N2C(=O)c3ccc(NCCCN4CCCC(n5cc(-c6cnc7ccccc7n6)cn5)C4)cc3C2=O)C(=O)N1] | 2-(2,6-dioxopiperidin-3-yl)-5-((3-(3-(4- (quinoxalin-2-yl)-1H-pyrazol-1- yl)piperidin-1- yl)propyl)amino)isoindoline-1,3-dione |
| GS-348 | 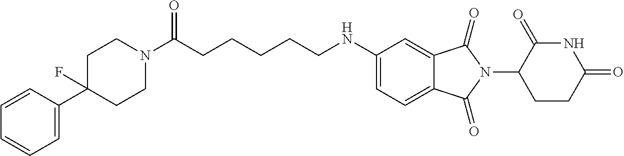 [SMILES: O=C1CCC(N2C(=O)c3ccc(NCCCCCC(=O)N4CCC(F)(c5ccccc5)CC4)cc3C2=O)C(=O)N1] | 2-(2,6-dioxopiperidin-3-yl)-5-((6-(4- fluoro-4-phenylpiperidin-1-yl)-6- oxohexyl)amino)isoindoline-1,3-dione |
| GS-349 | 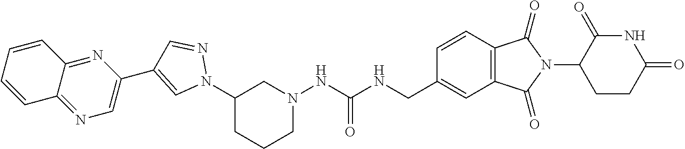 [SMILES: O=C1CCC(N2C(=O)c3ccc(CNC(=O)NN4CCCC(n5cc(-c6cnc7ccccc7n6)cn5)C4)cc3C2=O)C(=O)N1] | 1-((2-(2,6-dioxopiperidin-3-yl)-1,3- dioxoisoindolin-5-yl)methyl)-3-(3-(4- (quinoxalin-2-yl)-1H-pyrazol-1- yl)cyclohexyl)urea |
| GS-350 | 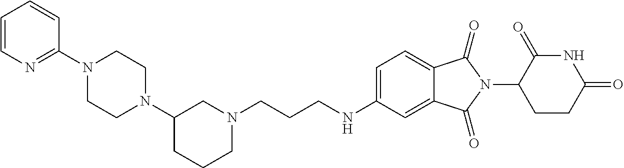 [SMILES: O=C1CCC(N2C(=O)c3ccc(NCCCN4CCCC(N5CCN(c6ccccn6)CC5)C4)cc3C2=O)C(=O)N1] | 2-(2,6-dioxopiperidin-3-yl)-5-((3-(3-(4- (pyridin-2-yl)piperazin-1-yl)piperidin-1- yl)propyl)amino)isoindoline-1,3-dione |
| GS-351 | 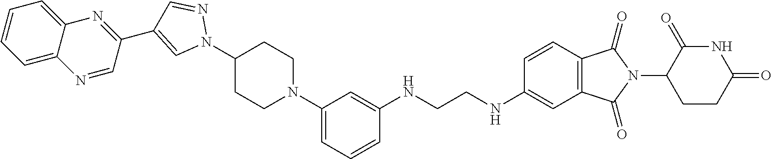 [SMILES: O=C1CCC(N2C(=O)c3ccc(NCCNc4cccc(N5CCC(n6cc(-c7cnc8ccccc8n7)cn6)CC5)c4)cc3C2=O)C(=O)N1] | 2-(2,6-dioxopiperidin-3-yl)-5-((2-((3-(4- (4-(quinoxalin-2-yl)-1H-pyrazol-1- yl)piperidin-1- yl)phenyl)amino)ethyl)amino)isoindoline- 1,3-dione |
| GS-352 | 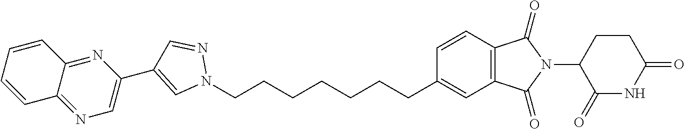 [SMILES: O=C1CCC(N2C(=O)c3ccc(CCCCCCCn4cc(-c5cnc6ccccc6n5)cn4)cc3C2=O)C(=O)N1] | 2-(2,6-dioxopiperidin-3-yl)-5-(7-(4- (quinoxalin-2-yl)-1H-pyrazol-1- yl)heptyl)isoindoline-1,3-dione |
| GS-353 | 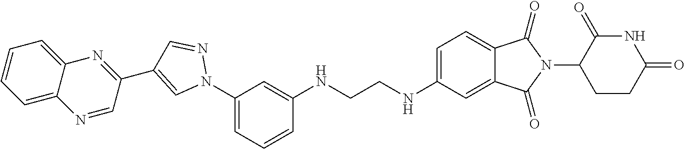 [SMILES: O=C1CCC(N2C(=O)c3ccc(NCCNc4cccc(-n5cc(-c6cnc7ccccc7n6)cn5)c4)cc3C2=O)C(=O)N1] | 2-(2,6-dioxopiperidin-3-yl)-5-((2-((3-(4- (quinoxalin-2-yl)-1H-pyrazol-1- yl)phenyl)amino)ethyl)amino)isoindoline- 1,3-dione |
| GS-354 | 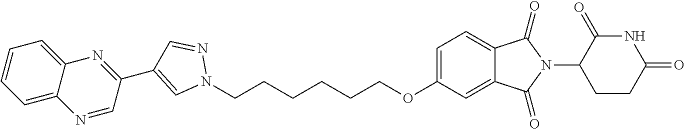 [SMILES: O=C1CCC(N2C(=O)c3ccc(OCCCCCCn4cc(-c5cnc6ccccc6n5)cn4)cc3C2=O)C(=O)N1] | 2-(2,6-dioxopiperidin-3-yl)-5-((6-(4- (quinoxalin-2-yl)-1H-pyrazol-1- yl)hexyl)oxy)isoindoline-1,3-dione |
| GS-355 | 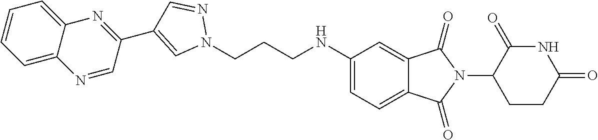 [SMILES: O=C1CCC(N2C(=O)c3ccc(NCCCn4cc(-c5cnc6ccccc6n5)cn4)cc3C2=O)C(=O)N1] | 2-(2,6-dioxopiperidin-3-yl)-5-((3-(4- (quinoxalin-2-yl)-1H-pyrazol-1- yl)propyl)amino)isoindoline-1,3-dione |
| GS-356 | 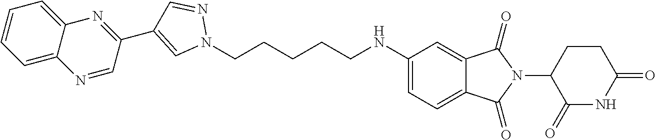 [SMILES: O=C1CCC(N2C(=O)c3ccc(NCCCCCn4cc(-c5cnc6ccccc6n5)cn4)cc3C2=O)C(=O)N1] | 2-(2,6-dioxopiperidin-3-yl)-5-((5-(4- (quinoxalin-2-yl)-1H-pyrazol-1- yl)pentyl)amino)isoindoline-1,3-dione |
| GS-357 | 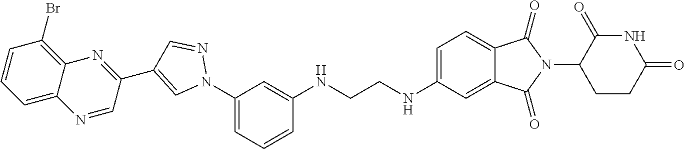 [SMILES: O=C1CCC(N2C(=O)c3ccc(NCCNc4cccc(-n5cc(-c6cnc7cccc(Br)c7n6)cn5)c4)cc3C2=O)C(=O)N1] | 5-((2-((3-(4-(8-bromoquinoxalin-2-yl)- 1H-pyrazol-1- yl)phenyl)amino)ethyl)amino)-2-(2,6- dioxopiperidin-3-yl)isoindoline-1,3-dione |
| GS-358 | 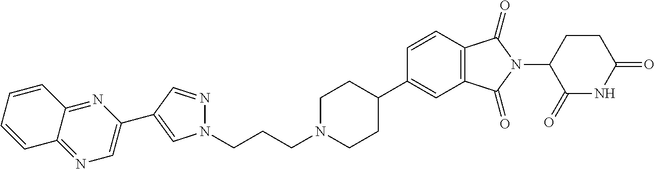 [SMILES: O=C1CCC(N2C(=O)c3ccc(C4CCN(CCCn5cc(-c6cnc7ccccc7n6)cn5)CC4)cc3C2=O)C(=O)N1] | 2-(2,6-dioxopiperidin-3-yl)-5-(1-(3-(4- (quinoxalin-2-yl)-1H-pyrazol-1- yl)propyl)piperidin-4-yl)isoindoline-1,3- dione |
| GS-359 | 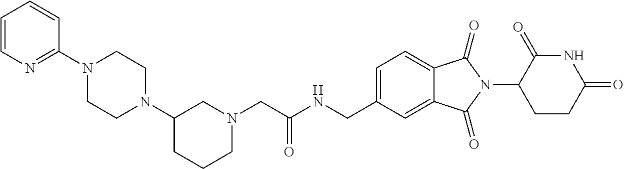 [SMILES: O=C(CN1CCCC(N2CCN(c3ccccn3)CC2)C1)NCc1ccc2c(c1)C(=O)N(C1CCC(=O)NC1=O)C2=O] | N-((2-(2,6-dioxopiperidin-3-yl)-1,3- dioxoisoindolin-5-yl)methyl)-2-(3-(4- (pyridin-2-yl)piperazin-1-yl)piperidin-1- yl)acetamide |
| GS-360 | 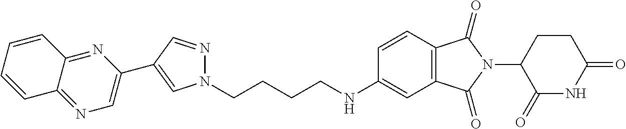 [SMILES: O=C1CCC(N2C(=O)c3ccc(NCCCCn4cc(-c5cnc6ccccc6n5)cn4)cc3C2=O)C(=O)N1] | 2-(2,6-dioxopiperidin-3-yl)-5-((4-(4- (quinoxalin-2-yl)-1H-pyrazol-1- yl)butyl)amino)isoindoline-1,3-dione |
| GS-361 | 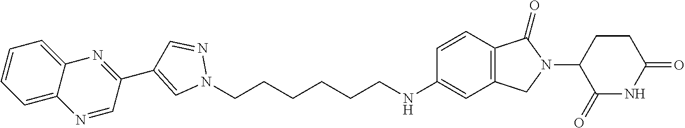 [SMILES: O=C1CCC(N2Cc3cc(NCCCCCCn4cc(-c5cnc6ccccc6n5)cn4)ccc3C2=O)C(=O)N1] | 3-(1-oxo-5-((6-(4-(quinoxalin-2-yl)-1H- pyrazol-1-yl)hexyl)amino)isoindolin-2- yl)piperidine-2,6-dione |
| GS-362 | 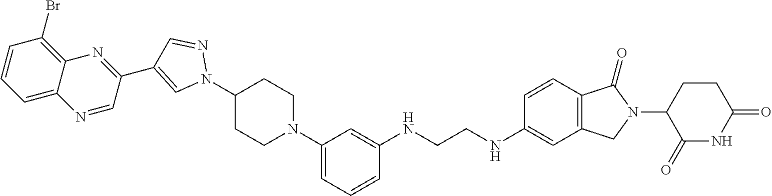 [SMILES: O=C1CCC(N2Cc3cc(NCCNc4cccc(N5CCC(n6cc(-c7cnc8cccc(Br)c8n7)cn6)CC5)c4)ccc3C2=O)C(=O)N1] | 5-((2-((3-(4-(4-(8-bromoquinoxalin-2-yl)- 1H-pyrazol-1-yl)piperidin-1- yl)phenyl)amino)ethyl)amino)-2-(2,6- dioxopiperidin-3-yl)isoindoline-1,3-dione |
| GS-363 | 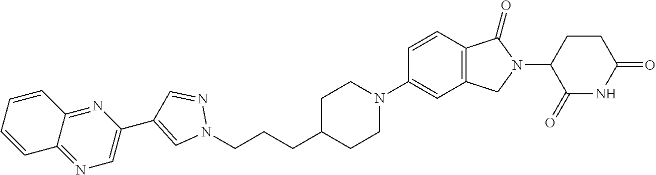 [SMILES: O=C1CCC(N2Cc3cc(N4CCC(CCCn5cc(-c6cnc7ccccc7n6)cn5)CC4)ccc3C2=O)C(=O)N1] | 2-(2,6-dioxopiperidin-3-yl)-5-(4-(3-(4- (quinoxalin-2-yl)-1H-pyrazol-1- yl)propyl)piperidin-1-yl)isoindoline-1,3- dione |
| GS-364 | 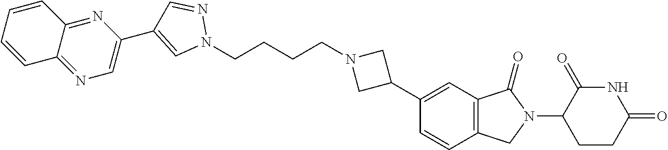 [SMILES: O=C1CCC(N2Cc3ccc(C4CN(CCCCn5cc(-c6cnc7ccccc7n6)cn5)C4)cc3C2=O)C(=O)N1] | 2-(2,6-dioxopiperidin-3-yl)-5-(1-(4-(4- (quinoxalin-2-yl)-1H-pyrazol-1- yl)butyl)azetidin-3-yl)isoindoline-1,3- dione |
| GS-365 | 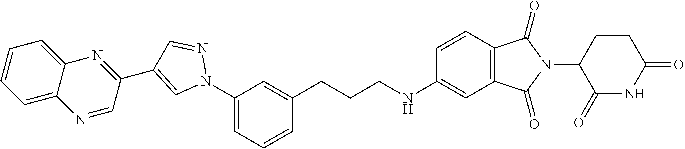 [SMILES: O=C1CCC(N2C(=O)c3ccc(NCCCc4cccc(-n5cc(-c6cnc7ccccc7n6)cn5)c4)cc3C2=O)C(=O)N1] | 2-(2,6-dioxopiperidin-3-yl)-5-((3-(3-(4- (quinoxalin-2-yl)-1H-pyrazol-1- yl)phenyl)propyl)amino)isoindoline-1,3- dione |
| GS-366 | 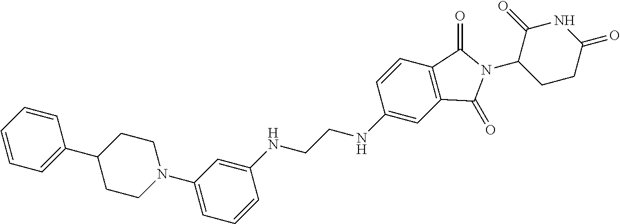 [SMILES: O=C1CCC(N2C(=O)c3ccc(NCCNc4cccc(N5CCC(c6ccccc6)CC5)c4)cc3C2=O)C(=O)N1] | 2-(2,6-dioxopiperidin-3-yl)-5-((2-((3-(4- phenylpiperidin-1- yl)phenyl)amino)ethyl)amino)isoindoline- 1,3-dione |
| GS-367 | 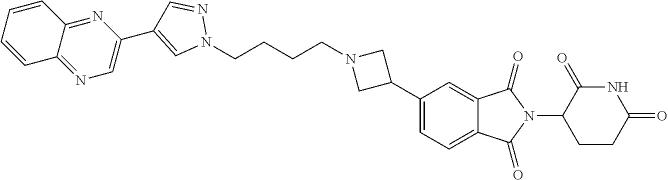 [SMILES: O=C1CCC(N2C(=O)c3ccc(C4CN(CCCCn5cc(-c6cnc7ccccc7n6)cn5)C4)cc3C2=O)C(=O)N1] | 2-(2,6-dioxopiperidin-3-yl)-5-(3-(4-(4- (quinoxalin-2-yl)-1H-pyrazol-1- yl)butyl)azetidin-1-yl)isoindoline-1,3- dione |
| GS-368 | 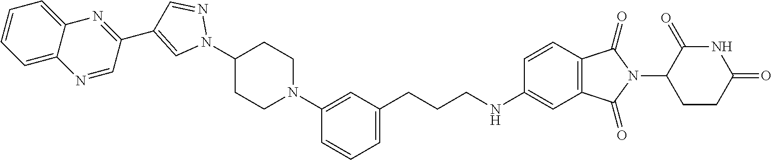 [SMILES: O=C1CCC(N2C(=O)c3ccc(NCCCc4cccc(N5CCC(n6cc(-c7cnc8ccccc8n7)cn6)CC5)c4)cc3C2=O)C(=O)N1] | 2-(2,6-dioxopiperidin-3-yl)-5-((3-(3-(4- (4-(quinoxalin-2-yl)-1H-pyrazol-1- yl)piperidin-1- yl)phenyl)propyl)amino)isoindoline-1,3- dione |
| GS-369 | 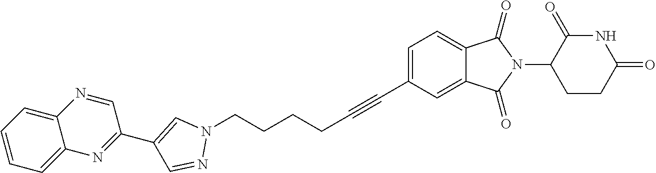 [SMILES: O=C1CCC(N2C(=O)c3ccc(C#CCCCCn4cc(-c5cnc6ccccc6n5)cn4)cc3C2=O)C(=O)N1] | 2-(2,6-dioxopiperidin-3-yl)-5-(6-(4- (quinoxalin-2-yl)-1H-pyrazol-1-yl)hex-1- yn-1-yl)isoindoline-1,3-dione |
| GS-370 | 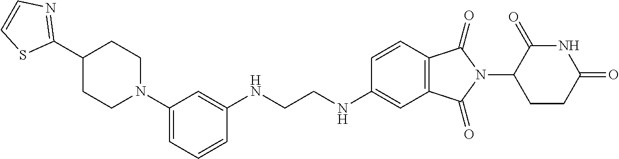 [SMILES: O=C1CCC(N2C(=O)c3ccc(NCCNc4cccc(N5CCC(c6nccs6)CC5)c4)cc3C2=O)C(=O)N1] | 2-(2,6-dioxopiperidin-3-yl)-5-((2-((3-(4- (thiazol-2-yl)piperidin-1- yl)phenyl)amino)ethyl)amino)isoindoline- 1,3-dione |
| GS-371 | 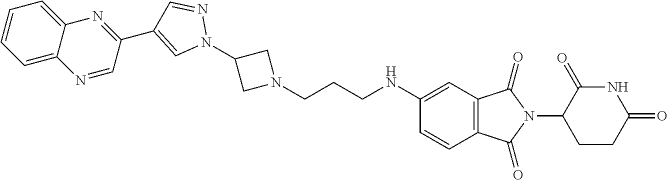 [SMILES: O=C1CCC(N2C(=O)c3ccc(NCCCN4CC(n5cc(-c6cnc7ccccc7n6)cn5)C4)cc3C2=O)C(=O)N1] | 2-(2,6-dioxopiperidin-3-yl)-5-((3-(3-(4- (quinoxalin-2-yl)-1H-pyrazol-1- yl)azetidin-1- yl)propyl)amino)isoindoline-1,3-dione |
| GS-372 | 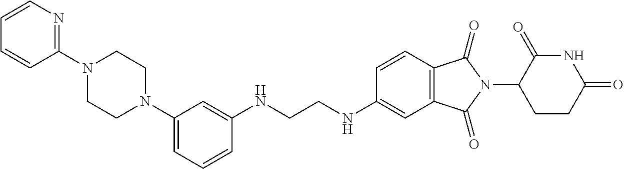 [SMILES: O=C1CCC(N2C(=O)c3ccc(NCCNc4cccc(N5CCN(c6ccccn6)CC5)c4)cc3C2=O)C(=O)N1] | 2-(2,6-dioxopiperidin-3-yl)-5-((2-((3-(4- (pyridin-2-yl)piperazin-1- yl)phenyl)amino)ethyl)amino)isoindoline- 1,3-dione |
| GS-373 | 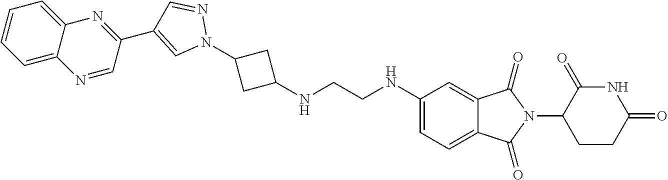 [SMILES: O=C1CCC(N2C(=O)c3ccc(NCCNC4CC(n5cc(-c6cnc7ccccc7n6)cn5)C4)cc3C2=O)C(=O)N1] | 2-(2,6-dioxopiperidin-3-yl)-5-((2-((3-(4- (quinoxalin-2-yl)-1H-pyrazol-1- yl)cyclobutyl)amino)ethyl)amino)iso- indoline-1,3-dione |
| GS-374 | 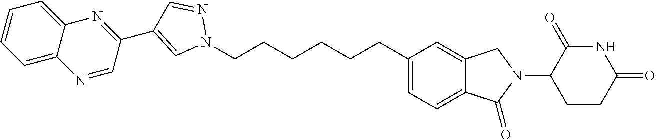 [SMILES: O=C1CCC(N2Cc3cc(CCCCCCn4cc(-c5cnc6ccccc6n5)cn4)ccc3C2=O)C(=O)N1] | 3-(1-oxo-5-(6-(4-(quinoxalin-2-yl)-1H- pyrazol-1-yl)hexyl)isoindolin-2- yl)piperidine-2,6-dione |
| GS-375 | 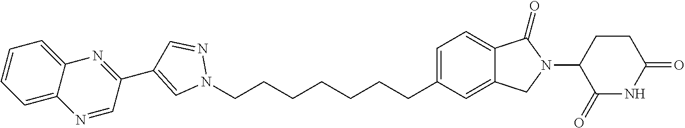 [SMILES: O=C1CCC(N2Cc3cc(CCCCCCCn4cc(-c5cnc6ccccc6n5)cn4)ccc3C2=O)C(=O)N1] | 3-(1-oxo-5-(7-(4-(quinoxalin-2-yl)-1H- pyrazol-1-yl)heptyl)isoindolin-2- yl)piperidine-2,6-dione |
| GS-376 | 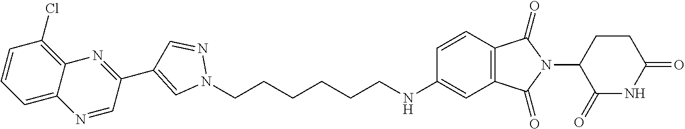 [SMILES: O=C1CCC(N2C(=O)c3ccc(NCCCCCCn4cc(-c5cnc6cccc(Cl)c6n5)cn4)cc3C2=O)C(=O)N1] | 5-((6-(4-(8-chloroquinoxalin-2-yl)-1H- pyrazol-1-yl)hexyl)amino)-2-(2,6- dioxopiperidin-3-yl)isoindoline-1,3-dione |
| GS-377 | 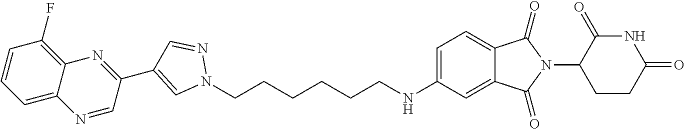 [SMILES: O=C1CCC(N2C(=O)c3ccc(NCCCCCCn4cc(-c5cnc6cccc(F)c6n5)cn4)cc3C2=O)C(=O)N1] | 2-(2,6-dioxopiperidin-3-yl)-5-((6-(4-(8- fluoroquinoxalin-2-yl)-1H-pyrazol-1- yl)hexyl)amino)isoindoline-1,3-dione |
| GS-378 | 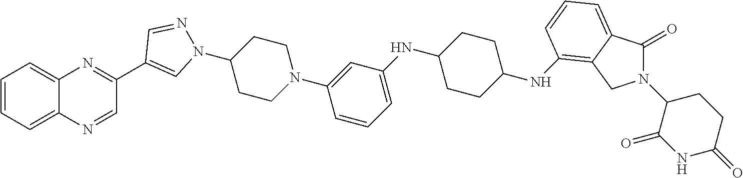 [SMILES: O=C1CCC(N2Cc3c(NC4CCC(Nc5cccc(N6CCC(n7cc(-c8cnc9ccccc9n8)cn7)CC6)c5)CC4)cccc3C2=O)C(=O)N1] | 3-(1-oxo-4-((4-((3-(4-(4-(quinoxalin-2- yl)-1H-pyrazol-1-yl)piperidin-1- yl)phenyl)amino)cyclohexyl)amino)iso- indolin-2-yl)piperidine-2,6-dione |
| GS-379 | 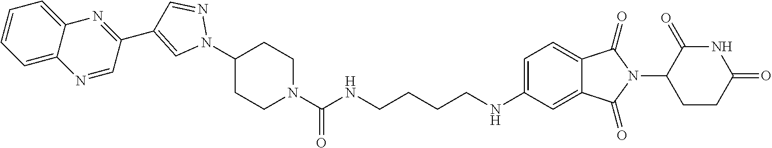 [SMILES: O=C1CCC(N2C(=O)c3ccc(NCCCCNC(=O)N4CCC(n5cc(-c6cnc7ccccc7n6)cn5)CC4)cc3C2=O)C(=O)N1] | N-(4-((2-(2,6-dioxopiperidin-3-yl)-1,3- dioxoisoindolin-5-yl)amino)butyl)-4-(4- (quinoxalin-2-yl)-1H-pyrazol-1- yl)piperidine-1-carboxamide |
| GS-380 | 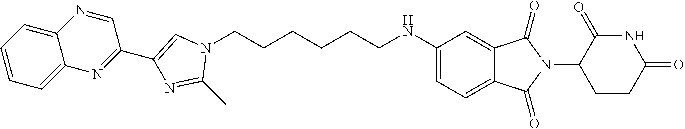 [SMILES: Cc1nc(-c2cnc3ccccc3n2)cn1CCCCCCNc1ccc2c(c1)C(=O)N(C1CCC(=O)NC1=O)C2=O] | 2-(2,6-dioxopiperidin-3-yl)-5-((6-(2- methyl-4-(quinoxalin-2-yl)-1H-imidazol- 1-yl)hexyl)amino)isoindoline-1,3-dione |
| GS-381 | 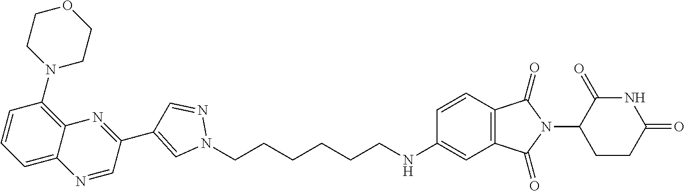 [SMILES: O=C1CCC(N2C(=O)c3ccc(NCCCCCCn4cc(-c5cnc6cccc(N7CCOCC7)c6n5)cn4)cc3C2=O)C(=O)N1] | 2-(2,6-dioxopiperidin-3-yl)-5-((6-(4-(8- morpholinoquinoxalin-2-yl)-1H-pyrazol- 1-yl)hexyl)amino)isoindoline-1,3-dione |
| GS-382 | 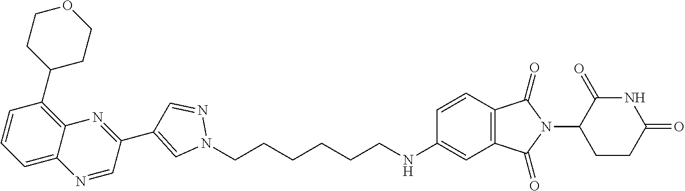 [SMILES: O=C1CCC(N2C(=O)c3ccc(NCCCCCCn4cc(-c5cnc6cccc(C7CCOCC7)c6n5)cn4)cc3C2=O)C(=O)N1] | 2-(2,6-dioxopiperidin-3-yl)-5-((6-(4-(8- (tetrahydro-2H-pyran-4-yl)quinoxalin-2- yl)-1H-pyrazol-1- yl)hexyl)amino)isoindoline-1,3-dione |
| GS-383 | 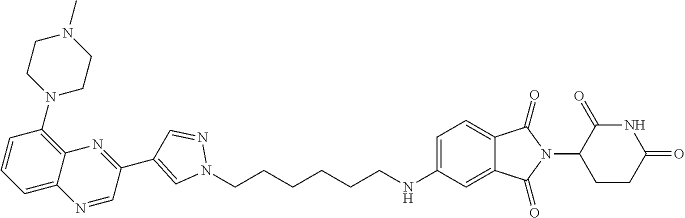 [SMILES: CN1CCN(c2cccc3ncc(-c4cnn(CCCCCCNc5ccc6c(c5)C(=O)N(C5CCC(=O)NC5=O)C6=O)c4)nc23)CC1] | 2-(2,6-dioxopiperidin-3-yl)-5-((6-(4-(8- (4-methylpiperazin-1-yl)quinoxalin-2-yl)- 1H-pyrazol-1- yl)hexyl)amino)isoindoline-1,3-dione |
| GS-384 | 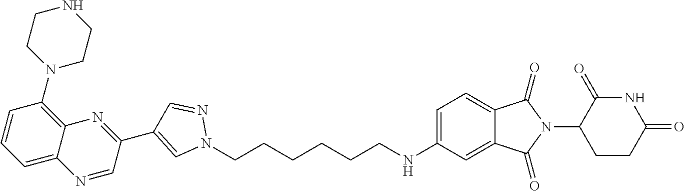 [SMILES: O=C1CCC(N2C(=O)c3ccc(NCCCCCCn4cc(-c5cnc6cccc(N7CCNCC7)c6n5)cn4)cc3C2=O)C(=O)N1] | 2-(2,6-dioxopiperidin-3-yl)-5-((6-(4-(8- (piperazin-1-yl)quinoxalin-2-yl)-1H- pyrazol-1-yl)hexyl)amino)isoindoline- 1,3-dione |
| GS-385 | 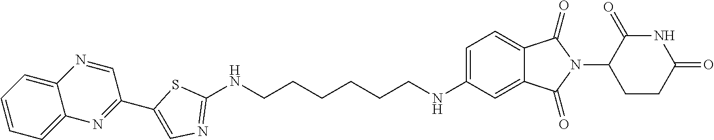 [SMILES: O=C1CCC(N2C(=O)c3ccc(NCCCCCCNc4ncc(-c5cnc6ccccc6n5)s4)cc3C2=O)C(=O)N1] | 2-(2,6-dioxopiperidin-3-yl)-5-((6-((5- (quinoxalin-2-yl)thiazol-2- yl)amino)hexyl)amino)isoindoline-1,3- dione |
| GS-386 | 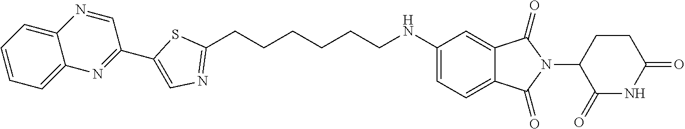 [SMILES: O=C1CCC(N2C(=O)c3ccc(NCCCCCCc4ncc(-c5cnc6ccccc6n5)s4)cc3C2=O)C(=O)N1] | 2-(2,6-dioxopiperidin-3-yl)-5-((6-(5- (quinoxalin-2-yl)thiazol-2- yl)hexyl)amino)isoindoline-1,3-dione |
| GS-387 | 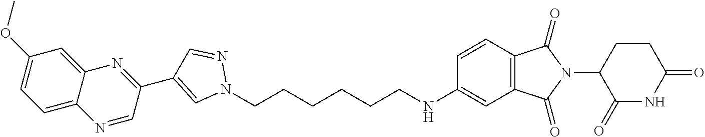 [SMILES: COc1ccc2ncc(-c3cnn(CCCCCCNc4ccc5c(c4)C(=O)N(C4CCC(=O)NC4=O)C5=O)c3)nc2c1] | 2-(2,6-dioxopiperidin-3-yl)-5-((6-(4-(7- methoxyquinoxalin-2-yl)-1H-pyrazol-1- yl)hexyl)amino)isoindoline-1,3-dione |
| GS-388 | 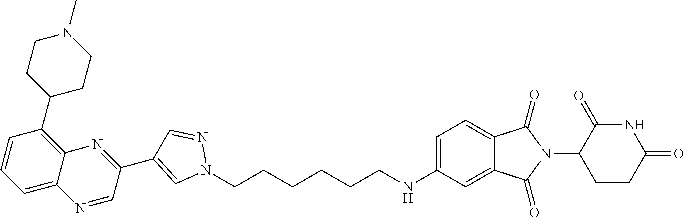 [SMILES: CN1CCC(c2cccc3ncc(-c4cnn(CCCCCCNc5ccc6c(c5)C(=O)N(C5CCC(=O)NC5=O)C6=O)c4)nc23)CC1] | 2-(2,6-dioxopiperidin-3-yl)-5-((6-(4-(8- (1-methylpiperidin-4-yl)quinoxalin-2-yl)- 1H-pyrazol-1- yl)hexyl)amino)isoindoline-1,3-dione |
| GS-389 | 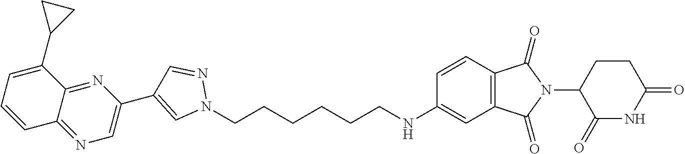 [SMILES: O=C1CCC(N2C(=O)c3ccc(NCCCCCCn4cc(-c5cnc6cccc(C7CC7)c6n5)cn4)cc3C2=O)C(=O)N1] | 5-((6-(4-(8-cyclopropylquinoxalin-2-yl)- 1H-pyrazol-1-yl)hexyl)amino)-2-(2,6- dioxopiperidin-3-yl)isoindoline-1,3-dione |
| GS-390 | 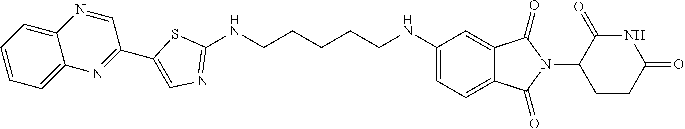 [SMILES: O=C1CCC(N2C(=O)c3ccc(NCCCCCNc4ncc(-c5cnc6ccccc6n5)s4)cc3C2=O)C(=O)N1] | 2-(2,6-dioxopiperidin-3-yl)-5-((5-((5- (quinoxalin-2-yl)thiazol-2- yl)amino)pentyl)amino)isoindoline-1,3- dione |
| GS-391 | 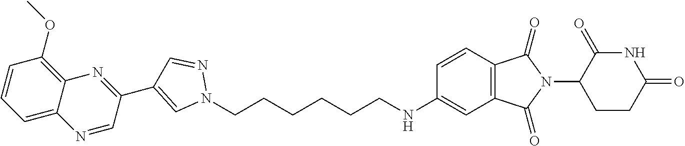 [SMILES: COc1cccc2ncc(-c3cnn(CCCCCCNc4ccc5c(c4)C(=O)N(C4CCC(=O)NC4=O)C5=O)c3)nc12] | 2-(2,6-dioxopiperidin-3-yl)-5-((6-(4-(8- methoxyquinoxalin-2-yl)-1H-pyrazol-1- yl)hexyl)amino)isoindoline-1,3-dione |
| GS-392 | 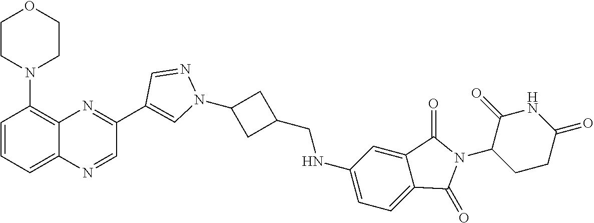 [SMILES: O=C1CCC(N2C(=O)c3ccc(NCC4CC(n5cc(-c6cnc7cccc(N8CCOCC8)c7n6)cn5)C4)cc3C2=O)C(=O)N1] | 2-(2,6-dioxopiperidin-3-yl)-5-((6-(4-(8- methoxyquinoxalin-2-yl)-1H-pyrazol-1- yl)hexyl)amino)isoindoline-1,3-dione |
| GS-393 | 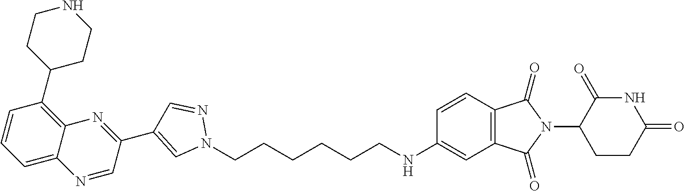 [SMILES: O=C1CCC(N2C(=O)c3ccc(NCCCCCCn4cc(-c5cnc6cccc(C7CCNCC7)c6n5)cn4)cc3C2=O)C(=O)N1] | 2-(2,6-dioxopiperidin-3-yl)-5-((6-(4-(8- (piperidin-4-yl)quinoxalin-2-yl)-1H- pyrazol-1-yl)hexyl)amino)isoindoline- 1,3-dione |
| GS-394 | 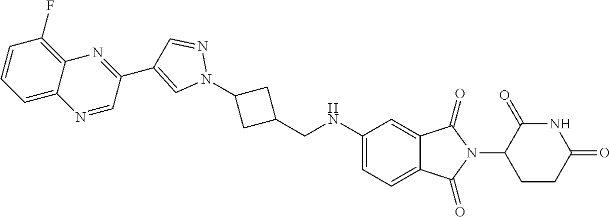 [SMILES: O=C1CCC(N2C(=O)c3ccc(NCC4CC(n5cc(-c6cnc7cccc(F)c7n6)cn5)C4)cc3C2=O)C(=O)N1] | 2-(2,6-dioxopiperidin-3-yl)-5-(((3-(4-(8- fluoroquinoxalin-2-yl)-1H-pyrazol-1- yl)cyclobutyl)methyl)amino)isoindoline- 1,3-dione |
| GS-395 | 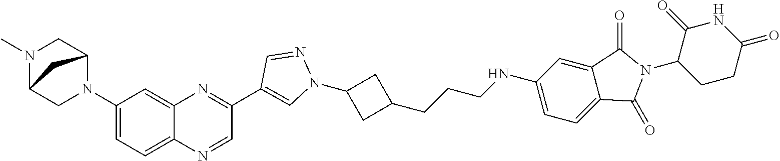 [SMILES: CN1C[C@H]2C[C@@H]1CN2c1ccc2ncc(-c3cnn(C4CC(CCCNc5ccc6c(c5)C(=O)N(C5CCC(=O)NC5=O)C6=O)C4)c3)nc2c1] | 2-(2,6-dioxopiperidin-3-yl)-5-((3-(3-(4- (7-((1R,4R)-5-methyl-2,5- diazabicyclo[2.2.1]heptan-2- yl)quinoxalin-2-yl)-1H-pyrazol-1- yl)cyclobutyl)propyl)amino)isoindoline- 1,3-dione |
| GS-396 | 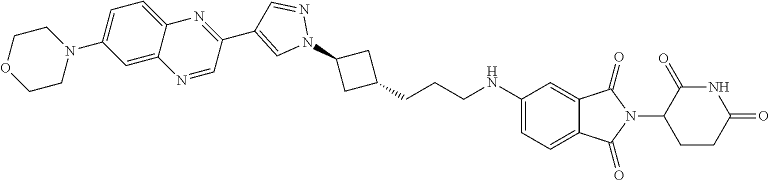 [SMILES: O=C1CCC(N2C(=O)c3ccc(NCCC[C@H]4C[C@H](n5cc(-c6cnc7cc(N8CCOCC8)ccc7n6)cn5)C4)cc3C2=O)C(=O)N1] | 2-(2,6-dioxopiperidin-3-yl)-5-((3-(trans- 3-(4-(6-morpholinoquinoxalin-2-yl)-1H- pyrazol-1- yl)cyclobutyl)propyl)amino)isoindoline- 1,3-dione |
| GS-397 | 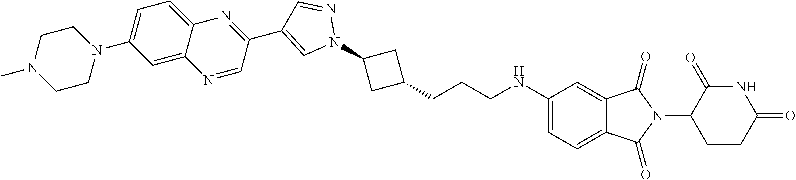 [SMILES: CN1CCN(c2ccc3nc(-c4cnn([C@H]5C[C@H](CCCNc6ccc7c(c6)C(=O)N(C6CCC(=O)NC6=O)C7=O)C5)c4)cnc3c2)CC1] | 2-(2,6-dioxopiperidin-3-yl)-5-((3-(trans- 3-(4-(6-(4-methylpiperazin-1- yl)quinoxalin-2-yl)-1H-pyrazol-1- yl)cyclobutyl)propyl)amino)isoindoline- 1,3-dione |
| GS-398 | 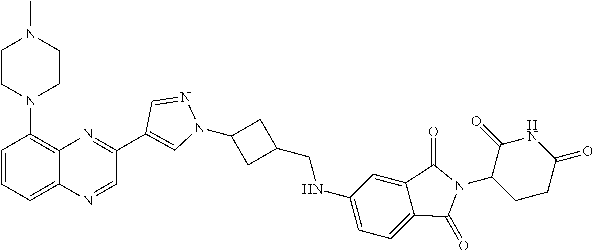 [SMILES: CN1CCN(c2cccc3ncc(-c4cnn(C5CC(CNc6ccc7c(c6)C(=O)N(C6CCC(=O)NC6=O)C7=O)C5)c4)nc23)CC1] | 2-(2,6-dioxopiperidin-3-yl)-5-(((3-(4-(8- (4-methylpiperazin-1-yl)quinoxalin-2-yl)- 1H-pyrazol-1- yl)cyclobutyl)methyl)amino)isoindoline- 1,3-dione |
| GS-399 | 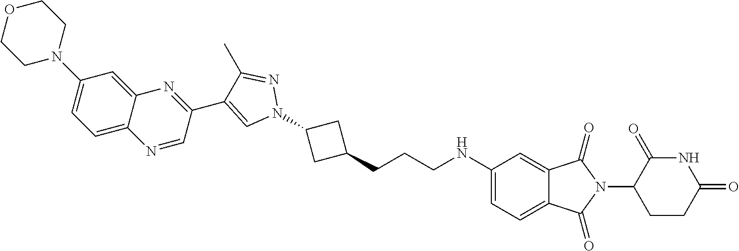 [SMILES: Cc1nn([C@H]2C[C@H](CCCNc3ccc4c(c3)C(=O)N(C3CCC(=O)NC3=O)C4=O)C2)cc1-c1cnc2ccc(N3CCOCC3)cc2n1] | 2-(2,6-dioxopiperidin-3-yl)-5-((3-(trans- 3-(3-methyl-4-(7-morpholinoquinoxalin- 2-yl)-1H-pyrazol-1- yl)cyclobutyl)propyl)amino)isoindoline- 1,3-dione |
| GS-400 | 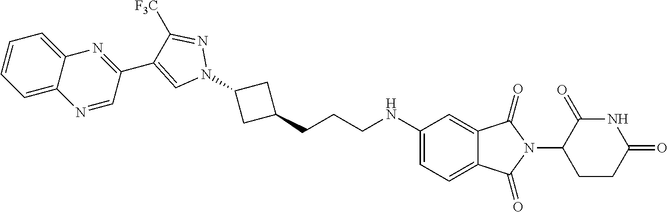 [SMILES: O=C1CCC(N2C(=O)c3ccc(NCCC[C@H]4C[C@H](n5cc(-c6cnc7ccccc7n6)c(C(F)(F)F)n5)C4)cc3C2=O)C(=O)N1] | 2-(2,6-dioxopiperidin-3-yl)-5-((3-(trans- 3-(4-(quinoxalin-2-yl)-3- (trifluoromethyl)-1H-pyrazol-1- yl)cyclobutyl)propyl)amino)isoindoline- 1,3-dione |
| GS-401 | 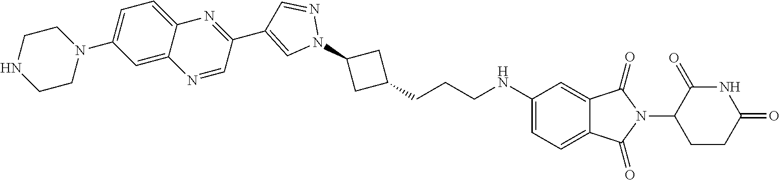 [SMILES: O=C1CCC(N2C(=O)c3ccc(NCCC[C@H]4C[C@H](n5cc(-c6cnc7cc(N8CCNCC8)ccc7n6)cn5)C4)cc3C2=O)C(=O)N1] | 2-(2,6-dioxopiperidin-3-yl)-5-((3-(trans- 3-(4-(6-(piperazin-1-yl)quinoxalin-2-yl)- 1H-pyrazol-1- yl)cyclobutyl)propyl)amino)isoindoline- 1,3-dione |
| GS-402 | 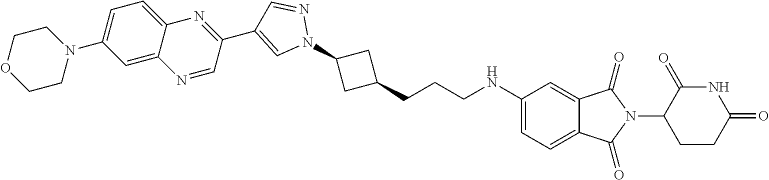 [SMILES: O=C1CCC(N2C(=O)c3ccc(NCCC[C@H]4C[C@@H](n5cc(-c6cnc7cc(N8CCOCC8)ccc7n6)cn5)C4)cc3C2=O)C(=O)N1] | 2-(2,6-dioxopiperidin-3-yl)-5-((3-(cis-3- (4-(6-morpholinoquinoxalin-2-yl)-1H- pyrazol-1- 1280yl)cyclobutyl)propyl)amino)isoindoline- 1,3-dione |
| GS-403 | 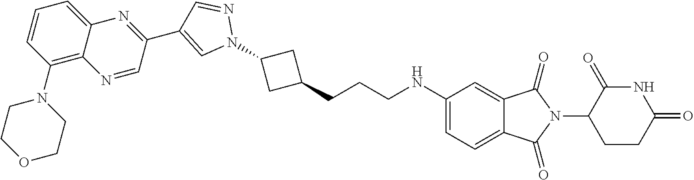 [SMILES: O=C1CCC(N2C(=O)c3ccc(NCCC[C@H]4C[C@H](n5cc(-c6cnc7c(N8CCOCC8)cccc7n6)cn5)C4)cc3C2=O)C(=O)N1] | 2-(2,6-dioxopiperidin-3-yl)-5-((3-(trans- 3-(4-(5-morpholinoquinoxalin-2-yl)-1H- pyrazol-1- yl)cyclobutyl)propyl)amino)isoindoline- 1,3-dione |
| GS-404 | 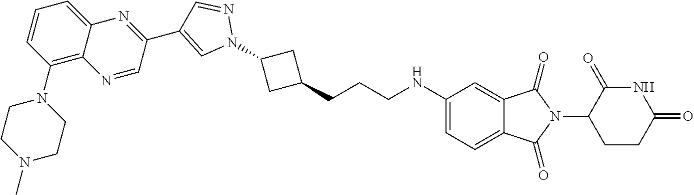 [SMILES: CN1CCN(c2cccc3nc(-c4cnn([C@H]5C[C@H](CCCNc6ccc7c(c6)C(=O)N(C6CCC(=O)NC6=O)C7=O)C5)c4)cnc23)CC1] | 2-(2,6-dioxopiperidin-3-yl)-5-((3-(trans- 3-(4-(5-(4-methylpiperazin-1- yl)quinoxalin-2-yl)-1H-pyrazol-1- yl)cyclobutyl)propyl)amino)isoindoline- 1,3-dione |
| GS-405 | 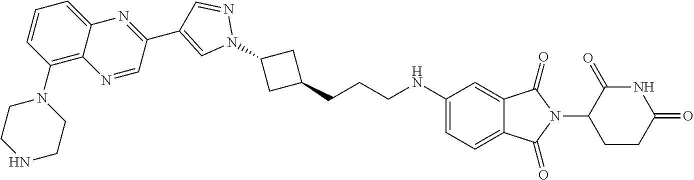 [SMILES: O=C1CCC(N2C(=O)c3ccc(NCCC[C@H]4C[C@H](n5cc(-c6cnc7c(N8CCNCC8)cccc7n6)cn5)C4)cc3C2=O)C(=O)N1] | 2-(2,6-dioxopiperidin-3-yl)-5-((3-(trans- 3-(4-(5-(piperazin-1-yl)quinoxalin-2-yl)- 1H-pyrazol-1- yl)cyclobutyl)propyl)amino)isoindoline- 1,3-dione |
| GS-406 | 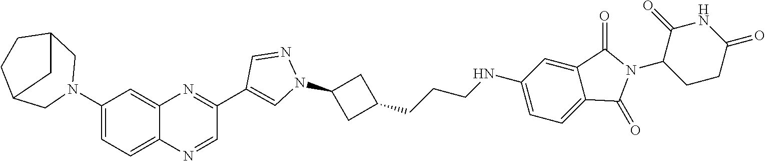 [SMILES: O=C1CCC(N2C(=O)c3ccc(NCCC[C@H]4C[C@H](n5cc(-c6cnc7ccc(N8CC9CCC(C9)C8)cc7n6)cn5)C4)cc3C2=O)C(=O)N1] | 5-((3-(trans-3-(4-(7-(8-oxa-3- azabicyclo[3.2.1]octan-3-yl)quinoxalin-2- yl)-1H-pyrazol-1- yl)cyclobutyl)propyl)amino)-2-(2,6- dioxopiperidin-3-yl)isoindoline-1,3-dione |
| GS-407 | 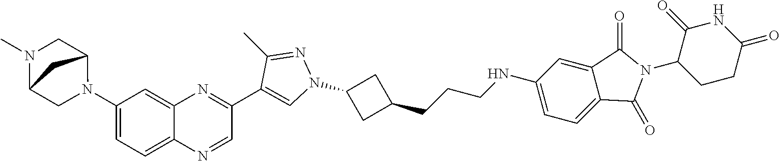 [SMILES: Cc1nn([C@H]2C[C@H](CCCNc3ccc4c(c3)C(=O)N(C3CCC(=O)NC3=O)C4=O)C2)cc1-c1cnc2ccc(N3C[C@H]4C[C@@H]3CN4C)cc2n1] | 2-(2,6-dioxopiperidin-3-yl)-5-((3-(trans- 3-(3-methyl-4-(7-((1R,4R)-5-methyl-2,5- diazabicyclo[2.2.1]heptan-2- yl)quinoxalin-2-yl)-1H-pyrazol-1- yl)cyclobutyl)propyl)amino)isoindoline- 1,3-dione |
| GS-408 | 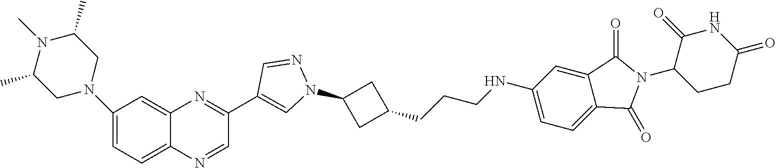 [SMILES: C[C@@H]1CN(c2ccc3ncc(-c4cnn([C@H]5C[C@H](CCCNc6ccc7c(c6)C(=O)N(C6CCC(=O)NC6=O)C7=O)C5)c4)nc3c2)C[C@H](C)N1C] | 2-(2,6-dioxopiperidin-3-yl)-5-((3-(trans- 3-(4-(7-((3S,5R)-3,4,5- trimethylpiperazin-1-yl)quinoxalin-2-yl)- 1H-pyrazol-1- yl)cyclobutyl)propyl)amino)isoindoline- 1,3-dione |
| GS-409 | 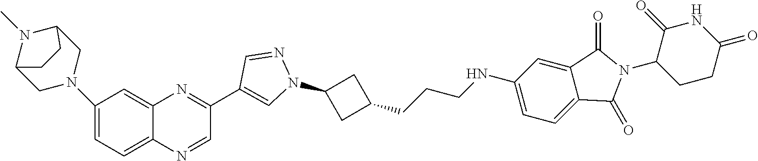 [SMILES: CN1C2CCC1CN(c1ccc3ncc(-c4cnn([C@H]5C[C@H](CCCNc6ccc7c(c6)C(=O)N(C6CCC(=O)NC6=O)C7=O)C5)c4)nc3c1)C2] | 2-(2,6-dioxopiperidin-3-yl)-5-((3-(trans- 3-(4-(7-(8-methyl-3,8- diazabicyclo[3.2.1]octan-3-yl)quinoxalin- 2-yl)-1H-pyrazol-1- yl)cyclobutyl)propyl)amino)isoindoline- 1,3-dione |
| GS-410 | 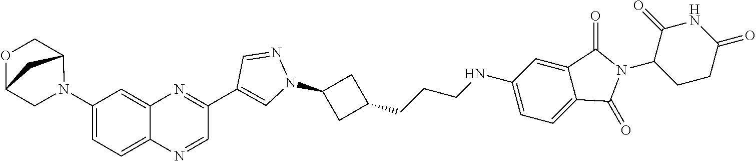 [SMILES: O=C1CCC(N2C(=O)c3ccc(NCCC[C@H]4C[C@H](n5cc(-c6cnc7ccc(N8C[C@H]9C[C@@H]8CO9)cc7n6)cn5)C4)cc3C2=O)C(=O)N1] | 5-((3-(trans-3-(4-(7-((1R,4R)-2-oxa-5- azabicyclo[2.2.1]heptan-5-yl)quinoxalin- 2-yl)-1H-pyrazol-1- yl)cyclobutyl)propyl)amino)-2-(2,6- dioxopiperidin-3-yl)isoindoline-1,3-dione |
| GS-411 | 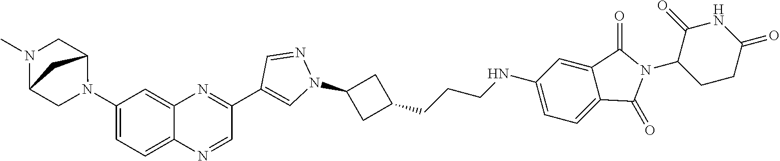 [SMILES: CN1C[C@H]2C[C@@H]1CN2c1ccc2ncc(-c3cnn([C@H]4C[C@H](CCCNc5ccc6c(c5)C(=O)N(C5CCC(=O)NC5=O)C6=O)C4)c3)nc2c1] | 2-(2,6-dioxopiperidin-3-yl)-5-((3-(trans- 3-(4-(7-((1R,4R)-5-methyl-2,5- diazabicyclo[2.2.1]heptan-2- yl)quinoxalin-2-yl)-1H-pyrazol-1- yl)cyclobutyl)propyl)amino)isoindoline- 1,3-dione |
| GS-412 | 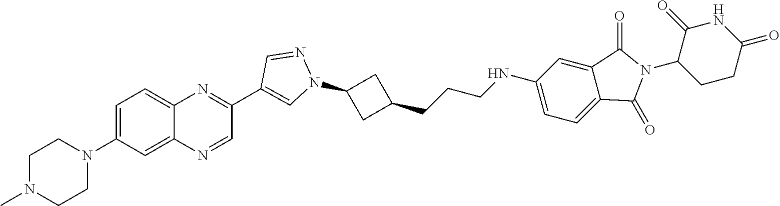 [SMILES: CN1CCN(c2ccc3nc(-c4cnn([C@H]5C[C@@H](CCCNc6ccc7c(c6)C(=O)N(C6CCC(=O)NC6=O)C7=O)C5)c4)cnc3c2)CC1] | 2-(2,6-dioxopiperidin-3-yl)-5-((3-(cis-3- (4-(6-(4-methylpiperazin-1- yl)quinoxalin-2-yl)-1H-pyrazol-1- yl)cyclobutyl)propyl)amino)isoindoline- 1,3-dione |
| GS-413 | 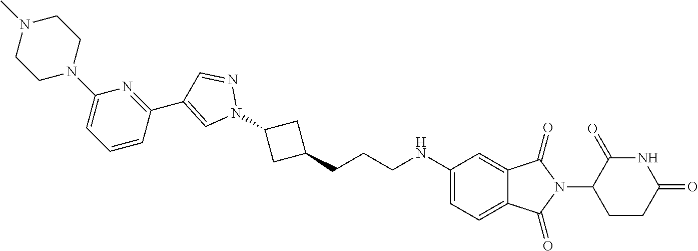 [SMILES: CN1CCN(c2cccc(-c3cnn([C@H]4C[C@H](CCCNc5ccc6c(c5)C(=O)N(C5CCC(=O)NC5=O)C6=O)C4)c3)n2)CC1] | 2-(2,6-dioxopiperidin-3-yl)-5-((3-(trans- 3-(4-(6-(4-methylpiperazin-1-yl)pyridin- 2-yl)-1H-pyrazol-1- yl)cyclobutyl)propyl)amino)isoindoline- 1,3-dione |
| GS-414 | 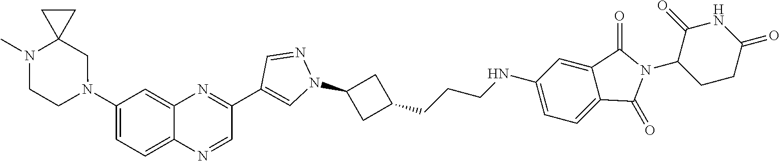 [SMILES: CN1CCN(c2ccc3ncc(-c4cnn([C@H]5C[C@H](CCCNc6ccc7c(c6)C(=O)N(C6CCC(=O)NC6=O)C7=O)C5)c4)nc3c2)CC12CC2] | 2-(2,6-dioxopiperidin-3-yl)-5-((3-(trans- 3-(4-(7-(4-methyl-4,7- diazaspiro[2.5]octan-7-yl)quinoxalin-2- yl)-1H-pyrazol-1- yl)cyclobutyl)propyl)amino)isoindoline- 1,3-dione |
| GS-415 | 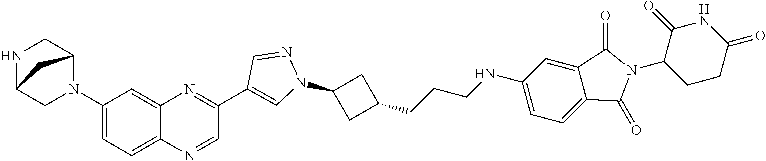 [SMILES: O=C1CCC(N2C(=O)c3ccc(NCCC[C@H]4C[C@H](n5cc(-c6cnc7ccc(N8C[C@H]9C[C@@H]8CN9)cc7n6)cn5)C4)cc3C2=O)C(=O)N1] | 5-((3-(trans-3-(4-(7-((1R,4R)-2,5- diazabicyclo[2.2.1]heptan-2- yl)quinoxalin-2-yl)-1H-pyrazol-1- yl)cyclobutyl)propyl)amino)-2-(2,6- dioxopiperidin-3-yl)isoindoline-1,3-dione |
| GS-416 | 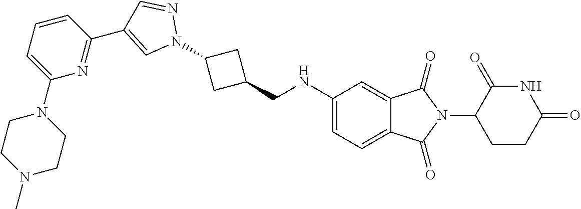 [SMILES: CN1CCN(c2cccc(-c3cnn([C@H]4C[C@H](CNc5ccc6c(c5)C(=O)N(C5CCC(=O)NC5=O)C6=O)C4)c3)n2)CC1] | 2-(2,6-dioxopiperidin-3-yl)-5-(((trans-3- (4-(6-(4-methylpiperazin-1-yl)pyridin-2- yl)-1H-pyrazol-1- yl)cyclobutyl)methyl)amino)isoindoline- 1,3-dione |
| GS-417 | 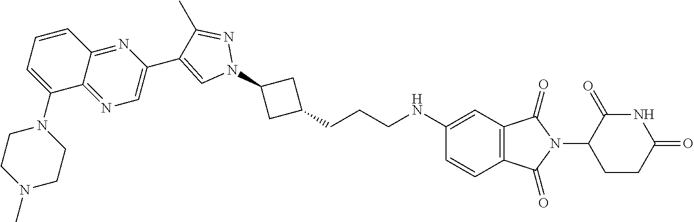 [SMILES: Cc1nn([C@H]2C[C@H](CCCNc3ccc4c(c3)C(=O)N(C3CCC(=O)NC3=O)C4=O)C2)cc1-c1cnc2c(N3CCN(C)CC3)cccc2n1] | 2-(2,6-dioxopiperidin-3-yl)-5-((3-(trans- 3-(3-methyl-4-(5-(4-methylpiperazin-1- yl)quinoxalin-2-yl)-1H-pyrazol-1- yl)cyclobutyl)propyl)amino)isoindoline- 1,3-dione |
| GS-418 | 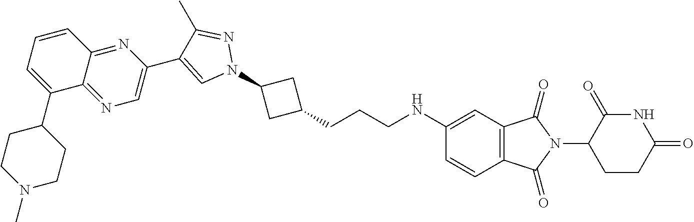 [SMILES: Cc1nn([C@H]2C[C@H](CCCNc3ccc4c(c3)C(=O)N(C3CCC(=O)NC3=O)C4=O)C2)cc1-c1cnc2c(C3CCN(C)CC3)cccc2n1] | 2-(2,6-dioxopiperidin-3-yl)-5-((3-(trans- 3-(3-methyl-4-(5-(1-methylpiperidin-4- yl)quinoxalin-2-yl)-1H-pyrazol-1- yl)cyclobutyl)propyl)amino)isoindoline- 1,3-dione |
| GS-419 | 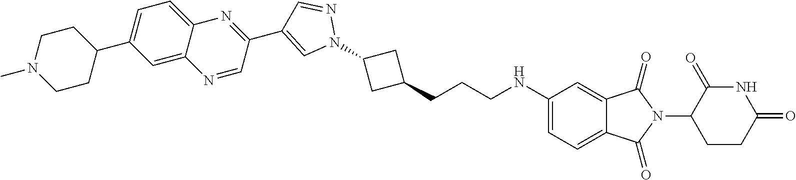 [SMILES: CN1CCC(c2ccc3nc(-c4cnn([C@H]5C[C@H](CCCNc6ccc7c(c6)C(=O)N(C6CCC(=O)NC6=O)C7=O)C5)c4)cnc3c2)CC1] | 2-(2,6-dioxopiperidin-3-yl)-5-((3-(trans- 3-(4-(6-(1-methylpiperidin-4- yl)quinoxalin-2-yl)-1H-pyrazol-1- yl)cyclobutyl)propyl)amino)isoindoline- 1,3-dione |
| GS-420 | 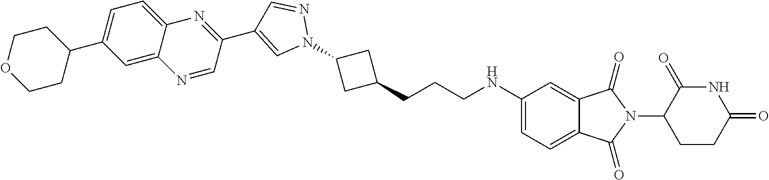 [SMILES: O=C1CCC(N2C(=O)c3ccc(NCCC[C@H]4C[C@H](n5cc(-c6cnc7cc(C8CCOCC8)ccc7n6)cn5)C4)cc3C2=O)C(=O)N1] | 2-(2,6-dioxopiperidin-3-yl)-5-((3-(trans- 3-(4-(6-(tetrahydro-2H-pyran-4- yl)quinoxalin-2-yl)-1H-pyrazol-1- yl)cyclobutyl)propyl)amino)isoindoline- 1,3-dione |
| GS-421 | 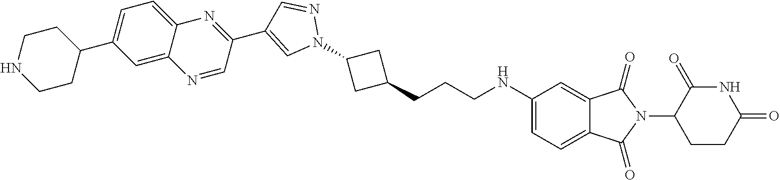 [SMILES: O=C1CCC(N2C(=O)c3ccc(NCCC[C@H]4C[C@H](n5cc(-c6cnc7cc(C8CCNCC8)ccc7n6)cn5)C4)cc3C2=O)C(=O)N1] | 2-(2,6-dioxopiperidin-3-yl)-5-((3-(trans- 3-(4-(6-(piperidin-4-yl)quinoxalin-2-yl)- 1H-pyrazol-1- yl)cyclobutyl)propyl)amino)isoindoline- 1,3-dione |
| GS-422 | 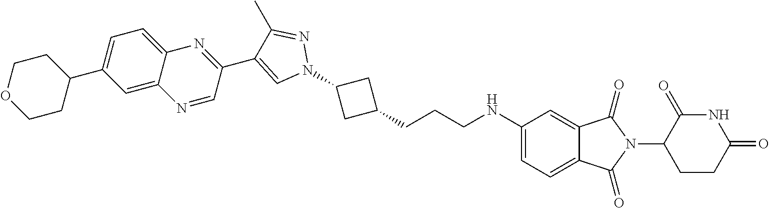 [SMILES: Cc1nn([C@H]2C[C@@H](CCCNc3ccc4c(c3)C(=O)N(C3CCC(=O)NC3=O)C4=O)C2)cc1-c1cnc2cc(C3CCOCC3)ccc2n1] | 2-(2,6-dioxopiperidin-3-yl)-5-((3-(cis-3- (3-methyl-4-(6-morpholinoquinoxalin-2- yl)-1H-pyrazol-1- yl)cyclobutyl)propyl)amino)isoindoline- 1,3-dione |
| GS-423 | 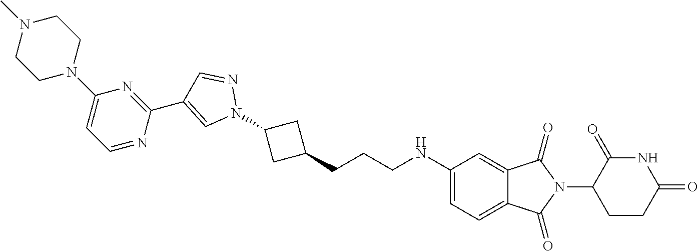 [SMILES: CN1CCN(c2ccnc(-c3cnn([C@H]4C[C@H](CCCNc5ccc6c(c5)C(=O)N(C5CCC(=O)NC5=O)C6=O)C4)c3)n2)CC1] | 2-(2,6-dioxopiperidin-3-yl)-5-((3-(trans- 3-(4-(4-(4-methylpiperazin-1- yl)pyrimidin-2-yl)-1H-pyrazol-1- yl)cyclobutyl)propyl)amino)isoindoline- 1,3-dione |
| GS-424 | 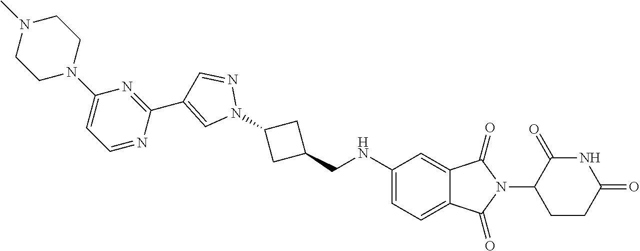 [SMILES: CN1CCN(c2ccnc(-c3cnn([C@H]4C[C@H](CNc5ccc6c(c5)C(=O)N(C5CCC(=O)NC5=O)C6=O)C4)c3)n2)CC1] | 2-(2,6-dioxopiperidin-3-yl)-5-(((trans-3- (4-(4-(4-methylpiperazin-1-yl)pyrimidin- 2-yl)-1H-pyrazol-1- yl)cyclobutyl)methyl)amino)isoindoline- 1,3-dione |
| GS-425 | 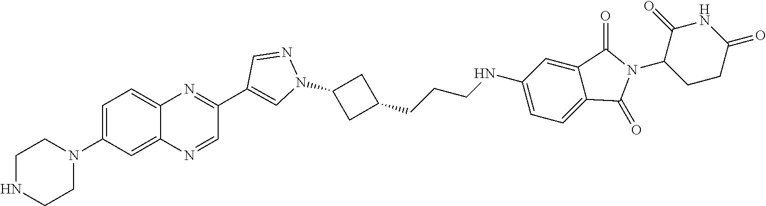 [SMILES: O=C1CCC(N2C(=O)c3ccc(NCCC[C@H]4C[C@@H](n5cc(-c6cnc7cc(N8CCNCC8)ccc7n6)cn5)C4)cc3C2=O)C(=O)N1] | 2-(2,6-dioxopiperidin-3-yl)-5-((3-(cis-3- (4-(6-(piperazin-1-yl)quinoxalin-2-yl)- 1H-pyrazol-1- yl)cyclobutyl)propyl)amino)isoindoline- 1,3-dione |
| GS-426 | 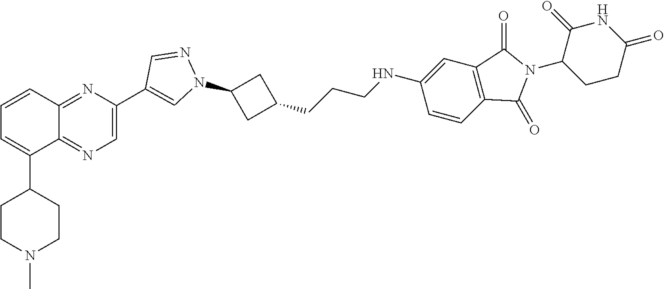 [SMILES: CN1CCC(c2cccc3nc(-c4cnn([C@H]5C[C@H](CCCNc6ccc7c(c6)C(=O)N(C6CCC(=O)NC6=O)C7=O)C5)c4)cnc23)CC1] | 2-(2,6-dioxopiperidin-3-yl)-5-((3-(trans- 3-(4-(5-(1-methylpiperidin-4- yl)quinoxalin-2-yl)-1H-pyrazol-1- yl)cyclobutyl)propyl)amino)isoindoline- 1,3-dione |
| GS-427 | 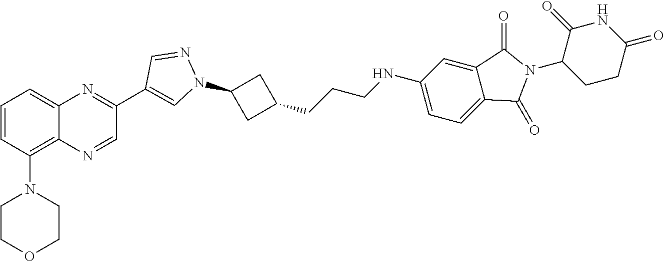 [SMILES: O=C1CCC(N2C(=O)c3ccc(NCCC[C@H]4C[C@H](n5cc(-c6cnc7c(N8CCOCC8)cccc7n6)cn5)C4)cc3C2=O)C(=O)N1] | 2-(2,6-dioxopiperidin-3-yl)-5-((3-(trans- 3-(4-(5-(tetrahydro-2H-pyran-4- yl)quinoxalin-2-yl)-1H-pyrazol-1- yl)cyclobutyl)propyl)amino)isoindoline- 1,3-dione |
| GS-428 | 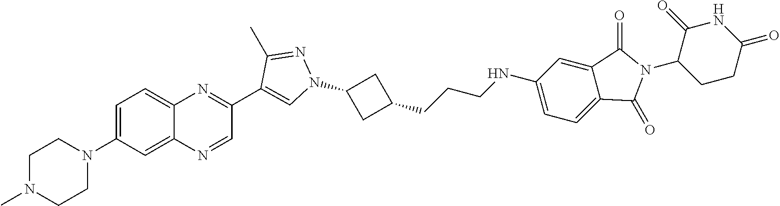 [SMILES: Cc1nn([C@H]2C[C@@H](CCCNc3ccc4c(c3)C(=O)N(C3CCC(=O)NC3=O)C4=O)C2)cc1-c1cnc2cc(N3CCN(C)CC3)ccc2n1] | 2-(2,6-dioxopiperidin-3-yl)-5-((3-(cis-3- (3-methyl-4-(6-(4-methylpiperazin-1- yl)quinoxalin-2-yl)-1H-pyrazol-1- yl)cyclobutyl)propyl)amino)isoindoline- 1,3-dione |
| GS-429 | 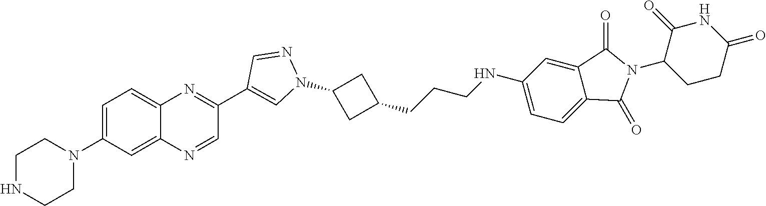 [SMILES: O=C1CCC(N2C(=O)c3ccc(NCCC[C@H]4C[C@@H](n5cc(-c6cnc7cc(N8CCNCC8)ccc7n6)cn5)C4)cc3C2=O)C(=O)N1] | 2-(2,6-dioxopiperidin-3-yl)-5-((3-(cis-3- (4-(6-(piperidin-4-yl)quinoxalin-2-yl)- 1H-pyrazol-1- yl)cyclobutyl)propyl)amino)isoindoline- 1,3-dione |
| GS-430 | 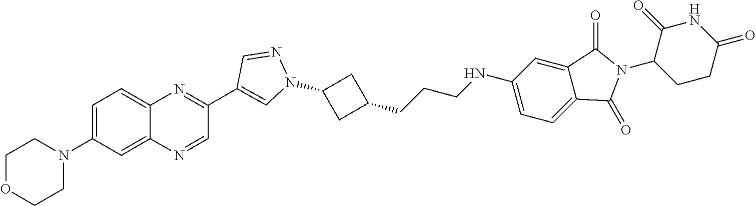 [SMILES: O=C1CCC(N2C(=O)c3ccc(NCCC[C@H]4C[C@@H](n5cc(-c6cnc7cc(N8CCOCC8)ccc7n6)cn5)C4)cc3C2=O)C(=O)N1] | 2-(2,6-dioxopiperidin-3-yl)-5-((3-(cis-3- (4-(6-(tetrahydro-2H-pyran-4- yl)quinoxalin-2-yl)-1H-pyrazol-1- yl)cyclobutyl)propyl)amino)isoindoline- 1,3-dione |
| GS-431 | 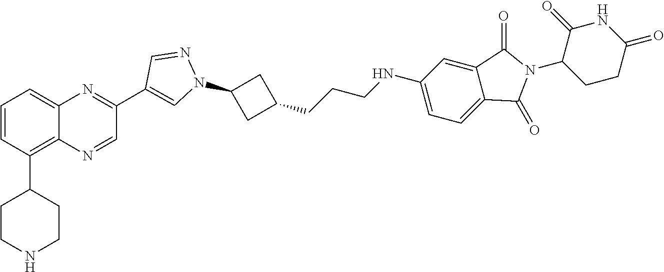 [SMILES: O=C1CCC(N2C(=O)c3ccc(NCCC[C@H]4C[C@H](n5cc(-c6cnc7c(C8CCNCC8)cccc7n6)cn5)C4)cc3C2=O)C(=O)N1] | 2-(2,6-dioxopiperidin-3-yl)-5-((3-(trans- 3-(4-(5-(piperidin-4-yl)quinoxalin-2-yl)- 1H-pyrazol-1- yl)cyclobutyl)propyl)amino)isoindoline- 1,3-dione |
| GS-432 | 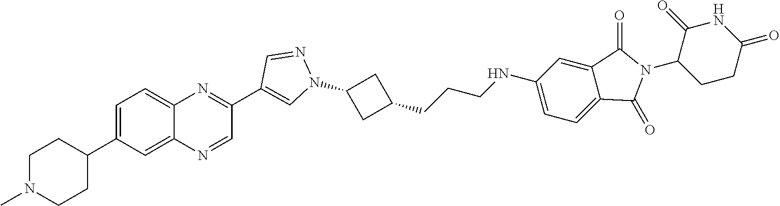 [SMILES: CN1CCC(c2ccc3nc(-c4cnn([C@H]5C[C@@H](CCCNc6ccc7c(c6)C(=O)N(C6CCC(=O)NC6=O)C7=O)C5)c4)cnc3c2)CC1] | 2-(2,6-dioxopiperidin-3-yl)-5-((3-(cis-3- (4-(6-(1-methylpiperidin-4-yl)quinoxalin- 2-yl)-1H-pyrazol-1- yl)cyclobutyl)propyl)amino)isoindoline- 1,3-dione |
| GS-433 | 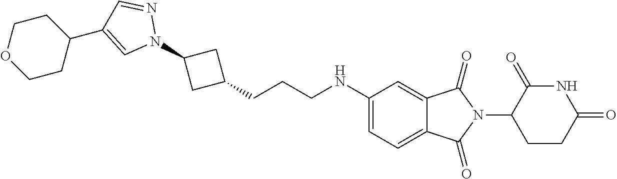 [SMILES: O=C1CCC(N2C(=O)c3ccc(NCCC[C@H]4C[C@H](n5cc(C6CCOCC6)cn5)C4)cc3C2=O)C(=O)N1] | 2-(2,6-dioxopiperidin-3-yl)-5-((3-(trans- 3-(4-(tetrahydro-2H-pyran-4-yl)-1H- pyrazol-1- yl)cyclobutyl)propyl)amino)isoindoline- 1,3-dione |
| GS-434 | 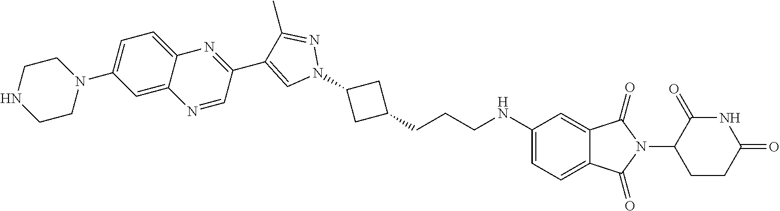 [SMILES: Cc1nn([C@H]2C[C@@H](CCCNc3ccc4c(c3)C(=O)N(C3CCC(=O)NC3=O)C4=O)C2)cc1-c1cnc2cc(N3CCNCC3)ccc2n1] | 2-(2,6-dioxopiperidin-3-yl)-5-((3-(cis-3- (3-methyl-4-(6-(piperazin-1- yl)quinoxalin-2-yl)-1H-pyrazol-1- yl)cyclobutyl)propyl)amino)isoindoline- 1,3-dione |
| GS-435 | 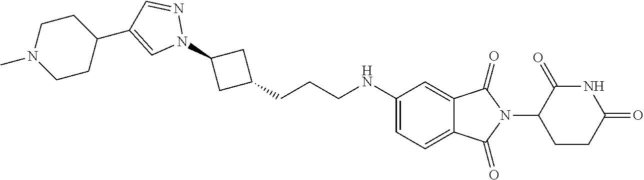 [SMILES: CN1CCC(c2cnn([C@H]3C[C@H](CCCNc4ccc5c(c4)C(=O)N(C4CCC(=O)NC4=O)C5=O)C3)c2)CC1] | 2-(2,6-dioxopiperidin-3-yl)-5-((3-(trans- 3-(4-(1-methylpiperidin-4-yl)-1H- pyrazol-1- yl)cyclobutyl)propyl)amino)isoindoline- 1,3-dione |
| GS-436 | 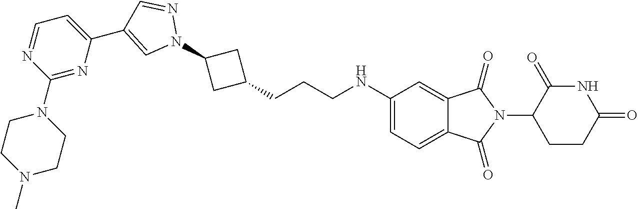 [SMILES: CN1CCN(c2nccc(-c3cnn([C@H]4C[C@H](CCCNc5ccc6c(c5)C(=O)N(C5CCC(=O)NC5=O)C6=O)C4)c3)n2)CC1] | 2-(2,6-dioxopiperidin-3-yl)-5-((3-(trans- 3-(4-(2-(4-methylpiperazin-1- yl)pyrimidin-4-yl)-1H-pyrazol-1- yl)cyclobutyl)propyl)amino)isoindoline- 1,3-dione |
| GS-437 | 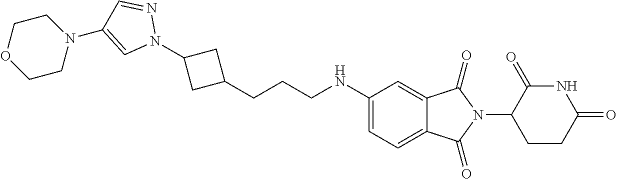 [SMILES: O=C1CCC(N2C(=O)c3ccc(NCCCC4CC(n5cc(N6CCOCC6)cn5)C4)cc3C2=O)C(=O)N1] | 2-(2,6-dioxopiperidin-3-yl)-5-((3-(3-(4- morpholino-1H-pyrazol-1- yl)cyclobutyl)propyl)amino)isoindoline- 1,3-dione |
| GS-438 | 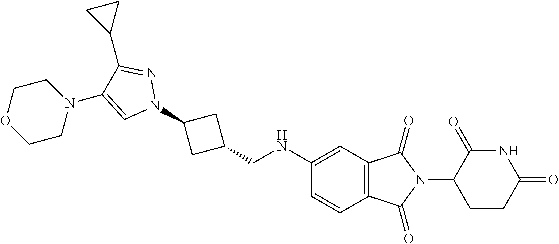 [SMILES: O=C1CCC(N2C(=O)c3ccc(NC[C@H]4C[C@H](n5cc(N6CCOCC6)c(C6CC6)n5)C4)cc3C2=O)C(=O)N1] | 5-(((trans-3-(3-cyclopropyl-4- morpholino-1H-pyrazol-1- yl)cyclobutyl)methyl)amino)-2-(2,6- dioxopiperidin-3-yl)isoindoline-1,3-dione |
| GS-439 | 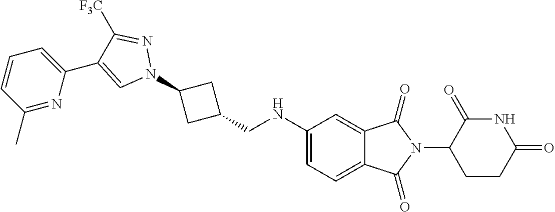 [SMILES: Cc1cccc(-c2cn([C@H]3C[C@H](CNc4ccc5c(c4)C(=O)N(C4CCC(=O)NC4=O)C5=O)C3)nc2C(F)(F)F)n1] | 2-(2,6-dioxopiperidin-3-yl)-5-(((trans-3- (4-(6-methylpyridin-2-yl)-3- (trifluoromethyl)-1H-pyrazol-1- yl)cyclobutyl)methyl)amino)isoindoline- 1,3-dione |
| GS-440 | 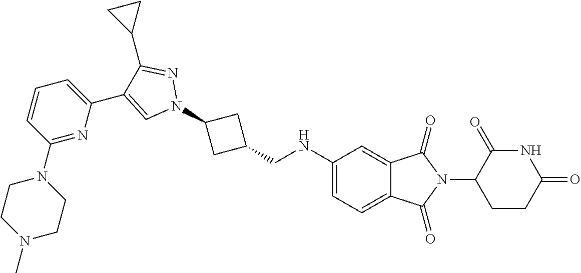 [SMILES: CN1CCN(c2cccc(-c3cn([C@H]4C[C@H](CNc5ccc6c(c5)C(=O)N(C5CCC(=O)NC5=O)C6=O)C4)nc3C3CC3)n2)CC1] | 5-(((trans-3-(3-cyclopropyl-4-(6-(4- methylpiperazin-1-yl)pyridin-2-yl)-1H- pyrazol-1-yl)cyclobutyl)methyl)amino)- 2-(2,6-dioxopiperidin-3-yl)isoindoline- 1,3-dione |
| GS-441 | 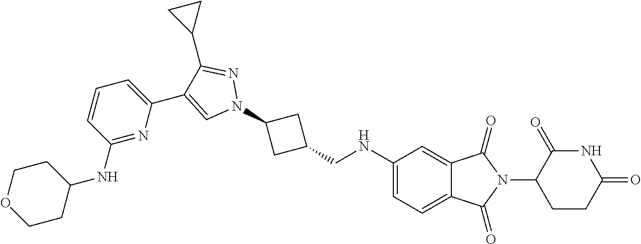 [SMILES: O=C1CCC(N2C(=O)c3ccc(NC[C@H]4C[C@H](n5cc(-c6cccc(NC7CCOCC7)n6)c(C6CC6)n5)C4)cc3C2=O)C(=O)N1] | 5-(((trans-3-(3-cyclopropyl-4-(6- ((tetrahydro-2H-pyran-4- yl)amino)pyridin-2-yl)-1H-pyrazol-1- yl)cyclobutyl)methyl)amino)-2-(2,6- dioxopiperidin-3-yl)isoindoline-1,3-dione |
| GS-442 | 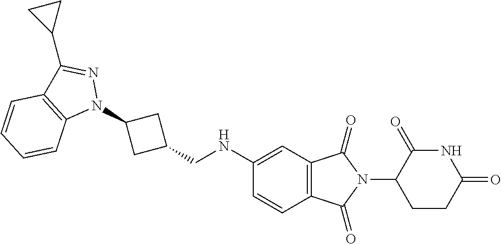 [SMILES: O=C1CCC(N2C(=O)c3ccc(NC[C@H]4C[C@H](n5nc(C6CC6)c6ccccc65)C4)cc3C2=O)C(=O)N1] | 5-(((trans-3-(3-cyclopropyl-1H-indazol- 1-yl)cyclobutyl)methyl)amino)-2-(2,6- dioxopiperidin-3-yl)isoindoline-1,3-dione |
| GS-443 | 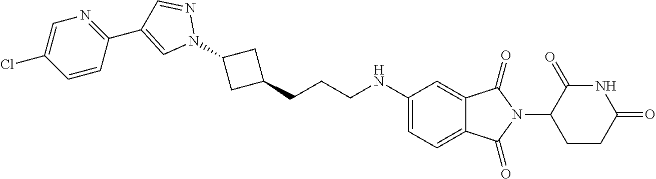 [SMILES: O=C1CCC(N2C(=O)c3ccc(NCCC[C@H]4C[C@H](n5cc(-c6ccc(Cl)cn6)cn5)C4)cc3C2=O)C(=O)N1] | 5-((3-(trans-3-(4-(5-chloropyridin-2-yl)- 1H-pyrazol-1- yl)cyclobutyl)propyl)amino)-2-(2,6- dioxopiperidin-3-yl)isoindoline-1,3-dione |
| GS-444 | 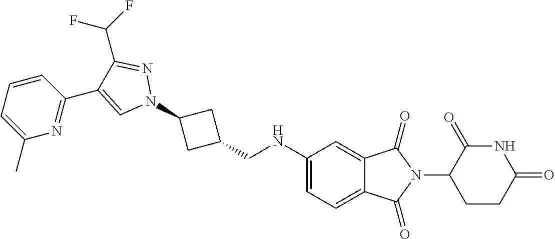 [SMILES: Cc1cccc(-c2cn([C@H]3C[C@H](CNc4ccc5c(c4)C(=O)N(C4CCC(=O)NC4=O)C5=O)C3)nc2C(F)F)n1] | 5-(((trans-3-(3-(difluoromethyl)-4-(6- methylpyridin-2-yl)-1H-pyrazol-1- yl)cyclobutyl)methyl)amino)-2-(2,6- dioxopiperidin-3-yl)isoindoline-1,3-dione |
| GS-445 | 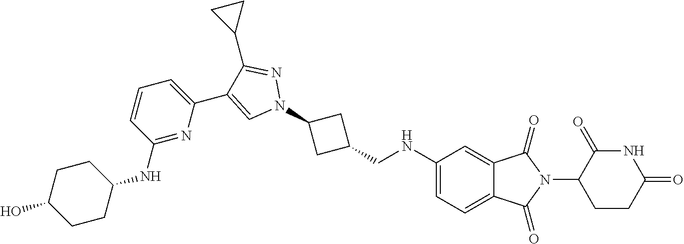 [SMILES: O=C1CCC(N2C(=O)c3ccc(NC[C@H]4C[C@H](n5cc(-c6cccc(N[C@H]7CC[C@@H](O)CC7)n6)c(C6CC6)n5)C4)cc3C2=O)C(=O)N1] | 5-(((trans-3-(3-cyclopropyl-4-(6-((cis-4- hydroxycyclohexyl)amino)pyridin-2-yl)- 1H-pyrazol-1- yl)cyclobutyl)methyl)amino)-2-(2,6- dioxopiperidin-3-yl)isoindoline-1,3-dione |
| GS-446 | 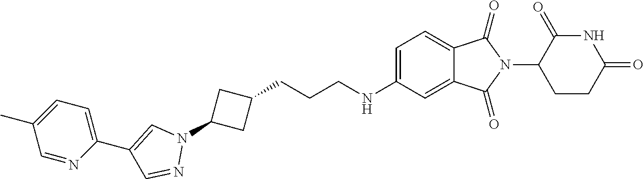 [SMILES: Cc1ccc(-c2cnn([C@H]3C[C@H](CCCNc4ccc5c(c4)C(=O)N(C4CCC(=O)NC4=O)C5=O)C3)c2)nc1] | 2-(2,6-dioxopiperidin-3-yl)-5-((3-(trans- 3-(4-(5-methylpyridin-2-yl)-1H-pyrazol- 1- yl)cyclobutyl)propyl)amino)isoindoline- 1,3-dione |
| GS-447 | 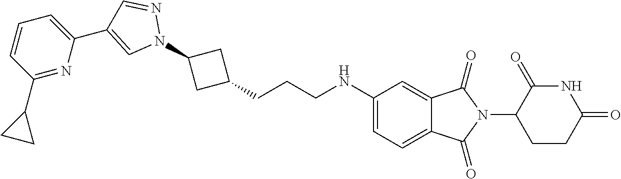 [SMILES: O=C1CCC(N2C(=O)c3ccc(NCCC[C@H]4C[C@H](n5cc(-c6cccc(C7CC7)n6)cn5)C4)cc3C2=O)C(=O)N1] | 5-((3-(trans-3-(4-(6-cyclopropylpyridin- 2-yl)-1H-pyrazol-1- yl)cyclobutyl)propyl)amino)-2-(2,6- dioxopiperidin-3-yl)isoindoline-1,3-dione |
| GS-448 | 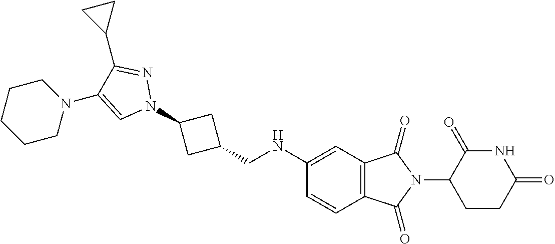 [SMILES: O=C1CCC(N2C(=O)c3ccc(NC[C@H]4C[C@H](n5cc(N6CCCCC6)c(C6CC6)n5)C4)cc3C2=O)C(=O)N1] | 5-(((trans-3-(3-cyclopropyl-4-(piperidin- 1-yl)-1H-pyrazol-1- yl)cyclobutyl)methyl)amino)-2-(2,6- dioxopiperidin-3-yl)isoindoline-1,3-dione |
| GS-449 | 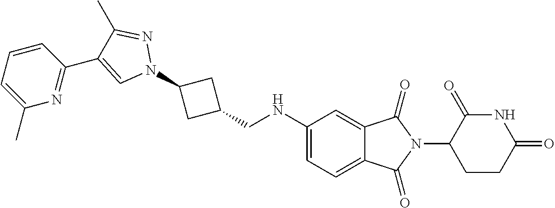 [SMILES: Cc1cccc(-c2cn([C@H]3C[C@H](CNc4ccc5c(c4)C(=O)N(C4CCC(=O)NC4=O)C5=O)C3)nc2C)n1] | 2-(2,6-dioxopiperidin-3-yl)-5-(((trans-3- (3-methyl-4-(6-methylpyridin-2-yl)-1H- pyrazol-1- yl)cyclobutyl)methyl)amino)isoindoline- 1,3-dione |
| GS-450 | 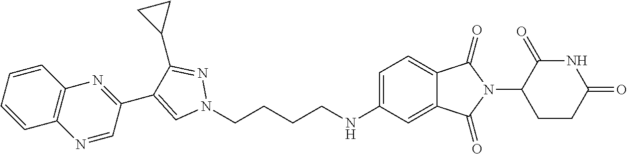 [SMILES: O=C1CCC(N2C(=O)c3ccc(NCCCCn4cc(-c5cnc6ccccc6n5)c(C5CC5)n4)cc3C2=O)C(=O)N1] | 5-((4-(3-cyclopropyl-4-(quinoxalin-2-yl)- 1H-pyrazol-1-yl)butyl)amino)-2-(2,6- dioxopiperidin-3-yl)isoindoline-1,3-dione |
| GS-451 | 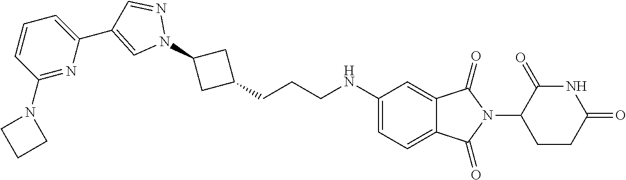 [SMILES: O=C1CCC(N2C(=O)c3ccc(NCCC[C@H]4C[C@H](n5cc(-c6cccc(N7CCC7)n6)cn5)C4)cc3C2=O)C(=O)N1] | 5-((3-(trans-3-(4-(6-(azetidin-1- yl)pyridin-2-yl)-1H-pyrazol-1- yl)cyclobutyl)propyl)amino)-2-(2,6- dioxopiperidin-3-yl)isoindoline-1,3-dione |
| GS-452 | 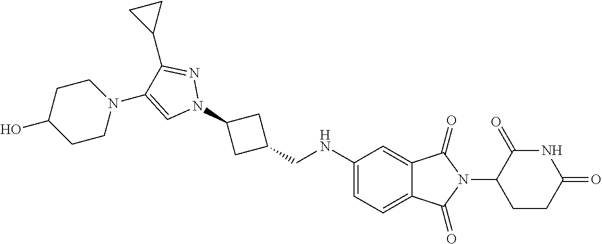 [SMILES: O=C1CCC(N2C(=O)c3ccc(NC[C@H]4C[C@H](n5cc(N6CCC(O)CC6)c(C6CC6)n5)C4)cc3C2=O)C(=O)N1] | 5-(((trans-3-(3-cyclopropyl-4-(4- hydroxypiperidin-1-yl)-1H-pyrazol-1- yl)cyclobutyl)methyl)amino)-2-(2,6- dioxopiperidin-3-yl)isoindoline-1,3-dione |
| GS-453 | 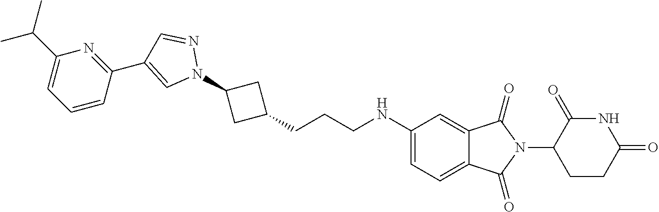 [SMILES: CC(C)c1cccc(-c2cnn([C@H]3C[C@H](CCCNc4ccc5c(c4)C(=O)N(C4CCC(=O)NC4=O)C5=O)C3)c2)n1] | 2-(2,6-dioxopiperidin-3-yl)-5-((3-(trans- 3-(4-(6-isopropylpyridin-2-yl)-1H- pyrazol-1- yl)cyclobutyl)propyl)amino)isoindoline- 1,3-dione |
| GS-454 | 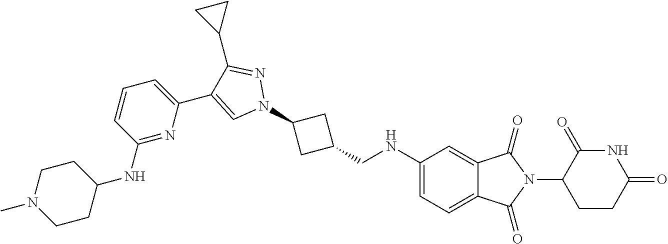 [SMILES: CN1CCC(Nc2cccc(-c3cn([C@H]4C[C@H](CNc5ccc6c(c5)C(=O)N(C5CCC(=O)NC5=O)C6=O)C4)nc3C3CC3)n2)CC1] | 5-(((trans-3-(3-cyclopropyl-4-(6-((1- methylpiperidin-4-yl)amino)pyridin-2- yl)-1H-pyrazol-1- yl)cyclobutyl)methyl)amino)-2-(2,6- dioxopiperidin-3-yl)isoindoline-1,3-dione |
| GS-455 | 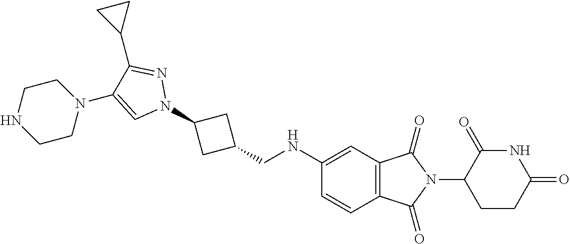 [SMILES: O=C1CCC(N2C(=O)c3ccc(NC[C@H]4C[C@H](n5cc(N6CCNCC6)c(C6CC6)n5)C4)cc3C2=O)C(=O)N1] | 5-(((trans-3-(3-cyclopropyl-4-(piperazin- 1-yl)-1H-pyrazol-1- yl)cyclobutyl)methyl)amino)-2-(2,6- dioxopiperidin-3-yl)isoindoline-1,3-dione |
| GS-456 | 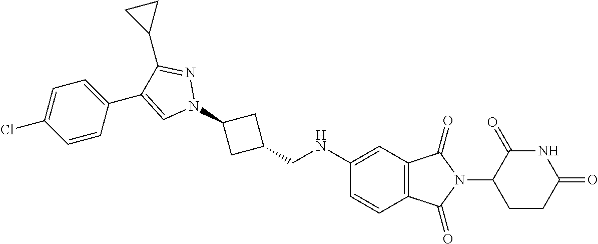 [SMILES: O=C1CCC(N2C(=O)c3ccc(NC[C@H]4C[C@H](n5cc(-c6ccc(Cl)cc6)c(C6CC6)n5)C4)cc3C2=O)C(=O)N1] | 5-(((trans-3-(4-(4-chlorophenyl)-3- cyclopropyl-1H-pyrazol-1- yl)cyclobutyl)methyl)amino)-2-(2,6- dioxopiperidin-3-yl)isoindoline-1,3-dione |
| GS-457 | 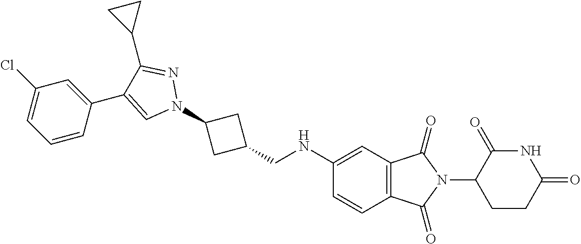 [SMILES: O=C1CCC(N2C(=O)c3ccc(NC[C@H]4C[C@H](n5cc(-c6cccc(Cl)c6)c(C6CC6)n5)C4)cc3C2=O)C(=O)N1] | 5-(((trans-3-(4-(3-chlorophenyl)-3- cyclopropyl-1H-pyrazol-1- yl)cyclobutyl)methyl)amino)-2-(2,6- dioxopiperidin-3-yl)isoindoline-1,3-dione |
| GS-458 | 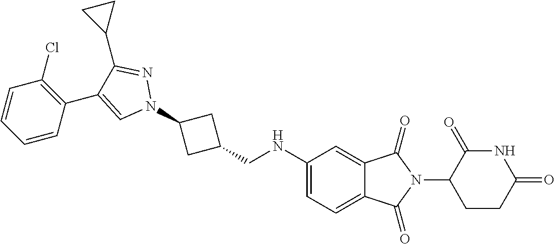 [SMILES: O=C1CCC(N2C(=O)c3ccc(NC[C@H]4C[C@H](n5cc(-c6ccccc6Cl)c(C6CC6)n5)C4)cc3C2=O)C(=O)N1] | 5-(((trans-3-(4-(2-chlorophenyl)-3- cyclopropyl-1H-pyrazol-1- yl)cyclobutyl)methyl)amino)-2-(2,6- dioxopiperidin-3-yl)isoindoline-1,3-dione |
| GS-459 | 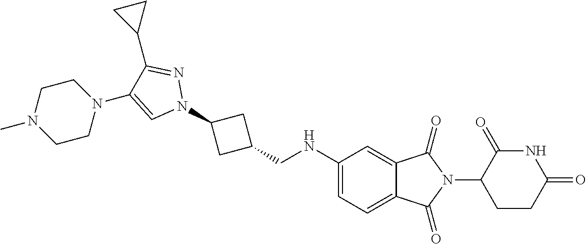 [SMILES: CN1CCN(c2cn([C@H]3C[C@H](CNc4ccc5c(c4)C(=O)N(C4CCC(=O)NC4=O)C5=O)C3)nc2C2CC2)CC1] | 5-(((trans-3-(3-cyclopropyl-4-(4- methylpiperazin-1-yl)-1H-pyrazol-1- yl)cyclobutyl)methyl)amino)-2-(2,6- dioxopiperidin-3-yl)isoindoline-1,3-dione |
| GS-460 | 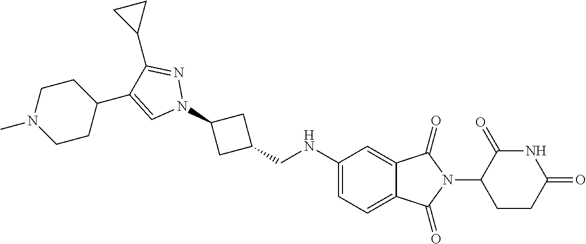 [SMILES: CN1CCC(c2cn([C@H]3C[C@H](CNc4ccc5c(c4)C(=O)N(C4CCC(=O)NC4=O)C5=O)C3)nc2C2CC2)CC1] | 5-(((trans-3-(3-cyclopropyl-4-(1- methylpiperidin-4-yl)-1H-pyrazol-1- yl)cyclobutyl)methyl)amino)-2-(2,6- dioxopiperidin-3-yl)isoindoline-1,3-dione |
| GS-461 | 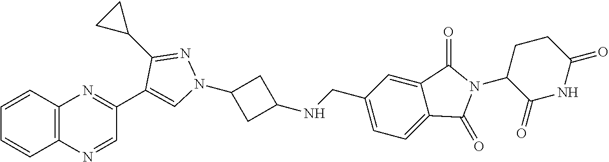 [SMILES: O=C1CCC(N2C(=O)c3ccc(CNC4CC(n5cc(-c6cnc7ccccc7n6)c(C6CC6)n5)C4)cc3C2=O)C(=O)N1] | 5-(((3-(3-cyclopropyl-4-(quinoxalin-2- yl)-1H-pyrazol-1- yl)cyclobutyl)amino)methyl)-2-(2,6- dioxopiperidin-3-yl)isoindoline-1,3-dione |
| GS-462 | 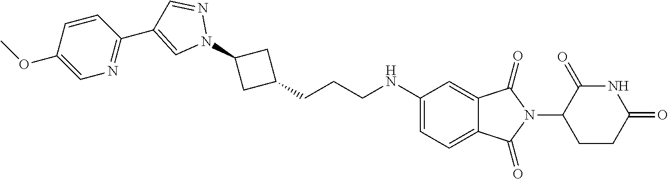 [SMILES: COc1ccc(-c2cnn([C@H]3C[C@H](CCCNc4ccc5c(c4)C(=O)N(C4CCC(=O)NC4=O)C5=O)C3)c2)nc1] | 2-(2,6-dioxopiperidin-3-yl)-5-((3-(trans- 3-(4-(5-methoxypyridin-2-yl)-1H- pyrazol-1- yl)cyclobutyl)propyl)amino)isoindoline- 1,3-dione |
| GS-463 | 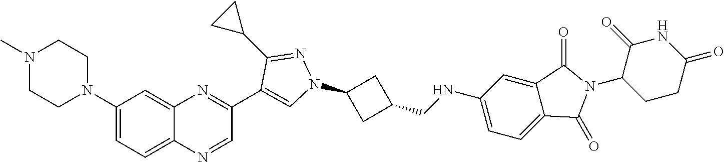 [SMILES: CN1CCN(c2ccc3ncc(-c4cn([C@H]5C[C@H](CNc6ccc7c(c6)C(=O)N(C6CCC(=O)NC6=O)C7=O)C5)nc4C4CC4)nc3c2)CC1] | 5-(((trans-3-(3-cyclopropyl-4-(7-(4- methylpiperazin-1-yl)quinoxalin-2-yl)- 1H-pyrazol-1- yl)cyclobutyl)methyl)amino)-2-(2,6- dioxopiperidin-3-yl)isoindoline-1,3-dione |
| GS-464 | 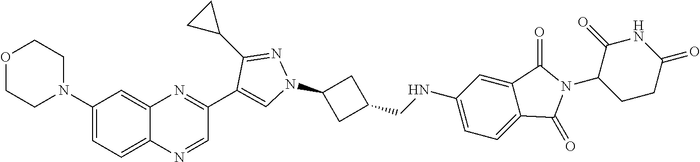 [SMILES: O=C1CCC(N2C(=O)c3ccc(NC[C@H]4C[C@H](n5cc(-c6cnc7ccc(N8CCOCC8)cc7n6)c(C6CC6)n5)C4)cc3C2=O)C(=O)N1] | 5-(((trans-3-(3-cyclopropyl-4-(7- morpholinoquinoxalin-2-yl)-1H-pyrazol- 1-yl)cyclobutyl)methyl)amino)-2-(2,6- dioxopiperidin-3-yl)isoindoline-1,3-dione |
| GS-465 | 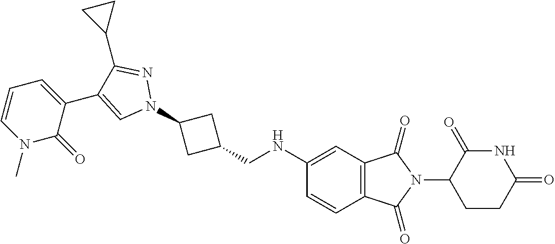 [SMILES: Cn1cccc(-c2cn([C@H]3C[C@H](CNc4ccc5c(c4)C(=O)N(C4CCC(=O)NC4=O)C5=O)C3)nc2C2CC2)c1=O] | 5-(((trans-3-(3-cyclopropyl-4-(1-methyl- 2-oxo-1,2-dihydropyridin-3-yl)-1H- pyrazol-1-yl)cyclobutyl)methyl)amino)- 2-(2,6-dioxopiperidin-3-yl)isoindoline- 1,3-dione |
| GS-466 | 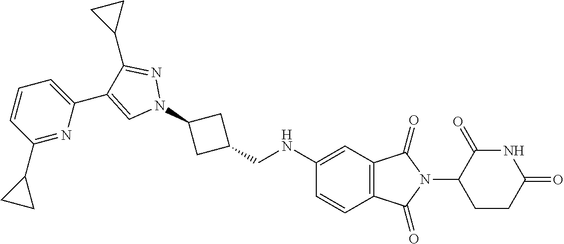 [SMILES: O=C1CCC(N2C(=O)c3ccc(NC[C@H]4C[C@H](n5cc(-c6cccc(C7CC7)n6)c(C6CC6)n5)C4)cc3C2=O)C(=O)N1] | 5-(((trans-3-(3-cyclopropyl-4-(6- cyclopropylpyridin-2-yl)-1H-pyrazol-1- yl)cyclobutyl)methyl)amino)-2-(2,6- dioxopiperidin-3-yl)isoindoline-1,3-dione |
| GS-467 | 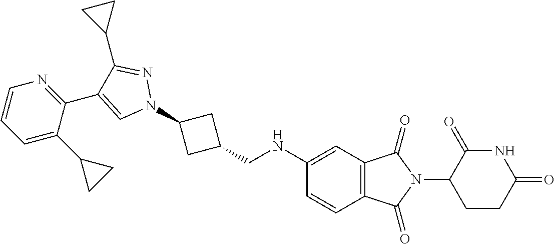 [SMILES: O=C1CCC(N2C(=O)c3ccc(NC[C@H]4C[C@H](n5cc(-c6ncccc6C6CC6)c(C6CC6)n5)C4)cc3C2=O)C(=O)N1] | 5-(((trans-3-(3-cyclopropyl-4-(3- cyclopropylpyridin-2-yl)-1H-pyrazol-1- yl)cyclobutyl)methyl)amino)-2-(2,6- dioxopiperidin-3-yl)isoindoline-1,3-dione |
| GS-468 | 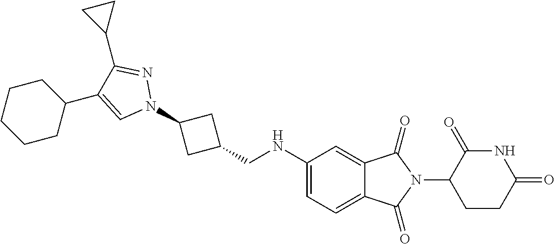 [SMILES: O=C1CCC(N2C(=O)c3ccc(NC[C@H]4C[C@H](n5cc(C6CCCCC6)c(C6CC6)n5)C4)cc3C2=O)C(=O)N1] | 5-(((trans-3-(4-cyclohexyl-3- cyclopropyl-1H-pyrazol-1- yl)cyclobutyl)methyl)amino)-2-(2,6- dioxopiperidin-3-yl)isoindoline-1,3-dione |
| GS-469 | 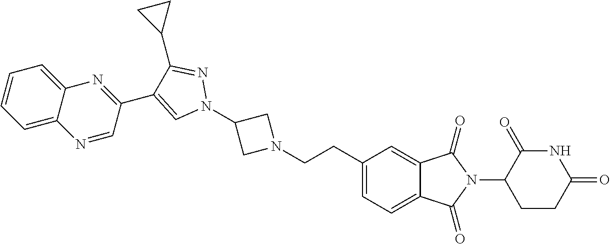 [SMILES: O=C1CCC(N2C(=O)c3ccc(CCN4CC(n5cc(-c6cnc7ccccc7n6)c(C6CC6)n5)C4)cc3C2=O)C(=O)N1] | 5-(2-(3-(3-cyclopropyl-4-(quinoxalin-2- yl)-1H-pyrazol-1-yl)azetidin-1-yl)ethyl)- 2-(2,6-dioxopiperidin-3-yl)isoindoline- 1,3-dione |
| GS-470 | 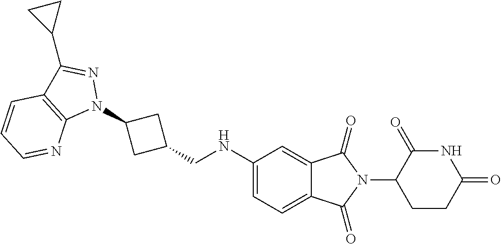 [SMILES: O=C1CCC(N2C(=O)c3ccc(NC[C@H]4C[C@H](n5nc(C6CC6)c6cccnc65)C4)cc3C2=O)C(=O)N1] | 5-(((trans-3-(3-cyclopropyl-1H- pyrazolo[3,4-b]pyridin-1- yl)cyclobutyl)methyl)amino)-2-(2,6- dioxopiperidin-3-yl)isoindoline-1,3-dione |
| GS-471 | 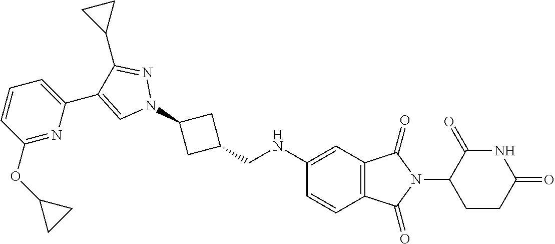 [SMILES: O=C1CCC(N2C(=O)c3ccc(NC[C@H]4C[C@H](n5cc(-c6cccc(OC7CC7)n6)c(C6CC6)n5)C4)cc3C2=O)C(=O)N1] | 5-(((trans-3-(4-(6-cyclopropoxypyridin- 2-yl)-3-cyclopropyl-1H-pyrazol-1- yl)cyclobutyl)methyl)amino)-2-(2,6- dioxopiperidin-3-yl)isoindoline-1,3-dione |
| GS-472 | 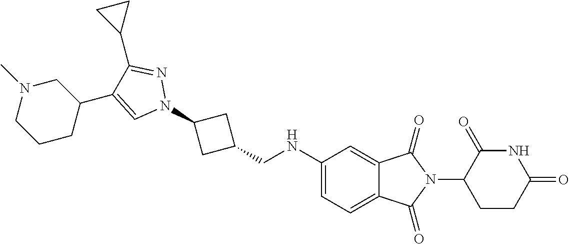 [SMILES: CN1CCCC(c2cn([C@H]3C[C@H](CNc4ccc5c(c4)C(=O)N(C4CCC(=O)NC4=O)C5=O)C3)nc2C2CC2)C1] | 5-(((trans-3-(3-cyclopropyl-4-(1- methylpiperidin-3-yl)-1H-pyrazol-1- yl)cyclobutyl)methyl)amino)-2-(2,6- dioxopiperidin-3-yl)isoindoline-1,3-dione |
| GS-473 | 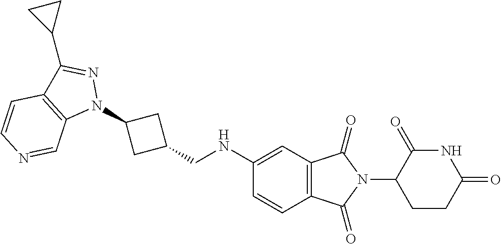 [SMILES: O=C1CCC(N2C(=O)c3ccc(NC[C@H]4C[C@H](n5nc(C6CC6)c6ccncc65)C4)cc3C2=O)C(=O)N1] | 5-(((trans-3-(3-cyclopropyl-1H- pyrazolo[3,4-c]pyridin-1- yl)cyclobutyl)methyl)amino)-2-(2,6- dioxopiperidin-3-yl)isoindoline-1,3-dione |
| GS-474 | 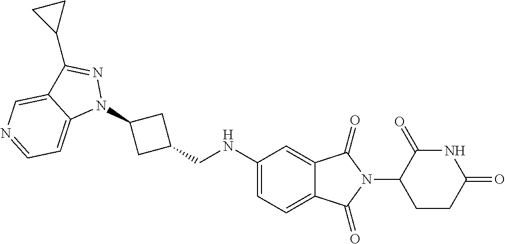 [SMILES: O=C1CCC(N2C(=O)c3ccc(NC[C@H]4C[C@H](n5nc(C6CC6)c6cnccc65)C4)cc3C2=O)C(=O)N1] | 5-(((trans-3-(3-cyclopropyl-1H- pyrazolo[4,3-c]pyridin-1- yl)cyclobutyl)methyl)amino)-2-(2,6- dioxopiperidin-3-yl)isoindoline-1,3-dione |
| GS-475 | 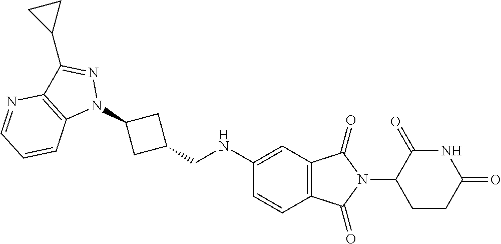 [SMILES: O=C1CCC(N2C(=O)c3ccc(NC[C@H]4C[C@H](n5nc(C6CC6)c6ncccc65)C4)cc3C2=O)C(=O)N1] | 5-(((trans-3-(3-cyclopropyl-1H- pyrazolo[4,3-b]pyridin-1- yl)cyclobutyl)methyl)amino)-2-(2,6- dioxopiperidin-3-yl)isoindoline-1,3-dione |
| GS-476 | 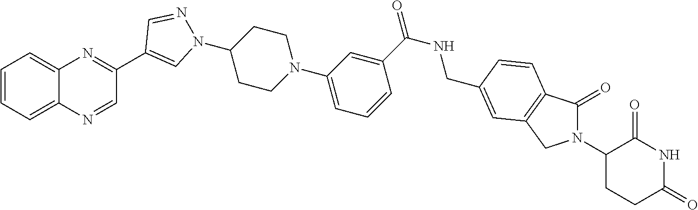 [SMILES: O=C1CCC(N2Cc3cc(CNC(=O)c4cccc(N5CCC(n6cc(-c7cnc8ccccc8n7)cn6)CC5)c4)ccc3C2=O)C(=O)N1] | N-((2-(2,6-dioxopiperidin-3-yl)-1- oxoisoindolin-5-yl)methyl)-3-(4-(4- (quinoxalin-2-yl)-1H-pyrazol-1- yl)piperidin-1-yl)benzamide |
| GS-477 | 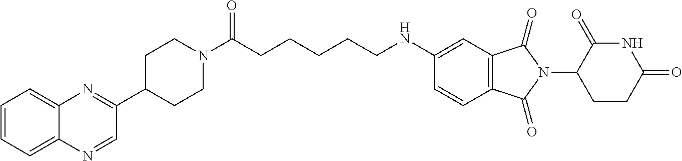 [SMILES: O=C1CCC(N2C(=O)c3ccc(NCCCCCC(=O)N4CCC(c5cnc6ccccc6n5)CC4)cc3C2=O)C(=O)N1] | 2-(2,6-dioxopiperidin-3-yl)-5-((6-oxo-6- (4-(quinoxalin-2-yl)piperidin-1- yl)hexyl)amino)isoindoline-1,3-dione |
| GS-478 | 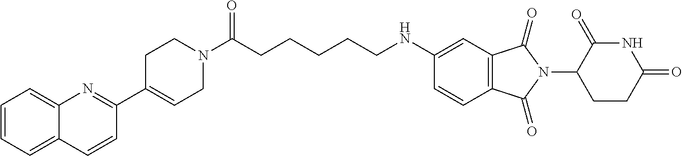 [SMILES: O=C1CCC(N2C(=O)c3ccc(NCCCCCC(=O)N4CC=C(c5ccc6ccccc6n5)CC4)cc3C2=O)C(=O)N1] | 2-(2,6-dioxopiperidin-3-yl)-5-((6-oxo-6- (4-(quinolin-2-yl)-3,6-dihydropyridin- 1(2H)-yl)hexyl)amino)isoindoline-1,3- dione |
| GS-479 | 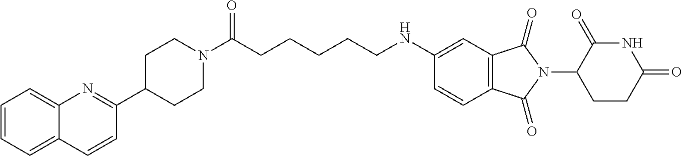 [SMILES: O=C1CCC(N2C(=O)c3ccc(NCCCCCC(=O)N4CCC(c5ccc6ccccc6n5)CC4)cc3C2=O)C(=O)N1] | 2-(2,6-dioxopiperidin-3-yl)-5-((6-oxo-6- (4-(quinolin-2-yl)piperidin-1- yl)hexyl)amino)isoindoline-1,3-dione |
| GS-480 | 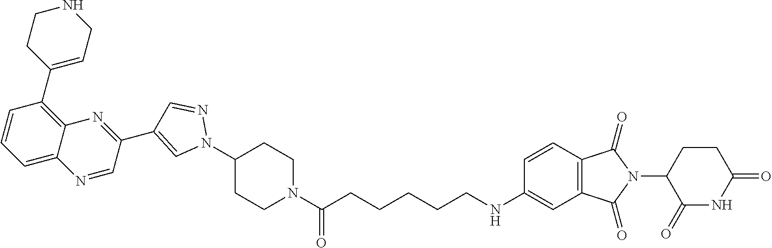 [SMILES: O=C1CCC(N2C(=O)c3ccc(NCCCCCC(=O)N4CCC(n5cc(-c6cnc7cccc(C8=CCNCC8)c7n6)cn5)CC4)cc3C2=O)C(=O)N1] | 2-(2,6-dioxopiperidin-3-yl)-5-((6-oxo-6- (4-(4-(8-(1,2,3,6-tetrahydropyridin-4- yl)quinoxalin-2-yl)-1H-pyrazol-1- yl)piperidin-1- yl)hexyl)amino)isoindoline-1,3-dione |
| GS-481 | 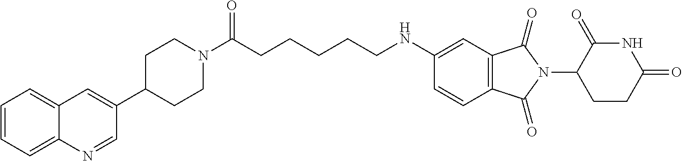 [SMILES: O=C1CCC(N2C(=O)c3ccc(NCCCCCC(=O)N4CCC(c5cnc6ccccc6c5)CC4)cc3C2=O)C(=O)N1] | 2-(2,6-dioxopiperidin-3-yl)-5-((6-oxo-6- (4-(quinolin-3-yl)piperidin-1- yl)hexyl)amino)isoindoline-1,3-dione |
| GS-482 | 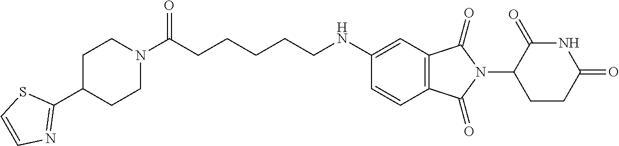 [SMILES: O=C1CCC(N2C(=O)c3ccc(NCCCCCC(=O)N4CCC(c5nccs5)CC4)cc3C2=O)C(=O)N1] | 2-(2,6-dioxopiperidin-3-yl)-5-((6-oxo-6- (4-(thiazol-2-yl)piperidin-1- yl)hexyl)amino)isoindoline-1,3-dione |
| GS-483 | 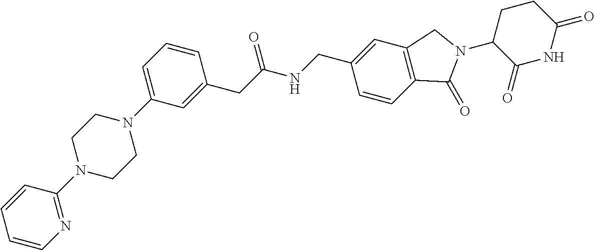 [SMILES: O=C(Cc1cccc(N2CCN(c3ccccn3)CC2)c1)NCc1ccc2c(c1)CN(C1CCC(=O)NC1=O)C2=O] | N-((2-(2,6-dioxopiperidin-3-yl)-1- oxoisoindolin-5-yl)methyl)-2-(3-(4- (pyridin-2-yl)piperazin-1- yl)phenyl)acetamide |
| GS-484 | 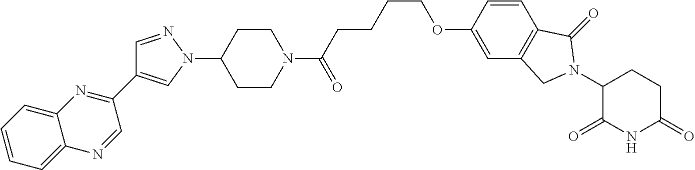 [SMILES: O=C1CCC(N2Cc3cc(OCCCCC(=O)N4CCC(n5cc(-c6cnc7ccccc7n6)cn5)CC4)ccc3C2=O)C(=O)N1] | 3-(1-oxo-5-((5-oxo-5-(4-(4-(quinoxalin- 2-yl)-1H-pyrazol-1-yl)piperidin-1- yl)pentyl)oxy)isoindolin-2-yl)piperidine- 2,6-dione |
| GS-485 | 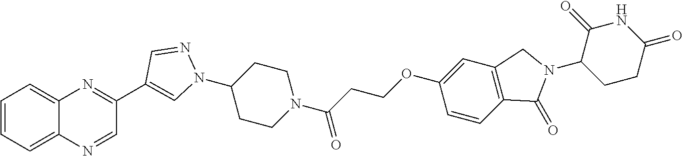 [SMILES: O=C1CCC(N2Cc3cc(OCCC(=O)N4CCC(n5cc(-c6cnc7ccccc7n6)cn5)CC4)ccc3C2=O)C(=O)N1] | 3-(1-oxo-5-(3-oxo-3-(4-(4-(quinoxalin-2- yl)-1H-pyrazol-1-yl)piperidin-1- yl)propoxy)isoindolin-2-yl)piperidine- 2,6-dione |
| GS-486 | 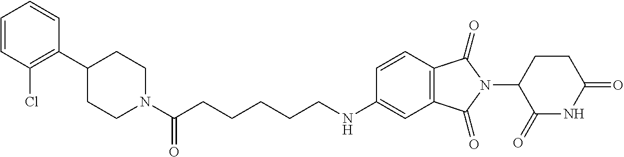 [SMILES: O=C1CCC(N2C(=O)c3ccc(NCCCCCC(=O)N4CCC(c5ccccc5Cl)CC4)cc3C2=O)C(=O)N1] | 5-((6-(4-(2-chlorophenyl)piperidin-1-yl)- 6-oxohexyl)amino)-2-(2,6- dioxopiperidin-3-yl)isoindoline-1,3-dione |
| GS-487 | 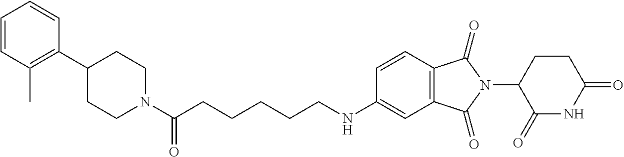 [SMILES: Cc1ccccc1C1CCN(C(=O)CCCCCNc2ccc3c(c2)C(=O)N(C2CCC(=O)NC2=O)C3=O)CC1] | 2-(2,6-dioxopiperidin-3-yl)-5-((6-oxo-6- (4-(o-tolyl)piperidin-1- yl)hexyl)amino)isoindoline-1,3-dione |
| GS-488 | 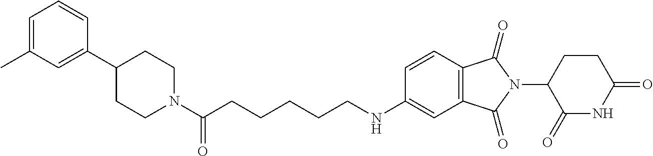 [SMILES: Cc1cccc(C2CCN(C(=O)CCCCCNc3ccc4c(c3)C(=O)N(C3CCC(=O)NC3=O)C4=O)CC2)c1] | 2-(2,6-dioxopiperidin-3-yl)-5-((6-oxo-6- (4-(m-tolyl)piperidin-1- yl)hexyl)amino)isoindoline-1,3-dione |
| GS-489 | 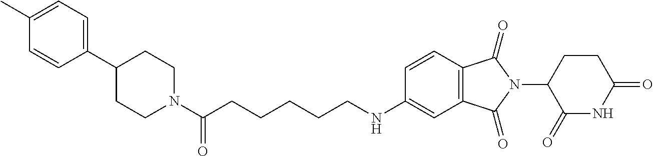 [SMILES: Cc1ccc(C2CCN(C(=O)CCCCCNc3ccc4c(c3)C(=O)N(C3CCC(=O)NC3=O)C4=O)CC2)cc1] | 2-(2,6-dioxopiperidin-3-yl)-5-((6-oxo-6- (4-(p-tolyl)piperidin-1- yl)hexyl)amino)isoindoline-1,3-dione |
| GS-490 | 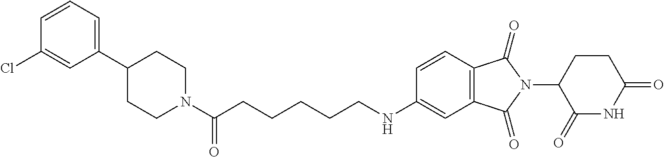 [SMILES: O=C1CCC(N2C(=O)c3ccc(NCCCCCC(=O)N4CCC(c5cccc(Cl)c5)CC4)cc3C2=O)C(=O)N1] | 5-((6-(4-(3-chlorophenyl)piperidin-1-yl)- 6-oxohexyl)amino)-2-(2,6- dioxopiperidin-3-yl)isoindoline-1,3-dione |
| GS-491 | 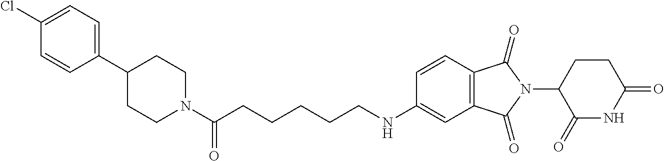 [SMILES: O=C1CCC(N2C(=O)c3ccc(NCCCCCC(=O)N4CCC(c5ccc(Cl)cc5)CC4)cc3C2=O)C(=O)N1] | 5-((6-(4-(4-chlorophenyl)piperidin-1-yl)- 6-oxohexyl)amino)-2-(2,6- dioxopiperidin-3-yl)isoindoline-1,3-dione |
| GS-492 | 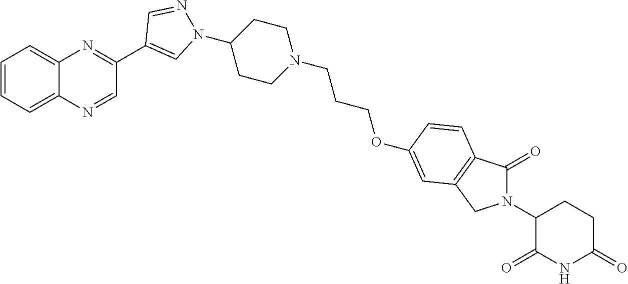 [SMILES: O=C1CCC(N2Cc3cc(OCCCN4CCC(n5cc(-c6cnc7ccccc7n6)cn5)CC4)ccc3C2=O)C(=O)N1] | 3-(1-oxo-5-(3-(4-(4-(quinoxalin-2-yl)- 1H-pyrazol-1-yl)piperidin-1- yl)propoxy)isoindolin-2-yl)piperidine- 2,6-dione |
| GS-493 | 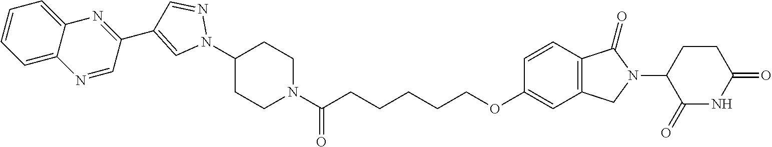 [SMILES: O=C1CCC(N2Cc3cc(OCCCCCC(=O)N4CCC(n5cc(-c6cnc7ccccc7n6)cn5)CC4)ccc3C2=O)C(=O)N1] | 3-(1-oxo-5-((6-oxo-6-(4-(4-(quinoxalin- 2-yl)-1H-pyrazol-1-yl)piperidin-1- yl)hexyl)oxy)isoindolin-2-yl)piperidine- 2,6-dione |
| GS-494 | 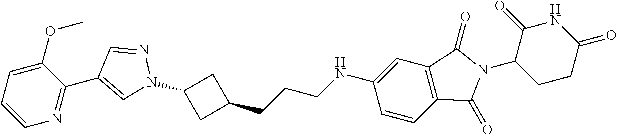 [SMILES: COc1cccnc1-c1cnn([C@H]2C[C@H](CCCNc3ccc4c(c3)C(=O)N(C3CCC(=O)NC3=O)C4=O)C2)c1] | 2-(2,6-dioxopiperidin-3-yl)-5-((3-(trans- 3-(4-(3-methoxypyridin-2-yl)-1H- pyrazol-1- yl)cyclobutyl)propyl)amino)isoindoline- 1,3-dione |
| GS-495 | 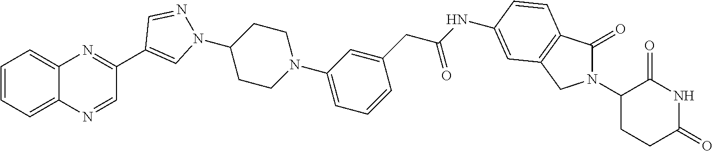 [SMILES: O=C1CCC(N2Cc3cc(NC(=O)Cc4cccc(N5CCC(n6cc(-c7cnc8ccccc8n7)cn6)CC5)c4)ccc3C2=O)C(=O)N1] | N-(2-(2,6-dioxopiperidin-3-yl)-1- oxoisoindolin-5-yl)-2-(3-(4-(4- (quinoxalin-2-yl)-1H-pyrazol-1- yl)piperidin-1-yl)phenyl)acetamide |
| GS-496 | 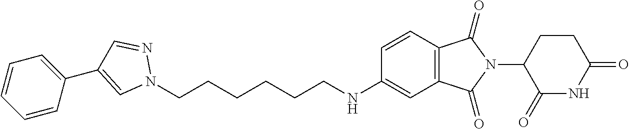 [SMILES: O=C1CCC(N2C(=O)c3ccc(NCCCCCCn4cc(-c5ccccc5)cn4)cc3C2=O)C(=O)N1] | 2-(2,6-dioxopiperidin-3-yl)-5-((6-(4- phenyl-1H-pyrazol-1- yl)hexyl)amino)isoindoline-1,3-dione |
| GS-497 | 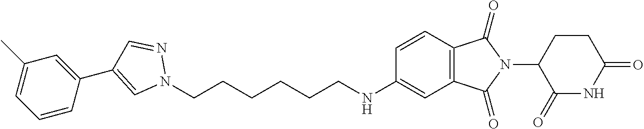 [SMILES: Cc1cccc(-c2cnn(CCCCCCNc3ccc4c(c3)C(=O)N(C3CCC(=O)NC3=O)C4=O)c2)c1] | 2-(2,6-dioxopiperidin-3-yl)-5-((6-(4-(m- tolyl)-1H-pyrazol-1- yl)hexyl)amino)isoindoline-1,3-dione |
| GS-498 | 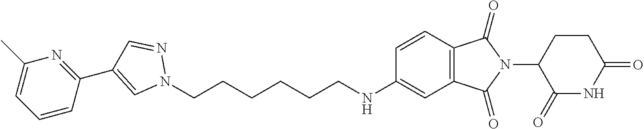 [SMILES: Cc1cccc(-c2cnn(CCCCCCNc3ccc4c(c3)C(=O)N(C3CCC(=O)NC3=O)C4=O)c2)n1] | 2-(2,6-dioxopiperidin-3-yl)-5-((6-(4-(6- methylpyridin-2-yl)-1H-pyrazol-1- yl)hexyl)amino)isoindoline-1,3-dione |
| GS-499 | 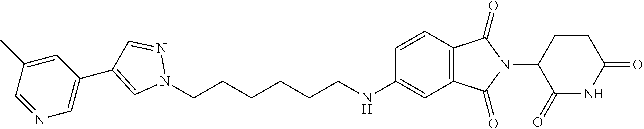 [SMILES: Cc1cncc(-c2cnn(CCCCCCNc3ccc4c(c3)C(=O)N(C3CCC(=O)NC3=O)C4=O)c2)c1] | 2-(2,6-dioxopiperidin-3-yl)-5-((6-(4-(5- methylpyridin-3-yl)-1H-pyrazol-1- yl)hexyl)amino)isoindoline-1,3-dione |
| GS-500 | 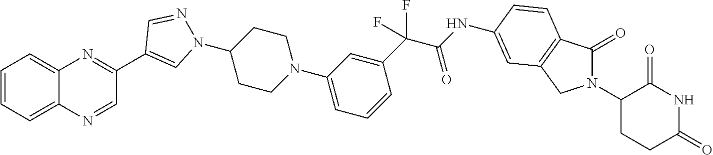 [SMILES: O=C1CCC(N2Cc3cc(NC(=O)C(F)(F)c4cccc(N5CCC(n6cc(-c7cnc8ccccc8n7)cn6)CC5)c4)ccc3C2=O)C(=O)N1] | N-(2-(2,6-dioxopiperidin-3-yl)-1- oxoisoindolin-5-yl)-2,2-difluoro-2-(3-(4- (4-(quinoxalin-2-yl)-1H-pyrazol-1- yl)piperidin-1-yl)phenyl)acetamide |
| GS-501 | 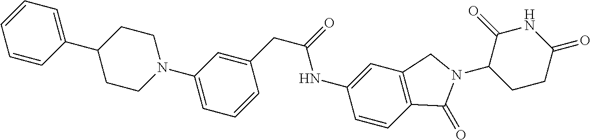 [SMILES: O=C1CCC(N2Cc3cc(NC(=O)Cc4cccc(N5CCC(c6ccccc6)CC5)c4)ccc3C2=O)C(=O)N1] | N-(2-(2,6-dioxopiperidin-3-yl)-1- oxoisoindolin-5-yl)-2-(3-(4- phenylpiperidin-1-yl)phenyl)acetamide |
| GS-502 | 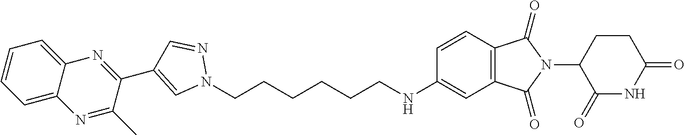 [SMILES: Cc1nc2ccccc2nc1-c1cnn(CCCCCCNc2ccc3c(c2)C(=O)N(C2CCC(=O)NC2=O)C3=O)c1] | 2-(2,6-dioxopiperidin-3-yl)-5-((6-(4-(3- methylquinoxalin-2-yl)-1H-pyrazol-1- yl)hexyl)amino)isoindoline-1,3-dione |
| GS-503 | 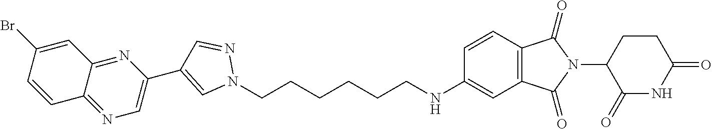 [SMILES: O=C1CCC(N2C(=O)c3ccc(NCCCCCCn4cc(-c5cnc6ccc(Br)cc6n5)cn4)cc3C2=O)C(=O)N1] | 5-((6-(4-(7-bromoquinoxalin-2-yl)-1H- pyrazol-1-yl)hexyl)amino)-2-(2,6- dioxopiperidin-3-yl)isoindoline-1,3-dione |
| GS-504 | 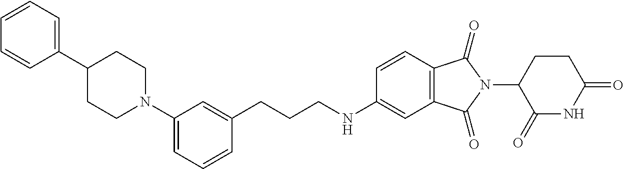 [SMILES: O=C1CCC(N2C(=O)c3ccc(NCCCc4cccc(N5CCC(c6ccccc6)CC5)c4)cc3C2=O)C(=O)N1] | 2-(2,6-dioxopiperidin-3-yl)-5-((3-(3-(4- phenylpiperidin-1- yl)phenyl)propyl)amino)isoindoline-1,3- dione |
| GS-505 | 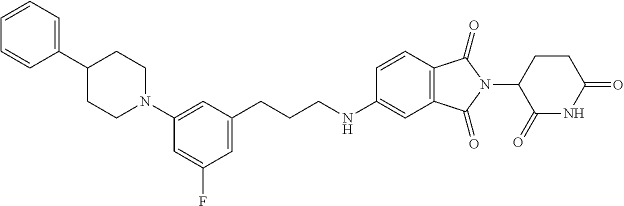 [SMILES: O=C1CCC(N2C(=O)c3ccc(NCCCc4cc(F)cc(N5CCC(c6ccccc6)CC5)c4)cc3C2=O)C(=O)N1] | 2-(2,6-dioxopiperidin-3-yl)-5-((3-(3- fluoro-5-(4-phenylpiperidin-1- yl)phenyl)propyl)amino)isoindoline-1,3- dione |
| GS-506 | 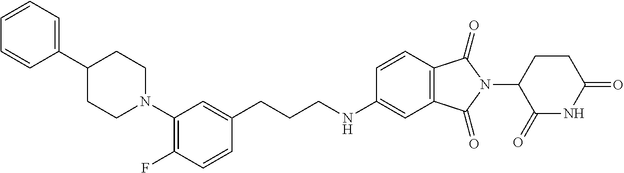 [SMILES: O=C1CCC(N2C(=O)c3ccc(NCCCc4ccc(F)c(N5CCC(c6ccccc6)CC5)c4)cc3C2=O)C(=O)N1] | 2-(2,6-dioxopiperidin-3-yl)-5-((3-(4- fluoro-3-(4-phenylpiperidin-1- yl)phenyl)propyl)amino)isoindoline-1,3- dione |
| GS-507 | 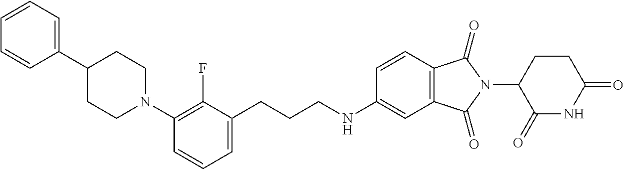 [SMILES: O=C1CCC(N2C(=O)c3ccc(NCCCc4cccc(N5CCC(c6ccccc6)CC5)c4F)cc3C2=O)C(=O)N1] | 2-(2,6-dioxopiperidin-3-yl)-5-((3-(2- fluoro-3-(4-phenylpiperidin-1- yl)phenyl)propyl)amino)isoindoline-1,3- dione |
| GS-508 | 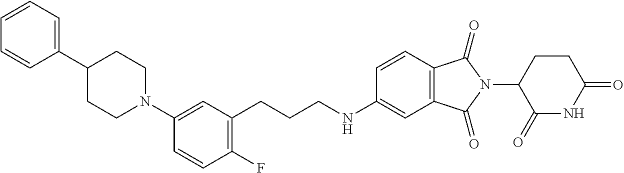 [SMILES: O=C1CCC(N2C(=O)c3ccc(NCCCc4cc(N5CCC(c6ccccc6)CC5)ccc4F)cc3C2=O)C(=O)N1] | 2-(2,6-dioxopiperidin-3-yl)-5-((3-(2- fluoro-5-(4-phenylpiperidin-1- yl)phenyl)propyl)amino)isoindoline-1,3- dione |
| GS-509 | 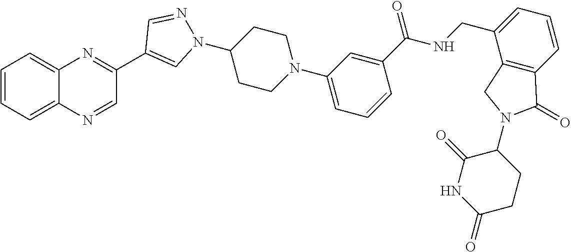 [SMILES: O=C1CCC(N2Cc3c(CNC(=O)c4cccc(N5CCC(n6cc(-c7cnc8ccccc8n7)cn6)CC5)c4)cccc3C2=O)C(=O)N1] | N-((2-(2,6-dioxopiperidin-3-yl)-1- oxoisoindolin-4-yl)methyl)-3-(4-(4- (quinoxalin-2-yl)-1H-pyrazol-1- yl)piperidin-1-yl)benzamide |
| GS-510 | 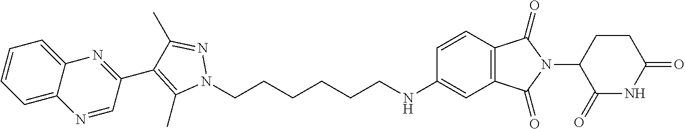 [SMILES: Cc1nn(CCCCCCNc2ccc3c(c2)C(=O)N(C2CCC(=O)NC2=O)C3=O)c(C)c1-c1cnc2ccccc2n1] | 5-((6-(3,5-dimethyl-4-(quinoxalin-2-yl)- 1H-pyrazol-1-yl)hexyl)amino)-2-(2,6- dioxopiperidin-3-yl)isoindoline-1,3-dione |
| GS-511 | 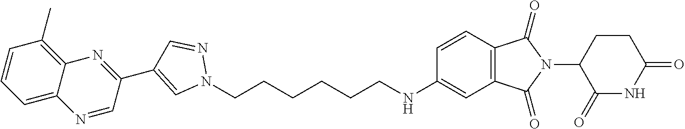 [SMILES: Cc1cccc2ncc(-c3cnn(CCCCCCNc4ccc5c(c4)C(=O)N(C4CCC(=O)NC4=O)C5=O)c3)nc12] | 2-(2,6-dioxopiperidin-3-yl)-5-((6-(4-(8- methylquinoxalin-2-yl)-1H-pyrazol-1- yl)hexyl)amino)isoindoline-1,3-dione |
| GS-512 | 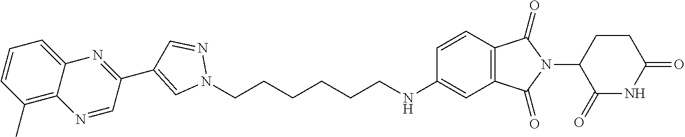 [SMILES: Cc1cccc2nc(-c3cnn(CCCCCCNc4ccc5c(c4)C(=O)N(C4CCC(=O)NC4=O)C5=O)c3)cnc12] | 2-(2,6-dioxopiperidin-3-yl)-5-((6-(4-(5- methylquinoxalin-2-yl)-1H-pyrazol-1- yl)hexyl)amino)isoindoline-1,3-dione |
| GS-513 | 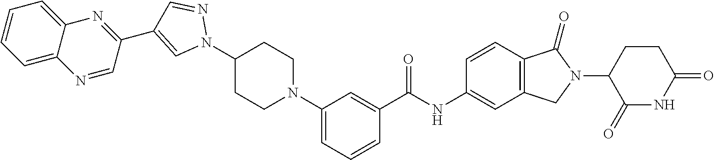 [SMILES: O=C1CCC(N2Cc3cc(NC(=O)c4cccc(N5CCC(n6cc(-c7cnc8ccccc8n7)cn6)CC5)c4)ccc3C2=O)C(=O)N1] | N-(2-(2,6-dioxopiperidin-3-yl)-1- oxoisoindolin-5-yl)-3-(4-(4-(quinoxalin- 2-yl)-1H-pyrazol-1-yl)piperidin-1- yl)benzamide |
| GS-514 | 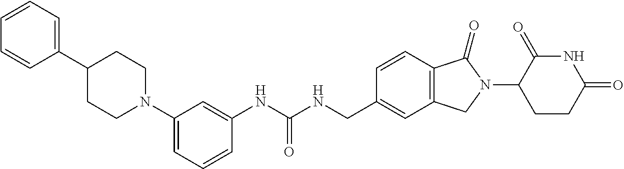 [SMILES: O=C1CCC(N2Cc3cc(CNC(=O)Nc4cccc(N5CCC(c6ccccc6)CC5)c4)ccc3C2=O)C(=O)N1] | 1-((2-(2,6-dioxopiperidin-3-yl)-1- oxoisoindolin-5-yl)methyl)-3-(3-(4- phenylpiperidin-1-yl)phenyl)urea |
| GS-515 | 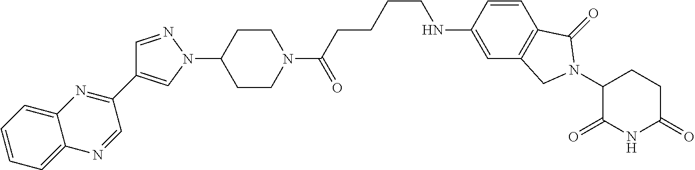 [SMILES: O=C1CCC(N2Cc3cc(NCCCCC(=O)N4CCC(n5cc(-c6cnc7ccccc7n6)cn5)CC4)ccc3C2=O)C(=O)N1] | 3-(1-oxo-5-((5-oxo-5-(4-(4-(quinoxalin- 2-yl)-1H-pyrazol-1-yl)piperidin-1- yl)pentyl)amino)isoindolin-2- yl)piperidine-2,6-dione |
| GS-516 | 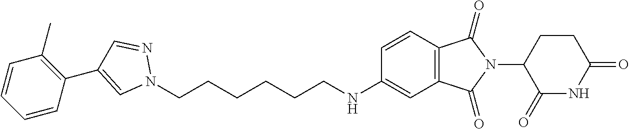 [SMILES: Cc1ccccc1-c1cnn(CCCCCCNc2ccc3c(c2)C(=O)N(C2CCC(=O)NC2=O)C3=O)c1] | 2-(2,6-dioxopiperidin-3-yl)-5-((6-(4-(o- tolyl)-1H-pyrazol-1- yl)hexyl)amino)isoindoline-1,3-dione |
| GS-517 | 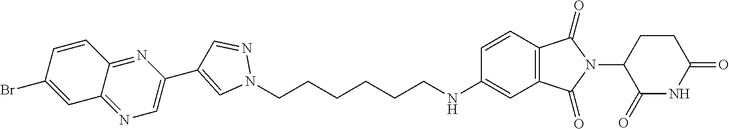 [SMILES: O=C1CCC(N2C(=O)c3ccc(NCCCCCCn4cc(-c5cnc6cc(Br)ccc6n5)cn4)cc3C2=O)C(=O)N1] | 5-((6-(4-(6-bromoquinoxalin-2-yl)-1H- pyrazol-1-yl)hexyl)amino)-2-(2,6- dioxopiperidin-3-yl)isoindoline-1,3-dione |
| GS-518 | 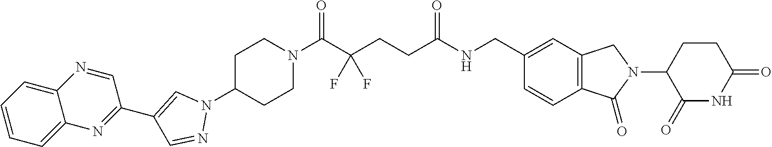 [SMILES: O=C(CCC(F)(F)C(=O)N1CCC(n2cc(-c3cnc4ccccc4n3)cn2)CC1)NCc1ccc2c(c1)CN(C1CCC(=O)NC1=O)C2=O] | N-((2-(2,6-dioxopiperidin-3-yl)-1- oxoisoindolin-5-yl)methyl)-4,4-difluoro- 5-oxo-5-(4-(4-(quinoxalin-2-yl)-1H- pyrazol-1-yl)piperidin-1-yl)pentanamide |
| GS-519 | 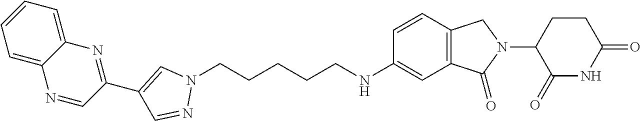 [SMILES: O=C1CCC(N2Cc3ccc(NCCCCCn4cc(-c5cnc6ccccc6n5)cn4)cc3C2=O)C(=O)N1] | 3-(1-oxo-6-((5-(4-(quinoxalin-2-yl)-1H- pyrazol-1-yl)pentyl)amino)isoindolin-2- yl)piperidine-2,6-dione |
| GS-520 | 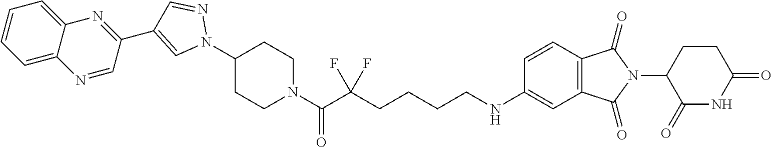 [SMILES: O=C1CCC(N2C(=O)c3ccc(NCCCCC(F)(F)C(=O)N4CCC(n5cc(-c6cnc7ccccc7n6)cn5)CC4)cc3C2=O)C(=O)N1] | 5-((5,5-difluoro-6-oxo-6-(4-(4- (quinoxalin-2-yl)-1H-pyrazol-1- yl)piperidin-1-yl)hexyl)amino)-2-(2,6- dioxopiperidin-3-yl)isoindoline-1,3-dione |
| GS-521 | 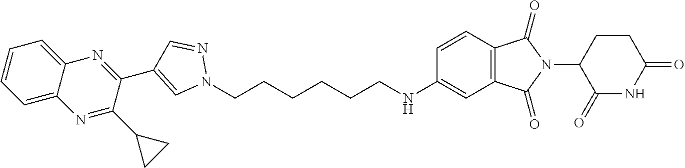 [SMILES: O=C1CCC(N2C(=O)c3ccc(NCCCCCCn4cc(-c5nc6ccccc6nc5C5CC5)cn4)cc3C2=O)C(=O)N1] | 5-((6-(4-(3-cyclopropylquinoxalin-2-yl)- 1H-pyrazol-1-yl)hexyl)amino)-2-(2,6- dioxopiperidin-3-yl)isoindoline-1,3-dione |
| GS-522 | 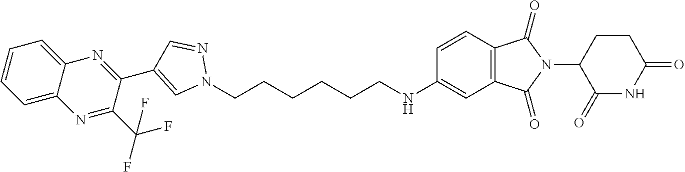 [SMILES: O=C1CCC(N2C(=O)c3ccc(NCCCCCCn4cc(-c5nc6ccccc6nc5C(F)(F)F)cn4)cc3C2=O)C(=O)N1] | 2-(2,6-dioxopiperidin-3-yl)-5-((6-(4-(3- (trifluoromethyl)quinoxalin-2-yl)-1H- pyrazol-1-yl)hexyl)amino)isoindoline- 1,3-dione |
| GS-523 | 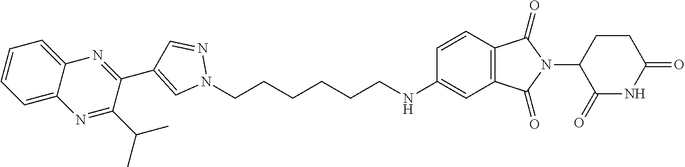 [SMILES: CC(C)c1nc2ccccc2nc1-c1cnn(CCCCCCNc2ccc3c(c2)C(=O)N(C2CCC(=O)NC2=O)C3=O)c1] | 2-(2,6-dioxopiperidin-3-yl)-5-((6-(4-(3- isopropylquinoxalin-2-yl)-1H-pyrazol-1- yl)hexyl)amino)isoindoline-1,3-dione |
| GS-524 | 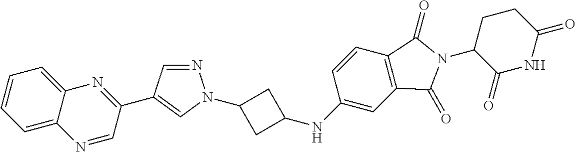 [SMILES: O=C1CCC(N2C(=O)c3ccc(NC4CC(n5cc(-c6cnc7ccccc7n6)cn5)C4)cc3C2=O)C(=O)N1] | 2-(2,6-dioxopiperidin-3-yl)-5-((3-(4- (quinoxalin-2-yl)-1H-pyrazol-1- yl)cyclobutyl)amino)isoindoline-1,3- dione |
| GS-525 | 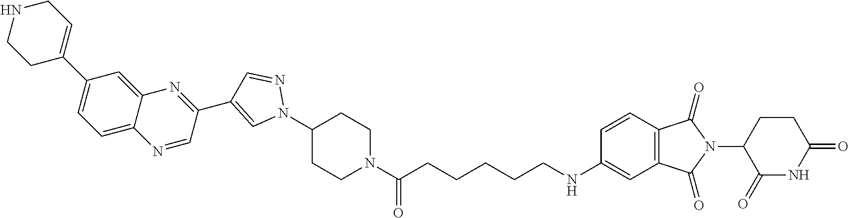 [SMILES: O=C1CCC(N2C(=O)c3ccc(NCCCCCC(=O)N4CCC(n5cc(-c6cnc7ccc(C8=CCNCC8)cc7n6)cn5)CC4)cc3C2=O)C(=O)N1] | 2-(2,6-dioxopiperidin-3-yl)-5-((6-oxo-6- (4-(4-(7-(1,2,3,6-tetrahydropyridin-4- yl)quinoxalin-2-yl)-1H-pyrazol-1- yl)piperidin-1- yl)hexyl)amino)isoindoline-1,3-dione |
| GS-526 | 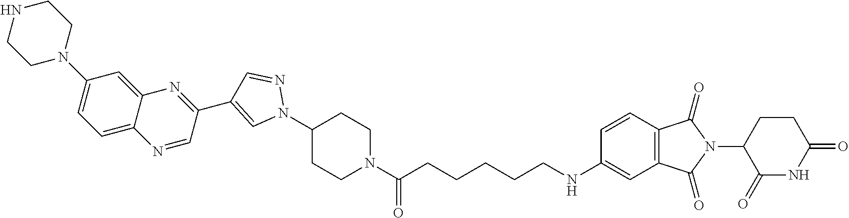 [SMILES: O=C1CCC(N2C(=O)c3ccc(NCCCCCC(=O)N4CCC(n5cc(-c6cnc7ccc(N8CCNCC8)cc7n6)cn5)CC4)cc3C2=O)C(=O)N1] | 2-(2,6-dioxopiperidin-3-yl)-5-((6-oxo-6- (4-(4-(7-(piperazin-1-yl)quinoxalin-2-yl)- 1H-pyrazol-1-yl)piperidin-1- yl)hexyl)amino)isoindoline-1,3-dione |
| GS-527 | 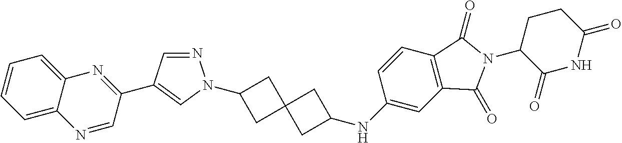 [SMILES: O=C1CCC(N2C(=O)c3ccc(NC4CC5(C4)CC(n4cc(-c6cnc7ccccc7n6)cn4)C5)cc3C2=O)C(=O)N1] | 2-(2,6-dioxopiperidin-3-yl)-5-((6-(4- (quinoxalin-2-yl)-1H-pyrazol-1- yl)spiro[3.3]heptan-2- yl)amino)isoindoline-1,3-dione |
| GS-528 | 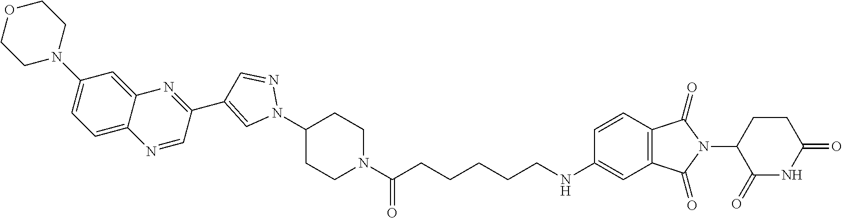 [SMILES: O=C1CCC(N2C(=O)c3ccc(NCCCCCC(=O)N4CCC(n5cc(-c6cnc7ccc(N8CCOCC8)cc7n6)cn5)CC4)cc3C2=O)C(=O)N1] | 2-(2,6-dioxopiperidin-3-yl)-5-((6-(4-(4- (7-morpholinoquinoxalin-2-yl)-1H- pyrazol-1-yl)piperidin-1-yl)-6- oxohexyl)amino)isoindoline-1,3-dione |
| GS-529 | 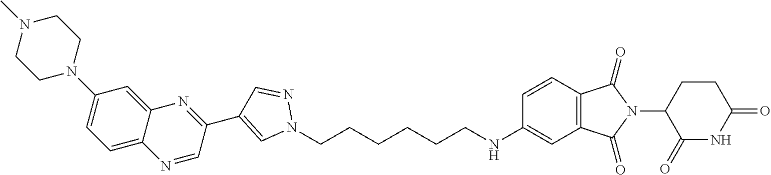 [SMILES: CN1CCN(c2ccc3ncc(-c4cnn(CCCCCCNc5ccc6c(c5)C(=O)N(C5CCC(=O)NC5=O)C6=O)c4)nc3c2)CC1] | 2-(2,6-dioxopiperidin-3-yl)-5-((6-(4-(7- (4-methylpiperazin-1-yl)quinoxalin-2-yl)- 1H-pyrazol-1- yl)hexyl)amino)isoindoline-1,3-dione |
| GS-530 | 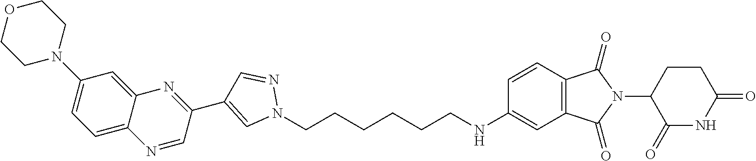 [SMILES: O=C1CCC(N2C(=O)c3ccc(NCCCCCCn4cc(-c5cnc6ccc(N7CCOCC7)cc6n5)cn4)cc3C2=O)C(=O)N1] | 2-(2,6-dioxopiperidin-3-yl)-5-((6-(4-(7- morpholinoquinoxalin-2-yl)-1H-pyrazol- 1-yl)hexyl)amino)isoindoline-1,3-dione |
| GS-531 | 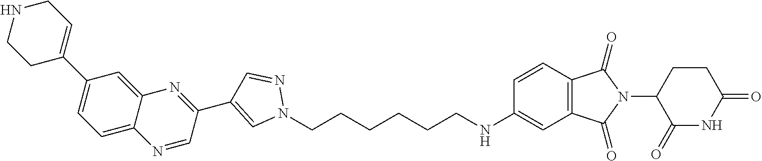 [SMILES: O=C1CCC(N2C(=O)c3ccc(NCCCCCCn4cc(-c5cnc6ccc(C7=CCNCC7)cc6n5)cn4)cc3C2=O)C(=O)N1] | 2-(2,6-dioxopiperidin-3-yl)-5-((6-(4-(7- (1,2,3,6-tetrahydropyridin-4- yl)quinoxalin-2-yl)-1H-pyrazol-1- yl)hexyl)amino)isoindoline-1,3-dione |
| GS-532 | 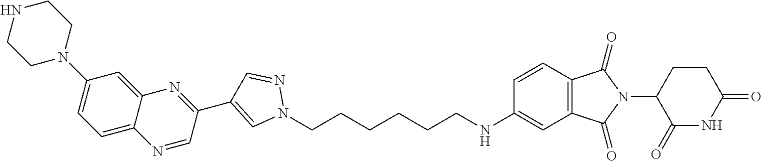 [SMILES: O=C1CCC(N2C(=O)c3ccc(NCCCCCCn4cc(-c5cnc6ccc(N7CCNCC7)cc6n5)cn4)cc3C2=O)C(=O)N1] | 2-(2,6-dioxopiperidin-3-yl)-5-((6-(4-(7- (piperazin-1-yl)quinoxalin-2-yl)-1H- pyrazol-1-yl)hexyl)amino)isoindoline- 1,3-dione |
| GS-533 | 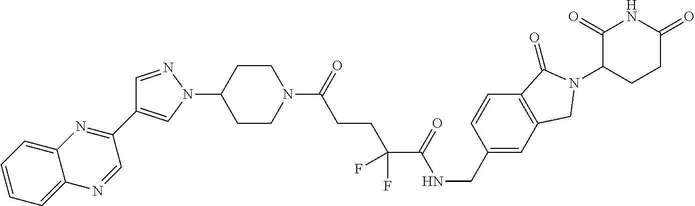 [SMILES: O=C1CCC(N2Cc3cc(CNC(=O)C(F)(F)CCC(=O)N4CCC(n5cc(-c6cnc7ccccc7n6)cn5)CC4)ccc3C2=O)C(=O)N1] | N-((2-(2,6-dioxopiperidin-3-yl)-1- oxoisoindolin-5-yl)methyl)-2,2-difluoro- 5-oxo-5-(4-(4-(quinoxalin-2-yl)-1H- pyrazol-1-yl)piperidin-1-yl)pentanamide |
| GS-534 | 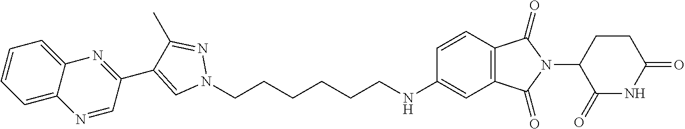 [SMILES: Cc1nn(CCCCCCNc2ccc3c(c2)C(=O)N(C2CCC(=O)NC2=O)C3=O)cc1-c1cnc2ccccc2n1] | 2-(2,6-dioxopiperidin-3-yl)-5-((6-(3- methyl-4-(quinoxalin-2-yl)-1H-pyrazol- 1-yl)hexyl)amino)isoindoline-1,3-dione |
| GS-535 | 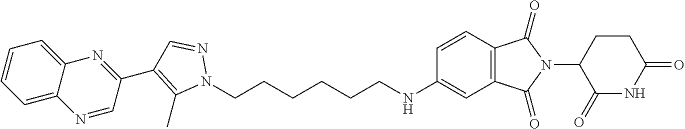 [SMILES: Cc1c(-c2cnc3ccccc3n2)cnn1CCCCCCNc1ccc2c(c1)C(=O)N(C1CCC(=O)NC1=O)C2=O] | 2-(2,6-dioxopiperidin-3-yl)-5-((6-(5- methyl-4-(quinoxalin-2-yl)-1H-pyrazol- 1-yl)hexyl)amino)isoindoline-1,3-dione |
| GS-536 | 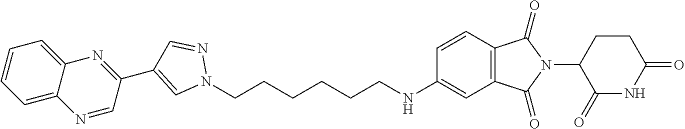 [SMILES: O=C1CCC(N2C(=O)c3ccc(NCCCCCCn4cc(-c5cnc6ccccc6n5)cn4)cc3C2=O)C(=O)N1] | 2-(2,6-dioxopiperidin-3-yl)-5-((6-(3- (quinoxalin-2-yl)-1H-pyrazol-1- yl)hexyl)amino)isoindoline-1,3-dione |
| GS-537 | 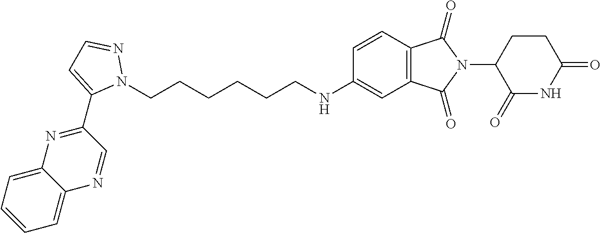 [SMILES: O=C1CCC(N2C(=O)c3ccc(NCCCCCCn4nccc4-c4cnc5ccccc5n4)cc3C2=O)C(=O)N1] | 2-(2,6-dioxopiperidin-3-yl)-5-((6-(5- (quinoxalin-2-yl)-1H-pyrazol-1- yl)hexyl)amino)isoindoline-1,3-dione |
| GS-538 | 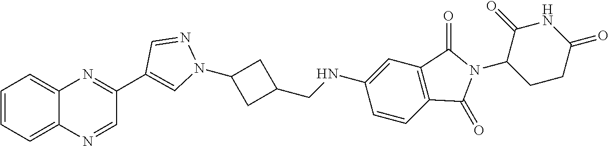 [SMILES: O=C1CCC(N2C(=O)c3ccc(NCC4CC(n5cc(-c6cnc7ccccc7n6)cn5)C4)cc3C2=O)C(=O)N1] | 2-(2,6-dioxopiperidin-3-yl)-5-(((3-(4- (quinoxalin-2-yl)-1H-pyrazol-1- yl)cyclobutyl)methyl)amino)isoindoline- 1,3-dione |
| GS-539 | 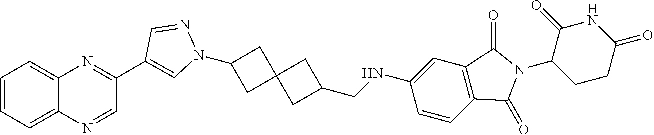 [SMILES: O=C1CCC(N2C(=O)c3ccc(NCC4CC5(C4)CC(n4cc(-c6cnc7ccccc7n6)cn4)C5)cc3C2=O)C(=O)N1] | 2-(2,6-dioxopiperidin-3-yl)-5-(((6-(4- (quinoxalin-2-yl)-1H-pyrazol-1- yl)spiro[3.3]heptan-2- yl)methyl)amino)isoindoline-1,3-dione |
| GS-540 | 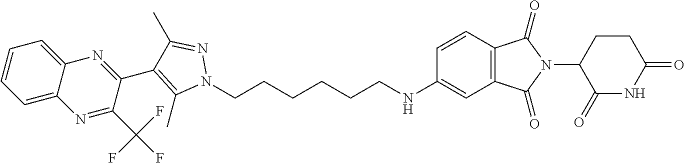 [SMILES: Cc1nn(CCCCCCNc2ccc3c(c2)C(=O)N(C2CCC(=O)NC2=O)C3=O)c(C)c1-c1nc2ccccc2nc1C(F)(F)F] | 5-((6-(3,5-dimethyl-4-(3- (trifluoromethyl)quinoxalin-2-yl)-1H- pyrazol-1-yl)hexyl)amino)-2-(2,6- dioxopiperidin-3-yl)isoindoline-1,3-dione |
| GS-541 | 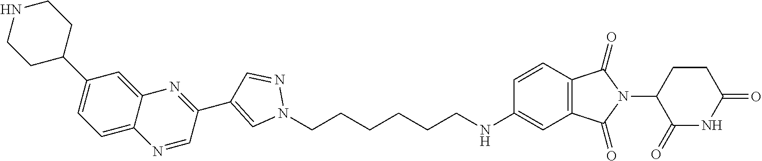 [SMILES: O=C1CCC(N2C(=O)c3ccc(NCCCCCCn4cc(-c5cnc6ccc(C7CCNCC7)cc6n5)cn4)cc3C2=O)C(=O)N1] | 2-(2,6-dioxopiperidin-3-yl)-5-((6-(4-(7- (piperidin-4-yl)quinoxalin-2-yl)-1H- pyrazol-1-yl)hexyl)amino)isoindoline- 1,3-dione |
| GS-542 | 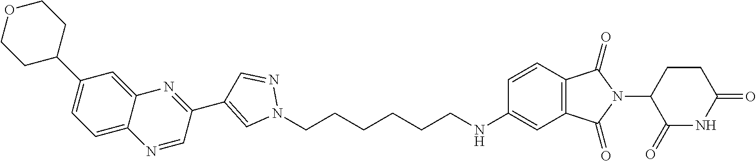 [SMILES: O=C1CCC(N2C(=O)c3ccc(NCCCCCCn4cc(-c5cnc6ccc(C7CCOCC7)cc6n5)cn4)cc3C2=O)C(=O)N1] | 2-(2,6-dioxopiperidin-3-yl)-5-((6-(4-(7- (tetrahydro-2H-pyran-4-yl)quinoxalin-2- yl)-1H-pyrazol-1- yl)hexyl)amino)isoindoline-1,3-dione |
| GS-543 | 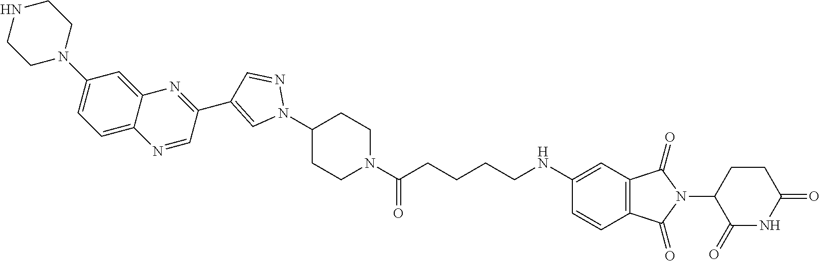 [SMILES: O=C1CCC(N2C(=O)c3ccc(NCCCCC(=O)N4CCC(n5cc(-c6cnc7ccc(N8CCNCC8)cc7n6)cn5)CC4)cc3C2=O)C(=O)N1] | 2-(2,6-dioxopiperidin-3-yl)-5-((5-oxo-5- (4-(4-(7-(piperazin-1-yl)quinoxalin-2-yl)- 1H-pyrazol-1-yl)piperidin-1- yl)pentyl)amino)isoindoline-1,3-dione |
| GS-544 | 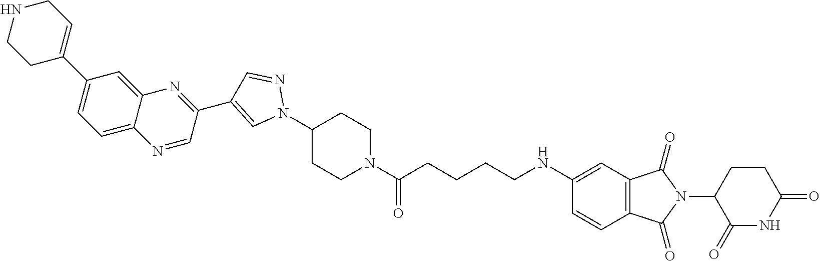 [SMILES: O=C1CCC(N2C(=O)c3ccc(NCCCCC(=O)N4CCC(n5cc(-c6cnc7ccc(C8=CCNCC8)cc7n6)cn5)CC4)cc3C2=O)C(=O)N1] | 2-(2,6-dioxopiperidin-3-yl)-5-((5-oxo-5- (4-(4-(7-(1,2,3,6-tetrahydropyridin-4- yl)quinoxalin-2-yl)-1H-pyrazol-1- yl)piperidin-1- yl)pentyl)amino)isoindoline-1,3-dione |
| GS-545 | 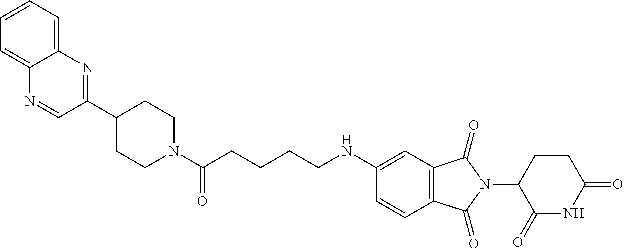 [SMILES: O=C1CCC(N2C(=O)c3ccc(NCCCCC(=O)N4CCC(c5cnc6ccccc6n5)CC4)cc3C2=O)C(=O)N1] | 2-(2,6-dioxopiperidin-3-yl)-5-((5-oxo-5- (4-(quinoxalin-2-yl)piperidin-1- yl)pentyl)amino)isoindoline-1,3-dione |
| GS-546 | 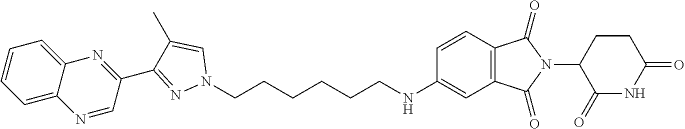 [SMILES: Cc1cn(CCCCCCNc2ccc3c(c2)C(=O)N(C2CCC(=O)NC2=O)C3=O)nc1-c1cnc2ccccc2n1] | 2-(2,6-dioxopiperidin-3-yl)-5-((6-(4- methyl-3-(quinoxalin-2-yl)-1H-pyrazol- 1-yl)hexyl)amino)isoindoline-1,3-dione |
| GS-547 | 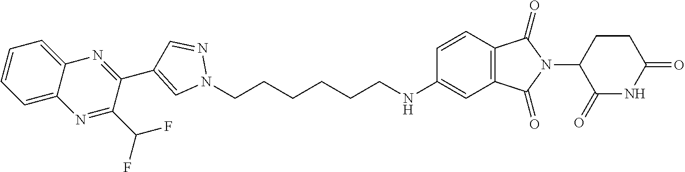 [SMILES: O=C1CCC(N2C(=O)c3ccc(NCCCCCCn4cc(-c5nc6ccccc6nc5C(F)F)cn4)cc3C2=O)C(=O)N1] | 5-((6-(4-(3-(difluoromethyl)quinoxalin-2- yl)-1H-pyrazol-1-yl)hexyl)amino)-2-(2,6- dioxopiperidin-3-yl)isoindoline-1,3-dione |
| GS-548 | 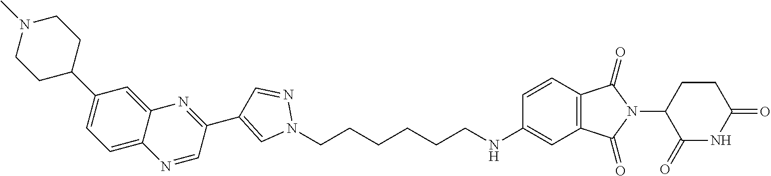 [SMILES: CN1CCC(c2ccc3ncc(-c4cnn(CCCCCCNc5ccc6c(c5)C(=O)N(C5CCC(=O)NC5=O)C6=O)c4)nc3c2)CC1] | 2-(2,6-dioxopiperidin-3-yl)-5-((6-(4-(7- (1-methylpiperidin-4-yl)quinoxalin-2-yl)- 1H-pyrazol-1- yl)hexyl)amino)isoindoline-1,3-dione |
| GS-549 | 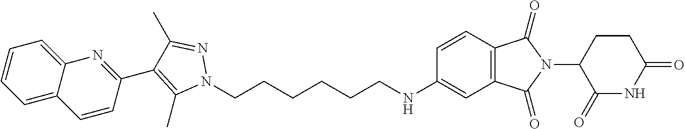 [SMILES: Cc1nn(CCCCCCNc2ccc3c(c2)C(=O)N(C2CCC(=O)NC2=O)C3=O)c(C)c1-c1ccc2ccccc2n1] | 5-((6-(3,5-dimethyl-4-(quinolin-2-yl)-1H- pyrazol-1-yl)hexyl)amino)-2-(2,6- dioxopiperidin-3-yl)isoindoline-1,3-dione |
| GS-550 | 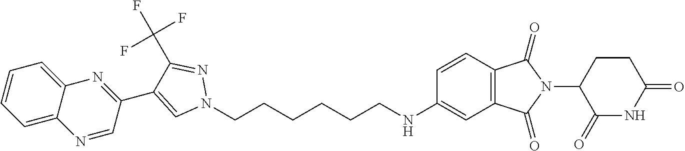 [SMILES: O=C1CCC(N2C(=O)c3ccc(NCCCCCCn4cc(-c5cnc6ccccc6n5)c(C(F)(F)F)n4)cc3C2=O)C(=O)N1] | 2-(2,6-dioxopiperidin-3-yl)-5-((6-(4- (quinoxalin-2-yl)-3-(trifluoromethyl)-1H- pyrazol-1-yl)hexyl)amino)isoindoline- 1,3-dione |
| GS-551 | 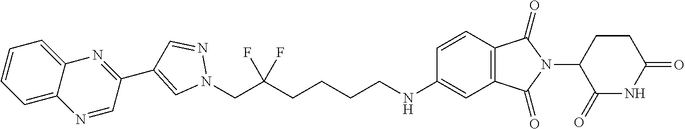 [SMILES: O=C1CCC(N2C(=O)c3ccc(NCCCCC(F)(F)Cn4cc(-c5cnc6ccccc6n5)cn4)cc3C2=O)C(=O)N1] | 5-((5,5-difluoro-6-(4-(quinoxalin-2-yl)- 1H-pyrazol-1-yl)hexyl)amino)-2-(2,6- dioxopiperidin-3-yl)isoindoline-1,3-dione |
| GS-552 | 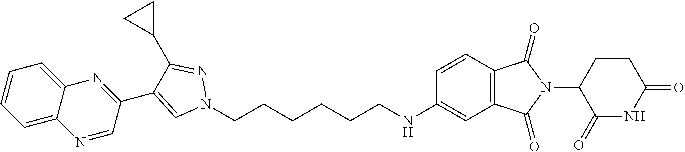 [SMILES: O=C1CCC(N2C(=O)c3ccc(NCCCCCCn4cc(-c5cnc6ccccc6n5)c(C5CC5)n4)cc3C2=O)C(=O)N1] | 5-((6-(3-cyclopropyl-4-(quinoxalin-2-yl)- 1H-pyrazol-1-yl)hexyl)amino)-2-(2,6- dioxopiperidin-3-yl)isoindoline-1,3-dione |
| GS-553 | 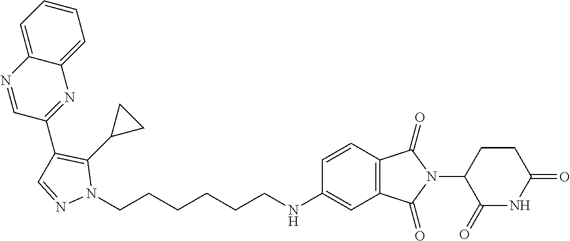 [SMILES: O=C1CCC(N2C(=O)c3ccc(NCCCCCCn4ncc(-c5cnc6ccccc6n5)c4C4CC4)cc3C2=O)C(=O)N1] | 5-((6-(5-cyclopropyl-4-(quinoxalin-2-yl)- 1H-pyrazol-1-yl)hexyl)amino)-2-(2,6- dioxopiperidin-3-yl)isoindoline-1,3-dione |
| GS-554 | 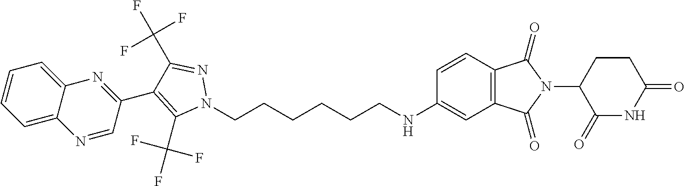 [SMILES: O=C1CCC(N2C(=O)c3ccc(NCCCCCCn4nc(C(F)(F)F)c(-c5cnc6ccccc6n5)c4C(F)(F)F)cc3C2=O)C(=O)N1] | 2-(2,6-dioxopiperidin-3-yl)-5-((6-(4- (quinoxalin-2-yl)-3,5- bis(trifluoromethyl)-1H-pyrazol-1- yl)hexyl)amino)isoindoline-1,3-dione |
| GS-555 | 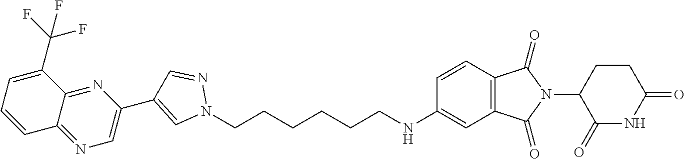 [SMILES: O=C1CCC(N2C(=O)c3ccc(NCCCCCCn4cc(-c5cnc6cccc(C(F)(F)F)c6n5)cn4)cc3C2=O)C(=O)N1] | 2-(2,6-dioxopiperidin-3-yl)-5-((6-(4-(8- (trifluoromethyl)quinoxalin-2-yl)-1H- pyrazol-1-yl)hexyl)amino)isoindoline- 1,3-dione |
| GS-556 | 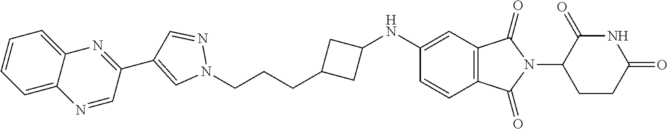 [SMILES: O=C1CCC(N2C(=O)c3ccc(NC4CC(CCCn5cc(-c6cnc7ccccc7n6)cn5)C4)cc3C2=O)C(=O)N1] | 2-(2,6-dioxopiperidin-3-yl)-5-((3-(3-(4- (quinoxalin-2-yl)-1H-pyrazol-1- yl)propyl)cyclobutyl)amino)isoindoline- 1,3-dione |
| GS-557 | 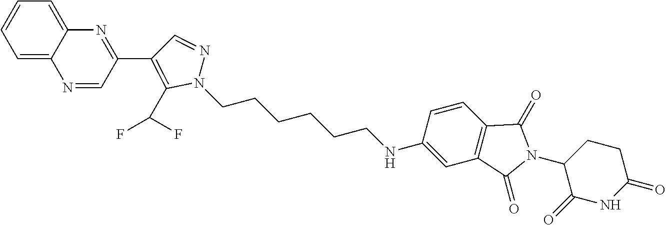 [SMILES: O=C1CCC(N2C(=O)c3ccc(NCCCCCCn4ncc(-c5cnc6ccccc6n5)c4C(F)F)cc3C2=O)C(=O)N1] | 5-((6-(5-(difluoromethyl)-4-(quinoxalin- 2-yl)-1H-pyrazol-1-yl)hexyl)amino)-2- (2,6-dioxopiperidin-3-yl)isoindoline-1,3- dione |
| GS-558 | 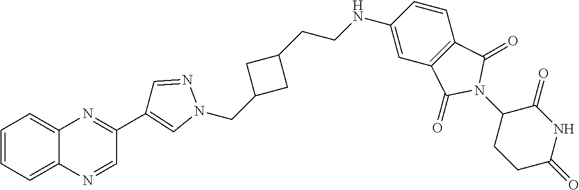 [SMILES: O=C1CCC(N2C(=O)c3ccc(NCCC4CC(Cn5cc(-c6cnc7ccccc7n6)cn5)C4)cc3C2=O)C(=O)N1] | 2-(2,6-dioxopiperidin-3-yl)-5-((2-(3-((4- (quinoxalin-2-yl)-1H-pyrazol-1- yl)methyl)cyclobutyl)ethyl)amino)iso- indoline-1,3-dione |
| GS-559 | 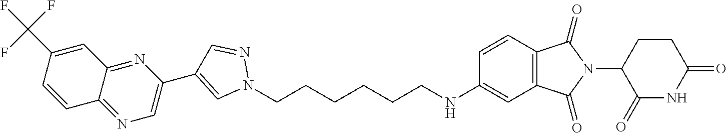 [SMILES: O=C1CCC(N2C(=O)c3ccc(NCCCCCCn4cc(-c5cnc6ccc(C(F)(F)F)cc6n5)cn4)cc3C2=O)C(=O)N1] | 2-(2,6-dioxopiperidin-3-yl)-5-((6-(4-(7- (trifluoromethyl)quinoxalin-2-yl)-1H- pyrazol-1-yl)hexyl)amino)isoindoline- 1,3-dione |
| GS-560 | 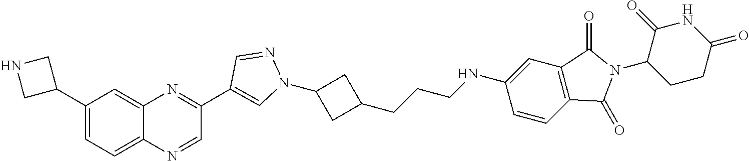 [SMILES: O=C1CCC(N2C(=O)c3ccc(NCCCC4CC(n5cc(-c6cnc7ccc(C8CNC8)cc7n6)cn5)C4)cc3C2=O)C(=O)N1] | 5-((3-(3-(4-(7-(azetidin-3-yl)quinoxalin- 2-yl)-1H-pyrazol-1- yl)cyclobutyl)propyl)amino)-2-(2,6- dioxopiperidin-3-yl)isoindoline-1,3-dione |
| GS-561 | 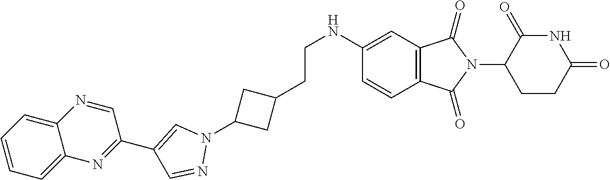 [SMILES: O=C1CCC(N2C(=O)c3ccc(NCCC4CC(n5cc(-c6cnc7ccccc7n6)cn5)C4)cc3C2=O)C(=O)N1] | 2-(2,6-dioxopiperidin-3-yl)-5-((2-(3-(4- (quinoxalin-2-yl)-1H-pyrazol-1- yl)cyclobutyl)ethyl)amino)isoindoline- 1,3-dione |
| GS-562 | 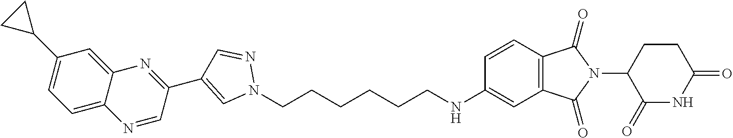 [SMILES: O=C1CCC(N2C(=O)c3ccc(NCCCCCCn4cc(-c5cnc6ccc(C7CC7)cc6n5)cn4)cc3C2=O)C(=O)N1] | 5-((6-(4-(7-cyclopropylquinoxalin-2-yl)- 1H-pyrazol-1-yl)hexyl)amino)-2-(2,6- dioxopiperidin-3-yl)isoindoline-1,3-dione |
| GS-563 | 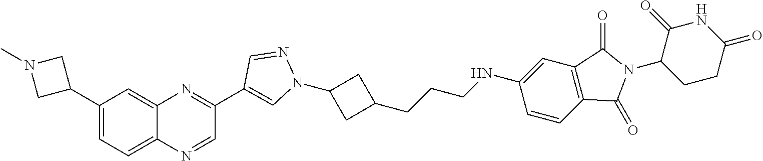 [SMILES: CN1CC(c2ccc3ncc(-c4cnn(C5CC(CCCNc6ccc7c(c6)C(=O)N(C6CCC(=O)NC6=O)C7=O)C5)c4)nc3c2)C1] | 2-(2,6-dioxopiperidin-3-yl)-5-((3-(3-(4- (7-(1-methylazetidin-3-yl)quinoxalin-2- yl)-1H-pyrazol-1- yl)cyclobutyl)propyl)amino)isoindoline- 1,3-dione |
| GS-564 | 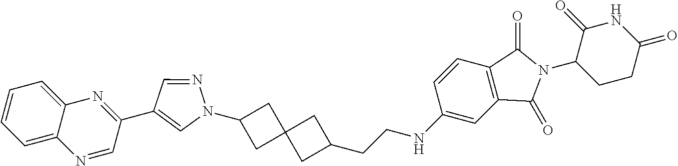 [SMILES: O=C1CCC(N2C(=O)c3ccc(NCCC4CC5(C4)CC(n4cc(-c6cnc7ccccc7n6)cn4)C5)cc3C2=O)C(=O)N1] | 2-(2,6-dioxopiperidin-3-yl)-5-((2-(6-(4- (quinoxalin-2-yl)-1H-pyrazol-1- yl)spiro[3.3]heptan-2- yl)ethyl)amino)isoindoline-1,3-dione |
| GS-565 | 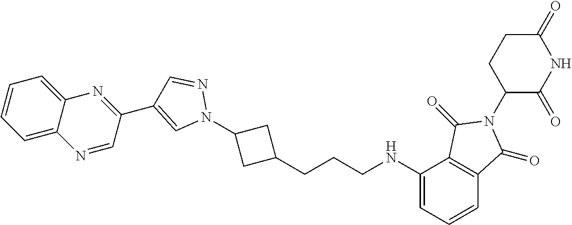 [SMILES: O=C1CCC(N2C(=O)c3cccc(NCCCC4CC(n5cc(-c6cnc7ccccc7n6)cn5)C4)c3C2=O)C(=O)N1] | 2-(2,6-dioxopiperidin-3-yl)-4-((3-(3-(4- (quinoxalin-2-yl)-1H-pyrazol-1- yl)cyclobutyl)propyl)amino)isoindoline- 1,3-dione |
| GS-566 | 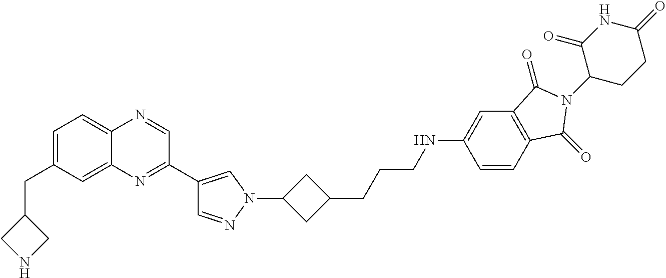 [SMILES: O=C1CCC(N2C(=O)c3ccc(NCCCC4CC(n5cc(-c6cnc7ccc(CC8CNC8)cc7n6)cn5)C4)cc3C2=O)C(=O)N1] | 5-((3-(3-(4-(7-(azetidin-3- ylmethyl)quinoxalin-2-yl)-1H-pyrazol-1- yl)cyclobutyl)propyl)amino)-2-(2,6- dioxopiperidin-3-yl)isoindoline-1,3-dione |
| GS-567 | 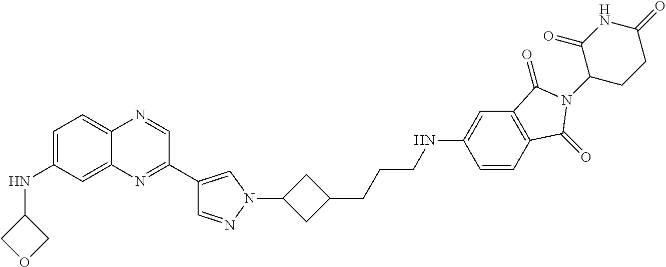 [SMILES: O=C1CCC(N2C(=O)c3ccc(NCCCC4CC(n5cc(-c6cnc7ccc(NC8COC8)cc7n6)cn5)C4)cc3C2=O)C(=O)N1] | 2-(2,6-dioxopiperidin-3-yl)-5-((3-(3-(4- (7-(oxetan-3-ylamino)quinoxalin-2-yl)- 1H-pyrazol-1- yl)cyclobutyl)propyl)amino)isoindoline- 1,3-dione |
| GS-568 | 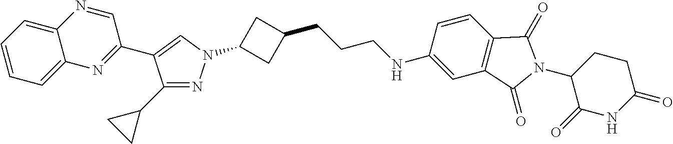 [SMILES: O=C1CCC(N2C(=O)c3ccc(NCCC[C@H]4C[C@H](n5cc(-c6cnc7ccccc7n6)c(C6CC6)n5)C4)cc3C2=O)C(=O)N1] | 5-((3-(trans-3-(3-cyclopropyl-4- (quinoxalin-2-yl)-1H-pyrazol-1- yl)cyclobutyl)propyl)amino)-2-(2,6- dioxopiperidin-3-yl)isoindoline-1,3-dione |
| GS-569 | 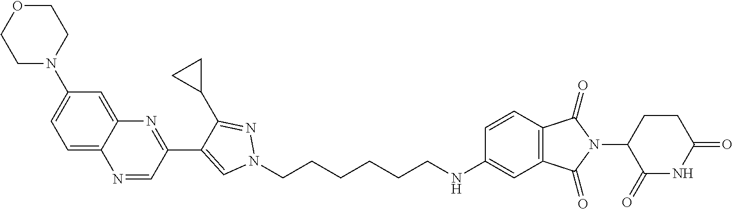 [SMILES: O=C1CCC(N2C(=O)c3ccc(NCCCCCCn4cc(-c5cnc6ccc(N7CCOCC7)cc6n5)c(C5CC5)n4)cc3C2=O)C(=O)N1] | 5-((6-(3-cyclopropyl-4-(7- morpholinoquinoxalin-2-yl)-1H-pyrazol- 1-yl)hexyl)amino)-2-(2,6-dioxopiperidin- 3-yl)isoindoline-1,3-dione |
| GS-570 | 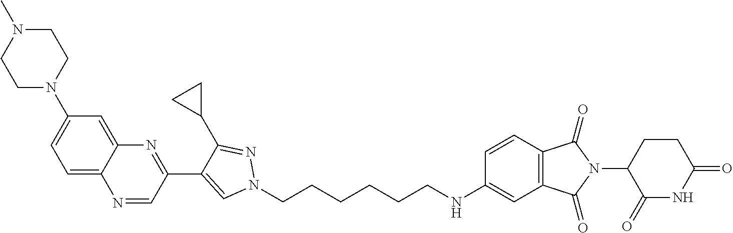 [SMILES: CN1CCN(c2ccc3ncc(-c4cn(CCCCCCNc5ccc6c(c5)C(=O)N(C5CCC(=O)NC5=O)C6=O)nc4C4CC4)nc3c2)CC1] | 5-((6-(3-cyclopropyl-4-(7-(4- methylpiperazin-1-yl)quinoxalin-2-yl)- 1H-pyrazol-1-yl)hexyl)amino)-2-(2,6- dioxopiperidin-3-yl)isoindoline-1,3-dione |
| GS-571 | 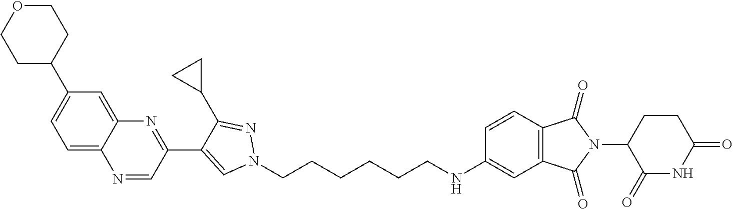 [SMILES: O=C1CCC(N2C(=O)c3ccc(NCCCCCCn4cc(-c5cnc6ccc(C7CCOCC7)cc6n5)c(C5CC5)n4)cc3C2=O)C(=O)N1] | 5-((6-(3-cyclopropyl-4-(7-(tetrahydro- 2H-pyran-4-yl)quinoxalin-2-yl)-1H- pyrazol-1-yl)hexyl)amino)-2-(2,6- dioxopiperidin-3-yl)isoindoline-1,3-dione |
| GS-572 | 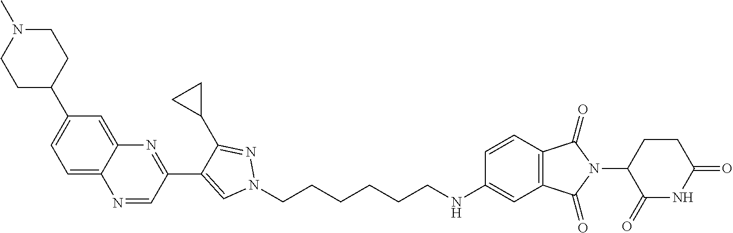 [SMILES: CN1CCC(c2ccc3ncc(-c4cn(CCCCCCNc5ccc6c(c5)C(=O)N(C5CCC(=O)NC5=O)C6=O)nc4C4CC4)nc3c2)CC1] | 5-((6-(3-cyclopropyl-4-(7-(1- methylpiperidin-4-yl)quinoxalin-2-yl)- 1H-pyrazol-1-yl)hexyl)amino)-2-(2,6- dioxopiperidin-3-yl)isoindoline-1,3-dione |
| GS-573 | 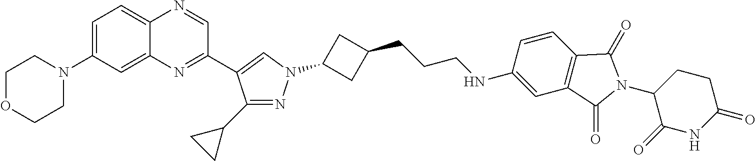 [SMILES: O=C1CCC(N2C(=O)c3ccc(NCCC[C@H]4C[C@H](n5cc(-c6cnc7ccc(N8CCOCC8)cc7n6)c(C6CC6)n5)C4)cc3C2=O)C(=O)N1] | 5-((3-(trans-3-(3-cyclopropyl-4-(7- morpholinoquinoxalin-2-yl)-1H-pyrazol- 1-yl)cyclobutyl)propyl)amino)-2-(2,6- dioxopiperidin-3-yl)isoindoline-1,3-dione |
| GS-574 | 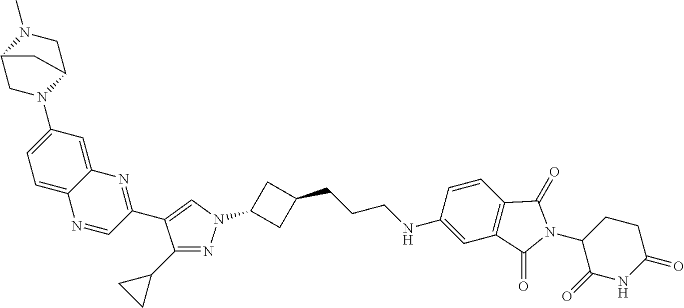 [SMILES: CN1C[C@H]2C[C@@H]1CN2c1ccc2ncc(-c3cn([C@H]4C[C@H](CCCNc5ccc6c(c5)C(=O)N(C5CCC(=O)NC5=O)C6=O)C4)nc3C3CC3)nc2c1] | 5-((3-(trans-3-(3-cyclopropyl-4-(7- ((1R,4R)-5-methyl-2,5- diazabicyclo[2.2.1]heptan-2- yl)quinoxalin-2-yl)-1H-pyrazol-1- yl)cyclobutyl)propyl)amino)-2-(2,6- dioxopiperidin-3-yl)isoindoline-1,3-dione |
| GS-575 | 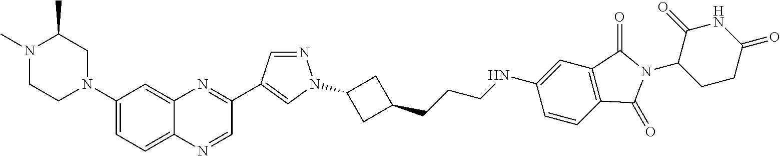 [SMILES: C[C@H]1CN(c2ccc3ncc(-c4cnn([C@H]5C[C@H](CCCNc6ccc7c(c6)C(=O)N(C6CCC(=O)NC6=O)C7=O)C5)c4)nc3c2)CCN1C] | 5-((3-(trans-3-(4-(7-((S)-3,4- dimethylpiperazin-1-yl)quinoxalin-2-yl)- 1H-pyrazol-1- yl)cyclobutyl)propyl)amino)-2-(2,6- dioxopiperidin-3-yl)isoindoline-1,3-dione |
| GS-576 | 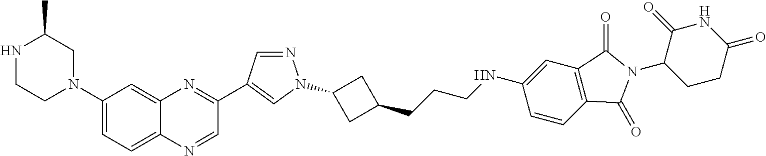 [SMILES: C[C@H]1CN(c2ccc3ncc(-c4cnn([C@H]5C[C@H](CCCNc6ccc7c(c6)C(=O)N(C6CCC(=O)NC6=O)C7=O)C5)c4)nc3c2)CCN1] | 2-(2,6-dioxopiperidin-3-yl)-5-((3-(trans- 3-(4-(7-((S)-3-methylpiperazin-1- yl)quinoxalin-2-yl)-1H-pyrazol-1- yl)cyclobutyl)propyl)amino)isoindoline- 1,3-dione |
| GS-577 | 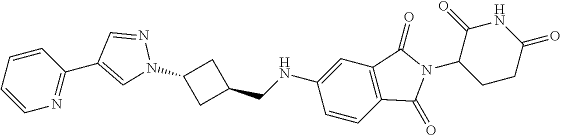 [SMILES: O=C1CCC(N2C(=O)c3ccc(NC[C@H]4C[C@H](n5cc(-c6ccccn6)cn5)C4)cc3C2=O)C(=O)N1] | 2-(2,6-dioxopiperidin-3-yl)-5-(((trans-3- (4-(pyridin-2-yl)-1H-pyrazol-1- yl)cyclobutyl)methyl)amino)isoindoline- 1,3-dione |
| GS-578 | 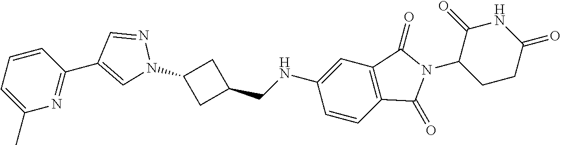 [SMILES: Cc1cccc(-c2cnn([C@H]3C[C@H](CNc4ccc5c(c4)C(=O)N(C4CCC(=O)NC4=O)C5=O)C3)c2)n1] | 2-(2,6-dioxopiperidin-3-yl)-5-(((trans-3- (4-(6-methylpyridin-2-yl)-1H-pyrazol-1- yl)cyclobutyl)methyl)amino)isoindoline- 1,3-dione |
| GS-579 | 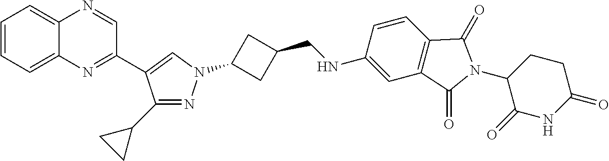 [SMILES: O=C1CCC(N2C(=O)c3ccc(NC[C@H]4C[C@H](n5cc(-c6cnc7ccccc7n6)c(C6CC6)n5)C4)cc3C2=O)C(=O)N1] | 5-(((trans-3-(3-cyclopropyl-4- (quinoxalin-2-yl)-1H-pyrazol-1- yl)cyclobutyl)methyl)amino)-2-(2,6- dioxopiperidin-3-yl)isoindoline-1,3-dione |
| GS-580 | 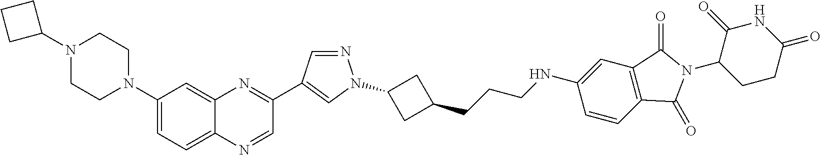 [SMILES: O=C1CCC(N2C(=O)c3ccc(NCCC[C@H]4C[C@H](n5cc(-c6cnc7ccc(N8CCN(C9CCC9)CC8)cc7n6)cn5)C4)cc3C2=O)C(=O)N1] | 5-((3-(trans-3-(4-(7-(4- cyclobutylpiperazin-1-yl)quinoxalin-2- yl)-1H-pyrazol-1- yl)cyclobutyl)propyl)amino)-2-(2,6- dioxopiperidin-3-yl)isoindoline-1,3-dione |
| GS-581 | 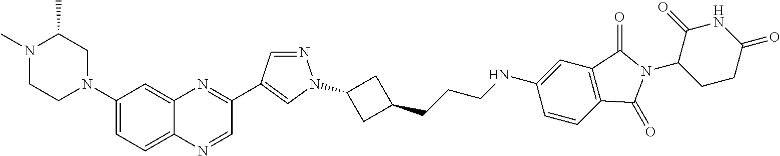 [SMILES: C[C@@H]1CN(c2ccc3ncc(-c4cnn([C@H]5C[C@H](CCCNc6ccc7c(c6)C(=O)N(C6CCC(=O)NC6=O)C7=O)C5)c4)nc3c2)CCN1C] | 5-((3-(trans-3-(4-(7-((R)-3,4- dimethylpiperazin-1-yl)quinoxalin-2-yl)- 1H-pyrazol-1- yl)cyclobutyl)propyl)amino)-2-(2,6- dioxopiperidin-3-yl)isoindoline-1,3-dione |
| GS-582 | 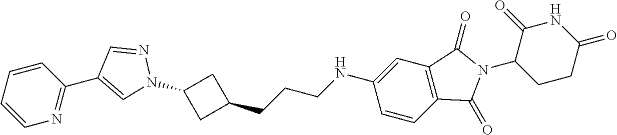 [SMILES: O=C1CCC(N2C(=O)c3ccc(NCCC[C@H]4C[C@H](n5cc(-c6ccccn6)cn5)C4)cc3C2=O)C(=O)N1] | 2-(2,6-dioxopiperidin-3-yl)-5-((3-(trans- 3-(4-(pyridin-2-yl)-1H-pyrazol-1- yl)cyclobutyl)propyl)amino)isoindoline- 1,3-dione |
| GS-583 | 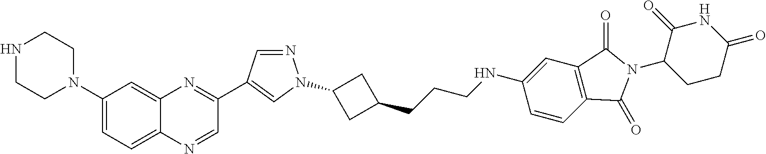 [SMILES: O=C1CCC(N2C(=O)c3ccc(NCCC[C@H]4C[C@H](n5cc(-c6cnc7ccc(N8CCNCC8)cc7n6)cn5)C4)cc3C2=O)C(=O)N1] | 2-(2,6-dioxopiperidin-3-yl)-5-((3-(trans- 3-(4-(7-(piperazin-1-yl)quinoxalin-2-yl)- 1H-pyrazol-1- yl)cyclobutyl)propyl)amino)isoindoline- 1,3-dione |
| GS-584 | 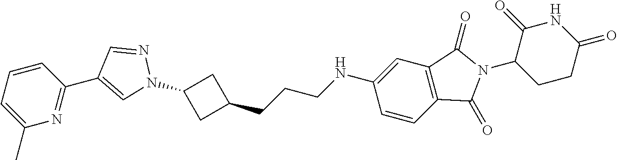 [SMILES: Cc1cccc(-c2cnn([C@H]3C[C@H](CCCNc4ccc5c(c4)C(=O)N(C4CCC(=O)NC4=O)C5=O)C3)c2)n1] | 2-(2,6-dioxopiperidin-3-yl)-5-((3-(trans- 3-(4-(6-methylpyridin-2-yl)-1H-pyrazol- 1- yl)cyclobutyl)propyl)amino)isoindoline- 1,3-dione |
| GS-585 | 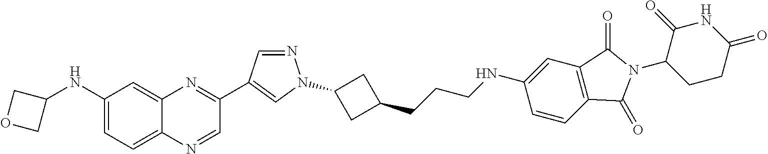 [SMILES: O=C1CCC(N2C(=O)c3ccc(NCCC[C@H]4C[C@H](n5cc(-c6cnc7ccc(NC8COC8)cc7n6)cn5)C4)cc3C2=O)C(=O)N1] | 2-(2,6-dioxopiperidin-3-yl)-5-((3-(trans- 3-(4-(7-(oxetan-3-ylamino)quinoxalin-2- yl)-1H-pyrazol-1- yl)cyclobutyl)propyl)amino)isoindoline- 1,3-dione |
| GS-586 | 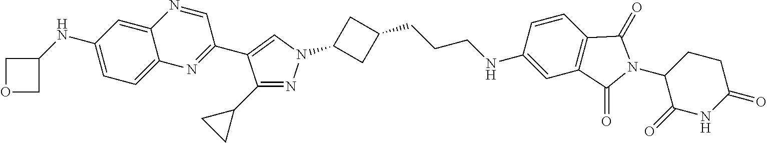 [SMILES: O=C1CCC(N2C(=O)c3ccc(NCCC[C@H]4C[C@@H](n5cc(-c6cnc7cc(NC8COC8)ccc7n6)c(C6CC6)n5)C4)cc3C2=O)C(=O)N1] | 5-((3-(cis-3-(3-cyclopropyl-4-(6-(oxetan- 3-ylamino)quinoxalin-2-yl)-1H-pyrazol- 1-yl)cyclobutyl)propyl)amino)-2-(2,6- dioxopiperidin-3-yl)isoindoline-1,3-dione |
| GS-587 | 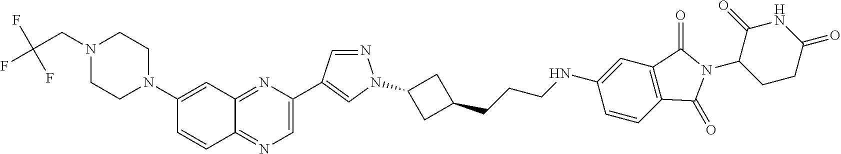 [SMILES: O=C1CCC(N2C(=O)c3ccc(NCCC[C@H]4C[C@H](n5cc(-c6cnc7ccc(N8CCN(CC(F)(F)F)CC8)cc7n6)cn5)C4)cc3C2=O)C(=O)N1] | 2-(2,6-dioxopiperidin-3-yl)-5-((3-(trans- 3-(4-(7-(4-(2,2,2-trifluoroethyl)piperazin- 1-yl)quinoxalin-2-yl)-1H-pyrazol-1- yl)cyclobutyl)propyl)amino)isoindoline- 1,3-dione |
| GS-588 | 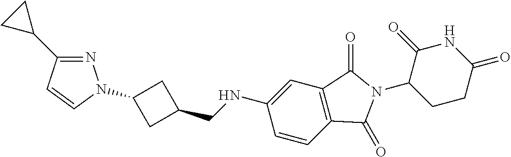 [SMILES: O=C1CCC(N2C(=O)c3ccc(NC[C@H]4C[C@H](n5ccc(C6CC6)n5)C4)cc3C2=O)C(=O)N1] | 5-(((trans-3-(3-cyclopropyl-1H-pyrazol- 1-yl)cyclobutyl)methyl)amino)-2-(2,6- dioxopiperidin-3-yl)isoindoline-1,3-dione |
| GS-589 | 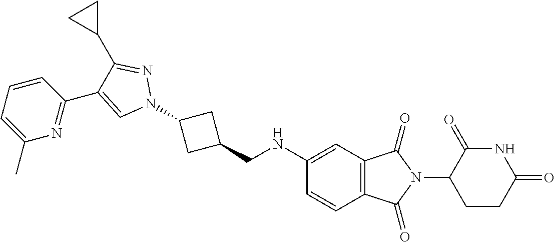 [SMILES: Cc1cccc(-c2cn([C@H]3C[C@H](CNc4ccc5c(c4)C(=O)N(C4CCC(=O)NC4=O)C5=O)C3)nc2C2CC2)n1] | 5-(((trans-3-(3-cyclopropyl-4-(6- methylpyridin-2-yl)-1H-pyrazol-1- yl)cyclobutyl)methyl)amino)-2-(2,6- dioxopiperidin-3-yl)isoindoline-1,3-dione |
| GS-590 | 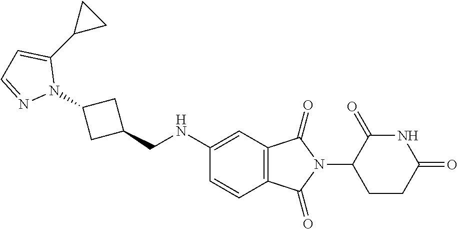 [SMILES: O=C1CCC(N2C(=O)c3ccc(NC[C@H]4C[C@H](n5nccc5C5CC5)C4)cc3C2=O)C(=O)N1] | 5-(((trans-3-(5-cyclopropyl-1H-pyrazol- 1-yl)cyclobutyl)methyl)amino)-2-(2,6- dioxopiperidin-3-yl)isoindoline-1,3-dione |
| GS-591 | 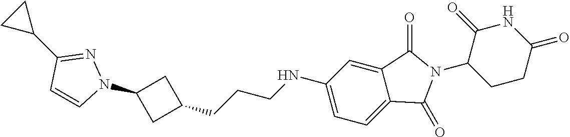 [SMILES: O=C1CCC(N2C(=O)c3ccc(NCCC[C@H]4C[C@H](n5ccc(C6CC6)n5)C4)cc3C2=O)C(=O)N1] | 5-((3-(trans-3-(3-cyclopropyl-1H- pyrazol-1-yl)cyclobutyl)propyl)amino)-2- (2,6-dioxopiperidin-3-yl)isoindoline-1,3- dione |
| GS-592 | 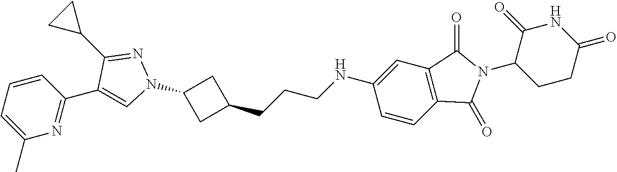 [SMILES: Cc1cccc(-c2cn([C@H]3C[C@H](CCCNc4ccc5c(c4)C(=O)N(C4CCC(=O)NC4=O)C5=O)C3)nc2C2CC2)n1] | 5-((3-(trans-3-(3-cyclopropyl-4-(6- methylpyridin-2-yl)-1H-pyrazol-1- yl)cyclobutyl)propyl)amino)-2-(2,6- dioxopiperidin-3-yl)isoindoline-1,3-dione |
| GS-593 | 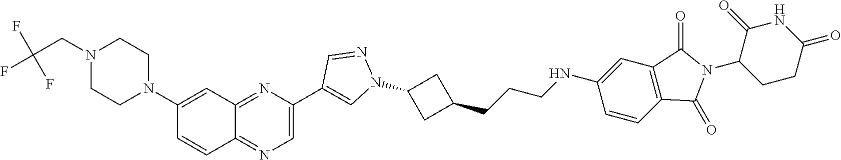 [SMILES: O=C1CCC(N2C(=O)c3ccc(NCCC[C@H]4C[C@H](n5cc(-c6cnc7ccc(N8CCN(CC(F)(F)F)CC8)cc7n6)cn5)C4)cc3C2=O)C(=O)N1] | 2-(2,6-dioxopiperidin-3-yl)-5-((3-(trans- 3-(4-(7-(4-(2,2,2-trifluoroethyl)piperazin- 1-yl)quinoxalin-2-yl)-1H-pyrazol-1- yl)cyclobutyl)propyl)amino)isoindoline- 1,3-dione |
| GS-594 | 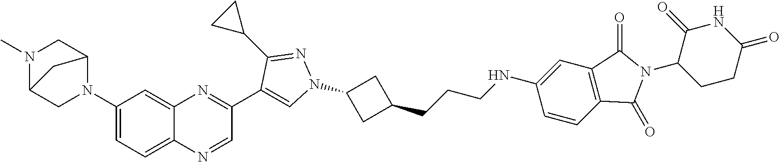 [SMILES: CN1CC2CC1CN2c1ccc2ncc(-c3cn([C@H]4C[C@H](CCCNc5ccc6c(c5)C(=O)N(C5CCC(=O)NC5=O)C6=O)C4)nc3C3CC3)nc2c1] | 5-((3-(trans-3-(3-cyclopropyl-4-(7- ((1S,4S)-5-methyl-2,5- diazabicyclo[2.2.1]heptan-2- yl)quinoxalin-2-yl)-1H-pyrazol-1- yl)cyclobutyl)propyl)amino)-2-(2,6- dioxopiperidin-3-yl)isoindoline-1,3-dione |
| GS-595 | 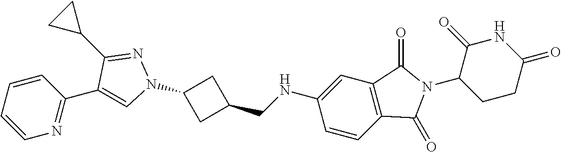 [SMILES: O=C1CCC(N2C(=O)c3ccc(NC[C@H]4C[C@H](n5cc(-c6ccccn6)c(C6CC6)n5)C4)cc3C2=O)C(=O)N1] | 5-(((trans-3-(3-cyclopropyl-4-(pyridin-2- yl)-1H-pyrazol-1- yl)cyclobutyl)methyl)amino)-2-(2,6- dioxopiperidin-3-yl)isoindoline-1,3-dione |
| GS-596 | 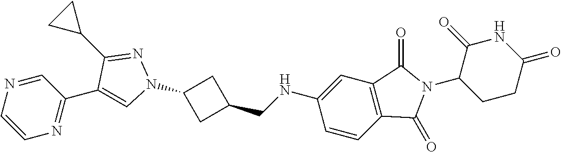 [SMILES: O=C1CCC(N2C(=O)c3ccc(NC[C@H]4C[C@H](n5cc(-c6cnccn6)c(C6CC6)n5)C4)cc3C2=O)C(=O)N1] | 5-(((trans-3-(3-cyclopropyl-4-(pyrazin-2- yl)-1H-pyrazol-1- yl)cyclobutyl)methyl)amino)-2-(2,6- dioxopiperidin-3-yl)isoindoline-1,3-dione |
| GS-597 | 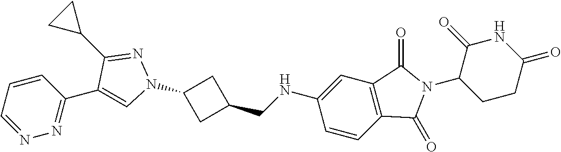 [SMILES: O=C1CCC(N2C(=O)c3ccc(NC[C@H]4C[C@H](n5cc(-c6cccnn6)c(C6CC6)n5)C4)cc3C2=O)C(=O)N1] | 5-(((trans-3-(3-cyclopropyl-4-(pyridazin- 3-yl)-1H-pyrazol-1- yl)cyclobutyl)methyl)amino)-2-(2,6- dioxopiperidin-3-yl)isoindoline-1,3-dione |
| GS-598 | 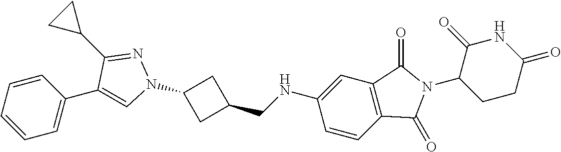 [SMILES: O=C1CCC(N2C(=O)c3ccc(NC[C@H]4C[C@H](n5cc(-c6ccccc6)c(C6CC6)n5)C4)cc3C2=O)C(=O)N1] | 5-(((trans-3-(3-cyclopropyl-4-phenyl-1H- pyrazol-1-yl)cyclobutyl)methyl)amino)- 2-(2,6-dioxopiperidin-3-yl)isoindoline- 1,3-dione |
| GS-599 | 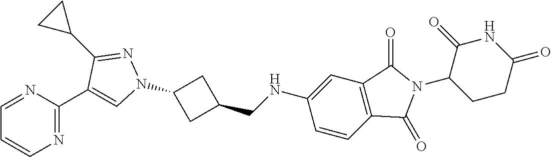 [SMILES: O=C1CCC(N2C(=O)c3ccc(NC[C@H]4C[C@H](n5cc(-c6ncccn6)c(C6CC6)n5)C4)cc3C2=O)C(=O)N1] | 5-(((trans-3-(3-cyclopropyl-4-(pyrimidin- 2-yl)-1H-pyrazol-1- yl)cyclobutyl)methyl)amino)-2-(2,6- dioxopiperidin-3-yl)isoindoline-1,3-dione |
| GS-600 | 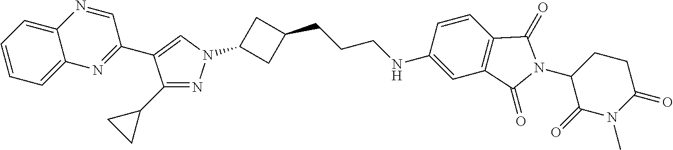 [SMILES: CN1C(=O)CCC(N2C(=O)c3ccc(NCCC[C@H]4C[C@H](n5cc(-c6cnc7ccccc7n6)c(C6CC6)n5)C4)cc3C2=O)C1=O] | 5-((3-(trans-3-(3-cyclopropyl-4- (quinoxalin-2-yl)-1H-pyrazol-1- yl)cyclobutyl)propyl)amino)-2-(1- methyl-2,6-dioxopiperidin-3- yl)isoindoline-1,3-dione |
| GS-601 | 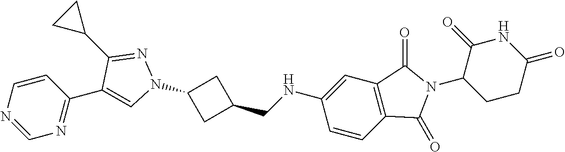 [SMILES: O=C1CCC(N2C(=O)c3ccc(NC[C@H]4C[C@H](n5cc(-c6ccncn6)c(C6CC6)n5)C4)cc3C2=O)C(=O)N1] | 5-(((trans-3-(3-cyclopropyl-4-(pyrimidin- 4-yl)-1H-pyrazol-1- yl)cyclobutyl)methyl)amino)-2-(2,6- dioxopiperidin-3-yl)isoindoline-1,3-dione |
| GS-602 | 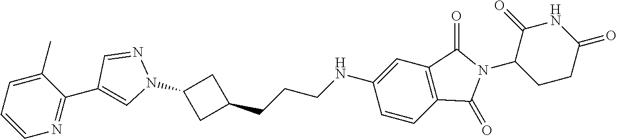 [SMILES: Cc1cccnc1-c1cnn([C@H]2C[C@H](CCCNc3ccc4c(c3)C(=O)N(C3CCC(=O)NC3=O)C4=O)C2)c1] | 2-(2,6-dioxopiperidin-3-yl)-5-((3-(trans- 3-(4-(3-methylpyridin-2-yl)-1H-pyrazol- 1- yl)cyclobutyl)propyl)amino)isoindoline- 1,3-dione |
| GS-603 | 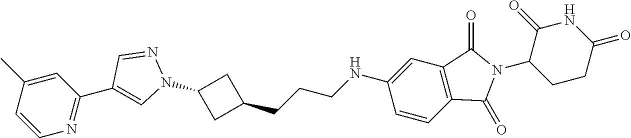 [SMILES: Cc1ccnc(-c2cnn([C@H]3C[C@H](CCCNc4ccc5c(c4)C(=O)N(C4CCC(=O)NC4=O)C5=O)C3)c2)c1] | 2-(2,6-dioxopiperidin-3-yl)-5-((3-(trans- 3-(4-(4-methylpyridin-2-yl)-1H-pyrazol- 1- yl)cyclobutyl)propyl)amino)isoindoline- 1,3-dione |
| GS-604 | 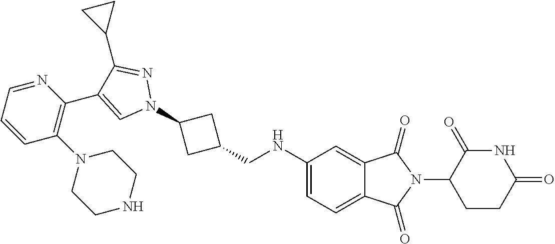 [SMILES: O=C1CCC(N2C(=O)c3ccc(NC[C@H]4C[C@H](n5cc(-c6ncccc6N6CCNCC6)c(C6CC6)n5)C4)cc3C2=O)C(=O)N1] | 5-(((trans-3-(3-cyclopropyl-4-(3- (piperazin-1-yl)pyridin-2-yl)-1H-pyrazol- 1-yl)cyclobutyl)methyl)amino)-2-(2,6- dioxopiperidin-3-yl)isoindoline-1,3-dione |
| GS-605 | 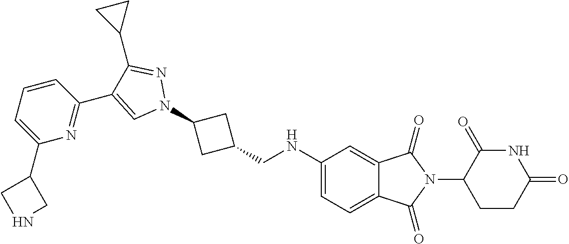 [SMILES: O=C1CCC(N2C(=O)c3ccc(NC[C@H]4C[C@H](n5cc(-c6cccc(C7CNC7)n6)c(C6CC6)n5)C4)cc3C2=O)C(=O)N1] | 5-(((trans-3-(4-(6-(azetidin-3-yl)pyridin- 2-yl)-3-cyclopropyl-1H-pyrazol-1- yl)cyclobutyl)methyl)amino)-2-(2,6- dioxopiperidin-3-yl)isoindoline-1,3-dione |
| GS-606 | 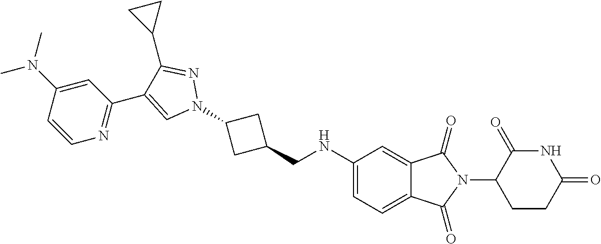 [SMILES: CN(C)c1ccnc(-c2cn([C@H]3C[C@H](CNc4ccc5c(c4)C(=O)N(C4CCC(=O)NC4=O)C5=O)C3)nc2C2CC2)c1] | 5-(((trans-3-(3-cyclopropyl-4-(4- (dimethylamino)pyridin-2-yl)-1H- pyrazol-1-yl)cyclobutyl)methyl)amino)- 2-(2,6-dioxopiperidin-3-yl)isoindoline- 1,3-dione |
| GS-607 | 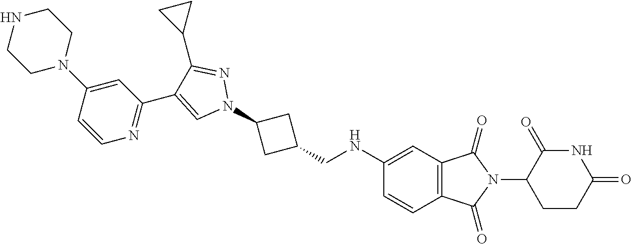 [SMILES: O=C1CCC(N2C(=O)c3ccc(NC[C@H]4C[C@H](n5cc(-c6cc(N7CCNCC7)ccn6)c(C6CC6)n5)C4)cc3C2=O)C(=O)N1] | 5-(((trans-3-(3-cyclopropyl-4-(4- (piperazin-1-yl)pyridin-2-yl)-1H-pyrazol- 1-yl)cyclobutyl)methyl)amino)-2-(2,6- dioxopiperidin-3-yl)isoindoline-1,3-dione |
| GS-608 | 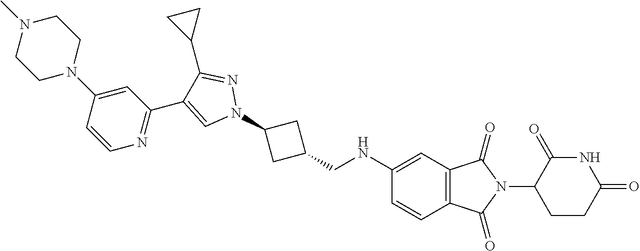 [SMILES: CN1CCN(c2ccnc(-c3cn([C@H]4C[C@H](CNc5ccc6c(c5)C(=O)N(C5CCC(=O)NC5=O)C6=O)C4)nc3C3CC3)c2)CC1] | 5-(((trans-3-(3-cyclopropyl-4-(4-(4- methylpiperazin-1-yl)pyridin-2-yl)-1H- pyrazol-1-yl)cyclobutyl)methyl)amino)- 2-(2,6-dioxopiperidin-3-yl)isoindoline- 1,3-dione |
| GS-609 | 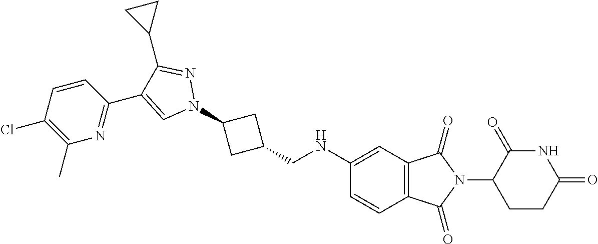 [SMILES: Cc1nc(-c2cn([C@H]3C[C@H](CNc4ccc5c(c4)C(=O)N(C4CCC(=O)NC4=O)C5=O)C3)nc2C2CC2)ccc1Cl] | 5-(((trans-3-(4-(5-chloro-6- methylpyridin-2-yl)-3-cyclopropyl-1H- pyrazol-1-yl)cyclobutyl)methyl)amino)- 2-(2,6-dioxopiperidin-3-yl)isoindoline- 1,3-dione |
| GS-610 | 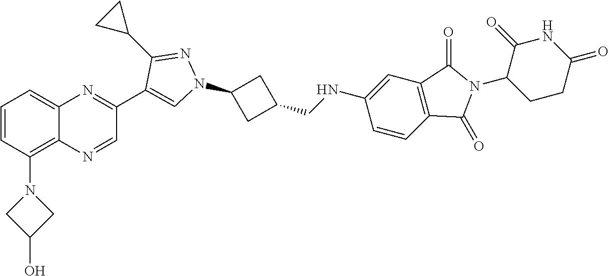 [SMILES: O=C1CCC(N2C(=O)c3ccc(NC[C@H]4C[C@H](n5cc(-c6cnc7c(N8CC(O)C8)cccc7n6)c(C6CC6)n5)C4)cc3C2=O)C(=O)N1] | 5-(((trans-3-(3-cyclopropyl-4-(5-(3- hydroxyazetidin-1-yl)quinoxalin-2-yl)- 1H-pyrazol-1- yl)cyclobutyl)methyl)amino)-2-(2,6- dioxopiperidin-3-yl)isoindoline-1,3-dione |
| GS-611 | 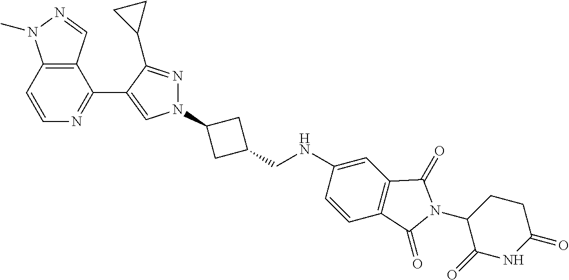 [SMILES: Cn1ncc2c(-c3cn([C@H]4C[C@H](CNc5ccc6c(c5)C(=O)N(C5CCC(=O)NC5=O)C6=O)C4)nc3C3CC3)nccc21] | 5-(((trans-3-(3-cyclopropyl-4-(1-methyl- 1H-pyrazolo[4,3-c]pyridin-4-yl)-1H- pyrazol-1-yl)cyclobutyl)methyl)amino)- 2-(2,6-dioxopiperidin-3-yl)isoindoline- 1,3-dione |
| GS-612 | 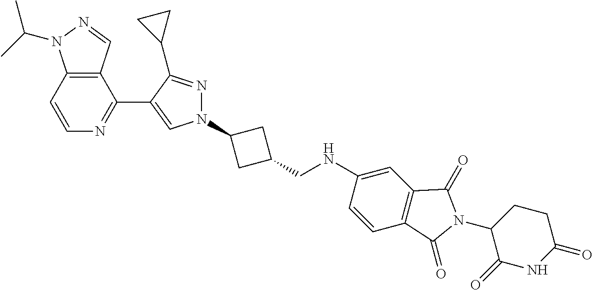 [SMILES: CC(C)n1ncc2c(-c3cn([C@H]4C[C@H](CNc5ccc6c(c5)C(=O)N(C5CCC(=O)NC5=O)C6=O)C4)nc3C3CC3)nccc21] | 5-(((trans-3-(3-cyclopropyl-4-(1- isopropyl-1H-pyrazolo[4,3-c]pyridin-4- yl)-1H-pyrazol-1- yl)cyclobutyl)methyl)amino)-2-(2,6- dioxopiperidin-3-yl)isoindoline-1,3-dione |
| GS-613 | 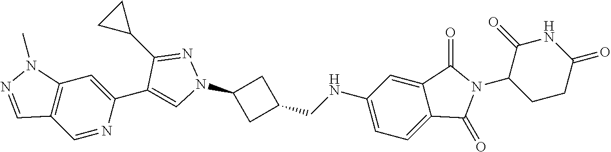 [SMILES: Cn1ncc2cnc(-c3cn([C@H]4C[C@H](CNc5ccc6c(c5)C(=O)N(C5CCC(=O)NC5=O)C6=O)C4)nc3C3CC3)cc21] | 5-(((trans-3-(3-cyclopropyl-4-(1-methyl- 1H-pyrazolo[4,3-c]pyridin-6-yl)-1H- pyrazol-1-yl)cyclobutyl)methyl)amino)- 2-(2,6-dioxopiperidin-3-yl)isoindoline- 1,3-dione |
| GS-614 | 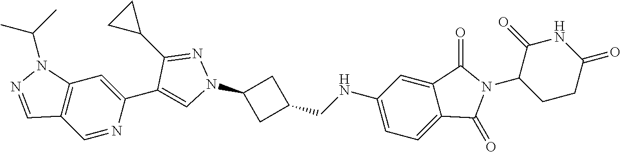 [SMILES: CC(C)n1ncc2cnc(-c3cn([C@H]4C[C@H](CNc5ccc6c(c5)C(=O)N(C5CCC(=O)NC5=O)C6=O)C4)nc3C3CC3)cc21] | 5-(((trans-3-(3-cyclopropyl-4-(1- isopropyl-1H-pyrazolo[4,3-c]pyridin-6- yl)-1H-pyrazol-1- yl)cyclobutyl)methyl)amino)-2-(2,6- dioxopiperidin-3-yl)isoindoline-1,3-dione |
| GS-615 | 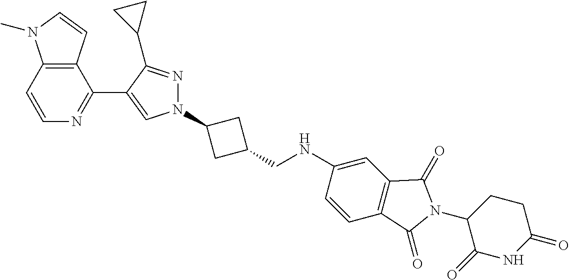 [SMILES: Cn1ccc2c(-c3cn([C@H]4C[C@H](CNc5ccc6c(c5)C(=O)N(C5CCC(=O)NC5=O)C6=O)C4)nc3C3CC3)nccc21] | 5-(((trans-3-(3-cyclopropyl-4-(1-methyl- 1H-pyrrolo[3,2-c]pyridin-4-yl)-1H- pyrazol-1-yl)cyclobutyl)methyl)amino)- 2-(2,6-dioxopiperidin-3-yl)isoindoline- 1,3-dione |
| GS-616 | 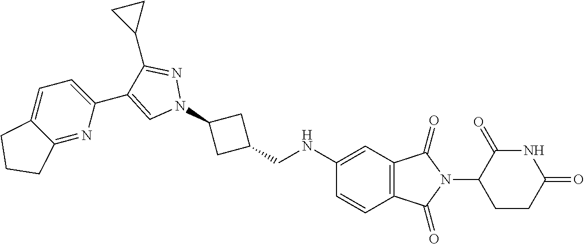 [SMILES: O=C1CCC(N2C(=O)c3ccc(NC[C@H]4C[C@H](n5cc(-c6ccc7c(n6)CCC7)c(C6CC6)n5)C4)cc3C2=O)C(=O)N1] | 5-(((trans-3-(3-cyclopropyl-4-(6,7- dihydro-5H-cyclopenta[b]pyridin-2-yl)- 1H-pyrazol-1- NHclobutyl)methyl)amino)-2-(2,6- dioDeridin-3-yl)isoindoline-1,3-dione |
| GS-617 | 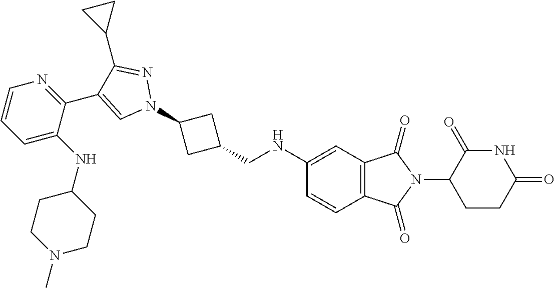 [SMILES: CN1CCC(Nc2cccnc2-c2cn([C@H]3C[C@H](CNc4ccc5c(c4)C(=O)N(C4CCC(=O)NC4=O)C5=O)C3)nc2C2CC2)CC1] | 5-(((trans-3-(3-cyclopropyl-4-(3-((1- methylpiperidin-4-yl)amino)pyridin-2- yl)-1H-pyrazol-1- yl)cyclobutyl)methyl)amino)-2-(2,6- dioxopiperidin-3-yl)isoindoline-1,3-dione |
| GS-618 | 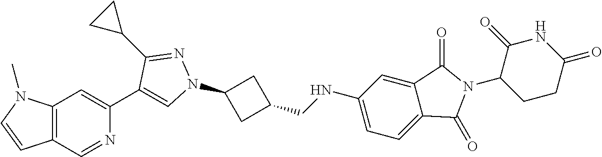 [SMILES: Cn1ccc2cnc(-c3cn([C@H]4C[C@H](CNc5ccc6c(c5)C(=O)N(C5CCC(=O)NC5=O)C6=O)C4)nc3C3CC3)cc21] | 5-(((trans-3-(3-cyclopropyl-4-(1-methyl- 1H-pyrrolo[3,2-c]pyridin-6-yl)-1H- pyrazol-1-yl)cyclobutyl)methyl)amino)- 2-(2,6-dioxopiperidin-3-yl)isoindoline- 1,3-dione |
| GS-619 | 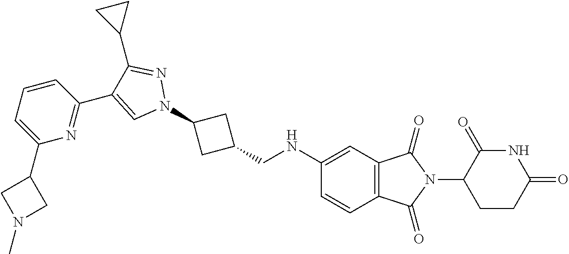 [SMILES: CN1CC(c2cccc(-c3cn([C@H]4C[C@H](CNc5ccc6c(c5)C(=O)N(C5CCC(=O)NC5=O)C6=O)C4)nc3C3CC3)n2)C1] | 5-(((trans-3-(3-cyclopropyl-4-(6-(1- methylazetidin-3-yl)pyridin-2-yl)-1H- pyrazol-1-yl)cyclobutyl)methyl)amino)- 2-(2,6-dioxopiperidin-3-yl)isoindoline- 1,3-dione |
| GS-620 | 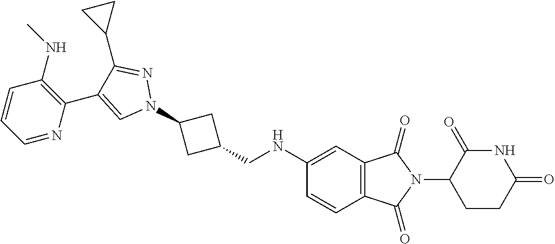 [SMILES: CNc1cccnc1-c1cn([C@H]2C[C@H](CNc3ccc4c(c3)C(=O)N(C3CCC(=O)NC3=O)C4=O)C2)nc1C1CC1] | 5-(((trans-3-(3-cyclopropyl-4-(3- (methylamino)pyridin-2-yl)-1H-pyrazol- 1-yl)cyclobutyl)methyl)amino)-2-(2,6- dioxopiperidin-3-yl)isoindoline-1,3-dione |
| GS-621 | 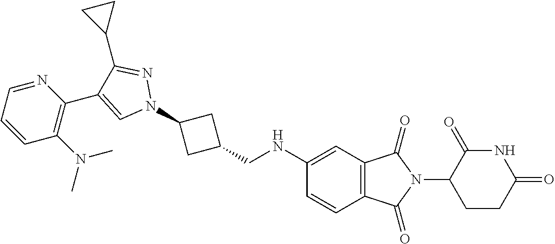 [SMILES: CN(C)c1cccnc1-c1cn([C@H]2C[C@H](CNc3ccc4c(c3)C(=O)N(C3CCC(=O)NC3=O)C4=O)C2)nc1C1CC1] | 5-(((trans-3-(3-cyclopropyl-4-(3- (dimethylamino)pyridin-2-yl)-1H- pyrazol-1-yl)cyclobutyl)methyl)amino)- 2-(2,6-dioxopiperidin-3-yl)isoindoline- 1,3-dione |
| GS-622 | 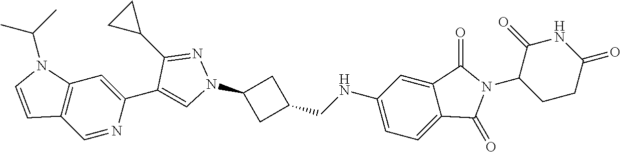 [SMILES: CC(C)n1ccc2cnc(-c3cn([C@H]4C[C@H](CNc5ccc6c(c5)C(=O)N(C5CCC(=O)NC5=O)C6=O)C4)nc3C3CC3)cc21] | 5-(((trans-3-(3-cyclopropyl-4-(1- isopropyl-1H-pyrrolo[3,2-c]pyridin-6-yl)- 1H-pyrazol-1- yl)cyclobutyl)methyl)amino)-2-(2,6- dioxopiperidin-3-yl)isoindoline-1,3-dione |
| GS-623 | 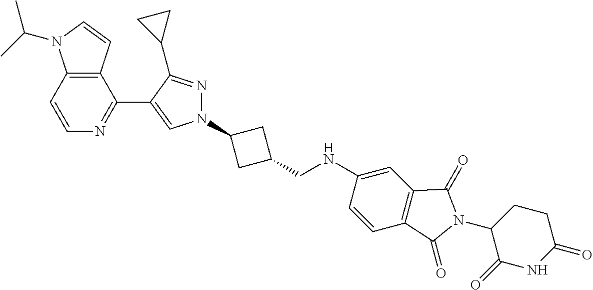 [SMILES: CC(C)n1ccc2c(-c3cn([C@H]4C[C@H](CNc5ccc6c(c5)C(=O)N(C5CCC(=O)NC5=O)C6=O)C4)nc3C3CC3)nccc21] | 5-(((trans-3-(3-cyclopropyl-4-(1- isopropyl-1H-pyrrolo[3,2-c]pyridin-4-yl)- 1H-pyrazol-1- yl)cyclobutyl)methyl)amino)-2-(2,6- dioxopiperidin-3-yl)isoindoline-1,3-dione |
| GS-624 | 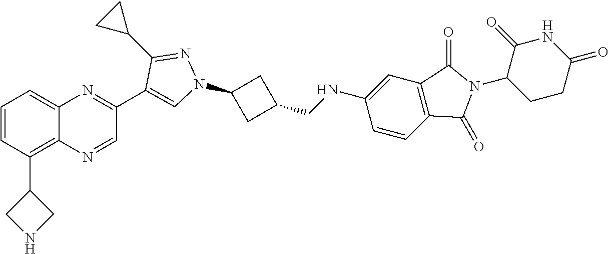 [SMILES: O=C1CCC(N2C(=O)c3ccc(NC[C@H]4C[C@H](n5cc(-c6cnc7c(C8CNC8)cccc7n6)c(C6CC6)n5)C4)cc3C2=O)C(=O)N1] | 5-(((trans-3-(4-(5-(azetidin-3- yl)quinoxalin-2-yl)-3-cyclopropyl-1H- pyrazol-1-yl)cyclobutyl)methyl)amino)- 2-(2,6-dioxopiperidin-3-yl)isoindoline- 1,3-dione |
| GS-625 | 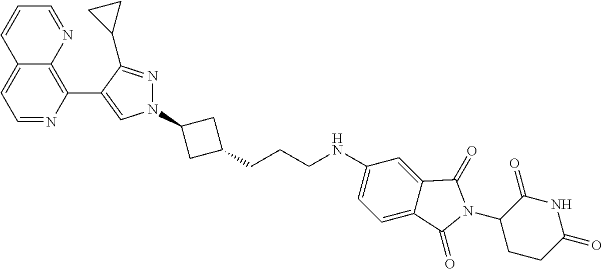 [SMILES: O=C1CCC(N2C(=O)c3ccc(NCCC[C@H]4C[C@H](n5cc(-c6nccc7cccnc67)c(C6CC6)n5)C4)cc3C2=O)C(=O)N1] | 5-((3-(trans-3-(3-cyclopropyl-4-(1,7- naphthyridin-8-yl)-1H-pyrazol-1- yl)cyclobutyl)propyl)amino)-2-(2,6- dioxopiperidin-3-yl)isoindoline-1,3-dione |
| GS-626 | 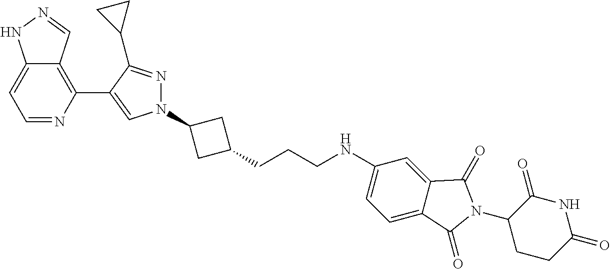 [SMILES: O=C1CCC(N2C(=O)c3ccc(NCCC[C@H]4C[C@H](n5cc(-c6nccc7[nH]ncc67)c(C6CC6)n5)C4)cc3C2=O)C(=O)N1] | 5-((3-(trans-3-(3-cyclopropyl-4-(1H- pyrazolo[4,3-c]pyridin-4-yl)-1H-pyrazol- 1-yl)cyclobutyl)propyl)amino)-2-(2,6- dioxopiperidin-3-yl)isoindoline-1,3-dione |
| GS-627 | 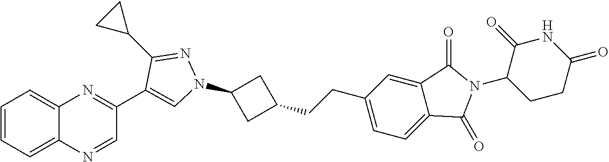 [SMILES: O=C1CCC(N2C(=O)c3ccc(CC[C@H]4C[C@H](n5cc(-c6cnc7ccccc7n6)c(C6CC6)n5)C4)cc3C2=O)C(=O)N1] | 5-(2-(trans-3-(3-cyclopropyl-4- (quinoxalin-2-yl)-1H-pyrazol-1- yl)cyclobutyl)ethyl)-2-(2,6- dioxopiperidin-3-yl)isoindoline-1,3-dione |
| GS-628 | 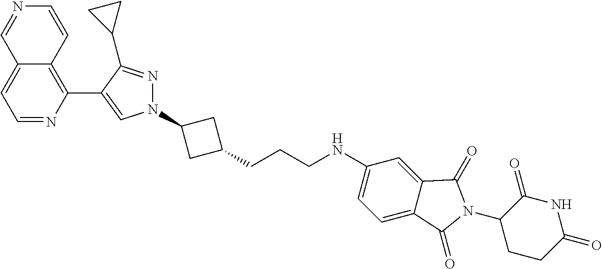 [SMILES: O=C1CCC(N2C(=O)c3ccc(NCCC[C@H]4C[C@H](n5cc(-c6nccc7cnccc67)c(C6CC6)n5)C4)cc3C2=O)C(=O)N1] | 5-((3-(trans-3-(3-cyclopropyl-4-(2,6- naphthyridin-1-yl)-1H-pyrazol-1- yl)cyclobutyl)propyl)amino)-2-(2,6- dioxopiperidin-3-yl)isoindoline-1,3-dione |
| GS-629 | 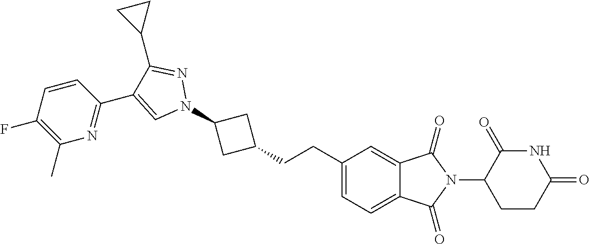 [SMILES: Cc1nc(-c2cn([C@H]3C[C@H](CCc4ccc5c(c4)C(=O)N(C4CCC(=O)NC4=O)C5=O)C3)nc2C2CC2)ccc1F] | 5-(2-(trans-3-(3-cyclopropyl-4-(5-fluoro- 6-methylpyridin-2-yl)-1H-pyrazol-1- yl)cyclobutyl)ethyl)-2-(2,6- dioxopiperidin-3-yl)isoindoline-1,3-dione |
| GS-630 | 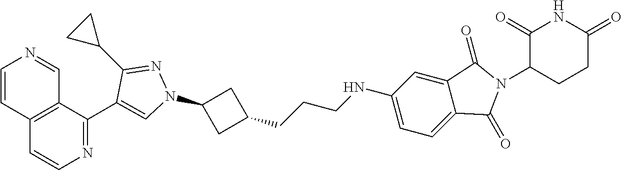 [SMILES: O=C1CCC(N2C(=O)c3ccc(NCCC[C@H]4C[C@H](n5cc(-c6nccc7ccncc67)c(C6CC6)n5)C4)cc3C2=O)C(=O)N1] | 5-((3-(trans-3-(3-cyclopropyl-4-(2,7- naphthyridin-1-yl)-1H-pyrazol-1- yl)cyclobutyl)propyl)amino)-2-(2,6- dioxopiperidin-3-yl)isoindoline-1,3-dione |
| GS-631 | 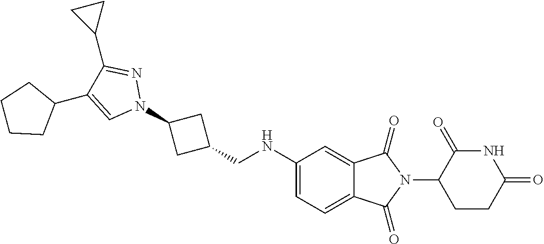 [SMILES: O=C1CCC(N2C(=O)c3ccc(NC[C@H]4C[C@H](n5cc(C6CCCC6)c(C6CC6)n5)C4)cc3C2=O)C(=O)N1] | 5-(((trans-3-(4-cyclopentyl-3- cyclopropyl-1H-pyrazol-1- yl)cyclobutyl)methyl)amino)-2-(2,6- dioxopiperidin-3-yl)isoindoline-1,3-dione |
| GS-632 | 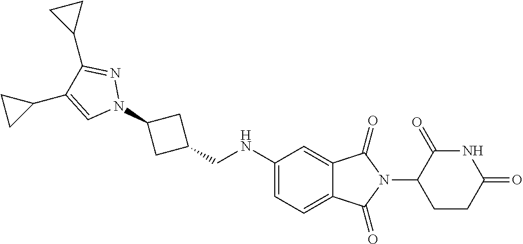 [SMILES: O=C1CCC(N2C(=O)c3ccc(NC[C@H]4C[C@H](n5cc(C6CC6)c(C6CC6)n5)C4)cc3C2=O)C(=O)N1] | 5-(((trans-3-(3,4-dicyclopropyl-1H- pyrazol-1-yl)cyclobutyl)methyl)amino)- 2-(2,6-dioxopiperidin-3-yl)isoindoline- 1,3-dione |
| GS-633 | 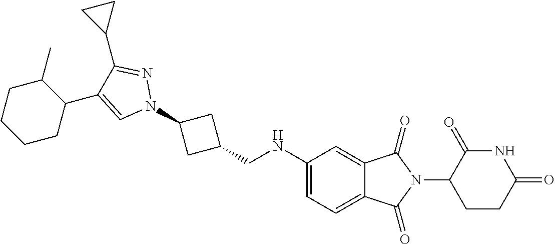 [SMILES: CC1CCCCC1c1cn([C@H]2C[C@H](CNc3ccc4c(c3)C(=O)N(C3CCC(=O)NC3=O)C4=O)C2)nc1C1CC1] | 5-(((trans-3-(3-cyclopropyl-4-(2- methylcyclohexyl)-1H-pyrazol-1- yl)cyclobutyl)methyl)amino)-2-(2,6- dioxopiperidin-3-yl)isoindoline-1,3-dione |
| GS-634 | 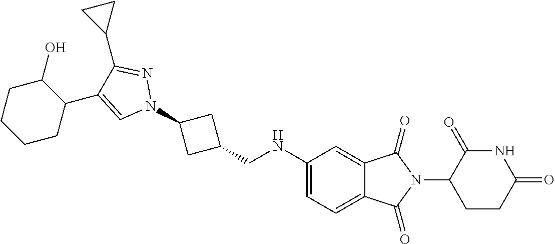 [SMILES: O=C1CCC(N2C(=O)c3ccc(NC[C@H]4C[C@H](n5cc(C6CCCCC6O)c(C6CC6)n5)C4)cc3C2=O)C(=O)N1] | 5-(((trans-3-(3-cyclopropyl-4-(2- hydroxycyclohexyl)-1H-pyrazol-1- yl)cyclobutyl)methyl)amino)-2-(2,6- dioxopiperidin-3-yl)isoindoline-1,3-dione |
| GS-635 | 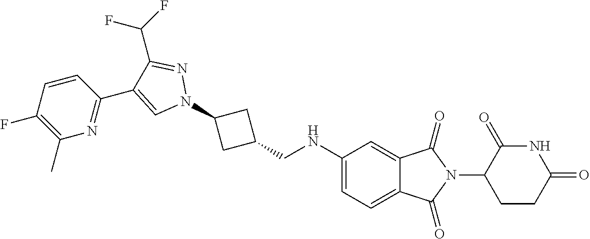 [SMILES: Cc1nc(-c2cn([C@H]3C[C@H](CNc4ccc5c(c4)C(=O)N(C4CCC(=O)NC4=O)C5=O)C3)nc2C(F)F)ccc1F] | 5-(((trans-3-(3-(difluoromethyl)-4-(5- fluoro-6-methylpyridin-2-yl)-1H-pyrazol- 1-yl)cyclobutyl)methyl)amino)-2-(2,6- dioxopiperidin-3-yl)isoindoline-1,3-dione |
| GS-636 | 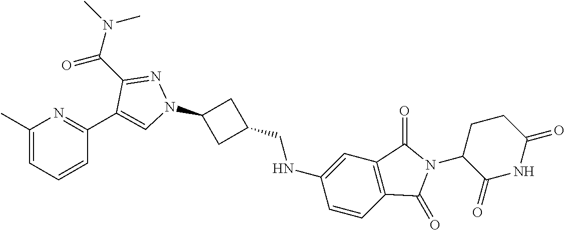 [SMILES: Cc1cccc(-c2cn([C@H]3C[C@H](CNc4ccc5c(c4)C(=O)N(C4CCC(=O)NC4=O)C5=O)C3)nc2C(=O)N(C)C)n1] | 1-(trans-3-(((2-(2,6-dioxopiperidin-3-yl)- 1,3-dioxoisoindolin-5- yl)amino)methyl)cyclobutyl)-N,N- dimethyl-4-(6-methylpyridin-2-yl)-1H- pyrazole-3-carboxamide |
| GS-637 | 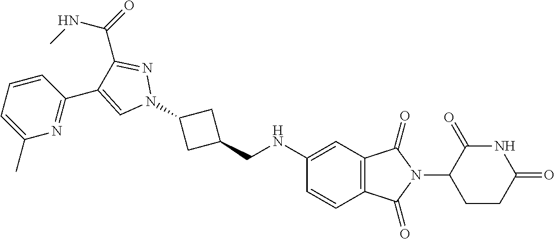 [SMILES: CNC(=O)c1nn([C@H]2C[C@H](CNc3ccc4c(c3)C(=O)N(C3CCC(=O)NC3=O)C4=O)C2)cc1-c1cccc(C)n1] | 1-(trans-3-(((2-(2,6-dioxopiperidin-3-yl)- 1,3-dioxoisoindolin-5- yl)amino)methyl)cyclobutyl)-N-methyl- 4-(6-methylpyridin-2-yl)-1H-pyrazole-3- carboxamide |
| GS-638 | 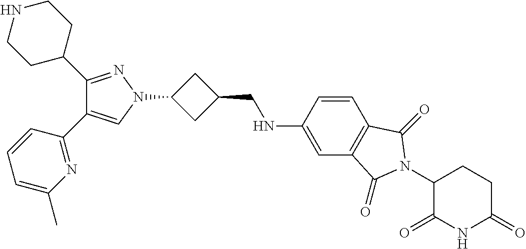 [SMILES: Cc1cccc(-c2cn([C@H]3C[C@H](CNc4ccc5c(c4)C(=O)N(C4CCC(=O)NC4=O)C5=O)C3)nc2C2CCNCC2)n1] | 2-(2,6-dioxopiperidin-3-yl)-5-(((trans-3- (4-(6-methylpyridin-2-yl)-3-(piperidin-4- yl)-1H-pyrazol-1- yl)cyclobutyl)methyl)amino)isoindoline- 1,3-dione |
| GS-639 | 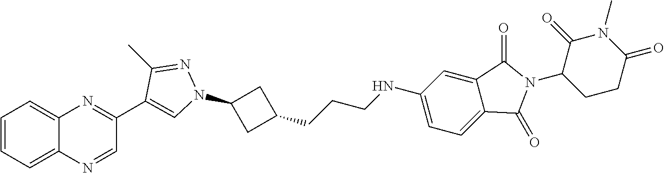 [SMILES: Cc1nn([C@H]2C[C@H](CCCNc3ccc4c(c3)C(=O)N(C3CCC(=O)N(C)C3=O)C4=O)C2)cc1-c1cnc2ccccc2n1] | 2-(1-methyl-2,6-dioxopiperidin-3-yl)-5- ((3-(trans-3-(3-methyl-4-(quinoxalin-2- yl)-1H-pyrazol-1- yl)cyclobutyl)propyl)amino)isoindoline- 1,3-dione |

As used herein, in case of discrepancy between the structure an chemical name provided for a particular compound, the structure shall control.

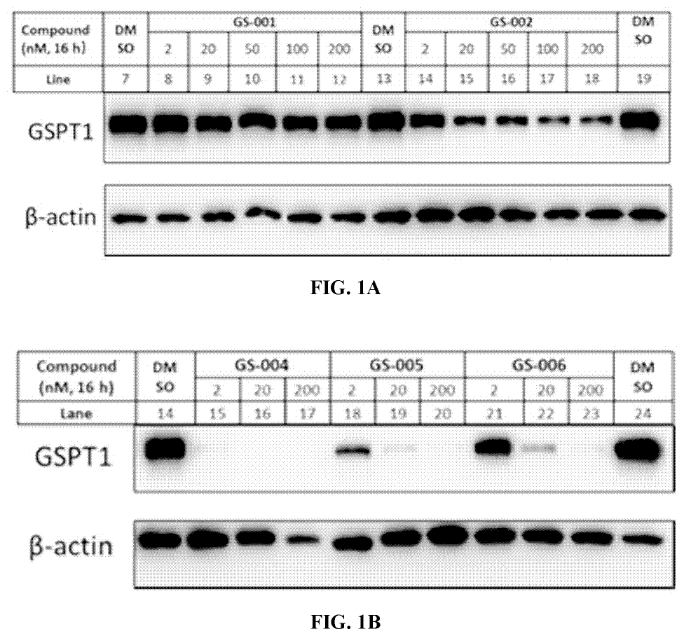

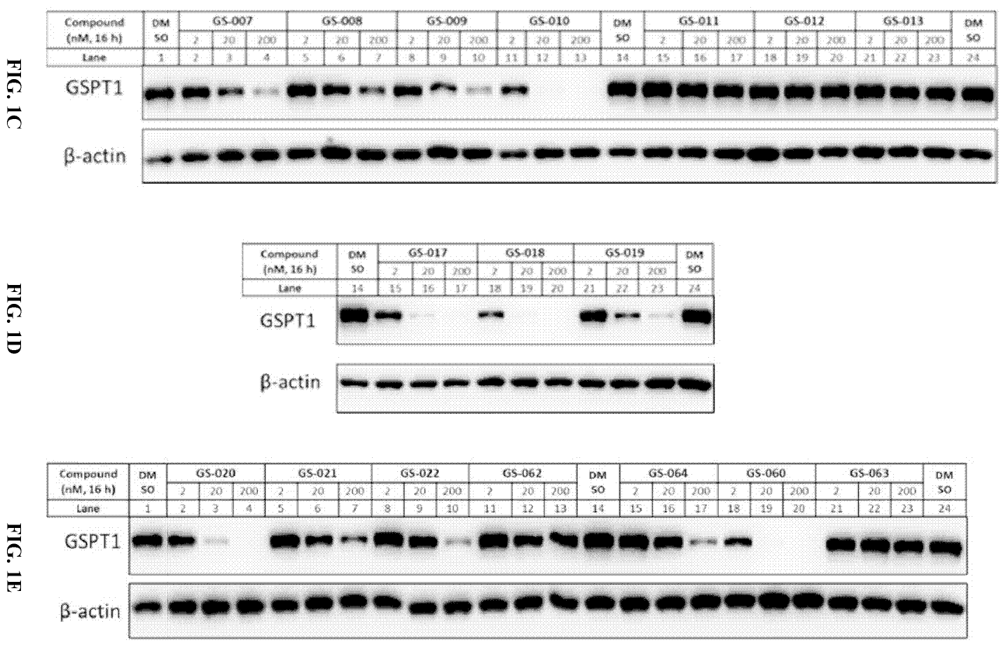

### Example 626. Selected GSPT1 Degraders Concentration-Dependently Reduced GSPT1 Protein Levels in MV4;11 Cells (FIG. 1A-1E)

MV4;11 cells were treated with compounds at concentrations as shown in FIG. 1A-IE for 16 h. Data showed that GSPT1 proteins levels were reduced by some compounds in a concentration-dependent manner, thus indicating that they were GSPT1 degraders.

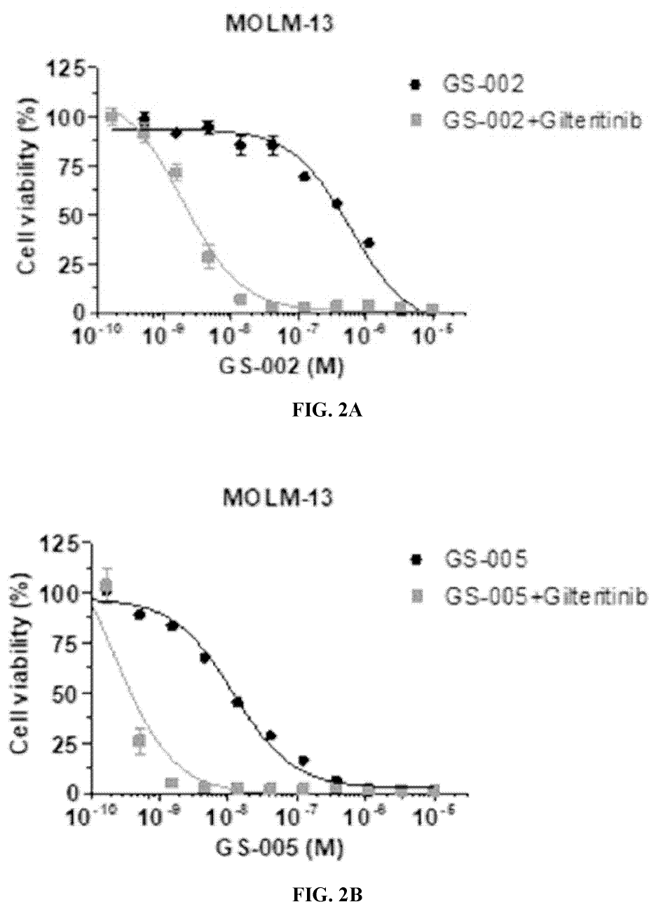

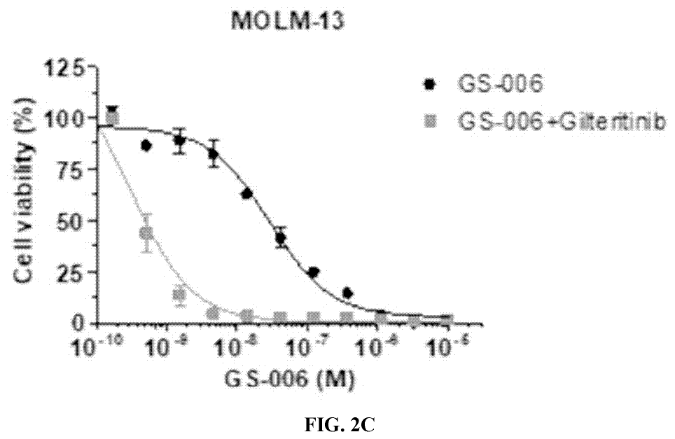

### Example 627. Inhibition of FLT3 in FLT3-Mutant AML Cells Remarkably Increases Sensitivity to GSPT1 Degraders (FIG. 2A-2C)

MOLM-13 cells were treated with GSPT1 degraders at indicated concentrations with or without 20 nM gilteritinib for 3 days. Cell viability and dose response curves were determined as described in the Materials and Methods subsection. FIG. 2A-2C show that inhibition of FLT3 in FLT3-mutant AML cells remarkably increases sensitivity to GSPT1 degraders, GS-002, GS-005, and GS-006.

### Materials and Methods:

### General Chemistry Methods:

All chemicals and reagents were purchased from commercial suppliers and used without further purification. LCMS spectra for all compounds were acquired using a Waters LC-MS AcQuity H UPLC class system. The Waters LC-MS AcQuity H UPLC class system comprising a pump (Quatemnary Solvent Manager) with degasser, an autosampler (FTN), a column oven (40° C., unless otherwise indicated), a photo-diode array PDA detector. Chromatography was performed on an AcQuity UPLC BEH C18 (1.7 μm, 2.1×50 mm) with water containing 0.1% formic acid as solvent A and acetonitrile containing 0.1% formic acid as solvent B at a flow rate of 0.6 mL/min. Flow from the column was split to a MS spectrometer. The MS detector was configured with an electrospray ionization source. Nitrogen was used as the nebulizer gas. Data acquisition was performed with a MassLynx data system. Nuclear Magnetic Resonance spectra were recorded on a Bruker Avance III400 spectrometer. Chemical shifts are expressed in parts per million (ppm) and reported as δ value (chemical shift δ). Coupling constants are reported in units of hertz (J value, Hz; Integration and splitting patterns: where s=singlet, d=double, t=triplet, q=quartet, brs=broad singlet, m=multiple). The purification of intermediates or final products were performed on Agilent Prep 1260 series with UV detector set to 254 nm or 220 nm. Samples were injected onto a Phenomenex Luna C18 column (5 μm, 30×75 mm,) at rt. The flow rate was 40 mL/min. A linear gradient was used with either 10% or 50% MeOH in H₂O containing 0.1% TFA as solvent A and 100% of MeOH as solvent B. Alternatively, the products were purified on CombiFlash® NextGen 300 system with UV detector set to 254 nm, 220 nm or 280 nm. The flow rate was 40 mL/min. A linear gradient was used with H₂O containing 0.05% TFA as solvent A and 100% of MeOH containing 0.05% TFA as solvent B. All compounds showed >95% purity using the LCMS methods described above.

### Cell Culture

RS4;11, MV4;11, MOLM-13 and other cells were cultured at 37° C. with 5% CO₂ in RPMI 1640 or DMEM Medium supplemented with 10% fetal bovine serum. Cells were authenticated using the short tandem repeat (STR) assays. Mycoplasma test results were negative.

### Antibodies and Reagents

Rabbit anti-FLT3 antibody (3462S) was purchased from Cell Signaling Technology. Rabbit anti-GSPT1 antibody (ab126090) was purchased from Abcam. HRP-conjugated anti-α-tubulin antibody was produced in house. Media and other cell culture reagents were purchased from Thermo Fisher. The CellTiter-Glo Luminescent Assay kit was purchased from Promega.

### Immunoblotting

Cultured cells were washed with cold PBS once and lysed in cold RIPA buffer supplemented with protease inhibitors and phosphatase inhibitors (Beyotime Biotechnology). The solutions were then incubated at 4° C. for 30 minutes with gentle agitation to fully lyse cells. Cell lysates were centrifuged at 13,000 rpm for 10 minutes at 4° C. and pellets were discarded. Total protein concentrations in the lysates were determined by BCA assays (Beyotime Biotechnology). Cell lysates were mixed with Laemmli loading buffer to 1× and heated at 99° C. for 5 min. Proteins were resolved on SDS-PAGE and visualized by chemiluminescence. Images were taken by a ChemiDoc MP Imaging system (Bio-Rad). Protein bands were quantitated using the accompanied software provided by Bio-Rad.

### Cell Viability Assays

Cells were seeded at a density of 5000 cells per well in 96-well assay plates and treated with test compounds following a 8-point or 12-point 3-fold serial dilution. Three days later, cell viability was determined using the CellTiter-Glo assay kit according to the manufacturer's instructions. The dose-response curves were determined and IC₅₀ values were calculated using the GraphiPad Prism software following a nonlinear regression (least squares fit) method.

The cell viability inhibition results of selected compounds are set forth in Tables 2 and 3 below. The compounds were tested in both MV4;11 cells and in MOLM-13 cells, either alone or in combination with gilteritinib.

TABLE 2

| | |
|---|---|
| MV4; 11 IC₅₀ | MOLM-13 IC₅₀ |

| ID | | gilteritinib | | gilteritinib |
|---|---|---|---|---|
| CC-90009 | 54 | 7.1 | 103 | 8.6 |
| gilteritinib | 2.3 | | 7 | |
| GS-001 | 4743 | >10000 | 301 | 27 |
| GS-002 | 227 | 3 | 583 | 1.9 |
| GS-003 | >10000 | 191 | >10000 | 103 |
| GS-004 | 1.3 | 0.5 | 2.1 | 0.2 |
| GS-005 | 6 | 1.8 | 11.8 | 0.2 |
| GS-006 | 12 | 4.6 | 29 | 0.3 |
| GS-007 | 170 | 9.9 | 179 | 32 |
| GS-008 | 449 | 27 | 78 | 14 |
| GS-009 | 296 | 22 | 35 | 18 |
| GS-010 | 17 | 2.3 | 3 | 0.3 |
| GS-011 | >10000 | >10000 | >10000 | 270 |
| GS-012 | >10000 | >10000 | >10000 | 312 |
| GS-013 | >10000 | >10000 | >10000 | 1848 |
| GS-014 | 71 | 8.3 | 107 | 10 |
| GS-015 | >10000 | 2783 | >10000 | 30 |
| GS-016 | >10000 | >10000 | >10000 | 86 |
| GS-017 | 20 | 2 | 30 | 2 |
| GS-018 | 8.7 | 0.9 | 15.6 | 1.6 |
| GS-019 | 162 | 9.4 | 146 | 7 |
| GS-020 | 29 | 3 | 87 | 11 |
| GS-021 | 3133 | 44 | 82 | 111 |
| GS-022 | 293 | 60 | 875 | 106 |
| GS-023 | | | | |
| GS-024 | | | | |
| GS-025 | | | | |
| GS-026 | | | | |
| GS-027 | | | | |
| GS-028 | | | | |
| GS-029 | | | | |
| GS-030 | | | | |
| GS-031 | | | | |
| GS-032 | | | | |
| GS-033 | | | | |
| GS-034 | | | | |
| GS-035 | | | | |
| GS-036 | | | | |
| GS-037 | | | | |
| GS-038 | | | | |
| GS-039 | | | | |
| GS-040 | | | | |
| GS-041 | | | | |
| GS-042 | | | | |
| GS-043 | | | | |
| GS-044 | | | | |
| GS-045 | | | | |
| GS-046 | | | | |
| GS-047 | | | | |
| GS-048 | | | | |
| GS-049 | | | | |
| GS-050 | | | | |
| GS-051 | | | | |
| GS-052 | | | | |
| GS-053 | | | | |
| GS-054 | | | | |
| GS-055 | | | | |
| GS-056 | | | | |
| GS-057 | | | | |
| GS-058 | | | | |
| GS-059 | | | | |
| GS-060 | <1 | | 3.7 | 0.06 |
| GS-061 | | | | |
| GS-062 | 9409 | 1917 | >10000 | 72 |
| GS-063 | >10000 | 8547 | >10000 | 3680 |
| GS-064 | 349 | 27 | 520 | 600 |

The IC₅₀ values (nM) of each compound were determined in MV4;11 and MOLM-13 cells with or without gilteritinib (10 nM for MV4;11; and 20 nM for MOML-13) as described in the Materials and Methods subsection, and calculated using the GraphPad Prism 5.0 software.

TABLE 3

| | |
|---|---|
| MV4; 11 IC₅₀ | MOLM-13 IC₅₀ |

| ID | | gilteritinib | | gilteritinib |
|---|---|---|---|---|
| GS-025 | B | A | A | A |
| GS-026 | D | D | D | D |
| GS-027 | D | D | C | D |
| GS-028 | B | B | C | B |
| GS-029 | D | D | D | D |
| GS-030 | D | D | D | D |
| GS-031 | B | A | B | B |
| GS-034 | C | A | D | A |
| GS-035 | B | A | D | A |
| GS-036 | D | A | D | B |
| GS-043 | B | A | B | A |
| GS-044 | B | A | B | A |
| GS-045 | C | A | D | A |
| GS-046 | D | B | C | B |
| GS-047 | C | B | C | B |
| GS-048 | B | A | C | B |
| GS-049 | B | B | B | A |
| GS-050 | A | A | B | A |
| GS-051 | B | A | C | B |
| GS-052 | C | A | D | A |
| GS-053 | B | A | C | A |
| GS-054 | A | A | B | A |
| GS-058 | D | C | C | D |
| GS-060 | A | A | A | A |
| GS-061 | D | C | D | C |
| GS-065 | D | D | D | D |
| GS-066 | B | A | B | A |
| GS-067 | B | B | D | B |
| GS-068 | B | A | D | B |
| GS-069 | B | A | C | B |
| GS-070 | B | A | C | A |
| GS-071 | D | B | D | B |
| GS-072 | D | C | D | C |
| GS-073 | C | B | C | C |
| GS-074 | D | D | D | D |
| GS-075 | C | B | C | B |
| GS-076 | B | A | B | A |
| GS-077 | A | A | B | A |
| GS-078 | B | A | B | A |
| GS-079 | C | B | D | C |
| GS-080 | D | C | D | C |
| GS-081 | D | D | D | D |
| GS-082 | D | C | D | C |
| GS-083 | B | B | C | B |
| GS-084 | D | D | D | D |
| GS-085 | D | D | D | D |
| GS-086 | D | C | D | D |
| GS-087 | A | A | A | A |
| GS-088 | D | D | D | D |
| GS-089 | D | D | D | D |
| GS-090 | B | A | B | A |
| GS-091 | D | D | D | D |
| GS-092 | D | D | D | D |
| GS-093 | A | A | A | A |
| GS-094 | D | D | D | D |
| GS-095 | C | B | C | B |
| GS-096 | B | A | C | A |
| GS-097 | B | A | B | A |
| GS-098 | B | B | C | A |
| GS-099 | C | B | C | B |
| GS-100 | D | D | D | C |
| GS-101 | A | A | B | A |
| GS-102 | B | A | C | B |
| GS-103 | D | C | D | C |
| GS-104 | C | C | D | C |
| GS-105 | B | B | B | A |
| GS-106 | C | B | D | B |
| GS-107 | C | B | D | B |
| GS-108 | D | C | D | B |
| GS-109 | C | C | D | C |
| GS-110 | A | A | B | A |
| GS-111 | C | B | D | C |
| GS-112 | D | C | D | C |
| GS-113 | B | A | D | B |
| GS-114 | C | B | C | B |
| GS-115 | C | B | B | A |
| GS-116 | B | A | C | A |
| GS-117 | D | C | D | C |
| GS-118 | D | D | B | A |
| GS-119 | D | B | D | C |
| GS-120 | B | A | B | A |
| GS-121 | C | B | D | C |
| GS-122 | A | A | A | A |
| GS-123 | C | C | D | A |
| GS-124 | D | D | D | B |
| GS-125 | C | C | D | D |
| GS-126 | C | D | D | D |
| GS-127 | D | C | D | C |
| GS-128 | D | D | D | D |
| GS-129 | D | C | D | C |
| GS-130 | D | D | D | D |
| GS-131 | D | D | D | D |
| GS-132 | A | A | A | A |
| GS-133 | D | D | D | D |
| GS-134 | B | B | B | A |
| GS-135 | D | C | D | D |
| GS-136 | D | D | D | D |
| GS-137 | D | D | D | D |
| GS-138 | D | D | D | D |
| GS-139 | C | C | D | D |
| GS-140 | B | C | D | D |
| GS-141 | D | D | D | D |
| GS-142 | D | D | D | D |
| GS-143 | D | D | D | D |
| GS-144 | C | B | D | C |
| GS-145 | C | B | C | A |
| GS-146 | D | B | C | B |
| GS-147 | D | C | D | B |
| GS-148 | D | C | C | B |
| GS-149 | D | C | D | C |
| GS-150 | C | B | C | A |
| GS-151 | B | A | B | A |
| GS-152 | C | B | C | B |
| GS-153 | C | C | D | B |
| GS-154 | D | D | D | D |
| GS-155 | C | D | D | C |
| GS-156 | D | C | D | A |
| GS-157 | D | D | D | D |
| GS-158 | D | D | D | D |
| GS-159 | D | D | D | D |
| GS-160 | C | B | D | B |
| GS-161 | C | B | D | B |
| GS-162 | D | D | D | B |
| GS-163 | C | B | C | A |
| GS-164 | C | B | C | B |
| GS-165 | C | B | C | B |
| GS-166 | B | A | B | A |
| GS-167 | D | C | D | A |
| GS-168 | C | B | D | B |
| GS-169 | C | D | D | B |
| GS-170 | D | B | D | B |
| GS-171 | B | A | B | A |
| GS-172 | C | D | D | C |
| GS-173 | B | B | B | A |
| GS-174 | C | B | D | A |
| GS-175 | C | B | C | A |
| GS-176 | C | B | C | B |
| GS-177 | D | C | D | B |
| GS-178 | B | B | C | B |
| GS-179 | D | D | D | C |
| GS-180 | C | B | C | B |
| GS-181 | B | A | B | A |
| GS-182 | C | B | C | A |
| GS-183 | C | C | D | C |
| GS-184 | C | D | D | C |
| GS-185 | C | B | C | B |
| GS-186 | D | D | D | C |
| GS-187 | D | D | D | D |
| GS-188 | B | A | B | A |
| GS-189 | C | D | C | C |
| GS-190 | D | D | D | C |
| GS-191 | B | A | B | A |
| GS-192 | C | B | D | B |
| GS-193 | D | C | C | C |
| GS-194 | B | A | C | A |
| GS-195 | C | A | C | A |
| GS-196 | B | A | B | A |
| GS-197 | B | B | C | B |
| GS-198 | B | B | C | A |
| GS-199 | B | A | C | A |
| GS-200 | C | B | C | B |
| GS-201 | C | B | C | B |
| GS-202 | B | A | C | A |
| GS-203 | C | B | D | B |
| GS-204 | B | B | C | A |
| GS-205 | B | A | B | A |
| GS-206 | B | B | C | B |
| GS-207 | C | B | C | A |
| GS-208 | C | B | C | B |
| GS-209 | C | B | C | B |
| GS-210 | A | A | A | A |
| GS-211 | B | A | B | A |
| GS-212 | B | B | B | B |
| GS-213 | A | A | A | A |
| GS-214 | C | B | D | B |
| GS-215 | C | B | C | A |
| GS-216 | C | D | D | D |
| GS-217 | B | A | C | A |
| GS-218 | C | B | C | B |
| GS-219 | C | B | D | B |
| GS-220 | D | C | D | C |
| GS-221 | A | A | A | A |
| GS-222 | D | C | D | B |
| GS-223 | A | A | A | A |
| GS-224 | C | B | C | A |
| GS-225 | C | B | D | B |
| GS-226 | C | C | D | D |
| GS-227 | A | A | A | A |
| GS-228 | A | A | A | A |
| GS-229 | B | A | B | A |
| GS-230 | B | A | B | A |
| GS-231 | B | A | B | A |
| GS-232 | C | C | C | D |
| GS-233 | C | B | C | B |
| GS-234 | A | B | C | B |
| GS-235 | A | C | D | C |
| GS-236 | C | B | C | B |
| GS-237 | D | C | D | C |
| GS-238 | C | B | B | B |
| GS-239 | C | B | D | B |
| GS-240 | B | A | C | B |
| GS-241 | C | C | D | C |
| GS-242 | D | C | D | C |
| GS-243 | B | A | B | A |
| GS-244 | C | B | D | B |
| GS-245 | D | B | D | C |
| GS-246 | C | B | D | C |
| GS-247 | C | A | D | B |
| GS-248 | B | B | D | B |
| GS-249 | C | D | D | C |
| GS-250 | C | B | C | B |
| GS-251 | C | B | C | B |
| GS-252 | C | D | D | D |
| GS-253 | A | A | B | A |
| GS-254 | B | A | B | A |
| GS-255 | B | A | C | A |
| GS-256 | B | A | C | A |
| GS-257 | A | A | B | A |
| GS-258 | C | B | D | B |
| GS-259 | A | A | B | A |
| GS-260 | D | D | C | D |
| GS-261 | A | A | A | A |
| GS-262 | A | A | A | A |
| GS-263 | C | B | C | B |
| GS-264 | B | A | B | A |
| GS-265 | D | C | D | C |
| GS-266 | A | A | A | A |
| GS-267 | A | A | A | A |
| GS-268 | C | B | C | B |
| GS-269 | D | C | D | D |
| GS-270 | D | D | D | D |
| GS-271 | B | B | C | B |
| GS-272 | A | A | B | A |
| GS-273 | C | B | D | C |
| GS-274 | D | D | D | D |
| GS-275 | B | B | C | B |
| GS-276 | B | A | C | B |
| GS-277 | A | A | A | A |
| GS-278 | A | A | B | A |
| GS-279 | C | B | D | B |
| GS-280 | A | A | A | A |
| GS-281 | A | A | B | A |
| GS-282 | B | A | C | B |
| GS-283 | C | B | D | B |
| GS-284 | A | A | A | A |
| GS-285 | B | A | C | B |
| GS-286 | A | A | A | A |
| GS-287 | A | A | A | A |
| GS-288 | C | B | C | B |
| GS-289 | B | A | C | B |
| GS-290 | D | B | D | C |
| GS-291 | D | B | D | B |
| GS-292 | D | D | D | D |
| GS-293 | C | B | D | C |
| GS-294 | D | D | D | D |
| GS-295 | D | D | D | D |
| GS-296 | B | B | C | B |
| GS-297 | D | D | D | D |
| GS-298 | D | D | D | D |
| GS-299 | A | A | B | A |
| GS-300 | D | D | D | D |
| GS-301 | D | C | D | C |
| GS-302 | D | D | D | D |
| GS-303 | D | D | D | D |
| GS-304 | D | D | D | D |
| GS-305 | B | A | B | A |
| GS-306 | D | D | D | D |
| GS-307 | C | B | C | B |
| GS-308 | B | A | B | A |
| GS-309 | A | A | A | A |
| GS-310 | B | B | C | B |
| GS-311 | B | A | D | B |
| GS-312 | D | D | D | D |
| GS-313 | D | D | D | D |
| GS-314 | B | A | B | A |
| GS-315 | B | A | C | B |
| GS-316 | D | D | D | C |
| GS-317 | D | D | D | C |
| GS-318 | C | B | D | B |
| GS-319 | D | D | D | B |
| GS-320 | D | D | D | C |
| GS-321 | D | D | D | C |
| GS-322 | D | D | D | D |
| GS-323 | C | A | D | A |
| GS-324 | D | C | D | B |
| GS-325 | D | C | D | C |
| GS-326 | D | D | D | C |
| GS-327 | D | C | D | D |
| GS-328 | C | B | D | B |
| GS-329 | D | D | D | C |
| GS-330 | D | D | D | D |
| GS-331 | D | D | D | C |
| GS-332 | D | D | D | D |
| GS-333 | D | C | D | C |
| GS-334 | D | D | D | C |
| GS-335 | D | D | D | C |
| GS-336 | D | D | D | D |
| GS-337 | D | D | D | C |
| GS-338 | D | D | D | B |
| GS-339 | C | A | D | B |
| GS-340 | C | D | D | D |
| GS-341 | B | A | C | B |
| GS-342 | D | D | D | D |
| GS-343 | D | D | C | B |
| GS-344 | D | D | D | D |
| GS-345 | D | D | D | D |
| GS-346 | D | D | D | D |
| GS-347 | D | D | D | B |
| GS-348 | B | A | B | A |
| GS-349 | B | B | C | B |
| GS-350 | D | D | D | A |
| GS-351 | B | B | B | A |
| GS-352 | C | C | D | C |
| GS-353 | C | B | D | B |
| GS-354 | D | D | D | D |
| GS-355 | D | B | D | C |
| GS-356 | C | C | D | C |
| GS-357 | B | B | B | B |
| GS-358 | D | D | D | D |
| GS-359 | D | D | D | D |
| GS-360 | C | C | D | B |
| GS-361 | C | B | D | B |
| GS-362 | A | A | A | A |
| GS-363 | C | C | D | D |
| GS-364 | D | D | D | D |
| GS-365 | D | D | D | C |
| GS-366 | B | A | B | A |
| GS-367 | D | D | D | D |
| GS-368 | A | A | A | A |
| GS-369 | D | D | D | D |
| GS-370 | A | A | A | A |
| GS-371 | D | D | C | C |
| GS-372 | B | A | A | A |
| GS-373 | D | D | D | D |
| GS-374 | C | D | D | C |
| GS-375 | C | B | D | B |
| GS-376 | C | B | D | B |
| GS-377 | C | B | D | A |
| GS-378 | B | A | B | A |
| GS-379 | C | B | D | B |
| GS-380 | D | D | C | C |
| GS-381 | D | C | D | B |
| GS-382 | D | D | D | B |
| GS-383 | D | C | D | D |
| GS-384 | D | D | D | D |
| GS-385 | C | B | C | B |
| GS-386 | C | B | B | A |
| GS-387 | C | B | C | A |
| GS-388 | D | D | D | D |
| GS-389 | C | B | C | B |
| GS-390 | D | C | D | C |
| GS-391 | D | D | D | C |
| GS-392 | D | C | D | D |
| GS-393 | D | D | D | D |
| GS-394 | C | B | D | B |
| GS-395 | D | B | D | B |
| GS-396 | B | A | B | A |
| GS-397 | B | A | B | A |
| GS-398 | D | D | D | D |
| GS-399 | A | A | A | A |
| GS-400 | B | B | C | B |
| GS-401 | B | A | B | A |
| GS-402 | C | B | D | B |
| GS-403 | C | B | C | B |
| GS-404 | B | A | C | A |
| GS-405 | B | B | C | A |
| GS-406 | B | A | B | A |
| GS-407 | D | C | D | B |
| GS-408 | C | B | C | B |
| GS-409 | B | B | B | A |
| GS-410 | B | A | B | A |
| GS-411 | B | A | D | A |
| GS-412 | C | B | C | B |
| GS-413 | C | C | D | B |
| GS-414 | C | B | C | B |
| GS-415 | D | D | D | D |
| GS-416 | D | D | D | C |
| GS-417 | C | B | C | B |
| GS-418 | B | A | B | A |
| GS-419 | B | B | B | A |
| GS-420 | C | B | C | B |
| GS-421 | B | A | B | A |
| GS-422 | B | A | B | A |
| GS-423 | C | B | C | B |
| GS-424 | D | D | C | B |
| GS-425 | D | B | C | B |
| GS-426 | B | A | B | A |
| GS-427 | B | A | B | A |
| GS-428 | B | A | B | A |
| GS-429 | C | A | C | B |
| GS-430 | C | B | D | B |
| GS-431 | A | A | B | A |
| GS-432 | D | B | D | B |
| GS-433 | D | D | D | D |
| GS-434 | A | A | B | A |
| GS-435 | C | D | D | C |
| GS-436 | D | C | D | C |
| GS-437 | D | D | D | C |
| GS-438 | D | D | D | C |
| GS-439 | C | B | D | B |
| GS-440 | C | C | C | C |
| GS-441 | D | B | B | C |
| GS-442 | A | A | B | A |
| GS-443 | C | C | A | D |
| GS-444 | B | A | B | A |
| GS-445 | C | C | C | C |
| GS-446 | C | B | D | B |
| GS-447 | B | A | C | B |
| GS-448 | C | B | D | C |
| GS-449 | D | D | D | D |
| GS-450 | B | B | C | B |
| GS-451 | B | B | C | B |
| GS-452 | D | D | D | D |
| GS-453 | B | B | C | B |
| GS-454 | D | C | D | C |
| GS-455 | D | D | D | D |
| GS-456 | C | B | D | B |
| GS-457 | B | B | C | B |
| GS-458 | B | A | B | A |
| GS-459 | D | D | D | D |
| GS-460 | D | D | D | D |
| GS-461 | D | D | D | D |
| GS-462 | C | B | C | B |
| GS-463 | B | A | B | A |
| GS-464 | A | A | A | A |
| GS-465 | D | C | D | B |
| GS-466 | B | B | C | B |
| GS-467 | B | A | B | B |
| GS-468 | B | B | C | B |
| GS-469 | D | D | D | D |
| GS-470 | B | A | B | A |
| GS-471 | B | B | C | B |
| GS-472 | D | D | D | D |
| GS-473 | D | D | D | D |
| GS-474 | D | D | D | D |
| GS-475 | D | D | D | D |
| GS-476 | A | A | A | A |
| GS-477 | A | A | A | A |
| GS-478 | B | A | A | A |
| GS-479 | A | A | A | A |
| GS-480 | B | A | C | A |
| GS-481 | A | A | B | A |
| GS-482 | A | A | B | A |
| GS-483 | A | A | A | A |
| GS-484 | D | C | C | C |
| GS-485 | D | D | D | D |
| GS-486 | B | A | B | A |
| GS-487 | C | B | C | A |
| GS-488 | A | A | A | A |
| GS-489 | A | A | A | A |
| GS-490 | B | A | B | A |
| GS-491 | B | A | A | A |
| GS-492 | D | D | D | D |
| GS-493 | B | A | D | A |
| GS-494 | B | A | B | A |
| GS-495 | A | A | A | A |
| GS-496 | C | B | C | B |
| GS-497 | B | A | C | A |
| GS-498 | C | B | C | B |
| GS-499 | D | C | D | C |
| GS-500 | A | A | A | A |
| GS-501 | A | A | A | A |
| GS-502 | C | B | C | B |
| GS-503 | B | A | C | B |
| GS-504 | C | B | C | B |
| GS-505 | B | B | B | A |
| GS-506 | D | D | D | C |
| GS-507 | D | C | D | B |
| GS-508 | B | B | C | B |
| GS-509 | A | A | A | A |
| GS-510 | C | A | D | A |
| GS-511 | C | B | C | A |
| GS-512 | B | B | B | A |
| GS-513 | A | A | A | A |
| GS-514 | A | A | A | A |
| GS-515 | D | B | D | C |
| GS-516 | C | B | D | B |
| GS-517 | C | B | C | B |
| GS-518 | B | A | C | A |
| GS-519 | D | D | D | D |
| GS-520 | A | A | A | A |
| GS-521 | C | B | D | B |
| GS-522 | C | B | D | A |
| GS-523 | D | C | D | C |
| GS-524 | C | C | D | B |
| GS-525 | A | A | B | A |
| GS-526 | A | A | A | A |
| GS-527 | C | C | D | C |
| GS-528 | A | A | A | A |
| GS-529 | B | A | D | A |
| GS-530 | B | A | B | A |
| GS-531 | B | A | B | A |
| GS-532 | B | A | C | A |
| GS-533 | C | B | D | D |
| GS-534 | B | A | B | A |
| GS-535 | B | B | D | A |
| GS-536 | D | C | D | B |
| GS-537 | C | C | D | B |
| GS-538 | C | B | C | B |
| GS-539 | D | C | D | C |
| GS-540 | D | B | D | B |
| GS-541 | C | A | B | A |
| GS-542 | B | A | B | A |
| GS-543 | B | A | B | A |
| GS-544 | C | A | C | A |
| GS-545 | D | C | C | B |
| GS-546 | D | C | D | B |
| GS-547 | C | B | D | B |
| GS-548 | C | A | C | A |
| GS-549 | C | B | C | A |
| GS-550 | C | B | C | B |
| GS-551 | D | C | D | B |
| GS-552 | C | B | C | A |
| GS-553 | D | B | D | B |
| GS-554 | C | B | D | B |
| GS-555 | C | B | C | A |
| GS-556 | D | D | D | D |
| GS-557 | B | A | B | A |
| GS-558 | C | C | D | B |
| GS-559 | B | B | B | A |
| GS-560 | C | B | D | B |
| GS-561 | D | C | D | B |
| GS-562 | B | B | B | A |
| GS-563 | D | B | D | C |
| GS-564 | B | A | B | A |
| GS-565 | D | C | D | B |
| GS-566 | D | C | D | C |
| GS-567 | C | B | C | A |
| GS-568 | B | A | B | A |
| GS-569 | A | A | A | A |
| GS-570 | B | A | C | A |
| GS-571 | A | A | B | A |
| GS-572 | B | A | C | A |
| GS-573 | A | A | A | A |
| GS-574 | B | A | B | A |
| GS-575 | B | A | B | A |
| GS-576 | B | A | B | A |
| GS-577 | D | D | C | C |
| GS-578 | D | C | D | C |
| GS-579 | A | A | A | A |
| GS-580 | B | A | B | A |
| GS-581 | D | B | D | A |
| GS-582 | C | B | D | B |
| GS-583 | B | A | B | A |
| GS-584 | B | B | C | A |
| GS-585 | B | A | B | A |
| GS-586 | B | A | B | A |
| GS-587 | B | A | C | A |
| GS-588 | D | D | B | C |
| GS-589 | B | A | B | A |
| GS-590 | D | C | A | C |
| GS-591 | D | C | D | C |
| GS-592 | B | A | C | B |
| GS-593 | B | A | C | B |
| GS-594 | B | A | C | A |
| GS-595 | C | B | C | B |
| GS-596 | C | C | D | C |
| GS-597 | B | D | D | C |
| GS-598 | B | A | C | B |
| GS-599 | C | B | C | C |
| GS-600 | D | D | D | D |
| GS-601 | B | D | D | B |
| GS-602 | B | A | C | B |
| GS-603 | B | A | C | B |
| GS-604 | B | A | B | A |
| GS-605 | D | D | D | D |
| GS-606 | D | D | D | D |
| GS-607 | D | D | D | D |
| GS-608 | D | D | D | D |
| GS-609 | B | A | B | A |
| GS-610 | A | A | A | A |
| GS-611 | A | A | A | A |
| GS-612 | A | A | A | A |
| GS-613 | A | A | B | A |
| GS-614 | A | A | B | A |
| GS-615 | B | A | B | A |
| GS-616 | B | A | B | A |
| GS-617 | D | D | D | D |
| GS-618 | A | A | B | A |
| GS-619 | D | D | D | D |
| GS-620 | B | A | C | A |
| GS-621 | B | A | B | A |
| GS-622 | B | A | B | A |
| GS-623 | B | A | B | A |
| GS-624 | C | B | D | B |
| GS-625 | B | A | C | B |
| GS-626 | B | B | D | B |
| GS-627 | D | D | D | D |
| GS-628 | D | B | D | B |
| GS-629 | D | C | D | C |
| GS-630 | C | A | D | A |
| GS-631 | C | B | C | B |
| GS-632 | C | C | D | C |
| GS-633 | B | A | B | B |
| GS-634 | B | A | C | B |
| GS-635 | B | A | B | A |
| GS-636 | D | D | D | D |
| GS-637 | B | A | C | B |
| GS-638 | D | D | D | D |
| GS-639 | D | D | D | D |

The IC₅₀ values (nM) of each compound were determined in MV4;11 and MOLM-13 cells with or without gilteritinib (10 nM for MV4;11; and 20 nM for MOML-13) as described in the Materials and Methods. The IC₅₀ values (nM) of each compound were determined in MV4;11 and MOLM-13 cells with or without gilteritinib (10 nM for MV4;11; and 20 nM for MOML-13) as described in Methods. A: <10 nM; 10 nM >B >=10 nM; 1 uM>C>=100 nM; D>=1 uM.

### Example 628. Pre-Clinical Studies Including a First Compound Comprising a GSPTL Degrader and a Second Compound Comprising an FLT3 Pathway Inhibitor

Compounds will be tested in a pre-clinical animal study to further assess the efficacy of some compounds described herein. For example, compounds selected from Table 1, Table 2 or Table 3, may be used in the pre-clinical animal study.

A mammalian cancer model will be used in these experiments. In short, immune compromised mice will be injected with cancer cells. The cells may include RS4;11, MV4;11, or MOLM-13 cells. The cancer model will include a GSPT1-mediated cancer. Upon injection with cancer cells, mice will receive multiple doses of the compounds with or without an FLT3 pathway inhibitor that includes gilteritinib. Cancer cells will be quantified and assessed periodically after injection into the mice. Assessments of animal and cancer cell health will be carried out. At the end of the study, animals will be sacrificed, and cancer cells and blood will be assayed for various biomarkers and cancer indices. Experiments will include measures of apoptosis, cell death, GSPT1 protein and mRNA levels.

It is expected that treatment with each of the tested compounds will be effective in treating the cancer in the mice. It is also expected that the combination treatment that includes a GSPT1-degrader compound in combination with gilteritinib will result in better cancer outcomes than a placebo control treatment or than either treatment alone. Additional tests may confirm positive effects of combination treatments with other FLT3 pathway inhibitors, or with inhibitors or modulators of other cell signaling pathways such as those described herein.

### Example 629. Clinical Trials Including a First Compound Comprising a GSPT1 Degrader and a Second Compound Comprising an FLT3 Pathway Inhibitor

Compound(s) that perform best in pre-clinical experiments will each be tested in a human clinical trial to evaluate their safety and efficacy in combination with gilteritinib in subjects with acute myeloid leukemia (AML). The compounds to be tested may include one or more compounds from Table 1, Table 2, or Table 3. The compounds are to be administered to cancer patients alone or in combination with gilteritinib. Indices of cancer growth and severity are determined overtime. It is expected that treatment with each of the compounds to be tested will be effective in treating the cancer in patients. It is also expected that the combination treatment that includes a compound to be tested in combination with gilteritinib will result in better cancer outcomes than a placebo control treatment or than either treatment alone.

### Detailed Study Description

The study will be an open-label, multi-arm, parallel multi-cohort, multicenter, study to determine the safety, tolerability, PK, and efficacy of the compounds to be tested in combination with another anti-leukemia agent used for the treatment of AML. The compounds to be tested will be given as a combination therapy to subjects with newly diagnosed (ND) or relapsed or refractory (R/R) AML.

The dose and schedule finding part (Part A) of the study will evaluate the safety, PK and PD data, and preliminary efficacy information and determine the Part B dose and schedule.

The expansion part (Part B) of the study will further evaluate the safety and efficacy of the compounds to be tested alone and combined with gilteritinib, at or below the maximum tolerated dose (MTD) in the selected cohorts in order to determine the recommended Phase 2 dose (RP2D) for subjects with AML.

Experimental: Compounds to be Tested in Combination with Gilteritinib

The compounds to be tested will each be administered intravenously per a dosing schedule in a 28-day cycle. Gilteritinib will be administered orally QD.

### Primary Outcome Measures:

1. Dose Limiting Toxicity (DLT) [Time Frame: Up to 28 days]
   Number of participants with a DLT
2. Maximum Tolerated Dose (MTD) [Time Frame: Up to 28 days.]
   The highest dose with DLT rate in Cycle 1 being lower than or close to the target level 0.3 and the toxicity probability within (0.25, 0.35) interval.
3. Adverse Events (AEs) [Time Frame: Up to 28 days after last dose of study drug.]
   An AE is any noxious, unintended, or untoward medical occurrence that may appear or worsen in a subject during the course of a study. It may be a new intercurrent illness, a worsening concomitant illness, an injury, or any concomitant impairment of the subject's health, including laboratory test values, regardless of etiology.

### Secondary Outcome Measures:

1. Complete Remission Rate (CRR), [Time Frame: Up to 3 years]
   is defined as the rate for any type of CR or CRh
2. Objective Response Rate (ORR) [Time Frame: Up to 3 years]
   is defined as the rate for all types of CRs and PR for AML.
3. Progression Free Survival (PFS) [Time Frame: Up to 3 years]
   is defined as the time from the first dose of study drug(s) to the first occurrence of relapse or progression or death from any cause
4. Overall Survival (OS) [Time Frame: Up to 3 years]
   is measured as the time from the first dose of study drug(s) to death due to any cause and will be analyzed in a manner similar to that described for PFS.
5. Duration of Remission [Time Frame: Up to 3 years]
   is measured from the time when criteria for CR/CRh/PR are first met (whichever is first recorded) until the first date at which relapse, or progressive disease is objectively documented.
6. Time to Remission [Time Frame: Up to 3 years]
   is measured from the time when criteria for CR/CRh/PR are first met (whichever is first recorded)
7. Pharmacokinetics—Cmax [Time Frame: Until last dose of the compounds to be tested in Cycle 3 (each cycle is 28 days. Dosing days for the compounds to be tested are Days 1-5 in Cycle 3)]
   observed maximum concentration in plasma
8. Pharmacokinetics—AUC24 [Time Frame: Until last dose of the compounds to be tested in Cycle 3 (each cycle is 28 days. Dosing days for the compounds to be tested are Days 1-5 in Cycle 3)]
   area under the plasma concentration time-curve from time 0 to 24 h postdose
9. Pharmacokinetics—t1/2 [Time Frame: Until last dose of the compounds to be tested in Cycle 3 (each cycle is 28 days. Dosing days for the compounds to be tested are Days 1-5 in Cycle 3)]
   terminal half life

### Inclusion Criteria:

1. Adult subject must understand and voluntarily sign an ICF prior to any study-related assessments/procedures being conducted.
2. Subject is ≥18 years of age.
3. FLT3-ITD positive relapsed or refractory AML.
4. Gilteritinib treatment naïve.
5. Subject has Eastern Cooperative Oncology Group (ECOG) performance status of 0, 1 or 2.
6. Subject must have the following screening laboratory values:
   a. Total White Blood Cell count (WBC)<25×10\hat{ }9 prior to study treatments. Treatment with hydroxyurea to achieve this level is allowed.
   b. Selected electrolytes within normal limits or correctable with supplements.
      Participant must have adequate liver function as demonstrated by: Aspartate aminotransferase (AST) and alanine aminotransferase (ALT)≤2.5×upper limit of normal (ULN) and bilirubin≤1.5×ULN
      Participant has adequate renal function as demonstrated by an estimated serum creatinine clearance of ≥60 mL/min using the Cockcroft-Gault equation.
7. Agree to follow a Pregnancy Prevention Plan (PPP) and combination agents' requirements.

### Exclusion Criteria:

1. Subject with acute promyelocytic leukemia (APL)
2. Subject has received systemic anticancer therapy (including investigational therapy) or radiotherapy<28 days or 5 half-lives, whichever is shorter, prior to the start of study treatment
3. Patients with prior autologous hematopoietic stem cell transplant (HSCT) who, in the investigator's judgment, have not fully recovered from the effects of the last transplant (e.g., transplant related side effects)
4. Prior allogeneic HSCT with either standard or reduced intensity conditioning≤6 months prior to dosing
5. Subject on systemic immunosuppressive therapy post HSCT at the time of screening, or with clinically significant graft-versus-host disease (GVHD). The use oftopical steroids for ongoing skin or ocular GVHD is permitted
6. Subject has persistent, clinically significant non-hematologic toxicities from prior therapies which have not recovered to <Grade 2
7. Subject has or is suspected of having central nervous system (CNS) leukemia. Evaluation of cerebrospinal fluid is only required if CNS involvement by leukemia is
8. Impaired cardiac function or clinically significant cardiac diseases, including any of the following:
   a. Left ventricular ejection fraction (LVEF)<45% as determined by multiple gated acquisition (MUGA) scan or echocardiogram (ECHO).
   b. Complete left bundle branch or bifascicular block.
   c. Congenital long QT syndrome.
   d. Persistent or clinically meaningful ventricular arrhythmias.
   e. QTcF>450 ms on Screening electrocardiogram (ECG)
   f. Unstable angina pectoris or myocardial infarction≤6 months prior to starting study treatments or unstable arrhythmia.
   g. Cardiovascular disability status of New York Heart Association Class 2. Class 2 is defined as cardiac disease in which patients are comfortable at rest but ordinary physical activity results in fatigue, palpitations, dyspnea, or anginal pain. suspected during screening.
9. Subject is a pregnant or lactating female

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treatment, comprising:
administering to a subject in need thereof, a first compound comprising a GSPT1 degrader and a second compound comprising an FLT3 pathway inhibitor, a RAS-RAF-MEK-ERK pathway inhibitor, or a PI3K-AKT-mTOR pathway inhibitor or activator.

2. The method of claim 1, wherein the subject has a cancer.

3. The method of any of claim 1 or 2, wherein the second compound comprises an FLT3 pathway inhibitor.

4. The method of any of claims 1-3, wherein the second compound comprises an FLT3 inhibitor.

5. The method of claim 4, wherein the FLT3 inhibitor comprises Gilteritinib, Midostaurin, Sorafenib, Sunitinib, or Lestaurtinib.

6. The method of claim 4, wherein the FLT3 inhibitor comprises Gilteritinib.

7. The method of claim 1 or 2, wherein the second compound comprises a RAS-RAF-MEK-ERK pathway inhibitor.

8. The method of claim 7, wherein the RAS-RAF-MEK-ERK pathway inhibitor comprises Vemurafenib, Dabrafenib, Encorafenib, SB590885, PLX4720, XL281, RAF265, Trametinib, Binimetinib, Cobimetinib, Selumetinib, CI-1040, or PD0325901.

9. The method of claim 1 or 2, wherein the second compound comprises a PI3K-AKT-mTOR pathway inhibitor or activator.

10. The method of claim 9, wherein the PI3K-AKT-mTOR pathway inhibitor or activator comprises Apitolisib, Idelalisib, Copanlisib, Duvelisib, MK-2206, ARQ-092, gedatolisib, Apitolisib, VQD-002, Perifosine, AZD5363, Ipatasertib, Rapamycin, temsirolimus, everolimus, ridaforolimus, Rapalogs, Sirolimus, dactolisib, BGT226, SF1126, PKI-587, NVPBE235, sapanisertib, AZD8055, and AZD2014, Wortmannin, LY294002, hibiscone C, Taselisib, Perifosine, Buparlisib, Umbralisib, PX-866, Dactolisib, CUDC-907, Voxtalisib, bisper oxovanadium, or Sarcopoterium.

11. The method of any one of claims 1-10, wherein the first compound is a compound comprising structural FORMULA 1, or a pharmaceutically acceptable salt thereof: 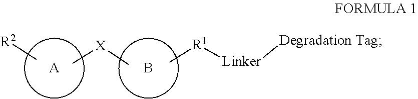
wherein
R¹ is a divalent group that is connected to a linker moiety and is absent or selected from R′—R″, R′OR″, R′SR″, R′N(R³)R″, R′OC(O)R″, R′OC(O)OR″, R′OCON(R³)R″, R′C(O)R″, R′C(O)OR″, R′CON(R³)R″, R′S(O)R″, R′S(O)₂R″, R′SO₂N(R³)R″, R′NR⁴C(O)OR″, R′NR⁴C(O)R″, R′NR⁴C(O)N(R³)R″, R′NR⁴S(O)R″, R′NR⁴S(O)₂R″, and R′NR⁴S(O)₂NR³R″, wherein
R′ and R″ are each divalent groups that are independently absent or selected from an optionally substituted C₁-C₈ alkylene, optionally substituted C₂-C₈ alkenylene, optionally substituted C₂-C₈ alkynylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R³ and R⁴ are each independently selected from an optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted 3-10 membered carbocyclyl, optionally substituted 3-10 membered heterocyclyl, optionally substituted aryl, and optionally substituted heteroaryl; or
R′ and R″, R³ and R⁴, R′ and R³, R′ and R⁴, R″ and R³, or R″ and R⁴, together with the atom to which they are attached, form a 3-20 membered carbocyclyl or 3-20 membered heterocyclyl;
R² is selected from hydrogen, halogen, oxo, CN, NO₂, OR⁵, SR⁵, N(R⁵)R⁶, OCOR⁵, OCO₂R⁵, OCON(R⁵)R⁶, COR⁵, CO₂R⁵, CON(R⁵)R⁶, SOR⁵, SO₂R⁵, SO₂N(R⁵)R⁶, NR⁷CO₂R⁵, NR⁷COR⁵, NR⁷C(O)N(R⁵)R⁶, NR⁷SOR⁵, NR⁷SO₂R⁵, NR⁷SO₂N(R⁵)R⁶, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered heterocyclylC₁-C₈alkyl, optionally substituted 3-10 membered carbocyclylC₁-C₈alkyl, optionally substituted 3-10 membered carbocyclyl, optionally substituted 3-10 membered heterocyclyl, optionally substituted aryl, and optionally substituted heteroaryl, wherein
each R⁵, R⁶, and R⁷ is independently selected from hydrogen, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, and optionally substituted heteroaryl; or
R⁵ and R⁶ or R⁵ and R⁷ together with the atom(s) to which they are attached form a 3-20 membered heterocyclyl or a 5-6 membered heteroaryl;
X is absent or is a divalent moiety selected from CR⁸R⁹, CO, CO₂, CONR⁸, NR⁸, NR⁸CO, NR⁸CO₂, NR⁸C(O)NR⁹, NR⁸SO, NR⁸SO₂, NR⁸SO₂NR⁹, O, OC(O), OCO₂, OCONR⁸, S, SO, SO₂, and SO₂NR⁸, optionally substituted C₁-C₈ alkylene, optionally substituted C₂-C₈ alkenylene, optionally substituted C₂-C₈ alkynylene, optionally substituted 3-10 membered carbocyclyl, optionally substituted 3-10 membered heterocyclyl, optionally substituted aryl, and optionally substituted heteroaryl, wherein
each R⁸ and R⁹ is independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈ alkoxy, optionally substituted C₁-C₈ alkylamino, optionally substituted 3-10 membered carbocyclyl, optionally substituted 3-10 membered heterocyclyl, optionally substituted aryl, and optionally substituted heteroaryl; or
R⁸ and R⁹ together with the atom(s) to which they are attached form a 3-20 membered carbocyclyl or 3-20 membered heterocyclyl ring; and
ring A 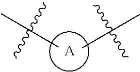
and ring B 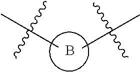
are each independently absent or selected from an optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, optionally substituted bicyclic fused aryl, optionally substituted tricyclic fused aryl, and optionally substituted heteroaryl, optionally substituted bicyclic fused heteroaryl, and optionally substituted tricyclic fused heteroaryl.

12. The method of claim 11, wherein the degradation tag is a moiety comprising structural FORMULA 5, and wherein the degradation tag is connected to the linker moiety via Z_{E}; 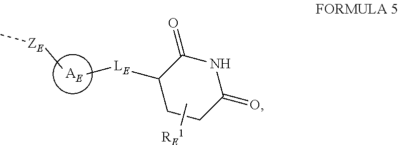
wherein
Z_{E} is a divalent group of —(R_{E}^{z})_{nE}—, wherein n_{E} is 0, 1, 2, 3, 4, 5 or 6; R_{E}^{z}, at each occurrence, is independently R_{E}^{r} or R_{E}^{w}; wherein R_{E}^{w}, at each occurrence, is absent or selected from the group consisting of —CO—, —CR_{E}⁵R_{E}⁶—, —NR_{E}⁵—, —O—, optionally substituted C₁-C₁₀ alkylene, optionally substituted C₁-C₁₀ alkenylene, optionally substituted C₁-C₁₀ alkynylene; and R_{E}, at each occurrence, is absent, or selected from the group consisting of optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl; with the proviso that —R_{E}^{z}—R_{E}^{z}— is not —O—O—; R_{E}⁵ and R_{E}⁶ at each occurrence are independently selected from the group consisting of hydrogen, halogen, oxo, hydroxy, amino, cyano, nitro, optionally substituted C₁-C₆ alkyl, optionally substituted C₁-C₈ heteroalkyl, optionally substituted C₁-C₈ haloalkyl, optionally substituted 3 to 8 membered carbocyclyl, and optionally substituted 3 to 8 membered heterocyclyl; or R_{E}S and R_{E}⁶ together with the atom(s) to which they are attached form an optionally substituted 3-8 membered carbocyclyl or heterocyclyl ring;
R_{E}¹ is selected from the group consisting of hydrogen, halogen, cyano, nitro, optionally substituted C₁-C₆ alkyl, optionally substituted C₁-C₈ heteroalkyl, optionally substituted C₁-C₈ haloalkyl, optionally substituted 3-8 membered carbocyclyl, and optionally substituted 3-8 membered heterocyclyl;
L_{E} is a divalent group selected from the group consisting of absent, -L_{E}¹-, and -L_{E}¹-L_{E}²-; wherein L_{E}¹ and L_{E}² are independently selected from the group consisting of —CO—, —O—, —CR_{E}¹⁰R_{E}¹¹— and —NR_{E}¹⁰—, with the proviso that -L_{E}¹-L_{E}²- is not —O—O—; wherein R_{E}¹⁰ and R_{E}¹¹ are independently selected from the group consisting of hydrogen, halogen, cyano, nitro, hydroxy, amino, optionally substituted C₁-C₆ alkyl, optionally substituted C₁-C₈ heteroalkyl, optionally substituted C₁-C₈ haloalkyl, optionally substituted C₁-C₆ alkoxy, and optionally substituted C₁-C₆ alkylamino; and
Ring A_{E} 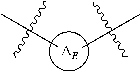
is a divalent group selected from the group consisting of FORMULA A_{E}1, A_{E}2, A_{E}3, A_{E}4 and A_{E}5: 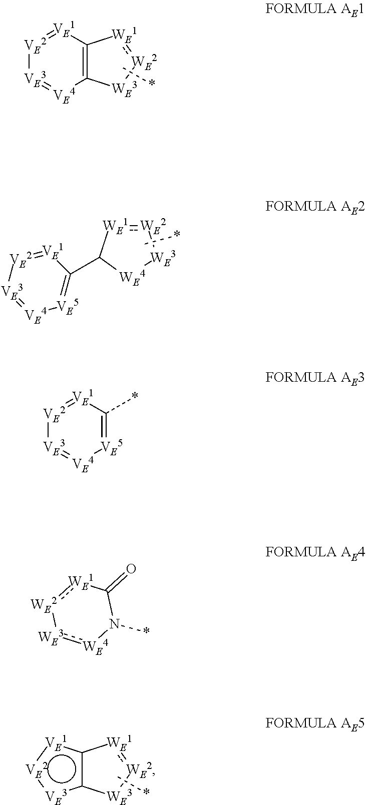
wherein
* denotes the attachment to L_{E} or to Ring A_{E} when L_{E} is absent, and Z_{E} is attached to any possible position on the Ring A_{E}, [symbol]
denotes a single bond and or a double bond;
V_{E}¹, V_{E}², V_{E}³, V_{E}⁴ and V_{E}⁵, at each occurrence, are each independently absent or selected from the group consisting of C, CR_{E}², S, N, and NR_{E}²; or V_{E}¹ and V_{E}², V_{E}² and V_{E}³, V_{E}³ and V_{E}⁴, or V_{E}⁴ and V_{E}S are optionally combined together with the atom(s) to which they attached to form a 6 membered aryl or 5, 6 or 7 membered heteroaryl ring;
R_{E}², at each occurrence, is independently selected from the group consisting of absent, hydrogen, halogen, cyano, nitro, hydroxy, amino, optionally substituted C₁-C₆ alkyl, optionally substituted C₁-C₆ alkenyl, optionally substituted C₁-C₆ alkynyl, optionally substituted C₁-C₈ heteroalkyl, optionally substituted C₁-C₈ haloalkyl, optionally substituted C₁-C₆ alkoxy, optionally substituted C₁-C₆ alkylamino, optionally substituted 3-8 membered carbocyclyl, and optionally substituted 3-8 membered heterocyclyl; or R_{E}² and another R_{E}² together with the atom(s) to which they are attached form an optionally substituted 3-8 membered carbocyclyl, optionally substituted 3-8 membered heterocyclyl, optionally substituted aryl, or optionally substituted heteroaryl;
W_{E}¹, W_{E}², W_{E}³ and W_{E}⁴ are each independently selected from the group consisting of —N═, —C≡, —CR_{E}³=, —CO—, —O—, —CR_{E}³R_{E}⁴—, —NR_{E}³—, —CR_{E}³═CR_{E}⁴—, —N═CR_{E}³—, and —N═N—; or W_{E}¹ and W_{E}², W_{E}² and W_{E}³, or W_{E}³ and W_{E} are combined together to optionally form a 6 membered aryl or a 5, 6 or 7 membered heteroaryl ring; and
R_{E}³ and R_{E}⁴, at each occurrence, are independently selected from the group consisting of absent, hydrogen, halogen, cyano, nitro, hydroxy, amino, optionally substituted C₁-C₆ alkyl, optionally substituted C₁-C₈ heteroalkyl, optionally substituted C₁-C₈ haloalkyl, optionally substituted 3 to 8 membered carbocyclyl, and optionally substituted 3 to 8 membered heterocyclyl; or R_{E}³ and R_{E}⁴, on the same atom or on the adjacent atoms, together with the atom(s) to which they are attached form an optionally substituted 3-8 membered carbocyclyl or heterocyclyl ring.

13. The method of claim 11 or 12, wherein the linker comprises a moiety of structural FORMULA 9: 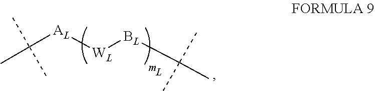
wherein
A_{L}, W_{L} and B_{L}, at each occurrence, are independently absent or a divalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}SO₂R_{L}^{e}, R_{L}^{d}SO₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ heteroalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl, wherein
R_{L}^{d} and R_{L}^{e} are each independently absent or a divalent group selected from an optionally substituted (C₁-C₈ alkylene)-R_{L}^{r}, optionally substituted R_{L}^{r}-(C₁-C₈ alkylene), optionally substituted (C₁-C₈ alkylene)-R_{L}^{r}-(C₁-C₈ alkylene), optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ heteroalkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L} is selected from an optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}¹ and R_{L}² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 3-10 membered heterocyclyl, optionally substituted aryl, and optionally substituted heteroaryl;
R_{L}^{d} and R_{L}^{e}, R_{L}¹ and R_{L}², R_{L}^{d} and R_{L}¹, R_{L}^{d} and R_{L}², R_{L}^{e} and R_{L}¹, or R_{L}^{e} and R_{L}² together with the atom(s) to which they are attached form a 3-20 membered carbocyclyl or 4-20 membered heterocyclyl ring; and
m_{L} is 1, 2, 3, 4, 5, 6, 7, 8, 9, 10, 11, 12, 13, 14, or 15.

14. The method of any one of claims 1-13, wherein the first compound is a compound disclosed in Table 1, Table 2, or Table 3, a pharmaceutically acceptable salt or solvate thereof.

15. The method of any one of claims 1-14, wherein the first compound and/or the second compound is administered to the subject as a pharmaceutical composition comprising a pharmaceutically acceptable carrier.

16. The method of any one of claims 1-14, wherein the first compound and the second compound are coadministered to the subject.

17. The method of any one of claims 1-14, wherein the first compound and the second compound are each administered separately to the subject.

18. The method of any of any one of claims 1-17 wherein the subject is a mammal.

19. The method of any one of claims 1-17, wherein the subject is a human.

20. The method of any one of claims 2-19, wherein the cancer is a GSPT1-mediated cancer.

21. A compound having the structure of FORMULA 1, or a pharmaceutically acceptable salt or solvate thereof: 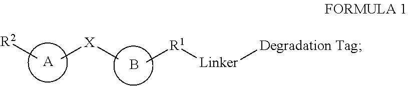
wherein
R¹ is a divalent group that is connected to a linker moiety, and is absent, or selected from R′—R″, R′OR″, R′SR″, R′N(R³)R″, R′OC(O)R″, R′OC(O)OR″, R′OCON(R³)R″, R′C(O)R″, R′C(O)OR″, R′CON(R³)R″, R′S(O)R″, R′S(O)₂R″, R′SO₂N(R³)R″, R′NR⁴C(O)OR″, R′NR⁴C(O)R″, R′NR⁴C(O)N(R³)R″, R′NR⁴S(O)R″, R′NR⁴S(O)₂R″, and R′NR⁴S(O)₂NR³R″, wherein
R′ and R″ are each divalent groups that are independently absent or selected from an optionally substituted C₁-C₈ alkylene, optionally substituted C₂-C₈ alkenylene, optionally substituted C₂-C₈ alkynylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R³ and R⁴ are independently selected from an optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted 3-10 membered carbocyclyl, optionally substituted 3-10 membered heterocyclyl, optionally substituted aryl, and optionally substituted heteroaryl; or
R′ and R″, R³ and R⁴, R′ and R³, R′ and R⁴, R″ and R³, or R″ and R⁴, together with the atom(s) to which they are attached, form a 3-20 membered carbocyclyl or 3-20 membered heterocyclyl ring;
R² is selected from hydrogen, halogen, oxo, CN, NO₂, OR⁵, SR⁵, N(R⁵)R⁶, OCOR⁵, OCO₂R⁵, OCON(R⁵)R⁶, COR⁵, CO₂R⁵, CON(R⁵)R⁶, SOR⁵, SO₂R⁵, SO₂N(R⁵)R⁶, NR⁷CO₂R⁵, NR⁷COR⁵, NR⁷C(O)N(R⁵)R⁶, NR⁷SOR⁵, NR⁷SO₂R⁵, NR⁷SO₂N(R⁵)R⁶, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered heterocyclylC₁-C₈alkyl, optionally substituted 3-10 membered carbocyclylC₁-C₈alkyl, optionally substituted 3-10 membered carbocyclyl, optionally substituted 3-10 membered heterocyclyl, optionally substituted aryl, and optionally substituted heteroaryl, wherein
each R⁵, R⁶, and R⁷ is independently selected from hydrogen, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, and optionally substituted heteroaryl; or
R⁵ and R⁶ or R⁵ and R⁷ together with the atom to which they are attached form a 3-20 membered heterocyclyl, or 5-6 membered heteroaryl;
X is absent or is selected from a divalent group comprising CR⁸R⁹, CO, CO₂, CONR⁸, NR⁸, NR⁸CO, NR⁸CO₂, NR⁸C(O)NR⁹, NR⁸SO, NR⁸SO₂, NR⁸SO₂NR⁹, O, OC(O), OCO₂, OCONR⁸, S, SO, SO₂, and SO₂NR⁸, optionally substituted C₁-C₈ alkylene, optionally substituted C₂-C₈ alkenylene, optionally substituted C₂-C₈ alkynylene, optionally substituted 3-10 membered carbocyclyl, optionally substituted 3-10 membered heterocyclyl, optionally substituted aryl, and optionally substituted heteroaryl, wherein
each R⁸ and R⁹ is independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₁-C₈ haloalkyl, optionally substituted C₁-C₈ alkoxy, optionally substituted C₁-C₈ alkylamino, optionally substituted 3-10 membered carbocyclyl, optionally substituted 3-10 membered heterocyclyl, optionally substituted aryl, and optionally substituted heteroaryl; or
R⁸ and R⁹ together with the atom(s) to which they are attached form a 3-20 membered carbocyclyl or 3-20 membered heterocyclyl ring; and
ring A 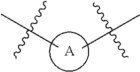
and ring B 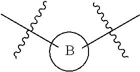
are each independently absent or selected from an optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, optionally substituted bicyclic fused aryl, optionally substituted tricyclic fused aryl, and optionally substituted heteroaryl, optionally substituted bicyclic fused heteroaryl, and optionally substituted tricyclic fused heteroaryl.

22. The compound of claim 21, wherein the degradation tag is a moiety comprising structural FORMULA 5, and wherein the degradation tag is connected to the linker moiety through Z_{E}; 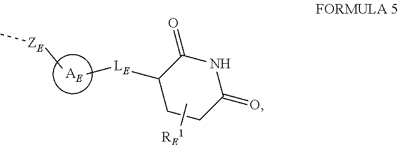
wherein
Z_{E} is a divalent group —(R_{E}^{z})_{nE}—; wherein n_{E} is 0, 1, 2, 3, 4, 5 or 6; R_{E}^{z}, at each occurrence, is independently R_{E}^{r}, or R_{E}^{w}; wherein R_{E}^{w}, at each occurrence, is absent or selected from the group consisting of —C(O)—, —CR_{E}⁵R_{E}⁶—, —NR_{E}⁵—, —O—, optionally substituted C₁-C₁₀ alkylene, optionally substituted C₁-C₁₀ alkenylene, optionally substituted C₁-C₁₀ alkynylene; and R_{E}^{r}, at each occurrence, is absent, or selected from the group consisting of optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl; with the proviso that —R_{E}^{z}—R_{E}^{z}— is not —O—O—; R_{E}⁵ and R_{E}⁶ at each occurrence are independently selected from the group comprising hydrogen, halogen, oxo, hydroxy, amino, cyano, nitro, optionally substituted C₁-C₆ alkyl, optionally substituted C₁-C₈ heteroalkyl, optionally substituted C₁-C₈ haloalkyl, optionally substituted 3 to 8 membered carbocyclyl, and optionally substituted 3 to 8 membered heterocyclyl; or R_{E}⁵ and R_{E}⁶ together with the atom(s) to which they are attached form an optionally substituted 3-8 membered carbocyclyl or heterocyclyl ring;
R_{E}¹ is selected from the group consisting of hydrogen, halogen, cyano, nitro, optionally substituted C₁-C₆ alkyl, optionally substituted C₁-C₈ heteroalkyl, optionally substituted C₁-C₈ haloalkyl, optionally substituted 3-8 membered carbocyclyl, and optionally substituted 3-8 membered heterocyclyl;
L_{E} is a divalent group selected from the group consisting of absent, -L_{E}¹-, and -L_{E}¹-L_{E}²-; wherein L_{E}¹ and L_{E}² are independently selected from the group consisting of —CO—, —O—, —CR_{E}¹⁰R_{E}¹¹— and —NR_{E}¹⁰—, with the proviso that -L_{E}¹-L_{E}²- is not —O—O—; wherein R_{E}¹⁰ and R_{E}¹¹ are independently selected from the group consisting of hydrogen, halogen, cyano, nitro, hydroxy, amino, optionally substituted C₁-C₆ alkyl, optionally substituted C₁-C₈ heteroalkyl, optionally substituted C₁-C₈ haloalkyl, optionally substituted C₁-C₆ alkoxy, and optionally substituted C₁-C₆ alkylamino; and
Ring A_{E} 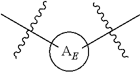
is a divalent group selected from the group consisting of FORMULA A_{E}1, A_{E}2, A_{E}3, A_{E}4 and A_{E}5: 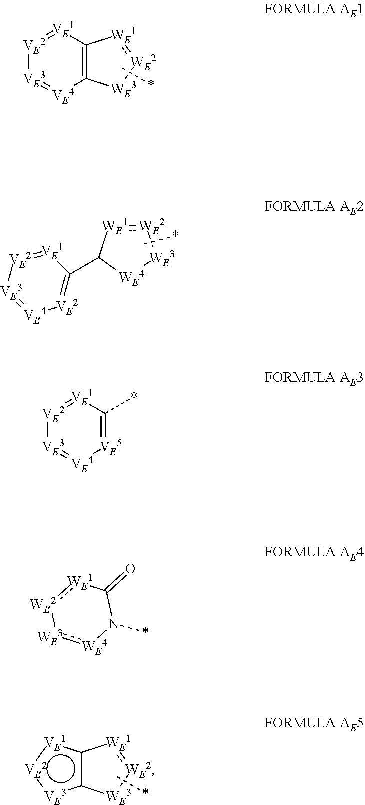
wherein
* denotes the attachment to L_{E} or to Ring A_{E} when L_{E} is absent, and Z_{E} is attached to any possible position on the Ring A_{E}, [symbol]
denotes a single bond or a double bond;
V_{E}¹, V_{E}², V_{E}³, V_{E}⁴ and V_{E}⁵, at each occurrence, are independently selected from the group consisting of absent, C, CR_{E}², S, N, and NR_{E}²; or V_{E}¹ and V_{E}^{Z}, V_{E}² and V_{E}³, V_{E}³ and V_{E}⁴, or V_{E}⁴ and V_{E}⁵ are combined together with atom(s) to which they are attached to optionally form a 6 membered aryl or 5, 6 or 7 membered heteroaryl ring;
R_{E}², at each occurrence, is independently selected from the group consisting of absent, hydrogen, halogen, cyano, nitro, hydroxy, amino, optionally substituted C₁-C₆ alkyl, optionally substituted C₁-C₆ alkenyl, optionally substituted C₁-C₆ alkynyl, optionally substituted C₁-C₈ heteroalkyl, optionally substituted C₁-C₈ haloalkyl, optionally substituted C₁-C₆ alkoxy, optionally substituted C₁-C₆ alkylamino, optionally substituted 3-8 membered carbocyclyl, and optionally substituted 3-8 membered heterocyclyl; or R_{E}² and another R_{E}² together with the atom(s) to which they are connected form optionally substituted 3-8 membered carbocyclyl, optionally substituted 3-8 membered heterocyclyl, optionally substituted aryl, and optionally substituted heteroaryl;
W_{E}¹, W_{E}², W_{E}³ and W_{E}⁴ are each independently selected from the group consisting of —N═, —C≡, —CR_{E}³=, —CO—, —O—, —CR_{E}³R_{E}⁴—, —NR_{E}³—, —CR_{E}³═CR_{E}⁴—, —N═CR_{E}³—, and —N═N—; or W_{E}¹ and W_{E}², W_{E}² and W_{E}³, or W_{E}3 and W_{E}⁴ are combined together with the atom(s) to which they are attached to optionally form a 6 membered aryl or 5, 6 or 7 membered heteroaryl ring; and
R_{E}³ and R_{E}⁴, at each occurrence, are independently selected from the group consisting of absent, hydrogen, halogen, cyano, nitro, hydroxy, amino, optionally substituted C₁-C₆ alkyl, optionally substituted C₁-C₈ heteroalkyl, optionally substituted C₁-C₈ haloalkyl, optionally substituted 3 to 8 membered carbocyclyl, and optionally substituted 3 to 8 membered heterocyclyl; or R_{E}³ and R_{E}⁴, on the same atom or on the adjacent atoms, together with the atom(s) to which they are attached form an optionally substituted 3-8 membered carbocyclyl or heterocyclyl ring.

23. The compound of claim 22, wherein the moiety of Formula 5 has the structure of Formula 5-1: 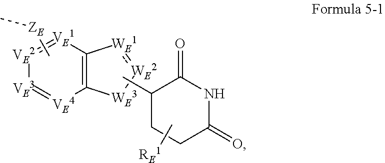
wherein, [symbol]
denotes a single bond or a double bond;
R_{E}¹ is hydrogen;
Z_{E} is absent, —CH₂—, —NH—, or —O—;
V_{E}¹, V_{E}², V_{E}³, and V_{E}⁴ are each independently C, CR_{E}², or N;
W_{E}¹, W_{E}², and W_{E}³ are each independently selected from the group consisting of —C(O)—, —N—, —NR_{E}³—, or —CR_{E}³R_{E}⁴—;
each R_{E}² is independently hydrogen; and
R_{E}³ and R_{E}⁴ are each independently selected from the group consisting of hydrogen, halogen, cyano, nitro, hydroxy, amino, or optionally substituted C₁-C₆ alkyl; or R_{E}³ and R_{E}⁴ together with the atom(s) to which they are attached form an optionally substituted 3-6 membered carbocyclyl or 3-6 membered heterocyclyl ring.

24. The compound of any one of claims 21-23, wherein the linker moiety comprises structural FORMULA 9: 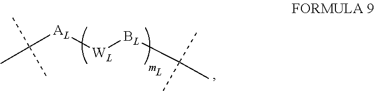
wherein
A_{L}, W_{L} and B_{L}, at each occurrence, are independently absent or a divalent moiety selected from R_{L}^{d}—R_{L}^{e}, R_{L}^{d}COR_{L}^{e}, R_{L}^{d}CO₂R_{L}^{e}, R_{L}^{d}C(O)N(R_{L}¹)R_{L}^{e}, R_{L}^{d}C(S)N(R_{L}¹)R_{L}^{e}, R_{L}^{d}OR_{L}^{e}, R_{L}^{d}SR_{L}^{e}, R_{L}^{d}SOR_{L}^{e}, R_{L}^{d}O₂R_{L}^{e}, R_{L}^{d}O₂N(R_{L}¹)R_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, R_{L}^{d}N(R_{L}¹)C(S)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ heteroalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl, wherein
R_{L}^{d} and R_{L}^{e} are each independently absent or a divalent group selected from an optionally substituted (C₁-C₈ alkylene)-R_{L}^{r}, optionally substituted R_{L}^{r}-(C₁-C₈ alkylene), optionally substituted (C₁-C₈ alkylene)-R_{L}^{r}-(C₁-C₈ alkylene), an optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ heteroalkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}^{r} is selected from optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}¹ and R_{L}² are each independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 3-10 membered heterocyclyl, optionally substituted aryl, and optionally substituted heteroaryl; or
R_{L}^{d} and R_{L}^{e}, R_{L}¹ and R_{L}², R_{L}^{d} and R_{L}¹, R_{L}^{d} and R_{L}², R_{L}^{e} and R_{L}¹, or R_{L}^{e} and R_{L}² together with the atom(s) to which they are attached optionally form a 3-20 membered carbocyclyl or 4-20 membered heterocyclyl ring; and
m_{L} is 1, 2, 3, 4, 5, 6, 7, 8, 9, 10, 11, 12, 13, 14, or 15.

25. The compound of claim 24, wherein
A_{L} is an optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, or optionally substituted 3-10 membered heterocyclyl;
W_{L} and B_{L}, at each occurrence, are independently absent or a divalent moiety selected from R_{L}^{d}—R_{L}^{e}, R_{L}^{d}C(O)R_{L}^{e}, R_{L}^{d}CO₂R_{L}^{e}, R_{L}^{d}C(O)N(R_{L}¹)R_{L}^{e}, R_{L}^{d}OR_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{e}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ heteroalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl;
R_{L}^{d} and R_{L}^{e} are each independently absent or a divalent group selected from an optionally substituted (C₁-C₈ alkylene)-R_{L}^{r}, optionally substituted R_{L}^{r}-(C₁-C₈ alkylene), optionally substituted (C₁-C₈ alkylene)-R_{L}^{r}-(C₁-C₈ alkylene), optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ heteroalkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}^{r} is selected from an optionally substituted aryl, and optionally substituted heteroaryl; and
m_{L} is 1, 2, or 3.

26. The compound of claim 25, wherein A_{L} is an optionally substituted 5-13 membered spiro carbocyclyl, optionally substituted 5-13 membered spiro heterocyclyl, optionally substituted 3-10 membered carbocyclyl, or optionally substituted 3-10 membered heterocyclyl.

27. The compound of any one of claims 21-26, wherein:
ring A is absent, or an optionally substituted 3-10 membered carbocyclyl, optionally substituted 3-10 membered heterocyclyl, optionally substituted aryl, optionally substituted bicyclic aryl, optionally substituted heteroaryl, or optionally substituted bicyclic heteroaryl; and
ring B is absent, or an optionally substituted 3-10 membered carbocyclyl, optionally substituted 3-10 membered heterocyclyl, optionally substituted aryl, or optionally substituted heteroaryl.

28. The compound of any one of claims 21-27, wherein
ring B is absent or a is a moiety comprising structural Formula B-1: 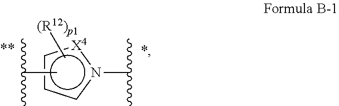
wherein,
X⁴ is CR¹² or N;
each R¹² is independently hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl; or
two R¹² on adjacent carbon atoms join together to form an optionally substituted optionally substituted 3-7 membered carbocyclyl, optionally substituted 4-7 membered heterocyclyl, optionally substituted 6 membered aryl, or optionally substituted 5-6 membered heteroaryl ring;
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or
R⁵ and R⁶ together with the atom(s) to which they are attached optionally form an optionally substituted 4-7 membered heterocyclyl, or optionally substituted 5-6 membered heteroaryl ring;
p₁ is 0, 1, or 2;
* denotes the connection to the linker moiety; and
* denotes the connection to X and/or ring A; and
ring A is a moiety having the structure of Formula A-1: 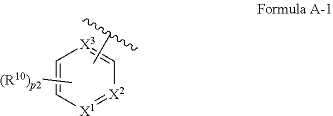
wherein,
X¹, X², and X³ are each independently CR¹⁰ or N;
each R¹⁰ is independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino; or
two R¹⁰ on adjacent carbon atoms join together to form an optionally substituted 3-7 membered carbocyclyl, optionally substituted 4-7 membered heterocyclyl, optionally substituted 6 membered aryl, or optionally substituted 5-6 membered heteroaryl ring;
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or
R⁵ and R together with the atom(s) to which they are attached optionally form an optionally substituted 4-7 membered heterocyclyl, or optionally substituted 5-6 membered heteroaryl ring; and
p₂ is 0, 1, 2, 3, or 4.

29. The compound of claim 29, wherein ring A is a moiety comprising structural Formula A-2, Formula A-3, or Formula A-4: 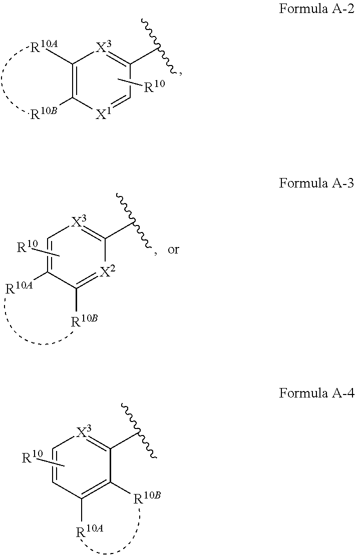
wherein,
X¹ is CR¹⁰ or N;
X² is CR¹⁰ or N;
X³ is CR¹⁰ or N;
R^{10A} and R^{10B} are each independently R¹⁰; or
R^{10A} and R^{10B} with the atoms to which they are attached join together to form an optionally substituted 6 membered aryl or 5-6 membered heteroaryl ring;
each R¹⁰ is independently H, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino; and
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or
R⁵ and R⁶ together with the atom(s) to which they are attached optionally form an optionally substituted 4-7 membered heterocyclyl, or optionally substituted 5-6 membered heteroaryl ring.

30. The compound of claim 29, wherein ring A comprises structural Formula A-5: 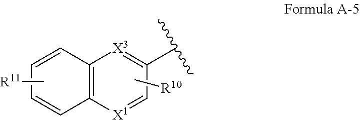
wherein,
R¹¹ is hydrogen, halogen, CN, NO₂, OR⁵, SR⁵, N(R⁵)R⁶, C(O)R⁵, C(O)OR⁵, C(O)N(R⁵)R⁶, SO₂R⁵, SO₂N(R⁵)R⁶, NR′C(O)OR⁵, NR⁷C(O)R⁵, NR⁷C(O)N(R⁵)R⁶, NR⁷SOR⁵, NR⁷SO₂R⁵, NR⁷SO₂N(R⁵)R⁶ optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈ alkoxy, or optionally substituted C₁-C₈ alkylamino, optionally substituted 3-10 membered carbocyclyl, or optionally substituted 3-10 membered heterocyclyl.

31. The compound of claim 29, wherein ring A comprises structural Formula A-6: 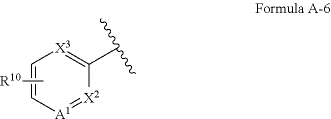
wherein, at least one of X¹, X², or X³ is N.

32. A compound comprising structural FORMULA 1, or a pharmaceutically acceptable salt or solvate thereof: 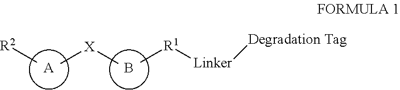
wherein,
ring A is absent, or an optionally substituted 3-10 membered carbocyclyl, optionally substituted 3-10 membered heterocyclyl, optionally substituted aryl, optionally substituted bicyclic aryl, optionally substituted heteroaryl, or optionally substituted bicyclic heteroaryl;
ring B is absent, or an optionally substituted 3-10 membered carbocyclyl, optionally substituted 3-10 membered heterocyclyl, optionally substituted aryl, or optionally substituted heteroaryl;
R¹ is absent;
R² is hydrogen, halogen, CN, NO₂, OR⁵, SR⁵, N(R⁵)R⁶, C(O)R⁵, C(O)OR⁵, C(O)N(R⁵)R⁶, SO₂R⁵, SO₂N(R⁵)R⁶, NR⁷C(O)OR⁵, NR⁷C(O)R⁵, NR⁷C(O)N(R⁵)R⁶, NR⁷SOR⁵, NR⁷SO₂R⁵, NR⁷SO₂N(R⁵)R⁶ optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ alkoxy, or optionally substituted C₁-C₈ alkylamino, optionally substituted 3-10 membered carbocyclyl, or optionally substituted 3-10 membered heterocyclyl;
X is absent, —O—, or NR⁵;
each R⁵, R⁶ and R⁷ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or
R⁵ and R⁶ together with the atom(s) to which they are attached optionally form a 3-20 membered heterocyclyl or 5-6 membered heteroaryl ring;
R⁸ is hydrogen or C₁-C₈ alkyl; and
Linker is a linking moiety connecting the degradation tag to ring B.

33. The compound of claim 32, wherein the degradation tag comprises structural Formula 5-1: 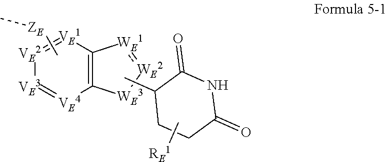
wherein, [symbol]
denotes a single bond or a double bond;
R_{E}¹ is hydrogen;
Z_{E} is absent, —CH₂—, —NH—, or —O—;
V_{E}¹, V_{E}², V_{E}³, and V_{E}⁴ are each independently C, CR_{E}², or N;
W_{E}¹, W_{E}², and W_{E}³ are each independently selected from the group consisting of —C(O)—, —N—, —NR_{E}³—, or —CR_{E}³R_{E}⁴—,
each CR_{E}² is independently hydrogen; and
R_{E}³ and R_{E}⁴ are each independently selected from the group consisting of hydrogen, halogen, cyano, nitro, hydroxy, amino, or optionally substituted C₁-C₆ alkyl.

34. The compound of claim 33, wherein:
W_{E}¹ and W_{E}³ are each independently C(O) or CH₂; and
W_{E}² is N.

35. The compound of claim 33 or 34, wherein the degradation tag has the structure of

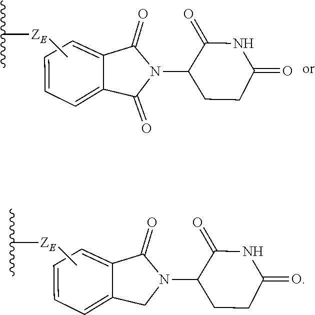

36. The compound of any one of claims 32-35, wherein:
ring A is an optionally substituted 3-10 membered carbocyclyl, optionally substituted 3-10 membered heterocyclyl, optionally substituted aryl, optionally substituted bicyclic aryl, optionally substituted heteroaryl, or optionally substituted bicyclic heteroaryl; and
ring B is absent or an optionally substituted heteroaryl.

37. The compound of any one of claims 32-35, wherein:
ring B is absent or a is a moiety comprising structural Formula B-1: 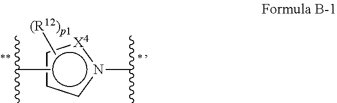
wherein,
X⁴ is CR¹² or N;
each R¹² is independently hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted C₁-C₈ heteroalkyl, optionally substituted 3-10 membered carbocyclyl, or optionally substituted 3-10 membered heterocyclyl; or
two R¹² on adjacent carbon atoms join together to form an optionally substituted optionally substituted 3-7 membered carbocyclyl, optionally substituted 4-7 membered heterocyclyl, optionally substituted 6 membered aryl, or optionally substituted 5-6 membered heteroaryl ring;
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or
R⁵ and R⁶ together with the atom(s) to which they are attached optionally form an optionally substituted 4-7 membered heterocyclyl, or optionally substituted 5-6 membered heteroaryl ring;
p₁ is 0, 1, or 2;
* denotes the connection to the linker moiety; and
* denotes the connection to X and/or ring A; and
ring A is a moiety comprising structural Formula A-1: 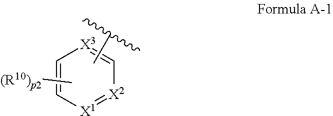
wherein,
X¹, X², and X³ are each independently CR¹⁰ or N;
each R¹⁰ is independently hydrogen, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino; or
two R¹⁰ on adjacent carbon atoms join together to form an optionally substituted 3-7 membered carbocyclyl, optionally substituted 4-7 membered heterocyclyl, optionally substituted 6 membered aryl or optionally substituted 5-6 membered heteroaryl ring;
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or
R⁵ and R⁶ together with the atom(s) to which they are attached optionally form an optionally substituted 4-7 membered heterocyclyl, or optionally substituted 5-6 membered heteroaryl ring; and
p₂ is 0, 1, 2, 3, or 4.

38. The compound of claim 37, wherein ring A is a moiety comprising structural Formula A-2, Formula A-3, or Formula A-4: 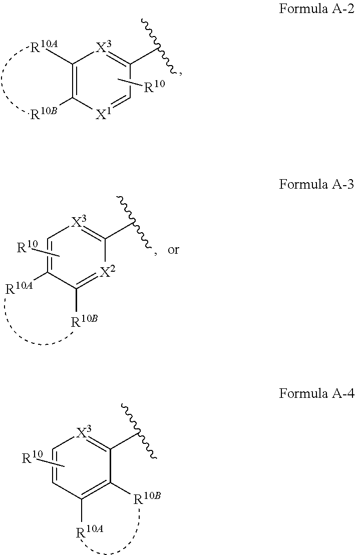
wherein,
X¹ is CR¹⁰ or N;
X² is CR¹⁰ or N;
X³ is CR¹⁰ or N;
R^{10A} and R^{10B} are each independently R¹⁰; or
R^{10A} and R^{10B} with the atoms to which they are attached together to form an optionally substituted 6 membered aryl or 5-6 membered heteroaryl ring;
each R¹⁰ is independently H, halogen, CN, NO₂, OR⁵, SR⁵, N(R⁵)R⁶, C(O)R⁵, C(O)OR⁵, C(O)N(R⁵)R⁶, optionally substituted C₁-C₈ alkyl, optionally substituted C₁-C₈ haloalkyl, optionally substituted C₁-C₈ heteroalkyl, optionally substituted C₁-C₈ alkoxy, or optionally substituted C₁-C₈ alkylamino; and
each R⁵ and R⁶ is independently hydrogen, optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 3-10 membered heterocyclylC₁-C₈alkyl, optionally substituted 3-10 membered carbocyclyl, optionally substituted 3-10 membered heterocyclyl, optionally substituted aryl, or optionally substituted heteroaryl; or
R⁵ and R⁶ together with the atom(s) to which they are connected optionally form an optionally substituted 4-7 membered heterocyclyl, or optionally substituted 5-6 membered heteroaryl ring.

39. The compound of claim 38, wherein ring A comprises structural Formula A-5: 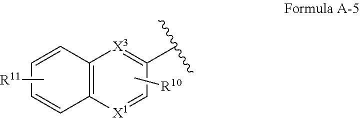
wherein,
R¹¹ is hydrogen, halogen, CN, NO₂, OR⁵, SR⁵, N(R⁵)R⁶, C(O)R⁵, C(O)OR⁵, C(O)N(R⁵)R⁶, SO₂R⁵, SO₂N(R⁵)R⁶, NR⁷C(O)OR⁵, NR⁷C(O)R⁵, NR⁷C(O)N(R⁵)R⁶, NR⁷SOR⁵, NR⁷SO₂R⁵, NR⁷SO₂N(R⁵)R⁶ optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl.

40. The compound of claim 38, wherein ring A comprises structural Formula A-7: 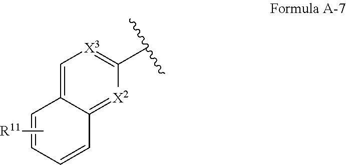
wherein,
R¹¹ is hydrogen, halogen, CN, NO₂, OR⁵, SR⁵, N(R⁵)R⁶, C(O)R⁵, C(O)OR⁵, C(O)N(R⁵)R⁶, SO₂R⁵, SO₂N(R⁵)R⁶, NR⁷C(O)OR⁵, NR⁷C(O)R⁵, NR⁷C(O)N(R⁵)R⁶, NR⁷SOR⁵, NR⁷SO₂R⁵, NR⁷SO₂N(R⁵)R⁶ optionally substituted C₁-C₈ alkyl, optionally substituted C₂-C₈ alkenyl, optionally substituted C₂-C₈ alkynyl, optionally substituted C₁-C₈ haloalkyl, optionally substituted C₁-C₈ heteroalkyl, optionally substituted C₁-C₈ alkoxy, optionally substituted C₁-C₈ alkylamino, optionally substituted 3-10 membered carbocyclyl, or optionally substituted 3-10 membered heterocyclyl.

41. The compound of claim 37, wherein ring A comprises structural Formula A-6: 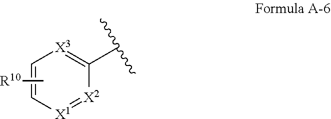
wherein, at least one of X¹, X², or X³ is N.

42. The compound of any one of claims 37-41, claim wherein X³ is N.

43. The compound of any one of claims 37-41, wherein each R¹⁰ is independently hydrogen, halogen, CN, NO₂, OR⁵, N(R⁵)R⁶, C(O)R⁵, C(O)OR⁵, optionally substituted C₁-C₈ alkyl, optionally substituted C₁-C₈ alkoxy, optionally substituted 3-10 membered carbocyclyl, or optionally substituted 3-10 membered heterocyclyl.

44. The compound of claim 39 or 40, wherein R″ is hydrogen, halogen, CN, NO₂, OR⁵, N(R⁵)R⁶, C(O)R⁵, C(O)OR⁵, C(O)N(R⁵)R⁶, optionally substituted C₁-C₈ alkyl, optionally substituted C₂-C₈ alkynyl, optionally substituted C₁-C₈ alkoxy, optionally substituted 3-10 membered carbocyclyl, or optionally substituted 3-10 membered heterocyclyl.

45. The compound of any one of claims 37-44, wherein ring B comprises structural Formula B-2:

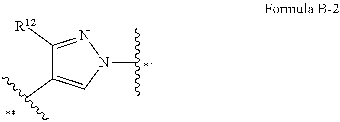

46. The compound of claim 45, wherein R″ is hydrogen, halogen, CN, OR⁵, N(R⁵)R⁶, C(O)R⁵, C(O)OR⁵, C(O)N(R⁵)R⁶, optionally substituted C₁-C₈ alkyl, optionally substituted C₁-C₈ alkoxy, optionally substituted C₁-C₈ alkylamino, optionally substituted C₁-C₈ haloalkyl, optionally substituted 3-10 membered carbocyclyl, or optionally substituted 3-10 membered heterocyclyl.

47. The compound of claim 46, wherein R¹² is hydrogen, halogen, CN, OH, CH₃, —OCH₃, CF₃, CF₂, cyclopropyl, cyclobutyl, or cyclopentyl.

48. The compound of any one of claims 32-47, wherein X is absent.

49. The compound of any one of claims 32-48, wherein the linker comprises structural Formula 9: 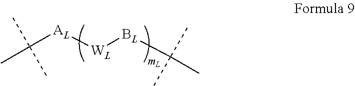
wherein,
A_{L} is an optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, or optionally substituted 3-10 membered heterocyclyl;
W_{L} and B_{L}, at each occurrence, are independently absent or a divalent moiety selected from R_{L}^{d}—R_{L}^{e}, R_{L}^{d}C(O)R_{L}^{e}, R_{L}^{d}CO₂R_{L}^{e}, R_{L}^{d}C(O)N(R_{L}¹)R_{L}^{e}, R_{L}^{d}OR_{L}^{e}, R_{L}^{d}N(R_{L}¹)R_{L}^{e}, R_{L}^{d}N(R_{L}¹)COR_{L}^{e}, R_{L}^{d}N(R_{L}¹)CON(R_{L}²)R_{L}^{c}, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ heteroalkylene, optionally substituted C₁-C₈ hydroxyalkylene, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted 3-10 membered carbocyclyl, optionally substituted 3-10 membered heterocyclyl, optionally substituted aryl, and optionally substituted heteroaryl;
R_{L}^{d} and R_{L}^{e} are each independently absent or divalent group selected from an optionally substituted (C₁-C₈ alkylene)-R_{L}^{r}, optionally substituted R_{L}^{r}-(C₁-C₈ alkylene), optionally substituted (C₁-C₈ alkylene)-R_{L}^{r}-(C₁-C₈ alkylene), optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ heteroalkylene, optionally substituted C₁-C₈ haloalkylene, optionally substituted 3-10 membered carbocyclyl, optionally substituted 3-10 membered heterocyclyl, optionally substituted 4-13 membered fused carbocyclyl, optionally substituted 5-13 membered fused heterocyclyl, optionally substituted 5-13 membered bridged carbocyclyl, optionally substituted 5-13 membered bridged heterocyclyl, optionally substituted 5-13 membered spiro carbocyclyl, optionally substituted 5-13 membered spiro heterocyclyl, optionally substituted aryl, and optionally substituted heteroaryl;
R_{L}^{r} is selected from an optionally substituted aryl or optionally substituted heteroaryl; and
m_{L} is 1, 2, or 3.

50. The compound of claim 49, wherein
A_{L} is an optionally substituted 3-10 membered carbocyclyl or optionally substituted 3-10 membered heterocyclyl;
W_{L} and B_{L}, at each occurrence, are independently absent or a divalent moiety selected from R_{L}^{d}—R_{L}^{e}, R_{L}^{d}C(O)R_{L}^{e}, R_{L}^{d}CO₂R_{L}^{e}, R_{L}^{d}C(O)N(R_{L}¹)R_{L}^{e}, R_{L}^{d}OR_{L}^{e}, R_{L}^{d}N(R_{L}¹)R_{L}^{e}, and R_{L}^{d}N(R_{L}¹)COR_{L}^{e};
R_{L}^{d} and R_{L}^{e} are each independently absent or selected from an optionally substituted (C₁-C₈ alkylene)-R_{L}¹, optionally substituted R_{L}^{r}-(C₁-C₈ alkylene), optionally substituted (C₁-C₈ alkylene)-R_{L}^{r}-(C₁-C₈ alkylene), optionally substituted C₁-C₈ alkyl, optionally substituted aryl and optionally substituted heteroaryl; and
R_{L}^{r} is selected from an optionally substituted aryl, and optionally substituted heteroaryl.

51. The compound of any one of claims 32-50, wherein the linker is -(3-10 membered carbocyclyl)-(C₁-C₈ alkylene)- or -(3-10 membered heterocyclyl)-(C₁-C₈ alkylene)-.

52. The compound of any of claims 21-51, wherein the compound is selected from Table 1, Table 2, or Table 3, wherein the compound is not CC-90009 and gilteritinib, or a pharmaceutically acceptable salt or solvate thereof.

53. The compound of any one of claims 21-51, wherein the compound is GS-025, GS-031, GS-034, GS-035, GS-036, GS-043, GS-044, GS-045, GS-048, GS-049, GS-050, GS-051, GS-052, GS-053, GS-054, GS-060, GS-066, GS-068, GS-069, GS-070, GS-076, GS-077, GS-078, GS-087, GS-090, GS-093, GS-096, GS-097, GS-098, GS-101, GS-102, GS-105, GS-110, GS-113, GS-115, GS-116, GS-118, GS-120, GS-122, GS-123, GS-132, GS-134, GS-145, GS-150, GS-151, GS-156, GS-163, GS-166, GS-167, GS-171, GS-173, GS-174, GS-175, GS-181, GS-182, GS-188, GS-191, GS-194, GS-195, GS-196, GS-198, GS-199, GS-202, GS-204, GS-205, GS-207, GS-210, GS-211, GS-213, GS-215, GS-217, GS-221, GS-223, GS-224, GS-227, GS-228, GS-229, GS-230, GS-231, GS-240, GS-243, GS-247, GS-253, GS-254, GS-255, GS-256, GS-257, GS-259, GS-261, GS-262, GS-264, GS-266, GS-267, GS-272, GS-276, GS-277, GS-278, GS-280, GS-281, GS-282, GS-284, GS-285, GS-286, GS-287, GS-289, GS-299, GS-305, GS-308, GS-309, GS-311, GS-314, GS-315, GS-323, GS-339, GS-341, GS-348, GS-350, GS-351, GS-362, GS-366, GS-368, GS-370, GS-372, GS-377, GS-378, GS-386, GS-387, GS-396, GS-397, GS-399, GS-401, GS-404, GS-405, GS-406, GS-409, GS-410, GS-411, GS-418, GS-419, GS-421, GS-422, GS-426, GS-427, GS-428, GS-429, GS-431, GS-434, GS-442, GS-444, GS-447, GS-458, GS-463, GS-464, GS-467, GS-470, GS-476, GS-477, GS-478, GS-479, GS-480, GS-481, GS-482, GS-483, GS-486, GS-487, GS-488, GS-489, GS-490, GS-491, GS-493, GS-494, GS-495, GS-497, GS-500, GS-501, GS-503, GS-505, GS-509, GS-510, GS-511, GS-512, GS-513, GS-514, GS-518, GS-520, GS-522, GS-525, GS-526, GS-528, GS-529, GS-530, GS-531, GS-532, GS-534, GS-535, GS-541, GS-542, GS-543, GS-544, GS-548, GS-549, GS-552, GS-555, GS-557, GS-559, GS-562, GS-564, GS-567, GS-568, GS-569, GS-570, GS-571, GS-572, GS-573, GS-574, GS-575, GS-576, GS-579, GS-580, GS-581, GS-583, GS-584, GS-585, GS-586, GS-587, GS-589, GS-592, GS-593, GS-594, GS-598, GS-602, GS-603, GS-604, GS-609, GS-610, GS-611, GS-612, GS-613, GS-614, GS-615, GS-616, GS-618, GS-620, GS-621, GS-622, GS-623, GS-625, GS-630, GS-633, GS-634, GS-635, or GS-637; or a pharmaceutically acceptable salt or solvate thereof.

54. The compound of any one of claims 21-51, wherein the compound is GS-060, GS-066, GS-105, GS-171, GS-173, GS-181, GS-182, GS-227, GS-243, GS-255, GS-261, GS-262, GS-264, GS-272, GS-286, GS-287, GS-305, GS-309, GS-314, GS-315, GS-339, GS-348, GS-418, GS-442, GS-444, GS-490, GS-510, GS-552, GS-568, GS-570, GS-579, GS-589, GS-613, or GS-614; or a pharmaceutically acceptable salt or solvate thereof.

55. The compound of any one of claims 21-51, wherein the compound is GS-171, GS-243, GS-255, GS-264, GS-286, GS-305, GS-442, GS-444, GS-568, GS-579, GS-589, GS-613, or GS-614; or a pharmaceutically acceptable salt or solvate thereof.

56. The compound of any of claims 21-55, wherein the compound inhibits growth of a cell with an IC₅₀ value below 8 nM.

57. The compound of any of claims 21-55, wherein the compound inhibits growth of a cell with an IC₅₀ value below 2 nM.

58. The compound of claim 56 or 57, wherein the cell comprises a MV4;11, or MOLM-13 cell.

59. A pharmaceutical composition comprising a compound of any one of claims 21-55, and a pharmaceutically acceptable excipient.
